(19) United States
(12) Patent Application Publication
(10) Pub. No.: US 2018/0071262 A1
(43) Pub. Date: Mar. 15, 2018

(54) SPIROCYCLIC HAT INHIBITORS AND METHODS FOR THEIR USE

(71) Applicant: AbbVie Inc., North Chicago, IL (US)

(72) Inventors: Edward A. Kesicki, Bothell, WA (US); Arthur F. Kluge, Lincoln, MA (US); Michael A. Patane, Andover, MA (US); John H. Van Drie, JR., North Andover, MA (US); Ce Wang, Beijing (CN); Maricel Torrent, Lake Bluff, IL (US); Michael L. Curtin, Pleasant Prairie, WI (US); Todd M. Hansen, Grayslake, IL (US); Rick F. Clark, Gurnee, IL (US); Robin R. Frey, Libertyville, IL (US); Zhiqin Ji, Libertyville, IL (US); Yujia Dai, Gurnee, IL (US); William J. McClellan, Waukegan, IL (US); Jane Gong, Deerfield, IL (US); Gui-dong G. Zhu, Gurnee, IL (US); Anthony Mastracchio, Vernon Hills, IL (US); Michael R. Michaelides, Libertyville, IL (US); Thomas D. Penning, Elmhurst, IL (US); Chunqiu C. Lai, Libertyvilee, IL (US)

(73) Assignee: AbbVie Inc., North Chicago, IL (US)

(21) Appl. No.: 15/727,220

(22) Filed: Oct. 6, 2017

Related U.S. Application Data
(63) Continuation of application No. PCT/CN2014/086792, filed on Sep. 18, 2014.
Application No. PCT/CN2014/086792 is a continuation of application No. 14/857,419, filed on Sep. 17, 2015.

(30) Foreign Application Priority Data
Sep. 18, 2014 (CN) PCT/CN2014/086792

(51) Int. Cl. A61K 31/4184; C07D 498/10; C07D 403/06; C07D 493/10; C07D 491/107; C07D 263/52; C07D 417/12; C07D 417/04; C07D 413/12; C07D 413/04; C07D 409/12; C07D 407/12; C07D 401/12; C07D 235/02; C07D 413/14; C07D 403/12; C07D 409/06; C07D 405/06; A61K 31/423; A61K 31/4439; C07D 401/06; C07D 495/10

(52) U.S. Cl. CPC A61K 31/4184; C07D 498/10; C07D 495/10; C07D 493/10; C07D 491/107; C07D 263/52; C07D 417/12; C07D 417/04; C07D 413/12; C07D 413/04; C07D 409/12; C07D 407/12; C07D 401/12; C07D 235/02; C07D 413/14; C07D 403/12; C07D 409/06; C07D 405/06; A61K 31/423; A61K 31/4439; C07D 401/06; C07D 403/06

(57) ABSTRACT

Compounds having a structure of Formula (IX):

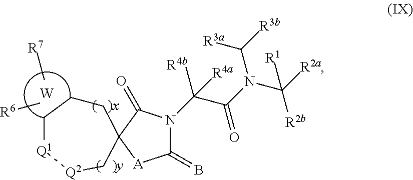

or a stereoisomer, tautomer or pharmaceutically acceptable salt thereof, wherein R¹, R^{2a}, R^{2b}, R^{3a}, R^{3b}, R^{4a}, R^{4b}, Q¹----Q², R⁶, R⁷, A, B, W, x, and y are as defined herein and are provided. Pharmaceutical compositions comprising such compounds and methods for treating various HAT-related conditions or diseases, including cancer, by administration of such compounds are also provided.

### RELATED APPLICATION INFORMATION

This application is a continuation of U.S. Non-provision patent application Ser. No. 14/857,419 filed on Sep. 17, 2015, is a continuation of PCT Patent Application CN2014/086792, filed on Sep. 18, 2014, and claims the benefit of U.S. Provisional Patent Application Ser. No. 62/157,253, filed on May 5, 2015, the contents of each of which are herein incorporated by reference.

### BACKGROUND

### Technical Field

The present invention relates to compounds that are inhibitors of HAT enzymes. The compounds find utility in a number of therapeutic applications, including the treatment of cancer.

### DESCRIPTION OF THE RELATED ART

Within the eukaryotic cell nucleus, DNA is condensed and packaged into chromatin. The structural unit of chromatin is a nucleosome, which consists of 147 base pairs of DNA wrapped 1.6 times around a histone core of two H2A-H2B dimers and a H3-H4 tetramer (Kornberg et al., 1999, Cell 98:285-294). Histones undergo extensive post-translational modification, which determines whether a gene is transcriptionally active or inactive (Goll and Bestor, 2002, Genes Dev. 16:1739-1742; Grant, 2001, Genome Biol. 2:). The reversible acetylation of histones and other proteins is one of the most abundant post-translational modifications in eukaryotic cells and is a major mechanism of cellular regulation. Histone acetyltransferases (HATs) catalyze the acetylation (transfer of an acetyl group) on a ϵ-amino group of a target lysine side chain within a substrate histone, and histone deacetylases (HDACs) catalyze the removal of acetyl groups from lysine residues. HATs are categorized into four major families based on primary sequence homology, shared structural features, and functional roles: Gcn5/PCAF (General control nonrepressed protein 5 and p300 and CBP associated factor); MYST (named for the founding members MOZ, Ybf2/Sas3, Sas2, and Tip60); p300/CBP (protein of 300 kDa and CREB Binding Protein); and Rtt109 (Regulator of Ty1 Transposition gene production 109).

Paralog HATs p300 (KATB) and CBP (referred to as p300/CBP) have >90% sequence identity and are conserved in metazoans. In addition to the enzymatic HAT domain, p300/CBP has multiple domains including three cysteine-histidine rich domains (CH1, CH2, and CH3), a KIX domain, a bromodomain, and a steroid receptor coactivator interaction domain (SRC-1 interaction domain). P300 and CBP were originally discovered as binding partners of E1A adenoviral protein and cAMP-regulated enhancer binding proteins, respectively (Yee and Branton, 1985, Virology 147:142-153; Harlow et al., 1986, Mol. Cell Biol. 6:1579-1589; Chrivia et al., 1993, Nature 365:855-859). P300/CBP was later found to have intrinsic HAT activity (Ogryzko et al., 1996, Cell 87:953-959; Bannister and Kouzarides, 1996, Nature 384:641-643). In addition to acetylating multiple lysines on all four core histones (H2A, H2B, H3 and H4), P300/CBP has been shown to have promiscuous acetyltransferase activity towards >70 substrates (Wang et al., 2008, Curr. Opin. Struct. Biol. 18:741-747), including, for example, p53 (Gu et al., 1997, Cell 90:595-606), MyoD (Polesskaya et al., 2002, J. Biol. Chem. 275:34359-64), STAT3 (Yuan et al., 2005, Science 307:269-73) and NFκβ (Chen et al., 2002, EMBO J. 21:6539-48). Besides acting as an acetyltransferase, p300 also acts as a scaffold for transcription factors or a bridge to connect the transcription factors and the basal transcriptional machinery to activate transcription (Chan and Thangue, 2001, J. Cell Sci. 114:2363-2373; Chen and Li, 2011, Epigenetics 6:957-961). P300/CBP proteins are involved in many cellular processes, including cell growth, proliferation, and differentiation (Chan and Thangue, supra).

Aberrant p300/CBP activity or mutations has been associated with various diseases. High p300 expression has been observed in prostate cancer (Heemers et al., 2008, Adv. Exp. Med. Biol. 617:535-40; Isharwal et al., 2008, Prostate 68:1097-104), liver cancer (Yokomizo et al., 2011, Cancer Lett. 310:1407; Li et al., 2011, J. Transl. Med. 9:5), and breast cancer (Fermento et al., 2010, Exp. Mol. Pathol. 88:256-64). Mutations in p300/CBP genes have been found in human tumors (Petrij et al., 1995, Nature 376:348-51; Muraoka et al, 1996, Oncogene 12:1565-69; Sobulo et al., 1997, Proc. Natl. Acad. Sci. USA 94:8732-37; Gayther et al., 2000, Nat. Genet. 24:300-304). P300 missense mutations and truncations have been found in solid tumors and B-cell lymphoma, suggesting a role as a tumor suppressor (Iyer et al., 2004, Oncogene 23:4225-31; Pasqualucci et al., 2011, nature 471:189-95). Inhibition of p300/CBP has therapeutic potential in cancer (Iyer et al., 2004, Proc. Natl. Acad. Sci. USA 101:7386-7391; Stimson et al., 2005, Mol. Cancer Ther. 4:1521-1532; Zheng et al., 2004, Methods Enzymol. 376:188-199), cardiac disease (Davidson et al., 2005, Chembiochem. 6:162-170); diabetes mellitus (Zhou et al., 2004, Nat. Med. 10:633-637), and HIV (Varier and Kundu, 2006, Curr. Pharm. Des. 12:1975-1993). Heterozygous germline mutations in CBP or p300 have also been described in Rubinstein-Taybi syndrome, an autosomal dominant disease characterized by mental retardation, skeletal abnormalities, and a high incidence of neoplasia (Petrij et al., 1995, Nature 376:348-51; Petrij et al., 2000, Am. J. Med. Genet. 92:47-52). P300/CBP is also involved in regulating inflammatory mediators (Deng et al., 2004, Blood 103:2135-42; Turner-Brannen et al., 2011, J. Immunol. 186:7127-7135). P300/CBP has also been linked to other diseases, such as fibrosis (Ghosh and Varga, 2007, J. Cell. Physiol. 213:663-671), metabolic syndrome (Bricambert et al., 2010, J. Clin. Invest. 120:4316-4331), and progressive neurodegenerative diseases, such as Huntington Disease (Cong et al., 2005, Mol. Cell. Neurosci. 30:12-23), Kennedy's disease (Lieberman et al., 2002, Hum. Mol. Genet. 11:1967-76), and Alzheimer's disease (Francis et al., 2007, Neurosci. Lett. 413:137-140).

The association of p300/CBP activity in disease pathogenesis suggests potential utility of p300/CBP as a therapeutic target. However, the identification of potent, specific histone acetyltransferase inhibitors has been challenging (Cole, 2008, Nat. Chem. Biol. 4:590-97). P300 HAT inhibitors derived from natural compounds have moderate potency but lack specificity (Dekker and Haisma, 2009, Drug Disc. Today 14:942-8). Lys-CoA, converted to a cell-permeable form with a Tat peptide attachment, is more selective, but has limited use in pharmacological studies due to its complexity. Recently, a selective p300 inhibitor C646 was identified using the Lys-CoA/p300 HAT structure in a virtual ligand screening approach (Bowers et al., 2010, Chemistry & Biology 17:471-482).

Accordingly, while progress has been made in this field, there remains a need in the art for improved HAT inhibitors. The present invention fulfills this need and provides further related advantages.

### BRIEF SUMMARY

In brief, the present invention is directed to compounds having activity as HAT inhibitors, including stereoisomers, tautomers, pharmaceutically acceptable salts and prodrugs thereof, and the use of such compounds to treat HAT-related conditions or diseases, such as cancer.

In one aspect, the present invention relates to compounds of Formula (IX),

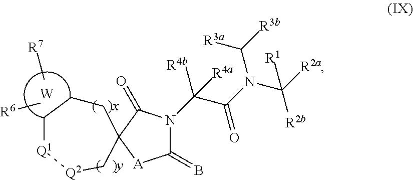

or a stereoisomer, tautomer or pharmaceutically acceptable salt thereof, wherein R¹, R^{2a}, R^{2b}, R^{3a}, R^{3b}, R^{4a}, R^{4b}, [symbol] , R⁶, R⁷, A, B, W, x, and y are as defined herein.

In one embodiment, of Formula (IX), A is —O—. In another embodiment of Formula (IX), A is —NR⁸—; and R⁸ is H. In another embodiment of Formula (IX), W is arylene. In another embodiment of Formula (IX), W is phenylene. In another embodiment of Formula (IX), W is phenylene, and R^{4a} and R^{4b} are each independently H.

In another aspect, the present invention relates to compounds of Formula (XIIa) or (XIIb),

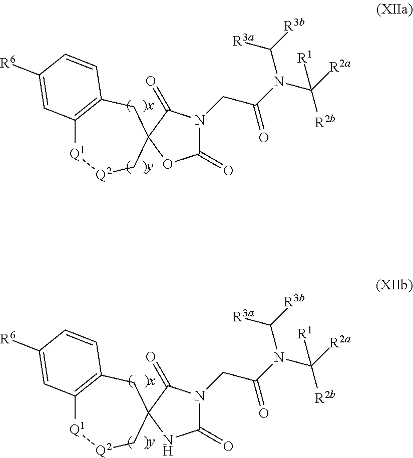

or a stereoisomer, tautomer or pharmaceutically acceptable salt thereof, wherein R¹, R^{2a}, R^{2b}, R^{3a}, R^{3b}, [symbol] , R⁶, x, and y are as defined herein.

In another aspect, the present invention relates to compounds of Formula (XIIIa) or (XIIIb),

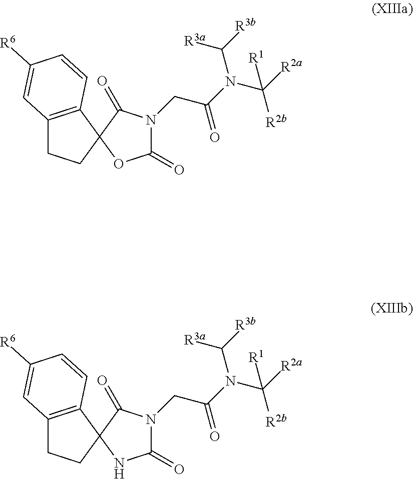

or a stereoisomer, tautomer or pharmaceutically acceptable salt thereof, wherein R¹, R^{2a}, R^{2b}, R^{3a}, R^{3b}, and R⁶ are as defined herein.

In one embodiment of Formula (XIIa) or (XIIb), R¹ is carbocyclyl. In one embodiment of Formula (XIIIa) or (XIIIb), R¹ is carbocyclyl. In another embodiment of Formula (XIIa) or (XIIb), R¹ is phenyl, which is unsubstituted. In another embodiment of Formula (XIIIa) or (XIIIb), R¹ is phenyl, which is unsubstituted. In another embodiment of Formula (XIIa) or (XIIb), R¹ is phenyl, which is substituted. In another embodiment of Formula (XIIIa) or (XIIIb), R¹ is phenyl, which is substituted. In another embodiment of Formula (XIIa) or (XIIb), R^{2a} and R^{2b} are each independently H. In another embodiment of Formula (XIIIa) or (XIIIb), R^{2a} and R^{2b} are each independently H. In another embodiment of Formula (XIIa) or (XIIb), R^{2a} is H; and R^{2b} is C₁-C₆ alkyl. In another embodiment of Formula (XIIIa) or (XIIIb), R^{2a} is H; and R^{2b} is C₁-C₆ alkyl. In another embodiment of Formula (XIIa) or (XIIb), R^{3a} is C₁-C₆ alkyl; and R^{3b} is CF₃. In another embodiment of Formula (XIIIa) or (XIIIb), R^{3a} is C₁-C₆ alkyl; and R^{3b} is CF₃. In another embodiment of Formula (XIIa) or (XIIb), R^{3a} is C₁-C₆ alkyl; and R^{3b} is cycloalkyl; wherein the R^{3a} C₁-C₆ alkyl is unsubstituted or substituted. In another embodiment of Formula (XIIIa) or (XIIIb), R^{3a} is C₁-C₆ alkyl; and R^{3b} is cycloalkyl; wherein the R^{3a} C₁-C₆ alkyl is unsubstituted or substituted. In another embodiment of Formula (XIIa) or (XIIb), R^{3a} is C₁-C₆ alkyl; and R^{3b} is cyclopropyl; wherein the R^{3a} C₁-C₆ alkyl is unsubstituted or substituted. In another embodiment of Formula (XIIIa) or (XIIIb), R^{3a} is C₁-C₆ alkyl; and R^{3b} is cyclopropyl; wherein the R^{3a} C₁-C₆ alkyl is unsubstituted or substituted. In another embodiment of Formula (XIIa) or (XIIb), R^{3a} is —CH₃; and R^{3b} is cyclopropyl. In another embodiment of Formula (XIIIa) or (XIIIb), R^{3a} is —CH₃; and R^{3b} is cyclopropyl.

Another embodiment pertains to a pharmaceutical composition comprising a pharmaceutically acceptable exicipient and a therapeutically effective amount of a compound of Formula (IX) or a pharmaceutically acceptable salt thereof.

Another embodiment pertains to a method of treating cancer in a patient, comprising administering to a patient suffering from a cancer a therapeutically effective amount of a compound of Formula (IX), or a pharmaceutically acceptable salt thereof.

Another embodiment pertains to a method of treating metabolic disease, neurogenerative disorders or inflammation in a patient, comprising administering to a patient suffering from metabolic disease, neurogenerative disorders or inflammation, a therapeutically effective amount of a compound of Formula (IX), or a pharmaceutically acceptable salt thereof.

Another embodiment pertains to a method of treating cancer in a patient, comprising administering to a patient suffering from a cancer a therapeutically effective amount of a compound of Formula (IX), or a pharmaceutically acceptable salt thereof, wherein said cancer is selected from the group consisting of acoustic neuroma, acute leukemia, acute lymphoblastic leukemia, acute myelogenous leukemia, acute t-cell leukemia, basal cell carcinoma, bile duct carcinoma, bladder cancer, brain cancer, breast cancer, bronchogenic carcinoma, Burkitt's lymphoma, cervical cancer, chondrosarcoma, chordoma, choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, dysplasias, metaplasias, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, gastric carcinoma, germ cell testicular cancer, gestational trophobalstic disease, glioblastoma, head and neck cancer, heavy chain disease, hemangioblastoma, hepatoma, hepatocellular cancer, hormone insensitive prostate cancer, leiomyosarcoma, liposarcoma, lung cancer, lymphangioendothelio-sarcoma, lymphangiosarcoma, lymphoblastic leukemia, lymphoma, malignancies and hyperproliferative disorders of the bladder, breast, colon, lung, ovaries, pancreas, prostate, skin and uterus, lymphoid malignancies of T-cell or B-cell origin, leukemia, medullary carcinoma, medulloblastoma, melanoma, meningioma, mesothelioma, multiple myeloma, myelogenous leukemia, myeloma, myxosarcoma, neuroblastoma, oligodendroglioma, oral cancer, osteogenic sarcoma, ovarian cancer, pancreatic cancer, papillary adenocarcinomas, papillary carcinoma, peripheral T-cell lymphoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors, stomach cancer, squamous cell carcinoma, synovioma, sweat gland carcinoma, testicular cancer, thyroid cancer, Waldenström's macroglobulinemia, testicular tumors, uterine cancer, and Wilms' tumor.

Another embodiment pertains to a method of treating cancer in a patient, comprising administering to a patient suffering from a cancer a therapeutically effective amount of a compound of Formula (IX), or a pharmaceutically acceptable salt thereof, further comprising administering a therapeutically effective amount of at least one additional therapeutic agent.

Another embodiment pertains to a method of treating metabolic disease, neurogenerative disorders or inflammation in a patient, comprising administering, to a patient suffering from metabolic disease, neurogenerative disorders or inflammation, a therapeutically effective amount of a compound of Formula (IX), or a pharmaceutically acceptable salt thereof, further comprising administering a therapeutically effective amount of at least one additional therapeutic agent.

These and other aspects of the invention will be apparent upon reference to the following detailed description. To this end, various references are set forth herein which describe in more detail certain background information, procedures, compounds and/or compositions, and are each hereby incorporated by reference in their entirety.

### BRIEF DESCRIPTION OF THE DRAWINGS

In the figures, identical reference numbers identify similar elements. The sizes and relative positions of elements in the figures are not necessarily drawn to scale and some of these elements are arbitrarily enlarged and positioned to improve figure legibility. Further, the particular shapes of the elements as drawn are not intended to convey any information regarding the actual shape of the particular elements, and have been solely selected for ease of recognition in the figures.

FIG. 1 illustrates inhibition of TNF-α induced IL-6 production in prostate cancer cells

FIG. 2 depicts inhibition of TNF-α induced IL-8 production in AML cells.

FIG. 3 shows inhibition of CoCl₂ induced VEGF production in DU145 cells.

FIG. 4 demonstrates inhibition of α-CD3/α-CD28 induced IFN-γ production in human PBMCs.

### DETAILED DESCRIPTION

In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment”, “another embodiment”, or “an embodiment” means that a particular feature, structure or characteristic described in connection with the embodiment is included in at least one embodiment of the present invention. Thus, the appearances of the phrases “in one embodiment”, “another embodiment”, or “in an embodiment” in various places throughout this specification are not necessarily all referring to the same embodiment. Furthermore, the particular features, structures, or characteristics may be combined in any suitable manner in one or more embodiments.

“Amino” refers to the —NH₂ radical.

“Aminocarbonyl” refers to the —C(═O)NH₂ radical.

“Aminosulfonyl” refers to the —S(O)₂NH₂ radical.

“Carboxyl” refers to the —CO₂H radical.

“Cyano” or “nitrilyl” refers to the —CN radical.

“Ester” refers to a structure of the formula RₐC(═O)R_{b} where Rₐ and R_{b} are each independently non-hydrogen substitutents (e.g., alkyl or aryl and the like).

“Hydrazone” refers to ═N—NH₂ substitutent.

“Hydroxy” or “hydroxyl” refers to the —OH radical.

“Imino” refers to the ═NH substituent.

“Nitro” refers to the —NO₂ radical.

“Oxo” refers to the ═O substituent.

“Oxime” refers to ═N—OH substitutent.

“Thioxo” refers to the ═S substituent.

“Ureayl” refers to the —NHC(═O)NH₂ radical.

“Alkyl” refers to a straight or branched hydrocarbon chain radical consisting solely of carbon and hydrogen atoms, which is saturated or unsaturated (i.e., contains one or more double (alkenyl) and/or triple (alkynyl) bonds), having from one to twelve carbon atoms (C₁-C₁₂ alkyl), from one to eight carbon atoms (C₁-C₈ alkyl) or from one to six carbon atoms (C₁-C₆ alkyl), and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, ethenyl, prop-1-enyl, but-1-enyl, pent-1-enyl, penta-1,4-dienyl, ethynyl, propynyl, butynyl, pentynyl, hexynyl, and the like. Unless stated otherwise specifically in the specification, an alkyl group may be optionally substituted.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double and/or triple bonds), and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Alkenyl” refers to an alkyl group which comprises one or more double bonds and has from one to twelve carbon atoms (C₁-C₁₂ alkenyl), from one to eight carbon atoms (C₁-C₈ alkenyl) or from one to six carbon atoms (C₁-C₆ alkenyl), and which is attached to the rest of the molecule by a single bond, e.g., ethenyl, propenyl, butenyl, pentenyl, hexenyl, and the like. Unless stated otherwise specifically in the specification, an alkenyl group may be optionally substituted.

“Alkynyl” refers to an alkyl group which comprises one or more triple bonds and has from one to twelve carbon atoms (C₂-C₁₂ alkynyl), from one to eight carbon atoms (C₂-C₈ alkynyl) or from one to six carbon atoms (C₂-C₆ alkynyl), and which is attached to the rest of the molecule by a single bond, e.g., ethynyl, propynyl, butynyl, pentynyl, hexynyl, and the like. Unless stated otherwise specifically in the specification, an alkynyl group may be optionally substituted.

“Alkoxy” refers to a radical of the formula —OR^{a} where R^{a} is an alkyl radical as defined above. Unless stated otherwise specifically in the specification, an alkoxy group may be optionally substituted.

“Alkoxyalkyl” refers to a radical of the formula —R^{a}OR_{b} where R^{a} is alkylene radical as defined above and R_{b} is an alkyl radical as defined above. Unless stated otherwise specifically in the specification, an alkoxyalkyl group may be optionally substituted.

“Alkoxycarbonyl” refers to a radical of the formula —C(═O)OR^{a} where R^{a} is an alkyl radical as defined above. Unless stated otherwise specifically in the specification, an alkoxycarbonyl group may be optionally substituted.

“Alkylaminyl” or “alkylamino” refers to a radical of the formula —NHRₐ or —NRₐR^{a} where each Rₐ is, independently, an alkyl radical as defined above containing one to twelve carbon atoms. Unless stated otherwise specifically in the specification, an alkylaminyl group may be optionally substituted.

“Aminocarbonylalkyl” refers to a radical of the formula —R^{a}(═O)NH₂ or where R^{a} is an alkylene radical as defined above. Unless stated otherwise specifically in the specification, an aminocarbonylalkyl group may be optionally substituted.

“Aryl” refers to a hydrocarbon ring system radical comprising 6 to 18 carbon atoms and at least one aromatic ring. For purposes of this invention, the aryl radical may be a monocyclic, bicyclic, tricyclic or tetracyclic ring system, which may include spiro, fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Arene” refers to a hydrocarbon ring system comprising 6 to 18 carbon atoms and at least one aromatic ring. For purposes of this invention, the arene may be a monocyclic, bicyclic, tricyclic or tetracyclic ring system, which may include spiro, fused or bridged ring systems. Arenes include, but are not limited to, aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “arene” is meant to include arenes that are optionally substituted.

“Arylene” refers to a bivalent hydrocarbon ring system radical comprising 6 to 18 carbon atoms and at least one aromatic ring. For purposes of this invention, the arylene may be a monocyclic, bicyclic, tricyclic or tetracyclic ring system, which may include spiro, fused or bridged ring systems. Arylenes include, but are not limited to, those derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “arylene” is meant to include arenes that are optionally substituted.

“Aralkyl” refers to a radical of the formula —R_{b}—R_{c} where R_{b} is an alkylene chain as defined above and R_{c} is one or more aryl radicals as defined above. Examples of aralkyl include benzyl, diphenylmethyl and the like. Unless stated otherwise specifically in the specification, an aralkyl group may be optionally substituted.

“Aryloxy” refers to a radical of the formula —ORₐ where Rₐ is an aryl radical as defined above, for example phenoxy. Unless stated otherwise specifically in the specification, an aryloxy group may be optionally substituted.

“Carbocyclyl” or “carbocyclic ring” refers to a monocyclic or polycyclic hydrocarbon radical consisting solely of carbon ring atoms, which may include spiro, fused or bridged ring systems, having from three to fifteen carbon atoms, preferably having from three to ten carbon atoms, and which is saturated or unsaturated and attached to the rest of the molecule by a single bond. Carbocycles include cycloalkyls and aryls as defined herein. Unless stated otherwise specifically in the specification, a carbocyclyl group may be optionally substituted.

“Cycloalkyl” refers to a stable non-aromatic monocyclic or polycyclic hydrocarbon radical consisting solely of carbon ring atoms, which may include spiro, fused or bridged ring systems, having from three to fifteen carbon atoms, preferably having from three to ten carbon atoms, and which is saturated or partially unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group may be optionally substituted.

“Cycloalkane” refers to a stable non-aromatic monocyclic or polycyclic hydrocarbon consisting solely of carbon ring atoms, which may include spiro, fused or bridged ring systems, having from three to fifteen carbon atoms, preferably having from three to ten carbon atoms, and which is saturated or partially unsaturated and attached to the rest of the molecule by a single bond. Monocyclic rings include, for example, cyclopropane, cyclobutane, cyclopentane, cyclohexane, cycloheptane, and cyclooctane. Polycyclic radicals include, for example, adamantane, norbornane, decalin, 7,7-dimethyl-bicyclo[2.2.1]heptane, and the like. Unless otherwise stated specifically in the specification, a cycloalkane group may be optionally substituted.

“Cycloalkylalkyl” refers to a radical of the formula —R_{b}R_{d} where R_{b} is an alkylene chain as defined above and Rd is a cycloalkyl radical as defined above. In certain embodiments, R_{b} is substituted with a further cycloalkyl group, such that the cycloalkylalkly comprises two cycloalkyl moieties. Cyclopropylalkyl and cyclobutylalkyl are exemplary cycloalkylalkyl groups, comprising at least one cyclopropyl or at least one cyclobutyl group, respectively. Unless stated otherwise specifically in the specification, a cycloalkylalkyl group may be optionally substituted.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” refers to an alkoxy radical, as defined above, that is substituted by one or more halo radicals, as defined above, e.g., trifluoromethoxy, difluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

“Haloalkoxyalkyl” refers to a radical of the formula —RₐOR_{b} where Rₐ is an alkylene radical as defined above containing one to twelve carbon atoms and R_{b} is a haloalkyl radical as defined above. Unless stated otherwise specifically in the specification, a haloalkoxyalkyl group may be optionally substituted.

“Heterocyclyl” or “heterocyclic ring” refers to a stable 3- to 18-membered non-aromatic or aromatic ring radical which consists of two to twelve carbon and from one to six heteroatom ring atoms selected from the group consisting of nitrogen, oxygen and sulfur. Heterocyclyl includes heteroaryls as defined herein. Unless stated otherwise specifically in the specification, the heterocyclyl radical may be a monocyclic, bicyclic, tricyclic or tetracyclic ring system, which may include spiro, fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isozazolidinyl, morpholinyl, octahydroindolyl, octahydroisoindolyl, 2-oxopiperazinyl, 2-oxopiperidinyl, 2-oxopyrrolidinyl, ozazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

“Heterocycle” refers to a stable 3- to 18-membered non-aromatic or aromatic ring which consists of two to twelve carbon and from one to six heteroatom ring atoms selected from the group consisting of nitrogen, oxygen and sulfur. Heterocycle includes heteroarenes as defined herein. Unless stated otherwise specifically in the specification, the heterocycle may be a monocyclic, bicyclic, tricyclic or tetracyclic ring system, which may include spiro, fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocycles include, but are not limited to, dioxolane, thienyl[1,3]dithiane, decahydroisoquinoline, imidazoline, imidazolidine, isothiazolidine, isozazolidine, morpholine, octahydroindole, octahydroisoindole, 2-oxopiperazine, 2-oxopiperidine, 2-oxopyrrolidine, ozazolidine, piperidine, piperazine, 4-piperidone, pyrrolidine, pyrazolidine, quinuclidine, thiazolidine, tetrahydrofuran, trithiane, tetrahydropyran, thiomorpholine, thiamorpholine, 1-oxo-thiomorpholine, and 1,1-dioxo-thiomorpholine. Unless stated otherwise specifically in the specification, a heterocycle group may be optionally substituted.

“Hydroxylheterocyclyl” is a heterocyclyl radical as defined above substituted with one or more hydroxyl radicals. Unless stated otherwise specifically in the specification, a hydroxylheterocyclyl group may be optionally substituted.

“Heterocyclylalkyl” refers to a radical of the formula —R_{b}Rₑ where R_{b} is an alkylene chain as defined above and Rₑ is a heterocyclyl radical as defined above, and if the heterocyclyl is a nitrogen-containing heterocyclyl, the heterocyclyl may be attached to the alkyl radical at the nitrogen atom. Unless stated otherwise specifically in the specification, a heterocyclylalkyl group may be optionally substituted.

“Heterocyclyloxy” refers to a radical of the formula —OR^{a} where R^{a} is heterocyclyl radical as defined above. Unless stated otherwise specifically in the specification, an heterocyclyloxy group may be optionally substituted.

“Heteroaryl” refers to a 5- to 14-membered ring system radical comprising one to thirteen carbon and one to six heteroatom ring atoms selected from the group consisting of nitrogen, oxygen and sulfur, and at least one aromatic ring. For purposes of this invention, the heteroaryl radical may be a monocyclic, bicyclic, tricyclic or tetracyclic ring system, which may include spiro, fused or bridged ring systems; and the nitrogen, carbon or sulfur atoms in the heteroaryl radical may be optionally oxidized; the nitrogen atom may be optionally quaternized. The heteroatom may be a member of an aromatic or non-aromatic ring, provided at least one ring in the heteroaryl is aromatic. Examples include, but are not limited to, azepinyl, acridinyl, benzimidazolyl, benzothiazolyl, benzindolyl, benzodioxolyl, benzofuranyl, benzooxazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

“Heteroarene” refers to a 5- to 14-membered ring system comprising one to thirteen carbon and one to six heteroatom ring atoms selected from the group consisting of nitrogen, oxygen and sulfur, and at least one aromatic ring. For purposes of this invention, the heteroarene may be a monocyclic, bicyclic, tricyclic or tetracyclic ring system, which may include spiro, fused or bridged ring systems; and the nitrogen, carbon or sulfur atoms in the heteroaryl radical may be optionally oxidized; the nitrogen atom may be optionally quaternized. The heteroatom may be a member of an aromatic or non-aromatic ring, provided at least one ring in the heteroaryl is aromatic. Examples include, but are not limited to, azepine, acridine, benzimidazole, benzothiazole, benzindole, benzodioxole, benzofuran, benzooxazole, benzothiadiazole, benzo[b][1,4]dioxepine, 1,4-benzodioxane, benzonaphthofuran, benzoxazole, benzodioxole, benzodioxine, benzopyran, benzopyranone, benzofuran, benzofuranone, benzothiene (benzothiophene), benzotriazole, benzo[4,6]imidazo[1,2-a]pyridine, carbazole, cinnoline, dibenzofuran, dibenzothiophene, furan, furanone, isothiazole, imidazole, indazole, indole, indazole, isoindole, indoline, isoindoline, isoquinoline, indolizine, isoxazole, naphthyridine, oxadiazole, 2-oxoazepine, oxazole, oxirane, 1-oxidopyridine, 1-oxidopyrimidine, 1-oxidopyrazine, 1-oxidopyridazine, 1-phenyl-1H-pyrrole, phenazine, phenothiazine, phenoxazine, phthalazine, pteridine, purine, pyrrole, pyrazole, pyridine, pyrazine, pyrimidine, pyridazine, quinazoline, quinoxaline, quinoline, quinuclidine, isoquinoline, tetrahydroquinoline, thiazole, thiadiazole, triazole, tetrazole, triazine, and thiophene (i.e. thiene). Unless stated otherwise specifically in the specification, a heteroarene group may be optionally substituted.

“Heteroarylene” refers to a bivalent 5- to 14-membered ring system radical comprising one to thirteen carbon and one to six heteroatom ring atoms selected from the group consisting of nitrogen, oxygen and sulfur, and at least one aromatic ring. For purposes of this invention, the heteroarylene may be a monocyclic, bicyclic, tricyclic or tetracyclic ring system, which may include spiro, fused or bridged ring systems; and the nitrogen, carbon or sulfur atoms in the heteroaryl radical may be optionally oxidized; the nitrogen atom may be optionally quaternized. The heteroatom may be a member of an aromatic or non-aromatic ring, provided at least one ring in the heteroaryl is aromatic. Examples include, but are not limited to, those derived from azepine, acridine, benzimidazole, benzothiazole, benzindole, benzodioxole, benzofuran, benzooxazole, benzothiadiazole, benzo[b][1,4]dioxepine, 1,4-benzodioxane, benzonaphthofuran, benzoxazole, benzodioxole, benzodioxine, benzopyran, benzopyranone, benzofuran, benzofuranone, benzothiene (benzothiophene), benzotriazole, benzo[4,6]imidazo[1,2-a]pyridine, carbazole, cinnoline, dibenzofuran, dibenzothiophene, furan, furanone, isothiazole, imidazole, indazole, indole, indazole, isoindole, indoline, isoindoline, isoquinoline, indolizine, isoxazole, naphthyridine, oxadiazole, 2-oxoazepine, oxazole, oxirane, 1-oxidopyridine, 1-oxidopyrimidine, 1-oxidopyrazine, 1-oxidopyridazine, 1-phenyl-1H-pyrrole, phenazine, phenothiazine, phenoxazine, phthalazine, pteridine, purine, pyrrole, pyrazole, pyridine, pyrazine, pyrimidine, pyridazine, quinazoline, quinoxaline, quinoline, quinuclidine, isoquinoline, tetrahydroquinoline, thiazole, thiadiazole, triazole, tetrazole, triazine, and thiophene (i.e. thiene). Unless stated otherwise specifically in the specification, a heteroarylene group may be optionally substituted.

“Heteroarylalkyl” refers to a radical of the formula —R_{b}R_{f} where R_{b} is an alkylene chain as defined above and R_{f} is a heteroaryl radical as defined above. Unless stated otherwise specifically in the specification, a heteroarylalkyl group may be optionally substituted.

“Hydroxylalkyl” is an alkyl radical as defined above substituted with one or more hydroxyl radicals. Unless stated otherwise specifically in the specification, a hydroxylalkyl group may be optionally substituted.

“Hydroxylalkynyl” is an alkynyl radical as defined above substituted with one or more hydroxyl radicals. Unless stated otherwise specifically in the specification, a hydroxylalkynyl group may be optionally substituted.

“Nitrilylalkyl” is an alkyl radical as defined above substituted with one or more nitrilyl radicals. Unless stated otherwise specifically in the specification, a nitrilyalkyl group may be optionally substituted.

“Thioalkyl” refers to a radical of the formula —SRₐ where Rₐ is an alkyl radical as defined above containing one to twelve carbon atoms. Unless stated otherwise specifically in the specification, a thioalkyl group may be optionally substituted.

The term “substituted” used herein means any of the above groups (i.e., alkyl, alkylene, alkenyl, alkynyls, alkoxy, alkoxyalkyl, alkoxycarbonyl, alkylaminyl, aminocarbonylalkyl, aryl, aralkyl, aryloxy, carbocyclyl, cycloalkyl, cycloalkylalkyl, haloalkyl, haloalkoxy, haloalkoxyalkyl, heterocyclyl, heterocyclylalkyl, heteroaryl, heteroarylalkyl, hydroxylalkyl, hydroxylalkynyl, nitrilylalkyl and/or thioalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, —NSO₂R_{g}, —NR_{g}SO₂NR_{g}C(═O)Rₕ, —SO₂NR_{g}C(═O)ORₕ and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, heterocyclylalkyl, heteroaryl, and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, hydroxylheterocyclyl, heterocyclylalkyl, heteroaryl, and/or heteroarylalkyl group. In addition, each of the foregoing substituents may also be optionally substituted with one or more of the above substituents.

“Prodrug” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987.

The term “prodrug” is also meant to include any covalently bonded carriers, which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the invention and the like.

Certain embodiments of the invention disclosed herein are meant to encompass all pharmaceutically acceptable compounds of structure (I) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds could be useful to help determine or measure the effectiveness of the compounds, by characterizing, for example, the site or mode of action, or binding affinity to pharmacologically important site of action. Certain isotopically-labelled compounds of structure (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of structure (I) can generally be prepared by conventional techniques known to those skilled in the art or by processes analogous to those described in the Preparations and Examples as set out below using an appropriate isotopically-labeled reagent in place of the non-labeled reagent previously employed.

Embodiments of the invention disclosed herein are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the invention includes compounds produced by a process comprising administering a compound of this invention to a mammal for a period of time sufficient to yield a metabolic product thereof. Such products are typically identified by administering a radiolabelled compound of the invention in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets (e.g., cats, dogs, swine, cattle, sheep, goats, horses, rabbits), and non-domestic animals such as wildlife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Effective amount” or “therapeutically effective amount” refers to that amount of a compound of the invention which, when administered to a mammal, preferably a human, is sufficient to effect treatment, as defined below, of a HAT related condition or disease in the mammal, preferably a human. The amount of a compound of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the invention, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

The chemical naming protocol and structure diagrams used herein are a modified form of the I.U.P.A.C. nomenclature system, using the ACD/Name Version 2012 (Build 56084, 5 Apr. 2012 or Build 59026, 3 Sep. 2012) software program and/or ChemDraw Ultra Version 9.0.7, 11.0.1, or 12.0 software naming program (CambridgeSoft). For complex chemical names employed herein, a substituent group is generally named before the group to which it attaches. For example, cyclopropylethyl comprises an ethyl backbone with cyclopropyl substituent. Unless specifically stated otherwise, all bonds are identified in the chemical structure diagrams herein, except for some carbon atoms, which are assumed to be bonded to sufficient hydrogen atoms to complete the valency.

### I. Compounds

In one embodiment, the present disclosure provides a compound having the following structure (I):

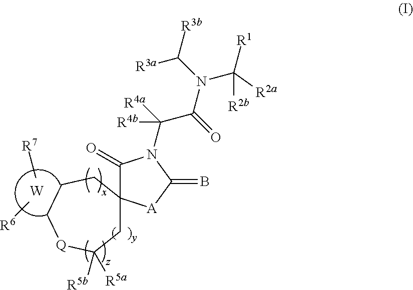

or a stereoisomer, tautomer or pharmaceutically acceptable salt thereof, wherein:
A is —NR⁸—, —O—, or —S—;
B is O or NH;
Q is —CHR¹⁰, —O—, —S(O)ₘ—, —NR⁹— or absent;
W is an aryl or heteroaryl ring;
R¹ is carbocyclyl or heterocyclyl;
R^{2a} and R^{2b} are each independently H, D, or C₁-C₆ alkyl;
R^{3a} is cycloalkyl or heterocyclyl and R^{3b} is C₁-C₆ alkyl or cycloalkyl; or R^{3a} and R^{3b} are each independently C₂-C₆ alkyl; or R^{3a} and R^{3b} taken together form a heterocycle; or R^{3a} is C₁-C₆ alkyl and R^{3b} is C₁-C₆ substituted alkyl;
R^{4a} and R^{4b} are each independently H, D or C₁-C₆alkyl;
R^{5a} and R^{5b} are each independently H, D, —OH, —CO₂H, —CN, —CO₂NH₂, —C₁-C₆ alkyl or alkoxy; or R^{5a} and R^{5b} taken together form oxo or ═N—OR¹¹;
R⁶ and R⁷ are each independently H, halo, —OH, —CN, —CO₂H, C₁-C₆ alkyl, C₁-C₆ alkynyl, alkoxy, haloalkoxy, alkoxyalkyl, haloalkoxyalkyl, hydroxylalkyl, hydroxylalkynyl, cycloalkyl, heterocyclyl, heterocyclylalkyl, heterocyclyloxy, —B(R¹¹)(R¹³), —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹² or —NHS(O)ₘR¹²;
R⁸ and R⁹ are each independently H or C₁-C₆alkyl;
R¹⁰ is H, OH, —CN, —CO₂R¹², —C(═O)NHR¹³, C₁-C₆ alkyl or alkoxy;
R¹¹ and R¹³ are each independently H, OH or C₁-C₆alkyl;
R¹² is, at each occurrence, independently H, C₁-C₆ alkyl, cycloalkyl, heterocyclyl or heterocyclyalkyl;
m is, at each occurrence, independently 0, 1 or 2; and
x, y and z are each independently 0 or 1, wherein x, y and z are selected such that the sum of x+y+z is 1 or 2;

with the proviso that R^{3a} and R^{3b} are not cyclopropyl and methyl, respectively, when R¹ and W are each unsubstituted phenyl and Q is —CH₂—.

Some embodiments of the invention also include deuterium substituted compounds of structure (I). The deuterium may be included at various positions in the compound, for example in some embodiments one or more of R^{2a}, R^{2b}, R^{4a} and/or R^{4b} are deuterium. In other embodiments, substituents, such as R¹, are substituted with one or more deuterium atoms. While not wishing to be bound by theory, such deuterium substitutions may contribute to advantageous metabolism of the compounds.

In certain embodiments, W is an aryl ring, such as a phenyl ring. For example, in some embodiments, the compound has the following structure (II):

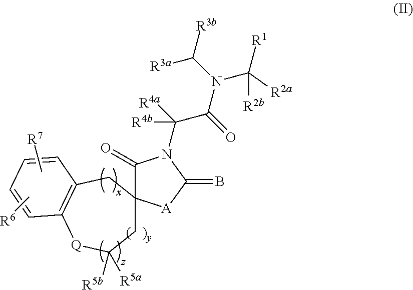

In certain other embodiments, the compound has one of the following structures (IIa), (IIb), (IIc), (IId), (IIe), (IIf), (IIg) or (IIh):

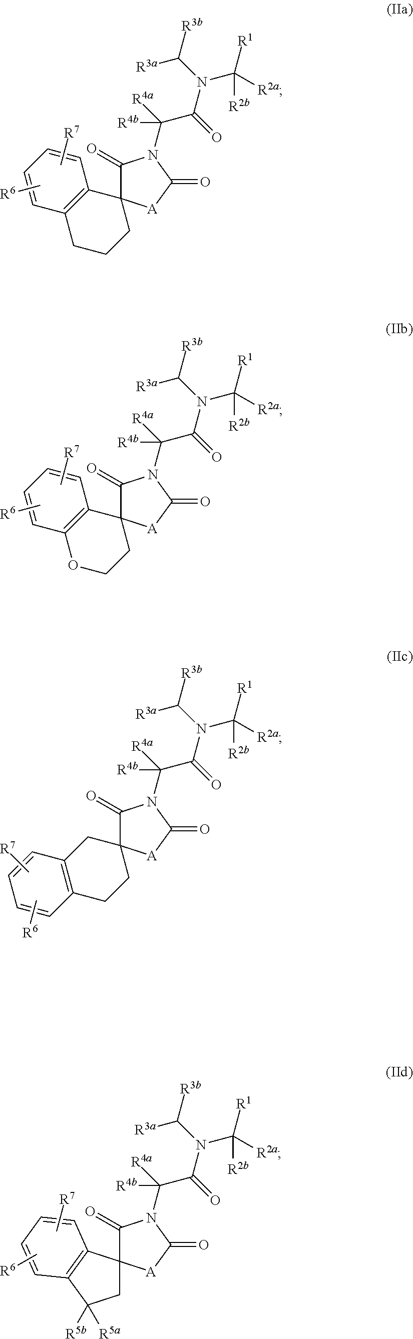

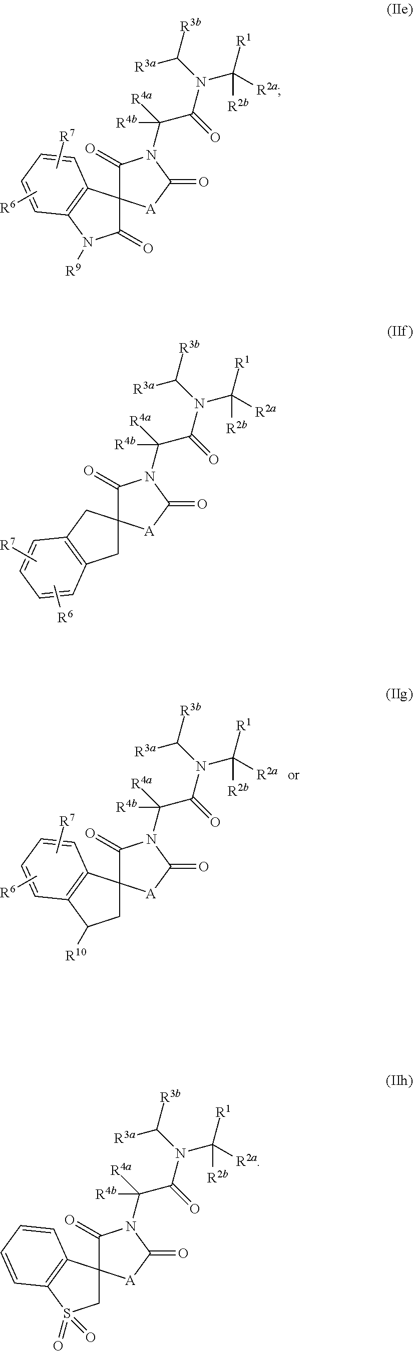

In various other embodiments, W is a heteroaryl ring. For example, in some embodiments, the heteroaryl ring is a 5 or 6-membered heteroaryl ring. In other embodiments, the heteroaryl ring comprises nitrogen or sulfur. In some exemplary embodiments, the compound has one of the following structures (IIIa), (IIIb), (IIIc) or (IIId):

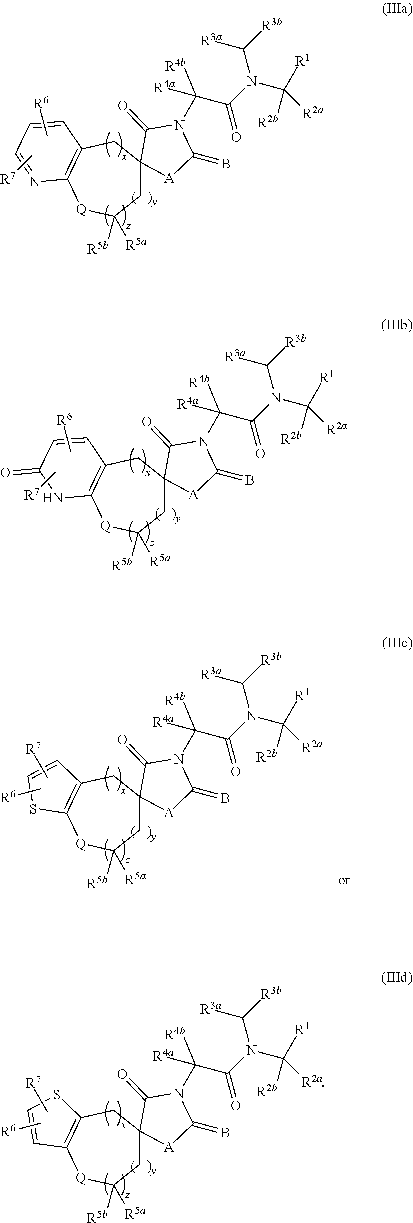

In any of the foregoing embodiments, R¹ is unsubstituted. In other of any of the foregoing embodiments, R¹ is substituted with one or more substitutents. In certain embodiments, R¹ is substituted with one or more deuterium atoms. For example, in some of the foregoing embodiments R¹ is substituted with one or more of D, OH, alkyl, amino, alkylamino, alkynyl, alkoxy, halo, —S(O)ₘalkyl, nitrilylalkyl, nitrilyl, ureayl, aminocarbonyl or aminocarbonylalkyl, wherein m is 0, 1 or 2. In some other embodiments of any of the foregoing, R¹ is substituted with one or more of fluoro, chloro, bromo, methyl, ethynyl, methylsulfanyl, methylsulfoxide, methoxy, acetonitrilyl, ureayl, aminocarbonyl or acetamidyl.

In other of any of the foregoing embodiments, R¹ is aryl. For example, in some embodiments R¹ is napthyl. In some of these embodiments, R¹ has the following structure:

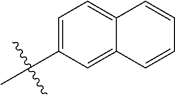 [SMILES: *c1ccc2ccccc2c1]

In some embodiments, one or more of the hydrogen atoms in the naphthyl ring are substituted for deuterium atoms.

In some other of the foregoing embodiments, R¹ is phenyl. For example, in some embodiments R¹ has one of the following structures:

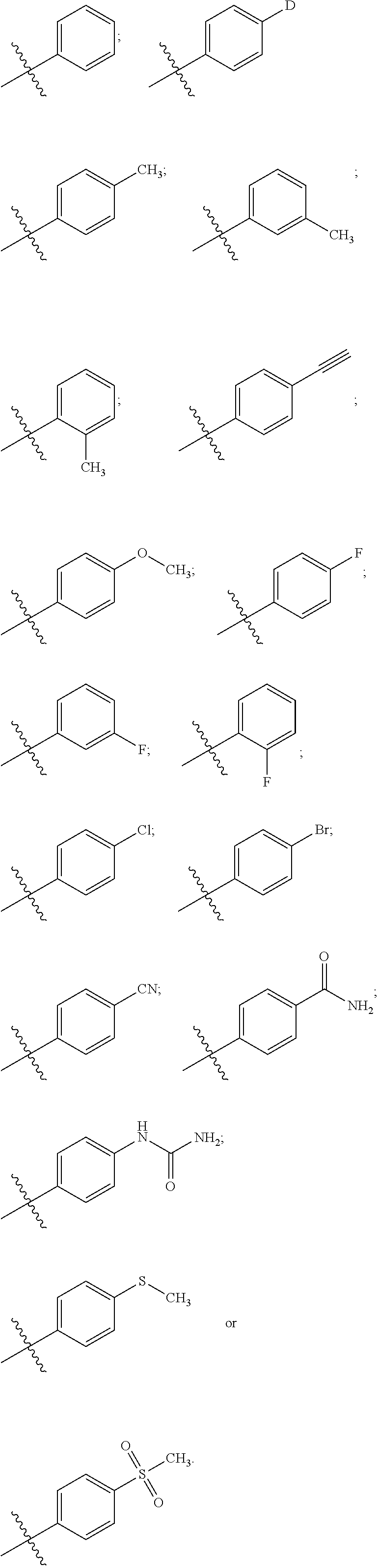

In some embodiments of the foregoing, one or more of the hydrogen atoms in any of the phenyl rings are substituted for deuterium atoms.

In still other of the foregoing embodiments, R¹ is heteroaryl. For example, in some embodiments, R¹ is triazolyl, furanyl, thiophenyl, pyridinyl or indolyl. In certain embodiments, R¹ has one of the following structures:

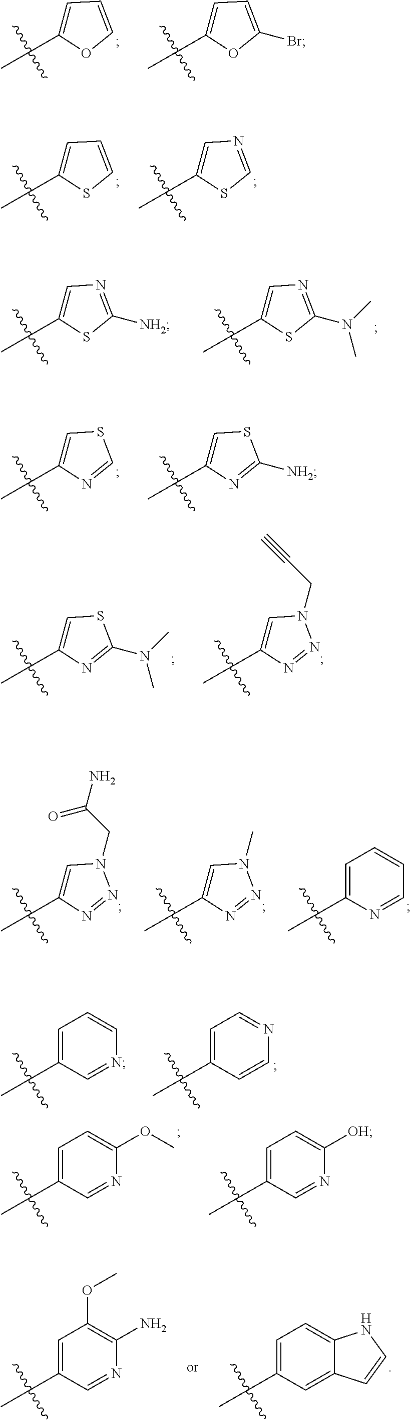

In some embodiments, one or more of the hydrogen atoms in the R¹ heteroaryl ring are substituted for deuterium atoms.

In any of the foregoing embodiments, one of R^{2a} or R^{2b} is H. In other of the foregoing embodiments, each of R^{2a} and R^{2b} is H.

In still other of the foregoing embodiments, one of R^{2a} or R^{2b} is C₁-C₆ alkyl. For example, in some embodiments, the C₁-C₆ alkyl is methyl.

In yet other of the foregoing embodiments, R^{3a} is cycloalkyl. For example, in some embodiments cycloalkyl is cyclopropyl or cyclobutyl. In some embodiments cycloalkyl is cyclopropyl. In some other embodiments cycloalkyl is cyclobutyl.

In more embodiments of the foregoing, R^{3a} is heterocyclyl. In some of these embodiments, heterocyclyl is oxetanyl. In some other of these embodiments, heterocyclyl is azetinyl.

In yet other embodiments of the foregoing, R^{3b} is C₁-C₆ alkyl, for example in some embodiments R^{3b} is methyl. In some other embodiments, R^{3b} is haloalkyl, for example trifluoromethyl.

In more embodiments of the foregoing, R^{3b} is cycloalkyl, such as cyclopropyl.

In some of the foregoing embodiments, R^{3a} and R^{3b} are each independently C₂-C₆ alkyl. For example, in some embodiments R^{3a} and R^{3b} are each ethyl.

In some embodiments, R^{3a} and R^{3b} taken together form a heterocycle. For example, in certain embodiments heterocyclyl is tetrahydropyranyl or tetrahydrofuranyl.

In other different embodiments, R^{3a} is C₁-C₆ alkyl and R^{3b} is C₁-C₆ substituted alkyl. In some of these embodiments C₁-C₆ substituted alkyl is haloalkyl, such as trifluoromethyl. In other embodiments, R^{3b} is alkoxy-substituted alkyl. In more of the foregoing embodiments, R^{3a} is methyl.

In still other embodiments of any of the foregoing, R^{4a} and R^{4b} are each H.

In some embodiments of any of the foregoing embodiments, R^{5a} and R^{5b}, when present, are each H or are each methyl. In other embodiments, one of R^{5a} or R^{5b}, when present, is D, —OH, —CO₂H, —CN, —CO₂NH₂ or alkoxy. In other embodiments, R^{5a} and R^{5b}, when present, join to form oxo. For example, in some embodiments R^{5a} and R^{5b}, when present, join to form ═NOR¹¹. In some embodiments, R¹¹ is H or methyl.

In still more embodiments of any of the foregoing, one of R⁶ or R⁷ is H.

In some embodiments of any of the foregoing, R⁷ is H, and the compound has one of the following structures (IIh), (IIi), (IIj) or (IIk):

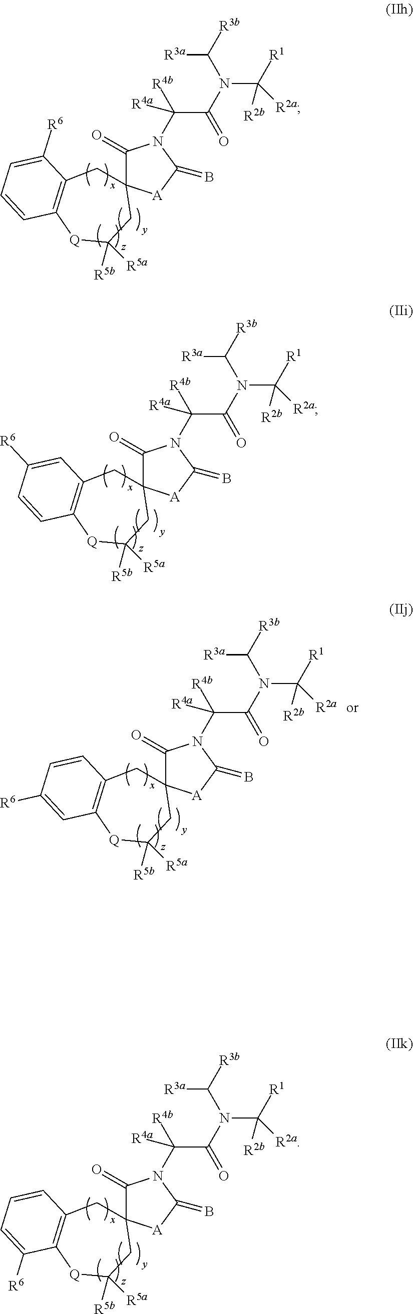

In more of any of the foregoing embodiments, R⁶ is halo, for example, F, Cl or Br.

In other embodiments of the foregoing embodiments, R⁶ is —OH.

In more embodiments of the foregoing embodiments, R⁶ is —CN.

In yet more embodiments of the foregoing embodiments, R⁶ is —CO₂H.

In still more embodiments of the foregoing embodiments, R⁶ is C₁-C₆ alkyl, such as methyl.

In other embodiments of the foregoing embodiments, R⁶ is C₁-C₆ alkynyl, for example ethynyl.

In other examples of the foregoing embodiments, R⁶ is alkoxy, such as methoxy.

In different embodiments of any of the compounds of structure (I) R⁶ is —NHC(═O)R¹². For example, in some embodiments R⁶ has one of the following structures:

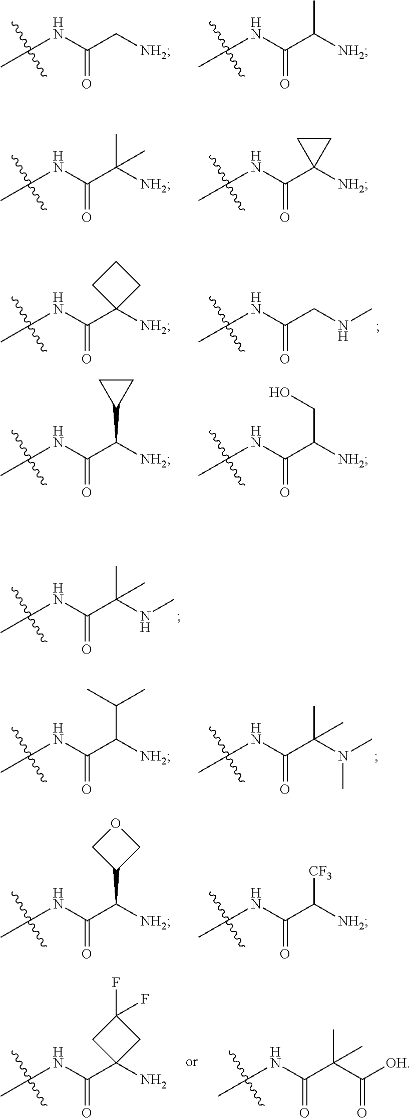

In yet more embodiments, R⁶ is haloalkoxy, for example, in some embodiments R⁶ is difluoromethoxy.

In other embodiments of the foregoing embodiments, R⁶ is alkoxyalkyl.

In still other embodiments of the foregoing embodiments, R⁶ is haloalkoxyalkyl.

In yet more embodiments of the foregoing embodiments, R⁶ is hydroxylalkyl.

In other embodiments of the foregoing embodiments, R⁶ is hydroxylalkynyl.

In more examples of the foregoing embodiments, R⁶ is cycloalkyl, for example in some embodiments R⁶ is cyclopropyl.

In other embodiments of the foregoing embodiments, R⁶ is heterocyclyl. For example, in some embodiments R⁶ is azetidinyl, pyrazolyl, imidazolyl, piperidinyl, piperizinyl, tetrahydrofuranyl, morpholinyl, furanyl, pyridinyl, triazolyl, tetrazolyl, pyrrolidinyl, thiazolyl, pyrimidindionyl or pyridinonyl.

In still other embodiments of the foregoing embodiments, R⁶ is heterocyclylalkyl, such as piperadinylalkyl, piperizinylalkyl, morpholinylalkyl, azetidinylalkyl or oxetanylalkyl.

In even more embodiments, R⁶ is —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹² or —NHS(O)ₘR¹². In certain embodiments, R¹² is, at each occurrence, H. In other embodiments, at least one R¹² is C₁-C₆ alkyl. In still other embodiments at least one R¹² is cycloalkyl. In other embodiments, at least one R¹² is heterocyclyl.

In further embodiments of the immediately foregoing embodiments, R¹² is unsubstituted. In other embodiments, R¹² is substituted. For example, in some embodiments R¹² is substituted with one or more of amino, nitrilyl, hydroxyl, alkoxy, oxo, carboxyl, alkoxycarbonyl, heterocyclyl or alkenyl.

In certain embodiments of any of the foregoing embodiments, R⁶ has one of the following structures:

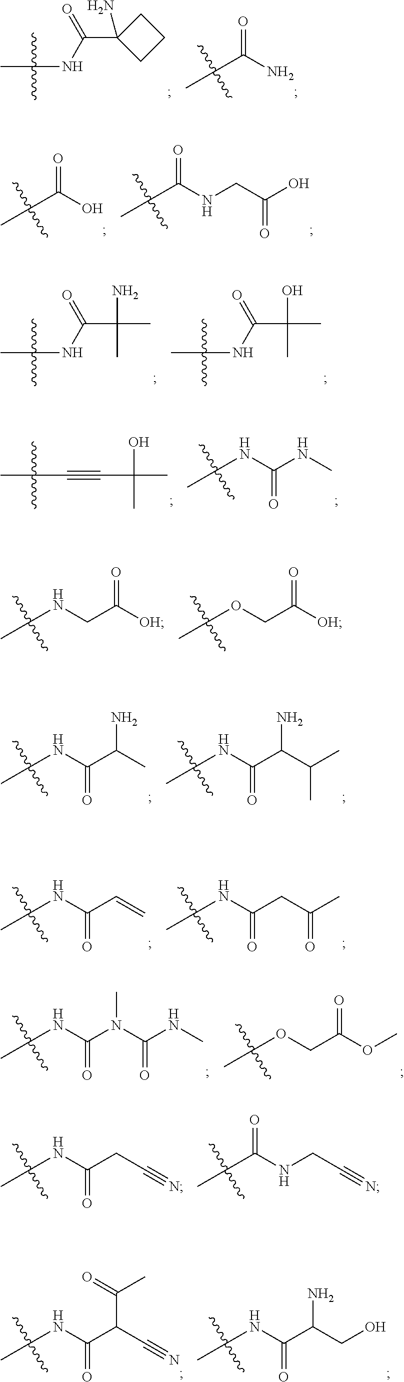

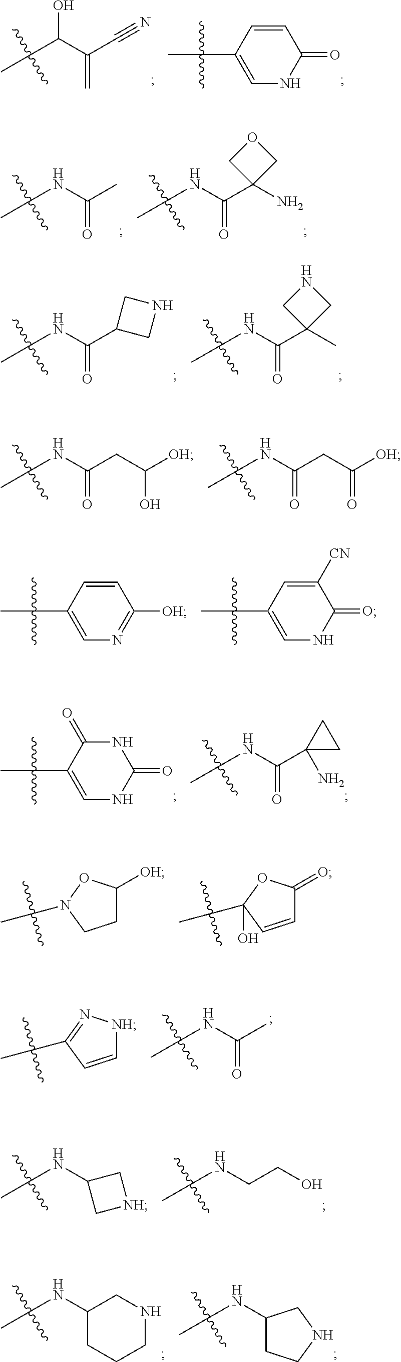

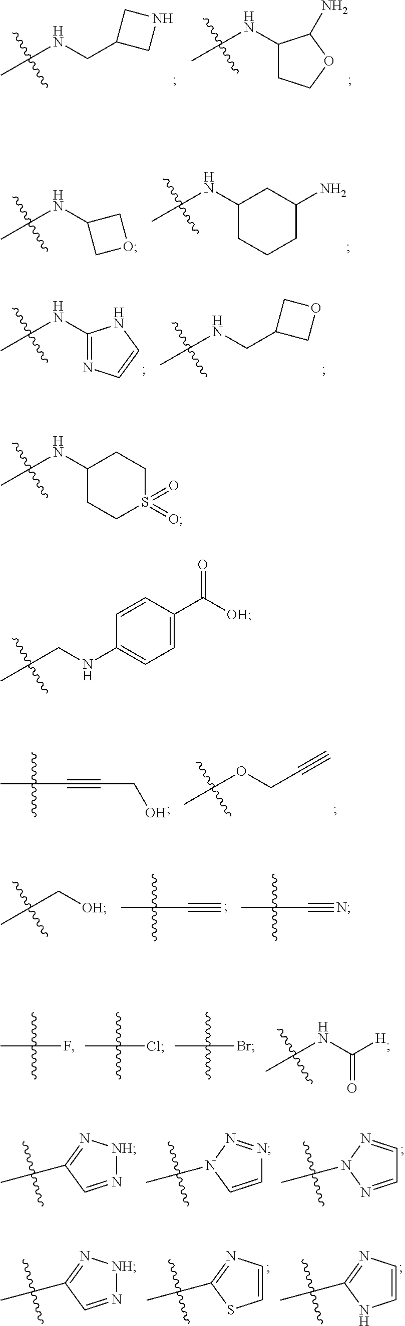

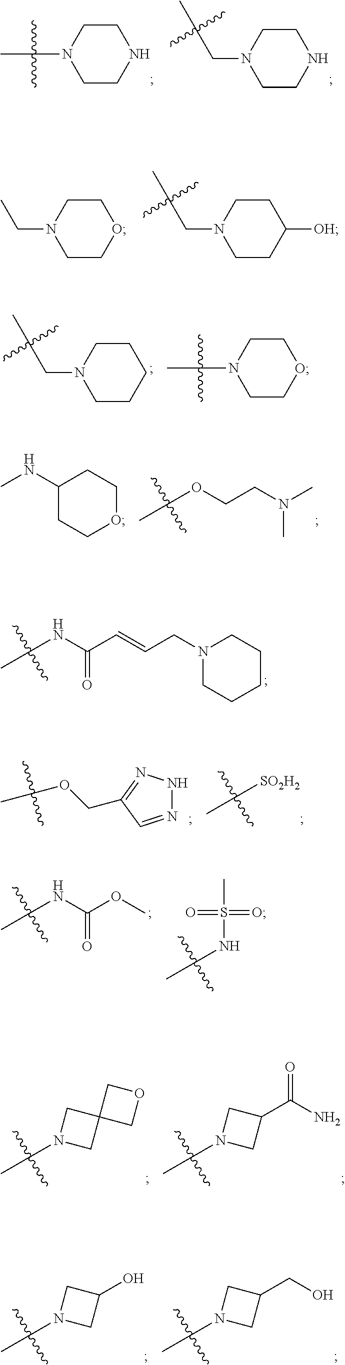

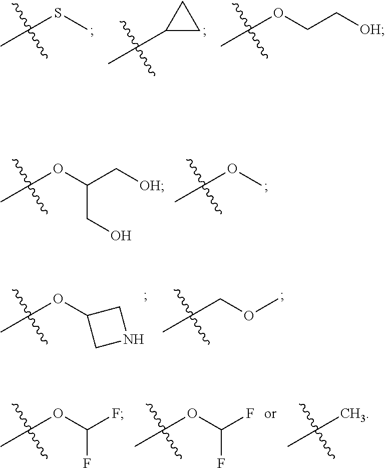

In certain other embodiments, each of R⁶ and R⁷ is H.

In still more embodiments of any of the foregoing embodiments, R⁹, when present, is H. In other embodiments, R⁹, when present, is CH₃.

In other embodiments of any of the foregoing embodiments, R¹⁰, when present, is —OH, methyl, methoxy, nitrilyl, carboxyl or —C(═O)NH₂.

In some embodiments of any of the foregoing embodiments, A is —NH—. In other embodiments A is —NCH₃—. In still other embodiments, A is —O—.

In other embodiments of the foregoing, x is 0. In other embodiments, x is 1. In still more embodiments, y is 0. In other embodiments, y is 1. In still other embodiments, z is 0. In other embodiments, y+z is 1. In certain more embodiments of any of the foregoing, the sum of x+y+z is 1. In other embodiments, the sum of x+y+z is 2.

### Compounds of Formula (Ii), (IV), (V), (VI), (VII), and (VIII)

Suitable groups for A, B, Q, W, R¹, R^{2a}, R^{2b}, R^{3a}, R^{3b}, R^{4a}, R^{4b}, R⁶, R⁷, x, y, and z in compounds of Formula (Ii); Q, W, R¹, R^{2a}, R^{2b}, R^{3a}, R^{3b}, R^{4a}, R^{4b}, R⁶, R⁷, x, y, and z in compounds of Formula (IV) and Formula (V); Q, R¹, R^{2a}, R^{2b}, R^{3a}, R^{3b}, R⁶, R⁷, x, y, and z in compounds of Formula (VI); Q, R¹, R^{2a}, R^{2b}, R^{3a}, R^{3b}, R⁶, x, y, and z in compounds of Formula (VII); and R¹, R^{2a}, R^{2b}, R^{3a}, R^{3b}, R⁶, in compounds of Formula (VIII); are independently selected. The described embodiments of the present invention may be combined. Such combination is contemplated and within the scope of the present invention. For example, it is contemplated that embodiments for any of A, B, Q, W, R¹, R^{2a}, R^{2b}, R^{3a}, R^{3b}, R^{4a}, R^{4b}, R⁶, R⁷, x, y, and z, in compounds of Formula (Ii) can be combined with embodiments defined for any other of A, B, Q, W, R¹, R^{2a}, R^{2b}, R^{3a}, R^{3b}, R^{4a}, R^{4b}, R⁶, R⁷, x, y, and z in compounds of Formula (Ii).

In one aspect, the present invention relates to compounds of Formula (Ii),

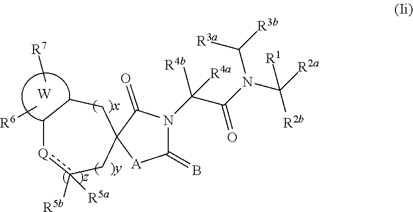

or a stereoisomer, tautomer or pharmaceutically acceptable salt thereof,
wherein:
[symbol] is a single or double bond;
A is —NR⁸—, —O—, or —S—;
B is O or NH;
Q is —C(R¹⁰)ₙ—, —O—, —S(O)ₘ—, —NR⁹—, or —C(O)—;
W is an aryl or heteroaryl ring;
R¹ is carbocyclyl or heterocyclyl;
R^{2a} and R^{2b} are each independently H, D, or C₁-C₆ alkyl;
R^{3a} is hydrogen, C(O)NH₂, C₁-C₆ alkyl, aryl, cycloalkyl or heterocyclyl; and
R^{3b} is C₁-C₆ alkyl, aryl, cycloalkyl or heterocyclyl; or
R^{3a} and R^{3b} taken together with the carbon to which they are attached form aryl, cycloalkyl, or heterocycle;
R^{4a} and R^{4b} are each independently H, D or C₁-C₆ alkyl;
R^{5a} and R^{5b} are each independently H, C₁-C₆ alkyl or absent; or R^{5a} and R^{5b} taken together form oxo;
R⁶ and R⁷ are each independently H, halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹²;
R⁸ and R⁹ are each independently H or C₁-C₆ alkyl;
R¹⁰ is, at each occurrence, independently H, —OH,
   halo, —CN, —CO₂R¹², —C(═O)NHR¹³, —NHR¹², C₁-C₆ alkyl or alkoxy; or two R¹⁰ taken together form oxo or ═N—OR¹¹;
R¹¹ and R¹³ are each independently H, —OH, or C₁-C₆ alkyl;
R¹² is, at each occurrence, independently H, C₁-C₆ alkyl, aryl, cycloalkyl, or heterocyclyl;
n is, at each occurrence, independently 1 or 2;
m is, at each occurrence, independently 0, 1 or 2; and
x, y and z are each independently 0 or 1, wherein x, y and z are selected such that the sum of x+y+z is 1 or 2;

with the proviso that R^{3a} and R^{3b} are not cyclopropyl and methyl, respectively, when R¹ and W are each unsubstituted phenyl, A is —NH, x is 0 or 1, y is 1, z is 0, and Q is —CH₂—; and

with the proviso that R^{3a} and R^{3b} taken together with the carbon to which they are attached do not form tetrahydrothiophene 1,1-dioxide, when R¹ and W are each unsubstituted phenyl, A is —NH, x is 0, y is 1, z is 1, and Q is —O—.

In one embodiment of Formula (Ii), [symbol] is a single or double bond. In another embodiment of Formula (Ii), [symbol] is a single bond. In another embodiment of Formula (Ii), [symbol] is a double bond.

In one embodiment of Formula (Ii), A is —NR⁸—, —O—, or —S—. In another embodiment of Formula (Ii), A is —NR⁸—, or —O—. In another embodiment of Formula (Ii), A is —NR⁸—. In another embodiment of Formula (Ii), A is —O—.

In one embodiment of Formula (Ii), B is O or NH. In another embodiment of Formula (Ii), B is O. In another embodiment of Formula (Ii), B is NH.

In one embodiment of Formula (Ii), Q is —C(R¹⁰)ₙ—, —O—, —S(O)ₘ—, —NR⁹—, or —C(O)—. In another embodiment of Formula (Ii), Q is —C(R¹⁰)ₙ—. In another embodiment of Formula (Ii), Q is —O—. In another embodiment of Formula (Ii), Q is —S(O)ₘ—. In another embodiment of Formula (Ii), Q is —NR⁹—. In another embodiment of Formula (Ii), Q is —C(O)—.

In one embodiment of Formula (Ii), W is an aryl or heteroaryl ring. In another embodiment of Formula (Ii), W is an aryl. In another embodiment of Formula (Ii), W is a heteroaryl ring. In another embodiment of Formula (Ii), W is a phenyl ring. In another embodiment of Formula (Ii), W is pyridinyl, thiophenyl, or pyridinyl-2(1H)-one.

In one embodiment of Formula (Ii), R¹ is carbocyclyl or heterocyclyl. In another embodiment of Formula (Ii), R¹ is carbocyclyl. In another embodiment of Formula (Ii), R¹ is heterocyclyl. In another embodiment of Formula (Ii), R¹ is phenyl. In another embodiment of Formula (Ii), R¹ is phenyl, which is unsubstituted. In another embodiment of Formula (Ii), R¹ is phenyl, which is substituted. In another embodiment of Formula (Ii), R¹ is tetrahydropyranyl, thiazolyl, oxazolyl, thiadiazolyl, pyrimidinyl, thiophenyl, furanyl, triazolyl, or pyridinyl.

In one embodiment of Formula (Ii), R^{2a} and R^{2b} are each independently H, D, or C₁-C₆ alkyl. In another embodiment of Formula (Ii), R^{2a} and R^{2b} are each independently H. In another embodiment of Formula (Ii), R^{2a} is H and R^{2b} is C₁-C₆ alkyl.

In one embodiment of Formula (Ii), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; or R^{3a} and R^{3b} taken together with the carbon to which they are attached form aryl, cycloalkyl, or heterocycle. In another embodiment of Formula (Ii), R^{3a} is C₁-C₆ alkyl; and R^{3b} is C₁-C₆ alkyl; wherein the C₁-C₆ alkyl is optionally substituted with one or more F, —CN, alkoxy, or SO₂R_{g}. In another embodiment of Formula (Ii), R^{3a} is C₁-C₆ alkyl; and R^{3b} is CF₃. In another embodiment of Formula (Ii), R^{3a} is CH₃; and R^{3b} is CF₃. In another embodiment of Formula (Ii), R^{3a} is hydrogen; and R^{3b} is C₁-C₆ alkyl; wherein the C₁-C₆ alkyl is optionally substituted with one or more F, —CN, alkoxy, or SO₂R_{g}. In another embodiment of Formula (Ii), R^{3a} is hydrogen; and R^{3b} is cycloalkyl. In another embodiment of Formula (Ii), R^{3a} is hydrogen; and R^{3b} is cyclopropyl. In another embodiment of Formula (Ii), R^{3a} is C₁-C₆ alkyl; wherein the C₁-C₆ alkyl is; and R^{3b} is cycloalkyl. In another embodiment of Formula (Ii), R^{3a} is CH₃; and R^{3b} is cyclopropyl. In another embodiment of Formula (Ii), R^{3a} is CF₃; and R^{3b} is cyclopropyl. In another embodiment of Formula (Ii), R^{3a} is cycloalkyl; and R^{3b} is cycloalkyl. In another embodiment of Formula (Ii), R^{3a} is cyclopropyl; and R^{3b} is cyclopropyl. In another embodiment of Formula (Ii), R^{3a} is C₁-C₆ alkyl; and R^{3b} is heterocyclyl. In another embodiment of Formula (Ii), R^{3a} is hydrogen; and R^{3b} is aryl. In another embodiment of Formula (Ii), R^{3a} and R^{3b} taken together with the carbon to which they are attached form aryl, cycloalkyl, or heterocycle. In another embodiment of Formula (Ii), R^{3a} and R^{3b} taken together with the carbon to which they are attached form aryl. In another embodiment of Formula (Ii), R^{3a} and R^{3b} taken together with the carbon to which they are attached form cycloalkyl. In another embodiment of Formula (Ii), R^{3a} and R^{3b} taken together with the carbon to which they are attached form heterocycle.

In one embodiment of Formula (Ii), R^{4a} and R^{4b} are each independently H, D or C₁-C₆ alkyl. In another embodiment of Formula (Ii), R^{4a} and R^{4b} are each independently H.

In one embodiment of Formula (Ii), R^{5a} and R^{5b} are each independently H, C₁-C₆ alkyl or absent; or R^{5a} and R^{5b} taken together form oxo. In another embodiment of Formula (Ii), R^{5a} and R^{5b} are each independently H, C₁-C₆ alkyl or absent. In another embodiment of Formula (Ii), R^{5a} and R^{5b} taken together form oxo. In another embodiment of Formula (Ii), R^{5a} and R^{5b} are each independently H. In another embodiment of Formula (Ii), R^{5a} is H and R^{5b} is C₁-C₆ alkyl. In another embodiment of Formula (Ii), R^{5a} and R^{5b} are absent.

In one embodiment of Formula (Ii), R⁶ and R⁷ are each independently H, halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (Ii), R⁷ is H and R⁶ is H, halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (Ii), R⁷ is H and R⁶ is —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (Ii), R⁷ is H and R⁶ is heterocyclyl. In another embodiment of Formula (Ii), R⁷ is H and R⁶ is pyrazolyl. In another embodiment of Formula (Ii), R⁷ is H and R⁶ is —NHC(═O)NHR¹².

Still another embodiment pertains to compounds of Formula (Ii), selected from the group consisting of: Examples 2-1, 3-1, 4-1, 5-1, 6-1, 7-1, 8-1, 9-1, 10-1, 11-1, 12-1, 13-1, 14-1, 15-1, 16-1, 17-1, 18-1, 19-1, 20-1, 21-1, 22-1, 23-1, 24-1, 25-1, 26-1, 27-1, 28-1, 29-1, 30-1, 31-1, 32-1, 33-1, 34-1, 35-1, 36-1, 37-1, 38-1, 39-1, 40-1, 41-1, 42-1, 43-1, 44-1, 45-1, 46-1, 47-1, 48-1, 49-1, 50-1, 51-1, 52-1, 53-1, 54-1, 55-1, 56-1, 57-1, 58-1, 59-1, 60-1, 61-1, 62-1, 63-1, 64-1, 65-1, 66-1, 67-1, 68-1, 69-1, 70-1, 71-1, 72-1, 73-1, 74-1, 75-1, 76-1, 77-1, 77-2, 78-1, 79-1, 80-1, 81-1, 82-1, 83-1, 84-1, 85-1, 86-1, 87-1, 88-1, 89-1, 90-1, 91-1, 92-1, 93-1, 94-1, 95-1, 96-1, 97-1, 98-1, 99-1, 100-1, 101-1, 102-1, 103-1, 104-1, 105-1, 106-1, 107-1, 108-1, 109-1, 110-1, 111-1, 112-1, 113-1, 114-1, 115-1, 116-1, 117-1, 118-1, 119-1, 120-1, 121-1, 122-1, 123-1, 124-1, 125-1, 126-1, 127-1, 128-1, 129-1, 130-1, 131-1, 132-1, 133-1, 134-1, 135-1, 136-1, 137-1, 138-1, 139-1, 140-1, 141-1, 142-1, 143-1, 144-1, 145-1, 146-1, 147-1, 148-1, 149-1, 150-1, 151-1, 152-1, 153-1, 154-1, 155-1, 156-1, 157-1, 158-1, 159-1, 160-1, 161-1, 162-1, 163-1, 164-1, 165-1, 166-1, 167-1, 168-1, 169-1, 170-1, 171-1, 172-1, 173-1, 174-1, 175-1, 176-1, 177-1, 178-1, 179-1, 180-1, 181-1, 182-1, 183-1, 184-1, 185-1, 186-1, 187-1, 188-1, 189-1, 190-1, 191-1, 192-1, 193-1, 194-1, 194-2, 195-1, 196-1, 197-1, 198-1, 199-1, 200-1, 201-1, 202-1, 203-1, 204-1, 205-1, 206-1, 207-1, 208-1, 209-1, 210-1, 211-1, 212-1, 213-1, 214-1, 215-1, 216-1, 217-1, 218-1, 219-1, 220-1, 221-1, 222-1, 223-1, 224-1, 225-1, 226-1, 227-1, 228-1, 229-1, 230-1, 231-1, 232-1, 233-1, 234-1, 235-1, 235-2, 236-1, 237-1, 238-1, 239-1, 240-1, 241-1, 242-1, 243-1, 244-1, 245-1, 246-1, 247-1, 248-1, 249-1, 250-1, 251-1, 252-1, 253-1, 254-1, 255-1, 256-1, 257-1, 258-1, 259-1, 260-1, 261-1, 261-2, 262-1, 263-1, 264-1, 265-1, 265-2, 266-1, 266-2, 267-1, 267-2, 268-1, 268-2, 268-3, 268-4, 269-1, 269-2, 270-1, 270-2, 271-1, 271-2, 272-1, 272-2, 273-1, 273-2, 274-1, 275-1, 275-2, 276-1, 277-1, 278-1, 279-1, 280-1, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, and pharmaceutically acceptable salts thereof.

In another aspect, the present invention relates to compounds of Formula (IV),

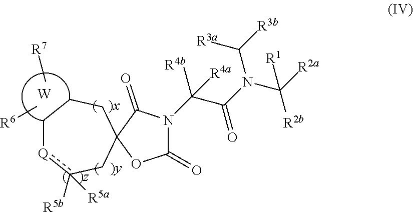

or a stereoisomer, tautomer or pharmaceutically acceptable salt thereof, wherein:
[symbol] is a single or double bond;
Q is —C(R¹⁰)ₙ—, —O—, —S(O)ₘ—, —NR⁹—, or —C(O)—;
W is an aryl or heteroaryl ring;
R¹ is carbocyclyl or heterocyclyl;
R^{2a} and R^{2b} are each independently H, D, or C₁-C₆ alkyl;
R^{3a} is hydrogen, C(O)NH₂, C₁-C₆ alkyl, aryl, cycloalkyl or heterocyclyl; and
R^{3b} is C₁-C₆ alkyl, aryl, cycloalkyl or heterocyclyl; or
R^{3a} and R^{3b} taken together with the carbon to which they are attached form aryl, cycloalkyl, or heterocycle;
R^{4a} and R^{4b} are each independently H, D or C₁-C₆ alkyl;
R^{5a} and R^{5b} are each independently H, C₁-C₆ alkyl or absent; or R^{5a} and R^{5b} taken together form oxo;
R⁶ and R⁷ are each independently H, halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹²;
R⁸ and R⁹ are each independently H or C₁-C₆ alkyl;
R¹⁰ is, at each occurrence, independently H, OH, halo, —CN, —CO₂R¹², —C(═O)NHR¹³, —NHR¹², C₁-C₆ alkyl or alkoxy; or two R¹⁰ taken together form oxo or ═N—OR¹¹;
R¹¹ and R¹³ are each independently H, —OH or C₁-C₆ alkyl;
R¹² is, at each occurrence, independently H, C₁-C₆ alkyl, aryl, cycloalkyl, or heterocyclyl;
n is, at each occurrence, independently 1 or 2;
m is, at each occurrence, independently 0, 1 or 2; and
x, y and z are each independently 0 or 1, wherein x, y and z are selected such that the sum of x+y+z is 1 or 2.

In one embodiment of Formula (IV), [symbol] is a single or double bond. In another embodiment of Formula (IV), [symbol] is a single bond. In another embodiment of Formula (IV), [symbol] is a double bond.

In one embodiment of Formula (IV), Q is —C(R¹⁰)ₙ—, —O—, —S(O)ₘ—, —NR⁹—, or —C(O)—. In another embodiment of Formula (IV), Q is —C(R¹⁰)ₙ—. In another embodiment of Formula (IV), Q is —O—. In another embodiment of Formula (IV), Q is —S(O)ₘ—. In another embodiment of Formula (IV), Q is —NR⁹—. In another embodiment of Formula (IV), Q is —C(O)—.

In one embodiment of Formula (IV), W is an aryl or heteroaryl ring. In another embodiment of Formula (IV), W is an aryl. In another embodiment of Formula (IV), W is a heteroaryl ring. In another embodiment of Formula (IV), W is a phenyl ring. In another embodiment of Formula (IV), W is pyridinyl, thiophenyl, or pyridinyl-2(1H)-one.

In one embodiment of Formula (IV), R¹ is carbocyclyl or heterocyclyl. In another embodiment of Formula (IV), R¹ is carbocyclyl. In another embodiment of Formula (IV), R¹ is heterocyclyl. In another embodiment of Formula (IV), R¹ is phenyl. In another embodiment of Formula (IV), R¹ is phenyl, which is unsubstituted. In another embodiment of Formula (IV), R¹ is phenyl, which is substituted. In another embodiment of Formula (IV), R¹ is tetrahydropyranyl, thiazolyl, oxazolyl, thiadiazolyl, pyrimidinyl, thiophenyl, furanyl, triazolyl, or pyridinyl.

In one embodiment of Formula (IV), R^{2a} and R^{2b} are each independently H, D, or C₁-C₆ alkyl. In another embodiment of Formula (IV), R^{2a} and R^{2b} are each independently H. In another embodiment of Formula (IV), R^{2a} is H and R^{2b} is C₁-C₆ alkyl.

In one embodiment of Formula (IV), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; or R^{3a} and R^{3b} taken together with the carbon to which they are attached form aryl, cycloalkyl, or heterocycle. In another embodiment of Formula (IV), R^{3a} is C₁-C₆ alkyl; and R^{3b} is C₁-C₆ alkyl; wherein the C₁-C₆ alkyl is optionally substituted with one or more F, C—N, alkoxy, or SO₂R_{g}. In another embodiment of Formula (IV), R^{3a} is CH₃; and R^{3b} is CF₃. In another embodiment of Formula (IV), R^{3a} is hydrogen; and R^{3b} is C₁-C₆ alkyl; wherein the C₁-C₆ alkyl is optionally substituted with F, —CN, alkoxy, or SO₂R_{g}. In another embodiment of Formula (IV), R^{3a} is hydrogen; and R^{3b} is cycloalkyl.

In another embodiment of Formula (IV), R^{3a} is hydrogen; and R^{3b} is cyclopropyl. In another embodiment of Formula (IV), R^{3a} is C₁-C₆ alkyl; wherein the C₁-C₆ alkyl is optionally substituted with one or more F; and R^{3b} is cycloalkyl. In another embodiment of Formula (IV), R^{3a} is CH₃; and R^{3b} is cyclopropyl. In another embodiment of Formula (IV), R^{3a} is CF₃; and R^{3b} is cyclopropyl. In another embodiment of Formula (IV), R^{3a} is cycloalkyl; and R^{3b} is cycloalkyl. In another embodiment of Formula (IV), R^{3a} is cyclopropyl; and R^{3b} is cyclopropyl. In another embodiment of Formula (IV), R^{3a} is C₁-C₆ alkyl; and R^{3b} is heterocyclyl. In another embodiment of Formula (IV), R^{3a} is hydrogen; and R^{3b} is aryl. In another embodiment of Formula (IV), R^{3a} and R^{3b} taken together with the carbon to which they are attached form aryl, cycloalkyl, or heterocycle. In another embodiment of Formula (IV), R^{3a} and R^{3b} taken together with the carbon to which they are attached form aryl. In another embodiment of Formula (IV), R^{3a} and R^{3b} taken together with the carbon to which they are attached form cycloalkyl. In another embodiment of Formula (IV), R^{3a} and R^{3b} taken together with the carbon to which they are attached form heterocycle.

In one embodiment of Formula (IV), R^{4a} and R^{4b} are each independently H, D or C₁-C₆ alkyl. In another embodiment of Formula (IV), R^{4a} and R^{4b} are each independently H.

In one embodiment of Formula (IV), R^{5a} and R^{5b} are each independently H, C₁-C₆ alkyl or absent; or R^{5a} and R^{5b} taken together form oxo. In another embodiment of Formula (IV), R^{5a} and R^{5b} are each independently H, C₁-C₆ alkyl or absent. In another embodiment of Formula (IV), R^{5a} and R^{5b} taken together form oxo. In another embodiment of Formula (IV), R^{5a} and R^{5b} are each independently H. In another embodiment of Formula (IV), R^{5a} is H and R^{5b} is C₁-C₆ alkyl. In another embodiment of Formula (IV), R^{5a} and R^{5b} are absent.

In one embodiment of Formula (IV), R⁶ and R⁷ are each independently H, halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (IV), R⁷ is H and R⁶ is H, halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (IV), R⁷ is H and R⁶ is —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (IV), R⁷ is H and R⁶ is heterocyclyl. In another embodiment of Formula (IV), R⁷ is H and R⁶ is pyrazolyl. In another embodiment of Formula (IV), R⁷ is H and R⁶ is —NHC(═O)NHR¹².

Still another embodiment pertains to compounds of Formula (IV), selected from the group consisting of: Examples 43-1, 71-1, 72-1, 73-1, 74-1, 75-1, 88-1, 89-1, 96-1, 97-1, 100-1, 104-1, 108-1, 113-1, 117-1, 118-1, 121-1, 122-1, 123-1, 127-1, 128-1, 129-1, 130-1, 131-1, 132-1, 138-1, 139-1, 140-1, 143-1, 144-1, 147-1, 148-1, 151-1, 152-1, 153-1, 154-1, 157-1, 158-1, 159-1, 160-1, 161-1, 162-1, 163-1, 164-1, 165-1, 167-1, 169-1, 171-1, 172-1, 174-1, 175-1, 176-1, 177-1, 178-1, 179-1, 181-1, 184-1, 185-1, 186-1, 191-1, 192-1, 195-1, 198-1, 199-1, 200-1, 201-1, 202-1, 203-1, 204-1, 206-1, 208-1, 209-1, 210-1, 211-1, 212-1, 213-1, 214-1, 215-1, 216-1, 217-1, 218-1, 219-1, 220-1, 221-1, 222-1, 223-1, 224-1, 225-1, 226-1, 227-1, 228-1, 229-1, 230-1, 231-1, 232-1, 233-1, 234-1, 235-1, 235-2, 236-1, 237-1, 238-1, 242-1, 243-1, 245-1, 249-1, 250-1, 251-1, 258-1, 259-1, 260-1, 261-1, 261-2, 262-1, 263-1, 264-1, 265-1, 265-2, 266-1, 266-2, 267-1, 267-2, 268-1, 268-2, 268-3, 268-4, 269-1, 269-2, 270-1, 270-2, 271-1, 271-2, 272-1, 272-2, 273-1, 273-2, 274-1, 275-1, 275-2, 276-1, 277-1, 278-1, 279-1, 280-1, 281, 282, 283, 284, 285, 286, 287, 288, 291, 292, 293, 294, 297, 298, 299, 301, 302, 303, 304, 305, 306, 307, 308, 309, 310, 311, 312, 313, 314, 315, 316, 317, 318, 319, 320, 321, 324, 325, 326, 327, 328, 329, 330, 331, 332, 333, 334, 335, 336, 337, 338, 339, 340, 341, 342, 343, 344, 345, 349, 350, 352, 353, 354, 359, 361, 362, 363, 364, 365, 367, 368, 369, 370, 371, 372, 373, 374, 375, 377, 378, 382, 383, 385, 387, 388, 389, 392, 393, 394, 395, 396, 397, 398, 399, 400, 401, 402, 403, 404, 405, 406, 407, 408, 409, 411, 412, 413, 414, 415, 416, 417, 418, 419, 420, 421, 422, 424, 426, 427, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 490, 494, 496, 497, 498, 502, 503, 504, 506, 507, 508, 509, 511, 512, 516, 517, 519, 520, 521, 523, 524, 525, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 565, 566, 567, 568, 572, 573, 574, 575, 576, 577, 578, 581, 583, 584, 585, 586, 587, 588, 589, 590, 591, 592, 593, 594, 595, 596, 599, 600, 601, 602, 603, 604, 606, 607, 608, 609, 610, 611, 612, 613, 614, 615, 616, 622, 624, 626, 627, 628, 631, 632, 633, 634, 635, 636, 639, 642, 648, 649, 651, 652, 653, 654, 656, 658, 659, 660, 661, 662, 663, 664, 665, 666, 668, 669, 670, 671, 672, 674, 675, 676, 677, 678, 684, 687, 688, 689, 694, 695, 696, 697, 702, 707, 708, 709, 710, 712, 713, 714, 718, 719, 720, 721, 722, 723, 725, 726, 727, 728, 729, 730, 732, 733, 734, 736, 737, 752, 754, 755, 756, 757, 758, and pharmaceutically acceptable salts thereof.

In another aspect, the present invention relates to compounds of Formula (V),

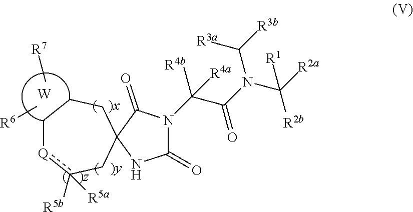

or a stereoisomer, tautomer or pharmaceutically acceptable salt thereof,
wherein:
[symbol] is a single or double bond;
Q is —C(R¹⁰)ₙ—, —O—, —S(O)ₘ—, —NR⁹—, or —C(O)—;
W is an aryl or heteroaryl ring;
R¹ is carbocyclyl or heterocyclyl;
R^{2a} and R^{2b} are each independently H, D, or C₁-C₆ alkyl;
R^{3a} is hydrogen, C(O)NH₂, C₁-C₆ alkyl, aryl, cycloalkyl or heterocyclyl; and
R^{3b} is C₁-C₆ alkyl, aryl, cycloalkyl or heterocyclyl; or
R^{3a} and R^{3b} taken together with the carbon to which they are attached form aryl, cycloalkyl, or heterocycle;
R^{4a} and R^{4b} are each independently H, D or C₁-C₆ alkyl;
R^{5a} and R^{5b} are each independently H, C₁-C₆ alkyl or absent; or R^{5a} and R^{5b} taken together form oxo;
R⁶ and R⁷ are each independently H, halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹²;
R⁸ and R⁹ are each independently H or C₁-C₆ alkyl;
R¹⁰ is, at each occurrence, independently H, OH, halo, —CN, —CO₂R¹², —C(═O)NHR¹³, —NHR¹², C₁-C₆ alkyl or alkoxy; or two R¹⁰ taken together form oxo or ═N—OR¹¹;
R¹¹ and R¹³ are each independently H, —OH or C₁-C₆ alkyl;
R¹² is, at each occurrence, independently H, C₁-C₆ alkyl, aryl, cycloalkyl, or heterocyclyl;
n is, at each occurrence, independently 1 or 2;
m is, at each occurrence, independently 0, 1 or 2; and
x, y and z are each independently 0 or 1, wherein x, y and z are selected such that the sum of x+y+z is 1 or 2;

with the proviso that R^{3a} and R^{3b} are not cyclopropyl and methyl, respectively, when R¹ and W are each unsubstituted phenyl, is 0 or 1, y is 1, z is 0, and Q is —CH₂—; and

with the proviso that R^{3a} and R^{3b} taken together with the carbon to which they are attached do not form tetrahydrothiophene 1,1-dioxide, when R¹ and W are each unsubstituted phenyl, is 0, y is 1, z is 1, and Q is —O—.

In one embodiment of Formula (V), [symbol] is a single or double bond. In another embodiment of Formula (V), [symbol] is a single bond. In another embodiment of Formula (V), [symbol] is a double bond.

In one embodiment of Formula (V), Q is —C(R¹⁰)ₙ—, —O—, —S(O)ₘ—, —NR⁹—, or —C(O)—. In another embodiment of Formula (V), Q is —C(R¹⁰)ₙ—. In another embodiment of Formula (V), Q is —O—. In another embodiment of Formula (V), Q is —S(O)ₘ—. In another embodiment of Formula (V), Q is —NR⁹—. In another embodiment of Formula (V), Q is —C(O)—.

In one embodiment of Formula (V), W is an aryl or heteroaryl ring. In another embodiment of Formula (V), W is an aryl. In another embodiment of Formula (V), W is a heteroaryl ring. In another embodiment of Formula (V), W is a phenyl ring. In another embodiment of Formula (V), W is pyridinyl, thiophenyl, or pyridinyl-2(1H)-one.

In one embodiment of Formula (V), R¹ is carbocyclyl or heterocyclyl. In another embodiment of Formula (V), R¹ is carbocyclyl. In another embodiment of Formula (V), R¹ is heterocyclyl. In another embodiment of Formula (V), R¹ is phenyl. In another embodiment of Formula (V), R¹ is phenyl, which is unsubstituted. In another embodiment of Formula (V), R¹ is phenyl, which is substituted. In another embodiment of Formula (V), R¹ is tetrahydropyranyl, thiazolyl, oxazolyl, thiadiazolyl, pyrimidinyl, thiophenyl, furanyl, triazolyl, or pyridinyl.

In one embodiment of Formula (V), R^{2a} and R^{2b} are each independently H, D, or C₁-C₆ alkyl. In another embodiment of Formula (V), R^{2a} and R^{2b} are each independently H. In another embodiment of Formula (V), R^{2a} is H and R^{2b} is C₁-C₆ alkyl.

In one embodiment of Formula (V), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; or R^{3a} and R^{3b} taken together with the carbon to which they are attached form aryl, cycloalkyl, or heterocycle. In another embodiment of Formula (V), R^{3a} is C₁-C₆ alkyl; and R^{3b} is C₁-C₆ alkyl; wherein the C₁-C₆ alkyl is optionally substituted with one or more F, —CN, alkoxy, or SO₂R_{g}. In another embodiment of Formula (V), R^{3a} is C₁-C₆ alkyl; and R^{3b} is CF₃. In another embodiment of Formula (V), R^{3a} is CH₃; and R^{3b} is CF₃. In another embodiment of Formula (V), R^{3a} is hydrogen; and R^{3b} is C₁-C₆ alkyl; wherein the C₁-C₆ alkyl is optionally substituted with one or more F, —CN, alkoxy, or SO₂R_{g}. In another embodiment of Formula (V), R^{3a} is hydrogen; and R^{3b} is cycloalkyl. In another embodiment of Formula (V), R^{3a} is hydrogen; and R^{3b} is cyclopropyl. In another embodiment of Formula (V), R^{3a} is C₁-C₆ alkyl; wherein the C₁-C₆ alkyl is optionally substituted with one or more F; and R^{3b} is cycloalkyl. In another embodiment of Formula (V), R^{3a} is CH₃; and R^{3b} is cyclopropyl. In another embodiment of Formula (V), R^{3a} is CF₃; and R^{3b} is cyclopropyl. In another embodiment of Formula (V), R^{3a} is cycloalkyl; and R^{3b} is cycloalkyl. In another embodiment of Formula (V), R^{3a} is cyclopropyl; and R^{3b} is cyclopropyl. In another embodiment of Formula (V), R^{3a} is C₁-C₆ alkyl; and R^{3b} is heterocyclyl. In another embodiment of Formula (V), R^{3a} is hydrogen; and R^{3b} is aryl. In another embodiment of Formula (V), R^{3a} and R^{3b} taken together with the carbon to which they are attached form aryl, cycloalkyl, or heterocycle. In another embodiment of Formula (V), R^{3a} and R^{3b} taken together with the carbon to which they are attached form aryl. In another embodiment of Formula (V), R^{3a} and R^{3b} taken together with the carbon to which they are attached form cycloalkyl. In another embodiment of Formula (V), R^{3a} and R^{3b} taken together with the carbon to which they are attached form heterocycle.

In one embodiment of Formula (V), R^{4a} and R^{4b} are each independently H, D or C₁-C₆ alkyl. In another embodiment of Formula (V), R^{4a} and R^{4b} are each independently H.

In one embodiment of Formula (V), R^{5a} and R^{5b} are each independently H, C₁-C₆ alkyl or absent; or R^{5a} and R^{5b} taken together form oxo. In another embodiment of Formula (V), R^{5a} and R^{5b} are each independently H, C₁-C₆ alkyl or absent. In another embodiment of Formula (V), R^{5a} and R^{5b} taken together form oxo. In another embodiment of Formula (V), R^{5a} and R^{5b} are each independently H. In another embodiment of Formula (V), R^{5a} is H and R^{5b} is C₁-C₆ alkyl. In another embodiment of Formula (V), R^{5a} and R^{5b} are absent.

In one embodiment of Formula (V), R⁶ and R⁷ are each independently H, halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (V), R⁷ is H and R⁶ is H, halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (V), R⁷ is H and R⁶ is —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (V), R⁷ is H and R⁶ is heterocyclyl. In another embodiment of Formula (V), R⁷ is H and R⁶ is pyrazolyl. In another embodiment of Formula (V), R⁷ is H and R⁶ is —NHC(═O)NHR¹².

Still another embodiment pertains to compounds of Formula (V), selected from the group consisting of: Examples 2-1, 3-1, 4-1, 5-1, 6-1, 7-1, 8-1, 9-1, 10-1, 11-1, 12-1, 13-1, 14-1, 15-1, 16-1, 17-1, 18-1, 19-1, 20-1, 21-1, 22-1, 23-1, 24-1, 25-1, 26-1, 27-1, 28-1, 29-1, 30-1, 31-1, 32-1, 33-1, 34-1, 35-1, 36-1, 37-1, 38-1, 39-1, 40-1, 41-1, 42-1, 44-1, 45-1, 46-1, 47-1, 48-1, 49-1, 50-1, 51-1, 52-1, 53-1, 54-1, 55-1, 56-1, 57-1, 58-1, 59-1, 61-1, 62-1, 63-1, 64-1, 65-1, 66-1, 67-1, 68-1, 69-1, 70-1, 76-1, 77-1, 77-2, 78-1, 79-1, 80-1, 81-1, 82-1, 83-1, 84-1, 85-1, 86-1, 87-1, 90-1, 91-1, 92-1, 93-1, 94-1, 95-1, 98-1, 99-1, 101-1, 102-1, 103-1, 105-1, 106-1, 107-1, 109-1, 110-1, 111-1, 112-1, 114-1, 115-1, 116-1, 119-1, 120-1, 124-1, 125-1, 126-1, 133-1, 134-1, 135-1, 136-1, 137-1, 141-1, 142-1, 145-1, 146-1, 149-1, 150-1, 155-1, 156-1, 166-1, 168-1, 170-1, 173-1, 180-1, 182-1, 183-1, 187-1, 188-1, 189-1, 190-1, 193-1, 194-1, 194-2, 196-1, 197-1, 205-1, 207-1, 239-1, 240-1, 241-1, 244-1, 246-1, 247-1, 248-1, 252-1, 253-1, 254-1, 255-1, 256-1, 257-1, 289, 290, 295, 296, 300, 322, 323, 346, 347, 348, 351, 355, 356, 357, 358, 360, 366, 376, 379, 380, 381, 384, 386, 390, 391, 410, 423, 425, 428, 486, 487, 488, 489, 491, 492, 493, 495, 499, 500, 501, 505, 510, 513, 514, 515, 518, 522, 526, 527, 528, 563, 564, 569, 570, 571, 579, 580, 582, 597, 598, 605, 617, 618, 619, 620, 621, 623, 625, 629, 630, 637, 638, 640, 641, 643, 644, 645, 646, 647, 650, 655, 657, 667, 673, 679, 680, 681, 682, 683, 685, 686, 690, 691, 692, 693, 698, 699, 700, 701, 703, 704, 705, 706, 711, 715, 716, 717, 724, 731, 735, 738, 739, 740, 741, 742, 743, 744, 745, 746, 747, 748, 749, 750, 751, 753, 759, and pharmaceutically acceptable salts thereof.

In another aspect, the present invention relates to compounds of Formula (VI),

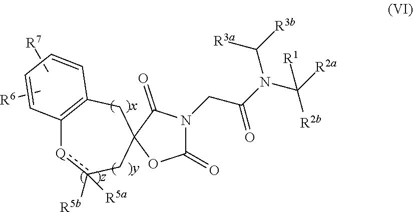

or a stereoisomer, tautomer or pharmaceutically acceptable salt thereof,
wherein:
[symbol] is a single or double bond;
Q is —C(R¹⁰)ₙ—, —O—, —S(O)ₘ—, —NR⁹—, or —C(O)—;
R¹ is carbocyclyl or heterocyclyl;
R^{2a} and R^{2b} are each independently H, D, or C₁-C₆ alkyl;
R^{3a} is hydrogen, C(O)NH₂, C₁-C₆ alkyl, aryl, cycloalkyl or heterocyclyl; and
R^{3b} is C₁-C₆ alkyl, aryl, cycloalkyl or heterocyclyl; or
R^{3a} and R^{3b} taken together with the carbon to which they are attached form aryl, cycloalkyl, or heterocycle;
R^{5a} and R^{5b} are each independently H, C₁-C₆ alkyl or absent; or R^{5a} and R^{5b} taken together form oxo;
R⁶ and R⁷ are each independently H, halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹²;
R⁸ and R⁹ are each independently H or C₁-C₆ alkyl;
R¹⁰ is, at each occurrence, independently H, —OH, halo, —CN, —CO₂R¹², —C(═O)NHR¹³, —NHR¹², C₁-C₆ alkyl or alkoxy; or two R¹⁰ taken together form oxo or ═N—OR¹¹;
R¹¹ and R¹³ are each independently H, —OH or C₁-C₆ alkyl;
R¹² is, at each occurrence, independently H, C₁-C₆ alkyl, aryl, cycloalkyl, or heterocyclyl;
n is, at each occurrence, independently 1 or 2;
m is, at each occurrence, independently 0, 1 or 2; and
x, y and z are each independently 0 or 1, wherein x, y and z are selected such that the sum of x+y+z is 1 or 2.

In one embodiment of Formula (VI), [symbol] is a single or double bond. In another embodiment of Formula (VI), [symbol] is a single bond. In another embodiment of Formula (VI), [symbol] is a double bond.

In one embodiment of Formula (VI), Q is —C(R¹⁰)ₙ—, —O—, —S(O)ₘ—, —NR⁹—, or —C(O)—. In another embodiment of Formula (VI), Q is —C(R¹⁰)ₙ—. In another embodiment of Formula (VI), Q is —O—. In another embodiment of Formula (VI), Q is —S(O)ₘ—. In another embodiment of Formula (VI), Q is —NR⁹—. In another embodiment of Formula (VI), Q is —C(O)—.

In one embodiment of Formula (VI), R¹ is carbocyclyl or heterocyclyl. In another embodiment of Formula (VI), R¹ is carbocyclyl. In another embodiment of Formula (VI), R¹ is heterocyclyl. In another embodiment of Formula (VI), R¹ is phenyl. In another embodiment of Formula (VI), R¹ is phenyl, which is unsubstituted. In another embodiment of Formula (VI), R¹ is phenyl, which is substituted. In another embodiment of Formula (VI), R¹ is tetrahydropyranyl, thiazolyl, oxazolyl, thiadiazolyl, pyrimidinyl, thiophenyl, furanyl, triazolyl, or pyridinyl.

In one embodiment of Formula (VI), R^{2a} and R^{2b} are each independently H, D, or C₁-C₆ alkyl. In another embodiment of Formula (VI), R^{2a} and R^{2b} are each independently H. In another embodiment of Formula (VI), R^{2a} is H and R^{2b} is C₁-C₆ alkyl.

In one embodiment of Formula (VI), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; or R^{3a} and R^{3b} taken together with the carbon to which they are attached form aryl, cycloalkyl, or heterocycle. In another embodiment of Formula (VI), R^{3a} is C₁-C₆ alkyl; and R^{3b} is C₁-C₆ alkyl; wherein the C₁-C₆ alkyl is optionally substituted with one or more F, —CN, alkoxy, or SO₂R_{g}. In another embodiment of Formula (VI), R^{3a} is C₁-C₆ alkyl; and R^{3b} is CF₃. In another embodiment of Formula (VI), R^{3a} is CH₃; and R^{3b} is CF₃. In another embodiment of Formula (VI), R^{3a} is hydrogen; and R^{3b} is C₁-C₆ alkyl; wherein the C₁-C₆ alkyl is optionally substituted with one or more F, —CN, alkoxy, or SO₂R_{g}. In another embodiment of Formula (VI), R^{3a} is hydrogen; and R^{3b} is cycloalkyl. In another embodiment of Formula (VI), R^{3a} is hydrogen; and R^{3b} is cyclopropyl. In another embodiment of Formula (VI), R^{3a} is C₁-C₆ alkyl; wherein the C₁-C₆ alkyl is optionally substituted with one or more F; and R^{3b} is cycloalkyl. In another embodiment of Formula (VI), R^{3a} is CH₃; and R^{3b} is cyclopropyl. In another embodiment of Formula (VI), R^{3a} is CF₃; and R^{3b} is cyclopropyl. In another embodiment of Formula (VI), R^{3a} is cycloalkyl; and R^{3b} is cycloalkyl. In another embodiment of Formula (VI), R^{3a} is cyclopropyl; and R^{3b} is cyclopropyl. In another embodiment of Formula (VI), R^{3a} is C₁-C₆ alkyl; and R^{3b} is heterocyclyl. In another embodiment of Formula (VI), R^{3a} is hydrogen; and R^{3b} is aryl. In another embodiment of Formula (VI), R^{3a} and R^{3b} taken together with the carbon to which they are attached form aryl, cycloalkyl, or heterocycle. In another embodiment of Formula (VI), R^{3a} and R^{3b} taken together with the carbon to which they are attached form aryl. In another embodiment of Formula (VI), R^{3a} and R^{3b} taken together with the carbon to which they are attached form cycloalkyl. In another embodiment of Formula (VI), R^{3a} and R^{3b} taken together with the carbon to which they are attached form heterocycle.

In one embodiment of Formula (VI), R^{5a} and R^{5b} are each independently H, C₁-C₆ alkyl or absent; or R^{5a} and R^{5b} taken together form oxo. In another embodiment of Formula (VI), R^{5a} and R^{5b} are each independently H, C₁-C₆ alkyl or absent. In another embodiment of Formula (VI), R^{5a} and R^{5b} taken together form oxo. In another embodiment of Formula (VI), R^{5a} and R^{5b} are each independently H. In another embodiment of Formula (VI), R^{5a} is H and R^{5b} is C₁-C₆ alkyl. In another embodiment of Formula (VI), R^{5a} and R^{5b} are absent.

In one embodiment of Formula (VI), R⁶ and R⁷ are each independently H, halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (VI), R⁷ is H and R⁶ is H, halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (VI), R⁶ is H and R⁶ is —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (VI), R⁷ is H and R⁶ is heterocyclyl. In another embodiment of Formula (VI), R⁷ is H and R⁶ is pyrazolyl. In another embodiment of Formula (VI), R⁷ is H and R⁶ is —NHC(═O)NHR¹².

Still another embodiment pertains to compounds of Formula (VI), selected from the group consisting of: Examples 43-1, 71-1, 72-1, 73-1, 74-1, 75-1, 88-1, 89-1, 96-1, 97-1, 100-1, 104-1, 108-1, 113-1, 117-1, 118-1, 121-1, 122-1, 123-1, 127-1, 128-1, 129-1, 130-1, 131-1, 132-1, 138-1, 139-1, 140-1, 143-1, 144-1, 147-1, 148-1, 151-1, 152-1, 153-1, 154-1, 157-1, 158-1, 159-1, 160-1, 161-1, 162-1, 163-1, 164-1, 165-1, 167-1, 169-1, 171-1, 172-1, 174-1, 175-1, 176-1, 177-1, 178-1, 179-1, 181-1, 184-1, 185-1, 186-1, 191-1, 192-1, 195-1, 198-1, 199-1, 200-1, 201-1, 202-1, 203-1, 204-1, 206-1, 208-1, 209-1, 210-1, 211-1, 212-1, 213-1, 214-1, 215-1, 216-1, 217-1, 218-1, 219-1, 220-1, 221-1, 222-1, 223-1, 224-1, 225-1, 226-1, 227-1, 228-1, 229-1, 230-1, 231-1, 232-1, 233-1, 234-1, 235-1, 235-2, 236-1, 237-1, 238-1, 242-1, 243-1, 245-1, 249-1, 250-1, 251-1, 258-1, 259-1, 260-1, 261-1, 261-2, 262-1, 263-1, 264-1, 265-1, 265-2, 266-1, 266-2, 267-1, 267-2, 268-1, 268-2, 268-3, 268-4, 269-1, 269-2, 270-1, 270-2, 271-1, 271-2, 272-1, 272-2, 273-1, 273-2, 274-1, 275-1, 275-2, 276-1, 277-1, 278-1, 279-1, 280-1, 281, 282, 283, 284, 285, 286, 287, 288, 291, 292, 293, 294, 297, 298, 299, 301, 302, 303, 304, 305, 306, 307, 308, 309, 310, 311, 312, 313, 314, 315, 316, 317, 318, 319, 320, 321, 324, 325, 326, 327, 328, 329, 330, 331, 332, 333, 334, 335, 336, 337, 338, 339, 340, 341, 342, 343, 344, 345, 349, 350, 352, 353, 354, 359, 361, 362, 363, 364, 365, 367, 368, 369, 370, 371, 372, 373, 374, 375, 377, 378, 382, 383, 385, 387, 388, 389, 392, 393, 394, 395, 396, 397, 398, 399, 400, 401, 402, 403, 404, 405, 406, 407, 408, 409, 411, 412, 413, 414, 415, 416, 417, 418, 419, 420, 421, 422, 424, 426, 427, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 490, 494, 496, 497, 498, 502, 503, 504, 506, 507, 508, 509, 511, 512, 516, 517, 519, 520, 521, 523, 524, 525, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 565, 566, 567, 568, 572, 573, 574, 575, 576, 577, 578, 581, 583, 584, 585, 586, 587, 588, 589, 590, 591, 592, 593, 594, 595, 596, 599, 600, 601, 602, 603, 604, 606, 607, 608, 609, 610, 611, 612, 613, 614, 615, 616, 622, 624, 626, 627, 628, 631, 632, 633, 634, 635, 636, 639, 642, 648, 649, 651, 652, 653, 654, 656, 658, 659, 660, 661, 662, 663, 664, 665, 666, 668, 669, 670, 671, 672, 674, 675, 676, 677, 678, 684, 687, 688, 689, 694, 695, 696, 697, 702, 707, 708, 709, 710, 712, 713, 714, 718, 719, 720, 721, 722, 723, 725, 726, 727, 728, 729, 730, 732, 733, 734, 736, 737, 752, 754, 755, 756, 757, 758, and pharmaceutically acceptable salts thereof.

In another aspect, the present invention relates to compounds of Formula (VII),

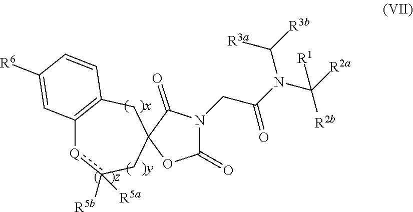

or a stereoisomer, tautomer or pharmaceutically acceptable salt thereof,
wherein:
[symbol] is a single or double bond;
Q is —C(R¹⁰)ₙ—, —O—, —S(O)ₘ—, —NR⁹—, or —C(O)—;
R¹ is carbocyclyl or heterocyclyl;
R^{2a} and R^{2b} are each independently H, D, or C₁-C₆ alkyl;
R^{3a} is hydrogen, C(O)NH₂, C₁-C₆ alkyl, aryl, cycloalkyl or heterocyclyl; and
R^{3b} is C₁-C₆ alkyl, aryl, cycloalkyl or heterocyclyl; or
R^{3a} and R^{3b} taken together with the carbon to which they are attached form aryl, cycloalkyl, or heterocycle;
R^{5a} and R^{5b} are each independently H, C₁-C₆ alkyl or absent; or R^{5a} and R^{5b} taken together form oxo;
R⁶ is independently halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹²;
R⁸ and R⁹ are each independently H or C₁-C₆ alkyl;
R¹⁰ is, at each occurrence, independently H, —OH, halo, —CN, —CO₂R¹², —C(═O)NHR¹³, —NHR¹², C₁-C₆ alkyl or alkoxy; or two R¹⁰ taken together form oxo or ═N—OR¹¹;
R¹¹ and R¹³ are each independently H, —OH or C₁-C₆ alkyl;
R¹² is, at each occurrence, independently H, C₁-C₆ alkyl, aryl, cycloalkyl, or heterocyclyl;
n is, at each occurrence, independently 1 or 2;
m is, at each occurrence, independently 0, 1 or 2; and
x, y and z are each independently 0 or 1, wherein x, y and z are selected such that the sum of x+y+z is 1 or 2.

In one embodiment of Formula (VII), [symbol] is a single or double bond. In another embodiment of Formula (VII), [symbol] is a single bond. In another embodiment of Formula (VII), [symbol] is a double bond.

In one embodiment of Formula (VII), Q is —C(R¹⁰)ₙ—, —O—, —S(O)ₘ—, —NR⁹—, or —C(O)—. In another embodiment of Formula (VII), Q is —C(R¹⁰)ₙ—. In another embodiment of Formula (VII), Q is —O—. In another embodiment of Formula (VII), Q is —S(O)ₘ—. In another embodiment of Formula (VII), Q is —NR⁹—. In another embodiment of Formula (VII), Q is —C(O)—.

In one embodiment of Formula (VII), R¹ is carbocyclyl or heterocyclyl. In another embodiment of Formula (VII), R¹ is carbocyclyl. In another embodiment of Formula (VII), R¹ is heterocyclyl. In another embodiment of Formula (VII), R¹ is phenyl. In another embodiment of Formula (VII), R¹ is phenyl, which is unsubstituted. In another embodiment of Formula (VII), R¹ is phenyl, which is substituted. In another embodiment of Formula (VII), R¹ is tetrahydropyranyl, thiazolyl, oxazolyl, thiadiazolyl, pyrimidinyl, thiophenyl, furanyl, triazolyl, or pyridinyl.

In one embodiment of Formula (VII), R^{2a} and R^{2b} are each independently H, D, or C₁-C₆ alkyl. In another embodiment of Formula (VII), R^{2a} and R^{2b} are each independently H. In another embodiment of Formula (VII), R^{2a} is H and R^{2b} is C₁-C₆ alkyl.

In one embodiment of Formula (VII), R^{3a} is hydrogen, —C(O)NH₂, C₁-C₆ alkyl, aryl, cycloalkyl or heterocyclyl; and R^{3b} is C₁-C₆ alkyl, aryl, cycloalkyl or heterocyclyl; or R^{3a} and R^{3b} taken together with the carbon to which they are attached form aryl, cycloalkyl, or heterocycle. In another embodiment of Formula (VII), R^{3a} is C₁-C₆ alkyl; and R^{3b} is C₁-C₆ alkyl; wherein the C₁-C₆ alkyl is optionally substituted with one or more F, —CN, alkoxy, or SO₂R_{g}. In another embodiment of Formula (VII), R^{3a} is C₁-C₆ alkyl; and R^{3b} is CF₃. In another embodiment of Formula (VII), R^{3a} is CH₃; and R^{3b} is CF₃. In another embodiment of Formula (VII), R^{3a} is hydrogen; and R^{3b} is C₁-C₆ alkyl; wherein the C₁-C₆ alkyl is optionally substituted with one or more F, —CN, alkoxy, or SO₂R_{g}.

In another embodiment of Formula (VII), R^{3a} is hydrogen; and R^{3b} is cycloalkyl. In another embodiment of Formula (VII), R^{3a} is hydrogen; and R^{3b} is cyclopropyl. In another embodiment of Formula (VII), R^{3a} is C₁-C₆ alkyl; wherein the C₁-C₆ alkyl is optionally substituted with one or more F; and R^{3b} is cycloalkyl. In another embodiment of Formula (VII), R^{3a} is CH₃; and R^{3b} is cyclopropyl. In another embodiment of Formula (VII), R^{3a} is CF₃; and R^{3b} is cyclopropyl. In another embodiment of Formula (VII), R^{3a} is cycloalkyl; and R^{3b} is cycloalkyl. In another embodiment of Formula (VII), R^{3a} is cyclopropyl; and R^{3b} is cyclopropyl. In another embodiment of Formula (VII), R^{3a} is C₁-C₆ alkyl; and R^{3b} is heterocyclyl. In another embodiment of Formula (VII), R^{3a} is hydrogen; and R^{3b} is aryl. In another embodiment of Formula (VII), R^{3a} and R^{3b} taken together with the carbon to which they are attached form aryl, cycloalkyl, or heterocycle. In another embodiment of Formula (VII), R^{3a} and R^{3b} taken together with the carbon to which they are attached form aryl. In another embodiment of Formula (VII), R^{3a} and R^{3b} taken together with the carbon to which they are attached form cycloalkyl. In another embodiment of Formula (VII), R^{3a} and R^{3b} taken together with the carbon to which they are attached form heterocycle.

In one embodiment of Formula (VII), R^{5a} and R^{5b} are each independently H, C₁-C₆ alkyl or absent; or R^{5a} and R^{5b} taken together form oxo. In another embodiment of Formula (VII), R^{5a} and R^{5b} are each independently H, C₁-C₆ alkyl or absent. In another embodiment of Formula (VII), R^{5a} and R^{5b} taken together form oxo. In another embodiment of Formula (VII), R^{5a} and R^{5b} are each independently H. In another embodiment of Formula (VII), R^{5a} is H and R^{5b} is C₁-C₆ alkyl. In another embodiment of Formula (VII), R^{5a} and R^{5b} are absent.

In one embodiment of Formula (VII), R⁶ is independently H, halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (VII), R⁶ is H. In another embodiment of Formula (VII), R⁶ is —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (VII), R⁶ is heterocyclyl. In another embodiment of Formula (VII), R⁶ is pyrazolyl. In another embodiment of Formula (VII), R⁶ is —NHC(═O)NHR¹².

Still another embodiment pertains to compounds of Formula (VII), selected from the group consisting of: Examples 71-1, 88-1, 89-1, 96-1, 97-1, 100-1, 104-1, 108-1, 113-1, 117-1, 118-1, 121-1, 122-1, 123-1, 127-1, 128-1, 129-1, 130-1, 131-1, 132-1, 138-1, 139-1, 140-1, 143-1, 144-1, 147-1, 148-1, 151-1, 152-1, 153-1, 154-1, 157-1, 158-1, 159-1, 160-1, 161-1, 162-1, 163-1, 164-1, 165-1, 167-1, 169-1, 171-1, 172-1, 174-1, 175-1, 176-1, 177-1, 178-1, 179-1, 181-1, 184-1, 185-1, 186-1, 191-1, 192-1, 195-1, 198-1, 199-1, 200-1, 201-1, 202-1, 203-1, 204-1, 206-1, 208-1, 209-1, 210-1, 211-1, 212-1, 213-1, 214-1, 215-1, 216-1, 217-1, 218-1, 219-1, 220-1, 221-1, 222-1, 223-1, 224-1, 225-1, 226-1, 227-1, 228-1, 229-1, 230-1, 231-1, 232-1, 233-1, 234-1, 235-1, 235-2, 236-1, 237-1, 238-1, 242-1, 243-1, 245-1, 249-1, 250-1, 251-1, 258-1, 259-1, 260-1, 261-1, 261-2, 262-1, 263-1, 264-1, 265-1, 265-2, 266-1, 266-2, 267-1, 267-2, 268-1, 268-2, 268-3, 268-4, 269-1, 269-2, 270-1, 270-2, 271-1, 271-2, 272-1, 272-2, 273-1, 273-2, 274-1, 275-1, 275-2, 276-1, 277-1, 278-1, 279-1, 280-1, 281, 282, 283, 284, 285, 286, 287, 288, 291, 292, 293, 294, 297, 298, 299, 301, 302, 303, 304, 305, 306, 307, 308, 309, 310, 311, 312, 313, 314, 315, 316, 317, 318, 319, 320, 321, 324, 325, 326, 327, 328, 329, 330, 331, 332, 333, 334, 335, 336, 337, 338, 339, 340, 341, 342, 343, 344, 345, 349, 350, 352, 353, 354, 359, 361, 362, 363, 364, 365, 367, 368, 375, 377, 378, 382, 383, 385, 387, 388, 389, 392, 393, 394, 395, 396, 397, 398, 399, 400, 401, 402, 403, 404, 405, 406, 407, 408, 409, 411, 412, 413, 414, 415, 416, 417, 418, 419, 420, 421, 422, 424, 426, 427, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 490, 494, 496, 497, 498, 502, 503, 504, 506, 507, 508, 509, 511, 512, 516, 517, 519, 520, 521, 523, 524, 525, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 565, 566, 567, 568, 572, 573, 574, 575, 576, 577, 578, 581, 583, 584, 585, 586, 587, 588, 589, 590, 591, 592, 593, 594, 595, 596, 599, 600, 601, 602, 603, 604, 606, 607, 608, 609, 610, 611, 612, 613, 614, 615, 616, 622, 624, 626, 627, 628, 631, 632, 633, 634, 635, 636, 639, 642, 648, 649, 651, 652, 653, 654, 656, 658, 659, 660, 661, 662, 663, 664, 665, 666, 668, 669, 670, 671, 672, 674, 675, 676, 677, 678, 684, 687, 688, 689, 694, 695, 696, 697, 702, 707, 708, 709, 710, 712, 713, 714, 718, 719, 720, 721, 722, 723, 725, 726, 727, 728, 729, 730, 732, 733, 734, 736, 737, 752, 754, 755, 756, 757, 758, and pharmaceutically acceptable salts thereof.

In another aspect, the present invention relates to compounds of Formula (VIII) or a pharmaceutically acceptable salt thereof,

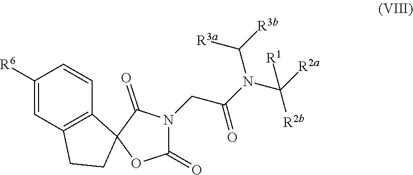

or a stereoisomer, tautomer or pharmaceutically acceptable salt thereof,
wherein:
R¹ is carbocyclyl or heterocyclyl;
R^{2a} and R^{2b} are each independently H, D, or C₁-C₆ alkyl;
R^{3a} is hydrogen, C(O)NH₂, C₁-C₆ alkyl, aryl, cycloalkyl or heterocyclyl; and
R^{3b} is C₁-C₆ alkyl, aryl, cycloalkyl or heterocyclyl; or
R^{3a} and R^{3b} taken together with the carbon to which they are attached form aryl, cycloalkyl, or heterocycle;
R⁶ is independently halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹²;
R¹¹ and R¹³ are each independently H, —OH or C₁-C₆ alkyl; and
R¹² is, at each occurrence, independently H, C₁-C₆ alkyl, aryl, cycloalkyl, or heterocyclyl.

In one embodiment of Formula (VIII), R¹ is carbocyclyl or heterocyclyl. In another embodiment of Formula (VIII), R¹ is carbocyclyl. In another embodiment of Formula (VIII), R¹ is heterocyclyl. In another embodiment of Formula (VIII), R¹ is phenyl. In another embodiment of Formula (VIII), R¹ is phenyl, which is unsubstituted. In another embodiment of Formula (VIII), R¹ is phenyl, which is substituted. In another embodiment of Formula (VIII), R¹ is tetrahydropyranyl, thiazolyl, oxazolyl, thiadiazolyl, pyrimidinyl, thiophenyl, furanyl, triazolyl, or pyridinyl.

In one embodiment of Formula (VIII), R^{2a} and R^{2b} are each independently H, D, or C₁-C₆ alkyl. In another embodiment of Formula (VIII), R^{2a} and R^{2b} are each independently H. In another embodiment of Formula (VIII), R^{2a} is H and R^{2b} is C₁-C₆ alkyl.

In one embodiment of Formula (VIII), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; or R^{3a} and R^{3b} taken together with the carbon to which they are attached form aryl, cycloalkyl, or heterocycle. In another embodiment of Formula (VIII), R^{3a} is C₁-C₆ alkyl; and R^{3b} is C₁-C₆ alkyl; wherein the C₁-C₆ alkyl is optionally substituted with one or more F, —CN, alkoxy, or SO₂R_{g}. In another embodiment of Formula (VIII), R^{3a} is C₁-C₆ alkyl; and R^{3b} is CF₃. In another embodiment of Formula (VIII), R^{3a} is CH₃; and R^{3b} is CF₃. In another embodiment of Formula (VIII), R^{3a} is hydrogen; and R^{3b} is C₁-C₆ alkyl; wherein the C₁-C₆ alkyl is optionally substituted with one or more F, —CN, alkoxy, or SO₂R_{g}. In another embodiment of Formula (VIII), R^{3a} is hydrogen; and R^{3b} is cycloalkyl. In another embodiment of Formula (VIII), R^{3a} is hydrogen; and R^{3b} is cyclopropyl. In another embodiment of Formula (VIII), R^{3a} is C₁-C₆ alkyl; wherein the C₁-C₆ alkyl is optionally substituted with one or more F; and R^{3b} is cycloalkyl. In another embodiment of Formula (VIII), R^{3a} is CH₃; and R^{3b} is cyclopropyl. In another embodiment of Formula (VIII), R^{3a} is CF₃; and R^{3b} is cyclopropyl. In another embodiment of Formula (VIII), R^{3a} is cycloalkyl; and R^{3b} is cycloalkyl. In another embodiment of Formula (VIII), R^{3a} is cyclopropyl; and R^{3b} is cyclopropyl. In another embodiment of Formula (VIII), R^{3a} is C₁-C₆ alkyl; and R^{3b} is heterocyclyl. In another embodiment of Formula (VIII), R^{3a} is hydrogen; and R^{3b} is aryl. In another embodiment of Formula (VIII), R^{3a} and R^{3b} taken together with the carbon to which they are attached form aryl, cycloalkyl, or heterocycle. In another embodiment of Formula (VIII), R^{3a} and R^{3b} taken together with the carbon to which they are attached form aryl. In another embodiment of Formula (VIII), R^{3a} and R^{3b} taken together with the carbon to which they are attached form cycloalkyl. In another embodiment of Formula (VIII), R^{3a} and R^{3b} taken together with the carbon to which they are attached form heterocycle.

In one embodiment of Formula (VIII), R⁶ is independently H, halo, —OH, —CN, —CO₂H, C₁-C₆ alkyl, C₂-C₆ alkynyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (VIII), R⁶ is H. In another embodiment of Formula (VIII), R⁶ is —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹², —NHC(═O)NR¹²S(O)₂OR¹², —NHC(═O)NR¹²S(O)₂NHR¹², —NHC(═O)N(R¹²)₂, —NHC(═S)NHR¹², —NHC(═N—C≡N)NR¹², —NHC(═N—C≡N)SR¹², or —NHS(O)ₘR¹². In another embodiment of Formula (VIII), R⁶ is heterocyclyl. In another embodiment of Formula (VIII), R⁶ is pyrazolyl. In another embodiment of Formula (VIII), R⁶ is —NHC(═O)NHR¹².

Still another embodiment pertains to compounds of Formula (VIII), selected from the group consisting of: Examples 71-1, 88-1, 89-1, 96-1, 97-1, 100-1, 104-1, 108-1, 113-1, 117-1, 118-1, 121-1, 122-1, 123-1, 127-1, 128-1, 129-1, 130-1, 131-1, 132-1, 138-1, 139-1, 140-1, 143-1, 144-1, 147-1, 148-1, 151-1, 152-1, 153-1, 154-1, 157-1, 158-1, 159-1, 160-1, 161-1, 162-1, 163-1, 164-1, 165-1, 167-1, 169-1, 171-1, 172-1, 174-1, 175-1, 176-1, 177-1, 178-1, 179-1, 181-1, 184-1, 185-1, 186-1, 191-1, 192-1, 195-1, 198-1, 199-1, 200-1, 201-1, 202-1, 203-1, 204-1, 206-1, 208-1, 209-1, 210-1, 211-1, 212-1, 213-1, 214-1, 215-1, 216-1, 217-1, 218-1, 219-1, 220-1, 221-1, 222-1, 223-1, 224-1, 225-1, 226-1, 227-1, 228-1, 229-1, 230-1, 231-1, 232-1, 233-1, 234-1, 235-1, 235-2, 236-1, 237-1, 238-1, 242-1, 243-1, 245-1, 249-1, 250-1, 251-1, 258-1, 259-1, 260-1, 261-1, 261-2, 262-1, 263-1, 264-1, 265-1, 265-2, 266-1, 266-2, 267-1, 267-2, 268-1, 268-2, 268-3, 268-4, 269-1, 269-2, 270-1, 270-2, 271-1, 271-2, 272-1, 272-2, 273-1, 273-2, 274-1, 275-1, 275-2, 276-1, 277-1, 278-1, 279-1, 280-1, 281, 282, 283, 284, 285, 286, 287, 288, 291, 292, 293, 294, 297, 298, 299, 302, 303, 304, 305, 306, 307, 308, 309, 310, 311, 312, 313, 314, 315, 316, 317, 318, 319, 320, 324, 325, 326, 327, 328, 329, 330, 331, 332, 333, 334, 335, 336, 337, 338, 339, 340, 341, 342, 343, 344, 345, 352, 353, 354, 361, 362, 363, 364, 365, 375, 377, 378, 383, 387, 388, 389, 392, 393, 394, 395, 396, 397, 398, 399, 400, 401, 402, 403, 404, 405, 406, 407, 408, 409, 411, 412, 413, 414, 415, 416, 417, 418, 419, 420, 421, 422, 426, 427, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 490, 496, 497, 498, 502, 503, 504, 506, 507, 508, 509, 511, 512, 516, 517, 519, 520, 521, 523, 524, 525, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 565, 566, 567, 568, 572, 573, 574, 575, 576, 577, 578, 583, 587, 589, 590, 591, 592, 593, 594, 595, 599, 600, 602, 603, 606, 607, 608, 609, 610, 611, 612, 613, 614, 615, 616, 622, 624, 626, 627, 628, 631, 632, 633, 634, 635, 636, 639, 642, 648, 649, 651, 652, 653, 654, 656, 658, 659, 660, 661, 662, 663, 664, 665, 666, 668, 669, 670, 671, 672, 674, 675, 676, 677, 678, 684, 687, 688, 689, 694, 695, 696, 697, 702, 707, 708, 709, 710, 712, 713, 714, 718, 719, 720, 721, 722, 723, 725, 726, 727, 728, 729, 730, 732, 733, 734, 736, 737, 752, 754, 755, 756, 757, 758, and pharmaceutically acceptable salts thereof.

### Compounds of Formula (IX), (Xa), (Xb), (XIa), (XIb), (XIIa), (XIIb), (XIIIa), (XIIIb), (XIVa), (XIVb), (XVa), (XVb), (XVIa), and (XVIb)

Suitable groups for A B, [symbol] , W, R¹, R^{2a}, R^{2b}, R^{3a}, R^{3b}, R^{4a}, R^{4b}, R⁶, R⁷, x, and y in compounds of Formula (IX); [symbol] , W, R¹, R^{2a}, R^{2b}, R^{3a}, R^{3b}, R^{4a}, R^{4b}, R⁶, R⁷, x, and y in compounds of Formula (Xa) and Formula (Xb); [symbol] , R¹, R^{2a}, R^{2b}, R^{3a}, R^{3b}, R⁶, R⁷, x, and y in compounds of Formula (XIa) and Formula (XIb); [symbol] , R¹, R^{2a}, R^{2b}, R^{3a}, R^{3b}, R⁶, x, and y in compounds of Formula (XIIa) and Formula (XIIb); and R¹, R^{2a}, R^{2b}, R^{3a}, R^{3b}, and R⁶ in compounds of Formula (XIIIa), Formula (XIIIb), Formula (XIVa), Formula (XIVb), Formula (XVa), and Formula (XVb); are independently selected. The described embodiments of the present invention may be combined. Such combination is contemplated and within the scope of the present invention. For example, it is contemplated that embodiments for any of A, B, [symbol] , W, R¹, R^{2a}, R^{2b}, R^{3a}, R^{3b}, R^{4a}, R^{4b}, R⁶, R⁷, x, and y, in compounds of Formula (IX) can be combined with embodiments defined for any other of A, B, [symbol] , W, R¹, R^{2a}, R^{2b}, R^{3a}, R^{3b}, R^{4a}, R^{4b}, R⁶, R⁷, x, and y in compounds of Formula (IX).

In one aspect, the present invention relates to compounds of Formula (IX),

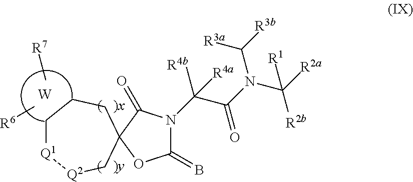

or a stereoisomer, tautomer or pharmaceutically acceptable salt thereof,
wherein
[symbol] is —C(R¹⁰)₂—C(R¹⁴)₂—, —O—C(R¹⁴)₂—, —O—C(O)—, —S(O)₂—C(R¹⁴)₂—, —S—C(R¹⁴)₂—, —NR⁹—C(O)—, —NR⁹—C(R¹⁴)₂—, —C(R¹⁰)₂—O—, —C(R¹⁰)₂—, or —C(R¹⁰)═C(R¹⁴)—;
A is —NR⁸—, —O—, or —S—;
B is O or NH;
W is arylene or heteroarylene;
R¹ is carbocyclyl or heterocyclyl;
R^{2a} and R^{2b} are each independently H, D, or C₁-C₆ alkyl;
R^{3a} is hydrogen, C(O)NH₂, C₁-C₆ alkyl, aryl, cycloalkyl or heterocyclyl; and
R^{3b} is C₁-C₆ alkyl, aryl, cycloalkyl or heterocyclyl; or
R^{3a} and R^{3b} taken together with the carbon to which they are attached form an arene, cycloalkane, or heterocycle;
R^{4a} and R^{4b} are each independently H, D or C₁-C₆ alkyl;
R⁶ and R⁷ are each independently H, halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═S)N(R¹²)₂, —NHC(═N—C≡N)NR¹², —NHC(═N—C≡N)SR¹², or —NHS(O)ₘR¹²;
R⁸ and R⁹ are each independently H or C₁-C₆ alkyl;
R¹⁰, at each occurrence, is independently H, —OH, halo, —CN, —CO₂R¹², —C(═O)NHR¹³, —NHR¹², C₁-C₆ alkyl or alkoxy; or two R¹⁰ taken together form oxo or ═N—OR¹¹;
R¹¹ and R¹³ are each independently H, —OH, or C₁-C₆ alkyl;
R¹², at each occurrence, is independently H, C₁-C₆ alkyl, aryl, cycloalkyl, or heterocyclyl;
R¹⁴, at each occurrence, is independently H or C₁-C₆ alkyl;
m, at each occurrence, is independently 0, 1 or 2; and
x, and y are each independently 0 or 1, wherein x and y are selected such that the sum of x+y is 0 or 1;

with the proviso that R^{3a} and R^{3b} are not cyclopropyl and methyl, respectively when R¹ and W are each unsubstituted phenyl, A is —NH, x is 0 or 1, y is 0, and [symbol] is —C(R¹⁰)₂—C(R¹⁴)₂—; and

with the proviso that R^{3a} and R^{3b} taken together with the carbon to which they are attached do not form tetrahydrothiophene 1,1-dioxide or tetrahydrothiophene when at least one of R¹ and W is unsubstituted phenyl, and A is —NH.

In one embodiment of Formula (IX), A is —NR⁸—, —O—, or —S—. In another embodiment of Formula (IX), A is —NR⁸—, or —O—. In another embodiment of Formula (IX), A is —NR⁸—. In another embodiment of Formula (IX), A is —O—.

In one embodiment of Formula (IX), B is O or NH. In another embodiment of Formula (IX), B is O. In another embodiment of Formula (IX), B is NH.

In one embodiment of Formula (IX), [symbol] is —C(R¹⁰)₂—C(R¹⁴)₂—, —O—C(R¹⁴)₂—, —O—C(O)—, —S(O)₂—C(R¹⁴)₂—, —S—C(R¹⁴)₂—, —NR⁹—C(O)—, —NR⁹—C(R¹⁴)₂—, —C(R¹⁰)₂—O—, —C(R¹⁰)₂—, or —C(R¹⁰)═C(R¹⁴)—. In another embodiment of Formula (IX), [symbol] is C(R¹⁰)₂—C(R¹⁴)₂—. In another embodiment of Formula (IX), [symbol] is —O—C(R¹⁴)₂—. In another embodiment of Formula (IX), [symbol] is —O—C(O)—. In another embodiment of Formula (IX), [symbol] is —S(O)₂—C(R¹⁴)₂—. In another embodiment of Formula (IX), [symbol] is —S—C(R¹⁴)₂—. In another embodiment of Formula (IX), [symbol] is —NR⁹—C(O)—. In another embodiment of Formula (IX), [symbol] is —NR⁹—C(R¹⁴)₂—. In another embodiment of Formula (IX), [symbol] is —C(R¹⁰)₂—O—. In another embodiment of Formula (IX), [symbol] is —C(R¹⁰)₂—. In another embodiment of Formula (IX), [symbol] is —C(R¹⁰)═C(R¹⁴)—.

In one embodiment of Formula (IX), W is arylene or heteroarylene. In another embodiment of Formula (IX), W is arylene. In another embodiment of Formula (IX), W is heteroarylene. In another embodiment of Formula (IX), W is

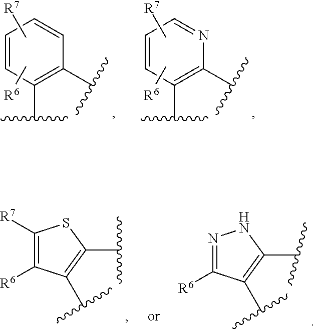

In another embodiment of Formula (IX), W is

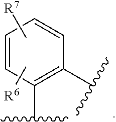

In another embodiment of Formula (IX), W is

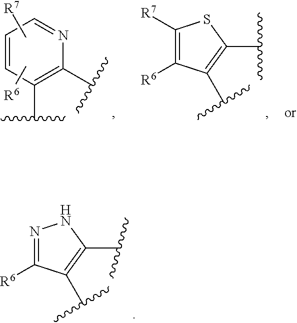

In one embodiment of Formula (IX), R¹ is carbocyclyl or heterocyclyl. In another embodiment of Formula (IX), R¹ is carbocyclyl. In another embodiment of Formula (IX), R¹ is heterocyclyl. In another embodiment of Formula (IX), R¹ is phenyl, naphthyl, cyclopropyl, or cyclobutyl. In another embodiment of Formula (IX), R¹ is phenyl. In another embodiment of Formula (IX), R¹ is phenyl, which is unsubstituted. In another embodiment of Formula (IX), R¹ is phenyl, which is substituted. In another embodiment of Formula (IX), R¹ is phenyl, which is substituted with F, Cl, Br, CN, —NR_{g}Rₕ, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, aryl, and/or haloalkyl; wherein alkyl, alone or part of a group, is optionally substituted with F, C(═O)NR_{g}Rₕ, or CN. In another embodiment of Formula (IX), R¹ is phenyl, which is substituted with F, Cl, Br, CN, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, and/or haloalkyl; wherein alkyl, alone or part of a group, is optionally substituted with F. In another embodiment of Formula (IX), R¹ is phenyl, which is substituted with F. In another embodiment of Formula (IX), R¹ is tetrahydropyranyl, thiazolyl, oxazolyl, thiadiazolyl, pyrimidinyl, thiophenyl, furanyl, triazolyl, indolyl, imidazolyl, or pyridinyl. In another embodiment of Formula (IX), R¹ is tetrahydropyranyl, thiazolyl, oxazolyl, thiadiazolyl, pyrimidinyl, thiophenyl, furanyl, triazolyl, indolyl, imidazolyl, or pyridinyl; which is unsubstituted. In another embodiment of Formula (IX), R¹ is tetrahydropyranyl, thiazolyl, oxazolyl, thiadiazolyl, pyrimidinyl, thiophenyl, furanyl, triazolyl, indolyl, imidazolyl, or pyridinyl; which is substituted. In another embodiment of Formula (IX), R¹ is tetrahydropyranyl, thiazolyl, oxazolyl, thiadiazolyl, pyrimidinyl, thiophenyl, furanyl, triazolyl, indolyl, imidazolyl, or pyridinyl; which is substituted with F, Cl, Br, CN, —NR_{g}Rₕ, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, aryl, and/or haloalkyl; wherein alkyl, alone or part of a group, is optionally substituted with F, C(═O)NR_{g}Rₕ, or CN.

In one embodiment of Formula (IX), R^{2a} and R^{2b} are each independently H, D, or C₁-C₆ alkyl. In another embodiment of Formula (IX), R^{2a} and R^{2b} are each independently H. In another embodiment of Formula (IX), R^{2a} is H and R^{2b} is C₁-C₆ alkyl. In another embodiment of Formula (IX), R^{2a} is H and R^{2b} is C₁-alkyl.

In one embodiment of Formula (IX), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; or R^{3a} and R^{3b} taken together with the carbon to which they are attached form arene, cycloalkane, or heterocycle. In another embodiment of Formula (IX), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; wherein C₁-C₆ alkyl, is optionally substituted with F, —CN, —NR_{g}Rₕ, —OR_{g}, or —SO₂R_{g}; wherein R_{g} and Rₕ are the same or different and independently hydrogen or alkyl; wherein aryl, cycloalkyl and heterocyclyl are optionally substituted with alkyl, —SO₂NR_{g}C(═O)ORₕ, —SO₂NR_{g}Rₕ, —C(═O)R_{g}, —C(═O)OR_{g}, or —C(═O)NR_{g}Rₕ; wherein R_{g} and Rₕ are the same or different and independently hydrogen or alkyl; wherein alkyl, alone or part of a group, is optionally substituted with aryl or —C(═O)OH.

In another embodiment of Formula (IX), R^{3a} is C₁-C₆ alkyl; and R^{3b} is C₁-C₆ alkyl; wherein the C₁-C₆ alkyl is optionally substituted with one or more F, —CN, alkoxy, or —SO₂R_{g}. In another embodiment of Formula (IX), R^{3a} is C₁-C₆ alkyl; and R^{3b} is CF₃. In another embodiment of Formula (IX), R^{3a} is CH₃; and R^{3b} is CF₃. In another embodiment of Formula (IX), R^{3a} is hydrogen; and R^{3b} is C₁-C₆ alkyl; wherein the C₁-C₆ alkyl is optionally substituted with one or more F, —CN, alkoxy, or —SO₂R_{g}. In another embodiment of Formula (IX), R^{3a} is hydrogen; and R^{3b} is cycloalkyl. In another embodiment of Formula (IX), R^{3a} is hydrogen; and R^{3b} is cyclopropyl. In another embodiment of Formula (IX), R^{3a} is C₁-C₆ alkyl; wherein the C₁-C₆ alkyl is CH₃; and R^{3b} is cycloalkyl. In another embodiment of Formula (IX), R^{3a} is CH₃; and R^{3b} is cyclopropyl. In another embodiment of Formula (IX), R^{3a} is CF₃; and R^{3b} is cyclopropyl. In another embodiment of Formula (IX), R^{3a} is cycloalkyl; and R^{3b} is cycloalkyl. In another embodiment of Formula (IX), R^{3a} is cyclopropyl; and R^{3b} is cyclopropyl. In another embodiment of Formula (IX), R^{3a} is C₁-C₆ alkyl; and R^{3b} is heterocyclyl. In another embodiment of Formula (IX), R^{3a} is hydrogen; and R^{3b} is aryl. In another embodiment of Formula (IX), R^{3a} and R^{3b} taken together with the carbon to which they are attached form arene, cycloalkane, or heterocycle. In another embodiment of Formula (IX), R^{3a} and R^{3b} taken together with the carbon to which they are attached form arene. In another embodiment of Formula (IX), R^{3a} and R^{3b} taken together with the carbon to which they are attached form cycloalkane. In another embodiment of Formula (IX), R^{3a} and R^{3b} taken together with the carbon to which they are attached form heterocycle. In another embodiment of Formula (IX), R^{3a} and R^{3b} taken together with the carbon to which they are attached form cyclopropane, azetidine, cyclobutane, tetrahydrofuran, pyrrolidine, cyclopentane, cyclohexane, 2-azaspiro[3.3]heptane, tetrahydro-2H-thiopyran 1, 1 dioxide, piperidine, or benzene. In another embodiment of Formula (IX), R^{3a} and R^{3b} taken together with the carbon to which they are attached form cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, or benzene wherein cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, or benzene are optionally substituted with alkyl, haloalkyl, —F, —CN, —NR_{g}Rₕ, —NR_{g}C(═O)Rₕ, —NR_{g}SO₂Rₕ, —OR_{g}, —SO₂R_{g}, —NR_{g}SO₂NR_{g}C(═O)Rₕ, —SO₂NR_{g}C(═O)ORₕ, —SO₂NR_{g}Rₕ, —C(═O)R_{g}, —C(═O)OR_{g}, or —C(═O)NR_{g}Rₕ; wherein R_{g} and Rₕ are the same or different and independently hydrogen, alkyl, or haloalkyl; wherein each alkyl, alone or part of a group, is optionally substituted with CN, alkyl, —SO₂NH₂, C(═O)NHCH₃, —C(═O)OC(CH₃)₃, —C(═O)OCH₃, or —C(═O)OH.

In one embodiment of Formula (IX), R^{4a} and R^{4b} are each independently H, D or C₁-C₆ alkyl. In another embodiment of Formula (IX), R^{4a} is H and R^{4b} is C₁-C₆ alkyl. In another embodiment of Formula (IX), R^{4a} and R^{4b} are each independently H.

In one embodiment of Formula (IX), R⁶ and R⁷ are each independently H, halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═S)N(R¹²)₂, —NHC(═N—C≡N)NR¹², —NHC(═N—C≡N)SR¹², or —NHS(O)ₘR¹². In another embodiment of Formula (IX), R⁷ is H and R⁶ is H, halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═S)N(R¹²)₂, —NHC(═N—C≡N)NR¹², —NHC(═N—C≡N)SR¹², or —NHS(O)ₘR¹². In another embodiment of Formula (IX), R⁷ is H and R⁶ is —N(R¹²)₂, —C(═O)NHR¹², —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═O)N(R¹²)₂, —NHC(═S)N(R¹²)₂, —NHC(═N—C≡N)NR¹², —NHC(═N—C≡N)SR¹², or —NHS(O)ₘR¹². In another embodiment of Formula (IX), R⁷ is H and R⁶ is heterocyclyl. In another embodiment of Formula (IX), R⁷ is H and R⁶ is pyrazolyl. In another embodiment of Formula (IX), R⁷ is H and R⁶ is —NHC(═O)NHR¹².

In one embodiment of Formula (IX), R⁸ and R⁹ are each independently H or C₁-C₆ alkyl. In another embodiment of Formula (IX), R⁸ and R⁹ are each independently H. In another embodiment of Formula (IX), R⁸ and R⁹ are each independently C₁-C₆ alkyl.

In one embodiment of Formula (IX), R¹⁰, at each occurrence, is independently H, —OH, halo, —CN, —CO₂R¹², —C(═O)NHR¹³, —NHR¹², C₁-C₆ alkyl or alkoxy; or two R¹⁰ taken together form oxo or ═N—OR¹¹. In another embodiment of Formula (IX), R¹⁰, at each occurrence, is independently H, —OH, halo, —CN, —CO₂R¹², —C(═O)NHR¹³, —NHR¹², C₁-C₆ alkyl or alkoxy; or two R¹⁰ taken together form oxo or ═N—OR¹¹. In another embodiment of Formula (IX), R¹⁰, at each occurrence, is independently H. In another embodiment of Formula (IX), R¹⁰, at each occurrence, is independently H or —OH. In another embodiment of Formula (IX), R¹⁰, at each occurrence, is independently H or F. In another embodiment of Formula (IX), one R¹⁰ is independently H, and the remaining is F. In another embodiment of Formula (IX), R¹⁰, at each occurrence, is independently H or halo. In another embodiment of Formula (IX), R¹⁰, at each occurrence, is independently H or —CN. In another embodiment of Formula (IX), R¹⁰, at each occurrence, is independently H or —CO₂R¹². In another embodiment of Formula (IX), R¹⁰, at each occurrence, is independently H or —C(═O)NHR¹³. In another embodiment of Formula (IX), R¹⁰, at each occurrence, is independently H or —NHR¹². In another embodiment of Formula (IX), R¹⁰, at each occurrence, is independently H or C₁-C₆ alkyl. In another embodiment of Formula (IX), R¹⁰, at each occurrence, is independently H or alkoxy. In another embodiment of Formula (IX), two R¹⁰ taken together form oxo. In another embodiment of Formula (IX), two R¹⁰ taken together form ═N—OR¹¹.

In one embodiment of Formula (IX), R¹¹ and R¹³ are each independently H, —OH, or C₁-C₆ alkyl. In another embodiment of Formula (IX), R¹¹ and R¹³ are each independently H. In another embodiment of Formula (IX), R¹¹ and R¹³ are each independently —OH. In another embodiment of Formula (IX), R¹¹ and R¹³ are each independently C₁-C₆ alkyl.

In one embodiment of Formula (IX), R¹², at each occurrence, is independently H, C₁-C₆ alkyl, aryl, cycloalkyl, or heterocyclyl. In another embodiment of Formula (IX), R¹², at each occurrence, is independently H, C₁-C₆ alkyl, aryl, cycloalkyl, or heterocyclyl; wherein C₁-C₆ alkyl, aryl, cycloalkyl, and heterocyclyl are optionally substituted with F, alkyl, alkoxy, cycloalkyl, haloalkyl, heterocyclyl, heterocyclylalkyl, oxo, CN, —NR_{g}Rₕ, —NR_{g}C(═O)Rₕ, —NR_{g}C(═O)ORₕ, —OR_{g}, —SO₂R_{g}, —SO₂NR_{g}Rₕ, —C(═O)R_{g}, —C(═O)OR_{g}, or —C(═O)NR_{g}Rₕ; wherein R_{g} and Rₕ are the same or different and independently hydrogen, alkyl, alkoxy, aryl, cycloalkyl, and/or haloalkyl.

In one embodiment of Formula (IX), R¹⁴, at each occurrence, is independently H or C₁-C₆ alkyl. In another embodiment of Formula (IX), R¹⁴, at each occurrence, is independently H. In another embodiment of Formula (IX), R¹⁴, at each occurrence, is independently C₁-C₆ alkyl.

In one embodiment of Formula (IX), m, at each occurrence, is independently 0, 1 or 2. In another embodiment of Formula (IX), m, at each occurrence, is independently 0. In another embodiment of Formula (IX), m, at each occurrence, is independently 1. In another embodiment of Formula (IX), m, at each occurrence, is independently 2.

In one embodiment of Formula (IX), x, and y are each independently 0 or 1, wherein x and y are selected such that the sum of x+y is 0 or 1. In another embodiment of Formula (IX), x is 0 and y is 0. In another embodiment of Formula (IX), x is 0 and y is 1. In another embodiment of Formula (IX), x is 1 and y is 0.

Still another embodiment pertains to compounds of Formula (IX), selected from the group consisting of:
N-(4-bromobenzyl)-N-(1-cyclopropylethyl)-2-(2,5-dioxo-2′,3′-dihydrospiro[imidazolidine-4,1′-indene]-1-yl)acetamide;
N-(1-cyclopropylethyl)-2-(2,5-dioxo-2′,3′-dihydrospiro[imidazolidine-4,1′-indene]-1-yl)-N-(furan-2-ylmethyl)acetamide;
N-benzyl-N-(1-cyclopropylethyl)-2-(1′-methyl-2,2′,5-trioxospiro[imidazolidine-4,3′-indoline]-1-yl)acetamide;
N-(1-cyclopropylethyl)-2-(2,5-dioxo-2′,3′-dihydrospiro[imidazolidine-4,1′-indene]-1-yl)-N-(thiophen-2-ylmethyl)acetamide;
N-benzyl-N-(1-cyclopropylethyl)-2-(3-methyl-2,5-dioxo-2′,3′-dihydrospiro[imidazolidine-4,1′-indene]-1-yl)acetamide;
N-(1-cyclopropylethyl)-2-(2,5-dioxo-2′,3′-dihydrospiro[imidazolidine-4,1′-indene]-1-yl)-N-(4-fluorobenzyl)acetamide;
N-(1-cyclopropylethyl)-2-(2,5-dioxo-2′,3′-dihydrospiro[imidazolidine-4,1′-indene]-1-yl)-N-(2-fluorobenzyl)acetamide;
N-(1-cyclopropylethyl)-2-(2,5-dioxo-2′,3′-dihydrospiro[imidazolidine-4,1′-indene]-1-yl)-N-(3-fluorobenzyl)acetamide;
N-benzyl-2-(5′-bromo-2,5-dioxo-2′,3′-dihydrospiro[imidazolidine-4,1′-indene]-1-yl)-cyclopropylethyl)acetamide;
2-(5′-bromo-2,5-dioxo-2′,3′-dihydrospiro[imidazolidine-4,1′-indene]-1-yl)-N-(1-cyclopropylethyl)-N-(furan-2-ylmethyl)acetamide;
N-benzyl-N-(1-cyclopropylethyl)-2-(5′-fluoro-2,5-dioxo-2′,3′-dihydrospiro[imidazolidine-4,1′-indene]-1-yl)acetamide;
N-benzyl-2-(4′-bromo-2,5-dioxo-2′,3′-dihydrospiro[imidazolidine-4,1′-indene]-1-yl)-N-(1-cyclopropylethyl)acetamide;
N-(1-cyclopropylethyl)-2-(2,5-dioxo-2′,3′-dihydrospiro[imidazolidine-4,1′-indene]-1-yl)-N-(pyridin-3-ylmethyl)acetamide;
N-(1-cyclopropylethyl)-2-(2,5-dioxo-2′,3′-dihydrospiro[imidazolidine-4,1′-indene]-1-yl)-N-(pyridin-2-ylmethyl)acetamide;
N-benzyl-N-(1-cyclopropylethyl)-2-(5′-methoxy-2,5-dioxo-2′,3′-dihydrospiro[imidazolidine-4,1′-indene]-1-yl)acetamide;
N-benzyl-N-(1-cyclopropylethyl)-2-(4′-methoxy-2,5-dioxo-2′,3′-dihydrospiro[imidazolidine-4,1′-indene]-1-yl)acetamide;
N-benzyl-N-(1-cyclopropylethyl)-2-(2′,5′-dioxo-6,7-dihydrospiro[cyclopenta[b]pyridine-5,4′-imidazolidine]-1′-yl)acetamide;
N-benzyl-2-(5′-cyano-2,5-dioxo-2′,3′-dihydrospiro[imidazolidine-4,1′-indene]-1-yl)-N-(1-cyclopropylethyl)acetamide;
N-(1-cyclopropylethyl)-2-(2,5-dioxo-2′,3′-dihydrospiro[imidazolidine-4,1′-indene]-1-yl)-N-(pyridin-4-ylmethyl)acetamide;
N-benzyl-2-(4′-chloro-2,5-dioxo-2′,3′-dihydrospiro[imidazolidine-4,1′-indene]-1-yl)-N-(1-cyclopropylethyl)acetamide;
N-benzyl-2-(2,5-dioxo-2′,3′-dihydrospiro[imidazolidine-4,1′-indene]-1-yl)-N-isopropylacetamide;
N-benzyl-N-sec-butyl-2-(2,5-dioxo-2′,3′-dihydrospiro[imidazolidine-4,1′-indene]-1-yl)acetamide;
N-(1-cyclobutylethyl)-2-(2,5-dioxo-2′,3′-dihydrospiro[imidazolidine-4,1′-indene]-1-yl)-N-(4-fluorobenzyl)acetamide;
N-benzyl-2-(4′-bromo-2,5-dioxo-2′,3′-dihydrospiro[imidazolidine-4,1′-indene]-1-yl)-N—((R)-1-cyclobutylethyl)acetamide;
2-(4′-bronco-2,5-dioxo-2′,3′-dihydrospiro[imidazolidine-4,1′-indene]-1-yl)-N-(1-cyclopropylethyl)-N-(4-fluorobenzyl)acetamide,
N-benzyl-2-(5′-bromo-2,5-dioxo-2′,3′-dihydrospiro[imidazolidine-4,1′-indene]-1-yl)-N-(1-cyclobutylethyl)acetamide;
2-(5′-bromo-2,5-dioxo-2′,3′-dihydrospiro[imidazolidine-4,1′-indene]-1-yl)-N-(1-cyclopropylethyl)-N-(4-fluorobenzyl)acetamide;
1-(2-(benzyl(1-cyclopropylethyl)amino)-2-oxoethyl)-2,5-dioxo-2′,3′-dihydrospiro[imidazolidine-4,1′-indene]-5′-carboxamide;
N-benzyl-2-(4′-cyano-2,5-dioxo-2′,3′-dihydrospiro[imidazolidine-4,1′-indene]-1-yl)-N-(1-cyclopropylethyl)acetamide;
1-(2-(benzyl(1-cyclopropylethyl)amino)-2-oxoethyl)-2,5-dioxo-2′,3′-dihydrospiro[imidazolidine-4,1′-indene]-4′-carboxamide;
N-(1-cyclopropylethyl)-2-(2,5-dioxo-2′,3′-dihydrospiro[imidazolidine-4,1′-indene]-1-yl)-N-(naphthalen-2-ylmethyl)acetamide,
N-((5-bromofuran-2-yl)methyl)-N-(1-cyclopropylethyl)-2-(2,5-dioxo-2′,3′-dihydrospiro[imidazolidine-4,1′-indene]-1-yl)acetamide;
N-benzyl-N—((S)-1-cyclobutylethyl)-2-((S)-2,5-dioxo-2′,3′-dihydrospiro[imidazolidine-4,1′-indene]-1-yl)acetamide;
N-benzyl-N—((R)-1-cyclobutylethyl)-2-((S)-5-dioxo-2′,3′-dihydrospiro[imidazolidine-4,1′-indene]-1-yl)acetamide;
N-benzyl-N-(1-cyclopropylethyl)-2-(2′,5′-dioxo-5,6-dihydrospiro[cyclopenta[b]thiophene-4,1′-imidazolidine]-1′-yl)acetamide;
N-benzyl-N-(1-cyclopropylethyl)-2-(2′,5′-dioxo-4,5-dihydrospiro[cyclopenta[b]thiophene-6,4′-imidazolidine]-1′-yl)acetamide;
N-((1H-indol-5-yl)methyl)-N-(1-cyclopropylethyl)-2-(2,5-dioxo-2′,3′-dihydrospiro[imidazolidine-4,1′-indene]-1-yl)acetamide;
N-benzyl-N-(1-cyclopropylethyl)-2-(2′,4′-dioxo-2,3-dihydrospiro[indene-1,5′-ozazolidine]-3′-yl)acetamide;
N-benzyl-2-(2,5-dioxo-2′,3′-dihydrospiro[imidazolidine-4,1′-indene]-1-yl)-N-(pentan-3-yl)acetamide;
N-benzyl-N-(1-cyclopropylethyl)-2-(3′-methoxy-2,5-dioxo-2′,3′-dihydrospiro[imidazolidine-4,1′-indene]-1-yl)acetamide;
N-benzyl-N-(1-cyclopropylethyl)-2-(5′-(methylthio)-2,5-dioxo-2′,3′-dihydrospiro[imidazolidine-4,1′-indene]-1-yl)acetamide;
N-benzyl-2-(7′-chloro-2,5-dioxo-2′,3′-dihydrospiro[imidazolidine-4,1′-indene]-1-yl)-N-(1-cyclobutylethyl)acetamide,
N-benzyl-N-(1-cyclopropylethyl)-2-(5′-(hydroxymethyl)-2,5-dioxo-2′,3′-dihydrospiro[imidazolidine-4,1′-indene]-1-yl)acetamide;
N-benzyl-N-(1-cyclopropylethyl)-2-(5′-((difluoromethoxy)methyl)-2,5-dioxo-2′,3′-dihydrospiro[imidazolidine-4,1′-indene]-1-yl)acetamide;
N-benzyl-2-(5′-cyclopropyl-2,5-dioxo-2′,3′-dihydrospiro[imidazolidine-4,1′-indene]-1-yl)-N-(1-cyclopropylethyl)acetamide;
N-benzyl-N-(1-cyclobutylethyl)-2-(3′-methyl-2,5-dioxo-2′,3′-dihydrospiro[imidazolidine-4,1′-indene]-1-yl)acetamide;
N-benzyl-N-(1-cyclobutylethyl)-2-(5′-(difluoromethoxy)-2,5-dioxo-2′,3′-dihydrospiro[imidazolidine-4,1′-indene]-1-yl)acetamide;
N-benzyl-N-(1-cyclopropylethyl)-2-(4′-methyl-2,5-dioxo-2′,3′-dihydrospiro[imidazolidine-4,1′-indene]-1-yl)acetamide;
2-(5′-acetamido-2,5-dioxo-2′,3′-dihydrospiro)-N-benzyl-N-(1-cyclopropylethyl)acetamide;
N-benzyl-N-(1-cyclopropylethyl)-2-(5′-methyl-2,5-dioxo-2′,3′-dihydrospiro[imidazolidine-4,1′-indene]-1-yl)acetamide;
1-(2-(benzyl((S)-1-cyclopropylethyl)amino)-2-oxoethyl)-N-(cyanomethyl)-2,5-dioxo-2′,3′-dihydrospiro[imidazolidine-4,1′-indene]-5′-carboxamide;
N-benzyl-N-(1-cyclopropylethyl)-2-(5′-ethynyl-2,5-dioxo-2′,3′-dihydrospiro[imidazolidine-4,1′-indene]-1-yl)acetamide;
N-benzyl-N-(1-cyclopropylethyl)-2-(5′-(3-hydroxyprop-1-ynyl)-2,5-dioxo-2′,3′-dihydrospiro[imidazolidine-4,1′-indene]-1-yl)acetamide;
N-benzyl-N-(1-cyclobutylethyl)-2-(6′-methoxy-2,5-dioxo-2′,3′-dihydrospiro[imidazolidine-4,1′-indene]-1-yl)acetamide;
N-benzyl-N-(1-cyclobutylethyl)-2-(2-imino-5-oxo-2′,3′-dihydrospiro[imidazolidine-4,1′-indene]-1-yl)acetamide;
N-benzyl-N-(dicyclopropylmethyl)-2-(2,5-dioxo-2′,3′-dihydrospiro[imidazolidine-4,1′-indene]-1-yl)acetamide;
N-benzyl-2-(6′-chloro-2,5-dioxo-2′,3′-dihydrospiro[imidazolidine-4,1′-indene]-cyclobutylethyl)acetamide;
N-benzyl-N-(1-cyclopropylethyl)-2-(2,5-dioxo-5′-(1H-pyrazol-5-yl)-2′,3′-dihydrospiro[imidazolidine-4,1′-indene]-1-yl)acetamide;
N-(4-chlorobenzyl)-N—((S)-1-cyclobutylethyl)-2-((S)-2,5-dioxo-2′,3′-dihydrospiro[imidazolidine-4,1′-indene]-1-yl)acetamide;
N-benzyl-N-(1-cyclopropylethyl)-2-(2,5-dioxo-5′-(1H-1,2,3-triazol-4-yl)-2′,3′-dihydrospiro[imidazolidine-4,1′-indene]-1-yl)acetamide;
N-benzyl-N—((S)-1-cyclobutylethyl)-2-((3′R)-3′-hydroxy-2,5-dioxo-2′,3′-dihydrospiro[imidazolidine-4,1′-indene]-yl)acetamide;
N-benzyl-N—((S)-1-cyclobutylethyl)-2-((3′S)-3′-hydroxy-2,5-dioxo-2′,3′-dihydrospiro[imidazolidine-4,1′-indene]-1-yl)acetamide;
N-(4-bromobenzyl)-N—((S)-1-cyclobutylethyl)-2-((S)-2,5-dioxo-2′,3′-dihydrospiro[imidazolidine-4,1′-indene]-1-yl)acetamide;
N-benzyl-N-(1-cyclopropylethyl)-2-(2,2′,5′-trioxo-1,2,6,7-tetrahydrospiro[cyclopenta[b]pyridine-5,4′-imidazolidine]-1′-yl)acetamide;
2-(5′-(1H-imidazol-2-yl)-2,5-dioxo-2′,3′-dihydrospiro[imidazolidine-4,1′-indene]-1-yl)-N-benzyl-N-(1-cyclopropylethyl)acetamide;
N-benzyl-2-(5-bromo-2′,4′-dioxo-2,3-dihydrospiro[indene-1,5′-ozazolidine]-3∝-yl)-N-(1-cyclopropylethyl)acetamide;
N-benzyl-2-(2′,4′-dioxo-2,3-dihydrospiro[indene-1,5′-ozazolidine]-3′-yl)-N-(pentan-3-yl)acetamide;
N-benzyl-N-(1-cyclobutylethyl)-2-(2′,4′-dioxo-2,3-dihydrospiro[indene-1,5′-ozazolidine]-3′-yl)acetamide;
N-(1-cyclopropylethyl)-2-(2′,4′-dioxo-2,3-dihydrospiro[indene-1,5′-ozazolidine]-3′-yl)-N-(4-fluorobenzyl)acetamide;
N-benzyl-N-(1-cyclopropylethyl)-2-(4-methoxy-2′,4′-dioxo-2,3-dihydrospiro[indene-1,5′-ozazolidine]-3′-yl)acetamide;
N—((S)-1-cyclobutylethyl)-2-((S)-2,5-dioxo-2′,3′-dihydrospiro[imidazolidine-4,1′-indene]-1-yl)-N-(4-(methylthio)benzyl)acetamide;
N-benzyl-N-(1-cyclopropylethyl)-2-(2,5-dioxo-5′-(1H-1,2,3-triazol-1-yl)-2′,3′-dihydrospiro[imidazolidine-4,1′-indene]-1-yl)acetamide;
N-benzyl-N-(1-cyclopropylethyl)-2-(2,5-dioxo-5′-(2H-1,2,3-triazol-2-yl)-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(4-fluorobenzyl)acetamide;
N-(4-cyanobenzyl)-N—((S)-1-cyclobutylethyl)-2-((S)-2,5-dioxo-2′,3′-dihydrospiro[imidazolidine-4,1′-indene]-1-yl)acetamide;
4-((N—((S)-1-cyclobutylethyl)-2-(S)-2,5-dioxo-2′,3′-dihydrospiro[imidazolidine-4,1′-indene]-1-yl)acetamido)methyl)benzamide,
N-benzyl-N-(1-cyclopropylethyl)-2-(5′-(methoxymethyl)-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(4-methylbenzyl)acetamide;
N—((S)-1-cyclobutylethyl)-2-((S)-2,5-dioxo-2′,3′-dihydrospiro[imidazolidine-4,1′-indene]-1-yl)-N-(4-methoxybenzyl)acetamide;
N—((S)-1-cyclobutylethyl)-2-((S)-2,5-dioxo-2′,3′-dihydrospiro[imidazolidine-4,1′-indene]-1-yl)-N-(3-methylbenzyl)acetamide;
N—((S)-1-cyclobutylethyl)-2-((S)-2,5-dioxo-2′,3′-dihydrospiro[imidazolidine-4,1′-indene]-1-yl)-N-(2-methylbenzyl)acetamide;
N-((1H-1,2,3-triazol-4-yl)methyl)-N—((S)-1-cyclobutylethyl)-2-((S)-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(4-(methylsulfonyl)benzyl)acetamide;
2-(5-bromo-2′,4′-dioxo-2,3-dihydrospiro[indene-1,5′-ozazolidine]-3′-yl)-N-(1-cyclopropylethyl)-N-(4-fluorobenzyl)acetamide;
N-benzyl-2-(5-cyano-2′,4′-dioxo-2,3-dihydrospiro[indene-1,5′-ozazolidine]-3′-yl)-N-(1-cyclopropylethyl)acetamide;
N-benzyl-N—((S)-1-cyclopropylethyl)-2-(2,3′,5-trioxo-2′,3′-dihydrospiro[imidazolidine-4,1′-indene]-1-yl)acetamide;
N-benzyl-2-(5′-(2-cyanoacetamido)-2,5-dioxo-2′,3′-dihydrospiro[imidazolidine-4,1′-indene]-1-yl)-N—((S)-1-cyclopropylethyl)acetamide;
methyl 1-(2-(benzyl((S)-1-cyclopropylethyl)amino)-2-oxoethyl)-2,5-dioxo-2′,3′-dihydrospiro[imidazolidine-4,1′-indene]-5′-ylcarbamate
N-benzyl-N—((S)-1-cyclopropylethyl)-2-(7′-fluoro-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(1-phenylethyl)acetamide;
N-benzyl-2-(7′-cyano-2,5-dioxo-2′,3′-dihydrospiro[imidazolidine-4,1′-indene]-1-yl)-N—((S)-1-cyclopropylethyl)acetamide;
(S)-2-(5-acetamido-2′,4′-dioxo-2,3-dihydrospiro[indene-1,5′-ozazolidine]-3′-yl)-N-benzyl-N-(1-cyclopropylethyl)acetamide;
3′-(2-(benzyl(1-cyclopropylethyl)amino)-2-oxoethyl)-2′,4′-dioxo-2,3-dihydrospiro[indene-1,5′-ozazolidine]-5-carboxamide;
N-benzyl-N—((S)-1-cyclopropylethyl)-2-(5′-(3-methylureido)-2,5-dioxo-2′,3′-dihydrospiro[imidazolidine-4,1′-indene]-1-yl)acetamide;
N-benzyl-N—((S)-1-cyclopropylethyl)-2-(5′-(3-methyl-3-(methylcarbamoyl)ureido)-2,5-dioxo-2′,3′-dihydrospiro[imidazolidine-4,1′-indene]-1-yl)acetamide;
N-benzyl-N-(1-cyclopropylethyl)-2-(5-methoxy-2′,4′-dioxo-2,3-dihydrospiro[indene-1,5′-ozazolidine]-3′-yl)acetamide;
N—((S)-1-cyclopropylethyl)-2-((S)-2,5-dioxo-2′,3′-dihydrospiro[imidazolidine-4,1′-indene]-1-yl)-N-(4-ethynylbenzyl)acetamide;
N—((S)-1-cyclobutylethyl)-2-(S)-2,5-dioxo-2′,3′-dihydrospiro[imidazolidine-4,1′-indene]-1-yl)-N-((1-methyl-1H-1,2,3-triazol-4-yl)methyl)acetamide;
N-((1-(cyanomethyl)-1H-1,2,3-triazol-4-yl)methyl)-N—((S)-1-cyclobutylethyl)-2-((S)-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(4-ureidobenzyl)acetamide;
N-benzyl-N—((S)-1-cyclopropylethyl)-2-(5-(3-hydroxyprop-1-ynyl)-2′,4′-dioxo-2,3-dihydrospiro[indene-1,5′-ozazolidine]-3′-yl)acetamide;
N-benzyl-N—((S)-1-cyclopropylethyl)-2-(5′-(3-hydroxy-3-methylbut-1-ynyl)-2,5-dioxo-2′,3′-dihydrospiro[imidazolidine-4,1′-indene]-1-yl)acetamide;
N-((1-(2-amino-2-oxoethyl)-1H-1,2,3-triazol-4-yl)methyl)-N—((S)-1-cyclobutylethyl)-2-((S)-2,5-dioxo-2′,3′-dihydrospiro[imidazolidine-4,1′-indene]-1-yl)acetamide;
(S)—N-benzyl-N-(1-cyclopropylethyl)-2-(2,5-dioxo-5′46-oxo-1,6-dihydropyridin-3-yl)-2′,3′-dihydrospiro[imidazolidine-4,1′-indene]-1-yl)acetamide;
N-benzyl-N—((S)-1-cyclopropylethyl)-2-((S)-2,5-dioxo-5′-(1H-pyrazol-3-yl)-2′,3′-dihydrospiro[imidazolidine-4,1′-indene]-1-yl)acetamide;
N-benzyl-N—((S)-1-cyclopropylethyl)-2-(5-(methylsulfonamido)-2′,4′-dioxo-2,3-dihydrospiro[indene-1,5′-ozazolidine]-3′-yl)acetamide;
N-benzyl-N—((S)-1-cyclopropylethyl)-2-(5′-(methylsulfonamido)-2,5-dioxo-2′,3′-dihydrospiro[imidazolidine-4,1′-indene]-1-yl)acetamide;
N-benzyl-2-(5′(2-cyanoacetamido)-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(2,5-dioxo-5′-(2H-tetrazol-5-yl)-2′,3′-dihydrospiro[imidazolidine-4,1′-indene]-1-yl)acetamide;
(S)—N-benzyl-N-(1-cyclopropylethyl)-2-(3′-(hydroxyimino)-2,5-dioxo-2′,3′-dihydrospiro[imidazolidine-4,1′-indene]-1-yl)acetamide;
(S)—N-benzyl-N-(1-cyclopropylethyl)-2-(3′-(methoxyimino)-2,5-dioxo-2′,3′-dihydrospiro[imidazolidine-4,1′-indene]-1-yl)acetamide;
(S)-3′-(2-(benzyl(1-cyclopropylethyl)amino)-2-oxoethyl)-2′,4′-dioxo-2,3-dihydrospiro[indene-1,5′-ozazolidine]-5-carboxamide;
N-benzyl-N—((S)-1-cyclopropylethyl)-2-((S)-2,5-dioxo-5′-(6-oxo-1,6-dihydropyridin-3-yl)-2′,3′-dihydrospiro[imidazolidine-4,1′-indene]-1-yl)acetamide;
N-benzyl-N—((S)-1-cyclopropylethyl)-2-((S)-5′-(3-hydroxyprop-1-ynyl)-2,5-dioxo-2′,3′-dihydrospiro[imidazolidine-4,1′-indene]-1-yl)acetamide;
N-benzyl-N—((S)-1-cyclopropylethyl)-2-((S)-2,5-dioxo-5′-(2H-1,2,3-triazol-4-yl)-2′,3′-dihydrospiro[imidazolidine-4,1′-indene]-1-yl)acetamide;
N-benzyl-N—((S)-1-cyclopropylethyl)-2-(5-(morpholinomethyl)-2′,4′-dioxo-2,3-dihydrospiro[indene-1,5′-ozazolidine]-3′-yl)acetamide;
N-benzyl-N—((S)-1-cyclopropylethyl)-2-(5-(3-methylureido)-2′,4′-dioxo-2,3-dihydrospiro[indene-1,5′-ozazolidine]-3′-yl)acetamide;
N-benzyl-N—((S)-1-cyclopropylethyl)-2-(5′-(3-hydroxypyrrolidin-1-yl)-2,5-dioxo-2′3′-dihydrospiro[imidazolidine-4,1′-indene]-1-yl)acetamide;
N-benzyl-N—((S)-1-cyclopropylethyl)-2-((S)-5′-(3-hydroxy-3-methylbut-1-ynyl)-2,5-dioxo-2′,3′-dihydrospiro[imidazolidine-4′,1′-indene]-1-yl)acetamide;
(S)—N-(3′-(2-(benzyl(1-cyclopropylethyl)amino)-2-oxoethyl)-2′,4′-dioxo-2,3-dihydrospiro[indene-1,5′-ozazolidine]-5-yl)-3-oxobutanamide;
N-benzyl-N—((S)-1-cyclopropylethyl)-2-(5-morpholino-2′,4′-dioxo-2,3-dihydrospiro[indene-1,5′-ozazolidine]-3′-yl)acetamide;
N-benzyl-2-(5-(2-cyanoacetamido)-2′,4′-dioxo-2,3-dihydrospiro[indene-1,5′-ozazolidine]-3′-yl)-N—((S)-1-cyclopropylethyl)acetamide;
N-benzyl-2-((S)-5′-cyano-2,5-dioxo-2′,3′-dihydrospiro[imidazolidine-4,1′-indene]-1-yl)-N—((S)-1-cyclopropylethyl)acetamide;
1-(2-(benzyl-((S)-1-cyclopropylethyl)amino)-2-oxoethyl)-2,5-dioxo-2′,3′-dihydrospiro[imidazolidine-4,1′-indene]-3′-carboxylic acid;
N-benzyl-N—((S)-1-cyclopropylethyl)amino)-2-((S)-5′-(3-methylureido)-2,5-dioxo-2′,3′-dihydrospiro[imidazolidine-4,1′-indene]-1-yl)acetamide;
N-benzyl-N—((S)-1-cyclopropylethyl)-2-(2′,4′-dioxo-5-(2-oxa-6-azaspiro[3.3]heptan-6-yl)-2,3-dihydrospiro[indene-1,5′-ozazolidine]-3′-yl)acetamide;
N-benzyl-N—((S)-1-cyclopropylethyl)-2-(2′,4′-dioxo-5-(piperidin-1-ylmethyl)-2,3-dihydrospiro[indene-1,5′-ozazolidine]-3′-yl)acetamide;
N-benzyl-N—((S)-1-cyclopropylethyl)-2-(2′,4′-dioxo-5-(piperazin-1-ylmethyl)-2,3-dihydrospiro[indene-1,5′-ozazolidine]-3′-yl)acetamide;
N-benzyl-N—((S)-1-cyclopropylethyl)-2-(5-((4-hydroxypiperidin-1-yl)methyl)-2′,4′-dioxo-2,3-dihydrospiro[indene-1,5′-ozazolidine]-3′-yl)acetamide;
(S)-2-amino-N-(3′-(2-(benzyl(1-cyclopropylethyl)amino)-2-oxoethyl)-2′,4′-dioxo-2,3-dihydrospiro[indene-1,5′-ozazolidine]-5-yl)-2-methylpropanamide;
(S)—N-benzyl-N-(1-cyclopropylethyl)-2-(2′,4′-dioxo-5-(piperazin-1-yl)-2,3-dihydrospiro[indene-1,5′-ozazolidine]-3′-yl)acetamide
N-benzyl-N—((S)-1-cyclopropylethyl)-2-(5′-(3-hydroxyazetidin-1-yl)-2,5-dioxo-2′,3′-dihydrospiro[imidazolidine-4,1′-indene]-1-yl)acetamide;
N-benzyl-2-((S)-5′-(2-cyanoacetamido)-2,5-dioxo-2′,3′-dihydrospiro[imidazolidine-4,1′-indene]-1-yl)-N—((S)-1-cyclopropylethyl)acetamide;
1-(2-(benzyl((S)-1-cyclopropylethyl)amino)-2-oxoethyl)-2,5-dioxo-2′,3′-dihydrospiro[imidazolidine-4,1′-indene]-3′-carboxamide;
N-benzyl-2-(3′-cyano-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(5′-(2,4-dioxo-3,4-dihydropyrimidin-1(2H)-yl)-2,5-dioxo-2′,3′-dihydrospiro[imidazolidine-4,1′-indene]-1-yl)acetamide;
N-benzyl-N—((S)-1-cyclopropylethyl)-2-(5-hydroxy-2′,4′-dioxo-2,3-dihydrospiro[indene-1,5′-ozazolidine]-3′-yl)acetamide,
methyl 2-(3′-(2-(benzyl((S)-1-cyclopropylethyl)amino)-2-oxoethyl)-2′,4′-dioxo-2,3-dihydrospiro[indene-1,5′-ozazolidine]-5-yloxy)acetate;
N-benzyl-N—((S)-1-cyclopropylethyl)-2-(2′,4′-dioxo-5-(prop-2-ynyloxy)-2,3-dihydrospiro[indene-1,5′-ozazolidine]-3′-yl)acetamide;
N-(1-(2-(benzyl((S)-1-cyclopropylethylamino)-2-oxoethyl)-2,5-dioxo-2′,3′-dihydrospiro[imidazolidine-4,1′-indene]-5′-yl)acrylamide,
N-benzyl-2-(5′-(2-cyano-1-hydroxyallyl)-2,5-dioxo-2′,3′-dihydrospiro[imidazolidine-4,1′-indene]-1-yl)-N—((S)-1-cyclopropylethyl)acetamide;
(S)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)-3-methylbutanamide;
(2S)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)propanamide;
N-benzyl-N—((S)-1-cyclopropylethyl)-2-(5′-(2,4-dioxo-1,2,3,4-tetrahydropyrimidin-5-yl)-2,5-dioxo-2′,3′-dihydrospiro[imidazolidine-4,1′-indene]-1-yl)acetamide;
1-(2-(benzyl((S)-1-cyclopropylethyl)amino)-2-oxoethyl)-2,5-dioxo-2′,3′-dihydrospiro[imidazolidine-4,1′-indene]-5′-carboxylic acid;
2-(3′-(2-(benzyl((S)-1-cyclopropylethyl)amino)-2-oxoethyl)-2′,4′-dioxo-2,3-dihydrospiro[indene-1,5′-ozazolidine]-5-ylamino)acetic acid;
N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-cyano-3-oxobutanamide;
(S)—N-(1-(2-(benzyl(1-cyclopropylethyl)amino)-2-oxoethyl)-2,5-dioxo-2′,3′-dihydrospiro[imidazolidine-4,1′-indene]-5′-yl)-3-oxobutanamide;
N-(1-(2-(benzyl((S)-1-cyclopropylethyl)amino)-2-oxoethyl)-2,5-dioxo-2′,3′-dihydrospiro[imidazolidine-4,1′-indene]-5′-yl)-2-cyano-3-oxobutanamide;
2-(3′-(2-(benzyl((S)-1-cyclopropylethyl)amino)-2-oxoethyl)-2′,4′-dioxo-2,3-dihydrospiro[indene-1,5′-ozazolidine]-5-yloxy)acetic acid;
N-benzyl-N—((S)-1-cyclopropylethyl)-2-(5-(oxetan-3-ylamino)-2′,4′-dioxo-2,3-dihydrospiro[indene-1,5′-ozazolidine]-3′-yl)acetamide;
N-benzyl-N—((S)-1-cyclopropylethyl)-2-(2′,4′-dioxo-5-(tetrahydro-2H-pyran-4-ylamino)-2,3-dihydrospiro[indene-1,5′-ozazolidine]-3′-yl)acetamide;
N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-hydroxypropanamide
(E)-N-(1-(2-(benzyl((S)-1-cyclopropylethyl)amino)-2-oxoethyl)-2,5-dioxo-2′,3′-dihydrospiro[imidazolidine-4,1′-indene]-5′-yl)-4-(piperidin-1-yl)but-2-enamide;
N-benzyl-2-((S)-2,5-dioxo-2′,3′-dihydrospiro[imidazolidine-4,1′-indene]-1-yl)-N-(1-(oxetan-3-yl)ethyl)acetamide
1-(3′-(2-(benzyl((S)-1-cyclopropylethyl)amino)-2-oxoethyl)-2′,4′-dioxo-2,3-dihydrospiro[indene-1, 5′-ozazolidine]-5-yl)azetidine-3-carboxamide;
2-amino-N-(3′-(2-(benzyl(dicyclopropylmethyl)amino)-2-oxoethyl)-2′,4′-dioxo-2,3-dihydrospiro[indene-1,5′-ozazolidine]-5-yl)-2-methylpropanamide;
N-benzyl-N-(dicyclopropylmethyl)-2-(5-(3-methylureido)-2′,4′-dioxo-2,3-dihydrospiro[indene-1,5′-ozazolidine]-3′-yl)acetamide;
N-benzyl-N—((S)-1-cyclopropylethyl)-2-(5-(2-(dimethylamino)ethoxy)-2′,4′-dioxo-2,3-dihydrospiro[indene-1,5′-ozazolidine]-3′-yl)acetamide;
2-(5-((1H-1,2,3-triazol-4-ylmethoxy)-2′,4′-dioxo-2,3-dihydrospiro[indene-1,5′-ozazolidine]-3′-yl)-N-benzyl-N—((S)-1-cyclopropylethyl)acetamide;
N-benzyl-N—((S)-1-cyclopropylethyl)-2-(2′,4′-dioxo-5-(6-oxo-1,6-dihydropyridin-3-yl)-2,3-dihydrospiro[indene-1,5′-ozazolidine]-3′-yl)acetamide;
N-benzyl-N—((S)-1-cyclopropylethyl)-2-(5-(2-hydroxyethoxy)-2′,4′-dioxo-2,3-dihydrospiro[indene-1,5′-ozazolidine]-3′-yl)acetamide;
3′-(2-(benzyl((S)-1-cyclopropylethyl)amino)-2-oxoethyl)-2′,4′-dioxo-2,3-dihydrospiro[indene-1,5′-ozazolidine]-5-ylboronic acid;
N-benzyl-N—((S)-1-cyclopropylethyl)-2-(5-(3-(hydroxymethyl)azetidin-1-yl)-2′,4′-dioxo-2,3-dihydrospiro[indene-1,5′-ozazolidine]-3′-yl)acetamide;
2-amino-N-((1′S)-1-((4S)-3-benzyl-4-cyclopropyl-2-oxopentyl)-2,5-dioxo-2′,3′-dihydrospiro[imidazolidine-4,1′-indene]-5′-yl)-2-methylpropanamide;
N—((S)-1-cyclopropylethyl)-2-(2′,4′-dioxo-5-(6-oxo-1,6-dihydropyridin-3-yl)-2,3-dihydrospiro[indene-1,5′-ozazolidine]-3′-yl)-N-(4-fluorobenzyl)acetamide;
(S)-2-amino-N—((S)-1-(2-(benzyl((S)-1-cyclopropylethyl)amino)-2-oxoethyl)-2,5-dioxo-2′,3′-dihydrospiro[imidazolidine-4′,1′-indene]-5′-yl)propanamide
1-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)cyclobutanecarboxamide;
N-benzyl-N—((S)-1-cyclopropylethyl)-2-(5′-(2-hydroxy-5-oxo-2,5-dihydrofuran-2-yl)-2,5-dioxo-2′,3′-dihydrospiro[imidazolidine-4,1′-indene]-1-yl)acetamide;
2-(5-(2-cyanoacetamido)-2′,4′-dioxo-2,3-dihydrospiro[indene-1,5′-ozazolidine]-3′-yl)-N—((S)-1-cyclopropylethyl)-N-(4-fluorobenzyl)acetamide;
N—((S)-1-cyclopropylethyl)-N-(4-fluorobenzyl)-2-(5-(3-methylureido)-2′,4′-dioxo-2,3-dihydrospiro[indene-1,5′-ozazolidine]-3′-yl)acetamide;
5′-(azetidin-3-ylmethylamino)-1-((4S)-3-benzyl-4-cyclopropyl-2-oxopentyl)-2′,3′-dihydrospiro[imidazolidine-4,1′-indene]-2,5-dione;
3′-((4S)-3-benzyl-4-cyclopropyl-2-oxopentyl)-5-(1H-pyrazol-5-yl)-2,3-dihydrospiro[indene-1,5′-ozazolidine]-2′,4′-dione;
2-amino-N-(3′-(2-(benzyl((S)-1-cyclopropylethyl)amino)-2-oxoethyl)-4′-oxo-2,3-dihydrospiro[indene-1,5′-ozazolidine]-5-yl)-3-hydroxypropanamide;
(S)—N-(3′-(2-(benzyl(1-cyclopropylethyl)amino)-2-oxoethyl)-2′,4′-dioxo-2,3-dihydrospiro[indene-1,5′-ozazolidine]-5-yl)-2-hydroxy-2-methylpropanamide;
1-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)cyclopropanecarboxamide;
2-amino-N-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)-2-methylpropanamide;
(S)-2-(5-(azetidin-3-ylamino)-2′,4′-dioxo-2,3-dihydrospiro[indene-1,5′-ozazolidine]-3′-yl)-N-benzyl-N-(1-cyclopropylethyl)acetamide;
1-amino-N—((S)-1-(2-(benzyl((S)-1-cyclopropylethyl)amino)-2-oxoethyl)-2,5-dioxo-2′,3′-dihydrospiro[imidazolidine-4,1′-indene]-5′-yl)cyclobutanecarboxamide;
(2R)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)propanamide;
N-benzyl-N—((S)-1-cyclopropylethyl)-2-(2,5-dioxo-5′-(thiazol-2-ylamino)-2′,3′-dihydrospiro[imidazolidine-4,1′-indene]-1-yl)acetamide;
(S)-2-(5′-(azetidin-3-ylamino)-2,5-dioxo-2′,3′-dihydrospiro[imidazolidine-4,1′-indene]-1-yl)-N-benzyl-N-(1-cyclopropylethyl)acetamide;
(S)-2-(3′-(2-(benzyl(1-cyclopropylethyl)amino)-2-oxoethyl)-2′,4′-dioxo-2,3-dihydrospiro[indene-1,5′-ozazolidine]-5-ylcarboxamido)acetic acid;
2-((S)-5-acetamido-2′,4′-dioxo-2,3-dihydrospiro[indene-1,5′-ozazolidine]-3′-yl)-N-benzyl-N—((S)-1-cyclopropylethyl)acetamide;
2-((R)-5-acetamido-2′,4′-dioxo-2,3-dihydrospiro[indene-1,5′-ozazolidine]-3′-yl)-N-benzyl-N—((S)-1-cyclopropylethyl)acetamide;
N-benzyl-N-(1-cyclopropylethyl)-2-(2,5-dioxo-1′,3′-dihydrospiro[imidazolidine-4,2′-indene]-1-yl)acetamide;
N-benzyl-N-(1-cyclopropylethyl)-2-(8′-methoxy-2,5-dioxo-3′,4′-dihydro-1′H-spiro[imidazolidine-4,2′-naphthalene]-1-yl)acetamide;
N-benzyl-N-(1-cyclobutylethyl)-2-(3′,3′-dimethyl-2,5-dioxo-2′,3′-dihydrospiro[imidazolidine-4,1′-indene]-1-yl)acetamide;
N-benzyl-2-[5′-(5-cyano-6-oxo-1,6-dihydropyridin-3-yl)-2,5-dioxo-2′,3′-dihydrospiro[imidazolidine-4,1′-indene]-1-yl]-N-(1-cyclopropylethyl)acetamide;
N-benzyl-2-{5-[(methylcarbamoyl)amino]-3′,5′-dioxo-2,3-dihydrospiro[indene-1,2′-[1,4]ozazolidine]-4′-yl}-N-(pentan-3-yl)acetamide;
2-amino-N-(4′-{[benzyl(pentan-3-yl)carbamoyl]methyl}-3′,5′-dioxo-2,3-dihydrospiro[indene-1,2′-[1,4]ozazolidine]-5-yl)-2-methylpropanamide;
(2R)-2-amino-N-[(4S)-1-({benzyl[(1S)-1-cyclopropylethyl]carbamoyl}methyl)-2,5-dioxo-2′,3′-dihydrospiro[imidazolidine-4,1′-indene]-5′-yl]propanamide;
N-benzyl-N-[(1S)-1-cyclopropylethyl]-2-{5′-[(1H-imidazol-2-yl)amino]-2,5-dioxo-2′,3′-dihydrospiro[imidazolidine-4,1′-indene]-1-yl}acetamide;
1-amino-N-[(4S)-1-({benzyl[(1S)-1-cyclopropylethyl]carbamoyl}methyl)-2,5-dioxo-2′,3′-dihydrospiro[imidazolidine-4,1′-indene]-5′-yl]cyclopropane-1-carboxamide;
2-{5-[(azetidin-3-ylmethyl)amino]-3′,5′-dioxo-2,3-dihydrospiro[indene-1,2′-[1,4]ozazolidine]-4′-yl}-N-benzyl-N-[(1S)-1-cyclopropylethyl]acetamide;
N-benzyl-N-[(1R)-1-cyclopropyl-2,2,2-trifluoroethyl]-2-[(4S)-2,5-dioxo-2′,3′-dihydrospiro[imidazolidine-4,1′-indene]-1-yl]acetamide;
N-benzyl-N-[(1S)-1-cyclopropylethyl]-2-{5′-[(oxetan-3-ylmethyl)amino]-2,5-dioxo-2′,3′-dihydrospiro[imidazolidine-4,1′-indene]-1-yl}acetamide;
N-benzyl-N-[(1S)-1-cyclopropylethyl]-2-{5-[(1,3-dihydroxypropan-2-yl)oxy]-3′,5′-dioxo-2,3-dihydrospiro[indene-1,2′-[1,4]ozazolidine]-4′-yl}acetamide;
1-amino-N-[(4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]cyclobutane-1-carboxamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]azetidine-3-carboxamide;
4-{[4′-({benzyl[(1S)-1-cyclopropylethyl]carbamoyl}methyl)-3′,5′-dioxo-2,3-dihydrospiro[indene-1,2′-[1,4]ozazolidine]-5-ylmethyl]amino}benzoic acid;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-2,3-dihydroxypropanamide;
N-benzyl-2-{5-[(methylcarbamoyl)amino]-3′,5′-dioxo-2,3-dihydrospiro[indene-1,2′-[1,4]ozazolidine]-4′-yl}-N-(oxolan-3-yl)acetamide;
N-benzyl-N-[(1S)-1-cyclopropylethyl]-2-{5-[(1,1-dioxo-1λ⁶-thian-4-yl)amino]-3′,5′-dioxo-2,3-dihydrospiro[indene-1,2′-[1,4]ozazolidine]-4′-yl}acetamide;
N-benzyl-N-[(1S)-1-cyclopropylethyl]-2-{1,1,2′,5′-tetraoxo-2H-spiro[1λ⁶-benzothiophene-3,4′-imidazolidine]-1′-yl}acetamide;
2-{5-[(2R)-2-amino-2-cyclopropylacetamido]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
2-[5′-(azetidin-3-yloxy)-2,5-dioxo-2′,3′-dihydrospiro[imidazolidine-4,1′-indene]-1-yl]-N-benzyl-N-[(1S)-1-cyclopropylethyl]acetamide;
N-(cyclopropylmethyl)-2-{5-[(methylcarbamoyl)amino]-3′,5′-dioxo-2,3-dihydrospiro[indene-1,2′-[1,4]ozazolidine]-4′-yl}-N-[(2-methylphenyl)methyl]acetamide;
N-ethyl-2-{5-[(methylcarbamoyl)amino]-3′,5′-dioxo-2,3-dihydrospiro[indene-1,2′-[1,4]ozazolidine]-4′-yl}-N-[(2-methylphenyl)methyl]acetamide;
N-[(1S)-1-cyclopropylethyl]-2-{3′,5′-dioxo-5-[(pyrrolidin-3-yl)amino]-2,3-dihydrospiro[indene-1,2′-[1,4]ozazolidine]-4′-yl}-N-[(4-fluorophenyl)methyl]acetamide;
2-(5-{[(1-aminocyclopropyl)methyl]amino}-3′,5′-dioxo-2,3-dihydrospiro[indene-1,2′-[1,4]ozazolidine]-4′-yl)-N-[(1S)-1-cyclopropylethyl]-N-[(4-fluorophenyl)methyl]acetamide;
N-benzyl-2-{5-[(methylcarbamoyl)amino]-3′,5′-dioxo-2,3-dihydrospiro[indene-1,2′-[1,4]ozazolidine]-4′-yl}-N-(oxan-4-yl)acetamide;
N-benzyl-N-[(1R)-1-cyclopropyl-2,2,2-trifluoroethyl]-2-{5-[(methylcarbamoyl)amino]-3′,5′-dioxo-2,3-dihydrospiro[indene-1,2′-[1,4]ozazolidine]-4′-yl}acetamide;
N-benzyl-2-{5-[(methylcarbamoyl)amino]-3′,5′-dioxo-2,3-dihydrospiro[indene-1,2′-[1,4]ozazolidine]-4′-yl}-N-(oxan-3-yl)acetamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]ethanediamide;
2-{5-[(azetidin-3-yl)amino]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-benzyl-N-[(1S)-1-cyclopropylethyl]-2-{3′,5′-dioxo-5-[(2-oxopyrrolidin-3-yl)amino]-2,3-dihydrospiro[indene-1,2′-[1,4]ozazolidine]-4′-yl}acetamide;
2-{[4′-({benzyl[(1S)-1-cyclopropylethyl]carbamoyl}methyl)-3′,5′-dioxo-2,3-dihydrospiro[indene-1,2′-[1,4]ozazolidine]-5-yl]carbamoyl}-2,2-dimethylacetic acid;
N-benzyl-N-[(1S)-1-cyclopropylethyl]-2-{5-[(2-hydroxyethyl)amino]-3′,5′-dioxo-2,3-dihydrospiro[indene-1,2′-[1,4]ozazolidine]-4′-yl}acetamide;
(2R)-2-amino-N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]propanamide;
{[4′-({benzyl[(1S)-1-cyclopropylethyl]carbamoyl}methyl)-3′,5′-dioxo-2,3-dihydrospiro[indene-1,2′-[1,4]ozazolidine]-5-yl]carbamoyl}formic acid;
(2S)-2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-hydroxypropanamide;
N-[(1S)-1-cyclopropylethyl]-2-{3′,5′-dioxo-5-[(piperidin-3-yl)amino]-2,3-dihydrospiro[indene-1,2′-[1,4]-ozazolidine]-4′-yl}-N-[(4-fluorophenyl)methyl]acetamide;
N-benzyl-N-[(1S)-1-cyclopropylethyl]-2-{5-[(methylcarbamoyl)amino]-3′,5′-dioxo-2,3-dihydro(3,3-²H₂)spiro[indene-1,2′-[1,4]ozazolidine]-4′-yl}acetamide;
N-[(4-fluorophenyl)methyl]-2-{5-[(methylcarbamoyl)amino]-3′,5′-dioxo-2,3-dihydrospiro[indene-1,2′-[1,4]ozazolidine]-4-yl}-N-[(2S)-1,1,1-trifluoropropan-2-yl]acetamide;
2-amino-N-[4′-({benzyl[(1R)-1-cyclopropyl-2,2,2-trifluoroethyl]carbamoyl}methyl)-3′,5′-dioxo-2,3-dihydrospiro[indene-1,2′-[1,4]ozazolidine]-5-yl]-2-methylpropanamide;
2-{5-[(methylcarbamoyl)amino]-3′,5′-dioxo-2,3-dihydrospiro[indene-1,2′-[1,4]ozazolidine]-4′-yl}-N-[(2-methylphenyl)methyl]-N-(2-methylpropyl)acetamide;
2-{5-[(4-aminooxolan-3-yl)amino]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-[(4-²H)benzyl]-N-[(1S)-1-cyclopropylethyl]-2-{5-[(methylcarbamoyl)amino]-3′,5′-dioxo-2,3-dihydrospiro[indene-1,2′-[1,4]ozazolidine]-4-yl}acetamide;
N-[4-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-2-methyl-2-(methylamino)propanamide;
(2R)-2-amino-N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-benzyl-N-[(1S)-1-cyclopropylethyl]-2-[(1S)-5-[(methylcarbamoyl)amino]-3′,5′-dioxo-2,3-dihydrospiro[indene-1,2′-[1,4]ozazolidine]-4′-yl]acetamide;
N-benzyl-N-[(1S)-1-cyclopropylethyl]-2-[(1R)-5-[(methylcarbamoyl)]amino]-3′,5′-dioxo-2,3-dihydrospiro[indene-1,2′-[1,4]ozazolidine]-4′-yl]acetamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-methylazetidine-3-carboxamide;
N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylazetidine-3-carboxamide;
2-{5-[(2R)-2-amino-2-cyclopropylacetamido]-3′,5′-dioxo-2,3-dihydrospiro[indene-1,2′-[1,4]ozazolidine]-4′-yl}-N-[(4-fluorophenyl)methyl]-N-[(2S)-1,1,1-trifluoropropan-2-yl]acetamide;
N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylazetidine-3-carboxamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-2-(dimethylamino)-2-methylpropanamide;
(2R)-2-amino-N-[4′-({benzyl[(1S)-1-cyclopropylethyl]carbamoyl}methyl)-3′,5′-dioxo-2,3-dihydrospiro[indene-1,2′-[1,4]-ozazolidine]-5-yl]-3-hydroxypropanamide;
N-[(2-amino-1,3-thiazol-5-yl)methyl]-N-[(1S)-1-cyclopropylethyl]-2-[(4S)-2,5-dioxo-2′,3′-dihydrospiro[imidazolidine-4,1′-indene]-1-yl]acetamide;
N-[(1S)-1-cyclopropylethyl]-N-{[2-(dimethylamino)-1,3-thiazol-5-yl]methyl}2-[(4S)-2,5-dioxo-2′,3′-dihydrospiro[imidazolidine-4,1′-indene]-1-yl]acetamide;
N-[(1S)-1-cyclopropylethyl]-N-{[6-(dimethylamino)pyridin-3-yl]methyl}-2-[(4S)-2,5-dioxo-2′,3′-dihydrospiro[imidazolidine-4,1′-indene]-1-yl]acetamide;
2-{5-bromo-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
2-{5-[(3-aminocyclohexyl)amino]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-[(2-amino-1,3-thiazol-4-yl)methyl]-N-[(1S)-1-cyclopropylethyl]-2-[(4S)-2,5-dioxo-2′,3′-dihydrospiro[imidazolidine-4,1′-indene]-1-yl]acetamide;
N-[1-(azetidin-3-yl)ethyl]-N-benzyl-2-{5-[(methylcarbamoyl)amino]-3′,5′-dioxo-2,3-dihydrospiro[indene-1,2′-[1,4]ozazolidine]-4′-yl}acetamide;
N-[(1S)-1-cyclopropylethyl]-N-[(4-fluorophenyl)methyl]-2-{1,1,2′,5′-tetraoxo-2H-spiro[1λ⁶-benzothiophene-3,4′-imidazolidine]-1′-yl}acetamide;
N-[(1S)-1-cyclopropylethyl]-2-[(4S)-2,5-dioxo-2′,3′-dihydrospiro[imidazolidine-4,1′-indene]-1-yl]-N-[(6-hydroxypyridin-3-yl)methyl]acetamide,
N-[(1S)-1-cyclopropylethyl]-2-[(4S)-2,5-dioxo-2′,3′-dihydrospiro[imidazolidine-4,1′-indene]-1-yl]-N-[(6-methoxypyridin-3-yl)methyl]acetamide;
2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-2-methylpropanamide,
N-[(4-fluorophenyl)methyl]-N-[(2S)-1-methoxypropan-2-yl]-2-{5-[(methylcarbamoyl)amino]-3′,5′-dioxo-2,3-dihydrospiro[indene-1,2′-[1,4]ozazolidine]-4′-yl}acetamide;
3-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]oxetane-3-carboxamide;
N-[(1S)-1-cyclopropylethyl]-N-{[2-(dimethylamino)-1,3-thiazol-4-yl]methyl}-2-[(4S)-2,5-dioxo-2′,3′-dihydrospiro[imidazolidine-4,1′-indene]-1-yl]acetamide;
N-benzyl-N-[(1R)-1-cyclopropyl-2,2,2-trifluoroethyl]-2-{1,1,2′,5′-tetraoxo-2H-spiro[1λ⁶-benzothiophene-3,4′-imidazolidine]-1′-yl}acetamide;
N-[(6-amino-5-methoxypyridin-3-yl)methyl]-N-[(1S)-1-cyclopropylethyl]-2-[(4S)-2,5-dioxo-2′,3′-dihydrospiro[imidazolidine-4,1′-indene]-1-yl]acetamide;
N-[(6-aminopyridin-3-yl)methyl]-N-[(1S)-1-cyclopropylethyl]-2-[(4S)-2,5-dioxo-2′,3′-dihydrospiro[imidazolidine-4,1′-indene]-1-yl]acetamide;
N-benzyl-2-{6-bromo-1,1,2′,5′-tetraoxo-2H-spiro[1λ⁶-benzothiophene-3,4′-imidazolidine]-1′-yl}-N-[(1S)-1-cyclopropylethyl]acetamide;
N-benzyl-N-[(1S)-1-cyclopropylethyl]-2-[1,1,2′,5′-tetraoxo-6-(1H-pyrazol-3-yl)-2H-spiro[1λ⁶-benzothiophene-3,4′-imidazolidine]-1′-yl]acetamide;
2-amino-N-[(1S)-4′-({[(4-bromophenyl)methyl][(1S)-1-cyclopropylethyl]carbamoyl}methyl)-3′,5′-dioxo-2,3-dihydrospiro[indene-1,2′-[1,4]ozazolidine]-5-yl]-2-methylpropanamide;
2-amino-N-[(1R)-4′-({[(4-bromophenyl)methyl][(1S)-1-cyclopropylethyl]carbamoyl}methyl)-3′,5′-dioxo-2,3-dihydrospiro[indene-1,2′-[1,4]ozazolidine]-5-yl]-2-methylpropanamide;
2-{5-[(2R)-2-amino-2-(oxetan-3-yl)acetamido]-3′,5′-dioxo-2,3-dihydrospiro[indene-1,2′-[1,4]ozazolidine]-4′-yl}-N-benzyl-N-[(1S)-1-cyclopropylethyl]acetamide;
1-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3-difluorocyclobutane-1-carboxamide;
2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide
(2R)-2-amino-N-[(1S)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1R)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]-N-[(2S)-1,1,1-trifluoropropan-2-yl]acetamide;
N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]-N-[(2S)-1,1,1-trifluoropropan-2-yl]acetamide;
N-[(1S)-1-cyclopropylethyl]-N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]acetamide;
N-[(1S)-1-cyclopropylethyl]-N[((4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]acetamide;
(2S)-2-amino-N-((1S)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1S)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2S)-2-amino-N-[(1R)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1R)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
N-benzyl-N-[(1R)-1-cyclopropyl-2,2,2-trifluoroethyl]-2-[(1S)-5-[(methylcarbamoyl)amino]-3′,5′-dioxo-2,3-dihydrospiro[indene-1,2′-[1,4]ozazolidine]-4′-yl]acetamide;
N-benzyl-N-[(1R)-1-cyclopropyl-2,2,2-trifluoroethyl]-2-[(1R)-5-[(methylcarbamoyl)amino]-3′,5′-dioxo-2,3-dihydrospiro[indene-1,2′-[1,4]ozazolidine]-4′-yl]acetamide;
(2R)-2-amino-N-8 (1S)-4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1R)-4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1S)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
(2R)-2-amino-N-8 (1R)-4′-({[(1R)-1-cyclopropyl-2,2,2-(trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(1R)-1-cyclopropyl-2,2,2-trifluoroethyl]-N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]acetamide;
N-[(1R)-1-cyclopropyl-2,2,2-trifluoroethyl]-N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]acetamide;
N-[(1S)-4′-({[(1R)-1-cyclopropyl-2,2,2-trifluoroethyl][(4-fluorophenyl))methyl]carbamoyl}methyl)-3′,5′-dioxo-2,3-dihydrospiro[indene-1,2′-[1,4]ozazolidine]-5-yl]azetidine-3-carboxamide;
N-[(1R)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
(2R)-2-amino-N-[4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-8 (1S)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
N-[(1R)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
(2S)-2-amino-N-[4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide
(2S)-2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]azetidine-3-carboxamide;
N-benzyl-N-[1-(1-methylazetidin-3-yl)ethyl]-2-{5-[(methylcarbamoyl)amino]-3′,5′-dioxo-2,3-dihydrospiro[indene-1,2′-[1,4]ozazolidine]-4′-yl}acetamide;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1R)-3′-(2-{[(1R)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-benzyl-2-[(1R))-5-bromo-2′,4′-dioxo-2,3-dihydro-3′H-spiro[indene-1,5′-[1,3]ozazolidin]-3′-yl]-N-[(1S)-1-cyclopropylethyl]acetamide;
2-[(1S)-5-amino-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-[(1R)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-benzyl-N-[(1S)-1-cyclopropylethyl]acetamide
N-[(1S)-3′-(2-{[(1R)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide
N-[(1R)-1-cyclopropylethyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-(5′-bromo-2,5-dioxo-1′,3′-dihydro-1H-spiro[imidazolidine-1,2′-inden]-1-yl)-N-[(1S)-1-cyclopropylethyl]-N-(4-fluorobenzyl)acetamide;
2-(6-bromo-1,1-dioxido-2′,5′-dioxo-1′H-spiro[1-benzothiophene-3,4′-imidazolidin]-1′-yl)-N-[(1S)-1-cyclopropylethyl]-N-(4-fluorobenzyl)acetamide;
N-[(1R)-1-cyclopropylethyl]-N-(4-fluorobenzyl)-2-{(1S)-5-[(methylcarbamoyl)amino]-2′,4′-dioxo-2,3-dihydro-3′H-spiro[indene-1,5′-[1,3]ozazolidin]-3′-yl}acetamide;
N-[(1R)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide
N-[(1S)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]azetidine-1-carboxylate
N-[(1S)-1-cyclopropylethyl]-2-[2,5-dioxo-5′-(6-oxo-1,6-dihydropyridin-3-yl)-1′,3′-dihydro-1H-spiro[imidazolidine-4,2′-inden]-1-yl]-N-(4-fluorobenzyl)acetamide;
2-(5′-amino-2,5-dioxo-1′,3′-dihydro-1H-spiro[imidazolidine-4,2′-inden]-1-yl)-N-[(1S)-1-cyclopropylethyl]-N-(4-fluorobenzyl)acetamide;
2-[(1R)-5-(6-aminopyridin-3-yl)-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-{(1R)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-[(1S)-1-cyclopropylethyl]-N-(4-fluorobenzyl)acetamide;
N-[(1S)-1-cyclopropylethyl]-2-[(1R)-2′,4′-dioxo-5-(pyridin-3-yl)-2,3-dihydro-3′H-spiro[indene-1,5′-[1,3]ozazolidin]-3′-yl]-N-(4-fluorobenzyl)acetamide
N-[(1S)-1-cyclopropylethyl]-2-[1,1-dioxido-2′,5′-dioxo-6-(6-oxo-1,6-dihydropyridin-3-yl)-1′H-spiro[1-benzothiophene-3,4′-imidazolidin]-1′-yl]-N-(4-fluorobenzyl)acetamide;
N-benzyl-2-(7-bromo-2′,4′-dioxo-2,3-dihydro-3′H-spiro[chromene-4,5′-[1,3]ozazolidin]-3′-yl)-N-[(1S)-1-cyclopropylethyl]acetamide;
2-[(1R)-5-(azetidin-3-yl)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
2-[(1R)-5-(1-acetylazetidin-3-yl)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-benzyl-N-[(1S)-1-cyclopropylethyl]-2-[(1R)-5-(1-methylazetidin-3-yl)-2′,4′-dioxo-2,3-dihydro-3′H-spiro[indene-1,5′-[1,3]ozazolidin]-3′-yl]acetamide;
3-[(1R)-3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]-N-methylazetidine-1-carboxamide;
N-benzyl-2-{(1R)-5-[1-(cyclopropylcarbonyl)azetidin-3-yl]-2′,4′-dioxo-2,3-dihydro-3′H-spiro[indene-1,5′-[1,3]ozazolidin]-3′-yl}-N-[(1S)-1-cyclopropylethyl]acetamide;
N-benzyl-N-[(1S)-1-cyclopropylethyl]-2-{(1R)-5-[1-(2-hydroxy-2-methylpropanoyl)azetidin-3-yl]-2′,4′-dioxo-2,3-dihydro-3′H-spiro[indene-1,5′-[1,3]ozazolidin]-3′-yl}acetamide;
N-benzyl-N-[(1S)-1-cyclopropylethyl]-2-{(1R)-5-[1-(methylsulfonyl)azetidin-3-yl]2′,4′-dioxo-2,3-dihydro-3′H-spiro[indene-1,5′-[1,3]ozazolidin]-3′-yl}acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]cyclobutanecarboxamide;
3-ethoxy-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-5-oxo-D-prolinamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-5-oxo-L-propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]cyclopropanecarboxamide;
N-(4-fluorobenzyl)-2-{(1R)-5-[(methoxyacetyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(1R)-5-[(ethoxyacetyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-3-methylbutanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylpropanamide;
2-[(1R)-5-(acetylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(7-amino-2′,4′-dioxo-2,3-dihydro-3′H-spiro[chromene-4,5′-[1,3]ozazolidin]-3′-yl)-N-benzyl-N-[(1S)-1-cyclopropylethyl]acetamide;
2-(6-amino-1,1-dioxido-2′,5′-dioxo-1′H-spiro[1-benzothiophene-3,4′-imidazolidin]-1′-yl)-N-[(1S)-1-cyclopropylethyl]-N-(4-fluorobenzyl)acetamide;
N-[(1S)-1-cyclopropylethyl]-N-(4-fluorobenzyl)-2-{5′-[(methylcarbamoyl)amino]-2,5-dioxo-1′,3′-dihydro-1H-spiro[imidazolidine-4,2′-inden]-1-yl}acetamide;
N-(4-fluorobenzyl)-2-[(1R)-5-{[(2-methoxyethyl)(methyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[(dimethylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-{[methyl(2-methylpropyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(cyclopropylmethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(cyclobutylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-({[(2R)-1-hydroxy-3-methylbutan-2-yl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-({[(2S)-tetrahydrofuran-2-ylmethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-({[(2R)-tetrahydrofuran-2-ylmethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-({[2-(propan-2-yloxy)ethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(cyclopropylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-{[ethyl(methyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(diethylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyrrolidine-1-carboxamide;
2-[(1R)-5-({[2-(dimethylamino)ethyl](methyl)carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N²-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-L-leucinamide;
N-(4-fluorobenzyl)-2-[(1R)-5-{[(2-hydroxyethyl)(propyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(2-methoxyethyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(3-hydroxypropyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-({[(2S)-1-hydroxy-3-methylbutan-2-yl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[methyl(propan-2-yl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(2-cyanoethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-{[methyl(propyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[5′-(acetylamino)-2,5-dioxo-1,3′-dihydro-1H-spiro[imidazolidine-4,2′-inden]-1-yl]-N-[(1S)-1-cyclopropylethyl]-N-(4-fluorobenzyl)acetamide;
N-[(1S)-1-cyclopropylethyl]-N-(4-fluorobenzyl)-2-{6-[(methylcarbamoyl)amino]-1,1-dioxido-2′,5′-dioxo-1′H-spiro[1-benzothiophene-3,4′-imidazolidin]-1′-yl}acetamide;
N-(4-fluorobenzyl)-2-(1′-methyl-2,5-dioxo-2′,3′-dihydro-1H,1′H-spiro[imidazolidine-4,4′-quinolin]-1-yl)-N-[(2S)-1,1,1-trifluoropropan-2-yl]acetamide;
N-benzyl-N-[(1S)-1-cyclopropylethyl]-2-{7′-[(methylcarbamoyl)amino]-2′,4′-dioxo-2,3-dihydro-3′H-spiro[chromene-4,5′-[1,3]ozazolidin]-3′-yl}acetamide;
2-[7-(acetylamino)-2′,4′-dioxo-2,3-dihydro-3′H-spiro[chromene-4,5′-[1,3]ozazolidin]-3′-yl]-N-benzyl-N-[(1S)-1-cyclopropylethyl]acetamide;
N-[(1S)-1-cyclopropylethyl]-N-(4-fluorobenzyl)-2-(3′-hydroxy-2,5-dioxo-2′,3′-dihydro-1H-spiro[imidazolidine-4,1′-inden]-1-yl)acetamide;
N-[(1S)-1-cyclopropylethyl]-2-[(1R)-2′,4′-dioxo-5-(pyridin-4-yl)-2,3-dihydro-3′H-spiro[indene-1,5′-[1,3]ozazolidin]-3′-yl]-N-(4-fluorobenzyl)acetamide;
N-[(1S)-1-cyclopropylethyl]-N-(4-fluorobenzyl)-2-[(1R)-5-(6-hydroxypyridazin-3-yl)-2′,4′-dioxo-2,3-dihydro-3′H-spiro[indene-1,5′-[1,3]ozazolidin]-3′-yl]acetamide;
N-[(1S)-1-cyclopropylethyl]-2-[(1R)-2′,4′-dioxo-5-(1H-pyrazol-4-yl)-2,3-dihydro-3′H-spiro[indene-1,5′-[1,3]ozazolidin]-3′-yl]-N-(4-fluorobenzyl)acetamide;
N-(4-fluorobenzyl)-N-[(2S)-1,1,1-trifluoropropan-2-yl]-2-(2,3′,5-trioxo-2′,3′-dihydro-1H-spiro[imidazolidine-4,1′-inden]-1-yl)acetamide;
N-[(1S)-1-cyclopropylethyl]-N-(4-fluorobenzyl)-2-(1′-methyl-2,5-dioxo-2′,3′-dihydro-1H,1′H-spiro[imidazolidine-4,4′-quinolin]-1-yl)acetamide
N-(4-fluorobenzyl)-2-[(3′E)-3′-(hydroxyimino)-2,5-dioxo-2′,3′-dihydro-1H-spiro[imidazolidine-4,1′-inden]-1-yl]-N-[(2S)-1,1,1-trifluoropropan-2-yl]acetamide;
N-[(1S)-1-cyclopropylethyl]-N-(4-fluorobenzyl)-2-(2,3′,5-trioxo-2′,3′-dihydro-1H-spiro[imidazolidine-4,1′-inden]-1-yl)acetamide;
N-benzyl-N-[(1S)-1-cyclopropylethyl]-2-[2′,4′-dioxo-7-(6-oxo-1,6-dihydropyridin-3-yl)-2,3-dihydro-3′H-spiro[chromene-4,5′-[1,3]ozazolidin]-3′-yl]acetamide;
N-(4-fluorobenzyl)-2-(3′-hydroxy-2,5-dioxo-2′,3′-dihydro-1H-spiro[imidazolidine-4,1′-inden]-1-yl)-N-[(2S)-1,1,1-trifluoropropan-2-yl]acetamide;
2-{(1R)-2′,4′-dioxo-5-[(tetrahydrofuran-2-ylmethyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[(cyclopentylmethyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[(2,2-dimethylbutyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-2′,4′-dioxo-5-[(tetrahydrofuran-3-ylmethyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-{(1R)-5-[(2-methylpropyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-[6-(acetylamino)-1,1-dioxido-2′,5′-dioxo-1′H-spiro[1-benzothiophene-3,4′-imidazolidin]-1′-yl]-N-[(1S)-1-cyclopropylethyl]-N-(4-fluorobenzyl)acetamide;
tert-butyl [(2R)-1-{[3′-(2-{benzyl[(1S)-1-cyclopropylethyl]amino}-2-oxoethyl)-2′,4′-dioxo-2,3-dihydrospiro[chromene-4,5′-[1,3]ozazolidin]-7-yl]amino}-3-methyl-1-oxobutan-2-yl]carbamate;
N-[3′-(2-{benzyl[(1S)-1-cyclopropylethyl]amino}-2-oxoethyl)-2′,4′-dioxo-2,3-dihydrospiro[chromene-4,5′-[1,3]ozazolidin]-7-yl]-D-valinamide;
2-[(1R)-6-amino-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
2-[(1R)-6-(acetylamino)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-benzyl-N-[(1S)-1-cyclopropylethyl]-2-{(1R)-6-[(methylcarbamoyl)amino]-2′,4′-dioxo-2,3-dihydro-3′H-spiro[indene-1,5′-[1,3]ozazolidin]-3′-yl}acetamide;
2-[(1S)-6-amino-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
2-[(1S)-6-(acetylamino)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-benzyl-N-[(1S)-1-cyclopropylethyl]-2-{(1S)-6-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-2-[(1R)-2′,4′-dioxo-5-(pyridin-2-yl)-2,3-dihydro-3′H-spiro[indene-1,5′-[1,3]ozazolidin]-3′-yl]-N-(4-fluorobenzyl)acetamide;
2-(7′-bromo-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
N-[(1S)-1-cyclopropylethyl]-N-(3-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-(3′-fluoro-2,5-dioxo-2′,3′-dihydro-1H-spiro[imidazolidine-4,1′-inden]-1-yl)-N-[(2S)-1,1,1-trifluoropropan-2-yl]acetamide;
2-(3′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[3′-(methylamino)-2,5-dioxo-2′,3′-dihydro-1H-spiro[imidazolidine-4,1′-inden]-1-yl]-N-[(2S)-1,1,1-trifluoropropan-2-yl]acetamide;
N-[(1S)-1-cyclopropyl-2,2,2-trifluoroethyl]-N-(4-fluorobenzyl)-2-{7′-[(methylcarbamoyl)amino]-2,4-dioxo-2′,3′-dihydro-3′H-spiro[1,3-ozazolidine-5,4′-thiochromen]-3-yl}acetamide;
2-[(1R)-5-(carbamoylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(7′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
N-[(1S)-1-cyclopropyl-2,2,2-trifluoroethyl]-N-(4-fluorobenzyl)-2-{7′-[(methylcarbamoyl)amino]-1′,1′-dioxido-2,4-dioxo-2′,3′-dihydro-3′H-spiro[1,3-ozazolidine-5,4′-thiochromen]-3-yl}acetamide;
2-(6-bromo-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide;
N-(4-(fluorobenzyl)-2-((R)-5-(3-(N-methylsulfamoyl)ureido)-2′,4′-dioxo-2,3-dihydrospiro[indene-1,5′-ozazolidine]-3′-yl)-N—((S)-1,1,1-trifluoropropan-2-yl)acetamide
methyl {[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}sulfamate;
2-((R)-5-(3-(N-(cyclopropylmethyl)sulfamoyl)ureido)-2′,4′-dioxo-2,3-dihydrospiro[indene-1,5′-ozazolidine]-3′-yl)-N-(4-fluorobenzyl)-N—((S)-1,1,1-trifluoropropan-2-yl)acetamide
N-[(1S)-1-cyclopropylethyl]-N-(4-fluorobenzyl)-2-{7′-[(methylcarbamoyl)amino]-2,5-dioxo-3′,4′-dihydro-1H,1′H-spiro[imidazolidine-4,2′-naphthalen]-1-yl}acetamide;
2-[7′-(acetylamino)-2,5-dioxo-3′,4′-dihydro-1H,1′H-spiro[imidazolidine-4,2′-naphthalen]-1-yl]-N-[(1S)-1-cyclopropylethyl]-N-(4-fluorobenzyl)acetamide;
(1R)-3′-(2-{benzyl[(1S)-1-cyclopropylethyl]amino}-2-oxoethyl)-N-(2-methylpropyl)-2′,4′-dioxo-2,3-dihydrospiro[indene-1,5′-[1,3]ozazolidine]-5-carboxamide;
N-[(1S)-1-cyclopropylethyl]-N-(2-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,3-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}(acetamide;
N-(2-chloro-4-fluorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3-chloro-4-fluorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-[4-(trifluoromethyl)benzyl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1-methyl-1H-pyrazole-4-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1-methyl-1H-pyrazole-3-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-4-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-2-carboxamide;
2-(5-bromo-4-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
2-(5-bromo-6-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
(1R)-3′-(2-{benzyl[(1S)-1-cyclopropylethyl]amino}-2-oxoethyl)-N-[(2R)-1-hydroxy-3-methylbutan-2-yl]-2′,4′-dioxo-2,3-dihydrospiro[indene-1,5′-[1,3]ozazolidine]-5-carboxamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-[(5-methyl-1,3,4-thiadiazol-2-yl)methyl]acetamide;
N-(4-fluorobenzyl)-2-(2′-methyl-2,5-dioxo-2′,3′-dihydro-1H-spiro[imidazolidine-4,1′-inden]-1-yl)-N-[(2S)-1,1,1-trifluoropropan-2-yl]acetamide;
2-(5-amino-4-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{4-fluoro-5-[(methylcarbamoyl)amino]-2′,4′-dioxo-2,3-dihydro-3′H-spiro[indene-1,5′-[1,3]ozazolidin]-3′-yl}acetamide;
N-(4-cyanobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3-methylbenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,4-dichlorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-methoxybenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3-methoxybenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-methylbenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-[(1R)-5-{[(1-methyl-1H-pyrazol-4-yl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-[1-(2-{[(1S)-1-cyclopropylethyl](4-fluorobenzyl)amino}-2-oxoethyl)-2,5-dioxo-3′,4′-dihydro-1′H-spiro[imidazolidine-4,2′-naphthalen]-7′-yl]-5-oxo-D-prolinamide;
2-{7′-[1-(2-amino-2-oxoethyl)-1H-pyrazol-4-yl]-2′,4′-dioxo-2,3-dihydro-3′H-spiro[chromene-4,5′-[1,3]ozazolidin]-3′-yl}-N-benzyl-N-[(1S)-1-cyclopropylethyl]acetamide;
2-{7′-[1-(2-amino-2-oxoethyl)-1H-pyrazol-4-yl]-2,5-dioxo-3′,4′-dihydro-1H,1′H-spiro[imidazolidine-4,2′-naphthalen]-1-yl}-N-[(1S)-1-cyclopropylethyl]-N-(4-fluorobenzyl)acetamide;
2-{(1R)-2′,4′-dioxo-5-[(phenylcarbamoyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-2′,4′-dioxo-5-[(pyridin-3-ylcarbamoyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-(5′-bromo-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-(5-amino-6-fluoro-2′,4′-dioxo-2,3-dihydro-3′H-spiro[indene-1,5′-[1,3]ozazolidin]-3′-yl)-N-(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{6-fluoro-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetamide;
N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(dimethylamino)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(4-methylpyrimidin-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[5-(difluoromethyl)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(6-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1R)-5-[1-(2-methylpropyl)-1H-pyrazol-4-yl]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(1R)-5-[4-(difluoromethyl)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(3-fluoropyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(1,3-thiazol-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-N-methylbenzamide;
2-{(1R)-5-[2-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(5-cyanopyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(1-methyl-1H-1,2,4-triazol-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(5-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-(6-cyanopyridin-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(5-fluoro-3-methylpyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(5-methyl-1,3,4-thiadiazol-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(4-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(6-methoxypyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[2-(dimethylamino)pyrimidin-5-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(acetylamino)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(1-methyl-1H-1,2,4-triazol-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-cyclopropyl-5-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]furan-2-carboxamide;
N-(4-fluorobenzyl)-2-[(1R)-5-(5-fluoropyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
4-fluoro-3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
5-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-N-methylpyridine-2-carboxamide;
6-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-3-carboxamide;
N-(4-fluorobenzyl)-2-[(1R)-5-(2-methoxypyrimidin-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
6-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-2-carboxamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]thiophene-3-carboxamide;
2-{(1R)-5-[4-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(1,3-dimethyl-2,4-dioxo-1,2,3,4-tetrahydropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4-cyanopyridin-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-4-carboxamide;
2-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
2-{(1R)-5-[3-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(6-cyano-5-methoxypyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(1-methyl-1H-pyrazol-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(1,3,5-trimethyl-1H-pyrazol-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(3,4-difluorophenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2,5-difluoro-4-methoxyphenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyrimidin-5-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-2′,4′-dioxo-5-[5-(trifluoromethyl)pyridin-3-yl]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-2′,4′-dioxo-5-(1,3-thiazol-2-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2-cyanopyridin-4-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyrazin-2-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[4-(carbamoylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(2-cyanopyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(6-cyano-5-fluoropyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′]-[1,3]ozazolidin]-5-ylbenzamide;
N-(4-fluorobenzyl)-N-[(2S)-1,1,1-trifluoropropan-2-yl]-2-(2,3,5-trioxo-2′,3′-dihydro-1H,1′H-spiro[imidazolidine-4,4′-quinolin]-1-yl)acetamide;
2-(2,5-dioxo-1H-spiro[imidazolidine-4,1′-inden]-1-yl)-N-(4-fluorobenzyl)-N-[(2S)-1,1,1-trifluoropropan-2-yl]acetamide;
2-{5′-[1-(2-amino-2-oxoethyl)-1H-pyrazol-4-yl]-2,3′,5-trioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-N-[(2S)-1,1,1-trifluoropropan-2-yl]-2-[2,3′,5-trioxo-5′-(6-oxo-1,6-dihydropyridin-3-yl)-2′,3′-dihydro-1H-spiro[imidazolidine-4,1′-inden]-1-yl]acetamide;
2-[(1R)-5-{[(4-aminophenyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1S)-1-cyclopropylethyl]-2-[(4R)-2,5-dioxo-2′,3′-dihydro-1H-spiro[imidazolidine-4,1′-inden]-1-yl]-N-(4-fluorobenzyl)acetamide;
N-[(1S)-1-cyclopropylethyl]-2-[(4S)-2,5-dioxo-2′,3′-dihydro-1H-spiro[imidazolidine-4,1′-inden]-1-yl]-N-(4-fluorobenzyl)acetamide;
2-(7′-bromo-1′-methyl-2,5-dioxo-2′,3′-dihydro-1H,1′H-spiro[imidazolidine-4,4′-quinolin]-1-yl)-N-[(1S)-1-cyclopropylethyl]-N-(4-fluorobenzyl)acetamide;
N-(4-fluorobenzyl)-2-{6-[(methylcarbamoyl)amino]-2′,4′-dioxo-3,4-dihydro-2H,3′H-spiro[naphthalene-1,5′-[1,3]ozazolidin]-3′-yl}-N-[(2S)-1,1,1-trifluoropropan-2-yl]acetamide;
2-[5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(pyrimidin-5-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(pyrimidin-2-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-thiazol-5-ylmethyl)acetamide;
N-(4-fluorobenzyl)-2-(4′-hydroxy-2,5-dioxo-3′,4′-dihydro-1H,2′H-spiro[imidazolidine-4,1′-naphthalen]-1-yl)-N-[(2S)-1,1,1-trifluoropropan-2-yl]acetamide;
2-(6-{1-[2-(dimethylamino)-2-oxoethyl]-1H-pyrazol-4-yl}-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide;
2-[5′-(acetylamino)-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]prolinamide;
N²-(4-fluorobenzyl)-N²-({(1R)-5-[(methylcarbamoyl)amino]-2′,4′-dioxo-2,3-dihydro-3′H-spiro[indene-1,5′-[1,3]ozazolidin]-3′-yl}acetyl)-L-alaninamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1H-pyrazole-5-carboxamide;
2-[5′-(acetylamino)-2,5-dioxo-2′,3′-dihydro-1H-spiro[imidazolidine-4,1′-inden]-1-yl]-N-[(1S)-1-cyclopropylethyl]-N-(4-fluorobenzyl)acetamide;
N-(4-fluorobenzyl)-2-[(1R)-5-{[(methylsulfamoyl)acetyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-(fluorobenzyl)-2-[(1R)-5-{[(methylsulfonyl)acetyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(dimethylsulfamoyl)acetyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-2′,4′-dioxo-5-[(sulfamoylacetyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-(5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide;
(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidine-1-carboxamide
N-benzyl-N-ethyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-(3′-fluoro-5′-{1-[2-(methylamino)-2-oxoethyl]-1H-pyrazol-4-yl}-2,5-dioxo-2′,3′-dihydro-1′H-spiro[imidazolidine-4,1′-inden]-1-yl)-N-[(2S)-1,1,1-trifluoropropan-2-yl]acetamide;
2-(6′-bromo-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
methyl N′-cyano-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamimidothioate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanamide;
2-(6′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
N-(4-fluorobenzyl)-2-{(1R)-5-[(methylcarbamothioyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(tetrahydro-2H-pyran-4-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-oxazol-4-ylmethyl)acetamide;
2-(7′-amino-1′-methyl-2,5-dioxo-2′,3′-dihydro-1H,1′H-spiro[imidazolidine-4,4′-quinolin]-1-yl)-N-[(1S)-1-cyclopropylethyl]-N-(4-fluorobenzyl)acetamide;
2-[(1R)-5-(5-cyanothiophen-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4-cyano-3-fluorophenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(5-cyanothiophen-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1S)-1-cyclopropylethyl]-N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}acetamide;
N-[(1S)-1-cyclopropylethyl]-N-(4-fluorobenzyl)-2-{6′-[(methylcarbamoyl)amino]-2,5-dioxo-3′,4′-dihydro-1H,1′H-spiro[imidazolidine-4,2′-naphthalen]-1-yl}acetamide;
2-[6′-(acetylamino)-2,5-dioxo-3′,4′-dihydro-1H,1′H-spiro[imidazolidine-4,2′-naphthalen]-1-yl]-N-[(1S)-1-cyclopropylethyl]-N-(4-fluorobenzyl)acetamide;
2-[(1R)-5-(N″-cyano-N′-methylcarbamimidamido)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4,6-difluoropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[2-(acetylamino)-5-methylpyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(5-fluoropyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(furan-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(2-oxo-2,3-dihydro-1H-benzimidazol-5-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(4-methylpyrimidin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-2′,4′-dioxo-5-[1-(tetrahydro-2H-pyran-2-yl)-1H-pyrazol-3-yl]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(5-fluoro-6-methylpyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[6-(difluoromethyl)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(difluoromethyl)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(furan-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[1-(ethoxymethyl)-1H-imidazol-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(5-cyano-1,2-dimethyl-6-oxo-1,6-dihydropyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]thiophene-2-carboxamide;
2-[(1R)-5-(2,6-dioxo-1,2,5,6-tetrahydropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2,4-dimethyl-1,3-thiazol-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[1-(cyanomethyl)-3,5-dimethyl-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[3-(carbamoylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-[3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)-2,2-dimethylpropyl]propanamide;
(1R)-3′-(2-{benzyl[(1S)-1-cyclopropylethyl]amino}-2-oxoethyl)-N-methyl-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(propan-2-yl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(2-methoxyethyl)-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(tetrahydrofuran-2-ylmethyl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(tetrahydrofuran-3-ylmethyl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(cyanomethyl)-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(3-hydroxypropyl)-2′,4′-dioxo-2,3-dihydrospiro[indene-1,5′-[1,3]ozazolidine]-5-carboxamide;
3′-{2-[benzyl(1-cyclopropylethyl)amino]-2-oxoethyl}-N-(1-hydroxy-3-methylbutan-2-yl)-2′,4′-dioxo-2,3-dihydrospiro[indene-1,5′-[1,3]ozazolidine]-5-carboxamide;
N-(1-amino-4-methyl-1-oxopentan-2-yl)-3′-{2-[benzyl(1-cyclopropylethyl)amino]-2-oxoethyl}-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-[2-(diethylamino)ethyl]-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-[3-(1H-imidazol-1-yl)propyl]-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-[3-(2-oxopyrrolidin-1-yl)propyl]-2,3-dihydrospiro[indene-1,5′-[1,3]ozazolidine]-5-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]tetrahydropyrimidine-1(2H)-carboxamide;
3-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]tetrahydropyrimidine-1(2H)-carboxamide;
2-(6-amino-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide;
N-(4-fluorobenzyl)-2-(6-{1-[2-(methylamino)-2-oxoethyl]-1H-pyrazol-4-yl}-1,1-dioxido-2′,5′-dioxo-1′H-spiro[1-benzothiophene-3,4′-imidazolidin]-1′-yl)-N-[(2S)-1,1,1-trifluoropropan-2-yl]acetamide;
N-(4-fluorobenzyl)-2-{(1R)-5-[1-(2-hydroxyethyl)-1H-pyrazol-4-yl]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-phenylacetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N,N-dibenzyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl {4-[1-(2-{(4-fluorobenzyl)[(2S)-1,1,1-trifluoropropan-2-yl]amino}-2-oxoethyl)-2,3′,5-trioxo-2′,3′-dihydrospiro[imidazolidine-4,1′-inden]-5′-yl]-1H-pyrazol-1-yl}acetate;
N-(4-fluorobenzyl)-2-{3′-fluoro-5′-[1-(2-methylpropyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
{4-[1-(2-{(4-fluorobenzyl)[(2S)-1,1,1-trifluoropropan-2-yl]amino}-2-oxoethyl)-2,3′,5-trioxo-2′,3′-dihydrospiro[imidazolidine-4,1′-inden]-5′-yl]-1H-pyrazol-1-yl}acetic acid;
N-benzyl-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
(2R)-3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-hydroxy-2-methylpropanamide;
(2S)-3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-hydroxy-2-methylpropanamide;
2-[(1R)-2′,4′-dioxo-5-({[3-(pyrrolidin-1-ylmethyl)phenyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-({[3-(morpholin-4-ylmethyl)phenyl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N³-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-N-methyl-beta-alaninamide;
N-[3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)phenyl]propanamide;
2-(5′-{1-[2-(dimethylamino)-2-oxoethyl]-1H-pyrazol-4-yl}-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-(5′-{1-[2-(3-hydroxyazetidin-1-yl)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide;
2-(6-bromo-2′,4,4′-trioxo-3,4-dihydro-2H,3′H-spiro[naphthalene-1,5′-[1,3]ozazolidin]-3′-yl)-N-[(1S)-1-cyclopropyl-2,2,2-trifluoroethyl]-N-(4-fluorobenzyl)acetamide;
N-(4-fluorobenzyl)-2-{6-[(methylcarbamoyl)amino]-1,1-dioxido-2′,5′-dioxo-1′H-spiro[1-benzothiophene-3,4′-imidazolidin]-1′-yl}-N-[(2S)-1,1,1-trifluoropropan-2-yl]acetamide;
N-(2-chlorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)acetamide;
N-[(1S)-1-cyclopropyl-2,2,2-trifluoroethyl]-N-(4-fluorobenzyl)-2-{6-[(methylcarbamoyl)amino]-2′,4,4′-trioxo-3,4-dihydro-2H,3′H-spiro[naphthalene-1,5′-[1,3]ozazolidin]-3′-yl}acetamide;
N-(4-fluorobenzyl)-2-{(1S)-6-[(methylcarbamoyl)amino]-2′,4′-dioxo-3,4-dihydro-2H,3′H-spiro[naphthalene-1,5′-[1,3]ozazolidin]-3′-yl}-N-[(2S)-1,1,1-trifluoropropan-2-yl]acetamide;
N-(4-fluorobenzyl)-2-{(1R)-6-[(methylcarbamoyl)amino]-2′,4′-dioxo-3,4-dihydro-2H,3′H-spiro[naphthalene-1,5′-[1,3]ozazolidin]-3′-yl}-N-[(2S)-1,1,1-trifluoropropan-2-yl]acetamide;
2-[(1R)-5-(4,5-dihydro-1H-imidazol-2-ylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(6-amino-2′,4′-dioxo-3,4-dihydro-2H,3′H-spiro[naphthalene-1,5′-[1,3]ozazolidin]-3′-yl)-N-[(1S)-1-cyclopropyl-2,2,2-trifluoroethyl]-N-(4-fluorobenzyl)acetamide;
2-[(1R)-5-({[3-(acetylamino)propyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-({[2-(acetylamino)ethyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
tert-butyl [3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)-2,2-dimethylpropyl]carbamate;
N-[2-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)ethyl]propanamide;
2-[(1R)-5-{[(3-amino-2,2-dimethylpropyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-({[3-(acetylamino)phenyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
methyl 2-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]amino}-4,5-dihydro-1H-imidazole-1-carboxylate;
2-{5-bromo-6-[(methylcarbamoyl)amino]-2′,4′-dioxo-3,4-dihydro-2H,3′H-spiro[naphthalene-1,5′-[1,3]ozazolidin]-3′-yl}-N-[(1S)-1-cyclopropyl-2,2,2-trifluoroethyl]-N-(4-fluorobenzyl)acetamide;
2-(5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-{5′-[1-(2-amino-2-oxoethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-2-[(2S)-1,1,1-trifluoropropan-2-yl]acetamide;
N-cyclohexyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-cyclopentyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropyl-2,2,2-trifluoroethyl]-N-(4-fluorobenzyl)-2-{4-hydroxy-6-[(methylcarbamoyl)amino]-2′,4′-dioxo-3,4-dihydro-2H,3′H-spiro[naphthalene-1,5′-[1,3]ozazolidin]-3′-yl}acetamide;
N-benzyl-N-[1-(furan-2-yl)ethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropyl-2,2,2-trifluoroethyl]-N-(4-fluorobenzyl)-2-{4-fluoro-6-[(methylcarbamoyl)amino]-2′,4′-dioxo-3,4-dihydro-2H,3′H-spiro[naphthalene-1,5′-[1,3]ozazolidin]-3′-yl}acetamide;
2-{5′-[1-(2-amino-2-oxoethyl)-1H-pyrazol-4-yl]-3′-hydroxy-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-beta-alanine;
N-cyclopropyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl N-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-beta-alaninate;
N-(3,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(4-fluorobenzyl)-N-(3-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(1S)-5-amino-2′,4′-dioxo-2,3-dihydro-3′H-spiro[indene-1,5′-[1,3]ozazolidin]-3′-yl]-N-[(1R)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
ethyl ({(3R)-3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(3R)-piperidin-3-yl]acetamide;
ethyl ({(3R)-3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]piperidin-1-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(methylsulfonyl)piperidin-4-yl]acetamide;
2-[(4S)-5′-{1-[2-(dimethylamino)-2-oxoethyl]-1H-pyrazol-4-yl}-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4S)-5′-{1-[2-(dimethylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-N-[(2S)-1,1,1-trifluoropropan-2-yl]-2-[(4R)-2,3′,5-trioxo-2′,3′-dihydro-1H-spiro[imidazolidine-4,1′-inden]-1-yl]acetamide;
N-(4-fluorobenzyl)-N-[(2S)-1,1,1-trifluoropropan-2-yl]-2-[(4S)-2,3′,5-trioxo-2′,3′-dihydro-1H-spiro[imidazolidine-4,1′-inden]-1-yl]acetamide;
N-(4-fluorobenzyl)-2-[5′-(1-methyl-1′H-pyrazol-4-yl)-2,3′,5-trioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-{6-fluoro-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(4-fluorobenzyl)-2-[3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(5-amino-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
2-(7′-bromo-1′-methyl-2,5-dioxo-2′,3′-dihydro-1H,1′H-spiro[imidazolidine-4,4′-quinolin]-1-yl)-N-[(1S)-1-cyclopropyl-2,2,2-trifluoroethyl]-N-(4-fluorobenzyl)acetamide;
N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1-phenylethyl)acetamide;
2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)-N-[2-(trifluoromethyl)benzyl]acetamide;
2-(5-bromo-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}-N-[(2S)-1,1,1-trifluoropropan-2-yl]acetamide;
N-(4-fluorobenzyl)-2-[(3′R,4R)-3′-hydroxy-2,5-dioxo-2′,3′-dihydro-1H-spiro[imidazolidine-4,1′-inden]-1-yl]-N-[(2S)-1,1,1-trifluoropropan-2-yl]acetamide;
N-benzyl-N-(cyanomethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-(2-cyanoethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(methylsulfonyl)propan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(1-methyl-1H-pyrazol-4-yl)methyl]acetamide;
N-[(1S)-1-cyclopropylethyl]-N-(4-fluorobenzyl)-2-{4-methoxy-5-[(methylcarbamoyl)amino]-2′,4′-dioxo-2,3-dihydro-3′H-spiro[indene-1,5′-[1,3]ozazolidin]-3′-yl}acetamide;
2-[5-(acetylamino)-4-methoxy-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
N-(4-fluorobenzyl)-2-[(3′S,4R)-3′-fluoro-2,5-dioxo-2′,3′-dihydro-1H-spiro[imidazolidine-4,1′-inden]-1-yl]-N-[(2S)-1,1,1-trifluoropropan-2-yl]acetamide;
2-(7′-bromo-2,2′,5-trioxo-2′,3′-dihydro-1H,1′H-spiro[imidazolidine-4,4′-quinolin]-1-yl)-N-(4-fluorobenzyl)-N-[(2S)-1,1,1-trifluoropropan-2-yl]acetamide;
2-[(1R)-5-({[3-(acetylamino)-2,2-dimethylpropyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-(7′-{1-[2-(methylamino)-2-oxoethyl]-1H-pyrazol-4-yl}-2,2′,5-trioxo-2′,3′-dihydro-1H,1′H-spiro[imidazolidine-4,4′-quinolin]-1-yl)-N-[(2S)-1,1,1-trifluoropropan-2-yl]acetamide;
2-{7′-[1-(2-amino-2-oxoethyl)-1H-pyrazol-4-yl]-2,2′,5-trioxo-2′,3′-dihydro-1H,1′H-spiro[imidazolidine-4,4′-quinolin]-1-yl}-N-(4-fluorobenzyl)-N-[(2S)-1,1,1-trifluoropropan-2-yl]acetamide;
N-cyclopentyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-[(1S)-1-cyclopropyl-2,2,2-trifluoroethyl]-N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}acetamide;
2-(5′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2,2,2-trifluoro-N-[1-(2-{(4-fluorobenzyl)[(2S)-1,1,1-trifluoropropan-2-yl]amino}-2-oxoethyl)-1′-methyl-2,5-dioxo-2′,3′-dihydro-1′H-spiro[imidazolidine-4,4′-quinolin]-7′-yl]acetamide;
N-[1-(2-{[(1S)-1-cyclopropylethyl](4-fluorobenzyl)amino}-2-oxoethyl)-1′-methyl-2,5-dioxo-2′,3′-dihydro-1′H-spiro[imidazolidine-4,4′-quinolin]-7′-yl]-2,2,2-trifluoroacetamide;
N-(4-fluorobenzyl)-2-[(3′S,4S)-3′-hydroxy-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(pyridin-3-yl)acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1-methyl-1′H-pyrazol-4-yl)acetamide;
N-(4-fluorobenzyl)-2-[3′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-benzyl-N-(2,2-difluorocyclopentyl)-2-{5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-(2-methylcyclopropyl)acetamide;
N-(2,2-dimethylcyclopentyl)-N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-(2-methylcyclopentyl)acetamide;
2-(5′-bromo-3′-hydroxy-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-(5-{[(2-cyanoethyl)carbamoyl]amino}-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
N-(4-fluorobenzyl)-2-[(3′R,4S)-3′-fluoro-2,5-dioxo-2′,3′-dihydro-1H-spiro[imidazolidine-4,1′-inden]-1-yl]-N-[(2S)-1,1,1-trifluoropropan-2-yl]acetamide;
methyl [(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamate;
4-({[(1R)-3′-(2-{(4-fluorobenzyl)[(2S)-1,1,1-trifluoropropan-2′-yl]amino}-2-oxoethyl)-2′,4′-dioxo-2,3-dihydrospiro[indene-1,5′-[1,3]ozazolidin]-5-yl]carbamoyl}amino)butanoic acid;
N²-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3′]ozazolidin]-5-yl]-2-methylalaninamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylalaninamide;
N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
N-cyclopentyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-phenylethyl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-oxazol-4-ylmethyl)acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-[1-(pyridin-2-yl)ethyl]acetamide;
N-(4-fluorobenzyl)-2-{5′-[(methylcarbamoyl)amino]-2,3′,5-trioxo-2′,3′-dihydro-1H-spiro[imidazolidine-4,1-inden]-1-yl}-N-[(2S)-1,1,1-trifluoropropan-2-yl]acetamide;
N-(4-chlorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3,5-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-(5-{[(cyanomethyl)carbamoyl]amino}-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
2-[(1R)-5-{[(cyanomethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclohexyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-phenylethyl]acetamide;
2-(5′-amino-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-pyrrolidin-3-yl]acetamide;
N-(4-fluorobenzyl)-N-(trans-3-hydroxycyclobutyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-1-(methylsulfonyl)pyrrolidin-3-yl]acetamide;
ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidin-1-yl}sulfonyl)carbamate;
N-(4-fluorobenzyl)-2-[6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide;
2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{3′-hydroxy-5′-[1-(2-methylpropyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
N-(1-cyanopropan-2-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(4S)-5′-bromo-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-(5′-bromo-3′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
5-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)pentanoic acid;
N-[(1S)-1-cyclopropyl-2,2,2-trifluoroethyl]-N-(4-fluorobenzyl)-2-[4-fluoro-5-(1-methyl-1H-pyrazol-4-yl)-2′,4′-dioxo-2,3-dihydro-3′H-spiro[indene-1,5′-[1,3]ozazolidin]-3′-yl]acetamide;
N-(4-fluorobenzyl)-2-[6-fluoro-5-(1-methyl-1H-pyrazol-4-yl)-2′,4′-dioxo-2,3-dihydro-3′H-spiro[indene-1,5′-[1,3]ozazolidin]-3′-yl]-N-(2S)-1,1,1-trifluoropropan-2-yl]acetamide;
2-{(4S)-5′-[1-(2-amino-2-oxoethyl)-1H-pyrazol-4-yl]-2,5-dioxo-2′,3′-dihydro-1H-spiro[imidazolidine-4,3′-inden]-1-yl}-N-(4-fluorobenzyl)-N-[(2S)-1,1,1-trifluoropropan-2-yl]acetamide;
N-[(1S)-1-cyclopropylethyl]-N-(4-fluorobenzyl)-2-[1′-methyl-7′-(1-methyl-1H-pyrazol-4-yl)-2,5-dioxo-2′,3′-dihydro-1H,1′H-spiro[imidazolidine-4,4′-quinolin]-1-yl]acetamide;
N-(4-fluorobenzyl)-2-[7′-(3-methyl-1H-pyrazol-4-yl)-2,2′,5-trioxo-2′,3′-dihydro-1H,1′H-spiro[imidazolidine-4,4′-quinolin]-1-yl]-N-[(2S)-1,1,1-trifluoropropan-2-yl]acetamide;
N-(4-fluorobenzyl)-2-[(4S)-5′-(1-methyl-1H-pyrazol-4-yl)-2,3′,5-trioxo-2′,3′-dihydro-1H-spiro[imidazolidine-4,1′-inden]-1-yl]-N-[(2S)-1,1,1-trifluoropropan-2-yl]acetamide;
N-[(3R)-1-acetylpyrrolidin-3-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-1-(2-sulfamoylethyl)pyrrolidin-3-yl]acetamide;
N-(4-fluorobenzyl)-N-(trans-4-hydroxycyclohexyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
methyl (1R,3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
N-(4-fluorobenzyl)-2-[(3′R,4S)-3′-hydroxy-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(3′S,4S)-3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(4S)-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl]-N-[(2S)-1,1,1-trifluoropropan-2-yl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-3-(methylsulfonyl)tetrahydropyrimidine-1(2H)-carboxamide;
N-(4-fluorobenzyl)-2-[(3′S,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(4S)-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(3′S,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(3′R,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
N²-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]glycinamide;
2-{(1R)-5-[(methylcarbamoyl)amino]-2′,4′-dioxo-2,3-dihydro-3′H-spiro[indene-1,5′-[1,3]ozazolidin]-3′-yl}-N-[1-(2-methylphenyl)ethyl]-N-(2-methylpropyl)acetamide;
N-(2-chlorobenzyl)-N-(cyclopropylmethyl)-2-{(1R)-5-[(methylcarbamoyl)amino]-2′,4′-dioxo-2,3-dihydro-3′H-spiro[indene-1,5′]-[1,3]ozazolidin]-3′-yl}acetamide;
N-(2-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)acetamide;
2-[(3′S,4S)-5′-bromo-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-N-(4-methoxyphenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(4-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-[(2-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-[(3′R,4S)-3′-fluoro-5′-{1-[2-(methylamino)-2-oxoethyl]-]H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(3′R,4S)-5′-bromo-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{(4S)-5′-[1-(2-amino-2-oxoethyl)-1H-pyrazol-4-yl]-2,3′,5-trioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(trifluoromethyl)cyclopropyl]acetamide;
2-[(1R)-5-(3,4-dihydro-2H-pyrrol-5-ylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(3,4-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(2,5-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3-chlorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(3′S,4S)-5′-[1-(2-amino-2-oxoethyl)-1H-pyrazol-4-yl]-3′-hydroxy-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-{5-[1-(2-amino-2-oxoethyl)-1H-pyrazol-4-yl]-6-fluoro-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(3R)-pyrrolidin-3-yl]acetamide;
ethyl ({(3R)-3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]pyrrolidin-1-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(piperidin-4-yl)acetamide;
ethyl ({4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
2-[(3′S,4S)-5′-{1-[2-(dimethylamino)-2-oxoethyl]-1H-pyrazol-4-yl}-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]azetidine-1-carboxylate;
N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}-N-[2-(trifluoromethyl)benzyl]acetamide;
N-(2-chloro-4-fluorobenzyl)-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(3′R,4S)-5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-N-(3-methoxyphenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-(pyridin-4-yl)acetamide;
N-(4-fluorobenzyl)-2-[(4S)-5′-{1-[2-(methylamino)-2-oxoethyl]-1H-pyrazol-4-yl}-2,5-dioxo-1H-spiro[imidazolidine-4,1′-inden]-1-yl]-N-[(2S)-1,1,1-trifluoropropan-2-yl]acetamide;
N-(3,4-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(2,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-[(1S)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-[(1S)-1-cyclopropyl-2,2,2-trifluoroethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-[(1R)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-benzyl-N-[(1S)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-benzyl-N-[(1R)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide;
2-(4′-bromo-2,5-dioxo-2′,3′-dihydro-1′H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[4′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-(6-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl)-N-[(2S)-1,1,1-trifluoropropan-2-yl]acetamide;
2-[(3′S,4S)-5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
methyl (1S,3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(tetrahydro-2H-pyran-4-yl)acetamide;
N-(1,1-dioxidotetrahydro-2H-thiopyran-4-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2,2-difluorocyclopentyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-cyclohexyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
2-[(3S)-6-amino-1,1-dioxido-2′,5′-dioxo-1′H-spiro[1-benzothiophene-3,4′-imidazolidin]-1′-yl]-N-(4-fluorobenzyl)-N-[(2S)-1,1,1-trifluoropropan-2-yl]acetamide;
2-[(3′R,4S)-5′-bromo-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-(5′-bromo-6′-fluoro-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-(5′-bromo-6′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-(6′-fluoro-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-(6′-fluoro-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-[4′-(1-methyl-1,2,3,6-tetrahydropyridin-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-[(1S)-1-cyclopropyl-2,2,2-trifluoroethyl]-N-(4-fluorobenzyl)-2-[7-(1-methyl-1H-pyrazol-4-yl)-2′,4′-dioxo-1H,3′H-spiro[isochromene-4,5′-[1,3]ozazolidin]-3′-yl]acetamide;
2-(7-amino-2′,4′-dioxo-1H,3′H-spiro[isochromene-4,5′-[1,3]ozazolidin]-3′-yl)-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{7-[(methylcarbamoyl)amino]-2′,4′-dioxo-1H,3′H-spiro[isochromene-4,5′-[1,3]ozazolidin]-3′-yl}acetamide;
N-(4-fluorobenzyl)-2-{7-[(methylcarbamoyl)amino]-2′,4′-dioxo-1H,3′H-spiro[isochromene-4,5′-[1,3]ozazolidin]-3′-yl}-N-[(2S)-1,1,1-trifluoropropan-2-yl]acetamide;
N-(4-fluorobenzyl)-2-[7-(1-methyl-1H-pyrazol-4-yl)-2′,4′-dioxo-1H,3′H-spiro[isochromene-4,5′-[1,3]ozazolidin]-3′-yl]-N-[(2S)-1,1,1-trifluoropropan-2-yl]acetamide;
N-(4-fluorobenzyl)-2-(7-{1-[2-(methylamino)-2-oxoethyl]-1H-pyrazol-4-yl}-2′,4′-dioxo-1H,3′H-spiro[isochromene-4,5′-[1,3]ozazolidin]-3′-yl)-N-[(2S)-1,1,1-trifluoropropan-2-yl]acetamide;
N-(4-fluorobenzyl)-2-[(3′R,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(3R)-6-amino-1,1-dioxido-2′,5′-dioxo-1′H-spiro[1-benzothiophene-3,4′-imidazolidin]-1′-yl]-N-(4-fluorobenzyl)-N-[(2S)-1,1,1-trifluoropropan-2-yl]acetamide;
N-(4-fluorobenzyl)-N-{1-[2-(methylamino)-2-oxoethyl]-1H-pyrazol-4-yl}-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[1-(cyanomethyl)-1H-pyrazol-4-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl 3-{1-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]ethyl}azetidine-1-carboxylate;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylic acid;
ethyl ({(3S)-3-[(4-fluorobenzyl) {[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
ethyl ({(3S)-3-[{[(1R)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}(4-fluorobenzyl)amino]pyrrolidin-1-yl}sulfonyl)carbamate;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}acetate;
ethyl ({(3R)-3-(4-fluorobenzyl) {[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]piperidin-1-yl}sulfonyl)carbamate;
ethyl ({(3R)-3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]piperidin-1-yl}sulfonyl)carbamate;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}acetic acid;
2-{2,5-dioxo-5′-[(2,2,2-trifluoroethyl)amino]-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-[(4S)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4R)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{(3′R,4S)-5′-[1-(2-amino-2-oxoethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-(5′-bromo-6′-fluoro-3′-hydroxy-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-(6′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2 S)-1,1,1,1-trifluoropropan-2-yl]acetamide;
2-{5′-[1-(2-amino-2-oxoethyl)-1H-pyrazol-4-yl]-6′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-{6′-fluoro-5′-[1-(2-hydroxyethyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-[6′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(5′-bromo-3′,6′-difluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
tert-butyl 4-[1-(2-{(4-fluorobenzyl)[(2S)-1,1,1-trifluoropropan-2-yl]amino}-2-oxoethyl)-2,5-dioxo-2′,3′-dihydrospiro[imidazolidine-4,1′-inden]-4′-yl]-3,6-dihydropyridine-[(2H)-carboxylate;
N-(4-fluorobenzyl)-2-{(4S)-7-[(methylcarbamoyl)amino]-2′,4′-dioxo-1H,3′H-spiro[isochromene-4,5′-[1,3]ozazolidin]-3′-yl}-N-[(2S)-1,1,1-trifluoropropan-2-yl]acetamide;
2-[2,5-dioxo-4′-(1,2,3,6-tetrahydropyridin-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{(4R)-7-[(methylcarbamoyl)amino]-2′,4′-dioxo-1H,3′H-spiro[isochromene-4,5′-[1,3]ozazolidin]-3′-yl}-N-[(2S)-1,1,1-trifluoropropan-2-yl]acetamide;
2-{5′-[1-(2-amino-2-oxoethyl)-1H-pyrazol-4-yl]-3′,6′-difluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-{4-[3′,6′-difluoro-1-(2-{(4-fluorobenzyl) [(2S)-1,1,1-trifluoropropan-2-yl]amino}-2-oxoethyl)-2,5-dioxo-2′,3′-dihydro-1H-spiro[imidazolidine-4,1′-inden]-5′-yl]-1H-pyrazol-1-yl}-N,N-dimethylacetamide;
2-[3′,6′-difluoro-5′-(1-methyl-1H-pyrazol-4-yl)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{3′,6′-difluoro-5′-[1-(2-hydroxyethyl)-1H-pyrazol-4-yl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-[(4R)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
tert-butyl (3 S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-piperidin-3-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3 S)-piperidin-3-yl]acetamide;
N-[4-amino-3-(hydroxymethyl)butan-2-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({(3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzamide;
tert-butyl 2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(pyrrolidin-2-ylmethyl)acetamide;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexylidene}acetate;
N-ethyl-4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxamide;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexylidene}acetic acid;
2-[(4S)-2,5-dioxo-5′-(1H-pyrazol-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{5′-[(1-methyl-1H-pyrazol-3-yl)amino]-2,5-dioxo-2′,3′-dihydro-1H-spiro[imidazolidine-4,1′-inden]-1-yl}-N-[(2S)-1,1,1-trifluoropropan-2-yl]acetamide;
2-[(4S)-5′-amino-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{4′-[(dimethylamino)methyl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-[4′-(morpholin-4-ylmethyl)-2,5-dioxo-2′,3′-dihydro-1H-spiro[imidazolidine-4,1′-inden]-1-yl]-N-[(2S)-1,1,1-trifluoropropan-2-yl]acetamide;
2-[2,5-dioxo-4′-(pyrrolidin-1-ylmethyl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-(4′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[(1R)-5-(oxetan-3-ylamino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzamide;
N-[(1-acetylpyrrolidin-2-yl)methyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{[1-(methylsulfonyl)pyrrolidin-2-yl]methyl}acetamide;
2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}-N-methylpyrrolidine-1-carboxamide;
N-(3,4-difluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{[1-(methylsulfamoyl)pyrrolidin-2-yl]methyl}acetamide;
N-(cyclopropylmethyl)-N-(2,3-dihydro-1H-inden-1-yl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
methyl trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylate;
trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylic acid;
trans-4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]cyclohexanecarboxylic acid;
2-[(4R)-7′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
trans-4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}cyclohexanecarboxylic acid;
2-[(4S)-7′-bromo-2,5-dioxo-2′,3′-dihydro-H-spiro[imidazolidinne-4,1′-inden]-1-yl]-N-(4-fluorobenzyl)-N-[(2S)-1,1,1-trifluoropropan-2-yl]acetamide;
ethyl ({4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]piperidin-1-yl}sulfonyl) carbamate;
ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexyl}acetic acid;
2-[(4S)-5′-{[(2-cyanoethyl)carbamoyl]amino}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{(4S)-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(4S)-5′-[(methylcarbamoyl)amino]-2,3′,5-trioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(3′S,4S)-3′-hydroxy-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(3′R,4S)-3′-fluoro-5′-[1-(2-hydroxyethyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-3-methylazetidin-1-yl}sulfonyl)carbamate;
2-[4′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4R)-5′-bromo-6′-fluoro-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(3-cyanophenyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(3′R,4S)-3′-fluoro-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
N-(3,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(4-fluorobenzyl)-2-{7′-[(methylcarbamoyl)amino]-2,2′,5-trioxo-2′,3′-dihydro-1H, 1′H-spiro[imidazolidine-4,4′-quinolin]-1-yl}-N-[(2S)-1,1,1-trifluoropropan-2-yl]acetamide;
tert-butyl (4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetate;
(4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetic acid;
{4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
(4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}cyclohexylidene)acetic acid;
tert-butyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}carbamate;
N-(3-aminocyclobutyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[3-(acetylamino)cyclobutyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{3-[(methylsulfonyl)amino]cyclobutyl}acetamide;
N-(4-fluorobenzyl)-2-[(3′R,4S)-3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-[(4R)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(3′E,4S)-3′-(hydroxyimino)-5′-(1-methyl-1H-pyrazol-4-yl)-2,5-dioxo-2′,3′-dihydro-1H-spiro[imidazolidine-4,1′-inden]-1-yl]-N-[(2 S)-1,1,1-trifluoropropan-2-yl]acetamide;
2,2′-({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}imino)diacetic acid;
N-{3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}glycine;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxylate;
methyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}carbamate;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}sulfamoyl)carbamate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxylic acid;
2-[(3′R,4S)-3′,6′-difluoro-5′-{1-[2-(methylamino)-2-oxoethyl]-1H-pyrazol-4-yl}-2,5-dioxo-2′,3′-dihydro-1H-spiro[imidazolidine-4, 1′-inden]-1-yl]-N-(4-fluorobenzyl)-N-[(2S)-1,1,1-trifluoropropan-2-yl]acetamide;
2-{4-[(3′R,4S)-3′,6′-difluoro-1-(2-{(4-fluorobenzyl) [(2S)-1,1,1-trifluoropropan-2-yl]amino}-2-oxoethyl)-2,5-dioxo-2′,3′-dihydro-1H-spiro[imidazolidine-4,1′-inden]-5′-yl]-1H-pyrazol-1-yl}-N,N-dimethylacetamide;
2-[(3′S,4R)-3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{4-[(3′S,4R)-3′,6′-difluoro-1-(2-{(4-fluorobenzyl)[(2S)-1,1,1-trifluoropropan-2-yl]amino}-2-oxoethyl)-2,5-dioxo-2′,3′-dihydro-1H-spiro[imidazolidine-4,1′-inden]-5′-yl]-1H-pyrazol-1-yl}-N,N-dimethylacetamide;
ethyl [(4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}piperidin-1-yl)sulfonyl]carbamate;
ethyl ({4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]piperidin-1-yl}sulfonyl)carbamate;
2-[(1 S,3S)-5-bromo-3-hydroxy-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(5-chloro-4-cyano-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
benzyl 3-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}azetidine-1-carboxylate;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoate;
tert-butyl 4-[{[(4S)-5′-bromo-2,5-dioxo-2′,3′-dihydro-1H-spiro[imidazolidine-4,1′-inden]-1-yl]acetyl}(4-fluorobenzyl)amino]piperidine-1-carboxylate;
N-(4-fluorobenzyl)-2-[(1 S,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
ethyl ({4-[{[(4S)-5′-amino-2,5-dioxo-2′,3′-dihydro-1H-spiro[imidazolidine-4,1′-inden]-1-yl]acetyl}(4-fluorobenzyl)amino]piperidin-1-yl}sulfonyl)carbamate;
ethyl ({3,3-difluoro-4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]pyrrolidin-1-yl}sulfonyl)carbamate;
ethyl ({3,3-difluoro-4-[(4-fluorobenzyl) {[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]acetyl}amino]pyrrolidin-1-yl}sulfonyl)carbamate;
ethyl ({3,3-difluoro-4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
ethyl ({3,3-difluoro-4-[(4-fluorobenzyl){[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
N-(4-fluorobenzyl)-2-[(1S)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1S)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-N-(3-methoxycyclobutyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-N-methylcyclobutanecarboxamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxamide;
2-{(3′R,4S)-5′-[1-(difluoromethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-(5-amino-4-cyano-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
2-[2,5-dioxo-4′-(piperidin-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetic acid;
(2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidin-1-yl)acetic acid;
methyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetate;
N-(4-fluorobenzyl)-2-[(4S)-7′-hydroxy-2,5-dioxo-2′,3′-dihydro-1H-spiro[imidazolidine-4,1′-inden]-1-yl]-N-[(2S)-1,1,1-trifluoropropan-2-yl]acetamide;
{3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetic acid;
tert-butyl {4-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]cyclohexylidene}acetate;
tert-butyl {4-[{[(4S)-5′-bromo-2,5-dioxo-2′,3′-dihydro-1H-spiro[imidazolidine-4,1′-inden]-1-yl]acetyl}(4-fluorobenzyl)amino]cyclohexylidene}acetate;
tert-butyl {4-[(4-fluorobenzyl) {[(1S)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetate;
tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate;
{4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetic acid;
{4-[{[(4S)-5′-amino-2,5-dioxo-2′,3′-dihydro-1H-spiro[imidazolidine-4,1′-inden]-1-yl]acetyl}(4-fluorobenzyl)amino]cyclohexylidene}acetic acid;
N-(4-fluorobenzyl)-2-[(1 S,3R)-3-fluoro-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(4S)-5′-bromo-6′-fluoro-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3′R,4R)-5′-bromo-6′-fluoro-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3′S,4S)-5′-bromo-6′-fluoro-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]-N-(tetrahydro-2H-pyran-4-yl)acetamide;
2-(2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-{6′-fluoro-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
2-{(3′R,4S)-3′,6′-difluoro-5′-[(methylcarbamoyl)amino]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(azetidin-3-ylmethyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
{4-[{[(1R)-2′,4′-dioxo-2,3-dihydro-3′H-spiro[indene-1,5′-[1,3]ozazolidin]-3′-yl]acetyl}(4-fluorobenzyl)amino]cyclohexylidene}acetic acid;
methyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate;
{4-[(4-fluorobenzyl) {[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
tert-butyl {trans-4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexyl}carbamate;
N-(trans-4-aminocyclohexyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{trans-4-[(methylsulfonyl)amino]cyclohexyl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(trans-4-{[(trifluoromethyl)sulfonyl]amino}cyclohexyl)acetamide;
N-(4-fluorobenzyl)-2-[(4R)-7′-hydroxy-2,5-dioxo-2′,3′-dihydro-1H-spiro[imidazolidine-4,1′-inden]-1-yl]-N-[(2S)-1,1,1-trifluoropropan-2-yl]acetamide;
tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetate;
{4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetic acid;
tert-butyl 6-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-2-azaspiro[3.3]heptane-2-carboxylate;
N-(2-azaspiro[3.3]hept-6-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(1 S,3R)-5-bromo-3-hydroxy-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1-acetylazetidin-3-yl)methyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-[(2S)-1,1,1-trifluoropropan-2-yl]-2-(2′,3,4′-trioxo-1,2,3,4, 8,9-hexahydro-3′H-spiro[cyclopenta[f]quinazoline-7,5′-[1,3]ozazolidin]-3′-yl)acetamide;
ethyl [(3-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}azetidin-1-yl)sulfonyl]carbamate;
ethyl [(2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidin-1-yl)sulfonyl]carbamate;
2-[(4S)-2,5-dioxo-5′-(1H-pyrazol-5-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
ethyl ({6-[(4-fluorobenzyl)({(1R)-5-[(methylcarbamycarbamoyl)amino]-2′,4′-dioxo-2,3-dihydro-3′H-spiro[indene-1,5′-[1,3]ozazolidin]-3′-yl}acetyl)amino]-2-azaspiro[3.3]hept-2-yl}sulfonyl)carbamate;
4-[(4-fluorobenzyl) {[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexanecarboxylic acid;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(3-{[(trifluoromethyl)sulfonyl]amino}cyclobutyl)acetamide;
tert-butyl 3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]benzoate;
tert-butyl 3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]benzoate;
3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]benzoic acid;
2-[(4S)-5′-{1-[2-(dimethylamino)ethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
tert-butyl 4-({4-[(4S)-1-(2-{(4-fluorobenzyl)[(2S)-1,1,1-trifluoropropan-2-yl]amino}-2-oxoethyl)-2,5-dioxo-2′,3′-dihydrospiro[imidazolidine-4,1′-inden]-5′-yl]-1H-pyrazol-1-yl}methyl)piperidine-1-carboxylate;
2-{(4S)-2,5-dioxo-5′-[1-(piperidin-4-ylmethyl)-1H-pyrazol-4-yl]-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-{6-[(1-methyl-1H-pyrazol-4-yl)amino]-1,1-dioxido-2′,5′-dioxo-1′H-spiro[1-benzothiophene-3,4′-imidazolidin]-1′-yl}-N-[(2S)-1,1,1-trifluoropropan-2-yl]acetamide;
N-(4-fluorobenzyl)-2-[(1S)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(azetidin-3-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]azetidin-1-yl}sulfonyl)carbamate;
2-[(1S)-5-bromo-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-(6-methoxy-1,1-dioxido-2′,5′-dioxo-1′H-spiro[1-benzothiophene-3,4′-imidazolidin]-1′-yl)-N-[(2S)-1,1,1-trifluoropropan-2-yl]acetamide;
N-(4-fluorobenzyl)-2-[(3 S)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide;
N-(4-fluorobenzyl)-2-[(3R)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide;
N-(4-fluorobenzyl)-2-[(1 S,3R)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-benzyl-N-(1-cyclopropylethyl)-2-(2,5-dioxo-2′,3′-dihydro-1H-spiro[imidazolidine-4,1′-inden]-1-yl)acetamide;
N-(1-cyclopropylethyl)-2-(2,5-dioxo-2′,3′-dihydro-1H-spiro[imidazolidine-4,1′-inden]-1-yl)-N-(1H-imidazol-4-ylmethyl)acetamide;
N-[(1S)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-D-valinamide;
N-benzyl-N-(1-cyclopropylethyl)-2-(2,2-dimethyl-2′,5′-dioxo-1′H-spiro[1-benzofuran-3,4′-imidazolidin]-1′-yl)acetamide;
N-benzyl-N-(1-cyclopropylethyl)-2-(1′,3-dimethyl-2,2′,5-trioxo-1′,2′-dihydro-1H-spiro[imidazolidine-4,3′-indol]-1-yl)acetamide;
N-benzyl-N-(1-cyclopropylethyl)-2-(2,5-dioxo-2′,3′-dihydro-1H-spiro[imidazolidine-4,1′-inden]-1-yl)-2-methylpropanamide;
N-benzyl-N-(1-cyclopropylethyl)-2-[2,5-dioxo-3-(prop-2-en-1-yl)-2′,3′-dihydro-1H-spiro[imidazolidine-4,1′-inden]-1-yl]acetamide;
N-(1-cyclopropylethyl)-2-(2,5-dioxo-2′,3′-dihydro-1H-spiro[imidazolidine-4,1′-inden]-1-yl)-N-(2,2,2-trifluoroethyl)acetamide;
N-(1-cyclopropylethyl)-2-(2,5-dioxo-2′,3′-dihydro-1H-spiro[imidazolidine-4,1′-inden]-1-yl)-N-[3-(trifluoromethoxy)benzyl]acetamide;
N-(but-2-yn-1-yl)-N-(1-cyclopropylethyl)-2-(2,5-dioxo-2′,3′-dihydro-1H-spiro[imidazolidine-4,1′-inden]-1-yl)acetamide;
N-(1-cyclopropylethyl)-N-{[6-(difluoromethoxy)naphthalen-2-yl]methyl}-2-(2,5-dioxo-2′,3′-dihydro-1H-spiro[imidazolidine-4,1′-inden]-1-yl)acetamide;
N-benzyl-N-(1-cyclopropylethyl)-2-[5′-(methylsulfonyl)-2,5-dioxo-2′,3′-dihydro-1H-spiro[imidazolidine-4,1′-inden]-1-yl]acetamide;
N-benzyl-N-(1-cyclobutylethyl)-2-(7′-methoxy-2,5-dioxo-2′,3′-dihydro-1H-spiro[imidazolidine-4,1′-inden]-1-yl)acetamide;
N-benzyl-N-(1-cyclopropylethyl)-2-(1-methyl-2′,5′-dioxo-5,6-dihydro-1H,1′H-spiro[cyclopenta[c]pyrazole-4,4′-imidazolidin]-1′-yl)acetamide;
N-benzyl-N-(1-cyclopropylethyl)-2-[5′-(hydroxymethyl)-3-methyl-2,5-dioxo-2′,3′-dihydro-1H-spiro[imidazolidine-4,1′-inden]-1-yl]acetamide;
N-[(1S)-1-cyclobutylethyl]-2-[(4R)-2,5-dioxo-2′,3′-dihydro-1H-spiro[imidazolidine-4,1′-inden]-1-yl]-N-(prop-2-yn-1-yl)acetamide;
N³-[(1S)-1-cyclobutylethyl]-N³-{[(4R)-2,5-dioxo-2′,3′-dihydro-1H-spiro[imidazolidine-4,1′-inden]-1-yl]acetyl}-beta-alaninamide;
N-(biphenyl-4-ylmethyl)-N-[(1S)-1-cyclobutylethyl]-2-[(4R)-2,5-dioxo-2′,3′-dihydro-1H-spiro[imidazolidine-4,1′-inden]-1-yl]acetamide;
N²-[(1S)-1-cyclobutylethyl]-N²-{[(4R)-2,5-dioxo-2′,3′-dihydro-1H-spiro[imidazolidine-4,1′-inden]-1-yl]acetyl}glycinamide;
N-[(1S)-1-cyclobutylethyl]-2-[(4R)-2,5-dioxo-2′,3′-dihydro-1H-spiro[imidazolidine-4,1′-inden]-1-yl]-N-[(1-methyl-2-oxo-1,2-dihydropyridin-4-yl)methyl]acetamide;
N-benzyl-N-[(1R)-1-cyclopropylethyl]-2-[(4R)-2,5-dioxo-2′,3′-dihydro-1H-spiro[imidazolidine-4,1′-inden]-1-yl]acetamide;
N-benzyl-N-[(1S)-1-cyclopropylethyl]-2-[(4S)-2,5-dioxo-2′,3′-dihydro-1H-spiro[imidazolidine-4,1′-inden]-1-yl]acetamide;
N-benzyl-N-[(1S)-1-cyclopropylethyl]-2-[(4R)-2,5-dioxo-2′,3′-dihydro-1H-spiro[imidazolidine-4,1′-inden]-1-yl]acetamide;
N-benzyl-N-[(1R)-1-cyclopropylethyl]-2-[(4S)-2,5-dioxo-2′,3′-dihydro-1H-spiro[imidazolidine-4,1′-inden]-1-yl]acetamide;
N-[4-(acetylamino)benzyl]-N-[(1S)-1-cyclobutylethyl]-2-[(4R)-2,5-dioxo-2′,3′-dihydro-1H-spiro[imidazolidine-4,1′-inden]-1-yl]acetamide;
N-[(1S)-1-cyclobutylethyl]-2-[(4R)-2,5-dioxo-2′,3′-dihydro-1H-spiro[imidazolidine-4,1′-inden]-1-yl]-N-(4-ethynylbenzyl)acetamide;
N-[(1S)-1-cyclobutylethyl]-2-[(4R)-2,5-dioxo-2′,3′-dihydro-1H-spiro[imidazolidine-4, 1′-inden]-1-yl]-N-({1-[2-(methylamino)-2-oxoethyl]-1H-1,2,3-triazol-4-yl}methyl)acetamide;
2-{4-[([(1S)-1-cyclobutylethyl]{[(4R)-2,5-dioxo-2′,3′-dihydro-1H-spiro[imidazolidine-4,1′-inden]-1-yl]acetyl}amino)methyl]-1H-1,2,3-triazol-1-yl}-N,N-dimethylacetamide;
N³-[(1S)-1-cyclobutylethyl]-N³-{[(4R)-2,5-dioxo-2′,3′-dihydro-1H-spiro[imidazolidine-4,1′-inden]-1-yl]acetyl}-N-methyl-beta-alaninamide;
N-benzyl-N-(1-cyclopropylethyl)-2-[5′-(6-hydroxypyridin-3-yl)-2,5-dioxo-2′,3′-dihydro-1H-spiro[imidazolidine-4,1′-inden]-1-yl]acetamide;
N-benzyl-N-[(1S)-1-cyclopropylethyl]-2-[5′-(formylamino)-2,5-dioxo-2′,3′-dihydro-1H-spiro[imidazolidine-4,1′-inden]-1-yl]acetamide;
N-benzyl-N-[(1S)-1-cyclopropylethyl]-2-[2′,4′-dioxo-5-(2H-tetrazol-5-yl)-2,3-dihydro-3′H-spiro[indene-1,5′-[1,3]ozazolidin]-3′-yl]acetamide;
2,2′-({[3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]carbonyl}imino)diacetic acid;
N-benzyl-2-[(4R)-5′-bromo-2,5-dioxo-2′,3′-dihydro-1H-spiro[imidazolidine-4,1′-inden]-1-yl]-N-[(1S)-1-cyclopropylethyl]acetamide;
N-(4-fluorobenzyl)-2-[(1 S,3R)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1 S,3R)-5-[1-(2-amino-2-oxoethyl)-1H-pyrazol-4-yl]-3-hydroxy-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1S)-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[1,1-dioxido-2′,5′-dioxo-6-(pyridin-3-yl)-1H-spiro[1-benzothiophene-3,4′-imidazolidin]-1′-yl]-N-(4-fluorobenzyl)-N-[(2S)-1,1,1-trifluoropropan-2-yl]acetamide;
2-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
methyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanoate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanoic acid;
2-{(1S)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(3′S,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(1R,3R)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R,3R)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1 S,3S)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1S)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1 S,3R)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyridin-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(pyridin-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(pyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(6-oxo-1,6-dihydropyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(6-oxo-1,6-dihydropyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-phenyl-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-phenyl-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclohexyl-N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]acetamide;
N-(4-fluorobenzyl)-2-[(1R)-5-(1-methyl-1H-pyrazol-4-yl)-2′,4′-dioxo-2,3-dihydro-3′H-spiro[indene-,5′-[1,3]ozazolidin]-3′-yl]-N-(tetrahydro-2H-pyran-4-yl)acetamide;
3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclobutanecarboxylic acid;
4-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexanecarboxylic acid;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-{trans-4-[(methylsulfonyl)amino]cyclohexyl}acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-(3-{[(trifluoromethyl)sulfonyl]amino}cyclobutyl)acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-[1-(methylsulfonyl)piperidin-4-yl]acetamide;
2-[(1R,3S)-5-bromo-3-hydroxy-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R,3S)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2,2′-({3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclobutyl}imino)diacetic acid;
N-(4-fluorobenzyl)-2-[(1 S,3R)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(1,2-oxazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1S)-5-(1,2-oxazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1 S,3R)-3-fluoro-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
(4-{[(5′-bromo-6′-fluoro-2,5-dioxo-2′,3′-dihydro-1H-spiro[imidazolidine-4,1′-inden]-1-yl)acetyl](4-fluorobenzyl)amino}cyclohexylidene)acetic acid;
{4-[(4-fluorobenzyl) {[6′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
tert-butyl (4-{[(5′-bromo-6′-fluoro-2,5-dioxo-2′,3′-dihydro-1H-spiro[imidazolidine-4,1′-inden]-1-yl)acetyl](4-fluorobenzyl)amino}cyclohexylidene)acetate;
2-[(1R,3S)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R,3S)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
{4-[(4-fluorobenzyl) {[(1 S,3R)-3-fluoro-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetic acid;
N-(4-fluorobenzyl)-2-[(1R,3R)-3-fluoro-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R,3R)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide; and pharmaceutically acceptable salts thereof.

In another aspect, the present invention relates to compounds of Formula (Xa) or (Xb)

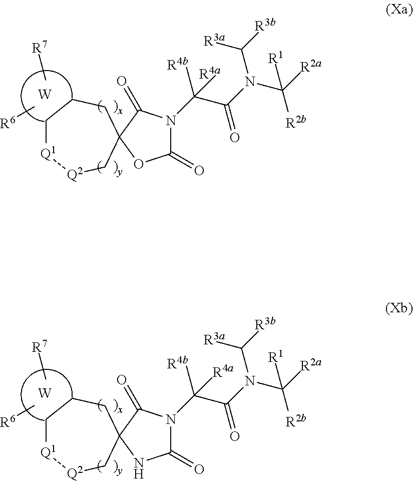

or a stereoisomer, tautomer or pharmaceutically acceptable salt thereof,
wherein
[symbol] is —C(R¹⁰)₂—C(R¹⁴)₂—, —O—C(R¹⁴)₂—, —O—C(O)—, —S(O)₂—C(R¹⁴)₂—, —S—C(R¹⁴)₂—, —NR⁹—C(O)—, —NR⁹—C(R¹⁴)₂—, —C(R¹⁰)₂—O—, —C(R¹⁰)₂—, or —C(R¹⁰)═C(R¹⁴)—;
W is arylene or heteroarylene;
R¹ is carbocyclyl or heterocyclyl;
R^{2a} and R^{2b} are each independently H, D, or C₁-C₆ alkyl;
R^{3a} is hydrogen, C(O)NH₂, C₁-C₆ alkyl, aryl, cycloalkyl or heterocyclyl; and
R^{3b} is C₁-C₆ alkyl, aryl, cycloalkyl or heterocyclyl; or
R^{3a} and R^{3b} taken together with the carbon to which they are attached form an arene, cycloalkane, or heterocycle;
R^{4a} and R^{4b} are each independently H, D or C₁-C₆ alkyl;
R⁶ and R⁷ are each independently H, halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═S)N(R¹²)₂, —NHC(═N—C≡N)NR¹², —NHC(═N—C≡N)SR¹², or —NHS(O)ₘR¹²;
R⁹ is independently H or C₁-C₆ alkyl;
R¹⁰, at each occurrence, is independently H, —OH, halo, —CN, —CO₂R¹², —C(═O)NHR¹³, —NHR¹², C₁-C₆ alkyl or alkoxy; or two R¹⁰ taken together form oxo or ═N—OR¹¹;
R¹¹ and R¹³ are each independently H, —OH, or C₁-C₆ alkyl;
R¹², at each occurrence, is independently H, C₁-C₆ alkyl, aryl, cycloalkyl, or heterocyclyl;
R¹⁴, at each occurrence, is independently H or C₁-C₆ alkyl;
m, at each occurrence, is independently 0, 1 or 2; and
x, and y are each independently 0 or 1, wherein x and y are selected such that the sum of x+y is 0 or 1;

with the proviso that R^{3a} and R^{3b} are not cyclopropyl and methyl, respectively, when R¹ and W are each unsubstituted phenyl, x is 0 or 1, y is 0, and [symbol] is —C(R¹⁰)₂—C(R¹⁴)₂—; and

with the proviso that R^{3a} and R^{3b} taken together with the carbon to which they are attached do not form tetrahydrothiophene 1,1-dioxide or tetrahydrothiophene when at least one of R¹ and W is unsubstituted phenyl.

[symbol]

In one embodiment of Formula (Xa) or (Xb), [symbol] is —C(R¹⁰)₂—C(R¹⁴)₂—, —O—C(R¹⁴)₂—, O—C(O)—, —S(O)₂—C(R¹⁴)₂—, —S—C(R¹⁴)₂—, —NR⁹—C(O)—, —NR⁹—C(R¹⁴)₂—, —C(R¹⁰)₂—O—, —C(R¹⁰)₂—, or —C(R¹⁰)═C(R¹⁴)—. In another embodiment of Formula (Xa) or (Xb), [symbol] is —C(R¹⁰)₂—C(R¹⁴)₂—. In another embodiment of Formula (Xa) or (Xb), [symbol] is —O—C(R¹⁴)₂—. In another embodiment of Formula (Xa) or (Xb), [symbol] is —O—C(O)—. In another embodiment of Formula (Xa) or (Xb), [symbol] is —S(O)₂—C(R¹⁴)₂—. In another embodiment of Formula (Xa) or (Xb), [symbol] is —S—C(R¹⁴)₂—. In another embodiment of Formula (Xa) or (Xb), [symbol] is —NR⁹—C(O)—. In another embodiment of Formula (Xa) or (Xb), [symbol] is —NR⁹—C(R¹⁴)₂—. In another embodiment of Formula (Xa) or (Xb), [symbol] is —C(R¹⁰)₂—O—. In another embodiment of Formula (Xa) or (Xb), [symbol] is —C(R¹⁰)₂—. In another embodiment of Formula (Xa) or (Xb), [symbol] is —C(R¹⁰)═C(R¹⁴)—.

In one embodiment of Formula (Xa) or (Xb), W is arylene or heteroarylene. In another embodiment of Formula (Xa) or (Xb), W is an arylene. In another embodiment of Formula (Xa) or (Xb), W is heteroarylene. In another embodiment of Formula (Xa) or (Xb), W is

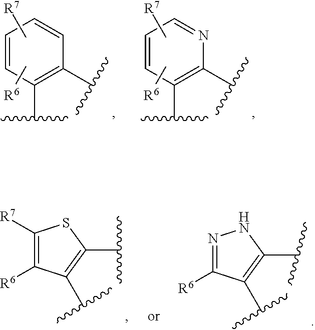

In another embodiment of Formula (Xa) or (Xb), W is

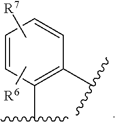

In another embodiment of Formula (Xa) or (Xb), W is

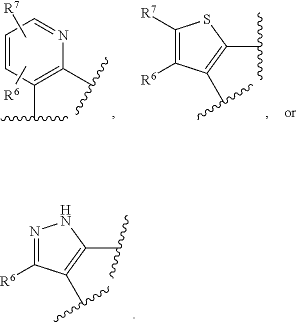

In one embodiment of Formula (Xa) or (Xb), R¹ is carbocyclyl or heterocyclyl. In another embodiment of Formula (Xa) or (Xb), R¹ is carbocyclyl. In another embodiment of Formula (Xa) or (Xb), R¹ is heterocyclyl. In another embodiment of Formula (Xa) or (Xb), R¹ is phenyl, naphthyl, cyclopropyl, or cyclobutyl. In another embodiment of Formula (Xa) or (Xb), R¹ is phenyl. In another embodiment of Formula (Xa) or (Xb), R¹ is phenyl, which is unsubstituted. In another embodiment of Formula (Xa) or (Xb), R¹ is phenyl, which is substituted. In another embodiment of Formula (Xa) or (Xb), R¹ is phenyl, which is substituted with F, Cl, Br, CN, —NR_{g}Rₕ, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, aryl, and/or haloalkyl; wherein alkyl, alone or part of a group, is optionally substituted with F, C(═O)NR_{g}Rₕ, or CN. In another embodiment of Formula (Xa) or (Xb), R¹ is phenyl, which is substituted with F, Cl, Br, CN, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, and/or haloalkyl; wherein alkyl, alone or part of a group, is optionally substituted with F. In another embodiment of Formula (Xa) or (Xb), R¹ is phenyl, which is substituted with F. In another embodiment of Formula (Xa) or (Xb), R¹ is tetrahydropyranyl, thiazolyl, oxazolyl, thiadiazolyl, pyrimidinyl, thiophenyl, furanyl, triazolyl, indolyl, imidazolyl, or pyridinyl. In another embodiment of Formula (Xa) or (Xb), R¹ is tetrahydropyranyl, thiazolyl, oxazolyl, thiadiazolyl, pyrimidinyl, thiophenyl, furanyl, triazolyl, indolyl, imidazolyl, or pyridinyl; which is unsubstituted. In another embodiment of Formula (Xa) or (Xb), R¹ is tetrahydropyranyl, thiazolyl, oxazolyl, thiadiazolyl, pyrimidinyl, thiophenyl, furanyl, triazolyl, indolyl, imidazolyl, or pyridinyl; which is substituted. In another embodiment of Formula (Xa) or (Xb), R¹ is tetrahydropyranyl, thiazolyl, oxazolyl, thiadiazolyl, pyrimidinyl, thiophenyl, furanyl, triazolyl, indolyl, imidazolyl, or pyridinyl; which is substituted with F, Cl, Br, CN, —NR_{g}Rₕ, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, aryl, and/or haloalkyl; wherein alkyl, alone or part of a group, is optionally substituted with F, —C(═O)NR_{g}Rₕ, or CN.

In one embodiment of Formula (Xa) or (Xb), R^{2a} and R^{2b} are each independently H, D, or C₁-C₆ alkyl. In another embodiment of Formula (Xa) or (Xb), R^{2a} and R^{2b} are each independently H. In another embodiment of Formula (Xa) or (Xb), R^{2a} is H and R^{2b} is C₁-C₆ alkyl. In another embodiment of Formula (Xa) or (Xb), R^{2a} is H and R^{2b} is C₁-alkyl.

In one embodiment of Formula (Xa) or (Xb), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; or R^{3a} and R^{3b} taken together with the carbon to which they are attached form arene, cycloalkane, or heterocycle. In another embodiment of Formula (Xa) or (Xb), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; wherein C₁-C₆ alkyl, is optionally substituted with F, —CN, —NR_{g}Rₕ, —OR_{g}, or —SO₂R_{g}; wherein R_{g} and Rₕ are the same or different and independently hydrogen or alkyl; wherein aryl, cycloalkyl and heterocyclyl are optionally substituted with alkyl, —SO₂NR_{g}C(═O)ORₕ, —SO₂NR_{g}Rₕ, —C(═O)R_{g}, —C(═O)OR_{g}, or —C(═O)NR_{g}Rₕ; wherein R_{g} and Rₕ are the same or different and independently hydrogen or alkyl; wherein alkyl, alone or part of a group, is optionally substituted with aryl or —C(═O)OH.

In another embodiment of Formula (Xa) or (Xb), R^{3a} is C₁-C₆ alkyl; and R^{3b} is C₁-C₆ alkyl; wherein the C₁-C₆ alkyl is optionally substituted with one or more F, —CN, alkoxy, or —SO₂R_{g}. In another embodiment of Formula (Xa) or (Xb), R^{3a} is C₁-C₆ alkyl; and R^{3b} is CF₃. In another embodiment of Formula (Xa) or (Xb), R^{3a} is CH₃; and R^{3b} is CF₃. In another embodiment of Formula (Xa) or (Xb), R^{3a} is hydrogen; and R^{3b} is C₁-C₆ alkyl; wherein the C₁-C₆ alkyl is optionally substituted with one or more F, —CN, alkoxy, or —SO₂R_{g}. In another embodiment of Formula (Xa) or (Xb), R^{3a} is hydrogen; and R^{3b} is cycloalkyl. In another embodiment of Formula (Xa) or (Xb), R^{3a} is hydrogen; and R^{3b} is cyclopropyl. In another embodiment of Formula (Xa) or (Xb), R^{3a} is C₁-C₆ alkyl; wherein the C₁-C₆ alkyl is CH₃; and R^{3b} is cycloalkyl. In another embodiment of Formula (Xa) or (Xb), R^{3a} is CH₃; and R^{3b} is cyclopropyl. In another embodiment of Formula (Xa) or (Xb), R^{3a} is CF₃; and R^{3b} is cyclopropyl. In another embodiment of Formula (Xa) or (Xb), R^{3a} is cycloalkyl; and R^{3b} is cycloalkyl. In another embodiment of Formula (Xa) or (Xb), R^{3a} is cyclopropyl; and R^{3b} is cyclopropyl. In another embodiment of Formula (Xa) or (Xb), R^{3a} is C₁-C₆ alkyl; and R^{3b} is heterocyclyl. In another embodiment of Formula (Xa) or (Xb), R^{3a} is hydrogen; and R^{3b} is aryl. In another embodiment of Formula (Xa) or (Xb), R^{3a} and R^{3b} taken together with the carbon to which they are attached form arene, cycloalkane, or heterocycle. In another embodiment of Formula (Xa) or (Xb), R^{3a} and R^{3b} taken together with the carbon to which they are attached form arene. In another embodiment of Formula (Xa) or (Xb), R^{3a} and R^{3b} taken together with the carbon to which they are attached form cycloalkane. In another embodiment of Formula (Xa) or (Xb), R^{3a} and R^{3b} taken together with the carbon to which they are attached form heterocycle. In another embodiment of Formula (Xa) or (Xb), R^{3a} and R^{3b} taken together with the carbon to which they are attached form cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, or benzene. In another embodiment of Formula (Xa) or (Xb), R^{3a} and R^{3b} taken together with the carbon to which they are attached form cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, or benzene wherein cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, and benzene are optionally substituted with alkyl, haloalkyl, F, CN, —NR_{g}Rₕ, —NR_{g}C(═O)Rₕ, —NR_{g}SO₂Rₕ, —OR_{g}, —SO₂R_{g}, —NR_{g}SO₂NR_{g}C(═O)Rₕ, —SO₂NR_{g}C(═O)ORₕ—SO₂NR_{g}Rₕ, —C(═O)R_{g}, —C(═O)OR_{g}, or —C(═O)NR_{g}Rₕ; wherein R_{g} and Rₕ are the same or different and independently hydrogen, alkyl, or haloalkyl; wherein each alkyl, alone or part of a group, is optionally substituted with CN, alkyl, —SO₂NH₂, C(═O)NHCH₃, —C(═O)OC(CH₃)₃, —C(═O)OCH₃, or —C(═O)OH.

In one embodiment of Formula (Xa) or (Xb), R^{4a} and R^{4b} are each independently H, D or C₁-C₆ alkyl. In another embodiment of Formula (Xa) or (Xb), R^{4a} is H and R^{4b} is C₁-C₆ alkyl. In another embodiment of Formula (Xa) or (Xb), R^{4a} and R^{4b} are each independently H.

In one embodiment of Formula (Xa) or (Xb), R⁶ and R⁷ are each independently H, halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═S)N(R¹²)₂, —NHC(═N—C≡N)NR¹², —NHC(═N—C≡N)SR¹², or —NHS(O)ₘR¹². In another embodiment of Formula (Xa) or (Xb), R⁷ is H and R⁶ is H, halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═S)N(R¹²)₂, —NHC(═N—C≡N)NR¹², —NHC(═N—C≡N)SR¹², or —NHS(O)ₘR¹². In another embodiment of Formula (Xa) or (Xb), R⁷ is H and R⁶ is —N(R¹²)₂, —C(═O)NHR¹², —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═O)N(R¹²)₂, —NHC(═S)N(R¹²)₂, —NHC(═N—C≡N)NR¹², —NHC(═N—C≡N)SR¹², or —NHS(O)ₘR¹². In another embodiment of Formula (Xa) or (Xb), R⁷ is H and R⁶ is heterocyclyl. In another embodiment of Formula (Xa) or (Xb), R⁷ is H and R⁶ is pyrazolyl. In another embodiment of Formula (Xa) or (Xb), R⁷ is H and R⁶ is —NHC(═O)NHR¹².

In one embodiment of Formula (Xa) or (Xb), R⁹ is independently H or C₁-C₆ alkyl. In another embodiment of Formula (Xa) or (Xb), R⁹ is independently H. In another embodiment of Formula (Xa) or (Xb), R⁹ is independently C₁-C₆ alkyl.

In one embodiment of Formula (Xa) or (Xb), R¹⁰, at each occurrence, is independently H, —OH, halo, —CN, —CO₂R¹², —C(═O)NHR¹³, —NHR¹², C₁-C₆ alkyl or alkoxy; or two R¹⁰ taken together form oxo or ═N—OR¹¹. In another embodiment of Formula (Xa) or (Xb), R¹⁰, at each occurrence, is independently H, —OH, halo, —CN, —CO₂R¹², —C(═O)NHR¹³, —NHR¹², C₁-C₆ alkyl or alkoxy; or two R¹⁰ taken together form oxo or ═N—OR¹¹. In another embodiment of Formula (Xa) or (Xb), R¹⁰, at each occurrence, is independently H. In another embodiment of Formula (Xa) or (Xb), R¹⁰, at each occurrence, is independently H or —OH. In another embodiment of Formula (Xa) or (Xb), R¹⁰, at each occurrence, is independently H or halo. In another embodiment of Formula (Xa) or (Xb), R¹⁰, at each occurrence, is independently H or F. In another embodiment of Formula (Xa) or (Xb), one R¹⁰ is independently H, and the remaining is F. In another embodiment of Formula (Xa) or (Xb), R¹⁰, at each occurrence, is independently H or —CN. In another embodiment of Formula (Xa) or (Xb), R¹⁰, at each occurrence, is independently H or —CO₂R¹². In another embodiment of Formula (Xa) or (Xb), R¹⁰, at each occurrence, is independently H or —C(═O)NHR¹³. In another embodiment of Formula (Xa) or (Xb), R¹⁰, at each occurrence, is independently H or —NHR¹². In another embodiment of Formula (Xa) or (Xb), R¹⁰, at each occurrence, is independently H or C₁-C₆ alkyl. In another embodiment of Formula (Xa) or (Xb), R¹⁰, at each occurrence, is independently H or alkoxy. In another embodiment of Formula (Xa) or (Xb), two R¹⁰ taken together form oxo. In another embodiment of Formula (Xa) or (Xb), two R¹⁰ taken together form ═N—OR¹¹.

In one embodiment of Formula (Xa) or (Xb), R¹¹ and R¹³ are each independently H, —OH, or C₁-C₆ alkyl. In another embodiment of Formula (Xa) or (Xb), R¹¹ and R¹³ are each independently H. In another embodiment of Formula (Xa) or (Xb), R¹¹ and R¹³ are each independently —OH. In another embodiment of Formula (Xa) or (Xb), R¹¹ and R¹³ are each independently C₁-C₆ alkyl.

In one embodiment of Formula (Xa) or (Xb), R¹², at each occurrence, is independently H, C₁-C₆ alkyl, aryl, cycloalkyl, or heterocyclyl. In another embodiment of Formula (Xa) or (Xb), R¹², at each occurrence, is independently H, C₁-C₆ alkyl, aryl, cycloalkyl, or heterocyclyl; wherein C₁-C₆ alkyl, aryl, cycloalkyl, and heterocyclyl are optionally substituted with F, alkyl, alkoxy, cycloalkyl, haloalkyl, heterocyclyl, heterocyclylalkyl, oxo, CN, —NR_{g}Rₕ, —NR_{g}C(═O)Rₕ, —NR_{g}C(═O)ORₕ, —OR_{g}, —SO₂R_{g}, —SO₂NR_{g}Rₕ, —C(═O)R_{g}, —C(═O)OR_{g}, or —C(═O)NR_{g}Rₕ; wherein R_{g} and Rₕ are the same or different and independently hydrogen, alkyl, alkoxy, aryl, cycloalkyl, and/or haloalkyl.

In one embodiment of Formula (Xa) or (Xb), R¹⁴, at each occurrence, is independently H or C₁-C₆ alkyl. In another embodiment of Formula (Xa) or (Xb), R¹⁴, at each occurrence, is independently H. In another embodiment of Formula (Xa) or (Xb), R¹⁴, at each occurrence, is independently C₁-C₆ alkyl.

In one embodiment of Formula (Xa) or (Xb), m, at each occurrence, is independently 0, 1 or 2. In another embodiment of Formula (Xa) or (Xb), m, at each occurrence, is independently 0. In another embodiment of Formula (Xa) or (Xb), m, at each occurrence, is independently 1. In another embodiment of Formula (Xa) or (Xb), m, at each occurrence, is independently 2.

In one embodiment of Formula (Xa) or (Xb), x, and y are each independently 0 or 1, wherein x and y are selected such that the sum of x+y is 0 or 1. In another embodiment of Formula (Xa) or (Xb), x is 0 and y is 0. In another embodiment of Formula (Xa) or (Xb), x is 0 and y is 1. In another embodiment of Formula (Xa) or (Xb), x is 1 and y is 0.

Still another embodiment pertains to compounds of Formula (Xa), selected from the group consisting of:
N-benzyl-N-(1-cyclopropylethyl)-2-(2′,4′-dioxo-2,3-dihydrospiro[indene-1,5′-ozazolidine]-3′-yl)acetamide;
N-benzyl-2-(5-bromo-2′,4′-dioxo-2,3-dihydrospiro[indene-1,5′-ozazolidine]-3′-yl)-N-(1-cyclopropylethyl)acetamide;
N-benzyl-2-(2′,4′-dioxo-2,3-dihydrospiro[indene-1,5′-ozazolidine]-3′-yl)-N-(pentan-3-yl)acetamide;
N-benzyl-N-(1-cyclobutylethyl)-2-(2′,4′-dioxo-2,3-dihydrospiro[indene-1,5′-ozazolidine]-3′-yl)acetamide;
N-(1-cyclopropylethyl)-2-(2′,4′-dioxo-2,3-dihydrospiro[indene-1,5′-ozazolidine]-3′-yl)-N-(4-fluorobenzyl)acetamide;
N-benzyl-N-(1-cyclopropylethyl)-2-(4-methoxy-2′,4′-dioxo-2,3-dihydrospiro[indene-1,5′-ozazolidine]-3′-yl)acetamide;
2-(5-bromo-2′,4′-dioxo-2,3-dihydrospiro[indene-1,5′-ozazolidine]-3′-yl)-N-(1-cyclopropylethyl)-N-(4-fluorobenzyl)acetamide;
N-benzyl-2-(5-cyano-2′,4′-dioxo-2,3-dihydrospiro[indene-1,5′-ozazolidine]-3′-yl)-N-(1-cyclopropylethyl)acetamide;
(S)-2-(5-acetamido-2′,4′-dioxo-2,3-dihydrospiro[indene-1,5′-ozazolidine]-3′-yl)-N-benzyl-N-(1-cyclopropylethyl)acetamide;
3′-(2-(benzyl(1-cyclopropylethyl)amino)-2-oxoethyl)-2′,4′-dioxo-2,3-dihydrospiro[indene-1,5′-ozazolidine]-5-carboxamide;
N-benzyl-N-(1-cyclopropylethyl)-2-(5-methoxy-2′,4′-dioxo-2,3-dihydrospiro[indene-1,5′-ozazolidine]-3′-yl)acetamide;
N-benzyl-N—((S)-1-cyclopropylethyl)-2-(5-(3-hydroxyprop-1-ynyl)-2′,4′-dioxo-2,3-dihydrospiro[indene-1,5′-ozazolidine]-3′-yl)acetamide;
N-benzyl-N—((S)-1-cyclopropylethyl)-2-(5-(methylsulfonamido)-2′,4′-dioxo-2,3-dihydrospiro[indene-1,5′-ozazolidine]-3′-yl)acetamide;
(S)-3′-(2-(benzyl(1-cyclopropylethyl)amino)-2-oxoethyl)-2′,4′-dioxo-2,3-dihydrospiro[indene-1,5′-ozazolidine]-5-carboxamide;
N-benzyl-N—((S)-1-cyclopropylethyl)-2-(5-(morpholinomethyl)-2′,4′-dioxo-2,3-dihydrospiro[indene-1,5′-ozazolidine]-3′-yl)acetamide;
N-benzyl-N—((S)-1-cyclopropylethyl)-2-(5-(3-methylureido)-2′,4′-dioxo-2,3-dihydrospiro[indene-1,5′-ozazolidine]-3′-yl)acetamide;
(S)—N-(3′-(2-(benzyl(1-cyclopropylethyl)amino)-2-oxoethyl)-2′,4′-dioxo-2,3-dihydrospiro[indene-1,5′-ozazolidine]-5-yl)-3-oxobutanamide;
N-benzyl-N—((S)-1-cyclopropylethyl)-2-(5-morpholino-2′,4′-dioxo-2,3-dihydrospiro[indene-1,5′-ozazolidine]-3′-yl)acetamide;
N-benzyl-2-(5-(2-cyanoacetamido)-2′,4′-dioxo-2,3-dihydrospiro[indene-1,5′-ozazolidine]-3′-yl)-N—((S)-1-cyclopropylethyl)acetamide;
N-benzyl-N—((S)-1-cyclopropylethyl)-2-(2′,4′-dioxo-5-(2-oxa-6-azaspiro[3.3]heptan-6-yl)-2,3-dihydrospiro[indene-1,5′-ozazolidine]-3′-yl)acetamide;
N-benzyl-N—((S)-1-cyclopropylethyl)-2-(2′,4′-dioxo-5-(piperidin-1-ylmethyl)-2,3-dihydrospiro[indene-1,5′-ozazolidine]-3′-yl)acetamide;
N-benzyl-N—((S)-1-cyclopropylethyl)-2-(2′,4′-dioxo-5-(piperazin-1-ylmethyl)-2,3-dihydrospiro[indene-1,5′-ozazolidine]-3′-yl)acetamide;
N-benzyl-N—((S)-1-cyclopropylethyl)-2-(5-((4-hydroxypiperidin-1-yl)methyl)-2′,4′-dioxo-2,3-dihydrospiro[indene-1,5′-ozazolidine]-3′-yl)acetamide;
(S)-2-amino-N-(3′-(2-(benzyl(1-cyclopropylethyl)amino)-2-oxoethyl)-2′,4′-dioxo-2,3-dihydrospiro[indene-1,5′-ozazolidine]-5-yl)-2-methylpropanamide;
(S)—N-benzyl-N-(1-cyclopropylethyl)-2-(2′,4′-dioxo-5-(piperazin-1-yl)-2,3-dihydrospiro[indene-1,5′-ozazolidine]-3′-yl)acetamide;
N-benzyl-N—((S)-1-cyclopropylethyl)-2-(5-hydroxy-2′,4′-dioxo-2,3-dihydrospiro[indene-1,5′-ozazolidine]-3′-yl)acetamide;
methyl 2-(3′-(2-(benzyl((S)-1-cyclopropylethyl)amino)-2-oxoethyl)-2′,4′-dioxo-2,3-dihydrospiro[indene-1,5′-ozazolidine]-5-yloxyacetate;
N-benzyl-N—((S)-1-cyclopropylethyl)-2-(2′,4′-dioxo-5-(prop-2-ynyloxy)-2,3-dihydrospiro[indene-1,5′-ozazolidine]-3′-yl)acetamide;
(S)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)-3-methylbutanamide;
(2S)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-ylpropanamide;
2-(3′-(2-(benzyl((S)-1-cyclopropylethyl)amino)-2-oxoethyl)-2′,4′-dioxo-2,3-dihydrospiro[indene-1,5′-ozazolidine]-5-ylamino)acetic acid;
N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-cyano-3-oxobutanamide;
2-(3′-(2-(benzyl((S)-1-cyclopropylethyl)amino)-2-oxoethyl)-2′,4′-dioxo-2,3-dihydrospiro[indene-1,5′-ozazolidine]-5-yloxy)acetic acid;
N-benzyl-N—((S)-1-cyclopropylethyl)-2-(5-(oxetan-3-ylamino)-2′,4′-dioxo-2,3-dihydrospiro[indene-1,5′-ozazolidine]-3′-yl)acetamide;
N-benzyl-N—((S)-1-cyclopropylethyl)-2-(2′,4′-dioxo-5-(tetrahydro-2H-pyran-4-ylamino)-2,3-dihydrospiro[indene-1,5′-ozazolidine]-3′-yl)acetamide;
N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-hydroxypropanamide;
1-(3′-(2-(benzyl((S)-1-cyclopropylethyl)amino)-2-oxoethyl)-2′,4′-dioxo-2,3-dihydrospiro[indene-1,5′-ozazolidine]-5-yl)azetidine-3-carboxamide;
2-amino-N-(3′-(2-(benzyl(dicyclopropylmethyl)amino)-2-oxoethyl)-2′,4′-dioxo-2,3-dihydrospiro[indene-1,5′-ozazolidine]-5-yl)-2-methylpropanamide;
N-benzyl-N-(dicyclopropylmethyl)-2-(5-(3-methylureido)-2′,4′-dioxo-2,3-dihydrospiro[indene-1,5′-ozazolidine]-3′-yl)acetamide;
N-benzyl-N—((S)-1-cyclopropylethyl)-2-(5-(2-(dimethylamino)ethoxy)-2′,4′-dioxo-2,3-dihydrospiro[indene-1,5′-ozazolidine]-3′-yl)acetamide;
2-(5-((1H-1,2,3-triazol-4-yl)methoxy)-2′,4′-dioxo-2,3-dihydrospiro[indene-1,5′-ozazolidine]-3′-yl)-N-benzyl-N—((S)-1-cyclopropylethyl)acetamide;
N-benzyl-N—((S)-1-cyclopropylethyl)-2-(2′,4′-dioxo-5-(6-oxo-1,6-dihydropyridin-3-yl)-2,3-dihydrospiro[indene-1,5′-ozazolidine]-3′-yl)acetamide;
N-benzyl-N—((S)-1-cyclopropylethyl)-2-(5-(2-hydroxyethoxy)-2′,4′-dioxo-2,3-dihydrospiro[indene-1,5′-ozazolidine]-3′-yl)acetamide;
3′-(2-(benzyl((S)-1-cyclopropylethyl)amino)-2-oxoethyl)-2′,4′-dioxo-2,3-dihydrospiro[indene-1,5′-ozazolidine]-5-ylboronic acid;
N-benzyl-N—((S)-1-cyclopropylethyl)-2-(5-(3-(hydroxymethyl)azetidin-3-yl)-2′,4′-dioxo-2,3-dihydrospiro[indene-1,5′-ozazolidine]-3′-yl)acetamide;
N—((S)-1-cyclopropylethyl)-2-(2′,4′-dioxo-5-(6-oxo-1,6-dihydropyridin-3-yl)-2,3-dihydrospiro[indene-1,5′-ozazolidine]-3′-yl)-N-(4-fluorobenzyl)acetamide;
1-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)cyclobutanecarboxamide;
2-(5-(2-cyanoacetamido)-2′,4′-dioxo-2,3-dihydrospiro[indene-1,5′-ozazolidine]-3′-yl)-N—((S)-1-cyclopropylethyl)-N-(4-fluorobenzyl)acetamide;
N—((S)-1-cyclopropylethyl)-N-(4-fluorobenzyl)-2-(5-(3-methylureido)-2′,4′-dioxo-2,3-dihydrospiro[indene-1,5′-ozazolidine]-3′-yl)acetamide;
3′-((4S)-3-benzyl-4-cyclopropyl-2-oxopentyl)-5-(1H-pyrazol-5-yl)-2,3-dihydrospiro[indene-3,5′-ozazolidine]-2′,4′-dione;
2-amino-N-(3′-(2-(benzyl((S)-1-cyclopropylethyl)amino)-2-oxoethyl)-4′-oxo-2,3-dihydrospiro[indene-1,5′-ozazolidine]-5-yl)-3-hydroxypropanamide;
(S)—N-(3′-(2-(benzyl(1-cyclopropylethyl)amino)-2-oxoethyl)-2′,4′-dioxo-2,3-dihydrospiro[indene-1,5′-ozazolidine]-5-yl)-2-hydroxy-2-methylpropanamide;
1-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)cyclopropanecarboxamide;
2-amino-N-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)-2-methylpropanamide;
(S)-2-(5-(azetidin-3-ylamino)-2′,4′-dioxo-2,3-dihydrospiro[indene-1,5′-ozazolidine]-3′-yl)-N-benzyl-N-(1-cyclopropylethyl)acetamide;
(2R)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-ylpropanamide;
(S)-2-(3′-(2-(benzyl(1-cyclopropylethyl)amino)-2-oxoethyl)-2′,4′-dioxo-2,3-dihydrospiro[indene-1,5′-ozazolidine]-5-ylcarboxamido)acetic acid;
2-((S)-5-acetamido-2′,4′-dioxo-2,3-dihydrospiro[indene-1,5′-ozazolidine]-3′-yl)-N-benzyl-N—((S)-1-cyclopropylethyl)acetamide;
2-((R)-5-acetamido-2′,4′-dioxo-2,3-dihydrospiro[indene-1,5′-ozazolidine]-3′-yl)-N-benzyl-N—((S)-1-cyclopropylethyl)acetamide;
N-benzyl-2-{5-[(methylcarbamoyl)amino]-3′,5′-dioxo-2,3-dihydrospiro[indene-1,2′-[1,4]ozazolidine]-4′-yl}-N-(pentan-3-yl)acetamide;
2-amino-N-(4′-{[benzyl(pentan-3-yl)carbamoyl]methyl}-3′,5′-dioxo-2,3-dihydrospiro[indene-1,2′-[1,4]ozazolidine]-5-yl)-2-methylpropanamide;
2-{5-[(azetidin-3-ylmethyl)amino]-3′,5′-dioxo-2,3-dihydrospiro[indene-1,2′-[1,4]ozazolidine]-4′-yl}-N-benzyl-N-[(1S)-1-cyclopropylethyl]acetamide;
N-benzyl-N-[(1S)-1-cyclopropylethyl]-2-{5-[(1,3-dihydroxypropan-2-yl)oxy]-3′,5′-dioxo-2,3-dihydrospiro[indene-1,2′-[1,4]ozazolidine]-4′-yl}acetamide;
1-amino-N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]cyclobutane-1-carboxamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]azetidine-3-carboxamide;
4-{[4′-({benzyl[(1S)-1-cyclopropylethyl]carbamoyl}methyl)-3′,5′-dioxo-2,3-dihydrospiro[indene-1,2′-[1,4]ozazolidine]-5-ylmethyl]amino}benzoic acid;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-2,3-dihydroxypropanamide;
N-benzyl-2-{5-[(methylcarbamoyl)amino]-3′,5′-dioxo-2,3-dihydrospiro[indene-1,2′-[1,4]ozazolidine]-4′-yl}-N-(oxolan-3-yl)acetamide;
N-benzyl-N-[(1S)-1-cyclopropylethyl]-2-{5-[(1,1-dioxo-1λ⁶-thian-4-yl)amino]-3′,5′-dioxo-2,3-dihydrospiro[indene-1,2′-[1,4]ozazolidine]-4′-yl}acetamide;
2-{5-[(2R)-2-amino-2-cyclopropylacetamido]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-(cyclopropylmethyl)-2-{5-[(methylcarbamoyl)amino]-3′,5′-dioxo-2,3-dihydrospiro[indene-1,2′-[1,4]ozazolidine]-4′-yl}-N-[(2-methylphenyl)methyl]acetamide;
N-ethyl-2-{5-[(methylcarbamoyl)amino]-3′,5′-dioxo-2,3-dihydrospiro[indene-1,2′-[1,4]ozazolidine]-4′-yl}-N-[(2-methylphenyl)methyl]acetamide;
N-[(1S)-1-cyclopropylethyl]-2-{3′,5′-dioxo-5-[(pyrrolidin-3-yl)amino]-2,3-dihydrospiro[indene-1,2′-[1,4]ozazolidine]-4′-yl}-N-[(4-fluorophenyl)methyl]acetamide;
2-(5-{[(1-aminocyclopropyl)methyl]amino}-3′,5′-dioxo-2,3-dihydrospiro[indene-1,2′-[1,4]ozazolidine]-4′-yl)-N-[(1S)-1-cyclopropylethyl]-N-[(4-fluorophenyl)methyl]acetamide;
N-benzyl-2-{5-[(methylcarbamoyl)amino]-3′,5′-dioxo-2,3-dihydrospiro[indene-1,2′-[1,4]ozazolidine]-4′-yl}-N-(oxan-4-yl)acetamide;
N-benzyl-N-[(1R)-1-cyclopropyl-2,2,2-trifluoroethyl]-2-{5-[(methylcarbamoyl)amino]-3′,5′-dioxo-2,3-dihydrospiro[indene-1,2′-[1,4]ozazolidine]-4′-yl}acetamide;
N-benzyl-2-{5-[(methylcarbamoyl)amino]-3′,5′-dioxo-2,3-dihydrospiro[indene-1,2′-[1,4]ozazolidine]-4′-yl}-N-(oxan-3-yl)acetamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]ethanediamide;
2-{5-[(azetidin-3-yl)amino]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-benzyl-N-[(1S)-1-cyclopropylethyl]-2-{3′,5′-dioxo-5-[(2-oxopyrrolidin-3-yl)amino]-2,3-dihydrospiro[indene-1,2′-[1,4]ozazolidine]-4′-yl}acetamide;
2-{[4′-({benzyl[(1S)-1-cyclopropylethyl]carbamoyl}methyl)-3′,5′-dioxo-2,3-dihydrospiro[indene-1,2′-[1,4]ozazolidine]-5-yl]carbamoyl}-2,2-dimethylacetic acid;
N-benzyl-N-[(1S)-1-cyclopropylethyl]-2-{5-[(2-hydroxyethyl)amino]-3′,5′-dioxo-2,3-dihydrospiro[indene-1,2′-[1,4]ozazolidine]-4′-yl}acetamide;
(2R)-2-amino-N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]propanamide;
{[4′-({benzyl[(1S)-1-cyclopropylethyl]carbamoyl}methyl)-3′,5′-dioxo-2,3-dihydrospiro[indene-1,2′-[1,4]ozazolidine]-5-yl]carbamoyl}formic acid;
(2S)-2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-hydroxypropanamide;
N-[(1S)-1-cyclopropylethyl]-2-{3′,5′-dioxo-5-[(piperidin-3-yl)amino]-2,3-dihydrospiro[indene-1,2′-[1,4]ozazolidine]-4′-yl}-N-[(4-fluorophenyl)methyl]acetamide;
N-benzyl-N-[(1S)-1-cyclopropylethyl]-2-{5-[(methylcarbamoyl)amino]-3′,5′-dioxo-2,3-dihydro(3,3-²H₂)spiro[indene-1,2′-[1,4]ozazolidine]-4′-yl}acetamide;
N-[(4-fluorophenyl)methyl]-2-{5-[(methylcarbamoyl)amino]-3′,5′-dioxo-2,3-dihydrospiro[indene-1,2′-[1,4]ozazolidine]-4′-yl}-N-[(2S)-1,1,1-trifluoropropan-2-yl]acetamide;
2-amino-N-[4′-({benzyl[(1R)-1-cyclopropyl-2,2,2-trifluoroethyl]carbamoyl}methyl)-3′,5′-dioxo-2,3-dihydrospiro[indene-1,2′-[1,4]ozazolidine]-5-yl]-2-methylpropanamide;
2-{5-[(methylcarbamoyl)amino]-3′,5′-dioxo-2,3-dihydrospiro[indene-1,2′-[1,4]ozazolidine]-4′-yl}-N-[(2-methylphenyl)methyl]-N-(2-methylpropyl)acetamide;
2-{5-[(4-aminooxolan-3-yl)amino]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
2-[(4-²H)benzyl]-N-[(1S)-1-cyclopropylethyl]-2-{5-[(methylcarbamoyl)amino]-3′,5′-dioxo-2,3-dihydrospiro[indene-1,2′-[1,4]ozazolidine]-4′-yl}acetamide;
N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-2-methyl-2-(methylamino)propanamide;
(2R)-2-amino-N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-benzyl-N-[(1S)-1-cyclopropylethyl]-2-[(1S)-5-[(methylcarbamoyl)amino]-3′,5′-dioxo-2,3-dihydrospiro[indene-1,2′-[1,4]ozazolidine]-4′-yl]acetamide;
N-benzyl-N-[(1S)-1-cyclopropylethyl]-2-[(1R)-5-[(methylcarbamoyl)amino]-3′,5′-dioxo-2,3-dihydrospiro[indene-1,2′-[1,4]ozazolidine]-4′-yl]acetamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-methylazetidine-3-carboxamide;
N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylazetidine-3-carboxamide;
2-{5-[(2R)-2-amino-2-cyclopropylacetamido]-3′,5′-dioxo-2,3-dihydrospiro[indene-1,2′-[1,4]ozazolidine-]4′-yl}-N-(4-fluorophenyl)methyl]-N-(2S)-1,1,1-trifluoropropan-2-yl]acetamide;
N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylazetidine-3-carboxamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-2-(dimethylamino)-2-methylpropanamide;
(2R)-2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-hydroxypropanamide;
2-{5-bromo-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
2-{5-[(3-aminocyclohexyl)amino]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-[1-(azetidin-3-yl)ethyl]-N-benzyl-2-{5-[(methylcarbamoyl)amino]-3′,5′-dioxo-2,3-dihydrospiro[indene-1,2′-[1,4]ozazolidine-4′-yl}acetamide;
2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-2-methylpropanamide;
N-[(4-fluorophenyl)methyl]-N-[(2S)-1-methoxypropan-2-yl]-2-{5-[(methylcarbamoyl)amino]-3′,5′-dioxo-2,3-dihydrospiro[indene-1,2′-[1,4]ozazolidine]-4′-yl}acetamide;
3-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]oxetane-3-carboxamide;
2-amino-N-[(1S)-4′-({[(4-bromophenyl)methyl [(1S)-1-cyclopropylethyl]carbamoyl}methyl)-3′,5′-dioxo-2,3-dihydrospiro[indene-1,2′-[1,4]ozazolidine]-5-yl]-2-methylpropanamide;
2-amino-N-[(1R)-4′-({[(4-bromophenyl)methyl][(1S)-1-cyclopropylethyl]carbamoyl}methyl)-3′,5′-dioxo-2,3-dihydrospiro[indene-1,2′-[1,4]ozazolidine)-5-yl]-2-methylpropanamide;
2-{5-[(2R)-2-amino-2-(oxetan-3-yl)acetamido]-3′,5′-dioxo-2,3-dihydrospiro[indene-1,2′-[1,4]ozazolidine]-4′-yl}-N-benzyl-N-[(1S)-1-cyclopropylethyl]acetamide;
1-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3-difluorocyclobutane-1-carboxamide;
2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1S)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
(2R)-2-amino-N-[{(1R)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(4-fluorophenyl)methyl]-2-[(1S)-5-[(methylcarbamoyl)amino]-3′,5′-dioxo-2,3-dihydrospiro[indene-[1,2′-[1,4]ozazolidine]-4′-yl]-N-[(2S)-1,1,1-trifluoropropan-2-yl]acetamide;
N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]-N-[(2S)-1,1,1-trifluoropropan-2-yl]acetamide;
N-[(1S)-1-cyclopropylethyl]-N-(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]acetamide;
N-[(1S)-1-cyclopropylethyl]-N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]acetamide;
(2S)-2-amino-N-[(1S)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1S)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2S)-2-amino-N-[(1R)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1R)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
N-benzyl-N-[(1R)-1-cyclopropyl-2,2,2-trifluoroethyl]-2-[(1S)-5-[(methylcarbamoyl)amino]-3′,5′-dioxo-2,3-dihydrospiro[indene-1,2′-[1,4]ozazolidine]-4′-yl]acetamide;
N-benzyl-N-[(1R)-1-cyclopropyl-2,2,2-trifluoroethyl]-2-[(1R)-5-[(methylcarbamoyl)amino]-3′,5′-dioxo-2,3-dihydrospiro[indene-1,2′-[1,4]ozazolidine]-4′-yl]acetamide;
(2R)-2-amino-N-[(1S)-4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1R)-4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1S)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine-5-yl]-3-methylbutanamide;
(2R)-2-amino-N-[(1R)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(1R)-1-cyclopropyl-2,2,2-trifluoroethyl]-N-(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]acetamide;
N-[(1R)-1-cyclopropyl-2,2,2-trifluoroethyl]-N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]acetamide;
N-[(1S)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
N-[(1R)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
(2R)-2-amino-N-[4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(1S)-4′-({(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
N-[(1R)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
(2S)-2-amino-N-[4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
(2S)-2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]azetidine-3-carboxamide;
N-benzyl-N-[1-(1-methylazetidin-3-yl)ethyl]-2-{5-[(methylcarbamoyl)amino]-3′,5′-dioxo-2,3-dihydrospiro[indene-1,2′-[1,4]ozazolidine]-4′-yl}acetamide;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1R)-3′-(2-{[(1R)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-benzyl-2-[(1R)-5-bromo-2′,4′-dioxo-2,3-dihydro-3′H-spiro[indene-1,5′-[1,3]ozazolidin]-3′-yl]-N-[(1S)-1-cyclopropylethyl]acetamide;
2-[(1S)-5-amino-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-[(1R)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-[(1S)-3′-(2-{[(1R)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-[(1R)-1-cyclopropylethyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1R)-1-cyclopropylethyl]-N-(4-fluorobenzyl)-2-{(1S)-5-[(methylcarbamoyl)amino]-2′,4′-dioxo-2,3-dihydro-3′H-spiro[indene-1,5′-[1,3]ozazolidin]-3′-yl}acetamide;
N-[(1R)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-[(1S)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]azetidine-1-carboxylate;
2-[(1R)-5-(6-aminopyridin-3-yl)-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-{(1R)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-[(1S)-1-cyclopropylethyl]-N-(4-fluorobenzyl)acetamide;
N-[(1S)-1-cyclopropylethyl]-2-[(1R)-2′,4′-dioxo-5-(pyridin-3-yl)-2,3-dihydro-3′H-spiro[indene-1,5′-[1,3]ozazolidin]-3′-yl]-N-(4-fluorobenzyl)acetamide;
N-benzyl-2-(7-bromo-2′,4′-dioxo-2,3-dihydro-3′H-spiro[chromene-4,5′-[1,3]ozazolidin]-3′-yl)-N-[(1S)-1-cyclopropylethyl]acetamide;
2-[(1R)-5-(azetidin-3-yl)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
2-[(1R)-5-(1-acetylazetidin-3-yl)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-benzyl-N-[(1S)-1-cyclopropylethyl]-2-[(1R)-5-(1-methylazetidin-3-yl)-2′,4′-dioxo-2,3-dihydro-3′H-spiro[indene-1,5′-[1,3]ozazolidin]-3′-yl]acetamide;
3-[(1R)-3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]-N-methylazetidine-1-carboxamide;
N-benzyl-2-{(1R)-5-[1-(cyclopropylcarbonyl)azetidin-3-yl]-2′,4′-dioxo-2,3-dihydro-3′H-spiro[indene-1,5′-[1,3]ozazolidin]-3′-yl}-N-[(1S)-1-cyclopropylethyl]acetamide;
N-benzyl-N-[(1S)-1-cyclopropylethyl]-2-{(1R)-5-[1-(2-hydroxy-2-methylpropanoyl)azetidin-3-yl]-2′,4′-dioxo-2,3-dihydro-3′H-spiro[indene-1,5′-[1,3]ozazolidin]-3′-yl}acetamide;
N-benzyl-N-[(1S)-1-cyclopropylethyl]-2-{(1R)-5-[1-(methylsulfonyl)azetidin-3-yl]-2′,4′-dioxo-2,3-dihydro-3′H-spiro[indene-1,5′-[1,3]ozazolidin]-3′-yl}acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]cyclobutanecarboxamide;
3-ethoxy-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-5-oxo-D-prolinamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-5-oxo-L-prolinamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]cyclopropanecarboxamide;
N-(4-fluorobenzyl)-2-{(1R)-5-[(methoxyacetyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(1R)-5-[(ethoxyacetyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-3-methylbutanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylpropanamide;
2-[(1R)-5-(acetylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(7-amino-2′,4′-dioxo-2,3-dihydro-3′H-spiro[chromene-4,5′-[1,3]ozazolidin]-3′-yl)-N-benzyl-N-[(1S)-1-cyclopropylethyl]acetamide;
N-(4-fluorobenzyl)-2-[(1R)-5-{[(2-methoxyethyl)(methyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[(dimethylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-{[methyl(2-methylpropyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(cyclopropylmethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(cyclobutylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-({[(2R)-1-hydroxy-3-methylbutan-2-yl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-({[(2S)-tetrahydrofuran-2-ylmethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-({[(2R)-tetrahydrofuran-2-ylmethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-({[2-(propan-2-yloxy)ethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(cyclopropylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-{[ethyl(methyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(diethylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyrrolidine-1-carboxamide;
2-[(1R)-5-({[2-(dimethylamino)ethyl](methyl)carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N²-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-L-leucinamide;
N-(4-fluorobenzyl)-2-[(1R)-5-{[(2-hydroxyethyl)(propyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(2-methoxyethyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(3-hydroxypropyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-({[(2S)-1-hydroxy-3-methylbutan-2-yl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[methyl(propan-2-yl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(2-cyanoethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-{[methyl(propyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-benzyl-N-[(1S)-1-cyclopropylethyl]-2-{7′-[(methylcarbamoyl)amino]-2′,4′-dioxo-2,3-dihydro-3′H-spiro[chromene-4,5′-[1,3]ozazolidin]-3′-yl}acetamide;
2-[7-(acetylamino)-2′,4′-dioxo-2,3-dihydro-3′H-spiro[chromene-4,5′-[1,3]ozazolidin]-3′-yl]-N-benzyl-N-[(1S)-1-cyclopropylethyl]acetamide;
N-[(1S)-1-cyclopropylethyl]-2-[(1R)-2′,4′-dioxo-5-(pyridin-4-yl)-2,3-dihydro-3′H-spiro[indene-1,5′-[1,3]ozazolidin]-3′-yl]-N-(4-fluorobenzyl)acetamide;
N-[(1S)-1-cyclopropylethyl]-N-(4-fluorobenzyl)-2-[(1R)-5-(6-hydroxypyridazin-3-yl)-2′,4′-dioxo-2,3-dihydro-3′H-spiro[indene-1,5′-[1,3]ozazolidin]-3′-yl]acetamide;
N-[(1S)-1-cyclopropylethyl]-2-[(1R)-2′,4′-dioxo-5-(1H-pyrazol-4-yl)-2,3-dihydro-3′H-spiro[indene-1,5′-[1,3]ozazolidin]-3′-yl]-N-(4-fluorobenzyl)acetamide;
N-benzyl-N-[(1S)-1-cyclopropylethyl]-2-[2′,4′-dioxo-7-(6-oxo-1,6-dihydropyridin-3-yl)-2,3-dihydro-3′H-spiro[chromene-4,5′-[1,3]ozazolidin]-3′-yl]acetamide;
2-{(1R)-2′,4′-dioxo-5-[(tetrahydrofuran-2-ylmethyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[(cyclopentylmethyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[(2,2-dimethylbutyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-2′,4′-dioxo-5-[(tetrahydrofuran-3-ylmethyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-{(1R)-5-[(2-methyl)propyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
tert-butyl [(2R)-1-{[3′-(2-{benzyl[(1S)-1-cyclopropylethyl]amino}-2-oxoethyl)-2′,4′-dioxo-2,3-dihydrospiro[chromene-4,5′-[1,3]ozazolidin]-7-yl]amino}-3-methyl-1-oxobutan-2-yl]carbamate;
N-[3′-(2-{benzyl[(1S)-1-cyclopropylethyl]amino}-2-oxoethyl)-2′,4′-dioxo-2,3-dihydrospiro[chromene-4,5′-[1,3]ozazolidin]-7-yl]-D-valinamide;
2-[(1R)-6-amino-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
2-[(1R)-6-(acetylamino)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-benzyl-N-[(1S)-1-cyclopropylethyl]-2-{(1R)-6-[(methylcarbamoyl)amino]-2′,4′-dioxo-2,3-dihydro-3′H-spiro[indene-1,5′-[1,3]ozazolidin]-3′-yl}acetamide;
2-[(1S)-6-amino-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
2-[(1S)-6-(acetylamino)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-benzyl-N-[(1S)-1-cyclopropylethyl]-2-{(1S)-6-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-2-[(1R)-2′,4′-dioxo-5-(pyridin-2-yl)-2,3-dihydro-3′H-spiro[indene-1,5′-[1,3]ozazolidin]-3′-yl]-N-(4-fluorobenzyl)acetamide;
N-[(1S)-1-cyclopropylethyl]-N-(3-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropyl-2,2,2-trifluoroethyl]-N-(4-fluorobenzyl)-2-{7′-[(methylcarbamoyl)amino]-2,4-dioxo-2′,3′-dihydro-3′H-spiro[1,3-ozazolidine-5,4′-thiochromen]-3-yl}acetamide;
2-[(1R)-5-(carbamoylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1S)-1-cyclopropyl-2,2,2-trifluoroethyl]-N-(4-fluorobenzyl)-2-{7′-[(methylcarbamoyl)amino]-1′,1′-dioxido-2,4-dioxo-2′,3′-dihydro-3′H-spiro[1,3-ozazolidine-5,4′-thiochromen]-3-yl}acetamide;
N-(4-fluorobenzyl)-2-((R)-5-(3-(N-methylsulfamoyl)ureido)-2′,4′-dioxo-2,3-dihydrospiro[indene-1,5′-ozazolidine]-3-yl)-N—((S)-1,1,1-trifluoropropan-2-yl)acetamide
methyl {[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}sulfamate;
2-((R)-5-(3-(N-(cyclopropylmethyl)sulfamoyl)ureido)-2′,4′-dioxo-2,3-dihydrospiro[indene-1,5′-ozazolidine]-3′-yl)-N-(4-fluorobenzyl)-N—((S)-1,1,1-trifluoropropan-2-yl)acetamide
(1R)-3′-(2-{benzyl[(1S)-1-cyclopropylethyl]amino}-2-oxoethyl)-N-(2-methylpropyl)-2′,4′-dioxo-2,3-dihydrospiro[indene-1,5′-[1,3]ozazolidine]-5-carboxamide;
N-[(1S)-1-cyclopropylethyl]-N-(2-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,3-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-chloro-4-fluorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3-chloro-4-fluorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-[4-(trifluoromethyl)benzyl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1-methyl-1H-pyrazole-4-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1-methyl-1H-pyrazole-3-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-4-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-2-carboxamide;
2-(5-bromo-4-fluoro-2′,4′-dioxo-2,3-dihydro-3′H-spiro[indene-1,5′-[1,3]ozazolidin]-3′-yl)-M-[(1S)-1-cyclopropyl-2,2,2-trifluoroethyl]-N-(4-fluorobenzyl)acetamide;
2-(5-bromo-6-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
(1R)-3′-(2-{benzyl[(1S)-1-cyclopropylethyl]amino}-2-oxoethyl)-N-[(2R)-1-hydroxy-3-methylbutan-2-yl]-2′,4′-dioxo-2,3-dihydrospiro[indene-1,5′-[1,3]ozazolidine]-5-carboxamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-[(5-methyl-1,3,4-thiadiazol-2-yl)methyl]acetamide;
2-(5-amino-4-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{4-fluoro-5-[(methylcarbamoyl)amino]-2′,4′-dioxo-2,3-dihydro-3′H-spiro[indene-1,5′-[1,3]ozazolidin]-3′-yl}acetamide;
N-(4-cyanobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3-methylbenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,4-dichlorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-methoxybenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3-methoxybenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-methylbenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-[(1R)-5-{[(1-methyl-1H-pyrazol-4-yl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{7′-[1-(2-amino-2-oxoethyl)-1H-pyrazol-4-yl]-2′,4′-dioxo-2,3-dihydro-3′H-spiro[chromene-4,5′-[1,3]ozazolidin]-3′-yl}-N-benzyl-N-[(1S)-1-cyclopropylethyl]acetamide;
2-{(1R)-2′,4′-dioxo-5-[(phenylcarbamoyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-2′,4′-dioxo-5-[(pyridin-3-ylcarbamoyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-(5-amino-6-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{6-fluoro-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetamide;
N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(dimethylamino)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(4-methylpyrimidin-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[5-(difluoromethyl)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(6-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1R)-5-[1-(2-methyl)propyl)-1H-pyrazol-4-yl]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(1R)-5-[4-(difluoromethyl)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(3-fluoropyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(1,3-thiazol-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-N-methylbenzamide;
2-{(1R)-5-[2-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(5-cyanopyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(1-methyl-1H-1,2,4-triazol-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(5-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-(6-cyanopyridin-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(5-fluoro-3-methylpyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(5-methyl-1,3,4-thiadiazol-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(4-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(6-methoxypyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[2-(dimethylamino)pyrimidin-5-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(acetylamino)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(1-methyl-1H-1,2,4-triazol-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-(1R)-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-cyclopropyl-5-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]furan-2-carboxamide;
N-(4-fluorobenzyl)-2-[(3R)-5-(5-fluoropyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
4-fluoro-3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
5-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-N-methylpyridine-2-carboxamide;
6-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-3-carboxamide;
N-(4-fluorobenzyl)-2-[(1R)-5-(2-methoxypyrimidin-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
6-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-2-carboxamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]thiophene-3-carboxamide;
2-{(1R)-5-[4-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(1,3-dimethyl-2,4-dioxo-1,2,3,4-tetrahydropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4-cyanopyridin-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-4-carboxamide;
2-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
2-{(1R)-5-[3-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(6-cyano-5-methoxypyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(1-methyl-1H-pyrazol-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(1,3,5-trimethyl-1H-pyrazol-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(3,4-difluorophenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2,5-difluoro-4-methoxybenzyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyrimidin-5-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-2′,4′-dioxo-5-[5-(trifluoromethyl)pyridin-3-yl]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-2′,4′-dioxo-5-(1,3-thiazol-2-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2-cyanopyridin-4-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyrazin-2-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[4-(carbamoylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(2-cyanopyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(6-cyano-5-fluoropyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
2-[(1R)-5-{[(4-aminophenyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-{6-[(methylcarbamoyl)amino]-2′,4′-dioxo-3,4-dihydro-2H,3′H-spiro[naphthalene-1,5′-[1,3]ozazolidin]-3′-yl}-N-[(2S)-1,1,1-trifluoropropan-2-yl]acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(pyrimidin-5-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(pyrimidin-2-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-thiazol-5-ylmethyl)acetamide;
N-[(1R)-3′-(2-{(4-fluorobenzyl)[{28)-1,1,1-trifluoropropan-2-yl]amino}-2-oxoethyl}-2′,4′-dioxo-2,3-dihydrospiro[indene-1,5′-[1,3]ozazolidin]-5-yl]prolinamide;
N²-(4-fluorobenzyl)-N²-({(1R)-5-[(methylcarbamoyl)amino]-2′,4′-dioxo-2,3-dihydro-3′H-spiro[indene-1,5′-[1,3]ozazolidin]-3′-yl}acetyl)-L-alaninamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1H-pyrazole-5-carboxamide;
N-(4-fluorobenzyl)-2-[(1R)-5-{[(methylsulfamoyl)acetyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(methylsulfonyl)acetyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(dimethylsulfamoyl)acetyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-2′,4′-dioxo-5-[(sulfamoylacetyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidine-1-carboxamide
N-benzyl-N-ethyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
methyl N′-cyano-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamimidothioate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(tetrahydro-2H-pyran-4-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-oxazol-4-ylmethyl)acetamide;
2-[(1R)-5-(5-cyanothiophen-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4-cyano-3-fluorophenyl)-2′,4′-dioxo-2,3-dihydro-3′H-spiro[indene-1,5′-[1,3]ozazolidin]-3′-yl]-N-(4-fluorobenzyl)-N-(2S)-1,1,1-trifluoropropan-2-yl)acetamide;
2-[(1R)-5-(5-cyanothiophen-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(N″-cyano-N′-methylcarbamimidamido)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4,6-difluoropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[2-(acetylamino)-5-methylpyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(5-fluoropyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(furan-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(2-oxo-2,3-dihydro-1H-benzimidazol-5-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(4-methylpyrimidin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-2′,4′-dioxo-5-[1-(tetrahydro-2H-pyran-2-yl)-H-pyrazol-3-yl]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(5-fluoro-6-methylpyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[6-(difluoromethyl)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(difluoromethyl)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(furan-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[1-(ethoxymethyl)-1H-imidazol-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(5-cyano-1,2-dimethyl-6-oxo-1,6-dihydropyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]thiophene-2-carboxamide;
2-[(1R)-5-(2,6-dioxo-1,2,5,6-tetrahydropyrimidin-5-yl)-2′,4′-dioxo-2,3-dihydro-3′H-spiro[idene-1,5′-[1,3]ozazolidin]-3′-yl]-N-(4-fluorobenzyl)-N-[(2S)-1,1,1-trifluoropropan-2-yl]acetamide;
2-[(1R)-5-(2,4-dimethyl-1,3-thiazol-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[1-(cyanomethyl)-3,5-dimethyl-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[3-(carbamoylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-[3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)-2,2-dimethylpropyl]propanamide;
(1R)-3′-(2-{benzyl[(1S)-1-cyclopropylethyl]amino}-2-oxoethyl)-N-methyl-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(propan-2-yl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(2-methoxyethyl)-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(tetrahydrofuran-2-ylmethyl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(tetrahydrofuran-3-ylmethyl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(cyanomethyl)-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(3-hydroxypropyl)-2′,4′-dioxo-2,3-dihydrospiro[indene-1,5′-[1,3]ozazolidine]-5-carboxamide;
3′-{2-[benzyl(1-cyclopropylethyl)amino]-2-oxoethyl}-N-(1-hydroxy-3-methylbutan-2-yl)-2′,4′-dioxo-2,3-dihydrospiro[indene-1,5′-[1,3]ozazolidine]-5-carboxamide;
N-(1-amino-4-methyl-1-oxopentan-2-yl)-3′-{2-[benzyl(1-cyclopropylethyl)amino]-2-oxoethyl}-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-[2-(diethylamino)ethyl]-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-[3-(1H-imidazol-1-yl)propyl]-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-[3-(2-oxopyrrolidin-1-yl)propyl]-2,3-dihydrospiro[indene-1,5′-[1,3]ozazolidine]-5-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]tetrahydropyrimidine-1(2H)-carboxamide;
3-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]tetrahydropyrimidine-1(2H)-carboxamide;
N-(4-fluorobenzyl)-2-{(1R)-5-[1-(2-hydroxyethyl)-1H-pyrazol-4-yl]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-phenylacetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N,N-dibenzyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
(2R)-3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-hydroxy-2-methylpropanamide;
(2S)-3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-hydroxy-2-methylpropanamide;
2-[(1R)-2′,4′-dioxo-5-({[3-(pyrrolidin-1-ylmethyl)phenyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-({[3-(morpholin-4-ylmethyl)phenyl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N³-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-N-methyl-beta-alaninamide;
N-[3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)phenyl]propanamide;
2-(6-bromo-2′,4,4′-trioxo-3,4-dihydro-2H,3′H-spiro[naphthalene-1,5′-[1,3]ozazolidin]-3′-yl)-N-[(1S)-1-cyclopropyl-2,2,2-trifluoroethyl]-N-(4-fluorobenzyl)acetamide;
N-(2-chlorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)acetamide;
N-[(1S)-1-cyclopropyl-2,2,2-trifluoroethyl]-N-(4-fluorobenzyl)-2-{6-[(methylcarbamoyl)amino]-2′,4,4′-trioxo-3,4-dihydro-2H,3′H-spiro[naphthalene-1,5′-[1,3]ozazolidin]-3′-yl}acetamide;
N-(4-fluorobenzyl)-2-{(1S)-6-[(methylcarbamoyl)amino]-2′,4′-dioxo-3,4-dihydro-2H,3′H-spiro[naphthalene-1,5′-[1,3]ozazolidin]-3′-yl}-N-[(2S)-1,1,1-trifluoropropan-2-yl]acetamide;
N-(4-fluorobenzyl)-2-{(1R)-6-[(methylcarbamoyl)amino]-2′,4′-dioxo-3,4-dihydro-2H,3′H-spiro[naphthalene-1,5′-[1,3]ozazolidin]-3′-yl}-N-[(2S)-1,1,1-trifluoropropan-2-yl]acetamide;
2-[(1R)-5-(4,5-dihydro-1H-imidazol-2-ylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(6-amino-2′,4′-dioxo-3,4-dihydro-2H,3′H-spiro[naphthalene-1,5′-[1,3]ozazolidin]-3′-yl)-N-[(1S)-1-cyclopropyl-2,2,2-trifluoroethyl]-N-(4-fluorobenzyl)acetamide;
2-[(1R)-5-({[3-(acetylamino)propyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-({[2-(acetylamino)ethyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-yl]acetamide;
tert-butyl [3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)-2,2-dimethylpropyl]carbamate;
N-[2-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)ethyl]propanamide;
2-[(1R)-5-{[(3-amino-2,2-dimethylpropyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-({[3-(acetylamino)phenyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
methyl 2-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]amino}-4,5-dihydro-1H-imidazole-1-carboxylate;
2-{5-bromo-6-[(methylcarbamoyl)amino]-2′,4′-dioxo-3,4-dihydro-2H,3′H-spiro[naphthalene-1,5′-[1,3]ozazolidin]-3′-yl}-N-[(1S)-1-cyclopropyl-2,2,2-trifluoroethyl]-N-(4-fluorobenzyl)acetamide;
N-cyclohexyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-cyclopentyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropyl-2,2,2-trifluoroethyl]-N-(4-fluorobenzyl)-2-{4-hydroxy-6-[(methylcarbamoyl)amino]-2′,4′-dioxo-3,4-dihydro-2H,3′H-spiro[naphthalene-1,5′-[1,3]ozazolidin]-3′-yl}acetamide;
N-benzyl-N-[1-(furan-2-yl)ethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropyl-2,2,2-trifluoroethyl]-N-(4-fluorobenzyl)-2-{4-fluoro-6-[(methylcarbamoyl)amino]-2′,4′-dioxo-3,4-dihydro-2H,3′H-spiro[naphthalene-1,5′-[1,3]ozazolidin]-3′-yl}acetamide;
N-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-beta-alanine;
N-cyclopropyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl N-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-beta-alaninate;
N-(3,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(4-fluorobenzyl)-N-(3-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(1S)-5-amino-2′,4′-dioxo-2,3-dihydro-3′H-spiro[indene-1,5′-[1,3]ozazolidin]-3′-yl]-N-[(1R)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
ethyl ({(3R)-3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(3R)-piperidin-3-yl]acetamide;
ethyl ({(3R)-3-[{[(1R)-5-bromo-2′,4′-dioxo-2,3-dihydro-3′H-spiro[indene-1,3′-[1,3]ozazolidin]-3′-yl]acetyl}(4-fluorobenzyl)amino]piperidin-1-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(methylsulfonyl)piperidin-4-yl]acetamide;
N-(4-fluorobenzyl)-2-{6-fluoro-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-(5-amino-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1-phenylethyl)acetamide;
2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)-N-[2-(trifluoromethyl)benzyl]acetamide;
2-(5-bromo-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
N-benzyl-N-(cyanomethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-(2-cyanoethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(methylsulfonyl)propan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(1-methyl-1H-pyrazol-4-yl)methyl]acetamide;
N-[(1S)-1-cyclopropylethyl]-N-(4-fluorobenzyl)-2-{4-methoxy-5-[(methylcarbamoyl)amino]-2′,4′-dioxo-2,3-dihydro-3′H-spiro[indene-1,5′-[1,3]ozazolidin]-3′-yl}acetamide;
2-[5-(acetylamino)-4-methoxy-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-[(1R)-5-({[3-(acetylamino)-2,2-dimethylpropyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclopentyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(pyridin-3-yl)acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1-methyl-1H-pyrazol-4-yl)acetamide;
N-benzyl-N-(2,2-difluorocyclopentyl)-2-{5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-(2-methylcyclopropyl)acetamide;
N-(2,2-dimethylcyclopentyl)-N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-(2-methylcyclopentyl)acetamide;
2-(5-{[(2-cyanoethyl)carbamoyl]amino}-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
methyl [(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamate;
4-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)butanoic acid;
N²-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylalaninamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylalaninamide;
N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
N-cyclopentyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-oxazol-4-ylmethyl)acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-[1-(pyridin-2-yl)ethyl]acetamide;
N-(4-chlorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3,5-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-(5-{[(cyanomethyl)carbamoyl]amino}-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
2-[(1R)-5-{[(cyanomethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclohexyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-pyrrolidin-3-yl]acetamide;
N-(4-fluorobenzyl)-N-(trans-3-hydroxycyclobutyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-1-(methylsulfonyl)pyrrolidin-3-yl]acetamide;
ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidin-1-yl}sulfonyl)carbamate;
N-(1-cyanopropan-2-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
5-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)pentanoic acid;
N-[(1S)-1-cyclopropyl-2,2,2-trifluoroethyl]-N-(4-fluorobenzyl)-2-[4-fluoro-5-(1-methyl-1H-pyrazol-4-yl)-2′,4′-dioxo-2,3-dihydro-3′H-spiro[indene-1,5′-[1,3]ozazolidin]-3′-yl]acetamide;
N-(4-fluorobenzyl)-2-[6-fluoro-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-[(3R)-1-acetylpyrrolidin-3-yl]-N-(4-fluorobenzyl)-2-{(1R)-5-[(methylcarbonyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-1-(2-sulfamoylethyl)pyrrolidin-3-yl]acetamide;
N-(4-fluorobenzyl)-N-(trans-4-hydroxycyclohexyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
methyl (1R,3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-3-(methylsulfonyl)tetrahydropyrimidine-1(2H)-carboxamide;
N²-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]glycinamide;
2-{(1R)-5-[(methylcarbamoyl)amino]-2′,4′-dioxo-2,3-dihydro-3′H-spiro[indene-1,5′-[1,3]ozazolidin]-3′-yl}-N-[1-(2-methylphenyl)ethyl]-N-(2-methylpropyl)acetamide;
N-(2-chlorobenzyl)-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-fluorobenzyl)-2-{(1R)-5-[(methylcarbamoyl)amino]-2′,4′-dioxo-2,3-dihydro-3′H-spiro[indene-1,5′]-[1,3]ozazolidin]-3′-yl}-N-(2-methylpropyl)acetamide;
N-(4-fluorobenzyl)-N-(4-methoxyphenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(4-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(2-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(trifluoromethyl)cyclopropyl]acetamide;
2-[(1R)-5-(3,4-dihydro-2H-pyrrol-5-ylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(3,4-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(2,5-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3-chlorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{5-[1-(2-amino-2-oxoethyl)-1H-pyrazol-4-yl]-6-fluoro-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(3R)-pyrrolidin-3-yl]acetamide;
ethyl ({(3R)-3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]pyrrolidin-1-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(piperidin-4-yl)acetamide;
ethyl ({4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]azetidine-1-carboxylate;
N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}-N-[2-(trifluoromethyl)benzyl]acetamide;
N-(2-chloro-4-fluorobenzyl)-N-(cyclopropylmethyl)-2-{(1R)-5-[(methylcarbonyl)amino]-2′,4′-dioxo-2,3-dihydro-3′H-spiro[indene-1,5′-[1,3]ozazolidin]-3′-yl}acetamide;
N-(4-fluorobenzyl)-N-(3-methoxyphenyl)-2-{(1R)-5-[(methylcarbamoyl)amino]-2′,4′-dioxo-2,3-dihydro-3′H-spiro[indene-[1,3]ozazolidin]-3′-yl}acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-(pyridin-4-yl)acetamide;
N-(4-fluorobenzyl)-2-(6-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl)-N-[(2S)-1,1,1-trifluoropropan-yl]acetamide;
methyl (1S,3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(tetrahydro-2H-pyran-4-yl)acetamide;
N-(1,1-dioxidotetrahydro-2H-thiopyran-4-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2,2-difluorocyclopentyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-cyclohexyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-[(1S)-1-cyclopropyl-2,2,2-trifluoroethyl]-N-(4-fluorobenzyl)-2-[7-(1-methyl-1H-pyrazol-4-yl)-2′,4′-dioxo-1H,3′H-spiro[isochromene-4,5′-[1,3]ozazolidin]-3′-yl]acetamide;
2-(7-amino-2′,4′-dioxo-1H,3′H-spiro[isochromene-4,5′-[1,3]ozazolidin]-3′-yl)-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{7′-[(methylcarbamoyl)amino]-2′,4′-dioxo-1H,3′H-spiro[isochromene-4,5′-[1,3]ozazolidin]-3′-yl}acetamide;
N-(4-fluorobenzyl)-2-{7′-[(methylcarbamoyl)amino]-2′,4′-dioxo-1H,3′H-spiro[isochromene-4,5′-[1,3]ozazolidin]-3′-yl}-N-[(2S)-1,1,1-trifluoropropan-2-yl]acetamide;
N-(4-fluorobenzyl)-2-[7-(1-methyl-1H-pyrazol-4-yl)-2′,4′-dioxo-1H,3′H-spiro[isochromene-4,5′-[1,3]ozazolidin]-3′-yl]-N-[(2S)-1,1,1-trifluoropropan-2-yl]acetamide;
N-(4-fluorobenzyl)-2-(7-{1-[2-(methylamino)-2-oxoethyl]-1H-pyrazol-4-yl}-2′,4′-dioxo-1H,3′H-spiro[isochromene-4,5′-[1,3]ozazolidin]-3′-yl)-N-[(2S)-1,1,1-trifluoropropan-2-yl]acetamide;
N-(4-fluorobenzyl)-N-{1-[2-(methylamino)-2-oxoethyl]-1H-pyrazol-4-yl}-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[1-(cyanomethyl)-1H-pyrazol-4-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl 3-{1-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]ethyl}azetidine-1-carboxylate;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylic acid;
ethyl ({(3S)-3-[(4-fluorobenzyl) {[(1R)-5-{1-[2-(methylamino)-2-oxoethyl]-1H-pyrazol-4-yl}-2′,4′-dioxo-2,3-dihydro-3′H-spiro[indene-,5′-[1,3]ozazolidin]-3′-yl]acetyl}amino]pyrrolidin-1-yl}sulfonyl)carbamate;
ethyl ({(3S)-3-[{[(1R)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}(4-fluorobenzyl)amino]pyrrolidin-1-yl}sulfonyl)carbamate;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}acetate;
ethyl ({(3R)-3-[(4-fluorobenzyl) {[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]piperidin-1-yl}sulfonyl)carbamate;
ethyl ({(3R)-3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]piperidin-1-yl}sulfonyl)carbamate;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}acetic acid;
N-(4-fluorobenzyl)-2-{(4S)-7-[(methylcarbamoyl)amino]-2′,4′-dioxo-1H,3′H-spiro[isochromene-4,5′-[1,3]ozazolidin]-3′-yl}-N-[(2 S)-1,1,1-trifluoropropan-2-yl]acetamide;
N-(4-fluorobenzyl)-2-{(4R)-7-[(methylcarbamoyl)amino]-2′,4′-dioxo-1H,3′H-spiro[isochromene-4,5′-[1,3]ozazolidin]-3′-yl}-N-[(2 S)-1,1,1-trifluoropropan-2-yl]acetamide;
tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
tert-butyl (3 S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-piperidin-3-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3S)-piperidin-3-yl]acetamide;
N-[4-amino-3-(hydroxymethyl)butan-2-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({(3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzamide;
tert-butyl 2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(pyrrolidin-2-ylmethyl)acetamide;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexylidene}acetate;
N-ethyl-4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxamide;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexylidene}acetic acid;
N-(4-fluorobenzyl)-2-[(1R)-5-(oxetan-3-ylamino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzamide;
N-[(1-acetylpyrrolidin-2-yl)methyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{[1-(methylsulfonyl)pyrrolidin-2-yl]methyl}acetamide;
2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}-N-methylpyrrolidine-1-carboxamide;
N-(3,4-difluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{[1-(methylsulfamoyl)pyrrolidin-2-yl]methyl}acetamide;
N-(cyclopropylmethyl)-N-(2,3-dihydro-1H-inden-1-yl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexyl}acetic acid;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-3-methylazetidin-1-yl}sulfonyl)carbamate;
N-(3-cyanophenyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
tert-butyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}carbamate;
N-(3-aminocyclobutyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[3-(acetylamino)cyclobutyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{3-[(methylsulfonyl)amino]cyclobutyl}acetamide;
2,2′-({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}imino)diacetic acid;
N-{3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}glycine;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxylate;
methyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}carbamate;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}sulfamoyl)carbamate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxylic acid;
ethyl ({4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]piperidin-1-yl}sulfonyl)carbamate;
2-[(1 S,3S)-5-bromo-3-hydroxy-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(5-chloro-4-cyano-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
benzyl 3-{[(4-fluorobenzyl)({(1R)-5-[(methylcarbamoyl)amino]-2′,4′-dioxo-2,3-dihydro-3′H-spiro[indene-,5′-[1,3]ozazolidin]-3′-yl}acetyl)amino]methyl}azetidine-1-carboxylate;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoate;
N-(4-fluorobenzyl)-2-[(1 S,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
ethyl ({3,3-difluoro-4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
ethyl ({3,3-difluoro-4-[(4-fluorobenzyl){[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
N-(4-fluorobenzyl)-2-[(1S)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1S)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-N-(3-methoxycyclobutyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-N-methylcyclobutanecarboxamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxamide;
2-(5-amino-4-cyano-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetic acid;
(2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidin-1-yl)acetic acid;
methyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetate;
{3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetic acid;
tert-butyl {4-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]cyclohexylidene}acetate;
tert-butyl {4-[(4-fluorobenzyl) {[(1S)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetate;
{4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetic acid;
N-(4-fluorobenzyl)-2-[(1 S,3R)-3-fluoro-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(azetidin-3-ylmethyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
{4-[{[(1R)-2′,4′-dioxo-2,3-dihydro-3′H-spiro[indene-1,5′-[1,3]ozazolidin]-3′-yl]acetyl}(4-fluorobenzyl)amino]cyclohexylidene}acetic acid;
tert-butyl {trans-4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexyl}carbamate;
N-(trans-4-aminocyclohexyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{trans-4-[(methylsulfonyl)amino]cyclohexyl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(trans-4-{[(trifluoromethyl)sulfonyl]amino}cyclohexyl)acetamide;
tert-butyl 6-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-2-azaspiro[3.3]heptane-2-carboxylate;
N-(2-azaspiro[3.3]hept-6-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(1 S,3R)-5-bromo-3-hydroxy-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1-acetylazetidin-3-yl)methyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-[(2S)-1,1,1-trifluoropropan-2-yl]-2-(2′,3,4′-trioxo-1,2,3,4, 8,9-hexahydro-3′H-spiro[cyclopenta[f]quinazoline-7,5′-[1,3]ozazolidin]-3′-yl)acetamide;
ethyl [(3-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}azetidin-1-yl)sulfonyl]carbamate;
ethyl [(2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidin-1-yl)sulfonyl]carbamate;
ethyl ({6-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-2-azaspiro[3.3]hept-2-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(3-{[(trifluoromethyl)sulfonyl]amino}cyclobutyl)acetamide;
tert-butyl 3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]benzoate;
tert-butyl 3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]benzoate;
3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]benzoic acid;
N-(4-fluorobenzyl)-2-[(1S)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(azetidin-3-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]azetidin-1-yl}sulfonyl)carbamate;
2-[(1S)-5-bromo-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1 S,3R)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-[(1S)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-D-valinamide;
N-benzyl-N-[(1S)-1-cyclopropylethyl]-2-[2′,4′-dioxo-5-(2H-tetrazol-5-yl)-2,3-dihydro-3′H-spiro[indene-1,5′-[1,3]ozazolidin]-3′-yl]acetamide;
2,2′-({[3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]carbonyl}imino)diacetic acid;
N-(4-fluorobenzyl)-2-[(1 S,3R)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1 S,3R)-5-[1-(2-amino-2-oxoethyl)-1H-pyrazol-4-yl]-3-hydroxy-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1S)-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
methyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanoate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanoic acid;
2-{(1S)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R,3R)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R,3R)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1 S,3S)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1S)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1 S,3R)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyridin-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(pyridin-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(pyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(6-oxo-1,6-dihydropyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(6-oxo-1,6-dihydropyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-phenyl-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-phenyl-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclohexyl-N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-(tetrahydro-2H-pyran-4-yl)acetamide;
3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclobutanecarboxylic acid;
4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexanecarboxylic acid;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-{trans-4-[(methylsulfonyl)amino]cyclohexyl}acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-(3-{[(trifluoromethyl)sulfonyl]amino}cyclobutyl)acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-[1-(methylsulfonyl)piperidin-4-yl]acetamide;
2-[(1R,3S)-5-bromo-3-hydroxy-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R,3S)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2,2′-({3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclobutyl}imino)diacetic acid;
N-(4-fluorobenzyl)-2-[(1 S,3R)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(1,2-oxazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1S)-5-(1,2-oxazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1 S,3R)-3-fluoro-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-[(1R,3S)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R,3S)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
{4-[(4-fluorobenzyl) {[(1 S,3R)-3-fluoro-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetic acid;
N-(4-fluorobenzyl)-2-[(1R,3R)-3-fluoro-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R,3R)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide; and pharmaceutically acceptable salts thereof.

Still another embodiment pertains to compounds of Formula (Xb), selected from the group consisting of:
N-benzyl-N-(1-cyclobutylethyl)-2-(2,5-dioxo-2′,3′-dihydrospiro[imidazolidine-4,1′-indene]-1-yl)acetamide;
N-benzyl-N-(1-cyclopropylethyl)-2-(2,5-dioxo-3′,4′-dihydro-2′H-spiro[imidazolidine-4,1′-naphthalen]-1-yl)acetamide;
N-benzyl-N-(1-cyclopropylethyl)-2-(2′,5′-dioxospiro[chroman-4,4′-imidazolidine]-1′-yl)acetamide;
N-benzyl-N-(1-cyclopropylethyl)-2-(2,2′,5-trioxospiro[imidazolidine-4,3′-indoline]-1-yl)acetamide;
N-(4-bromobenzyl)-N-(1-cyclopropylethyl)-2-(2,5-dioxo-2′,3′-dihydrospiro[imidazolidine-4,1′-indene]-1-yl)acetamide;
N-(1-cyclopropylethyl)-2-(2,5-dioxo-2′,3′-dihydrospiro[imidazolidine-4,1′-indene]-1-yl)-N-(furan-2-ylmethyl)acetamide;
N-benzyl-N-(1-cyclopropylethyl)-2-(1′-methyl-2,2′,5-trioxospiro[imidazolidine-4,3′-indoline]-1-yl)acetamide;
N-(1-cyclopropylethyl)-2-(2,5-dioxo-2′,3′-dihydrospiro[imidazolidine-4,1′-indene]-1-yl)-N-(thiophen-2-ylmethyl)acetamide;
N-benzyl-N-(1-cyclopropylethyl)-2-(3-methyl-2,5-dioxo-2′,3′-dihydrospiro[imidazolidine-4,1′-indene]-1-yl)acetamide;
N-(1-cyclopropylethyl)-2-(2,5-dioxo-2′,3′-dihydrospiro[imidazolidine-4,1′-indene]-1-yl)-N-(4-fluorobenzyl)acetamide;
N-(1-cyclopropylethyl)-2-(2,5-dioxo-2′,3′-dihydrospiro[imidazolidine-4,1′-indene]-1-yl)-N-(2-fluorobenzyl)acetamide;
N-(1-cyclopropylethyl)-2-(2,5-dioxo-2′,3′-dihydrospiro[imidazolidine-4,1′-indene]-1-yl)-N-(3-fluorobenzyl)acetamide;
N-benzyl-2-(5′-bromo-2,5-dioxo-2′,3′-dihydrospiro[imidazolidine-4,1′-indene]-1-yl)-N-(1-cyclopropylethyl)acetamide;
2-(5′-bromo-2,5-dioxo-2′,3′-dihydrospiro[imidazolidine-4,1′-indene]-1-yl)-N-(1-cyclopropylethyl)-N-(furan-2-ylmethyl)acetamide;
N-benzyl-N-(1-cyclopropylethyl)-2-(5′-fluoro-2,5-dioxo-2′,3′-dihydrospiro[imidazolidine-4,1′-indene]-1-yl)acetamide;
N-benzyl-2-(4′-bromo-2,5-dioxo-2′,3′-dihydrospiro[imidazolidine-4,1′-indene]-1-yl)-N-(1-cyclopropylethyl)acetamide;
N-(1-cyclopropylethyl)-2-(2,5-dioxo-2′,3′-dihydrospiro[imidazolidine-4,1′-indene]-1-yl)-N-(pyridin-3-ylmethyl)acetamide;
N-(1-cyclopropylethyl)-2-(2,5-dioxo-2′,3′-dihydrospiro[imidazolidine-4,1′-indene]-1-yl)-N-(pyridin-2-ylmethyl)acetamide;
N-benzyl-N-(1-cyclopropylethyl)-2-(5′-methoxy-2,5-dioxo-2′,3′-dihydrospiro[imidazolidine-4,1′-indene]-1-yl)acetamide;
N-benzyl-N-(1-cyclopropylethyl)-2-(4′-methoxy-2,5-dioxo-2′,3′-dihydrospiro[imidazolidine-4,1′-indene]-1-yl)acetamide;
N-benzyl-N-(1-cyclopropylethyl)-2-(2′,5′-dioxo-6,7-dihydrospiro[cyclopenta[b]pyridine-5,4′-imidazolidine]-1′-yl]acetamide;
N-benzyl-2-(5′-cyano-2,5-dioxo-2′,3′-dihydrospiro[imidazolidine-4,1′-indene]-1-yl)-N-(1-cyclopropylethyl)acetamide;
N-(1-cyclopropylethyl)-2-(2,5-dioxo-2′,3′-dihydrospiro[imidazolidine-4,1′-indene]-1-yl)-N-(pyridin-4-ylmethyl)acetamide;
N-benzyl-2-(4′-chloro-2,5-dioxo-2′,3′-dihydrospiro[imidazolidine-4,1′-indene]-1-yl)-N-(1-cyclopropylethyl)acetamide;
N-benzyl-2-(2,5-dioxo-2′,3′-dihydrospiro[imidazolidine-4,1′-indene]-1-yl)-N-isopropylacetamide;
N-benzyl-N-sec-butyl-2-(2,5-dioxo-2′,3′-dihydrospiro[imidazolidine-4,1′-indene]-1-yl)acetamide;
N-(1-cyclobutylethyl)-2-(2,5-dioxo-2′,3′-dihydrospiro[imidazolidine-4,1′-indene]-1-yl)-N-(4-fluorobenzyl)acetamide;
N-benzyl-2-(4′-bromo-2,5-dioxo-2′,3′-dihydrospiro[imidazolidine-4,1′-indene]-1-yl)-N—((R)-1-cyclobutylethyl)acetamide;
2-(4′-bromo-2,5-dioxo-2′,3′-dihydrospiro[imidazolidine-4,1′-indene]-1-yl)-N-(1-cyclopropylethyl)-N-(4-fluorobenzyl)acetamide;
N-benzyl-2-(5′-bromo-2,5-dioxo-2′,3′-dihydrospiro[imidazolidine-4,1′-indene]-1-yl)-N-(1-cyclobutylethyl)acetamide;
2-(5′-bromo-2,5-dioxo-2′,3′-dihydrospiro[imidazolidine-4,1′-indene]-1-yl)-N-(1-cyclopropylethyl)-N-(4-fluorobenzyl)acetamide;
1-(2-(benzyl(1-cyclopropylethyl)amino)-2-oxoethyl)-2,5-dioxo-2′,3′-dihydrospiro[imidazolidine-4,1′-indene]-5′-carboxamide;
N-benzyl-2-(4′-cyano-2,5-dioxo-2′,3′-dihydrospiro[imidazolidine-4,1-indene]-1-yl)-N-(1-cyclopropylethyl)acetamide;
1-(2-(benzyl(1-cyclopropylethyl)amino)-2-oxoethyl)-2,5-dioxo-2′,3′-dihydrospiro[imidazolidine-4,1′-indene]-4′-carboxamide;
N-(1-cyclopropylethyl)-2-(2,5-dioxo-2′,3′-dihydrospiro[imidazolidine-4,1′-indene]-1-yl)-N-(naphthalen-2-ylmethyl)acetamide;
N-((5-bromofuran-2-yl)methyl)-N-(1-cyclopropylethyl)-2-(2,5-dioxo-2′,3′-dihydrospiro[imidazolidine-4,1′-indene]-1-yl)acetamide;
N-benzyl-N—((S)-1-cyclobutylethyl)-2-((S)-2,5-dioxo-2′,3′-dihydrospiro[imidazolidine-4,1′-indene]-1-yl)acetamide;
N-benzyl-N—((R)-1-cyclobutylethyl)-2-((S)-2,5-dioxo-2′,3′-dihydrospiro[imidazolidine-4,1′-indene]-1-yl)acetamide;
N-benzyl-N-(1-cyclopropylethyl)-2-(2′,5′-dioxo-5,6-dihydrospiro[cyclopenta[b]thiophene-4,4′-imidazolidine]-1′-yl)acetamide;
N-benzyl-N-(1-cyclopropylethyl)-2-(2′,5′-dioxo-4,5-dihydrospiro[cyclopenta[b]thiophene-6,4′-imidazolidine]-1′-yl)acetamide;
N-((1H-indol-5-yl)methyl)-N-(1-cyclopropylethyl)-2-(2,5-dioxo-2′,3′-dihydrospiro[imidazolidine-4,1′-indene]-1-yl)acetamide;
N-benzyl-2-(2,5-dioxo-2′,3′-dihydrospiro[imidazolidine-4,1′-indene]-1-yl)-N-(pentan-3-yl)acetamide;
N-benzyl-N-(1-cyclopropylethyl)-2-(3′-methoxy-2,5-dioxo-2′,3′-dihydrospiro[imidazolidine-4,1′-indene]-1-yl)acetamide;
N-benzyl-N-(1-cyclopropylethyl)-2-(5′-(methylthio)-2,5-dioxo-2′,3′-dihydrospiro[imidazolidine-4,1′-indene]-1-yl)acetamide;
N-benzyl-2-(7′-chloro-2,5-dioxo-2′,3′-dihydrospiro[imidazolidine-4,1′-indene]-1-yl)-N-(1-cyclobutylethyl)acetamide;
N-benzyl-N-(1-cyclopropylethyl)-2-(5′-(hydroxymethyl)-2,5-dioxo-2′,3′-dihydrospiro[imidazolidine-4,1′-indene]-1-yl)acetamide;
N-benzyl-N-(1-cyclopropylethyl)-2-(5′-((difluoromethoxy)methyl)-2,5-dioxo-2′,3′-dihydrospiro[imidazolidine-4,1′-indene]-1-yl)acetamide;
N-benzyl-2-(5′-cyclopropyl-2,5-dioxo-2′,3′-dihydrospiro[imidazolidine-4,1′-indene]-1-yl)-N-(1-cyclopropylethyl)acetamide;
N-benzyl-N-(1-cyclobutylethyl)-2-(3′-methyl-2,5-dioxo-2′,3′-dihydrospiro[imidazolidine-4,1′-indene]-1-yl)acetamide;
N-benzyl-N-(1-cyclobutylethyl)-2-(5′-(difluoromethoxy)-2,5-dioxo-2′,3′-dihydrospiro[imidazolidine-4,1′-indene]-1-yl)acetamide;
N-benzyl-N-(1-cyclopropylethyl)-2-(4′-methyl-2,5-dioxo-2′,3′-dihydrospiro[imidazolidine-4,1′-indene]-1-yl)acetamide;
2-(5′-acetamido-2,5-dioxo-2′,3′-dihydrospiro[imidazolidine-4,1′-indene]-1-yl)-N-benzyl-N-(1-cyclopropylethyl)acetamide;
N-benzyl-N-(1-cyclopropylethyl)-2-(5′-methyl-2,5-dioxo-2′,3′-dihydrospiro[imidazolidine-4,1′-indene]-1-yl)acetamide;
1-(2-(benzyl((S)-1-cyclopropylethyl)amino)-2-oxoethyl)-N-(cyanomethyl)-2,5-dioxo-2′,3′-dihydrospiro[imidazolidine-4,1′-indene]-5′-carboxamide;
N-benzyl-N-(1-cyclopropylethyl)-2-(5′-ethynyl-2,5-dioxo-2′,3′-dihydrospiro[imidazolidine-4,1′-indene]-1-yl)acetamide;
N-benzyl-N-(1-cyclopropylethyl)-2-(5′-(3-hydroxyprop-1-ynyl)-2,5-dioxo-2′,3′-dihydrospiro[imidazolidine-4,1′-indene]-1-yl)acetamide;
N-benzyl-N-(1-cyclobutylethyl)-2-(6′-methoxy-2,5-dioxo-2′,3′-dihydrospiro[imidazolidine-4,1′-indene]-1-yl)acetamide;
N-benzyl-N-(dicyclopropylmethyl)-2-(2,5-dioxo-2′,3′-dihydrospiro[imidazolidine-4,1′-indene]-1-yl)acetamide;
N-benzyl-2-(6′-chloro-2,5-dioxo-2′,3′-dihydrospiro[imidazolidine-4,1′-indene]-1-yl)-N-(1-cyclobutylethyl)acetamide;
N-benzyl-N-(1-cyclopropylethyl)-2-(2,5-dioxo-5′-(1H-pyrazol-5-yl)-2′,3′-dihydrospiro[imidazolidine-4,1′-indene]-1-yl)acetamide;
N-(4-chlorobenzyl)-N—((S)-1-cyclobutylethyl)-2-((S)-2,5-dioxo-2′,3′-dihydrospiro[imidazolidine-4,1′-indene]-1-yl)acetamide;
N-benzyl-N-(1-cyclopropylethyl)-2-(2,5-dioxo-5′-(1H-1,2,3-triazol-4-yl)-2′,3′-dihydrospiro[imidazolidine-4,1′-indene]-1-yl)acetamide;
N-benzyl-N—((S)-1-cyclobutylethyl)-2-((3′R)-3′-hydroxy-2,5-dioxo-2′,3′-dihydrospiro[imidazolidine-4,1′-indene]-1-yl)acetamide;
N-benzyl-N—((S)-1-cyclobutylethyl)-2-((3′S)-3′-hydroxy-2,5-dioxo-2′,3′-dihydrospiro[imidazolidine-4,1′-indene]-1-yl)acetamide;
N-(4-bromobenzyl)-N—((S)-1-cyclobutylethyl)-2-((S)-2,5-dioxo-2′,3′-dihydrospiro[imidazolidine-4,1′-indene]-1-yl)acetamide;
N-benzyl-N-(1-cyclopropylethyl)-2-(2,2′,5′-trioxo-1,2,6,7-tetrahydrospiro[cyclopenta[b]pyridine-5,4′-imidazolidine]-1′-yl)acetamide;
2-(5′-(1H-imidazol-2-yl)-2,5-dioxo-2′,3′-dihydrospiro[imidazolidine-4,1′-indene]-1-yl)-N-benzyl-N-(1-cyclopropylethyl)acetamide;
N—((S)-1-cyclobutylethyl)-2-((S)-2,5-dioxo-2′,3′-dihydrospiro[imidazolidine-4,1′-indene]-1-yl)-N-(4-(methylthio)benzyl)acetamide;
N-benzyl-N-(1-cyclopropylethyl)-2-(2,5-dioxo-5′-(1H-1,2,3-triazol-1-yl)-2′,3′-dihydrospiro[imidazolidine-4,1′-indene]-1-yl)acetamide;
N-benzyl-N-(1-cyclopropylethyl)-2-(2,5-dioxo-5′-(2H-1,2,3-triazol-2-yl)-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(4-fluorobenzyl)acetamide;
N-(4-cyanobenzyl)-N—((S)-1-cyclobutylethyl)-2-((S)-2,5-dioxo-2′,3′-dihydrospiro[imidazolidine-4,1′-indene]-1-yl)acetamide;
4-((N—((S)-1-cyclobutylethyl)-2-((S)-2,5-dioxo-2′,3′-dihydrospiro[imidazolidine-4,1′-indene]-1-yl)acetamido)methyl)benzamide;
N-benzyl-N-(1-cyclopropylethyl)-2-(5′-(methoxymethyl)-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(4-methylbenzyl)acetamide;
N—((S)-1-cyclobutylethyl)-2-((S)-2,5-dioxo-2′,3′-dihydrospiro[imidazolidine-4,1′-indene]-1-yl)-N-(4-methoxybenzyl)acetamide;
N—((S)-1-cyclobutylethyl)-2-((S)-2,5-dioxo-2′,3′-dihydrospiro[imidazolidine-4,1′-indene]-1-yl)-N-(3-methylbenzyl)acetamide;
N—((S)-1-cyclobutylethyl)-2-((S)-2,5-dioxo-2′,3′-dihydrospiro[imidazolidine-4,1′-indene]-1-yl)-N-(2-methylbenzyl)acetamide;
N-((1H-1,2,3-triazol-4-yl)methyl)-N—((S)-1-cyclobutylethyl)-2-((S)-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(4-(methylsulfonyl)benzyl)acetamide;
N-benzyl-N—((S)-1-cyclopropylethyl)-2-(2,3′,5-trioxo-2′,3′-dihydrospiro[imidazolidine-4,1′-indene]-1-yl)acetamide;
N-benzyl-2-(5′-(2-cyanoacetamido)-2,5-dioxo-2′,3′-dihydrospiro[imidazolidine-4,1′-indene]-1-yl)-N—((S)-1-cyclopropylethyl)acetamide;
methyl 1-(2-(benzyl((S)-1-cyclopropylethyl)amino)-2-oxoethyl)-2,5-dioxo-2′,3′-dihydrospiro[imidazolidine-4,1′-indene]-5′-ylcarbamate;
N-benzyl-N—((S)-1-cyclopropylethyl)-2-(7′-fluoro-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(1-phenylethyl)acetamide;
N-benzyl-2-(7′-cyano-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(5′-(3-methylureido)-2,5-dioxo-2′,3′-dihydrospiro[imidazolidine-4,1′-indene]-1-yl)acetamide;
N-benzyl-N—((S)-1-cyclopropylethyl)-2-(5′-(3-methyl-3-(methylcarbamoyl)ureido)-2,5-dioxo-2′,3′-dihydrospiro[imidazolidine-4,1′-indene]-1-yl)acetamide;
N—((S)-1-cyclopropylethyl)-2-((S)-2,5-dioxo-2′,3′-dihydrospiro[imidazolidine-4,1′-indene]-1-yl)-N-(4-ethynylbenzyl)acetamide;
N—((S)-3-cyclobutylethyl)-2-((S)-2,5-dioxo-2′,3′-dihydrospiro[imidazolidine-4,1′-indene]-1-yl)-N-((1-methyl-1H-1,2,3-triazol-4-yl)methyl)acetamide;
N-((1-(cyanomethyl)-1H-1,2,3-triazol-4-yl)methyl)-N—((S)-1-cyclobutylethyl)-2-((S)-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(4-ureidobenzyl)acetamide;
N-benzyl-N—((S)-1-cyclopropylethyl)-2-(5′-(3-hydroxy-3-methylbut-1-ynyl)-2,5-dioxo-2′,3′-dihydrospiro[imidazolidine-4,1′-indene]-1-yl)acetamide;
N-((1-(2-amino-2-oxoethyl)-1H-1,2,3-triazol-4-yl)methyl)-N—((S)-1-cyclobutylethyl)-2-((S)-2,5-dioxo-2′,3′-dihydrospiro[imidazolidine-4,1′-indene]-1-yl)acetamide;
(S)—N-benzyl-N-(1-cyclopropylethyl)-2-(2,5-dioxo-5′-(6-oxo-1,6-dihydropyridin-3-yl)-2′,3′-dihydrospiro[imidazolidine-4,1′-indene]-1-yl)acetamide;
N-benzyl-N—((S)-1-cyclopropylethyl)-2-((S)-2,5-dioxo-5′-(1H-pyrazol-3-yl)-2′,3′-dihydrospiro[imidazolidine-4,1′-indene]-1-yl)acetamide;
N-benzyl-N—((S)-1-cyclopropylethyl)-2-(5′-(methylsulfonamido)-2,5-dioxo-2′,3′-dihydrospiro[imidazolidine-4,1′-indene]-1-yl)acetamide;
N-benzyl-2-(5′-(2-cyanoacetamido)-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(2,5-dioxo-5′-(2H-tetrazol-5-yl)-2′,3′-dihydrospiro[imidazolidine-4,1′-indene]-1-yl)acetamide;
(S)—N-benzyl-N-(1-cyclopropylethyl)-2-(3′-(hydroxyimino)-2,5-dioxo-2′,3′-dihydrospiro[imidazolidine-4,1′-indene]-1-yl)acetamide;
(S)—N-benzyl-N-(1-cyclopropylethyl)-2-(3′-(methoxyamino)-2,5-dioxo-2′,3′-dihydrospiro[imidazolidine-4,1′-indene]-1-yl)acetamide;
N-benzyl-N—((S)-1-cyclopropylethyl)-2-((S)-2,5-dioxo-5′-(6-oxo-1,6-dihydropyridin-3-yl)-2′,3′-dihydrospiro[imidazolidine-4,1′-indene]-1-yl)acetamide;
N-benzyl-N—((S)-1-cyclopropylethyl)-2-((S)-5′-(3-hydroxyprop-1-ynyl)-2,5-dioxo-2′,3′-dihydrospiro[imidazolidine-4,1′-indene]-1-yl)acetamide;
N-benzyl-N—((S)-1-cyclopropylethyl)-2-((S)-2,5-dioxo-5′-(2H-1,2,3-triazol-4-yl)-2′,3′-dihydrospiro[imidazolidine-4,1′-indene]-1-yl)acetamide;
N-benzyl-N—((S)-1-cyclopropylethyl)-2-(5′-(3-hydroxypyrrolidin-1-yl)-2,5-dioxo-2′,3′-dihydrospiro[imidazolidine-4,1′-indene]-1-yl)acetamide;
N-benzyl-N—((S)-1-cyclopropylethyl)-2-((S)-5′-(3-hydroxy-3-methylbut-1-ynyl)-2,5-dioxo-2′,3′-dihydrospiro[imidazolidine-4,1′-indene]-1-yl)acetamide;
N-benzyl-2-((S)-5′-cyano-2,5-dioxo-2′,3′-dihydrospiro[imidazolidine-4,1′-indene]-1-yl)-N—((S)-1-cyclopropylethyl)acetamide;
1-(2-(benzyl((S)-1-cyclopropylethyl)amino)-2-oxoethyl)-2,5-dioxo-2′,3′-dihydrospiro[imidazolidine-4,1′-indene]-3′-carboxylic acid;
N-benzyl-N—((S)-1-cyclopropylethyl)-2-((S)-5′-(3-methylureido)-2,5-dioxo-2′,3′-dihydrospiro[imidazolidine-4,1′-indene]-1-yl)acetamide;
N-benzyl-N—((S)-1-cyclopropylethyl)-2-(5′-(3-hydroxyazetidin-1-yl)-2,5-dioxo-2′,3′-dihydrospiro[imidazolidine-4,1′-indene]-1-yl)acetamide;
N-benzyl-2-((S)-5′-(2′-cyanoacetamido)-2,5-dioxo-2′,3′-dihydrospiro[imidazolidine-4, 1′-indene]-1-yl)-N—((S)-1-cyclopropylethyl)acetamide;
1-(2-(benzyl((S)-1-cyclopropylethyl)amino)-2-oxoethyl)-2,5-dioxo-2′,3′-dihydrospiro[imidazolidine-4,1′-indene]-3′-carboxamide;
N-benzyl-2-(3′-cyano-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(5′-(2,4-dioxo-3,4-dihydropyrimidin-1(2H)-yl)-2,5-dioxo-2′,3′-dihydrospiro[imidazolidine-4,1′-indene]-1-yl)acetamide;
N-(1-(2-(benzyl((S)-1-cyclopropylethyl)amino)-2-oxoethyl)-2,5-dioxo-2′,3′-dihydrospiro[imidazolidine-4,1′-indene]-5′-ylacrylamide;
N-benzyl-2-(5′-(2-cyano-1-hydroxyallyl)-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(5′-(2,4-dioxo-1,2,3,4-tetrahydropyrimidin-5-yl)-2,5-dioxo-2′,3′-dihydrospiro[imidazolidine-4,1′-indene]-1-yl)acetamide;
1-(2-(benzyl((S)-1-cyclopropylethyl)amino)-2-oxoethyl)-2,5-dioxo-2′,3′-dihydrospiro[imidazolidine-4,1′-indene]-5′-carboxylic acid;
(S)—N-(1-(2-(benzyl(1-cyclopropylethyl)amino)-2-oxoethyl)-2,5-dioxo-2′,3′-dihydrospiro[imidazolidine-4,1′-indene]-5′-yl)-3-oxobutanamide;
N-(1-(2-(benzyl((S)-1-cyclopropylethyl)amino)-2-oxoethyl)-2,5-dioxo-2′,3′-dihydrospiro[imidazolidine-4,1′-indene]-5′-yl)-2-cyano-3-oxobutanamide;
(E)-N-(1-(2-(benzyl((S)-1-cyclopropylethyl)amino)-2-oxoethyl)-2,5-dioxo-2′,3′-dihydrospiro[imidazolidine-4,1′-indene]-5′-yl)-4-(piperidin-1-yl)but-2-enamide;
N-benzyl-2-((S)-2,5-dioxo-2′,3′-dihydrospiro[imidazolidine-4,1′-indene]-1-yl)-N-(1-(oxetan-3-yl)ethyl)acetamide;
2-amino-N-((1′S)-1-((4S)-3-benzyl-4-cyclopropyl-2-oxopentyl)-2,5-dioxo-2′,3′-dihydrospiro[imidazolidine-4,1′-indene]-5′-yl)-2-methylpropan amide;
(S)-2-amino-N—((S)-1-(2-(benzyl((S)-1-cyclopropylethyl)amino)-2-oxoethyl)-2,5-dioxo-2′,3′-dihydrospiro[imidazolidine-4,1′-indene]-5′-ylpropanamide;
N-benzyl-N—((S)-1-cyclopropylethyl)-2-(5′-(2-hydroxy-5-oxo-2,5-dihydrofuran-2-yl)-2,5-dioxo-2′,3′-dihydrospiro[imidazolidine-4,1′-indene]-1-yl)acetamide;
5′-(azetidin-3-ylmethylamino)-1-((4S)-3-benzyl-4-cyclopropyl-2-oxopentyl)-2′,3′-dihydrospiro[imidazolidine-4,1′-indene]-2,5-dione;
1-amino-N—((S)-1-(2-(benzyl((S)-1-cyclopropylethyl)amino)-2-oxoethyl)-2,5-dioxo-2′,3′-dihydrospiro[imidazolidine-4,1′-indene]-5′-yl)cyclobutanecarboxamide;
N-benzyl-N—((S)-1-cyclopropylethyl)-2-(2,5-dioxo-5′-(thiazol-2-ylamino)-2′,3′-dihydrospiro[imidazolidine-4,1′-indene]-1-yl)acetamide;
(S)-2-(5′-(azetidin-3-ylamino)-2,5-dioxo-2′,3′-dihydrospiro[imidazolidine-4,1′-indene]-1-yl)-N-benzyl-N-(1-cyclopropylethyl)acetamide;
N-benzyl-N-(1-cyclopropylethyl)-2-(2,5-dioxo-1′,3′-dihydrospiro[imidazolidine-4,2′-indene]-1-yl)acetamide;
N-benzyl-N-(1-cyclopropylethyl)-2-(8′-methoxy-2,5-dioxo-3′,4′-dihydro-1′H-spiro[imidazolidine-4,2′-naphthalen]-1-yl)acetamide;
N-benzyl-N-(1-cyclobutylethyl)-2-(3′,3′-dimethyl-2,5-dioxo-2′,3′-dihydrospiro[imidazolidine-4,1′-indene]-1-yl)acetamide;
N-benzyl-2-[5′-(5-cyano-6-oxo-1,6-dihydropyridin-3-yl)-2,5-dioxo-2′,3′-dihydrospiro[imidazolidine-4,1′-indene]-1-yl]-N-(1-cyclopropylethyl)acetamide;
(2R)-2-amino-N-[(4S)-1-({benzyl[(1S)-1-cyclopropylethyl]carbamoyl}methyl)-2,5-dioxo-2′,3′-dihydrospiro[imidazolidine-4,1′-indene]-5′-yl]propanamide;
N-benzyl-N-[(1S)-1-cyclopropylethyl]-2-{5′-[(1H-imidazol-2-yl)amino]-2,5-dioxo-2′,3′-dihydro spiro[imidazolidine-4,1′-indene]-1-yl}acetamide;
1-amino-N-[(4S)-1-({benzyl[(1S)-1-cyclopropylethyl]carbamoyl}methyl)-2,5-dioxo-2′,3′-dihydrospiro[imidazolidine-4,1′-indene]-5′-yl]cyclopropane-1-carboxamide;
N-benzyl-N-[(1R)-1-cyclopropyl-2,2,2-trifluoroethyl]-2-[(4S)-2,5-dioxo-2′,3′-dihydrospiro[imidazolidine-4,1′-indene]-1-yl]acetamide;
N-benzyl-N-[(1S)-1-cyclopropylethyl]-2-{5′-[(oxetan-3-ylmethyl)amino]-2,5-dioxo-2′,3′-dihydrospiro[imidazolidine-4,1′-indene]-1-yl}acetamide;
N-benzyl-N-[(1S)-1-cyclopropylethyl]-2-{1,1,2′,5′-tetraoxo-2H-spiro[1λ⁶-benzothiophene-3,4′-imidazolidine]-1′-yl}acetamide;
2-[5′-(azetidin-3-yloxy)-2,5-dioxo-2′,3′-dihydrospiro[imidazolidine-4,1′-indene]-1-yl]-N-benzyl-N-[(1S)-1-cyclopropylethyl]acetamide;
N-[(2-amino-1,3-thiazol-5-yl)methyl]-N-[(1S)-1-cyclopropylethyl]-2-[(4S)-2,5-dioxo-2′,3′-dihydrospiro[imidazolidine-4,1′-indene]-1-yl]acetamide;
N-[(1S)-1-cyclopropylethyl]-N-{[2-(dimethylamino)-1,3-thiazol-5-yl]methyl}-2-[(4S)-2,5-dioxo-2′,3′-dihydrospiro[imidazolidine-4,1′-indene]-1-yl]acetamide;
N-[(1S)-1-cyclopropylethyl]-N-{[6-(dimethylamino)pyridin-3-yl]methyl}-2-[(4S)-2,5-dioxo-2′,3′-dihydrospiro[imidazolidine-4,1′-indene]-1-yl]acetamide;
N-[(2-amino-1,3-thiazol-4-yl)methyl]-N-[(1S)-1-cyclopropylethyl]-2-[(4S)-2,5-dioxo-2′,3′-dihydrospiro[imidazolidine-4,1′-indene]-1-yl]acetamide;
N-[(1S)-1-cyclopropylethyl]-N-[(4-fluorophenyl)methyl]-2-{1,1,2′,5′-tetraoxo-2H-spiro[1λ⁶-benzothiophene-3,4′-imidazolidine]-1′-yl}acetamide;
N-[(1S)-1-cyclopropylethyl]-2-[(4S)-2,5-dioxo-2′,3′-dihydrospiro[imidazolidine-4,1′-indene]-1-yl]-N-[(6-hydroxypyridin-3-yl)methyl]acetamide;
N-[(1S)-1-cyclopropylethyl]-2-[(4S)-2,5-dioxo-2′,3′-dihydrospiro[imidazolidine-4,1′-indene]-1-yl]-N-[(6-methoxypyridin-3-ylmethyl]acetamide;
N-[(1S)-1-cyclopropylethyl]-N-{[2-(dimethylamino)-1,3-thiazol-4-yl]methyl}-2-[(4S)-2,5-dioxo-2′,3′-dihydrospiro[imidazolidine-4,1′-indene]-1-yl]acetamide;
N-benzyl-N-[(1R)-1-cyclopropyl-2,2,2-trifluoroethyl]-2-{1,1,2′,5′-tetraoxo-2H-spiro[1λ⁶-benzothiophene-3,4′-imidazolidine]-1′-yl}acetamide;
N-[(6-amino-5-methoxypyridin-3-yl)methyl]-N-[(1S)-1-cyclopropylethyl]-2-[(4S)-2,5-dioxo-2′,3′-dihydrospiro[imidazolidine-4,1′-indene]-1-yl]acetamide;
N-[(6-aminopyridin-3-yl)methyl]-N-[(1S)-1-cyclopropylethyl]-2-[(4S)-2,5-dioxo-2′,3′-dihydrospiro[imidazolidine-4,1′-indene]-1-yl]acetamide;
N-benzyl-2-{6-bromo-1,1,2′,5′-tetraoxo-2H-spiro[1λ⁶-benzothiophene-3,4′-imidazolidine]-1′-yl}-N-[(1S)-1-cyclopropylethyl]acetamide;
N-benzyl-N-[(1S)-1-cyclopropylethyl]-2-[1,1,2′,5′-tetraoxo-6-(1H-pyrazol-3-yl)-2H-spiro[1λ⁶-benzothiophene-3; 4′-imidazolidine]-1′-yl]acetamide;
2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide;
2-(6-bromo-1,1-dioxido-2′,5′-dioxo-1′H-spiro[1-benzothiophene-3,4′-imidazolidin]-1′-yl)-N-[(1S)-1-cyclopropylethyl]-N-(4-fluorobenzyl)acetamide;
N-[(1S)-1-cyclopropylethyl]-2-[2,5-dioxo-5′-(6-oxo-1,6-dihydropyridin-3-yl)-1′,3′-dihydro-1H-spiro[imidazolidine-4,2′-inden]-1-yl]-N-(4-fluorobenzyl)acetamide;
2-(5′-amino-2,5-dioxo-1′,3′-dihydro-1H-spiro[imidazolidine-4,2′-inden]-1-yl)-N-[(1S)-1-cyclopropylethyl]-N-(4-fluorobenzyl)acetamide;
N-[(1S)-1-cyclopropylethyl]-2-[1,1-dioxido-2′,5′-dioxo-6-(6-oxo-1,6-dihydropyridin-3-yl)-1′H-spiro[1-benzothiophene-3,4′-imidazolidin]-1′-yl]-N-(4-fluorobenzyl)acetamide;
2-(6-amino-1,1-dioxido-2′,5′-dioxo-1′H-spiro[1-benzothiophene-3,4′-imidazolidin]-1-yl)-N-[(1S)-1-cyclopropylethyl]-N-(4-fluorobenzyl)acetamide;
N-[(1S)-1-cyclopropylethyl]-N-(4-fluorobenzyl)-2-{5′-[(methylcarbamoyl)amino]-2,5-dioxo-1′,3′-dihydro-1H-spiro[imidazolidine-4,2′-inden]-1-yl}acetamide;
2-[5′-(acetylamino)-2,5-dioxo-1′,3′-dihydro-1H-spiro[imidazolidine-4,2′-inden]-1-yl]-N-[(1S)-1-cyclopropylethyl]-N-(4-fluorobenzyl)acetamide;
N-[(1S)-1-cyclopropylethyl]-N-(4-fluorobenzyl)-2-{6-[(methylcarbamoyl)amino]-1,1-dioxido-2′,5′-dioxo-1′H-spiro[1-benzothiophene-3,4′-imidazolidin]-1′-yl}acetamide;
N-(4-fluorobenzyl)-2-(1′-methyl-2,5-dioxo-2′,3′-dihydro-1H,1′H-spiro[imidazolidine-4,4′-quinolin]-1-yl)-N-[(2S)-1,1,1-trifluoropropan-2-yl]acetamide;
N-[(1S)-1-cyclopropylethyl]-N-(4-fluorobenzyl)-2-(3′-hydroxy-2,5-dioxo-2′,3′-dihydro-1H-spiro[imidazolidine-4,1′-inden]-1-yl)acetamide;
N-(4-fluorobenzyl)-N-[(2S)-1,1,1-trifluoropropan-2-yl]-2-(2,3′,5-trioxo-2′,3′-dihydro-1H-spiro[imidazolidine-4,1′-inden]-1-yl)acetamide;
N-[(1S)-1-cyclopropylethyl]-N-(4-fluorobenzyl)-2-(1′-methyl-2,5-dioxo-2′,3′-dihydro-1H,1′H-spiro[imidazolidine-4,4′-quinolin]-1-yl)acetamide
N-(4-fluorobenzyl)-2-[(3′E)-3′-(hydroxyimino)-2,5-dioxo-2′,3′-dihydro-1H-spiro[imidazolidine-4,1′-indene]-1-yl]-N-[(2S)-1,1,1-trifluoropropan-2-yl]acetamide;
N-[(1S)-1-cyclopropylethyl]-N-(4-fluorobenzyl)-2-(2,3′,5-trioxo-2′,3′-dihydro-1H-spiro[imidazolidine-4,1′-inden]-1-yl)acetamide;
N-(4-fluorobenzyl)-2-(3′-hydroxy-2,5-dioxo-2′,3′-dihydro-1H-spiro[imidazolidine-4,1′-inden]-1-yl)-N-[(2S)-1,1,1-trifluoropropan-2-yl]acetamide;
2-[6-(acetylamino)-1,1-dioxido-2′,5′-dioxo-1′H-spiro[1-benzothiophene-3,4′-imidazolidin]-1′-yl]-N-[(1S)-1-cyclopropylethyl]-N-(4-fluorobenzyl)acetamide;
2-(7′-bromo-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
N-(4-fluorobenzyl)-2-(3′-fluoro-2,5-dioxo-2′,3′-dihydro-1H-spiro[imidazolidine-4,1′-inden]-1-yl)-N-[(2S)-1,1,1-trifluoropropan-2-yl]acetamide;
2-(3′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[3′-(methylamino)-2,5-dioxo-2′,3′-dihydro-1H-spiro[imidazolidine-4,1′-inden]-1-yl]-N-[(2S)-1,1,1-trifluoropropan-2-yl]acetamide;
2-(7′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
2-(6-bromo-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide;
N-[(1S)-1-cyclopropylethyl]-N-(4-fluorobenzyl)-2-{7′-[(methylcarbamoyl)amino]-2,5-dioxo-3′,4′-dihydro-1H,1′H-spiro[imidazolidine-4,2′-naphthalen]-1-yl}acetamide;
2-[7′-(acetylamino)-2,5-dioxo-3′,4′-dihydro-1H,1′H-spiro[imidazolidine-4,2′-naphthalen]-1-yl]-N-[(1S)-1-cyclopropylethyl]-N-(4-fluorobenzyl)acetamide;
N-(4-fluorobenzyl)-2-(2′-methyl-2,5-dioxo-2′,3′-dihydro-1H-spiro[imidazolidine-4,1′-inden]-1-yl)-N-[(2S)-1,1,1-trifluoropropan-2-yl]acetamide;
N-[1-(2-{[(1S)-1-cyclopropylethyl](4-fluorobenzyl)amino}-2-oxoethyl)-2,5-dioxo-3′,4′-dihydro-1′H-spiro[imidazolidine-4,2′-naphthalen]-7′-yl]-5-oxo-D-prolinamide;
2-{7′-[1-(2-amino-2-oxoethyl)-1H-pyrazol-4-yl]-2,5-dioxo-3′,4′-dihydro-1H,1′H-spiro[imidazolidine-4,2′-naphthalen]-1-yl}-N-[(1S)-1-cyclopropylethyl]-N-(4-fluorobenzyl)acetamide;
2-(5′-bromo-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-N-[(2S)-1,1,1-trifluoropropan-2-yl]-2-(2,2′,5-trioxo-2′,3′-dihydro-1H,1′H-spiro[imidazolidine-4,4′-quinolin]-1-yl)acetamide;
2-(2; 5-dioxo-1H-spiro[imidazolidine-4,1′-inden]-1-yl)-N-(4-fluorobenzyl)-N-[(2S)-1,1,1-trifluoropropan-2-yl]acetamide;
2-{5′-[1-(2-amino-2-oxoethyl)-1H-pyrazol-4-yl]-2,3′,5-trioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-N-[(2S)-1,1,1-trifluoropropan-2-yl]-2-[2,3′,5-trioxo-5′-(6-oxo-1,6-dihydropyridin-3-yl)-2′,3′-dihydro-1H-spiro[imidazolidine-4,1′-inden]-1-yl]acetamide;
N-[(1S)-1-cyclopropylethyl]-2-[(4R)-2,5-dioxo-2′,3′-dihydro-1H-spiro[imidazoline-4,1′-inden]-1-yl]-N-(4-fluorobenzyl)acetamide;
N-[(1S)-1-cyclopropylethyl]-2-[(4S)-2,5-dioxo-2′,3′-dihydro-1H-spiro[imidazolidine-4,1′-inden]-1-yl]-N-(4-fluorobenzyl)acetamide;
2-(7′-bromo-1′-methyl-2,5-dioxo-2′,3′-dihydro-1H,1′H-spiro[imidazolidine-4,4′-quinolin]-1-yl)-N-[(1S)-1-cyclopropylethyl]-N-(4-fluorobenzyl)acetamide;
2-[5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-(4′-hydroxy-2,5-dioxo-3′,4′-dihydro-1H,2′H-spiro[imidazolidine-4,1′-naphthalen]-1-yl)-N-[(2S)-1,1,1-trifluoropropan-2-yl]acetamide;
2-(6-{1-[2-(dimethylamino)-2-oxoethyl]-1H-pyrazol-4-yl}-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide;
2-[5′-(acetylamino)-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[5′-(acetylamino)-2,5-dioxo-2′,3′-dihydro-1H-spiro[imidazolidine-4,1′-inden]-1-yl]-N-[(1S)-1-cyclopropylethyl]-N-(4-fluorobenzyl)acetamide;
N-(4-fluorobenzyl)-2-(5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-(3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
2-(6′-bromo-2,5-dioxo-3′,4′-dihydro-1H, 1′H-spiro[imidazolidine-4,2′-naphthalen]-1-yl)-N-[(1S)-1-cyclopropylethyl]-N-(4-fluorobenzyl)acetamide;
2-(6′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
2-(7′-amino-1′-methyl-2,5-dioxo-2′,3′-dihydro-1H,1′H-spiro[imidazolidine-4,4′-quinolin]-1-yl)-N-[(1S)-1-cyclopropylethyl]-N-(4-fluorobenzyl)acetamide;
N-[(1S)-1-cyclopropylethyl]-N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}acetamide;
N-[(1S)-1-cyclopropylethyl]-N-(4-fluorobenzyl)-2-{6′-[(methylcarbamoyl)amino]-2,5-dioxo-3′,4′-dihydro-1H,1H-spiro[imidazolidine-4,2′-naphthalen]-1-yl}acetamide;
2-[6′-(acetylamino)-2,5-dioxo-3′,4′-dihydro-1H,1′H-spiro[imidazolidine-4,2′-naphthalen]-1-yl]-N-[(1S)-1-cyclopropylethyl]-N-(4-fluorobenzyl)acetamide;
2-(6-amino-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide;
N-(4-fluorobenzyl)-2-(6-{1-[2-(methylamino)-2-oxoethyl]-1H-pyrazol-4-yl}-1,1-dioxido-2′,5′-dioxo-1′H-spiro[1-benzothiophene-3,4′-imidazolidin]-1′-yl)-N-[(2S)-1,1,1-trifluoropropan-2-yl]acetamide;
tert-butyl {4-[1-(2-{(4-fluorobenzyl)[(2S)-1,1,1-trifluoropropan-2-yl]amino}-2-oxoethyl)-2,3′,5-trioxo-2′,3′-dihydrospiro[imidazolidine-4,1′-inden]-5′-yl]-1H-pyrazol-1-yl}acetate;
N-(4-fluorobenzyl)-2-{3′-fluoro-5′-[1-(2-methylpropyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
{4-[1-(2-{(4-fluorobenzyl)[(2S)-1,1,1-trifluoropropan-2-yl]amino}-2-oxoethyl)-2,3′,5-trioxo-2′,3′-dihydrospiro[imidazolidine-4,1′-inden]-5′-yl]-1H-pyrazol-1-yl}acetic acid;
2-(5′-{1-[2-(dimethylamino)-2-oxoethyl]-1H-pyrazol-4-yl}-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-(5′-{1-[2-(3-hydroxyazetidin-1-yl)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-{6-[(methylcarbamoyl)amino]-1,1-dioxido-2′,5′-dioxo-1′H-spiro[1-benzothiophene-3,4′-imidazolidin]-1′-yl}-N-[(2S)-1,1,1-trifluoropropan-2-yl]acetamide;
2-(5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-{5′-[1-(2-amino-2-oxoethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-yl]acetamide;
2-{5′-[1-(2-amino-2-oxoethyl)-1H-pyrazol-4-yl]-3′-hydroxy-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-[(4S)-5′-{1-[2-(dimethylamino)-2-oxoethyl]-1H-pyrazol-4-yl}-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4S)-5′-{1-[2-(dimethylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-N-[(2S)-1,1,1-trifluoropropan-2-yl]-2-[(4R)-2,3′,5-trioxo-2′,3′-dihydro-1H-spiro[imidazolidine-4,1′-inden]-1-yl]acetamide;
N-(4-fluorobenzyl)-N-[(2S)-1,1,1-trifluoropropan-2-yl]-2-[(4S)-2,3′,5-trioxo-2′,3′-dihydro-1H-spiro[imidazolidine-4,1′-inden]-1-yl]acetamide;
N-(4-fluorobenzyl)-2-[5′-(1-methyl-1H-pyrazol-4-yl)-2,3′,5-trioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(7′-bromo-1′-methyl-2,5-dioxo-2′,3′-dihydro-1H,1′H-spiro[imidazolidine-4,4′-quinolin]-1-yl)-N-[(1S)-1-cyclopropyl-2,2,2-trifluoroethyl]-N-(4-fluorobenzyl)acetamide;
N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}-N-[(2S)-1,1,1-trifluoropropan-2-yl]acetamide;
N-(4-fluorobenzyl)-2-[(3′R,4R)-3′-hydroxy-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(3′S,4R)-3′-fluoro-2,5-dioxo-2′,3′-dihydro-1H-spiro[imidazolidine-4,1′-inden]-1-yl]-N-[(2S)-1,1,1-trifluoropropan-2-yl]acetamide;
2-(7′-bromo-2,2′,5-trioxo-2′,3′-dihydro-1H,1′H-spiro[imidazolidine-4,4′-quinolin]-1-yl)-N-(4-fluorobenzyl)-N-[(2S)-1,1,1-trifluoropropan-2-yl]acetamide;
N-(4-fluorobenzyl)-2-(7′-{1-[2-(methylamino)-2-oxoethyl]-1H-pyrazol-4-yl}-2,2′,5-trioxo-2′,3′-dihydro-1H,1′H-spiro[imidazolidine-4,4′-quinolin]-1-yl)-N-[(2S)-1,1,1-trifluoropropan-2-yl]acetamide;
2-{7′-[1-(2-amino-2-oxoethyl)-1H-pyrazol-4-yl]-2,2′,5-trioxo-2′,3′-dihydro-1H,1′H-spiro[imidazolidine-4,4′-quinolin]-1-yl}-N-(4-fluorobenzyl)-N-[(2S)-1,1,1-trifluoropropan-2-yl]acetamide;
N-[(1S)-1-cyclopropyl-2,2,2-trifluoroethyl]-N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}acetamide;
2-(5′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2,2,2-trifluoro-N-[1-(2-{(4-fluorobenzyl)[(2S)-1,1,1-trifluoropropan-2-yl]amino}-2-oxoethyl)-1′-methyl-2,5-dioxo-2′,3′-dihydro-1′H-spiro[imidazolidine-4,4′-quinolin]-7′-yl]acetamide;
N-[1-(2-{[(1S)-1-cyclopropylethyl](4-fluorobenzyl)amino}-2-oxoethyl)-1′-methyl-2,5-dioxo-2′,3′-dihydro-1′H-spiro[imidazolidine-4,4′-quinolin]-7′-yl]-2,2,2-trifluoroacetamide;
N-(4-fluorobenzyl)-2-[(3′S,4S)-3′-hydroxy-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[3′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(5′-bromo-3′-hydroxy-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[(3′R,4S)-3′-fluoro-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-{5′-[(methylcarbamoyl)amino]-2,3′,5-trioxo-2′,3′-dihydro-1H-spiro[imidazolidine-4,1′-inden]-1-yl}-N-[(2S)-1,1,1-trifluoropropan-2-yl]acetamide;
2-(5′-amino-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide;
2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{3′-hydroxy-5′-[1-(2-methyl)propyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(4S)-5′-bromo-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-(5′-bromo-3′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-{(4S)-5′-[1-(2-amino-2-oxoethyl)-1H-pyrazol-4-yl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-[(1S)-1-cyclopropylethyl]-N-(4-fluorobenzyl)-2-[1′-methyl-7′-(1-methyl-1H-pyrazol-4-yl)-2,5-dioxo-2′,3′-dihydro-1H,1′H-spiro[imidazolidine-4,4′-quinolin]-1-yl]acetamide;
N-(4-fluorobenzyl)-2-[7′-(1-methyl-1H-pyrazol-4-yl)-2,2′,5-trioxo-2′,3′-dihydro-1H,1′H-spiro[imidazolidine-4,4′-quinolin]-1-yl]-N-[(2S)-1,1,1-trifluoropropan-2-yl]acetamide;
N-(4-fluorobenzyl)-2-[(4S)-5′-(1-methyl-1H-pyrazol-4-yl)-2,3′,5-trioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(3′R,4S)-3′-hydroxy-2,5-dioxo-2′,3′-dihydro-1H-spiro[imidazolidin-4,1′-inden]-1-yl]-N-[(2S)-1,1,1-trifluoropropan-2-yl]acetamide;
N-(4-fluorobenzyl)-2-[(3′S,4S)-3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(4S)-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(3′S,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(4S)-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(3′S,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(3′R,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(3′S,4S)-5′-bromo-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(3′R,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(3′R,4S)-5′-bromo-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{(4S)-5′-[1-(2-amino-2-oxoethyl)-1H-pyrazol-4-yl]-2,3′,5-trioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-{(3′S,4S)-5′-[1-(2-amino-2-oxoethyl)-1H-pyrazol-4-yl]-3′-hydroxy-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-[(3′S,4S)-5′-{1-[2-(dimethylamino)-2-oxoethyl]-1H-pyrazol-4-yl}-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3′R,4S)-5′-{1-[2-(dimethylamino)-2-oxoethyl]-1H-pyrazol-4-yl}-3′-fluoro-2,5-dioxo-2′,3′-dihydro-1H-spiro[imidazolidine-4,1-inden]-1-yl]-N-(4-fluorobenzyl)-N-[(2S)-1,1,1-trifluoropropan-2-yl]acetamide;
N-(4-fluorobenzyl)-2-[(4S)-5′-{1-[2-(methylamino)-2-oxoethyl]-1H-pyrazol-4-yl}-2,5-dioxo-1H-spiro[imidazolidine-4,1′-inden]-1-yl]-N-[(2S)-1,1,1-trifluoropropan-2-yl]acetamide;
N-(3,4-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(2,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1′-trifluoropropan-2-yl]acetamide;
N-[(1S)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-[(1S)-1-cyclopropyl-2,2,2-trifluoroethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-[(1R)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-benzyl-N-[(1S)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-benzyl-N-[(1R)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide;
2-(4′-bromo-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[4′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-[(3′S,4S)-5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3S)-6-amino-1,1-dioxido-2′,5′-dioxo-1H-spiro[1-benzothiophene-3,4′-imidazolidin]-1′-yl]-N-(4-fluorobenzyl)-N-[(2S)-1,1,1-trifluoropropan-2-yl]acetamide;
2-[(3′R,4S)-5′-bromo-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-(5′-bromo-6′-fluoro-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-(5′-bromo-6′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-(6′-fluoro-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-(6′-fluoro-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-[4′-(1-methyl-1,2,3,6-tetrahydropyridin-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[(3′R,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(3R)-6-amino-1,1-dioxido-2′,5′-dioxo-1′H-spiro[1-benzothiophene-3,4′-imidazolidin]-1′-yl]-N-(4-fluorobenzyl)-N-[(2S)-1,1,1-trifluoropropan-2-yl]acetamide;
2-{2,5-dioxo-5′-[(2,2,2-trifluoroethyl)amino]-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-[(4S)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4R)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{(3′R,4S)-5′-[1-(2-amino-2-oxoethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-(5′-bromo-6′-fluoro-3′-hydroxy-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-(6′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
2-{5′-[1-(2-amino-2-oxoethyl)-1H-pyrazol-4-yl]-6′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-{6′-fluoro-5′-[1-(2-hydroxyethyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-[6′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(5′-bromo-3′,6′-difluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
tert-butyl 4-[1-(2-{(4-fluorobenzyl)[(2S)-1,1,1-trifluoropropan-2-yl]amino}-2-oxoethyl)-2,5-dioxo-2′,3′-dihydrospiro[imidazolidine-4,1′-inden]-4′-yl]-3,6-dihydropyridine-1(2H)-carboxylate;
2-[2,5-dioxo-4′-(1,2,3,6-tetrahydropyridin-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{5′-[1-(2-amino-2-oxoethyl)-1H-pyrazol-4-yl]-3′,6′-difluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-{4-[3′,6′-difluoro-1-(2-{(4-fluorobenzyl) [(2S)-1,1,1-trifluoropropan-2-yl]amino}-2-oxoethyl)-2,5-dioxo-2′,3′-dihydro-1H-spiro[imidazolidine-4,1′-inden]-5′-yl]-1H-pyrazol-1-yl}-N,N-dimethylacetamide;
2-[3′,6′-difluoro-5′-(1-methyl-1H-pyrazol-4-yl)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{3′,6′-difluoro-5′-[1-(2-hydroxyethyl)-H-pyrazol-4-yl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-[(4R)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-[(4S)-2,5-dioxo-5′-(1H-pyrazol-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{5′-[(1-methyl-1H-pyrazol-3-yl)amino]-2,5-dioxo-2′,3′-dihydro-1H-spiro[imidazolidine-4,1′-inden]-1-yl}-N-[(2S)-1,1,1-trifluoropropan-2-yl]acetamide;
2-[(4S)-5′-amino-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{4′-[(dimethylamino)methyl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-[4′-(morpholin-4-ylmethyl)-2,5-dioxo-2′,3′-dihydro-1H-spiro[imidazolidine-4,1′-inden]-1-yl]-N-[(2S)-1,1,1-trifluoropropan-2-yl]acetamide;
2-[2,5-dioxo-4′-(pyrrolidin-1-ylmethyl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-(4′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
methyl trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylate;
trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylic acid;
trans-4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]cyclohexanecarboxylic acid;
2-[(4R)-7′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
trans-4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}cyclohexanecarboxylic acid;
2-[(4S)-7′-bromo-2,5-dioxo-2′,3′-dihydro-H-spiro[imidazolidinne-4,1′-inden]-1-yl]-N-(4-fluorobenzyl)-N-[(2S)-1,1,1-trifluoropropan-2-yl]acetamide;
ethyl ({4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]piperidin-1-yl}sulfonyl)carbamate;
2-[(4S)-5′-{[(2-cyanoethyl)carbamoyl]amino}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{(4S)-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(4S)-5′-[(methylcarbamoyl)amino]-2,3′,5-trioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(3′S,4S)-3′-hydroxy-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(3′R,4S)-3′-fluoro-5′-[1-(2-hydroxyethyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
2-[4′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4R)-5′-bromo-6′-fluoro-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{(3′R,4S)-3′-fluoro-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{7′-[(methylcarbamoyl)amino]-2,2′,5-trioxo-2′,3′-dihydro-1H, 1′H-spiro[imidazolidine-4,4′-quinolin]-1-yl}-N-[(2S)-1,1,1-trifluoropropan-2-yl]acetamide;
tert-butyl (4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetate;
(4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetic acid;
{4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
(4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}cyclohexylidene)acetic acid;
N-(4-fluorobenzyl)-2-[(3′R,4S)-3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-[(4R)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(3′E,4S)-3′-(hydroxyimino)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-[(3′R,4S)-3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{4-[(3′R,4S)-3′,6′-difluoro-1-(2-{(4-fluorobenzyl)[(2S)-1,1,1-trifluoropropan-2-yl]amino}-2-oxoethyl)-2,5-dioxo-2′,3′-dihydro-H-spiro[imidazolidine-4,1′-inden]-5′-yl]-1H-pyrazol-1-yl}-N,N-dimethylacetamide;
2-[(3′S,4R)-3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{4-[(3′S,4R)-3′,6′-difluoro-1-(2-{(4-fluorobenzyl)[(2S)-1,1,1-trifluoropropan-2-yl]amino}-2-oxoethyl)-2,5-dioxo-2′,3′-dihydro-H-spiro[imidazolidine-4,1′-inden]-5′-yl]-1H-pyrazol-1-yl}-N,N-dimethylacetamide;
ethyl [(4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}piperidin-1-yl)sulfonyl]carbamate;
tert-butyl 4-[{[(4 S)-5′-bromo-2,5-dioxo-2′,3′-dihydro-1H-spiro[imidazolidine-4,1′-inden]-1-yl]acetyl}(4-fluorobenzyl)amino]piperidine-1-carboxylate;
ethyl ({4-[{[(4S)-5′-amino-2,5-dioxo-2′,3′-dihydro-1H-spiro[imidazolidine-4,1′-inden]-1-yl]acetyl}(4-fluorobenzyl)amino]piperidin-1-yl}sulfonyl)carbamate;
ethyl ({3,3-difluoro-4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]pyrrolidin-1-yl}sulfonyl)carbamate;
ethyl ({3,3-difluoro-4-[(4-fluorobenzyl) {[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]acetyl}amino]pyrrolidin-1-yl}sulfonyl)carbamate;
2-{(3′R,4S)-5′-[1-(difluoromethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-[2,5-dioxo-4′-(piperidin-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(4S)-7′-hydroxy-2,5-dioxo-2′,3′-dihydro-1H-spiro[imidazolidine-4,1′-inden]-1-yl]-N-[(2S)-1,1,1-trifluoropropan-2-yl]acetamide;
tert-butyl {4-[{[(4S)-5′-bromo-2,5-dioxo-2′,3′-dihydro-1H-spiro[imidazolidine-4,1′-inden]-1-yl]acetyl}(4-fluorobenzyl)amino]cyclohexylidene}acetate;
tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate;
{4-[{[(4S)-5′-amino-2,5-dioxo-2′,3′-dihydro-1H-spiro[imidazolidine-4,1′-inden]-1-yl]acetyl}(4-fluorobenzyl)amino]cyclohexylidene}acetic acid;
2-[(4S)-5′-bromo-6′-fluoro-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3′R,4R)-5′-bromo-6′-fluoro-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3′S,4S)-5′-bromo-6′-fluoro-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]-N-(tetrahydro-2H-pyran-4-yl)acetamide;
2-(2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-{6′-fluoro-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
2-{(3′R,4S)-3′,6′-difluoro-5′-[(methylcarbamoyl)amino]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
methyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate;
{4-[(4-fluorobenzyl) {[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
N-(4-fluorobenzyl)-2-[(4R)-7′-hydroxy-2,5-dioxo-2′,3′-dihydro-1H-spiro[imidazolidine-4,1′-inden]-1-yl]-N-[(2S)-1,1,1-trifluoropropan-2-yl]acetamide;
tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetate;
{4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetic acid;
2-[(4S)-2,5-dioxo-5′-(1H-pyrazol-5-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
4-[(4-fluorobenzyl) {[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexanecarboxylic acid;
2-[(4S)-5′-{1-[2-(dimethylamino)ethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
tert-butyl 4-({4-[(4S)-1-(2-{(4-fluorobenzyl)[(2S)-1,1,1-trifluoropropan-2-yl]amino}-2-oxoethyl)-2,5-dioxo-2′,3′-dihydrospiro[imidazolidine-4,1′-inden]-5′-yl]-1H-pyrazol-1-yl}methyl)piperidine-1-carboxylate;
2-{(4S)-2,5-dioxo-5′-[1-(piperidin-4-ylmethyl)-1H-pyrazol-4-yl]-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-{6-[(1-methyl-1H-pyrazol-4-yl)amino]-1,1-dioxido-2′,5′-dioxo-1′H-spiro[1-benzothiophene-3,4′-imidazolidin]-1′-yl}-N-[(2S)-1,1,1-trifluoropropan-2-yl]acetamide;
N-(4-fluorobenzyl)-2-(6-methoxy-1,1-dioxido-2′,5′-dioxo-1′H-spiro[1-benzothiophene-3,4′-imidazolidin]-1′-yl)-N-[(2S)-1,1,1-trifluoropropan-2-yl]acetamide;
N-(4-fluorobenzyl)-2-[(3 S)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide;
N-(4-fluorobenzyl)-2-[(3R)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide;
N-benzyl-N-(1-cyclopropylethyl)-2-(2,5-dioxo-2′,3′-dihydro-1H-spiro[imidazolidine-4,1′-inden]-1-yl)acetamide;
N-(1-cyclopropylethyl)-2-(2,5-dioxo-2′,3′-dihydro-1H-spiro[imidazolidine-4,1′-inden]-1-yl)-N-(1H-imidazol-4-ylmethyl)acetamide;
N-benzyl-N-(1-cyclopropylethyl)-2-(2,2-dimethyl-2′,5′-dioxo-1′H-spiro[1-benzofuran-3,4′-imidazolidin]-1′-yl)acetamide;
N-benzyl-N-(1-cyclopropylethyl)-2-(1′,3-dimethyl-2,2′,5-trioxo-1′,2′-dihydro-1H-spiro[imidazolidine-4,3′-indol]-1-yl)acetamide;
N-benzyl-N-(1-cyclopropylethyl)-2-(2,5-dioxo-2′,3′-dihydro-1H-spiro[imidazolidine-4,1′-inden]-1-yl)-2-methylpropanamide;
N-benzyl-N-(1-cyclopropylethyl)-2-[2,5-dioxo-3-(prop-2-en-1-yl)-2′,3′-dihydro-1H-spiro[imidazolidine-4,1′-inden]-1-yl]acetamide;
N-(1-cyclopropylethyl)-2-(2,5-dioxo-2′,3′-dihydro-1H-spiro[imidazolidine-4,1′-inden]-1-yl)-N-(2,2,2-trifluoroethyl)acetamide;
N-(1-cyclopropylethyl)-2-(2,5-dioxo-2′,3′-dihydro-1H-spiro[imidazolidine-4,1′-inden]-1-yl)-N-[3-(trifluoromethoxy)benzyl]acetamide;
N-(but-2-yn-1-yl)-N-(1-cyclopropylethyl)-2-(2,5-dioxo-2′,3′-dihydro-1H-spiro[imidazolidine-4,1′-inden]-1-yl)acetamide;
N-(1-cyclopropylethyl)-N-{[6-(difluoromethoxy)naphthalen-2-yl]methyl}-2-(2,5-dioxo-2′,3′-dihydro-1H-spiro[imidazolidine-4,1′-inden]-1-yl)acetamide;
N-benzyl-N-(1-cyclopropylethyl)-2-[5′-(methylsulfonyl)-2,5-dioxo-2′,3′-dihydro-1H-spiro[imidazolidine-4,1′-inden]-1-yl]acetamide;
N-benzyl-N-(1-cyclobutylethyl)-2-(7′-methoxy-2,5-dioxo-2′,3′-dihydro-1H-spiro[imidazolidine-4,1′-inden]-1-yl)acetamide;
N-benzyl-N-(1-cyclopropylethyl)-2-(1-methyl-2′,5′-dioxo-5,6-dihydro-1H,1′H-spiro[cyclopenta[c]pyrazole-4,4′-imidazolidin]-1′-yl)acetamide;
N-benzyl-N-(1-cyclopropylethyl)-2-[5′-(hydroxymethyl)-3-methyl-2,5-dioxo-2′,3′-dihydro-1H-spiro[imidazolidine-4,1′-inden]-1-yl]acetamide;
N-[(1S)-1-cyclobutylethyl]-2-[(4R)-2,5-dioxo-2′,3′-dihydro-1H-spiro[imidazolidine-4,1′-inden]-1-yl]-N-(prop-2-yn-1-yl)acetamide;
N³-[(1S)-1-cyclobutylethyl]-N³-{[(4R)-2,5-dioxo-2′,3′-dihydro-1H-spiro[imidazolidine-4,1′-inden]-1-yl]acetyl}-beta-alaninamide;
N-(biphenyl-4-ylmethyl)-N-[(1S)-1-cyclobutylethyl]-2-[(4R)-2,5-dioxo-2′,3′-dihydro-1H-spiro[imidazolidine-4,1′-inden]-1-yl]acetamide;
N²-[(1S)-1-cyclobutylethyl]-N²-{[(4R)-2,5-dioxo-2′,3′-dihydro-1H-spiro[imidazolidine-4,1′-inden]-1-yl]acetyl}glycinamide;
N-[(1S)-1-cyclobutylethyl]-2-[(4R)-2,5-dioxo-2′,3′-dihydro-1H-spiro[imidazolidine-4,1′-inden]-1-yl]-N-[(1-methyl-2-oxo-1,2-dihydropyridin-4-yl)methyl]acetamide;
N-benzyl-N-[(1R)-1-cyclopropylethyl]-2-[(4R)-2,5-dioxo-2′,3′-dihydro-1H-spiro[imidazolidine-4,1′-inden]-1-yl]acetamide;
N-benzyl-N-[(1S)-1-cyclopropylethyl]-2-[(4S)-2,5-dioxo-2′,3′-dihydro-1H-spiro[imidazolidine-4,1′-inden]-1-yl]acetamide;
N-benzyl-N-[(1S)-1-cyclopropylethyl]-2-[(4R)-2,5-dioxo-2′,3′-dihydro-1H-spiro[imidazolidine-4,1′-inden]-1-yl]acetamide;
N-benzyl-N-[(1R)-1-cyclopropylethyl]-2-[(4S)-2,5-dioxo-2′,3′-dihydro-1H-spiro[imidazolidine-4,1′-inden]-1-yl]acetamide;
N-[4-(acetylamino)benzyl]-N-[(1S)-1-cyclobutylethyl]-2-[(4R)-2,5-dioxo-2′,3′-dihydro-1H-spiro[imidazolidine-4,1′-inden]-1-yl]acetamide;
N-[(1S)-1-cyclobutylethyl]-2-[(4R)-2,5-dioxo-2′,3′-dihydro-1H-spiro[imidazolidine-4,1′-inden]-1-yl]-N-(4-ethynylbenzyl)acetamide;
N-[(1S)-1-cyclobutylethyl]-2-[(4R)-2,5-dioxo-2′,3′-dihydro-1H-spiro[imidazolidine-4,1′-inden]-1-yl]-N-({1-[2-(methylamino)-2-oxoethyl]-1H-1,2,3-triazol-4-yl}methyl)acetamide;
2-{4-[([(1S)-1-cyclobutylethyl]{[(4R)-2,5-dioxo-2′,3′-dihydro-1H-spiro[imidazolidine-4,1′-inden]-1-yl]acetyl}amino)methyl]-1H-1,2,3-triazol-1-yl}-N,N-dimethylacetamide;
N³-[(1S)-1-cyclobutylethyl]-N³-{[(4R)-2,5-dioxo-2′,3′-dihydro-1H-spiro[imidazolidine-4,1′-inden]-1-yl]acetyl}-N-methyl-beta-alaninamide;
N-benzyl-N-(1-cyclopropylethyl)-2-[5′-(6-hydroxypyridin-3-yl)-2,5-dioxo-2′,3′-dihydro-1H-spiro[imidazolidine-4,1′-inden]-1-yl]acetamide;
N-benzyl-N-[(1S)-1-cyclopropylethyl]-2-[5′-(formylamino)-2,5-dioxo-2′,3′-dihydro-1H-spiro[imidazolidine-4,1′-inden]-1-yl]acetamide;
N-benzyl-2-[(4R)-5′-bromo-2,5-dioxo-2′,3′-dihydro-1H-spiro[imidazolidine-4,1′-inden]-1-yl]-N-[(1S)-1-cyclopropylethyl]acetamide;
2-[1,1-dioxido-2′,5′-dioxo-6-(pyridin-3-yl)-1H-spiro[1-benzothiophene-3,4′-imidazolidin]-1′-yl]-N-(4-fluorobenzyl)-N-[(2S)-1,1,1-trifluoropropan-2-yl]acetamide;
N-(4-fluorobenzyl)-2-[(3′S,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
(4-{[(5′-bromo-6′-fluoro-2,5-dioxo-2′,3′-dihydro-1H-spiro[imidazolidine-4,1′-inden]-1-yl)acetyl](4-fluorobenzyl)amino}cyclohexylidene)acetic acid;
{4-[(4-fluorobenzyl) {[6′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
tert-butyl (4-{[(5′-bromo-6′-fluoro-2,5-dioxo-2′,3′-dihydro-1H-spiro[imidazolidine-4,1′-inden]-1-yl)acetyl](4-fluorobenzyl)amino}cyclohexylidene)acetate; and pharmaceutically acceptable salts thereof.

In another aspect, the present invention relates to compounds of Formula (XIa) or (XIb),

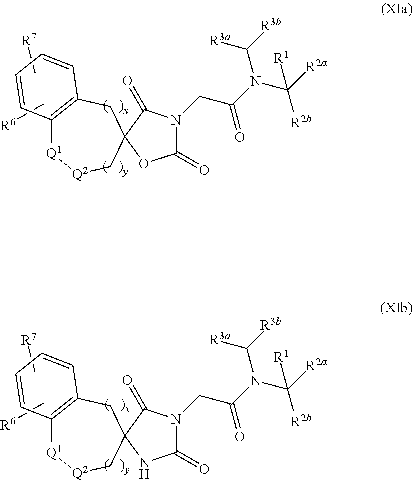

or a stereoisomer, tautomer or pharmaceutically acceptable salt thereof,
wherein
[symbol] is —C(R¹⁰)₂—C(R¹⁴)₂—, —O—C(R¹⁴)₂—, —O—C(O)—, —S(O)₂—C(R¹⁴)₂—, —S—C(R¹⁴)₂—, —NR⁹—C(O)—, —NR⁹—C(R¹⁴)₂—, —C(R¹⁰)₂—O—, —C(R¹⁰)₂—, or —C(R¹⁰)═C(R¹⁴)—;
R¹ is carbocyclyl or heterocyclyl;
R^{2a} and R^{2b} are each independently H, D, or C₁-C₆ alkyl;
R^{3a} is hydrogen, C(O)NH₂, C₁-C₆ alkyl, aryl, cycloalkyl or heterocyclyl; and
R^{3b} is C₁-C₆ alkyl, aryl, cycloalkyl or heterocyclyl; or
R^{3a} and R^{3b} taken together with the carbon to which they are attached form an arene, cycloalkane, or heterocycle;
R⁶ and R⁷ are each independently H, halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR²S(O)₂OR¹², —NHC(═O)NR²S(O)₂N(R¹²)₂, —NHC(═S)N(R¹²)₂, —NHC(═N—C≡N)NR¹², —NHC(═N—C≡N)SR¹², or —NHS(O)ₘR¹²;
R⁹ is independently H or C₁-C₆ alkyl;
R¹⁰, at each occurrence, is independently H, —OH, halo, —CN, —CO₂R¹², —C(═O)NHR¹³, —NHR¹², C₁-C₆ alkyl or alkoxy; or two R¹⁰ taken together form oxo or ═N—OR¹¹;
R¹¹ and R¹³ are each independently H, —OH, or C₁-C₆ alkyl;
R¹², at each occurrence, is independently H, C₁-C₆ alkyl, aryl, cycloalkyl, or heterocyclyl;
R¹⁴, at each occurrence, is independently H or C₁-C₆ alkyl;
m, at each occurrence, is independently 0, 1 or 2; and
x, and y are each independently 0 or 1, wherein x and y are selected such that the sum of x+y is 0 or 1;

with the proviso that R^{3a} and R^{3b} are not cyclopropyl and methyl, respectively, when R¹ is unsubstituted phenyl, R⁶ and R¹ are H; x is 0 or 1, y is 0, and [symbol] is —C(R¹⁰)₂—C(R¹⁴)₂—; and

with the proviso that R^{3a} and R^{3b} taken together with the carbon to which they are attached do not form tetrahydrothiophene 1,1-dioxide or tetrahydrothiophene when R¹ is unsubstituted phenyl or R⁶ and R are H.

In one embodiment of Formula (XIa) or (XIb), [symbol] is —C(R¹⁰)₂—C(R¹⁴)₂—, —O—C(R¹⁴)₂—, —O—C(O)—, —S(O)₂—C(R¹⁴)₂—, —S—C(R¹⁴)₂—, —NR⁹—C(O)—, —NR⁹—C(R¹⁴)₂—, —C(R¹⁰)₂—O—, —C(R¹⁰)₂—, or —C(R¹⁰)═C(R¹⁴)—. In another embodiment of Formula (XIa) or (XIb), [symbol] is C(R¹⁰)₂—C(R¹⁴)₂. In another embodiment of Formula (XIa) or (XIb), [symbol] is —O—C(R¹⁴)₂—. In another embodiment of Formula (XIa) or (XIb), [symbol] is —O—C(O)—. In another embodiment of Formula (XIa) or (XIb), [symbol] is —S(O)₂—C(R¹⁴)₂—. In another embodiment of Formula (XIa) or (XIb), [symbol] is —S—C(R¹⁴)₂—. In another embodiment of Formula (XIa) or (XIb), [symbol] is —NR⁹—C(O)—. In another embodiment of Formula (XIa) or (XIb), [symbol] is —NR⁹—C(R¹⁴)₂—. In another embodiment of Formula (XIa) or (XIb), [symbol] is —C(R¹⁰)₂—O—. In another embodiment of Formula (XIa) or (XIb), [symbol] is —C(R¹⁰)₂—. In another embodiment of Formula (XIa) or (XIb), [symbol] is —C(R¹⁰)═C(R¹⁴)—.

In one embodiment of Formula (XIa) or (XIb), R¹ is carbocyclyl or heterocyclyl. In another embodiment of Formula (XIa) or (XIb), R¹ is carbocyclyl. In another embodiment of Formula (XIa) or (XIb), R¹ is heterocyclyl. In another embodiment of Formula (XIa) or (XIb), R¹ is phenyl, naphthyl, cyclopropyl, or cyclobutyl. In another embodiment of Formula (XIa) or (XIb), R¹ is phenyl. In another embodiment of Formula (XIa) or (XIb), R¹ is phenyl, which is unsubstituted. In another embodiment of Formula (XIa) or (XIb), R¹ is phenyl, which is substituted. In another embodiment of Formula (XIa) or (XIb), R¹ is phenyl, which is substituted with F, Cl, Br, CN, —NR_{g}Rₕ, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, aryl, and/or haloalkyl; wherein alkyl, alone or part of a group, is optionally substituted with F, C(═O)NR_{g}Rₕ, or CN. In another embodiment of Formula (XIa) or (XIb), R¹ is phenyl, which is substituted with F, Cl, Br, CN, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, and/or haloalkyl; wherein alkyl, alone or part of a group, is optionally substituted with F. In another embodiment of Formula (XIa) or (XIb), R¹ is phenyl, which is substituted with F. In another embodiment of Formula (XIa) or (XIb), R¹ is tetrahydropyranyl, thiazolyl, oxazolyl, thiadiazolyl, pyrimidinyl, thiophenyl, furanyl, triazolyl, indolyl, imidazolyl, or pyridinyl. In another embodiment of Formula (XIa) or (XIb), R¹ is tetrahydropyranyl, thiazolyl, oxazolyl, thiadiazolyl, pyrimidinyl, thiophenyl, furanyl, triazolyl, indolyl, imidazolyl, or pyridinyl; which is unsubstituted. In another embodiment of Formula (XIa) or (XIb), R¹ is tetrahydropyranyl, thiazolyl, oxazolyl, thiadiazolyl, pyrimidinyl, thiophenyl, furanyl, triazolyl, indolyl, imidazolyl, or pyridinyl; which is substituted. In another embodiment of Formula (XIa) or (XIb), R¹ is tetrahydropyranyl, thiazolyl, oxazolyl, thiadiazolyl, pyrimidinyl, thiophenyl, furanyl, triazolyl, indolyl, imidazolyl, or pyridinyl; which is substituted with F, Cl, Br, CN, —NR_{g}Rₕ, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, aryl, and/or haloalkyl; wherein alkyl, alone or part of a group, is optionally substituted with F, C(═O)NR_{g}Rₕ, or CN.

In one embodiment of Formula (XIa) or (XIb), R^{2a} and R^{2b} are each independently H, D, or C₁-C₆ alkyl. In another embodiment of Formula (XIa) or (XIb), R^{2a} and R^{2b} are each independently H. In another embodiment of Formula (XIa) or (XIb), R^{2a} is H and R^{2b} is C₁-C₆ alkyl. In another embodiment of Formula (XIa) or (XIb), R^{2a} is H and R^{2b} is C₁-alkyl.

In one embodiment of Formula (XIa) or (XIb), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; or R^{3a} and R^{3b} taken together with the carbon to which they are attached form arene, cycloalkane, or heterocycle. In another embodiment of Formula (XIa) or (XIb), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; wherein C₁-C₆ alkyl, is optionally substituted with F, —CN, —NR_{g}Rₕ, —OR_{g}, or —SO₂R_{g}; wherein R_{g} and Rₕ are the same or different and independently hydrogen or alkyl; wherein aryl, cycloalkyl and heterocyclyl are optionally substituted with alkyl, —SO₂NR_{g}C(═O)ORₕ, —SO₂NR_{g}Rₕ, —C(═O)R_{g}, —C(═O)OR_{g}, or —C(═O)NR_{g}Rₕ; wherein R_{g} and Rₕ are the same or different and independently hydrogen or alkyl; wherein alkyl, alone or part of a group, is optionally substituted with aryl or —C(═O)OH.

In another embodiment of Formula (XIa) or (XIb), R^{3a} is C₁-C₆ alkyl; and R^{3b} is C₁-C₆ alkyl; wherein the C₁-C₆ alkyl is optionally substituted with one or more F, —CN, alkoxy, or —SO₂R_{g}. In another embodiment of Formula (XIa) or (XIb), R^{3a} is C₁-C₆ alkyl; and R^{3b} is CF₃. In another embodiment of Formula (XIa) or (XIb), R^{3a} is CH₃; and R^{3b} is CF₃. In another embodiment of Formula (XIa) or (XIb), R^{3a} is hydrogen; and R^{3b} is C₁-C₆ alkyl; wherein the C₁-C₆ alkyl is optionally substituted with one or more F, —CN, alkoxy, or SO₂R_{g}. In another embodiment of Formula (XIa) or (XIb), R^{3a} is hydrogen; and R^{3b} is cycloalkyl. In another embodiment of Formula (XIa) or (XIb), R^{3a} is hydrogen; and R^{3b} is cyclopropyl. In another embodiment of Formula (XIa) or (XIb), R^{3a} is C₁-C₆ alkyl; wherein the C₁-C₆ alkyl is CH₃; and R^{3b} is cycloalkyl. In another embodiment of Formula (XIa) or (XIb), R^{3a} is CH₃; and R^{3b} is cyclopropyl. In another embodiment of Formula (XIa) or (XIb), R^{3a} is CF₃; and R^{3b} is cyclopropyl. In another embodiment of Formula (XIa) or (XIb), R^{3a} is cycloalkyl; and R^{3b} is cycloalkyl. In another embodiment of Formula (XIa) or (XIb), R^{3a} is cyclopropyl; and R^{3b} is cyclopropyl. In another embodiment of Formula (XIa) or (XIb), R^{3a} is C₁-C₆ alkyl; and R^{3b} is heterocyclyl. In another embodiment of Formula (XIa) or (XIb), R^{3a} is hydrogen; and R^{3b} is aryl. In another embodiment of Formula (XIa) or (XIb), R^{3a} and R^{3b} taken together with the carbon to which they are attached form arene, cycloalkane, or heterocycle. In another embodiment of Formula (XIa) or (XIb), R^{3a} and R^{3b} taken together with the carbon to which they are attached form arene. In another embodiment of Formula (XIa) or (XIb), R^{3a} and R^{3b} taken together with the carbon to which they are attached form cycloalkane. In another embodiment of Formula (XIa) or (XIb), R^{3a} and R^{3b} taken together with the carbon to which they are attached form heterocycle. In another embodiment of Formula (XIa) or (XIb), R^{3a} and R^{3b} taken together with the carbon to which they are attached form cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, or benzene. In another embodiment of Formula (XIa) or (XIb), R^{3a} and R^{3b} taken together with the carbon to which they are attached form cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, or benzene wherein cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, and benzene are optionally substituted with alkyl, haloalkyl, —F, —CN, —NR_{g}Rₕ, —NR_{g}C(═O)Rₕ, —NR_{g}SO₂Rₕ, —OR_{g}, —SO₂R_{g}, —NR_{g}SO₂NR_{g}C(═O)Rₕ, —SO₂NR_{g}C(═O)ORₕ, —SO₂NR_{g}Rₕ, —C(═O)R_{g}, —C(═O)OR_{g}, or —C(═O)NR_{g}Rₕ; wherein R_{g} and Rₕ are the same or different and independently hydrogen, alkyl, or haloalkyl; wherein each alkyl, alone or part of a group, is optionally substituted with CN, alkyl, —SO₂NH₂, C(═O)NHCH₃, —C(═O)OC(CH₃)₃, —C(═O)OCH₃, or —C(═O)OH.

In one embodiment of Formula (XIa) or (XIb), R⁶ and R⁷ are each independently H, halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═S)N(R¹²)₂, —NHC(═N—C≡N)NR¹², —NHC(═N—C≡N)SR¹², or —NHS(O)ₘR¹². In another embodiment of Formula (XIa) or (XIb), R⁷ is H and R⁶ is H, halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═S)N(R¹²)₂, —NHC(═N—C≡N)NR¹², —NHC(═N—C≡N)SR¹², or —NHS(O)ₘR¹². In another embodiment of Formula (XIa) or (XIb), R⁷ is H and R⁶ is —N(R¹²)₂, —C(═O)NHR¹², —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═O)N(R¹²)₂, —NHC(═S)N(R¹²)₂, —NHC(═N—C≡N)NR¹², —NHC(═N—C≡N)SR¹², or —NHS(O)ₘR¹². In another embodiment of Formula (XIa) or (XIb), R⁷ is H and R⁶ is heterocyclyl. In another embodiment of Formula (XIa) or (XIb), R⁷ is H and R⁶ is pyrazolyl. In another embodiment of Formula (XIa) or (XIb), R⁷ is H and R⁶ is —NHC(═O)NHR¹².

In one embodiment of Formula (XIa) or (XIb), R⁹ is independently H or C₁-C₆ alkyl. In another embodiment of Formula (XIa) or (XIb), R⁹ is independently H. In another embodiment of Formula (XIa) or (XIb), R⁹ is independently C₁-C₆ alkyl.

In one embodiment of Formula (XIa) or (XIb), R¹⁰, at each occurrence, is independently H, —OH, halo, —CN, —CO₂R¹², —C(═O)NHR¹³, —NHR¹², C₁-C₆ alkyl or alkoxy; or two R¹⁰ taken together form oxo or ═N—OR¹¹. In another embodiment of Formula (XIa) or (XIb), R¹⁰, at each occurrence, is independently H, —OH, halo, —CN, —CO₂R¹², —C(═O)NHR¹³, —NHR¹², C₁-C₆ alkyl or alkoxy; or two R¹⁰ taken together form oxo or ═N—OR¹¹. In another embodiment of Formula (XIa) or (XIb), R¹⁰, at each occurrence, is independently H. In another embodiment of Formula (XIa) or (XIb), R¹⁰, at each occurrence, is independently H or —OH. In another embodiment of Formula (XIa) or (XIb), R¹⁰, at each occurrence, is independently H or halo. In another embodiment of Formula (XIa) or (XIb), R¹⁰, at each occurrence, is independently H or F. In another embodiment of Formula (XIa) or (XIb), one R¹⁰ is independently H, and the remaining is F. In another embodiment of Formula (XIa) or (XIb), R¹⁰, at each occurrence, is independently H or —CN. In another embodiment of Formula (XIa) or (XIb), R¹⁰, at each occurrence, is independently H or —CO₂R¹². In another embodiment of Formula (XIa) or (XIb), R¹⁰, at each occurrence, is independently H or —C(═O)NHR¹³. In another embodiment of Formula (XIa) or (XIb), R¹⁰, at each occurrence, is independently H or —NHR¹². In another embodiment of Formula (XIa) or (XIb), R¹⁰, at each occurrence, is independently H or C₁-C₆ alkyl. In another embodiment of Formula (XIa) or (XIb), R¹⁰, at each occurrence, is independently H or alkoxy. In another embodiment of Formula (XIa) or (XIb), two R¹⁰ taken together form oxo. In another embodiment of Formula (XIa) or (XIb), two R¹⁰ taken together form ═N—OR¹¹.

In one embodiment of Formula (XIa) or (XIb), R¹¹ and R¹³ are each independently H, —OH, or C₁-C₆ alkyl. In another embodiment of Formula (XIa) or (XIb), R¹¹ and R¹³ are each independently H. In another embodiment of Formula (XIa) or (XIb), R¹¹ and R¹³ are each independently —OH. In another embodiment of Formula (XIa) or (XIb), R¹¹ and R¹³ are each independently C₁-C₆ alkyl.

In one embodiment of Formula (XIa) or (XIb), R¹², at each occurrence, is independently H, C₁-C₆ alkyl, aryl, cycloalkyl, or heterocyclyl. In another embodiment of Formula (XIa) or (XIb), R¹², at each occurrence, is independently H, C₁-C₆ alkyl, aryl, cycloalkyl, or heterocyclyl; wherein C₁-C₆ alkyl, aryl, cycloalkyl, and heterocyclyl are optionally substituted with F, alkyl, alkoxy, cycloalkyl, haloalkyl, heterocyclyl, heterocyclylalkyl, oxo, CN, —NR_{g}Rₕ, —NR_{g}C(═O)Rₕ, —NR_{g}C(═O)ORₕ, —OR_{g}, —SO₂R_{g}, —SO₂NR_{g}Rₕ, —C(═O)R_{g}, —C(═O)OR_{g}, or —C(═O)NR_{g}Rₕ; wherein R_{g} and Rₕ are the same or different and independently hydrogen, alkyl, alkoxy, aryl, cycloalkyl, and/or haloalkyl.

In one embodiment of Formula (XIa) or (XIb), R¹⁴, at each occurrence, is independently H or C₁-C₆ alkyl. In another embodiment of Formula (XIa) or (XIb), R¹⁴, at each occurrence, is independently H. In another embodiment of Formula (XIa) or (XIb), R¹⁴, at each occurrence, is independently C₁-C₆ alkyl.

In one embodiment of Formula (XIa) or (XIb), m, at each occurrence, is independently 0, 1 or 2. In another embodiment of Formula (XIa) or (XIb), m, at each occurrence, is independently 0. In another embodiment of Formula (XIa) or (XIb), m, at each occurrence, is independently 1. In another embodiment of Formula (XIa) or (XIb), m, at each occurrence, is independently 2.

In one embodiment of Formula (XIa) or (XIb), x, and y are each independently 0 or 1, wherein x and y are selected such that the sum of x+y is 0 or 1. In another embodiment of Formula (XIa) or (XIb), x is 0 and y is 0. In another embodiment of Formula (XIa) or (XIb), x is 0 and y is 1. In another embodiment of Formula (XIa) or (XIb), x is 1 and y is 0.

Still another embodiment pertains to compounds of Formula (XIa), selected from the group consisting of:
N-benzyl-N-(1-cyclopropylethyl)-2-(2′,4′-dioxo-2,3-dihydrospiro[indene-1,5′-ozazolidine]-3′-yl)acetamide;
N-benzyl-2-(5-bromo-2′,4′-dioxo-2,3-dihydrospiro[indene-1,5′-ozazolidine]-3′-yl)-N-(1-cyclopropylethyl)acetamide;
N-benzyl-2-(2′,4′-dioxo-2,3-dihydrospiro[indene-1,5′-ozazolidine]-3′-yl)-N-(pentan-3-yl)acetamide;
N-benzyl-N-(1-cyclobutylethyl)-2-(2′,4′-dioxo-2,3-dihydrospiro[indene-1,5′-ozazolidine]-3′-yl)acetamide;
N-(1-cyclopropylethyl)-2-(2′,4′-dioxo-2,3-dihydrospiro[indene-1,5′-ozazolidine]-3′-yl)-N-(4-fluorobenzyl)acetamide;
N-benzyl-N-(1-cyclopropylethyl)-2-(4-methoxy-2′,4′-dioxo-2,3-dihydrospiro[indene-1,5′-ozazolidine]-3′-yl)acetamide;
2-(5-bromo-2′,4′-dioxo-2,3-dihydrospiro[indene-1,5′-ozazolidine]-3′-yl)-N-(1-cyclopropylethyl)-N-(4-fluorobenzyl)acetamide;
N-benzyl-2-(5-cyano-2′,4′-dioxo-2,3-dihydrospiro[indene-1,5′-ozazolidine]-3′-yl)-N-(1-cyclopropylethyl)acetamide;
(S)-2-(5-acetamido-2′,4′-dioxo-2,3-dihydrospiro[indene-1,5′-ozazolidine]-3′-yl)-N-benzyl-N-(1-cyclopropylethyl)acetamide;
3′-(2-(benzyl(1-cyclopropylethyl)amino)-2-oxoethyl)-2′,4′-dioxo-2,3-dihydrospiro[indene-1,5′-ozazolidine]-5-carboxamide;
N-benzyl-N-(1-cyclopropylethyl)-2-(5-methoxy-2′,4′-dioxo-2,3-dihydrospiro[indene-1,5′-ozazolidine]-3′-yl)acetamide;
N-benzyl-N—((S)-1-cyclopropylethyl)-2-(5-(3-hydroxyprop-1-ynyl)-2′,4′-dioxo-2,3-dihydrospiro[indene-1,5′-ozazolidine]-3′-yl)acetamide;
N-benzyl-N—((S)-1-cyclopropylethyl)-2-(5-(methylsulfonamido)-2′,4′-dioxo-2,3-dihydrospiro[indene-1,5′-ozazolidine]-3′-yl)acetamide;
(S)-3′-(2-(benzyl(1-cyclopropylethyl)amino)-2-oxoethyl)-2′,4′-dioxo-2,3-dihydrospiro[indene-1,5′-ozazolidine]-5-carboxamide;
N-benzyl-N—((S)-1-cyclopropylethyl)-2-(5-(morpholinomethyl)-2′,4′-dioxo-2,3-dihydrospiro[indene-1,5′-ozazolidine]-3′-yl)acetamide;
N-benzyl-N—((S)-1-cyclopropylethyl)-2-(5-(3-methylureido)-2′,4′-dioxo-2,3-dihydrospiro[indene-1,5′-ozazolidine]-3′-yl)acetamide;
(S)—N-(3′-(2-(benzyl(1-cyclopropylethyl)amino)-2-oxoethyl)-2′,4′-dioxo-2,3-dihydrospiro[indene-1,5′-ozazolidine]-5-yl)-3-oxobutanamide;
N-benzyl-N—((S)-1-cyclopropylethyl)-2-(5-morpholino-2′,4′-dioxo-2,3-dihydrospiro[indene-1,5′-ozazolidine]-3′-yl)acetamide;
N-benzyl-2-(5-(2-cyanoacetamido)-2′,4′-dioxo-2,3-dihydrospiro[indene-1,5′-ozazolidine]-3′-yl)-N—((S)-1-cyclopropylethyl)acetamide;
N-benzyl-N—((S)-1-cyclopropylethyl)-2-(2′,4′-dioxo-5-(2-oxa-6-azaspiro[3.3]heptan-6-yl)-2,3-dihydrospiro[indene-1,5′-ozazolidine]-3′-yl)acetamide;
N-benzyl-N—((S)-1-cyclopropylethyl)-2-(2′,4′-dioxo-5-(piperidin-1-ylmethyl)-2,3-dihydrospiro[indene-1,5′-ozazolidine]-3′-yl)acetamide;
N-benzyl-N—((S)-1-cyclopropylethyl)-2-(2′,4′-dioxo-5-(piperazin-1-ylmethyl)-2,3-dihydrospiro[indene-1,5′-ozazolidine]-3′-yl)acetamide;
N-benzyl-N—((S)-1-cyclopropylethyl)-2-(5-((4-hydroxypiperidin-1-yl)methyl)-2′,4′-dioxo-2,3-dihydrospiro[indene-1,5′-ozazolidine]-3′-yl)acetamide;
(S)-2-amino-N-(3′-(2-(benzyl(1-cyclopropylethyl)amino)-2-oxoethyl)-2′,4′-dioxo-2,3-dihydrospiro[indene-1,5′-ozazolidine]-5-yl)-2-methylpropanamide;
(S)—N-benzyl-N-(1-cyclopropylethyl)-2-(2′,4′-dioxo-5-(piperazin-1-yl)-2,3-dihydrospiro[indene-1,5′-ozazolidine]-3′-yl)acetamide;
N-benzyl-N—((S)-1-cyclopropylethyl)-2-(5-hydroxy-2′,4′-dioxo-2,3-dihydrospiro[indene-1,5′-ozazolidine]-3′-yl)acetamide;
methyl 2-(3′-(2-(benzyl((S)-1-cyclopropylethyl)amino)-2-oxoethyl)-2′,4′-dioxo-2,3-dihydrospiro[indene-1,5′-ozazolidine]-5-yloxy)acetate;
N-benzyl-N—((S)-1-cyclopropylethyl)-2-(2′,4′-dioxo-5-(prop-2-ynyloxy)-2,3-dihydrospiro[indene-1,5′-ozazolidine]-3′-yl)acetamide;
(S)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)-3-methylbutanamide;
(2S)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-ylpropanamide;
2-(3′-(2-(benzyl((S)-1-cyclopropylethyl)amino)-2-oxoethyl)-2′,4′-dioxo-2,3-dihydrospiro[indene-1,5′-ozazolidine]-5-ylamino)acetic acid;
N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-cyano-3-oxobutanamide;
2-(3′-(2-(benzyl((S)-1-cyclopropylethyl)amino)-2-oxoethyl)-2′,4′-dioxo-2,3-dihydrospiro[indene-1,5′-ozazolidine]-5-yloxy)acetic acid;
N-benzyl-N—((S)-1-cyclopropylethyl)-2-(5-(oxetan-3-ylamino)-2′,4′-dioxo-2,3-dihydrospiro[indene-1,5′-ozazolidine]-3′-yl)acetamide;
N-benzyl-N—((S)-1-cyclopropylethyl)-2-(2′,4′-dioxo-5-(tetrahydro-2H-pyran-4-ylamino)-2,3-dihydrospiro[indene-1,5′-ozazolidine]-3′-yl)acetamide;
N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-hydroxypropanamide;
1-(3′-(2-(benzyl((S)-1-cyclopropylethyl)amino)-2-oxoethyl)-2′,4′-dioxo-2,3-dihydrospiro[indene-1,5′-ozazolidine]-5-yl)azetidine-3-carboxamide;
2-amino-N-(3′-(2-(benzyl(dicyclopropylmethyl)amino)-2-oxoethyl)-2′,4′-dioxo-2,3-dihydrospiro[indene-1,5′-ozazolidine]-5-yl)-2′-methylpropanamide;
N-benzyl-N-(dicyclopropylmethyl)-2-(5-(3-methylureido)-2′,4′-dioxo-2,3-dihydrospiro[indene-1,5′-ozazolidine]-3′-yl)acetamide;
N-benzyl-N—((S)-1-cyclopropylethyl)-2-(5-(2-(dimethylamino)ethoxy)-2′,4′-dioxo-2,3-dihydrospiro[indene-1,5′-ozazolidine]-3′-yl)acetamide;
2-(5-((1H-1,2,3-triazol-4-yl)methoxy)-2′,4′-dioxo-2,3-dihydrospiro[indene-1,5′-ozazolidine]-3′-yl)-N-benzyl-N—((S)-1-cyclopropylethyl)acetamide;
N-benzyl-N—((S)-1-cyclopropylethyl)-2-(2′,4′-dioxo-5-(6-oxo-1,6-dihydropyridin-3-yl)-2,3-dihydrospiro[indene-1,5′-ozazolidine]-3′-yl)acetamide;
N-benzyl-N—((S)-1-cyclopropylethyl)-2-(5-(2-hydroxyethoxy)-2′,4′-dioxo-2,3-dihydrospiro[indene-1,5′-ozazolidine]-3′-yl)acetamide;
3′-(2-(benzyl((S)-1-cyclopropylethyl)amino)-2-oxoethyl)-2′,4′-dioxo-2,3-dihydrospiro[indene-1,5′-ozazolidine]-5-ylboronic acid;
N-benzyl-N—((S)-1-cyclopropylethyl)-2-(5-(3-(hydroxymethyl)azetidin-1-yl)-2′,4′-dioxo-2,3-dihydrospiro[indene-1,5′-ozazolidine]-3′-yl)acetamide;
N—((S)-1-cyclopropylethyl)-2-(2′,4′-dioxo-5-(6-oxo-1,6-dihydropyridin-3-yl)-2,3-dihydrospiro[indene-1,5′-ozazolidine]-3′-yl)-N-(4-fluorobenzyl)acetamide;
1-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)cyclobutanecarboxamide;
2-(5-(2-cyanoacetamido)-2′,4′-dioxo-2,3-dihydrospiro[indene-1,5′-ozazolidine]-3′-yl)-N—((S)-1-cyclopropylethyl)-N-(4-fluorobenzyl)acetamide;
N—((S)-1-cyclopropylethyl)-N-(4-fluorobenzyl)-2-(5-(3-methylureido)-2′,4′-dioxo-2,3-dihydrospiro[indene-1,5′-ozazolidine]-3′-yl)acetamide;
3′-((4S)-3-benzyl-4-cyclopropyl-2-oxopentyl)-5-(1H-pyrazol-5-yl)-2,3-dihydrospiro[indene-1,5′-ozazolidine]-2′,4′-dione;
2-amino-N-(3′-(2-(benzyl((S)-1-cyclopropylethyl)amino)-2-oxoethyl)-4′-oxo-2,3-dihydrospiro[indene-1,5′-ozazolidine]-5-yl)-3-hydroxypropanamide;
(S)—N-(3′-(2-(benzyl(1-cyclopropylethyl)amino)-2-oxoethyl)-2′,4′-dioxo-2,3-dihydrospiro[indene-1,5′-ozazolidine]-5-yl)-2-hydroxy-2-methylpropan amide;
1-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)cyclopropanecarboxamide;
2-amino-N-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)-2-methylpropanamide;
(S)-2-(5-(azetidin-3-ylamino)-2′,4′-dioxo-2,3-dihydrospiro[indene-1,5′-ozazolidine]-3′-yl)-N-benzyl-N-(1-cyclopropylethyl)acetamide;
(2R)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-ylpropanamide;
(S)-2-(3′-(2-(benzyl(1-cyclopropylethyl)amino)-2-oxoethyl)-2′,4′-dioxo-2,3-dihydrospiro[indene-1,5′-ozazolidine]-5-ylcarboxamido)acetic acid;
2-((S)-5-acetamido-2′,4′-dioxo-2,3-dihydrospiro[indene-1,5′-ozazolidine]-3′-yl)-N-benzyl-N—((S)-1-cyclopropylethyl)acetamide;
2-((R)-5-acetamido-2′,4′-dioxo-2,3-dihydrospiro[indene-1,5′-ozazolidine]-3′-yl)-N-benzyl-N—((S)-1-cyclopropylethyl)acetamide;
N-benzyl-2-{5-[(methylcarbamoyl)amino]-3′,5′-dioxo-2,3-dihydrospiro[indene-1,2′-[1,4 ]ozazolidine]-4′-yl}-N-(pentan-3-yl)acetamide;
2-amino-N-(4-{[benzyl(pentan-3-ylcarbamoyl]methyl}-3′,5′-dioxo-2,3-dihydrospiro[indene-1,2′-[1,4]ozazolidine]-5-yl)-2-methylpropanamide;
2-{5-[(azetidin-3-ylmethyl)amino]-3′,5′-dioxo-2,3-dihydrospiro[indene-1,2′-[1,4]ozazolidine]-4′-yl}-N-benzyl-N-[(1S)-1-cyclopropylethyl]acetamide;
N-benzyl-N-[(1S)-1-cyclopropylethyl]-2-{5-[(1,3-dihydroxypropan-2-yl)oxy]-3′,5′-dioxo-2,3-dihydrospiro[indene-1,2′-[1,4]ozazolidine]-4′-yl}acetamide;
1-amino-N-[4′-({[(18)-1-cyclopropylethyl][(4-fluorophenyl)methyl]carbamoyl}methyl)-3′,5′-dioxo-2,3-dihydrospiro[indene-[1,2′-[1,4]ozazolidine]-5-yl]cyclobutane-1-carboxamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]azetidine-3-carboxamide;
4-{[4′-({benzyl[(1S)-1-cyclopropylethyl]carbamoyl}methyl)-3′,5′-dioxo-2,3-dihydrospiro[indene-1,2′-[1,4]ozazolidine]-5-ylmethyl]amino}benzoic acid;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-2,3-dihydroxypropanamide;
N-benzyl-2-{5-[(methylcarbamoyl)amino]-3′,5′-dioxo-2,3-dihydrospiro[indene-1,2′-[1,4]ozazolidine]-4′-yl}-N-(oxolan-3-yl)acetamide;
N-benzyl-N-[(1S)-1-cyclopropylethyl]-2-{5-[(1,1-dioxo-1λ⁶-thian-4-yl)amino]-3′,5′-dioxo-2,3-dihydrospiro[indene-1,2′-[1,4]ozazolidine]-4′-yl}acetamide;
2-{5-[(2R)-2-amino-2-cyclopropylacetamido]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-(cyclopropylmethyl)-2-{5-[(methylcarbamoyl)amino]-3′,5′-dioxo-2,3-dihydrospiro[indene-1,2′-[1,4]ozazolidine]-4′-yl}-N-[(2-methylphenyl)methyl]acetamide;
N-ethyl-2-{5-[(methylcarbamoyl)amino]-3′,5′-dioxo-2,3-dihydrospiro[indene-1,2′-[1,4]ozazolidine]-4′-yl}-N-[(2-methylphenyl)methyl]acetamide;
N-[(1S)-1-cyclopropylethyl]-2-{3′,5′-dioxo-5-[(pyrrolidin-3-yl)amino]-2,3-dihydrospiro[indene-1,2′-[1,4]ozazolidine]-4′-yl}-N-[(4-fluorophenyl)methyl]acetamide;
2-(5-{[(1-aminocyclopropyl)methyl]amino}-3′,5′-dioxo-2,3-dihydrospiro[indene-1,2′-[1,4]ozazolidine]-4′-yl)-N-[(1S)-1-cyclopropylethyl]-N-[(4-fluorophenyl)methyl]acetamide;
N-benzyl-2-{5-[(methylcarbamoyl)amino]-3′,5′-dioxo-2,3-dihydrospiro[indene-1,2′-[1,4]ozazolidine]-4′-yl}-N-(oxan-4-yl)acetamide;
N-benzyl-N-[(1R)-1-cyclopropyl-2,2,2-trifluoroethyl]-2-{5-[(methylcarbamoyl)amino]-3′,5′-dioxo-2,3-dihydrospiro[indene-1,2′-[1,4]ozazolidine]-4′-yl}acetamide;
N-benzyl-2-{5-[(methylcarbamoyl)amino]-3′,5′-dioxo-2,3-dihydrospiro[indene-1,2′-[1,4]ozazolidine]-4′-yl}-N-(oxan-3-yl)acetamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]ethanediamide;
2-{5-[(azetidin-3-yl)amino]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-benzyl-N-[(1S)-1-cyclopropylethyl]-2-{3′,5′-dioxo-5-[(2-oxopyrrolidin-3-yl)amino]-2,3-dihydrospiro[indene-[1,2′-[1,4]ozazolidine]-4′-yl}acetamide;
2-{[4′-({benzyl[(1S)-1-cyclopropylethyl]carbamoyl}methyl)-3′,5′-dioxo-2,3-dihydrospiro[indene-1,2′-[1,4]ozazolidine]-5-yl]carbamoyl}-2,2-dimethylacetic acid;
N-benzyl-N-[(1S)-1-cyclopropylethyl]-2-{5-[(2-hydroxyethyl)amino]-3′,5′-dioxo-2,3-dihydrospiro[indene-1,2′-[1,4]ozazolidine]-4′-yl}acetamide;
(2R)-2-amino-N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]propanamide;
{[4′-({benzyl[(1S)-1-cyclopropylethyl]carbamoyl}methyl)-3′,5′-dioxo-2,3-dihydrospiro[indene-1,2′-[1,4]ozazolidine]-5-yl]carbamoyl}formic acid;
(2S)-2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-hydroxypropanamide;
N-[(1S)-1-cyclopropylethyl]-2-{3′,5′-dioxo-5-[(piperidin-3-yl)amino]-2,3-dihydrospiro[indene-1,2′-[1,4]ozazolidine]-4′-yl}-N-[(4-fluorophenyl)methyl]acetamide;
N-benzyl-N-[(1S)-1-cyclopropylethyl]-2-{5-[(methylcarbamoyl)amino]-3′,5′-dioxo-2,3-dihydro(3,3-²H₂)spiro[indene-1,2′-[1,4]ozazolidine]-4′-yl}acetamide;
N-[(4-fluorophenyl)methyl]-2-{5-[(methylcarbamoyl)amino]-3′,5′-dioxo-2,3-dihydrospiro[indene-1,2′-[1,4]ozazolidine]-4′-yl}-N-[(2S)-1,1,1-trifluoropropan-2-yl]acetamide;
2-amino-N-[4′-({benzyl[(1R)-1-cyclopropyl-2,2,2-trifluoroethyl]carbamoyl}methyl)-3′,5′-dioxo-2,3-dihydrospiro[indene-1,2′-[1,4]ozazolidine]-5-yl]-2-methylpropanamide;
2-{5-[(methylcarbamoyl)amino]-3′,5′-dioxo-2,3-dihydrospiro[indene-1,2′-[1,4]ozazolidine]-4′-yl}-N-[(2-methylphenyl)methyl]-N-(2-methylpropyl)acetamide;
2-{5-[(4-aminooxolan-3-yl)amino]-3′,5′-dioxo-2,3-dihydrospiro[indene-1,2-[1,4]ozazolidine]-4′-yl}-N-[(1S)-1-cyclopropylethyl]-N-[(4-fluorophenyl)methyl]acetamide;
N-[(4-²H)benzyl]-N-[(1S)-1-cyclopropylethyl]-2-{5-[(methylcarbamoyl)amino]-3′,5′-dioxo-2,3-dihydrospiro[indene-1,2′-[1,4]ozazolidine]-4′-yl}acetamide;
N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-2-methyl-2-(methylamino)propanamide;
(2R)-2-amino-N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-benzyl-N-[(1S)-1-cyclopropylethyl]-2-[(1S)-5-[(methylcarbamoyl)amino]-3′,5′-dioxo-2,3-dihydrospiro[indene-1,2′-[1,4]ozazolidine]-4′-yl]acetamide;
N-benzyl-N-[(1S)-1-cyclopropylethyl]-2-[(1R)-5-[(methylcarbamoyl)amino]-3′,5′-dioxo-2,3-dihydrospiro[indene-1,2′-[1,4]ozazolidine]-4′-yl]acetamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-methylazetidine-3-carboxamide;
N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylazetidine-3-carboxamide;
2-{5-[(2R)-2-amino-2-cyclopropylacetamido]-3′,5′-dioxo-2,3-dihydrospiro[indene-1,2′-[1,4]ozazolidine]-4′-yl}-N-[(4-fluorophenyl)methyl]-N-[(2S)-1,1,1-trifluoropropan-2-yl]acetamide;
N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylazetidine-3-carboxamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-2-(dimethylamino)-2-methylpropanamide;
(2R)-2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-hydroxypropanamide;
2-{5-bromo-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
2-{5-[(3-aminocyclohexyl)amino]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-[1-(azetidin-3-yl)ethyl]-N-benzyl-2-{5-[(methylcarbamoyl)amino]-3′,5′-dioxo-2,3-dihydrospiro[indene-1,2′-[1,4]ozazolidine]-4′-yl}acetamide;
2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-2-methylpropanamide;
N-[(4-fluorophenyl)methyl]-N-[(2S)-1-methoxypropan-2-yl]-2-{5-[(methylcarbamoyl)amino]-3′,5′-dioxo-2,3-dihydrospiro[indene-1,2′-[1,4]ozazolidine]-4′-yl}acetamide;
3-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]oxetane-3-carboxamide;
2-amino-N-[(1S)-4′-({[(4-bromophenyl)methyl][(1S)-1-cyclopropylethyl]carbamoyl}methyl)-3′,5′-dioxo-2,3-dihydrospiro[indene-1,2′-[1,4]ozazolidine]-5-yl]-2-methylpropanamide;
2-amino-N-[(1R)-4′-({[(4-bromophenyl)methyl][(1S)-1-cyclopropylethyl]carbamoyl}methyl)-3′,5′-dioxo-2,3-dihydrospiro[indene-1,2′-[1,4]ozazolidine]-5-yl]-2-methylpropanamide;
2-{5-[(2R)-2-amino-2-(oxetan-3-yl)acetamido]-3′,5′-dioxo-2,3-dihydrospiro[indene-1,2′-[1,4]ozazolidine]-4′-yl}-N-benzyl-N-[(1S)-1-cyclopropylethyl]acetamide;
1-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3-difluorocyclobutane-1-carboxamide;
2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropan amide;
(2R)-2-amino-N-[(1S)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1R)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]-N-[(2S)-1,1,1-trifluoropropan-2-yl]acetamide;
N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]-N-[(2S)-1,1,1-trifluoropropan-2-yl]acetamide;
N-[(1S)-1-cyclopropylethyl]-N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]acetamide;
N-[(1S)-1-cyclopropylethyl]-N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]acetamide;
(2S)-2-amino-N-[(1S)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1S)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2S)-2-amino-N-[(1R)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1R)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
N-benzyl-N-[(1R)-1-cyclopropyl-2,2,2-trifluoroethyl]-2-(1S)-5-[(methylcarbamoyl)amino]-3′,5′-dioxo-2,3-dihydrospiro[indene-1,2′-[1,4]ozazolidine-4′-yl]acetamide;
N-benzyl-N-[(1R)-1-cyclopropyl-2,2,2-trifluoroethyl]-2-[(1R)-5-(methylcarbamoyl)amino]-3′,5′-dioxo-2,3-dihydrospiro[indene-1,2′-[1,4]ozazolidine]-4′-yl)acetamide;
(2R)-2-amino-N-[(1S)-4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1R)-4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1S)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1R)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(1R)-1-cyclopropyl-2,2,2-trifluoroethyl]-N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]acetamide;
N-[(1R)-1-cyclopropyl-2,2,2-trifluoroethyl]-N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]acetamide;
N-[(1S)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
N-[(1R)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
(2R)-2-amino-N-[4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(1S)-4′-({[(1S)-1-cyclopropylethyl](4-fluorophenyl)methyl]carbamoyl}methyl)-3′,5′-dioxo-2,3-dihydrospiro[indene-1,2′-[1,4]ozazolidine-5-yl]azetidine-3-carboxamide;
N-[(1R)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
(2S)-2-amino-N-[4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
(2S)-2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]azetidine-3-carboxamide;
N-benzyl-N-[1-(1-methylazetidin-3-yl)ethyl]-2-{S-[(methylcarbamoyl)amino]-3′,5′-dioxo-2,3-dihydrospiro[indene-1,2′-[1,4]ozazolidine]-4′-yl}acetamide;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(2S)-1, 1,1-trifluoropropan-2-yl]acetamide;
N-[(1R)-3′-(2-{[(1R)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-benzyl-2-[(1R)-5-bromo-2′,4′-dioxo-2,3-dihydro-3′H-spiro[indene-1,5′-[1,3]ozazolidin]-3′-yl]-N-[(1S)-1-cyclopropylethyl]acetamide;
2-[(1S)-5-amino-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-[(1R)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-[(1S)-3′-(2-{[(1R)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-[(1R)-1-cyclopropylethyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1R)-1-cyclopropylethyl]-N-(4-fluorobenzyl)-2-{(1S)-5-[(methylcarbamoyl)amino]-2′,4′-dioxo-2,3-dihydro-3′H-spiro[indene-1,5′-[1,3]ozazolidin]-3′-yl}acetamide;
N-[(1R)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-[(1S)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
tertbutyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]azetidine-1-carboxylate;
2-[(1R)-5-(6-aminopyridin-3-yl)-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-{(1R)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-[(1S)-1-cyclopropylethyl]-N-(4-fluorobenzyl)acetamide;
N-[(1S)-1-cyclopropylethyl]-2-[(1R)-2′,4′-dioxo-5-(pyridin-3-yl)-2,3-dihydro-3′H-spiro[indene-1,5′-[1,3]ozazolidin]-3′-yl]-N-(4-fluorobenzyl)acetamide;
N-benzyl-2-(7-bromo-2′,4′-dioxo-2,3-dihydro-3′H-spiro[chromene-4,5′-[1,3]ozazolidin]-3′-yl)-N-[(1S)-1-cyclopropylethyl]acetamide;
2-[(1R)-5-(azetidin-3-yl)-2′,4′-dioxo-2,3-dihydro-3′H-spiro[indene-1,5′-[1,3 ozazolidin]-3′-yl]-N-benzyl-N-[(1S)-1-cyclopropylethyl]acetamide;
2-[(1R)-3-(1-acetylazetidin-3-yl)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-benzyl-N-[(1S)-1-cyclopropylethyl]-2-[(1R)-5-(1-methylazetidin-3-yl)-2′,4′-dioxo-2,3-dihydro-3′H-spiro[indene-1,5′-[1,3]ozazolidin]-3′-yl]acetamide;
3-[(1R)-3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]-N-methylazetidine-1-carboxamide;
N-benzyl-2-{(1R)-5-[1-(cyclopropylcarbonyl)azetidin-3-yl]-2′,4′-dioxo-2,3-dihydro-3′H-spiro[indene-1,5′-[1,3]ozazolidin]-3′-yl}-N-[(1S)-1-cyclopropylethyl]acetamide;
N-benzyl-N-[(1S)-1-cyclopropylethyl]-2-{(1R)-5-[1-(2-hydroxy-2-methylpropanoyl)azetidin-3-yl]-2′,4′-dioxo-2,3-dihydro-3′H-spiro[indene-1,5′-[1,3]ozazolidin]-3′-yl}acetamide;
N-benzyl-N-[(1S)-1-cyclopropylethyl]-2-{(1R)-5-[1-(methylsulfonyl)azetidin-3-yl]-2′,4′-dioxo-2,3-dihydro-3′H-spiro[indene-1,5′-[1,3]ozazolidin]-3′-yl}acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]cyclobutanecarboxamide;
3-ethoxy-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-5-oxo-D-prolinamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-5-oxo-L-prolinamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]cyclopropanecarboxamide;
N-(4-fluorobenzyl)-2-{(1R)-5-[(methoxyacetyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(1R)-5-[(ethoxyacetyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-3-methylbutanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylpropanamide;
2-[(1R)-5-(acetylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(7-amino-2′,4′-dioxo-2,3-dihydro-3′H-spiro[chromene-4,5′-[1,3]ozazolidin]-3′-yl)-N-benzyl-N-[(1S)-1-cyclopropylethyl]acetamide;
N-(4-fluorobenzyl)-2-[(1R)-5-{[(2-methoxyethyl)(methyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[(dimethylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-{[methyl(2-methylpropyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(cyclopropylmethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(cyclobutylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-({[(2R)-1-hydroxy-3-methylbutan-2-yl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-({[(2S)-tetrahydrofuran-2-ylmethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-({[(2R)-tetrahydrofuran-2-ylmethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-({[2-(propan-2-yloxy)ethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(cyclopropylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-{[ethyl(methyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(diethylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyrrolidine-1-carboxamide;
2-[(1R)-5-({[2-(dimethylamino)ethyl](methyl)carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N²-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-L-leucinamide;
N-(4-fluorobenzyl)-2-[(1R)-5-{[(2-hydroxyethyl)(propyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(2-methoxyethyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(3-hydroxypropyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-({[(2S)-1-hydroxy-3-methylbutan-2-yl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[methyl(propan-2-yl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(2-cyanoethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-{[methyl(propyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-benzyl-N-[(1S)-1-cyclopropylethyl]-2-{7′-[(methylcarbamoyl)amino]-2′,4′-dioxo-2,3-dihydro-3′H-spiro[chromene-4,5′-[1,3]ozazolidin]-3′-yl}acetamide;
2-[7-(acetylamino)-2′,4′-dioxo-2,3-dihydro-3′H-spiro[chromene-4,5′-[1,3]ozazolidin]-3′-yl]-N-benzyl-N-[(1S)-1-cyclopropylethyl]acetamide;
N-[(1S)-1-cyclopropylethyl]-2-[(1R)-2′,4′-dioxo-5-(pyridin-4-yl)-2,3-dihydro-3′H-spiro[indene-1,5′-[1,3]ozazolidin]-3′-yl]-N-(4-fluorobenzyl)acetamide;
N-[(1S)-1-cyclopropylethyl]-N-(4-fluorobenzyl)-2-[(1R)-5-(6-hydroxypyridazin-3-yl)-2′,4′-dioxo-2,3-dihydro-3′H-spiro[indene-1,5′-[1,3]ozazolidin]-3′-yl]acetamide;
N-[(1S)-1-cyclopropylethyl]-2-[(1R)-2′,4′-dioxo-5-(1H-pyrazol-4-yl)-2,3-dihydro-3′H-spiro[indene-1,5′-[1,3]ozazolidin]-3′-yl]-N-(4-fluorobenzyl)acetamide;
N-benzyl-N-[(1S)-1-cyclopropylethyl]-2-[2′,4′-dioxo-7-(6-oxo-1,6-dihydropyridin-3-yl)-2,3-dihydro-3′H-spiro[chromene-4,5′-[1,3]ozazolidin]-3′-yl]acetamide;
2-{(1R)-2′,4′-dioxo-5-[(tetrahydrofuran-2-ylmethyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[(cyclopentylmethyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[(2,2-dimethylbutyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-2′,4′-dioxo-5-[(tetrahydrofuran-3-ylmethyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-{(1R)-5-[(2-methylpropyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
tert-butyl [(2R)-1-{[3′-(2-{benzyl[{18}-1-cyclopropylethyl]amino}-2-oxoethyl)-2′,4′-dioxo-2,3-dihydrospiro[chromene-4,5′-[1,3]ozazolidin]-7-yl]amino}-3-methyl-1-oxobutan-2-yl]carbamate;
N-[3′-(2-{benzyl[(1S)-1-cyclopropylethyl]amino}-2-oxoethyl)-2′,4′-dioxo-2,3-dihydrospiro[chromene-4,5′-[1,3]ozazolidin]-7-yl]-D-valinamide;
2-[(1R)-6-amino-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
2-[(1R)-6-(acetylamino)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-benzyl-N-[(1S)-1-cyclopropylethyl]-2-{(1R)-6-[(methylcarbamoyl)amino]-2′,4′-dioxo-2,3-dihydro-3′H-spiro[indene-1,5′-[1,3]ozazolidin]-3′-yl}acetamide;
2-[(1S)-6-amino-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
2-[(1S)-6-(acetylamino)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-benzyl-N-[(1S)-1-cyclopropylethyl]-2-{(1S)-6-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-2-[(1R)-2′,4′-dioxo-5-(pyridin-2-yl)-2,3-dihydro-3′H-spiro[indene-1,5′-[1,3]ozazolidin]-3′-yl]-N-(4-fluorobenzyl)acetamide;
N-[(1S)-1-cyclopropylethyl]-N-(3-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropyl-2,2,2-trifluoroethyl]-N-(4-fluorobenzyl)-2-{7′-[(methylcarbamoyl)amino]-2,4-dioxo-2′,3′-dihydro-3′H-spiro[1,3-ozazolidine-5,4′-thiochromen]-3-yl}acetamide;
2-[(1R)-5-(carbamoylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1S)-1-cyclopropyl-2,2,2-trifluoroethyl]-N-(4-fluorobenzyl)-2-{7′-[(methylcarbamoyl)amino]-1′,1′-dioxido-2,4-dioxo-2′,3′-dihydro-3′H-spiro[1,3-ozazolidine-5,4′-thiochromen]-3-yl}acetamide;
N-(4-fluorobenzyl)-2-((R)-5-(3-(N-methylsulfamoyl)ureido)-2′,4′-dioxo-2,3-dihydrospiro[indene-1,5′-ozazolidine]-3-yl)-N—((S)-1,1,1-trifluoropropan-2-yl)acetamide
methyl {[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}sulfamate;
2-((R)-5-(3-(N-(cyclopropylmethyl)sulfamoyl)ureido)-2′,4′-dioxo-2,3-dihydrospiro[indene-1,5′-ozazolidine]-3′-yl)-N-(4-fluorobenzyl)-N—((S)-1,1,1-trifluoropropan-2-yl)acetamide (1R)-3′-(2-{benzyl[(1S)-1-cyclopropylethyl]amino}-2-oxoethyl)-N-(2-methylpropyl)-2′,4′-dioxo-2,3-dihydrospiro[indene-1,5′-[1,3]ozazolidine]-5-carboxamide;
N-[(1S)-1-cyclopropylethyl]-N-(2-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,3-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-chloro-4-fluorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3-chloro-4-fluorobenzyl)-N-(1S)-1-cyclopropylethyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-[4-(trifluoromethyl)benzyl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1-methyl-1H-pyrazole-4-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1-methyl-1H-pyrazole-3-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-4-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-2-carboxamide;
2-(5-bromo-4-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
2-(5-bromo-6-fluoro-2′,4′-dioxo-2,3-dihydro-3′H-spiro[indene-1,5′-[1,3]ozazolidin]-3′-yl) -N-[(1S)-1-cyclopropyl-2,2,2-trifluoroethyl]-N-(4-fluorobenzyl)acetamide;
(1R)-3′-(2-{benzyl[(1S)-1-cyclopropylethyl]amino}-2-oxoethyl)-N-[(2R)-1-hydroxy-3-methylbutan-2-yl]-2′,4′-dioxo-2,3-dihydrospiro[indene-1,5′-[1,3]ozazolidine]-5-carboxamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-[(5-methyl-1,3,4-thiadiazol-2-yl)methyl]acetamide;
2-(5-amino-4-fluoro-2′,4′-dioxo-2,3-dihydro-3′H-spiro[indene-1,5′-[1,3]ozazolidin]-3′-yl]-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{4-fluoro-5-[(methylcarbamoyl)amino]-2′,4′-dioxo-2,3-dihydro-3′H-spiro[indene-1,5′-[1,3]ozazolidin]-3′-yl}acetamide;
N-(4-cyanobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3-methylbenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,4-dichlorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-methoxybenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3-methoxybenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-methylbenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-[(1R)-5-{[(1-methyl-1H-pyrazol-4-yl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{7′-[1-(2-amino-2-oxoethyl)-1H-pyrazol-4-yl]-2′,4′-dioxo-2,3-dihydro-3′H-spiro[chromene-4,5′-[1,3]ozazolidin]-3′-yl}-N-benzyl-N-[(1S)-1-cyclopropylethyl]acetamide;
2-{(1R)-2′,4′-dioxo-5-[(phenylcarbamoyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-2′,4′-dioxo-5-[(pyridin-3-ylcarbamoyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-(5-amino-6-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{6-fluoro-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetamide;
N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(dimethylamino)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(4-methylpyrimidin-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[5-(difluoromethyl)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(6-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2′-yl]acetamide;
N-(4-fluorobenzyl)-2-{(1R)-5-[1-(2-methylpropyl)-1H-pyrazol-4-yl]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(1R)-5-[4-(difluoromethyl)pyridin-2-yl]-2′,4′-dioxo-2,3-dihydro-3′H-spiro[indene-3,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(3-fluoropyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(3,3-thiazol-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-N-methylbenzamide;
2-{(3R)-5-[2-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(5-cyanopyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(1-methyl-1H-1,2,4-triazol-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(5-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-(6-cyanopyridin-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(5-fluoro-3-methylpyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(5-methyl]-1,3,4-thiadiazol-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(4-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(6-methoxypyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[2-(dimethylamino)pyrimidin-5-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(acetylamino)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(1-methyl-1H-1,2,4-triazol-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(1-methyl-1H-pyrazol-4-yl)-2′,4′-dioxo-2,3-dihydro-3′H-spiro[idene-1,5′-[1,3]ozazolidin]-3′-yl]-N-[(2S)-1,1,1-trifluoropropan-2-yl]acetamide;
N-cyclopropyl-5-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]furan-2-carboxamide;
N-(4-fluorobenzyl)-2-[(1R)-5-(5-fluoropyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
4-fluoro-3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
5-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-N-methylpyridine-2-carboxamide;
6-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-3-carboxamide;
N-(4-fluorobenzyl)-2-[(1R)-5-(2-methoxypyrimidin-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
6-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-ylpyridine-2-carboxamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]thiophene-3-carboxamide;
2-{(3R)-5-[4-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(1,3-dimethyl-2,4-dioxo-1,2,3,4-tetrahydropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4-cyanopyridin-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-4-carboxamide;
2-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
2-{(1R)-5-[3-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(6-cyano-5-methoxypyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(1-methyl-1H-pyrazol-3-yl)-2′,4′-dioxo-2,3-dihydro-3′H-spiro[indene-1,5′-[1,3]ozazolidin]-3′-yl]-N-[(2R)-1,1,1-trifluoropropan-2-yl]acetamide;
2-[(1R)-2′,4′-dioxo-5-(1,3,5-trimethyl-1H-pyrazol-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(3,4-difluorophenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2,5-difluoro-4-methoxyphenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyrimidin-5-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-2′,4′-dioxo-5-[5-(trifluoromethyl)pyridin-3-yl]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-2′,4′-dioxo-5-(1,3-thiazol-2-yl)-2,3-dihydro-3′H-spiro[idene-1,5′-[1,3]ozazolidin]-3′-yl]-N-(4-fluorobenzyl)-N-[(2S)-1,1,1-trifluoropropan-2-yl]acetamide;
2-[(1R)-5-(2-cyanopyridin-4-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyrazin-2-yl)-2,3-dihydro-3′H-spiro[idene-1,5′-[1,3]ozazolidin]-3′-yl]-N-(4-fluorobenzyl)-N-[(2S)-1,1,1-trifluoropropan-2-yl]acetamide;
2-{(1R)-5-[4-(carbamoylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(2-cyanopyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(6-cyano-5-fluoropyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
2-[(1R)-5-{[(4-aminophenyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-{6-[(methylcarbamoyl)amino]-2′,4′-dioxo-3,4-dihydro-2H,3′H-spiro[napthalene-1,5′-[1,3]ozazolidin]-3′-yl}-N-[(2S)-1,1,1-trifluoropropan-2-yl]acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(pyrimidin-5-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(pyrimidin-2-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-thiazol-5-ylmethyl)acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N²-(4-fluorobenzyl)-N²-({(1R)-5-[(methylcarbamoyl)amino]-2′,4′-dioxo-2,3-dihydro-3′H-spiro[indene-[1,5′-[1,3]ozazolidin]-3′-yl}acetyl)-L-alaninamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1H-pyrazole-5-carboxamide;
N-(4-fluorobenzyl)-2-[(1R)-5-{[(methylsulfonyl)acetyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(methylsulfonyl)acetyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(dimethylsulfamoyl)acetyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-2′,4′-dioxo-5-[(sulfamoylacetyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidine-1-carboxamide
N-benzyl-N-ethyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
methyl N′-cyano-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamimidothioate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanamide;
N-(4-fluorobenzyl)-2-{(1R)-5-[(methylcarbamothioyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(tetrahydro-2H-pyran-4-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-oxazol-4-ylmethyl)acetamide;
2-[(1R)-5-(5-cyanothiophen-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4-cyano-3-fluorophenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(5-cyanothiophen-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(N″-cyano-N′-methylcarbamimidamido)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4,6-difluoropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[2-(acetylamino)-5-methylpyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(5-fluoropyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(furan-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(2-oxo-2,3-dihydro-1H-benzimidazol-5-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(4-methylpyrimidin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-2′,4′-dioxo-5-[1-(tetrahydro-2H-pyran-2-yl)-1H-pyrazol-3-yl]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(5-fluoro-6-methylpyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[6-(difluoromethyl)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(difluoromethyl)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(furan-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[1-(ethoxymethyl)-1H-imidazol-2-yl]-2′,4′-dioxo-2,3-dihydro-3′H-spiro[indene-[1,3]ozazolidin]-3′-yl}-N-(4-fluorobenzyl)-N-[(2S)-1,1,1-trifluoropropan-2-yl]acetamide;
2-[(1R)-5-(5-cyano-1,2-dimethyl-6-oxo-1,6-dihydropyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]thiophene-2-carboxamide;
2-[(1R)-5-(2,6-dioxo-1,2,5,6-tetrahydropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2,4-dimethyl-1,3-thiazol-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[1-(cyanomethyl)-3,5-dimethyl-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-yl]acetamide;
2-{(1R)-5-[3-(carbamoylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-[3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)-2,2-dimethylpropyl]propanamide;
(1R)-3′-(2-{benzyl[(1S)-1-cyclopropylethyl]amino}-2-oxoethyl)-N-methyl-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(propan-2-yl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(2-methoxyethyl)-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(tetrahydrofuran-2-ylmethyl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(tetrahydrofuran-3-ylmethyl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(cyanomethyl)-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(3-hydroxypropyl)-2′,4′-dioxo-2,3-dihydrospiro[indene-1,5′-[1,3]ozazolidine]-5-carboxamide;
3′-{2-[benzyl(1-cyclopropylethyl)amino]-2-oxoethyl}-N-(1-hydroxy-3-methylbutan-2-yl)-2′,4′-dioxo-2,3-dihydrospiro[indene-1,5′-[1,3]ozazolidine]-5-carboxamide;
N-(1-amino-4-methyl-1-oxopentan-2-yl)-3′-{2-[benzyl(1-cyclopropylethyl)amino]-2-oxoethyl}-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-[2-(diethylamino)ethyl]-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-[3-(1H-imidazol-1-yl)propyl]-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-[3-(2-oxopyrrolidin-1-yl)propyl]-2,3-dihydrospiro[indene-1,5′-[1,3]ozazolidine]-5-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]tetrahydropyrimidine-1(2H)-carboxamide;
3-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]tetrahydropyrimidine-1(2H)-carboxamide;
N-(4-fluorobenzyl)-2-{(1R)-5-[1-(2-hydroxyethyl)-1H-pyrazol-4-yl]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-phenylacetamide;
N-benzyl-2-{(1R)-5-[(methylcarbamoyl)amino]-2′,4′-dioxo-2,3-dihydro-3′H-spiro[indene-[1,5′-[1,3]ozazolidin]-3′-yl}-N-[(1S)-1-phenylethyl]acetamide;
N,N-dibenzyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
(2R)-3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-hydroxy-2-methylpropanamide;
(2S)-3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-hydroxy-2-methylpropanamide;
2-[(1R)-2′,4′-dioxo-5-({[3-(pyrrolidin-1-ylmethyl)phenyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-({[3-(morpholin-4-ylmethyl)phenyl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N³-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-N-methyl-beta-alaninamide;
N-[3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)phenyl](propanamide;
2-(6-bromo-2′,4,4′-trioxo-3,4-dihydro-2H,3′H-spiro[naphthalene-1,5′-[1,3]ozazolidin]-3′-yl)-N-[(1S)-1-cyclopropyl-2,2,2-trifluoroethyl]-N-(4-fluorobenzyl)acetamide;
N-(2-chlorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)acetamide;
N-[(1S)-1-cyclopropyl-2,2,2-trifluoroethyl]-N-(4-fluorobenzyl)-2-{6-[(methylcarbamoyl)amino]-2′,4,4′-trioxo-3,4-dihydro-2H,3′H-spiro[naphthalene-1,5′-[1,3]ozazolidin]-3′-yl}acetamide;
N-(4-fluorobenzyl)-2-{(1S)-6-[(methylcarbamoyl)amino]-2′,4′-dioxo-3,4-dihydro-2H,3′H-spiro[naphthalene-1,5′-[1,3]ozazolidin]-3′-yl}-N-[(2S)-1,1,1-trifluoropropan-2-yl]acetamide;
N-(4-fluorobenzyl)-2-{(1R)-6-[(methylcarbamoyl)amino]-2′,4′-dioxo-3,4-dihydro-2H,3′H-spiro[naphthalene-1,5′-[1,3]ozazolidin]-3′-yl}-N-[(2S)-1,1,1-trifluoropropan-2-yl]acetamide;
2-[(1R)-5-(4,5-dihydro-1H-imidazol-2-ylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(6-amino-2′,4′-dioxo-3,4-dihydro-2H,3′H-spiro[naphthalene-1,5′-[1,3]ozazolidin]-3′-yl)-N-[(1S)-1-cyclopropyl-2,2,2-trifluoroethyl]-N-(4-fluorobenzyl)acetamide;
2-[(1R)-5-({[3-(acetylamino)propyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-({[2-(acetylamino)ethyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
tert-butyl [3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)-2,2-dimethylpropyl]carbamate;
N-[2-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)ethyl]propanamide;
2-[(1R)-5-{[(3-amino-2,2-dimethylpropyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-({[3-(acetylamino)phenyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
methyl 2-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]amino}-4,5-dihydro-1H-imidazole-1-carboxylate;
2-{5-bromo-6-[(methylcarbamoyl)amino]-2′,4′-dioxo-3,4-dihydro-2H,3′H-spiro[naphthalene-1,5′-[1,3]ozazolidin]-3′-yl}-N-[(1S)-1-cyclopropyl-2,2,2-trifluoroethyl]-N-(4-fluorobenzyl)acetamide;
N-cyclohexyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-cyclopentyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropyl-2,2,2-trifluoroethyl]-N-(4-fluorobenzyl)-2-{4-hydroxy-6-[(methylcarbamoyl)amino]-2′,4′-dioxo-3,4-dihydro-2H,3′H-spiro[naphthalene-1,5′-[1,3]ozazolidin]-3′-yl}acetamide;
N-benzyl-N-[1-(furan-2-yl)ethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-cyclobutyl-2-{(1R)-5-[(methylcarbamoyl)amino]-2′,4′-dioxo-2,3-dihydro-3′H-spiro[indene-1,5′-[1,3]ozazolidin]-3-yl}acetamide;
N-[(1S)-1-cyclopropyl-2,2,2-trifluoroethyl]-N-(4-fluorobenzyl)-2-{4-fluoro-6-[(methylcarbamoyl)amino]-2′,4′-dioxo-3,4-dihydro-2H,3′H-spiro[naphthalene-1,5′-[1,3]ozazolidin]-3′-yl}acetamide;
N-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-beta-alanine;
N-cyclopropyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl N-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl}carbamoyl]-beta-alaninate;
N-(3,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(4-fluorobenzyl)-N-(3-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(1S)-5-amino-2′,4′-dioxo-2,3-dihydro-3′H-spiro[indene-1,5′-[1,3]ozazolidin]-3′-yl]-N-[(1R)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
ethyl ({(3R)-3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(3R)-piperidin-3-yl]acetamide;
ethyl ({(3R)-3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]piperidin-1-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(methylsulfonyl)piperidin-4-yl]acetamide;
N-(4-fluorobenzyl)-2-{6-fluoro-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-(5-amino-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1-phenylethyl)acetamide;
2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)-N-[2-(trifluoromethyl)benzyl]acetamide;
2-(5-bromo-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
N-benzyl-N-(cyanomethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-(2-cyanoethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(methylsulfonyl)propan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(1-methyl-1H-pyrazol-4-yl)methyl]acetamide;
N-[(1S)-1-cyclopropylethyl]-N-(4-fluorobenzyl)-2-{4-methoxy-5-[(methylcarbamoyl)amino]-2′,4′-dioxo-2,3-dihydro-3′H-spiro[indene-1,5′-[1,3]ozazolidin]-3′-yl}acetamide;
2-[5-(acetylamino)-4-methoxy-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-[(1R)-5-({[3-(acetylamino)-2,2-dimethylpropyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclopentyl-2-{(1R)-5-[(methyl carbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(pyridin-3-yl)acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1-methyl-1H-pyrazol-4-yl)acetamide;
N-benzyl-N-(2,2-difluorocyclopentyl)-2-{5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-(2-methylcyclopropyl)acetamide;
N-(2,2-dimethylcyclopentyl)-N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-(2-methylcyclopentyl)acetamide;
2-(5-{[(2-cyanoethyl)carbamoyl]amino}-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
methyl [(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamate;
4-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)butanoic acid;
N²-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylalaninamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylalaninamide;
N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
N-cyclopentyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-oxazol-4-ylmethyl)acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-[1-(pyridin-2-yl)ethyl]acetamide;
N-(4-chlorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3,5-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-(5-{[(cyanomethyl)carbamoyl]amino}-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
2-[(1R)-5-{[(cyanomethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclohexyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-pyrrolidin-3-yl]acetamide;
N-(4-fluorobenzyl)-N-(trans-3-hydroxycyclobutyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-1-(methylsulfonyl)pyrrolidin-3-yl]acetamide;
ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidin-1-yl}sulfonyl)carbamate;
N-(1-cyanopropan-2-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
5-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)pentanoic acid;
N-[(1S)-1-cyclopropyl-2,2,2-trifluoroethyl]-N-(4-fluorobenzyl)-2-[4-fluoro-5-(1-methyl-1H-pyrazol-4-yl)-2′,4′-dioxo-2,3-dihydro-3′H-spiro[indene-1,5′-[1,3]ozazolidin]-3′-yl]acetamide;
N-(4-fluorobenzyl)-2-[6-fluoro-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-[(3R)-1-acetylpyrrolidin-3-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-1-(2-sulfamoylethyl)pyrrolidin-3-yl]acetamide;
N-(4-fluorobenzyl)-N-(trans-4-hydroxycyclohexyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
methyl (1R,3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-3-(methylsulfonyl)tetrahydropyrimidine-1(2H)-carboxamide;
N²-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]glycinamide;
2-{(1R)-5-[(methylcarbamoyl)amino]-2′,4′-dioxo-2,3-dihydro-3′H-spiro[indene-1,5′-[1,3]ozazolidin]-3′-yl}-N-[1-(2-methylphenyl)ethyl]-N-(2-methylpropyl)acetamide;
N-(2-chlorobenzyl)-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)acetamide;
N-(4-fluorobenzyl)-N-(4-methoxyphenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(4-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(2-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(trifluoromethyl)cyclopropyl]acetamide;
2-[(1R)-5-(3,4-dihydro-2H-pyrrol-5-ylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(3,4-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3-fluorobenzyl)-2-{5-[(methylcarbamoyl)amino]2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(2,5-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3-chlorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{5-[1-(2-amino-2-oxoethyl)-1H-pyrazol-4-yl]-6-fluoro-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(3R)-pyrrolidin-3-yl]acetamide;
ethyl ({(3R)-3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]pyrrolidin-1-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(piperidin-4-yl)acetamide;
ethyl ({4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]azetidine-1-carboxylate;
N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}-N-[2-(trifluoromethyl)benzyl]acetamide;
N-(2-chloro-4-fluorobenzyl)-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(3-methoxyphenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-(pyridin-4-yl)acetamide;
N-(4-fluorobenzyl)-2-(6-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl)-N-[(2S)-1,1,1-trifluoropropan-2-yl]acetamide;
methyl (1S,3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(tetrahydro-2H-pyran-4-yl)acetamide;
N-(1,1-dioxidotetrahydro-2H-thiopyran-4-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2,2-difluorocyclopentyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-cyclohexyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-[(1S)-1-cyclopropyl-2,2,2-trifluoroethyl]-N-(4-fluorobenzyl)-2-[7-(1-methyl-1H-pyrazol-4-yl)-2′,4′-dioxo-1H,3′H-spiro[isochromene-4,5′-[1,3]ozazolidin]-3′-yl]acetamide;
2-(7-amino-2′,4′-dioxo-1H,3′H-spiro[isochromene-4,5′-[1,3]ozazolidin]-3′-yl)-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{7-[(methylcarbamoyl)amino]-2′,4′-dioxo-1H,3′H-spiro[isochromene-4,5′-[1,3]ozazolidin]-3′-yl}acetamide;
N-(4-fluorobenzyl)-2-{7-[(methylcarbamoyl)amino]-2′,4′-dioxo-1H,3′H-spiro[isochromene-4,5′-[1,3]ozazolidin]-3′-yl}-N-[(2 S)-1,1,1-trifluoropropan-2-yl]acetamide;
N-(4-fluorobenzyl)-2-[7-(1-methyl-1H-pyrazol-4-yl)-2′,4′-dioxo-1H,3′H-spiro[isochromene-4,5′-[1,3]ozazolidin]-3′-yl]-N-[(2S)-1,1,1-trifluoropropan-2-yl]acetamide;
N-(4-fluorobenzyl)-2-(7-{1-[2-(methylamino)-2-oxoethyl]-1H-pyrazol-4-yl}-2′,4′-dioxo-1H,3′H-spiro[isochromene-4,5′-[1,3]ozazolidin]-3′-yl)-N-[(2S)-1,1,1-trifluoropropan-2-yl]acetamide;
N-(4-fluorobenzyl)-N-{1-[2-(methylamino)-2-oxoethyl]-1H-pyrazol-4-yl}-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[1-(cyanomethyl)-1H-pyrazol-4-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl 3-{1-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]ethyl}azetidine-1-carboxylate;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylic acid;
ethyl ({(3S)-3-[(4-fluorobenzyl) {[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
ethyl ({(3S)-3-[{[(1R)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}(4-fluorobenzyl)amino]pyrrolidin-1-yl}sulfonyl)carbamate;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}acetate;
ethyl ({(3R)-3-[(4-fluorobenzyl) {[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]piperidin-1-yl}sulfonyl)carbamate;
ethyl ({(3R)-3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]piperidin-1-yl}sulfonyl)carbamate;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}acetic acid;
N-(4-fluorobenzyl)-2-{(4S)-7-[(methylcarbamoyl)amino]-2′,4′-dioxo-1H,3′H-spiro[isochromene-4,5′-[1,3]ozazolidin]-3′-yl}-N-[(2 S)-1,1,1-trifluoropropan-2-yl]acetamide;
N-(4-fluorobenzyl)-2-{(4R)-7-[(methylcarbamoyl)amino]-2′,4′-dioxo-1H,3′H-spiro[isochromene-4,5′-[1,3]ozazolidin]-3′-yl}-N-[(2 S)-1,1,1-trifluoropropan-2-yl]acetamide;
tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
tert-butyl (3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-piperidin-3-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3S)-piperidin-3-yl]acetamide;
N-[4-amino-3-(hydroxymethyl)butan-2-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({(3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzamide;
tert-butyl 2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(pyrrolidin-2-ylmethyl)acetamide;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexylidene}acetate;
N-ethyl-4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxamide;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexylidene}acetic acid;
N-(4-fluorobenzyl)-2-[(1R)-5-(oxetan-3-ylamino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzamide;
N-[(1-acetylpyrrolidin-2-yl)methyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{[1-(methylsulfonyl)pyrrolidin-2-yl]methyl}acetamide;
2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}-N-methylpyrrolidine-1-carboxamide;
N-(3,4-difluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{[1-(methylsulfamoyl)pyrrolidin-2-yl]methyl}acetamide;
N-(cyclopropylmethyl)-N-(2,3-dihydro-1H-inden-1-yl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexyl}acetic acid;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-3-methylazetidin-1-yl}sulfonyl)carbamate;
N-(3-cyanophenyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
tert-butyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}carbamate;
N-(3-aminocyclobutyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[3-(acetylamino)cyclobutyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{3-[(methylsulfonyl)amino]cyclobutyl}acetamide;
2,2′-({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}imino)diacetic acid;
N-{3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}glycine;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxylate;
methyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}carbamate;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}sulfamoyl)carbamate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxylic acid;
ethyl ({4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]piperidin-1-yl}sulfonyl)carbamate;
2-[(1 S,3S)-5-bromo-3-hydroxy-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(5-chloro-4-cyano-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
benzyl 3-{[(4-fluorobenzyl)({(1R)-5-[(methylcarbamoyl)amino]-2′,4′-dioxo-2,3-dihydro-3′H-spiro[indene-,5′-[1,3]ozazolidin]-3′-yl}acetyl)amino]methyl}azetidine-1-carboxylate;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoate;
N-(4-fluorobenzyl)-2-[(1 S,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
ethyl ({3,3-difluoro-4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
ethyl ({3,3-difluoro-4-[(4-fluorobenzyl){[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
N-(4-fluorobenzyl)-2-[(1S)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1S)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-N-(3-methoxycyclobutyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-N-methylcyclobutanecarboxamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxamide;
2-(5-amino-4-cyano-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetic acid;
(2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidin-1-yl)acetic acid;
methyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetate;
{3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetic acid;
tert-butyl {4-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]cyclohexylidene}acetate;
tert-butyl {4-[(4-fluorobenzyl) {[(1S)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetate;
{4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetic acid;
N-(4-fluorobenzyl)-2-[(1 S,3R)-3-fluoro-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(azetidin-3-ylmethyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
{4-[{[(1R)-2′,4′-dioxo-2,3-dihydro-3′H-spiro[indene-1,5′-[1,3]ozazolidin]-3′-yl]acetyl}(4-fluorobenzyl)amino]cyclohexylidene}acetic acid;
tert-butyl {trans-4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexyl}carbamate;
N-(trans-4-aminocyclohexyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{trans-4-[(methylsulfonyl)amino]cyclohexyl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(trans-4-{[(trifluoromethyl)sulfonyl]amino}cyclohexyl)acetamide;
tert-butyl 6-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-2-azaspiro[3.3]heptane-2-carboxylate;
N-(2-azaspiro[3.3]hept-6-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(1 S,3R)-5-bromo-3-hydroxy-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1-acetylazetidin-3-yl)methyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-[(2S)-1,1,1-trifluoropropan-2-yl]-2-(2′,3,4′-trioxo-1,2,3,4,8,9-hexahydro-3′H-spiro[cyclopenta[f]quinazoline-7,5′-[1,3]ozazolidin]-3′-yl)acetamide;
ethyl [(3-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}azetidin-1-yl)sulfonyl]carbamate;
ethyl [(2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidin-1-yl)sulfonyl]carbamate;
ethyl ({6-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-2-azaspiro[3.3]hept-2-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(3-{[(trifluoromethyl)sulfonyl]amino}cyclobutyl)acetamide;
tert-butyl 3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]benzoate;
tert-butyl 3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]benzoate;
3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]benzoic acid;
N-(4-fluorobenzyl)-2-[(1S)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(azetidin-3-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]azetidin-1-yl}sulfonyl)carbamate;
2-[(1S)-5-bromo-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1 S,3R)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-[(1S)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-D-valinamide;
N-benzyl-N-[(1S)-1-cyclopropylethyl]-2-[2′,4′-dioxo-5-(2H-tetrazol-5-yl)-2,3-dihydro-3′H-spiro[indene-1,5′-[1,3]ozazolidin]-3′-yl]acetamide;
2,2′-({[3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]carbonyl}imino)diacetic acid;
N-(4-fluorobenzyl)-2-[(1 S,3R)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1 S,3R)-5-[1-(2-amino-2-oxoethyl)-1H-pyrazol-4-yl]-3-hydroxy-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1S)-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
methyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanoate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanoic acid;
2-{(1S)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R,3R)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R,3R)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1 S,3S)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1S)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1 S,3R)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyridin-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(pyridin-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(pyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(6-oxo-1,6-dihydropyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(6-oxo-1,6-dihydropyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-phenyl-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-phenyl-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclohexyl-N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-(tetrahydro-2H-pyran-4-yl)acetamide;
3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclobutanecarboxylic acid;
4-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexanecarboxylic acid;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-{trans-4-[(methylsulfonyl)amino]cyclohexyl}acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-(3-{[(trifluoromethyl)sulfonyl]amino}cyclobutyl)acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-[1-(methylsulfonyl)piperidin-4-yl]acetamide;
2-[(1R,3S)-5-bromo-3-hydroxy-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R,3S)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2,2′-({3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclobutyl}imino)diacetic acid;
N-(4-fluorobenzyl)-2-[(1 S,3R)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(1,2-oxazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1S)-5-(1,2-oxazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1 S,3R)-3-fluoro-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-[(1R,3S)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R,3S)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
{4-[(4-fluorobenzyl) {[(1 S,3R)-3-fluoro-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetic acid;
N-(4-fluorobenzyl)-2-[(1R,3R)-3-fluoro-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R,3R)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide; and pharmaceutically acceptable salts thereof.

Still another embodiment pertains to compounds of Formula (XIb), selected from the group consisting of:
N-benzyl-N-(1-cyclobutylethyl)-2-(2,5-dioxo-2′,3′-dihydrospiro[imidazolidine-4,1′-indene]-1-yl)acetamide;
N-benzyl-N-(1-cyclopropylethyl)-2-(2,5-dioxo-3′,4′-dihydro-2′H-spiro[imidazolidine-4,1′-naphthalen]-1-yl)acetamide;
N-benzyl-N-(1-cyclopropylethyl)-2-(2′,5′-dioxospiro[chroman-4,4′-imidazolidine]-1′-yl)acetamide;
N-benzyl-N-(1-cyclopropylethyl)-2-(2,2′,5-trioxospiro[imidazolidine-4,3′-indoline]-1-yl)acetamide;
N-(4-bromobenzyl)-N-(1-cyclopropylethyl)-2-(2,5-dioxo-2′,3′-dihydrospiro[imidazolidine-4,1′-indene]-1-yl)acetamide;
N-(1-cyclopropylethyl)-2-(2,5-dioxo-2′,3′-dihydrospiro[imidazolidine-4,1′-indene]-1-yl)-N-(furan-2-ylmethyl)acetamide;
N-benzyl-N-(1-cyclopropylethyl)-2-(1′-methyl-2,2′,5-trioxospiro[imidazolidine-4,3′-indoline]-1-yl)acetamide;
N-(1-cyclopropylethyl)-2-(2,5-dioxo-2′,3′-dihydrospiro[imidazolidine-4,1′-indene]-1-yl)-N-(thiophen-2-ylmethyl)acetamide;
N-benzyl-N-(1-cyclopropylethyl)-2-(3-methyl-2,5-dioxo-2′,3′-dihydrospiro[imidazolidine-4,1′-indene]-1-yl)acetamide;
N-(1-cyclopropylethyl)-2-(2; 5-dioxo-2′,3′-dihydrospiro[imidazolidine-4,1′-indene]-1-yl)-N-(4-fluorobenzyl)acetamide;
N-(1-cyclopropylethyl)-2-(2,5-dioxo-2′,3′-dihydrospiro[imidazolidine-4,1′-indene]-1-yl)-N-(2-fluorobenzyl)acetamide;
N-(1-cyclopropylethyl)-2-(2,5-dioxo-2′,3′-dihydrospiro[imidazolidine-4,1′-indene]-1-yl)-N-(3-fluorobenzyl)acetamide;
N-benzyl-2-(5′-bromo-2,5-dioxo-2′,3′-dihydrospiro[imidazolidine-4,1′-indene]-1-yl)-N-(1-cyclopropylethyl)acetamide;
2-(5′-bromo-2,5-dioxo-2′,3′-dihydrospiro[imidazolidine-4,1′-indene]-1-yl)-N-(1-cyclopropylethyl)-N-(furan-2-ylmethyl)acetamide;
N-benzyl-N-(1-cyclopropylethyl)-2-(5′-fluoro-2,5-dioxo-2′,3′-dihydrospiro[imidazolidine-4,1′-indene]-1-yl)acetamide;
N-benzyl-2-(4′-bromo-2,5-dioxo-2′,3′-dihydrospiro[imidazolidine-4,1′-indene]-1-yl)-N-(1-cyclopropylethyl)acetamide;
N-(1-cyclopropylethyl)-2-(2,5-dioxo-2′,3′-dihydrospiro[imidazolidine-4,1′-indene]-1-yl)-N-(pyridin-3-ylmethyl)acetamide;
N-(1-cyclopropylethyl)-2-(2,5-dioxo-2′,3′-dihydrospiro[imidazolidine-4,1′-indene]-1-yl)-N-(pyridin-2-ylmethyl)acetamide;
N-benzyl-N-(1-cyclopropylethyl)-2-(5′-methoxy-2,5-dioxo-2′,3′-dihydrospiro[imidazolidine-4,1′-indene]-1-yl)acetamide;
N-benzyl-N-(1-cyclopropylethyl)-2-(4′-methoxy-2,5-dioxo-2′,3′-dihydrospiro[imidazolidine-4,1′-indene]-1-yl)acetamide;
N-benzyl-2-(5′-cyano-2,5-dioxo-2′,3′-dihydrospiro[imidazolidine-4,1′-indene]-1-yl)-N-(1-cyclopropylethyl)acetamide;
N-(1-cyclopropylethyl)-2-(2,5-dioxo-2′,3′-dihydrospiro[imidazolidine-4,1′-indene]-1-yl)-N-(pyridin-4-ylmethyl)acetamide;
N-benzyl-2-(4′-chloro-2,5-dioxo-2′,3′-dihydrospiro[imidazolidine-4,1′-indene]-1-yl)-N-(1-cyclopropylethyl)acetamide;
N-benzyl-2-(2,5-dioxo-2′,3′-dihydrospiro[imidazolidine-4,1′-indene]-1-yl)-N-isopropylacetamide;
N-benzyl-N-sec-butyl-2-(2,5-dioxo-2′,3′-dihydrospiro[imidazolidine-4,1′-indene]-1-yl)acetamide;
N-(1-cyclobutylethyl)-2-(2,5-dioxo-2′,3′-dihydrospiro[imidazolidine-4,1′-indene]-1-yl)-N-(4-fluorobenzyl)acetamide;
N-benzyl-2-(4′-bromo-2,5-dioxo-2′,3′-dihydrospiro[imidazolidine-4,1′-indene]-1-yl)-N—((R)-1-cyclobutylethyl)acetamide;
2-(4′-bromo-2,5-dioxo-2′,3′-dihydrospiro[imidazolidine-4,1′-indene]-1-yl)-N-(1-cyclopropylethyl)-N-(4-fluorobenzyl)acetamide;
N-benzyl-2-(5′-bromo-2,5-dioxo-2′,3′-dihydrospiro[imidazolidine-4,1′-indene]-1-yl)-N-(1-cyclobutylethyl)acetamide;
2-(5′-bromo-2,5-dioxo-2′,3′-dihydrospiro[imidazolidine-4,1-indene]-1-yl)-N-(1-cyclopropylethyl)-N-(4-fluorobenzyl)acetamide;
1-(2-(benzyl(1-cyclopropylethyl)amino)-2-oxoethyl)-2,5-dioxo-2′,3′-dihydrospiro[imidazolidine-4,1′-indene]-5′-carboxamide;
N-benzyl-2-(4′-cyano-2,5-dioxo-2′,3′-dihydrospiro[imidazolidine-4,1′-indene]-1-yl)-N-(1-cyclopropylethyl)acetamide;
1-(2-(benzyl(1-cyclopropylethyl)amino)-2-oxoethyl)-2,5-dioxo-2′,3′-dihydrospiro[imidazolidine-4,1′-indene]-4′-carboxamide;
N-(1-cyclopropylethyl)-2-(2,5-dioxo-2′,3′-dihydrospiro[imidazolidine-4,1′-indene]-1-yl)-N-(naphthalen-2-ylmethyl)acetamide;
N-((5-bromofuran-2-yl)methyl)-N-(1-cyclopropylethyl)-2-(2,5-dioxo-2′,3′-dihydrospiro[imidazolidine-4,1′-indene]-1-yl)acetamide;
N-benzyl-N—((S)-1-cyclobutylethyl)-2-((S)-2,5-dioxo-2′,3′-dihydrospiro[imidazolidine-4,1′-indene]-1-yl)acetamide;
N-benzyl-N—((R)-1-cyclobutylethyl)-2-((S)-2,5-dioxo-2′,3′-dihydrospiro[imidazolidine-4,1′-indene]-1-yl)acetamide;
N-((1H-indol-5-yl)methyl)-N-(1-cyclopropylethyl)-2-(2,5-dioxo-2′,3′-dihydrospiro[imidazolidine-4,1′-indene]-1-yl)acetamide;
N-benzyl-2-(2,5-dioxo-2′,3′-dihydrospiro[imidazolidine-4,1′-indene]-1-yl)-N-(pentan-3-yl)acetamide;
N-benzyl-N-(1-cyclopropylethyl)-2-(3′-methoxy-2,5-dioxo-2′,3′-dihydrospiro[imidazolidine-4,1′-indene]-1-yl)acetamide;
N-benzyl-N-(1-cyclopropylethyl)-2-(5′-(methylthio)-2,5-dioxo-2′,3′-dihydrospiro[imidazolidine-4,1′-indene]-1-yl)acetamide;
N-benzyl-2-(7′-chloro-2,5-dioxo-2′,3′-dihydrospiro[imidazolidine-4,1′-indene]-1-yl)-N-(1-cyclobutylethyl)acetamide;
N-benzyl-N-(1-cyclopropylethyl)-2-(5′-(hydroxymethyl)-2,5-dioxo-2′,3′-dihydrospiro[imidazolidine-4,1′-indene]-1-yl)acetamide;
N-benzyl-N-(1-cyclopropylethyl)-2-(5′-((difluoromethoxy)methyl)-2,5-dioxo-2′,3′-dihydrospiro[imidazolidine-4,1′-indene]-1-yl)acetamide;
N-benzyl-2-(5′-cyclopropyl-2,5-dioxo-2′,3′-dihydrospiro[imidazolidine-4,1′-indene]-1-yl)-N-(1-cyclopropylethyl)acetamide;
N-benzyl-N-(1-cyclobutylethyl)-2-(3′-methyl-2,5-dioxo-2′,3′-dihydrospiro[imidazolidine-4,1′-indene]-1-yl)acetamide;
N-benzyl-N-(1-cyclobutylethyl)-2-(5′-(difluoromethoxy)-2,5-dioxo-2′,3′-dihydrospiro[imidazolidine-4,1′-indene]-1-yl)acetamide;
N-benzyl-N-(1-cyclopropylethyl)-2-(4′-methyl-2,5-dioxo-2′,3′-dihydrospiro[imidazolidine-4,1′-indene]-1-yl)acetamide;
2-(5′-acetamido-2,5-dioxo-2′,3′-dihydrospiro[imidazolidine-4,1′-indene]-1-yl)-N-benzyl-N-(1-cyclopropylethyl)acetamide;
N-benzyl-N-(1-cyclopropylethyl)-2-(5′-methyl-2,5-dioxo-2′,3′-dihydrospiro[imidazolidine-4,1′-indene]-1-yl)acetamide;
1-(2-(benzyl((S)-1-cyclopropylethyl)amino)-2-oxoethyl)-N-(cyanomethyl)-2,5-dioxo-2′,3′-dihydrospiro[imidazolidine-4,1′-indene]-5′-carboxamide;
N-benzyl-N-(1-cyclopropylethyl)-2-(5′-ethynyl-2,5-dioxo-2′,3′-dihydrospiro[imidazolidine-4,1′-indene]-1-yl)acetamide;
N-benzyl-N-(1-cyclopropylethyl)-2-(5′-(3-hydroxyprop-1-ynyl)-2,5-dioxo-2′,3′-dihydrospiro[imidazolidine-4,1′-indene]-1-yl)acetamide;
N-benzyl-N-(1-cyclobutylethyl)-2-(6′-methoxy-2,5-dioxo-2′,3′-dihydrospiro[imidazolidine-4,1′-indene]-1-yl)acetamide;
N-benzyl-N-(dicyclopropylmethyl)-2-(2,5-dioxo-2′,3′-dihydrospiro[imidazolidine-4,1′-indene]-1-yl)acetamide;
N-benzyl-2-(6′-chloro-2,5-dioxo-2′,3′-dihydrospiro[imidazolidine-4,1′-indene]-1-yl)-N-(1-cyclobutylethyl)acetamide;
N-benzyl-N-(1-cyclopropylethyl)-2-(2,5-dioxo-5′-(1H-pyrazol-5-yl)-2′,3′-dihydrospiro[imidazolidine-4,1′-indene]-1-yl)acetamide;
N-(4-chlorobenzyl)-N—((S)-1-cyclobutylethyl)-2-((S)-2,5-dioxo-2′,3′-dihydrospiro[imidazolidine-4,1′-indene]-1-yl)acetamide;
N-benzyl-N-(1-cyclopropylethyl)-2-(2,5-dioxo-5′-(1H-1,2,3-triazol-4-yl)-2′,3′-dihydrospiro[imidazolidine-4,1′-indene]-1-yl)acetamide;
N-benzyl-N—((S)-1-cyclobutylethyl)-2-((3′R)-3′-hydroxy-2,5-dioxo-2′,3′-dihydrospiro[imidazolidine-4,1′-indene]-1-yl)acetamide;
N-benzyl-N—((S)-1-cyclobutylethyl)-2-((3′S)-3′-hydroxy-2,5-dioxo-2′,3′-dihydrospiro[imidazolidine-4,1′-indene]-1-yl)acetamide;
N-(4-bromobenzyl)-N—((S)-1-cyclobutylethyl)-2-((S)-2,5-dioxo-2′,3′-dihydrospiro[imidazolidine-4,1′-indene]-1-yl)acetamide;
2-(5′-(1H-imidazol-2-yl)-2,5-dioxo-2′,3′-dihydrospiro[imidazolidine-4,1′-indene]-1-yl)-N-benzyl-N-(1-cyclopropylethyl)acetamide;
N—((S)-1-cyclobutylethyl)-2-((S)-2,5-dioxo-2′,3′-dihydrospiro[imidazolidine-4,1′-indene]-1-yl)-N-(4-(methylthio)benzyl)acetamide;
N-benzyl-N-(1-cyclopropylethyl)-2-(2,5-dioxo-5′-(1H-1,2,3-triazol-1-yl)-2′,3′-dihydrospiro[imidazolidine-4,1′-indene]-1-yl)acetamide;
N-benzyl-N-(1-cyclopropylethyl)-2-(2,5-dioxo-5′-(2H-1,2,3-triazol-2-yl)-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(4-fluorobenzyl)acetamide;
N-(4-cyanobenzyl)-N—((S)-1-cyclobutylethyl)-2-((S)-2,5-dioxo-2′,3′-dihydrospiro[imidazolidine-4,1′-indene]-1-yl)acetamide;
4-((N—((S)-1-cyclobutylethyl)-2-((S)-2,5-dioxo-2′,3′-dihydrospiro[imidazolidine-4,1′-indene]-1-yl)acetamido)methyl)benzamide;
N-benzyl-N-(1-cyclopropylethyl)-2-(5′-(methoxymethyl)-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yd)-N-(4-methylbenzyl)acetamide;
N—((S)-1-cyclobutylethyl)-2-((S)-2,5-dioxo-2′,3′-dihydrospiro[imidazolidine-4,1′-indene]-1-yl)-N-(4-methoxybenzyl)acetamide;
N—((S)-1-cyclobutylethyl)-2-((S)-2,5-dioxo-2′,3′-dihydrospiro[imidazolidine-4,1′-indene]-1-yl)-N-(3-methylbenzyl)acetamide;
N—((S)-1-cyclobutylethyl)-2-((S)-2,5-dioxo-2′,3′-dihydrospiro[imidazolidine-4,1′-indene]-1-yl)-N-(2-methylbenzyl)acetamide;
N-((1H-1,2,3-triazol-4-yl)methyl)-N—((S)-1-cyclobutylethyl)-2-((S)-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(4-(methylsulfonyl)benzyl)acetamide;
N-benzyl-N—((S)-1-cyclopropylethyl)-2-(2,3′,5-trioxo-2′,3′-dihydrospiro[imidazolidine-4,1′-indene]-1-yl)acetamide;
N-benzyl-2-(5′-(2-cyanoacetamido)-2,5-dioxo-2′,3′-dihydrospiro[imidazolidine-4,1′-indene]-1-yl)-N—((S)-1-cyclopropylethyl)acetamide;
methyl 1-(2-(benzyl((S)-1-cyclopropylethyl)amino)-2-oxoethyl)-2,5-dioxo-2′,3′-dihydrospiro[imidazolidine-4,1′-indene]-5′-ylcarbamate;
N-benzyl-N—((S)-1-cyclopropylethyl)-2-(7′-fluoro-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(1-phenylethyl)acetamide;
N-benzyl-2-(7′-cyano-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(5′-(3-methylureido)-2,5-dioxo-2′,3′-dihydrospiro[imidazolidine-4,1′-indene]-1-yl)acetamide;
N-benzyl-N—((S)-1-cyclopropylethyl)-2-(5′-(3-methyl-3-(methylcarbamoyl)ureido)-2,5-dioxo-2′,3′-dihydrospiro[imidazolidine-4,1′-indene]-1-yl)acetamide;
N—((S)-1-cyclopropylethyl)-2-((S)-2,5-dioxo-2′,3′-dihydrospiro[imidazolidine-4,1′-indene]-1-yl)-N-(4-ethynylbenzyl)acetamide;
N—((S)-3-cyclobutylethyl)-2-((S)-2,5-dioxo-2′,3′-dihydrospiro[imidazolidine-4,1′-indene]-1-yl)-N-((1-methyl-1H-1,2,3-triazol-4-yl)methyl)acetamide;
N-((1-(cyanomethyl)-1H-1,2,3-triazol-4-yl)methyl)-N—((S)-1-cyclobutylethyl)-2-((S)-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(4-ureidobenzyl)acetamide;
N-benzyl-N—((S)-1-cyclopropylethyl)-2-(5′-(3-hydroxy-3-methylbut-1-ynyl)-2,5-dioxo-2′,3′-dihydrospiro[imidazolidine-4,1′-indene]-1-yl)acetamide;
N-((1-(2-amino-2-oxoethyl)-1H-1,2,3-triazol-4-yl)methyl)-N—((S)-1-cyclobutylethyl)-2-((S)-2,5-dioxo-2′,3′-dihydrospiro[imidazolidine-4,1′-indene]-1-yl)acetamide;
(S)—N-benzyl-N-(1-cyclopropylethyl)-2-(2,5-dioxo-5′-(6-oxo-1,6-dihydropyridin-3-yl)-2′,3′-dihydrospiro[imidazolidine-4,1′-indene]-1-yl)acetamide;
N-benzyl-N—((S)-1-cyclopropylethyl)-2-((S)-2,5-dioxo-5′-(1H-pyrazol-3-yl)-2′,3′-dihydrospiro[imidazolidine-4,1′-indene]-1-yl)acetamide;
N-benzyl-N—((S)-1-cyclopropylethyl)-2-(5′-(methylsulfonamido)-2,5-dioxo-2′,3′-dihydrospiro[imidazolidine-4,1′-indene]-1-yl)acetamide;
N-benzyl-2-(5′-(2-cyanoacetamido)-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(2,5-dioxo-5′-(2H-tetrazol-5-yl)-2′,3′-dihydrospiro[imidazolidine-4,1′-indene]-1-yl)acetamide;
(S)—N-benzyl-N-(1-cyclopropylethyl)-2-(3′-(hydroxyimino)-2,5-dioxo-2′,3′-dihydrospiro[imidazolidine-4,1′-indene]-1-yl)acetamide;
(S)—N-benzyl-N-(1-cyclopropylethyl)-2-(3′-(methoxyamino)-2,5-dioxo-2′,3′-dihydrospiro[imidazolidine-4,1′-indene]-1-yl)acetamide;
N-benzyl-N—((S)-1-cyclopropylethyl)-2-((S)-2,5-dioxo-5′-(6-oxo-1,6-dihydropyridin-3-yl)-2′,3′-dihydrospiro[imidazolidine-4,1′-indene]-1-yl)acetamide;
N-benzyl-N—((S)-1-cyclopropylethyl)-2-((S)-5′-(3-hydroxyprop-1-ynyl)-2,5-dioxo-2′,3′-dihydrospiro[imidazolidine-4,1′-indene]-1-yl)acetamide;
N-benzyl-N—((S)-1-cyclopropylethyl)-2-((S)-2,5-dioxo-5′-(2H-1,2,3-triazol-4-yl)-2′,3′-dihydrospiro[imidazolidine-4,1′-indene]-1-yl)acetamide;
N-benzyl-N—((S)-1-cyclopropylethyl)-2-(5′-(3-hydroxypyrrolidin-1-yl)-2,5-dioxo-2′,3′-dihydrospiro[imidazolidine-4,1′-indene]-1-yl)acetamide;
N-benzyl-N—((S)-1-cyclopropylethyl)-2-((S)-5′-(3-hydroxy-3-methylbut-1-ynyl)-2,5-dioxo-2′,3′-dihydrospiro[imidazolidine-4,1′-indene]-1-yl)acetamide;
N-benzyl-2-((S)-5′-cyano-2,5-dioxo-2′,3′-dihydrospiro[imidazolidine-4,1′-indene]-1-yl)-N—((S)-1-cyclopropylethyl)acetamide;
1-(2-(benzyl((S)-1-cyclopropylethyl)amino)-2-oxoethyl)-2,5-dioxo-2′,3′-dihydrospiro[imidazolidine-4,1′-indene]-3′-carboxylic acid;
N-benzyl-N—((S)-1-cyclopropylethyl)-2-((S)-5′-(3-methylureido)-2,5-dioxo-2′,3′-dihydrospiro[imidazolidine-4,1′-indene]-1-yl)acetamide;
N-benzyl-N—((S)-1-cyclopropylethyl)-2-(5′-(3-hydroxyazetidin-1-yl)-2,5-dioxo-2′,3′-dihydrospiro[imidazolidine-4,1′-indene]-1-yl)acetamide;
N-benzyl-2-((S)-5′-(2′-cyanoacetamido)-2,5-dioxo-2′,3′-dihydrospiro[imidazolidine-4, 1′-indene]-1-yl)-N—((S)-1-cyclopropylethyl)acetamide;
1-(2-(benzyl((S)-1-cyclopropylethyl)amino)-2-oxoethyl)-2,5-dioxo-2′,3′-dihydrospiro[imidazolidine-4,1′-indene]-3′-carboxamide;
N-benzyl-2-(3′-cyano-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(5′-(2,4-dioxo-3,4-dihydropyrimidin-1(2H)-yl)-2,5-dioxo-2′,3′-dihydrospiro[imidazolidine-4,1′-indene]-1-yl)acetamide;
N-(1-(2-(benzyl((S)-1-cyclopropylethyl)amino)-2-oxoethyl)-2,5-dioxo-2′,3′-dihydrospiro[imidazolidine-4,1′-indene]-5′-ylacrylamide;
N-benzyl-2-(5′-(2-cyano-1-hydroxyallyl)-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(5′-(2,4-dioxo-1,2,3,4-tetrahydropyrimidin-5-yl)-2,5-dioxo-2′,3′-dihydrospiro[imidazolidine-4,1′-indene]-1-yl)acetamide;
1-(2-(benzyl((S)-1-cyclopropylethyl)amino)-2-oxoethyl)-2,5-dioxo-2′,3′-dihydrospiro[imidazolidine-4,1′-indene]-5′-carboxylic acid;
(S)—N-(1-(2-(benzyl(1-cyclopropylethyl)amino)-2-oxoethyl)-2,5-dioxo-2′,3′-dihydrospiro[imidazolidine-4,1′-indene]-5′-yl)-3-oxobutanamide;
N-(1-(2-(benzyl((S)-1-cyclopropylethyl)amino)-2-oxoethyl)-2,5-dioxo-2′,3′-dihydrospiro[imidazolidine-4,1′-indene]-5′-yl)-2-cyano-3-oxobutanamide;
(E)-N-(1-(2-(benzyl((S)-1-cyclopropylethyl)amino)-2-oxoethyl)-2,5-dioxo-2′,3′-dihydrospiro[imidazolidine-4,1′-indene]-5′-yl)-4-(piperidin-1-yl)but-2-enamide;
N-benzyl-2-((S)-2,5-dioxo-2′,3′-dihydrospiro[imidazolidine-4,1′-indene]-1-yl)-N-(1-(oxetan-3-yl)ethyl)acetamide;
2-amino-N-((1′S)-1-((4S)-3-benzyl-4-cyclopropyl-2-oxopentyl)-2,5-dioxo-2′,3′-dihydrospiro[imidazolidine-4,1′-indene]-5′-yl)-2-methylpropan amide;
(S)-2-amino-N—((S)-1-(2-(benzyl((S)-1-cyclopropylethyl)amino)-2-oxoethyl)-2,5-dioxo-2′,3′-dihydrospiro[imidazolidine-4,1′-indene]-5′-ylpropanamide;
N-benzyl-N—((S)-1-cyclopropylethyl)-2-(5′-(2-hydroxy-5-oxo-2,5-dihydrofuran-2-yl)-2,5-dioxo-2′,3′-dihydrospiro[imidazolidine-4,1′-indene]-1-yl)acetamide;
5′-(azetidin-3-ylmethylamino)-1-((4S)-3-benzyl-4-cyclopropyl-2-oxopentyl)-2′,3′-dihydrospiro[imidazolidine-4,1′-indene]-2,5-dione;
1-amino-N—((S)-1-(2-(benzyl((S)-1-cyclopropylethyl)amino)-2-oxoethyl)-2,5-dioxo-2′,3′-dihydrospiro[imidazolidine-4,1′-indene]-5′-yl)cyclobutanecarboxamide;
N-benzyl-N—((S)-1-cyclopropylethyl)-2-(2,5-dioxo-5′-(thiazol-2-ylamino)-2′,3′-dihydrospiro[imidazolidine-4,1′-indene]-1-yl)acetamide;
(S)-2-(5′-(azetidin-3-ylamino)-2,5-dioxo-2′,3′-dihydrospiro[imidazolidine-4,1′-indene]-1-yl)-N-benzyl-N-(1-cyclopropylethyl)acetamide;
N-benzyl-N-(1-cyclopropylethyl)-2-(2,5-dioxo-1′,3′-dihydrospiro[imidazolidine-4,2′-indene]-1-yl)acetamide;
N-benzyl-N-(1-cyclopropylethyl)-2-(8′-methoxy-2,5-dioxo-3′,4′-dihydro-1′H-spiro[imidazolidine-4,2′-naphthalen]-1-yl)acetamide;
N-benzyl-N-(1-cyclobutylethyl)-2-(3′,3′-dimethyl-2,5-dioxo-2′,3′-dihydrospiro[imidazolidine-4,1′-indene]-1-yl)acetamide;
N-benzyl-2-[5′-(5-cyano-6-oxo-1,6-dihydropyridin-3-yl)-2,5-dioxo-2′,3′-dihydrospiro[imidazolidine-4,1′-indene]-1-yl]-N-(1-cyclopropylethyl)acetamide;
(2R)-2-amino-N-[(4S)-1-({benzyl[(1S)-1-cyclopropylethyl]carbamoyl}methyl)-2,5-dioxo-2′,3′-dihydrospiro[imidazolidine-4,1′-indene]-5′-yl]propanamide;
N-benzyl-N-[(1S)-1-cyclopropylethyl]-2-{5′-[(1H-imidazol-2-yl)amino]-2,5-dioxo-2′,3′-dihydro spiro[imidazolidine-4,1′-indene]-1-yl}acetamide;
1-amino-N-[(4S)-1-({benzyl[(1S)-1-cyclopropylethyl]carbamoyl}methyl)-2,5-dioxo-2′,3′-dihydrospiro[imidazolidine-4,1′-indene]-5′-yl]cyclopropane-1-carboxamide;
N-benzyl-N-[(1R)-1-cyclopropyl-2,2,2-trifluoroethyl]-2-[(4S)-2,5-dioxo-2′,3′-dihydrospiro[imidazolidine-4,1′-indene]-1-yl]acetamide;
N-benzyl-N-[(1S)-1-cyclopropylethyl]-2-{5′-[(oxetan-3-ylmethyl)amino]-2,5-dioxo-2′,3′-dihydrospiro[imidazolidine-4,1′-indene]-1-yl}acetamide;
N-benzyl-N-[(1S)-1-cyclopropylethyl]-2-{1,1,2′,5′-tetraoxo-2H-spiro[1λ⁶-benzothiophene-3,4′-imidazolidine]-1′-yl}acetamide;
2-[5′-(azetidin-3-yloxy)-2,5-dioxo-2′,3′-dihydrospiro[imidazolidine-4,1′-indene]-1-yl]-N-benzyl-N-[(1S)-1-cyclopropylethyl]acetamide;
N-[(2-amino-1,3-thiazol-5-yl)methyl]-N-[(1S)-1-cyclopropylethyl]-2-[(4S)-2,5-dioxo-2′,3′-dihydrospiro[imidazolidine-4,1′-indene]-1-yl]acetamide;
N-[(1S)-1-cyclopropylethyl]-N-{[2-(dimethylamino)-1,3-thiazol-5-yl]methyl}-2-[(4S)-2,5-dioxo-2′,3′-dihydrospiro[imidazolidine-4,1′-indene]-1-yl]acetamide;
N-[(1S)-1-cyclopropylethyl]-N-{[6-(dimethylamino)pyridin-3-yl]methyl}-2-[(4S)-2,5-dioxo-2′,3′-dihydrospiro[imidazolidine-4,1′-indene]-1-yl]acetamide;
N-[(2-amino-1,3-thiazol-4-yl)methyl]-N-[(1S)-1-cyclopropylethyl]-2-[(4S)-2,5-dioxo-2′,3′-dihydrospiro[imidazolidine-4,1′-indene]-1-yl]acetamide;
N-[(1S)-1-cyclopropylethyl]-N-[(4-fluorophenyl)methyl]-2-{1,1,2′,5′-tetraoxo-2H-spiro[1λ⁶-benzothiophene-3,4′-imidazolidine]-1′-yl}acetamide;
N-[(1S)-1-cyclopropylethyl]-2-[(4S)-2,5-dioxo-2′,3′-dihydrospiro[imidazolidine-4,1′-indene]-1-yl]-N-[(6-hydroxypyridin-3-yl)methyl]acetamide;
N-[(1S)-1-cyclopropylethyl]-2-[(4S)-2,5-dioxo-2′,3′-dihydrospiro[imidazolidine-4,1′-indene]-1-yl]-N-[(6-methoxypyridin-3-ylmethyl]acetamide;
N-[(1S)-1-cyclopropylethyl]-N-{[2-(dimethylamino)-1,3-thiazol-4-yl]methyl}-2-[(4S)-2,5-dioxo-2′,3′-dihydrospiro[imidazolidine-4,1′-indene]-1-yl]acetamide;
N-benzyl-N-[(1R)-1-cyclopropyl-2,2,2-trifluoroethyl]-2-{1,1,2′,5′-tetraoxo-2H-spiro[1λ⁶-benzothiophene-3,4′-imidazolidine]-1′-yl}acetamide;
N-[(6-amino-5-methoxypyridin-3-yl)methyl]-N-[(1S)-1-cyclopropylethyl]-2-[(4S)-2,5-dioxo-2′,3′-dihydrospiro[imidazolidine-4,1′-indene]-1-yl]acetamide;
N-[(6-aminopyridin-3-yl)methyl]-N-[(1S)-1-cyclopropylethyl]-2-[(4S)-2,5-dioxo-2′,3′-dihydrospiro[imidazolidine-4,1′-indene]-1-yl]acetamide;
N-benzyl-2-{6-bromo-1,1,2′,5′-tetraoxo-2H-spiro[1λ⁶-benzothiophene-3,4′-imidazolidine]-1′-yl}-N-[(1S)-1-cyclopropylethyl]acetamide;
N-benzyl-N-[(1S)-1-cyclopropylethyl]-2-[1,1,2′,5′-tetraoxo-6-(1H-pyrazol-3-yl)-2H-spiro[1λ⁶-benzothiophene-3; 4′-imidazolidine]-1′-yl]acetamide;
2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide;
2-(6-bromo-1,1-dioxido-2′,5′-dioxo-1′H-spiro[1-benzothiophene-3,4′-imidazolidin]-1′-yl) -N-[(1S)-1-cyclopropylethyl]-N-(4-fluorobenzyl)acetamide;
N-[(1S)-1-cyclopropylethyl]-2-[2,5-dioxo-5′-(6-oxo-1,6-dihydropyridin-3-yl)-1′,3′-dihydro-1H-spiro[imidazolidine-4,2′-inden]-1-yl]-N-(4-fluorobenzyl)acetamide;
2-(5′-amino-2,5-dioxo-1′,3′-dihydro-1H-spiro[imidazolidine-4,2′-inden]-1-yl)-N-[(1S)-1-cyclopropylethyl]-N-(4-fluorobenzyl)acetamide;
N-[(1S)-1-cyclopropylethyl]-2-[1,1-dioxido-2′,5′-dioxo-6-(6-oxo-1,6-dihydropyridin-3-yl)-1′H-spiro[1-benzothiophene-3,4′-imidazolidin]-1′-yl]-N-(4-fluorobenzyl)acetamide;
2-(6-amino-1,1-dioxido-2′,5′-dioxo-1′H-spiro[1-benzothiophene-3,4′-imidazolidin]-1-yl)-N-[(1S)-1-cyclopropylethyl]-N-(4-fluorobenzyl)acetamide;
N-[(1S)-1-cyclopropylethyl]-N-(4-fluorobenzyl)-2-{5′-[(methylcarbamoyl)amino]-2,5-dioxo-1′,3′-dihydro-1H-spiro[imidazolidine-4,2′-inden]-1-yl}acetamide;
2-[5′-(acetylamino)-2,5-dioxo-1′,3′-dihydro-1H-spiro[imidazolidine-4,2′-inden]-1-yl]-N-[(1S)-1-cyclopropylethyl]-N-(4-fluorobenzyl)acetamide;
N-[(1S)-1-cyclopropylethyl]-N-(4-fluorobenzyl)-2-{6-[(methylcarbamoyl)amino]-1,1-dioxido-2′,5′-dioxo-1′H-spiro[1-benzothiophene-3,4′-imidazolidin]-1′-yl}acetamide;
N-(4-fluorobenzyl)-2-(1′-methyl-2,5-dioxo-2′,3′-dihydro-1H,1′H-spiro[imidazolidine-4,4′-quinolin]-1-yl)-N-[(2S)-1,1,1-trifluoropropan-2-yl]acetamide;
N-[(1S)-1-cyclopropylethyl]-N-(4-fluorobenzyl)-2-(3′-hydroxy-2,5-dioxo-2′,3′-dihydro-1H-spiro[imidazolidine-4,1′-inden]-1-yl)acetamide;
N-(4-fluorobenzyl)-N-[(2S)-1,1,1-trifluoropropan-2-yl]-2-(2,3′,5-trioxo-2′,3′-dihydro-1H-spiro[imidazolidine-4,1′-inden]-1-yl)acetamide;
N-[(1S)-1-cyclopropylethyl]-N-(4-fluorobenzyl)-2-(1′-methyl-2,5-dioxo-2′,3′-dihydro-1H,1′H-spiro[imidazolidine-4,4′-quinolin]-1-yl)acetamide
N-(4-fluorobenzyl)-2-[(3′E)-3′-(hydroxyimino)-2,5-dioxo-2′,3′-dihydro-1H-spiro[imidazolidine-4,1′-indene]-1-yl]-N-[(2S)-1,1,1-trifluoropropan-2-yl]acetamide;
N-[(1S)-1-cyclopropylethyl]-N-(4-fluorobenzyl)-2-(2,3′,5-trioxo-2′,3′-dihydro-1H-spiro[imidazolidine-4,1′-inden]-1-yl)acetamide;
N-(4-fluorobenzyl)-2-(3′-hydroxy-2,5-dioxo-2′,3′-dihydro-1H-spiro[imidazolidine-4,1′-inden]-1-yl)-N-[(2S)-1,1,1-trifluoropropan-2-yl]acetamide;
2-[6-(acetylamino)-1,1-dioxido-2′,5′-dioxo-1′H-spiro[1-benzothiophene-3,4′-imidazolidin]-1′-yl]-N-[(1S)-1-cyclopropylethyl]-N-(4-fluorobenzyl)acetamide;
2-(7′-bromo-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
N-(4-fluorobenzyl)-2-(3′-fluoro-2,5-dioxo-2′,3′-dihydro-1H-spiro[imidazolidine-4,1′-inden]-1-yl)-N-[(2S)-1,1,1-trifluoropropan-2-yl]acetamide;
2-(3′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[3′-(methylamino)-2,5-dioxo-2′,3′-dihydro-1H-spiro[imidazolidine-4,1′-inden]-1-yl]-N-[(2S)-1,1,1-trifluoropropan-2-yl]acetamide;
2-(7′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
2-(6-bromo-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide;
N-[(1S)-1-cyclopropylethyl]-N-(4-fluorobenzyl)-2-{7′-[(methylcarbamoyl)amino]-2,5-dioxo-3′,4′-dihydro-1H,1′H-spiro[imidazolidine-4,2′-naphthalen]-1-yl}acetamide;
2-[7′-(acetylamino)-2,5-dioxo-3′,4′-dihydro-1H,1′H-spiro[imidazolidine-4,2′-naphthalen]-1-yl]-N-[(1S)-1-cyclopropylethyl]-N-(4-fluorobenzyl)acetamide;
N-(4-fluorobenzyl)-2-(2′-methyl-2,5-dioxo-2′,3′-dihydro-1H-spiro[imidazolidine-4,1′-inden]-1-yl)-N-[(2S)-1,1,1-trifluoropropan-2-yl]acetamide;
N-[1-(2-{[(1S)-1-cyclopropylethyl](4-fluorobenzyl)amino}-2-oxoethyl)-2,5-dioxo-3′,4′-dihydro-1′H-spiro[imidazolidine-4,2′-naphthalen]-7′-yl]-5-oxo-D-prolinamide;
2-{7′-[1-(2-amino-2-oxoethyl)-1H-pyrazol-4-yl]-2,5-dioxo-3′,4′-dihydro-1H,1′H-spiro[imidazolidine-4,2′-naphthalen]-1-yl}-N-[(1S)-1-cyclopropylethyl]-N-(4-fluorobenzyl)acetamide;
2-(5′-bromo-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-N-[(2S)-1,1,1-trifluoropropan-2-yl]-2-(2,2′,5-trioxo-2′,3′-dihydro-1H,1′H-spiro[imidazolidine-4,4′-quinolin]-1-yl)acetamide;
2-(2; 5-dioxo-1H-spiro[imidazolidine-4,1′-inden]-1-yl)-N-(4-fluorobenzyl)-N-[(2S)-1,1,1-trifluoropropan-2-yl]acetamide;
2-{5′-[1-(2-amino-2-oxoethyl)-1H-pyrazol-4-yl]-2,3′,5-trioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-N-[(2S)-1,1,1-trifluoropropan-2-yl]-2-[2,3′,5-trioxo-5′-(6-oxo-1,6-dihydropyridin-3-yl)-2′,3′-dihydro-1H-spiro[imidazolidine-4,1′-inden]-1-yl]acetamide;
N-[(1S)-1-cyclopropylethyl]-2-[(4R)-2,5-dioxo-2′,3′-dihydro-1H-spiro[imidazoline-4,1′-inden]-1-yl]-N-(4-fluorobenzyl)acetamide;
N-[(1S)-1-cyclopropylethyl]-2-[(4S)-2,5-dioxo-2′,3′-dihydro-1H-spiro[imidazolidine-4,1′-inden]-1-yl]-N-(4-fluorobenzyl)acetamide;
2-(7′-bromo-1′-methyl-2,5-dioxo-2′,3′-dihydro-1H,1′H-spiro[imidazolidine-4,4′-quinolin]-1-yl)-N-[(1S)-1-cyclopropylethyl]-N-(4-fluorobenzyl)acetamide;
2-[5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-(4′-hydroxy-2,5-dioxo-3′,4′-dihydro-1H,2′H-spiro[imidazolidine-4,1′-naphthalen]-1-yl)-N-[(2S)-1,1,1-trifluoropropan-2-yl]acetamide;
2-(6-{1-[2-(dimethylamino)-2-oxoethyl]-1H-pyrazol-4-yl}-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide;
2-[5′-(acetylamino)-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[5′-(acetylamino)-2,5-dioxo-2′,3′-dihydro-1H-spiro[imidazolidine-4,1′-inden]-1-yl]-N-[(1S)-1-cyclopropylethyl]-N-(4-fluorobenzyl)acetamide;
N-(4-fluorobenzyl)-2-(5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-(3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
2-(6′-bromo-2,5-dioxo-3′,4′-dihydro-1H, 1′H-spiro[imidazolidine-4,2′-naphthalen]-1-yl)-N-[(1S)-1-cyclopropylethyl]-N-(4-fluorobenzyl)acetamide;
2-(6′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
2-(7′-amino-1′-methyl-2,5-dioxo-2′,3′-dihydro-1H,1′H-spiro[imidazolidine-4,4′-quinolin]-1-yl)-N-[(1S)-1-cyclopropylethyl]-N-(4-fluorobenzyl)acetamide;
N-[(1S)-1-cyclopropylethyl]-N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}acetamide;
N-[(1S)-1-cyclopropylethyl]-N-(4-fluorobenzyl)-2-{6′-[(methylcarbamoyl)amino]-2,5-dioxo-3′,4′-dihydro-1H,1H-spiro[imidazolidine-4,2′-naphthalen]-1-yl}acetamide;
2-[6′-(acetylamino)-2,5-dioxo-3′,4′-dihydro-1H,1′H-spiro[imidazolidine-4,2′-naphthalen]-1-yl]-N-[(1S)-1-cyclopropylethyl]-N-(4-fluorobenzyl)acetamide;
2-(6-amino-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide;
N-(4-fluorobenzyl)-2-(6-{1-[2-(methylamino)-2-oxoethyl]-1H-pyrazol-4-yl}-1,1-dioxido-2′,5′-dioxo-1′H-spiro[1-benzothiophene-3,4′-imidazolidin]-1′-yl)-N-[(2S)-1,1,1-trifluoropropan-2-yl]acetamide;
tert-butyl {4-[1-(2-{(4-fluorobenzyl)[(2S)-1,1,1-trifluoropropan-2-yl]amino}-2-oxoethyl)-2,3′,5-trioxo-2′,3′-dihydrospiro[imidazolidine-4,1′-inden]-5′-yl]-1H-pyrazol-1-yl}acetate;
N-(4-fluorobenzyl)-2-{3′-fluoro-5′-[1-(2-methylpropyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
{4-[1-(2-{(4-fluorobenzyl)[(2S)-1,1,1-trifluoropropan-2-yl]amino}-2-oxoethyl)-2,3′,5-trioxo-2′,3′-dihydrospiro[imidazolidine-4,1′-inden]-5′-yl]-1H-pyrazol-1-yl}acetic acid;
2-(5′-{1-[2-(dimethylamino)-2-oxoethyl]-1H-pyrazol-4-yl}-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-(5′-{1-[2-(3-hydroxyazetidin-1-yl)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-{6-[(methylcarbamoyl)amino]-1,1-dioxido-2′,5′-dioxo-1′H-spiro[1-benzothiophene-3,4′-imidazolidin]-1′-yl}-N-[(2S)-1,1,1-trifluoropropan-2-yl]acetamide;
2-(5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-{5′-[1-(2-amino-2-oxoethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-yl]acetamide;
2-{5′-[1-(2-amino-2-oxoethyl)-1H-pyrazol-4-yl]-3′-hydroxy-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-[(4S)-5′-{1-[2-(dimethylamino)-2-oxoethyl]-1H-pyrazol-4-yl}-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4S)-5′-{1-[2-(dimethylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-N-[(2S)-1,1,1-trifluoropropan-2-yl]-2-[(4R)-2,3′,5-trioxo-2′,3′-dihydro-1H-spiro[imidazolidine-4,1′-inden]-1-yl]acetamide;
N-(4-fluorobenzyl)-N-[(2S)-1,1,1-trifluoropropan-2-yl]-2-[(4S)-2,3′,5-trioxo-2′,3′-dihydro-1H-spiro[imidazolidine-4,1′-inden]-1-yl]acetamide;
N-(4-fluorobenzyl)-2-[5′-(1-methyl-1H-pyrazol-4-yl)-2,3′,5-trioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(7′-bromo-1′-methyl-2,5-dioxo-2′,3′-dihydro-1H,1′H-spiro[imidazolidine-4,4′-quinolin]-1-yl)-N-[(1S)-1-cyclopropyl-2,2,2-trifluoroethyl]-N-(4-fluorobenzyl)acetamide;
N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}-N-[(2S)-1,1,1-trifluoropropan-2-yl]acetamide;
N-(4-fluorobenzyl)-2-[(3′R,4R)-3′-hydroxy-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(3′S,4R)-3′-fluoro-2,5-dioxo-2′,3′-dihydro-1H-spiro[imidazolidine-4,1′-inden]-1-yl]-N-[(2S)-1,1,1-trifluoropropan-2-yl]acetamide;
2-(7′-bromo-2,2′,5-trioxo-2′,3′-dihydro-1H,1′H-spiro[imidazolidine-4,4′-quinolin]-1-yl)-N-(4-fluorobenzyl)-N-[(2S)-1,1,1-trifluoropropan-2-yl]acetamide;
N-(4-fluorobenzyl)-2-(7′-{1-[2-(methylamino)-2-oxoethyl]-1H-pyrazol-4-yl}-2,2′,5-trioxo-2′,3′-dihydro-1H,1′H-spiro[imidazolidine-4,4′-quinolin]-1-yl)-N-[(2S)-1,1,1-trifluoropropan-2-yl]acetamide;
2-{7′-[1-(2-amino-2-oxoethyl)-1H-pyrazol-4-yl]-2,2′,5-trioxo-2′,3′-dihydro-1H,1′H-spiro[imidazolidine-4,4′-quinolin]-1-yl}-N-(4-fluorobenzyl)-N-[(2S)-1,1,1-trifluoropropan-2-yl]acetamide;
N-[(1S)-1-cyclopropyl-2,2,2-trifluoroethyl]-N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}acetamide;
2-(5′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2,2,2-trifluoro-N-[1-(2-{(4-fluorobenzyl)[(2S)-1,1,1-trifluoropropan-2-yl]amino}-2-oxoethyl)-1′-methyl-2,5-dioxo-2′,3′-dihydro-1′H-spiro[imidazolidine-4,4′-quinolin]-7′-yl]acetamide;
N-[1-(2-{[(1S)-1-cyclopropylethyl](4-fluorobenzyl)amino}-2-oxoethyl)-1′-methyl-2,5-dioxo-2′,3′-dihydro-1′H-spiro[imidazolidine-4,4′-quinolin]-7′-yl]-2,2,2-trifluoroacetamide;
N-(4-fluorobenzyl)-2-[(3′S,4S)-3′-hydroxy-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[3′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(5′-bromo-3′-hydroxy-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[(3′R,4S)-3′-fluoro-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-{5′-[(methylcarbamoyl)amino]-2,3′,5-trioxo-2′,3′-dihydro-1H-spiro[imidazolidine-4,1′-inden]-1-yl}-N-[(2S)-1,1,1-trifluoropropan-2-yl]acetamide;
2-(5′-amino-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide;
2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{3′-hydroxy-5′-[1-(2-methyl)propyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(4S)-5′-bromo-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-(5′-bromo-3′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-{(4S)-5′-[1-(2-amino-2-oxoethyl)-1H-pyrazol-4-yl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-[(1S)-1-cyclopropylethyl]-N-(4-fluorobenzyl)-2-[1′-methyl-7′-(1-methyl-1H-pyrazol-4-yl)-2,5-dioxo-2′,3′-dihydro-1H,1′H-spiro[imidazolidine-4,4′-quinolin]-1-yl]acetamide;
N-(4-fluorobenzyl)-2-[7′-(1-methyl-1H-pyrazol-4-yl)-2,2′,5-trioxo-2′,3′-dihydro-1H,1′H-spiro[imidazolidine-4,4′-quinolin]-1-yl]-N-[(2S)-1,1,1-trifluoropropan-2-yl]acetamide;
N-(4-fluorobenzyl)-2-[(4S)-5′-(1-methyl-1H-pyrazol-4-yl)-2,3′,5-trioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(3′R,4S)-3′-hydroxy-2,5-dioxo-2′,3′-dihydro-1H-spiro[imidazolidin-4,1′-inden]-1-yl]-N-[(2S)-1,1,1-trifluoropropan-2-yl]acetamide;
N-(4-fluorobenzyl)-2-[(3′S,4S)-3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(4S)-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(3′S,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(4S)-5′-{1-[2-(methylamino)-2-oxoethyl]-1H-pyrazol-4-yl]}-2,3′,5-trioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(3′S,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(3′R,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(3′S,4S)-5′-bromo-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(3′R,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(3′R,4S)-5′-bromo-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{(4S)-5′-[1-(2-amino-2-oxoethyl)-1H-pyrazol-4-yl]-2,3′,5-trioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-{(3′S,4S)-5′-[1-(2-amino-2-oxoethyl)-1H-pyrazol-4-yl]-3′-hydroxy-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-[(3′S,4S)-5′-{1-[2-(dimethylamino)-2-oxoethyl]-1H-pyrazol-4-yl}-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3′R,4S)-5′-{1-[2-(dimethylamino)-2-oxoethyl]-1H-pyrazol-4-yl}-3′-fluoro-2,5-dioxo-2′,3′-dihydro-1H-spiro[imidazolidine-4,1-inden]-1-yl]-N-(4-fluorobenzyl)-N-[(2S)-1,1,1-trifluoropropan-2-yl]acetamide;
N-(4-fluorobenzyl)-2-[(4S)-5′-{1-[2-(methylamino)-2-oxoethyl]-1H-pyrazol-4-yl}-2,5-dioxo-1H-spiro[imidazolidine-4,1′-inden]-1-yl]-N-[(2S)-1,1,1-trifluoropropan-2-yl]acetamide;
N-(3,4-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(2,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1′-trifluoropropan-2-yl]acetamide;
N-[(1S)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-[(1S)-1-cyclopropyl-2,2,2-trifluoroethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-[(1R)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-benzyl-N-[(1S)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-benzyl-N-[(1R)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide;
2-(4′-bromo-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[4′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-[(3′S,4S)-5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3S)-6-amino-1,1-dioxido-2′,5′-dioxo-1H-spiro[1-benzothiophene-3,4′-imidazolidin]-1′-yl]-N-(4-fluorobenzyl)-N-[(2S)-1,1,1-trifluoropropan-2-yl]acetamide;
2-[(3′R,4S)-5′-bromo-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-(5′-bromo-6′-fluoro-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-(5′-bromo-6′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-(6′-fluoro-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-(6′-fluoro-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-[4′-(1-methyl-1,2,3,6-tetrahydropyridin-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[(3′R,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(3R)-6-amino-1,1-dioxido-2′,5′-dioxo-1′H-spiro[1-benzothiophene-3,4′-imidazolidin]-1′-yl]-N-(4-fluorobenzyl)-N-[(2S)-1,1,1-trifluoropropan-2-yl]acetamide;
2-{2,5-dioxo-5′-[(2,2,2-trifluoroethyl)amino]-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-[(4S)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4R)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{(3′R,4S)-5′-[1-(2-amino-2-oxoethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-(5′-bromo-6′-fluoro-3′-hydroxy-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-(6′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
2-{5′-[1-(2-amino-2-oxoethyl)-1H-pyrazol-4-yl]-6′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-{6′-fluoro-5′-[1-(2-hydroxyethyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-[6′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(5′-bromo-3′,6′-difluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
tert-butyl 4-[1-(2-{(4-fluorobenzyl)[(2S)-1,1,1-trifluoropropan-2-yl]amino}-2-oxoethyl)-2,5-dioxo-2′,3′-dihydrospiro[imidazolidine-4,1′-inden]-4′-yl]-3,6-dihydropyridine-1(2H)-carboxylate;
2-[2,5-dioxo-4′-(1,2,3,6-tetrahydropyridin-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{5′-[1-(2-amino-2-oxoethyl)-1H-pyrazol-4-yl]-3′,6′-difluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-{4-[3′,6′-difluoro-1-(2-{(4-fluorobenzyl) [(2S)-1,1,1-trifluoropropan-2-yl]amino}-2-oxoethyl)-2,5-dioxo-2′,3′-dihydro-1H-spiro[imidazolidine-4,1′-inden]-5′-yl]-1H-pyrazol-1-yl}-N,N-dimethylacetamide;
2-[3′,6′-difluoro-5′-(1-methyl-1H-pyrazol-4-yl)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{3′,6′-difluoro-5′-[1-(2-hydroxyethyl)-H-pyrazol-4-yl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-[(4R)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-[(4S)-2,5-dioxo-5′-(1H-pyrazol-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{5′-[(1-methyl-1H-pyrazol-3-yl)amino]-2,5-dioxo-2′,3′-dihydro-1H-spiro[imidazolidine-4,1′-inden]-1-yl}-N-[(2S)-1,1,1-trifluoropropan-2-yl]acetamide;
2-[(4S)-5′-amino-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{4′-[(dimethylamino)methyl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-[4′-(morpholin-4-ylmethyl)-2,5-dioxo-2′,3′-dihydro-1H-spiro[imidazolidine-4,1′-inden]-1-yl]-N-[(2S)-1,1,1-trifluoropropan-2-yl]acetamide;
2-[2,5-dioxo-4′-(pyrrolidin-1-ylmethyl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-(4′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
methyl trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylate;
trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylic acid;
trans-4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]cyclohexanecarboxylic acid;
2-[(4R)-7′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
trans-4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}cyclohexanecarboxylic acid;
2-[(4S)-7′-bromo-2,5-dioxo-2′,3′-dihydro-H-spiro[imidazolidinne-4,1′-inden]-1-yl]-N-(4-fluorobenzyl)-N-[(2S)-1,1,1-trifluoropropan-2-yl]acetamide;
ethyl ({4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]piperidin-1-yl}sulfonyl)carbamate;
2-[(4S)-5′-{[(2-cyanoethyl)carbamoyl]amino}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{(4S)-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(4S)-5′-[(methylcarbamoyl)amino]-2,3′,5-trioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(3′S,4S)-3′-hydroxy-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(3′R,4S)-3′-fluoro-5′-[1-(2-hydroxyethyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
2-[4′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4R)-5′-bromo-6′-fluoro-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{(3′R,4S)-3′-fluoro-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{7′-[(methylcarbamoyl)amino]-2,2′,5-trioxo-2′,3′-dihydro-1H, 1′H-spiro[imidazolidine-4,4′-quinolin]-1-yl}-N-[(2S)-1,1,1-trifluoropropan-2-yl]acetamide;
tert-butyl (4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetate;
(4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetic acid;
{4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
(4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}cyclohexylidene)acetic acid;
N-(4-fluorobenzyl)-2-[(3′R,4S)-3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-[(4R)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(3′E,4S)-3′-(hydroxyimino)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-[(3′R,4S)-3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{4-[(3′R,4S)-3′,6′-difluoro-1-(2-{(4-fluorobenzyl)[(2S)-1,1,1-trifluoropropan-2-yl]amino}-2-oxoethyl)-2,5-dioxo-2′,3′-dihydro-H-spiro[imidazolidine-4,1′-inden]-5′-yl]-1H-pyrazol-1-yl}-N,N-dimethylacetamide;
2-[(3′S,4R)-3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{4-[(3′S,4R)-3′,6′-difluoro-1-(2-{(4-fluorobenzyl)[(2S)-1,1,1-trifluoropropan-2-yl]amino}-2-oxoethyl)-2,5-dioxo-2′,3′-dihydro-H-spiro[imidazolidine-4,1′-inden]-5′-yl]-1H-pyrazol-1-yl}-N,N-dimethylacetamide;
ethyl [(4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}piperidin-1-yl)sulfonyl]carbamate;
tert-butyl 4-[{[(4 S)-5′-bromo-2,5-dioxo-2′,3′-dihydro-1H-spiro[imidazolidine-4,1′-inden]-1-yl]acetyl}(4-fluorobenzyl)amino]piperidine-1-carboxylate;
ethyl ({4-[{[(4S)-5′-amino-2,5-dioxo-2′,3′-dihydro-1H-spiro[imidazolidine-4,1′-inden]-1-yl]acetyl}(4-fluorobenzyl)amino]piperidin-1-yl}sulfonyl)carbamate;
ethyl ({3,3-difluoro-4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]pyrrolidin-1-yl}sulfonyl)carbamate;
ethyl ({3,3-difluoro-4-[(4-fluorobenzyl) {[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]acetyl}amino]pyrrolidin-1-yl}sulfonyl)carbamate;
2-{(3′R,4S)-5′-[1-(difluoromethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-[2,5-dioxo-4′-(piperidin-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(4S)-7′-hydroxy-2,5-dioxo-2′,3′-dihydro-1H-spiro[imidazolidine-4,1′-inden]-1-yl]-N-[(2S)-1,1,1-trifluoropropan-2-yl]acetamide;
tert-butyl {4-[{[(4S)-5′-bromo-2,5-dioxo-2′,3′-dihydro-1H-spiro[imidazolidine-4,1′-inden]-1-yl]acetyl}(4-fluorobenzyl)amino]cyclohexylidene}acetate;
tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate;
{4-[{[(4S)-5′-amino-2,5-dioxo-2′,3′-dihydro-1H-spiro[imidazolidine-4,1′-inden]-1-yl]acetyl}(4-fluorobenzyl)amino]cyclohexylidene}acetic acid;
2-[(4S)-5′-bromo-6′-fluoro-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3′R,4R)-5′-bromo-6′-fluoro-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3′S,4S)-5′-bromo-6′-fluoro-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]-N-(tetrahydro-2H-pyran-4-yl)acetamide;
2-(2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-{6′-fluoro-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
2-{(3′R,4S)-3′,6′-difluoro-5′-[(methylcarbamoyl)amino]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
methyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate;
{4-[(4-fluorobenzyl) {[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
N-(4-fluorobenzyl)-2-[(4R)-7′-hydroxy-2,5-dioxo-2′,3′-dihydro-1H-spiro[imidazolidine-4,1′-inden]-1-yl]-N-[(2S)-1,1,1-trifluoropropan-2-yl]acetamide;
tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetate;
{4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetic acid;
2-[(4S)-2,5-dioxo-5′-(1H-pyrazol-5-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
4-[(4-fluorobenzyl) {[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexanecarboxylic acid;
2-[(4S)-5′-{1-[2-(dimethylamino)ethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
tert-butyl 4-({4-[(4S)-1-(2-{(4-fluorobenzyl)[(2S)-1,1,1-trifluoropropan-2-yl]amino}-2-oxoethyl)-2,5-dioxo-2′,3′-dihydrospiro[imidazolidine-4,1′-inden]-5′-yl]-1H-pyrazol-1-yl}methyl)piperidine-1-carboxylate;
2-{(4S)-2,5-dioxo-5′-[1-(piperidin-4-ylmethyl)-1H-pyrazol-4-yl]-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-{6-[(1-methyl-1H-pyrazol-4-yl)amino]-1,1-dioxido-2′,5′-dioxo-1′H-spiro[1-benzothiophene-3,4′-imidazolidin]-1′-yl}-N-[(2S)-1,1,1-trifluoropropan-2-yl]acetamide;
N-(4-fluorobenzyl)-2-(6-methoxy-1,1-dioxido-2′,5′-dioxo-1′H-spiro[1-benzothiophene-3,4′-imidazolidin]-1′-yl)-N-[(2S)-1,1,1-trifluoropropan-2-yl]acetamide;
N-(4-fluorobenzyl)-2-[(3 S)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide;
N-(4-fluorobenzyl)-2-[(3R)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide;
N-benzyl-N-(1-cyclopropylethyl)-2-(2,5-dioxo-2′,3′-dihydro-1H-spiro[imidazolidine-4,1′-inden]-1-yl)acetamide;
N-(1-cyclopropylethyl)-2-(2,5-dioxo-2′,3′-dihydro-1H-spiro[imidazolidine-4,1′-inden]-1-yl)-N-(1H-imidazol-4-ylmethyl)acetamide;
N-benzyl-N-(1-cyclopropylethyl)-2-(2,2-dimethyl-2′,5′-dioxo-1′H-spiro[1-benzofuran-3,4′-imidazolidin]-1′-yl)acetamide;
N-benzyl-N-(1-cyclopropylethyl)-2-(1′,3-dimethyl-2,2′,5-trioxo-1′,2′-dihydro-1H-spiro[imidazolidine-4,3′-indol]-1-yl)acetamide;
N-benzyl-N-(1-cyclopropylethyl)-2-(2,5-dioxo-2′,3′-dihydro-1H-spiro[imidazolidine-4,1′-inden]-1-yl)-2-methylpropanamide;
N-benzyl-N-(1-cyclopropylethyl)-2-[2,5-dioxo-3-(prop-2-en-1-yl)-2′,3′-dihydro-1H-spiro[imidazolidine-4,1′-inden]-1-yl]acetamide;
N-(1-cyclopropylethyl)-2-(2,5-dioxo-2′,3′-dihydro-1H-spiro[imidazolidine-4,1′-inden]-1-yl)-N-(2,2,2-trifluoroethyl)acetamide;
N-(1-cyclopropylethyl)-2-(2,5-dioxo-2′,3′-dihydro-1H-spiro[imidazolidine-4,1′-inden]-1-yl)-N-[3-(trifluoromethoxy)benzyl]acetamide;
N-(but-2-yn-1-yl)-N-(1-cyclopropylethyl)-2-(2,5-dioxo-2′,3′-dihydro-1H-spiro[imidazolidine-4,1′-inden]-1-yl)acetamide;
N-(1-cyclopropylethyl)-N-{[6-(difluoromethoxy)naphthalen-2-yl]methyl}-2-(2,5-dioxo-2′,3′-dihydro-1H-spiro[imidazolidine-4,1′-inden]-1-yl)acetamide;
N-benzyl-N-(1-cyclopropylethyl)-2-[5′-(methylsulfonyl)-2,5-dioxo-2′,3′-dihydro-1H-spiro[imidazolidine-4,1′-inden]-1-yl]acetamide;
N-benzyl-N-(1-cyclobutylethyl)-2-(7′-methoxy-2,5-dioxo-2′,3′-dihydro-1H-spiro[imidazolidine-4,1′-inden]-1-yl)acetamide;
N-benzyl-N-(1-cyclopropylethyl)-2-[5′-(hydroxymethyl)-3-methyl-2,5-dioxo-2′,3′-dihydro-1H-spiro[imidazolidine-4,1′-inden]-1-yl]acetamide;
N-[(1S)-1-cyclobutylethyl]-2-[(4R)-2,5-dioxo-2′,3′-dihydro-1H-spiro[imidazolidine-4,1′-inden]-1-yl]-N-(prop-2-yn-1-yl)acetamide;
N³-[(1S)-1-cyclobutylethyl]-N³-{[(4R)-2,5-dioxo-2′,3′-dihydro-1H-spiro[imidazolidine-4,1′-inden]-1-yl]acetyl}-beta-alaninamide;
N-(biphenyl-4-ylmethyl)-N-[(1S)-1-cyclobutylethyl]-2-[(4R)-2,5-dioxo-2′,3′-dihydro-1H-spiro[imidazolidine-4,1′-inden]-1-yl]acetamide;
N²-[(1S)-1-cyclobutylethyl]-N²-{[(4R)-2,5-dioxo-2′,3′-dihydro-1H-spiro[imidazolidine-4,1′-inden]-1-yl]acetyl}glycinamide;
N-[(1S)-1-cyclobutylethyl]-2-[(4R)-2,5-dioxo-2′,3′-dihydro-1H-spiro[imidazolidine-4,1′-inden]-1-yl]-N-[(1-methyl-2-oxo-1,2-dihydropyridin-4-yl)methyl]acetamide;
N-benzyl-N-[(1R)-1-cyclopropylethyl]-2-[(4R)-2,5-dioxo-2′,3′-dihydro-1H-spiro[imidazolidine-4,1′-inden]-1-yl]acetamide;
N-benzyl-N-[(1S)-1-cyclopropylethyl]-2-[(4S)-2,5-dioxo-2′,3′-dihydro-1H-spiro[imidazolidine-4,1′-inden]-1-yl]acetamide;
N-benzyl-N-[(1S)-1-cyclopropylethyl]-2-[(4R)-2,5-dioxo-2′,3′-dihydro-1H-spiro[imidazolidine-4,1′-inden]-1-yl]acetamide;
N-benzyl-N-[(1R)-1-cyclopropylethyl]-2-[(4 S)-2,5-dioxo-2′,3′-dihydro-1H-spiro[imidazolidine-4,1′-inden]-1-yl]acetamide;
N-[4-(acetylamino)benzyl]-N-[(1S)-1-cyclobutylethyl]-2-[(4R)-2,5-dioxo-2′,3′-dihydro-1H-spiro[imidazolidine-4,1′-inden]-1-yl]acetamide;
N-[(1S)-1-cyclobutylethyl]-2-[(4R)-2,5-dioxo-2′,3′-dihydro-1H-spiro[imidazolidine-4,1′-inden]-1-yl]-N-(4-ethynylbenzyl)acetamide;
N-[(1S)-1-cyclobutylethyl]-2-[(4R)-2,5-dioxo-2′,3′-dihydro-1H-spiro[imidazolidine-4,1′-inden]-1-yl]-N-({1-[2-(methylamino)-2-oxoethyl]-1H-1,2,3-triazol-4-yl}methyl)acetamide;
2-{4-[([(1S)-1-cyclobutylethyl]{[(4R)-2,5-dioxo-2′,3′-dihydro-1H-spiro[imidazolidine-4, 1′-inden]-1-yl]acetyl}amino)methyl]-1H-1,2,3-triazol-1-yl}-N,N-dimethylacetamide;
N³-[(1S)-1-cyclobutylethyl]-N³-{[(4R)-2,5-dioxo-2′,3′-dihydro-1H-spiro[imidazolidine-4,1′-inden]-1-yl]acetyl}-N-methyl-beta-alaninamide;
N-benzyl-N-(1-cyclopropylethyl)-2-[5′-(6-hydroxypyridin-3-yl)-2,5-dioxo-2′,3′-dihydro-1H-spiro[imidazolidine-4,1′-inden]-1-yl]acetamide;
N-benzyl-N-[(1S)-1-cyclopropylethyl]-2-[5′-(formylamino)-2,5-dioxo-2′,3′-dihydro-1H-spiro[imidazolidine-4,1′-inden]-1-yl]acetamide;
N-benzyl-2-[(4R)-5′-bromo-2,5-dioxo-2′,3′-dihydro-1H-spiro[imidazolidine-4,1′-inden]-1-yl]-N-[(1S)-1-cyclopropylethyl]acetamide;
2-[1,1-dioxido-2′,5′-dioxo-6-(pyridin-3-yl)-1H-spiro[1-benzothiophene-3,4′-imidazolidin]-1′-yl]-N-(4-fluorobenzyl)-N-[(2S)-1,1,1-trifluoropropan-2-yl]acetamide;
N-(4-fluorobenzyl)-2-[(3′S,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
(4-{[(5′-bromo-6′-fluoro-2,5-dioxo-2′,3′-dihydro-1H-spiro[imidazolidine-4,1′-inden]-1-yl)acetyl](4-fluorobenzyl)amino}cyclohexylidene)acetic acid;
{4-[(4-fluorobenzyl) {[6′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
tert-butyl (4-{[(5′-bromo-6′-fluoro-2,5-dioxo-2′,3′-dihydro-1H-spiro[imidazolidine-4,1′-inden]-1-yl)acetyl](4-fluorobenzyl)amino}cyclohexylidene)acetate; and pharmaceutically acceptable salts thereof.

In another aspect, the present invention relates to compounds of Formula (XIIa) or (XIIb),

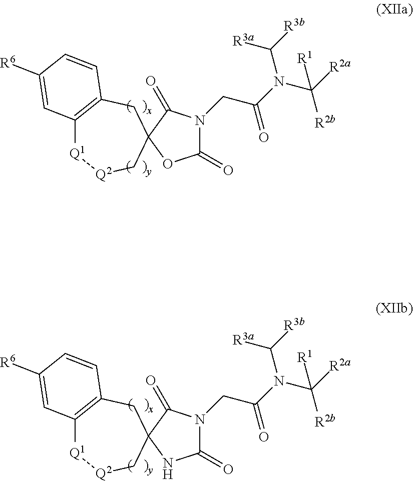

or a stereoisomer, tautomer or pharmaceutically acceptable salt thereof,
wherein
[symbol] is —C(R¹⁰)₂—C(R¹⁴)₂—, —O—C(R¹⁴)₂—, —O—C(O)—, —S(O)₂—C(R¹⁴)₂—, —S—C(R¹⁴)₂—, —NR⁹—C(O)—, —NR⁹—C(R¹⁴)₂—, —C(R¹⁰)₂—O—, —C(R¹⁰)₂—, or —C(R¹⁰)═C(R¹⁴)—;
R¹ is carbocyclyl or heterocyclyl;
R^{2a} and R^{2b} are each independently H, D, or C₁-C₆ alkyl;
R^{3a} is hydrogen, C(O)NH₂, C₁-C₆ alkyl, aryl, cycloalkyl or heterocyclyl; and
R^{3b} is C₁-C₆ alkyl, aryl, cycloalkyl or heterocyclyl; or
R^{3a} and R^{3b} taken together with the carbon to which they are attached form an arene, cycloalkane, or heterocycle;
R⁶ is independently halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═S)N(R¹²)₂, —NHC(═N—C≡N)NR¹², —NHC(═N—C≡N)SR¹², or —NHS(O)ₘR¹²;
R⁹ is independently H or C₁-C₆ alkyl;
R¹⁰, at each occurrence, is independently H, —OH, halo, —CN, —CO₂R¹², —C(═O)NHR¹³, —NHR¹², C₁-C₆ alkyl or alkoxy; or two R¹⁰ taken together form oxo or ═N—OR¹¹;
R¹¹ and R¹³ are each independently H, —OH, or C₁-C₆ alkyl;
R¹², at each occurrence, is independently H, C₁-C₆ alkyl, aryl, cycloalkyl, or heterocyclyl;
R¹⁴, at each occurrence, is independently H or C₁-C₆ alkyl;
m, at each occurrence, is independently 0, 1 or 2; and
x, and y are each independently 0 or 1, wherein x and y are selected such that the sum of x+y is 0 or 1; and

with the proviso that R^{3a} and R^{3b} taken together with the carbon to which they are attached do not form tetrahydrothiophene 1,1-dioxide or tetrahydrothiophene when R¹ is unsubstituted phenyl.

In one embodiment of Formula (XIIa) or (XIIb), [symbol] is —C(R¹⁰)₂—C(R¹⁴)₂—, —O—C(R¹⁴)₂—, —O—C(O)—, —S(O)₂—C(R¹⁴)₂—, —S—C(R¹⁴)₂—, —NR⁹—C(O)—, —NR⁹—C(R¹⁴)₂—, —C(R¹⁰)₂—O—, —C(R¹⁰)₂—, or —C(R¹⁰)═C(R¹⁴)—. In another embodiment of Formula (XIIa) or (XIIb), [symbol] is C(R¹⁰)₂—C(R¹⁴)₂—. In another embodiment of Formula (XIIa) or (XIIb), [symbol] is —O—C(R¹⁴)₂—. In another embodiment of Formula (XIIa) or (XIIb), [symbol] is —O—C(O)—. In another embodiment of Formula (XIIa) or (XIIb), [symbol] is —S(O)₂—C(R¹⁴)₂—. In another embodiment of Formula (XIIa) or (XIIb), [symbol] is —S—C(R¹⁴)₂—. In another embodiment of Formula (XIIa) or (XIIb), [symbol] is —NR⁹—C(O)—. In another embodiment of Formula (XIIa) or (XIIb), [symbol] is —NR⁹—C(R¹⁴)₂—. In another embodiment of Formula (XIIa) or (XIIb), [symbol] is —C(R¹⁰)₂—O—. In another embodiment of Formula (XIIa) or (XIIb), [symbol] is —C(R¹⁰)₂—. In another embodiment of Formula (XIIa) or (XIIb), [symbol] is —C(R¹⁰)═C(R¹⁴)—.

In one embodiment of Formula (XIIa) or (XIIb), R¹ is carbocyclyl or heterocyclyl. In another embodiment of Formula (XIIa) or (XIIb), R¹ is carbocyclyl. In another embodiment of Formula (XIIa) or (XIIb), R¹ is heterocyclyl. In another embodiment of Formula (XIIa) or (XIIb), R¹ is phenyl, naphthyl, cyclopropyl, or cyclobutyl. In another embodiment of Formula (XIIa) or (XIIb), R¹ is phenyl. In another embodiment of Formula (XIIa) or (XIIb), R¹ is phenyl, which is unsubstituted. In another embodiment of Formula (XIIa) or (XIIb), R¹ is phenyl, which is substituted. In another embodiment of Formula (XIIa) or (XIIb), R¹ is phenyl, which is substituted with F, Cl, Br, CN, —NR_{g}Rₕ, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, aryl, and/or haloalkyl; wherein alkyl, alone or part of a group, is optionally substituted with F, C(═O)NR_{g}Rₕ, or CN. In another embodiment of Formula (XIIa) or (XIIb), R¹ is phenyl, which is substituted with F, Cl, Br, CN, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, and/or haloalkyl; wherein alkyl, alone or part of a group, is optionally substituted with F. In another embodiment of Formula (XIIa) or (XIIb), R¹ is phenyl, which is substituted with F. In another embodiment of Formula (XIIa) or (XIIb), R¹ is tetrahydropyranyl, thiazolyl, oxazolyl, thiadiazolyl, pyrimidinyl, thiophenyl, furanyl, triazolyl, indolyl, imidazolyl, or pyridinyl. In another embodiment of Formula (XIIa) or (XIIb), R¹ is tetrahydropyranyl, thiazolyl, oxazolyl, thiadiazolyl, pyrimidinyl, thiophenyl, furanyl, triazolyl, indolyl, imidazolyl, or pyridinyl; which is unsubstituted. In another embodiment of Formula (XIIa) or (XIIb), R¹ is tetrahydropyranyl, thiazolyl, oxazolyl, thiadiazolyl, pyrimidinyl, thiophenyl, furanyl, triazolyl, indolyl, imidazolyl, or pyridinyl; which is substituted. In another embodiment of Formula (XIIa) or (XIIb), R¹ is tetrahydropyranyl, thiazolyl, oxazolyl, thiadiazolyl, pyrimidinyl, thiophenyl, furanyl, triazolyl, indolyl, imidazolyl, or pyridinyl; which is substituted with F, Cl, Br, CN, —NR_{g}Rₕ, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, aryl, and/or haloalkyl; wherein alkyl, alone or part of a group, is optionally substituted with F, C(═O)NR_{g}Rₕ, or CN.

In one embodiment of Formula (XIIa) or (XIIb), R^{2a} and R^{2b} are each independently H, D, or C₁-C₆ alkyl. In another embodiment of Formula (XIIa) or (XIIb), R^{2a} and R^{2b} are each independently H. In another embodiment of Formula (XIIa) or (XIIb), R^{2a} is H and R^{2b} is C₁-C₆ alkyl. In another embodiment of Formula (XIIa) or (XIIb), R^{2a} is H and R^{2b} is C₁-alkyl.

In one embodiment of Formula (XIIa) or (XIIb), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; or R^{3a} and R^{3b} taken together with the carbon to which they are attached form arene, cycloalkane, or heterocycle. In another embodiment of Formula (XIIa) or (XIIb), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; wherein C₁-C₆ alkyl, is optionally substituted with F, —CN, —NR_{g}Rₕ, —OR_{g}, or —SO₂R_{g}; wherein R_{g} and Rₕ are the same or different and independently hydrogen or alkyl; wherein aryl, cycloalkyl and heterocyclyl are optionally substituted with alkyl, —SO₂NR_{g}C(═O)ORₕ, —SO₂NR_{g}Rₕ, —C(═O)R_{g}, —C(═O)OR_{g}, or —C(═O)NR_{g}Rₕ; wherein R_{g} and Rₕ are the same or different and independently hydrogen or alkyl; wherein alkyl, alone or part of a group, is optionally substituted with aryl or —C(═O)OH.

In another embodiment of Formula (XIIa) or (XIIb), R^{3a} is C₁-C₆ alkyl; and R^{3b} is C₁-C₆ alkyl; wherein the C₁-C₆ alkyl is optionally substituted with one or more F, —CN, alkoxy, or SO₂R_{g}. In another embodiment of Formula (XIIa) or (XIIb), R^{3a} is C₁-C₆ alkyl; and R^{3b} is CF₃. In another embodiment of Formula (XIIa) or (XIIb), R^{3a} is CH₃; and R^{3b} is CF₃. In another embodiment of Formula (XIIa) or (XIIb), R^{3a} is hydrogen; and R^{3b} is C₁-C₆ alkyl; wherein the C₁-C₆ alkyl is optionally substituted with one or more F, —CN, alkoxy, or SO₂R_{g}. In another embodiment of Formula (XIIa) or (XIIb), R^{3a} is hydrogen; and R^{3b} is cycloalkyl. In another embodiment of Formula (XIIa) or (XIIb), R^{3a} is hydrogen; and R^{3b} is cyclopropyl. In another embodiment of Formula (XIIa) or (XIIb), R^{3a} is C₁-C₆ alkyl; wherein the C₁-C₆ alkyl is CH₃; and R^{3b} is cycloalkyl. In another embodiment of Formula (XIIa) or (XIIb), R^{3a} is CH₃; and R^{3b} is cyclopropyl. In another embodiment of Formula (XIIa) or (XIIb), R^{3a} is CF₃; and R^{3b} is cyclopropyl. In another embodiment of Formula (XIIa) or (XIIb), R^{3a} is cycloalkyl; and R^{3b} is cycloalkyl. In another embodiment of Formula (XIIa) or (XIIb), R^{3a} is cyclopropyl; and R^{3b} is cyclopropyl. In another embodiment of Formula (XIIa) or (XIIb), R^{3a} is C₁-C₆ alkyl; and R^{3b} is heterocyclyl. In another embodiment of Formula (XIIa) or (XIIb), R^{3a} is hydrogen; and R^{3b} is aryl. In another embodiment of Formula (XIIa) or (XIIb), R^{3a} and R^{3b} taken together with the carbon to which they are attached form arene, cycloalkane, or heterocycle. In another embodiment of Formula (XIIa) or (XIIb), R^{3a} and R^{3b} taken together with the carbon to which they are attached form arene. In another embodiment of Formula (XIIa) or (XIIb), R^{3a} and R^{3b} taken together with the carbon to which they are attached form cycloalkane. In another embodiment of Formula (XIIa) or (XIIb), R^{3a} and R^{3b} taken together with the carbon to which they are attached form heterocycle. In another embodiment of Formula (XIIa) or (XIIb), R^{3a} and R^{3b} taken together with the carbon to which they are attached form cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, or benzene. In another embodiment of Formula (XIIa) or (XIIb), R^{3a} and R^{3b} taken together with the carbon to which they are attached form cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, or benzene wherein cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, and benzene are optionally substituted with alkyl, haloalkyl, F, CN, —NR_{g}Rₕ, —NR_{g}C(═O)Rₕ, —NR_{g}SO₂Rₕ, —OR_{g}, —SO₂R_{g}, —NR_{g}SO₂NR_{g}C(═O)Rₕ, —SO₂NR_{g}C(═O)ORₕ—SO₂NR_{g}Rₕ, —C(═O)R_{g}, —C(═O)OR_{g}, or —C(═O)NR_{g}Rₕ; wherein R_{g} and Rₕ are the same or different and independently hydrogen, alkyl, or haloalkyl; wherein each alkyl, alone or part of a group, is optionally substituted with CN, alkyl, —SO₂NH₂, C(═O)NHCH₃, —C(═O)OC(CH₃)₃, —C(═O)OCH₃, or —C(═O)OH.

In one embodiment of Formula (XIIa) or (XIIb), R⁶ is independently halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R²)₂, —NHC(═S)N(R¹²)₂, —NHC(═N—C≡N)NR¹², —NHC(═N—C≡N)SR¹², or —NHS(O)ₘR¹². In another embodiment of Formula (XIIa) or (XIIb), R⁶ is halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R²)₂, —NHC(═S)N(R¹²)₂, —NHC(═N—C≡N)NR¹², —NHC(═N—C≡N)SR¹², or —NHS(O)ₘR¹². In another embodiment of Formula (XIIa) or (XIIb), R⁶ is —N(R¹²)₂, —C(═O)NHR¹², —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═O)N(R¹²)₂, —NHC(═S)N(R¹²)₂, —NHC(═N—C≡N)NR¹², —NHC(═N—C≡N)SR¹², or —NHS(O)ₘR¹². In another embodiment of Formula (XIIa) or (XIIb), R⁶ is heterocyclyl. In another embodiment of Formula (XIIa) or (XIIb), R⁶ is pyrazolyl. In another embodiment of Formula (XIIa) or (XIIb), R⁶ is —NHC(═O)NHR¹².

In one embodiment of Formula (XIIa) or (XIIb), R⁹ is independently H or C₁-C₆ alkyl. In another embodiment of Formula (XIIa) or (XIIb), R⁹ is independently H. In another embodiment of Formula (XIIa) or (XIIb), R⁹ is independently C₁-C₆ alkyl.

In one embodiment of Formula (XIIa) or (XIIb), R¹⁰, at each occurrence, is independently H, —OH, halo, —CN, —CO₂R¹², —C(═O)NHR¹³, —NHR¹², C₁-C₆ alkyl or alkoxy; or two R¹⁰ taken together form oxo or ═N—OR¹¹. In another embodiment of Formula (XIIa) or (XIIb), R¹⁰, at each occurrence, is independently H, —OH, halo, —CN, —CO₂R¹², —C(═O)NHR¹³, —NHR¹², C₁-C₆ alkyl or alkoxy; or two R¹⁰ taken together form oxo or ═N—OR¹¹. In another embodiment of Formula (XIIa) or (XIIb), R¹⁰, at each occurrence, is independently H. In another embodiment of Formula (XIIa) or (XIIb), R¹⁰, at each occurrence, is independently H or —OH. In another embodiment of Formula (XIIa) or (XIIb), R¹⁰, at each occurrence, is independently H or halo. In another embodiment of Formula (XIIa) or (XIIb), R¹⁰, at each occurrence, is independently H or F. In another embodiment of Formula (XIIa) or (XIIb), one R¹⁰ is independently H, and the remaining is F. In another embodiment of Formula (XIIa) or (XIIb), R¹⁰, at each occurrence, is independently H or —CN. In another embodiment of Formula (XIIa) or (XIIb), R¹⁰, at each occurrence, is independently H or —CO₂R¹². In another embodiment of Formula (XIIa) or (XIIb), R¹⁰, at each occurrence, is independently H or —C(═O)NHR¹³. In another embodiment of Formula (XIIa) or (XIIb), R¹⁰, at each occurrence, is independently H or —NHR¹². In another embodiment of Formula (XIIa) or (XIIb), R¹⁰, at each occurrence, is independently H or C₁-C₆ alkyl. In another embodiment of Formula (XIIa) or (XIIb), R¹⁰, at each occurrence, is independently H or alkoxy. In another embodiment of Formula (XIIa) or (XIIb), two R¹⁰ taken together form oxo. In another embodiment of Formula (XIIa) or (XIIb), two R¹⁰ taken together form ═N—OR¹¹.

In one embodiment of Formula (XIIa) or (XIIb), R¹¹ and R¹³ are each independently H, —OH, or C₁-C₆ alkyl. In another embodiment of Formula (XIIa) or (XIIb), R¹¹ and R¹³ are each independently H. In another embodiment of Formula (XIIa) or (XIIb), R¹¹ and R¹³ are each independently —OH. In another embodiment of Formula (XIIa) or (XIIb), R¹¹ and R¹³ are each independently C₁-C₆ alkyl.

In one embodiment of Formula (XIIa) or (XIIb), R¹², at each occurrence, is independently H, C₁-C₆ alkyl, aryl, cycloalkyl, or heterocyclyl. In another embodiment of Formula (XIIa) or (XIIb), R¹², at each occurrence, is independently H, C₁-C₆ alkyl, aryl, cycloalkyl, or heterocyclyl; wherein C₁-C₆ alkyl, aryl, cycloalkyl, and heterocyclyl are optionally substituted with F, alkyl, alkoxy, cycloalkyl, haloalkyl, heterocyclyl, heterocyclylalkyl, oxo, CN, —NR_{g}Rₕ, —NR_{g}C(═O)Rₕ, —NR_{g}C(═O)ORₕ, —OR_{g}, —SO₂R_{g}, —SO₂NR_{g}Rₕ, —C(═O)R_{g}, —C(═O)OR_{g}, or —C(═O)NR_{g}Rₕ; wherein R_{g} and Rₕ are the same or different and independently hydrogen, alkyl, alkoxy, aryl, cycloalkyl, and/or haloalkyl.

In one embodiment of Formula (XIIa) or (XIIb), R¹⁴, at each occurrence, is independently H or C₁-C₆ alkyl. In another embodiment of Formula (XIIa) or (XIIb), R¹⁴, at each occurrence, is independently H. In another embodiment of Formula (XIIa) or (XIIb), R¹⁴, at each occurrence, is independently C₁-C₆ alkyl.

In one embodiment of Formula (XIIa) or (XIIb), m, at each occurrence, is independently 0, 1 or 2. In another embodiment of Formula (XIIa) or (XIIb), m, at each occurrence, is independently 0. In another embodiment of Formula (XIIa) or (XIIb), m, at each occurrence, is independently 1. In another embodiment of Formula (XIIa) or (XIIb), m, at each occurrence, is independently 2.

In one embodiment of Formula (XIIa) or (XIIb), x, and y are each independently 0 or 1, wherein x and y are selected such that the sum of x+y is 0 or 1. In another embodiment of Formula (XIIa) or (XIIb), x is 0 and y is 0. In another embodiment of Formula (XIIa) or (XIIb), x is 0 and y is 1. In another embodiment of Formula (XIIa) or (XIIb), x is 1 and y is 0.

Still another embodiment pertains to compounds of Formula (XIIa), selected from the group consisting of:
N-benzyl-2-(5-bromo-2′,4′-dioxo-2,3-dihydrospiro[indene-1,5′-ozazolidine]-3′-yl)-N-(1-cyclopropylethyl)acetamide;
2-(5-bromo-2′,4′-dioxo-2,3-dihydrospiro[indene-1,5′-ozazolidine]-3′-yl)-N-(1-cyclopropylethyl)-N-(4-fluorobenzyl)acetamide;
N-benzyl-2-(5-cyano-2′,4′-dioxo-2,3-dihydrospiro[indene-1,5′-ozazolidine]-3′-yl)-N-(1-cyclopropylethyl)acetamide;
(S)-2-(5-acetamido-2′,4′-dioxo-2,3-dihydrospiro[indene-1,5′-ozazolidine]-3′-yl)-N-benzyl-N-(1-cyclopropylethyl)acetamide;
3′-(2-(benzyl(1-cyclopropylethyl)amino)-2-oxoethyl)-2′,4′-dioxo-2,3-dihydrospiro[indene-1,5′-ozazolidine]-5-carboxamide;
N-benzyl-N-(1-cyclopropylethyl)-2-(5-methoxy-2′,4′-dioxo-2,3-dihydrospiro[indene-1,5′-ozazolidine]-3′-yl)acetamide;
N-benzyl-N—((S)-1-cyclopropylethyl)-2-(5-(3-hydroxyprop-1-ynyl)-2′,4′-dioxo-2,3-dihydrospiro[indene-1,5′-ozazolidine]-3′-yl)acetamide;
N-benzyl-N—((S)-1-cyclopropylethyl)-2-(5-(methylsulfonamido)-2′,4′-dioxo-2,3-dihydrospiro[indene-1,5′-ozazolidine]-3′-yl)acetamide;
(S)-3′-(2-(benzyl(1-cyclopropylethyl)amino)-2-oxoethyl)-2′,4′-dioxo-2,3-dihydrospiro[indene-1,5′-ozazolidine]-5-carboxamide;
N-benzyl-N—((S)-1-cyclopropylethyl)-2-(5-(morpholinomethyl)-2′,4′-dioxo-2,3-dihydrospiro[indene-1,5′-ozazolidine]-3′-yl)acetamide;
N-benzyl-N—((S)-1-cyclopropylethyl)-2-(5-(3-methylureido)-2′,4′-dioxo-2,3-dihydrospiro[indene-1,5′-ozazolidine]-3′-yl)acetamide;
(S)—N-(3′-(2-(benzyl(1-cyclopropylethyl)amino)-2-oxoethyl)-2′,4′-dioxo-2,3-dihydrospiro[indene-1,5′-ozazolidine]-5-yl)-3-oxobutanamide;
N-benzyl-N—((S)-1-cyclopropylethyl)-2-(5-morpholino-2′,4′-dioxo-2,3-dihydrospiro[indene-1,5′-ozazolidine]-3′-yl)acetamide;
N-benzyl-2-(5-(2-cyanoacetamido)-2′,4′-dioxo-2,3-dihydrospiro[indene-1,5′-ozazolidine]-3′-yl)-N—((S)-1-cyclopropylethyl)acetamide;
N-benzyl-N—((S)-1-cyclopropylethyl)-2-(2′,4′-dioxo-5-(2-oxa-6-azaspiro[3.3]heptan-6-yl)-2,3-dihydrospiro[indene-1,5′-ozazolidine]-3′-yl)acetamide;
N-benzyl-N—((S)-1-cyclopropylethyl)-2-(2′,4′-dioxo-5-(piperidin-1-ylmethyl)-2,3-dihydrospiro[indene-1,5′-ozazolidine]-3′-yl)acetamide;
N-benzyl-N—((S)-1-cyclopropylethyl)-2-(2′,4′-dioxo-5-(piperazin-1-ylmethyl)-2,3-dihydrospiro[indene-1,5′-ozazolidine]-3′-yl)acetamide;
N-benzyl-N—((S)-1-cyclopropylethyl)-2-(5-((4-hydroxypiperidin-1-yl)methyl)-2′,4′-dioxo-2,3-dihydrospiro[indene-1,5′-ozazolidine]-3′-yl)acetamide;
(S)-2-amino-N-(3′-(2-(benzyl(1-cyclopropylethyl)amino)-2-oxoethyl)-2′,4′-dioxo-2,3-dihydrospiro[indene-1,5′-ozazolidine]-5-yl)-2-methylpropanamide;
(S)—N-benzyl-N-(1-cyclopropylethyl)-2-(2′,4′-dioxo-5-(piperazin-1-yl)-2,3-dihydrospiro[indene-1,5′-ozazolidine]-3′-yl)acetamide;
N-benzyl-N—((S)-1-cyclopropylethyl)-2-(5-hydroxy-2′,4′-dioxo-2,3-dihydrospiro[indene-1,5′-ozazolidine]-3′-yl)acetamide;
methyl 2-(3′-(2-(benzyl((S)-1-cyclopropylethyl)amino)-2-oxoethyl)-2′,4′-dioxo-2,3-dihydrospiro[indene-1,5′-ozazolidine]-5-yloxyacetate;
N-benzyl-N—((S)-1-cyclopropylethyl)-2-(2′,4′-dioxo-5-(prop-2-ynyloxy)-2,3-dihydrospiro[indene-1,5′-ozazolidine]-3′-yl)acetamide;
(S)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)-3-methylbutanamide;
(2S)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)propanamide;
2-(3′-(2-(benzyl((S)-1-cyclopropylethyl)amino)-2-oxoethyl)-2′,4′-dioxo-2,3-dihydrospiro[indene-1,5′-ozazolidine]-5-ylamino)acetic acid;
N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-cyano-3-oxobutanamide;
2-(3′-(2-(benzyl((S)-1-cyclopropylethyl)amino)-2-oxoethyl)-2′,4′-dioxo-2,3-dihydrospiro[indene-1,5′-ozazolidine]-5-yloxy)acetic acid;
N-benzyl-N—((S)-1-cyclopropylethyl)-2-(5-(oxetan-3-ylamino)-2′,4′-dioxo-2,3-dihydrospiro[indene-1,5′-ozazolidine]-3′-yl)acetamide;
N-benzyl-N—((S)-1-cyclopropylethyl)-2-(2′,4′-dioxo-5-(tetrahydro-2H-pyran-4-ylamino)-2,3-dihydrospiro[indene-1,5′-ozazolidine]-3′-yl)acetamide;
N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-hydroxypropanamide;
1-(3′-(2-(benzyl((S)-1-cyclopropylethyl)amino)-2-oxoethyl)-2′,4′-dioxo-2,3-dihydrospiro[indene-1,5′-ozazolidine]-5-yl)azetidine-3-carboxamide;
2-amino-N-(3′-(2-(benzyl(dicyclopropylmethyl)amino)-2-oxoethyl)-2′,4′-dioxo-2,3-dihydrospiro[indene-1,5′-ozazolidine]-5-yl)-2-methylpropanamide;
N-benzyl-N-(dicyclopropylmethyl)-2-(5-(3-methylureido)-2′,4′-dioxo-2,3-dihydrospiro[indene-1,5′-ozazolidine]-3′-yl)acetamide;
N-benzyl-N—((S)-1-cyclopropylethyl)-2-(5-(2-(dimethylamino)ethoxy)-2′,4′-dioxo-2,3-dihydrospiro[indene-1,5′-ozazolidine]-3′-yl)acetamide;
2-(5-((1H-1,2,3-triazol-4-yl)methoxy)-2′,4′-dioxo-2,3-dihydrospiro[indene-1,5′-ozazolidine]-3′-yl)-N-benzyl-N—((S)-1-cyclopropylethyl)acetamide;
N-benzyl-N—((S)-1-cyclopropylethyl)-2-(2′,4′-dioxo-5-(6-oxo-1,6-dihydropyridin-3-yl)-2,3-dihydrospiro[indene-1,5′-ozazolidine]-3′-yl)acetamide;
N-benzyl-N—((S)-1-cyclopropylethyl)-2-(5-(2-hydroxyethoxy)-2′,4′-dioxo-2,3-dihydrospiro[indene-1,5′-ozazolidine]-3′-yl)acetamide;
3′-(2-(benzyl((S)-1-cyclopropylethyl)amino)-2-oxoethyl)-2′,4′-dioxo-2,3-dihydrospiro[indene-1,5′-ozazolidine]-5-ylboronic acid;
N-benzyl-N—((S)-1-cyclopropylethyl)-2-(5-(3-(hydroxymethyl)azetidin-1-yl)-2′,4′-dioxo-2,3-dihydrospiro[indene-1,5′-ozazolidine]-3′-yl)acetamide;
N—((S)-1-cyclopropylethyl)-2-(2′,4′-dioxo-5-(6-oxo-1,6-dihydropyridin-3-yl)-2,3-dihydrospiro[indene-1,5′-ozazolidine]-3′-yl)-N-(4-fluorobenzyl)acetamide;
1-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)cyclobutanecarboxamide;
2-(5-(2-cyanoacetamido)-2′,4′-dioxo-2,3-dihydrospiro[indene-1,5′-ozazolidine]-3′-yl)-N—((S)-1-cyclopropylethyl)-N-(4-fluorobenzyl)acetamide;
N—((S)-1-cyclopropylethyl)-N-(4-fluorobenzyl)-2-(5-(3-methylureido)-2′,4′-dioxo-2,3-dihydrospiro[indene-1,5′-ozazolidine]-3′-yl)acetamide;
3′-((4S)-3-benzyl-4-cyclopropyl-2-oxopentyl)-5-(1H-pyrazol-5-yl)-2,3-dihydrospiro[indene-1,5′-ozazolidine]-2′,4′-dione;
2-amino-N-(3′-(2-(benzyl((S)-1-cyclopropylethyl)amino)-2-oxoethyl)-4′-oxo-2,3-dihydrospiro[indene-1,5′-ozazolidine]-5-yl)-3-hydroxypropanamide;
(S)—N-(3′-(2-(benzyl(1-cyclopropylethyl)amino)-2-oxoethyl)-2′,4′-dioxo-2,3-dihydrospiro[indene-1,5′-ozazolidine]-5-yl)-2-hydroxy-2-methylpropanamide;
1-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)cyclopropanecarboxamide;
2-amino-N-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)-2-methylpropanamide;
(S)-2-(5-(azetidin-3-ylamino)-2′,4′-dioxo-2,3-dihydrospiro[indene-1,5′-ozazolidine]-3′-yl)-N-benzyl-N-(1-cyclopropylethyl)acetamide;
(2R)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-ylpropanamide;
(S)-2-(3′-(2-(benzyl(1-cyclopropylethyl)amino)-2-oxoethyl)-2′,4′-dioxo-2,3-dihydrospiro[indene-1,5′-ozazolidine]-5-ylcarboxamido)acetic acid;
2-((S)-5-acetamido-2′,4′-dioxo-2,3-dihydrospiro[indene-1,5′-ozazolidine]-3′-yl)-N-benzyl-N—((S)-1-cyclopropylethyl)acetamide;
2-((R)-5-acetamido-2′,4′-dioxo-2,3-dihydrospiro[indene-1,5′-ozazolidine]-3′-yl)-N-benzyl-N—((S)-1-cyclopropylethyl)acetamide;
N-benzyl-2-{5-[(methylcarbamoyl)amino]-3′,5′-dioxo-2,3-dihydrospiro[indene-1,2′-[1,4]ozazolidine]-4′-yl}-N-(pentan-3-yl)acetamide;
2-amino-N-(4′-{[benzyl(pentan-3-yl)carbamoyl]methyl}-3′,5′-dioxo-2,3-dihydrospiro[indene-1,2′-[1,4]ozazolidine]-5-yl)-2′-methylpropanamide;
2-{5-[(azetidin-3-ylmethyl)amino]-3′,5′-dioxo-2,3-dihydrospiro[indene-1,2′-[1,4]ozazolidine]-4′-yl}-N-benzyl-N-[(1S)-1-cyclopropylethyl]acetamide;
N-benzyl-N-[(1S)-1-cyclopropylethyl]-2-{5-[(1,3-dihydroxypropan-2-yl)oxy]-3′,5′-dioxo-2,3-dihydrospiro[indene-1,2′-[1,4]ozazolidine]-4′-yl}acetamide;
1-amino-N-[4′-({[(1S)-1-cyclopropylethyl][(4-fluorophenyl)methyl]carbamoyl}(methyl)-3′,5′-dioxo-2,3-dihydrospiro[indene-1,2′-[1,4]ozazolidine]-5-yl]cyclobutane-1-carboxamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]azetidine-3-carboxamide;
4-{[4′-{benzyl[(1S)-1-cyclopropylethyl]carbamoyl}methyl)-3′,5′-dioxo-2,3-dihydrospiro[indene-1,2′-[1,4]ozazolidine]-5-ylmethyl]amino}benzoic acid;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-2,3-dihydroxypropanamide;
N-benzyl-2-{5-[(methylcarbamoyl)amino]-3′,5′-dioxo-2,3-dihydrospiro[indene-1,2′-[1,4]ozazolidine]-4′-yl}-N-(oxolan-3-yl)acetamide;
N-benzyl-N-[(1S)-1-cyclopropylethyl]-2-{5-[(1,1-dioxo-1λ⁶-thian-4-yl)amino]-3′,5′-dioxo-2,3-dihydrospiro[indene-1,2′-[1,4]ozazolidine]-4′-yl}acetamide;
2-{5-[(2R)-2-amino-2-cyclopropylacetamido]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-(cyclopropylmethyl)-2-{5-[(methylcarbamoyl)amino]-3′,5′-dioxo-2,3-dihydrospiro[indene-1,2′-[1,4]ozazolidine]-4′-yl}-N-[(2-methylphenyl)methyl]acetamide;
N-ethyl-2-{5-[(methylcarbamoyl)amino]-3′,5′-dioxo-2,3-dihydrospiro[indene-1,2′-[1,4]ozazolidine]-4′-yl}-N-[(2-methylphenyl)methyl]acetamide;
N-[(1S)-1-cyclopropylethyl]-2-{3′,5′-dioxo-5-[(pyrrolidin-3-yl)amino]-2,3-dihydrospiro[indene-1,2′-[1,4]ozazolidine]-4′-yl}-N-[(4-fluorophenyl)methyl]acetamide;
2-(5-{[(1-aminocyclopropyl)methyl]amino}-3′,5′-dioxo-2,3-dihydrospiro[indene-1,2′-[1,4]ozazolidine]-4′-yl)-N-[(1S)-1-cyclopropylethyl]-N-[(4-fluorophenyl)methyl]acetamide;
N-benzyl-2-{5-[(methylcarbamoyl)amino]-3′,5′-dioxo-2,3-dihydrospiro[indene-1,2′-[1,4]ozazolidine]-4′-yl}-N-(oxan-4-yl)acetamide;
N-benzyl-N-[(1R)-1-cyclopropyl-2,2,2-trifluoroethyl]-2-{5-[(methylcarbamoyl)amino]-3′,5′-dioxo-2,3-dihydrospiro[indene-1,2′-[1,4]ozazolidine]-4′-yl}acetamide;
N-benzyl-2-{5-[(methylcarbamoyl)amino]-3′,5′-dioxo-2,3-dihydrospiro[indene-1,2′-[1,4]ozazolidine]-4′-yl}-N-(oxan-3-yl)acetamide;
N-[4′-({benzyl[(1S)-1-cyclopropylethyl]carbamoyl}methyl)-3′,5′-dioxo-2,3-dihydrospiro[indene-3,2′-[1,4]ozazolidine]-5-yl]ethanediamide;
2-{5-[(azetidin-3-yl)amino]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-benzyl-N-[(1S)-1-cyclopropylethyl]-2-{3′,5′-dioxo-5-[(2-oxopyrrolidin-3-yl)amino]-2,3-dihydrospiro[indene-1,2′-[1,4]ozazolidine]-4′-yl}acetamide;
2-{[4′-({benzyl[(1S)-1-cyclopropylethyl]carbamoyl}methyl)-3′,5′-dioxo-2,3-dihydrospiro[indene-1,2′-[1,4]ozazolidine]-5-yl]carbamoyl}-2,2-dimethylacetic acid;
N-benzyl-N-[(1S)-1-cyclopropylethyl]-2-{5-[(2-hydroxyethyl)amino]-3′,5′-dioxo-2,3-dihydrospiro[indene-1,2′-[1,4]ozazolidine]-4′-yl}acetamide;
(2R)-2-amino-N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]propanamide;
{[4′-({benzyl[(1S)-1-cyclopropylethyl]carbamoyl}methyl)-3′,5′-dioxo-2,3-dihydrospiro[indene-1,2′-[1,4]ozazolidine]-5-yl]carbamoyl}formic acid;
(2S)-2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-hydroxypropanamide;
N-[(1S)-1-cyclopropylethyl]-2-{3′,5′-dioxo-5-[(piperidin-3-yl)amino]-2,3-dihydrospiro[indene-1,2′-[1,4]ozazolidine]-4′-yl}-N-[(4-fluorophenyl)methyl]acetamide;
N-benzyl-N-[(1S)-1-cyclopropylethyl]-2-{5-[(methylcarbamoyl)amino]-3′,5′-dioxo-2,3-dihydro(3,3-²H₂)spiro[indene-1,2′-[1,4]ozazolidine]-4′-yl}acetamide;
N-[(4-fluorophenyl)methyl]-2-{5-[(methylcarbamoyl)amino]-3′,5′-dioxo-2,3-dihydrospiro[indene-1,2′-[1,4]ozazolidine]-4′-yl}-N-[(2S)-1,1,1-trifluoropropan-2-yl]acetamide;
2-amino-N-[4′-({benzyl[(1R)-1-cyclopropyl-2,2,2-trifluoroethyl]carbamoyl}methyl)-3′,5′-dioxo-2,3-dihydrospiro[indene-1,2′-[1,4]ozazolidine]-5-yl]-2-methylpropanamide;
2-{5-[(methylcarbamoyl)amino]-3′,5′-dioxo-2,3-dihydrospiro[indene-1,2′-[1,4]ozazolidine]-4′-yl}-N-[(2-methylphenyl)methyl]-N-(2-methylpropyl)acetamide;
2-{5-[(4-aminooxolan-3-yl)amino]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-[(4-²H)benzyl]-N-[(1S)-1-cyclopropylethyl]-2-{5-[(methylcarbamoyl)amino]-3′,5′-dioxo-2,3-dihydrospiro[indene-1,2′-[1,4]ozazolidine]-4′-yl}acetamide;
N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-2-methyl-2-(methylamino)propanamide;
(2R)-2-amino-N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-benzyl-N-[(1S)-1-cyclopropylethyl]-2-[(1S)-5-[(methylcarbamoyl)amino]-3′,5′-dioxo-2,3-dihydrospiro[indene-1,2′-[1,4]ozazolidine]-4′-yl]acetamide;
N-benzyl-N-[(1S)-1-cyclopropylethyl]-2-[(1R)-5-[(methylcarbamoyl)amino]-3′,5′-dioxo-2,3-dihydrospiro[indene-1,2′-[1,4]ozazolidine]-4′-yl]acetamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-methylazetidine-3-carboxamide;
N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylazetidine-3-carboxamide;
2-{5-[(2R)-2-amino-2-cyclopropylacetamido]-3′,5′-dioxo-2,3-dihydrospiro[indene-1,2′-[1,4]ozazolidine]-4′-yl}-N-[(4-fluorophenyl)methyl]-N-[(2S)-1,1,1-trifluoropropan-2-yl]acetamide;
N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylazetidine-3-carboxamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-2-(dimethylamino)-2-methylpropanamide;
(2R)-2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-hydroxypropanamide;
2-{5-bromo-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
2-{5-[(3-aminocyclohexyl)amino]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-[1-(azetidin-3-yl)ethyl]-N-benzyl-2-{5-[(methylcarbamoyl)amino]-3′,5′-dioxo-2,3-dihydrospiro[indene-1,2′-[1,4]ozazolidine]-4′-yl}acetamide;
2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-2-methylpropanamide;
N-[(4-fluorophenyl)methyl]-N-[(2S)-1-methoxypropan-2-yl]-2-{5-[(methylcarbamoyl)amino]-3′,5′-dioxo-2,3-dihydrospiro[indene-1,2′-[1,4]ozazolidine]-4′-yl}acetamide;
3-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]oxetane-3-carboxamide;
2-amino-N-[(1S)-4′-({[(4-bromophenyl)methyl][(1S)-1-cyclopropylethyl]carbamoyl}methyl)-3′,5′-dioxo-2,3-dihydrospiro[indene-1,2′-[1,4]ozazolidine]-5-yl]-2-methylpropanamide;
2-amino-N-[(1R)-4′-({[(4-bromophenyl)methyl][(1S)-1-cyclopropylethyl]carbamoyl}methyl)-3′,5′-dioxo-2,3-dihydrospiro[indene-1,2′-[1,4]ozazolidine]-5-yl]-2-methylpropanamide;
2-{5-[(2R)-2-amino-2-(oxetan-3-yl)acetamido]-3′,5′-dioxo-2,3-dihydrospiro[indene-1,2′-[1,4]ozazolidine]-4′-yl}-N-benzyl-N-[(1S)-1-cyclopropylethyl]acetamide;
1-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3-difluorocyclobutane-1-carboxamide;
2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1S)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1R)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]-N-[(2S)-1,1,1-trifluoropropan-2-yl]acetamide;
N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]-N-[(2S)-1,1,1-trifluoropropan-2-yl]acetamide;
N-[(1S)-1-cyclopropylethyl]-N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]acetamide;
N-[(1S)-1-cyclopropylethyl]-N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]acetamide;
(2S)-2-amino-N-[(1S)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1S)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2S)-2-amino-N-[(1R)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1R)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
N-benzyl-N-[(1R)-1-cyclopropyl-2,2,2-trifluoroethyl]-2-[(1S)-5-[(methylcarbamoyl)amino]-3′,5′-dioxo-2,3-dihydrospiro[indene-1,2′-[1,4]ozazolidine]-4′-yl]acetamide;
N-benzyl-N-[(1R)-1-cyclopropyl-2,2,2-trifluoroethyl]-2-[(1R)-5-[(methylcarbamoyl)amino]-3′,5′-dioxo-2,3-dihydrospiro[indene-1,2′-[1,4]ozazolidine]-4′-yl]acetamide;
(2R)-2-amino-N-[(1S)-4′-({[4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1R)-4′-({[(4-fluorophenyl)methy 1][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1S)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1R)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(1R)-1-cyclopropyl-2,2,2-trifluoroethyl]-N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]acetamide;
N-[(1R)-1-cyclopropyl-2,2,2-trifluoroethyl]-N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]acetamide;
N-[(1S)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
N-[(1R)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
(2R)-2-amino-N-[4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(1S)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
N-[(1R)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
(2S)-2-amino-N-[4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
(2S)-2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]azetidine-3-carboxamide;
N-benzyl-N-[1-(1-methylazetidin-3-yl)ethyl]-2-{5-[(methylcarbamoyl)amino]-3′,5′-dioxo-2,3-dihydrospiro[indene-1,2′-[1,4]ozazolidine]-4′-yl}acetamide;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1R)-3′-(2-{[(1R)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-benzyl-2-[(1R)-5-bromo-2′,4′-dioxo-2,3-dihydro-3′H-spiro[indene-1,5′-[1,3]ozazolidin]-3′-yl]-N-[(1S)-1-cyclopropylethyl]acetamide;
2-[(1S)-5-amino-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-[(1R)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-[(1S)-3′-(2-{[(1R)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-[(1R)-1-cyclopropylethyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1R)-1-cyclopropylethyl]-N-(4-fluorobenzyl)-2-{(1S)-5-[(methylcarbamoyl)amino]-2′,4′-dioxo-2,3-dihydro-3′H-spiro[indene-1,5′-[1,3]ozazolidin]-3′-yl}acetamide;
N-[(1R)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-[(1S)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]azetidine-1-carboxylate;
2-[(1R)-5-(6-aminopyridin-3-yl)-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-{(1R)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-[(1S)-1-cyclopropylethyl]-N-(4-fluorobenzyl)acetamide;
N-[(1S)-1-cyclopropylethyl]-2-[(1R)-2′,4′-dioxo-5-(pyridin-3-yl)-2,3-dihydro-3′H-spiro[indene-1,5′-[1,3]ozazolidin]-3′-yl]-N-(4-fluorobenzyl)acetamide;
N-benzyl-2-(7-bromo-2′,4′-dioxo-2,3-dihydro-3′H-spiro[chromene-4,5′-[1,3]ozazolidin]-3′-yl)-N-[(1S)-1-cyclopropylethyl]acetamide;
2-[(1R)-5-(azetidin-3-yl)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
2-[(1R)-5-(1-acetylazetidin-3-yl)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-benzyl-N-[(1S)-1-cyclopropylethyl]-2-[(1R)-5-(1-methylazetidin-3-yl)-2′,4′-dioxo-2,3-dihydro-3′H-spiro[indene-1,5′-[1,3]ozazolidin]-3′-yl]acetamide;
3-[(1R)-3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]-N-methylazetidine-1-carboxamide;
N-benzyl-2-{(1R)-5-[1-(cyclopropylcarbonyl)azetidin-3-yl]-2′,4′-dioxo-2,3-dihydro-3′H-spiro[indene-1,5′-[1,3]ozazolidin]-3′-yl}-N-[(1S)-1-cyclopropylethyl]acetamide;
N-benzyl-N-[(1S)-1-cyclopropylethyl]-2-{(1R)-5-[1-(2-hydroxy-2-methylpropanoyl)azetidin-3-yl]-2′,4′-dioxo-2,3-dihydro-3′H-spiro[indene-1,5′-[1,3]ozazolidin]-3′-yl}acetamide;
N-benzyl-N-[(1S)-1-cyclopropylethyl]-2-{(1R)-5-[1-(methylsulfonyl)azetidin-3-yl]-2′,4′-dioxo-2,3-dihydro-3′H-spiro[indene-1,5′-[1,3]ozazolidin]-3′-yl}acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]cyclobutanecarboxamide;
3-ethoxy-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-5-oxo-D-prolinamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-5-oxo-L-prolinamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanimide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]cyclopropanecarboxamide;
N-(4-fluorobenzyl)-2-{(1R)-5-[(methoxyacetyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(1R)-5-[(ethoxyacetyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-3-methylbutanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylpropanamide;
2-[(1R)-5-(acetylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(7-amino-2′,4′-dioxo-2,3-dihydro-3′H-spiro[chromene-4,5′-[1,3]ozazolidin]-3′-yl)-N-benzyl-N-[(1S)-1-cyclopropylethyl]acetamide;
N-(4-fluorobenzyl)-2-[(1R)-5-{[(2-methoxyethyl)(methyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[(dimethylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-{[methyl(2-methylpropyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(cyclopropylmethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(cyclobutylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-({[(2R)-1-hydroxy-3-methylbutan-2-yl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-({[(2S)-tetrahydrofuran-2-ylmethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-({[(2R)-tetrahydrofuran-2-ylmethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-({[2-(propan-2-yloxy)ethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(cyclopropylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-{[ethyl(methyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(diethylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyrrolidine-1-carboxamide;
2-[(1R)-5-({[2-(dimethylamino)ethyl](methyl)carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N²-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-L-leucinamide;
N-(4-fluorobenzyl)-2-[(1R)-5-{[(2-hydroxy ethyl)(propyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(2-methoxyethyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(3-hydroxypropyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-({[(2S)-1-hydroxy-3-methylbutan-2-yl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[methyl(propan-2-yl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(2-cyanoethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2′-yl]acetamide;
N-(4-fluorobenzyl)-2-[(1R)-5-{[methyl(propyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-benzyl-N-[(1S)-1-cyclopropylethyl]-2-{7′-[(methylcarbamoyl)amino]-2′,4′-dioxo-2,3-dihydro-3′H-spiro[chromene-4,5′-[1,3]ozazolidin]-3′-yl}acetamide;
2-[7-(acetylamino)-2′,4′-dioxo-2,3-dihydro-3′H-spiro[chromene-4,5′-[1,3]ozazolidin]-3′-yl]-N-benzyl-N-[(1S)-1-cyclopropylethyl]acetamide;
N-[(1S)-1-cyclopropylethyl]-2-[(1R)-2′,4′-dioxo-5-(pyridin-4-yl)-2,3-dihydro-3′H-spiro[indene-1,5′-[1,3]ozazolidin]-3′-yl]-N-(4-fluorobenzyl)acetamide;
N-[(1S)-1-cyclopropylethyl]-N-(4-fluorobenzyl)-2-[(1R)-5-(6-hydroxypyridazin-3-yl)-2′,4′-dioxo-2,3-dihydro-3′H-spiro[indene-1,5′-[1,3]ozazolidin]-3′-yl]acetamide;
N-[(1S)-1-cyclopropylethyl]-2-[(1R)-2′,4′-dioxo-5-(1H-pyrazol-4-yl)-2,3-dihydro-3′H-spiro[indene-1,5′-[1,3]ozazolidin]-3′-yl]-N-(4-fluorobenzyl)acetamide;
N-benzyl-N-[(1S)-1-cyclopropylethyl]-2-[2′,4′-dioxo-7-(6-oxo-1,6-dihydropyridin-3-yl)-2,3-dihydro-3′H-spiro[chromene-4,5′-[1,3]ozazolidin]-3′-yl]acetamide;
2-{(1R)-2′,4′-dioxo-5-[(tetrahydrofuran-2-ylmethyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[(cyclopentylmethyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[(2,2-dimethylbutyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-2′,4′-dioxo-5-[(tetrahydrofuran-3-ylmethyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-{(1R)-5-[(2-methylpropyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
tert-butyl [(2R)-1-{[3′-(2-{benzyl[(1S)-1-cyclopropylethyl]amino}-2-oxoethyl)-2′,4′-dioxo-2,3-dihydrospiro[chromene-4,5′-[1,3]ozazolidin]-7-yl]amino}-3-methyl-1-oxobutan-2-yl]carbamate;
N-[3′-(2-{benzyl[(1S)-1-cyclopropylethyl]amino}-2-oxoethyl)-2′,4′-dioxo-2,3-dihydrospiro[chromene-4,5′-[1,3]ozazolidin]-7-yl]-D-valinamide;
N-[(1S)-1-cyclopropylethyl]-2-[(1R)-2′,4′-dioxo-5-(pyridin-2-yl)-2,3-dihydro-3′H-spiro[indene-1,5′-[1,3]ozazolidin]-3′-yl]-N-(4-fluorobenzyl)acetamide;
N-[(1S)-1-cyclopropylethyl]-N-(3-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropyl-2,2,2-trifluoroethyl]-N-(4-fluorobenzyl)-2-{7′-[(methylcarbamoyl)amino]-2,4-dioxo-2′,3′-dihydro-3′H-spiro[1,3-ozazolidine-5,4′-thiochromen]-3-yl}acetamide;
2-[(1R)-5-(carbamoylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1S)-1-cyclopropyl-2,2,2-trifluoroethyl]-N-(4-fluorobenzyl)-2-{7′-[(methylcarbamoyl)amino]-1′,1′-dioxido-2,4-dioxo-2′,3′-dihydro-3′H-spiro[1,3-ozazolidine-5,4′-thiochromen]-3-yl}acetamide;
N-(4-fluorobenzyl)-2-((R)-5-(3-(N-methylsulfamoyl)ureido)-2′,4′-dioxo-2,3-dihydrospiro[indene-1,5′-ozazolidine]-3′-yl)-N—((S)-1,1,1-trifluoropropan-2-yl)acetamide
methyl {[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}sulfamate;
2-((R)-5-(3-(N-(cyclopropylmethyl)sulfamoyl)ureido)-2′,4′-dioxo-2,3-dihydrospiro[indene-1,5′-ozazolidine]-3′-yl)-N-(4-fluorobenzyl)-N—((S)-1,1,1-trifluoropropan-2-yl)acetamide
(1R)-3′-(2-{benzyl[(1S)-1-cyclopropylethyl]amino}-2-oxoethyl)-N-(2-methylpropyl)-2′,4′-dioxo-2,3-dihydrospiro[indene-1,5′-[1,3]ozazolidine]-5-carboxamide;
N-[(1S)-1-cyclopropylethyl]-N-(2-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,3-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-chloro-4-fluorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3-chloro-4-fluorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-[4-(trifluoromethyl)benzyl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1-methyl-1H-pyrazole-4-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1-methyl-1H-pyrazole-3-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-4-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-2-carboxamide;
2-(5-bromo-4-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
2-(5-bromo-6-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
(1R)-3′-(2-{benzyl[(1S)-1-cyclopropylethyl]amino}-2-oxoethyl)-N-[(2R)-1-hydroxy-3-methylbutan-2-yl]-2′,4′-dioxo-2,3-dihydrospiro[indene-1,5′-[1,3]ozazolidine]-5-carboxamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-[(5-methyl-1,3,4-thiadiazol-2-yl)methyl]acetamide;
2-(5-amino-4-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{4-fluoro-5-[(methylcarbamoyl)amino]-2′,4′-dioxo-2,3-dihydro-3′H-spiro[indene-1,5′-[1,3]ozazolidin]-3′-yl}acetamide;
N-(4-cyanobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3-methylbenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,4-dichlorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-methoxybenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3-methoxybenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-methylbenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-[(1R)-5-{[(1-methyl-1H-pyrazol-4-yl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{7′-[1-(2-amino-2-oxoethyl)-1′H-pyrazol-4-yl]-2′,4′-dioxo-2,3-dihydro-3′H-spiro[chromene-4,5′-[1,3]ozazolidin]-3′-yl}-N-benzyl-N-[(1S)-1-cyclopropylethyl]acetamide;
2-{(1R)-2′,4′-dioxo-5-[(phenylcarbamoyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-2′,4′-dioxo-5-[(pyridin-3-ylcarbamoyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-(5-amino-6-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{6-fluoro-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetamide;
N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(dimethylamino)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(4-methylpyrimidin-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[5-(difluoromethyl)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(6-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1R)-5-[1-(2-methylpropyl)-1H-pyrazol-4-yl]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(1R)-5-[4-(difluoromethyl)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(3-fluoropyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(1,3-thiazol-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-N-methylbenzamide;
2-{(1R)-5-[2-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(5-cyanopyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(1-methyl-1H-1,2,4-triazol-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(5-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-(6-cyanopyridin-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(5-fluoro-3-methylpyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(5-methyl-1,3,4-thiadiazol-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(4-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(6-methoxypyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[2-(dimethylamino)pyrimidin-5-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(acetylamino)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(1-methyl-1H-1,2,4-triazol-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-cyclopropyl-5-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]furan-2-carboxamide;
N-(4-fluorobenzyl)-2-[(1R)-5-(5-fluoropyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
4-fluoro-3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
5-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-N-methylpyridine-2-carboxamide;
6-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-3-carboxamide;
N-(4-fluorobenzyl)-2-[(1R)-5-(2-methoxypyrimidin-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
6-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-2-carboxamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]thiophene-3-carboxamide;
2-{(1R)-5-[4-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(1,3-dimethyl-2,4-dioxo-1,2,3,4-tetrahydropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4-cyanopyridin-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-4-carboxamide;
2-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
2-{(1R)-5-[3-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(6-cyano-5-methoxypyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(1-methyl-1H-pyrazol-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(1,3,5-trimethyl-1H-pyrazol-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(3,4-difluorophenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2,5-difluoro-4-methoxyphenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyrimidin-5-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-2′,4′-dioxo-5-[5-(trifluoromethyl)pyridin-3-yl]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-2′,4′-dioxo-5-(1,3-thiazol-2-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2-cyanopyridin-4-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyrazin-2-yl)-2,3-dihydro-3′H-spiro[idene-1,5′-[1,3]ozazolidin]-3′-yl]-N-(4-fluorobenzyl)-N-[(2S)-1,1,1-trifluoropropan-2-yl]acetamide;
2-{(1R)-5-[4-(carbamoylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(2-cyanopyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(6-cyano-5-fluoropyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
2-[(1R)-5-{[(4-aminophenyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-{6-[(methylcarbamoyl)amino]-2′,4′-dioxo-3,4-dihydro-2H,3′H-spiro[naphthalene-1,5′-[1,3]ozazolidin]-3′-yl}-N-[(2S)-1,1,1-trifluoropropan-2-yl]acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(pyrimidin-5-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(pyrimidin-2-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-thiazol-5-ylmethyl)acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]prolinamide;
N²-(4-fluorobenzyl)-N²-({(1R)-5-[(methylcarbamoyl)amino]-2′,4′-dioxo-2,3-dihydro-3′H-spiro[indene-1,5′-[1,3]ozazolidin]-3′-yl}acetyl)-L-alaninamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1H-pyrazole-5-carboxamide;
N-(4-fluorobenzyl)-2-[(1R)-5-{[(methylsulfamoyl)acetyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(methylsulfonyl)acetyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(dimethylsulfamoyl)acetyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-2′,4′-dioxo-5-[(sulfamoylacetyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidine-1-carboxamide
N-benzyl-N-ethyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
methyl N′-cyano-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamimidothioate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanamide;
N-(4-fluorobenzyl)-2-{(1R)-5-[(methylcarbamothioyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(tetrabydro-2H-pyran-4-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-oxazol-4-ylmethyl)acetamide;
2-[(1R)-5-(5-cyanothiophen-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4-cyano-3-fluorophenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(5-cyanothiophen-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(N″-cyano-N′-methylcarbamimidamido)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4,6-difluoropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[2-(acetylamino)-5-methylpyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(5-fluoropyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(furan-3-yl)-2′,4′-dioxo-2,3-dihydro-3′H-spiro[indene-1,5′-[1,3]ozazolidin]-3′-yl]-N-[(2S)-1,1, l-trifluoropropan-2-yl]acetamide;
2-[(1R)-2′,4′-dioxo-5-(2-oxo-2,3-dihydro-1H-benzimidazol-5-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(4-methylpyrimidin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-2′,4′-dioxo-5-[1-(tetrahydro-2H-pyran-2-yl)-1H-pyrazol-3-yl]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(5-fluoro-6-methylpyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[6-(difluoromethyl)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(difluoromethyl)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(furan-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[1-(ethoxymethyl)-1H-imidazol-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(5-cyano-1,2-dimethyl-6-oxo-1,6-dihydropyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]thiophene-2-carboxamide;
2-[(1R)-5-(2,6-dioxo-1,2,5,6-tetrahydropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2,4-dimethyl-1,3-thiazol-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[1-(cyanomethyl)-3,5-dimethyl-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[3-(carbamoylimino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-[3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)-2,2-dimethylpropyl]propanamide;
(1R)-3′-(2-{benzyl[(1S)-1-cyclopropylethyl]amino}-2-oxoethyl)-N-methyl-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(propan-2-yl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(2-methoxyethyl)-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(tetrahydrofuran-2-ylmethyl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(tetrahydrofuran-3-ylmethyl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(cyanomethyl)-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl) -N-(3-hydroxypropyl)-2′,4′-dioxo-2,3-dihydrospiro[indene-1,5′-[1,3]ozazolidine]-5-carboxamide;
3′-{2-[benzyl(1-cyclopropylethyl)amino]-2-oxoethyl}-N-(1-hydroxy-3-methylbutan-2-yl)-2′,4′-dioxo-2,3-dihydrospiro[indene-1,5′-[1,3]ozazolidine]-5-carboxamide;
N-(1-amino-4-methyl-1-oxopentan-2-yl)-3′-{2-[benzyl(1-cyclopropylethyl)amino]-2-oxoethyl}-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-[2-(diethylamino)ethyl]-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-[3-(1H-imidazol-1-yl)propyl]-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-[3-(2-oxopyrrolidin-1-yl)propyl]-2,3-dihydrospiro[indene-1,5′-[1,3]ozazolidine]-5-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]tetrahydropyrimidine-1(2H)-carboxamide;
3-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]tetrahydropyrimidine-1(2H)-carboxamide;
N-(4-fluorobenzyl)-2-{(1R)-5-[1-(2-hydroxyethyl)-1H-pyrazol-4-yl]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-phenylacetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N,N-dibenzyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
(2R)-3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-hydroxy-2-methylpropanamide;
(2S)-3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-hydroxy-2-methylpropanamide;
2-[(1R)-2′,4′-dioxo-5-({[3-(pyrrolidin-1-ylmethyl)phenyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-({[3-(morpholin-4-ylmethyl)phenyl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N³-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-N-methyl-beta-alaninamide;
N-[3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)phenyl]propanamide;
2-(6-bromo-2′,4,4′-trioxo-3,4-dihydro-2H,3′H-spiro[naphthalene-1,5′-[1,3]ozazolidin]-3′-yl)-N-[(1S)-1-cyclopropyl-2,2,2-trifluoroethyl]-N-(4-fluorobenzyl)acetamide;
N-(2-chlorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)acetamide;
N-[(1S)-1-cyclopropyl-2,2,2-trifluoroethyl]-N-(4-fluorobenzyl)-2-{6-[(methylcarbamoyl)amino]-2′,4,4′-trioxo-3,4-dihydro-2H,3′H-spiro[naphthalene-1,5′-[1,3]ozazolidin]-3′-yl}acetamide;
N-(4-fluorobenzyl)-2-{(1S)-6-[(methylcarbamoyl)amino]-2′,4′-dioxo-3,4-dihydro-2H,3′H-spiro[naphthalene-1,5′-[1,3]ozazolidin]-3′-yl}-N-[(2S)-1,1,1-trifluoropropan-2-yl]acetamide;
N-(4-fluorobenzyl)-2-{(1R)-6-[(methylcarbamoyl)amino]-2′,4′-dioxo-3,4-dihydro-2H,3′H-spiro[naphthalene-1,5′-[1,3]ozazolidin]-3′-yl}-N-[(2S)-1,1,1-trifluoropropan-2-yl]acetamide;
2-[(1R)-5-(4,5-dihydro-1H-imidazol-2-ylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(6-amino-2′,4′-dioxo-3,4-dihydro-2H,3′H-spiro[naphthalene-1,5′-[1,3]ozazolidin]-3′-yl)-N-[(1S)-1-cyclopropyl-2,2,2-trifluoroethyl]-N-(4-fluorobenzyl)acetamide;
2-[(1R)-5-({[3-(acetylamino)propyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-({[2-(acetylamino)ethyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
tert-butyl [3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)-2,2-dimethylpropyl]carbamate;
N-[2-({[(1R)-3′-(2-{(4-fluorobenzyl)-N-[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)ethyl]propanamide;
2-[(1R)-5-{[(3-amino-2,2-dimethylpropyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-({[3-(acetylamino)phenyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
methyl 2-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]amino}-4,5-dihydro-1H-imidazole-1-carboxylate;
2-{5-bromo-6-[(methylcarbamoyl)amino]-2′,4′-dioxo-3,4-dihydro-2H,3′H-spiro[naphthalene-1,5′-[1,3]ozazolidin]-3′-yl}-N-[(1S)-1-cyclopropyl-2,2,2-trifluoroethyl]-N-(4-fluorobenzyl)acetamide;
N-cyclohexyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-cyclopentyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropyl-2,2,2-trifluoroethyl]-N-(4-fluorobenzyl)-2-{4-hydroxy-6-[(methylcarbamoyl)amino]-2′,4′-dioxo-3,4-dihydro-2H,3′H-spiro[naphthalene-1,5′-[1,3]ozazolidin]-3′-yl}acetamide;
N-benzyl-N-[1-(furan-2-yl)ethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropyl-2,2,2-trifluoroethyl]-N-(4-fluorobenzyl)-2-{4-fluoro-6-[(methylcarbamoyl)amino]-2′,4′-dioxo-3,4-dihydro-2H,3′H-spiro[naphthalene-1,5′-[1,3]ozazolidin]-3′-yl}acetamide;
N-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-beta-alanine;
N-cyclopropyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl N-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-beta-alaninate;
N-(3,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(4-fluorobenzyl)-N-(3-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(1S)-5-amino-2′,4′-dioxo-2,3-dihydro-3′H-spiro[indene-1,5′-[1,3]ozazolidin]-3′-yl]-N-[(1R)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
ethyl ({(3R)-3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(3R)-piperidin-3-yl]acetamide;
ethyl ({(3R)-3-[{[(1R)-5-bromo-2′,4′-dioxo-2,3-dihydro-3′H-spiro[indene-1,5′-[1,3]ozazolidin]-3-yl]acetyl}(4-fluorobenzyl)amino]piperidin-1-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(methylsulfonyl)piperidin-4-yl]acetamide;
N-(4-fluorobenzyl)-2-{6-fluoro-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-(5-amino-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3-yl}-N-(1-phenylethyl)acetamide;
2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)-N-[2-(trifluoromethyl)benzyl]acetamide;
2-(5-bromo-6-fluoro-2′,4′-dioxo-2,3-dihydro-3′H-spiro[indene-1,5′-[1,3]ozazolidin]-3′-yl) -N-(4-fluorobenzyl)-N-[(2S)-1,1,1-trifluoropropan-2-yl]acetamide;
N-benzyl-N-(cyanomethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-(2-cyanoethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(methylsulfonyl)propan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(1-methyl-1H-pyrazol-4-yl)methyl]acetamide;
N-[(1S)-1-cyclopropylethyl]-N-(4-fluorobenzyl)-2-{4-methoxy-5-[(methylcarbamoyl)amino]-2′,4′-dioxo-2,3-dihydro-3′H-spiro[indene-1,5′-[1,3]ozazolidin]-3′-yl}acetamide;
2-[5-(acetylamino)-4-methoxy-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-[(1R)-5-({[3-(acetylamino)-2,2-dimethylpropyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclopentyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(pyridin-3-yl)acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1-methyl-1H-pyrazol-4-yl)acetamide;
N-benzyl-N-(2,2-difluorocyclopentyl)-2-{5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-(2-methylcyclopropyl)acetamide;
N-(2,2-dimethylcyclopentyl)-N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-(2-methylcyclopentyl)acetamide;
2-(5-{[(2-cyanoethyl)carbamoyl]amino}-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
methyl [(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamate;
4-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)butanoic acid;
N²-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylalaninamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylalaninamide;
N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
N-cyclopentyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-oxazol-4-ylmethyl)acetamide;
N-benzyl-2-{(1R)-5-[(methylcarbamoyl)aminol]-2′,4′-dioxo-2,3-dihydro-3′H-spiro[indene-1,5′-[1,3]ozazolidin]-3′-yl}-N-[1-(pyridin-2-yl)ethyl]acetamide;
N-(4-chlorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3,5-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-(5-{[(cyanomethyl)carbamoyl]amino}-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
2-[(1R)-5-{[(cyanomethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclohexyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-pyrrolidin-3-yl]acetamide;
N-(4-fluorobenzyl)-N-(trans-3-hydroxycyclobutyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-1-(methylsulfonyl)pyrrolidin-3-yl]acetamide;
ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidin-1-yl}sulfonyl)carbamate;
N-(1-cyanopropan-2-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
5-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)pentanoic acid;
N-[(1S)-1-cyclopropyl-2,2,2-trifluoroethyl]-N-(4-fluorobenzyl)-2-[4-fluoro-5-(1-methyl-1H-pyrazol-4-yl)-2′,4′-dioxo-2,3-dihydro-3′H-spiro[indene-1,5′-[1,3]ozazolidin]-3′-yl]acetamide;
N-(4-fluorobenzyl)-2-[6-fluoro-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-[(3R)-1-acetylpyrrolidin-3-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-1-(2-sulfamoylethyl)pyrrolidin-3-yl]acetamide;
N-(4-fluorobenzyl)-N-(trans-4-hydroxycyclohexyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
methyl (1R,3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-3-(methylsulfonyl)tetrahydropyrimidine-1(2H)-carboxamide;
N²-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]glycinamide;
2-{(1R)-5-[(methylcarbamoyl)amino]-2′,4′-dioxo-2,3-dihydro-3′H-spiro[indene-1,5′-[1,3]ozazolidin]-3′-yl}-N-[1-(2-methylphenyl)ethyl]-N-(2-methylpropyl)acetamide;
N-(2-chlorobenzyl)-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)acetamide;
N-(4-fluorobenzyl)-N-(4-methoxyphenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(4-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(2-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(trifluoromethyl)cyclopropyl]acetamide;
2-[(1R)-5-(3,4-dihydro-2H-pyrrol-5-ylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(3,4-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(2,5-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3-chlorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{5-[1-(2-amino-2-oxoethyl)-1H-pyrazol-4-yl]-6-fluoro-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(3R)-pyrrolidin-3-yl]acetamide;
ethyl ({(3R)-3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]pyrrolidin-1-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(piperidin-4-yl)acetamide;
ethyl ({4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]azetidine-1-carboxylate;
N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}-N-[2-(trifluoromethyl)benzyl]acetamide;
N-(2-chloro-4-fluorobenzyl)-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(3-methoxyphenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-(pyridin-4-yl)acetamide;
N-(4-fluorobenzyl)-2-(6-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl)-N-[(2S)-1,1,1-trifluoropropan-2-yl]acetamide;
methyl (1S,3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(tetrahydro-2H-pyran-4-yl)acetamide;
N-(1,1-dioxidotetrahydro-2H-thiopyran-4-yl)-N-(4-fluorobenzyl)-2-{(1R)-5-[(methylcarbonyl)amino]-2′,4′-dioxo-2,3-dihydro-3′H-spiro[indene-1,5′-[1,3]ozazolidin]-3′-yl}acetamide;
N-(2,2-difluorocyclopentyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-cyclohexyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-[(1S)-1-cyclopropyl-2,2,2-trifluoroethyl]-N-(4-fluorobenzyl)-2-[7-(1-methyl-1H-pyrazol-4-yl)-2′,4′-dioxo-1H,3′H-spiro[isochromene-4,5′-[1,3]ozazolidin]-3′-yl]acetamide;
2-(7-amino-2′,4′-dioxo-1H,3′H-spiro[isochromene-4,5′-[1,3]ozazolidin]-3′-yl)-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{7′-[(methylcarbamoyl)amino]-2′,4′-dioxo-1H,3′H-spiro[isochromene-4,5′-[1,3]ozazolidin]-3′-yl}acetamide;
N-(4-fluorobenzyl)-2-{7′-[(methylcarbamoyl)amino]-2′,4′-dioxo-1H,3′H-spiro[isochromene-4,5′-[1,3]ozazolidin]-3′-yl}-N-[(2S)-1,1,1-trifluoropropan-2-yl]acetamide;
N-(4-fluorobenzyl)-2-[7-(1-methyl-1H-pyrazol-4-yl)-2′,4′-dioxo-1H,3′H-spiro[isochromene-4,5′-[1,3]ozazolidin]-3′-yl]-N-[(2S)-1,1,1-trifluoropropan-2-yl]acetamide;
N-(4-fluorobenzyl)-2-(7-{1-[2-(methylamino)-2-oxoethyl]-1H-pyrazol-4-yl}-2′,4′-dioxo-1H,3′H-spiro[isochromene-4,5′-[1,3]ozazolidin]-3′-yl)-N-[(2S)-1,1,1-trifluoropropan-2-yl]acetamide;
N-(4-fluorobenzyl)-N-{1-[2-(methylamino)-2-oxoethyl]-1H-pyrazol-4-yl}-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[1-(cyanomethyl)-1H-pyrazol-4-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl 3-{1-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]ethyl}azetidine-1-carboxylate;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylic acid;
ethyl ({(3S)-3-[(4-fluorobenzyl){[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
ethyl ({(3S)-3-[{[(1R)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}(4-fluorobenzyl)amino]pyrrolidin-1-yl}sulfonyl)carbamate;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}acetate;
ethyl ({(3R)-3-[(4-fluorobenzyl){[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]piperidin-1-yl}sulfonyl)carbamate;
ethyl ({(3R)-3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]piperidin-1-yl}sulfonyl)carbamate;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}acetic acid;
N-(4-fluorobenzyl)-2-{(4S)-7-[(methylcarbamoyl)amino]-2′,4′-dioxo-1H,3′H-spiro[isochromene-4,5′-[1,3]ozazolidin]-3′-yl}-N-[(2S)-1,1,1-trifluoropropan-2-yl]acetamide;
N-(4-fluorobenzyl)-2-{(4R)-7-[(methylcarbamoyl)amino]-2′,4′-dioxo-1H,3′H-spiro[isochromene-4,5′-[1,3]ozazolidin]-3′-yl}-N-[(2S)-1,1,1-trifluoropropan-2-yl]acetamide;
tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
tert-butyl (3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-piperidin-3-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3S)-piperidin-3-yl]acetamide;
N-[4-amino-3-(hydroxymethyl)butan-2-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({(3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzamide;
tert-butyl 2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(pyrrolidin-2-ylmethyl)acetamide;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexylidene}acetate;
N-ethyl-4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxamide;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexylidene}acetic acid;
N-(4-fluorobenzyl)-2-[(1R)-5-(oxetan-3-ylamino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzamide;
N-[(1-acetylpyrrolidin-2-yl)methyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{[1-(methylsulfonyl)pyrrolidin-2-yl]methyl}acetamide;
2-{[(4-fluorobenzyl) ({(1R)-5-[(methylcarbamoyl) amino]-2′,4′-dioxo-2,3-dihydro-3′H-spiro[indene-1,5′-[1,3]ozazolidin]-3′-yl}acetyl)amino]methyl}-N-methylpyrrolidine-1-carboxamide;
N-(3,4-difluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{[1-(methylsulfamoyl)pyrrolidin-2-yl]methyl}acetamide;
N-(cyclopropylmethyl)-N-(2,3-dihydro-1H-inden-1-yl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexyl}acetic acid;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-3-methylazetidin-1-yl}sulfonyl)carbamate;
N-(3-cyanophenyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
tert-butyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}carbamate;
N-(3-aminocyclobutyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[3-(acetylamino)cyclobutyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{3-[(methylsulfonyl)amino]cyclobutyl}acetamide;
2,2′-({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}imino)diacetic acid;
N-{3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}glycine;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxylate;
methyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}carbamate;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}sulfamoyl)carbamate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxylic acid;
ethyl ({4-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]piperidin-1-yl}sulfonyl)carbamate;
2-[(1S,3S)-5-bromo-3-hydroxy-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(5-chloro-4-cyano-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
benzyl 3-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}azetidine-1-carboxylate;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoate;
N-(4-fluorobenzyl)-2-[(1S,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
ethyl ({3,3-difluoro-4-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
ethyl ({3,3-difluoro-4-[(4-fluorobenzyl){[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
N-(4-fluorobenzyl)-2-[(1S)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1S)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-N-(3-methoxycyclobutyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-N-methylcyclobutanecarboxamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxamide;
2-(5-amino-4-cyano-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetic acid;
(2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidin-1-yl)acetic acid;
methyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetate;
{3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetic acid;
tert-butyl {4-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]cyclohexylidene}acetate;
tert-butyl {4-[(4-fluorobenzyl){[(1S)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetate;
{4-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetic acid;
N-(4-fluorobenzyl)-2-[(1S,3R)-3-fluoro-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(azetidin-3-ylmethyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl {trans-4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexyl}carbamate;
N-(trans-4-aminocyclohexyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{trans-4-[(methylsulfonyl)amino]cyclohexyl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(trans-4-{[(trifluoromethyl)sulfonyl]amino}cyclohexyl)acetamide;
tert-butyl 6-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-2-azaspiro[3.3]heptane-2-carboxylate;
N-(2-azaspiro[3.3]hept-6-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(1S,3R)-5-bromo-3-hydroxy-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1-acetylazetidin-3-ylmethyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-[(2S)-1,1,1-trifluoropropan-2-yl]-2-(2′,3,4′-trioxo-1,2,3,4,8,9-hexahydro-3′H-spiro[cyclopenta[f]quinazoline-7,5′-[1,3]ozazolidin]-3′-yl)acetamide;
ethyl [(3-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}azetidin-1-yl)sulfonyl]carbamate;
ethyl [(2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidin-1-yl)sulfonyl]carbamate;
ethyl ({6-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-2-azaspiro[3.3]hept-2-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(3-{[(trifluoromethyl)sulfonyl]amino}cyclobutyl)acetamide;
tert-butyl 3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]benzoate;
tert-butyl 3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]benzoate;
3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]benzoic acid;
N-(4-fluorobenzyl)-2-[(1S)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(azetidin-3-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]azetidin-1-yl}sulfonyl)carbamate;
2-[(1S)-5-bromo-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1S,3R)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-[(1S)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-D-valinamide;
N-benzyl-N-[(1S)-1-cyclopropylethyl]-2-[2′,4′-dioxo-5-(2H-tetrazol-5-yl)-2,3-dihydro-3′H-spiro[indene-1,5′-[1,3]ozazolidin]-3′-yl]acetamide;
2,2′-({[3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]carbonyl}imino)diacetic acid;
N-(4-fluorobenzyl)-2-[(1S,3R)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1S,3R)-5-[1-(2-amino-2-oxoethyl)-1H-pyrazol-4-yl]-3-hydroxy-2′,4′-dioxo-2,3-dihydro-3′H-spiro[indene-1,5′-[1,3]ozazolidin]-3′-yl}-N-[(4-fluorobenzyl)-N-(2S)-1,1,1-trifluoropropan-2-yl]acetamide;
N-(4-fluorobenzyl)-2-[(1S)-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
methyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanoate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanoic acid;
2-{(1S)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R,3R)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R,3R)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1S,3S)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1S)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S,3R)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyridin-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(pyridin-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(pyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(6-oxo-1,6-dihydropyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(6-oxo-1,6-dihydropyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-phenyl-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-phenyl-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclohexyl-N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-(tetrahydro-2H-pyran-4-yl)acetamide;
3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclobutanecarboxylic acid;
4-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexanecarboxylic acid;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-{trans-4-[(methylsulfonyl)amino]cyclohexyl}acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-(3-{[(trifluoromethyl)sulfonyl]amino}cyclobutyl)acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-[1-(methylsulfonyl)piperidin-4-yl]acetamide;
2-[(1R,3S)-5-bromo-3-hydroxy-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R,3S)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2,2′-({3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclobutyl}imino)diacetic acid;
N-(4-fluorobenzyl)-2-[(1S,3R)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(1,2-oxazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1S)-5-(1,2-oxazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1S,3R)-3-fluoro-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-[(1R,3S)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R,3S)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
{4-[(4-fluorobenzyl){[(1S,3R)-3-fluoro-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetic acid;
N-(4-fluorobenzyl)-2-[(1R,3R)-3-fluoro-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R,3R)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide; and pharmaceutically acceptable salts thereof.

Still another embodiment pertains to compounds of Formula (XIIb), selected from the group consisting of:
N-benzyl-2-(5′-bromo-2,5-dioxo-2′,3′-dihydrospiro[imidazolidine-4,1′-indene]-1-yl)-N-(1-cyclopropylethyl)acetamide;
2-(5′-bromo-2,5-dioxo-2′,3′-dihydrospiro[imidazolidine-4,1′-indene]-1-yl)-N-(1-cyclopropylethyl)-N-(furan-2-ylmethyl)acetamide;
N-benzyl-N-(1-cyclopropylethyl)-2-(5′-fluoro-2,5-dioxo-2′,3′-dihydrospiro[imidazolidine-4,1′-indene]-1-yl)acetamide;
N-benzyl-N-(1-cyclopropylethyl)-2-(5′-methoxy-2,5-dioxo-2′,3′-dihydrospiro[imidazolidine-4,1′-indene]-1-yl)acetamide;
N-benzyl-2-(5′-cyano-2,5-dioxo-2′,3′-dihydrospiro[imidazolidine-4,1′-indene]-1-yl)-N-(1-cyclopropylethyl)acetamide;
N-benzyl-2-(5′-bromo-2,5-dioxo-2′,3′-dihydrospiro[imidazolidine-4,1′-indene]-1-yl)-N-(1-cyclobutylethyl)acetamide;
2-(5′-bromo-2,5-dioxo-2′,3′-dihydrospiro[imidazolidine-4,1′-indene]-1-yl)-N-(1-cyclopropylethyl)-N-(4-fluorobenzyl)acetamide;
1-(2-(benzyl(1-cyclopropylethyl)amino)-2-oxoethyl)-2,5-dioxo-2′,3′-dihydrospiro[imidazolidine-4,1′-indene]-5′-carboxamide;
N-benzyl-N-(1-cyclopropylethyl)-2-(5′-(methylthio)-2,5-dioxo-2′,3′-dihydrospiro[imidazolidine-4,1′-indene]-1-yl)acetamide;
N-benzyl-N-(1-cyclopropylethyl)-2-(5′-(hydroxymethyl)-2,5-dioxo-2′,3′-dihydrospiro[imidazolidine-4,1′-indene]-1-yl)acetamide;
N-benzyl-N-(1-cyclopropylethyl)-2-(5′-((difluoromethoxy)methyl)-2,5-dioxo-2′,3′-dihydrospiro[imidazolidine-4,1′-indene]-1-yl)acetamide;
N-benzyl-2-(5′-cyclopropyl-2,5-dioxo-2′,3′-dihydrospiro[imidazolidine-4,1′-indene]-1-yl)-N-(1-cyclopropylethyl)acetamide;
N-benzyl-N-(1-cyclobutylethyl)-2-(5′-(difluoromethoxy)-2,5-dioxo-2′,3′-dihydrospiro[imidazolidine-4,1′-indene]-1-yl)acetamide;
2-(5′-acetamido-2,5-dioxo-2′,3′-dihydrospiro[imidazolidine-4,1′-indene]-1-yl)-N-benzyl-N-(1-cyclopropylethyl)acetamide;
N-benzyl-N-(1-cyclopropylethyl)-2-(5′-methyl-2,5-dioxo-2′,3′-dihydrospiro[imidazolidine-4,1′-indene]-1-yl)acetamide;
1-(2-(benzyl((S)-1-cyclopropylethyl)amino)-2-oxoethyl)-N-(cyanomethyl)-2,5-dioxo-2′,3′-dihydrospiro[imidazolidine-4,1′-indene]-5′-carboxamide;
N-benzyl-N-(1-cyclopropylethyl)-2-(5′-ethynyl-2,5-dioxo-2′,3′-dihydrospiro[imidazolidine-4,1′-indene]-1-yl)acetamide;
N-benzyl-N-(1-cyclopropylethyl)-2-(5′-(3-hydroxyprop-1-ynyl)-2,5-dioxo-2′,3′-dihydrospiro[imidazolidine-4,1′-indene]-1-yl)acetamide;
N-benzyl-N-(1-cyclopropylethyl)-2-(2,5-dioxo-5′-(1H-pyrazol-5-yl)-2′,3′-dihydrospiro[imidazolidine-4,1′-indene]-1-yl)acetamide;
N-benzyl-N-(1-cyclopropylethyl)-2-(2,5-dioxo-5′-(1H-1,2,3-triazol-4-yl)-2′,3′-dihydrospiro[imidazolidine-4,1′-indene]-1-yl)acetamide;
2-(5′-(1H-imidazol-2-yl)-2,5-dioxo-2′,3′-dihydrospiro[imidazolidine-4,1′-indene]-1-yl)-N-benzyl-N-(1-cyclopropylethyl)acetamide;
N-benzyl-N-(1-cyclopropylethyl)-2-(2,5-dioxo-5′-(1H-1,2,3-triazol-1-yl)-2′,3′-dihydrospiro[imidazolidine-4,1′-indene]-1-yl)acetamide;
N-benzyl-N-(1-cyclopropylethyl)-2-(2,5-dioxo-5′-(2H-1,2,3-triazol-2-yl)-2′,3′-dihydrospiro[imidazolidine-4, 1′-indene]-1-yl)acetamide;
N-benzyl-N-(1-cyclopropylethyl)-2-(5′-(methoxymethyl)-2,5-dioxo-2′,3′-dihydrospiro[imidazolidine-4,1′-indene]-1-yl)acetamide;
N-benzyl-2-(5′-(2-cyanoacetamido)-2,5-dioxo-2′,3′-dihydrospiro[imidazolidine-4,1′-indene]-1-yl)-N—((S)-1-cyclopropylethyl)acetamide;
methyl 1-(2-(benzyl((S)-1-cyclopropylethyl)amino)-2-oxoethyl)-2,5-dioxo-2′,3′-dihydrospiro[imidazolidine-4,1′-indene]-5′-ylcarbamate;
N-benzyl-N—((S)-1-cyclopropylethyl)-2-(5′-(3-methylureido)-2,5-dioxo-2′,3′-dihydrospiro[imidazolidine-4,1′-indene]-1-yl)acetamide;
N-benzyl-N—((S)-1-cyclopropylethyl)-2-(5′-(3-methyl-3-(methylcarbamoyl)ureido)-2,5-dioxo-2′,3′-dihydrospiro[imidazolidine-4,1′-indene]-1-yl)acetamide;
N-benzyl-N—((S)-1-cyclopropylethyl)-2-(5′-(3-hydroxy-3-methylbut-1-ynyl)-2,5-dioxo-2′,3′-dihydrospiro[imidazolidine-4,1′-indene]-1-yl)acetamide;
N-((1-(2-amino-2-oxoethyl)-1H-1,2,3-triazol-4-yl)methyl)-N-((S)-1-cyclobutylethyl)-2-((S)-2,5-dioxo-2′,3′-dihydrospiro[imidazolidine-4,1′-indene]-1-yl)acetamide;
(S)—N-benzyl-N-(1-cyclopropylethyl)-2-(2,5-dioxo-5′-(6-oxo-1,6-dihydropyridin-3-yl)-2′,3′-dihydrospiro[imidazolidine-4,1′-indene]-1-yl)acetamide;
N-benzyl-N—((S)-1-cyclopropylethyl)-2-((S)-2,5-dioxo-5′-(1H-pyrazol-3-yl)-2′,3′-dihydrospiro[imidazolidine-4,1′-indene]-1-yl)acetamide;
N-benzyl-N—((S)-1-cyclopropylethyl)-2-(5′-(methylsulfonamido)-2,5-dioxo-2′,3′-dihydrospiro[imidazolidine-4,1′-indene]-1-yl)acetamide;
N-benzyl-2-(5′-(2-cyanoacetamido)-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(2,5-dioxo-5′-(2H-tetrazol-5-yl)-2′,3′-dihydrospiro[imidazolidine-4,1′-indene]-1-yl)acetamide;
N-benzyl-N—((S)-1-cyclopropylethyl)-2-((S)-2,5-dioxo-5′-(6-oxo-1,6-dihydropyridin-3-yl)-2′,3′-dihydrospiro[imidazolidine-4,1′-indene]-1-yl)acetamide;
N-benzyl-N—((S)-1-cyclopropylethyl)-2-((S)-5′-(3-hydroxyprop-1-ynyl)-2,5-dioxo-2′,3′-dihydrospiro[imidazolidine-4,1′-indene]-1-yl)acetamide;
N-benzyl-N—((S)-1-cyclopropylethyl)-2-((S)-2,5-dioxo-5′-(2H-1,2,3-triazol-4-yl)-2′,3′-dihydrospiro[imidazolidine-4,1′-indene]-1-yl)acetamide;
N-benzyl-N—((S)-1-cyclopropylethyl)-2-(5′-(3-hydroxypyrrolidin-1-yl)-2,5-dioxo-2′,3′-dihydrospiro[imidazolidine-4,1′-indene]-1-yl)acetamide;
N-benzyl-N-((S)-1-cyclopropylethyl)-2-((S)-5′-(3-hydroxy-3-methylbut-1-ynyl)-2,5-dioxo-2′,3′-dihydrospiro[imidazolidine-4,1′-indene]-1-yl)acetamide;
N-benzyl-2-((S)-5′-cyano-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-((S)-5′-(3-methylureido)-2,5-dioxo-2′,3′-dihydrospiro[imidazolidine-4,1′-indene]-1-yl)acetamide;
N-benzyl-N—((S)-1-cyclopropylethyl)-2-(5′-(3-hydroxy azetidin-1-yl)-2,5-dioxo-2′,3′-dihydrospiro[imidazolidine-4,1′-indene]-1-yl)acetamide;
N-benzyl-2-((S)-5′-(2′-cyanoacetamido)-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(5′-(2,4-dioxo-3,4-dihydropyrimidin-1(2H)-yl)-2,5-dioxo-2′,3′-dihydrospiro[imidazolidine-4,1′-indene]-1-yl)acetamide;
N-(1-(2-(benzyl((S)-1-cyclopropylethyl)amino)-2-oxoethyl)-2,5-dioxo-2′,3′-dihydrospiro[imidazolidine-4,1′-indene]-5′-ylacrylamide;
N-benzyl-2-(5′-(2-cyano-1-hydroxyallyl)-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(5′-(2,4-dioxo-1,2,3,4-tetrahydropyrimidin-5-yl)-2,5-dioxo-2′,3′-dihydrospiro[imidazolidine-4,1′-indene]-1-yl)acetamide;
1-(2-(benzyl((S)-1-cyclopropylethyl)amino)-2-oxoethyl)-2,5-dioxo-2′,3′-dihydrospiro[imidazolidine-4,1′-indene]-5′-carboxylic acid;
(S)—N-(1-(2-(benzyl(1-cyclopropylethyl)amino)-2-oxoethyl)-2,5-dioxo-2′,3′-dihydrospiro[imidazolidine-4,1′-indene]-5′-yl)-3-oxobutanamide;
N-(1-(2-(benzyl((S)-1-cyclopropylethyl)amino)-2-oxoethyl)-2,5-dioxo-2′,3′-dihydrospiro[imidazolidine-4,1′-indene]-5′-yl)-2-cyano-3-oxobutanamide;
(E)-N-(1-(2-(benzyl((S)-1-cyclopropylethyl)amino)-2-oxoethyl)-2,5-dioxo-2′,3′-dihydrospiro[imidazolidine-4,1′-indene]-5′-yl)-4-(piperidin-1-yl)but-2-enamide;
2-amino-N-((1′S)-1-((4S)-3-benzyl-4-cyclopropyl-2-oxopentyl)-2,5-dioxo-2′,3′-dihydrospiro[imidazolidine-4,1′-indene]-5′-yl)-2-methylpropanamide;
(S)-2-amino-N—((S)-1-(2-(benzyl((S)-1-cyclopropylethyl)amino)-2-oxoethyl)-2,5-dioxo-2′,3′-dihydrospiro[imidazolidine-4,1′-indene]-5′-yl)propanamide;
N-benzyl-N—((S)-1-cyclopropylethyl)-2-(5′-(2-hydroxy-5-oxo-2,5-dihydrofuran-2-yl)-2,5-dioxo-2′,3′-dihydrospiro[imidazolidine-4,1′-indene]-1-yl)acetamide;
5′-(azetidin-3-ylmethylamino)-1-((4S)-3-benzyl-4-cyclopropyl-2-oxopentyl)-2′,3′-dihydrospiro[imidazolidine-4,1′-indene]-2,5-dion e;
1-amino-N—((S)-1-(2-(benzyl((S)-1-cyclopropylethyl)amino)-2-oxoethyl)-2,5-dioxo-2′,3′-dihydrospiro[imidazolidine-4,1′-indene]-5′-yl)cyclobutanecarboxamide;
N-benzyl-N—((S)-1-cyclopropylethyl)-2-(2,5-dioxo-5′-(thiazol-2-ylamino)-2′,3′-dihydrospiro[imidazolidine-4,1′-indene]-1-yl)acetamide;
(S)-2-(5′-(azetidin-3-ylamino)-2,5-dioxo-2′,3′-dihydrospiro[imidazolidine-4,1′-indene]-1-yl)-N-benzyl-N-(1-cyclopropylethyl)acetamide;
N-benzyl-2-[5′-(5-cyano-6-oxo-1,6-dihydropyridin-3-yl)-2,5-dioxo-2′,3′-dihydrospiro[imidazolidine-4,1′-indene]-1-yl]-N-(1-cyclopropylethyl)acetamide;
(2R)-2-amino-N-[(4S)-1-({benzyl[(1S)-1-cyclopropylethyl]carbamoyl}methyl)-2,5-dioxo-2′,3′-dihydrospiro[imidazolidine-4,1′-indene]-5′-yl]propanamide;
N-benzyl-N-[(1S)-1-cyclopropylethyl]-2-{5′-[(1H-imidazol-2-yl)amino]-2,5-dioxo-2′,3′-dihydrospiro[imidazolidine-4,1′-indene]-1-yl}acetamide;
1-amino-N-[(4S)-1-({benzyl[(1S)-1-cyclopropylethyl]carbamoyl}methyl)-2,5-dioxo-2′,3′-dihydrospiro[imidazoline-4,1′-indene]-5′-yl]cyclopropane-1-carboxamide;
N-benzyl-N-[(1S)-1-cyclopropylethyl]-2-{5′-[(oxetan-3-ylmethyl)amino]-2,5-dioxo-2′,3′-dihydrospiro[imidazolidine-4,1′-indene]-1-yl}acetamide;
2-[5′-(azetidin-3-yloxy)-2,5-dioxo-2′,3′-dihydrospiro[imidazolidine-4,1′-indene]-1-yl]-N-benzyl-N-[(1S)-1-cyclopropylethyl]acetamide;
N-benzyl-N-[(1R)-1-cyclopropyl-2,2,2-trifluoroethyl]-2-{1,1,2′,5′-tetraoxo-2H-spiro[1λ⁶-benzothiophene-3,4′-imidazolidine]-1′-yl}acetamide;
N-benzyl-2-{6-bromo-1,1,2′,5′-tetraoxo-2H-spiro[1λ⁰-benzothiophene-3,4′-imidazolidine]-1′-yl}-N-[(1S)-1-cyclopropylethyl]acetamide;
N-benzyl-N-[(1S)-1-cyclopropylethyl]-2-[1,1,2′,5′-tetraoxo-6-(1H-pyrazol-3-yl)-2H-spiro[1λ⁶-benzothiophene-3,4′-imidazolidine]-1′-yl]acetamide;
2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide;
2-(6-bromo-1,1-dioxido-2′,5′-dioxo-1′H-spiro[1-benzothiophene-3,4′-imidazolidin]-1-yl)-N-[(1S)-1-cyclopropylethyl]-N-(4-fluorobenzyl)acetamide;
N-[(1S)-1-cyclopropylethyl]-2-[2,5-dioxo-5′-(6-oxo-1,6-dihydropyridin-3-yl)-1,3′-dihydro-1H-spiro[imidazolidine-4,2′-inden]-1-yl]-N-(4-fluorobenzyl)acetamide;
2-(5′-amino-2,5-dioxo-1′,3′-dihydro-1H-spiro[imidazolidine-4,2′-inden]-1-yl)-N-[(1S)-1-cyclopropylethyl]-N-(4-fluorobenzyl)acetamide;
N-[(1S)-1-cyclopropylethyl]-2-[1,1-dioxido-2′,5′-dioxo-6-(6-oxo-1,6-dihydropyridin-3-yl)-1′H-spiro[1-benzothiophene-3,4′-imidazolidin]-1′-yl]-N-(4-fluorobenzyl)acetamide;
2-(6-amino-1,1-dioxido-2′,5′-dioxo-1′H-spiro[1-benzothiophene-3,4′-imidazolidin]-1′-yl)-N-[(1S)-1-cyclopropylethyl]-N-(4-fluorobenzyl)acetamide;
N-[(1S)-1-cyclopropylethyl]-N-(4-fluorobenzyl)-2-{5′-[(methylcarbamoyl)amino]-2,5-dioxo-1′,3′-dihydro-1H-spiro[imidazolidine-4,2′-inden]-1-yl}acetamide;
2-[5′-(acetylamino)-2,5-dioxo-1′,3′-dihydro-1H-spiro[imidazolidine-4,2′-inden]-1-yl]-N-[(1S)-1-cyclopropylethyl]-N-(4-fluorobenzyl)acetamide;
N-[(1S)-1-cyclopropylethyl]-N-(4-fluorobenzyl)-2-{6-[(methylcarbamoyl)amino]-1,1-dioxido-2′,5′-dioxo-1′H-spiro[1-benzothiophene-3,4′-imidazolidin]-1′-yl}acetamide;
2-[6-(acetylamino)-1,1-dioxido-2′,5′-dioxo-1′H-spiro[1-benzothiophene-3,4′-imidazolidin]-1′-yl]-N-[(1S)-1-cyclopropylethyl]-N-(4-fluorobenzyl)acetamide;
2-(6-bromo-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide;
2-(5′-bromo-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-{5′-[1-(2-amino-2-oxoethyl)-1H-pyrazol-4-yl]-2,3′,5-trioxo-2′,3′-dihydro-1H-spiro[imidazolidine-4,1′-inden]-1-yl}-N-(4-fluorobenzyl)-N-[(2S)-1,1,1-trifluoropropan-yl]acetamide;
N-(4-fluorobenzyl)-N-[(2S)-1,1,1-trifluoropropan-2-yl]-2-[2,3′,5-trioxo-5′-(6-oxo-1,6-dihydropyridin-3-yl)-2′,3′-dihydro-1H-spiro[imidazolidine-4,1′-inden]-1-yl]acetamide;
2-(7′-bromo-1′-methyl-2,5-dioxo-2′,3′-dihydro-1H,1′H-spiro[imidazolidine-4,4′-quinolin]-1-yl)-N-[(1S)-1-cyclopropylethyl]-N-(4-fluorobenzyl)acetamide;
2-[5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-(6-{1-[2-(dimethylamino)-2-oxoethyl]-1H-pyrazol-4-yl}-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide;
2-[5′-(acetylamino)-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[5′-(acetylamino)-2,5-dioxo-2′,3′-dihydro-1H-spiro[imidazolidine-4,1′-inden]-1-yl]-N-[(1S)-1-cyclopropylethyl]-N-(4-fluorobenzyl)acetamide;
N-(4-fluorobenzyl)-2-(5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-(3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
2-(6′-bromo-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
2-(6′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
2-(7′-amino-1′-methyl-2,5-dioxo-2′,3′-dihydro-1H,1′H-spiro[imidazolidine-4,4′-quinolin]-1-yl)-N-[(1S)-1-cyclopropylethyl]-N-(4-fluorobenzyl)acetamide;
N-[(1S)-1-cyclopropylethyl]-N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}acetamide;
N-[(1S)-1-cyclopropylethyl]-N-(4-fluorobenzyl)-2-{6′-[(methylcarbamoyl)amino]-2,5-dioxo-3′,4′-dihydro-1H,1′H-spiro[imidazolidine-4,2′-naphthalen]-1-yl}acetamide;
2-[6′-(acetylamino)-2,5-dioxo-3′,4′-dihydro-1H,1′H-spiro[imidazolidine-4,2′-naphthalen]-1-yl]-N-[(1S)-1-cyclopropylethyl]-N-(4-fluorobenzyl)acetamide;
2-(6-amino-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide;
N-(4-fluorobenzyl)-2-(6-{1-[2-(methylamino)-2-oxoethyl]-1H-pyrazol-4-yl}-1,1-dioxido-2′,5′-dioxo-1′H-spiro[1-benzothiophene-3,4′-imidazolidin]-1′-yl)-N-[(2S)-1,1,1-trifluoropropan-yl]acetamide;
tert-butyl {4-[1-(2-{(4-fluorobenzyl)[(2S)-1,1,1-trifluoropropan-2-yl]amino}-2-oxoethyl)-2,3′,5-trioxo-2′,3′-dihydrospiro[imidazolidine-4,1′-inden]-5′-yl]-1H-pyrazol-1-yl}acetate;
N-(4-fluorobenzyl)-2-{3′-fluoro-5′-[1-(2-methylpropyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
{4-[1-(2-{(4-fluorobenzyl)[(2S)-1,1,1-trifluoropropan-2-yl]amino}-2-oxoethyl)-2,3′,5-trioxo-2′,3′-dihydrospiro[imidazolidine-4,1′-inden]-5′-yl]-1H-pyrazol-1-yl}acetic acid;
2-(5′-{1-[2-(dimethylamino)-2-oxoethyl]-1H-pyrazol-4-yl}-2,3′,5-trioxo-2′,3′-dihydro-1H-spiro[imidazolidine-4,1′-inden]-1-yl)-N-(4-fluorobenzyl)-N-[(2)-1,1,1-trifluoropropan-2-yl]acetamide;
N-(4-fluorobenzyl)-2-(5′-{1-[2-(3-hydroxyazetidin-1-yl)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-{6-[(methylcarbamoyl)amino]-1,1-dioxido-2′,5′-dioxo-1′H-spiro[1-benzothiophene-3,4′-imidazolidin]-1′-yl}-N-[(2S)-1,1,1-trifluoropropan-2-yl]acetamide;
2-(5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-{5′-[1-(2-amino-2-oxoethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-{5′-[1-(2′-amino-2′-oxoethyl)-1H-pyrazol-4-yl]-3′-hydroxy-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-[(4S)-5′-{1-[2-(dimethylamino)-2-oxoethyl]-1H-pyrazol-4-yl}-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4S)-5′-{1-[2-(dimethylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[5′-(1-methyl-1H-pyrazol-4-yl)-2,3′,5-trioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(7′-bromo-1′-methyl-2,5-dioxo-2′,3′-dihydro-1H,1′H-spiro[imidazolidine-4,4′-quinolin]-1-yl)-N-[(1S)-1-cyclopropyl-2,2,2-trifluoroethyl]-N-(4-fluorobenzyl)acetamide;
N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}-N-[(2S)-1,1,1-trifluoropropan-2-yl]acetamide;
2-(7′-bromo-2,2′,5-trioxo-2′,3′-dihydro-1H,1′H-spiro[imidazolidine-4,4′-quinolin]-1-yl)-N-(4-fluorobenzyl)-N-[(2S)-1,1,1-trifluoropropan-2-yl]acetamide;
N-(4-fluorobenzyl)-2-(7′-{1-[2-(methylamino)-2-oxoethyl]-1H-pyrazol-4-yl}-2,2′,5-trioxo-2′,3′-dihydro-1H,1′H-spiro[imidazolidine-4,4′-quinolin]-1-yl)-N-[(2S)-1,1,1-trifluoropropan-2-yl]acetamide;
2-{7′-[1-(2-amino-2-oxoethyl)-1H-pyrazol-4-yl]-2,2′,5-trioxo-2′,3′-dihydro-1H,1′H-spiro[imidazolidine-4,4′-quinolin]-1-yl}-N-(4-fluorobenzyl)-N-[(2S)-1,1,1-trifluoropropan-2-yl]acetamide;
N-[(1S)-1-cyclopropyl-2,2,2-trifluoroethyl]-N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}acetamide;
2-(5′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2,2,2-trifluoro-N-[1-(2-{(4-fluorobenzyl)[(2S)-1,1,1-trifluoropropan-2-yl]amino}-2-oxoethyl)-1′-methyl-2,5-dioxo-2′,3′-dihydro-1′H-spiro[imidazolidine-4,4′-quinolin]-7′-yl]acetamide;
N-[1-(2-{[(1S)-1-cyclopropylethyl](4-fluorobenzyl)amino}-2-oxoethyl)-1′-methyl-2,5-dioxo-2′,3′-dihydro-1′H-spiro[imidazolidine-4,4′-quinolin]-7′-yl]-2,2,2-trifluoroacetamide;
N-(4-fluorobenzyl)-2-[3′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(5′-bromo-3′-hydroxy-2,5-dioxo-2′,3′-dihydro-1H-spiro[imidazolidine-4,1′-indene]-1-yl)-N-(4-fluorobenzyl)-N-[(2S)-1,1,1-trifluoropropan-2-yl]acetamide;
N-(4-fluorobenzyl)-2-{5′-[(methylcarbamoyl)amino]-2,3′,5-trioxo-2′,3′-dihydro-1H-spiro[imidazolidine-4,1′-inden]-1-yl}-N-[(2S)-1,1,1-trifluoropropan-2-yl]acetamide;
2-(5′-amino-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide;
2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{3′-hydroxy-5′-[1-(2-methylpropyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(4S)-5′-bromo-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-(5′-bromo-3′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-{(4S)-5′-[1-(2-amino-2-oxoethyl)-1H-pyrazol-4-yl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-[(1S)-1-cyclopropylethyl]-N-(4-fluorobenzyl)-2-[1′-methyl-7′-(1-methyl-1H-pyrazol-4-yl)-2,5-dioxo-2′,3′-dihydro-1H,1∝H-spiro[imidazolidine-4,4′-quinolin]-1-yl]acetamide;
N-(4-fluorobenzyl)-2-[7′-(1-methyl-1H-pyrazol-4-yl)-2,2′,5-trioxo-2′,3′-dihydro-1H,1′H-spiro[imidazolidine-4,4′-quinolin]-1-yl]-N-[(2S)-1,1,1-trifluoropropan-2-yl]acetamide;
N-(4-fluorobenzyl)-2-[(4S)-5′-(1-methyl-1H-pyrazol-4-yl)-2,3′,5-trioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(3′S,48)-3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(4S)-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(3′S,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(4S)-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(3′S,4S)-3′-hydroxy-5′-{1-[2-(methylamino)-2-oxoethyl]-1H-pyrazol-4-yl}-2,5-dioxo-2′,3′-dihydro-1′H-spiro[imidazolidine-4,1′-indene]-1-yl]-N-[(2S)-1,1,1-trifluoropropan-2-yl]acetamide;
N-(3,5-difluorobenzyl)-2-[(3′R,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(3′S,4S)-5′-bromo-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(3′R,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(3′R,4S)-5′-bromo-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{(4S)-5′-[1-(2-amino-2-oxoethyl)-1H-pyrazol-4-yl]-2,3′,5-trioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-{(3′S,4S)-5′-[1-(2-amino-2-oxoethyl)-1H-pyrazol-4-yl]-3′-hydroxy-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-[(3′S,4S)-5′-{1-[2-(dimethylamino)-2-oxoethyl]-1H-pyrazol-4-yl}-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3′R,4S)-5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(4S)-5′-{1-[2-(methylamino)-2-oxoethyl]-1H-pyrazol-4-yl}-2,5-dioxo-1H-spiro[imidazolidine-4,1′-inden]-1-yl]-N-[(2S)-1,1,1-trifluoropropan-2-yl]acetamide;
N-(3,4-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1′-trifluoropropan-2-yl]acetamide;
N-(3,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(2,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-[(1S)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-[(1S)-1-cyclopropyl-2,2,2-trifluoroethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-[(1R)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-benzyl-N-[(1S)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-benzyl-N-[(1R)-1-cyclopropylethyl]-2-[(4S)-5′-(1-methyl-3′H-pyrazol-4-yl)-2,5-dioxo-2′,3′-dihydro-1H-spiro[imidazolidine-4,1′-inden]-3-yl]acetamide;
2-[(3′S,4S)-5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3S)-6-amino-1,1-dioxido-2′,5′-dioxo-1H-spiro[1-benzothiophene-3,4′-imidazolidin]-1′-yl]-N-(4-fluorobenzyl)-N-[(2S)-1,1,1-trifluoropropan-2-yl]acetamide;
2-[(3′R,4S)-5′-bromo-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-(5′-bromo-6′-fluoro-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-(5′-bromo-6′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-(6′-fluoro-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-(6′-fluoro-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
2-(3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[(3′R,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(3R)-6-amino-1,1-dioxido-2′,5′-dioxo-1′H-spiro[1-benzothiophene-3,4′-imidazolidin]-1′-yl]-N-(4-fluorobenzyl)-N-[(2S)-1,1,1-trifluoropropan-2-yl]acetamide;
2-{2,5-dioxo-5′-[(2,2,2-trifluoroethyl)amino]-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-[(4S)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4R)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{(3′R,4S)-5′-[1-(2-amino-2-oxoethyl)-H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-(5′-bromo-6′-fluoro-3′-hydroxy-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-(6′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
2-{5′-[1-(2-amino-2-oxoethyl)-1H-pyrazol-4-yl]-6′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-{6′-fluoro-5′-[1-(2-hydroxyethyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-[6′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(5′-bromo-3′,6′-difluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-{5′-[1-(2-amino-2-oxoethyl)-1H-pyrazol-4-yl]-3′,6′-difluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-{4-[3′,6′-difluoro-1-(2-{(4-fluorobenzyl) [(2S)-1,1,1-trifluoropropan-2-yl]amino}-2-oxoethyl)-2,5-dioxo-2′,3′-dihydro-1H-spiro[imidazolidine-4,1′-inden]-5′-yl]-1H-pyrazol-1-yl}-N,N-dimethylacetamide;
2-[3′,6′-difluoro-5′-(1-methyl-1H-pyrazol-4-yl)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{3′,6′-difluoro-5′-[1-(2-hydroxyethyl)-1H-pyrazol-4-yl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-[(4R)-5′-(1-methyl-1H-pyrazol-4-yl)-2,5-dioxo-2′,3′-dihydro-1H-spiro[imidazolidine-4,1′-inden]-1-yl]-N-[(2 S)-1,1,1-trifluoropropan-2-yl]acetamide;
2-[(4S)-2,5-dioxo-5′-(1H-pyrazol-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{5′-[(1-methyl-1H-pyrazol-3-yl)amino]-2,5-dioxo-2′,3′-dihydro-1H-spiro[imidazolidine-4,1′-inden]-1-yl}-N-[(2S)-1,1,1-trifluoropropan-2-yl]acetamide;
2-[(4S)-5′-amino-2,5-dioxo-2′,3′-dihydro-1H-spiro[imidazolidine-4, 1′-inden]-1-yl]-N-(4-fluorobenzyl)-N-[(2S)-1,1,1-trifluoropropan-2-yl]acetamide;
methyl trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylate;
trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylic acid;
trans-4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]cyclohexanecarboxylic acid;
trans-4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}cyclohexanecarboxylic acid;
ethyl ({4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]piperidin-1-yl}sulfonyl)carbamate;
2-[(4S)-5′-{[(2-cyanoethyl)carbamoyl]amino}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{(4S)-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(4S)-5′-[(methylcarbamoyl)amino]-2,3′,5-trioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(3′S,4S)-3′-hydroxy-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(3′R,4S)-3′-fluoro-5′-[1-(2-hydroxyethyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
2-[(4R)-5′-bromo-6′-fluoro-2,3′,5-trioxo-2′,3′-dihydro-1H-spiro[imidazolidine-4,1′-inden]-1-yl]-N-(4-fluorobenzyl)-N-[(2S)-1, 1,1-trifluoropropan-2-yl]acetamide;
N-(4-fluorobenzyl)-2-{(3′R,4S)-3′-fluoro-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{7′-[(methylcarbamoyl)amino]-2,2′,5-trioxo-2′,3′-dihydro-1H, 1′H-spiro[imidazolidine-4,4′-quinolin]-1-yl}-N-[(2S)-1,1,1-trifluoropropan-2-yl]acetamide;
tert-butyl (4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetate;
(4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetic acid;
{4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
(4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}cyclohexylidene)acetic acid;
N-(4-fluorobenzyl)-2-[(3′R,4S)-3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-[(4R)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(3′E,4S)-3′-(hydroxyimino)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-[(3′R,4S)-3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{4-[(3′R,4S)-3′,6′-difluoro-1-(2-{(4-fluorobenzyl) [(2S)-1,1,1-trifluoropropan-2-yl]amino}-2-oxoethyl)-2,5-dioxo-2′,3′-dihydro-H-spiro[imidazolidine-4,1′-inden]-5′-yl]-1H-pyrazol-1-yl}-N,N-dimethylacetamide;
2-[(3′S,4R)-3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{4-[(3′S,4R)-3′,6′-difluoro-1-(2-{(4-fluorobenzyl)[(2S)-1,1,1-trifluoropropan-2-yl]amino}-2-oxoethyl)-2,5-dioxo-2′,3′-dihydro-H-spiro[imidazolidine-4,1′-inden]-5′-yl]-1H-pyrazol-1-yl}-N,N-dimethylacetamide;
ethyl [(4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}piperidin-1-yl)sulfonyl]carbamate;
tert-butyl 4-[{[(4S)-5′-bromo-2,5-dioxo-2′,3′-dihydro-1H-spiro[imidazolidine-4,1′-inden]-1-yl]acetyl}(4-fluorobenzyl)amino]piperidine-1-carboxylate;
ethyl ({4-[{[(4S)-5′-amino-2,5-dioxo-2′,3′-dihydro-H-spiro[imidazolidine-4,1′-inden]-1-yl]acetyl}(4-fluorobenzyl)amino]piperidin-1-yl}sulfonyl)carbamate;
ethyl ({3,3-difluoro-4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]pyrrolidin-1-yl}sulfonyl)carbamate;
ethyl ({3,3-difluoro-4-[(4-fluorobenzyl) {[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]acetyl}amino]pyrrolidin-1-yl}sulfonyl)carbamate;
2-{(3′R,4S)-5′-[1-(difluoromethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
tert-butyl {4-[{[(4S)-5′-bromo-2,5-dioxo-2′,3′-dihydro-1H-spiro[imidazolidine-4,1′-inden]-1-yl]acetyl}(4-fluorobenzyl)amino]cyclohexylidene}acetate;
tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate;
{4-[{[(4S)-5′-amino-2,5-dioxo-2′,3′-dihydro-1H-spiro[imidazolidine-4,1′-inden]-1-yl]acetyl}(4-fluorobenzyl)amino]cyclohexylidene}acetic acid;
2-[(4S)-5′-bromo-6′-fluoro-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3′R,4R)-5′-bromo-6′-fluoro-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3′S,4S)-5′-bromo-6′-fluoro-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]-N-(tetrahydro-2H-pyran-4-yl)acetamide;
N-(4-fluorobenzyl)-2-{6′-fluoro-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
2-{(3′R,4S)-3′,6′-difluoro-5′-[(methylcarbamoyl)amino]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
methyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate;
{4-[(4-fluorobenzyl) {[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetate;
{4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetic acid;
2-[(4S)-2,5-dioxo-5′-(1H-pyrazol-5-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
4-[(4-fluorobenzyl) {[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexanecarboxylic acid;
2-[(4S)-5′-{1-[2-(dimethylamino)ethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
tert-butyl 4-({4-[(4S)-1-(2-{(4-fluorobenzyl)[(2S)-1,1,1-trifluoropropan-2-yl]amino}-2-oxoethyl)-2,5-dioxo-2′,3′-dihydrospiro[imidazolidine-4,1′-inden]-5′-yl]-1H-pyrazol-1-yl}methyl)piperidine-1-carboxylate;
2-{(4S)-2,5-dioxo-5′-[1-(piperidin-4-ylmethyl)-1H-pyrazol-4-yl]-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-{6-[(1-methyl-1H-pyrazol-4-yl)amino]-1,1-dioxido-2′,5′-dioxo-1′H-spiro[1-benzothiophene-3,4′-imidazolidin]-1′-yl}-N-[(2S)-1,1,1-trifluoropropan-2-yl]acetamide;
N-(4-fluorobenzyl)-2-(6-methoxy-1,1-dioxido-2′,5′-dioxo-1′H-spiro[1-benzothiophene-3,4′-imidazolidin]-1′-yl)-N-[(2S)-1,1,1-trifluoropropan-2-yl]acetamide;
N-(4-fluorobenzyl)-2-[(3 S)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide;
N-(4-fluorobenzyl)-2-[(3R)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide;
N-benzyl-N-(1-cyclopropylethyl)-2-[5′-(methylsulfonyl)-2,5-dioxo-2′,3′-dihydro-1H-spiro[imidazolidine-4,1′-inden]-1-yl]acetamide;
N-benzyl-N-(1-cyclopropylethyl)-2-[5′-(hydroxymethyl)-3-methyl-2,5-dioxo-2′,3′-dihydro-1H-spiro[imidazolidine-4,1′-inden]-1-yl]acetamide;
N-benzyl-N-(1-cyclopropylethyl)-2-[5′-(6-hydroxypyridin-3-yl)-2,5-dioxo-2′,3′-dihydro-1H-spiro[imidazolidine-4,1′-inden]-1-yl]acetamide;
N-benzyl-N-[(1S)-1-cyclopropylethyl]-2-[5′-(formylamino)-2,5-dioxo-2′,3′-dihydro-1H-spiro[imidazolidine-4,1′-inden]-1-yl]acetamide;
N-benzyl-2-[(4R)-5′-bromo-2,5-dioxo-2′,3′-dihydro-1H-spiro[imidazolidine-4,1′-inden]-1-yl]-N-[(1S)-1-cyclopropylethyl]acetamide;
2-[1,1-dioxido-2′,5′-dioxo-6-(pyridin-3-yl)-1′H-spiro[1-benzothiophene-3,4′-imidazolidin]-1′-yl]-N-(4-fluorobenzyl)-N-[(2S)-1,1,1-trifluoropropan-2-yl]acetamide;
N-(4-fluorobenzyl)-2-[(3′S,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
(4-{[(5′-bromo-6′-fluoro-2,5-dioxo-2′,3′-dihydro-1H-spiro[imidazolidine-4,1′-inden]-1-yl)acetyl](4-fluorobenzyl)amino}cyclohexylidene)acetic acid;
{4-[(4-fluorobenzyl) {[6′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
tert-butyl (4-{[(5′-bromo-6′-fluoro-2,5-dioxo-2′,3′-dihydro-1H-spiro[imidazolidine-4,1′-inden]-1-yl)acetyl](4-fluorobenzyl)amino}cyclohexylidene)acetate; and pharmaceutically acceptable salts thereof.

In another aspect, the present invention relates to compounds of Formula (XIIIa) or Formula (XIIIb),

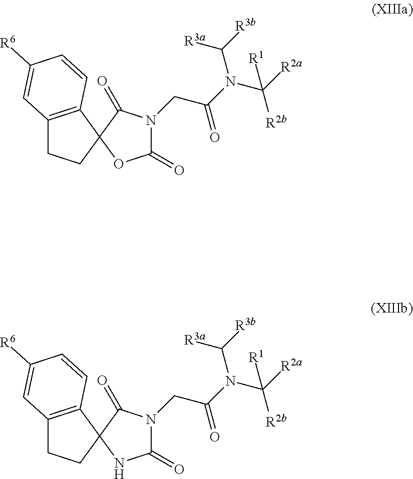

or a stereoisomer, tautomer or pharmaceutically acceptable salt thereof,
wherein
R¹ is carbocyclyl or heterocyclyl;
R^{2a} and R^{2b} are each independently H, D, or C₁-C₆ alkyl;
R^{3a} is hydrogen, C(O)NH₂, C₁-C₆ alkyl, aryl, cycloalkyl or heterocyclyl; and
R^{3b} is C₁-C₆ alkyl, aryl, cycloalkyl or heterocyclyl; or
R^{3a} and R^{3b} taken together with the carbon to which they are attached form an arene, cycloalkane, or heterocycle;
R⁶ is independently halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═S)N(R¹²)₂, —NHC(═N—C≡N)NR¹², —NHC(═N—C≡N)SR¹², or —NHS(O)ₘR¹²;
R¹¹ and R¹³ are each independently H, —OH, or C₁-C₆ alkyl; and
R¹², at each occurrence, is independently H, C₁-C₆ alkyl, aryl, cycloalkyl, or heterocyclyl;

with the proviso that R^{3a} and R^{3b} taken together with the carbon to which they are attached do not form tetrahydrothiophene 1,1-dioxide or tetrahydrothiophene when R¹ is unsubstituted phenyl.

In one embodiment of Formula (XIIIa) or (XIIIb), R¹ is carbocyclyl or heterocyclyl. In another embodiment of Formula (XIIIa) or (XIIIb), R¹ is carbocyclyl. In another embodiment of Formula (XIIIa) or (XIIIb), R¹ is heterocyclyl. In another embodiment of Formula (XIIIa) or (XIIIb), R¹ is phenyl, naphthyl, cyclopropyl, or cyclobutyl. In another embodiment of Formula (XIIIa) or (XIIIb), R¹ is phenyl. In another embodiment of Formula (XIIIa) or (XIIIb), R¹ is phenyl, which is unsubstituted. In another embodiment of Formula (XIIIa) or (XIIIb), R¹ is phenyl, which is substituted. In another embodiment of Formula (XIIIa) or (XIIIb), R¹ is phenyl, which is substituted with F, Cl, Br, CN, —NR_{g}Rₕ, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, aryl, and/or haloalkyl; wherein alkyl, alone or part of a group, is optionally substituted with F, C(═O)NR_{g}Rₕ, or CN. In another embodiment of Formula (XIIIa) or (XIIIb), R¹ is phenyl, which is substituted with F, Cl, Br, CN, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, and/or haloalkyl; wherein alkyl, alone or part of a group, is optionally substituted with F. In another embodiment of Formula (XIIIa) or (XIIIb), R¹ is phenyl, which is substituted with F. In another embodiment of Formula (XIIIa) or (XIIIb), R¹ is tetrahydropyranyl, thiazolyl, oxazolyl, thiadiazolyl, pyrimidinyl, thiophenyl, furanyl, triazolyl, indolyl, imidazolyl, or pyridinyl. In another embodiment of Formula (XIIIa) or (XIIIb), R¹ is tetrahydropyranyl, thiazolyl, oxazolyl, thiadiazolyl, pyrimidinyl, thiophenyl, furanyl, triazolyl, indolyl, imidazolyl, or pyridinyl; which is unsubstituted. In another embodiment of Formula (XIIIa) or (XIIIb), R¹ is tetrahydropyranyl, thiazolyl, oxazolyl, thiadiazolyl, pyrimidinyl, thiophenyl, furanyl, triazolyl, indolyl, imidazolyl, or pyridinyl; which is substituted. In another embodiment of Formula (XIIIa) or (XIIIb), R¹ is tetrahydropyranyl, thiazolyl, oxazolyl, thiadiazolyl, pyrimidinyl, thiophenyl, furanyl, triazolyl, indolyl, imidazolyl, or pyridinyl; which is substituted with F, Cl, Br, CN, —NR_{g}Rₕ, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, aryl, and/or haloalkyl; wherein alkyl, alone or part of a group, is optionally substituted with F, C(═O)NR_{g}Rₕ, or CN.

In one embodiment of Formula (XIIIa) or (XIIIb), R^{2a} and R^{2b} are each independently H, D, or C₁-C₆ alkyl. In another embodiment of Formula (XIIIa) or (XIIIb), R^{2a} and R^{2b} are each independently H. In another embodiment of Formula (XIIIa) or (XIIIb), R^{2a} is H and R^{2b} is C₁-C₆ alkyl. In another embodiment of Formula (XIIIa) or (XIIIb), R^{2a} is H and R^{2b} is C₁-alkyl.

In one embodiment of Formula (XIIIa) or (XIIIb), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; or R^{3a} and R^{3b} taken together with the carbon to which they are attached form arene, cycloalkane, or heterocycle. In another embodiment of Formula (XIIIa) or (XIIIb), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; wherein C₁-C₆ alkyl, is optionally substituted with F, —CN, —NR_{g}Rₕ, —OR_{g}, or —SO₂R_{g}; wherein R_{g} and Rₕ are the same or different and independently hydrogen or alkyl; wherein aryl, cycloalkyl and heterocyclyl are optionally substituted with alkyl, —SO₂NR_{g}C(═O)ORₕ, —SO₂NR_{g}Rₕ, —C(═O)R_{g}, —C(═O)OR_{g}, or —C(═O)NR_{g}Rₕ; wherein R_{g} and Rₕ are the same or different and independently hydrogen or alkyl; wherein alkyl, alone or part of a group, is optionally substituted with aryl or —C(═O)OH.

In another embodiment of Formula (XIIIa) or (XIIIb), R^{3a} is C₁-C₆ alkyl; and R^{3b} is C₁-C₆ alkyl; wherein the C₁-C₆ alkyl is optionally substituted with one or more F, —CN, alkoxy, or SO₂R_{g}. In another embodiment of Formula (XIIIa) or (XIIIb), R^{3a} is C₁-C₆ alkyl; and R^{3b} is CF₃. In another embodiment of Formula (XIIIa) or (XIIIb), R^{3a} is CH₃; and R^{3b} is CF₃. In another embodiment of Formula (XIIIa) or (XIIIb), R^{3a} is hydrogen; and R^{3b} is C₁-C₆ alkyl; wherein the C₁-C₆ alkyl is optionally substituted with one or more F, —CN, alkoxy, or SO₂R_{g}. In another embodiment of Formula (XIIIa) or (XIIIb), R^{3a} is hydrogen; and R^{3b} is cycloalkyl. In another embodiment of Formula (XIIIa) or (XIIIb), R^{3a} is hydrogen; and R^{3b} is cyclopropyl. In another embodiment of Formula (XIIIa) or (XIIIb), R^{3a} is C₁-C₆ alkyl; wherein the C₁-C₆ alkyl is CH₃; and R^{3b} is cycloalkyl. In another embodiment of Formula (XIIIa) or (XIIIb), R^{3a} is CH₃; and R^{3b} is cyclopropyl. In another embodiment of Formula (XIIIa) or (XIIIb), R^{3a} is CF₃; and R^{3b} is cyclopropyl. In another embodiment of Formula (XIIIa) or (XIIIb), R^{3a} is cycloalkyl; and R^{3b} is cycloalkyl. In another embodiment of Formula (XIIIa) or (XIIIb), R^{3a} is cyclopropyl; and R^{3b} is cyclopropyl. In another embodiment of Formula (XIIIa) or (XIIIb), R^{3a} is C₁-C₆ alkyl; and R^{3b} is heterocyclyl. In another embodiment of Formula (XIIIa) or (XIIIb), R^{3a} is hydrogen; and R^{3b} is aryl. In another embodiment of Formula (XIIIa) or (XIIIb), R^{3a} and R^{3b} taken together with the carbon to which they are attached form arene, cycloalkane, or heterocycle. In another embodiment of Formula (XIIIa) or (XIIIb), R^{3a} and R^{3b} taken together with the carbon to which they are attached form arene. In another embodiment of Formula (XIIIa) or (XIIIb), R^{3a} and R^{3b} taken together with the carbon to which they are attached form cycloalkane. In another embodiment of Formula (XIIIa) or (XIIIb), R^{3a} and R^{3b} taken together with the carbon to which they are attached form heterocycle. In another embodiment of Formula (XIIIa) or (XIIIb), R^{3a} and R^{3b} taken together with the carbon to which they are attached form cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, or benzene. In another embodiment of Formula (XIIIa) or (XIIIb), R^{3a} and R^{3b} taken together with the carbon to which they are attached form cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, or benzene wherein cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, and benzene are optionally substituted with alkyl, haloalkyl, F, CN, —NR_{g}Rₕ, —NR_{g}C(═O)Rₕ, —NR_{g}SO₂Rₕ, —OR_{g}, —SO₂R_{g}, —NR_{g}SO₂NR_{g}C(═O)Rₕ, —SO₂NR_{g}C(═O)ORₕ, —SO₂NR_{g}Rₕ, —C(═O)R_{g}, —C(═O)OR_{g}, or —C(═O)NR_{g}Rₕ; wherein R_{g} and Rₕ are the same or different and independently hydrogen, alkyl, or haloalkyl; wherein each alkyl, alone or part of a group, is optionally substituted with CN, alkyl, —SO₂NH₂, C(═O)NHCH₃, —C(═O)OC(CH₃)₃, —C(═O)OCH₃, or —C(═O)OH.

In one embodiment of Formula (XIIIa) or (XIIIb), R⁶ is independently halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R²)₂, —NHC(═S)N(R¹²)₂, —NHC(═N—C≡N)NR¹², —NHC(═N—C≡N)SR¹², or —NHS(O)ₘR¹². In another embodiment of Formula (XIIIa) or (XIIIb), R⁶ is halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R²)₂, —NHC(═S)N(R¹²)₂, —NHC(═N—C≡N)NR¹², —NHC(═N—C≡N)SR¹², or —NHS(O)ₘR¹². In another embodiment of Formula (XIIIa) or (XIIIb), R⁶ is —N(R¹²)₂, —C(═O)NHR¹², —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═O)N(R¹²)₂, —NHC(═S)N(R¹²)₂, —NHC(═N—C≡N)NR¹², —NHC(═N—C≡N)SR¹², or —NHS(O)ₘR¹². In another embodiment of Formula (XIIIa) or (XIIIb), R⁶ is heterocyclyl. In another embodiment of Formula (XIIIa) or (XIIIb), R⁶ is pyrazolyl. In another embodiment of Formula (XIIIa) or (XIIIb), R⁶ is —NHC(═O)NHR¹².

In one embodiment of Formula (XIIIa) or (XIIIb), R¹¹ and R¹³ are each independently H, —OH, or C₁-C₆ alkyl. In another embodiment of Formula (XIIIa) or (XIIIb), R¹¹ and R¹³ are each independently H. In another embodiment of Formula (XIIIa) or (XIIIb), R¹¹ and R¹³ are each independently —OH. In another embodiment of Formula (XIIIa) or (XIIIb), R¹¹ and R¹³ are each independently C₁-C₆ alkyl.

In one embodiment of Formula (XIIIa) or (XIIIb), R¹², at each occurrence, is independently H, C₁-C₆ alkyl, aryl, cycloalkyl, or heterocyclyl. In another embodiment of Formula (XIIIa) or (XIIIb), R¹², at each occurrence, is independently H, C₁-C₆ alkyl, aryl, cycloalkyl, or heterocyclyl; wherein C₁-C₆ alkyl, aryl, cycloalkyl, and heterocyclyl are optionally substituted with F, alkyl, alkoxy, cycloalkyl, haloalkyl, heterocyclyl, heterocyclylalkyl, oxo, CN, —NR_{g}Rₕ, —NR_{g}C(═O)Rₕ, —NR_{g}C(═O)ORₕ, —OR_{g}, —SO₂R_{g}, —SO₂NR_{g}Rₕ, —C(═O)R_{g}, —C(═O)OR_{g}, or —C(═O)NR_{g}Rₕ; wherein R_{g} and Rₕ are the same or different and independently hydrogen, alkyl, alkoxy, aryl, cycloalkyl, and/or haloalkyl.

Still another embodiment pertains to compounds of Formula (XIIIa), selected from the group consisting of:
N-benzyl-2-(5-bromo-2′,4′-dioxo-2,3-dihydrospiro[indene-1,5′-ozazolidine]-3′-yl)-N-(1-cyclopropylethyl)acetamide;
2-(5-bromo-2′,4′-dioxo-2,3-dihydrospiro[indene-1,5′-ozazolidine]-3′-yl)-N-(1-cyclopropylethyl)-N-(4-fluorobenzyl)acetamide;
N-benzyl-2-(5-cyano-2′,4′-dioxo-2,3-dihydrospiro[indene-1,5′-ozazolidine]-3′-yl)-N-(1-cyclopropylethyl)acetamide;
(S)-2-(5-acetamido-2′,4′-dioxo-2,3-dihydrospiro[indene-1,5′-ozazolidine]-3′-yl)-N-benzyl-N-(1-cyclopropylethyl)acetamide;
3′-(2-(benzyl(1-cyclopropylethyl)amino)-2-oxoethyl)-2′,4′-dioxo-2,3-dihydrospiro[indene-1,5′-ozazolidine]-5-carboxamide;
N-benzyl-N-(1-cyclopropylethyl)-2-(5-methoxy-2′,4′-dioxo-2,3-dihydrospiro[indene-1,5′-ozazolidine]-3′-yl)acetamide;
N-benzyl-N—((S)-1-cyclopropylethyl)-2-(5-(3-hydroxyprop-1-ynyl)-2′,4′-dioxo-2,3-dihydrospiro[indene-1,5′-ozazolidine]-3′-yl)acetamide;
N-benzyl-N—((S)-1-cyclopropylethyl)-2-(5-(methylsulfonamido)-2′,4′-dioxo-2,3-dihydrospiro[indene-1,5′-ozazolidine]-3′-yl)acetamide;
(S)-3′-(2-(benzyl(1-cyclopropylethyl)amino)-2-oxoethyl)-2′,4′-dioxo-2,3-dihydrospiro[indene-1,5′-ozazolidine]-5-carboxamide;
N-benzyl-N—((S)-1-cyclopropylethyl)-2-(5-(morpholinomethyl)-2′,4′-dioxo-2,3-dihydrospiro[indene-1,5′-ozazolidine]-3′-yl)acetamide;
N-benzyl-N—((S)-1-cyclopropylethyl)-2-(5-(3-methylureido)-2′,4′-dioxo-2,3-dihydrospiro[indene-1,5′-ozazolidine]-3′-yl)acetamide;
(S)—N-(3′-(2-(benzyl(1-cyclopropylethyl)amino)-2-oxoethyl)-2′,4′-dioxo-2,3-dihydrospiro[indene-1,5′-ozazolidine]-5-yl)-3-oxobutanamide;
N-benzyl-N—((S)-1-cyclopropylethyl)-2-(5-morpholino-2′,4′-dioxo-2,3-dihydrospiro[indene-1,5′-ozazolidine]-3′-yl)acetamide;
N-benzyl-2-(5-(2-cyanoacetamido)-2′,4′-dioxo-2,3-dihydrospiro[indene-1,5′-ozazolidine]-3′-yl)-N—((S)-1-cyclopropylethyl)acetamide;
N-benzyl-N—((S)-1-cyclopropylethyl)-2-(2′,4′-dioxo-5-(2-oxa-6-azaspiro[3.3]heptan-6-yl)-2,3-dihydrospiro[indene-1,5′-ozazolidine]-3′-yl)acetamide;
N-benzyl-N—((S)-1-cyclopropylethyl)-2-(2′,4′-dioxo-5-(piperidin-1-ylmethyl)-2,3-dihydrospiro[indene-1,5′-ozazolidine]-3′-yl)acetamide;
N-benzyl-N—((S)-1-cyclopropylethyl)-2-(2′,4′-dioxo-5-(piperazin-1-ylmethyl)-2,3-dihydrospiro[indene-1,5′-ozazolidine]-3′-yl)acetamide;
N-benzyl-N—((S)-1-cyclopropylethyl)-2-(5-((4-hydroxypiperidin-1-yl)methyl)-2′,4′-dioxo-2,3-dihydrospiro[indene-1,5′-ozazolidine]-3′-yl)acetamide;
(S)-2-amino-N-(3′-(2-(benzyl(1-cyclopropylethyl)amino)-2-oxoethyl)-2′,4′-dioxo-2,3-dihydrospiro[indene-1,5′-ozazolidine]-5-yl)-2-methylpropanamide;
(S)—N-benzyl-N-(1-cyclopropylethyl)-2-(2′,4′-dioxo-5-(piperazin-1-yl)-2,3-dihydrospiro[indene-1,5′-ozazolidine]-3′-yl)acetamide;
N-benzyl-N—((S)-1-cyclopropylethyl)-2-(5-hydroxy-2′,4′-dioxo-2,3-dihydrospiro[indene-1,5′-ozazolidine]-3′-yl)acetamide;
methyl 2-(3′-(2-(benzyl((S)-1-cyclopropylethyl)amino)-2-oxoethyl)-2′,4′-dioxo-2,3-dihydrospiro[indene-1,5′-ozazolidine]-5-yloxyacetate;
N-benzyl-N—((S)-1-cyclopropylethyl)-2-(2′,4′-dioxo-5-(prop-2-ynyloxy)-2,3-dihydrospiro[indene-1,5′-ozazolidine]-3′-yl)acetamide;
(S)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)-3-methylbutanamide;
(2S)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)propanamide;
2-(3′-(2-(benzyl((S)-1-cyclopropylethyl)amino)-2-oxoethyl)-2′,4′-dioxo-2,3-dihydrospiro[indene-1,5′-ozazolidine]-5-ylamino)acetic acid;
N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-cyano-3-oxobutanamide;
2-(3′-(2-(benzyl((S)-1-cyclopropylethyl)amino)-2-oxoethyl)-2′,4′-dioxo-2,3-dihydrospiro[indene-1,5′-ozazolidine]-5-yloxy)acetic acid;
N-benzyl-N—((S)-1-cyclopropylethyl)-2-(5-(oxetan-3-ylamino)-2′,4′-dioxo-2,3-dihydrospiro[indene-1,5′-ozazolidine]-3′-yl)acetamide;
N-benzyl-N—((S)-1-cyclopropylethyl)-2-(2′,4′-dioxo-5-(tetrahydro-2H-pyran-4-ylamino)-2,3-dihydrospiro[indene-1,5′-ozazolidine]-3′-yl)acetamide;
N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-hydroxypropanamide;
1-(3′-(2-(benzyl((S)-1-cyclopropylethyl)amino)-2-oxoethyl)-2′,4′-dioxo-2,3-dihydrospiro[indene-1,5′-ozazolidine]-5-yl)azetidine-3-carboxamide;
2-amino-N-(3′-(2-(benzyl(dicyclopropylmethyl)amino)-2-oxoethyl)-2′,4′-dioxo-2,3-dihydrospiro[indene-1,5′-ozazolidine]-5-yl)-2-methylpropanamide;
N-benzyl-N-(dicyclopropylmethyl)-2-(5-(3-methylureido)-2′,4′-dioxo-2,3-dihydrospiro[indene-1,5′-ozazolidine]-3′-yl)acetamide;
N-benzyl-N—((S)-1-cyclopropylethyl)-2-(5-(2-(dimethylamino)ethoxy)-2′,4′-dioxo-2,3-dihydrospiro[indene-1,5′-ozazolidine]-3′-yl)acetamide;
2-(5-((1H-1,2,3-triazol-4-yl)methoxy)-2′,4′-dioxo-2,3-dihydrospiro[indene-1,5′-ozazolidine]-3′-yl)-N-benzyl-N—((S)-1-cyclopropylethyl)acetamide;
N-benzyl-N—((S)-1-cyclopropylethyl)-2-(2′,4′-dioxo-5-(6-oxo-1,6-dihydropyridin-3-yl)-2,3-dihydrospiro[indene-1,5′-ozazolidine]-3′-yl)acetamide;
N-benzyl-N—((S)-1-cyclopropylethyl)-2-(5-(2-hydroxyethoxy)-2′,4′-dioxo-2,3-dihydrospiro[indene-1,5′-ozazolidine]-3′-yl)acetamide;
3′-(2-(benzyl((S)-1-cyclopropylethyl)amino)-2-oxoethyl)-2′,4′-dioxo-2,3-dihydrospiro[indene-1,5′-ozazolidine]-5-ylboronic acid;
N-benzyl-N—((S)-1-cyclopropylethyl)-2-(5-(3-(hydroxymethyl)azetidin-1-yl)-2′,4′-dioxo-2,3-dihydrospiro[indene-1,5′-ozazolidine]-3′-yl)acetamide;
N—((S)-1-cyclopropylethyl)-2-(2′,4′-dioxo-5-(6-oxo-1,6-dihydropyridin-3-yl)-2,3-dihydrospiro[indene-1,5′-ozazolidine]-3′-yl)-N-(4-fluorobenzyl)acetamide;
1-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)cyclobutanecarboxamide;
2-(5-(2-cyanoacetamido)-2′,4′-dioxo-2,3-dihydrospiro[indene-1,5′-ozazolidine]-3′-yl)-N—((S)-1-cyclopropylethyl)-N-(4-fluorobenzyl)acetamide;
N—((S)-1-cyclopropylethyl)-N-(4-fluorobenzyl)-2-(5-(3-methylureido)-2′,4′-dioxo-2,3-dihydrospiro[indene-1,5′-ozazolidine]-3′-yl)acetamide;
3′-((4S)-3-benzyl-4-cyclopropyl-2-oxopentyl)-5-(1H-pyrazol-5-yl)-2,3-dihydrospiro[indene-1,5′-ozazolidine]-2′,4′-dione;
2-amino-N-(3′-(2-(benzyl((S)-1-cyclopropylethyl)amino)-2-oxoethyl)-4′-oxo-2,3-dihydrospiro[indene-1,5′-ozazolidine]-5-yl)-3-hydroxypropanamide;
(S)—N-(3′-(2-(benzyl(1-cyclopropylethyl)amino)-2-oxoethyl)-2′,4′-dioxo-2,3-dihydrospiro[indene-1,5′-ozazolidine]-5-yl)-2-hydroxy-2-methylpropanamide;
1-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)cyclopropanecarboxamide;
2-amino-N-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)-2-methylpropanamide;
(S)-2-(5-(azetidin-3-ylamino)-2′,4′-dioxo-2,3-dihydrospiro[indene-1,5′-ozazolidine]-3′-yl)-N-benzyl-N-(1-cyclopropylethyl)acetamide;
(2R)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-ylpropanamide;
(S)-2-(3′-(2-(benzyl(1-cyclopropylethyl)amino)-2-oxoethyl)-2′,4′-dioxo-2,3-dihydrospiro[indene-1,5′-ozazolidine]-5-ylcarboxamido)acetic acid;
2-((S)-5-acetamido-2′,4′-dioxo-2,3-dihydrospiro[indene-1,5′-ozazolidine]-3′-yl)-N-benzyl-N—((S)-1-cyclopropylethyl)acetamide;
2-((R)-5-acetamido-2′,4′-dioxo-2,3-dihydrospiro[indene-1,5′-ozazolidine]-3′-yl)-N-benzyl-N—((S)-1-cyclopropylethyl)acetamide;
N-benzyl-2-{5-[(methylcarbamoyl)amino]-3′,5′-dioxo-2,3-dihydrospiro[indene-1,2′-[1,4]ozazolidine]-4″-yl}-N-(pentan-3-yl)acetamide;
2-amino-N-(4′-{[benzyl(pentan-3-yl)carbamoyl]methyl}-3′,5′-dioxo-2,3-dihydrospiro[indene-1,2′-[1,4]ozazolidine]-5-yl)-2′-methylpropanamide;
2-{5-[(azetidin-3-ylmethyl)amino]-3′,5′-dioxo-2,3-dihydrospiro[indene-1,2′-[1,4]ozazolidine]-4′-yl}-N-benzyl-N-[(1S)-1-cyclopropylethyl]acetamide;
N-benzyl-N-[(1S)-1-cyclopropylethyl]-2-{5-[(1,3-dihydroxypropan-2-yl)oxy]-3′,5′-dioxo-2,3-dihydrospiro[indene-1,2′-[1,4]ozazolidine]-4′-yl}acetamide;
1-amino-N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]cyclobutane-1-carboxamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]azetidine-3-carboxamide;
4-{[4′-({benzyl[(1S)-1-cyclopropylethyl]carbamoyl}methyl)-3′,5′-dioxo-2,3-dihydrospiro[indene-1,2′-[1,4]ozazolidine]-5-ylmethyl]amino}benzoic acid;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-2,3-dihydroxypropanamide;
N-benzyl-2-{5-[(methylcarbamoyl)amino]-3′,5′-dioxo-2,3-dihydrospiro[indene-1,2′-[1,4]ozazolidine]-4′-yl}-N-(oxolan-3-yl)acetamide;
N-benzyl-N-[(1S)-1-cyclopropylethyl]-2-{5-[(1,1-dioxo-1λ⁶-thian-4-yl)amino]-3′,5′-dioxo-2,3-dihydrospiro[indene-1,2′-[1,4]ozazolidine]-4′-yl}acetamide;
2-{5-[(2R)-2-amino-2-cyclopropylacetamido]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-(cyclopropylmethyl)-2-{5-[(methylcarbamoyl)amino]-3′,5′-dioxo-2,3-dihydrospiro[indene-1,2′-[1,4]ozazolidine]-4′-yl}-N-[(2-methylphenyl)methyl]acetamide;
N-ethyl-2-{5-[(methylcarbamoyl)amino]-3′,5′-dioxo-2,3-dihydrospiro[indene-1,2′-[1,4]ozazolidine]-4′-yl}-N-[(2-methylphenyl)methyl]acetamide;
N-[(1S)-1-cyclopropylethyl]-2-{3′,5′-dioxo-5-[(pyrrolidin-3-yl)amino]-2,3-dihydrospiro[indene-1,2′-[1,4]ozazolidine]-4′-yl}-N-[(4-fluorophenyl)methyl]acetamide;
2-(5-{[(1-aminocyclopropyl)methyl]amino}-3′,5′-dioxo-2,3-dihydrospiro[indene-1,2′-[1,4]ozazolidine]-4′-yl)-N-[(1S)-1-cyclopropylethyl]-N-[(4-fluorophenyl)methyl]acetamide;
N-benzyl-2-{5-[(methylcarbamoyl)amino]-3′,5′-dioxo-2,3-dihydrospiro[indene-1,2′-[1,4]ozazolidine]-4′-yl}-N-(oxan-4-yl)acetamide;
N-benzyl-N-[(1R)-1-cyclopropyl-2,2,2-trifluoroethyl]-2-{5-[(methylcarbamoyl)amino]-3′,5′-dioxo-2,3-dihydrospiro[indene-1,2′-[1,4]ozazolidine]-4′-yl}acetamide;
N-benzyl-2-{5-[(methylcarbamoyl)amino]-3′,5′-dioxo-2,3-dihydrospiro[indene-1,2′-[1,4]ozazolidine]-4′-yl}-N-(oxan-3-yl)acetamide;
N-[4′-({benzyl[(1S)-1-cyclopropylethyl]carbamoyl}methyl)-3′,5′-dioxo-2,3-dihydrospiro[indene-3,2′-[1,4]ozazolidine]-5-yl]ethanediamide;
2-{5-[(azetidin-3-yl)amino]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-benzyl-N-[(1S)-1-cyclopropylethyl]-2-{3′,5′-dioxo-5-[(2-oxopyrrolidin-3-yl)amino]-2,3-dihydrospiro[indene-1,2′-[1,4]ozazolidine]-4′-yl}acetamide;
2-{[4′-({benzyl[(1S)-1-cyclopropylethyl]carbamoyl}methyl)-3′,5′-dioxo-2,3-dihydrospiro[indene-1,2′-[1,4]ozazolidine]-5-yl]carbamoyl}-2,2-dimethylacetic acid;
N-benzyl-N-[(1S)-1-cyclopropylethyl]-2-{5-[(2-hydroxyethyl)amino]-3′,5′-dioxo-2,3-dihydrospiro[indene-1,2′-[1,4]ozazolidine]-4′-yl}acetamide;
(2R)-2-amino-N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]propanamide;
{[4′-({benzyl[(1S)-1-cyclopropylethyl]carbamoyl}methyl)-3′,5′-dioxo-2,3-dihydrospiro[indene-1,2′-[1,4]ozazolidine]-5-yl]carbamoyl}formic acid;
(2S)-2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-hydroxypropanamide;
N-[(1S)-1-cyclopropylethyl]-2-{3′,5′-dioxo-5-[(piperidin-3-yl)amino]-2,3-dihydrospiro[indene-1,2′-[1,4]ozazolidine]-4′-yl}-N-[(4-fluorophenyl)methyl]acetamide;
N-benzyl-N-[(1S)-1-cyclopropylethyl]-2-{5-[(methylcarbamoyl)amino]-3′,5′-dioxo-2,3-dihydro(3,3-²H₂)spiro[indene-1,2′-[1,4]ozazolidine]-4′-yl}acetamide;
N-[(4-fluorophenyl)methyl]-2-{5-[(methylcarbamoyl)amino]-3′,5′-dioxo-2,3-dihydrospiro[indene-1,2′-[1,4]ozazolidine]-4′-yl}-N-[(2S)-1,1,1-trifluoropropan-2-yl]acetamide;
2-amino-N-[4′-({benzyl[(1R)-1-cyclopropyl-2,2,2-trifluoroethyl]carbamoyl}methyl)-3′,5′-dioxo-2,3-dihydrospiro[indene-1,2′-[1,4]ozazolidine]-5-yl]-2-methylpropanamide;
2-{5-[(methylcarbamoyl)amino]-3′,5′-dioxo-2,3-dihydrospiro[indene-1,2′-[1,4]ozazolidine]-4′-yl}-N-[(2-methylphenyl)methyl]-N-(2-methylpropyl)acetamide;
2-{5-[(4-aminooxolan-3-yl)amino]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-[(4-²H)benzyl]-N-[(1S)-1-cyclopropylethyl]-2-{5-[(methylcarbamoyl)amino]-3′,5′-dioxo-2,3-dihydrospiro[indene-1,2′-[1,4]ozazolidine]-4′-yl}acetamide;
N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-2-methyl-2-(methylamino)propanamide;
(2R)-2-amino-N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-benzyl-N-[(1S)-1-cyclopropylethyl]-2-[(1S)-5-[(methylcarbamoyl)amino]-3′,5′-dioxo-2,3-dihydrospiro[indene-1,2′-[1,4]ozazolidine]-4′-yl]acetamide;
N-benzyl-N-[(1S)-1-cyclopropylethyl]-2-[(1R)-5-[(methylcarbamoyl)amino]-3′,5′-dioxo-2,3-dihydrospiro[indene-1,2′-[1,4]ozazolidine]-4′-yl]acetamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-methylazetidine-3-carboxamide;
N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylazetidine-3-carboxamide;
2-{5-[(2R)-2-amino-2-cyclopropylacetamido]-3′,5′-dioxo-2,3-dihydrospiro[indene-1,2′-[1,4]ozazolidine]-4′-yl}-N-[(4-fluorophenyl)methyl]-N-[(2S)-1,1,1-trifluoropropan-2-yl]acetamide;
N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylazetidine-3-carboxamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-2-(dimethylamino)-2-methylpropanamide;
(2R)-2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-hydroxypropanamide;
2-{5-bromo-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
2-{5-[(3-aminocyclohexyl)amino]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-[1-(azetidin-3-yl)ethyl]-N-benzyl-2-{5-[(methylcarbamoyl)amino]-3′,5′-dioxo-2,3-dihydrospiro[indene-1,2′-[1,4]ozazolidine]-4′-yl}acetamide;
2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-2-methylpropanamide;
N-[(4-fluorophenyl)methyl]-N-[(2S)-1-methoxypropan-2-yl]-2-{5-[(methylcarbamoyl)amino]-3′,5′-dioxo-2,3-dihydrospiro[indene-1,2′-[1,4]ozazolidine]-4′-yl}acetamide;
3-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]oxetane-3-carboxamide;
2-amino-N-[(1S)-4′-({[(4-bromophenyl)methyl][(1S)-1-cyclopropylethyl]carbamoyl}methyl)-3′,5′-dioxo-2,3-dihydrospiro[indene-1,2′-[1,4]ozazolidine]-5-yl]-2-methylpropanamide;
2-amino-N-[(1R)-4′-({[(4-bromophenyl)methyl][(1S)-1-cyclopropylethyl]carbamoyl}methyl)-3′,5′-dioxo-2,3-dihydrospiro[indene-1,2′-[1,4]ozazolidine]-5-yl]-2-methylpropanamide;
2-{5-[(2R)-2-amino-2-(oxetan-3-yl)acetamido]-3′,5′-dioxo-2,3-dihydrospiro[indene-1,2′-[1,4]ozazolidine]-4′-yl}-N-benzyl-N-[(1S)-1-cyclopropylethyl]acetamide;
1-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3-difluorocyclobutane-1-carboxamide;
2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1S)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1R)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]-N-[(2S)-1,1,1-trifluoropropan-2-yl]acetamide;
N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]-N-[(2S)-1,1,1-trifluoropropan-2-yl]acetamide;
N-[(1S)-1-cyclopropylethyl]-N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]acetamide;
N-[(1S)-1-cyclopropylethyl]-N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]acetamide;
(2S)-2-amino-N-[(1S)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1S)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2S)-2-amino-N-[(1R)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1R)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
N-benzyl-N-[(1R)-1-cyclopropyl-2,2,2-trifluoroethyl]-2-[(1S)-5-[(methylcarbamoyl)amino]-3′,5′-dioxo-2,3-dihydrospiro[indene-1,2′-[1,4]ozazolidine]-4′-yl]acetamide;
N-benzyl-N-[(1R)-1-cyclopropyl-2,2,2-trifluoroethyl]-2-[(1R)-5-[(methylcarbamoyl)amino]-3′,5′-dioxo-2,3-dihydrospiro[indene-1,2′-[1,4]ozazolidine]-4′-yl]acetamide;
(2R)-2-amino-N-[(1S)-4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1R)-4′-({[(4-fluorophenyl)methyl ][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1S)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1R)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(1R)-1-cyclopropyl-2,2,2-trifluoroethyl]-N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]acetamide;
N-[(1R)-1-cyclopropyl-2,2,2-trifluoroethyl]-N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]acetamide;
N-[(1S)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
N-[(1R)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
(2R)-2-amino-N-[4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(1S)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
N-[(1R)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
(2S)-2-amino-N-[4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
(2S)-2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]azetidine-3-carboxamide;
N-benzyl-N-[1-(1-methylazetidin-3-yl)ethyl]-2-{5-[(methylcarbamoyl)amino]-3′,5′-dioxo-2,3-dihydrospiro[indene-1,2′-[1,4]ozazolidine]-4′-yl}acetamide;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1R)-3′-(2-{[(1R)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-benzyl-2-[(1R)-5-bromo-2′,4′-dioxo-2,3-dihydro-3′H-spiro[indene-1,5′-[1,3]ozazolidin]-3′-yl]-N-[(1S)-1-cyclopropylethyl]acetamide;
2-[(1S)-5-amino-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-[(1R)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-[(1S)-3′-(2-{[(1R)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-[(1R)-1-cyclopropylethyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1R)-1-cyclopropylethyl]-N-(4-fluorobenzyl)-2-{(1S)-5-[(methylcarbamoyl)amino]-2′,4′-dioxo-2,3-dihydro-3′H-spiro[indene-1,5′-[1,3]ozazolidin]-3′-yl}acetamide;
N-[(1R)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-[(1S)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]azetidine-1-carboxylate;
2-[(1R)-5-(6-aminopyridin-3-yl)-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-{(1R)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-[(1S)-1-cyclopropylethyl]-N-(4-fluorobenzyl)acetamide;
N-[(1S)-1-cyclopropylethyl]-2-[(1R)-2′,4′-dioxo-5-(pyridin-3-yl)-2,3-dihydro-3′H-spiro[indene-1,5′-[1,3]ozazolidin]-3′-yl]-N-(4-fluorobenzyl)acetamide;
2-[(1R)-5-(azetidin-3-yl)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
2-[(1R)-5-(1-acetylazetidin-3-yl)-2′,4′-dioxo-2,3-dihydro-3′H-spiro[indene-1,5′-[1,3]ozazolidin]-3′-yl]-N-benzyl-N-[(3S)-1-cyclopropylethyl]acetamide;
N-benzyl-N-[(1S)-1-cyclopropylethyl]-2-[(1R)-5-(1-methylazetidin-3-yl)-2′,4′-dioxo-2,3-dihydro-3′H-spiro[indene-1,5′-[1,3]ozazolidin]-3′-yl]acetamide;
3-[(1R)-3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]-N-methylazetidine-1-carboxamide;
N-benzyl-2-{(1R)-5-[1-(cyclopropylcarbonyl)azetidin-3-yl]-2′,4′-dioxo-2,3-dihydro-3′H-spiro[indene-1,5′-[1,3]ozazolidin]-3′-yl}-N-[(1S)-1-cyclopropylethyl]acetamide;
N-benzyl-N-[(1S)-1-cyclopropylethyl]-2-{(1R)-5-[1-(2-hydroxy-2-methylpropanoyl)azetidin-3-yl]-2′,4′-dioxo-2,3-dihydro-3′H-spiro[indene-1,5′-[1,3]ozazolidin]-3′-yl}acetamide;
N-benzyl-N-[(1S)-1-cyclopropylethyl]-2-{(1R)-5-[1-(methylsulfonyl)azetidin-3-yl]-2′,4′-dioxo-2,3-dihydro-3′H-spiro[indene-1,5′-[1,3]ozazolidin]-3′-yl}acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]cyclobutanecarboxamide;
3-ethoxy-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1, 3]ozazolidin]-5-yl]-5-oxo-D-prolinamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-5-oxo-L-prolinamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanimide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]cyclopropanecarboxamide;
N-(4-fluorobenzyl)-2-{(1R)-5-[(methoxyacetyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(1R)-5-[(ethoxyacetyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-3-methylbutanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylpropanamide;
2-[(1R)-5-(acetylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-{[(2-methoxyethyl)(methyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[(dimethylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-{[methyl(2-methylpropyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(cyclopropylmethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(cyclobutylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-({[(2R)-1-hydroxy-3-methylbutan-2-yl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-({[(2S)-tetrahydrofuran-2-ylmethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-({[(2R)-tetrahydrofuran-2-ylmethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-({[2-(propan-2-yloxy)ethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(cyclopropylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-{[ethyl(methyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(diethylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyrrolidine-1-carboxamide;
2-[(1R)-5-({[2-(dimethylamino)ethyl](methyl)carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N²-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-L-leucinamide;
N-(4-fluorobenzyl)-2-[(1R)-5-{[(2-hydroxy ethyl)(propyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(2-methoxyethyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(3-hydroxypropyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-({[(2S)-1-hydroxy-3-methylbutan-2-yl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[methyl(propan-2-yl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(2-cyanoethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-{[methyl(propyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-[(1S)-1-cyclopropylethyl]-2-[(1R)-2′,4′-dioxo-5-(pyridin-4-yl)-2,3-dihydro-3′H-spiro[indene-1,5′-[1,3]ozazolidin]-3′-yl]-N-(4-fluorobenzyl)acetamide;
N-[(1S)-1-cyclopropylethyl]-N-(4-fluorobenzyl)-2-[(1R)-5-(6-hydroxypyridazin-3-yl)-2′,4′-dioxo-2,3-dihydro-3′H-spiro[indene-1,5′-[1,3]ozazolidin]-3′-yl]acetamide;
N-[(1S)-1-cyclopropylethyl]-2-[(1R)-2′,4′-dioxo-5-(1H-pyrazol-4-yl)-2,3-dihydro-3′H-spiro[indene-1,5′-[1,3]ozazolidin]-3′-yl]-N-(4-fluorobenzyl)acetamide;
2-{(1R)-2′,4′-dioxo-5-[(tetrahydrofuran-2-ylmethyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[(cyclopentylmethyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-(2,2-dimethylbutyl)amino-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-2′,4′-dioxo-5-[(tetrahydrofuran-3-ylmethyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-{(1R)-5-[(2-methylpropyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-[(1S)-1-cyclopropylethyl]-2-[(1R)-2′,4′-dioxo-5-(pyridin-2-yl)-2,3-dihydro-3′H-spiro[indene-1,5′-[1,3]ozazolidin]-3′-yl]-N-(4-fluorobenzyl)acetamide;
N-[(1S)-1-cyclopropylethyl]-N-(3-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(1R)-5-(carbamoylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-((R)-5-(3-(N-methylsulfamoyl)ureido)-2′,4′-dioxo-2,3-dihydrospiro[indene-1,5′-ozazolidine]-3′-yl)-N—((S)-1,1,1-trifluoropropan-2-yl)acetamide
methyl {[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}sulfamate;
2-((R)-5-(3-(N-(cyclopropylmethyl)sulfamoyl)ureido)-2′,4′-dioxo-2,3-dihydrospiro[indene-1,5′-ozazolidine]-3′-yl)-N-(4-fluorobenzyl)-N—((S)-1,1,1-trifluoropropan-2-yl)acetamide
(1R)-3′-(2-{benzyl[(1S)-1-cyclopropylethyl]amino}-2-oxoethyl)-N-(2-methylpropyl)-2′,4′-dioxo-2,3-dihydrospiro[indene-1,5′-[1,3]ozazolidine]-5-carboxamide;
N-[(1S)-1-cyclopropylethyl]-N-(2-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,3-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-chloro-4-fluorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3-chloro-4-fluorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-[4-(trifluoromethyl)benzyl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1-methyl-1H-pyrazole-4-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1-methyl-1H-pyrazole-3-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-4-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-2-carboxamide;
2-(5-bromo-4-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
2-(5-bromo-6-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
(1R)-3′-(2-{benzyl[(1S)-1-cyclopropylethyl]amino}-2-oxoethyl)-N-[(2R)-1-hydroxy-3-methylbutan-2-yl]-2′,4′-dioxo-2,3-dihydrospiro[indene-1,5′-[1,3]ozazolidine]-5-carboxamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-[(5-methyl-1,3,4-thiadiazol-2-yl)methyl]acetamide;
2-(5-amino-4-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{4-fluoro-5-[(methylcarbamoyl)amino]-2′,4′-dioxo-2,3-dihydro-3′H-spiro[indene-1,5′-[1,3]ozazolidin]-3′-yl}acetamide;
N-(4-cyanobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3-methylbenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,4-dichlorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-methoxybenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3-methoxybenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-methylbenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-[(1R)-5-{[(1-methyl-1H-pyrazol-4-yl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-2′,4′-dioxo-5-[(phenylcarbamoyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-2′,4′-dioxo-5-[(pyridin-3-ylcarbamoyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-(5-amino-6-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{6-fluoro-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetamide;
N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(dimethylamino)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(4-methylpyrimidin-5-yl)-2′,4′-dioxo-2,3-dihydro-3′H-spiro[indene-1,5′-[1,3]ozazolidin]-3′-yl]-N-[(2S)-1,1,1-trifluoropropan-2′-yl]acetamide;
2-{(1R)-5-[5-(difluoromethyl)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(6-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1R)-5-[1-(2-methylpropyl)-1H-pyrazol-4-yl]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(1R)-5-[4-(difluoromethyl)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(3-fluoropyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(1,3-thiazol-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-N-methylbenzamide;
2-{(1R)-5-[2-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(5-cyanopyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(1-methyl-1H-1,2,4-triazol-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(5-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-(6-cyanopyridin-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2 yl]acetamide;
N-(4-fluorobenzyl)-2-[(1R)-5-(5-fluoro-3-methylpyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(5-methyl-1,3,4-thiadiazol-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(4-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(6-methoxypyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[2-(dimethylamino)pyrimidin-5-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(acetylamino)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(1-methyl-1H-1,2,4-triazol-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-cyclopropyl-5-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]furan-2-carboxamide;
N-(4-fluorobenzyl)-2-[(1R)-5-(5-fluoropyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
4-fluoro-3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
5-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-N-methylpyridine-2-carboxamide;
6-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-3-carboxamide;
N-(4-fluorobenzyl)-2-[(1R)-5-(2-methoxypyrimidin-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
6-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-2-carboxamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]thiophene-3-carboxamide;
2-{(1R)-5-[4-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(1,3-dimethyl-2,4-dioxo-1,2,3,4-tetrahydropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4-cyanopyridin-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-4-carboxamide;
2-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
2-{(1R)-5-[3-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(6-cyano-5-methoxypyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(1-methyl-1H-pyrazol-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(1,3,5-trimethyl-1H-pyrazol-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(3,4-difluorophenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2,5-difluoro-4-methoxyphenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyrimidin-5-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-2′,4′-dioxo-5-[5-(trifluoromethyl)pyridin-3-yl]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-2′,4′-dioxo-5-(1,3-thiazol-2-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2-cyanopyridin-4-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyrazin-2-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[4-(carbamoylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(2-cyanopyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(6-cyano-5-fluoropyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
2-[(1R)-5-{[(4-aminophenyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(pyrimidin-5-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(pyrimidin-2-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-triazol-5-ylmethyl)acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]prolinamide;
N²-(4-fluorobenzyl)-N²-({(1R)-5-[(methylcarbamoyl)amino]-2′,4′-dioxo-2,3-dihydro-3′H-spiro[indene-1,5′-[1,3]ozazolidin]-3′-yl}acetyl)-L-alaninamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1H-pyrazole-5-carboxamide;
N-(4-fluorobenzyl)-2-[(1R)-5-{[(methylsulfamoyl)acetyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(methylsulfonyl)acetyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(dimethylsulfamoyl)acetyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-2′,4′-dioxo-5-[(sulfamoylacetyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidine-1-carboxamide
N-benzyl-N-ethyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
methyl N′-cyano-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamimidothioate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanamide;
N-(4-fluorobenzyl)-2-{(1R)-5-[(methylcarbamothioyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(tetrahydro-2H-pyran-4-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-oxazol-4-ylmethyl)acetamide;
2-[(1R)-5-(5-cyanothiophen-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4-cyano-3-fluorophenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(5-cyanothiophen-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(N″-cyano-N′-methylcarbamimidamido)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4,6-difluoropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[2-(acetylamino)-5-methylpyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(5-fluoropyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(furan-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(2-oxo-2,3-dihydro-1H-benzimidazol-5-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(4-methylpyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-2′,4′-dioxo-5-[1-(tetrahydro-2H-pyran-2-yl)-1H-pyrazol-3-yl]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(5-fluoro-6-methylpyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[6-(difluoromethyl)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(difluoromethyl)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(furan-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[1-(ethoxymethyl)-1H-imidazol-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(5-cyano-1,2-dimethyl-6-oxo-1,6-dihydropyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]thiophene-2-carboxamide;
2-[(1R)-5-(2,6-dioxo-1,2,5,6-tetrahydropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2,4-dimethyl-1,3-thiazol-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[1-(cyanomethyl)-3,5-dimethyl-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[3-(carbamoylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-[3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)-2,2-dimethylpropyl]propanamide;
(1R)-3′-(2-{benzyl[(1S)-1-cyclopropylethyl]amino}-2-oxoethyl)-N-methyl-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(propan-2-yl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(2-methoxyethyl)-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(tetrahydrofuran-2-ylmethyl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(tetrahydrofuran-3-ylmethyl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(cyanomethyl)-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(3-hydroxypropyl)-2′,4′-dioxo-2,3-dihydrospiro[indene-1,5′-[1,3]ozazolidine]-5-carboxamide;
3′-{2-[benzyl(1-cyclopropylethyl)amino]-2-oxoethyl}-N-(1-hydroxy-3-methylbutan-2-yl)-2′,4′-dioxo-2,3-dihydrospiro[indene-1,5′-[1,3]ozazolidine]-5-carboxamide;
N-(1-amino-4-methyl-1-oxopentan-2-yl)-3′-{2-[benzyl(1-cyclopropylethyl)amino]-2-oxoethyl}-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-[2-(diethylamino)ethyl]-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-[3-(1H-imidazol-1-yl)propyl]-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-[3-(2-oxopyrrolidin-1-yl)propyl]-2,3-dihydrospiro[indene-1,5′-[1,3]ozazolidine]-5-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]tetrahydropyrimidine-1(2H)-carboxamide;
3-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]tetrahydropyrimidine-1(2H)-carboxamide;
N-(4-fluorobenzyl)-2-{(1R)-5-[1-(2-hydroxyethyl)-1H-pyrazol-4-yl]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-phenylacetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N,N-dibenzyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
(2R)-3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-hydroxy-2-methylpropanamide;
(2S)-3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-hydroxy-2-methylpropanamide;
2-[(1R)-2′,4′-dioxo-5-({[3-(pyrrolidin-1-ylmethyl)phenyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-({[3-(morpholin-4-ylmethyl)phenyl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N³-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-N-methyl-beta-alaninamide;
N-[3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)phenyl]propanamide;
N-(2-chlorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)acetamide;
2-[(1R)-5-(4,5-dihydro-1H-imidazol-2-ylamino)-2′,4′-dioxo-2,3-dihydro-3′H-spiro[indene-1,3]ozazolidin]-3′-yl]-N-(4-fluorobenzyl)-N-(2S)-1,1,1-trifluoropropan-2-yl]acetamide;
2-[(1R)-5-({[3-(acetylamino)propyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-({[2-(acetylamino)ethyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
tert-butyl [3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)-2,2-dimethylpropyl]carbamate;
N-[2-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)ethyl]propanamide;
2-[(1R)-5-{[(3-amino-2,2-dimethylpropyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-({[3-(acetylamino)phenyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
methyl 2-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]amino}-4,5-dihydro-1H-imidazole-1-carboxylate;
N-cyclohexyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-cyclopentyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-[1-(furan-2-yl)ethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-beta-alanine;
N-cyclopropyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl N-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-beta-alaninate;
N-(3,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(4-fluorobenzyl)-N-(3-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(1S)-5-amino-2′,4′-dioxo-2,3-dihydro-3′H-spiro[indene-1,5′-[1,3]ozazolidin]-3′-yl]-N-[(1R)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
ethyl ({(3R)-3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(3R)-piperidin-3-yl]acetamide;
ethyl ({(3R)-3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]piperidin-1-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(methylsulfonyl)piperidin-4-yl]acetamide;
N-(4-fluorobenzyl)-2-{6-fluoro-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-(5-amino-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1-phenylethyl)acetamide;
2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)-N-[2-(trifluoromethyl)benzyl]acetamide;
2-(5-bromo-6-fluoro-2′,4′-dioxo-2,3-dihydro-3′H-spiro[indene-1,5′-[1,3]ozazolidin]-3′-yl) -N-(4-fluorobenzyl)-N-[(2S)-1,1,1-trifluoropropan-2-yl]acetamide;
N-benzyl-N-(cyanomethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-(2-cyanoethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(methylsulfonyl)propan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(1-methyl-1H-pyrazol-4-yl)methyl]acetamide;
N-[(1S)-1-cyclopropylethyl]-N-(4-fluorobenzyl)-2-{4-methoxy-5-[(methylcarbamoyl)amino]-2′,4′-dioxo-2,3-dihydro-3′H-spiro[indene-1,5′-[1,3]ozazolidin]-3′-yl}acetamide;
2-[5-(acetylamino)-4-methoxy-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-[(1R)-5-({[3-(acetylamino)-2,2-dimethylpropyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclopentyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(pyridin-3-yl)acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1-methyl-1H-pyrazol-4-yl)acetamide;
N-benzyl-N-(2,2-difluorocyclopentyl)-2-{5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-(2-methylcyclopropyl)acetamide;
N-(2,2-dimethylcyclopentyl)-N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-(2-methylcyclopentyl)acetamide;
2-(5-{[(2-cyanoethyl)carbamoyl]amino}-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
methyl [(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamate;
4-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)butanoic acid;
N²-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylalaninamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylalaninamide;
N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
N-cyclopentyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-oxazol-4-ylmethyl)acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-[1-(pyridin-2-yl)ethyl]acetamide;
N-(4-chlorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3,5-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-(5-{[(cyanomethyl)carbamoyl]amino}-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
2-[(1R)-5-{[(cyanomethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclohexyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-pyrrolidin-3-yl]acetamide;
N-(4-fluorobenzyl)-N-(trans-3-hydroxycyclobutyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-1-(methylsulfonyl)pyrrolidin-3-yl]acetamide;
ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidin-1-yl}sulfonyl)carbamate;
N-(1-cyanopropan-2-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
5-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)pentanoic acid;
N-[(1S)-1-cyclopropyl-2,2,2-trifluoroethyl]-N-(4-fluorobenzyl)-2-[4-fluoro-5-(1-methyl-1H-pyrazol-4-yl)-2′,4′-dioxo-2,3-dihydro-3′H-spiro[indene-1,5′-[1,3]ozazolidin]-3′-yl]acetamide;
N-(4-fluorobenzyl)-2-[6-fluoro-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-[(3R)-1-acetylpyrrolidin-3-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-1-(2-sulfamoylethyl)pyrrolidin-3-yl]acetamide;
N-(4-fluorobenzyl)-N-(trans-4-hydroxycyclohexyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
methyl (1R,3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-3-(methylsulfonyl)tetrahydropyrimidine-1(2H)-carboxamide;
N²-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]glycinamide;
2-{(1R)-5-[(methylcarbamoyl)amino]-2′,4′-dioxo-2,3-dihydro-3′H-spiro[indene-1,5′-[1,3]ozazolidin]-3′-yl}-N-[1-(2-methylphenyl)ethyl]-N-(2-methylpropyl)acetamide;
N-(2-chlorobenzyl)-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)acetamide;
N-(4-fluorobenzyl)-N-(4-methoxyphenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(4-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(2-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(trifluoromethyl)cyclopropyl]acetamide;
2-[(1R)-5-(3,4-dihydro-2H-pyrrol-5-ylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(3,4-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(2,5-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3-chlorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{5-[1-(2-amino-2-oxoethyl)-1H-pyrazol-4-yl]-6-fluoro-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(3R)-pyrrolidin-3-yl]acetamide;
ethyl ({(3R)-3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]pyrrolidin-1-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(piperidin-4-yl)acetamide;
ethyl ({4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
tert-butyl 3-[(4-fluorobenzyl)({(1R)-5-(methylcarbamoyl)amino]-2′,4′-dioxo-2,3-dihydro-3′H-spiro[indene-1,5′-[1,3]ozazolidin]-3′-yl}acetyl)amino]azetidine-1-carboxylate;
N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}-N-[2-(trifluoromethyl)benzyl]acetamide;
N-(2-chloro-4-fluorobenzyl)-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(3-methoxyphenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-(pyridin-4-yl)acetamide;
N-(4-fluorobenzyl)-2-(6-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl)-N-[(2S)-1,1,1-trifluoropropan-2-yl]acetamide;
methyl (1S,3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(tetrahydro-2H-pyran-4-yl)acetamide;
N-(1,1-dioxidotetrahydro-2H-thiopyran-4-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2,2-difluorocyclopentyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-cyclohexyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-(4-fluorobenzyl)-N-{1-[2-(methylamino)-2-oxoethyl]-1H-pyrazol-4-yl}-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[1-(cyanomethyl)-1H-pyrazol-4-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl 3-{1-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]ethyl}azetidine-1-carboxylate;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylic acid;
ethyl ({(3S)-3-[(4-fluorobenzyl){[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
ethyl ({(3S)-3-[{[(1R)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}(4-fluorobenzyl)amino]pyrrolidin-1-yl}sulfonyl)carbamate;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}acetate;
ethyl ({(3R)-3-[(4-fluorobenzyl){[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]piperidin-1-yl}sulfonyl)carbamate;
ethyl ({(3R)-3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]piperidin-1-yl}sulfonyl)carbamate;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}acetic acid;
tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
tert-butyl (3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-piperidin-3-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3S)-piperidin-3-yl]acetamide;
N-[4-amino-3-(hydroxymethyl)butan-2-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({(3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzamide;
tert-butyl 2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(pyrrolidin-2-ylmethyl)acetamide;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexylidene}acetate;
N-ethyl-4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxamide;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexylidene}acetic acid;
N-(4-fluorobenzyl)-2-[(1R)-5-(oxetan-3-ylamino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzamide;
N-[(1-acetylpyrrolidin-2-yl)methyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{[1-(methylsulfonyl)pyrrolidin-2-yl]methyl}acetamide;
2-{[(4-fluorobenzyl) ({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}-N-methylpyrrolidine-1-carboxamide;
N-(3,4-difluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{[1-(methylsulfamoyl)pyrrolidin-2-yl]methyl}acetamide;
N-(cyclopropylmethyl)-N-(2,3-dihydro-1H-indene-1-yl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexyl}acetic acid;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-3-methylazetidin-1-yl}sulfonyl)carbamate;
N-(3-cyanophenyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
tert-butyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}carbamate;
N-(3-aminocyclobutyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[3-(acetylamino)cyclobutyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{3-[(methylsulfonyl)amino]cyclobutyl}acetamide;
2,2′-({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}imino)diacetic acid;
N-{3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}glycine;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxylate;
methyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}carbamate;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}sulfamoyl)carbamate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxylic acid;
ethyl ({4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]piperidin-1-yl}sulfonyl)carbamate;
2-(5-chloro-4-cyano-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
benzyl 3-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}azetidine-1-carboxylate;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoate;
ethyl ({3,3-difluoro-4-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
ethyl ({3,3-difluoro-4-[(4-fluorobenzyl){[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
N-(4-fluorobenzyl)-N-(3-methoxycyclobutyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-N-methylcyclobutanecarboxamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxamide;
2-(5-amino-4-cyano-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetic acid;
(2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidin-1-yl)acetic acid;
methyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetate;
{3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetic acid;
tert-butyl {4-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]cyclohexylidene}acetate;
tert-butyl {4-[(4-fluorobenzyl){[(1S)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetate;
{4-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetic acid;
N-(azetidin-3-ylmethyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl {trans-4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexyl}carbamate;
N-(trans-4-aminocyclohexyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{trans-4-[(methylsulfonyl)amino]cyclohexyl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(trans-4-{[(trifluoromethyl)sulfonyl]amino}cyclohexyl)acetamide;
tert-butyl 6-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-2-azaspiro[3.3]heptane-2-carboxylate;
N-(2-azaspiro[3.3]hept-6-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1-acetylazetidin-3-ylmethyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-[(2S)-1,1,1-trifluoropropan-2-yl]-2-(2′,3,4′-trioxo-1,2,3,4,8,9-hexahydro-3′H-spiro[cyclopenta[f]quinazoline-7,5′-[1,3]ozazolidin]-3′-yl)acetamide;
ethyl [(3-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}azetidin-1-yl)sulfonyl]carbamate;
ethyl [(2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidin-1-yl)sulfonyl]carbamate;
ethyl ({6-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-2-azaspiro[3.3]hept-2-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(3-{[(trifluoromethyl)sulfonyl]amino}cyclobutyl)acetamide;
tert-butyl 3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]benzoate;
tert-butyl 3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]benzoate;
3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]benzoic acid;
N-(4-fluorobenzyl)-2-[(1S)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(azetidin-3-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]azetidin-1-yl}sulfonyl)carbamate;
2-[(1S)-5-bromo-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1S)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-D-valinamide;
N-benzyl-N-[(1S)-1-cyclopropylethyl]-2-[2′,4′-dioxo-5-(2H-tetrazol-5-yl)-2,3-dihydro-3′H-spiro[indene-1,5′-[1,3]ozazolidin]-3′-yl]acetamide;
2,2′-({[3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]carbonyl}imino)diacetic acid;
N-(4-fluorobenzyl)-2-[(1S)-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
methyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanoate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanoic acid;
2-{(1S)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1S)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyridin-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(pyridin-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(pyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(6-oxo-1,6-dihydropyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(6-oxo-1,6-dihydropyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-phenyl-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-phenyl-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclohexyl-N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-(tetrahydro-2H-pyran-4-yl)acetamide;
3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclobutanecarboxylic acid;
4-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexanecarboxylic acid;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-{trans-4-[(methylsulfonyl)amino]cyclohexyl}acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-(3-{[(trifluoromethyl)sulfonyl]amino}cyclobutyl)acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-[1-(methylsulfonyl)piperidin-4-yl]acetamide;
2,2′-({3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclobutyl}imino)diacetic acid;
N-(4-fluorobenzyl)-2-[(1R)-5-(1,2-oxazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1S)-5-(1,2-oxazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide; and pharmaceutically acceptable salts thereof.

Still another embodiment pertains to compounds of Formula (XIIIb), selected from the group consisting of;
N-benzyl-2-(5′-bromo-2,5-dioxo-2′,3′-dihydrospiro[imidazolidine-4,1′-indene]-1-yl)-N-(1-cyclopropylethyl)acetamide;
2-(5′-bromo-2,5-dioxo-2′,3′-dihydrospiro[imidazolidine-4,1′-indene]-1-yl)-N-(1-cyclopropylethyl)-N-(furan-2-ylmethyl)acetamide;
N-benzyl-N-(1-cyclopropylethyl)-2-(5′-fluoro-2,5-dioxo-2′,3′-dihydrospiro[imidazolidine-4,1′-indene]-1-yl)acetamide;
N-benzyl-N-(1-cyclopropylethyl)-2-(5′-methoxy-2,5-dioxo-2′,3′-dihydrospiro[imidazolidine-4,1′-indene]-1-yl)acetamide;
N-benzyl-2-(5′-cyano-2,5-dioxo-2′,3′-dihydrospiro[imidazolidine-4,1′-indene]-1-yl)-N-(1-cyclopropylethyl)acetamide;
N-benzyl-2-(5′-bromo-2,5-dioxo-2′,3′-dihydrospiro[imidazolidine-4,1′-indene]-1-yl)-N-(1-cyclobutylethyl)acetamide;
2-(5′-bromo-2,5-dioxo-2′,3′-dihydrospiro[imidazolidine-4,1′-indene]-1-yl)-N-(1-cyclopropylethyl)-N-(4-fluorobenzyl)acetamide;
1-(2-(benzyl(1-cyclopropylethyl)amino)-2-oxoethyl)-2,5-dioxo-2′,3′-dihydrospiro[imidazolidine-4,1′-indene]-5′-carboxamide;
N-benzyl-N-(1-cyclopropylethyl)-2-(5′-(methylthio)-2,3-dioxo-2′,3′-dihydrospiro[imidazolidine-4,1′-indene]-1-yl)acetamide;
N-benzyl-N-(1-cyclopropylethyl)-2-(5′-(hydroxy methyl)-2,5-dioxo-2′,3′-dihydrospiro[imidazolidine-4,1′-indene]-1-yl)acetamide;
N-benzyl-N-(1-cyclopropylethyl)-2-(5′-((difluoromethoxy)methyl)-2,5-dioxo-2′,3′-dihydrospiro[imidazolidine-4,1′-indene]-1-yl)acetamide;
N-benzyl-2-(5′-cyclopropyl-2,5-dioxo-2′,3′-dihydrospiro[imidazolidine-4,1′-indene]-1-yl)-N-(1-cyclopropylethyl)acetamide;
N-benzyl-N-(1-cyclobutylethyl)-2-(5′-(difluoromethoxy)-2,5-dioxo-2′,3′-dihydrospiro[imidazolidine-4,1′-indene]-1-yl)acetamide;
2-(5′-acetamido-2,5-dioxo-2′,3′-dihydrospiro[imidazolidine-4,1′-indene]-1-yl)-N-benzyl-N-(1-cyclopropylethyl)acetamide;
N-benzyl-N-(1-cyclopropylethyl)-2-(5′-methyl-2,5-dioxo-2′,3′-dihydrospiro[imidazolidine-4,1′-indene]-1-yl)acetamide;
1-(2-(benzyl((S)-1-cyclopropylethyl)amino)-2-oxoethyl)-N-(cyanomethyl)-2,5-dioxo-2′,3′-dihydrospiro[imidazolidine-4,1′-indene]-5′-carboxamide;
N-benzyl-N-(1-cyclopropylethyl)-2-(5′-ethynyl-2,5-dioxo-2′,3′-dihydrospiro[imidazolidine-4,1′-indene]-1-yl)acetamide;
N-benzyl-N-(1-cyclopropylethyl)-2-(5′-(3-hydroxyprop-1-ynyl)-2,5-dioxo-2′,3′-dihydrospiro[imidazolidine-4,1′-indene]-1-yl)acetamide;
N-benzyl-N-(1-cyclopropylethyl)-2-(2,5-dioxo-5′-(1H-pyrazol-5-yl)-2′,3′-dihydrospiro[imidazolidine-4,1′-indene]-1-yl)acetamide;
N-benzyl-N-(1-cyclopropylethyl)-2-(2,5-dioxo-5′-(1H-1,2,3-triazol-4-yl)-2′,3′-dihydrospiro[imidazolidine-4,1′-indene]-1-yl)acetamide;
2-(5′-(1H-imidazol-2-yl)-2,5-dioxo-2′,3′-dihydrospiro[imidazolidine-4,1′-indene]-1-yl)-N-benzyl-N-(1-cyclopropylethyl)acetamide;
N-benzyl-N-(1-cyclopropylethyl)-2-(2,5-dioxo-5′-(1H-1,2,3-triazol-1-yl)-2′,3′-dihydrospiro[imidazolidine-4,1′-indene]-1-yl)acetamide;
N-benzyl-N-(1-cyclopropylethyl)-2-(2,5-dioxo-5′-(2H-1,2,3-triazol-2-yl)-2′,3′-dihydrospiro[imidazolidine-4,1′-indene]-1-yl)acetamide;
N-benzyl-N-(1-cyclopropylethyl)-2-(5′-(methoxymethyl)-2,5-dioxo-2′,3′-dihydrospiro[imidazolidine-4,1′-indene]-1-yl)acetamide;
N-benzyl-2-(5′-(2-cyanoacetamido)-2,5-dioxo-2′,3′-dihydrospiro[imidazolidine-4,1′-indene]-1-yl)-N—((S)-1-cyclopropylethyl)acetamide;
methyl 1-(2-(benzyl((S)-1-cyclopropylethyl)amino)-2-oxoethyl)-2,5-dioxo-2′,3′-dihydrospiro[imidazolidine-4,1′-indene]-5′-ylcarbamate;
N-benzyl-N—((S)-1-cyclopropylethyl)-2-(5′-(3-methylureido)-2,5-dioxo-2′,3′-dihydrospiro[imidazolidine-4,1′-indene]-1 yl)acetamide;
N-benzyl-N—((S)-1-cyclopropylethyl)-2-(5′-(3-methyl-3-(methylcarbamoyl)ureido)-2,5-dioxo-2′,3′-dihydrospiro[imidazolidine-4,1′-indene]-1-yl)acetamide;
N-benzyl-N—((S)-1-cyclopropylethyl)-2-(5′-(3-hydroxy-3-methylbut-1-ynyl)-2,5-dioxo-2′,3′-dihydrospiro[imidazolidine-4,1′-indene]-1-yl)acetamide;
N-((1-(2-amino-2-oxoethyl)-1H-1,2,3-triazol-4-yl)methyl)-N—((S)-1-cyclobutylethyl)-2-((S)-2,5-dioxo-2′,3′-dihydrospiro[imidazolidine-4,1′-indene]-1-yl)acetamide;
(S)—N-benzyl-N-(1-cyclopropylethyl)-2-(2,5-dioxo-5′-(6-oxo-1,6-dihydropyridin-3-yl)-2′,3′-dihydrospiro[imidazolidine-4,1′-indene]-1-yl)acetamide;
N-benzyl-N—((S)-1-cyclopropylethyl)-2-((S)-2,5-dioxo-5′-(1H-pyrazol-3-yl)-2′,3′-dihydrospiro[imidazolidine-4,1′-indene]-1-yl)acetamide;
N-benzyl-N—((S)-1-cyclopropylethyl)-2-(5′-(methylsulfonamido)-2,5-dioxo-2′,3′-dihydrospiro[imidazolidine-4,1′-indene]-1-yl)acetamide;
N-benzyl-2-(5′-(2-cyanoacetamido)-2,5-dioxo-2′,3′-dihydrospiro[imidazolidine-4,1′-indene]-1-yl)-N—((S)-1-cyclopropyl ethyl)acetamide;
N-benzyl-N—((S)-1-cyclopropylethyl)-2-(2,5-dioxo-5′-(2H-tetrazol-5-yl)-2′,3′-dihydrospiro[imidazolidine-4,1′-indene]-1-yl)acetamide;
N-benzyl-N—((S)-1-cyclopropylethyl)-2-((S)-2,5-dioxo-5′-(6-oxo-1,6-dihydropyridin-3-yl)-2′,3′-dihydrospiro[imidazolidine-4,1′-indene]-1-yl)acetamide;
N-benzyl-N—((S)-1-cyclopropylethyl)-2-((S)-5′-(3-hydroxyprop-1-ynyl)-2,5-dioxo-2′,3′-dihydrospiro[imidazolidine-4,1′-indene]-1-yl)acetamide;
N-benzyl-N—((S)-1-cyclopropylethyl)-2-((S)-2,5-dioxo-5′-(2H-1,2,3-triazol-4-yl)-2′,3′-dihydrospiro[imidazolidine-4,1′-indene]-1-yl)acetamide;
N-benzyl-N—((S)-1-cyclopropylethyl)-2-(5′-(3-hydroxypyrrolidin-1-yl)-2,5-dioxo-2′,3′-dihydrospiro[imidazolidine-4,1′-indene]-1-yl)acetamide;
N-benzyl-N—((S)-1-cyclopropylethyl)-2-((S)-5′-(3-hydroxy-3-methylbut-1-ynyl)-2,5-dioxo-2′,3′-dihydrospiro[imidazolidine-4,1′-indene]-1-yl)acetamide;
N-benzyl-2-((S)-5′-cyano-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-((S)-5′-(3-methylureido)-2,5-dioxo-2′,3′-dihydrospiro[imidazolidine-4,1′-indene]-1-yl)acetamide;
N-benzyl-N—((S)-1-cyclopropylethyl)-2-(5′-(3-hydroxyazetidin-1-yl)-2,5-dioxo-2′,3′-dihydrospiro[imidazolidine-4,1′-indene]-1-yl)acetamide;
N-benzyl-2-((S)-5′-(2-cyanoacetamido)-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(5′-(2,4-dioxo-3,4-dihydropyrimidin-1(2H)-yl)-2,5-dioxo-2′,3′-dihydrospiro[imidazolidine-4,1′-indene]-1-yl)acetamide;
N-(1-(2-(benzyl((S)-1-cyclopropylethyl)amino)-2-oxoethyl)-2,5-dioxo-2′,3′-dihydrospiro[imidazolidine-4,1′-indene]-5′-yl)acrylamide;
N-benzyl-2-(5′-(2-cyano-1-hydroxyallyl)-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(5′-(2,4-dioxo-1,2,3,4-tetrahydropyrimidin-5-yl)-2,5-dioxo-2′,3′-dihydrospiro[imidazolidine-4,1′-indene]-1-yl)acetamide;
1-(2-(benzyl((S)-1-cyclopropylethyl)amino)-2-oxoethyl)-2,5-dioxo-2′,3′-dihydrospiro[imidazolidine-4,1′-indene]-5′-carboxylic acid;
(S)—N-(1-(2-(benzyl(1-cyclopropylethyl)amino)-2-oxoethyl)-2,5-dioxo-2′,3′-dihydrospiro[imidazolidine-4,1′-indene]-5′-yl)-3-oxobutanamide;
N-(1-(2-(benzyl((S)-1-cyclopropylethyl)amino)-2-oxoethyl)-2,5-dioxo-2′,3′-dihydrospiro[imidazolidine-4,1′-indene]-5′-yl)-2-cyano-3-oxobutanamide;
(E)-N-(1-(2-(benzyl((S)-1-cyclopropylethyl)amino)-2-oxoethyl)-2,5-dioxo-2′,3′-dihydrospiro[imidazolidine-4,1′-indene]-5′-yl)-4-(piperidin-1-yl)but-2-enamide;
2-amino-N-((1′S)-1-((4S)-3-benzyl-4-cyclopropyl-2-oxopentyl)-2,5-dioxo-2′,3′-dihydrospiro[imidazolidine-4,1′-indene]-5′-yl)-2-methylpropan amide;
(S)-2-amino-N—((S)-1-(2-(benzyl((S)-1-cyclopropylethyl)amino)-2-oxoethyl)-2,5-dioxo-2′,3′-dihydrospiro[imidazolidine-4,1′-indene]-5′-yl)propanamide;
N-benzyl-N—((S)-1-cyclopropylethyl)-2-(5′-(2-hydroxy-5-oxo-2,5-dihydrofuran-2-yl)-2,5-dioxo-2′,3′-dihydrospiro[imidazolidine-4,1′-indene]-1-yl)acetamide;
5′-(azetidin-3-ylmethylamino)-1-((4S)-3-benzyl-4-cyclopropyl-2-oxopentyl)-2′,3′-dihydrospiro[imidazolidine-4,1′-indene]-2,5-dione;
1-amino-N—((S)-1-(2-(benzyl((S)-1-cyclopropylethyl)amino)-2-oxoethyl)-2,5-dioxo-2′,3′-dihydrospiro[imidazolidine-4,1′-indene]-5′-yl)cyclobutanecarboxamide;
N-benzyl-N—((S)-1-cyclopropylethyl)-2-(2,5-dioxo-5′-(thiazol-2-ylamino)-2′,3′-dihydrospiro[imidazolidine-4,1′-indene]-1-yl)acetamide;
(S)-2-(5′-(azetidin-3-ylamino)-2,5-dioxo-2′,3′-dihydrospiro[imidazolidine-4,1′-indene]-1-yl)-N-benzyl-N-(1-cyclopropylethyl)acetamide;
N-benzyl-2-[5′-(5-cyano-6-oxo-1,6-dihydropyridin-3-yl)-2,5-dioxo-2′,3′-dihydrospiro[imidazolidine-4,1′-indene]-1-yl]-N-(1-cyclopropylethyl)acetamide;
(2R)-2-amino-N-[(4S)-1-({benzyl[(1S)-1-cyclopropylethyl]carbamoyl}methyl)-2,5-dioxo-2′,3′-dihydrospiro[imidazolidine-4,1′-indene]-5′-yl]propanamide;
N-benzyl-N-[(1S)-1-cyclopropylethyl]-2-{5′-[(1H-imidazol-2-yl)amino]-2,5-dioxo-2′,3′-dihydrospiro[imidazolidine-4,1′-indene]-1-yl}acetamide;
1-amino-N-[(4S)-1-({benzyl[(1S)-1-cyclopropylethyl]carbamoyl}methyl)-2,5-dioxo-2′,3′-dihydrospiro[imidazolidine-4,1′-indene]-5′-yl]cyclopropane-1-carboxamide;
N-benzyl-N-[(1S)-1-cyclopropylethyl]-2-{5′-[(oxetan-3-ylmethyl)amino]-2,5-dioxo-2′,3′-dihydrospiro[imidazolidine-4,1′-indene]-1-yl}acetamide;
2-[5′-(azetidin-3-yloxy)-2,5-dioxo-2′,3′-dihydrospiro[imidazolidine-4,1′-indene]-1-yl]-N-benzyl-N-[(1S)-1-cyclopropylethyl]acetamide;
N-benzyl-N-[(1R)-1-cyclopropyl-2,2,2-trifluoroethyl]-2-{1,1,2′,5′-tetraoxo-2H-spiro[1λ⁶-benzothiophene-3,4′-imidazolidine]-1′-yl}acetamide;
2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide;
N-[(1S)-1-cyclopropylethyl]-2-[2,5-dioxo-5′-(6-oxo-1,6-dihydropyridin-3-yl)-1′,3′-dihydro-1H-spiro[imidazolidine-4,2′-inden]-1-yl]-N-(4-fluorobenzyl)acetamide;
2-(5′-amino-2,5-dioxo-1′,3′-dihydro-1H-spiro[imidazolidine-4,2′-inden]-1-yl)-N-[(1S)-1-cyclopropylethyl]-N-(4-fluorobenzyl)acetamide;
N-[(1S)-1-cyclopropylethyl]-N-(4-fluorobenzyl)-2-{5′-[(methylcarbamoyl)amino]-2,5-dioxo-1′,3′-dihydro-1H-spiro[imidazolidine-4,2′-inden]-1-yl}acetamide;
2-[5′-(acetylamino)-2,5-dioxo-1′,3′-dihydro-1H-spiro[imidazolidine-4,2′-inden]-1-yl]-N-[(1S)-1-cyclopropylethyl]-N-(4-fluorobenzyl)acetamide;
2-[5′-(acetylamino)-2,5-dioxo-2′,3′-dihydro-1H-spiro[imidazolidine-4,1′-inden]-3-yl]-N-(4-fluorobenzyl)-N-[(2S)-1,1,1-trifluoropropan-2-yl]acetamide;
2-[5′-(acetylamino)-2,5-dioxo-2′,3′-dihydro-1H-spiro[imidazolidine-4,1′-indene]-1-yl]-N-[(1S)-1-cyclopropylethyl]-N-(4-fluorobenzyl)acetamide;
2-(6′-bromo-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
2-(6′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
N-[(1S)-1-cyclopropylethyl]-N-(4-fluorobenzyl)-2-{6′-[(methylcarbamoyl)amino]-2,5-dioxo-3′,4′-dihydro-1H,1′H-spiro[imidazolidine-4,2′-naphthalen]-1-yl}acetamide;
2-[6′-(acetylamino)-2,5-dioxo-3′,4′-dihydro-1H,1′H-spiro[imidazolidine-4,2′-naphthalen]-1-yl]-N-[(1S)-1-cyclopropylethyl]-N-(4-fluorobenzyl)acetamide;
2-[(4S)-5′-{1-[2-(dimethylamino)-2-oxoethyl]-1H-pyrazol-4-yl}-2,5-dioxo-2′,3′-dihydro-1H-spiro[imidazolidine-4,1′-inden]-3-yl]-N-(4-fluorobenzyl)-N-[(2S)-1,1,1-trifluoropropan-2-yl]acetamide;
2-(5′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-[(4S)-5′-bromo-2,5-dioxo-2′,3′-dihydro-1H-spiro[imidazolidine-4,1′-indene]-1-yl]-N-(4-fluorobenzyl)-N-[(2S)-1,1,1-trifluoropropan-2-yl]acetamide;
N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-{(4S)-5′-[1-(2-amino-2-oxoethyl)-1H-pyrazol-4-yl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(3,4-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(2,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-[(1S)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-[(1S)-1-cyclopropyl-2,2,2-trifluoroethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-[(1R)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-benzyl-N-[(1S)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-benzyl-N-[(1R)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide;
2-(5′-bromo-6′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-{2,5-dioxo-5′-[(2,2,2-trifluoroethyl)amino]-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-[(4S)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4R)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-(6′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
2-{5′-[1-(2-amino-2-oxoethyl)-1H-pyrazol-4-yl]-6′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-{6′-fluoro-5′-[1-(2-hydroxyethyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-[6′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-{3′,6′-difluoro-5′-[1-(2-hydroxyethyl)-1H-pyrazol-4-yl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-[(4R)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-[(4S)-2,5-dioxo-5′-(1H-pyrazol-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{5′-[(1-methyl-1H-pyrazol-3-yl)amino]-2,5-dioxo-2′,3′-dihydro-1H-spiro[imidazolidine-4,1′-inden]-1-yl}-N-[(2S)-1,1,1-trifluoropropan-2-yl]acetamide;
2-[(4S)-5′-amino-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
methyl trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylate;
trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylic acid;
trans-4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]cyclohexanecarboxylic acid;
trans-4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}cyclohexanecarboxylic acid;
ethyl ({4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]piperidin-1-yl}sulfonyl)carbamate;
2-[(4S)-5′-{[(2-cyanoethyl)carbamoyl]amino}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{(4S)-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
tert-butyl (4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetate;
(4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetic acid;
{4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
(4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}cyclohexylidene)acetic acid;
ethyl [(4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}piperidin-1-yl)sulfonyl]carbamate;
tert-butyl 4-[{[(4S)-5′-bromo-2,5-dioxo-2′,3′-dihydro-1H-spiro[imidazolidine-4,1′-inden]-1-yl]acetyl}(4-fluorobenzyl)amino]piperidine-1-carboxylate;
ethyl ({4-[{[(4S)-5′-amino-2,5-dioxo-2′,3′-dihydro-H-spiro[imidazolidine-4,1′-inden]-1-yl]acetyl}(4-fluorobenzyl)amino]piperidin-1-yl}sulfonyl)carbamate;
ethyl ({3,3-difluoro-4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]pyrrolidin-1-yl}sulfonyl)carbamate;
ethyl ({3,3-difluoro-4-[(4-fluorobenzyl) {[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]acetyl}amino]pyrrolidin-1-yl}sulfonyl)carbamate;
tert-butyl {4-[{[(4S)-5′-bromo-2,5-dioxo-2′,3′-dihydro-1H-spiro[imidazolidine-4,1′-inden]-1-yl]acetyl}(4-fluorobenzyl)amino]cyclohexylidene}acetate;
tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate;
{4-[{[(4S)-5′-amino-2,5-dioxo-2′,3′-dihydro-1H-spiro[imidazolidine-4,1′-inden]-1-yl]acetyl}(4-fluorobenzyl)amino]cyclohexylidene}acetic acid;
N-(4-fluorobenzyl)-2-{6′-fluoro-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
methyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate;
tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetate;
{4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetic acid;
2-[(4S)-2,5-dioxo-5′-(1H-pyrazol-5-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4S)-5′-{1-[2-(dimethylamino)ethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
tert-butyl 4-({4-[(4S)-1-(2-{(4-fluorobenzyl)[(2S)-1,1,1-trifluoropropan-2-yl]amino}-2-oxoethyl)-2,5-dioxo-2′,3′-dihydrospiro[imidazolidine-4,1′-inden]-5′-yl]-1H-pyrazol-1-yl}methyl)piperidine-1-carboxylate;
2-{(4S)-2,5-dioxo-5′-[1-(piperidin-4-ylmethyl)-1H-pyrazol-4-yl]-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-benzyl-N-(1-cyclopropylethyl)-2-[5′-(methylsulfonyl)-2,5-dioxo-2′,3′-dihydro-1H-spiro[imidazolidine-4,1′-inden]-1-yl]acetamide;
N-benzyl-N-(1-cyclopropylethyl)-2-[5′-(hydroxymethyl)-3-methyl-2,5-dioxo-2′,3′-dihydro-1H-spiro[imidazolidine-4,1′-inden]-1-yl]acetamide;
N-benzyl-N-(1-cyclopropylethyl)-2-[5′-(6-hydroxypyridin-3-yl)-2,5-dioxo-2′,3′-dihydro-1H-spiro[imidazolidine-4,1′-inden]-1-yl]acetamide;
N-benzyl-N-[(1S)-1-cyclopropylethyl]-2-[5′-(formylamino)-2,5-dioxo-2′,3′-dihydro-1H-spiro[imidazolidine-4,1′-inden]-1-yl]acetamide;
N-benzyl-2-[(4R)-5′-bromo-2,5-dioxo-2′,3′-dihydro-1H-spiro[imidazolidine-4,1′-inden]-1-yl]-N-[(1S)-1-cyclopropylethyl]acetamide;
2-[1,1-dioxido-2′,5′-dioxo-6-(pyridin-3-yl)-1H-spiro[1-benzothiophene-3,4′-imidazolidin]-1′-yl]-N-(4-fluorobenzyl)-N-[(2S)-1,1,1-trifluoropropan-2-yl]acetamide;
N-(4-fluorobenzyl)-2-[(3′S,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
(4-{[(5′-bromo-6′-fluoro-2,5-dioxo-2′,3′-dihydro-1H-spiro[imidazolidine-4,1′-inden]-1-yl)acetyl](4-fluorobenzyl)amino}cyclohexylidene)acetic acid;
{4-[(4-fluorobenzyl) {[6′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
tert-butyl (4-{[(5′-bromo-6′-fluoro-2,5-dioxo-2′,3′-dihydro-1H-spiro[imidazolidine-4,1′-inden]-1-yl)acetyl](4-fluorobenzyl)amino}cyclohexylidene)acetate; and pharmaceutically acceptable salts thereof.

In another aspect, the present invention relates to compounds of Formula (XIVa), Formula (XIVb), Formula (XVa), or Formula (XVb),

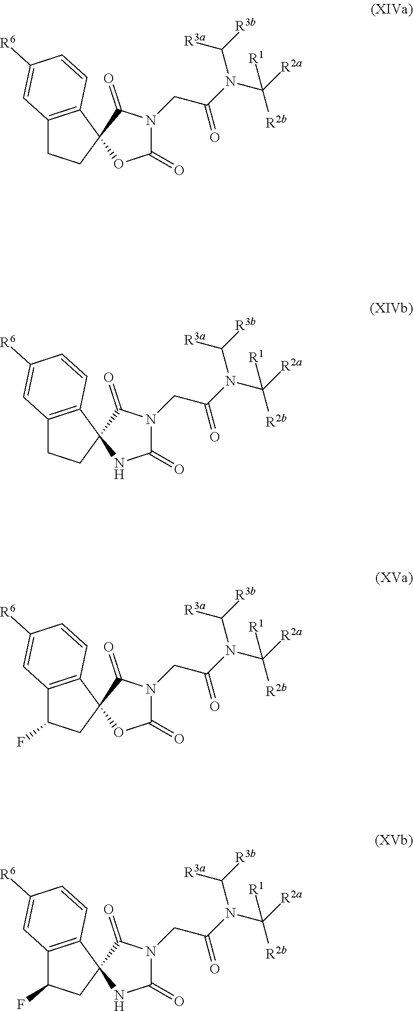

or a stereoisomer, tautomer or pharmaceutically acceptable salt thereof,
wherein
R¹ is carbocyclyl or heterocyclyl;
R^{2a} and R^{2b} are each independently H, D, or C₁-C₆ alkyl;
R^{3a} is hydrogen, C(O)NH₂, C₁-C₆ alkyl, aryl, cycloalkyl or heterocyclyl; and
R^{3b} is C₁-C₆ alkyl, aryl, cycloalkyl or heterocyclyl; or
R^{3a} and R^{3b} taken together with the carbon to which they are attached form an arene, cycloalkane, or heterocycle;
R⁶ is independently halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═S)N(R¹²)₂, —NHC(═N—C≡N)NR¹², —NHC(═N—C≡N)SR¹², or —NHS(O)ₘR¹²;
R¹¹ and R¹³ are each independently H, —OH, or C₁-C₆ alkyl; and
R¹², at each occurrence, is independently H, C₁-C₆ alkyl, aryl, cycloalkyl, or heterocyclyl.

with the proviso that R^{3a} and R^{3b} taken together with the carbon to which they are attached do not form tetrahydrothiophene 1,1-dioxide or tetrahydrothiophene when R¹ is unsubstituted phenyl.

In one embodiment of Formula (XIVa), (XIVb), (XVa) or (XVb), R¹ is carbocyclyl or heterocyclyl. In another embodiment of Formula (XIVa), (XIVb), (XVa) or (XVb), R¹ is carbocyclyl. In another embodiment of Formula (XIVa), (XIVb), (XVa) or (XVb), R¹ is heterocyclyl. In another embodiment of Formula (XIVa), (XIVb), (XVa) or (XVb), R¹ is phenyl, naphthyl, cyclopropyl, or cyclobutyl. In another embodiment of Formula (XIVa), (XIVb), (XVa) or (XVb), R¹ is phenyl. In another embodiment of Formula (XIVa), (XIVb), (XVa) or (XVb), R¹ is phenyl, which is unsubstituted. In another embodiment of Formula (XIVa), (XIVb), (XVa) or (XVb), R¹ is phenyl, which is substituted. In another embodiment of Formula (XIVa), (XIVb), (XVa) or (XVb), R¹ is phenyl, which is substituted with F, Cl, Br, CN, —NR_{g}Rₕ, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, aryl, and/or haloalkyl; wherein alkyl, alone or part of a group, is optionally substituted with F, C(═O)NR_{g}Rₕ, or CN. In another embodiment of Formula (XIVa), (XIVb), (XVa) or (XVb), R¹ is phenyl, which is substituted with F, Cl, Br, CN, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, and/or haloalkyl; wherein alkyl, alone or part of a group, is optionally substituted with F. In another embodiment of Formula (XIVa), (XIVb), (XVa) or (XVb), R¹ is phenyl, which is substituted with F. In another embodiment of Formula (XIVa), (XIVb), (XVa) or (XVb), R¹ is tetrahydropyranyl, thiazolyl, oxazolyl, thiadiazolyl, pyrimidinyl, thiophenyl, furanyl, triazolyl, indolyl, imidazolyl, or pyridinyl. In another embodiment of Formula (XIVa), (XIVb), (XVa) or (XVb), R¹ is tetrahydropyranyl, thiazolyl, oxazolyl, thiadiazolyl, pyrimidinyl, thiophenyl, furanyl, triazolyl, indolyl, imidazolyl, or pyridinyl; which is unsubstituted. In another embodiment of Formula (XIVa), (XIVb), (XVa) or (XVb), R¹ is tetrahydropyranyl, thiazolyl, oxazolyl, thiadiazolyl, pyrimidinyl, thiophenyl, furanyl, triazolyl, indolyl, imidazolyl, or pyridinyl; which is substituted. In another embodiment of Formula (XIVa), (XIVb), (XVa) or (XVb), R¹ is tetrahydropyranyl, thiazolyl, oxazolyl, thiadiazolyl, pyrimidinyl, thiophenyl, furanyl, triazolyl, indolyl, imidazolyl, or pyridinyl; which is substituted with F, Cl, Br, CN, —NR_{g}Rₕ, —NR_{g}C(═O)Rₕ, —NR_{g}C(═O)NR_{g}Rₕ, —OR_{g}, —SR_{g}, —SO₂R_{g}, —C(═O)OR_{g}, —C(═O)NR_{g}Rₕ, alkyl, or aryl; wherein R_{g} and Rₕ are the same or different and are independently hydrogen, alkyl, aryl, and/or haloalkyl; wherein alkyl, alone or part of a group, is optionally substituted with F, C(═O)NR_{g}Rₕ, or CN.

In one embodiment of Formula (XIVa), (XIVb), (XVa) or (XVb), R^{2a} and R^{2b} are each independently H, D, or C₁-C₆ alkyl. In another embodiment of Formula (XIVa), (XIVb), (XVa) or (XVb), R^{2a} and R^{2b} are each independently H. In another embodiment of Formula (XIVa), (XIVb), (XVa) or (XVb), R^{2a} is H and R^{2b} is C₁-C₆ alkyl. In another embodiment of Formula (XIVa), (XIVb), (XVa) or (XVb), R^{2a} is H and R^{2b} is C₁-alkyl.

In one embodiment of Formula (XIVa), (XIVb), (XVa) or (XVb), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; or R^{3a} and R^{3b} taken together with the carbon to which they are attached form arene, cycloalkane, or heterocycle. In another embodiment of Formula (XIVa), (XIVb), (XVa) or (XVb), R^{3a} is hydrogen, C(O)NH₂, C₁-C₆ alkyl, aryl, cycloalkyl or heterocyclyl; and R^{3b} is C₁-C₆ alkyl, aryl, cycloalkyl or heterocyclyl; wherein C₁-C₆ alkyl, is optionally substituted with F, —CN, —NR_{g}Rₕ, —OR_{g}, or —SO₂R_{g}; wherein R_{g} and Rₕ are the same or different and independently hydrogen or alkyl; wherein aryl, cycloalkyl and heterocyclyl are optionally substituted with alkyl, —SO₂NR_{g}C(═O)ORₕ, —SO₂NR_{g}Rₕ, —C(═O)R_{g}, —C(═O)OR_{g}, or —C(═O)NR_{g}Rₕ; wherein R_{g} and Rₕ are the same or different and independently hydrogen or alkyl; wherein alkyl, alone or part of a group, is optionally substituted with aryl or —C(═O)OH.

In another embodiment of Formula (XIVa), (XIVb), (XVa) or (XVb), R^{3a} is C₁-C₆ alkyl; and R^{3b} is C₁-C₆ alkyl; wherein the C₁-C₆ alkyl is optionally substituted with one or more F, —CN, alkoxy, or SO₂R_{g}. In another embodiment of Formula (XIVa), (XIVb), (XVa) or (XVb), R^{3a} is C₁-C₆ alkyl; and R^{3b} is CF₃. In another embodiment of Formula (XIVa), (XIVb), (XVa) or (XVb), R^{3a} is CH₃; and R^{3b} is CF₃. In another embodiment of Formula (XIVa), (XIVb), (XVa) or (XVb), R^{3a} is hydrogen; and R^{3b} is C₁-C₆ alkyl; wherein the C₁-C₆ alkyl is optionally substituted with one or more F, —CN, alkoxy, or SO₂R_{g}. In another embodiment of Formula (XIVa), (XIVb), (XVa) or (XVb), R^{3a} is hydrogen; and R^{3b} is cycloalkyl. In another embodiment of Formula (XIVa), (XIVb), (XVa) or (XVb), R^{3a} is hydrogen; and R^{3b} is cyclopropyl. In another embodiment of Formula (XIVa), (XIVb), (XVa) or (XVb), R^{3a} is C₁-C₆ alkyl; wherein the C₁-C₆ alkyl is CH₃; and R^{3b} is cycloalkyl. In another embodiment of Formula (XIVa), (XIVb), (XVa) or (XVb), R^{3a} is CH₃; and R^{3b} is cyclopropyl. In another embodiment of Formula (XIVa), (XIVb), (XVa) or (XVb), R^{3a} is CF₃; and R^{3b} is cyclopropyl. In another embodiment of Formula (XIVa), (XIVb), (XVa) or (XVb), R^{3a} is cycloalkyl; and R^{3b} is cycloalkyl. In another embodiment of Formula (XIVa), (XIVb), (XVa) or (XVb), R^{3a} is cyclopropyl; and R^{3b} is cyclopropyl. In another embodiment of Formula (XIVa), (XIVb), (XVa) or (XVb), R^{3a} is C₁-C₆ alkyl; and R^{3b} is heterocyclyl. In another embodiment of Formula (XIVa), (XIVb), (XVa) or (XVb), R^{3a} is hydrogen; and R^{3b} is aryl. In another embodiment of Formula (XIVa), (XIVb), (XVa) or (XVb), R^{3a} and R^{3b} taken together with the carbon to which they are attached form arene, cycloalkane, or heterocycle. In another embodiment of Formula (XIVa), (XIVb), (XVa) or (XVb), R^{3a} and R^{3b} taken together with the carbon to which they are attached form arene. In another embodiment of Formula (XIVa), (XIVb), (XVa) or (XVb), R^{3a} and R^{3b} taken together with the carbon to which they are attached form cycloalkane. In another embodiment of Formula (XIVa), (XIVb), (XVa) or (XVb), R^{3a} and R^{3b} taken together with the carbon to which they are attached form heterocycle. In another embodiment of Formula (XIVa), (XIVb), (XVa) or (XVb), R^{3a} and R^{3b} taken together with the carbon to which they are attached form cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, or benzene. In another embodiment of Formula (XIVa), (XIVb), (XVa) or (XVb), R^{3a} and R^{3b} taken together with the carbon to which they are attached form cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, or benzene wherein cyclopropane, azetidine, cyclobutane, tetrahydrofuran, pyrrolidine, cyclopentane, cyclohexane, 2-azaspiro[3.3]heptane, tetrahydro-2H-thiopyran 1,1 dioxide, piperidine, and benzene are optionally substituted with alkyl, haloalkyl, F, CN, —NR_{g}Rₕ, —NR_{g}C(═O)Rₕ, —NR_{g}SO₂Rₕ, —OR_{g}, —SO₂R_{g}, —NR_{g}SO₂NR_{g}C(═O)Rₕ, —SO₂NR_{g}C(═O)ORₕ, —SO₂NR_{g}Rₕ, —C(═O)R_{g}, —C(═O)OR_{g}, or —C(═O)NR_{g}Rₕ; wherein R_{g} and Rₕ are the same or different and independently hydrogen, alkyl, or haloalkyl; wherein each alkyl, alone or part of a group, is optionally substituted with CN, alkyl, —SO₂NH₂, C(═O)NHCH₃, —C(═O)OC(CH₃)₃, —C(═O)OCH₃, or —C(═O)OH.

In one embodiment of Formula (XIVa), (XIVb), (XVa) or (XVb), R⁶ is independently halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R²)₂, —NHC(═S)N(R¹²)₂, —NHC(═N—C≡N)NR¹², —NHC(═N—C≡N)SR¹², or —NHS(O)ₘR¹². In another embodiment of Formula (XIVa), (XIVb), (XVa) or (XVb), R⁶ is halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —NHC(═S)N(R²)₂, —NHC(═N—C≡N)NR², —NHC(═N—C≡N)SR², or —NHS(O)ₘR². In another embodiment of Formula (XIVa), (XIVb), (XVa) or (XVb), R⁶ is —N(R¹²)₂, —C(═O)NHR¹², —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R²)₂, —NHC(═O)N(R¹²)₂, —NHC(═S)N(R¹²)₂, —NHC(═N—C≡N)NR¹², —NHC(═N—C≡N)SR¹², or —NHS(O)ₘR¹². In another embodiment of Formula (XIVa), (XIVb), (XVa) or (XVb), R⁶ is heterocyclyl. In another embodiment of Formula (XIVa), (XIVb), (XVa) or (XVb), R⁶ is pyrazolyl. In another embodiment of Formula (XIVa), (XIVb), (XVa) or (XVb), R⁶ is —NHC(═O)NHR¹².

In one embodiment of Formula (XIVa), (XIVb), (XVa) or (XVb), R¹¹ and R¹³ are each independently H, —OH, or C₁-C₆ alkyl. In another embodiment of Formula (XIVa), (XIVb), (XVa) or (XVb), R¹¹ and R¹³ are each independently H. In another embodiment of Formula (XIVa), (XIVb), (XVa) or (XVb), R¹¹ and R¹³ are each independently —OH. In another embodiment of Formula (XIVa), (XIVb), (XVa) or (XVb), R¹¹ and R¹³ are each independently C₁-C₆ alkyl.

In one embodiment of Formula (XIVa), (XIVb), (XVa) or (XVb), R¹², at each occurrence, is independently H, C₁-C₆ alkyl, aryl, cycloalkyl, or heterocyclyl. In another embodiment of Formula (XIVa), (XIVb), (XVa) or (XVb), R¹², at each occurrence, is independently H, C₁-C₆ alkyl, aryl, cycloalkyl, or heterocyclyl; wherein C₁-C₆ alkyl, aryl, cycloalkyl, and heterocyclyl are optionally substituted with F, alkyl, alkoxy, cycloalkyl, haloalkyl, heterocyclyl, heterocyclylalkyl, oxo, CN, —NR_{g}Rₕ, —NR_{g}C(═O)Rₕ, —NR_{g}C(═O)ORₕ, —OR_{g}, —SO₂R_{g}, —SO₂NR_{g}Rₕ, —C(═O)R_{g}, —C(═O)OR_{g}, or —C(═O)NR_{g}Rₕ; wherein R_{g} and Rₕ are the same or different and independently hydrogen, alkyl, alkoxy, aryl, cycloalkyl, and/or haloalkyl.

Still another embodiment pertains to compounds of Formula (XIVa), selected from the group consisting of:
2-((S)-5-acetamido-2′,4′-dioxo-2,3-dihydrospiro[indene-1,5′-ozazolidine]-3′-yl)-N-benzyl-N—((S)-1-cyclopropylethyl)acetamide;
N-benzyl-N-[(1S)-1-cyclopropylethyl]-2-[(1S)-5-[(methylcarbamoyl)amino]-3′,5′-dioxo-2,3-dihydrospiro[indene-1,2′-[1,4]ozazolidine]-4′-yl]acetamide;
2-amino-N-[(1R)-4′-({[(4-bromophenyl)methyl][(1S)-1-cyclopropylethyl]carbamoyl}methyl)-3′,5′-dioxo-2,3-dihydrospiro[indene-1,2′-[1,4]ozazolidine]-5-yl]-2′-methylpropanamide;
N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]-N-[(2S)-1,1,1-trifluoropropan-2-yl]acetamide;
N-[(1S)-1-cyclopropylethyl]-N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]acetamide;
(2S)-2-amino-N-[(1S)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1S)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
N-benzyl-N-[(1R)-1-cyclopropyl-2,2,2-trifluoroethyl]-2-[(1S)-5-[(methylcarbamoyl)amino]-3′,5′-dioxo-2,3-dihydrospiro[indene-1,2′-[1,4]ozazolidine]-4′-yl]acetamide;
(2R)-2-amino-N-[(1S)-4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1S)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(1R)-1-cyclopropyl-2,2,2-trifluoroethyl]-N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]acetamide;
N-[(1S)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
N-[(1 S)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1R)-3′-(2-{[(1R)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-benzyl-2-[(1R)-5-bromo-2′,4′-dioxo-2,3-dihydro-3′H-spiro[indene-1,5′-[1,3]ozazolidin]-3′-yl]-N-[(1S)-1-cyclopropylethyl]acetamide;
2-[(1R)-5-amino-2′,4′-dioxo-2,3-dihydro-3′H-spiro[indene-1,5′-[1,3]ozazolidin]-3′-yl]-N-[(1R)-1-cyclopropyl ethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-[(1R)-1-cyclopropylethyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1R)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]azetidine-1-carboxylate;
2-[(1R)-5-(6-aminopyridin-3-yl)-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-{(1R)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-[(1S)-1-cyclopropylethyl]-N-(4-fluorobenzyl)acetamide;
N-[(1S)-1-cyclopropylethyl]-2-[(1R)-2′,4′-dioxo-5-(pyridin-3-yl)-2,3-dihydro-3′H-spiro[indene-1,5′]-[1,3]ozazolidin]-3′-yl]-N-(4-fluorobenzyl)acetamide;
2-[(1R)-5-(azetidin-3-yl)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
2-[(1R)-5-(1-acetylazetidin-3-yl)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-benzyl-N-[(1S)-1-cyclopropylethyl]-2-[(1R)-5-(1-methylazetidin-3-yl)-2′,4′-dioxo-2,3-dihydro-3′H-spiro[indene-1,5′-[1,3]ozazolidin]-3′-yl]acetamide;
3-[(1R)-3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]-N-methylazetidine-1-carboxamide;
N-benzyl-2-{(1R)-5-[1-(cyclopropylcarbonyl)azetidin-3-yl]-2′,4′-dioxo-2,3-dihydro-3′H-spiro[indene-1,5′-[1,3]ozazolidin]-3′-yl}-N-[(1S)-1-cyclopropylethyl]acetamide;
N-benzyl-N-[(1S)-1-cyclopropylethyl]-2-{(1R)-5-[1-(2-hydroxy-2-methylpropanoyl)azetidin-3-yl]-2′,4′-dioxo-2,3-dihydro-3′H-spiro[indene-1,5′-[1,3]ozazolidin]-3′-yl}acetamide;
N-benzyl-N-[(1S)-1-cyclopropylethyl]-2-{(1R)-5-[1-(methylsulfonyl)azetidin-3-yl]-2′,4′-dioxo-2,3-dihydro-3′H-spiro[indene-1,5′-[1,3]ozazolidin]-3′-yl}acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]cyclobutanecarboxamide;
3-ethoxy-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-5-oxo-D-prolinamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-5-oxo-L-prolinamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]cyclopropanecarboxamide;
N-(4-fluorobenzyl)-2-{(1R)-5-[(methoxyacetyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(1R)-5-[(ethoxyacetyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-3-methylbutanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylpropanamide;
2-[(1R)-5-(acetylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-{[(2-methoxyethyl)(methyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[(dimethylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-{[methyl(2-methylpropyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(cyclopropylmethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(cyclobutylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-({[(2R)-1-hydroxy-3-methylbutan-2-yl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-({[(2S)-tetrahydrofuran-2-ylmethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-({[(2R)-tetrahydrofuran-2-ylmethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-({[2-(propan-2-yloxy)ethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(cyclopropylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-{[ethyl(methyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(diethylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyrrolidine-1-carboxamide;
2-[(1R)-5-({[2-(dimethylamino)ethyl](methyl)carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N²-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-L-leucinamide;
N-(4-fluorobenzyl)-2-[(1R)-5-{[(2-hydroxyethyl)(propyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(2-methoxyethyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(3-hydroxypropyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-({[(2S)-1-hydroxy-3-methylbutan-2-yl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[methyl(propan-2-yl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(2-cyanoethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-{[methyl(propyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-[(1S)-1-cyclopropylethyl]-2-[(1R)-2′,4′-dioxo-5-(pyridin-4-yl)-2,3-dihydro-3′H-spiro[indene-1,5′-[1,3]ozazolidin]-3′-yl]-N-(4-fluorobenzyl)acetamide;
N-[(1S)-1-cyclopropylethyl]-N-(4-fluorobenzyl)-2-[(1R)-5-(6-hydroxypyridazin-3-yl)-2′,4′-dioxo-2,3-dihydro-3′H-spiro[indene-1,5′-[1,3]ozazolidin]-3′-yl]acetamide;
N-[(1S)-1-cyclopropylethyl]-2-[(1R)-2′,4′-dioxo-5-(1H-pyrazol-4-yl)-2,3-dihydro-3′H-spiro[indene-1,5′-[1,3]ozazolidin]-3′-yl]-N-(4-fluorobenzyl)acetamide;
2-{(1R)-2′,4′-dioxo-5-[(tetrahydrofuran-2-ylmethyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[(cyclopentylmethyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[(2,2-dimethylbutyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-2′,4′-dioxo-5-[(tetrahydrofuran-3-ylmethyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-{(1R)-5-[(2-methylpropyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-[(1S)-1-cyclopropylethyl]-2-[(1R)-2′,4′-dioxo-5-(pyridin-2-yl)-2,3-dihydro-3′H-spiro[indene-1,5′-[1,3]ozazolidin]-3′-yl]-N-(4-fluorobenzyl)acetamide;
N-[(1S)-1-cyclopropylethyl]-N-(3-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(1R)-5-(carbamoylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-((R)-5-(3-(N-methylsulfamoyl)ureido)-2′,4′-dioxo-2,3-dihydrospiro[indene-1,5′-ozazolidine]-3′-yl)-N—((S)-1,1,1-trifluoropropan-2-yl)acetamide
methyl {[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}sulfamate;
2-((R)-5-(3-(N-(cyclopropylmethyl)sulfamoyl)ureido)-2′,4′-dioxo-2,3-dihydrospiro[indene-1,5′-ozazolidine]-3′-yl)-N-(4-fluorobenzyl)-N—((S)-1,1,1-trifluoropropan-2-yl)acetamide
(1R)-3′-(2-{benzyl[(1S)-1-cyclopropylethyl]amino}-2-oxoethyl)-N-(2-methylpropyl)-2′,4′-dioxo-2,3-dihydrospiro[indene-1,5′-[1,3]ozazolidine]-5-carboxamide;
N-[(1S)-1-cyclopropylethyl]-N-(2-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,4-difluorobenzyl)-2-{(1R)-5-[(methylcarbonyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,3-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-chloro-4-fluorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3-chloro-4-fluorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-[4-(trifluoromethyl)benzyl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1-methyl-1H-pyrazole-4-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1-methyl-1H-pyrazole-3-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-4-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-2-carboxamide;
(1R)-3′-(2-{benzyl[(1S)-1-cyclopropylethyl]amino}-2-oxoethyl)-N-[(2R)-1-hydroxy-3-methylbutan-2-yl]-2′,4′-dioxo-2,3-dihydrospiro[indene-1,5′-[1,3]ozazolidine]-5-carboxamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-[(5-methyl-1,3,4-thiadiazol-2-yl)methyl]acetamide;
N-(4-cyanobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3-methylbenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3,5-difluorobenzyl)-2-{(1R)-5-[(methylcarbamoyl)amino]-2′,4′-dioxo-2,4-dihydro-3′H-spiro[indene-1,5′-[1,3]ozazolidin]-3′-yl}acetamide;
N-(3-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,4-dichlorobenzyl)-2-{(1R)-5-[(methylcarbamoyl)amino]2′,4′-dioxo-2,3-dihydro-3′H-spiro[indene-1,5′-[1,3]ozazolidin]-3′-yl}acetamide;
N-[(1S)-1-cyclopropylethyl]-N-(4-methoxybenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3-methoxybenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-methylbenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-[(1R)-5-{[(1-methyl-1H-pyrazol-4-yl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-2′,4′-dioxo-5-[(phenylcarbamoyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-2′,4′-dioxo-5-[(pyridin-3-ylcarbamoyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-[(1S)-1-cyclopropylethyl]-N-(4-fluorobenzyl)-2-[(1R)-5-{1-[2-(methylamino)-2-oxoethyl]-1H-pyrazol-4-yl]}-2′,4′-dioxo-2,3-dihydro-3′H-spiro[indene-1,5′-[1,3]ozazolidin]-3′-yl]acetamide;
N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(dimethylamino)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(4-methylpyrimidin-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[5-(difluoromethyl)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(6-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1R)-5-[1-(2-methylpropy 1)-1H-pyrazol-4-yl]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(1R)-5-[4-(difluoromethyl)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(3-fluoropyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(1,3-thiazol-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-N-methylbenzamide;
2-{(1R)-5-[2-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(5-cyanopyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(1-methyl-1H-1,2,4-triazol-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(5-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-(6-cyanopyridin-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(5-fluoro-3-methylpyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(5-methyl-1,3,4-thiadiazol-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(4-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(6-methoxypyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[2-(dimethylamino)pyrimidin-5-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(acetylamino)pyridin-3-yl]-2′,4′-dioxo-2,3-dihydro-3′H-spiro[indene-1,5-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(1-methyl-1H-1,2,4-triazol-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-cyclopropyl-5-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]furan-2-carboxamide;
N-(4-fluorobenzyl)-2-[(1R)-5-(5-fluoropyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
4-fluoro-3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
5-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-N-methylpyridine-2-carboxamide;
6-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-3-carboxamide;
N-(4-fluorobenzyl)-2-[(1R)-5-(2-methoxypyrimidin-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
6-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-2-carboxamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]thiophene-3-carboxamide;
2-{(3R)-5-[4-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(1,3-dimethyl-2,4-dioxo-1,2,3,4-tetrahydropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4-cyanopyridin-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-4-carboxamide;
2-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
2-{(1R)-5-[3-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(6-cyano-5-methoxypyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(1-methyl-1H-pyrazol-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(1,3,5-trimethyl-1H-pyrazol-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(3,4-difluorophenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2,5-difluoro-4-methoxyphenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyrimidin-5-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-2′,4′-dioxo-5-[5-(trifluoromethyl)pyridin-3-yl]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-2′,4′-dioxo-5-(1,3-thiazol-2-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2-cyanopyridin-4-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyrazin-2-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[4-(carbamoylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(2-cyanopyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(6-cyano-5-fluoropyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
2-[(1R)-5-{[(4-aminophenyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(pyrimidin-5-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(pyrimidin-2-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-thiazol-5-ylmethyl)acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]prolinamide;
N²-(4-fluorobenzyl)-N²-({(1R)-5-[(methylcarbamoyl)amino]-2′,4′-dioxo-2,3-dihydro-3′H-spiro[indene-1,5′-[1,3]ozazolidin]-3′-yl}acetyl)-L-alaninamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1H-pyrazole-5-carboxamide;
N-(4-fluorobenzyl)-2-[(1R)-5-{[(methylsulfamoyl)acetyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(methylsulfonyl)acetyl]amino}-2′,4′-dioxo-2,3-dihydro-3′H-spiro[indene-1,5′-[1,3]ozazolidin]-3′-yl]-N-[(2S)-1,1,1-trifluoropropan-2′-yl]acetamide;
2-[(1R)-5-{[(dimethylsulfamoyl)acetyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-2′,4′-dioxo-5-[(sulfamoylacetyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidine-1-carboxamide
N-benzyl-N-ethyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
methyl N′-cyano-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamimidothioate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanamide;
N-(4-fluorobenzyl)-2-{(1R)-5-[(methylcarbamothioyl)amino]-2′,4′-dioxo-2,3-dihydro-3′H-spiro[indene-1,3′-[1,3]ozazolidin]-3′-yl}-N-[(2S)-1,1,1-trifluoropropan-2-yl]acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(tetrahydro-2H-pyran-4-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-oxazol-4-ylmethyl)acetamide;
2-[(1R)-5-(5-cyanothiophen-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4-cyano-3-fluorophenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(5-cyanothiophen-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(N″-cyano-N′-methylcarbamimidamido)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4,6-difluoropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[2-(acetylamino)-5-methylpyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(5-fluoropyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(furan-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(2-oxo-2,3-dihydro-1H-benzimidazol-5-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(4-methylpyrimidin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-2′,4′-dioxo-5-[1-(tetrahydro-2H-pyridin-2-yl)-1H-pyrazol-3-yl]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(5-fluoro-6-methylpyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[6-(difluoromethyl)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(difluoromethyl)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(furan-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[1-(ethoxymethyl)-1H-imidazol-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(5-cyano-1,2-dimethyl-6-oxo-1,6-dihydropyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]thiophene-2-carboxamide;
2-[(1R)-5-(2,6-dioxo-1,2,5,6-tetrahydropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2,4-dimethyl-1,3-thiazol-5-yl)-2′,4′-dioxo-2,3-dihydro-3′H-spiro[indene-1,5′-[1,3]ozazolidin]-3′-yl]-N-(4-fluorobenzyl)-N-[(2R)-1,1,1-trifluoropropan-2-yl]acetamide;
2-{(1R)-5-[1-(cyanomethyl)-3,5-dimethyl-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[3-(carbamoylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-[3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)-2,2-dimethylpropyl]propanamide;
(1R)-3′-(2-{benzyl[(1S)-1-cyclopropylethyl]amino}-2-oxoethyl)-N-methyl-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(propan-2-yl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(2-methoxyethyl)-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(tetrahydrofuran-2-ylmethyl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(tetrahydrofuran-3-ylmethyl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(cyanomethyl)-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(3-hydroxypropyl)-2′,4′-dioxo-2,3-dihydrospiro[indene-1,5′-[1,3]ozazolidine]-5-carboxamide;
3′-{2-[benzyl(1-cyclopropylethyl)amino]-2-oxoethyl}-N-(1-hydroxy-3-methylbutan-2-yl)-2′,4′-dioxo-2,3-dihydrospiro[indene-1,5′-[1,3]ozazolidine]-5-carboxamide;
N-(1-amino-4-methyl-1-oxopentan-2-yl)-3′-{2-[benzyl(1-cyclopropylethyl)amino]-2-oxoethyl}-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-[2-(diethylamino)ethyl]-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-[3-(1H-imidazol-1-yl)propyl]-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-[3-(2-oxopyrrolidin-1-yl)propyl]-2,3-dihydrospiro[indene-1,5′-[1,3]ozazolidine]-5-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]tetrahydropyrimidine-1(2H)-carboxamide;
3-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]tetrahydropyrimidine-1(2H)-carboxamide;
N-(4-fluorobenzyl)-2-{(1R)-5-[1-(2-hydroxyethyl)-1H-pyrazol-4-yl]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-phenylacetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N,N-dibenzyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
(2R)-3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-hydroxy-2-methylpropanamide;
(2S)-3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-hydroxy-2-methylpropanamide;
2-[(1R)-2′,4′-dioxo-5-({[3-(pyrrolidin-1-ylmethyl)phenyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-({[3-(morpholin-4-ylmethyl)phenyl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N³-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-N-methyl-beta-alaninamide;
N-[3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)phenyl]propanamide;
N-(2-chlorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)acetamide;
2-[(1R)-5-(4,5-dihydro-1H-imidazol-2-ylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-({[3-(acetylamino)propyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-({[2-(acetylamino)ethyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
tert-butyl [3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)-2,2-dimethylpropyl]carbamate;
N-[2-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)ethyl]propanamide;
2-[(1R)-5-{[(3-amino-2,2-dimethylpropyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-({[3-(acetylamino)phenyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
methyl 2-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]amino}-4,5-dihydro-1H-imidazole-1-carboxylate;
N-cyclohexyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-cyclopentyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-[1-(furan-2-yl)ethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-beta-alanine;
N-cyclopropyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl N-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-beta-alaninate;
N-(3,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2′-yl]acetamide;
N-(4-fluorobenzyl)-N-(3-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(1R)-5-amino-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
ethyl ({(3R)-3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(3R)-piperidin-3-yl]acetamide;
ethyl ({(3R)-3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]piperidin-1-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(methylsulfonyl)piperidin-4-yl]acetamide;
N-(cyclopropylmethyl)-2{(1R)-5-[(methylcarbamoyl)amino]-2′,4′-dioxo-2,3-dihydro-3′H-spiro[indene-1,5′-[1,3]ozazolidin]-3′-yl}-N-(1-phenylethyl)acetamide;
2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)-N-[2-(trifluoromethyl)benzyl]acetamide;
N-benzyl-N-(cyanomethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-(2-cyanoethyl)-2-{(1R)-5-[(methylcarbamoyl)amino]-2′,4′-dioxo-2,3-dihydro-3′H-spiro[indene-1,5″-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(methylsulfonyl)propan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(1-methyl-1H-pyrazol-4-yl)methyl]acetamide;
2-[(1R)-5-({[3-(acetylamino)-2,2-dimethylpropyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclopentyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(pyridin-3-yl)acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1-methyl-1H-pyrazol-4-yl)acetamide;
methyl [(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamate;
4-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)butanoic acid;
N²-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylalaninamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylalaninamide;
N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
N-cyclopentyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-oxazol-4-ylmethyl)acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-[1-(pyridin-2-yl)ethyl]acetamide;
2-[(1R)-5-{[(cyanomethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclohexyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-pyrrolidin-3-yl]acetamide;
N-(4-fluorobenzyl)-N-(trans-3-hydroxycyclobutyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-1-(methylsulfonyl)pyrrolidin-3-yl]acetamide;
ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidin-1-yl}sulfonyl)carbamate;
N-(1-cyanopropan-2-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
5-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)pentanoic acid;
N-[(3R)-1-acetylpyrrolidin-3-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-1-(2-sulfamoylethyl)pyrrolidin-3-yl]acetamide;
N-(4-fluorobenzyl)-N-(trans-4-hydroxy cyclohexyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
methyl (1R,3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-3-(methylsulfonyl)tetrahydropyrimidine-1(2H)-carboxamide;
N²-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]glycinamide;
2-{(1R)-5-[(methylcarbamoyl)amino]-2′,4′-dioxo-2,3-dihydro-3′H-spiro[indene-1,5′-[1,3]ozazolidin]-3′-yl}-N-[1-(2-methylphenyl)ethyl]-N-(2-methylpropyl)acetamide;
N-(2-chlorobenzyl)-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)acetamide;
N-(4-fluorobenzyl)-N-(4-methoxyphenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(4-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(2-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(trifluoromethyl)cyclopropyl]acetamide;
2-[(1R)-5-(3,4-dihydro-2H-pyrrol-5-ylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(3R)-pyrrolidin-3-yl]acetamide;
ethyl ({(3R)-3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]pyrrolidin-1-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(piperidin-4-yl)acetamide;
ethyl ({4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]azetidine-1-carboxylate;
N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}-N-[2-(trifluoromethyl)benzyl]acetamide;
N-(2-chloro-4-fluorobenzyl)-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(3-methoxyphenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-(pyridin-4-yl)acetamide;
methyl (1S,3S)-3-[(4-fluorobenzyl)({(1R)-5-[(methylcarbamoyl)amino]2′,4′-dioxo-2,3-dihydro-3′H-spiro[indene-1,5′-[1,3]ozazolidin]-3′-yl}acetyl)amino]cyclohexanecarboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(tetrahydro-2H-pyran-4-yl)acetamide;
N-(1,1-dioxidotetrahydro-2H-thiopyran-4-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2,2-difluorocyclopentyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-cyclohexyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-(4-fluorobenzyl)-N-{1-[2-(methylamino)-2-oxoethyl]-1H-pyrazol-4-yl}-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[1-(cyanomethyl)-1H-pyrazol-4-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl 3-{1-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]ethyl}azetidine-1-carboxylate;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylic acid;
ethyl ({(3S)-3-[(4-fluorobenzyl) {[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
ethyl ({(3S)-3-[{[(1R)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}(4-fluorobenzyl)amino]pyrrolidin-1-yl}sulfonyl)carbamate;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}acetate;
ethyl ({(3R)-3-[(4-fluorobenzyl) {[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]piperidin-1-yl}sulfonyl)carbamate;
ethyl ({(3R)-3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]piperidin-1-yl}sulfonyl)carbamate;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}acetic acid;
tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
tert-butyl (3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-piperidin-3-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3S)-piperidin-3-yl]acetamide;
N-[4-amino-3-(hydroxymethyl)butan-2-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({(3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzamide;
tert-butyl 2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(pyrrolidin-2-ylmethyl)acetamide;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexylidene}acetate;
N-ethyl-4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxamide;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexylidene}acetic acid;
N-(4-fluorobenzyl)-2-[(1R)-5-(oxetan-3-ylamino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzamide;
N-[(1-acetylpyrrolidin-2-yl)methyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{[1-(methylsulfonyl)pyrrolidin-2-yl]methyl}acetamide;
2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}-N-methylpyrrolidine-1-carboxamide;
N-(3,4-difluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{[1-(methylsulfamoyl)pyrrolidin-2-yl]methyl}acetamide;
N-(cyclopropylmethyl)-N-(2,3-dihydro-1H-inden-1-yl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexyl}acetic acid;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-3-methylazetidin-1-yl}sulfonyl)carbamate;
N-(3-cyanophenyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
tert-butyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}carbamate;
N-(3-aminocyclobutyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[3-(acetylamino)cyclobutyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{3-[(methylsulfonyl)amino]cyclobutyl}acetamide;
2,2′-({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}imino)diacetic acid;
N-{3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}glycine;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxylate;
methyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}carbamate;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}sulfamoyl)carbamate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxylic acid;
ethyl ({4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]piperidin-1-yl}sulfonyl)carbamate;
benzyl 3-{[(4-fluorobenzyl)({(1R)-5-[(methylcarbamoyl)amino]-2′,4′-dioxo-2,3-dihydro-3′H-spiro[indene-,5′-[1,3]ozazolidin]-3′-yl}acetyl)amino]methyl}azetidine-1-carboxylate;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoate;
ethyl ({3,3-difluoro-4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
ethyl ({3,3-difluoro-4-[(4-fluorobenzyl){[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
N-(4-fluorobenzyl)-N-(3-methoxycyclobutyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-N-methylcyclobutanecarboxamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxamide;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetic acid;
(2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidin-1-yl)acetic acid;
methyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetate;
{3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetic acid;
tert-butyl {4-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]cyclohexylidene}acetate;
tert-butyl {4-[(4-fluorobenzyl) {[(1S)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetate;
{4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetic acid;
N-(azetidin-3-ylmethyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl {trans-4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexyl}carbamate;
N-(trans-4-aminocyclohexyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{trans-4-[(methylsulfonyl)amino]cyclohexyl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(trans-4-{[(trifluoromethyl)sulfonyl]amino}cyclohexyl)acetamide;
tert-butyl 6-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-2-azaspiro[3.3]heptane-2-carboxylate;
N-(2-azaspiro[3.3]hept-6-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1-acetylazetidin-3-yl)methyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl [(3-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}azetidin-1-yl)sulfonyl]carbamate;
ethyl [(2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidin-1-yl)sulfonyl]carbamate;
ethyl ({6-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-2-azaspiro[3.3]hept-2-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(3-{[(trifluoromethyl)sulfonyl]amino}cyclobutyl)acetamide;
tert-butyl 3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]benzoate;
tert-butyl 3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]benzoate;
3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]benzoic acid;
N-(azetidin-3-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]azetidin-1-yl}sulfonyl)carbamate;
2-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
methyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanoate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanoic acid;
2-[(1R)-2′,4′-dioxo-5-(pyridin-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(6-oxo-1,6-dihydropyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-phenyl-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclohexyl-N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-(tetrahydro-2H-pyran-4-yl)acetamide;
3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclobutanecarboxylic acid;
4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexanecarboxylic acid;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-{trans-4-[(methylsulfonyl)amino]cyclohexyl}acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-(3-{[(trifluoromethyl)sulfonyl]amino}cyclobutyl)acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-[1-(methylsulfonyl)piperidin-4-yl]acetamide;
2,2′-({3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclobutyl}imino)diacetic acid;
N-(4-fluorobenzyl)-2-[(1R)-5-(1,2-oxazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide; and pharmaceutically acceptable salts thereof.

Still another embodiment pertains to compounds of Formula (XIVb), selected from the group consisting of:
N-benzyl-N—((S)-1-cyclopropylethyl)-2-((S)-2,5-dioxo-5′-(6-oxo-1,6-dihydropyridin-3-yl)-2′,3′-dihydrospiro[imidazolidine-4,1′-indene]-1-yl)acetamide;
N-benzyl-N—((S)-1-cyclopropylethyl)-2-((S)-5′-(3-hydroxyprop-1-ynyl)-2,5-dioxo-2′,3′-dihydrospiro[imidazolidine-4,1′-indene]-1-yl)acetamide;
N-benzyl-N—((S)-1-cyclopropylethyl)-2-((S)-2,5-dioxo-5′-(2H-1,2,3-triazol-4-yl)-2′,3′-dihydrospiro[imidazolidine-4,1′-indene]-1-yl)acetamide;
N-benzyl-N—((S)-1-cyclopropylethyl)-2-((S)-5′-(3-hydroxy-3-methylbut-1-ynyl)-2,5-dioxo-2′,3′-dihydrospiro[imidazolidine-4,1′-indene]-1-yl)acetamide;
N-benzyl-2-((S)-5′-cyano-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-((S)-5′-(3-methylureido)-2,5-dioxo-2′,3′-dihydrospiro[imidazolidine-4,1′-indene]-1-yl)acetamide;
N-benzyl-2-((S)-5′-(2-cyanoacetamido)-2,5-dioxo-2′,3′-dihydrospiro[imidazolidine-4,1′-indene]-1-yl)-N—((S)-1-cyclopropylethyl)acetamide;
2-amino-N-((1′S)-1-((4S)-3-benzyl-4-cyclopropyl-2-oxopentyl)-2,5-dioxo-2′,3′-dihydrospiro[imidazolidine-4, 1′-indene]-5′-yl)-2-methylpropanamide;
(2R)-2-amino-N-[(4 S)-1-({benzyl[(1S)-1-cyclopropylethyl]carbamoyl}methyl)-2,5-dioxo-2′,3′-dihydrospiro[imidazolidine-4,1′-indene]-5′-yl]propanamide;
1-amino-N-[(4S)-1-({benzyl[(1S)-1-cyclopropylethyl]carbamoyl}methyl)-2,5-dioxo-2′,3′-dihydrospiro[imidazolidine-4, 1′-indene]-5′-yl]cyclopropane-1-carboxamide;
2-[(4S)-5′-{1-[2-(dimethylamino)-2-oxoethyl]-1H-pyrazol-4-yl}-2,5-dioxo-2,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(4S)-5′-{-1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-{(4S)-5′-[1-(2-amino-2-oxoethyl)-1H-pyrazol-4-yl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(3,4-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2 S)-1,1,1-trifluoropropan-2-yl]acetamide;
N-(4-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(4S)-5′-(1-methyl-1H-pyrazol-4-yl)-2,5-dioxo-2,3′-dihydro-1H-spiro[imidazolidine-4,1′-inden]-1-yl]-N-[(2S)-1,1,1-trifluoropropan-2-yl]acetamide;
N-(3-fluorobenzyl)-2-[(4S)-5′-(I-methyl-1H-pyrazol-4-yl)-2,5-dioxo-2′,3′-dihydro-1H-spiro[imidazolidine-4,1′-inden]-1-yl]-N-[(2 S)-1,1,1-trifluoropropan-2-yl]acetamide;
N-(2,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-[(1S)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-[(1S)-1-cyclopropyl-2,2,2-trifluoroethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-[(1R)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-benzyl-N-[(1S)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-benzyl-N-[(1R)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide;
2-[(4S)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4S)-2,5-dioxo-5′-(1H-pyrazol-4-yl)-2′,3′-dihydro-1H-spiro[imidazolidine-4,1′-inden]-1-yl]-N-(4-fluorobenzyl)-N-[(2S)-1,1, -trifluoropropan-2-yl]acetamide;
2-[(4S)-5′-amino-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4S)-5′-{[(2-cyanoethyl)carbamoyl]amino}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{(4S)-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
tert-butyl 4-[{[(4S)-5′-bromo-2,5-dioxo-2′,3′-dihydro-1H-spiro[imidazolidine-4,1′-inden]-1-yl]acetyl}(4-fluorobenzyl)amino]piperidine-1-carboxylate;
ethyl ({4-[{[(4S)-5′-amino-2,5-dioxo-2′,3′-dihydro-H-spiro[imidazolidine-4,1′-inden]-1-yl]acetyl}(4-fluorobenzyl)amino]piperidin-1-yl}sulfonyl)carbamate;
ethyl ({3,3-difluoro-4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]pyrrolidin-1-yl}sulfonyl)carbamate;
ethyl ({3,3-difluoro-4-[(4-fluorobenzyl) {[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]acetyl}amino]pyrrolidin-1-yl}sulfonyl)carbamate;
tert-butyl {4-[{[(4S)-5′-bromo-2,5-dioxo-2′,3′-dihydro-1H-spiro[imidazolidine-4,1′-inden]-1-yl]acetyl}(4-fluorobenzyl)amino]cyclohexylidene}acetate;
tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate;
{4-[{[(4S)-5′-amino-2,5-dioxo-2′,3′-dihydro-1H-spiro[imidazolidine-4,1′-inden]-1-yl]acetyl}(4-fluorobenzyl)amino]cyclohexylidene}acetic acid;
methyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate;
tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetate;
{4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetic acid;
2-[(4S)-2,5-dioxo-5′-(1H-pyrazol-5-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4S)-5′-{1-[2-(dimethylamino)ethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
tert-butyl 4-({4-[(4S)-1-(2-{(4-fluorobenzyl)[(2S)-1,1,1-trifluoropropan-2-yl]amino}-2-oxoethyl)-2,5-dioxo-2′,3′-dihydrospiro[imidazolidine-4,1′-inden]-5′-yl]-1H-pyrazol-1-yl}methyl)piperidine-1-carboxylate;
2-{(4S)-2,5-dioxo-5′-[1-(piperidin-4-ylmethyl)-1H-pyrazol-4-yl]-2′,3′-dihydro-1H-spiro[imidazolidine-4,1′-inden]-1-yl}-N-(4-fluorobenzyl)-N-[(2S)-1,1,1-trifluoropropan-2-yl]acetamide; and pharmaceutically acceptable salts thereof.

Still another embodiment pertains to compounds of Formula (XVa), selected from the group consisting of:
2-[(1R,3S)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R,3S)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide; and pharmaceutically acceptable salts thereof.

Still another embodiment pertains to compounds of Formula (XVb), selected from the group consisting of:
N-(4-fluorobenzyl)-2-[(3′R,4S)-3′-fluoro-2,5-dioxo-2′,3′-dihydro-1H-spiro[imidazolidine-4,1′-inden]-1-yl]-N-[(2S)-1,1, -trifluoropropan-2-yl]acetamide;
N-(4-fluorobenzyl)-2-[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(3′R,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropropan-2-yl]acetamide;
N-(4-fluorobenzyl)-2-[(3′R,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1 trifluoropropan-2-yl]acetamide;
2-[(3′R,4S)-5′-{1-[2-(dimethylamino)-2-oxoethyl]-1H-pyrazol-4-yl}-3-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3′R,4S)-5′-bromo-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{(3′R,4S)-5′-[1-(2-amino-2-oxoethyl)-H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-{(3′R,4S)-3′-fluoro-5′-[1-(2-hydroxyethyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(3′R,4S)-3′-fluoro-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
2-{(3′R,4S)-5′-[1-(difluoromethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]-N-(tetrahydro-2H-pyran-4-yl)acetamide;
{4-[(4-fluorobenzyl) {[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
4-[(4-fluorobenzyl) {[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexanecarboxylic acid; and pharmaceutically acceptable salts thereof.

In other particular embodiments of the compounds as described anywhere herein, the compound is a compound selected from one of the compounds in Table 1.

TABLE 1
Representative Compounds

| Example | Name |
|---|---|
| 2-1 | N-benzyl-N-(1-cyclobutylethyl)-2-(2,5-dioxo-2′,3′- |
| | dihydrospiro[imidazolidine-4,1′-indene]-1-yl)acetamide |
| 3-1 | N-benzyl-N-(1-cyclopropylethyl)-2-(2,5-dioxo-3′,4′-dihydro-2′H- |
| | spiro[imidazolidine-4,1′-naphthalene]-1-yl)acetamide |
| 4-1 | N-benzyl-N-(1-cyclopropylethyl)-2-(2′,5′-dioxospiro[chroman-4,4′- |
| | imidazolidine]-1′-yl)acetamide |
| 5-1 | N-benzyl-N-(1-cyclopropylethyl)-2-(2,2′,5-trioxospiro[imidazolidine-4,3′- |
| | indoline]-1-yl)acetamide |
| 6-1 | N-(4-bromobenzyl)-N-(1-cyclopropylethyl)-2-(2,5-dioxo-2′,3′- |
| | dihydrospiro[imidazolidine-4,1′-indene]-1-yl)acetamide |
| 7-1 | N-(1-cyclopropylethyl)-2-(2,5-dioxo-2′,3′-dihydrospiro[imidazolidine-4,1′- |
| | indene]-1-yl)-N-(furan-2-ylmethyl)acetamide |
| 8-1 | N-benzyl-N-(1-cyclopropylethyl)-2-(1′-methyl-2,2′,5- |
| | trioxospiro[imidazolidine-4,3′-indoline]-1-yl)acetamide |
| 9-1 | N-(1-cyclopropylethyl)-2-(2,5-dioxo-2′,3′-dihydrospiro[imidazolidine-4,1′- |
| | indene]-1-yl)-N-(thiophen-2-ylmethyl)acetamide |
| 10-1 | N-benzyl-N-(1-cyclopropylethyl)-2-(3-methyl-2,5-dioxo-2′,3′- |
| | dihydrospiro[imidazolidine-4,1′-indene]-1-yl)acetamide |
| 11-1 | N-(1-cyclopropylethyl)-2-(2,5-dioxo-2′,3′-dihydrospiro[imidazolidine-4,1′- |
| | indene]-1-yl)-N-(4-fluorobenzyl)acetamide |
| 12-1 | N-(1-cyclopropylethyl)-2-(2,5-dioxo-2′,3′-dihydrospiro[imidazolidine-4,1′- |
| | indene]-1-yl)-N-(2-fluorobenzyl)acetamide |
| 13-1 | N-(1-cyclopropylethyl)-2-(2,5-dioxo-2′,3′-dihydrospiro[imidazolidine-4,1′- |
| | indene]-1-yl)-N-(3-fluorobenzyl)acetamide |
| 14-1 | N-benzyl-2-(5′-bromo-2,5-dioxo-2′,3′-dihydrospiro[imidazolidine-4,1′- |
| | indene]-1-yl)-N-(1-cyclopropylethyl)acetamide |
| 15-1 | 2-(5′-bromo-2,5-dioxo-2′,3′-dihydrospiro[imidazolidine-4,1′-indene]-1-yl)- |
| | N-(1-cyclopropylethyl)-N-(furan-2-ylmethyl)acetamide |
| 16-1 | N-benzyl-N-(1-cyclopropylethyl)-2-(5′-fluoro-2,5-dioxo-2′,3′- |
| | dihydrospiro[imidazolidine-4,1′-indene]-1-yl)acetamide |
| 17-1 | N-benzyl-2-(4′-bromo-2,5-dioxo-2′,3′-dihydrospiro[imidazolidine-4,1′- |
| | indene]-1-yl)-N-(1-cyclopropylethyl)acetamide |
| 18-1 | N-(1-cyclopropylethyl)-2-(2,5-dioxo-2′,3′-dihydrospiro[imidazolidine-4,1′- |
| | indene]-1-yl)-N-(pyridin-3-ylmethyl)acetamide |
| 19-1 | N-(1-cyclopropylethyl)-2-(2,5-dioxo-2′,3′-dihydrospiro[imidazolidine-4,1′- |
| | indene]-1-yl)-N-(pyridin-2-ylmethyl)acetamide |
| 20-1 | N-benzyl-N-(1-cyclopropylethyl)-2-(5′-methoxy-2,5-dioxo-2′,3′- |
| | dihydrospiro[imidazolidine-4,1′-indene]-1-yl)acetamide |
| 21-1 | N-benzyl-N-(1-cyclopropylethyl)-2-(4′-methoxy-2,5-dioxo-2′,3′- |
| | dihydrospiro[imidazolidine-4,1′-indene]-1-yl)acetamide |
| 22-1 | N-benzyl-N-(1-cyclopropylethyl)-2-(2′,5′-dioxo-6,7- |
| | dihydrospiro[cyclopenta[b]pyridine-5,4′-imidazolidine]-1′-yl)acetamide |
| 23-1 | N-benzyl-2-(5′-cyano-2,5-dioxo-2′,3′-dihydrospiro[imidazolidine-4,1′- |
| | indene]-1-yl)-N-(1-cyclopropylethyl)acetamide |
| 24-1 | N-(1-cyclopropylethyl)-2-(2,5-dioxo-2′,3′-dihydrospiro[imidazolidine-4,1′- |
| | indene]-1-yl)-N-(pyridin-4-ylmethyl)acetamide |
| 25-1 | N-benzyl-2-(4′-chloro-2,5-dioxo-2′,3′-dihydrospiro[imidazolidine-4,1′- |
| | indene]-1-yl)-N-(1-cyclopropylethyl)acetamide |
| 26-1 | N-benzyl-2-(2,5-dioxo-2′,3′-dihydrospiro[imidazolidine-4,1′-indene]-1-yl)- |
| | N-isopropylacetamide |
| 27-1 | N-benzyl-N-sec-butyl-2-(2,5-dioxo-2′,3′-dihydrospiro[imidazolidine-4,1′- |
| | indene]-1-yl)acetamide |
| 28-1 | N-(1-cyclobutylethyl)-2-(2,5-dioxo-2′,3′-dihydrospiro[imidazolidine-4,1′- |
| | indene]-1-yl)-N-(4-fluorobenzyl)acetamide |
| 29-1 | N-benzyl-2-(4′-bromo-2,5-dioxo-2′,3′-dihydrospiro[imidazolidine-4,1′- |
| | indene]-1-yl)-N-((R)-1-cyclobutylethyl)acetamide |
| 30-1 | 2-(4′-bromo-2,5-dioxo-2′,3′-dihydrospiro[imidazolidine-4,1′-indene]-1-yl)- |
| | N-(1-cyclopropylethyl)-N-(4-fluorobenzyl)acetamide |
| 31-1 | N-benzyl-2-(5′-bromo-2,5-dioxo-2′,3′-dihydrospiro[imidazolidine-4,1′- |
| | indene]-1-yl)-N-(1-cyclobutylethyl)acetamide |
| 32-1 | 2-(5′-bromo-2,5-dioxo-2′,3′-dihydrospiro[imidazolidine-4,1′-indene]-1-yl)- |
| | N-(1-cyclopropylethyl)-N-(4-fluorobenzyl)acetamide |
| 33-1 | 1-(2-(benzyl(1-cyclopropylethyl)amino)-2-oxoethyl)-2,5-dioxo-2′,3′- |
| | dihydrospiro[imidazolidine-4,1′-indene]-5′-carboxamide |
| 34-1 | N-benzyl-2-(4′-cyano-2,5-dioxo-2′,3′-dihydrospiro[imidazolidine-4,1′- |
| | indene]-1-yl)-N-(1-cyclopropylethyl)acetamide |
| 35-1 | 1-(2-(benzyl(1-cyclopropylethyl)amino)-2-oxoethyl)-2,5-dioxo-2′,3′- |
| | dihydrospiro[imidazolidine-4,1′-indene]-4′-carboxamide |
| 36-1 | N-(1-cyclopropylethyl)-2-(2,5-dioxo-2′,3′-dihydrospiro[imidazolidine-4,1′- |
| | indene]-1-yl)-N-(naphthalen-2-ylmethyl)acetamide |
| 37-1 | N-((5-bromofuran-2-yl)methyl)-N-(1-cyclopropylethyl)-2-(2,5-dioxo-2′,3′- |
| | dihydrospiro[imidazolidine-4,1′-indene]-1-yl)acetamide |
| 38-1 | N-benzyl-N-((S)-1-cyclobutylethyl)-2-((S)-2,5-dioxo-2′,3′- |
| | dihydrospiro[imidazolidine-4,1′-indene]-1-yl)acetamide |
| 39-1 | N-benzyl-N-((R)-1-cyclobutylethyl)-2-((S)-2,5-dioxo-2′,3′- |
| | dihydrospiro[imidazolidine-4,1′-indene]-1-yl)acetamide |
| 40-1 | N-benzyl-N-(1-cyclopropylethyl)-2-(2′,5′-dioxo-5,6- |
| | dihydrospiro[cyclopenta[b]thiophene-4,4′-imidazolidine]-1′-yl)acetamide |
| 41-1 | N-benzyl-N-(1-cyclopropylethyl)-2-(2′,5′-dioxo-4,5- |
| | dihydrospiro[cyclopenta[b]thiophene-6,4′-imidazolidine]-1′-yl)acetamide |
| 42-1 | N-((1H-indol-5-yl)methyl)-N-(1-cyclopropylethyl)-2-(2,5-dioxo-2′,3′- |
| | dihydrospiro[imidazolidine-4,1′-indene]-1-yl)acetamide |
| 43-1 | N-benzyl-N-(1-cyclopropylethyl)-2-(2′,4′-dioxo-2,3-dihydrospiro[indene- |
| | 1,5′-ozazolidine]-3′-yl)acetamide |
| 44-1 | N-benzyl-2-(2,5-dioxo-2′,3′-dihydrospiro[imidazolidine-4,1′-indene]-1-yl)- |
| | N-(pentan-3-yl)acetamide |
| 45-1 | N-benzyl-N-(1-cyclopropylethyl)-2-(3′-methoxy-2,5-dioxo-2′,3′- |
| | dihydrospiro[imidazolidine-4,1′-indene]-1-yl)acetamide |
| 46-1 | N-benzyl-N-(1-cyclopropylethyl)-2-(5′-(methylthio)-2,5-dioxo-2′,3′- |
| | dihydrospiro[imidazolidine-4,1′-indene]-1-yl)acetamide |
| 47-1 | N-benzyl-2-(7′-chloro-2,5-dioxo-2′,3′-dihydrospiro[imidazolidine-4,1′- |
| | indene]-1-yl)-N-(1-cyclobutylethyl)acetamide |
| 48-1 | N-benzyl-N-(1-cyclopropylethyl)-2-(5′-(hydroxymethyl)-2,5-dioxo-2′,3′- |
| | dihydrospiro[imidazolidine-4,1′-indene]-1-yl)acetamide |
| 49-1 | N-benzyl-N-(1-cyclopropylethyl)-2-(5′-((difluoromethoxy)methyl)-2,5- |
| | dioxo-2′,3′-dihydrospiro[imidazolidine-4,1′-indene]-1-yl)acetamide |
| 50-1 | N-benzyl-2-(5′-cyclopropyl-2,5-dioxo-2′,3′-dihydrospiro[imidazolidine- |
| | 4,1′-indene]-1-yl)-N-(1-cyclopropylethyl)acetamide |
| 51-1 | N-benzyl-N-(1-cyclobutylethyl)-2-(3′-methyl-2,5-dioxo-2′,3′- |
| | dihydrospiro[imidazolidine-4,1′-indene]-1-yl)acetamide |
| 52-1 | N-benzyl-N-(1-cyclobutylethyl)-2-(5′-(difluoromethoxy)-2,5-dioxo-2′,3′- |
| | dihydrospiro[imidazolidine-4,1′-indene]-1-yl)acetamide |
| 53-1 | N-benzyl-N-(1-cyclopropylethyl)-2-(4′-methyl-2,5-dioxo-2′,3′- |
| | dihydrospiro[imidazolidine-4,1′-indene]-1-yl)acetamide |
| 54-1 | 2-(5′-acetamido-2,5-dioxo-2′,3′-dihydrospiro[imidazolidine-4,1′-indene]-1- |
| | yl)-N-benzyl-N-(1-cyclopropylethyl)acetamide |
| 55-1 | N-benzyl-N-(1-cyclopropylethyl)-2-(5′-methyl-2,5-dioxo-2′,3′- |
| | dihydrospiro[imidazolidine-4,1′-indene]-1-yl)acetamide |
| 56-1 | 1-(2-(benzyl((S)-1-cyclopropylethyl)amino)-2-oxoethyl)-N- |
| | (cyanomethyl)-2,5-dioxo-2′,3′-dihydrospiro[imidazolidine-4,1′-indene]-5′- |
| | carboxamide |
| 57-1 | N-benzyl-N-(1-cyclopropylethyl)-2-(5′-ethynyl-2,5-dioxo-2′,3′- |
| | dihydrospiro[imidazolidine-4,1′-indene]-1-yl)acetamide |
| 58-1 | N-benzyl-N-(1-cyclopropylethyl)-2-(5′-(3-hydroxyprop-1-ynyl)-2,5-dioxo- |
| | 2′,3′-dihydrospiro[imidazolidine-4,1′-indene]-1-yl)acetamide |
| 59-1 | N-benzyl-N-(1-cyclobutylethyl)-2-(6′-methoxy-2,5-dioxo-2′,3′- |
| | dihydrospiro[imidazolidine-4,1′-indene]-1-yl)acetamide |
| 60-1 | N-benzyl-N-(1-cyclobutylethyl)-2-(2-imino-5-oxo-2′,3′- |
| | dihydrospiro[imidazolidine-4,1′-indene]-1-yl)acetamide |
| 61-1 | N-benzyl-N-(dicyclopropylmethyl)-2-(2,5-dioxo-2′,3′- |
| | dihydrospiro[imidazolidine-4,1′-indene]-1-yl)acetamide |
| 62-1 | N-benzyl-2-(6′-chloro-2,5-dioxo-2′,3′-dihydrospiro[imidazolidine-4,1′- |
| | indene]-1-yl)-N-(1-cyclobutylethyl)acetamide |
| 63-1 | N-benzyl-N-(1-cyclopropylethyl)-2-(2,5-dioxo-5′-(1H-pyrazol-5-yl)-2′,3′- |
| | dihydrospiro[imidazolidine-4,1′-indene]-1-yl)acetamide |
| 64-1 | N-(4-chlorobenzyl)-N-((S)-1-cyclobutylethyl)-2-((S)-2,5-dioxo-2′,3′- |
| | dihydrospiro[imidazolidine-4,1′-indene]-1-yl)acetamide |
| 65-1 | N-benzyl-N-(1-cyclopropylethyl)-2-(2,5-dioxo-5′-(1H-1,2,3-triazol-4-yl)- |
| | 2′,3′-dihydrospiro[imidazolidine-4,1′-indene]-1-yl)acetamide |
| 66-1 | N-benzyl-N-((S)-1-cyclobutylethyl)-2-((3′R)-3′-hydroxy-2,5-dioxo-2′,3′- |
| | dihydrospiro[imidazolidine-4,1′-indene]-1-yl)acetamide |
| 67-1 | N-benzyl-N-((S)-1-cyclobutylethyl)-2-((3′S)-3′-hydroxy-2,5-dioxo-2′,3′- |
| | dihydrospiro[imidazolidine-4,1′-indene]-1-yl)acetamide |
| 68-1 | N-(4-bromobenzyl)-N-((S)-1-cyclobutylethyl)-2-((S)-2,5-dioxo-2′,3′- |
| | dihydrospiro[imidazolidine-4,1′-indene]-1-yl)acetamide |
| 69-1 | N-benzyl-N-(1-cyclopropylethyl)-2-(2,2′,5′-trioxo-1,2,6,7- |
| | tetrahydrospiro[cyclopenta[b]pyridine-5,4′-imidazolidine]-1′-yl)acetamide |
| 70-1 | 2-(5′-(1H-imidazol-2-yl)-2,5-dioxo-2′,3′-dihydrospiro[imidazolidine-4,1′- |
| | indene]-1-yl)-N-benzyl-N-(1-cyclopropylethyl)acetamide |
| 71-1 | N-benzyl-2-(5-bromo-2′,4′-dioxo-2,3-dihydrospiro[indene-1,5′- |
| | ozazolidine]-3′-yl)-N-(1-cyclopropylethyl)acetamide |
| 72-1 | N-benzyl-2-(2′,4′-dioxo-2,3-dihydrospiro[indene-1,5′-ozazolidine]-3′-yl)- |
| | N-(pentan-3-yl)acetamide |
| 73-1 | N-benzyl-N-(1-cyclobutylethyl)-2-(2′,4′-dioxo-2,3-dihydrospiro[indene- |
| | 1,5′-ozazolidine]-3′-yl)acetamide |
| 74-1 | N-(1-cyclopropylethyl)-2-(2′,4′-dioxo-2,3-dihydrospiro[indene-1,5′- |
| | ozazolidine]-3′-yl)-N-(4-fluorobenzyl)acetamide |
| 75-1 | N-benzyl-N-(1-cyclopropylethyl)-2-(4-methoxy-2′,4′-dioxo-2,3- |
| | dihydrospiro[indene-1,5′-ozazolidine]-3′-yl)acetamide |
| 76-1 | N-((S)-1-cyclobutylethyl)-2-((S)-2,5-dioxo-2′,3′- |
| | dihydrospiro[imidazolidine-4,1′-indene]-1-yl)-N-(4- |
| | (methylthio)benzyl)acetamide |
| 77-1 | N-benzyl-N-(1-cyclopropylethyl)-2-(2,5-dioxo-5′-(1H-1,2,3-triazol-1-yl)- |
| | 2′,3′-dihydrospiro[imidazolidine-4,1′-indene]-1-yl)acetamide |
| 77-2 | N-benzyl-N-(1-cyclopropylethyl)-2-(2,5-dioxo-5′-(2H-1,2,3-triazol-2-yl)- |
| | 2′,3′-dihydrospiro[imidazolidine-4,1′-indene]-1-yl)acetamide |
| 78-1 | N-((S)-1-cyclobutylethyl)-2-((S)-2,5-dioxo-2′,3′- |
| | dihydrospiro[imidazolidine-4,1′-indene]-1-yl)-N-(4- |
| | fluorobenzyl)acetamide |
| 79-1 | N-(4-cyanobenzyl)-N-((S)-1-cyclobutylethyl)-2-((S)-2,5-dioxo-2′,3′- |
| | dihydrospiro[imidazolidine-4,1′-indene]-1-yl)acetamide |
| 80-1 | 4-((N-((S)-1-cyclobutylethyl)-2-((S)-2,5-dioxo-2′,3′- |
| | dihydrospiro[imidazolidine-4,1′-indene]-1- |
| | yl)acetamido)methyl)benzamide |
| 81-1 | N-benzyl-N-(1-cyclopropylethyl)-2-(5′-(methoxymethyl)-2,5-dioxo-2′,3′- |
| | dihydrospiro[imidazolidine-4,1′-indene]-1-yl)acetamide |
| 82-1 | N-((S)-1-cyclobutylethyl)-2-((S)-2,5-dioxo-2′,3′- |
| | dihydrospiro[imidazolidine-4,1′-indene]-1-yl)-N-(4- |
| | methylbenzyl)acetamide |
| 83-1 | N-((S)-1-cyclobutylethyl)-2-((S)-2,5-dioxo-2′,3′- |
| | dihydrospiro[imidazolidine-4,1′-indene]-1-yl)-N-(4- |
| | methoxybenzyl)acetamide |
| 84-1 | N-((S)-1-cyclobutylethyl)-2-((S)-2,5-dioxo-2′,3′- |
| | dihydrospiro[imidazolidine-4,1′-indene]-1-yl)-N-(3- |
| | methylbenzyl)acetamide |
| 85-1 | N-((S)-1-cyclobutylethyl)-2-((S)-2,5-dioxo-2′,3′- |
| | dihydrospiro[imidazolidine-4,1′-indene]-1-yl)-N-(2- |
| | methylbenzyl)acetamide |
| 86-1 | N-((1H-1,2,3-triazol-4-yl)methyl)-N-((S)-1-cyclobutylethyl)-2-((S)-2,5- |
| | dioxo-2′,3′-dihydrospiro[imidazolidine-4,1′-indene]-1-yl)acetamide |
| 87-1 | N-((S)-1-cyclobutylethyl)-2-((S)-2,5-dioxo-2′,3′- |
| | dihydrospiro[imidazolidine-4,1′-indene]-1-yl)-N-(4- |
| | (methylsulfonyl)benzyl)acetamide |
| 88-1 | 2-(5-bromo-2′,4′-dioxo-2,3-dihydrospiro[indene-1,5′-ozazolidine]-3′-yl)-N- |
| | (1-cyclopropylethyl)-N-(4-fluorobenzyl)acetamide |
| 89-1 | N-benzyl-2-(5-cyano-2′,4′-dioxo-2,3-dihydrospiro[indene-1,5′- |
| | ozazolidine]-3′-yl)-N-(1-cyclopropylethyl)acetamide |
| 90-1 | N-benzyl-N-((S)-1-cyclopropylethyl)-2-(2,3′,5-trioxo-2′,3′- |
| | dihydrospiro[imidazolidine-4,1′-indene]-1-yl)acetamide |
| 91-1 | N-benzyl-2-(5′-(2-cyanoacetamido)-2,5-dioxo-2′,3′- |
| | dihydrospiro[imidazolidine-4,1′-indene]-1-yl)-N-((S)-1- |
| | cyclopropylethyl)acetamide |
| 92-1 | methyl 1-(2-(benzyl((S)-1-cyclopropylethyl)amino)-2-oxoethyl)-2,5- |
| | dioxo-2′,3′-dihydrospiro[imidazolidine-4,1′-indene]-5′-ylcarbamate |
| 93-1 | N-benzyl-N-((S)-1-cyclopropylethyl)-2-(7′-fluoro-2,5-dioxo-2′,3′- |
| | dihydrospiro[imidazolidine-4,1′-indene]-1-yl)acetamide |
| 94-1 | N-((S)-1-cyclobutylethyl)-2-((S)-2,5-dioxo-2′,3′- |
| | dihydrospiro[imidazolidine-4,1′-indene]-1-yl)-N-(1-phenylethyl)acetamide |
| 95-1 | N-benzyl-2-(7′-cyano-2,5-dioxo-2′,3′-dihydrospiro[imidazolidine-4,1′- |
| | indene]-1-yl)-N-((S)-1-cyclopropylethyl)acetamide |
| 96-1 | (S)-2-(5-acetamido-2′,4′-dioxo-2,3-dihydrospiro[indene-1,5′-ozazolidine]- |
| | 3′-yl)-N-benzyl-N-(1-cyclopropylethyl)acetamide |
| 97-1 | 3′-(2-(benzyl(1-cyclopropylethyl)amino)-2-oxoethyl)-2′,4′-dioxo-2,3- |
| | dihydrospiro[indene-1,5′-ozazolidine]-5-carboxamide |
| 98-1 | N-benzyl-N-((S)-1-cyclopropylethyl)-2-(5′-(3-methylureido)-2,5-dioxo- |
| | 2′,3′-dihydrospiro[imidazolidine-4,1′-indene]-1-yl)acetamide |
| 99-1 | N-benzyl-N-((S)-1-cyclopropylethyl)-2-(5′-(3-methyl-3- |
| | (methylcarbamoyl)ureido)-2,5-dioxo-2′,3′-dihydrospiro[imidazolidine-4,1′- |
| | indene]-1-yl)acetamide |
| 100-1 | N-benzyl-N-(1-cyclopropylethyl)-2-(5-methoxy-2′,4′-dioxo-2,3- |
| | dihydrospiro[indene-1,5′-ozazolidine]-3′-yl)acetamide |
| 101-1 | N-((S)-1-cyclopropylethyl)-2-((S)-2,5-dioxo-2′,3′- |
| | dihydrospiro[imidazolidine-4,1′-indene]-1-yl)-N-(4- |
| | ethynylbenzyl)acetamide |
| 102-1 | N-((S)-1-cyclobutylethyl)-2-((S)-2,5-dioxo-2′,3′- |
| | dihydrospiro[imidazolidine-4,1′-indene]-1-yl)-N-((1-methyl-1H-1,2,3- |
| | triazol-4-yl)methyl)acetamide |
| 103-1 | N-((1-(cyanomethyl)-1H-1,2,3-triazol-4-yl)methyl)-N-((S)-1- |
| | cyclobutylethyl)-2-((S)-2,5-dioxo-2′,3′-dihydrospiro[imidazolidine-4,1′- |
| | indene]-1-yl)acetamide |
| 104-1 | N-((S)-1-cyclobutylethyl)-2-((S)-2,5-dioxo-2′,3′- |
| | dihydrospiro[imidazolidine-4,1′-indene]-1-yl)-N-(4- |
| | ureidobenzyl)acetamide |
| 105-1 | N-benzyl-N-((S)-1-cyclopropylethyl)-2-(5-(3-hydroxyprop-1-ynyl)-2′,4′- |
| | dioxo-2,3-dihydrospiro[indene-1,5′-ozazolidine]-3′-yl)acetamide |
| 106-1 | N-benzyl-N-((S)-1-cyclopropylethyl)-2-(5′-(3-hydroxy-3-methylbut-1- |
| | ynyl)-2,5-dioxo-2′,3′-dihydrospiro[imidazolidine-4,1′-indene]-1- |
| | yl)acetamide |
| 107-1 | N-((1-(2-amino-2-oxoethyl)-1H-1,2,3-triazol-4-yl)methyl)-N-((S)-1- |
| | cyclobutylethyl)-2-((S)-2,5-dioxo-2′,3′-dihydrospiro[imidazolidine-4,1′- |
| | indene]-1-yl)acetamide |
| 108-1 | (S)-N-benzyl-N-(1-cyclopropylethyl)-2-(2,5-dioxo-5′-(6-oxo-1,6- |
| | dihydropyridin-3-yl)-2′,3′-dihydrospiro[imidazolidine-4,1′-indene]-1- |
| | yl)acetamide |
| 109-1 | N-benzyl-N-((S)-1-cyclopropylethyl)-2-((S)-2,5-dioxo-5′-(1H-pyrazol-3- |
| | yl)-2′,3′-dihydrospiro[imidazolidine-4,1′-indene]-1-yl)acetamide |
| 110-1 | N-benzyl-N-((S)-1-cyclopropylethyl)-2-(5-(methylsulfonamido)-2′,4′- |
| | dioxo-2,3-dihydrospiro[indene-1,5′-ozazolidine]-3′-yl)acetamide |
| 111-1 | N-benzyl-N-((S)-1-cyclopropylethyl)-2-(5′-(methylsulfonamido)-2,5- |
| | dioxo-2′,3′-dihydrospiro[imidazolidine-4,1′-indene]-1-yl)acetamide |
| 112-1 | N-benzyl-2-(5′-(2-cyanoacetamido)-2,5-dioxo-2′,3′- |
| | dihydrospiro[imidazolidine-4,1′-indene]-1-yl)-N-((S)-1- |
| | cyclopropylethyl)acetamide |
| 113-1 | N-benzyl-N-((S)-1-cyclopropylethyl)-2-(2,5-dioxo-5′-(2H-tetrazol-5-yl)- |
| | 2′,3′-dihydrospiro[imidazolidine-4,1′-indene]-1-yl)acetamide |
| 114-1 | (S)-N-benzyl-N-(1-cyclopropylethyl)-2-(3′-(hydroxyimino)-2,5-dioxo- |
| | 2′,3′-dihydrospiro[imidazolidine-4,1′-indene]-1-yl)acetamide |
| 115-1 | (S)-N-benzyl-N-(1-cyclopropylethyl)-2-(3′-(methoxyimino)-2,5-dioxo- |
| | 2′,3′-dihydrospiro[imidazolidine-4,1′-indene]-1-yl)acetamide |
| 116-1 | (S)-3′-(2-(benzyl(1-cyclopropylethyl)amino)-2-oxoethyl)-2′,4′-dioxo-2,3- |
| | dihydrospiro[indene-1,5′-ozazolidine]-5-carboxamide |
| 117-1 | N-benzyl-N-((S)-1-cyclopropylethyl)-2-((S)-2,5-dioxo-5′-(6-oxo-1,6- |
| | dihydropyridin-3-yl)-2′,3′-dihydrospiro[imidazolidine-4,1′-indene]-1- |
| | yl)acetamide |
| 118-1 | N-benzyl-N-((S)-1-cyclopropylethyl)-2-((S)-5′-(3-hydroxyprop-1-ynyl)- |
| | 2,5-dioxo-2′,3′-dihydrospiro[imidazolidine-4,1′-indene]-1-yl)acetamide |
| 119-1 | N-benzyl-N-((S)-1-cyclopropylethyl)-2-((S)-2,5-dioxo-5′-(2H-1,2,3- |
| | triazol-4-yl)-2′,3′-dihydrospiro[imidazolidine-4,1′-indene]-1-yl)acetamide |
| 120-1 | N-benzyl-N-((S)-1-cyclopropylethyl)-2-(5-(morpholinomethyl)-2′,4′- |
| | dioxo-2,3-dihydrospiro[indene-1,5′-ozazolidine]-3′-yl)acetamide |
| 121-1 | N-benzyl-N-((S)-1-cyclopropylethyl)-2-(5-(3-methylureido)-2′,4′-dioxo- |
| | 2,3-dihydrospiro[indene-1,5′-ozazolidine]-3′-yl)acetamide |
| 122-1 | N-benzyl-N-((S)-1-cyclopropylethyl)-2-(5′-(3-hydroxypyrrolidin-1-yl)- |
| | 2,5-dioxo-2′,3′-dihydrospiro[imidazolidine-4,1′-indene]-1-yl)acetamide |
| 123-1 | N-benzyl-N-((S)-1-cyclopropylethyl)-2-((S)-5′-(3-hydroxy-3-methylbut-1- |
| | ynyl)-2,5-dioxo-2′,3′-dihydrospiro[imidazolidine-4,1′-indene]-1- |
| | yl)acetamide |
| 124-1 | (S)-N-(3′-(2-(benzyl(1-cyclopropylethyl)amino)-2-oxoethyl)-2′,4′-dioxo- |
| | 2,3-dihydrospiro[indene-1,5′-ozazolidine]-5-yl)-3-oxobutanamide |
| 125-1 | N-benzyl-N-((S)-1-cyclopropylethyl)-2-(5-morpholino-2′,4′-dioxo-2,3- |
| | dihydrospiro[indene-1,5′-ozazolidine]-3′-yl)acetamide |
| 126-1 | N-benzyl-2-(5-(2-cyanoacetamido)-2′,4′-dioxo-2,3-dihydrospiro[indene- |
| | 1,5′-ozazolidine]-3′-yl)-N-((S)-1-cyclopropylethyl)acetamide |
| 127-1 | N-benzyl-2-((S)-5′-cyano-2,5-dioxo-2′,3′-dihydrospiro[imidazolidine-4,1′- |
| | indene]-1-yl)-N-((S)-1-cyclopropylethyl)acetamide |
| 128-1 | 1-(2-(benzyl((S)-1-cyclopropylethyl)amino)-2-oxoethyl)-2,5-dioxo-2′,3′- |
| | dihydrospiro[imidazolidine-4,1′-indene]-3′-carboxylic acid |
| 129-1 | N-benzyl-N-((S)-1-cyclopropylethyl)-2-((S)-5′-(3-methylureido)-2,5- |
| | dioxo-2′,3′-dihydrospiro[imidazolidine-4,1′-indene]-1-yl)acetamide |
| 130-1 | N-benzyl-N-((S)-1-cyclopropylethyl)-2-(2′,4′-dioxo-5-(2-oxa-6- |
| | azaspiro[3.3]heptan-6-yl)-2,3-dihydrospiro[indene-1,5′-ozazolidine]-3′- |
| | yl)acetamide |
| 131-1 | N-benzyl-N-((S)-1-cyclopropylethyl)-2-(2′,4′-dioxo-5-(piperidin-1- |
| | ylmethyl)-2,3-dihydrospiro[indene-1,5′-ozazolidine]-3′-yl)acetamide |
| 132-1 | N-benzyl-N-((S)-1-cyclopropylethyl)-2-(2′,4′-dioxo-5-(piperazin-1- |
| | ylmethyl)-2,3-dihydrospiro[indene-1,5′-ozazolidine]-3′-yl)acetamide |
| 133-1 | N-benzyl-N-((S)-1-cyclopropylethyl)-2-(5-((4-hydroxypiperidin-1- |
| | yl)methyl)-2′,4′-dioxo-2,3-dihydrospiro[indene-1,5′-ozazolidine]-3′- |
| | yl)acetamide |
| 134-1 | (S)-2-amino-N-(3′-(2-(benzyl(1-cyclopropylethyl)amino)-2-oxoethyl)- |
| | 2′,4′-dioxo-2,3-dihydrospiro[indene-1,5′-ozazolidine]-5-yl)-2- |
| | methylpropanamide |
| 135-1 | (S)-N-benzyl-N-(1-cyclopropylethyl)-2-(2′,4′-dioxo-5-(piperazin-1-yl)-2,3- |
| | dihydrospiro[indene-1,5′-ozazolidine]-3′-yl)acetamide |
| 136-1 | N-benzyl-N-((S)-1-cyclopropylethyl)-2-(5′-(3-hydroxyazetidin-1-yl)-2,5- |
| | dioxo-2′,3′-dihydrospiro[imidazolidine-4,1′-indene]-1-yl)acetamide |
| 137-1 | N-benzyl-2-((S)-5′-(2-cyanoacetamido)-2,5-dioxo-2′,3′- |
| | dihydrospiro[imidazolidine-4,1′-indene]-1-yl)-N-((S)-1- |
| | cyclopropylethyl)acetamide |
| 138-1 | 1-(2-(benzyl((S)-1-cyclopropylethyl)amino)-2-oxoethyl)-2,5-dioxo-2′,3′- |
| | dihydrospiro[imidazolidine-4,1′-indene]-3′-carboxamide |
| 139-1 | N-benzyl-2-(3′-cyano-2,5-dioxo-2′,3′-dihydrospiro[imidazolidine-4,1′- |
| | indene]-1-yl)-N-((S)-1-cyclopropylethyl)acetamide |
| 140-1 | N-benzyl-N-((S)-1-cyclopropylethyl)-2-(5′-(2,4-dioxo-3,4- |
| | dihydropyrimidin-1(2H)-yl)-2,5-dioxo-2′,3′-dihydrospiro[imidazolidine- |
| | 4,1′-indene]-1-yl)acetamide |
| 141-1 | N-benzyl-N-((S)-1-cyclopropylethyl)-2-(5-hydroxy-2′,4′-dioxo-2,3- |
| | dihydrospiro[indene-1,5′-ozazolidine]-3′-yl)acetamide |
| 142-1 | methyl 2-(3′-(2-(benzyl((S)-1-cyclopropylethyl)amino)-2-oxoethyl)-2′,4′- |
| | dioxo-2,3-dihydrospiro[indene-1,5′-ozazolidine]-5-yloxy)acetate |
| 143-1 | N-benzyl-N-((S)-1-cyclopropylethyl)-2-(2′,4′-dioxo-5-(prop-2-ynyloxy)- |
| | 2,3-dihydrospiro[indene-1,5′-ozazolidine]-3′-yl)acetamide |
| 144-1 | N-(1-(2-(benzyl((S)-1-cyclopropylethyl)amino)-2-oxoethyl)-2,5-dioxo- |
| | 2′,3′-dihydrospiro[imidazolidine-4,1′-indene]-5′-yl)acrylamide |
| 145-1 | N-benzyl-2-(5′-(2-cyano-1-hydroxyallyl)-2,5-dioxo-2′,3′- |
| | dihydrospiro[imidazolidine-4,1′-indene]-1-yl)-N-((S)-1- |
| | cyclopropylethyl)acetamide |
| 146-1 | (S)-2-amino-N-(3′-(2-(benzyl((S)-1-cyclopropylethyl)amino)-2-oxoethyl)- |
| | 2′,4′-dioxo-2,3-dihydrospiro[indene-1,5′-ozazolidine]-5-yl)-3- |
| | methylbutanamide |
| 147-1 | (2S)-2-amino-N-(3′-(2-(benzyl((S)-1-cyclopropylethyl)amino)-2- |
| | oxoethyl)-2′,4′-dioxo-2,3-dihydrospiro[indene-1,5′-ozazolidine]-5- |
| | yl)propanamide |
| 148-1 | N-benzyl-N-((S)-1-cyclopropylethyl)-2-(5′-(2,4-dioxo-1,2,3,4- |
| | tetrahydropyrimidin-5-yl)-2,5-dioxo-2′,3′-dihydrospiro[imidazolidine-4,1′- |
| | indene]-1-yl)acetamide |
| 149-1 | 1-(2-(benzyl((S)-1-cyclopropylethyl)amino)-2-oxoethyl)-2,5-dioxo-2′,3′- |
| | dihydrospiro[imidazolidine-4,1′-indene]-5′-carboxylic acid |
| 150-1 | 2-(3′-(2-(benzyl((S)-1-cyclopropylethyl)amino)-2-oxoethyl)-2′,4′-dioxo- |
| | 2,3-dihydrospiro[indene-1,5′-ozazolidine]-5-ylamino)acetic acid |
| 151-1 | N-(3′-(2-(benzyl((S)-1-cyclopropylethyl)amino)-2-oxoethyl)-2′,4′-dioxo- |
| | 2,3-dihydrospiro[indene-1,5′-ozazolidine]-5-yl)-2-cyano-3-oxobutanamide |
| 152-1 | (S)-N-(1-(2-(benzyl(1-cyclopropylethyl)amino)-2-oxoethyl)-2,5-dioxo- |
| | 2′,3′-dihydrospiro[imidazolidine-4,1′-indene]-5′-yl)-3-oxobutanamide |
| 153-1 | N-(1-(2-(benzyl((S)-1-cyclopropylethyl)amino)-2-oxoethyl)-2,5-dioxo- |
| | 2′,3′-dihydrospiro[imidazolidine-4,1′-indene]-5′-yl)-2-cyano-3- |
| | oxobutanamide |
| 154-1 | 2-(3′-(2-(benzyl((S)-1-cyclopropylethyl)amino)-2-oxoethyl)-2′,4′-dioxo- |
| | 2,3-dihydrospiro[indene-1,5′-ozazolidine]-5-yloxy)acetic acid |
| 155-1 | N-benzyl-N-((S)-1-cyclopropylethyl)-2-(5-(oxetan-3-ylamino)-2′,4′-dioxo- |
| | 2,3-dihydrospiro[indene-1,5′-ozazolidine]-3′-yl)acetamide |
| 156-1 | N-benzyl-N-((S)-1-cyclopropylethyl)-2-(2′,4′-dioxo-5-(tetrahydro-2H- |
| | pyran-4-ylamino)-2,3-dihydrospiro[indene-1,5′-ozazolidine]-3′- |
| | yl)acetamide |
| 157-1 | N-(3′-(2-(benzyl((S)-1-cyclopropylethyl)amino)-2-oxoethyl)-2′,4′-dioxo- |
| | 2,3-dihydrospiro[indene-1,5′-ozazolidine]-5-yl)-2-hydroxypropanamide |
| 158-1 | (E)-N-(1-(2-(benzyl((S)-1-cyclopropylethyl)amino)-2-oxoethyl)-2,5- |
| | dioxo-2′,3′-dihydrospiro[imidazolidine-4,1′-indene]-5′-yl)-4-(piperidin-1- |
| | yl)but-2-enamide |
| 159-1 | N-benzyl-2-((S)-2,5-dioxo-2′,3′-dihydrospiro[imidazolidine-4,1′-indene]- |
| | 1-yl)-N-(1-(oxetan-3-yl)ethyl)acetamide |
| 160-1 | 1-(3′-(2-(benzyl((S)-1-cyclopropylethyl)amino)-2-oxoethyl)-2′,4′-dioxo- |
| | 2,3-dihydrospiro[indene-1,5′-ozazolidine]-5-yl)azetidine-3-carboxamide |
| 161-1 | 2-amino-N-(3′-(2-(benzyl(dicyclopropylmethyl)amino)-2-oxoethyl)-2′,4′- |
| | dioxo-2,3-dihydrospiro[indene-1,5′-ozazolidine]-5-yl)-2- |
| | methylpropanamide |
| 162-1 | N-benzyl-N-(dicyclopropylmethyl)-2-(5-(3-methylureido)-2′,4′-dioxo-2,3- |
| | dihydrospiro[indene-1,5′-ozazolidine]-3′-yl)acetamide |
| 163-1 | N-benzyl-N-((S)-1-cyclopropylethyl)-2-(5-(2-(dimethylamino)ethoxy)- |
| | 2′,4′-dioxo-2,3-dihydrospiro[indene-1,5′-ozazolidine]-3′-yl)acetamide |
| 164-1 | 2-(5-((1H-1,2,3-triazol-4-yl)methoxy)-2′,4′-dioxo-2,3- |
| | dihydrospiro[indene-1,5′-ozazolidine]-3′-yl)-N-benzyl-N-((S)-1- |
| | cyclopropylethyl)acetamide |
| 165-1 | N-benzyl-N-((S)-1-cyclopropylethyl)-2-(2′,4′-dioxo-5-(6-oxo-1,6- |
| | dihydropyridin-3-yl)-2,3-dihydrospiro[indene-1,5′-ozazolidine]-3′- |
| | yl)acetamide |
| 166-1 | N-benzyl-N-((S)-1-cyclopropylethyl)-2-(5-(2-hydroxyethoxy)-2′,4′-dioxo- |
| | 2,3-dihydrospiro[indene-1,5′-ozazolidine]-3′-yl)acetamide |
| 167-1 | 3′-(2-(benzyl((S)-1-cyclopropylethyl)amino)-2-oxoethyl)-2′,4′-dioxo-2,3- |
| | dihydrospiro[indene-1,5′-ozazolidine]-5-ylboronic acid |
| 168-1 | N-benzyl-N-((S)-1-cyclopropylethyl)-2-(5-(3-(hydroxymethyl)azetidin-1- |
| | yl)-2′,4′-dioxo-2,3-dihydrospiro[indene-1,5′-ozazolidine]-3′-yl)acetamide |
| 169-1 | 2-amino-N-((1′S)-1-((4S)-3-benzyl-4-cyclopropyl-2-oxopentyl)-2,5-dioxo- |
| | 2′,3′-dihydrospiro[imidazolidine-4,1′-indene]-5′-yl)-2-methylpropanamide |
| 170-1 | N-((S)-1-cyclopropylethyl)-2-(2′,4′-dioxo-5-(6-oxo-1,6-dihydropyridin-3- |
| | yl)-2,3-dihydrospiro[indene-1,5′-ozazolidine]-3′-yl)-N-(4- |
| | fluorobenzyl)acetamide |
| 171-1 | (S)-2-amino-N-((S)-1-(2-(benzyl((S)-1-cyclopropylethyl)amino)-2- |
| | oxoethyl)-2,5-dioxo-2′,3′-dihydrospiro[imidazolidine-4,1′-indene]-5′- |
| | yl)propanamide |
| 172-1 | 1-amino-N-(3′-(2-(benzyl((S)-1-cyclopropylethyl)amino)-2-oxoethyl)- |
| | 2′,4′-dioxo-2,3-dihydrospiro[indene-1,5′-ozazolidine]-5- |
| | yl)cyclobutanecarboxamide |
| 173-1 | N-benzyl-N-((S)-1-cyclopropylethyl)-2-(5′-(2-hydroxy-5-oxo-2,5- |
| | dihydrofuran-2-yl)-2,5-dioxo-2′,3′-dihydrospiro[imidazolidine-4,1′- |
| | indene]-1-yl)acetamide |
| 174-1 | 2-(5-(2-cyanoacetamido)-2′,4′-dioxo-2,3-dihydrospiro[indene-1,5′- |
| | ozazolidine]-3′-yl)-N-((S)-1-cyclopropylethyl)-N-(4- |
| | fluorobenzyl)acetamide |
| 175-1 | N-((S)-1-cyclopropylethyl)-N-(4-fluorobenzyl)-2-(5-(3-methylureido)- |
| | 2′,4′-dioxo-2,3-dihydrospiro[indene-1,5′-ozazolidine]-3′-yl)acetamide |
| 176-1 | 5′-(azetidin-3-ylmethylamino)-1-((4S)-3-benzyl-4-cyclopropyl-2- |
| | oxopentyl)-2′,3′-dihydrospiro[imidazolidine-4,1′-indene]-2,5-dione |
| 177-1 | 3′-((4S)-3-benzyl-4-cyclopropyl-2-oxopentyl)-5-(1H-pyrazol-5-yl)-2,3- |
| | dihydrospiro[indene-1,5′-ozazolidine]-2′,4′-dione |
| 178-1 | 2-amino-N-(3′-(2-(benzyl((S)-1-cyclopropylethyl)amino)-2-oxoethyl)-4′- |
| | oxo-2,3-dihydrospiro[indene-1,5′-ozazolidine]-5-yl)-3- |
| | hydroxypropanamide |
| 179-1 | (S)-N-(3′-(2-(benzyl(1-cyclopropylethyl)amino)-2-oxoethyl)-2′,4′-dioxo- |
| | 2,3-dihydrospiro[indene-1,5′-ozazolidine]-5-yl)-2-hydroxy-2- |
| | methylpropanamide |
| 180-1 | 1-amino-N-(3′-(2-(benzyl((S)-1-cyclopropylethyl)amino)-2-oxoethyl)- |
| | 2′,4′-dioxo-2,3-dihydrospiro[indene-1,5′-ozazolidine]-5- |
| | yl)cyclopropanecarboxamide |
| 181-1 | 2-amino-N-(3′-(2-(((S)-1-cyclopropylethyl)(4-fluorobenzyl)amino)-2- |
| | oxoethyl)-2′,4′-dioxo-2,3-dihydrospiro[indene-1,5′-ozazolidine]-5-yl)-2- |
| | methylpropanamide |
| 182-1 | (S)-2-(5-(azetidin-3-ylamino)-2′,4′-dioxo-2,3-dihydrospiro[indene-1,5′- |
| | ozazolidine]-3′-yl)-N-benzyl-N-(1-cyclopropylethyl)acetamide |
| 183-1 | 1-amino-N-((S)-1-(2-(benzyl((S)-1-cyclopropylethyl)amino)-2-oxoethyl)- |
| | 2,5-dioxo-2′,3′-dihydrospiro[imidazolidine-4,1′-indene]-5′- |
| | yl)cyclobutanecarboxamide |
| 184-1 | (2R)-2-amino-N-(3′-(2-(benzyl((S)-1-cyclopropylethyl)amino)-2- |
| | oxoethyl)-2′,4′-dioxo-2,3-dihydrospiro[indene-1,5′-ozazolidine]-5- |
| | yl)propanamide |
| 185-1 | N-benzyl-N-((S)-1-cyclopropylethyl)-2-(2,5-dioxo-5′-(thiazol-2-ylamino)- |
| | 2′,3′-dihydrospiro[imidazolidine-4,1′-indene]-1-yl)acetamide |
| 186-1 | (S)-2-(5′-(azetidin-3-ylamino)-2,5-dioxo-2′,3′-dihydrospiro[imidazolidine- |
| | 4,1′-indene]-1-yl)-N-benzyl-N-(1-cyclopropylethyl)acetamide |
| 187-1 | (S)-2-(3′-(2-(benzyl(1-cyclopropylethyl)amino)-2-oxoethyl)-2′,4′-dioxo- |
| | 2,3-dihydrospiro[indene-1,5′-ozazolidine]-5-ylcarboxamido)acetic acid |
| 188-1 | 2-((S)-5-acetamido-2′,4′-dioxo-2,3-dihydrospiro[indene-1,5′-ozazolidine]- |
| | 3′-yl)-N-benzyl-N-((S)-1-cyclopropylethyl)acetamide |
| 189-1 | 2-((R)-5-acetamido-2′,4′-dioxo-2,3-dihydrospiro[indene-1,5′-ozazolidine]- |
| | 3′-yl)-N-benzyl-N-((S)-1-cyclopropylethyl)acetamide |
| 190-1 | N-benzyl-N-(1-cyclopropylethyl)-2-(2,5-dioxo-1′,3′- |
| | dihydrospiro[imidazolidine-4,2′-indene]-1-yl)acetamide |
| 191-1 | N-benzyl-N-(1-cyclopropylethyl)-2-(8′-methoxy-2,5-dioxo-3′,4′-dihydro- |
| | 1′H-spiro[imidazolidine-4,2′-naphthalene]-1-yl)acetamide |
| 192-1 | N-benzyl-N-(1-cyclobutylethyl)-2-(3′,3′-dimethyl-2,5-dioxo-2′,3′- |
| | dihydrospiro[imidazolidine-4,1′-indene]-1-yl)acetamide |
| 193-1 | N-benzyl-2-[5′-(5-cyano-6-oxo-1,6-dihydropyridin-3-yl)-2,5-dioxo-2′,3′- |
| | dihydrospiro[imidazolidine-4,1′-indene]-1-yl]-N-(1- |
| | cyclopropylethyl)acetamide |
| 194-1 | N-benzyl-2-{5-[(methylcarbamoyl)amino]-3′,5′-dioxo-2,3- |
| | dihydrospiro[indene-1,2′-[1,4]ozazolidine]-4′-yl}-N-(pentan-3- |
| | yl)acetamide |
| 194-2 | 2-amino-N-(4′-{[benzyl(pentan-3-yl)carbamoyl]methyl}-3′,5′-dioxo-2,3- |
| | dihydrospiro[indene-1,2′-[1,4]ozazolidine]-5-yl)-2-methylpropanamide |
| 195-1 | (2R)-2-amino-N-[(4S)-1-({benzyl[(1S)-1- |
| | cyclopropylethyl]carbamoyl}methyl)-2,5-dioxo-2′,3′- |
| | dihydrospiro[imidazolidine-4,1′-indene]-5′-yl]propanamide |
| 196-1 | N-benzyl-N-[(1S)-1-cyclopropylethyl]-2-{5′-[(1H-imidazol-2-yl)amino]- |
| | 2,5-dioxo-2′,3′-dihydrospiro[imidazolidine-4,1′-indene]-1-yl}acetamide |
| 197-1 | 1-amino-N-[(4S)-1-({benzyl[(1S)-1-cyclopropylethyl]carbamoyl}methyl)- |
| | 2,5-dioxo-2′,3′-dihydrospiro[imidazolidine-4,1′-indene]-5′- |
| | yl]cyclopropane-1-carboxamide |
| 198-1 | 2-{5-[(azetidin-3-ylmethyl)amino]-3′,5′-dioxo-2,3-dihydrospiro[indene- |
| | 1,2′-[1,4]ozazolidine]-4′-yl}-N-benzyl-N-[(1S)-1- |
| | cyclopropylethyl]acetamide |
| 199-1 | N-benzyl-N-[(1R)-1-cyclopropyl-2,2,2-trifluoroethyl]-2-[(4S)-2,5-dioxo- |
| | 2′,3′-dihydrospiro[imidazolidine-4,1′-indene]-1-yl]acetamide |
| 200-1 | N-benzyl-N-[(1S)-1-cyclopropylethyl]-2-{5′-[(oxetan-3-ylmethyl)amino]- |
| | 2,5-dioxo-2′,3′-dihydrospiro[imidazolidine-4,1′-indene]-1-yl}acetamide |
| 201-1 | N-benzyl-N-[(1S)-1-cyclopropylethyl]-2-{5-[(1,3-dihydroxypropan-2- |
| | yl)oxy]-3′,5′-dioxo-2,3-dihydrospiro[indene-1,2′-[1,4]ozazolidine]-4′- |
| | yl}acetamide |
| 202-1 | 1-amino-N-[4′-({[(1S)-1-cyclopropylethyl][(4- |
| | fluorophenyl)methyl]carbamoyl}methyl)-3′,5′-dioxo-2,3- |
| | dihydrospiro[indene-1,2′-[1,4]ozazolidine]-5-yl]cyclobutane-1- |
| | carboxamide |
| 203-1 | N-[4′-({benzyl[(1S)-1-cyclopropylethyl]carbamoyl}methyl)-3′,5′-dioxo- |
| | 2,3-dihydrospiro[indene-1,2′-[1,4]ozazolidine]-5-yl]azetidine-3- |
| | carboxamide |
| 204-1 | 4-{[4′-({benzyl[(1S)-1-cyclopropylethyl]carbamoyl}methyl)-3′,5′-dioxo- |
| | 2,3-dihydrospiro[indene-1,2′-[1,4]ozazolidine]-5-ylmethyl]amino}benzoic |
| | acid |
| 205-1 | N-[4′-({benzyl[(1S)-1-cyclopropylethyl]carbamoyl}methyl)-3′,5′-dioxo- |
| | 2,3-dihydrospiro[indene-1,2′-[1,4]ozazolidine]-5-yl]-2,3- |
| | dihydroxypropanamide |
| 206-1 | N-benzyl-2-{5-[(methylcarbamoyl)amino]-3′,5′-dioxo-2,3- |
| | dihydrospiro[indene-1,2′-[1,4]ozazolidine]-4′-yl}-N-(oxolan-3- |
| | yl)acetamide |
| 207-1 | N-benzyl-N-[(1S)-1-cyclopropylethyl]-2-{5-[(1,1-dioxo-1λ⁶-thian-4- |
| | yl)amino]-3′,5′-dioxo-2,3-dihydrospiro[indene-1,2′-[1,4]ozazolidine]-4′- |
| | yl}acetamide |
| 208-1 | N-benzyl-N-[(1S)-1-cyclopropylethyl]-2-{1,1,2′,5′-tetraoxo-2H-spiro[1λ⁶- |
| | benzothiophene-3,4′-imidazolidine]-1′-yl}acetamide |
| 209-1 | 2-{5-[(2R)-2-amino-2-cyclopropylacetamido]-3′,5′-dioxo-2,3- |
| | dihydrospiro[indene-1,2′-[1,4]ozazolidine]-4′-yl}-N-[(1S)-1- |
| | cyclopropylethyl]-N-[(4-fluorophenyl)methyl]acetamide |
| 210-1 | 2-[5′-(azetidin-3-yloxy)-2,5-dioxo-2′,3′-dihydrospiro[imidazolidine-4,1′- |
| | indene]-1-yl]-N-benzyl-N-[(1S)-1-cyclopropylethyl]acetamide |
| 211-1 | N-(cyclopropylmethyl)-2-{5-[(methylcarbamoyl)amino]-3′,5′-dioxo-2,3- |
| | dihydrospiro[indene-1,2′-[1,4]ozazolidine]-4′-yl}-N-[(2- |
| | methylphenyl)methyl]acetamide |
| 212-1 | N-ethyl-2-{5-[(methylcarbamoyl)amino]-3′,5′-dioxo-2,3- |
| | dihydrospiro[indene-1,2′-[1,4]ozazolidine]-4′-yl}-N-[(2- |
| | methylphenyl)methyl]acetamide |
| 213-1 | N-[(1S)-1-cyclopropylethyl]-2-{3′,5′-dioxo-5-[(pyrrolidin-3-yl)amino]- |
| | 2,3-dihydrospiro[indene-1,2′-[1,4]ozazolidine]-4′-yl}-N-[(4- |
| | fluorophenyl)methyl]acetamide |
| 214-1 | 2-(5-{[(1-aminocyclopropyl)methyl]amino}-3′,5′-dioxo-2,3- |
| | dihydrospiro[indene-1,2′-[1,4]ozazolidine]-4′-yl)-N-[(1S)-1- |
| | cyclopropylethyl]-N-[(4-fluorophenyl)methyl]acetamide |
| 215-1 | N-benzyl-2-{5-[(methylcarbamoyl)amino]-3′,5′-dioxo-2,3- |
| | dihydrospiro[indene-1,2′-[1,4]ozazolidine]-4′-yl}-N-(oxan-4-yl)acetamide |
| 216-1 | N-benzyl-N-[(1R)-1-cyclopropyl-2,2,2-trifluoroethyl]-2-{5- |
| | [(methylcarbamoyl)amino]-3′,5′-dioxo-2,3-dihydrospiro[indene-1,2′- |
| | [1,4]ozazolidine]-4′-yl}acetamide |
| 217-1 | N-benzyl-2-{5-[(methylcarbamoyl)amino]-3′,5′-dioxo-2,3- |
| | dihydrospiro[indene-1,2′-[1,4]ozazolidine]-4′-yl}-N-(oxan-3-yl)acetamide |
| 218-1 | N-[4′-({benzyl[(1S)-1-cyclopropylethyl]carbamoyl}methyl)-3′,5′-dioxo- |
| | 2,3-dihydrospiro[indene-1,2′-[1,4]ozazolidine]-5-yl]ethanediamide |
| 219-1 | 2-{5-[(azetidin-3-yl)amino]-3′,5′-dioxo-2,3-dihydrospiro[indene-1,2′- |
| | [1,4]ozazolidine]-4′-yl}-N-[(1S)-1-cyclopropylethyl]-N-[(4- |
| | fluorophenyl)methyl]acetamide |
| 220-1 | N-benzyl-N-[(1S)-1-cyclopropylethyl]-2-{3′,5′-dioxo-5-[(2-oxopyrrolidin- |
| | 3-yl)amino]-2,3-dihydrospiro[indene-1,2′-[1,4]ozazolidine]-4′- |
| | yl}acetamide |
| 221-1 | 2-{[4′-({benzyl[(1S)-1-cyclopropylethyl]carbamoyl}methyl)-3′,5′-dioxo- |
| | 2,3-dihydrospiro[indene-1,2′-[1,4]ozazolidine]-5-yl]carbamoyl}-2,2- |
| | dimethylacetic acid |
| 222-1 | N-benzyl-N-[(1S)-1-cyclopropylethyl]-2-{5-[(2-hydroxyethyl)amino]- |
| | 3′,5′-dioxo-2,3-dihydrospiro[indene-1,2′-[1,4]ozazolidine]-4′-yl}acetamide |
| 223-1 | (2R)-2-amino-N-[4′-({[(1S)-1-cyclopropylethyl][(4- |
| | fluorophenyl)methyl]carbamoyl}methyl)-3′,5′-dioxo-2,3- |
| | dihydrospiro[indene-1,2′-[1,4]ozazolidine]-5-yl]propanamide |
| 224-1 | {[4′-({benzyl[(1S)-1-cyclopropylethyl]carbamoyl}methyl)-3′,5′-dioxo-2,3- |
| | dihydrospiro[indene-1,2′-[1,4]ozazolidine]-5-yl]carbamoyl}formic acid |
| 225-1 | (2S)-2-amino-N-[4′-({benzyl[(1S)-1-cyclopropylethyl]carbamoyl}methyl)- |
| | 3′,5′-dioxo-2,3-dihydrospiro[indene-1,2′-[1,4]ozazolidine]-5-yl]-3- |
| | hydroxypropanamide |
| 226-1 | N-[(1S)-1-cyclopropylethyl]-2-{3′,5′-dioxo-5-[(piperidin-3-yl)amino]-2,3- |
| | dihydrospiro[indene-1,2′-[1,4]ozazolidine]-4′-yl}-N-[(4- |
| | fluorophenyl)methyl]acetamide |
| 227-1 | N-benzyl-N-[(1S)-1-cyclopropylethyl]-2-{5-[(methylcarbamoyl)amino]- |
| | 3′,5′-dioxo-2,3-dihydro(3,3-²H₂)spiro[indene-1,2′-[1,4]ozazolidine]-4′- |
| | yl}acetamide |
| 228-1 | N-[(4-fluorophenyl)methyl]-2-{5-[(methylcarbamoyl)amino]-3′,5′-dioxo- |
| | 2,3-dihydrospiro[indene-1,2′-[1,4]ozazolidine]-4′-yl}-N-[(2S)-1,1,1- |
| | trifluoropropan-2-yl]acetamide |
| 229-1 | 2-amino-N-[4′-({benzyl[(1R)-1-cyclopropyl-2,2,2- |
| | trifluoroethyl]carbamoyl}methyl)-3′,5′-dioxo-2,3-dihydrospiro[indene- |
| | 1,2′-[1,4]ozazolidine]-5-yl]-2-methylpropanamide |
| 230-1 | 2-{5-[(methylcarbamoyl)amino]-3′,5′-dioxo-2,3-dihydrospiro[indene-1,2′- |
| | [1,4]ozazolidine]-4′-yl}-N-[(2-methylphenyl)methyl]-N-(2- |
| | methylpropyl)acetamide |
| 231-1 | 2-{5-[(4-aminooxolan-3-yl)amino]-3′,5′-dioxo-2,3-dihydrospiro[indene- |
| | 1,2′-[1,4]ozazolidine]-4′-yl}-N-[(1S)-1-cyclopropylethyl]-N-[(4- |
| | fluorophenyl)methyl]acetamide |
| 232-1 | N-[(4-²H)benzyl]-N-[(1S)-1-cyclopropylethyl]-2-{5- |
| | [(methylcarbamoyl)amino]-3′,5′-dioxo-2,3-dihydrospiro[indene-1,2′- |
| | [1,4]ozazolidine]-4′-yl}acetamide |
| 233-1 | N-[4′-({[(1S)-1-cyclopropylethyl][(4- |
| | fluorophenyl)methyl]carbamoyl}methyl)-3′,5′-dioxo-2,3- |
| | dihydrospiro[indene-1,2′-[1,4]ozazolidine]-5-yl]-2-methyl-2- |
| | (methylamino)propanamide |
| 234-1 | (2R)-2-amino-N-[4′-({[(1S)-1-cyclopropylethyl][(4- |
| | fluorophenyl)methyl]carbamoyl}methyl)-3′,5′-dioxo-2,3- |
| | dihydrospiro[indene-1,2′-[1,4]ozazolidine]-5-yl]-3-methylbutanamide |
| 235-1 | N-benzyl-N-[(1S)-1-cyclopropylethyl]-2-[(1S)-5- |
| | [(methylcarbamoyl)amino]-3′,5′-dioxo-2,3-dihydrospiro[indene-1,2′- |
| | [1,4]ozazolidine]-4′-yl]acetamide |
| 235-2 | N-benzyl-N-[(1S)-1-cyclopropylethyl]-2-[(1R)-5- |
| | [(methylcarbamoyl)amino]-3′,5′-dioxo-2,3-dihydrospiro[indene-1,2′- |
| | [1,4]ozazolidine]-4′-yl]acetamide |
| 236-1 | N-[4′-({benzyl[(1S)-1-cyclopropylethyl]carbamoyl}methyl)-3′,5′-dioxo- |
| | 2,3-dihydrospiro[indene-1,2′-[1,4]ozazolidine]-5-yl]-3-methylazetidine-3- |
| | carboxamide |
| 237-1 | N-[4′-({[(1S)-1-cyclopropylethyl][(4- |
| | fluorophenyl)methyl]carbamoyl}methyl)-3′,5′-dioxo-2,3- |
| | dihydrospiro[indene-1,2′-[1,4]ozazolidine]-5-yl]-3-methylazetidine-3- |
| | carboxamide |
| 238-1 | 2-{5-[(2R)-2-amino-2-cyclopropylacetamido]-3′,5′-dioxo-2,3- |
| | dihydrospiro[indene-1,2′-[1,4]ozazolidine]-4′-yl}-N-[(4- |
| | fluorophenyl)methyl]-N-[(2S)-1,1,1-trifluoropropan-2-yl]acetamide |
| 239-1 | N-[4′-({[(4-fluorophenyl)methyl][(2S)-1,1,1-trifluoropropan-2- |
| | yl]carbamoyl}methyl)-3′,5′-dioxo-2,3-dihydrospiro[indene-1,2′- |
| | [1,4]ozazolidine]-5-yl]-3-methylazetidine-3-carboxamide |
| 240-1 | N-[4′-({benzyl[(1S)-1-cyclopropylethyl]carbamoyl}methyl)-3′,5′-dioxo- |
| | 2,3-dihydrospiro[indene-1,2′-[1,4]ozazolidine]-5-yl]-2-(dimethylamino)-2- |
| | methylpropanamide |
| 241-1 | (2R)-2-amino-N-[4′-({benzyl[(1S)-1- |
| | cyclopropylethyl]carbamoyl}methyl)-3′,5′-dioxo-2,3-dihydrospiro[indene- |
| | 1,2′-[1,4]ozazolidine]-5-yl]-3-hydroxypropanamide |
| 242-1 | N-[(2-amino-1,3-thiazol-5-yl)methyl]-N-[(1S)-1-cyclopropylethyl]-2- |
| | [(4S)-2,5-dioxo-2′,3′-dihydrospiro[imidazolidine-4,1′-indene]-1- |
| | yl]acetamide |
| 243-1 | N-[(1S)-1-cyclopropylethyl]-N-{[2-(dimethylamino)-1,3-thiazol-5- |
| | yl]methyl}-2-[(4S)-2,5-dioxo-2′,3′-dihydrospiro[imidazolidine-4,1′- |
| | indene]-1-yl]acetamide |
| 244-1 | N-[(1S)-1-cyclopropylethyl]-N-{[6-(dimethylamino)pyridin-3-yl]methyl}- |
| | 2-[(4S)-2,5-dioxo-2′,3′-dihydrospiro[imidazolidine-4,1′-indene]-1- |
| | yl]acetamide |
| 245-1 | 2-{5-bromo-3′,5′-dioxo-2,3-dihydrospiro[indene-1,2′-[1,4]ozazolidine]-4′- |
| | yl}-N-[(1S)-1-cyclopropylethyl]-N-[(4-fluorophenyl)methyl]acetamide |
| 246-1 | 2-{5-[(3-aminocyclohexyl)amino]-3′,5′-dioxo-2,3-dihydrospiro[indene- |
| | 1,2′-[1,4]ozazolidine]-4′-yl}-N-[(1S)-1-cyclopropylethyl]-N-[(4- |
| | fluorophenyl)methyl]acetamide |
| 247-1 | N-[(2-amino-1,3-thiazol-4-yl)methyl]-N-[(1S)-1-cyclopropylethyl]-2- |
| | [(4S)-2,5-dioxo-2′,3′-dihydrospiro[imidazolidine-4,1′-indene]-1- |
| | yl]acetamide |
| 248-1 | N-[1-(azetidin-3-yl)ethyl]-N-benzyl-2-{5-[(methylcarbamoyl)amino]-3′,5′- |
| | dioxo-2,3-dihydrospiro[indene-1,2′-[1,4]ozazolidine]-4′-yl}acetamide |
| 249-1 | N-[(1S)-1-cyclopropylethyl]-N-[(4-fluorophenyl)methyl]-2-{1,1,2′,5′- |
| | tetraoxo-2H-spiro[1λ⁶-benzothiophene-3,4′-imidazolidine]-1′- |
| | yl}acetamide |
| 250-1 | N-[(1S)-1-cyclopropylethyl]-2-[(4S)-2,5-dioxo-2′,3′- |
| | dihydrospiro[imidazolidine-4,1′-indene]-1-yl]-N-[(6-hydroxypyridin-3- |
| | yl)methyl]acetamide |
| 251-1 | N-[(1S)-1-cyclopropylethyl]-2-[(4S)-2,5-dioxo-2′,3′- |
| | dihydrospiro[imidazolidine-4,1′-indene]-1-yl]-N-[(6-methoxypyridin-3- |
| | yl)methyl]acetamide |
| 252-1 | 2-amino-N-[4′-({[(4-fluorophenyl)methyl][(2S)-1,1,1-trifluoropropan-2- |
| | yl]carbamoyl}methyl)-3′,5′-dioxo-2,3-dihydrospiro[indene-1,2′- |
| | [1,4]ozazolidine]-5-yl]-2-methylpropanamide |
| 253-1 | N-[(4-fluorophenyl)methyl]-N-[(2S)-1-methoxypropan-2-yl]-2-{5- |
| | [(methylcarbamoyl)amino]-3′,5′-dioxo-2,3-dihydrospiro[indene-1,2′- |
| | [1,4]ozazolidine]-4′-yl}acetamide |
| 254-1 | 3-amino-N-[4′-({benzyl[(1S)-1-cyclopropylethyl]carbamoyl}methyl)-3′,5′- |
| | dioxo-2,3-dihydrospiro[indene-1,2′-[1,4]ozazolidine]-5-yl]oxetane-3- |
| | carboxamide |
| 255-1 | N-[(1S)-1-cyclopropylethyl]-N-{[2-(dimethylamino)-1,3-thiazol-4- |
| | yl]methyl}-2-[(4S)-2,5-dioxo-2′,3′-dihydrospiro[imidazolidine-4,1′- |
| | indene]-1-yl]acetamide |
| 256-1 | N-benzyl-N-[(1R)-1-cyclopropyl-2,2,2-trifluoroethyl]-2-{1,1,2′,5′- |
| | tetraoxo-2H-spiro[1λ⁶-benzothiophene-3,4′-imidazolidine]-1′- |
| | yl}acetamide |
| 257-1 | N-[(6-amino-5-methoxypyridin-3-yl)methyl]-N-[(1S)-1-cyclopropylethyl]- |
| | 2-[(4S)-2,5-dioxo-2′,3′-dihydrospiro[imidazolidine-4,1′-indene]-1- |
| | yl]acetamide |
| 258-1 | N-[(6-aminopyridin-3-yl)methyl]-N-[(1S)-1-cyclopropylethyl]-2-[(4S)- |
| | 2,5-dioxo-2′,3′-dihydrospiro[imidazolidine-4,1′-indene]-1-yl]acetamide |
| 259-1 | N-benzyl-2-{6-bromo-1,1,2′,5′-tetraoxo-2H-spiro[1λ⁶-benzothiophene- |
| | 3,4′-imidazolidine]-1′-yl}-N-[(1S)-1-cyclopropylethyl]acetamide |
| 260-1 | N-benzyl-N-[(1S)-1-cyclopropylethyl]-2-[1,1,2′,5′-tetraoxo-6-(1H-pyrazol- |
| | 3-yl)-2H-spiro[1λ⁶-benzothiophene-3,4′-imidazolidine]-1′-yl]acetamide |
| 261-1 | 2-amino-N-[(1S)-4′-({[(4-bromophenyl)methyl][(1S)-1- |
| | cyclopropylethyl]carbamoyl}methyl)-3′,5′-dioxo-2,3-dihydrospiro[indene- |
| | 1,2′-[1,4]ozazolidine]-5-yl]-2-methylpropanamide |
| 261-2 | 2-amino-N-[(1R)-4′-({[(4-bromophenyl)methyl][(1S)-1- |
| | cyclopropylethyl]carbamoyl}methyl)-3′,5′-dioxo-2,3-dihydrospiro[indene- |
| | 1,2′-[1,4]ozazolidine]-5-yl]-2-methylpropanamide |
| 262-1 | 2-{5-[(2R)-2-amino-2-(oxetan-3-yl)acetamido]-3′,5′-dioxo-2,3- |
| | dihydrospiro[indene-1,2′-[1,4]ozazolidine]-4′-yl}-N-benzyl-N-[(1S)-1- |
| | cyclopropylethyl]acetamide |
| 263-1 | 1-amino-N-[4′-({benzyl[(1S)-1-cyclopropylethyl]carbamoyl}methyl)-3′,5′- |
| | dioxo-2,3-dihydrospiro[indene-1,2′-[1,4]ozazolidine]-5-yl]-3,3- |
| | difluorocyclobutane-1-carboxamide |
| 264-1 | 2-amino-N-[4′-({benzyl[(1S)-1-cyclopropylethyl]carbamoyl}methyl)-3′,5′- |
| | dioxo-2,3-dihydrospiro[indene-1,2′-[1,4]ozazolidine]-5-yl]-3,3,3- |
| | trifluoropropanamide |
| 265-1 | (2R)-2-amino-N-[(1S)-4′-({[(1S)-1-cyclopropylethyl][(4- |
| | fluorophenyl)methyl]carbamoyl}methyl)-3′,5′-dioxo-2,3- |
| | dihydrospiro[indene-1,2′-[1,4]ozazolidine]-5-yl]-3-methylbutanamide |
| 265-2 | (2R)-2-amino-N-[(1R)-4′-({[(1S)-1-cyclopropylethyl][(4- |
| | fluorophenyl)methyl]carbamoyl}methyl)-3′,5′-dioxo-2,3- |
| | dihydrospiro[indene-1,2′-[1,4]ozazolidine]-5-yl]-3-methylbutanamide |
| 266-1 | N-[(4-fluorophenyl)methyl]-2-[(1S)-5-[(methylcarbamoyl)amino]-3′,5′- |
| | dioxo-2,3-dihydrospiro[indene-1,2′-[1,4]ozazolidine]-4′-yl]-N-[(2S)-1,1,1- |
| | trifluoropropan-2-yl]acetamide |
| 266-2 | N-[(4-fluorophenyl)methyl]-2-[(1R)-5-[(methylcarbamoyl)amino]-3′,5′- |
| | dioxo-2,3-dihydrospiro[indene-1,2′-[1,4]ozazolidine]-4′-yl]-N-[(2S)-1,1,1- |
| | trifluoropropan-2-yl]acetamide |
| 267-1 | N-[(1S)-1-cyclopropylethyl]-N-[(4-fluorophenyl)methyl]-2-[(1S)-5- |
| | [(methylcarbamoyl)amino]-3′,5′-dioxo-2,3-dihydrospiro[indene-1,2′- |
| | [1,4]ozazolidine]-4′-yl]acetamide |
| 267-2 | N-[(1S)-1-cyclopropylethyl]-N-[(4-fluorophenyl)methyl]-2-[(1R)-5- |
| | [(methylcarbamoyl)amino]-3′,5′-dioxo-2,3-dihydrospiro[indene-1,2′- |
| | [1,4]ozazolidine]-4′-yl]acetamide |
| 268-1 | (2S)-2-amino-N-[(1S)-4′-({benzyl[(1S)-1- |
| | cyclopropylethyl]carbamoyl}methyl)-3′,5′-dioxo-2,3-dihydrospiro[indene- |
| | 1,2′-[1,4]ozazolidine]-5-yl]-3,3,3-trifluoropropanamide |
| 268-2 | (2R)-2-amino-N-[(1S)-4′-({benzyl[(1S)-1- |
| | cyclopropylethyl]carbamoyl}methyl)-3′,5′-dioxo-2,3-dihydrospiro[indene- |
| | 1,2′-[1,4]ozazolidine]-5-yl]-3,3,3-trifluoropropanamide |
| 268-3 | (2S)-2-amino-N-[(1R)-4′-({benzyl[(1S)-1- |
| | cyclopropylethyl]carbamoyl}methyl)-3′,5′-dioxo-2,3-dihydrospiro[indene- |
| | 1,2′-[1,4]ozazolidine]-5-yl]-3,3,3-trifluoropropanamide |
| 268-4 | (2R)-2-amino-N-[(1R)-4′-({benzyl[(1S)-1- |
| | cyclopropylethyl]carbamoyl}methyl)-3′,5′-dioxo-2,3-dihydrospiro[indene- |
| | 1,2′-[1,4]ozazolidine]-5-yl]-3,3,3-trifluoropropanamide |
| 269-1 | N-benzyl-N-[(1R)-1-cyclopropyl-2,2,2-trifluoroethyl]-2-[(1S)-5- |
| | [(methylcarbamoyl)amino]-3′,5′-dioxo-2,3-dihydrospiro[indene-1,2′- |
| | [1,4]ozazolidine]-4′-yl]acetamide |
| 269-2 | N-benzyl-N-[(1R)-1-cyclopropyl-2,2,2-trifluoroethyl]-2-[(1R)-5- |
| | [(methylcarbamoyl)amino]-3′,5′-dioxo-2,3-dihydrospiro[indene-1,2′- |
| | [1,4]ozazolidine]-4′-yl]acetamide |
| 270-1 | (2R)-2-amino-N-[(1S)-4′-({[(4-fluorophenyl)methyl][(2S)-1,1,1- |
| | trifluoropropan-2-yl]carbamoyl}methyl)-3′,5′-dioxo-2,3- |
| | dihydrospiro[indene-1,2′-[1,4]ozazolidine]-5-yl]-3-methylbutanamide |
| 270-2 | (2R)-2-amino-N-[(1R)-4′-({[(4-fluorophenyl)methyl][(2S)-1,1,1- |
| | trifluoropropan-2-yl]carbamoyl}methyl)-3′,5′-dioxo-2,3- |
| | dihydrospiro[indene-1,2′-[1,4]ozazolidine]-5-yl]-3-methylbutanamide |
| 271-1 | (2R)-2-amino-N-[(1S)-4′-({[(1R)-1-cyclopropyl-2,2,2-trifluoroethyl][(4- |
| | fluorophenyl)methyl]carbamoyl}methyl)-3′,5′-dioxo-2,3- |
| | dihydrospiro[indene-1,2′-[1,4]ozazolidine]-5-yl]-3-methylbutanamide |
| 271-2 | (2R)-2-amino-N-[(1R)-4′-({[(1R)-1-cyclopropyl-2,2,2-trifluoroethyl][(4- |
| | fluorophenyl)methyl]carbamoyl}methyl)-3′,5′-dioxo-2,3- |
| | dihydrospiro[indene-1,2′-[1,4]ozazolidine]-5-yl]-3-methylbutanamide |
| 272-1 | N-[(1R)-1-cyclopropyl-2,2,2-trifluoroethyl]-N-[(4-fluorophenyl)methyl]- |
| | 2-[(1S)-5-[(methylcarbamoyl)amino]-3′,5′-dioxo-2,3-dihydrospiro[indene- |
| | 1,2′-[1,4]ozazolidine]-4′-yl]acetamide |
| 272-2 | N-[(1R)-1-cyclopropyl-2,2,2-trifluoroethyl]-N-[(4-fluorophenyl)methyl]- |
| | 2-[(1R)-5-[(methylcarbamoyl)amino]-3′,5′-dioxo-2,3-dihydrospiro[indene- |
| | 1,2′-[1,4]ozazolidine]-4′-yl]acetamide |
| 273-1 | N-[(1S)-4′-({[(1R)-1-cyclopropyl-2,2,2-trifluoroethyl][(4- |
| | fluorophenyl)methyl]carbamoyl}methyl)-3′,5′-dioxo-2,3- |
| | dihydrospiro[indene-1,2′-[1,4]ozazolidine]-5-yl]azetidine-3-carboxamide |
| 273-2 | N-[(1R)-4′-({[(1R)-1-cyclopropyl-2,2,2-trifluoroethyl][(4- |
| | fluorophenyl)methyl]carbamoyl}methyl)-3′,5′-dioxo-2,3- |
| | dihydrospiro[indene-1,2′-[1,4]ozazolidine]-5-yl]azetidine-3-carboxamide |
| 274-1 | (2R)-2-amino-N-[4′-({[(1R)-1-cyclopropyl-2,2,2-trifluoroethyl][(4- |
| | fluorophenyl)methyl]carbamoyl}methyl)-3′,5′-dioxo-2,3- |
| | dihydrospiro[indene-1,2′-[1,4]ozazolidine]-5-yl]-3-methylbutanamide |
| 275-1 | N-[(1S)-4′-({[(1S)-1-cyclopropylethyl][(4- |
| | fluorophenyl)methyl]carbamoyl}methyl)-3′,5′-dioxo-2,3- |
| | dihydrospiro[indene-1,2′-[1,4]ozazolidine]-5-yl]azetidine-3-carboxamide |
| 275-2 | N-[(1R)-4′-({[(1S)-1-cyclopropylethyl][(4- |
| | fluorophenyl)methyl]carbamoyl}methyl)-3′,5′-dioxo-2,3- |
| | dihydrospiro[indene-1,2′-[1,4]ozazolidine]-5-yl]azetidine-3-carboxamide |
| 276-1 | (2S)-2-amino-N-[4′-({[(1R)-1-cyclopropyl-2,2,2-trifluoroethyl][(4- |
| | fluorophenyl)methyl]carbamoyl}methyl)-3′,5′-dioxo-2,3- |
| | dihydrospiro[indene-1,2′-[1,4]ozazolidine]-5-yl]-3-methylbutanamide |
| 277-1 | (2S)-2-amino-N-[4′-({[(4-fluorophenyl)methyl][(2S)-1,1,1- |
| | trifluoropropan-2-yl]carbamoyl}methyl)-3′,5′-dioxo-2,3- |
| | dihydrospiro[indene-1,2′-[1,4]ozazolidine]-5-yl]-3-methylbutanamide |
| 278-1 | (2R)-2-amino-N-[4′-({[(4-fluorophenyl)methyl][(2S)-1,1,1- |
| | trifluoropropan-2-yl]carbamoyl}methyl)-3′,5′-dioxo-2,3- |
| | dihydrospiro[indene-1,2′-[1,4]ozazolidine]-5-yl]-3-methylbutanamide |
| 279-1 | N-[4′-({[(4-fluorophenyl)methyl][(2S)-1,1,1-trifluoropropan-2- |
| | yl]carbamoyl}methyl)-3′,5′-dioxo-2,3-dihydrospiro[indene-1,2′- |
| | [1,4]ozazolidine]-5-yl]azetidine-3-carboxamide |
| 280-1 | N-benzyl-N-[1-(1-methylazetidin-3-yl)ethyl]-2-{5- |
| | [(methylcarbamoyl)amino]-3′,5′-dioxo-2,3-dihydrospiro[indene-1,2′- |
| | [1,4]ozazolidine]-4′-yl}acetamide |
| 281 | 2-[(1R)-5-bromo-2′,4′-dioxo-2,3-dihydro-3′H-spiro[indene-1,5′- |
| | [1,3]ozazolidin]-3′-yl]-N-(4-fluorobenzyl)-N-[(2S)-1,1,1-trifluoropropan- |
| | 2-yl]acetamide |
| 282 | N-[(1R)-3′-(2-{[(1R)-1-cyclopropylethyl](4-fluorobenzyl)amino}-2- |
| | oxoethyl)-2′,4′-dioxo-2,3-dihydrospiro[indene-1,5′-[1,3]ozazolidin]-5-yl]- |
| | 3,3-difluoroazetidine-1-carboxamide |
| 283 | N-benzyl-2-[(1R)-5-bromo-2′,4′-dioxo-2,3-dihydro-3′H-spiro[indene-1,5′- |
| | [1,3]ozazolidin]-3′-yl]-N-[(1S)-1-cyclopropylethyl]acetamide |
| 284 | 2-[(1S)-5-amino-2′,4′-dioxo-2,3-dihydro-3′H-spiro[indene-1,5′- |
| | [1,3]ozazolidin]-3′-yl]-N-[(1S)-1-cyclopropylethyl]-N-(4- |
| | fluorobenzyl)acetamide |
| 285 | 2-[(1R)-5-amino-2′,4′-dioxo-2,3-dihydro-3′H-spiro[indene-1,5′- |
| | [1,3]ozazolidin]-3′-yl]-N-[(1R)-1-cyclopropylethyl]-N-(4- |
| | fluorobenzyl)acetamide |
| 286 | 2-[(1R)-5-amino-2′,4′-dioxo-2,3-dihydro-3′H-spiro[indene-1,5′- |
| | [1,3]ozazolidin]-3′-yl]-N-benzyl-N-[(1S)-1-cyclopropylethyl]acetamide |
| 287 | N-[(1S)-3′-(2-{[(1R)-1-cyclopropylethyl](4-fluorobenzyl)amino}-2- |
| | oxoethyl)-2′,4′-dioxo-2,3-dihydrospiro[indene-1,5′-[1,3]ozazolidin]-5-yl]- |
| | 3,3-difluoroazetidine-1-carboxamide |
| 288 | N-[(1R)-1-cyclopropylethyl]-N-(4-fluorobenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 289 | 2-(5′-bromo-2,5-dioxo-1′,3′-dihydro-1H-spiro[imidazolidine-4,2′-inden]-1- |
| | yl)-N-[(1S)-1-cyclopropylethyl]-N-(4-fluorobenzyl)acetamide |
| 290 | 2-(6-bromo-1,1-dioxido-2′,5′-dioxo-1′H-spiro[1-benzothiophene-3,4′- |
| | imidazolidin]-1′-yl)-N-[(1S)-1-cyclopropylethyl]-N-(4- |
| | fluorobenzyl)acetamide |
| 291 | N-[(1R)-1-cyclopropylethyl]-N-(4-fluorobenzyl)-2-{(1S)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 292 | N-[(1R)-3′-(2-{[(1S)-1-cyclopropylethyl](4-fluorobenzyl)amino}-2- |
| | oxoethyl)-2′,4′-dioxo-2,3-dihydrospiro[indene-1,5′-[1,3]ozazolidin]-5-yl]- |
| | 3,3-difluoroazetidine-1-carboxamide |
| 293 | N-[(1S)-3′-(2-{[(1S)-1-cyclopropylethyl](4-fluorobenzyl)amino}-2- |
| | oxoethyl)-2′,4′-dioxo-2,3-dihydrospiro[indene-1,5′-[1,3]ozazolidin]-5-yl]- |
| | 3,3-difluoroazetidine-1-carboxamide |
| 294 | tert-butyl 3-[(1R)-3′-(2-{benzyl[(1S)-1-cyclopropylethyl]amino}-2- |
| | oxoethyl)-2′,4′-dioxo-2,3-dihydrospiro[indene-1,5′-[1,3]ozazolidin]-5- |
| | yl]azetidine-1-carboxylate |
| 295 | N-[(1S)-1-cyclopropylethyl]-2-[2,5-dioxo-5′-(6-oxo-1,6-dihydropyridin-3- |
| | yl)-1′,3′-dihydro-1H-spiro[imidazolidine-4,2′-inden]-1-yl]-N-(4- |
| | fluorobenzyl)acetamide |
| 296 | 2-(5′-amino-2,5-dioxo-1′,3′-dihydro-1H-spiro[imidazolidine-4,2′-inden]-1- |
| | yl)-N-[(1S)-1-cyclopropylethyl]-N-(4-fluorobenzyl)acetamide |
| 297 | 2-[(1R)-5-(6-aminopyridin-3-yl)-2′,4′-dioxo-2,3-dihydro-3′H-spiro[indene- |
| | 1,5′-[1,3]ozazolidin]-3′-yl]-N-[(1S)-1-cyclopropylethyl]-N-(4- |
| | fluorobenzyl)acetamide |
| 298 | 2-{(1R)-5-[1-(2-amino-2-oxoethyl)-1H-pyrazol-4-yl]-2′,4′-dioxo-2,3- |
| | dihydro-3′H-spiro[indene-1,5′-[1,3]ozazolidin]-3′-yl}-N-[(1S)-1- |
| | cyclopropylethyl]-N-(4-fluorobenzyl)acetamide |
| 299 | N-[(1S)-1-cyclopropylethyl]-2-[(1R)-2′,4′-dioxo-5-(pyridin-3-yl)-2,3- |
| | dihydro-3′H-spiro[indene-1,5′-[1,3]ozazolidin]-3′-yl]-N-(4- |
| | fluorobenzyl)acetamide |
| 300 | N-[(1S)-1-cyclopropylethyl]-2-[1,1-dioxido-2′,5′-dioxo-6-(6-oxo-1,6- |
| | dihydropyridin-3-yl)-1′H-spiro[1-benzothiophene-3,4′-imidazolidin]-1′- |
| | yl]-N-(4-fluorobenzyl)acetamide |
| 301 | N-benzyl-2-(7-bromo-2′,4′-dioxo-2,3-dihydro-3′H-spiro[chromene-4,5′- |
| | [1,3]ozazolidin]-3′-yl)-N-[(1S)-1-cyclopropylethyl]acetamide |
| 302 | 2-[(1R)-5-(azetidin-3-yl)-2′,4′-dioxo-2,3-dihydro-3′H-spiro[indene-1,5′- |
| | [1,3]ozazolidin]-3′-yl]-N-benzyl-N-[(1S)-1-cyclopropylethyl]acetamide |
| 303 | 2-[(1R)-5-(1-acetylazetidin-3-yl)-2′,4′-dioxo-2,3-dihydro-3′H- |
| | spiro[indene-1,5′-[1,3]ozazolidin]-3′-yl]-N-benzyl-N-[(1S)-1- |
| | cyclopropylethyl]acetamide |
| 304 | N-benzyl-N-[(1S)-1-cyclopropylethyl]-2-[(1R)-5-(1-methylazetidin-3-yl)- |
| | 2′,4′-dioxo-2,3-dihydro-3′H-spiro[indene-1,5′-[1,3]ozazolidin]-3′- |
| | yl]acetamide |
| 305 | 3-[(1R)-3′-(2-{benzyl[(1S)-1-cyclopropylethyl]amino}-2-oxoethyl)-2′,4′- |
| | dioxo-2,3-dihydrospiro[indene-1,5′-[1,3]ozazolidin]-5-yl]-N- |
| | methylazetidine-1-carboxamide |
| 306 | N-benzyl-2-{(1R)-5-[1-(cyclopropylcarbonyl)azetidin-3-yl]-2′,4′-dioxo- |
| | 2,3-dihydro-3′H-spiro[indene-1,5′-[1,3]ozazolidin]-3′-yl}-N-[(1S)-1- |
| | cyclopropylethyl]acetamide |
| 307 | N-benzyl-N-[(1S)-1-cyclopropylethyl]-2-{(1R)-5-[1-(2-hydroxy-2- |
| | methylpropanoyl)azetidin-3-yl]-2′,4′-dioxo-2,3-dihydro-3′H-spiro[indene- |
| | 1,5′-[1,3]ozazolidin]-3′-yl}acetamide |
| 308 | N-benzyl-N-[(1S)-1-cyclopropylethyl]-2-{(1R)-5-[1- |
| | (methylsulfonyl)azetidin-3-yl]-2′,4′-dioxo-2,3-dihydro-3′H-spiro[indene- |
| | 1,5′-[1,3]ozazolidin]-3′-yl}acetamide |
| 309 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]cyclobutanecarboxamide |
| 310 | 3-ethoxy-N-[(1R)-3′-(2-{(4-fluorobenzyl)[(2S)-1,1,1-trifluoropropan-2- |
| | yl]amino}-2-oxoethyl)-2′,4′-dioxo-2,3-dihydrospiro[indene-1,5′- |
| | [1,3]ozazolidin]-5-yl]propanamide |
| 311 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5-yl]- |
| | 5-oxo-D-prolinamide |
| 312 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5-yl]- |
| | 5-oxo-L-prolinamide |
| 313 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]propanamide |
| 314 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]cyclopropanecarboxamide |
| 315 | N-(4-fluorobenzyl)-2-{(1R)-5-[(methoxyacetyl)amino]-2′,4′-dioxo-2,3- |
| | dihydro-3′H-spiro[indene-1,5′-[1,3]ozazolidin]-3′-yl}-N-[(2S)-1,1,1- |
| | trifluoropropan-2-yl]acetamide |
| 316 | 2-{(1R)-5-[(ethoxyacetyl)amino]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 317 | 3,3,3-trifluoro-N-[(1R)-3′-(2-{(4-fluorobenzyl)[(2S)-1,1,1-trifluoropropan- |
| | 2-yl]amino}-2-oxoethyl)-2′,4′-dioxo-2,3-dihydrospiro[indene-1,5′- |
| | [1,3]ozazolidin]-5-yl]propanamide |
| 318 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5-yl]- |
| | 3-methylbutanamide |
| 319 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5-yl]- |
| | 2-methylpropanamide |
| 320 | 2-[(1R)-5-(acetylamino)-2′,4′-dioxo-2,3-dihydro-3′H-spiro[indene-1,5′- |
| | [1,3]ozazolidin]-3′-yl]-N-(4-fluorobenzyl)-N-[(2S)-1,1,1-trifluoropropan- |
| | 2-yl]acetamide |
| 321 | 2-(7-amino-2′,4′-dioxo-2,3-dihydro-3′H-spiro[chromene-4,5′- |
| | [1,3]ozazolidin]-3′-yl)-N-benzyl-N-[(1S)-1-cyclopropylethyl]acetamide |
| 322 | 2-(6-amino-1,1-dioxido-2′,5′-dioxo-1′H-spiro[1-benzothiophene-3,4′- |
| | imidazolidin]-1′-yl)-N-[(1S)-1-cyclopropylethyl]-N-(4- |
| | fluorobenzyl)acetamide |
| 323 | N-[(1S)-1-cyclopropylethyl]-N-(4-fluorobenzyl)-2-{5′- |
| | [(methylcarbamoyl)amino]-2,5-dioxo-1′,3′-dihydro-1H- |
| | spiro[imidazolidine-4,2′-inden]-1-yl}acetamide |
| 324 | N-(4-fluorobenzyl)-2-[(1R)-5-{[(2- |
| | methoxyethyl)(methyl)carbamoyl]amino}-2′,4′-dioxo-2,3-dihydro-3′H- |
| | spiro[indene-1,5′-[1,3]ozazolidin]-3′-yl]-N-[(2S)-1,1,1-trifluoropropan-2- |
| | yl]acetamide |
| 325 | 2-{(1R)-5-[(dimethylcarbamoyl)amino]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 326 | N-(4-fluorobenzyl)-2-[(1R)-5-{[methyl(2- |
| | methylpropyl)carbamoyl]amino}-2′,4′-dioxo-2,3-dihydro-3′H- |
| | spiro[indene-1,5′-[1,3]ozazolidin]-3′-yl]-N-[(2S)-1,1,1-trifluoropropan-2- |
| | yl]acetamide |
| 327 | 2-[(1R)-5-{[(cyclopropylmethyl)carbamoyl]amino}-2′,4′-dioxo-2,3- |
| | dihydro-3′H-spiro[indene-1,5′-[1,3]ozazolidin]-3′-yl]-N-(4-fluorobenzyl)- |
| | N-[(2S)-1,1,1-trifluoropropan-2-yl]acetamide |
| 328 | 2-{(1R)-5-[(cyclobutylcarbamoyl)amino]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 329 | N-(4-fluorobenzyl)-2-[(1R)-5-({[(2R)-1-hydroxy-3-methylbutan-2- |
| | yl]carbamoyl}amino)-2′,4′-dioxo-2,3-dihydro-3′H-spiro[indene-1,5′- |
| | [1,3]ozazolidin]-3′-yl]-N-[(2S)-1,1,1-trifluoropropan-2-yl]acetamide |
| 330 | 2-[(1R)-2′,4′-dioxo-5-({[(2S)-tetrahydrofuran-2- |
| | ylmethyl]carbamoyl}amino)-2,3-dihydro-3′H-spiro[indene-1,5′- |
| | [1,3]ozazolidin]-3′-yl]-N-(4-fluorobenzyl)-N-[(2S)-1,1,1-trifluoropropan- |
| | 2-yl]acetamide |
| 331 | 2-[(1R)-2′,4′-dioxo-5-({[(2R)-tetrahydrofuran-2- |
| | ylmethyl]carbamoyl}amino)-2,3-dihydro-3′H-spiro[indene-1,5′- |
| | [1,3]ozazolidin]-3′-yl]-N-(4-fluorobenzyl)-N-[(2S)-1,1,1-trifluoropropan- |
| | 2-yl]acetamide |
| 332 | 2-[(1R)-2′,4′-dioxo-5-({[2-(propan-2-yloxy)ethyl]carbamoyl}amino)-2,3- |
| | dihydro-3′H-spiro[indene-1,5′-[1,3]ozazolidin]-3′-yl]-N-(4-fluorobenzyl)- |
| | N-[(2S)-1,1,1-trifluoropropan-2-yl]acetamide |
| 333 | 2-{(1R)-5-[(cyclopropylcarbamoyl)amino]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 334 | 2-[(1R)-5-{[ethyl(methyl)carbamoyl]amino}-2′,4′-dioxo-2,3-dihydro-3′H- |
| | spiro[indene-1,5′-[1,3]ozazolidin]-3′-yl]-N-(4-fluorobenzyl)-N-[(2S)-1,1,1- |
| | trifluoropropan-2-yl]acetamide |
| 335 | 2-{(1R)-5-[(diethylcarbamoyl)amino]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 336 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]pyrrolidine-1-carboxamide |
| 337 | 2-[(1R)-5-({[2-(dimethylamino)ethyl](methyl)carbamoyl}amino)-2′,4′- |
| | dioxo-2,3-dihydro-3′H-spiro[indene-1,5′-[1,3]ozazolidin]-3′-yl]-N-(4- |
| | fluorobenzyl)-N-[(2S)-1,1,1-trifluoropropan-2-yl]acetamide |
| 338 | N²-{[(1R)-3′-(2-{(4-fluorobenzyl)[(2S)-1,1,1-trifluoropropan-2-yl]amino}- |
| | 2-oxoethyl)-2′,4′-dioxo-2,3-dihydrospiro[indene-1,5′-[1,3]ozazolidin]-5- |
| | yl]carbamoyl}-L-leucinamide |
| 339 | N-(4-fluorobenzyl)-2-[(1R)-5-{[(2- |
| | hydroxyethyl)(propyl)carbamoyl]amino}-2′,4′-dioxo-2,3-dihydro-3′H- |
| | spiro[indene-1,5′-[1,3]ozazolidin]-3′-yl]-N-[(2S)-1,1,1-trifluoropropan-2- |
| | yl]acetamide |
| 340 | N-(4-fluorobenzyl)-2-[(1R)-5-{[(2-methoxyethyl)carbamoyl]amino}-2′,4′- |
| | dioxo-2,3-dihydro-3′H-spiro[indene-1,5′-[1,3]ozazolidin]-3′-yl]-N-[(2S)- |
| | 1,1,1-trifluoropropan-2-yl]acetamide |
| 341 | N-(4-fluorobenzyl)-2-[(1R)-5-{[(3-hydroxypropyl)carbamoyl]amino}- |
| | 2′,4′-dioxo-2,3-dihydro-3′H-spiro[indene-1,5′-[1,3]ozazolidin]-3′-yl]-N- |
| | [(2S)-1,1,1-trifluoropropan-2-yl]acetamide |
| 342 | N-(4-fluorobenzyl)-2-[(1R)-5-({[(2S)-1-hydroxy-3-methylbutan-2- |
| | yl]carbamoyl}amino)-2′,4′-dioxo-2,3-dihydro-3′H-spiro[indene-1,5′- |
| | [1,3]ozazolidin]-3′-yl]-N-[(2S)-1,1,1-trifluoropropan-2-yl]acetamide |
| 343 | N-(4-fluorobenzyl)-2-[(1R)-5-{[methyl(propan-2-yl)carbamoyl]amino}- |
| | 2′,4′-dioxo-2,3-dihydro-3′H-spiro[indene-1,5′-[1,3]ozazolidin]-3′-yl]-N- |
| | [(2S)-1,1,1-trifluoropropan-2-yl]acetamide |
| 344 | 2-[(1R)-5-{[(2-cyanoethyl)carbamoyl]amino}-2′,4′-dioxo-2,3-dihydro-3′H- |
| | spiro[indene-1,5′-[1,3]ozazolidin]-3′-yl]-N-(4-fluorobenzyl)-N-[(2S)-1,1,1- |
| | trifluoropropan-2-yl]acetamide |
| 345 | N-(4-fluorobenzyl)-2-[(1R)-5-{[methyl(propyl)carbamoyl]amino}-2′,4′- |
| | dioxo-2,3-dihydro-3′H-spiro[indene-1,5′-[1,3]ozazolidin]-3′-yl]-N-[(2S)- |
| | 1,1,1-trifluoropropan-2-yl]acetamide |
| 346 | 2-[5′-(acetylamino)-2,5-dioxo-1′,3′-dihydro-1H-spiro[imidazolidine-4,2′- |
| | inden]-1-yl]-N-[(1S)-1-cyclopropylethyl]-N-(4-fluorobenzyl)acetamide |
| 347 | N-[(1S)-1-cyclopropylethyl]-N-(4-fluorobenzyl)-2-{6- |
| | [(methylcarbamoyl)amino]-1,1-dioxido-2′,5′-dioxo-1′H-spiro[1- |
| | benzothiophene-3,4′-imidazolidin]-1′-yl}acetamide |
| 348 | N-(4-fluorobenzyl)-2-(1′-methyl-2,5-dioxo-2′,3′-dihydro-1H,1′H- |
| | spiro[imidazolidine-4,4′-quinolin]-1-yl)-N-[(2S)-1,1,1-trifluoropropan-2- |
| | yl]acetamide |
| 349 | N-benzyl-N-[(1S)-1-cyclopropylethyl]-2-{7-[(methylcarbamoyl)amino]- |
| | 2′,4′-dioxo-2,3-dihydro-3′H-spiro[chromene-4,5′-[1,3]ozazolidin]-3′- |
| | yl}acetamide |
| 350 | 2-[7-(acetylamino)-2′,4′-dioxo-2,3-dihydro-3′H-spiro[chromene-4,5′- |
| | [1,3]ozazolidin]-3′-yl]-N-benzyl-N-[(1S)-1-cyclopropylethyl]acetamide |
| 351 | N-[(1S)-1-cyclopropylethyl]-N-(4-fluorobenzyl)-2-(3′-hydroxy-2,5-dioxo- |
| | 2′,3′-dihydro-1H-spiro[imidazolidine-4,1′-inden]-1-yl)acetamide |
| 352 | N-[(1S)-1-cyclopropylethyl]-2-[(1R)-2′,4′-dioxo-5-(pyridin-4-yl)-2,3- |
| | dihydro-3′H-spiro[indene-1,5′-[1,3]ozazolidin]-3′-yl]-N-(4- |
| | fluorobenzyl)acetamide |
| 353 | N-[(1S)-1-cyclopropylethyl]-N-(4-fluorobenzyl)-2-[(1R)-5-(6- |
| | hydroxypyridazin-3-yl)-2′,4′-dioxo-2,3-dihydro-3′H-spiro[indene-1,5′- |
| | [1,3]ozazolidin]-3′-yl]acetamide |
| 354 | N-[(1S)-1-cyclopropylethyl]-2-[(1R)-2′,4′-dioxo-5-(1H-pyrazol-4-yl)-2,3- |
| | dihydro-3′H-spiro[indene-1,5′-[1,3]ozazolidin]-3′-yl]-N-(4- |
| | fluorobenzyl)acetamide |
| 355 | N-(4-fluorobenzyl)-N-[(2S)-1,1,1-trifluoropropan-2-yl]-2-(2,3′,5-trioxo- |
| | 2′,3′-dihydro-1H-spiro[imidazolidine-4,1′-inden]-1-yl)acetamide |
| 356 | N-[(1S)-1-cyclopropylethyl]-N-(4-fluorobenzyl)-2-(1′-methyl-2,5-dioxo- |
| | 2′,3′-dihydro-1H,1′H-spiro[imidazolidine-4,4′-quinolin]-1-yl)acetamide |
| 357 | N-(4-fluorobenzyl)-2-[(3′E)-3′-(hydroxyimino)-2,5-dioxo-2′,3′-dihydro- |
| | 1H-spiro[imidazolidine-4,1′-inden]-1-yl]-N-[(2S)-1,1,1-trifluoropropan-2- |
| | yl]acetamide |
| 358 | N-[(1S)-1-cyclopropylethyl]-N-(4-fluorobenzyl)-2-(2,3′,5-trioxo-2′,3′- |
| | dihydro-1H-spiro[imidazolidine-4,1′-inden]-1-yl)acetamide |
| 359 | N-benzyl-N-[(1S)-1-cyclopropylethyl]-2-[2′,4′-dioxo-7-(6-oxo-1,6- |
| | dihydropyridin-3-yl)-2,3-dihydro-3′H-spiro[chromene-4,5′- |
| | [1,3]ozazolidin]-3′-yl]acetamide |
| 360 | N-(4-fluorobenzyl)-2-(3′-hydroxy-2,5-dioxo-2′,3′-dihydro-1H- |
| | spiro[imidazolidine-4,1′-inden]-1-yl)-N-[(2S)-1,1,1-trifluoropropan-2- |
| | yl]acetamide |
| 361 | 2-{(1R)-2′,4′-dioxo-5-[(tetrahydrofuran-2-ylmethyl)amino]-2,3-dihydro- |
| | 3′H-spiro[indene-1,5′-[1,3]ozazolidin]-3′-yl}-N-(4-fluorobenzyl)-N-[(2S)- |
| | 1,1,1-trifluoropropan-2-yl]acetamide |
| 362 | 2-{(1R)-5-[(cyclopentylmethyl)amino]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 363 | 2-{(1R)-5-[(2,2-dimethylbutyl)amino]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 364 | 2-{(1R)-2′,4′-dioxo-5-[(tetrahydrofuran-3-ylmethyl)amino]-2,3-dihydro- |
| | 3′H-spiro[indene-1,5′-[1,3]ozazolidin]-3′-yl}-N-(4-fluorobenzyl)-N-[(2S)- |
| | 1,1,1-trifluoropropan-2-yl]acetamide |
| 365 | N-(4-fluorobenzyl)-2-{(1R)-5-[(2-methylpropyl)amino]-2′,4′-dioxo-2,3- |
| | dihydro-3′H-spiro[indene-1,5′-[1,3]ozazolidin]-3′-yl}-N-[(2S)-1,1,1- |
| | trifluoropropan-2-yl]acetamide |
| 366 | 2-[6-(acetylamino)-1,1-dioxido-2′,5′-dioxo-1′H-spiro[1-benzothiophene- |
| | 3,4′-imidazolidin]-1′-yl]-N-[(1S)-1-cyclopropylethyl]-N-(4- |
| | fluorobenzyl)acetamide |
| 367 | tert-butyl [(2R)-1-{[3′-(2-{benzyl[(1S)-1-cyclopropylethyl]amino}-2- |
| | oxoethyl)-2′,4′-dioxo-2,3-dihydrospiro[chromene-4,5′-[1,3]ozazolidin]-7- |
| | yl]amino}-3-methyl-1-oxobutan-2-yl]carbamate |
| 368 | N-[3′-(2-{benzyl[(1S)-1-cyclopropylethyl]amino}-2-oxoethyl)-2′,4′-dioxo- |
| | 2,3-dihydrospiro[chromene-4,5′-[1,3]ozazolidin]-7-yl]-D-valinamide |
| 369 | 2-[(1R)-6-amino-2′,4′-dioxo-2,3-dihydro-3′H-spiro[indene-1,5′- |
| | [1,3]ozazolidin]-3′-yl]-N-benzyl-N-[(1S)-1-cyclopropylethyl]acetamide |
| 370 | 2-[(1R)-6-(acetylamino)-2′,4′-dioxo-2,3-dihydro-3′H-spiro[indene-1,5′- |
| | [1,3]ozazolidin]-3′-yl]-N-benzyl-N-[(1S)-1-cyclopropylethyl]acetamide |
| 371 | N-benzyl-N-[(1S)-1-cyclopropylethyl]-2-{(1R)-6- |
| | [(methylcarbamoyl)amino]-2′,4′-dioxo-2,3-dihydro-3′H-spiro[indene-1,5′- |
| | [1,3]ozazolidin]-3′-yl}acetamide |
| 372 | 2-[(1S)-6-amino-2′,4′-dioxo-2,3-dihydro-3′H-spiro[indene-1,5′- |
| | [1,3]ozazolidin]-3′-yl]-N-benzyl-N-[(1S)-1-cyclopropylethyl]acetamide |
| 373 | 2-[(1S)-6-(acetylamino)-2′,4′-dioxo-2,3-dihydro-3′H-spiro[indene-1,5′- |
| | [1,3]ozazolidin]-3′-yl]-N-benzyl-N-[(1S)-1-cyclopropylethyl]acetamide |
| 374 | N-benzyl-N-[(1S)-1-cyclopropylethyl]-2-{(1S)-6- |
| | [(methylcarbamoyl)amino]-2′,4′-dioxo-2,3-dihydro-3′H-spiro[indene-1,5′- |
| | [1,3]ozazolidin]-3′-yl}acetamide |
| 375 | N-[(1S)-1-cyclopropylethyl]-2-[(1R)-2′,4′-dioxo-5-(pyridin-2-yl)-2,3- |
| | dihydro-3′H-spiro[indene-1,5′-[1,3]ozazolidin]-3′-yl]-N-(4- |
| | fluorobenzyl)acetamide |
| 376 | 2-(7′-bromo-2,5-dioxo-3′,4′-dihydro-1H,1′H-spiro[imidazolidine-4,2′- |
| | naphthalen]-1-yl)-N-[(1S)-1-cyclopropylethyl]-N-(4- |
| | fluorobenzyl)acetamide |
| 377 | N-[(1S)-1-cyclopropylethyl]-N-(3-fluorobenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 378 | N-[(1S)-1-cyclopropylethyl]-N-(3,4-difluorobenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 379 | N-(4-fluorobenzyl)-2-(3′-fluoro-2,5-dioxo-2′,3′-dihydro-1H- |
| | spiro[imidazolidine-4,1′-inden]-1-yl)-N-[(2S)-1,1,1-trifluoropropan-2- |
| | yl]acetamide |
| 380 | 2-(3′-amino-2,5-dioxo-2′,3′-dihydro-1H-spiro[imidazolidine-4,1′-inden]-1- |
| | yl)-N-(4-fluorobenzyl)-N-[(2S)-1,1,1-trifluoropropan-2-yl]acetamide |
| 381 | N-(4-fluorobenzyl)-2-[3′-(methylamino)-2,5-dioxo-2′,3′-dihydro-1H- |
| | spiro[imidazolidine-4,1′-inden]-1-yl]-N-[(2S)-1,1,1-trifluoropropan-2- |
| | yl]acetamide |
| 382 | N-[(1S)-1-cyclopropyl-2,2,2-trifluoroethyl]-N-(4-fluorobenzyl)-2-{7′- |
| | [(methylcarbamoyl)amino]-2,4-dioxo-2′,3′-dihydro-3H-spiro[1,3- |
| | ozazolidine-5,4′-thiochromen]-3-yl}acetamide |
| 383 | 2-[(1R)-5-(carbamoylamino)-2′,4′-dioxo-2,3-dihydro-3′H-spiro[indene- |
| | 1,5′-[1,3]ozazolidin]-3′-yl]-N-(4-fluorobenzyl)-N-[(2S)-1,1,1- |
| | trifluoropropan-2-yl]acetamide |
| 384 | 2-(7′-amino-2,5-dioxo-3′,4′-dihydro-1H,1′H-spiro[imidazolidine-4,2′- |
| | naphthalen]-1-yl)-N-[(1S)-1-cyclopropylethyl]-N-(4- |
| | fluorobenzyl)acetamide |
| 385 | N-[(1S)-1-cyclopropyl-2,2,2-trifluoroethyl]-N-(4-fluorobenzyl)-2-{7′- |
| | [(methylcarbamoyl)amino]-1′,1′-dioxido-2,4-dioxo-2′,3′-dihydro-3H- |
| | spiro[1,3-ozazolidine-5,4′-thiochromen]-3-yl}acetamide |
| 386 | 2-(6-bromo-1,1-dioxido-2′,5′-dioxo-1′H-spiro[1-benzothiophene-3,4′- |
| | imidazolidin]-1′-yl)-N-(4-fluorobenzyl)-N-[(2S)-1,1,1-trifluoropropan-2- |
| | yl]acetamide |
| 387 | N-(4-fluorobenzyl)-2-((R)-5-(3-(N-methylsulfamoyl)ureido)-2′,4′-dioxo- |
| | 2,3-dihydrospiro[indene-1,5′-ozazolidine]-3′-yl)-N-((S)-1,1,1- |
| | trifluoropropan-2-yl)acetamide |
| 388 | methyl {[(1R)-3′-(2-{(4-fluorobenzyl)[(2S)-1,1,1-trifluoropropan-2- |
| | yl]amino}-2-oxoethyl)-2′,4′-dioxo-2,3-dihydrospiro[indene-1,5′- |
| | [1,3]ozazolidin]-5-yl]carbamoyl}sulfamate |
| 389 | 2-((R)-5-(3-(N-(cyclopropylmethyl)sulfamoyl)ureido)-2′,4′-dioxo-2,3- |
| | dihydrospiro[indene-1,5′-ozazolidine]-3′-yl)-N-(4-fluorobenzyl)-N-((S)- |
| | 1,1,1-trifluoropropan-2-yl)acetamide |
| 390 | N-[(1S)-1-cyclopropylethyl]-N-(4-fluorobenzyl)-2-{7′- |
| | [(methylcarbamoyl)amino]-2,5-dioxo-3′,4′-dihydro-1H,1′H- |
| | spiro[imidazolidine-4,2′-naphthalen]-1-yl}acetamide |
| 391 | 2-[7′-(acetylamino)-2,5-dioxo-3′,4′-dihydro-1H,1′H-spiro[imidazolidine- |
| | 4,2′-naphthalen]-1-yl]-N-[(1S)-1-cyclopropylethyl]-N-(4- |
| | fluorobenzyl)acetamide |
| 392 | (1R)-3′-(2-{benzyl[(1S)-1-cyclopropylethyl]amino}-2-oxoethyl)-N-(2- |
| | methylpropyl)-2′,4′-dioxo-2,3-dihydrospiro[indene-1,5′-[1,3]ozazolidine]- |
| | 5-carboxamide |
| 393 | N-[(1S)-1-cyclopropylethyl]-N-(2-fluorobenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 394 | N-[(1S)-1-cyclopropylethyl]-N-(2,4-difluorobenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 395 | N-[(1S)-1-cyclopropylethyl]-N-(2,3-difluorobenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 396 | N-[(1S)-1-cyclopropylethyl]-N-(2,5-difluorobenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 397 | N-(2-chloro-4-fluorobenzyl)-N-[(1S)-1-cyclopropylethyl]-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 398 | N-(3-chloro-4-fluorobenzyl)-N-[(1S)-1-cyclopropylethyl]-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 399 | N-(4-chlorobenzyl)-N-[(1S)-1-cyclopropylethyl]-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 400 | N-[(1S)-1-cyclopropylethyl]-2-{(1R)-5-[(methylcarbamoyl)amino]-2′,4′- |
| | dioxo-2,3-dihydro-3′H-spiro[indene-1,5′-[1,3]ozazolidin]-3′-yl}-N-[4- |
| | (trifluoromethyl)benzyl]acetamide |
| 401 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]benzamide |
| 402 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5-yl]- |
| | 1-methyl-1H-pyrazole-4-carboxamide |
| 403 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5-yl]- |
| | 1-methyl-1H-pyrazole-3-carboxamide |
| 404 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]pyridine-4-carboxamide |
| 405 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]pyridine-2-carboxamide |
| 406 | 2-(5-bromo-4-fluoro-2′,4′-dioxo-2,3-dihydro-3′H-spiro[indene-1,5′- |
| | [1,3]ozazolidin]-3′-yl)-N-[(1S)-1-cyclopropyl-2,2,2-trifluoroethyl]-N-(4- |
| | fluorobenzyl)acetamide |
| 407 | 2-(5-bromo-6-fluoro-2′,4′-dioxo-2,3-dihydro-3′H-spiro[indene-1,5′- |
| | [1,3]ozazolidin]-3′-yl)-N-[(1S)-1-cyclopropyl-2,2,2-trifluoroethyl]-N-(4- |
| | fluorobenzyl)acetamide |
| 408 | (1R)-3′-(2-{benzyl[(1S)-1-cyclopropylethyl]amino}-2-oxoethyl)-N-[(2R)- |
| | 1-hydroxy-3-methylbutan-2-yl]-2′,4′-dioxo-2,3-dihydrospiro[indene-1,5′- |
| | [1,3]ozazolidine]-5-carboxamide |
| 409 | N-[(1S)-1-cyclopropylethyl]-2-{(1R)-5-[(methylcarbamoyl)amino]-2′,4′- |
| | dioxo-2,3-dihydro-3′H-spiro[indene-1,5′-[1,3]ozazolidin]-3′-yl}-N-[(5- |
| | methyl-1,3,4-thiadiazol-2-yl)methyl]acetamide |
| 410 | N-(4-fluorobenzyl)-2-(2′-methyl-2,5-dioxo-2′,3′-dihydro-1H- |
| | spiro[imidazolidine-4,1′-inden]-1-yl)-N-[(2S)-1,1,1-trifluoropropan-2- |
| | yl]acetamide |
| 411 | 2-(5-amino-4-fluoro-2′,4′-dioxo-2,3-dihydro-3′H-spiro[indene-1,5′- |
| | [1,3]ozazolidin]-3′-yl)-N-[(1S)-1-cyclopropyl-2,2,2-trifluoroethyl]-N-(4- |
| | fluorobenzyl)acetamide |
| 412 | N-[(1S)-1-cyclopropyl-2,2,2-trifluoroethyl]-N-(4-fluorobenzyl)-2-{4- |
| | fluoro-5-[(methylcarbamoyl)amino]-2′,4′-dioxo-2,3-dihydro-3′H- |
| | spiro[indene-1,5′-[1,3]ozazolidin]-3′-yl}acetamide |
| 413 | N-(4-cyanobenzyl)-N-[(1S)-1-cyclopropylethyl]-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 414 | N-[(1S)-1-cyclopropylethyl]-N-(3-methylbenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 415 | N-[(1S)-1-cyclopropylethyl]-N-(3,5-difluorobenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 416 | N-(3-chlorobenzyl)-N-[(1S)-1-cyclopropylethyl]-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 417 | N-(2-chlorobenzyl)-N-[(1S)-1-cyclopropylethyl]-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 418 | N-[(1S)-1-cyclopropylethyl]-N-(2,4-dichlorobenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 419 | N-[(1S)-1-cyclopropylethyl]-N-(4-methoxybenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 420 | N-[(1S)-1-cyclopropylethyl]-N-(3-methoxybenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 421 | N-[(1S)-1-cyclopropylethyl]-N-(4-methylbenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 422 | N-(4-fluorobenzyl)-2-[(1R)-5-{[(1-methyl-1H-pyrazol-4- |
| | yl)carbamoyl]amino}-2′,4′-dioxo-2,3-dihydro-3′H-spiro[indene-1,5′- |
| | [1,3]ozazolidin]-3′-yl]-N-[(2S)-1,1,1-trifluoropropan-2-yl]acetamide |
| 423 | N-[1-(2-{[(1S)-1-cyclopropylethyl](4-fluorobenzyl)amino}-2-oxoethyl)- |
| | 2,5-dioxo-3′,4′-dihydro-1′H-spiro[imidazolidine-4,2′-naphthalen]-7′-yl]-5- |
| | oxo-D-prolinamide |
| 424 | 2-{7-[1-(2-amino-2-oxoethyl)-1H-pyrazol-4-yl]-2′,4′-dioxo-2,3-dihydro- |
| | 3′H-spiro[chromene-4,5′-[1,3]ozazolidin]-3′-yl}-N-benzyl-N-[(1S)-1- |
| | cyclopropylethyl]acetamide |
| 425 | 2-{7′-[1-(2-amino-2-oxoethyl)-1H-pyrazol-4-yl]-2,5-dioxo-3′,4′-dihydro- |
| | 1H,1′H-spiro[imidazolidine-4,2′-naphthalen]-1-yl}-N-[(1S)-1- |
| | cyclopropylethyl]-N-(4-fluorobenzyl)acetamide |
| 426 | 2-{(1R)-2′,4′-dioxo-5-[(phenylcarbamoyl)amino]-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 427 | 2-{(1R)-2′,4′-dioxo-5-[(pyridin-3-ylcarbamoyl)amino]-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 428 | 2-(5′-bromo-2,3′,5-trioxo-2′,3′-dihydro-1H-spiro[imidazolidine-4,1′- |
| | inden]-1-yl)-N-(4-fluorobenzyl)-N-[(2S)-1,1,1-trifluoropropan-2- |
| | yl]acetamide |
| 429 | 2-(5-amino-6-fluoro-2′,4′-dioxo-2,3-dihydro-3′H-spiro[indene-1,5′- |
| | [1,3]ozazolidin]-3′-yl)-N-[(1S)-1-cyclopropyl-2,2,2-trifluoroethyl]-N-(4- |
| | fluorobenzyl)acetamide |
| 430 | N-[(1S)-1-cyclopropyl-2,2,2-trifluoroethyl]-N-(4-fluorobenzyl)-2-{6- |
| | fluoro-5-[(methylcarbamoyl)amino]-2′,4′-dioxo-2,3-dihydro-3′H- |
| | spiro[indene-1,5′-[1,3]ozazolidin]-3′-yl}acetamide |
| 431 | N-[(1S)-1-cyclopropylethyl]-N-(4-fluorobenzyl)-2-[(1R)-5-{1-[2- |
| | (methylamino)-2-oxoethyl]-1H-pyrazol-4-yl}-2′,4′-dioxo-2,3-dihydro-3′H- |
| | spiro[indene-1,5′-[1,3]ozazolidin]-3′-yl]acetamide |
| 432 | N-(4-fluorobenzyl)-2-[(1R)-5-{1-[2-(methylamino)-2-oxoethyl]-1H- |
| | pyrazol-4-yl}-2′,4′-dioxo-2,3-dihydro-3′H-spiro[indene-1,5′- |
| | [1,3]ozazolidin]-3′-yl]-N-[(2S)-1,1,1-trifluoropropan-2-yl]acetamide |
| 433 | 2-[(1R)-5-{1-[2-(dimethylamino)-2-oxoethyl]-1H-pyrazol-4-yl}-2′,4′- |
| | dioxo-2,3-dihydro-3′H-spiro[indene-1,5′-[1,3]ozazolidin]-3′-yl]-N-(4- |
| | fluorobenzyl)-N-[(2S)-1,1,1-trifluoropropan-2-yl]acetamide |
| 434 | 2-{(1R)-5-[1-(2-amino-2-oxoethyl)-1H-pyrazol-4-yl]-2′,4′-dioxo-2,3- |
| | dihydro-3′H-spiro[indene-1,5′-[1,3]ozazolidin]-3′-yl}-N-(4-fluorobenzyl)- |
| | N-[(2S)-1,1,1-trifluoropropan-2-yl]acetamide |
| 435 | 2-{(1R)-5-[6-(dimethylamino)pyridin-2-yl]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 436 | N-(4-fluorobenzyl)-2-[(1R)-5-(4-methylpyrimidin-5-yl)-2′,4′-dioxo-2,3- |
| | dihydro-3′H-spiro[indene-1,5′-[1,3]ozazolidin]-3′-yl]-N-[(2S)-1,1,1- |
| | trifluoropropan-2-yl]acetamide |
| 437 | 2-{(1R)-5-[5-(difluoromethyl)pyridin-3-yl]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 438 | N-(4-fluorobenzyl)-2-[(1R)-5-(6-methoxypyridin-3-yl)-2′,4′-dioxo-2,3- |
| | dihydro-3′H-spiro[indene-1,5′-[1,3]ozazolidin]-3′-yl]-N-[(2S)-1,1,1- |
| | trifluoropropan-2-yl]acetamide |
| 439 | N-(4-fluorobenzyl)-2-{(1R)-5-[1-(2-methylpropyl)-1H-pyrazol-4-yl]-2′,4′- |
| | dioxo-2,3-dihydro-3′H-spiro[indene-1,5′-[1,3]ozazolidin]-3′-yl}-N-[(2S)- |
| | 1,1,1-trifluoropropan-2-yl]acetamide |
| 440 | 2-{(1R)-5-[4-(difluoromethyl)pyridin-2-yl]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 441 | N-(4-fluorobenzyl)-2-[(1R)-5-(3-fluoropyridin-2-yl)-2′,4′-dioxo-2,3- |
| | dihydro-3′H-spiro[indene-1,5′-[1,3]ozazolidin]-3′-yl]-N-[(2S)-1,1,1- |
| | trifluoropropan-2-yl]acetamide |
| 442 | 2-[(1R)-2′,4′-dioxo-5-(1,3-thiazol-4-yl)-2,3-dihydro-3′H-spiro[indene-1,5′- |
| | [1,3]ozazolidin]-3′-yl]-N-(4-fluorobenzyl)-N-[(2S)-1,1,1-trifluoropropan- |
| | 2-yl]acetamide |
| 443 | 4-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5-yl]- |
| | N-methylbenzamide |
| 444 | 2-{(1R)-5-[2-(acetylamino)phenyl]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 445 | 2-[(1R)-5-(5-cyanopyridin-3-yl)-2′,4′-dioxo-2,3-dihydro-3′H-spiro[indene- |
| | 1,5′-[1,3]ozazolidin]-3′-yl]-N-(4-fluorobenzyl)-N-[(2S)-1,1,1- |
| | trifluoropropan-2-yl]acetamide |
| 446 | N-(4-fluorobenzyl)-2-[(1R)-5-(1-methyl-1H-1,2,4-triazol-5-yl)-2′,4′-dioxo- |
| | 2,3-dihydro-3′H-spiro[indene-1,5′-[1,3]ozazolidin]-3′-yl]-N-[(2S)-1,1,1- |
| | trifluoropropan-2-yl]acetamide |
| 447 | N-(4-fluorobenzyl)-2-[(1R)-5-(5-methoxypyridin-3-yl)-2′,4′-dioxo-2,3- |
| | dihydro-3′H-spiro[indene-1,5′-[1,3]ozazolidin]-3′-yl]-N-[(2S)-1,1,1- |
| | trifluoropropan-2-yl]acetamide |
| 448 | 2-[(1R)-5-(6-cyanopyridin-2-yl)-2′,4′-dioxo-2,3-dihydro-3′H-spiro[indene- |
| | 1,5′-[1,3]ozazolidin]-3′-yl]-N-(4-fluorobenzyl)-N-[(2S)-1,1,1- |
| | trifluoropropan-2-yl]acetamide |
| 449 | N-(4-fluorobenzyl)-2-[(1R)-5-(5-fluoro-3-methylpyridin-2-yl)-2′,4′-dioxo- |
| | 2,3-dihydro-3′H-spiro[indene-1,5′-[1,3]ozazolidin]-3′-yl]-N-[(2S)-1,1,1- |
| | trifluoropropan-2-yl]acetamide |
| 450 | N-(4-fluorobenzyl)-2-[(1R)-5-(5-methyl-1,3,4-thiadiazol-2-yl)-2′,4′-dioxo- |
| | 2,3-dihydro-3′H-spiro[indene-1,5′-[1,3]ozazolidin]-3′-yl]-N-[(2S)-1,1,1- |
| | trifluoropropan-2-yl]acetamide |
| 451 | N-(4-fluorobenzyl)-2-[(1R)-5-(4-methoxypyridin-3-yl)-2′,4′-dioxo-2,3- |
| | dihydro-3′H-spiro[indene-1,5′-[1,3]ozazolidin]-3′-yl]-N-[(2S)-1,1,1- |
| | trifluoropropan-2-yl]acetamide |
| 452 | N-(4-fluorobenzyl)-2-[(1R)-5-(6-methoxypyridin-2-yl)-2′,4′-dioxo-2,3- |
| | dihydro-3′H-spiro[indene-1,5′-[1,3]ozazolidin]-3′-yl]-N-[(2S)-1,1,1- |
| | trifluoropropan-2-yl]acetamide |
| 453 | 2-{(1R)-5-[2-(dimethylamino)pyrimidin-5-yl]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 454 | 2-{(1R)-5-[6-(acetylamino)pyridin-3-yl]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 455 | N-(4-fluorobenzyl)-2-[(1R)-5-(1-methyl-1H-1,2,4-triazol-3-yl)-2′,4′-dioxo- |
| | 2,3-dihydro-3′H-spiro[indene-1,5′-[1,3]ozazolidin]-3′-yl]-N-[(2S)-1,1,1- |
| | trifluoropropan-2-yl]acetamide |
| 456 | N-(4-fluorobenzyl)-2-[(1R)-5-(1-methyl-1H-pyrazol-4-yl)-2′,4′-dioxo-2,3- |
| | dihydro-3′H-spiro[indene-1,5′-[1,3]ozazolidin]-3′-yl]-N-[(2S)-1,1,1- |
| | trifluoropropan-2-yl]acetamide |
| 457 | N-cyclopropyl-5-[(1R)-3′-(2-{(4-fluorobenzyl)[(2S)-1,1,1-trifluoropropan- |
| | 2-yl]amino}-2-oxoethyl)-2′,4′-dioxo-2,3-dihydrospiro[indene-1,5′- |
| | [1,3]ozazolidin]-5-yl]furan-2-carboxamide |
| 458 | N-(4-fluorobenzyl)-2-[(1R)-5-(5-fluoropyridin-2-yl)-2′,4′-dioxo-2,3- |
| | dihydro-3′H-spiro[indene-1,5′-[1,3]ozazolidin]-3′-yl]-N-[(2S)-1,1,1- |
| | trifluoropropan-2-yl]acetamide |
| 459 | 4-fluoro-3-[(1R)-3′-(2-{(4-fluorobenzyl)[(2S)-1,1,1-trifluoropropan-2- |
| | yl]amino}-2-oxoethyl)-2′,4′-dioxo-2,3-dihydrospiro[indene-1,5′- |
| | [1,3]ozazolidin]-5-yl]benzamide |
| 460 | 5-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5-yl]- |
| | N-methylpyridine-2-carboxamide |
| 461 | 6-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]pyridine-3-carboxamide |
| 462 | N-(4-fluorobenzyl)-2-[(1R)-5-(2-methoxypyrimidin-5-yl)-2′,4′-dioxo-2,3- |
| | dihydro-3′H-spiro[indene-1,5′-[1,3]ozazolidin]-3′-yl]-N-[(2S)-1,1,1- |
| | trifluoropropan-2-yl]acetamide |
| 463 | 6-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]pyridine-2-carboxamide |
| 464 | 4-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]thiophene-3-carboxamide |
| 465 | 2-{(1R)-5-[4-(acetylamino)phenyl]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 466 | 2-[(1R)-5-(1,3-dimethyl-2,4-dioxo-1,2,3,4-tetrahydropyrimidin-5-yl)-2′,4′- |
| | dioxo-2,3-dihydro-3′H-spiro[indene-1,5′-[1,3]ozazolidin]-3′-yl]-N-(4- |
| | fluorobenzyl)-N-[(2S)-1,1,1-trifluoropropan-2-yl]acetamide |
| 467 | 2-[(1R)-5-(4-cyanopyridin-2-yl)-2′,4′-dioxo-2,3-dihydro-3′H-spiro[indene- |
| | 1,5′-[1,3]ozazolidin]-3′-yl]-N-(4-fluorobenzyl)-N-[(2S)-1,1,1- |
| | trifluoropropan-2-yl]acetamide |
| 468 | 2-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]pyridine-4-carboxamide |
| 469 | 2-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]benzamide |
| 470 | 4-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]benzamide |
| 471 | 2-{(1R)-5-[3-(acetylamino)phenyl]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 472 | 2-[(1R)-5-(6-cyano-5-methoxypyridin-3-yl)-2′,4′-dioxo-2,3-dihydro-3′H- |
| | spiro[indene-1,5′-[1,3]ozazolidin]-3′-yl]-N-(4-fluorobenzyl)-N-[(2S)-1,1,1- |
| | trifluoropropan-2-yl]acetamide |
| 473 | N-(4-fluorobenzyl)-2-[(1R)-5-(1-methyl-1H-pyrazol-3-yl)-2′,4′-dioxo-2,3- |
| | dihydro-3′H-spiro[indene-1,5′-[1,3]ozazolidin]-3′-yl]-N-[(2S)-1,1,1- |
| | trifluoropropan-2-yl]acetamide |
| 474 | 2-[(1R)-2′,4′-dioxo-5-(1,3,5-trimethyl-1H-pyrazol-4-yl)-2,3-dihydro-3′H- |
| | spiro[indene-1,5′-[1,3]ozazolidin]-3′-yl]-N-(4-fluorobenzyl)-N-[(2S)-1,1,1- |
| | trifluoropropan-2-yl]acetamide |
| 475 | 2-[(1R)-5-(3,4-difluorophenyl)-2′,4′-dioxo-2,3-dihydro-3′H-spiro[indene- |
| | 1,5′-[1,3]ozazolidin]-3′-yl]-N-(4-fluorobenzyl)-N-[(2S)-1,1,1- |
| | trifluoropropan-2-yl]acetamide |
| 476 | 2-[(1R)-5-(2,5-difluoro-4-methoxyphenyl)-2′,4′-dioxo-2,3-dihydro-3′H- |
| | spiro[indene-1,5′-[1,3]ozazolidin]-3′-yl]-N-(4-fluorobenzyl)-N-[(2S)-1,1,1- |
| | trifluoropropan-2-yl]acetamide |
| 477 | 2-[(1R)-2′,4′-dioxo-5-(pyrimidin-5-yl)-2,3-dihydro-3′H-spiro[indene-1,5′- |
| | [1,3]ozazolidin]-3′-yl]-N-(4-fluorobenzyl)-N-[(2S)-1,1,1-trifluoropropan- |
| | 2-yl]acetamide |
| 478 | 2-{(1R)-2′,4′-dioxo-5-[5-(trifluoromethyl)pyridin-3-yl]-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 479 | 2-[(1R)-2′,4′-dioxo-5-(1,3-thiazol-2-yl)-2,3-dihydro-3′H-spiro[indene-1,5′- |
| | [1,3]ozazolidin]-3′-yl]-N-(4-fluorobenzyl)-N-[(2S)-1,1,1-trifluoropropan- |
| | 2-yl]acetamide |
| 480 | 2-[(1R)-5-(2-cyanopyridin-4-yl)-2′,4′-dioxo-2,3-dihydro-3′H-spiro[indene- |
| | 1,5′-[1,3]ozazolidin]-3′-yl]-N-(4-fluorobenzyl)-N-[(2S)-1,1,1- |
| | trifluoropropan-2-yl]acetamide |
| 481 | 2-[(1R)-2′,4′-dioxo-5-(pyrazin-2-yl)-2,3-dihydro-3′H-spiro[indene-1,5′- |
| | [1,3]ozazolidin]-3′-yl]-N-(4-fluorobenzyl)-N-[(2S)-1,1,1-trifluoropropan- |
| | 2-yl]acetamide |
| 482 | 2-{(1R)-5-[4-(carbamoylamino)phenyl]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 483 | 2-[(1R)-5-(2-cyanopyridin-3-yl)-2′,4′-dioxo-2,3-dihydro-3′H-spiro[indene- |
| | 1,5′-[1,3]ozazolidin]-3′-yl]-N-(4-fluorobenzyl)-N-[(2S)-1,1,1- |
| | trifluoropropan-2-yl]acetamide |
| 484 | 2-[(1R)-5-(6-cyano-5-fluoropyridin-3-yl)-2′,4′-dioxo-2,3-dihydro-3′H- |
| | spiro[indene-1,5′-[1,3]ozazolidin]-3′-yl]-N-(4-fluorobenzyl)-N-[(2S)-1,1,1- |
| | trifluoropropan-2-yl]acetamide |
| 485 | 3-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]benzamide |
| 486 | N-(4-fluorobenzyl)-N-[(2S)-1,1,1-trifluoropropan-2-yl]-2-(2,2′,5-trioxo- |
| | 2′,3′-dihydro-1H,1′H-spiro[imidazolidine-4,4′-quinolin]-1-yl)acetamide |
| 487 | 2-(2,5-dioxo-1H-spiro[imidazolidine-4,1′-inden]-1-yl)-N-(4-fluorobenzyl)- |
| | N-[(2S)-1,1,1-trifluoropropan-2-yl]acetamide |
| 488 | 2-{5′-[1-(2-amino-2-oxoethyl)-1H-pyrazol-4-yl]-2,3′,5-trioxo-2′,3′- |
| | dihydro-1H-spiro[imidazolidine-4,1′-inden]-1-yl}-N-(4-fluorobenzyl)-N- |
| | [(2S)-1,1,1-trifluoropropan-2-yl]acetamide |
| 489 | N-(4-fluorobenzyl)-N-[(2S)-1,1,1-trifluoropropan-2-yl]-2-[2,3′,5-trioxo-5′- |
| | (6-oxo-1,6-dihydropyridin-3-yl)-2′,3′-dihydro-1H-spiro[imidazolidine-4,1′- |
| | inden]-1-yl]acetamide |
| 490 | 2-[(1R)-5-{[(4-aminophenyl)carbamoyl]amino}-2′,4′-dioxo-2,3-dihydro- |
| | 3′H-spiro[indene-1,5′-[1,3]ozazolidin]-3′-yl]-N-(4-fluorobenzyl)-N-[(2S)- |
| | 1,1,1-trifluoropropan-2-yl]acetamide |
| 491 | N-[(1S)-1-cyclopropylethyl]-2-[(4R)-2,5-dioxo-2′,3′-dihydro-1H- |
| | spiro[imidazolidine-4,1′-inden]-1-yl]-N-(4-fluorobenzyl)acetamide |
| 492 | N-[(1S)-1-cyclopropylethyl]-2-[(4S)-2,5-dioxo-2′,3′-dihydro-1H- |
| | spiro[imidazolidine-4,1′-inden]-1-yl]-N-(4-fluorobenzyl)acetamide |
| 493 | 2-(7′-bromo-1′-methyl-2,5-dioxo-2′,3′-dihydro-1H,1′H- |
| | spiro[imidazolidine-4,4′-quinolin]-1-yl)-N-[(1S)-1-cyclopropylethyl]-N-(4- |
| | fluorobenzyl)acetamide |
| 494 | N-(4-fluorobenzyl)-2-{6-[(methylcarbamoyl)amino]-2′,4′-dioxo-3,4- |
| | dihydro-2H,3′H-spiro[naphthalene-1,5′-[1,3]ozazolidin]-3′-yl}-N-[(2S)- |
| | 1,1,1-trifluoropropan-2-yl]acetamide |
| 495 | 2-[5′-(acetylamino)-2,5-dioxo-2′,3′-dihydro-1H-spiro[imidazolidine-4,1′- |
| | inden]-1-yl]-N-(4-fluorobenzyl)-N-[(2S)-1,1,1-trifluoropropan-2- |
| | yl]acetamide |
| 496 | N-[(1S)-1-cyclopropylethyl]-2-{(1R)-5-[(methylcarbamoyl)amino]-2′,4′- |
| | dioxo-2,3-dihydro-3′H-spiro[indene-1,5′-[1,3]ozazolidin]-3′-yl}-N- |
| | (pyrimidin-5-ylmethyl)acetamide |
| 497 | N-[(1S)-1-cyclopropylethyl]-2-{(1R)-5-[(methylcarbamoyl)amino]-2′,4′- |
| | dioxo-2,3-dihydro-3′H-spiro[indene-1,5′-[1,3]ozazolidin]-3′-yl}-N- |
| | (pyrimidin-2-ylmethyl)acetamide |
| 498 | N-[(1S)-1-cyclopropylethyl]-2-{(1R)-5-[(methylcarbamoyl)amino]-2′,4′- |
| | dioxo-2,3-dihydro-3′H-spiro[indene-1,5′-[1,3]ozazolidin]-3′-yl}-N-(1,3- |
| | thiazol-5-ylmethyl)acetamide |
| 499 | N-(4-fluorobenzyl)-2-(4′-hydroxy-2,5-dioxo-3′,4′-dihydro-1H,2′H- |
| | spiro[imidazolidine-4,1′-naphthalen]-1-yl)-N-[(2S)-1,1,1-trifluoropropan- |
| | 2-yl]acetamide |
| 500 | 2-(6-{1-[2-(dimethylamino)-2-oxoethyl]-1H-pyrazol-4-yl}-1,1-dioxido- |
| | 2′,5′-dioxo-1′H-spiro[1-benzothiophene-3,4′-imidazolidin]-1′-yl)-N-(4- |
| | fluorobenzyl)-N-[(2S)-1,1,1-trifluoropropan-2-yl]acetamide |
| 501 | 2-[5′-(acetylamino)-2,3′,5-trioxo-2′,3′-dihydro-1H-spiro[imidazolidine- |
| | 4,1′-inden]-1-yl]-N-(4-fluorobenzyl)-N-[(2S)-1,1,1-trifluoropropan-2- |
| | yl]acetamide |
| 502 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]prolinamide |
| 503 | N²-(4-fluorobenzyl)-N²-({(1R)-5-[(methylcarbamoyl)amino]-2′,4′-dioxo- |
| | 2,3-dihydro-3′H-spiro[indene-1,5′-[1,3]ozazolidin]-3′-yl}acetyl)-L- |
| | alaninamide |
| 504 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5-yl]- |
| | 1H-pyrazole-5-carboxamide |
| 505 | 2-[5′-(acetylamino)-2,5-dioxo-2′,3′-dihydro-1H-spiro[imidazolidine-4,1′- |
| | inden]-1-yl]-N-[(1S)-1-cyclopropylethyl]-N-(4-fluorobenzyl)acetamide |
| 506 | N-(4-fluorobenzyl)-2-[(1R)-5-{[(methylsulfamoyl)acetyl]amino}-2′,4′- |
| | dioxo-2,3-dihydro-3′H-spiro[indene-1,5′-[1,3]ozazolidin]-3′-yl]-N-[(2S)- |
| | 1,1,1-trifluoropropan-2-yl]acetamide |
| 507 | N-(4-fluorobenzyl)-2-[(1R)-5-{[(methylsulfonyl)acetyl]amino}-2′,4′- |
| | dioxo-2,3-dihydro-3′H-spiro[indene-1,5′-[1,3]ozazolidin]-3′-yl]-N-[(2S)- |
| | 1,1,1-trifluoropropan-2-yl]acetamide |
| 508 | 2-[(1R)-5-{[(dimethylsulfamoyl)acetyl]amino}-2′,4′-dioxo-2,3-dihydro- |
| | 3′H-spiro[indene-1,5′-[1,3]ozazolidin]-3′-yl]-N-(4-fluorobenzyl)-N-[(2S)- |
| | 1,1,1-trifluoropropan-2-yl]acetamide |
| 509 | 2-{(1R)-2′,4′-dioxo-5-[(sulfamoylacetyl)amino]-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 510 | N-(4-fluorobenzyl)-2-(5′-{1-[2-(methylamino)-2-oxoethyl]-1H-pyrazol-4- |
| | yl}-2,3′,5-trioxo-2′,3′-dihydro-1H-spiro[imidazolidine-4,1′-inden]-1-yl)-N- |
| | [(2S)-1,1,1-trifluoropropan-2-yl]acetamide |
| 511 | (3R)-3-[(4-fluorobenzyl)({(1R)-5-[(methylcarbamoyl)amino]-2′,4′-dioxo- |
| | 2,3-dihydro-3′H-spiro[indene-1,5′-[1,3]ozazolidin]-3′- |
| | yl}acetyl)amino]pyrrolidine-1-carboxamide |
| 512 | N-benzyl-N-ethyl-2-{(1R)-5-[(methylcarbamoyl)amino]-2′,4′-dioxo-2,3- |
| | dihydro-3′H-spiro[indene-1,5′-[1,3]ozazolidin]-3′-yl}acetamide |
| 513 | N-(4-fluorobenzyl)-2-(3′-hydroxy-5′-{1-[2-(methylamino)-2-oxoethyl]- |
| | 1H-pyrazol-4-yl}-2,5-dioxo-2′,3′-dihydro-1H-spiro[imidazolidine-4,1′- |
| | inden]-1-yl)-N-[(2S)-1,1,1-trifluoropropan-2-yl]acetamide |
| 514 | N-(4-fluorobenzyl)-2-(3′-fluoro-5′-{1-[2-(methylamino)-2-oxoethyl]-1H- |
| | pyrazol-4-yl}-2,5-dioxo-2′,3′-dihydro-1H-spiro[imidazolidine-4,1′-inden]- |
| | 1-yl)-N-[(2S)-1,1,1-trifluoropropan-2-yl]acetamide |
| 515 | 2-(6′-bromo-2,5-dioxo-3′,4′-dihydro-1H,1′H-spiro[imidazolidine-4,2′- |
| | naphthalen]-1-yl)-N-[(1S)-1-cyclopropylethyl]-N-(4- |
| | fluorobenzyl)acetamide |
| 516 | methyl N′-cyano-N-[(1R)-3′-(2-{(4-fluorobenzyl)[(2S)-1,1,1- |
| | trifluoropropan-2-yl]amino}-2-oxoethyl)-2′,4′-dioxo-2,3- |
| | dihydrospiro[indene-1,5′-[1,3]ozazolidin]-5-yl]carbamimidothioate |
| 517 | 3-[(4-fluorobenzyl)({(1R)-5-[(methylcarbamoyl)amino]-2′,4′-dioxo-2,3- |
| | dihydro-3′H-spiro[indene-1,5′-[1,3]ozazolidin]-3′- |
| | yl}acetyl)amino]butanamide |
| 518 | 2-(6′-amino-2,5-dioxo-3′,4′-dihydro-1H,1′H-spiro[imidazolidine-4,2′- |
| | naphthalen]-1-yl)-N-[(1S)-1-cyclopropylethyl]-N-(4- |
| | fluorobenzyl)acetamide |
| 519 | N-(4-fluorobenzyl)-2-{(1R)-5-[(methylcarbamothioyl)amino]-2′,4′-dioxo- |
| | 2,3-dihydro-3′H-spiro[indene-1,5′-[1,3]ozazolidin]-3′-yl}-N-[(2S)-1,1,1- |
| | trifluoropropan-2-yl]acetamide |
| 520 | N-[(1S)-1-cyclopropylethyl]-2-{(1R)-5-[(methylcarbamoyl)amino]-2′,4′- |
| | dioxo-2,3-dihydro-3′H-spiro[indene-1,5′-[1,3]ozazolidin]-3′-yl}-N- |
| | (tetrahydro-2H-pyran-4-ylmethyl)acetamide |
| 521 | N-[(1S)-1-cyclopropylethyl]-2-{(1R)-5-[(methylcarbamoyl)amino]-2′,4′- |
| | dioxo-2,3-dihydro-3′H-spiro[indene-1,5′-[1,3]ozazolidin]-3′-yl}-N-(1,3- |
| | oxazol-4-ylmethyl)acetamide |
| 522 | 2-(7′-amino-1′-methyl-2,5-dioxo-2′,3′-dihydro-1H,1′H-spiro[imidazolidine- |
| | 4,4′-quinolin]-1-yl)-N-[(1S)-1-cyclopropylethyl]-N-(4- |
| | fluorobenzyl)acetamide |
| 523 | 2-[(1R)-5-(5-cyanothiophen-2-yl)-2′,4′-dioxo-2,3-dihydro-3′H- |
| | spiro[indene-1,5′-[1,3]ozazolidin]-3′-yl]-N-(4-fluorobenzyl)-N-[(2S)-1,1,1- |
| | trifluoropropan-2-yl]acetamide |
| 524 | 2-[(1R)-5-(4-cyano-3-fluorophenyl)-2′,4′-dioxo-2,3-dihydro-3′H- |
| | spiro[indene-1,5′-[1,3]ozazolidin]-3′-yl]-N-(4-fluorobenzyl)-N-[(2S)-1,1,1- |
| | trifluoropropan-2-yl]acetamide |
| 525 | 2-[(1R)-5-(5-cyanothiophen-3-yl)-2′,4′-dioxo-2,3-dihydro-3′H- |
| | spiro[indene-1,5′-[1,3]ozazolidin]-3′-yl]-N-(4-fluorobenzyl)-N-[(2S)-1,1,1- |
| | trifluoropropan-2-yl]acetamide |
| 526 | N-[(1S)-1-cyclopropylethyl]-N-(4-fluorobenzyl)-2-{1′-methyl-7′- |
| | [(methylcarbamoyl)amino]-2,5-dioxo-2′,3′-dihydro-1H,1′H- |
| | spiro[imidazolidine-4,4′-quinolin]-1-yl}acetamide |
| 527 | N-[(1S)-1-cyclopropylethyl]-N-(4-fluorobenzyl)-2-{6′- |
| | [(methylcarbamoyl)amino]-2,5-dioxo-3′,4′-dihydro-1H,1′H- |
| | spiro[imidazolidine-4,2′-naphthalen]-1-yl}acetamide |
| 528 | 2-[6′-(acetylamino)-2,5-dioxo-3′,4′-dihydro-1H,1′H-spiro[imidazolidine- |
| | 4,2′-naphthalen]-1-yl]-N-[(1S)-1-cyclopropylethyl]-N-(4- |
| | fluorobenzyl)acetamide |
| 529 | 2-[(1R)-5-(N″-cyano-N′-methylcarbamimidamido)-2′,4′-dioxo-2,3- |
| | dihydro-3′H-spiro[indene-1,5′-[1,3]ozazolidin]-3′-yl]-N-(4-fluorobenzyl)- |
| | N-[(2S)-1,1,1-trifluoropropan-2-yl]acetamide |
| 530 | 2-[(1R)-5-(4,6-difluoropyrimidin-5-yl)-2′,4′-dioxo-2,3-dihydro-3′H- |
| | spiro[indene-1,5′-[1,3]ozazolidin]-3′-yl]-N-(4-fluorobenzyl)-N-[(2S)-1,1,1- |
| | trifluoropropan-2-yl]acetamide |
| 531 | 2-{(1R)-5-[2-(acetylamino)-5-methylpyridin-3-yl]-2′,4′-dioxo-2,3-dihydro- |
| | 3′H-spiro[indene-1,5′-[1,3]ozazolidin]-3′-yl}-N-(4-fluorobenzyl)-N-[(2S)- |
| | 1,1,1-trifluoropropan-2-yl]acetamide |
| 532 | N-(4-fluorobenzyl)-2-[(1R)-5-(5-fluoropyridin-3-yl)-2′,4′-dioxo-2,3- |
| | dihydro-3′H-spiro[indene-1,5′-[1,3]ozazolidin]-3′-yl]-N-[(2S)-1,1,1- |
| | trifluoropropan-2-yl]acetamide |
| 533 | N-(4-fluorobenzyl)-2-[(1R)-5-(furan-3-yl)-2′,4′-dioxo-2,3-dihydro-3′H- |
| | spiro[indene-1,5′-[1,3]ozazolidin]-3′-yl]-N-[(2S)-1,1,1-trifluoropropan-2- |
| | yl]acetamide |
| 534 | 2-[(1R)-2′,4′-dioxo-5-(2-oxo-2,3-dihydro-1H-benzimidazol-5-yl)-2,3- |
| | dihydro-3′H-spiro[indene-1,5′-[1,3]ozazolidin]-3′-yl]-N-(4-fluorobenzyl)- |
| | N-[(2S)-1,1,1-trifluoropropan-2-yl]acetamide |
| 535 | N-(4-fluorobenzyl)-2-[(1R)-5-(4-methylpyrimidin-2-yl)-2′,4′-dioxo-2,3- |
| | dihydro-3′H-spiro[indene-1,5′-[1,3]ozazolidin]-3′-yl]-N-[(2S)-1,1,1- |
| | trifluoropropan-2-yl]acetamide |
| 536 | 2-{(1R)-2′,4′-dioxo-5-[1-(tetrahydro-2H-pyran-2-yl)-1H-pyrazol-3-yl]-2,3- |
| | dihydro-3′H-spiro[indene-1,5′-[1,3]ozazolidin]-3′-yl}-N-(4-fluorobenzyl)- |
| | N-[(2S)-1,1,1-trifluoropropan-2-yl]acetamide |
| 537 | N-(4-fluorobenzyl)-2-[(1R)-5-(5-fluoro-6-methylpyridin-2-yl)-2′,4′-dioxo- |
| | 2,3-dihydro-3′H-spiro[indene-1,5′-[1,3]ozazolidin]-3′-yl]-N-[(2S)-1,1,1- |
| | trifluoropropan-2-yl]acetamide |
| 538 | 2-{(1R)-5-[6-(difluoromethyl)pyridin-3-yl]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 539 | 2-{(1R)-5-[6-(difluoromethyl)pyridin-2-yl]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 540 | N-(4-fluorobenzyl)-2-[(1R)-5-(furan-2-yl)-2′,4′-dioxo-2,3-dihydro-3′H- |
| | spiro[indene-1,5′-[1,3]ozazolidin]-3′-yl]-N-[(2S)-1,1,1-trifluoropropan-2- |
| | yl]acetamide |
| 541 | 2-{(1R)-5-[1-(ethoxymethyl)-1H-imidazol-2-yl]-2′,4′-dioxo-2,3-dihydro- |
| | 3′H-spiro[indene-1,5′-[1,3]ozazolidin]-3′-yl}-N-(4-fluorobenzyl)-N-[(2S)- |
| | 1,1,1-trifluoropropan-2-yl]acetamide |
| 542 | 2-[(1R)-5-(5-cyano-1,2-dimethyl-6-oxo-1,6-dihydropyridin-3-yl)-2′,4′- |
| | dioxo-2,3-dihydro-3′H-spiro[indene-1,5′-[1,3]ozazolidin]-3′-yl]-N-(4- |
| | fluorobenzyl)-N-[(2S)-1,1,1-trifluoropropan-2-yl]acetamide |
| 543 | 3-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]thiophene-2-carboxamide |
| 544 | 2-[(1R)-5-(2,6-dioxo-1,2,5,6-tetrahydropyrimidin-5-yl)-2′,4′-dioxo-2,3- |
| | dihydro-3′H-spiro[indene-1,5′-[1,3]ozazolidin]-3′-yl]-N-(4-fluorobenzyl)- |
| | N-[(2S)-1,1,1-trifluoropropan-2-yl]acetamide |
| 545 | 2-[(1R)-5-(2,4-dimethyl-1,3-thiazol-5-yl)-2′,4′-dioxo-2,3-dihydro-3′H- |
| | spiro[indene-1,5′-[1,3]ozazolidin]-3′-yl]-N-(4-fluorobenzyl)-N-[(2S)-1,1,1- |
| | trifluoropropan-2-yl]acetamide |
| 546 | 2-{(1R)-5-[1-(cyanomethyl)-3,5-dimethyl-1H-pyrazol-4-yl]-2′,4′-dioxo- |
| | 2,3-dihydro-3′H-spiro[indene-1,5′-[1,3]ozazolidin]-3′-yl}-N-(4- |
| | fluorobenzyl)-N-[(2S)-1,1,1-trifluoropropan-2-yl]acetamide |
| 547 | 2-{(1R)-5-[3-(carbamoylamino)phenyl]-2′,4′-dioxo-2,3-dihydro-3′H- |
| | spiro[indene-1,5′-[1,3]ozazolidin]-3′-yl}-N-(4-fluorobenzyl)-N-[(2S)- |
| | 1,1,1-trifluoropropan-2-yl]acetamide |
| 548 | N-[3-({[(1R)-3′-(2-{(4-fluorobenzyl)[(2S)-1,1,1-trifluoropropan-2- |
| | yl]amino}-2-oxoethyl)-2′,4′-dioxo-2,3-dihydrospiro[indene-1,5′- |
| | [1,3]ozazolidin]-5-yl]carbamoyl}amino)-2,2-dimethylpropyl]propanamide |
| 549 | (1R)-3′-(2-{benzyl[(1S)-1-cyclopropylethyl]amino}-2-oxoethyl)-N- |
| | methyl-2′,4′-dioxo-2,3-dihydrospiro[indene-1,5′-[1,3]ozazolidine]-5- |
| | carboxamide |
| 550 | (1R)-3′-(2-{benzyl[(1S)-1-cyclopropylethyl]amino}-2-oxoethyl)-2′,4′- |
| | dioxo-N-(propan-2-yl)-2,3-dihydrospiro[indene-1,5′-[1,3]ozazolidine]-5- |
| | carboxamide |
| 551 | (1R)-3′-(2-{benzyl[(1S)-1-cyclopropylethyl]amino}-2-oxoethyl)-N-(2- |
| | methoxyethyl)-2′,4′-dioxo-2,3-dihydrospiro[indene-1,5′-[1,3]ozazolidine]- |
| | 5-carboxamide |
| 552 | (1R)-3′-(2-{benzyl[(1S)-1-cyclopropylethyl]amino}-2-oxoethyl)-2′,4′- |
| | dioxo-N-(tetrahydrofuran-2-ylmethyl)-2,3-dihydrospiro[indene-1,5′- |
| | [1,3]ozazolidine]-5-carboxamide |
| 553 | (1R)-3′-(2-{benzyl[(1S)-1-cyclopropylethyl]amino}-2-oxoethyl)-2′,4′- |
| | dioxo-N-(tetrahydrofuran-3-ylmethyl)-2,3-dihydrospiro[indene-1,5′- |
| | [1,3]ozazolidine]-5-carboxamide |
| 554 | (1R)-3′-(2-{benzyl[(1S)-1-cyclopropylethyl]amino}-2-oxoethyl)-N- |
| | (cyanomethyl)-2′,4′-dioxo-2,3-dihydrospiro[indene-1,5′-[1,3]ozazolidine]- |
| | 5-carboxamide |
| 555 | (1R)-3′-(2-{benzyl[(1S)-1-cyclopropylethyl]amino}-2-oxoethyl)-N-(3- |
| | hydroxypropyl)-2′,4′-dioxo-2,3-dihydrospiro[indene-1,5′- |
| | [1,3]ozazolidine]-5-carboxamide |
| 556 | 3′-{2-[benzyl(1-cyclopropylethyl)amino]-2-oxoethyl}-N-(1-hydroxy-3- |
| | methylbutan-2-yl)-2′,4′-dioxo-2,3-dihydrospiro[indene-1,5′- |
| | [1,3]ozazolidine]-5-carboxamide |
| 557 | N-(1-amino-4-methyl-1-oxopentan-2-yl)-3′-{2-[benzyl(1- |
| | cyclopropylethyl)amino]-2-oxoethyl}-2′,4′-dioxo-2,3-dihydrospiro[indene- |
| | 1,5′-[1,3]ozazolidine]-5-carboxamide |
| 558 | (1R)-3′-(2-{benzyl[(1S)-1-cyclopropylethyl]amino}-2-oxoethyl)-N-[2- |
| | (diethylamino)ethyl]-2′,4′-dioxo-2,3-dihydrospiro[indene-1,5′- |
| | [1,3]ozazolidine]-5-carboxamide |
| 559 | (1R)-3′-(2-{benzyl[(1S)-1-cyclopropylethyl]amino}-2-oxoethyl)-N-[3- |
| | (1H-imidazol-1-yl)propyl]-2′,4′-dioxo-2,3-dihydrospiro[indene-1,5′- |
| | [1,3]ozazolidine]-5-carboxamide |
| 560 | (1R)-3′-(2-{benzyl[(1S)-1-cyclopropylethyl]amino}-2-oxoethyl)-2′,4′- |
| | dioxo-N-[3-(2-oxopyrrolidin-1-yl)propyl]-2,3-dihydrospiro[indene-1,5′- |
| | [1,3]ozazolidine]-5-carboxamide |
| 561 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5- |
| | yl]tetrahydropyrimidine-1(2H)-carboxamide |
| 562 | 3-acetyl-N-[(1R)-3′-(2-{(4-fluorobenzyl)[(2S)-1,1,1-trifluoropropan-2- |
| | yl]amino}-2-oxoethyl)-2′,4′-dioxo-2,3-dihydrospiro[indene-1,5′- |
| | [1,3]ozazolidin]-5-yl]tetrahydropyrimidine-1(2H)-carboxamide |
| 563 | 2-(6-amino-1,1-dioxido-2′,5′-dioxo-1′H-spiro[1-benzothiophene-3,4′- |
| | imidazolidin]-1′-yl)-N-(4-fluorobenzyl)-N-[(2S)-1,1,1-trifluoropropan-2- |
| | yl]acetamide |
| 564 | N-(4-fluorobenzyl)-2-(6-{1-[2-(methylamino)-2-oxoethyl]-1H-pyrazol-4- |
| | yl}-1,1-dioxido-2′,5′-dioxo-1′H-spiro[1-benzothiophene-3,4′- |
| | imidazolidin]-1′-yl)-N-[(2S)-1,1,1-trifluoropropan-2-yl]acetamide |
| 565 | N-(4-fluorobenzyl)-2-{(1R)-5-[1-(2-hydroxyethyl)-1H-pyrazol-4-yl]-2′,4′- |
| | dioxo-2,3-dihydro-3′H-spiro[indene-1,5′-[1,3]ozazolidin]-3′-yl}-N-[(2S)- |
| | 1,1,1-trifluoropropan-2-yl]acetamide |
| 566 | N-benzyl-2-{(1R)-5-[(methylcarbamoyl)amino]-2′,4′-dioxo-2,3-dihydro- |
| | 3′H-spiro[indene-1,5′-[1,3]ozazolidin]-3′-yl}-N-phenylacetamide |
| 567 | N-benzyl-2-{(1R)-5-[(methylcarbamoyl)amino]-2′,4′-dioxo-2,3-dihydro- |
| | 3′H-spiro[indene-1,5′-[1,3]ozazolidin]-3′-yl}-N-[(1S)-1- |
| | phenylethyl]acetamide |
| 568 | N,N-dibenzyl-2-{(1R)-5-[(methylcarbamoyl)amino]-2′,4′-dioxo-2,3- |
| | dihydro-3′H-spiro[indene-1,5′-[1,3]ozazolidin]-3′-yl}acetamide |
| 569 | tert-butyl {4-[1-(2-{(4-fluorobenzyl)[(2S)-1,1,1-trifluoropropan-2- |
| | yl]amino}-2-oxoethyl)-2,3′,5-trioxo-2′,3′-dihydrospiro[imidazolidine-4,1′- |
| | inden]-5′-yl]-1H-pyrazol-1-yl}acetate |
| 570 | N-(4-fluorobenzyl)-2-{3′-fluoro-5′-[1-(2-methylpropyl)-1H-pyrazol-4-yl]- |
| | 2,5-dioxo-2′,3′-dihydro-1H-spiro[imidazolidine-4,1′-inden]-1-yl}-N-[(2S)- |
| | 1,1,1-trifluoropropan-2-yl]acetamide |
| 571 | {4-[1-(2-{(4-fluorobenzyl)[(2S)-1,1,1-trifluoropropan-2-yl]amino}-2- |
| | oxoethyl)-2,3′,5-trioxo-2′,3′-dihydrospiro[imidazolidine-4,1′-inden]-5′-yl]- |
| | 1H-pyrazol-1-yl}acetic acid |
| 572 | N-benzyl-N-(cyclopropylmethyl)-2-{(1R)-5-[(methylcarbamoyl)amino]- |
| | 2′,4′-dioxo-2,3-dihydro-3′H-spiro[indene-1,5′-[1,3]ozazolidin]-3′- |
| | yl}acetamide |
| 573 | (2R)-3,3,3-trifluoro-N-[(1R)-3′-(2-{(4-fluorobenzyl)[(2S)-1,1,1- |
| | trifluoropropan-2-yl]amino}-2-oxoethyl)-2′,4′-dioxo-2,3- |
| | dihydrospiro[indene-1,5′-[1,3]ozazolidin]-5-yl]-2-hydroxy-2- |
| | methylpropanamide |
| 574 | (2S)-3,3,3-trifluoro-N-[(1R)-3′-(2-{(4-fluorobenzyl)[(2S)-1,1,1- |
| | trifluoropropan-2-yl]amino}-2-oxoethyl)-2′,4′-dioxo-2,3- |
| | dihydrospiro[indene-1,5′-[1,3]ozazolidin]-5-yl]-2-hydroxy-2- |
| | methylpropanamide |
| 575 | 2-[(1R)-2′,4′-dioxo-5-({[3-(pyrrolidin-1- |
| | ylmethyl)phenyl]carbamoyl}amino)-2,3-dihydro-3′H-spiro[indene-1,5′- |
| | [1,3]ozazolidin]-3′-yl]-N-(4-fluorobenzyl)-N-[(2S)-1,1,1-trifluoropropan- |
| | 2-yl]acetamide |
| 576 | N-(4-fluorobenzyl)-2-[(1R)-5-({[3-(morpholin-4- |
| | ylmethyl)phenyl]carbamoyl}amino)-2′,4′-dioxo-2,3-dihydro-3′H- |
| | spiro[indene-1,5′-[1,3]ozazolidin]-3′-yl]-N-[(2S)-1,1,1-trifluoropropan-2- |
| | yl]acetamide |
| 577 | N³-{[(1R)-3′-(2-{(4-fluorobenzyl)[(2S)-1,1,1-trifluoropropan-2-yl]amino}- |
| | 2-oxoethyl)-2′,4′-dioxo-2,3-dihydrospiro[indene-1,5′-[1,3]ozazolidin]-5- |
| | yl]carbamoyl}-N-methyl-beta-alaninamide |
| 578 | N-[3-({[(1R)-3′-(2-{(4-fluorobenzyl)[(2S)-1,1,1-trifluoropropan-2- |
| | yl]amino}-2-oxoethyl)-2′,4′-dioxo-2,3-dihydrospiro[indene-1,5′- |
| | [1,3]ozazolidin]-5-yl]carbamoyl}amino)phenyl]propanamide |
| 579 | 2-(5′-{1-[2-(dimethylamino)-2-oxoethyl]-1H-pyrazol-4-yl}-2,3′,5-trioxo- |
| | 2′,3′-dihydro-1H-spiro[imidazolidine-4,1′-inden]-1-yl)-N-(4-fluorobenzyl)- |
| | N-[(2S)-1,1,1-trifluoropropan-2-yl]acetamide |
| 580 | N-(4-fluorobenzyl)-2-(5′-{1-[2-(3-hydroxyazetidin-1-yl)-2-oxoethyl]-1H- |
| | pyrazol-4-yl}-2,3′,5-trioxo-2′,3′-dihydro-1H-spiro[imidazolidine-4,1′- |
| | inden]-1-yl)-N-[(2S)-1,1,1-trifluoropropan-2-yl]acetamide |
| 581 | 2-(6-bromo-2′,4,4′-trioxo-3,4-dihydro-2H,3′H-spiro[naphthalene-1,5′- |
| | [1,3]ozazolidin]-3′-yl)-N-[(1S)-1-cyclopropyl-2,2,2-trifluoroethyl]-N-(4- |
| | fluorobenzyl)acetamide |
| 582 | N-(4-fluorobenzyl)-2-{6-[(methylcarbamoyl)amino]-1,1-dioxido-2′,5′- |
| | dioxo-1′H-spiro[1-benzothiophene-3,4′-imidazolidin]-1′-yl}-N-[(2S)-1,1,1- |
| | trifluoropropan-2-yl]acetamide |
| 583 | N-(2-chlorobenzyl)-2-{(1R)-5-[(methylcarbamoyl)amino]-2′,4′-dioxo-2,3- |
| | dihydro-3′H-spiro[indene-1,5′-[1,3]ozazolidin]-3′-yl}-N-(2- |
| | methylpropyl)acetamide |
| 584 | N-[(1S)-1-cyclopropyl-2,2,2-trifluoroethyl]-N-(4-fluorobenzyl)-2-{6- |
| | [(methylcarbamoyl)amino]-2′,4,4′-trioxo-3,4-dihydro-2H,3′H- |
| | spiro[naphthalene-1,5′-[1,3]ozazolidin]-3′-yl}acetamide |
| 585 | N-(4-fluorobenzyl)-2-{(1S)-6-[(methylcarbamoyl)amino]-2′,4′-dioxo-3,4- |
| | dihydro-2H,3′H-spiro[naphthalene-1,5′-[1,3]ozazolidin]-3′-yl}-N-[(2S)- |
| | 1,1,1-trifluoropropan-2-yl]acetamide |
| 586 | N-(4-fluorobenzyl)-2-{(1R)-6-[(methylcarbamoyl)amino]-2′,4′-dioxo-3,4- |
| | dihydro-2H,3′H-spiro[naphthalene-1,5′-[1,3]ozazolidin]-3′-yl}-N-[(2S)- |
| | 1,1,1-trifluoropropan-2-yl]acetamide |
| 587 | 2-[(1R)-5-(4,5-dihydro-1H-imidazol-2-ylamino)-2′,4′-dioxo-2,3-dihydro- |
| | 3′H-spiro[indene-1,5′-[1,3]ozazolidin]-3′-yl]-N-(4-fluorobenzyl)-N-[(2S)- |
| | 1,1,1-trifluoropropan-2-yl]acetamide |
| 588 | 2-(6-amino-2′,4′-dioxo-3,4-dihydro-2H,3′H-spiro[naphthalene-1,5′- |
| | [1,3]ozazolidin]-3′-yl)-N-[(1S)-1-cyclopropyl-2,2,2-trifluoroethyl]-N-(4- |
| | fluorobenzyl)acetamide |
| 589 | 2-[(1R)-5-({[3-(acetylamino)propyl]carbamoyl}amino)-2′,4′-dioxo-2,3- |
| | dihydro-3′H-spiro[indene-1,5′-[1,3]ozazolidin]-3′-yl]-N-(4-fluorobenzyl)- |
| | N-[(2S)-1,1,1-trifluoropropan-2-yl]acetamide |
| 590 | 2-[(1R)-5-({[2-(acetylamino)ethyl]carbamoyl}amino)-2′,4′-dioxo-2,3- |
| | dihydro-3′H-spiro[indene-1,5′-[1,3]ozazolidin]-3′-yl]-N-(4-fluorobenzyl)- |
| | N-[(2S)-1,1,1-trifluoropropan-2-yl]acetamide |
| 591 | tert-butyl [3-({[(1R)-3′-(2-{(4-fluorobenzyl)[(2S)-1,1,1-trifluoropropan-2- |
| | yl]amino}-2-oxoethyl)-2′,4′-dioxo-2,3-dihydrospiro[indene-1,5′- |
| | [1,3]ozazolidin]-5-yl]carbamoyl}amino)-2,2-dimethylpropyl]carbamate |
| 592 | N-[2-({[(1R)-3′-(2-{(4-fluorobenzyl)[(2S)-1,1,1-trifluoropropan-2- |
| | yl]amino}-2-oxoethyl)-2′,4′-dioxo-2,3-dihydrospiro[indene-1,5′- |
| | [1,3]ozazolidin]-5-yl]carbamoyl}amino)ethyl]propanamide |
| 593 | 2-[(1R)-5-{[(3-amino-2,2-dimethylpropyl)carbamoyl]amino}-2′,4′-dioxo- |
| | 2,3-dihydro-3′H-spiro[indene-1,5′-[1,3]ozazolidin]-3′-yl]-N-(4- |
| | fluorobenzyl)-N-[(2S)-1,1,1-trifluoropropan-2-yl]acetamide |
| 594 | 2-[(1R)-5-({[3-(acetylamino)phenyl]carbamoyl}amino)-2′,4′-dioxo-2,3- |
| | dihydro-3′H-spiro[indene-1,5′-[1,3]ozazolidin]-3′-yl]-N-(4-fluorobenzyl)- |
| | N-[(2S)-1,1,1-trifluoropropan-2-yl]acetamide |
| 595 | methyl 2-{[(1R)-3′-(2-{(4-fluorobenzyl)[(2S)-1,1,1-trifluoropropan-2- |
| | yl]amino}-2-oxoethyl)-2′,4′-dioxo-2,3-dihydrospiro[indene-1,5′- |
| | [1,3]ozazolidin]-5-yl]amino}-4,5-dihydro-1H-imidazole-1-carboxylate |
| 596 | 2-{5-bromo-6-[(methylcarbamoyl)amino]-2′,4′-dioxo-3,4-dihydro-2H,3′H- |
| | spiro[naphthalene-1,5′-[1,3]ozazolidin]-3′-yl}-N-[(1S)-1-cyclopropyl- |
| | 2,2,2-trifluoroethyl]-N-(4-fluorobenzyl)acetamide |
| 597 | 2-(5′-{1-[2-(dimethylamino)-2-oxoethyl]-1H-pyrazol-4-yl}-3′-fluoro-2,5- |
| | dioxo-2′,3′-dihydro-1H-spiro[imidazolidine-4,1′-inden]-1-yl)-N-(4- |
| | fluorobenzyl)-N-[(2S)-1,1,1-trifluoropropan-2-yl]acetamide |
| 598 | 2-{5′-[1-(2-amino-2-oxoethyl)-1H-pyrazol-4-yl]-3′-fluoro-2,5-dioxo-2′,3′- |
| | dihydro-1H-spiro[imidazolidine-4,1′-inden]-1-yl}-N-(4-fluorobenzyl)-N- |
| | [(2S)-1,1,1-trifluoropropan-2-yl]acetamide |
| 599 | N-cyclohexyl-N-(4-fluorobenzyl)-2-{(1R)-5-[(methylcarbamoyl)amino]- |
| | 2′,4′-dioxo-2,3-dihydro-3′H-spiro[indene-1,5′-[1,3]ozazolidin]-3′- |
| | yl}acetamide |
| 600 | N-cyclopentyl-N-(4-fluorobenzyl)-2-{(1R)-5-[(methylcarbamoyl)amino]- |
| | 2′,4′-dioxo-2,3-dihydro-3′H-spiro[indene-1,5′-[1,3]ozazolidin]-3′- |
| | yl}acetamide |
| 601 | N-[(1S)-1-cyclopropyl-2,2,2-trifluoroethyl]-N-(4-fluorobenzyl)-2-{4- |
| | hydroxy-6-[(methylcarbamoyl)amino]-2′,4′-dioxo-3,4-dihydro-2H,3′H- |
| | spiro[naphthalene-1,5′-[1,3]ozazolidin]-3′-yl}acetamide |
| 602 | N-benzyl-N-[1-(furan-2-yl)ethyl]-2-{(1R)-5-[(methylcarbamoyl)amino]- |
| | 2′,4′-dioxo-2,3-dihydro-3′H-spiro[indene-1,5′-[1,3]ozazolidin]-3′- |
| | yl}acetamide |
| 603 | N-benzyl-N-cyclobutyl-2-{(1R)-5-[(methylcarbamoyl)amino]-2′,4′-dioxo- |
| | 2,3-dihydro-3′H-spiro[indene-1,5′-[1,3]ozazolidin]-3′-yl}acetamide |
| 604 | N-[(1S)-1-cyclopropyl-2,2,2-trifluoroethyl]-N-(4-fluorobenzyl)-2-{4- |
| | fluoro-6-[(methylcarbamoyl)amino]-2′,4′-dioxo-3,4-dihydro-2H,3′H- |
| | spiro[naphthalene-1,5′-[1,3]ozazolidin]-3′-yl}acetamide |
| 605 | 2-{5′-[1-(2-amino-2-oxoethyl)-1H-pyrazol-4-yl]-3′-hydroxy-2,5-dioxo- |
| | 2′,3′-dihydro-1H-spiro[imidazolidine-4,1′-inden]-1-yl}-N-(4- |
| | fluorobenzyl)-N-[(2S)-1,1,1-trifluoropropan-2-yl]acetamide |
| 606 | N-{[(1R)-3′-(2-{(4-fluorobenzyl)[(2S)-1,1,1-trifluoropropan-2-yl]amino}- |
| | 2-oxoethyl)-2′,4′-dioxo-2,3-dihydrospiro[indene-1,5′-[1,3]ozazolidin]-5- |
| | yl]carbamoyl}-beta-alanine |
| 607 | N-cyclopropyl-N-(4-fluorobenzyl)-2-{(1R)-5-[(methylcarbamoyl)amino]- |
| | 2′,4′-dioxo-2,3-dihydro-3′H-spiro[indene-1,5′-[1,3]ozazolidin]-3′- |
| | yl}acetamide |
| 608 | N-(3,5-difluorobenzyl)-2-{(1R)-5-[(methylcarbamoyl)amino]-2′,4′-dioxo- |
| | 2,3-dihydro-3′H-spiro[indene-1,5′-[1,3]ozazolidin]-3′-yl}-N-[(2S)-1,1,1- |
| | trifluoropropan-2-yl]acetamide |
| 609 | N-(4-fluorobenzyl)-N-(3-fluorophenyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 610 | 2-[(1S)-5-amino-2′,4′-dioxo-2,3-dihydro-3′H-spiro[indene-1,5′- |
| | [1,3]ozazolidin]-3′-yl]-N-[(1R)-1-cyclopropylethyl]-N-(4- |
| | fluorobenzyl)acetamide |
| 611 | 2-[(1R)-5-amino-2′,4′-dioxo-2,3-dihydro-3′H-spiro[indene-1,5′- |
| | [1,3]ozazolidin]-3′-yl]-N-(4-fluorobenzyl)-N-[(2S)-1,1,1-trifluoropropan- |
| | 2-yl]acetamide |
| 612 | ethyl ({(3S)-3-[(4-fluorobenzyl){[(1R)-5-(1-methyl-1H-pyrazol-4-yl)-2′,4′- |
| | dioxo-2,3-dihydro-3′H-spiro[indene-1,5′-[1,3]ozazolidin]-3′- |
| | yl]acetyl}amino]pyrrolidin-1-yl}sulfonyl)carbamate |
| 613 | 2-[(1R)-5-bromo-2′,4′-dioxo-2,3-dihydro-3′H-spiro[indene-1,5′- |
| | [1,3]ozazolidin]-3′-yl]-N-(4-fluorobenzyl)-N-[(3R)-piperidin-3- |
| | yl]acetamide |
| 614 | ethyl ({(3R)-3-[{[(1R)-5-bromo-2′,4′-dioxo-2,3-dihydro-3′H-spiro[indene- |
| | 1,5′-[1,3]ozazolidin]-3′-yl]acetyl}(4-fluorobenzyl)amino]piperidin-1- |
| | yl}sulfonyl)carbamate |
| 615 | N-(4-fluorobenzyl)-2-{(1R)-5-[(methylcarbamoyl)amino]-2′,4′-dioxo-2,3- |
| | dihydro-3′H-spiro[indene-1,5′-[1,3]ozazolidin]-3′-yl}-N-[1- |
| | (methylsulfonyl)piperidin-4-yl]acetamide |
| 616 | 2-[(4S)-5′-{1-[2-(dimethylamino)-2-oxoethyl]-1H-pyrazol-4-yl}-2,3′,5- |
| | trioxo-2′,3′-dihydro-1H-spiro[imidazolidine-4,1′-inden]-1-yl]-N-(4- |
| | fluorobenzyl)-N-[(2S)-1,1,1-trifluoropropan-2-yl]acetamide |
| 617 | 2-[(4S)-5′-{1-[2-(dimethylamino)-2-oxoethyl]-1H-pyrazol-4-yl}-2,5- |
| | dioxo-2′,3′-dihydro-1H-spiro[imidazolidine-4,1′-inden]-1-yl]-N-(4- |
| | fluorobenzyl)-N-[(2S)-1,1,1-trifluoropropan-2-yl]acetamide |
| 618 | N-(3,5-difluorobenzyl)-2-{(1R)-5-[(methylcarbamoyl)amino]-2′,4′-dioxo- |
| | 2,3-dihydro-3′H-spiro[indene-1,5′-[1,3]ozazolidin]-3′-yl}-N-[(2S)-1,1,1- |
| | trifluoropropan-2-yl]acetamide |
| 619 | N-(4-fluorobenzyl)-N-[(2S)-1,1,1-trifluoropropan-2-yl]-2-[(4R)-2,3′,5- |
| | trioxo-2′,3′-dihydro-1H-spiro[imidazolidine-4,1′-inden]-1-yl]acetamide |
| 620 | N-(4-fluorobenzyl)-N-[(2S)-1,1,1-trifluoropropan-2-yl]-2-[(4S)-2,3′,5- |
| | trioxo-2′,3′-dihydro-1H-spiro[imidazolidine-4,1′-inden]-1-yl]acetamide |
| 621 | N-(4-fluorobenzyl)-2-[5′-(1-methyl-1H-pyrazol-4-yl)-2,3′,5-trioxo-2′,3′- |
| | dihydro-1H-spiro[imidazolidine-4,1′-inden]-1-yl]-N-[(2S)-1,1,1- |
| | trifluoropropan-2-yl]acetamide |
| 622 | N-(4-fluorobenzyl)-2-{6-fluoro-5-[(methylcarbamoyl)amino]-2′,4′-dioxo- |
| | 2,3-dihydro-3′H-spiro[indene-1,5′-[1,3]ozazolidin]-3′-yl}-N-[(2S)-1,1,1- |
| | trifluoropropan-2-yl]acetamide |
| 623 | N-(4-fluorobenzyl)-2-[3′-hydroxy-5′-(1-methyl-1H-pyrazol-4-yl)-2,5- |
| | dioxo-2′,3′-dihydro-1H-spiro[imidazolidine-4,1′-inden]-1-yl]-N-[(2S)- |
| | 1,1,1-trifluoropropan-2-yl]acetamide |
| 624 | 2-(5-amino-6-fluoro-2′,4′-dioxo-2,3-dihydro-3′H-spiro[indene-1,5′- |
| | [1,3]ozazolidin]-3′-yl)-N-(4-fluorobenzyl)-N-[(2S)-1,1,1-trifluoropropan- |
| | 2-yl]acetamide |
| 625 | 2-(7′-bromo-1′-methyl-2,5-dioxo-2′,3′-dihydro-1H,1′H- |
| | spiro[imidazolidine-4,4′-quinolin]-1-yl)-N-[(1S)-1-cyclopropyl-2,2,2- |
| | trifluoroethyl]-N-(4-fluorobenzyl)acetamide |
| 626 | N-(cyclopropylmethyl)-2-{(1R)-5-[(methylcarbamoyl)amino]-2′,4′-dioxo- |
| | 2,3-dihydro-3′H-spiro[indene-1,5′-[1,3]ozazolidin]-3′-yl}-N-(1- |
| | phenylethyl)acetamide |
| 627 | 2-{(1R)-5-[(methylcarbamoyl)amino]-2′,4′-dioxo-2,3-dihydro-3′H- |
| | spiro[indene-1,5′-[1,3]ozazolidin]-3′-yl}-N-(2-methylpropyl)-N-[2- |
| | (trifluoromethyl)benzyl]acetamide |
| 628 | 2-(5-bromo-6-fluoro-2′,4′-dioxo-2,3-dihydro-3′H-spiro[indene-1,5′- |
| | [1,3]ozazolidin]-3′-yl)-N-(4-fluorobenzyl)-N-[(2S)-1,1,1-trifluoropropan- |
| | 2-yl]acetamide |
| 629 | N-(4-fluorobenzyl)-2-{1′-methyl-7′-[(methylcarbamoyl)amino]-2,5-dioxo- |
| | 2′,3′-dihydro-1H,1′H-spiro[imidazolidine-4,4′-quinolin]-1-yl}-N-[(2S)- |
| | 1,1,1-trifluoropropan-2-yl]acetamide |
| 630 | N-(4-fluorobenzyl)-2-[(3′R,4R)-3′-hydroxy-2,5-dioxo-2′,3′-dihydro-1H- |
| | spiro[imidazolidine-4,1′-inden]-1-yl]-N-[(2S)-1,1,1-trifluoropropan-2- |
| | yl]acetamide |
| 631 | N-benzyl-N-(cyanomethyl)-2-{(1R)-5-[(methylcarbamoyl)amino]-2′,4′- |
| | dioxo-2,3-dihydro-3′H-spiro[indene-1,5′-[1,3]ozazolidin]-3′-yl}acetamide |
| 632 | N-benzyl-N-(2-cyanoethyl)-2-{(1R)-5-[(methylcarbamoyl)amino]-2′,4′- |
| | dioxo-2,3-dihydro-3′H-spiro[indene-1,5′-[1,3]ozazolidin]-3′-yl}acetamide |
| 633 | N-(4-fluorobenzyl)-2-{(1R)-5-[(methylcarbamoyl)amino]-2′,4′-dioxo-2,3- |
| | dihydro-3′H-spiro[indene-1,5′-[1,3]ozazolidin]-3′-yl}-N-[1- |
| | (methylsulfonyl)propan-2-yl]acetamide |
| 634 | N-(4-fluorobenzyl)-2-{(1R)-5-[(methylcarbamoyl)amino]-2′,4′-dioxo-2,3- |
| | dihydro-3′H-spiro[indene-1,5′-[1,3]ozazolidin]-3′-yl}-N-[(1-methyl-1H- |
| | pyrazol-4-yl)methyl]acetamide |
| 635 | N-[(1S)-1-cyclopropylethyl]-N-(4-fluorobenzyl)-2-{4-methoxy-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 636 | 2-[5-(acetylamino)-4-methoxy-2′,4′-dioxo-2,3-dihydro-3′H-spiro[indene- |
| | 1,5′-[1,3]ozazolidin]-3′-yl]-N-[(1S)-1-cyclopropylethyl]-N-(4- |
| | fluorobenzyl)acetamide |
| 637 | N-(4-fluorobenzyl)-2-[(3′S,4R)-3′-fluoro-2,5-dioxo-2′,3′-dihydro-1H- |
| | spiro[imidazolidine-4,1′-inden]-1-yl]-N-[(2S)-1,1,1-trifluoropropan-2- |
| | yl]acetamide |
| 638 | 2-(7′-bromo-2,2′,5-trioxo-2′,3′-dihydro-1H,1′H-spiro[imidazolidine-4,4′- |
| | quinolin]-1-yl)-N-(4-fluorobenzyl)-N-[(2S)-1,1,1-trifluoropropan-2- |
| | yl]acetamide |
| 639 | 2-[(1R)-5-({[3-(acetylamino)-2,2-dimethylpropyl]carbamoyl}amino)-2′,4′- |
| | dioxo-2,3-dihydro-3′H-spiro[indene-1,5′-[1,3]ozazolidin]-3′-yl]-N-(4- |
| | fluorobenzyl)-N-[(2S)-1,1,1-trifluoropropan-2-yl]acetamide |
| 640 | N-(4-fluorobenzyl)-2-(7′-{1-[2-(methylamino)-2-oxoethyl]-1H-pyrazol-4- |
| | yl}-2,2′,5-trioxo-2′,3′-dihydro-1H,1′H-spiro[imidazolidine-4,4′-quinolin]- |
| | 1-yl)-N-[(2S)-1,1,1-trifluoropropan-2-yl]acetamide |
| 641 | 2-{7′-[1-(2-amino-2-oxoethyl)-1H-pyrazol-4-yl]-2,2′,5-trioxo-2′,3′- |
| | dihydro-1H,1′H-spiro[imidazolidine-4,4′-quinolin]-1-yl}-N-(4- |
| | fluorobenzyl)-N-[(2S)-1,1,1-trifluoropropan-2-yl]acetamide |
| 642 | N-cyclopentyl-2-{(1R)-5-[(methylcarbamoyl)amino]-2′,4′-dioxo-2,3- |
| | dihydro-3′H-spiro[indene-1,5′-[1,3]ozazolidin]-3′-yl}-N-[(1S)-1- |
| | phenylethyl]acetamide |
| 643 | N-[(1S)-1-cyclopropyl-2,2,2-trifluoroethyl]-N-(4-fluorobenzyl)-2-{1′- |
| | methyl-7′-[(methylcarbamoyl)amino]-2,5-dioxo-2′,3′-dihydro-1H,1′H- |
| | spiro[imidazolidine-4,4′-quinolin]-1-yl}acetamide |
| 644 | 2-(5′-amino-2,5-dioxo-2′,3′-dihydro-1H-spiro[imidazolidine-4,1′-inden]-1- |
| | yl)-N-(4-fluorobenzyl)-N-[(2S)-1,1,1-trifluoropropan-2-yl]acetamide |
| 645 | 2,2,2-trifluoro-N-[1-(2-{(4-fluorobenzyl)[(2S)-1,1,1-trifluoropropan-2- |
| | yl]amino}-2-oxoethyl)-1′-methyl-2,5-dioxo-2′,3′-dihydro-1′H- |
| | spiro[imidazolidine-4,4′-quinolin]-7′-yl]acetamide |
| 646 | N-[1-(2-{[(1S)-1-cyclopropylethyl](4-fluorobenzyl)amino}-2-oxoethyl)- |
| | 1′-methyl-2,5-dioxo-2′,3′-dihydro-1′H-spiro[imidazolidine-4,4′-quinolin]- |
| | 7′-yl]-2,2,2-trifluoroacetamide |
| 647 | N-(4-fluorobenzyl)-2-[(3′S,4S)-3′-hydroxy-2,5-dioxo-2′,3′-dihydro-1H- |
| | spiro[imidazolidine-4,1′-inden]-1-yl]-N-[(2S)-1,1,1-trifluoropropan-2- |
| | yl]acetamide |
| 648 | N-(4-fluorobenzyl)-2-{(1R)-5-[(methylcarbamoyl)amino]-2′,4′-dioxo-2,3- |
| | dihydro-3′H-spiro[indene-1,5′-[1,3]ozazolidin]-3′-yl}-N-(pyridin-3- |
| | yl)acetamide |
| 649 | N-(4-fluorobenzyl)-2-{(1R)-5-[(methylcarbamoyl)amino]-2′,4′-dioxo-2,3- |
| | dihydro-3′H-spiro[indene-1,5′-[1,3]ozazolidin]-3′-yl}-N-(1-methyl-1H- |
| | pyrazol-4-yl)acetamide |
| 650 | N-(4-fluorobenzyl)-2-[3′-fluoro-5′-(1-methyl-1H-pyrazol-4-yl)-2,5-dioxo- |
| | 2′,3′-dihydro-1H-spiro[imidazolidine-4,1′-inden]-1-yl]-N-[(2S)-1,1,1- |
| | trifluoropropan-2-yl]acetamide |
| 651 | N-benzyl-N-(2,2-difluorocyclopentyl)-2-{5-[(methylcarbamoyl)amino]- |
| | 2′,4′-dioxo-2,3-dihydro-3′H-spiro[indene-1,5′-[1,3]ozazolidin]-3′- |
| | yl}acetamide |
| 652 | N-(4-fluorobenzyl)-2-{5-[(methylcarbamoyl)amino]-2′,4′-dioxo-2,3- |
| | dihydro-3′H-spiro[indene-1,5′-[1,3]ozazolidin]-3′-yl}-N-(2- |
| | methylcyclopropyl)acetamide |
| 653 | N-(2,2-dimethylcyclopentyl)-N-(4-fluorobenzyl)-2-{5- |
| | [(methylcarbamoyl)amino]-2′,4′-dioxo-2,3-dihydro-3′H-spiro[indene-1,5′- |
| | [1,3]ozazolidin]-3′-yl}acetamide |
| 654 | N-(4-fluorobenzyl)-2-{5-[(methylcarbamoyl)amino]-2′,4′-dioxo-2,3- |
| | dihydro-3′H-spiro[indene-1,5′-[1,3]ozazolidin]-3′-yl}-N-(2- |
| | methylcyclopentyl)acetamide |
| 655 | 2-(5′-bromo-3′-hydroxy-2,5-dioxo-2′,3′-dihydro-1H-spiro[imidazolidine- |
| | 4,1′-inden]-1-yl)-N-(4-fluorobenzyl)-N-[(2S)-1,1,1-trifluoropropan-2- |
| | yl]acetamide |
| 656 | 2-(5-{[(2-cyanoethyl)carbamoyl]amino}-6-fluoro-2′,4′-dioxo-2,3-dihydro- |
| | 3′H-spiro[indene-1,5′-[1,3]ozazolidin]-3′-yl)-N-(4-fluorobenzyl)-N-[(2S)- |
| | 1,1,1-trifluoropropan-2-yl]acetamide |
| 657 | N-(4-fluorobenzyl)-2-[(3′R,4S)-3′-fluoro-2,5-dioxo-2′,3′-dihydro-1H- |
| | spiro[imidazolidine-4,1′-inden]-1-yl]-N-[(2S)-1,1,1-trifluoropropan-2- |
| | yl]acetamide |
| 658 | methyl [(1R)-3′-(2-{(4-fluorobenzyl)[(2S)-1,1,1-trifluoropropan-2- |
| | yl]amino}-2-oxoethyl)-2′,4′-dioxo-2,3-dihydrospiro[indene-1,5′- |
| | [1,3]ozazolidin]-5-yl]carbamate |
| 659 | 4-({[(1R)-3′-(2-{(4-fluorobenzyl)[(2S)-1,1,1-trifluoropropan-2-yl]amino}- |
| | 2-oxoethyl)-2′,4′-dioxo-2,3-dihydrospiro[indene-1,5′-[1,3]ozazolidin]-5- |
| | yl]carbamoyl}amino)butanoic acid |
| 660 | N²-acetyl-N-[(1R)-3′-(2-{(4-fluorobenzyl)[(2S)-1,1,1-trifluoropropan-2- |
| | yl]amino}-2-oxoethyl)-2′,4′-dioxo-2,3-dihydrospiro[indene-1,5′- |
| | [1,3]ozazolidin]-5-yl]-2-methylalaninamide |
| 661 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5-yl]- |
| | 2-methylalaninamide |
| 662 | N-cyclobutyl-2-{(1R)-5-[(methylcarbamoyl)amino]-2′,4′-dioxo-2,3- |
| | dihydro-3′H-spiro[indene-1,5′-[1,3]ozazolidin]-3′-yl}-N-[(1S)-1- |
| | phenylethyl]acetamide |
| 663 | N-cyclobutyl-2-{(1R)-5-[(methylcarbamoyl)amino]-2′,4′-dioxo-2,3- |
| | dihydro-3′H-spiro[indene-1,5′-[1,3]ozazolidin]-3′-yl}-N-[(1R)-1- |
| | phenylethyl]acetamide |
| 664 | N-cyclopentyl-2-{(1R)-5-[(methylcarbamoyl)amino]-2′,4′-dioxo-2,3- |
| | dihydro-3′H-spiro[indene-1,5′-[1,3]ozazolidin]-3′-yl}-N-[(1R)-1- |
| | phenylethyl]acetamide |
| 665 | N-(4-fluorobenzyl)-2-{(1R)-5-[(methylcarbamoyl)amino]-2′,4′-dioxo-2,3- |
| | dihydro-3′H-spiro[indene-1,5′-[1,3]ozazolidin]-3′-yl}-N-(1,3-oxazol-4- |
| | ylmethyl)acetamide |
| 666 | N-benzyl-2-{(1R)-5-[(methylcarbamoyl)amino]-2′,4′-dioxo-2,3-dihydro- |
| | 3′H-spiro[indene-1,5′-[1,3]ozazolidin]-3′-yl}-N-[1-(pyridin-2- |
| | yl)ethyl]acetamide |
| 667 | N-(4-fluorobenzyl)-2-{5′-[(methylcarbamoyl)amino]-2,3′,5-trioxo-2′,3′- |
| | dihydro-1H-spiro[imidazolidine-4,1′-inden]-1-yl}-N-[(2S)-1,1,1- |
| | trifluoropropan-2-yl]acetamide |
| 668 | N-(4-chlorobenzyl)-2-{5-[(methylcarbamoyl)amino]-2′,4′-dioxo-2,3- |
| | dihydro-3′H-spiro[indene-1,5′-[1,3]ozazolidin]-3′-yl}-N-[(2S)-1,1,1- |
| | trifluoropropan-2-yl]acetamide |
| 669 | N-(3,5-difluorobenzyl)-2-{5-[(methylcarbamoyl)amino]-2′,4′-dioxo-2,3- |
| | dihydro-3′H-spiro[indene-1,5′-[1,3]ozazolidin]-3′-yl}-N-[(2S)-1,1,1- |
| | trifluoropropan-2-yl]acetamide |
| 670 | 2-(5-{[(cyanomethyl)carbamoyl]amino}-6-fluoro-2′,4′-dioxo-2,3-dihydro- |
| | 3′H-spiro[indene-1,5′-[1,3]ozazolidin]-3′-yl)-N-(4-fluorobenzyl)-N-[(2S)- |
| | 1,1,1-trifluoropropan-2-yl]acetamide |
| 671 | 2-[(1R)-5-{[(cyanomethyl)carbamoyl]amino}-2′,4′-dioxo-2,3-dihydro-3′H- |
| | spiro[indene-1,5′-[1,3]ozazolidin]-3′-yl]-N-(4-fluorobenzyl)-N-[(2S)-1,1,1- |
| | trifluoropropan-2-yl]acetamide |
| 672 | N-cyclohexyl-2-{(1R)-5-[(methylcarbamoyl)amino]-2′,4′-dioxo-2,3- |
| | dihydro-3′H-spiro[indene-1,5′-[1,3]ozazolidin]-3′-yl}-N-[(1R)-1- |
| | phenylethyl]acetamide |
| 673 | 2-(5′-amino-2,3′,5-trioxo-2′,3′-dihydro-1H-spiro[imidazolidine-4,1′-inden]- |
| | 1-yl)-N-(4-fluorobenzyl)-N-[(2S)-1,1,1-trifluoropropan-2-yl]acetamide |
| 674 | tert-butyl (3R)-3-[(4-fluorobenzyl)({(1R)-5-[(methylcarbamoyl)amino]- |
| | 2′,4′-dioxo-2,3-dihydro-3′H-spiro[indene-1,5′-[1,3]ozazolidin]-3′- |
| | yl}acetyl)amino]pyrrolidine-1-carboxylate |
| 675 | N-(4-fluorobenzyl)-2-{(1R)-5-[(methylcarbamoyl)amino]-2′,4′-dioxo-2,3- |
| | dihydro-3′H-spiro[indene-1,5′-[1,3]ozazolidin]-3′-yl}-N-[(3R)-pyrrolidin- |
| | 3-yl]acetamide |
| 676 | N-(4-fluorobenzyl)-N-(trans-3-hydroxycyclobutyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 677 | N-(4-fluorobenzyl)-2-{(1R)-5-[(methylcarbamoyl)amino]-2′,4′-dioxo-2,3- |
| | dihydro-3′H-spiro[indene-1,5′-[1,3]ozazolidin]-3′-yl}-N-[(3R)-1- |
| | (methylsulfonyl)pyrrolidin-3-yl]acetamide |
| 678 | ethyl ({(3R)-3-[(4-fluorobenzyl)({(1R)-5-[(methylcarbamoyl)amino]-2′,4′- |
| | dioxo-2,3-dihydro-3′H-spiro[indene-1,5′-[1,3]ozazolidin]-3′- |
| | yl}acetyl)amino]pyrrolidin-1-yl}sulfonyl)carbamate |
| 679 | N-(4-fluorobenzyl)-2-[6-(1-methyl-1H-pyrazol-4-yl)-1,1-dioxido-2′,5′- |
| | dioxo-1′H-spiro[1-benzothiophene-3,4′-imidazolidin]-1′-yl]-N-[(2S)-1,1,1- |
| | trifluoropropan-2-yl]acetamide |
| 680 | 2-[(4S)-5′-bromo-2,5-dioxo-2′,3′-dihydro-1H-spiro[imidazolidine-4,1′- |
| | inden]-1-yl]-N-(4-fluorobenzyl)-N-[(2S)-1,1,1-trifluoropropan-2- |
| | yl]acetamide |
| 681 | N-(4-fluorobenzyl)-2-{3′-hydroxy-5′-[1-(2-methylpropyl)-1H-pyrazol-4- |
| | yl]-2,5-dioxo-2′,3′-dihydro-1H-spiro[imidazolidine-4,1′-inden]-1-yl}-N- |
| | [(2S)-1,1,1-trifluoropropan-2-yl]acetamide |
| 682 | N-(4-fluorobenzyl)-2-[(4S)-5′-(1-methyl-1H-pyrazol-4-yl)-2,5-dioxo-2′,3′- |
| | dihydro-1H-spiro[imidazolidine-4,1′-inden]-1-yl]-N-[(2S)-1,1,1- |
| | trifluoropropan-2-yl]acetamide |
| 683 | N-(4-fluorobenzyl)-2-[(4S)-5′-{1-[2-(methylamino)-2-oxoethyl]-1H- |
| | pyrazol-4-yl}-2,5-dioxo-2′,3′-dihydro-1H-spiro[imidazolidine-4,1′-inden]- |
| | 1-yl]-N-[(2S)-1,1,1-trifluoropropan-2-yl]acetamide |
| 684 | N-(1-cyanopropan-2-yl)-N-(4-fluorobenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 685 | 2-[(4S)-5′-bromo-2,3′,5-trioxo-2′,3′-dihydro-1H-spiro[imidazolidine-4,1′- |
| | inden]-1-yl]-N-(4-fluorobenzyl)-N-[(2S)-1,1,1-trifluoropropan-2- |
| | yl]acetamide |
| 686 | 2-(5′-bromo-3′-fluoro-2,5-dioxo-2′,3′-dihydro-1H-spiro[imidazolidine-4,1′- |
| | inden]-1-yl)-N-(4-fluorobenzyl)-N-[(2S)-1,1,1-trifluoropropan-2- |
| | yl]acetamide |
| 687 | 5-({[(1R)-3′-(2-{(4-fluorobenzyl)[(2S)-1,1,1-trifluoropropan-2-yl]amino}- |
| | 2-oxoethyl)-2′,4′-dioxo-2,3-dihydrospiro[indene-1,5′-[1,3]ozazolidin]-5- |
| | yl]carbamoyl}amino)pentanoic acid |
| 688 | N-[(1S)-1-cyclopropyl-2,2,2-trifluoroethyl]-N-(4-fluorobenzyl)-2-[4- |
| | fluoro-5-(1-methyl-1H-pyrazol-4-yl)-2′,4′-dioxo-2,3-dihydro-3′H- |
| | spiro[indene-1,5′-[1,3]ozazolidin]-3′-yl]acetamide |
| 689 | N-(4-fluorobenzyl)-2-[6-fluoro-5-(1-methyl-1H-pyrazol-4-yl)-2′,4′-dioxo- |
| | 2,3-dihydro-3′H-spiro[indene-1,5′-[1,3]ozazolidin]-3′-yl]-N-[(2S)-1,1,1- |
| | trifluoropropan-2-yl]acetamide |
| 690 | 2-{(4S)-5′-[1-(2-amino-2-oxoethyl)-1H-pyrazol-4-yl]-2,5-dioxo-2′,3′- |
| | dihydro-1H-spiro[imidazolidine-4,1′-inden]-1-yl}-N-(4-fluorobenzyl)-N- |
| | [(2S)-1,1,1-trifluoropropan-2-yl]acetamide |
| 691 | N-[(1S)-1-cyclopropylethyl]-N-(4-fluorobenzyl)-2-[1′-methyl-7′-(1- |
| | methyl-1H-pyrazol-4-yl)-2,5-dioxo-2′,3′-dihydro-1H,1′H- |
| | spiro[imidazolidine-4,4′-quinolin]-1-yl]acetamide |
| 692 | N-(4-fluorobenzyl)-2-[7′-(1-methyl-1H-pyrazol-4-yl)-2,2′,5-trioxo-2′,3′- |
| | dihydro-1H,1′H-spiro[imidazolidine-4,4′-quinolin]-1-yl]-N-[(2S)-1,1,1- |
| | trifluoropropan-2-yl]acetamide |
| 693 | N-(4-fluorobenzyl)-2-[(4S)-5′-(1-methyl-1H-pyrazol-4-yl)-2,3′,5-trioxo- |
| | 2′,3′-dihydro-1H-spiro[imidazolidine-4,1′-inden]-1-yl]-N-[(2S)-1,1,1- |
| | trifluoropropan-2-yl]acetamide |
| 694 | N-[(3R)-1-acetylpyrrolidin-3-yl]-N-(4-fluorobenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 695 | N-(4-fluorobenzyl)-2-{(1R)-5-[(methylcarbamoyl)amino]-2′,4′-dioxo-2,3- |
| | dihydro-3′H-spiro[indene-1,5′-[1,3]ozazolidin]-3′-yl}-N-[(3R)-1-(2- |
| | sulfamoylethyl)pyrrolidin-3-yl]acetamide |
| 696 | N-(4-fluorobenzyl)-N-(trans-4-hydroxycyclohexyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 697 | methyl (1R,3S)-3-[(4-fluorobenzyl)({(1R)-5-[(methylcarbamoyl)amino]- |
| | 2′,4′-dioxo-2,3-dihydro-3′H-spiro[indene-1,5′-[1,3]ozazolidin]-3′- |
| | yl}acetyl)amino]cyclohexanecarboxylate |
| 698 | N-(4-fluorobenzyl)-2-[(3′R,4S)-3′-hydroxy-2,5-dioxo-2′,3′-dihydro-1H- |
| | spiro[imidazolidine-4,1′-inden]-1-yl]-N-[(2S)-1,1,1-trifluoropropan-2- |
| | yl]acetamide |
| 699 | N-(4-fluorobenzyl)-2-[(3′S,4S)-3′-hydroxy-5′-(1-methyl-1H-pyrazol-4-yl)- |
| | 2,5-dioxo-2′,3′-dihydro-1H-spiro[imidazolidine-4,1′-inden]-1-yl]-N-[(2S)- |
| | 1,1,1-trifluoropropan-2-yl]acetamide |
| 700 | N-(4-fluorobenzyl)-2-[(3′R,4S)-3′-fluoro-5′-(1-methyl-1H-pyrazol-4-yl)- |
| | 2,5-dioxo-2′,3′-dihydro-1H-spiro[imidazolidine-4,1′-inden]-1-yl]-N-[(2S)- |
| | 1,1,1-trifluoropropan-2-yl]acetamide |
| 701 | N-(4-fluorobenzyl)-2-[(4S)-5′-{1-[2-(methylamino)-2-oxoethyl]-1H- |
| | pyrazol-4-yl}-2,3′,5-trioxo-2′,3′-dihydro-1H-spiro[imidazolidine-4,1′- |
| | inden]-1-yl]-N-[(2S)-1,1,1-trifluoropropan-2-yl]acetamide |
| 702 | N-[(1R)-3′-(2-{(4-fluorobenzyl)[(2S)-1,1,1-trifluoropropan-2-yl]amino}-2- |
| | oxoethyl)-2′,4′-dioxo-2,3-dihydrospiro[indene-1,5′-[1,3]ozazolidin]-5-yl]- |
| | 3-(methylsulfonyl)tetrahydropyrimidine-1(2H)-carboxamide |
| 703 | N-(4-fluorobenzyl)-2-[(3′S,4S)-3′-hydroxy-5′-{1-[2-(methylamino)-2- |
| | oxoethyl]-1H-pyrazol-4-yl}-2,5-dioxo-2′,3′-dihydro-1H- |
| | spiro[imidazolidine-4,1′-inden]-1-yl]-N-[(2S)-1,1,1-trifluoropropan-2- |
| | yl]acetamide |
| 704 | N-(3,5-difluorobenzyl)-2-[(4S)-5′-{1-[2-(methylamino)-2-oxoethyl]-1H- |
| | pyrazol-4-yl}-2,3′,5-trioxo-2′,3′-dihydro-1H-spiro[imidazolidine-4,1′- |
| | inden]-1-yl]-N-[(2S)-1,1,1-trifluoropropan-2-yl]acetamide |
| 705 | N-(3,5-difluorobenzyl)-2-[(3′S,4S)-3′-hydroxy-5′-{1-[2-(methylamino)-2- |
| | oxoethyl]-1H-pyrazol-4-yl}-2,5-dioxo-2′,3′-dihydro-1H- |
| | spiro[imidazolidine-4,1′-inden]-1-yl]-N-[(2S)-1,1,1-trifluoropropan-2- |
| | yl]acetamide |
| 706 | N-(3,5-difluorobenzyl)-2-[(3′R,4S)-3′-fluoro-5′-{1-[2-(methylamino)-2- |
| | oxoethyl]-1H-pyrazol-4-yl}-2,5-dioxo-2′,3′-dihydro-1H- |
| | spiro[imidazolidine-4,1′-inden]-1-yl]-N-[(2S)-1,1,1-trifluoropropan-2- |
| | yl]acetamide |
| 707 | N²-acetyl-N-[(1R)-3′-(2-{(4-fluorobenzyl)[(2S)-1,1,1-trifluoropropan-2- |
| | yl]amino}-2-oxoethyl)-2′,4′-dioxo-2,3-dihydrospiro[indene-1,5′- |
| | [1,3]ozazolidin]-5-yl]glycinamide |
| 708 | 2-{(1R)-5-[(methylcarbamoyl)amino]-2′,4′-dioxo-2,3-dihydro-3′H- |
| | spiro[indene-1,5′-[1,3]ozazolidin]-3′-yl}-N-[1-(2-methylphenyl)ethyl]-N- |
| | (2-methylpropyl)acetamide |
| 709 | N-(2-chlorobenzyl)-N-(cyclopropylmethyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 710 | N-(2-fluorobenzyl)-2-{(1R)-5-[(methylcarbamoyl)amino]-2′,4′-dioxo-2,3- |
| | dihydro-3′H-spiro[indene-1,5′-[1,3]ozazolidin]-3′-yl}-N-(2- |
| | methylpropyl)acetamide |
| 711 | 2-[(3′S,4S)-5′-bromo-3′-hydroxy-2,5-dioxo-2′,3′-dihydro-1H- |
| | spiro[imidazolidine-4,1′-inden]-1-yl]-N-(4-fluorobenzyl)-N-[(2S)-1,1,1- |
| | trifluoropropan-2-yl]acetamide |
| 712 | N-(4-fluorobenzyl)-N-(4-methoxyphenyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 713 | N-(4-fluorobenzyl)-N-(4-fluorophenyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 714 | N-(4-fluorobenzyl)-N-(2-fluorophenyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 715 | N-(4-fluorobenzyl)-2-[(3′R,4S)-3′-fluoro-5′-{1-[2-(methylamino)-2- |
| | oxoethyl]-1H-pyrazol-4-yl}-2,5-dioxo-2′,3′-dihydro-1H- |
| | spiro[imidazolidine-4,1′-inden]-1-yl]-N-[(2S)-1,1,1-trifluoropropan-2- |
| | yl]acetamide |
| 716 | 2-[(3′R,4S)-5′-bromo-3′-hydroxy-2,5-dioxo-2′,3′-dihydro-1H- |
| | spiro[imidazolidine-4,1′-inden]-1-yl]-N-(4-fluorobenzyl)-N-[(2S)-1,1,1- |
| | trifluoropropan-2-yl]acetamide |
| 717 | 2-{(4S)-5′-[1-(2-amino-2-oxoethyl)-1H-pyrazol-4-yl]-2,3′,5-trioxo-2′,3′- |
| | dihydro-1H-spiro[imidazolidine-4,1′-inden]-1-yl}-N-(4-fluorobenzyl)-N- |
| | [(2S)-1,1,1-trifluoropropan-2-yl]acetamide |
| 718 | N-(4-fluorobenzyl)-2-{(1R)-5-[(methylcarbamoyl)amino]-2′,4′-dioxo-2,3- |
| | dihydro-3′H-spiro[indene-1,5′-[1,3]ozazolidin]-3′-yl}-N-[1- |
| | (trifluoromethyl)cyclopropyl]acetamide |
| 719 | 2-[(1R)-5-(3,4-dihydro-2H-pyrrol-5-ylamino)-2′,4′-dioxo-2,3-dihydro-3′H- |
| | spiro[indene-1,5′-[1,3]ozazolidin]-3′-yl]-N-(4-fluorobenzyl)-N-[(2S)-1,1,1- |
| | trifluoropropan-2-yl]acetamide |
| 720 | N-(3,4-difluorobenzyl)-2-{5-[(methylcarbamoyl)amino]-2′,4′-dioxo-2,3- |
| | dihydro-3′H-spiro[indene-1,5′-[1,3]ozazolidin]-3′-yl}-N-[(2S)-1,1,1- |
| | trifluoropropan-2-yl]acetamide |
| 721 | N-(3-fluorobenzyl)-2-{5-[(methylcarbamoyl)amino]-2′,4′-dioxo-2,3- |
| | dihydro-3′H-spiro[indene-1,5′-[1,3]ozazolidin]-3′-yl}-N-[(2S)-1,1,1- |
| | trifluoropropan-2-yl]acetamide |
| 722 | N-(2,5-difluorobenzyl)-2-{5-[(methylcarbamoyl)amino]-2′,4′-dioxo-2,3- |
| | dihydro-3′H-spiro[indene-1,5′-[1,3]ozazolidin]-3′-yl}-N-[(2S)-1,1,1- |
| | trifluoropropan-2-yl]acetamide |
| 723 | N-(3-chlorobenzyl)-2-{5-[(methylcarbamoyl)amino]-2′,4′-dioxo-2,3- |
| | dihydro-3′H-spiro[indene-1,5′-[1,3]ozazolidin]-3′-yl}-N-[(2S)-1,1,1- |
| | trifluoropropan-2-yl]acetamide |
| 724 | 2-{(3′S,4S)-5′-[1-(2-amino-2-oxoethyl)-1H-pyrazol-4-yl]-3′-hydroxy-2,5- |
| | dioxo-2′,3′-dihydro-1H-spiro[imidazolidine-4,1′-inden]-1-yl}-N-(4- |
| | fluorobenzyl)-N-[(2S)-1,1,1-trifluoropropan-2-yl]acetamide |
| 725 | 2-{5-[1-(2-amino-2-oxoethyl)-1H-pyrazol-4-yl]-6-fluoro-2′,4′-dioxo-2,3- |
| | dihydro-3′H-spiro[indene-1,5′-[1,3]ozazolidin]-3′-yl}-N-(4-fluorobenzyl)- |
| | N-[(2S)-1,1,1-trifluoropropan-2-yl]acetamide |
| 726 | tert-butyl 4-[(4-fluorobenzyl)({(1R)-5-[(methylcarbamoyl)amino]-2′,4′- |
| | dioxo-2,3-dihydro-3′H-spiro[indene-1,5′-[1,3]ozazolidin]-3′- |
| | yl}acetyl)amino]piperidine-1-carboxylate |
| 727 | 2-[(1R)-5-bromo-2′,4′-dioxo-2,3-dihydro-3′H-spiro[indene-1,5′- |
| | [1,3]ozazolidin]-3′-yl]-N-(4-fluorobenzyl)-N-[(3R)-pyrrolidin-3- |
| | yl]acetamide |
| 728 | ethyl ({(3R)-3-[{[(1R)-5-bromo-2′,4′-dioxo-2,3-dihydro-3′H-spiro[indene- |
| | 1,5′-[1,3]ozazolidin]-3′-yl]acetyl}(4-fluorobenzyl)amino]pyrrolidin-1- |
| | yl}sulfonyl)carbamate |
| 729 | N-(4-fluorobenzyl)-2-{(1R)-5-[(methylcarbamoyl)amino]-2′,4′-dioxo-2,3- |
| | dihydro-3′H-spiro[indene-1,5′-[1,3]ozazolidin]-3′-yl}-N-(piperidin-4- |
| | yl)acetamide |
| 730 | ethyl ({4-[(4-fluorobenzyl)({(1R)-5-[(methylcarbamoyl)amino]-2′,4′- |
| | dioxo-2,3-dihydro-3′H-spiro[indene-1,5′-[1,3]ozazolidin]-3′- |
| | yl}acetyl)amino]piperidin-1-yl}sulfonyl)carbamate |
| 731 | 2-[(3′S,4S)-5′-{1-[2-(dimethylamino)-2-oxoethyl]-1H-pyrazol-4-yl}-3′- |
| | hydroxy-2,5-dioxo-2′,3′-dihydro-1H-spiro[imidazolidine-4,1′-inden]-1-yl]- |
| | N-(4-fluorobenzyl)-N-[(2S)-1,1,1-trifluoropropan-2-yl]acetamide |
| 732 | tert-butyl 3-[(4-fluorobenzyl)({(1R)-5-[(methylcarbamoyl)amino]-2′,4′- |
| | dioxo-2,3-dihydro-3′H-spiro[indene-1,5′-[1,3]ozazolidin]-3′- |
| | yl}acetyl)amino]azetidine-1-carboxylate |
| 733 | N-(cyclopropylmethyl)-2-{(1R)-5-[(methylcarbamoyl)amino]-2′,4′-dioxo- |
| | 2,3-dihydro-3′H-spiro[indene-1,5′-[1,3]ozazolidin]-3′-yl}-N-[2- |
| | (trifluoromethyl)benzyl]acetamide |
| 734 | N-(2-chloro-4-fluorobenzyl)-N-(cyclopropylmethyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 735 | 2-[(3′R,4S)-5′-{1-[2-(dimethylamino)-2-oxoethyl]-1H-pyrazol-4-yl}-3′- |
| | fluoro-2,5-dioxo-2′,3′-dihydro-1H-spiro[imidazolidine-4,1′-inden]-1-yl]-N- |
| | (4-fluorobenzyl)-N-[(2S)-1,1,1-trifluoropropan-2-yl]acetamide |
| 736 | N-(4-fluorobenzyl)-N-(3-methoxyphenyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 737 | N-benzyl-2-{(1R)-5-[(methylcarbamoyl)amino]-2′,4′-dioxo-2,3-dihydro- |
| | 3′H-spiro[indene-1,5′-[1,3]ozazolidin]-3′-yl}-N-(pyridin-4-yl)acetamide |
| 738 | N-(4-fluorobenzyl)-2-[(4S)-5′-{1-[2-(methylamino)-2-oxoethyl]-1H- |
| | pyrazol-4-yl}-2,5-dioxo-1H-spiro[imidazolidine-4,1′-inden]-1-yl]-N-[(2S)- |
| | 1,1,1-trifluoropropan-2-yl]acetamide |
| 739 | N-(3,4-difluorobenzyl)-2-[(4S)-5′-(1-methyl-1H-pyrazol-4-yl)-2,5-dioxo- |
| | 2′,3′-dihydro-1H-spiro[imidazolidine-4,1′-inden]-1-yl]-N-[(2S)-1,1,1- |
| | trifluoropropan-2-yl]acetamide |
| 740 | N-(4-chlorobenzyl)-2-[(4S)-5′-(1-methyl-1H-pyrazol-4-yl)-2,5-dioxo-2′,3′- |
| | dihydro-1H-spiro[imidazolidine-4,1′-inden]-1-yl]-N-[(2S)-1,1,1- |
| | trifluoropropan-2-yl]acetamide |
| 741 | N-(3,5-difluorobenzyl)-2-[(4S)-5′-(1-methyl-1H-pyrazol-4-yl)-2,5-dioxo- |
| | 2′,3′-dihydro-1H-spiro[imidazolidine-4,1′-inden]-1-yl]-N-[(2S)-1,1,1- |
| | trifluoropropan-2-yl]acetamide |
| 742 | N-(3-fluorobenzyl)-2-[(4S)-5′-(1-methyl-1H-pyrazol-4-yl)-2,5-dioxo-2′,3′- |
| | dihydro-1H-spiro[imidazolidine-4,1′-inden]-1-yl]-N-[(2S)-1,1,1- |
| | trifluoropropan-2-yl]acetamide |
| 743 | N-(2,5-difluorobenzyl)-2-[(4S)-5′-(1-methyl-1H-pyrazol-4-yl)-2,5-dioxo- |
| | 2′,3′-dihydro-1H-spiro[imidazolidine-4,1′-inden]-1-yl]-N-[(2S)-1,1,1- |
| | trifluoropropan-2-yl]acetamide |
| 744 | N-(3-chlorobenzyl)-2-[(4S)-5′-(1-methyl-1H-pyrazol-4-yl)-2,5-dioxo-2′,3′- |
| | dihydro-1H-spiro[imidazolidine-4,1′-inden]-1-yl]-N-[(2S)-1,1,1- |
| | trifluoropropan-2-yl]acetamide |
| 745 | N-[(1S)-1-cyclopropylethyl]-N-(4-fluorobenzyl)-2-[(4S)-5′-(1-methyl-1H- |
| | pyrazol-4-yl)-2,5-dioxo-2′,3′-dihydro-1H-spiro[imidazolidine-4,1′-inden]- |
| | 1-yl]acetamide |
| 746 | N-[(1S)-1-cyclopropyl-2,2,2-trifluoroethyl]-N-(4-fluorobenzyl)-2-[(4S)-5′- |
| | (1-methyl-1H-pyrazol-4-yl)-2,5-dioxo-2′,3′-dihydro-1H- |
| | spiro[imidazolidine-4,1′-inden]-1-yl]acetamide |
| 747 | N-[(1R)-1-cyclopropylethyl]-N-(4-fluorobenzyl)-2-[(4S)-5′-(1-methyl-1H- |
| | pyrazol-4-yl)-2,5-dioxo-2′,3′-dihydro-1H-spiro[imidazolidine-4,1′-inden]- |
| | 1-yl]acetamide |
| 748 | N-benzyl-N-[(1S)-1-cyclopropylethyl]-2-[(4S)-5′-(1-methyl-1H-pyrazol-4- |
| | yl)-2,5-dioxo-2′,3′-dihydro-1H-spiro[imidazolidine-4,1′-inden]-1- |
| | yl]acetamide |
| 749 | N-benzyl-N-[(1R)-1-cyclopropylethyl]-2-[(4S)-5′-(1-methyl-1H-pyrazol- |
| | 4-yl)-2,5-dioxo-2′,3′-dihydro-1H-spiro[imidazolidine-4,1′-inden]-1- |
| | yl]acetamide |
| 750 | 2-(4′-bromo-2,5-dioxo-2′,3′-dihydro-1H-spiro[imidazolidine-4,1′-inden]-1- |
| | yl)-N-(4-fluorobenzyl)-N-[(2S)-1,1,1-trifluoropropan-2-yl]acetamide |
| 751 | N-(4-fluorobenzyl)-2-[4′-(1-methyl-1H-pyrazol-4-yl)-2,5-dioxo-2′,3′- |
| | dihydro-1H-spiro[imidazolidine-4,1′-inden]-1-yl]-N-[(2S)-1,1,1- |
| | trifluoropropan-2-yl]acetamide |
| 752 | N-(4-fluorobenzyl)-2-(6-fluoro-5-{1-[2-(methylamino)-2-oxoethyl]-1H- |
| | pyrazol-4-yl}-2′,4′-dioxo-2,3-dihydro-3′H-spiro[indene-1,5′- |
| | [1,3]ozazolidin]-3′-yl)-N-[(2S)-1,1,1-trifluoropropan-2-yl]acetamide |
| 753 | 2-[(3′S,4S)-5′-{1-[2-(dimethylamino)-2-oxoethyl]-1H-pyrazol-4-yl}-3′- |
| | fluoro-2,5-dioxo-2′,3′-dihydro-1H-spiro[imidazolidine-4,1′-inden]-1-yl]-N- |
| | (4-fluorobenzyl)-N-[(2S)-1,1,1-trifluoropropan-2-yl]acetamide |
| 754 | methyl (1S,3S)-3-[(4-fluorobenzyl)({(1R)-5-[(methylcarbamoyl)amino]- |
| | 2′,4′-dioxo-2,3-dihydro-3′H-spiro[indene-1,5′-[1,3]ozazolidin]-3′- |
| | yl}acetyl)amino]cyclohexanecarboxylate |
| 755 | N-(4-fluorobenzyl)-2-{(1R)-5-[(methylcarbamoyl)amino]-2′,4′-dioxo-2,3- |
| | dihydro-3′H-spiro[indene-1,5′-[1,3]ozazolidin]-3′-yl}-N-(tetrahydro-2H- |
| | pyran-4-yl)acetamide |
| 756 | N-(1,1-dioxidotetrahydro-2H-thiopyran-4-yl)-N-(4-fluorobenzyl)-2-{(1R)- |
| | 5-[(methylcarbamoyl)amino]-2′,4′-dioxo-2,3-dihydro-3′H-spiro[indene- |
| | 1,5′-[1,3]ozazolidin]-3′-yl}acetamide |
| 757 | N-(2,2-difluorocyclopentyl)-N-(4-fluorobenzyl)-2-{(1R)-5- |
| | [(methylcarbamoyl)amino]-2′,4′-dioxo-2,3-dihydro-3′H-spiro[indene-1,5′- |
| | [1,3]ozazolidin]-3′-yl}acetamide |
| 758 | N-cyclohexyl-2-{(1R)-5-[(methylcarbamoyl)amino]-2′,4′-dioxo-2,3- |
| | dihydro-3′H-spiro[indene-1,5′-[1,3]ozazolidin]-3′-yl}-N-[(1S)-1- |
| | phenylethyl]acetamide |
| 759 | 2-[(3S)-6-amino-1,1-dioxido-2′,5′-dioxo-1′H-spiro[1-benzothiophene- |
| | 3,4′-imidazolidin]-1′-yl]-N-(4-fluorobenzyl)-N-[(2S)-1,1,1- |
| | trifluoropropan-2-yl]acetamide |
| 760 | 2-[(3′R,4S)-5′-bromo-3′-fluoro-2,5-dioxo-2′,3′-dihydro-1H- |
| | spiro[imidazolidine-4,1′-inden]-1-yl]-N-(4-fluorobenzyl)-N-[(2S)-1,1,1- |
| | trifluoropropan-2-yl]acetamide |
| 761 | 2-(5′-bromo-6′-fluoro-2,3′,5-trioxo-2′,3′-dihydro-1H-spiro[imidazolidine-4,1′- |
| | inden]-1-yl)-N-(4-fluorobenzyl)-N-[(2S)-1,1,1-trifluoropropan-2-yl]acetamide |
| 762 | 2-(5′-bromo-6′-fluoro-2,5-dioxo-2′,3′-dihydro-1H-spiro[imidazolidine-4,1′- |
| | inden]-1-yl)-N-(4-fluorobenzyl)-N-[(2S)-1,1,1-trifluoropropan-2-yl]acetamide |
| 763 | N-(4-fluorobenzyl)-2-(6′-fluoro-5′-{1-[2-(methylamino)-2-oxoethyl]-1H- |
| | pyrazol-4-yl}-2,3′,5-trioxo-2′,3′-dihydro-1H-spiro[imidazolidine-4,1′-inden]-1- |
| | yl)-N-[(2S)-1,1,1-trifluoropropan-2-yl]acetamide |
| 764 | N-(4-fluorobenzyl)-2-(6′-fluoro-3′-hydroxy-5′-{1-[2-(methylamino)-2- |
| | oxoethyl]-1H-pyrazol-4-yl}-2,5-dioxo-2′,3′-dihydro-1H-spiro[imidazolidine- |
| | 4,1′-inden]-1-yl)-N-[(2S)-1,1,1-trifluoropropan-2-yl]acetamide |
| 765 | N-(4-fluorobenzyl)-2-[4′-(1-methyl-1,2,3,6-tetrahydropyridin-4-yl)-2,5-dioxo- |
| | 2′,3′-dihydro-1H-spiro[imidazolidine-4,1′-inden]-1-yl]-N-[(2S)-1,1,1- |
| | trifluoropropan-2-yl]acetamide |
| 766 | 2-(3′,6′-difluoro-5′-{1-[2-(methylamino)-2-oxoethyl]-1H-pyrazol-4-yl}-2,5- |
| | dioxo-2′,3′-dihydro-1H-spiro[imidazolidine-4,1′-inden]-1-yl)-N-(4- |
| | fluorobenzyl)-N-[(2S)-1,1,1-trifluoropropan-2-yl]acetamide |
| 767 | N-[(1S)-1-cyclopropyl-2,2,2-trifluoroethyl]-N-(4-fluorobenzyl)-2-[7-(1- |
| | methyl-1H-pyrazol-4-yl)-2′,4′-dioxo-1H,3′H-spiro[isochromene-4,5′- |
| | [1,3]oxazolidin]-3′-yl]acetamide |
| 768 | 2-(7-amino-2′,4′-dioxo-1H,3′H-spiro[isochromene-4,5′-[1,3]oxazolidin]-3′-yl)- |
| | N-[(1S)-1-cyclopropyl-2,2,2-trifluoroethyl]-N-(4-fluorobenzyl)acetamide |
| 769 | N-[(1S)-1-cyclopropyl-2,2,2-trifluoroethyl]-N-(4-fluorobenzyl)-2-{7- |
| | [(methylcarbamoyl)amino]-2′,4′-dioxo-1H,3′H-spiro[isochromene-4,5′- |
| | [1,3]oxazolidin]-3′-yl}acetamide |
| 770 | N-(4-fluorobenzyl)-2-{7-[(methylcarbamoyl)amino]-2′,4′-dioxo-1H,3′H- |
| | spiro[isochromene-4,5′-[1,3]oxazolidin]-3′-yl}-N-[(2S)-1,1,1-trifluoropropan- |
| | 2-yl]acetamide |
| 771 | N-(4-fluorobenzyl)-2-[7-(1-methyl-1H-pyrazol-4-yl)-2′,4′-dioxo-1H,3′H- |
| | spiro[isochromene-4,5′-[1,3]oxazolidin]-3′-yl]-N-[(2S)-1,1,1-trifluoropropan- |
| | 2-yl]acetamide |
| 772 | N-(4-fluorobenzyl)-2-(7-{1-[2-(methylamino)-2-oxoethyl]-1H-pyrazol-4-yl}- |
| | 2′,4′-dioxo-1H,3′H-spiro[isochromene-4,5′-[1,3]oxazolidin]-3′-yl)-N-[(2S)- |
| | 1,1,1-trifluoropropan-2-yl]acetamide |
| 773 | N-(4-fluorobenzyl)-2-[(3′R,4S)-3′-hydroxy-5′-{1-[2-(methylamino)-2- |
| | oxoethyl]-1H-pyrazol-4-yl}-2,5-dioxo-2′,3′-dihydro-1H-spiro[imidazolidine- |
| | 4,1′-inden]-1-yl]-N-[(2S)-1,1,1-trifluoropropan-2-yl]acetamide |
| 774 | 2-[(3R)-6-amino-1,1-dioxido-2′,5′-dioxo-1′H-spiro[1-benzothiophene-3,4′- |
| | imidazolidin]-1′-yl]-N-(4-fluorobenzyl)-N-[(2S)-1,1,1-trifluoropropan-2- |
| | yl]acetamide |
| 775 | N-(4-fluorobenzyl)-N-{1-[2-(methylamino)-2-oxoethyl]-1H-pyrazol-4-yl}-2- |
| | {(1R)-5-[(methylcarbamoyl)amino]-2′,4′-dioxo-2,3-dihydro-3′H-spiro[indene- |
| | 1,5′-[1,3]oxazolidin]-3′-yl}acetamide |
| 776 | N-[1-(cyanomethyl)-1H-pyrazol-4-yl]-N-(4-fluorobenzyl)-2-{(1R)-5- |
| | [(methylcarbamoyl)amino]-2′,4′-dioxo-2,3-dihydro-3′H-spiro[indene-1,5′- |
| | [1,3]oxazolidin]-3′-yl}acetamide |
| 777 | tert-butyl 3-{1-[(4-fluorobenzyl)({(1R)-5-[(methylcarbamoyl)amino]-2′,4′- |
| | dioxo-2,3-dihydro-3′H-spiro[indene-1,5′-[1,3]oxazolidin]-3′- |
| | yl}acetyl)amino]ethyl}azetidine-1-carboxylate |
| 778 | tert-butyl 4-[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′- |
| | yl}acetyl)amino]cyclohexanecarboxylate |
| 779 | 4-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′- |
| | yl}acetyl)amino]cyclohexanecarboxylic acid |
| 780 | ethyl ({(3S)-3-[(4-fluorobenzyl){[(1R)-5-{1-[2-(methylamino)-2-oxoethyl]- |
| | 1H-pyrazol-4-yl}-2′,4′-dioxo-2,3-dihydro-3′H-spiro[indene-1,5′- |
| | [1,3]oxazolidin]-3′-yl]acetyl}amino]pyrrolidin-1-yl}sulfonyl)carbamate |
| 781 | ethyl ({(3S)-3-[{[(1R)-5-{1-[2-(dimethylamino)-2-oxoethyl]-1H-pyrazol-4- |
| | yl}-2′,4′-dioxo-2,3-dihydro-3′H-spiro[indene-1,5′-[1,3]oxazolidin]-3′- |
| | yl]acetyl}(4-fluorobenzyl)amino]pyrrolidin-1-yl}sulfonyl)carbamate |
| 782 | tert-butyl {4-[(4-fluorobenzyl)({(1R)-5-[(methylcarbamoyl)amino]-2′,4′- |
| | dioxo-2,3-dihydro-3′H-spiro[indene-1,5′-[1,3]oxazolidin]-3′- |
| | yl}acetyl)amino]piperidin-1-yl}acetate |
| 783 | ethyl ({(3R)-3-[(4-fluorobenzyl){[(1R)-5-{1-[2-(methylamino)-2-oxoethyl]- |
| | 1H-pyrazol-4-yl}-2′,4′-dioxo-2,3-dihydro-3′H-spiro[indene-1,5′- |
| | [1,3]oxazolidin]-3′-yl]acetyl}amino]piperidin-1-yl}sulfonyl)carbamate |
| 784 | ethyl ({(3R)-3-[(4-fluorobenzyl){[(1R)-5-(1-methyl-1H-pyrazol-4-yl)-2′,4′- |
| | dioxo-2,3-dihydro-3′H-spiro[indene-1,5′-[1,3]oxazolidin]-3′- |
| | yl]acetyl}amino]piperidin-1-yl}sulfonyl)carbamate |
| 785 | {4-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′-yl}acetyl)amino]piperidin-1- |
| | yl}acetic acid |
| 786 | 2-{2,5-dioxo-5′-[(2,2,2-trifluoroethyl)amino]-2′,3′-dihydro-1H- |
| | spiro[imidazolidine-4,1′-inden]-1-yl}-N-(4-fluorobenzyl)-N-[(2S)-1,1,1- |
| | trifluoropropan-2-yl]acetamide |
| 787 | 2-[(4S)-5′-(acetylamino)-2,5-dioxo-2′,3′-dihydro-1H-spiro[imidazolidine-4,1′- |
| | inden]-1-yl]-N-(4-fluorobenzyl)-N-[(2S)-1,1,1-trifluoropropan-2-yl]acetamide |
| 788 | 2-[(4R)-5′-bromo-2,5-dioxo-2′,3′-dihydro-1H-spiro[imidazolidine-4,1′-inden]- |
| | 1-yl]-N-(4-fluorobenzyl)-N-[(2S)-1,1,1-trifluoropropan-2-yl]acetamide |
| 789 | 2-{(3′R,4S)-5′-[1-(2-amino-2-oxoethyl)-1H-pyrazol-4-yl]-3′-fluoro-2,5-dioxo- |
| | 2′,3′-dihydro-1H-spiro[imidazolidine-4,1′-inden]-1-yl}-N-(4-fluorobenzyl)-N- |
| | [(2S)-1,1,1-trifluoropropan-2-yl]acetamide |
| 790 | 2-(5′-bromo-6′-fluoro-3′-hydroxy-2,5-dioxo-2′,3′-dihydro-1H- |
| | spiro[imidazolidine-4,1′-inden]-1-yl)-N-(4-fluorobenzyl)-N-[(2S)-1,1,1- |
| | trifluoropropan-2-yl]acetamide |
| 791 | N-(4-fluorobenzyl)-2-(6′-fluoro-5′-{1-[2-(methylamino)-2-oxoethyl]-1H- |
| | pyrazol-4-yl}-2,5-dioxo-2′,3′-dihydro-1H-spiro[imidazolidine-4,1′-inden]-1- |
| | yl)-N-[(2S)-1,1,1-trifluoropropan-2-yl]acetamide |
| 792 | 2-{5′-[1-(2-amino-2-oxoethyl)-1H-pyrazol-4-yl]-6′-fluoro-2,5-dioxo-2′,3′- |
| | dihydro-1H-spiro[imidazolidine-4,1′-inden]-1-yl}-N-(4-fluorobenzyl)-N-[(2S)- |
| | 1,1,1-trifluoropropan-2-yl]acetamide |
| 793 | N-(4-fluorobenzyl)-2-{6′-fluoro-5′-[1-(2-hydroxyethyl)-1H-pyrazol-4-yl]-2,5- |
| | dioxo-2′,3′-dihydro-1H-spiro[imidazolidine-4,1′-inden]-1-yl}-N-[(2S)-1,1,1- |
| | trifluoropropan-2-yl]acetamide |
| 794 | N-(4-fluorobenzyl)-2-[6′-fluoro-5′-(1-methyl-1H-pyrazol-4-yl)-2,5-dioxo-2′,3′- |
| | dihydro-1H-spiro[imidazolidine-4,1′-inden]-1-yl]-N-[(2S)-1,1,1- |
| | trifluoropropan-2-yl]acetamide |
| 795 | 2-(5′-bromo-3′,6′-difluoro-2,5-dioxo-2′,3′-dihydro-1H-spiro[imidazolidine-4,1′- |
| | inden]-1-yl)-N-(4-fluorobenzyl)-N-[(2S)-1,1,1-trifluoropropan-2-yl]acetamide |
| 796 | tert-butyl 4-[1-(2-{(4-fluorobenzyl)[(2S)-1,1,1-trifluoropropan-2-yl]amino}-2- |
| | oxoethyl)-2,5-dioxo-2′,3′-dihydrospiro[imidazolidine-4,1′-inden]-4′-yl]-3,6- |
| | dihydropyridine-1(2H)-carboxylate |
| 797 | N-(4-fluorobenzyl)-2-{(4S)-7-[(methylcarbamoyl)amino]-2′,4′-dioxo-1H,3′H- |
| | spiro[isochromene-4,5′-[1,3]oxazolidin]-3′-yl}-N-[(2S)-1,1,1-trifluoropropan- |
| | 2-yl]acetamide |
| 798 | 2-[2,5-dioxo-4′-(1,2,3,6-tetrahydropyridin-4-yl)-2′,3′-dihydro-1H- |
| | spiro[imidazolidine-4,1′-inden]-1-yl]-N-(4-fluorobenzyl)-N-[(2S)-1,1,1- |
| | trifluoropropan-2-yl]acetamide |
| 799 | N-(4-fluorobenzyl)-2-{(4R)-7-[(methylcarbamoyl)amino]-2′,4′-dioxo-1H,3′H- |
| | spiro[isochromene-4,5′-[1,3]oxazolidin]-3′-yl}-N-[(2S)-1,1,1-trifluoropropan- |
| | 2-yl]acetamide |
| 800 | 2-{5′-[1-(2-amino-2-oxoethyl)-1H-pyrazol-4-yl]-3′,6′-difluoro-2,5-dioxo-2′,3′- |
| | dihydro-1H-spiro[imidazolidine-4,1′-inden]-1-yl}-N-(4-fluorobenzyl)-N-[(2S)- |
| | 1,1,1-trifluoropropan-2-yl]acetamide |
| 801 | 2-{4-[3′,6′-difluoro-1-(2-{(4-fluorobenzyl)[(2S)-1,1,1-trifluoropropan-2- |
| | yl]amino}-2-oxoethyl)-2,5-dioxo-2′,3′-dihydro-1H-spiro[imidazolidine-4,1′- |
| | inden]-5′-yl]-1H-pyrazol-1-yl}-N,N-dimethylacetamide |
| 802 | 2-[3′,6′-difluoro-5′-(1-methyl-1H-pyrazol-4-yl)-2,5-dioxo-2′,3′-dihydro-1H- |
| | spiro[imidazolidine-4,1′-inden]-1-yl]-N-(4-fluorobenzyl)-N-[(2S)-1,1,1- |
| | trifluoropropan-2-yl]acetamide |
| 803 | 2-{3′,6′-difluoro-5′-[1-(2-hydroxyethyl)-1H-pyrazol-4-yl]-2,5-dioxo-2′,3′- |
| | dihydro-1H-spiro[imidazolidine-4,1′-inden]-1-yl}-N-(4-fluorobenzyl)-N-[(2S)- |
| | 1,1,1-trifluoropropan-2-yl]acetamide |
| 804 | N-(4-fluorobenzyl)-2-[(4R)-5′-(1-methyl-1H-pyrazol-4-yl)-2,5-dioxo-2′,3′- |
| | dihydro-1H-spiro[imidazolidine-4,1′-inden]-1-yl]-N-[(2S)-1,1,1- |
| | trifluoropropan-2-yl]acetamide |
| 805 | tert-butyl (3R)-3-[(4-fluorobenzyl)({(1R)-5-[(methylcarbamoyl)amino]-2′,4′- |
| | dioxo-2,3-dihydro-3′H-spiro[indene-1,5′-[1,3]oxazolidin]-3′- |
| | yl}acetyl)amino]piperidine-1-carboxylate |
| 806 | tert-butyl (3S)-3-[(4-fluorobenzyl)({(1R)-5-[(methylcarbamoyl)amino]-2′,4′- |
| | dioxo-2,3-dihydro-3′H-spiro[indene-1,5′-[1,3]oxazolidin]-3′- |
| | yl}acetyl)amino]piperidine-1-carboxylate |
| 807 | N-(4-fluorobenzyl)-2-{(1R)-5-[(methylcarbamoyl)amino]-2′,4′-dioxo-2,3- |
| | dihydro-3′H-spiro[indene-1,5′-[1,3]oxazolidin]-3′-yl}-N-[(3R)-piperidin-3- |
| | yl]acetamide |
| 808 | N-(4-fluorobenzyl)-2-{(1R)-5-[(methylcarbamoyl)amino]-2′,4′-dioxo-2,3- |
| | dihydro-3′H-spiro[indene-1,5′-[1,3]oxazolidin]-3′-yl}-N-[(3S)-piperidin-3- |
| | yl]acetamide |
| 809 | N-[4-amino-3-(hydroxymethyl)butan-2-yl]-N-(4-fluorobenzyl)-2-{(1R)-5- |
| | [(methylcarbamoyl)amino]-2′,4′-dioxo-2,3-dihydro-3′H-spiro[indene-1,5′- |
| | [1,3]oxazolidin]-3′-yl}acetamide |
| 810 | ethyl ({(3S)-3-[(4-fluorobenzyl)({(1R)-5-[(methylcarbamoyl)amino]-2′,4′- |
| | dioxo-2,3-dihydro-3′H-spiro[indene-1,5′-[1,3]oxazolidin]-3′- |
| | yl}acetyl)amino]piperidin-1-yl}sulfonyl)carbamate |
| 811 | 4-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′-yl}acetyl)amino]benzamide |
| 812 | tert-butyl 2-{[(4-fluorobenzyl)({(1R)-5-[(methylcarbamoyl)amino]-2′,4′- |
| | dioxo-2,3-dihydro-3′H-spiro[indene-1,5′-[1,3]oxazolidin]-3′- |
| | yl}acetyl)amino]methyl}pyrrolidine-1-carboxylate |
| 813 | N-(4-fluorobenzyl)-2-{(1R)-5-[(methylcarbamoyl)amino]-2′,4′-dioxo-2,3- |
| | dihydro-3′H-spiro[indene-1,5′-[1,3]oxazolidin]-3′-yl}-N-(pyrrolidin-2- |
| | ylmethyl)acetamide |
| 814 | tert-butyl {4-[(4-fluorobenzyl)({(1R)-5-[(methylcarbamoyl)amino]-2′,4′- |
| | dioxo-2,3-dihydro-3′H-spiro[indene-1,5′-[1,3]oxazolidin]-3′- |
| | yl}acetyl)amino]cyclohexylidene}acetate |
| 815 | N-ethyl-4-[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′- |
| | yl}acetyl)amino]cyclohexanecarboxamide |
| 816 | {4-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′- |
| | yl}acetyl)amino]cyclohexylidene}acetic acid |
| 817 | 2-[(4S)-2,5-dioxo-5′-(1H-pyrazol-4-yl)-2′,3′-dihydro-1H-spiro[imidazolidine- |
| | 4,1′-inden]-1-yl]-N-(4-fluorobenzyl)-N-[(2S)-1,1,1-trifluoropropan-2- |
| | yl]acetamide |
| 818 | N-(4-fluorobenzyl)-2-{5′-[(1-methyl-1H-pyrazol-3-yl)amino]-2,5-dioxo-2′,3′- |
| | dihydro-1H-spiro[imidazolidine-4,1′-inden]-1-yl}-N-[(2S)-1,1,1- |
| | trifluoropropan-2-yl]acetamide |
| 819 | 2-[(4S)-5′-amino-2,5-dioxo-2′,3′-dihydro-1H-spiro[imidazolidine-4,1′-inden]- |
| | 1-yl]-N-(4-fluorobenzyl)-N-[(2S)-1,1,1-trifluoropropan-2-yl]acetamide |
| 820 | 2-{4′-[(dimethylamino)methyl]-2,5-dioxo-2′,3′-dihydro-1H- |
| | spiro[imidazolidine-4,1′-inden]-1-yl}-N-(4-fluorobenzyl)-N-[(2S)-1,1,1- |
| | trifluoropropan-2-yl]acetamide |
| 821 | N-(4-fluorobenzyl)-2-[4′-(morpholin-4-ylmethyl)-2,5-dioxo-2′,3′-dihydro-1H- |
| | spiro[imidazolidine-4,1′-inden]-1-yl]-N-[(2S)-1,1,1-trifluoropropan-2- |
| | yl]acetamide |
| 822 | 2-[2,5-dioxo-4′-(pyrrolidin-1-ylmethyl)-2′,3′-dihydro-1H-spiro[imidazolidine- |
| | 4,1′-inden]-1-yl]-N-(4-fluorobenzyl)-N-[(2S)-1,1,1-trifluoropropan-2- |
| | yl]acetamide |
| 823 | 2-(4′-amino-2,5-dioxo-2′,3′-dihydro-1H-spiro[imidazolidine-4,1′-inden]-1-yl)- |
| | N-(4-fluorobenzyl)-N-[(2S)-1,1,1-trifluoropropan-2-yl]acetamide |
| 824 | N-(4-fluorobenzyl)-2-[(1R)-5-(oxetan-3-ylamino)-2′,4′-dioxo-2,3-dihydro-3′H- |
| | spiro[indene-1,5′-[1,3]oxazolidin]-3′-yl]-N-[(2S)-1,1,1-trifluoropropan-2- |
| | yl]acetamide |
| 825 | 3-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′-yl}acetyl)amino]benzamide |
| 826 | N-[(1-acetylpyrrolidin-2-yl)methyl]-N-(4-fluorobenzyl)-2-{(1R)-5- |
| | [(methylcarbamoyl)amino]-2′,4′-dioxo-2,3-dihydro-3′H-spiro[indene-1,5′- |
| | [1,3]oxazolidin]-3′-yl}acetamide |
| 827 | N-(4-fluorobenzyl)-2-{(1R)-5-[(methylcarbamoyl)amino]-2′,4′-dioxo-2,3- |
| | dihydro-3′H-spiro[indene-1,5′-[1,3]oxazolidin]-3′-yl}-N-{[1- |
| | (methylsulfonyl)pyrrolidin-2-yl]methyl}acetamide |
| 828 | 2-{[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′-yl}acetyl)amino]methyl}-N- |
| | methylpyrrolidine-1-carboxamide |
| 829 | N-(3,4-difluorobenzyl)-2-[(1R)-5-{1-[2-(methylamino)-2-oxoethyl]-1H- |
| | pyrazol-4-yl}-2′,4′-dioxo-2,3-dihydro-3′H-spiro[indene-1,5′-[1,3]oxazolidin]- |
| | 3′-yl]-N-[(2S)-1,1,1-trifluoropropan-2-yl]acetamide |
| 830 | N-(4-fluorobenzyl)-2-{(1R)-5-[(methylcarbamoyl)amino]-2′,4′-dioxo-2,3- |
| | dihydro-3′H-spiro[indene-1,5′-[1,3]oxazolidin]-3′-yl}-N-{[1- |
| | (methylsulfamoyl)pyrrolidin-2-yl]methyl}acetamide |
| 831 | N-(cyclopropylmethyl)-N-(2,3-dihydro-1H-inden-1-yl)-2-{(1R)-5- |
| | [(methylcarbamoyl)amino]-2′,4′-dioxo-2,3-dihydro-3′H-spiro[indene-1,5′- |
| | [1,3]oxazolidin]-3′-yl}acetamide |
| 832 | methyl trans-4-{[(5′-bromo-2,5-dioxo-2′,3′-dihydro-1H-spiro[imidazolidine- |
| | 4,1′-inden]-1-yl)acetyl](4-fluorobenzyl)amino}cyclohexanecarboxylate |
| 833 | trans-4-{[(5′-bromo-2,5-dioxo-2′,3′-dihydro-1H-spiro[imidazolidine-4,1′- |
| | inden]-1-yl)acetyl](4-fluorobenzyl)amino}cyclohexanecarboxylic acid |
| 834 | trans-4-[(4-fluorobenzyl){[5′-(1-methyl-1H-pyrazol-4-yl)-2,5-dioxo-2′,3′- |
| | dihydro-1H-spiro[imidazolidine-4,1′-inden]-1- |
| | yl]acetyl}amino]cyclohexanecarboxylic acid |
| 835 | 2-[(4R)-7′-bromo-2,5-dioxo-2′,3′-dihydro-1H-spiro[imidazolidine-4,1′-inden]- |
| | 1-yl]-N-(4-fluorobenzyl)-N-[(2S)-1,1,1-trifluoropropan-2-yl]acetamide |
| 836 | trans-4-{(4-fluorobenzyl)[(5′-{1-[2-(methylamino)-2-oxoethyl]-1H-pyrazol-4- |
| | yl}-2,5-dioxo-2′,3′-dihydro-1H-spiro[imidazolidine-4,1′-inden]-1- |
| | yl)acetyl]amino}cyclohexanecarboxylic acid |
| 837 | 2-[(4S)-7′-bromo-2,5-dioxo-2′,3′-dihydro-1H-spiro[imidazolidine-4,1′-inden]- |
| | 1-yl]-N-(4-fluorobenzyl)-N-[(2S)-1,1,1-trifluoropropan-2-yl]acetamide |
| 838 | ethyl ({4-[(4-fluorobenzyl){[5′-(1-methyl-1H-pyrazol-4-yl)-2,5-dioxo-2′,3′- |
| | dihydro-1H-spiro[imidazolidine-4,1′-inden]-1-yl]acetyl}amino]piperidin-1- |
| | yl}sulfonyl)carbamate |
| 839 | ethyl ({(3R)-3-[(4-fluorobenzyl)({(1R)-5-[(methylcarbamoyl)amino]-2′,4′- |
| | dioxo-2,3-dihydro-3′H-spiro[indene-1,5′-[1,3]oxazolidin]-3′- |
| | yl}acetyl)amino]piperidin-1-yl}sulfonyl)carbamate |
| 840 | {4-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′- |
| | yl}acetyl)amino]cyclohexyl}acetic acid |
| 841 | 2-[(4S)-5′-{[(2-cyanoethyl)carbamoyl]amino}-2,5-dioxo-2′,3′-dihydro-1H- |
| | spiro[imidazolidine-4,1′-inden]-1-yl]-N-(4-fluorobenzyl)-N-[(2S)-1,1,1- |
| | trifluoropropan-2-yl]acetamide |
| 842 | N-(4-fluorobenzyl)-2-{(4S)-5′-[(methylcarbamoyl)amino]-2,5-dioxo-2′,3′- |
| | dihydro-1H-spiro[imidazolidine-4,1′-inden]-1-yl}-N-[(2S)-1,1,1- |
| | trifluoropropan-2-yl]acetamide |
| 843 | N-(4-fluorobenzyl)-2-{(4S)-5′-[(methylcarbamoyl)amino]-2,3′,5-trioxo-2′,3′- |
| | dihydro-1H-spiro[imidazolidine-4,1′-inden]-1-yl}-N-[(2S)-1,1,1- |
| | trifluoropropan-2-yl]acetamide |
| 844 | N-(4-fluorobenzyl)-2-{(3′S,4S)-3′-hydroxy-5′-[(methylcarbamoyl)amino]-2,5- |
| | dioxo-2′,3′-dihydro-1H-spiro[imidazolidine-4,1′-inden]-1-yl}-N-[(2S)-1,1,1- |
| | trifluoropropan-2-yl]acetamide |
| 845 | N-(4-fluorobenzyl)-2-{(3′R,4S)-3′-fluoro-5′-[1-(2-hydroxyethyl)-1H-pyrazol- |
| | 4-yl]-2,5-dioxo-2′,3′-dihydro-1H-spiro[imidazolidine-4,1′-inden]-1-yl}-N- |
| | [(2S)-1,1,1-trifluoropropan-2-yl]acetamide |
| 846 | ethyl ({3-[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′-yl}acetyl)amino]-3- |
| | methylazetidin-1-yl}sulfonyl)carbamate |
| 847 | 2-[4′-(acetylamino)-2,5-dioxo-2′,3′-dihydro-1H-spiro[imidazolidine-4,1′- |
| | inden]-1-yl]-N-(4-fluorobenzyl)-N-[(2S)-1,1,1-trifluoropropan-2-yl]acetamide |
| 848 | 2-[(4R)-5′-bromo-6′-fluoro-2,3′,5-trioxo-2′,3′-dihydro-1H-spiro[imidazolidine- |
| | 4,1′-inden]-1-yl]-N-(4-fluorobenzyl)-N-[(2S)-1,1,1-trifluoropropan-2- |
| | yl]acetamide |
| 849 | N-(3-cyanophenyl)-N-(4-fluorobenzyl)-2-{(1R)-5-[(methylcarbamoyl)amino]- |
| | 2′,4′-dioxo-2,3-dihydro-3′H-spiro[indene-1,5′-[1,3]oxazolidin]-3′-yl}acetamide |
| 850 | N-(4-fluorobenzyl)-2-{(3′R,4S)-3′-fluoro-5′-[(methylcarbamoyl)amino]-2,5- |
| | dioxo-2′,3′-dihydro-1H-spiro[imidazolidine-4,1′-inden]-1-yl}-N-[(2S)-1,1,1- |
| | trifluoropropan-2-yl]acetamide |
| 851 | N-(3,4-difluorobenzyl)-2-{(1R)-5-[(methylcarbamoyl)amino]-2′,4′-dioxo-2,3- |
| | dihydro-3′H-spiro[indene-1,5′-[1,3]oxazolidin]-3′-yl}-N-[(2S)-1,1,1- |
| | trifluoropropan-2-yl]acetamide |
| 852 | N-(4-fluorobenzyl)-2-{7′-[(methylcarbamoyl)amino]-2,2′,5-trioxo-2′,3′- |
| | dihydro-1H,1′H-spiro[imidazolidine-4,4′-quinolin]-1-yl}-N-[(2S)-1,1,1- |
| | trifluoropropan-2-yl]acetamide |
| 853 | tert-butyl (4-{[(5′-bromo-2,5-dioxo-2′,3′-dihydro-1H-spiro[imidazolidine-4,1′- |
| | inden]-1-yl)acetyl](4-fluorobenzyl)amino}cyclohexylidene)acetate |
| 854 | (4-{[(5′-bromo-2,5-dioxo-2′,3′-dihydro-1H-spiro[imidazolidine-4,1′-inden]-1- |
| | yl)acetyl](4-fluorobenzyl)amino}cyclohexylidene)acetic acid |
| 855 | {4-[(4-fluorobenzyl){[5′-(1-methyl-1H-pyrazol-4-yl)-2,5-dioxo-2′,3′-dihydro- |
| | 1H-spiro[imidazolidine-4,1′-inden]-1-yl]acetyl}amino]cyclohexylidene}acetic |
| | acid |
| 856 | (4-{(4-fluorobenzyl)[(5′-{1-[2-(methylamino)-2-oxoethyl]-1H-pyrazol-4-yl}- |
| | 2,5-dioxo-2′,3′-dihydro-1H-spiro[imidazolidine-4,1′-inden]-1- |
| | yl)acetyl]amino}cyclohexylidene)acetic acid |
| 857 | tert-butyl {3-[(4-fluorobenzyl)({(1R)-5-[(methylcarbamoyl)amino]-2′,4′- |
| | dioxo-2,3-dihydro-3′H-spiro[indene-1,5′-[1,3]oxazolidin]-3′- |
| | yl}acetyl)amino]cyclobutyl}carbamate |
| 858 | N-(3-aminocyclobutyl)-N-(4-fluorobenzyl)-2-{(1R)-5- |
| | [(methylcarbamoyl)amino]-2′,4′-dioxo-2,3-dihydro-3′H-spiro[indene-1,5′- |
| | [1,3]oxazolidin]-3′-yl}acetamide |
| 859 | N-[3-(acetylamino)cyclobutyl]-N-(4-fluorobenzyl)-2-{(1R)-5- |
| | [(methylcarbamoyl)amino]-2′,4′-dioxo-2,3-dihydro-3′H-spiro[indene-1,5′- |
| | [1,3]oxazolidin]-3′-yl}acetamide |
| 860 | N-(4-fluorobenzyl)-2-{(1R)-5-[(methylcarbamoyl)amino]-2′,4′-dioxo-2,3- |
| | dihydro-3′H-spiro[indene-1,5′-[1,3]oxazolidin]-3′-yl}-N-{3- |
| | [(methylsulfonyl)amino]cyclobutyl}acetamide |
| 861 | N-(4-fluorobenzyl)-2-[(3′R,4S)-3′-hydroxy-5′-(1-methyl-1H-pyrazol-4-yl)-2,5- |
| | dioxo-2′,3′-dihydro-1H-spiro[imidazolidine-4,1′-inden]-1-yl]-N-[(2S)-1,1,1- |
| | trifluoropropan-2-yl]acetamide |
| 862 | 2-[(4R)-5′-(acetylamino)-2,5-dioxo-2′,3′-dihydro-1H-spiro[imidazolidine-4,1′- |
| | inden]-1-yl]-N-(4-fluorobenzyl)-N-[(2S)-1,1,1-trifluoropropan-2-yl]acetamide |
| 863 | N-(4-fluorobenzyl)-2-[(3′E,4S)-3′-(hydroxyimino)-5′-(1-methyl-1H-pyrazol-4- |
| | yl)-2,5-dioxo-2′,3′-dihydro-1H-spiro[imidazolidine-4,1′-inden]-1-yl]-N-[(2S)- |
| | 1,1,1-trifluoropropan-2-yl]acetamide |
| 864 | 2,2′-({3-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′- |
| | yl}acetyl)amino]cyclobutyl}imino)diacetic acid |
| 865 | N-{3-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′- |
| | yl}acetyl)amino]cyclobutyl}glycine |
| 866 | tert-butyl 3-[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′- |
| | yl}acetyl)amino]cyclobutanecarboxylate |
| 867 | methyl {3-[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′- |
| | yl}acetyl)amino]cyclobutyl}carbamate |
| 868 | ethyl ({3-[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′- |
| | yl}acetyl)amino]cyclobutyl}sulfamoyl)carbamate |
| 869 | 3-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′- |
| | yl}acetyl)amino]cyclobutanecarboxylic acid |
| 870 | 2-[(3′R,4S)-3′,6′-difluoro-5′-{1-[2-(methylamino)-2-oxoethyl]-1H-pyrazol-4- |
| | yl}-2,5-dioxo-2′,3′-dihydro-1H-spiro[imidazolidine-4,1′-inden]-1-yl]-N-(4- |
| | fluorobenzyl)-N-[(2S)-1,1,1-trifluoropropan-2-yl]acetamide |
| 871 | 2-{4-[(3′R,4S)-3′,6′-difluoro-1-(2-{(4-fluorobenzyl)[(2S)-1,1,1- |
| | trifluoropropan-2-yl]amino}-2-oxoethyl)-2,5-dioxo-2′,3′-dihydro-1H- |
| | spiro[imidazolidine-4,1′-inden]-5′-yl]-1H-pyrazol-1-yl}-N,N- |
| | dimethylacetamide |
| 872 | 2-[(3′S,4R)-3′,6′-difluoro-5′-{1-[2-(methylamino)-2-oxoethyl]-1H-pyrazol-4- |
| | yl}-2,5-dioxo-2′,3′-dihydro-1H-spiro[imidazolidine-4,1′-inden]-1-yl]-N-(4- |
| | fluorobenzyl)-N-[(2S)-1,1,1-trifluoropropan-2-yl]acetamide |
| 873 | 2-{4-[(3′S,4R)-3′,6′-difluoro-1-(2-{(4-fluorobenzyl)[(2S)-1,1,1- |
| | trifluoropropan-2-yl]amino}-2-oxoethyl)-2,5-dioxo-2′,3′-dihydro-1H- |
| | spiro[imidazolidine-4,1′-inden]-5′-yl]-1H-pyrazol-1-yl}-N,N- |
| | dimethylacetamide |
| 874 | ethyl [(4-{(4-fluorobenzyl)[(5′-{1-[2-(methylamino)-2-oxoethyl]-1H-pyrazol- |
| | 4-yl}-2,5-dioxo-2′,3′-dihydro-1H-spiro[imidazolidine-4,1′-inden]-1- |
| | yl)acetyl]amino}piperidin-1-yl)sulfonyl]carbamate |
| 875 | ethyl ({4-[(4-fluorobenzyl){[(1R)-5-(1-methyl-1H-pyrazol-4-yl)-2′,4′-dioxo- |
| | 2,3-dihydro-3′H-spiro[indene-1,5′-[1,3]oxazolidin]-3′- |
| | yl]acetyl}amino]piperidin-1-yl}sulfonyl)carbamate |
| 876 | 2-[(1S,3S)-5-bromo-3-hydroxy-2′,4′-dioxo-2,3-dihydro-3′H-spiro[indene-1,5′- |
| | [1,3]oxazolidin]-3′-yl]-N-(4-fluorobenzyl)-N-[(2S)-1,1,1-trifluoropropan-2- |
| | yl]acetamide |
| 877 | 2-(5-chloro-4-cyano-2′,4′-dioxo-2,3-dihydro-3′H-spiro[indene-1,5′- |
| | [1,3]oxazolidin]-3′-yl)-N-(4-fluorobenzyl)-N-[(2S)-1,1,1-trifluoropropan-2- |
| | yl]acetamide |
| 878 | benzyl 3-{[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′- |
| | yl}acetyl)amino]methyl}azetidine-1-carboxylate |
| 879 | tert-butyl 4-[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′- |
| | yl}acetyl)amino]benzoate |
| 880 | tert-butyl 4-[{[(4S)-5′-bromo-2,5-dioxo-2′,3′-dihydro-1H-spiro[imidazolidine- |
| | 4,1′-inden]-1-yl]acetyl}(4-fluorobenzyl)amino]piperidine-1-carboxylate |
| 881 | N-(4-fluorobenzyl)-2-[(1S,3S)-3-hydroxy-5-{1-[2-(methylamino)-2-oxoethyl]- |
| | 1H-pyrazol-4-yl}-2′,4′-dioxo-2,3-dihydro-3′H-spiro[indene-1,5′- |
| | [1,3]oxazolidin]-3′-yl]-N-[(2S)-1,1,1-trifluoropropan-2-yl]acetamide |
| 882 | ethyl ({4-[{[(4S)-5′-amino-2,5-dioxo-2′,3′-dihydro-1H-spiro[imidazolidine- |
| | 4,1′-inden]-1-yl]acetyl}(4-fluorobenzyl)amino]piperidin-1- |
| | yl}sulfonyl)carbamate |
| 883 | ethyl ({3,3-difluoro-4-[(4-fluorobenzyl){[(4S)-5′-(1-methyl-1H-pyrazol-4-yl)- |
| | 2,5-dioxo-2′,3′-dihydro-1H-spiro[imidazolidine-4,1′-inden]-1- |
| | yl]acetyl}amino]pyrrolidin-1-yl}sulfonyl)carbamate |
| 884 | ethyl ({3,3-difluoro-4-[(4-fluorobenzyl){[(4S)-5′-{1-[2-(methylamino)-2- |
| | oxoethyl]-1H-pyrazol-4-yl}-2,5-dioxo-2′,3′-dihydro-1H-spiro[imidazolidine- |
| | 4,1′-inden]-1-yl]acetyl}amino]pyrrolidin-1-yl}sulfonyl)carbamate |
| 885 | ethyl ({3,3-difluoro-4-[(4-fluorobenzyl){[(1R)-5-(1-methyl-1H-pyrazol-4-yl)- |
| | 2′,4′-dioxo-2,3-dihydro-3′H-spiro[indene-1,5′-[1,3]oxazolidin]-3′- |
| | yl]acetyl}amino]pyrrolidin-1-yl}sulfonyl)carbamate |
| 886 | ethyl ({3,3-difluoro-4-[(4-fluorobenzyl){[(1R)-5-{1-[2-(methylamino)-2- |
| | oxoethyl]-1H-pyrazol-4-yl}-2′,4′-dioxo-2,3-dihydro-3′H-spiro[indene-1,5′- |
| | [1,3]oxazolidin]-3′-yl]acetyl}amino]pyrrolidin-1-yl}sulfonyl)carbamate |
| 887 | N-(4-fluorobenzyl)-2-[(1S)-3-fluoro-5-{1-[2-(methylamino)-2-oxoethyl]-1H- |
| | pyrazol-4-yl}-2′,4′-dioxo-2,3-dihydro-3′H-spiro[indene-1,5′-[1,3]oxazolidin]- |
| | 3′-yl]-N-[(2S)-1,1,1-trifluoropropan-2-yl]acetamide |
| 888 | 2-[(1S)-5-bromo-3-fluoro-2′,4′-dioxo-2,3-dihydro-3′H-spiro[indene-1,5′- |
| | [1,3]oxazolidin]-3′-yl]-N-(4-fluorobenzyl)-N-[(2S)-1,1,1-trifluoropropan-2- |
| | yl]acetamide |
| 889 | N-(4-fluorobenzyl)-N-(3-methoxycyclobutyl)-2-{(1R)-5- |
| | [(methylcarbamoyl)amino]-2′,4′-dioxo-2,3-dihydro-3′H-spiro[indene-1,5′- |
| | [1,3]oxazolidin]-3′-yl}acetamide |
| 890 | 3-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′-yl}acetyl)amino]-N- |
| | methylcyclobutanecarboxamide |
| 891 | 3-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′- |
| | yl}acetyl)amino]cyclobutanecarboxamide |
| 892 | 2-{(3′R,4S)-5′-[1-(difluoromethyl)-1H-pyrazol-4-yl]-3′-fluoro-2,5-dioxo-2′,3′- |
| | dihydro-1H-spiro[imidazolidine-4,1′-inden]-1-yl}-N-(4-fluorobenzyl)-N-[(2S)- |
| | 1,1,1-trifluoropropan-2-yl]acetamide |
| 893 | 2-(5-amino-4-cyano-2′,4′-dioxo-2,3-dihydro-3′H-spiro[indene-1,5′- |
| | [1,3]oxazolidin]-3′-yl)-N-(4-fluorobenzyl)-N-[(2S)-1,1,1-trifluoropropan-2- |
| | yl]acetamide |
| 894 | 2-[2,5-dioxo-4′-(piperidin-4-yl)-2′,3′-dihydro-1H-spiro[imidazolidine-4,1′- |
| | inden]-1-yl]-N-(4-fluorobenzyl)-N-[(2S)-1,1,1-trifluoropropan-2-yl]acetamide |
| 895 | tert-butyl 3-[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′- |
| | yl}acetyl)amino]benzoate |
| 896 | 4-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′-yl}acetyl)amino]benzoic |
| | acid |
| 897 | tert-butyl {4-[(4-fluorobenzyl)({(1R)-5-[(methylcarbamoyl)amino]-2′,4′- |
| | dioxo-2,3-dihydro-3′H-spiro[indene-1,5′-[1,3]oxazolidin]-3′- |
| | yl}acetyl)amino]phenyl}acetate |
| 898 | 3-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′-yl}acetyl)amino]benzoic |
| | acid |
| 899 | {4-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′- |
| | yl}acetyl)amino]phenyl}acetic acid |
| 900 | (2-{[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′- |
| | yl}acetyl)amino]methyl}pyrrolidin-1-yl)acetic acid |
| 901 | methyl {3-[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′- |
| | yl}acetyl)amino]phenyl}acetate |
| 902 | N-(4-fluorobenzyl)-2-[(4S)-7′-hydroxy-2,5-dioxo-2′,3′-dihydro-1H- |
| | spiro[imidazolidine-4,1′-inden]-1-yl]-N-[(2S)-1,1,1-trifluoropropan-2- |
| | yl]acetamide |
| 903 | {3-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′- |
| | yl}acetyl)amino]phenyl}acetic acid |
| 904 | tert-butyl {4-[{[(1R)-5-bromo-2′,4′-dioxo-2,3-dihydro-3′H-spiro[indene-1,5′- |
| | [1,3]oxazolidin]-3′-yl]acetyl}(4-fluorobenzyl)amino]cyclohexylidene}acetate |
| 905 | tert-butyl {4-[{[(4S)-5′-bromo-2,5-dioxo-2′,3′-dihydro-1H-spiro[imidazolidine- |
| | 4,1′-inden]-1-yl]acetyl}(4-fluorobenzyl)amino]cyclohexylidene}acetate |
| 906 | tert-butyl {4-[(4-fluorobenzyl){[(1S)-5-(1-methyl-1H-pyrazol-4-yl)-2′,4′- |
| | dioxo-2,3-dihydro-3′H-spiro[indene-1,5′-[1,3]oxazolidin]-3′- |
| | yl]acetyl}amino]cyclohexylidene}acetate |
| 907 | tert-butyl {4-[(4-fluorobenzyl){[(4S)-5′-(1-methyl-1H-pyrazol-4-yl)-2,5-dioxo- |
| | 2′,3′-dihydro-1H-spiro[imidazolidine-4,1′-inden]-1- |
| | yl]acetyl}amino]cyclohexylidene}acetate |
| 908 | {4-[(4-fluorobenzyl){[(1R)-5-(1-methyl-1H-pyrazol-4-yl)-2′,4′-dioxo-2,3- |
| | dihydro-3′H-spiro[indene-1,5′-[1,3]oxazolidin]-3′- |
| | yl]acetyl}amino]cyclohexylidene}acetic acid |
| 909 | {4-[{[(4S)-5′-amino-2,5-dioxo-2′,3′-dihydro-1H-spiro[imidazolidine-4,1′- |
| | inden]-1-yl]acetyl}(4-fluorobenzyl)amino]cyclohexylidene}acetic acid |
| 910 | N-(4-fluorobenzyl)-2-[(1S,3R)-3-fluoro-5-(1-methyl-1H-pyrazol-4-yl)-2′,4′- |
| | dioxo-2,3-dihydro-3′H-spiro[indene-1,5′-[1,3]oxazolidin]-3′-yl]-N-[(2S)-1,1,1- |
| | trifluoropropan-2-yl]acetamide |
| 911 | 2-[(4S)-5′-bromo-6′-fluoro-2,3′,5-trioxo-2′,3′-dihydro-1H-spiro[imidazolidine- |
| | 4,1′-inden]-1-yl]-N-(4-fluorobenzyl)-N-[(2S)-1,1,1-trifluoropropan-2- |
| | yl]acetamide |
| 912 | 2-[(3′R,4R)-5′-bromo-6′-fluoro-3′-hydroxy-2,5-dioxo-2′,3′-dihydro-1H- |
| | spiro[imidazolidine-4,1′-inden]-1-yl]-N-(4-fluorobenzyl)-N-[(2S)-1,1,1- |
| | trifluoropropan-2-yl]acetamide |
| 913 | 2-[(3′S,4S)-5′-bromo-6′-fluoro-3′-hydroxy-2,5-dioxo-2′,3′-dihydro-1H- |
| | spiro[imidazolidine-4,1′-inden]-1-yl]-N-(4-fluorobenzyl)-N-[(2S)-1,1,1- |
| | trifluoropropan-2-yl]acetamide |
| 914 | N-(4-fluorobenzyl)-2-[(3′R,4S)-3′-fluoro-5′-(1-methyl-1H-pyrazol-4-yl)-2,5- |
| | dioxo-2′,3′-dihydro-1H-spiro[imidazolidine-4,1′-inden]-1-yl]-N-(tetrahydro- |
| | 2H-pyran-4-yl)acetamide |
| 915 | 2-(2,5-dioxo-2′,3′-dihydro-1H-spiro[imidazolidine-4,1′-inden]-1-yl)-N-(4- |
| | fluorobenzyl)-N-[(2S)-1,1,1-trifluoropropan-2-yl]acetamide |
| 916 | N-(4-fluorobenzyl)-2-{6′-fluoro-5′-[(methylcarbamoyl)amino]-2,5-dioxo-2′,3′- |
| | dihydro-1H-spiro[imidazolidine-4,1′-inden]-1-yl}-N-[(2S)-1,1,1- |
| | trifluoropropan-2-yl]acetamide |
| 917 | 2-{(3′R,4S)-3′,6′-difluoro-5′-[(methylcarbamoyl)amino]-2,5-dioxo-2′,3′- |
| | dihydro-1H-spiro[imidazolidine-4,1′-inden]-1-yl}-N-(4-fluorobenzyl)-N-[(2S)- |
| | 1,1,1-trifluoropropan-2-yl]acetamide |
| 918 | N-(azetidin-3-ylmethyl)-N-(4-fluorobenzyl)-2-{(1R)-5- |
| | [(methylcarbamoyl)amino]-2′,4′-dioxo-2,3-dihydro-3′H-spiro[indene-1,5′- |
| | [1,3]oxazolidin]-3′-yl}acetamide |
| 919 | {4-[{[(1R)-2′,4′-dioxo-2,3-dihydro-3′H-spiro[indene-1,5′-[1,3]oxazolidin]-3′- |
| | yl]acetyl}(4-fluorobenzyl)amino]cyclohexylidene}acetic acid |
| 920 | methyl {4-[(4-fluorobenzyl){[(4S)-5′-(1-methyl-1H-pyrazol-4-yl)-2,5-dioxo- |
| | 2′,3′-dihydro-1H-spiro[imidazolidine-4,1′-inden]-1- |
| | yl]acetyl}amino]cyclohexylidene}acetate |
| 921 | {4-[(4-fluorobenzyl){[(3′R,4S)-3′-fluoro-5′-(1-methyl-1H-pyrazol-4-yl)-2,5- |
| | dioxo-2′,3′-dihydro-1H-spiro[imidazolidine-4,1′-inden]-1- |
| | yl]acetyl}amino]cyclohexylidene}acetic acid |
| 922 | tert-butyl {trans-4-[(4-fluorobenzyl)({(1R)-5-[(methylcarbamoyl)amino]-2′,4′- |
| | dioxo-2,3-dihydro-3′H-spiro[indene-1,5′-[1,3]oxazolidin]-3′- |
| | yl}acetyl)amino]cyclohexyl}carbamate |
| 923 | N-(trans-4-aminocyclohexyl)-N-(4-fluorobenzyl)-2-{(1R)-5- |
| | [(methylcarbamoyl)amino]-2′,4′-dioxo-2,3-dihydro-3′H-spiro[indene-1,5′- |
| | [1,3]oxazolidin]-3′-yl}acetamide |
| 924 | N-(4-fluorobenzyl)-2-{(1R)-5-[(methylcarbamoyl)amino]-2′,4′-dioxo-2,3- |
| | dihydro-3′H-spiro[indene-1,5′-[1,3]oxazolidin]-3′-yl}-N-{trans-4- |
| | [(methylsulfonyl)amino]cyclohexyl}acetamide |
| 925 | N-(4-fluorobenzyl)-2-{(1R)-5-[(methylcarbamoyl)amino]-2′,4′-dioxo-2,3- |
| | dihydro-3′H-spiro[indene-1,5′-[1,3]oxazolidin]-3′-yl}-N-(trans-4- |
| | {[(trifluoromethyl)sulfonyl]amino}cyclohexyl)acetamide |
| 926 | N-(4-fluorobenzyl)-2-[(4R)-7′-hydroxy-2,5-dioxo-2′,3′-dihydro-1H- |
| | spiro[imidazolidine-4,1′-inden]-1-yl]-N-[(2S)-1,1,1-trifluoropropan-2- |
| | yl]acetamide |
| 927 | tert-butyl {4-[(4-fluorobenzyl){[(4S)-5′-(1-methyl-1H-pyrazol-4-yl)-2,5-dioxo- |
| | 2′,3′-dihydro-1H-spiro[imidazolidine-4,1′-inden]-1- |
| | yl]acetyl}amino]cyclohexyl}acetate |
| 928 | {4-[(4-fluorobenzyl){[(4S)-5′-(1-methyl-1H-pyrazol-4-yl)-2,5-dioxo-2′,3′- |
| | dihydro-1H-spiro[imidazolidine-4,1′-inden]-1- |
| | yl]acetyl}amino]cyclohexyl}acetic acid |
| 929 | tert-butyl 6-[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′-yl}acetyl)amino]-2- |
| | azaspiro[3.3]heptane-2-carboxylate |
| 930 | N-(2-azaspiro[3.3]hept-6-yl)-N-(4-fluorobenzyl)-2-{(1R)-5- |
| | [(methylcarbamoyl)amino]-2′,4′-dioxo-2,3-dihydro-3′H-spiro[indene-1,5′- |
| | [1,3]oxazolidin]-3′-yl}acetamide |
| 931 | 2-[(1S,3R)-5-bromo-3-hydroxy-2′,4′-dioxo-2,3-dihydro-3′H-spiro[indene-1,5′- |
| | [1,3]oxazolidin]-3′-yl]-N-(4-fluorobenzyl)-N-[(2S)-1,1,1-trifluoropropan-2- |
| | yl]acetamide |
| 932 | N-[(1-acetylazetidin-3-yl)methyl]-N-(4-fluorobenzyl)-2-{(1R)-5- |
| | [(methylcarbamoyl)amino]-2′,4′-dioxo-2,3-dihydro-3′H-spiro[indene-1,5′- |
| | [1,3]oxazolidin]-3′-yl}acetamide |
| 933 | N-(4-fluorobenzyl)-N-[(2S)-1,1,1-trifluoropropan-2-yl]-2-(2′,3,4′-trioxo- |
| | 1,2,3,4,8,9-hexahydro-3′H-spiro[cyclopenta[f]quinazoline-7,5′- |
| | [1,3]oxazolidin]-3′-yl)acetamide |
| 934 | ethyl [(3-{[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′- |
| | yl}acetyl)amino]methyl}azetidin-1-yl)sulfonyl]carbamate |
| 935 | ethyl [(2-{[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′- |
| | yl}acetyl)amino]methyl}pyrrolidin-1-yl)sulfonyl]carbamate |
| 936 | 2-[(4S)-2,5-dioxo-5′-(1H-pyrazol-5-yl)-2′,3′-dihydro-1H-spiro[imidazolidine- |
| | 4,1′-inden]-1-yl]-N-(4-fluorobenzyl)-N-[(2S)-1,1,1-trifluoropropan-2- |
| | yl]acetamide |
| 937 | ethyl ({6-[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′-yl}acetyl)amino]-2- |
| | azaspiro[3.3]hept-2-yl}sulfonyl)carbamate |
| 938 | 4-[(4-fluorobenzyl){[(3′R,4S)-3′-fluoro-5′-(1-methyl-1H-pyrazol-4-yl)-2,5- |
| | dioxo-2′,3′-dihydro-1H-spiro[imidazolidine-4,1′-inden]-1- |
| | yl]acetyl}amino]cyclohexanecarboxylic acid |
| 939 | N-(4-fluorobenzyl)-2-{(1R)-5-[(methylcarbamoyl)amino]-2′,4′-dioxo-2,3- |
| | dihydro-3′H-spiro[indene-1,5′-[1,3]oxazolidin]-3′-yl}-N-(3- |
| | {[(trifluoromethyl)sulfonyl]amino}cyclobutyl)acetamide |
| 940 | tert-butyl 3-[{[(1R)-5-bromo-2′,4′-dioxo-2,3-dihydro-3′H-spiro[indene-1,5′- |
| | [1,3]oxazolidin]-3′-yl]acetyl}(4-fluorobenzyl)amino]benzoate |
| 941 | tert-butyl 3-[(4-fluorobenzyl){[(1R)-5-(1-methyl-1H-pyrazol-4-yl)-2′,4′-dioxo- |
| | 2,3-dihydro-3′H-spiro[indene-1,5′-[1,3]oxazolidin]-3′- |
| | yl]acetyl}amino]benzoate |
| 942 | 3-[(4-fluorobenzyl){[(1R)-5-(1-methyl-1H-pyrazol-4-yl)-2′,4′-dioxo-2,3- |
| | dihydro-3′H-spiro[indene-1,5′-[1,3]oxazolidin]-3′-yl]acetyl}amino]benzoic |
| | acid |
| 943 | 2-[(4S)-5′-{1-[2-(dimethylamino)ethyl]-1H-pyrazol-4-yl}-2,5-dioxo-2′,3′- |
| | dihydro-1H-spiro[imidazolidine-4,1′-inden]-1-yl]-N-(4-fluorobenzyl)-N-[(2S)- |
| | 1,1,1-trifluoropropan-2-yl]acetamide |
| 944 | tert-butyl 4-({4-[(4S)-1-(2-{(4-fluorobenzyl)[(2S)-1,1,1-trifluoropropan-2- |
| | yl]amino}-2-oxoethyl)-2,5-dioxo-2′,3′-dihydrospiro[imidazolidine-4,1′-inden]- |
| | 5′-yl]-1H-pyrazol-1-yl}methyl)piperidine-1-carboxylate |
| 945 | 2-{(4S)-2,5-dioxo-5′-[1-(piperidin-4-ylmethyl)-1H-pyrazol-4-yl]-2′,3′-dihydro- |
| | 1H-spiro[imidazolidine-4,1′-inden]-1-yl}-N-(4-fluorobenzyl)-N-[(2S)-1,1,1- |
| | trifluoropropan-2-yl]acetamide |
| 946 | N-(4-fluorobenzyl)-2-{6-[(1-methyl-1H-pyrazol-4-yl)amino]-1,1-dioxido-2′,5′- |
| | dioxo-1′H-spiro[1-benzothiophene-3,4′-imidazolidin]-1′-yl}-N-[(2S)-1,1,1- |
| | trifluoropropan-2-yl]acetamide |
| 947 | N-(4-fluorobenzyl)-2-[(1S)-5-{1-[2-(methylamino)-2-oxoethyl]-1H-pyrazol-4- |
| | yl}-2′,4′-dioxo-2,3-dihydro-3′H-spiro[indene-1,5′-[1,3]oxazolidin]-3′-yl]-N- |
| | [(2S)-1,1,1-trifluoropropan-2-yl]acetamide |
| 948 | N-(azetidin-3-yl)-N-(4-fluorobenzyl)-2-{(1R)-5-[(methylcarbamoyl)amino]- |
| | 2′,4′-dioxo-2,3-dihydro-3′H-spiro[indene-1,5′-[1,3]oxazolidin]-3′-yl}acetamide |
| 949 | ethyl ({3-[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′- |
| | yl}acetyl)amino]azetidin-1-yl}sulfonyl)carbamate |
| 950 | 2-[(1S)-5-bromo-2′,4′-dioxo-2,3-dihydro-3′H-spiro[indene-1,5′- |
| | [1,3]oxazolidin]-3′-yl]-N-(4-fluorobenzyl)-N-[(2S)-1,1,1-trifluoropropan-2- |
| | yl]acetamide |
| 951 | N-(4-fluorobenzyl)-2-(6-methoxy-1,1-dioxido-2′,5′-dioxo-1′H-spiro[1- |
| | benzothiophene-3,4′-imidazolidin]-1′-yl)-N-[(2S)-1,1,1-trifluoropropan-2- |
| | yl]acetamide |
| 952 | N-(4-fluorobenzyl)-2-[(3S)-6-(1-methyl-1H-pyrazol-4-yl)-1,1-dioxido-2′,5′- |
| | dioxo-1′H-spiro[1-benzothiophene-3,4′-imidazolidin]-1′-yl]-N-[(2S)-1,1,1- |
| | trifluoropropan-2-yl]acetamide |
| 953 | N-(4-fluorobenzyl)-2-[(3R)-6-(1-methyl-1H-pyrazol-4-yl)-1,1-dioxido-2′,5′- |
| | dioxo-1′H-spiro[1-benzothiophene-3,4′-imidazolidin]-1′-yl]-N-[(2S)-1,1,1- |
| | trifluoropropan-2-yl]acetamide |
| 954 | N-(4-fluorobenzyl)-2-[(1S,3R)-3-hydroxy-5-(1-methyl-1H-pyrazol-4-yl)-2′,4′- |
| | dioxo-2,3-dihydro-3′H-spiro[indene-1,5′-[1,3]oxazolidin]-3′-yl]-N-[(2S)-1,1,1- |
| | trifluoropropan-2-yl]acetamide |
| 955 | N-benzyl-N-(1-cyclopropylethyl)-2-(2,5-dioxo-2′,3′-dihydro-1H- |
| | spiro[imidazolidine-4,1′-inden]-1-yl)acetamide |
| 956 | N-(1-cyclopropylethyl)-2-(2,5-dioxo-2′,3′-dihydro-1H-spiro[imidazolidine- |
| | 4,1′-inden]-1-yl)-N-(1H-imidazol-4-ylmethyl)acetamide |
| 957 | N-[(1S)-3′-(2-{[(1S)-1-cyclopropylethyl](4-fluorobenzyl)amino}-2-oxoethyl)- |
| | 2′,4′-dioxo-2,3-dihydrospiro[indene-1,5′-[1,3]oxazolidin]-5-yl]-D-valinamide |
| 958 | N-benzyl-N-(1-cyclopropylethyl)-2-(2,2-dimethyl-2′,5′-dioxo-1′H-spiro[1- |
| | benzofuran-3,4′-imidazolidin]-1′-yl)acetamide |
| 959 | N-benzyl-N-(1-cyclopropylethyl)-2-(1′,3-dimethyl-2,2′,5-trioxo-1′,2′-dihydro- |
| | 1H-spiro[imidazolidine-4,3′-indol]-1-yl)acetamide |
| 960 | N-benzyl-N-(1-cyclopropylethyl)-2-(2,5-dioxo-2′,3′-dihydro-1H- |
| | spiro[imidazolidine-4,1′-inden]-1-yl)-2-methylpropanamide |
| 961 | N-benzyl-N-(1-cyclopropylethyl)-2-[2,5-dioxo-3-(prop-2-en-1-yl)-2′,3′- |
| | dihydro-1H-spiro[imidazolidine-4,1′-inden]-1-yl]acetamide |
| 962 | N-(1-cyclopropylethyl)-2-(2,5-dioxo-2′,3′-dihydro-1H-spiro[imidazolidine- |
| | 4,1′-inden]-1-yl)-N-(2,2,2-trifluoroethyl)acetamide |
| 963 | N-(1-cyclopropylethyl)-2-(2,5-dioxo-2′,3′-dihydro-1H-spiro[imidazolidine- |
| | 4,1′-inden]-1-yl)-N-[3-(trifluoromethoxy)benzyl]acetamide |
| 964 | N-(but-2-yn-1-yl)-N-(1-cyclopropylethyl)-2-(2,5-dioxo-2′,3′-dihydro-1H- |
| | spiro[imidazolidine-4,1′-inden]-1-yl)acetamide |
| 965 | N-(1-cyclopropylethyl)-N-{[6-(difluoromethoxy)naphthalen-2-yl]methyl}-2- |
| | (2,5-dioxo-2′,3′-dihydro-1H-spiro[imidazolidine-4,1′-inden]-1-yl)acetamide |
| 966 | N-benzyl-N-(1-cyclopropylethyl)-2-[5′-(methylsulfonyl)-2,5-dioxo-2′,3′- |
| | dihydro-1H-spiro[imidazolidine-4,1′-inden]-1-yl]acetamide |
| 967 | N-benzyl-N-(1-cyclobutylethyl)-2-(7′-methoxy-2,5-dioxo-2′,3′-dihydro-1H- |
| | spiro[imidazolidine-4,1′-inden]-1-yl)acetamide |
| 968 | N-benzyl-N-(1-cyclopropylethyl)-2-(1-methyl-2′,5′-dioxo-5,6-dihydro-1H,1′H- |
| | spiro[cyclopenta[c]pyrazole-4,4′-imidazolidin]-1′-yl)acetamide |
| 969 | N-benzyl-N-(1-cyclopropylethyl)-2-[5′-(hydroxymethyl)-3-methyl-2,5-dioxo- |
| | 2′,3′-dihydro-1H-spiro[imidazolidine-4,1′-inden]-1-yl]acetamide |
| 970 | N-[(1S)-1-cyclobutylethyl]-2-[(4R)-2,5-dioxo-2′,3′-dihydro-1H- |
| | spiro[imidazolidine-4,1′-inden]-1-yl]-N-(prop-2-yn-1-yl)acetamide |
| 971 | N³-[(1S)-1-cyclobutylethyl]-N³-{[(4R)-2,5-dioxo-2′,3′-dihydro-1H- |
| | spiro[imidazolidine-4,1′-inden]-1-yl]acetyl}-beta-alaninamide |
| 972 | N-(biphenyl-4-ylmethyl)-N-[(1S)-1-cyclobutylethyl]-2-[(4R)-2,5-dioxo-2′,3′- |
| | dihydro-1H-spiro[imidazolidine-4,1′-inden]-1-yl]acetamide |
| 973 | N²-[(1S)-1-cyclobutylethyl]-N²-{[(4R)-2,5-dioxo-2′,3′-dihydro-1H- |
| | spiro[imidazolidine-4,1′-inden]-1-yl]acetyl}glycinamide |
| 974 | N-[(1S)-1-cyclobutylethyl]-2-[(4R)-2,5-dioxo-2′,3′-dihydro-1H- |
| | spiro[imidazolidine-4,1′-inden]-1-yl]-N-[(1-methyl-2-oxo-1,2-dihydropyridin- |
| | 4-yl)methyl]acetamide |
| 975 | N-benzyl-N-[(1R)-1-cyclopropylethyl]-2-[(4R)-2,5-dioxo-2′,3′-dihydro-1H- |
| | spiro[imidazolidine-4,1′-inden]-1-yl]acetamide |
| 976 | N-benzyl-N-[(1S)-1-cyclopropylethyl]-2-[(4S)-2,5-dioxo-2′,3′-dihydro-1H- |
| | spiro[imidazolidine-4,1′-inden]-1-yl]acetamide |
| 977 | N-benzyl-N-[(1S)-1-cyclopropylethyl]-2-[(4R)-2,5-dioxo-2′,3′-dihydro-1H- |
| | spiro[imidazolidine-4,1′-inden]-1-yl]acetamide |
| 978 | N-benzyl-N-[(1R)-1-cyclopropylethyl]-2-[(4S)-2,5-dioxo-2′,3′-dihydro-1H- |
| | spiro[imidazolidine-4,1′-inden]-1-yl]acetamide |
| 979 | N-[4-(acetylamino)benzyl]-N-[(1S)-1-cyclobutylethyl]-2-[(4R)-2,5-dioxo-2′,3′- |
| | dihydro-1H-spiro[imidazolidine-4,1′-inden]-1-yl]acetamide |
| 980 | N-[(1S)-1-cyclobutylethyl]-2-[(4R)-2,5-dioxo-2′,3′-dihydro-1H- |
| | spiro[imidazolidine-4,1′-inden]-1-yl]-N-(4-ethynylbenzyl)acetamide |
| 981 | N-[(1S)-1-cyclobutylethyl]-2-[(4R)-2,5-dioxo-2′,3′-dihydro-1H- |
| | spiro[imidazolidine-4,1′-inden]-1-yl]-N-({1-[2-(methylamino)-2-oxoethyl]- |
| | 1H-1,2,3-triazol-4-yl}methyl)acetamide |
| 982 | 2-{4-[([(1S)-1-cyclobutylethyl]{[(4R)-2,5-dioxo-2′,3′-dihydro-1H- |
| | spiro[imidazolidine-4,1′-inden]-1-yl]acetyl}amino)methyl]-1H-1,2,3-triazol-1- |
| | yl}-N,N-dimethylacetamide |
| 983 | N³-[(1S)-1-cyclobutylethyl]-N³-{[(4R)-2,5-dioxo-2′,3′-dihydro-1H- |
| | spiro[imidazolidine-4,1′-inden]-1-yl]acetyl}-N-methyl-beta-alaninamide |
| 984 | N-benzyl-N-(1-cyclopropylethyl)-2-[5′-(6-hydroxypyridin-3-yl)-2,5-dioxo- |
| | 2′,3′-dihydro-1H-spiro[imidazolidine-4,1′-inden]-1-yl]acetamide |
| 985 | N-benzyl-N-[(1S)-1-cyclopropylethyl]-2-[5′-(formylamino)-2,5-dioxo-2′,3′- |
| | dihydro-1H-spiro[imidazolidine-4,1′-inden]-1-yl]acetamide |
| 986 | N-benzyl-N-[(1S)-1-cyclopropylethyl]-2-[2′,4′-dioxo-5-(2H-tetrazol-5-yl)-2,3- |
| | dihydro-3′H-spiro[indene-1,5′-[1,3]oxazolidin]-3′-yl]acetamide |
| 987 | 2,2′-({[3′-(2-{benzyl[(1S)-1-cyclopropylethyl]amino}-2-oxoethyl)-2′,4′-dioxo- |
| | 2,3-dihydrospiro[indene-1,5′-[1,3]oxazolidin]-5-yl]carbonyl}imino)diacetic |
| | acid |
| 988 | N-benzyl-2-[(4R)-5′-bromo-2,5-dioxo-2′,3′-dihydro-1H-spiro[imidazolidine- |
| | 4,1′-inden]-1-yl]-N-[(1S)-1-cyclopropylethyl]acetamide |
| 989 | N-(4-fluorobenzyl)-2-[(1S,3R)-3-hydroxy-5-{1-[2-(methylamino)-2-oxoethyl]- |
| | 1H-pyrazol-4-yl}-2′,4′-dioxo-2,3-dihydro-3′H-spiro[indene-1,5′- |
| | [1,3]oxazolidin]-3′-yl]-N-[(2S)-1,1,1-trifluoropropan-2-yl]acetamide |
| 990 | 2-{(1S,3R)-5-[1-(2-amino-2-oxoethyl)-1H-pyrazol-4-yl]-3-hydroxy-2′,4′- |
| | dioxo-2,3-dihydro-3′H-spiro[indene-1,5′-[1,3]oxazolidin]-3′-yl}-N-(4- |
| | fluorobenzyl)-N-[(2S)-1,1,1-trifluoropropan-2-yl]acetamide |
| 991 | N-(4-fluorobenzyl)-2-[(1S)-5-(1-methyl-1H-pyrazol-4-yl)-2′,4′-dioxo-2,3- |
| | dihydro-3′H-spiro[indene-1,5′-[1,3]oxazolidin]-3′-yl]-N-[(2S)-1,1,1- |
| | trifluoropropan-2-yl]acetamide |
| 992 | 2-[1,1-dioxido-2′,5′-dioxo-6-(pyridin-3-yl)-1′H-spiro[1-benzothiophene-3,4′- |
| | imidazolidin]-1′-yl]-N-(4-fluorobenzyl)-N-[(2S)-1,1,1-trifluoropropan-2- |
| | yl]acetamide |
| 993 | 2-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′-yl}acetyl)amino]benzoic |
| | acid |
| 994 | methyl 3-[(4-fluorobenzyl)({(1R)-5-[(methylcarbamoyl)amino]-2′,4′-dioxo- |
| | 2,3-dihydro-3′H-spiro[indene-1,5′-[1,3]oxazolidin]-3′- |
| | yl}acetyl)amino]butanoate |
| 995 | 3-[(4-fluorobenzyl)({(1R)-5-[(methylcarbamoyl)amino]-2′,4′-dioxo-2,3- |
| | dihydro-3′H-spiro[indene-1,5′-[1,3]oxazolidin]-3′-yl}acetyl)amino]butanoic |
| | acid |
| 996 | 2-{(1S)-5-[1-(2-amino-2-oxoethyl)-1H-pyrazol-4-yl]-2′,4′-dioxo-2,3-dihydro- |
| | 3′H-spiro[indene-1,5′-[1,3]oxazolidin]-3′-yl}-N-(4-fluorobenzyl)-N-[(2S)- |
| | 1,1,1-trifluoropropan-2-yl]acetamide |
| 997 | N-(4-fluorobenzyl)-2-[(3′S,4S)-3′-fluoro-5′-{1-[2-(methylamino)-2-oxoethyl]- |
| | 1H-pyrazol-4-yl}-2,5-dioxo-2′,3′-dihydro-1H-spiro[imidazolidine-4,1′-inden]- |
| | 1-yl]-N-[(2S)-1,1,1-trifluoropropan-2-yl]acetamide |
| 998 | N-(4-fluorobenzyl)-2-[(1R,3R)-3-hydroxy-5-(1-methyl-1H-pyrazol-4-yl)-2′,4′- |
| | dioxo-2,3-dihydro-3′H-spiro[indene-1,5′-[1,3]oxazolidin]-3′-yl]-N-[(2S)-1,1,1- |
| | trifluoropropan-2-yl]acetamide |
| 999 | N-(4-fluorobenzyl)-2-[(1R,3R)-3-hydroxy-5-{1-[2-(methylamino)-2- |
| | oxoethyl]-1H-pyrazol-4-yl}-2′,4′-dioxo-2,3-dihydro-3′H-spiro[indene-1,5′- |
| | [1,3]oxazolidin]-3′-yl]-N-[(2S)-1,1,1-trifluoropropan-2-yl]acetamide |
| 1000 | N-(4-fluorobenzyl)-2-[(1S,3S)-3-hydroxy-5-(1-methyl-1H-pyrazol-4-yl)-2′,4′- |
| | dioxo-2,3-dihydro-3′H-spiro[indene-1,5′-[1,3]oxazolidin]-3′-yl]-N-[(2S)-1,1,1- |
| | trifluoropropan-2-yl]acetamide |
| 1001 | 2-[(1S)-5-{1-[2-(dimethylamino)-2-oxoethyl]-1H-pyrazol-4-yl}-2′,4′-dioxo- |
| | 2,3-dihydro-3′H-spiro[indene-1,5′-[1,3]oxazolidin]-3′-yl]-N-(4-fluorobenzyl)- |
| | N-[(2S)-1,1,1-trifluoropropan-2-yl]acetamide |
| 1002 | 2-[(1S,3R)-5-bromo-3-fluoro-2′,4′-dioxo-2,3-dihydro-3′H-spiro[indene-1,5′- |
| | [1,3]oxazolidin]-3′-yl]-N-(4-fluorobenzyl)-N-[(2S)-1,1,1-trifluoropropan-2- |
| | yl]acetamide |
| 1003 | 2-[(1R)-2′,4′-dioxo-5-(pyridin-4-yl)-2,3-dihydro-3′H-spiro[indene-1,5′- |
| | [1,3]oxazolidin]-3′-yl]-N-(4-fluorobenzyl)-N-[(2S)-1,1,1-trifluoropropan-2- |
| | yl]acetamide |
| 1004 | 2-[(1S)-2′,4′-dioxo-5-(pyridin-4-yl)-2,3-dihydro-3′H-spiro[indene-1,5′- |
| | [1,3]oxazolidin]-3′-yl]-N-(4-fluorobenzyl)-N-[(2S)-1,1,1-trifluoropropan-2- |
| | yl]acetamide |
| 1005 | 2-[(1R)-2′,4′-dioxo-5-(pyridin-3-yl)-2,3-dihydro-3′H-spiro[indene-1,5′- |
| | [1,3]oxazolidin]-3′-yl]-N-(4-fluorobenzyl)-N-[(2S)-1,1,1-trifluoropropan-2- |
| | yl]acetamide |
| 1006 | 2-[(1S)-2′,4′-dioxo-5-(pyridin-3-yl)-2,3-dihydro-3′H-spiro[indene-1,5′- |
| | [1,3]oxazolidin]-3′-yl]-N-(4-fluorobenzyl)-N-[(2S)-1,1,1-trifluoropropan-2- |
| | yl]acetamide |
| 1007 | 2-[(1R)-2′,4′-dioxo-5-(6-oxo-1,6-dihydropyridin-3-yl)-2,3-dihydro-3′H- |
| | spiro[indene-1,5′-[1,3]oxazolidin]-3′-yl]-N-(4-fluorobenzyl)-N-[(2S)-1,1,1- |
| | trifluoropropan-2-yl]acetamide |
| 1008 | 2-[(1S)-2′,4′-dioxo-5-(6-oxo-1,6-dihydropyridin-3-yl)-2,3-dihydro-3′H- |
| | spiro[indene-1,5′-[1,3]oxazolidin]-3′-yl]-N-(4-fluorobenzyl)-N-[(2S)-1,1,1- |
| | trifluoropropan-2-yl]acetamide |
| 1009 | 2-[(1R)-2′,4′-dioxo-5-phenyl-2,3-dihydro-3′H-spiro[indene-1,5′- |
| | [1,3]oxazolidin]-3′-yl]-N-(4-fluorobenzyl)-N-[(2S)-1,1,1-trifluoropropan-2- |
| | yl]acetamide |
| 1010 | 2-[(1S)-2′,4′-dioxo-5-phenyl-2,3-dihydro-3′H-spiro[indene-1,5′- |
| | [1,3]oxazolidin]-3′-yl]-N-(4-fluorobenzyl)-N-[(2S)-1,1,1-trifluoropropan-2- |
| | yl]acetamide |
| 1011 | N-cyclohexyl-N-(4-fluorobenzyl)-2-[(1R)-5-(1-methyl-1H-pyrazol-4-yl)-2′,4′- |
| | dioxo-2,3-dihydro-3′H-spiro[indene-1,5′-[1,3]oxazolidin]-3′-yl]acetamide |
| 1012 | N-(4-fluorobenzyl)-2-[(1R)-5-(1-methyl-1H-pyrazol-4-yl)-2′,4′-dioxo-2,3- |
| | dihydro-3′H-spiro[indene-1,5′-[1,3]oxazolidin]-3′-yl]-N-(tetrahydro-2H-pyran- |
| | 4-yl)acetamide |
| 1013 | 3-[(4-fluorobenzyl){[(1R)-5-(1-methyl-1H-pyrazol-4-yl)-2′,4′-dioxo-2,3- |
| | dihydro-3′H-spiro[indene-1,5′-[1,3]oxazolidin]-3′- |
| | yl]acetyl}amino]cyclobutanecarboxylic acid |
| 1014 | 4-[(4-fluorobenzyl){[(1R)-5-(1-methyl-1H-pyrazol-4-yl)-2′,4′-dioxo-2,3- |
| | dihydro-3′H-spiro[indene-1,5′-[1,3]oxazolidin]-3′- |
| | yl]acetyl}amino]cyclohexanecarboxylic acid |
| 1015 | N-(4-fluorobenzyl)-2-[(1R)-5-(1-methyl-1H-pyrazol-4-yl)-2′,4′-dioxo-2,3- |
| | dihydro-3′H-spiro[indene-1,5′-[1,3]oxazolidin]-3′-yl]-N-{trans-4- |
| | [(methylsulfonyl)amino]cyclohexyl}acetamide |
| 1016 | N-(4-fluorobenzyl)-2-[(1R)-5-(1-methyl-1H-pyrazol-4-yl)-2′,4′-dioxo-2,3- |
| | dihydro-3′H-spiro[indene-1,5′-[1,3]oxazolidin]-3′-yl]-N-(3- |
| | {[(trifluoromethyl)sulfonyl]amino}cyclobutyl)acetamide |
| 1017 | N-(4-fluorobenzyl)-2-[(1R)-5-(1-methyl-1H-pyrazol-4-yl)-2′,4′-dioxo-2,3- |
| | dihydro-3′H-spiro[indene-1,5′-[1,3]oxazolidin]-3′-yl]-N-[1- |
| | (methylsulfonyl)piperidin-4-yl]acetamide |
| 1018 | 2-[(1R,3S)-5-bromo-3-hydroxy-2′,4′-dioxo-2,3-dihydro-3′H-spiro[indene-1,5′- |
| | [1,3]oxazolidin]-3′-yl]-N-(4-fluorobenzyl)-N-[(2S)-1,1,1-trifluoropropan-2- |
| | yl]acetamide |
| 1019 | N-(4-fluorobenzyl)-2-[(1R,3S)-3-hydroxy-5-(1-methyl-1H-pyrazol-4-yl)-2′,4′- |
| | dioxo-2,3-dihydro-3′H-spiro[indene-1,5′-[1,3]oxazolidin]-3′-yl]-N-[(2S)-1,1,1- |
| | trifluoropropan-2-yl]acetamide |
| 1020 | 2,2′-({3-[(4-fluorobenzyl){[(1R)-5-(1-methyl-1H-pyrazol-4-yl)-2′,4′-dioxo-2,3- |
| | dihydro-3′H-spiro[indene-1,5′-[1,3]oxazolidin]-3′- |
| | yl]acetyl}amino]cyclobutyl}imino)diacetic acid |
| 1021 | N-(4-fluorobenzyl)-2-[(1S,3R)-3-fluoro-5-{1-[2-(methylamino)-2-oxoethyl]- |
| | 1H-pyrazol-4-yl}-2′,4′-dioxo-2,3-dihydro-3′H-spiro[indene-1,5′- |
| | [1,3]oxazolidin]-3′-yl]-N-[(2S)-1,1,1-trifluoropropan-2-yl]acetamide |
| 1022 | N-(4-fluorobenzyl)-2-[(1R)-5-(1,2-oxazol-4-yl)-2′,4′-dioxo-2,3-dihydro-3′H- |
| | spiro[indene-1,5′-[1,3]oxazolidin]-3′-yl]-N-[(2S)-1,1,1-trifluoropropan-2- |
| | yl]acetamide |
| 1023 | N-(4-fluorobenzyl)-2-[(1S)-5-(1,2-oxazol-4-yl)-2′,4′-dioxo-2,3-dihydro-3′H- |
| | spiro[indene-1,5′-[1,3]oxazolidin]-3′-yl]-N-[(2S)-1,1,1-trifluoropropan-2- |
| | yl]acetamide |
| 1024 | N-(4-fluorobenzyl)-2-[(1R,3S)-3-hydroxy-5-{1-[2-(methylamino)-2-oxoethyl]- |
| | 1H-pyrazol-4-yl}-2′,4′-dioxo-2,3-dihydro-3′H-spiro[indene-1,5′- |
| | [1,3]oxazolidin]-3′-yl]-N-[(2S)-1,1,1-trifluoropropan-2-yl]acetamide |
| 1025 | N-(4-fluorobenzyl)-2-{(1S,3R)-3-fluoro-5-[(methylcarbamoyl)amino]-2′,4′- |
| | dioxo-2,3-dihydro-3′H-spiro[indene-1,5′-[1,3]oxazolidin]-3′-yl}-N-[(2S)-1,1,1- |
| | trifluoropropan-2-yl]acetamide |
| 1026 | (4-{[(5′-bromo-6′-fluoro-2,5-dioxo-2′,3′-dihydro-1H-spiro[imidazolidine-4,1′- |
| | inden]-1-yl)acetyl](4-fluorobenzyl)amino}cyclohexylidene)acetic acid |
| 1027 | {4-[(4-fluorobenzyl){[6′-fluoro-5′-(1-methyl-1H-pyrazol-4-yl)-2,5-dioxo-2′,3′- |
| | dihydro-1H-spiro[imidazolidine-4,1′-inden]-1- |
| | yl]acetyl}amino]cyclohexylidene}acetic acid |
| 1028 | tert-butyl (4-{[(5′-bromo-6′-fluoro-2,5-dioxo-2′,3′-dihydro-1H- |
| | spiro[imidazolidine-4,1′-inden]-1-yl)acetyl](4- |
| | fluorobenzyl)amino}cyclohexylidene)acetate |
| 1029 | 2-[(1R,3S)-5-bromo-3-fluoro-2′,4′-dioxo-2,3-dihydro-3′H-spiro[indene-1,5′- |
| | [1,3]oxazolidin]-3′-yl]-N-(4-fluorobenzyl)-N-[(2S)-1,1,1-trifluoropropan-2- |
| | yl]acetamide |
| 1030 | N-(4-fluorobenzyl)-2-[(1R,3S)-3-fluoro-5-{1-[2-(methylamino)-2-oxoethyl]- |
| | 1H-pyrazol-4-yl}-2′,4′-dioxo-2,3-dihydro-3′H-spiro[indene-1,5′- |
| | [1,3]oxazolidin]-3′-yl]-N-[(2S)-1,1,1-trifluoropropan-2-yl]acetamide |
| 1031 | {4-[(4-fluorobenzyl){[(1S,3R)-3-fluoro-5-(1-methyl-1H-pyrazol-4-yl)-2′,4′- |
| | dioxo-2,3-dihydro-3′H-spiro[indene-1,5′-[1,3]oxazolidin]-3′- |
| | yl]acetyl}amino]cyclohexylidene}acetic acid |
| 1032 | N-(4-fluorobenzyl)-2-[(1R,3R)-3-fluoro-5-(1-methyl-1H-pyrazol-4-yl)-2′,4′- |
| | dioxo-2,3-dihydro-3′H-spiro[indene-1,5′-[1,3]oxazolidin]-3′-yl]-N-[(2S)-1,1,1- |
| | trifluoropropan-2-yl]acetamide |
| 1033 | N-(4-fluorobenzyl)-2-[(1R,3R)-3-fluoro-5-{1-[2-(methylamino)-2-oxoethyl]- |
| | 1H-pyrazol-4-yl}-2′,4′-dioxo-2,3-dihydro-3′H-spiro[indene-1,5′- |
| | [1,3]oxazolidin]-3′-yl]-N-[(2S)-1,1,1-trifluoropropan-2-yl]acetamide |

It is to be understood that any embodiment of the compounds of structure (I), as set forth above, and any specific substituent or value set forth herein for R¹, R^{2a}, R^{2b}, R^{3a}, R^{3b}, R^{4a}, R^{4b}, R^{5a}, R^{5b}, R⁶, R⁷, A, B, Q, W, x, y and/or z in the compounds of structure (I), as set forth above, may be independently combined with other embodiments and/or substituents and/or values of the above variables of compounds of structure (I) to form embodiments of the inventions not specifically set forth above. In addition, in the event that a list of choices is listed for any particular R group or other variable in a particular embodiment and/or claim, it is to be understood that each individual choice may be deleted from the particular embodiment and/or claim and that the remaining list of choices will be considered to be within the scope of the invention.

It is to be understood that in the present description, combinations of substituents and/or variables of the depicted formulae are permissible only if such contributions result in stable compounds.

The compounds of the present invention (i.e., compounds of Structure I, II and III) may contain one or more asymmetric centers. Compounds as described herein include all stereoisomers. Accordingly, the compounds include racemic mixtures, enantiomers and diastereomers of any of the compounds described herein. Tautomers of any of the compounds described herein are also included within the scope of the invention.

Accordingly, in some embodiments the compounds are mixtures of different enantiomers (e.g., R and S) or different diastereomers. In other embodiments, the compounds are pure (or enriched) enantiomers or diastereomers. For purpose of clarity, the chiral carbons are not always depicted in the compounds; however, the present invention includes all stereoisomers (pure and mixtures) of all compounds of Structure I, II and III.

By way of example, certain embodiments of the compounds of Structure I, contain at least one stereocenter. For example, in some embodiments the compounds have one of the following structures (Ia) or (Ib):

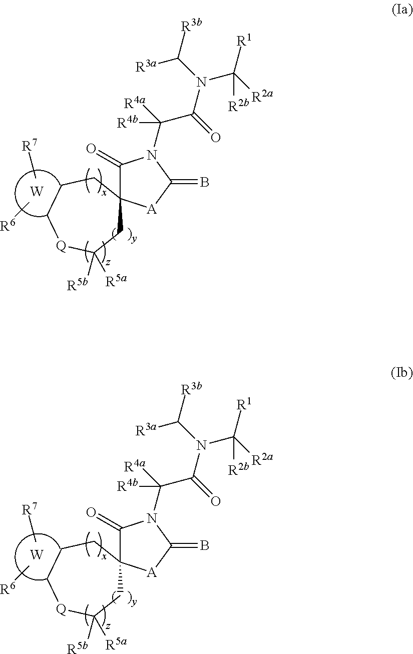

In other embodiments, the compounds have one of the following structures (Ic) or (Id):

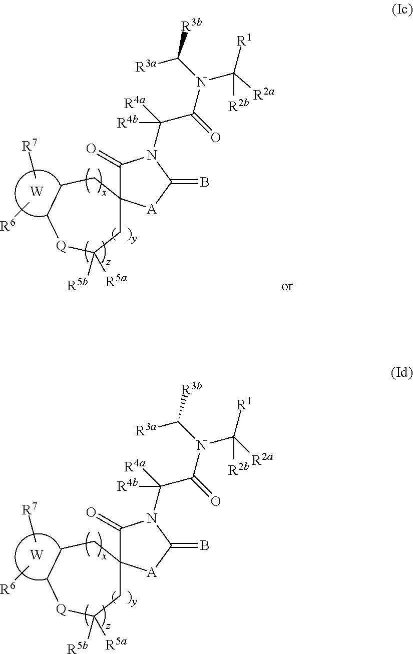

In still other embodiments, the compounds comprise at least two stereocenters. For example, in some embodiments the compounds have one of the following structures (Ie), (If), (Ig) or (Ih):

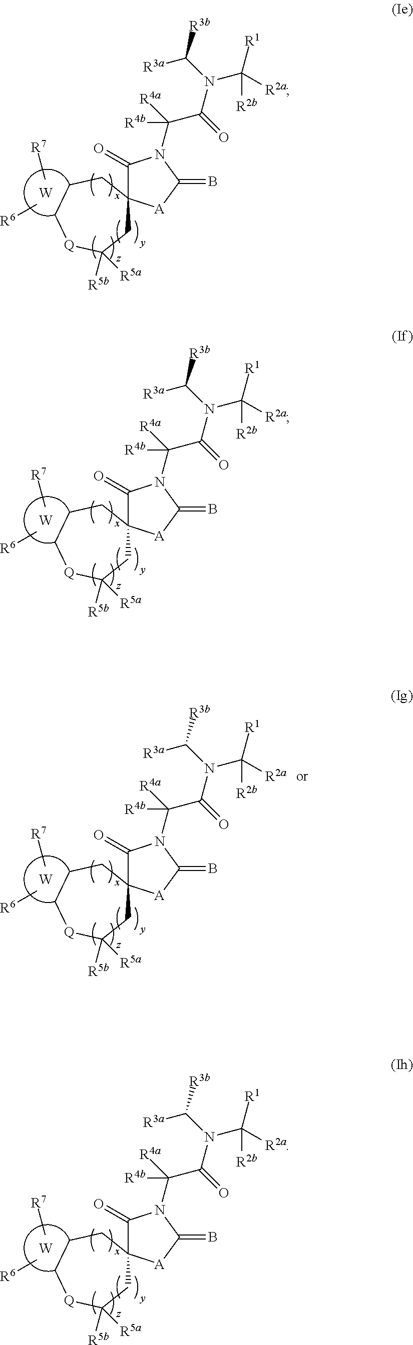

In an analagous fashion, the invention also includes all possible stereoisomers of all compounds of Structure II and III, including the compounds provided in Table 1. One of ordinary skill in the art will readily understand how to derive all possible stereoisomers, especially in reference to the above exemplary compounds of Structure I.

General Reaction Scheme I illustrates an exemplary method of making compounds of this invention, i.e., compound of structure (I). Variations of General Reaction Scheme I and alternative methods of making the compounds of the invention are described in more detail in the Examples.

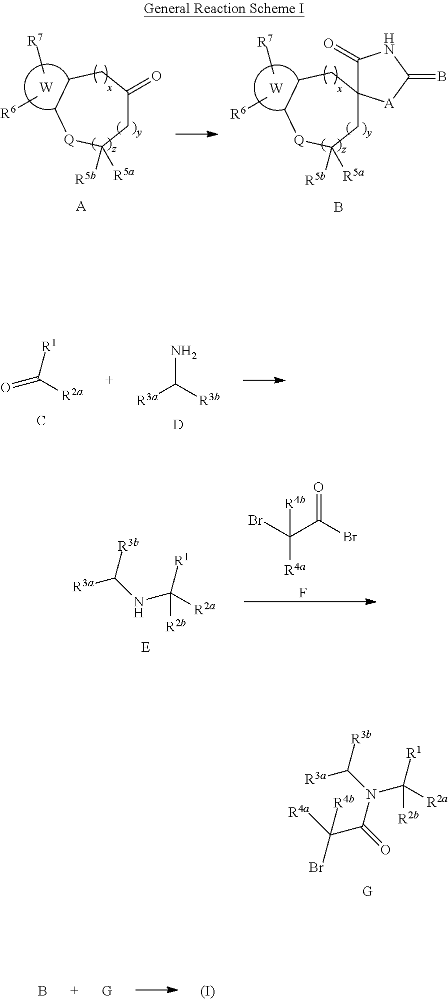

Referring to General Reaction Scheme I, compounds of structure A can be purchased or prepared according to methods well-known to those of ordinary skill in the art. Examples of such methods are provided in the Examples. Reaction of A with an appropriate reagent results in B. In embodiments where A is —NH—, appropriate reagents include ammonium carbonate and sodium cyanide. Other embodiments wherein A is —O— are prepared by reaction of A with trimethylsilyl nitrile, followed by treatment with HCl and triphosgene. Other various methods for preparing different embodiments of compounds of structure B are well-known to those of ordinary skill in the art.

In a parallel synthetic pathway, compounds of structure C and D are reacted under reductive amination conditions to yield compounds of structure E. Compounds of structures C and D can be purchased or prepared according to methods known in the art. Compounds wherein R^{2b} is C₁-C₆ alkyl, can be prepared using appropriate alkylation procedures known in the art. Furthermore, enantiomerically pure or racemic compound of structure D can be used depending on the desired product. Reaction of E with an appropriate acylating reagent, such as F, results in compounds of structure G. Reaction of G with B under appropriate conditions (e.g., basic conditions) results in compounds of structure I.

It is understood that one skilled in the art may be able to make these compounds by similar methods or by combining other methods known to one skilled in the art. It is also understood that one skilled in the art would be able to make, in a similar manner as described below, other compounds of structure (I) not specifically illustrated below by using the appropriate starting components and modifying the parameters of the synthesis as needed. For example, in various embodiments the substituents R⁶ and/or R⁷ are installed after the spiro cyclic ring structure is assembled) In these embodiments, it may be useful to include a bromide or other suitable leaving group in the compound of structure A so that the compounds can be further functionalized. Single stereoisomers at the spiro cyclic ring juncture can be prepared by chiral (e.g., enzymatic) hydrolysis of compounds of structure B. Recyclization and preparation of compounds of structure I using similar conditions is then possible. Such methods are provided in more detail in the Examples.

In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described in this invention.

It will also be appreciated by those skilled in the art that in the processes described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T.W. and P.G.M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this invention may not possess pharmacological activity as such, they may be administered to a mammal and thereafter metabolized in the body to form compounds of the invention which are pharmacologically active. Such derivatives may therefore be described as “prodrugs”. All prodrugs of compounds of this invention are included within the scope of the invention.

It should be appreciated that compounds of the invention, and more specifically, compounds in the experimentals, may be isolated as salts in situ during final isolation and purification. Furthermore, all compounds of the invention which exist in free base or acid form can be converted to their pharmaceutically acceptable salts by treatment with the appropriate inorganic or organic base or acid by methods known to one skilled in the art. Salts of the compounds of the invention can be converted to their free base or acid form by standard techniques. The tested form of the compound may or may not be indicated in the experimentals.

### II. Pharmaceutical Compositions and Administration

For the purposes of administration, the compounds of the present invention may be administered as a raw chemical or may be formulated as pharmaceutical compositions. In certain embodiments, the present invention is directed to a pharmaceutical composition comprising a pharmaceutically acceptable carrier or excipient and a compound having the following structure (I):

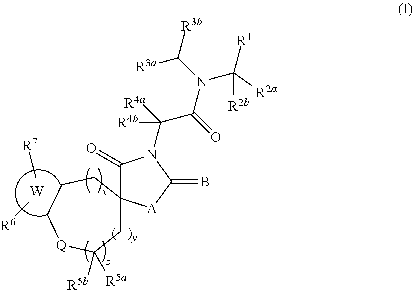

or a stereoisomer, tautomer or pharmaceutically acceptable salt thereof, wherein:
A is —NR⁸—, —O—, or —S—;
B is O or NH;
Q is —CHR¹⁰—, —O—, —S(O)ₘ—, —NR⁹— or absent;
W is an aryl or heteroaryl ring;
R¹ is carbocyclyl or heterocyclyl;
R^{2a} and R^{2b} are each independently H, D, or C₁-C₆ alkyl;
R^{3a} is cycloalkyl or heterocyclyl and R^{3b} is H or C₁-C₆ alkyl or cycloalkyl; or R^{3a} and R^{3b} are each independently C₁-C₆ alkyl; or R^{3a} and R^{3b} taken together form a heterocycle; or R^{3a} is C₁-C₆ alkyl and R^{3b} is C₁-C₆ substituted alkyl;
R^{4a} and R^{4b} are each independently H, D or C₁-C₆alkyl;
R^{5a} and R^{5b} are each independently H, D, —OH, —CO₂H, —CN, —CO₂NH₂, —C₁-C₆ alkyl or alkoxy; or R^{5a} and R^{5b} taken together form oxo or ═N—OR¹¹;
R⁶ and R⁷ are each independently H, halo, —OH, —CN, —CO₂H, C₁-C₆ alkyl, C₁-C₆ alkynyl, alkoxy, haloalkoxy, alkoxyalkyl, haloalkoxyalkyl, hydroxylalkyl, hydroxylalkynyl, cycloalkyl, heterocyclyl, heterocyclylalkyl, heterocyclyloxy, —B(R¹¹)(R¹³), —S(O)ₘR², —NHR¹², —C(═O)NHR¹², —NHC(═O)R¹², —NHC(═O)OR¹², —NHC(═O)C(═O)NHR¹², —NHC(═O)C(═O)OR¹², —NHC(═O)NHR¹², —NHC(═O)NR¹²C(═O)NHR¹² or —NHS(O)ₘR¹²;
R⁸ and R⁹ are each independently H or C₁-C₆alkyl;
R¹⁰ is H, OH, —CN, —CO₂R¹², —C(═O)NHR¹³, C₁-C₆ alkyl or alkoxy;
R¹¹ and R¹³ are each independently H, OH or C₁-C₆alkyl;
R¹² is, at each occurrence, independently H, C₁-C₆ alkyl, cycloalkyl, heterocyclyl or heterocyclyalkyl;
m is, at each occurrence, independently 0, 1 or 2; and
x, y and z are each independently 0 or 1, wherein x, y and z are selected such that the sum of x+y+z is 1 or 2.

In more specific embodiments, the compound is a compound as described in the foregoing section entitled “compounds.”

The compound of structure (I) is present in the composition in an amount which is effective to treat a particular disease or condition of interest—that is, in an amount sufficient to inhibit HAT activity, and preferably with acceptable toxicity to the patient. HAT activity of compounds of structure (I) can be determined by one skilled in the art, for example, as described in the Examples below. Appropriate concentrations and dosages can be readily determined by one skilled in the art.

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition of the invention may be intended for rectal administration, in the form, for example, of a suppository, which will melt in the rectum and release the drug. The composition for rectal administration may contain an oleaginous base as a suitable non irritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Compounds of the invention, or pharmaceutically acceptable derivatives thereof, may also be administered simultaneously with, prior to, or after administration of one or more other therapeutic agents. Such combination therapy includes administration of a single pharmaceutical dosage formulation which contains a compound of the invention and one or more additional active agents, as well as administration of the compound of the invention and each active agent in its own separate pharmaceutical dosage formulation. For example, a compound of the invention and the other active agent can be administered to the patient together in a single oral dosage composition such as a tablet or capsule, or each agent administered in separate oral dosage formulations. Where separate dosage formulations are used, the compounds of the invention and one or more additional active agents can be administered at essentially the same time, i.e., concurrently, or at separately staggered times, i.e., sequentially; combination therapy is understood to include all these regimens.

### III. Methods

### Diseases, Disorders, and Conditions

The compounds for inhibiting the activity of p300/CBP disclosed herein can be useful in analyzing p300/CBP signaling activity in model systems and for preventing, treating, or ameliorating of a symptom associated with a disease, disorder, or pathological condition involving p300/CBP, preferably one afflicting humans. A compound which inhibits the activity of p300/CBP will be useful in preventing, treating, ameliorating, or reducing the symptoms or progression of cancer, cardiac disease, metabolic disease, fibrotic disease, inflammatory disease, or viral infections. The present invention provides methods for inhibiting p300/CBP comprising administering the compounds described herein in a therapeutically effective amount to a subject in need thereof. A subject may be a human, non-human primate, rodent, canine, feline, ungulate, bovine, equine, or other species.

A wide variety of cancers, including solid tumors and leukemias are amenable to the compositions and methods disclosed herein. Types of cancer that may be treated include, but are not limited to: adenocarcinoma of the breast, prostate, and colon; all forms of bronchogenic carcinoma of the lung; myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell). Additional types of cancers that may be treated include: histiocytic disorders; leukemia; histiocytosis malignant; Hodgkin's disease; immunoproliferative small; non-Hodgkin's lymphoma; plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin. The types of cancers that may be treated also include, but are not limited to, angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

In a particular embodiment, the present disclosure provides for methods of treating colon cancer, gastric cancer, thyroid cancer, lung cancer, leukemia, pancreatic cancer, melanoma, multiple melanoma, brain cancer, CNS cancer, renal cancer, prostate cancer, ovarian cancer, leukemia, or breast cancer.

Another aspect of the present disclosure provides for using the p300/CBP inhibitory compositions disclosed herein to treat, prevent, or ameliorate a symptom associated with a chronic inflammatory disorder or condition, including but not limited to asthma, inflammatory bowel disease (Crohn's disease or ulcerative colitis), chronic obstructive pulmonary disease, rheumatoid arthritis, and psoriasis.

Another aspect of the present disclosure provides for methods of treating, preventing, or ameliorating a symptom associated with a viral infection, including, but not limited to human immunodeficiency virus, hepatitis C virus, and human papilloma virus.

Yet another aspect of the present disclosure provides for methods of treating, preventing, or ameliorating a symptom associated with metabolic disease, including but not limited to: obesity, hepatic steatosis, dyslipidemia, hypertension, coronary heart disease, hepatic inflammation, and diabetes mellitus type 2.

Another aspect of the present disclosure provides for methods of treating, preventing, or ameliorating a symptom associated with a fibrotic disease or disorder. Fibrotic diseases and disorders include, for example, radiation-induced pneumonitis, radiation fibrosis, acute respiratory distress syndrome, chronic obstructive pulmonary disease, idiopathic pulmonary fibrosis, interstitial lung disease, myocardial infarction, ischemic stroke, ischemic kidney disease, transplant rejection, Leishmaniasis, type I diabetes, rheumatoid arthritis, chronic hepatitis, cirrhosis, inflammatory bowel disease, Crohn's disease, scleroderma, keloid, post-operative fibrosis, chemotherapy induced fibrosis (e.g., chemotherapy induced pulmonary fibrosis or ovarian cortical fibrosis), nephrogenic systemic fibrosis, retroperitoneal fibrosis, myelofibrosis, mediastinal fibrosis, cystic fibrosis, asbestosis, asthma, and pulmonary hypertension.

Another aspect of the present disclosure provides for methods of treating, preventing, or ameliorating a symptom associated with cardiac disease, including but not limited to cardiac hypertrophy and heart failure.

The present disclosure also relates to methods of treating, preventing, or ameliorating a symptom associated with a disease, disorder, or pathological condition involving p300/CBP comprising administering the compounds described herein in a therapeutically effective amount to a subject in need thereof as part of a combination therapy. It is apparent to a person of skill in the medical arts that agent(s) administered with the p300/CBP compounds disclosed herein are selected based upon the subject's disease, disorder, or pathological condition. For example, a subject with cancer may be administered a p300/CBP compound disclosed herein and an anti-cancer agent. In another example, a subject with a chronic inflammatory disease may be administered a p300/CBP compound disclosed herein and an anti-inflammatory agent.

In certain embodiments, the p300/CBP compound disclosed herein is administered to a subject with cancer in conjunction with other conventional cancer therapies such as radiation treatment or surgery. Radiation therapy is well-known in the art and includes X-ray therapies, such as gamma-irradiation, and radiopharmaceutical therapies.

In certain embodiments, a p300/CBP inhibitor is used with at least one anti-cancer agent. Anti-cancer agents include chemotherapeutic drugs. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor.

Chemotherapeutic agents include, for example, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonat-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors ofthrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

In certain embodiments, a p300/CBP inhibitor is used simultaneously, in the same formulation or in separate formulations, or sequentially with an additional agent(s) as part of a combination therapy regimen.

Accordingly, various embodiments of the present disclosure are directed to a method for treating a HAT dependent condition in a mammal in need thereof, the method comprising administering an effective amount of the pharmaceutical composition described herein (e.g., as described in the foregoing section entitled “Pharmaceutical Compositions and Administration”). In some embodiments, the condition is cancer, metabolic disease, neurogenerative disorders or inflammation. In some more specific embodiments, the condition is cancer (e.g., the various cancers described herein above). In other more specific embodiments, the compound is a compound as described in the foregoing section entitled “Compounds,” including the exemplary compounds provided in Table 1.

### Assays for Detecting HAT Activity

Methods for detecting HAT activity are well known in the art, and a variety of HAT assay kits are commercially available. For example, filter-binding assays measure the transfer of radiolabeled acetate from acetyl-CoA to protein, and continuous, spectroscopic enzyme coupled assays link the HAT reaction to the reduction of NAD+ by pyruvate or α-ketoglutarate dehydrogenase (Berndsen and Denu, 2005, Methods 36:321-333).

Scintillation proximity assays (SPA) using low energy radioisotopes permit rapid and sensitive analysis of a wide range of biological processes and is well suited for high-throughput screening of HAT inhibitors (see, e.g., Aherne et al., 2002, Methods 26:245-253; Turlais et al., 2001, Anal. Biochem. 298:62-68). In brief, beads or plates coated with capture molecule (target) is incubated with radiolabeled ligand. When the radiolabeled ligand is attached or in proximity to bead or plate surface, light emission is stimulated and measured by a photometer.

In a particular embodiment, compounds disclosed herein may be screened by using a radiolabel that is incorporated into a biotinylated form of the substrate as a result of an enzymatic reaction (i.e., acetylation). The reaction contents are then incubated on a specially manufactured multi-well plate (Perkin-Elmer), where the wells have been precoated with avidin and a scintillant. Alternatively, beads precoated with avidin and scintillant may be used instead of plates. The tight interaction of biotin-avidin complexes brings the radiolabel on the reaction product in close proximity to the scintillant, resulting in emission of a light signal. The need for the proximity of the radiolabel to the scintillant to generate a signal enables a rapid readout without elaborate post-assay work up. The interference from unreacted (hence free) radiolabel is minimal.

Particular methods for testing the activity of the compounds of the invention are described in more detail in the examples.

The following examples are provided for purpose of illustration and not limitation.

### Prep-SFC Separation (for Chiral Separation)

SFC purification was carried out using a modified Berger Instruments PrepSFC™ system. A manual version of the Berger system was integrated with a Gilson 232 autosampler for sample injection and a Cavro MiniPrep™ pipettor customized for fraction collection at atmospheric pressure (Olson, J.; Pan, J.; Hochlowski, J.; Searle, P.; Blanchard, D. JALA 2002, 7, 69-74). Custom designed collection shoes allowed collection into 18×150 mm tubes and a methanol wash system allows washing of shoes between fractions to maximize recovery and avoid cross-contamination of fractions. The system was controlled using SFC ProNTo™ software (version 1.5.305.15) and an Abbvie developed Visual Basic application for autosampler and fraction collector control. The outlet pressure was 100 bar, oven temperature at 35° C., and mobile phase flow rate at 40 mL/minute. Samples were injected as solutions in 1.5 mL CH₃OH solution unless noted otherwise. The preparative SFC system was controlled using SFC ProNTo™ software (version 1.5.305.15) and custom software for autosampler and fraction collector control. Fractions were collected based upon UV signal threshold and on-line Thermo MSQ mass spectrometry was used for molecular mass confirmation, using ESI ionization in positive mode. Mass spectra were acquired using a Navigator4.0 software and an Abbvie developed Visual Basic interface to communicate with SFC controlling software.

Abbreviations which may have been used herein include: AcOH for acetic acid, Ac₂O for acetic anhydride, AIBN for 2,2′-azobis(2-methylpropionitrile), BH₃-DMS for borane dimethylsulfide complex, BF₃OEt₂ for boron trifluoride diethyl ether complex, Boc for tert-butoxycarbonyl, (Boc)₂O for di-tert-butyl dicarbonate, n-BuLi for n-butyllithium, DBU for 1,8-diazabicyclo[5.4.0]undec-7-ene, DBAD for di-tert-butyl azodicarboxylate; DIAD for diisopropyl azodicarboxylate; DCC is 1,3-dicyclohexylcarbodiimide, DDQ for 2,3-dichloro-5,6-dicyano-1,4-benzoquinone, DEPBT is 3-(diethoxyphosphoryloxy)-1,2,3-benzotriazin-4(3H)-one, DMA for N,N-dimethylacetamide, DMAP for 4-dimethylaminopyridine, DME for 1,2-dimethoxyethane, DPPA is diphenylphosphine azide, DIBAL for diisobutylaluminum hydride, DMAP for 4-(dimethylamino)pyridine, DME for dimethoxyethane, DMF for N,N-dimethylformamide, DMPU for 1,3-dimethyl-3,4,5,6-tetrahydro-2(1H)-pyrimidinone; DMSO for dimethylsulfoxide, DIPEA for diisopropylethylamine, DPPA for diphenylphosphoryl azide, EDAC or EDCI or EDC for 1-ethyl-3-[3-(dimethylamino)propyl]-carbodiimide hydrochloride, EtOAc for ethyl acetate, EtOH for ethanol, HATU for O-(7-azabenzotriazol-1-yl)-N,N,N′,N′-tetramethyluronium hexafluorophosphate, HOBT is 1-hydroxybenzotriazole hydrate, LAH for lithium aluminum hydride, LDA for lithium diisopropylamide, MeOH for methanol, NaHMDS for sodium bis(trimethylsilyl)amide, NMMO or NMO for N-methylmorpholine N-oxide, NMP for 1-methyl-2-pyrrolidinone, Pd(dppf)₂C₂ for [1,1′-bis(diphenylphosphino)ferrocene]dichloropalladium(II), i-Pr for isopropyl, pyr for pyridine, TBAF is tetrabutyl ammonium fluoride, TFA for trifluoroacetic acid, THF for tetrahydrofuran, THP for tetrahydropyran, TPAP for tetrapropylammonium perruthenate, p-TsOH for para-toluenesulfonic acid monohydrate, DBAD for di-tert-butyl azodicarboxylate, DEAD for diethyl azodicarboxylate, DIAD for diisopropyl azodicarboxylate, BOP for benzotriazol-1-yloxytris(dimethylamino)phosphonium hexafluorophosphate, pyBOP for benzotriazol-1-yloxytripyrrolidinophosphonium hexafluorophosphate, BINAP for 2,2′-bis(diphenylphosphino)-1,1′-binaphthyl, CDI for 1,1′-carbonyldiimidazole or N,N′-carbonyldiimidazole, CyMAP for 2-dicyclohexylphosphino-2′-dimethylamino-1,1′-biphenyl, (R)-MOP for (R)-(+)-2-(diphenylphosphino)-2′-methoxy-1,1,1-binaphthyl, MOM-Cl for chloromethyl methyl ether, dppf for 1,1′-bis(diphenylphosphino)ferrocene, dba for dibenzylideneacetone, Pd(PPh₃)₄ for tetrakis(triphenylphosphine)palladium(0), PdCl₂dppf Cl₂CH₂ for [1,1′-bis(diphenylphosphino)ferrocene]dichloropalladium(II) dichloromethane, PdCl₂(dppf) for [1,1′-bis(diphenylphosphino)ferrocene]dichloropalladium(II), Pd₂(dba)₃ for tris(dibenzylideneacetone)dipalladium(0), (Tol)₃P for Pd(OAc)₂ for palladium(II) acetate, PdCl₂(PPh₃)₂ for bis(triphenylphosphine)palladium(II) dichloride, TsCl for toluene-2-sulfonyl chloride, t-Bu₃P for tri-tert-butylphosphine, 1,1′-carbonyldiimidazole (CDI), MsCl for mesyl chloride, pyBop for benzotriazol-1-yl-oxytripyrrolidinophosphonium hexafluorophosphate, DIEA for N,N-diisopropylethylamine, OTf for CF₃SO₃—, NCS for N-chlorosuccinimide, LHMDS for lithium hexamethyldisilazide, h or hr or hrs for hour(s), min for minutes, DCM for dichloromethane, and mCPBA for 3-chloroperoxybenzoic acid

### Example 1

### Preparation of Synthetic Intermediates

### Intermediate 1

### 2′,3′-dihydrospiro[imidazolidine-4,1′-indene]-2,5-dione (Intermediate 1)

To a stirring solution of SM1 (90.0 g, 68.0 mmol) in water (90 mL) and ethanol(90 mL) was added (NH₄)₂CO₃ (65.3 g, 680 mmol) and NaCN (10.0 g, 204 mmol) at room temperature. The reaction mixture was heated at 70° C. overnight. After being cooled to room temperature and concentrated, the residue was purified by silica gel column chromatography eluting with 100% ethyl acetate/Hexane to afford compound Intermediate-1 (9.00 g, 65%). LC-MS: m/z=203.2 [M+H]⁺

### Intermediate 2

### ethyl 1-amino-2,3-dihydro-1H-indene-1-carboxylate (2)

A suspension of Intermediate 1 (5.70 g, 27.8 mmol) and Ba(OH)₂.8H₂O (13.5 g, 42.7 mmol) in distilled water (114 mL) was heated at 150° C. in a sealed steel reactor for 72 hours. The reaction mixture was cooled to room temperature and acidified to pH 1.6 with H₂SO₄ (1 M). The precipitate was filtered off and the filtrate was evaporated to dryness. The residue was suspended in ethanol (200 mL) and thionyl chloride (2.47 mL, 33.8 mmol) was added at 0° C. The reaction mixture was heated to reflux for 24 h before being evaporated to dryness. Water (80 mL) was then added and the pH was adjusted with aqueous NH₄OH (25%) to pH 9-10 (indicated by pH paper). The aqueous solution was extracted with CH₂Cl₂ (3×50 mL) and the combined organic layers were dried with anhydrous Na₂SO₄, filtered, and evaporated, affording Compd 2 (4.18 g, 20.4 mmol, 73%). LC-MS: m/z=206.0 [M+H]⁺

### (S)-butyl 1-(2,2,2-trifluoroacetamido)-2,3-dihydro-1H-indene-1-carboxylate (3)

CAL-B (4.5 g, 50 mg/mL) was added to a solution of Compd 2 (0.90 g, 4.39 mmol) and BuCO₂Bu (6.34 g, 44.00 mmol) in DIPE (90.0 mL) in the presence of 4 Å MS (30 mg/mL). The reaction was stopped at 49% conversion by filtering off the enzyme after 24 h. (CF3CO)₂O (5.26 g, 21.80 mmol) in CH₂Cl₂ (60 mL) was added to the above filtrate and the reaction mixture was stirred for 2 hours before evaporating the solvent. The residue was purified on a silica gel column (petroleum ether/ethyl acetate, 20:1), affording oily N-trifluoroacetylated Compd-3 (Rf=0.17; 0.69 g, 0.70 mmol, 48%, ee 99%). LC-MS: m/z=330.2 [M+H]⁺

### (S)-1-amino-2,3-dihydro-1H-indene-1-carboxylic acid hydrochloride (4)

Hydrolysis of Compd-3 (81.0 mg, 0.17 mmol) in aqueous HCl (6 M) for 2 days in reflux produced Compd-4 (43.0 mg, 0.24 mmol, 99%). LC-MS: m/z=178.0 [M+H]⁺

To a stirring solution of compound 4 (0.2 g, 1.13 mmol) in MeOH (10 mL) was added SOCl₂ (266 mg, 2.26 mmol) at 0° C. and the reaction mixture was stirred at room temperature for 30 min. The solvent was removed under reduced pressure to afford the desired product Intermediate-2 (0.20 g, 93%) as an oil. TLC: 50% EtOAc/hexane (Rf: 0.2)

### Intermediate 3

### 5′-bromo-2′,3′-dihydrospiro[imidazolidine-4,1′-indene]-2,5-dione (2)

To a stirring solution of compound 1 (11.7 g, 55.4 mmol) in 60% EtOH/H₂O (80 mL) was added (NH₄)₂CO₃ (15.9 g, 0.17 mol) followed by potassium cyanide (5.4 g, 83.5 mmol) at room temperature. The reaction mixture was heated to 70° C. for 16 h. After consumption of the starting material (by TLC), the reaction mixture was diluted with water and extracted with EtOAc. The combined organic layers was dried over anhydrous Na₂SO₄ and concentrated under reduced pressure to obtain crude product, which was purified by silica gel column chromatography eluting with 5% MeOH/DCM to afford compound 2 (12.7 g, 81%). LC-MS: m/z=281.1/283.1 [M+H]⁺

### 1-amino-5-bromo-2,3-dihydro-1H-indene-1-carboxylic acid (3)

A mixture of compound 2 (11.7 g, 41.7 mol), Ba(OH)₂.8H₂O (40.2 g, 0.1 mol) in water (90 mL) was heated to 160° C. in a sealed tube for 3 days. The mixture was cooled to room temperature, and adjust PH to 1.5 by adding 1 M H₂SO₄, filtered and the water was concentrated under reduce pressure to obtain crude product 3, which was used to the next step without any other purification. (10.4 g, 90%). LC-MS: m/z=256.1/258.1 [M+H]⁺

### ethyl 1-amino-5-bromo-2,3-dihydro-1H-indene-1-carboxylate (4)

To a stirring solution of compound 3 (10.4 g, 40.2 mmol) in EtOH (140 mL) was added thionyl chloride (20 mL) at 0° C. and stirred at room temperature overnight. The reaction mixture was concentrated under reduced pressure to obtain crude product, which was used to the next step without any other purification as compound 4 (11.4 g, 73%) as an off-white solid. LC-MS: m/z=284.1/286.1 [M+H]⁺

### (S)-butyl 5-bromo-1-(2,2,2-trifluoroacetamido)-2,3-dihydro-1H-indene-1-carboxylate (5)

CAL-B preparation (50 mg/mL) was added to a solution of compound 4 (11.4 g, 40.1 mmol) and PrCO₂Bu (40 ml) in the presence of 4 Å MS (30 mg/mL). The reaction was stopped at 49% conversion by filtering off the enzyme after 2 days. (CF₃CO)₂O (20 mL) in DCM (100 mL) was added to the above filtrate and the reaction mixture was stirred for 2 hours before evaporation of the solvent. The residue was purified on a silica gel column (PE/EA, 20:1), affording oily compound 5 (6.4 g, 45%). LC-MS: m/z=408.1/410.1 [M+H]⁺

### (S)-1-amino-5-bromo-2,3-dihydro-1H-indene-1-carboxylic acid (6)

To a stirring solution of compound 5 (6.4 g, 15.7 mmol) in EtOH (60 mL) was added cont. HCl (60 mL). The reaction mixture was refluxing overnight. After consumption of the starting material (by TLC), the reaction mixture was concentrated under reduced pressure to obtain crude compound 6 (5.1 g, 83%) as a yellow oil, which was used in the next step without any other purification. LC-MS: m/z=256.1/258.1 [M+H]⁺

### (S)-methyl 1-amino-5-bromo-2,3-dihydro-1H-indene-1-carboxylate (Intermediate 3)

To a stirring solution of compound 6 (5.1 g, 20 mmol) in MeOH (60 mL) was added thionyl chloride (10 mL) at 0° C. and stirred at room temperature overnight. The reaction mixture was concentrated under reduced pressure to obtain crude product, which was used to the next step without any other purification as intermediate 3 (5.4 g, 85%) as brown oil.

### Intermediate 4

### (S)—N-benzyl-1-cyclopropylethanamine (2)

To a stirring solution of (S)-1-cyclopropylethanamine 1 (3.0 g, 35.2 mmol) in MeOH (30 mL) was added benzaldehyde (2.0 g, 18.8 mmol) followed by NaBH(OAc)₃ (4.0 g, 18.5 mmol) at room temperature. The reaction mixture was stirred at room temperature overnight. After consumption of the starting material (by TLC), the reaction mixture was diluted with 1N NaOH (50 mL) and extracted with DCM. Combined organic extracts were dried over anhydrous Na₂SO₄ and concentrated under reduced pressure to obtain crude product, which was purified by silica gel column chromatography eluting with 5% MeOH/DCM to afford compound 2 (5.1 g, 90%). LC-MS: m/z=176.2 [M+H]⁺

### (S)—N-benzyl-2-bromo-N-(1-cyclopropylethyl)acetamide (3)

To a stirring solution of (S)—N-benzyl-1-cyclopropylethanamine 2 (70 mg, 0.4 mmol) in DCM (30 mL) cooled to 0° C. was added 2-bromoacetyl bromide (0.1 g, 0.48 mmol), the mixture was warmed to room temperature and stirred for 1 hour. After consumption of the starting material (by TLC), the solvent from reaction mixture was quenched by saturated NaHCO₃ solution and extracted with DCM. Combined organic extracts were dried over anhydrous Na₂SO₄ and concentrated under reduced pressure to obtain crude product 3 (0.13 g, 88%), which was used to the next step without any other purification. LC-MS: m/z=296.2/298.2 [M+H]⁺

### (S)-2-azido-N-benzyl-N-(1-cyclopropylethyl)acetamide (4)

To a stirring solution of (S)—N-benzyl-2-bromo-N-(1-cyclopropylethyl)acetamide 3 (70 mg, 0.25 mmol) in DMSO (20 mL) was added NaN₃ (34 mg, 0.3 mmol) and stirred at room temperature for 1 hour. The reaction mixture was diluted with cold water and extracted with EtOAc. Combined organic extracts were dried over anhydrous Na₂SO₄ and concentrated under reduced pressure to obtain crude product 4 (0.1 g, 93%), which was used to the next step without any other purification. LC-MS: m/z=259.1 [M+H]⁺

### (S)-2-amino-N-benzyl-N-(1-cyclopropylethyl)acetamide (Intermediate-4)

To a stirring solution of (S)-2-azido-N-benzyl-N-(1-cyclopropylethyl)acetamide 4 (0.1 g, 0.25 mmol) in 80% THF/H₂O (5 mL) was added PPh₃ (0.12 g, 0.3 mmol) at 40° C. and stirred for 1 hour. After consumption of the starting material (by TLC), the reaction mixture was diluted with cold water and extracted with DCM. Combined organic extracts were dried over anhydrous Na₂SO₄ and concentrated under reduced pressure to obtain crude product, which was purified by silica gel column chromatography to afford Intermediate-4 (70 mg, 87%) as a yellow oil. LC-MS: m/z=233.2 [M+H]⁺

### Intermediate 5

### (R)—N-benzyl-1-cyclopropylethanamine (2)

To a stirring solution of (R)-1-cyclopropylethanamine 1 (0.8 g, 9.4 mmol) in MeOH (30 mL) was added benzaldehyde (2.0 g, 18.8 mmol) followed by NaBH(OAc)₃ (4.0 g, 18.5 mmol) at room temperature. The reaction mixture was stirred at room temperature overnight. After consumption of the starting material (by TLC), the reaction mixture was diluted with 1N NaOH (50 mL) and extracted with DCM. Combined organic extracts were dried over anhydrous Na₂SO₄ and concentrated under reduced pressure to obtain crude product, which was purified by silica gel column chromatography eluting with 5% MeOH/DCM to afford compound 2 (1.0 g, 77%). LC-MS: m/z=176.2 [M+H]⁺

### (R)—N-benzyl-2-bromo-N-(1-cyclopropylethyl)acetamide (3)

To a stirring solution of (R)—N-benzyl-1-cyclopropylethanamine 2 (2.9 g, 18 mmol) in DCM (30 mL) cooled to 0° C. was added 2-bromoacetyl bromide (4.0 g, 19.8 mmol), the mixture was warmed to room temperature and stirred for 1 hour. After consumption of the starting material (by TLC), the solvent from reaction mixture was quenched by saturated NaHCO₃ solution and extracted with DCM. Combined organic extracts were dried over anhydrous Na₂SO₄ and concentrated under reduced pressure to obtain crude product 3 (4.3 g, 88%), which was used to the next step without any other purification. LC-MS: m/z=296.2/298.2 [M+H]⁺

### (R)-2-azido-N-benzyl-N-(1-cyclopropylethyl)acetamide (4)

To a stirring solution of (R)—N-benzyl-2-bromo-N-(1-cyclopropylethyl)acetamide 3 (4.3 g, 16.2 mmol) in DMSO (20 mL) was added NaN₃ (1.22 g, 19.6 mmol) and stirred at room temperature for 1 hour. The reaction mixture was diluted with cold water and extracted with EtOAc. Combined organic extracts were dried over anhydrous Na₂SO₄ and concentrated under reduced pressure to obtain crude product 4 (4.0 g, 93%), which was used to the next step without any other purification. LC-MS: m/z=259.1 [M+H]⁺

### (R)-2-amino-N-benzyl-N-(1-cyclopropylethyl)acetamide (Intermediate 5)

To a stirring solution of (R)-2-azido-N-benzyl-N-(1-cyclopropylethyl)acetamide 4 (4.0 g, 15.2 mmol) in 80 THF/H₂O (25 mL) was added PPh₃ (4.5 g, 17 mmol) at 40° C. and stirred for 1 hour. After consumption of the starting material (by TLC), the reaction mixture was diluted with cold water and extracted with DCM. Combined organic extracts were dried over anhydrous Na₂SO₄ and concentrated under reduced pressure to obtain crude product, which was purified by silica gel column chromatography to afford Intermediate-5 (2.3 g, 67%) as an yellow oil. LC-MS: m/z=233.2 [M+H]⁺

### Intermediate 6

### (S)-1-cyclobutylethanamine (2)

To a stirring solution of(S)—N-1-cyclobutylethyl)-2-methylpropane-2-sulfinamide 1 (0.1 g, 0.45 mmol) in MeOH (20 mL) was added 4N HCl in dioxane (10 mL) and stirred at room temperature for 3 hours. After consumption of the starting material (by TLC), the reaction mixture was concentrated under reduced pressure to obtain crude product, which was washed with ether (3×10 mL) to afford compound 2 (60 mg, 87%) as off-white solid.

### (S)—N-benzyl-1-cyclobutylethanamine (3)

To a stirring solution of (S)-1-cyclobutylethanamine 2 (74 mg, 0.55 mmol) in MeOH (30 mL) was added benzaldehyde (0.11 g, 1.1 mmol) followed by NaBH(OAc)₃ (0.23 g, 1.1 mmol) at room temperature. The reaction mixture was stirred at room temperature overnight. After consumption of the starting material (by TLC), the reaction mixture was diluted with 1N NaOH (10 mL) and extracted with DCM. Combined organic extracts were dried over anhydrous Na₂SO₄ and concentrated under reduced pressure to obtain crude product, which was purified by silica gel column chromatography eluting with 5% MeOH/DCM to afford compound 3 (0.1 g, 70%). LC-MS: m/z=190.2 [M+H]⁺

### (S)—N-benzyl-2-bromo-N-(1-cyclobutylethyl)acetamide (4)

To a stirring solution of (S)—N-benzyl-1-cyclobutylethanamine 3 (140 mg, 0.7 mmol) in DCM (10 mL) cooled to 0° C. was added 2-bromoacetyl bromide (0.16 g, 0.8 mmol), the mixture was warmed to room temperature and stirred for 1 hour. After consumption of the starting material (by TLC), the solvent from reaction mixture was quenched by saturated NaHCO₃ solution and extracted with DCM. Combined organic extracts were dried over anhydrous Na₂SO₄ and concentrated under reduced pressure to obtain crude product 4 (0.17 g, 71%), which was used to the next step without any other purification. LC-MS: m/z=310.2/312.2 [M+H]⁺

### (S)-2-azido-N-benzyl-N-(1-cyclobutylethyl)acetamide (5)

To a stirring solution of (S)—N-benzyl-2-bromo-N-(1-cyclobutylethyl)acetamide 4 (220 mg, 0.71 mmol) in DMSO (20 mL) was added NaN₃ (58 mg, 0.9 mmol) and stirred at room temperature for 1 hour. The reaction mixture was diluted with cold water and extracted with EtOAc. Combined organic extracts were dried over anhydrous Na₂SO₄ and concentrated under reduced pressure to obtain crude product 5 (0.13 g, 83%), which was used to the next step without any other purification. LC-MS: m/z=273.1 [M+H]⁺

### (S)-2-amino-N-benzyl-N-(1-cyclobutylethyl)acetamide (Intermediate 6)

To a stirring solution of (S)-2-azido-N-benzyl-N-(1-cyclobutylethyl)acetamide 5 (0.15 g, 0.6 mmol) in 80% THF/H₂O (5 mL) was added PPh₃ (0.29 g, 1.1 mmol) at 40° C. and stirred for 1 hour. After consumption of the starting material (by TLC), the reaction mixture was diluted with cold water and extracted with DCM. Combined organic extracts were dried over anhydrous Na₂SO₄ and concentrated under reduced pressure to obtain crude product, which was purified by silica gel column chromatography to afford Intermediate-6 (100 mg, 67%) as a yellow oil. LC-MS: m/z=247.2 [M+H]⁺

### Intermediate 7

To a stirring solution of N—((R)-1-cyclobutylethyl)-2-methylpropane-2-sulfinamide 1 (0.15 g, 0.74 mmol) in MeOH (20 mL) was added 4N HCl in dioxane (10 mL) and stirred at room temperature for 3 hours. After consumption of the starting material (by TLC), the reaction mixture was concentrated under reduced pressure to obtain crude product, which was washed with ether (3×10 mL) to afford compound 2 (65 mg, 72%) as off-white solid.

### (R)—N-benzyl-1-cyclobutylethanamine (3)

To a stirring solution of (R)-1-cyclobutylethanamine 2 (75 mg, 0.55 mmol) in MeOH (30 mL) was added benzaldehyde (0.11 g, 1.1 mmol) followed by NaBH(OAc)₃ (0.23 g, 1.1 mmol) at room temperature. The reaction mixture was stirred at room temperature overnight. After consumption of the starting material (by TLC), the reaction mixture was diluted with 1N NaOH (10 mL) and extracted with DCM. Combined organic extracts were dried over anhydrous Na₂SO₄ and concentrated under reduced pressure to obtain crude product, which was purified by silica gel column chromatography eluting with 5% MeOH/DCM to afford compound 3 (0.1 g, 70%). LC-MS: m/z=190.2 [M+H]⁺

### (R)—N-benzyl-2-bromo-N-(1-cyclobutylethyl)acetamide (4)

To a stirring solution of (R)—N-benzyl-1-cyclobutylethanamine 3 (100 mg, 0.6 mmol) in DCM (10 mL) cooled to 0° C. was added 2-bromoacetyl bromide (0.13 g, 0.64 mmol), the mixture was warmed to room temperature and stirred for 1 hour. After consumption of the starting material (by TLC), the solvent from reaction mixture was quenched by saturated NaHCO₃ solution and extracted with DCM. Combined organic extracts were dried over anhydrous Na₂SO₄ and concentrated under reduced pressure to obtain crude product 4 (0.17 g, 81%), which was used to the next step without any other purification. LC-MS: m/z=310.2/312.2 [M+H]⁺

### (R)-2-azido-N-benzyl-N-(1-cyclobutylethyl)acetamide (5)

To a stirring solution of (R)—N-benzyl-2-bromo-N-(1-cyclobutylethyl)acetamide 4 (170 mg, 0.58 mmol) in DMSO (20 mL) was added NaN₃ (47 mg, 0.7 mmol) and stirred at room temperature for 1 hour. The reaction mixture was diluted with cold water and extracted with EtOAc. Combined organic extracts were dried over anhydrous Na₂SO₄ and concentrated under reduced pressure to obtain crude product 5 (0.11 g, 83%), which was used to the next step without any other purification. LC-MS: m/z=273.1 [M+H]⁺

### (R)-2-amino-N-benzyl-N-(1-cyclobutylethyl)acetamide (Intermediate 7)

To a stirring solution of (R)-2-azido-N-benzyl-N-(1-cyclobutylethyl)acetamide 5 (0.11 g, 0.6 mmol) in 80% THF/H₂O (5 mL) was added PPh₃ (0.18 g, 0.7 mmol) at 40° C. and stirred for 1 hour. After consumption of the starting material (by TLC), the reaction mixture was diluted with cold water and extracted with DCM. Combined organic extracts were dried over anhydrous Na₂SO₄ and concentrated under reduced pressure to obtain crude product, which was purified by silica gel column chromatography to afford Intermediate-7 (65 mg, 47%) as an yellow oil. LC-MS: m/z=247.2 [M+H]⁺

### Intermediate 8

### N-(1-cyano-1-(trimethylsilyloxy)-2,3-dihydro-1H-inden-5-yl)acetamide (2)

To a stirring solution of N-(1-oxo-2,3-dihydro-1H-inden-5-yl)acetamide (Intermediate 38) (1.125 g, 6 mmol) in toluene and MeCN (25 mL/5 mL) was added ZnI₂ (200 mg, 0.6 mmol) and TMSCN (0.9 g, 9 mmol), the mixture was heated to 60° C. for 3 hours. After consumption of the starting material (by TLC), the solvent from reaction mixture was removed under reduced pressure. Obtained residue was diluted with NaHCO₃ aqueous and extracted with EtOAc. Combined organic extracts were dried over anhydrous Na₂SO₄ and concentrated under reduced pressure to obtain crude product, which was purified by silica gel column chromatography to afford compound 2 (1.2 g, 70%) as a yellow liquid. TLC: 50% PE/EA

### ethyl 5-acetamido-1-hydroxy-2,3-dihydro-1H-indene-1-carbimidate (3)

Compound 2 (1 g, 3.5 mmol) was dissolved with EtOH (30 mL), the mixture was cold to 0 degree and the excess of HCl gas was inlet, the mixture was stirred for 2 hours. After consumption of the SM2 (by LC-MS), the solvent from reaction mixture was removed under reduced pressure. Obtained residue was used directly for subsequent step.

### N-(2′,4′-dioxo-2,3-dihydrospiro[indene-1,5′-ozazolidine]-5-yl)acetamide (Intermediate 8)

To a stirring solution of compound 3 (the crude) in DCM (20 mL) was added TEA (0.4 g, 4 mmol) and triphosgene (0.24 g, 0.8 mmol) dissolved with DCM (10 mL), the mixture was stirred for 3 hours. After consumption of the starting material (by LC-MS), the mixture was controlled to pH=5 and stirred for 1 hour. The solvent from reaction mixture was removed under reduced pressure. Obtained residue was purified by silica gel column chromatography to afford intermediate 8 (0.28 g, 31%) as a yellow solid.

TLC: 10% MeOH/DCM. LC-MS: m/z 261 [M+H]⁺

### Intermediate 9

### (S)-methyl 1-isocyanato-2,3-dihydro-1H-indene-1-carboxylate (2)

To a solution of intermediate 2 (4.7 g, 24.6 mmol) in DCM (100 mL) was added TEA (2.98 g, 19.5 mmol) and triphosgene (9.85 g, 9.85 mmol) at 0° C. under N₂, the reaction mixture was stirred at room temperature for 2 hours. After consumption of the starting material (by TLC), the solution was use in next step without purification. TLC: 20% EtOAc/hexane (R_{f}: 0.7)

### (S)-methyl 1-(3-(2-tert-butoxy-2-oxoethyl)ureido)-2,3-dihydro-1H-indene-1-carboxylate (3)

To a solution of (S)-methyl 1-isocyanato-2,3-dihydro-1H-indene-1-carboxylate 2 (100 mL) was added tert-butyl 2-aminoacetate (3.57 g, 27.1 mmol) and stirred at room temperature for 2 hours. After consumption of the starting material (by TLC), concentrated under reduced pressure to obtain crude product, then EtOAc was added, the solid was filtered off and concentrated under reduced pressure to obtain crude product 3 (9.5 g, 110%) as white solid. TLC: 20% EtOAc/hexane (R_{f}: 0.2). LC-MS: m/z 349.0 [M+H]⁺ (90% purity)

### (S)-tert-butyl 2-(2,5-dioxo-2′,3′-dihydrospiro[imidazolidine-4,1′-indene]-1-yl)acetate (4)

To a solution of (S)-methyl 1-(3-(2-tert-butoxy-2-oxoethyl)ureido)-2,3-dihydro-1H-indene-1-carboxylate 3 (9.5 g, 24.6 mmol) in THF (100 mL) was added LiOH (24.6 mL, 1N in H₂O) at 0° C. and stirred at room temperature for 3 hours. After consumption of the starting material (by TLC), the reaction mixture was diluted with water and extracted with EtOAc. Combined organic extracts were dried over anhydrous Na₂SO₄ and concentrated under reduced pressure to obtain crude product 4 (6.35 g, 82%) as white solid. TLC: 35% EtOAc/hexane (R_{f}: 0.5). LC-MS: m/z=261.0.0 [M+H-t-Bu]⁺ (90% purity)

### (S)-2-(2,5-dioxo-2′,3′-dihydrospiro[imidazolidine-4,1′-indene]-1-yl)acetic acid (Intermediate 9)

To a solution of (S)-tert-butyl 2-(2,5-dioxo-2′,3′-dihydrospiro[imidazolidine-4,1′-indene]-1-yl)acetate 4 (6.35 g, 20 mmol) in DCM (50 mL) was added TFA (50 mL) and stirred at room temperature for 2 hours, the reaction mixture was concentrated under reduced pressure to obtain crude product (intermediate 9) (5.35 g, 102%) as white solid.

### Intermediate 10

### 5′-bromo-2′,3′-dihydrospiro[imidazolidine-4,1′-indene]-2,5-dione

To a stirring solution of compound 1 (11.7 g, 55.4 mmol) in 60% EtOH/H₂O (80 mL) was added (NH₄)₂CO₃ (15.9 g, 0.17 mol) followed by potassium cyanide (5.4 g, 83.5 mmol) at room temperature. The reaction mixture was heated to 70° C. for 16 h. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford intermediate 10 (12.7 g, 81%). LC-MS: m/z 282.1 [M+H]⁺

### Intermediate 11

### N-benzyl-1-cyclobutylethanamine (Intermediate-11)

To a stirring solution of phenylmethanamine SM1 (3 g, 27.6 mmol) and 1-cyclobutylethanone (2.7 g, 27.6) in dry THF (30 mL) was added tetraisopropoxytitanium (9.4 g, 33.1 mmol) at room temperature. The reaction mixture was stirred at room temperature for 2 hours then NaBH₄ (2.1 g, 55.2 mmol) was added. The reaction mixture was stirred at room temperature overnight then quenched with water (50 ml). The mixture was filtrated and washed with EtOAc, the filtrate was concentrated and purified by silica gel column chromatography eluting with 25% EtOAc/Hexane to afford Intermediate 11 (1.5 g, 30%). LC-MS: m/z 190.1 [M+H]⁺

### Intermediate 13

### N-benzyl-2-bromo-N-(1-cyclopropylethyl)acetamide

To a stirring solution of N-benzyl-1-cyclopropylethanamine (Intermediate 14) (10.0 g, 57.14 mmol) and 2-bromoacetyl bromide (11.6 g, 57.14 mmol) in DCM (150 mL) was added dropwise pyridine (7.63 g, 85.71 mmol) and stirred at room temperature for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 30% EtOAc/PE to afford Intermediate 13 (8.8 g, 52.4%) as an off-white solid.

### Intermediate 14

### N-benzyl-1-cyclopropylethanamine

To a solution of phenylmethanamine (10.7 g, 100 mmol) and 1-cyclopropylethanone 2 (8.4 g, 100 mmol) in MeOH (200 mL) was added titanium tetraisopropanolate (31.24 g, 110 mmol) and stirred at room temperature for 4 h. Then NaBH₄ (7.6 g, 200 mmol) was added and stirred at room temperature for 2 hours. Diluted with water and extracted with DCM. Combined organic extracts were dried over anhydrous MgSO₄ and concentrated under reduced pressure to obtain crude product, which was purified by silica gel column chromatography (EtOAc/Hexane 1:1) to afford intermediate 14 (11.7 g, 66%) as oil.

### Intermediate 15

### N-benzyl-2-bromo-N-(1-cyclobutylethyl)acetamide

To a stirring solution of compound Intermediate 11 (100 mg, 0.529 mmol) in DCM (1 mL) was added pyridine (63.8 mg, 0.794 mmol) followed by 2-bromoacetyl bromide (106.8 g, 0.529 mmol) at room temperature and stirred at room temperature for 20 min. The reaction mixture was diluted with water and extracted with DCM. Combined organic extracts were dried over anhydrous Na₂SO₄ and concentrated under reduced pressure to obtain crude product, which was purified by silica gel column chromatography eluting with 50% EtOAc/PE to afford Intermediate 15 (56 mg, 34%) as a clear oil.

### Intermediate 16

### N—((S)-1-cyclobutylethyl)-2-methylpropane-2-sulfinamide

### (E)-N-(cyclobutylmethylene)-2-methylpropane-2-sulfinamide (1)

To a stirring solution of SM1 (9.7 g, 115 mmol) in DCM (100 mL) was (S)-2-methylpropane-2-sulfinamide (11.1 g, 92 mmol) and PPTS (0.723 g, 2.88 mmol) followed by MgSO₄ (34.5 g, 288 mmol) at room temperature. The reaction mixture was stirred at room temperature overnight. After consumption of the starting material (by TLC), the reaction mixture was filtered and the filtrate was concentrated under reduced pressure to obtain crude product, which was purified by silica gel column chromatography eluting with 14% EtOAc/PE to afford compound 1 (14.32 g, 83%) as a clear oil. LC-MS: m/z=188 [M+H]⁺

To a stirring solution of compound 1 (14.32 g, 76.6 mmol) in DCM (150 mL) was added 3M methylmagnesium bromide in Et₂O (61.3 mL, 183.8 mmol) dropwise at −50° C. under nitrogen atmosphere. The reaction mixture was stirred at −48° C. for 3 hours and stirred at room temperature overnight. The resulting mixture was quenched by sat. NH₄Cl solution, extracted with EtOAc. Combined organic extracts were dried over anhydrous Na₂SO₄ and concentrated under reduced pressure to obtain crude product, which was purified by silica gel column chromatography eluting with 50% EtOAc/PE to afford Intermediate 16 (14.8 g, 95%) as a clear oil.

### Intermediate 17

### (E)-N-(cyclobutylmethylene)-2-methylpropane-2-sulfinamide (1)

To a stirring solution of SM1 (2 g, 23.8 mmol) in DCM (100 mL) was (R)-2-methylpropane-2-sulfinamide (1.44 g, 11.9 mmol) and PPTS (0.149 g, 0.595 mmol) followed by MgSO₄ (7.14 g, 59.5 mmol) at room temperature. The reaction mixture was stirred at room temperature overnight. After consumption of the starting material (by TLC), the reaction mixture was filtered and the filtrate was concentrated under reduced pressure to obtain crude product, which was purified by silica gel column chromatography eluting with 14% EtOAc/PE to afford compound 1 (2.27 g, 51%) as a clear oil. LC-MS: m/z=188 [M+H]⁺

### N—((R)-1-cyclobutylethyl)-2-methylpropane-2-sulfinamide

To a stirring solution of compound 1 (2.27 g, 12.1 mmol) in DCM (20 mL) was added 3M methylmagnesium bromide in Et₂O (9.68 mL, 29.04 mmol) dropwise at −50° C. under nitrogen atmosphere. The reaction mixture was stirred at −48° C. for 4 h and stirred at room temperature overnight. The resulting mixture was quenched by sat. NH₄Cl solution, extracted with EtOAc. Combined organic extracts were dried over anhydrous Na₂SO₄ and concentrated under reduced pressure to obtain crude product, which was purified by silica gel column chromatography eluting with 50% EtOAc/PE to afford Intermediate 17 (1.71 g, 70%) as a clear oil.

### Intermediate 18

### (S)-2-(5-acetamido-2′,4′-dioxo-2,3-dihydrospiro[indene-1,5′-ozazolidine]-3′-yl)-N-benzyl-N-(1-cyclopropylethyl)acetamide (3)

A solution of N-(2′,4′-dioxo-2,3-dihydrospiro[indene-1,5′-ozazolidine]-5-yl)acetamide (1 g, 3.85 mmol), (S)—N-benzyl-2-bromo-N-(1-cyclopropylethyl)acetamide (1.14 g, 3.85 mmol) and K₂CO₃ (1 g, 7.7 mmol) in DMF (20 mL) was stirred for 2 hours. After consumption of the starting material (by TLC), the reaction mixture was diluted with sat NaCl aqueous and extracted with EtOAc. Combined organic extracts were dried over anhydrous Na₂SO₄ and concentrated under reduced pressure to obtain crude product, which was purified by silica gel column chromatography to afford compound 3 (1.35 g, 73%) as a yellow liquid. TLC: 35% PE/EA

### 2-(5-amino-2′,4′-dioxo-2,3-dihydrospiro[indene-1,5′-ozazolidine]-3′-yl)-N-benzyl-N—((S)-1-cyclopropylethyl)acetamide (Intermediate 18)

Compound 3 (1.35 g, 2.85 mmol) was dissolved with MeOH/THF/H₂O (10 mL/10 mL/10 mL), and 37% HCl (4 mL) was added. The mixture was heated to 60 degrees C. and stirred for 2 hours. After consumption of compound 3 (by LC-MS), the solvent from reaction mixture was removed under reduced pressure the reaction mixture was diluted with sat NaCl aqueous and extracted with EtOAc. Combined organic extracts were dried over anhydrous Na₂SO₄ and concentrated under reduced pressure to obtain crude product, which was purified by silica gel column chromatography to afford intermediate 18 (1 g, 81%) as a yellow liquid. TLC: 35% PE/EA. ¹H NMR (400 MHz, DMSO-d₆): δ 7.65 (d, J=18.2 Hz, 1H), 7.51-7.08 (m, 7H), 6.56-6.41 (m, 2H), 4.75-4.20 (m, 5H), 4.13-3.54 (m, 1H), 3.52-2.74 (m, 2H), 2.74-2.33 (m, 1H), 1.42-1.00 (m, 3H), 1.02-0.84 (m, 1H), 0.56-0.16 (m, 4H). LC-MS: m/z=434.10 [M+H]⁺ (91.53% purity, 214 nm)

### Intermediate 19

### (R)-methyl 1-amino-2,3-dihydro-1H-indene-1-carboxylate ethyl 1-amino-2,3-dihydro-1H-indene-1-carboxylate (2)

A suspension of Intermediate 1 (5.70 g, 27.80 mmol) and Ba(OH)₂ 8H₂O (13.50 g, 42.71 mmol) in distilled water (114 mL) was heated at 150° C. in a sealed steel reactor for 72 hours. The reaction mixture was cooled to room temperature and acidified to pH 1.6 with H₂SO₄, (1 M). The precipitate was filtered off and the filtrate was evaporated to dryness. The residue was suspended in EtOH (200 mL) and SOCl₂ (2.47 mL, 33.82 mmol) was added at 0° C. The reaction mixture was heated to reflux for 24 h before being evaporated to dryness. Water (80 mL) was then added and the pH was adjusted with aqueous NH₄OH (25%) to pH 9-10 (indicated by pH paper). The aqueous solution was extracted with CH₂Cl₂ (3×50 mL) and the combined organic layers were dried with anhydrous Na₂SO₄, filtered, and evaporated, affording Compd 2 (4.18 g, 20.39 mmol, 73%). LC-MS: m/z=206.0 [M+H]⁺

### (R)-ethyl 1-(2,2,2-trifluoroacetamido)-2,3-dihydro-1H-indene-1-carboxylate (3)

CAL-B (4.5 g, 50 mg/mL) was added to a solution of Compd 2 (0.90 g, 4.39 mmol) and BuCO₂Bu (6.34 g, 44.00 mmol) in DIPE (90.0 mL) in the presence of 4 Å MS (30 mg/mL). The reaction was stopped at 49% conversion by filtering off the enzyme after 24 h. (CF₃CO)₂O (5.26 g, 21.80 mmol) in CH₂Cl₂ (60 mL) was added to the above filtrate and the reaction mixture was stirred for 2 hours before evaporating the solvent. The residue was purified on a silica gel column (petroleum ether/ethyl acetate, 20:1), affording oily Compd-3 (Rf=0.10; 0.62 g, 2.06 mmol, 47%, ee 92%). LC-MS: m/z=302.2 [M+H]⁺

### (R)-1-amino-2,3-dihydro-1H-indene-1-carboxylic acid hydrochloride (4)

Hydrolysis of Compd-3 (81.0 mg, 0.17 mmol) in aqueous HCl (6 M) for 2 days in reflux produced Compd-4 (42.0 mg, 0.24 mmol, 98%). LC-MS: m/z=178.0 [M+H]⁺

### (R)-methyl 1-amino-2,3-dihydro-1H-indene-1-carboxylate

To a stirring solution of compound 4 (0.2 g, 1.13 mmol) in MeOH (10 mL) was added SOCl₂ (266 mg, 2.26 mmol) at 0° C. and the reaction mixture was stirred at room temperature for 30 min. The solvent was removed under reduced pressure to afford the desired product Intermediate-19 (0.20 g, 93%) as an oil. TLC: 50% EtOAc/hexane (Rf: 0.2)

### Intermediate 20

### tert-butyl 2-(2,5-dioxo-2′,3′-dihydrospiro[imidazolidine-4,1′-indene]-1-yl)acetate (1)

To a stirring solution of compound Intermediate-1 (2.5 g, 12.4 mmol) in dry DMF (10 mL) was added K₂CO₃ (3.45 g, 25 mmol) and tert-butyl 2-bromoacetate (2.54 g, 13 mmol) at room temperature. The reaction mixture was heated at 90° C. for 1.5 hours. After being cooled to room temperature the reaction mixture was diluted with cold water and extracted with EtOAc. Combined organic extracts were dried over anhydrous Na₂SO₄ and concentrated under reduced pressure to obtain crude product, which was purified by silica gel column chromatography eluting with 33% EtOAc/Hexane to afford compound 1 (3 g, 76%). TLC: 100% EtOAc/Hexane (R_{f}: 0.7)

### 2-(2,5-dioxo-2′,3′-dihydrospiro[imidazolidine-4,1′-indene]-1-yl)acetic acid (Intermediate-20)

To a stirring solution of compound 1 (3 g, 9.5 mmol) in dry DCM (30 mL) was added TFA (10.8 g, 95 mmol) at room temperature. The reaction mixture was stirred at room temperature for 2 hours then concentrated under reduced pressure to afford compound Intermediate-20 (3.5 g, crude). LC-MS: m/z=261.2 [M+H]⁺

### Intermediate 21

### 3′-bromo-2′,3′-dihydrospiro[imidazolidine-4,1′-indene]-2,5-dione (1)

To a stirring solution of intermediate 1 (0.5 g, 2.5 mmol) in CCl₄ (10 mL) was added AIBN (41 mg, 0.25 mol) followed by NBS (0.53 g, 3.0 mmol) at room temperature. The reaction mixture was heated to 70° C. for 2 hours. After consumption of the starting material (by TLC), the reaction mixture was concentrated under reduced pressure to obtain crude product, which was purified by silica gel column chromatography eluting with 5% MeOH/DCM to afford compound 1 (0.7 g, 82%). LC-MS: m/z=281.1/283.1 [M+H]⁺

### spiro[imidazolidine-4,1′-indene]-2,5-dione

To a stirring solution of compound 1 (0.7 g, 2.2 mmol) in CCl₄ (10 mL) was added DBU (2 mL) at room temperature and stirred for 2 days. The reaction mixture was concentrated under reduce pressure to obtain crude product, which was purified by silica gel column chromatography eluting with 5% MeOH/DCM to afford intermediate 21 (0.2 g, 32%). LC-MS: m/z=201.1 [M+H]⁺

### Intermediate 22

### 2-bromo-N,N-dimethylacetamide (Intermediate-22)

To a stirring solution of dimethylamine hydrochloride (0.67 g, 10 mmol) and K₂CO₃ (2.8 g, 20 mmol) in dry DCM (30 mL) was added SM 2 (2 g, 10 mmol) at 0° C. The reaction mixture was stirred at room temperature overnight then filtrated and concentrated to afford Intermediate-22 (1 g, 26%). LC-MS: m/z=166.1/168.1 [M+H]⁺

### Intermediate 23 and 24

### (S)—N-benzyl-1-cyclopropylethanamine (Intermediate-24)

A mixture of (S)-1-cyclopropylethanamine SM1 (850 mg, 10.0 mmol) and benzaldehyde SM2 (1060 mg, 1.0 mmol) in methanol (8 mL) and 1,2-dichloroethane (8 mL) was stirred at room temperature for 1 hour. NaBH₃CN (2.6 g, 12 mmol) was added and the mixture was stirred at 40° C. for 1 hour. The reaction mixture was poured to NaHCO₃ (sat, aq, 10 mL) and then extracted with chloroform (20 mL×2). The organic phase was combined and concentrated under reduced pressure to obtain crude product, which was purified by column chromatography (dichloromethane-methanol=100:1) to afford (S)—N-benzyl-1-cyclopropylethanamine Intermediate-24 (1.3 g, 74%). ¹H NMR (300 MHz, CDCl₃): δ 7.46-7.17 (m, 5H), 3.84 (s, 2H), 1.98-1.81 (m, 1H), 1.64 (brs, 1H), 1.19 (d, J=6.3 Hz, 3H), 0.86-0.71 (m, 1H), 0.60-0.37 (m, 2H), 0.23-0.02 (m, 2H).

### (S)—N-benzyl-2-bromo-N-(1-cyclopropylethyl)acetamide (Intermediate-23)

To a stirring solution of (S)—N-benzyl-1-cyclopropylethanamine Intermediate-24 (200 mg, 1.1 mmol) in dichloromethane (6 mL) was added a solution of 2-bromoacetyl bromide (303 mg, 1.5 mmol) in dichloromethane (1 mL) at 0° C. by dropwise. The mixture was stirred at room temperature for 1 hour. The reaction mixture was poured to water (10 mL) and then extracted with chloroform (20 mL×2). The organic phase was combined and concentrated under reduced pressure to obtain crude product, which was purified by column chromatography (dichloromethane-methanol=100:1) to afford (S)—N-benzyl-2-bromo-N-(1-cyclopropylethyl)acetamide Intermediate-23 (225 mg, 70%) as oil. ¹H NMR (300 MHz, CDCl₃): δ 7.35-7.25 (m, 6H), 4.68 (s, 2H), 4.05-3.80 (m, 2H), 3.30-3.20 (m, 1H), 1.28-1.20 (m, 3H), 0.87-0.79 (m, 1H), 0.63-0.48 (m, 1H), 0.45-0.15 (m, 3H).

### Intermediate 25

### 5-bromo-1-(trimethylsilyloxy)-2,3-dihydro-1H-indene-1-carbonitrile (1)

To a stirring solution of 5-bromo-2,3-dihydro-1H-inden-1-one SM1 (50 g, 238 mmol) in toluene (500 mL), CH₃CN (250 ml), was added TMSCN (28.3 g, 286 mmol), followed by ZnI₂ (2.26 g, 7.14 mmol) at room temperature. The reaction mixture was heated to 75° C. for 16 h. After consumption of the starting material (by TLC), the reaction mixture was diluted with aq NaHCO₃ and extracted with EtOAc. Combined organic extracts were dried over anhydrous Na₂SO₄ and concentrated under reduced pressure to obtain crude product, which was purified by silica gel column chromatography eluting with 20% EA/PE to afford compound 1 (45 g, 61%). TLC: 20% EA/PE (R_{f}: 0.5)

### ethyl 5-bromo-1-hydroxy-2,3-dihydro-1H-indene-1-carbimidate hydrochloride (2)

A solution of 5-bromo-1-(trimethylsilyloxy)-2,3-dihydro-1H-indene-1-carbonitrile compound 1 (45 g, 145.6 mmol), in ethanol (500 mL) was cooled to 0° C. HCl (gas) was charged for 3 hours. After consumption of the starting material (by TLC), the solvent from reaction mixture was removed under reduced pressure. Obtained residue was washed with ether to afford compound 2 (33.5 g, 72%). LC-MS: m/z=284/286 [M+H]⁺

### 5-bromo-2,3-dihydrospiro[indene-1,5′-ozazolidine]-2′,4′-dione (Intermediate 25)

To a stirring solution of ethyl 5-bromo-1-hydroxy-2,3-dihydro-1H-indene-1-carbimidate hydrochloride compound 2 (20 g, 62.7 mmol) in dry THF (500 mL) was added Et₃N (19 g, 188.08 mmol) followed by phosgene (18.5 g, 62.7 mmol) at 0° C. and stirred at room temperature for 1 hour. The reaction mixture was diluted with 1N HCl, stirred at room temperature for 0.5 h, and extracted with EA. Combined organic extracts were dried over anhydrous Na₂SO₄ and concentrated under reduced pressure to obtain crude product, which was purified by silica gel column chromatography eluting with 30% EA/PE to afford Intermediate 25 (16 g, 90%). TLC: 30% EtOAc/Hexane (Rf: 0.4). LC-MS: m/z=282/284 [M+H]⁺

### Intermediate 26

### N-benzyl-2-(5′-bromo-2,5-dioxo-2′,3′-dihydrospiro[imidazolidine-4,1′-indene]-1-yl)-N-(1-cyclopropylethyl)acetamide

To a stirring solution of compound 1 (0.2 g, 0.59 mmol), Intermediate-14 (114 mg, 0.65 mmol) and DIPEA (114 mg, 0.89 mmol) in DMF (4 mL) was added HATU (0.27 mg, 0.71 mmol). The mixture was stirred at room temperature for 1 hour. The mixture was diluted with water and extracted with EtOAc. Combined organic extracts were dried over anhydrous Na2SO4 and concentrated under reduced pressure. The residue was purified by Prep-HPLC to afford compound Intermediate-26 (180 mg, 61%) as an off-white solid.

### Intermediate 27

### N-benzyl-2-(5′-bromo-2,5-dioxo-2′,3′-dihydrospiro[imidazolidine-4,1′-indene]-1-yl)-N—((S)-1-cyclopropylethyl)acetamide

To a stirring solution of Intermediate-10 (10.4 g, 37.2 mmol) and Intermediate-23 (10 g, 33.8 mmol) in DMF (50 mL) was added K₂CO₃ (9.33 g, 67.6 mmol) and stirred at room temperature for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 40% EtOAc/PE to afford compound Intermediate-27 (12 g, 72%) as an off-white solid.

### Intermediate 28

### (S)—N-benzyl-2-(5-bromo-2′,4′-dioxo-2,3-dihydrospiro[indene-1,5′-ozazolidine]-3′-yl)-N-(1-cyclopropylethyl)acetamide (Intermediate 28)

A solution of 5-bromo-2,3-dihydrospiro[indene-1,5′-ozazolidine]-2′,4′-dione SM1 (0.5 g, 1.77 mmol), (S)—N-benzyl-2-bromo-N-(1-cyclopropylethyl)acetamide SM2 (0.53 g, 1.77 mmol) and K₂CO₃ (0.49 g, 3.54 mmol) in DMF (20 mL) was stirred for 2 hours. After consumption of the starting material (by TLC), the reaction mixture was diluted with sat NaCl aqueous and extracted with EtOAc. Combined organic extracts were dried over anhydrous Na₂SO₄ and concentrated under reduced pressure to obtain crude product, which was purified by silica gel column chromatography to afford Intermediate 28 (0.82 g, 94%) as an off-white solid. TLC: 50% PE/EA. LC-MS: m/z=497/499 [M+H]⁺

### Intermediate 29

### (S)-1-cyclobutylethanamine hydrochloride

To a stirring solution of Intermediate 16 (500 mg, 2.46 mmol) in MeOH (2 mL) was added 2 M HCl/dioxane (3 mL). The reaction mixture was stirred at room temperature for 0.5 h. After consumption of the starting material (by TLC), the reaction mixture was concentrated under reduced pressure to obtain crude Intermediate 29 (300 mg, 90%) as a white solid.

### Intermediate 30

### 2-bromo-N-methylacetamide (Intermediate-30)

To a stirring solution of methanamine hydrochloride (3.4 g, 50 mmol) and K₂CO₃ (13.8 g, 100 mmol) in dry DCM (200 mL) was added SM 1 (10 g, 50 mmol) at 0° C. The reaction mixture was stirred at room temperature overnight then filtrated and concentrated and purified by silica gel column chromatography eluting with 10% MeOH/DCM to afford compound Intermediate-30 (2 g, 26%). TLC: 10% MeOH/DCM (R_{f}: 0.2). LC-MS: m/z=152.1/154.1 [M+H]⁺

### Intermediate 31

### 4′-bromo-2′,3′-dihydrospiro[imidazolidine-4,1′-indene]-2,5-dione (1)

To a stirring solution of 4-bromo-2,3-dihydro-1H-inden-1-one SM1 (1.3 g, 6.2 mmol) in water (20 mL) and MeOH (20 mL) was added (NH₄)₂CO₃ (3.0 g, 30.8 mmol) and KCN (2.0 g, 31 mmol) at room temperature. The reaction mixture was heated at 50° C. overnight. After being cooled to room temperature and concentrated, the residue was purified by silica gel column chromatography eluting with 50% EtOAc/Hexane to afford compound 1 (1 g, 57%). LC-MS: m/z=281.2/283.1 [M+H]⁺ should see two peaks 281 and 283

### tert-butyl 2-(4′-bromo-2,5-dioxo-2′,3′-dihydrospiro[imidazolidine-4,1′-indene]-1-yl)acetate (3)

To a stirring solution of compound 1 (1.0 g, 3.6 mmol) in dry DMF (10 mL) was added K₂CO₃ (0.99 g, 7.2 mmol) and tert-butyl 2-bromoacetate (0.77 g, 3.9 mmol) at room temperature. The reaction mixture was heated at 40° C. for 1.5 h. After being cooled to room temperature the reaction mixture was diluted with cold water and extracted with EtOAc. Combined organic extracts were dried over anhydrous Na₂SO₄ and concentrated under reduced pressure to obtain crude product, which was purified by silica gel column chromatography eluting with 33% EtOAc/Hexane to afford compound 3 (1.08 g, 76%).

TLC: 40% EtOAc/Hexane (R_{f}: 0.3)

### 2-(4′-bromo-2,5-dioxo-2′,3′-dihydrospiro[imidazolidine-4,1′-indene]-1-yl)acetic acid

To a stirring solution of compound 3 (0.7 g, 1.78 mmol) in dry DCM (5 mL) was added TFA (2.7 g, 23.7 mmol) at room temperature. The reaction mixture was stirred at room temperature for 2 hours then concentrated under reduced pressure to afford Intermediate 31 (0.45 g, crude).

### Intermediate 32

### (S)-methyl 5-bromo-1-isocyanato-2,3-dihydro-1H-indene-1-carboxylate (1)

To a stirring solution of intermediate 3 (1.4 g, 5.2 mmol) in DCM cooled to 0° C. and was added Et₃N (0.8 g, 7.8 mmol) followed by triphosgene (0.61 g, 2.1 mmol). The reaction mixture was stirred under N₂ for 1 hour. After consumption of the starting material (by TLC), the reaction mixture was used to the next step without any purification.

### (S)-methyl 1-(3-(2-(benzyl((S)-1-cyclopropylethyl)amino)-2-oxoethyl)ureido)-5-bromo-2,3-dihydro-1H-indene-1-carboxylate (2)

A mixture of compound 1 an equal volume of Et₃N and intermediate 4 (1.2 g, 5.2 mmol) was stirred under N₂ at room temperature overnight, After consumption of the starting material (by TLC), the solvent from reaction mixture was removed under reduced pressure. Obtained residue was used to the next step without any other purification. LC-MS: m/z=528.1/530.1 [M+H]⁺

### N-benzyl-2-((S)-5′-bromo-2,5-dioxo-2′,3′-dihydrospiro[imidazolidine-4,1′-indene]-1-yl)-N—((S)-1-cyclopropylethyl)acetamide

To a stirring solution of compound 2 (1.2 g, 2.7 mmol) in 30 mL THF was added 1N LiOH (8.1 mmol) and the mixture was stirred at room temperature for 3 hours, After consumption of the starting material (by TLC), the mixture was diluted with water and extracted with DCM, Combined organic extracts were dried over anhydrous Na₂SO₄ and concentrated under reduced pressure to obtain crude product, which was purified by silica gel column chromatography to afford intermediate 32 (1.1 g, 55%) as an off-white solid. LC-MS: m/z=496.1/498.1 [M+H]⁺

### Intermediate 33

### N-benzyl-N—((S)-1-cyclopropylethyl)-2-((S)-5′-(diphenylmethyleneamino)-2,5-dioxo-2′,3′-dihydrospiro[imidazolidine-4,1′-indene]-1-yl)acetamide (2)

To a suspension of intermediate 32 (0.1 g, 0.2 mmol) and diphenylmethanimine (0.072 g, 0.4 mmol) in toluene (20 mL) was added Pd₂(dba)₃ (18 mg, 0.02 mmol), BINAP (25 mg, 0.04 mmol) and t-BuONa (58 mg, 0.6 mmol) under N₂, the reaction mixture was stirred at 100° C. for 4 h. Cooled to room temperature and concentrated under reduced pressure to obtain crude product, which was purified by silica gel column chromatography (EtOAc/Hexane 1:2) to afford compound 2 (0.12 g, 100%) as white solid. ¹H NMR (300 MHz, CDCl₃) δ 7.77-7.67 (m, 2H), 7.53-7.35 (m, 5H), 7.33-7.22 (m, 5H), 7.11 (dd, J=7.4, 2.1 Hz, 3H), 6.75-6.52 (m, 3H), 4.74-4.61 (m, 2H), 4.38 (s, 1H), 4.23 (d, J=3.5 Hz, 1H), 4.04-3.94 (m, 1H), 2.87-2.66 (m, 2H), 2.31-2.16 (m, 2H), 1.21 (dd, J=14.7, 5.3 Hz, 3H), 0.86-0.75 (m, 1H), 0.58-0.47 (m, 1H), 0.41-0.20 (m, 3H). LC-MS: m/z=596.7 [M+H]⁺ (90% purity)

### 2-((S)-5′-amino-2,5-dioxo-2′,3′-dihydrospiro[imidazolidine-4,1′-indene]-1-yl)-N-benzyl-N—((S)-1-cyclopropylethyl)acetamide

To a solution of N-benzyl-N—((S)-1-cyclopropylethyl)-2-((S)-5′-(diphenylmethyleneamino)-2,5-dioxo-2′,3′-dihydrospiro[imidazolidine-4,1′-indene]-1-yl)acetamide 2 (120 mg, 0.2 mmol) in THF (15 mL) was added HCl (1N, 3 mL), then stirred at room temperature for 2 hours. After consumption of the starting material (by TLC) and concentrated under reduced pressure to obtain crude product, which was purified by silica gel column chromatography (DCM/MeOH 20:1) to afford intermediate 33 (65 mg, 75%) as brown solid.

### Intermediate 34

### (R)—N-benzyl-1-cyclopropylethanamine (Intermediate 34)

To a stirring solution of 1-cyclopropylethanone 1 (2.0 g, 16 mmol) in MeOH (30 mL) was added (4-fluorophenyl)methanamine (1.3 g, 16 mmol) followed by NaBH(OAc)₃ (1.0 g, 18.5 mmol) at room temperature. The reaction mixture was stirred at room temperature overnight. After consumption of the starting material (by TLC), the reaction mixture was diluted with 1N NaOH (50 mL) and extracted with DCM. Combined organic extracts were dried over anhydrous Na₂SO₄ and concentrated under reduced pressure to obtain crude product, which was purified by silica gel column chromatography eluting with 5% MeOH/DCM to afford intermediate 34 (1.2 g, 67%). LC-MS: m/z=194.2 [M+H]⁺

### Intermediate 35

### tert-butyl 2-(5′-bromo-2,5-dioxo-2′,3′-dihydrospiro[imidazolidine-4,1′-indene]-1-yl)acetate (Intermediate-35)

To a stirring solution of Intermediate-13 (3 g, 11.4 mmol) in DMF (20 mL) was added K₂CO₃ (3.14 g, 22.8 mmol) at room temperature. To this added compound SM1 (2.67 g, 13.7 mmol) at room temperature and the reaction mixture was stirred at room temperature for 1 hour. The mixture was diluted with brine, extracted with EtOAc, the organic phase was washed with brine, dried over anhydrous Na₂SO₄ and concentrated and purified by silica gel column chromatography eluting with 30% EtOAc/Hexane to afford compound Intermediate-35 (3.2 g, 71%) as off white solid. TLC: 50% EtOAc/Hexane (Rf: 0.5)

### Intermediate 36

### 2-amino-2-methylpropanoyl chloride hydrochloride (Intermediate-36)

To a stirring solution of SM2 (2.1 g, 24.4 mmol) in dry MeCN (40 ml) was added PCl₅ (6.9 g, 33 mmol) at room temperature. The reaction mixture was stirred at room temperature overnight the SM1 (2.1 g, 20 mmol) was added at room temperature and the resultant mixture was stirred at room temperature for another 22 h then filtrated and the precipitate was washed with MeCN and dried in vacuum to afford Intermediate-36 (2.8 g, 88%).

### Intermediate 37

### N-benzyl-N—((S)-1-cyclopropylethyl)-2-(5-(diphenylmethyleneamino)-2′,4′-dioxo-2,3-dihydrospiro[indene-1,5′-ozazolidine]-3′-yl)acetamide (1)

To a solution of diphenylmethanimine SM 1 (8.04 g, 44.4 mmol) and Intermediate-27 (11 g, 22.2 mmol) in toluene (150 mL) was added t-BuONa (6.4 g, 66.6 mmol), BINAP (2.74 g, 4.4 mmol) and Pd₂(dba)₃ (2.02 g, 2.2 mmol) under N₂. Then stirred at 90° C. for 4 h. After consumption of the starting material (by TLC), diluted with water and extracted with EtOAc. Combined organic extracts were dried over anhydrous MgSO₄ and concentrated under reduced pressure to obtain crude product, which was purified by silica gel column chromatography eluting with 40% EtOAc/PE to afford 1 (7 g, 53%) as white solid. LC-MS: m/z=597.3 [M+H]⁺

### 2-((S)-5-amino-2′,4′-dioxo-2,3-dihydrospiro[indene-1,5′-ozazolidine]-3′-yl)-N-benzyl-N-(1-cyclopropylethyl)acetamide

To a solution of 1 (7 g, 11.7 mmol) in THF (100 mL) was 1N HCl solution (35 ml) under N2. Then stirred at r.t for 2 hours. After consumption of the starting material (by TLC), diluted with water and extracted with EtOAc. Combined organic extracts were dried over anhydrous MgSO₄ and concentrated under reduced pressure to obtain crude product, which was purified by silica gel column chromatography eluting with 60% EtOAc/PE to afford Intermediate-35 (3.7 g, 32%) as yellow solid.

### Intermediate 38

### N-(2,3-dihydro-1H-inden-5-yl)acetamide (2)

To a stirring solution of SM1 (10 g, 75.2 mmol) and Et₃N (9.8 g, 97.7 mmol) in DCM (200 mL) was added drop-wise AcCl (6.5 g, 82.7 mmol) at 0° C. The reaction mixture was stirred at room temperature for 2 hours. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with MeOH/DCM to afford compound 2 (11.5 g, 90%) as a yellow solid. LC-MS: m/z=176 [M+H]⁺

### N-(1-oxo-2,3-dihydro-1H-inden-5-yl)acetamide (Intermediate 38)

To a stirring solution of compound 2 (11.5 g, 65.7 mmol) in AcOH: AcOAc (1:1, 200 ml) was added drop-wise CrO₃ (15 g, 151.11 mmol) dissolved in water, at 0° C. The reaction mixture was stirred at R T for 3 hours. The reaction mixture was diluted with water and extracted with EtOAc. Combined organic extracts were dried over anhydrous Na₂SO₄ and concentrated under reduced pressure to obtain crude product, which was purified by silica gel column chromatography eluting with MeOH/DCM to afford Intermediate-38 (9 g, 85%) as a yellow solid. LC-MS: m/z=190 [M+H]⁺

### Intermediate 39

### 5-amino-2,3-dihydrospiro[indene-1,5′-ozazolidine]-2′,4′-dione (Intermediate 39)

To a stirring suspension of intermediate 8 (29.5 g, 113 mmol) in CH₃OH (300 mL) was added concentrated HCl aqueous solution (75 mL). The resulting mixture was heated to reflux for 3 h. The precipitate was filtered and dried to afford intermediate 39 (16 g, 65%) as a yellow solid. LC-MS: m/z=219.1 [M+H]⁺

### Intermediate 40

### 2-(5-amino-2′,4′-dioxo-2,3-dihydrospiro[indene-1,5′-ozazolidine]-3′-yl)-N—((S)-1-cyclopropylethyl)-N-(4-fluorobenzyl)acetamide (Intermediate 40)

To a solution of intermediate 39 (0.66 g, 3 mmol) and intermediate 44 (0.94 g, 3 mmol) in DMF (20 mL) was added K₂CO₃ (1.24 g, 9 mmol). After stirring at room temperature for 2 h, the reaction mixture was diluted with water, extracted with EtOAc, washed with water and brine, dried and concentrated under reduced pressure to give the crude product which was purified by silica gel (methanol:DCM=1:20) to afford intermediate 40 (0.6 g, 44%) as a white solid. LC-MS: m/z=452.1 [M+H]⁺

### Intermediate 41

### (S,E)-N-(cyclopropylmethylene)-2-methylpropane-2-sulfinamide (1)

To a stirring solution of cyclopropanecarbaldehyde (2.8 g, 40 mmol) and (S)-2-methylpropane-2-sulfinamide (2.42 g, 20 mmol) in dry DCM (50 mL) were added PPTS (251 mg, 1 mmol) and MgSO₄ (12 g, 100 mmol). The resulting mixture was stirred at RT overnight. The solid was filtered off and the filtration was concentrated and purified by silica gel (petroleum ether:ethyl acetate=1:1) to give compound 1 (3.1 g, 86%) as oil. LC-MS: m/z=174.1 [M+H]⁺

### (S)—N—((R)-1-cyclopropyl-2,2,2-trifluoroethyl)-2-methylpropane-2-sulfinamide (2)

To a stirring solution of compound 1 (3.1 g, 18 mmol) and TMAF (4.45 g, 27 mmol) in dry THF (50 mL) at −55° C. under N₂ was added TMSCF₃ (3.8 g, 27 mmol). The reaction mixture was stirred at room temperature overnight, quenched with water, extracted with EtOAc, concentrated under reduced pressure to obtain the crude product which was purified by silica gel (methanol:DCM=1:20) to give compound 2 (1.9 g, 30%) as a white solid. LC-MS: m/z=244.0 [M+H]⁺

### (R)-1-cyclopropyl-2,2,2-trifluoroethanamine hydrochloride (3)

To a solution of compound 2 (1.9 g, 7.8 mmol) in MeOH (10 mL) was added HCl (2N in dioxane, 16 mL). After stirring at RT for 2 h, the reaction mixture was concentrated and the resulting solid was washed with diethyl ether to give compound 3 (1.2 g, 88%) as a white solid. LC-MS: m/z=140.1 [M+H]⁺

### (R)—N-benzyl-1-cyclopropyl-2,2,2-trifluoroethanamine (Intermediate 41)

To a solution of compound 3 (350 mg, 2 mmol) and (bromomethyl)benzene (513 mg, 3 mmol) in MeCN (10 mL) was added K₂CO₃ (828 mg, 6 mmol). The reaction mixture was stirred at room temperature overnight and concentrated under reduced pressure and purified by silica gel (methanol:DCM=1:20) to give intermediate 41 (0.23 g, 50%) as a off-white solid. LC-MS: m/z=230.1 [M+H]⁺

### Intermediate 42

### 2-(5-amino-2′,4′-dioxo-2,3-dihydrospiro[indene-1,5′-ozazolidine]-3′-yl)-N-(4-fluorobenzyl)-N—((S)-1,1,1-trifluoropropan-2-yl)acetamide (Intermediate 42)

To a solution of intermediate 39 (27.7 g, 127 mmol) and intermediate 46 (43.5 g, 127 mmol) in DMF (200 mL) was added K₂CO₃ (52.6 g, 381 mmol). The reaction mixture was then stirred at RT for 2 h, quenched with water, extracted with EtOAc, washed with water and brine, dried, concentrated under reduced pressure, and purified by silica gel (methanol:DCM=1:20) to afford intermediate 42 (43.9 g, 72%) as a white solid. LC-MS: m/z=480.0 [M+H]⁺

### Intermediate 43

### (R)-2-((tert-butoxycarbonyl)amino)-3-methylbutanoic acid (1)

To a solution of (R)-2-amino-3-methylbutanoic acid (1.17 g, 10 mmol) in THF (10 mL) at room temperature was added a solution of NaOH (400 mg, 10 mmol) in H₂O (10 mL), followed by (Boc)₂O (2.18 g, 10 mmol). After stirring for 2 h, the reaction mixture was adjusted to pH 3-4 with 6N HCl_{aq} and extracted with DCM. The organic phases were combined, dried over anhydrous Na₂SO₄, concentrated under reduced pressure and purified by silica gel column chromatography eluting with 5% CH₃OH in CH₂Cl₂ to afford compound 1 (2 g, 92%) as colorless oil.

### tert-butyl ((2R)-1-((2′,4′-dioxo-2,3-dihydrospiro[indene-1,5′-ozazolidin]-5-yl)amino)-3-methyl-1-oxobutan-2-yl)carbamate (Intermediate 43)

To a stirring solution of intermediate 39 (900 mg, 4.13 mmol) and compound 1 (1.34 g, 6.19 mmol) in DCM (40 mL) at room temperature was added Et₃N (838 mg, 8.26 mmol), followed by T₃P (50% in EtOAc) (5.25 g, 8.26 mmol). After stirring for 1 h, the reaction mixture was diluted with water and extracted with DCM. Combined organic extracts were dried over anhydrous Na₂SO₄, concentrated under reduced pressure and purified by silica gel column chromatography eluting with 5% MeOH in DCM to afford Intermediate 43 (1.03 g, 60%) as a light yellow solid. LC-MS: m/z=440.2 [M+Na]⁺

### Intermediate 44

### (S)-1-cyclopropyl-N-(4-fluorobenzyl)ethanamine (1)

To a stirring solution of 4-fluorobenzaldehyde (3.7 g, 30 mmol) and (S)-1-cyclopropylethanamine (2.1 g, 25 mmol) in MeOH (50 mL) was added Sodium triacetoxyborohydride (7.9 g, 37 mmol). The resulting reaction mixture was stirred at RT for 16 h. The reaction mixture was concentrated, diluted with EtOAc and washed with water and brine, dried, evaporated and purified by silica gel (methanol:DCM=1:20) to give compound 1 (4.5 g, 79%) as oil. LC-MS: m/z=194.1 [M+H]⁺

### (S)-2-bromo-N-(1-cyclopropylethyl)-N-(4-fluorobenzyl)acetamide (Intermediate 44)

To a stirring solution of compound 1 (4.5 g, 23 mmol) in dry DCM (20 mL) was added 2-bromoacetyl bromide (5.7 g, 28 mmol). The resulting mixture was stirred at RT for 1 h, concentrated and then purified by silica gel (petroleum ether:ethyl acetate=1:1) to give intermediate 44 (2.2 g, 67%) as oil. LC-MS: m/z=314.1/316.1 [M+H]⁺

### Intermediate 45

### (S,E)-N-(cyclopropylmethylene)-2-methylpropane-2-sulfinamide (1)

To a stirring solution of cyclopropanecarbaldehyde (2.8 g, 40 mmol) and (S)-2-methylpropane-2-sulfinamide (2.42 g, 20 mmol) in dry DCM (50 mL) were added PPTS (251 mg, 1 mmol) and MgSO₄ (12 g, 100 mmol). The resulting reaction mixture was stirred at RT for overnight. The solid was filtered off and the filtration was concentrated and then purified by silica gel (petroleum ether:ethyl acetate=1:1) to give compound 1 (3.1 g, 86%) as oil. LC-MS: m/z=174.1 [M+H]⁺

### (S)—N—((R)-1-cyclopropyl-2,2,2-trifluoroethyl)-2-methylpropane-2-sulfinamide (2)

To a stirring solution of compound 1 (3.1 g, 18 mmol) and TMAF (4.45 g, 27 mmol) in dry THF (50 mL) at −55° C. under N₂ was added TMSCF₃ (3.8 g, 27 mmol). The reaction mixture was stirred at room temperature for overnight, quenched with water, extracted with EtOAc, concentrated under reduced pressure and purified by silica gel (methanol:DCM=1:20) to give compound 2 (1.9 g, 30%) as a white solid. LC-MS: m/z=244.0 [M+H]⁺

### (R)-1-cyclopropyl-2,2,2-trifluoroethanamine hydrochloride (3)

To a solution of compound 2 (1.9 g, 7.8 mmol) in MeOH (10 mL) was added HCl (2N in dioxane, 16 mL). After stirring at RT for 2 h, the reaction mixture was evaporated and the resulting solid was washed with diethyl ether to give compound 3 (1.2 g, 88%) as a white solid. LC-MS: m/z=140.1 [M+H]⁺

### (R)-1-cyclopropyl-2,2,2-trifluoro-N-(4-fluorobenzyl)ethanamine (4)

To a solution of compound 3 (3.5 g, 20 mmol) and 1-(bromomethyl)-4-fluorobenzene (5.13 g, 30 mmol) in DMF (50 mL) was added K₂CO₃ (11 g, 80 mmol). The reaction mixture was stirred at room temperature for overnight, quenched with water, extracted with EtOAc, washed with water and brine, dried, concentrated under reduced pressure and purified by silica gel (methanol:DCM=1:20) to afford compound 4 (3.2 g, 65%) as oil. LC-MS: m/z=248.0 [M+H]⁺

### (R)-2-bromo-N-(1-cyclopropyl-2,2,2-trifluoroethyl)-N-(4-fluorobenzyl)acetamide (Intermediate 45)

To a stirring solution of compound 4 (3.2 g, 13 mmol) in dry DCM (50 mL) at 0° C. was added 2-bromoacetyl bromide (5.25 g, 26 mmol). After 1 h, the reaction mixture was quenched with NaHCO₃, extracted with DCM, dried, concentrated and purified by silica gel (petroleum ether:ethyl acetate=1:1) to give intermediate 45 (4.56 g, 95%) as oil. LC-MS: m/z 368.0/370.0 [M+H]⁺

### Intermediate 46

### (S)-1,1,1-trifluoro-N-(4-fluorobenzyl)propan-2-amine (1)

To a solution of (S)-1,1,1-trifluoropropan-2-amine (37 g, 327 mmol) and 1-(bromomethyl)-4-fluorobenzene (74.3 g, 393 mmol) in DMF (500 mL) was added K₂CO₃ (135 g, 981 mmol). The reaction mixture was stirred at room temperature for overnight, quenched with water, extracted with EtOAc, washed with water and brine, dried and concentrated under reduced pressure. The crude product was purified by silica gel (methanol:DCM=1:20) to afford compound 1 (55 g, 76%) as oil. LC-MS: m/z=222.0 [M+H]⁺

### (S)-2-bromo-N-(4-fluorobenzyl)-N-(1,1,1-trifluoropropan-2-yl)acetamide (Intermediate 46)

To a stirring solution of compound 1 (38.6 g, 175 mmol) in dry DCM (500 mL) was added 2-bromoacetyl bromide (70.4 g, 350 mmol). The resulting mixture was stirred at RT for 2 h, quenched with NaHCO₃, extracted with DCM, dried and concentrated and purified by silica gel (petroleum ether:ethyl acetate=1:1) to give intermediate 46 (49.5 g, 83%) as oil. LC-MS: m/z 342.0/344.0 [M+H]⁺

### Intermediate 47

### (R)—N-benzyl-2-bromo-N-(1-cyclopropyl-2,2,2-trifluoroethyl)acetamide (1)

To a stirring solution of intermediate 41 (180 mg, 0.78 mmol) in dry DCM (20 mL) were added pyridine (123 mg, 1.56 mmol) and 2-bromoacetyl bromide (235 mg, 1.17 mmol). The resulting reaction mixture was stirred at RT for 1 h, followed by concentration and purification by silica gel (petroleum ether:ethyl acetate=1:1) to give compound 1 (55 mg, 20%) as oil. LC-MS: m/z=349.9 [M+H]⁺

### 2-(5-amino-2′,4′-dioxo-2,3-dihydrospiro[indene-1,5′-ozazolidine]-3′-yl)-N-benzyl-N—((R)-1-cyclopropyl-2,2,2-trifluoroethyl)acetamide (Intermediate 47)

To a solution of compound 1 (75 mg, 0.21 mmol) and intermediate 39 (52 mg, 0.24 mmol) in DMF (5 mL) was added K₂CO₃ (58 mg, 0.42 mmol). The reaction mixture was stirred at room temperature for 1 h, quenched with water, extracted with EtOAc, washed with water and brine, dried and concentrated under reduced pressure. The crude product was then purified by silica gel (methanol:DCM=1:20) to give intermediate 47 (85 mg, 83%) as a brown solid. LC-MS: m/z=487.9 [M+H]⁺

### Intermediate 48

### 1-(tert-butoxycarbonyl)azetidine-3-carboxylic acid (1)

To a solution of (Boc)₂O (5.1 g, 23.4 mmol) in CH₃OH (10 mL) at room temperature was added a suspension of azetidine-3-carboxylic acid (2.02 g, 20 mmol) and Et₃N (2.02 mL, 20 mmol) in CH₃OH (40 mL). After stirring overnight, the reaction mixture was concentrated. The residue was diluted with H₂O, adjusted to pH 9-10 and extracted with DCM. The water layer was adjusted to pH to 2 with 4N HCl_{aq} and extracted with DCM. The combined organic extract was dried over anhydrous Na₂SO₄ and concentrated under reduced pressure to give the crude compound 1 (2.6 g, 64%) as a white solid.

### tert-butyl-(2′,4′-dioxo-2,3-dihydrospiro[indene-1,5′-ozazolidine]-5-ylcarbamoyl)azetidine-1-carboxylate (Intermediate 48)

To a solution of intermediate 39 (1.016 g, 4 mmol) and compound 1 (1.6 g, 8 mmol) in DCM (50 mL) was added Et₃N (1.2 mL, 8 mmol), followed by T₃P (50% in EtOAc) (5 mL, 8 mmol). After stirring at RT for 1 h, the reaction mixture was diluted with water and extracted with DCM. The combined organic extract was dried over anhydrous Na₂SO₄ and concentrated under reduced pressure. The crude product was then purified by silica gel column chromatography eluting with 5% MeOH in DCM to afford intermediate 48 (1.48 g, 92%) as an off-white solid. LC-MS: m/z=703.3 [2M+H-BOC]⁺

### Intermediate 49

### (R)—N-benzyl-2-bromo-N-(1-cyclopropyl-2,2,2-trifluoroethyl)acetamide (Intermediate 49)

To a solution of intermediate 41 (3.02 g, 13.2 mmol) in dry DCM (50 mL) was added bromoacetyl bromide (5.3 g, 26.4 mmol). After stirring at RT for 2 h, the mixture was quenched with a solution of NaHCO₃, extracted with DCM and concentrated under reduced pressure. The crude product was then purified by silica gel (petroleum ether:ethyl acetate=1:1) to give intermediate 49 (3.5 g, 75%) as a white solid. LC-MS: m/z 349.1/351.1 [M+H]⁺

### Intermediate 50

### tert-butyl 2-(5-amino-2′,4′-dioxo-2,3-dihydrospiro[indene-1,5′-ozazolidine]-3′-yl)acetate (1)

To a solution of intermediate 39 (100 mg, 0.45 mmol) and tert-butyl 2-bromoacetate (105 mg, 0.54 mmol) in DMF (5 mL) was added K₂CO₃ (124 mg, 0.9 mmol). The reaction mixture was stirred at room temperature for 1 h, quenched with water, extracted with EtOAc, washed with water and brine, dried and concentrated under reduced pressure. The crude product was purified by silica gel (methanol:DCM=1:20) to give compound 1 (100 mg, 66%) as a white solid. LC-MS: m/z=333.1 [M+H]⁺

### 2-(5-(3-methylureido)-2′,4′-dioxo-2,3-dihydrospiro[indene-1,5′-ozazolidine]-3′-yl)acetic acid (Intermediate 50)

To a solution of compound 1 (100 mg, 0.3 mmol) in THF (10 mL) at 0° C. under N₂ were added TEA (181 mg, 1.8 mmol) and triphosgen (45 mg, 0.15 mmol). After warming up to room temperature and stirring for 0.5 h, MeNH₂ (0.75 mL, 1.5 mmol) was added and the resulting mixture was stirred for additional one hour. The mixture was then quenched with water, extracted with EtOAc, dried and concentrated under reduced pressure. The crude product was purified by silica gel (methanol:DCM=1:20) to give an off-white solid. The solid was then dissolved in MeOH (10 mmol) and a concentrated HCl solution (2 mL) was added. After stirring at 60° C. for 5 h, the mixture was diluted with water, extracted with EtOAc, dried and concentrated to give intermediate 50 (50 mg, 50%) as a white solid. LC-MS: m/z=334.1 [M+H]⁺

### Intermediate 51

### (S)—N-(4-bromobenzyl)-1-cyclopropylethanamine (1)

A solution of 4-bromobenzaldehyde (1.18 g, 6.38 mmol) and (S)-1-cyclopropylethanamine (500 mg, 5.8 mmol) in MeOH (10 mL) was stirred at RT for 2 h. Then Sodium triacetoxyborohydride (1.47 g, 6.96 mmol) was added and stirring was continued at RT overnight. After quenching with water, the mixture was extracted with EtOAc, concentrated under reduced pressure and was purified by silica gel (methanol:DCM=1:20) to give compound 1 (150 mg, 10%). LC-MS: m/z 254.0/256.0 [M+H]⁺

### (S)-2-bromo-N-(4-bromobenzyl)-N-(1-cyclopropylethyl)acetamide (Intermediate 51)

To a stirring solution of compound 1 (0.15 g, 0.6 mmol) in dry DCM (20 mL) at room temperature was added bromoacetyl bromide (182 mg, 0.9 mmol). After 3 h, the mixture was diluted with water, extracted with DCM and concentrated under reduced pressure. The crude product was then purified by silica gel (petroleum ether:ethyl acetate=1:1) to give intermediate 51 (0.18 g, 81%). LC-MS: m/z 375.1 [M+H]⁺

### Intermediate 52

### (S)-2-(tert-butoxycarbonylamino)-3-methylbutanoic acid (1)

To a solution of (S)-2-amino-3-methylbutanoic acid (1.17 g, 10 mmol) and (Boc)₂O (2.18 g, 10 mmol) in THF (25 mL) and H₂O (25 mL) was NaOH (0.4 g, 10 mmol). After stirring at RT for 16 h, the mixture was diluted with water and extracted with EtOAc. The aqueous layer was adjusted to PH 2-3 with HCl (2N), extracted with EtOAc, dried and concentrated under reduced pressure to give compound 1 (2.2 g, 100%). LC-MS: m/z=117.1 [M+H-Boc]⁺

### tert-butyl (2S)-1-(2′,4′-dioxo-2,3-dihydrospiro[indene-1,5′-ozazolidine]-5-ylamino)-3-methyl-1-oxobutan-2-ylcarbamate (Intermediate 52)

To a solution of intermediate 39 (440 mg, 2 mmol) and compound 1 (650 mg, 2 mmol) in DCM (20 mL) were added TEA (606 mg, 6 mmol) and T₃P (1.27 g, 4 mmol). The resulting reaction mixture was then stirred at RT for 2 h. It was then quenched with water, extracted with DCM, washed with water and brine, dried and concentrated under reduced pressure. The crude product was purified by silica gel (methanol:DCM=1:20) to give intermediate 52 (550 mg, 65%) as a white solid. LC-MS: m/z=318.1 [M+H-Boc]⁺

### Example 2

### N-benzyl-N-(1-cyclobutylethyl)-2-(2,5-dioxo-2′,3′-dihydrospiro[imidazolidine-4,1′-indene]-1-yl)acetamide

To a stirring solution of compound Intermediate-20 (274 mg, 1.45 mmol) in DMF (5 mL) was added compound Intermediate-11 (390 mg, 1.5 mmol) and DIPEA (935 mg, 7.25 mmol) at room temperature. To this added HATU (1.1 g, 2.9 mmol) at room temperature and the reaction mixture was stirred at room temperature for 1 hour. the reaction mixture was diluted with water and extracted with EtOAc. Combined organic extracts were dried over anhydrous Na₂SO₄ and concentrated under reduced pressure to obtain crude product, which was purified by silica gel column chromatography eluting with 33% EtOAc/Hexane to afford compound 2-1 (300 mg, 48%). as an off-white solid. TLC: 50% EtOAc/Hexane (Rf: 0.65). LC-MS: m/z=432.0 [M+H]⁺ (97% purity). ¹H NMR (400 MHz, DMSO-d6): δ 8.79 (d, J=21.0 Hz, 1H), 7.43-7.15 (m, 9H), 4.64-4.37 (m, 4H), 4.70-4.40 (d, J=16.6 Hz, 0.5H), 4.30-3.81 (m, 2H), 3.05 (dd, J=13.8, 6.8 Hz, 2H), 2.61-2.54 (m, 1H), 2.41 (d, J=7.4 Hz, 1H), 2.29-2.15 (m, 1H), 1.92 (s, 1H), 1.75-1.50 (m, 5H), 1.00-0.85 (m, 3H).

### Example 3

### 3′,4′-dihydro-2′H-spiro[imidazolidine-4,1′-naphthalene]-2,5-dione (1)

To a stirring solution of 3,4-dihydronaphthalen-1(2H)-one SM1 (4.1 g, 28 mmol) in water (50 mL) and EtOH (50 ml) was added (NH₄)₂CO₃ (27 g, 280 mmol) and KCN (5.5 g, 85 mmol) at room temperature. The reaction mixture was heated at 70° C. overnight. After being cooled to room temperature and concentrated, the residue was purified by silica gel column chromatography eluting with 50% EtOAc/Hexane to afford compound 1 (3.6 g, 60%). LC-MS: m/z=214.2 [M+H]⁺

### tert-butyl 2-(2,5-dioxo-3′,4′-dihydro-2′H-spiro[imidazolidine-4, 1′-naphthalene]-1-yl)acetate (2)

To a stirring solution of compound 1 (0.65 g, 3 mmol) in dry DMF (10 ml) was added K₂CO₃ (0.84 g, 6 mmol) and tert-butyl 2-bromoacetate (0.59 g, 3 mmol) at room temperature. The reaction mixture was heated at 90° C. for 0.5 hours. After being cooled to room temperature the reaction mixture was diluted with cold water and extracted with EtOAc. Combined organic extracts were dried over anhydrous Na₂SO₄ and concentrated under reduced pressure to obtain crude product, which was purified by silica gel column chromatography eluting with 25% EtOAc/Hexane to afford compound 2 (0.95 g, 95%). TLC: 50% EtOAc/Hexane (R_{f}: 0.4)

### 2-(2,5-dioxo-3′,4′-dihydro-2′H-spiro[imidazolidine-4,1′-naphthalene]-1-yl)acetic acid (3)

To a stirring solution of compound 2 (0.95 g, 2.9 mmol) in dry DCM (8 mL) was added TFA (3.3 g, 29 mmol) at room temperature. The reaction mixture was stirred at room temperature for 2 hours then concentrated under reduced pressure to afford compound 3 (1.1 g, crude). LC-MS: m/z=261.2 [M+H]⁺

To a stirring solution of compound 3 (150 mg, 0.88 mmol) in DMF (5 mL) was added intermediate 14 (240 mg, 1.5 mmol) and DIPEA (340 mg, 2.76 mmol) at room temperature. To this added HATU (850 mg, 1.76 mmol) at room temperature and the reaction mixture was stirred at room temperature for 1 hour. the reaction mixture was diluted with water and extracted with EtOAc. Combined organic extracts were dried over anhydrous Na₂SO₄ and concentrated under reduced pressure to obtain crude product, which was purified by silica gel column chromatography eluting with 33% EtOAc/Hexane to afford compound 3-1 (130 mg, 34%). as an off-white solid. TLC: 50% EtOAc/Hexane (Rf: 0.45). LC-MS: m/z=432 [M+H]⁺ (98% purity). ¹H NMR (300 MHz, DMSO-d6): δ 8.88 (d, J=14.5 Hz, 1H), 7.46-7.09 (m, 9H), 4.74 (s, 1H), 4.62 (s, 1H), 4.41 (d, J=7.2 Hz, 0.5H), 4.35-4.14 (m, 1H), 3.74 (s, 0.5H), 2.78 (s, 2H), 2.15-1.75 (m, 5H), 1.25-1.15 (m, 3H), 0.90-0.80 (m, 2H), 0.50-0.30 (d, J=34.4 Hz, 2H), 0.22 (s, 3H).

### Example 4

### spiro[chroman-4,4′-imidazolidine]-2′,5′-dione (2)

To a suspension of chroman-4-one 1 (9.8 g, 66.1 mmol) in EtOH (80 mL) and H₂O (80 mL) was added KCN (6.46 g, 99.2 mmol) and (NH₃)₂CO₃ (19.5 g, 198 mmol), the reaction mixture was stirred in steel tube at 80° C. for 48 h. Cooled to room temperature and the reaction mixture was diluted with cold water and extracted with EtOAc. Combined organic extracts were dried over anhydrous Na₂SO₄ and concentrated under reduced pressure to obtain crude product, which was washed with cool EtOAc to afford compound 2 (13.2 g, 91%) as white solid. LC-MS: m/z=219.1 [M+H]⁺ (95% purity)

### tert-butyl 2-(2′,5′-dioxospiro[chroman-4,4′-imidazolidine]-1′-yl)acetate (4)

To a mixture of spiro[chroman-4,4′-imidazolidine]-2′,5′-dione 2 (3.27 g, 15 mmol) and tert-butyl 2-bromoacetate 3 (2.92 g, 15 mmol) in DMF (30 mL) was added K₂CO₃ (4.14 g, 30 mmol) and stirred at 90° C. for 30 min. After consumption of the starting material (by TLC), cooled to room temperature and the reaction mixture was diluted with cold water and extracted with EtOAc. Combined organic extracts were dried over anhydrous Na₂SO₄ and concentrated under reduced pressure to obtain crude product, which was purified by silica gel column chromatography (EtOAc/Hexane 1:2) to afford compound 4 (3.78 g, 76%) as white solid. LC-MS: m/z=277.1 [M+H-t-Bu]⁺ (95% purity)

### 2-(2′,5′-dioxospiro[chroman-4,4′-imidazolidine]-1′-yl)acetic acid (5)

To a solution of tert-butyl 2-(2′,5′-dioxospiro[chroman-4,4′-imidazolidine]-1′-yl)acetate (3.78 g, 11.4 mmol) in DCM (20 mL) was added TFA (20 mL) and stirred at room temperature for 2 hours. After consumption of the starting material (by TLC) and concentrated under reduced pressure to obtain crude compound 5 (2.87 g, 91%) as brown solid. LC-MS: m/z=277.1 [M+H]⁺ (90% purity)

To a solution of 2-(2′,5′-dioxospiro[chroman-4,4′-imidazolidine]-1′-yl)acetic acid (552 mg, 2 mmol) and intermediate 14 (525 mg, 3 mmol) in DMF (15 mL) was added DIPEA (1.03 g, 8 mmol) under N₂ and stirred at room temperature for 15 min. Then HATU (1.14 g, 3 mmol) was added and stirred at room temperature for 1 hour. After consumption of the starting material (by TLC) diluted with cold water and extracted with EtOAc. Combined organic extracts were dried over anhydrous Na₂SO₄ and concentrated under reduced pressure to obtain crude product, which was purified by silica gel column chromatography (EtOAc/Hexane 1:1) to afford 4-1 (0.8 g, 92%) as white solid. The compound 4-1 (240 mg) was purified by Chiral-HPLC to give P1 (35 mg), P2 (42 mg), P3 (17 mg) and P4 (26 mg). P1: LC-MS: m/z=424.0 [M+H]⁺ at RT 4.55 (95.12% purity); P2: LC-MS: m/z=424.0 [M+H]⁺ at RT 4.55 (95.14% purity); P3: LC-MS: m/z=424.0 [M+H]⁺ at RT 4.55 (95.53% purity); P4: LC-MS: m/z=424.0 [M+H]⁺ at RT 4.55 (95.21% purity). P1: ¹H NMR (400 MHz, CDCl₃) δ 7.45-7.33 (m, 4H), 7.34-7.29 (m, 1H), 7.28-7.19 (m, 2H), 7.01-6.94 (m, 1H), 6.88 (d, J=8.2 Hz, 1H), 5.88 (s, 1H), 4.82-4.59 (m, 3H), 4.44-4.12 (m, 3H), 4.01 (dd, J=9.4, 6.9 Hz, 1H), 2.53 (ddd, J=14.0, 7.0, 2.8 Hz, 1H), 2.24 (ddd, J=13.8, 8.6, 3.3 Hz, 1H), 1.24 (dd, J=19.0, 6.8 Hz, 3H), 0.99-0.74 (m, 1H), 0.69-0.48 (m, 1H), 0.43-0.21 (m, 3H); P2: ¹H NMR (400 MHz, CDCl₃) δ 7.47-7.34 (m, 4H), 7.31 (d, J=4.1 Hz, 1H), 7.25 (d, J=8.7 Hz, 2H), 7.03-6.83 (m, 2H), 5.72 (s, 1H), 4.69 (d, J=8.7 Hz, 3H), 4.54-4.13 (m, 3H), 4.00 (dd, J=9.4, 6.9 Hz, 1H), 2.53 (dd, J=14.0, 4.2 Hz, 1H), 2.26 (dd, J=8.7, 3.3 Hz, 1H), 1.24 (dd, J=27.1, 6.7 Hz, 3H), 0.97-0.75 (m, 1H), 0.66-0.48 (m, 1H), 0.43-0.20 (m, 3H); P3: ¹H NMR (400 MHz, CDCl₃) δ 7.41 (d, J=4.3 Hz, 3H), 7.36 (dd, J=7.8, 1.3 Hz, 1H), 7.31 (d, J=4.1 Hz, 1H), 7.25 (d, J=9.8 Hz, 2H), 7.06-6.84 (m, 2H), 5.82 (s, 1H), 4.88-4.60 (m, 3H), 4.25 (dd, J=25.3, 9.0 Hz, 3H), 4.00 (dd, J=9.3, 6.8 Hz, 1H), 2.53 (ddd, J=14.1, 7.1, 2.8 Hz, 1H), 2.33-2.16 (m, 1H), 1.24 (dd, J=27.6, 6.7 Hz, 3H), 1.00-0.73 (m, 1H), 0.67-0.48 (m, 1H), 0.43-0.19 (m, 3H); P4: ¹H NMR (400 MHz, CDCl₃) δ 7.49-7.34 (m, 4H), 7.34-7.29 (m, 1H), 7.27-7.16 (m, 2H), 7.05-6.85 (m, 2H), 5.75 (s, 1H), 4.79-4.60 (m, 3H), 4.51-4.12 (m, 3H), 4.02 (dd, J=9.4, 6.9 Hz, 1H), 2.65-2.46 (m, 1H), 2.26 (dd, J=8.7, 3.3 Hz, 1H), 1.24 (dd, J=18.6, 6.8 Hz, 3H), 0.95-0.74 (m, 1H), 0.68-0.47 (m, 1H), 0.45-0.15 (m, 3H).

### Example 5

### spiro[imidazolidine-4,3′-indoline]-2,2′,5-trione (2)

To a suspension of indoline-2,3-dione 1 (22.06 g, 150 mmol) in EtOH (150 mL) and H₂O (150 mL) was added KCN (15 g, 230 mmol) and (NH₄)₂CO₃ (43.2 g, 450 mmol), the reaction mixture was stirred in steel tube at 80° C. for 48 h, was cooled to room temperature, was diluted with cold water and extracted with EtOAc. Combined organic extracts were dried over anhydrous Na₂SO₄ and concentrated under reduced pressure to obtain crude product, which was washed with cool EtOAc to afford compound 2 (4.6 g, 14%) as white solid. LC-MS: m/z 218.1 [M+H]⁺ (95% purity)

### tert-butyl 2-(2,2′,5-trioxospiro[imidazolidine-4,3′-indoline]-1-yl)acetate (4)

To a mixture of spiro[imidazolidine-4,3′-indoline]-2,2′,5-trione 2 (2 g, 9.2 mmol) and tert-butyl 2-bromoacetate 3 (1.81 g, 9.2 mmol) in MeCN (50 mL) was added DIPEA (2.37 g, 18.4 mmol) and stirred at room temperature for 6 h. After consumption of the starting material (by TLC), reduced pressure to obtain crude product, which was purified by silica gel column chromatography (EtOAc/Hexane 1:2) to afford compound 4 (1.75 g, 58%) as light yellow solid. ¹H NMR (300 MHz, DMSO-d₆) δ 10.97 (s, 1H), 9.10 (s, 1H), 7.34 (d, J=7.7 Hz, 1H), 7.21 (d, J=7.3 Hz, 1H), 7.09 (d, J=7.5 Hz, 1H), 6.94 (d, J=7.7 Hz, 1H), 4.15 (d, J=1.8 Hz, 2H), 1.42 (s, 9H).

### 2-(2,2′,5-trioxospiro[imidazolidine-4,3′-indoline]-1-yl)acetic acid (5)

To a solution of tert-butyl 2-(2,2′,5-trioxospiro[imidazolidine-4,3′-indoline]-1-yl)acetate 4 (1.75 g, 5.3 mmol) in DCM (25 mL) was added TFA (25 mL) and stirred at room temperature for 2 hours. After consumption of the starting material (by TLC) and concentrated under reduced pressure to obtain crude compound 5 (1.55 g, 107%) as light yellow solid. LC-MS: m/z 276.1 [M+H]⁺ (90% purity)

To a solution of 2-(2,2′,5-trioxospiro[imidazolidine-4,3′-indoline]-1-yl)acetic acid 5 (275 mg, 1 mmol) and intermediate 14 (175 mg, 1 mmol) in DMF (5 mL) was added DIPEA (387 mg, 3 mmol) under N₂ and stirred at room temperature for 15 min. Then HATU (456 mg, 1.2 mmol) was added and stirred at room temperature for 1 hour. After consumption of the starting material (by TLC) diluted with cold water and extracted with EtOAc. Combined organic extracts were dried over anhydrous Na₂SO₄ and concentrated under reduced pressure to obtain crude product, which was purified by silica gel column chromatography (EtOAc/Hexane 1:1) to afford 5-1 (100 mg, 23%) as white solid. LC-MS: m/z=433.2 [M+H] at RT 4.09 (99.10% purity). ¹H NMR (300 MHz, DMSO-d6) δ 10.93 (s, 1H), 9.01 (d, J=7.2 Hz, 1H), 7.66-7.14 (m, 7H), 7.12-7.01 (m, 1H), 6.93 (d, J=7.7 Hz, 1H), 4.74-4.24 (m, 4H), 3.70 (d, J=6.0 Hz, 1H), 1.14 (dd, J=21.6, 4.9 Hz, 3H), 0.94 (s, 1H), 0.53-0.06 (m, 4H).

### Example 6

### N-(4-bromobenzyl)-1-cyclopropylethanamine (1)

To a stirring solution of (4-bromophenyl)methanamine SM1 (0.93 g, 5 mmol) and 1-cyclopropylethanone SM2 (420 mg, 5 mmol) in CH₃OH (25 mL) was added Ti(OiPr)₄ (1.56 g, 5.5 mmol) followed by NaBH₄ (380 mg, 10 mmol) at room temperature. The reaction mixture was stirred at room temperature for 16 h. After consumption of the starting material (by TLC), the reaction mixture was filtered. The filtrate was diluted with water and extracted with EtOAc. Combined organic extracts were dried over anhydrous Na₂SO₄ and concentrated under reduced pressure to obtain crude product, which was purified by silica gel column chromatography eluting with 20% PE/EtOAc to afford compound 1 (0.77 g, 50%) as a colorless oil. LC-MS: m/z=254.0/256.0 [M+H]⁺

To a stirring solution of compound 1 (200 mg, 0.77 mmol) and intermediate 20 (292 mg, 1.16 mmol) in DMF (5 mL) was added DIPEA (250 mg, 1.93 mmol) followed by HATU (585 mg, 1.54 mmol) and stirred at room temperature for 1 hour. The reaction mixture was diluted with water and extracted with DCM. Combined organic extracts were dried over anhydrous Na2SO4 and concentrated under reduced pressure to obtain crude product, which was purified by silica gel column chromatography eluting with 20% PE/EtOAc to afford 6-1 (160 mg, 49%) as an off-white solid. LC-MS: m/z=496.1/498.1 [M+H]⁺ at RT 8.53 (99.76% purity). ¹H NMR ((400 MHz, DMSO-d6) δ 8.81 (d, J=15.2 Hz, 1H), 7.49-7.20 (m, 6H), 7.17-6.95 (m, 2H), 4.78 (s, 1H), 4.63 (d, J=5.9 Hz, 1H), 4.50-4.12 (m, 2H), 3.76-3.72 (m, 0.5H), 3.43-3.39 (s, 0.5H), 3.05 (dd, J=11.8, 7.0 Hz, 2H), 2.61-2.52 (m, 1H), 2.22 (dd, J=13.4, 9.1 Hz, 1H), 1.26-1.03 (m, 3H), 1.01-0.84 (m, 1H), 0.53-0.08 (m, 4H).

### Example 7

### 1-cyclopropyl-N-(furan-2-ylmethyl)ethanamine (1)

To a stirring solution of 1-cyclopropylethanone SM1 (1.85 g, 22 mmol) in CH₃OH (20 mL) was added furan-2-ylmethanamine SM2 (2.14 g, 22 mmol) followed by Ti(OiPr)₄ (6.87 g, 24.2 mmol) at room temperature. The reaction mixture was stirred at room temperature for 4 h. Then NaBH₄ ((252 mg, 4 mmol) was added slowly and stirred at room temperature for 16 hours. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 10% EtOAc/PE to afford compound 1 (910 mg, 25%) as an colorless oil. LC-MS: m/z 166.1 [M+H]⁺

To a stirring solution of compound 1 (140 mg, 0.85 mmol) and intermediate 20 (220 mg, 0.85 mmol) in DCM (30 mL) was added Et₃N (172 mg, 1.69 mmol) followed by T3P (50% in EtOAc) (1.07 g, 1.69 mmol) and stirred at room temperature for 1 hour. The reaction mixture was diluted with water and extracted with DCM. Combined organic extracts were dried over anhydrous Na₂SO₄ and concentrated under reduced pressure to obtain crude product, which was purified by silica gel column chromatography eluting with 50% EtOAc/PE to afford compound 7-1 (190 mg, 55%) as an white solid. LC-MS: m/z=408.2 [M+H]⁺ at RT 4.27 (93.53% purity). ¹H NMR (400 MHz, DMSO-d6) δ 8.80 (d, J=9.6 Hz, 1H), 7.75-7.50 (m, 1H), 7.31 (m, 4H), 6.49-6.17 (m, 2H), 4.67 (d, J=27.3 Hz, 1H), 4.56-4.29 (m, 2H), 4.19 (dd, J=16.6, 5.4 Hz, 1H), 3.58 (d, J=7.5 Hz, 1H), 3.35 (s, 1H), 3.05 (t, J=6.8 Hz, 2H), 2.56 (dd, J=13.3, 6.6 Hz, 1H), 2.28-2.17 (m, 1H), 1.20 (d, J=5.3 Hz, 1H), 1.08 (s, 3H), 0.60-0.07 (m, 4H).

### Example 8

To a solution of 5-1 (110 mg, 0.25 mmol) and K₂CO₃ (103 mg, 0.75 mmol) in DMF (0.5 mL) and MeCN (5 mL) was added MeI (35.5 mg, 0.25 mmol) under N₂. Then stirred at room temperature for 2 hours. After consumption of the starting material (by TLC) diluted with cold water and extracted with EtOAc. Combined organic extracts were dried over anhydrous Na₂SO₄ and concentrated under reduced pressure to obtain crude product, which was purified by silica gel column chromatography (EtOAc/Hexane 2:3) to afford 8-1 (65 mg, 58%) as white solid. LC-MS: m/z=447.2 [M+H]⁺ at RT 4.33 (98.37% purity). ¹H NMR (400 MHz, DMSO-d6) δ 9.02 (d, J=9.7 Hz, 1H), 7.55-7.08 (m, 9H), 4.84-4.56 (m, 2H), 4.54-4.17 (m, 2H), 3.81-3.59 (m, 1H), 3.21 (d, J=5.6 Hz, 3H), 1.22-1.05 (m, 3H), 1.02-0.89 (m, 1H), 0.54-0.06 (m, 4H).

### Example 9

### 1-cyclopropyl-N-(thiophen-2-ylmethyl)ethanamine (3)

To a solution of thiophen-2-ylmethanamine 1 (2.26 g, 20 mmol) and 1-cyclopropylethanone 2 (1.68 g, 20 mmol) in MeOH (50 mL) was added titanium tetraisopropanolate (6.24 g, 22 mmol) and stirred at room temperature for 5 h. Then NaBH₄ (1.52 g, 40 mmol) was added and stirred at room temperature for overnight. Diluted with water and extracted with EtOAc. Combined organic extracts were dried over anhydrous MgSO₄ and concentrated under reduced pressure to obtain crude product, which was purified by silica gel column chromatography (EtOAc/Hexane 1:2) to afford 1-cyclopropyl-N-(thiophen-2-ylmethyl)ethanamine (3) (2.0 g, 55%) as oil. ¹H NMR (300 MHz, CDCl₃) δ 7.25-7.12 (m, 1H), 7.02-6.85 (m, 2H), 4.06 (d, J=0.7 Hz, 2H), 2.02-1.87 (m, 1H), 1.63 (s, 1H), 1.24-1.13 (m, 3H), 0.85-0.68 (m, 1H), 0.60-0.36 (m, 2H), 0.29-0.03 (m, 2H).

To a solution of intermediate 20 (260 mg, 1 mmol) and 1-cyclopropyl-N-(thiophen-2-ylmethyl)ethanamine 3 (271 mg, 1.5 mmol) in DMF (10 mL) was added DIPEA (516 mg, 4 mmol) under N₂ and stirred at room temperature for 15 min. Then HATU (570 mg, 1.5 mmol) was added and stirred at room temperature for 1 hour. After consumption of the starting material (by TLC) diluted with cold water and extracted with EtOAc. Combined organic extracts were dried over anhydrous Na₂SO₄ and concentrated under reduced pressure to obtain crude product, which was purified by silica gel column chromatography (EtOAc/Hexane 1:2) to afford compound 9-1 (260 mg, 61%) as white solid. LC-MS: m/z=424.0 [M+H]⁺ at RT 4.42 (99.29% purity). ¹H NMR (400 MHz, DMSO-d6) δ 8.80 (d, J=14.2 Hz, 1H), 7.60-6.81 (m, 7H), 4.90 (s, 1H), 4.73 (d, J=12.7 Hz, 1H), 4.41-4.10 (m, 2H), 3.77-3.50 (m, 1H), 3.05 (dd, J=14.8, 7.4 Hz, 2H), 2.55 (dd, J=6.8, 3.1 Hz, 1H), 2.22 (t, J=7.6 Hz, 1H), 1.24-1.00 (m, 4H), 0.63-0.10 (m, 4H).

### Example 10

### N-benzyl-N-(1-cyclopropylethyl)-2-(2,5-dioxo-2′,3′-dihydrospiro[imidazolidine-4,1′-indene]-1-yl)acetamide (1)

To a solution of intermediate 20 (130 mg, 0.5 mmol) and intermediate 14 (87 mg, 0.5 mmol) in DMF (10 mL) was added DIPEA (258 mg, 2 mmol) and stirred at room temperature for 15 mins. Then HATU (228 mg, 0.6 mmol) was added and stirred at room temperature for 1 hour. After consumption of the starting material (by TLC) diluted with water and extracted with EtOAc. Combined organic extracts were dried over anhydrous Na₂SO₄ and concentrated under reduced pressure to obtain crude product, which was purified by silica gel column chromatography (EtOAc/Hexane 1:2) to afford compound 1 (170 mg, 82%) as white solid. LC-MS: m/z=418.1 [M+H]⁺ (95% purity)

To a solution of N-benzyl-N-(1-cyclopropylethyl)-2-(2,5-dioxo-2′,3′-dihydrospiro[imidazolidine-4,1′-indene]-1-yl)acetamide 1 (170 mg, 0.4 mmol) and K₂CO₃ (166 mg, 1.2 mmol) in DMF (5 mL) was added MeI (113 mg, 0.8 mmol) under N₂ at room temperature. Then stirred at 60° C. for 1 hour. After consumption of the starting material (by TLC) diluted with cold water and extracted with EtOAc. Combined organic extracts were dried over anhydrous Na₂SO₄ and concentrated under reduced pressure to obtain crude product, which was purified by silica gel column chromatography (EtOAc/Hexane 1:2) to afford 10-1 (120 mg, 69%) as white solid. LC-MS: m/z=432.0 [M+H] at RT 4.92 (98.96% purity). ¹H NMR (400 MHz, DMSO-d6) δ 7.46-7.35 (m, 4H), 7.27 (dd, J=12.9, 6.2 Hz, 5H), 4.79-4.58 (m, 2H), 4.52-4.17 (m, 2H), 3.71 (d, J=2.8 Hz, 1H), 3.10 (t, J=7.2 Hz, 2H), 2.67-2.57 (m, 3H), 2.44 (dd, J=12.6, 6.8 Hz, 2H), 1.27-1.03 (m, 3H), 1.00-0.86 (m, 1H), 0.55-0.06 (m, 4H).

### Example 11

### 1-cyclopropyl-N-(4-fluorobenzyl)ethanamine (3)

To a solution of (4-fluorophenyl)methanamine 1 (1 g, 8 mmol) and 1-cyclopropylethanone 2 (670 mg, 8 mmol) in MeOH (20 mL) was added titanium tetraisopropanolate (2.5 g, 8.8 mmol) and stirred at room temperature for 4 h. Then NaBH₄ (0.61 g, 16 mmol) was added and stirred at room temperature for 2 hours. Diluted with water and extracted with DCM. Combined organic extracts were dried over anhydrous MgSO₄ and concentrated under reduced pressure to obtain crude product, which was purified by combiflash (EtOAc/Hexane 1:2) to afford 1-cyclopropyl-N-(4-fluorobenzyl)ethanamine (3) (210 mg, 14%) as oil. ¹H NMR (300 MHz, CDCl₃) δ 7.36-7.17 (m, 2H), 7.00 (td, J=8.6, 1.7 Hz, 2H), 3.81 (s, 2H), 1.98-1.74 (m, 2H), 1.20 (dd, J=6.3, 1.6 Hz, 3H), 0.86-0.68 (m, 1H), 0.63-0.31 (m, 2H), 0.26-0.02 (m, 2H).

To a solution of intermediate 20 (130 mg, 0.5 mmol) and 1-cyclopropyl-N-(4-fluorobenzyl)ethanamine 3 (87 mg, 0.5 mmol) in DMF (15 mL) was added DIPEA (258 mg, 2 mmol) under N₂ and stirred at room temperature for 15 min. Then HATU (285 mg, 0.75 mmol) was added and stirred at room temperature for 1 hour. After consumption of the starting material (by TLC) diluted with cold water and extracted with EtOAc. Combined organic extracts were dried over anhydrous Na₂SO₄ and concentrated under reduced pressure to obtain crude product, which was purified by silica gel column chromatography (EtOAc/Hexane 1:2) to afford compound 11-1 (130 mg, 60%) as white solid. LC-MS: m/z=436.0 [M+H]⁺ at RT 4.67 (98.32% purity). ¹H NMR (400 MHz, DMSO-d6) δ 8.80 (d, J=14.4 Hz, 1H), 7.44 (dd, J=8.5, 5.5 Hz, 1H), 7.40-7.15 (m, 6H), 7.10 (t, J=8.7 Hz, 1H), 4.73 (s, 1H), 4.60 (d, J=4.7 Hz, 1H), 4.50-4.10 (m, 2H), 3.78-3.65 (m, 1H), 3.05 (dd, J=13.3, 6.7 Hz, 2H), 2.61-2.52 (m, 1H), 2.22 (dd, J=13.4, 9.9 Hz, 1H), 1.29-1.04 (m, 3H), 0.93 (d, J=7.4 Hz, 1H), 0.61-0.08 (m, 4H).

### Example 12

### 1-cyclopropyl-N-(2-fluorobenzyl)ethanamine (1)

To a stirring solution of (2-fluorophenyl)methanamine SM1 (0.5 g, 4 mmol) and 1-cyclopropylethanone SM2 (336 mg, 4 mmol) in CH₃OH (25 mL) was added Ti(OiPr)₄ (1.25 g, 4.4 mmol) followed by NaBH₄ (304 mg, 8 mmol) at room temperature. The reaction mixture was stirred at room temperature for 16 h. After consumption of the starting material (by TLC), the reaction mixture was filtered. The filtrate was diluted with water and extracted with EtOAc. Combined organic extracts were dried over anhydrous Na₂SO₄ and concentrated under reduced pressure to obtain crude product, which was purified by silica gel column chromatography eluting with 20% EtOAc/Hexane to afford compound 1 (0.2 g, 26%) as a colorless oil. LC-MS: m/z=194.1 [M+H]⁺

To a stirring solution of compound 1 (149 mg, 0.77 mmol) and intermediate 20 (200 mg, 0.77 mmol) in DCM (30 mL) was added Et3N (156 mg, 1.54 mmol) followed by T3P (50% in EtOAc) (0.98 g, 1.54 mmol) and stirred at room temperature for 1 hour. The reaction mixture was diluted with water and extracted with DCM. Combined organic extracts were dried over anhydrous Na₂SO₄ and concentrated under reduced pressure to obtain crude product, which was purified by silica gel column chromatography eluting with 20% EtOAc/PE to afford compound 12-1 (210 mg, 62%) as an off-white solid. LC-MS: m/z=436.0 [M+H]⁺ at RT 4.69 (97.18% purity). ¹H NMR (400 MHz, DMSO-d6) δ 8.80 (d, J=13.0 Hz, 1H), 7.53-7.04 (m, 8H), 4.77 (s, 1H), 4.62 (s, 1H), 4.50-4.20 (m, 2H), 3.70 (d, J=10.7 Hz, 0.5H), 3.48-3.41 (m, 0.5H), 3.05 (t, J=6.9 Hz, 2H), 2.56 (dd, J=13.3, 6.4 Hz, 1H), 2.22 (dt, J=13.2, 6.5 Hz, 1H), 1.27-1.05 (m, 3H), 0.91 (dd, J=12.1, 4.7 Hz, 1H), 0.55-0.07 (m, 4H).

### Example 13

### 1-cyclopropyl-N-(3-fluorobenzyl)ethanamine (1)

To a stirring solution of (3-fluorophenyl)methanamine SM1 (0.5 g, 4 mmol) and 1-cyclopropylethanone SM2 (336 mg, 4 mmol) in CH₃OH (25 mL) was added Ti(OiPr)₄ (1.25 g, 4.4 mmol) followed by NaBH₄ (304 mg, 8 mmol) at room temperature. The reaction mixture was stirred at room temperature for 16 h. After consumption of the starting material (by TLC), the reaction mixture was filtered. The filtrate was diluted with water and extracted with EtOAc. Combined organic extracts were dried over anhydrous Na₂SO₄ and concentrated under reduced pressure to obtain crude product, which was purified by silica gel column chromatography eluting with 20% EtOAc/Hexane to afford compound 1 (0.25 g, 32%) as a colorless oil. LC-MS: m/z=194.1 [M+H]⁺

To a stirring solution of compound 1 (149 mg, 0.77 mmol) and intermediate 20 (200 mg, 0.77 mmol) in DCM (30 mL) was added Et₃N (156 mg, 1.54 mmol) followed by T3P (2,4,6-Tripropyl-1,3,5,2,4,6-Trioxatriphosphorinane-2,4,6-Trioxide)??What is this? (50% in EtOAc) (0.98 g, 1.54 mmol) and stirred at room temperature for 1 hour. The reaction mixture was diluted with water and extracted with DCM. Combined organic extracts were dried over anhydrous Na₂SO₄ and concentrated under reduced pressure to obtain crude product, which was purified by silica gel column chromatography eluting with 20% EtOAc/Hexane to afford compound 13-1 (35 mg, 10%) as an off-white solid. LC-MS: m/z=436.2 [M+H]⁺ at RT 4.62 (100% purity). ¹H NMR (400 MHz, DMSO-d6) δ 8.81 (d, J=15.2 Hz, 1H), 7.49-7.20 (m, 6H), 7.17-6.95 (m, 2H), 4.78 (s, 1H), 4.63 (d, J=5.9 Hz, 1H), 4.50-4.12 (m, 2H), 3.76-3.72 (m, 0.5H), 3.43-3.39 (s, 0.5H), 3.05 (dd, J=11.8, 7.0 Hz, 2H), 2.61-2.52 (m, 1H), 2.22 (dd, J=13.4, 9.1 Hz, 1H), 1.26-1.03 (m, 3H), 1.01-0.84 (m, 1H), 0.53-0.08 (m, 4H).

### Example 14

To a stirring solution of compound 1 (0.2 g, 0.59 mmol), intermediate 14 (114 mg, 0.65 mmol) and DIPEA (114 mg, 0.89 mmol) in DMF (4 mL) was added HATU (0.27 mg, 0.71 mmol). The mixture was stirred at room temperature for 1 hour. The mixture was diluted with water and extracted with EtOAc. Combined organic extracts were dried over anhydrous Na₂SO₄ and concentrated under reduced pressure. The residue was purified by Prep-HPLC to afford compound 19-1 (180 mg, 61%) as an off-white solid. LC-MS: m/z=496/498 [M+H]⁺ (100% purity). ¹H NMR (400 MHz, DMSO-d6): P1: δ 8.82 (s, 1H), 7.72-7.08 (m, 8H), 4.74 (s, 1H), 4.62 (s, 1H), 4.50-4.09 (m, 2H), 3.71 (dd, J=9.3, 6.8 Hz, 1H), 3.38 (d, J=7.5 Hz, 1H), 3.05 (dd, J=13.6, 6.8 Hz, 2H), 2.57 (dd, J=15.1, 6.8 Hz, 1H), 2.33-2.14 (m, 1H), 1.16 (ddd, J=19.1, 15.6, 9.5 Hz, 3H), 0.94 (s, 1H), 0.57-0.06 (m, 4H). ¹H NMR (400 MHz, DMSO-d6): P2:8.81 (s, 1H), 7.67-7.09 (m, 6H), 4.74 (s, 1H), 4.62 (d, J=6.4 Hz, 1H), 4.53-4.11 (m, 2H), 3.80-3.66 (m, 1H), 3.05 (dd, J=13.6, 6.6 Hz, 2H), 2.62-2.53 (m, 1H), 2.22 (dd, J=15.6, 9.4 Hz, 1H), 1.14 (dd, J=35.3, 6.7 Hz, 3H), 0.95 (s, 1H), 0.59-0.07 (m, 4H). ¹H NMR (400 MHz, DMSO-d6): P3: δ 8.83 (d, J=17.6 Hz, 1H), 7.66-7.11 (m, 6H), 4.74 (s, 1H), 4.62 (d, J=6.2 Hz, 1H), 4.51-4.13 (m, 2H), 3.73 (s, 1H), 3.38 (s, 1H), 3.13-2.94 (m, 2H), 2.55 (s, 1H), 2.21 (d, J=13.7 Hz, 2H), 1.14 (dd, J=35.3, 6.7 Hz, 3H), 0.95 (s, 1H), 0.59-0.07 (m, 4H). ¹H NMR (400 MHz, DMSO-d6): P4: δ 8.83 (d, J=17.9 Hz, 1H), 7.57 (s, 1H), 7.52-7.15 (m, 7H), 4.74 (s, 1H), 4.62 (s, 1H), 4.52-4.09 (m, 2H), 3.71 (dd, J=9.5, 6.8 Hz, 1H), 3.42-3.36 (m, 1H), 3.05 (dd, J=13.6, 6.7 Hz, 2H), 2.57 (d, J=8.5 Hz, 1H), 2.31-2.15 (m, 1H), 1.14 (dd, J=32.7, 6.7 Hz, 3H), 0.94 (s, 1H), 0.56-0.07 (m, 4H).

### Example 15

### 1-cyclopropyl-N-(furan-2-ylmethyl)ethanamine (3)

To a stirring solution of 1-cyclopropylethanamine 1 (0.5 g, 5.8 mmol) in MeOH (30 mL) was added furan-2-carbaldehyde (0.6 g, 7.6 mmol) followed by sodium triacetoxyborohydride (1.8 g, 10.5 mmol) at room temperature. The reaction mixture was stirred at room temperature overnight. After consumption of the starting material (by TLC), the reaction mixture was diluted with 1N NaOH and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 3 (0.5 g, 70%). LC-MS: m/z 166.1 [M+H]⁺

To a stirring solution of 4 (0.15 g, 0.44 mmol) in DMF (10 mL) was added compd-3 (73 mg, 0.44 mmol) and DIPEA (69 mg, 0.53 mmol) at room temperature and stirred for 2 mins, then HATU (0.18 g, 0.49 mmol) was added and the reaction mixture was stirred at room temperature for 1 hour. The solvent from the reaction was concentrated under reduced pressure to obtain crude product, which was purified by silica gel column chromatography to afford 15-1 (0.1 g, 54%) as an off-white solid. LC-MS: m/z=486.1/488.1 [M+H]⁺ at RT 1.63 (100% purity). ¹H NMR (400 MHz, DMSO) δ 8.84 (d, J=10.0 Hz, 1H), 7.66-7.49 (m, 3H), 7.24 (d, J=8.1 Hz, 1H), 6.52-6.21 (m, 2H), 4.82-4.59 (m, 2H), 4.46 (dt, J=31.1, 12.0 Hz, 2H), 4.35-4.13 (m, 1H), 3.57 (dd, J=16.2, 6.7 Hz, 1H), 3.05 (t, J=7.1 Hz, 2H), 2.56 (dd, J=13.5, 7.0 Hz, 1H), 2.30-2.16 (m, 1H), 1.28-0.99 (m, 4H), 0.51 (s, 1H), 0.51-0.07 (m, 4H), 0.07-0.02 (m, 1H).

### Example 16

### 5′-fluoro-2′,3′-dihydrospiro[imidazolidine-4,1′-indene]-2,5-dione (2)

To a stirring solution of compound 1 (2.0 g, 13.3 mmol) in 60% EtOH/H₂O (40 mL) was added (NH₄)₂CO₃ (6.4 g, 66.7 mol) followed by potassium cyanide (1.73 g, 26.6 mmol) at room temperature. The reaction mixture was heated to 70° C. for 16 h. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 2 (1.3 g, 67%). LC-MS: m/z=199.07 [M+H]⁺

### tert-butyl 2-(5′-fluoro-2,5-dioxo-2′,3′-dihydrospiro[imidazolidine-4,1′-indene]-1-yl)acetate (3)

To a stirring solution of compound 2 (1.0 g, 4.5 mmol) in DMF (20 mL) was added 2-bromoacetyl bromide (0.98 g, 5 mmol) and K₂CO₃ (1.38 g, 10 mmol) and stirred at room temperature overnight. After consumption of the starting material (by TLC), the reaction mixture was diluted with water and extracted with EtOAc. Combined organic extracts were dried over anhydrous Na₂SO₄ and concentrated under reduced pressure to obtain crude product 3 (1.27 g, 83%), which was used to the next step without any other purification. LC-MS: m/z=335.1 [M+H]⁺

### 2-(5′-fluoro-2,5-dioxo-2′,3′-dihydrospiro[imidazolidine-4,1′-indene]-1-yl)acetic acid (4)

To a stirring solution of compound 3 (1.9 g, 5.7 mmol) in DCM (4 mL) was added trifluoroacetic acid (4 mL) at 0° C. and stirred at room temperature for 0.5 h. The reaction mixture was concentrated under reduced pressure to obtain crude product, which was used to the next step without any other purification as compound 4 (1.7 g, 90%) as an off-white solid. LC-MS: m/z=279.1 [M+H]⁺

To a stirring solution of intermediate-14 (95 mg, 0.54 mmol) in DMF (10 mL) was added compd-4 (150 mg, 0.54 mmol) and DIPEA (84 mg, 0.65 mmol) at room temperature and stirred for 2 mins, then HATU (0.23 g, 0.59 mmol) was added and the reaction mixture was stirred at room temperature for 1 hour. The solvent from the reaction was concentrated under reduced pressure to obtain crude product, which was purified by silica gel column chromatography to afford 16-1 (0.11 g, 82%) as an off-white solid. LC-MS: m/z=436.1 [M+H]⁺ at RT 1.63 (100% purity). ¹H NMR (300 MHz, DMSO) δ 8.80 (d, J=12.9 Hz, 1H), 7.39 (t, J=6.0 Hz, 2H), 7.35-7.00 (m, 6H), 4.68-4.50 (m, 2H), 4.50-4.23 (m, 1H), 4.19 (s, 1H), 3.72 (d, J=8.3 Hz, 1H), 3.39 (d, J=16.1 Hz, 1H), 3.02 (t, J=5.5 Hz, 2H), 2.57 (dd, J=13.4, 6.6 Hz, 1H), 2.24 (dd, J=13.6, 7.9 Hz, 1H), 1.24-1.06 (m, 3H), 0.94 (d, J=3.7 Hz, 1H), 0.47 (s, 1H), 0.39-0.06 (m, 3H).

### Example 17

To a stirring solution of intermediate 31 (120 mg, 0.35 mmol) and intermediate 14 (61.3 mg, 0.35 mmol) was added HATU (1.2 eq) and DIPEA (2 ml). The resulted mixture was stirred at room temperature for 1 hour. The reaction mixture washed with water and extracted with EtOAc, the organic phase separated and dried with Na₂SO₄, concentrated in vacuum and pureed by Prep HPLC to afford 50 mg of desired product 17-1. yield: 42%. LC-MS: m/z=496/498 [M+H]⁺ at RT 1.68 (100.00% purity). ¹H NMR (400 MHz, DMSO) δ 8.88 (d, J=17.1 Hz, 1H), 7.58 (d, J=7.7 Hz, 1H), 7.47-7.15 (m, 7H), 4.81-4.14 (m, 4H), 3.38 (s, 1H), 3.02 (d, J=6.5 Hz, 2H), 2.63-2.53 (m, 1H), 2.32-2.19 (m, 1H), 1.23-1.03 (m, 3H), 0.94 (s, 1H), 0.56-0.06 (m, 4H).

### Example 18

### 1-cyclopropyl-N-(pyridin-3-ylmethyl)ethanamine (1)

A mixture of pyridin-3-ylmethanamine SM1 (0.5 g, 4.63 mmol), 1-cyclopropylethanone SM2 (0.39 g, 4.63 mmol) and tetraisopropyl titanate (1.45 g, 5.1 mmol) in MeOH (10 mL) was stirred under argon at room temperature for 4 h. NaBH₄ (0.35 g, 9.26 mmol) was then added at 0° C. and the resulting mixture was stirred for additional 2 hours. The mixture was quenched by adding water, stirring was maintained at room temperature for 20 min then the reaction mixture was acidified with HCl (a.q). After filtration washing with water and EtOAc, the organic layer was separated and the remaining aqueous layer was extracted once with EtOAc. The acidic aqueous extracts were treated with NaOH (a.q) to pH 10-12 and extracted with EtOAc. Combined organic extracts were dried over anhydrous Na₂SO₄ and concentrated under reduced pressure to obtain crude product, which was purified by silica gel column chromatography eluted with DCM/MeOH (50:1) to afford compound 1 (0.35 g, 43%) as an oil. LC-MS: m/z 177 [M+H]⁺

To a stirring solution of compound 2 (75 mg, 0.42 mmol), intermediate-20 (100 mg, 0.385 mmol) and DIPEA (100 mg, 0.77 mmol) in DMF (2 mL) was added HATU (293 mg, 0.77 mmol). The mixture was stirred at room temperature for 1 hour. The mixture was diluted with water and extracted with EtOAc. Combined organic extracts were washed with brine and dried over anhydrous Na₂SO₄, and concentrated under reduced pressure. The residue was purified by Prep-TLC to afford compound 18-1 (135 mg, 84%) as an off-white solid. LC-MS: m/z=419 [M+H]⁺ at RT 1.26 (100% purity). ¹H NMR (400 MHz, DMSO-d6): δ 8.85 (s, 1H), 8.74 (s, 1H), 8.34-8.18 (m, 1H), 7.89 (d, J=6.9 Hz, 1H), 7.47-7.18 (m, 4H), 4.98-4.66 (m, 2H), 4.58-4.12 (m, 2H), 3.76 (dt, J=9.4, 6.6 Hz, 1H), 3.51-3.37 (m, 1H), 3.05 (t, J=7.2 Hz, 2H), 2.61-2.54 (m, 1H), 2.30-2.16 (m, 1H), 1.21-1.00 (m, 3H), 0.98 (dd, J=8.3, 4.6 Hz, 1H), 0.59-0.03 (m, 4H).

### Example 19

### 1-cyclopropyl-N-(pyridin-2-ylmethyl)ethanamine (1)

A mixture of pyridin-2-ylmethanamine SM1 (0.5 g, 4.63 mmol), 1-cyclopropylethanone SM2 (0.39 g, 4.63 mmol) and tetraisopropyl titanate (1.45 g, 5.1 mmol) in MeOH (10 mL) was stirred under argon at room temperature for 4 h. NaBH₄ (0.35 g, 9.26 mmol) was then added at 0° C. and the resulting mixture was stirred for additional 2 hours. The mixture was quenched by adding water, stirring was maintained at room temperature for 20 min then the reaction mixture was acidified with HCl (a.q). After filtration washing with water and EtOAc, the organic layer was separated and the remaining aqueous layer was extracted once with EtOAc. The acidic aqueous extracts were treated with NaOH (a.q) to pH 10-12 and extracted with EtOAc. Combined organic extracts were dried over anhydrous Na₂SO₄ and concentrated under reduced pressure to obtain crude product, which was purified by silica gel column chromatography eluted with DCM/MeOH (50:1 to 20:1) to afford compound 1 (0.3 g, 37%) as an oil. LC-MS: m/z 177 [M+H]⁺

To a stirring solution of compound 2 (75 mg, 0.42 mmol), intermediate-20 (100 mg, 0.385 mmol) and DIPEA (100 mg, 0.77 mmol) in DMF (2 mL) was added HATU (293 mg, 0.77 mmol). The mixture was stirred at room temperature for 1 hour. The mixture was diluted with water and extracted with EtOAc. Combined organic extracts were washed with brine and dried over anhydrous Na₂SO₄, and concentrated under reduced pressure. The residue was purified by Prep-TLC to afford compound 19-1 (150 mg, 86%) as an off-white solid. LC-MS: m/z=419 [M+H]⁺ at RT 1.37 (100% purity). ¹H NMR (400 MHz, DMSO-d6): δ 8.80 (d, J=20.4 Hz, 1H), 8.72-8.54 (m, 1H), 7.88 (s, 1H), 7.64-7.17 (m, 5H), 4.83-4.75 (m, 2H), 4.55-4.28 (m, 2H), 3.75 (d, J=9.1 Hz, 1H), 3.54-3.43 (m, 1H), 3.04 (t, J=7.2 Hz, 2H), 2.62-2.54 (m, 1H), 2.28-2.15 (m, 1H), 1.27-1.05 (m, 3H), 0.94 (d, J=31.2 Hz, 1H), 0.57-0.03 (m, 4H).

### Example 20

### 5′-methoxy-2′,3′-dihydrospiro[imidazolidine-4,1′-indene]-2,5-dione (1)

To a stirring solution of SM1 (1.62 g, 10 mmol) in EtOH (17 mL), H₂O (17 mL) was added (NH₄)₂CO₃ (8.4 g, 87 mmol) followed by potassium cyanide (4.6 g, 70.7 mmol) at room temperature. The reaction mixture was heated to 75° C. for 64 h. After consumption of the starting material (by TLC), the reaction mixture was diluted with cold water and extracted with EtOAc. Combined organic extracts were dried over anhydrous Na₂SO₄ and concentrated under reduced pressure to obtain crude product, which was purified by silica gel column chromatography eluting with 50% EtOAc/PE to afford compound 1 (0.429 g, 19%) as a yellow solid. LC-MS: m/z=233 [M+H]⁺

### tert-butyl 2-(5′-methoxy-2,5-dioxo-2′,3′-dihydrospiro[imidazolidine-4,1′-indene]-1-yl)acetate (2)

To a stirring solution of compound 1 (429 mg, 1.85 mmol) in DMF (2 mL) was added K₂CO₃ (383 mg, 2.78 mmol) followed by tert-butyl 2-bromoacetate (378.8 mg, 1.943 mmol) at room temperature and stirred at room temperature for 1 hour. The reaction mixture was diluted with water and extracted with EtOAc. Combined organic extracts were dried over anhydrous Na₂SO₄ and concentrated under reduced pressure to obtain crude product, which was purified by silica gel column chromatography eluting with 50% EtOAc/PE to afford compound 2 (482 mg, 75%) as a yellow solid. LC-MS: m/z=291 [M+H-t-Bu]⁺

### 2-(5′-methoxy-2,5-dioxo-2′,3′-dihydro spiro[imidazolidine-4,1′-indene]-1-yl)acetic acid (3)

To a stirring solution of compound 2 (482 mg, 1.39 mmol) in DCM (4 mL) was added TFA (1 mL) at room temperature and stirred at room temperature for 1 hour. After consumption of the starting material (by TLC), the reaction mixture was concentrated under reduced pressure to obtain crude compound 3 (442 mg, 99%) as a yellow solid. LC-MS: m/z=291 [M+H]⁺

To a stirring solution of compound 3 (200 mg, 0.69 mmol) in DMF (2 mL) was added intermediate 14 (121 mg, 0.69 mmol) and DIPEA (133.5 mg, 1.04 mmol) followed by HATU (314.6 mg, 0.828 mmol) at room temperature and stirred at room temperature for 1 hour. The reaction mixture was diluted with water and extracted with EtOAc. Combined organic extracts were dried over anhydrous Na2SO4 and concentrated under reduced pressure to obtain crude product, which was purified by silica gel column chromatography eluting with 5% MeOH/DCM to afford 20-1 (166 mg, 54%) as a white solid. LC-MS: m/z=448.2 [M+H]⁺. ¹H NMR (400 MHz, DMSO-d6): δ 8.73 (d, J=16.6 Hz, 1H), 7.39 (q, J=8.1 Hz, 2H), 7.33-7.13 (m, 4H), 6.89 (s, 1H), 6.83 (dd, J=10.3, 4.2 Hz, 1H), 4.74 (s, 1H), 4.62 (s, 1H), 4.48-4.11 (m, 2H), 3.75 (d, J=2.7 Hz, 3H), 3.39 (d, J=7.3 Hz, 1H), 3.00 (d, J=6.5 Hz, 2H), 2.58 (d, J=7.5 Hz, 1H), 2.25-2.13 (m, 1H), 1.22-1.06 (m, 3H), 0.93 (d, J=9.5 Hz, 1H), 0.57-0.43 (m, 1H), 0.41-0.06 (m, 3H).

### Example 21

### 4′-methoxy-2′,3′-dihydrospiro[imidazolidine-4,1′-indene]-2,5-dione (1)

To a stirring solution of 4-methoxy-2,3-dihydroinden-1-one SM1 (1.62 g, 10 mmol) in 50% EtOH/H₂O (40 mL) was added (NH₄)₂CO₃ (20 g, 120 mmol) followed by potassium cyanide (2 g, 30 mmol) at room temperature. The reaction mixture was heated to 75° C. for 18 h. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 10% MeOH/DCM to afford compound 1 (1.3 g, 56%) as a white solid. LC-MS: m/z=233.1 [M+H]⁺

### tert-butyl 2-(4′-methoxy-2,5-dioxo-2′,3′-dihydrospiro[imidazolidine-4,1′-indene]-1-yl)acetate (2)

To a stirring solution of compound 1 (464 mg, 2 mmol) and tert-butyl 2-bromoacetate SM2 (429 mg, 2.2 mmol) in DMF (6 mL) was added K₂CO₃ (553 mg, 4 mmol) and stirred at room temperature for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 2 (567 mg, 82%) as an off-white solid. LC-MS: m/z=291.1 [M+H]⁺

### 2-(4′-methoxy-2,5-dioxo-2′,3′-dihydrospiro[imidazolidine-4,1′-indene]-1-yl)acetic acid (3)

To a stirring solution of compound 2 (567 mg, 1.64 mmol) in DCM (6 mL) was added TFA (3 mL) and stirred at room temperature for 2 hours. The reaction mixture was concentrated under reduced pressure to obtain compound 3 (320 mg, 67%) as an off-white solid used for next step directly. LC-MS: m/z=291.1 [M+H]⁺

To a stirring solution of compound 3 (320 mg, 1.1 mmol) and N-benzyl-1-cyclopropylethanamine intermediate 14 (193 mg, 1.1 mmol) in DCM (30 mL) was added Et₃N (223 mg, 2.2 mmol) followed by T3P (50% in EtOAc) (1.4 g, 2.2 mmol) and stirred at room temperature for 1 hour. The reaction mixture was diluted with water and extracted with DCM. Combined organic extracts were dried over anhydrous Na₂SO₄ and concentrated under reduced pressure to obtain crude product, which was purified by silica gel column chromatography eluting with 50% EtOAc/Hexane to afford compound 21-1 (110 mg, 22%) as an off-white solid. LC-MS: m/z=448.2 [M+H]⁺ at RT 4.62 (98.63% purity). ¹H NMR (400 MHz, DMSO-d6) δ 8.77 (d, J=16.5 Hz, 1H), 7.44-7.13 (m, 6H), 6.98-6.85 (m, 2H), 4.74 (s, 1H), 4.62 (s, 1H), 4.46-4.12 (m, 2H), 3.82 (s, 3H), 3.75-3.70 (m, 0.5H), 3.44-3.35 (m, 0.5H), 2.93 (dd, J=11.3, 6.0 Hz, 2H), 2.54 (d, J=8.5 Hz, 1H), 2.19 (dd, J=12.5, 7.8 Hz, 1H), 1.24-1.04 (m, 3H), 0.98-0.91 (m, 1H), 0.56-0.13 (m, 4H).

### Example 22

### 6,7-dihydro-5H-cyclopenta[b]pyridin-5-one (1)

To a stirring solution of 6,7-dihydro-5H-cyclopenta[b]pyridine SM1 (3.83 g, 32 mmol) in HOAc (20 mL) and conc. H₂SO₄ (3.5 ml) was added a solution of CrO₃ (6.7 g, 67.2 mmol) in 10 ml of HOAc and 2 mL of H₂O. The temperature was kept below 10° C. during the addition. After addition completed the resultant mixture was stirred at room temperature overnight. The mixture was then poured into crushed ice and treated with NH₄OH to pH 11 and extracted with DCM. The organic phase was worked up and purified by silica gel column chromatography eluting with 50% EtOAc/Hexane to afford compound 1 (1.1 g, 35%). LC-MS: m/z=134.0 [M+H]⁺

### 6,7-dihydrospiro[cyclopenta[b]pyridine-5,4′-imidazolidine]-2′,5′-dione (2)

To a stirring solution of compound 1 (1.1 g, 8.4 mmol) in water (20 mL) and EtOH (20 ml) was added (NH₄)₂CO₃ (8.2 g, 84 mmol) and KCN (5 g, 77 mmol) at room temperature. The reaction mixture was heated at 70° C. overnight. After being cooled to room temperature and concentrated to half volume, the mixture was extracted with EtOAc, the organic extracts were dried over anhydrous Na₂SO₄ and concentrated under reduced pressure to obtain crude compound 2 (1.1 g), which was used in next step without further purification. LC-MS: m/z=204.2 [M+H]⁺

### tert-butyl 2-(2′,5′-dioxo-6,7-dihydrospiro[cyclopenta[b]pyridine-5,4′-imidazolidine]-1′-yl)acetate (3)

To a stirring solution of crude compound 2 (1.1 g, 5.4 mmol) in dry DMF (10 ml) was added K₂CO₃ (1.5 g, 10.8 mmol) and tert-butyl 2-bromoacetate (1.17 g, 6 mmol) at room temperature. The reaction mixture was heated at room temperature for 1 hour. After being cooled to room temperature the reaction mixture was diluted with cold water and extracted with EtOAc. Combined organic extracts were dried over anhydrous Na₂SO₄ and concentrated under reduced pressure to obtain crude product, which was purified by silica gel column chromatography eluting with 70% EtOAc/Hexane to afford compound 3 (0.91 g, 52%). TLC: 80% EtOAc/Hexane (R_{f}: 0.3)

### 2-(2′,5′-dioxo-6,7-dihydrospiro[cyclopenta[b]pyridine-5,4′-imidazolidine]-1′-yl)acetic acid (4)

To a stirring solution of compound 3 (0.91 g, 2.8 mmol) in dry DCM (8 mL) was added TFA (3.3 g, 29 mmol) at room temperature. The reaction mixture was stirred at room temperature for 2 hours then concentrated under reduced pressure to afford compound 4 (0.8 g, crude). LC-MS: m/z=262.2 [M+H]⁺

To a stirring solution of compound 4 (100 mg, 0.38 mmol) in DMF (3 mL) was added compound intermediate 14 (68 mg, 0.38 mmol) and DIPEA (150 mg, 1.2 mmol) at room temperature. To this added HATU (300 mg, 0.77 mmol) at room temperature and the reaction mixture was stirred at room temperature for 1 hour. the reaction mixture was diluted with water and extracted with EtOAc. Combined organic extracts were dried over anhydrous Na₂SO₄ and concentrated under reduced pressure to obtain crude product, which was purified by Prep-TLC eluting with 10% MeOH/DCM to afford compound 22-1 (35 mg, 21%). as an off-white solid. TLC: 10% MeOH/DCM (Rf: 0.6). LC-MS: m/z=419 [M+H]⁺ (98% purity). ¹H NMR (400 MHz, DMSO-d6): δ 8.85 (d, J=13.1 Hz, 1H), 8.48 (d, J=4.6 Hz, 1H), 7.75-7.63 (m, 1H), 7.44-7.13 (m, 6H), 4.72 (s, 1H), 4.59 (s, 1H), 4.47-4.18 (m, 2H), 3.29 (s, 1H), 3.07 (dd, J=11.5, 7.3 Hz, 2H), 2.65-2.55 (m, 1H), 2.23 (dd, J=13.5, 8.0 Hz, 1H), 1.24-0.86 (m, 4H), 0.55-0.07 (m, 4H).

### Example 23

### tert-butyl 2-(5′-bromo-2,5-dioxo-2′,3′-dihydrospiro[imidazolidine-4,1′-indene]-1-yl)acetate (1)

To a stirring solution of interm-10 (1.0 g, 3.5 mmol) in DMF (20 mL) was added 2-bromoacetyl bromide (0.73 g, 3.7 mmol) and K₂CO₃ (1.0 g, 7.1 mmol) and stirred at room temperature overnight. After consumption of the starting material (by TLC), the reaction mixture was diluted with water and extracted with EtOAc. Combined organic extracts were dried over anhydrous Na₂SO₄ and concentrated under reduced pressure to obtain crude product 1 (1.6 g, 95%), which was used to the next step without any other purification. LC-MS: m/z=395.1/397.1 [M+H]⁺

### 2-(5′-bromo-2,5-dioxo-2′,3′-dihydrospiro[imidazolidine-4,1′-indene]-1-yl)acetic acid (2)

To a stirring solution of compound 1 (1.6 g, 4.0 mmol) in DCM (4 mL) was added trifluoroacetic acid (4 ml) at 0° C. and stirred at room temperature for 0.5 h. The reaction mixture was concentrated under reduced pressure to obtain crude product, which was used to the next step without any other purification as compound 2 (1.3 g, 90%) as an off-white solid. LC-MS: m/z 339.1/341.1 [M+H]⁺

### 2-(5′-cyano-2,5-dioxo-2′,3′-dihydrospiro[imidazolidine-4,1′-indene]-1-yl)acetic acid (3)

To a stirring solution of compound 2 (0.3 g, 0.9 mmol) in DMF (4 mL) was added Pd(PPh₃)₄ (0.1 g, 0.1 mmol) and Zn(CN)₂ (0.1 g, 0.9 mmol) and stirred at 160° C. under microwave for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product used to the next step without any other purification as compound 3 (0.12 g, 41%)

To a stirring solution of compd-3 (100 mg, 0.35 mmol) in DMF (10 mL) was added intermediate-14 (61 mg, 0.35 mmol) and DIPEA (55 mg, 0.43 mmol) at room temperature and stirred for 2 mins, then HATU (0.15 g, 0.38 mmol) was added and the reaction mixture was stirred at room temperature for 1 hour. The solvent from the reaction was concentrated under reduced pressure to obtain crude product, which was purified by silica gel column chromatography to afford 23-1 (15 mg, 18%) as an off-white solid. LC-MS: m/z=443.1 [M+H]⁺ at RT 1.68 (100% purity). ¹H NMR (400 MHz, DMSO) δ 8.92 (d, J=18.2 Hz, 1H), 7.86 (s, 1H), 7.79-7.77 (m, 1H), 7.57-7.44 (m, 1H), 7.47-7.32 (m, 2H), 7.25 (dt, J=18.9, 9.7 Hz, 3H), 4.85-4.56 (m, 2H), 4.53-4.24 (m, 1H), 4.26-4.11 (m, 1H), 3.73 (dd, J=16.6, 7.6 Hz, 1H), 3.10 (dd, J=13.8, 6.8 Hz, 2H), 2.69-2.55 (m, 1H), 2.27 (ddd, J=20.9, 13.0, 7.9 Hz, 1H), 1.30-1.04 (m, 3H), 0.93 (d, J=7.6 Hz, 1H), 0.49 (d, J=8.2 Hz, 1H), 0.42-0.06 (m, 3H).

### Example 24

### 1-cyclopropyl-N-(pyridin-4-ylmethyl)ethanamine (3)

To a solution of pyridin-4-ylmethanamine 1 (2.16 g, 20 mmol) and 1-cyclopropylethanone 2 (1.68 g, 20 mmol) in MeOH (50 mL) was added titanium tetraisopropanolate (6.24 g, 22 mmol) and stirred at room temperature for 4 h. Then NaBH₄ (1.52 g, 40 mmol) was added and stirred at room temperature for 3 hours. Diluted with water and extracted with DCM. Combined organic extracts were dried over anhydrous MgSO₄ and concentrated under reduced pressure to obtain crude product, which was purified by silica gel column chromatography (EtOAc/Hexane 1:2) to afford 1-cyclopropyl-N-(pyridin-4-ylmethyl)ethanamine (3) (1.2 g, 34%) as oil. ¹H NMR (300 MHz, CDCl₃) δ 8.52 (dd, J=4.4, 1.5 Hz, 2H), 7.26 (dd, J=3.2, 2.1 Hz, 2H), 3.87 (s, 2H), 1.99-1.63 (m, 2H), 1.18 (d, J=6.3 Hz, 3H), 0.75 (d, J=8.3 Hz, 1H), 0.65-0.37 (m, 2H), 0.30-0.00 (m, 2H). LC-MS: m/z=177.1 [M+H]⁺ (95% purity).

To a solution of intermediate 20 (130 mg, 0.5 mmol) and 1-cyclopropyl-N-(pyridin-4-ylmethyl)ethanamine 3 (88 mg, 0.5 mmol) in DMF (5 mL) was added DIPEA (258 mg, 2 mmol) under N2 and stirred at room temperature for 15 min. Then HATU (285 mg, 0.75 mmol) was added and stirred at room temperature for 1 hour. After consumption of the starting material (by TLC) diluted with cold water and extracted with EtOAc. Combined organic extracts were dried over anhydrous Na₂SO₄ and concentrated under reduced pressure to obtain crude product, which was purified by silica gel column chromatography (EtOAc/Hexane 1:2) to afford compound 24-1 (125 mg, 59%) as white solid. LC-MS: m/z=419.0 [M+H]⁺ at RT 4.95 (99.28% purity). ¹H NMR (300 MHz, DMSO-d6) δ 8.80 (d, J=8.7 Hz, 1H), 8.54-8.44 (m, 2H), 7.67-6.97 (m, 6H), 4.80-4.55 (m, 2H), 4.51-3.99 (m, 2H), 3.92-3.38 (m, 1H), 3.04 (t, J=6.9 Hz, 2H), 2.54 (s, 1H), 2.30-2.11 (m, 1H), 1.27-1.03 (m, 3H), 1.02-0.76 (m, 1H), 0.16 (d, J=5.9 Hz, 4H).

### Example 25

### 4′-chloro-2′,3′-dihydrospiro[imidazolidine-4,1′-indene]-2,5-dione (1)

A mixture of 4-chloro-2,3-dihydro-1H-inden-1-one compound 1 (1 g, 6 mmol), (NH₄)₂CO₃ (9.4 g, 60 mmol) and KCN (4 g, 61.5 mmol) in EtOH/H₂O (10 mL/10 mL) was heated at 60° C. for 15 h in a sealed vial. The reaction mixture was extracted with EtOAc. Combined organic extracts were dried over anhydrous Na₂SO₄ and concentrated under reduced pressure to obtain crude product, which was purified by flash chromatography on silica gel using a mixture of (PE/EtOAc 50/50) as eluent to afford compound 1 (0.8 g, 57%) as a solid. LC-MS: m/z=236/238 [M+H]⁺. tert-butyl 2-(4′-chloro-2,5-dioxo-2′,3′-dihydrospiro[imidazolidine-4,1′-indene]-1-yl)acetate (2):

A mixture of compound 1 (0.5 g, 2.1 mmol), tert-butyl 2-bromoacetate (453 mg, 2.32 mmol) and K₂CO₃ (435 mg, 3.15 mmol) was stirred at room temperature for 1 hour. The reaction mixture was diluted with water and extracted with EtOAc. Combined organic extracts were dried over anhydrous Na₂SO₄ and concentrated under reduced pressure. The residue was used to the next step without purification. LC-MS: m/z=350/352 [M+H]⁺

### 2-(4′-chloro-2,5-dioxo-2′,3′-dihydrospiro[imidazolidine-4,1′-indene]-1-yl)acetic acid (3)

To a solution of compound 2 (0.5 g, 2.1 mmol) in DCM (3 mL) was added TFA (3 mL). The mixture was stirred at room temperature for 1.5 h. The solvent was concentrated under reduced pressure. The residue was used to the next step without purification. LC-MS: m/z=294/296 [M+H]⁺

To a stirring solution of compound 3 (0.12 g, 0.4 mmol), intermediate-14 (77 mg, 0.44 mmol) and DIPEA (228 mg, 1.2 mmol) in DMF (3 mL) was added HATU (228 mg, 0.6 mmol). The mixture was stirred at room temperature for 1 hour. The mixture was diluted with water and extracted with EtOAc. Combined organic extracts were dried over anhydrous Na2SO4 and concentrated under reduced pressure. The residue was purified by Prep-TLC to afford compound 25-1 (40 mg, 22%) as an off-white solid. LC-MS: m/z=452/454 [M+H]⁺ at RT 1.67 (97.98% purity). ¹H NMR (400 MHz, DMSO-d6): δ 8.87 (d, J=17.1 Hz, 1H), 7.49-7.15 (m, 4H), 4.74 (s, 1H), 4.63 (d, J=5.2 Hz, 1H), 4.49-4.38 (m, 1H), 4.30 (dd, J=16.5, 3.7 Hz, 1H), 4.20 (d, J=3.8 Hz, 1H), 3.72 (d, J=9.5 Hz, 1H), 3.05 (dd, J=13.3, 6.2 Hz, 1H), 2.59 (dd, J=13.8, 5.5 Hz, 1H), 2.38-2.19 (m, 1H), 1.23-1.06 (m, 1H), 0.94 (s, 1H), 0.48 (d, J=3.6 Hz, 1H), 0.42-0.33 (m, 1H), 0.19 (ddd, J=16.3, 10.5, 5.4 Hz, 1H).

### Example 26

### N-benzylpropan-2-amine (1)

To a stirring solution of phenylmethanamine SM1 (1.07 g, 10.0 mmol) in DCM (20 mL) and was added Acetone (0.58 g, 10.0 mmol) and NaBH(OAc)₃ (3.18 g, 15 mmol) at room temperature. The reaction mixture was heated at 30° C. overnight. After being cooled to room temperature and concentrated, the residue was purified by silica gel column chromatography eluting with 50% EtOAc/Hexane to afford compound 1 (1.04 g, 70%). LC-MS: m/z=150.3 [M+H]⁺

To a stirring solution of intermediate 20 (80 mg, 0.31 mmol) and Compd-1 (46 mg, 0.31 mmol) was added HATU (1.2 eq) and DIPEA (2 ml). The resulted mixture was stirred at room temperature for 1 hour. The reaction mixture washed with water and extracted with EtOAc, the organic phase separated and dried with Na₂SO₄, concentrated in vacuum and pureed by Prep HPLC to afford 20 mg of desired product 26-1 yield: 17%.

LC-MS: m/z=392.2 [M+H]⁺ at RT 1.55 (100.00% purity). ¹H NMR (400 MHz, DMSO) δ 8.81 (d, J=12.7 Hz, 1H), 7.48-7.10 (m, 9H), 4.63 (s, 1H), 4.58 (dd, J=13.6, 6.8 Hz, 0.5H), 4.50 (d, J=17.6 Hz, 2H), 4.28 (dd, J=13.1, 6.5 Hz, 0.5H), 4.18 (s, 1H), 3.05 (dd, J=12.3, 6.9 Hz, 2H), 2.61-2.53 (m, 1H), 2.29-2.18 (m, 1H), 1.17-0.96 (m, 6H).

### Example 27

### N-benzylbutan-2-amine (1)

To a stirring solution of phenylmethanamine SM1 (1.07 g, 10.0 mmol) in DCM (20 mL) and was added butan-2-one (0.72 g, 10.0 mmol) and NaBH(OAc)₃ (3.18 g, 15 mmol) at room temperature. The reaction mixture was heated at 30° C. overnight. After being cooled to room temperature and concentrated, the residue was purified by silica gel column chromatography eluting with 50% EtOAc/Hexane to afford compound 1 (1.22 g, 75%). LC-MS: m/z=164.1 [M+H]⁺

To a stirring solution of intermediate 20 (80 mg, 0.31 mmol) and Compd-1 (50 mg, 0.31 mmol) was added HATU (1.2 eq) and DIPEA (2 ml). The resulted mixture was stirred at room temperature for 1 hour. The reaction mixture washed with water and extracted with EtOAc, the organic phase separated and dried with 27, concentrated in vacuum and pureed by Prep HPLC to afford 20 mg of desired product 32-1 yield: 16%. LC-MS: m/z=406.1 [M+H]⁺ at RT 1.59 (100.00% purity). ¹H NMR (400 MHz, MeOD) δ 7.49-7.18 (m, 9H), 4.77-4.00 (m, 5H), 3.16 (dt, J=8.9, 6.4 Hz, 2H), 2.81-2.67 (m, 1H), 2.35 (dd, J=13.6, 9.1 Hz, 1H), 1.77-1.46 (m, 2H), 1.26-1.07 (m, 3H), 1.00-0.83 (m, 3H).

### Example 28

### 1-cyclobutyl-N-(4-fluorobenzyl)ethanamine (1)

To a stirring mixture of SM1 (300 mg, 3.057 mmol), 4-fluorophenyl)methanamine (383 mg, 3.057 mmol) and acetic acid (183 mg, 3.057 mmol) in MeOH (10 mL) was added NaBH₃CN (385 mg, 6.114 mmol) at room temperature. The reaction mixture was stirred at room temperature overnight. After consumption of the starting material (by TLC), the reaction mixture was diluted with water and extracted with EtOAc. Combined organic extracts were washed with sat. NaHCO₃, dried over anhydrous Na₂SO₄ and concentrated under reduced pressure to afford compound 1 (600 mg, 95%) as a colorless oil. LC-MS: m/z=208 [M+H]⁺

To a stirring solution of compound 1 (287 mg, 1.383 mmol) in DMF (10 mL) was added intermediate 20 (300 mg, 1.153 mmol) and DIPEA (446 mg, 3.459 mmol) followed by HATU (876 mg, 2.306 mmol) at room temperature and stirred at room temperature for two days. The reaction mixture was diluted with water and extracted with EtOAc. Combined organic extracts were dried over anhydrous Na2SO4 and concentrated under reduced pressure to obtain crude product, which was purified by prep-HPLC eluting with ACN/H₂O containing 5% TFA to afford 28-1 (300 mg, 58%) as a white solid. LC-MS: m/z=450.2 [M+H] at RT 4.976 (99.862% purity). ¹H NMR (400 MHz, DMSO-d6): δ 8.81 (d, J=18.2 Hz, 1H), 7.54-7.03 (m, 8H), 4.74-3.91 (m, 5H), 3.06 (dd, J=13.5, 6.6 Hz, 2H), 2.58 (dt, J=10.9, 5.4 Hz, 1H), 2.41 (s, 1H), 2.30-2.11 (m, 1H), 1.94 (s, 1H), 1.83-1.36 (m, 5H), 1.09-0.80 (m, 3H).

### Example 29

To a stirring solution of intermediate 31 (120 mg, 0.35 mmol) and intermediate 11 (66.1 mg, 0.35 mmol) was added HATU (1.2 eq) and DIPEA (2 ml). The resulted mixture was stirred at room temperature for 1 hour. The reaction mixture washed with water and extracted with EtOAc, the organic phase separated and dried with Na₂SO₄, concentrated in vacuum and pureed by Prep HPLC to afford 60 mg of desired product 29-1 yield: 37%. LC-MS: m/z=510.1/512.1 [M+H]⁺ at RT 1.77 (100.00% purity). ¹H NMR (400 MHz, DMSO) δ 8.88 (d, J=21.2 Hz, 1H), 7.62-7.53 (m, 1H), 7.50-7.05 (m, 7H), 4.70-4.30 (m, 3H), 4.26-4.00 (m, 2H) 3.03 (d, J=6.8 Hz, 2H), 2.61 (dd, J=12.7, 6.1 Hz, 1H), 2.42 (s, 1H), 2.27 (dd, J=13.7, 7.8 Hz, 1H), 1.93 (s, 1H), 1.72 (d, J=5.6 Hz, 5H), 1.07-0.86 (m, 3H).

### Example 30

To a stirring solution of intermediate 31 (120 mg, 0.35 mmol) and intermediate 34 (68.6 mg, 0.35 mmol) was added HATU (1.2 eq) and DIPEA (2 mL). The resulted mixture was stirred at room temperature for 1 hour. The reaction mixture washed with water and extracted with EtOAc, the organic phase separated and dried with Na₂SO₄, concentrated in vacuum and pureed by Prep HPLC to afford 65 mg of desired product 30-1 yield: 36.1%. LC-MS: m/z=514.1/516.1 [M+H]⁺ at RT 1.71 (100.00% purity). ¹H NMR (400 MHz, DMSO) δ 8.89 (d, J=14.6 Hz, 1H), 7.58 (d, J=7.6 Hz, 1H), 7.44-7.10 (m, 6H), 4.77-4.10 (m, 4H), 3.71 (d, J=9.2 Hz, 1H), 3.11-2.90 (m, 2H), 2.59 (dd, J=13.5, 5.8 Hz, 1H), 2.25 (dd, J=13.3, 9.7 Hz, 1H), 1.25-1.04 (m, 3H), 0.94 (s, 1H), 0.56-0.10 (m, 4H).

### Example 31

To a stirring solution of intermediate-1 (50 mg, 0.15 mmol, can be prepared by deprotection of Intermediate-36) in DMF (5 mL) was added intermediate-11 (28 mg, 0.15 mmol) and DIPEA (23 mg, 0.18 mmol) at room temperature and stirred for 2 mins, then HATU (62 mg, 0.16 mmol) was added and the reaction mixture was stirred at room temperature for 1 hour. The solvent from the reaction was concentrated under reduced pressure to obtain crude product, which was purified by silica gel column chromatography to afford 31-1 (25 mg, 47%) as an off-white solid. LC-MS: m/z=510.1/512.1 [M+H] at RT1.75 (100% purity). ¹H NMR (400 MHz, DMSO-d6) δ 8.84 (d, J=21.9 Hz, 1H), 7.58 (s, 1H), 7.47 (d, J=8.8 Hz, 1H), 7.43-7.31 (m, 2H), 7.27 (t, J=7.5 Hz, 2H), 7.19 (t, J=6.3 Hz, 2H), 4.66-4.56 (m, 1H), 4.51-4.35 (m, 2H), 4.27 (d, J=16.8 Hz, 0.5H), 4.09 (dd, J=16.8, 4.0 Hz, 0.5H), 3.99 (s, 1H), 3.06 (dd, J=13.7, 6.8 Hz, 2H), 2.57 (dd, J=13.0, 6.5 Hz, 1H), 2.42 (s, 1H), 2.30-2.18 (m, 1H), 1.92 (s, 1H), 1.70 (m, 4H), 1.49 (s, 1H), 1.05-0.85 (m, 3H).

### Example 32

To a stirring solution of intermediate-1 (50 mg, 0.15 mmol) in DMF (5 mL) was added intermediate-34 (28 mg, 0.15 mmol) and DIPEA (23 mg, 0.18 mmol) at room temperature and stirred for 2 hours, then HATU (62 mg, 0.16 mmol) was added and the reaction mixture was stirred at room temperature for 1 hour. The solvent from the reaction was concentrated under reduced pressure to obtain crude product, which was purified by silica gel column chromatography to afford 32-1 (30 mg, 55%) as an off-white solid. LC-MS: m/z=514.1/516.1 [M+H]⁺ at RT 1.69 (100% purity). ¹H NMR (400 MHz, DMSO) δ 8.84 (d, J=14.6 Hz, 1H), 7.90-7.35 (m, 3H), 7.37-6.96 (m, 4H), 4.72 (s, 1H), 4.60 (s, 1H), 4.51-4.07 (m, 2H), 3.70 (s, 1H), 3.04 (d, J=5.6 Hz, 2H), 2.53 (s, 1H), 2.23 (s, 1H), 1.24-1.00 (m, 3H), 0.93 (s, 1H), 0.47 (s, 1H), 0.36-0.07 (m, 3H).

### Example 33

To a stirring solution of 28-1 (40 mg, 0.1 mmol) in MeOH (4 ml) was added NaOH (36 mg, 1 mmol) followed by H₂O₂ (2 mL), and the mixture was stirred at room temperature for 3 hours then extracted by DCM, Combined organic extracts were dried over anhydrous Na₂SO₄ and concentrated under reduced pressure to obtain pure product.

33-1 as white solid (15 mg, 34%). LC-MS: m/z=461.1 [M+H]⁺ at RT 1.44 (100% purity). ¹H NMR (400 MHz, DMSO d6) δ 8.83 (d, J=17.5 Hz, 1H), 8.05-7.69 (m, 3H), 7.52-7.10 (m, 5H), 4.72-4.60 (m, 2H), 4.35 (ddd, J=27.4, 15.8, 7.2 Hz, 1H), 4.20 (d, J=3.6 Hz, 1H), 3.73 (s, 1H), 3.17-2.97 (m, 2H), 2.65-2.54 (m, 1H), 2.28 (m, 1H), 1.30-1.04 (m, 3H), 0.91 (d, J=33.0 Hz, 1H), 0.47 (s, 1H), 0.42-0.07 (m, 3H).

### Example 34

### 1-oxo-2,3-dihydro-1H-indene-4-carbonitrile (1)

To a solution of 4-bromo-2,3-dihydroinden-1-one SM1 (1 g, 4.74 mol) in DMF (5 mL) were added Zn(CN)₂ (0.55 g, 4.74 mmol) and Pd(PPh₃)₄ (0.14 g, 0.12 mmol), the reaction mixture was under microwave irradiation for 1 hour at 165° C. The solvent was removed under reduced pressure. The crude was purified by flash chromatography on silica gel using a mixture of (PE/EtOAc 5:1) as eluent to afford compound 1 (0.45 g, 60%) as an solid. LC-MS: m/z=158 [M+H]⁺

### 2,5-dioxo-2′,3′-dihydrospiro[imidazolidine-4,1′-indene]-4′-carbonitrile (2)

A mixture of 3-hydrox1-oxo-2,3-dihydro-¹H-indene-4-carbonitrile compound 1 (1.85 g, 11.7 mmol), (NH₄)₂CO₃ (14.7 g, 93.6 mmol) and KCN (4 g, 58.5 mmol) in EtOH/H₂O (30 mL/30 mL) was heated at 100° C. for 15 h in a sealed vial. The reaction mixture was extracted with EtOAc. Combined organic extracts were dried over anhydrous Na₂SO₄ and concentrated under reduced pressure to obtain crude product, which was purified by silica gel column chromatography using EtOAc to afford compound 2 (0.95 g, 36%) as an off-white solid. LC-MS: m/z=228 [M+H]⁺

### tert-butyl 2-(4′-cyano-2,5-dioxo-2′,3′-dihydrospiro[imidazolidine-4,1′-indene]-1-yl)acetate (3)

A mixture of compound 2 (0.95 g, 4.18 mmol), tert-butyl 2-bromoacetate (0.9 g, 4.6 mmol) and K₂CO₃ (0.87 g, 6.27 mmol) was stirred at room temperature for 1 hour. The reaction mixture was diluted with water and extracted with EtOAc. Combined organic extracts were dried over anhydrous Na₂SO₄ and concentrated under reduced pressure. The residue was purified by silica gel column chromatography to afford compound 3 (0.78 g, 56%) as an off-white solid. LC-MS: m/z=342 [M+H]⁺

### 2-(4′-cyano-2,5-dioxo-2′,3′-dihydrospiro[imidazolidine-4,1′-indene]-1-yl)acetic acid (4)

To a solution of compound 3 (0.37 g, 1.08 mmol) in DCM (5 mL) was added TFA (2.5 mL). The mixture was stirred at room temperature for 1.5 h. The solvent was concentrated under reduced pressure. The residue was used to the next step without purification. LC-MS: m/z=286 [M+H]⁺

To a stirring solution of compound 4 160 mg, 0.56 mmol), intermediate-14 (118 mg, 0.67 mmol) and DIPEA (144 mg, 1.12 mmol) in DMF (2 mL) was added HATU (320 mg, 0.84 mmol). The mixture was stirred at room temperature for 1 hour. The mixture was diluted with water and extracted with EtOAc. Combined organic extracts were washed with brine and dried over anhydrous Na2SO4, and concentrated under reduced pressure. The residue was purified by Prep-HPLC to afford compound 34-1 (120 mg, 50%) as an off-white solid. LC-MS: m/z=443 [M+H]⁺ at RT 1.61 (96.4% purity). ¹H NMR (400 MHz, DMSO-d6): δ 8.91 (d, J=17.7 Hz, 1H), 7.84 (d, J=7.6 Hz, 1H), 7.70-7.62 (m, 1H), 7.51 (dd, J=15.8, 7.9 Hz, 1H), 7.44-7.16 (m, 5H), 4.75 (s, 1H), 4.63 (d, J=6.6 Hz, 1H), 4.52-4.10 (m, 2H), 3.73 (d, J=9.2 Hz, 1H), 3.37 (d, J=6.9 Hz, 1H), 3.30-3.12 (m, 2H), 2.65 (dd, J=14.0, 6.0 Hz, 1H), 2.38-2.24 (m, 1H), 1.25-1.05 (m, 3H), 0.94 (s, 1H), 0.58-0.08 (m, 4H).

### Example 35

To a stirring solution of 39-1 (40 mg, 0.09 mmol) in MeOH (2.5 mL) was added NaOH (18 mg, 0.45 mmol) followed by H₂O₂ (1 mL). The reaction mixture was stirred at room temperature overnight. The reaction mixture was diluted with water and extracted with DCM. Combined organic extracts were washed with brine and dried over anhydrous Na₂SO₄ and concentrated under reduced pressure. Compound 35-1 (40 mg, 90%) as an off-white solid was afforded. LC-MS: m/z=461 [M+H]⁺ at RT 1.44 (96.4% purity). ¹H NMR (400 MHz, DMSO-d6): δ 8.83 (d, J=17.1 Hz, 1H), 7.85 (s, 1H), 7.64 (d, J=7.5 Hz, 1H), 7.52-7.13 (m, 7H), 4.75 (s, 1H), 4.63 (d, J=5.5 Hz, 1H), 4.51-4.13 (m, 2H), 3.73 (dd, J=16.2, 7.0 Hz, 1H), 3.44-3.36 (m, 1H), 3.32-3.16 (m, 2H), 2.55 (d, J=5.2 Hz, 1H), 2.27-2.12 (m, 1H), 1.25-1.06 (m, 3H), 0.94 (s, 1H), 0.57-0.07 (m, 4H).

### Example 36

### 1-cyclopropyl-N-(naphthalen-2-ylmethyl)ethanamine (1)

Naphthalen-2-ylmethanamine SM1 (0.3 g, 1.91 mmol), 1-cyclopropylethanone SM2 (0.16 g, 1.91 mmol) and AcOH (0.12 g, 1.91 mmol) were combined in DCM (5 mL) under nitrogen. After stirring for 30 min, sodium triacetoborohydride (0.61 g, 2.87 mmol) was added and the mixture was allowed to stir overnight. The reaction was quenched with NaHCO₃ (a.q), partitioned between EtOAc and water, separated, dried with Na₂SO₄ and concentrated under reduced pressure to obtain crude product, which was purified by silica gel column chromatography eluted with PE/EtOAc to afford compound 1 (0.24 g, 56%) as an oil. LC-MS: m/z=226 [M+H]⁺

To a stirring mixture of intermediate-20 (100 mg, 0.38 mmol), compound 1 (87 mg, 0.38 mmol) and DIPEA (74 mg, 0.57 mmol) in DMF (3 mL) was added HATU (173 mg, 0.456 mmol). The reaction mixture was stirred for ¹H at room temperature. The reaction mixture was diluted with water and extracted with EtOAc. Combined organic extracts were dried over anhydrous Na₂SO₄ and concentrated under reduced pressure. The residue was purified by Prep-HPLC to give compound 36-1 (40 mg, 23%) as an off-white solid. LC-MS: m/z=468 [M+H]⁺ at RT 1.69 (97.8% purity). ¹H NMR (400 MHz, DMSO-d6): δ 8.80 (d, J=23.0 Hz, 1H), 7.98-7.72 (m, 4H), 7.57-7.44 (m, 2H), 7.36-7.21 (m, 4H), 4.92 (s, 1H), 4.81 (d, J=7.0 Hz, 1H), 4.57-4.16 (m, 2H), 3.82 (d, J=9.2 Hz, 1H), 3.44 (s, 1H), 3.05 (dd, J=15.0, 7.4 Hz, 2H), 2.56 (dd, J=12.3, 6.4 Hz, 1H), 2.30-2.13 (m, 1H), 1.30-1.08 (m, 3H), 0.96 (d, J=31.1 Hz, 1H), 0.59-0.10 (m, 4H).

### Example 37

### (5-bromofuran-2-yl)methanol (1)

To a stirring solution of methyl 5-bromofuran-2-carboxylate SM1 (200 mg, 0.98 mmol) in THF (10 mL), was added LiBH₄ (56 mg, 3.9 mmol) at room temperature and stirred at room temperature for overnight. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 20% EA/PE to afford compound 1 (100 mg, 58%). TLC: 20% EA/PE (R_{f}: 0.5). LC-MS: m/z=177.0/179.0 [M+H]⁺

### 2-bromo-5-(bromomethyl)furan (2)

To a solution of (5-bromofuran-2-yl)methanol compound 1 (100 mg, 0.57 mmol), in ether (10 mL) was added PBr₃ (182 mg, 0.68 mmol) at room temperature and stirred at room temperature for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 20% EA/PE to afford compound 2 (100 mg, 73.5%). TLC: 20% EA/PE (R_{f}: 0.6)

### (5-bromofuran-2-yl)methanamine (4)

To a stirring solution of 2-bromo-5-(bromomethyl)furan compound 2 (200 mg, 0.84 mmol) in CHCl₃ (10 mL) was added hexamethylenetetramine compound 3 (117.6 mg, 0.84 mmol) at room temperature and stirred at reflux for 1 hour. the reaction mixture concentrated under reduced pressure, redissolved in MeOH (10 mL) and HCl(1 mL) at room temperature and stirred at reflux for 1 hour. the reaction mixture was diluted with aq Na₂CO₃ and extracted with EtOAc. Combined organic extracts were dried over anhydrous Na₂SO₄ and concentrated under reduced pressure to obtain crude product, which was purified by silica gel column chromatography eluting with 20% EA/PE to afford compound 4 (100 mg, 68%). TLC: 30% EtOAc/Hexane (R_{f}: 0.3). LC-MS: m/z=176.0/178.0 [M+H]⁺

### N-((5-bromofuran-2-yl)methyl)-1-cyclopropylethanamine (6)

To a stirring solution of (5-bromofuran-2-yl)methanamine compound 4 (100 mg, 0.57 mmol) in MeOH (10 mL) was added titanium(IV) isopropoxide (244 mg, 0.86 mmol) at room temperature and stirred at room temperature for 2 hours, NaBH₄ (43.3 mg, 1.14 mmol) was added at room temperature and stirred at room temperature for overnight. The reaction mixture was diluted with water and extracted with EA. Combined organic extracts were dried over anhydrous Na₂SO₄ and concentrated under reduced pressure to obtain crude product, which was purified by silica gel column chromatography to afford compound 6 (50 mg, 36%) as an off-white solid. TLC: 30% EtOAc/Hexane (R_{f}: 0.5). LC-MS: m/z=244.0/246.0 [M+H]⁺

To a stirring solution of N-((5-bromofuran-2-yl)methyl)-1-cyclopropylethanamine compound 6 (50 mg, 0.2 mmol), in DMF (5 mL) was added 2-(2,5-dioxo-2′,3′-dihydrospiro[imidazolidine-4,1′-indene]-1-yl)acetic acid intermediate 20 (53.5 mg, 0.2 mmol), followed by DIEA (51.6 mg, 0.4 mmol) and HATU (114 mg, 0.3 mmol), After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 30% EA/PE to afford 37-1 (20 mg, 20%). LC-MS: m/z=486.0/488.0 [M+H]⁺ at RT 1.65 (100% purity). ¹H NMR (300 MHz, DMSO) δ 8.79 (d, J=7.0 Hz, 1H), 7.47-7.17 (m, 4H), 6.67-6.34 (m, 2H), 6.24 (d, J=3.1 Hz, 1H), 4.81-4.12 (m, 4H), 3.59 (d, J=3.3 Hz, 1H), 3.04 (t, J=7.1 Hz, 2H), 2.63-2.51 (m, 1H), 2.28-2.10 (m, 1H), 1.32-0.93 (m, 5H), 0.67-−0.01 (m, 4H).

### Example 38

### (S)-methyl 1-isocyanato-2,3-dihydro-1H-indene-1-carboxylate (1)

Intermediate 2 (45 mg, 0.24 mmol) was dissolved in 10 mL DCM, triethylamine (25 mg, 0.24 mmol) and triphosgene (24 mg, 0.08 mmol) were added at 0° C. The mixture was stirred overnight at room temperature and the solvent was removed in vacuo. The residue was dissolved in ethyl acetate and the precipitate was filtered off. After evaporating, the crude product was used directly in next step.

Compd-1 (43 mg, 0.2 mmol) and intermediate 6 (49 mg, 0.2 mmol) were dissolved in 10 mL DCM and stirred at room temperature for 3 hours and the solvent was removed under reduced pressure. The residue was dissolved in 5 mL of THF and the solution was added dropwise a solution of LiOH.H₂O (10 mg) in 1 mL water, After stirring for 1 hour, the reaction mixture was concentrated in vacuo, purified by column using 50% EA in PE. 30 mg of desired product 38-1 obtained yield: 35%. LC-MS: m/z=432.1 [M+H]⁺ at RT 1.66 (95.84% purity). ¹H NMR (400 MHz, DMSO) δ 8.80 (d, J=21.1 Hz, 1H), 7.45-7.12 (m, 8H), 4.66-4.37 (m, 3H), 4.28 (d, J=16.6 Hz, 0.5H), 4.10 (d, J=16.6 Hz, 0.5H), 4.05-3.95 (m, 1H), 3.05 (dd, J=13.7, 6.9 Hz, 2H), 2.63-2.53 (m, 1H), 2.42 (s, 1H), 2.30-2.15 (m, 1H), 1.92 (s, 1H), 1.79-1.46 (m, 5H), 0.93 (dt, J=20.1, 9.9 Hz, 3H).

### Example 39

### (S)-methyl 1-isocyanato-2,3-dihydro-1H-indene-1-carboxylate (1)

Intermediate 2 (45 mg, 0.24 mmol) was dissolved in 10 mL DCM, triethylamine (25 mg, 0.24 mmol) and triphosgene (24 mg, 0.08 mmol) were added at 0° C. The mixture was stirred overnight at room temperature and the solvent was removed in vacuo. The residue was dissolved in ethyl acetate and the precipitate was filtered off. After evaporating, the crude product was used directly in next step.

Compd-1 (43 mg, 0.2 mmol) and intermediate 7 (49 mg, 0.2 mmol) were dissolved in 10 mL DCM and stirred at room temperature for 3 hours and the solvent was removed under reduced pressure. The residue was dissolved in 5 mL of THF and the solution was added dropwise a solution of LiOH.H₂O (10 mg) in 1 mL water, After stirring for 1 hour, the reaction mixture was concentrated in vacuo, purified by column using 50% EA in PE. 36 mg of desired product 39-1 obtained yield: 42%. LC-MS: m/z=432.2 [M+H]⁺ at RT 1.66 (95.59% purity). ¹H NMR (400 MHz, DMSO) δ 8.80 (d, J=20.6 Hz, 1H), 7.50-7.09 (m, 8H), 4.50 (ddt, J=23.6, 15.9, 6.8 Hz, 3H), 4.28 (d, J=16.6 Hz, 0.5H), 4.10 (d, J=16.6 Hz, 0.5H), 4.00 (dd, J=10.2, 6.5 Hz, 1H), 3.05 (dd, J=14.1, 7.0 Hz, 2H), 2.57 (dd, J=12.4, 6.4 Hz, 1H), 2.42 (s, 1H), 2.23 (td, J=13.8, 6.7 Hz, 1H), 1.93 (s, 1H), 1.80-1.50 (m, 5H), 1.05-0.86 (m, 3H).

### Example 40

### 1-(thiophen-3-yl)-3-(trimethylsilyl)prop-2-yn-1-ol (1)

To a stirring solution of ethynyltrimethylsilane (3.56 g, 36.3 mmol), in dry THF (100 mL) under Ar was added n-BuLi (2.5M, 12.1 mL, 30.3 mmol) with temperature below 0° C. The resultant mixture was stirred at 0° C. for 1 hour then treated with thiophene-3-carbaldehyde SM1 (3 g, 27 mmol) with temperature below 0° C. The mixture was stirred at 0° C. for 1 hour after addition completed. The mixture was then quenched with H₂O and extracted with EtOAc. Combined organic extracts were dried over anhydrous Na₂SO₄ and concentrated and purified by silica gel column chromatography eluting with 12% EtOAc/Hexane to afford compound 1 (5.6 g, 99%). TLC: 20% EtOAc/Hexane (R_{f}: 0.5)

### 4H-cyclopenta[b]thiophen-6 (5H)-one (2)

To a stirring solution of compound 1 (1 g, 4.8 mmol) in dry toluene (80 mL) was added [Rh(cod)₂](BF₄) (0.39 g, 0.96 mmol), tri-p-tolylphosphine(1.17 g, 3.84 g), 2,2,6,6-tetramethylpiperidine (0.68 g, 4.8 mmol) at room temperature. The reaction mixture was stirred at reflux for 16 h then concentrated and purified by silica gel column chromatography eluting with 12% EtOAc/Hexane to afford compound 2 (0.28 g, 42%). TLC: 20% EtOAc/Hexane (R_{f}: 0.3)

### 4,5-dihydrospiro[cyclopenta[b]thiophene-6,4′-imidazolidine]-2′,5′-dione (3)

To a stirring solution of compound 2 (300 mg, 2.32 mmol) in water (10 mL) and EtOH (10 ml) was added (NH₄)₂CO₃ (2.2 g, 23 mmol) and KCN (1.5 g, 23 mmol) at room temperature. The reaction mixture was heated at 120° C. in a sealed tube overnight. After being cooled to room temperature and concentrated, the residue was purified by silica gel column chromatography eluting with 3% MeOH/DCM to afford compound 3 (170 mg, 35%). LC-MS: m/z=209.1 [M+H]⁺

To a stirring solution of compound 3 (50 mg, 0.24 mmol) in DMF (1 mL) was added K₂CO₃ (66 mg, 0.48 mmol) at room temperature. To this added compound intermediate 13 (71 mg, 0.24 mmol) at room temperature and the reaction mixture was stirred at room temperature for 0.5 h. The mixture was diluted with brine, extracted with EtOAc, the organic phase was washed with brine, dried over anhydrous Na₂SO₄ and concentrated and purified by silica gel column chromatography eluting with 25% EtOAc/Hexane to afford compound 40-1 (50 mg, 49%) as white solid. TLC: 50% EtOAc/Hexane (Rf: 0.4). LC-MS: m/z=424 [M+H]⁺ (98% purity). ¹H NMR (400 MHz, DMSO-d6) δ8.74 (d, J=12.0 Hz, 1H), 7.41 (dd, J=20.7, 5.3 Hz, 3H), 7.31-7.16 (m, 3H), 6.83 (t, J=5.0 Hz, 1H), 4.73 (s, 1H), 4.60 (s, 1H), 4.41-4.18 (m, 2H), 3.06-2.85 (m, 3H), 2.55 (s, 1H), 1.13-0.94 (m, 4H), 0.55-0.09 (m, 5H).

### Example 41

### 1-(thiophen-2-yl)-3-(trimethylsilyl)prop-2-yn-1-ol (1)

To a stirring solution of ethynyltrimethylsilane (3.56 g, 36.3 mmol), in dry THF (100 mL) under Ar was added n-BuLi (2.5M, 12.1 mL, 30.3 mmol) with temperature below 0° C. The resultant mixture was stirred at 0° C. for 1 hour then treated with thiophene-2-carbaldehyde SM1 (3 g, 27 mmol) with temperature below 0° C. The mixture was stirred at 0° C. for 1 hour after addition completed. The mixture was then quenched with H₂O and extracted with EtOAc. Combined organic extracts were dried over anhydrous Na₂SO₄ and concentrated and purified by silica gel column chromatography eluting with 12% EtOAc/Hexane to afford compound 1 (5.6 g, 99%). TLC: 20% EtOAc/Hexane (R_{f}: 0.5)

### 4H-cyclopenta[b]thiophen-6(5H)-one (2)

To a stirring solution of compound 1 (1 g, 4.8 mmol) in dry toluene (80 mL) was added [Rh(cod)₂](BF₄) (0.39 g, 0.96 mmol), trip-tolylphosphine(1.17 g, 3.84 g), 2,2,6,6-tetramethylpiperidine (0.68 g, 4.8 mmol) at room temperature. The reaction mixture was stirred at reflux for 16 h then concentrated and purified by silica gel column chromatography eluting with 12% EtOAc/Hexane to afford compound 2 (0.25 g, 38%). TLC: 20% EtOAc/Hexane (R_{f}: 0.3)

### 4,5-dihydrospiro[cyclopenta[b]thiophene-6,4′-imidazolidine]-2′,5′-dione (3)

To a stirring solution of compound 2 (300 mg, 2.32 mmol) in water (10 mL) and EtOH (10 mL) was added (NH₄)₂CO₃ (2.2 g, 23 mmol) and KCN (1.5 g, 23 mmol) at room temperature. The reaction mixture was heated at 120° C. in a sealed tube overnight. After being cooled to room temperature and concentrated, the residue was purified by silica gel column chromatography eluting with 3% MeOH/DCM to afford compound 3 (30 mg, 7%). LC-MS: m/z=209.1 [M+H]⁺

To a stirring solution of compound 3 (30 mg, 0.14 mmol) in DMF (1 mL) was added K₂CO₃ (40 mg, 0.28 mmol) at room temperature. To this added compound intermediate-13 (43 mg, 0.14 mmol) at room temperature and the reaction mixture was stirred at room temperature for 0.5 h. The mixture was diluted with brine, extracted with EtOAc, the organic phase was washed with brine, dried over anhydrous Na₂SO₄ and concentrated and purified by silica gel column chromatography eluting with 25% EtOAc/Hexane to afford compound 41-1 (12 mg, 20%) as white solid. TLC: 50% EtOAc/Hexane (Rf: 0.4). LC-MS: m/z=423.9 [M+H]⁺ (98% purity). ¹H NMR (400 MHz, DMSO-d6) δ 8.90 (d, J=12.1 Hz, 1H), 7.61 (d, J=4.9 Hz, 1H), 7.44-7.11 (m, 5H), 6.91 (d, J=4.9 Hz, 1H), 4.72 (s, 1H), 4.60 (s, 1H), 4.46-4.15 (m, 2H), 3.76-3.63 (m, 1H), 3.00-2.75 (m, 3H), 2.55 (d, J=6.4 Hz, 1H), 1.13 (dd, J=25.6, 6.6 Hz, 3H), 0.93 (s, 1H), 0.55-0.10 (m, 4H).

### Example 42

### (1H-indol-5-yl)methanamine (compound 1)

To a stirring suspension of LiAlH₄ (452 mg, 12.0 mmol) in THF (10 mL) was added a solution of 1H-indole-5-carbonitrile SM (994 mg, 7.0 mmol) in THF (8 mL) at 0° C. The mixture was warmed to 45° C. and stirred for 16 h. The reaction mixture was quenched with water (0.5 mL), 15% NaOH(0.5 mL) and then water (1.5 mL). The mixture was filtered and concentrated to obtain the residue, which was diluted with EtOAc (30 mL) and washed with water (10 mL) and then brine (10 mL). The organic extracts were dried over anhydrous Na₂SO₄ and concentrated under reduced pressure to give the residue, which was washed with Et₂O (15 mL) to afford (1H-indol-5-yl)methanamine compound 1 (790 mg, 79%) as light yellow solid. ¹H NMR (300 MHz, DMSO-d6): δ 10.94 (s, 1H), 7.44 (s, 1H), 7.28 (d, J=8.0 Hz, 1H), 7.04 (d, J=8.0 Hz, 1H), 6.34 (s, 1H), 3.74 (s, 2H), 1.71 (s, 2H).

### N-((1H-indol-5-yl)methyl)-1-cyclopropylethanamine (compound 3)

To a stirring solution of (1H-indol-5-yl)methanamine compound 1 (292 mg, 2.0 mmol) and 1-cyclopropylethanone compound 2 (185 mg, 2.2 mmol) in methanol (10 mL) and 1,2-dichloroethane (10 mL) was added NaBH(OAc)₃ (1.3 g, 3.0 mmol) followed by AcOH (5 drops). The mixture was stirred at 45° C. for 1 hour. The reaction mixture was concentrated and diluted with dichloromethane (30 mL) and washed with water (20 mL) and then brine (10 mL). The organic extracts were dried over anhydrous Na₂SO₄ and concentrated under reduced pressure to afford N-((1H-indol-5-yl)methyl)-1-cyclopropylethanamine compound 3 (500 mg, crude) without further purification. LC-MS: m/z=215.2 [M+H]⁺

### N-((1H-indol-5-yl)methyl)-N-(1-cyclopropylethyl)-2-(2,5-dioxo-2′,3′-dihydrospiro[imidazolidine-4,1′-indene]-1-yl)acetamide

To a stirring solution of N-((1H-indol-5-yl)methyl)-1-cyclopropylethanamine compound 3 (220 mg, crude) and 2-(2,5-dioxo-2′,3′-dihydrospiro[imidazolidine-4,1′-indene]-1-yl)acetic acid Intermediate-20 (260 mg, 1.0 mmol) in DMF (10 mL) was added HATU (760 mg, 2.0 mmol) followed by DIPEA (390 mg, 3.0 mmol). The mixture was stirred at room temperature for 1 hour. The reaction mixture was diluted with water (10 mL) and then extracted with EtOAc (20 mL×2). Combined organic extracts were dried over anhydrous Na₂SO₄ and concentrated under reduced pressure to obtain crude product, which was purified by Prep-HPLC to afford N-((1H-indol-5-yl)methyl)-N-(1-cyclopropylethyl)-2-(2,5-dioxo-2′,3′-dihydrospiro[imidazolidine-4,1′-indene]-1-yl)acetamide (40 mg, 9% two steps) as an off-white solid. ¹H NMR (400 MHz, CD₃OD-d4): δ 7.63-7.03 (m, 7H), 6.52-6.35 (m, 1H), 4.84-4.70 (m, 2H), 4.60-4.36 (m, 2H), 3.87-3.71 (m, 1H), 3.24-3.06 (m, 2H), 2.78-2.64 (m, 1H), 2.44-2.25 (m, 1H), 1.32-1.12 (m, 3H), 1.11-0.98 (m, 1H), 0.65-0.50 (m, 1H), 0.31-0.57 (m, 3H). LC-MS: m/z=457.2 [M+H]⁺ at RT 4.36 (100% purity)

To a stirring solution of N-((1H-indol-5-yl)methyl)-1-cyclopropylethanamine compound 3 (220 mg, crude) and 2-(2,5-dioxo-2′,3′-dihydrospiro[imidazolidine-4,1′-indene]-1-yl)acetic acid intermediate-20 (260 mg, 1.0 mmol) in DMF (10 mL) was added HATU (760 mg, 2.0 mmol) followed by DIPEA (390 mg, 3.0 mmol). The mixture was stirred at room temperature for 1 hour. The reaction mixture was diluted with water (10 mL) and then extracted with EtOAc (2×20 mL). Combined organic extracts were dried over anhydrous Na₂SO₄ and concentrated under reduced pressure to obtain crude product, which was purified by Prep-HPLC to afford 42-1 (40 mg, 9% two steps) as an off-white solid. LC-MS: m/z=457.2 [M+H]⁺ at RT 4.36 (100% purity). ¹H NMR (400 MHz, CD3OD-d4): δ 7.63-7.03 (m, 7H), 6.52-6.35 (m, 1H), 4.84-4.70 (m, 2H), 4.60-4.36 (m, 2H), 3.87-3.71 (m, 1H), 3.24-3.06 (m, 2H), 2.78-2.64 (m, 1H), 2.44-2.25 (m, 1H), 1.32-1.12 (m, 3H), 1.11-0.98 (m, 1H), 0.65-0.50 (m, 1H), 0.31-0.57 (m, 3H).

### Example 43

### ethyl 1-hydroxy-2,3-dihydro-1H-indene-1-carbimidate (1)

To a stirring solution of SM1 (2 g, 12.564 mmol) in dry EtOH (10 mL) was bubbled HCl gas at 0-5° C. The resulting mixture was stirred at room temperature overnight. The solvent was removed under reduced pressure and the residue was titurated with ether to give the undissolved substance, which was then dissolved in water, extracted with EtOAc. The aqueous phase was basified with Na₂CO₃ and extracted with EtOAc. The combined organic extracts were dried over Na₂SO₄ and then filtered. The filtrate was concentrated in vacuo to afford the crude compound 1 (510 mg, 20%) as a clear oil. LC-MS: m/z=206 [M+H]⁺

### 2,3-dihydrospiro[indene-1,5′-ozazolidine]-2′,4′-dione (2)

A mixture of compound 1 (205 mg, 1 mmol) and CDI (162 mg, 1 mmol) was heated to 100° C. and stirred for 16 hours. EtOAc was used to dilute the mixture, which was washed with hydrochloric acid (2N), dried over Na₂SO₄ and then filtered. The filtrate was concentrated in vacuo and purified by prep-HPLC eluting with ACN/H₂O containing 5% TFA to afford compound 2 (90 mg, 44%) as an off-white solid. LC-MS: m/z=204 [M+H]⁺

### tert-butyl 2-(2′,4′-dioxo-2,3-dihydrospiro[indene-1,5′-ozazolidine]-3′-yl)acetate (3)

To a stirring solution of compound 2 (90 mg, 0.443 mmol) in DMF (5 mL) was added K₂CO₃ (122 mg, 0.886 mmol) followed by tert-butyl 2-bromoacetate (87 mg, 0.443 mmol) at room temperature and stirred at room temperature overnight. The reaction mixture was diluted with water and extracted with EtOAc. Combined organic extracts were dried over anhydrous Na₂SO₄ and concentrated under reduced pressure to obtain compound 3 (145 mg, 99%) as a yellow oil. LC-MS: m/z=262 [M+H-t-Bu]⁺

### 2-(2′,4′-dioxo-2,3-dihydrospiro[indene-1,5′-ozazolidine]-3′-yl)acetic acid (4)

To a stirring solution of compound 3 (145 mg, 0.457 mmol) in DCM (1 mL) was added TFA (1 mL) at room temperature and stirred at room temperature for 1 hour. After consumption of the starting material (by TLC), the reaction mixture was concentrated under reduced pressure to obtain crude compound 4 (119 mg, 99%) as a yellow oil. LC-MS: m/z=262 [M+H]⁺

To a stirring solution of compound 4 (119 mg, 0.456 mmol) in DMF (5 mL) was added intermediate 14 (96 mg, 0.547 mmol) and DIPEA (176 mg, 1.368 mmol) followed by HATU (347 mg, 0.912 mmol) at room temperature and stirred at room temperature overnight. The reaction mixture was diluted with water and extracted with EtOAc. Combined organic extracts were dried over anhydrous Na₂SO₄ and concentrated under reduced pressure to obtain crude product, which was purified by prep-HPLC eluting with ACN/H₂O containing 5% TFA to afford 43-1 (150 mg, 79%) as a yellow solid. LC-MS: m/z=419.2 [M+H]⁺ at RT 5.269 (100% purity). ¹H NMR (400 MHz, DMSO): δ 7.70-7.10 (m, 9H), 4.86-4.23 (m, 4H), 3.78 (dd, J=16.1, 9.2 Hz, 0.6H), 3.43-3.35 (m, 0.4H), 3.25-2.98 (m, 2H), 2.72-2.53 (m, 2H), 1.27-1.06 (m, 3H), 0.97 (d, J=3.0 Hz, 1H), 0.57-0.08 (m, 4H).

### Example 44

### N-benzyl-2-(2,5-dioxo-2′,3′-dihydrospiro[imidazolidine-4,1′-indene]-1-yl)-N-(pentan-3-yl)acetamide

To a stirring solution of N-benzylpentan-3-amine SM1 (50 mg, 0.28 mmol), in DMF (5 mL) was added 2-(2,5-dioxo-2′,3′-dihydrospiro[imidazolidine-4,1′-indene]-1-yl)acetic acid compound 2 (72.8 mg, 0.28 mmol), followed by DIEA (72.2 mg, 0.56 mmol) and HATU (160 mg, 0.42 mmol), After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 30% EA/PE to afford 44-1 (30 mg, 25.6%). TLC: 30% EA/PE (R_{f}: 0.3). LC-MS: m/z 420 [M+H]⁺. ¹H NMR (300 MHz, DMSO) δ 8.79 (d, J=7.0 Hz, 1H)), 7.47-7.17 (m, 4H), 6.67-6.34 (m, 2H), 6.24 (d, J=3.1 Hz, 1H), 4.81-4.12 (m, 4H), 3.59 (d, J=3.3 Hz, ¹H), 3.04 (t, J=7.1 Hz, 2H), 2.63-2.51 (m, 1H), 2.28-2.10 (m, 1H), 1.32-0.93 (m, 5H), 0.67-−0.01 (m, 4H).

### Example 45

To a stirring solution of intermediate 21 (50 mg, 0.25 mmol) in DMF (1 mL) was added K₂CO₃ (69 mg, 0.5 mmol) followed by intermediate 13 (73.75 mg, 0.25 mmol) at room temperature and stirred at room temperature for 1 hour. The reaction mixture was diluted with water and extracted with EtOAc. Combined organic extracts were dried over anhydrous Na₂SO₄ and concentrated under reduced pressure to obtain crude product, which was purified by Prep-HPLC to afford 45-1 (12 mg, 11%) as a white solid. LC-MS: m/z=448.0 [M+H]⁺. ¹H NMR (400 MHz, MeOD): δ 7.53-7.17 (m, 9H), 5.18 (dd, J=11.3, 5.5 Hz, 1H)), 4.85-4.74 (m, 2H), 4.59-4.38 (m, 2H), 3.91-3.76 (m, 1H)), 3.52 (d, J=3.5 Hz, 3H), 2.73-2.59 (m, 2H), 1.28 (dd, J=33.9, 6.2 Hz, 3H), 0.97 (d, J=8.5 Hz, 1H), 0.66-0.53 (m, 1H)), 0.43-0.23 (m, 3H).

### Example 46

### 2-ethylhexyl 3-(1-oxo-2,3-dihydro-1H-inden-5-ylthio)propanoate (1)

To a stirring solution of SM1 (4.2 g, 20 mmol), Pd₂(dba)₃ (460 mg, 0.5 mmol), Xantphors (580 mg, 1 mmol) in dioxane (100 mL) was added DIPEA (7.8 g 60 mmol) and 2-ethylhexyl 3-mercaptopropanoate (8.74 g, 40 mmol) under Ar. The mixture was heated to reflux for 2 hours. After being cooled to room temperature and concentrated, the residue was purified by silica gel column chromatography eluting with 25% EtOAc/Hexane to afford compound 1 (6.75 g, 98%). LC-MS: m/z=349.1 [M+H]⁺

### 5-mercapto-2,3-dihydro-1H-inden-1-one (2)

To a stirring solution of compound 1 (6.6 g, 18.9 mmol) in dry THF (200 mL) and dry EtOH (20 ml) was added NaOEt (2.58 g, 38 mmol) at room temperature in one portion. The reaction mixture was stirred at room temperature for 1 hour then concentrated and purified by silica gel column chromatography eluting with 40% EtOAc/Hexane to afford compound 2 (2.5 g, 81%). LC-MS: m/z=165.1 [M+H]⁺

### 5-(methylthio)-2,3-dihydroinden-1-one (3)

To a stirring solution of compound 2 (2.34 g, 14.2 mmol) in dry DMF (40 mL) was added K₂CO₃ (3.9 g, 28 mmol) and MeI (4.05 g, 28 mmol) at room temperature. The reaction mixture was stirred at room temperature for 2 hours then diluted with water and extracted with EtOAc. Combined organic extracts were dried over anhydrous Na₂SO₄ and concentrated and purified by silica gel column chromatography eluting with 30% EtOAc/Hexane to afford compound 3 (1.2 g, 44%). LC-MS: m/z=179.1 [M+H]⁺

### 5′-(methylthio)-2′,3′-dihydrospiro[imidazolidine-4,1′-indene]-2,5-dione (4)

To a stirring solution of compound 3 (0.4 g, 2.25 mmol) in water (10 mL) and EtOH (10 mL) was added (NH₄)₂CO₃ (2.16 g, 22.5 mmol) and KCN (0.73 g, 11.2 mmol) at room temperature. The reaction mixture was heated at 100° C. in a sealed tube overnight. After being cooled to room temperature and concentrated, the residue was purified by silica gel column chromatography eluting with 100% EtOAc/Hexane to afford compound 4 (0.3 g, 54%). LC-MS: m/z=249.2 [M+H]⁺

To a stirring solution of compound 4 (60 mg, 0.2 mmol) in DMF (1 mL) was added K2CO3 (60 mg, 0.4 mmol) at room temperature. To this added compound intermediate 13 (50 mg, 0.2 mmol) at room temperature and the reaction mixture was stirred at room temperature for 0.5 h. The mixture was diluted with brine, extracted with EtOAc, the organic phase was washed with brine, dried over anhydrous Na₂SO₄ and concentrated and purified by silica gel column chromatography eluting with 50% EtOAc/Hexane to afford compound 46-1 (0.05 g, 53%) as white solid. TLC: 50% EtOAc/Hexane (Rf: 0.3). LC-MS: m/z=464 [M+H]⁺ (98.19% purity). ¹H NMR (400 MHz, DMSO-d6): δ 8.76 (d, J=12.7 Hz, 1H), 7.51-7.11 (m, 8H), 4.73 (s, 1H), 4.61 (s, 1H), 4.48-4.16 (m, 2H), 3.71 (s, 1H), 3.36 (s, 0.5H), 3.06-2.95 (m, 2H), 2.55 (d, J=6.8 Hz, 0.5H), 2.19 (dd, J=13.7, 7.9 Hz, 2H), 1.21-1.06 (m, 3H), 1.01-0.86 (m, 2H), 0.50-0.10 (m, 5H).

### Example 47

### 7′-chloro-2′,3′-dihydrospiro[imidazolidine-4,1′-indene]-2,5-dione (2)

To a suspension of 7-chloro-2,3-dihydroinden-1-one 1 (0.5 g, 3 mmol) in EtOH (10 mL) and H₂O (10 mL) was added KCN (0.292 g, 4.5 mmol) and (NH₄)₂CO₃ (1.15 g, 12 mmol), the reaction mixture was stirred in steel tube at 90° C. for 16 h. Cooled to room temperature and the reaction mixture was diluted with cold water and extracted with EtOAc. Combined organic extracts were dried over anhydrous Na₂SO₄ and concentrated under reduced pressure to obtain crude product, which was purified by silica gel column chromatography (EtOAc/Hexane 1:1) to afford compound 2 (0.6 g, 85%) as white solid. LC-MS: m/z=236/238 [M+H]⁺ (95% purity) To a mixture of 7′-chloro-2′,3′-dihydrospiro[imidazolidine-4,1′-indene]-2,5-dione 2 (118 mg, 0.5 mmol) and intermediate 15 (155 mg, 0.5 mmol) in DMF (5 mL) was added K₂CO₃ (207 mg, 1.5 mmol) and stirred at room temperature for 1 hour. After consumption of the starting material (by TLC), diluted with water and extracted with EtOAc. Combined organic extracts were dried over anhydrous Na₂SO₄ and concentrated under reduced pressure to obtain crude product, which was purified by PREP-HPLC (MeCN/H₂O 3:2) to afford 47-1 (95 mg, 40%) as white solid. LC-MS: m/z=466.1/468.1 [M+H]⁺ at RT 4.97 (99.67% purity). ¹H NMR (400 MHz, DMSO-d6) δ 8.85 (d, J=20.4 Hz, 1H), 7.62-7.02 (m, 8H), 4.70-4.21 (m, 4H), 4.10 (d, J=48.8 Hz, 1H), 3.20-2.96 (m, 2H), 2.59 (d, J=6.6 Hz, 1H), 2.45-2.30 (m, 2H), 1.93 (s, 1H), 1.69 (d, J=5.7 Hz, 5H), 1.08-0.79 (m, 3H).

### Example 48

### N-benzyl-2-(5′-bromo-2,5-dioxo-2′,3′-dihydrospiro[imidazolidine-4,1′-indene]-1-yl)-N-(1-cyclopropylethyl)acetamide (1)

To a stirring solution of Intermediate-10 (393 mg, 1.4 mmol) and Intermediate-13 (413 mg, 1.4 mmol) in DMF (5 mL) was added K₂CO₃ (386 mg, 2.8 mmol) and stirred at room temperature for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 50% EtOAc/PE to afford compound 1 (0.48 g, 69%) as an off-white solid. LC-MS: m/z=496.1/498.1 [M+H]⁺

### N-benzyl-N-(1-cyclopropylethyl)-2-(5′-formyl-2,5-dioxo-2′,3′-dihydrospiro[imidazolidine-4,1′-indene]-1-yl)acetamide (1)

To a stirring solution of compound 1 (0.48 g, 0.96 mmol) in THF (20 mL) was added 2.5 M n-BuLi (1.2 mL, 2.88 mmol) and stirred for 15 min at −78° C. Then DMF (0.14 g, 1.92 mmol) was added to the reaction mixture and stirred for 30 min at −78° C. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 50% EtOAc/PE to afford compound 2 (0.22 g, 51%). LC-MS: m/z=446.2 [M+H]⁺

To a stirring solution of compound 2 (220 mg, 0.49 mmol) in CH₃OH/THF (10 mL/1 mL, V/V) was added NaBH₄ (37 mg, 0.98 mmol) at 0° C. and stirred at room temperature for 30 min. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by Prep-HPLC to afford compound 48-1 (0.15 g, 68%). LC-MS: m/z=448.2 [M+H]⁺ at RT 3.97 (98.11% purity). ¹H NMR (300 MHz, DMSO-d6) δ 8.76 (d, J=12.9 Hz, 1H), 7.45-7.06 (m, 8H), 4.67 (d, J=36.8 Hz, 2H), 4.51-4.25 (m, 3H), 4.18 (d, J=2.3 Hz, 1H), 3.75-3.68 (s, 0.5H), 3.42-3.31 (m, 0.5H), 3.01 (dd, J=11.4, 6.9 Hz, 2H), 2.61-2.51 (m, 1H), 2.20 (dd, J=13.3, 7.7 Hz, 1H), 1.21-1.04 (m, 3H), 0.97-0.92 (m, 1H), 0.52-0.09 (m, 4H).

### Example 49

### N-benzyl-N-(1-cyclopropylethyl)-2-(5′-((difluoromethoxy)methyl)-2,5-dioxo-2′,3′-dihydrospiro[imidazolidine-4,1′-indene]-1-yl)acetamide

To a stirring solution of N-benzyl-N-(1-cyclopropylethyl)-2-(5′-(hydroxymethyl)-2,5-dioxo-2′,3′-dihydrospiro[imidazolidine-4,1′-indene]-1-yl)acetamide 53-1 (75 mg, 0.17 mmol) in CH₃CN (10 mL) was added 2,2-difluoro-2-(fluorosulfonyl)acetic acid SM (33 mg, 0.187 mmol) followed by CuI (7 mg, 0.037 mmol) at room temperature. The reaction mixture was heated to 80° C. for 8 hours. After consumption of the starting material (by TLC), the reaction mixture was concentrated under reduced pressure to obtain crude product, which was purified by Prep-HPLC to afford compound 49-1 (13 mg, 15%) as an off-white solid. LC-MS: m/z=498.2 [M+H]⁺ at RT 4.92 (93.18% purity)

### Example 50

### N-benzyl-2-(5′-bromo-2,5-dioxo-2′,3′-dihydrospiro[imidazolidine-4,1′-indene]-1-yl)-N-(1-cyclopropylethyl)acetamide (1)

To a stirring solution of compound SM1 (200 mg, 0.711 mmol) in DMF (8 mL) was added K₂CO₃ (196 mg, 1.422 mmol) followed by intermediate 13 (211 mg, 0.711 mmol) at room temperature and stirred at room temperature overnight. The reaction mixture was diluted with water and extracted with EtOAc. Combined organic extracts were dried over anhydrous Na₂SO₄ and concentrated under reduced pressure to obtain compound 1 (128 mg, 99%) as a colorless oil. LC-MS: m/z=496/498 [M+H]⁺

To a stirring solution of compound 1 (222 mg, 0.302 mmol) in toluene (2 mL), H₂O (0.1 mL) was added cyclopropylboronic acid (50 mg, 0.58 mmol), tricyclohexylphosphine (12.5 mg, 0.0447 mmol) and K₃PO₄ (332 mg, 1.565 mmol) followed by Pd(OAc)₂ (5 mg, 0.0224 mmol) at room temperature. The reaction mixture was stirred at 100° C. under N₂ overnight. EtOAc was added and then filtered through a short silica gel column. The filtrate was concentrated under reduced pressure to obtain crude product, which was purified by prep-HPLC eluting with ACN/H₂O containing 5% TFA to afford 50-1 (62 mg, 30%) as a white solid. LC-MS: m/z=458.0 [M+H]⁺ at RT 5.081 (98.033% purity). ¹H NMR (400 MHz, DMSO): δ 8.72 (d, J=16.6 Hz, 1H), 7.63-6.86 (m, 8H), 4.90-4.06 (m, 4H), 3.71 (s, 0.6H), 3.38 (s, 0.4H), 3.17-2.83 (m, 2H), 2.27-2.07 (m, 1H), 1.92 (d, J=3.3 Hz, 1H), 1.30-1.04 (m, 3H), 0.94 (d, J=8.3 Hz, 3H), 0.66 (s, 2H), 0.57-0.04 (m, 5H).

### Example 51

### 3′-methyl-2′,3′-dihydrospiro[imidazolidine-4,1′-indene]-2,5-dione (1)

To a stirring solution of SM 1 (0.5 g, 3.42 mmol) in EtOH (7 mL), H₂O (7 mL) was added (NH₄)₂CO₃ (1.642 g, 17.1 mmol) followed by potassium cyanide (445 mg, 6.84 mmol) at room temperature in a sealed tube. The reaction mixture was heated to 95° C. overnight. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 66% PE/EtOAc to afford compound 1 (0.7 g, 95%) as a off-white solid. LC-MS: m/z=217 [M+H]⁺

To a stirring solution of compound 1 (80 mg, 0.37 mmol) in DMF (3 mL) was added intermediate 15 (115 mg, 0.37 mmol) followed by K₂CO₃ (102 mg, 0.74 mmol) at room temperature and stirred at room temperature overnight. EtOAc was added and then filtered. The filtrate was concentrated under reduced pressure to obtain crude product, which was purified by prep-HPLC eluting with ACN/H₂O containing 5% TFA to afford 51-1 (70 mg, 42%) as a white solid. LC-MS: m/z=446.2 [M+H]⁺ at RT 5.161 (98.026% purity). ¹H NMR (400 MHz, DMSO): δ 8.78 (t, J=22.6 Hz, 1H), 7.53-7.03 (m, 9H), 4.72-3.84 (m, 5H), 3.50-3.39 (m, 1H), 2.88-2.67 (m, 0.6H), 2.43 (d, J=7.7 Hz, 1.4H), 2.17-2.05 (m, 0.5H), 1.97-1.41 (m, 6.5H), 1.41-1.17 (m, 3H), 1.03-0.78 (m, 3H).

### Example 52

### 5-(difluoromethoxy)-2,3-dihydro-1H-inden-1-one (2)

To a stirring solution of 5-hydroxy-2,3-dihydro-1H-inden-1-one SM1 (1 g, 6.8 mmol) in DMF (30 mL) was added ClCF₂COONa (2.6 g, 16.9 mmol) and K₂CO₃ (2.82 g, 20.4 mmol), the mixture was stirred at room temperature overnight. After consumption of the starting material (by TLC), the reaction mixture was diluted with brine and extracted with EtOAc. Combined organic extracts were dried over anhydrous Na₂SO₄ and concentrated under reduced pressure to obtain crude product, which was purified by silica gel column chromatography to afford compound 2 (0.76 g, 56%) as an off-white solid. TLC: 50% EA/PE. LC-MS: m/z=199 [M+H]⁺

### 5′-(difluoromethoxy)-2′,3′-dihydrospiro[imidazolidine-4,1′-indene]-2,5-dione (3)

A solution of SM2 (076 g, 3.8 mmol), KCN (0.37 g, 5.7 mmol), (NH₄)₂CO₃ (2.7 g, 28.5 mmol) dissolved with EtOH (10 mL) and H₂O (10 mL) in sealed tube was stirred at 75 degree overnight. After consumption of the starting material (by TLC and LC-MS), the solvent from reaction mixture was removed under reduced pressure, the residue was diluted with brine and extracted with DCM(5% in MeOH). Combined organic extracts were dried over anhydrous Na₂SO₄ and concentrated under reduced pressure to obtain crude product, which was purified by silica gel column chromatography to afford compound 3 (0.52 g, 51%) as an off-white solid. TLC: 5% MeOH/DCM. LC-MS: m/z=269 [M+H]⁺

### N-benzyl-N-(1-cyclobutylethyl)-2-(5′-(difluoromethoxy)-2,5-dioxo-2′,3′-dihydrospiro[imidazolidine-4,1′-indene]-1-yl)acetamide

A solution of SM3 (0.1 g, 0.37 mmol), SM4 (0.114 g, 0.37 mmol) and K₂CO₃ (0.102 g, 0.74 mmol) in DMF (10 mL) was stirred for 2 hours. After consumption of the starting material (by TLC), the reaction mixture was diluted with sat NaCl aqueous and extracted with EtOAc. Combined organic extracts were dried over anhydrous Na₂SO₄ and concentrated under reduced pressure to obtain crude product, which was purified by Prep-HPLC [H₂O-CAN(0.05% TFA)] to afford compound 52-1 (0.085 g, 46%) as a white liquid. TLC: 50% PE/EA. ¹H NMR (300 MHz, DMSO-d₆): δ 8.82 (d, J=16.2 Hz, 1H), 7.59-6.92 (m, 8H), 6.85-6.78 (m, 1H), 4.78-4.05 (m, 5H), 3.20-2.87 (m, 2H), 2.87-2.44 (m, 1H), 2.43-2.33 (m, 1H), 2.32-2.20 (m, 1H), 1.81 (m, 1H), 1.75-1.25 (m, 5H), 0.89 (dd, J=6.8, 2.2 Hz, 3H). LC-MS: m/z=498.0 [M+H]⁺ (95.76% purity, 214 nm)

### Example 53

### 4′-methyl-2′,3′-dihydrospiro[imidazolidine-4,1′-indene]-2,5-dione (2)

A solution of 4-methyl-2,3-dihydro-1H-inden-1-one SM1 (1 g, 6.85 mmol), KCN (0.67 g, 10.3 mmol), (NH₄)₂CO₃ (4.9 g, 51 mmol) dissolved with EtOH (30 mL) and H₂O (30 mL) in sealed tube was stirred at 75° C. overnight. After consumption of the starting material (by TLC and LC-MS), the solvent from reaction mixture was removed under reduced pressure, the residue was diluted with brine and extracted with DCM(5% in MeOH). Combined organic extracts were dried over anhydrous Na₂SO₄ and concentrated under reduced pressure to obtain crude product, which was purified by silica gel column chromatography to afford compound 2 (0.8 g, 54%) as an off-white solid. TLC: 10% MeOH/DCM

### N-benzyl-N-(1-cyclopropylethyl)-2-(4′-methyl-2,5-dioxo-2′,3′-dihydrospiro[imidazolidine-4,1′-indene]-1-yl)acetamide

A solution of SM2 (0.216 g, 1 mmol), SM3 (0.295 g, 1 mmol) and K₂CO₃ (0.276 g, 2 mmol) in DMF (10 mL) was stirred for 2 hours. After consumption of the starting material (by TLC), the reaction mixture was diluted with sat NaCl aqueous and extracted with EtOAc. Combined organic extracts were dried over anhydrous Na₂SO₄ and concentrated under reduced pressure to obtain crude product, which was purified by Prep-HPLC [H₂O-CAN(0.05% TFA)] to afford compound 53-1 (0.13 g, 30%) as a white liquid.

¹H NMR (400 MHz, DMSO-d₆): δ 8.78 (d, J=16.5 Hz, 1H), 7.41-7.13 (m, 8H), 4.20-4.75 (m, 4H), 3.77-3.10 (m, 1H), 2.91 (t, J=35.1 Hz, 2H), 2.64-2.40 (m, 1H), 2.36-2.18 (m, 1H), 1.55-1.05 (m, 3H), 0.99 (s, 1H), 0.54-0.21 (m, 4H). LC-MS: m/z=432.0 [M+H]⁺ (96.82% purity, 214 nm)

### Example 54

### N-(2,5-dioxo-2′,3′-dihydrospiro[imidazolidine-4,1′-indene]-5′-yl)acetamide (1)

A mixture of N-(1-oxo-2,3-dihydro-1H-inden-5-yl)acetamide compound intermediate 38 (0.15 g, 0.79 mmol), (NH₄)₂CO₃ (0.62 g, 3.95 mmol) and KCN (0.11 g, 1.59 mmol) in EtOH/H₂O (5 mL/5 mL) was heated at 80° C. for 48 h in a sealed vial. The reaction mixture was extracted with EtOAc. Combined organic extracts were dried over anhydrous Na₂SO₄ and concentrated under reduced pressure to obtain crude product, which was purified by column chromatography on silica gel eluted with EtOAc to give compound 1 (80 mg, 39%) as a white solid. LC-MS: m/z=260 [M+H]⁺

To a stirring solution of compound 1 (75 mg, 0.29 mmol) in DMF (2 mL) was added intermediate-13 (85 mg, 0.29 mmol) followed by K₂CO₃ (60 mg, 0.435 mmol) at room temperature. The reaction mixture was stirred for 1.5 hours at room temperature. The mixture was diluted with water and extracted with EtOAc. Combined organic extracts were dried over anhydrous Na₂SO₄ and concentrated under reduced pressure. The residue was purified by Prep-HPLC to afford compound 54-1 (50 mg, 36%) as an off-white solid. LC-MS: m/z=475 [M+H]⁺ at RT 1.38 (98.96% purity). ¹H NMR (400 MHz, DMSO-d6): δ 9.99 (s, 1H), 8.74 (d, J=16.9 Hz, 1H), 7.64 (d, J=4.6 Hz, 1H), 7.50-7.13 (m, 3H), 4.74 (s, 1H), 4.62 (s, 1H), 4.47-4.11 (m, 1H), 3.80-3.66 (m, 1H), 3.45 (m, 1H), 3.00 (dd, J=13.2, 6.4 Hz, 1H), 2.63-2.54 (m, 1H), 2.27-2.14 (m, 1H), 2.06 (d, J=14.4 Hz, 1H), 1.26-1.04 (m, 2H), 0.94 (s, 1H), 0.52-0.09 (m, 2H).

### Example 55

### diethyl 2-(3-methylbenzyl)malonate (1)

To a stirring solution of 1-(bromomethyl)-3-methylbenzene SM1 (2 g, 10.8 mmol) in DME (20 mL), was added NaH (0.5 g, 13 mmol), followed by diethyl malonate (1.73 g, 10.8 mmol) at 0° C. and stirred at reflux for overnight. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 20% EA/PE to afford compound 1 (1 g, 35%).

TLC: 20% EA/PE (R_{f}: 0.5). LC-MS: m/z=265 [M+H]⁺

### 2-(3-methylbenzyl)malonic acid (2)

To a stirring solution of diethyl 2-(3-methylbenzyl)malonate compound 1 (1 g, 3.8 mmol) in H₂O (50 ml), was added KOH (425.6 mg, 7.6 mmol) at room temperature and stirred at reflux for overnight. After consumption of the starting material (by TLC), the reaction mixture was diluted with aq HCl and extracted with EtOAc. Combined organic extracts were dried over anhydrous Na₂SO₄ and concentrated under reduced pressure to obtain crude compound 2 (600 mg, 76%). TLC: 50% EA/PE (R_{f}: 0.2). LC-MS: m/z=209 [M+H]⁺

### 3-m-tolylpropanoic acid (3)

A solution of 2-(3-methylbenzyl)malonic acid compound 2 (600 mg, 2.88 mmol) in 50% H₂SO₄ (20 mL) was stirred at reflux for overnight. After consumption of the starting material (by TLC), The reaction mixture was diluted with water, and extracted with EA. Combined organic extracts were dried over anhydrous Na₂SO₄ and concentrated under reduced pressure to obtain crude product, which was purified by silica gel column chromatography eluting with 50% EA/PE to afford compound 3 (300 mg, 63.4%). TLC: 50% EtOAc/Hexane (R_{f}: 0.3). LC-MS: m/z=165 [M+H]⁺

### 5-methyl-2,3-dihydroinden-1-one (4)

A solution of 3-m-tolylpropanoic acid compound 3 (300 mg, 1.8 mmol) in PPA (10 mL) was stirred at 75° C. for 1 hour. After consumption of the starting material (by TLC), The reaction mixture was diluted with water, and extracted with EA. Combined organic extracts were dried over anhydrous Na₂SO₄ and concentrated under reduced pressure to obtain crude product, which was purified by silica gel column chromatography eluting with 30% EA/PE to afford compound 4 (100 mg, 37.4%). TLC: 30% EtOAc/Hexane (R_{f}: 0.6). LC-MS: m/z=147 [M+H]⁺

### 5′-methyl-2′,3′-dihydrospiro[imidazolidine-4,1′-indene]-2,5-dione (5)

To a stirring solution 5-methyl-2,3-dihydroinden-1-one compound 4 (100 mg, 0.68 mmol) in EtOH (10 mL), and H₂O (5 mL) was added KCN (65 mg, 1 mmol), followed by (NH₄)₂CO₃ (326 mg, 3.4 mmol) at room temperature and stirred at 110° C. for overnight in sealed tube. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 30% EA/PE to afford compound 5 (100 mg, 68%). TLC: 30% EtOAc/Hexane (R_{f}: 0.4). LC-MS: m/z=217 [M+H]⁺

To a stirring solution of 5′-methyl-2′,3′-dihydrospiro[imidazolidine-4,1′-indene]-2,5-dione compound 5 (50 mg, 0.23 mmol) in DMF (5 mL) was added N-benzyl-2-bromo-N-(1-cyclopropylethyl)acetamide intermediate 13 (67.8 mg, 0.23 mmol), followed by K₂CO₃ (63.5 mg, 0.46 mmol) at room temperature and stirred at room temperature for 2 hours. The reaction mixture was diluted with water and extracted with EA. Combined organic extracts were dried over anhydrous Na₂SO₄ and concentrated under reduced pressure to obtain crude product, which was purified by silica gel column eluting with 30% EA/PE chromatography to afford 55-1 (40 mg, 40%) as an off-white solid. LC-MS: m/z=432 [M+H]⁺ at RT 1.68 (100% purity). ¹H NMR (300 MHz, DMSO) δ 8.71 (s, 1H), 7.54-6.94 (m, 8H), 4.81-4.06 (m, 5H), 3.79-3.59 (m, 1H), 2.97 (s, 2H), 2.29 (s, 3H), 2.22-2.00 (m, 1H), 1.30-0.73 (m, 4H), 0.20 (s, 4H).

### Example 56

### 5′-bromo-2′,3′-dihydrospiro[imidazolidine-4,1′-indene]-2,5-dione(1)

To a stirring solution of SM1 (10 g, 47.4 mmol) in EtOH (50 mL), H₂O (50 mL) was added (NH₄)₂CO₃ (18.2 g, 189.6 mmol) followed by potassium cyanide (6.2 g, 94.6 mmol) at room temperature. The reaction mixture was heated to 80° C. for 12 hours. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 1 (12 g, 91%) as a white solid. LC-MS: m/z=281/283 [M+H]⁺

### N-benzyl-2-(5′-bromo-2,5-dioxo-2′,3′-dihydrospiro[imidazolidine-4,1′-indene]-1-yl)-N—((S)-1-cyclopropylethyl)acetamide (2)

To a stirring solution of compound 1 (1 g, 3.56 mmol) in DMF (5 mL) was added K₂CO₃ (0.97 g, 7.12 mmol) followed SM2 (1.15 g, 3.9 mmol) at room temperature and stirred at room temperature for 2 hours. The reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 2 (1 g, 95%) as a white solid. LC-MS: m/z=496/498 [M+H]⁺

### N-benzyl-2-(5′-cyano-2,5-dioxo-2′,3′-dihydrospiro[imidazolidine-4,1′-indene]-1-yl)-N—((S)-1-cyclopropylethyl)acetamide (3)

A mixture of compound 2 (200 mg, 0.4 mmol), Pd(PPh₃)₄ (46 mg, 0.04 mmol) and Zn(CN)₂ (200 mg, 1.2 mmol) in DMF (5 mL) was stirred at 120° C. for 30 mins. After consumption of the starting material (by TLC), the reaction mixture was concentrated under reduced pressure to obtain crude product. The crude product was purified by chromatography on silica gel PE/EA=10: 1-2:1 to give compound 3 (140 mg, 85%) as a white solid. LC-MS: m/z=444 [M+H]⁺

### 1-(2-(benzyl-((S)-1-cyclopropylethyl)amino)-2-oxoethyl)-2,5-dioxo-2′,3′-dihydrospiro[imidazolidine-4,1′-indene]-5′-carboxamide (4)

To a stirring solution of compound 3 (80 mg, 0.181 mmol) and K₂CO₃ (49 mg, 0.362 mmol) in MeOH (5 mL) was added drop-wise H₂O₂ (1 mL) at 0° C. and stirred at room temperature for 1 hour. The reaction mixture was diluted with water and extracted with DCM. Combined organic extracts were dried over anhydrous Na₂SO₄, filited and concentrated under reduced pressure to give compound 4 (65 mg, 80%) as a white solid. LC-MS: m/z=461 [M+H]⁺

### 1-(2-(benzyl-((S)-1-cyclopropylethyl)amino)-2-oxoethyl)-N-(cyanomethyl)-2,5-dioxo-2′,3′-dihydrospiro[imidazolidine-4,1′-indene]-5′-carboxamide(5)

To a stirring solution of compound 4 (65 mg, 0.1413 mmol) and NaH (11.3 mg, 0.2826 mmol) in dry THF (5 mL) was added drop-wise bromoacetonitrile (17 mg, 0.1413 mmol) at 0° C. and stirred at room temperature for 2 hours. After consumption of the starting material (by TLC), the resulting mixture was added H₂O and extracted with EA, the reaction mixture was concentrated under reduced pressure to obtain crude product. The crude product was Prep-HPLC to afford compound 56-1 (15 mg, 30%) as a white solid. LC-MS: m/z=500 [M+H]⁺. ¹H NMR (400 MHz, DMSO-d6): δ 7.90 (s, 1H), 7.81 (m, 1H), 7.58-7.20 (m, 6H), 4.82-4.69 (m, 1H), 4.55 (ddd, J=11.6, 9.9, 2.7 Hz, 1H), 4.47-4.38 (m, 1H), 4.17 (dd, J=3.4, 1.4 Hz, 1H), 4.13 (dd, J=3.4, 1.4 Hz, 1H), 3.82 (ddd, J=13.6, 8.9, 5.5 Hz, 1H), 3.35-3.26 (m, 1H), 2.77 (ddd, J=14.0, 11.0, 6.9 Hz, 1H), 2.69-2.57 (m, 1H), 1.34-1.29 (m, 1H), 1.22 (dt, J=9.2, 4.6 Hz, 1H), 1.05-0.82 (m, 1H), 0.67-0.47 (m, 1H), 0.51-0.19 (m, 1H).

### Example 57

### 5′-((trimethylsilyl)ethynyl)-2′,3′-dihydrospiro[imidazolidine-4,1′-indene]-2,5-dione (1)

To a stirring solution of intermediate 10 (0.4 g, 1.4 mmol) in DMF/Et₃N (6 ml, v/v=2/1) was added ethynyltrimethylsilane (0.2 g, 2.1 mmol) followed by Pd(PPh₃)₄ (82 mg, 0.07 mmol) and CuI (27 mg, 0.14 mmol) at room temperature. The reaction mixture was refluxing under N₂ overnight. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 1 (0.2 g, 41%). LC-MS: m/z=227.2 [M+H]⁺

To a stirring solution of compound 2 (0.2 g, 0.67 mmol) in DMF (10 mL) was added K₂CO₃ (300 mg, 1.34 mmol) at room temperature and stirred for 15 min. To this added intermediate-13 (200 mg, 0.67 mmol) at room temperature and the reaction mixture was stirred for 1 hour. The solvent from the reaction was removed under reduced pressure. Obtained residue was purified by silica gel column chromatography eluting with 5% DCM/MeOH to afford 57-1 (130 mg, 64%) as an off-white solid. TLC: 5% DCM/MeOH (Rf: 0.15). LC-MS: m/z=442.1 [M+H]⁺ at RT 1.62 (100% purity). ¹H NMR (400 MHz, DMSO) δ 8.82 (d, J=17.6 Hz, 1H), 7.52-7.12 (m, 8H), 4.74 (s, 1H), 4.62 (s, 1H), 4.50-4.23 (m, 1H), 4.20 (d, J=1.6 Hz, 2H), 3.73 (d, J=9.1 Hz, 0.5H), 3.37 (d, J=9.2 Hz, 0.5H), 3.10-2.97 (m, 2H), 2.60-2.54 (m, 1H), 2.21 (d, J=13.2 Hz, 1H), 1.31-1.04 (m, 3H), 0.94 (s, 1H), 0.47 (s, 1H), 0.37 (s, 1H), 0.20 (d, J=18.0 Hz, 2H).

### Example 58

To a stirring solution of 14-1 (0.2 g, 0.53 mmol) in DMF/Et₃N (6 ml, v/v=2/1) was added prop-2-yn-1-ol (0.3 g, 5.3 mmol) followed by Pd(PPh₃)₄ (60 mg, 0.05 mmol) and CuI (15 mg, 0.01 mmol) at room temperature. The reaction mixture was refluxing under N₂ overnight. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford 58-1 (30 mg, 14%) as an off-white solid. TLC: 5% DCM/MeOH (Rf: 0.20). LC-MS: m/z=473.1 [M+H]⁺ at RT 1.52 (100% purity). ¹H NMR (400 MHz, DMSO) δ 8.80 (d, J=17.5 Hz, 1H), 7.48-7.12 (m, 8H), 5.32 (s, 1H), 4.74 (s, 1H), 4.62 (s, 2H), 4.43 (d, J=17.3 Hz, 1H), 4.31 (s, 2H), 4.20 (s, 1H), 3.73 (d, J=8.7 Hz, 1H), 3.03 (d, J=7.0 Hz, 2H), 2.56 (s, 1H), 2.21 (d, J=12.8 Hz, 1H), 1.31-1.03 (m, 3H), 0.95 (s, 1H), 0.47 (s, 1H), 0.36 (s, 1H), 0.22 (s, 2H).

### Example 59

### 6′-methoxy-2′,3′-dihydrospiro[imidazolidine-4,1′-indene]-2,5-dione (1)

To a stirring solution of SM 1 (1.62 g, 10 mmol) in EtOH (20 mL), H₂O (20 mL) was added (NH₄)₂CO₃ (12 g, 120 mmol) followed by potassium cyanide (2 mg, 30 mmol) at room temperature. The reaction mixture was heated to 75° C. overnight. After consumption of the starting material (by TLC), the reaction mixture was diluted with water and extracted with EtOAc. Combined organic extracts were dried over anhydrous Na₂SO₄ and concentrated under reduced pressure to obtain crude product, which was recrystallized from EtOAc to afford compound 1 (1.116 g, 95%) as a off-white solid. LC-MS: m/z=233 [M+H]⁺

To a stirring solution of compound 1 (60 mg, 0.258 mmol) in DMF (2.5 mL) was added intermediate 15 (80 mg, 0.258 mmol) followed by K₂CO₃ (71 mg, 0.516 mmol) at room temperature and stirred at room temperature overnight. EtOAc was added and then filtered. The filtrate was concentrated under reduced pressure to obtain crude product, which was purified by prep-HPLC eluting with ACN/H₂O containing 5% TFA to afford 59-1 (36 mg, 30%) as a white solid. LC-MS: m/z=462.2 [M+H]⁺ at room temperature 4.947 (99.439% purity). ¹H NMR (400 MHz, DMSO): δ 8.80 (d, J=20.5 Hz, 1H), 7.37 (q, J=8.1 Hz, 2H), 7.30-7.12 (m, 4H), 6.96-6.79 (m, 2H), 4.68-4.22 (m, 4H), 4.14-3.93 (m, 1H), 3.70 (dd, J=30.2, 3.9 Hz, 3H), 2.97 (dd, J=13.1, 6.8 Hz, 2H), 2.63-2.54 (m, 1H), 2.41 (s, 1H), 2.33-2.11 (m, 1H), 1.93 (s, 1H), 1.75-1.50 (m, 5H), 1.10-0.80 (m, 3H).

### Example 60

### ethyl 1-isothiocyanato-2,3-dihydro-1H-indene-1-carboxylate (1)

To a stirring solution of ethyl 1-amino-2,3-dihydro-1H-indene-1-carboxylate SM1 (300 mg, 1.5 mmol) in H₂O (10 mL) and DCM (1 ml) was added NaHCO₃ (630 mg, 7.5 mmol) at 0° C. The reaction mixture was then treated with thiophosgene (350 mg, 3 mmol) and stirred at 0° C. for 2 hour then NaBH₄ (2.1 g, 55.2 mmol) was added. The reaction mixture was stirred at room temperature overnight then worked up afford crude compound 1 (250 mg). TLC: 10% EtOAc/Hexane (R_{f}: 0.7)

### 2-amino-N-benzyl-N-(1-cyclobutylethyl)acetamide (2)

To a stirring solution of N-benzyl-2-bromo-N-(1-cyclobutylethyl)acetamide Intermediate-13 (200 mg, 0.66 mmol) in EtOH (3 mL) was added NH₄OH (2 mL) at room temperature. The reaction mixture was heated at 45° C. in a sealed tube for 1 hour then cooled and concentrated to afford crude compound 2 (250 mg). LC-MS: m/z=247.2 [M+H]⁺

### N-benzyl-N-(1-cyclobutylethyl)-2-(5-oxo-2-thioxo-2′,3′-dihydrospiro[imidazolidine-4,1′-indene]-1-yl)acetamide (3)

To a stirring solution of compound 1 (220 mg, 0.9 mmol) and compound 2 (220 mg, 0.9 mmol) in dry THF (10 ml) was added Et₃N (270 mg, 2.7 mmol) at room temperature. The reaction mixture was stirred at room temperature for 1.5 hours then concentrated under reduced pressure to obtain crude product, which was purified by silica gel column chromatography eluting with 15% EtOAc/Hexane to afford compound 3 (160 mg, 40%). LC-MS: m/z=448.2 [M+H]⁺

To a stirring solution of compound 3 (90 mg, 0.2 mmol) in EtOH (2 mL) was added NH₄OH (2 mL) and t-BuOOH (2 mL) at room temperature. The mixture was stirred in a sealed tube at 40° C. for 2 hours then cooled, concentrated and by prep-HPLC to afford 60-1 (40 mg, 38%) as an off-white solid (TFA salt). TLC: 10% MeOH/hexane (Rf: 0.2). LC-MS: m/z=431.1 [M+H]⁺ (98% purity). ¹H NMR (400 MHz, DMSO-d6) δ10.74 (d, J=16.4 Hz, 1H), 9.73-9.32 (m, 2H), 7.47-7.12 (m, 8H), 5.09-4.94 (m, 1H), 4.81-4.68 (d, J=17.7 Hz, 1H), 4.58-4.31 (m, 2H), 3.87 (s, 1H), 2.64-2.55 (m, 1H), 2.46-2.32 (m, 2H), 1.95-1.58 (m, 5H), 1.21-1.55 (m, 3H), 1.05-0.83 (m, 3H).

### Example 61

### N-benzyl-1,1-dicyclopropylmethanamine (1)

To a stirring solution of phenylmethanamine SM1 (2.14 g, 20 mmol) in 1,2-dichloroethane (20 mL) was added dicyclopropylmethanone SM2 (2.2 g, 20 mmol) at room temperature. The reaction mixture was stirred at room temperature for 2 hours. Then NaBH₄CN ((2.5 g, 40 mmol) was added slowly and stirred at room temperature for 16 hours. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 40% EtOAc/PE to afford compound 1 (200 mg, 5%) as an colorless oil. LC-MS: m/z=202.1 [M+H]⁺

To a stirring solution of compound 1 (100 mg, 0.5 mmol) and intermediate 20 (130 mg, 0.5 mmol) in DMF (5 mL) was added DIPEA (260 mg, 1.0 mmol) followed by HATU (760 mg, 1.0 mmol) and stirred at room temperature for 1 hour. The reaction mixture was diluted with water and extracted with DCM. Combined organic extracts were dried over anhydrous Na₂SO₄ and concentrated under reduced pressure to obtain crude product, which was purified by silica gel column chromatography eluting with 60% PE/EtOAc to afford compound 62-1 (74 mg, 33%) as an off-white solid. LC-MS: m/z=444.2 [M+H]⁺ at RT 8.57 (99.76% purity). ¹H NMR ((400 MHz, DMSO-d6) δ 8.78 (d, J=20.3 Hz, 1H), 7.56-7.06 (m, 7H), 4.76 (d, J=44.2 Hz, 2H), 4.20 (d, J=24.2 Hz, 2H), 3.31-3.11 (m, ¹H), 3.03 (dd, J=13.3, 6.5 Hz, 2H), 2.72 (t, J=8.8 Hz, 1H), 2.55 (d, J=6.9 Hz, 1H), 2.35-2.07 (m, 1H), 1.02 (dd, J=7.8, 4.0 Hz, 2H), 0.71-0.02 (m, 6H).

### Example 62

### 6′-chloro-2′,3′-dihydrospiro[imidazolidine-4,1′-indene]-2,5-dione (2)

To a suspension of 6-chloro-2,3-dihydroinden-1-one 1 (0.5 g, 3 mmol) in EtOH (10 mL) and H₂O (10 mL) was added KCN (0.292 g, 4.5 mmol) and (NH₃)₂CO₃ (1.15 g, 12 mmol), the reaction mixture was stirred in steel tube at 100° C. for 6 h. Cooled to room temperature and the reaction mixture was diluted with cold water and extracted with EtOAc. Combined organic extracts were dried over anhydrous Na₂SO₄ and concentrated under reduced pressure to obtain crude product, which was purified by silica gel column chromatography (EtOAc/Hexane 1:1) to afford compound 2 (0.47 g, 67%) as white solid. LC-MS: m/z=236/238 [M+H]⁺ (95% purity)

To a mixture of 6′-chloro-2′,3′-dihydrospiro[imidazolidine-4,1′-indene]-2,5-dione 2 (118 mg, 0.5 mmol) and intermediate 15 (155 mg, 0.5 mmol) in DMF (5 mL) was added K₂CO₃ (207 mg, 1.5 mmol) and stirred at room temperature for 2 hours. After consumption of the starting material (by TLC), diluted with water and extracted with EtOAc. Combined organic extracts were dried over anhydrous Na₂SO₄ and concentrated under reduced pressure to obtain crude product, which was purified by PREP-HPLC (MeCN/H₂O 3:2) to afford 62-1 (100 mg, 43%) as white solid. LC-MS: m/z=466/468 [M+H]⁺ at RT 5.21 (99.18% purity). ¹H NMR (400 MHz, DMSO-d6) δ 8.86 (m, 1H), 7.42-7.32 (m, 5H), 7.50-7.00 (m, 3H), 4.67-4.23 (m, 4H), 4.17-3.92 (m, 1H), 3.03 (d, J=6.9 Hz, 2H), 2.66-2.54 (m, 1H), 2.47-2.19 (m, 2H), 1.93 (s, 1H), 1.70 (dd, J=11.2, 4.7 Hz, 6H), 1.03-0.82 (m, 3H).

### Example 63

To a mixture of 1H-pyrazol-3-ylboronic acid SM (41 mg, 0.36 mmol), N-benzyl-2-(5′-bromo-2,5-dioxo-2′,3′-dihydrospiro[imidazolidine-4,1′-indene]-1-yl)-N-(1-cyclopropylethyl)acetamide 19-1 (60 mg, 0.12 mmol) and NaHCO₃ (50 mg, 0.6 mmol) in dioxane (3 mL)-water (1 mL) was added Pd(dppf)₂Cl₂ (6 mg, 0.006 mmol) under argon atmosphere. The mixture was degassed and sealed, which was stirred at 100° C. for 30 mins under microwave. The reaction mixture was diluted with EtOAc (30 mL) and washed with water (10 mL). The organic extracts was concentrated under reduced pressure to obtain crude product, which was purified by Prep-HPLC to afford 63-1 (38 mg, 66%) as an off-white solid. LC-MS: m/z=484.2 [M+H]⁺ at RT 4.18 (98.56% purity). ¹H NMR (400 MHz, CD₃OD-d4): δ 7.81-7.63 (m, 3H), 7.50-7.38 (m, 6H), 6.74 (s, 1H), 4.85-4.70 (m, 2H), 4.60-4.28 (m, 2H), 3.86-3.82 (m, 1H), 3.25-3.15 (m, 2H), 2.80-2.74 (m, 1H), 2.43-2.35 (m, 1H), 1.36-1.14 (m, 3H), 0.98-0.94 (m, 1H), 0.62-0.54 (m, 1H), 0.43-0.16 (m, 3H).

### Example 64

### N-(4-chlorobenzyl)-N—((S)-1-cyclobutylethyl)-2-methylpropane-2-sulfinamide (1)

To a stirring solution of N—((S)-1-cyclobutylethyl)-2-methylpropane-2-sulfinamide Intermediate-16 (329 mg, 1.5 mmol) in DMF (2 mL) was added NaH (120 mg, 3 mmol) at 0° C. and stirred at 0° C. for 30 min. Then 1-(bromomethyl)-4-chlorobenzene SM (308 mg, 1.5 mmol) was added and stirred at room temperature for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 25% EtOAc/Hexane to afford compound 1 (0.29 g, 56%).

### (S)—N-(4-chlorobenzyl)-1-cyclobutylethanamine hydrochloride (2)

To a stirring solution of compound 1 (0.29 g, 0.88 mmol) in MeOH (10 mL) was added 2N HCl in dioxane (2 mL) and stirred at room temperature for 30 min. The reaction mixture was concentrated under reduced pressure to obtain compound 2 (228 mg, 100%) as a white solid used for next step directly. LC-MS: m/z=223.2/225.2 [M+H]⁺

To a stirring solution of compound 2 (130 mg, 0.5 mmol) and intermediate-9 (130 mg, 0.5 mmol) in DMF (2 mL) was added HATU (285 mg, 0.75 mmol) followed by DIPEA (129 mg, 1 mmol) and stirred at room temperature for 1 hour. The reaction mixture was diluted with water and extracted with EtOAc. Combined organic extracts were dried over anhydrous Na2SO4 and concentrated under reduced pressure to obtain crude product, which was purified by prep-hplc to afford compound 64-1 (70 mg, 30%) as an off-white solid. LC-MS: m/z=466.1 [M+H]⁺ at RT 5.22 (99.55% purity). ¹H NMR (300 MHz, DMSO-d6) δ 8.80 (d, J=13.4 Hz, 1H), 7.58-7.06 (m, 8H), 4.65-4.38 (m, 3H), 4.28-4.23 (d, J=16.7 Hz, 0.5H), 4.02 (dd, J=17.2, 10.0 Hz, 1H), 3.39-3.34 (m, 0.5H), 3.04 (dd, J=11.9, 6.9 Hz, 2H), 2.62-2.51 (m, 1H), 2.39 (s, 1H), 2.28-2.14 (m, 1H), 1.92 (s, 1H), 1.77-1.39 (m, 5H), 0.94 (dd, J=33.0, 6.6 Hz, 3H).

### Example 65

To a stirring solution of 57-1 (60 mg, 0.14 mmol) in DMSO/H₂O (6 ml, v/v=9/1) was added NaN₃ (27 mg, 0.41 mmol) followed by DIPEA (9 mg, 0.07 mmol), 1 M CuSO₄ 5H₂O (0.14 ml, 0.01 mmol) and ascorbate sodium (0.1 mL 0.02 mmol) was added steeply at room temperature. The reaction mixture was refluxing under N2 for 2 hours. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford 65-1 (15 mg, 30%) as an off-white solid. TLC: 5% DCM/MeOH (Rf: 0.20). LC-MS: m/z=485.1 [M+H]⁺ at RT 1.48 (100% purity). ¹H NMR (400 MHz, DMSO) δ 8.81 (d, J=17.2 Hz, 1H), 8.34-8.23 (m, 1H), 7.80 (m, 2H), 7.39 (d, J=7.6 Hz, 4H), 7.24 (t, J=17.7 Hz, 3H), 4.75 (s, 1H), 4.63 (s, 1H), 4.42 (s, 0.5H), 4.32 (s, 0.5H), 4.21 (s, 1H), 3.73 (s, 1H), 3.10 (d, J=7.1 Hz, 2H), 2.65-2.56 (m, 1H), 2.27 (s, 1H), 1.25-1.06 (m, 3H), 0.96 (s, 1H), 0.49 (s, 1H), 0.36 (s, 1H), 0.23 (s, 2H).

### Example 66

To a stirring solution of compound 1 (55 mg, 0.25 mmol) in DMF (1.5 mL) was added 2 (78 mg, 0.25 mmol) followed by K₂CO₃ (52 mg, 0.375 mmol) at room temperature. The reaction mixture was stirred for 1.5 hours at room temperature. The mixture was diluted with water and extracted with EtOAc. Combined organic extracts were dried over anhydrous Na₂SO₄ and concentrated under reduced pressure. The residue was purified by flash chromatography eluted with PE/EtOAc to afford compound 66-1 (21 mg, 19%) as an off-white solid. LC-MS: m/z=448 [M+H]⁺ at RT 1.54 (98.7% purity). ¹H NMR (400 MHz, DMSO-d6): δ 8.84 (d, J=20.6 Hz, 1H), 7.50-7.11 (m, 10H), 5.75 (s, 1H), 5.24 (t, J=7.0 Hz, 1H), 4.64-4.19 (m, 4H), 4.11-3.91 (m, 1H), 2.84 (dt, J=15.1, 7.6 Hz, 1H), 2.40 (s, 1H), 2.13-2.01 (m, 1H), 1.92 (s, 1H), 1.80-1.42 (m, 5H), 1.07-0.83 (m, 3H).

### Example 67

### N-benzyl-N—((S)-1-cyclobutylethyl)-2-methylpropane-2-sulfinamide (1)

To a solution of N—((S)-1-cyclobutylethyl)-2-methylpropane-2-sulfinamide Intermediate-16 (600 mg, 3 mmol) in DMF (6 mL) were added NaH (180 mg, 4.5 mmol) at 0° C., the reaction mixture was stirred at 0° C. for 10 min, then (bromomethyl)benzene SM1 (0.43 mL, 3.6 mmol) was added, the mixture was allowed to warm to room temperature an stirred for 1 hour. The reaction mixture was quenched with water and extracted with EtOAc. Combined organic extracts were dried over anhydrous Na₂SO₄ and concentrated under reduced pressure to obtain crude product, which was purified by silica gel column chromatography using PE/EtOAc (4:1) to afford compound 1 (850 mg, 91%) as an oil. LC-MS: m/z=294 [M+H]⁺

### (S)—N-benzyl-1-cyclobutylethanamine hydrochloride (2)

A solution of compound 1 (800 mg, 2.73 mmol) in dioxane (5 mL) containing HCl (g) was stirred at room temperature for 2 hours. The solvent was removed under reduced pressure to obtain crude product, which was used to the next step without purification. LC-MS: m/z=190 [M+H]⁺

### (S)—N-benzyl-2-bromo-N-(1-cyclobutylethyl)acetamide (3)

To a mixture of compound 2 (0.86 g, 3.82 mmol) and Et₃N (0.85 g, 8.4 mmol) in DCM (10 mL) was added dropwise 2-bromoacetyl bromide (0.85 g, 4.2 mmol) at 0° C. and then the mixture was stirred for 1 hour. The mixture was quenched with NH₄Cl (a.q) and extracted with DCM (10 mL). Combined organic extracts were dried over anhydrous Na₂SO₄ and concentrated under reduced pressure. The residue was purified by silica gel column chromatography using PE/EtOAc (2:1) to afford compound 3 (650 mg, 54%) as an oil which was used in the next step without purification. LC-MS: m/z=310 [M+H]⁺

To a stirring solution of compound 4 (50 mg, 0.23 mmol) in DMF (1.5 mL) was added compound 3 (71 mg, 0.23 mmol) followed by K₂CO₃ (48 mg, 0.34 mmol) at room temperature. The reaction mixture was stirred for 1.5 hours at room temperature. The mixture was diluted with water and extracted with EtOAc. Combined organic extracts were dried over anhydrous Na2SO4 and concentrated under reduced pressure. The residue was purified by Prep-HPLC to afford 67-1 (30 mg, 29%) as an off-white solid. LC-MS: m/z=448 [M+H]⁺ at RT 1.55 (97.8% purity). ¹H NMR (400 MHz, DMSO-d6): δ 8.76 (m, 1H), 7.47-7.15 (m, 9H), 5.56 (d, J=7.1 Hz, 1H), 5.39-5.26 (m, 1H), 4.70-4.27 (m, 5H), 4.20-3.96 (m, 2H), 2.43-2.27 (m, 2H), 1.94 (s, 1H), 1.80-1.50 (m, 6H), 1.07-0.87 (m, 3H).

### Example 68

### N-(4-bromobenzyl)-N—((S)-1-cyclobutylethyl)-2-methylpropane-2-sulfinamide (2)

To a solution of intermediate 16 (0.3 g, 1.6 mmol) in DMF (5 mL) was added NaH (72 mg, 3.2 mmol) at 0° C. under N₂ and stirred at 0° C. for 1 hour. Then 1-bromo-4-(bromomethyl)benzene 1 (0.4 g, 1.6 mmol) was added and stirred at room temperature for 2 hours. Diluted with water and extracted with EtOAc. Combined organic extracts were dried over anhydrous MgSO₄ and concentrated under reduced pressure to obtain crude product, which was purified by silica gel column chromatography (EtOAc/Hexane 1:3) to afford compound 2 (0.3 g, 73%) as oil. ¹H NMR (300 MHz, CDCl₃) δ 7.46 (d, J=8.3 Hz, 2H), 7.21 (d, J=8.3 Hz, 2H), 4.35 (d, J=16.7 Hz, 1H), 3.97 (d, J=16.7 Hz, 1H), 3.04 (dd, J=14.7, 8.1 Hz, 1H), 2.61 (dd, J=16.3, 7.5 Hz, 1H), 2.17-2.02 (m, 1H), 1.96 (d, J=8.0 Hz, 1H), 1.88-1.75 (m, 2H), 1.74-1.52 (m, 2H), 1.21 (d, J=17.0 Hz, 9H), 1.08 (d, J=6.8 Hz, 3H).

### (S)—N-(4-bromobenzyl)-1-cyclobutylethanamine hydrochloride (3)

To a solution of N-(4-bromobenzyl)-N—((S)-1-cyclobutylethyl)-2-methylpropane-2-sulfinamide 2 (0.3 g, 0.8 mmol) in MeOH (2 mL) was added HCl (2N in dioxane, 2 mL) and stirred at room temperature for 1 hour. Concentrated under reduced pressure to obtain crude compound 3 (0.23 g, 94%) as off-white solid. ¹H NMR (300 MHz, DMSO-d₆) δ 9.18 (s, 1H), 9.00 (s, 1H), 7.64 (d, J=8.4 Hz, 2H), 7.55 (d, J=8.4 Hz, 2H), 4.06 (dd, J=11.1, 7.0 Hz, 2H), 3.06 (d, J=6.3 Hz, 1H), 2.59 (d, J=8.3 Hz, 1H), 2.06 (d, J=4.5 Hz, 1H), 2.00-1.61 (m, 5H), 1.17 (d, J=6.6 Hz, 3H).

To a solution of intermediate 9 (40 mg, 0.15 mmol) and (S)—N-(4-bromobenzyl)-1-cyclobutylethanamine hydrochloride 3 (46 mg, 0.15 mmol) in DMF (5 mL) was added DIPEA (47 mg, 0.462 mmol) under N₂ and stirred at room temperature for 15 min. Then HATU (70 mg, 0.18 mmol) was added and stirred at room temperature for 1 hour. After consumption of the starting material (by TLC) diluted with cold water and extracted with EtOAc. Combined organic extracts were dried over anhydrous Na2SO4 and concentrated under reduced pressure to obtain crude product, which was purified by Prep-HPLC (H₂O/MeCN 7:3) to afford compound 68-1 (30 mg, 38%) as white solid. LC-MS: m/z=510.1/512.1 [M+H]⁺ at RT 4.22 (99.22% purity). ¹H NMR (300 MHz, DMSO-d6) δ 8.80 (d, J=13.3 Hz, 1H), 7.56 (d, J=8.4 Hz, 1H), 7.49-7.39 (m, 1H), 7.36-7.20 (m, 5H), 7.15 (d, J=8.4 Hz, 1H), 4.61-4.20 (m, 4H), 4.04 (d, J=16.5 Hz, 1H), 3.04 (dd, J=11.6, 6.9 Hz, 2H), 2.62-2.52 (m, 1H), 2.40 (d, J=8.1 Hz, 1H), 2.21 (dd, J=13.2, 9.2 Hz, 1H), 1.93 (s, 1H), 1.70 (s, 5H), 0.94 (dd, J=33.3, 6.5 Hz, 3H).

### Example 69

### (Z)-ethyl 3-aminobut-2-enoate (1)

To a mixture of ethyl 3-oxobutanoate SM1 (26.0 g, 200 mmol) and silica gel (2 g) was added dropwise 28% ammonia aqueous solution (14.6 g, 240 mmol) at room temperature, and the mixture was stirred at room temperature for 18 hour. The mixture was filtered, and the filtrate was diluted with water and extracted with EtOAc. The extract was washed with brine, dried over anhydrous Na₂SO₄ and concentrated under reduced pressure to obtain crude product, which was purified by silica gel column chromatography eluting with 50% EtOAc/PE to afford compound 1 (21.5 g, 67%) as a colorless oil. LC-MS: m/z=130.1 [M+H]⁺

### (2E,4Z)-5-ethyl 1-methyl 4-(1-aminoethylidene)pent-2-enedioate (2)

A mixture of compound 1 (21.5 g, 133 mmol) and methyl propiolate (11.8 g, 140 mmol) in toluene (140 mL) was stirred under nitrogen atmosphere at reflux for 4 hour. To the mixture was added methyl propiolate (5.64 g, 67.1 mmol), and the mixture was stirred under nitrogen atmosphere at reflux for 12 hour. After cooling, the mixture was concentrated to give crude compound 2 (32 g, 100%) as an orange solid. This product was used for the next step without further purification. LC-MS: m/z=214.1 [M+H]⁺

### ethyl 6-hydroxy-2-methylnicotinate (3)

A solution of crude product compound 2 (32 g, 0.15 mol) in DMF (250 mL) was stirred under nitrogen atmosphere at reflux for 3 days. After evaporation of the solvent, the resulting solid was washed with toluene to give ethyl 6-hydroxy-2-methylnicotinate compound 3 (8.11 g, 34%) as a yellow crystals. LC-MS: m/z=182.1 [M+H]⁺

### ethyl 6-chloro-2-methylnicotinate (4)

A solution of ethyl 6-hydroxy-2-methylnicotinate compound 3 (8.11 g, 44.6 mmol) and phosphorous oxychloride (20.0 g, 130 mmol) was stirred under nitrogen atmosphere at 120° C. for 2 hours. After cooling, the mixture was poured into ice water, basified with 8 M NaOH aqueous solution, and extracted with EtOAc. The extract was washed with brine, dried over anhydrous Na₂SO₄ and concentrated under reduced pressure to obtain crude product, which was purified by silica gel column chromatography eluting with 30% EtOAc/PE to afford compound 4 (7.88 g, 89%) as a colorless oil. LC-MS: m/z=200.0 [M+H]⁺

### ethyl 2-(bromomethyl)-6-chloronicotinate (5)

A mixture of ethyl 6-chloro-2-methylnicotinate compound 4 (7.88 g, 39.5 mmol), NBS (7.74 g, 43.5 mmol), and AIBN (64.9 mg, 0.395 mmol) in CCl₄ (80 mL) was stirred under nitrogen atmosphere at reflux for 4 hours. The mixture was concentrated, and the residue was washed ether. The filtrate was concentrated. The residue was purified by silica gel column chromatography eluting with 25% EtOAc/PE to afford compound 5 (6.94 g, 63%) as a colorless oil. LC-MS: m/z=278.0 [M+H]⁺

### ethyl 2-chloro-5-oxo-6,7-dihydro-5H-cyclopenta[b]pyridine-6-carboxylate (6)

Step 1: To a mixture of diethyl malonate(8.0 g, 50 mmol) in THF (100 ml) was added portionwise NaH (60% in mineral oil, 2.0 g, 50.0 mmol) at room temperature, and the mixture was stirred under nitrogen atmosphere at room temperature for 0.5 hour. To the mixture was added the obtained oil compound 5 (6.49 g, 24.89 mmol) and the mixture was stirred under nitrogen atmosphere at room temperature for 12 hour. The mixture was poured into ice water, neutralized with 1 M HCl aqueous solution, acidified with diluted citric acid aqueous solution and extracted with EtOAc. The extract was washed with brine, dried over anhydrous Na₂SO₄ and concentrated under reduced pressure to obtain crude product, which was purified by silica gel column chromatography eluting with 30% EtOAc/PE to afford intermediate (6.58 g, 74%) as a colorless oil. LC-MS: m/z=358.0 [M+H]⁺

Step 2: To a suspension of NaH (60% in mineral oil, 0.88 g, 22.0 mmol) at room temperature was added intermediate obtained from previous step (6.49 g, 24.89 mmol) and the mixture was stirred under nitrogen atmosphere at room temperature for 12 hour. The mixture was poured into ice water, neutralized with 1 M HCl aqueous solution, acidified with diluted citric acid aqueous solution and extracted with EtOAc. The extract was washed with brine, dried over anhydrous Na₂SO₄ and concentrated under reduced pressure to obtain crude product, which was purified by silica gel column chromatography eluting with 30% EtOAc/PE to afford compound 6 (6.58 g, 74%) as a colorless oil. LC-MS: m/z=240.0 [M+H]⁺

### 2-hydroxy-6,7-dihydro-5H-cyclopenta[b]pyridin-5-one (8)

A mixture of compound 6 (4.4 g, 18.4 mmol) and phosphoric acid (50 ml) was stirred under nitrogen atmosphere at 180 degree for 3 hours. After cooling, the mixture was poured into ice water, and neutralized with 8 M NaOH aqueous solution and NaHCO3. The mixture was concentrated and diluted with EtOH, and the filtrate was concentrated. The resulting slid was washed with EtOH, and dried to give compound 7 (2.33 g, 85%) as a khaki solid. LC-MS: m/z=150.0 [M+H]⁺

### 6,7-dihydrospiro[cyclopenta[b]pyridine-5,4′-imidazolidine]-2,2′,5′(1H)-trione (9)

To a stirring solution of 4-methoxy-2,3-dihydroinden-1-one compound 7 (450 mg, 3 mmol) in formamide (10 mL) was added (NH₄)₂CO₃ (1.44 g, 15 mmol) followed by potassium cyanide (585 mg, 9 mmol) at room temperature. The reaction mixture was heated to 75° C. for 18 h in sealed tube. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 10% MeOH/DCM to afford compound 8 (200 g, 30%) as a yellow oil. LC-MS: m/z=220.1 [M+H]⁺

To a stirring solution of compound 8 (200 mg, 0.91 mmol) and intermediate 13 (270 mg, 0.91 mmol) in DMF (5 mL) was added K₂CO₃ (251 mg, 1.82 mmol) and stirred at room temperature for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 50% EtOAc/PE to afford compound 69-1 (13 mg, 3%) as an off-white solid. LC-MS: m/z=435.2 [M+H]⁺ at RT 8.57 (99.76% purity). ¹H NMR ((400 MHz, DMSO-d6) δ 8.65 (d, J=12.7 Hz, 1H), 7.53-7.08 (m, 7H), 6.20 (t, J=8.3 Hz, 1H), 4.78-4.48 (m, 2H), 4.51-4.01 (m, 2H), 3.70 (dd, J=16.0, 6.7 Hz, 1H), 2.80 (t, J=12.6 Hz, 2H), 2.55 (dd, J=16.0, 7.4 Hz, 1H), 2.16 (td, J=14.2, 8.4 Hz, 1H), 1.25-1.01 (m, 3H), 0.92 (d, J=4.5 Hz, 1H), 0.67-−0.06 (m, 4H).

### Example 70

### N-benzyl-N-(1-cyclopropylethyl)-2-(5′-(4,5-dihydro-1H-imidazol-2-yl)-2,5-dioxo-2′,3′-dihydrospiro[imidazolidine-4,1′-indene]-1-yl)acetamide (3)

A solution of N-benzyl-N-(1-cyclopropylethyl)-2-(5′-formyl-2,5-dioxo-2′,3′-dihydrospiro[imidazolidine-4,1′-indene]-1-yl)acetamide SM1 (29 mg, 0.066 mmol), SM2 (4.35 mg, 0.073 mmol), I₂ (21 mg, 0.083 mmol) and K₂CO₃ (27 mg, 0.2 mmol) dissolved with HOBu-t (5 mL) was stirred at 70° C. overnight. After consumption of the starting material (by TLC and LC-MS), the solvent from reaction mixture was removed under reduced pressure, the residue was diluted with brine and extracted with EA. Combined organic extracts were dried over anhydrous Na₂SO₄ and concentrated under reduced pressure to obtain crude product, which was purified by silica gel column chromatography to afford compound 3 (18 mg, 56%) as an off-white solid. TLC: 50% EA/PE

### 2-(5′-(1H-imidazol-2-yl)-2,5-dioxo-2′,3′-dihydrospiro[imidazolidine-4,1′-indene]-1-yl)-N-benzyl-N-(1-cyclopropylethyl)acetamide

A solution of SM3 (18 mg, 0.037 mmol), DIB (13 mg, 0.041 mmol) and K₂CO₃ (7 mg, 0.041 mmol) in DMSO (2 mL) was stirred overnight. By LC-MS, 50% SM3 was converted. The reaction mixture was diluted with sat NaCl aqueous and extracted with EtOAc. Combined organic extracts were dried over anhydrous Na₂SO₄ and concentrated under reduced pressure to obtain crude product, which was purified by Prep-HPLC [H₂O-ACN(0.05% TFA)] to afford compound 70-1 (8 mg, 45%) as a white liquid. ¹H NMR (400 MHz, DMSO-d₆): δ 14.73-14.68 (m, 1H), 8.95-8.90 (m, 1H), 7.98-7.25 (m, 10H), 4.75-4.05 (m, 4H), 3.20-2.94 (m, 2H), 2.64 (dd, J=13.7, 7.3 Hz, 1H), 2.55-2.28 (m, 1H), 2.28-1.78 (m, 1H), 1.25-1.16 (m, 3H), 1.15-0.95 (m, 1H), 0.78-0.20 (m, 4H). LC-MS: m/z=484.2 [M+H]⁺ (93.8% purity, 214 nm)

### Example 71

### 5-bromo-1-hydroxy-2,3-dihydro-1H-indene-1-carbonitrile (1)

To a stirring solution of SM1 (5 g, 23.7 mmol) in dry DCM (36 mL) was added TMSCN (4.7 g, 47.4 mmol) followed by Cu(OTf)₂ (429 mg, 1.185 mmol) at room temperature and stirred for 3 days. 1N HCl (14 mL) and ACN (14 mL) were added and then stirred at room temperature for 2 hours. After quenched with water (42 mL), the mixture was extracted with DCM (70 mL×3). Combined organic extracts were washed with brine, dried over anhydrous Na₂SO₄ and concentrated under reduced pressure to afford compound 1 (5.58 g, 99%) as a brown solid. ¹H NMR (300 MHz, CDCl₃) δ 7.64-7.34 (m, 3H), 3.28-3.10 (m, 1H), 3.01 (ddd, J=16.4, 8.3, 4.1 Hz, 1H), 2.75 (ddd, J=13.7, 8.2, 7.1 Hz, 1H), 2.63 (s, 1H), 2.51 (ddd, J=13.7, 7.7, 4.1 Hz, 1H).

### ethyl 5-bromo-1-hydroxy-2,3-dihydro-1H-indene-1-carbimidate (2)

To a stirring solution of compound 1 (1 g, 4.2 mmol) in dry EtOH (20 mL) was bubbled HCl gas at 0-5° C. The resulting mixture was stirred at 0-5° C. overnight. The solvent was removed under reduced pressure to afford the crude compound 2 (1.33 g, 98%) as a dark-blue solid. LC-MS: m/z=284/286 [M+H]⁺

### 5-bromo-4′-ethoxy-2,3-dihydro-2′H-spiro[indene-1,5′-oxazol]-2′-one (3)

To a stirring solution of compound 2 (1.328 g, 4.412 mmol) in DCM (40 mL) was added Et₃N (1.255 g, 12.426 mmol) followed by triphosgene (430 mg, 1.45 mmol) at 0-5° C. under N₂ and stirred at room temperature for 2 hours. The reaction mixture was diluted with water and extracted with DCM. Combined organic extracts were dried over anhydrous Na₂SO₄ and concentrated under reduced pressure to obtain crude compound 3 (1.29 g, 97%) as a dark oil. LC-MS: m/z=310/312 [M+H]⁺

### 5-bromo-2,3-dihydrospiro[indene-1,5′-ozazolidine]-2′,4′-dione (4)

To a stirring solution of compound 3 (1.291 g, 3.64 mmol) in THF (10 mL) was added a solution of Na₂CO₃ (0.742 g) in water (10 mL) at room temperature. After stirred at room temperature for 16 hours, the reaction mixture was diluted with water and extracted with EtOAc. The aqueous phase acidified to pH=1 and extracted with EtOAc. Combined organic extracts were dried over anhydrous Na₂SO₄ and concentrated under reduced pressure to obtain crude product, which was purified by silica gel column chromatography eluting with 10% MeOH/DCM to afford compound 2 (841 mg, 72%) as a dark blue solid. LC-MS: m/z=282/284 [M+H]⁺

To a stirring solution of compound 4 (250 mg, 0.887 mmol) in DMF (6 mL) was added intermediate 13 (263 mg, 0.887 mmol) followed by K₂CO₃ (245 mg, 1.774 mmol) at room temperature and stirred at room temperature overnight. EtOAc was added and then filtered. The filtrate was concentrated under reduced pressure to obtain crude product, which was purified by prep-HPLC eluting with ACN/H₂O containing 5% TFA to afford 71-1 (364 mg, 83%) as a white solid. LC-MS: m/z=497.1/499.1 [M+H]⁺ at RT 5.697 (95.599% purity). ¹H NMR (400 MHz, DMSO-d6): δ 7.90-7.15 (m, 8H), 4.90-4.15 (m, 4H), 3.77 (dd, J=15.9, 9.2 Hz, 0.6H), 3.52-3.40 (m, 0.4H), 3.28-2.98 (m, 2H), 2.68 (dd, J=14.4, 7.6 Hz, 2H), 1.35-1.10 (m, 3H), 1.02-0.88 (m, 1H), 0.50-0.10 (m, 4H).

### Example 72

### 1-(trimethylsilyloxy)-2,3-dihydro-1H-indene-1-carbonitrile (1)

To a stirring solution of 2,3-dihydroinden-1-one SM1 (1 g, 7.5 mmol) in toluene (10 mL), CH₃CN (5 mL), was added TMSCN (1.3 eq, 9.75 mmol), followed by ZnI₂ (0.03 eq, 2.25 mmol) at room temperature. The reaction mixture was heated to 50° C. for 16 h. After consumption of the starting material (by TLC), the reaction mixture was diluted with aq NaHCO₃ and extracted with EtOAc. Combined organic extracts were dried over anhydrous Na₂SO₄ and concentrated under reduced pressure to obtain crude product, which was purified by silica gel column chromatography eluting with 20% EA/PE to afford compound 1 (1.5 g, 88%). TLC: 20% EA/PE (R_{f}: 0.3)

### ethyl 1-hydroxy-2,3-dihydro-1H-indene-1-carbimidate hydrochloride (2)

A solution of 1-(trimethylsilyloxy)-2,3-dihydro-1H-indene-1-carbonitrile compound 1 (1.5 g, 6.5 mmol), in ethanol (10 mL) was cooled to 0° C. HCl (gas) was bubbled in for 3 hours. After consumption of the starting material (by TLC), the solvent was removed under reduced pressure. The residue was washed with ether to afford compound 2 (Ig, 64%). LC-MS: m/z=242 [M+H]⁺

### 2,3-dihydrospiro[indene-1,5′-ozazolidine]-2′,4′-dione (3)

To a stirring solution of compound 2 (1 g, 4.14 mmol) in dry THF (20 mL) was added Et₃N (1.25 g, 4.14 mmol) followed by phosgene (1.2 g, 12.4 mmol) at 0° C. and stirred at room temperature for 1 hour. The reaction mixture was diluted with 1N HCl, stirred at room temperature for 0.5 h, and extracted with EA. Combined organic extracts were dried over anhydrous Na₂SO₄ and concentrated under reduced pressure to obtain crude product, which was purified by silica gel column chromatography eluting with 30% EA/PE to afford compound 3 (500 mg, 60%). TLC: 30% EtOAc/Hexane (R_{f}: 0.4) LC-MS: m/z=204 [M+H]⁺

### tert-butyl 2-(2′,4′-dioxo-2,3-dihydrospiro[indene-1,5′-ozazolidine]-3′-yl)acetate (5)

To a stirring solution of 2,3-dihydrospiro[indene-1,5′-ozazolidine]-2′,4′-dione compound 3 (250 mg, 1.23 mmol) in DMF (10 mL) was added compound 4 (239 mg, 1.23 mmol), followed by K₂CO₃ (339 mg, 2.46 mmol) at room temperature and stirred at room temperature for 1 hour. The reaction mixture was diluted with water and extracted with EA. Combined organic extracts were dried over anhydrous Na₂SO₄ and concentrated under reduced pressure to obtain crude product, which was purified by silica gel column chromatography eluting with 30% EA/PE to afford compound 5 (300 mg, 76.9%). TLC: 30% EtOAc/Hexane (R_{f}: 0.5) LC-MS: m/z=318 [M+H]⁺

### 2-(2′,4′-dioxo-2,3-dihydrospiro[indene-1,5′-ozazolidine]-3′-yl)acetic acid (6)

To a stirring solution of tert-butyl 2-(2′,4′-dioxo-2,3-dihydrospiro[indene-1,5′-ozazolidine]-3′-yl)acetate compound 5 (300 mg, 0.95 mmol) in DCM (5 mL) was added TFA (5 mL) at room temperature and stirred at room temperature for 2 hours. The reaction mixture concentrated under reduced pressure to obtain crude compound 6 (200 mg, 81%). TLC: 50% EtOAc/Hexane (R_{f}: 0.2). LC-MS: m/z=262 [M+H]⁺

To a stirring solution of 2-(2′,4′-dioxo-2,3-dihydrospiro[indene-1,5′-ozazolidine]-3′-yl)acetic acid compound 6 (50 mg, 0.19 mmol), in DMF (5 mL) was added N-benzylpentan-3-amine compound 7 (33.6 mg, 0.19 mmol), followed by DIEA (49 mg, 0.38 mmol) and HATU (108 mg, 0.29 mmol), After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 50% EA/PE to afford 72-1 (30 mg, 37.5%). LC-MS: m/z=421 [M+H]⁺ at RT 1.75 (100% purity). ¹H NMR (300 MHz, DMSO) δ 7.65-7.08 (m, 9H), 4.67-4.34 (m, 5H), 4.28-3.58 (m, 1H), 3.26-2.98 (m, 2H), 2.78-2.58 (m, 1H), 1.46 (dd, J=14.3, 6.8 Hz, 4H), 0.78 (t, J=6.9 Hz, 6H).

### Example 73

### 1-(trimethylsilyloxy)-2,3-dihydro-1H-indene-1-carbonitrile (1)

To a stirring solution of 2,3-dihydroinden-1-one SM1 (1 g, 7.5 mmol) in Toluene (10 ml), CH₃CN (5 ml), was added TMSCN (1.3 eq, 9.75 mmol), followed by ZnI₂ (0.03 eq, 2.25 mmol) at room temperature. The reaction mixture was heated to 50° C. for 16 h. After consumption of the starting material (by TLC), the reaction mixture was diluted with aq NaHCO₃ and extracted with EtOAc. Combined organic extracts were dried over anhydrous Na₂SO₄ and concentrated under reduced pressure to obtain crude product, which was purified by silica gel column chromatography eluting with 20% EA/PE to afford compound 1 (1.5 g, 88%). TLC: 20% EA/PE (R_{f}: 0.3)

### ethyl 1-hydroxy-2,3-dihydro-1H-indene-1-carbimidate hydrochloride (2)

A solution of 1-(trimethylsilyloxy)-2,3-dihydro-1H-indene-1-carbonitrile compound 1 (1.5 g, 6.5 mmol), in ethanol (10 mL) was cooled to 0° C. HCl (gas) was bubbled in for 3 hours. After consumption of the starting material (by TLC), the solvent from reaction mixture was removed under reduced pressure. Obtained residue was washed with ether to afford compound 2 (Ig, 64%). LC-MS: m/z=242 [M+H]⁺

### 2,3-dihydrospiro[indene-1,5′-ozazolidine]-2′,4′-dione (3)

To a stirring solution of compound 2 (Ig, 4.14 mmol) in dry THF (20 mL) was added Et₃N (1.25 g, 4.14 mmol) followed by phosgene (1.2 g, 12.4 mmol) at 0° C. and stirred at room temperature for 1 hour. The reaction mixture was diluted with 1N HCl, stirred at room temperature for 0.5 h, and extracted with EA. Combined organic extracts were dried over anhydrous Na₂SO₄ and concentrated under reduced pressure to obtain crude product, which was purified by silica gel column chromatography eluting with 30% EA/PE to afford compound 3 (500 mg, 60%). TLC: 30% EtOAc/Hexane (R_{f}: 0.4). LC-MS: m/z=204 [M+H]⁺

### tert-butyl 2-(2′,4′-dioxo-2,3-dihydrospiro[indene-1,5′-ozazolidine]-3′-yl)acetate (5)

To a stirring solution of 2,3-dihydrospiro[indene-1,5′-ozazolidine]-2′,4′-dione compound 3 (250 mg, 1.23 mmol) in DMF (10 mL) was added compound 4 (239 mg, 1.23 mmol), followed by K₂CO₃ (339 mg, 2.46 mmol) at room temperature and stirred at room temperature for 1 hour. The reaction mixture was diluted with water and extracted with EA. Combined organic extracts were dried over anhydrous Na₂SO₄ and concentrated under reduced pressure to obtain crude product, which was purified by silica gel column chromatography eluting with 30% EA/PE to afford compound 5 (300 mg, 76.9%). TLC: 30% EtOAc/Hexane (R_{f}: 0.5). LC-MS: m/z=318 [M+H]⁺

### 2-(2′,4′-dioxo-2,3-dihydrospiro[indene-1,5′-ozazolidine]-3′-yl)acetic acid (6)

To a stirring solution of tert-butyl 2-(2′,4′-dioxo-2,3-dihydrospiro[indene-1,5′-ozazolidine]-3′-yl)acetate compound 5 (300 mg, 0.95 mmol) in DCM (5 mL) was added TFA (5 ml) at room temperature and stirred at room temperature for 2 hours. The reaction mixture concentrated under reduced pressure to obtain crude compound 6 (200 mg, 81%). TLC: 50% EtOAc/Hexane (R_{f}: 0.2). LC-MS: m/z=262 [M+H]⁺

### N-benzyl-N-(1-cyclobutylethyl)-2-(2′,4′-dioxo-2,3-dihydrospiro[indene-1,5′-ozazolidine]-3′-yl)acetamide

To a stirring solution of 2-(2′,4′-dioxo-2,3-dihydrospiro[indene-1,5′-ozazolidine]-3′-yl)acetic acid compound 6 (50 mg, 0.19 mmol), in DMF (5 mL) was added N-benzyl-1-cyclobutylethanamine intermediate 11 (36.2 mg, 0.19 mmol), followed by DIEA (49 mg, 0.38 mmol) and HATU (108 mg, 0.29 mmol), After consumption of the starting material (by TLC), the reaction mixture was diluted with water and extracted with EtOAc. Combined organic extracts were dried over anhydrous Na2SO4 and concentrated under reduced pressure to obtain crude product, which was purified by silica gel column chromatography eluting with 50% EA/PE to afford 73-1 (30 mg, 36.6%). LC-MS: m/z=433 [M+H]⁺ at RT 1.76 (100% purity). ¹H NMR (300 MHz, DMSO-d6) δ 7.64-6.96 (m, 9H), 4.80-4.20 (m, 5H), 3.99 (s, 1H), 3.25-2.98 (m, 2H), 2.64 (ddd, J=20.6, 9.6, 5.9 Hz, 1H), 2.00-1.40 (m, 6H), 1.11-0.86 (m, 3H).

### Example 74

### 1-(trimethylsilyloxy)-2,3-dihydro-1H-indene-1-carbonitrile (1)

To a stirring solution of 2,3-dihydro-1H-inden-1-one SM1 (1 g, 7.5 mmol) in toluene (10 mL), CH₃CN (5 mL), was added TMSCN (1.3 eq, 9.75 mmol), followed by ZnI₂ (0.03 eq, 2.25 mmol) at room temperature. The reaction mixture was heated to 50° C. for 16 h. After consumption of the starting material (by TLC), the reaction mixture was diluted with aq NaHCO₃ and extracted with EtOAc. Combined organic extracts were dried over anhydrous Na₂SO₄ and concentrated under reduced pressure to obtain crude product, which was purified by silica gel column chromatography eluting with 20% EA/PE to afford compound 1 (1.5 g, 88%). TLC: 20% EA/PE (R_{f}: 0.3)

### ethyl 1-hydroxy-2,3-dihydro-1H-indene-1-carbimidate hydrochloride (2)

A solution of 1-(trimethylsilyloxy)-2,3-dihydro-1H-indene-1-carbonitrile compound 1 (1.5 g, 6.5 mmol), in ethanol (10 mL) was cooled to 0° C. HCl (gas) was charged for 3 hours. After consumption of the starting material (by TLC), the solvent from reaction mixture was removed under reduced pressure. Obtained residue was washed with ether to afford compound 2 (Ig, 64%). LC-MS: m/z=242 [M+H]⁺

### 2,3-dihydrospiro[indene-1,5′-ozazolidine]-2′,4′-dione (3)

To a stirring solution of compound 2 (Ig, 4.14 mmol) in dry THF (20 mL) was added Et₃N (1.25 g, 4.14 mmol) followed by phosgene (1.2 g, 12.4 mmol) at 0° C. and stirred at room temperature for 1 hour. The reaction mixture was diluted with 1N HCl, stirred at room temperature for 0.5 hour, and extracted with EA. Combined organic extracts were dried over anhydrous Na₂SO₄ and concentrated under reduced pressure to obtain crude product, which was purified by silica gel column chromatography eluting with 30% EA/PE to afford compound 3 (500 mg, 60%). TLC: 30% EtOAc/Hexane (R_{f}: 0.4). LC-MS: m/z=204 [M+H]⁺

### tert-butyl 2-(2′,4′-dioxo-2,3-dihydrospiro[indene-1,5′-ozazolidine]-3′-yl)acetate (5)

To a stirring solution of 2,3-dihydrospiro[indene-1,5′-ozazolidine]-2′,4′-dione compound 3 (250 mg, 1.23 mmol) in DMF (10 mL) was added compound 4 (239 mg, 1.23 mmol), followed by K₂CO₃ (339 mg, 2.46 mmol) at room temperature and stirred at room temperature for 1 hour. The reaction mixture was diluted with water and extracted with EA. Combined organic extracts were dried over anhydrous Na₂SO₄ and concentrated under reduced pressure to obtain crude product, which was purified by silica gel column chromatography eluting with 30% EA/PE to afford compound 5 (300 mg, 76.9%). TLC: 30% EtOAc/Hexane (R_{f}: 0.5). LC-MS: m/z=318 [M+H]⁺

### 2-(2′,4′-dioxo-2,3-dihydrospiro[indene-1,5′-ozazolidine]-3′-yl)acetic acid (6)

To a stirring solution of tert-butyl 2-(2′,4′-dioxo-2,3-dihydrospiro[indene-1,5′-ozazolidine]-3′-yl)acetate compound 5 (300 mg, 0.95 mmol) in DCM (5 mL) was added TFA (5 mL) at room temperature and stirred at room temperature for 2 hours. The reaction mixture concentrated under reduced pressure to obtain crude compound 6 (200 mg, 81%). TLC: 50% EtOAc/Hexane (R_{f}: 0.2). LC-MS: m/z=262 [M+H]⁺

To a stirring solution of 2-(2′,4′-dioxo-2,3-dihydrospiro[indene-1,5′-ozazolidine]-3′-yl)acetic acid compound 6 (50 mg, 0.19 mmol), in DMF (5 mL) was added 1-cyclopropyl-N-(4-fluorobenzyl)ethanamine intermediate 34 (36.6 mg, 0.19 mmol), followed by DIEA (49 mg, 0.38 mmol) and HATU (108 mg, 0.29 mmol), After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 50% EA/PE to afford 74-1 (30 mg, 36.6%). LC-MS: m/z=437 [M+H]⁺ at RT 1.74 (100% purity). ¹H NMR (300 MHz, DMSO) δ 7.58-7.01 (m, 8H), 4.81-4.24 (m, 4H), 3.81-3.62 (m, 1H), 3.13 (dd, J=15.9, 8.1 Hz, 1H), 2.65 (s, 1H), 1.31-1.07 (m, 3H), 0.95 (s, 2H), 0.59-0.05 (m, 4H).

### Example 75

### 4-methoxy-1-(trimethylsilyloxy)-2,3-dihydro-1H-indene-1-carbonitrile (2)

To a stirring solution of 4-methoxy-2,3-dihydroinden-1-one SM1 (2 g, 12.3 mmol) in toluene (10 mL), CH₃CN (5 ml), was added TMSCN (1.3 eq, 16 mmol), followed by ZnI₂ (0.03 eq, 0.37 mmol) at room temperature. The reaction mixture was heated to 50° C. for 16 h. After consumption of the starting material (by TLC), the reaction mixture was diluted with aq NaHCO₃ and extracted with EtOAc. Combined organic extracts were dried over anhydrous Na₂SO₄ and concentrated under reduced pressure to obtain crude product, which was purified by silica gel column chromatography eluting with 20% EA/PE to afford compound 1 (1.9 g, 59.3%). TLC: 20% EA/PE (R_{f}: 0.3)

### ethyl 1-hydroxy-4-methoxy-2,3-dihydro-1H-indene-1-carbimidate hydrochloride (2)

A solution of 4-methoxy-1-(trimethylsilyloxy)-2,3-dihydro-1H-indene-1-carbonitrile compound 1 (500 mg, 1.9 mmol), in ethanol (10 mL) was cooled to 0° C. HCl (gas) was charged for 3 hours. After consumption of the starting material (by TLC), the solvent from reaction mixture was removed under reduced pressure. Obtained residue was washed with ether to afford compound 2 (460 mg, 88%). LC-MS: m/z=272 [M+H]⁺

### 4-methoxy-2,3-dihydrospiro[indene-1,5′-ozazolidine]-2′,4′-dione (3)

To a stirring solution of compound 2 (200 mg, 0.74 mmol) in dry THF (10 mL) was added Et₃N (5 eq, 3.7 mmol) followed by phosgene (3 eq, 2.2 mmol) at 0° C. and stirred at room temperature for 1 hour. The reaction mixture was diluted with 1N HCl, stirred at room temperature for 0.5 h, and extracted with EA. Combined organic extracts were dried over anhydrous Na₂SO₄ and concentrated under reduced pressure to obtain crude product, which was purified by silica gel column chromatography eluting with 30% EA/PE to afford compound 3 (150 mg, 87%). TLC: 30% EtOAc/Hexane (R_{f}: 0.5). LC-MS: m/z=234 [M+H]⁺

To a stirring solution of 4-methoxy-2,3-dihydrospiro[indene-1,5′-ozazolidine]-2′,4′-dione compound 3 (250 mg, 1 mmol) in DMF (10 mL) was added intermediate 13 (315 mg, 1 mmol), followed by K₂CO₃ (295 mg, 2 mmol) at room temperature and stirred at room temperature for 1 hour. The reaction mixture was diluted with water and extracted with EA. Combined organic extracts were dried over anhydrous Na₂SO₄ and concentrated under reduced pressure to obtain crude product, which was purified by silica gel column chromatography to afford 75-1 (200 mg, 41.7%) as an off-white solid. LC-MS: m/z=449 [M+H]⁺ at RT 1.74 (100% purity). ¹H NMR (300 MHz, DMSO) δ 7.50-7.00 (m, 8H), 4.77 (s, 4H), 3.83 (s, 3H), 3.10-2.87 (m, 2H), 2.73-2.55 (m, 1H), 1.16 (dd, J=19.9, 4.9 Hz, 3H), 1.03-0.74 (m, 2H), 0.57-0.07 (m, 4H).

### Example 76

### (4-(bromomethyl)phenyl)(methyl)sulfane (1)

To a stirring solution of (4-(methylthio)phenyl)methanol SM 1 (500 mg, 3.2 mmol) in DCM (10 mL) was added PBr₃ (866 mg, 3.2 mmol), and stirred at room temperature for 2 hours. The reaction mixture was diluted with water and extracted with EA. Combined organic extracts were dried over anhydrous Na₂SO₄ and concentrated under reduced pressure to obtain crude product, which was purified by silica gel column chromatography with 20% EA/PE to afford compound 1 (400 mg, 57.8%). TLC:20% EtOAc/Hexane (R_{f}: 0.5)

### (S)-1-cyclobutyl-N-(4-(methylthio)benzyl)ethanamine (3)

To a stirring solution of (4-(bromomethyl)phenyl)(methyl)sulfane compound 1 (50 mg, 0.23 mmol) in DCM (10 mL), was added (S)-1-cyclobutylethanamine compound 2 (22.7 mg, 0.23 mmol), followed by DIEA (59 mg, 0.46 mmol) at room temperature. The reaction mixture stirred at room temperature for 16 h. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 30% EA/PE to afford compound 3 (40 mg, 73%). TLC: 30% EA/PE (R_{f}: 0.4). LC-MS: m/z=236 [M+H]⁺

To a stirring solution of (S)-1-cyclobutyl-N-(4-(methylthio)benzyl)ethanamine compound 3 (35 mg, 0.15 mmol), in DMF (5 mL) was added (S)-2-(2,5-dioxo-2′,3′-dihydrospiro[imidazolidine-4,1′-indene]-1-yl)acetic acid intermediate 9 (40 mg, 0.15 mmol), followed by DIEA (38.7 mg, 0.3 mmol) and HATU (87.4 mg, 0.23 mmol), After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 30% EA/PE to afford 76-1 (20 mg, 20%). LC-MS: m/z=478 [M+H]⁺ at RT 1.72 (100% purity). ¹H NMR (300 MHz, DMSO) δ 8.80 (d, J=14.2 Hz, 1H), 7.46-7.04 (m, 8H), 4.38 (d, J=16.4 Hz, 5H), 3.04 (d, J=5.1 Hz, 2H), 2.62-2.52 (m, 1H), 2.44 (d, J=7.4 Hz, 4H), 2.30-2.14 (m, 1H), 1.97-1.81 (m, 1H), 1.70 (s, 4H), 0.93 (dd, J=30.1, 6.5 Hz, 3H).

### Example 77

### N-benzyl-N-(1-cyclopropylethyl)-2-(2,5-dioxo-5′-(2H-1,2,3-triazol-2-yl)-2′,3′-dihydrospiro[imidazolidine-4,1′-indene]-1-yl)acetamide

To a mixture of 1H-1,2,3-triazole SM (36 mg, 0.5 mmol), 2-(2,5-dioxo-2′,3′-dihydrospiro[imidazolidine-4,1′-indene]-1-yl)acetic acid intermediate 20 (100 mg, 0.2 mmol), (1S,2S)—N1,N2-dimethylcyclohexane-1,2-diamine Ligand (6 mg, 0.04 mmol), CuI (4 mg, 0.02 mmol) and Cs₂CO₃ (130 mg, 0.2 mmol) was added NMP (2 mL) and water (1 drop). The mixture was degassed and sealed, which was stirred at 170° C. for 1 hour under microwave. The reaction mixture was diluted with ethyl acetate (30 mL) and washed with water (10 mL). The organic extracts was concentrated under reduced pressure to obtain crude product, which was purified by Prep-HPLC to afford 77-1 (7.2 mg, 7%) and 77-2 (6.1 mg, 6%) as off-white solids. LC-MS 82-1: m/z=485.2 [M+H]⁺ at RT 4.16 (98.18% purity). ¹H NMR 82-1 (300 MHz, CD₃OD-d4): δ 8.54 (s, 1H), 7.97-7.58 (m, 4H), 7.51-7.35 (m, 2H), 7.32-7.09 (m, 3H), 4.82-4.69 (m, 2H), 4.55-4.28 (m, 2H), 3.88-3.73 (m, 1H), 3.27-3.10 (m, 2H), 2.88-2.70 (m, 1H), 2.52-2.30 (m, 1H), 1.35-1.12 (m, 3H), 0.98-0.93 (m, 1H), 0.67-0.47 (m, 1H), 0.42-0.15 (m, 3H). LC-MS 82-2: m/z=485.2 [M+H]⁺ at RT 4.69 (99.06% purity). ¹H NMR 82-2 (300 MHz, CD₃OD-d4): δ 8.07-7.83 (m, 2H), 7.91 (s, 2H), 7.57-7.12 (m, 6H), 4.85-4.70 (m, 2H), 4.65-4.28 (m, 2H), 3.84-3.80 (m, 1H), 3.28-3.08 (m, 2H), 2.91-2.69 (m, 1H), 2.42-2.38 (m, 1H), 1.39-1.15 (m, 3H), 0.96-0.92 (m, 1H), 0.58-0.54 (m, 1H), 0.47-0.18 (m, 3H).

### Example 78

### N—((S)-1-cyclobutylethyl)-N-(4-fluorobenzyl)-2-methylpropane-2-sulfinamide (1)

To a stirring solution of N—((S)-1-cyclobutylethyl)-2-methylpropane-2-sulfinamide Intermediate 16 (329 mg, 1.5 mmol) in DMF (2 mL) was added NaH (120 mg, 3 mmol) at 0° C. and stirred at 0° C. for 30 min. Then 1-(bromomethyl)-4-fluorobenzene SM (283 mg, 1.5 mmol) was added and stirred at room temperature for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 25% EtOAc/Hexane to afford compound 1 (0.32 g, 68%)

### (S)-1-cyclobutyl-N-(4-fluorobenzyl)ethanamine hydrochloride (2)

To a stirring solution of compound 1 (0.32 g, 1.03 mmol) in MeOH (10 mL) was added 2N HCl in dioxane (2 mL) and stirred at room temperature for 30 min. The reaction mixture was concentrated under reduced pressure to obtain compound 2 (213 mg, 100%) as a white solid used for next step directly. LC-MS: m/z=208.2 [M+H]⁺

To a stirring solution of compound 2 (38 mg, 0.154 mmol) and intermediate 9 (40 mg, 0.154 mmol) in DMF (1 mL) was added HATU (88 mg, 0.23 mmol) followed by DIPEA (40 mg, 0.31 mmol) and stirred at room temperature for 1 hour. The reaction mixture was diluted with water and extracted with EtOAc. Combined organic extracts were dried over anhydrous Na2SO4 and concentrated under reduced pressure to obtain crude product, which was purified by prep-hplc to afford compound 78-1 (26 mg, 38%) as an off-white solid. LC-MS: m/z=450.2 [M+H]⁺ at RT 4.95 (100% purity). ¹H NMR (300 MHz, DMSO-d6) δ 8.79 (d, J=13.6 Hz, 1H), 7.72-6.96 (m, 8H), 4.65-4.20 (m, 4H), 4.11-3.94 (m, 1H), 3.04 (dd, J=12.3, 6.6 Hz, 2H), 2.60-2.51 (m, 1H), 2.39 (s, 1H), 2.24 (dd, J=15.0, 5.6 Hz, 1H), 1.92 (s, 1H), 1.79-1.34 (m, 5H), 0.94 (dd, J=31.8, 6.6 Hz, 3H).

### Example 79

### (S)-4-((1-cyclobutylethylamino)methyl)benzonitrile (1)

To a stirring solution of 4-(bromomethyl)benzonitrile SM1 (196 mg, 1 mmol) in DCM/MeOH (10 mL/10 mL, v/v) was added (S)-1-cyclobutylethanamine hydrochloride SM2 (135 mg, 1 mmol) followed by DIPEA (258 mg, 2 mmol) at room temperature. The reaction mixture was heated to 60° C. for 16 h. After consumption of the starting material (by TLC), the reaction mixture was concentrated under reduced pressure. The obtained residue was purified by silica gel column chromatography eluting with 10% MeOH/DCM to afford compound 1 (0.2 g, 93%) as a colorless oil. LC-MS: m/z=215.1 [M+H]⁺

To a stirring solution of compound 1 (82 mg, 0.38 mmol) and (S)-2-(2,5-dioxo-2′,3′-dihydrospiro[imidazolidine-4,1′-indene]-1-yl)acetic acid intermediate 9 (100 mg, 0.38 mmol) in DCM (20 mL) was added Et₃N (77 mg, 0.76 mmol) followed by T3P (50% in EtOAc) (0.49 g, 0.76 mmol) and stirred at room temperature for 1 hour. The reaction mixture was diluted with water and extracted with DCM. Combined organic extracts were dried over anhydrous Na₂SO₄ and concentrated under reduced pressure to obtain crude product, which was purified by silica gel column chromatography eluting with 5% MeOH/DCM to afford compound 79-1 (80 mg, 46%) as an off-white solid. LC-MS: m/z=457.2 [M+H]⁺ at RT 4.61 (95.14% purity). ¹H NMR (300 MHz, DMSO-d6) δ 8.80 (d, J=10.9 Hz, 1H), 7.87-7.54 (m, 2H), 7.52 (d, J=8.1 Hz, 1H), 7.41-7.19 (m, 5H), 4.67-4.23 (m, 4H), 4.03 (dd, J=10.2, 6.5 Hz, 1H), 3.04 (t, J=7.2 Hz, 2H), 2.61-2.51 (m, 1H), 2.38 (s, 1H), 2.28-2.17 (m, 1H), 1.92 (s, 1H), 1.75-1.50 (m, 5H), 0.94 (dd, J=37.8, 6.5 Hz, 3H).

### Example 80

### 4-((N—((S)-1-cyclobutylethyl)-2-((S)-2,5-dioxo-2′,3′-dihydrospiro[imidazolidine-4,1′-indene]-1-yl)acetamido)methyl)benzamide (1)

To a stirring solution of N-(4-cyanobenzyl)-N—((S)-1-cyclobutylethyl)-2-((S)-2,5-dioxo-2′,3′-dihydrospiro[imidazolidine-4,1′-indene]-1-yl)acetamide 84-1 (56 mg, 0.123 mmol) in MeOH (8 mL) was added NaOH (10 mg, 0.246 mmol) followed by 30% H₂O₂ (0.5 mL) at 0° C. The reaction mixture was stirred at room temperature for 3 hours. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 10% MeOH/DCM to afford compound 80-1 (22 mg, 38%) as an off-white solid. LC-MS: m/z=475.2 [M+H]⁺ at RT 6.76 (98.94% purity). ¹H NMR (300 MHz, DMSO-d6) δ 8.80 (d, J=12.5 Hz, 1H), 7.90 (dd, J=22.1, 13.5 Hz, 2H), 7.76 (d, J=8.2 Hz, 1H), 7.57-6.75 (m, 7H), 4.72-4.20 (m, 4H), 4.03 (dd, J=21.7, 11.5 Hz, 1H), 3.04 (dd, J=12.2, 6.8 Hz, 2H), 2.63-2.52 (m, 1H), 2.46-2.39 (m, 1H), 2.29-2.12 (m, 1H), 1.92 (s, 1H), 1.75-1.50 (m, 5H), 0.94 (dd, J=32.3, 6.5 Hz, 3H).

### Example 81

To a stirring solution of 53-1 (75 mg, 0.168 mmol) in MeCN/DCM (0.5 mL/0.5 mL) were added Ag₂O (47 mg, 0.2 mmol) and KI (42 mg, 0.168 mmol) followed by MeI (24 mg, 0.168 mmol) at room temperature. The reaction mixture was stirred for 2 hours at room temperature. The solvent was removed under reduced pressure. The residue was purified by Prep-HPLC to give compound 81-1 (5 mg, 6.5%) as an off-white solid.

LC-MS: m/z=462 [M+H]⁺ at RT 1.59 (91.5% purity). ¹H NMR (400 MHz, DMSO-d6): δ 8.78 (d, J=16.9 Hz, 1H), 7.49-7.14 (m, 7H), 4.74 (s, 1H), 4.62 (s, 1H), 4.41 (s, 2H), 4.41-4.19 (m, 2H), 3.76-3.67 (m, 1H), 3.29 (s, 3H), 3.03 (dd, J=13.2, 6.5 Hz, 2H), 2.55 (d, J=6.9 Hz, 1H), 2.28-2.13 (m, 1H), 1.20-1.05 (m, 3H), 0.94-0.86 (m, 1H), 0.53-0.08 (m, 4H).

### Example 82

### N—((S)-1-cyclobutylethyl)-2-methyl-N-(4-methylbenzyl)propane-2-sulfinamide (1)

To a solution of N—((S)-1-cyclobutylethyl)-2-methylpropane-2-sulfinamide Intermediate-16 (200 mg, 1 mol) in DMF (2 mL) were added NaH (80 mg, 2 mmol) at 0° C., the reaction mixture was stirred at 0° C. for 10 min, then 1-(bromomethyl)-4-methylbenzene SM1 (204 mg, 1.1 mol) was added, the mixture was allowed to warm to room temperature an stirred for ¹H. The reaction mixture was quenched with water and extracted with EtOAc. Combined organic extracts were dried over anhydrous Na₂SO₄ and concentrated under reduced pressure to obtain crude product, which was purified by silica gel column chromatography using PE/EtOAc (4:1) to afford compound 1 (180 mg, 59%) as an oil. LC-MS: m/z=288 [M+H]⁺

### (S)-1-cyclobutyl-N-(4-methylbenzyl)ethanamine hydrochloride (2)

A solution of compound 1 (180 mg, 0.59 mmol) in dioxane (2 mL) containing HCl (g) was stirred at room temperature for 2 hours. The solvent was removed under reduced pressure to obtain crude product, which was used to the next step without purification. LC-MS: m/z=204 [M+H]⁺

To a mixture of compound 2 (36 mg, 0.15 mmol), intermediate-9 (40 mg, 0.15 mmol) and DIPEA (43 mg, 0.33 mmol) in DMF (1 mL) was added HATU (70 mg, 0.185 mmol). The mixture was stirred at room temperature for 2 hours. The mixture was diluted with water and extracted with EtOAc. Combined organic extracts were washed with brine and dried over anhydrous Na₂SO₄, and concentrated under reduced pressure. The residue was purified by flash chromatography eluted with PE/EtOAc to afford compound 82-1 (15 mg, 22%) as an off-white solid. LC-MS: m/z=446 [M+H]⁺ at RT 1.72 (94.68% purity). ¹H NMR (400 MHz, DMSO-d6): δ 8.80 (d, J=20.6 Hz, 1H), 7.43-7.02 (m, 8H), 4.65-4.19 (m, 4H), 4.13-3.92 (m, 1H), 3.05 (dd, J=14.1, 7.0 Hz, 2H), 2.56 (dd, J=12.9, 6.4 Hz, 1H), 2.42 (s, 1H), 2.32-2.13 (m, 4H), 1.93 (s, 1H), 1.75-1.40 (m, 5H), 0.94 (dd, J=37.2, 6.6 Hz, 3H).

### Example 83

### N—((S)-1-cyclobutylethyl)-N-(4-methoxybenzyl)-2-methylpropane-2-sulfinamide (1)

To a solution of N—((S)-1-cyclobutylethyl)-2-methylpropane-2-sulfinamide Intermediate-16 (300 mg, 1.48 mol) in DMF (3 mL) were added NaH (120 mg, 2.96 mmol) at 0° C., the reaction mixture was stirred at 0° C. for 10 min, then 1-(bromomethyl)-4-methoxybenzene SM1 (260 mg, 1.6 mol) was added, the mixture was allowed to warm to room temperature an stirred for 1 hour. The reaction mixture was quenched with water and extracted with EtOAc. Combined organic extracts were dried over anhydrous Na₂SO₄ and concentrated under reduced pressure to obtain crude product, which was purified by silica gel column chromatography using PE/EtOAc (4:1) to afford compound 1 (200 mg, 42%) as an oil. LC-MS: m/z=324 [M+H]⁺

### (S)-1-cyclobutyl-N-(4-methoxybenzyl)ethanamine hydrochloride (2)

A solution of compound 1 (200 mg, 0.62 mmol) in dioxane (2 mL) containing HCl (g) was stirred at room temperature for 2 hours. The solvent was removed under reduced pressure to obtain crude product, which was used to the next step without purification. LC-MS: m/z=220 [M+H]⁺

To a mixture of compound 2 (38 mg, 0.15 mmol), intermediate-9 (40 mg, 0.15 mmol) and DIPEA (44 mg, 0.34 mmol) in DMF (1 mL) was added HATU (70 mg, 0.185 mmol). The mixture was stirred at room temperature for 2 hours. The mixture was diluted with water and extracted with EtOAc. Combined organic extracts were washed with brine and dried over anhydrous Na₂SO₄, and concentrated under reduced pressure. The residue was purified by flash chromatography eluted with PE/EtOAc to afford compound 83-1 (25 mg, 35%) as an off-white solid. LC-MS: m/z=462 [M+H]⁺ at RT 1.65 (97.8% purity). ¹H NMR (400 MHz, DMSO-d6): δ 8.80 (d, J=19.8 Hz, 1H), 7.40-7.21 (m, 5H), 7.13 (d, J=8.5 Hz, 1H), 6.93 (d, J=8.6 Hz, 1H), 6.83 (d, J=8.6 Hz, 1H), 4.63-4.20 (m, 4H), 4.11 (d, J=16.5 Hz, 1H), 4.04-3.89 (m, 1H), 3.73 (d, J=11.5 Hz, 3H), 3.05 (q, J=7.0 Hz, 2H), 2.57 (dd, J=12.7, 6.2 Hz, 1H), 2.42 (s, 1H), 2.23 (td, J=13.6, 6.6 Hz, ¹H), 1.94 (s, 1H), 1.78-1.42 (m, 5H), 0.94 (dd, J=36.8, 6.6 Hz, 3H).

### Example 84

### N—((S)-1-cyclobutylethyl)-2-methyl-N-(3-methylbenzyl)propane-2-sulfinamide (1)

To a solution of N—((S)-1-cyclobutylethyl)-2-methylpropane-2-sulfinamide Intermediate-16 (200 mg, 1 mol) in DMF (2 mL) were added NaH (80 mg, 2 mmol) at 0° C., the reaction mixture was stirred at 0° C. for 10 min, then 1-(bromomethyl)-3-methylbenzene SM1 (203 mg, 1.1 mol) was added, the mixture was allowed to warm to room temperature an stirred for 1 hour. The reaction mixture was quenched with water and extracted with EtOAc. Combined organic extracts were dried over anhydrous Na₂SO₄ and concentrated under reduced pressure to obtain crude product, which was purified by silica gel column chromatography using PE/EtOAc (4:1) to afford compound 1 (170 mg, 59%) as an oil. LC-MS: m/z=308 [M+H]⁺

### (S)-1-cyclobutyl-N-(3-methylbenzyl)ethanamine hydrochloride (2)

A solution of compound 1 (170 mg, 0.55 mmol) in dioxane (2 mL) containing HCl (g) was stirred at room temperature for 2 hours. The solvent was removed under reduced pressure to obtain crude product, which was used to the next step without purification. LC-MS: m/z=204 [M+H]⁺

To a mixture of compound 2 (36 mg, 0.15 mmol), intermediate-9 (40 mg, 0.15 mmol) and DIPEA (43 mg, 0.33 mmol) in DMF (1 mL) was added HATU (70 mg, 0.185 mmol). The mixture was stirred at room temperature for 2 hours. The mixture was diluted with water and extracted with EtOAc. Combined organic extracts were washed with brine and dried over anhydrous Na₂SO₄, and concentrated under reduced pressure. The residue was purified by flash chromatography eluted with PE/EtOAc to afford compound 84-1 (22 mg, 33%) as an off-white solid. LC-MS: m/z=446 [M+H]⁺ at RT 1.72 (94.8% purity). ¹H NMR (400 MHz, DMSO-d6): δ 8.80 (d, J=21.6 Hz, 1H), 7.41-7.21 (m, 4H), 7.20-7.05 (m, 2H), 6.98 (dd, J=14.8, 7.2 Hz, 1H), 4.65-4.21 (m, 4H), 4.11 (d, J=16.6 Hz, 1H), 4.00 (d, J=10.6 Hz, 1H), 3.05 (dd, J=13.5, 6.7 Hz, 2H), 2.56 (d, J=9.1 Hz, 1H), 2.41 (s, 1H), 2.33-2.15 (m, 4H), 1.93 (s, 1H), 1.72-1.64 (m, 5H), 0.94 (dd, J=37.6, 6.5 Hz, 3H)

### Example 85

### N—((S)-1-cyclobutylethyl)-2-methyl-N-(2-methylbenzyl)propane-2-sulfinamide (1)

To a solution of N—((S)-1-cyclobutylethyl)-2-methylpropane-2-sulfinamide Intermediate-16 (200 mg, 1 mol) in DMF (2 mL) were added NaH (80 mg, 2 mmol) at 0° C., the reaction mixture was stirred at 0° C. for 10 min, then 1-(bromomethyl)-2-methylbenzene SM1 (204 mg, 1.1 mol) was added, the mixture was allowed to warm to room temperature an stirred for ¹H. The reaction mixture was quenched with water and extracted with EtOAc. Combined organic extracts were dried over anhydrous Na₂SO₄ and concentrated under reduced pressure to obtain crude product, which was purified by silica gel column chromatography using PE/EtOAc (4:1) to afford compound 1 (180 mg, 59%) as an oil. LC-MS: m/z=288 [M+H]⁺

### (S)-1-cyclobutyl-N-(2-methylbenzyl)ethanamine hydrochloride (2)

A solution of compound 1 (180 mg, 0.59 mmol) in dioxane (2 mL) containing HCl (g) was stirred at room temperature for 2 hours. The solvent was removed under reduced pressure to obtain crude product, which was used to the next step without purification. LC-MS: m/z=204 [M+H]⁺

To a mixture of compound 2 (36 mg, 0.15 mmol), intermediate-9 (40 mg, 0.15 mmol) and DIPEA (43 mg, 0.33 mmol) in DMF (1 mL) was added HATU (70 mg, 0.185 mmol). The mixture was stirred at room temperature for 2 hours. The mixture was diluted with water and extracted with EtOAc. Combined organic extracts were washed with brine and dried over anhydrous Na₂SO₄, and concentrated under reduced pressure. The residue was purified by flash chromatography eluted with PE/EtOAc to afford compound 85-1 (18 mg, 27%) as an off-white solid. LC-MS: m/z=446 [M+H]⁺ at RT 1.70 (95.1% purity). ¹H NMR (400 MHz, DMSO-d6): δ 8.77 (d, J=22.6 Hz, 1H), 7.38-7.04 (m, 7H), 6.93 (s, 1H), 4.68-4.32 (m, 3H), 4.29-4.15 (m, 1H), 4.03 (t, J=17.7 Hz, 1H), 3.04 (d, J=5.2 Hz, 2H), 2.56 (d, J=5.6 Hz, 1H), 2.44 (s, 1H), 2.35-2.13 (m, 4H), 1.92 (s, 1H), 1.74 (d, J=17.9 Hz, 5H), 1.00-0.80 (m, 3H).

### Example 86

### 1-(azidomethyl)-2,4-dimethoxybenzene (1)

To a stirring solution of (2,4-dimethoxyphenyl)methanol SM1 (2.9 g, 17.3 mmol) and DBU (3.15 g, 20.7 mmol) in dry toluene (50 mL) was added DPPA (5.7 g, 20.7 mmol) at 0° C. The reaction mixture was then stirred at 0° C. for 2 hours and concentrated under reduced pressure to obtain crude product, which was purified by silica gel column chromatography eluting with 12% EtOAc/Hexane to afford compound 1 (3.2 g, 96%).

TLC: 25% EtOAc/Hexane (R_{f}: 0.5)

### N—((S)-1-cyclobutylethyl)-2-methyl-N-(prop-2-ynyl)propane-2-sulfinamide (2)

To a stirring solution of N—((S)-1-cyclobutylethyl)-2-methylpropane-2-sulfinamide Intermediate-16 (2.9 g, 14.3 mmol) in dry DMF (50 ml) was added NaH (60%, 1.72 g, 43 mmol) at 0° C. The reaction mixture was stirred at room temperature for 0.5 h then recooled and treated with 3-bromoprop-1-yne (3 g, 25.7 mmol) at 0° C. The reaction mixture was stirred at room temperature for 2 hours then quenched with crushed ice and extracted with EtOAc. Combined organic extracts were dried over anhydrous Na₂SO₄ and concentrated and purified by silica gel column chromatography eluting with 50% EtOAc/Hexane to afford compound 2 (2 g, 84%) and 0.9 g of Intermediate-16 recovered.

TLC: 50% EtOAc/Hexane (R_{f}: 0.2)

### N—((R)-1-cyclobutylethyl)-N-((1-(2,4-dimethoxybenzyl)-1H-1,2,3-triazol-4-yl)methyl)-2-methylpropane-2-sulfinamide (3)

To a stirring solution of compound 1 (280 mg, 1.45 mmol) and compound 2 (350 mg, 1.45 mmol) in DMF (10 ml) was added aqueous sodium ascorbate (1M, 0.15 ml, 0.15 mmol) and aqueous CuSO₄ (1M, 0.15 ml, 0.15 mmol) at room temperature. The reaction mixture was stirred at room temperature for 2 hours then quenched with H₂O and extracted with EtOAc. Combined organic extracts were dried over anhydrous Na₂SO₄ and concentrated and purified by silica gel column chromatography eluting with 3% MeOH/DCM to afford compound 3 (500 mg, 79%). TLC: 5% MeOH/DCM (R_{f}: 0.3)

### (R)-1-cyclobutyl-N-((1-(2,4-dimethoxybenzyl)-1H-1,2,3-triazol-4-yl)methyl)ethanamine (4)

To a stirring solution of compound 3 (500 mg, 1.15 mmol) in 1,4-Dooxane (10 mL) was bubbled in HCl(g) for 10 mins (detected by TLC), then concentrated and purified by silica gel column chromatography eluting with 4% MeOH/DCM to afford compound 4 (380 mg, 99%). TLC: 5% MeOH/DCM (R_{f}: 0.15). LC-MS: m/z=331.2 [M+H]⁺

### N—((S)-1-cyclobutylethyl)-N-((1-(2,4-dimethoxybenzyl)-1H-1,2,3-triazol-4-yl)methyl)-2-((S)-2,5-dioxo-2′,3′-dihydrospiro[imidazolidine-4,1′-indene]-1-yl)acetamide (5)

To a stirring solution of compound 4 (127 mg, 0.39 mmol) in DMF (3 mL) was added compound Intermediate-9 (100 mg, 039. mmol) and DIPEA (100 mg, 0.77 mmol) at room temperature. To this added HATU (175 mg, 0.46 mmol) at room temperature and the reaction mixture was stirred at room temperature for 1 hour. the reaction mixture was diluted with water and extracted with EtOAc. Combined organic extracts were dried over anhydrous Na₂SO₄ and concentrated under reduced pressure to obtain crude product, which was purified by silica gel column chromatography eluting with 3% MeOH/DCM to afford compound 5 (150 mg, 68%). TLC: 5% MeOH/DCM (R_{f}: 0.4).

LC-MS: m/z=573.2 [M+H]⁺

A solution of compound 5 (70 mg, 0.12 mmol) in TFA (6 mL) was heated 70° C. for 1 hour then cooled and concentrated and purified by silica gel column chromatography eluting with 5% MeOH/DCM to afford compound 86-1 (20 mg, 40%) as white solid. TLC: 10% MeOH/DCM (Rf: 0.3). LC-MS: m/z=423 [M+H]⁺ (98% purity).

¹H NMR (400 MHz, DMSO-d6) δ 14.75-14-94 (m, 1H), 8.80 (d, J=13.0 Hz, 1H), 7.87 (s, 0.5H), 7.44 (s, 0.5H), 7.35-7.30 (m, 4H), 4.68-4.27 (m, 5H), 4.01-3.88 (m, 1H), 3.04 (t, J=7.1 Hz, 2H), 2.60-2.54 (m, 1H), 2.44 (d, J=9.5 Hz, 1H), 2.23 (dt, J=13.3, 6.5 Hz, 1H), 1.96 (s, 1H), 1.80-1.30 (m, 5H), 1.00-0.85 (m, 3H).

### Example 87

### (S)-1-cyclobutyl-N-(4-(methylsulfonyl)benzyl)ethanamine (2)

To a stirring solution of 1-(bromomethyl)-4-(methylsulfonyl)benzene SM1 (125 mg, 0.5 mmol) in DCM (10 ml), was added (S)-1-cyclobutylethanamine compound 1 (50 mg, 0.5 mmol), followed by DIEA (129 mg, 1 mmol) at room temperature. The reaction mixture stirred at room temperature for 16 h. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 30% EA/PE to afford compound 2 (30 mg, 22%). TLC: 30% EA/PE (R_{f}: 0.4)

LC-MS: m/z=268 [M+H]⁺

To a stirring solution of (S)-1-cyclobutyl-N-(4-(methylsulfonyl)benzyl)ethanamine compound 2 (30 mg, 0.11 mmol), in DMF (5 mL) was added (S)-2-(2,5-dioxo-2′,3′-dihydrospiro[imidazolidine-4,1′-indene]-1-yl)acetic acid intermediate 9 (28.6 mg, 0.11 mmol), followed by DIEA (28.38 mg, 0.22 mmol) and HATU (63.8 mg, 0.168 mmol), After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 30% EA/PE to afford 87-1 (10 mg, 17.8%). LC-MS: m/z=510 [M+H]⁺ at RT 1.59 (100% purity). ¹H NMR (300 MHz, DMSO-d6) δ 8.81 (d, J=10.4 Hz, 1H), 8.03-7.16 (m, 8H), 4.81-3.90 (m, 5H), 3.20 (d, J=10.8 Hz, 3H), 3.04 (t, J=7.1 Hz, 3H), 2.22 (d, J=13.4 Hz, 1H), 2.04-1.85 (m, 1H), 1.66 (dd, J=21.1, 13.9 Hz, 6H), 1.11-0.76 (m, 3H).

### Example 88

### tert-butyl 2-(5-bromo-2′,4′-dioxo-2,3-dihydrospiro[indene-1,5′-ozazolidine]-3′-yl)acetate (1)

To a stirring solution of compound SM1 (90 mg, 0.319 mmol) in DMF (5 mL) was added K₂CO₃ (88 mg, 0.638 mmol) followed by tert-butyl 2-bromoacetate (63 mg, 0.319 mmol) at room temperature and stirred at room temperature overnight. The reaction mixture was diluted with water and extracted with EtOAc. Combined organic extracts were dried over anhydrous Na₂SO₄ and concentrated under reduced pressure to obtain compound 1 (128 mg, 99%) as a yellow oil. LC-MS: m/z=340/342 [M+H-t-Bu]⁺

### 2-(5-bromo-2′,4′-dioxo-2,3-dihydrospiro[indene-1,5′-ozazolidine]-3′-yl)acetic acid (2)

To a stirring solution of compound 1 (128 mg, 0.323 mmol) in DCM (1 mL) was added TFA (1 mL) at room temperature and stirred at room temperature for 2 hours. After consumption of the starting material (by TLC), the reaction mixture was concentrated under reduced pressure to obtain crude compound 2 (120 mg, 99%) as a yellow oil. LC-MS: m/z=340/342 [M+H]⁺

To a stirring solution of compound 2 (120 mg, 0.353 mmol) in DMF (5 mL) was added 1-cyclopropyl-N-(4-fluorobenzyl)ethanamine (82 mg, 0.424 mmol) and DIPEA (137 mg, 1.059 mmol) followed by HATU (268 mg, 0.706 mmol) at room temperature and stirred at room temperature overnight. The reaction mixture was diluted with water and extracted with EtOAc. Combined organic extracts were dried over anhydrous Na₂SO₄ and concentrated under reduced pressure to obtain crude product, which was purified by prep-HPLC eluting with ACN/H₂O containing 5% TFA to afford 88-1 (50 mg, 28%) as a white solid. LC-MS: m/z=515.1/517.1 [M+H]⁺ at RT 5.740 (98.261% purity). ¹H NMR (400 MHz, DMSO-d6): δ 7.81-7.01 (m, 7H), 4.90-4.16 (m, 4H), 3.75 (d, J=7.2 Hz, 0.6H), 3.38 (d, J=7.8 Hz, 0.4H), 3.25-3.03 (m, 2H), 2.76-2.56 (m, 2H), 1.31-1.06 (m, 3H), 1.06-0.82 (m, 1H), 0.50-0.15 (m, 4H).

### Example 89

To a stirring solution of 19-1 (150 mg, 0.302 mmol) in DMF (2 mL) was added Zn(CN)₂ (106 mg, 0.905 mmol) followed by Pd(PPh₃)₄ (35 mg, 0.0302 mmol) at room temperature. The reaction mixture was stirred under microwave irradiation at 165° C. under N₂ for 30 minutes. EtOAc was added and then filtered. The filtrate was concentrated under reduced pressure to obtain crude product, which was purified by prep-HPLC eluting with ACN/H₂O containing 5% TFA to afford 89-1 (22 mg, 16%) as a white solid. LC-MS: m/z=444.2 [M+H]⁺ at RT 5.054 (92.608% purity). ¹H NMR (400 MHz, DMSO-d6): δ 8.05-7.63 (m, 3H), 7.51-7.13 (m, 5H), 4.93-4.26 (m, 4H), 3.77 (d, J=7.2 Hz, 0.6H), 3.39 (s, 0.4H), 3.20 (ddd, J=22.9, 15.0, 8.4 Hz, 2H), 2.88-2.56 (m, 2H), 1.35-1.05 (m, 3H), 0.96 (s, 1H), 0.61-0.13 (m, 4H).

### Example 90

### 3-hydroxy-2,3-dihydro-1H-inden-1-one (1)

A mixture of 2-bromobenzaldehyde SM1 (1.85 g, 0.01 mol), Pd(OAc)₂ (23 mg, 0.1 mmol) and dppf (62 mg, 0.15 mmol) was degassed three times with nitrogen, ethylene glycol (40 mL), 1-(vinyloxy)butane (3 g, 0.03 mol) and Et₃N (1.52 g, 0.015 mol) were injected sequentially. The reaction mixture was stirred at 115° C. for 16 h. After cooled, HCl (3M, 20 mL) and EtOAc (50 mL) were added. The mixture was stirred for ¹H. After separation of EtOAc phase, the aqueous layer was extracted with EtOAc (20 mL×3) and the combined organic layers were washed with brine, dried over Na₂SO₄, filtered, and concentrated under reduced pressure. The crude was purified by flash chromatography on silica gel using a mixture of (PE/EtOAc 20/80 to 50/50) as eluent to to afford compound 1 (1.2 g, 81%) as an oil. LC-MS: m/z=149 [M+H]⁺

### 3′-hydroxy-2′,3′-dihydrospiro[imidazolidine-4,1′-indene]-2,5-dione (2)

A mixture of 3-hydroxy-2,3-dihydro-1H-inden-1-one compound 1 (1 g, 6.75 mmol), (NH₄)₂CO₃ (4.24 g, 27 mmol) and KCN (0.88 g, 13.5 mmol) in EtOH/H₂O (10 mL/10 mL) was heated at 100° C. for 15 h in a sealed vial. The reaction mixture was extracted with EtOAc. Combined organic extracts were dried over anhydrous Na₂SO₄ and concentrated under reduced pressure to obtain crude product, which was used to the next step. LC-MS: m/z=219 [M+H]⁺

### spiro[imidazolidine-4,1′-indene]-2,3′,5(2′H)-trione (3)

To a stirring mixture of compound 2 (0.45 g, 2.06 mmol) in DCM (15 mL) was added Dess-Martin periodinane (1.75 g, 4.12 mmol). The mixture was stirred at room temperature for 15 h. The reaction mixture was washed with NaHCO₃ (a.q), combined organic extracts were dried over anhydrous Na₂SO₄ and concentrated under reduced pressure to obtain crude product, which was purified by silica gel column chromatography to afford compound 3 (55 mg, 15%) as an off-white solid. LC-MS: m/z=217 [M+H]⁺

To a stirring solution of compound 3 (30 mg, 0.14 mmol) in DMF (1 mL) was added intermediate-23 (41 mg, 0.14 mmol) followed by K₂CO₃ (29 mg, 0.21 mmol) at room temperature. The reaction mixture was stirred for 1.5 hours at room temperature. The reaction mixture was diluted with water and extracted with EtOAc. Combined organic extracts were dried over anhydrous Na2SO4 and concentrated under reduced pressure. The residue was purified by Prep-HPLC to afford compound 90-1 (18 mg, 30%) as an off-white solid. LC-MS: m/z=432 [M+H]⁺ at RT 1.54 (100% purity). ¹H NMR (400 MHz, DMSO-d6): δ 8.94 (d, J=16.1 Hz, 1H), 7.93-7.59 (m, 4H), 7.50-7.15 (m, 4H), 4.76 (s, 1H), 4.71-4.45 (m, 2H), 4.41-4.17 (m, 2H), 3.74 (d, J=7.3 Hz, 1H), 3.38 (d, J=8.3 Hz, 1H), 3.11 (dd, J=18.7, 9.9 Hz, 1H), 3.00-2.87 (m, 1H), 1.16 (dt, J=13.9, 12.3 Hz, 3H), 0.94 (s, 1H), 0.55-0.07 (m, 4H).

### Example 91

### N-(2,5-dioxo-2′,3′-dihydrospiro[imidazolidine-4,1′-indene]-5′-yl)acetamide (1)

To a stirring solution of SM1 (1.9 g, 10 mmol) in EtOH (15 mL), H₂O (15 mL) was added (NH₄)₂CO₃ (4.8 g, 50 mmol) followed by potassium cyanide (1.3 g, 20 mmol) at room temperature. The reaction mixture was heated to 80° C. for 72 hours. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 1 (0.9 g, 35%) as a yellow solid. LC-MS: m/z=260 [M+H]⁺

### tert-Butyl 2-(5′-acetamido-2,5-dioxo-2′,3′-dihydrospiro[imidazolidine-4,1′-indene]-1-yl) acetate (2)

To a stirring solution of compound 1 (655 mg, 2.53 mmol) in DMF (5 mL) was added K₂CO₃ (524.4 mg, 3.8 mmol) followed by tert-butyl 2-bromoacetate (518.7 mg, 2.66 mmol) at room temperature and stirred at room temperature for 1 hour. The reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 2 (616 mg, 65%) as a yellow solid. LC-MS: m/z=375 [M+H]⁺

### 2-(5′-Acetamido-2,5-dioxo-2′,3′-dihydrospiro[imidazolidine-4,1′-indene]-1-yl)acetic acid (3)

To a stirring solution of compound 2 (616 mg, 1.65 mmol) in DCM (3 mL) was added TFA (3 mL) at room temperature and stirred at room temperature for 2 hours. After consumption of the starting material (by TLC), the reaction mixture was concentrated under reduced pressure to obtain crude compound 3 (500 mg, 96%) as a yellow solid. LC-MS: m/z=318 [M+H]⁺

### 2-(5′-Acetamido-2,5-dioxo-2′,3′-dihydrospiro[imidazolidine-4,1′-indene]-1-yl)-N-benzyl-N—((S)-1-cyclopropylethyl)acetamide (4)

To a stirring solution of compound 3 (106.8 mg, 0.337 mmol) in DMF (1 mL) was added Intermediate 24 (59 mg, 0.337 mmol) and DIPEA (96 mg, 0.741 mmol) followed by HATU (141 mg, 0.371 mmol) at room temperature and stirred at room temperature for 1 hour. The reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 4 (150 mg, 94%) as a yellow solid. LC-MS: m/z=475 [M+H]⁺

### 2-(5′-Amino-2,5-dioxo-2′,3′-dihydrospiro[imidazolidine-4,1′-indene]-1-yl)-N-benzyl-N—((S)-1-cyclopropylethyl)acetamide (5)

To a stirring solution of compound 4 (150 mg, 0.316 mmol) in MeOH (4 mL) was added con. HCl (1 mL) followed by H₂O (1 mL) at room temperature. The reaction mixture was heated to 80° C. for 1 hour. After consumption of the starting material (by TLC), the resulting mixture was cooled to room temperature, neutralized by 4M NaOH solution and the precipitate was filtered and dried to afford compound 5 (85 mg, 62%) as an yellow solid. LC-MS: m/z=433 [M+H]⁺

To a stirring solution of compound 5 (40 mg, 0.0926 mmol) in DMF (1 mL) was added DIPEA (14.3 mg, 0.111 mmol) and 2-cyanoacetic acid (7.9 mg, 0.0926 mmol) followed by HATU (38.8 mg, 0.102 mmol) at room temperature and stirred at room temperature for 1 hour. The reaction mixture was diluted with water and extracted with EtOAc. Combined organic extracts were dried over anhydrous Na2SO4 and concentrated under reduced pressure to obtain crude product, which was purified by Prep-TLC eluting with 5% MeOH/DCM to afford 91-1 (22 mg, 48%) as a white solid. LC-MS: m/z=500.2 [M+H]⁺. ¹H NMR (300 MHz, MeOD): δ 7.63 (s, 1H), 7.50-7.11 (m, 7H), 4.76 (d, J=12.9 Hz, 2H), 4.43 (dd, J=26.9, 18.9 Hz, 2H), 3.81 (s, 1H), 3.12 (s, 2H), 2.72 (dd, J=13.4, 5.5 Hz, 2H), 2.33 (dd, J=14.2, 7.1 Hz, 2H), 1.32-1.20 (m, 3H), 0.93 (s, 1H), 0.56 (s, 1H), 0.27 (s, 3H).

### Example 92

### N-(2,5-dioxo-2′,3′-dihydrospiro[imidazolidine-4,1′-indene]-5′-yl)acetamide (1)

To a stirring solution of SM1 (1.9 g, 10 mmol) in EtOH (15 mL), H₂O (15 mL) was added (NH₄)₂CO₃ (4.8 g, 50 mmol) followed by potassium cyanide (1.3 g, 20 mmol) at room temperature. The reaction mixture was heated to 80° C. for 72 hours. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 1 (0.9 g, 35%) as a yellow solid. LC-MS: m/z 260 [M+H]⁺

### tert-Butyl 2-(5′-acetamido-2,5-dioxo-2′,3′-dihydrospiro[imidazolidine-4,1′-indene]-1-yl) acetate (2)

To a stirring solution of compound 1 (655 mg, 2.53 mmol) in DMF (5 mL) was added K₂CO₃ (524.4 mg, 3.8 mmol) followed by tert-butyl 2-bromoacetate (518.7 mg, 2.66 mmol) at room temperature and stirred at room temperature for 1 hour. The reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 2 (616 mg, 65%) as a yellow solid. LC-MS: m/z 375 [M+H]⁺

### 2-(5′-Acetamido-2,5-dioxo-2′,3′-dihydrospiro[imidazolidine-4,1′-indene]-1-yl)acetic acid (3)

To a stirring solution of compound 2 (616 mg, 1.65 mmol) in DCM (3 mL) was added TFA (3 mL) at room temperature and stirred at room temperature for 2 hours. After consumption of the starting material (by TLC), the reaction mixture was concentrated under reduced pressure to obtain crude compound 3 (500 mg, 96%) as a yellow solid. LC-MS: m/z 318 [M+H]⁺

### 2-(5′-Acetamido-2,5-dioxo-2′,3′-dihydrospiro[imidazolidine-4,1′-indene]-1-yl)-N-benzyl-N—((S)-1-cyclopropylethyl)acetamide (4)

To a stirring solution of compound 3 (106.8 mg, 0.337 mmol) in DMF (1 mL) was added Intermediate 24 (59 mg, 0.337 mmol) and DIPEA (96 mg, 0.741 mmol) followed by HATU (141 mg, 0.371 mmol) at room temperature and stirred at room temperature for 1 hour. The reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 4 (150 mg, 94%) as a yellow solid. LC-MS: m/z=475 [M+H]⁺

### 2-(5′-Amino-2,5-dioxo-2′,3′-dihydrospiro[imidazolidine-4,1′-indene]-1-yl)-N-benzyl-N—((S)-1-cyclopropylethyl)acetamide (5)

To a stirring solution of compound 4 (150 mg, 0.316 mmol) in MeOH (4 mL) was added con. HCl (1 mL) followed by H₂O (1 mL) at room temperature. The reaction mixture was heated to 80° C. for 1 hour. After consumption of the starting material (by TLC), the resulting mixture was cooled to room temperature, neutralized by 4M NaOH solution and the precipitate was filtered and dried to afford compound 5 (85 mg, 62%) as an yellow solid. LC-MS: m/z=433 [M+H]⁺

To a stirring solution of compound 5 (39 mg, 0.0903 mmol) in DCM (3 mL) was added triphosgen (10.7 mg, 0.03612 mmol) followed by Et₃N (10.9 mg, 0.1084 mmol) at 0° C. and the reaction mixture was stirred at room temperature for 2 hours. Then MeOH (3 mL) was added at room temperature and the resulting mixture was stirred at room temperature for 2 hours and concentrated under reduced pressure to obtain crude product, which was purified by Prep-TLC eluting with 10% MeOH/DCM to afford 92-1 (20 mg, 45%) as a white solid. LC-MS: m/z=491.0 [M+H]⁺. ¹H NMR (400 MHz, DMSO-d6): δ 9.72 (s, 1H′), 8.73 (d, J=16.6 Hz, 1H), 7.52-7.07 (m, 8H), 4.74 (s, 1H), 4.62 (s, 1H), 4.47-4.15 (m, 2H), 3.69 (d, J=16.0 Hz, 3H), 3.38 (s, 1H), 2.99 (dd, J=13.0, 6.6 Hz, 2H), 2.54 (d, J=7.0 Hz, 1H), 2.28-2.11 (m, 1H), 1.21-1.06 (m, 3H), 0.94 (s, 1H), 0.56-0.12 (m, 4H).

### Example 93

### N-(2,3-dihydro-1H-inden-4-yl)acetamide (1)

To a stirring solution of acetic anhydride (14 mL, 278 mmol) in EtOH (150 ml) was added the solution of 2,3-dihydro-1H-inden-4-amine SM (10 g, 75 mmol) in EtOH (50 mL) at 0° C. and stirred at room temperature for 3 hours. After consumption of the starting material (by TLC), the solvent from reaction mixture was removed under reduced pressure to afford compound 1 (13.1 g, 100%) as an off-white solid used for next step directly. LC-MS: m/z=176.2 [M+H]⁺

### N-(3-oxo-2,3-dihydro-1H-inden-4-yl)acetamide (2)

To a stirring solution of compound 1 (13.1 g, 75 mmol) in acetone (400 mL) and 15% aqueous MgSO₄ (12 g in 65 mL water) was added KMnO₄ (28 g, 176 mmol) at 0° C. and stirred at room temperature for 4 h. After consumption of the starting material (by TLC), quenched the reaction with Na₂SO₃ aqueous. The mixture was filtered through Celite and the filtrate was concentrate under reduced pressure. The residue was diluted with water and extracted with DCM. Combined organic extracts were dried over anhydrous Na₂SO₄ and concentrated under reduced pressure to obtain compound 2 (12.4 g, 87%) as an light yellow solid used for next step directly. LC-MS: m/z=190.2 [M+H]⁺

### 7-amino-2,3-dihydro-1H-inden-1-one (3)

A mixture of compound 2 (12.4 g, 0.065 mol) in 6N HCl (150 mL) was heated to 90° C. for 3 hours. After consumption of the starting material (by TLC), the mixture was cooled to room temperature and NaCO3 was added in small portions followed by addition of 2M NaOH until the mixture at pH 8. The aqueous layer was extracted with EtOAc. Combined organic extracts were dried over anhydrous Na₂SO₄ and concentrated under reduced pressure to afford compound 3 (9.4 g, 98%) as a brown solid used for next step directly. LC-MS: m/z=148.1 [M+H]⁺

### 7-fluoro-2,3-dihydro-1H-inden-1-one (4)

To a stirring solution of NOBF₄ (2.16 g, 18.5 mmol) in acetone (30 mL) was added compound 3 (2.34 g, 15.9 mmol) dissolved in acetone (30 mL) at −15° C. After 30 min, more NOBF₄ (0.9 g, 7.7 mmol) was added to the mixture. After consumption of the starting material (by TLC), the reaction mixture was poured into DCM and stirred for 30 min. The solvent was removed under reduced pressure, the residue was added toluene (50 mL) and heated to 85° C. for 4 hours. The mixture was cooled to room temperature and concentrated under reduced pressure to obtain crude product, which was purified by silica gel column chromatography eluting with 10% EtOAc/PE to afford compound 4 (0.23 g, 10%). LC-MS: m/z=151.1 [M+H]⁺

### 7′-fluoro-2′,3′-dihydrospiro[imidazolidine-4,1′-indene]-2,5-dione (5)

To a stirring solution of compound 4 (0.23 g, 1.53 mmol) in 50% EtOH/H₂O (20 mL) was added (NH₄)₂CO₃ (1.8 g, 18.3 mmol) followed by potassium cyanide (0.3 g, 4.59 mmol) at room temperature. The reaction mixture was heated to 70° C. for 24 h. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 10% MeOH/DCM to afford compound 5 (0.21 g, 62%). LC-MS: m/z=221.0 [M+H]⁺

To a stirring solution of compound 5 (26 mg, 0.12 mmol) and compound 6 (35 mg, 0.12 mmol) in DMF (1 mL) was added K₂CO₃ (36 mg, 0.24 mmol) and stirred at room temperature for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by prep-hplc to afford compound 93-1 (20 mg, 38%) as an white solid. LC-MS: m/z=436.2 [M+H]⁺ at RT 4.62 (98.77% purity). ¹H NMR (300 MHz, DMSO-d6) δ 8.89 (d, J=10.2 Hz, 1H), 7.51-6.81 (m, 8H), 4.74 (s, 1H), 4.59 (d, J=8.2 Hz, 1H), 4.50-4.25 (m, 1H), 4.20 (s, 1H), 3.72-3.64 (m, 0.5H), 3.42-3.37 (m, 0.5H), 3.14-2.98 (m, 2H), 2.58 (dd, J=13.0, 6.9 Hz, 1H), 2.31-2.19 (m, 1H), 1.22-1.04 (m, 3H), 0.99-0.90 (m, 1H), 0.53-0.10 (m, 4H).

### Example 94

### (S)-1-cyclobutylethanamine hydrogen chloride (1)

To a solution of intermediate 16 (0.4 g, 2 mmol) was added HCl (5 ml, 2N in dioxane), the reaction mixture was stirred at room temperature for 2 hours. Concentrated under reduced pressure to obtain crude compound 1 (0.3 g, 111%) as white solid. TLC: 50% EtOAc/hexane (R_{f}: 0.1)

### (1S)-1-cyclobutyl-N-(1-phenylethyl)ethanamine (3)

To a solution of (S)-1-cyclobutylethanamine hydrogen chloride 1 (0.2 g, 1.5 mmol) and (1-bromoethyl)benzene 2 (0.55 g, 3 mmol) in MeCN (20 mL) was added K₂CO₃ (0.82 g, 6 mmol), the reaction mixture was stirred at 80° C. for 16 h. Cooled to room temperature and concentrated under reduced pressure to obtain crude product, which was purified by silica gel column chromatography (EtOAc/Hexane 1:4) to afford (1S)-1-cyclobutyl-N-(1-phenylethyl)ethanamine 3 (0.13 g, 43%) as yellow oil. ¹H NMR (300 MHz, CDCl₃) δ 7.32 (td, J=12.4, 5.8 Hz, 4H), 3.88 (dd, J=13.0, 6.5 Hz, 1H′), 2.68-2.49 (m, 1H), 2.24 (dd, J=13.2, 7.5 Hz, 1H), 1.97-1.47 (m, 6H), 1.37 (d, J=4.2 Hz, 3H), 0.89 (dd, J=10.7, 6.1 Hz, 3H).

To a mixture of (1S)-1-cyclobutyl-N-(1-phenylethyl)ethanamine 3 (41 mg, 0.2 mmol) and intermediate 9 (52 mg, 0.2 mmol) in DCM (10 mL) was added TEA (41 mg, 0.4 mmol) and 2,4,6-tripropyl-1,3,5,2,4,6-trioxatriphosphorinane-2,4,6-trioxide (254 mg, 0.4 mmol) at 0° C. under N₂, then stirred at room temperature for 2 hours. After consumption of the starting material (by TLC), diluted with water and extracted with DCM. Combined organic extracts were dried over anhydrous MgSO₄ and concentrated under reduced pressure to obtain crude product, which was purified by PREP-HPLC (MeCN/H₂O 3:2) to afford 94-1 (25 mg, 28%) as white solid. LC-MS: m/z=446.2 [M+H]⁺ at RT 5.14 (98.58% purity). ¹H NMR (300 MHz, CD₃OD) δ 7.71-7.07 (m, 9H), 5.19 (d, J=6.3 Hz, 1H), 4.61 (d, J=15.6 Hz, 3H), 3.12 (s, 2H), 2.92 (d, J=8.4 Hz, 1H), 2.81-2.66 (m, 1H), 2.33 (d, J=5.7 Hz, 2H), 1.95 (s, 2H), 1.66 (t, J=33.0 Hz, 6H), 1.36-1.18 (m, 3H).

### Example 95

### N-(2,3-dihydro-1H-inden-4-yl)acetamide (1)

To a stirring solution of acetic anhydride (14 ml, 278 mmol) in EtOH (150 ml) was added the solution of 2,3-dihydro-1H-inden-4-amine SM (10 g, 75 mmol) in EtOH (50 mL) at 0° C. and stirred at room temperature for 3 hours. After consumption of the starting material (by TLC), the solvent from reaction mixture was removed under reduced pressure to afford compound 1 (13.1 g, 100%) as an off-white solid used for next step directly. LC-MS: m/z=176.2 [M+H]⁺

### N-(3-oxo-2,3-dihydro-1H-inden-4-yl)acetamide (2)

To a stirring solution of compound 1 (13.1 g, 75 mmol) in acetone (400 mL) and 15% aqueous MgSO₄ (12 g in 65 ml water) was added KMnO₄ (28 g, 176 mmol) at 0° C. and stirred at room temperature for 4 h. After consumption of the starting material (by TLC), quenched the reaction with Na₂SO₃ aqueous. The mixture was filtered through Celite and the filtrate was concentrate under reduced pressure. The residue was diluted with water and extracted with DCM. Combined organic extracts were dried over anhydrous Na₂SO₄ and concentrated under reduced pressure to obtain compound 2 (12.4 g, 87%) as an light yellow solid used for next step directly. LC-MS: m/z=190.2 [M+H]⁺

### 7-amino-2,3-dihydro-1H-inden-1-one (3)

A mixture of compound 2 (12.4 g, 0.065 mol) in 6N HCl (150 mL) was heated to 90° C. for 3 hours. After consumption of the starting material (by TLC), the mixture was cooled to room temperature and Na₂CO₃ was added in small portions followed by addition of 2M NaOH until the mixture at pH 8. The aqueous layer was extracted with EtOAc. Combined organic extracts were dried over anhydrous Na₂SO₄ and concentrated under reduced pressure to afford compound 3 (9.4 g, 98%) as a brown solid used for next step directly. LC-MS: m/z=148.1 [M+H]⁺

### 7-bromo-2,3-dihydro-1H-inden-1-one (4)

To a stirring solution of compound 3 (1.0 g, 6.8 mmol) in 48% HBr_{aq} (2 mL) and EtOH (8 mL) was added NaNO₂ (0.62 g, 8.6 mmol) dissolved in H₂O (1 mL) at 0° C. and kept at 0° C. for 15 min. Then a solution of CuBr (0.54 g, 3.6 mmol) in 48% HBr_{aq} (8 mL) was added to the mixture at 95° C. and kept at 95° C. for 15 min. After consumption of the starting material (by TLC), the reaction mixture was diluted with water and extracted with EtOAc. The organic layer was washed with saturated NaHCO₃ aq. Combined organic extracts were dried over anhydrous Na₂SO₄ and concentrated under reduced pressure to obtain crude product, which was purified by silica gel column chromatography eluting with 10% EtOAc/PE to afford compound 4 (0.54 g, 37%) as an off-white solid. LC-MS: m/z=212.0 [M+H]⁺

### 3-oxo-2,3-dihydro-1H-indene-4-carbonitrile (5)

To a stirring solution of compound 4 (0.54 g, 2.56 mmol) in DMF (2.5 mL) was added Zn(CN)₂ (751 mg, 6.4 mmol) followed by Pd(PPh₃)₄ (88 mg, 0.08 mmol) at room temperature. The reaction mixture was heated to 165° C. by microwave for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 10% EtOAc/PE to afford compound 5 (0.3 g, 74%) as a yellow solid. LC-MS: m/z=158.1 [M+H]⁺

### 2,5-dioxo-2′,3′-dihydrospiro[imidazolidine-4,1′-indene]-7′-carbonitrile (6)

To a stirring solution of compound 5 (0.3 g, 1.91 mmol) in 50% EtOH/H₂O (20 mL) was added (NH₄)₂CO₃ (2.2 g, 22.9 mmol) followed by potassium cyanide (0.8 g, 12.28 mmol) at room temperature. The reaction mixture was heated to 75° C. for 18 h. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 10% MeOH/DCM to afford compound 6 (0.22 g, 50%). LC-MS: m/z=228.2 [M+H]⁺

To a stirring solution of compound 6 (27 mg, 0.12 mmol) and intermediate-23 (35 mg, 0.12 mmol) in DMF (1 mL) was added K₂CO₃ (33 mg, 0.24 mmol) and stirred at room temperature for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by prep-hplc to afford compound 95-1 (7 mg, 13%) as an off-white solid. LC-MS: m/z=443.2 [M+H]⁺ at RT 4.43 (99.14% purity). ¹H NMR (300 MHz, CD₃OD-d6) δ 7.75-7.25 (m, 8H), 4.89-4.66 (m, 2H), 4.62-4.33 (m, 2H), 3.82-3.76 (s, 0.5H), 3.41-3.33 (m, 0.5H), 3.25-3.05 (m, 2H), 2.91-2.78 (m, 1H), 2.52-2.39 (m, 1H), 1.42-1.10 (m, 3H), 1.02-0.94 (m, 1H), 0.64-0.13 (m, 4H).

### Example 96

### (S)-2-(5-acetamido-2′,4′-dioxo-2,3-dihydrospiro[indene-1,5′-ozazolidine]-3′-yl)-N-benzyl-N-(1-cyclopropylethyl)acetamide

A solution of intermediate 8 (1 g, 3.85 mmol), intermediate 23 (1.14 g, 3.85 mmol) and K₂CO₃ (1 g, 7.7 mmol) in DMF (20 mL) was stirred for 2 hours. After consumption of the starting material (by TLC), the reaction mixture was diluted with sat NaCl aqueous and extracted with EtOAc. Combined organic extracts were dried over anhydrous Na₂SO₄ and concentrated under reduced pressure to obtain crude product, which was purified by silica gel column chromatography to afford 96-1 (1.35 g, 73%) as a yellow liquid. TLC: 35% PE/EA. ¹H NMR (400 MHz, DMSO-d₆): δ 10.12 (s, 1H), 7.76 (d, J=5.2 Hz, 1H), 7.41-7.26 (m, 7H), 5.09-4.11 (m, 5H), 3.78 (d, J=6.6 Hz, 1H), 3.20-2.99 (m, 2H), 2.65 (d, J=6.8 Hz, 1H), 2.07 (s, 3H), 1.24-1.11 (m, 3H), 0.97 (s, 1H), 0.50-0.23 (m, 4H). LC-MS: m/z=476.1 [M+H]⁺ (96.32% purity, 214 nm)

### Example 97

To a stirring solution of 89-1 (60 mg, 0.135 mmol) in DMSO (0.5 mL) was added K₂CO₃ (33 mg, 0.237 mmol) followed by H₂O₂ (30%, 0.2 mL) at 0-5° C. The reaction mixture was stirred at room temperature for 5 minutes. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by prep-HPLC eluting with ACN/H₂O containing 5% TFA to afford 97-1 (50 mg, 80%) as a white solid. LC-MS: m/z=462.2 [M+H] at RT 4.567 (98.040% purity). ¹H NMR (400 MHz, DMSO): δ 8.04 (s, 1H), 7.89 (s, 1H), 7.81 (t, J=8.6 Hz, 1H), 7.62-6.92 (m, 7H), 4.91-4.24 (m, 4H), 3.78 (dd, J=16.1, 6.8 Hz, 0.6H), 3.39 (d, J=7.5 Hz, 0.4H), 3.30-3.00 (m, 2H), 2.81-2.53 (m, 2H), 1.18 (dd, J=26.3, 6.6 Hz, 3H), 0.98 (s, 1H), 0.60-0.10 (m, 4H).

### Example 98

### N-(2,5-dioxo-2′,3′-dihydrospiro[imidazolidine-4,1′-indene]-5′-yl)acetamide (1)

To a stirring solution of SM1 (1.9 g, 10 mmol) in EtOH (15 mL), H₂O (15 mL) was added (NH₄)₂CO₃ (4.8 g, 50 mmol) followed by potassium cyanide (1.3 g, 20 mmol) at room temperature. The reaction mixture was heated to 80° C. for 72 hours. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 1 (0.9 g, 35%) as a yellow solid. LC-MS: m/z 260 [M+H]⁺

### tert-Butyl 2-(5′-acetamido-2,5-dioxo-2′,3′-dihydrospiro[imidazolidine-4,1′-indene]-1-yl) acetate (2)

To a stirring solution of compound 1 (655 mg, 2.53 mmol) in DMF (5 mL) was added K₂CO₃ (524.4 mg, 3.8 mmol) followed by tert-butyl 2-bromoacetate (518.7 mg, 2.66 mmol) at room temperature and stirred at room temperature for 1 hour. The reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 2 (616 mg, 65%) as a yellow solid. LC-MS: m/z=375 [M+H]⁺

### 2-(5′-Acetamido-2,5-dioxo-2′,3′-dihydrospiro[imidazolidine-4,1′-indene]-1-yl)acetic acid (3)

To a stirring solution of compound 2 (616 mg, 1.65 mmol) in DCM (3 mL) was added TFA (3 mL) at room temperature and stirred at room temperature for 2 hours. After consumption of the starting material (by TLC), the reaction mixture was concentrated under reduced pressure to obtain crude compound 3 (500 mg, 96%) as a yellow solid. LC-MS: m/z=318 [M+H]⁺

### 2-(5′-Acetamido-2,5-dioxo-2′,3′-dihydrospiro[imidazolidine-4,1′-indene]-1-yl)-N-benzyl-N—((S)-1-cyclopropylethyl)acetamide (4)

To a stirring solution of compound 3 (106.8 mg, 0.337 mmol) in DMF (1 mL) was added Intermediate 24 (59 mg, 0.337 mmol) and DIPEA (96 mg, 0.741 mmol) followed by HATU (141 mg, 0.371 mmol) at room temperature and stirred at room temperature for 1 hour. The reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 4 (150 mg, 94%) as a yellow solid. LC-MS: m/z=475 [M+H]⁺

### 2-(5′-Amino-2,5-dioxo-2′,3′-dihydrospiro[imidazolidine-4,1′-indene]-1-yl)-N-benzyl-N—((S)-1-cyclopropylethyl)acetamide (5)

To a stirring solution of compound 4 (150 mg, 0.316 mmol) in MeOH (4 mL) was added con. HCl (1 mL) followed by H₂O (1 mL) at room temperature. The reaction mixture was heated to 80° C. for 1 hour. After consumption of the starting material (by TLC), the resulting mixture was cooled to room temperature, neutralized by 4M NaOH solution and the precipitate was filtered and dried to afford compound 5 (85 mg, 62%) as an yellow solid. LC-MS: m/z=433 [M+H]⁺

To a stirring solution of compound 5 (39 mg, 0.0903 mmol) in DCM (3 mL) was added triphosgen (10.7 mg, 0.03612 mmol) followed by Et₃N (10.9 mg, 0.1084 mmol) at 0° C. and the reaction mixture was stirred at room temperature for 3 hours. Then 2 M methanamine in THF (0.05 mL, 0.1 mmol) was added at room temperature and the resulting mixture was stirred at room temperature for ¹H and concentrated under reduced pressure to obtain crude product, which was purified by Prep-TLC eluting with 10% MeOH/DCM to afford 98-1 (5 mg, 11%) as a white solid. LC-MS: m/z=490.2 [M+H]⁺. ¹H NMR (400 MHz, DMSO-d6): δ 8.69 (d, J=16.7 Hz, 1H), 8.56 (s, 1H), 7.50-7.00 (m, 8H), 6.01 (s, 1H), 4.73 (s, 1H), 4.62 (s, 1H), 4.50-4.15 (m, 2H), 3.71 (s, 1H), 3.36 (s, 1H), 3.02-2.91 (m, 2H), 2.65-2.52 (m, 3H), 2.18 (dd, J=20.4, 10.5 Hz, 1H), 1.20-1.06 (m, 3H), 0.93 (s, 1H), 0.50-0.30 (m, 2H), 0.29-0.14 (m, 2H).

### Example 99

### N-(2,5-dioxo-2′,3′-dihydrospiro[imidazolidine-4,1′-indene]-5′-yl)acetamide (1)

To a stirring solution of SM1 (1.9 g, 10 mmol) in EtOH (15 mL), H₂O (15 mL) was added (NH₄)₂CO₃ (4.8 g, 50 mmol) followed by potassium cyanide (1.3 g, 20 mmol) at room temperature. The reaction mixture was heated to 80° C. for 72 hours. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 1 (0.9 g, 35%) as a yellow solid. LC-MS: m/z 260 [M+H]⁺

### tert-Butyl 2-(5′-acetamido-2,5-dioxo-2′,3′-dihydrospiro[imidazolidine-4,1′-indene]-1-yl) acetate (2)

To a stirring solution of compound 1 (655 mg, 2.53 mmol) in DMF (5 mL) was added K₂CO₃ (524.4 mg, 3.8 mmol) followed by tert-butyl 2-bromoacetate (518.7 mg, 2.66 mmol) at room temperature and stirred at room temperature for 1 hour. The reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 2 (616 mg, 65%) as a yellow solid. LC-MS: m/z 375 [M+H]⁺

### 2-(5′-Acetamido-2,5-dioxo-2′,3′-dihydrospiro[imidazolidine-4,1′-indene]-1-yl)acetic acid (3)

To a stirring solution of compound 2 (616 mg, 1.65 mmol) in DCM (3 mL) was added TFA (3 mL) at room temperature and stirred at room temperature for 2 hours. After consumption of the starting material (by TLC), the reaction mixture was concentrated under reduced pressure to obtain crude compound 3 (500 mg, 96%) as a yellow solid. LC-MS: m/z 318 [M+H]⁺

### 2-(5′-Acetamido-2,5-dioxo-2′,3′-dihydrospiro[imidazolidine-4,1′-indene]-1-yl)-N-benzyl-N—((S)-1-cyclopropylethyl)acetamide (4)

To a stirring solution of compound 3 (106.8 mg, 0.337 mmol) in DMF (1 mL) was added Intermediate 24 (59 mg, 0.337 mmol) and DIPEA (96 mg, 0.741 mmol) followed by HATU (141 mg, 0.371 mmol) at room temperature and stirred at room temperature for 1 hour. The reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 4 (150 mg, 94%) as a yellow solid. LC-MS: m/z=475 [M+H]⁺

### 2-(5′-Amino-2,5-dioxo-2′,3′-dihydrospiro[imidazolidine-4,1′-indene]-1-yl)-N-benzyl-N—((S)-1-cyclopropylethyl)acetamide (5)

To a stirring solution of compound 4 (150 mg, 0.316 mmol) in MeOH (4 mL) was added con. HCl (1 mL) followed by H₂O (1 mL) at room temperature. The reaction mixture was heated to 80° C. for 1 hour. After consumption of the starting material (by TLC), the resulting mixture was cooled to room temperature, neutralized by 4M NaOH solution and the precipitate was filtered and dried to afford compound 5 (85 mg, 62%) as an yellow solid. LC-MS: m/z=433 [M+H]⁺

To a stirring solution of compound 5 (45 mg, 0.142 mmol) in DCM (2 mL) was added triphosgene (46.4 mg, 0.156 mmol) followed by Et₃N (86.1 mg, 0.852 mmol) at 0° C. and the reaction mixture was stirred at room temperature for 0.5 h. Then methylamine hydrochloride (10.4 mg, 0.156 mmol) was added at room temperature and the resulting mixture was stirred at room temperature for 0.5 h and concentrated under reduced pressure to obtain crude product, which was purified by Prep-HPLC to afford 99-1 (20 mg, 26%) as a white solid. LC-MS: m/z=547.2 [M+H]⁺. ¹H NMR (400 MHz, DMSO-d6): δ 8.54 (s, 1H), 7.91 (d, J=4.6 Hz, 1H), 7.54-6.89 (m, 8H), 6.02 (s, 1H), 4.84-4.61 (m, 2H), 4.50-4.25 (m, 2H), 3.74 (dd, J=15.5, 8.4 Hz, 1H), 3.41 (d, J=20.4 Hz, 2H), 3.24-3.14 (m, 1H), 2.97 (s, 1H), 2.69-2.55 (m, 6H), 1.17 (dd, J=33.0, 6.6 Hz, 3H), 1.00 (t, J=7.0 Hz, 1H), 0.55-0.12 (m, 4H).

### Example 100

### N-benzyl-2-(5-bromo-2′,4′-dioxo-2,3-dihydrospiro[indene-1,5′-ozazolidine]-3′-yl)-N-(1-cyclopropylethyl)acetamide(1)

To a solution of Intermediate 25 (200 mg, 0.71 mmol) was added Intermediate 13 (231 mg, 0.78 mmol) and K₂CO₃ (196 mg, 1.42 mmol) in DMF (4 mL). The resulting mixture was stirred at room temperature for 2 hours. Quenched the reaction with saturated aqueous solution of NH₄Cl, extracted with ethyl acetate(20 mL×3). The organic layer was combined, concentrated and purified by chromatography to give compound 1 (300 mg, 0.60 mmol) as solid. TLC: 50% EtOAc/Petroleum ether (Rf: 0.5). LC-MS: m/z=497/499 [M+H]⁺

### N-benzyl-N-(1-cyclopropylethyl)-2-(2′,4′-dioxo-5-(4,4,5,5-tetramethyl-1,3,2-dioxaborolan-2-yl)-2,3-dihydrospiro[indene-1,5′-ozazolidine]-3′-yl)acetamide(2)

To a solution of compound 1 (200 mg, 0.40 mmol) in dioxane(5 mL) was added Pd₂(dba)₃ (18.3 mg, 0.02 mmol), PCy₃ (22.4 mg, 0.08 mmol), AcOK(78.5 mg, 0.02 mmol) and B(pin)₂ (507.8 mg, 2.0 mmol). The resulting mixture was stirred at reflux overnight. The reaction was quenched by saturated aqueous solution of NH₄Cl. Extracted by ethyl acetate(20 mL×3). The organic layer was combined, concentrated and purified by chromatography to give compound 2 (50 mg, 0.09 mmol). TLC: 50% EtOAc/Petroleum ether (Rf: 0.5). LC-MS: m/z=545 [M+H]⁺

### N-benzyl-N-(1-cyclopropylethyl)-2-(5-hydroxy-2′,4′-dioxo-2,3-dihydrospiro[indene-1,5′-ozazolidine]-3′-yl)acetamide(3)

To s solution of compound 2 (50 mg, 0.09 mmol) in dioxane(2 mL) was added H₂O₂ (1 mL, 30%). The resulting solution was stirred at room temperature for 1 hour. Evaporate the solvent to give crude compound 3 (20 mg, 80% purity). Use as it without further purification. TLC: 50% EtOAc/Petroleum ether (Rf: 0.4). LC-MS: m/z=535 [M+H]⁺

To a solution of crude compound 3 (20 mg) in DMF (2 mL) was added MeI (9.8 mg, 0.07 mmol) and K₂CO₃ (14.3 mg, 0.1 mmol). The resulting mixture was stirred at room temperature for 2 hours. TLC showed the completion of the reaction. Purified by prep-HPLC (MeCN in H₂O (0.1% TFA) from 30%-70%) to give 100-1 (5 mg, 0.01 mmol, 24%) as white solid. LC-MS: m/z=449.1 [M+H]⁺ at RT 5.26 (95.76% purity). ¹H NMR (301 MHz, CDCl₃) δ 7.45-7.33 (m, 2H), 7.34-7.14 (m, 4H), 6.83 (m, 2H), 4.84-4.59 (m, 2H), 4.46 (m, 1H), 4.27 (m, 1H), 4.01 (m, 1H), 3.92-3.58 (m, 3H), 3.31-2.98 (m, 2H), 2.82 (m, 1H), 2.65-2.45 (m, 1H), 1.34-1.12 (m, 3H), 0.95-0.74 (m, 2H), 0.57 (m, 1H), 0.45-0.19 (m, 3H).

### Example 101

### 1-(bromomethyl)-4-ethynylbenzene (2)

To a solution of (4-ethynylphenyl)methanol 1 (0.52 g, 4 mmol) in DCM (20 mL) was added PBr₃ (1.08 g, 4 mmol) at 0° C. under N₂, the reaction mixture was stirred at room temperature for 1 hour. After consumption of the starting material (by TLC), diluted with water and extracted with DCM. Combined organic extracts were dried over anhydrous MgSO₄ and concentrated under reduced pressure to obtain crude product, which was purified by silica gel column chromatography (EtOAc/Hexane 1:6) to afford compound 2 (0.3 g, 68%) as yellow oil. ¹H NMR (300 MHz, CDCl₃) δ 7.56 (d, J=8.4 Hz, 2H), 7.37 (d, J=8.2 Hz, 2H), 4.49 (s, 2H), 4.47 (s, 1H).

### (S)-1-cyclobutyl-N-(4-ethynylbenzyl)ethanamine (4)

To a solution of 1-(bromomethyl)-4-ethynylbenzene 2 (0.2 g, 1.5 mmol) and (S)-1-cyclobutylethanamine hydrogen chloride 3 (0.135 g, 1 mmol) in MeCN (20 mL) was added K₂CO₃ (0.53 g, 4 mmol), the reaction mixture was stirred at 80° C. for 16 h. Cooled to room temperature and concentrated under reduced pressure to obtain crude product, which was purified by combiflash (EtOAc/Hexane 1:4) to afford compound 4 (0.13 g, 61%) as yellow oil. LC-MS: m/z=214.0 [M+H]⁺ (95% purity)

To a mixture of compound 4 (43 mg, 0.2 mmol) and intermediate 9 (52 mg, 0.2 mmol) in DCM (20 mL) was added TEA (41 mg, 0.4 mmol) and 2,4,6-tripropyl-1,3,5,2,4,6-trioxatriphosphorinane-2,4,6-trioxide (254 mg, 0.4 mmol) at 0° C. under N₂, then stirred at room temperature for 2 hours. After consumption of the starting material (by TLC), diluted with water and extracted with DCM. Combined organic extracts were dried over anhydrous MgSO₄ and concentrated under reduced pressure to obtain crude product, which was purified by PREP-HPLC (MeCN/H₂O 3:2) to afford 101-1 (14 mg, 15%) as white solid. LC-MS: m/z=456.2 [M+H]⁺ at RT 5.04 (98.46% purity). ¹H NMR (300 MHz, CD₃OD) δ 7.48 (d, J=8.3 Hz, 1H), 7.37 (t, J=6.4 Hz, 3H), 7.33-7.17 (m, 4H), 4.77-4.46 (m, 4H), 4.29 (dd, J=38.7, 16.5 Hz, 1H), 4.00 (dd, J=10.1, 6.6 Hz, 1H), 3.46 (d, J=17.3 Hz, 1H), 3.13 (dd, J=7.8, 3.9 Hz, 2H), 2.81-2.65 (m, 1H), 2.48 (d, J=6.1 Hz, 1H), 2.33 (dd, J=13.4, 8.2 Hz, 1H), 2.12-1.94 (m, 1H), 1.89-1.53 (m, 5H), 1.04 (dd, J=35.8, 6.7 Hz, 3H).

### Example 102

### (R)-(9H-fluoren-9-yl)methyl 1-cyclobutylethyl((1-(2,4-dimethoxybenzyl)-1H-1,2,3-triazol-4-yl)methyl)carbamate (1)

To a stirring solution of (R)-1-cyclobutyl-N-((1-(2,4-dimethoxybenzyl)-1H-1,2,3-triazol-4-yl)methyl)ethanamine SM1 (400 mg, 1.21 mmol) and DIPEA (230 mg, 1.8 mmol) in dry DCM (10 mL) was added Fmoc-Cl (370 mg, 1.45 mmol) at 0° C. The reaction mixture was then stirred at room temperature for 2 hours and concentrated under reduced pressure to obtain crude product, which was purified by silica gel column chromatography eluting with 50% EtOAc/Hexane to afford compound 1 (550 mg, 83%).

TLC: 50% EtOAc/Hexane (R_{f}: 0.3). LC-MS: m/z 553.2 [M+H]⁺

### (R)-(9H-fluoren-9-yl)methyl (1H-1,2,3-triazol-4-yl)methyl(1-cyclobutylethyl)carbamate (2)

A solution of compound 1 (550 mg, 0.1 mmol) in TFA (6 mL) was heated 70° C. for 1 hour then cooled and concentrated and purified by silica gel column chromatography eluting with 50% EtOAc/Hexane to afford compound 2 (350 mg, 87%).

TLC: 50% EtOAc/Hexane (R_{f}: 0.3). LC-MS: m/z 403.2 [M+H]⁺

### (R)-(9H-fluoren-9-yl)methyl 1-cyclobutylethyl((1-methyl-1H-1,2,3-triazol-4-yl)methyl)carbamate (3)

To a stirring solution of compound 2 (200 mg, 0.5 mmol) and K₂CO₃ (140 mg, 1 mmol) in dry MeCN (2 ml) was added MeI (700 mg, 0.5 mmol) at room temperature. The reaction mixture was stirred at room temperature overnight then concentrated and purified by silica gel column chromatography eluting with 50% EtOAc/Hexane to afford compound 3 (100 mg, 48%). TLC: 50% EtOAc/Hexane (R_{f}: 0.3). LC-MS: m/z=417.2 [M+H]⁺

### (R)-1-cyclobutyl-N-((1-methyl-1H-1,2,3-triazol-4-yl)methyl)ethanamine (4)

To a stirring solution of compound 3 (100 mg, 0.24 mmol) in DMF (1.6 mL) was added piperidine (0.4 ml) at room temperature. The reaction mixture was stirred at room temperature for 1 hour then quenched with H₂O and extracted with EtOAc. Combined organic extracts were dried over anhydrous Na₂SO₄ and concentrated and purified by silica gel column chromatography eluting with 5% MeOH/DCM to afford compound 4 (25 mg, 54%). TLC: 10% MeOH/DCM (R_{f}: 0.4). LC-MS: m/z=194.1 [M+H]⁺

To a stirring solution of compound 4 (25 mg, 0.13 mmol) in DMF (1 mL) was added compound intermediate-9 (33 mg, 0.13 mmol) and DIPEA (34 mg, 0.26 mmol) at room temperature. To this added HATU (60 mg, 0.16 mmol) at room temperature and the reaction mixture was stirred at room temperature for 1 hour. the reaction mixture was diluted with water and extracted with EtOAc. Combined organic extracts were dried over anhydrous Na₂SO₄ and concentrated under reduced pressure to obtain crude product, which was purified by silica gel column chromatography eluting with 3% MeOH/DCM to afford compound 102-1 (10 mg, 20%) as white solid. TLC: 10% MeOH/DCM (Rf: 0.6). LC-MS: m/z=437.2 [M+H]⁺ (98% purity). ¹H NMR (400 MHz, CD₃OD-d4) 67.50 (s, 1H), 7.40-7.28 (m, 3H), 7.24 (s, 1H), 4.73-4.59 (m, 2H), 4.57-4.46 (m, 2H), 4.05 (s, 3H), 3.16-3.12 (m, 2H), 2.76-2.66 (m, 2H), 2.38-2.28 (m, 2H), 2.16-2.05 (m, 2H), 1.78-1.90 (s, 4H), 1.18 (d, J=6.6 Hz, 3H).

### Example 103

### (R)-(9H-fluoren-9-yl)methyl (1-(cyanomethyl)-1H-1,2,3-triazol-4-yl)methyl(1-cyclobutylethyl)carbamate (1)

To a stirring solution of compound SM1 (300 mg, 0.75 mmol) and K₂CO₃ (200 mg, 1.5 mmol) in dry MeCN (2 ml) was added 2-bromoacetonitrile (100 mg, 0.82 mmol) at room temperature. The reaction mixture was stirred at room temperature overnight then concentrated and purified by silica gel column chromatography eluting with 33% EtOAc/Hexane to afford compound 1 (120 mg, 73%), and 150 mg of SM1 was recovered. TLC: 50% EtOAc/Hexane (R_{f}: 0.4). LC-MS: m/z=442.2 [M+H]⁺

### (R)-2-(4-((1-cyclobutylethylamino)methyl)-1H-1,2,3-triazol-1-yl)acetonitrile (2)

To a stirring solution of compound 1 (120 mg, 0.27 mmol) in DMF (1.6 ml) was added piperidine (0.4 ml) at room temperature. The reaction mixture was stirred at room temperature for ¹H then quenched with H₂O and extracted with EtOAc. Combined organic extracts were dried over anhydrous Na₂SO₄ and concentrated and purified by silica gel column chromatography eluting with 5% MeOH/DCM to afford compound 2 (40 mg, 67%). TLC: 10% MeOH/DCM (R_{f}: 0.3). LC-MS: m/z=220.1 [M+H]⁺

To a stirring solution of compound 2 (40 mg, 0.18 mmol) in DMF (1 mL) was added compound intermediate-9 (47 mg, 0.18 mmol) and DIPEA (46 mg, 0.36 mmol) at room temperature. To this added HATU (82 mg, 0.22 mmol) at room temperature and the reaction mixture was stirred at room temperature for 1 hour. the reaction mixture was diluted with water and extracted with EtOAc. Combined organic extracts were dried over anhydrous Na₂SO₄ and concentrated under reduced pressure to obtain crude product, which was purified by silica gel column chromatography eluting with 3% MeOH/DCM to afford compound 103-1 (25 mg, 30%) as white solid. TLC: 10% MeOH/DCM (Rf: 0.6). LC-MS: m/z=462 [M+H]⁺ (98% purity). ¹H NMR (400 MHz, DMSO-d6) δ8.82 (d, J=10.7 Hz, 1H), 7.52 (s, 1H), 7.38-7.21 (m, 4H), 5.87 (d, J=13.7 Hz, 2H), 4.62-4.50 (m, 1H), 4.45-4.31 (m, 3H), 4.04-3.90 (m, 1H), 3.04 (t, J=7.2 Hz, 2H), 2.60-2.50 (m, 1H), 2.25-2.15 (m, 1H), 2.01-1.91 (m, 1H), 1.77-1.43 (m, 6H), 1.06 (d, J=6.4 Hz, 2H), 0.90 (d, J=6.8 Hz, 1H).

### Example 104

### (S)-tert-butyl 4-((1-cyclobutylethylamino)methyl)phenylcarbamate (2)

To a stirring solution of tert-butyl 4-(bromomethyl)phenylcarbamate SM1 (50 mg, 0.175 mmol) in DCM (10 ml), was added (S)-1-cyclobutylethanamine compound 1 (20.8 mg, 0.21 mmol), followed by DIEA (45 mg, 0.35 mmol) at room temperature. The reaction mixture stirred at room temperature for 16 h. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 20% EA/PE to afford compound 2 (30 mg, 56%). TLC: 20% EA/PE (R_{f}: 0.3). LC-MS: m/z=305 [M+H]⁺

### tert-butyl 4-((N—((S)-1-cyclobutylethyl)-2-((S)-2,5-dioxo-2′,3′-dihydrospiro[imidazolidine-4,1′-indene]-1-yl)acetamido)methyl)phenylcarbamate (4)

To a stirring solution of (S)-tert-butyl 4-((1-cyclobutylethylamino)methyl) phenylcarbamate compound 1 (30 mg, 0.1 mmol), in DMF (5 mL) was added (S)-2-(2,5-dioxo-2′,3′-dihydrospiro[imidazolidine-4,1′-indene]-1-yl)acetic acid Intermediate 9 (26 mg, 0.1 mmol), followed by Et₃N (20.2 mg, 0.2 mmol) and HATU (38 mg, 0.12 mmol), After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 20% EA/PE to afford compound 4 (50 mg, 91%). TLC: 20% EA/PE (R_{f}: 0.2). LC-MS: m/z=547 [M+H]⁺

### N-(4-aminobenzyl)-N—((S)-1-cyclobutylethyl)-2-((S)-2,5-dioxo-2′,3′-dihydrospiro[imidazolidine-4,1′-indene]-1-yl)acetamide (5)

To a stirring solution of tert-butyl 4-((N—((S)-1-cyclobutylethyl)-2-((S)-2,5-dioxo-2′,3′-dihydrospiro[imidazolidine-4,1′-indene]-1-yl)acetamido)methyl)phenylcarbamate compound 4 (50 mg, 0.09 mmol) in dry DCM (5 mL) was added TFA (2 mL) and stirred at room temperature for 2 hours. The reaction mixture concentrated under reduced pressure to obtain crude product compound 5 (20 mg, 50%). TLC: 50% EtOAc/Hexane (R_{f}: 0.3). LC-MS: m/z=447 [M+H]⁺

To a stirring solution of compound 5 (20 mg, 0.045 mmol) in 10% HOAc (20 mL) was added KCNO (3.6 mg, 0.045 mmol), and stirred at room temperature for 2 hours. The reaction mixture was diluted with water and extracted with EA. Combined organic extracts were dried over anhydrous Na₂SO₄ and concentrated under reduced pressure to obtain crude product, which was purified by silica gel column chromatography with 50% EA/PE to afford 104-1 (9 mg, 40%) as an off-white solid. LC-MS: m/z=490 [M+H]⁺ at room temperature 1.425 (100% purity). ¹H NMR (301 MHz, DMSO) δ 8.87-8.71 (m, 1H), 8.63-8.40 (m, 1H), 7.42-6.96 (m, 9H), 5.92-5.72 (m, 2H), 4.63-3.90 (m, 5H), 3.03 (s, 3H), 2.33-2.13 (m, 1H), 2.02-1.78 (m, 1H), 1.82-1.56 (m, 3H), 1.33-1.14 (m, 4H), 0.89 (s, 4H).

### Example 105

### N-benzyl-N—((S)-1-cyclopropylethyl)-2-(5-(3-hydroxyprop-1-ynyl)-2′,4′-dioxo-2,3-dihydrospiro[indene-1,5′-ozazolidine]-3′-yl)acetamide

To a mixture of prop-2-yn-1-ol SM (56 mg, 1.0 mmol) and N-benzyl-2-(5-bromo-2′,4′-dioxo-2,3-dihydrospiro[indene-1,5′-ozazolidine]-3′-yl)-N—((S)-1-cyclopropylethyl)acetamide Inter-28 (50 mg, 0.1 mmol) in DMF (2 mL) was added triethylamine (0.5 mL), CuI (10 mg, 0.05 mmol) and then Pd(PPh₃)₄ (12 mg, 0.01 mmol) under argon atmosphere. The mixture was degassed and sealed, which was stirred at 120° C. for 48 h with oil bath. The reaction mixture was diluted with EtOAc (30 mL) and washed with water (10 mL). The organic extracts was concentrated under reduced pressure to obtain crude product, which was purified by Prep-HPLC to afford N-benzyl-N—((S)-1-cyclopropylethyl)-2-(5-(3-hydroxyprop-1-ynyl)-2′,4′-dioxo-2,3-dihydrospiro[indene-1,5′-ozazolidine]-3′-yl)acetamide (5 mg, 11%) as an off-white solid. ¹H NMR (400 MHz, CD₃OD-d4): δ 7.60-7.16 (m, 8H), 4.89-4.36 (m, 6H), 3.87-3.83 (m, 1H), 3.25-3.08 (m, 2H), 2.92-2.74 (m, 1H), 2.65-2.52 (m, 1H), 1.32-1.20 (m, 3H), 1.06-0.82 (m, 1H), 0.70-0.51 (m, 1H), 0.43-0.22 (m, 3H). LC-MS: m/z=472.8 [M+H]⁺ at RT 4.71 (99.00% purity)

To a mixture of intermediate-28 (50 mg, 0.1 mmol) in DMF (2 mL) was added triethylamine (0.5 mL), CuI (10 mg, 0.05 mmol) and then Pd(PPh₃)₄ (12 mg, 0.01 mmol) under argon atmosphere. The mixture was degassed and sealed, which was stirred at 120° C. for 48 h with oil bath. The reaction mixture was diluted with EtOAc (30 mL) and washed with water (10 mL). The organic extracts was concentrated under reduced pressure to obtain crude product, which was purified by Prep-HPLC to afford 105-1 (5 mg, 11%) as an off-white solid. LC-MS: m/z=472.8 [M+H]⁺ at RT 4.71 (99.00% purity). ¹H NMR (400 MHz, CD₃OD-d4): δ 7.60-7.16 (m, 8H), 4.89-4.36 (m, 6H), 3.87-3.83 (m, 1H), 3.25-3.08 (m, 2H), 2.92-2.74 (m, 1H), 2.65-2.52 (m, 1H), 1.32-1.20 (m, 3H), 1.06-0.82 (m, 1H), 0.70-0.51 (m, 1H), 0.43-0.22 (m, 3H).

### Example 106

### N-benzyl-N—((S)-1-cyclopropylethyl)-2-(5′-(3-hydroxy-3-methylbut-1-ynyl)-2,5-dioxo-2′,3′-dihydrospiro[imidazolidine-4,1′-indene]-1-yl)acetamide

To a mixture of prop-2-yn-1-ol SM (84 mg, 1.0 mmol) and N-benzyl-2-(5′-bromo-2,5-dioxo-2′,3′-dihydrospiro[imidazolidine-4,1′-indene]-1-yl)-N—((S)-1-cyclopropylethyl)acetamide Inter-27 (50 mg, 0.1 mmol) in DMF (2 mL) was added triethylamine (0.5 mL), CuI (10 mg, 0.05 mmol) and then Pd(PPh₃)₄ (12 mg, 0.01 mmol) under argon atmosphere. The mixture was degassed and sealed, which was stirred at 150° C. for 48 h with oil bath. The reaction mixture was diluted with EtOAc (20 mL) and washed with water (10 mL). The organic extracts was concentrated under reduced pressure to obtain crude product, which was purified by Prep-HPLC to afford N-benzyl-N—((S)-1-cyclopropylethyl)-2-(5′-(3-hydroxy-3-methylbut-1-ynyl)-2,5-dioxo-2′,3′-dihydrospiro[imidazolidine-4,1′-indene]-1-yl)acetamide (3 mg, 6%) as an off-white solid. ¹H NMR (400 MHz, CD₃OD-d4): δ 7.53-7.16 (m, 8H), 4.80-4.74 (m, 2H), 4.59-4.27 (m, 2H), 3.86-3.80 (m, 1H), 3.20-3.08 (m, 1H), 2.74-2.68 (m, 1H), 2.37-2.32 (m, 1H), 1.57 (s, 6H), 1.30-1.20 (m, 1H), 0.97-0.93 (m, 1H), 0.59-0.55 (m, 1H), 0.33-0.25 (m, 3H). LC-MS: m/z=500.0 [M+H]⁺ at RT 4.51 (97.86% purity)

To a mixture of intermediate-27 (50 mg, 0.1 mmol) in DMF (2 mL) was added triethylamine (0.5 mL), CuI (10 mg, 0.05 mmol) and then Pd(PPh₃)₄ (12 mg, 0.01 mmol) under argon atmosphere. The mixture was degassed and sealed, which was stirred at 150° C. for 48 h with oil bath. The reaction mixture was diluted with EtOAc (20 mL) and washed with water (10 mL). The organic extracts was concentrated under reduced pressure to obtain crude product, which was purified by Prep-HPLC to afford 106-1 (3 mg, 6%) as an off-white solid. LC-MS: m/z=500.0 [M+H]⁺ at RT 4.51 (97.86% purity). ¹H NMR (400 MHz, CD₃OD-d4): δ 7.53-7.16 (m, 8H), 4.80-4.74 (m, 2H), 4.59-4.27 (m, 2H), 3.86-3.80 (m, 1H), 3.20-3.08 (m, 1H), 2.74-2.68 (m, 1H), 2.37-2.32 (m, 1H), 1.57 (s, 6H), 1.30-1.20 (m, 1H), 0.97-0.93 (m, 1H), 0.59-0.55 (m, 1H), 0.33-0.25 (m, 3H).

### Example 107

### (R)-(9H-fluoren-9-yl)methyl (1-(2-amino-2-oxoethyl)-1H-1,2,3-triazol-4-yl)methyl(1-cyclobutylethyl)carbamate (1)

To a stirring solution of compound SM1 (440 mg, 1.1 mmol) and DIPEA (300 mg, 2.2 mmol) in dry DCM (5 ml) and dry MeOH (5 ml) was added 2-bromo-N,N-dimethylacetamide (1700 mg, 1.2 mmol) at room temperature. The reaction mixture was stirred at 60° C. overnight then concentrated and purified by silica gel column chromatography eluting with 6% MeOH/DCM to afford compound 1 (120 mg, 35%), and 100 mg of SM1 was recovered. TLC: 10% MeOH/DCM (R_{f}: 0.25). LC-MS: m/z=460.1 [M+H]⁺

### (R)-2-(4-((1-cyclobutylethylamino)methyl)-1H-1,2,3-triazol-1-yl)-N,N-dimethylacetamide (2)

To a stirring solution of compound 1 (120 mg, 0.3 mmol) in DMF (1.6 mL) was added piperidine (0.4 mL) at room temperature. The reaction mixture was stirred at room temperature for 1 hour then quenched with H₂O and extracted with EtOAc. Combined organic extracts were dried over anhydrous Na₂SO₄ and concentrated and purified by silica gel column chromatography eluting with 10% MeOH/DCM(with 1% NH₄OH) to afford compound 2 (40 mg, 65%). TLC: 10% MeOH/DCM (with 1% NH₄OH) (R_{f}: 0.2). LC-MS: m/z=238.1 [M+H]⁺

To a stirring solution of compound 2 (40 mg, 0.16 mmol) in DMF (1 mL) was added compound intermediate-9 (40 mg, 0.16 mmol) and DIPEA (44 mg, 0.32 mmol) at room temperature. To this added HATU (90 mg, 0.24 mmol) at room temperature and the reaction mixture was stirred at room temperature for 1 hour. the reaction mixture was diluted with water and extracted with EtOAc. Combined organic extracts were dried over anhydrous Na₂SO₄ and concentrated under reduced pressure to obtain crude product, which was purified by silica gel column chromatography eluting with 10% MeOH/DCM(with 1% NH4OH) to afford compound 107-1 (20 mg, 26%) as white solid. TLC: 10% MeOH/DCM(with 1% NH₄OH) (Rf: 0.2). LC-MS: m/z=480.2 [M+H]⁺ (98% purity). ¹H NMR (400 MHz, DMSO-d6) δ 8.81 (d, J=12.8 Hz, 1H), 7.57 (s, 1H), 7.40-7.25 (m, 6H), 5.02 (d, J=13.8 Hz, 2H), 4.61-4.33 (m, 5H), 3.99-3.92 (m, 1H), 3.05 (t, J=6.8 Hz, 2H), 2.29-2.20 (m, 1H), 1.99-1.91 (m, 1H), 1.75-1.35 (m, 6H), 1.10-0.80 (m, 3H).

### Example 108

### 5-(4,4,5,5-tetramethyl-1,3,2-dioxaborolan-2-yl)pyridin-2-ol (3)

A solution of 5-bromopyridin-2-ol SM1 (0.1 g, 0.57 mmol), 4,4,4′,4′,5,5,5′,5′-octamethyl-2,2′-bi(1,3,2-dioxaborolane) SM2 (0.88 g, 3.45 mmol), Tricyclohexyl phosphine (0.032 g, 0.114 mmol), Pd₂(dba)₃ (0.052 g, 0.057 mmol) and KOAc (0.17 g, 1.71 mmol) dissolved with dioxane (10 mL) in sealed tube was stirred at 110° C. overnight. After consumption of the starting material (by LC-MS), the solvent from reaction mixture was removed under reduced pressure, the residue was diluted with brine and extracted with EtOAc. Combined organic extracts were dried over anhydrous Na₂SO₄ and concentrated under reduced pressure to obtain crude product, which was purified by silica gel column chromatography to afford compound 3 (0.08 g, 64%) as a black liquid.

TLC: 50% EA/PE.

### (S)—N-benzyl-N-(1-cyclopropylethyl)-2-(2,5-dioxo-5′-(6-oxo-1,6-dihydropyridin-3-yl)-2′,3′-dihydrospiro[imidazolidine-4,1′-indene]-1-yl)acetamide

A solution of 3 (0.045 g, 0.2 mmol), SM4 (0.1 g, 0.2 mmol), Pd(dppf)₂Cl₂ (0.015 g, 0.01 mmol) and K₂CO₃ (0.083 g, 0.6 mmol) dissolved with dioxane and H₂O (10 mL/1 mL) in sealed tube was stirred at 95° C. overnight. After consumption of the starting material (by LC-MS), the solvent from reaction mixture was removed under reduced pressure, the residue was diluted with brine and extracted with EtOAc. Combined organic extracts were dried over anhydrous Na₂SO₄ and concentrated under reduced pressure to obtain crude product, which was purified by Prep-HPLC [H₂O-CAN(0.05% TFA)] to afford compound 108-1 (0.036 g, 35%) as a white liquid. ¹H NMR (400 MHz, DMSO-d₆): δ 11.89 (s, 1H), 8.80 (d, J=16.9 Hz, 1H), 7.83 (d, J=9.5 Hz, 1H), 7.71 (s, 1H), 7.54-7.15 (m, 8H), 6.44 (d, J=9.3 Hz, 1H), 4.70-4.40 (m, 2H), 4.35-4.08 (m, 2H), 3.72 (s, 1H), 3.07 (d, J=6.5 Hz, 2H), 2.66-2.53 (m, 1H), 2.22 (d, J=13.2 Hz, 1H), 1.23-1.06 (m, 3H), 0.95 (s, 1H), 0.48-0.16 (m, 4H). LC-MS: m/z=511.1 [M+H]⁺ (96.57% purity, 214 nm)

### Example 109

### (S)-methyl 5-bromo-1-isocyanato-2,3-dihydro-1H-indene-1-carboxylate (1)

To a stirring solution of intermediate 3 (1.4 g, 5.2 mmol) in DCM cooled to 0° C. and was added Et₃N (0.8 g, 7.8 mmol) followed by triphosgene (0.61 g, 2.1 mmol). The reaction mixture was stirred under N₂ for 1 hour. After consumption of the starting material (by TLC), the reaction mixture was used to the next step without any purification.

LC-MS: m/z=297.12 [M+H]⁺

### (S)-methyl 1-(3-(2-(benzyl((S)-1-cyclopropylethyl)amino)-2-oxoethyl)ureido)-5-bromo-2,3-dihydro-1H-indene-1-carboxylate (2)

The mixture from previous step was added another equal Et₃N and followed by intermediate 4 (1.2 g, 5.2 mmol) and stirred under N₂ at room temperature overnight, After consumption of the starting material (by TLC), the solvent from reaction mixture was removed under reduced pressure. Obtained residue was used to the next step without any other purification. LC-MS: m/z=528.1/530.1 [M+H]⁺

### N-benzyl-2-((S)-5′-bromo-2,5-dioxo-2′,3′-dihydrospiro[imidazolidine-4,1′-indene]-1-yl)-N—((S)-1-cyclopropylethyl)acetamide (3)

To a stirring solution of compound 2 (1.2 g, 2.7 mmol) in 30 mL THF was added 1N LiOH (8.1 mmol) and the mixture was stirred at room temperature for 3 hours, After consumption of the starting material (by TLC), the mixture was diluted with water and extracted with DCM, Combined organic extracts were dried over anhydrous Na₂SO₄ and concentrated under reduced pressure to obtain crude product, which was purified by silica gel column chromatography to afford compound 3 (1.1 g, 55%) as an off-white solid. LC-MS: m/z=496.12 [M+H]⁺

To a stirring solution of compound 3 (20 mg, 0.04 mmol) in dioxane/H₂O (10 mL) was added NaHCO3 (10 mg, 0.08 mmol), dppf PdCl₂ (5.3 mg, 0.004 mmol) and ¹H-pyrazol-4-ylboronic acid (6.8 mg, 0.06 mmol) and refluxing under N₂ for 2 hours. After consumption of the starting material (by TLC), the mixture was diluted with water and extracted with DCM, Combined organic extracts were dried over anhydrous Na2SO4 and concentrated under reduced pressure to obtain crude product, which was purified by silica gel column chromatography to afford 109-1 (12 mg, 65%) as an off-white solid. LC-MS: m/z=484.1 [M+H]⁺ at RT 1.51 (100% purity). ¹H NMR (301 MHz, CD₃OD) δ 7.68 (dd, J=15.9, 8.1 Hz, 3H), 7.41 (dt, J=15.7, 7.5 Hz, 4H), 7.34-7.11 (m, 2H), 6.70 (s, 1H), 4.83-4.68 (m, 2H), 4.49 (d, J=7.5 Hz, 1H), 4.35 (s, 1H), 3.89-3.81 (m, 0.5H), 3.38 (s, 0.5H), 3.25-3.10 (m, 2H), 2.75 (ddd, J=13.2, 7.9, 5.0 Hz, 1H), 2.37 (dd, J=14.3, 7.0 Hz, 1H), 1.26 (dd, J=24.0, 6.7 Hz, 3H), 0.96 (s, 1H), 0.57 (d, J=7.4 Hz, 1H), 0.46-0.17 (m, 3H).

### Example 110

### N-benzyl-N—((S)-1-cyclopropylethyl)-2-(5-(N-(methylsulfonyl)methylsulfonamido)-2′,4′-dioxo-2,3-dihydrospiro[indene-1,5′-ozazolidine]-3′-yl)acetamide (1)

To a stirring solution of intermediate 18 (0.05 g, 0.115 mmol), TEA(35 mg, 0.345 mmol) in DCM (6 mL) was added MsCl (26 mg, 0.23 mmol) at 0° C., the mixture was stirred for 1 hour. After consumption of SM1 (by TLC), the solvent from reaction mixture was removed under reduced pressure, the residue was diluted with brine and extracted with EtOAc. Combined organic extracts were dried over anhydrous Na₂SO₄ and concentrated under reduced pressure to obtain crude product(50 mg), which was used directly for the subsequent step. TLC: 50% EA/PE

### N-benzyl-N—((S)-1-cyclopropylethyl)-2-(5-(methylsulfonamido)-2′,4′-dioxo-2,3-dihydrospiro[indene-1,5′-ozazolidine]-3′-yl)acetamide

A solution of compound 1 (50 mg), K₂CO₃ (35 mg, 0.255 mmol) in THF (5 mL) and H₂O (5 mL) was stirred for 2 hours. After consumption of the starting material (by LC-MS), the solvent from reaction mixture was removed under reduced pressure, the residue was diluted with brine and extracted with EtOAc. Combined organic extracts were dried over anhydrous Na₂SO₄ and concentrated under reduced pressure to obtain crude product, which was purified by Prep-HPLC [H₂O-CAN(0.05% TFA)] to afford compound 110-1 (0.01 g, 18%) as a white liquid. ¹H NMR (400 MHz, MeOD-d₄): δ 7.57-7.35 (m, 4H), 7.26-7.16 (m, 4H), 4.82-4.43 (m, 4H), 3.85 (ddd, J=10.3, 6.8, 3.9 Hz, 1H), 3.26-3.08 (m, 2H), 2.99 (d, J=2.1 Hz, 3H), 2.85-2.74 (m, 1H), 2.62-2.53 (m, 1H), 1.29 (d, J=23.7 Hz, 3H), 0.98 (s, 1H), 0.65-0.55 (m, 1H), 0.41-0.26 (m, 3H). LC-MS: m/z=511.8 [M+H]⁺ (93.70% purity, 214 nm)

### Example 111

### 2-(5′-acetamido-2,5-dioxo-2′,3′-dihydrospiro[imidazolidine-4,1′-indene]-1-yl)-(S)—N-benzyl-N-(1-cyclopropylethyl)acetamide (3)

A solution of 2-(5′-acetamido-2,5-dioxo-2′,3′-dihydrospiro[imidazolidine-4,1′-indene]-1-yl)acetic acid SM1 (180 mg, 0.57 mmol), (S)—N-benzyl-1-cyclopropylethanamine SM2 (99 mg, 0.57 mmol), HATU (433 mg, 1.14 mmol), DIPEA (147 mg, 1.14 mmol) in DCM (10 mL) was stirred for 2 hours. After consumption of the starting material (by LC-MS), the solvent from reaction mixture was removed under reduced pressure, the residue was diluted with brine and extracted with EtOAc. Combined organic extracts were dried over anhydrous Na₂SO₄ and concentrated under reduced pressure to obtain crude product, which was purified by silica gel column chromatography to afford compound 3 (160 mg, 60%) as a yellow solid. TLC: 50% PE/EA

### 2-(5′-amino-2,5-dioxo-2′,3′-dihydrospiro[imidazolidine-4,1′-indene]-1-yl)-(S)—N-benzyl-N-(1-cyclopropylethyl)acetamide (4)

SM3 (100 mg, 0.21 mmol) was dissolved with MeOH/THF/H₂O (5 mL/5 mL/5 mL), and 37% HCl (1 mL) was added. The mixture was heated to 60° C. and stirred for 2 hours. After consumption of the SM3 (by LC-MS), the solvent from reaction mixture was removed under reduced pressure. the residue was diluted with sat NaCl aqueous and extracted with EtOAc. Combined organic extracts were dried over anhydrous Na₂SO₄ and concentrated under reduced pressure to obtain crude product, which was purified by silica gel column chromatography to afford compound 4 (74 mg, 81%) as a yellow liquid. TLC: 50% PE/EA

### (S)—N-benzyl-N-(1-cyclopropylethyl)-2-(5′-(N-(methylsulfonyl)methylsulfonamido)-2,5-dioxo-2′,3′-dihydrospiro[imidazolidine-4,1′-indene]-1-yl)acetamide (5)

To a stirring solution of SM4 (0.05 g, 0.115 mmol), TEA(35 mg, 0.345 mmol) in DCM (6 mL) was added MsCl (26 mg, 0.23 mmol) at 0° C., the mixture was stirred for 1 hour. After consumption of SM4 (by TLC), the solvent from reaction mixture was removed under reduced pressure, the residue was diluted with brine and extracted with EtOAc. Combined organic extracts were dried over anhydrous Na₂SO₄ and concentrated under reduced pressure to obtain crude product, which was used directly for the subsequent step. TLC: 50% EA/PE

### (S)—N-benzyl-N-(1-cyclopropylethyl)-2-(5′-(methylsulfonamido)-2,5-dioxo-2′,3′-dihydrospiro[imidazolidine-4,1′-indene]-1-yl)acetamide

A solution of SM5 (the crude), K₂CO₃ (35 mg, 0.255 mmol) in THF (5 mL) and H₂O (5 mL) was stirred for 2 hours. After consumption of the starting material (by LC-MS), the solvent from reaction mixture was removed under reduced pressure, the residue was diluted with brine and extracted with EtOAc. Combined organic extracts were dried over anhydrous Na₂SO₄ and concentrated under reduced pressure to obtain crude product, which was purified by Prep-HPLC [H₂O-ACN(0.05% TFA)] to afford compound 111-1 (0.016 g, 37%) as a white liquid. ¹H NMR (400 MHz, MeOD-d₄): δ 7.49-7.10 (m, 8H), 4.81-4.34 (m, 4H), 3.82 (dd, J=6.6, 3.4 Hz, 1H), 3.14 (t, J=6.8 Hz, 2H), 2.97 (s, 3H), 2.79-2.70 (m, 1H), 2.40-2.30 (m, 1H), 1.33-1.21 (m, 3H), 0.97 (d, J=4.4 Hz, 1H), 0.64-0.54 (m, 1H), 0.42-0.24 (m, 3H). LC-MS: m/z=510.9 [M+H]⁺ (96.58% purity, 214 nm)

### Example 112

### N-(2,5-dioxo-2′,3′-dihydrospiro[imidazolidine-4,1′-indene]-5′-yl)acetamide (1)

To a stirring solution of SM1 (1.9 g, 10 mmol) in EtOH (15 mL), H₂O (15 mL) was added (NH₄)₂CO₃ (4.8 g, 50 mmol) followed by potassium cyanide (1.3 g, 20 mmol) at room temperature. The reaction mixture was heated to 80° C. for 72 hours. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 1 (0.9 g, 35%) as a yellow solid. LC-MS: m/z 260 [M+H]⁺

### tert-Butyl 2-(5′-acetamido-2,5-dioxo-2′,3′-dihydrospiro[imidazolidine-4,1′-indene]-1-yl) acetate (2)

To a stirring solution of compound 1 (655 mg, 2.53 mmol) in DMF (5 mL) was added K₂CO₃ (524.4 mg, 3.8 mmol) followed by tert-butyl 2-bromoacetate (518.7 mg, 2.66 mmol) at room temperature and stirred at room temperature for 1 hour. The reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 2 (616 mg, 65%) as a yellow solid. LC-MS: m/z=375 [M+H]⁺

### 2-(5′-Acetamido-2,5-dioxo-2′,3′-dihydrospiro[imidazolidine-4,1′-indene]-1-yl)acetic acid (3)

To a stirring solution of compound 2 (616 mg, 1.65 mmol) in DCM (3 mL) was added TFA (3 mL) at room temperature and stirred at room temperature for 2 hours. After consumption of the starting material (by TLC), the reaction mixture was concentrated under reduced pressure to obtain crude compound 3 (500 mg, 96%) as a yellow solid. LC-MS: m/z=318 [M+H]⁺

### 2-(5′-Acetamido-2,5-dioxo-2′,3′-dihydrospiro[imidazolidine-4,1′-indene]-1-yl)-N-benzyl-N—((S)-1-cyclopropylethyl)acetamide (4)

To a stirring solution of compound 3 (106.8 mg, 0.337 mmol) in DMF (1 mL) was added Intermediate 24 (59 mg, 0.337 mmol) and DIPEA (96 mg, 0.741 mmol) followed by HATU (141 mg, 0.371 mmol) at room temperature and stirred at room temperature for 1 hour. The reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 4 (150 mg, 94%) as a yellow solid. LC-MS: m/z=475 [M+H]⁺

### 2-(5′-Amino-2,5-dioxo-2′,3′-dihydrospiro[imidazolidine-4,1′-indene]-1-yl)-N-benzyl-N—((S)-1-cyclopropylethyl)acetamide (5)

To a stirring solution of compound 4 (150 mg, 0.316 mmol) in MeOH (4 mL) was added con. HCl (1 mL) followed by H₂O (1 mL) at room temperature. The reaction mixture was heated to 80° C. for 1 hour. After consumption of the starting material (by TLC), the resulting mixture was cooled to room temperature, neutralized by 4M NaOH solution and the precipitate was filtered and dried to afford compound 5 (85 mg, 62%) as an yellow solid. LC-MS: m/z=433 [M+H]⁺

To a stirring solution of compound 5 (80 mg, 0.185 mmol) in formic acid (1 mL) was added acetic anhydride (1 mL). The reaction mixture was stirred at 60° C. for 2 hours and concentrated. The residue was neutralized by sat. NaHCO3 solution, extracted with EtOAc. Combined organic extracts were dried over anhydrous Na2SO4 and concentrated under reduced pressure to obtain crude product, which was purified by Prep-HPLC to afford 112-1 (21 mg, 25%) as a white solid. LC-MS: m/z=461.1 [M+H]⁺. ¹H NMR (400 MHz, DMSO-d6): δ 10.32-10.16 (m, 1H), 9.99 (s, 1H), 8.75 (dd, J=17.0, 6.8 Hz, 1H), 8.27 (s, 1H), 7.63 (s, 1H), 7.46-7.04 (m, 7H), 4.89-4.74 (m, 2H), 4.43-4.18 (m, 2H), 3.72 (s, 1H), 3.00 (d, J=6.0 Hz, 2H), 2.26-2.04 (m, 2H), 1.22-1.07 (m, 3H), 0.94 (s, 1H), 0.42 (m, 2H), 0.22 (m, 2H).

### Example 113

### N-benzyl-2-(5′-cyano-2,5-dioxo-2′,3′-dihydrospiro[imidazolidine-4,1′-indene]-1-yl)-N—((S)-1-cyclopropylethyl)acetamide (compound 1)

To a mixture of N-benzyl-2-(5′-bromo-2,5-dioxo-2′,3′-dihydrospiro[imidazolidine-4,1′-indene]-1-yl)-N—((S)-1-cyclopropylethyl)acetamide Intermediate-27 (230 mg, 0.46 mmol) in DMF (3 mL) was added Zn(CN)₂ (162 mg, 1.38 mmol) and then Pd(PPh₃)₄ (58 mg, 0.05 mmol) under argon atmosphere. The mixture was degassed and sealed, which was stirred at 120° C. for 1 hour under microwave. The reaction mixture was diluted with ethyl acetate (30 mL) and washed with water (10 mL). The organic phase was concentrated under reduced pressure to obtain crude product, which was purified by Pre-TLC to afford N-benzyl-2-(5′-cyano-2,5-dioxo-2′,3′-dihydrospiro[imidazolidine-4,1′-indene]-1-yl)-N—((S)-1-cyclopropylethyl)acetamide compound 1 (200 mg, 98%) as an off-white solid. LC-MS: m/z=443.0 [M+H]⁺

### N-benzyl-N—((S)-1-cyclopropylethyl)-2-(2,5-dioxo-5′-(2H-tetrazol-5-yl)-2′,3′-dihydrospiro[imidazolidine-4,1′-indene]-1-yl)acetamide

To a mixture of N-benzyl-2-(5′-cyano-2,5-dioxo-2′,3′-dihydrospiro[imidazolidine-4,1′-indene]-1-yl)-N—((S)-1-cyclopropylethyl)acetamide compound 1 (120 mg, 0.25 mmol) and NH₄Cl (4 mg, 0.075 mmol) in DMF (3 mL) was added NaN₃ (146 mg, 0.75 mmol) under argon atmosphere. The mixture was degassed and stirred at 125° C. for 48 h with oil bath. Water was added and the mixture was acidified to pH=1 with concentrated HCl. The mixture was extracted with EtOAc (15 mL×2), combined the organic phase and washed with water (10 mL). The organic phase was concentrated, the residue obtained was dissolved in 0.5N NaOH (10 mL), the above solution was washed with ethyl acetate (10 mL×2). The aqueous phase was acidified to pH=1 with concentrated HCl and then extracted with ethyl acetate (15 mL×2), combined the organic phase and concentrated to afford N-benzyl-N—((S)-1-cyclopropylethyl)-2-(2,5-dioxo-5′-(2H-tetrazol-5-yl)-2′,3′-dihydrospiro[imidazolidine-4,1′-indene]-1-yl)acetamide (80 mg, 66%) as an off-white solid. ¹H NMR (300 MHz, CD₃OD-d4): δ 8.02 (s, 1H), 7.94 (t, J=8.0 Hz, 1H), 7.63 (d, J=8.0 Hz, 1H), 7.47 (d, J=8.0 Hz, 1H), 7.40 (t, J=8.0 Hz, 1H), 7.30-7.12 (m, 3H), 4.84-4.75 (m, 2H), 4.60-4.25 (m, 2H), 3.92-3.78 (m, 1H), 3.30-3.16 (m, 2H), 2.87-2.73 (m, 1H), 2.50-2.35 (m, 1H), 1.38-1.15 (m, 3H), 1.02-0.85 (m, 1H), 0.69-0.49 (m, 1H), 0.45-0.22 (m, 3H). LC-MS: m/z=486.1 [M+H]⁺ at RT 3.96 (93.40% purity)

To a mixture of N-benzyl-2-(5′-cyano-2,5-dioxo-2′,3′-dihydrospiro[imidazolidine-4,1′-indene]-1-yl)-N—((S)-1-cyclopropylethyl)acetamide compound 1 (120 mg, 0.25 mmol) and NH₄Cl (4 mg, 0.075 mmol) in DMF (3 mL) was added NaN₃ (146 mg, 0.75 mmol) under argon atmosphere. The mixture was degassed and stirred at 125° C. for 48 h with oil bath. Water was added and the mixture was acidified to pH=1 with conc. HCl. The mixture was extracted with EtOAc (15 mL×2), combined the organic phase and washed with water (10 mL). The organic phase was concentrated, the residue obtained was dissolved in 0.5N NaOH (10 mL), the above solution was washed with ethyl acetate (10 mL×2). The aqueous phase was acidified to pH=1 with conc. HCl and then extracted with ethyl acetate (15 mL×2), combined the organic phase and concentrated to afford 113-1 (80 mg, 66%) as an off-white solid. LC-MS: m/z=486.1 [M+H]⁺ at RT 3.96 (93.40% purity). ¹H NMR (400 MHz, CD₃OD-d4): δ 8.02 (s, 1H), 7.94 (t, J=8.0 Hz, 1H), 7.63 (d, J=8.0 Hz, 1H), 7.47 (d, J=8.0 Hz, 1H), 7.40 (t, J=8.0 Hz, 1H), 7.30-7.12 (m, 3H), 4.84-4.75 (m, 2H), 4.60-4.25 (m, 2H), 3.92-3.78 (m, 1H), 3.30-3.16 (m, 2H), 2.87-2.73 (m, 1H), 2.50-2.35 (m, 1H), 1.38-1.15 (m, 3H), 1.02-0.85 (m, 1H), 0.69-0.49 (m, 1H), 0.45-0.22 (m, 3H).

### Example 114

To a stirring solution of compound 95-1 (38 mg, 0.088 mmol) in MeOH (2 mL) was added AcONa (16 mg, 0.019 mmol) followed H₂NOH HCl(8 mg, 0.1 mmol). The mixture was stirred at room temperature overnight. The mixture was filtered and dried to afford 114-1 (30 mg, 77%). LC-MS: m/z=447 [M+H]⁺ at RT 1.50 (100% purity). ¹H NMR (400 MHz, DMSO-d6): δ 11.34 (dd, J=22.1, 6.8 Hz, ¹H), 8.88 (d, J=17.5 Hz, 1H), 7.68-7.61 (m, 1H), 7.56-7.42 (m, 3H), 7.42-7.34 (m, 2H), 7.32-7.12 (m, 2H), 4.75 (s, 1H), 4.64 (d, J=8.8 Hz, 1H), 4.45 (d, J=6.2 Hz, 1H), 4.37-4.12 (m, 2H), 3.74 (d, J=7.1 Hz, 1H), 3.42-3.35 (m, 1H), 3.32-3.16 (m, 1H), 2.93 (dd, J=18.6, 15.7 Hz, 1H), 1.25-1.04 (m, 3H), 0.94 (s, 1H), 0.55-0.08 (m, 4H).

### Example 115

To a stirring solution of compound 95-1 (35 mg, 0.081 mmol) in MeOH (2 mL) was added AcONa (15 mg, 0.019 mmol) followed H₂NOMe HCl(8 mg, 0.1 mmol). The mixture was stirred at room temperature overnight. The solvent was removed under reduced pressure. The residue was purified by Prep-HPLC to afford compound 115-1 (15 mg, 42%)) as an off-white solid. LC-MS: m/z=461 [M+H]⁺ at RT 1.61 (100% purity). ¹H NMR (400 MHz, DMSO-d6): δ 11.38-11.31 (m, 1H), 8.90-8.86 (m, 1H), 7.70-7.63 (m, 1H), 7.63-7.46 (m, 3H), 7.43-7.34 (m, 2H), 7.31-7.15 (m, 2H), 4.75 (s, 1H), 4.64 (d, J=9.4 Hz, 1H), 4.53-4.41 (m, 1H), 4.38-4.13 (m, 2H), 3.95 (dd, J=4.1, 2.1 Hz, 3H), 3.73 (d, J=7.0 Hz, 1H), 3.38 (d, J=9.5 Hz, 1H), 3.27 (ddd, J=34.6, 14.6, 6.4 Hz, 1H), 2.97 (ddd, J=18.8, 15.4, 10.1 Hz, 1H), 1.24-1.04 (m, 3H), 0.94 (dd, J=8.3, 4.8 Hz, 1H), 0.57-0.08 (m, 4H).

### Example 116

### (S)—N-benzyl-2-(5-cyano-2′,4′-dioxo-2,3-dihydrospiro[indene-1,5′-ozazolidine]-3′-yl)-N-(1-cyclopropylethyl)acetamide (compound 1)

To a mixture of (S)—N-benzyl-2-(5-bromo-2′,4′-dioxo-2,3-dihydrospiro[indene-1,5′-ozazolidine]-3′-yl)-N-(1-cyclopropylethyl)acetamide Intermediate-28 (200 mg, 0.45 mmol) in DMF (3 mL) was added Zn(CN)₂ (160 mg, 1.36 mmol) and then Pd(PPh₃)₄ (58 mg, 0.05 mmol) under argon atmosphere. The mixture was degassed and sealed, which was stirred at 160° C. for 30 min under microwave. The reaction mixture was diluted with ethyl acetate (30 mL) and washed with water (10 mL) and then brine (10 mL). The organic phase was concentrated under reduced pressure to obtain crude product, which was purified by column chromatography (petroleum ether-ethyl acetate=3:1) to afford (S)—N-benzyl-2-(5-cyano-2′,4′-dioxo-2,3-dihydrospiro[indene-1,5′-ozazolidine]-3′-yl)-N-(1-cyclopropylethyl)acetamide compound 1 (110 mg, 55%) as an off-white solid. LC-MS: m/z=443.8 [M+H]⁺

### (S)-3′-(2-(benzyl(1-cyclopropylethyl)amino)-2-oxoethyl)-2′,4′-dioxo-2,3-dihydrospiro[indene-1,5′-ozazolidine]-5-carboxamide

To a mixture of (S)—N-benzyl-2-(5-cyano-2′,4′-dioxo-2,3-dihydrospiro[indene-1,5′-ozazolidine]-3′-yl)-N-(1-cyclopropylethyl)acetamide compound 1 (110 mg, 0.25 mmol) and K₂CO₃ (70 mg, 0.5 mmol) in methanol (5 mL) was added 30% H₂O₂ (10 drops) at 0° C. The mixture was warmed to room temperature and stirred for 10 min. The mixture was quenched with Na₂SO₃ (sat, aq, 1 mL) and then evaporated to remove organic solvent. The mixture was diluted with ethyl acetate (40 mL) and washed with water (10 mL). The organic phase was dried over anhydrous Na₂SO₄ and concentrated to give crude product, which was purified by Prep-HPLC to afford (S)-3′-(2-(benzyl(1-cyclopropylethyl)amino)-2-oxoethyl)-2′,4′-dioxo-2,3-dihydrospiro[indene-1,5′-ozazolidine]-5-carboxamide (65 mg, 57%) as an off-white solid. ¹H NMR (400 MHz, CD₃OD-d4): δ 7.90 (s, 1H), 7.82 (t, J=8.0 Hz, 1H), 7.64 (d, J=8.0 Hz, 1H), 7.47 (d, J=8.0 Hz, 1H), 7.44 (t, J=8.0 Hz, 1H), 7.37-7.18 (m, 3H), 4.87-4.39 (m, 4H), 3.87-3.83 (m, 1H), 3.31-3.13 (m, 2H), 2.92-2.78 (m, 1H), 2.70-2.57 (m, 1H), 1.32-1.25 (m, 3H), 1.08-0.93 (m, 1H), 0.70-0.55 (m, 1H), 0.45-0.24 (m, 3H). LC-MS: m/z=462.1 [M+H]⁺ at RT 4.26 (99.62% purity)

To a mixture of compound 1 (110 mg, 0.25 mmol) and K₂CO₃ (70 mg, 0.5 mmol) in methanol (5 mL) was added 30% H₂O₂ (10 drops) at 0° C. The mixture was warmed to room temperature and stirred for 10 min. The mixture was quenched with Na₂SO₃ (sat, aq, 1 mL) and then evaporated to remove organic solvent. The mixture was diluted with ethyl acetate (40 mL) and washed with water (10 mL). The organic phase was dried over anhydrous Na₂SO₄ and concentrated to give crude product, which was purified by Prep-HPLC to afford 116-1 (65 mg, 57%) as an off-white solid. LC-MS: m/z=462.1 [M+H]⁺ at RT 4.26 (99.62% purity). ¹H NMR (400 MHz, CD₃OD-d4): δ 7.90 (s, 1H), 7.82 (t, J=8.0 Hz, 1H), 7.64 (d, J=8.0 Hz, 1H), 7.47 (d, J=8.0 Hz, 1H), 7.44 (t, J=8.0 Hz, 1H), 7.37-7.18 (m, 3H), 4.87-4.39 (m, 4H), 3.87-3.83 (m, 1H), 3.31-3.13 (m, 2H), 2.92-2.78 (m, 1H), 2.70-2.57 (m, 1H), 1.32-1.25 (m, 3H), 1.08-0.93 (m, 1H), 0.70-0.55 (m, 1H), 0.45-0.24 (m, 3H).

### Example 117

### N-benzyl-N—((S)-1-cyclopropylethyl)-2-((S)-2,5-dioxo-5′-(6-oxo-1,6-dihydropyridin-3-yl)-2′,3′-dihydrospiro[imidazolidine-4,1′-indene]-1-yl)acetamide

A solution of 5-(4,4,5,5-tetramethyl-1,3,2-dioxaborolan-2-yl)pyridin-2-ol SM1 (0.05 g, 0.23 mmol), N-benzyl-2-((S)-5′-bromo-2,5-dioxo-2′,3′-dihydrospiro[imidazolidine-4,1′-indene]-1-yl)-N—((S)-1-cyclopropylethyl)acetamide SM2 (0.112 g, 0.23 mmol), Pd(dppf)₂Cl₂ (0.085 g, 0.0115 mmol) and K₂CO₃ (0.095 g, 0.69 mmol) dissolved with dioxane and H₂O (10 mL/1 mL) in sealed tube was stirred at 90° C. overnight. After consumption of the starting material (by LC-MS), the solvent from reaction mixture was removed under reduced pressure, the residue was diluted with brine and extracted with EtOAc. Combined organic extracts were dried over anhydrous Na₂SO₄ and concentrated under reduced pressure to obtain crude product, which was purified by Prep-HPLC [H₂O-ACN(0.05% TFA)] to afford compound 117-1 (0.046 g, 39%) as a white liquid. ¹H NMR (400 MHz, DMSO-d₆): δ 12.32-11.56 (m, 1H), 8.81 (d, J=17.1 Hz, 1H), 7.84 (d, J=9.1 Hz, 1H), 7.72 (s, 1H), 7.56-7.15 (m, 8H), 6.44 (d, J=8.8 Hz, 1H), 4.75 (s, 1H), 4.63 (s, 1H) 4.47-4.17 (m, 2H), 3.72 (s, 1H), 3.07 (d, J=5.9 Hz, 2H), 2.56 (d, J=6.5 Hz, 1H), 2.24 (s, 1H), 1.15 (dd, J=31.3, 6.0 Hz, 3H), 0.95 (s, 1H), 0.54-0.12 (m, 4H). LC-MS: m/z=511.2 [M+H]⁺ (100.00% purity, 214 nm)

### Example 118

### (S)-methyl 5-bromo-1-isocyanato-2,3-dihydro-1H-indene-1-carboxylate (1)

To a stirring solution of intermediate 3 (1.4 g, 5.2 mmol) in DCM cooled to 0° C. and was added Et₃N (0.8 g, 7.8 mmol) followed by triphosgene (0.61 g, 2.1 mmol). The reaction mixture was stirred under N₂ for 1 hour. After consumption of the starting material (by TLC), the reaction mixture was used to the next step without any purification.

LC-MS: m/z=297.12 [M+H]⁺

### (S)-methyl 1-(3-(2-(benzyl((S)-1-cyclopropylethyl)amino)-2-oxoethyl)ureido)-5-bromo-2,3-dihydro-1H-indene-1-carboxylate (2)

The mixture was added another equal Et₃N and followed by intermediate 4 (1.2 g, 5.2 mmol) and stirred under N₂ at room temperature overnight, After consumption of the starting material (by TLC), the solvent from reaction mixture was removed under reduced pressure. Obtained residue was used to the next step without any other purification. LC-MS: m/z=528.1/530.1 [M+H]⁺

### N-benzyl-2-((S)-5′-bromo-2,5-dioxo-2′,3′-dihydrospiro[imidazolidine-4,1′-indene]-1-yl)-N—((S)-1-cyclopropylethyl)acetamide (3)

To a stirring solution of compound 2 (1.2 g, 2.7 mmol) in 30 ml THF was added 1N LiOH (8.1 mmol) and the mixture was stirred at room temperature for 3 hours, After consumption of the starting material (by TLC), the mixture was diluted with water and extracted with DCM, Combined organic extracts were dried over anhydrous Na₂SO₄ and concentrated under reduced pressure to obtain crude product, which was purified by silica gel column chromatography to afford compound 3 (1.1 g, 55%) as an off-white solid. LC-MS: m/z=496.1/498.1 [M+H]⁺

To a stirring solution of compound 3 (60 mg, 0.16 mmol) in DMF/Et₃N (6 ml, v/v=2/1) was added prop-2-yn-1-ol (0.4 mL, 2.1 mmol) followed by Pd(PPh₃)₄ (3.5 mg, 0.07 mmol) and CuI (2.0 mg, 0.014 mmol) at room temperature. The reaction mixture was refluxing under N₂ overnight. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford 118-1 (10 mg, 21%). LC-MS: m/z=472.1 [M+H]⁺ at RT 1.52 (100% purity). ¹H NMR (400 MHz, MeOD) δ 7.56-7.07 (m, 8H), 4.79 (d, J=13.9 Hz, 2H), 4.75 (d, J=12.8 Hz, 0.5H), 4.49 (d, J=12.1 Hz, 0.5H), 4.40 (t, J=17.0 Hz, 3H), 3.89-3.80 (m, 1H), 3.25-3.00 (m, 2H), 2.81-2.69 (m, 1H), 2.35 (dd, J=13.4, 7.4 Hz, 1H), 1.27 (dd, J=33.0, 6.7 Hz, 3H), 0.92 (s, 1H), 0.63-0.53 (m, 1H), 0.44-0.19 (m, 3H).

### Example 119

### (S)-methyl 5-bromo-1-isocyanato-2,3-dihydro-1H-indene-1-carboxylate (1)

To a stirring solution of intermediate 3 (1.4 g, 5.2 mmol) in DCM cooled to 0° C. and was added Et₃N (0.8 g, 7.8 mmol) followed by triphosgene (0.61 g, 2.1 mmol). The reaction mixture was stirred under N₂ for 1 hour. After consumption of the starting material (by TLC), the reaction mixture was used to the next step without any purification.

LC-MS: m/z=297.12 [M+H]⁺

### (S)-methyl 1-(3-(2-(benzyl((S)-1-cyclopropylethyl)amino)-2-oxoethyl)ureido)-5-bromo-2,3-dihydro-1H-indene-1-carboxylate (2)

The mixture was added another equal Et₃N and followed by intermediate 4 (1.2 g, 5.2 mmol) and stirred under N₂ at room temperature overnight, After consumption of the starting material (by TLC), the solvent from reaction mixture was removed under reduced pressure. Obtained residue was used to the next step without any other purification. LC-MS: m/z=528.1/530.1 [M+H]⁺

### N-benzyl-2-((S)-5′-bromo-2,5-dioxo-2′,3′-dihydrospiro[imidazolidine-4,1′-indene]-1-yl)-N—((S)-1-cyclopropylethyl)acetamide (3)

To a stirring solution of compound 2 (1.2 g, 2.7 mmol) in 30 mL THF was added 1N LiOH (8.1 mmol) and the mixture was stirred at room temperature for 3 hours, After consumption of the starting material (by TLC), the mixture was diluted with water and extracted with DCM, Combined organic extracts were dried over anhydrous Na₂SO₄ and concentrated under reduced pressure to obtain crude product, which was purified by silica gel column chromatography to afford compound 3 (1.1 g, 55%) as an off-white solid. LC-MS: m/z=496.1/498.1 [M+H]⁺

### N-benzyl-N—((S)-1-cyclopropylethyl)-2-((S)-2,5-dioxo-5′-((trimethylsilyl)ethynyl)-2′,3′-dihydrospiro[imidazolidine-4,1′-indene]-1-yl)acetamide (4)

To a stirring solution of compound 3 (80 mg, 0.16 mmol) in DMF/Et₃N (6 ml, v/v=2/1) was added ethynyltrimethylsilane (0.4 mL, 2.1 mmol) followed by Pd(PPh₃)₄ (3.5 mg, 0.07 mmol) and CuI (2.0 mg, 0.014 mmol) at room temperature. The reaction mixture was refluxing under N₂ overnight. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 4 (0.2 g, 41%). LC-MS: m/z=528.2 [M+H]⁺

### N-benzyl-N—((S)-1-cyclopropylethyl)-2-((S)-5′-ethynyl-2,5-dioxo-2′,3′-dihydrospiro[imidazolidine-4,1′-indene]-1-yl)acetamide (5)

To a stirring solution of compound 4 (0.2 g, 0.67 mmol) in DMF (10 mL) was added K₂CO₃ (300 mg, 1.34 mmol) at room temperature and stirred for 15 min. The reaction mixture was stirred for 1 hour. The solvent from the reaction was removed under reduced pressure. Obtained residue was purified by silica gel column chromatography eluting with 5% DCM/MeOH to afford compound 5 (40 mg, 34%) as an off-white solid.

To a stirring solution of compd-5 (40 mg, 0.1 mmol) in DMSO (2 mL) was added NaN₃ (26 mg, 0.2 mmol) and CuSO₄ 5H2O (0.14 mL, 1M) followed by ascorbate sodium (0.10 mL, 1M). The reaction mixture was stirred at 70° C. for 1 hour. The solvent from the reaction was concentrated under reduced pressure to obtain crude product, which was purified by silica gel column chromatography to afford 119-1 (15 mg, 41%) as an off-white solid. LC-MS: m/z=485.1 [M+H]⁺ at RT 1.48 (100% purity). ¹H NMR (400 MHz, MeOD) δ 8.20 (s, 1H), 7.78 (dd, J=21.3, 13.6 Hz, 2H), 7.55-7.36 (m, 4H), 7.37-7.16 (m, 2H), 4.86-4.70 (m, 2H), 4.63-4.29 (m, 2H), 3.84 (dd, J=9.6, 6.9 Hz, 0.5H), 3.39 (s, 0.5H), 3.22 (dt, J=8.3, 5.8 Hz, 2H), 2.89-2.71 (m, 1H), 2.48-2.31 (m, 1H), 1.28 (dd, J=31.9, 6.7 Hz, 3H), 0.95 (dd, J=14.4, 6.7 Hz, 1H), 0.66-0.52 (m, 1H), 0.46-0.21 (m, 3H).

### Example 120

### N-benzyl-N—((S)-1-cyclopropylethyl)-2-(5-(morpholinomethyl)-2′,4′-dioxo-2,3-dihydrospiro[indene-1,5′-ozazolidine]-3′-yl)acetamide

To a stirring solution of N-benzyl-N—((S)-1-cyclopropylethyl)-2-(5-formyl-2′,4′-dioxo-2,3-dihydrospiro[indene-1,5′-ozazolidine]-3′-yl)acetamide SM1 (25 mg, 0.056 mmol) in 1,2-dichloroethane (3 mL) was added morpholine SM2 (19 mg, 0.224 mmol) followed by NaBH(OAc)₃ (47 mg, 0.224 mmol) and CH₃COOH(5 mg) at room temperature. The reaction mixture was stirred at room temperature for 2 hours. The reaction mixture was concentrated under reduced pressure, and the obtained residue was purified by Prep-HPLC to afford compound 120-1 (12 mg, 43%) as an off-white solid. LC-MS: m/z 518.2 [M+H]⁺ at RT 3.91 (100% purity)

### Example 121

To a stirring solution of intermediate 18 (43.3 mg, 0.1 mmol) in DCM (3 mL) was added triphosgene (12 mg, 0.04 mmol) followed by Et₃N (12 mg, 0.12 mmol) at 0° C. and the reaction mixture was stirred at room temperature for 3.5 h. Then 2 M methanamine in THF (0.5 mL, 1 mmol) was added at RT and the resulting mixture was stirred at room temperature for ¹H and concentrated under reduced pressure to obtain crude product, which was purified by Prep-HPLC to afford 121-1 (29 mg, 59%) as a yellow solid. LC-MS: m/z=490.9 [M+H]⁺. ¹H NMR (400 MHz, DMSO-d6): δ 8.71 (s, 1H), 7.56 (d, J=6.2 Hz, 1H), 7.45-7.10 (m, 7H), 6.14-6.05 (m, 1H), 4.79 (s, 1H), 4.75-4.50 (m, 1H) 4.52-4.22 (m, 2H), 3.77 (d, J=7.2 Hz, 1H), 3.17-2.96 (m, 2H), 2.68-2.63 (m, 3H), 2.61 (s, 1H), 2.49-2.43 (m, 1H), 1.23-1.12 (m, 3H), 0.97 (s, 1H), 0.50 (d, J=8.3 Hz, 1H), 0.34-0.09 (m, 3H).

### Example 122

### 5-(3-Hydroxypyrrolidin-1-yl)-2,3-dihydroinden-1-one (1)

To a stirring solution of SM1 (2.51 g, 11.9 mmol) in toluene (20 mL) was added pyrrolidin-3-ol (2.07 g, 23.8 mmol), Cs₂CO₃ (7.76 g, 23.8 mmol), Pd₂(dba)₃ (544 mg, 0.595 mmol) followed by BINAP (741 mg, 1.19 mmol) at room temperature. The reaction mixture was heated to 100° C. overnight under nitrogen atmosphere. The reaction mixture was cooled to room temperature and filtered. The filtrate was concentrated under reduced pressure to obtain crude product, which was purified by silica gel column chromatography eluting with 5% MeOH/DCM to afford compound 1 (0.718 g, 28%) as a dark solid. LC-MS: m/z=218 [M+H]⁺

### 5′-(3-Hydroxypyrrolidin-1-yl)-2′,3′-dihydrospiro[imidazolidine-4,1′-indene]-2,5-dione (2)

To a stirring solution of compound 1 (0.718 g, 3.31 mmol) in HCO₂NH₂ (6 mL) was added (NH₄)₂CO₃ (2.54 g, 26.48 mmol) followed by potassium cyanide (0.645 g, 9.93 mmol) at room temperature. The reaction mixture was heated to 150° C. for 3 hours. After consumption of the starting material (by TLC), the reaction mixture was diluted with water and extracted with EtOAc. Combined organic extracts were dried over anhydrous Na₂SO₄ and concentrated under reduced pressure to obtain crude product without further purification to afford compound 2 (0.92 g, 97%) as a dark oil. LC-MS: m/z=288 [M+H]⁺

### tert-Butyl 2-(5′-(3-hydroxypyrrolidin-1-yl)-2,5-dioxo-2′,3′-dihydrospiro[imidazolidine-4,1′-indene]-1-yl)acetate (3)

To a stirring solution of compound 2 (900 mg, 3.136 mmol) in DMF (5 mL) was added K₂CO₃ (649 mg, 4.704 mmol) followed by tert-butyl 2-bromoacetate (641.6 mg, 3.29 mmol) at room temperature and stirred at room temperature for 1 hour. The reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford compound 3 (684 mg, 54%) as a brown oil. LC-MS: m/z=402 [M+H]⁺

### 2-(5′-(3-Hydroxypyrrolidin-1-yl)-2,5-dioxo-2′,3′-dihydrospiro[imidazolidine-4,1′-indene]-1-yl)acetic acid (4)

To a stirring solution of compound 3 (684 mg, 1.706 mmol) in DCM (6 mL) was added TFA (6 mL) at room temperature and stirred at room temperature for 2 hours. After consumption of the starting material (by TLC), the reaction mixture was concentrated under reduced pressure to obtain crude compound 4 (685 mg, 99%) as a dark oil. LC-MS: m/z=346 [M+H]⁺

To a stirring solution of compound 4 (100 mg, 0.29 mmol) in DMF (1 mL) was added intermediate 24 (50.75 mg, 0.29 mmol) and DIPEA (82.3 mg, 0.638 mmol) followed by HATU (121.2 mg, 0.319 mmol) at room temperature and stirred at room temperature for 1 hour. The reaction mixture was diluted with water and extracted with EtOAc. Combined organic extracts were dried over anhydrous Na2SO4 and concentrated under reduced pressure to obtain crude product, which was purified by Prep-HPLC to afford 122-1 (36 mg, 25%) as a white solid. LC-MS: m/z=503.2 [M+H]⁺. ¹H NMR (400 MHz, DMSO-d6): δ 8.63 (d, J=16.0 Hz, 1H), 7.67-7.54 (m, 1H), 7.42-6.90 (m, 7H), 6.41 (s, 1H), 4.62 (s, 2H), 4.39 (s, 1H), 4.16 (s, 1H), 3.72 (s, 1H), 3.43-3.18 (m, 4H), 3.10-2.89 (m, 4H), 2.15 (s, 1H), 2.01 (s, 1H), 1.89 (s, 1H), 1.22-1.06 (m, 3H), 0.95 (s, 1H), 0.47 (s, 1H), 0.21 (s, 3H).

### Example 123

### (S)-methyl 5-bromo-1-isocyanato-2,3-dihydro-1H-indene-1-carboxylate (1)

To a stirring solution of intermediate 3 (1.4 g, 5.2 mmol) in DCM cooled to 0° C. and was added Et₃N (0.8 g, 7.8 mmol) followed by triphosgene (0.61 g, 2.1 mmol). The reaction mixture was stirred under N₂ for 1 hour. After consumption of the starting material (by TLC), the reaction mixture was used to the next step without any purification. LC-MS: m/z=297.12 [M+H]⁺

### (S)-methyl 1-(3-(2-(benzyl((S)-1-cyclopropylethyl)amino)-2-oxoethyl)ureido)-5-bromo-2,3-dihydro-1H-indene-1-carboxylate (2)

The mixture from the previous step was added another equal Et₃N and followed by intermediate 4 (1.2 g, 5.2 mmol) and stirred under N₂ at room temperature overnight, After consumption of the starting material (by TLC), the solvent from reaction mixture was removed under reduced pressure. Obtained residue was used to the next step without any other purification. LC-MS: m/z=528.1/530.1 [M+H]⁺

### N-benzyl-2-((S)-5′-bromo-2,5-dioxo-2′,3′-dihydrospiro[imidazolidine-4,1′-indene]-1-yl)-N—((S)-1-cyclopropylethyl)acetamide (3)

To a stirring solution of compound 2 (1.2 g, 2.7 mmol) in 30 mL THF was added 1N LiOH (8.1 mmol) and the mixture was stirred at room temperature for 3 hours, After consumption of the starting material (by TLC), the mixture was diluted with water and extracted with DCM, Combined organic extracts were dried over anhydrous Na₂SO₄ and concentrated under reduced pressure to obtain crude product, which was purified by silica gel column chromatography to afford compound 3 (1.1 g, 55%) as an off-white solid. LC-MS: m/z=496.1/498.1 [M+H]⁺

To a stirring solution of compound 3 (50 mg, 0.1 mmol) in DMF/Et₃N (6 ml, v/v=2/1) was added 2-methylbut-3-yn-2-ol (0.4 mL, 2.1 mmol) followed by Pd(PPh₃)₄ (3.5 mg, 0.07 mmol) and CuI (2.0 mg, 0.014 mmol) at room temperature. The reaction mixture was refluxing under N₂ overnight. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% MeOH/DCM to afford 123-1 (10 mg, 11%) as white solid. LC-MS: m/z=500.1 [M+H] at RT 1.58 (100% purity). ¹H NMR (400 MHz, DMSO) δ 8.83 (d, J=17.8 Hz, 1H), 7.51-7.14 (m, 8H), 4.74 (s, 1H), 4.62 (s, 1H), 4.50-4.25 (m, 1H), 4.19 (d, J=6.9 Hz, 1H), 3.71 (dd, J=9.5, 6.8 Hz, 1H), 3.02 (dd, J=13.8, 7.0 Hz, 2H), 2.62-2.52 (m, 1H), 2.31-2.12 (m, 1H), 1.45 (t, J=7.3 Hz, 6H), 1.26-1.04 (m, 3H), 0.93 (s, 1H), 0.55-0.33 (m, 1H), 0.20 (ddd, J=32.2, 18.4, 10.3 Hz, 3H).

### Example 124

### (S)—N-(3′-(2-(benzyl(1-cyclopropylethyl)amino)-2-oxoethyl)-2′,4′-dioxo-2,3-dihydrospiro[indene-1,5′-ozazolidine]-5-yl)-3-oxobutanamide

To a stirring solution of Intermediate 18 (50 mg, 0.11 mmol) and ethyl 3-oxobutanoate (30 mg, 0.22 mmol) in dry toluene (3 mL) was added NaOH (cat.) at room temperature. The reaction mixture was stirred at reflux overnight then concentrated and purified by silica gel column chromatography eluting with 100% EtOAC/Hexane to afford compound 124-1 (15 mg, 26%). TLC: 150% EtOAC/Hexane (Rf: 0.4). LC-MS: m/z=518 [M+H]⁺ (98% purity). ¹H NMR (400 MHz, DMSO-d6) δ10.26 (s, 1H), 7.77 (s, 1H), 7.48-7.10 (m, 7H), 4.79 (s, 1H), 4.70-4.60 (m, 1H), 4.50-4.30 (m, 2H), 3.82-3.75 (m, 1H), 3.22-3.01 (m, 3H), 2.70-2.62 (m, 1H), 2.21 (s, 3H), 1.24-1.12 (m, 3H), 1.05-0.90 (m, 2H), 0.55-0.40 (m, 2H), 0.35-0.11 (m, 3H).

### Example 125

### 5-morpholino-2,3-dihydrospiro[indene-1,5′-ozazolidine]-2′,4′-dione (1)

To a stirring solution of compound SM1 (140 mg, 0.11 mmol), K₃PO₄ (456 mg, 2.15 mmol), L-proline (63.2 mg, 0.55 mmol) and CuI(133 mg, 0.7 mmol) in DMSO (3 mL) was added morpholine (113 mg, 1.3 mmol) at room temperature. The reaction mixture was stirred at 90° C. overnight then quenched with H₂O and extracted with EtOAc. Combined organic extracts were dried over anhydrous Na₂SO₄ and concentrated and purified by silica gel column chromatography eluting with 50% EtOAc/Hexane to afford compound 1 (100 mg, 70%). TLC: 50% EtOAc/Hexane (R_{f}: 0.3). LC-MS: m/z 289.1 [M+H]⁺

To a stirring solution of compound 1 (100 mg, 0.35 mmol) in DMF (1 mL) was added K₂CO₃ (100 mg, 0.7 mmol) at room temperature. To this added compound intermediate-23 (100 mg, 0.35 mmol) at room temperature and the reaction mixture was stirred at room temperature for 0.5 h. The mixture was diluted with brine, extracted with EtOAc, the organic phase was washed with brine, dried over anhydrous Na₂SO₄ and concentrated and purified by silica gel column chromatography eluting with 30% EtOAC/Hexane to afford compound 125-1 (80 mg, 38%) as white solid. TLC: 50% EtOAC/Hexane (Rf: 0.5). LC-MS: m/z=504 [M+H]⁺ (98% purity). ¹H NMR (400 MHz, DMSO-d6) δ7.52-6.87 (m, 8H), 4.78 (s, 1H), 4.70-4.40 (m, 2H), 4.36-4.19 (m, 1H), 3.73 (s, 4H), 3.16 (s, 4H), 3.05-2.96 (m, 1H), 2.65-2.55 (m, 1H), 1.27-1.13 (m, 3H), 1.02-0.80 (m, 3H), 0.65-0.15 (m, 5H).

### Example 126

### N-benzyl-2-(5-(2-cyanoacetamido)-2′,4′-dioxo-2,3-dihydrospiro[indene-1,5′-ozazolidine]-3′-yl)-N—((S)-1-cyclopropylethyl)acetamide

To a stirring solution of 2-cyanoacetic acid SM1 (0.1 g, 1.18 mmol) in DCM (10 mL) was added (COCl)₂ (the excess), the mixture was stirred at 0 degree for 30 mins, and the solvent was evaporated off in vacuo. The crude dissolved with THF (5 mL) was added dropwise to a solution of 2-(5-amino-2′,4′-dioxo-2,3-dihydrospiro[indene-1,5′-ozazolidine]-3′-yl)-N-benzyl-N—((S)-1-cyclopropylethyl)acetamide SM3 (0.255 g, 0.59 mmol), TEA (0.119 g, 1.18 mmol) in THF (5 mL). The mixture was stirred for 2 hours. After consumption of SM3 (by TLC), the solvent from reaction mixture was removed under reduced pressure, the residue was diluted with brine and extracted with EtOAc. Combined organic extracts were dried over anhydrous Na₂SO₄ and concentrated under reduced pressure to obtain crude product, which was purified by Prep-HPLC [H₂O-ACN(0.05% TFA)] to afford compound 126-1 (0.11 g, 37%) as a white liquid. ¹H NMR (400 MHz, DMSO-d₆): δ 10.46 (d, J=21.2 Hz, 1H), 7.71 (d, J=6.3 Hz, 7H), 7.55-7.02 (m, 7H), 4.72-4.33 (m, 5H), 3.78 (dd, J=16.3, 9.4 Hz, 2H), 3.76-3.62 (m, 1H), 3.26-3.02 (m, 2H), 2.72-2.52 (m, 1H), 1.53-1.35 (m, 3H), 0.98-0.69 (m, 1H), 0.54-0.23 (m, 4H).

### Example 127

### (S)-methyl 5-bromo-1-isocyanato-2,3-dihydro-1H-indene-1-carboxylate (1)

To a stirring solution of intermediate 3 (1.4 g, 5.2 mmol) in DCM cooled to 0° C. and was added Et₃N (0.8 g, 7.8 mmol) followed by triphosgene (0.61 g, 2.1 mmol). The reaction mixture was stirred under N₂ for 1 hour. After consumption of the starting material (by TLC), the reaction mixture was used to the next step without any purification.

LC-MS: m/z 297.12 [M+H]⁺

### (S)-methyl 1-(3-(2-(benzyl((S)-1-cyclopropylethyl)amino)-2-oxoethyl)ureido)-5-bromo-2,3-dihydro-1H-indene-1-carboxylate (2)

The mixture was added another equal Et₃N and followed by intermediate 4 (1.2 g, 5.2 mmol) and stirred under N₂ at room temperature overnight, After consumption of the starting material (by TLC), the solvent from reaction mixture was removed under reduced pressure. Obtained residue was used to the next step without any other purification. LC-MS: m/z=528.1/530.1 [M+H]⁺

### N-benzyl-2-((S)-5′-bromo-2,5-dioxo-2′,3′-dihydrospiro[imidazolidine-4,1′-indene]-1-yl)-N—((S)-1-cyclopropylethyl)acetamide (3)

To a stirring solution of compound 2 (1.2 g, 2.7 mmol) in 30 ml THF was added 1N LiOH (8.1 mmol) and the mixture was stirred at room temperature for 3 hours, After consumption of the starting material (by TLC), the mixture was diluted with water and extracted with DCM, Combined organic extracts were dried over anhydrous Na₂SO₄ and concentrated under reduced pressure to obtain crude product, which was purified by silica gel column chromatography to afford compound 3 (1.1 g, 55%) as an off-white solid.

LC-MS: m/z=496.1/498.1 [M+H]⁺

To a stirring solution of compound 3 (60 mg, 0.12 mmol) in DMF (4 mL) was added Pd(PPh₃)₄ (12 mg, 0.01 mmol) and Zn(CN)₂ (50 mg, 0.36 mmol) and stirred at 120° C. under microwave for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product which was purified by silica gel column chromatography eluting with 5% MeOH/DCM to afford 127-1 as white solid (15 mg, 31%). LC-MS: m/z=443.1 [M+H]⁺ at RT 1.58 (100% purity). ¹H NMR (301 MHz, DMSO) δ 8.92 (d, J=13.8 Hz, 1H), 7.78 (dd, J=20.1, 12.5 Hz, 2H), 7.68-7.32 (m, 4H), 7.33-7.08 (m, 2H), 4.68 (d, J=37.7 Hz, 2H), 4.50-4.25 (m, 1H), 4.22-4.07 (m, 1H), 3.77-3.64 (m, 1H), 3.07 (dd, J=14.6, 8.0 Hz, 2H), 2.60 (dd, J=13.3, 6.7 Hz, 1H), 2.27 (dd, J=13.6, 8.3 Hz, 1H), 1.32-1.02 (m, 3H), 0.93 (s, 1H), 0.47 (s, 2H), 0.32-0.05 (m, 2H).

### Example 128

### 3-phenyldihydrofuran-2,5-dione (1)

A mixture of SM1 (5 g, 25.75 mmol) and acetyl chloride (9.535 g, 121.46) was to reflux for 2 hours. The solvent was removed under reduced pressure and the residue was purified by silica gel column chromatography eluting with 25% EtOAc/PE to afford compound 1 (4.409 g, 97%) as a white solid. ¹H NMR (301 MHz, CDCl₃) δ 7.53-7.14 (m, 5H), 4.35 (dd, J=10.3, 6.5 Hz, 1H), 3.48 (dd, J=18.9, 10.3 Hz, 1H), 3.13 (dd, J=18.9, 6.5 Hz, 1H). 3-oxo-2,3-dihydro-1H-indene-1-carboxylic acid (2):

To a well-stirred suspension of AlCl₃ (4.409 g, 25.03 mmol) in 1,2-dichloroethane (36 mL) was added dropwise a solution of compound 1 (4.409 g, 25.03 mmol) in 1,2-dichloroethane (28 mL) at 0° C. After the reaction mixture was stirred at room temperature for 40 minutes, the reaction was quenched with water (56 mL) at 0° C. and then extracted with ether (3×42 mL). Combined organic extracts were dried over anhydrous Na₂SO₄ and concentrated under reduced pressure to obtain compound 2 (3.029 g, 69%) as a brown oil. LC-MS: m/z 177 [M+H]⁺

### tert-butyl 3-oxo-2,3-dihydro-1H-indene-1-carboxylate (3)

To a stirring solution of compound 2 (0.5 g, 2.838 mmol) in THF (5 mL) was added DMAP (173 mg, 1.419 mmol) and t-butanol (2.5 mL) followed by Boc₂O (1.115 g, 5.11 mmol) at room temperature. After stirred at room temperature for 2 hours, the solvent was removed under reduced pressure and purified by silica gel column chromatography eluting with 10% EtOAc/PE to afford compound 3 (327 mg, 50%) as a off-white solid. LC-MS: m/z 233 [M+H]⁺

### tert-butyl 2,5-dioxo-2′,3′-dihydrospiro[imidazolidine-4,1′-indene]-3′-carboxylate (4)

To a stirring solution of compound 3 (160 mg, 0.690 mmol) in formamide (2 mL) was added (NH₄)₂CO₃ (662 mg, 6.9 mmol) followed by potassium cyanide (135 mg, 2.07 mmol) at room temperature in a sealed tube. The reaction mixture was heated to 100° C. overnight. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% methanol/DCM to afford compound 4 (75 mg, 36%) as a yellow solid. LC-MS: m/z 303 [M+H]⁺

### tert-butyl 1-(2-(benzyl((S)-1-cyclopropylethyl)amino)-2-oxoethyl)-2,5-dioxo-2′,3′-dihydrospiro[imidazolidine-4,1′-indene]-3′-carboxylate (5)

To a stirring solution of compound 4 (75 mg, 0.248 mmol) in DMF (2 mL) was added K₂CO₃ (69 mg, 0.496 mmol) followed by intermediate 23 (88 mg, 0.298 mmol) at room temperature and stirred at room temperature overnight. The reaction mixture was diluted with water and extracted with EtOAc. Combined organic extracts were dried over anhydrous Na₂SO₄ and concentrated under reduced pressure to obtain the crude product, which was purified by silica gel column chromatography eluting with 5% methanol/DCM to afford compound 5 (130 mg, 99%) as a yellow oil. LC-MS: m/z 518 [M+H]⁺

To a stirring solution of compound 5 (130 mg, 0.251 mmol) in DCM (1 mL) was added TFA (1 mL) at room temperature and stirred at room temperature for 1 hour. After consumption of the starting material (by TLC), the reaction mixture was concentrated under reduced pressure to obtain crude product, which was purified by prep-HPLC eluting with ACN/H₂O containing 5% TFA to afford 128-1 (1 A, 13 mg, 11%; 1B, 14 mg, 11%) as a white solid. LC-MS: m/z=462.2 [M+H]⁺ P1 at RT 4.091 (95.407% purity) P2 at room temperature 4.265 (91.541% purity). 133-1 A ¹H NMR (400 MHz, MeOD): δ 7.85-7.08 (m, 9H), 4.84-4.69 (m, 2H), 4.60-4.20 (m, 3H), 3.91-3.75 (m, 0.7H), 3.39 (d, J=8.3 Hz, 0.3H), 3.19-2.99 (m, 1H), 2.57 (ddd, J=13.8, 11.2, 8.2 Hz, 1H), 1.28 (dd, J=34.0, 6.6 Hz, 3H), 0.97 (d, J=7.9 Hz, 1H), 0.66-0.46 (m, 1H), 0.46-0.08 (m, 3H). 133-1B ¹H NMR (400 MHz, MeOD): δ 7.70-7.08 (m, 9H), 4.83-4.62 (m, 2H), 4.62-4.19 (m, 3H), 3.85-3.76 (m, 0.7H), 3.37 (s, 0.3H), 3.04-2.81 (m, 1H), 2.73-2.58 (m, 1H), 1.42-1.06 (m, 3H), 0.95 (d, J=8.0 Hz, 1H), 0.65-0.49 (m, 1H), 0.46-0.18 (m, 3H).

### Example 129

### N-benzyl-N—((S)-1-cyclopropylethyl)-2-((S)-5′-isocyanato-2,5-dioxo-2′,3′-dihydrospiro[imidazolidine-4,1′-indene]-1-yl)acetamide (1)

To a suspension of intermediate 33 (31 mg, 0.07 mmol) in DCM (5 mL) was added triphosgene (7.2 mg, 0.024 mmol) and TEA (8.5 mg, 0.084 mmol) at 0° C. under N₂, the reaction mixture was stirred at room temperature for 4 h. After consumption of the starting material (by TLC), used in next step without purification. TLC: 5% MeOH/DCM (R_{f}: 0.6)

To a solution of compound 1 (5 mL, 0.07 mmol) was added MeNH₂ (1N in THF, 0.35 mL), then stirred at room temperature under N₂ for 2 hours. After consumption of the starting material (by TLC) and concentrated under reduced pressure to obtain crude product, which was purified by Prep-HPLC (MeCN/H₂O 2:1) to afford 129-1 (20 mg, 59%) as white solid. LC-MS: m/z=490.0 [M+H]⁺ at RT 3.65 (99.32% purity). ¹H NMR (300 MHz, CD₃OD) δ 7.88-6.77 (m, 8H), 4.83-4.68 (m, 2H), 4.47 (d, J=7.1 Hz, 1H), 4.32 (s, 1H), 3.81 (dd, J=9.6, 6.9 Hz, 1H), 3.08 (s, 2H), 2.75 (d, J=8.2 Hz, 3H), 2.72-2.61 (m, 1H), 2.40-2.22 (m, 1H), 1.25 (dd, J=24.6, 6.7 Hz, 3H), 0.94 (dd, J=12.6, 8.8 Hz, 1H), 0.66-0.50 (m, 1H), 0.48-0.12 (m, 3H).

### Example 130

### 5-(2-oxa-6-azaspiro[3.3]heptan-6-yl)-2,3-dihydrospiro[indene-1,5′-ozazolidine]-2′,4′-dione (1)

To a solution of intermediate-25 (282 mg, 1.0 mmol) and SM1 (258 mg, 2.6 mmol) in DMSO (6 mL) was added K₃PO₄ (913 mg, 4.3 mmol), CuI (267 mg, 1.4 mmol) and L-proline (127 mg, 1.1 mmol) under N₂. Then stirred at 90° C. for 16 h. After consumption of the starting material (by TLC), diluted with water and extracted with EtOAc. Combined organic extracts were dried over anhydrous MgSO₄ and concentrated under reduced pressure to obtain crude product, which was purified by silica gel column chromatography eluting with 60% EtOAc/PE to afford compound 1 (150 mg, 50%) as a white solid.

To a stirring solution of compound 1 (150 mg, 0.5 mmol) and intermediate-23 (162 mg, 0.55 mmol) in DMF (5 mL) was added K₂CO₃ (138 mg, 1.0 mmol) and stirred at room temperature for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by prep-hplc to afford 130-1 (100 mg, 41%) as a white solid. LC-MS: m/z=516.2 [M+H]⁺ at RT 4.98 (99.73% purity). ¹H NMR ((400 MHz, DMSO-d6) δ 7.33 (m, 6H), 6.38 (s, 2H), 4.85-4.55 (m, 6H), 4.51-4.19 (m, 2H), 4.00 (s, 4H), 3.76 (s, 1H), 3.38 (s, 0.5H), 3.20-2.84 (m, 2H), 2.76-2.53 (m, 1.5H), 1.17 (dd, J=20.6, 5.9 Hz, 3H), 0.97 (s, 1H), 0.57-0.07 (m, 4H).

### Example 131

To a stirring solution of N-benzyl-N—((S)-1-cyclopropylethyl)-2-(5-formyl-2′,4′-dioxo-2,3-dihydrospiro[indene-1,5′-ozazolidine]-3′-yl)acetamide SM1 (45 mg, 0.1 mmol) in 1,2-dichloroethane (6 mL) was added piperidine SM2 (34 mg, 0.4 mmol) followed by NaBH(OAc)₃ (85 mg, 0.4 mmol) and CH₃COOH (5 mg) at room temperature. The reaction mixture was stirred at room temperature for 16 h. The reaction mixture was concentrated under reduced pressure, and the obtained residue was purified by Prep-HPLC to afford compound 131-1 (31 mg, 60%) as an off-white solid. LC-MS: m/z=516.2 [M+H]⁺ at RT 4.14 (99.89% purity). ¹H NMR (300 MHz, DMSO-d6) δ 9.48 (s, 1H), 7.58 (dd, J=12.5, 6.7 Hz, 2H), 7.50-6.89 (m, 6H), 4.80 (s, 1H), 4.58 (ddd, J=48.6, 16.6, 9.5 Hz, 2H), 4.35 (dd, J=12.6, 6.7 Hz, 3H), 3.81-3.72 (m, 0.5H), 3.45-3.24 (m, 2.5H), 3.22-3.02 (m, 2H), 2.88 (d, J=9.7 Hz, 2H), 2.63 (ddd, J=12.7, 12.0, 6.9 Hz, 2H), 1.80 (d, J=12.5 Hz, 2H), 1.62 (d, J=16.3 Hz, 3H), 1.37 (s, 1H), 1.18 (dd, J=20.6, 6.5 Hz, 3H), 0.99 (s, 1H), 0.60-0.09 (m, 4H).

### Example 132

### N-benzyl-N—((S)-1-cyclopropylethyl)-2-(5-formyl-2′,4′-dioxo-2,3-dihydrospiro[indene-1,5′-ozazolidine]-3′-yl)acetamide (1)

To a stirring suspension of formic acid (6 mL) and Raney-Ni (40 mg) was added N-benzyl-2-(5-cyano-2′,4′-dioxo-2,3-dihydrospiro[indene-1,5′-ozazolidine]-3′-yl)-N-((S)-1-cyclopropylethyl)acetamide SM (400 mg, 0.9 mmol) at 100° C. and kept at 100° C. for 30 min. After consumption of the starting material (by TLC), the reaction mixture was filtered. The filtrate was concentrated under reduced pressure to obtain crude product, which was purified by silica gel column chromatography eluting with 50% EtOAc/PE to afford compound 1 (175 mg, 43%) as an off-white solid. LC-MS: m/z=447.2 [M+H]⁺

### tert-butyl 4-((3′-(2-(benzyl((S)-1-cyclopropylethyl)amino)-2-oxoethyl)-2′,4′-dioxo-2,3-dihydrospiro[indene-1,5′-ozazolidine]-5-yl)methyl)piperazine-1-carboxylate(2)

To a stirring solution of compound 1 (50 mg, 0.112 mmol) in 1,2-dichloroethane (6 mL) was added tert-butyl piperazine-1-carboxylate (84 mg, 0.45 mmol) followed by NaBH(OAc)₃ (95 mg, 0.45 mmol) and CH₃COOH(6 mg) at room temperature. The reaction mixture was stirred at room temperature for 16 h. The reaction mixture was concentrated under reduced pressure, and the obtained residue was purified by silica gel column chromatography eluting with 10% CH₃OH/DCM to afford compound 2 (51 mg, 73%) as an off-white solid. LC-MS: m/z=617.2 [M+H]⁺

To a stirring solution of compound 2 (51 mg, 0.083 mmol) in DCM (2.5 mL) was added TFA (0.5 mL) at room temperature. The reaction mixture was stirred at room temperature for 30 min. After consumption of the starting material (by TLC), the reaction mixture was concentrated under reduced pressure to obtain crude product, which was purified by Prep-HPLC to afford compound 132-1 (30 mg, 58%) as a white solid. LC-MS: m/z=517.2 [M+H]⁺ at RT 3.82 (100% purity). ¹H NMR (300 MHz, DMSO-d6) δ 8.91 (s, 1H), 7.59-7.50 (m, 1H), 7.47 (s, 1H), 7.43-7.07 (m, 6H), 4.79 (s, 1H), 4.71-4.59 (m, 1H), 4.56-4.24 (m, 2H), 4.00 (s, 2H), 3.80-3.72 (m, 0.5H), 3.44-3.36 (m, 0.5H), 3.33-2.75 (m, 10H), 2.72-2.51 (m, 3H), 1.17 (dd, J=20.9, 6.6 Hz, 3H), 1.03-0.91 (m, 1H), 0.58-0.12 (m, 4H).

### Example 133

### N-benzyl-N-((S)-1-cyclopropylethyl)-2-(5-((4-hydroxypiperidin-1-yl)methyl)-2′,4′-dioxo-2,3-dihydrospiro[indene-1,5′-ozazolidine]-3′-yl)acetamide

To a stirring solution of N-benzyl-N-((S)-1-cyclopropylethyl)-2-(5-formyl-2′,4′-dioxo-2,3-dihydrospiro[indene-1,5′-ozazolidine]-3′-yl)acetamide SM1 (45 mg, 0.1 mmol) in 1,2-dichloroethane (3 mL) was added piperidin-4-ol SM2 (41 mg, 0.4 mmol) followed by K₂CO₃ (85 mg, 0.4 mmol) and CH₃COOH (5 mg) at room temperature. The reaction mixture was stirred at room temperature for 16 h. The reaction mixture was concentrated under reduced pressure, and the obtained residue was purified by Prep-HPLC to afford compound 133-1 (13 mg, 24%) as an off-white solid. LC-MS: m/z=532.2 [M+H]⁺ at RT 3.84 (100% purity). ¹H NMR (400 MHz, DMSO-d6) δ 9.38 (s, 1H), 7.59 (dd, J=18.3, 10.8 Hz, 2H), 7.53-6.90 (m, 6H), 4.80 (s, 1H), 4.66 (dd, J=15.3, 9.2 Hz, 1H), 4.53 (d, J=12.0 Hz, 1H), 4.43-4.26 (m, 3H), 3.93 (s, 1H), 3.81-3.74 (d, J=9.3 Hz, 0.5H), 3.65-3.57 (m, 0.5H), 3.49-2.83 (m, 7H), 2.79-2.52 (m, 2H), 1.96 (d, J=13.1 Hz, 1H), 1.76 (s, 2H), 1.54 (d, J=10.1 Hz, 1H), 1.18 (dd, J=27.3, 6.0 Hz, 3H), 0.99 (s, 1H), 0.56-0.11 (m, 4H).

### Example 134

### (S)-2-amino-N-(3′-(2-(benzyl(1-cyclopropylethyl)amino)-2-oxoethyl)-2′,4′-dioxo-2,3-dihydrospiro[indene-1,5′-ozazolidine]-5-yl)-2-methylpropanamide

To a stirring solution of compound intermediate-36 (22 mg, 0.14 mmol) in Py (0.5 mL) and DCM (0.5 ml) was added compound intermediate-31 (30 mg, 0.07 mmol) at room temperature and the reaction mixture was stirred at room temperature overnight. The mixture was then concentrated and purified with Pre-TLC(10% MeOH/DCM) to afford compound 134-1 (25 mg, 69%) as white solid. TLC: 10% MeOH/DCM (Rf: 0.2). LC-MS: m/z=519 [M+H]⁺ (98% purity). ¹H NMR (400 MHz, CD₃OD-d4) δ 7.74 (s, 1H), 7.56-7.23 (m, 7H), 4.84-4.74 (m, 2H), 4.68-4.55 (m, 1H), 4.45 (t, J=4.6 Hz, 1H), 3.90-3.82 (m, 1H), 3.20-2.12 (m, 1H), 2.84-2.76 (m, 1H), 2.63-2.55 (m, 1H), 1.70 (d, J=2.5 Hz, 6H), 1.28 (dd, J=21.8, 6.7 Hz, 3H), 1.03-0.9 (m, 2H), 0.68-0.53 (m, 2H), 0.40-0.25 (s, 3H).

### Example 135

### tert-butyl piperazine-1-carboxylate (1)

To a stirring solution of compound SM1 (4 g, 46.5 mmol) and DIPEA (6 g, 46.5 mmol) in dry DCM (500 mL) was added a solution of di-tert-butyl dicarbonate (5.07 g, 23.2 mmol) in 50 mL of dry DCM by a syringe pump in a period of 20 h at room temperature. The reaction mixture was then filtered and concentrated to afford compound 1 (4.2 g, 48%). TLC: 5% MeOH/DCM (R_{f}: 0.3) tert-butyl 4-(2′,4′-dioxo-2,3-dihydrospiro[indene-1,5′-ozazolidine]-5-yl)piperazine-1-carboxylate (3):

To a stirring solution of compound 1 (400 mg, 2.12 mmol), K₃PO₄ (966 mg, 4.56 mmol), L-proline (134 mg, 1.06 mmol) and CuI (283 mg, 1.5 mmol) in DMSO (3 mL) was added compound 2 (300 mg, 1.06 mmol) at room temperature. The reaction mixture was stirred at 90° C. overnight then quenched with H₂O and extracted with EtOAc. Combined organic extracts were dried over anhydrous Na₂SO₄ and concentrated and purified by silica gel column chromatography eluting with 50% EtOAc/Hexane to afford compound 3 (100 mg, 70%). TLC: 50% EtOAc/Hexane (R_{f}: 0.2)

### (S)-tert-butyl 4-(3′-(2-(benzyl(1-cyclopropylethyl)amino)-2-oxoethyl)-2′,4′-dioxo-2,3-dihydrospiro[indene-1,5′-ozazolidine]-5-yl)piperazine-1-carboxylate (4)

To a stirring solution of compound 3 (80 mg, 0.21 mmol) in DMF (1 mL) was added K₂CO₃ (60 mg, 0.42 mmol) at room temperature. To this added compound Intermediate-23 (76 mg, 0.21 mmol) at room temperature and the reaction mixture was stirred at room temperature for 0.5 h. The mixture was diluted with brine, extracted with EtOAc, the organic phase was washed with brine, dried over anhydrous Na₂SO₄ and concentrated and purified by silica gel column chromatography eluting with 40% EtOAC/Hexane to afford compound 4 (25 mg, 20%). TLC: 50% EtOAc/Hexane (R_{f}: 0.5)

A solution of compound 3 (25 mg, 0.04 mmol) in DCM (1 mL) was added 0.2 ml of TFA, the reaction mixture was stirred at room temperature for ¹H then concentrated and purified with Pre-TLC (10% MeOH/DCM) to afford compound 135-1 (8 mg, 26%) as white solid. TLC: 10% MeOH/DCM (Rf: 0.3). LC-MS: m/z=503 [M+H]⁺ (98% purity). ¹H NMR (400 MHz, CD₃OD-d4) 67.46-7.36 (m, 3H), 7.32-7.18 (m, 3H), 7.09-6.91 (m, 2H), 4.83-4.75 (m, 2H), 4.68-4.40 (m, 2H), 3.50-3.42 (m, 4H), 3.40-3.32 (m, 4H), 3.18-3.03 (m, 2H), 2.82-2.72 (m, 1H), 2.58-2.51 (m, 1H), 1.31 (d, J=6.6 Hz, 1H), 1.24 (d, J=6.8 Hz, 2H), 1.02-0.94 (s, 1H), 0.66-0.50 (m, 2H), 0.40-0.20 (m, 3H).

### Example 136

### 5′-(3-Hydroxyazetidin-1-yl)-2′,3′-dihydrospiro[imidazolidine-4,1′-indene]-2,5-dione (1)

To a stirring solution of Intermediate 10 (281 mg, 1 mmol) in DMSO (2 mL) was added azetidin-3-ol hydrochloride (219 mg, 2 mmol), K₃PO₄ (913 mg, 4.3 mmol), L-proline (126.6 mg, 1.1 mmol) followed by CuI (267 mg, 1.4 mmol) at room temperature. The reaction mixture was heated to 90° C. overnight under nitrogen atmosphere. The reaction mixture was cooled to room temperature and diluted by water and extracted with EtOAc. Combined organic extracts were dried over anhydrous Na₂SO₄ and concentrated under reduced pressure to obtain crude product, which was purified by silica gel column chromatography eluting with 10% MeOH/DCM to afford compound 1 (45 mg, 16%) as a yellow oil. LC-MS: m/z 274 [M+H]⁺

To a stirring solution of compound 2 (45 mg, 0.165 mmol) in DMF (1 mL) was added K₂CO₃ (34 mg, 0.248 mmol) followed by intermediate 23 (49 mg, 0.165 mmol) at room temperature and stirred at room temperature for 2 hours. The reaction mixture was diluted with water and extracted with EtOAc. Combined organic extracts were dried over anhydrous Na₂SO₄ and concentrated under reduced pressure to obtain crude product, which was purified by Prep-HPLC to afford 136-1 (6 mg, 7%) as a yellow solid. LC-MS: m/z=489.3 [M+H]⁺. ¹H NMR (400 MHz, DMSO-d6): δ 8.64 (d, J=16.1 Hz, 1H), 7.38 (q, J=7.8 Hz, 2H), 7.33-7.12 (m, 3H), 7.12-6.84 (m, 2H), 6.53 (s, 1H), 6.36-6.26 (m, 1H), 4.74 (s, 1H), 4.62 (s, 1H) 4.55 (s, 1H), 4.46-4.35 (m, 1H), 4.25 (dd, J=22.2, 5.9 Hz, 1H), 4.18-4.03 (m, 2H), 3.71 (s, 1H), 3.47 (d, J=6.0 Hz, 1H), 3.37 (s, 1H), 2.93 (s, 2H), 2.46 (d, J=6.1 Hz, 1H), 2.13 (d, J=12.9 Hz, 1H), 1.22-1.06 (m, 3H), 0.95 (s, 1H), 0.47 (s, 1H), 0.40-0.15 (m, 3H).

### Example 137

### 2-cyanoacetyl chloride (3)

To a solution of 2-cyanoacetic acid 1 (85 mg, 1 mmol) in THF (10 mL) was added oxalyl dichloride (500 mg, 4 mmol) and DMF (7 mg, 0.1 mmol) at 0° C. under N₂, the reaction mixture was stirred at room temperature for 1 hour. Concentrated under reduced pressure to obtain crude compound 3 (0.13 g, 120%)

To a solution of 2-cyanoacetyl chloride 3 (130 mg, 1 mmol) in THF (10 ml) was added intermediate 33 (31 mg, 0.07 mmol), then stirred at room temperature under N₂ for 4 h. After consumption of the starting material (by TLC) diluted with water and extracted with EtOAc. Combined organic extracts were dried over anhydrous MgSO₄ and concentrated under reduced pressure to obtain crude product, which was purified by Prep-HPLC (MeCN/H₂O 2:1) to afford 137-1 (30 mg, 85%) as brown solid. LC-MS: m/z=500.3 [M+H]⁺ at RT 4.16 (95.45% purity). ¹H NMR (300 MHz, CD₃OD) δ 7.62 (s, 1H), 7.54-6.93 (m, 7H), 4.86-4.65 (m, 2H), 4.58-4.31 (m, 2H), 3.82-3.72 (m, 2H), 3.64-3.42 (m, 1H), 3.12 (d, J=2.9 Hz, 2H), 2.79-2.63 (m, 1H), 2.32 (dd, J=13.5, 5.9 Hz, 1H), 1.25 (dd, J=25.0, 6.7 Hz, 3H), 1.04-0.84 (m, 1H), 0.64-0.47 (m, 1H), 0.31 (ddd, J=14.8, 7.8, 3.9 Hz, 3H).

### Example 138

### 2,5-dioxo-2′,3′-dihydrospiro[imidazolidine-4,1′-indene]-3′-carboxylic acid (1)

To a stirring solution of compound SM1 (500 mg, 1.66 mmol) in DCM (4 mL) was added TFA (4 mL) at room temperature and stirred at room temperature for 1 hour. After consumption of the starting material (by TLC), the reaction mixture was concentrated under reduced pressure to obtain crude compound 1 (400 mg, 99%) as a white solid. LC-MS: m/z 247 [M+H]⁺

### 2,5-dioxo-2′,3′-dihydrospiro[imidazolidine-4,1′-indene]-3′-carboxamide (2)

To a stirring solution of compound 1 (300 mg, 1.219 mmol) in DCM (8 mL) was added DMF (3 drops) followed by oxalyl chloride (186 mg, 1.463 mmol) at room temperature and stirred at room temperature for 1 hour. NH₃ gas was bubbled into the reaction mixture and then the resulting mixture was stirred for another 1 hour. The solvent was removed under reduced pressure and purified by silica gel column chromatography eluting with 5%-10% methanol in DCM to afford compound 2 (88 mg, 30%) as a white solid. LC-MS: m/z=246 [M+H]⁺

To a stirring solution of compound 2 (88 mg, 0.359 mmol) in DMF (3 mL) was added K₂CO₃ (99 mg, 0.718 mmol) followed by intermediate 23 (106 mg, 0.359 mmol) at room temperature and stirred at room temperature overnight. The reaction mixture was diluted with water and extracted with EtOAc. Combined organic extracts were dried over anhydrous Na₂SO₄ and concentrated under reduced pressure to obtain the crude product, which was purified by silica gel column chromatography eluting with 5% methanol/DCM to afford 138-1 (124 mg, 75%) as a white solid. LC-MS: m/z=461.0 [M+H]⁺ at RT 3.954 (98.7% purity). ¹H NMR (400 MHz, DMSO-d6): δ 8.80-8.61 (m, 1H), 7.91 (s, 1H), 7.52-7.04 (m, 9H), 4.85-4.01 (m, 5H), 3.84-3.62 (m, 0.6H), 3.33-3.23 (m, 0.4H), 2.56 (dd, J=11.8, 6.6 Hz, 2H), 1.25-1.01 (m, 3H), 0.93 (s, 1H), 0.53-0.03 (m, 4H).

### Example 139

To a stirring solution of 138-1 (90 mg, 0.953 mmol) in ACN (9 mL) was added Et₃N (450 mg, 4.455 mmol) followed by 2,2,2-trifluoroacetic anhydride (540 mg, 2.574 mmol) at room temperature and stirred at room temperature for 30 minutes. The solvent was removed under reduced pressure and purified by prep-HPLC eluting with ACN/H₂O containing 5% TFA to afford 139-1 (30 mg, 35%) as a white solid. LC-MS: m/z=443.1 [M+H]⁺ at RT 4.586 (96.146% purity). ¹H NMR (400 MHz, DMSO): δ 8.93 (d, J=18.1 Hz, 1H), 7.72-7.06 (m, 9H), 4.83-4.09 (m, 5H), 3.70 (dd, J=15.9, 6.5 Hz, 0.6H), 3.34 (s, 0.4H), 3.16-2.98 (m, 0.8H), 2.82-2.58 (m, 0.6H), 2.49-2.37 (m, 0.6H), 1.36-0.99 (m, 3H), 0.93 (s, 1H), 0.57-0.05 (m, 4H).

### Example 140

### (E)-N-(1-(2-(benzyl((S)-1-cyclopropylethyl)amino)-2-oxoethyl)-2,5-dioxo-2′,3′-dihydrospiro[imidazolidine-4,1′-indene]-5′-ylcarbamoyl)-3-methoxyacrylamide (2)

To a stirring solution of 2-(5′-amino-2,5-dioxo-2′,3′-dihydrospiro[imidazolidine-4,1′-indene]-1-yl)-N-benzyl-N—((S)-1-cyclopropylethyl)acetamide Intermediate 33 (100 mg, 0.23 mmol) in THF (1 mL), was added 3-methoxyacryloyl isocyanate compound 1 (58.4 mg, 0.46 mmol) at room temperature and stirred at room temperature for overnight. The reaction mixture was heated to reflux for 15 min. After consumption of the starting material (by TLC), The reaction mixture was diluted with water and extracted with EA. Combined organic extracts were dried over anhydrous Na₂SO₄ and concentrated under reduced pressure to obtain crude product, which was purified by silica gel column chromatography eluting with 50% EA/PE to afford compound 2 (30 mg, 24.8%). TLC: 50% EA/PE (R_{f}: 0.4). LC-MS: m/z 560 [M+H]⁺

To a stirring solution of compound 2 (30 mg, 0.05 mmol) in EtOH (10 mL) was added 1N H₂SO₄ (10 mL) at room temperature and stirred at reflux for 3 hours. The reaction mixture was diluted with water and extracted with EA. Combined organic extracts were dried over anhydrous Na₂SO₄ and concentrated under reduced pressure to obtain crude product, which was purified by silica gel column chromatography eluting with 30% EA/PE to afford 140-1 (10 mg, 35.7%). LC-MS: m/z=528 [M+H]⁺ at RT 1.50 (100% purity). ¹H NMR (300 MHz, DMSO-d6) δ 11.46 (s, 1H), 8.88 (d, J=13.7 Hz, 1H), 7.70 (dd, J=8.2, 2.6 Hz, 2H), 7.50-7.11 (m, 6H), 5.66 (dd, J=7.9, 2.5 Hz, 1H), 4.81-4.14 (m, 5H), 3.83-3.63 (m, 1H), 3.07 (d, J=4.5 Hz, 2H), 2.72-2.54 (m, 1H), 2.35-2.13 (m, 1H), 1.73-1.55 (m, 1H), 1.45-1.31 (m, 1H), 1.25-1.02 (m, 3H), 0.91 (t, J=7.4 Hz, 2H), 0.58-0.07 (m, 4H).

### Example 141

### N-benzyl-N—((S)-1-cyclopropylethyl)-2-(2′,4′-dioxo-5-(4,4,5,5-tetramethyl-1,3,2-dioxaborolan-2-yl)-2,3-dihydrospiro[indene-1,5′-ozazolidine]-3′-yl)acetamide (1)

To a stirring solution of SM (1.4 g, 4.2 mmol) and Intermediate-23 (1.24 g, 4.2 mmol) in DMF (10 mL) was added K₂CO₃ (1.16 g, 8.4 mmol) and stirred at room temperature for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 50% EtOAc/PE to afford compound 1 (2.1 g, 92%) as a brown solid. LC-MS: m/z=545.2 [M+H]⁺

To a stirring solution of compound 1 (2.1 g, 3.85 mmol) in CH₃OH (25 mL) was added 30% H₂O₂ (2 mL) at room temperature. The reaction mixture was stirred at room temperature for 2 hours under N₂. The reaction mixture was concentrated under reduced pressure, and the obtained residue was purified by silica gel column chromatography eluting with 50% EtOAc/PE to afford compound 141-1 (910 mg, 54%) as a white solid. LC-MS: m/z=435.1 [M+H]⁺ at RT 4.68 (99.16% purity). ¹H NMR (300 MHz, DMSO-d6) δ 9.82 (s, 1H), 7.44-7.11 (m, 6H), 6.70 (dd, J=13.9, 5.7 Hz, 2H), 4.78 (s, 1H), 4.67-4.57 (m, 1H), 4.51-4.39 (m, 1H), 4.32 (dd, J=5.8, 4.3 Hz, 1H), 3.75 (dd, J=9.2, 7.1 Hz, 0.5H), 3.41-3.34 (m, 0.5H), 3.10-2.93 (m, 2H), 2.65-2.56 (m, 1H), 2.48-2.36 (m, 1H), 1.17 (ddd, J=8.6, 6.6, 2.6 Hz, 3H), 1.02-0.93 (m, 1H), 0.52-0.17 (m, 4H).

### Example 142

### Methyl 2-(3′-(2-(benzyl((S)-1-cyclopropylethyl)amino)-2-oxoethyl)-2′,4′-dioxo-2,3-dihydrospiro[indene-1,5′-ozazolidine]-5-yloxy)acetate

To a stirring solution of 141-1 (20 mg, 0.046 mmol) in CH₃CN (5 mL) was added compound 1 (10.5 mg, 0.069 mmol), followed by K₂CO₃ (12.7 mg, 0.092 mmol) at room temperature and stirred at room temperature for 3 hours. The reaction mixture was diluted with water and extracted with EA. Combined organic extracts were dried over anhydrous Na₂SO₄ and concentrated under reduced pressure to obtain crude product, which was purified by silica gel column chromatography eluting with 50% EA/PE to afford 142-1 (15 mg, 65.2%). TLC: 50% EtOAc/Hexane (R_{f}: 0.4). ¹H NMR (300 MHz, DMSO-d6) δ 7.82-7.60 (m, 1H), 7.47-6.81 (m, 7H), 4.97-4.13 (m, 7H), 3.70 (d, J=2.8 Hz, 3H), 3.24-2.58 (m, 3H), 1.76-1.56 (m, 1H), 1.44-1.29 (m, 1H), 1.25-1.09 (m, 3H), 0.91 (t, J=7.4 Hz, 2H), 0.60-0.09 (m, 4H). LC-MS: m/z 507 [M+H]⁺

### Example 143

To a stirring solution of compound 141-1 (109 mg, 0.25 mmol) and 3-bromoprop-1-yne (60 mg, 0.5 mmol) in CH₃CN (4 mL) was added K₂CO₃ (69 g, 0.5 mmol) and stirred at room temperature for 2 days. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 50% EtOAc/PE to afford compound 143-1 (42 mg, 36%) as a white solid. LC-MS: m/z 473.2 [M+H]⁺ at RT 5.40 (95.49% purity). ¹H NMR (300 MHz, CD₃OD) δ 7.64-7.11 (m, 6H), 7.03-6.85 (m, 2H), 4.90-4.87 (m, 2H), 4.77 (dd, J=8.9, 6.6 Hz, 2H), 4.58 (dd, J=16.2, 9.8 Hz, 1H), 4.43 (t, J=4.2 Hz, 1H), 3.87-3.79 (m, 0.5H), 3.39-3.32 (s, 0.5H), 3.11 (ddd, J=16.5, 14.9, 5.9 Hz, 2H), 3.00-2.93 (m, 1H), 2.84-2.71 (m, 1H), 2.63-2.50 (m, 1H), 1.27 (dd, J=22.2, 6.8 Hz, 3H), 1.04-0.91 (m, 1H), 0.60-0.25 (m, 4H).

### Example 144

To a solution of SM 1 (10 mg, 0.10 mmol) in THF (5 mL) was added intermediate 35 (50 mg, 0.12 mmol) and DIPEA (22 mg, 0.17 mmol), then stirred at room temperature under N₂ for 2 hours. After consumption of the starting material (by TLC) diluted with water and extracted with EtOAc. Combined organic extracts were dried over anhydrous MgSO₄ and concentrated under reduced pressure to obtain crude product, which was purified by Prep-HPLC (MeCN/H₂O 2:1) to afford 144-1 (35 mg, 63%) as white solid. LC-MS: m/z=487.2 [M+H]⁺ at RT 4.22 (93.47% purity). ¹H NMR (400 MHz, DMSO-d6) δ 10.22 (s, 1H), 8.76 (d, J=12.7 Hz, 1H), 7.72 (s, 1H), 7.40 (m, 3H), 7.32-7.09 (m, 4H), 6.51-6.35 (m, 1H), 6.31-6.17 (m, 1H), 5.84-5.67 (m, 1H), 4.68 (d, J=37.5 Hz, 2H), 4.27 (m, 2H), 3.71 (m, 0.5H), 3.36 (m, 0.5H), 3.01 (d, J=4.8 Hz, 2H), 2.55 m, 1H), 2.20 (m, 1H), 1.21-1.04 (m, 3H), 0.91 (m, 1H), 0.56-0.09 (m, 4H).

### Example 145

### N-benzyl-N—((S)-1-cyclopropylethyl)-2-(5′-formyl-2,5-dioxo-2′,3′-dihydrospiro[imidazolidine-4,1′-indene]-1-yl)acetamide (1)

To a stirring solution of intermediate 27 (170 mg, 0.342 mmol) in THF (5 mL) was added n-BuLi (2.4 M, 0.428 mL, 1.027 mmol) at −78° C. The reaction mixture was stirred −78° C. under N₂ for 20 minutes. Then DMF (50 mg, 0.684 mmol) was added and the resulting mixture was stirred at −78° C. for 1 hour. After consumption of the starting material (by TLC), the reaction was quenched and extracted with EtOAc. Combined organic extracts were dried over anhydrous Na₂SO₄ and concentrated under reduced pressure to obtain crude product, which was purified by silica gel column chromatography eluting with 5% methanol/DCM to afford compound 1 (92 mg, 60%) as a white solid. LC-MS: m/z=446 [M+H]⁺

To a stirring solution of compound 1 (40 mg, 0.09 mmol) in dioxane (0.225 mL) was added water (0.225 mL) and DABCO (10 mg, 0.09 mmol) followed by acrylonitrile (1 mL) at room temperature and stirred at room temperature for 48 hours. The reaction mixture was diluted with water and extracted with EtOAc. Combined organic extracts were dried over anhydrous Na₂SO₄ and concentrated under reduced pressure to obtain crude product, which was purified by prep-HPLC eluting with ACN/H₂O containing 5% TFA to afford 145-1 (12 mg, 27%) as a white solid. LC-MS: m/z=499.2 [M+H]⁺ at RT 4.324 (99.788% purity). ¹H NMR (400 MHz, CD3OD): δ 7.65-7.10 (m, 8H), 6.19 (s, 1H), 6.05 (s, 1H), 5.33 (s, 1H), 4.83-4.22 (m, 4H), 3.83 (ddd, J=9.8, 6.7, 3.4 Hz, 0.6H), 3.38 (d, J=7.0 Hz, 0.4H), 3.18 (dd, J=15.0, 7.8 Hz, 2H), 2.82-2.67 (m, 1H), 2.37 (dt, J=15.9, 8.6 Hz, 1H), 1.35-1.17 (m, 3H), 1.02-0.87 (m, 1H), 0.67-0.50 (m, 1H), 0.45-0.18 (m, 3H).

### Example 146

### (S)-2-(tert-Butoxycarbonylamino)-3-methylbutanoic acid (1)

To a stirring solution of SM1 (400 mg, 3.42 mmol) in THF (4 mL), H₂O (4 mL) was added (Boc)₂O (1.1 g, 5.13 mmol) followed by NaOH (342 mg, 8.55 mmol) at room temperature. The reaction mixture was stirred at room temperature overnight. After consumption of the starting material (by TLC), the reaction mixture was neutralized by 2N HCl, extracted with EtOAc, washed by brine. Combined organic extracts were dried over anhydrous Na₂SO₄ and concentrated under reduced pressure to obtain crude compound 1 (730 mg, 98%) as a clear oil. LC-MS: m/z=216 [M+H]⁺

### (S)-2-(tert-Butoxycarbonylamino)-3-methylbutanoic (isobutyl carbonic) anhydride (2)

To a stirring solution of compound 1 (25 mg, 0.115 mmol) in THF (3 mL) was added NMM (23.2 mg, 0.23 mmol) followed by isobutyl carbonochloridate (15.6 mg, 0.115 mmol) −15° C. and stirred at −15° C. for 20 min. After consumption of the starting material (by TLC), the reaction mixture was concentrated to afford compound 2 (36 mg, 99%) as a white solid. LC-MS: m/z=318 [M+H]⁺

### tert-Butyl (S)-1-(3′-(2-(benzyl((S)-1-cyclopropylethyl)amino)-2-oxoethyl)-2′,4′-dioxo-2,3-dihydrospiro[indene-1,5′-ozazolidine]-5-ylamino)-3-methyl-1-oxobutan-2-ylcarbamate (3)

To a stirring solution of compound 2 (36 mg, 0.115 mmol) in THF (3 mL) was added Intermediate 18 (50 mg, 0.115 mmol) at room temperature and the mixture was stirred at room temperature overnight. After consumption of the starting material (by TLC), the reaction mixture was concentrated under reduced pressure and the residue was dissolved in EtOAc, washed by 10% Na₂CO₃ solution, 0.1 M HCl, brine, combined the organic layers were dried over anhydrous Na₂SO₄ and concentrated under reduced pressure to obtain crude compound 3 (60 mg, 98%) as a yellow oil. LC-MS: m/z=533 [M+H-Boc]⁺

To a stirring solution of compound 3 (40 mg, 0.0633 mmol) in DCM (1 mL) was added TFA (1 mL) at room temperature and stirred at room temperature for 1 hour. After consumption of the starting material (by TLC), the reaction mixture was concentrated under reduced pressure to obtain crude product, which was purified by Prep-HPLC to afford 146-1 (12 mg, 36%) as a white solid. LC-MS: m/z=533.2 [M+H]⁺. ¹H NMR (400 MHz, DMSO-d6): δ 10.61 (s, 1H), 8.22 (s, 2H), 7.77-7.70 (m, 1H), 7.52-7.17 (m, 7H), 4.80 (s, 1H), 4.70-4.61 (m, 1H), 4.47 (dd, J=31.5, 14.7 Hz, 1H), 4.35 (dd, J=9.8, 5.4 Hz, 1H), 3.75 (s, 2H), 3.20 (dd, J=15.9, 7.5 Hz, 2H), 3.10 (s, 1H), 2.18 (s, 1H), 1.25-1.12 (m, 3H), 0.99 (td, J=6.8, 3.0 Hz, 6H), 0.85 (d, J=13.2 Hz, 2H), 0.45 (m, 2H), 0.29-0.15 (m, 2H).

### Example 147

### (S)-2-(tert-butoxycarbonylamino)propanoic acid (2)

To a stirring solution of (S)-2-aminopropanoic acid SM1 (0.2 g, 2.25 mmol) in THF (10 mL) was added (Boc)₂O (0.58 g, 2.7 mmol) and 10% NaOH aq (10 mL), the mixture was stirred at room temperature for 30 mins. The solvent was removed out in vacuo, the residue was diluted with brine and extracted with EtOAc. Combined organic extracts were dried over anhydrous Na₂SO₄ and concentrated under reduced pressure to obtain crude product as an off-white solid, which was used directly for subsequent step. ¹H NMR (300 MHz, DMSO-d₆): δ 12.44 (s, 1H), 7.13 (s, 1H), 3.94 (m, 1H), 1.42 (s, 9H), 1.25 (d, J=3.0 Hz, 3H).

### tert-butyl (2S)-1-(3′-(2-(benzyl((S)-1-cyclopropylethyl)amino)-2-oxoethyl)-2′,4′-dioxo-2,3-dihydrospiro[indene-1,5′-ozazolidine]-5-ylamino)-1-oxopropan-2-ylcarbamate (5)

To a cold solution (−15° C.) of SM2 (22 mg, 0.115 mmol), and 4-methyl morpholine in dry THF (5 mL), was added dropwise isobutyl chloroformate SM3 (16 mg, 0.115 mmol) in dry THF (2 mL) over 15 mins. After the mixture was stirred for another 15 mins, intermediate 18 (50 mg, 0.115 mmol) was added. Then the mixture was allowed to slowly warm to room temperature and stirred overnight. After the evaporation of the solvent in vacuo, the residue was extracted with EA (20 mL), washed with brine (15 mL), dried over MgSO₄. The concentrated organic phase crude was purified by silica gel column chromatography to afford compound 5 (0.03 g, 43%) as an off-white solid. TLC: 50% PE/EA. LC-MS: m/z=605 [M+H]⁺

### (2S)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)propanamide

A solution of SM5 (0.03 g, 0.05 mmol), TFA (2 mL) and DCM (2 mL) was stirred for 2 hours. After consumption of the starting material (by TLC), and the solvent was evaporated off in vacuo. The crude was purified by Prep-HPLC [H₂O-ACN(0.05% TFA)] to afford compound 147-1 (18 mg, 72%) as a white liquid. ¹H NMR (400 MHz, DMSO-d₆): δ 10.68 (d, J=4.9 Hz, 1H), 8.27 (s, 2H), 7.72 (d, J=6.4 Hz, 1H), 7.51-7.23 (m, 7H), 4.80-4.37 (m, 4H), 4.03 (d, J=7.0 Hz, 1H), 3.78 (d, J=6.9 Hz, 1H), 3.66-3.28 (m, 1H), 3.23-3.05 (m, 2H), 2.67 (dd, J=14.6, 6.7 Hz, 1H), 1.46 (dd, J=6.9, 3.0 Hz, 3H), 1.18 (ddd, J=9.5, 8.8, 4.1 Hz, 3H), 1.01-0.93 (m, 1H), 0.53-0.18 (m, 4H). LC-MS: m/z=505.2 [M+H]⁺ (99.31% purity, 214 nm)

### Example 148

### tert-butyl 2-(2,5-dioxo-5′-(4,4,5,5-tetramethyl-1,3,2-dioxaborolan-2-yl)-2′,3′-dihydrospiro[imidazolidine-4,1′-indene]-1-yl)acetate (1)

To a stirring solution of intermediate 35 (0.4 g, 1.0 mmol) in DMSO was added bis(pinacolato)diboron (0.77 g, 3 mmol) followed by dppf PdCl₂ (74 mg, 0.1 mmol), AcOK (0.3 g, 3 mmol) was added and the reaction mixture was refluxing under N₂ for 3 hours. After consumption of the starting material (by TLC), the mixture was diluted with water and extracted with EtOAc, Combined organic extracts were dried over anhydrous Na₂SO₄ and concentrated under reduced pressure to obtain crude product, which was purified by silica gel column chromatography to afford compound 1 (0.45 g, 71%) as yellow oil. LC-MS: m/z=443.12 [M+H]⁺

### tert-butyl 2-(5′-(2,4-dimethoxypyrimidin-5-yl)-2,5-dioxo-2′,3′-dihydrospiro[imidazolidine-4,1′-indene]-1-yl)acetate (3)

To a stirring solution of compd-1 (0.14 g, 3.2 mmol) in n-BuOH (5 ml) was added 5-iodo-2,4-dimethoxypyrimidine (0.1 g, 3.8 mmol), Na₂CO₃ (67 mg, 6.3 mmol), PPh₃ (13 mg, 0.05 mmol) and followed by Pd(OAc)₂ (8.3 mg, 0.03 mmol). The mixture was refluxing for 3 hours under N₂. After consumption of the starting material (by TLC), the solvent from reaction mixture was removed under reduced pressure to obtain crude product, which was purified by silica gel column chromatography to afford compound 3 (82 mg, 61%) as white solid. LC-MS: m/z=455.13 [M+H]⁺

### 2-(5′-(2,4-dioxohexahydropyrimidin-5-yl)-2,5-dioxo-2′,3′-dihydrospiro[imidazolidine-4,1′-indene]-1-yl)acetic acid (4)

To a stirring solution of compound 3 (80 mg, 0.18 mmol) in 2 mL MeOH was added cont. HCl (10 mL) and the mixture was refluxing for 2 days, After consumption of the starting material (by TLC), the solvent from reaction mixture was removed under reduced pressure to obtain crude product 4 (50 mg, 91%), which was used to the next step without any other purification. LC-MS: m/z=371.12 [M+H]⁺

To a stirring solution of intermediate-14 (28 mg, 0.16 mmol) in DMF (10 mL) was added compd-4 (50 mg, 0.14 mmol) and DIPEA (23 mg, 0.18 mmol) at room temperature and stirred for 2 mins, then HATU (62 mg, 0.16 mmol) was added and the reaction mixture was stirred at room temperature for 1 hour. The solvent from the reaction was concentrated under reduced pressure to obtain crude product, which was purified by silica gel column chromatography to afford 148-1 (10 mg, 22%) as an off-white solid. LC-MS: m/z=527.21 [M+H] at RT 1.42 (100% purity). ¹H NMR (400 MHz, MeOD) δ 7.57 (s, 1H), 7.51 (s, 1H), 7.47 (d, J=7.5 Hz, 1H), 7.44-7.36 (m, 3H), 7.30 (t, J=6.3 Hz, 2H), 7.20 (s, 1H), 4.84-4.69 (m, 2H), 4.60-4.39 (m, 1H), 4.35 (t, J=4.8 Hz, 1H), 3.84 (d, J=6.9 Hz, 1H), 3.37 (s, 1H), 3.24-3.09 (m, 2H), 2.84-2.68 (m, 1H), 2.36 (dd, J=13.5, 7.4 Hz, 1H), 1.41-1.18 (m, 3H), 0.96 (d, J=7.7 Hz, 1H), 0.57 (s, 1H), 0.33 (ddd, J=18.8, 11.5, 4.5 Hz, 3H).

### Example 149

A mixture of intermediate 27 (100 mg, 0.356 mmol), Pd(dppf)Cl₂ (26 mg, 0.0356 mmol) and CH₃ONa (42 mg, 0.712 mmol) in CH₃OH:H₂O (5:1, 6 mL) was stirred and at 100° C. under CO overnight. After consumption of the starting material (by TLC), the reaction mixture was concentrated under reduced pressure to obtain crude product. The crude product was Prep-HPLC to afford 149-1 (5 mg, 10%) as a white solid. LC-MS: m/z=462.2 [M+H]⁺. ¹H NMR (300 MHz, MeOD): δ 7.96 (d, J=13.2 Hz, 1H), 7.43 (dt, J=20.0, 7.7 Hz, 1H), 7.33-7.25 (m, 1H), 4.83-4.72 (m, 1H), 4.55-4.45 (m, 1H), 4.36 (t, J=4.0 Hz, 1H), 3.92-3.78 (m, 1H), 3.41-3.35 (m, 1H), 3.25-3.04 (m, 1H), 2.85-2.71 (m, 1H), 2.47-2.27 (m, 1H), 1.38-1.24 (m, 1H), 1.23 (dd, J=6.8, 4.3 Hz, 1H), 1.04-0.87 (m, 1H), 0.57 (dd, J=13.3, 9.6 Hz, 1H), 0.49-0.19 (m, 1H).

### Example 150

### tert-butyl 2-(2′,4′-dioxo-2,3-dihydrospiro[indene-1,5′-ozazolidine]-5-ylamino)acetate (1)

To a stirring solution of SM1 (280 mg, 1 mmol), K₃PO₄ (910 mg, 4.3 mmol), L-proline (130 mg, 1.1 mmol) and CuI (270 mg, 1.4 mmol) in DMSO (3 mL) was added tert-butyl 2-aminoacetate (160 mg, 1.2 mmol) at room temperature. The reaction mixture was stirred at 90° C. overnight then concentrated at 70° C. in vacuo. The residue was purified by silica gel column chromatography eluting with 50% EtOAc/Hexane to afford compound 1 (170 mg, 50%). TLC: 60% EtOAc/Hexane (R_{f}: 0.3)

### tert-butyl 2-(3′-(2-(benzyl((S)-1-cyclopropylethyl)amino)-2-oxoethyl)-2′,4′-dioxo-2,3-dihydrospiro[indene-1,5′-ozazolidine]-5-ylamino)acetate (2)

To a stirring solution of compound 1 (170 mg, 0.51 mmol) in DMF (3 mL) was added K₂CO₃ (150 mg, 1.02 mmol) at room temperature. To this added compound Intermediate-23 (150 mg, 0.51 mmol) at room temperature and the reaction mixture was stirred at room temperature for 0.5 h. The mixture was diluted with brine, extracted with EtOAc, the organic phase was washed with brine, dried over anhydrous Na₂SO₄ and concentrated and purified by silica gel column chromatography eluting with 30% EtOAC/Hexane to afford compound 2 (60 mg, 21%). TLC: 50% EtOAc/Hexane (R_{f}: 0.5)

A solution of compound 2 (60 mg, 0.1 mmol) in DCM (1 mL) was added 1 mL of TFA, the reaction mixture was stirred at room temperature for 1 hour then concentrated and purified with Pre-HPLC to afford compound 150-1 (15 mg, 30%) as white solid. TLC: 10% MeOH/DCM (Rf: 0.1). LC-MS: m/z=448 [M+H]⁺ (98% purity). ¹H NMR (400 MHz, CD₃OD-d4) δ 7.48-7.15 (m, 6H), 6.56 (d, J=12.0 Hz, 2H), 4.82-4.66 (m, 2H), 4.60-4.44 (m, 2H), 3.91 (s, 1H), 3.83 (s, 1H), 3.20-2.94 (m, 2H), 2.79-2.66 (m, 1H), 2.52 (dd, J=11.1, 6.2 Hz, 1H), 1.27 (dd, J=22.0, 6.7 Hz, 3H), 0.96 (s, 1H), 0.59 (d, J=5.0 Hz, 1H), 0.32 (d, J=17.7 Hz, 3H).

### Example 151

### (S)—N-(3′-(2-(benzyl(1-cyclopropylethyl)amino)-2-oxoethyl)-2′,4′-dioxo-2,3-dihydrospiro[indene-1,5′-ozazolidine]-5-yl)-3-oxobutanamide

A mixture of 4-methyleneoxetan-2-one SM1 (19 mg, 0.22 mmol) and (S)-2-(5-amino-2′,4′-dioxo-2,3-dihydrospiro[indene-1,5′-ozazolidine]-3′-yl)-N-benzyl-N-(1-cyclopropylethyl)acetamide Inter-18 (100 mg, 0.22 mmol) in toluene (2 mL) was degassed and sealed, which was stirred at 100° C. for 30 mins under microwave. The reaction mixture was concentrated under reduced pressure to afford (S)—N-(3′-(2-(benzyl(1-cyclopropylethyl)amino)-2-oxoethyl)-2′,4′-dioxo-2,3-dihydrospiro[indene-1,5′-ozazolidine]-5-yl)-3-oxobutanamide compound 1 (130 mg, crude) as an off-white solid. LC-MS: m/z=517.8 [M+H]⁺

### (E)-N-(3-cyanothiazolidin-2-ylidene)cyanamide (compound 2)

To a suspension of (E)-N-(thiazolidin-2-ylidene)cyanamide SM2 (127 mg, 1.0 mmol) in THF (5 mL) was added NaH (60% suspension, 48 mg, 1.2 mmol) under argon atmosphere at 0° C. The mixture was warmed to room temperature and stirred for 20 mins, a solution of BrCN (105 mg, 1.0 mmol) in THF(1 mL) was added dropwise, the mixture was stirred for 2 hours at room temperature. The mixture was quenched with NH₄Cl(sat, aq, 2 mL) and extracted with ethyl acetate(20 mL×2), combined the organic phase, which was dried over anhydrous sodium sulphate and concentrated to give crude product, which was purified by recrystallization (EtOH) to afford (E)-N-(3-cyanothiazolidin-2-ylidene)cyanamide compound 2 (48 mg, 32%). LC-MS: m/z=153.1 [M+H]⁺

### N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-cyano-3-oxobutanamide

To a suspension of (S)—N-(3′-(2-(benzyl(1-cyclopropylethyl)amino)-2-oxoethyl)-2′,4′-dioxo-2,3-dihydrospiro[indene-1,5′-ozazolidine]-5-yl)-3-oxobutanamide compound 1 (15 mg, 0.03 mmol) in THF (3 mL) was added NaH (60% suspension, 3 mg, 0.08 mmol) under argon atmosphere at 0° C. The mixture was warmed to room temperature and stirred for 20 mins, (E)-N-(3-cyanothiazolidin-2-ylidene)cyanamide compound 2 (9 mg, 0.06 mmol) was added and the mixture was stirred for 3 hours at room temperature. The mixture was quenched with NH₄Cl(sat, aq, 2 mL) and extracted with ethyl acetate(20 mL×2), combined the organic phase, which was dried over anhydrous sodium sulphate and concentrated to give crude product, which was purified by prep-HPLC to afford N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-cyano-3-oxobutanamide (10 mg, 64%). ¹H NMR (400 MHz, DMSO-d6): δ 11.40 (brs, 1H), 7.93-7.10 (m, 7H), 4.91-4.17 (m, 4H), 3.80-3.76 (m, 1H), 3.20-2.81 (m, 2H), 2.85-2.55 (m, 2H), 2.15 (s, 3H), 1.38-0.74 (m, 4H), 0.60-0.31 (m, 4H). LC-MS: m/z=543.1 [M+H]⁺ at room temperature 5.27 (95.55% purity)

To a suspension of (S)—N-(3′-(2-(benzyl(1-cyclopropylethyl)amino)-2-oxoethyl)-2′,4′-dioxo-2,3-dihydrospiro[indene-1,5′-ozazolidine]-5-yl)-3-oxobutanamide compound 1 (15 mg, 0.03 mmol) in THF (3 mL) was added NaH (60% suspension, 3 mg, 0.08 mmol) under argon atmosphere at 0° C. The mixture was warmed to room temperature and stirred for 20 mins, (E)-N-(3-cyanothiazolidin-2-ylidene)cyanamide compound 2 (9 mg, 0.06 mmol) was added and the mixture was stirred for 3 hours at room temperature. The mixture was quenched with NH₄Cl(sat, aq, 2 mL) and extracted with ethyl acetate(20 mL×2), combined the organic phase, which was dried over anhydrous sodium sulphate and concentrated to give crude product, which was purified by prep-HPLC to afford N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-cyano-3-oxobutanamide 151-1 (10 mg, 64%). LC-MS: m/z=543.1 [M+H]⁺ at RT 5.27 (95.55% purity). ¹H NMR (400 MHz, DMSO): δ 11.40 (brs, 1H), 7.93-7.10 (m, 7H), 4.91-4.17 (m, 4H), 3.80-3.76 (m, 1H), 3.20-2.81 (m, 2H), 2.85-2.55 (m, 2H), 2.15 (s, 3H), 1.38-0.74 (m, 4H), 0.60-0.31 (m, 4H).

### Example 152

### (S)—N-(1-(2-(benzyl(1-cyclopropylethyl)amino)-2-oxoethyl)-2,5-dioxo-2′,3′-dihydrospiro[imidazolidine-4,1′-indene]-5′-yl)-3-oxobutanamide

A mixture of 4-methyleneoxetan-2-one SM (7 mg, 0.075 mmol) and (S)-2-(5′-amino-2,5-dioxo-2′,3′-dihydrospiro[imidazolidine-4,1′-indene]-1-yl)-N-benzyl-N-(1-cyclopropylethyl)acetamide intermediate-37 (28 mg, 0.06 mmol) in toluene (2 mL) was degassed and sealed, which was stirred at 100° C. for 30 mins under microwave. The reaction mixture was concentrated under reduced pressure to obtain crude product, which was purified by Prep-HPLC to afford (S)—N-(1-(2-(benzyl (1-cyclopropylethyl)amino)-2-oxoethyl)-2,5-dioxo-2′,3′-dihydrospiro[imidazolidine-4,1′-indene]-5′-yl)-3-oxobutanamide 152-1 (20 mg, 60%) as an off-white solid. LC-MS: m/z=517.0 [M+H] at room temperature 4.05 (98.27% purity). ¹H NMR (400 MHz, CD₃OD-d4): δ 7.73-7.16 (m, 7H), 4.85-4.29 (m, 4H), 3.90-3.77 (m, 1H), 3.34-3.28 (m, 2H), 3.21-3.00 (m, 2H), 2.80-2.65 (m, 1H), 2.37-2.33 (m, 1H), 2.29 (s, 3H), 1.36-1.17 (m, 3H), 0.98-0.94 (m, 1H), 0.68-0.52 (m, 1H), 0.35-0.24 (m, 3H).

### Example 153

### 5-methylisoxazole-4-carbonyl chloride

To a solution of 5-methylisoxazole-4-carboxylic acid SM (500 mg, 4.0 mmol) in toluene (10 mL) was added SOCl₂ (720 mg, 6.0 mmol). The mixture was heated to reflux for 3 hours. The reaction mixture was concentrated under reduced pressure to obtain 5-methylisoxazole-4-carbonyl chloride compound 1 (550 mg, crude) without further purification.

### (S)—N-(1-(2-(benzyl(1-cyclopropylethyl)amino)-2-oxoethyl)-2,5-dioxo-2′,3′-dihydrospiro[imidazolidine-4,1′-indene]-5′-yl)-5-methylisoxazole-4-carboxamide (compound 2)

To a mixture of TEA (43 mg, 0.42 mmol), (S)-2-(5′-amino-2,5-dioxo-2′,3′-dihydrospiro[imidazolidine-4,1′-indene]-1-yl)-N-benzyl-N-(1-cyclopropylethyl)acetamide Inter-37 (60 mg, 0.14 mmol) and in dichloromethane (3 mL) was added a solution of 5-methylisoxazole-4-carbonyl chloride compound 1 (25 mg, 0.16 mmol) in dichloromethane (0.2 mL) at room temperature. The mixture was stirred at room temperature for 3 hours. The reaction mixture was poured to water (5 mL) and extracted with dichloromethane (5 mL×2) The organic phase was concentrated under reduced pressure to afford (S)—N-(1-(2-(benzyl(1-cyclopropylethyl)amino)-2-oxoethyl)-2,5-dioxo-2′,3′-dihydrospiro[imidazolidine-4,1′-indene]-5′-yl)-5-methylisoxazole-4-carboxamide compound 2 (98 mg, crude) without further purification. LC-MS: m/z=542.2 [M+H]⁺

### N-(1-(2-(benzyl((S)-1-cyclopropylethyl)amino)-2-oxoethyl)-2,5-dioxo-2′,3′-dihydrospiro[imidazolidine-4,1′-indene]-5′-yl)-2-cyano-3-oxobutanamide

To a solution of (S)—N-(1-(2-(benzyl(1-cyclopropylethyl)amino)-2-oxoethyl)-2,5-dioxo-2′,3′-dihydrospiro[imidazolidine-4,1′-indene]-5′-yl)-5-methylisoxazole-4-carboxamide compound 2 (98 mg, crude, 0.14 mmol) in methanol (2 mL) was added TEA (45 mg, 0.42 mmol), the mixture was stirred at room temperature for 30 mins. Water (1 mL) was added and the mixture was adjusted to pH=4 with 1N HCl. The reaction mixture was evaporated to dryness under reduced pressure to obtain crude product, which was purified by Prep-HPLC to afford N-(1-(2-(benzyl((S)-1-cyclopropylethyl)amino)-2-oxoethyl)-2,5-dioxo-2′,3′-dihydrospiro[imidazolidine-4,1′-indene]-5′-yl)-2-cyano-3-oxobutanamide (14 mg, 19% two steps) as off-white solid. ¹H NMR (400 MHz, DMSO-d6): δ 10.71 (brs, 1H), 8.76 (d, J=17.0 Hz, 1H), 7.71-7.10 (m, 7H), 4.84-4.56 (m, 2H), 4.51-4.09 (m, 2H), 3.73 (m, 1H), 3.18-2.86 (m, 2H), 2.22 (s, 3H), 1.33-1.02 (m, 3H), 0.96-0.92 (m, 1H), 0.61-0.04 (m, 4H). LC-MS: m/z=542.2 [M+H]⁺ at room temperature 4.76 (100% purity)

To a solution of (S)—N-(1-(2-(benzyl(1-cyclopropylethyl)amino)-2-oxoethyl)-2,5-dioxo-2′,3′-dihydrospiro[imidazolidine-4,1′-indene]-5′-yl)-5-methylisoxazole-4-carboxamide compound 2 (98 mg, crude, 0.14 mmol) in methanol (2 mL) was added TEA (45 mg, 0.42 mmol), the mixture was stirred at room temperature for 30 mins. Water (1 mL) was added and the mixture was adjusted to pH=4 with 1N HCl. The reaction mixture was evaporated to dryness under reduced pressure to obtain crude product, which was purified by Prep-HPLC to afford 153-1 (14 mg, 19% two steps) as off-white solid. LC-MS: m/z=542.2 [M+H]⁺ at RT 4.76 (100% purity). ¹H NMR (400 MHz, DMSO): δ 10.71 (brs, 1H), 8.76 (d, J=17.0 Hz, 1H), 7.71-7.10 (m, 7H), 4.84-4.56 (m, 2H), 4.51-4.09 (m, 2H), 3.73 (m, 1H), 3.18-2.86 (m, 2H), 2.22 (s, 3H), 1.33-1.02 (m, 3H), 0.96-0.92 (m, 1H), 0.61-0.04 (m, 4H).

### Example 154

### tert-butyl2-(3′-(2-(benzyl((S)-1-cyclopropylethyl)amino)-2-oxoethyl)-2′,4′-dioxo-2,3-dihydrospiro[indene-1,5′-ozazolidine]-5-yloxy)acetate (2)

To a stirring solution of intermediate 146-1 (50 mg, 0.115 mmol) in CH₃CN (5 mL) was added tert-butyl 2-bromoacetate compound 1 (33.4 mg, 0.1725 mmol) followed by K₂CO₃ (31.7 mg, 0.23 mmol) at room temperature and stirred at room temperature for 3 hours. The reaction mixture was diluted with water and extracted with EA. Combined organic extracts were dried over anhydrous Na₂SO₄ and concentrated under reduced pressure to obtain crude product, which was purified by silica gel column chromatography eluting with 50% EA/PE to afford compound 2 (40 mg, 63.5%). TLC: 50% EtOAc/Hexane (R_{f}: 0.4). LC-MS: m/z=549 [M+H]⁺

To a stirring solution of tert-butyl 2-(3′-(2-(benzyl((S)-1-cyclopropylethyl)amino)-2-oxoethyl)-2′,4′-dioxo-2,3-dihydrospiro[indene-1,5′-ozazolidine]-5-yloxy)acetate compound 2 (40 mg, 0.073 mmol) in DCM (2 mL) was added TFA (1 ml) at room temperature and stirred at room temperature for 1 hour. The reaction mixture was diluted with water and extracted with EA. Combined organic extracts were dried over anhydrous Na₂SO₄ and concentrated under reduced pressure to obtain crude product, which was purified by silica gel column chromatography eluting with 70% EA/PE to afford 154-1 (10 mg, 27.7%). LC-MS: m/z=493 [M+H]⁺ at RT 1.60 (100% purity). ¹H NMR (300 MHz, DMSO-d6) δ 7.45-7.11 (m, 6H), 6.90 (d, J=9.5 Hz, 2H), 4.57 (ddd, J=101.0, 15.7, 12.6 Hz, 8H), 3.84-3.30 (m, 1H), 3.21-2.92 (m, 2H), 2.73-2.57 (m, 1H), 1.29-1.07 (m, 4H), 1.06-0.82 (m, 1H), 0.24 (d, J=3.5 Hz, 4H).

### Example 155

### N-benzyl-N—((S)-1-cyclopropylethyl)-2-(5-(oxetan-3-ylamino)-2′,4′-dioxo-2,3-dihydrospiro[indene-1,5′-ozazolidine]-3′-yl)acetamide (3)

A mixture of SM1 (50 mg, 0.101 mmol), SM2 (15 mg, 0.202 mmol), K₃PO₄ (64.2 mg, 0.303 mmol), L-proline (13 mg, 0.111 mmol) and CuI (29 mg, 0.1515 mmol) in DMSO (2 ml) was stirred and at 100° C. and then microwave for 1 hour. After consumption of the starting material (by TLC), the reaction mixture was concentrated under reduced pressure to obtain crude product. The crude product was Prep-HPLC to afford 155-1 (10 mg, 20%) as a white solid. LC-MS: m/z=489.6 [M+H]⁺. ¹H NMR (400 MHz, MeOD) δ 7.43 (dt, J=15.3, 7.6 Hz, 1H), 7.37-7.20 (m, 1H), 6.48 (dd, J=15.4, 7.2 Hz, 1H), 4.99 (td, J=6.5, 2.8 Hz, 1H), 4.85-4.74 (m, 1H), 4.68-4.62 (m, 1H), 4.60-4.52 (m, 1H), 4.43 (t, J=6.4 Hz, 1H), 3.92-3.78 (m, for 1H), 3.41-3.34 (m, 1H), 3.15 (dt, J=14.9, 7.0 Hz, 1H), 3.07-2.93 (m, 1H), 2.79-2.66 (m, 1H), 2.62-2.45 (m, 1H), 1.36-1.27 (m, 1H), 1.25 (d, J=6.8 Hz, 1H), 0.97 (ddd, J=21.0, 13.5, 6.2 Hz, 1H), 0.69-0.51 (m, 1H), 0.46-0.21 (m, 1H).

### Example 156

### N-benzyl-N—((S)-1-cyclopropylethyl)-2-(2′,4′-dioxo-5-(tetrahydro-2H-pyran-4-ylamino)-2,3-dihydrospiro[indene-1,5′-ozazolidine]-3′-yl)acetamide (2)

A mixture of SM1 (50 mg, 0.101 mmol), SM2 (10 mg, 0.202 mmol), K₃PO₄ (64.2 mg, 0.303 mmol), L-proline (13 mg, 0.111 mmol) and CuI (29 mg, 0.1515 mmol) in DMSO (2 mL) was stirred and at 100° C. and microwave for 1 hour. After consumption of the starting material (by TLC), the reaction mixture was concentrated under reduced pressure to obtain crude product. The crude product was Prep-HPLC to afford 156-1 (17 mg, 25%) as a white solid. LC-MS: m/z=518.6 [M+H]⁺. ¹H NMR (400 MHz, MeOD) δ 7.53-7.36 (m, 1H), 7.36-7.26 (m, 1H), 7.26-7.13 (m, 1H), 6.66-6.51 (m, 1H), 4.86-4.76 (m, 1H), 4.73-4.48 (m, 1H), 4.44 (t, J=6.3 Hz, 1H), 3.99 (d, J=11.0 Hz, 1H), 3.90-3.81 (m, 1H), 3.56 (dd, J=12.8, 10.5 Hz, 1H), 3.21-3.08 (m, 1H), 3.08-2.91 (m, 1H), 2.85-2.63 (m, 1H), 2.60-2.40 (m, 1H), 2.01 (d, J=12.9 Hz, 1H), 1.59-1.42 (m, 1H), 1.32 (d, J=8.2 Hz, 1H), 1.25 (d, J=6.8 Hz, 1H), 0.94 (dd, J=19.6, 12.6 Hz, 1H), 0.70-0.53 (m, 1H), 0.35 (ddd, J=18.8, 11.4, 5.2 Hz, 1H).

### Example 157

### N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-hydroxypropanamide (3)

A mixture of compound intermediate 18 (50 mg, 0.117 mmol), 2-hydroxypropanoic acid (15.7 mg, 0.175 mmol), HOBT (23.6 mg, 0.175 mmol) and EDCI (33.4 mg, 0.175 mmol) in DCM (5 mL) was stirred at room temperature overnight. After consumption of the starting material (by TLC), the reaction mixture was concentrated under reduced pressure to obtain crude product. The crude product was Prep-HPLC to afford 157-1 (14 mg, 35%) as a white solid. LC-MS: m/z=506.2 [M+H]⁺. ¹H NMR (400 MHz, MeOD-d6): δ 7.76 (d, J=15.7 Hz, 1H), 7.54-7.37 (m, 1H), 7.34-7.24 (m, 1H), 4.79 (dd, J=10.4, 7.2 Hz, 1H), 4.70-4.50 (m, 1H), 4.45 (t, J=6.9 Hz, 1H), 4.34-4.20 (m, 1H), 3.93-3.80 (m, 1H), 3.41-3.35 (m, 1H), 3.17 (ddd, J=16.0, 13.2, 7.5 Hz, 1H), 2.88-2.73 (m, 1H), 2.65-2.46 (m, 1H), 1.56 (d, J=6.7 Hz, 1H), 1.41 (dd, J=29.3, 10.0 Hz, 1H), 1.32 (d, J=6.5 Hz, 1H), 1.24 (d, J=6.8 Hz, 1H), 0.98 (d, J=5.2 Hz, 1H), 0.67-0.46 (m, 1H), 0.42-0.24 (m, 1H).

### Example 158

### (E)-methyl 4-(piperidin-1-yl)but-2-enoate (3)

To a solution of (E)-methyl 4-bromobut-2-enoate 1 (1.7 g, 10 mmol) in DCM (20 mL) was added piperidine (1.8 g, 20 mmol) at room temperature under N₂, the reaction mixture was stirred at room temperature for 2 hours. Concentrated under reduced pressure to obtain crude compound, which was purified by silica gel column chromatography (EtOAc/Hexane 1:3) to afford compound 3 (1.5 g, 81%) as yellow oil. LC-MS: m/z=184.1 [M+H]⁺ (95% purity)

### (E)-4-(piperidin-1-yl)but-2-enoic acid (4)

To a solution of (E)-methyl 4-(piperidin-1-yl)but-2-enoate 3 (1.5 g, 8.2 mmol) in dioxane (20 mL) was added HCl (8.2 mL, 2N in H₂O), the reaction mixture was stirred at 80° C. for 16 h. After consumption of the starting material (by TLC), concentrated under reduced pressure to obtain crude product, which was washed with EtOAc to afford compound 4 (1.45 g, 86%) as brown solid. LC-MS: m/z=168.1 [M−H]⁺ (95% purity)

### (E)-4-(piperidin-1-yl)but-2-enoyl chloride (5)

To a solution of (E)-4-(piperidin-1-yl)but-2-enoic acid 2 (103 mg, 0.5 mmol) in dry DCM (20 mL) was added oxalyl dichloride (256 mg, 2 mmol) and DMF (4 mg, 0.05 mmol) at 0° C. under N₂, then stirred at room temperature for 1 hour, concentrated under reduced pressure to obtain crude compound 5 (130 mg, 116%).

To a solution of compound 5 (130 mg, 0.5 mmol) intermediate 37 (65 mg, 0.15 mmol) in THF (10 ml) was added TEA (90 mg, 0.9 mmol) at room temperature under N2 and stirred at room temperature for 2 hours. After consumption of the starting material (by TLC) diluted with water and extracted with EtOAc. Combined organic extracts were dried over anhydrous MgSO₄ and concentrated under reduced pressure to obtain crude product, which was purified by Prep-HPLC (MeCN/H₂O 2:1) to afford 158-1 (27 mg, 31%) as white solid. LC-MS: m/z=584.3 [M+H]⁺ at RT 3.75 (92.95% purity). ¹H NMR (300 MHz, CD₃OD) δ 7.71 (s, 1H), 7.51-7.05 (m, 7H), 6.93-6.72 (m, 1H), 6.51 (d, J=15.0 Hz, 1H), 4.82-4.63 (m, 2H), 4.57-4.24 (m, 2H), 3.99-3.75 (m, 2H), 3.52 (d, J=12.1 Hz, 2H), 3.30 (dd, J=6.9, 5.4 Hz, 2H), 3.12 (s, 2H), 2.95 (t, J=11.9 Hz, 2H), 2.72 (dd, J=13.4, 5.6 Hz, 1H), 2.33 (dd, J=13.1, 6.8 Hz, 1H), 2.02-1.66 (m, 4H), 1.50 (d, J=12.3 Hz, 1H), 1.38-1.10 (m, 3H), 1.03-0.87 (m, 1H), 0.68-0.17 (m, 4H).

### Example 159

### oxetane-3-carboxylic acid(1)

To a solution of SM1 (500 mg, 5.68 mmol) in MeCN(5 mL) and H₂O (2 mL) was added NaIO₄ (3.65 g, 17.1 mmol) and RuCl₃ (117.6 mg, 0.56 mmol) at room temperature. The resulting mixture was stirred at room temperature overnight. Quenched with saturated aqueous solution of NH₄Cl and extracted with DCM(20 mL×3). The organic layer was combined and concentrated to give crude compound 1 (300 mg, 80% purity) as oil. TLC: 10% MeOH/DCM (Rf: 0.5) 1-(oxetan-3-yl)ethanone(2):

To a solution of compound 1 (300 mg, 80% purity) in DCM(5 mL) was added MeNHOMe.HCl (229 mg, 2.35 mmol), NEt₃ (594 mg, 5.88 mmol) and T₃P(1.25 g, 3.92 mmol) at 0° C. The resulting solution was stirred at room temperature for 2 hours. Evaporate the solvent to give crude Weinreb amide. Diluted it in THF(5 mL), MeMgBr in ether(0.83 mL, 2.48 mmol) was added at 0° C. Stirred for 2 hours and warmed it up to room temperature slowly. Quenched with saturated aqueous solution of NH₄Cl and extracted with DCM (20 mL×3). The organic layer was combined and concentrated to give crude compound 2 (200 mg, 80% purity) as oil. TLC: 10% MeOH/DCM (Rf: 0.8)

### N-benzyl-1-(oxetan-3-yl)ethanamine(3)

To a solution of compound 2 (200 mg, 80% purity) in MeOH(5 mL) was added BnNH₂ (257.1 mg, 2.4 mmol) and NaBH(OAc)₃ (847.4 mg, 4.0 mmol). The resulting solution was stirred at room temperature for 2 hours. After filtration, purified by prep-HPLC(MeCN in H₂O (0.1% TFA) from 10%-50%) to give compound 3 (100 mg, 0.52 mmol).

To a solution of compound 3 (100 mg, 0.52 mmol) in DCM (4 mL) was added intermediate 9, T3P (330.9 mg, 1.04 mmol) and NEt₃ (157.6 mg, 1.56 mmol). The resulting solution was stirred at room temperature for 3 hours. Quenched by water and extracted by DCM(10 mL×3). The organic layer was combined, concentrated and purified by chromatography(MeOH in DCM 0-5%) to give 159-1. (10 mg, 0.023 mmol, 4.4% yield). LC-MS: m/z=432.1 [M−H]⁺ at RT 4.40 (80.45% purity). ¹H NMR (400 MHz, CDCl₃) δ 7.49-7.37 (m, 4H), 7.32 (m, 9H), 7.16 (t, J=6.7 Hz, 1H), 5.99-5.76 (m, 2H), 5.10-4.87 (m, 2H), 4.84-4.54 (m, 3H), 4.53-4.42 (m, 3H), 4.39-4.21 (m, 7H), 4.17 (d, J=5.8 Hz, 1H), 4.08 (m, 1H), 3.89 (d, J=5.7 Hz, 1H), 3.82 (d, J=4.5 Hz, 1H), 3.33-3.17 (m, 2H), 3.18-2.97 (m, 4H), 2.94-2.76 (m, 2H), 2.32 (m, 2H), 1.30 (d, J=18.3 Hz, 1H), 1.23 (dd, J=6.4, 2.6 Hz, 1H), 1.11 (dd, J=6.8, 2.7 Hz, 3H).

### Example 160

To a solution of intermediate-28 (100 mg, 0.2 mmol) and azetidine-3-carboxamide (40 mg, 0.4 mmol) in DMSO (6 mL) was added K3PO4 (183 mg, 0.86 mmol), CuI (54 mg, 0.28 mmol) and L-proline (33 mg, 0.22 mmol) under N₂. Then stirred at 90° C. for 16 h. After consumption of the starting material (by TLC), diluted with water and extracted with EtOAc. Combined organic extracts were dried over anhydrous MgSO₄ and concentrated under reduced pressure to obtain crude product, which was purified by Prep-HPLC (MeCN/H₂O 3:2) to afford 160-1 (25 mg, 24%) as white solid. LC-MS: m/z=517.2 [M+H]⁺ at RT 4.41 (98.74% purity). ¹H NMR (400 MHz, DMSO-d6)¹H NMR (301 MHz, DMSO) δ 7.50 (s, 1H), 7.44-7.15 (m, 6H), 7.02 (s, 1H), 6.45-6.29 (m, 2H), 4.79 (s, 1H), 4.66 (s, 1H), 4.34 (m, 2H), 3.96 (m, 3H), 3.42 (m, 2H), 3.06 (m, 3H), 2.59 (m, 1.5H), 2.45 (s, 0.5H), 1.30-1.09 (m, 4H), 0.97 (s, 1H), 0.60-0.09 (m, 4H).

### Example 161

### N-Benzyl-1,1-dicyclopropylmethanamine (1)

To a stirring solution of SM1 (4.87 g, 45.5 mmol) in 1,2-dichloroethane (50 mL) was added dicyclopropylmethanone (5 g, 45.5 mmol), tetraisopropoxytitanium (15.5 g, 54.6 mmol). The mixture was stirred at room temperature for 2 hours, then NaBH₃CN (5.71 g, 91 mmol) was added at 0° C. and the resulting mixture was stirred at room temperature overnight. The reaction mixture was diluted with water and extracted with EtOAc, washed by brine. Combined organic extracts were dried over anhydrous Na₂SO₄ and concentrated under reduced pressure to obtain crude product, which was purified by silica gel column chromatography eluting with 33% EtOAc/PE to afford compound 1 (5.95 g, 65%) as a yellow solid. LC-MS: m/z=202 [M+H]⁺

### N-Benzyl-2-bromo-N-(dicyclopropylmethyl)acetamide (2)

To a stirring solution of compound 1 (845 mg, 4.2 mmol) in DCM (5 mL) was added 2-bromoacetyl bromide (847.8 mg, 4.2 mmol) followed by Et₃N (424 mg, 4.2 mmol) at 0° C. and the reaction mixture was stirred at room temperature for 2 hours, then concentrated under reduced pressure to obtain crude product, which was purified by silica gel column chromatography eluting with 33% EtOAc/PE to afford compound 2 (606 mg, 45%) as a clear oil. LC-MS: m/z=322/324 [M+H]⁺

### 2-(5-Acetamido-2′,4′-dioxo-2,3-dihydrospiro[indene-1,5′-ozazolidine]-3′-yl)-N-benzyl-N-(dicyclopropylmethyl)acetamide (3)

To a stirring solution of compound 2 (248 mg, 0.77 mmol) in DMF (2 mL) was added Intermediate 8 (200 mg, 0.77 mmol), followed by K₂CO₃ (212.5 mg, 1.54 mmol). The reaction mixture was stirred at room temperature overnight and diluted with water and extracted with EtOAc, washed by brine. Combined organic extracts were dried over anhydrous Na₂SO₄ and concentrated under reduced pressure to obtain crude product, which was purified by silica gel column chromatography eluting with 5% MeOH/DCM to afford compound 3 (366 mg, 95%) as a grey solid. LC-MS: m/z=502 [M+H]⁺

### 2-(5-amino-2′,4′-dioxo-2,3-dihydrospiro[indene-1,5′-ozazolidine]-3′-yl)-N-benzyl-N-(dicyclopropylmethyl)acetamide (4)

To a stirring solution of compound 3 (366 mg, 0.73 mmol) in MeOH (4 mL), H₂O (4 mL) was added HCl (1.5 mL). The reaction mixture was stirred at 60° C. for 2 hours and cooled to room temperature, neutralized by 4N NaOH until pH=8, extracted with EtOAc, washed by brine. Combined organic extracts were dried over anhydrous Na₂SO₄ and concentrated under reduced pressure to obtain crude product, which was purified by prep-TLC eluting with 5% MeOH/DCM to afford compound 4 (50 mg, 15%) as a yellow solid. LC-MS: m/z=460 [M+H]⁺

To a stirring solution of compound 4 (25 mg, 0.0545 mmol) in DCM (0.5 mL) was added 2-amino-2-methylpropanoyl chloride hydrochloride (17.2 mg, 0.109 mmol), followed by Py (0.5 mL) at room temperature and stirred at room temperature for 1 hour. After consumption of the starting material (by TLC), the reaction mixture was concentrated under reduced pressure to obtain crude product, which was purified by silica gel column chromatography eluting with 10% MeOH/DCM afford 161-1 (20 mg, 68%) as a white solid. LC-MS: m/z=545.2 [M+H]⁺. ¹H NMR (400 MHz, MeOD): δ 7.75 (s, 1H), 7.55-7.18 (m, 7H), 4.93 (s, 1H), 4.87 (s, 1H), 4.58-4.39 (m, 2H), 3.36 (dd, J=15.7, 5.5 Hz, 1H), 3.28-3.06 (m, 2H), 2.86-2.74 (m, 1H), 2.65-2.50 (m, 1H), 1.51 (s, 6H), 1.14-0.85 (m, 3H), 0.64-0.43 (m, 2H), 0.47-0.28 (m, 5H).

### Example 162

### N-Benzyl-1,1-dicyclopropylethanamine (1)

To a stirring solution of SM1 (4.87 g, 45.5 mmol) in 1,2-dichloroethane (50 mL) was added dicyclopropylmethanone (5 g, 45.5 mmol), tetraisopropoxytitanium (15.5 g, 54.6 mmol). The mixture was stirred at room temperature for 2 hours, then NaBH₃CN (5.71 g, 91 mmol) was added at 0° C. and the resulting mixture was stirred at room temperature overnight. The reaction mixture was diluted with water and extracted with EtOAc, washed by brine. Combined organic extracts were dried over anhydrous Na₂SO₄ and concentrated under reduced pressure to obtain crude product, which was purified by silica gel column chromatography eluting with 33% EtOAc/PE to afford compound 1 (5.95 g, 65%) as a yellow solid. LC-MS: m/z=202 [M+H]⁺

### N-Benzyl-2-bromo-N-(dicyclopropylmethyl)acetamide (2)

To a stirring solution of compound 1 (845 mg, 4.2 mmol) in DCM (5 mL) was added 2-bromoacetyl bromide (847.8 mg, 4.2 mmol) followed by Et₃N (424 mg, 4.2 mmol) at 0° C. and the reaction mixture was stirred at room temperature for 2 hours, then concentrated under reduced pressure to obtain crude product, which was purified by silica gel column chromatography eluting with 33% EtOAc/PE to afford compound 2 (606 mg, 45%) as a clear oil. LC-MS: m/z=322/324 [M+H]⁺

### 2-(5-Acetamido-2′,4′-dioxo-2,3-dihydrospiro[indene-1,5′-ozazolidine]-3′-yl)-N-benzyl-N-(dicyclopropylmethyl)acetamide (3)

To a stirring solution of compound 2 (248 mg, 0.77 mmol) in DMF (2 mL) was added Intermediate 8 (200 mg, 0.77 mmol), followed by K₂CO₃ (212.5 mg, 1.54 mmol). The reaction mixture was stirred at room temperature overnight and diluted with water and extracted with EtOAc, washed by brine. Combined organic extracts were dried over anhydrous Na₂SO₄ and concentrated under reduced pressure to obtain crude product, which was purified by silica gel column chromatography eluting with 5% MeOH/DCM to afford compound 3 (366 mg, 95%) as a grey solid. LC-MS: m/z=502 [M+H]⁺

### 2-(5-amino-2′,4′-dioxo-2,3-dihydrospiro[indene-1,5′-ozazolidine]-3′-yl)-N-benzyl-N-(dicyclopropylmethyl)acetamide (4)

To a stirring solution of compound 3 (366 mg, 0.73 mmol) in MeOH (4 mL), H₂O (4 mL) was added HCl (1.5 mL). The reaction mixture was stirred at 60° C. for 2 hours and cooled to room temperature, neutralized by 4N NaOH until pH=8, extracted with EtOAc, washed by brine. Combined organic extracts were dried over anhydrous Na₂SO₄ and concentrated under reduced pressure to obtain crude product, which was purified by prep-TLC eluting with 5% MeOH/DCM to afford compound 4 (50 mg, 15%) as a yellow solid. LC-MS: m/z 460 [M+H]⁺

To a stirring solution of compound 4 (25 mg, 0.0545 mmol) in DCM (1 mL) was added triphosgene (6.5 mg, 0.0218 mmol) followed by Et₃N (6.6 mg, 0.0654 mmol) at 0° C. and the reaction mixture was stirred at room temperature for 3.5 hours. Then 2 M methanamine in THF (0.27 mL, 0.545 mmol) was added at room temperature and the resulting mixture was stirred at room temperature for 2 hours and concentrated under reduced pressure to obtain crude product, which was purified by Prep-HPLC (50%-70% MeCN/H₂O, 0.1% FA) to afford 162-1 (15 mg, 54%) as a white solid. LC-MS: m/z=517.2 [M+H]⁺. ¹H NMR (400 MHz, MeOD): δ 7.59-7.16 (m, 8H), 4.93 (s, 2H), 4.48 (dd, J=33.1, 4.9 Hz, 2H), 3.22 (dd, J=15.5, 7.6 Hz, 1H), 3.10 (s, 1H), 2.79 (s, 3H), 2.77-2.70 (m, 1H), 2.56 (s, 1H), 1.32 (s, 1H), 1.04 (s, 2H), 0.71-0.25 (m, 8H).

### Example 163

### N-benzyl-N—((S)-1-cyclopropylethyl)-2-(5-hydroxy-2′,4′-dioxo-2,3-dihydrospiro[indene-1,5′-ozazolidine]-3′-yl)acetamide (2)

To a solution of compound 1 (1.4 g, 2.57 mmol) in MeOH (10 mL) was added dropwise 30% H₂O₂ (0.6 g, 5.15 mmol). The mixture was stirred at room temperature for 1.5 hours. The mixture was quenched with NaHSO₃ (a.q) and extracted with EtOAc (10 mL×3), and the combined organic layers were washed with brine, dried over Na₂SO₄, filtered, and concentrated under reduced pressure. The crude was purified by flash chromatography on silica gel using a mixture of (PE/EtOAc 50/50) as eluent to afford compound 2 (0.8 g, 69%) as an oil. LC-MS: m/z 435 [M+H]⁺

To a mixture of compound 2 (55 mg, 0.127 mmol), SM1 (17 mg, 0.19 mmol) and PPh₃ (50 mg, 0.19 mmol) in THF (3 mL) was added DIAD (38 mg, 0.19 mmol) under nitrogen at 0° C. The mixture was allowed to warm to room temperature and stirred overnight. The solvent was removed under reduced pressure. The residue was purified by flash chromatography eluted with DCM/MeOH (100:1 to 10:1) to afford compound 163-1 (20 mg, 31%) as an off-white solid. LC-MS: m/z=506 [M+H]⁺ at RT 1.39 (95.19% purity). ¹H NMR (400 MHz, MeOD): δ 7.49-7.38 (m, 3H), 7.35-7.19 (m, 3H), 7.03-6.89 (m, 2H), 4.85-4.75 (m, 2H), 4.70-4.50 (m, 1H), 4.45 (t, J=6.9 Hz, 1H), 4.28-4.19 (m, 2H), 3.85 (s, 1H), 3.36 (s, 1H), 3.29-3.18 (m, 1H), 3.17-3.02 (m, 3H), 2.89-2.72 (m, 1H), 2.66-2.51 (m, 7H), 1.29 (dd, J=29.5, 6.8 Hz, 4H), 0.99 (d, J=4.8 Hz, 1H), 0.68-0.53 (m, 1H), 0.45-0.20 (m, 3H).

### Example 164

To a stirring solution of compound 143-1 (47 mg, 0.1 mmol) and NaN₃ (7 mg, 0.11 mmol) in DMSO/H₂O (v/v, 9:1, 2 mL) was added 1 M CuSO₄ aq (0.4 mL, 0.4 mmol) followed by 1 M Vc-Na aq (0.6 mL, 0.6 mmol) at room temperature. The reaction mixture was heated to 70° C. for 1.5 hour. The reaction mixture was purified by Prep-HPLC to afford compound 164-1 (10 mg, 19%) as a white solid. LC-MS: m/z=516.2 [M+H]⁺ at RT 4.65 (99.88% purity). ¹H NMR (300 MHz, CD₃OD) δ 7.90 (s, 1H), 7.48-7.14 (m, 6H), 6.96 (dd, J=21.4, 13.4 Hz, 2H), 5.23 (s, 2H), 4.77 (d, J=8.1 Hz, 2H), 4.62-4.38 (m, 2H), 3.88-3.78 (m, 0.5H), 3.41-3.32 (m, 0.5H), 3.22-3.01 (m, 2H), 2.83-2.70 (m, 1H), 2.61-2.49 (m, 1H), 1.26 (dd, J=22.6, 6.5 Hz, 3H), 1.04-0.89 (m, 1H), 0.60-0.24 (m, 4H).

### Example 165

### N-benzyl-N—((S)-1-cyclopropylethyl)-2-(2′,4′-dioxo-5-(6-oxo-1,6-dihydropyridin-3-yl)-2,3-dihydrospiro[indene-1,5′-ozazolidine]-3′-yl)acetamide

A solution of 5-(4,4,5,5-tetramethyl-1,3,2-dioxaborolan-2-yl)pyridin-2-ol SM1 (0.05 g, 0.23 mmol), N-benzyl-2-(5-bromo-2′,4′-dioxo-2,3-dihydrospiro[indene-1,5′-ozazolidine]-3′-yl)-N—((S)-1-cyclopropylethyl)acetamide SM2 (0.112 g, 0.23 mmol), Pd(dppf)₂Cl₂ (0.085 g, 0.0115 mmol) and K₂CO₃ (0.095 g, 0.69 mmol) dissolved with dioxane and H₂O (10 mL/1 mL) in sealed tube was stirred at 90° C. overnight. After consumption of the starting material (by LC-MS), the solvent from reaction mixture was removed under reduced pressure, the residue was diluted with brine and extracted with EtOAc. Combined organic extracts were dried over anhydrous Na₂SO₄ and concentrated under reduced pressure to obtain crude product, which was purified by Prep-HPLC [H₂O-ACN(0.05% TFA)] to afford compound 165-1 (0.046 g, 39%) as a white liquid. ¹H NMR (400 MHz, DMSO-d₆): δ 11.96 (s, 1H), 7.89-7.18 (m, 10H), 6.45 (dd, J=9.5, 4.2 Hz, 1H), 4.82-4.28 (m, 5H), 3.25-3.05 (m, 3H), 2.75-2.66 (m, 1H), 1.18 (dd, J=27.0, 6.4 Hz, 3H). 0.98 (s, 1H), 0.56-0.13 (m, 4H). LC-MS: m/z=512.2 [M+H]⁺ (9′9.36% purity, 214 nm)

### Example 166

To a stirring solution of compound 1 (80 mg, 0.184 mmol) in DMF (3 mL) was added Cs₂CO₃ (180 mg, 0.552 mmol), the mixture was stirred at room temperature for 30 min. Then SM1 (60 mg, 0.552 mmol) was added, and the mixture was stirred at room temperature overnight. The reaction mixture was diluted with water and extracted with DCM. Combined organic extracts were dried over anhydrous Na₂SO₄ and concentrated under reduced pressure. The residue was purified by Prep-HPLC eluted with H₂O/MeCN (1:1) to afford compound 166-1 (4 mg, 4.5%) as an off-white solid. LC-MS: m/z=479 [M+H]⁺ at RT 1.59 (88.58% purity). ¹H NMR (400 MHz, MeOD): δ 7.54-7.16 (m, 6H), 6.92 (dd, J=19.7, 11.5 Hz, 2H), 4.79 (dd, J=11.6, 8.5 Hz, 2H), 4.60 (dd, J=24.1, 14.1 Hz, 1H), 4.45 (t, J=6.7 Hz, 1H), 4.08 (dd, J=8.5, 3.9 Hz, 2H), 3.88 (ddd, J=14.1, 9.4, 4.6 Hz, 2H), 3.37 (d, J=8.8 Hz, 1H), 3.30-3.03 (m, 2H), 2.87-2.72 (m, 1H), 2.65-2.50 (m, 1H), 1.29 (dd, J=29.4, 6.8 Hz, 4H), 0.98 (d, J=5.4 Hz, 1H), 0.67-0.53 (m, 1H), 0.47-0.21 (m, 3H).

### Example 167

### 5-(4,4,5,5-tetramethyl-1,3,2-dioxaborolan-2-yl)-2,3-dihydrospiro[indene-1,5′-ozazolidine]-2′,4′-dione (1)

A mixture of 5-bromo-2,3-dihydrospiro[indene-1,5′-ozazolidine]-2′,4′-dione Intermediate-25 (0.5 g, 1.77 mol), 4,4,4′,4′,5,5,5′,5′-octamethyl-2,2′-bi(1,3,2-dioxaborolane) (1.35 g, 5.32 mmol), Pd(dppf)Cl₂ (130 mg, 0.177 mmol) and AcOK (0.5 g, 5.32 mmol) in DMSO (8 mL) was heated at 100° C. under nitrogen. After cooled, the mixture was poured into water and extracted with EtOAc (10 mL×3) and the combined organic layers were washed with brine, dried over Na₂SO₄, filtered, and concentrated under reduced pressure. The crude was purified by flash chromatography on silica gel using a mixture of (PE/EtOAc 50/50) as eluent to afford compound 1 (0.5 g, 83%) as an oil. LC-MS: m/z=328 [M+H]⁺

### N-benzyl-N—((S)-1-cyclopropylethyl)-2-(2′,4′-dioxo-5-(4,4,5,5-tetramethyl-1,3,2-dioxaborolan-2-yl)-2,3-dihydrospiro[indene-1,5′-ozazolidine]-3′-yl)acetamide (2)

To a stirring solution of compound 1 (0.2 g, 0.61 mmol) in MeCN (5 mL) was added Intermediate-23 (0.18 g, 0.61 mmol) followed by K₂CO₃ (0.12 g, 0.79 mmol) at room temperature. The reaction mixture was stirred for at room temperature overnight. The reaction mixture was quenched with NH₄Cl (a.q) and extracted with EtOAc. Combined organic extracts were dried over anhydrous Na₂SO₄ and concentrated under reduced pressure. The residue was purified by silica gel column chromatography eluted with PE/EtOAc (4:1) to afford compound 2 (0.16 g, 50%) as an oil. LC-MS: m/z=545 [M+H]⁺

To a stirring solution of compound 2 (70 mg, 0.129 mmol) in THF/H₂O (4 mL/1 mL) was added NaIO₄ (83 mg, 0.386 mmol). The resulting suspension was stirred at room temperature for 30 min, and then treated with 3 M HCl (0.09 mL, 0.258 mmol). The mixture was stirred for 2.5 hours. The supernatant was decanted away from the solids, rining forward with THF. Combined organic extracts were washed with brine and dried over anhydrous Na₂SO₄ and concentrated under reduced pressure. The residue was purified by Prep-HPLC eluted with H₂O/MeCN (75:25 to 45:55) to afford compound 167-1 (20 mg, 20%) as an off-white solid. LC-MS: m/z=463 [M+H]⁺ at RT 1.56 (100% purity). ¹H NMR (400 MHz, MeOD): δ 7.81-7.36 (m, 5H), 7.36-7.11 (m, 3H), 4.80 (dd, J=12.3, 8.5 Hz, 2H), 4.74-4.51 (m, 1H), 4.44 (dd, J=19.1, 11.4 Hz, 1H), 3.94-3.80 (m, 1H), 3.37 (d, J=2.3 Hz, 1H), 3.30-3.10 (m, 2H), 2.88-2.71 (m, 1H), 2.58 (dd, J=9.0, 5.0 Hz, 1H), 1.29 (dd, J=28.7, 6.7 Hz, 3H), 1.05-0.90 (m, 1H), 0.68-0.52 (m, 1H), 0.39-0.27 (m, 3H).

### Example 168

To a solution of intermediate 27 (50 mg, 0.1 mmol) and azetidin-3-ylmethanamine (18 mg, 0.2 mmol) in DMSO (3 mL) was added K₃PO₄ (127 mg, 0.6 mmol), CuI (21 mg, 0.11 mmol) and L-proline (13 mg, 0.11 mmol) under N₂. Then stirred at 90° C. for 16 hours. After consumption of the starting material (by TLC), diluted with water and extracted with EtOAc. Combined organic extracts were dried over anhydrous MgSO₄ and concentrated under reduced pressure to obtain crude product, which was purified by Prep-HPLC (MeCN/H₂O 3:2) to afford 168-1 (10 mg, 20%) as yellow solid. LC-MS: m/z=504.2 [M+H]⁺ at RT 4.58 (93.90% purity). ¹H NMR (400 MHz, DMSO-d6) δ 7.46-7.15 (m, 6H), 6.33 (m, 2H), 4.78 (s, 2H), 4.66 (m, 1H), 4.33 (m, 2H), 3.83 (m, 3H), 3.57 (s, 4H), 3.06 (m, 1H), 2.96 (m, 1H), 2.79 (s, 1H), 2.61 (m, 1H), 2.47-2.35 (m, 1H), 1.17 (dd, J=27.6, 5.4 Hz, 3H), 1.04-0.87 (m, 1H), 0.57-0.09 (m, 4H).

### Example 169

To a solution of intermediate 33 (50 mg, 0.11 mmol) in pyridine (1 mL) and DCM (1 mL) was added intermediate 36 (36 mg, 0.23 mmol) under N₂. Then the mixture was stirred at room temperature for 1 hour. After consumption of the starting material (by TLC), the reaction mixture concentrated under reduced pressure to obtain crude product, which was purified by combiflash (DCM/MeOH 10:1) to afford 169-1 (44 mg, 80%) as white solid. LC-MS: m/z=518.1 [M+H]⁺ at RT 3.55 (97.09% purity). ¹H NMR (300 MHz, DMSO-d6) δ 8.80 (d, J=13.4 Hz, 2H), 7.68 (s, 1H), 7.53 (d, J=7.1 Hz, 1H), 7.42-7.34 (m, 2H), 7.32-7.14 (m, 4H), 4.68 (d, J=37.8 Hz, 2H), 4.43-4.15 (m, 2H), 3.80-3.65 (m, 1H), 3.02 (d, J=4.5 Hz, 2H), 2.57 (d, J=6.7 Hz, 1H), 2.22 (dd, J=13.1, 8.1 Hz, 1H), 1.54 (s, 5H), 1.45-1.32 (m, 2H), 1.15 (dd, J=24.9, 6.5 Hz, 3H), 0.95 (s, 1H), 0.52-0.13 (m, 4H).

### Example 170

### 2-(5-bromo-2′,4′-dioxo-2,3-dihydrospiro[indene-1,5′-ozazolidine]-3′-yl)-N—((S)-1

To a stirring solution of (S)-2-bromo-N-(1-cyclopropylethyl)-N-(4-fluorobenzyl)acetamide SM (200 mg, 0.64 mmol) and Intermediate-25 (180 mg, 0.64 mmol) in DMF (4 mL) was added K₂CO₃ (176 mg, 1.27 mmol) and stirred at room temperature for 1 hour. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 50% EtOAc/PE to afford compound 1 (220 mg, 67%) as a yellow solid. LC-MS: m/z 5=515.2/517.2 [M+H]⁺

To a stirring solution of compound 1 (118 mg, 0.23 mmol) and compound 2 (77 mg, 0.34 mmol) in dioxane/H₂O (1 mL/0.1 mL) was added Pd(dppf)Cl₂ (12 mg, 0.023 mmol) followed by K₂CO₃ (95 mg, 0.69 mmol) at room temperature. The reaction mixture was heated to 100° C. for 30 min by microwave. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by Prep-HPLC to afford compound 170-1 (47 mg, 38%) as a white solid. LC-MS: m/z=530.1 [M+H]⁺ at RT 4.47 (96.79% purity). ¹H NMR (400 MHz, DMSO-d6) δ 11.92 (s, 1H), 7.86 (dd, J=9.5, 2.9 Hz, 1H), 7.76 (s, 1H), 7.61 (s, 1H), 7.53 (dd, J=7.1, 4.6 Hz, 2H), 7.45 (dd, J=8.5, 5.5 Hz, 1H), 7.34-7.19 (m, 2H), 7.12 (t, J=8.9 Hz, 1H), 6.44 (dd, J=9.6, 3.1 Hz, 1H), 4.78 (s, 1H), 4.65 (dd, J=16.7, 10.1 Hz, 1H), 4.52-4.26 (m, 2H), 3.80-3.75 (m, 0.5H), 3.39-3.28 (s, 0.5H), 3.23-3.04 (m, 2H), 2.77-2.54 (m, 2H), 1.26-1.11 (m, 3H), 1.02-0.91 (m, 1H), 0.54-0.11 (m, 4H).

### Example 171

### (S)-2-(tert-Butoxycarbonylamino)propanoic acid (1)

To a stirring solution of SM1 (222 mg, 2.49 mmol) in THF (2 mL), H₂O (2 mL) was added (Boc)₂O (654 mg, 3.0 mmol) followed by NaOH (249.2 mg, 6.23 mmol) at room temperature. The reaction mixture was stirred at room temperature overnight. After consumption of the starting material (by TLC), the reaction mixture was neutralized by 2N HCl, extracted with EtOAc, washed by brine. Combined organic extracts were dried over anhydrous Na₂SO₄ and concentrated under reduced pressure to obtain crude compound 1 (468 mg, 99%) as a clear oil. LC-MS: m/z=212 [M+Na]⁺

### tert-Butyl (S)-1-((S)-1-(2-(benzyl((S)-1-cyclopropylethyl)amino)-2-oxoethyl)-2,5-dioxo-2′,3′-dihydrospiro[imidazolidine-4,1′-indene]-5′-ylamino)-1-oxopropan-2-ylcarbamate (2)

To a stirring solution of compound 1 (20 mg, 0.106 mmol) in THF (0.5 mL) was added NMM (21.4 mg, 0.212 mmol) followed by isobutyl chloroformate (14.4 mg, 0.106 mmol) at −15° C. and stirred at −15° C. for 20 min. After consumption of the starting material (by TLC), Intermediate 33 (48 mg, 0.111 mmol) in THF (3.5 mL) was added at room temperature and the mixture was stirred at room temperature for 3 hour and concentrated under reduced pressure. The residue was dissolved in EtOAc, washed by 10% Na₂CO₃ solution, 0.1 M HCl, brine, combined the organic layers were dried over anhydrous Na₂SO₄ and concentrated under reduced pressure to obtain crude compound 2 (63 mg, 99%) as a white solid. LC-MS: m/z=604 [M+H-Boc]⁺

To a stirring solution of compound 2 (63 mg, 0.104 mmol) in DCM (1 mL) was added TFA (1 mL) at room temperature and stirred at room temperature for 0.5 hour. After consumption of the starting material (by TLC), the reaction mixture was concentrated under reduced pressure to obtain crude product, which was purified by silica gel column chromatography eluting with 5% MeOH/DCM afford 171-1 (52 mg, 81%) as a white solid. LC-MS: m/z=504.2 [M+H]⁺. ¹H NMR (400 MHz, MeOD): δ ¹H NMR (400 MHz, MeOD) δ 7.70 (s, 1H), 7.49-7.17 (m, 7H), 4.83-4.66 (m, 2H), 4.53-4.32 (m, 2H), 4.05 (dd, J=21.8, 14.0 Hz, 1H), 3.88-3.76 (m, 1H), 3.14 (dt, J=13.5, 6.8 Hz, 2H), 2.80-2.68 (m, 1H), 2.41-2.29 (m, 1H), 1.61 (dd, J=7.1, 1.9 Hz, 3H), 1.32-1.21 (m, 3H), 1.01-0.96 (m, 1H), 0.62-0.51 (m, 1H), 0.42-0.21 (m, 3H).

### Example 172

### 1-Aminocyclopropanecarbonyl chloride hydrochloride (1)

To a stirring solution of ozazolidin-2-one (0.922 g, 10.6 mmol) in MeCN (20 mL) was added PCl₅ (2.95 g, 14.2 mmol) at room temperature. The reaction mixture was stirred at room temperature overnight. Then SM1 (1 g, 8.7 mmol) was added and the reaction mixture was stirred at room temperature for 8 h and filtered. The residue was washed by MeCN, dried to obtain compound 1 (1.24 g, 84%) as a white solid.

To a stirring solution of compound 1 (40 mg, 0.212 mmol) in DCM (0.5 mL) was added intermediate 18 (46 mg, 0.106 mmol), followed by Py (0.5 mL) at room temperature and stirred at room temperature overnight. After consumption of the starting material (by TLC), the reaction mixture was concentrated under reduced pressure to obtain crude product, which was purified by prep-TLC eluting with 5% MeOH/DCM afford 172-1 (35 mg, 62%) as a white solid. LC-MS: m/z=531.3 [M+H]⁺. ¹H NMR (400 MHz, MeOD): δ 7.78 (s, 1H), 7.57-7.19 (m, 7H), 4.85-4.65 (m, 2H), 4.63-4.39 (m, 2H), 3.92-3.81 (m, 1H), 3.30-3.21 (m, 1H), 3.20-3.07 (m, 1H), 2.88-2.79 (m, 1H), 2.77-2.68 (m, 2H), 2.60 (tdd, J=12.5, 8.2, 4.0 Hz, 1H), 2.18-1.98 (m, 4H), 1.37-1.25 (m, 3H), 0.99 (d, J=5.7 Hz, 1H), 0.68-0.52 (m, 1H), 0.48-0.23 (m, 3H).

### Example 173

### N-benzyl-N—((S)-1-cyclopropylethyl)-2-(5′-(furan-2-yl)-2,5-dioxo-2′,3′-dihydrospiro[imidazolidine-4,1′-indene]-1-yl)acetamide (1)

To a stirring solution of SM1 (150 mg, 0.302 mmol) in dioxane (4.5 mL), H₂O (1.5 mL) was added furan-2-ylboronic acid (101 mg, 0.906 mmol), and NaHCO₃ (127 mg, 1.51 mmol) followed by Pd(dppf)Cl₂ (11 mg, 0.0151 mmol) at room temperature. The reaction mixture was stirred under microwave irradiation at 100° C. under N₂ for 30 minutes. EtOAc was added and then filtered through a short silica gel column. The filtrate was concentrated under reduced pressure to obtain crude product, which was purified by silica gel column chromatography eluting with 100%-95% DCM/methanol to afford compound 1 (140 mg, 95%) as an yellow solid. LC-MS: m/z=484 [M+H]⁺

To a stirring solution of compound 1 (48 mg, 0.1 mmol) in t-butanol (0.4 mL) was added water (0.1 mL) and NaH₂PO₄ (18 mg, 0.15 mmol) followed by NaClO₂ (28 mg, 0.3 mmol)) at room temperature and stirred at room temperature for 2 hours. The reaction mixture was concentrated under reduced pressure to obtain crude product, which was purified by prep-HPLC eluting with ACN/H₂O containing 5% TFA to afford 173-1 (15 mg, 29%) as a white solid. LC-MS: m/z=516.2 [M+H] at RT 4.163 (93.809% purity). ¹H NMR (301 MHz, CD3OD): δ 7.90 (t, J=19.2 Hz, 2H), 7.60-6.88 (m, 7H), 6.39-6.07 (m, 1H), 4.82-4.21 (m, 4H), 3.81 (s, 0.6H), 3.34 (s, 0.4H), 3.20 (s, 2H), 2.76 (s, 1H), 2.38 (dd, J=13.6, 6.4 Hz, 1H), 1.44-1.12 (m, 3H), 0.94 (s, 1H), 0.59 (s, 1H), 0.33 (dd, J=17.1, 12.3 Hz, 3H).

### Example 174

### 2-cyanoacetyl chloride (1)

To a mixture of 2-cyanoacetic acid SM1 (21 mg, 0.24 mmol) in DCM (5 mL) was added oxalyl dichloride (34 mg, 0.27 mmol) and DMF (0.05 ml), then stirred at room temperature under N₂ for 0.5 h. After consumption of the starting material (by TLC), the solvent was removed under reduce pressure to afford 1 (25 mg, 100%) as white solid which used directly without further purification with next step.

To a solution of 2-cyanoacetyl chloride 1 (25 mg, 0.24 mmol) in DCM (10 mL) was added 2-(5-amino-2′,4′-dioxo-2,3-dihydrospiro[indene-1,5′-ozazolidine]-3′-yl)-N—((S)-1-cyclopropylethyl)-N-(4-fluorobenzyl)acetamide 3 (100 mg, 0.22 mmol) and Et₃N, then stirred at room temperature under N₂ for 1 hour. After consumption of the starting material (by TLC) diluted with water and extracted with EtOAc. Combined organic extracts were dried over anhydrous MgSO₄ and concentrated under reduced pressure to obtain crude product, which was purified by silica gel column chromatography eluting with 40% EtOAc/PE to afford 174-1 (75 mg, 66%) as white solid. LC-MS: m/z=519.2 [M+H]⁺ at RT 4.75 (95.27% purity). ¹H NMR (400 MHz, DMSO-d6) δ 10.49 (s, 1H), 7.71 (d, J=5.6 Hz, 1H), 7.40 (m, 3H), 7.26 (m, 2H), 7.12 (m, 1H), 4.78 (s, 1H), 4.64 (m, 1H), 4.53-4.23 (m, 2H), 3.94 (s, 2H), 3.76 m, 1H), 3.29-2.96 (m, 3H), 2.66 (m, 1H), 2.54 (s, 1H), 1.27-1.08 (m, 3H), 0.93 (m, 1H), 0.59-0.30 (m, 2H), 0.21 (m, 2H).

### Example 175

### (S)-1-cyclopropyl-N-(4-fluorobenzyl)ethanamine (1)

To a stirring mixture of SM1 (1.635 g, 13.176 mmol), (S)-1-cyclopropylethanamine (0.935 g, 11 mmol) in MeOH (50 mL) was added NaBH(OAc)₃ (4.664 g, 22 mmol) at room temperature. The reaction mixture was stirred at room temperature overnight. After consumption of the starting material (by TLC), the reaction mixture was diluted with water and extracted with EtOAc. Combined organic extracts were dried over anhydrous Na₂SO₄ and concentrated under reduced pressure to obtain the crude product, which was purified by silica gel column chromatography eluting with 5% methanol/DCM to afford compound 1 (858 mg, 75%) as a colorless oil. LC-MS: m/z=194 [M+H]⁺.

### (S)-2-bromo-N-(1-cyclopropylethyl)-N-(4-fluorobenzyl)acetamide (2)

To a stirring solution of compound 1 (858 mg, 4.45 mmol in DCM (30 mL) was added pyridine (352 mg, 4.45 mmol) followed by 2-bromoacetyl bromide (898 mg, 4.45) at 0-5° C. The reaction mixture was stirred at room temperature for 2 hours. After consumption of the starting material (by TLC), the reaction mixture was diluted with water and extracted with EtOAc. Combined organic extracts were dried over anhydrous Na₂SO₄ and concentrated under reduced pressure to obtain the crude product, which was purified by silica gel column chromatography eluting with 100% DCM to afford compound 2 (1.157 g, 83%) as a yellow oil. LC-MS: m/z=314/316 [M+H]⁺

### 2-(5-acetamido-2′,4′-dioxo-2,3-dihydrospiro[indene-1,5′-ozazolidine]-3′-yl)-N—((S)-1-cyclopropylethyl)-N-(4-fluorobenzyl)acetamide (3)

To a stirring solution of N-(2′,4′-dioxo-2,3-dihydrospiro[indene-1,5′-ozazolidine]-5-yl)acetamide (497 mg, 1.91 mmol) in DMF (10 mL) was added K₂CO₃ (527 mg, 3.82 mmol) followed by compound 2 (600 mg, 1.91 mmol) at room temperature and stirred at room temperature overnight. The reaction mixture was diluted with water and extracted with EtOAc. Combined organic extracts were dried over anhydrous Na₂SO₄ and concentrated under reduced pressure to obtain the crude product, which was purified by silica gel column chromatography eluting with 5% methanol/DCM to afford compound 3 (671 mg, 71%) as a brown solid. LC-MS: m/z=494 [M+H]⁺

### 2-(5-amino-2′,4′-dioxo-2,3-dihydrospiro[indene-1,5′-ozazolidine]-3′-yl)-N—((S)-1-cyclopropylethyl)-N-(4-fluorobenzyl)acetamide (4)

To a stirring solution of compound 3 (671 mg, 1.361 mmol) in methanol (10 mL) was added concentrated HCl (3 mL) followed by water (5 mL) at room temperature and stirred at 60° C. for 5 hours. Most solvent was removed under reduced pressure and the resulting mixture was basified with sat. NaHCO₃ to pH=8-10 and extracted with EtOAc. Combined organic extracts were dried over anhydrous Na₂SO₄ and concentrated under reduced pressure to obtain the crude product, which was purified by silica gel column chromatography eluting with 5% methanol/DCM to afford compound 4 (517 mg, 84%) as a brown solid. LC-MS: m/z 452 [M+H]⁺

To a stirring solution of compound 4 (100 mg, 0.221 mmol) in DCM (5 mL) was added triphosgene (66 mg, 0.221 mmol) followed by Et₃N (67 mg, 0.663 mmol) at room temperature and stirred at room temperature for 5 hours. The solution of methanamine in THF (2 M, 0.33 mL) was added and then the resulting mixture was stirred for 1 hour. The solvent was removed under reduced pressure and purified by prep-HPLC eluting with ACN/H₂O containing 5% TFA to afford 175-1 (43 mg, 38%) as a white solid. LC-MS: m/z=509.1 [M+H]⁺ at RT 4.770 (100% purity). ¹H NMR (400 MHz, CD3OD): δ 7.62-6.92 (m, 7H), 4.83-4.32 (m, 4H), 3.84 (dd, J=11.9, 9.0 Hz, 0.6H), 3.35 (s, 0.4H), 3.26-3.14 (m, 1H), 3.14-3.02 (m, 1H), 2.87-2.68 (m, 4H), 2.57 (td, J=14.1, 7.1 Hz, 1H), 1.29 (dd, J=34.5, 6.6 Hz, 3H), 0.98 (d, J=4.7 Hz, 1H), 0.64-0.53 (m, 1H), 0.43-0.15 (m, 3H).

### Example 176

To a solution of intermediate 27 (50 mg, 0.1 mmol) and azetidin-3-ylmethanamine (18 mg, 0.2 mmol) in DMSO (3 ml) was added K₃PO₄ (127 mg, 0.6 mmol), CuI (21 mg, 0.11 mmol) and L-proline (13 mg, 0.11 mmol) under N₂. Then stirred at 90° C. for 16 hours. After consumption of the starting material (by TLC), diluted with water and extracted with EtOAc. Combined organic extracts were dried over anhydrous MgSO₄ and concentrated under reduced pressure to obtain crude product, which was purified by Prep-HPLC (MeCN/H₂O 3:2) to afford 176-1 (10 mg, 20%) as yellow solid. LC-MS: m/z=502.2 [M+H]⁺ at RT 3.58 (99.59% purity). ¹H NMR (400 MHz, CD₃OD) δ 7.71-6.92 (m, 6H), 6.61 (d, J=11.3 Hz, 1H), 6.46-6.32 (m, 1H), 4.84-4.67 (m, 2H), 4.59-4.37 (m, 1H), 4.32 (dd, J=14.8, 10.6 Hz, 1H), 4.01 (t, J=7.6 Hz, 1H), 3.82 (dd, J=9.7, 6.8 Hz, 1H), 3.66 (t, J=6.2 Hz, 1H), 3.38 (d, J=6.0 Hz, 1H), 3.28 (d, J=7.4 Hz, 2H), 3.14 (dd, J=11.9, 6.8 Hz, 1H), 3.06 (t, J=14.3 Hz, 2H), 2.99 (d, J=5.3 Hz, 1H), 2.67 (td, J=12.5, 6.0 Hz, 1H), 2.37-2.18 (m, 1H), 1.35-1.18 (m, 3H), 1.01-0.89 (m, 1H), 0.59 (tdd, J=14.1, 9.0, 5.3 Hz, 1H), 0.45-0.18 (m, 3H).

### Example 177

To a stirring solution of intermediate 28 (40 mg, 0.04 mmol) in dioxane/H₂O (10 mL) was added K₂CO₃ (10 mg, 0.08 mmol), dppf PdCl₂ (5.3 mg, 0.004 mmol) and 1H-pyrazol-4-ylboronic acid (6.8 mg, 0.06 mmol) and refluxing under N2 for 2 hours. After consumption of the starting material (by TLC), the mixture was diluted with water and extracted with DCM, Combined organic extracts were dried over anhydrous Na₂SO₄ and concentrated under reduced pressure to obtain crude product, which was purified by silica gel column chromatography to afford 177-1 (16 mg, 45%) as an off-white solid. LC-MS: m/z=484.22 [M+H]⁺ at RT 1.61 (100% purity). ¹H NMR (400 MHz, DMSO) δ 12.98 (s, 1H), 7.77 (dd, J=50.8, 20.6 Hz, 3H), 7.52 (d, J=7.8 Hz, 2H), 7.41 (s, 2H), 7.26 (t, J=18.6 Hz, 2H), 6.78 (s, 1H), 4.89-4.60 (m, 2H), 4.55-4.18 (m, 2H), 3.79 (s, 1H), 3.30-3.02 (m, 2H), 2.70 (d, J=8.2 Hz, 1H), 2.33 (s, 1H), 1.18 (dd, J=26.6, 6.0 Hz, 3H), 0.98 (s, 1H), 0.50 (s, 1H), 0.28 (t, J=37.3 Hz, 3H).

### Example 178

### 2-(tert-butoxycarbonylamino)-3-hydroxypropanoic acid (1)

A mixture of compound 1 (500 mg, 4.75 mmol), NaOH (200 mg, 4.75 mmol), Et₃N (500 mg, 4.75 mmol) and (Boc)₂O (1 g, 4.75 mmol) in MeOH:H₂O (1:1, 10 mL) was stirred at room temperature overnight. After consumption of the starting material (by TLC), the solvent was remove to afford to give compound 2 (1 g, 90%) as a white solid. LC-MS: m/z 106 [M+H]⁺

### tert-butyl 1-(3′-(2-(benzyl((S)-1-cyclopropylethyl)amino)-2-oxoethyl)-2′,4′-dioxo-2,3-dihydrospiro[indene-1,5′-ozazolidine]-5-ylamino)-3-hydroxy-1-oxopropan-2-ylcarbamate (2)

To a cold solution of compound 2 (36 g, 0.1755 mmol) and NMM (35.5 mg, 0.351 mmol) in anhydrous THF (3 mL) was added drop-wise isobutyl carbonochloridate (24 mg, 0.1755 mmol) at 0° C. under N₂. The mixture was stirred at room temperature for 15 mins. After the mixture was stirred for another 15 mins, compound 4 (50 mg, 3.9 mmol) was added. Then the mixture was stirred at room temperature overnight. The reaction mixture was diluted with water and extracted with EtOAc. Combined organic extracts were dried over anhydrous Na₂SO₄ and concentrated under reduced pressure to obtain crude product, The crude product was Prep-HPLC to afford compound 5 (14 mg, 20%) as a white solid. LC-MS: m/z 620.6 [M+H]⁺

### 2-amino-N-(3′-(2-(benzyl((S)-1-cyclopropylethyl)amino)-2-oxoethyl)-4′-oxo-2,3-dihydrospiro[indene-1,5′-ozazolidine]-5-yl)-3-hydroxypropanamide hydrochloride (3)

A mixture of compound 5 (14 mg, 0.0226 mmol) and 5N HCl (3 mL) was stirred a at room temperature for 2 hours. After consumption of the starting material (by TLC), the reaction mixture was concentrated under reduced pressure. The solvent was remove to afford to give compound 5 (10 mg, 90%) as a white solid. LC-MS: m/z=520.6 [M+H]⁺

A mixture of compound 5 (14 mg, 0.0226 mmol) and 5N HCl (3 mL) was stirred a at room temperature for 2 hours. After consumption of the starting material (by TLC), the reaction mixture was concentrated under reduced pressure. The solvent was removed to afford to give 178-1 HCl salt (10 mg, 90%) as a white solid. LC-MS: m/z=520.23 [M+H]. ¹H NMR (400 MHz, MeOD-d6): δ 7.75 (s, 1H), 7.53-7.38 (m, 1H), 7.36-7.20 (m, 1H), 4.86-4.73 (m, 1H), 4.73-4.47 (m, 1H), 4.50-4.39 (m, 1H), 4.12 (d, J=3.3 Hz, 1H), 4.04 (dt, J=14.5, 7.3 Hz, 1H), 3.98 (dd, J=10.1, 7.3 Hz, 1H), 3.91-3.76 (m, 1H), 3.25 (dd, J=15.8, 7.4 Hz, 1H), 3.19-3.05 (m, 1H), 2.89-2.73 (m, 1H), 2.58 (ddd, J=14.2, 10.5, 6.4 Hz, 1H), 1.32 (d, J=6.1 Hz, 1H), 1.25 (d, J=6.8 Hz, 1H), 1.08-0.87 (m, 1H), 0.72-0.49 (m, 1H), 0.33 (dd, J=26.5, 19.6 Hz, 1H).

### Example 179

### (S)—N-(3′-(2-(benzyl(1-cyclopropylethyl)amino)-2-oxoethyl)-2′,4′-dioxo-2,3-dihydrospiro[indene-1,5′-ozazolidine]-5-yl)-2-hydroxy-2-methylpropanamide

To a stirring solution of compound Intermediate-18 (43 mg, 0.1 mmol) in DMF (1 mL) was added compound SM1 (11 mg, 0.11 mmol) and DIPEA (40 mg, 0.3 mmol) at room temperature. To this added HATU (46 mg, 0.12 mmol) at room temperature and the reaction mixture was stirred at room temperature for 4 days. The reaction mixture was diluted with water and extracted with EtOAc. Combined organic extracts were dried over anhydrous Na₂SO₄ and concentrated under reduced pressure to obtain crude product, which was purified by Pre-TLC eluting with 66% EtOAc/Hexane to afford compound 179-1 (12 mg, 23%). as an off-white solid. TLC: 66% EtOAc/Hexane (Rf: 0.5). LC-MS: m/z=520 [M+H]⁺ (98% purity). ¹H NMR (400 MHz, CD₃OD-d4) δ 7.75 (s, 1H), 7.52-7.16 (m, 7H), 4.85-4.74 (m, 2H), 4.70-4.50 (m, 1H), 4.44 (s, 1H), 3.82 (s, 1H), 3.14 (s, 1H), 2.78 (s, 1H), 2.59 (s, 1H), 1.46 (s, 6H), 1.28 (dd, J=21.7, 7.0 Hz, 3H), 0.98 (s, 1H), 0.59 (s, 1H), 0.29 (s, 3H).

### Example 180

### 1-(tert-Butoxycarbonylamino)cyclopropanecarboxylic acid (1)

To a stirring solution of SM1 (202 mg, 2.0 mmol) in THF (4 mL), H₂O (1 mL) was added (Boc)₂O (523 mg, 2.4 mmol) followed by NaOH (200 mg, 5 mmol) at room temperature. The reaction mixture was stirred at room temperature overnight. After consumption of the starting material (by TLC), the reaction mixture was neutralized by 2N HCl, extracted with EtOAc, washed by brine. Combined organic extracts were dried over anhydrous Na₂SO₄ and concentrated under reduced pressure to obtain crude compound 1 (335 mg, 83%) as a white solid. LC-MS: m/z=146 [M+H-tBu]⁺

### tert-Butyl 1-(3′-(2-(benzyl((S)-1-cyclopropylethyl)amino)-2-oxoethyl)-2′,4′-dioxo-2,3-dihydrospiro[indene-1,5′-ozazolidine]-5-ylcarbamoyl)cyclopropylcarbamate (2)

To a stirring solution of compound 1 (23 mg, 0.115 mmol) in THF (2 mL) was added NMM (23 mg, 0.23 mmol) followed by isobutyl carbonochloridate (16 mg, 0.115 mmol) at −15° C. and stirred at −15° C. for 20 min. After consumption of the starting material (by TLC), Intermediate 18 (50 mg, 0.115 mmol) was added at room temperature and the mixture was stirred at room temperature for 3 hours and concentrated under reduced pressure. The residue was dissolved in EtOAc, washed by 10% Na₂CO₃ solution, 0.1 M HCl, brine, combined the organic layers were dried over anhydrous Na₂SO₄ and concentrated under reduced pressure to obtain crude compound 2 (70 mg, 99%) as a yellow oil. LC-MS: m/z=561 [M+H-t-Bu]⁺

To a stirring solution of compound 2 (70 mg, 0.114 mmol) in DCM (1 mL) was added TFA (1 mL) at room temperature and stirred at room temperature for 0.5 h. After consumption of the starting material (by TLC), the reaction mixture was concentrated under reduced pressure to obtain crude product, which was purified by prep-TLC eluting with 5% MeOH/DCM afford 180-1 (46 mg, 78%) as a white solid. LC-MS: m/z=517.2 [M+H]⁺. ¹H NMR (400 MHz, MeOD): δ ¹H NMR (400 MHz, MeOD) δ 7.74 (s, 1H), 7.52-7.19 (m, 7H), 4.77 (dd, J=28.1, 22.4 Hz, 2H), 4.64-4.45 (m, 2H), 3.85 (s, 1H), 3.26-3.20 (m, 1H), 3.17-3.10 (m, 1H), 2.80 (dd, J=13.4, 7.5 Hz, 1H), 2.59 (dd, J=8.5, 4.2 Hz, 1H), 1.52 (s, 2H), 1.34-1.24 (m, 3H), 1.17 (s, 2H), 0.97 (s, 1H), 0.61 (dd, J=13.0, 8.5 Hz, 1H), 0.43-0.26 (m, 3H).

### Example 181

To a stirring solution of SM1 (80 mg, 0.177 mmol) in DCM (1.5 mL) was added pyridine (1.5 mL) followed by 2-amino-2-methylpropanoyl chloride hydrochloride (68 mg, 0.424 mmol) at room temperature and stirred at room temperature overnight. The solvent was removed under reduced pressure and purified by prep-TLC developing with 10% methanol in DCM to afford 181-1 (35 mg, 37%) as a white solid. LC-MS: m/z=537.2 [M+H]⁺ at RT 3.985 (99.196% purity). ¹H NMR (301 MHz, CD₃OD): δ 7.96-6.80 (m, 7H), 4.85-4.29 (m, 4H), 3.83 (s, 0.6H), 3.35 (s, 0.4H), 3.27-2.98 (m, 2H), 2.79 (dt, J=15.1, 8.4 Hz, 1H), 2.69-2.47 (m, 1H), 1.80-1.08 (m, 9H), 0.90 (d, J=7.3 Hz, 1H), 0.75-0.21 (m, 4H).

### Example 182

### (S)-tert-butyl 3-(3′-(2-(benzyl(1-cyclopropylethyl)amino)-2-oxoethyl)-2′,4′-dioxo-2,3-dihydrospiro[indene-1,5′-ozazolidine]-5-ylamino)azetidine-1-carboxylate (compound 1)

To a mixture of tert-butyl 3-aminoazetidine-1-carboxylate SM (258 mg, 1.5 mmol), (S)—N-benzyl-2-(5-bromo-2′,4′-dioxo-2,3-dihydrospiro[indene-1,5′-ozazolidine]-3′-yl)-N-(1-cyclopropylethyl)acetamide Inter-28 (72 mg, 0.15 mmol) and Cs₂CO₃ (54 mg, 0.17 mmol) in toluene (2 mL) was added BINAP (4 mg, 0.007 mmol) and Pd(OAc)₂ (1 mg, 0.005 mmol) under argon atmosphere. The mixture was degassed and sealed, which was stirred at 100° C. for 3 hours under microwave. The reaction mixture was diluted with ethyl acetate (30 mL) and then filtered. The filtrate was concentrated under reduced pressure to obtain crude product, which was purified by column chromatography (petroleum ether:ethyl acetate=2:1) to afford (S)-tert-butyl 3-(3′-(2-(benzyl(1-cyclopropylethyl)amino)-2-oxoethyl)-2′,4′-dioxo-2,3-dihydrospiro[indene-1,5′-ozazolidine]-5-ylamino)azetidine-1-carboxylate compound 1 (10 mg). LC-MS: m/z=489.2 [M+H-Boc]⁺

### (S)-2-(5-(azetidin-3-ylamino)-2′,4′-dioxo-2,3-dihydrospiro[indene-1,5′-ozazolidine]-3′-yl)-N-benzyl-N-(1-cyclopropylethyl)acetamide

To a solution of compound 1 in dichloromethane (6 mL) was added trifluoroacetic acid (2 mL), the mixture was stirred at room temperature for 1 hour. The reaction mixture was evaporated to dryness under reduced pressure to obtain crude product, which was purified by Prep-HPLC to afford 182-1 as a TFA salt (3.0 mg, 4% two steps) as off-white solid. LC-MS: m/z=489.2 [M+H]⁺ at RT 3.96 (93.13% purity). ¹H NMR (400 MHz, CD₃OD-d4): δ 7.48-7.29 (m, 5H), 6.58-6.54 (m, 2H), 4.85-4.34 (m, 6H), 4.02-3.94 (m, 2H), 3.86-3.82 (m, 1H), 3.24-3.14 (m, 1H), 3.05-2.98 (m, 1H), 2.77-2.71 (m, 2H), 2.58-2.50 (m, 2H), 1.35-1.27 (m, 3H), 1.02-0.96 (m, 1H), 0.63-0.95 (m, 1H), 0.47-0.22 (m, 3H).

### Example 183

### N-benzyl-N—((S)-1-cyclopropylethyl)-2-((S)-5′-(diphenylmethyleneamino)-2,5-dioxo-2′,3′-dihydrospiro[imidazolidine-4,1′-indene]-1-yl)acetamide (1)

To a stirring solution of Pd₂(dba)₃ (202 mg, 0.22 mmol) and BINAP (274 mg, 0.44 mmol) in toluene (20 mL) was added Intermediate-32 (1.1 g, 2.22 mmol) and diphenylmethanimine (804 mg, 4.44 mmol) followed by t-BuONa (640 mg, 6.66 mmol) at 100° C. The reaction mixture was heated to 100° C. for 3 hours. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 50% EtOAc/PE to afford compound 1 (1.09 g, 82%) as a yellow solid. LC-MS: m/z=597.2 [M+H]⁺

### 2-((S)-5′-amino-2,5-dioxo-2′,3′-dihydrospiro[imidazolidine-4,1′-indene]-1-yl)-N-benzyl-N—((S)-1-cyclopropylethyl)acetamide (2)

To a stirring solution of compound 1 (1.02 g, 1.71 mmol) in THF (10 mL) was added 1N HCl_{aq} (3.5 mL) at room temperature. The reaction mixture was stirred at room temperature for 1.5 hours. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 50% CH₃OH/CH₂Cl₂ to afford compound 2 (650 mg, 88%) as a yellow solid. LC-MS: m/z=433.2 [M+H]⁺

To a stirring solution of compound 2 (100 mg, 0.23 mmol) and compound 3 (39 mg, 0.23 mmol) in DCM (6 mL) was added Et3N (70 mg, 0.69 mmol) at room temperature. The reaction mixture was stirred at room temperature for 16 hours. After consumption of the starting material (by TLC), the reaction mixture was quenched with water. The mixture was concentrated under reduced pressure to obtain crude product, which was purified by Prep-HPLC to afford compound 183-1 (52 mg, 43%) as a white solid. LC-MS: m/z=530.3 [M+H]⁺ at RT 3.57 (100% purity). ¹H NMR (300 MHz, DMSO-d6) δ 10.16 (s, 1H), 8.79 (d, J=13.9 Hz, 1H), 8.53 (s, 2H), 7.60 (s, 1H), 7.52-7.43 (m, 1H), 7.41-7.06 (m, 6H), 4.74 (s, 1H), 4.61 (s, 1H), 4.35 (d, J=23.2 Hz, 1H), 4.18 (d, J=2.7 Hz, 1H), 3.75-3.63 (m, 0.5H), 3.39-3.28 (m, 0.5H), 3.09-2.96 (m, 2H), 2.80 (s, 1H), 2.59-2.52 (m, 1H), 2.34-1.98 (m, 6H), 1.14 (dd, J=24.9, 6.5 Hz, 3H), 1.02-0.88 (m, 1H), 0.47-0.13 (m, 4H).

### Example 184

### (R)-2-(tert-butoxycarbonylamino)propanoic acid (2)

To a stirring solution of (R)-2-aminopropanoic acid SM1 (0.2 g, 2.25 mmol) in THF (10 mL) was added (Boc)₂O (0.58 g, 2.7 mmol) and 10% NaOH aq (10 mL), the mixture was stirred at room temperature for 30 mins. The solvent was removed out in vacuo, the residue was diluted with brine and extracted with EtOAc. Combined organic extracts were dried over anhydrous Na₂SO₄ and concentrated under reduced pressure to obtain crude product as an off-white solid, which was used directly for subsequent step. ¹H NMR (300 MHz, DMSO-d₆): δ 12.44 (s, 1H), 7.11 (s, 1H), 3.91 (m, 1H), 1.47 (d, J=3.0 Hz, 3H), 1.37 (s, 9H).

### tert-butyl (2R)-1-(3′-(2-(benzyl((S)-1-cyclopropylethyl)amino)-2-oxoethyl)-2′,4′-dioxo-2,3-dihydrospiro[indene-1,5′-ozazolidine]-5-ylamino)-1-oxopropan-2-ylcarbamate (5)

To a cold solution (−15 degree) of SM2 (22 mg, 0.115 mmol), and 4-methyl morpholine in dry THF (5 mL), was added dropwise Isobutyl chloroformate SM3 (16 mg, 0.115 mmol) in dry THF (2 mL) over 15 mins. After the mixture was stirred for another 15 mins, SM4 (50 mg, 0.115 mmol) was added. Then the mixture was allowed to slowly warm to room temperature and stirred overnight. After the evaporation of the solvent in vacuo, the residue was extracted with EA (20 mL), washed with brine (15 mL), dried over MgSO₄. The concentrated organic phase crude was purified by silica gel column chromatography to afford compound 5 (0.03 g, 43%) as an off-white solid. TLC: 50% PE/EA. LC-MS: m/z=605 [M+H]⁺

### (2R)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)propanamide

A solution of SM5 (0.03 g, 0.05 mmol), TFA (2 mL) and DCM (2 mL) was stirred for 2 hours. After consumption of the starting material (by TLC), and the solvent was evaporated off in vacuo. The crude was purified by Prep-HPLC [H₂O-ACN(0.05% TFA)] to afford compound 184-1 (23 mg, 90%) as a white liquid. ¹H NMR (400 MHz, DMSO-d₆): δ 10.68 (d, J=4.9 Hz, 1H), 8.27 (s, 2H), 7.72 (d, J=6.4 Hz, 1H), 7.51-7.23 (m, 7H), 4.80-4.37 (m, 4H), 4.03 (d, J=7.0 Hz, 1H), 3.78 (d, J=6.9 Hz, 1H), 3.66-3.28 (m, 1H), 3.23-3.05 (m, 2H), 2.67 (dd, J=14.6, 6.7 Hz, 1H), 1.46 (dd, J=6.9, 3.0 Hz, 3H), 1.18 (ddd, J=9.5, 8.8, 4.1 Hz, 3H), 1.01-0.93 (m, 1H), 0.53-0.18 (m, 4H). LC-MS: m/z=505.2 [M+H]⁺ (98.99% purity, 214 nm)

### Example 185

To a solution of intermediate-35 (85 mg, 0.2 mmol) and 2-bromothiazole SM1 (49 mg, 0.3 mmol) in Isopropanol (10 mL) was added p-toluenesulfonic acid (34 mg, 0.2 mmol) under N₂. Then stirred at 80° C. for 48 hours. After consumption of the starting material (by TLC), diluted with water and extracted with EtOAc. Combined organic extracts were dried over anhydrous MgSO₄ and concentrated under reduced pressure to obtain crude product, which was purified by Prep-HPLC (MeCN/H₂O 3:2) to afford 185-1 (44 mg, 45%) as yellow solid. LC-MS: m/z=516.2 [M+H]⁺ at RT 4.11 (97.94% purity). ¹H NMR (400 MHz, DMSO-d6) δ 10.29 (s, 1H), 8.73 (d, J=16.4 Hz, 1H), 7.74 (d, J=5.2 Hz, 1H), 7.49-7.11 (m, 8H), 6.92 (m, 1H), 4.75 (s, 1H), 4.62 (s, 1H), 4.48-4.22 (m, 2H), 4.19 (d, J=4.4 Hz, 1H), 3.74 (s, 1H), 3.01 (t, J=6.2 Hz, 2H), 2.56 (m, 1H), 2.28-2.13 (m, 1H), 1.22-1.05 (m, 3H), 0.94 (s, 1H), 0.42 (m, 2H), 0.21 (m, 2H).

### Example 186

### (S)-tert-butyl 3-(1-(2-(benzyl(1-cyclopropylethyl)amino)-2-oxoethyl)-2,5-dioxo-2′,3′-dihydrospiro[imidazolidine-4,1′-indene]-5′-ylamino)azetidine-1-carboxylate (compound 1)

To a mixture of tert-butyl 3-aminoazetidine-1-carboxylate SM (70 mg, 0.4 mmol), (S)—N-benzyl-2-(5′-bromo-2,5-dioxo-2′,3′-dihydrospiro[imidazolidine-4,1′-indene]-1-yl)-N-(1-cyclopropylethyl)acetamide Intermediate-28 (100 mg, 0.2 mmol), L-proline (26 mg, 0.22 mmol), CuI (60 mg, 0.30 mmol) and K₃PO₄ (128 mg, 0.6 mmol) was added DMSO (3 mL). The mixture was degassed and sealed, which was stirred at 100° C. for 1 hour under microwave. The reaction mixture was diluted with ethyl acetate (20 mL) and washed with water (3 mL×3) and then brine (3 mL). The organic extracts was concentrated under reduced pressure to obtain crude product, which was purified by column chromatography (petroleum ether-ethyl acetate=2:1) to afford (S)-tert-butyl 3-(1-(2-(benzyl(1-cyclopropylethyl)amino)-2-oxoethyl)-2,5-dioxo-2′,3′-dihydrospiro[imidazolidine-4,1′-indene]-5′-ylamino)azetidine-1-carboxylate compound 1 (40 mg, 34%) as off-white solid. LC-MS: m/z=609.8 [M+Na]⁺

### (S)-2-(5′-(azetidin-3-ylamino)-2,5-dioxo-2′,3′-dihydrospiro[imidazolidine-4,1′-indene]-1-yl)-N-benzyl-N-(1-cyclopropylethyl)acetamide

To a solution of (S)-tert-butyl 3-(1-(2-(benzyl(1-cyclopropylethyl)amino)-2-oxoethyl)-2,5-dioxo-2′,3′-dihydrospiro[imidazolidine-4,1′-indene]-5′-ylamino)azetidine-1-carboxylate compound 1 (40 mg, 0.07 mmol) in dichloromethane (6 mL) was added trifluroacetic acid (2 mL), the mixture was stirred at room temperature for 1 hour. The reaction mixture was evaporated to dryness under reduced pressure to obtain crude product, which was purified by Prep-HPLC to afford (S)-2-(5′-(azetidin-3-ylamino)-2,5-dioxo-2′,3′-dihydrospiro[imidazolidine-4,1′-indene]-1-yl)-N-benzyl-N-(1-cyclopropylethyl)acetamide 186-1 (4.5 mg, 14%) as an off-white solid. ¹H NMR (400 MHz, CD₃OD-d4): δ 7.52-7.35 (m, 2H), 7.30-7.14 (m, 4H), 6.51-6.48 (m, 2H), 4.87-4.74 (m, 2H), 4.60-4.28 (m, 4H), 4.05-3.91 (m, 2H), 3.85-3.79 (m, 1H), 3.10-3.00 (m, 2H), 2.76-2.62 (m, 1H), 2.32-2.24 (m, 1H), 1.40-1.17 (m, 3H), 1.05-0.91 (m, 1H), 0.68-0.51 (m, 1H), 0.42-0.25 (m, 3H). LC-MS: m/z=488.3 [M+H]⁺ at RT 3.56 (99.35% purity)

### Example 187

### (S)-tert-butyl 2-(3′-(2-(benzyl(1-cyclopropylethyl)amino)-2-oxoethyl)-2′,4′-dioxo-2,3-dihydrospiro[indene-1,5′-ozazolidine]-5-ylcarboxamido)acetate (compound 1)

To a mixture of (S)-3′-(2-(benzyl(1-cyclopropylethyl)amino)-2-oxoethyl)-2′,4′-dioxo-2,3-dihydrospiro[indene-1,5′-ozazolidine]-5-carboxamide (35 mg, 0.08 mmol) in THF (2 mL) was added 60% NaH (7 mg, 0.16 mmol) at 0° C., the mixture was warmed to room temperature and stirred for 10 min, then a solution of tert-butyl 2-bromoacetate SM in THF (0.2 mL) was added by dropwise. The mixture was stirred at room temperature for 30 min. The reaction mixture was quenched with NH₄Cl (sat, aq, 5 mL) and extracted with ethyl acetate (15 mL×2). The organic phase was concentrated to afford a mixture of (S)-tert-butyl 2-(3′-(2-(benzyl(1-cyclopropylethyl)amino)-2-oxoethyl)-2′,4′-dioxo-2,3-dihydrospiro[indene-1,5′-ozazolidine]-5-ylcarboxamido)acetate compound 1 as an off-white solid. LC-MS: m/z=519.7 [M+H-t-Bu]⁺.

### (S)-2-(3′-(2-(benzyl(1-cyclopropylethyl)amino)-2-oxoethyl)-2′,4′-dioxo-2,3-dihydrospiro[indene-1,5′-ozazolidine]-5-ylcarboxamido)acetic acid

To a solution of compound 1 in dichloromethane (6 mL) was added trifluroacetic acid (2 mL), the mixture was stirred at room temperature for 1 hour. The reaction mixture was evaporated to dryness under reduced pressure to obtain crude product, which was purified by Prep-HPLC to afford (S)-2-(3′-(2-(benzyl(1-cyclopropylethyl)amino)-2-oxoethyl)-2′,4′-dioxo-2,3-dihydrospiro[indene-1,5′-ozazolidine]-5-ylcarboxamido)acetic acid 187-1 (8.8 mg, 21%) as off-white solid. ¹H NMR (300 MHz, CD₃OD-d4): δ 7.89 (s, 1H), 7.81 (t, J=8.0 Hz, 1H), 7.65 (d, J=8.0 Hz, 1H), 7.45 (d, J=8.0 Hz, 1H), 7.44 (t, J=8.0 Hz, 1H), 7.35-7.20 (m, 3H), 4.87-4.41 (m, 4H), 4.12 (s, 2H), 3.87-3.83 (m, 1H), 3.30-3.16 (m, 2H), 2.93-2.78 (m, 1H), 2.68-2.59 (m, 1H), 1.34-1.25 (m, 3H), 1.05-0.93 (m, 1H), 0.69-0.53 (m, 1H), 0.46-0.23 (m, 3H). LC-MS: m/z=520.2 [M+H]⁺ at RT 4.25 (99.28% purity).

### Example 188

Compound 188-1 was made according to the above general synthetic procedures.

### Example 189

Compound 189-1 was made according to the above general synthetic procedures.

### Example 190

Compound 190-1 was made from intermediate 20 and intermediate 14 according to the above general synthetic procedures.

### Example 191

Compound 191-1 was made according to the above general synthetic procedures.

### Example 192

Compound 192-1 was made according to the above general synthetic procedures.

### Example 193

### 2-(5′-allyl-2,5-dioxo-2′,3′-dihydrospiro[imidazolidine-4,1′-indene]-1-yl)-N-benzyl-N-(1-cyclopropylethyl)acetamide (1)

To a mixture of intermediate 26 (600 mg, 1.21 mmol) and allyl tributylstannane (801 mg, 2.42 mmol) in dioxane (5 mL) were added CsF (551 mg, 3.63 mmol) and Pd(PPh₃)₄ (140 mg, 0.12 mmol). The resulting mixture was stirred at reflux for 3 h. The reaction was quenched by addition of aqueous NH₄Cl solution and extracted with ethyl acetate. The organic layers were combined, dried over Na₂SO₄, concentrated and purified by silica gel (methanol:DCM=1:20) to give compound 1 (430 mg, 78%) as a white solid. LC-MS: m/z=458.3 [M+H]⁺

### 2-(1-(2-(benzyl(1-cyclopropylethyl)amino)-2-oxoethyl)-2,5-dioxo-2′,3′-dihydrospiro[imidazolidine-4,1′-indene]-5′-yl)acetic acid (2)

To a solution of compound 1 (430 mg, 0.94 mmol) in MeCN (5 mL) and H₂O (5 mL) were added NaIO₄ (601 mg, 2.82 mmol) and RuCl₃ (40 mg, 0.19 mmol). The resulting mixture was stirred at rt for 2 h, then quenched with aqueous solution of NH₄Cl and extracted with ethyl acetate. The combined organic phase was dried over Na₂SO₄ and concentrated to give compound 2 (400 mg, 90%) as a crude solid product. LC-MS: m/z=478.5 [M+H]⁺

### (E)-N-benzyl-N-(1-cyclopropylethyl)-2-(5′-(1-(dimethylamino)-3-oxoprop-1-en-2-yl)-2,5-dioxo-2′,3′-dihydrospiro[imidazolidine-4,1′-indene]-1-yl)acetamide (3)

To a solution of compound 2 (400 mg, 0.84 mmol) in DMF (5 mL) was added POCl₃ (1.5 mL). The resulting solution was stirred at 70° C. for 3 h. The reaction mixture was cooled to room temperature, diluted with water and extracted with ethyl acetate. The combined organic phase was concentrated and purified by silica gel (methanol:DCM=1:20) to give compound 3 (80 mg, 22%) as a solid. LC-MS: m/z=515.5 [M+H]⁺

### N-benzyl-2-(5′-(5-cyano-6-oxo-1,6-dihydropyridin-3-yl)-2,5-dioxo-2′,3′-dihydrospiro[imidazolidine-4,1′-indene]-1-yl)-N-(1-cyclopropylethyl)acetamide

To a solution of compound 3 (80 mg, 0.15 mmol) and 2-cyanoacetamide (26 mg, 0.31 mmol) in MeOH (5 mL) was added MeONa (24.3 mg, 0.45 mmol). The resulting mixture was stirred at reflux for 16 h. After cooling to room temperature, followed by quenching with an aqueous solution of NH₄Cl, the mixture was extracted with ethyl acetate, dried over Na₂SO₄ and purified by Prep-HPLC, eluting with a gradient of CH₃CN/H₂O (containing 0.1% TFA) from 10/90 to 70/30 to give the desired compound 193-1 (20 mg, 21%) as a white solid. LC-MS: m/z=536.2 [M+H]⁺ at RT 4.17 (98.63% purity). ¹H NMR (300 MHz, CD₃OD-d₄) δ 8.46 (s, 1H), 8.00 (s, 1H), 7.51-7.26 (m, 8H), 4.88-4.75 (m, 2H), 4.49-4.25 (m, 2H), 3.81 (m, 1H), 3.35-3.31 (m, 1H), 2.74 (m, 1H), 2.35 (m, 1H), 1.40-1.20 (m, 3H), 0.94 (m, 1H), 0.35-0.22 (m, 4H).

### Example 194

### N-benzylpentan-3-amine (1)

To a mixture of benzylamine (2.5 g, 23 mmol) and pentan-3-one (4.0 g, 46 mmol) in MeOH (25 mL) were added tetraisopropoxytitanium (650 mg, 0.23 mmol) and NaBH₃CN (4.3 g, 69 mmol). The resulting mixture was stirred at room temperature for 3 h. The reaction was quenched by addition of an aqueous solution of NH₄Cl and extracted with ethyl acetate. Organic layers were combined, dried over Na₂SO₄ and concentrated. The crude product was purified by silica gel (methanol:DCM=1:20) to give N-benzylpentan-3-amine (2.47 g, 60%). LC-MS: m/z 178.3 [M+H]⁺

### N-benzyl-2-bromo-N-(pentan-3-yl)acetamide (2)

To a mixture of compound 1 (1 g, 5.6 mmol) and 2-bromoacetyl bromide (1.36 g, 6.72 mmol) in DCM (10 mL) was added NEt₃ (1.7 g, 16.8 mmol). The resulting mixture was stirred at room temperature for 3 h. The reaction was quenched by an aqueous solution of NH₄Cl and extracted with ethyl acetate. Organic layers were combined, dried over Na₂SO₄, concentrated and purified by silica gel (petroleum ether:ethyl acetate=1:1) to give N-benzyl-2-bromo-N-(pentan-3-yl)acetamide (1.28 g, 82%). LC-MS: m/z 298.2/300.2 [M+H]⁺

### 2-(5-amino-2′,4′-dioxo-2,3-dihydrospiro[indene-1,5′-ozazolidine]-3′-yl)-N-benzyl-N-(pentan-3-yl)acetamide (3)

To a mixture of compound 2 (54 mg, 0.12 mmol) and intermediate 39 (27 mg, 0.12 mmol) in DMF (5 mL) was added K₂CO₃ (50 mg, 0.36 mmol). The resulting mixture was stirred at rt for 1 h. The reaction was quenched by an aqueous solution of NH₄Cl and extracted with ethyl acetate. Organic layers were combined, dried over Na₂SO₄, concentrated and purified by silica gel (methanol:DCM=1:20) to give 2-(5-amino-2′,4′-dioxo-2,3-dihydrospiro[indene-1,5′-ozazolidine]-3′-yl)-N-benzyl-N-(pentan-3-yl)acetamide (50 mg, 63%). LC-MS: m/z 436.2 [M+H]⁺

### N-benzyl-2-(5-(3-methylureido)-2′,4′-dioxo-2,3-dihydrospiro[indene-1,5′-ozazolidine]-3′-yl)-N-(pentan-3-yl)acetamide

To a mixture of compound 3 (25 mg, 0.057 mmol) and MeNH₂ (18 mg, 0.57 mmol) in DCM (3 mL) was added NEt₃ (17 mg, 0.17 mmol). The resulting mixture was stirred at rt for 30 min. Then triphosgene (8.5 mg, 0.028 mmol) was added and stirring was continued for another 30 min. The reaction was quenched by an aqueous solution of NH₄Cl and extracted with ethyl acetate. Organic layers were combined, dried over Na₂SO₄, concentrated and purified by silica gel (methanol:DCM=1:20) to give compound 194-1 (11 mg, 63%) as a white solid. LC-MS: m/z=493.2 [M+H]⁺ at RT 4.86 (98.69% purity). ¹H NMR (400 MHz, CD₃OD-d₄) δ 7.56 (s, 1H), 7.54-7.21 (m, 7H), 4.79-4.67 (m, 4H), 3.76 (m, 1H), 3.33-3.00 (m, 2H), 2.80-2.60 (m, 3H), 2.60-2.50 (m, 2H), 1.60 (m, 4H), 0.90 (m, 6H).

### 2-amino-N-(3′-(2-(benzyl(pentan-3-yl)amino)-2-oxoethyl)-2′,4′-dioxo-2,3-dihydrospiro[indene-1,5′-ozazolidine]-5-yl)-2-methylpropanamide

To a mixture of compound 3 (25 mg, 0.057 mmol) and 2-amino-2-methylpropanoyl chloride (14 mg, 0.11 mmol) in DCM (3 mL) was added pyridine (22 mg, 0.28 mmol). The resulting mixture was stirred at rt for 1 h. The reaction was quenched by an aqueous solution of NH₄Cl and extracted with ethyl acetate. Organic layers were combined, dried over Na₂SO₄, concentrated and purified by silica gel (methanol:DCM=1:20) to give compound 194-2 (16 mg, 60%) as a white solid. LC-MS: m/z=521.3 [M+H]⁺ at RT 4.32 (97.80% purity). ¹H NMR (400 MHz, CD₃OD-d₄) δ 7.77 (s, 1H), 7.49-7.15 (m, 7H), 4.75-4.60 (m, 4H), 3.76 (m, 1H), 3.63-3.00 (m, 3H), 2.84 (m, 1H), 2.61 (m, 1H), 1.75-1.40 (m, 10H), 0.90 (m, 6H).

### Example 195

### tert-butyl (R)-1-((S)-1-(2-(benzyl((S)-1-cyclopropylethyl)amino)-2-oxoethyl)-2,5-dioxo-2′,3′-dihydrospiro[imidazolidine-4,1′-indene]-5′-ylamino)-1-oxopropan-2-ylcarbamate (1)

To a stirring solution of (R)-2-(tert-butoxycarbonylamino)propanoic acid (48 mg, 0.25 mmol) and 4-methylmorpholine (26 mg, 0.25 mmol) in dry THF (5 mL) at −15° C. was added slowly isobutyl carbonochloridate (34 mg, 0.25 mmol) and stirred for 15 min. Then intermediate 33 (110 mg, 0.25 mmol) was added. The resulting reaction mixture was warmed to RT and stirred overnight. After consumption of the starting material (by TLC), the reaction mixture was concentrated under reduced pressure to give crude compound 1 (0.15 g, 100%) which was used for the next step without further purification. LC-MS: m/z=604.3 [M+H]⁺

### (R)-2-amino-N—((S)-1-(2-(benzyl((S)-1-cyclopropylethyl)amino)-2-oxoethyl)-2,5-dioxo-2′,3′-dihydrospiro[imidazolidine-4,1′-indene]-5′-yl)propanamide

To a stirring solution of compound 1 (150 mg, 0.25 mmol) in DCM (5 mL) was added TFA (0.5 mL). The reaction mixture was stirred at room temperature for 2 h. After consumption of the starting material (by TLC), the reaction mixture was concentrated under reduced pressure and the crude product was purified by Prep-HPLC, eluting with a gradient of CH₃CN/H₂O (containing 0.1% TFA) from 10/90 to 70/30 to give the desired product compound 195-1 (49 mg, 39%) as TFA salt. LC-MS: m/z=504.2 [M+H]⁺ at RT 3.56 (97.29% purity). ¹H NMR (400 MHz, DMSO-d₆) δ 7.66 (s, 1H), 7.49-7.15 (m, 7H), 4.79-4.67 (m, 2H), 4.49-4.27 (m, 2H), 3.83-3.77 (m, 0.5H), 3.63-3.47 (m, 1.5H), 3.17-3.05 (m, 2H), 2.77-2.64 (m, 1H), 2.38-2.29 (m, 1H), 1.43-1.12 (m, 6H), 0.99-0.87 (m, 1H), 0.61-0.22 (m, 4H).

### Example 196

### 2-(5′-(1H-imidazol-2-ylamino)-2,5-dioxo-2′,3′-dihydrospiro[imidazolidine-4,1′-indene]-1-yl)-N-benzyl-N—((S)-1-cyclopropylethyl)acetamide

To a mixture of intermediate 18 (25 mg, 0.057 mmol) and 2-bromoimidazole (14 mg, 0.11 mmol) in propan-2-ol (3 mL) was added 4-methylbenzenesulfonic acid (20 mg, 0.12 mmol). The resulting mixture was microwaved at 125° C. for 1 h. The mixture was diluted with an aqueous solution of NaHCO₃ and extracted with ethyl acetate. Organic layers were combined, dried over Na₂SO₄, concentrated and purified by Prep-HPLC, eluting with a gradient of CH₃CN/H₂O (containing 0.1% TFA) from 10/90 to 70/30 to give the desired product compound 196-1 (10 mg, 7%) as a white solid. LC-MS: m/z=499.2 [M+H]⁺ at RT 3.85 (96.20% purity). ¹H NMR (400 MHz, DMSO-d₆) δ 12.93 (s, 1H), 10.30 (s, 1H), 8.82 (m, 1H), 7.50-7.00 (m, 10H), 4.79-4.10 (m, 5H), 3.75 (m, 1H), 3.04 (m, 2H), 2.63-2.10 (m, 2H), 1.19-0.87 (m, 4H), 0.51-0.02 (m, 4H).

### Example 197

### tert-butyl 1-((S)-1-(2-(benzyl((S)-1-cyclopropylethyl)amino)-2-oxoethyl)-2,5-dioxo-2′,3′-dihydrospiro[imidazolidine-4,1′-indene]-5′-ylcarbamoyl)cyclopropylcarbamate (1)

To a mixture of intermediate 33 (2.5 g, 23 mmol) and SM 1 (70 mg, 0.16 mmol) in THF (5 mL) were added NEt₃ (49 mg, 0.48 mmol) and T₃P (102 mg, 0.32 mmol). The resulting mixture was stirred at rt for 2 h. The reaction was quenched with an aqueous solution of NH₄Cl and extracted with ethyl acetate. Organic layers were combined, dried over Na₂SO₄, concentrated and purified by silica gel (methanol:DCM=1:20) to give tert-butyl 1-((S)-1-(2-(benzyl((S)-1-cyclopropylethyl)amino)-2-oxoethyl)-2,5-dioxo-2′,3′-dihydrospiro[imidazolidine-4,1′-indene]-5′-ylcarbamoyl)cyclopropylcarbamate (70 mg, 70%). LC-MS: m/z 516.3 [M+H-Boc]⁺

### 1-amino-N—((S)-1-(2-(benzyl((S)-1-cyclopropylethyl)amino)-2-oxoethyl)-2,5-dioxo-2′,3′-dihydrospiro[imidazolidine-4,1′-indene]-5′-yl)cyclopropanecarboxamide

To a solution of compound 1 (70 mg, 0.11 mmol) in DCM (5 mL) was added TFA (1 mL). The resulting solution was stirred at rt for 30 min. The reaction was quenched with an aqueous solution of NaHCO₃ and extracted with ethyl acetate. Organic layers were combined, dried over Na₂SO₄, concentrated and purified by Prep-HPLC, eluting with a gradient of CH₃CN/H₂O (containing 0.1% TFA) from 10/90 to 70/30 to give the desired product compound 197-1 (44 mg, 64%). LC-MS: m/z=516.2 [M+H]⁺ at RT 3.98 (98.90% purity). ¹H NMR (400 MHz, DMSO-d₆) δ 9.32 (s, 1H), 8.76 (s, 1H), 8.55 (s, 2H), 7.49-7.05 (m, 7H), 4.79-4.62 (m, 2H), 4.49-4.17 (m, 2H), 3.71 (m, 1H), 3.03 (m, 2H), 2.77-2.20 (m, 2H), 1.77-0.80 (m, 8H), 0.61-0.05 (m, 4H).

### Example 198

### 2-(5-(azetidin-3-ylmethylamino)-2′,4′-dioxo-2,3-dihydrospiro[indene-1,5′-ozazolidine]-3′-yl)-N-benzyl-N—((S)-1-cyclopropylethyl)acetamide

To a solution of intermediate 28 (50 mg, 0.1 mmol) and azetidin-3-ylmethanamine SM 1 (86 mg, 1 mmol) in DMSO (5 mL) were added K₃PO₄ (127 mg, 0.6 mmol), CuI (21 mg, 0.11 mmol) and L-proline (13 mg, 0.11 mmol). The resulting mixture was stirred at 90° C. for 5 h under N₂. After cooling to RT, the mixture was diluted with water, extracted with EtOAc, concentrated under reduced pressure to give a crude product which was purified by Prep-HPLC, eluting with a gradient of CH₃CN/H₂O (containing 0.1% TFA) from 10/90 to 70/30 to give the desired product compound 198-1 (20 mg, 40%) as a white solid. LC-MS: m/z=503.2 [M+H]⁺ at RT 4.01 (99.27% purity). ¹H NMR (301 MHz, CD₃OD) δ 7.57-7.05 (m, 5H), 6.52-6.27 (m, 1H), 4.83-4.71 (m, 2H), 4.70-4.46 (m, 2H), 4.45-4.39 (m, 1H), 4.11-3.98 (m, 2H), 3.89-3.77 (m, 1H), 3.75-3.61 (m, 2H), 3.23-2.91 (m, 4H), 2.82-2.65 (m, 1H), 2.60-2.43 (m, 1H), 1.38-1.18 (m, 3H), 1.04-0.89 (m, 1H), 0.69-0.24 (m, 4H).

### Example 199

### N-benzyl-N—((R)-1-cyclopropyl-2,2,2-trifluoroethyl)-2-((S)-2,5-dioxo-2′,3′-dihydrospiro[imidazolidine-4,1′-indene]-1-yl)acetamide

To a solution of intermediate 9 (52 mg, 0.2 mmol) and intermediate 41 (46 mg, 0.2 mmol) in pyridine (5 mL) was added POCl₃ (91 mg, 0.6 mmol) at RT under N₂. The mixture was stirred at RT for 1 h, quenched with water, extracted with EtOAc and concentrated under reduced pressure to give a crude product which was purified by Prep-HPLC, eluting with a gradient of CH₃CN/H₂O (containing 0.1% TFA) from 10/90 to 70/30 to give the desired product compound 199-1 (20 mg, 22%) as a yellow solid. LC-MS: m/z=472.2 [M+H]⁺ at RT 4.95 (94.41% purity). ¹H NMR (300 MHz, CD₃OD) δ 7.75-6.88 (m, 5H), 5.19-4.88 (m, 2H), 4.62-4.17 (m, 3H), 3.22-2.98 (m, 2H), 2.80-2.61 (m, 1H), 2.42-2.21 (m, 1H), 1.24-1.01 (m, 1H), 0.93-0.55 (m, 2H), 0.46-0.08 (m, 2H).

### Example 200

### Oxetan-3-ylmethyl methane sulfonate (1)

To a solution of oxetan-3-ylmethanol (88 mg, 1 mmol) and TEA (202 mg, 2 mmol) in DCM (5 mL) was added MsCl (114 mg, 1 mmol). The resulting mixture was then stirred at RT for 1 h under N₂, followed by concentration under reduced pressure to give crude compound 1 (220 mg, 100%). LC-MS: m/z=167.0 [M+H]⁺

### N-benzyl-N—((S)-1-cyclopropylethyl)-2-(5′-(oxetan-3-ylmethylamino)-2,5-dioxo-2′,3′-dihydrospiro[imidazolidine-4,1′-indene]-1-yl)acetamide

To a solution of intermediate 37 (43 mg, 0.1 mmol) and compound 1 (220 mg, 1 mmol) in DMF (5 mL) were added K₂CO₃ (127 mg, 0.6 mmol) and KI (17 mg, 0.1 mmol), The resulting mixture was then stirred at 80° C. for 5 h under N₂. After cooling to RT, the reaction mixture was diluted with water, extracted with EtOAc and concentrated under reduced pressure to give a crude product which was purified by Prep-HPLC, eluting with a gradient of CH₃CN/H₂O (containing 0.1% TFA) from 10/90 to 70/30 to give the desired product compound 200-1 (5 mg, 10%) as a white solid. LC-MS: m/z=503.2 [M+H]⁺ at RT 4.06 (92.07% purity). ¹H NMR (301 MHz, CD₃OD) δ 7.64-7.11 (m, 6H), 6.99-6.68 (m, 2H), 4.84-4.68 (m, 2H), 4.58-4.24 (m, 3H), 3.88-3.78 (m, 1H), 3.76-3.66 (m, 1H), 3.66-3.43 (m, 2H), 3.41-3.32 (m, 2H), 3.24-2.94 (m, 3H), 2.83-2.61 (m, 1H), 2.43-2.25 (m, 1H), 1.40-1.16 (m, 3H), 1.05-0.85 (m, 1H), 0.66-0.48 (m, 1H), 0.42-0.19 (m, 3H).

### Example 201

### 5-(4,4,5,5-tetramethyl-1,3,2-dioxaborolan-2-yl)-2,3-dihydrospiro[indene-1,5′-ozazolidine]-2′,4′-dione (1)

To a mixture of intermediate 25 (1 g, 3.55 mmol) and 4,4,4′,4′,5,5,5′,5′-octamethyl-2,2′-bi(1,3,2-dioxaborolane) (1.8 g, 7.1 mmol) in dioxane (20 mL) were added Pd(dppf)Cl₂ (260 mg, 0.36 mmol) and AcOK (1 g, 10.6 mmol). The resulting mixture was stirred at 100° C. for 3 h. The mixture was then diluted with an aqueous solution of NH₄Cl and extracted with ethyl acetate. Organic layers were combined, dried over Na₂SO₄, concentrated and purified by silica gel (petroleum ether:ethyl acetate=1:1) to give 5-(4,4,5,5-tetramethyl-1,3,2-dioxaborolan-2-yl)-2,3-dihydrospiro[indene-1,5′-ozazolidine]-2′,4′-dione (800 mg, 69%) as a white solid. LC-MS: m/z 330.2 [M+H]⁺

### N-benzyl-N—((S)-1-cyclopropylethyl)-2-(2′,4′-dioxo-5-(4,4,5,5-tetramethyl-1,3,2-dioxaborolan-2-yl)-2,3-dihydrospiro[indene-1,5′-ozazolidine]-3′-yl)acetamide (2)

To a mixture of compound 1 (350 mg, 1.1 mmol) and intermediate 23 (318 mg, 1.1 mmol) in MeCN(10 mL) was added K₂CO₃ (455 mg, 3.3 mmol). The resulting mixture was stirred at rt for 1 h. The reaction was quenched with an aqueous solution of NH₄Cl and extracted with ethyl acetate. Organic layers were combined, dried over Na₂SO₄, concentrated and purified by silica gel (methanol:DCM=1:20) to give N-benzyl-N—((S)-1-cyclopropylethyl)-2-(2′,4′-dioxo-5-(4,4,5,5-tetramethyl-1,3,2-dioxaborolan-2-yl)-2,3-dihydrospiro[indene-1,5′-ozazolidine]-3′-yl)acetamide (450 mg, 75%). LC-MS: m/z=545.4 [M+H]⁺

### N-benzyl-N—((S)-1-cyclopropylethyl)-2-(5-hydroxy-2′,4′-dioxo-2,3-dihydrospiro[indene-1,5′-ozazolidine]-3′-yl)acetamide (3)

To a solution of compound 2 (450 mg, 0.83 mmol) in MeOH (5 mL) was added H₂O₂ (3 mL). The resulting mixture was stirred at rt for 4 h. The reaction was quenched with an aqueous solution of NH₄Cl and extracted with ethyl acetate. Organic layers were combined, dried over Na₂SO₄, concentrated and purified by silica gel (methanol:DCM=1:20) to give N-benzyl-N—((S)-1-cyclopropylethyl)-2-(5-hydroxy-2′,4′-dioxo-2,3-dihydrospiro[indene-1,5′-ozazolidine]-3′-yl)acetamide (310 mg, 86%). LC-MS: m/z=435.2 [M+H]⁺

### N-benzyl-N—((S)-1-cyclopropylethyl)-2-(2′,4′-dioxo-5-(2-phenyl-1,3-dioxan-5-yloxy)-2,3-dihydrospiro[indene-1,5′-ozazolidine]-3′-yl)acetamide (4)

To a mixture of compound 3 (100 mg, 0.23 mmol) and SM 1 (62 mg, 0.34 mmol) in THF (5 mL) were added PPh₃ (90 mg, 0.35 mmol) and DIAD (56 mg, 0.28 mmol). The resulting mixture was stirred at rt overnight. The reaction was quenched with an aqueous solution of NH₄Cl and extracted with ethyl acetate. Organic layers were combined, dried over Na₂SO₄, concentrated and purified by silica gel (methanol:DCM=1:20) to give N-benzyl-N—((S)-1-cyclopropylethyl)-2-(2′,4′-dioxo-5-(2-phenyl-1,3-dioxan-5-yloxy)-2,3-dihydrospiro[indene-1,5′-ozazolidine]-3′-yl)acetamide (50 mg, 36%) as a white solid. LC-MS: m/z=597.6 [M+H]⁺

### N-benzyl-N—((S)-1-cyclopropylethyl)-2-(5-(1,3-dihydroxypropan-2-yloxy)-2′,4′-dioxo-2,3-dihydrospiro[indene-1,5′-ozazolidine]-3′-yl)acetamide

To a solution of compound 4 (50 mg, 0.08 mmol) in MeOH (5 mL) at room temperature was bubbled with HCl gas for 15 min. The resulting mixture was stirred for additional one hour. The mixture was then concentrated and purified by Prep-HPLC, eluting with a gradient of CH₃CN/H₂O (containing 0.1% TFA) from 10/90 to 70/30 to give the desired product compound 201-1 (3 mg, 7%) as a white solid. LC-MS: m/z=509.2 [M+H]⁺ at RT 4.26 (98.46% purity). ¹H NMR (400 MHz, CD₃OD-d₄) δ 7.49-7.15 (m, 6H), 7.10-6.85 (m, 2H), 4.79-4.30 (m, 6H), 3.85-3.40 (m, 4H), 3.30-3.00 (m, 3H), 2.75-2.49 (m, 3H), 1.43-1.20 (m, 3H), 0.99-0.22 (m, 7H).

### Example 202

### 1-amino-N-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)cyclobutanecarboxamide

To a solution of intermediate 40 (100 mg, 0.22 mmol) and SM 1 (75 mg, 7.1 mmol) in DCM (5 mL) was added pyridine (1 mL). The resulting mixture was stirred at rt overnight. It was then quenched with an aqueous solution of NH₄Cl and extracted with ethyl acetate. Organic layers were combined, dried over Na₂SO₄, concentrated and purified by Prep-HPLC, eluting with a gradient of CH₃CN/H₂O (containing 0.1% TFA) from 10/90 to 70/30 to give the desired product compound 202-1 as a TFA salt. LC-MS: m/z=549.2 [M+H]⁺ at RT 4.06 (99.54% purity). ¹H NMR (400 MHz, CD₃OD-d₃) δ 7.78 (s, 1H), 7.57-7.50 (m, 2H), 7.45-6.95 (m, 3H), 4.79-4.30 (m, 4H), 4.49-4.27 (m, 2H), 3.85 (m, 0.5H), 3.50-3.00 (m, 2.5H), 3.00-2.25 (m, 8H), 1.33-1.25 (m, 3H), 1.10 (m, 1H), 0.75-0.25 (m, 4H)

### Example 203

### tert-butyl 3-(3′-(2-(benzyl((S)-1-cyclopropylethyl)amino)-2-oxoethyl)-2′,4′-dioxo-2,3-dihydrospiro[indene-1,5′-ozazolidine]-5-ylcarbamoyl)azetidine-1-carboxylate (1)

To a stirring solution of Intermediate 48 (401 mg, 1 mmol) and intermediate 23 (296 mg, 1 mmol) in DMF (3 mL) was added K₂CO₃ (276 mg, 2 mmol) and the resulting mixture was stirred at room temperature for 1 h. After consumption of the starting material (by TLC), the reaction mixture was diluted with water and extracted with EtOAc. Combined organic extract was dried over anhydrous Na₂SO₄ and concentrated under reduced pressure to give crude compound 1 (530 mg, 86%) which was used in the next step directly. LC-MS: m/z=517.2 [M+H-Boc]⁺

### N-(3′-(2-(benzyl((S)-1-cyclopropylethyl)amino)-2-oxoethyl)-2′,4′-dioxo-2,3-dihydrospiro[indene-1,5′-ozazolidine]-5-yl)azetidine-3-carboxamide

To a stirring solution of compound 1 (530 mg, 0.86 mmol) in DCM (15 mL) was added TFA (3 mL). The reaction mixture was stirred at room temperature for 1 h. After consumption of the starting material (by TLC), the reaction mixture was concentrated under reduced pressure to give a crude product which was purified by Prep-HPLC, eluting with a gradient of CH₃CN/H₂O (containing 0.1% TFA) from 10/90 to 70/30 to give the desired product compound 203-1 (305 mg, 56%) as TFA salt. LC-MS: m/z=517.2 [M+H]⁺ at RT 4.10 (97.84% purity). ¹H NMR (301 MHz, DMSO-d₆) δ 10.35 (s, 1H), 8.77 (s, 1H), 7.77 (s, 1H), 7.55-7.10 (m, 7H), 4.80 (s, 1H), 4.72-4.58 (m, 1H), 4.58-4.41 (m, 1H), 4.39-4.28 (m, 1H), 4.10 (s, 4H), 3.83-3.74 (m, 1.5H), 3.41-3.35 (m, 0.5H), 3.24-2.99 (m, 2H), 2.77-2.53 (m, 2H), 1.35-1.07 (m, 3H), 1.05-0.93 (m, 1H), 0.61-0.09 (m, 4H).

### Example 204

### N-benzyl-2-(5-cyano-2′,4′-dioxo-2,3-dihydrospiro[indene-1,5′-ozazolidine]-3′-yl)-N—((S)-1-cyclopropylethyl)acetamide (1)

To a stirring solution of intermediate 28 (480 mg, 0.97 mmol) in DMF (2 mL) was added Zn(CN)₂ (284 mg, 2.42 mmol), followed by Pd(PPh₃)₄ (34 mg, 0.03 mmol). The resulting mixture was heated to 165° C. for 30 min by microwave. The reaction mixture was concentrated under reduced pressure and purified by silica gel column chromatography eluting with 50% EtOAc in PE to afford compound 1 (363 mg, 85%) as a yellow solid. LC-MS: m/z=444.2 [M+H]⁺

### N-benzyl-N—((S)-1-cyclopropylethyl)-2-(5-formyl-2′,4′-dioxo-2,3-dihydrospiro[indene-1,5′-ozazolidine]-3′-yl)acetamide (2)

To a stirring suspension of formic acid (4 mL) and Raney-Ni (40 mg) was added compound 1 (363 mg, 0.82 mmol). The resulting mixture was heated to 100° C. for 30 min. After consumption of the starting material (by TLC), the reaction mixture was filtered. The filtrate was concentrated under reduced pressure to give a crude product which was purified by silica gel column chromatography eluting with 50% EtOAc in PE to afford compound 2 (166 mg, 44%) as an off-white solid. LC-MS: m/z=447.2 [M+H]⁺

### 4-((3′-(2-(benzyl((S)-1-cyclopropylethyl)amino)-2-oxoethyl)-2′,4′-dioxo-2,3-dihydrospiro[indene-1,5′-ozazolidine]-5-yl)methylamino)benzoic acid

To a stirring solution of compound 2 (80 mg, 0.18 mmol) and 4-aminobenzoic acid (27 mg, 0.2 mmol) in DCM (6 mL) under N₂ was added TiCl(OiPr)₃ (375 mg, 1.44 mmol). The solution was stirred at room temperature for 2 h before addition of NaBH(OAc)₃ (305 mg, 1.44 mmol) and CH₃COOH(8 mg). stirring was continued overnight. The reaction mixture was concentrated under reduced pressure and the residue was purified by Prep-HPLC, eluting with a gradient of CH₃CN/H₂O (containing 0.1% TFA) from 10/90 to 70/30 to give the desired product compound 204-1 (32 mg, 31%) as a light yellow solid. LC-MS: m/z=568.2 [M+H]⁺ at RT 5.22 (96.91% purity). ¹H NMR (301 MHz, DMSO-d₆) δ 12.01 (s, 1H), 7.64 (d, J=8.7 Hz, 2H), 7.48-7.36 (m, 4H), 7.35-6.95 (m, 5H), 6.64-6.50 (m, 2H), 4.78 (s, 1H), 4.70-4.58 (m, 1H), 4.55-4.39 (m, 4H), 3.79-3.72 (m, 0.5H), 3.38-3.34 (m, 0.5H), 3.23-2.96 (m, 2H), 2.73-2.62 (m, 2H), 1.16 (dd, J=20.9, 6.6 Hz, 3H), 1.03-0.95 (m, 1H), 0.51-0.15 (m, 4H).

### Example 205

### (2,2-dimethyl-1,3-dioxolan-4-yl)methanol (1)

To a solution of SM 1 (3 g, 32.6 mmol) and SM 2 (3.4 g, 32.6 mmol) in acetone (30 mL) was added TsOH (560 mg, 3.26 mmol). The resulting mixture was stirred at rt for 16 h. The reaction was quenched with an aqueous solution of NH₄Cl and extracted with ethyl acetate. Organic layers were combined, dried over Na₂SO₄, concentrated and purified by silica gel (methanol:DCM=1:20) to give (2,2-dimethyl-1,3-dioxolan-4-yl)methanol (3.5 g, 81%). LC-MS: m/z=133.2 [M+H]⁺

### 2,2-dimethyl-1,3-dioxolane-4-carboxylic acid (2)

To a mixture of compound 1 (350 mg, 1.1 mmol) and RuCl₃ (318 mg, 1.1 mmol) in MeCN (10 mL) and water (5 mL) were added K₂CO₃ (455 mg, 3.3 mmol), TCCA and TBAB. The resulting mixture was stirred at rt for 3 h. The reaction was quenched with an aqueous solution of NH₄Cl and extracted with ethyl acetate. Organic layers were combined, dried over Na₂SO₄, concentrated to give 2,2-dimethyl-1,3-dioxolane-4-carboxylic acid (450 mg, 75%). LC-MS: m/z=147.4 [M+H]⁺

### 2,2-dimethyl-1,3-dioxolane-4-carbonyl chloride (3)

To a solution of compound 2 (100 mg, 0.68 mmol) in DCM (5 mL) was added oxalyl dichloride (432 mg, 3.4 mmol). The resulting mixture was stirred at rt for 16 h. The mixture was concentrated to give the crude product, 2,2-dimethyl-1,3-dioxolane-4-carbonyl chloride (100 mg, 89%).

### N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2,2-dimethyl-1,3-dioxolane-4-carboxamide (4)

To a mixture of compound 3 (100 mg, 0.18 mmol) and intermediate 18 (77 mg, 0.18 mmol) in THF (3 mL) was added pyridine (42 mg, 0.54 mmol). The resulting mixture was stirred at rt for 1 h. The reaction was quenched with an aqueous solution of NH₄Cl and extracted with ethyl acetate. Organic layers were combined, dried over Na₂SO₄, concentrated and purified by silica gel (methanol:DCM=1:20) to give N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2,2-dimethyl-1,3-dioxolane-4-carboxamide (52 mg, 51%) as a white solid. LC-MS: m/z=562.6 [M+H]⁺

### N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2,3-dihydroxypropanamide

To a solution of compound 4 (52 mg, 0.09 mmol) in DCM (5 mL) was added TFA (2 mL). The resulting solution was stirred at rt for 1 h. The mixture was concentrated and purified by Prep-HPLC, eluting with a gradient of CH₃CN/H₂O (containing 0.1% TFA) from 10/90 to 70/30 to give the desired product compound 205-1 (10 mg, 21%) as a white solid. LC-MS: m/z=522.0 [M+H]⁺ at RT 4.40 (100% purity). ¹H NMR (400 MHz, DMSO-d₆) δ 7.80 (s, 1H), 7.53-7.29 (m, 7H), 4.88-4.40 (m, 4H), 4.22 (s, 1H), 3.85 (s, 3H), 3.33-3.15 (m, 2H), 2.75 (m, 1H), 2.60 (m, 1H), 1.31-1.24 (m, 6H), 0.98 (m, 1H), 0.61-0.22 (m, 4H).

### Example 206

### N-benzyltetrahydrofuran-3-amine (1)

To a stirring solution of phenylmethanamine SM 1 (1.07 g, 10 mmol) in CH₃OH (30 mL) was added dihydrofuran-3 (2H)-one (946 mg, 11 mmol) followed by NaBH(OAc)₃ (3.18 g, 15 mmol) at 0° C. The reaction mixture was stirred at room temperature for 16 h. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 5% CH₃OH/CH₂Cl₂ to afford compound 1 (1.1 g, 62%) as a yellow oil. LC-MS: m/z 10=178.2 [M+H]⁺

### N-benzyl-2-bromo-N-(tetrahydrofuran-3-yl)acetamide (2)

To a stirring solution of compound 1 (1.1 g, 6.2 mmol) in DCM (30 mL) was added 2-bromoacetyl bromide (1.37 g, 6.82 mmol) at 0° C. The reaction mixture was stirred at room temperature for 2 h. After consumption of the starting material (by TLC), the reaction mixture was quenched by saturated NaHCO_{3aq} at 0° C. and extracted with DCM. Combined organic extracts were dried over anhydrous Na₂SO₄ and concentrated under reduced pressure to obtain crude product, which was purified by silica gel column chromatography eluting with 50% EtOAc/PE to afford compound 2 (610 mg, 33%) as a colorless oil. LC-MS: m/z=298.1, 300.1 [M+H]⁺

### 2-(5-acetamido-2′,4′-dioxo-2,3-dihydrospiro[indene-1,5′-ozazolidine]-3′-yl)-N-benzyl-N-(tetrahydrofuran-3-yl)acetamide (3)

To a stirring solution of compound 2 (69 mg, 0.23 mmol) and Intermediate 8 (55 mg, 0.21 mmol) in DMF (3 mL) was added K₂CO₃ (58 mg, 0.42 mmol) and stirred at room temperature for 1 h. After consumption of the starting material (by TLC), the reaction mixture was diluted with water and extracted with EtOAc. Combined organic extracts were dried over anhydrous Na₂SO₄ and concentrated under reduced pressure to obtain crude compound 3 (100 mg, 100%) which was used for next step directly. LC-MS: m/z=478.1 [M+H]⁺

### 2-(5-amino-2′,4′-dioxo-2,3-dihydrospiro[indene-1,5′-ozazolidine]-3′-yl)-N-benzyl-N-(tetrahydrofuran-3-yl)acetamide (4)

To a stirring solution of compound 3 (100 mg, 0.21 mmol) in CH₃OH (20 mL) was added hydrochloric acid (1 mL). The reaction mixture was heated to reflux for 2 h. After consumption of the starting material (by TLC), the reaction mixture was quenched by saturated NaHCO_{3aq} to pH=8 and extracted with EtOAc. Combined organic extracts were dried over anhydrous Na₂SO₄ and concentrated under reduced pressure to obtain crude product, which was purified by silica gel column chromatography eluting with 5% CH₃OH/CH₂Cl₂ to afford compound 4 (50 mg, 55%) as a yellow solid. LC-MS: m/z=392.1 [(M+H-44)]⁺

### N-benzyl-2-(5-(3-methylureido)-2′,4′-dioxo-2,3-dihydrospiro[indene-1,5′-ozazolidine]-3′-yl)-N-(tetrahydrofuran-3-yl)acetamide

To a stirring solution of compound 4 (50 mg, 0.11 mmol) in DCM (10 mL) was added triphosgene (17 mg, 0.6 mmol) followed by Et₃N (14 mg, 0.14 mmol) at 0° C. The reaction mixture was stirred at room temperature for 1.5 h. Then methanamine (2M in THF, 0.3 mL, 0.57 mmol) was added and stirred at room temperature for 0.5 h. After consumption of the starting material (by TLC), the reaction mixture was diluted with water and DCM. Combined organic extracts were dried over anhydrous Na₂SO₄ and concentrated under reduced pressure to obtain crude product, which was purified by Prep-HPLC, eluting with a gradient of CH₃CN/H₂O (containing 0.1% TFA) from 10/90 to 70/30 to give the desired product compound 206-1 (22 mg, 39%) as a white solid. LC-MS: m/z=493.0 [M+H]⁺ at RT 3.66 (99.62% purity). ¹H NMR (400 MHz, DMSO-d₆) δ 8.72 (d, J=4.3 Hz, 1H), 7.56 (d, J=6.9 Hz, 1H), 7.42 (t, J=7.1 Hz, 1H), 7.36-7.17 (m, 6H), 6.10 (s, 1H), 5.06-4.68 (m, 3H), 4.69-4.52 (m, 1H), 4.35 (s, 1H), 3.92-3.81 (m, 1H), 3.77-3.66 (m, 1H), 3.64-3.53 (m, 2H), 3.19-3.08 (m, 1H), 3.06-2.96 (m, 1H), 2.73-2.55 (m, 5H), 2.34-2.05 (m, 1H), 1.88-1.75 (m, 1H).

### Example 207

### N-benzyl-N—((S)-1-cyclopropylethyl)-2-(2′,4′-dioxo-5-(tetrahydro-2H-thiopyran S,S-dioxide-4-ylamino)-2,3-dihydrospiro[indene-1,5′-ozazolidine]-3′-yl)acetamide

To a solution of intermediate 18 (43 mg, 0.1 mmol) and SM 1 (29.6 mg, 0.2 mmol) in DCM (5 mL) was added, TiCl(Oi-Pr)₃ (52 mg, 0.2 mmol) and NaBH(OAc)₃ (169 mg, 0.8 mmol). The resulting mixture was stirred at rt for 18 h. Quenched by NH₄Cl aq. and extracted by ethyl acetate. Organic layer was combined, dried over Na₂SO₄ and concentrated. Purified by Prep-HPLC, eluting with a gradient of CH₃CN/H₂O (containing 0.1% TFA) from 10/90 to 70/30 to give the desired product compound 207-1 (31 mg, 55%) as white solid. LC-MS: m/z=587.9 [M+H]⁺ at RT 5.11 (97.50% purity). ¹H NMR (400 MHz, DMSO-d₆) δ 7.49-7.15 (m, 6H), 6.55 (s, 2H), 6.08 (s, 1H), 4.77-4.65 (m, 2H), 4.49-4.27 (m, 2H), 3.70 (m, 2H), 3.30-2.70 (m, 6H), 2.50 (s, 1H), 2.15-1.92 (m, 4H), 1.49-0.90 (m, 4H), 0.61-0.22 (m, 4H).

### Example 208

### 2H-spiro[1λ⁶-benzothiophene-3,4′-imidazolidine]-1,1,2′,5′-tetrone (1)

To a mixture of SM 1 (100 mg, 0.55 mmol) and KCN (107 mg, 1.65 mmol) in formamide (5 mL) was added ammonium carbonate (317 mg, 3.3 mmol). The resulting mixture was stirred at 120° C. overnight. Quenched by water and extracted by ethyl acetate. Organic layer was combined, dried over Na₂SO₄ and concentrated. Purified by Prep-HPLC, eluting with a gradient of CH₃CN/H₂O (containing 0.1% TFA) from 10/90 to 60/4 to give compound 1 (75 mg, 75%) as white solid. LC-MS: m/z=253.3 [M+H]⁺

### N-benzyl-N-[(1S)-1-cyclopropylethyl]-2-{1,1,2′,5′-tetraoxo-2H-spiro[1λ⁶-benzothiophene-3,4′-imidazolidine]-1′-yl}acetamide

To a solution of compound 1 (40 mg, 0.16 mmol) in MeCN (3 mL) was added intermediate 23 (47 mg, 0.16 mmol) and DIEA (62 mg, 0.48 mmol). The reaction mixture was stirred at room temperature for 2 hours. Quenched by NH₄Cl aq. and extracted by ethyl acetate. Organic layer was combined, dried over Na₂SO₄ and concentrated. Purified by Combiflash (methanol:DCM=1:20) to give compound 208-1 (25 mg, 75%) as white solid. LC-MS: m/z=467.9.2 [M+H]⁺ at RT 4.26 (94.30% purity). ¹H NMR (400 MHz, DMSO-d₆) δ 9.30 (d, J=24, 1H), 7.93-7.79 (m, 4H), 7.49-7.25 (m, 4H), 4.79-3.75 (m, 7H), 1.14-1.12 (m, 3H), 1.10 (m, 1H), 0.50-0.10 (m, 4H).

### Example 209

### (R)-2-(tert-butoxycarbonylamino)-2-cyclopropylacetic acid (1)

To a solution of SM 1 (230 mg, 2 mmol) in THF (3 mL) and water (3 mL) was added (Boc)₂O (523 mg, 2.4 mmol) and NaOH (160 mg, 4 mmol). The resulting mixture was stirred at rt for 2 h. Quenched by NH₄Cl aq. and extracted by ethyl acetate. Organic layer was combined, dried over Na₂SO₄ and concentrated to give (R)-2-(tert-butoxycarbonylamino)-2-cyclopropylacetic acid (250 mg, 58%). LC-MS: m/z=116.2 [M+H-Boc]⁺

### tert-butyl-(1R)-1-cyclopropyl-2-(3′-(2-(((S)-1-cyclopropylethyl) (4-fluorobenzyl)amino)-2-oxoethyl)-2′,4′-dioxo-2,3-dihydrospiro[indene-,5′-ozazolidine]-5-ylamino)-2-oxoethylcarbamate (2)

To a solution of compound 1 (50 mg, 0.23 mmol) and intermediate 40 (104 mg, 0.23 mmol) in DCM (10 mL) was added NEt₃ (70 mg, 0.69 mmol) and T₃P (220 mg, 0.69 mmol). The resulting mixture was stirred at rt for 2 h. The reaction was quenched by water and extracted by ethyl acetate. Organic layer was combined, dried over Na₂SO₄ and concentrated. Purified by Combiflash (methanol:DCM=1:20) to give tert-butyl (1R)-1-cyclopropyl-2-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-ylamino)-2-oxoethylcarbamate (80 mg, 54%). LC-MS: m/z=549.4 [M+H-Boc]⁺(2R)-2-amino-2-cyclopropyl-N-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)acetamide:

To a solution of compound 2 (80 mg, 0.12 mmol) in DCM (5 mL) was added TFA (2 mL). The resulting solution was stirred at rt for 1 h. The reaction was quenched by NaHCO₃ aq. and extracted by ethyl acetate. Organic layer was combined, dried over Na₂SO₄ and concentrated. Purified by Combiflash (methanol:DCM=1:20) to give compound 209-1 (55 mg, 84%) as white solid. LC-MS: m/z=549.0 [M+H]⁺ at RT 4.08 (100% purity). ¹H NMR (400 MHz, DMSO-d₆) δ 10.49 (s, 1H), 8.36 (s, 2H), 7.74 (s, 1H), 7.49 (m, 2H), 7.26 (m, 2H), 7.12 (m, 2H), 4.78 (s, 1H), 4.65 (m, 1H), 4.49-4.27 (m, 2H), 3.75 (m, 1H), 3.50-3.00 (m, 4H), 2.77-2.54 (m, 2H), 1.25-1.02 (m, 4H), 0.98 (m, 1H), 0.80-0.10 (m, 7H).

### Example 210

### tert-butyl 3-hydroxyazetidine-1-carboxylate (1)

To a solution of SM 1 (500 mg, 6.8 mmol) in THF (10 mL) was added (Boc)₂O (1.8 g, 8.2 mmol) and DMAP (60 mg). The resulting mixture was stirred at rt for 2 h. Quenched by NH₄Cl aq. and extracted by ethyl acetate. Organic layer was combined, dried over Na₂SO₄ and concentrated. Purified by Combiflash (methanol:DCM=1:20) to give tert-butyl 3-hydroxyazetidine-1-carboxylate (850 mg, 73%). LC-MS: m/z 74.2 [M+H-Boc]⁺

### tert-butyl 3-(1-(2-(benzyl((S)-1-cyclopropylethyl)amino)-2-oxoethyl)-2,5-dioxo-2′,3′-dihydrospiro[imidazolidine-4,1′-indene]-5′-yloxy)azetidine-1-carboxylate (2)

To a solution of compound 1 (52 mg, 0.3 mmol) and intermediate 27 (50 mg, 0.1 mmol) in toluene (5 mL) was added CuI (19 mg, 0.1 mmol), 1, 10-phenanthroline (36 mg, 0.2 mmol) and Cs₂CO₃ (98 mg, 0.3 mmol). The resulting mixture was stirred at 110° C. overnight. The reaction was quenched by NH₄Cl aq. and extracted by ethyl acetate. Organic layer was combined, dried over Na₂SO₄ and concentrated. Purified by Combiflash (methanol:DCM=1:20) to give tert-butyl 3-(1-(2-(benzyl((S)-1-cyclopropylethyl)amino)-2-oxoethyl)-2,5-dioxo-2′,3′-dihydrospiro[imidazolidine-4,1′-indene]-5′-yloxy)azetidine-1-carboxylate (20 mg, 34%). LC-MS: m/z 489.4 [M+H-Boc]⁺

### 2-(5′-(azetidin-3-yloxy)-2,5-dioxo-2′,3′-dihydrospiro[imidazolidine-4,1′-indene]-1-yl)-N-benzyl-N—((S)-1-cyclopropylethyl)acetamide

To a solution of compound 2 (20 mg, 0.03 mmol) in DCM (3 mL) was added TFA (1 mL). The resulting solution was stirred at rt for 1 h. The reaction was quenched by NaHCO₃ aq. and extracted by ethyl acetate. Organic layer was combined, dried over Na₂SO₄ and concentrated. Purified by Prep-HPLC, eluting with a gradient of CH₃CN/H₂O (containing 0.1% TFA) from 10/90 to 80/20 to give the desired product compound 210-1 (5 mg, 34%) as a white solid. LC-MS: m/z=489.0 [M+H]⁺ at RT 3.56 (97.79% purity). ¹H NMR (400 MHz, CD₃OD-d₄) δ 7.49-7.15 (m, 6H), 6.81-6.77 (m, 2H), 5.15 (m, 1H), 4.88-4.75 (m, 1H), 4.70-4.25 (m, 4H), 4.20-4.00 (m, 2H), 3.83-3.75 (m, 2H), 3.17 (m, 2H), 2.77 (m, 1H), 2.37 (m, 1H), 1.33-1.22 (m, 3H), 0.98 (m, 1H), 0.61-0.22 (m, 4H).

### Example 211

### N-(cyclopropylmethyl)-N-(2-methylbenzyl)-2-(5-(3-methylureido)-2′,4′-dioxo-2,3-dihydrospiro[indene-1,5′-ozazolidine]-3′-yl)acetamide

The procedure from Example 175 was used, substituting 2-methylbenzaldehyde and cyclopropylmethanamine. LC-MS: m/z=491.0 [M+H]⁺ at RT 4.45 (97.66% purity). ¹H NMR (400 MHz, CD₃OD-d₄) δ 7.54 (s, 1H), 7.40 (m, 7H), 7.26-7.10 (m, 6H), 4.79-4.67 (m, 2H), 4.87-4.30 (m, 4H), 4.00-3.65 (m, 2H), 3.33-3.00 (m, 2H), 2.79 (s, 3H), 2.60 (m, 1H), 2.38-2.25 (m, 3H), 1.31 (s, 1H), 1.11 (m, 1H), 0.61-0.48 (m, 2H), 0.25-0.15 (m, 2H).

### Example 212

### N-ethyl-N-(2-methylbenzyl)-2-(5-(3-methylureido)-2′,4′-dioxo-2,3-dihydrospiro[indene-1,5′-ozazolidine]-3′-yl)acetamide

The procedure from Example 175 was used, substituting 2-methylbenzaldehyde and ethanamine. LC-MS: m/z=465.2 [M+H]⁺ at RT 4.37 (99.06% purity). ¹H NMR (400 MHz, DMSO-d₆) δ 8.72 (s, 1H), 7.57 (s, 1H), 7.33-7.10 (m, 7H), 6.09 (s, 1H), 4.75-4.50 (m, 3H), 4.37 (s, 1H), 3.12-2.90 (m, 3H), 2.65 (s, 3H), 2.65-2.24 (m, 3H), 1.20-1.02 (m, 3H).

### Example 213

### tert-butyl 3-oxopyrrolidine-1-carboxylate (1)

To a solution of SM 1 (1 g, 11.7 mmol) in THF (20 mL) was added (Boc)₂O (2.7 g, 12.3 mmol) and NEt₃ (3.5 g, 35 mmol). The resulting mixture was stirred at rt for 2 h. Quenched by NH₄Cl aq. and extracted by ethyl acetate. Organic layer was combined, dried over Na₂SO₄ and concentrated to give tert-butyl 3-oxopyrrolidine-1-carboxylate (231 mg, 11%). LC-MS: m/z 86.2 [M+H-Boc]⁺

### tert-butyl 3-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-ylamino)pyrrolidine-1-carboxylate (2)

To a solution of compound 1 (50 mg, 0.23 mmol) and intermediate 40 (104 mg, 0.23 mmol) in MeOH (3 mL) was added NaBH(OAc)₃ (70 mg, 0.69 mmol) and AcOH (220 mg, 0.69 mmol). The resulting mixture was stirred at rt overnight. The reaction was quenched by NaHCO₃ aq. and extracted by ethyl acetate. Organic layer was combined, dried over Na₂SO₄ and concentrated. Purified by Combiflash (methanol:DCM=1:20) to give tert-butyl 3-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-ylamino)pyrrolidine-1-carboxylate (68 mg, 99%). LC-MS: m/z 521.4 [M+H-Boc]⁺

### N—((S)-1-cyclopropylethyl)-2-(2′,4′-dioxo-5-(pyrrolidin-3-ylamino)-2,3-dihydrospiro[indene-1,5′-ozazolidine]-3′-yl)-N-(4-fluorobenzyl)acetamide

To a solution of compound 2 (68 mg, 0.11 mmol) in DCM (3 mL) was added TFA (1 mL). The resulting solution was stirred at rt for 1 h. The reaction was quenched by NaHCO₃ aq. and extracted by ethyl acetate. Organic layer was combined, dried over Na₂SO₄ and concentrated. Purified by Combiflash (methanol:DCM=1:20) to give N—((S)-1-cyclopropylethyl)-2-(2′,4′-dioxo-5-(pyrrolidin-3-ylamino)-2,3-dihydrospiro[indene-1,5′-ozazolidine]-3′-yl)-N-(4-fluorobenzyl)acetamide compound 213-1 (50 mg, 87%) as white solid. LC-MS: m/z=521.2 [M+H]⁺ at RT 4.04 (96.49% purity). ¹H NMR (400 MHz, CD₃OD-d₄) δ 7.49 (m, 1H), 7.34 (m, 2H), 7.34 (m, 2H), 7.16 (m, 1H), 7.04 (m, 1H), 6.61 (m, 2H), 4.89-4.30 (m, 4H), 4.24 (m, 1H), 3.84 (m, 1H), 3.53-2.95 (m, 6H), 2.75 (m, 1H), 2.55 (m, 1H), 2.40 (m, 1H), 2.11 (m, 1H), 1.33-1.24 (m, 3H), 0.97 (m, 1H), 0.59 (m, 1H), 0.45-0.20 (m, 4H).

### Example 214

### tert-butyl cyanomethyl carbonate (1)

To a solution of SM 1 (1.53 g, 51 mmol) in THF (50 mL) was added (Boc)₂O (23 g, 12.3 mmol) and NEt₃ (10.3 g, 35 mmol). The resulting mixture was stirred at rt for 2 h. Quenched by NH₄Cl aq. and extracted by ethyl acetate. Organic layer was combined, dried over Na₂SO₄ and concentrated to give tert-butyl cyanomethyl carbonate (3.7 mg, 99%). tert-butyl 1-(hydroxymethyl)cyclopropylcarbamate (2):

To a solution of compound 1 (1.57 mg, 10 mmol) in Et₂O (30 mL) was added EtMgBr (20 mL 1M in Et₂O, 20 mmol) and Ti(Oi-Pr)₄ (57 mg, 0.2 mmol). The resulting mixture was stirred at 0 degree for 1 h. The reaction was quenched by NH₄Cl aq. and extracted by ethyl acetate. Organic layer was combined, dried over Na₂SO₄ and concentrated. Recrystallization to give tert-butyl 1-(hydroxymethyl)cyclopropylcarbamate (457 mg, 24%). LC-MS: m/z 88.4 [M+H-Boc]⁺

### tert-butyl 1-formylcyclopropylcarbamate (3)

To a solution of oxalyl dichloride (277 mg, 2.2 mmol) in DCM (5 mL) was added DMSO (1 mL) and NEt₃ (333 mg, 3.3 mmol). The resulting solution was stirred at −78° C. for 1 h. Then a solution of compound 2 (200 mg, 1.1 mmol) in 2 mL DCM was added. The reaction was stirred −78° C. for 1 h. Quenched by NH₄Cl aq. and extracted by ethyl acetate. Organic layer was combined, dried over Na₂SO₄ and concentrated to give tert-butyl 1-formylcyclopropylcarbamate (196 mg, 99%).

The procedure from Example 213 was used for step 4 and step 5, substituting tert-butyl 1-formylcyclopropylcarbamate.

### 2-(5-((1-aminocyclopropyl)methylamino)-2′,4′-dioxo-2,3-dihydrospiro[indene-1,5′-ozazolidine]-3′-yl)-N—((S)-1-cyclopropylethyl)-N-(4-fluorobenzyl)acetamide

LC-MS: m/z=521.1 [M+H]⁺ at RT 4.07 (97.46% purity). ¹H NMR (400 MHz, CD₃OD-d₄) δ 7.49 (m, 1H), 7.34 (m, 2H), 7.34 (m, 2H), 7.16 (m, 1H), 7.04 (m, 1H), 6.67 (m, 2H), 4.80-4.25 (m, 4H), 3.84 (m, 0.5H), 3.62 (s, 0.5H), 3.33 (s, 2H), 3.25-2.98 (m, 2H), 2.75 (m, 1H), 2.55 (m, 1H), 1.33-1.24 (m, 3H), 1.00-0.80 (m, 6H), 0.59 (m, 1H), 0.45-0.20 (m, 4H).

### Example 215

The procedure from Example 175 was used, substituting dihydro-2H-pyran-4(3H)-one and phenylmethanamine.

### N-benzyl-2-(5-(3-methylureido)-2′,4′-dioxo-2,3-dihydrospiro[indene-1,5′-ozazolidine]-3′-yl)-N-(tetrahydro-2H-pyran-4-yl)acetamide

LC-MS: m/z=506.9 [M+H]⁺ at RT 3.78 (98.22% purity). ¹H NMR (400 MHz, DMSO-d₆) δ 8.71 (s, 1H), 7.57 (s, 1H), 7.49-7.15 (m, 7H), 6.10 (m, 1H), 4.79-4.40 (m, 4H), 4.38 (s, 1H), 4.15 (m, 1H), 3.83 (m, 2H), 3.43-2.90 (m, 4H), 2.70-2.60 (m, 4H), 2.60-2.45 (m, 1H), 1.75-1.45 (m, 4H).

### Example 216

### N-benzyl-N—((R)-1-cyclopropyl-2,2,2-trifluoroethyl)-2-(5-isocyanato-2′,4′-dioxo-2,3-dihydrospiro[indene-1,5′-ozazolidine]-3′-yl)acetamide (1)

To a solution of intermediate 47 (40 mg, 0.08 mmol) in THF (5 mL) was added TEA (48 mg, 0.48 mmol) and triphosgene (47 mg, 0.16 mmol) at 0° C. under N₂, then the reaction mixture was stirred at RT for 2 h. After consumption of the starting material (by TLC), the resulting mixture was used for next step.

### N-benzyl-N—((R)-1-cyclopropyl-2,2,2-trifluoroethyl)-2-(5-(3-methylureido)-2′,4′-dioxo-2,3-dihydrospiro[indene-1,5′-ozazolidine]-3′-yl)acetamide

To a solution of compound 1 (5 mL in THF) was added MeNH₂ (0.8 mL, 0.8 mmol, 1N in THF), then the reaction mixture was stirred at RT for 2 h. Quenched with water, extracted with EtOAc, dried and concentrated under reduced pressure to obtain crude product, which was purified by Prep-HPLC, eluting with a gradient of CH₃CN/H₂O (containing 0.1% TFA) from 10/90 to 80/20 to give the desired product Example 216-1 (20 mg, 46%) as a white solid. LC-MS: m/z=545.1 [M+H]⁺ at RT 4.70 (99.16% purity). ¹H NMR (301 MHz, CD₃OD) δ 7.86-7.09 (m, 8H), 5.24-4.93 (m, 2H), 4.72-4.21 (m, 3H), 3.25-2.95 (m, 2H), 2.87-2.62 (m, 4H), 2.64-2.40 (m, 1H), 1.22-1.09 (m, 1H), 0.89-0.58 (m, 2H), 0.51-0.08 (m, 2H).

### Example 217

### N-benzyl-2-(5-(3-methylureido)-2′,4′-dioxo-2,3-dihydrospiro[indene-1,5′-ozazolidine]-3′-yl)-N-(tetrahydro-2H-pyran-3-yl)acetamide

The procedure from Example 175 was used, substituting dihydro-2H-pyran-3(4H)-one and phenylmethanamine. LC-MS: m/z=507.1 [M+H]⁺ at RT 3.78 (98.86% purity). ¹H NMR (400 MHz, DMSO-d₆) δ 8.74 (d, J=6.0 Hz, 1H), 7.57 (s, 1H), 7.41 (t, J=7.5 Hz, 1H), 7.37-7.16 (m, 6H), 6.16-6.07 (m, 1H), 4.89-4.57 (m, 3H), 4.51-4.27 (m, 2H), 4.03-3.96 (m, 0.5H), 3.75-3.58 (m, 2H), 3.30-2.92 (m, 4.5H), 2.72-2.58 (m, 4H), 1.79-1.49 (m, 4H).

### Example 218

### N¹-(3′-(2-(benzyl((S)-1-cyclopropylethyl)amino)-2-oxoethyl)-2′,4′-dioxo-2,3-dihydrospiro[indene-1,5′-ozazolidine]-5-yl)oxalamide

To a solution of intermediate 18 (50 mg, 0.12 mmol) in DCM (5 mL) was added oxalyl dichloride (30 mg, 0.24 mmol) a. The resulting mixture was stirred at 0° C. for 30 min. Then the reaction mixture was bubbled by NH₃ gas for 10 min. Quenched by NH₄Cl aq. and extracted by ethyl acetate. Organic layer was combined, dried over Na₂SO₄ and concentrated. Purified by Prep-HPLC, eluting with a gradient of CH₃CN/H₂O (containing 0.1% TFA) from 10/90 to 80/20 to give the desired product compound 218-1 (20 mg, 33%) as a white solid. LC-MS: m/z=505.2 [M+H]⁺ at RT 4.76 (98.96% purity). ¹H NMR (400 MHz, DMSO-d₆) δ 10.74 (s, 1H), 8.34 (s, 1H), 8.03 (s, 1H), 7.92 (s, 1H), 7.73 (d, J=8.0 Hz, 1H), 7.49-7.37 (m, 3H), 7.32-7.19 (m, 3H), 4.80-4.65 (m, 2H), 4.58-4.33 (m, 2H), 3.83-3.78 (m, 0.5H), 3.40-3.34 (m, 0.5H), 3.22-3.05 (m, 2H), 2.72-2.65 (m, 1H), 2.62-2.56 (m, 1H), 1.24 (m, 3H), 0.97 (m, 1H), 0.30 (m, 4H).

### Example 219

### 2-(5-(azetidin-3-ylamino)-2′,4′-dioxo-2,3-dihydrospiro[indene-1,5′-ozazolidine]-3′-yl)-N—((S)-1-cyclopropylethyl)-N-(4-fluorobenzyl)acetamide

The procedure from Example 214 was used, substituting tert-butyl 3-hydroxyazetidine-1-carboxylate. LC-MS: m/z=507.0 [M+H]⁺ at RT 3.99 (94.22% purity). ¹H NMR (400 MHz, CD₃OD) δ 7.47 (d, J=5.3 Hz, 1H), 7.32 (d, J=8.7 Hz, 2H), 7.15 (t, J=8.7 Hz, 1H), 7.03 (t, J=8.8 Hz, 1H), 6.55 (d, J=9.8 Hz, 2H), 4.87-4.37 (m, 7H), 4.02-3.81 (m, 2.5H), 3.39-3.34 (m, 0.5H), 3.19-3.02 (m, 2H), 2.79-2.68 (m, 1H), 2.58-2.48 (m, 1H), 1.29 (dd, J=34.1, 6.7 Hz, 3H), 1.01-0.93 (m, 1H), 0.66-0.26 (m, 4H).

### Example 220

### N-benzyl-N—((S)-1-cyclopropylethyl)-2-(2′,4′-dioxo-5-(2-oxopyrrolidin-3-ylamino)-2,3-dihydrospiro[indene-1,5′-ozazolidine]-3′-yl)acetamide

To a solution of SM 1 (121 mg, 1.2 mmol) and intermediate 18 (50 mg, 0.12 mmol) in DCM (5 mL) was added trifluoromethanesulfonic anhydride (170 mg, 0.6 mmol) and DIPEA (46 mg, 0.36 mmol). The resulting mixture was stirred at −20° C. for 2 h. Then warmed it to rt. Stirred for another 16 h. Quenched by NH₄Cl aq. and extracted by ethyl acetate. Organic layer was combined, dried over Na₂SO₄ and concentrated. Purified by Combiflash (methanol:DCM=1:20) to give tert-butyl cyanomethyl carbonate compound 220-1 (7 mg, 11%). LC-MS: m/z=473.0 [M+H]⁺ at RT 4.42 (99.52% purity). ¹H NMR (400 MHz, CD₃OD) δ 7.52-7.38 (m, 3H), 7.37-7.19 (m, 3H), 6.78-6.60 (m, 2H), 4.87-4.75 (m, 1H), 4.70-4.37 (m, 2H), 4.25-4.16 (m, 1H), 3.89-3.81 (m, 0.5H), 3.46-3.35 (m, 1.5H), 3.27-2.94 (m, 3H), 2.80-2.46 (m, 4H), 2.02-1.90 (m, 1H), 1.29 (dd, J=29.2, 6.8 Hz, 3H), 1.02-0.88 (m, 1H), 0.63-0.22 (m, 4H).

### Example 221

### 3-(3′-(2-(benzyl((S)-1-cyclopropylethyl)amino)-2-oxoethyl)-2′,4′-dioxo-2,3-dihydrospiro[indene-1,5′-ozazolidine]-5-ylamino)-2,2-dimethyl-3-oxopropanoic acid

To a solution of SM 1 (158 mg, 1.2 mmol) and intermediate 18 (50 mg, 0.12 mmol) in THF (5 mL) was added pyridine (0.5 mL). The resulting mixture was stirred at rt for 18 h. Quenched by NH₄Cl aq. and extracted by ethyl acetate. Organic layer was combined, dried over Na₂SO₄ and concentrated. Purified by Prep-HPLC, eluting with a gradient of CH3CN/H2O (containing 0.1% TFA) from 10/90 to 70/30 to give the desired product compound 221-1 (30 mg, 50%) as white solid. LC-MS: m/z=548.0 [M+H]⁺ at RT 4.97 (99.60% purity). ¹H NMR (400 MHz, CD₃OD) δ 7.71 (s, 1H), 7.53-7.38 (m, 4H), 7.38-7.12 (m, 3H), 4.92-4.75 (m, 2H), 4.70-4.50 (m, 1H), 4.49-4.41 (m, 1H), 3.89-3.83 (m, 0.5H), 3.38-3.34 (m, 0.5H), 3.28-3.08 (m, 2H), 2.84-2.74 (m, 1H), 2.64-2.52 (m, 1H), 1.62-1.44 (m, 6H), 1.29 (dd, J=28.9, 6.7 Hz, 3H), 1.02-0.92 (m, 1H), 0.65-0.28 (m, 4H).

### Example 222

### N-benzyl-N—((S)-1-cyclopropylethyl)-2-(5-(2-hydroxyethylamino)-2′,4′-dioxo-2,3-dihydrospiro[indene-1,5′-ozazolidine]-3′-yl)acetamid

To a solution of and intermediate 18 (100 mg, 0.24 mmol) in acetic acid (1 mL) and water (1 mL) was added oxirane (0.2 mL). The resulting mixture was stirred at rt for 3 h. Quenched by NH₄Cl aq. and extracted by ethyl acetate. Organic layer was combined, dried over Na₂SO₄ and concentrated. Purified by Prep-HPLC, eluting with a gradient of CH₃CN/H₂O (containing 0.1% TFA) from 10/90 to 70/30 to give the desired product compound 222-1 (25 mg, 23%) as white solid. LC-MS: m/z=434.2 [M+H]⁺ at RT 4.52 (99.84% purity). ¹H NMR (400 MHz, CD₃OD) δ 7.43 (dt, J=11.8, 7.7 Hz, 3H), 7.38-7.13 (m, 3H), 6.92 (dd, J=16.0, 8.1 Hz, 2H), 4.86-4.76 (m, 2H), 4.72-4.54 (m, 1H), 4.53-4.39 (m, 1H), 3.91-3.82 (m, 0.5H), 3.75 (td, J=5.6, 2.8 Hz, 2H), 3.41-3.34 (m, 2.5H), 3.28-3.03 (m, 2H), 2.86-2.71 (m, 1H), 2.64-2.51 (m, 1H), 1.29 (dd, J=29.5, 6.8 Hz, 3H), 1.03-0.91 (m, 1H), 0.67-0.54 (m, 1H), 0.46-0.19 (m, 3H).

### Example 223

### (2R)-2-amino-N-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)propanamide

The procedure from Example 213 was used, substituting (R)-2-aminopropanoic acid. LC-MS: m/z=523.3 [M+H]⁺ at RT 3.94 (99.12% purity). ¹H NMR (400 MHz, DMSO-d₆) δ 10.63 (s, 1H), 8.24 (s, 2H), 7.71 (d, J=8.9 Hz, 1H), 7.57-7.40 (m, 3H), 7.37-7.20 (m, 2H), 7.12 (t, J=8.9 Hz, 1H), 4.78 (s, 1H), 4.71-4.58 (m, 1H), 4.55-4.26 (m, 2H), 4.04 (d, J=7.1 Hz, 1H), 3.80-3.73 (m, 0.5H), 3.44-3.34 (m, 0.5H), 3.25-3.02 (m, 2H), 2.76-2.62 (m, 1H), 2.62-2.52 (m, 1H), 1.46 (dd, J=6.9, 2.0 Hz, 3H), 1.27-1.12 (m, 3H), 1.03-0.91 (m, 1H), 0.56-0.15 (m, 4H).

### Example 224

### 2-(3′-(2-(benzyl((S)-1-cyclopropylethyl)amino)-2-oxoethyl)-2′,4′-dioxo-2,3-dihydrospiro[indene-1,5′-ozazolidine]-5-ylamino)-2-oxoacetic acid

To a solution of intermediate 18 (50 mg, 0.12 mmol) in DCM (5 mL) was added oxalyl dichloride (30 mg, 0.24 mmol) a. The resulting mixture was stirred at 0° C. for 30 min. Then the reaction mixture was quenched water. Stirred for another 1 h. Purified by Prep-HPLC, eluting with a gradient of CH₃CN/H₂O (containing 0.1% TFA) from 10/90 to 70/30 to give the desired product compound 224-1 (10 mg, 17%) as white solid. LC-MS: m/z=506.1 [M+H]⁺ at RT 4.32 (99.18% purity). ¹H NMR (400 MHz, CD₃OD) δ 7.87 (s, 1H), 7.68-7.50 (m, 2H), 7.49-7.19 (m, 5H), 4.85-4.72 (m, 2H), 4.66-4.38 (m, 2H), 3.93-3.80 (m, 0.5H), 3.37-3.33 (m, 0.5H), 3.30-3.07 (m, 2H), 2.85-2.72 (m, 1H), 2.67-2.52 (m, 1H), 1.28 (m, 3H), 1.04-0.91 (m, 1H), 0.66-0.54 (m, 1H), 0.46-0.22 (m, 3H).

### Example 225

### (S)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)-3-hydroxypropanamide

The procedure from Example 213 was used, substituting (S)-2-amino-3-hydroxypropanoic acid. LC-MS: m/z=521.1 [M+H]⁺ at RT 3.82 (99.48% purity). ¹H NMR (400 MHz, CD₃OD) δ 7.79 (s, 1H), 7.56-7.39 (m, 4H), 7.38-7.15 (m, 3H), 4.86-4.67 (m, 2H), 4.65-4.42 (m, 2H), 4.12-3.93 (m, 3H), 3.89-3.83 (m, 0.5H), 3.39-3.34 (m, 0.5H), 3.29-3.09 (m, 2H), 2.85-2.76 (m, 1H), 2.65-2.55 (m, 1H), 1.29 (dd, J=29.0, 6.8 Hz, 3H), 1.03-0.92 (m, 1H), 0.65-0.55 (m 1H), 0.45-0.27 (m, 3H).

### Example 229

### N—((S)-1-cyclopropylethyl)-2-(2′,4′-dioxo-5-(piperidin-3-ylamino)-2,3-dihydrospiro[indene-1,5′-ozazolidine]-3′-yl)-N-(4-fluorobenzyl)acetamide

The procedure from Example 213 was used, substituting piperidin-3-one. LC-MS: m/z=535.0 [M+H]⁺ at RT 4.07 (98.82% purity). ¹H NMR (400 MHz, CD₃OD) δ 7.48 (dd, J=8.3, 5.3 Hz, 1H), 7.31 (dd, J=13.0, 7.7 Hz, 2H), 7.15 (t, J=8.7 Hz, 1H), 7.03 (t, J=8.8 Hz, 1H), 6.72-6.56 (m, 2H), 4.85-4.39 (m, 3H), 3.87-3.82 (m, 0.5H), 3.80-3.71 (m, 1H), 3.51-3.42 (m, 0.5H), 3.22-3.12 (m, 1H), 3.09-2.93 (m, 2H), 2.89-2.71 (m, 2H), 2.58-2.44 (m, 1H), 2.19-2.08 (m, 2H), 1.90-1.79 (m, 1H), 1.67-1.58 (m, 1H), 1.39-1.21 (m, 5H), 1.02-0.91 (m, 1H), 0.65-0.28 (m, 4H).

### Example 227

### 2,3-dihydro-1H-inden-5-amine-d2 (1)

To a stirring suspension of LiAlD₄ (5.04 g, 120 mmol) and AlCl₃ (32 g, 240 mmol) in anhydrous Et₂O (300 mL) was added 6-amino-2,3-dihydro-1H-inden-1-one SM 1 (4.41 g, 30 mmol) slowly at 0° C. The reaction mixture was gradually warmed to room temperature and stirred at room temperature for 4 h. After consumption of the starting material (by TLC), the reaction mixture was quenched sequentially by addition of H₂O (37 mL), 15% KOH_{aq} (37 mL), H₂O (111 mL). Na₂SO₄ was added and the mixture was filtered. The organics were dried over anhydrous Na₂SO₄ and concentrated under reduced pressure to obtain crude compound 1 (3.9 g, 96%) as an oil used for next step directly. LC-MS: m/z=136.2 [M+H]⁺

### N-(2,3-dihydro-1H-inden-5-yl)acetamide-d2 (2)

To a stirring solution of compound 1 (3.9 g, 28.8 mmol) in DCM (50 mL) was added Et₃N (3.8 g, 37.4 mmol) followed by acetyl chloride (2.5 g, 31.7 mmol) at 0° C. The reaction mixture was stirred at 0° C. for 0.5 h. After consumption of the starting material (by TLC), the reaction mixture was quenched by H₂O and extracted with DCM. Combined organic extracts were dried over anhydrous Na₂SO₄ and concentrated under reduced pressure to obtain crude product, which was purified by silica gel column chromatography eluting with 50% EtOAc/PE to afford compound 2 (3.32 g, 65%) as a light yellowed solid. LC-MS: m/z=178.1 [M+H]⁺

### N-(1-oxo-2,3-dihydro-1H-inden-5-yl)acetamide-d2 (3)

To a stirring solution of compound 2 (3.32 g, 18.7 mmol) in CH₃COOH (15 mL) and Ac₂O (15 mL) was added CrO₃ (4.3 g, 43 mmol) dissolved in H₂O (5 mL) keeping the temperature between 0° C. and 5° C. The reaction mixture was stirred at room temperature for 2 h. After consumption of the starting material (by TLC), the reaction mixture was quenched with saturated NaOH_{aq} to pH=8. Filtered and the filtrated was extracted with EtOAc. Combined organic extracts were dried over anhydrous Na₂SO₄ and concentrated under reduced pressure to obtain crude product, which was purified by silica gel column chromatography eluting with 50% EtOAc/PE to afford compound 3 (2.4 g, 68%) as a light yellow solid. LC-MS: m/z=192.2 [M+H]⁺

### N-(1-cyano-1-(trimethylsilyloxy)-2,3-dihydro-1H-inden-5-yl)acetamide-d2 (4)

To a stirring solution of compound 3 (382 mg, 2 mmol) in Toluene (20 mL) and CH₃CN (20 mL) was added TMSCN (992 mg, 10 mmol) followed by ZnI₂ (128 mg, 0.4 mmol). The reaction mixture was heated to reflux for 2 h. After consumption of the starting material (by TLC), the reaction mixture was concentrated under reduced pressure to obtain crude product, which was purified by silica gel column chromatography eluting with 50% EtOAc/PE to afford compound 4 (290 mg, 50%) as a yellow oil. LC-MS: m/z=291.0 [M+H]⁺

### ethyl 5-acetamido-1-hydroxy-2,3-dihydro-1H-indene-1-carbimidate-d2 (5)

To a stirring solution of compound 4 (290 mg, 1 mmol) in anhydrous CH₃OH (20 mL) was inleted HCl (gas) at 0° C. The reaction mixture was stirred at 0° C. for 2 h. The reaction mixture was concentrated under reduced pressure to obtain crude compound 5 (270 mg, 100%) as a brown oil used for next step directly. LC-MS: m/z=265.1 [M+H]⁺

### N-(2′,4′-dioxo-2,3-dihydrospiro[indene-1,5′-ozazolidine]-5-yl)acetamide-d2 (6)

To a stirring solution of compound 5 (270 mg, 1 mmol) in anhydrous THF (20 mL) was added Et₃N (305 mg, 3 mmol) followed by triphosgene (297 mg, 1 mmol) at 0° C. The reaction mixture was stirred at 0° C. for 1 h. Then hydrochloric acid was added to pH=2. The resulting reaction mixture was stirred at 0° C. for 0.5 h. The reaction mixture was diluted with H₂O and EtOAc. Combined organic extracts were dried over anhydrous Na₂SO₄ and concentrated under reduced pressure to obtain crude product, which was purified by silica gel column chromatography eluting with 5% CH₃OH/CH₂Cl₂ to afford compound 6 (80 mg, 30%) as a yellow solid. LC-MS: m/z=263.2 [M+H]⁺

### 2-(5-acetamido-2′,4′-dioxo-2,3-dihydrospiro[indene-1,5′-ozazolidine]-3′-yl)-N-benzyl-N—((S)-1-cyclopropylethyl)acetamide-d2 (7)

To a stirring solution of compound 6 (80 mg, 0.3 mmol) and Intermediate 23 (100 mg, 0.33 mmol) in DMF (3 mL) was added K₂CO₃ (84 mg, 0.6 mmol) and stirred at room temperature for 1 h. After consumption of the starting material (by TLC), the reaction mixture was diluted with water and extracted with EtOAc. Combined organic extracts were dried over anhydrous Na₂SO₄ and concentrated under reduced pressure to obtain crude compound 7 (125 mg, 87%) which was used for next step directly. LC-MS: m/z=478.2 [M+H]⁺

### 2-(5-amino-2′,4′-dioxo-2,3-dihydrospiro[indene-1,5′-ozazolidine]-3′-yl)-N-benzyl-N—((S)-1-cyclopropylethyl)acetamide-d2 (8)

To a stirring solution of compound 7 (125 mg, 0.26 mmol) in CH₃OH (20 mL) was added hydrochloric acid (1 mL). The reaction mixture was heated to reflux for 2 h. After consumption of the starting material (by TLC), the reaction mixture was quenched by saturated NaHCO₃ aq. to pH=8 and extracted with EtOAc. Combined organic extracts were dried over anhydrous Na₂SO₄ and concentrated under reduced pressure to obtain crude product, which was purified by silica gel column chromatography eluting with 50% EtOAc/PE to afford compound 8 (50 mg, 44%) as a solid. LC-MS: m/z=392.1 [M+H-44]⁺

### N-benzyl-N—((S)-1-cyclopropylethyl)-2-(5-(3-methylureido)-2′,4′-dioxo-2,3-dihydrospiro[indene-1,5′-ozazolidine]-3′-yl)acetamide-d2

To a stirring solution of compound 8 (50 mg, 0.11 mmol) in DCM (10 mL) was added triphosgene (17 mg, 0.6 mmol) followed by Et₃N (14 mg, 0.14 mmol) at 0° C. The reaction mixture was stirred at room temperature for 1.5 h. Then methanamine (2M in THF, 0.3 mL, 0.57 mmol) was added and stirred at room temperature for 0.5 hour. After consumption of the starting material (by TLC), the reaction mixture was diluted with water and DCM. Combined organic extracts were dried over anhydrous Na₂SO₄ and concentrated under reduced pressure to obtain crude product, which was purified by Prep-HPLC, eluting with a gradient of CH₃CN/H₂O (containing 0.1% TFA) from 10/90 to 70/30 to give the desired product compound 227-1 (16 mg, 28%) as a white solid. LC-MS: m/z=493.1 [M+H]⁺ at RT 4.41 (99.09% purity). ¹H NMR (400 MHz, CD₃OD) δ 7.54 (dd, J=4.7, 1.9 Hz, 1H), 7.50-7.16 (m, 7H), 4.86-4.69 (m, 2H), 4.67-4.51 (m, 1H), 4.47-4.39 (m, 1H), 3.89-3.83 (m, 0.5H), 3.39-3.35 (m, 0.5H), 2.86-2.69 (m, 4H), 2.55 (t, J=13.4 Hz, 1H), 1.29 (dd, J=29.2, 6.8 Hz, 3H), 1.03-0.93 (m, 1H), 0.65-0.27 (m, 4H).

### Example 228

### N-(4-fluorobenzyl)-2-(5-(3-methylureido)-2′,4′-dioxo-2,3-dihydrospiro[indene-1,5′-ozazolidine]-3′-yl)-N—((S)-1,1,1-trifluoropropan-2-yl)acetamide (1)

To a stirring solution of intermediate 42 (8.6 g, 18 mmol) in THF (100 mL) was added Et₃N (7.5 mL, 54 mmol) followed by triphosgene (2.7 g, 9 mmol) in one portion at RT. The reaction mixture was stirred at RT for 30 min. Then methanamine (2M in THF, 45 mL, 90 mmol) was added and stirred at room temperature for 0.5 hour. After consumption of the starting material (by TLC), the reaction mixture was diluted with water and EtOAc. Combined organic extracts were dried over anhydrous Na₂SO₄ and concentrated under reduced pressure to obtain crude product, which was purified by silica gel column chromatography eluting with 50% EtOAc/PE to afford compound 228-1 (7.96 g, 83%) as a yellow solid. LC-MS: m/z=537.1 [M+H]⁺ at RT 4.74 (98.44% purity). ¹H NMR (301 MHz, DMSO-d₆) δ 8.72 (s, 1H), 7.55 (s, 1H), 7.41-7.32 (m, 2H), 7.29-7.09 (m, 4H), 6.08 (s, 1H), 5.46-5.42 (m, 0.5H), 4.99-4.88 (s, 1.5H), 4.83-4.69 (m, 1H), 4.68-4.54 (m, 1H), 4.24-4.16 (m, 1H), 3.13-2.95 (m, 2H), 2.67-2.47 (m, 5H), 1.43-1.32 (m, 3H).

### Example 229

### 2-amino-N-(3′-(2-(benzyl((R)-1-cyclopropyl-2,2,2-trifluoroethyl)amino)-2-oxoethyl)-2′,4′-dioxo-2,3-dihydrospiro[indene-1,5′-ozazolidine]-5-yl)-2-methylpropanamide

To a solution of intermediate 47 (40 mg, 0.08 mmol) in DCM (2 mL) was added pyridine (1 mL) and intermediate 36 (25 mg, 0.16 mmol), then the reaction mixture was stirred at RT for 2 h under N₂. Quenched with water and extracted with DCM, dried and concentrated under reduced pressure to obtain crude product, which was purified by Prep-HPLC, eluting with a gradient of CH₃CN/H₂O (containing 0.1% TFA) from 10/90 to 80/20 to give the desired product compound 229-1 (7 mg, 15%) as a white solid. LC-MS: m/z=573.2 [M+H]⁺ at RT 4.18 (95% purity). ¹H NMR (400 MHz, CD₃OD) δ 7.81-7.69 (m, 1H), 7.57-7.37 (m, 5H), 7.38-7.15 (m, 2H), 5.23-4.99 (m, 2H), 4.74-4.34 (m, 3H), 3.31-3.04 (m, 2H), 2.92-2.70 (m, 1H), 2.65-2.45 (m, 1H), 1.84-1.68 (m, 6H), 1.26-1.09 (m, 1H), 0.92-0.58 (m, 2H), 0.52-0.09 (m, 2H).

### Example 230

### N-isobutyl-N-(2-methylbenzyl)-2-(5-(3-methylureido)-2′,4′-dioxo-2,3-dihydrospiro[indene-1,5′-ozazolidine]-3′-yl)acetamide

The procedure from Example 175 was used, substituting 2-methylbenzaldehyde and 2-methylpropan-1-amine. LC-MS: m/z=493.0 [M+H]⁺ at RT 4.65 (96.3% purity). ¹H NMR (400 MHz, CD₃OD) δ 8.74 (s, 1H), 7.57 (s, 1H), 7.30-6.90 (m, 6H), 6.11 (t, J=8.7 Hz, 1H), 4.75-4.49 (m, 3H), 4.38 (s, 1H), 3.28-2.90 (m, 4H), 2.65 (s, 4H), 2.5 (m, 1H), 2.30-2.20 (m, 3H), 2.00 (m, 1H), 0.90 (dd, J=28.9, 6.7 Hz, 6H).

### Example 231

### 3,6-dioxabicyclo[3.1.0]hexane (1)

To a solution of 2,5-dihydrofuran SM 1 (5.0 g, 71 mmol) in DCM (100 mL) was added m-CPBA (18.9 g, 109 mmol) at 0° C., then the reaction mixture was stirred at reflux for 15 h. Quenched with water and extracted with EtOAc, dried and evaporated to give compound 1 (3.5 g, 57%).

### 4-aminotetrahydrofuran-3-ol (2)

To a solution of compound 1 (0.3 g, 3.5 mmol) in isopropanol (5 mL) and ammonium hydroxide (10 mL) was stirred at 80° C. for 15 h. Quenched with water and extracted with EtOAc, dried and evaporated to give compound 2 (0.36 g, 100%). LC-MS: m/z=104.0 [M+H]⁺

### tert-butyl 4-hydroxytetrahydrofuran-3-ylcarbamate (3)

A solution of compound 2 (360 g, 3.5 mmol) in THF (10 mL) was added (Boc)₂O (1.14 g, 5.25 mmol) and NaOH (2N, 2 mL), then stirred at RT for 1 h. Quenched with water and extracted with EtOAc, dried and evaporated, purified by combiflash (methanol:DCM=1:20) to give compound 3 (0.2 g, 28%). LC-MS: m/z=104.0 [M+H-Boc]⁺

The procedure from Example 214 was used for step 4, step 5 and step 6, substituting tert-butyl 4-hydroxytetrahydrofuran-3-ylcarbamate.

### 2-(5-(4-aminotetrahydrofuran-3-ylamino)-2′,4′-dioxo-2,3-dihydrospiro[indene-1,5′-ozazolidine]-3′-yl)-N—((S)-1-cyclopropylethyl)-N-(4-fluorobenzyl)acetamide

LC-MS: m/z=537.5 [M+H]⁺ at RT 3.96 (96.3% purity). ¹H NMR (400 MHz, DMSO-d₆) δ 8.23 (s, 2H), 7.91 (s, 2H), 7.49 (m, 1H), 7.35-7.12 (m, 4H), 6.75-6.60 (m, 2H), 4.78 (s, 2H), 4.75-4.15 (m, 4H), 4.13-3.50 (m, 8H), 3.38 (m, 1H), 3.10-2.96 (m, 2H), 2.65-2.40 (m, 2H), 1.25-1.12 (m, 3H), 0.96 (m, 1H), 0.61-0.12 (m, 4H).

### Example 232

### 2-(4-bromophenyl)-1,3-dioxolane (1)

To a solution of 4-bromobenzaldehyde SM 1 (1.0 g, 5 mmol) in toluene (50 mL) was added ethane-1,2-diol (3.35 g, 50 mmol) and 4-methylbenzenesulfonic acid(86 mg, 0.05 mmol), then the reaction mixture was stirred at reflux for 15 h. Quenched with water and extracted with EtOAc, dried and evaporated, purified by combiflash to give compound 1 (1 g, 87%).

### 2-phenyl-1,3-dioxolane-d1 (2)

To a solution of compound 1 (0.5 g, 2.2 mmol) in THF (10 mL) was added n-BuLi (1.2 mL, 2.6 mmol) at −78° C. under N₂, stirred at −78° C. for 30 min. D₂O (90 mg, 4.4 mmol) was added at −78° C., stirred at rt for 2 h. Quenched with water and extracted with EtOAc, dried and evaporated, purified by combiflash (petroleum ether:ethyl acetate=5:1) to give compound 2 (0.2 g, 60%). LC-MS: m/z=152.1 [M+H]⁺

### benzaldehyde-d1 (3)

A solution of compound 2 (0.2 g, 1.3 mmol) in DCM(10 mL) was added FeCl₃-6H₂O (1.75 g, 6.5 mmol), then stirred at RT for 1 h. Quenched with water and extracted with DCM, dried and evaporated, purified by combiflash (petroleum ether:ethyl acetate=3:1) to give compound 3 (0.1 g, 72%).

### (S)—N-benzyl-1-cyclopropylethanamine-d1 (4)

To a stirring solution of compound 3 (0.1 g, 0.9 mmol) and (S)-1-cyclopropylethanamine (77 mg, 0.9 mmol) in MeOH (10 mL) was added NaBH₄ (72 mg, 1.8 mmol). The resulting reaction mixture was stirred at RT for 16 h. The reaction mixture was concentrated, EtOAc was added and washed with water and brine, dried and evaporated, purified by combiflash (methanol:DCM=1:20) to give compound 4 (100 mg, 63%). LC-MS: m/z=177.2 [M+H]⁺

### N-benzyl-N—((S)-1-cyclopropylethyl)-2-(5-(3-methylureido)-2′,4′-dioxo-2,3-dihydrospiro[indene-1,5′-ozazolidine]-3′-yl)acetamide-d1 (5)

A solution of intermediate 50 (50 mg, 0.15 mmol) and compound 4 (27 mg, 0.15 mmol) in DCM (5 mL) was added T₃P (95 mg, 0.3 mmol), then stirred at rt for 2 h. The resulting reaction mixture was quenched with water and extracted with DCM, dried and evaporated, purified by combiflash (methanol:DCM=1:20) to give compound 232-1 (30 mg, 41%) as a white solid. LC-MS: m/z 492.0 [M+H]⁺ at RT 4.73 (98.4% purity). ¹H NMR (301 MHz, DMSO-d₆) δ 8.71 (s, 1H), 7.60-7.13 (m, 7H), 6.09 (s, 1H), 4.81-4.13 (m, 5H), 3.76 (s, 1H), 3.15-3.25 (m, 2H), 2.73-2.55 (m, 5H), 1.25-1.10 (m, 3H), 1.15-1.00 (m, 1H), 0.60-0.45 (m, 1H), 0.40-0.25 (m, 3H).

### Example 233

### N-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)-2-methyl-2-(methylamino)propanamide

To a solution of intermediate 40 (100 mg, 0.22 mmol) and 2-methyl-2-(methylamino)propanoic acid (26 mg, 0.22 mmol) in DMF (10 mL) was added HATU (100 mg, 0.26 mmol) and DIPEA (0.85 mg, 0.66 mmol), then the reaction mixture was stirred at RT for 16 h under N₂. Quenched with water and extracted with EtOAc, washed with water and brine. Dried and concentrated under reduced pressure to obtain crude product, which was purified by Prep-HPLC, eluting with a gradient of CH₃CN/H₂O (containing 0.1% TFA) from 10/90 to 70/30 to give the desired product compound 233-1 (30 mg, 25%) as a white solid. LC-MS: m/z=551.3 [M+H]⁺ at RT 4.19 (99.2% purity). ¹H NMR (400 MHz, CD₃OD) δ 7.76 (s, 1H), 7.6-7.45 (m, 3H), 7.32 (dd, J 5=8.4, 5.5 Hz, 1H), 7.15 (t, J=8.7 Hz, 1H), 7.03 (t, J=8.7 Hz, 1H), 4.84-4.38 (m, 4H), 3.90-3.81 (m, 1H), 3.29-3.09 (m, 2H), 2.91-2.77 (m, 1H), 2.72 (s, 3H), 2.68-2.53 (m, 1H), 1.30 (dd, J=34.4, 6.7 Hz, 3H), 1.05-0.90 (m, 1H), 0.70-0.53 (m, 1H), 0.48-0.20 (m, 3H).

### Example 234

### tert-butyl (2R)-1-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-ylamino)-3-methyl-1-oxobutan-2-ylcarbamate (1)

To a stirring solution of intermediate 43 (3.3 g, 7.9 mmol) and intermediate 44 (2.5 g, 7.9 mmol) in DMF (10 mL) was added K₂CO₃ (1.6 g, 11.85 mmol) and stirred at RT for 1 h. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 50% EtOAc in PE to afford compound 1 (4.5 g, 88%) as a white solid. LC-MS: m/z=551.3 [M+H-Boc]⁺

### (2R)-2-amino-N-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)-3-methylbutanamide

To a stirring solution of compound 1 (4.5 g, 6.3 mmol) in DCM (20 mL) was added TFA (10 mL). The reaction mixture was stirred at room temperature for 1 h. After consumption of the starting material (by TLC), the reaction mixture was concentrated under reduced pressure to obtain crude product, which was purified by silica gel column chromatography eluting with 10% MeOH in DCM to afford compound 234-1 (3.4 g, 81%) as a white solid. LC-MS: m/z=551.3 [M+H]⁺ at RT 4.42 (96.4% purity). ¹H NMR (301 MHz, DMSO-d₆) δ 10.69 (s, 1H), 8.29 (s, 2H), 7.73 (s, 1H), 7.51-7.41 (m, 3H), 7.31-7.20 (m, 2H), 7.11 (t, J=8.8 Hz, 1H), 4.77 (s, 1H), 4.67-4.59 (m, 1H), 4.53-4.25 (m, 2H), 3.81-3.72 (m, 1.5H), 3.42-3.33 (m, 0.5H), 3.18-2.99 (m, 2H), 2.77-2.52 (m, 2H), 2.23-2.13 (m, 1H), 1.26-1.10 (m, 3H), 0.98 (d, J=4.5 Hz, 6H), 0.52-0.17 (m, 5H).

### Example 235

### N-benzyl-N—((S)-1-cyclopropylethyl)-2-((S)-5-(3-methylureido)-2′,4′-dioxo-2,3-dihydrospiro[indene-1,5′-ozazolidin]-3′-yl)acetamide

To a stirring solution of intermediate 18 (433 mg, 1 mmol) in DCM (20 mL) was added triphosgene (149 mg, 0.5 mmol) followed by Et₃N (304 mg, 3 mmol) at 0° C. The reaction mixture was stirred at room temperature for 1.5 h. Then methanamine (2M in THF, 2.5 mL, 5 mmol) was added and stirred at room temperature for 0.5 h. After consumption of the starting material (by TLC), the reaction mixture was diluted with water and DCM. Combined organic extracts were dried over anhydrous Na₂SO₄ and concentrated under reduced pressure to obtain crude product (343 mg, 70%), which was purified by SFC (column: CHIRAL CELL OD-H; Manufacturer: DAICEL CHIRAL TECHNOLOGIES (CHINA) CO., LTD; EtOH/CO₂ (3:7, 0.2% FA and 0.2% diethylamine in EtOH)) to afford compound 235-1 (89 mg, 52%) and compound 235-2 (92 mg, 54%) as a white solid. LC-MS: m/z=491.0 [M+H]⁺ at RT 4.68 (100% purity). ¹H NMR (301 MHz, CD₃Cl₃) δ 7.52-7.28 (m, 5H), 7.25-6.95 (m, 3H), 4.73-4.69 (m, 2H), 4.34 (s, 2H), 4.05-3.83 (m, 1.5H), 3.39-3.07 (m, 2.5H), 2.78 (s, 3H), 2.59-2.48 (m, 1H), 1.41-1.28 (m, 3H), 0.95-0.83 (m, 1H), 0.58-0.24 (m, 4H).

### N-benzyl-N—((S)-1-cyclopropylethyl)-2-((R)-5-(3-methylureido)-2′,4′-dioxo-2,3-dihydrospiro[indene-1,5′-ozazolidin]-3′-yl)acetamide

LC-MS: m/z=491.0 [M+H]⁺ at RT 8.74 (100% purity). ¹H NMR (301 MHz, CD₃Cl₃) δ 7.54-7.27 (m, 5H), 7.23-6.92 (m, 3H), 4.75-4.69 (m, 2H), 4.51-4.33 (m, 2H), 4.07-3.82 (m, 1.5H), 3.33-3.04 (m, 2.5H), 2.79 (s, 3H), 2.57-2.45 (m, 1H), 1.38-1.25 (m, 3H), 0.89-0.74 (m, 1H), 0.57-0.22 (m, 4H).

### Example 236

### tert-butyl 3-(3′-(2-(benzyl((S)-1-cyclopropylethyl)amino)-2-oxoethyl)-2′,4′-dioxo-2,3-dihydrospiro[indene-1,5′-ozazolidine]-5-ylcarbamoyl)-3-methylazetidine-1-carboxylate (1)

To a stirring solution of intermediate 18 (87 mg, 0.2 mmol) and 1-(tert-butoxycarbonyl)-3-methylazetidine-3-carboxylic acid SM 1 (43 mg, 0.2 mmol) in DCM (10 mL) was added Et₃N (40 mg, 0.4 mmol) followed by T₃P (50% in EtOAc) (254 mg, 0.4 mmol) and stirred at RT for 1 h. The reaction mixture was diluted with water and extracted with DCM. Combined organic extracts were dried over anhydrous Na₂SO₄ and concentrated under reduced pressure to obtain crude product, which was purified by silica gel column chromatography eluting with 5% MeOH in DCM to afford compound 1 (80 mg, 63%) as a light yellow solid. LC-MS: m/z 631.2 [M+H]⁺

### N-(3′-(2-(benzyl((S)-1-cyclopropylethyl)amino)-2-oxoethyl)-2′,4′-dioxo-2,3-dihydrospiro[indene-1,5′-ozazolidine]-5-yl)-3-methylazetidine-3-carboxamide

To a stirring solution of compound 1 (80 mg, 0.13 mmol) in DCM (6 mL) was added TFA (2 mL). The reaction mixture was stirred at room temperature for 2 h. After consumption of the starting material (by TLC), the reaction mixture was concentrated under reduced pressure to obtain crude product, which was purified by Prep-HPLC, eluting with a gradient of CH₃CN/H₂O (containing 0.1% TFA) from 10/90 to 80/20 to give the desired product compound 236-1 (45 mg, 65%) as TFA salt. LC-MS: m/z=531.2 [M+H]⁺ at RT 3.96 (100% purity). ¹H NMR (301 MHz, DMSO-d₆) δ 10.12 (s, 1H), 7.75 (s, 1H), 7.65-7.30 (m, 5H), 7.35-7.12 (m, 2H), 4.79 (s, 1H), 4.77-4.40 (m, 3H), 4.39-4.22 (m, 3H), 3.84-3.72 (m, 2.5H), 3.45-3.33 (m, 0.5H), 3.27-2.95 (m, 2H), 2.76-2.52 (m, 2H), 1.61 (d, J=2.1 Hz, 3H), 1.29-1.09 (m, 3H), 1.03-0.91 (m, 1H), 0.67-0.07 (m, 4H).

### Example 237

### tert-butyl 3-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-ylcarbamoyl)-3-methylazetidine-1-carboxylate (1)

To a stirring solution of intermediate 40 (168 mg, 0.37 mmol) and SM 1 (80 mg, 0.37 mmol) in DCM (20 mL) was added Et₃N (75 mg, 0.8 mmol) followed by T₃P (50% in EtOAc) (470 mg, 0.8 mmol) and stirred at RT for 1 h. The reaction mixture was diluted with water and extracted with DCM. Combined organic extracts were dried over anhydrous Na₂SO₄ and concentrated under reduced pressure to obtain crude product, which was purified by silica gel column chromatography eluting with 5% MeOH in DCM to afford compound 1 (100 mg, 41%) as a light yellow solid. LC-MS: m/z=549.3 [M+H-Boc]⁺

### N-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)-3-methylazetidine-3-carboxamide

To a stirring solution of compound 1 (100 mg, 0.15 mmol) in DCM (10 mL) was added TFA (2 mL). The reaction mixture was stirred at room temperature for 2 h. After consumption of the starting material (by TLC), the reaction mixture was concentrated under reduced pressure to obtain crude product, which was purified by Prep-HPLC, eluting with a gradient of CH₃CN/H₂O (containing 0.1% TFA) from 10/90 to 70/30 to give the desired product compound 237-1 (45 mg, 45%) as a TFA salt. LC-MS: m/z=549.0 [M+H]⁺ at RT 4.00 (100% purity). ¹H NMR (400 MHz, DMSO-d₆) δ 10.08 (s, 1H), 7.76 (s, 1H), 7.60-7.39 (m, 3H), 7.31-7.21 (m, 2H), 7.12 (t, J=8.9 Hz, 1H), 4.78 (s, 1H), 4.69-4.58 (m, 1H), 4.51-4.41 (m, 1H), 4.39-4.27 (m, 1H), 3.81-3.70 (m, 1.5H), 3.48-3.34 (m, 4.5H), 3.19-3.05 (m, 2H), 2.71-2.58 (s, 2H), 1.61 (d, J=1.9 Hz, 3H), 1.30-1.10 (m, 3H), 1.02-0.91 (m, 1H), 0.55-0.14 (m, 4H).

### Example 238

### tert-butyl (1R)-1-cyclopropyl-2-(3′-(2-((4-fluorobenzyl)((S)-1,1,1-trifluoropropan-2-yl)amino)-2-oxoethyl)-2′,4′-dioxo-2,3-dihydrospiro[indene-1,5′-ozazolidine]-5-ylamino)-2-oxoethylcarbamate (1)

To a stirring solution of intermediate 42 (155 mg, 0.23 mmol) and SM 1 (50 mg, 0.23 mmol) in DCM (20 mL) was added Et₃N (47 mg, 0.46 mmol) followed by T₃P (50% in EtOAc) (292 mg, 0.46 mmol) and stirred at RT for 1 h. The reaction mixture was diluted with water and extracted with DCM. Combined organic extracts were dried over anhydrous Na₂SO₄ and concentrated under reduced pressure to obtain crude product, which was purified by silica gel column chromatography eluting with 5% MeOH in DCM to afford compound 1 (45 mg, 29%) as a light yellow solid. LC-MS: m/z=577.2 [M+H-Boc]⁺

### (2R)-2-amino-2-cyclopropyl-N-(3′-(2-((4-fluorobenzyl)((S)-1,1,1-trifluoropropan-2-yl)amino)-2-oxoethyl)-2′,4′-dioxo-2,3-dihydrospiro[indene-1,5′-ozazolidine]-5-yl)acetamide

To a stirring solution of compound 1 (45 mg, 0.066 mmol) in DCM (10 mL) was added TFA (2 mL). The reaction mixture was stirred at room temperature for 2 h. After consumption of the starting material (by TLC), the reaction mixture was concentrated under reduced pressure to obtain crude product, which was purified by Prep-HPLC, eluting with a gradient of CH₃CN/H₂O (containing 0.1% TFA) from 10/90 to 70/30 to give the desired product compound 238-1 (20 mg, 44%) as a TFA salt. LC-MS: m/z=577.2 [M+H]⁺ at RT 4.056 (98.5% purity). ¹H NMR (400 MHz, CD₃OD) δ 7.78 (d, J=3.8 Hz, 1H), 7.56-7.39 (m, 3H), 7.36-6.95 (m, 3H), 5.56-5.40 (m, 1H), 5.03-4.80 (m, 2H), 4.67-4.30 (m, 2H), 3.39-3.34 (m, 1H), 3.31-3.19 (m, 2H), 2.87-2.57 (m, 2H), 1.42 (dd, J=27.9, 6.9 Hz, 3H), 1.34-1.27 (m, 1H), 0.90-0.56 (m, 4H).

### Example 239

### tert-butyl 3-(3′-(2-((4-fluorobenzyl)((S)-1,1,1-trifluoropropan-2-yl)amino)-2-oxoethyl)-2′,4′-dioxo-2,3-dihydrospiro[indene-1,5′-ozazolidine]-5-ylcarbamoyl)-3-methylazetidine-1-carboxylate (1)

To a stirring solution of intermediate 42 (50 mg, 0.1 mmol) and 1-(tert-butoxycarbonyl)-3-methylazetidine-3-carboxylic acid SM 1 (43 mg, 0.2 mmol) in DCM (10 mL) was added Et₃N (30 mg, 0.3 mmol) followed by T₃P (50% in EtOAc) (190 mg, 0.3 mmol) and stirred at RT for 1 h. The reaction mixture was diluted with water and extracted with DCM. Combined organic extracts were dried over anhydrous Na₂SO₄ and concentrated under reduced pressure to obtain crude product, which was purified by silica gel column chromatography eluting with 5% MeOH in DCM to afford compound 1 (50 mg, 73%) as a light yellow solid. LC-MS: m/z=577.2 [M+H-Boc]⁺

### N-(3′-(2-((4-fluorobenzyl)((S)-1,1,1-trifluoropropan-2-yl)amino)-2-oxoethyl)-2′,4′-dioxo-2,3-dihydrospiro[indene-1,5′-ozazolidine]-5-yl)-3-methylazetidine-3-carboxamide

To a stirring solution of compound 1 (50 mg, 0.074 mmol) in DCM (6 mL) was added TFA (2 mL). The reaction mixture was stirred at room temperature for 2 h. After consumption of the starting material (by TLC), the reaction mixture was concentrated under reduced pressure to obtain crude product, which was purified by Prep-HPLC, eluting with a gradient of CH₃CN/H₂O (containing 0.1% TFA) from 10/90 to 70/30 to give the desired product compound 239-1 (22 mg, 43%) as a TFA salt. LC-MS: m/z=577.2 [M+H]⁺ at RT 3.97 (100% purity). ¹H NMR (400 MHz, CD₃OD) δ 7.78 (s, 1H), 7.57-7.38 (m, 3H), 7.31-6.98 (m, 3H), 5.57-5.39 (m, 1H), 4.98-4.74 (m, 2H), 4.68-4.56 (m, 1H), 4.50 (d, J=11.1 Hz, 2H), 4.48-4.34 (m, 1H), 3.95 (d, J=11.1 Hz, 2H), 3.28-3.08 (m, 2H), 2.87-2.71 (m, 1H), 2.65-2.51 (m, 1H), 1.74 (s, 3H), 1.42 (dd, J=27.7, 6.9 Hz, 3H).

### Example 240

### N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-(dimethylamino)-2-methylpropanamide

To a solution of intermediate 18 (50 mg, 0.11 mmol) and 2-(dimethylamino)-2-methylpropanoic acid hydrochloride (19 mg, 0.11 mmol) in DMF (5 mL) was added HATU (50 mg, 0.13 mmol) and DIPEA (0.43 mg, 0.33 mmol), then the reaction mixture was stirred at RT for 16 h under N₂. Quenched with water, and extracted with EtOAc, washed with water and brine. Dried and concentrated under reduced pressure to obtain crude product, which was purified by Prep-HPLC, eluting with a gradient of CH₃CN/H₂O (containing 0.1% TFA) from 10/90 to 70/30 to give the desired product compound 240-1 (15 mg, 25%) as a white solid. LC-MS: m/z=547.1 [M+H]⁺ at RT 4.27 (97.6% purity). ¹H NMR (400 MHz, CD₃OD) δ 7.77 (s, 1H), 7.63-7.16 (m, 7H), 4.88-4.36 (m, 4H), 3.92-3.80 (m, 1H), 3.30-3.10 (m, 2H), 2.85-2.53 (m, 3H), 1.29 (dd, J=29.0, 6.7 Hz, 3H), 1.05-0.92 (m, 1H), 0.66-0.55 (m, 1H), 0.48-0.14 (m, 3H).

### Example 241

### (R)-2-((tert-butoxycarbonyl)amino)-3-hydroxypropanoic acid (1)

To a stirring solution of (Boc)₂O (1.09 g, 5 mmol) in CH₃OH (5 mL) was added the suspension of (R)-2-amino-3-hydroxypropanoic acid SM 1 (525 mg, 5 mmol) and Et₃N (507 mg, 5 mmol) in CH₃OH (5 mL) at RT and stirred overnight. The reaction mixture was concentrated and the residue was diluted with H₂O. Adjusted the pH to 9-10 and extracted with DCM. The water layer was adjusted the pH to 2 with 4N HCl aq. and extracted with DCM. Combined organic extracts were dried over anhydrous Na₂SO₄ and concentrated under reduced pressure to obtain crude compound 1 (950 mg, 92%) as a white solid.

### tert-butyl ((2R)-1-((3′-(2-(benzyl((S)-1-cyclopropylethyl)amino)-2-oxoethyl)-2′,4′-dioxo-2,3-dihydrospiro[indene-1,5′-ozazolidin]-5-yl)amino)-3-hydroxy-1-oxopropan-2-yl)carbamate (2)

To a stirring solution of compound 1 (41 mg, 0.2 mmol) and 4-methylmorpholine (21 mg, 0.2 mmol) in dry THF (5 mL) was added isobutyl carbonochloridate (27 mg, 0.25 mmol) slowly at −15° C. and stirred −15° C. for 15 min. Then intermediate 18 (87 mg, 0.2 mmol) was added. The resulting reaction mixture was warmed to RT and stirred overnight. After consumption of the starting material (by TLC), the reaction mixture was concentrated under reduced pressure to give crude product, which was purified by silica gel column chromatography eluting with 5% CH₃OH in DCM to afford compound 2 (70 mg, 56%) as an off-white solid. LC-MS: m/z=521.2 [M+H-Boc]⁺

### (2R)-2-amino-N-(3′-(2-(benzyl((S)-1-cyclopropylethyl)amino)-2-oxoethyl)-2′,4′-dioxo-2,3-dihydrospiro[indene-1,5′-ozazolidin]-5-yl)-3-hydroxypropanamide

To a stirring solution of compound 2 (70 mg, 0.11 mmol) in DCM (5 mL) was added TFA (0.5 mL). The reaction mixture was stirred at room temperature for 2 h. After consumption of the starting material (by TLC), the reaction mixture was concentrated under reduced pressure to obtain crude product, which was purified by Prep-HPLC, eluting with a gradient of CH₃CN/H₂O (containing 0.1% TFA) from 10/90 to 70/30 to give the desired product compound 241-1 (45 mg, 64%) as a TFA salt. LC-MS: m/z=521.1 [M+H]⁺ at RT 3.72 (98.7% purity). ¹H NMR (400 MHz, CD₃OD) δ 7.78 (s, 1H), 7.54-7.27 (m, 7H), 4.83-4.68 (m, 2H), 4.66-4.44 (m, 2H), 4.21-3.92 (m, 3H), 3.85-3.72 (m, 1H), 3.29-0.27 (m, 2H), 2.77-2.64 (m, 1H), 2.69-2.53 (m, 1H), 1.29 (dd, J=29.2, 6.8 Hz, 3H), 1.05-0.87 (m, 1H), 0.65-0.22 (m, 4H).

### Example 242

### (S)-5-((1-cyclopropylethylamino)methyl)thiazol-2-amine (1)

To a stirring solution of SM 1 (128 mg, 1 mmol) and (S)-1-cyclopropylethanamine (102 mg, 1.3 mmol) in 1,2-dichloroethane (2 mL) was added NaBH(OAc)₃ (252 mg, 24 mmol) at RT and stirred overnight. After consumption of the starting material (by TLC), the reaction mixture was quenched with H₂O and extracted with EtOAc. Combined organic extracts were dried over anhydrous Na₂SO₄ and concentrated under reduced pressure to obtain crude product, which was purified by silica gel column chromatography eluting with 10% CH₃OH in DCM to afford compound 1 (95 mg, 48%) as an off-white solid. LC-MS: m/z=198.1 [M+H]⁺

### N-((2-aminothiazol-5-yl)methyl)-N—((S)-1-cyclopropylethyl)-2-((S)-2,5-dioxo-2′,3′-dihydrospiro[imidazolidine-4,1′-indene]-1-yl)acetamide

To a stirring solution of compound 1 (47 mg, 0.24 mmol) and intermediate 9 (62 mg, 0.24 mmol) in DMF (2 mL) was added HATU (99 mg, 0.26 mmol) followed by DIPEA (38 mg, 0.29 mmol) and stirred at RT for 1 h. The reaction mixture was diluted with water and extracted with EtOAc. Combined organic extracts were dried over anhydrous Na₂SO₄ and concentrated under reduced pressure to obtain crude product, which was purified by Prep-HPLC, eluting with a gradient of CH₃CN/H₂O (containing 0.1% TFA) from 10/90 to 70/30 to give the desired product compound 242-1 (2 mg, 2%) as an off-white solid. LC-MS: m/z=440.1 [M+H]⁺ at RT 3.24 (99.4% purity). ¹H NMR (400 MHz, DMSO-d₆) δ 8.82 (s, 1H), 8.67 (br s, 2H), 7.37-7.18 (m, 5H), 4.68-4.52 (m, 2H), 4.39-4.19 (m, 2H), 3.57-3.51 (m, 0.5H), 3.36-3.31 (m, 0.5H), 3.09-3.01 (m, 2H), 2.63-2.54 (m, 1H), 2.30-2.16 (m, 1H), 1.32-1.13 (m, 3H), 1.13-0.96 (m, 1H), 0.61-0.22 (m, 4H).

### Example 243

### 2-(dimethylamino)thiazole-5-carbaldehyde (1)

To a stirring suspension of 2-aminothiazole-5-carbaldehyde SM 1 (128 mg, 1 mmol) and K₂CO₃ (346 mg, 2.5 mmol) in DMF (1 mL) was added iodomethane (284 mg, 2 mmol) and heated to 50° C. for 2 h. After consumption of the starting material (by TLC), the reaction mixture was diluted with water and extracted with EtOAc. Combined organic extracts were dried over anhydrous Na₂SO₄ and concentrated under reduced pressure to obtain crude compound 1 (155 mg, 100%) as a yellow solid, which was used for next step directly. LC-MS: m/z=157.0 [M+H]⁺

### (S)-5-((1-cyclopropylethylamino)methyl)-N,N-dimethylthiazol-2-amine (2)

To a stirring solution of compound 1 (75 mg, 0.48 mmol) and (S)-1-cyclopropylethanamine (82 mg, 0.96 mmol) in CH₃OH (10 mL) was added NaBH₄ (91 mg, 2.4 mmol) at RT and stirred for 4 h. After consumption of the starting material (by TLC), the reaction mixture was quenched with H₂O and extracted with EtOAc. Combined organic extracts were dried over anhydrous Na₂SO₄ and concentrated under reduced pressure to obtain crude compound 2 (78 mg, 72%) as an off-white solid, which was used for next step directly. LC-MS: m/z=226.1 [M+H]⁺

### N—((S)-1-cyclopropylethyl)-N-((2-(dimethylamino)thiazol-5-yl)methyl)-2-((S)-2,5-dioxo-2′,3′-dihydrospiro[imidazolidine-4,1′-indene]-1-yl)acetamide

To a stirring solution of compound 2 (52 mg, 0.23 mmol) and intermediate 9 (60 mg, 0.23 mmol) in DMF (1 mL) was added HATU (99 mg, 0.26 mmol) followed by DIPEA (38 mg, 0.29 mmol) and stirred at RT for 1 h. The reaction mixture was diluted with water and extracted with EtOAc. Combined organic extracts were dried over anhydrous Na₂SO₄ and concentrated under reduced pressure to obtain crude product, which was purified by Prep-HPLC, eluting with a gradient of CH₃CN/H₂O (containing 0.1% TFA) from 10/90 to 70/30 to give the desired product compound 243-1 (26 mg, 24%) as a white solid. LC-MS: m/z=468.0 [M+H]⁺ at RT 3.31 (97% purity). ¹H NMR (400 MHz, DMSO-d₆) δ 8.81 (d, J=17.1 Hz, 1H), 7.53-7.16 (m, 5H), 4.73-4.55 (m, 2H), 4.38-4.23 (m, 2H), 3.67-3.61 (m, 0.5H), 3.36-3.30 (m, 0.5H), 3.08 (d, J=13.4 Hz, 6H), 2.61-2.52 (m, 3H), 2.32-2.17 (m, 1H), 1.30-1.16 (m, 3H), 1.13-1.05 (m, 1H), 0.65-0.13 (m, 4H).

### Example 244

### (S)-5-((1-cyclopropylethylamino)methyl)-N,N-dimethylpyridin-2-amine (1)

To a stirring solution of 6-(dimethylamino)nicotinaldehyde SM 1 (450 mg, 3 mmol) and (S)-1-cyclopropylethanamine (281 mg, 3.3 mmol) in CH₂Cl₂ (20 mL) was added TiCl(OiPr)₃ (6.24 g, 24 mmol) at RT under N₂ (g). The reaction mixture was stirred at RT for 2 h before and NaBH(OAc)₃ (4.84 g, 24 mmol) was added. After consumption of the starting material (by TLC), the reaction mixture was quenched with H₂O and filtered. The filtrate was concentrated under reduced pressure. The obtained residue was purified by silica gel column chromatography eluting with 10% CH₃OH in DCM to afford compound 1 (0.38 g, 58%) as an off-white solid. LC-MS: m/z=220.1 [M+H]⁺

### N—((S)-1-cyclopropylethyl)-N-((6-(dimethylamino)pyridin-3-yl)methyl)-2-((S)-2,5-dioxo-2′,3′-dihydrospiro[imidazolidine-4,1′-indene]-1-yl)acetamide

To a stirring solution of compound 1 (46 mg, 0.21 mmol) and intermediate 9 (55 mg, 0.21 mmol) in DMF (2 mL) was added HATU (120 mg, 0.32 mmol) followed by DIPEA (55 mg, 0.42 mmol) and stirred at RT for 2 h. The reaction mixture was diluted with water and extracted with EtOAc. Combined organic extracts were dried over anhydrous Na₂SO₄ and concentrated under reduced pressure to obtain crude product, which was purified by Prep-HPLC, eluting with a gradient of CH₃CN/H₂O (containing 0.1% TFA) from 10/90 to 70/30 to give the desired product compound 244-1 (37 mg, 38%) as an off-white solid. LC-MS: m/z=462.0 [M+H]⁺ at RT 3.08 (99.5% purity). ¹H NMR (301 MHz, DMSO-d₆) δ 8.83 (s, 1H), 7.99-7.81 (m 2H), 7.41-7.15 (m, 5H), 4.72-4.52 (m, 2H), 4.41-4.18 (m, 2H), 3.71-3.65 (s, 0.5H), 3.39-3.29 (m, 0.5H), 3.18 (s, 6H), 3.05 (t, J=7.3 Hz, 2H), 2.63-2.53 (m, 1H), 2.29-2.21 (m, 1H), 1.20 (dd, J=42.0, 6.6 Hz, 3H), 1.01-0.92 (m, 1H), 0.63-0.10 (m, 4H).

### Example 245

### 2-(5-bromo-2′,4′-dioxo-2,3-dihydrospiro[indene-1,5′-ozazolidine]-3′-yl)-N—((S)-1-cyclopropylethyl)-N-(4-fluorobenzyl)acetamide

To a solution of intermediate 25 (2.3 g, 8 mmol) and intermediate 44 (2 g, 6.4 mmol) in DMF (20 mL) was added K₂CO₃ (2.64 g, 19.2 mmol), then the reaction mixture was stirred at RT for 2 h. Quenched with water, extracted with EtOAc, washed with water and brine, dried and concentrated under reduced pressure to obtain crude product, which was purified by combiflash (methanol:DCM=1:20) to afford compound 245-1 (2.2 g, 67%) as a white solid. LC-MS: m/z=515.0/517.0 [M+H]⁺ at RT 5.83 (91.7% purity). ¹H NMR (400 MHz, DMSO-d₆) δ 7.68 (s, 1H), 7.57 (t, J=8.1 Hz, 1H), 7.48-7.42 (m, 2H), 7.33-7.21 (m, 2H), 7.12 (t, J=8.8 Hz, 1H), 4.78 (s, 1H), 4.71-4.25 (m, 3H), 3.82-3.67 (m, 1H), 3.27-3.02 (m, 2H), 2.76-2.53 (m, 2H), 1.26-1.10 (m, 3H), 1.02-0.90 (m, 1H), 0.57-0.11 (m, 4H).

### Example 246

### tert-butyl 3-hydroxycyclohexylcarbamate (1)

To a stirring solution of cyclopropanecarbaldehyde SM 1 (2.23 g, 20 mmol) and (Boc)₂O (5.45 g, 25 mmol) in dioxane (50 mL) was TEA (3.03 g, 30 mmol). The resulting reaction mixture was stirred at RT for overnight. The mixture was concentrated and washed with water and brine, dried and evaporated to give compound 1 (3.5 g, 81%) as oil. LC-MS: m/z=116.1 [M+H-Boc]⁺

### tert-butyl 3-oxocyclohexylcarbamate (2)

To a stirring solution of DMSO (1.95 g, 25 mmol) and TEA (5.05 g, 50 mmol) in dry DCM (50 mL) was added oxalyl dichloride (1.5 g, 12 mmol) −78° C. under N₂. After stirred −78° C. for 1 h, compound 1 (2.1 g, 10 mmol) was added. The reaction mixture was stirred at RT for 4 h. Quenched with water, extracted with DCM, concentrated under reduced pressure to obtain crude product, which was purified by combiflash (methanol:DCM=1:20) to give compound 2 (1.7 g, 78%) as a white solid.

### tert-butyl 3-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-ylamino)cyclohexylcarbamate (3)

A solution of intermediate 40 (60 mg, 0.13 mmol) and compound 2 (57 mg, 0.26 mmol) in DCM (2 mL) was added TiCl(Oi-Pr)₃ (135 mg, 0.52 mmol) and, NaBH(OAc)₃ (110 mg, 0.52 mmol), then stirred at RT for 16 h. The resulting reaction mixture was quenched with water and extracted with DCM, dried and evaporated to give compound 3 (150 mg, 100%) as a solid. LC-MS: m/z=549.2 [M+H-Boc]⁺

### 2-(5-(3-aminocyclohexylamino)-2′,4′-dioxo-2,3-dihydrospiro[indene-1,5′-ozazolidine]-3′-yl)-N—((S)-1-cyclopropylethyl)-N-(4-fluorobenzyl)acetamide

A solution of compound 3 (150 mg, 0.23 mmol) in DCM (10 mL) was added TFA (0.5 mL). The reaction mixture was stirred at room temperature for 2 h. Concentrated under reduced pressure to obtain crude product, which was purified by Prep-HPLC, eluting with a gradient of CH₃CN/H₂O (containing 0.1% TFA) from 10/90 to 70/30 to give the desired product compound 246-1 (31 mg, 40%) as a white solid. LC-MS: m/z=549.1 [M+H]⁺ at RT 4.06 (100% purity). ¹H NMR (400 MHz, CD₃OD) δ 7.55-7.45 (m, 1H), 7.35-7.25 (m, 2H), 7.15 (t, J=8.7 Hz, 1H), 7.03 (t, J=8.7 Hz, 1H), 6.72-6.57 (m, 2H), 4.83-4.32 (m, 4H), 3.91-3.78 (m, 1H), 3.52-3.40 (m, 1H), 3.29-2.94 (m, 3H), 2.83-2.66 (m, 1H), 2.62-2.36 (m, 2H), 2.21-1.43 (m, 5H), 1.41-1.12 (m, 5H), 1.03-0.85 (m, 1H), 0.68-0.54 (m, 1H), 0.48-0.14 (m, 3H).

### Example 247

### ethyl 2-(tert-butoxycarbonylamino)thiazole-4-carboxylate (1)

To a stirring solution of ethyl 2-aminothiazole-4-carboxylate SM 1 (0.5 g, 3 mmol) and (Boc)₂O (0.65 g, 3 mmol) in THF (10 mL) was TEA (0.6 g, 6 mmol) and DMAP (36 mg, 0.3 mmol). The resulting reaction mixture was stirred at RT for overnight. The mixture was quenched with water and extracted with EtOAc, dried and evaporated, purified by combiflash (methanol:DCM=1:20) to give compound 1 (0.5 g, 61%). LC-MS: m/z 172.0 [M+H-Boc]⁺

### tert-butyl 4-(hydroxymethyl)thiazol-2-ylcarbamate (2)

To a stirring solution of compound 1 (0.5 g, 1.8 mmol) in dry THF (10 mL) was added LiAlH₄ (137 mg, 3.6 mmol) at 0° C. under N₂. After stirred at 0° C. for 3 h, Quenched with water, extracted with EtOAc, concentrated under reduced pressure to obtain crude product, which was purified by combiflash (methanol:DCM=1:20) to give compound 2 (0.4 g, 96%). LC-MS: m/z 131.0 [M+H-Boc]⁺

### tert-butyl 4-formylthiazol-2-ylcarbamate (3)

A solution of compound 2 (400 mg, 1.7 mmol) in DCM (10 mL) was added Dess-MaRTin (1.44 g, 3.4 mmol), then stirred at RT for 16 h. The resulting reaction mixture was quenched with water and extracted with DCM, dried and evaporated, purified by combiflash (methanol:DCM=1:20) to give compound 3 (300 mg, 75%).

### (S)-tert-butyl 4-((1-cyclopropylethylamino)methyl)thiazol-2-ylcarbamate (4)

To a stirring solution of compound 3 (0.1 g, 0.44 mmol) and (S)-1-cyclopropylethanamine (56 mg, 0.66 mmol) in MeOH (10 mL) was added NaBH₄ (35 mg, 0.88 mmol). The resulting reaction mixture was stirred at RT for 16 h. The reaction mixture was concentrated, EtOAc was added and washed with water and brine, dried and evaporated, purified by combiflash (methanol:DCM=1:20) to give compound 4 (130 mg, 100%). LC-MS: m/z=198.2 [M+H-Boc]⁺

### tert-butyl 4-((N—((S)-1-cyclopropylethyl)-2-((S)-2,5-dioxo-2′,3′-dihydrospiro[imidazolidine-4,1′-indene]-1-yl)acetamido)methyl)thiazol-2-ylcarbamate (5)

A solution of intermediate 9 (114 mg, 0.44 mmol) and compound 4 (130 mg, 0.44 mmol) in DCM (2 mL) was added T₃P (280 mg, 0.88 mmol), then stirred at RT for 2 h. The resulting reaction mixture was quenched with water and extracted with DCM, dried and evaporated, purified by combiflash (methanol:DCM=1:20) to give compound 5 (150 mg, 63%) as a white solid. LC-MS: m/z=440.2 [M+H-Boc]⁺

### N-((2-aminothiazol-4-yl)methyl)-N—((S)-1-cyclopropylethyl)-2-((S)-2,5-dioxo-2′,3′-dihydrospiro[imidazolidine-4,1′-indene]-1-yl)acetamide

A solution of compound 5 (150 mg, 0.28 mmol) in DCM (10 mL) was added TFA (0.5 mL). The reaction mixture was stirred at room temperature for 2 h. Concentrated under reduced pressure to obtain crude product, which was purified by Prep-HPLC, eluting with a gradient of CH₃CN/H₂O (containing 0.1% TFA) from 10/90 to 70/30 to give the desired product compound 247-1 (70 mg, 57%) as a white solid. LC-MS: m/z=440.0 [M+H]⁺ at RT 3.25 (100% purity). ¹H NMR (400 MHz, CD₃OD) δ 7.43-7.21 (m, 5H), 6.71 (s, 1H), 4.71-4.32 (m, 5H), 3.46-3.35 (m, 1H), 3.23-3.06 (m, 3H), 2.77-2.68 (m, 1H), 2.40-2.30 (−m, 1H), 1.42 (d, J=6.6 Hz, 3H), 1.29-1.06 (m, 1H), 0.77-0.68 (m, 1H), 0.56-0.22 (m, 4H).

### Example 248

### tert-butyl 3-(2,2-dimethyl-4,6-dioxo-1,3-dioxane-5-carbonyl)azetidine-1-carboxylate (1)

To a stirring solution of ethyl 1-(tert-butoxycarbonyl)azetidine-3-carboxylic acid SM 1 (2.4 g, 12 mmol) and 2,2-dimethyl-1,3-dioxane-4,6-dione (1.72 g, 12 mmol) in DCM (50 mL) was DCC (2.44 g, 12 mmol) and DMAP (2.92 mg, 24 mmol). The resulting reaction mixture was stirred at RT for overnight. The mixture was quenched with water and extracted with DCM, dried and evaporated to give compound 1 (2.8 g, 70%). LC-MS: m/z=228.0 [M+H-Boc]⁺

### tert-butyl 3-acetylazetidine-1-carboxylate (2)

To a stirring solution of compound 1 (2.8 g, 8.6 mmol) in dry dioxane (20 mL) was added AcOH (1.5 mL) and H₂O (0.75 mL), then stirred at 150° C. for 30 min with microwave heating. Quenched with water, extracted with EtOAc, concentrated under reduced pressure to obtain crude product, which was purified by combiflash (methanol:DCM=1:20) to give compound 2 (1.2 g, 70%). LC-MS: m/z=100.0 [M+H-Boc]⁺

### tert-butyl 3-(1-(benzylamino)ethyl)azetidine-1-carboxylate (3)

A solution of compound 2 (400 mg, 2 mmol) and phenylmethanamine (260 mg, 2.4 mmol) in MeOH (10 mL) was added Sodium triacetoxyborohydride (636 mg, 3 mmol), then stirred at RT for 16 h. The resulting reaction mixture was quenched with water and extracted with EtOAc, dried and evaporated, purified by combiflash to give compound 3 (43 mg, 74%). LC-MS: m/z=191.0 [M+H-Boc]⁺

### tert-butyl 3-(1-(N-benzyl-2-(5-(3-methylureido)-2′,4′-dioxo-2,3-dihydrospiro[indene-1,5′-ozazolidine]-3′-yl)acetamido)ethyl)azetidine-1-carboxylate (4)

A solution of intermediate 50 (100 mg, 0.3 mmol) and compound 3 (105 mg, 0.36 mmol) in DCM (10 mL) was added T₃P (0.5 mL, 50% in EtOAc), then stirred at RT for 2 h. The resulting reaction mixture was quenched with water and extracted with DCM, dried and evaporated, purified by combiflash (methanol:DCM=1:20) to give compound 4 (130 mg, 72%) as a white solid. LC-MS: m/z=506.2 [M+H-Boc]⁺

### N-(1-(azetidin-3-yl)ethyl)-N-benzyl-2-(5-(3-methylureido)-2′,4′-dioxo-2,3-dihydrospiro[indene-1,5′-ozazolidine]-3′-yl)acetamide

A solution of compound 4 (130 mg, 0.21 mmol) in DCM (10 mL) was added TFA (0.5 mL). The reaction mixture was stirred at room temperature for 2 h. Concentrated under reduced pressure to obtain crude product, which was purified by Prep-HPLC, eluting with a gradient of CH₃CN/H₂O (containing 0.1% TFA) from 10/90 to 70/30 to give the desired product compound 248-1 (90 mg, 81%) as a white solid. LC-MS: m/z=506.0 [M+H]⁺ at RT 3.75 (97.7% purity). ¹H NMR (400 MHz, DMSO-d₆) δ 8.90-8.75 (m, 1H), 8.65-8.35 (m, 2H), 8.06 (s, 1H), 7.61-7.15 (m, 7H), 6.61-6.11 (m, 1H), 5.13-4.11 (m, 5H), 4.01-3.46 (m, 5H), 3.21-2.86 (m, 3H), 2.75-2.55 (m, 4H), 1.34-0.95 (m, 3H).

### Example 249

### 2H-spiro[benzo[b]thiophene-3,4′-imidazolidine]-2′,5′-dione,1,1-dioxide (1)

A solution of SM 1 (150 mg, 0.54 mmol) and KCN (160 mg, 1.6 mmol) in formamide (10 mL) was added (NH₄)₂CO₃ (395 mg, 2.7 mmol), then stirred at 40° C. for 16 h. The resulting reaction mixture was quenched with water and extracted with EtOAc, dried and evaporated, purified by combiflash (methanol:DCM=1:20) to give compound 1 (90 mg, 66%) as a white solid. LC-MS: m/z=253.1 [M+H]⁺

### N—((S)-1-cyclopropylethyl)-2-(2′,5′-dioxo-2H-spiro[benzo[b]thiophene, 1,1-dioxide-3,4′-imidazolidine]-1′-yl)-N-(4-fluorobenzyl)acetamide

To a solution of compound 1 (50 mg, 0.2 mmol) and intermediate 44 (62 mg, 0.2 mmol) in DMF (5 mL) was added K₂CO₃ (54 mg, 0.4 mmol), then the reaction mixture was stirred at RT for 24 h. Quenched with water, extracted with EtOAc, washed with water and brine, dried and concentrated under reduced pressure to obtain crude product, which was purified by Prep-HPLC, eluting with a gradient of CH₃CN/H₂O (containing 0.1% TFA) from 10/90 to 70/30 to give the desired product compound 249-1 (50 mg, 51%) as a white solid. LC-MS: m/z=486.0 [M+H]⁺ at RT 4.33 (97.9% purity). ¹H NMR (400 MHz, DMSO-d₆) δ 9.42-9.31 (m, 1H), 7.95-7.75 (m, 4H), 7.45-7.02 (m, 5H), 4.75-3.60 (m, 7H), 1.25-1.10 (m, 3H), 1.00-0.82 (m, 1H), 0.55-0.15 (m, 4H).

### Example 250

### (S)-5-((1-cyclopropylethylamino)methyl)pyridin-2-ol (1)

A solution of 6-hydroxynicotinaldehyde SM 1 (192 mg, 4 mmol) and (S)-1-cyclopropylethanamine (170 mg, 2 mmol) in DCM (5 mL) and EtOH (5 mL) was added NaBH₃CN (620 mg, 10 mmol), then stirred at RT for 16 h. The resulting reaction mixture was quenched with water and extracted with DCM, dried and evaporated, purified by combiflash (methanol:DCM=1:20) to give compound 1 (300 mg, 78%). LC-MS: m/z=193.1 [M+H]⁺

### N—((S)-1-cyclopropylethyl)-2-((S)-2,5-dioxo-2′,3′-dihydrospiro[imidazolidine-4,1′-indene]-1-yl)-N-((6-hydroxypyridin-3-yl)methyl)acetamide

To a solution of compound 1 (48 mg, 0.25 mmol) and intermediate 9 (60 mg, 0.23 mmol) in DMF (5 mL) was added DIPEA (89 mg, 0.69 mmol) and HATU (174 mg, 0.46 mmol), then the reaction mixture was stirred at RT for 2 h. Quenched with water, extracted with EtOAc, washed with water and brine, dried and concentrated under reduced pressure to obtain crude product, which was purified by Prep-HPLC, eluting with a gradient of CH₃CN/H₂O (containing 0.1% TFA) from 10/90 to 70/30 to give the desired product compound 250-1 (25 mg, 25%) as a white solid. LC-MS: m/z=435.1 [M+H]⁺ at RT 3.08 (99.23% purity). ¹H NMR (400 MHz, CD₃OD) δ 7.85-7.18 (m, 6H), 6.76-6.55 (m, 1H), 4.69-4.30 (m, 4H), 3.84-3.68 (m, 1H), 3.25-3.06 (m, 2H), 2.80-2.64 (m, 1H), 2.45-2.26 (m, 1H), 1.47-1.18 (m, 3H), 1.11-0.87 (m, 1H), 0.72-0.58 (m, 1H), 0.52-0.21 (m, 3H).

### Example 251

### 6-methoxynicotinaldehyde (1)

A solution of 5-bromo-2-methoxypyridine SM 1 (1.04 g, 5.5 mmol) in THF (20 mL) was added n-BuLi (5.5 mL, 5.5 mmol) −78° C. under N₂, stirred −78° C. for 0.5 h. Then DMF (1.5 mL) was added and stirred −78° C. for 2 h. The reaction was quenched with water and extracted with EtOAc, dried and evaporated, purified by combiflash (petroleum ether:ethyl acetate=1:1) to give compound 1 (150 mg, 20%).

### (S)-1-cyclopropyl-N-((6-methoxypyridin-3-yl)methyl)ethanamine (1)

A solution of compound 1 (70 mg, 0.5 mmol) and (S)-1-cyclopropylethanamine (43 mg, 0.5 mmol) in MeOH (5 mL) was added NaBH₄ (95 mg, 2.5 mmol), then stirred at RT for 16 h. The resulting reaction mixture was quenched with water and extracted with DCM, dried and evaporated, purified by combiflash (methanol:DCM=1:20) to give compound 2 (100 mg, 98%). LC-MS: m/z=207.1 [M+H]⁺

### N—((S)-1-cyclopropylethyl)-2-((S)-2,5-dioxo-2′,3′-dihydrospiro[imidazolidine-4,1′-indene]-1-yl)-N-((6-methoxypyridin-3-yl)methyl)acetamide

To a solution of compound 2 (40 mg, 0.2 mmol) and intermediate 9 (54 mg, 0.2 mmol) in DMF (5 mL) was added DIPEA (31 mg, 0.24 mmol) and HATU (83 mg, 0.22 mmol), then the reaction mixture was stirred at RT for 1 h. Quenched with water, extracted with EtOAc, washed with water and brine, dried and concentrated under reduced pressure to obtain crude product, which was purified by Prep-HPLC, eluting with a gradient of CH₃CN/H₂O (containing 0.1% TFA) from 10/90 to 70/30 to give the desired product compound 251-1 (74 mg, 86%) as a white solid. LC-MS: m/z=449.2 [M+H]⁺ at RT 3.76 (97.8% purity). ¹H NMR (400 MHz, CD₃OD) δ 8.32-7.91 (m, 2H), 7.47-6.94 (m, 5H), 4.83-4.28 (m, 4H), 4.11-3.93 (m, 3H), 3.44-3.35 (m, 1H), 3.25-3.04 (m, 2H), 2.85-2.65 (m, 1H), 2.44-2.25 (m, 1H), 1.46-1.19 (m, 3H), 1.07-0.87 (m, 1H), 0.71-0.55 (m, 1H), 0.52-0.18 (m, 3H).

### Example 252

### 2-amino-N-(3′-(2-((4-fluorobenzyl)((S)-1,1,1-trifluoropropan-2-yl)amino)-2-oxoethyl)-2′,4′-dioxo-2,3-dihydrospiro[indene-1,5′-ozazolidine]-5-yl)-2-methylpropanamide

To a solution of intermediate 42 (70 mg, 0.15 mmol) and 2-amino-2-methylpropanoic acid (31 mg, 0.3 mmol) in DCM (5 mL) was added TEA (46 mg, 0.45 mmol) and T₃P (71 mg, 0.225 mmol), then the reaction mixture was stirred at RT for 2 h. Quenched with water, extracted with DCM, washed with water and brine, dried and concentrated under reduced pressure to obtain crude product, which was purified by Prep-HPLC, eluting with a gradient of CH₃CN/H₂O (containing 0.1% TFA) from 10/90 to 70/30 to give the desired product compound 252-1 (2 mg, 3%) as a white solid. LC-MS: m/z=565.1 [M+H]⁺ at RT 3.98 (99.3% purity). ¹H NMR (400 MHz, CD₃OD) δ 7.76 (s, 1H), 7.25-7.03 (m, 6H), 4.55-5.38 (m, 1H), 4.95-4.25 (m, 4H), 3.30-3.15 (m, 2H), 3.85-2.55 (m, 2H), 1.72 (s, 6H), 1.50-1.28 (m, 3H).

### Example 253

### (S)—N-(4-fluorobenzyl)-1-methoxypropan-2-amine (1)

A solution of 4-fluorobenzaldehyde SM 1 (0.3 g, 2.5 mmol) and (S)-1-methoxypropan-2-amine (245 mg, 2.75 mmol) in MeOH (10 mL) was stirred at RT for 2 h. Then Sodium triacetoxyborohydride (583 mg, 2.75 mmol) was added and stirred at RT for overnight. Quenched with water, extracted with EtOAc, concentrated under reduced pressure to obtain crude product, which was purified by combiflash (methanol:DCM=1:20) to give compound 1 (0.4 g, 81%). LC-MS: m/z=198.1 [M+H]⁺

### (S)-2-bromo-N-(4-fluorobenzyl)-N-(1-methoxypropan-2-yl)acetamide (2)

To a stirring solution of compound 1 (0.12 g, 0.6 mmol) in dry DCM (20 mL) was added DIPEA (232 mg, 1.8 mmol) and bromoacetyl bromide (182 mg, 0.9 mmol), then stirred at RT for 3 h. Quenched with water, extracted with DCM, concentrated under reduced pressure to obtain crude product, which was purified by combiflash (petroleum ether:ethyl acetate=1:1) to give compound 2 (0.1 g, 52%). LC-MS: m/z=318.1/320.1 [M+H]⁺

### 2-(5-acetamido-2′,4′-dioxo-2,3-dihydrospiro[indene-1,5′-ozazolidine]-3′-yl)-N-(4-fluorobenzyl)-N—((S)-1-methoxypropan-2-yl)acetamide (3)

To a solution of compound 2 (0.1 g, 0.3 mmol) and intermediate 8 (78 mg, 0.3 mmol) in DMF (5 mL) was added K₂CO₃ (82 mg, 0.6 mmol), then the reaction mixture was stirred at RT for 2 h. Quenched with water, extracted with EtOAc, washed with water and brine, dried and concentrated under reduced pressure to obtain crude product, which was purified by combiflash (methanol:DCM=1:20) to afford compound 3 (120 mg, 77%) as a white solid. LC-MS: m/z=497.2 [M+H]⁺

### 2-(5-amino-2′,4′-dioxo-2,3-dihydrospiro[indene-1,5′-ozazolidine]-3′-yl)-N-(4-fluorobenzyl)-N—((S)-1-methoxypropan-2-yl)acetamide (4)

A solution of compound 3 (120 mg, 0.24 mmol) in MeOH (10 mL) was added HCl (1 mL), then stirred at RT 60° C. for 2 h. The resulting reaction mixture was quenched with Na₂CO₃ and extracted with DCM, dried and evaporated, purified by combiflash (methanol:DCM=1:20) to give compound 4 (84 mg, 77%) as a white solid. LC-MS: m/z=456.2 [M+H]⁺

### N-(4-fluorobenzyl)-N—((S)-1-methoxypropan-2-yl)-2-(5-(3-methylureido)-2′,4′-dioxo-2,3-dihydrospiro[indene-1,5′-ozazolidine]-3′-yl)acetamide

A solution of compound 4 (84 mg, 0.18 mmol) in THF (10 mL) was added TEA (54 mg, 0.54 mmol) and triphosgen (26 mg, 0.09 mmol) at 0° C. under N₂. The reaction mixture was stirred at room temperature for 0.5 h. Then MeNH₂ (0.27 mL, 0.54 mmol) was added and stirred at RT for 1 h. Quenched with water and extracted with EtOAc, dried and concentrated under reduced pressure to obtain crude product, which was purified by Prep-HPLC, eluting with a gradient of CH₃CN/H₂O (containing 0.1% TFA) from 10/90 to 70/30 to give the desired product compound 253-1 (40 mg, 42%) as a white solid. LC-MS: m/z=513.1 [M+H]⁺ at RT 4.06 (98.1% purity). ¹H NMR (400 MHz, DMSO-d₆) 8.70 (s, 1H), 7.66-7.03 (m, 7H), 6.08 (s, 1H), 4.75-4.25 (m, 5H), 3.44-3.35 (m, 1H), 3.32-3.24 (m, 3H), 3.21-2.93 (m, 5H), 2.65 (s, 3H), 1.14-1.01 (m, 3H).

### Example 254

### 3-(tert-butoxycarbonylamino)oxetane-3-carboxylic acid (1)

A solution of 3-aminooxetane-3-carboxylic acid SM 1 (0.23 g, 2 mmol) and (Boc)₂O (872 mg, 4 mmol) in MeOH (10 mL) and TEA (1 mL) was NaOH (2 mL, 1N in water), stirred at RT for 16 h. Quenched with water, extracted with EtOAc, then the water layer was adjusted pH to 2˜3 with HCl (2N), extracted with EtOAc and dried, concentrated under reduced pressure to give compound 1 (0.38 g, 87%). LC-MS: m/z=118.1 [M+H-Boc]⁺

### tert-butyl 3-(3′-(2-(benzyl((S)-1-cyclopropylethyl)amino)-2-oxoethyl)-2′,4′-dioxo-2,3-dihydrospiro[indene-1,5′-ozazolidine]-5-ylcarbamoyl)oxetan-3-ylcarbamate (2)

To a solution of intermediate 18 (86 mg, 0.2 mmol) and compound 1 (44 mg, 0.2 mmol) in DCM (10 mL) was added TEA (60 mg, 0.6 mmol) and T₃P (127 mg, 0.4 mmol), then the reaction mixture was stirred at RT for 2 h. Quenched with water, extracted with DCM, washed with water and brine, dried and concentrated under reduced pressure to obtain crude product, which was purified by combiflash (methanol:DCM=1:20) to give compound 2 (90 mg, 71%) as a white solid. LC-MS: m/z=633.1 [M+H]⁺

### 3-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)oxetane-3-carboxamide

A solution of compound 2 (90 mg, 0.14 mmol) in DCM (10 mL) was added TFA (0.5 mL). The reaction mixture was stirred at room temperature for 2 h. Concentrated under reduced pressure to obtain crude product, which was purified by Prep-HPLC, eluting with a gradient of CH₃CN/H₂O (containing 0.1% TFA) from 10/90 to 70/30 to give the desired product compound 254-1 (40 mg, 54%) as a white solid. LC-MS: m/z=533.2 [M+H]⁺ at RT 4.15 (98.2% purity). ¹H NMR (400 MHz, CD₃OD) δ 7.82-7.76 (m, 1H), 7.67-7.14 (m, 7H), 5.25-5.11 (m, 2H), 4.85-4.36 (m, 6H), 3.95-3.80 (m, 1H), 3.30-3.07 (m, 2H), 2.91-2.74 (m, 1H), 2.70-2.51 (m, 1H), 1.40-1.18 (m, 3H), 1.07-0.89 (m, 1H), 0.70-0.55 (m, 1H), 0.47-0.20 (m, 3H).

### Example 255

### ethyl 2-(dimethylamino)thiazole-4-carboxylate (1)

To a stirring suspension of 2-aminothiazole-5-carbaldehyde SM 1 (400 mg, 2.32 mmol) and K₂CO₃ (641 mg, 4.64 mmol) in DMF (10 mL) was added iodomethane (724 mg, 5.1 mmol) and heated to 50° C. overnight. After consumption of the starting material (by TLC), the reaction mixture was diluted with water and extracted with EtOAc. Combined organic extracts were dried over anhydrous Na₂SO₄ and concentrated under reduced pressure to obtain crude, which was purified by silica gel column chromatography eluting with 50% EtOAc in PE to afford compound 1 (200 mg, 43%) as a yellow solid. LC-MS: m/z=201.0 [M+H]⁺

### (2-(dimethylamino)thiazol-4-yl)methanol (2)

To a stirring solution of compound 1 (200 mg, 1 mmol) in dry THF (10 mL) was added LiAlH₄ (2M in THF, 1 ml, 2 mmol) at 0° C. and stirred for 3 h. After consumption of the starting material (by TLC), the reaction mixture was diluted with water and extracted with EtOAc. Combined organic extracts were dried over anhydrous Na₂SO₄ and concentrated under reduced pressure to obtain crude, which was purified by silica gel column chromatography eluting with 50% EtOAc in PE to afford compound 2 (100 mg, 63%) as a yellow solid. LC-MS: m/z=159.0 [M+H]⁺

### 2-(dimethylamino)thiazole-4-carbaldehyde (3)

To a stirring solution of compound 2 (100 mg, 0.63 mmol) in dry DCM (10 mL) was added Dess-Martin (2 eq) at RT and stirred overnight. After consumption of the starting material (by TLC), the reaction mixture was diluted with water and extracted with DCM. Combined organic extracts were dried over anhydrous Na₂SO₄ and concentrated under reduced pressure to obtain crude, which was purified by silica gel column chromatography eluting with 50% EtOAc in PE to afford compound 3 (50 mg, 51%) as a yellow solid. LC-MS: m/z=157.0 [M+H]⁺

### (S)-4-((1-cyclopropylethylamino)methyl)-N,N-dimethylthiazol-2-amine (4)

To a stirring solution of compound 3 (50 mg, 0.32 mmol) and (S)-1-cyclopropylethanamine (41 mg, 0.48 mmol) in CH₃OH (10 mL) was added NaBH₄ (24 mg, 0.64 mmol) at RT and stirred overnight. After consumption of the starting material (by TLC), the reaction mixture was quenched with H₂O and extracted with EtOAc. Combined organic extracts were dried over anhydrous Na₂SO₄ and concentrated under reduced pressure to obtain crude compound 4 (50 mg, 69%) as an off-white solid, which was used for next step directly. LC-MS: m/z=226.1 [M+H]⁺

### N—((S)-1-cyclopropylethyl)-N-((2-(dimethylamino)thiazol-4-yl)methyl)-2-((S)-2,5-dioxo-2′,3′-dihydrospiro[imidazolidine-4,1′-indene]-1-yl)acetamide

To a stirring solution of compound 4 (50 mg, 0.22 mmol) and intermediate 9 (57 mg, 0.22 mmol) in DMF (1 mL) was added HATU (99 mg, 0.26 mmol) followed by DIPEA (38 mg, 0.29 mmol) and stirred at RT for 1 h. The reaction mixture was diluted with water and extracted with EtOAc. Combined organic extracts were dried over anhydrous Na₂SO₄ and concentrated under reduced pressure to obtain crude product, which was purified by Prep-HPLC, eluting with a gradient of CH₃CN/H₂O (containing 0.1% TFA) from 10/90 to 70/30 to give the desired product compound 255-1 (30 mg, 29%) as a white solid. LC-MS: m/z=468.0 [M+H]⁺ at RT 3.34 (97.9% purity). ¹H NMR (400 MHz, CD₃OD) δ 7.50-7.24 (m, 4H), 7.16-7.06 (m, 1H), 4.81-4.61 (m, 2H), 4.4-4.37 (m, 2H), 3.74-3.64 (m, 0.5H), 3.31-3.27 (m, 0.5H), 3.28-3.07 (m, 5H), 3.04 (s, 3H), 2.79-2.68 (m, 1H), 2.40-2.29 (m, 1H), 1.39-1.30 (m, 3H), 0.99-0.83 (m, 1H), 0.74-0.21 (m, 4H).

### Example 256

### 2H-spiro[benzo[b]thiophene-3,4′-imidazolidine]-2′,5′-dione,1,1-dioxide

To a stirring solution of SM1 (728 mg, 4 mmol) in 50% EtOH/H₂O (20 mL) was added (NH₄)₂CO₃ (3.07 g, 32 mmol) followed by potassium cyanide (780 mg, 12 mmol) at RT. The reaction mixture was heated to 75° C. for 18 h. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by silica gel column chromatography eluting with 10% MeOH in DCM to afford compound 1 (444 mg, 44%) as a white solid. LC-MS: m/z 253.0 [M+H]⁺

### N-benzyl-N—((R)-1-cyclopropyl-2,2,2-trifluoroethyl)-2-(2′,5′-dioxo-2H-spiro[benzo[b]thiophene, 1,1-dioxide-3,4′-imidazolidine]-1′-yl)acetamide

To a stirring solution of compound 1 (35 mg, 0.14 mmol) and intermediate 2 (49 mg, 0.14 mmol) in DMF (6 mL) was added K₂CO₃ (39 mg, 0.28 mmol) and stirred at RT overnight. After consumption of the starting material (by TLC), the reaction mixture was diluted with water and extracted with EtOAc. Combined organic extracts were dried over anhydrous Na₂SO₄ and concentrated under reduced pressure to obtain crude product, which was purified by Prep-HPLC, eluting with a gradient of CH₃CN/H₂O (containing 0.1% TFA) from 10/90 to 70/30 to give the desired product compound 256-1 (30 mg, 41%) as an off-white solid. LC-MS: m/z 522.0 [M+H]⁺ at RT 4.58 (98.20% purity). ¹H NMR (400 MHz, DMSO-d₆) δ 9.34 (dd, J=29.0, 4.4 Hz, 1H), 8.10-7.52 (m, 5H), 7.54-7.12 (m, 4H), 5.10-4.73 (m, 2H), 4.65-4.11 (m, 3H), 4.10-3.73 (m, 2H), 1.27-1.14 (m, 1H), 0.83-0.71 (m, 2H), 0.48-0.07 (m, 2H).

### Example 257

### tert-butyl 5-formyl-3-methoxypyridin-2-ylcarbamate (1)

To a stirring solution of (Boc)₂O (262 mg, 1.2 mmol) in CH₃OH (10 mL) was added the suspension of 6-amino-5-methoxynicotinaldehyde SM 1 (152 mg, 1 mmol) and Et₃N(202 mg, 2 mmol) in CH₃OH (20 mL) at RT and stirred overnight. The reaction mixture was concentrated and the residue was diluted with H₂O and extracted with EtOAc. Combined organic extracts were dried over anhydrous Na₂SO₄ and concentrated under reduced pressure to obtain crude compound 1 (214 mg, 85%) as a white solid. LC-MS: m/z=153.1 [M+H-BOC]⁺

### (S)-tert-butyl 5-((1-cyclopropylethylamino)methyl)-3-methoxypyridin-2-ylcarbamate (2)

To a stirring solution of compound 1 (28 mg, 0.18 mmol) and (S)-1-cyclopropylethanamine (30 mg, 0.36 mmol) in CH₃OH (10 mL) was added NaBH₄ (20 mg, 0.54 mmol) at RT and stirred for 3 h. After consumption of the starting material (by TLC), the reaction mixture was quenched with H₂O and extracted with EtOAc. Combined organic extracts were dried over anhydrous Na₂SO₄ and concentrated under reduced pressure to obtain crude compound 2 (53 mg, 92%) as an off-white solid, which was used for next step directly. LC-MS: m/z=322.2 [M+H]⁺

### tert-butyl 5-((N—((S)-1-cyclopropylethyl)-2-((S)-2,5-dioxo-2′,3′-dihydrospiro[imidazolidine-4,1′-indene]-1-yl)acetamido)methyl)-3-methoxypyridin-2-ylcarbamate (3)

To a stirring solution of compound 2 (50 mg, 0.15 mmol) and intermediate 9 (40 mg, 0.15 mmol) in DMF (1 mL) was added HATU (114 mg, 0.3 mmol) followed by DIPEA (39 mg, 0.3 mmol) and stirred at RT for 1 h. The reaction mixture was diluted with water and extracted with EtOAc. Combined organic extracts were dried over anhydrous Na₂SO₄ and concentrated under reduced pressure to afford crude compound 3 (34 mg, 40%) as a white solid, which was used for next step directly. LC-MS: m/z=464.2 [M+H-Boc]⁺

### N-((6-amino-5-methoxypyridin-3-yl)methyl)-N—((S)-1-cyclopropylethyl)-2-((S)-2,5-dioxo-2′,3′-dihydrospiro[imidazolidine-4,1′-indene]-1-yl)acetamide

To a stirring solution of compound 3 (34 mg, 0.06 mmol) in DCM (6 mL) was added TFA (2 mL). The reaction mixture was stirred at room temperature for 2 h. After consumption of the starting material (by TLC), the reaction mixture was concentrated under reduced pressure to obtain crude product, which was purified by Prep-HPLC, eluting with a gradient of CH₃CN/H₂O (containing 0.1% TFA) from 10/90 to 70/30 to give the desired product compound 257-1 (9 mg, 32%) as a white solid. LC-MS: m/z=464.2 [M+H]⁺ at RT 3.00 (95.4% purity). ¹H NMR (400 MHz, CD₃OD) δ 7.73-7.11 (m, 6H), 4.82-4.57 (m, 2H), 4.55-4.29 (m, 2H), 4.13-3.72 (m, 3.5H), 3.45-3.38 (m, 0.5H), 3.25-3.13 (m, 2H), 2.78-2.64 (m, 1H), 2.41-2.29 (m, 1H), 1.48-1.25 (m, 3H), 1.09-0.96 (m, 1H), 0.76-0.25 (m, 4H).

### Example 258

### Imidodicarbonic acid, (5-formyl-2-pyridinyl)-, bis(1,1-dimethylethyl) ester (1)

To a stirring solution of (Boc)₂O (1.75 g, 8 mmol) in DCM (10 mL) was added the suspension of 6-aminonicotinaldehyde SM 1 (488 mg, 4 mmol), DMAP (25 mg, 0.2 mmol) and Et₃N(1.2 g, 12 mmol) in DCM (20 mL) at RT and stirred overnight. The reaction mixture was concentrated and the residue was diluted with H₂O and extracted with DCM. Combined organic extracts were dried over anhydrous Na₂SO₄ and concentrated under reduced pressure to obtain crude product, which was purified by silica gel column chromatography eluting with 50% EtOAc in PE to afford compound 1 (1.2 g, 93%) as white solid. LC-MS: m/z=123.0 [M+H-BOC]⁺

### (S)-tert-butyl 5-((1-cyclopropylethylamino)methyl)pyridin-2-ylcarbamate (2)

To a stirring solution of compound 1 (600 mg, 1.86 mmol) and (S)-1-cyclopropylethanamine (238 mg, 2.8 mmol) in CH₃OH (10 mL) was added NaBH₃CN (586 mg, 9.3 mmol) at RT and stirred overnight. After consumption of the starting material (by TLC), the reaction mixture was quenched with H₂O and extracted with EtOAc. Combined organic extracts were dried over anhydrous Na₂SO₄ and concentrated under reduced pressure to obtain crude product, which was purified by silica gel column chromatography eluting with 10% CH₃OH in DCM to afford compound 2 (387 mg, 71%) as off-white solid. LC-MS: m/z=192.2 [M+H-BOC]⁺

### tert-butyl 5-((N—((S)-1-cyclopropylethyl)-2-((S)-2,5-dioxo-2′,3′-dihydrospiro[imidazolidine-4,1′-indene]-1-yl)acetamido)methyl)pyridin-2-ylcarbamate (3)

To a stirring solution of compound 2 (90 mg, 0.31 mmol) and intermediate 9 (60 mg, 0.23 mmol) in DMF (1 mL) was added HATU (99 mg, 0.26 mmol) followed by DIPEA (38 mg, 0.29 mmol) and stirred at RT for 1 h. The reaction mixture was diluted with water and extracted with EtOAc. Combined organic extracts were dried over anhydrous Na₂SO₄ and concentrated under reduced pressure to afford crude compound 3 (125 mg, 76%) as a white solid, which was used for next step directly. LC-MS: m/z=434.3 [M+H-BOC]⁺

### N-((6-aminopyridin-3-yl)methyl)-N—((S)-1-cyclopropylethyl)-2-((S)-2,5-dioxo-2′,3′-dihydrospiro[imidazolidine-4,1′-indene]-1-yl)acetamide

To a stirring solution of compound 3 (125 mg, 0.23 mmol) in DCM (6 mL) was added TFA (2 mL). The reaction mixture was stirred at room temperature for 2 h. After consumption of the starting material (by TLC), the reaction mixture was concentrated under reduced pressure to obtain crude product, which was purified by Prep-HPLC, eluting with a gradient of CH₃CN/H₂O (containing 0.1% TFA) from 10/90 to 70/30 to give the desired product compound 258-1 (13 mg, 13%) as a white solid. LC-MS: m/z=434.2 [M+H]⁺ at RT 4.68 (100% purity). ¹H NMR (400 MHz, CD₃OD) δ 8.06-7.87 (m, 1H), 7.76 (s, 1H), 7.41-7.28 (m, 4H), 7.09-6.91 (m, 1H), 4.82-4.65 (m, 2H), 4.56-4.35 (m, 2H), 3.86-3.81 (s, 0.5H), 3.44-3.36 (m, 0.5H), 3.22-3.08 (m, 2H), 2.78-2.68 (m, 1H), 2.41-2.29 (m, 1H), 1.42-1.28 (m, 3H), 1.07-1.01 (m, 1H), 0.79-0.19 (m, 4H).

### Example 259

### methyl 4-bromo-2-(methylsulfonyl)benzoate (1)

A solution of 4-bromo-2-(methylsulfonyl)benzoic acid SM 1 (2 g, 7.16 mmol) in MeOH (30 mL) was added H₂SO₄ (5 ml) stirred at reflux for 16 h. Quenched with water, extracted with EtOAc, washed with water and brine, concentrated under reduced pressure to obtain crude product, which was purified by combiflash (petroleum ether:ethyl acetate=1:1) to give compound 1 (2.0 g, 95%) as a white solid. LC-MS: m/z=293.1/295.1 [M+H]⁺

### 6-bromobenzo[b]thiophen-3 (2H)-one, 1,1-dioxide (2)

To a stirring solution of compound 1 (1.7 g, 5.8 mmol) in dry THF (20 mL) was added NaH (139 mg, 5.8 mmol), then stirred at RT for 2 h. Quenched with water and HCl (1N, 10 ml), extracted with EtOAc, concentrated under reduced pressure to obtain crude product, which was purified by combiflash (petroleum ether:ethyl acetate=1:1) to give compound 2 (1.4 g, 92%) as a yellow solid. LC-MS: m/z=261.0/263.0 [M+H]⁺

### 6-bromo-2H-spiro[benzo[b]thiophene-3,4′-imidazolidine]-2′,5′-dione,1,1-dioxide (3)

To a solution of compound 2 (0.52 g, 2 mmol) and in formamide (15 mL) was added (NH₄)₂CO₃ (1.15 g, 12 mmol) and KCN (390 mg, 6 mmol), then the reaction mixture was stirred at 120° C. for 16 h. Cool to RT, which was purified by Prep-HPLC, eluting with a gradient of CH₃CN/H₂O (containing 0.1% TFA) from 10/90 to 60/40 to give the desired compound 3 (270 mg, 41%) as a brown solid. LC-MS: m/z=331.1/333.1 [M+H]⁺

### N-benzyl-2-(6-bromo-2′,5′-dioxo-2H-spiro[benzo[b]thiophene, 1,1-dioxide-3,4′-imidazolidine]-1′-yl)-N—((S)-1-cyclopropylethyl)acetamide

A solution of compound 4 (270 mg, 0.81 mmol) and intermediate 23 (263 mg, 0.89 mmol) in DMF (10 mL) was added K₂CO₃ (335 mg, 2.43 mmol). The reaction mixture was stirred at room temperature for 2 h. Quenched with water and extracted with EtOAc, dried and concentrated under reduced pressure to obtain crude product, which was purified by Prep-HPLC, eluting with a gradient of CH₃CN/H₂O (containing 0.1% TFA) from 10/90 to 70/30 to give the desired product compound 259-1 (95 mg, 21%) as a white solid. LC-MS: m/z=546.0/548.0 [M+H]⁺ at RT 5.03 (98.8% purity). ¹H NMR (400 MHz, CD₃OD) δ 8.08-8.01 (m, 1H), 8.00-7.92 (m, 1H), 7.87-7.80 (m, 1H), 7.52-7.18 (m, 5H), 4.84-4.34 (m, 4H), 4.16-4.02 (m, 1H), 3.91-3.77 (m, 1H), 3.41-3.35 (m, 1H), 1.39-1.19 (m, 3H), 1.06-0.90 (m, 1H), 0.68-0.52 (m, 1H), 0.44-0.20 (m, 3H).

### Example 260

### N-benzyl-N—((S)-1-cyclopropylethyl)-2-(2′,5′-dioxo-6-(1H-pyrazol-3-yl)-2H-spiro[benzo[b]thiophene, 1,1-dioxide-3,4′-imidazolidine]-1′-yl)acetamide

A solution of compound 268-1 (32 mg, 0.06 mmol) and 1H-pyrazol-3-ylboronic acid (20 mg, 0.18 mmol) in dioxane (10 mL) and water (1 mL) was added NaHCO₃ (25 mg, 0.3 mmol) and Pd(dppf)Cl₂ (4 mg, 0.006 mmol). The reaction mixture was stirred at 100° C. for 16 h under N₂. Quenched with water and extracted with EtOAc, dried and concentrated under reduced pressure to obtain crude product, which was purified by Prep-HPLC, eluting with a gradient of CH₃CN/H₂O (containing 0.1% TFA) from 10/90 to 70/30 to give the desired product compound 260-1 (20 mg, 62%) as a white solid. LC-MS: m/z=534.0 [M+H]⁺ at RT 4.12 (98.4% purity). ¹H NMR (301 MHz, CD₃OD) δ 8.25-8.09 (m, 1H), 7.92-7.81 (m, 1H), 7.77-7.65 (m, 1H), 7.56-7.06 (m, 5H), 6.87-6.76 (m, 1H), 4.80-4.32 (m, 4H), 4.14-4.00 (m, 1H), 3.89-3.74 (m, 1H), 3.41-3.31 (m, 1H), 1.34-1.09 (m, 3H), 1.02-0.79 (m, 1H), 0.64-0.47 (m, 1H), 0.42-0.01 (m, 3H).

### Example 261

tert-butyl 1-(2′,4′-dioxo-2,3-dihydrospiro[indene-1,5′-ozazolidine]-5-ylamino)-2-methyl-1-oxopropan-2-ylcarbamate (1):

A solution of intermediate 39 (0.35 g, 1.6 mmol) and 2-(tert-butoxycarbonylamino)-2-methylpropanoic acid (650 mg, 3.2 mmol) in DCM (20 mL) was added TEA (808 mg, 8 mmol) and T₃P (1.01 g, 3.2 mmol), then stirred at RT for 16 h. Quenched with water, extracted with DCM, dried and concentrated under reduced pressure to obtain crude product, which was purified by combiflash (methanol:DCM=1:20) to give compound 1 (90 mg, 14%). LC-MS: m/z 304.1 [M+H-Boc]⁺

### tert-butyl 1-((S)-3′-(2-((4-bromobenzyl)((S)-1-cyclopropylethyl)amino)-2-oxoethyl)-2′,4′-dioxo-2,3-dihydrospiro[indene-1,5′-ozazolidine]-5-ylamino)-2-methyl-1-oxopropan-2-ylcarbamate (2)

To a stirring solution of compound 1 (85 mg, 0.2 mmol) and intermediate 51 (90 mg, 0.24 mmol) in dry DMF (5 mL) was added K₂CO₃ (82 mg, 0.6 mmol), then stirred at RT for 2 h. Quenched with water, extracted with EtOAc, concentrated under reduced pressure to obtain crude product, which was purified by combiflash (methanol:DCM=1:20) to give compound 2 (0.12 g, 86%). LC-MS: m/z=598.0/600.0 [M+H-Boc]⁺

### 2-amino-N—((S)-3′-(2-((4-bromobenzyl)((S)-1-cyclopropylethyl)amino)-2-oxoethyl)-2′,4′-dioxo-2,3-dihydrospiro[indene-1,5′-ozazolidine]-5-yl)-2-methylpropanamide: and 2-amino-N—((R)-3′-(2-((4-bromobenzyl)((S)-1-cyclopropylethyl)amino)-2-oxoethyl)-2′,4′-dioxo-2,3-dihydrospiro[indene-1,5′-ozazolidine]-5-yl)-2-methylpropanamide

A solution of compound 2 (120 mg, 0.18 mmol) in DCM (10 mL) was added TFA (1 mL). The reaction mixture was stirred at room temperature for 1 h. Concentrated under reduced pressure to obtain crude product, which was purified by Chiral-HPLC (column: CHIRALPAK IB; Manufacturer: DAICEL CHIRAL TECHNOLOGIES (CHINA) CO., LTD; condition: EtOH/Hexane (1:1, 0.2% of FA and 0.2% Diethylamine in EtOH)) to give compound 261-1 (41 mg, 38%) as a white solid and compound 261-2 (40 mg, 38%) as a white solid. LC-MS: m/z=597.2/599.1 [M+H]⁺ at RT 15.4 (100% purity). ¹H NMR (400 MHz, CD₃OD) δ 7.76 (s, 1H), 7.59-7.22 (m, 6H), 4.92-4.42 (m, 4H), 3.90-3.80 (m, 1H), 3.32-3.18 (m, 2H), 2.89 2-50 (m, 2H), 1.51-1.20 (m, 9H), 1.01-0.89 (m, 1H), 0.68-059 (m, 1H), 0.42-0.25 (m, 3H). LC-MS: m/z=597.2/599.1 [M+H]⁺ at RT 28.55 (97.8% purity). ¹H NMR (400 MHz, CD₃OD) δ 7.77 (s, 1H), 7.62-7.18 (m, 6H), 4.95-4.40 (m, 4H), 3.90-3.80 (m, 1H), 3.32-3.18 (m, 2H), 2.89 2-50 (m, 2H), 1.51-1.20 (m, 9H), 1.01-0.89 (m, 1H), 0.68-059 (m, 1H), 0.42-0.25 (m, 3H).

### Example 262

### methyl 2-(((benzyloxy)carbonyl)amino)-2-(oxetan-3-ylidene)acetate (1)

To a stirring solution of SM 1 (720 mg, 10 mmol) and tetramethylguanidine (1.15 g, 10 mmol) in THF (20 mL) was added 2-(((benzyloxy)carbonyl)amino)propanoic (dimethyl phosphoric) anhydride (3.44 g, 10.4 mmol) at −70° C. The resulting mixture was warmed to RT and stirred overnight. The reaction mixture was quenched with H₂O and extracted with EtOAc. Combined organic extracts were dried over anhydrous Na₂SO₄ and concentrated under reduced pressure to obtain crude product, which was purified by silica gel column chromatography eluting with 5% CH₃OH in DCM to afford compound 1 (2.2 g, 79%) as a solid. LC-MS: m/z=278.1 [M+H]⁺

### methyl 2-amino-2-(oxetan-3-yl)acetate (2)

To a stirring solution of compound 1 (2.2 g, 7.9 mmol) in DCM:MeOH=1:1 (V/V, 30 mL) was added PdOH/C (0.1 eq) at RT and stirred overnight. Filtered and the filtrate was concentrated under reduced pressure to obtain crude product, which was purified by silica gel column chromatography eluting with 5% CH₃OH in DCM to afford compound 2 (640 mg, 56%) as a solid. LC-MS: m/z=146.0 [M+H]⁺

### (R)-methyl 2-amino-2-(oxetan-3-yl)acetate (3)

To a stirring solution of compound 2 (320 mg, 2.2 mmol) and in EtOH (5 mL) was added (2R,3R)-2,3-dihydroxysuccinic acid (330 mg, 2.2 mmol) followed by benzaldehyde (23 mg, 0.22 mmol) at RT and stirred overnight. The reaction mixture was quenched with H₂O and extracted with EtOAc. Combined organic extracts were dried over anhydrous Na₂SO₄ and concentrated under reduced pressure to obtain crude product, which was purified by silica gel column chromatography eluting with 5% CH₃OH in DCM to afford compound 3 (100 mg, 31%) as a solid. LC-MS: m/z 10=146.0 [M+H]⁺

### (R)-methyl 2-((tert-butoxycarbonyl)amino)-2-(oxetan-3-yl)acetate (4)

To a stirring solution of compound 3 (100 mg, 0.69 mmol) and in THF (10 mL) was added (Boc)₂O (180 mg, 0.83 mmol) followed by Et₃N (140 mg, 1.38 mmol) at RT and stirred overnight. The reaction mixture was diluted with H₂O and extracted with EtOAc. Combined organic extracts were dried over anhydrous Na₂SO₄ and concentrated under reduced pressure to obtain crude product, which was purified by silica gel column chromatography eluting with 5% CH₃OH in DCM to afford compound 4 (100 mg, 59%) as a solid. LC-MS: m/z=146.0 [M+H-Boc]⁺

### (R)-2-((tert-butoxycarbonyl)amino)-2-(oxetan-3-yl)acetic acid (5)

To a stirring solution of compound 4 (100 mg, 0.69 mmol) and in CH₃OH (5 mL) was added 1N NaOH (1 mL) at RT and stirred overnight. The reaction mixture was concentrated under reduced pressure to remove solvent. The residue was diluted with H₂O and EtOAc. The water layer was adjusted the pH to 3-4 with 6N HCl_{aq}. and extracted with EtOAc. Combined organic extracts were dried over anhydrous Na₂SO₄ and concentrated under reduced pressure to afford crude compound 5 (130 mg, 82%) as a solid, which was used for next step directly. LC-MS: m/z=132.1 [M+H-Boc]⁺

### tert-butyl ((1R)-2-((3′-(2-(benzyl((S)-1-cyclopropylethyl)amino)-2-oxoethyl)-2′,4′-dioxo-2,3-dihydrospiro[indene-1,5′-ozazolidin]-5-yl)amino)-1-(oxetan-3-yl)-2-oxoethyl)carbamate (6)

To a stirring solution of intermediate 18 (95 mg, 0.22 mmol) and compound 5 (50 mg, 0.22 mmol) in DCM (20 mL) was added Et₃N (45 mg, 0.44 mmol) followed by T₃P (50% in EtOAc) (280 mg, 0.44 mmol) and stirred at RT for 1 h. The reaction mixture was diluted with water and extracted with DCM. Combined organic extracts were dried over anhydrous Na₂SO₄ and concentrated under reduced pressure to obtain crude product, which was purified by silica gel column chromatography eluting with 5% MeOH in DCM to afford compound 6 (92 mg, 65%) as a solid. LC-MS: m/z=547.3 [M+H-Boc]⁺

### (2R)-2-amino-N-(3′-(2-(benzyl((S)-1-cyclopropylethyl)amino)-2-oxoethyl)-2′,4′-dioxo-2,3-dihydrospiro[indene-1,5′-ozazolidin]-5-yl)-2-(oxetan-3-yl)acetamide

To a stirring solution of compound 6 (20 mg, 0.03 mmol) in DCM (5 mL) was added ZnBr₂ (7 mg, 0.03 mmol) at RT and stirred for 2 h. The mixture was concentrated under reduced pressure to obtain crude product, which was purified by Prep-HPLC, eluting with a gradient of CH₃CN/H₂O (containing 0.1% TFA) from 10/90 to 70/30 to give the desired product compound 262-1 (10 mg, 61%) as a white solid. LC-MS: m/z=547.1 [M+H]⁺ at RT 3.85 (93.08% purity). ¹H NMR (400 MHz, CD₃OD) δ 7.75 (s, 1H), 7.52-7.20 (m, 7H), 4.86-4.76 (m, 4H), 4.73-4.64 (m, 2H), 4.64-4.39 (m, 2H), 3.90-3.79 (m, 1.5H), 3.41-3.35 (m, 1.5H), 3.30-3.05 (m, 2H), 2.84-2.28 (m, 2H), 1.33-1.21 (m, 3H), 1.02-0.94 (m, 1H), 0.67-0.27 (m, 4H).

### Example 263

### 1,3-Dibromo-2,2-dimethoxypropane (1)

To a stirring solution of propan-2-one SM 1 (11.6 g, 0.2 mol) in CH₃OH(50 mL) was added dibromine (64 g, 0.4 mol) and stirred at RT overnight. The reaction mixture was concentrated under reduced pressure. The residue was diluted with water and extracted with EtOAc. Combined organic extracts were dried over anhydrous Na₂SO₄ and concentrated under reduced pressure to obtain crude compound 1 (34 g, 65%) as a solid, which was used for next step directly.

### Diisopropyl 3,3-dimethoxycyclobutane-1,1-dicarboxylate (2)

To a stirring suspension of NaH (11.4 g, 0.28 mol) in DMF (100 mL) was added diisopropyl malonate (48.9 g, 0.26 mol) in a drop manner under N₂ (g) keeping the temperature below 70° C. After 2 hours compound 1 (34 g, 0.13 mol) was added in one portion and the mixture was heated 140° C. overnight. After consumption of the starting material (by TLC), the reaction mixture was poured into saturated NH₄Cl_{aq} and extracted with EtOAc. Combined organic extracts were dried over anhydrous Na₂SO₄ and concentrated under reduced pressure to obtain crude product, which was purified by silica gel column chromatography eluting with 20% EtOAc in Hexane to afford compound 2 (5.9 g, 16%) as a yellow oil.

### Diisopropyl 3-oxocyclobutane-1,1-dicarboxylate (3)

The suspension of compound 2 (5.9 g, 20.5 mmol) in 6N HCl_{aq} (60 mL) was stirred at RT overnight. After consumption of the starting material (by TLC), the reaction mixture was extracted with EtOAc. Combined organic extracts were dried over anhydrous Na₂SO₄ and concentrated under reduced pressure to obtain crude compound 3 (4.9 g, 100%) as a yellow oil.

### Diisopropyl 3,3-difluorocyclobutane-1,1-dicarboxylate (4)

To a stirring suspension of compound 3 (4.9 g, 20 mmol) in DCM (50 mL) was added morpholin-4-ylsulphur trifluoride (7.7 g, 44 mol) at 0° C. slowly. The resulting reaction mixture was warmed to RT for 3 days. The reaction mixture was poured into saturated NaHCO_{3aq} and extracted with DCM. Combined organic extracts were dried over anhydrous Na₂SO₄ and concentrated under reduced pressure to afford crude product, which was purified by silica gel column chromatography eluting with 10% MeOH in DCM to afford compound 4 (4.2 g, 79%) as a yellow oil.

### 3,3-Difluoro-1-(isopropoxycarbonyl)cyclobutanecarboxylic acid (5)

To a stirring solution of compound 4 (4.2 g, 15.9 mmol) in CH₃OH(20 mL) was added a solution of NaOH (636 mg) in H₂O (10 mL) at 0° C. The resulting reaction mixture was warmed to RT and stirred overnight. The reaction mixture was concentrated under reduced pressure to remove solvent. The residue was diluted with H₂O and EtOAc. The water layer was adjusted the pH to 4 with 6N HCl_{aq}. and extracted with EtOAc. Combined organic extracts were dried over anhydrous Na₂SO₄ and concentrated under reduced pressure to afford crude compound 5 (1.3 g, 37%) as a yellow oil, which was used for next step directly.

### 1-Amino-3,3-difluorocyclobutanecarboxylic acid (6)

To a stirring solution of compound 5 (600 mg, 2.7 mmol) in DCM (10 mL) was added SOCl₂ (0.6 mL, 8.1 mmol) and heated to reflux for 3 h. The reaction mixture was concentrated under reduced pressure to remove solvent. The residue was dissolved in acetone (10 mL) and cooled to 0° C. To the obtained mixture was added a solution of NaN₃ (352 mg) in H₂O (10 mL) and stirred at 0° C. for next 2 h. Acetone was gently removed at reduced pressure and toluene (20 mL) was added. The organic layer was dried over anhydrous Na₂SO₄ and was refluxed for 2 hours. Then the reaction mixture was concentrated, treated with 3N HCl_{aq}. and refluxed for 6 hours. Extracted with EtOAc and the water layer was concentrated under reduced pressure to afford crude compound 6 (180 mg, 35%) as an off-white solid.

### Sodium 1-(tert-butoxycarbonylamino)-3,3-difluorocyclobutanecarboxylate (7)

To a stirring solution of compound 6 (130 mg, 0.86 mmol) in THF/H₂O (3 mL/3 mL) was added NaHCO₃ (144 mg, 1.72 mmol) followed by (BoC)₂O (188 mg, 0.86 mmol) in THF (1 mL). The resulting mixture was stirred at 80° C. overnight. Extracted with DCM and the water layer was concentrated under reduced pressure to afford crude compound 7 (110 mg, 47%) as a yellow solid.

### tert-butyl 1-(3′-(2-(benzyl((S)-1-cyclopropylethyl)amino)-2-oxoethyl)-2′,4′-dioxo-2,3-dihydrospiro[indene-1,5′-ozazolidine]-5-ylcarbamoyl)-3,3-difluorocyclobutylcarbamate (8)

To a stirring solution of intermediate 18 (173 mg, 0.4 mmol) and compound 7 (110 mg, 0.4 mmol) in DCM (20 mL) was added Et₃N (81 mg, 0.8 mmol) followed by T₃P (50% in EtOAc) (509 mg, 0.8 mmol) and stirred at RT for 1 h. The reaction mixture was diluted with water and extracted with DCM. Combined organic extracts were dried over anhydrous Na₂SO₄ and concentrated under reduced pressure to obtain crude product, which was purified by silica gel column chromatography eluting with 5% MeOH in DCM to afford compound 8 (105 mg, 39%) as a light yellow solid. LC-MS: m/z=667.2 [M+H]⁺

### 1-Amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)-3,3-difluorocyclobutanecarboxamide

To a stirring solution of compound 8 (105 mg, 0.16 mmol) in DCM (10 mL) was added TFA (2 mL). The reaction mixture was stirred at room temperature for 2 h. After consumption of the starting material (by TLC), the reaction mixture was concentrated under reduced pressure to obtain crude product, which was purified by Prep-HPLC, eluting with a gradient of CH₃CN/H₂O (containing 0.1% TFA) from 10/90 to 70/30 to give the desired product compound 263-1 (37 mg, 34%) as a TFA salt. LC-MS: m/z=567.2 [M+H]⁺ at RT 4.10 (92% purity). ¹H NMR (400 MHz, DMSO-d₆) δ 10.18 (s, 1H), 7.75 (s, 1H), 7.60-7.13 (m, 7H), 4.80 (s, 1H), 4.74-4.60 (m, 1H), 4.56-4.32 (m, 2H), 3.83-2.72 (m, 0.5H), 3.52-3.42 (m, 2.5H), 3.27-2.98 (m, 4H), 2.79-2.52 (m, 4H), 1.28-1.11 (m, 3H), 1.03-0.94 (m, 1H), 0.57-0.12 (m, 4H).

### Example 264

### 2-(tert-butoxycarbonylamino)-3,3,3-trifluoropropanoic acid (1)

A solution of 2-amino-3,3,3-trifluoropropanoic acid SM 1 (3 g, 21 mmol) and (Boc)₂O (9.16 g, 42 mmol) in MeOH (100 mL) and TEA (10 mL) was NaOH (21 mL, 1N in water), stirred at RT for 16 h. Quenched with water, extracted with EtOAc, then the water layer was adjusted PH to 2˜3 with HCl (2N), extracted with EtOAc and dried, concentrated under reduced pressure to give compound 1 (3.7 g, 73%). LC-MS: m/z=144.1 [M+H-Boc]⁺

### tert-butyl 3-(3′-(2-(benzyl((S)-1-cyclopropylethyl)amino)-2-oxoethyl)-2′,4′-dioxo-2,3-dihydrospiro[indene-1,5′-ozazolidine]-5-ylamino)-1,1,1-trifluoro-3-oxopropan-2-ylcarbamate (2)

To a solution of intermediate 18 (433 mg, 1 mmol) and compound 1 (243 mg, 1 mmol) in DCM (10 mL) was added TEA (303 mg, 3 mmol) and T₃P (636 mg, 2 mmol), then the reaction mixture was stirred at RT for 2 h. Quenched with water, extracted with DCM, washed with water and brine, dried and concentrated under reduced pressure to obtain crude product, which was purified by combiflash (methanol:DCM=1:20) to give compound 2 (610 mg, 92%) as a white solid. LC-MS: m/z=659.2 [M+H]⁺

### 2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)-3,3,3-trifluoropropanamide

A solution of compound 2 (6.04 g, 9.2 mmol) in DCM (100 mL) was added TFA (10 mL). The reaction mixture was stirred at room temperature for 2 h. Concentrated under reduced pressure to obtain crude product, which was purified by combiflash (methanol:DCM=1:20) to give compound 264-1 (4.57 g, 89%) as a white solid. LC-MS: m/z=559.0 [M+H]⁺ at RT 4.40 (100% purity). ¹H NMR (300 MHz, CD₃OD) δ 7.82-7.70 (m, 1H), 7.55-7.02 (m, 6H), 4.83-4.32 (m, 4H), 4.22-4.00 (m, 1H), 3.90-3.73 (m, 1H), 3.26-3.02 (m, 2H), 2.86-2.48 (m, 2H), 1.37-1.15 (m, 3H), 1.05-0.86 (m, 1H), 0.70-0.51 (m, 1H), 0.45-0.21 (m, 3H).

### Example 265

### (R)-2-amino-N—((S)-3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)-3-methylbutanamide

Compound 234-1 (500 mg) was purified by Chiral HPLC (column: CHIRAL IB; Manufacturer: DAICEL CHIRAL TECHNOLOGIES (CHINA) CO., LTD; Isopropyl alcohol/hexane (1; 1, 0.2% FA and 0.2% Diethylamine in isopropyl alcohol)) and Prep-HPLC, eluting with a gradient of CH₃CN/H₂O (containing 0.1% TFA) from 10/90 to 70/30 to give the desired compound 265-1 (168 mg, 38%) and compound 262-2 (153 mg, 31%) as TFA salt. LC-MS: m/z=551.2 [M+H]⁺ at RT 10.89 (100% purity). ¹H NMR (400 MHz, DMSO-d₆) δ 10.62 (s, 1H), 8.23 (s, 2H), 7.72 (s, 1H), 7.52-7.43 (m, 3H), 7.34-7.21 (m, 2H), 7.12 (t, J=8.9 Hz, 1H), 4.78 (s, 1H), 4.65 (t, J=8.4 Hz, 1H), 4.52-4.23 (m, 2H), 3.81-3.71 (s, 1.5H), 3.33-3.30 (m, 0.5H), 3.24-3.04 (m, 2H), 2.73-2.52 (m, 2H), 2.23-2.17 (m, 1H), 1.18 (dd, J=28.1, 6.6 Hz, 3H), 1.00 (t, J=6.2 Hz, 6H), 0.55-0.46 (m, 1H), 0.43-0.12 (m, 4H).

### (R)-2-amino-N—((R)-3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)-3-methylbutanamide

LC-MS: m/z=551.2 [M+H]⁺ at RT 13.89 (97% purity). ¹H NMR (400 MHz, DMSO-d₆) δ 10.65 (s, 1H), 8.25 (s, 2H), 7.74 (d, J=6.6 Hz, 1H), 7.45 (dd, J=12.9, 7.3 Hz, 3H), 7.34-7.19 (m, 2H), 7.12 (t, J=8.9 Hz, 1H), 4.78 (s, 1H), 4.68-4.59 (m, 1H), 4.54-4.27 (m, 2H), 3.81-3.70 (m, 1.5H), 3.42-3.37 (s, 0.5H), 3.25-3.02 (m, 2H), 2.75-2.53 (m, 2H), 2.25-2.13 (m, 1H), 1.19 (dd, J=34.7, 6.7 Hz, 3H), 1.05-0.91 (m, 6H), 0.56-0.45 (m, 1H), 0.42-0.13 (m, 4H).

### Example 266

### N-(4-fluorobenzyl)-2-((S)-5-(3-methylureido)-2′,4′-dioxo-2,3-dihydrospiro[indene-1,5′-ozazolidine]-3′-yl)-N—((S)-1,1,1-trifluoropropan-2-yl)acetamide: and N-(4-fluorobenzyl)-2-((R)-5-(3-methylureido)-2′,4′-dioxo-2,3-dihydrospiro[indene-1,5′-ozazolidine]-3′-yl)-N—((S)-1,1,1-trifluoropropan-2-yl)acetamide

Compound 228-1 (2.5 g, 4.7 mmol) was purified by SFC (column: CHIRALPAK AS-H; Manufacturer: DAICEL CHIRAL TECHNOLOGIES (CHINA) CO., LTD; condition: Isopropyl alcohol/CO₂ (3:7)) to give compound 266-1 (1.01 g, 40%) as a white solid and compound 266-2 (1.02 g, 40%) as a white solid. LC-MS: m/z=537.1 [M+H]⁺ at RT 3.79 (100% purity). ¹H NMR (301 MHz, CD₃OD) δ 7.56-7.47 (m, 1H), 7.44-7.30 (m, 2H), 7.26-7.10 (m, 3H), 7.08-6.96 (m, 1H), 5.53-5.35 (m, 1H), 4.89-4.24 (m, 5H), 3.26-2.98 (m, 2H), 2.85-2.67 (m, 4H), 2.63-2.42 (m, 1H), 1.50-1.29 (m, 3H). LC-MS: m/z=537.1 [M+H]⁺ at RT 5.15 (100% purity). ¹H NMR (301 MHz, CD₃OD) δ 7.56-7.48 (m, 1H), 7.43-7.30 (m, 2H), 7.27-7.08 (m, 3H), 7.00 (q, J=8.7 Hz, 1H), 5.49-5.36 (m, 1H), 4.86-4.22 (m, 5H), 3.25-2.97 (m, 2H), 2.89-2.66 (m, 4H), 2.62-2.45 (m, 1H), 1.51-1.33 (m, 3H).

### Example 267

### N—((S)-1-cyclopropylethyl)-N-(4-fluorobenzyl)-2-((S)-5-(3-methylureido)-2′,4′-dioxo-2,3-dihydrospiro[indene-1,5′-ozazolidine]-3′-yl)acetamide: and N—((S)-1-cyclopropylethyl)-N-(4-fluorobenzyl)-2-((R)-5-(3-methylureido)-2′,4′-dioxo-2,3-dihydrospiro[indene-1,5′-ozazolidine]-3′-yl)acetamide

A solution of intermediate 40 (600 mg, 1.33 mmol) in THF (20 mL) was added TEA (800 mg, 7.98 mmol) and triphosgen (790 mg, 2.66 mmol) at 0° C. under N₂. The reaction mixture was stirred at room temperature for 0.5 h. Then MeNH₂ (6.7 mL, 13.3 mmol) was added and stirred at RT for 1 h. Quenched with water and extracted with EtOAc, dried and concentrated under reduced pressure to obtain crude product, which was purified by combiflash to give off-white solid, then purified by SFC (column: CHIRALPAK OD-H; Manufacturer: DAICEL CHIRAL TECHNOLOGIES (CHINA) CO., LTD; condition: MeOH/CO₂ (3:7)) to give 267-1 (0.2 g, 33%) as a white solid and 267-2 (0.2 g, 33%) as a white solid. LC-MS: m/z=509.2 [M+H]⁺ at RT 4.44 (98.5% purity). ¹H NMR (400 MHz, CD₃OD) δ 7.60-6.93 (m, 7H), 4.81-4.35 (m, 4H), 3.92-3.77 (m, 1H), 3.24-3.03 (m, 2H), 2.86-2.68 (m, 4H), 2.62-2.47 (m, 1H), 1.43-1.20 (m, 3H), 1.04-0.87 (m, 1H), 0.68-0.53 (m, 1H), 0.47-0.22 (m, 3H). LC-MS: m/z=509.2 [M+H]⁺ at RT 5.63 (98.5% purity). ¹H NMR (400 MHz, CD₃OD) δ 7.60-6.93 (m, 7H), 4.81-4.32 (m, 4H), 3.89-3.75 (m, 1H), 3.26-3.01 (m, 2H), 2.89-2.68 (m, 4H), 2.63-2.45 (m, 1H), 1.38-1.18 (m, 3H), 1.03-0.86 (m, 1H), 0.69-0.52 (m, 1H), 0.47-0.19 (m, 3H).

### Example 268

### (S)-2-amino-N—((S)-3′-(2-(benzyl((S)-1-cyclopropylethyl)amino)-2-oxoethyl)-2′,4′-dioxo-2,3-dihydrospiro[indene-1,5′-ozazolidine]-5-yl)-3,3,3-trifluoropropanamide: (R)-2-amino-N—((S)-3′-(2-(benzyl((S)-1-cyclopropylethyl)amino)-2-oxoethyl)-2′,4′-dioxo-2,3-dihydrospiro[indene-1,5′-ozazolidine]-5-yl)-3,3,3-trifluoropropanamide: (S)-2-amino-N—((R)-3′-(2-(benzyl((S)-1-cyclopropylethyl)amino)-2-oxoethyl)-2′,4′-dioxo-2,3-dihydrospiro[indene-1,5′-ozazolidine]-5-yl)-3,3,3-trifluoropropanamide: and (R)-2-amino-N—((R)-3′-(2-(benzyl((S)-1-cyclopropylethyl)amino)-2-oxoethyl)-2′,4′-dioxo-2,3-dihydrospiro[indene-1,5′-ozazolidine]-5-yl)-3,3,3-trifluoropropanamide

264-1 was separated by Chiral-HPLC (column: CHIRALPAK IB; Manufacturer: DAICEL CHIRAL TECHNOLOGIES (CHINA) CO., LTD; condition: EtOH/Hexane (1:1, 0.2% of FA and 0.2% diethylamine in EtOH)) to give 268-1 (30 mg, 18%), 268-2 (0.15 mg, 9%), 268-3 (30 mg, 18%) and 268-4 (0.15 mg, 9%) as white solid. LC-MS: m/z=559.2 [M+H]⁺ at RT 7.51 (96.1% purity). ¹H NMR (400 MHz, CD₃OD) δ 7.84-7.71 (m, 1H), 7.60-7.37 (m, H), 7.35-7.18 (m, 3H), 4.85-4.31 (m, 5H), 3.93-3.81 (m, 1H), 3.30-3.05 (m, 2H), 2.88-2.75 (m, 1H), 2.67-2.52 (m, 1H), 1.39-1.23 (m, 3H), 1.06-0.87 (m, 1H), 0.71-0.52 (m, 1H), 0.44-0.23 (m, 3H). LC-MS: m/z=559.2 [M+H]⁺ at RT 8.00 (97.8% purity). ¹H NMR (300 MHz, CD₃OD) δ 7.83-7.71 (m, 1H), 7.60-7.09 (m, 7H), 4.87-4.31 (m, 5H), 3.89-3.77 (m, 1H), 3.25-3.04 (m, 2H), 2.87-2.68 (m, 1H), 2.64-2.47 (m, 1H), 1.32-1.18 (m, 3H), 1.03-0.84 (m, 1H), 0.66-0.44 (m, 1H), 0.40-0.16 (m, 3H). LC-MS: m/z=559.2 [M+H]⁺ at RT 10.99 (95.4% purity). ¹H NMR (400 MHz, CD₃OD) δ 7.85-7.73 (m, 1H), 7.66-7.12 (m, 7H), 4.82-4.38 (m, 5H), 3.91-3.79 (m, 1H), 3.28-3.06 (m, 2H), 2.86-2.75 (m, 1H), 2.70-2.54 (m, 1H), 1.36-1.18 (m, 3H), 1.05-0.87 (m, 1H), 0.70-0.51 (m, 1H), 0.41-0.11 (m, 3H). LC-MS: m/z=559.2 [M+H]⁺ at RT 17.01 (94.6% purity). ¹H NMR (400 MHz, CD₃OD) δ 7.84-7.75 (m, 1H), 7.70-7.07 (m, 7H), 4.83-4.37 (m, 5H), 3.92-3.80 (m, 1H), 3.28-3.09 (m, 2H), 2.89-2.75 (m, 1H), 2.67-2.53 (m, 1H), 1.36-1.21 (m, 3H), 1.04-0.89 (m, 1H), 0.68-0.52 (m, 1H), 0.45-0.20 (m, 3H).

### Example 269

### N-benzyl-N—((R)-1-cyclopropyl-2,2,2-trifluoroethyl)-2-((S)-5-(3-methylureido)-2′,4′-dioxo-2,3-dihydrospiro[indene-1,5′-ozazolidine]-3′-yl)acetamide

Compound 224-1 (3.55 g) was purified by SFC (column: CHIRAL CELL OD-H; Manufacturer: DAICEL CHIRAL TECHNOLOGIES (CHINA) CO., LTD; condition: EtOH/CO₂ (3:7, 0.2% FA and 0.2% Diethylamine in EtOH)) to afford compound 269-1 (1.3 g) compound 269-2 (1.3 g) as white solid. LC-MS: m/z=545.2 [M+H]⁺ at RT 7.79 (99.61% purity). ¹H NMR (400 MHz, DMSO-d₆) δ 8.71 (s, 1H), 7.55 (s, 1H), 7.42 (d, J=4.5 Hz, 3H), 7.36-7.17 (m, 4H), 6.09 (d, J=4.6 Hz, 1H), 5.11-4.82 (m, 2H), 4.61-4.52 (m, 2H), 4.37-4.22 (m, 1H), 3.16-2.93 (m, 2H), 2.67-2.58 (m, 4H), 2.49-2.42 (m, 1H), 1.24 (s, 1H), 0.79-0.66 (m, 2H), 0.40-0.16 (m, 2H).

### N-benzyl-N—((R)-1-cyclopropyl-2,2,2-trifluoroethyl)-2-((R)-5-(3-methylureido)-2′,4′-dioxo-2,3-dihydrospiro[indene-1,5′-ozazolidine]-3′-yl)acetamide

LC-MS: m/z=545.2 [M+H]⁺ at RT 16.82 (99.1% purity). ¹H NMR (400 MHz, DMSO-d₆) δ 8.71 (s, 1H), 7.56 (d, J=11.9 Hz, 1H), 7.42 (d, J=4.4 Hz, 3H), 7.34-7.19 (m, 4H), 6.09 (d, J=4.6 Hz, 1H), 5.13-4.88 (m, 2H), 4.68-4.25 (m, 3H), 3.18-2.91 (m, 2H), 2.69-2.57 (m, 4H), 2.50-2.42 (m, 1H), 1.24 (s, 1H), 0.83-0.72 (m, 2H), 0.47-0.13 (m, 2H).

### Example 270

### tert-butyl ((2R)-1-((3′-(2-((4-fluorobenzyl)((S)-1,1,1-trifluoropropan-2-yl)amino)-2-oxoethyl)-2′,4′-dioxo-2,3-dihydro spiro[indene-1,5′-ozazolidin]-5-yl)amino)-3-methyl-1-oxobutan-2-yl)carbamate (1)

To a stirring solution of intermediate 43 (200 mg, 0.48 mmol) and intermediate 46 (164 mg, 0.48 mmol) in DMF (10 mL) was added K₂CO₃ (99 mg, 0.72 mmol) and stirred at RT for 1 h. After consumption of the starting material (by TLC), the reaction mixture was diluted with water and extracted with EtOAc. Combined organic extracts were dried over anhydrous Na₂SO₄ and concentrated under reduced pressure to obtain crude compound 1 (305 mg, 93%) as yellow solid, which was used for next directly. LC-MS: m/z=579.2 [M+H-Boc]⁺

### (R)-2-amino-N—((S)-3′-(2-((4-fluorobenzyl)((S)-1,1,1-trifluoropropan-2-yl)amino)-2-oxoethyl)-2′,4′-dioxo-2,3-dihydrospiro[indene-1,5′-ozazolidin]-5-yl)-3-methylbutanamide

To a stirring solution of compound 1 (305 mg, 0.45 mmol) in DCM (10 mL) was added TFA (3 mL). The reaction mixture was stirred at room temperature for 1 h. After consumption of the starting material (by TLC), the reaction mixture was concentrated under reduced pressure to obtain crude product, which was purified by silica gel column chromatography eluting with 10% MeOH in DCM to afford product (240 mg, 77%) as a white solid. The product was purified by Chiral HPLC (column: CHIRAL IB; Manufacturer: DAICEL CHIRAL TECHNOLOGIES (CHINA) CO., LTD; Isopropyl alcohol/Hexane (3:7, 0.2% Diethylamine in isopropyl alcohol)) and Prep-HPLC, eluting with a gradient of CH₃CN/H₂O (containing 0.1% TFA) from 10/90 to 70/30 to give the desired product 270-1 (32 mg, 26%) and 270-2 (77 mg, 64%) as TFA salt. LC-MS: m/z=579.2 [M+H]⁺ at RT 15.67 (100% purity). ¹H NMR (400 MHz, CD₃OD) δ 7.79 (s, 1H), 7.55-7.39 (m, 3H), 7.31-7.14 (m, 2H), 7.05 (t, J=8.5 Hz, 1H), 5.51-5.44 (m, 1H), 5.03-4.96 (m, 1H), 4.89-4.76 (m, 1H), 4.65-4.55 (m, 1H), 4.44-4.31 (m 1H), 3.81 (d, J=5.7 Hz, 1H), 3.34-3.24 (m, 2H), 2.85-2.75 (m, 1H), 2.65-2.55 (m, 1H), 2.37-2.26 (m, 1H), 1.43 (dd, J=29.2, 6.9 Hz, 3H), 1.14 (dd, J=12.7, 6.9 Hz, 6H)

### (R)-2-amino-N—((R)-3′-(2-((4-fluorobenzyl)((S)-1,1,1-trifluoropropan-2-yl)amino)-2-oxoethyl)-2′,4′-dioxo-2,3-dihydrospiro[indene-1,5′-ozazolidin]-5-yl)-3-methylbutanamide

LC-MS: m/z=579.2 [M+H]⁺ at RT 17.10 (100% purity). ¹H NMR (400 MHz, CD₃OD) δ 7.79 (s, 1H), 7.55-7.40 (m, 3H), 7.28-7.13 (m, 2H), 7.07-7.01 (m, 1H), 5.58-5.38 (m, 1H), 5.05-4.95 (m, 1H), 4.85-4.75 (m, 1H), 4.63 (d, J=16.9 Hz, 1H), 4.34 (d, J=16.9 Hz, 1H), 3.80 (s, 1H), 3.28-3.13 (m, 2H), 2.86-2.75 (m, 1H), 2.68-2.54 (m, 1H), 2.36-2.25 (m, 1H), 1.43 (dd, J=26.5, 6.9 Hz, 3H), 1.14 (dd, J=12.7, 6.9 Hz, 6H).

### Example 271

### tert-butyl (2R)-1-(3′-(2-(((R)-1-cyclopropyl-2,2,2-trifluoroethyl)(4-fluorobenzyl)amino)-2-oxoethyl)-2′,4′-dioxo-2,3-dihydrospiro[indene-1,5′-ozazolidine]-5-ylamino)-3-methyl-1-oxobutan-2-ylcarbamate (1)

To a stirring solution of intermediate 43 (250 mg, 0.6 mmol) and intermediate 45 (221 mg, 0.6 mmol) in DMF (10 mL) was added K₂CO₃ (124 mg, 0.9 mmol) and stirred at RT for 1 h. After consumption of the starting material (by TLC), the reaction mixture was diluted with water and extracted with EtOAc. Combined organic extracts were dried over anhydrous Na₂SO₄ and concentrated under reduced pressure to obtain crude compound 1 (330 mg, 78%) as a yellow solid, which was used for next directly. LC-MS: m/z=605.2 [M+H-Boc]⁺

### (R)-2-amino-N—((S)-3′-(2-(((R)-1-cyclopropyl-2,2,2-trifluoroethyl)(4-fluorobenzyl)amino)-2-oxoethyl)-2′,4′-dioxo-2,3-dihydrospiro[indene-1,5′-ozazolidine]-5-yl)-3-methylbutanamide

To a stirring solution of compound 1 (330 mg, 0.47 mmol) in DCM (10 mL) was added TFA (3 mL). The reaction mixture was stirred at room temperature for 1 h. After consumption of the starting material (by TLC), the reaction mixture was concentrated under reduced pressure to obtain crude product, which was purified by silica gel column chromatography eluting with 10% MeOH in DCM to afford product (270 mg, 95%) as an off-white solid. The product was purified by SFC (column: CHIRAL CELL OD-H; Manufacturer: DAICEL CHIRAL TECHNOLOGIES (CHINA) CO., LTD; condition: EtOH/CO₂ (3:7, 0.2% FA and 0.2% Diethylamine in EtOH)) and Prep-HPLC, eluting with a gradient of CH₃CN/H₂O (containing 0.1% TFA) from 10/90 to 70/30 to give the desired compound 271-1 (66 mg, 49%) and compound 271-2 (100 mg, 74%) as a TFA salt. LC-MS: m/z=605.2 [M+H]⁺ at RT 3.14 (100% purity). ¹H NMR (400 MHz, CD₃OD) δ 7.80 (s, 1H), 7.54-7.41 (m, 3H), 7.31-7.14 (m, 2H), 7.05-6.98 (m, 1H), 5.11-4.97 (m, 1H), 4.78-4.62 (m, 1H), 4.48-4.37 (m, 2H), 3.94-3.80 (s, 1.5H), 3.30-3.11 (m, 2.5H), 2.87-2.74 (m, 1H), 2.70-2.54 (m, 1H), 2.37-2.25 (m, 1H), 1.36-1.08 (m, 7H), 0.96-0.61 (m, 2H), 0.52-0.14 (m, 2H).

### (R)-2-amino-N—((R)-3′-(2-(((R)-1-cyclopropyl-2,2,2-trifluoroethyl)(4-fluorobenzyl)amino)-2-oxoethyl)-2′,4′-dioxo-2,3-dihydrospiro[indene-,5′-ozazolidine]-5-yl)-3-methylbutanamide

LC-MS: m/z=605.2 [M+H]⁺ at RT 3.82 (100% purity). ¹H NMR (400 MHz, CD₃OD) δ 7.78 (s, 1H), 7.55-7.45 (m, 3H), 7.44-6.91 (m, 3H), 5.12-4.98 (m, 2H), 4.64-4.53 (m, 1H), 4.49-4.39 (m, 1H), 3.92-3.79 (m, 1.5H), 3.32-3.07 (m, 2.5H), 2.87-2.75 (m, 1H), 2.68-2.59 (m 1H), 2.36-2.25 (m, 1H), 1.33-1.05 (m, 7H), 0.87-0.64 (m, 2H), 0.47-0.32 (m, 2H).

### Example 272

### 2-(5-amino-2′,4′-dioxo-2,3-dihydrospiro[indene-1,5′-ozazolidine]-3′-yl)-N—((R)-1-cyclopropyl-2,2,2-trifluoroethyl)-N-(4-fluorobenzyl)acetamide (1)

To a stirring solution of intermediate 45 (552 mg, 1.5 mmol) and intermediate 39 (327 mg, 1.5 mmol) in dry DMF (10 mL) was added K₂CO₃ (621 mg, 4.5 mmol), then stirred at RT for 2 h. Quenched with water, extracted with EtOAc, concentrated under reduced pressure to obtain crude product, which was purified by combiflash (methanol:DCM=1:20) to give compound 1 (0.48 g, 64%). LC-MS: m/z 506.0 [M+H]⁺

### N—((R)-1-cyclopropyl-2,2,2-trifluoroethyl)-N-(4-fluorobenzyl)-2-((S)-5-(3-methylureido)-2′,4′-dioxo-2,3-dihydrospiro[indene-1,5′-ozazolidine]-3′-yl)acetamide: and N—((R)-1-cyclopropyl-2,2,2-trifluoroethyl)-N-(4-fluorobenzyl)-2-((R)-5-(3-methylureido)-2′,4′-dioxo-2,3-dihydrospiro[indene-1,5′-ozazolidine]-3′-yl)acetamide

A solution of compound 1 (487 mg, 0.96 mmol) in THF (20 mL) was added TEA (290 mg, 2.88 mmol) and triphosgen (142 mg, 0.48 mmol) at 0° C. under N₂. The reaction mixture was stirred at room temperature for 0.5 h. Then MeNH₂ (2.4 mL, 4.8 mmol) was added and stirred at RT for 1 h. Quenched with water and extracted with EtOAc, dried and concentrated under reduced pressure to obtain crude product, which was purified by combiflash (methanol:DCM=1:20) to give off-white solid, then purified by SFC (column: CHIRALPAK AS-H; Manufacturer: DAICEL CHIRAL TECHNOLOGIES (CHINA) CO., LTD; condition: EtOH/CO₂ (3:7, 0.2% of FA and 0.2% Diethylamine in EtOH)) to give compound 272-1 (0.13 g, 24%) as a white solid and compound 272-2 (0.14 g, 26%) as a white solid. LC-MS: m/z=563.1 [M+H]⁺ at RT 2.84 (98.85% purity). ¹H NMR (301 MHz, DMSO-d₆) δ 8.71 (s, 1H), 7.64-7.37 (m, 2H), 7.37-7.01 (m, 4H), 6.17-5.99 (m, 1H), 5.12-4.80 (m, 2H), 4.68-4.03 (m, 3H), 3.21-2.92 (m, 2H), 2.80-2.53 (m, 4H), 2.45-2.37 (m, 1H), 1.38-1.15 (m, 1H), 0.87-0.64 (m, 2H), 0.49-0.17 (m, 2H). LC-MS: m/z=563.1 [M+H]⁺ at RT 4.02 (98.78% purity). ¹H NMR (301 MHz, DMSO-d₆) δ 8.71 (s, 1H), 7.64-7.39 (m, 2H), 7.35-7.05 (m, 4H), 6.16-6.03 (m, 1H), 5.11-4.77 (m, 2H), 4.65-4.03 (m, 3H), 3.20-2.89 (m, 2H), 2.79-2.54 (m, 4H), 2.46-2.34 (m, 1H), 1.40-1.14 (m, 1H), 0.86-0.63 (m, 2H), 0.48-0.14 (m, 2H).

### Example 273

### tert-butyl 3-(3′-(2-(((R)-1-cyclopropyl-2,2,2-trifluoroethyl)(4-fluorobenzyl)amino)-2-oxoethyl)-2′,4′-dioxo-2,3-dihydrospiro[indene-1,5′-ozazolidine]-5-ylcarbamoyl)azetidine-1-carboxylate (1)

To a stirring solution of intermediate 45 (229 mg, 0.62 mmol) and intermediate 48 (250 mg, 0.62 mmol) in dry DMF (10 mL) was added K₂CO₃ (256 mg, 1.68 mmol), then stirred at RT for 2 h. Quenched with water, extracted with EtOAc, concentrated under reduced pressure to obtain crude product, which was purified by combiflash (methanol:DCM=1:20) to give compound 1 (0.4 g, 95%). LC-MS: m/z 589.0 [M+H-Boc]⁺

### N—((S)-3′-(2-(((R)-1-cyclopropyl-2,2,2-trifluoroethyl)(4-fluorobenzyl)amino)-2-oxoethyl)-2′,4′-dioxo-2,3-dihydrospiro[indene-1,5′-ozazolidine]-5-yl)azetidine-3-carboxamide; and N—((R)-3′-(2-(((R)-1-cyclopropyl-2,2,2-trifluoroethyl)(4-fluorobenzyl)amino)-2-oxoethyl)-2′,4′-dioxo-2,3-dihydrospiro[indene-1,5′-ozazolidine]-5-yl)azetidine-3-carboxamide

A solution of compound 1 (400 mg, 0.58 mmol) in DCM (20 mL) was added TFA (1 mL). The reaction mixture was stirred at room temperature for 1 h. Concentrated under reduced pressure to obtain crude product, which was purified by combiflash (methanol:DCM=1:20) to give off-white solid, then purified by SFC (column: CHIRALPAK OD-H; Manufacturer: DAICEL CHIRAL TECHNOLOGIES (CHINA) CO., LTD; condition: EtOH/CO₂ (3:7, 0.2% of FA and 0.2% Diethylamine in EtOH)) to give compound 273-1 (50 mg, 15%) as a white solid and compound 273-2 (60 mg, 18%) as a white solid. LC-MS: m/z=589.1 [M+H]⁺ at RT 3.69 (98.0% purity). ¹H NMR (400 MHz, CD₃OD)₆ 7.80 (s, 1H), 7.58-7.37 (m, 3H), 7.37-6.91 (m, 3H), 5.47-4.93 (m, 2H), 4.78-4.16 (m, 6H), 4.03-3.70 (m, 1H), 3.29-3.03 (m, 2H), 2.85-2.69 (m, 1H), 2.65-2.50 (m, 1H), 1.25-1.10 (m, 1H), 0.93-0.59 (m, 2H), 0.47-0.12 (m, 2H). LC-MS: m/z=589.1 [M+H]⁺ at RT 4.69 (95.4% purity). ¹H NMR (400 MHz, CD₃OD) δ 7.79 (s, 1H), 7.55-7.24 (m, 4H), 7.22-6.99 (m, 2H), 5.21-4.90 (m, 2H), 4.72-4.20 (m, 6H), 3.95-3.75 (m, 1H), 3.30-3.04 (m, 2H), 2.90-2.70 (m, 1H), 2.68-2.52 (m, 1H), 1.25-1.12 (m, 1H), 0.92-0.62 (m, 2H), 0.52-0.21 (m, 2H).

### Example 274

### tert-butyl (2R)-1-(3′-(2-(((R)-1-cyclopropyl-2,2,2-trifluoroethyl)(4-fluorobenzyl)amino)-2-oxoethyl)-2′,4′-dioxo-2,3-dihydrospiro[indene-1,5′-ozazolidine]-5-ylamino)-3-methyl-1-oxobutan-2-ylcarbamate (1)

To a stirring solution of Intermediate 43 (200 mg, 0.48 mmol) and Intermediate 45 (176 mg, 0.48 mmol) in DMF (3 mL) was added K₂CO₃ (99 mg, 0.72 mmol) and stirred at room temperature for 1 h. After consumption of the starting material (by TLC), the reaction mixture was diluted with water and extracted with EtOAc. Combined organic extracts were dried over anhydrous Na₂SO₄ and concentrated under reduced pressure to obtain crude compound 1 (314 mg, 93%) as a yellow solid, which was used for next step directly. LC-MS: m/z=605.2 [M+H-Boc]⁺

### (2R)-2-amino-N-(3′-(2-(((R)-1-cyclopropyl-2,2,2-trifluoroethyl)(4-fluorobenzyl)amino)-2-oxoethyl)-2′,4′-dioxo-2,3-dihydrospiro[indene-1,5′-ozazolidine]-5-yl)-3-methylbutanamide

To a stirring solution of compound 1 (314 mg, 0.44 mmol) in DCM (15 mL) was added TFA (3 mL). The reaction mixture was stirred at room temperature for 1 h. After consumption of the starting material (by TLC), the reaction mixture was concentrated under reduced pressure to obtain crude product, which was purified by silica gel column chromatography eluting with 10% CH₃OH in DCM to afford compound 274-1 (211 mg, 66%) as a white solid. LC-MS: m/z=605.2 [M+H]⁺ at RT 4.57 (97.3% purity). ¹H NMR (301 MHz, DMSO-d₆) δ 10.64 (s, 1H), 8.24 (s, 2H), 7.73 (s, 1H), 7.56-7.36 (m, 3H), 7.27 (t, J=8.7 Hz, 2H), 7.19-7.04 (m, 1H), 5.09-4.81 (m, 2H), 4.63-4.52 (m, 1H), 4.38-4.22 (m, 1H), 3.77 (s, 0.5H), 3.27-2.97 (m, 2.5H), 2.72-2.61 (m, 2H), 2.24-2.15 (m, 2H), 1.29-1.21 (m, 2H), 1.11-0.93 (m, 5H), 0.85-0.72 (m, 2H), 0.44-0.18 (m, 2H).

### Example 275

### tert-butyl 3-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-ylcarbamoyl)azetidine-1-carboxylate (1)

To a stirring solution of intermediate 48 (280 mg, 0.7 mmol) and intermediate 44 (240 mg, 0.7 mmol) in DMF (3 mL) was added K₂CO₃ (145 mg, 1.05 mmol) and stirred at room temperature for 1 h. After consumption of the starting material (by TLC), the reaction mixture was diluted with water and extracted with EtOAc. Combined organic extracts were dried over anhydrous Na₂SO₄ and concentrated under reduced pressure to obtain crude compound 1 (380 mg, 85%) as a yellow solid, which was used for next step directly. LC-MS: m/z=535.2 [M+H-Boc]⁺

### N—((S)-3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)azetidine-3-carboxamide

To a stirring solution of compound 1 (380 mg, 0.6 mmol) in DCM (10 mL) was added TFA (2 mL). The reaction mixture was stirred at room temperature for 1 h. After consumption of the starting material (by TLC), the reaction mixture was concentrated under reduced pressure to obtain crude product, which was purified by silica gel column chromatography eluting with 10% MeOH in DCM to afford product (270 mg, 84%) as an off-white solid. The product was purified by SFC (column: CHIRAL CELL OD-H; Manufacturer: DAICEL CHIRAL TECHNOLOGIES (CHINA) CO., LTD; EtOH/CO₂ (3:7, 0.2% FA and 0.2% Diethylamine in EtOH)) and Prep-HPLC, eluting with a gradient of CH₃CN/H₂O (containing 0.1% TFA) from 10/90 to 70/30 to give the desired product compound 275-1 (111 mg, 57%) compound 275-2 (112 mg, 57%) as TFA salt. LC-MS: m/z=535.2 [M+H]⁺ at RT 6.52 (100% purity). ¹H NMR (400 MHz, CD₃OD) δ 7.80 (s, 1H), 7.56-7.42 (m, 3H), 7.32 (dd, J=8.4, 5.4 Hz, 1H), 7.15 (t, J=8.7 Hz, 1H), 7.02 (t, J=8.8 Hz, 1H), 4.83-4.70 (m, 2H), 4.69-4.41 (m, 2H), 4.41-4.23 (m, 4H), 3.91-3.77 (m, 1.5H), 3.38-3.34 (m, 0.5H), 3.29-3.22 (m, 1H), 3.19-3.11 (m, 1H), 2.87-2.75 (m, 1H), 2.65-2.53 (m, 1H), 1.29 (dd, J=35.1, 6.7 Hz, 3H), 1.06-0.89 (m, 1H), 0.69-0.53 (m, 1H), 0.48-0.19 (m, 3H).

### N—((R)-3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)azetidine-3-carboxamide

LC-MS: m/z=535.2 [M+H]⁺ at RT 8.45 (96.9% purity). ¹H NMR (400 MHz, CD₃OD) δ 7.80 (s, 1H), 7.57-7.42 (m, 3H), 7.32 (dd, J=8.1, 5.5 Hz, 1H), 7.15 (t, J=8.7 Hz, 1H), 7.03 (t, J=8.7 Hz, 1H), 4.85-4.74 (m, 2H), 4.65-4.43 (m, 2H), 4.39-4.25 (m, 4H), 3.90-3.77 (m, 1.5H), 3.39-3.34 (m, 0.5H), 3.28-3.21 (m, 1H), 3.19-3.06 (m, 1H), 2.87-2.79 (m, 1H), 2.66-2.53 (m, 1H), 1.29 (dd, J=34.3, 6.7 Hz, 3H), 1.02-0.94 (dd, J=8.5, 4.7 Hz, 1H), 0.70-0.55 (m, 1H), 0.46-0.21 (m, 3H).

### Example 276

### tert-butyl (2S)-1-(3′-(2-(((R)-1-cyclopropyl-2,2,2-trifluoroethyl)(4-fluorobenzyl)amino)-2-oxoethyl)-2′,4′-dioxo-2,3-dihydrospiro[indene-1,5′-ozazolidine]-5-ylamino)-3-methyl-1-oxobutan-2-ylcarbamate (1)

To a stirring solution of intermediate 52 (270 mg, 0.65 mmol) and intermediate 45 (238 mg, 0.65 mmol) in dry DMF (10 mL) was added K₂CO₃ (269 mg, 1.95 mmol), then stirred at RT for 2 h. Quenched with water, extracted with EtOAc, concentrated under reduced pressure to obtain crude product, which was purified by combiflash (methanol:DCM=1:20) to give compound 1 (0.46 g, 100%). LC-MS: m/z 605.1 [M+H-Boc]⁺

### (2S)-2-amino-N-(3′-(2-(((R)-1-cyclopropyl-2,2,2-trifluoroethyl)(4-fluorobenzyl)amino)-2-oxoethyl)-2′,4′-dioxo-2,3-dihydrospiro[indene-1,5′-ozazolidine]-5-yl)-3-methylbutanamide

A solution of compound 1 (460 mg, 0.65 mmol) in DCM (20 mL) was added TFA (1 mL). The reaction mixture was stirred at room temperature for 1 h. Concentrated under reduced pressure to obtain crude product, which was purified by Prep-HPLC, eluting with a gradient of CH₃CN/H₂O (containing 0.1% TFA) from 10/90 to 70/30 to give the desired product compound 276-1 (220 mg, 55%) as a TFA salt. LC-MS: m/z=605.2 [M+H]⁺ at RT 4.53 (98.05% purity). ¹H NMR (400 MHz, DMSO-d₆) δ 10.71 (s, 1H), 8.30 (s, 3H), 7.82-7.68 (m, 1H), 7.63-7.04 (m, 6H), 5.12-4.82 (m, 2H), 4.69-4.04 (m, 3H), 3.86-3.73 (m, 1H), 3.29-2.98 (m, 2H), 2.76-2.53 (m, 2H), 2.23-2.11 (m, 1H), 1.37-1.13 (m, 1H), 1.07-0.87 (m, 6H), 0.83-0.59 (m, 2H), 0.49-0.11 (m, 2H).

### Example 277

### tert-butyl (2S)-1-(3′-(2-((4-fluorobenzyl)((S)-1,1,1-trifluoropropan-2-yl)amino)-2-oxoethyl)-2′,4′-dioxo-2,3-dihydrospiro[indene-1,5′-ozazolidine]-5-ylamino)-3-methyl-1-oxobutan-2-ylcarbamate (1)

To a stirring solution of intermediate 52 (270 mg, 0.65 mmol) and intermediate 45 (222 mg, 0.65 mmol) in dry DMF (10 mL) was added K₂CO₃ (269 mg, 1.95 mmol), then stirred at RT for 2 h. Quenched with water, extracted with EtOAc, concentrated under reduced pressure to obtain crude product, which was purified by combiflash (methanol:DCM=1:20) to give compound 1 (0.39 g, 88%). LC-MS: m/z 579.1 [M+H-Boc]⁺

### (2S)-2-amino-N-(3′-(2-((4-fluorobenzyl)((S)-1,1,1-trifluoropropan-2-yl)amino)-2-oxoethyl)-2′,4′-dioxo-2,3-dihydrospiro[indene-1,5′-ozazolidine]-5-yl)-3-methylbutanamide

A solution of compound 1 (390 mg, 0.57 mmol) in DCM (20 mL) was added TFA (1 mL). The reaction mixture was stirred at room temperature for 1 h. Concentrated under reduced pressure to obtain crude product, which was purified by Prep-HPLC, eluting with a gradient of CH₃CN/H₂O (containing 0.1% TFA) from 10/90 to 70/30 to give the desired product compound 277-1 (210 mg, 63%) as a TFA salt. LC-MS: m/z=579.2 [M+H]⁺ at RT 4.34 (96.7% purity). ¹H NMR (400 MHz, DMSO-d₆) δ 10.68 (s, 1H), 8.28 (s, 3H), 7.84-7.71 (m, 1H), 7.65-7.04 (m, 6H), 5.53-4.39 (m, 4H), 4.37-4.13 (m, 1H), 3.87-3.72 (m, 1H), 3.33-3.00 (m, 2H), 2.87-2.58 (m, 2H), 2.32-2.10 (m, 1H), 1.47-1.30 (m, 3H), 1.14-0.88 (m, 6H).

### Example 278

### tert-butyl (2R)-1-(3′-(2-((4-fluorobenzyl)((S)-1,1,1-trifluoropropan-2-yl)amino)-2-oxoethyl)-2′,4′-dioxo-2,3-dihydrospiro[indene-1,5′-ozazolidine]-5-ylamino)-3-methyl-1-oxobutan-2-ylcarbamate (1)

To a stirring solution of Intermediate 43 (200 mg, 0.48 mmol) and Intermediate 46 (164 mg, 0.48 mmol) in DMF (3 mL) was added K₂CO₃ (99 mg, 0.72 mmol) and stirred at room temperature for 1 h. After consumption of the starting material (by TLC), the reaction mixture was diluted with water and extracted with EtOAc. Combined organic extracts were dried over anhydrous Na₂SO₄ and concentrated under reduced pressure to obtain crude compound 1 (296 mg, 91%) as a yellow solid, which was used for next step directly. LC-MS: m/z=579.2 [M+H-Boc]⁺

### (2R)-2-amino-N-(3′-(2-((4-fluorobenzyl)((S)-1,1,1-trifluoropropan-2-yl)amino)-2-oxoethyl)-2′,4′-dioxo-2,3-dihydrospiro[indene-1,5′-ozazolidine]-5-yl)-3-methylbutanamide

To a stirring solution of compound 1 (296 mg, 0.44 mmol) in DCM (15 mL) was added TFA (3 mL). The reaction mixture was stirred at room temperature for 1 h. After consumption of the starting material (by TLC), the reaction mixture was concentrated under reduced pressure to obtain crude product, which was purified by Prep-HPLC, eluting with a gradient of CH₃CN/H₂O (containing 0.1% TFA) from 10/90 to 70/30 to give the desired product compound 278-1 (211 mg, 69%) as TFA salt. LC-MS: m/z=579.2 [M+H]⁺ at RT 4.36 (99.6% purity). ¹H NMR (400 MHz, DMSO-d₆) δ 10.65 (s, 1H), 8.26 (s, 2H), 7.74 (s, 1H), 7.53-7.38 (m, 3H), 7.35-7.10 (m, 3H), 5.42 (s, 1H), 5.07-4.96 (m, 1H), 4.85-4.72 (m, 1H), 4.68-4.57 (m, 2H), 4.23 (dd, J=17.0, 5.8 Hz, 1H), 3.78 (s, 1H), 3.24-3.08 (m, 2H), 2.67 (dd, J=15.4, 7.7 Hz, 1H), 2.20 (s, 1H), 1.45-1.33 m, 3H), 1.01 (t, J=6.3 Hz, 6H).

### Example 279

### tert-butyl 3-(3′-(2-((4-fluorobenzyl)((S)-1,1,1-trifluoropropan-2-yl)amino)-2-oxoethyl)-2′,4′-dioxo-2,3-dihydrospiro[indene-1,5′-ozazolidine]-5-ylcarbamoyl)azetidine-1-carboxylate (1)

To a stirring solution of Intermediate 48 (250 mg, 0.62 mmol) and Intermediate 46 (212 mg, 0.62 mmol) in DMF (3 mL) was added K₂CO₃ (129 mg, 0.93 mmol) and stirred at room temperature for 1 h. After consumption of the starting material (by TLC), the reaction mixture was diluted with water and extracted with EtOAc. Combined organic extracts were dried over anhydrous Na₂SO₄ and concentrated under reduced pressure to obtain crude compound 1 (374 mg, 91%) which was used for next step directly. LC-MS: m/z=563.2 [M+H-Boc]⁺

### N—((S)-3′-(2-((4-fluorobenzyl)((S)-1,1,1-trifluoropropan-2-yl)amino)-2-oxoethyl)-2′,4′-dioxo-2,3-dihydrospiro[indene-1,5′-ozazolidine]-5-yl)azetidine-3-carboxamide

To a stirring solution of compound 1 (374 mg, 0.56 mmol) in DCM (10 mL) was added TFA (3 mL). The reaction mixture was stirred at room temperature for 1 h. After consumption of the starting material (by TLC), the reaction mixture was concentrated under reduced pressure to obtain crude product, which was purified by Prep-HPLC, eluting with a gradient of CH₃CN/H₂O (containing 0.1% TFA) from 10/90 to 70/30 to give the desired product compound 279-1 (260 mg, 68%) as a TFA salt. LC-MS: m/z=563.2 [M+H]⁺ at RT 4.15 (97.8% purity). ¹H NMR (301 MHz, CD₃OD) δ 7.77 (s, 1H), 7.50-7.34 (m, 3H), 7.25-7.15 (m, 2H), 7.05-6.93 (d, J=6.0 Hz, 1H), 5.50-5.39 (m, 1H), 4.99-4.93 (m, 1H), 4.83-4.76 (m, 1H), 4.72-4.65 (m, 1H), 4.59-4.52 (m, 1H), 4.40-4.19 (m, 4H), 3.88-3.75 (m, 1H), 3.25-3.03 (m, 2H), 2.84-2.69 (m, 1H), 2.62-2.51 (m, 1H), 1.45-1.27 (m, 3H).

### Example 281

### 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

### Example 281A

### (S)-1,1,1-trifluoro-N-(4-fluorobenzyl)propan-2-amine

A 500 ml flask was charged with (S)-1,1,1-trifluoropropan-2-amine hydrochloride (23 g, 154 mmol) and N,N-dimethylformamide (150 ml). The mixture was stirred until the entire white solid dissolved. To the resulting solution was added potassium carbonate (85 g, 615 mmol), while maintaining the temperature, and the mixture was stirred for 10 minutes. To the mixture at 22.4° C. was added 1-(bromomethyl)-4-fluorobenzene (22.04 ml, 177 mmol) rapidly dropwise over 9 minutes with a temperature rise to 24.8° C. The white suspension was stirred overnight under nitrogen at ambient temperature. The reaction mixture was placed in a cool water bath and was quenched at 15° C. with brine. The quench was exothermic and the temperature was kept below 25° C. The white suspension was partitioned between water and ethyl acetate. The layers were separated and the aqueous layer was extracted with ethyl acetate. The organic layer was dried over sodium sulfate, filtered and concentrated. Silica gel column chromatography provided the title compound.

### Example 281B

### (S)-2-bromo-N-(4-fluorobenzyl)-N-(1,1,1-trifluoropropan-2-yl)acetamide

A 1000 ml flask was charged with (S)-1,1,1-trifluoro-N-(4-fluorobenzyl)propan-2-amine (17.53 g, 79 mmol) and dichloromethane (300 ml). The resulting solution was cooled in an ice bath to 1.0° C. and 2-bromoacetyl bromide (7.94 ml, 91 mmol) was added dropwise over 6 minutes. Triethylamine (14.36 ml, 103 mmol) was added dropwise over 10 minutes while the temperature was below 5° C. by adjusting the rate of addition. The slightly brown reaction mixture was stirred overnight under nitrogen while warming to ambient temperature. Additional 2-bromoacetyl bromide (1.4 ml, 15.8 mmol, 0.2 equiv) was added dropwise followed by the addition of triethylamine (5.5 ml, 39.5 mmol, 0.5 equiv). The reaction mixture was stirred an additional 4 hours, quenched with saturated sodium bicarbonate (100 ml) and the layers were partitioned between ethyl acetate and brine. The separated aqueous layer was extracted with ethyl acetate and the combined organic layers were dried over sodium sulfate, filtered and concentrated. Silica gel column chromatography provided the title compound.

### Example 281C

### rac-5-bromo-1-((trimethylsilyl)oxy)-2,3-dihydro-1H-indene-1-carbonitrile

5-Bromo-2,3-dihydro-1H-inden-1-one (50 g, 237 mmol) was taken up in methylene chloride (237 ml) and N-methylmorpholine N-oxide (8.33 g, 71.1 mmol) was added followed by trimethyl cyanide (38.1 ml, 284 mmol). The resulting black solution was stirred for 5 days and treated with saturated sodium bicarbonate; the aqueous layer was extracted with dichloromethane and the combined organic layers were dried (MgSO₄), filtered and concentrated. The residue was purified by silica gel column chromatography to give the title compound.

### Example 281D

### rac-ethyl 5-bromo-1-hydroxy-2,3-dihydro-1H-indene-1-carbimidate

A three neck 3 L flask was fitted with a hastelloy thermocouple, HCl gas in (diffussor) and HCl out, and vented out to the back of the hood. 5-Bromo-1-((trimethylsilyl)oxy)-2,3-dihydro-1H-indene-1-carbonitrile (52 g, 168 mmol) was taken up in ethanol (1117 ml) and cooled to 0° C. (internal temp). HCl (g) (227 g, 6226 mmol) was gassed in carefully (backflow preventer). The lecture bottle was emptied over 2 hours while the internal temperature was kept below 25° C. with an ice bath. The mixture was stirred for an additional hour and then concentrated. The solids were washed with diethyl ether three times and dried with MgSO₄ to afford the title compound.

### Example 281E

### (R)-5-bromo-2,3-dihydrospiro[indene-1,5′-oxazolidine]-2′,4′-dione and (S)-5-bromo-2,3-dihydrospiro[indene-1,5′-oxazolidine]-2′,4′-dione

rac-Ethyl 5-bromo-1-hydroxy-2,3-dihydro-1H-indene-1-carbimidate hydrochloride (49.5 g, 154 mmol) was suspended in tetrahydrofuran (772 ml) and triethylamine (64.6 ml, 463 mmol) was added and the solution was cooled to 0° C. with an ice bath. Triphosgene (45.8 g, 154 mmol) (CAUTION, was weighed out in hood) was added, keeping the internal temperature below 25° C. (CAUTION, sharp exotherm was observed), and the mixture was warmed to room temperature and stirred for about 1 hour. The reaction mixture was cooled to 6-8° C., carefully diluted with aqueous 1N HCl (exotherm), stirred for 60 minutes, and then extracted with ethyl acetate. The aqueous layer was extracted with ethyl acetate and the combined organics were dried (MgSO₄), filtered, treated with charcoal, filtered through celite and concentrated. The solids were triturated with dichloromethane and the racemic product was purified by chiral SFC column to provide both (R)-5-bromo-2,3-dihydrospiro[indene-1,5′-oxazolidine]-2′,4′-dione (eluted first) and (S)-5-bromo-2,3-dihydrospiro[indene-1,5′-oxazolidine]-2′,4′-dione (eluted second). Single crystal x-ray diffraction determined the absolute configuration of each.

### Example 281F

### 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

(R)-5-bromo-2,3-dihydrospiro[indene-1,5′-oxazolidine]-2′,4′-dione (1.389 g, 4.92 mmol) and (S)-2-bromo-N-(4-fluorobenzyl)-N-(1,1,1-trifluoropropan-2-yl)acetamide (1.853 g, 5.42 mmol) were combined in a 100 ml round-bottom flask and N,N-dimethylformamide (16.41 ml) was added followed by potassium carbonate (1.361 g, 9.85 mmol). The flask was flushed with nitrogen and stirred overnight. The reaction mixture was diluted with water and ethyl acetate. The layers were separated and the aqueous layer was extracted with ethyl acetate. The combined organic layers were dried (MgSO₄), filtered and concentrated. The residue was purified by silica gel column chromatography to give the title compound. ¹H NMR (400 MHz, DMSO-d₆, Temp=90° C.) δ ppm 7.62 (d, J=1.7 Hz, 1H), 7.51 (dd, J=8.3, 1.8 Hz, 1H), 7.42-7.24 (m, 3H), 7.15 (t, J=8.6 Hz, 2H), 5.22 (s, 1H), 4.85 (d, J=17.5 Hz, 1H), 4.63 (s, 2H), 4.37 (s, 1H), 3.26-3.03 (m, 2H), 2.69 (ddd, J=14.9, 8.5, 6.4 Hz, 1H), 2.63-2.50 (m, 1H), 1.36 (d, J=6.9 Hz, 3H); MS (ESI(+)) m/e 565 (M+Na)⁺.

### Example 282

### N-[(1R)-3′-(2-{[(1R)-1-cyclopropylethyl](4-fluorobenzyl)amino}-2-oxoethyl)-2′,4′-dioxo-2,3-dihydrospiro[indene-1,5′-[1,3]oxazolidin]-5-yl]-3,3-difluoroazetidine-1-carboxamide

2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide (30 mg, 0.066 mmol) was taken up in dichloromethane (664 μl). Triphosgene (9.86 mg, 0.033 mmol) was added followed by triethylamine (13.89 μl, 0.100 mmol). The reaction was stirred for 2.5 hours and 3,3-difluoroazetidine hydrochloride (86 mg, 0.664 mmol) was added followed by triethylamine (93 μl, 0.664 mmol). The mixture was stirred for 2.5 days and silica gel column chromatography (no workup) provided the title compound. ¹H NMR (400 MHz, DMSO-d₆) δ ppm 8.73 (s, 1H), 7.63-7.01 (m, 7H), 4.68 (s, 2H), 3.21-3.01 (m, 3H), 2.75-2.60 (m, 1H), 1.26 (s, 4H), 1.22-1.12 (m, 4H), 1.03-0.76 (m, 4H), 0.50 (t, J=8.2 Hz, 1H), 0.26 (tt, J=9.2, 4.9 Hz, 3H); MS (ESI(+)) m/e 593 (M+Na)⁺.

### Example 283

### N-benzyl-2-[(1R)-5-bromo-2′,4′-dioxo-2,3-dihydro-3′H-spiro[indene-1,5′-[1,3]oxazolidin]-3′-yl]-N-[(1S)-1-cyclopropylethyl]acetamide

### Example 283A

### (S)—N-benzyl-1-cyclopropylethanamine

(S)-1-cyclopropylethanamine (6.26 ml, 58.7 mmol) was dissolved in methanol (147 ml) and benzaldehyde (11.94 ml, 117 mmol) was added. The solution was stirred for 10 minutes at room temperature, cooled to 0° C., and treated with sodium triacetoxyborohydride (24.89 g, 117 mmol) in a single portion. The mixture was allowed to warm to room temperature and stirred overnight after which it was treated with 25 ml aqueous 1N NaOH. Concentration followed by dilution with aqueous 1N NaOH and ethyl acetate gave a bilayer mixture and the separated aqueous layer was extracted three times with ethyl acetate. The combined organic layers were dried (MgSO₄), filtered and concentrated. The residue was purified by silica gel column chromatography to afford the title compound.

### Example 283B

### (S)—N-benzyl-2-bromo-N-(1-cyclopropylethyl)acetamide

The title compound was prepared as described in EXAMPLE 281B, substituting (S)—N-benzyl-1-cyclopropylethanamine for (S)-1,1,1-trifluoro-N-(4-fluorobenzyl)propan-2-amine.

### Example 283C

### N-benzyl-2-[(1R)-5-bromo-2′,4′-dioxo-2,3-dihydro-3′H-spiro[indene-1,5′-[1,3]oxazolidin]-3′-yl]-N-[(1S)-1-cyclopropylethyl]acetamide

The title compound was prepared as described in EXAMPLE 281F, substituting (S)—N-benzyl-2-bromo-N-(1-cyclopropylethyl)acetamide for (S)-2-bromo-N-(4-fluorobenzyl)-N-(1,1,1-trifluoropropan-2-yl)acetamide. ¹H NMR (400 MHz, DMSO-d₆) δ ppm 7.50 (t, J=1.8 Hz, 1H), 7.39 (ddd, J=10.6, 8.2, 1.8 Hz, 1H), 7.34-7.17 (m, 3H), 7.17-6.94 (m, 3H), 4.62 (s, 1H), 4.58-4.40 (m, 1H), 4.37-4.11 (m, 2H), 3.62 (dd, J=9.6, 6.8 Hz, 1H), 3.11-2.83 (m, 2H), 2.50 (ddd, J=14.9, 8.5, 6.6 Hz, 1H), 2.44-2.34 (m, 1H), 1.08-0.91 (m, 3H), 0.80 (dtd, J=14.0, 7.0, 5.8, 3.2 Hz, 1H), 0.42-−0.02 (m, 4H); MS (DCI(+)) m/e 514 (M+NH₄)⁺.

### Example 284

### 2-[(1S)-5-amino-2′,4′-dioxo-2,3-dihydro-3′H-spiro[indene-1,5′-[1,3]oxazolidin]-3′-yl]-N-[(1S)-1-cyclopropylethyl]-N-(4-fluorobenzyl)acetamide

### Example 284A

### (S)-1-cyclopropyl-N-(4-fluorobenzyl)ethanamine

The title compound was prepared as described in EXAMPLE 283A, substituting (S)-1-cyclopropylethanamine for (S)-1-cyclopropylethanamine and 4-fluorobenzaldehyde for benzaldehyde.

### Example 284B

### (S)-2-bromo-N-(1-cyclopropylethyl)-N-(4-fluorobenzyl)acetamide

The title compound was prepared as described in EXAMPLE 281B, substituting (S)-1-cyclopropyl-N-(4-fluorobenzyl)ethanamine for (S)-1,1,1-trifluoro-N-(4-fluorobenzyl)propan-2-amine.

### Example 284C

### 2-((S)-5-bromo-2′,4′-dioxo-2,3-dihydrospiro[indene-1,5′-oxazolidin]-3′-yl)-N—((S)-1-cyclopropylethyl)-N-(4-fluorobenzyl)acetamide

The title compound was prepared as described in EXAMPLE 281F, substituting (S)-5-bromo-2,3-dihydrospiro[indene-1,5′-oxazolidine]-2′,4′-dione for (R)-5-bromo-2,3-dihydrospiro[indene-1,5′-oxazolidine]-2′,4′-dione and (S)-2-bromo-N-(1-cyclopropylethyl)-N-(4-fluorobenzyl)acetamide for (S)-2-bromo-N-(4-fluorobenzyl)-N-(1,1,1-trifluoropropan-2-yl)acetamide.

### Example 284D

### N—((S)-1-cyclopropylethyl)-2-((S)-5-((diphenylmethylene)amino)-2′,4′-dioxo-2,3-dihydrospiro[indene-1,5′-oxazolidin]-3′-yl)-N-(4-fluorobenzyl)acetamide

Palladium(II) acetate (2.76 mg, 0.012 mmol) and (2,2′-bis(diphenylphosphino)-1,1′-binaphthyl) (17.24 mg, 0.028 mmol) were placed in a reaction vial and the vial was flushed with nitrogen. N-Benzyl-2-((S)-5-bromo-2′,4′-dioxo-2,3-dihydrospiro[indene-1,5′-oxazolidin]-3′-yl)-N—((S)-1-cyclopropylethyl)acetamide (153 mg, 0.308 mmol), diphenylmethanimine (78 mg, 0.431 mmol), cesium carbonate (140 mg, 0.431 mmol) and toluene (2051 μl) were added and the reaction was stirred and heated at 100° C. for 3.5 hours. The mixture was cooled and silica gel column chromatography (no work up) gave the title compound.

### Example 284E

### 2-[(1S)-5-amino-2′,4′-dioxo-2,3-dihydro-3′H-spiro[indene-1,5′-[1,3]oxazolidin]-3′-yl]-N-[(1S)-1-cyclopropylethyl]-N-(4-fluorobenzyl)acetamide

N—((S)-1-Cyclopropylethyl)-2-((S)-5-((diphenylmethylene)amino)-2′,4′-dioxo-2,3-dihydrospiro[indene-1,5′-oxazolidin]-3′-yl)-N-(4-fluorobenzyl)acetamide (273 mg, 0.443 mmol) was placed in a vial in tetrahydrofuran (4 ml). Hydrogen chloride (1.552 ml, 3.10 mmol) was added and the reaction was stirred for 2 hours. Concentration and silica gel column chromatography afforded the title compound. ¹H NMR (400 MHz, DMSO-d₆, Temp=90° C.) δ ppm 7.36 (s, 2H), 7.11 (s, 2H), 7.05 (d, J=8.3 Hz, 1H), 6.61-6.41 (m, 2H), 5.13 (s, 2H), 4.67 (s, 2H), 4.37 (s, 2H), 3.09-2.99 (m, 2H), 2.88 (ddd, J=16.2, 8.7, 3.6 Hz, 1H), 2.60 (ddd, J=15.3, 8.7, 7.0 Hz, 1H), 2.40 (ddd, J=14.4, 8.1, 3.7 Hz, 1H), 1.22-1.10 (m, 3H), 0.96 (dd, J=8.4, 4.5 Hz, 1H), 0.85 (d, J=6.9 Hz, 1H), 0.49 (d, J=5.7 Hz, 1H), 0.26 (ddd, J=15.3, 9.9, 5.8 Hz, 3H); MS (ESI(+)) m/e 474 (M+Na)⁺.

### Example 285

### 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide

### Example 285A

### (R)-1-cyclopropyl-N-(4-fluorobenzyl)ethanamine

The title compound was prepared as described in EXAMPLE 283A, substituting (R)-1-cyclopropylethanamine for (S)-1-cyclopropylethanamine and 4-fluorobenzaldehyde for benzaldehyde.

### Example 285B

### (R)-2-bromo-N-(1-cyclopropylethyl)-N-(4-fluorobenzyl)acetamide

The title compound was prepared as described in EXAMPLE 281B, substituting (R)-1-cyclopropyl-N-(4-fluorobenzyl)ethanamine for (S)-1,1,1-trifluoro-N-(4-fluorobenzyl)propan-2-amine.

### Example 285C

### 2-((R)-5-bromo-2′,4′-dioxo-2,3-dihydrospiro[indene-1,5′-oxazolidin]-3′-yl)-N—((R)-1-cyclopropylethyl)-N-(4-fluorobenzyl)acetamide

The title compound was prepared as described in EXAMPLE 281F, substituting (R)-5-bromo-2,3-dihydrospiro[indene-1,5′-oxazolidine]-2′,4′-dione for (R)-5-bromo-2,3-dihydrospiro[indene-1,5′-oxazolidine]-2′,4′-dione and (R)-2-bromo-N-(1-cyclopropylethyl)-N-(4-fluorobenzyl)acetamide for (S)-2-bromo-N-(4-fluorobenzyl)-N-(1,1,1-trifluoropropan-2-yl)acetamide.

### Example 285D

### 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide

The title compound was prepared as described in EXAMPLE 284D-E, substituting 2-((R)-5-bromo-2′,4′-dioxo-2,3-dihydrospiro[indene-1,5′-oxazolidin]-3′-yl)-N—((R)-1-cyclopropylethyl)-N-(4-fluorobenzyl)acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide in EXAMPLE 284D. ¹H NMR (400 MHz, DMSO-d₆, Temp=90° C.) δ ppm 7.36 (s, 2H), 7.11 (s, 2H), 7.05 (d, J=8.2 Hz, 1H), 6.57-6.42 (m, 2H), 5.13 (s, 2H), 4.67 (s, 2H), 4.37 (s, 2H), 3.03 (d, J=8.1 Hz, 3H), 2.97-2.80 (m, 1H), 2.60 (ddd, J=15.2, 8.7, 6.9 Hz, 1H), 2.41 (ddd, J=14.5, 8.2, 3.7 Hz, 1H), 1.18 (d, J=6.6 Hz, 3H), 0.96 (dq, J=8.3, 4.1, 3.1 Hz, 1H), 0.86 (t, J=6.5 Hz, 1H), 0.50 (d, J=5.1 Hz, 1H), 0.35-0.12 (m, 3H); MS (ESI(+)) m/e 474 (M+Na)⁺.

### Example 286

### 2-[(1R)-5-amino-2′,4′-dioxo-2,3-dihydro-3′H-spiro[indene-1,5′-[1,3]oxazolidin]-3′-yl]-N-benzyl-N-[(1S)-1-cyclopropylethyl]acetamide

The title compound was prepared as described in EXAMPLE 284D-E, substituting N-benzyl-2-[(1R)-5-bromo-2′,4′-dioxo-2,3-dihydro-3′H-spiro[indene-1,5′-[1,3]oxazolidin]-3′-yl]-N-[(1S)-1-cyclopropylethyl]acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide in EXAMPLE 284D. ¹H NMR (400 MHz, DMSO-d₆) δ ppm 7.46-7.33 (m, 2H), 7.33-7.01 (m, 3H), 6.53-6.40 (m, 2H), 5.43 (s, 2H), 4.77 (s, 1H), 4.74-4.52 (m, 1H), 4.52-4.21 (m, 1H), 3.77 (dq, J=9.6, 6.8 Hz, 1H), 3.11-2.79 (m, 2H), 2.40 (ddd, J=14.5, 8.0, 3.1 Hz, 1H), 1.56-0.67 (m, 7H), 0.59-0.10 (m, 4H); MS (ESI(+)) m/e 456 (M+Na)⁺.

### Example 287

### N-[(1S)-3′-(2-{[(1R)-1-cyclopropylethyl](4-fluorobenzyl)amino}-2-oxoethyl)-2′,4′-dioxo-2,3-dihydrospiro[indene-1,5′-[1,3]oxazolidin]-5-yl]-3,3-difluoroazetidine-1-carboxamide

### Example 287A

### 2-((S)-5-bromo-2′,4′-dioxo-2,3-dihydrospiro[indene-,5′-oxazolidin]-3′-yl)-N—((R)-1-cyclopropylethyl)-N-(4-fluorobenzyl)acetamide

The title compound was prepared as described in EXAMPLE 281F, substituting (S)-5-bromo-2,3-dihydrospiro[indene-1,5′-oxazolidine]-2′,4′-dione for (R)-5-bromo-2,3-dihydrospiro[indene-1,5′-oxazolidine]-2′,4′-dione and (R)-2-bromo-N-(1-cyclopropylethyl)-N-(4-fluorobenzyl)acetamide for (S)-2-bromo-N-(4-fluorobenzyl)-N-(1,1,1-trifluoropropan-2-yl)acetamide.

### Example 287B

### 2-((1S)-5-amino-2′,4′-dioxo-2,3-dihydrospiro[indene-,5′-oxazolidin]-3′-yl)-N—((R)-1-cyclopropylethyl)-N-(4-fluorobenzyl)acetamide

The title compound was prepared as described in EXAMPLE 284D-E, substituting 2-((S)-5-bromo-2′,4′-dioxo-2,3-dihydrospiro[indene-1,5′-oxazolidin]-3′-yl)-N—((R)-1-cyclopropylethyl)-N-(4-fluorobenzyl)acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide.

### Example 287C

### N-[(1S)-3′-(2-{[(1R)-1-cyclopropylethyl](4-fluorobenzyl)amino}-2-oxoethyl)-2′,4′-dioxo-2,3-dihydrospiro[indene-1,5′-[1,3]oxazolidin]-5-yl]-3,3-difluoroazetidine-1-carboxamide

The title compound was prepared as described in EXAMPLE 282, substituting 2-((1S)-5-amino-2′,4′-dioxo-2,3-dihydrospiro[indene-1,5′-oxazolidin]-3′-yl)-N—((R)-1-cyclopropylethyl)-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide. ¹H NMR (400 MHz, DMSO-d₆) δ ppm 9.03 (s, 1H), 7.61 (d, J=5.2 Hz, 1H), 7.53-7.02 (m, 6H), 4.76 (s, 1H), 4.63 (d, J=13.4 Hz, 1H), 4.51-4.17 (m, 5H), 3.82-3.68 (m, 1H), 3.38 (d, J=7.9 Hz, 1H), 3.14 (dd, J=16.2, 7.9 Hz, 1H), 3.02 (dq, J=12.3, 4.4, 3.7 Hz, 1H), 2.64 (ddd, J=12.5, 6.0, 2.8 Hz, 1H), 2.57-2.50 (m, 1H), 1.30-1.08 (m, 4H), 1.06-0.71 (m, 2H), 0.59-0.08 (m, 4H); MS (ESI(+)) m/e 593 (M+Na)⁺.

### Example 288

### N-[(1R)-1-cyclopropylethyl]-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 282, substituting 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆, Temp=90° C.) δ ppm 8.39 (s, 1H), 7.50 (d, J=1.8 Hz, 1H), 7.35 (d, J=12.7 Hz, 2H), 7.29-7.18 (m, 2H), 7.11 (s, 2H), 5.91 (t, J=4.7 Hz, 1H), 4.68 (s, 2H), 4.39 (s, 2H), 3.23-3.05 (m, 1H), 2.77-2.60 (m, 4H), 1.33-1.12 (m, 4H), 0.96 (dq, J=8.1, 4.2, 3.0 Hz, 2H), 0.86 (t, J=6.5 Hz, 2H), 0.50 (d, J=6.0 Hz, 1H), 0.25 (td, J=10.4, 9.4, 6.0 Hz, 3H); MS (ESI(+)) m/e 531.1 (M+Na)⁺.

### Example 289

### 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide

### Example 289A

### 5′-bromo-1′,3′-dihydrospiro[imidazolidine-4,2′-indene]-2,5-dione

A mixture of 5-bromo-1H-inden-2 (3H)-one (0.5 g, 2.369 mmol) in water (4.74 ml) and ethanol (4.74 mL) was treated with potassium cyanide (0.463 g, 7.11 mmol) followed by ammonium carbonate (1.366 g, 14.21 mmol). The reaction mixture was heated at 70° C. for 3 hours. After cooling to room temperature, the heterogeneous mixture was filtered. The filtrate was treated with 2 mL water and the resulting suspension was filtered. The tan solids were washed with water and dried under vacuum to provide the title compound which was used in the next step without purification.

### Example 289B

### 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide

The title compound was prepared as described in EXAMPLE 281F, substituting 5′-bromo-1′,3′-dihydrospiro[imidazolidine-4,2′-indene]-2,5-dione for (R)-5-bromo-2,3-dihydrospiro[indene-1,5′-oxazolidine]-2′,4′-dione and (S)-2-bromo-N-(1-cyclopropylethyl)-N-(4-fluorobenzyl)acetamide for (S)-2-bromo-N-(4-fluorobenzyl)-N-(1,1,1-trifluoropropan-2-yl)acetamide. ¹H NMR (400 MHz, DMSO-d₆, 90° C.) δ ppm 8.41 (brs, 1H), 7.43 (s, 1H), 7.40-7.25 (m, 3H), 7.18 (d, J=8.0 Hz, 1H), 7.14-7.00 (m, 2H), 4.62 (brs, 2H), 4.43-4.08 (m, 2H), 3.76-3.27 (m, 3H), 3.12 (dd, J=22.7, 16.9 Hz, 2H), 1.22-1.04 (m, 3H), 1.04-0.87 (m, 1H), 0.56-0.41 (m, 1H), 0.35-0.12 (m, 3H). MS (ESI⁺) m/z 516 (M+H)⁺.

### Example 290

### 2-(6-bromo-1,1-dioxido-2′,5′-dioxo-1′H-spiro[1-benzothiophene-3,4′-imidazolidin]-1′-yl)-N-[(1S)-1-cyclopropylethyl]-N-(4-fluorobenzyl)acetamide

### Example 290A

### 6-bromobenzo[b]thiophen-3(2H)-one 1,1-dioxide

To a solution of methyl 4-bromo-2-(methylsulfonyl)benzoate (5 g, 17.06 mmol) in tetrahydrofuran (64.6 ml) under nitrogen was added sodium hydride (0.771 g, 19.27 mmol) (60% in mineral oil) in five portions. The reaction mixture was stirred at room temperature for 20 hours and water was added followed by aqueous 1M HCl and ethyl acetate. The aqueous layer was extracted with ethyl acetate and the combined organic layers were washed with brine, dried over anhydrous sodium sulfate, filtered and concentrated. The solid was triturated with 2:1 hexanes/ether; the suspension was filtered and dried with MgSO₄ to give the title compound.

### Example 290B

### 6-bromo-2H-spiro[benzo[b]thiophene-3,4′-imidazolidine]-2′,5′-dione 1,1-dioxide

In a 50 mL high-pressure stainless steel vessel, a suspension of 6-bromobenzo[b]thiophen-3(2H)-one 1,1-dioxide (1 g, 3.83 mmol) in formamide (19.15 ml) was treated with potassium cyanide (0.748 g, 11.49 mmol) and the mixture was stirred for 5 minutes at which time a homogeneous solution had formed. Ammonium carbonate (2.208 g, 22.98 mmol) was added and the vessel was sealed. The reaction mixture was slowly heated up to 70° C. over 2 hours, and was heated at 110° C. for 10 hours. The reaction mixture was concentrated to dryness on a rotavap at 80° C. under high vacuum. The residue was treated with 120 mL 30% methanol/dichloromethane and the mixture was triturated, sonicated and stirred until a suspension had formed. The suspension was filtered through a bed of diatomaceous earth and the filtrate was concentrated. The concentrate was treated with 35 mL water and was extracted with ethyl acetate. The combined organic layers were dried over anhydrous sodium sulfate, filtered, and concentrated. The residue was purified by silica gel column chromatography and triturated with ether to afford the title compound.

### Example 290C

### 2-(6-bromo-1,1-dioxido-2′,5′-dioxo-1′H-spiro[1-benzothiophene-3,4′-imidazolidin]-1′yl)-N-[(1S)-1-cyclopropylethyl]-N-(4-fluorobenzyl)acetamide

6-Bromo-2H-spiro[benzo[b]thiophene-3,4′-imidazolidine]-2′,5′-dione 1,1-dioxide (0.03 g, 0.091 mmol) and (S)-2-bromo-N-(1-cyclopropylethyl)-N-(4-fluorobenzyl)acetamide (0.030 g, 0.095 mmol) in N,N-dimethylformamide (0.6 ml) was treated with N-ethyl-N-isopropylpropan-2-amine (0.040 ml, 0.226 mmol) and the reaction mixture was stirred at room temperature under nitrogen for 6 hours. Additional N-ethyl-N-isopropylpropan-2-amine (0.040 ml, 0.226 mmol) was added and the mixture was stirred at room temperature for 30 hours. The reaction mixture was quenched with saturated aqueous sodium chloride (5 mL) and extracted with ethyl acetate. The organic layer was dried over anhydrous sodium sulfate, filtered, and concentrated. The concentrate was purified by silica gel column chromatography to afford the title compound. ¹H NMR (400 MHz, DMSO-d₆, 90° C.) δ ppm 9.02 (brs, 1H), 8.08 (d, J=1.8 Hz, 1H), 8.00 (dd, J=8.5, 1.9 Hz, 1H), 7.68 (d, J=8.5 Hz, 1H), 7.44-7.21 (m, 2H), 7.21-7.00 (m, 2H), 4.66 (brs, 2H), 4.53-4.15 (m, 2H), 4.02 (d, J=14.5 Hz, 1H), 3.85 (d, J=14.5 Hz, 1H), 3.76-3.20 (m, 1H), 1.16 (d, J=6.3 Hz, 3H), 1.01-0.89 (m, 1H), 0.56-0.40 (m, 1H), 0.36-0.13 (m, 3H). MS (ESI⁺) m/z 566 (M+H)⁺.

### Example 291

### N-[(1R)-1-cyclopropylethyl]-N-(4-fluorobenzyl)-2-{(1S)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 282, substituting 2-((S)-5-amino-2′,4′-dioxo-2,3-dihydrospiro[indene-1,5′-oxazolidin]-3′-yl)-N—((R)-1-cyclopropylethyl)-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆, Temp=90° C.) δ ppm 8.39 (s, 1H), 7.50 (d, J=1.8 Hz, 1H), 7.36 (s, 2H), 7.29-7.18 (m, 2H), 7.11 (s, 2H), 5.92 (q, J=4.7 Hz, 1H), 4.68 (s, 2H), 3.12 (dt, J=15.5, 7.6 Hz, 1H), 3.04-2.99 (m, 1H), 1.32-1.08 (m, 5H), 0.97 (ddt, J=13.4, 8.4, 4.1 Hz, 1H), 0.90-0.73 (m, 2H), 0.51 (dp, J=9.0, 4.5 Hz, 1H), 0.27 (ddd, J=15.0, 9.6, 5.5 Hz, 3H); MS (ESI(+)) m/e 531 (M+Na)⁺.

### Example 292

### N-[(1R)-3′-(2-{[(1S)-1-cyclopropylethyl](4-fluorobenzyl)amino}-2-oxoethyl)-2′,4′-dioxo-2,3-dihydrospiro[indene-1,5′-[1,3]oxazolidin]-5-yl]-3,3-difluoroazetidine-1-carboxamide

### Example 292A

### 2-((R)-5-bromo-2′,4′-dioxo-2,3-dihydrospiro[indene-1,5′-oxazolidin]-3′-yl)-N—((S)-1-cyclopropylethyl)-N-(4-fluorobenzyl)acetamide

The title compound was prepared as described in EXAMPLE 281F, substituting (S)-2-bromo-N-(1-cyclopropylethyl)-N-(4-fluorobenzyl)acetamide for (S)-2-bromo-N-(4-fluorobenzyl)-N-(1,1,1-trifluoropropan-2-yl)acetamide.

### Example 292B

### 2-((R)-5-amino-2′,4′-dioxo-2,3-dihydrospiro[indene-1,5′-oxazolidin]-3′-yl)-N—((S)-1-cyclopropylethyl)-N-(4-fluorobenzyl)acetamide

The title compound was prepared as described in EXAMPLE 284D-E, substituting 2-((R)-5-bromo-2′,4′-dioxo-2,3-dihydrospiro[indene-1,5′-oxazolidin]-3′-yl)-N—((S)-1-cyclopropylethyl)-N-(4-fluorobenzyl)acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide.

### Example 292C

### N-[(1R)-3′-(2-{[(1S)-1-cyclopropylethyl](4-fluorobenzyl)amino}-2-oxoethyl)-2′,4′-dioxo-2,3-dihydrospiro[indene-1,5′-[1,3]oxazolidin]-5-yl]-3,3-difluoroazetidine-1-carboxamide

The title compound was prepared as described in EXAMPLE 282, substituting 2-((R)-5-amino-2′,4′-dioxo-2,3-dihydrospiro[indene-1,5′-oxazolidin]-3′-yl)-N—((S)-1-cyclopropylethyl)-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide. ¹H NMR (400 MHz, DMSO-d₆, Temp=90° C.) δ ppm 8.74 (s, 1H), 7.56 (d, J=1.8 Hz, 1H), 7.46-7.23 (m, 4H), 7.11 (s, 2H), 4.68 (s, 2H), 4.39 (s, 2H), 3.23-3.07 (m, 1H), 3.04 (dd, J=8.8, 4.2 Hz, 1H), 2.67 (ddd, J=14.9, 8.6, 6.5 Hz, 1H), 2.54-2.49 (m, 1H), 1.32-1.09 (m, 5H), 1.05-0.75 (m, 2H), 0.50 (d, J=8.7 Hz, 1H), 0.38-0.11 (m, 3H); MS (ESI(+)) m/e 593.1 (M+Na)⁺.

### Example 293

### N-[(1S)-3′-(2-{[(1S)-1-cyclopropylethyl](4-fluorobenzyl)amino}-2-oxoethyl)-2′,4′-dioxo-2,3-dihydrospiro[indene-1,5′-[1,3]oxazolidin]-5-yl]-3,3-difluoroazetidine-1-carboxamide

The title compound was prepared as described in EXAMPLE 282, substituting 2-[(1S)-5-amino-2′,4′-dioxo-2,3-dihydro-3′H-spiro[indene-1,5′-[1,3]oxazolidin]-3′-yl]-N-[(1S)-1-cyclopropylethyl]-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide. ¹H NMR (500 MHz, DMSO-d₆) δ ppm 9.03 (s, 1H), 7.61 (dd, J=6.9, 1.7 Hz, 1H), 7.54-7.39 (m, 1H), 7.39-7.17 (m, 4H), 7.17-7.00 (m, 1H), 4.76 (s, 1H), 4.71-4.52 (m, 1H), 3.20-2.95 (m, 2H), 2.75-2.50 (m, 2H), 1.31-1.10 (m, 6H), 1.04-0.73 (m, 5H), 0.57-0.09 (m, 4H); MS (ESI(+)) m/e 593 (M+Na)⁺.

### Example 294

### tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate

N-Benzyl-2-[(1R)-5-bromo-2′,4′-dioxo-2,3-dihydro-3′H-spiro[indene-1,5′-[1,3]oxazolidin]-3′-yl]-N-[(1S)-1-cyclopropylethyl]acetamide (1.2 g, 2.413 mmol), copper(I) iodide (0.060 g, 0.314 mmol) and PdCl₂(1,1′-bis(diphenylphosphino)ferrocene) (0.177 g, 0.241 mmol) were combined in a 4 mL vial with pressure relief cap and the vessel was degassed. Dimethylacetamide (12.06 ml) was added, the solution was degassed for 45 minutes and was treated with a solution of (1-(tert-butoxycarbonyl)azetidin-3-yl)zinc(II) iodide (5.17 ml, 3.62 mmol). The mixture was heated at 85° C. for 3 hours, cooled to room temperature, and quenched with water. The separated aqueous layer was extracted with ethyl acetate and the combined organic layers were dried (Na₂SO₄), filtered and concentrated. Silica gel column chromatography gave the title compound. ¹H NMR (400 MHz, DMSO-d₆, T=90° C.) δ 7.45 (d, J=7.9 Hz, 1H), 7.42-7.28 (m, 5H), 7.25 (dd, J=7.9, 1.7 Hz, 2H), 4.71 (s, 2H), 4.58-4.30 (m, 2H), 4.30-4.19 (m, 2H), 3.89-3.79 (m, 3H), 3.23-3.13 (m, 1H), 3.09 (dd, J=8.9, 4.6 Hz, 1H), 2.75-2.64 (m, 1H), 2.56-2.46 (m, 1H), 1.41 (s, 9H), 1.19-1.17 (m, 3H), 1.04-0.92 (m, 1H), 0.55-0.46 (m, 1H), 0.33-0.21 (m, 3H). MS (ESI⁺) m/z 596 (M+H)⁺.

### Example 295

### N-[(1S)-1-cyclopropylethyl]-2-[2,5-dioxo-5′-(6-oxo-1,6-dihydropyridin-3-yl)-1′,3′-dihydro-1H-spiro[imidazolidine-4,2′-inden]-1-yl]-N-(4-fluorobenzyl)acetamide

In a 4 mL vial, a mixture of 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide (0.06 g, 0.117 mmol), 5-(4,4,5,5-tetramethyl-1,3,2-dioxaborolan-2-yl)pyridin-2-ol (0.032 g, 0.146 mmol), potassium carbonate (0.049 g, 0.351 mmol), and PdCl₂(1,1′-bis(diphenylphosphino)ferrocene)-dichloromethane adduct (7.62 mg, 9.33 μmol) in degassed dioxane (1 ml) and water (0.2 mL) was sealed under nitrogen and was heated at 95° C. for 7 hours. The reaction mixture was cooled to room temperature, diluted with ethyl acetate and filtered through a bed of diatomaceous earth. The filtrate was concentrated and the residue was purified by reverse-phase HPLC to give the title compound. ¹H NMR (400 MHz, DMSO-d₆, 90° C.) δ ppm 8.42 (brs, 1H), 7.74 (dd, J=9.5, 2.8 Hz, 1H), 7.60 (d, J=2.7 Hz, 1H), 7.42-7.28 (m, 4H), 7.25 (d, J=7.9 Hz, 1H), 7.16-7.01 (m, 2H), 6.41 (d, J=9.4 Hz, 1H), 4.63 (brs, 2H), 4.29-4.11 (m, 2H), 3.64-3.10 (m, 5H), 1.15 (d, J=6.7 Hz, 3H), 1.01-0.89 (m, 1H), 0.55-0.43 (m, 1H), 0.31-0.13 (m, 3H). MS (ESI⁺) m/z 529 (M+H)⁺.

### Example 296

### 2-(5′-amino-2,5-dioxo-1′,3′-dihydro-1H-spiro[imidazolidine-4,2′-inden]-1-yl)-N-[(1S)-1-cyclopropylethyl]-N-(4-fluorobenzyl)acetamide

### Example 296A

### N—((S)-1-cyclopropylethyl)-2-(5′-((diphenylmethylene)amino)-2,5-dioxo-1′,3′-dihydrospiro[imidazolidine-4,2′-inden]-1-yl)-N-(4-fluorobenzyl)acetamide

Pd₂(dibenzylideneacetone)₃ (0.021 g, 0.023 mmol) and (2,2′-bis(diphenylphosphino)-1,1′-binaphthyl) (0.029 g, 0.047 mmol) were placed in a 4 mL vial under nitrogen. 2-(5′-Bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide (0.12 g, 0.233 mmol), sodium 2-methylpropan-2-olate (0.067 g, 0.700 mmol), diphenylmethanimine (0.055 ml, 0.327 mmol) and degassed toluene (1.6 ml) were added and the reaction mixture was heated under nitrogen at 90° C. for 3.5 hours. The reaction mixture was cooled to room temperature, diluted with 50 mL ethyl acetate and filtered through a bed of diatomaceous earth. The filtrate was washed with brine, dried over anhydrous sodium sulfate and concentrated. The concentrate was purified by silica gel column chromatography to give the title compound.

### Example 296B

### 2-(5′-amino-2,5-dioxo-1′,3′-dihydro-1H-spiro[imidazolidine-4,2′-inden]-1-yl)-N-[(1S)-1-cyclopropylethyl]-N-(4-fluorobenzyl)acetamide

A solution of N—((S)-1-cyclopropylethyl)-2-(5′-((diphenylmethylene)amino)-2,5-dioxo-1′,3′-dihydrospiro[imidazolidine-4,2′-inden]-1-yl)-N-(4-fluorobenzyl)acetamide (0.072 g, 0.117 mmol) in tetrahydrofuran (1.463 ml) was treated with aqueous 2M HCl (0.585 ml, 1.170 mmol) and the reaction mixture was stirred at room temperature overnight. The reaction mixture was concentrated to dryness and the residue was triturated with ether. The ether supernatant was removed using a syringe and discarded. The residual solid was partitioned in saturated aqueous sodium bicarbonate and ethyl acetate. The organic layer was dried over anhydrous sodium sulfate, filtered, and concentrated. The concentrate was purified by silica gel column chromatography followed by reverse-phase HPLC to provide the title compound. ¹H NMR (400 MHz, DMSO-d₆, 90° C.) δ ppm 8.38 (s, 1H), 7.34 (d, J=9.4 Hz, 2H), 7.07 (d, J=8.0 Hz, 3H), 6.83-6.72 (m, 2H), 4.62 (brs, 2H), 4.34-4.08 (m, 2H), 3.45-3.15 (m, 3H), 3.05 (dd, J=16.5, 9.8 Hz, 2H), 1.15 (d, J=6.8 Hz, 3H), 1.02-0.88 (m, 1H), 0.58-0.38 (m, 1H), 0.35-0.12 (m, 3H). MS (ESI⁺) m/z 451 (M+H)⁺.

### Example 297

### 2-[(1R)-5-(6-aminopyridin-3-yl)-2′,4′-dioxo-2,3-dihydro-3′H-spiro[indene-1,5′-[1,3]oxazolidin]-3′-yl]-N-[(1S)-1-cyclopropylethyl]-N-(4-fluorobenzyl)acetamide

The title compound was prepared as described in EXAMPLE 295, substituting 2-((R)-5-bromo-2′,4′-dioxo-2,3-dihydrospiro[indene-1,5′-oxazolidin]-3′-yl)-N—((S)-1-cyclopropylethyl)-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 5-(4,4,5,5-tetramethyl-1,3,2-dioxaborolan-2-yl)pyridin-2-amine for 5-(4,4,5,5-tetramethyl-1,3,2-dioxaborolan-2-yl)pyridin-2-ol. ¹H NMR (400 MHz, DMSO-d₆, 90° C.) δ ppm 8.25 (d, J=2.5 Hz, 1H), 7.70 (dd, J=8.6, 2.6 Hz, 1H), 7.55 (s, 1H), 7.48 (d, J=2.5 Hz, 2H), 7.37 (brs, 2H), 7.12 (brs, 2H), 6.57 (d, J=8.6 Hz, 1H), 6.07-5.65 (m, 2H), 4.69 (brs, 2H), 4.61-4.26 (m, 2H), 3.80-3.31 (m, 1H), 3.30-3.06 (m, 2H), 2.77-2.66 (m, 1H), 2.60-2.48 (m, 1H), 1.19 (d, J=6.7 Hz, 3H), 1.04-0.92 (m, 1H), 0.58-0.44 (m, 1H), 0.38-0.20 (m, 3H). MS (ESI⁺) m/z 529 (M+H)⁺.

### Example 298

### 2-{(1R)-5-[1-(2-amino-2-oxoethyl)-1H-pyrazol-4-yl]-2′,4′-dioxo-2,3-dihydro-3′H-spiro[indene-1,5′-[1,3]oxazolidin]-3′-yl}-N-[(1S)-1-cyclopropylethyl]-N-(4-fluorobenzyl)acetamide

The title compound was prepared as described in EXAMPLE 295, substituting 2-((R)-5-bromo-2′,4′-dioxo-2,3-dihydrospiro[indene-1,5′-oxazolidin]-3′-yl)-N—((S)-1-cyclopropylethyl)-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆, 90° C.) δ ppm 8.11 (s, 1H), 7.86 (s, 1H), 7.57 (s, 1H), 7.54-7.47 (m, 1H), 7.47-7.26 (m, 3H), 7.22-6.90 (m, 4H), 4.76 (s, 2H), 4.69 (brs, 2H), 4.63-4.22 (m, 2H), 3.85-3.31 (m, 1H), 3.28-3.03 (m, 2H), 2.77-2.63 (m, 1H), 2.63-2.49 (m, 1H), 1.19 (d, J=6.7 Hz, 3H), 1.06-0.91 (m, 1H), 0.61-0.43 (m, 1H), 0.43-0.20 (m, 3H). MS (ESI⁺) m/z 560.2 (M+H)⁺.

### Example 299

### N-[(1S)-1-cyclopropylethyl]-2-[(1R)-2′,4′-dioxo-5-(pyridin-3-yl)-2,3-dihydro-3′H-spiro[indene-1,5′-[1,3]oxazolidin]-3′-yl]-N-(4-fluorobenzyl)acetamide

The title compound was prepared as described in EXAMPLE 295, substituting 2-((R)-5-bromo-2′,4′-dioxo-2,3-dihydrospiro[indene-1,5′-oxazolidin]-3′-yl)-N—((S)-1-cyclopropylethyl)-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and pyridin-3-ylboronic acid for 5-(4,4,5,5-tetramethyl-1,3,2-dioxaborolan-2-yl)pyridin-2-ol. ¹H NMR (400 MHz, DMSO-d₆, 90° C.) δ ppm 8.87 (d, J=2.4 Hz, 1H), 8.58 (dd, J=4.8, 1.6 Hz, 1H), 8.10-7.99 (m, 1H), 7.72 (d, J=1.6 Hz, 1H), 7.65 (dd, J=8.1, 1.7 Hz, 1H), 7.58 (d, J=8.0 Hz, 1H), 7.46 (dd, J=8.0, 4.8 Hz, 1H), 7.43-7.26 (m, 2H), 7.26-7.04 (m, 2H), 4.69 (brs, 2H), 4.64-4.24 (m, 2H), 3.88-3.33 (m, 1H), 3.33-3.08 (m, 2H), 2.83-2.67 (m, 1H), 2.65-2.53 (m, 1H), 1.19 (d, J=6.7 Hz, 3H), 1.07-0.90 (m, 1H), 0.61-0.42 (m, 1H), 0.41-0.19 (m, 3H). MS (ESI⁺) m/z 514 (M+H)⁺.

### Example 300

### N-[(1S)-1-cyclopropylethyl]-2-[1,1-dioxido-2′,5′-dioxo-6-(6-oxo-1,6-dihydropyridin-3-yl)-1′H-spiro[1-benzothiophene-3,4′-imidazolidin]-1′-yl]-N-(4-fluorobenzyl)acetamide

The title compound was prepared as described in EXAMPLE 295, substituting 2-(6-bromo-1,1-dioxido-2′,5′-dioxo-1′H-spiro[1-benzothiophene-3,4′-imidazolidin]-1′-yl)-N-[(1S)-1H-cyclopropylethyl]-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide. ¹H NMR (400 MHz, DMSO-d₆, 90° C.) δ ppm 8.04-7.94 (m, 2H), 7.88 (d, J=8.2 Hz, 2H), 7.74 (dd, J=8.8, 1.8 Hz, 1H), 7.49-7.23 (m, 2H), 7.23-6.95 (m, 2H), 6.48-6.39 (m, 1H), 4.66 (brs, 2H), 4.54-4.10 (m, 2H), 3.99 (d, J=14.5 Hz, 1H), 3.82 (d, J=14.5 Hz, 1H), 3.77-3.23 (m, 1H), 1.17 (d, J=6.7 Hz, 3H), 1.02-0.89 (m, 1H), 0.58-0.43 (m, 1H), 0.38-0.12 (m, 3H). MS (ESI⁺) m/z 579 (M+H)⁺.

### Example 301

### N-benzyl-2-(7-bromo-2′,4′-dioxo-2,3-dihydro-3′H-spiro[chromene-4,5′-[1,3]oxazolidin]-3′-yl)-N-[(1S)-1-cyclopropylethyl]acetamide

### Example 301A

### 7-bromo-4-((trimethylsilyl)oxy)chroman-4-carbonitrile

A mixture of 7-bromochroman-4-one (3 g, 13.21 mmol) in ether (6 ml) was treated with trimethylsilanecarbonitrile (1.991 ml, 15.86 mmol) followed by zinc iodide (0.084 g, 0.264 mmol) and the reaction mixture was stirred at room temperature for 17 hours. The mixture was diluted with ether (15 mL), treated with activated carbon (30 mg), and filtered through a bed of diatomaceous earth. The filtrate was concentrated and purified by silica gel column chromatography to give the title compound.

### Example 301B

### ethyl 7-bromo-4-hydroxychroman-4-carbimidate

A solution of 7-bromo-4-((trimethylsilyl)oxy)chroman-4-carbonitrile (4.4 g, 13.49 mmol) in ethanol (38.5 ml) was cooled in an ice bath and perfused with hydrogen chloride gas for 30 minutes. The reaction mixture (homogeneous solution) was stirred at 0° C. for an additional 1.5 hours and then concentrated under vacuum. The residue was triturated with ether and filtered to afford the title compound.

### Example 301C

### 7-bromo-4′-ethoxy-2′H-spiro[chroman-4,5′-oxazol]-2′-one

A suspension of ethyl 7-bromo-4-hydroxychroman-4-carbimidate hydrochloride (1.12 g, 3.33 mmol) in tetrahydrofuran (13 ml) was treated with triethylamine (1.391 ml, 9.98 mmol) and the mixture was cooled in an ice bath. Triphosgene (0.454 g, 1.531 mmol) was added portionwise over 3 minutes and the reaction mixture was stirred at room temperature for 16 hours. The reaction mixture was cooled in an ice bath and 6 mL aqueous 2M HCl was cautiously added. The quenched reaction mixture was stirred at room temperature for 20 minutes and diluted with ethyl acetate. The aqueous layer was extracted with ethyl acetate and the combined organic layers were washed with brine, dried over anhydrous sodium sulfate, filtered and concentrated to give the title compound. [Note: Partial or complete formation of oxazolidine dione at this step was occasionally observed with this or other substrates thus obviating the need for acid or base catalyzed hydrolysis of the intermediate 4-ethoxyoxazolone as in EXAMPLE 301D.]

### Example 301D

### 7-bromospiro[chroman-4,5′-oxazolidine]-2′,4′-dione

7-Bromo-4′-ethoxy-2′H-spiro[chroman-4,5′-oxazol]-2′-one (1 g, 3.07 mmol) in tetrahydrofuran (10.95 ml) and water (4.38 ml) was treated with potassium carbonate (0.509 g, 3.68 mmol) and the mixture was stirred at room temperature overnight. The reaction mixture was partitioned between ethyl acetate and brine. The aqueous layer was extracted with ethyl acetate and the combined organic layers were washed with aqueous 1 M HCl and brine, dried over anhydrous sodium sulfate, filtered, and concentrated. Purification by silica gel column chromatography provided the title compound.

### Example 301E

### N-benzyl-2-(7-bromo-2′,4′-dioxo-2,3-dihydro-3′H-spiro[chromene-4,5′-[1,3]oxazolidin]-3′-yl)-N-[(1S)-1-cyclopropylethyl]acetamide

The title compound was prepared as described in EXAMPLE 281F, substituting 7-bromospiro[chroman-4,5′-oxazolidine]-2′,4′-dione for (R)-5-bromo-2,3-dihydrospiro[indene-1,5′-oxazolidine]-2′,4′-dione and (S)—N-benzyl-2-bromo-N-(1-cyclopropylethyl)acetamide for (S)-2-bromo-N-(4-fluorobenzyl)-N-(1,1,1-trifluoropropan-2-yl)acetamide. ¹H NMR (400 MHz, DMSO-d₆, 90° C.) δ ppm 7.42-7.19 (m, 6H), 7.19-7.10 (m, 2H), 4.71 (brs, 2H), 4.61-4.28 (m, 3H), 4.28-4.13 (m, 1H), 3.82-3.37 (m, 1H), 2.48-2.40 (m, 2H), 1.17 (dd, J=6.6, 2.8 Hz, 3H), 1.05-0.91 (m, 1H), 0.57-0.43 (m, 1H), 0.35-0.16 (m, 3H). MS (ESI⁺) m/z 515 (M+H)⁺.

### Example 302

### 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide

tert-Butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate (700 mg, 1.220 mmol) was dissolved in dichloromethane (5 ml) and trifluoroacetic acid (5 ml) was added. The mixture was stirred for 2 hours at room temperature and concentrated to dryness. The crude product was redissolved in dichloromethane, treated with 1 mL 3M HCl in cyclopentyl methyl ether and concentrated. The material was dissolved and reconcentrated from diethyl ether to afford the title compound as a hydrochloride salt. ¹H NMR (400 MHz, DMSO-d₆, T=90° C.) δ 9.50-8.79 (m, 2H), 7.49 (d, J=7.9 Hz, 1H), 7.44 (s, 1H), 7.41-7.18 (m, 6H), 4.71 (s, 2H), 4.60-4.25 (m, 4H), 4.18 (p, J=7.7 Hz, 1H), 4.10 (d, J=9.2 Hz, 2H), 3.24-3.14 (m, 1H), 3.13-3.03 (m, 1H), 2.71 (ddd, J=14.9, 8.6, 6.4 Hz, 1H), 2.58-2.48 (m, 1H), 1.18 (d, J=6.2 Hz, 3H), 0.98 (ddt, J=13.5, 8.5, 4.2 Hz, 1H), 0.55-0.45 (m, 1H), 0.36-0.18 (m, 3H); MS (ESI⁺) m/z 474 (M+H)⁺.

### Example 303

### 2-[(1R)-5-(1-acetylazetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide

To a suspension of 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide (50 mg, 0.098 mmol) in dichloromethane (1 mL) was added triethylamine (0.041 mL, 0.29 mmol) followed by acetic anhydride (11 mg, 0.11 mmol). The reaction was stirred overnight at room temperature, and was partitioned between water and dichloromethane. The aqueous layer was extracted with dichloromethane and the combined organic layers were dried (Na₂SO₄), filtered and concentrated. Silica gel column chromatography provided the title compound. ¹H NMR (400 MHz, DMSO-d₆, T=90° C.) δ 7.45 (d, J=8.0 Hz, 1H), 7.38 (s, 1H), 7.37-7.19 (m, 6H), 4.71 (s, 2H), 4.59-3.98 (m, 5H), 3.87 (tt, J=9.0, 5.8 Hz, 2H), 3.18 (dt, J=15.3, 7.3 Hz, 1H), 3.07 (ddd, J=16.5, 8.7, 4.4 Hz, 1H), 2.69 (ddd, J=14.9, 8.7, 6.5 Hz, 1H), 2.58-2.46 (m, 1H), 1.78 (s, 3H), 1.17 (d, J=5.3 Hz, 3H), 0.98 (dtt, J=10.1, 7.1, 3.5 Hz, 1H), 0.55-0.46 (m, 1H), 0.40-0.16 (m, 3H); MS (ESI⁺) m/z 516 (M+H)⁺.

### Example 304

### N-benzyl-N-[(1S)-1-cyclopropylethyl]-2-[(1R)-5-(1-methylazetidin-3-yl)-2′,4′-dioxo-2,3-dihydro-3′H-spiro[indene-1,5′-[1,3]oxazolidin]-3′-yl]acetamide

To a suspension of 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide (50 mg, 0.098 mmol) in dichloromethane (1 mL) was added formaldehyde (37% solution in water, 0.029 mL, 0.39 mmol). The mixture was stirred at room temperature for 1 hour and then acetic acid (0.007 mL, 0.12 mmol) and sodium triacetoxyborohydride (25 mg, 0.12 mmol) were added. The resulting mixture was stirred overnight at room temperature and was diluted with saturated aqueous NaHCO₃. The mixture was stirred for 1 hour and extracted with dichloromethane. The aqueous layer was extracted with dichloromethane and the combined organic layers were dried (Na₂SO₄), filtered and concentrated. Silica gel column chromatography provided the title compound. ¹H NMR (400 MHz, DMSO-d₆, T=90° C.) δ 7.39 (d, J=7.9 Hz, 1H), 7.37-7.19 (m, 7H), 4.70 (s, 2H), 4.55-4.25 (m, 2H), 3.67-3.57 (m, 3H), 3.22-3.11 (m, 3H), 3.09-2.97 (m, 1H), 2.68 (ddd, J=14.8, 8.6, 6.4 Hz, 1H), 2.56-2.44 (m, 1H), 2.28 (s, 3H), 1.17 (d, J=6.6 Hz, 3H), 0.98 (ddt, J=13.5, 8.4, 4.2 Hz, 1H), 0.58-0.42 (m, 1H), 0.41-0.13 (m, 3H); MS (ESI⁺) m/z 488 (M+H)⁺.

### Example 305

### 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]-N-methylazetidine-1-carboxamide

The title compound was prepared as described in EXAMPLE 282, substituting 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆, T=90° C.) δ 7.43 (d, J=7.9 Hz, 1H), 7.40-7.18 (m, 7H), 5.97-5.89 (m, 1H), 4.70 (s, 2H), 4.62-4.28 (m, 2H), 4.24-4.13 (m, 2H), 3.87-3.71 (m, 3H), 3.18 (dt, J=15.2, 7.4 Hz, 1H), 3.12-3.01 (m, 1H), 2.69 (ddd, J=15.0, 8.4, 6.5 Hz, 1H), 2.58 (d, J=4.7 Hz, 3H), 2.56-2.45 (m, 1H), 1.19-1.14 (m, 3H), 1.04-0.93 (m, 1H), 0.50 (s, 1H), 0.38-0.15 (m, 3H); MS (ESI⁺) m/z 531 (M+H)⁺.

### Example 306

### N-benzyl-2-{(1R)-5-[1-(cyclopropylcarbonyl)azetidin-3-yl]-2′,4′-dioxo-2,3-dihydro-3′H-spiro[indene-1,5′-[1,3]oxazolidin]-3′-yl}-N-[(1 S)-1-cyclopropylethyl]acetamide

2-[(1R)-5-(Azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide hydrochloride, N-(3-dimethylaminopropyl)-N′-ethylcarbodiimide hydrochloride (16.74 mg, 0.108 mmol) and 1-hydroxybenzotriazole hydrate (13.25 mg, 0.098 mmol) were taken up in dichloromethane (1 ml). To the mixture was added triethylamine (0.041 ml, 0.294 mmol) followed by cyclopropanecarboxylic acid (7.77 μl, 0.098 mmol). The mixture was stirred overnight at room temperature and partitioned between water and dichloromethane. The aqueous layer was extracted with dichloromethane and the combined organic layers were dried (Na₂SO₄), filtered and concentrated. Silica gel column chromatography provided the title compound. ¹H NMR (400 MHz, DMSO-d₆, T=90° C.) δ 7.45 (d, J=8.0 Hz, 1H), 7.42-7.14 (m, 7H), 4.71 (s, 2H), 4.62-4.23 (m, 5H), 4.15-3.98 (m, 1H), 3.93 (td, J=8.7, 4.3 Hz, 1H), 3.19 (dt, J=15.4, 7.4 Hz, 1H), 3.08 (ddd, J=16.4, 8.6, 4.3 Hz, 1H), 2.70 (ddd, J=15.0, 8.6, 6.6 Hz, 1H), 2.58-2.47 (m, 1H), 1.55 (tt, J=7.7, 5.1 Hz, 1H), 1.22-1.13 (m, 3H), 0.98 (ddt, J=13.3, 8.2, 4.1 Hz, 1H), 0.89-0.80 (m, 1H), 0.76-0.67 (m, 4H), 0.50 (s, 1H), 0.32-0.20 (m, 3H); MS (ESI⁺) m/z 542 (M+H)⁺.

### Example 307

### N-benzyl-N-[(1S)-1-cyclopropylethyl]-2-{(1R)-5-[1-(2-hydroxy-2-methylpropanoyl)azetidin-3-yl]-2′,4′-dioxo-2,3-dihydro-3′H-spiro[indene-1,5′-[1,3]oxazolidin]-3′-yl}acetamide

The title compound was prepared as described in EXAMPLE 306, substituting 2-hydroxybutyric acid for cyclopropanecarboxylic acid. ¹H NMR (400 MHz, DMSO-d₆, T=90° C.) δ 7.45 (d, J=7.9 Hz, 1H), 7.40-7.16 (m, 7H), 4.74 (s, 1H), 4.71 (s, 2H), 4.38 (s, 3H), 3.86 (tt, J=8.7, 6.0 Hz, 1H), 3.19 (dt, J=15.3, 7.4 Hz, 1H), 3.07 (ddd, J=16.6, 8.6, 4.4 Hz, 1H), 2.69 (ddd, J 5=14.9, 8.7, 6.5 Hz, 1H), 2.57-2.48 (m, 1H), 1.29 (s, 6H), 1.21-1.12 (m, 3H), 0.98 (ddt, J=13.6, 8.5, 4.2 Hz, 1H), 0.85 (q, J=6.8, 6.4 Hz, 2H), 0.50 (s, 1H), 0.37-0.17 (m, 3H); MS (ESI⁺) m/z 560 (M+H)⁺.

### Example 308

### N-benzyl-N-[(1S)-1-cyclopropylethyl]-2-{(1R)-5-[1-(methylsulfonyl)azetidin-3-yl]-2′,4′-dioxo-2,3-dihydro-3′H-spiro[indene-1,5′-[1,3]oxazolidin]-3′-yl}acetamide

2-[(1R)-5-(Azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide hydrochloride (50 mg, 0.098 mmol) was dissolved in dichloromethane (1 ml) and to the mixture was added triethylamine (0.041 ml, 0.294 mmol) and methanesulfonyl chloride (7.98 μl, 0.103 mmol). The mixture was stirred overnight at room temperature and partitioned between water and dichloromethane. The aqueous layer was extracted with dichloromethane and the combined organic layers were dried (Na₂SO₄), filtered and concentrated. Silica gel column chromatography purification gave the title compound. ¹H NMR (400 MHz, DMSO-d₆, T=90° C.) δ 7.46 (d, J=7.9 Hz, 1H), 7.40 (s, 1H), 7.39-7.18 (m, 6H), 4.71 (s, 2H), 4.65-4.28 (m, 2H), 4.26-4.17 (m, 2H), 3.93 (dt, J=8.2, 3.7 Hz, 3H), 3.19 (dt, J=15.3, 7.4 Hz, 1H), 3.08 (ddd, J=16.3, 8.6, 4.4 Hz, 1H), 2.98 (s, 3H), 2.70 (ddd, J=14.9, 8.7, 6.5 Hz, 1H), 2.57-2.48 (m, 1H), 1.22-1.13 (m, 3H), 0.98 (ddt, J=13.6, 8.5, 4.3 Hz, 1H), 0.89-0.80 (m, 1H), 0.50 (s, 1H), 0.27 (td, J=8.6, 5.0 Hz, 2H); MS (ESI⁺) m/z 552 (M+H)⁺.

### Example 309

### N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]oxazolidin]-5-yl]cyclobutanecarboxamide

### Example 309A

### 2-[(1R)-5-amino-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 284D-E, substituting 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide.

### Example 309B

### N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]oxazolidin]-5-yl]cyclobutanecarboxamide

The title compound was prepared as described in EXAMPLE 306, substituting cyclobutyl carboxylic acid for cyclopropanecarboxylic acid and 2-[(1R)-5-amino-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆) δ ppm 7.69 (d, J=1.8 Hz, 1H), 7.45 (dd, J=8.4, 1.9 Hz, 1H), 7.39-7.27 (m, 3H), 7.15 (t, J=8.5 Hz, 2H), 5.33-5.06 (m, 1H), 4.88-4.78 (m, 1H), 4.72-4.29 (m, 2H), 3.24-3.11 (m, 2H), 3.03 (ddd, J=16.5, 8.6, 4.2 Hz, 1H), 2.68 (ddd, J=15.0, 8.6, 6.5 Hz, 1H), 2.24 (dt, J=10.7, 8.5 Hz, 2H), 2.19-2.11 (m, 2H), 1.95 (dq, J=11.1, 8.5 Hz, 1H), 1.85 (tt, J=10.2, 4.3 Hz, 1H), 1.37 (d, J=6.9 Hz, 3H); MS (APCI⁺) m/z 562 (M+H)⁺.

TABLE 14
The following EXAMPLEs were prepared essentially as described in EXAMPLE 309B,
substituting cyclobutyl carboxylic acid with an appropriate carboxylic acid. Some products were
purified by silica gel column chromatography while others were purified by reverse-phase HPLC.
Accordingly, some EXAMPLEs were isolated as trifluoroacetic acid salts.

| Ex | Name | NMR | MS |
|---|---|---|---|
| 310 | 3-ethoxy-N-[(1R)-3′-(2-{(4- | ¹H NMR (400 MHz, DMSO-d₆) δ ppm | (APCI⁺) |
| | fluorobenzyl)[(2S)-1,1,1- | 7.68 (d, J = 1.8 Hz, 1H), 7.43 (dd, J = 8.3, 1.9 Hz, | m/z 580 |
| | trifluoropropan-2-yl]amino}-2- | 1H), 7.37-7.30 (m, 3H), 7.15 (t, J = 8.5 Hz, | (M + H)⁺ |
| | oxoethyl)-2′,4′-dioxo-2,3- | 2H), 5.20 (s, 1H), 4.83 (d, J = 17.6 Hz, 1H), | |
| | dihydrospiro[indene-1,5′- | 4.71-4.30 (m, 2H), 3.69 (t, J = 6.3 Hz, 2H), | |
| | [1,3]oxazolidin]-5- | 3.46 (q, J = 7.0 Hz, 2H), 3.21-3.12 (m, 1H), | |
| | yl]propanamide | 3.04 (ddd, J = 16.4, 8.7, 4.2 Hz, 1H), | |
| | | 2.69 (ddd, J = 15.0, 8.6, 6.4 Hz, 1H), 2.56 (t, J = 6.3 Hz, | |
| | | 3H), 1.37 (d, J = 7.0 Hz, 3H), 1.10 (t, | |
| | | J = 7.0 Hz, 3H) | |
| 311 | N-[(1R)-3′-(2-{(4- | ¹H NMR (400 MHz, DMSO-d₆) δ 10.21 (s, | (ESI(+)) |
| | fluorobenzyl)[(2S)-1,1,1- | 1H), 7.88 (d, J = 3.2 Hz, 1H), 7.79 (d, J = 4.1 Hz, | m/e 591 |
| | trifluoropropan-2-yl]amino}-2- | 1H), 7.52-7.31 (m, 3H), | (M + H)⁺ |
| | oxoethyl)-2′,4′-dioxo-2,3- | 7.30-6.97 (m, 3H), 5.24 (dp, J = 135.7, 7.5 Hz, 1H), | |
| | dihydrospiro[indene-1,5′- | 5.03-4.14 (m, 5H), 3.23-2.94 (m, 2H), | |
| | [1,3]oxazolidin]-5-yl]-5-oxo-D- | 2.81-2.45 (m, 2H), 2.44-2.09 (m, 3H), | |
| | prolinamide | 2.06-1.94 (m, 1H), 1.37 (dd, J = 17.0, 6.9 Hz, | |
| | | 3H) | |
| 312 | N-[(1R)-3′-(2-{(4- | ¹H NMR (400 MHz, DMSO-d₆) δ 10.21 (s, | (ESI(+)) |
| | fluorobenzyl)[(2S)-1,1,1- | 1H), 7.88 (d, J = 3.2 Hz, 1H), 7.77 (d, J = 5.4 Hz, | m/e 591 |
| | trifluoropropan-2-yl]amino}-2- | 1H), 7.57-7.32 (m, 3H), | (M + H)⁺ |
| | oxoethyl)-2′,4′-dioxo-2,3- | 7.31-6.92 (m, 3H), 5.24 (dp, J = 135.9, 7.6 Hz, 1H), | |
| | dihydrospiro[indene-1,5′- | 5.03-4.16 (m, 5H), 3.27-2.94 (m, 2H), | |
| | [1,3]oxazolidin]-5-yl]-5-oxo-L- | 2.71-2.50 (m, 2H), 2.42-2.08 (m, 3H), | |
| | prolinamide | 1.99 (ddt, J = 12.3, 8.6, 4.8 Hz, 1H), | |
| | | 1.36 (dd, J = 16.7, 6.9 Hz, 3H) | |
| 313 | N-[(1R)-3′-(2-{(4- | ¹H NMR (400 MHz, DMSO-d₆) δ ppm | (APCI⁺) |
| | fluorobenzyl)[(2S)-1,1,1- | 7.67 (d, J = 1.8 Hz, 1H), 7.44 (dd, J = 8.3, 1.9 Hz, | m/z 536 |
| | trifluoropropan-2-yl]amino}-2- | 1H), 7.32 (t, J = 8.5 Hz, 3H), 7.15 (t, J = 8.4 Hz, | (M + H)⁺ |
| | oxoethyl)-2′,4′-dioxo-2,3- | 2H), 5.37-5.01 (m, 1H), 4.83 (d, J = 17.6 Hz, | |
| | dihydrospiro[indene-1,5′- | 1H), 4.74-4.49 (m, 2H), | |
| | [1,3]oxazolidin]-5- | 4.45-4.28 (m, 1H), 3.16 (dt, J = 15.4, 7.7 Hz, 1H), | |
| | yl]propanamide | 3.03 (ddd, J = 16.5, 8.7, 4.3 Hz, 1H), | |
| | | 2.68 (ddd, J = 15.0, 8.7, 6.5 Hz, 1H), 2.34 (q, J = 7.5 Hz, | |
| | | 2H), 1.37 (d, J = 7.0 Hz, 3H), 1.11 (t, | |
| | | J = 7.6 Hz, 3H) | |
| 314 | N-[(1R)-3′-(2-{(4- | ¹H NMR (400 MHz, DMSO-d₆) δ ppm | (APCI⁺) |
| | fluorobenzyl)[(2S)-1,1,1- | 7.67 (d, J = 1.8 Hz, 1H), 7.44 (dd, J = 8.4, 1.9 Hz, | m/z 548 |
| | trifluoropropan-2-yl]amino}-2- | 1H), 7.32 (t, J = 8.2 Hz, 3H), 7.15 (t, J = 8.6 Hz, | (M + H)⁺ |
| | oxoethyl)-2′,4′-dioxo-2,3- | 2H), 5.40-5.02 (m, 1H), 4.83 (d, J = 17.7 Hz, | |
| | dihydrospiro[indene-1,5′- | 1H), 4.74-4.51 (m, 2H), | |
| | [1,3]oxazolidin]-5- | 4.45-4.28 (m, 1H), 3.15 (dt, J = 15.3, 7.4 Hz, 1H), | |
| | yl]cyclopropanecarboxamide | 3.03 (ddd, J = 16.4, 8.7, 4.3 Hz, 1H), | |
| | | 2.68 (ddd, J = 15.0, 8.6, 6.4 Hz, 1H), 1.79 (tt, J = 7.8, | |
| | | 4.7 Hz, 1H), 1.37 (d, J = 7.0 Hz, 3H), | |
| | | 0.88-0.77 (m, 5H) | |
| 315 | N-(4-fluorobenzyl)-2-{(1R)-5- | ¹H NMR (400 MHz, DMSO-d₆) δ ppm | (APCI⁺) |
| | [(methoxyacetyl)amino]-2′,4′- | 7.72 (d, J = 1.8 Hz, 1H), 7.51 (dd, J = 8.2, 2.0 Hz, | m/z 552 |
| | dioxo-2,3-dihydro-3′H- | 1H), 7.40-7.29 (m, 3H), 7.15 (t, J = 8.5 Hz, | (M + H)⁺ |
| | spiro[indene-1,5′- | 2H), 5.34-5.02 (m, 1H), 4.84 (d, J = 17.7 Hz, | |
| | [1,3]oxazolidin]-3′-yl}-N-[(2S)- | 1H), 4.74-4.52 (m, 2H), | |
| | 1,1,1-trifluoropropan-2- | 4.46-4.24 (m, 1H), 4.00 (s, 2H), 3.40 (s, 3H), 3.17 (dt, | |
| | yl]acetamide | J = 15.4, 7.4 Hz, 1H), 3.05 (ddd, J = 16.5, | |
| | | 8.7, 4.3 Hz, 1H), 2.69 (ddd, J = 14.9, 8.7, 6.5 Hz, | |
| | | 1H), 1.37 (d, J = 7.0 Hz, 3H) | |
| 316 | 2-{(1R)-5- | ¹H NMR (400 MHz, DMSO-d₆) δ ppm | (APCI⁺) |
| | [(ethoxyacetyl)amino]-2′,4′- | 7.71 (d, J = 1.8 Hz, 1H), 7.51 (dd, J = 8.5, 1.9 Hz, | m/z 566 |
| | dioxo-2,3-dihydro-3′H- | 1H), 7.34 (dd, J = 10.0, 5.6 Hz, 3H), 7.15 (t, | (M + H)⁺ |
| | spiro[indene-1,5′- | J = 8.8 Hz, 2H), 5.37-5.02 (m, 1H), 4.84 (d, | |
| | [1,3]oxazolidin]-3′-yl}-N-(4- | J = 17.5 Hz, 1H), 4.74-4.52 (m, 2H), | |
| | fluorobenzyl)-N-[(2S)-1,1,1- | 4.48-4.31 (m, 1H), 4.03 (s, 2H), 3.60 (q, J = 7.0 Hz, | |
| | trifluoropropan-2-yl]acetamide | 2H), 3.17 (dt, J = 15.4, 7.5 Hz, 1H), | |
| | | 3.05 (ddd, J = 16.5, 8.6, 4.2 Hz, 1H), 2.69 (ddd, J = 14.9, | |
| | | 8.6, 6.4 Hz, 1H), 1.38 (d, J = 7.0 Hz, | |
| | | 3H), 1.20 (t, J = 7.0 Hz, 3H) | |
| 317 | 3,3,3-trifluoro-N-[(1R)-3′-(2- | ¹H NMR (400 MHz, DMSO-d₆) δ ppm | (APCI⁺) |
| | {(4-fluorobenzyl)[(2S)-1,1,1- | 7.66 (d, J = 1.8 Hz, 1H), 7.42 (dd, J = 8.3, 2.0 Hz, | m/z 590 |
| | trifluoropropan-2-yl]amino}-2- | 1H), 7.39-7.28 (m, 3H), 7.15 (t, J = 8.3 Hz, | (M + H)⁺ |
| | oxoethyl)-2′,4′-dioxo-2,3- | 2H), 5.42-5.04 (m, 1H), 4.84 (d, J = 17.5 Hz, | |
| | dihydrospiro[indene-1,5′- | 1H), 4.73-4.51 (m, 2H), | |
| | [1,3]oxazolidin]-5- | 4.45-4.30 (m, 1H), 3.45 (q, J = 11.0 Hz, 2H), 3.18 (dt, | |
| | yl]propanamide | J = 15.3, 7.4 Hz, 1H), 3.06 (ddd, J = 16.4, | |
| | | 8.6, 4.3 Hz, 1H), 2.70 (ddd, J = 14.9, 8.6, 6.5 Hz, | |
| | | 1H), 1.38 (d, J = 7.0 Hz, 3H) | |
| 318 | N-[(1R)-3′-(2-{(4- | ¹H NMR (400 MHz, DMSO-d₆) δ ppm | (APCI⁺) |
| | fluorobenzyl)[(2S)-1,1,1- | 7.69 (d, J = 1.8 Hz, 1H), 7.43 (dd, J = 8.4, 1.9 Hz, | m/z 564 |
| | trifluoropropan-2-yl]amino}-2- | 1H), 7.38-7.27 (m, 3H), 7.15 (t, J = 8.4 Hz, | (M + H)⁺ |
| | oxoethyl)-2′,4′-dioxo-2,3- | 2H), 5.39-5.03 (m, 1H), 4.83 (d, J = 17.5 Hz, | |
| | dihydrospiro[indene-1,5′- | 1H), 4.74-4.47 (m, 2H), | |
| | [1,3]oxazolidin]-5-yl]-3- | 4.45-4.25 (m, 1H), 3.16 (dt, J = 15.4, 7.5 Hz, 1H), | |
| | methylbutanamide | 3.04 (ddd, J = 16.5, 8.7, 4.2 Hz, 1H), 2.68 (ddd, J = 15.0, | |
| | | 8.6, 6.4 Hz, 1H), 2.21 (d, J = 7.0 Hz, | |
| | | 2H), 2.09 (dp, J = 13.6, 6.7 Hz, 1H), 1.37 (d, | |
| | | J = 7.0 Hz, 3H), 0.95 (d, J = 6.6 Hz, 7H) | |
| 319 | N-[(1R)-3′-(2-{(4- | ¹H NMR (400 MHz, DMSO-d₆) δ ppm | (APCI⁺) |
| | fluorobenzyl)[(2S)-1,1,1- | 7.69 (d, J = 1.8 Hz, 1H), 7.45 (dd, J = 8.3, 1.9 Hz, | m/z 550 |
| | trifluoropropan-2-yl]amino}-2- | 1H), 7.37-7.26 (m, 3H), 7.15 (t, J = 8.7 Hz, | (M + H)⁺ |
| | oxoethyl)-2′,4′-dioxo-2,3- | 2H), 5.37-4.96 (m, 1H), 4.83 (d, J = 17.6 Hz, | |
| | dihydrospiro[indene-1,5′- | 1H), 4.74-4.49 (m, 2H), | |
| | [1,3]oxazolidin]-5-yl]-2- | 4.46-4.27 (m, 1H), 3.16 (dt, J = 15.4, 7.5 Hz, 1H), | |
| | methylpropanamide | 3.03 (ddd, J = 16.5, 8.7, 4.2 Hz, 1H), | |
| | | 2.74-2.57 (m, 2H), 1.37 (d, J = 7.0 Hz, 3H), 1.12 (d, J = 6.8 Hz, | |
| | | 7H) | |
| 320 | 2-[(1R)-5-(acetylamino)-2′,4′- | ¹H NMR (400 MHz, DMSO-d₆) δ ppm | (APCI⁺) |
| | dioxo-2,3-dihydro-3′H- | 7.66 (d, J = 1.8 Hz, 1H), 7.42 (dd, J = 8.4, 1.9 Hz, | m/z 522 |
| | spiro[indene-1,5′- | 1H), 7.33 (t, J = 7.9 Hz, 3H), 7.15 (t, J = 8.5 Hz, | (M + H)⁺ |
| | [1,3]oxazolidin]-3′-yl]-N-(4- | 2H), 5.21 (s, 1H), 4.84 (d, J = 17.6 Hz, | |
| | fluorobenzyl)-N-[(2S)-1,1,1- | 1H), 4.63 (s, 2H), 4.38 (s, 1H), 3.16 (dt, J = 15.4, | |
| | trifluoropropan-2-yl]acetamide | 7.4 Hz, 1H), 3.04 (ddd, J = 16.6, 8.7, | |
| | | 4.3 Hz, 1H), 2.68 (ddd, J = 14.8, 8.6, 6.5 Hz, | |
| | | 1H), 2.06 (s, 3H), 1.37 (d, J = 7.0 Hz, 3H) | |
| 401 | N-[(1R)-3′-(2-{(4- | ¹H NMR (400 MHz, DMSO-d₆) δ 10.40 (s, | (ESI(+)) |
| | fluorobenzyl)[(2S)-1,1,1- | 1H), 8.02-7.87 (m, 3H), 7.70-7.06 (m, | m/e 606 |
| | trifluoropropan-2-yl]amino}-2- | 9H), 5.52-4.12 (m, 5H), 3.26-2.98 (m, | (M + Na)⁺ |
| | oxoethyl)-2′,4′-dioxo-2,3- | 2H), 2.76-2.52 (m, 2H), 1.37 (dd, J = 17.7, | |
| | dihydrospiro[indene-1,5′- | 6.9 Hz, 3H) | |
| | [1,3]oxazolidin]-5-yl]benzamide | | |
| 402 | N-[(1R)-3′-(2-{(4- | ¹H NMR (400 MHz, DMSO-d₆) δ 9.94 (s, | (ESI(+)) |
| | fluorobenzyl)[(2S)-1,1,1- | 1H), 8.32 (s, 1H), 8.02 (d, J = 2.6 Hz, 1H), | m/e 610 |
| | trifluoropropan-2-yl]amino}-2- | 7.84 (s, 1H), 7.57 (t, J = 9.6 Hz, 1H), | (M + Na)⁺ |
| | oxoethyl)-2′,4′-dioxo-2,3- | 7.45-7.08 (m, 5H), 5.52-4.11 (m, 5H), | |
| | dihydrospiro[indene-1,5′- | 3.26-2.95 (m, 2H), 2.75-2.54 (m, 2H), 1.37 (dd, J = 17.3, | |
| | [1,3]oxazolidin]-5-yl]-1-methyl- | 6.9 Hz, 3H) | |
| | 1H-pyrazole-4-carboxamide | | |
| 403 | N-[(1R)-3′-(2-{(4- | ¹H NMR (400 MHz, DMSO-d₆) δ 10.16 (s, | (ESI(+)) |
| | fluorobenzyl)[(2S)-1,1,1- | 1H), 7.95 (s, 1H), 7.85 (d, J = 2.2 Hz, 1H), | m/e 610 |
| | trifluoropropan-2-yl]amino}-2- | 7.68 (t, J = 9.0 Hz, 1H), 7.44-7.09 (m, 5H), | (M + Na)⁺ |
| | oxoethyl)-2′,4′-dioxo-2,3- | 6.77 (t, J = 2.3 Hz, 1H), 5.51-4.16 (m, 5H), | |
| | dihydrospiro[indene-1,5′- | 3.17 (dt, J = 15.0, 7.5 Hz, 1H), | |
| | [1,3]oxazolidin]-5-yl]-1-methyl- | 3.11-2.97 (m, 1H), 2.75-2.61 (m, 1H), 2.61-2.51 (m, | |
| | 1H-pyrazole-3-carboxamide | 1H), 1.36 (dd, J = 18.1, 6.9 Hz, 3H) | |
| 404 | N-[(1R)-3′-(2-{(4- | ¹H NMR (400 MHz, DMSO-d₆) δ 10.64 (s, | (ESI⁻) |
| | fluorobenzyl)[(2S)-1,1,1- | 1H), 8.89-8.70 (m, 2H), 7.87 (dd, J = 9.9, | m/z 539 |
| | trifluoropropan-2-yl]amino}-2- | 8.4 Hz, 3H), 7.64 (t, J = 9.3 Hz, 1H), | (M − CO₂H)⁻ |
| | oxoethyl)-2′,4′-dioxo-2,3- | 7.47-7.12 (m, 5H), 5.53-4.15 (m, 5H), | |
| | dihydrospiro[indene-1,5′- | 3.20-3.01 (m, 2H), 2.75-2.52 (m, 2H), 1.37 (dd, J = 17.3, | |
| | [1,3]oxazolidin]-5-yl]pyridine-4- | 6.9 Hz, 3H) | |
| | carboxamide | | |
| 405 | N-[(1R)-3′-(2-{(4- | ¹H NMR (400 MHz, DMSO-d₆) δ 10.78 (s, | (ESI(+)) |
| | fluorobenzyl)[(2S)-1,1,1- | 1H), 8.76 (d, J = 4.1 Hz, 1H), 8.26-7.98 (m, | m/e 607 |
| | trifluoropropan-2-yl]amino}-2- | 3H), 7.85-7.64 (m, 2H), 7.49-7.08 (m, | (M + Na)⁺ |
| | oxoethyl)-2′,4′-dioxo-2,3- | 5H), 5.54-4.13 (m, 5H), 3.23-3.02 (m, | |
| | dihydrospiro[indene-1,5′- | 2H), 2.76-2.54 (m, 2H), 1.37 (dd, J = 17.9, | |
| | [1,3]oxazolidin]-5-yl]pyridine-2- | 6.9 Hz, 3H) | |
| | carboxamide | | |
| 502 | N-[(1R)-3′-(2-{(4- | ¹H NMR (400 MHz, DMSO-d₆) δ 10.75 (d, J = 6.4 Hz, | (ESI(+)) |
| | fluorobenzyl)[(2S)-1,1,1- | 1H), 9.47 (s, 1H), 8.70 (s, 1H), | m/e 577 |
| | trifluoropropan-2-yl]amino}-2- | 7.86-6.96 (m, 7H), 5.24 (dp, J = 129.6, 7.5 Hz, | (M + H)⁺ |
| | oxoethyl)-2′,4′-dioxo-2,3- | 1H), 5.06-4.13 (m, 5H), | |
| | dihydrospiro[indene-1,5′- | 3.40-2.97 (m, 5H), 2.67 (dh, J = 15.0, 7.5 Hz, 1H), | |
| | [1,3]oxazolidin]-5- | 2.40 (dt, J = 14.5, 6.1 Hz, 1H), 1.97 (dh, J = 14.4, | |
| | yl]prolinamide | 7.8 Hz, 3H), 1.36 (dd, J = 15.5, 6.9 Hz, 3H). | |
| 504 | N-[(1R)-3′-(2-{(4- | ¹H NMR (400 MHz, DMSO-d₆) δ 10.19 (s, | (ESI(+)) |
| | fluorobenzyl)[(2S)-1,1,1- | 1H), 8.08-7.09 (m, 8H), 6.83 (s, 1H), | m/e 596 |
| | trifluoropropan-2-yl]amino}-2- | 5.25 (dp, J = 140.4, 7.4 Hz, 1H), 5.05-4.14 (m, | (M + Na)⁺ |
| | oxoethyl)-2′,4′-dioxo-2,3- | 4H), 3.24-2.97 (m, 2H), 2.89-2.53 (m, | |
| | dihydrospiro[indene-1,5′- | 2H), 1.37 (dd, J = 18.4, 6.9 Hz, 3H). | |
| | [1,3]oxazolidin]-5-yl]-1H- | | |
| | pyrazole-5-carboxamide | | |
| 506 | N-(4-fluorobenzyl)-2-[(1R)-5- | ¹H NMR (501 MHz, DMSO-d₆) δ 10.48 (d, J = 2.8 Hz, | (ESI(+)) |
| | {[(methylsulfamoyl)acetyl]amino}- | 1H), 7.77 (d, J = 4.5 Hz, 1H), | m/e 632 |
| | 2′,4′-dioxo-2,3-dihydro-3′H- | 7.55-7.02 (m, 7H), 5.41 (p, J = 7.7 Hz, 1H), | (M + NH₄)⁺ |
| | spiro[indene-1,5′- | 5.04-4.18 (m, 4H), 4.14 (d, J = 3.1 Hz, 2H), | |
| | [1,3]oxazolidin]-3′-yl]-N-[(2S)- | 3.17-3.01 (m, 2H), 2.65-2.50 (m, 5H), 1.36 (dd, | |
| | 1,1,1-trifluoropropan-2- | J = 20.8, 7.0 Hz, 3H). | |
| | yl]acetamide | | |
| 507 | N-(4-fluorobenzyl)-2-[(1R)-5- | ¹H NMR (501 MHz, DMSO-d₆) δ 10.59 (d, J = 3.1 Hz, | (ESI(+)) |
| | {[(methylsulfonyl)acetyl]amino}- | 1H), 7.77 (d, J = 5.2 Hz, 1H), | m/e 617 |
| | 2′,4′-dioxo-2,3-dihydro-3′H- | 7.50-6.77 (m, 6H), 5.41 (p, J = 7.7 Hz, 1H), | (M + NH₄)⁺ |
| | spiro[indene-1,5′- | 5.03-4.13 (m, 6H), 3.22-3.00 (m, 5H), | |
| | [1,3]oxazolidin]-3′-yl]-N-[(2S)- | 2.76-2.51 (m, 2H), 1.36 (dd, J = 21.0, 6.9 Hz, 3H). | |
| | 1,1,1-trifluoropropan-2- | | |
| | yl]acetamide | | |
| 508 | 2-[(1R)-5- | ¹H NMR (400 MHz, DMSO-d₆) δ 10.55 (s, | (ESI(+)) |
| | {[(dimethylsulfamoyl)acetyl]amino}- | 1H), 7.76 (d, J = 3.6 Hz, 1H), 7.51-7.00 (m, | m/e 645 |
| | 2′,4′-dioxo-2,3-dihydro-3′H- | 6H), 5.50-5.04 (m, 1H), 5.03-4.22 (m, | (M + NH₄)⁺ |
| | spiro[indene-1,5′- | 4H), 4.19 (s, 2H), 3.26-2.95 (m, 2H), | |
| | [1,3]oxazolidin]-3′-yl]-N-(4- | 2.85 (d, J = 2.3 Hz, 6H), 2.77-2.50 (m, 2H), | |
| | fluorobenzyl)-N-[(2S)-1,1,1- | 1.36 (dd, J = 17.0, 6.9 Hz, 3H). | |
| | trifluoropropan-2-yl]acetamide | | |
| 509 | 2-{(1R)-2′,4′-dioxo-5- | ¹H NMR (400 MHz, DMSO-d₆) δ 10.42 (s, | (ESI(+)) |
| | [(sulfamoylacetyl)amino]-2,3- | 1H), 7.77 (d, J = 4.1 Hz, 1H), 7.60-6.84 (m, | m/e 618 |
| | dihydro-3′H-spiro[indene-1,5′- | 8H), 5.50-5.01 (m, 1H), 5.07-4.18 (m, | (M + NH₄)⁺ |
| | [1,3]oxazolidin]-3′-yl}-N-(4- | 4H), 4.10 (s, 2H), 3.30-2.98 (m, 2H), | |
| | fluorobenzyl)-N-[(2S)-1,1,1- | 2.77-2.53 (m, 2H), 1.36 (dd, J = 16.8, 6.9 Hz, | |
| | trifluoropropan-2-yl]acetamide | 3H). | |
| 573 | (2R)-3,3,3-trifluoro-N-[(1R)-3′- | ¹H NMR (400 MHz, DMSO-d₆) δ 10.08 (s, | (ESI(+)) |
| | (2-{(4-fluorobenzyl)[(2S)-1,1,1- | 1H), 7.86 (d, J = 2.3 Hz, 1H), 7.72-6.97 (m, | m/e 620 |
| | trifluoropropan-2-yl]amino}-2- | 7H), 5.59-4.02 (m, 5H), 3.23-2.93 (m, | (M + H)⁺ |
| | oxoethyl)-2′,4′-dioxo-2,3- | 2H), 2.76-2.31 (m, 2H), 1.58 (s, 3H), | |
| | dihydrospiro[indene-1,5′- | 1.36 (dd, J = 18.0, 6.9 Hz, 3H) | |
| | [1,3]oxazolidin]-5-yl]-2- | | |
| | hydroxy-2-methylpropanamide | | |
| 574 | (2S)-3,3,3-trifluoro-N-[(1R)-3′- | ¹H NMR (400 MHz, DMSO-d₆) δ 10.09 (s, | (ESI(+)) |
| | (2-{(4-fluorobenzyl)[(2S)-1,1,1- | 1H), 7.87 (s, 1H), 7.70-7.01 (m, 7H), | m/e 620 |
| | trifluoropropan-2-yl]amino}-2- | 5.49-4.04 (m, 5H), 3.26-2.95 (m, 2H), | (M + H)⁺ |
| | oxoethyl)-2′,4′-dioxo-2,3- | 2.78-2.43 (m, 2H), 1.58 (s, 3H), 1.36 (dd, J = 17.8, | |
| | dihydrospiro[indene-1,5′- | 6.9 Hz, 3H) | |
| | [1,3]oxazolidin]-5-yl]-2- | | |
| | hydroxy-2-methylpropanamide | | |

### Example 321

### 2-(7-amino-2′,4′-dioxo-2,3-dihydro-3′H-spiro[chromene-4,5′-[1,3]oxazolidin]-3′-yl)-N-benzyl-N-[(1S)-1-cyclopropylethyl]acetamide

The title compound was prepared as described in EXAMPLE 284D-E, substituting N-benzyl-2-(7-bromo-2′,4′-dioxo-2,3-dihydro-3′H-spiro[chromene-4,5′-[1,3]oxazolidin]-3′-yl)-N-[(1S)-1-cyclopropylethyl]acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide. H NMR (400 MHz, DMSO-d₆, 90° C.) δ ppm 0.13-0.36 (m, 3H), 0.41-0.57 (m, 1H), 0.89-1.04 (m, 1H), 1.10-1.21 (m, 3H), 2.21-2.41 (m, 2H), 3.19-3.83 (m, 1H), 3.98-4.10 (m, 1H), 4.21-4.48 (m, 3H), 4.68 (s, 2H), 5.15 (s, 2H), 6.01-6.08 (m, 1H), 6.18 (dd, J=8.4, 2.3 Hz, 1H), 6.98 (dd, J=8.4, 1.6 Hz, 1H), 7.03-7.41 (m, 5H). MS (ESI⁺) m/z 472 (M+Na)⁺.

### Example 322

### 2-(6-amino-1,1-dioxido-2′,5′-dioxo-1′H-spiro[1-benzothiophene-3,4′-imidazolidin]-1′-yl)-N-[(1S)-1-cyclopropylethyl]-N-(4-fluorobenzyl)acetamide

The title compound was prepared as described in EXAMPLE 296A-B, substituting 2-(6-bromo-1,1-dioxido-2′,5′-dioxo-1′H-spiro[1-benzothiophene-3,4′-imidazolidin]-1′-yl)-N-[(1S)-1-cyclopropylethyl]-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide. ¹H NMR (400 MHz, DMSO-d₆, 90° C.) δ ppm 0.16-0.36 (m, 3H), 0.45-0.53 (m, 1H), 0.88-1.02 (m, 1H), 1.11-1.21 (m, 3H), 3.16-3.72 (m, 2H), 3.83 (d, J=14.2 Hz, 1H), 4.12-4.50 (m, 2H), 4.65 (s, 2H), 5.72 (s, 2H), 6.76-6.85 (m, 1H), 6.87-6.98 (m, 1H), 7.01-7.23 (m, 2H), 7.23-7.47 (m, 3H), 8.83 (s, 1H). MS (ESI⁺) m/z 501 (M+H)⁺.

### Example 323

### N-[(1S)-1-cyclopropylethyl]-N-(4-fluorobenzyl)-2-{5′-[(methylcarbamoyl)amino]-2,5-dioxo-1′,3′-dihydro-1H-spiro[imidazolidine-4,2′-inden]-1-yl}acetamide

The title compound was prepared as described in EXAMPLE 282, substituting 2-(5′-amino-2,5-dioxo-1′,3′-dihydro-1H-spiro[imidazolidine-4,2′-inden]-1-yl)-N-[(1S)-1-cyclopropylethyl]-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine for 3,3-difluoroazetidine hydrochloride. ¹H NMR (400 MHz, DMSO-d₆, 90° C.) δ ppm 8.39 (brs, 1H), 8.14 (brs, 1H), 7.47-7.27 (m, 3H), 7.20-6.99 (m, 4H), 5.90-5.77 (m, 1H), 4.62 (brs, 2H), 4.37-4.09 (m, 2H), 3.79-3.37 (m, 1H), 3.32 (dd, J=16.5, 9.7 Hz, 2H), 3.13-3.00 (m, 2H), 2.64 (d, J=4.5 Hz, 3H), 1.19-1.10 (m, 3H), 1.01-0.88 (m, 1H), 0.59-0.42 (m, 1H), 0.37-0.17 (m, 3H). MS (ESI⁺) m/z 530 (M+Na)⁺.

### Example 324

### N-(4-fluorobenzyl)-2-[(1R)-5-{[(2-methoxyethyl)(methyl)carbamoyl]amino}-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 282, substituting 2-[(1R)-5-amino-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and 2-methoxy-N-methylethanamine for 3,3-difluoroazetidine hydrochloride. ¹H NMR (400 MHz, DMSO-d₆) δ ppm 7.50 (s, 1H), 7.32 (dt, J=9.2, 2.0 Hz, 3H), 7.24 (dd, J=8.3, 3.1 Hz, 1H), 7.15 (t, J=8.8 Hz, 2H), 5.36-5.02 (m, 1H), 4.88-4.78 (m, 1H), 4.75-4.53 (m, 2H), 4.46-4.26 (m, 1H), 3.50 (dq, J=9.2, 4.9 Hz, 5H), 3.31 (s, 3H), 3.14 (dt, J=15.4, 7.5 Hz, 1H), 3.02 (td, J=8.2, 7.6, 4.3 Hz, 1H), 2.97 (s, 3H), 2.67 (ddd, J=15.0, 8.8, 6.6 Hz, 1H), 1.37 (d, J=7.0 Hz, 3H); MS (APCI⁺⁾ m/z 595 (M+H)⁺.

TABLE 15
The following EXAMPLEs were prepared essentially as described in EXAMPLE 324, substituting
an appropriate amine for 2-methoxy-N-methylethanamine. Some products were purified
by silica gel column chromatography while others were purified by reverse-phase HPLC.
Accordingly, some EXAMPLEs were isolated as trifluoroacetic acid salts.

| Ex | Name | | MS |
|---|---|---|---|
| 325 | 2-{(1R)-5- | ¹H NMR (400 MHz, DMSO-d₆) δ ppm 7.52 (d, J = | (APCI⁺) |
| | [(dimethylcarbamoyl)amino]- | 1.8 Hz, 1H), 7.39-7.29 (m, 3H), 7.24 (dd, J = 8.4, | m/z 551 |
| | 2′,4′-dioxo-2,3- | 3.0 Hz, 1H), 7.15 (t, J = 7.9 Hz, 3H), 5.34-5.02 | (M + H)⁺ |
| | dihydro-3′H- | (m, 1H), 4.83 (d, J = 17.4 Hz, 1H), 4.73-4.52 (m, | |
| | spiro[indene-1,5′- | 3H), 4.44-4.30 (m, 1H), 3.13 (dt, J = 15.4, 7.2 | |
| | [1,3]oxazolidin]-3′-yl}- | Hz, 1H), 3.01 (ddd, J = 16.5, 8.6, 4.1 Hz, 1H), 2.93 | |
| | N-(4-fluorobenzyl)-N- | (s, 6H), 2.67 (ddd, J = 15.0, 8.7, 6.6 Hz, 1H), 2.50- | |
| | [(2S)-1,1,1- | 2.45 (m, 1H), 1.37 (d, J = 7.0 Hz, 3H) | |
| | trifluoropropan-2- | | |
| | yl]acetamide | | |
| 326 | N-(4-fluorobenzyl)-2- | ¹H NMR (400 MHz, DMSO-d₆) δ ppm 7.52 (d, J = | (APCI⁺) |
| | [(1R)-5-{[methyl(2- | 1.6 Hz, 1H), 7.38-7.30 (m, 3H), 7.24 (dd, J = 8.4, | m/z 593 |
| | methylpropyl)carbam- | 2.8 Hz, 1H), 7.15 (t, J = 7.7 Hz, 2H), 5.36-4.95 | (M + H)⁺ |
| | oyl]amino}-2′,4′-dioxo- | (m, 1H), 4.83 (d, J = 17.5 Hz, 1H), 4.73-4.52 (m, | |
| | 2,3-dihydro-3′H- | 2H), 4.45-4.28 (m, 1H), 3.16 (d, J = 7.3 Hz, 2H), | |
| | spiro[indene-1,5′- | 3.11 (t, J = 7.7 Hz, 1H), 3.01 (ddd, J = 16.2, 8.7, | |
| | [1,3]oxazolidin]-3′-yl]- | 4.1 Hz, 1H), 2.95 (s, 3H), 2.67 (ddd, J = 15.0, 8.6, | |
| | N-[(2S)-1,1,1- | 6.5 Hz, 1H), 2.50-2.45 (m, 1H), 1.94 (dp, J = | |
| | trifluoropropan-2- | 13.7, 6.9 Hz, 1H), 1.37 (d, J = 7.0 Hz, 3H), 0.87 (d, | |
| | yl]acetamide | J = 6.7 Hz, 7H) | |
| 327 | 2-[(1R)-5- | ¹H NMR (400 MHz, DMSO-d₆) δ ppm 7.49 (s, | (APCI⁺) |
| | {[(cyclopropylmethyl)car- | 1H), 7.38-7.30 (m, 2H), 7.25-7.21 (m, 2H), 7.15 | m/z 577 |
| | bamoyl]amino}-2′,4′- | (t, J = 8.7 Hz, 2H), 5.33-4.96 (m, 1H), 4.83 (d, J = | (M + H)⁺ |
| | dioxo-2,3-dihydro-3′H- | 17.7 Hz, 1H), 4.74-4.50 (m, 2H), 4.45-4.28 | |
| | spiro[indene-1,5′- | (m, 1H), 3.13 (dt, J = 15.5, 7.5 Hz, 1H), 3.06- | |
| | [1,3]oxazolidin]-3′-yl]- | 2.95 (m, 4H), 2.67 (ddd, J = 15.0, 8.7, 6.6 Hz, 1H), | |
| | N-(4-fluorobenzyl)-N- | 2.50-2.44 (m, 1H), 1.37 (d, J = 7.0 Hz, 3H), 1.00- | |
| | [(2S)-1,1,1- | 0.91 (m, 1H), 0.46-0.41 (m, 2H), 0.21-0.16 | |
| | trifluoropropan-2- | (m, 2H) | |
| | yl]acetamide | | |
| 328 | 2-{(1R)-5- | ¹H NMR (400 MHz, DMSO-d₆) δ ppm 7.47 (d, J = | (APCI⁺) |
| | [(cyclobutylcarbamoyl) | 1.7 Hz, 1H), 7.38-7.30 (m, 2H), 7.27-7.21 (m, | m/z 577 |
| | amino]-2′,4′-dioxo-2,3- | 2H), 7.15 (t, J = 8.8 Hz, 2H), 5.34-5.01 (m, 1H), | (M + H)⁺ |
| | dihydro-3′H- | 4.83 (d, J = 17.6 Hz, 1H), 4.74-4.48 (m, 2H), | |
| | spiro[indene-1,5′- | 4.45-4.28 (m, 1H), 4.11 (p, J = 8.1 Hz, 1H), 3.13 | |
| | [1,3]oxazolidin]-3′-yl}- | (dt, J = 15.5, 7.5 Hz, 1H), 3.00 (ddd, J = 16.4, 8.8, | |
| | N-(4-fluorobenzyl)-N- | 4.1 Hz, 1H), 2.66 (ddd, J = 15.0, 8.6, 6.5 Hz, 1H), | |
| | [(2S)-1,1,1- | 2.50-2.44 (m, 1H), 2.23 (ddt, J = 10.8, 7.6, 3.8 | |
| | trifluoropropan-2- | Hz, 2H), 1.87 (pd, J = 9.2, 2.8 Hz, 2H), 1.72-1.56 | |
| | yl]acetamide | (m, 2H), 1.37 (d, J = 7.0 Hz, 3H) | |
| 329 | N-(4-fluorobenzyl)-2- | ¹H NMR (400 MHz, DMSO-d₆) δ ppm 7.50 (d, J = | (APCI⁺) |
| | [(1R)-5-({[(2R)-1- | 1.7 Hz, 1H), 7.37-7.28 (m, 2H), 7.27-7.19 (m, | m/z 609 |
| | hydroxy-3- | 2H), 7.15 (t, J = 8.7 Hz, 2H), 5.35-5.02 (m, 1H), | (M + H)⁺ |
| | methylbutan-2- | 4.83 (d, J = 17.6 Hz, 1H), 4.72-4.51 (m, 2H), | |
| | yl]carbamoyl}amino)- | 4.45-4.28 (m, 1H), 3.53-3.41 (m, 3H), 3.13 (dt, | |
| | 2′,4′-dioxo-2,3-dihydro- | J = 15.5, 7.5 Hz, 1H), 3.00 (ddd, J = 16.4, 8.7, 4.1 | |
| | 3′H-spiro[indene-1,5′- | Hz, 1H), 2.67 (ddd, J = 15.0, 8.8, 6.6 Hz, 1H), 2.50- | |
| | [1,3]oxazolidin]-3′-yl]- | 2.44 (m, 1H), 1.92-1.81 (m, 1H), 1.37 (d, J = | |
| | N-[(2S)-1,1,1- | 7.0 Hz, 3H), 0.90 (dd, J = 10.9, 6.8 Hz, 6H) | |
| | trifluoropropan-2- | | |
| | yl]acetamide | | |
| 330 | 2-[(1R)-2′,4′-dioxo-5- | ¹H NMR (400 MHz, DMSO-d₆) δ ppm 7.48 (d, J = | (APCI⁺) |
| | ({[(2S)- | 1.6 Hz, 1H), 7.37-7.29 (m, 2H), 7.27-7.20 (m, | m/z 607 |
| | tetrahydrofuran-2- | 2H), 7.15 (t, J = 8.7 Hz, 2H), 5.36-4.99 (m, 1H), | (M + H)⁺ |
| | ylmethyl]carbamoyl}amino)- | 4.83 (d, J = 17.5 Hz, 1H), 4.73-4.52 (m, 2H), | |
| | 2,3-dihydro-3′H- | 4.46-4.30 (m, 1H), 3.88 (qd, J = 6.7, 4.6 Hz, 1H), | |
| | spiro[indene-1,5′- | 3.79 (dt, J = 8.2, 6.5 Hz, 1H), 3.69-3.61 (m, 1H), | |
| | [1,3]oxazolidin]-3′-yl]- | 3.22 (d, J = 4.6 Hz, 1H), 3.19-3.08 (m, 2H), 3.01 | |
| | N-(4-fluorobenzyl)-N- | (ddd, J = 16.5, 8.7, 4.1 Hz, 1H), 2.67 (ddd, J = | |
| | [(2S)-1,1,1- | 15.0, 8.8, 6.6 Hz, 1H), 2.50-2.44 (m, 1H), 1.96- | |
| | trifluoropropan-2- | 1.78 (m, 3H), 1.55 (ddd, J = 10.8, 8.1, 5.5 Hz, 1H), | |
| | yl]acetamide | 1.37 (d, J = 7.0 Hz, 3H) | |
| 331 | 2-[(1R)-2′,4′-dioxo-5- | ¹H NMR (400 MHz, DMSO-d₆) δ ppm 7.48 (d, J = | (APCI⁺) |
| | ({[(2R)- | 1.7 Hz, 1H), 7.38-7.28 (m, 2H), 7.27-7.20 (m, | m/z 607 |
| | tetrahydrofuran-2- | 2H), 7.19-7.10 (m, 2H), 5.36-5.01 (m, 1H), 4.83 | (M + H)⁺ |
| | ylmethyl]carbamoyl}amino)- | (d, J = 17.6 Hz, 1H), 4.74-4.49 (m, 2H), 4.47- | |
| | 2,3-dihydro-3′H- | 4.27 (m, 1H), 3.88 (qd, J = 6.6, 4.5 Hz, 1H), 3.79 | |
| | spiro[indene-1,5′- | (dt, J = 8.2, 6.5 Hz, 1H), 3.69-3.60 (m, 1H), 3.22 | |
| | [1,3]oxazolidin]-3′-yl]- | (d, J = 4.6 Hz, 1H), 3.18-3.08 (m, 2H), 3.01 (ddd, | |
| | N-(4-fluorobenzyl)-N- | J = 16.4, 8.7, 4.1 Hz, 1H), 2.67 (ddd, J = 15.0, 8.7, | |
| | [(2S)-1,1,1- | 6.6 Hz, 1H), 2.50-2.43 (m, 1H), 1.97-1.76 (m, | |
| | trifluoropropan-2- | 3H), 1.55 (ddd, J = 11.0, 8.2, 5.5 Hz, 1H), 1.37 (d, | |
| | yl]acetamide | J = 7.0 Hz, 3H) | |
| 332 | 2-[(1R)-2′,4′-dioxo-5- | ¹H NMR (400 MHz, DMSO-d₆) δ ppm 7.53-7.44 | (APCI⁺) |
| | ({[2-(propan-2- | (m, 1H), 7.37-7.29 (m, 2H), 7.27-7.21 (m, 2H), | m/z 609 |
| | yloxy)ethyl]carbamoyl} | 7.15 (t, J = 7.7 Hz, 3H), 5.37-4.98 (m, 1H), 4.83 | (M + H)⁺ |
| | amino)-2,3-dihydro- | (d, J = 17.6 Hz, 1H), 4.73-4.51 (m, 2H), 4.45- | |
| | 3′H-spiro[indene-1,5′- | 4.27 (m, 1H), 3.59 (dt, J = 12.2, 6.1 Hz, 1H), 3.44 | |
| | [1,3]oxazolidin]-3′-yl]- | (t, J = 5.7 Hz, 2H), 3.23 (d, J = 5.7 Hz, 2H), 3.13 | |
| | N-(4-fluorobenzyl)-N- | (dt, J = 15.5, 7.6 Hz, 1H), 3.01 (ddd, J = 16.5, 8.7, | |
| | [(2S)-1,1,1- | 4.1 Hz, 1H), 2.67 (ddd, J = 15.1, 8.8, 6.6 Hz, 1H), | |
| | trifluoropropan-2- | 2.50-2.44 (m, 1H), 1.37 (d, J = 7.1 Hz, 3H), 1.11 | |
| | yl]acetamide | (d, J = 6.1 Hz, 6H) | |
| 333 | 2-{(1R)-5- | ¹H NMR (400 MHz, DMSO-d₆) δ ppm 7.54-7.45 | (APCI⁺) |
| | [(cyclopropylcarbamoyl)amino]- | (m, 1H), 7.38-7.29 (m, 2H), 7.27-7.22 (m, 2H), | m/z 563 |
| | 2′,4′-dioxo- | 7.15 (t, J = 8.7 Hz, 2H), 5.34-5.00 (m, 1H), 4.83 | (M + H)⁺ |
| | 2,3-dihydro-3′H- | (d, J = 17.8 Hz, 1H), 4.73-4.52 (m, 2H), 4.45- | |
| | spiro[indene-1,5′- | 4.29 (m, 1H), 3.18-3.09 (m, 1H), 3.01 (ddd, J = | |
| | [1,3]oxazolidin]-3′-yl}- | 16.5, 8.7, 4.1 Hz, 1H), 2.67 (ddd, J = 15.0, 8.7, 6.5 | |
| | N-(4-fluorobenzyl)-N- | Hz, 1H), 2.57 (tt, J = 7.1, 3.7 Hz, 1H), 2.50-2.45 | |
| | [(2S)-1,1,1- | (m, 1H), 1.37 (d, J = 7.0 Hz, 3H), 0.65 (td, J = 6.9, | |
| | trifluoropropan-2- | 4.7 Hz, 2H), 0.46-0.39 (m, 2H) | |
| | yl]acetamide | | |
| 334 | 2-[(1R)-5- | ¹H NMR (400 MHz, DMSO-d₆) δ ppm 7.53 (s, | (APCI⁺) |
| | {[ethyl(methyl)carbam- | 1H), 7.39-7.27 (m, 3H), 7.24 (dd, J = 8.3, 2.8 Hz, | m/z 565 |
| | oyl]amino}-2′,4′-dioxo- | 1H), 7.15 (t, J = 8.7 Hz, 2H), 5.37-5.02 (m, 1H), | (M + H)⁺ |
| | 2,3-dihydro-3′H- | 4.83 (d, J = 17.4 Hz, 1H), 4.74-4.50 (m, 2H), | |
| | spiro[indene-1,5′- | 4.45-4.26 (m, 1H), 3.35 (q, J = 7.1 Hz, 2H), 3.13 | |
| | [1,3]oxazolidin]-3′-yl]- | (dt, J = 15.5, 7.5 Hz, 1H), 3.01 (ddd, J = 16.2, 8.6, | |
| | N-(4-fluorobenzyl)-N- | 4.1 Hz, 1H), 2.92 (s, 3H), 2.67 (ddd, J = 15.0, 8.5, | |
| | [(2S)-1,1,1- | 6.5 Hz, 1H), 2.50-2.44 (m, 1H), 1.37 (d, J = 7.0 | |
| | trifluoropropan-2- | Hz, 3H), 1.09 (t, J = 7.1 Hz, 3H) | |
| | yl]acetamide | | |
| 335 | 2-{(1R)-5- | ¹H NMR (400 MHz, DMSO-d₆) δ ppm 7.54 (s, | (APCI⁺) |
| | [(diethylcarbamoyl)amino]- | 1H), 7.39-7.30 (m, 3H), 7.24 (dd, J = 8.4, 2.6 Hz, | m/z 579 |
| | 2′,4′-dioxo-2,3- | 1H), 7.15 (t, J = 8.0 Hz, 2H), 5.37-4.99 (m, 1H), | (M + H)⁺ |
| | dihydro-3′H- | 4.83 (d, J = 17.8 Hz, 1H), 4.73-4.50 (m, 2H), | |
| | spiro[indene-1,5′- | 4.46-4.28 (m, 1H), 3.35 (q, J = 7.0 Hz, 4H), 3.14 | |
| | [1,3]oxazolidin]-3′-yl}- | (dt, J = 15.3, 7.4 Hz, 1H), 3.01 (ddd, J = 16.4, 8.7, | |
| | N-(4-fluorobenzyl)-N- | 4.2 Hz, 1H), 2.67 (ddd, J = 14.9, 8.7, 6.6 Hz, 1H), | |
| | [(2S)-1,1,1- | 2.50-2.45 (m, 1H), 1.38 (d, J = 7.0 Hz, 3H), 1.11 | |
| | trifluoropropan-2- | (t, J = 7.0 Hz, 6H) | |
| | yl]acetamide | | |
| 336 | N-[(1R)-3′-(2-{(4- | ¹H NMR (400 MHz, DMSO-d₆) δ ppm 7.56 (d, J = | (APCI⁺) |
| | fluorobenzyl)[(2S)- | 1.8 Hz, 1H), 7.39 (dt, J = 8.7, 2.0 Hz, 1H), 7.36- | m/z 577 |
| | 1,1,1-trifluoropropan-2- | 7.29 (m, 2H), 7.24 (dd, J = 8.3, 3.0 Hz, 1H), 7.15 | (M + H)⁺ |
| | yl]amino}-2-oxoethyl)- | (t, J = 8.7 Hz, 2H), 5.37-5.06 (m, 1H), 4.83 (d, J = | |
| | 2′,4′-dioxo-2,3- | 17.5 Hz, 1H), 4.74-4.48 (m, 2H), 4.45-4.28 | |
| | dihydrospiro[indene- | (m, 1H), 3.42-3.36 (m, 4H), 3.13 (dt, J = 15.5, | |
| | 1,5′-[1,3]oxazolidin]-5- | 7.5 Hz, 1H), 3.01 (ddd, J = 16.3, 8.7, 4.1 Hz, 1H), | |
| | yl]pyrrolidine-1- | 2.67 (ddd, J = 15.0, 8.6, 6.5 Hz, 1H), 2.50-2.45 | |
| | carboxamide | (m, 1H), 1.91-1.83 (m, 4H), 1.37 (d, J = 7.0 Hz, | |
| | | 3H) | |
| 337 | 2-[(1R)-5-({[2- | ¹H NMR (400 MHz, DMSO-d₆) δ ppm 7.55 (d, J = | (APCI⁺) |
| | (dimethylamino)eth- | 1.5 Hz, 1H), 7.39 (dd, J = 8.4, 2.0 Hz, 1H), 7.36- | m/z 608 |
| | yl](methyl)carbamoyl}amino)- | 7.30 (m, 2H), 7.28 (d, J = 8.5 Hz, 1H), 7.15 (t, J = | (M + H)⁺ |
| | 2′,4′-dioxo-2,3- | 8.6 Hz, 2H), 5.32-5.02 (m, 1H), 4.83 (d, J = 17.6 | |
| | dihydro-3′H- | Hz, 1H), 4.73-4.49 (m, 2H), 4.46-4.26 (m, 1H), | |
| | spiro[indene-1,5′- | 3.65 (t, J = 6.2 Hz, 2H), 3.29 (d, J = 6.2 Hz, 2H), | |
| | [1,3]oxazolidin]-3′-yl]- | 3.15 (dt, J = 15.3, 7.5 Hz, 1H), 3.07-2.99 (m, | |
| | N-(4-fluorobenzyl)-N- | 4H), 2.87 (s, 6H), 2.74-2.63 (m, 2H), 1.38 (d, J = | |
| | [(2S)-1,1,1- | 7.0 Hz, 3H) | |
| | trifluoropropan-2- | | |
| | yl]acetamide | | |
| 338 | N²-{[(1R)-3′-(2-{(4- | ¹H NMR (400 MHz, DMSO-d₆) δ ppm 7.49 (d, J = | (APCI⁺) |
| | fluorobenzyl)[(2S)- | 1.7 Hz, 1H), 7.37-7.30 (m, 2H), 7.26 (d, J = 8.4 | m/z 636 |
| | 1,1,1-trifluoropropan-2- | Hz, 1H), 7.22 (dd, J = 8.4, 1.9 Hz, 1H), 7.15 (t, J = | (M + H)⁺ |
| | yl]amino}-2-oxoethyl)- | 8.7 Hz, 2H), 5.34-5.08 (m, 1H), 4.83 (d, J = 17.6 | |
| | 2′,4′-dioxo-2,3- | Hz, 1H), 4.73-4.50 (m, 2H), 4.44-4.29 (m, 1H), | |
| | dihydrospiro[indene- | 4.19 (dd, J = 9.0, 5.2 Hz, 1H), 3.13 (dt, J = 15.4, | |
| | 1,5′-[1,3]oxazolidin]-5- | 7.5 Hz, 1H), 3.01 (ddd, J = 16.5, 8.7, 4.1 Hz, 1H), | |
| | yl]carbamoyl}-L- | 2.67 (ddd, J = 15.0, 8.7, 6.6 Hz, 1H), 2.50-2.43 | |
| | leucinamide | (m, 1H), 1.74-1.62 (m, 1H), 1.56 (ddd, J = 13.3, | |
| | | 7.9, 5.2 Hz, 1H), 1.45 (ddd, J = 13.7, 9.0, 5.7 Hz, | |
| | | 1H), 1.37 (d, J = 7.0 Hz, 3H), 0.92 (dd, J = 6.6, 4.9 | |
| | | Hz, 6H) | |
| 339 | N-(4-fluorobenzyl)-2- | ¹H NMR (400 MHz, DMSO-d₆) δ ppm 7.46 (d, J = | (APCI⁺) |
| | [(1R)-5-{[(2- | 1.6 Hz, 1H), 7.38-7.30 (m, 2H), 7.28 (dd, J = 8.5, | m/z 609 |
| | hydroxyethyl)(propyl)car- | 2.0 Hz, 1H), 7.26-7.22 (m, 1H), 7.15 (t, J = 8.6 | (M + H)⁺ |
| | bamoyl]amino}-2′,4′- | Hz, 2H), 5.33-4.97 (m, 1H), 4.87-4.78 (m, 1H), | |
| | dioxo-2,3-dihydro-3′H- | 4.73-4.52 (m, 2H), 4.45-4.28 (m, 1H), 3.62 (t, J = | |
| | spiro[indene-1,5′- | 5.4 Hz, 2H), 3.41 (t, J = 5.5 Hz, 2H), 3.32-3.27 | |
| | [1,3]oxazolidin]-3′-yl]- | (m, 2H), 3.13 (dt, J = 15.4, 7.5 Hz, 1H), 3.01 (ddd, | |
| | N-[(2S)-1,1,1- | J = 16.4, 8.7, 4.2 Hz, 1H), 2.67 (ddd, J = 15.0, 8.7, | |
| | trifluoropropan-2- | 6.6 Hz, 1H), 2.50-2.44 (m, 1H), 1.55 (h, J = 7.4 | |
| | yl]acetamide | Hz, 2H), 1.37 (d, J = 7.0 Hz, 3H), 0.87 (t, J = 7.4 | |
| | | Hz, 3H) | |
| 340 | N-(4-fluorobenzyl)-2- | ¹H NMR (400 MHz, DMSO-d₆) δ ppm 7.48 (d, J = | (APCI⁺) |
| | [(1R)-5-{[(2- | 1.5 Hz, 1H), 7.38-7.30 (m, 2H), 7.27-7.21 (m, | m/z 581 |
| | methoxyethyl)carbam- | 2H), 7.15 (t, J = 8.7 Hz, 2H), 5.32-4.99 (m, 1H), | (M + H)⁺ |
| | oyl]amino}-2′,4′-dioxo- | 4.83 (d, J = 17.7 Hz, 1H), 4.74-4.51 (m, 2H), | |
| | 2,3-dihydro-3′H- | 4.46-4.28 (m, 1H), 3.42 (t, J = 5.6 Hz, 2H), 3.29 | |
| | spiro[indene-1,5′- | (s, 3H), 3.13 (dt, J = 15.4, 7.5 Hz, 1H), 3.01 (ddd, J = | |
| | [1,3]oxazolidin]-3′-yl]- | 16.5, 8.8, 4.1 Hz, 1H), 2.67 (ddd, J = 15.0, 8.7, | |
| | N-[(2S)-1,1,1- | 6.6 Hz, 1H), 2.50-2.44 (m, 1H), 1.37 (d, J = 7.0 | |
| | trifluoropropan-2- | Hz, 3H) | |
| | yl]acetamide | | |
| 341 | N-(4-fluorobenzyl)-2- | ¹H NMR (400 MHz, DMSO-d₆) δ ppm 7.53-7.44 | (APCI⁺) |
| | [(1R)-5-{[(3- | (m, 1H), 7.39-7.29 (m, 2H), 7.27-7.21 (m, 2H), | m/z 581 |
| | hydroxypropyl)carbam- | 7.15 (t, J = 8.7 Hz, 2H), 5.39-5.01 (m, 1H), 4.83 | (M + H)⁺ |
| | oyl]amino}-2′,4′-dioxo- | (d, J = 17.6 Hz, 1H), 4.74-4.47 (m, 2H), 4.46- | |
| | 2,3-dihydro-3′H- | 4.23 (m, 1H), 3.49 (t, J = 6.3 Hz, 2H), 3.17 (t, J = | |
| | spiro[indene-1,5′- | 6.9 Hz, 2H), 3.14-3.08 (m, 1H), 3.00 (ddd, J = | |
| | [1,3]oxazolidin]-3′-yl]- | 16.5, 8.7, 4.1 Hz, 1H), 2.67 (ddd, J = 15.0, 8.7, 6.6 | |
| | N-[(2S)-1,1,1- | Hz, 1H), 2.50-2.44 (m, 1H), 1.63 (p, J = 6.6 Hz, | |
| | trifluoropropan-2- | 2H), 1.37 (d, J = 6.9 Hz, 3H) | |
| | yl]acetamide | | |
| 342 | N-(4-fluorobenzyl)-2- | ¹H NMR (400 MHz, DMSO-d₆) δ ppm 7.50 (d, J = | (APCI⁺) |
| | [(1R)-5-({[(2S)-1- | 1.7 Hz, 1H), 7.39-7.29 (m, 2H), 7.25 (d, J = 8.4 | m/z 609 |
| | hydroxy-3- | Hz, 1H), 7.21 (dd, J = 8.4, 1.8 Hz, 1H), 7.15 (t, J = | (M + H)⁺ |
| | methylbutan-2- | 8.8 Hz, 2H), 5.38-4.94 (m, 1H), 4.83 (d, J = 17.6 | |
| | yl]carbamoyl}amino)- | Hz, 1H), 4.76-4.49 (m, 2H), 4.45-4.25 (m, 1H), | |
| | 2′,4′-dioxo-2,3-dihydro- | 3.47 (tt, J = 11.0, 5.2 Hz, 3H), 3.13 (dt, J = 15.4, | |
| | 3′H-spiro[indene-1,5′- | 7.4 Hz, 1H), 3.01 (ddd, J = 16.5, 8.7, 4.1 Hz, 1H), | |
| | [1,3]oxazolidin]-3′-yl]- | 2.67 (ddd, J = 15.0, 8.6, 6.6 Hz, 1H), 2.50-2.43 | |
| | N-[(2S)-1,1,1- | (m, 1H), 1.87 (h, J = 6.7 Hz, 1H), 1.37 (d, J = 7.0 | |
| | trifluoropropan-2- | Hz, 3H), 0.90 (dd, J = 11.0, 6.8 Hz, 6H) | |
| | yl]acetamide | | |
| 343 | N-(4-fluorobenzyl)-2- | ¹H NMR (400 MHz, DMSO-d₆) δ ppm 7.53 (d, J = | (APCI⁺) |
| | [(1R)-5- | 1.7 Hz, 1H), 7.39-7.28 (m, 3H), 7.24 (dd, J = 8.4, | m/z 579 |
| | {[methyl(propan-2- | 2.8 Hz, 1H), 7.15 (t, J = 8.9 Hz, 2H), 5.36-4.97 | (M + H)⁺ |
| | yl)carbamoyl]amino}- | (m, 1H), 4.83 (d, J = 17.5 Hz, 1H), 4.73-4.49 (m, | |
| | 2′,4′-dioxo-2,3-dihydro- | 2H), 4.47-4.28 (m, 2H), 3.13 (dt, J = 15.5, 7.4 | |
| | 3′H-spiro[indene-1,5′- | Hz, 1H), 3.01 (ddd, J = 16.3, 8.6, 4.2 Hz, 1H), 2.79 | |
| | [1,3]oxazolidin]-3′-yl]- | (s, 3H), 2.67 (ddd, J = 15.0, 8.7, 6.6 Hz, 1H), 1.37 | |
| | N-[(2S)-1,1,1- | (d, J = 7.0 Hz, 3H), 1.10 (d, J = 6.7 Hz, 6H) | |
| | trifluoropropan-2- | | |
| | yl]acetamide | | |
| 344 | 2-[(1R)-5-{[(2- | ¹H NMR (400 MHz, DMSO-d₆) δ 8.67 (d, J = 3.2 | (ESI(+)) |
| | cyanoethyl)carbamoyl] | Hz, 1H), 7.38 (d, J = 6.2 Hz, 1H), 7.22-6.88 (m, | m/e 593 |
| | amino}-2′,4′-dioxo-2,3- | 6H), 6.33 (t, J = 6.0 Hz, 1H), 5.04 (dp, J = 135.9, | (M + H)⁺ |
| | dihydro-3′H- | 7.6 Hz, 1H), 4.84-3.88 (m, 4H), 3.17 (d, J = 6.2 | |
| | spiro[indene-1,5′- | Hz, 2H), 3.01-2.64 (m, 2H), 2.50-2.42 (m, 2H), | |
| | [1,3]oxazolidin]-3′-yl]- | 2.38-2.16 (m, 2H), 1.17 (dd, J = 17.6, 6.9 Hz, | |
| | N-(4-fluorobenzyl)-N- | 3H) | |
| | [(2S)-1,1,1- | | |
| | trifluoropropan-2- | | |
| | yl]acetamide | | |
| 345 | N-(4-fluorobenzyl)-2- | ¹H NMR (400 MHz, DMSO-d₆) δ ppm 7.57-7.48 | (APCI⁺) |
| | [(1R)-5- | (m, 1H), 7.38-7.29 (m, 3H), 7.24 (dd, J = 8.4, 2.8 | m/z 579 |
| | {[methyl(propyl)carbam- | Hz, 1H), 7.15 (t, J = 8.8 Hz, 2H), 5.36-4.95 (m, | (M + H)⁺ |
| | oyl]amino}-2′,4′- | 1H), 4.83 (d, J = 17.6 Hz, 1H), 4.74-4.51 (m, | |
| | dioxo-2,3-dihydro-3′H- | 2H), 4.47-4.26 (m, 1H), 3.29 (d, J = 7.1 Hz, 2H), | |
| | spiro[indene-1,5′- | 3.13 (dt, J = 15.5, 7.4 Hz, 1H), 3.01 (ddd, J = 16.4, | |
| | [1,3]oxazolidin]-3′-yl]- | 8.7, 4.1 Hz, 1H), 2.93 (s, 3H), 2.67 (ddd, J = 15.0, | |
| | N-[(2S)-1,1,1- | 8.6, 6.5 Hz, 1H), 2.50-2.44 (m, 1H), 1.54 (h, J = | |
| | trifluoropropan-2- | 7.4 Hz, 2H), 1.37 (d, J = 7.0 Hz, 3H), 0.87 (t, J = | |
| | yl]acetamide | 7.4 Hz, 3H) | |
| 422 | N-(4-fluorobenzyl)-2- | ¹H NMR (400 MHz, DMSO-d₆) δ 8.83 (d, J = 3.8 | (ESI(+)) |
| | [(1R)-5-{[(1-methyl- | Hz, 1H), 8.46 (s, 1H), 7.73 (s, 1H), 7.64-7.53 (m, | m/e 625 |
| | 1H-pyrazol-4- | 1H), 7.46-7.19 (m, 6.3 H), 7.14 (t, J = 8.8 Hz, | (M + Na)⁺ |
| | yl)carbamoyl]amino}- | 0.7H), 5.40 (dd, J = 15.3, 7.5 Hz, 0.7H), 5.12-5.02 | |
| | 2′,4′-dioxo-2,3-dihydro- | (m, 0.3H), 5.03-4.70 (m, 2H), 4.63-4.40 (m, | |
| | 3′H-spiro[indene-1,5′- | 1.3H), 4.21 (d, J = 17.1 Hz, 0.6H), 3.19-2.95 (m, | |
| | [1,3]oxazolidin]-3′-yl]- | 2H), 2.69-2.51 (m, 2H), 1.36 (dd, J = 17.5, 6.9 | |
| | N-[(2S)-1,1,1- | Hz, 3H) | |
| | trifluoropropan-2- | | |
| | yl]acetamide | | |
| 426 | 2-{(1R)-2′,4′-dioxo-5- | ¹H NMR (400 MHz, DMSO-d₆) δ 8.84 (s, 1H), | (ESI(+)) |
| | [(phenylcarbamoyl)amino]- | 8.70 (s, 1H), 7.61 (d, J = 7.3 Hz, 1H), 7.28 (dddd, J = | m/e 621 |
| | 2,3-dihydro-3′H- | 58.7, 43.9, 20.0, 8.4 Hz, 9H), 6.96 (t, J = 7.3 Hz, | (M + Na)⁺ |
| | spiro[indene-1,5′- | 1H), 5.50-4.11 (m, 6H), 3.20-2.91 (m, 2H), 2.71- | |
| | [1,3]oxazolidin]-3′-yl}- | 2.56 (m, 1H), 1.35 (dd, J = 17.2, 6.9 Hz, 3H) | |
| | N-(4-fluorobenzyl)-N- | | |
| | [(2S)-1,1,1- | | |
| | trifluoropropan-2- | | |
| | yl]acetamide | | |
| 427 | 2-{(1R)-2′,4′-dioxo-5- | ¹H NMR (400 MHz, DMSO-d₆) δ 8.98 (d, J = 11.0 | (ESI(+)) |
| | [(pyridin-3- | Hz, 1H), 8.89 (s, 1H), 8.59 (d, J = 2.4 Hz, 1H), | m/e 622 |
| | ylcarbamoyl)amino]- | 8.18 (d, J = 4.4 Hz, 1H), 7.92 (dd, J = 5.0, 3.4 Hz, | (M + Na)⁺ |
| | 2,3-dihydro-3′H- | 1H), 7.61 (d, J = 6.7 Hz, 1H), 7.41-7.08 (m, 7H), | |
| | spiro[indene-1,5′- | 5.47-4.16 (m, 6H), 3.20-2.95 (m, 2H), 2.72- | |
| | [1,3]oxazolidin]-3′-yl}- | 2.57 (m, 1H) | |
| | N-(4-fluorobenzyl)-N- | | |
| | [(2S)-1,1,1- | | |
| | trifluoropropan-2- | | |
| | yl]acetamide | | |
| 548 | N-[3-({[(1R)-3′-(2-{(4- | ¹H NMR (400 MHz, DMSO-d₆) δ 8.86 (d, J = 4.0 | (ESI(+)) |
| | fluorobenzyl)[(2S)- | Hz, 1H), 7.84-7.02 (m, 8H), 6.32 (t, J = 6.3 Hz, | m/e 664 |
| | 1,1,1-trifluoropropan-2- | 1H), 5.54-4.12 (m, 5H), 3.18-2.81 (m, 6H), 2.70- | (M + H)⁺ |
| | yl]amino}-2-oxoethyl)- | 2.39 (m, 2H), 2.14 (qd, J = 7.6, 1.6 Hz, 2H), 1.36 | |
| | 2′,4′-dioxo-2,3- | (dd, J = 17.2, 6.8 Hz, 3H), 1.07-0.98 (m, 3H), | |
| | dihydrospiro[indene- | 0.79 (d, J = 2.0 Hz, 6H) | |
| | 1,5′-[1,3]oxazolidin]-5- | | |
| | yl]carbamoyl}amino)- | | |
| | 2,2-dimethylpropyl]propan- | | |
| | amide | | |
| 575 | 2-[(1R)-2′,4′-dioxo-5- | ¹H NMR (400 MHz, DMSO-d₆) δ 8.86 (d, J = 4.1 | (ESI(+)) |
| | ({[3-(pyrrolidin-1- | Hz, 1H), 8.75 (s, 1H), 7.61 (d, J = 7.0 Hz, 1H), | m/e 682 |
| | ylmethyl)phenyl]carbam- | 7.44 (s, 1H), 7.41-7.08 (m, 8H), 6.90 (d, J = 7.4 | (M + H)⁺ |
| | oyl}amino)-2,3- | Hz, 1H), 5.45-5.34 (m, 0.7H), 5.10-5.01 (m, | |
| | dihydro-3′H- | 0.3H), 5.00-4.70 (m, 2H), 4.60-4.40 (m, 1.3H), | |
| | spiro[indene-1,5′- | 4.20 (d, J = 17.1 Hz, 0.7H), 3.56 (s, 2H), 3.16- | |
| | [1,3]oxazolidin]-3′-yl]- | 2.94 (m, 2H), 2.62 (m, 2H), 2.46 (s, 4H), 1.69 (s, | |
| | N-(4-fluorobenzyl)-N- | 3H), 1.35 (dd, J = 17.3, 6.9 Hz, 3H) | |
| | [(2S)-1,1,1- | | |
| | trifluoropropan-2- | | |
| | yl]acetamide | | |
| 576 | N-(4-fluorobenzyl)-2- | ¹H NMR (400 MHz, DMSO-d₆) δ 8.80 (s, 1H), | (ESI(+)) |
| | [(1R)-5-({[3- | 8.71 (s, 1H), 7.59 (d, J = 7.1 Hz, 1H), 7.43-7.07 | m/e 698 |
| | (morpholin-4- | (m, 9H), 6.89 (t, J = 8.4 Hz, 1H), 5.37 (dd, J = | (M + H)⁺ |
| | ylmethyl)phenyl]carbam- | 15.5, 7.8 Hz, 0.7 H), 5.08-5.01 (m, 0.3H), 4.98- | |
| | oyl}amino)-2′,4′- | 4.65 (m, 2H), 4.58-4.37 (m, 1.3H), 4.18 (d, J = | |
| | dioxo-2,3-dihydro-3′H- | 17.1 Hz, 0.7H), 3.53 (dd, J = 10.3, 6.1 Hz, 4H), | |
| | spiro[indene-1,5′- | 3.39 (s, 2H), 3.16-2.93 (m, 2H), 2.68-2.50 (m, | |
| | [1,3]oxazolidin]-3′-yl]- | 2H), 2.35-2.25 (m, 4H), 1.33 (dd, J = 17.2, 6.9 | |
| | N-[(2S)-1,1,1- | Hz, 3H) | |
| | trifluoropropan-2- | | |
| | yl]acetamide | | |
| 577 | N³-{[(1R)-3′-(2-{(4- | ¹H NMR (501 MHz, DMSO-d₆) δ 8.78 (d, J = 4.8 | (ESI(+)) |
| | fluorobenzyl)[(2S)- | Hz, 1H), 7.83 (d, J = 5.0 Hz, 1H), 7.62-7.06 (m, | m/e 608 |
| | 1,1,1-trifluoropropan-2- | 7H), 6.38-6.12 (m, 1H), 5.50-4.13 (m, 5H), 3.33- | (M + H)⁺ |
| | yl]amino}-2-oxoethyl)- | 2.89 (m, 4H), 2.69-2.40 (m, 5H), 2.26 (td, J = | |
| | 2′,4′-dioxo-2,3- | 6.4, 2.7 Hz, 2H), 1.36 (dd, J = 21.7, 7.0 Hz, 3H) | |
| | dihydrospiro[indene- | | |
| | 1,5′-[1,3]oxazolidin]-5- | | |
| | yl]carbamoyl}-N- | | |
| | methyl-beta-alaninamide | | |
| 578 | N-[3-({[(1R)-3′-(2-{(4- | ¹H NMR (400 MHz, DMSO-d₆) δ 9.82 (s, 1H), | (ESI(+)) |
| | fluorobenzyl)[(2S)- | 8.79 (dd, J = 18.3, 3.3 Hz, 2H), 7.79 (d, J = 2.8 Hz, | m/e 670 |
| | 1,1,1-trifluoropropan-2- | 1H), 7.68-7.00 (m, 1H), 5.53-4.13 (m, 5H), 3.26- | (M + H)⁺ |
| | yl]amino}-2-oxoethyl)- | 2.94 (m, 2H), 2.75-2.41 (m, 2H), 2.31 (q, J = | |
| | 2′,4′-dioxo-2,3- | 7.5 Hz, 2H), 1.37 (dd, J = 16.8, 6.9 Hz, 3H), 1.08 | |
| | dihydrospiro[indene- | (t, J = 7.5 Hz, 3H) | |
| | 1,5′-[1,3]oxazolidin]-5- | | |
| | yl]carbamoyl}amino)phe- | | |
| | nyl]propanamide | | |
| 589 | 2-[(1R)-5-({[3- | ¹H NMR (501 MHz, DMSO-d₆) δ 8.75 (d, J = 5.8 | (ESI(+)) |
| | (acetylamino)propyl]car- | Hz, 1H), 7.83 (t, J = 5.5 Hz, 1H), 7.63-6.98 (m, | m/e 622 |
| | bamoyl}amino)-2′,4′- | 7H), 6.28 (q, J = 3.8, 2.1 Hz, 1H), 5.49-4.13 (m, | (M + H)⁺ |
| | dioxo-2,3-dihydro-3′H- | 5H), 3.19-2.87 (m, 6H), 2.70-2.40 (m, 2H), 1.80 | |
| | spiro[indene-1,5′- | (s, 3H), 1.53 (td, J = 6.9, 2.6 Hz, 2H), 1.36 (dd, J = | |
| | [1,3]oxazolidin]-3′-yl]- | 21.9, 6.9 Hz, 3H) | |
| | N-(4-fluorobenzyl)-N- | | |
| | [(2S)-1,1,1- | | |
| | trifluoropropan-2- | | |
| | yl]acetamide | | |
| 590 | 2-[(1R)-5-({[2- | ¹H NMR (501 MHz, DMSO-d₆) δ 8.75 (d, J = 4.3 | (ESI(+)) |
| | (acetylamino)ethyl]car- | Hz, 1H), 7.92 (t, J = 4.8 Hz, 1H), 7.65-7.09 (m, | m/e 608 |
| | bamoyl}amino)-2′,4′- | 7H), 6.23 (d, J = 5.4 Hz, 1H), 5.55-4.10 (m, 5H), | (M + H)⁺ |
| | dioxo-2,3-dihydro-3′H- | 3.19-2.91 (m, 6H), 2.74-2.35 (m, 2H), 1.81 (s, | |
| | spiro[indene-1,5′- | 3H), 1.36 (dd, J = 21.7, 6.9 Hz, 3H) | |
| | [1,3]oxazolidin]-3′-yl]- | | |
| | N-(4-fluorobenzyl)-N- | | |
| | [(2S)-1,1,1- | | |
| | trifluoropropan-2- | | |
| | yl]acetamide | | |
| 591 | tert-butyl [3-({[(1R)-3′- | ¹H NMR (501 MHz, DMSO-d₆) δ 8.80 (d, J = 5.5 | (ESI(+)) |
| | (2-{(4- | Hz, 1H), 7.67-7.50 (m, 1H), 7.48-7.08 (m, 6H), | m/e 708 |
| | fluorobenzyl)[(2S)- | 6.87-6.69 (m, 1H), 6.27 (t, J = 6.3 Hz, 1H), 5.49- | (M + H)⁺ |
| | 1,1,1-trifluoropropan-2- | 4.09 (m, 5H), 3.18-2.94 (m, 2H), 2.89 (dd, J = | |
| | yl]amino}-2-oxoethyl)- | 6.1, 3.1 Hz, 2H), 2.77 (dd, J = 6.5, 2.7 Hz, 2H), | |
| | 2′,4′-dioxo-2,3- | 2.71-2.43 (m, 2H), 1.39 (s, 9H), 1.38-1.31 (m, | |
| | dihydrospiro[indene- | 3H), 0.77 (s, 6H) | |
| | 1,5′-[1,3]oxazolidin]-5- | | |
| | yl]carbamoyl}amino)- | | |
| | 2,2-dimethylpropyl]carbamate | | |
| 592 | N-[2-({[(1R)-3′-(2-{(4- | ¹H NMR (400 MHz, DMSO-d₆) δ 8.75 (d, J = 3.4 | (ESI(+)) |
| | fluorobenzyl)[(2S)- | Hz, 1H), 7.82 (s, 1H), 7.62-7.03 (m, 7H), 6.24 (s, | m/e 622 |
| | 1,1,1-trifluoropropan-2- | 1H), 5.51-4.11 (m, 5H), 3.20-2.92 (m, 6H), 2.74- | (M + H)⁺ |
| | yl]amino}-2-oxoethyl)- | 2.40 (m, 2H), 2.15-1.99 (m, 2H), 1.36 (dd, J = | |
| | 2′,4′-dioxo-2,3- | 17.2, 7.0 Hz, 3H), 0.99 (td, J = 7.6, 1.7 Hz, 3H) | |
| | dihydrospiro[indene- | | |
| | 1,5′-[1,3]oxazolidin]-5- | | |
| | yl]carbamoyl}amino)eth- | | |
| | yl]propanamide | | |
| 594 | 2-[(1R)-5-({[3- | ¹H NMR (501 MHz, DMSO-d₆) δ 9.91 (s, 1H), | (ESI(+)) |
| | (acetylamino)phenyl]car- | 8.81 (dd, J = 13.1, 4.7 Hz, 2H), 7.83-7.06 (m, | m/e 656 |
| | bamoyl}amino)-2′,4′- | 11H), 5.49-4.11 (m, 5H), 3.24-2.89 (m, 2H), | (M + H)⁺ |
| | dioxo-2,3-dihydro-3′H- | 2.73-2.41 (m, 2H), 2.03(s, 3H), 1.37 (dd, J = | |
| | spiro[indene-1,5′- | 21.2, 6.9 Hz, 3H) | |
| | [1,3]oxazolidin]-3′-yl]- | | |
| | N-(4-fluorobenzyl)-N- | | |
| | [(2S)-1,1,1- | | |
| | trifluoropropan-2- | | |
| | yl]acetamide | | |
| 671 | 2-[(1R)-5- | ¹H NMR (400 MHz, DMSO-d₆) δ 9.10 (s, 1H), | (ESI(+)) |
| | {[(cyanomethyl)carbam- | 7.54 (d, J = 5.8 Hz, 1H), 7.38-7.05 (m, 6H), 6.75 | m/e 579 |
| | oyl]amino}-2′,4′-dioxo- | (t, J = 5.8 Hz, 1H), 5.37 (dt, J = 15.5, 7.7 Hz, | (M + NH₄)⁺ |
| | 2,3-dihydro-3′H- | 0.6H), 5.11-4.66 (m, 2.4H), 4.61-4.34 (m, 1.4H), | |
| | spiro[indene-1,5′- | 4.18 (d, J = 17.1 Hz, 0.6H), 4.10 (d, J = 5.8 Hz, | |
| | [1,3]oxazolidin]-3′-yl]- | 2H), 3.10 (dt, J = 15.0, 6.3 Hz, 1H), 2.97 (ddd, J = | |
| | N-(4-fluorobenzyl)-N- | 16.4, 8.4, 3.5 Hz, 1H), 2.67-2.39 (m, 2H), 1.33 | |
| | [(2S)-1,1,1- | (dd, J = 17.3, 6.9 Hz, 3H) | |
| | trifluoropropan-2- | | |
| | yl]acetamide | | |

### Example 346

### 2-[5′-(acetylamino)-2,5-dioxo-1′,3′-dihydro-1H-spiro[imidazolidine-4,2′-inden]-1-yl]-N-[(1S)-1-cyclopropylethyl]-N-(4-fluorobenzyl)acetamide

A solution of 2-(5′-amino-2,5-dioxo-1′,3′-dihydro-1H-spiro[imidazolidine-4,2′-inden]-1-yl)-N-[(1S)-1-cyclopropylethyl]-N-(4-fluorobenzyl)acetamide (0.034 g, 0.075 mmol) in dichloromethane (0.6 mL) cooled in an ice bath at 0° C. was treated with triethylamine (0.018 ml, 0.128 mmol) followed by acetyl chloride (7.70 mg, 0.098 mmol) and the reaction mixture was allowed to stir at room temperature for 15 hours. The reaction mixture was concentrated and the residue was purified by silica gel column chromatography to afford the title compound. ¹H NMR (400 MHz, DMSO-d₆, 90° C.) δ ppm 9.53 (s, 1H), 8.38 (s, 1H), 7.46 (d, J=1.9 Hz, 1H), 7.43-7.24 (m, 3H), 7.16-6.95 (m, 3H), 4.62 (brs, 2H), 4.34-4.10 (m, 2H), 3.64-3.40 (m, 1H), 3.34 (dd, J=16.6, 9.0 Hz, 2H), 3.08 (dd, J=16.6, 11.3 Hz, 2H), 2.01 (s, 3H), 1.18-1.11 (m, 3H), 1.00-0.88 (m, 1H), 0.56-0.41 (m, 1H), 0.30-0.14 (m, 3H). MS (ESI⁺) m/z 493 (M+H)⁺.

### Example 347

### N-[(1S)-1-cyclopropylethyl]-N-(4-fluorobenzyl)-2-{6-[(methylcarbamoyl)amino]-1,1-dioxido-2′,5′-dioxo-1′H-spiro[1-benzothiophene-3,4′-imidazolidin]-1′-yl}acetamide

The title compound was prepared as described in EXAMPLE 282, substituting 2-(6-amino-1,1-dioxido-2′,5′-dioxo-1′H-spiro[1-benzothiophene-3,4′-imidazolidin]-1′-yl)-N-[(1S)-1-cyclopropylethyl]-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆, 90° C.) δ ppm 8.93 (brs, 1H), 8.84 (s, 1H), 7.99 (d, J=1.9 Hz, 1H), 7.66-7.47 (m, 2H), 7.44-7.23 (m, 2H), 7.23-7.02 (m, 2H), 6.14-6.04 (m, 1H), 4.66 (brs, 2H), 4.49-4.15 (m, 2H), 3.92 (d, J=14.4 Hz, 1H), 3.83-3.29 (m, 2H), 2.68 (d, J=4.6 Hz, 3H), 1.22-1.12 (m, 3H), 1.04-0.89 (m, 1H), 0.53-0.43 (m, 1H), 0.36-0.14 (m, 3H). MS (ESI⁺) m/z 558 (M+H)⁺.

### Example 348

### N-(4-fluorobenzyl)-2-(1′-methyl-2,5-dioxo-2′,3′-dihydro-1H,1′H-spiro[imidazolidine-4,4′-quinolin]-1-yl)-N-[(2S)-1,1,1-trifluoropropan-2-yl]acetamide

### Example 348A

### 1-methyl-2,3-dihydroquinolin-4(1H)-one

A pressure tube was charged with 2,3-dihydroquinolin-4(1H)-one (570 mg, 3.87 mmol,), potassium carbonate (1.6 g, 11.58 mmol) and acetone (7.7 ml). Iodomethane (0.98 ml, 15.74 mmol) was added dropwise and the pressure tube was sealed and was heated at 80° C. for 16 hours. After cooling to room temperature, water and ethyl acetate were added. The layers were separated and the aqueous layer was extracted with ethyl acetate. The combined extracts were washed with brine, dried (magnesium sulfate), filtered and concentrated. Silica gel column chromatography gave the title compound.

### Example 348B

### 1′-methyl-2′,3′-dihydro-1′H-spiro[imidazolidine-4,4′-quinoline]-2,5-dione

A suspension of 1-methyl-2,3-dihydroquinolin-4(1H)-one (0.42 g, 2.61 mmol), potassium cyanide (0.339 g, 5.21 mmol), ammonium carbonate (1.25 g, 13.03 mmol), ethanol (6 ml) and water (6 ml) was heated at 100° C. in a 25 mL stainless steel reactor for 16 hours. The suspension was concentrated to dryness, water and ethyl acetate were added, and the mixture was extracted with ethyl acetate. The combined extracts were washed with water and brine, dried (magnesium sulfate), filtered and concentrated. The crude product contained starting material and was resubmitted to the above conditions twice more, processing each time as described above. The final crude product was purified using silica gel column chromatography to give the title compound.

### Example 348C

### N-(4-fluorobenzyl)-2-(1′-methyl-2,5-dioxo-2′,3′-dihydro-1H,1′H-spiro[imidazolidine-4,4′-quinolin]-1-yl)-N-[(2S)-1,1,1-trifluoropropan-2-yl]acetamide

A suspension of 1′-methyl-2′,3′-dihydro-1′H-spiro[imidazolidine-4,4′-quinoline]-2,5-dione (30 mg, 0.130 mmol), (S)-2-bromo-N-(4-fluorobenzyl)-N-(1,1,1-trifluoropropan-2-yl)acetamide (45 mg, 0.132 mmol), potassium carbonate (27 mg, 0.195 mmol) and N,N-dimethylformamide (0.90 ml) was stirred at room temperature for 16 hours. Water and ethyl acetate were added and the solution was extracted with ethyl acetate. The combined extracts were washed with water and brine, dried (magnesium sulfate), filtered and concentrated. The crude product was silica gel column chromatographed to provide the title compound. ¹H NMR (400 MHz, DMSO-d₆) δ 8.89 8.80 (m, 1H), 7.37 (s, 1H), 7.21 (t, J=8.8 Hz, 2H), 7.12 (td, J=10.2, 8.4, 4.2 Hz, 3H), 6.68 (d, J=8.4 Hz, 1H), 6.59 (q, J=7.7 Hz, 1H), 5.42-5.31 (m, 1H), 4.93-4.64 (m, 2H), 4.39 (dd, J=17.0, 5.3 Hz, 1H), 4.11-3.98 (m, 1H), 3.20 (d, J=8.1 Hz, 1H), 2.87 (d, J=2.9 Hz, 3H), 2.15 (t, J=11.0 Hz, 1H), 1.99 (d, J=14.1 Hz, 1H), 1.37-1.21 (m, 4H). MS ESI⁺ 493 (M+H)⁺.

### Example 349

### N-benzyl-N-[(1S)-1-cyclopropylethyl]-2-{7-[(methylcarbamoyl)amino]-2′,4′-dioxo-2,3-dihydro-3′H-spiro[chromene-4,5′-[1,3]oxazolidin]-3′-yl}acetamide

The title compound was prepared as described in EXAMPLE 282, substituting 2-(7-amino-2′,4′-dioxo-2,3-dihydro-3′H-spiro[chromene-4,5′-[1,3]oxazolidin]-3′-yl)-N-benzyl-N-[(1S)-1-cyclopropylethyl]acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆, 90° C.) δ ppm 8.38 (brs, 1H), 7.58-7.14 (m, 6H), 7.11 (d, J=2.0 Hz, 1H), 6.92-6.82 (m, 1H), 5.96-5.87 (m, 1H), 4.71 (brs, 2H), 4.62-4.19 (m, 3H), 4.12 (td, J=10.9, 3.3 Hz, 1H), 3.91-3.30 (m, 1H), 2.64 (d, J=4.6 Hz, 3H), 2.45-2.28 (m, 2H), 1.23-1.12 (m, 3H), 1.04-0.92 (m, 1H), 0.55-0.43 (m, 1H), 0.39-0.14 (m, 3H). MS (ESI⁺) m/z 529 (M+Na)⁺.

### Example 350

### 2-[7-(acetylamino)-2′,4′-dioxo-2,3-dihydro-3′H-spiro[chromene-4,5′-[1,3]oxazolidin]-3′-yl]-N-benzyl-N-[(1S)-1-cyclopropylethyl]acetamide

The title compound was prepared as described in EXAMPLE 346, substituting 2-(7-amino-2′,4′-dioxo-2,3-dihydro-3′H-spiro[chromene-4,5′-[1,3]oxazolidin]-3′-yl)-N-benzyl-N-[(1S)-1-cyclopropylethyl]acetamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (400 MHz, DMSO-d₆, 90° C.) δ ppm 9.74 (s, 1H), 7.53-7.11 (m, 7H), 7.06 (dd, J=8.6, 2.1 Hz, 1H), 4.71 (brs, 2H), 4.66-4.27 (m, 3H), 4.22-4.09 (m, 1H), 3.87-3.33 (m, 1H), 2.45-2.32 (m, 2H), 2.02 (s, 3H), 1.22-1.13 (m, 3H), 1.03-0.91 (m, 1H), 0.55-0.45 (m, 1H), 0.36-0.17 (m, 3H). MS (ESI⁺) m/z 514 (M+Na)⁺.

### Example 351

### N-[(1S)-1-cyclopropylethyl]-N-(4-fluorobenzyl)-2-(3′-hydroxy-2,5-dioxo-2′,3′-dihydro-1H-spiro[imidazolidine-4,1′-inden]-1-yl)acetamide

### Example 351A

### 3-hydroxy-2,3-dihydro-1H-inden-1-one

A mixture of 2-bromobenzaldehyde (5 g, 27.0 mmol), 1-(vinyloxy)butane (8.12 g, 81 mmol), palladium(II) acetate (0.121 g, 0.540 mmol), 1,3-bis(diphenylphosphino)propane (0.334 g, 0.811 mmol) in 100 ml ethylene glycol was degassed under vacuum and purged with nitrogen. Triethylamine (5.65 ml, 40.5 mmol) was added and the mixture was stirred at 115° C. overnight. The mixture was cooled to room temperature, and 100 mL aqueous 2M HCl was added. The mixture was stirred at room temperature for 1 hour and was extracted with ethyl acetate. The organic layer was washed with brine, dried over Na₂SO₄, filtered and concentrated. Silica gel column chromatography gave the title compound.

### Example 351B

### 3′-hydroxy-2′,3′-dihydrospiro[imidazolidine-4,1′-indene]-2,5-dione

The title compound was prepared as described in EXAMPLE 290B, substituting 3-hydroxy-2,3-dihydro-1H-inden-1-one for 6-bromobenzo[b]thiophen-3 (2H)-one 1,1-dioxide.

### Example 351C

### N-[(1S)-1-cyclopropylethyl]-N-(4-fluorobenzyl)-2-(3′-hydroxy-2,5-dioxo-2′,3′-dihydro-1H-spiro[imidazolidine-4,1′-inden]-1-yl)acetamide

The title compound was prepared as described in EXAMPLE 281F, substituting 3′-hydroxy-2′,3′-dihydrospiro[imidazolidine-4,1′-indene]-2,5-dione for (R)-5-bromo-2,3-dihydrospiro[indene-1,5′-oxazolidine]-2′,4′-dione and (S)-2-bromo-N-(1-cyclopropylethyl)-N-(4-fluorobenzyl)acetamide for (S)-2-bromo-N-(4-fluorobenzyl)-N-(1,1,1-trifluoropropan-2-yl)acetamide. ¹H NMR (400 MHz, DMSO-d₆) δ 8.78 (dd, J=27.3, 14.2 Hz, 1H), 7.55-6.98 (m, 8H), 5.79-5.03 (m, 2H), 4.89-4.01 (m, 4H), 2.85-2.72 (m, 1H), 2.40-1.87 (m, 1H), 1.29-1.04 (m, 3H), 1.01-0.04 (m, 5H). MS (ESI⁺) m/z 452 (M+NH4)⁺.

### Example 352

### N-[(1S)-1-cyclopropylethyl]-2-[(1R)-2′,4′-dioxo-5-(pyridin-4-yl)-2,3-dihydro-3′H-spiro[indene-1,5′-[1,3]oxazolidin]-3′-yl]-N-(4-fluorobenzyl)acetamide

The title compound was prepared as described in EXAMPLE 295, substituting 2-((R)-5-bromo-2′,4′-dioxo-2,3-dihydrospiro[indene-1,5′-oxazolidin]-3′-yl)-N—((S)-1-cyclopropylethyl)-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 4-(4,4,5,5-tetramethyl-1,3,2-dioxaborolan-2-yl)pyridine for 5-(4,4,5,5-tetramethyl-1,3,2-dioxaborolan-2-yl)pyridin-2-ol. ¹H NMR (400 MHz, DMSO-d₆, 90° C.) δ ppm 8.68-8.58 (m, 2H), 7.78 (d, J=1.6 Hz, 1H), 7.71 (dd, J=8.1, 1.7 Hz, 1H), 7.69-7.64 (m, 2H), 7.60 (d, J=8.1 Hz, 1H), 7.50-7.25 (m, 2H), 7.23-7.02 (m, 2H), 4.76-4.63 (m, 2H), 4.63-4.26 (m, 2H), 3.82-3.32 (m, 1H), 3.32-3.22 (m, 1H), 3.22-3.11 (m, 1H), 2.83-2.67 (m, 1H), 2.65-2.53 (m, 1H), 1.23-1.14 (m, 3H), 1.07-0.90 (m, 1H), 0.58-0.45 (m, 1H), 0.38-0.20 (m, 3H). MS (ESI⁺) m/z 514 (M+H)⁺.

### Example 353

### N-[(1S)-1-cyclopropylethyl]-N-(4-fluorobenzyl)-2-[(1R)-5-(6-hydroxypyridazin-3-yl)-2′,4′-dioxo-2,3-dihydro-3′H-spiro[indene-1,5′-[1,3]oxazolidin]-3′-yl]acetamide

### Example 353A

### N—((S)-1-cyclopropylethyl)-2-((R)-2′,4′-dioxo-5-(4,4,5,5-tetramethyl-1,3,2-dioxaborolan-2-yl)-2,3-dihydrospiro[indene-1,5′-oxazolidin]-3′-yl)-N-(4-fluorobenzyl)acetamide

A solution of 2-((R)-5-bromo-2′,4′-dioxo-2,3-dihydrospiro[indene-1,5′-oxazolidin]-3′-yl)-N—((S)-1-cyclopropylethyl)-N-(4-fluorobenzyl)acetamide (0.15 g, 0.291 mmol), 4,4,4′,4′,5,5,5′,5′-octamethyl-2,2′-bi(1,3,2-dioxaborolane) (0.185 g, 0.728 mmol), potassium acetate (0.086 g, 0.873 mmol) and 1,1′-bis(diphenylphosphino)ferrocene-palladium(II)dichloride dichloromethane complex (0.021 g, 0.026 mmol) in anhydrous dimethylsulfoxide (2 mL) was degassed by bubbling nitrogen through the mixture for 15 minutes. The reaction mixture was heated at 95° C. under a nitrogen atmosphere for 8 hours. The reaction was cooled to room temperature and partitioned between ethyl acetate and water. The aqueous layer was extracted with ethyl acetate and the combined organic layers were washed with brine, dried over anhydrous sodium sulfate, filtered, and concentrated. The concentrate was purified by silica gel column chromatography to provide the title compound.

### Example 353B

### N-[(1S)-1-cyclopropylethyl]-N-(4-fluorobenzyl)-2-[(1R)-5-(6-hydroxypyridazin-3-yl)-2′,4′-dioxo-2,3-dihydro-3′H-spiro[indene-1,5′-[1,3]oxazolidin]-3′-yl]acetamide

The title compound was prepared as described in EXAMPLE 295, substituting 6-chloropyridazin-3-ol for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N—((S)-1-cyclopropylethyl)-2-((R)-2′,4′-dioxo-5-(4,4,5,5-tetramethyl-1,3,2-dioxaborolan-2-yl)-2,3-dihydrospiro[indene-1,5′-oxazolidin]-3′-yl)-N-(4-fluorobenzyl)acetamide for 5-(4,4,5,5-tetramethyl-1,3,2-dioxaborolan-2-yl)pyridin-2-ol. ¹H NMR (400 MHz, DMSO-d₆, 90° C.) δ ppm 12.95 (brs, 1H), 7.94 (d, J=9.9 Hz, 1H), 7.85 (d, J=1.6 Hz, 1H), 7.78 (dd, J=7.9, 1.7 Hz, 1H), 7.55 (d, J=8.1 Hz, 1H), 7.45-7.24 (m, 2H), 7.22-7.04 (m, 2H), 6.94 (d, J=9.9 Hz, 1H), 4.69 (brs, 2H), 4.62-4.29 (m, 2H), 3.84-3.32 (m, 1H), 3.30-3.09 (m, 2H), 2.82-2.69 (m, 1H), 2.62-2.51 (m, 1H), 1.19 (d, J=6.6 Hz, 3H), 1.07-0.90 (m, 1H), 0.56-0.44 (m, 1H), 0.38-0.19 (m, 3H). MS (ESI⁺) m/z 531 (M+H)⁺.

### Example 354

### N-[(1S)-1-cyclopropylethyl]-2-[(1R)-2′,4′-dioxo-5-(1H-pyrazol-4-yl)-2,3-dihydro-3′H-spiro[indene-1,5′-[1,3]oxazolidin]-3′-yl]-N-(4-fluorobenzyl)acetamide

The title compound was prepared as described in EXAMPLE 295, substituting 2-((R)-5-bromo-2′,4′-dioxo-2,3-dihydrospiro[indene-1,5′-oxazolidin]-3′-yl)-N—((S)-1-cyclopropylethyl)-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆, 90° C.) δ ppm 12.74 (brs, 1H), 8.21-7.69 (m, 2H), 7.59 (s, 1H), 7.53 (dd, J=8.1, 1.6 Hz, 1H), 7.48-7.26 (m, 3H), 7.21-6.97 (m, 2H), 4.69 (brs, 2H), 4.62-4.17 (m, 2H), 3.84-3.31 (m, 1H), 3.28-3.02 (m, 2H), 2.77-2.63 (m, 1H), 2.58-2.49 (m, 1H), 1.19 (d, J=6.6 Hz, 3H), 1.04-0.91 (m, 1H), 0.54-0.45 (m, 1H), 0.45-0.16 (m, 3H). MS (ESI⁺) m/z 503 (M+H)⁺.

### Example 355

### N-(4-fluorobenzyl)-N-[(2S)-1,1,1-trifluoropropan-2-yl]-2-(2,3′,5-trioxo-2′,3′-dihydro-1H-spiro[imidazolidine-4,1′-inden]-1-yl)acetamide

### Example 355A

### spiro[imidazolidine-4,1′-indene]-2,3′,5(2′H)-trione

To 3′-hydroxy-2′,3′-dihydrospiro[imidazolidine-4,1′-indene]-2,5-dione (300 mg, 1.375 mmol) in 5 mL acetone was added Jone's reagent dropwise until the yellow color persisted. The mixture was stirred at room temperature for 30 minutes and was diluted with isopropanol. The resulting suspension was filtered and the filtrate was diluted with water and extracted with dichloromethane. The organic layer was dried with Na₂SO₄, filtered and concentrated. Silica gel column chromatography gave the title compound.

### Example 355B

### N-(4-fluorobenzyl)-N-[(2S)-1,1,1-trifluoropropan-2-yl]-2-(2,3′,5-trioxo-2′,3′-dihydro-1H-spiro[imidazolidine-4,1′-inden]-1-yl)acetamide

The title compound was prepared as described in EXAMPLE 281F, substituting spiro[imidazolidine-4, 1′-indene]-2,3′,5(2′H)-trione for (R)-5-bromo-2,3-dihydrospiro[indene-1,5′-oxazolidine]-2′,4′-dione. ¹H NMR (400 MHz, DMSO-d₆) δ 8.96 (dd, J=9.5, 3.2 Hz, 1H), 7.98-7.57 (m, 4H), 7.46-7.00 (m, 4H), 5.22 (ddt, J=122.4, 15.0, 7.6 Hz, 1H), 4.98-3.99 (m, 4H), 3.21-2.88 (m, 2H), 1.50-1.19 (m, 3H). MS (ESI⁺) m/z 478.0 (M+H)⁺.

### Example 356

### N-((1S)-1-cyclopropylethyl)-N-(4-fluorobenzyl)-2-(1′-methyl-2,5-dioxo-2′,3′-dihydro-1H,1′H-spiro[imidazolidine-4,4′-quinolin]-1-yl)acetamide

The title compound was prepared as described in EXAMPLE 348, substituting (S)-2-bromo-N-(1-cyclopropylethyl)-N-(4-fluorobenzyl)acetamide for (S)-2-bromo-N-(4-fluorobenzyl)-N-(1,1,1-trifluoropropan-2-yl)acetamide. ¹H NMR (400 MHz, DMSO-d₆) δ 8.82 (d, J=17.5 Hz, 1H), 7.44 (dd, J=8.5, 5.4 Hz, 1H), 7.34-7.03 (m, 5H), 6.75-6.54 (m, 2H), 4.73 (s, 1H), 4.60 (s, 1H), 4.50-4.07 (m, 2H), 3.72 (m, 1H), 3.22 (m, 1H), 2.89 (d, J=2.8 Hz, 3H), 2.18 (ddt, J=13.0, 9.3, 4.7 Hz, 1H), 2.02 (dtd, J=12.8, 9.6, 8.2, 4.5 Hz, 1H), 1.20 (d, J=6.2 Hz, 1H), 1.10 (d, J=6.9 Hz, 2H), 0.94 (ddt, J=12.7, 9.3, 4.1 Hz, 1H), 0.55-0.07 (m, 4H). MS ESI⁺ 465 (M+H)⁺.

### Example 357

### N-(4-fluorobenzyl)-2-[(3′E)-3′-(hydroxyimino)-2,5-dioxo-2′,3′-dihydro-1H-spiro[imidazolidine-4,1′-inden]-1-yl]-N-[(2S)-1,1,1-trifluoropropan-2-yl]acetamide

A mixture of N-(4-fluorobenzyl)-N-[(2S)-1,1,1-trifluoropropan-2-yl]-2-(2,3′,5-trioxo-2′,3′-dihydro-1H-spiro[imidazolidine-4,1′-inden]-1-yl)acetamide (50 mg, 0.105 mmol), hydroxylamine hydrochloride (8.73 mg, 0.126 mmol) and sodium acetate (17.18 mg, 0.209 mmol) in 2 mL methanol was stirred at room temperature overnight. The mixture was diluted with water and filtered with water washes to give the title compound after vacuum drying. ¹H NMR (400 MHz, DMSO-d₆) δ 11.49-11.03 (m, 1H), 9.14-8.63 (m, 1H), 7.78-6.97 (m, 8H), 5.37 (p, J=8.0 Hz, 1H), 4.98-3.86 (m, 4H), 3.28 (m, J=10.5 Hz, 1H), 3.04-2.78 (m, 1H), 1.52-1.15 (m, 3H). MS (ESI⁺) m/z 493.0 (M+H)⁺.

### Example 358

### N-[(1S)-1-cyclopropylethyl]-N-(4-fluorobenzyl)-2-(2, 3′,5-trioxo-2′,3′-dihydro-1H-spiro[imidazolidine-4,1′-inden]-1-yl)acetamide

N-[(1S)-1-Cyclopropylethyl]-2-[(4R)-2,5-dioxo-2′,3′-dihydro-H-spiro[imidazolidine-4,1′-inden]-1-yl]-N-(4-fluorobenzyl)acetamide (32 mg, 0.071 mmol) in 2 mL acetone was treated with Jone's reagent dropwise until the yellow color persisted. The mixture was stirred at room temperature for 30 minutes and then diluted with isopropanol. The resulting suspension was filtered and the filtrate was diluted with water and extracted with dichloromethane. The organic layer was dried with Na₂SO₄, filtered and concentrated. Silica gel column chromatography gave the title compound. ¹H NMR (400 MHz, DMSO-d6) δ 8.92 (d, J=13.9 Hz, 1H), 7.94-7.56 (m, 4H), 7.48-7.00 (m, 4H), 4.77-4.09 (m, 4H), 3.72-3.4 (m 1H), 3.10 (dd, J=18.6, 8.6 Hz, 1H), 2.93 (dd, J=18.7, 14.3 Hz, 1H), 1.28-1.04 (m, 3H), 1.06-0.09 (m, 5H). MS (ESI⁺) m/z 472.0 (M+Na)⁺.

### Example 359

### N-benzyl-N-[(1S)-1-cyclopropylethyl]-2-[2′,4′-dioxo-7-(6-oxo-1,6-dihydropyridin-3-yl)-2,3-dihydro-3′H-spiro[chromene-4,5′-[1,3]oxazolidin]-3′-yl]acetamide

The title compound was prepared as described in EXAMPLE 295, substituting N-benzyl-2-(7-bromo-2′,4′-dioxo-2,3-dihydro-3′H-spiro[chromene-4,5′-[1,3]oxazolidin]-3′-yl)-N-[(1S)-1-cyclopropylethyl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide. ¹H NMR (400 MHz, DMSO-d₆, 90° C.) δ ppm 7.76 (dd, J=9.5, 2.8 Hz, 1H), 7.69 (d, J=2.8 Hz, 1H), 7.44 (dd, J=8.2, 2.4 Hz, 1H), 7.42-7.17 (m, 5H), 7.15 (dd, J=8.2, 1.9 Hz, 1H), 7.07 (d, J=1.9 Hz, 1H), 6.39 (d, J=9.5 Hz, 1H), 4.72 (brs, 2H), 4.57-4.28 (m, 3H), 4.25-4.12 (m, 1H), 3.73 (s, 1H), 2.47-2.35 (m, 2H), 1.18 (d, J=6.7 Hz, 3H), 1.07-0.91 (m, 1H), 0.54-0.40 (m, 1H), 0.35-0.18 (m, 3H). MS (ESI⁺) m/z 550 (M+Na)⁺.

### Example 360

### N-(4-fluorobenzyl)-2-(3′-hydroxy-2,5-dioxo-2′,3′-dihydro-1H-spiro[imidazolidine-4,1′-inden]-1-yl)-N-[(2S)-1,1,1-trifluoropropan-2-yl]acetamide

The title compound was prepared as described in EXAMPLE 281F, substituting 3′-hydroxy-2′,3′-dihydrospiro[imidazolidine-4,1′-indene]-2,5-dione for (R)-5-bromo-2,3-dihydrospiro[indene-1,5′-oxazolidine]-2′,4′-dione. ¹H NMR (400 MHz, DMSO-d₆) δ 9.03-8.63 (m, 1H), 7.95 (s, 1H), 7.51-7.05 (m, 7H), 5.87-5.48 (m, 1H), 5.46-3.93 (m, 6H), 2.73 (s, 1H), 2.43-1.79 (m, 1H), 1.47-1.10 (m, 3H). MS (ESI⁺) m/z 480.0 (M+H)⁺.

### Example 361

### 2-{(1R)-2′,4′-dioxo-5-[(tetrahydrofuran-2-ylmethyl)amino]-2,3-dihydro-3′H-spiro[indene-1,5′-[1,3]oxazolidin]-3′-yl}-N-(4-fluorobenzyl)-N-[(2S)-1,1,1-trifluoropropan-2-yl]acetamide

### Example 361A

### 2-((R)-5-amino-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide

The title compound was prepared as described in EXAMPLE 284D-E, substituting 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide.

### Example 361B

### 2-{(1R)-2′,4′-dioxo-5-[(tetrahydrofuran-2-ylmethyl)amino]-2,3-dihydro-3′H-spiro[indene-,5′-[1,3]oxazolidin]-3′-yl}-N-(4-fluorobenzyl)-N-[(2S)-1,1,1-trifluoropropan-2-yl]acetamide

The title compound was prepared as described in EXAMPLE 283A, substituting 2-((R)-5-amino-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for (S)-1-cyclopropylethanamine and tetrahydrofuran-2-carbaldehyde for benzaldehyde. ¹H NMR (400 MHz, DMSO-d₆) δ ppm 7.36-7.27 (m, 2H), 7.20-7.11 (m, 2H), 7.07 (dd, J=8.9, 3.9 Hz, 1H), 6.58-6.53 (m, 2H), 4.86-4.79 (m, 1H), 4.73-4.47 (m, 2H), 3.97 (q, J=6.1 Hz, 1H), 3.83-3.74 (m, 1H), 3.64 (q, J=7.5 Hz, 1H), 3.12 (t, J=5.5 Hz, 2H), 3.05 (dd, J=15.5, 7.7 Hz, 1H), 2.93 (dt, J=14.5, 7.5 Hz, 1H), 2.62 (dt, J=15.2, 7.7 Hz, 1H), 2.43 (dq, J=11.5, 5.8, 4.8 Hz, 1H), 1.96 (ddd, J=14.9, 12.0, 6.5 Hz, 1H), 1.85 (dt, J=14.1, 7.7 Hz, 2H), 1.64-1.53 (m, 1H), 1.37 (d, J=7.1 Hz, 3H); MS (APCI⁺) m/z 564 (M+H)⁺.

### Example 362

### 2-{(1R)-5-[(cyclopentylmethyl)amino]-2′,4′-dioxo-2,3-dihydro-3′H-spiro[indene-1,5′-[1,3]oxazolidin]-3′-yl}-N-(4-fluorobenzyl)-N-[(2S)-1,1,1-trifluoropropan-2-yl]acetamide

The title compound was prepared as described in EXAMPLE 283A, substituting 2-((R)-5-amino-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for (S)-1-cyclopropylethanamine and cyclopentanecarbaldehyde for benzaldehyde. ¹H NMR (400 MHz, DMSO-d₆) δ ppm 7.37-7.28 (m, 2H), 7.16 (d, J=9.0 Hz, 2H), 7.06 (dd, J=9.0, 4.1 Hz, 1H), 6.55-6.48 (m, 2H), 5.27-5.07 (m, 1H), 4.83 (d, J=17.5 Hz, 1H), 4.73-4.47 (m, 2H), 4.44-4.23 (m, 1H), 3.05 (dt, J=15.6, 7.7 Hz, 1H), 2.96 (d, J=7.1 Hz, 2H), 2.94-2.86 (m, 1H), 2.61 (dt, J=15.2, 7.7 Hz, 1H), 2.46-2.38 (m, 1H), 2.13 (p, J=7.4 Hz, 1H), 1.74 (dq, J=12.1, 6.3 Hz, 2H), 1.65-1.45 (m, 5H), 1.37 (d, J=7.1 Hz, 3H), 1.24 (dq, J=13.6, 6.9 Hz, 2H); MS (APCI⁺) m/z 562 (M+H)⁺.

### Example 363

### 2-{(1R)-5-[(2,2-dimethylbutyl)amino]-2′,4′-dioxo-2,3-dihydro-3′H-spiro[indene-1,5′-[1,3]oxazolidin]-3′-yl}-N-(4-fluorobenzyl)-N-[(2S)-1,1,1-trifluoropropan-2-yl]acetamide

The title compound was prepared as described in EXAMPLE 283A, substituting 2-((R)-5-amino-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for (S)-1-cyclopropylethanamine and 2,2-dimethylbutanal for benzaldehyde. ¹H NMR (400 MHz, DMSO-d₆) δ ppm 7.37-7.25 (m, 2H), 7.15 (t, J=8.7 Hz, 2H), 7.08-7.01 (m, 1H), 6.64-6.53 (m, 2H), 5.28-5.08 (m, 1H), 4.83 (d, J=17.6 Hz, 1H), 4.74-4.46 (m, 2H), 4.43-4.26 (m, 1H), 3.05 (dt, J=15.5, 7.5 Hz, 1H), 2.91 (ddd, J=16.2, 8.7, 3.7 Hz, 1H), 2.86 (s, 2H), 2.61 (ddd, J=15.3, 8.8, 7.0 Hz, 1H), 2.42 (ddd, J=14.3, 8.2, 3.7 Hz, 1H), 1.37 (d, J=7.0 Hz, 3H), 1.31 (q, J=7.5 Hz, 2H), 0.89 (s, 6H), 0.82 (t, J=7.5 Hz, 3H); MS (APCI⁺) m/z 564 (M+H)⁺.

### Example 364

### 2-{(1R)-2′,4′-dioxo-5-[(tetrahydrofuran-3-ylmethyl)amino]-2,3-dihydro-3′H-spiro[indene-1,5′-[1,3]oxazolidin]-3′-yl}-N-(4-fluorobenzyl)-N-[(2S)-1,1,1-trifluoropropan-2-yl]acetamide

The title compound was prepared as described in EXAMPLE 283A, substituting 2-((R)-5-amino-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for (S)-1-cyclopropylethanamine and tetrahydrofuran-3-carbaldehyde for benzaldehyde. ¹H NMR (400 MHz, DMSO-d₆) δ ppm 7.39-7.28 (m, 2H), 7.19-7.05 (m, 3H), 6.55-6.49 (m, 2H), 4.83 (d, J=17.8 Hz, 1H), 3.78-3.72 (m, 2H), 3.68-3.60 (m, 1H), 3.45 (dd, J=8.5, 5.6 Hz, 1H), 3.04 (d, J=7.3 Hz, 2H), 2.94 (s, 1H), 2.67-2.57 (m, 1H), 2.49-2.38 (m, 1H), 2.04-1.94 (m, 1H), 1.68-1.55 (m, 1H), 1.37 (d, J=7.1 Hz, 3H); MS (APCI⁺) m/z 564 (M+H)⁺.

### Example 365

### N-(4-fluorobenzyl)-2-{(1R)-5-[(2-methylpropyl)amino]-2′,4′-dioxo-2,3-dihydro-3′H-spiro[indene-1,5′-[1,3]oxazolidin]-3′-yl}-N-[(2S)-1,1,1-trifluoropropan-2-yl]acetamide

The title compound was prepared as described in EXAMPLE 283A, substituting 2-((R)-5-amino-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for (S)-1-cyclopropylethanamine and isobutyraldehyde for benzaldehyde. MS (APCI⁺) m/z 536.1 (M+H)⁺.

### Example 366

### 2-[6-(acetylamino)-1,1-dioxido-2′,5′-dioxo-1′H-spiro[1-benzothiophene-3,4′-imidazolidin]-1′-yl]-N-[(1S)-1-cyclopropylethyl]-N-(4-fluorobenzyl)acetamide

The title compound was prepared as described in EXAMPLE 346, substituting 2-(6-amino-1,1-dioxido-2′,5′-dioxo-1′H-spiro[1-benzothiophene-3,4′-imidazolidin]-1′-yl)-N-[(1S)-1-cyclopropylethyl]-N-(4-fluorobenzyl)acetamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (400 MHz, DMSO-d₆, 90° C.) δ ppm 10.19 (brs, 1H), 8.97 (brs, 1H), 8.12 (d, J=2.0 Hz, 1H), 7.77 (dd, J=8.6, 1.9 Hz, 1H), 7.62 (d, J=8.6 Hz, 1H), 7.47-7.27 (m, 2H), 7.21-7.02 (m, 2H), 4.66 (brs, 2H), 4.50-4.20 (m, 2H), 3.96 (d, J=14.5 Hz, 1H), 3.79 (d, J=14.6 Hz, 1H), 3.71-3.29 (m, 1H), 2.09 (s, 3H), 1.20-1.10 (m, 3H), 1.00-0.89 (m, 1H), 0.54-0.44 (m, 1H), 0.37-0.18 (m, 3H). MS (ESI⁺) m/z 543 (M+H)⁺.

### Example 367

### tert-butyl [(2R)-1-{[3′-(2-{benzyl[(1S)-1-cyclopropylethyl]amino}-2-oxoethyl)-2′,4′-dioxo-2,3-dihydrospiro[chromene-4,5′-[1,3]oxazolidin]-7-yl]amino}-3-methyl-1-oxobutan-2-yl]carbamate

The title compound was prepared as described in EXAMPLE 306, substituting (R)-2-((tert-butoxycarbonyl)amino)-3-methylbutanoic acid for cyclopropanecarboxylic acid and 2-(7-amino-2′,4′-dioxo-2,3-dihydro-3′H-spiro[chromene-4,5′-[1,3]oxazolidin]-3′-yl)-N-benzyl-N-[(1S)-1-cyclopropylethyl]acetamide for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆, 90° C.) δ ppm 0.10-0.44 (m, 3H), 0.44-0.56 (m, 1H), 0.78-1.03 (m, 7H), 1.09-1.23 (m, 3H), 1.38 (s, 9H), 1.91-2.09 (m, 1H), 2.32-2.45 (m, 2H), 3.25-3.85 (m, 1H), 3.93 (t, J=7.8 Hz, 1H), 4.10-4.22 (m, 1H), 4.25-4.61 (m, 3H), 4.71 (brs, 2H), 6.26-6.36 (m, 1H), 7.07-7.14 m, 1H), 7.20-7.50 (m, 7H), 9.77 (s, 1H). MS (ESI⁺) m/z 671 (M+Na)⁺.

### Example 368

### N-[3′-(2-{benzyl[(1S)-1-cyclopropylethyl]amino}-2-oxoethyl)-2′,4′-dioxo-2,3-dihydrospiro[chromene-4,5′-[1,3]oxazolidin]-7-yl]-D-valinamide

The title compound was prepared as described in EXAMPLE 302, substituting tert-butyl [(2R)-1-{[3′-(2-{benzyl[(1S)-1-cyclopropylethyl]amino}-2-oxoethyl)-2′,4′-dioxo-2,3-dihydrospiro[chromene-4,5′-[1,3]oxazolidin]-7-yl]amino}-3-methyl-1-oxobutan-2-yl]carbamate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆, 90° C.) δ ppm 7.53-7.17 (m, 7H), 7.17-7.07 (m, 1H), 4.71 (brs, 2H), 4.58-4.25 (m, 3H), 4.21-4.09 (m, 1H), 3.89-3.28 (m, 1H), 3.13 (d, J=5.3 Hz, 1H), 2.47-2.33 (m, 2H), 2.05-1.85 (m, 1H), 1.18 (d, J=6.7 Hz, 3H), 1.05-0.94 (m, 1H), 0.93 (s, 3H), 0.86 (d, J=6.8 Hz, 3H), 0.57-0.45 (m, 1H), 0.34-0.15 (m, 3H). MS (ESI⁺) m/z 571 (M+Na)⁺.

### Example 369

### 2-[(1R)-6-amino-2′,4′-dioxo-2,3-dihydro-3′H-spiro[indene-1,5′-[1,3]oxazolidin]-3′-yl]-N-benzyl-N-[(1S)-1-cyclopropylethyl]acetamide

### Example 369A

### (R)-6-bromo-2,3-dihydrospiro[indene-1,5′-oxazolidine]-2′,4′-dione and (S)-6-bromo-2,3-dihydrospiro[indene-1,5′-oxazolidine]-2′,4′-dione

The title compounds were prepared as described in EXAMPLE 281C-E, substituting 6-bromo-2,3-dihydro-1H-inden-1-one for 5-bromo-2,3-dihydro-1H-inden-1-one in EXAMPLE 281C.

### Example 369B

### N-benzyl-2-((R)-6-bromo-2′,4′-dioxo-2,3-dihydrospiro[indene-1,5′-oxazolidin]-3′-yl)-N—((S)-1-cyclopropylethyl)acetamide

The title compound was prepared as described in EXAMPLE 281F, substituting (R)-6-bromo-2,3-dihydrospiro[indene-1,5′-oxazolidine]-2′,4′-dione for (R)-5-bromo-2,3-dihydrospiro[indene-1,5′-oxazolidine]-2′,4′-dione and (S)—N-benzyl-2-bromo-N-(1-cyclopropylethyl)acetamide for (S)-2-bromo-N-(4-fluorobenzyl)-N-(1,1,1-trifluoropropan-2-yl)acetamide.

### Example 369C

### 2-[(1R)-6-amino-2′,4′-dioxo-2,3-dihydro-3′H-spiro[indene-1,5′-[1,3]oxazolidin]-3′-yl]-N-benzyl-N-[(1S)-1-cyclopropylethyl]acetamide

The title compound was prepared as described in EXAMPLE 284D-E, substituting N-benzyl-2-((R)-6-bromo-2′,4′-dioxo-2,3-dihydrospiro[indene-1,5′-oxazolidin]-3′-yl)-N—((S)-1-cyclopropylethyl)acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide. ¹H NMR (400 MHz, DMSO-d₆, T=90° C.) δ 7.44-7.18 (m, 5H), 7.02 (d, J=8.1 Hz, 1H), 6.68 (dd, J=8.2, 2.1 Hz, 1H), 6.55 (d, J=1.9 Hz, 1H), 4.83 (s, 2H), 4.71 (s, 2H), 4.60-4.22 (m, 2H), 3.94-3.25 (m, 1H), 3.04-2.94 (m, 1H), 2.88 (ddd, J=15.5, 8.5, 4.7 Hz, 1H), 2.61 (ddd, J=14.3, 8.4, 6.0 Hz, 1H), 2.41 (ddd, J=13.6, 8.2, 4.6 Hz, 1H), 1.17 (d, J=6.9 Hz, 3H), 0.98 (ddt, J=13.4, 8.6, 4.3 Hz, 1H), 0.56-0.44 (m, 1H), 0.37-0.16 (m, 3H); MS (ESI⁺) m/z 434 (M+H)⁺.

### Example 370

### 2-[(1R)-6-(acetylamino)-2′,4′-dioxo-2,3-dihydro-3′H-spiro[indene-1,5′-[1,3]oxazolidin]-3′-yl]-N-benzyl-N-[(1S)-1-cyclopropylethyl]acetamide

The title compound was prepared as described in EXAMPLE 346, substituting 2-[(1R)-6-amino-2′,4′-dioxo-2,3-dihydro-3′H-spiro[indene-1,5′-[1,3]oxazolidin]-3′-yl]-N-benzyl-N-[(1S)-1-cyclopropylethyl]acetamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (400 MHz, DMSO-d₆, T=90° C.) δ 9.70 (s, 1H), 7.62 (dd, J=8.3, 2.0 Hz, 1H), 7.48 (d, J=2.0 Hz, 1H), 7.40-7.17 (m, 6H), 4.76-4.64 (m, 2H), 4.60-4.28 (m, 2H), 3.82-3.39 (m, 1H), 3.11 (ddd, J=16.3, 8.3, 5.8 Hz, 1H), 3.05-2.95 (m, 1H), 2.68 (ddd, J=14.2, 8.4, 5.8 Hz, 1H), 2.54-2.43 (m, 1H), 1.99 (s, 3H), 1.17 (d, J=7.0 Hz, 3H), 0.98 (ddt, J=13.4, 8.5, 4.2 Hz, 1H), 0.55-0.46 (m, 1H), 0.34-0.18 (m, 3H); MS (ESI⁺) m/z 434 (M+Na)⁺.

### Example 371

### N-benzyl-N-[(1S)-1-cyclopropylethyl]-2-{(1R)-6-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 282, substituting 2-[(1R)-6-amino-2′,4′-dioxo-2,3-dihydro-3′H-spiro[indene-1,5′-[1,3]oxazolidin]-3′-yl]-N-benzyl-N-[(1S)-1-cyclopropylethyl]acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆, T=90° C.) δ 8.28 (s, 1H), 7.49 (dd, J=8.3, 2.1 Hz, 1H), 7.40-7.18 (m, 7H), 5.87-5.79 (m, 1H), 4.76-4.64 (m, 2H), 4.58-4.26 (m, 2H), 3.88-3.35 (m, 1H), 3.08 (ddd, J=14.6, 8.3, 5.8 Hz, 1H), 2.98 (d, J=2.1 Hz, 1H), 2.70-2.60 (m, 4H), 2.51-2.43 (m, 1H), 1.17 (d, J=6.9 Hz, 3H), 0.98 (ddt, J=13.5, 8.4, 4.2 Hz, 1H), 0.55-0.45 (m, 1H), 0.34-0.19 (m, 3H); MS (ESI⁺) m/z 513 (M+Na)⁺.

### Example 372

### 2-[(1S)-6-amino-2′,4′-dioxo-2,3-dihydro-3′H-spiro[indene-1,5′-[1,3]oxazolidin]-3′-yl]-N-benzyl-N-[(1S)-1-cyclopropylethyl]acetamide

### Example 372A

### N-benzyl-2-((S)-6-bromo-2′,4′-dioxo-2,3-dihydrospiro[indene-1,5′-oxazolidin]-3′-yl)-N—((S)-1-cyclopropylethyl)acetamide

The title compound was prepared as described in EXAMPLE 281F, substituting (S)-6-bromo-2,3-dihydrospiro[indene-1,5′-oxazolidine]-2′,4′-dione for (R)-5-bromo-2,3-dihydrospiro[indene-1,5′-oxazolidine]-2′,4′-dione and (S)—N-benzyl-2-bromo-N-(1-cyclopropylethyl)acetamide for (S)-2-bromo-N-(4-fluorobenzyl)-N-(1,1,1-trifluoropropan-2-yl)acetamide.

### Example 372B

### 2-[(1S)-6-amino-2′,4′-dioxo-2,3-dihydro-3′H-spiro[indene-1,5′-[1,3]oxazolidin]-3′-yl]-N-benzyl-N-[(1S)-1-cyclopropylethyl]acetamide

The title compound was prepared as described in EXAMPLE 284D-E, substituting N-benzyl-2-((S)-6-bromo-2′,4′-dioxo-2,3-dihydrospiro[indene-1,5′-oxazolidin]-3′-yl)-N—((S)-1-cyclopropylethyl)acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide. ¹H NMR (400 MHz, DMSO-d₆, T=90° C.) δ 7.28 (d, J=39.3 Hz, 5H), 7.02 (d, J=8.2 Hz, 1H), 6.68 (dd, J=8.1, 2.2 Hz, 1H), 6.56 (d, J=2.1 Hz, 1H), 4.82 (s, 2H), 4.70 (s, 2H), 4.59-4.24 (m, 2H), 3.87-3.29 (m, 1H), 3.04-2.93 (m, 1H), 2.88 (ddd, J=15.5, 8.4, 4.7 Hz, 1H), 2.61 (ddd, J=14.3, 8.5, 6.0 Hz, 1H), 2.41 (ddd, J=14.3, 8.2, 4.6 Hz, 1H), 1.17 (d, J=7.2 Hz, 3H), 0.97 (ddt, J=13.5, 8.4, 4.1 Hz, 1H), 0.55-0.45 (m, 1H), 0.32-0.18 (m, 3H); MS (ESI⁺) m/z 433 (M+H)⁺.

### Example 373

### 2-[(1S)-6-(acetylamino)-2′,4′-dioxo-2,3-dihydro-3′H-spiro[indene-1,5′-[1,3]oxazolidin]-3′-yl]-N-benzyl-N-[(1S)-1-cyclopropylethyl]acetamide

The title compound was prepared as described in EXAMPLE 346, substituting 2-[(1S)-6-amino-2′,4′-dioxo-2,3-dihydro-3′H-spiro[indene-1,5′-[1,3]oxazolidin]-3′-yl]-N-benzyl-N-[(1S)-1-cyclopropylethyl]acetamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (400 MHz, DMSO-d₆, T 10=90° C.) δ 9.70 (s, 1H), 7.61 (dd, J=8.2, 2.0 Hz, 1H), 7.49 (d, J=2.1 Hz, 1H), 7.44-7.16 (m, 6H), 4.70 (s, 2H), 4.54-4.25 (m, 2H), 3.86-3.33 (m, 1H), 3.15-2.97 (m, 2H), 2.68 (ddd, J=14.1, 8.4, 5.7 Hz, 1H), 2.53-2.43 (m, 1H), 1.99 (s, 3H), 1.19-1.15 (m, 3H), 0.97 (ddt, J=13.5, 8.5, 4.3 Hz, 1H), 0.54-0.45 (m, 1H), 0.34-0.17 (m, 3H); MS (ESI⁺) m/z 498 (M+Na)⁺.

### Example 374

### N-benzyl-N-[(1S)-1-cyclopropylethyl]-2-{(1S)-6-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 282, substituting 2-[(1S)-6-amino-2′,4′-dioxo-2,3-dihydro-3′H-spiro[indene-1,5′-[1,3]oxazolidin]-3′-yl]-N-benzyl-N-[(1S)-1-cyclopropylethyl]acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆, T=90° C.) δ 8.27 (s, 1H), 7.48 (dd, J=8.3, 2.1 Hz, 1H), 7.42-7.18 (m, 7H), 5.90-5.74 (m, 1H), 4.70 (s, 2H), 4.58-4.26 (m, 2H), 3.82-3.34 (m, 1H), 3.13-2.93 (m, 2H), 2.71-2.60 (m, 4H), 2.51-2.42 (m, 1H), 1.17 (d, J=6.7 Hz, 3H), 0.98 (ddt, J=11.8, 8.1, 4.2 Hz, 1H), 0.54-0.45 (m, 1H), 0.33-0.19 (m, 3H); MS (ESI⁺) m/z 513 (M+Na)⁺.

### Example 375

### N-[(1S)-1-cyclopropylethyl]-2-[(1R)-2′,4′-dioxo-5-(pyridin-2-yl)-2,3-dihydro-3′H-spiro[indene-1,5′-[1,3]oxazolidin]-3′-yl]-N-(4-fluorobenzyl)acetamide

The title compound was prepared as described in EXAMPLE 295, substituting 2-bromopyridine for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N—((S)-1-cyclopropylethyl)-2-((R)-2′,4′-dioxo-5-(4,4,5,5-tetramethyl-1,3,2-dioxaborolan-2-yl)-2,3-dihydrospiro[indene-1,5′-oxazolidin]-3′-yl)-N-(4-fluorobenzyl)acetamide for 5-(4,4,5,5-tetramethyl-1,3,2-dioxaborolan-2-yl)pyridin-2-ol. ¹H NMR (400 MHz, DMSO-d₆, 90° C.) δ ppm 8.66 (dt, J=4.8, 1.4 Hz, 1H), 8.08 (d, J=1.6 Hz, 1H), 8.00 (dd, J=8.0, 1.6 Hz, 1H), 7.96-7.90 (m, 1H), 7.86 (td, J=7.7, 1.8 Hz, 1H), 7.56 (d, J=8.1 Hz, 1H), 7.48-7.29 (m, 3H), 7.21-7.00 (m, 2H), 4.69 (brs, 2H), 4.63-4.26 (m, 2H), 3.87-3.32 (m, 1H), 3.32-3.08 (m, 2H), 2.83-2.67 (m, 1H), 2.65-2.52 (m, 1H), 1.19 (d, J=6.7 Hz, 3H), 1.07-0.91 (m, 1H), 0.58-0.47 (m, 1H), 0.38-0.19 (m, 3H). MS (ESI⁺) m/z 514 (M+H)⁺.

### Example 376

### 2-(7′-bromo-2,5-dioxo-3′,4′-dihydro-1H, 1′H-spiro[imidazolidine-4,2′-naphthalen]-1-yl)-N-[(1S)-1-cyclopropylethyl]-N-(4-fluorobenzyl)acetamide

The title compound was prepared as described in EXAMPLE 290B-C, substituting 7-bromo-3,4-dihydronaphthalen-2(1H)-one for 6-bromo-2H-spiro[benzo[b]thiophene-3,4′-imidazolidine]-2′,5′-dione 1,1-dioxide in EXAMPLE 290B. ¹H NMR (400 MHz, DMSO-d₆, 90° C.) 6 ppm 8.29 (brs, 1H), 7.54-7.23 (m, 4H), 7.15-7.03 (m, 3H), 4.63 (brs, 2H), 4.40-4.08 (m, 2H), 3.78-3.20 (m, 1H), 3.15 (d, J=17.0 Hz, 1H), 2.94-2.86 (m, 2H), 2.82 (d, J=17.0 Hz, 1H), 2.10-1.93 (m, 1H), 1.93-1.79 (m, 1H), 1.15 (d, J=6.8 Hz, 3H), 1.03-0.87 (m, 1H), 0.53-0.41 (m, 1H), 0.35-0.14 (m, 3H). MS (ESI⁺) m/z 550 (M+Na)⁺.

### Example 377

### N-[(1S)-1-cyclopropylethyl]-N-(3-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

### Example 377A

### (R)-tert-butyl 2-(5-bromo-2′,4′-dioxo-2,3-dihydrospiro[indene-1,5′-oxazolidin]-3′-yl)acetate

The title compound was prepared as described in EXAMPLE 281F, substituting tert-butyl bromoacetate for (S)-2-bromo-N-(4-fluorobenzyl)-N-(1,1,1-trifluoropropan-2-yl)acetamide.

### Example 377B

### (R)-tert-butyl 2-(5-amino-2′,4′-dioxo-2,3-dihydrospiro[indene-1,5′-oxazolidin]-3′-yl)acetate

The title compound was prepared as described in EXAMPLE 284D-E, substituting (R)-tert-butyl 2-(5-bromo-2′,4′-dioxo-2,3-dihydrospiro[indene-1,5′-oxazolidin]-3′-yl)acetate for N-benzyl-2-((S)-5-bromo-2′,4′-dioxo-2,3-dihydrospiro[indene-1,5′-oxazolidin]-3′-yl)-N—((S)-1-cyclopropylethyl)acetamide.

### Example 377C

### (R)-tert-butyl 2-(5-(3-methylureido)-2′,4′-dioxo-2,3-dihydrospiro[indene-1,5′-oxazolidin]-3′-yl)acetate

The title compound was prepared as described in EXAMPLE 282, substituting (R)-tert-butyl 2-(5-amino-2′,4′-dioxo-2,3-dihydrospiro[indene-1,5′-oxazolidin]-3′-yl)acetate for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride.

### Example 377D

### (R)-2-(5-(3-methylureido)-2′,4′-dioxo-2,3-dihydrospiro[indene-1,5′-oxazolidin]-3′-yl)acetic acid

The title compound was prepared as described in EXAMPLE 302, substituting (R)-tert-butyl 2-(5-(3-methylureido)-2′,4′-dioxo-2,3-dihydrospiro[indene-1,5′-oxazolidin]-3′-yl)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 377E

### (S)-1-cyclopropyl-N-(3-fluorobenzyl)ethanamine

The title compound was prepared as described in EXAMPLE 283A, substituting 3-fluorobenzaldehyde for benzaldehyde.

### Example 377F

### N-[(1S)-1-cyclopropylethyl]-N-(3-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

A mixture of (R)-2-(5-(3-methylureido)-2′,4′-dioxo-2,3-dihydrospiro[indene-1,5′-oxazolidin]-3′-yl)acetic acid (80 mg, 0.240 mmol), (S)-1-cyclopropyl-N-(3-fluorobenzyl)ethanamine (46.4 mg, 0.240 mmol), 2-(3H-[1,2,3]triazolo[4,5-b]pyridin-3-yl)-1,1,3,3-tetramethylisouronium hexafluorophosphate(V) (110 mg, 0.288 mmol), and N-ethyl-N-isopropylpropan-2-amine (0.126 ml, 0.720 mmol) in N,N-dimethylformamide (1.5 ml) was stirred at room temperature for 24 hours. The crude reaction mixture was directly purified by reverse phase HPLC to provide the title compound. ¹H NMR (400 MHz, DMSO-d₆) δ 8.69 (s, 1H), 7.69-6.88 (m, 7H), 6.20-5.96 (m, 1H), 4.93-3.65 (m, 5H), 3.23-2.87 (m, 2H), 2.71-2.39 (m, 5H), 1.18 (dd, J=32.3, 6.6 Hz, 3H), 1.08-0.85 (m, 1H), 0.61-0.08 (m, 4H). MS (ESI) m/e 531 (M+Na)⁺.

TABLE 16
The following EXAMPLEs were prepared essentially as described in EXAMPLE 377, substituting
the appropriate amine and appropriate aldehyde in EXAMPLE 377E. In some cases, racemic
5-bromo-2,3-dihydrospiro[indene-1,5′-oxazolidine]-2′,4′-dione was substituted
for (R)-5-bromo-2,3-dihydrospiro[indene-1,5′-oxazolidine]-2′,4′-dione in
EXAMPLE 377A. Some products were purified by flash chromatography while others were purified
by reverse-phase HPLC. Accordingly, some EXAMPLEs were isolated as trifluoroacetic acid salts.

| Ex | Name | NMR | MS |
|---|---|---|---|
| 378 | N-[(1S)-1- | ¹H NMR (400 MHz, DMSO-d₆) δ 8.69 (s, | (ESI(+)) |
| | cyclopropylethyl]-N-(3,4- | 1H), 7.72-6.79 (m, 6H), 6.26-5.79 (m, | m/e 549 |
| | difluorobenzyl)-2-{(1R)-5- | 1H), 4.90-3.62 (m, 5H), 3.22-2.86 (m, | (M + Na)⁺ |
| | [(methylcarbamoyl)amino]- | 2H), 2.76-2.34 (m, 5H), 1.19 (dd, J = 36.1, | |
| | 2′,4′-dioxo-2,3-dihydro-3′H- | 6.6 Hz, 3H), 1.06-0.78 (m, 1H), 0.64-0.10 | |
| | spiro[indene-1,5′- | (m, 4H) | |
| | [1,3]oxazoridin]-3′- | | |
| | yl}acetamide | | |
| 393 | N-[(1S)-1- | ¹H NMR (400 MHz, DMSO-d₆) δ 8.83- | (ESI(+)) |
| | cyclopropylethyl]-N-(2- | 8.41 (m, 1H), 7.82-6.81 (m, 7H), 6.24- | m/e 531 |
| | fluorobenzyl)-2-{(1R)-5- | 5.67 (m, 1H), 4.95-3.58 (m, 5H), 3.24- | (M + Na)⁺ |
| | [(methylcarbamoyl)amino]- | 2.86 (m, 2H), 2.75-2.29 (m, 5H), 1.18 (dd, | |
| | 2′,4′-dioxo-2,3-dihydro-3′H- | J = 45.4, 6.6 Hz, 3H), 1.06-0.80 (m, 1H), | |
| | spiro[indene-1,5′- | 0.71-0.05 (m, 4H) | |
| | [1,3]oxazolidin]-3′- | | |
| | yl}acetamide | | |
| 394 | N-[(1S)-1- | ¹H NMR (400 MHz, DMSO-d₆) δ 8.69 (s, | (ESI(+)) |
| | cyclopropylethyl]-N-(2,4- | 1H), 7.66-6.73 (m, 6H), 6.22-5.94 (m, | m/e 549 |
| | difluorobenzyl)-2-{(1R)-5- | 1H), 4.85-3.49 (m, 5H), 3.25-2.90 (m, | (M + Na)⁺ |
| | [(methylcarbamoyl)amino]- | 2H), 2.82-2.36 (m, 5H), 1.18 (dd, J = 46.8, | |
| | 2′,4′-dioxo-2,3-dihydro-3′H- | 6.6 Hz, 3H), 1.07-0.84 (m, 1H), 0.68-0.04 | |
| | spiro[indene-1,5′- | (m, 4H) | |
| | [1,3]oxazolidin]-3′- | | |
| | yl}acetamide | | |
| 395 | N-[(1S)-1- | ¹H NMR (400 MHz, DMSO-d₆) δ 8.81- | (ESI(+)) |
| | cyclopropylethyl]-N-(2,3- | 8.44 (m, 1H), 7.95-6.66 (m, 6H), 6.27- | m/e 549 |
| | difluorobenzyl)-2-{(1R)-5- | 5.89 (m, 1H), 4.93-3.35 (m, 5H), 3.20- | (M + Na)⁺ |
| | [(methylcarbamoyl)amino]- | 2.92 (m, 2H). 2.77-2.36 (m, 5H), 1.19 (dd, | |
| | 2′,4′-dioxo-2,3-dihydro-3′H- | J = 48.4, 6.6 Hz, 3H), 1.09-0.81 (m, 1H), | |
| | spiro[indene-1,5′- | 0.66-0.10 (m, 4H) | |
| | [1,3]oxazolidin]-3′- | | |
| | yl}acetamide | | |
| 396 | N-[(1S)-1- | ¹H NMR (400 MHz, DMSO-d₆) δ 8.80- | (ESI(+)) |
| | cyclopropylethyl]-N-(2,5- | 8.55 (m, 1H), 7.74-6.76 (m, 6H), 6.19- | m/e 549 |
| | difluorobenzyl)-2-{(1R)-5- | 5.90 (m, 1H), 4.89-3.41 (m, 5H), 3.19- | (M + Na)⁺ |
| | [(methylcarbamoyl)amino]- | 2.92 (m, 2H), 2.75-2.35 (m, 5H), 1.20 (dd, | |
| | 2′,4′-dioxo-2,3-dihydro-3′H- | J = 49.9, 6.6 Hz, 3H), 1.06-0.82 (m, 1H), | |
| | spiro[indene-1,5′- | 0.63-0.06 (m, 4H) | |
| | [1,3]oxazolidin]-3′- | | |
| | yl}acetamide | | |
| 397 | N-(2-chloro-4- | ¹H NMR (400 MHz, DMSO-d₆) δ 8.75- | (ESI(+)) |
| | fluorobenzyl)-N-[(1S)-1- | 8.49 (m, 1H), 7.69-7.03 (m, 6H), 6.25- | m/e 565 |
| | cyclopropylethyl]-2-{(1R)- | 5.79 (m, 1H), 4.93-3.40 (m, 5H), 3.24- | (M + Na)⁺ |
| | 5-[(methylcarbamoyl)amino]- | 2.87 (m, 2H), 2.78-2.37 (m, 5H), 1.19 (dd, | |
| | 2′,4′-dioxo-2,3-dihydro-3′H- | J = 45.7, 6.6 Hz, 3H), 1.01-0.72 (m, 1H), | |
| | spiro[indene-1,5′- | 0.59-0.09 (m, 4H) | |
| | [1,3]oxazolidin]-3′- | | |
| | yl}acetamide | | |
| 398 | N-(3-chloro-4- | ¹H NMR (400 MHz, DMSO-d₆) δ 8.69 (s, | (ESI(+)) |
| | fluorobenzyl)-N-[(1S)-1- | 1H), 7.82-6.89 (m, 6H), 6.29-5.75 (m, | m/e 565 |
| | cyclopropylethyl]-2-{(1R)- | 1H), 4.98-3.28 (m, 5H), 3.23-2.87 (m, | (M + Na)⁺ |
| | 5-[(methylcarbamoyl)amino]- | 2H), 2.77-2.36 (m, 5H), 1.19 (dd, J = 36.7, | |
| | 2′,4′-dioxo-2,3-dihydro-3′H- | 6.6 Hz, 3H), 1.08-0.77 (m, 1H), 0.64-0.10 | |
| | spiro[indene-1,5′- | (m, 4H) | |
| | [1,3]oxazolidin]-3′- | | |
| | yl}acetamide | | |
| 399 | N-(4-chlorobenzyl)-N-[(1S)- | ¹H NMR (501 MHz, DMSO-d₆) δ 8.68 (s, | (ESI(+)) |
| | 1-cyclopropylethyl]-2- | 1H), 7.66-7.05 (m, 7H), 6.18-5.88 (m, | m/e 547 |
| | {(1R)-5- | 1H), 4.89-3.28 (m, 5H), 3.22-2.89 (m, | (M + Na)⁺ |
| | [(methylcarbamoyl)amino]- | 2H), 2.74-2.32 (m, 5H), 1.18 (dd, J = 37.9, | |
| | 2′,4′-dioxo-2,3-dihydro-3′H- | 6.7 Hz, 3H), 1.09-0.81 (m, 1H), 0.69-0.06 | |
| | spiro[indene-1,5′- | (m, 4H) | |
| | [1,3]oxazolidin]-3′- | | |
| | yl}acetamide | | |
| 400 | N-[(1S)-1- | ¹H NMR (501 MHz, DMSO-d₆) δ 8.82- | (ESI(+)) |
| | cyclopropylethyl]-2-{(1R)- | 8.55 (m, 1H), 7.88-7.02 (m, 7H), 6.32- | m/e 581 |
| | 5-[(methylcarbamoyl)amino]- | 5.68 (m, 1H), 4.98-3.29 (m, 5H), 3.21- | (M + Na)⁺ |
| | 2′,4′-dioxo-2,3-dihydro-3′H- | 2.87 (m, 2H), 2.77-2.33 (m, 5H), 1.20 (dd, | |
| | spiro[indene-1,5′- | J = 44.7, 6.6 Hz, 3H), 1.07-0.85 (m, 1H), | |
| | [1,3]oxazolidin]-3′-yl}-N-[4- | 0.64-0.11 (m, 4H) | |
| | (trifluoromethyl)benzyl]acetamide | | |
| 409 | N-[(1S)-1- | ¹H NMR (400 MHz, DMSO-d₆) δ 8.80- | (ESI(+)) |
| | cyclopropylethyl]-2-{(1R)- | 8.56 (m, 1H), 7.54 (d, J = 2.2 Hz, 1H), 7.37- | m/e 535 |
| | 5-[(methylcarbamoyl)amino]- | 7.15 (m, 2H), 6.28-5.78 (m, 1H), 5.31- | (M + Na)⁺ |
| | 2′,4′-dioxo-2,3-dihydro-3′H- | 3.24 (m, 5H), 3.23-2.91 (m, 2H), 2.82- | |
| | spiro[indene-1,5′- | 2.36 (m, 8H), 1.47-0.96 (m, 4H), 0.72- | |
| | [1,3]oxazolidin]-3′-yl}-N- | 0.07 (m, 4H) | |
| | [(5-methyl-1,3,4-thiadiazol- | | |
| | 2-yl)methyl]acetamide | | |
| 413 | N-(4-cyanobenzyl)-N-[(1S)- | ¹H NMR (400 MHz, DMSO-d₆) δ 8.84- | (ESI(+)) |
| | 1-cyclopropylethyl]-2- | 8.51 (m, 1H), 8.04-6.91 (m, 7H), 6.21- | m/e 538 |
| | {(1R)-5- | 5.80 (m, 1H), 5.06-3.33 (m, 5H), 3.22- | (M + Na)⁺ |
| | [(methylcarbamoyl)amino]- | 2.84 (m, 2H), 2.75-2.32 (m, 5H), 1.19 (dd, | |
| | 2′,4′-dioxo-2,3-dihydro-3′H- | J = 38.2, 6.6 Hz, 3H), 1.04-0.78 (m, 1H), | |
| | spiro[indene-1,5′- | 0.59-0.04 (m, 4H) | |
| | [1,3]oxazolidin]-3′- | | |
| | yl}acetamide | | |
| 414 | N-[(1S)-1- | ¹H NMR (400 MHz, DMSO-d₆) δ 8.68 (s, | (ESI(+)) |
| | cyclopropylethyl]-N-(3- | 1H), 7.64-6.83 (m, 7H), 6.21-5.85 (m, | m/e 527 |
| | methylbenzyl)-2-{(1R)-5- | 1H), 4.83-3.33 (m, 5H), 3.21-2.85 (m, | (M + Na)⁺ |
| | [(methylcarbamoyl)amino]- | 2H), 2.71-2.41 (m, 5H), 2.28 (d, J = 22.6 | |
| | 2′,4′-dioxo-2,3-dihydro-3′H- | Hz, 3H), 1.16 (dd, J = 24.0, 6.7 Hz, 3H), | |
| | spiro[indene-1,5′- | 1.05-0.81 (m, 1H), 0.65-0.08 (m, 4H) | |
| | [1,3]oxazolidin]-3′- | | |
| | yl}acetamide | | |
| 415 | N-[(1S)-1- | ¹H NMR (400 MHz, DMSO-d₆) δ 8.78- | (ESI(+)) |
| | cyclopropylethyl]-N-(3,5- | 8.60 (m, 1H), 7.66-6.79 (m, 6H), 6.24- | m/e 549 |
| | difluorobenzyl)-2-{(1R)-5- | 5.93 (m, 1H), 4.91-3.38 (m, 5H), 3.20- | (M + Na)⁺ |
| | [(methylcarbamoyl)amino]- | 2.89 (m, 2H), 2.72-2.34 (m, 5H), 1.19 (dd, | |
| | 2′,4′-dioxo-2,3-dihydro-3′H- | J = 39.4, 6.6 Hz, 3H), 1.05-0.83 (m, 1H), | |
| | spiro[indene-1,5′- | 0.61-0.07 (m, 4H) | |
| | [1,3]oxazolidin]-3′- | | |
| | yl}acetamide | | |
| 416 | N-(3-chlorobenzyl)-N-[(1S)- | ¹H NMR (400 MHz, DMSO-d₆) δ 8.69 (s, | (ESI(+)) |
| | 1-cyclopropylethyl]-2- | 1H), 7.67-7.08 (m, 7H), 6.16-5.87 (m, | m/e 547 |
| | {(1R)-5- | 1H), 4.89-3.36 (m, 5H), 3.21-2.90 (m, | (M + Na)⁺ |
| | [(methylcarbamoyl)amino]- | 2H), 2.75-2.29 (m, 5H), 1.18 (dd, J = 33.7, | |
| | 2′,4′-dioxo-2,3-dihydro-3′H- | 6.6 Hz, 3H), 1.07-0.80 (m, 1H), 0.66-0.08 | |
| | spiro[indene-1,5′- | (m, 4H) | |
| | [1,3]oxazolidin]-3′- | | |
| | yl}acetamide | | |
| 417 | N-(2-chlorobenzyl)-N-[(1S)- | ¹H NMR (400 MHz, DMSO-d₆) δ 8.77- | (ESI(+)) |
| | 1-cyclopropylethyl]-2- | 8.56 (m, 1H), 7.66-6.99 (m, 7H), 6.23- | m/e 547 |
| | {(1R)-5- | 5.89 (m, 1H), 4.92-3.41 (m, 5H), 3.20- | (M + Na)⁺ |
| | [(methylcarbamoyl)amino]- | 2.88 (m, 2H), 2.74-2.32 (m, 5H), 1.19 (dd, | |
| | 2′,4′-dioxo-2,3-dihydro-3′H- | J = 45.2, 6.6 Hz, 3H), 1.01-0.75 (m, 1H), | |
| | spiro[indene-1,5′- | 0.61-0.10 (m, 4H) | |
| | [1,3]oxazoridin]-3′- | | |
| | yl}acetamide | | |
| 418 | N-[(1S)-1- | ¹H NMR (400 MHz, DMSO-d₆) δ 8.75- | (ESI(+)) |
| | cyclopropylethyl]-N-(2,4- | 8.59 (m, 1H), 7.82-7.05 (m, 6H), 6.18- | m/e 581 |
| | dichlorobenzyl)-2-{(1R)-5- | 5.93 (m, 1H), 4.90-3.41 (m, 5H), 3.21- | (M + Na)⁺ |
| | [(methylcarbamoyl)amino]- | 2.91 (m, 2H), 2.80-2.34 (m, 5H), 1.19 (dd, | |
| | 2′,4′-dioxo-2,3-dihydro-3′H- | J = 47.6, 6.6 Hz, 3H), 1.02-0.74 (m, 1H), | |
| | spiro[indene-1,5′- | 0.63-0.12 (m, 4H) | |
| | [1,3]oxazoridin]-3′- | | |
| | yl}acetamide | | |
| 419 | N-[(1S)-1- | ¹H NMR (400 MHz, DMSO-d₆) δ 8.69 (s, | (ESI(+)) |
| | cyclopropylethyl]-N-(4- | 1H), 7.60-6.77 (m, 7H), 6.20-5.91 (m, | m/e 543 |
| | methoxybenzyl)-2-{(1R)-5- | 1H), 4.81-4.21 (m, 4H), 3.90-3.58 (m, | (M + Na)⁺ |
| | [(methylcarbamoyl)amino]- | 4H), 3.22-2.83 (m, 2H), 2.75-2.35 (m, | |
| | 2′,4′-dioxo-2,3-dihydro-3′H- | 5H), 1.16 (dd, J = 20.3, 6.7 Hz, 3H), 0.97 (m, | |
| | spiro[indene-1,5′- | 1H), 0.61-0.10 (m, 4H) | |
| | [1,3]oxazoridin]-3′- | | |
| | yl}acetamide | | |
| 420 | N-[(1S)-1- | ¹H NMR (400 MHz, DMSO-d₆) δ 8.69 (s, | (ESI(+)) |
| | cyclopropylethyl]-N-(3- | 1H), 7.70-6.62 (m, 7H), 6.21-5.80 (m, | m/e 543 |
| | methoxybenzyl)-2-{(1R)-5- | 1H), 4.87-4.20 (m, 4H), 3.85-3.64 (m, | (M + Na)⁺ |
| | [(methylcarbamoyl)amino]- | 4H), 3.25-2.86 (m, 2H), 2.75-2.29 (m, | |
| | 2′,4′-dioxo-2,3-dihydro-3′H- | 5H), 1.17 (dd, J = 23.3, 6.6 Hz, 3H), 1.05- | |
| | spiro[indene-1,5′- | 0.87 (m, 1H), 0.59-0.12 (m, 4H) | |
| | [1,3]oxazolidin]-3′- | | |
| | yl}acetamide | | |
| 421 | N-[(1S)-1- | ¹H NMR (400 MHz, DMSO-d₆) δ 8.68 (s, | (ESI(+)) |
| | cyclopropylethyl]-N-(4- | 1H), 7.62-6.94 (m, 7H), 6.19-5.88 (m, | m/e 527 |
| | methylbenzyl)-2-{(1R)-5- | 1H), 4.83-3.34 (m, 5H), 3.23-2.89 (m, | (M + Na)⁺ |
| | [(methylcarbamoyl)amino]- | 2H), 2.72-2.40 (m, 5H), 2.27 (d, J = 14.2 | |
| | 2′,4′-dioxo-2,3-dihydro-3′H- | Hz, 3H), 1.15 (dd, J = 21.4, 6.7 Hz, 3H), | |
| | spiro[indene-1,5′- | 1.04-0.88 (m, 1H), 0.59-0.10 (m, 4H) | |
| | [1,3]oxazolidin]-3′- | | |
| | yl}acetamide | | |
| 496 | N-[(1S)-1- | ¹H NMR (400 MHz, DMSO-d₆) δ 9.28- | (ESI(+)) |
| | cyclopropylethyl]-2-{(1R)- | 8.47 (m, 4H), 7.66-6.87 (m, 3H), 6.24- | m/e 493 |
| | 5- | 5.95 (m, 1H), 5.04-3.38 (m, 5H), 3.21- | (M + H)⁺ |
| | [(methylcarbamoyl)amino]- | 2.83 (m, 2H), 2.79-2.30 (m, 5H), 1.23 (dd, | |
| | 2′,4′-dioxo-2,3-dihydro-3′H- | J = 54.7, 6.6 Hz, 3H), 1.10-0.75 (m, 1H), | |
| | spiro[indene-1,5′- | 0.64-−0.00 (m, 4H) | |
| | [1,3]oxazolidin]-3′-yl}-N- | | |
| | (pyrimidin-5- | | |
| | ylmethyl)acetamide | | |
| 497 | N-[(1S)-1- | ¹H NMR (400 MHz, DMSO-d₆) δ 8.93- | (ESI(+)) |
| | cyclopropylethyl]-2-{(1R)- | 8.57 (m, 3H), 7.65-7.06 (m, 4H), 6.27- | m/e 493 |
| | 5-[(methylcarbamoyl)amino]- | 5.89 (m, 1H), 5.07-3.45 (m, 5H), 3.22- | (M + H)⁺ |
| | 2′,4′-dioxo-2,3-dihydro-3′H- | 2.89 (m, 2H), 2.79-2.39 (m, 5H), 1.12 (dd, | |
| | spiro[indene-1,5′- | J = 99.9, 6.6 Hz, 3H), 0.96-0.79 (m, 1H), | |
| | [1,3]oxazolidin]-3′-yl}-N- | 0.58-−0.07 (m, 4H) | |
| | (pyrimidin-2-ylmethyl)acetamide | | |
| 498 | N-[(1S)-1- | ¹H NMR (400 MHz, DMSO-d₆) δ 9.27- | (ESI(+)) |
| | cyclopropylethyl]-2-{(1R)- | 8.44 (m, 2H), 8.02-7.06 (m, 4H), 6.27- | m/e 498 |
| | 5-[(methylcarbamoyl)amino]- | 5.83 (m, 1H), 5.18-3.24 (m, 5H), 3.22- | (M + H)⁺ |
| | 2′,4′-dioxo-2,3-dihydro-3′H- | 2.89 (m, 2H), 2.77-2.43 (m, 5H), 1.38- | |
| | spiro[indene-1,5′- | 1.01 (m, 4H), 0.67-0.05 (m, 4H) | |
| | [1,3]oxazolidin]-3′-yl}-N- | | |
| | (1,3-thiazol-5-ylmethyl)acetamide | | |
| 512 | N-benzyl-N-ethyl-2-{(1R)- | ¹H NMR (400 MHz, DMSO-d₆, | (ESI(+)) |
| | 5-[(methylcarbamoyl)amino]- | Temp = 120° C.) δ ppm 8.08 (s, 3H), 7.31 (d, J = | m/e 901 |
| | 2′,4′-dioxo-2,3-dihydro-3′H- | 1.7 Hz, 3H), 7.22-7.01 (m, 19H), 5.66 (s, | (2M + H)⁺ |
| | spiro[indene-1,5′- | 2H), 4.29 (d, J = 2.7 Hz, 6H), 3.22 (q, J = 7.1 | |
| | [1,3]oxazolidin]-3′- | Hz, 6H), 2.95 (dt, J = 15.3, 7.4 Hz, 3H), 0.93 | |
| | yl}acetamide | (t, J = 7.1 Hz, 9H) | |
| 520 | N-[(1S)-1- | ¹H NMR (400 MHz, DMSO-d₆) δ 8.69 (d, J = | (ESI(+)) |
| | cyclopropylethyl]-2-{(1R)- | 2.6 Hz, 1H), 7.54 (d, J = 1.8 Hz, 1H), 7.33- | m/e 499 |
| | 5-[(methylcarbamoyl)amino]- | 7.18 (m, 2H), 6.17-5.92 (m, 1H), 4.62- | (M + H)⁺ |
| | 2′,4′-dioxo-2,3-dihydro-3′H- | 2.79 (m, 13H), 2.64 (s, 3H), 2.05-0.94 (m, | |
| | spiro[indene-1,5′- | 9H), 0.70-0.08 (m, 4H) | |
| | [1,3]oxazolidin]-3′-yl}-N- | | |
| | (tetrahydro-2H-pyran-4- | | |
| | ylmethyl)acetamide | | |
| 521 | N-[(1S)-1- | ¹H NMR (400 MHz, DMSO-d₆) δ 8.63 (d, J = | (ESI(+)) |
| | cyclopropylethyl]-2-{(1R)- | 2.7 Hz, 1H), 8.39-7.38 (m, 3H), 7.29- | m/e 482 |
| | 5-[(methylcarbamoyl)amino]- | 7.08 (m, 2H), 6.15-5.76 (m, 1H), 4.80- | (M + H)⁺ |
| | 2′,4′-dioxo-2,3-dihydro-3′H- | 3.22 (m, 5H), 3.14-2.82 (m, 2H), 2.66- | |
| | spiro[indene-1,5′- | 2.32 (m, 5H), 1.32-0.95 (m, 4H), 0.61-−0.05 | |
| | [1,3]oxazolidin]-3′-yl}-N- | (m, 4H). | |
| | (1,3-oxazol-4-ylmethyl)acetamide | | |
| 566 | N-benzyl-2-{(1R)-5- | ¹H NMR (400 MHz, DMSO- d₆, 120° C.) δ | (APCI⁺) |
| | [(methylcarbamoyl)amino]- | ppm 8.26 (brs, 1H), 7.48 (s, 1H), 7.45-7.31 | m/z 499 |
| | 2′,4′-dioxo-2,3-dihydro-3′H- | (m, 3H), 7.31-7.16 (m, 9H), 5.81 (brs, 1H), | (M + H)⁺ |
| | spiro[indene-1,5′- | 4.92 (s, 2H), 4.19-3.98 (m, 2H), 3.20-3.01 | |
| | [1,3]oxazolidin]-3′-yl}-N- | (m, 2H), 2.69-2.60 (m, 4H), 2.47-2.37 (m, | |
| | phenylacetamide | 1H) | |
| 567 | N-benzyl-2-{(1R)-5- | ¹H NMR (400 MHz, DMSO- d₆, 120° C.) δ | (APCI⁺) |
| | [(methylcarbamoyl)amino]- | ppm 8.25 (brs, 1H), 7.48 (s, 1H), 7.37-7.14 | m/z 527 |
| | 2′,4′-dioxo-2,3-dihydro-3′H- | (m, 12H), 6.02-5.52 (m, 2H), 4.72 (d, J = | (M + H)⁺ |
| | spiro[indene-1,5′- | 17.0 Hz, 1H), 4.59-4.23 (m, 3H), 3.20- | |
| | [1,3]oxazolidin]-3′-yl}-N- | 3.06 (m, 1H), 3.06-2.95 (m, 1H), 2.75- | |
| | [(1S)-1- | 2.59 (m, 4H), 2.49-2.40 (m, 1H), 1.50 (d, J = | |
| | phenylethyl]acetamide | 7.0 Hz, 3H) | |
| 568 | N,N-dibenzyl-2-{(1R)-5- | ¹H NMR (400 MHz, DMSO- d₆, 120° C.) δ | (APCI⁺) |
| | [(methylcarbamoyl)amino]- | ppm 8.27 (brs, 1H), 7.51-7.47 (m, 1H), | m/z 513 |
| | 2′,4′-dioxo-2,3-dihydro-3′H- | 7.36-7.20 (m, 12H), 5.84 (brs, 1H), 4.65- | (M + H)⁺ |
| | spiro[indene-1,5′- | 4.56 (m, 4H), 4.54-4.49 (m, 2H), 3.21- | |
| | [1,3]oxazoridin]-3′- | 3.07 (m, 1H), 3.07-2.96 (m, 1H), 2.73- | |
| | yl}acetamide | 2.63 (m, 4H), 2.52-2.43 (m, 1H) | |
| 572 | N-benzyl-N- | ¹H NMR (400 MHz, DMSO-d₆, | (ESI(+)) |
| | (cyclopropylmethyl)-2- | Temp = 120° C.) δ ppm 8.26 (s, 3H), 7.48 (d, J = | m/e 953 |
| | {(1R)-5- | 1.6 Hz, 3H), 7.39-7.18 (m, 21H), 4.71 (s, | (2M + H)⁺ |
| | [(methylcarbamoyl)amino]- | 6H), 4.48 (d, J = 2.8 Hz, 6H), 3.28 (d, J = 6.7 | |
| | 2′,4′-dioxo-2,3-dihydro-3′H- | Hz, 8H), 3.00 (ddd, J = 16.3, 8.7, 4.2 Hz, | |
| | spiro[indene-1,5′- | 7H), 2.66 (s, 13H), 0.99 (dddd, J = 13.3, 6.7, | |
| | [1,3]oxazoridin]-3′- | 3.2, 1.8 Hz, 3H), 0.45 (dt, J = 8.0, 3.0 Hz, | |
| | yl}acetamide | 6H), 0.21 (dt, J = 6.2, 4.4 Hz, 6H) | |
| 583 | N-(2-chlorobenzyl)-2-{(1R)- | ¹H NMR (400 MHz, DMSO-d₆) δ 8.69 (s, | (ESI(+)) |
| | 5-[(methylcarbamoyl)amino]- | 1H), 7.65-7.13 (m, 7H), 6.07 (t, J = 4.8 Hz, | m/e 513 |
| | 2′,4′-dioxo-2,3-dihydro-3′H- | 1H), 4.75 (s, 1H), 4.69-4.57 (m, 2H), 4.46 | (M + H)⁺ |
| | spiro[indene-1,5′- | (s, 1H), 3.22 (dd, J = 7.6, 2.0 Hz, 1H), 3.13 | |
| | [1,3]oxazolidin]-3′-yl}-N-(2- | (dt, J = 13.9, 7.8 Hz, 2H), 2.99 (ddt, J = 16.6, | |
| | methylpropyl)acetamide | 8.6, 4.1 Hz, 1H), 2.72-2.58 (m, 5H), 2.50 | |
| | | (pt, J = 6.4, 3.5 Hz, 1H), 2.07-1.85 (m, | |
| | | 1H), 1.00-0.78 (m, 6H) | |
| 602 | N-benzyl-N-[1-(furan-2- | ¹H NMR (400 MHz, DMSO- d₆, 120° C.) δ | (APCI⁺) |
| | yl)ethyl]-2-{(1R)-5- | ppm 8.27 (d, J = 3.3 Hz, 1H), 7.53-7.38 (m, | m/z 517 |
| | [(methylcarbamoyl)amino]- | 2H), 7.30-7.10 (m, 7H), 6.39-6.30 (m, | (M + H)⁺ |
| | 2′,4′-dioxo-2,3-dihydro-3′H- | 2H), 6.00-5.44 (m, 2H), 4.70-4.24 (m, | |
| | spiro[indene-1,5′- | 4H), 3.17-3.07 (m, 1H), 3.07-2.90 (m, | |
| | [1,3]oxazolidin]-3′- | 1H), 2.73-2.59 (m, 4H), 2.51-2.41 (m, | |
| | yl}acetamide | 1H), 1.45 (d, J = 7.0 Hz, 3H) | |
| 603 | N-benzyl-N-cyclobutyl-2- | ¹H NMR (500 MHz, DMSO-d₆) δ 8.72 (d, J = | (ESI(+)) |
| | {(1R)-5- | 6.1 Hz, 1H), 7.56 (dd, J = 7.2, 2.0 Hz, 1H), | m/e 477 |
| | [(methylcarbamoyl)amino]- | 7.40 (t, J = 7.6 Hz, 1H), 7.34-7.15 (m, 6H), | (M + H)⁺ |
| | 2′,4′-dioxo-2,3-dihydro-3′H- | 6.10 (s, 1H), 4.92-4.64 (m, 3H), 4.64-4.45 | |
| | spiro[indene-1,5′- | (m, 2H), 3.12 (dd, J = 16.3, 8.2 Hz, 1H), 2.99 | |
| | [1,3]oxazoridin]-3′- | (dtd, J = 16.3, 9.4, 3.5 Hz, 1H), 2.70-2.56 | |
| | yl}acetamide | (m, 4H), 2.55-2.39 (m, 1H), 2.20-1.89 (m, | |
| | | 4H), 1.56 (qd, J = 9.7, 5.7 Hz, 2H) | |
| 626 | N-(cyclopropylmethyl)-2- | ¹H NMR (400 MHz, DMSO-d₆) δ 8.69 (s, | (ESI(+)) |
| | {(1R)-5- | 1H), 7.56 (s, 1H), 7.49-7.13 (m, 7H), 6.08 | m/e 981 |
| | [(methylcarbamoyl)amino]- | (d, J = 4.7 Hz, 1H), 5.50 (dd, J = 160.7, 7.1 | (2M + H)⁺ |
| | 2′,4′-dioxo-2,3-dihydro-3′H- | Hz, 1H), 4.58 (d, J = 4.3 Hz, 2H), 3.30- | |
| | spiro[indene-1,5′- | 3.18 (m, 1H), 3.20-2.95 (m, 2H), 2.87 (dd, | |
| | [1,3]oxazoridin]-3′-yl}-N-(1- | J = 14.2, 6.9 Hz, 1H), 2.64 (d, J = 4.6 Hz, | |
| | phenylethyl)acetamide | 3H), 2.58-2.44 (m, 1H), 1.61 (dd, J = 37.2, | |
| | | 6.9 Hz, 3H), 1.34-1.13 (m, 1H), 0.93-0.69 | |
| | | (m, 2H), 0.59-0.03 (m, 3H), −0.14 (dq, J = | |
| | | 9.7, 4.9 Hz, 1H) | |
| 627 | 2-{(1R)-5- | ¹H NMR (400 MHz, DMSO-d₆) δ 8.69 (d, J = | (ESI(+)) |
| | [(methylcarbamoyl)amino]- | 2.1 Hz, 1H), 7.87-7.61 (m, 2H), 7.62- | m/e 1093 |
| | 2′,4′-dioxo-2,3-dihydro-3′H- | 7.45 (m, 2H), 7.46-7.11 (m, 3H), 6.07 (dd, | (2M + H)⁺ |
| | spiro[indene-1,5′- | J = 6.2, 4.6 Hz, 1H), 4.86 (s, 1H), 4.76 (d, J = | |
| | [1,3]oxazoridin]-3′-yl}-N-(2- | 7.4 Hz, 1H), 4.68 (d, J = 1.9 Hz, 1H), 4.44 | |
| | methylpropyl)-N-[2- | (s, 1H), 3.25 (dd, J = 7.5, 2.1 Hz, 1H), 3.22- | |
| | (trifluoromethyl)benzyl]acetamide | 2.93 (m, 3H), 2.64 (t, J = 4.2 Hz, 3H), 2.57- | |
| | | 2.40 (m, 1H), 2.05-1.84 (m, 1H), 1.03- | |
| | | 0.75 (m, 7H) | |
| 631 | N-benzyl-N-(cyanomethyl)- | ¹H NMR (400 MHz, DMSO- d₆, 120° C.) δ | (ESI(+)) |
| | 2-{(1R)-5- | ppm 8.26 (brs, 1H), 7.49 (brs, 1H), 7.43- | m/e 462 |
| | [(methylcarbamoyl)amino]- | 7.28 (m, 5H), 7.25 (d, J = 1.2 Hz, 2H), 5.85 | (M + H)⁺ |
| | 2′,4′-dioxo-2,3-dihydro-3′H- | (brs, 1H), 4.76 (s, 2H), 4.64-4.52 (m, 2H), | |
| | spiro[indene-1,5′- | 4.46 (s, 2H), 3.18-3.06 (m, 1H), 3.06-2.94 | |
| | [1,3]oxazolidin]-3′- | (m, 1H), 2.75-2.61 (m, 4H), 2.54-2.44 (m, | |
| | yl}acetamide | 1H) | |
| 632 | N-benzyl-N-(2-cyanoethyl)- | ¹H NMR (400 MHz, DMSO- d₆, 120° C.) δ | (ESI(+)) |
| | 2-{(1R)-5- | ppm 8.26 (brs, 1H), 7.48 (s, 1H), 7.41-7.20 | m/e 476 |
| | [(methylcarbamoyl)amino]- | (m, 7H), 5.84 (brs, 1H), 4.71 (s, 2H), 4.59- | (M + H)⁺ |
| | 2′,4′-dioxo-2,3-dihydro-3′H- | 4.48 (m, 2H), 3.64 (t, J = 6.9 Hz, 2H), 3.24- | |
| | spiro[indene-1,5′- | 3.06 (m, 1H), 3.06-2.95 (m, 1H), 2.76- | |
| | [1,3]oxazoridin]-3′- | 2.62 (m, 6H), 2.53-2.42 (m, 1H) | |
| | yl}acetamide | | |
| 642 | N-cyclopentyl-2-{(1R)-5- | ¹H NMR (400 MHz, DMSO-d₆) δ 7.51 (d, J = | (ESI(+)) |
| | [(methylcarbamoyl)amino]- | 1.9 Hz, 1H), 7.47-7.15 (m, 7H), 5.19 (q, J = | m/e 505 |
| | 2′,4′-dioxo-2,3-dihydro-3′H- | 6.8 Hz, 1H), 4.68-4.41 (m, 2H), 3.35 (t, J = | (M + H)⁺ |
| | spiro[indene-1,5′- | 8.5 Hz, 1H), 3.23-3.08 (m, 1H), 3.02 | |
| | [1,3]oxazolidin]-3′-yl}-N- | (ddd, J = 16.7, 8.9, 3.7 Hz, 1H), 2.71-2.65 | |
| | [(1S)-1- | (m, 4H), 2.59-2.46 (m, 1H), 2.04-0.99 (m, | |
| | phenylethyl]acetamide | 13H) | |
| 651 | N-benzyl-N-(2,2- | ¹H NMR (501 MHz, methanol-d₄) δ 7.51 (d, | (ESI(+)) |
| | difluorocyclopentyl)-2-{5- | J = 7.0 Hz, 1H), 7.43-7.24 (m, 5H), 7.18 | m/e 527 |
| | [(methylcarbamoyl)amino]- | (dt, J = 14.3, 6.2 Hz, 2H), 5.25 (d, J = 16.2 | (M + H)⁺ |
| | 2′,4′-dioxo-2,3-dihydro-3′H- | Hz, 1H), 5.01 (dt, J = 23.6, 11.7 Hz, 1H), | |
| | spiro[indene-1,5′- | 4.88 (d, J = 4.8 Hz, 1H), 4.66 (ddd, J = 23.3, | |
| | [1,3]oxazolidin]-3′- | 16.7, 14.4 Hz, 2H), 4.32-4.19 (m, 1H), 3.21- | |
| | yl}acetamide | 3.12 (m, 1H), 3.06 (dt, J = 16.3, 5.4 Hz, | |
| | | 1H), 2.80-2.69 (m, 4H), 2.57-2.48 (m, | |
| | | 1H), 2.17 (ddd, J = 31.2, 23.6, 11.7 Hz, 2H), | |
| | | 1.95-1.65 (m, 4H) | |
| 652 | N-(4-fluorobenzyl)-2-{5- | ¹H NMR (400 MHz, methanol-d₄) δ 7.52 (s, | (ESI(+)) |
| | [(methylcarbamoyl)amino]- | 1H), 7.39 (dd, J = 8.4, 0.9 Hz, 1H), 7.28 (dd, | m/e 495 |
| | 2′,4′-dioxo-2,3-dihydro-3′H- | J = 8.4, 5.5 Hz, 2H), 7.21 (dd, J = 8.4, 1.6 | (M + H)⁺ |
| | spiro[indene-1,5′- | Hz, 1H), 7.04 (d, J = 8.7 Hz, 2H), 4.68 (dd, J = | |
| | [1,3]oxazolidin]-3′-yl}-N-(2- | 7.4, 5.0 Hz, 1H), 4.62 (d, J = 2.9 Hz, 2H), | |
| | methylcyclopropyl)acetamide | 3.19 (dd, J = 15.8, 7.9 Hz, 1H), 3.07 (ddd, J = | |
| | | 16.3, 8.7, 3.8 Hz, 1H), 2.81-2.73 (m, 4H), | |
| | | 2.59-2.51 (m, 1H), 2.39 (dd, J = 7.0, 3.5 | |
| | | Hz, 1H), 1.34-1.23 (m, 1H), 1.07 (t, J = 5.5 | |
| | | Hz, 5H), 0.74-0.68 (m, 1H) | |
| 653 | N-(2,2- | ¹H NMR (400 MHz, methanol-d₄) δ 7.97 (s, | (ESI(+)) |
| | dimemylcyclopentyl)-N-(4- | 0.25H), 7.50 (s, 0.75H), 7.38-7.26 (m, 2H), | m/e 537 |
| | fluorobenzyl)-2-{5- | 7.16 (ddd, J = 15.8, 13.2, 7.7 Hz, 3H), 7.01- | (M + H)⁺ |
| | [(methylcarbamoyl)amino]- | 6.95 (m, 1H), 5.05 (d, J = 16.0 Hz, 0.25H), | |
| | 2′,4′-dioxo-2,3-dihydro-3′H- | 4.63 (ddd, J = 16.7, 12.9, 7.5 Hz, 1.75H), | |
| | spiro[indene-1,5′- | 4.26-3.92 (m, 1H), 3.22-3.12 (m, 1H), | |
| | [1,3]oxazolidin]-3′- | 3.07-3.02 (m, 1H), 2.99 (s, 1H), 2.86 (s, | |
| | yl}acetamide | 1H), 2.76-2.67 (m, 2H), 2.65 (s, 2H), 2.56- | |
| | | 2.46 (m, 1H), 2.08-1.96 (m, 1H), 1.92- | |
| | | 1.50 (m, 5H), 1.22 (s, 1H), 1.09 (d, J = 5.1 | |
| | | Hz, 3H), 1.00 (d, J = 5.2 Hz, 2H) | |
| 654 | N-(4-fluorobenzyl)-2-{5- | ¹H NMR (400 MHz, methanol-d₄) δ 7.52 (s, | (ESI(+)) |
| | [(methylcarbamoyl)amino]- | 1H), 7.41-7.33 (m, 2H), 7.28-7.08 (m, | m/e 523 |
| | 2′,4′-dioxo-2,3-dihydro-3′H- | 3H), 7.00 (t, J = 8.7 Hz, 1H), 4.73-4.56 (m, | (M + H)⁺ |
| | spiro[indene-1,5′- | 3H), 4.53-4.24 (m, 1H), 3.86 (t, J = 8.7 Hz, | |
| | [1,3]oxazolidin]-3′-yl}-N-(2- | 1H), 3.23-3.13 (m, 1H), 3.07 (dd, J = 12.3, | |
| | methylcyclopentyl)acetamide | 8.7 Hz, 1H), 2.82-2.69 (m, 4H), 2.60-2.45 | |
| | | (m, 1H), 2.05-1.76 (m, 3H), 1.71-1.54 (m, | |
| | | 3H), 1.32-1.19 (m, 1H), 1.01-0.86 (m, | |
| | | 3H) | |
| 662 | N-cyclobutyl-2-{(1R)-5- | ¹H NMR (400 MHz, DMSO-d₆) δ 8.72 (s, | (ESI(+)) |
| | [(methylcarbamoyl)amino]- | 1H), 7.54 (s, 1H), 7.48-7.11 (m, 7H), 6.12 | m/e 491 |
| | 2′,4′-dioxo-2,3-dihydro-3′H- | (d, J = 6.5 Hz, 1H), 5.23 (d, J = 22.9 Hz, | (M + H)⁺ |
| | spiro[indene-1,5′- | 1H), 4.65-4.33 (m, 2H), 3.79 (d, J = 15.1 | |
| | [1,3]oxazolidin]-3′-yl}-N- | Hz, 1H), 3.19-3.06 (m, 1H), 3.00 (ddd, J = | |
| | [(1S)-1- | 16.3, 9.1, 3.5 Hz, 1H), 2.64 (d, J = 3.0 Hz, | |
| | phenylethyl]acetamide | 4H), 2.50 (p, J = 1.8 Hz, 1H), 2.28-1.89 (m, | |
| | | 3H), 1.74-1.41 (m, 6H) | |
| 663 | N-cyclobutyl-2-{(1R)-5- | ¹H NMR (400 MHz, DMSO-d₆) δ 8.70 (s, | (ESI(+)) |
| | [(methylcarbamoyl)amino]- | 1H), 7.54 (d, J = 1.8 Hz, 1H), 7.33 (t, J = | m/e 491 |
| | 2′,4′-dioxo-2,3-dihydro-3′H- | 31.1 Hz, 7H), 6.09 (s, 1H), 5.21 (m, 1H), | (M + H)⁺ |
| | spiro[indene-1,5′- | 4.62-4.31 (m, 2H), 3.99 (d, J = 17.7 Hz, | |
| | [1,3]oxazolidin]-3′-yl}-N- | 1H), 3.14 (m, 1H), 2.99 (ddd, J = 16.4, 8.8, | |
| | [(1R)-1- | 3.6 Hz, 1H), 2.64 (m, 4H),), 2.55-2.40 (m, | |
| | phenylethyl]acetamide | 1H), 2.33-1.87 (m, 3H), 1.80-1.41 (m, | |
| | | 6H) | |
| 664 | N-cyclopentyl-2-{(1R)-5- | ¹H NMR (400 MHz, DMSO-d₆) δ 8.70 (s, | (ESI(+)) |
| | [(methylcarbamoyl)amino]- | 1H), 7.53 (d, J = 2.0 Hz, 1H), 7.47-7.18 (m, | m/e 505 |
| | 2′,4′-dioxo-2,3-dihydro-3′H- | 7H), 6.09 (s, 1H), 5.21 (q, J = 6.7 Hz, 1H), | (M + H)⁺ |
| | spiro[indene-1,5′- | 4.65-4.43 (m, 2H), 3.35 (p, J = 8.3 Hz, 1H), | |
| | [1,3]oxazolidin]-3′-yl}-N- | 3.19-3.06 (m, 1H), 3.01 (ddt, J = 13.1, 9.4, | |
| | [(1R)-1- | 3.9 Hz, 1H), 2.64 (m, 4H), 2.58-2.43 (m, | |
| | phenylethyl]acetamide | 1H), 1.92 (ddd, J = 20.5, 11.5, 7.5 Hz, 2H), | |
| | | 1.83-0.98 (m, 11H) | |
| 666 | N-benzyl-2-{(1R)-5- | ¹H NMR (400 MHz, DMSO- d₆, 120° C.) δ | (ESI(+)) |
| | [(methylcarbamoyl)amino]- | ppm 1.54 (d, J = 6.9 Hz, 3H), 2.41-2.46 (m, | m/e 528 |
| | 2′,4′-dioxo-2,3-dihydro-3′H- | 1H), 2.60-2.72 (m, 4H), 2.91-3.20 (m, | (M + H)⁺ |
| | spiro[indene-1,5′- | 2H), 4.35-4.66 (m, 3H), 4.77 (dd, J = 17.0, | |
| | [1,3]oxazolidin]-3′-yl}-N-[1- | 3.4 Hz, 1H), 5.48-5.69 (m, 1H), 5.79-5.87 | |
| | (pyridin-2-yl)ethyl]acetamide | (m, 1H), 7.10-7.29 (m, 8H), 7.35 (d, J = 7.9 | |
| | | Hz, 1H), 7.48 (s, 1H), 7.61-7.76 (m, 1H), | |
| | | 8.25 (s, 1H), 8.50 (d, J = 4.5 Hz, 1H) | |
| 668 | N-(4-chlorobenzyl)-2-{5- | ¹H NMR (400 MHz, DMSO-d₆) δ 8.27 (s, | (ESI(+)) |
| | [(methylcarbamoyl)amino]- | 1H), 7.48 (d, J = 1.5 Hz, 1H), 7.43-7.13 (m, | m/e 553 |
| | 2′,4′-dioxo-2,3-dihydro-3′H- | 7H), 5.29-4.20 (m, 5H), 3.16-2.90 (m, | (M + H)⁺ |
| | spiro[indene-1,5′- | 2H), 2.73-2.42 (m, 5H), 1.37 (d, J = 7.0 Hz, | |
| | [1,3]oxazolidin]-3′-yl}-N- | 3H) | |
| | [(2S)-1,1,1-trifluoropropan- | | |
| | 2-yl]acetamide | | |
| 669 | N-(3,5-difluorobenzyl)-2-{5- | ¹H NMR (400 MHz, DMSO-d₆) δ 8.25 (s, | (ESI(+)) |
| | [(methylcarbamoyl)amino]- | 1H), 7.48 (s, 1H), 7.31-6.88 (m, 6H), 5.28- | m/e 555 |
| | 2′,4′-dioxo-2,3-dihydro-3′H- | 4.18 (m, 5H), 3.18-2.91 (m, 2H), 2.74- | (M + H)⁺ |
| | spiro[indene-1,5′- | 2.39 (m, 5H), 1.39 (d, J = 7.0 Hz, 3H) | |
| | [1,3]oxazolidin]-3′-yl}-N- | | |
| | [(2S)-1,1,1-trifluoropropan- | | |
| | 2-yl]acetamide | | |
| 672 | N-cyclohexyl-2-{(1R)-5- | ¹H NMR (400 MHz, DMSO-d₆) δ 8.69 (d, J = | (ESI(+)) |
| | [(methylcarbamoyl)amino]- | 7.8 Hz, 1H), 7.53 (s, 1H), 7.49-7.14 (m, | m/e 519 |
| | 2′,4′-dioxo-2,3-dihydro-3′H- | 7H), 6.09 (s, 1H), 5.19 (q, J = 6.9 Hz, 1H), | (M + H)⁺ |
| | spiro[indene-1,5′- | 4.73-4.19 (m, 2H), 3.23-2.89 (m, 3H), | |
| | [1,3]oxazolidin]-3′-yl}-N- | 2.64 (s, 4H), 2.50 (hept, J = 3.8, 3.3 Hz, 2H), | |
| | [(1R)-1-phenylethyl]acetamide | 2.24-0.73 (m, 13H) | |
| 708 | 2-{(1R)-5- | ¹H NMR (400 MHz, DMSO-d₆) δ 8.70 (s, | (ESI(+)) |
| | [(methylcarbamoyl)amino]- | 1H), 7.63-7.53 (m, 1H), 7.53-7.41 (m, | m/e 1013 |
| | 2′,4′-dioxo-2,3-dihydro-3′H- | 1H), 7.40-7.10 (m, 5H), 6.08 (d, J = 4.8 Hz, | (2M + H)⁺ |
| | spiro[indene-1,5′- | 1H), 5.87-5.20 (m, 1H), 4.69-4.56 (m, | |
| | [1,3]oxazolidin]-3′-yl}-N-[1- | 1H), 4.54-4.39 (m, 1H), 3.22-2.87 (m, | |
| | (2-methylphenyl)ethyl]-N- | 3H), 2.65 (dd, J = 4.6, 1.8 Hz, 3H), 2.58- | |
| | (2-methylpropyl)acetamide | 2.46 (m, 1H), 2.38 (d, J = 2.3 Hz, 1H), 2.14 | |
| | | (d, J = 10.9 Hz, 2H), 1.60 (d, J = 6.8 Hz, | |
| | | 1H), 1.53-1.42 (m, 2H), 1.35-1.21 (m, | |
| | | 1H), 0.84 (q, J = 8.1, 7.2 Hz, 1H), 0.66 (ddd, | |
| | | J = 23.5, 6.7, 2.4 Hz, 6H), 0.44 (t, J = 6.2 | |
| | | Hz, 1H) | |
| 709 | N-(2-chlorobenzyl)-N- | ¹H NMR (400 MHz, DMSO-d₆) δ 8.69 (d, J = | (ESI(+)) |
| | (cyclopropylmethyl)-2- | 3.2 Hz, 1H), 7.66-6.96 (m, 7H), 6.07 (t, J = | m/e 511 |
| | {(1R)-5- | 4.8 Hz, 1H), 4.84 (s, 1H), 4.71 (dd, J = | (M + H)⁺ |
| | [(methylcarbamoyl)amino]- | 19.5, 5.2 Hz, 2H), 4.46 (d, J = 3.1 Hz, 1H), | |
| | 2′,4′-dioxo-2,3-dihydro-3′H- | 3.36 (d, J = 6.8 Hz, 1H), 3.28-2.94 (m, 3H), | |
| | spiro[indene-1,5′- | 2.64 (dd, J = 4.6, 3.3 Hz, 4H), 2.55-2.42 | |
| | [1,3]oxazolidin]-3′- | (m, 2H), 1.11-0.78 (m, 2H), 0.59-0.34 (m, | |
| | yl}acetamide | 2H), 0.31-0.08 (m, 2H) | |
| 710 | N-(2-fluorobenzyl)-2-{(1R)- | ¹H NMR (501 MHz, DMSO-d₆) δ 8.69 (s, | (ESI(+)) |
| | 5-[(methylcarbamoyl)amino]- | 1H), 7.55 (dd, J = 3.7, 1.9 Hz, 1H), 7.48- | m/e 993 |
| | 2′,4′-dioxo-2,3-dihydro-3′H- | 7.04 (m, 6H), 6.17-6.00 (m, 1H), 4.78- | (2M + H)⁺ |
| | spiro[indene-1,5′- | 4.69 (m, 1H), 4.68-4.47 (m, 3H), 3.24- | |
| | [1,3]oxazolidin]-3′-yl}-N-(2- | 3.06 (m, 3H), 3.00 (ddt, J = 16.7, 8.7, 4.3 Hz, | |
| | methylpropyl)acetamide | 1H), 2.64 (dd, J = 4.6, 1.9 Hz, 4H), 2.56- | |
| | | 2.42 (m, 2H), 2.11-1.89 (m, 1H), 0.95- | |
| | | 0.81 (m, 6H) | |
| 720 | N-(3,4-difluorobenzyl)-2-{5- | ¹H NMR (400 MHz, DMSO-d₆) δ 8.69 (d, J = | (ESI(+)) |
| | [(methylcarbamoyl)amino]- | 3.8 Hz, 1H), 7.63-6.97 (m, 6H), 6.07 (s, | m/e 577 |
| | 2′,4′-dioxo-2,3-dihydro-3′H- | 1H), 5.52-4.14 (m, 5H), 3.20-2.88 (m, | (M + H)⁺ |
| | spiro[indene-1,5′- | 2H), 2.74-2.40 (m, 5H), 1.44-1.30 (m, 3H) | |
| | [1,3]oxazolidin]-3′-yl}-N- | | |
| | [(2S)-1,1,1-trifluoropropan- | | |
| | 2-yl]acetamide | | |
| 721 | N-(3-fluorobenzyl)-2-{5- | ¹H NMR (501 MHz, DMSO-d₆) δ 8.70 (dd, J = | (ESI(+)) |
| | [(methylcarbamoyl)amino]- | 6.1, 2.2 Hz, 1H), 7.61-6.92 (m, 7H), 6.08 | m/e 559 |
| | 2′,4′-dioxo-2,3-dihydro-3′H- | (s, 1H), 5.52-4.12 (m, 5H), 3.21-2.91 (m, | (M + H)⁺ |
| | spiro[indene-1,5′- | 2H), 2.75-2.39 (m, 5H), 1.42-1.31 (m, 3H) | |
| | [1,3]oxazolidin]-3′-yl}-N- | | |
| | [(2S)-1,1,1-trifluoropropan- | | |
| | 2-yl]acetamide | | |
| 722 | N-(2,5-difluorobenzyl)-2-{5- | ¹H NMR (501 MHz, DMSO-d₆) δ 8.78- | (ESI(+)) |
| | [(methylcarbamoyl)amino]- | 8.61 (m, 1H), 7.55 (d, J = 4.0 Hz, 1H), 7.42- | m/e 577 |
| | 2′,4′-dioxo-2,3-dihydro-3′H- | 6.85 (m, 5H), 6.07 (d, J = 6.8 Hz, 1H), 5.49- | (M + H)⁺ |
| | spiro[indene-1,5′- | 4.24 (m, 5H), 3.24-2.90 (m, 2H), 2.70- | |
| | [1,3]oxazolidin]-3′-yl}-N- | 2.40 (m, 5H), 1.49-1.30 (m, 3H) | |
| | [(2S)-1,1,1-trifluoropropan- | | |
| | 2-yl]acetamide | | |
| 723 | N-(3-chlorobenzyl)-2-{5- | ¹H NMR (501 MHz, DMSO-d₆) δ 8.69 (dd, J = | (ESI(+)) |
| | [(methylcarbamoyl)amino]- | 5.5, 2.1 Hz, 1H), 7.59-7.52 (m, 1H), 7.48- | m/e 575 |
| | 2′,4′-dioxo-2,3-dihydro-3′H- | 7.11 (m, 6H), 6.07 (s, 1H), 5.50-4.12 (m, | (M + H)⁺ |
| | spiro[indene-1,5′- | 5H), 3.22-2.93 (m, 2H), 2.78-2.36 (m, | |
| | [1,3]oxazolidin]-3′-yl}-N- | 5H), 1.44-1.30 (m, 3H) | |
| | [(2S)-1,1,1-trifluoropropan- | | |
| | 2-yl]acetamide | | |
| 733 | N-(cyclopropylmethyl)-2- | ¹H NMR (400 MHz, DMSO-d₆) δ 8.66 (d, J = | (ESI(+)) |
| | {(1R)-5- | 4.5 Hz, 1H), 7.83-7.38 (m, 1H), 7.36- | m/e 567 |
| | [(methylcarbamoyl)amino]- | 7.10 (m, 6H), 6.03 (dd, J = 6.3, 4.6 Hz, 1H), | (M + H)⁺ |
| | 2′,4′-dioxo-2,3-dihydro-3′H- | 4.99-4.54 (m, 3H), 4.40 (d, J = 3.7 Hz, 1H), | |
| | spiro[indene-1,5′- | 3.45-3.18 (m, 2H), 3.19-2.85 (m, 2H), | |
| | [1,3]oxazolidin]-3′-yl}-N-[2- | 2.73-2.56 (m, 4H), 2.56-2.35 (m, 1H), | |
| | (trifluoromethyl)benzyl]acetamide | 1.06-0.64 (m, 2H), 0.55-0.28 (m, 2H), | |
| | | 0.15 (ddd, J = 28.5, 4.9, 1.5 Hz, 2H) | |
| 734 | N-(2-chloro-4- | ¹H NMR (400 MHz, DMSO-d₆) δ 8.65 (d, J = | (ESI(+)) |
| | fluorobenzyl)-N- | 2.2 Hz, 1H), 7.59-7.05 (m, 6H), 6.03 (t, J = | m/e 551 |
| | (cyclopropylmethyl)-2- | 4.5 Hz, 1H), 4.81-4.34 (m, 4H), 3.29 (s, | (M + H)⁺ |
| | {(1R)-5-[(methylcarbamoyl)amino]- | 2H), 3.17-2.88 (m, 2H), 2.74-2.52 (m, | |
| | 2′,4′-dioxo-2,3-dihydro-3′H- | 3H), 2.55-2.36 (m, 1H), 1.07-0.68 (m, | |
| | spiro[indene-1,5′- | 2H), 0.55-0.29 (m, 2H), 0.16 (ddd, J = | |
| | [1,3]oxazolidin]-3′- | 48.2, 5.0, 1.6 Hz, 2H) | |
| | yl}acetamide | | |
| 737 | N-benzyl-2-{(1R)-5- | ¹H NMR (400 MHz, DMSO-d₆, 120° C.) δ | (ESI(+)) |
| | [(methylcarbamoyl)amino]- | ppm 8.64-8.54 (m, 2H), 8.28 (brs, 1H), | m/e 500 |
| | 2′,4′-dioxo-2,3-dihydro-3′H- | 7.48 (d, J = 1.7 Hz, 1H), 7.40-7.34 (m, 2H), | (M + H)⁺ |
| | spiro[indene-1,5′- | 7.33-7.16 (m, 7H), 5.85 (brs, 1H), 5.03 (s, | |
| | [1,3]oxazolidin]-3′-yl}-N- | 2H), 4.42-4.24 (m, 2H), 3.19-2.92 (m, | |
| | (pyridin-4-yl)acetamide | 3H), 2.74-2.58 (m, 4H) | |
| 758 | N-cyclohexyl-2-{(1R)-5- | ¹H NMR (400 MHz, DMSO-d₆) δ 8.69 (d, J = | (ESI(+)) |
| | [(methylcarbamoyl)amino]- | 6.3 Hz, 1H), 7.53 (s, 1H), 7.42 (dq, J = | m/e 519 |
| | 2′,4′-dioxo-2,3-dihydro-3′H- | 13.3, 6.6, 5.4 Hz, 2H), 7.29 (tt, J = 18.5, 7.5 | (M + H)⁺ |
| | spiro[indene-1,5′- | Hz, 3H), 7.15 (t, J = 8.2 Hz, 2H), 6.09 (s, | |
| | [1,3]oxazolidin]-3′-yl}-N- | 1H), 4.68-4.20 (m, 2H), 3.96 (m, 2H), 3.20- | |
| | [(1S)-1- | 2.91 (m, 3H), 2.64 (m, 4H), 2.55-2.40 (m, | |
| | phenylethyl]acetamide | 1H), 2.26-1.89 (m, 1H), 1.89-1.69 (m, | |
| | | 2H), 1.65 (d, J = 6.7 Hz, 3H), 1.60-1.41 (m, | |
| | | 2H), 1.40-0.72 (m, 4H). | |
| 778 | tert-butyl 4-[(4- | ¹H NMR (400 MHz, CD₃OD) δ 7.52 (s, 1H), | (ESI(+)) |
| | fluorobenzyl)({(1R)-5- | 7.38 (td, J = 8.3, 4.9 Hz, 2H), 7.27-7.18 (m, | m/e 645 |
| | [(methylcarbamoyl)amino]- | 2H), 7.12 (td, J = 8.8, 3.2 Hz, 1H), 7.00 (td, J = | (M + H)⁺ |
| | 2′,4′-dioxo-2,3-dihydro-3′H- | 8.8, 2.2 Hz, 1H), 4.71-4.57 (m, 3H), 4.43 | |
| | spiro[indene-1,5′- | (dd, J = 15.0, 5.1 Hz, 1H), 4.30 (d, J = 9.4 | |
| | [1,3]oxazolidin]-3′- | Hz, 1H), 3.25-3.14 (m, 1H), 3.06 (ddd, J = | |
| | yl}acetyl)amino]cyclo- | 16.5, 8.9, 4.4 Hz, 1H), 2.82-2.67 (m, 4H), | |
| | hexanecarboxylate | 2.13 (s, 2H), 1.97 (t, J = 12.1 Hz, 2H), 1.84- | |
| | | 1.68 (m, 1H), 1.67-1.46 (m, 5H), 1.44- | |
| | | 1.37 (m, 9H) | |
| 811 | 4-[(4-fluorobenzyl)({(1R)-5- | ¹H NMR (400 MHz, DMSO-d₆, 120° C.) δ | (ESI(+)) |
| | [(methylcarbamoyl)amino]- | ppm 8.41 (brs, 1H), 7.90 (d, J = 8.2 Hz, 2H), | m/e 560 |
| | 2′,4′-dioxo-2,3-dihydro-3′H- | 7.52-7.28 (m, 4H), 7.28-7.19 (m, 4H), | (M + H)⁺ |
| | spiro[indene-1,5′- | 7.12-7.02 (m, 2H), 5.93 (brs, 1H), 4.95 (s, | |
| | [1,3]oxazoridin]-3′- | 2H), 4.21-4.06 (m, 2H), 3.17-2.93 (m, | |
| | yl}acetyl)amino]benzamide | 2H), 2.70-2.56 (m, 4H), 2.48-2.40 (m, | |
| | | 1H) | |
| 812 | tert-butyl 2-{[(4- | ¹H NMR (400 MHz, DMSO-d₆, 120° C.) δ | (ESI(+)) |
| | fluorobenzyl)({(1R)-5- | ppm 8.26 (brs, 1H), 7.48 (s, 1H), 7.35-7.20 | m/e 646 |
| | [(methylcarbamoyl)amino]- | (m, 4H), 7.12 (t, J = 8.7 Hz, 2H), 5.89-5.80 | (M + Na)⁺ |
| | 2′,4′-dioxo-2,3-dihydro-3′H- | (m, 1H), 4.72 (dd, J = 16.4, 5.7 Hz, 1H), 4.66- | |
| | spiro[indene-1,5′- | 4.36 (m, 3H), 4.09-3.98 (m, 1H), 3.52- | |
| | [1,3]oxazolidin]-3′- | 3.18 (m, 4H), 3.17-2.96 (m, 2H), 2.75- | |
| | yl}acetyl)amino]methyl}pyr- | 2.60 (m, 4H), 2.51-2.40 (m, 1H), 1.94- | |
| | rolidine-1-carboxylate | 1.69 (m, 4H), 1.38 (s, 9H) | |
| 815 | N-ethyl-4-[(4- | ¹H NMR (400 MHz, CD₃OD) δ 7.52 (s, 1H), | (ESI(+)) |
| | fluorobenzyl)({(1R)-5- | 7.39 (ddd, J = 12.7, 6.7, 3.0 Hz, 2H), 7.30- | m/e 594 |
| | [(methylcarbamoyl)amino]- | 7.07 (m, 3H), 6.99 (td, J = 8.8, 6.6 Hz, 1H), | (M + H)⁺ |
| | 2′,4′-dioxo-2,3-dihydro-3′H- | 4.73-4.57 (m, 3H), 4.50-4.23 (m, 1H), | |
| | spiro[indene-1,5′- | 3.92-3.81 (m, 1H), 3.22-3.01 (m, 4H), | |
| | [1,3]oxazolidin]-3′- | 2.82-2.69 (m, 4H), 2.62-2.39 (m, 2H), | |
| | yl}acetyl)amino]cyclo- | 2.11-1.44 (m, 9H), 1.15-1.02 (m, 3H) | |
| | hexanecarboxamide | | |
| 825 | 3-[(4-fluorobenzyl)({(1R)-5- | ¹H NMR (400 MHz, DMSO-d₆, 120° C.) δ | (ESI(+)) |
| | [(methylcarbamoyl)amino]- | ppm 8.27 (brs, 1H), 7.86 (d, J = 7.7 Hz, 1H), | m/e 560 |
| | 2′,4′-dioxo-2,3-dihydro-3′H- | 7.81-7.75 (m, 1H), 7.53-7.44 (m, 2H), | (M + H)⁺ |
| | spiro[indene-1,5′- | 7.38-7.19 (m, 7H), 7.04 (t, J = 8.7 Hz, 2H), | |
| | [1,3]oxazolidin]-3′- | 5.90-5.79 (m, 1H), 4.94 (s, 2H), 4.13 (d, J = | |
| | yl}acetyl)amino]benzamide | 3.9 Hz, 2H), 3.18-2.91 (m, 2H), 2.71-2.59 | |
| | | (m, 4H), 2.47-2.39 (m, 1H) | |
| 831 | N-(cyclopropylmethyl)-N- | ¹H NMR (400 MHz, DMSO-d₆) ppm 8.76 | (ESI(+)) |
| | (2,3-dihydro-1H-inden-1- | (s, 1H), 7.70-7.59 (m, 1H), 7.47-7.12 (m, | m/e 503 |
| | yl)-2-{(1R)-5- | 6H), 6.15 (p, J = 4.7 Hz, 1H), 5.95-5.63 (m, | (M + H)⁺ |
| | [(methylcarbamoyl)amino]- | 1H), 4.88-4.54 (m, 2H), 3.39-2.84 (m, | |
| | 2′,4′-dioxo-2,3-dihydro-3′H- | 7H), 2.82-2.58 (m, 6H), 2.52-2.12 (m, | |
| | spiro[indene-1,5′- | 2H), 0.98-0.85 (m, 1H), 0.59-0.31 (m, | |
| | [1,3]oxazolidin]-3′- | 2H), 0.27-−0.09 (m, 2H) | |
| | yl}acetamide | | |
| 849 | N-(3-cyanophenyl)-N-(4- | ¹H NMR (400 MHz, DMSO-d₆, 120° C.) δ | (ESI(+)) |
| | fluorobenzyl)-2-{(1R)-5- | ppm 8.31-8.22 (m, 1H), 7.81-7.72 (m, | m/e 542 |
| | [(methylcarbamoyl)amino]- | 2H), 7.65-7.51 (m, 2H), 7.48 (brs, 1H), | (M + H)⁺ |
| | 2′,4′-dioxo-2,3-dihydro-3′H- | 7.32-7.17 (m, 4H), 7.05 (t, J = 8.7 Hz, 2H), | |
| | spiro[indene-1,5′- | 5.90-5.77 (m, 1H), 4.95 (s, 2H), 4.28-4.11 | |
| | [1,3]oxazolidin]-3′- | (m, 2H), 3.25-2.92 (m, 2H), 2.73-2.60 (m, | |
| | yl}acetamide | 4H), 2.48-2.40 (m, 1H) | |
| 878 | benzyl 3-{[(4- | ¹H NMR (400 MHz, DMSO-d₆, 120° C.) δ | (ESI(+)) |
| | fluorobenzyl)({(1R)-5- | ppm 8.26 (brs, 1H), 7.51-7.44 (m, 1H), | m/e 644 |
| | [(methylcarbamoyl)amino]- | 7.38-7.17 (m, 9H), 7.12 (t, J = 8.7 Hz, 2H), | (M + H)⁺ |
| | 2′,4′-dioxo-2,3-dihydro-3′H- | 5.90-5.78 (m, 1H), 5.00 (s, 2H), 4.61 (brs, | |
| | spiro[indene-1,5′- | 2H), 4.51-4.42 (m, 2H), 3.88 (t, J = 8.4 Hz, | |
| | [1,3]oxazoridin]-3′- | 2H), 3.69-3.56 (m, 4H), 3.16-3.06 (m, | |
| | yl}acetyl)amino]methyl}azetidine- | 1H), 3.04-2.94 (m, 1H), 2.92-2.85 (m, | |
| | 1-carboxylate | 1H), 2.72-2.61 (m, 4H), 2.51-2.40 (m, | |
| | | 1H) | |
| 879 | tert-butyl 4-[(4- | ¹H NMR (400 MHz, DMSO-d₆, 120° C.) δ | (ESI(+)) |
| | fluorobenzyl)({(1R)-5- | ppm 8.26 (brs, 1H), 7.96-7.88 (m, 2H), 7.49- | m/e 634 |
| | [(methylcarbamoyl)amino]- | 7.45 (m, 1H), 7.40-7.34 (m, 2H), 7.28- | (M + NH4)⁺ |
| | 2′,4′-dioxo-2,3-dihydro-3′H- | 7.18 (m, 4H), 7.09-6.99 (m, 2H), 5.90- | |
| | spiro[indene-1,5′- | 5.80 (m, 1H), 4.94 (brs, 2H), 4.19-4.11 (m, | |
| | [1,3]oxazolidin]-3′- | 2H), 3.16-3.06 (m, 1H), 3.03-2.92 (m, | |
| | yl}acetyl)amino]benzoate | 1H), 2.70-2.59 (m, 4H), 2.45-2.38 (m, | |
| | | 1H), 1.54 (s, 9H) | |
| 895 | tert-butyl 3-[(4- | ¹H NMR (400 MHz, DMSO-d₆, 120° C.) δ | (ESI(+)) |
| | fluorobenzyl)({(1R)-5- | ppm 8.32-8.21 (m, 1H), 7.90-7.83 (m, | m/e 1233 |
| | [(methylcarbamoyl)amino]- | 1H), 7.69-7.63 (m, 1H), 7.56-7.45 (m, | (2M + H)⁺ |
| | 2′,4′-dioxo-2,3-dihydro-3′H- | 3H), 7.29-7.19 (m, 4H), 7.09-7.00 (m, | |
| | spiro[indene-1,5′- | 2H), 5.91-5.79 (m, 1H), 4.92 (s, 2H), 4.18- | |
| | [1,3]oxazolidin]-3′- | 4.06 (m, 2H), 3.18-3.05 (m, 1H), 3.04- | |
| | yl}acetyl)amino]benzoate | 2.93 (m, 1H), 2.70-2.58 (m, 4H), 2.46- | |
| | | 2.38 (m, 1H), 1.52 (s, 9H) | |
| 897 | tert-butyl {4-[(4- | ¹H NMR (400 MHz, DMSO-d₆, 120° C.) δ | (ESI(+)) |
| | fluorobenzyl)({(1R)-5- | ppm 8.25 (brs, 1H), 7.53-7.42 (m, 1H), | m/e 1261 |
| | [(methylcarbamoyl)amino]- | 7.34-7.26 (m, 2H), 7.26-7.13 (m, 6H), | (2M + H)⁺ |
| | 2′,4′-dioxo-2,3-dihydro-3′H- | 7.08-6.98 (m, 2H), 5.91-5.77 (m, 1H), | |
| | spiro[indene-1,5′- | 4.88 (s, 2H), 4.16-4.01 (m, 2H), 3.54 (s, | |
| | [1,3]oxazolidin]-3′- | 2H), 3.18-3.04 (m, 1H), 3.04-2.93 (m, | |
| | yl}acetyl)amino]phenyl}acetate | 1H), 2.70-2.58 (m, 4H), 2.46-2.39 (m, | |
| | | 1H), 1.38 (s, 9H) | |
| 901 | methyl {3-[(4- | ¹H NMR (400 MHz, DMSO-d₆, 120° C.) δ | (ESI(+)) |
| | fluorobenzyl)({(1R)-5- | ppm 8.26 (brs, 1H), 7.52-7.44 (m, 1H), | m/e 1177 |
| | [(methylcarbamoyl)amino]- | 7.37 (t, J = 7.7 Hz, 1H), 7.31-7.09 (m, 7H), | (2M + H)⁺ |
| | 2′,4′-dioxo-2,3-dihydro-3′H- | 7.08-6.98 (m, 2H), 5.93-5.77 (m, 1H), | |
| | spiro[indene-1,5′- | 4.88 (s, 2H), 4.15-4.04 (m, 2H), 3.65 (s, | |
| | [1,3]oxazolidin]-3′- | 2H), 3.60 (s, 3H), 3.16-3.06 (m, 1H), 3.04- | |
| | yl}acetyl)amino]phenyl}acetate | 2.92 (m, 1H), 2.70-2.58 (m, 4H), 2.46- | |
| | | 2.38 (m, 1H) | |

### Example 379

### N-(4-fluorobenzyl)-2-(3′-fluoro-2,5-dioxo-2′,3′-dihydro-1H-spiro[imidazolidine-4,1′-inden]-1-yl)-N-[(2S)-1,1,1-trifluoropropan-2-yl]acetamide

N-(4-fluorobenzyl)-2-(3′-hydroxy-2,5-dioxo-2′,3′-dihydro-1H-spiro[imidazolidine-4,1′-inden]-1-yl)-N-[(2S)-1,1,1-trifluoropropan-2-yl]acetamide (95 mg, 0.198 mmol) in 2 mL dichloromethane was cooled to −78° C. Diethylaminosulfur trifluoride (105 μl, 0.793 mmol) was added via syringe and the mixture was stirred at −78° C. for 30 minutes. The cooling bath was removed and stirring was continued with the internal temperature changing from −78° C. to 0° C. The solution was concentrated to dryness and the residue was purified by column chromatography to give the title compound. ¹H NMR (400 MHz, DMSO-d₆) δ 9.16-8.62 (m, 1H), 7.71-6.95 (m, 8H), 6.41-5.95 (m, 1H), 5.21 (ddt, J=113.4, 14.2, 7.5 Hz, 1H), 4.96-3.99 (m, 4H), 3.05 (dddd, J=18.7, 11.9, 6.7, 2.9 Hz, 1H), 2.34 (dddd, J=22.9, 14.7, 10.2, 4.3 Hz, 1H), 1.64-1.20 (m, 3H). MS (ESI⁺) m/z 504 (M+Na)⁺.

### Example 380

### 2-(3′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

N-[(1S)-1-cyclopropylethyl]-N-(4-fluorobenzyl)-2-(2,3′,5-trioxo-2′,3′-dihydro-1H-spiro[imidazolidine-4,1′-inden]-1-yl)acetamide (290 mg, 0.607 mmol) in 7 mL pH 4 buffer was treated with ammonium acetate (468 mg, 6.07 mmol). The mixture was stirred at room temperature for 30 minutes and then sodium cyanotrihydroborate (115 mg, 1.822 mmol) was added. The mixture was stirred at room temperature for 3 hours and at 70° C. for 4 hours. The mixture was diluted with water and extracted with ethyl acetate. Silica gel column chromatography gave the title compound. ¹H NMR (400 MHz, DMSO-d₆) δ 8.96 (d, J=10.0 Hz, 1H), 7.45 (m, 8H), 5.34 (m, 1H), 5.10-4.15 (m, 7H), 2.96-2.0 (m, 2H), 1.44 (dq, J=20.4, 6.4 Hz, 3H). MS (ESI⁺) m/z 479 (M+H)⁺.

### Example 381

### N-(4-fluorobenzyl)-2-[3′-(methylamino)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 380, substituting methylamine hydrochloride for ammonium acetate. ¹H NMR (400 MHz, DMSO-d₆) δ 8.82 (s, 1H), 7.47-7.09 (m, 8H), 5.59-3.71 (m, 6H), 2.80-2.67 (m, 1H), 2.46-2.37 (m, 1H), 2.36-2.30 (m, 3H), 2.1-2.0 (m, 1H), 1.32 (m, 3H). MS (ESI⁺) m/z 493 (M+H)⁺.

### Example 382

### N-[(1S)-1-cyclopropyl-2,2,2-trifluoroethyl]-N-(4-fluorobenzyl)-2-{7′-[(methylcarbamoyl)amino]-2,4-dioxo-2′,3′-dihydro-3H-spiro[1,3-oxazolidine-54′-thiochromen]-3-yl}acetamide

### Example 382A

### 7′-bromospiro[oxazolidine-5,4′-thiochroman]-2,4-dione

The title compound was prepared as described in EXAMPLE 301A-D, substituting 7-bromothiochroman-4-one for 7-bromochroman-4-one in EXAMPLE 301A.

### Example 382B

### 2-(7′-bromo-2,4-dioxospiro[oxazolidine-5,4′-thiochroman]-3-yl)-N—((S)-1-cyclopropyl-2,2,2-trifluoroethyl)-N-(4-fluorobenzyl)acetamide

The title compound was prepared as described in EXAMPLE 281F, substituting 7′-bromospiro[oxazolidine-5,4′-thiochroman]-2,4-dione for (R)-5-bromo-2,3-dihydrospiro[indene-1,5′-oxazolidine]-2′,4′-dione and (S)-2-bromo-N-(1-cyclopropyl-2,2,2-trifluoroethyl)-N-(4-fluorobenzyl)acetamide for (S)-2-bromo-N-(4-fluorobenzyl)-N-(1,1,1-trifluoropropan-2-yl)acetamide.

### Example 382C

### 2-(7′-amino-2,4-dioxospiro[oxazolidine-5,4′-thiochroman]-3-yl)-N—((S)-1-cyclopropyl-2,2,2-trifluoroethyl)-N-(4-fluorobenzyl)acetamide

The title compound was prepared as described in EXAMPLE 284D-E, substituting 2-(7′-bromo-2,4-dioxospiro[oxazolidine-5,4′-thiochroman]-3-yl)-N—((S)-1-cyclopropyl-2,2,2-trifluoroethyl)-N-(4-fluorobenzyl)acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide.

### Example 382D

### N-[(1S)-1-cyclopropyl-2,2,2-trifluoroethyl]-N-(4-fluorobenzyl)-2-{7′-[(methylcarbamoyl)amino]-2,4-dioxo-2′,3′-dihydro-3H-spiro[,3-oxazolidine-5,4′-thiochromen]-3-yl}acetamide

The title compound was prepared as described in EXAMPLE 282, substituting 2-(7′-amino-2,4-dioxospiro[oxazolidine-5,4′-thiochroman]-3-yl)-N—((S)-1-cyclopropyl-2,2,2-trifluoroethyl)-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, dimethylsulfoxide-d₆) δ ppm 0.08-0.42 (m, 2H), 0.63-0.86 (m, 2H), 1.13-1.34 (m, 1H), 2.19-2.39 (m, 1H), 2.52-2.69 (m, 4H), 3.00-3.22 (m, 2H), 4.03-4.66 (m, 3H), 4.79-5.12 (m, 2H), 6.12 (s, 1H), 6.93-7.52 (m, 7H), 8.69 (s, 1H).). MS (ESI⁺) m/z 595 (M+H)⁺.

### Example 383

### 2-[(1R)-5-(carbamoylamino)-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 282, substituting 2-((R)-5-amino-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and ammonium acetate for 3,3-difluoroazetidine hydrochloride. ¹H NMR (400 MHz, DMSO-d₆) δ 8.71 (s, 1H), 7.59 (d, J=5.6 Hz, 1H), 7.38 (dd, J=8.4, 5.4 Hz, 1H), 7.34-7.09 (m, 5H), 5.92 (s, 2H), 5.50-5.30 (m, 0.7H), 5.06 (d, J=7.3 Hz, 0.3H), 4.88 (ddd, J=59.3, 29.9, 16.2 Hz, 2H), 4.63-4.38 (m, 1.3H), 4.21 (d, J=17.0 Hz, 0.7H), 3.12 (dt, J=15.0, 6.9 Hz, 1H), 2.99 (dd, J=12.4, 8.9 Hz, 1H), 2.72-2.57 (m, 1H), 2.57-2.42 (m, 1H), 1.36 (dd, J=17.8, 6.9 Hz, 3H). MS (ESI⁺) m/z 545 (M+Na)⁺.

### Example 384

### 2-(7′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide

The title compound was prepared as described in EXAMPLE 296A-B, substituting 2-(7′-bromo-2,5-dioxo-3′,4′-dihydro-1H, 1′H-spiro[imidazolidine-4,2′-naphthalen]-1-yl)-N-[(1S)-1-cyclopropylethyl]-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide. ¹H NMR (400 MHz, DMSO-d₆, 90° C.) δ ppm 8.20 (brs, 1H), 7.42-7.25 (m, 2H), 7.17-6.98 (m, 2H), 6.78 (d, J=8.1 Hz, 1H), 6.41 (dd, J=8.0, 2.3 Hz, 1H), 6.33-6.28 (m, 1H), 4.72-4.44 (m, 4H), 4.39-4.09 (m, 2H), 3.71-3.22 (m, 1H), 3.04 (d, J=17.0 Hz, 1H), 2.80-2.68 (m, 2H), 2.62 (d, J=16.8 Hz, 1H), 2.01-1.90 (m, 1H), 1.85-1.74 (m, 1H), 1.17-1.10 (m, 3H), 1.01-0.88 (m, 1H), 0.54-0.35 (m, 1H), 0.32-0.13 (m, 3H). MS (ESI⁺) m/z 487 (M+Na)⁺.

### Example 385

### N-[(1S)-1-cyclopropyl-2,2,2-trifluoroethyl]-N-(4-fluorobenzyl)-2-{7′-[(methylcarbamoyl)amino]-1′,1′-dioxido-2,4-dioxo-2′,3′-dihydro-3H-spiro[1,3-oxazolidine-5,4′-thiochromen]-3-yl}acetamide

To a mixture of N-[(1S)-1-cyclopropyl-2,2,2-trifluoroethyl]-N-(4-fluorobenzyl)-2-{7′-[(methylcarbamoyl)amino]-2,4-dioxo-2′,3′-dihydro-3H-spiro[1,3-oxazolidine-5,4′-thiochromen]-3-yl}acetamide (10 mg, 0.017 mmol) in dichloromethane (168 μl) was added m-chloroperoxybenzoic acid (14.51 mg, 0.084 mmol). The reaction mixture was stirred at room temperature for 1 hour. Concentration and reverse-phase prep HPLC provided the title compound. ¹H NMR (400 MHz, dimethylsulfoxide-d₆) δ ppm 0.07-0.89 (m, 4H), 1.16-1.38 (m, 1H), 2.56-3.06 (m, 4H), 3.58-5.16 (m, 6H), 6.22-6.39 (m, 1H), 7.04-7.33 (m, 2H), 7.37-7.64 (m, 3H), 8.12-8.27 (m, 1H), 9.14-9.32 (m, 1H). MS (ESI⁺) m/z 649 (M+H)⁺.

### Example 386

### 2-(6-bromo-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide

The title compound was prepared as described in EXAMPLE 281F, substituting 6-bromo-2H-spiro[benzo[b]thiophene-3,4′-imidazolidine]-2′,5′-dione 1,1-dioxide for (R)-5-bromo-2,3-dihydrospiro[indene-1,5′-oxazolidine]-2′,4′-dione. ¹H NMR (400 MHz, DMSO-d₆, 90° C.) δ ppm 9.01 (brs, 1H), 8.09 (d, J=1.9 Hz, 1H), 8.05-7.97 (m, 1H), 7.63 (dd, J=8.4, 6.8 Hz, 1H), 7.41-7.23 (m, 2H), 7.22-7.06 (m, 2H), 5.20 (brs, 1H), 4.83 (dd, J=17.6, 8.4 Hz, 1H), 4.75-4.35 (m, 2H), 4.38-4.15 (m, 1H), 4.03 (dd, J=14.5, 5.9 Hz, 1H), 3.85 (dd, J=14.6, 5.1 Hz, 1H), 1.41-1.30 (m, 3H). MS (ESI⁺) m/z 614 (M+Na)⁺.

### Example 387

### N-(4-fluorobenzyl)-2-((R)-5-(3-(N-methylsulfamoyl)ureido)-2′,4′-dioxo-2,3-dihydrospiro[indene-1,5′-oxazolidine]-3′-yl)-N—((S)-1,1,1-trifluoropropan-2-yl)acetamide

To a solution of 2-((R)-5-amino-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide (40 mg, 0.083 mmol) in 1 ml tetrahydrofuran at −40° C. was added sulfurisocyanatidic chloride (100 μl, 0.100 mmol, 1.0 M tetrahydrofuran solution) and the solution turned yellow after addition. The mixture was stirred at −40° C. to −30° C. for 30 minutes, and then methanamine (167 μl, 0.334 mmol, 2.0 M tetrahydrofuran solution) was added. The mixture was stirred at room temperature for 1 hour and quenched with water. The aqueous layer was extracted with ethyl acetate, washed with brine, dried over MgSO₄, filtered and concentrated. Silica gel column chromatography afforded the title compound. ¹H NMR (400 MHz, DMSO-d₆) δ 9.96 (s, 1H), 8.77 (s, 1H), 7.50 (t, J=11.7 Hz, 1H), 7.40-6.97 (m, 7H), 5.44-5.26 (m, 1H), 5.08-4.66 (m, 3H), 4.60-4.11 (m, 3H), 3.12 (td, J=14.7, 8.2 Hz, 1H), 3.05-2.91 (m, 1H), 2.70-2.56 (m, 1H), 2.54-2.51 (m, 2H), 1.33 (dd, J=17.0, 6.9 Hz, 3H). MS (ESI⁺) m/z 638 (M+Na)⁺.

### Example 388

### methyl {[(1R)-3′-(2-{(4-fluorobenzyl) [(2S)-1,1,1-trifluoropropan-2-yl]amino}-2-oxoethyl)-2′,4′-dioxo-2,3-dihydrospiro[indene-1,5′-[1,3]oxazolidin]-5-yl]carbamoyl}sulfamate

To a solution of 2-((R)-5-amino-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide (40 mg, 0.083 mmol) in 1 ml tetrahydrofuran at −40° C. was added sulfurisocyanatidic chloride (100 μl, 0.100 mmol, freshly prepared 1.0 M tetrahydrofuran solution) and the solution turned yellow after addition. The mixture was stirred at −40° C. to −30° C. for 30 minutes and was treated with methanol (26.7 mg, 0.834 mmol). The mixture was stirred at room temperature for 1 hour and quenched with water. The aqueous layer was extracted with ethyl acetate, washed with brine, dried over MgSO₄, filtered and concentrated. Silica gel column chromatography afforded the title compound. ¹H NMR (500 MHz, DMSO-d₆) δ 9.10 (d, J=4.9 Hz, 0.3H), 8.72 (d, J=4.2 Hz, 0.7H), 7.57 (q, J=7.4 Hz, 1H), 7.48-7.07 (m, 6H), 5.50-5.26 (m, 0.7H), 5.13-4.67 (m, 2.3H), 4.54 (ddd, J=68.3, 29.9, 11.1 Hz, 1.3H), 4.21 (d, J=17.1 Hz, 0.7H), 4.04-3.89 (d, 3H), 3.21-2.85 (m, 2H), 2.73-2.41 (m, 2H), 1.44-1.28 (m, 3H). MS (ESI⁻) m/z 615 (M−H)⁻.

### Example 389

### 2-((R)-5-(3-(N-(cyclopropylmethyl)sulfamoyl)ureido)-2′,4′-dioxo-2,3-dihydrospiro[indene-1,5′-oxazolidine]-3′-yl)-N-(4-fluorobenzyl)-N—((S)-1,1,1-trifluoropropan-2-yl)acetamide

The title compound was prepared as described in EXAMPLE 387, substituting cyclopropylmethanamine for methanamine. ¹H NMR (400 MHz, DMSO-d₆) δ 9.96 (s, 1H), 8.75 (s, 1H), 7.69 (s, 0.7H), 7.53 (d, J=6.0 Hz, 1H), 7.27 (ddt, J=44.2, 17.5, 7.1 Hz, 5.3H), 5.40 (s, 0.7H), 4.89 (ddd, J=58.5, 41.0, 21.0 Hz, 2.3H), 4.63-4.38 (m, 1.3H), 4.21 (d, J=17.0 Hz, 0.7H), 3.23-2.56 (m, 6H), 1.43-1.17 (m, 3H), 0.90 (d, J=33.3 Hz, 1H), 0.39 (dd, J=11.3, 9.4 Hz, 2H), 0.15 (t, J=13.9 Hz, 2H). MS (ESI⁺) m/z 678 (M+Na)⁺.

### Example 390

### N-[(1S)-1-cyclopropylethyl]-N-(4-fluorobenzyl)-2-{7′-[(methylcarbamoyl)amino]-2,5-dioxo-3′,4′-dihydro-1H,1′H-spiro[imidazolidine-4,2′-naphthalen]-1-yl}acetamide

The title compound was prepared as described in EXAMPLE 282, substituting 2-(7′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆, 90° C.) δ ppm 8.38-8.14 (m, 1H), 8.07 (brs, 1H), 7.50-7.28 (m, 2H), 7.26-7.08 (m, 4H), 7.00 (d, J=8.2 Hz, 1H), 5.86-5.75 (m, 1H), 4.66 (brs, 2H), 4.36-4.13 (m, 2H), 3.58 (brs, 1H), 3.14 (d, J=16.9 Hz, 1H), 2.93-2.82 (m, 2H), 2.75 (d, J=16.8 Hz, 1H), 2.67 (d, J=4.3 Hz, 3H), 2.08-1.98 (m, 1H), 1.92-1.81 (m, 1H), 1.19 (d, J=6.8 Hz, 3H), 1.04-0.93 (m, 1H), 0.57-0.46 (m, 1H), 0.34-0.16 (m, 3H). MS (ESI⁺) m/z 544 (M+Na)⁺.

### Example 391

### 2-[7′-(acetylamino)-2,5-dioxo-3′,4′-dihydro-1H, 1′H-spiro[imidazolidine-4,2′-naphthalen]-1-yl]-N-[(1S)-1-cyclopropylethyl]-N-(4-fluorobenzyl)acetamide

The title compound was prepared as described in EXAMPLE 346, substituting 2-(7′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (400 MHz, DMSO-d₆, 90° C.) δ ppm 9.47 (brs, 1H), 8.26 (brs, 1H), 7.41-7.27 (m, 3H), 7.25 (d, J=2.1 Hz, 1H), 7.16-7.05 (m, 2H), 7.02 (d, J=8.2 Hz, 1H), 4.63 (brs, 2H), 4.34-4.09 (m, 2H), 3.53 (brs, 1H), 3.12 (d, J=16.8 Hz, 1H), 2.92-2.82 (m, 2H), 2.74 (d, J=16.8 Hz, 1H), 2.05-1.96 (m, 4H), 1.92-1.78 (m, 1H), 1.17-1.10 (m, 3H), 1.03-0.88 (m, 1H), 0.55-0.43 (m, 1H), 0.35-0.16 (m, 3H). MS (ESI⁺) m/z 529 (M+Na)⁺.

### Example 392

### (1R)-3′-(2-{benzyl[(1S)-1-cyclopropylethyl]amino}-2-oxoethyl)-N-(2-methylpropyl)-2′,4′-dioxo-2,3-dihydrospiro[indene-1,5′-[1,3]oxazolidine]-5-carboxamide

### Example 392A

### (R)-benzyl 3′-(2-(benzyl((S)-1-cyclopropylethyl)amino)-2-oxoethyl)-2′,4′-dioxo-2,3-dihydrospiro[indene-1,5′-oxazolidine]-5-carboxylate

To N-benzyl-2-[(1R)-5-bromo-2′,4′-dioxo-2,3-dihydro-3′H-spiro[indene-1,5′-[1,3]oxazolidin]-3′-yl]-N-[(1S)-1-cyclopropylethyl]acetamide (1.48 g, 2.87 mmol) and PdCl₂(1,1′-bis(diphenylphosphino)ferrocene) (0.063 g, 0.086 mmol) in a 50 ml pressure bottle was added n-butanol (20 ml) and triethylamine (1.201 ml, 8.62 mmol). The reactor was purged with argon several times followed by carbon monoxide and the mixture was heated at 105° C. for 22 hours at 60 psi. The solution was concentrated, redissolved in dichloromethane and methanol and treated with Darco® (100 mesh activated charcoal, Sigma-Aldrich). After standing for 30 minutes, the suspension was filtered through diatomaceous earth and concentrated to afford the title compound.

### Example 392B

### (R)-3′-(2-(benzyl((S)-1-cyclopropylethyl)amino)-2-oxoethyl)-2′,4′-dioxo-2,3-dihydrospiro[indene-1,5′-oxazolidine]-5-carboxylic acid

(R)-Benzyl 3′-(2-(benzyl((S)-1-cyclopropylethyl)amino)-2-oxoethyl)-2′,4′-dioxo-2,3-dihydrospiro[indene-1,5′-oxazolidine]-5-carboxylate (2.40 g, 4.21 mmol), 20% palladium hydroxide on carbon (250 mg, 0.2 mmol) and methanol (30 ml) were added to a 50 ml pressure bottle. The mixture was stirred for 2 hours at 30 psi hydrogen and 50° C. The solution was filtered and concentrated to give a semi-solid that was triturated with 1:1 heptane-ethyl ether. The solvent was decanted and the solid was dried under vacuum to afford the title compound.

### Example 392C

### (1R)-3′-(2-{benzyl[(1S)-1-cyclopropylethyl]amino}-2-oxoethyl)-N-(2-methylpropyl)-2′,4′-dioxo-2,3-dihydrospiro[indene-1,5′-[1,3]oxazolidine]-5-carboxamide

The title compound was prepared as described in EXAMPLE 306, substituting (R)-3′-(2-(benzyl((S)-1-cyclopropylethyl)amino)-2-oxoethyl)-2′,4′-dioxo-2,3-dihydrospiro[indene-1,5′-oxazolidine]-5-carboxylic acid for cyclopropanecarboxylic acid and 2-methylpropan-1-amine for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆) δ ppm 7.80 (s, 1H), 7.72 (d, J=8.0 Hz, 1H), 7.51 (d, J=8.0 Hz, 1H), 7.36-7.29 (m, 4H), 7.27-7.21 (m, 1H), 4.70 (s, 2H), 4.48-4.32 (m, 2H), 3.23 (dt, J=15.2, 7.2 Hz, 1H), 3.17-3.08 (m, 3H), 2.74 (ddd, J=14.7, 8.6, 6.1 Hz, 1H), 2.60-2.53 (m, 1H), 1.89 (dt, J=13.4, 6.7 Hz, 1H), 1.19 (d, J=6.8 Hz, 3H), 1.03-0.94 (m, 1H), 0.91 (d, J=6.7 Hz, 6H), 0.52 (q, J=5.5 Hz, 1H), 0.35-0.27 (m, 2H), 0.23 (q, J=6.2, 5.7 Hz, 1H); MS (APCI⁺) m/z 518 (M+H)⁺.

TABLE 17
The following EXAMPLEs were prepared essentially as described in EXAMPLE 392, substituting
2-methylpropan-1-amine with an appropriate amine in EXAMPLE 392C. Some products were purified
by silica gel column chromatography while others were purified by reverse-phase HPLC.
Accordingly, some EXAMPLEs were isolated as trifluoroacetic acid salts.

| Ex | Name | NMR | MS |
|---|---|---|---|
| 408 | (1R)-3′-(2- | ¹H NMR (501 MHz, DMSO-d₆) δ ppm 7.92 (dd, J = | (APCI⁺) |
| | {benzyl[(1S)-1- | 9.1, 5.4 Hz, 1H), 7.71-7.67 (m, 1H), 7.61 (ddd, J = | m/z 548 |
| | cyclopropylethyl]amino}- | 12.0, 7.9, 1.6 Hz, 1H), 7.40 (dd, J = 12.1, 8.0 Hz, 1H), | (M + H)⁺ |
| | 2-oxoethyl)- | 7.24 (d, J = 4.3 Hz, 2H), 7.18-7.03 (m, 3H), 4.61 (s, | |
| | N-[(2R)-1- | 1H), 4.55-4.45 (m, 1H), 4.37-4.12 (m, 2H), 3.65 | |
| | hydroxy-3- | (dddd, J = 8.7, 6.6, 4.4, 2.0 Hz, 1H), 3.43-3.28 (m, | |
| | methylbutan-2-yl]- | 2H), 3.13-3.03 (m, 1H), 2.96 (ddt, J = 16.6, 9.0, 4.5 | |
| | 2′,4′-dioxo-2,3- | Hz, 1H), 2.56 (tdd, J = 15.0, 8.6, 6.4 Hz, 1H), 2.47- | |
| | dihydrospiro[indene- | 2.39 (m, 1H), 1.74 (hd, J = 6.8, 4.8 Hz, 1H), 1.02 (dd, J = | |
| | 1,5′- | 37.8, 6.7 Hz, 3H), 0.81 (dq, J = 13.4, 5.2, 4.7 Hz, | |
| | [1,3]oxazolidine]- | 1H), 0.73 (ddd, J = 17.6, 6.8, 4.1 Hz, 6H), 0.41-0.32 | |
| | 5-carboxamide | (m, 1H), 0.10 (dddd, J = 19.2, 13.9, 10.0, 5.0 Hz, 3H) | |
| 549 | (1R)-3′-(2- | ¹H NMR (400 MHz, DMSO-d₆) δ ppm 7.79 (s, 1H), | (APCI⁺) |
| | {benzyl[(1S)-1- | 7.71 (d, J = 8.0 Hz, 1H), 7.50 (d, J = 8.0 Hz, 1H), 7.37- | m/z 476 |
| | cyclopropylethyl]amino}- | 7.29 (m, 4H), 7.27-7.19 (m, 1H), 4.70 (s, 2H), 4.40 | (M + H)⁺ |
| | 2-oxoethyl)- | (t, J = 13.6 Hz, 2H), 3.72-3.52 (m, 1H), 3.29-3.08 | |
| | N-methyl-2′,4′- | (m, 1H), 2.82 (s, 3H), 2.78-2.69 (m, 1H), 2.60-2.53 | |
| | dioxo-2,3-dihydrospiro[indene- | (m, 1H), 1.19 (d, J = 6.8 Hz, 3H), 1.04-0.93 (m, 1H), | |
| | 1,5′-[1,3]oxazolidine]- | 0.52 (dt, J = 8.8, 5.6 Hz, 1H), 0.34-0.20 (m, 3H) | |
| | 5-carboxamide | | |
| 550 | (1R)-3′-(2- | ¹H NMR (400 MHz, DMSO-d₆) δ ppm 7.80 (s, 1H), | ((APCI⁺) |
| | {benzyl[(1S)-1- | 7.75-7.69 (m, 1H), 7.50 (d, J = 8.1 Hz, 1H), 7.33 (d, J = | m/z 504 |
| | cyclopropylethyl]amino}- | 4.8 Hz, 4H), 7.25 (d, J = 7.5 Hz, 1H), 4.70 (s, 2H), | (M + H)⁺ |
| | 2-oxoethyl)- | 4.50-4.35 (m, 2H), 4.09 (p, J = 6.6 Hz, 1H), 3.27- | |
| | 2′,4′-dioxo-N- | 3.19 (m, 1H), 3.12 (ddd, J = 16.5, 8.5, 4.8 Hz, 1H), | |
| | (propan-2-yl)-2,3- | 2.74 (ddd, J = 14.7, 8.6, 6.2 Hz, 1H), 2.59-2.52 (m, | |
| | dihydrospiro[indene- | 1H), 1.20 (s, 3H), 1.19 (s, 3H), 1.07-0.90 (m, 1H), | |
| | 1,5′-[1,3]oxazolidine]- | 0.52 (dt, J = 8.2, 5.3 Hz, 1H), 0.30 (td, J = 7.6, 2.6 Hz, | |
| | 5-carboxamide | 2H), 0.24 (dq, J = 11.0, 5.6, 4.9 Hz, 1H) | |
| 551 | (1R)-3′-(2- | ¹H NMR (400 MHz, DMSO-d₆) δ ppm 7.81 (d, J = 1.4 | (APCI⁺) |
| | {benzyl[(1S)-1- | Hz, 1H), 7.74-7.70 (m, 1H), 7.51 (d, J = 8.1 Hz, 1H), | m/z 520 |
| | cyclopropylethyl]amino}- | 7.33 (d, J = 4.7 Hz, 4H), 7.25 (s, 1H), 4.70 (s, 2H), | (M + H)⁺ |
| | 2-oxoethyl)- | 4.50-4.34 (m, 2H), 3.68-3.55 (m, 1H), 3.53-3.49 | |
| | N-(2-methoxyethyl)- | (m, 2H), 3.45 (dd, J = 6.6, 4.3 Hz, 2H), 3.30 (s, 3H), | |
| | 2′,4′-dioxo-2,3- | 3.27-3.19 (m, 1H), 3.13 (ddd, J = 16.4, 8.6, 4.7 Hz, | |
| | dihydrospiro[indene- | 1H), 2.74 (ddd, J = 14.6, 8.6, 6.2 Hz, 1H), 2.59-2.52 | |
| | 1,5′-[1,3]oxazolidine]- | (m, 1H), 1.19 (d, J = 6.8 Hz, 3H), 0.99 (qd, J = 8.2, 7.5, | |
| | 5-carboxamide | 4.2 Hz, 1H), 0.52 (dt, J = 8.2, 5.3 Hz, 1H), 0.34-0.27 | |
| | | (m, 2H), 0.24 (p, J = 5.2 Hz, 1H) | |
| 552 | 3′-{2-[benzyl(1- | ¹H NMR (400 MHz, DMSO-d₆) δ ppm 7.81 (s, 1H), | (APCI⁺) |
| | cyclopropylethyl)amino]- | 7.73 (d, J = 8.1 Hz, 1H), 7.51 (d, J = 8.0 Hz, 1H), 7.36- | m/z 546 |
| | 2-oxoethyl}- | 7.29 (m, 4H), 7.27-7.20 (m, 1H), 4.70 (s, 2H), 4.50- | (M + H)⁺ |
| | 2′,4′-dioxo-N- | 4.34 (m, 2H), 4.01 (p, J = 6.3 Hz, 1H), 3.79 (dt, J = | |
| | (tetrahydrofuran-2- | 8.2, 6.4 Hz, 1H), 3.69-3.57 (m, 2H), 3.36 (d, J = 5.9 | |
| | ylmethyl)-2,3- | Hz, 2H), 3.23 (dt, J = 15.1, 7.3 Hz, 1H), 3.14 (td, J = | |
| | dihydrospiro[indene- | 8.4, 5.0 Hz, 1H), 2.74 (ddd, J = 14.7, 8.6, 6.1 Hz, 1H), | |
| | 1,5′-[1,3]oxazolidine]- | 2.61-2.53 (m, 1H), 1.98-1.77 (m, 4H), 1.66-1.55 | |
| | 5-carboxamide | (m, 1H), 1.19 (d, J = 6.8 Hz, 4H), 1.03-0.93 (m, 1H), | |
| | | 0.52 (dt, J = 8.3, 5.2 Hz, 1H), 0.34-0.27 (m, 2H), 0.24 | |
| | | (p, J = 5.4 Hz, 1H) | |
| 553 | 3′-(2-{benzyl[(1S)- | ¹H NMR (400 MHz, DMSO-d₆) δ ppm 7.83-7.78 (m, | (APCI⁺) |
| | 1-cyclopropylethyl]amino}- | 1H), 7.76-7.69 (m, 2H), 7.51 (d, J = 8.0 Hz, 1H), 7.37- | m/z 546 |
| | 2-oxoethyl)- | 7.30 (m, 4H), 7.27-7.21 (m, 1H), 4.70 (s, 2H), 4.51- | (M + H)⁺ |
| | 2′,4′-dioxo-N- | 4.34 (m, 2H), 3.79-3.70 (m, 2H), 3.64 (q, J = 7.6 | |
| | (tetrahydrofuran-3- | Hz, 2H), 3.47 (dd, J = 8.6, 5.7 Hz, 1H), 3.33-3.29 (m, | |
| | ylmethyl)-2,3- | 2H), 3.28-3.19 (m, 1H), 3.14 (td, J = 8.2, 4.8 Hz, 1H), | |
| | dihydrospiro[indene- | 2.74 (ddd, J = 14.7, 8.5, 6.2 Hz, 1H), 2.60-2.53 (m, | |
| | 1,5′-[1,3]oxazolidine]- | 1H), 2.02-1.90 (m, 1H), 1.63 (ddd, J = 14.4, 12.6, 6.7 | |
| | 5-carboxamide | Hz, 1H), 1.19 (d, J = 6.8 Hz, 3H), 1.03-0.93 (m, 1H), | |
| | | 0.52 (dt, J = 8.3, 5.3 Hz, 1H), 0.30 (td, J = 8.5, 8.0, 3.9 | |
| | | Hz, 2H), 0.23 (q, J = 6.1 Hz, 1H) | |
| 554 | (1R)-3′-(2- | ¹H NMR (400 MHz, DMSO-d₆) δ ppm 7.85 (d, J = 1.5 | (APCI⁺) |
| | {benzyl[(1S)-1- | Hz, 1H), 7.76 (dd, J = 8.2, 1.5 Hz, 1H), 7.55 (d, J = 8.1 | m/z 501 |
| | cyclopropylethyl]amino}- | Hz, 1H), 7.33 (d, J = 4.9 Hz, 4H), 7.28-7.20 (m, 1H), | (M + H)⁺ |
| | 2-oxoethyl)-N-(cyanomethyl)- | 4.70 (s, 2H), 4.51-4.34 (m, 2H), 4.27 (s, 2H), 3.31- | |
| | 2′,4′-dioxo-2,3- | 3.20 (m, 1H), 3.19-3.10 (m, 1H), 2.80-2.70 (m, 1H), | |
| | dihydrospiro[indene- | 2.61-2.53 (m, 1H), 1.19 (d, J = 6.7 Hz, 3H), 1.04- | |
| | 1,5′-[1,3]oxazolidine]- | 0.93 (m, 1H), 0.52 (dt, J = 8.2, 5.3 Hz, 1H), 0.34-0.19 | |
| | 5-carboxamide | (m, 3H) | |
| 555 | (1R)-3′-(2- | ¹H NMR (400 MHz, DMSO-d₆) δ ppm 7.82-7.78 (m, | (APCI⁺) |
| | {benzyl[(1S)-1- | 1H), 7.74-7.70 (m, 3H), 7.51 (d, J = 8.0 Hz, 1H), 7.33 | m/z 520 |
| | cyclopropylethyl]amino}- | (d, J = 4.7 Hz, 4H), 7.27-7.21 (m, 1H), 4.70 (s, 2H), | (M + H)⁺ |
| | 2-oxoethyl)- | 4.49-4.34 (m, 2H), 3.52 (t, J = 6.3 Hz, 2H), 3.36 (t, J = | |
| | N-(3-hydroxypropyl)- | 6.9 Hz, 2H), 3.27-3.09 (m, 1H), 2.79-2.70 (m, | |
| | 2′,4′-dioxo-2,3- | 1H), 2.61-2.53 (m, 1H), 1.74 (p, J = 6.6 Hz, 2H), 1.19 | |
| | dihydrospiro[indene- | (d, J = 6.8 Hz, 3H), 1.03-0.94 (m, 1H), 0.57-0.47 | |
| | 1,5′-[1,3]oxazolidine]- | (m, 1H), 0.30 (t, J = 6.5 Hz, 2H), 0.26-0.19 (m, 1H) | |
| | 5-carboxamide | | |
| 556 | 3′-{2-[benzyl(1- | ¹H NMR (400 MHz, DMSO-d₆) δ ppm 7.81 (d, J = 1.4 | (APCI⁺) |
| | cyclopropylethyl)amino]- | Hz, 1H), 7.75-7.71 (m, 1H), 7.51 (d, J = 8.0 Hz, 1H), | m/z 548 |
| | 2-oxoethyl}- | 7.36-7.30 (m, 4H), 7.27-7.20 (m, 1H), 4.70 (s, 2H), | (M + H)⁺ |
| | N-(1-hydroxy-3- | 4.49-4.34 (m, 2H), 3.81 (q, J = 5.7 Hz, 1H), 3.68- | |
| | methylbutan-2-yl)- | 3.53 (m, 3H), 3.28-3.08 (m, 1H), 2.78-2.70 (m, 1H), | |
| | 2′,4′-dioxo-2,3- | 2.60-2.53 (m, 1H), 1.95 (q, J = 6.8 Hz, 1H), 1.19 (d, J = | |
| | dihydrospiro[indene- | 6.8 Hz, 3H), 1.06-0.88 (m, 9H), 0.56-0.48 (m, | |
| | 1,5′-[1,3]oxazolidine]- | 1H), 0.34-0.28 (m, 2H), 0.26-0.20 (m, 1H) | |
| | 5-carboxamide | | |
| 557 | N-(1-amino-4- | ¹H NMR (400 MHz, DMSO-d₆) δ ppm 7.85 (s, 1H), | (APCI⁺) |
| | methyl-1- | 7.77 (d, J = 8.2 Hz, 1H), 7.53 (d, J = 8.0 Hz, 1H), 7.38- | m/z 575 |
| | oxopentan-2-yl)-3′- | 7.30 (m, 4H), 7.28-7.21 (m, 1H), 4.70 (s, 2H), 4.51- | (M + H)⁺ |
| | {2-[benzyl(1- | 4.36 (m, 3H), 3.61 (s, 1H), 3.29-3.11 (m, 1H), 2.80- | |
| | cyclopropylethyl)amino]- | 2.70 (m, 1H), 2.61-2.52 (m, 1H), 1.74-1.63 (m, | |
| | 2-oxoethyl}- | 3H), 1.19 (d, J = 6.7 Hz, 3H), 1.04-0.95 (m, 1H), 0.92 | |
| | 2′,4′-dioxo-2,3- | (dd, J = 8.4, 6.1 Hz, 6H), 0.52 (d, J = 8.5 Hz, 1H), 0.30 | |
| | dihydrospiro[indene- | (t, J = 6.6 Hz, 2H), 0.27-0.18 (m, 1H) | |
| | 1,5′-[1,3]oxazolidine]- | | |
| | 5-carboxamide | | |
| 558 | (1R)-3′-(2- | ¹H NMR (400 MHz, DMSO-d₆) δ ppm 7.83 (d, J = 1.4 | (APCI⁺) |
| | {benzyl[(1S)-1- | Hz, 1H), 7.78-7.74 (m, 1H), 7.56 (d, J = 8.1 Hz, 1H), | m/z 561 |
| | cyclopropylethyl]amino}- | 7.37-7.28 (m, 4H), 7.28-7.21 (m, 1H), 4.70 (s, 2H), | (M + H)⁺ |
| | 2-oxoethyl)- | 4.51-4.34 (m, 2H), 3.65 (t, J = 6.4 Hz, 3H), 3.31- | |
| | N-[2-(diethylamino)ethyl]- | 3.22 (m, 3H), 3.20 (q, J = 7.0 Hz, 5H), 3.16-3.09 (m, | |
| | 2′,4′-dioxo-2,3- | 1H), 2.76 (ddd, J = 14.6, 8.5, 6.1 Hz, 1H), 2.57 (ddd, J = | |
| | dihydrospiro[indene- | 14.6, 8.4, 4.8 Hz, 1H), 1.26 (t, J = 7.2 Hz, 6H), 1.19 | |
| | 1,5′-[1,3]oxazolidine]- | (d, J = 6.8 Hz, 3H), 1.03-0.93 (m, 1H), 0.56-0.48 | |
| | 5-carboxamide | (m, 1H), 0.34-0.27 (m, 2H), 0.26-0.20 (m, 1H) | |
| 559 | (1R)-3′-(2- | ¹H NMR (400 MHz, DMSO-d₆) δ ppm 8.81 (s, 1H), | (APCI⁺) |
| | {benzyl[(1S)-1- | 7.80 (s, 1H), 7.73 (d, J = 8.2 Hz, 1H), 7.62 (s, 1H), | m/z 570 |
| | cyclopropylethyl]amino}- | 7.53 (d, J = 8.1 Hz, 1H), 7.49 (s, 1H), 7.36-7.30 (m, | (M + H)⁺ |
| | 2-oxoethyl)- | 4H), 7.27-7.21 (m, 1H), 4.70 (s, 2H), 4.51-4.37 (m, | |
| | N-[3-(1H-imidazol- | 2H), 4.26 (t, J = 7.0 Hz, 2H), 3.35 (t, J = 6.6 Hz, 2H), | |
| | 1-yl)propyl]-2′,4′- | 3.29-3.20 (m, 1H), 3.16 (dd, J = 8.6, 4.9 Hz, 1H), | |
| | dioxo-2,3- | 2.93-2.84 (m, 1H), 2.80-2.71 (m, 1H), 2.61-2.53 | |
| | dihydrospiro[indene- | (m, 1H), 2.14 (p, J = 6.8 Hz, 2H), 1.19 (d, J = 6.8 Hz, | |
| | 1,5′-[1,3]oxazolidine]- | 3H), 1.05-0.93 (m, 1H), 0.56-0.48 (m, 1H), 0.30 (t, | |
| | 5-carboxamide | J = 6.2 Hz, 2H), 0.26-0.20 (m, 1H) | |
| 560 | (1R)-3′-(2- | ¹H NMR (400 MHz, DMSO-d₆) δ ppm 7.81 (s, 1H), | (APCI⁺) |
| | {benzyl[(1S)-1- | 7.75-7.71 (m, 1H), 7.51 (d, J = 8.1 Hz, 1H), 7.33 (d, J = | m/z 587 |
| | cyclopropylethyl]amino}- | 4.8 Hz, 4H), 7.27-7.21 (m, 1H), 4.70 (s, 2H), 4.50- | (M + H)⁺ |
| | 2-oxoethyl)- | 4.33 (m, 2H), 3.40-3.34 (m, 2H), 3.27 (dt, J = 8.8, 6.9 | |
| | 2′,4′-dioxo-N-[3-(2- | Hz, 4H), 3.23-3.18 (m, 1H), 3.17-3.10 (m, 1H), 2.79- | |
| | oxopyrrolidin-1- | 2.69 (m, 1H), 2.60-2.53 (m, 1H), 2.23 (t, J = 8.1 | |
| | yl)propyl]-2,3- | Hz, 2H), 1.94 (p, J = 7.5 Hz, 2H), 1.77 (p, J = 7.0 Hz, | |
| | dihydrospiro[indene- | 2H), 1.19 (d, J = 6.7 Hz, 3H), 1.03-0.93 (m, 1H), 0.56- | |
| | 1,5′-[1,3]oxazolidine]- | 0.48 (m, 1H), 0.34-0.27 (m, 2H), 0.25-0.20 (m, | |
| | 5-carboxamide | 1H) | |

### Example 406

### 2-(5-bromo-4-fluoro-2′,4′-dioxo-2,3-dihydro-3′H-spiro[indene-1,5′-[1,3]oxazolidin]-3′-yl)-N-[(1S)-1-cyclopropyl-2,2,2-trifluoroethyl]-N-(4-fluorobenzyl)acetamide

### Example 406A

### 3-(3-bromo-2-fluorophenyl)propanoic acid

To a flask charged with triethylamine (8.24 ml, 59.1 mmol) was added formic acid (1.12 ml, 29.2 mmol) portionwise and the mixture was stirred for 15 minutes at room temperature. The mixture was diluted with N,N-dimethylformamide (37.5 ml). To the solution was added 3-bromo-2-fluorobenzaldehyde (10 g, 49.3 mmol) and 2,2-dimethyl-1,3-dioxane-4,6-dione (7.10 g, 49.3 mmol). The mixture was heated to 100° C. overnight and then poured into ice/concentrated hydrochloric acid (400 ml). The mixture was extracted with dichloromethane and the combined organic layers were washed with 1M aqueous sodium hydroxide. The aqueous layer was acidified to pH=2 with concentrated hydrochloric acid and extracted with ethyl acetate. The organic layer was dried with MgSO₄ and concentrated under reduced pressure to give the crude title compound which was used in the next step without purification.

### Example 406B

### 3-(3-bromo-2-fluorophenyl)propanoyl chloride

A mixture of 3-(3-bromo-2-fluorophenyl)propanoic acid (11 g, 44.5 mmol) in dichloromethane (100 ml) was treated with N,N-dimethylformamide (0.172 ml, 2.226 mmol) and oxalyl chloride (15.19 ml, 178 mmol). The mixture was stirred at room temperature for 0.5 hours, then concentrated to dryness to give the title compound which was used directly in the next step.

### Example 406C

### 5-bromo-4-fluoro-2,3-dihydro-1H-inden-1-one

A solution of crude 3-(3-bromo-2-fluorophenyl)propanoyl chloride (11 g, 41.4 mmol) in dichloromethane (150 ml) was added dropwise to a refluxing suspension of AlCl₃ (8.29 g, 62.1 mmol) in dichloromethane (200 ml). The mixture was refluxed for 1.5 hours, poured into ice/concentrated hydrochloric acid and extracted with dichloromethane. The organic layers were combined and concentrated under reduced pressure. The residue was purified by silica gel column chromatography to afford the title compound.

### Example 406D

### 5-bromo-4-fluoro-2,3-dihydrospiro[indene-1,5′-oxazolidine]-2′,4′-dione

The title compound was prepared as described in EXAMPLE 301A-D, substituting 5-bromo-4-fluoro-2,3-dihydro-1H-inden-1-one for 7-bromochroman-4-one in EXAMPLE 301A.

### Example 406E

### (S)—N-benzyl-2-bromo-N-(1-cyclopropyl-2,2,2-trifluoroethyl)acetamide

The title compound was prepared as described in EXAMPLE 281A-B, substituting (S)-1-cyclopropyl-2,2,2-trifluoroethanamine for (S)-1,1,1-trifluoropropan-2-amine in EXAMPLE 281A and (S)-1-cyclopropyl-2,2,2-trifluoro-N-(4-fluorobenzyl)ethanamine for (S)-1,1,1-trifluoro-N-(4-fluorobenzyl)propan-2-amine in EXAMPLE 281B.

### Example 406F

### 2-(5-bromo-4-fluoro-2′,4′-dioxo-2,3-dihydro-3′H-spiro[indene-1,5′-[1,3]oxazolidin]-3′-yl)-N-[(1S)-1-cyclopropyl-2,2,2-trifluoroethyl]-N-(4-fluorobenzyl)acetamide

The title compound was prepared as described in EXAMPLE 281F, substituting 5-bromo-4-fluoro-2,3-dihydrospiro[indene-1,5′-oxazolidine]-2′,4′-dione for (R)-5-bromo-2,3-dihydrospiro[indene-1,5′-oxazolidine]-2′,4′-dione and (S)—N-benzyl-2-bromo-N-(1-cyclopropyl-2,2,2-trifluoroethyl)acetamide for (S)-2-bromo-N-(4-fluorobenzyl)-N-(1,1,1-trifluoropropan-2-yl)acetamide. ¹H NMR (400 MHz, DMSO-d₆) δ 7.80-7.01 (m, 6H), 4.90 (ddd, J=29.0, 22.5, 12.4 Hz, 2H), 4.64-4.10 (m, 3H), 3.27-3.05 (m, 2H), 2.76-2.52 (m, 2H), 1.22 (s, 1H), 0.73 (d, J=5.7 Hz, 2H), 0.37 (s, 1H), 0.24 (s, 1H). MS (ESI⁺) m/z 611 (M+Na)⁺.

### Example 407

### 2-(5-bromo-6-fluoro-2′,4′-dioxo-2,3-dihydro-3′H-spiro[indene-1,5′-[1,3]oxazolidin]-3′-yl)-N-[(1S)-1-cyclopropyl-2,2,2-trifluoroethyl]-N-(4-fluorobenzyl)acetamide

### Example 407A

### 3-(3-bromo-4-fluorophenyl)propanoic acid

To a flask charged with triethylamine (8.24 ml, 59.1 mmol) was added formic acid (1.12 ml, 29.2 mmol) portionwise and the mixture was stirred for 15 minutes at room temperature. The mixture was diluted with N,N-dimethylformamide (37.5 ml). To the solution was added 3-bromo-2-fluorobenzaldehyde (10 g, 49.3 mmol) and 2,2-dimethyl-1,3-dioxane-4,6-dione (7.10 g, 49.3 mmol). The mixture was heated to 100° C. overnight and then poured into ice/concentrated hydrochloric acid (400 ml). The mixture was extracted with dichloromethane. The combined organic layers were washed with 1M aqueous sodium hydroxide. The aqueous layer was acidified to pH=2 with concentrated hydrochloric acid and extracted with ethyl acetate. The organic layer was dried with MgSO₄ and concentrated under reduced pressure to give the crude title compound which was used in the next step without purification.

### Example 407B

### 3-(3-bromo-4-fluorophenyl)propanoyl chloride

A mixture of 3-(3-bromo-4-fluorophenyl)propanoic acid (11 g, 44.5 mmol) in dichloromethane (100 ml) was treated with N,N-dimethylformamide (0.172 ml, 2.226 mmol) and oxalyl chloride (15.19 ml, 178 mmol). The mixture was stirred at room temperature for 0.5 hour, then concentrated to dryness to give the title compound, which was used directly in next step.

### Example 407C

### 5-bromo-6-fluoro-2,3-dihydro-1H-inden-1-one and 7-bromo-6-fluoro-2,3-dihydro-1H-inden-1-one

A solution of crude 3-(3-bromo-4-fluorophenyl)propanoyl chloride (11 g, 41.4 mmol) in dichloromethane (150 ml) was added dropwise to a refluxing suspension of AlCl₃ (8.29 g, 62.1 mmol) in dichloromethane (200 ml). The mixture was refluxed for 1.5 hours and poured into ice/concentrated hydrochloric acid and extracted with dichloromethane. The organic layers were combined and concentrated under reduced pressure. The residue was purified by silica gel column chromatography to afford 5-bromo-6-fluoro-2,3-dihydro-1H-inden-1-one (major product) and 7-bromo-6-fluoro-2,3-dihydro-1H-inden-1-one (minor product).

### Example 407D

### 5-bromo-6-fluoro-2,3-dihydrospiro[indene-1,5′-oxazolidine]-2′,4′-dione

The title compound was prepared as described in EXAMPLE 301A-D, substituting 5-bromo-6-fluoro-2,3-dihydro-1H-inden-1-one for 7-bromochroman-4-one in EXAMPLE 301A.

### Example 407E

### 2-(5-bromo-6-fluoro-2′,4′-dioxo-2,3-dihydro-3′H-spiro[indene-1,5′-[1,3]oxazolidin]-3′-yl)-N-[(1S)-1-cyclopropyl-2,2,2-trifluoroethyl]-N-(4-fluorobenzyl)acetamide

The title compound was prepared as described in EXAMPLE 281F, substituting 5-bromo-6-fluoro-2,3-dihydrospiro[indene-1,5′-oxazolidine]-2′,4′-dione for (R)-5-bromo-2,3-dihydrospiro[indene-1,5′-oxazolidine]-2′,4′-dione and (S)-2-bromo-N-(1-cyclopropyl-2,2,2-trifluoroethyl)-N-(4-fluorobenzyl)acetamide for (S)-2-bromo-N-(4-fluorobenzyl)-N-(1,1,1,1-trifluoropropan-2-yl)acetamide. ¹H NMR (400 MHz, DMSO-d₆) δ 7.82 (d, J=6.4 Hz, 1H), 7.52-7.03 (m, 5H), 5.11-4.07 (m, 5H), 3.25-2.98 (m, 2H), 2.77-2.54 (m, 2H), 1.24 (s, 1H), 0.75 (dd, J=22.3, 16.1 Hz, 2H), 0.32 (d, J=45.5 Hz, 2H). MS (ESI⁺) m/z 609.0 (M+Na)⁺.

### Example 410

### N-(4-fluorobenzyl)-2-(2′-methyl-2,5-dioxo-2′,3′-dihydro-1H-spiro[imidazolidine-4,1′-inden]-1-yl)-N-[(2S)-1,1,1-trifluoropropan-2-yl]acetamide

### Example 410A

### 2′-methyl-2′,3′-dihydrospiro[imidazolidine-4,1′-indene]-2,5-dione

The title compound was prepared as described in EXAMPLE 290B, substituting 2-methyl-2,3-dihydro-1H-inden-1-one for 6-bromobenzo[b]thiophen-3 (2H)-one 1,1-dioxide.

### Example 410B

### N-(4-fluorobenzyl)-2-(2′-methyl-2,5-dioxo-2′,3′-dihydro-1H-spiro[imidazolidine-4,1′-inden]-1-yl)-N-[(2S)-1,1,1-trifluoropropan-2-yl]acetamide

The title compound was prepared as described in EXAMPLE 281F, substituting 2′-methyl-2′,3′-dihydrospiro[imidazolidine-4,1′-indene]-2,5-dione for (R)-5-bromo-2,3-dihydrospiro[indene-1,5′-oxazolidine]-2′,4′-dione. ¹H NMR (400 MHz, DMSO-d₆) δ 8.92-8.49 (m, 1H), 7.56-6.86 (m, 8H), 5.60-3.93 (m, 5H), 3.26-2.51 (m, 3H), 1.50-0.91 (m, 6H). MS (ESI) m/e 500 (M+Na)⁺.

### Example 411

### 2-(5-amino-4-fluoro-2′,4′-dioxo-2,3-dihydro-3′H-spiro[indene-1,5′-[1,3]oxazolidin]-3′-yl)-N-[(1S)-1-cyclopropyl-2,2,2-trifluoroethyl]-N-(4-fluorobenzyl)acetamide

The title compound was prepared as described in EXAMPLE 284D-E, substituting 2-(5-bromo-4-fluoro-2′,4′-dioxo-2,3-dihydro-3′H-spiro[indene-1,5′-[1,3]oxazolidin]-3′-yl)-N-[(1S)-1-cyclopropyl-2,2,2-trifluoroethyl]-N-(4-fluorobenzyl)acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide. ¹H NMR (400 MHz, DMSO-d₆) δ 7.51-6.63 (m, 6H), 5.49 (s, 2H), 5.10-4.07 (m, 5H), 3.15-2.94 (m, 2H), 2.72-2.50 (m, 2H), 1.24 (s, 1H), 0.87-0.71 (m, 2H), 0.31 (dd, J=47.3, 4.6 Hz, 2H). MS (ESI⁺) m/z 546 (M+Na)⁺.

### Example 412

### N-[(1S)-1-cyclopropyl-2,2,2-trifluoroethyl]-N-(4-fluorobenzyl)-2-{4-fluoro-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 282, substituting 2-(5-amino-4-fluoro-2′,4′-dioxo-2,3-dihydro-3′H-spiro[indene-1,5′-[1,3]oxazolidin]-3′-yl)-N-[(1S)-1-cyclopropyl-2,2,2-trifluoroethyl]-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆) δ 8.47 (s, 1H), 8.10 (t, J=7.8 Hz, 1H), 7.51-7.04 (m, 6H), 6.54 (d, J=4.6 Hz, 1H), 5.09-4.11 (m, 5H), 3.20-3.07 (m, 2H), 2.75-2.55 (m, 2H), 1.24 (s, 1H), 0.86-0.65 (m, 2H), 0.31 (d, J=46.7 Hz, 2H). MS (ESI⁺) m/z 603 (M+H)⁺.

### Example 423

### N-[1-(2-{[(1S)-1-cyclopropylethyl](4-fluorobenzyl)amino}-2-oxoethyl)-2,5-dioxo-3′,4′-dihydro-1′H-spiro[imidazolidine-4,2′-naphthalen]-7′-yl]-5-oxo-D-prolinamide

The title compound was prepared as described in EXAMPLE 306, substituting (R)-5-oxopyrrolidine-2-carboxylic acid for cyclopropanecarboxylic acid and 2-(7′-amino-2,5-dioxo-3′,4′-dihydro-1H, 1′H-spiro[imidazolidine-4,2′-naphthalen]-1-yl)-N-[(1S)-1-cyclopropylethyl]-N-(4-fluorobenzyl)acetamide for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆, 90° C.) δ ppm 9.54 (s, 1H), 8.26 (brs, 1H), 7.73-7.48 (m, 1H), 7.41-7.27 (m, 4H), 7.16-7.01 (m, 3H), 4.63 (brs, 2H), 4.26-4.12 (m, 3H), 3.81-3.29 (m, 1H), 3.13 (d, J=16.8 Hz, 1H), 2.89 (dd, J=7.9, 5.4 Hz, 2H), 2.76 (d, J=16.9 Hz, 1H), 2.39-1.97 (m, 5H), 1.92-1.79 (m, 1H), 1.17-1.10 (m, 3H), 1.03-0.88 (m, 1H), 0.53-0.45 (m, 1H), 0.35-0.18 (m, 3H). MS (ESI⁺) m/z 576 (M+H)⁺.

### Example 424

### 2-{7-[1-(2-amino-2-oxoethyl)-1H-pyrazol-4-yl]-2′,4′-dioxo-2,3-dihydro-3′H-spiro[chromene-4,5′-[1,3]oxazolidin]-3′-yl}-N-benzyl-N-[(1S)-1-cyclopropylethyl]acetamide

The title compound was prepared as described in EXAMPLE 295, substituting N-benzyl-2-(7-bromo-2′,4′-dioxo-2,3-dihydro-3′H-spiro[chromene-4,5′-[1,3]oxazolidin]-3′-yl)-N-[(1S)-1-cyclopropylethyl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆, 90° C.) δ ppm 8.11 (s, 1H), 7.85 (s, 1H), 7.46-7.20 (m, 6H), 7.17 (dd, J=8.2, 1.8 Hz, 1H), 7.13-6.90 (m, 3H), 4.79-4.68 (m, 4H), 4.56-4.24 (m, 3H), 4.19 (ddd, J=11.4, 10.0, 3.5 Hz, 1H), 3.87-3.28 (m, 1H), 2.53-2.36 (m, 2H), 1.19 (d, J=7.1 Hz, 3H), 1.05-0.93 (m, 1H), 0.59-0.44 (m, 1H), 0.38-0.18 (m, 3H). MS (ESI⁺) m/z 558 (M+H)⁺.

### Example 425

### 2-{7′-[1-(2-amino-2-oxoethyl)-1H-pyrazol-4-yl]-2,5-dioxo-3′,4′-dihydro-1H,1′H-spiro[imidazolidine-4,2′-naphthalen]-1-yl}-N-[(1S)-1-cyclopropylethyl]-N-(4-fluorobenzyl)acetamide

The title compound was prepared as described in EXAMPLE 295, substituting 2-(7′-bromo-2,5-dioxo-3′,4′-dihydro-1H, 1′H-spiro[imidazolidine-4,2′-naphthalen]-1-yl)-N-[(1S)-1-cyclopropylethyl]-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ ppm 8.65 (d, J=15.6 Hz, 1H), 8.06 (d, J=1.7 Hz, 1H), 7.83 (s, 1H), 7.52-7.40 (m, 2H), 7.40-7.31 (m, 2H), 7.31-7.16 (m, 3H), 7.16-7.04 (m, 2H), 4.78-4.67 (m, 3H), 4.67-4.49 (m, 1H), 4.44-4.19 (m, 1H), 4.21-4.08 (m, 1H), 3.41-3.23 (m, 1H), 3.18 (dd, J=16.9, 7.0 Hz, 1H), 3.00-2.88 (m, 2H), 2.81 (dd, J=17.1, 5.5 Hz, 1H), 2.13-1.96 (m, 1H), 1.92-1.80 (m, 1H), 1.22-1.05 (m, 3H), 1.00-0.85 (m, 1H), 0.56-0.42 (m, 1H), 0.41-0.06 (m, 3H). MS (ESI⁺) m/z 573 (M+H)⁺.

### Example 428

### 2-(5′-bromo-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

### Example 428A

### 5′-bromo-2′,3′-dihydrospiro[imidazolidine-4,1′-indene]-2,5-dione

The title compound was prepared as described in EXAMPLE 290B, substituting 5-bromo-2,3-dihydro-1H-inden-1-one for 6-bromobenzo[b]thiophen-3 (2H)-one 1,1-dioxide.

### Example 428B

### 5′-bromospiro[imidazolidine-4,1′-indene]-2,3′,5(2′H)-trione

5′-Bromo-2′,3′-dihydrospiro[imidazolidine-4,1′-indene]-2,5-dione (281 mg, 1.000 mmol) in 5 mL acetone was cooled to −78° C. KMnO₄ (1580 mg, 10.00 mmol) and ferric chloride (405 mg, 2.499 mmol) were added. The mixture was stirred at −78° C. for 2 hours and at room temperature overnight. The suspension was filtered and the solid was rinsed with acetone. The filtrate was concentrated and purified by silica gel column chromatography to give the title compound.

### Example 428C

### 2-(5′-bromo-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

The title compound was prepared as described in EXAMPLE 281F, substituting 5′-bromospiro[imidazolidine-4,1′-indene]-2,3′,5(2′H)-trione for (R)-5-bromo-2,3-dihydrospiro[indene-1,5′-oxazolidine]-2′,4′-dione. ¹H NMR (400 MHz, DMSO-d₆) δ 8.97 (d, J=7.7 Hz, 1H), 8.14-7.83 (m, 2H), 7.80-7.60 (m, 1H), 7.40-6.88 (m, 4H), 5.35 (h, J=7.7 Hz, 1H), 4.99-4.00 (m, 4H), 3.31-2.88 (m, 2H), 1.51-1.20 (m, 3H). MS (ESI⁺) m/z 558 (M+H)⁺.

### Example 429

### 2-(5-amino-6-fluoro-2′,4′-dioxo-2,3-dihydro-3′H-spiro[indene-1,5′-[1,3]oxazolidin]-3′-yl)-N-[(1S)-1-cyclopropyl-2,2,2-trifluoroethyl]-N-(4-fluorobenzyl)acetamide

The title compound was prepared as described in EXAMPLE 284D-E, substituting 2-(5-bromo-6-fluoro-2′,4′-dioxo-2,3-dihydro-3′H-spiro[indene-1,5′-[1,3]oxazolidin]-3′-yl)-N-[(1S)-1-cyclopropyl-2,2,2-trifluoroethyl]-N-(4-fluorobenzyl)acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide. ¹H NMR (400 MHz, DMSO-d₆) δ 7.52-6.92 (m, 5H), 6.67 (d, J=8.3 Hz, 1H), 5.51 (s, 2H), 5.08-4.11 (m, 5H), 2.94 (m, 2H), 2.70-2.33 (m, 4H), 1.22 (s, 1H), 0.91-0.59 (m, 2H), 0.48-0.10 (m, 2H). MS (ESI⁺) 546 (M+Na)⁺.

### Example 430

### N-[(1S)-1-cyclopropyl-2,2,2-trifluoroethyl]-N-(4-fluorobenzyl)-2-{6-fluoro-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 282, substituting 2-(5-amino-6-fluoro-2′,4′-dioxo-2,3-dihydro-3′H-spiro[indene-1,5′-[1,3]oxazolidin]-3′-yl)-N-[(1S)-1-cyclopropyl-2,2,2-trifluoroethyl]-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆) δ 8.44 (d, J=2.6 Hz, 1H), 8.17 (d, J=7.3 Hz, 1H), 7.48-6.98 (m, 4H), 6.55 (d, J=4.7 Hz, 1H), 5.10-4.00 (m, 5H), 2.90-3.20 (m, 2H), 2.70-2.42 (m, 5H), 1.22 (s, 1H), 0.77 (dd, J=35.6, 11.4 Hz, 2H), 0.31 (d, J=46.7 Hz, 2H). MS (ESI⁺) m/z 581 (M+H)⁺.

### Example 431

### N-[(1S)-1-cyclopropylethyl]-N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]acetamide

The title compound was prepared as described in EXAMPLE 295, substituting 2-((R)-5-bromo-2′,4′-dioxo-2,3-dihydrospiro[indene-1,5′-oxazolidin]-3′-yl)-N—((S)-1-cyclopropylethyl)-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-bromo-1H-pyrazol-1-yl)-N-methylacetamide for 5-(4,4,5,5-tetramethyl-1,3,2-dioxaborolan-2-yl)pyridin-2-ol. ¹H NMR (400 MHz, DMSO-d₆, 90° C.) δ ppm 8.11 (s, 1H), 7.86 (s, 1H), 7.77-7.60 (m, 1H), 7.57 (s, 1H), 7.51 (dd, J=8.0, 1.6 Hz, 1H), 7.48-7.27 (m, 3H), 7.27-6.98 (m, 2H), 4.76 (s, 2H), 4.69 (brs, 2H), 4.62-4.24 (m, 2H), 3.85-3.26 (m, 1H), 3.26-3.14 (m, 1H), 3.14-3.03 (m, 1H), 2.78-2.62 (m, 4H), 2.62-2.50 (m, 1H), 1.22-1.17 (m, 3H), 1.05-0.90 (m, 1H), 0.60-0.45 (m, 1H), 0.40-0.19 (m, 3H). MS (ESI⁺) m/z 574 (M+H)⁺.

### Example 432

### N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide

### Example 432A

### 2-((R)-2′,4′-dioxo-5-(4,4,5,5-tetramethyl-1,3,2-dioxaborolan-2-yl)-2,3-dihydrospiro[indene-1,5′-oxazolidin]-3′-yl)-N-(4-fluorobenzyl)-N—((S)-1,1,1-trifluoropropan-2-yl)acetamide

The title compound was prepared as described in EXAMPLE 353A, substituting 2-((R)-5-bromo-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for 2-((R)-5-bromo-2′,4′-dioxo-2,3-dihydrospiro[indene-1,5′-oxazolidin]-3′-yl)-N—((S)-1-cyclopropylethyl)-N-(4-fluorobenzyl)acetamide.

### Example 432B

### N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide

A mixture of 2-((R)-2′,4′-dioxo-5-(4,4,5,5-tetramethyl-1,3,2-dioxaborolan-2-yl)-2,3-dihydrospiro[indene-1,5′-oxazolidin]-3′-yl)-N-(4-fluorobenzyl)-N—((S)-1,1,1-trifluoropropan-2-yl)acetamide (0.03 g, 0.051 mmol), 2-(4-bromo-1H-pyrazol-1-yl)-N-methylacetamide (0.014 g, 0.066 mmol), sodium hydrogencarbonate (0.021 g, 0.254 mmol), and PdCl₂(1,1′-bis(diphenylphosphino)ferrocene)-dichloromethane adduct (3.32 mg, 4.07 μmol) in degassed dioxane (0.5 ml) and water (0.12 mL) was sealed under nitrogen and heated at 85° C. for 6 hours. The reaction mixture was cooled to room temperature, diluted with ethyl acetate and filtered through a bed of diatomaceous earth. The filtrate was concentrated and purified by reverse-phase HPLC to afford the title compound. ¹H NMR (400 MHz, DMSO-d₆, 90° C.) δ ppm 8.11 (s, 1H), 7.86 (s, 1H), 7.72-7.61 (m, 1H), 7.57 (d, J=1.5 Hz, 1H), 7.51 (dd, J=8.0, 1.6 Hz, 1H), 7.40 (d, J=8.1 Hz, 1H), 7.38-7.26 (m, 2H), 7.21-7.08 (m, 2H), 5.36-5.05 (m, 1H), 4.86 (d, J=17.6 Hz, 1H), 4.76 (s, 2H), 4.72-4.54 (m, 2H), 4.54-4.29 (m, 1H), 3.28-3.14 (m, 1H), 3.14-3.03 (m, 1H), 2.77-2.62 (m, 4H), 2.58-2.49 (m, 1H), 1.37 (d, J=7.0 Hz, 3H). MS (ESI⁺) m/z 602 (M+H)⁺.

TABLE 18
The following EXAMPLEs were prepared essentially as described in EXAMPLE 432B, substituting
2-(4-bromo-1H-pyrazol-1-yl)-N-methylacetamide with an appropriate aryl halide. Some products
were purified by flash chromatography while others were purified by reverse-phase HPLC.
Accordingly, some EXAMPLEs were isolated as trifluoroacetic acid salts.

| Ex | Name | NMR | MS |
|---|---|---|---|
| 433 | 2-[(1R)-5-{1-[2- | ¹H NMR (400 MHz, DMSO-d₆, 120° C.) δ ppm | (ESI(+)) |
| | (dimethylamino)-2- | 8.01 (s, 1H), 7.80 (s, 1H), 7.54 (s, 1H), 7.48 (d, | m/e 616 |
| | oxoethyl]-1H-pyrazol-4-yl}- | J = 8.0 Hz, 1H), 7.42-7.28 (m, 3H), 7.13 (t, J = | (M + H)⁺ |
| | 2′,4′-dioxo-2,3-dihydro-3′H- | 8.7 Hz, 2H), 5.24-5.12 (m, 1H), 5.04 (s, | |
| | spiro[indene-1,5′- | 2H), 4.84 (d, J = 17.6 Hz, 1H), 4.69-4.49 (m, | |
| | [1,3]oxazolidin]-3′-yl]-N-(4- | 2H), 4.39 (d, J = 16.8 Hz, 1H), 3.25-3.00 (m, | |
| | fluorobenzyl)-N-[(2S)-1,1,1- | 2H), 3.04 (s, 6H), 2.75-2.65 (m, 1H), 2.57- | |
| | trifluoropropan-2- | 2.49 (m, 1H), 1.38 (d, J = 7.0 Hz, 3H) | |
| | yl]acetamide | | |
| 434 | 2-{(1R)-5-[1-(2-amino-2- | ¹H NMR (400 MHz, DMSO-d₆, 120° C.) δ ppm | (ESI(+)) |
| | oxoethyl)-1H-pyrazol-4-yl]- | 8.08 (s, 1H), 7.83 (s, 1H), 7.55 (s, 1H), 7.48 (d, | m/e 588 |
| | 2′,4′-dioxo-2,3-dihydro-3′H- | J = 8.0 Hz, 1H), 7.42-7.29 (m, 3H), 7.13 (t, J = | (M + H)⁺ |
| | spiro[indene-1,5′- | 8.8 Hz, 2H), 6.89 (brs, 2H), 5.27-5.11 (m, | |
| | [1,3]oxazolidin]-3′-yl}-N-(4- | 1H), 4.84 (d, J = 17.6 Hz, 1H), 4.75 (s, 2H), | |
| | fluorobenzyl)-N-[(2S)-1,1,1- | 4.63 (d, J = 18.4 Hz, 2H), 4.39 (d, J = 16.8 Hz, | |
| | trifluoropropan-2- | 1H), 3.28-3.14 (m, 1H), 3.14-3.00 (m, 1H), | |
| | yl]acetamide | 2.77-2.65 (m, 1H), 2.56-2.48 (m, 1H), 1.38 | |
| | | (d, J = 7.0 Hz, 3H) | |
| 435 | 2-{(1R)-5-[6- | ¹H NMR (400 MHz, DMSO-d₆/D₂O, | (APCI⁺) |
| | (dimethylamino)pyridin-2- | Temp = 90° C.) δ: 8.01 (s, 1H), 7.95 (dd, J = 8.0, | m/z 585 |
| | yl]-2′,4′-dioxo-2,3-dihydro- | 1.6 Hz, 1H), 7.67-7.56 (m, 1H), 7.49 (d, J = | (M + H)⁺ |
| | 3′H-spiro[indene-1,5′- | 8.2 Hz, 1H), 7.41-7.31 (m, 2H), 7.20-7.10 | |
| | [1,3]oxazolidin]-3′-yl}-N-(4- | (m, 3H), 6.66 (d, J = 8.5 Hz, 1H), 5.47-5.03 | |
| | fluorobenzyl)-N-[(2S)-1,1,1- | (m, 1H), 4.94-4.82 (m, 1H), 4.78-4.27 (m, | |
| | trifluoropropan-2- | 3H), 3.25-3.21 (m, 1H), 3.20-3.12 (m, 1H), | |
| | yl]acetamide | 3.12-3.07 (m, 6H), 2.80-2.68 (m, 1H), 2.63- | |
| | | 2.53 (m, 1H), 1.38 (d, J = 7.0 Hz, 3H) | |
| 436 | N-(4-fluorobenzyl)-2-[(1R)- | ¹H NMR (400 MHz, DMSO-d₆/D₂O, | (APCI⁺) |
| | 5-(4-methylpyrimidin-5-yl)- | Temp = 90° C.) δ: 9.01 (s, 1H), 8.55 (s, 1H), 7.56 | m/z 557 |
| | 2′,4′-dioxo-2,3-dihydro-3′H- | (d, J = 8.0 Hz, 1H), 7.49-7.46 (m, 1H), 7.39 | (M + H)⁺ |
| | spiro[indene-1,5′- | (dd, J = 7.9, 1.6 Hz, 1H), 7.37-7.28 (m, 2H), | |
| | [1,3]oxazolidin]-3′-yl]-N- | 7.23-7.10 (m, 2H), 5.37-5.05 (m, 1H), 4.91- | |
| | [(2S)-1,1,1-trifluoropropan- | 4.82 (m, 1H), 4.78-4.29 (m, 3H), 3.34- | |
| | 2-yl]acetamide | 3.27 (m, 1H), 3.21-3.11 (m, 1H), 2.81-2.72 | |
| | | (m, 1H), 2.63-2.55 (m, 1H), 2.45 (s, 3H), | |
| | | 1.38 (d, J = 7.1 Hz, 3H) | |
| 437 | 2-{(1R)-5-[5- | ¹H NMR (400 MHz, DMSO-d₆/D₂O, | (APCI⁺) |
| | (difluoromethyl)pyridin-3- | Temp = 90° C.) δ: 9.05-9.00 (m, 1H), 8.80- | m/z 592 |
| | yl]-2′,4′-dioxo-2,3-dihydro- | 8.76 (m, 1H), 8.27-8.18 (m, 1H), 7.79 (s, | (M + H)⁺ |
| | 3′H-spiro[indene-1,5′- | 1H), 7.73-7.67 (m, 1H), 7.58 (d, J = 8.2 Hz, | |
| | [1,3]oxazolidin]-3′-yl}-N-(4- | 1H), 7.38-7.28 (m, 2H), 7.20-7.14 (m, 2H), | |
| | fluorobenzyl)-N-[(2S)-1,1,1- | 7.14 (s, 1H), 5.33-5.12 (m, 1H), 4.90-4.79 | |
| | trifluoropropan-2- | (m, 1H), 4.75-4.30 (m, 3H), 3.36-3.28 (m, | |
| | yl]acetamide | 1H), 3.23-3.12 (m, 1H), 2.84-2.71 (m, 1H), | |
| | | 2.64-2.55 (m, 1H), 1.38 (d, J = 7.0 Hz, 3H) | |
| 438 | N-(4-fluorobenzyl)-2-[(1R)- | ¹H NMR (400 MHz, DMSO-d₆/D₂O, | (APCI⁺) |
| | 5-(6-methoxypyridin-3-yl)- | Temp = 90° C.) δ: 8.45 (d, J = 2.5 Hz, 1H), 7.99 | m/z 572 |
| | 2′,4′-dioxo-2,3-dihydro-3′H- | (dd, J = 8.6, 2.6 Hz, 1H), 7.65 (s, 1H), 7.61- | (M + H)⁺ |
| | spiro[indene-1,5′- | 7.55 (m, 1H), 7.52-7.48 (m, 1H), 7.39-7.27 | |
| | [1,3]oxazolidin]-3′-yl]-N- | (m, 2H), 7.23-7.10 (m, 2H), 6.90 (d, J = 8.6 | |
| | [(2S)-1,1,1-trifluoropropan- | Hz, 1H), 5.30-5.12 (m, 1H), 4.91-4.79 (m, | |
| | 2-yl]acetamide | 1H), 4.78-4.32 (m, 3H), 3.92 (s, 3H), 3.25- | |
| | | 3.22 (m, 1H), 3.19-3.10 (m, 1H), 2.78-2.67 | |
| | | (m, 1H), 2.62-2.54 (m, 1H), 1.38 (d, J = 7.0 | |
| | | Hz, 3H) | |
| 439 | N-(4-fluorobenzyl)-2-{(1R)- | ¹H NMR (400 MHz, DMSO-d₆/D₂O, | (APCI⁺) |
| | 5-[1-(2-methylpropyl)-1H- | Temp = 90° C.) δ: 8.08 (s, 1H), 7.84 (s, 1H), | m/z 587 |
| | pyrazol-4-yl]-2′,4′-dioxo- | 7.58 (s, 1H), 7.53-7.49 (m, 1H), 7.42-7.37 | (M + H)⁺ |
| | 2,3-dihydro-3′H- | (m, 1H), 7.37-7.28 (m, 2H), 7.20-7.11 (m, | |
| | spiro[indene-1,5′- | 2H), 5.31-5.09 (m, 1H), 4.84 (d, J = 17.7 Hz, | |
| | [1,3]oxazolidin]-3′-yl}-N- | 1H), 4.73-4.30 (m, 3H), 3.94 (d, J = 7.1 Hz, | |
| | [(2S)-1,1,1-trifluoropropan- | 2H), 3.23-3.13 (m, 1H), 3.14-3.04 (m, 1H), | |
| | 2-yl]acetamide | 2.76-2.64 (m, 1H), 2.57-2.54 (m, 1H), 2.22- | |
| | | 2.10 (m, 1H), 1.38 (d, J = 7.1 Hz, 3H), 0.88 | |
| | | (d, J = 6.7 Hz, 6H) | |
| 440 | 2-{(1R)-5-[4- | ¹H NMR (400 MHz, DMSO-d₆/D₂O, | (APCI⁺) |
| | (difluoromethyl)pyridin-2- | Temp = 90° C.) δ: 8.84 (d, J = 5.0 Hz, 1H), 8.16- | m/z 592 |
| | yl]-2′,4′-dioxo-2,3-dihydro- | 8.01 (m, 3H), 7.61-7.49 (m, 2H), 7.38-7.28 | (M + H)⁺ |
| | 3′H-spiro[indene-1,5′- | (m, 2H), 7.21-7.09 (m, 2H), 7.06 (s, 1H), | |
| | [1,3]oxazolidin]-3′-yl}-N-(4- | 5.43-5.01 (m, 1H), 4.96-4.83 (m, 1H), 4.81- | |
| | fluorobenzyl)-N-[(2S)-1,1,1- | 4.20 (m, 3H), 3.35-3.29 (m, 1H), 3.21- | |
| | trifluoropropan-2- | 3.14 (m, 1H), 2.82-2.71 (m, 1H), 2.64-2.55 | |
| | yl]acetamide | (m, 1H), 1.39 (d, J = 7.0 Hz, 3H) | |
| 441 | N-(4-fluorobenzyl)-2-[(1R)- | ¹H NMR (400 MHz, DMSO-d₆/D₂O, | (APCI⁺) |
| | 5-(3-fluoropyridin-2-yl)- | Temp = 90° C.) δ: 8.56-8.52 (m, 1H), 7.91 (s, | m/z 560 |
| | 2′,4′-dioxo-2,3-dihydro-3′H- | 1H), 7.84 (d, J = 8.1 Hz, 1H), 7.81-7.73 (m, | (M + H)⁺ |
| | spiro[indene-1,5′- | 1H), 7.56 (d, J = 8.1 Hz, 1H), 7.52-7.45 (m, | |
| | [1,3]oxazolidin]-3′-yl]-N- | 1H), 7.38-7.29 (m, 2H), 7.20-7.10 (m, 2H), | |
| | [(2S)-1,1,1-trifluoropropan- | 5.26-5.11 (m, 1H), 4.90-4.80 (m, 1H), 4.79- | |
| | 2-yl]acetamide | 4.24 (m, 3H), 3.35-3.29 (m, 1H), 3.22- | |
| | | 3.09 (m, 1H), 2.83-2.70 (m, 1H), 2.67-2.55 | |
| | | (m, 1H), 1.38 (d, J = 7.0 Hz, 3H) | |
| 442 | 2-[(1R)-2′,4′-dioxo-5-(1,3- | ¹H NMR (400 MHz, DMSO-d₆/D₂O, | (APCI⁺) |
| | thiazol-4-yl)-2,3-dihydro- | Temp = 90° C.) δ: 9.13 (d, J = 1.9 Hz, 1H), 8.09 | m/z 547 |
| | 3′H-spiro[indene-1,5′- | (d, J = 1.9 Hz, 1H), 8.00 (s, 1H), 7.94-7.90 | (M + H)⁺ |
| | [1,3]oxazolidin]-3′-yl]-N-(4- | (m, 1H), 7.49 (d, J = 8.1 Hz, 1H), 7.42-7.28 | |
| | fluorobenzyl)-N-[(2S)-1,1,1- | (m, 2H), 7.22-7.11 (m, 2H), 5.40-5.02 (m, | |
| | trifluoropropan-2- | 1H), 4.93-4.79 (m, 1H), 4.78-4.27 (m, 3H), | |
| | yl]acetamide | 3.31-3.28 (m, 1H), 3.19-3.07 (m, 1H), 2.83- | |
| | | 2.67 (m, 1H), 2.65-2.55 (m, 1H), 1.38 (d, J = | |
| | | 7.0 Hz, 3H) | |
| 443 | 4-[(1R)-3′-(2-{(4- | ¹H NMR (400 MHz, DMSO-d₆/D₂O, | (APCI⁺) |
| | fluorobenzyl)[(2S)-1,1,1- | Temp = 90° C.) δ: 7.96-7.85 (m, 2H), 7.79- | m/z 598 |
| | trifluoropropan-2-yl]amino}- | 7.70 (m, 3H), 7.66-7.61 (m, 1H), 7.52 (d, J = | (M + H)⁺ |
| | 2-oxoethyl)-2′,4′-dioxo-2,3- | 8.1 Hz, 1H), 7.38-7.27 (m, 2H), 7.16 (t, J = | |
| | dihydrospiro[indene-1,5′- | 8.4 Hz, 2H), 5.45-5.07 (m, 1H), 4.91-4.78 | |
| | [1,3]oxazolidin]-5-yl]-N- | (m, 1H), 4.76-4.23 (m, 3H), 3.24-3.11 (m, | |
| | methylbenzamide | 2H), 2.83 (s, 3H), 2.81-2.69 (m, 1H), 2.65- | |
| | | 2.54 (m, 1H), 1.38 (d, J = 7.0 Hz, 3H) | |
| 444 | 2-{(1R)-5-[2- | ¹H NMR (400 MHz, DMSO-d₆/D₂O, | (APCI⁺) |
| | (acetylamino)phenyl]-2′,4′- | Temp = 90° C.) δ: 7.54-7.43 (m, 2H), 7.42- | m/z 598 |
| | dioxo-2,3-dihydro-3′H- | 7.29 (m, 7H), 7.20-7.09 (m, 2H), 5.32-5.06 | (M + H)⁺ |
| | spiro[indene-1,5′- | (m, 1H), 4.91-4.75 (m, 1H), 4.75-4.24 (m, | |
| | [1,3]oxazolidin]-3′-yl}-N-(4- | 3H), 3.24-3.06 (m, 2H), 2.82-2.66 (m, 1H), | |
| | fluorobenzyl)-N-[(2S)-1,1,1- | 2.64-2.54 (m, 1H), 1.87 (s, 3H), 1.38 (d, J = | |
| | trifluoropropan-2- | 7.0 Hz, 3H) | |
| | yl]acetamide | | |
| 445 | 2-[(1R)-5-(5-cyanopyridin- | ¹H NMR (400 MHz, DMSO-d₆/D₂O, | (APCI⁺) |
| | 3-yl)-2′,4′-dioxo-2,3- | Temp = 90° C.) δ: 9.13 (d, J = 2.3 Hz, 1H), 8.95 | m/z 567 |
| | dihydro-3′H-spiro[indene- | (d, J = 1.9 Hz, 1H), 8.53 (t, J = 2.1 Hz, 1H), | (M + H)⁺ |
| | 1,5′-[1,3]oxazolidin]-3′-yl]- | 7.81 (s, 1H), 7.75-7.71 (m, 1H), 7.58 (d, J = | |
| | N-(4-fluorobenzyl)-N-[(2S)- | 8.0 Hz, 1H), 7.39-7.29 (m, 2H), 7.23-7.11 | |
| | 1,1,1-trifluoropropan-2- | (m, 2H), 5.41-4.79 (m, 2H), 4.77-4.28 (m, | |
| | yl]acetamide | 3H), 3.24-3.10 (m, 2H), 2.83-2.72 (m, 1H), | |
| | | 2.65-2.55 (m, 1H), 1.38 (d, J = 7.0 Hz, 3H) | |
| 446 | N-(4-fluorobenzyl)-2-[(1R)- | ¹H NMR (400 MHz, DMSO-d₆/D₂O, | (APCI⁺) |
| | 5-(1-methyl-1H-1,2,4- | Temp = 90° C.) δ: 7.95 (s, 1H), 7.78 (s, 1H), | m/z 546 |
| | triazol-5-yl)-2′,4′-dioxo-2,3- | 7.72-7.68 (m, 1H), 7.64-7.57 (m, 1H), 7.39- | (M + H)⁺ |
| | dihydro-3′H-spiro[indene- | 7.29 (m, 2H), 7.23-7.11 (m, 2H), 5.39- | |
| | 1,5′-[1,3]oxazolidin]-3′-yl]- | 4.77 (m, 2H), 4.74-4.30 (m, 3H), 3.95 (s, | |
| | N-[(2S)-1,1,1- | 3H), 3.23-3.08 (m, 2H), 2.86-2.69 (m, 1H), | |
| | trifluoropropan-2- | 2.68-2.57 (m, 1H), 1.38 (d, J = 7.1 Hz, 3H) | |
| | yl]acetamide | | |
| 447 | N-(4-fluorobenzyl)-2-[(1R)- | ¹H NMR (400 MHz, DMSO-d₆/D₂O, | (APCI⁺) |
| | 5-(5-methoxypyridin-3-yl)- | Temp = 90° C.) δ: 8.47 (d, J = 1.9 Hz, 1H), 8.30 | m/z 572 |
| | 2′,4′-dioxo-2,3-dihydro-3′H- | (d, J = 2.8 Hz, 1H), 7.74 (d, J = 1.6 Hz, 1H), | (M + H)⁺ |
| | spiro[indene-1,5′- | 7.70-7.60 (m, 2H), 7.58-7.51 (m, 1H), 7.41- | |
| | [1,3]oxazolidin]-3′-yl]-N- | 7.29 (m, 2H), 7.16 (t, J = 8.7 Hz, 2H), 5.40- | |
| | [(2S)-1,1,1-trifluoropropan- | 5.04 (m, 1H), 4.91-4.78 (m, 1H), 4.74-4.27 | |
| | 2-yl]acetamide | (m, 3H), 3.92 (s, 3H), 3.34-3.26 (m, 1H), | |
| | | 3.21-3.12 (m, 1H), 2.83-2.64 (m, 1H), 2.63- | |
| | | 2.54 (m, 1H), 1.39 (d, J = 7.0 Hz, 3H) | |
| 448 | 2-[(1R)-5-(6-cyanopyridin- | ¹H NMR (400 MHz, DMSO-d₆/D₂O, | (APCI⁺) |
| | 2-yl)-2′,4′-dioxo-2,3- | Temp = 90° C.) δ: 8.27-8.22 (m, 1H), 8.16- | m/z 567 |
| | dihydro-3′H-spiro[indene- | 8.08 (m, 2H), 8.04-7.98 (m, 1H), 7.94-7.90 | (M + H)⁺ |
| | 1,5′-[1,3]oxazolidin]-3′-yl]- | (m, 1H), 7.58 (d, J = 8.1 Hz, 1H), 7.40-7.28 | |
| | N-(4-fluorobenzyl)-N-[(2S)- | (m, 2H), 7.21-7.10 (m, 2H), 5.46-4.80 (m, | |
| | 1,1,1-trifluoropropan-2- | 2H), 4.73-4.32 (m, 3H), 3.24-3.14 (m, 2H), | |
| | yl]acetamide | 2.81-2.70 (m, 1H), 2.66-2.55 (m, 1H), 1.38 | |
| | | (d, J = 7.1 Hz, 3H) | |
| 449 | N-(4-fluorobenzyl)-2-[(1R)- | ¹H NMR (400 MHz, DMSO-d₆/D₂O, | (APCI⁺) |
| | 5-(5-fluoro-3-methylpyridin- | Temp = 90° C.) δ: 7.89 (t, J = 7.5 Hz, 1H), 7.79- | m/z 582 |
| | 2-yl)-2′,4′-dioxo-2,3- | 7.62 (m, 4H), 7.55 (d, J = 8.1 Hz, 1H), 7.45- | (M + H)⁺ |
| | dihydro-3′H-spiro[indene- | 7.32 (m, 2H), 7.13 (t, J = 8.7 Hz, 2H), 5.30- | |
| | 1,5′-[1,3]oxazolidin]-3′-yl]- | 5.05 (m, 1H), 4.90-4.76 (m, 1H), 4.63 (d, J = | |
| | N-[(2S)-1,1,1- | 18.2 Hz, 1H), 4.46-4.33 (m, 1H), 3.37-3.09 | |
| | trifluoropropan-2- | (m, 2H), 2.88-2.69 (m, 1H), 2.66-2.53 (m, | |
| | yl]acetamide | 1H), 1.39 (d, J = 7.1 Hz, 3H) | |
| 450 | N-(4-fluorobenzyl)-2-[(1R)- | ¹H NMR (400 MHz, DMSO-d₆/D₂O, | (APCI⁺) |
| | 5-(5-methyl-1,3,4- | Temp = 90° C.) δ: 7.95 (s, 1H), 7.87 (d, J = 8.1 | m/z 563 |
| | thiadiazol-2-yl)-2′,4′-dioxo- | Hz, 1H), 7.58 (d, J = 8.0 Hz, 1H), 7.38-7.28 | (M + H)⁺ |
| | 2,3-dihydro-3′H- | (m, 2H), 7.23-7.06 (m, 2H), 5.38-4.12 (m, | |
| | spiro[indene-1,5′- | 5H), 3.23-3.10 (m, 2H), 2.78 (s, 3H), 2.75- | |
| | [1,3]oxazolidin]-3′-yl]-N- | 2.53 (m, 2H), 1.38 (d, J = 7.0 Hz, 3H) | |
| | [(2S)-1,1,1-trifluoropropan- | | |
| | 2-yl]acetamide | | |
| 451 | N-(4-fluorobenzyl)-2-[(1R)- | ¹H NMR (400 MHz, DMSO-d₆/D₂O, | (APCI⁺) |
| | 5-(4-methoxypyridin-3-yl)- | Temp = 90° C.)δ: 8.61 (d, J = 6.4 Hz, 1H), 8.52 | m/z 572 |
| | 2′,4′-dioxo-2,3-dihydro-3′H- | (s, 1H), 7.59-7.45 (m, 4H), 7.41-7.23 (m, | (M + H)⁺ |
| | spiro[indene-1,5′- | 2H), 7.22-7.11 (m, 2H), 5.51-4.32 (m, 5H), | |
| | [1,3]oxazolidin]-3′-yl]-N- | 4.01 (s, 3H), 3.23-3.07 (m, 2H), 2.83-2.54 | |
| | [(2S)-1,1,1-trifluoropropan- | (m, 2H), 1.38 (d, J = 7.0 Hz, 3H) | |
| | 2-yl]acetamide | | |
| 452 | N-(4-fluorobenzyl)-2-[(1R)- | ¹H NMR (400 MHz, DMSO-d₆/D₂O, | (APCI⁺) |
| | 5-(6-methoxypyridin-2-yl)- | Temp = 90° C.) δ: 8.07 (s, 1H), 8.00 (dd, J = 8.1, | m/z 572 |
| | 2′,4′-dioxo-2,3-dihydro-3′H- | 1.6 Hz, 1H), 7.81-7.75 (m, 1H), 7.54-7.51 | (M + H)⁺ |
| | spiro[indene-1,5′- | (m, 2H), 7.39-7.29 (m, 2H), 7.22-7.10 (m, | |
| | [1,3]oxazolidin]-3′-yl]-N- | 2H), 6.79 (d, J = 8.0 Hz, 1H), 5.36-4.26 (m, | |
| | [(2S)-1,1,1-trifluoropropan- | 5H), 3.97 (s, 3H), 3.35-3.27 (m, 1H), 3.20- | |
| | 2-yl]acetamide | 3.08 (m, 1H), 2.81-2.70 (m, 1H), 2.65-2.55 | |
| | | (m, 1H), 1.38 (d, J = 7.1 Hz, 3H) | |
| 453 | 2-{(1R)-5-[2- | ¹H NMR (400 MHz, DMSO-d₆/D₂O, | (APCI⁺) |
| | (dimethylamino)pyrimidin- | Temp = 90° C.) δ: 8.64 (s, 2H), 7.62 (s, 1H), 7.58- | m/z 586 |
| | 5-yl]-2′,4′-dioxo-2,3- | 7.53 (m, 1H), 7.51-7.45 (m, 1H), 7.39- | (M + H)⁺ |
| | dihydro-3′H-spiro[indene- | 7.28 (m, 2H), 7.21-7.11 (m, 2H), 5.36-4.95 | |
| | 1,5′-[1,3]oxazolidin]-3′-yl}- | (m, 1H), 4.92-4.23 (m, 4H), 3.24-3.19 (m, | |
| | N-(4-fluorobenzyl)-N-[(2S)- | 1H), 3.18 (s, 6H), 3.16-3.10 (m, 1H), 2.80- | |
| | 1,1,1-trifluoropropan-2- | 2.68 (m, 1H), 2.63-2.54 (m, 1H), 1.38 (d, J = | |
| | yl]acetamide | 7.1 Hz, 3H) | |
| 454 | 2-{(1R)-5-[6- | ¹H NMR (400 MHz, DMSO-d₆/D₂O, | (APCI⁺) |
| | (acetylamino)pyridin-3-yl]- | Temp = 90° C.) δ: 8.59 (d, J = 2.3 Hz, 1H), 8.13- | m/z 599 |
| | 2′,4′-dioxo-2,3-dihydro-3′H- | 8.01 (m, 2H), 7.72-7.65 (m, 1H), 7.64- | (M + H)⁺ |
| | spiro[indene-1,5′- | 7.59 (m, 1H), 7.52 (d, J = 8.0 Hz, 1H), 7.41- | |
| | [1,3]oxazolidin]-3′-yl}-N-(4- | 7.26 (m, 2H), 7.16 (t, J = 8.9 Hz, 2H), 5.40- | |
| | fluorobenzyl)-N-[(2S)-1,1,1- | 4.94 (m, 1H), 4.94-4.12 (m, 4H), 3.23-3.04 | |
| | trifluoropropan-2- | (m, 2H), 2.80-2.65 (m, 1H), 2.64-2.54 (m, | |
| | yl]acetamide | 1H), 2.14 (s, 3H), 1.38 (d, J = 7.0 Hz, 3H) | |
| 455 | N-(4-fluorobenzyl)-2-[(1R)- | ¹H NMR (400 MHz, DMSO-d₆/D₂O, | (APCI⁺) |
| | 5-(1-methyl-1H-1,2,4- | Temp = 90° C.)δ: 8.43 (s, 1H), 8.01 (s, 1H), 7.94 | m/z 546 |
| | triazol-3-yl)-2′,4′-dioxo-2,3- | (d, J = 8.3 Hz, 1H), 7.49 (d, J = 8.0 Hz, 1H), | (M + H)⁺ |
| | dihydro-3′H-spiro[indene- | 7.37-7.29 (m, 2H), 7.19-7.10 (m, 2H), 5.38- | |
| | 1,5′-[1,3]oxazolidin]-3′-yl]- | 4.20 (m, 5H), 3.93 (s, 3H), 3.21-3.07 (m, | |
| | N-[(2S)-1,1,1- | 2H), 2.81-2.56 (m, 2H), 1.38 (d, J = 7.1 Hz, | |
| | trifluoropropan-2- | 3H) | |
| | yl]acetamide | | |
| 456 | N-(4-fluorobenzyl)-2-[(1R)- | ¹H NMR (400 MHz, DMSO-d₆/D₂O, | (APCI⁺) |
| | 5-(1-methyl-1H-pyrazol-4- | Temp = 90° C.) δ: 8.05 (s, 1H), 7.81 (s, 1H), 7.56 | m/z 545 |
| | yl)-2′,4′-dioxo-2,3-dihydro- | (s, 1H), 7.51-7.46 (m, 1H), 7.39 (d, J = 8.0 | (M + H)⁺ |
| | 3′H-spiro[indene-1,5′- | Hz, 1H), 7.37-7.29 (m, 2H), 7.23-7.09 (m, | |
| | [1,3]oxazolidin]-3′-yl]-N- | 2H), 5.40-4.13 (m, 5H), 3.87 (s, 3H), 3.23- | |
| | [(2S)-1,1,1-trifluoropropan- | 3.04 (m, 2H), 2.81-2.53 (m, 2H), 1.38 (d, J = | |
| | 2-yl]acetamide | 7.0 Hz, 3H) | |
| 457 | N-cyclopropyl-5-[(1R)-3′-(2- | ¹H NMR (400 MHz, DMSO-d₆/D₂O, | (APCI⁺) |
| | {(4-fluorobenzyl)[(2S)- | Temp = 90° C.) δ: 7.89 (s, 1H), 7.82 (dd, J = 8.1, | m/z 614 |
| | 1,1,1-trifluoropropan-2- | 1.6 Hz, 1H), 7.51 (d, J = 8.1 Hz, 1H), 7.41- | (M + H)⁺ |
| | yl]amino}-2-oxoethyl)-2′,4′- | 7.27 (m, 2H), 7.21-7.10 (m, 3H), 7.05 (d, J = | |
| | dioxo-2,3- | 3.6 Hz, 1H), 5.49-4.94 (m, 1H), 4.91-4.27 | |
| | dihydrospiro[indene-1,5′- | (m, 4H), 3.24-3.02 (m, 2H), 2.91-2.65 (m, | |
| | [1,3]oxazolidin]-5-yl]furan- | 2H), 2.66-2.53 (m, 1H), 1.38 (d, J = 7.1 Hz, | |
| | 2-carboxamide | 3H), 0.82-0.71 (m, 2H), 0.68-0.58 (m, 2H) | |
| 458 | N-(4-fluorobenzyl)-2-[(1R)- | ¹H NMR (400 MHz, DMSO-d₆/D₂O, | (APCI⁺) |
| | 5-(5-fluoropyridin-2-yl)- | Temp = 90° C.)δ: 8.61 (d, J = 2.9 Hz, 1H), 8.03 | m/z 560 |
| | 2′,4′-dioxo-2,3-dihydro-3′H- | (s, 1H), 8.00 (dd, J = 8.8, 4.4 Hz, 1H), 7.97- | (M + H)⁺ |
| | spiro[indene-1,5′- | 7.92 (m, 1H), 7.75 (td, J = 8.7, 3.0 Hz, 1H), | |
| | [1,3]oxazolidin]-3′-yl]-N- | 7.52 (d, J = 8.0 Hz, 1H), 7.34 (s, 2H), 7.16 (s, | |
| | [(2S)-1,1,1-trifluoropropan- | 2H), 5.41-4.21 (m, 5H), 3.23-3.06 (m, 2H), | |
| | 2-yl]acetamide | 2.82-2.56 (m, 2H), 1.38 (d, J = 7.1 Hz, 3H) | |
| 459 | 4-fluoro-3-[(1R)-3′-(2-{(4- | ¹H NMR (400 MHz, DMSO-d₆/D₂O, | (APCI⁺) |
| | fluorobenzyl)[(2S)-1,1,1- | Temp = 90° C.) δ: 8.02 (dd, J = 7.5, 2.3 Hz, 1H), | m/z 602 |
| | trifluoropropan-2-yl]amino}- | 7.96-7.88 (m, 1H), 7.61 (s, 1H), 7.54 (d, J = | (M + H)⁺ |
| | 2-oxoethyl)-2′,4′-dioxo-2,3- | 1.9 Hz, 2H), 7.40-7.29 (m, 3H), 7.22-7.07 | |
| | dihydrospiro[indene-1,5′- | (m, 2H), 5.47-4.13 (m, 5H), 3.24-3.11 (m, | |
| | [1,3]oxazolidin]-5- | 2H), 2.90-2.56 (m, 2H), 1.38 (d, J = 7.0 Hz, | |
| | yl]benzamide | 3H) | |
| 460 | 5-[(1R)-3′-(2-{(4- | ¹H NMR (400 MHz, DMSO-d₆/D₂O, | (APCI⁺) |
| | fluorobenzyl)[(2S)-1,1,1- | Temp = 90° C.) δ: 8.90 (d, J = 2.2 Hz, 1H), 8.24 | m/z 599 |
| | trifluoropropan-2-yl]amino}- | (dd, J = 8.2, 2.4 Hz, 1H), 8.11 (d, J = 8.2 Hz, | (M + H)⁺ |
| | 2-oxoethyl)-2′,4′-dioxo-2,3- | 1H), 7.82-7.76 (m, 1H), 7.75-7.68 (m, 1H), | |
| | dihydrospiro[indene-1,5′- | 7.58 (d, J = 8.0 Hz, 1H), 7.47-7.29 (m, 2H), | |
| | [1,3]oxazolidin]-5-yl]-N- | 7.16 (t, J = 8.7 Hz, 2H), 5.28-4.97 (m, 1H), | |
| | methylpyridine-2- | 4.93-4.30 (m, 4H), 3.35-3.28 (m, 1H), 3.23- | |
| | carboxamide | 3.14 (m, 1H), 2.89 (s, 3H), 2.83-2.69 (m, | |
| | | 1H), 2.65-2.55 (m, 1H), 1.38 (d, J = 7.0 Hz, | |
| | | 3H) | |
| 461 | 6-[(1R)-3′-(2-{(4- | ¹H NMR (400 MHz, DMSO-d₆/D₂O, | (APCI⁺) |
| | fluorobenzyl)[(2S)-1,1,1- | Temp = 90° C.) δ: 9.09 (d, J = 2.2 Hz, 1H), 8.29 | m/z 585 |
| | trifluoropropan-2-yl]amino}- | (dd, J = 8.3, 2.4 Hz, 1H), 8.13 (s, 1H), 8.07- | (M + H)⁺ |
| | 2-oxoethyl)-2′,4′-dioxo-2,3- | 8.01 (m, 2H), 7.56 (d, J = 8.0 Hz, 1H), 7.39- | |
| | dihydrospiro[indene-1,5′- | 7.28 (m, 2H), 7.22-7.09 (m, 2H), 5.47-4.23 | |
| | [1,3]oxazolidin]-5- | (m, 5H), 3.21-3.11 (m, 2H), 2.84-2.70 (m, | |
| | yl]pyridine-3-carboxamide | 1H), 2.66-2.54 (m, 1H), 1.39 (d, J = 7.1 Hz, | |
| | | 3H) | |
| 462 | N-(4-fluorobenzyl)-2-[(1R)- | ¹H NMR (400 MHz, DMSO-d₆/D₂O, | (APCI⁺) |
| | 5-(2-methoxypyrimidin-5- | Temp = 90° C.) δ: 8.87 (s, 2H), 7.72 (s, 1H), | m/z 573 |
| | yl)-2′,4′-dioxo-2,3-dihydro- | 7.66-7.61 (m, 1H), 7.54 (d, J = 8.0 Hz, 1H), | (M + H)⁺ |
| | 3′H-spiro[indene-1,5′- | 7.39-7.28 (m, 2H), 7.16 (t, J = 8.8 Hz, 2H), | |
| | [1,3]oxazolidin]-3′-yl]-N- | 5.52-4.93 (m, 1H), 4.93-4.27 (m, 4H), 4.00 | |
| | [(2S)-1,1,1-trifluoropropan- | (s, 3H), 3.23-3.03 (m, 2H), 2.84-2.66 (m, | |
| | 2-yl]acetamide | 1H), 2.65-2.54 (m, 1H), 1.38 (d, J = 7.0 Hz, | |
| | | 3H) | |
| 463 | 6-[(1R)-3′-(2-{(4- | ¹H NMR (400 MHz, DMSO-d₆/D₂O, | (APCI⁺) |
| | fluorobenzyl)[(2S)-1,1,1- | Temp = 90° C.) δ: 8.23 (s, 1H), 8.19-8.11 (m, | m/z 585 |
| | trifluoropropan-2-yl]amino}- | 2H), 8.07 (t, J = 7.6 Hz, 1H), 8.04-8.00 (m, | (M + H)⁺ |
| | 2-oxoethyl)-2′,4′-dioxo-2,3- | 1H), 7.57 (d, J = 8.1 Hz, 1H), 7.38-7.29 (m, | |
| | dihydrospiro[indene-1,5′- | 2H), 7.20-7.10 (m, 2H), 5.39-4.25 (m, 5H), | |
| | [1,3]oxazolidin]-5- | 3.24-3.14 (m, 2H), 2.88-2.75 (m, 1H), 2.68- | |
| | yl]pyridine-2-carboxamide | 2.56 (m, 1H), 1.39 (d, J = 7.0 Hz, 3H) | |
| 464 | 4-[(1R)-3′-(2-{(4- | ¹H NMR (400 MHz, DMSO-d₆/D₂O, | (APCI⁺) |
| | fluorobenzyl)[(2S)-1,1,1- | Temp = 90° C.) δ: 7.90-7.87 (m, 1H), 7.51 (d, | m/z 590 |
| | trifluoropropan-2-yl]amino}- | J = 3.2 Hz, 1H), 7.46-7.29 (m, 5H), 7.16 (t, J = | (M + H)⁺ |
| | 2-oxoethyl)-2′,4′-dioxo-2,3- | 8.5 Hz, 2H), 5.38-4.98 (m, 1H), 4.99-4.27 | |
| | dihydrospiro[indene-1,5′- | (m, 4H), 3.20-3.05 (m, 2H), 2.79-2.67 (m, | |
| | [1,3]oxazolidin]-5- | 1H), 2.62-2.54 (m, 1H), 1.38 (d, J = 7.0 Hz, | |
| | yl]thiophene-3-carboxamide | 3H) | |
| 465 | 2-{(1R)-5-[4- | ¹H NMR (400 MHz, DMSO-d₆/D₂O, | (APCI⁺) |
| | (acetylamino)phenyl]-2′,4′- | Temp = 90° C.) δ: 7.66-7.55 (m, 6H), 7.47 (d, | m/z 598 |
| | dioxo-2,3-dihydro-3′H- | J = 8.0 Hz, 1H), 7.41-7.22 (m, 2H), 7.20- | (M + H)⁺ |
| | spiro[indene-1,5′- | 7.08 (m, 2H), 5.44-5.00 (m, 1H), 5.00-4.24 | |
| | [1,3]oxazolidin]-3′-yl}-N-(4- | (m, 4H), 3.23-3.07 (m, 2H), 2.82-2.68 (m, | |
| | fluorobenzyl)-N-[(2S)-1,1,1- | 1H), 2.64-2.53 (m, 1H), 2.07 (s, 3H), 1.38 (d, | |
| | trifluoropropan-2- | J = 7.0 Hz, 3H) | |
| | yl]acetamide | | |
| 466 | 2-[(1R)-5-(1,3-dimethyl-2,4- | ¹H NMR (400 MHz, DMSO-d₆/D₂O, | (APCI⁺) |
| | dioxo-1,2,3,4- | Temp = 90° C.) δ: 7.88 (s, 1H), 7.60 (s, 1H), 7.51 | m/z 603 |
| | tetrahydropyrimidin-5-yl)- | (dd, J = 7.6, 1.6 Hz, 1H), 7.42 (d, J = 8.0 Hz, | (M + H)⁺ |
| | 2′,4′-dioxo-2,3-dihydro-3′H- | 1H), 7.38-7.27 (m, 2H), 7.15 (t, J = 8.5 Hz, | |
| | spiro[indene-1,5′- | 2H), 5.58-5.00 (m, 1H), 4.95-4.19 (m, 4H), | |
| | [1,3]oxazolidin]-3′-yl]-N-(4- | 3.40 (s, 3H), 3.21-3.04 (m, 2H), 2.77-2.61 | |
| | fluorobenzyl)-N-[(2S)-1,1,1- | (m, 1H), 2.64-2.53 (m, 1H), 1.38 (d, J = 7.0 | |
| | trifluoropropan-2- | Hz, 3H) | |
| | yl]acetamide | | |
| 467 | 2-[(1R)-5-(4-cyanopyridin- | ¹H NMR (400 MHz, DMSO-d₆/D₂O, | (APCI⁺) |
| | 2-yl)-2′,4′-dioxo-2,3- | Temp = 90° C.) δ: 8.90 (d, J = 5.0 Hz, 1H), 8.34 | m/z 567 |
| | dihydro-3′H-spiro[indene- | (d, J = 1.5 Hz, 1H), 8.14 (s, 1H), 8.05 (dd, J = | (M + H)⁺ |
| | 1,5′-[1,3]oxazolidin]-3′-yl]- | 8.0, 1.7 Hz, 1H), 7.75 (dd, J = 5.1, 1.4 Hz, 1H), | |
| | N-(4-fluorobenzyl)-N-[(2S)- | 7.57 (d, J = 8.2 Hz, 1H), 7.45-7.27 (m, 2H), | |
| | 1,1,1-trifluoropropan-2- | 7.26-6.99 (m, 2H), 5.21 (s, 1H), 4.90-4.09 | |
| | yl]acetamide | (m, 4H), 3.36-3.26 (m, 1H), 3.23-3.11 (m, | |
| | | 1H), 2.82-2.71 (m, 1H), 2.67-2.56 (m, 1H), | |
| | | 1.38 (d, J = 7.0 Hz, 3H) | |
| 468 | 2-[(1R)-3′-(2-{(4- | ¹H NMR (400 MHz, DMSO-d₆/D₂O, | (APCI⁺) |
| | fluorobenzyl)[(2S)-1,1,1- | Temp = 90° C.) δ: 8.79 (d, J = 5.1 Hz, 1H), 8.27 | m/z 585 |
| | trifluoropropan-2-yl]amino}- | (s, 1H), 8.13 (s, 1H), 8.05 (d, J = 8.3 Hz, 1H), | (M + H)⁺ |
| | 2-oxoethyl)-2′,4′-dioxo-2,3- | 7.73 (dd, J = 5.1, 1.5 Hz, 1H), 7.56 (d, J = 8.1 | |
| | dihydrospiro[indene-1,5′- | Hz, 2H), 7.51-7.48 (m, 1H), 7.38-7.30 (m, | |
| | [1,3]oxazolidin]-5- | 2H), 7.21-7.11 (m, 2H), 5.45-4.23 (m, 5H), | |
| | yl]pyridine-4-carboxamide | 3.21-3.09 (m, 2H), 2.86-2.74 (m, 1H), 2.70- | |
| | | 2.57 (m, 1H), 1.39 (d, J = 7.1 Hz, 3H) | |
| 469 | 2-[(1R)-3′-(2-{(4- | ¹H NMR (400 MHz, DMSO-d₆/D₂O, | (APCI⁺) |
| | fluorobenzyl)[(2S)-1,1,1- | Temp = 90° C.) δ: 7.50-7.46 (m, 2H), 7.43 (dd, | m/z 584 |
| | trifluoropropan-2-yl]amino}- | J = 5.2, 3.0 Hz, 3H), 7.36 (dd, J = 8.3, 3.6 Hz, | (M + H)⁺ |
| | 2-oxoethyl)-2′,4′-dioxo-2,3- | 4H), 7.16 (s, 2H), 4.85 (d, J = 17.5 Hz, 5H), | |
| | dihydrospiro[indene-1,5′- | 3.20 (d, J = 7.7 Hz, 2H), 2.81-2.56 (m, 2H), | |
| | [1,3]oxazolidin]-5- | 1.38 (d, J = 7.1 Hz, 3H) | |
| | yl]benzamide | | |
| 470 | 4-[(1R)-3′-(2-{(4- | ¹H NMR (400 MHz, DMSO-d₆/D₂O, | (APCI⁺) |
| | fluorobenzyl)[(2S)-1,1,1- | Temp = 90° C.)δ: 7.99-7.92 (m, 2H), 7.78- | m/z 584 |
| | trifluoropropan-2-yl]amino}- | 7.71 (m, 3H), 7.64 (dd, J = 8.0, 1.7 Hz, 1H), | (M + H)⁺ |
| | 2-oxoethyl)-2′,4′-dioxo-2,3- | 7.53 (d, J = 8.1 Hz, 1H), 7.38-7.28 (m, 2H), | |
| | dihydrospiro[indene-1,5′- | 7.16 (t, J = 8.7 Hz, 2H), 5.39-5.01 (m, 1H), | |
| | [1,3]oxazolidin]-5- | 4.91-4.19 (m, 4H), 3.24-3.09 (m, 2H), 2.84- | |
| | yl]benzamide | 2.66 (m, 1H), 2.64-2.56 (m, 1H), 1.38 (d, J = | |
| | | 7.0 Hz, 3H) | |
| 471 | 2-{(1R)-5-[3- | ¹H NMR (400 MHz, DMSO-d₆/D₂O, | (APCI⁺) |
| | (acetylamino)phenyl]-2′,4′- | Temp = 90° C.)δ: 7.86-7.82 (m, 1H), 7.61 (s, | m/z 598 |
| | dioxo-2,3-dihydro-3′H- | 1H), 7.59-7.48 (m, 3H), 7.41-7.30 (m, 4H), | (M + H)⁺ |
| | spiro[indene-1,5′- | 7.21-7.11 (m, 2H), 5.54-4.01 (m, 5H), 3.23- | |
| | [1,3]oxazolidin]-3′-yl}-N-(4- | 3.09 (m, 2H), 2.81-2.66 (m, 1H), 2.63- | |
| | fluorobenzyl)-N-[(2S)-1,1,1- | 2.55 (m, 1H), 2.07 (s, 3H), 1.38 (d, J = 7.0 Hz, | |
| | trifluoropropan-2- | 3H) | |
| | yl]acetamide | | |
| 472 | 2-[(1R)-5-(6-cyano-5- | ¹H NMR (400 MHz, DMSO-d₆/D₂O, | (APCI⁺) |
| | methoxypyridin-3-yl)-2′,4′- | Temp = 90° C.) δ: 8.60 (d, J = 1.7 Hz, 1H), 7.93 | m/z 597 |
| | dioxo-2,3-dihydro-3′H- | (d, J = 1.8 Hz, 1H), 7.85 (d, J = 1.7 Hz, 1H), | (M + H)⁺ |
| | spiro[indene-1,5′- | 7.77 (dd, J = 8.2, 1.7 Hz, 1H), 7.60 (d, J = 8.0 | |
| | [1,3]oxazolidin]-3′-yl]-N-(4- | Hz, 1H), 7.38-7.29 (m, 2H), 7.21-7.04 (m, | |
| | fluorobenzyl)-N-[(2S)-1,1,1- | 2H), 5.63-4.28 (m, 5H), 4.07 (s, 3H), 3.29 (s, | |
| | trifluoropropan-2- | 1H), 3.22-3.14 (m, 1H), 2.94-2.72 (m, 1H), | |
| | yl]acetamide | 2.68-2.56 (m, 1H), 1.38 (d, J = 7.1 Hz, 3H) | |
| 473 | N-(4-fluorobenzyl)-2-[(1R)- | ¹H NMR (400 MHz, DMSO-d₆/D₂O, | (APCI⁺) |
| | 5-(1-methyl-1H-pyrazol-3- | Temp = 90° C.) δ: 7.78 (s, 1H), 7.72 (dd, J = 7.9, | m/z 545 |
| | yl)-2′,4′-dioxo-2,3-dihydro- | 1.5 Hz, 1H), 7.66 (d, J = 2.3 Hz, 1H), 7.42 (d, J = | (M + H)⁺ |
| | 3′H-spiro[indene-1,5′- | 8.1 Hz, 1H), 7.39-7.28 (m, 2H), 7.21-7.07 | |
| | [1,3]oxazolidin]-3′-yl]-N- | (m, 2H), 6.67 (d, J = 2.3 Hz, 1H), 5.40-4.87 | |
| | [(2S)-1,1,1-trifluoropropan- | (m, 1H), 4.93-4.23 (m, 4H), 3.88 (s, 3H), | |
| | 2-yl]acetamide | 3.23-3.04 (m, 2H), 2.79-2.68 (m, 1H), 2.64- | |
| | | 2.53 (m, 1H), 1.38 (d, J = 7.0 Hz, 3H) | |
| 474 | 2-[(1R)-2′,4′-dioxo-5-(1,3,5- | ¹H NMR (400 MHz, DMSO-d₆/D₂O, | (APCI⁺) |
| | trimethyl-1H-pyrazol-4-yl)- | Temp = 90° C.)δ: 7.45 (d, J = 7.9 Hz, 1H), 7.40- | m/z 573 |
| | 2,3-dihydro-3′H- | 7.30 (m, 2H), 7.27 (s, 1H), 7.23-7.10 (m, | (M + H)⁺ |
| | spiro[indene-1,5′- | 3H), 5.42-4.30 (m, 5H), 3.69 (s, 3H), 3.23- | |
| | [1,3]oxazolidin]-3′-yl]-N-(4- | 3.05 (m, 2H), 2.76-2.54 (m, 2H), 2.21 (s, | |
| | fluorobenzyl)-N-[(2S)-1,1,1- | 3H), 2.12 (s, 3H), 1.38 (d, J = 7.0 Hz, 3H) | |
| | trifluoropropan-2- | | |
| | yl]acetamide | | |
| 475 | 2-[(1R)-5-(3,4- | ¹H NMR (400 MHz, DMSO-d₆/D₂O, | (APCI⁺) |
| | difluorophenyl)-2′,4′-dioxo- | Temp = 90° C.) δ: 7.73-7.63 (m, 2H), 7.62- | m/z 577 |
| | 2,3-dihydro-3′H- | 7.57 (m, 1H), 7.55-7.41 (m, 3H), 7.39-7.29 | (M + H)⁺ |
| | spiro[indene-1,5′- | (m, 2H), 7.23-7.10 (m, 2H), 5.48-4.19 (m, | |
| | [1,3]oxazolidin]-3′-yl]-N-(4- | 5H), 3.20-3.07 (m, 2H), 2.87-2.54 (m, 2H), | |
| | fluorobenzyl)-N-[(2S)-1,1,1- | 1.38 (d, J = 7.1 Hz, 3H) | |
| | trifluoropropan-2- | | |
| | yl]acetamide | | |
| 476 | 2-[(1R)-5-(2,5-difluoro-4- | ¹H NMR (400 MHz, DMSO-d₆/D₂O, | (APCI⁺) |
| | methoxyphenyl)-2′,4′-dioxo- | Temp = 90° C.) δ: 7.73-7.63 (m, 2H), 7.62- | m/z 607 |
| | 2,3-dihydro-3′H- | 7.57 (m, 1H), 7.55-7.41 (m, 3H), 7.39-7.29 | (M + H)⁺ |
| | spiro[indene-1,5′- | (m, 2H), 7.23-7.10 (m, 2H), 5.48-4.19 (m, | |
| | [1,3]oxazolidin]-3′-yl]-N-(4- | 5H), 3.20-3.07 (m, 2H), 2.87-2.54 (m, 2H), | |
| | fluorobenzyl)-N-[(2S)-1,1,1- | 1.38 (d, J = 7.1 Hz, 3H) | |
| | trifluoropropan-2- | | |
| | yl]acetamide | | |
| 477 | 2-[(1R)-2′,4′-dioxo-5- | ¹H NMR (400 MHz, DMSO-d₆/D₂O, | (APCI⁺) |
| | (pyrimidin-5-yl)-2,3- | Temp = 90° C.)δ: 9.17 (s, 1H), 9.09 (s, 2H), 7.80 | m/z 543 |
| | dihydro-3′H-spiro[indene- | (d, J = 1.6 Hz, 1H), 7.71 (dd, J = 8.0, 1.7 Hz, | (M + H)⁺ |
| | 1,5′-[1,3]oxazolidin]-3′-yl]- | 1H), 7.59 (d, J = 8.0 Hz, 1H), 7.41-7.28 (m, | |
| | N-(4-fluorobenzyl)-N-[(2S)- | 2H), 7.16 (t, J = 8.6 Hz, 2H), 5.40-4.96 (m, | |
| | 1,1,1-trifluoropropan-2- | 1H), 4.97-4.29 (m, 4H), 3.35-3.27 (m, 1H), | |
| | yl]acetamide | 3.23-3.11 (m, 1H), 2.85-2.55 (m, 2H), 1.39 | |
| | | (d, J = 7.0 Hz, 3H) | |
| 478 | 2-{(1R)-2′,4′-dioxo-5-[5- | ¹H NMR (400 MHz, DMSO-d₆/D₂O, | (APCI⁺) |
| | (trifluoromethyl)pyridin-3- | Temp = 90° C.)δ: 9.15 (d, J = 2.1 Hz, 1H), 8.99- | m/z 610 |
| | yl]-2,3-dihydro-3′H- | 8.90 (m, 1H), 8.42-8.35 (m, 1H), 7.83 (d, J = | (M + H)⁺ |
| | spiro[indene-1,5′- | 1.5 Hz, 1H), 7.74 (dd, J = 8.0, 1.8 Hz, 1H), | |
| | [1,3]oxazolidin]-3′-yl}-N-(4- | 7.59 (d, J = 8.0 Hz, 1H), 7.42-7.27 (m, 2H), | |
| | fluorobenzyl)-N-[(2S)-1,1,1- | 7.16 (t, J = 8.7 Hz, 2H), 5.44-4.99 (m, 1H), | |
| | trifluoropropan-2- | 4.94-4.25 (m, 4H), 3.24-3.15 (m, 2H), 2.85- | |
| | yl]acetamide | 2.70 (m, 1H), 2.68-2.55 (m, 1H), 1.39 (d, J = | |
| | | 7.1 Hz, 3H) | |
| 479 | 2-[(1R)-2′,4′-dioxo-5-(1,3- | ¹H NMR (400 MHz, DMSO-d₆/D₂O, | (APCI⁺) |
| | thiazol-2-yl)-2,3-dihydro- | Temp = 90° C.) δ: 7.97 (s, 1H), 7.94-7.88 (m, | m/z 548 |
| | 3′H-spiro[indene-1,5′- | 2H), 7.77-7.71 (m, 1H), 7.54 (d, J = 8.1 Hz, | (M + H)⁺ |
| | [1,3]oxazolidin]-3′-yl]-N-(4- | 1H), 7.41-7.28 (m, 2H), 7.20-7.11 (m, 2H), | |
| | fluorobenzyl)-N-[(2S)-1,1,1- | 5.55-4.17 (m, 5H), 3.23-3.12 (m, 2H), 2.84- | |
| | trifluoropropan-2- | 2.56 (m, 2H), 1.38 (d, J = 7.1 Hz, 3H) | |
| | yl]acetamide | | |
| 480 | 2-[(1R)-5-(2-cyanopyridin- | ¹H NMR (400 MHz, DMSO-d₆/D₂O, | (APCI⁺) |
| | 4-yl)-2′,4′-dioxo-2,3- | Temp = 90° C.) δ: 8.79 (d, J = 5.3 Hz, 1H), 8.29 | m/z 567 |
| | dihydro-3′H-spiro[indene- | (d, J = 2.3 Hz, 1H), 8.01 (dd, J = 5.2, 1.9 Hz, | (M + H)⁺ |
| | 1,5′-[1,3]oxazolidin]-3′-yl]- | 1H), 7.89 (d, J = 1.6 Hz, 1H), 7.79 (dd, J = 8.0, | |
| | N-(4-fluorobenzyl)-N-[(2S)- | 1.7 Hz, 1H), 7.60 (d, J = 8.1 Hz, 1H), 7.44- | |
| | 1,1,1-trifluoropropan-2- | 7.27 (m, 2H), 7.23-7.07 (m, 2H), 5.43-4.95 | |
| | yl]acetamide | (m, 1H), 4.93-4.30 (m, 4H), 3.24-3.14 (m, | |
| | | 2H), 2.81-2.54 (m, 2H), 1.38 (d, J = 7.1 Hz, | |
| | | 3H) | |
| 481 | 2-[(1R)-2′,4′-dioxo-5- | ¹H NMR (400 MHz, DMSO-d₆/D₂O, | (APCI⁺) |
| | (pyrazin-2-yl)-2,3-dihydro- | Temp = 90° C.) δ: 9.18 (d, J = 1.5 Hz, 1H), 8.72- | m/z 543 |
| | 3′H-spiro[indene-1,5′- | 8.69 (m, 1H), 8.61 (d, J = 2.5 Hz, 1H), 8.12 (s, | (M + H)⁺ |
| | [1,3]oxazolidin]-3′-yl]-N-(4- | 1H), 8.04 (d, J = 8.4 Hz, 1H), 7.58 (d, J = 8.0 | |
| | fluorobenzyl)-N-[(2S)-1,1,1- | Hz, 1H), 7.41-7.26 (m, 2H), 7.23-7.07 (m, | |
| | trifluoropropan-2- | 2H), 5.45-4.19 (m, 5H), 3.25-3.13 (m, 2H), | |
| | yl]acetamide | 2.84-2.53 (m, 2H), 1.38 (d, J = 7.1 Hz, 3H) | |
| 482 | 2-{(1R)-5-[4- | ¹H NMR (400 MHz, DMSO-d₆/D₂O, | (APCI⁺) |
| | (carbamoylamino)phenyl]- | Temp = 90° C.) δ: 7.64-7.59 (m, 1H), 7.58- | m/z 599 |
| | 2′,4′-dioxo-2,3-dihydro-3′H- | 7.53 (m, 3H), 7.51-7.44 (m, 3H), 7.40-7.29 | (M + H)⁺ |
| | spiro[indene-1,5′- | (m, 2H), 7.22-7.11 (m, 2H), 5.44-4.24 (m, | |
| | [1,3]oxazolidin]-3′-yl}-N-(4- | 5H), 3.24-3.01 (m, 2H), 2.85-2.54 (m, 2H), | |
| | fluorobenzyl)-N-[(2S)-1,1,1- | 1.38 (d, J = 7.1 Hz, 3H) | |
| | trifluoropropan-2- | | |
| | yl]acetamide | | |
| 483 | 2-[(1R)-5-(2-cyanopyridin- | ¹H NMR (400 MHz, DMSO-d₆/D₂O, | (APCI⁺) |
| | 3-yl)-2′,4′-dioxo-2,3- | Temp = 90° C.) δ: 8.76 (dd, J = 4.8, 1.6 Hz, 1H), | m/z 567 |
| | dihydro-3′H-spiro[indene- | 8.08 (dd, J = 8.0, 1.5 Hz, 1H), 7.85-7.75 (m, | (M + H)⁺ |
| | 1,5′-[1,3]oxazolidin]-3′-yl]- | 1H), 7.68-7.54 (m, 3H), 7.46-7.27 (m, 2H), | |
| | N-(4-fluorobenzyl)-N-[(2S)- | 7.16 (t, J = 8.9 Hz, 2H), 5.48-4.25 (m, 5H), | |
| | 1,1,1-trifluoropropan-2- | 3.23-3.09 (m, 2H), 2.84-2.55 (m, 2H), 1.38 | |
| | yl]acetamide | (d, J = 7.0 Hz, 3H) | |
| 484 | 2-[(1R)-5-(6-cyano-5- | ¹H NMR (400 MHz, DMSO-d₆/D₂O, | (APCI⁺) |
| | fluoropyridin-3-yl)-2′,4′- | Temp = 90° C.) δ: 9.00-8.90 (m, 1H), 8.31 (dd, | m/z 584 |
| | dioxo-2,3-dihydro-3′H- | J = 10.2, 1.8 Hz, 1H), 7.87 (s, 1H), 7.78 (dd, J = | (M + H)⁺ |
| | spiro[indene-1,5′- | 8.1, 1.8 Hz, 1H), 7.61 (d, J = 8.0 Hz, 1H), | |
| | [1,3]oxazolidin]-3′-yl]-N-(4- | 7.40-7.26 (m, 2H), 7.23-7.09 (m, 2H), 5.51- | |
| | fluorobenzyl)-N-[(2S)-1,1,1- | 4.17 (m, 5H), 3.24-3.01 (m, 2H), 2.95- | |
| | trifluoropropan-2- | 2.56 (m, 2H), 1.38 (d, J = 7.0 Hz, 3H) | |
| | yl]acetamide | | |
| 485 | 3-[(1R)-3′-(2-{(4- | ¹H NMR (400 MHz, DMSO-d₆/D₂O, | (APCI⁺) |
| | fluorobenzyl)[(2S)-1,1,1- | Temp = 90° C.) δ: 8.13 (t, J = 1.9 Hz, 1H), 7.87 | m/z 584 |
| | trifluoropropan-2-yl]amino}- | (dt, J = 7.7, 1.4 Hz, 1H), 7.82 (dt, J = 7.9, 1.4 | (M + H)⁺ |
| | 2-oxoethyl)-2′,4′-dioxo-2,3- | Hz, 1H), 7.73 (d, J = 1.6 Hz, 1H), 7.66 (dd, J = | |
| | dihydrospiro[indene-1,5′- | 8.0, 1.7 Hz, 1H), 7.55 (q, J = 7.9 Hz, 2H), 7.34 | |
| | [1,3]oxazolidin]-5- | (s, 2H), 7.16 (t, J = 8.9 Hz, 2H), 5.38-4.02 | |
| | yl]benzamide | (m, 5H), 3.25-3.11 (m, 2H), 2.85-2.55 (m, | |
| | | 2H), 1.39 (d, J = 7.0 Hz, 3H) | |
| 523 | 2-[(1R)-5-(5-cyanothiophen- | ¹H NMR (400 MHz, DMSO-d₆/D₂O, | (APCI⁺) |
| | 2-yl)-2′,4′-dioxo-2,3- | Temp = 120° C.) δ: 7.85 (d, J = 4.0 Hz, 1H), 7.74 | m/z 572 |
| | dihydro-3′H-spiro[indene- | (s, 1H), 7.66 (dd, J = 8.0, 1.8 Hz, 1H), 7.59 (d, | (M + H)⁺ |
| | 1,5′-[1,3]oxazolidin]-3′-yl]- | J = 4.0 Hz, 1H), 7.51 (d, J = 8.1 Hz, 1H), 7.37- | |
| | N-(4-fluorobenzyl)-N-[(2S)- | 7.30 (m, 2H), 7.14 (t, 2H), 5.23-5.09 (m, | |
| | 1,1,1-trifluoropropan-2- | 1H), 4.82 (d, J = 17.6 Hz, 1H), 4.69-4.55 (m, | |
| | yl]acetamide | 2H), 4.46-4.34 (m, 1H), 3.37-3.11 (m, 2H), | |
| | | 2.80-2.69 (m, 1H), 2.63-2.53 (m, 1H), 1.39 | |
| | | (d, J = 7.1 Hz, 3H) | |
| 524 | 2-[(1R)-5-(4-cyano-3- | ¹H NMR (400 MHz, DMSO-d₆/D₂O, | (APCI⁺) |
| | fluorophenyl)-2′,4′-dioxo- | Temp = 120° C.) δ: 7.89 (t, J = 7.5 Hz, 1H), 7.77- | m/z 582 |
| | 2,3-dihydro-3′H- | 7.65 (m, 4H), 7.55 (d, J = 8.1 Hz, 1H), 7.38- | (M + H)⁺ |
| | spiro[indene-1,5′- | 7.30 (m, 2H), 7.13 (t, J = 8.7 Hz, 2H), 5.29- | |
| | [1,3]oxazolidin]-3′-yl]-N-(4- | 5.04 (m, 1H), 4.83 (d, J = 17.6 Hz, 1H), 4.75- | |
| | fluorobenzyl)-N-[(2S)-1,1,1- | 4.33 (m, 3H), 3.34-3.11 (m, 2H), 2.81-2.53 | |
| | trifluoropropan-2- | (m, 2H), 1.39 (d, J = 7.1 Hz, 3H) | |
| | yl]acetamide | | |
| 525 | 2-[(1R)-5-(5-cyanothiophen- | ¹H NMR (400 MHz, DMSO-d₆/D₂O, | (APCI⁺) |
| | 3-yl)-2′,4′-dioxo-2,3- | Temp = 120° C.) δ: 8.25-8.22 (m, 2H), 7.73 (s, | m/z 572 |
| | dihydro-3′H-spiro[indene- | 1H), 7.65 (d, J = 8.1 Hz, 1H), 7.48 (d, J = 8.0 | (M + H)⁺ |
| | 1,5′-[1,3]oxazolidin]-3′-yl]- | Hz, 1H), 7.39-7.29 (m, 2H), 7.13 (t, J = 8.7 | |
| | N-(4-fluorobenzyl)-N-[(2S)- | Hz, 2H), 5.37-5.05 (m, 1H), 4.82 (d, J = 17.7 | |
| | 1,1,1-trifluoropropan-2- | Hz, 1H), 4.75-4.28 (m, 3H), 3.32-3.06 (m, | |
| | yl]acetamide | 2H), 2.80-2.52 (m, 2H), 1.39 (d, J = 7.0 Hz, | |
| | | 3H) | |
| 530 | 2-[(1R)-5-(4,6- | ¹H NMR (400 MHz, DMSO-d₆/D₂O, | (APCI⁺) |
| | difluoropyrimidin-5-yl)- | Temp = 120° C.) δ: 7.78 (s, 1H), 7.69 (d, J = 7.7 | m/z 579 |
| | 2′,4′-dioxo-2,3-dihydro-3′H- | Hz, 1H), 7.39-7.29 (m, 3H), 7.13 (t, J = 8.7 | (M + H)⁺ |
| | spiro[indene-1,5′- | Hz, 2H), 5.26-5.14 (m, 1H), 4.87-4.75 (m, | |
| | [1,3]oxazolidin]-3′-yl]-N-(4- | 1H), 4.73-4.36 (m, 3H), 3.21-3.09 (m, 2H), | |
| | fluorobenzyl)-N-[(2S)-1,1,1- | 2.76-2.52 (m, 2H), 1.38 (d, J = 7.2 Hz, 3H) | |
| | trifluoropropan-2- | | |
| | yl]acetamide | | |
| 531 | 2-{(1R)-5-[2-(acetylamino)- | ¹H NMR (400 MHz, DMSO-d₆/D₂O, | (APCI⁺) |
| | 5-methylpyridin-3-yl]-2′,4′- | Temp = 120° C.) δ: 8.30-8.21 (m, 1H), 7.68- | m/z 613 |
| | dioxo-2,3-dihydro-3′H- | 7.59 (m, 1H), 7.48-7.42 (m, 2H), 7.38-7.31 | (M + H)⁺ |
| | spiro[indene-1,5′- | (m, 3H), 7.14 (t, J = 8.8 Hz, 2H), 5.35-5.11 | |
| | [1,3]oxazolidin]-3′-yl}-N-(4- | (m, 1H), 4.83 (d, J = 17.6 Hz, 1H), 4.73-4.54 | |
| | fluorobenzyl)-N-[(2S)-1,1,1- | (m, 2H), 4.53-4.36 (m, 1H), 3.30-3.12 (m, | |
| | trifluoropropan-2- | 2H), 2.84-2.55 (m, 2H), 2.35 (s, 3H), 1.88 (s, | |
| | yl]acetamide | 3H), 1.39 (d, J = 7.0 Hz, 3H) | |
| 532 | N-(4-fluorobenzyl)-2-[(1R)- | ¹H NMR (400 MHz, DMSO-d₆/D₂O, | (APCI⁺) |
| | 5-(5-fluoropyridin-3-yl)- | Temp = 120° C.) δ: 8.73 (t, J = 1.9 Hz, 1H), | m/z 560 |
| | 2′,4′-dioxo-2,3-dihydro-3′H- | 8.52 (d, J = 2.7 Hz, 1H), 7.95-7.89 (m, 1H), | (M + H)⁺ |
| | spiro[indene-1,5′- | 7.75 (s, 1H), 7.68-7.63 (m, 1H), 7.55 (d, J = | |
| | [1,3]oxazolidin]-3′-yl]-N- | 8.0 Hz, 1H), 7.39-7.30 (m, 2H), 7.14 (t, 2H), | |
| | [(2S)-1,1,1-trifluoropropan- | 5.24-5.12 (m, 1H), 4.83 (d, J = 17.5 Hz, 1H), | |
| | 2-yl]acetamide | 4.70-4.51 (m, 2H), 4.50-4.32 (m, 1H), 3.33- | |
| | | 3.13 (m, 2H), 2.89-2.56 (m, 2H), 1.39 (d, J = | |
| | | 7.1 Hz, 3H) | |
| 533 | N-(4-fluorobenzyl)-2-[(1R)- | | (APCI⁺) |
| | 5-(furan-3-yl)-2′,4′-dioxo- | | m/z 531 |
| | 2,3-dihydro-3′H- | | (M + H)⁺ |
| | spiro[indene-1,5′- | | |
| | [1,3]oxazolidin]-3′-yl]-N- | | |
| | [(2S)-1,1,1-trifluoropropan- | | |
| | 2-yl]acetamide | | |
| 534 | 2-[(1R)-2′,4′-dioxo-5-(2- | ¹H NMR (400 MHz, DMSO-d₆/D₂O, | (APCI⁺) |
| | oxo-2,3-dihydro-1H- | Temp = 120° C.) δ: 7.78 (s, 1H), 7.69 (d, J = 7.7 | m/z 597 |
| | benzimidazol-5-yl)-2,3- | Hz, 1H), 7.58 (s, 1H), 7.55-7.41 (m, 1H), | (M + H)⁺ |
| | dihydro-3′H-spiro[indene- | 7.39-7.30 (m, 3H), 7.25-7.21 (m, 1H), 7.17- | |
| | 1,5′-[1,3]oxazolidin]-3′-yl]- | 7.09 (m, 2H), 5.25-5.10 (m, 1H), 4.89- | |
| | N-(4-fluorobenzyl)-N-[(2S)- | 4.75 (m, 1H), 4.72-4.53 (m, 2H), 4.50-4.34 | |
| | 1,1,1-trifluoropropan-2- | (m, 1H), 3.28-3.10 (m, 2H), 2.79-2.60 (m, | |
| | yl]acetamide | 2H), 1.38 (dd, J = 7.1, 2.8 Hz, 3H) | |
| 535 | N-(4-fluorobenzyl)-2-[(1R)- | ¹H NMR (400 MHz, DMSO-d₆/D₂O, | (APCI⁺) |
| | 5-(4-methylpyrimidin-2-yl)- | Temp = 120° C.) δ: 8.70 (d, J = 5.1 Hz, 1H), 8.37 | m/z 557 |
| | 2′,4′-dioxo-2,3-dihydro-3′H- | (s, 1H), 8.31 (d, J = 8.2 Hz, 1H), 7.52 (d, J = | (M + H)⁺ |
| | spiro[indene-1,5′- | 8.1 Hz, 1H), 7.43-7.27 (m, 3H), 7.13 (t, J = | |
| | [1,3]oxazolidin]-3′-yl]-N- | 8.6 Hz, 2H), 5.27-5.08 (m, 1H), 4.90-4.75 | |
| | [(2S)-1,1,1-trifluoropropan- | (m, 1H), 4.68-4.55 (m, 2H), 4.47-4.33 (m, | |
| | 2-yl]acetamide | 1H), 3.34-3.15 (m, 2H), 2.84-2.60 (m, 2H), | |
| | | 2.55 (s, 3H), 1.39 (dd, J = 7.0, 3.5 Hz, 3H) | |
| 536 | 2-{(1R)-2′,4′-dioxo-5-[1- | ¹H NMR (400 MHz, DMSO-d₆/D₂O, | (APCI⁺) |
| | (tetrahydro-2H-pyran-2-yl)- | Temp = 120° C.) δ: 7.83-7.78 (m, 2H), 7.73 | m/z 615 |
| | 1H-pyrazol-3-yl]-2,3- | (d, J = 8.1 Hz, 1H), 7.43 (d, J = 8.0 Hz, 1H), | (M + H)⁺ |
| | dihydro-3′H-spiro[indene- | 7.37-7.30 (m, 2H), 7.13 (t, J = 8.7 Hz, 2H), | |
| | 1,5′-[1,3]oxazolidin]-3′-yl}- | 6.72 (d, J = 2.5 Hz, 1H), 5.45 (dd, J = 9.2, 2.7 | |
| | N-(4-fluorobenzyl)-N-[(2S)- | Hz, 1H), 5.23-5.11 (m, 1H), 4.90-4.77 (m, | |
| | 1,1,1-trifluoropropan-2- | 1H), 4.72-4.55 (m, 2H), 4.45-4.36 (m, 1H), | |
| | yl]acetamide | 3.99-3.89 (m, 1H), 3.72-3.60 (m, 1H), 3.28- | |
| | | 3.09 (m, 2H), 2.79-2.53 (m, 2H), 2.18- | |
| | | 2.06 (m, 1H), 2.06-1.96 (m, 2H), 1.78-1.67 | |
| | | (m, 1H), 1.63-1.53 (m, 2H), 1.39 (d, J = 7.1 | |
| | | Hz, 3H) | |
| 537 | N-(4-fluorobenzyl)-2-[(1R)- | ¹H NMR (400 MHz, DMSO-d₆/D₂O, | (APCI⁺) |
| | 5-(5-fluoro-6-methylpyridin- | Temp = 120° C.) δ: 7.99 (s, 1H), 7.92 (d, J = 8.1 | m/z 574 |
| | 2-yl)-2′,4′-dioxo-2,3- | Hz, 1H), 7.79-7.74 (m, 1H), 7.60 (t, J = 8.8 | (M + H)⁺ |
| | dihydro-3′H-spiro[indene- | Hz, 1H), 7.50 (d, J = 8.1 Hz, 1H), 7.37-7.31 | |
| | 1,5′-[1,3]oxazolidin]-3′-yl]- | (m, 2H), 7.14 (t, J = 8.6 Hz, 2H), 5.30-4.28 | |
| | N-[(2S)-1,1,1- | (m, 5H), 3.35-3.16 (m, 2H), 2.80-2.53 (m, | |
| | trifluoropropan-2- | 2H), 2.52 (s, 3H), 1.39 (d, J = 7.1 Hz, 3H) | |
| | yl]acetamide | | |
| 538 | 2-{(1R)-5-[6- | ¹H NMR (400 MHz, DMSO-d₆/D₂O, | (APCI⁺) |
| | (difluoromethyl)pyridin-3- | Temp = 120° C.) δ: 8.94 (d, J = 2.2 Hz, 1H), | m/z 592 |
| | yl]-2′,4′-dioxo-2,3-dihydro- | 8.23 (dd, J = 8.2, 2.3 Hz, 1H), 7.80-7.73 (m, | (M + H)⁺ |
| | 3′H-spiro[indene-1,5′- | 2H), 7.68 (dd, J = 7.9, 1.7 Hz, 1H), 7.56 (d, J = | |
| | [1,3]oxazolidin]-3′-yl}-N-(4- | 8.1 Hz, 1H), 7.39-7.30 (m, 2H), 7.18-7.09 | |
| | fluorobenzyl)-N-[(2S)-1,1,1- | (m, 2H), 6.88 (t, J = 55.1 Hz, 1H), 5.33-4.91 | |
| | trifluoropropan-2- | (m, 1H), 4.83 (d, J = 17.6 Hz, 1H), 4.75-4.58 | |
| | yl]acetamide | (m, 2H), 4.52-4.30 (m, 1H), 3.36-3.12 (m, | |
| | | 2H), 2.86-2.54 (m, 2H), 1.39 (d, J = 7.0 Hz, | |
| | | 3H) | |
| 539 | 2-{(1R)-5-[6- | ¹H NMR (400 MHz, DMSO-d₆/D₂O, | (APCI⁺) |
| | (difluoromethyl)pyridin-2- | Temp = 120° C.) δ: 8.10-8.06 (m, 3H), 8.01 | m/z 592 |
| | yl]-2′,4′-dioxo-2,3-dihydro- | (d, J = 8.2 Hz, 1H), 7.67-7.62 (m, 1H), 7.55 | (M + H)⁺ |
| | 3′H-spiro[indene-1,5′- | (d, J = 8.1 Hz, 1H), 7.38-7.29 (m, 2H), 7.14 | |
| | [1,3]oxazolidin]-3′-yl}-N-(4- | (t, J = 8.7 Hz, 2H), 6.89 (t, J = 55.1 Hz, 1H), | |
| | fluorobenzyl)-N-[(2S)-1,1,1- | 5.24-5.12 (m, 1H), 4.93-4.74 (m, 1H), 4.68- | |
| | trifluoropropan-2- | 4.57 (m, 1H), 4.47-4.36 (m, 2H), 3.37- | |
| | yl]acetamide | 3.15 (m, 2H), 2.84-2.61 (m, 2H), 1.39 (d, J = | |
| | | 7.1 Hz, 3H) | |
| 540 | N-(4-fluorobenzyl)-2-[(1R)- | ¹H NMR (400 MHz, DMSO-d₆/D₂O, | (APCI⁺) |
| | 5-(furan-2-yl)-2′,4′-dioxo- | Temp = 120° C.) δ: 7.70-7.59 (m, 2H), 7.44 | m/z 531 |
| | 2,3-dihydro-3′H- | (d, J = 8.0 Hz, 1H), 7.39-7.29 (m, 2H), 7.13 | (M + H)⁺ |
| | spiro[indene-1,5′- | (t, J = 8.6 Hz, 2H), 6.90 (d, J = 3.4 Hz, 1H), | |
| | [1,3]oxazolidin]-3′-yl]-N- | 6.83-6.71 (m, 1H), 6.61-6.55 (m, 1H), 5.27- | |
| | [(2S)-1,1,1-trifluoropropan- | 4.32 (m, 5H), 3.24-3.11 (m, 2H) 2.78-2.59 | |
| | 2-yl]acetamide | (m, 2H), 1.39 (d, J = 7.2 Hz, 3H) | |
| 541 | 2-{(1R)-5-[1- | ¹H NMR (400 MHz, DMSO-d₆/D₂O, | (APCI⁺) |
| | (ethoxymethyl)-1H- | Temp = 120° C.) δ: 7.75 (s, 1H), 7.70-7.65 (m, | m/z 589 |
| | imidazol-2-yl]-2′,4′-dioxo- | 1H), 7.58-7.50 (m, 1H), 7.47 (s, 1H), 7.38- | (M + H)⁺ |
| | 2,3-dihydro-3′H- | 7.27 (m, 2H), 7.20-7.10 (m, 3H), 5.38 (s, | |
| | spiro[indene-1,5′- | 2H), 5.24-4.38 (m, 5H), 3.54 (q, J = 7.1 Hz, | |
| | [1,3]oxazolidin]-3′-yl}-N-(4- | 2H), 3.11-3.31 (m, 2H), 2.81-2.54 (m, 2H), | |
| | fluorobenzyl)-N-[(2S)-1,1,1- | 1.39 (d, J = 7.0 Hz, 3H), 1.10 (t, J = 6.9 Hz, | |
| | trifluoropropan-2- | 3H) | |
| | yl]acetamide | | |
| 542 | 2-[(1R)-5-(5-cyano-1,2- | ¹H NMR (400 MHz, DMSO-d₆/D₂O, | (APCI⁺) |
| | dimethyl-6-oxo-1,6- | Temp = 120° C.) δ: 7.86 (s, 1H), 7.48 (d, J = | m/z 611 |
| | dihydropyridin-3-yl)-2′,4′- | 7.9 Hz, 1H), 7.36-7.31 (m, 3H), 7.25 (d, J = | (M + H)⁺ |
| | dioxo-2,3-dihydro-3′H- | 7.9 Hz, 1H), 7.13 (t, J = 8.8 Hz, 2H), 5.24- | |
| | spiro[indene-1,5′- | 5.07 (m, 1H), 4.89-4.78 (m, 1H), 4.70-4.55 | |
| | [1,3]oxazolidin]-3′-yl]-N-(4- | (m, 2H), 4.51-4.37 (m, 1H), 3.59 (s, 3H), | |
| | fluorobenzyl)-N-[(2S)-1,1,1- | 3.31-3.12 (m, 2H), 2.83-2.56 (m, 2H), 2.37 | |
| | trifluoropropan-2- | (s, 3H), 1.39 (d, J = 7.0 Hz, 3H) | |
| | yl]acetamide | | |
| 543 | 3-[(1R)-3′-(2-{(4- | ¹H NMR (400 MHz, DMSO-d₆/D₂O, | (APCI⁺) |
| | fluorobenzyl)[(2S)-1,1,1- | Temp = 120° C.) δ: 7.63 (d, J = 4.9 Hz, 1H), | m/z 590 |
| | trifluoropropan-2-yl]amino}- | 7.50-7.38 (m, 3H), 7.38-7.27 (m, 2H), 7.18- | (M + H)⁺ |
| | 2-oxoethyl)-2′,4′-dioxo-2,3- | 7.09 (m, 3H), 5.24-5.12 (m, 1H), 4.83 (d, J = | |
| | dihydrospiro[indene-1,5′- | 17.7 Hz, 1H), 4.67-4.51 (m, 2H), 4.48- | |
| | [1,3]oxazolidin]-5- | 4.30 (m, 1H), 3.32-3.09 (m, 2H), 2.85-2.55 | |
| | yl]thiophene-2-carboxamide | (m, 2H), 1.38 (d, J = 7.0 Hz, 3H) | |
| 544 | 2-[(1R)-5-(2,6-dioxo- | | (APCI⁺) |
| | 1,2,5,6-tetrahydropyrimidin- | | m/z 575 |
| | 5-yl)-2′,4′-dioxo-2,3- | | (M + H)⁺ |
| | dihydro-3′H-spiro[indene- | | |
| | 1,5′-[1,3]oxazolidin]-3′-yl]- | | |
| | N-(4-fluorobenzyl)-N-[(2S)- | | |
| | 1,1,1-trifluoropropan-2- | | |
| | yl]acetamide | | |
| 545 | 2-[(1R)-5-(2,4-dimethyl-1,3- | ¹H NMR (400 MHz, DMSO-d₆/D₂O, | (APCI⁺) |
| | thiazol-5-yl)-2′,4′-dioxo-2,3- | Temp = 120° C.) δ: 7.49-7.44 (m, 2H), 7.40- | m/z 576 |
| | dihydro-3′H-spiro[indene- | 7.30 (m, 3H), 7.13 (t, J = 8.7 Hz, 2H), 5.23- | (M + H)⁺ |
| | 1,5′-[1,3]oxazolidin]-3′-yl]- | 5.11 (m, 1H), 4.92-4.77 (m, 1H), 4.67-4.54 | |
| | N-(4-fluorobenzyl)-N-[(2S)- | (m, 2H), 4.46-4.33 (m, 1H), 3.29-3.15 (m, | |
| | 1,1,1-trifluoropropan-2- | 2H), 2.81-2.69 (m, 1H), 2.61 (s, 3H), 2.59- | |
| | yl]acetamide | 2.52 (m, 1H), 2.37 (s, 3H), 1.38 (d, J = 7.1 Hz, | |
| | | 3H) | |
| 546 | 2-{(1R)-5-[1-(cyanomethyl)- | ¹H NMR (400 MHz, DMSO-d₆/D₂O, | (APCI⁺) |
| | 3,5-dimethyl-1H-pyrazol-4- | Temp = 120° C.) δ: 7.46 (d, J = 8.0 Hz, 1H), 7.37- | m/z 598 |
| | yl]-2′,4′-dioxo-2,3-dihydro- | 7.31 (m, 2H), 7.29 (s, 1H), 7.21 (d, J = 7.9 | (M + H)⁺ |
| | 3′H-spiro[indene-1,5′- | Hz, 1H), 7.13 (t, J = 8.6 Hz, 2H), 5.28-4.37 | |
| | [1,3]oxazolidin]-3′-yl}-N-(4- | (m, 5H), 3.28-3.17 (m, 2H), 2.81-2.49 (m, | |
| | fluorobenzyl)-N-[(2S)-1,1,1- | 2H), 2.27 (s, 3H), 2.14 (s, 3H), 1.39 (d, J = 7.1 | |
| | trifluoropropan-2- | Hz, 3H) | |
| | yl]acetamide | | |
| 547 | 2-{(1R)-5-[3- | ¹H NMR (400 MHz, DMSO-d₆/D₂O, | (APCI⁺) |
| | (carbamoylamino)phenyl]- | Temp = 120° C.) δ: 7.70-7.67 (m, 1H), 7.59 (s, | m/z 599 |
| | 2′,4′-dioxo-2,3-dihydro-3′H- | 1H), 7.54-7.50 (m, 1H), 7.49-7.45 (m, 1H), | (M + H)⁺ |
| | spiro[indene-1,5′- | 7.40-7.29 (m, 4H), 7.23-7.18 (m, 1H), 7.14 | |
| | [1,3]oxazolidin]-3′-yl}-N-(4- | (t, J = 8.7 Hz, 2H), 5.28-5.10 (m, 1H), 4.90- | |
| | fluorobenzyl)-N-[(2S)-1,1,1- | 4.75 (m, 1H), 4.69-4.57 (m, 2H), 4.49-4.35 | |
| | trifluoropropan-2- | (m, 1H), 3.28-3.15 (m, 2H), 2.80-2.57 (m, | |
| | yl]acetamide | 2H), 1.39 (d, J = 7.1 Hz, 3H) | |
| 565 | N-(4-fluorobenzyl)-2-{(1R)- | ¹H NMR (400 MHz, DMSO-d₆, 120° C.) δ | (ESI(+)) |
| | 5-[1-(2-hydroxyethyl)-1H- | ppm 8.05 (s, 1H), 7.80 (s, 1H), 7.54 (s, 1H), | m/e 575 |
| | pyrazol-4-yl]-2′,4′-dioxo- | 7.47 (d, J = 8.0 Hz, 1H), 7.40-7.28 (m, 3H), | (M + H)⁺ |
| | 2,3-dihydro-3′H- | 7.13 (t, J = 8.6 Hz, 2H), 5.24-5.11 (m, 1H), | |
| | spiro[indene-1,5′- | 4.90-4.79 (m, 1H), 4.68-4.54 (m, 2H), 4.48- | |
| | [1,3]oxazolidin]-3′-yl}-N- | 4.32 (m, 2H), 4.17 (t, J = 5.7 Hz, 2H), 3.81 | |
| | [(2S)-1,1,1-trifluoropropan- | (q, J = 5.6 Hz, 2H), 3.27-2.99 (m, 2H), 2.76- | |
| | 2-yl]acetamide | 2.64 (m, 1H), 2.58-2.49 (m, 1H), 1.38 (d, J = | |
| | | 7.0 Hz, 3H) | |

### Example 486

### N-(4-fluorobenzyl)-N-[(2S)-1,1,1-trifluoropropan-2-yl]-2-(2,2′,5-trioxo-2′,3′-dihydro-1H,1′H-spiro[imidazolidine-4,4′-quinolin]-1-yl)acetamide

A solution of N-(4-fluorobenzyl)-N-[(2S)-1,1,1-trifluoropropan-2-yl]-2-(2,3′,5-trioxo-2′,3′-dihydro-1H-spiro[imidazolidine-4,1′-inden]-1-yl)acetamide (50 mg, 0.105 mmol) in sulfuric acid (0.7 mL, 13.13 mmol) was treated portion wise with sodium azide (6.81 mg, 0.105 mmol) and then stirred at room temperature for 35 minutes. The mixture was poured into ice water and extracted with ethyl acetate. The organic extracts were washed with water, saturated sodium bicarbonate solution and brine then dried over Na₂SO₄, filtered, and concentrated. Purification by reverse-phase HPLC gave the title compound. ¹H NMR (400 MHz, DMSO-d₆) δ 8.21-6.73 (m, 10H), 5.41-3.54 (m, 6H), 3.17-2.70 (m, 1H), 1.65-1.08 (m, 3H). MS (ESI) m/e 493 (M+H)⁺.

### Example 487

### 2-(2,5-dioxo-1H-spiro[imidazolidine-4,1′-inden]-1-yl)-N-(4-fluorobenzyl)-N-[(2S)-1,1,1-trifluoropropan-2-yl]acetamide

### Example 487A

### spiro[imidazolidine-4,1′-indene]-2,5-dione

A mixture of 3′-hydroxy-2′,3′-dihydrospiro[imidazolidine-4,1′-indene]-2,5-dione (60 mg, 0.275 mmol) and 4-methylbenzenesulfonic acid hydrate (10.46 mg, 0.055 mmol) in toluene was heated to reflux for 18 hours. The solvent was removed under vacuum to give the title compound.

### Example 487B

### 2-(2,5-dioxo-1H-spiro[imidazolidine-4,1′-inden]-1-yl)-N-(4-fluorobenzyl)-N-[(2S)-1,1,1-trifluoropropan-2-yl]acetamide

The title compound was prepared as described in EXAMPLE 281F, substituting spiro[imidazolidine-4,1′-indene]-2,5-dione for (R)-5-bromo-2,3-dihydrospiro[indene-1,5′-oxazolidine]-2′,4′-dione. ¹H NMR (400 MHz, DMSO-d₆) δ 8.81-8.52 (m, 1H), 7.59-6.90 (m, 9H), 6.43-6.21 (m, 1H), 5.52-4.02 (m, 5H), 1.46-1.17 (m, 3H). MS (ESI) m/e 462 (M+H)⁺.

### Example 488

### 2-{5′-[1-(2-amino-2-oxoethyl)-1H-pyrazol-4-yl]-2,3′,5-trioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide

The title compound was prepared as described in EXAMPLE 295, substituting 2-(5′-bromo-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.94 (dd, J=9.3, 2.9 Hz, 1H), 8.33 (d, J=5.7 Hz, 1H), 8.21-7.96 (m, 2H), 7.92 (d, J=1.5 Hz, 1H), 7.74-7.02 (m, 7H), 5.36 (h, J=7.7 Hz, 1H), 5.00-4.04 (m, 6H), 3.23-2.82 (m, 2H), 1.50-1.16 (m, 3H). MS (ESI⁺) m/z 601 (M+H)⁺.

### Example 489

### N-(4-fluorobenzyl)-N-[(2S)-1,1,1-trifluoropropan-2-yl]-2-[2,3′,5-trioxo-5′-(6-oxo-1,6-dihydropyridin-3-yl)-2′,3′-dihydro-1H-spiro[imidazolidine-4,1′-inden]-1-yl]acetamide

The title compound was prepared as described in EXAMPLE 295, substituting 2-(5′-bromo-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide. ¹H NMR (400 MHz, DMSO-d₆) δ 11.91 (s, 1H), 8.97 (d, J=8.2 Hz, 1H), 8.16-7.00 (m, 10H), 6.45 (d, J=9.3 Hz, 1H), 5.37 (p, J=8.0 Hz, 1H), 4.96-4.04 (m, 4H), 3.25-2.86 (m, 2H), 1.54-1.22 (m, 3H). MS (ESI⁺) m/z 571 (M+H)⁺.

### Example 490

### 2-[(1R)-5-{[(4-aminophenyl)carbamoyl]amino}-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

### Example 490A

### tert-butyl (4-(3-((R)-3′-(2-((4-fluorobenzyl)((S)-1,1,1-trifluoropropan-2-yl)amino)-2-oxoethyl)-2′,4′-dioxo-2,3-dihydrospiro[indene-1,5′-oxazolidin]-5-yl)ureido)phenyl)carbamate

The title compound was prepared as described in EXAMPLE 282, substituting 2-((R)-5-amino-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and tert-butyl (4-aminophenyl)carbamate for 3,3-difluoroazetidine hydrochloride.

### Example 490B

### 2-[(1R)-5-{[(4-aminophenyl)carbamoyl]amino}-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 302, substituting tert-butyl (4-(3-((R)-3′-(2-((4-fluorobenzyl)((S)-1,1,1-trifluoropropan-2-yl)amino)-2-oxoethyl)-2′,4′-dioxo-2,3-dihydrospiro[indene-1,5′-oxazolidin]-5-yl)ureido)phenyl)carbamate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 9.03 (d, J=8.2 Hz, 1H), 8.96 (d, J=6.1 Hz, 1H), 7.62-7.55 (m, 1H), 7.51-7.42 (m, 2H), 7.41-7.18 (m, 5H), 7.18 7.06 (m, 3H), 5.39 (m, 1H), 5.11-5.00 (m, 1H), 4.93 (q, J=18.7, 17.5 Hz, 1H), 4.84-4.69 (m, 1H), 4.55 (t, J=16.3 Hz, 2H), 4.20 (d, J=17.1 Hz, 1H), 3.13 (dt, J=15.2, 7.6 Hz, 1H), 3.00 (ddd, J=16.3, 8.7, 4.0 Hz, 1H), 2.63 (ddd, J=15.1, 8.4, 6.6 Hz, 1H), 2.28 (s, 1H), 1.40-1.19 (m, 3H). MS ESI⁺ 714 (M+H)⁺.

### Example 491

### N-[(1S)-1-cyclopropylethyl]-2-[(4R)-2,5-dioxo-2′,3′-dihydro-1H-spiro[imidazolidine-4,1′-inden]-1-yl]-N-(4-fluorobenzyl)acetamide

### Example 491A

### (R)-2′,3′-dihydrospiro[imidazolidine-4,1′-indene]-2,5-dione and (S)-2′,3′-dihydrospiro[imidazolidine-4,1′-indene]-2,5-dione

The title compounds were prepared as described in EXAMPLE 290B, substituting 2,3-dihydro-1H-inden-1-one for 6-bromobenzo[b]thiophen-3 (2H)-one 1,1-dioxide followed by chiral SFC to give (R)-2′,3′-Dihydrospiro[imidazolidine-4,1′-indene]-2,5-dione (eluted first) and (S)-2′,3′-dihydrospiro[imidazolidine-4,1′-indene]-2,5-dione (eluted second); single crystal x-ray diffraction determined the absolute configuration of each.

### Example 491B

### N-[(1S)-1-cyclopropylethyl]-2-[(4R)-2,5-dioxo-2′,3′-dihydro-H-spiro[imidazolidine-4,1′-inden]-1-yl]-N-(4-fluorobenzyl)acetamide

The title compound was prepared as described in EXAMPLE 281F, substituting (R)-2′,3′-dihydrospiro[imidazolidine-4,1′-indene]-2,5-dione for (R)-5-bromo-2,3-dihydrospiro[indene-1,5′-oxazolidine]-2′,4′-dione and (S)-2-bromo-N-(1-cyclopropylethyl)-N-(4-fluorobenzyl)acetamide for (S)-2-bromo-N-(4-fluorobenzyl)-N-(1,1,1-trifluoropropan-2-yl)acetamide. ¹H NMR (400 MHz, DMSO-d₆, Temp=90° C.) δ ppm 8.46 (s, 1H), 7.48-6.97 (m, 8H), 4.64 (s, 2H), 3.04 (t, J=7.2 Hz, 3H), 2.56 (dt, J=13.4, 6.7 Hz, 1H), 2.22 (dt, J=13.2, 7.7 Hz, 1H), 1.38-1.06 (m, 5H), 1.04-0.80 (m, 2H), 0.49 (dp, J=8.4, 4.3 Hz, 1H), 0.37-0.15 (m, 2H); MS (ESI(+)) m/e 436.0 (M+H)⁺.

### Example 492

### N-[(1S)-1-cyclopropylethyl]-2-[(4S)-2,5-dioxo-2′,3′-dihydro-1H-spiro[imidazolidine-4,1′-inden]-1-yl]-N-(4-fluorobenzyl)acetamide

The title compound was prepared as described in EXAMPLE 281F, substituting (S)-2′,3′-dihydrospiro[imidazolidine-4,1′-indene]-2,5-dione for (R)-5-bromo-2,3-dihydrospiro[indene-1,5′-oxazolidine]-2′,4′-dione and (S)-2-bromo-N-(1-cyclopropylethyl)-N-(4-fluorobenzyl)acetamide for (S)-2-bromo-N-(4-fluorobenzyl)-N-(1,1,1-trifluoropropan-2-yl)acetamide. ¹H NMR (400 MHz, DMSO-d₆, Temp=90° C.) δ ppm 8.46 (s, 1H), 7.48-6.97 (m, 8H), 4.64 (s, 2H), 3.04 (t, J=7.2 Hz, 3H), 2.56 (dt, J=13.4, 6.7 Hz, 1H), 2.22 (dt, J=13.2, 7.7 Hz, 1H), 1.38-1.06 (m, 5H), 1.04-0.80 (m, 2H), 0.49 (dp, J=8.4, 4.3 Hz, 1H), 0.37-0.15 (m, 2H); MS (ESI(+)) m/e 436.0 (M+H)⁺.

### Example 493

### 2-(7′-bromo-1′-methyl-2,5-dioxo-2′,3′-dihydro-1H,1′H-spiro[imidazolidine-4,4′-quinolin]-1-yl)-N-[(1S)-1-cyclopropylethyl]-N-(4-fluorobenzyl)acetamide

The title compound was prepared as described in EXAMPLE 348, substituting 7-bromo-2,3-dihydroquinolin-4(1H)-one for 2,3-dihydroquinolin-4(1H)-one in EXAMPLE 348A and substituting (S)-2-bromo-N-(1-cyclopropylethyl)-N-(4-fluorobenzyl)acetamide for (S)-2-bromo-N-(4-fluorobenzyl)-N-(1,1,1-trifluoropropan-2-yl)acetamide in EXAMPLE 348C. ¹H NMR (500 MHz, DMSO-d₆) δ 8.85 (d, J=23.7 Hz, 1H), 7.44 (dd, J=8.5, 5.5 Hz, 1H), 7.27 (dd, J=8.5, 5.6 Hz, 1H), 7.24-7.15 (m, 1H), 7.15-7.02 (m, 2H), 6.82 (t, J=2.3 Hz, 1H), 6.75 (ddd, J=8.3, 6.2, 1.8 Hz, 1H), 4.72 (s, 1H), 4.62-4.55 (m, 1H), 4.35 (ddd, J=71.1, 16.6, 7.4 Hz, 1H), 4.23-4.06 (m, 1H), 3.49 (ddd, J=11.9, 7.8, 4.1 Hz, 1H), 3.27 (dd, J=10.6, 7.3 Hz, 1H), 2.95-2.84 (m, 4H), 2.73 (s, 1H), 2.20-2.09 (m, 1H), 2.08-1.93 (m, 1H), 1.19 (dd, J=6.5, 3.0 Hz, 1H), 1.10 (d, J=6.8 Hz, 2H), 0.56-0.42 (m, 1H), 0.40-0.06 (m, 3H). MS ESI⁺ 545 (M+H)⁺.

### Example 494

### N-(4-fluorobenzyl)-2-{6-[(methylcarbamoyl)amino]-2′,4′-dioxo-3,4-dihydro-2H,3′H-spiro[naphthalene-1,5′-[1,3]oxazolidin]-3′-yl}-N-[(2S)-1, 1,1-trifluoropropan-2-yl]acetamide

### Example 494A

### 6-bromo-3,4-dihydronaphthalen-1(2H)-one

To a solution of 6-amino-3,4-dihydronaphthalen-1(2H)-one (5 g, 31 mmol in 25% HBr (10 mL) at 0° C. was added a solution of NaNO₂ (2.78 g, 40.3 mmol) in water (10 mL) slowly. The mixture was added to a solution of CuBr (4.45 g, 31 mmol) in 40% HBr (10 mL) and the mixture was stirred for 1.5 hours while warming to room temperature. The mixture was extracted with ethyl acetate, dried over MgSO₄, and filtered. The solvent was removed under vacuum. The crude material was purified by silica gel column chromatography to afford the title compound.

### Example 494B

### 6-bromo-3,4-dihydro-2H-spiro[naphthalene-1,5′-oxazolidine]-2′,4′-dione

The title compound was prepared as described in EXAMPLE 301A-D, substituting 6-bromo-3,4-dihydronaphthalen-1(2H)-one for 7-bromochroman-4-one in EXAMPLE 301A.

### Example 494C

### 2-(6-bromo-2′,4′-dioxo-3,4-dihydro-2H-spiro[naphthalene-1,5′-oxazolidin]-3′-yl)-N-(4-fluorobenzyl)-N—((S)-1,1,1-trifluoropropan-2-yl)acetamide

The title compound was prepared as described in EXAMPLE 281F, substituting 6-bromo-3,4-dihydro-2H-spiro[naphthalene-1,5′-oxazolidine]-2′,4′-dione for (R)-5-bromo-2,3-dihydrospiro[indene-1,5′-oxazolidine]-2′,4′-dione.

### Example 494D

### 2-(6-amino-2′,4′-dioxo-3,4-dihydro-2H-spiro[naphthalene-1,5′-oxazolidin]-3′-yl)-N-(4-fluorobenzyl)-N—((S)-1,1,1-trifluoropropan-2-yl)acetamide

The title compound was prepared as described in EXAMPLE 284D-E, substituting 2-(6-bromo-2′,4′-dioxo-3,4-dihydro-2H-spiro[naphthalene-1,5′-oxazolidin]-3′-yl)-N-(4-fluorobenzyl)-N—((R)-1,1,1-trifluoropropan-2-yl)acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide.

### Example 494E

### N-(4-fluorobenzyl)-2-{6-[(methylcarbamoyl)amino]-2′,4′-dioxo-3,4-dihydro-2H,3′H-spiro[naphthalene-1,5′-[1,3]oxazolidin]-3′-yl}-N-[(2S)-1, 1,1-trifluoropropan-2-yl]acetamide

The title compound was prepared as described in EXAMPLE 282, substituting 2-(6-amino-2′,4′-dioxo-3,4-dihydro-2H-spiro[naphthalene-1,5′-oxazolidin]-3′-yl)-N-(4-fluorobenzyl)-N—((S)-1,1,1-trifluoropropan-2-yl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, dimethylsulfoxide-d₆) δ ppm 1.33 (dd, J=17.1, 6.9 Hz, 3H), 1.65-2.25 (m, 4H), 2.55-2.64 (m, 3H), 2.65-2.86 (m, 2H), 4.11-4.99 (m, 4H), 5.39 (p, J=7.8 Hz, 1H), 5.96-6.12 (m, 1H), 7.05-7.42 (m, 7H), 8.51-8.66 (m, 1H). MS (ESI⁺) m/z 551.0 (M+H)⁺.

### Example 495

### 2-[5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

### Example 495A

### N-(2,5-dioxo-2′,3′-dihydrospiro[imidazolidine-4,1′-inden]-5′-yl)acetamide

The title compound was prepared as described in EXAMPLE 290B, substituting N-(1-oxo-2,3-dihydro-1H-inden-5-yl)acetamide for 6-bromobenzo[b]thiophen-3 (2H)-one 1,1-dioxide.

### Example 495B

### 2-[5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 281F, substituting N-(2,5-dioxo-2′,3′-dihydrospiro[imidazolidine-4,1′-inden]-5′-yl)acetamide for (R)-5-bromo-2,3-dihydrospiro[indene-1,5′-oxazolidine]-2′,4′-dione. ¹H NMR (501 MHz, DMSO-d₆) δ 9.97 (s, 1H), 9.02-8.11 (m, 1H), 7.83-6.75 (m, 7H), 5.21 (ddt, J=148.3, 13.9, 7.1 Hz, 1H), 4.94-3.99 (m, 4H), 2.99 (q, J=6.9, 6.5 Hz, 2H), 2.60-2.51 (m, 1H), 2.19 (tdd, J=12.8, 10.0, 6.2 Hz, 1H), 2.04 (d, J=2.2 Hz, 3H), 1.32 (dd, J=31.9, 6.9 Hz, 3H). MS (ESI⁺) m/z 521 (M+H)⁺.

### Example 499

### N-(4-fluorobenzyl)-2-(4′-hydroxy-2,5-dioxo-3′,4′-dihydro-1H,2′H-spiro[imidazolidine-4, 1′-naphthalen]-1-yl)-N-[(2S)-1,1,1-trifluoropropan-2-yl]acetamide

### Example 499A

### 4′-hydroxy-3′,4′-dihydro-2′H-spiro[imidazolidine-4,1′-naphthalene]-2,5-dione

The title compound was prepared as described in EXAMPLE 290B, substituting 4-hydroxy-3,4-dihydronaphthalen-1(2H)-one for 6-bromobenzo[b]thiophen-3 (2H)-one 1,1-dioxide.

### Example 499B

### N-(4-fluorobenzyl)-2-(4′-hydroxy-2,5-dioxo-3′,4′-dihydro-1H,2′H-spiro[imidazolidine-4, 1′-naphthalen]-1-yl)-N-[(2S)-1,1,1-trifluoropropan-2-yl]acetamide

The title compound was prepared as described in EXAMPLE 281F, substituting 4′-hydroxy-3′,4′-dihydro-2′H-spiro[imidazolidine-4,1′-naphthalene]-2,5-dione for (R)-5-bromo-2,3-dihydrospiro[indene-1,5′-oxazolidine]-2′,4′-dione. ¹H NMR (400 MHz, DMSO-d₆) δ 9.15-8.71 (m, 1H), 7.63-6.97 (m, 8H), 5.51-3.95 (m, 7H), 2.42-1.68 (m, 4H), 1.52-1.16 (m, 3H). MS (ESI) m/e 494 (M+H)⁺.

### Example 500

### 2-(6-{-[2-(dimethylamino)-2-oxoethyl]-1H-pyrazol-4-yl}-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide

### Example 500A

### (1′-(2-((4-fluorobenzyl)((S)-1,1,1-trifluoropropan-2-yl)amino)-2-oxoethyl)-1,1-dioxido-2′,5′-dioxo-2H-spiro[benzo[b]thiophene-3,4′-imidazolidin]-6-yl)boronic acid

The title compound was prepared as described in EXAMPLE 353A, substituting 2-(6-bromo-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide for 2-((R)-5-bromo-2′,4′-dioxo-2,3-dihydrospiro[indene-1,5′-oxazolidin]-3′-yl)-N—((S)-1-cyclopropylethyl)-N-(4-fluorobenzyl)acetamide.

### Example 500B

### 2-(6-{-[2-(dimethylamino)-2-oxoethyl]-1H-pyrazol-4-yl}-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide

The title compound was prepared as described in EXAMPLE 432B, substituting 2-(4-bromo-1H-pyrazol-1-yl)-N,N-dimethylacetamide for 2-(4-bromo-1H-pyrazol-1-yl)-N-methylacetamide and (1′-(2-((4-fluorobenzyl)((S)-1,1,1-trifluoropropan-2-yl)amino)-2-oxoethyl)-1,1-dioxido-2′,5′-dioxo-2H-spiro[benzo[b]thiophene-3,4′-imidazolidin]-6-yl)boronic acid for 2-((R)-2′,4′-dioxo-5-(4,4,5,5-tetramethyl-1,3,2-dioxaborolan-2-yl)-2,3-dihydrospiro[indene-1,5′-oxazolidin]-3′-yl)-N-(4-fluorobenzyl)-N—((S)-1,1,1-trifluoropropan-2-yl)acetamide. ¹H NMR (400 MHz, DMSO-d₆, 120° C.) δ ppm 8.92 (brs, 1H), 8.22 (s, 1H), 8.02-7.90 (m, 3H), 7.64 (dd, J=8.2, 6.5 Hz, 1H), 7.39-7.27 (m, 2H), 7.18-7.05 (m, 2H), 5.18 (brs, 1H), 5.05 (s, 2H), 4.82 (dd, J=17.6, 5.5 Hz, 1H), 4.66-4.44 (m, 2H), 4.30 (dd, J=16.6, 5.8 Hz, 1H), 3.96 (dd, J=14.4, 3.8 Hz, 1H), 3.79 (dd, J=14.4, 4.3 Hz, 1H), 2.96 (s, 6H), 1.35 (dd, J=7.1, 2.1 Hz, 3H). MS (ESI⁺) m/z 665 (M+H)⁺.

### Example 501

### 2-[5′-(acetylamino)-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

2-[5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide (140 mg, 0.269 mmol) in 3 mL acetone was cooled to −78° C. KMnO₄ (255 mg, 1.614 mmol) and ferric chloride (109 mg, 0.672 mmol) were added. The mixture was stirred at −78° C. for 1 hour and at room temperature overnight. The suspension was filtered and the solid was rinsed thoroughly with acetone. The filtrate was concentrated and purified by silica gel column chromatography to provide the title compound. ¹H NMR (400 MHz, DMSO-d₆) δ 10.33 (s, 1H), 8.91 (d, J=8.5 Hz, 1H), 8.09 (d, J=5.4 Hz, 1H), 7.87-7.02 (m, 6H), 5.21 (dp, J=122.3, 7.7 Hz, 1H), 4.99-3.96 (m, 4H), 3.16-2.77 (m, 2H), 2.05 (d, J=19.0 Hz, 3H), 1.38-1.19 (m, 3H). MS (ESI⁺) m/z 535 (M+H)⁺.

### Example 503

### N²-(4-fluorobenzyl)-N²-({(1R)-5-[(methylcarbamoyl)amino]-2′,4′-dioxo-2,3-dihydro-3′H-spiro[indene-1,5′-[1,3]oxazolidin]-3′-yl}acetyl)-L-alaninamide

### Example 503A

### (S)-2-((4-fluorobenzyl)amino)propanamide

(S)-2-Aminopropanamide (0.3 g, 3.40 mmol) was dissolved in methanol (2.3 ml) and 4-fluorobenzaldehyde (0.845 g, 6.81 mmol) was added. The reaction mixture was stirred at room temperature for 30 minutes. The clear solution was cooled to 0° C. and sodium triacetoxyhydroborate (1.443 g, 6.81 mmol) was added as a solid in a single portion. The cloudy reaction mixture was stirred at room temperature for 24 hours. A solution of 10 mL aqueous 2N NaOH was added and the reaction mixture was allowed to stir at room temperature for 20 minutes. The reaction mixture was poured into a separatory funnel containing additional aqueous 2N NaOH (5 mL) and the aqueous layer was extracted with ethyl acetate. The combined organic layers were washed with brine, dried over anhydrous sodium sulfate, filtered, and concentrated. The residue was purified by silica gel column chromatography to give the title compound.

### Example 503B

### N²-(4-fluorobenzyl)-N²-({(1R)-5-[(methylcarbamoyl)amino]-2′,4′-dioxo-2,3-dihydro-3′H-spiro[indene-1,5′-[1,3]oxazolidin]-3′-yl}acetyl)-L-alaninamide

A solution of (R)-2-(5-(3-methylureido)-2′,4′-dioxo-2,3-dihydrospiro[indene-1,5′-oxazolidin]-3′-yl)acetic acid (0.03 g, 0.090 mmol) in N,N-dimethylformamide (0.7 mL) was treated with (S)-2-((4-fluorobenzyl)amino)propanamide (0.019 g, 0.095 mmol), 2-(3H-[1,2,3]triazolo[4,5-b]pyridin-3-yl)-1,1,3,3-tetramethylisouronium hexafluorophosphate(V) (0.038 g, 0.099 mmol) and lastly, N-ethyl-N-isopropylpropan-2-amine (0.036 ml, 0.207 mmol) and the reaction mixture was allowed to stir at room temperature for 18 h. The reaction mixture was directly purified by reverse-phase HPLC to give the title compound. ¹H NMR (400 MHz, DMSO-d₆) δ ppm 8.67 (s, 1H), 7.53 (dd, J=11.6, 1.8 Hz, 1H), 7.47-7.14 (m, 5H), 7.15-7.02 (m, 1H), 6.14-5.97 (m, 1H), 4.97-4.49 (m, 4H), 4.34-4.09 (m, 1H), 3.22-2.88 (m, 2H), 2.71-2.50 (m, 4H), 2.48-2.36 (m, 1H), 1.30-1.06 (m, 3H). MS (ESI⁺⁾ m/z 512 (M+H)⁺.

TABLE 19
The following EXAMPLEs were prepared essentially as described in EXAMPLE 503,
substituting an appropriate amine and appropriate aldehyde in EXAMPLE 503A.

| Ex | Name | NMR | MS |
|---|---|---|---|
| 517 | 3-[(4-fluorobenzyl)({(1R)-5- | ¹H NMR (400 MHz, DMSO-d₆) δ ppm | (ESI(+)) |
| | [(methylcarbamoyl)amino]- | 8.73-8.63 (m, 1H), 7.54 (s, 1H), 7.44- | m/e 526 |
| | 2′,4′-dioxo-2,3-dihydro-3′H- | 7.13 (m, 6H), 7.15-7.04 (m, 1H), 6.94- | (M + H)⁺ |
| | spiro[indene-1,5′- | 6.75 (m, 1H), 6.06 (brs, 1H), 4.97-4.30 | |
| | [1,3]oxazolidin]-3′- | (m, 5H), 3.19-3.05 (m, 1H), 3.05-2.93 | |
| | yl}acetyl)amino]butanamide | (m, 1H), 2.68-2.55 (m, 4H), 2.48-2.21 | |
| | | (m, 3H), 1.13-1.04 (m, 3H) | |
| 599 | N-cyclohexyl-N-(4- | ¹H NMR (400 MHz, DMSO-d₆) δ 8.71 (d, | (ESI(+)) |
| | fluorobenzyl)-2-{(1R)-5- | J = 3.9 Hz, 1H), 7.60-7.52 (m, 1H), 7.37 | m/e 523 |
| | [(methylcarbamoyl)amino]- | (dd, J = 8.5, 5.4 Hz, 1H), 7.33-7.17 (m, | (M + H)⁺ |
| | 2′,4′-dioxo-2,3-dihydro-3′H- | 4H), 7.15-7.06 (m, 1H), 6.10 (s, 1H), | |
| | spiro[indene-1,5′- | 4.75-4.45 (m, 3H), 4.39-4.19 (m, 1H), | |
| | [1,3]oxazolidin]-3′- | 3.96-3.69 (m, 1H), 3.12 (dq, J = 15.2, | |
| | yl}acetamide | 7.2 Hz, 1H), 3.01 (ddt, J = 11.9, 8.2, 3.5 | |
| | | Hz, 1H), 2.64 (t, J = 3.3 Hz, 4H), 2.56- | |
| | | 2.40 (m, 1H), 1.78-1.50 (m, 6H), 1.50- | |
| | | 0.90 (m, 6H) | |
| 600 | N-cyclopentyl-N-(4- | ¹H NMR (400 MHz, DMSO-d₆) δ 8.71 (d, | (ESI(+)) |
| | fluorobenzyl)-2-{(1R)-5- | J = 4.3 Hz, 1H), 7.55 (d, J = 4.9 Hz, 1H), | m/e 509 |
| | [(methylcarbamoyl)amino]- | 7.38-7.17 (m, 5H), 7.13 (t, J = 8.7 Hz, | (M + H)⁺ |
| | 2′,4′-dioxo-2,3-dihydro-3′H- | 1H), 6.39-5.76 (m, 1H), 4.79-4.43 (m, | |
| | spiro[indene-1,5′- | 4H), 4.33 (d, J = 25.4 Hz, 1H), 3.20- | |
| | [1,3]oxazolidin]-3′- | 3.06 (m, 1H), 3.00 (dd, J = 8.0, 3.6 Hz, | |
| | yl}acetamide | 1H), 2.72-2.57 (m, 4H), 2.57-2.40 (m, | |
| | | 1H), 1.92-1.38 (m, 8H) | |
| 607 | N-cyclopropyl-N-(4- | ¹H NMR (400 MHz, DMSO-d₆) δ 8.73 (s, | (ESI(+)) |
| | fluorobenzyl)-2-{(1R)-5- | 1H), 7.56 (d, J = 1.8 Hz, 1H), 7.37-7.20 | m/e 481 |
| | [(methylcarbamoyl)amino]- | (m, 4H), 7.20-7.11 (m, 2H), 6.11 (s, | (M + H)⁺ |
| | 2′,4′-dioxo-2,3-dihydro-3′H- | 1H), 4.77-4.63 (m, 2H), 4.63-4.50 (m, | |
| | spiro[indene-1,5′- | 2H), 3.16-3.09 (m, 1H), 3.02 (ddd, J = | |
| | [1,3]oxazolidin]-3′- | 12.9, 8.8, 4.5 Hz, 1H), 2.87-2.74 (m, | |
| | yl}acetamide | 1H), 2.70-2.64 (m, 4H), 2.56-2.45 (m, | |
| | | 1H), 0.96-0.80 (m, 4H) | |
| 610 | N-(4-fluorobenzyl)-N-(3- | ¹H NMR (400 MHz, DMSO-d₆ 120° C.) δ | (ESI(+)) |
| | fluorophenyl)-2-{(1R)-5- | ppm 8.27 (brs, 1H), 7.50-7.41 (m, 2H), | m/e 535 |
| | [(methylcarbamoyl)amino]- | 7.27-7.01 (m, 9H), 5.90-5.79 (m, 1H), | (M + H)⁺ |
| | 2′,4′-dioxo-2,3-dihydro-3′H- | 4.92 (brs, 2H), 4.22-4.11 (m, 2H), 3.17- | |
| | spiro[indene-1,5′- | 3.06 (m, 1H), 3.05-2.95 (m, 1H), 2.69- | |
| | [1,3]oxazolidin]-3′- | 2.64 (m, 4H), 2.46-2.40 (m, 1H) | |
| | yl}acetamide | | |
| 633 | N-(4-fluorobenzyl)-2-{(1R)-5- | ¹H NMR (400 MHz, DMSO-d₆) δ ppm | (APCI⁺) |
| | [(methylcarbamoyl)amino]- | 8.77-8.65 (m, 1H), 7.58-7.50 (m, 1H), | m/z 561 |
| | 2′,4′-dioxo-2,3-dihydro-3′H- | 7.48-7.38 (m, 1H), 7.37-7.06 (m, 5H), | (M + H)⁺ |
| | spiro[indene-1,5′- | 6.07 (brs, 1H), 4.92-4.61 (m, 3H), 4.61- | |
| | [1,3]oxazolidin]-3′-yl}-N-[1- | 4.19 (m, 2H), 3.83-3.38 (m, 2H), 3.18- | |
| | (methylsulfonyl)propan-2- | 3.06 (m, 1H), 3.06-2.94 (m, 4H), 2.72- | |
| | yl]acetamide | 2.57 (m, 4H), 2.55-2.43 (m, 1H), 1.25- | |
| | | 1.16 (m, 3H) | |
| 634 | N-(4-fluorobenzyl)-2-{(1R)-5- | ¹H NMR (400 MHz, DMSO-d₆, 120° C.) | (ESI(+)) |
| | [(methylcarbamoyl)amino]- | δ ppm 8.27 (brs, J = 3.8 Hz, 1H), 7.54- | m/e 535 |
| | 2′,4′-dioxo-2,3-dihydro-3′H- | 7.46 (m, 2H), 7.32-7.21 (m, 5H), 7.10 (t, | (M + H)⁺ |
| | spiro[indene-1,5′- | J = 8.7 Hz, 2H), 5.86 (brs, 1H), 4.59- | |
| | [1,3]oxazolidin]-3′-yl}-N-[(1- | 4.47 (m, 4H), 4.40 (s, 2H), 3.77 (s, 3H), | |
| | methyl-1H-pyrazol-4- | 3.21-3.06 (m, 1H), 3.06-2.92 (m, 1H), | |
| | yl)methyl]acetamide | 2.70-2.60 (m, 4H), 2.52-2.41 (m, 1H) | |
| 649 | N-(4-fluorobenzyl)-2-{(1R)-5- | ¹H NMR (400 MHz, DMSO-d₆) δ ppm | (ESI(+)) |
| | [(methylcarbamoyl)amino]- | 2.38-2.49 (m, 1H), 2.57-2.72 (m, 4H), | m/e 521 |
| | 2′,4′-dioxo-2,3-dihydro-3′H- | 2.92-3.21 (m, 2H), 3.79 (s, 3H), 4.13- | (M + H)⁺ |
| | spiro[indene-1,5′- | 4.32 (m, 2H), 4.71-4.87 (m, 2H), 6.06- | |
| | [1,3]oxazolidin]-3′-yl}-N-(1- | 6.18 (m, 1H), 7.08-7.21 (m, 2H), 7.19- | |
| | methyl-1H-pyrazol-4- | 7.35 (m, 4H), 7.45 (s, 1H), 7.54 (d, J = 1.9 | |
| | yl)acetamide | Hz, 1H), 7.86 (s, 1H), 8.74 (s, 1H) | |
| 665 | N-(4-fluorobenzyl)-2-{(1R)-5- | ¹H NMR (400 MHz, DMSO-d₆, 120° C.) | (ESI(+)) |
| | [(methylcarbamoyl)amino]- | δ ppm 2.39-2.49 (m, 1H), 2.60-2.74 | m/e 522 |
| | 2′,4′-dioxo-2,3-dihydro-3′H- | (m, 5H), 2.94-3.21 (m, 2H), 4.45-4.80 | (M + H)⁺ |
| | spiro[indene-1,5′- | (m, 5H), 5.76-5.87 (m, 1H), 7.02-7.35 | |
| | [1,3]oxazolidin]-3′-yl}-N-(1,3- | (m, 6H), 7.48 (s, 1H), 7.89 (s, 1H), 8.09- | |
| | oxazol-4-ylmethyl)acetamide | 8.35 (m, 2H) | |
| 676 | N-(4-fluorobenzyl)-N-(trans-3- | ¹H NMR (400 MHz, methanol-d₄) δ 7.51 | (ESI(+)) |
| | hydroxycyclobutyl)-2-{(1R)-5- | (s, 1H), 7.37 (t, J = 7.5 Hz, 1H), 7.30 (t, | m/e 511 |
| | [(methylcarbamoyl)amino]- | J = 4.9 Hz, 1H), 7.23-7.08 (m, 3H), 7.03 | (M + H)⁺ |
| | 2′,4′-dioxo-2,3-dihydro-3′H- | (t, J = 8.6 Hz, 1H), 4.76 (s, 2H), 4.64- | |
| | spiro[indene-1,5′- | 4.53 (m, 1H), 4.47-4.30 (m, 1H), 4.10- | |
| | [1,3]oxazolidin]-3′- | 4.01 (m, 1H), 3.94-3.87 (m, 1H), 3.18 | |
| | yl}acetamide | (dd, J = 15.9, 7.3 Hz, 1H), 3.10-2.98 (m, | |
| | | 1H), 2.76 (s, 3H), 2.65-2.49 (m, 4H), | |
| | | 2.13-2.03 (m, 1H), 2.01-1.89 (m, 1H) | |
| 684 | N-(1-cyanopropan-2-yl)-N-(4- | ¹H NMR (400 MHz, DMSO-d₆, 120° C.) | (ESI(+)) |
| | fluorobenzyl)-2-{(1R)-5- | δ ppm 1H NMR (400 MHz, DMSO-d6, | m/e 508 |
| | [(methylcarbamoyl)amino]- | 120° C.) δ ppm 1.24 (d, J = 6.8 Hz, 3H), | (M + H)⁺ |
| | 2′,4′-dioxo-2,3-dihydro-3′H- | 2.43-2.53 (m, 1H), 2.61-2.72 (m, 4H), | |
| | spiro[indene-1,5′- | 2.74-2.88 (m, 2H), 2.94-3.19 (m, 2H), | |
| | [1,3]oxazolidin]-3′- | 4.36-4.56 (m, 3H), 4.56-4.74 (m, 2H), | |
| | yl}acetamide | 5.65-6.07 (m, 1H), 7.06-7.18 (m, 2H), | |
| | | 7.19-7.31 (m, 2H), 7.31-7.43 (m, 2H), | |
| | | 7.48 (s, 1H), 8.22-8.31 (m, 1H) | |
| 696 | N-(4-fluorobenzyl)-N-(trans-4- | ¹H NMR (400 MHz, methanol-d₄) δ 7.52 | (ESI(+)) |
| | hydroxycyclohexyl)-2-{(1R)-5- | (s, 1H), 7.42-7.33 (m, 2H), 7.24-7.10 | m/e 561 |
| | [(methylcarbamoyl)amino]- | (m, 3H), 7.00 (t, J = 8.7 Hz, 1H), 4.70 (d, | (M + Na)⁺ |
| | 2′,4′-dioxo-2,3-dihydro-3′H- | J = 6.7 Hz, 1H), 4.62 (d, J = 12.0 Hz, | |
| | spiro[indene-1,5′- | 2H), 4.46-4.21 (m, 2H), 3.84 (t, J = 11.6 | |
| | [1,3]oxazolidin]-3′- | Hz, 1H), 3.52-3.37 (m, 1H), 3.22-3.12 | |
| | yl}acetamide | (m, 1H), 3.11-2.98 (m, 1H), 2.81-2.70 | |
| | | (m, 4H), 2.62-2.48 (m, 1H), 1.99-1.86 | |
| | | (m, 2H), 1.81-1.50 (m, 4H), 1.47-1.24 | |
| | | (m, 2H) | |
| 697 | methyl (1R,3S)-3-[(4- | ¹H NMR (400 MHz, methanol-d₄) δ 7.52 | (ESI(+)) |
| | fluorobenzyl)({(1R)-5- | (s, 1H), 7.38 (dd, J = 12.4, 8.4 Hz, 2H), | m/e 603 |
| | [(methylcarbamoyl)amino]- | 7.27-7.07 (m, 3H), 7.00 (t, J = 8.7 Hz, | (M + Na)⁺ |
| | 2′,4′-dioxo-2,3-dihydro-3′H- | 1H), 4.73-4.58 (m, 3H), 4.46-4.29 (m, | |
| | spiro[indene-1,5′- | 2H), 3.95-3.87 (m, 1H), 3.67-3.61 (m, | |
| | [1,3]oxazolidin]-3′- | 3H), 3.24-3.14 (m, 1H), 3.12-2.99 (m, | |
| | yl}acetyl)amino]cyclohexane- | 1H), 2.80-2.70 (m, 4H), 2.60-2.39 (m, | |
| | carboxylate | 2H), 2.05-1.14 (m, 8H) | |
| 712 | N-(4-fluorobenzyl)-N-(4- | ¹H NMR (400 MHz, DMSO-d₆, 120° C.) δ | (APCI⁺) |
| | methoxyphenyl)-2-{(1R)-5- | ppm 8.26 (brs, 1H), 7.48 (brs, 1H), 7.28- | m/z 547 |
| | [(methylcarbamoyl)amino]- | 7.17 (m, 4H), 7.17-7.09 (m, 2H), 7.04 (t, | (M + H)⁺ |
| | 2′,4′-dioxo-2,3-dihydro-3′H- | J = 8.7 Hz, 2H), 6.96 (d, J = 8.4 Hz, 2H), | |
| | spiro[indene-1,5′- | 5.85 (brs, 1H), 4.85 (s, 2H), 4.15-3.98 | |
| | [1,3]oxazolidin]-3′- | (m, 2H), 3.77 (s, 3H), 3.20-2.91 (m, | |
| | yl}acetamide | 2H), 2.73-2.59 (m, 4H), 2.50-2.37 (m, | |
| | | 1H) | |
| 718 | N-(4-fluorobenzyl)-2-{(1R)-5- | ¹H NMR (501 MHz, dimethylsulfoxide- | (ESI(+)) |
| | [(methylcarbamoyl)amino]- | d₆) δ ppm 1.11-1.22 (m, 1H), 1.27-1.37 | m/e 549 |
| | 2′,4′-dioxo-2,3-dihydro-3′H- | (m, 1H), 1.68-1.79 (m, 1H), 1.99-2.12 | (M + H)⁺ |
| | spiro[indene-1,5′- | (m, 1H), 2.43-2.69 (m, 2H), 2.92-3.15 | |
| | [1,3]oxazolidin]-3′-yl}-N-[1- | (m, 2H), 4.43-4.66 (m, 2H), 4.71-4.95 | |
| | (trifluoromethyl)cyclopropyl]acet- | (m, 2H), 5.95-6.17 (m, 1H), 7.05-7.33 | |
| | amide | (m, 6H), 7.49-7.59 (m, 1H), 8.61-8.77 | |
| | | (m, 1H) | |
| 732 | tert-butyl 3-[(4- | ¹H NMR (400 MHz, DMSO-d₆) δ 8.67 (d, | (ESI(+)) |
| | fluorobenzyl)({(1R)-5- | J = 4.5 Hz, 1H), 7.59-7.46 (m, 1H), 7.37- | m/e 596 |
| | [(methylcarbamoyl)amino]- | 7.05 (m, 6H), 6.05 (d, J = 5.2 Hz, 1H), | (M + H)⁺ |
| | 2′,4′-dioxo-2,3-dihydro-3′H- | 5.14-4.60 (m, 3H), 4.38 (d, J = 2.4 Hz, | |
| | spiro[indene-1,5′- | 1H), 4.20-3.73 (m, 4H), 3.20-2.91 (m, | |
| | [1,3]oxazolidin]-3′- | 2H), 2.62 (d, J = 4.2 Hz, 4H), 2.48 (p, J = | |
| | yl}acetyl)amino]azetidine-1- | 1.9 Hz, 1H), 1.34 (s, 9H), 1.26-1.03 (m, | |
| | carboxylate | 1H) | |
| 754 | methyl (1S,3S)-3-[(4- | ¹H NMR (501 MHz, DMSO-d₆) δ 8.69 (s, | (ESI(+)) |
| | fluorobenzyl)({(1R)-5- | 1H), 7.56 (dd, J = 5.5, 4.3 Hz, 1H), 7.40- | m/e 581 |
| | [(methylcarbamoyl)amino]- | 7.17 (m, 5H), 7.15-7.08 (m, 1H), 6.07 | (M + H)⁺ |
| | 2′,4′-dioxo-2,3-dihydro-3′H- | (s, 1H), 4.85 (dd, J = 16.8, 14.4 Hz, 1H), | |
| | spiro[indene-1,5′- | 4.68-4.23 (m, 4H), 4.03-3.95 (m, 1H), | |
| | [1,3]oxazolidin]-3′- | 3.67 (s, 2H), 3.62 (s, 1H), 3.17-3.09 (m, | |
| | yl}acetyl)amino]cyclohexane- | 1H), 2.99 (ddd, J = 16.3, 7.8, 3.6 Hz, 1H), | |
| | carboxylate | 2.93-2.80 (m, 1H), 2.70-2.60 (m, 4H), | |
| | | 2.55-2.45 (m, 1H), 1.98-1.84 (m, 2H), | |
| | | 1.75-1.21 (m, 5H) | |
| 755 | N-(4-fluorobenzyl)-2-{(1R)-5- | ¹H NMR (501 MHz, methanol-d₄) δ 7.51 | (ESI(+)) |
| | [(methylcarbamoyl)amino]- | (s, 1H), 7.43-7.33 (m, 2H), 7.28-7.17 | m/e 547 |
| | 2′,4′-dioxo-2,3-dihydro-3′H- | (m, 2H), 7.13 (t, J = 8.7 Hz, 1H), 7.01 (t, | (M + H)⁺ |
| | spiro[indene-1,5′- | J = 8.7 Hz, 1H), 4.73 (t, J = 12.0 Hz, 1H), | |
| | [1,3]oxazolidin]-3′-yl}-N- | 4.67 (d, J = 13.8 Hz, 2H), 4.54 (dt, J = | |
| | (tetrahydro-2H-pyran-4- | 12.0, 3.9 Hz, 1H), 4.47-4.37 (m, 1H), | |
| | yl)acetamide | 4.14-4.05 (m, 1H), 3.95-3.87 (m, 2H), | |
| | | 3.52-3.38 (m, 2H), 3.24-3.13 (m, 1H), | |
| | | 3.10-2.99 (m, 1H), 2.82-2.70 (m, 4H), | |
| | | 2.59-2.48 (m, 1H), 1.86-1.53 (m, 4H) | |
| 756 | N-(1,1-dioxidotetrahydro-2H- | ¹H NMR (400 MHz, DMSO-d₆) δ 8.69 (s, | (ESI(+)) |
| | thiopyran-4-yl)-N-(4- | 1H), 7.54 (d, J = 14.2 Hz, 1H), 7.40 (dd, | m/e 573 |
| | fluorobenzyl)-2-{(1R)-5- | J = 8.5, 5.5 Hz, 1H), 7.34-7.10 (m, 5H), | (M + H)⁺ |
| | [(methylcarbamoyl)amino]- | 6.08 (s, 1H), 4.85-4.23 (m, 5H), 3.41- | |
| | 2′,4′-dioxo-2,3-dihydro-3′H- | 3.25 (m, 2H), 3.17-2.92 (m, 5H), 2.71- | |
| | spiro[indene-1,5′- | 2.57 (m, 4H), 2.19-2.02 (m, 2H), 1.99- | |
| | [1,3]oxazolidin]-3′- | 1.79 (m, 2H) | |
| | yl}acetamide | | |
| 757 | N-(2,2-difluorocyclopentyl)-N- | ¹H NMR (400 MHz, methanol-d₄) δ 7.51 | (ESI(+)) |
| | (4-fluorobenzyl)-2-{(1R)-5- | (s, 1H), 7.42-7.29 (m, 2H), 7.25-7.08 | m/e 567 |
| | [(methylcarbamoyl)amino]- | (m, 3H), 7.05-6.95 (m, 1H), 5.23-5.14 | (M + H)⁺ |
| | 2′,4′-dioxo-2,3-dihydro-3′H- | (m, 0.5H), 5.05-4.94 (m, 0.5 H), 4.83 | |
| | spiro[indene-1,5′- | (m, 3H), 4.35-4.19 (m, 1H), 3.25-3.12 | |
| | [1,3]oxazolidin]-3′- | (m, 1H), 3.10-2.99 (m, 1H), 2.82-2.69 | |
| | yl}acetamide | (m, 4H), 2.61-2.48 (m, 1H), 2.34-2.05 | |
| | | (m, 2H), 1.99-1.64 (m, 4H) | |

### Example 505

### 2-[5′-(acetylamino)-2,5-dioxo-2′,3′-dihydro-1H-spiro[imidazolidine-4, 1′-inden]-1-yl]-N-[(1S)-1-cyclopropylethyl]-N-(4-fluorobenzyl)acetamide

### Example 505A

### tert-butyl 2-(5′-acetamido-2,5-dioxo-2′,3′-dihydrospiro[imidazolidine-4, 1′-inden]-1-yl)acetate

The title compound was prepared as described in EXAMPLE 281F, substituting N-(2,5-dioxo-2′,3′-dihydrospiro[imidazolidine-4, 1′-inden]-5′-yl)acetamide for (R)-5-bromo-2,3-dihydrospiro[indene-1,5′-oxazolidine]-2′,4′-dione and tert-butyl bromoacetate for (S)-2-bromo-N-(4-fluorobenzyl)-N-(1,1,1-trifluoropropan-2-yl)acetamide.

### Example 505B

### 2-(5′-acetamido-2,5-dioxo-2′,3′-dihydrospiro[imidazolidine-4,1′-inden]-1-yl)acetic acid

The title compound was prepared as described in EXAMPLE 302, substituting tert-butyl 2-(5′-acetamido-2,5-dioxo-2′,3′-dihydrospiro[imidazolidine-4, 1′-inden]-1-yl)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 505C

### 2-[5′-(acetylamino)-2,5-dioxo-2′,3′-dihydro-1H-spiro[imidazolidine-4, 1′-inden]-1-yl]-N-(1-cyclopropylethyl)-N-(4-fluorobenzyl)acetamide

The title compound was prepared as described in EXAMPLE 306, substituting 2-(5′-acetamido-2,5-dioxo-2′,3′-dihydrospiro[imidazolidine-4, 1′-inden]-1-yl)acetic acid for cyclopropanecarboxylic acid and (S)-1-cyclopropyl-N-(4-fluorobenzyl)ethanamine for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆) δ 9.96 (s, 1H), 8.72 (d, J=14.4 Hz, 1H), 7.70-6.96 (m, 7H), 4.72 (s, 1H), 4.59 (d, J=5.3 Hz, 1H), 4.44-4.06 (m, 2H), 3.83-3.59 (m, 1H), 3.00 (q, J=6.7 Hz, 2H), 2.66-2.51 (m, 1H), 2.19 (dq, J=13.7, 8.0 Hz, 1H), 2.04 (s, 3H), 1.39-1.21 (m, 1H), 1.22-1.06 (m, 3H), 1.01-0.09 (m, 6H). MS (ESI⁺) m/z 493 (M+H)⁺.

### Example 510

### N-(4-fluorobenzyl)-2-(5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide

### Example 510A

### 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide

The title compound was prepared as described in EXAMPLE 281F, substituting 5′-bromospiro[imidazolidine-4,1′-indene]-2,3′,5(2′H)-trione for (R)-5-bromo-2,3-dihydrospiro[indene-1,5′-oxazolidine]-2′,4′-dione.

### Example 510B

### N-(4-fluorobenzyl)-2-(5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide

The title compound was prepared as described in EXAMPLE 295, substituting 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.95 (dd, J=9.3, 2.9 Hz, 1H), 8.34 (d, J=5.7 Hz, 1H), 8.16-6.84 (m, 9H), 5.55-4.96 (m, 1H), 5.00-3.99 (m, 6H), 3.18-2.83 (m, 2H), 2.63 (d, 3H), 1.31 (m, 3H). MS (ESI⁺) m/z 615 (M+H)⁺.

### Example 511

### (3R)-3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]pyrrolidine-1-carboxamide

To a suspension of N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide (30 mg, 0.052 mmol) in methylene chloride (2 mL) was added triethylamine (0.036 mL, 0.258 mmol). Trimethylsilyl isocyanate (8.9 mg, 0.077 mmol) was added and the solution was stirred at room temperature overnight. Concentration and reverse phase HPLC gave the title compound. ¹H NMR (400 MHz, methanol-d₄) δ 7.51 (s, 1H), 7.38 (dd, J=16.3, 8.3 Hz, 2H), 7.26-7.10 (m, 3H), 7.02 (t, J=8.5 Hz, 1H), 4.93-4.64 (m, 3H), 4.46 (q, J=16.7 Hz, 2H), 3.71-3.42 (m, 2H), 3.31-3.00 (m, 4H), 2.81-2.69 (m, 4H), 2.58-2.48 (m, 1H), 2.17-2.04 (m, 2H). MS (ESI⁺) m/z 553 (M+H)⁺.

### Example 513

### N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2 S)-1,1,1-trifluoropropan-2-yl]acetamide

N-(4-Fluorobenzyl)-2-(5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide (49 mg, 0.080 mmol) in 2 mL tetrahydrofuran and 2 mL methanol was cooled to 0° C. Sodium tetrahydroborate (18.10 mg, 0.478 mmol) was added and the resulting mixture was stirred at 0° C. for 30 minutes. The reaction mixture was treated with a few drops of acetone to decompose the excess sodium tetrahydroborate, and the mixture was directly purified by silica gel column chromatography to give the title compound. ¹H NMR (501 MHz, DMSO-d₆) δ 8.87-8.56 (m, 1H), 8.25-7.78 (m, 3H), 7.62-7.08 (m, 7H), 5.61-5.48 (m, 1H), 5.46-4.98 (m, 2H), 4.98-4.05 (m, 6H), 2.79-2.58 (m, 3H), 2.52-2.22 (m, 2H), 1.43-1.27 (m, 3H). MS (ESI⁺) m/z 617 (M+H)⁺.

### Example 514

### N-(4-fluorobenzyl)-2-(3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide

N-(4-Fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide (20 mg, 0.032 mmol) in 2 mL dichloromethane was cooled to −78° C., and diethylaminosulfur trifluoride (17.14 μl, 0.130 mmol) was added via syringe. The mixture was stirred at −78° C. for 30 minutes, and was allowed to warm from −78° C. to 0° C. The mixture was concentrated to dryness and purified by silica gel column chromatography to give the title compound. ¹H NMR (501 MHz, DMSO-d₆) δ 9.06 (dd, J=13.6, 2.3 Hz, 1H), 8.24 (d, J=5.5 Hz, 1H), 8.08-7.69 (m, 3H), 7.45-7.05 (m, 6H), 6.15 (ddt, J=57.5, 7.6, 4.0 Hz, 1H), 5.52-4.97 (m, 1H), 4.93-4.01 (m, 6H), 3.22-2.79 (m, 1H), 2.64 (s, 3H), 2.40-2.2 (m, 1H), 1.44-1.23 (m, 3H). MS (ESI⁺) m/z 619 (M+H)⁺.

### Example 515

### 2-(6′-bromo-2,5-dioxo-3′,4′-dihydro-1H, 1′H-spiro[imidazolidine-4,2′-naphthalen]-1-yl)-N-[(1S)-1-cyclopropylethyl]-N-(4-fluorobenzyl)acetamide

The title compound was prepared as described in EXAMPLE 289A-B, substituting 6-bromo-3,4-dihydronaphthalen-2(1H)-one for 5-bromo-1H-inden-2(3H)-one in EXAMPLE 289A and 6′-bromo-3′,4′-dihydro-1′H-spiro[imidazolidine-4,2′-naphthalene]-2,5-dione for 5′-bromo-1′,3′-dihydrospiro[imidazolidine-4,2′-indene]-2,5-dione in EXAMPLE 289B. ¹H NMR (400 MHz, DMSO-d₆, 120° C.) δ ppm 8.11 (brs, 1H), 7.41-7.21 (m, 4H), 7.13-6.95 (m, 3H), 4.62 (s, 2H), 4.29-4.11 (m, 2H), 3.60-3.46 (m, 1H), 3.10 (d, J=16.9 Hz, 1H), 3.01-2.89 (m, 2H), 2.81-2.73 (m, 1H), 2.11-1.96 (m, 1H), 1.96-1.80 (m, 1H), 1.15 (d, J=6.7 Hz, 3H), 1.03-0.82 (m, 1H), 0.56-0.41 (m, 1H), 0.32-0.13 (m, 3H). MS (ESI⁺) m/z 528 (M+H)⁺.

### Example 516

### methyl N′-cyano-N-[(1R)-3′-(2-{(4-fluorobenzyl) [(2S)-1,1,1-trifluoropropan-2-yl]amino}-2-oxoethyl)-2′,4′-dioxo-2,3-dihydrospiro[indene-1,5′-[1,3]oxazolidin]-5-yl]carbamimidothioate

A mixture of 2-[(1R)-5-amino-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide (0.15 g, 0.313 mmol) and dimethyl cyanocarbonimidodithioate (0.05 g, 0.344 mmol) in acetonitrile (1 ml) was heated at 85° C. for 20 hours (no apparent reaction). Pyridine (0.2 mL) was added and the mixture was heated at 110° C. in a sealed 2 mL vial for 18 hours. The reaction mixture was cooled to room temperature and partitioned between ethyl acetate and water. The organic layer was washed with brine, dried over anhydrous sodium sulfate, filtered, and concentrated. The concentrate was purified by silica gel column chromatography to afford the title compound. ¹H NMR (400 MHz, DMSO-d₆, 90° C.) δ ppm 10.07 (brs, 1H), 7.51 (d, J=1.8 Hz, 1H), 7.46-7.28 (m, 4H), 7.20-7.09 (m, 2H), 5.24 (brs, 1H), 4.86 (d, J=17.6 Hz, 1H), 4.79-4.19 (m, 3H), 3.27-3.05 (m, 2H), 2.78-2.62 (m, 4H), 2.62-2.50 (m, 1H), 1.36 (d, J=7.0 Hz, 3H). MS (ESI⁺) m/z 578 (M+H)⁺.

### Example 518

### 2-(6′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide

The title compound was prepared as described in EXAMPLE 296A-B, substituting 2-(6′-bromo-2,5-dioxo-3′,4′-dihydro-1H, 1′H-spiro[imidazolidine-4,2′-naphthalen]-1-yl)-N-[(1S)-1-cyclopropylethyl]-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide. ¹H NMR (400 MHz, DMSO-d₆, 120° C.) δ ppm 8.00 (brs, 1H), 7.41-7.29 (m, 2H), 7.07 (t, J=8.7 Hz, 2H), 6.72 (d, J=8.0 Hz, 1H), 6.46-6.35 (m, 2H), 4.62 (s, 2H), 4.41 (brs, 2H), 4.27-4.11 (m, 2H), 3.67-3.47 (m, 1H), 3.01 (d, J=16.3 Hz, 1H), 2.83-2.68 (m, 2H), 2.63 (d, J=16.2 Hz, 1H), 2.06-1.88 (m, 1H), 1.87-1.73 (m, 1H), 1.15 (d, J=6.7 Hz, 3H), 1.01-0.86 (m, 1H), 0.56-0.41 (m, 1H), 0.32-0.10 (m, 3H). MS (ESI⁺) m/z 465 (M+H)⁺.

### Example 519

### N-(4-fluorobenzyl)-2-{(1R)-5-[(methylcarbamothioyl)amino]-2′,4′-dioxo-2,3-dihydro-3′H-spiro[indene-1,5′-[1,3]oxazolidin]-3′-yl}-N-[(2S)-1,1,1-trifluoropropan-2-yl]acetamide

To a solution of 2-[(1R)-5-amino-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide (60 mg, 0.125 mmol) in 2 ml tetrahydrofuran was added isothiocyanatomethane (13.73 mg, 0.188 mmol) followed by triethylamine (21.8 μl, 0.16 mmol). The reaction mixture was heated at 60° C. overnight and the mixture was purified directly by reverse-phase HPLC to give the title compound. ¹H NMR (400 MHz, DMSO-d₆) δ 9.68 (s, 1H), 7.83 (s, 1H), 7.60-6.99 (m, 7H), 5.56-4.09 (m, 5H), 3.27-2.38 (m, 7H), 1.35 (dd, J=18.9, 6.9 Hz, 3H). MS (ESI) m/e 553 (M+H)⁺.

### Example 522

### 2-(7′-amino-1′-methyl-2,5-dioxo-2′,3′-dihydro-1H,1′H-spiro[imidazolidine-4,4′-quinolin]-1-yl)-N-[(1S)-1-cyclopropylethyl]-N-(4-fluorobenzyl)acetamide

The title compound was prepared as described in EXAMPLE 296A-B, substituting 2-(7′-bromo-1′-methyl-2,5-dioxo-2′,3′-dihydro-1H,1′H-spiro[imidazolidine-4,4′-quinolin]-1-yl)-N-[(1S)-1-cyclopropylethyl]-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide. ¹H NMR (400 MHz, DMSO-d₆) δ 8.78 (d, J=18.4 Hz, 1H), 7.44 (dd, J=8.5, 5.4 Hz, 1H), 7.30 7.17 (m, 2H), 7.09 (dd, J=9.9, 7.2 Hz, 2H), 6.32 (d, J=12.7 Hz, 2H), 4.66 (d, J=49.7 Hz, 2H), 4.46-4.25 (m, 2H), 4.24-4.08 (m, 2H), 3.24 (d, J=9.2 Hz, 1H), 2.87 (d, J=2.6 Hz, 3H), 2.15 (dq, J=9.5, 5.7, 5.0 Hz, 1H), 2.00 (s, 1H), 1.15 (dd, J=36.6, 6.6 Hz, 3H), 0.93 (s, 1H), 0.36 (s, 2H), 0.18 (d, J=33.8 Hz, 2H). MS ESI⁺ 480 (M+H)⁺.

### Example 526

### N-[(1S)-1-cyclopropylethyl]-N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}acetamide

The title compound was prepared as described in EXAMPLE 282, substituting 2-(7′-amino-1′-methyl-2,5-dioxo-2′,3′-dihydro-1H,1′H-spiro[imidazolidine-4,4′-quinolin]-1-yl)-N-[(1S)-1-cyclopropylethyl]-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆) δ 8.71 (d, J=17.2 Hz, 1H), 8.35 (s, 1H), 7.42 (dd, J=8.4, 5.4 Hz, 1H), 7.26 (t, J=7.0 Hz, 1H), 7.19 (t, J=8.7 Hz, 1H), 7.08 (t, J=8.8 Hz, 1H), 7.02-6.92 (m, 1H), 6.88 (d, J=6.3 Hz, 1H), 6.56 (t, J=7.7 Hz, 1H), 5.91 (d, J=4.9 Hz, 1H), 4.70 (s, 1H), 4.58 (s, 1H), 4.45-4.23 (m, 1H), 4.18-4.06 (m, 1H), 3.70 (s, 1H), 3.45-3.32 (m, 2H), 3.18 (d, J=8.1 Hz, 1H), 2.82 (d, J=2.8 Hz, 2H), 2.60 (dd, J=4.7, 1.5 Hz, 2H), 1.13 (dd, J=37.1, 6.6 Hz, 3H), 0.91 (s, 1H), 0.47 (s, 1H), 0.28-0.03 (m, 2H). MS ESI⁺ 537 (M+H)⁺.

### Example 527

### N-[(1S)-1-cyclopropylethyl]-N-(4-fluorobenzyl)-2-{6′-[(methylcarbamoyl)amino]-2,5-dioxo-3′,4′-dihydro-1H,1′H-spiro[imidazolidine-4,2′-naphthalen]-1-yl}acetamide

The title compound was prepared as described in EXAMPLE 282, substituting 2-(6′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆, 120° C.) δ ppm 8.11-8.02 (m, 1H), 7.91 (brs, 1H), 7.41-7.29 (m, 2H), 7.20-7.01 (m, 4H), 6.90 (d, J=8.3 Hz, 1H), 5.79-5.62 (m, 1H), 4.62 (s, 2H), 4.28-4.10 (m, 2H), 3.59-3.47 (m, 1H), 3.08 (d, J=16.5 Hz, 1H), 2.92-2.81 (m, 2H), 2.72 (d, J=16.6 Hz, 1H), 2.65 (d, J=4.6 Hz, 3H), 2.09-1.91 (m, 1H), 1.91-1.77 (m, 1H), 1.15 (d, J=6.7 Hz, 3H), 1.01-0.88 (m, 1H), 0.56-0.41 (m, 1H), 0.31-0.16 (m, 3H). MS (ESI⁺) m/z 522 (M+H)⁺.

### Example 528

### 2-[6′-(acetylamino)-2,5-dioxo-3′,4′-dihydro-1H, 1′H-spiro[imidazolidine-4,2′-naphthalen]-1-yl]-N-[(1S)-1-cyclopropylethyl]-N-(4-fluorobenzyl)acetamide

The title compound was prepared as described in EXAMPLE 346, substituting 2-(6′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (400 MHz, DMSO-d₆, 120° C.) δ ppm 9.31 (brs, 1H), 8.06 (brs, 1H), 7.41-7.30 (m, 3H), 7.26 (dd, J=8.3, 2.2 Hz, 1H), 7.07 (t, J=8.7 Hz, 2H), 6.97 (d, J=8.2 Hz, 1H), 4.62 (s, 2H), 4.28-4.11 (m, 2H), 3.60-3.48 (m, 1H), 3.10 (d, J=16.6 Hz, 1H), 2.97-2.85 (m, 2H), 2.75 (d, J=16.7 Hz, 1H), 2.07-1.97 (m, 4H), 1.92-1.81 (m, 1H), 1.15 (d, J=6.6 Hz, 3H), 1.01-0.88 (m, 1H), 0.56-0.41 (m, 1H), 0.33-0.11 (m, 3H). MS (ESI⁺) m/z 507 (M+H)⁺.

### Example 529

### 2-[(1R)-5-(N″-cyano-N′-methylcarbamimidamido)-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

A solution of methyl N′-cyano-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]oxazolidin]-5-yl]carbamimidothioate (0.055 g, 0.095 mmol) in ethanol (0.3 mL) was treated with methanamine (0.065 ml, 0.476 mmol) (30% in ethanol), and the reaction mixture was stirred at room temperature in a sealed 4 mL vial for 24 hours. The reaction mixture was partitioned between ethyl acetate and water. The organic layer was washed with brine, dried over anhydrous sodium sulfate, filtered, and concentrated. The concentrate was purified by silica gel column chromatography to provide the title compound. ¹H NMR (400 MHz, DMSO-d₆) δ ppm 9.03 (brs, 1H), 7.46-7.07 (m, 8H), 5.50-4.14 (m, 5H), 3.23-2.95 (m, 2H), 2.84-2.74 (m, 3H), 2.74-2.51 (m, 2H), 1.44-1.29 (m, 3H). MS (ESI⁺) m/z 561 (M+H)⁺.

### Example 561

### N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]oxazolidin]-5-yl]tetrahydropyrimidine-1(2H)-carboxamide

### Example 561A

### tert-butyl 3-(((R)-3′-(2-((4-fluorobenzyl)((S)-1,1,1-trifluoropropan-2-yl)amino)-2-oxoethyl)-2′,4′-dioxo-2,3-dihydrospiro[indene-1,5′-oxazolidin]-5-yl)carbamoyl)tetrahydropyrimidine-1(2H)-carboxylate

The title compound was prepared as described in EXAMPLE 282, substituting 2-((R)-5-amino-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and tert-butyl tetrahydropyrimidine-1(2H)-carboxylate for 3,3-difluoroazetidine hydrochloride.

### Example 561B

### N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]oxazolidin]-5-yl]tetrahydropyrimidine-1(2H)-carboxamide

The title compound was prepared as described in EXAMPLE 302, substituting tert-butyl 3-(((R)-3′-(2-((4-fluorobenzyl)((S)-1,1,1-trifluoropropan-2-yl)amino)-2-oxoethyl)-2′,4′-dioxo-2,3-dihydrospiro[indene-1,5′-oxazolidin]-5-yl)carbamoyl)tetrahydropyrimidine-1(2H)-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 9.05 (d, J=2.3 Hz, 1H), 8.66 (s, 2H), 7.52 (d, J=1.9 Hz, 1H), 7.39-7.25 (m, 3H), 7.24-7.17 (m, 2H), 7.17-7.03 (m, 2H), 5.46-4.99 (m, 2H), 4.98-4.66 (m, 4H), 4.52 (dd, J=17.1, 13.9 Hz, 2H), 3.64 (q, J=4.3, 2.8 Hz, 2H), 3.23 (s, 2H), 3.11 (dq, J=15.2, 7.3 Hz, 1H), 2.98 (ddt, J=16.6, 8.6, 4.9 Hz, 1H), 2.61 (ddd, J=15.2, 8.5, 6.7 Hz, 1H), 2.26 (s, 1H), 1.74 (s, 2H), 1.33 (dd, J=16.4, 6.9 Hz, 3H). MS ESI⁺ 592 (M+H)⁺.

### Example 562

### 3-acetyl-N-[(1R)-3′-(2-{(4-fluorobenzyl) [(2S)-1,1,1-trifluoropropan-2-yl]amino}-2-oxoethyl)-2′,4′-dioxo-2,3-dihydrospiro[indene-1,5′-[1,3]oxazolidin]-5-yl]tetrahydropyrimidine-1(2H)-carboxamide

The title compound was prepared as described in EXAMPLE 346, substituting N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]oxazolidin]-5-yl]tetrahydropyrimidine-1(2H)-carboxamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (501 MHz, DMSO-d₆) δ 8.91 (s, 1H), 7.59 (d, J=4.6 Hz, 1H), 7.43-7.08 (m, 5H), 5.46-5.36 (m, 1H), 5.13-4.90 (m, 3H), 4.75 (d, J=18.7 Hz, 1H), 4.56 (dd, J=20.3, 17.0 Hz, 1H), 4.21 (d, J=17.1 Hz, 1H), 3.68 (q, J=4.8, 3.9 Hz, 1H), 3.63-3.57 (m, 2H), 3.20-2.94 (m, 2H), 2.73-2.57 (m, 1H), 2.11 (d, J=3.0 Hz, 2H), 2.05-1.95 (m, 1H), 1.59 (m, 2H), 1.36 (m, 3H), 1.24 (s, 1H), 0.97-0.76 (m, 1H). MS ESI⁺ 634 (M+H)⁺.

### Example 563

### 2-(6-amino-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide

The title compound was prepared as described in EXAMPLE 296A-B, substituting 2-(6-bromo-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide. ¹H NMR (400 MHz, DMSO-d6, 120° C.) δ ppm 8.72 (brs, J=2.4 Hz, 1H), 7.38-7.28 (m, 2H), 7.24 (dd, J=8.6, 5.7 Hz, 1H), 7.17-7.05 (m, 2H), 6.98-6.87 (m, 1H), 6.82 (d, J=2.1 Hz, 1H), 5.26-5.02 (m, 1H), 4.80 (dd, J=17.7, 4.6 Hz, 1H), 4.59 (d, J=17.5 Hz, 1H), 4.47 (d, J=16.7 Hz, 1H), 4.31-4.22 (m, 1H), 3.82 (dd, J=14.3, 2.8 Hz, 1H), 3.64 (dd, J=14.2, 3.9 Hz, 1H), 1.34 (d, J=7.0 Hz, 3H). MS (APCI⁺) m/z 529 (M+H)⁺.

### Example 564

### N-(4-fluorobenzyl)-2-(6-{1-[2-(methylamino)-2-oxoethyl]-1H-pyrazol-4-yl}-1,1-dioxido-2′,5′-dioxo-1′H-spiro[1-benzothiophene-3,4′-imidazolidin]-1′-yl)-N-[(2S)-1,1,1-trifluoropropan-2-yl]acetamide

The title compound was prepared as described in EXAMPLE 295, substituting 2-(6-bromo-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆, 120° C.) δ ppm 8.97-8.89 (m, 1H), 8.28 (s, 1H), 8.04-7.91 (m, 3H), 7.65 (dd, J=8.1, 6.3 Hz, 1H), 7.53 (brs, 1H), 7.38-7.27 (m, 2H), 7.18-7.05 (m, 2H), 5.26-5.06 (m, 1H), 4.89-4.72 (m, 3H), 4.68-4.44 (m, 2H), 4.30 (dd, J=16.7, 6.0 Hz, 1H), 3.96 (dd, J=14.4, 3.9 Hz, 1H), 3.79 (dd, J=14.4, 4.4 Hz, 1H), 2.66 (d, J=4.7 Hz, 3H), 1.40-1.30 (m, 3H). MS (APCI⁺) m/z 651 (M+H)⁺.

### Example 569

### tert-butyl {4-[1-(2-{(4-fluorobenzyl) [(2S)-1,1,1-trifluoropropan-2-yl]amino}-2-oxoethyl)-2,3′,5-trioxo-2′,3′-dihydrospiro[imidazolidine-4,1′-inden]-5′-yl]-1H-pyrazol-1-yl}acetate

The title compound was prepared as described in EXAMPLE 295, substituting 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and tert-butyl 2-(4-(4,4,5,5-tetramethyl-1,3,2-dioxaborolan-2-yl)-1H-pyrazol-1-yl)acetate for 5-(4,4,5,5-tetramethyl-1,3,2-dioxaborolan-2-yl)pyridin-2-ol. ¹H NMR (400 MHz, DMSO-d₆) δ 8.95 (dd, J=9.3, 2.9 Hz, 1H), 8.35 (d, J=6.0 Hz, 1H), 8.17-7.05 (m, 8H), 5.37 (p, J=8.0 Hz, 1H), 5.01-4.01 (m, 6H), 3.16-2.79 (m, 2H), 1.44 (s, 9H), 1.40-1.21 (m, 3H). MS (ESI⁺) m/z 658 (M+H)⁺.

### Example 570

### N-(4-fluorobenzyl)-2-{3′-fluoro-5′-[1-(2-methylpropyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide

### Example 570A

### 2-(5′-bromo-3′-hydroxy-2,5-dioxo-2′,3′-dihydro spiro[imidazolidine-4,1′-inden]-1-yl)-N-(4-fluorobenzyl)-N—((S)-1,1,1-trifluoropropan-2-yl)acetamide

2-(5′-Bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide (400 mg, 0.719 mmol) in 5 mL tetrahydrofuran and 5 mL methanol was cooled to 0° C. Sodium tetrahydroborate (82 mg, 2.157 mmol) was added and the solution was stirred at 0° C. for 30 minutes. The mixture was treated with 0.5 mL acetone to decompose excess sodium tetrahydroborate and then added to water and extracted with ethyl acetate. The organic phase was washed with brine and dried over Na₂SO₄. Filtration and concentration gave the title compound.

### Example 570B

### 2-(5′-bromo-3′-fluoro-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide

The title compound was prepared as described in EXAMPLE 514, substituting 2-(5′-bromo-3′-hydroxy-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide.

### Example 570C

### N-(4-fluorobenzyl)-2-{3′-fluoro-5′-[1-(2-methylpropyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide

The title compound was prepared as described in EXAMPLE 295, substituting 2-(5′-bromo-3′-fluoro-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-isobutyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.58 (s, 1H), 8.07 (s, 1H), 7.81 (s, 1H), 7.74-7.55 (m, 2H), 7.32 (dd, J=8.6, 4.8 Hz, 3H), 7.10 (td, J=8.9, 2.9 Hz, 2H), 6.35-5.93 (m, 1H), 5.15 (p, J=7.9 Hz, 1H), 4.93-4.13 (m, 4H), 3.93 (d, J=6.9 Hz, 2H), 3.04 (dd, J=13.6, 6.5 Hz, 1H), 2.44 (s, 1H), 2.18 (dt, J=13.5, 6.8 Hz, 1H), 1.33 (d, J=6.9 Hz, 3H), 0.90 (d, J=6.7 Hz, 6H). MS (ESI⁺) m/z 604 (M+H)⁺.

### Example 571

### {4-[1-(2-{(4-fluorobenzyl)[(2S)-1,1,1-trifluoropropan-2-yl]amino}-2-oxoethyl)-2,3′,5-trioxo-2′,3′-dihydrospiro[imidazolidine-4,1′-inden]-5′-yl]-1H-pyrazol-1-yl}acetic acid

The title compound was prepared as described in EXAMPLE 302, substituting tert-butyl {4-[1-(2-{(4-fluorobenzyl)[(2S)-1,1,1-trifluoropropan-2-yl]amino}-2-oxoethyl)-2,3′,5-trioxo-2′,3′-dihydrospiro[imidazolidine-4,1′-inden]-5′-yl]-1H-pyrazol-1-yl}acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 13.14 (s, 1H), 8.95 (d, J=8.2 Hz, 1H), 8.35 (d, J=5.7 Hz, 1H), 8.19-6.86 (m, 9H), 5.58-5.01 (m, 1H), 5.05-3.93 (m, 6H), 3.16-2.89 (m, 2H), 1.46-1.24 (m, 3H). MS (ESI⁺) m/z 602 (M+H)⁺.

### Example 579

### 2-(5′-{1-[2-(dimethylamino)-2-oxoethyl]-1H-pyrazol-4-yl}-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

The title compound was prepared as described in EXAMPLE 295, substituting 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and tert-butyl 2-(4-(4,4,5,5-tetramethyl-1,3,2-dioxaborolan-2-yl)-1H-pyrazol-1-yl)acetate for 5-(4,4,5,5-tetramethyl-1,3,2-dioxaborolan-2-yl)pyridin-2-ol. ¹H NMR (400 MHz, DMSO-d₆) δ 8.95 (dd, J=9.3, 2.8 Hz, 1H), 8.42-7.88 (m, 4H), 7.77-7.06 (m, 5H), 5.48-5.00 (m, 3H), 5.00-4.03 (m, 4H), 3.2-2.9 (m, 2H), 3.05-2.87 (s, 3H), 2.85 (s, 3H), 1.51-1.27 (m, 3H). MS (ESI⁺) m/z 629 (M+H)⁺.

### Example 580

### N-(4-fluorobenzyl)-2-(5′-{1-[2-(3-hydroxyazetidin-1-yl)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide

The title compound was prepared as described in EXAMPLE 306, substituting {4-[1-(2-{(4-fluorobenzyl)[(2S)-1,1,1-trifluoropropan-2-yl]amino}-2-oxoethyl)-2,3′,5-trioxo-2′,3′-dihydrospiro[imidazolidine-4,1′-inden]-5′-yl]-1H-pyrazol-1-yl}acetic acid for cyclopropanecarboxylic acid and amine for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. H NMR (400 MHz, DMSO-d₆) δ 8.95 (dd, J=9.5, 2.9 Hz, 1H), 8.32 (d, J=6.1 Hz, 1H), 8.14-7.01 (m, 8H), 5.76 (d, J=6.1 Hz, 1H), 5.52-4.98 (m, 1H), 4.97-3.57 (m, 11H), 3.20-2.89 (m, 2H), 1.54-1.24 (m, 3H). MS (ESI⁺) m/z 657 (M+H)⁺.

### Example 581

### 2-(6-bromo-2′,4,4′-trioxo-3,4-dihydro-2H,3′H-spiro[naphthalene-1,5′-[1,3]oxazolidin]-3′-yl)-N-[(1S)-1-cyclopropyl-2,2,2-trifluoroethyl]-N-(4-fluorobenzyl)acetamide

### Example 581A

### 2-(6-bromo-2′,4′-dioxo-3,4-dihydro-2H-spiro[naphthalene-1,5′-oxazolidin]-3′-yl)-N—((S)-1-cyclopropyl-2,2,2-trifluoroethyl)-N-(4-fluorobenzyl)acetamide

The title compound was prepared as described in EXAMPLE 281F, substituting 6-bromo-3,4-dihydro-2H-spiro[naphthalene-1,5′-oxazolidine]-2′,4′-dione for (R)-5-bromo-2,3-dihydrospiro[indene-1,5′-oxazolidine]-2′,4′-dione and (S)-2-bromo-N-(1-cyclopropyl-2,2,2-trifluoroethyl)-N-(4-fluorobenzyl)acetamide for (S)-2-bromo-N-(4-fluorobenzyl)-N-(1,1,1,1-trifluoropropan-2-yl)acetamide.

### Example 581B

### 2-(6-bromo-2′,4,4′-trioxo-3,4-dihydro-2H,3′H-spiro[naphthalene-1,5′-[1,3]oxazolidin]-3′-yl)-N-[(1S)-1-cyclopropyl-2,2,2-trifluoroethyl]-N-(4-fluorobenzyl)acetamide

An 8 mL vial equipped with a stirbar was charged with 2-(6-bromo-2′,4′-dioxo-3,4-dihydro-2H-spiro[naphthalene-1,5′-oxazolidin]-3′-yl)-N—((S)-1-cyclopropyl-2,2,2-trifluoroethyl)-N-(4-fluorobenzyl)acetamide (200 mg, 0.343 mmol), anhydrous dichloroethane (1.5 mL), dirhodium(II) tetrakis(caprolactam) (4.51 mg, 6.86 μmol) and sodium bicarbonate (14.40 mg, 0.171 mmol). The flask was capped with a septa and connected to an empty balloon to capture the oxygen generated during the course of the reaction. To the mixture was added tert-butyl hydroperoxide (0.286 mL, 1.714 mmol) in one portion via syringe and the reaction mixture was stirred at 40° C. overnight. Concentration and purification by silica gel column chromatography gave the title compound. ¹H NMR (400 MHz, dimethylsulfoxide-d₆) δ ppm 0.14-0.46 (m, 2H), 0.66-0.93 (m, 2H), 1.25-1.35 (m, 1H), 2.59-2.79 (m, 2H), 2.79-2.99 (m, 2H), 4.08-4.67 (m, 3H), 4.80-5.14 (m, 2H), 7.05-7.32 (m, 3H), 7.38-7.56 (m, 2H), 7.94-8.11 (m, 2H). MS (ESI⁺⁾ m/z 599 (M+H)⁺.

### Example 582

### N-(4-fluorobenzyl)-2-{6-[(methylcarbamoyl)amino]-1,1-dioxido-2′,5′-dioxo-1′H-spiro[1-benzothiophene-3,4′-imidazolidin]-1′-yl}-N-[(2S)-1,1,1-trifluoropropan-2-yl]acetamide

The title compound was prepared as described in EXAMPLE 282, substituting 2-(6-amino-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆, 120° C.) δ ppm 8.84 (brs, 1H), 8.72 (brs, 1H), 7.95 (d, J=2.0 Hz, 1H), 7.59 (dt, J=8.6, 2.0 Hz, 1H), 7.47 (dd, J=8.6, 6.4 Hz, 1H), 7.36-7.28 (m, 2H), 7.18-7.06 (m, 2H), 6.04-5.96 (m, 1H), 5.26-5.05 (m, 1H), 4.81 (dd, J=17.6, 5.8 Hz, 1H), 4.66-4.43 (m, 2H), 4.34-4.21 (m, 1H), 3.90 (dd, J=14.3, 3.6 Hz, 1H), 3.73 (dd, J=14.3, 4.2 Hz, 1H), 2.68 (d, J=4.3 Hz, 3H), 1.35 (dd, J=7.0, 1.7 Hz, 3H). MS (ESI⁺) m/z 586 (M+H)⁺.

### Example 584

### N-[(1S)-1-cyclopropyl-2,2,2-trifluoroethyl]-N-(4-fluorobenzyl)-2-{6-[(methylcarbamoyl)amino]-2′,4,4′-trioxo-3,4-dihydro-2H,3′H-spiro[naphthalene-1,5′-[1,3]oxazolidin]-3′-yl}acetamide

### Example 584A

### 2-(6-amino-2′,4,4′-trioxo-3,4-dihydro-2H-spiro[naphthalene-1,5′-oxazolidin]-3′-yl)-N—((S)-1-cyclopropyl-2,2,2-trifluoroethyl)-N-(4-fluorobenzyl)acetamide

The title compound was prepared as described in EXAMPLE 284D-E, substituting 2-(6-bromo-2′,4,4′-trioxo-3,4-dihydro-2H,3′H-spiro[naphthalene-1,5′-[1,3]oxazolidin]-3′-yl)-N-[(1S)-1-cyclopropyl-2,2,2-trifluoroethyl]-N-(4-fluorobenzyl)acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide.

### Example 584B

### N-[(1S)-1-cyclopropyl-2,2,2-trifluoroethyl]-N-(4-fluorobenzyl)-2-{6-[(methylcarbamoyl)amino]-2′,4,4′-trioxo-3,4-dihydro-2H,3′H-spiro[naphthalene-1,5′-[1,3]oxazolidin]-3′-yl}acetamide

The title compound was prepared as described in EXAMPLE 282, substituting 2-(6-amino-2′,4,4′-trioxo-3,4-dihydro-2H-spiro[naphthalene-1,5′-oxazolidin]-3′-yl)-N—((S)-1-cyclopropyl-2,2,2-trifluoroethyl)-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, dimethylsulfoxide-d₆) δ ppm 0.23-0.46 (m, 2H), 0.63-0.88 (m, 2H), 1.20-1.31 (m, 1H), 1.82-1.94 (m, 1H), 2.04-2.25 (m, 3H), 2.62-2.78 (m, 4H), 2.90 (dt, J=17.7, 5.3 Hz, 1H), 4.21-4.43 (m, 2H), 4.49-4.99 (m, 3H), 6.70-6.85 (m, 1H), 7.07-7.19 (m, 2H), 7.28 (dd, J=8.7, 1.5 Hz, 1H), 7.33-7.46 (m, 2H), 7.63-7.73 (m, 1H), 7.96 (dd, J=8.8, 1.5 Hz, 1H). MS (ESI⁺) m/z 657 (M+H)⁺.

### Example 585

### N-(4-fluorobenzyl)-2-{(1S)-6-[(methylcarbamoyl)amino]-2′,4′-dioxo-3,4-dihydro-2H,3′H-spiro[naphthalene-1,5′-[1,3]oxazolidin]-3′-yl}-N-[(2S)-1,1,1-trifluoropropan-2-yl]acetamide

N-(4-Fluorobenzyl)-2-{6-[(methylcarbamoyl)amino]-2′,4′-dioxo-3,4-dihydro-2H,3′H-spiro[naphthalene-1,5′-[1,3]oxazolidin]-3′-yl}-N-[(2S)-1,1,1-trifluoropropan-2-yl]acetamide was purified by chiral SFC to give N-(4-fluorobenzyl)-2-{(1S)-6-[(methylcarbamoyl)amino]-2′,4′-dioxo-3,4-dihydro-2H,3′H-spiro[naphthalene-1,5′-[1,3]oxazolidin]-3′-yl}-N-[(2S)-1,1,1-trifluoropropan-2-yl]acetamide (eluted first) and N-(4-fluorobenzyl)-2-{(1S)-6-[(methylcarbamoyl)amino]-2′,4′-dioxo-3,4-dihydro-2H,3′H-spiro[naphthalene-1,5′-[1,3]oxazolidin]-3′-yl}-N-[(2S)-1,1,1-trifluoropropan-2-yl]acetamide (eluted second). The absolute stereochemistry of the spiro center has been tentatively assigned as shown. Spectral data for the title compound: ¹H NMR (400 MHz, dimethylsulfoxide-d₆) δ ppm 1.33 (dd, J=17.1, 6.9 Hz, 3H), 1.65-2.25 (m, 4H), 2.55-2.64 (m, 3H), 2.65-2.86 (m, 2H), 4.11-4.99 (m, 4H), 5.39 (p, J=7.8 Hz, 1H), 5.96-6.12 (m, 1H), 7.05-7.42 (m, 7H), 8.51-8.66 (m, 1H). MS (ESI⁺) m/z 551 (M+H)⁺.

### Example 586

### N-(4-fluorobenzyl)-2-{(1R)-6-[(methylcarbamoyl)amino]-2′,4′-dioxo-3,4-dihydro-2H,3′H-spiro[naphthalene-1,5′-[1,3]oxazolidin]-3′-yl}-N-[(2S)-1, 1,1-trifluoropropan-2-yl]acetamide

N-(4-Fluorobenzyl)-2-{6-[(methylcarbamoyl)amino]-2′,4′-dioxo-3,4-dihydro-2H,3′H-spiro[naphthalene-1,5′-[1,3]oxazolidin]-3′-yl}-N-[(2S)-1,1,1-trifluoropropan-2-yl]acetamide was purified by chiral SFC to give N-(4-fluorobenzyl)-2-{(1S)-6-[(methylcarbamoyl)amino]-2′,4′-dioxo-3,4-dihydro-2H,3′H-spiro[naphthalene-1,5′-[1,3]oxazolidin]-3′-yl}-N-[(2S)-1,1,1-trifluoropropan-2-yl]acetamide (eluted first) and N-(4-fluorobenzyl)-2-{(1S)-6-[(methylcarbamoyl)amino]-2′,4′-dioxo-3,4-dihydro-2H,3′H-spiro[naphthalene-1,5′-[1,3]oxazolidin]-3′-yl}-N-[(2S)-1,1,1-trifluoropropan-2-yl]acetamide (eluted second). The absolute stereochemistry of the spiro center has been tentatively assigned as shown. Spectral data for the title compound: ¹H NMR (400 MHz, dimethylsulfoxide-d₆) δ ppm 1.33 (dd, J=25.6, 6.9 Hz, 3H), 1.65-2.05 (m, 2H), 2.05-2.29 (m, 2H), 2.54-2.64 (m, 3H), 2.65-2.84 (m, 2H), 4.10-4.98 (m, 4H), 5.38 (p, J=7.8 Hz, 1H), 6.00-6.15 (m, 1H), 7.01-7.45 (m, 7H), 8.46-8.71 (m, 1H). MS (ESI⁺) m/z 551 (M+H)⁺.

### Example 587

### 2-[(1R)-5-(4,5-dihydro-1H-imidazol-2-ylamino)-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

A microwave vessel with stirbar was charged with methyl 2-(methylthio)-4,5-dihydro-1H-imidazole-1-carboxylate (54.5 mg, 0.313 mmol), 2-[(1R)-5-amino-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide (100 mg, 0.209 mmol) and acetic acid (417 μl). The vial was sealed and heated in a microwave (Biotage Initiator®) at 120° C. for 20 minutes. Methanol (417 μl) was added to the reaction and the mixture heated for an additional 15 minutes. The mixture was poured into water and ethyl acetate was added; the separated organic layer was washed with brine, dried over anhydrous magnesium sulfate, filtered and concentrated. Silica gel column chromatography gave the title compound. ¹H NMR (400 MHz, dimethylsulfoxide-d₆) δ ppm 1.33 (dd, J=18.1, 6.9 Hz, 3H), 2.49-2.76 (m, 2H), 2.97-3.23 (m, 2H), 3.21-3.43 (m, 4H), 4.13-5.03 (m, 4H), 5.02-5.45 (m, 1H), 7.05-7.52 (m, 7H). MS (ESI⁺) m/z 548 (M+H)⁺.

### Example 588

### 2-(6-amino-2′,4′-dioxo-3,4-dihydro-2H,3′H-spiro[naphthalene-1,5′-[1,3]oxazolidin]-3′-yl)-N-[(1S)-1-cyclopropyl-2,2,2-trifluoroethyl]-N-(4-fluorobenzyl)acetamide

The title compound was prepared as described in EXAMPLE 284D-E, substituting 2-(6-bromo-2′,4′-dioxo-3,4-dihydro-2H-spiro[naphthalene-1,5′-oxazolidin]-3′-yl)-N—((S)-1-cyclopropyl-2,2,2-trifluoroethyl)-N-(4-fluorobenzyl)acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide. ¹H NMR (400 MHz, dimethylsulfoxide-d₆) δ ppm 0.10-0.44 (m, 2H), 0.59-0.89 (m, 2H), 1.19-1.30 (m, 1H), 1.66-1.83 (m, 1H), 1.83-1.95 (m, 1H), 2.00-2.27 (m, 2H), 2.55-2.75 (m, J=5.2 Hz, 2H), 4.05-4.65 (m, 3H), 4.71-5.08 (m, 2H), 5.32 (s, 2H), 6.27-6.35 (m, 1H), 6.35-6.44 (m, 1H), 6.92-7.50 (m, 5H). MS (ESI⁺) m/z 520 (M+H)⁺.

### Example 593

### 2-[(1R)-5-{[(3-amino-2,2-dimethylpropyl)carbamoyl]amino}-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 302, substituting tert-butyl [3-({[(1R)-3′-(2-{(4-fluorobenzyl) [(2S)-1,1,1-trifluoropropan-2-yl]amino}-2-oxoethyl)-2′,4′-dioxo-2,3-dihydrospiro[indene-1,5′-[1,3]oxazolidin]-5-yl]carbamoyl}amino)-2,2-dimethylpropyl]carbamate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 8.94 (d, J=8.6 Hz, 1H), 7.83-7.08 (m, 9H), 6.76 (q, J=6.1 Hz, 1H), 5.48-4.07 (m, 5H), 3.21-2.89 (m, 4H), 2.75-2.40 (m, 4H), 1.36 (dd, J=17.0, 6.9 Hz, 3H), 0.92 (d, J=2.3 Hz, 6H). MS (ESI) m/e 608 (M+H)⁺.

### Example 595

### methyl 2-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]oxazolidin]-5-yl]amino}-4,5-dihydro-H-imidazole-1-carboxylate

The title compound was isolated as a by-product of EXAMPLE 587. ¹H NMR (400 MHz, dimethylsulfoxide-d₆) δ ppm 1.35 (dd, J=17.6, 6.9 Hz, 3H), 2.51-2.82 (m, 2H), 2.97-3.28 (m, 2H), 3.52-3.74 (m, 3H), 3.83-3.93 (m, 1H), 3.99-5.51 (m, 8H), 7.04-7.62 (m, 6H), 8.40-8.64 (m, 1H), 10.43-10.90 (m, 1H). MS (ESI⁺) m/z 606 (M+H)⁺.

### Example 596

### 2-{5-bromo-6-[(methylcarbamoyl)amino]-2′,4′-dioxo-3,4-dihydro-2H,3′H-spiro[naphthalene-1,5′-[1,3]oxazolidin]-3′-yl}-N-[(1S)-1-cyclopropyl-2,2,2-trifluoroethyl]-N-(4-fluorobenzyl)acetamide

### Example 596A

### N—((S)-1-cyclopropyl-2,2,2-trifluoroethyl)-N-(4-fluorobenzyl)-2-(6-(3-methylureido)-2′,4′-dioxo-3,4-dihydro-2H-spiro[naphthalene-1,5′-oxazolidin]-3′-yl)acetamide

The title compound was prepared as described in EXAMPLE 282, substituting 2-(6-amino-2′,4′-dioxo-3,4-dihydro-2H,3′H-spiro[naphthalene-1,5′-[1,3]oxazolidin]-3′-yl)-N-[(1S)-1-cyclopropyl-2,2,2-trifluoroethyl]-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride.

### Example 596B

### 2-{5-bromo-6-[(methylcarbamoyl)amino]-2′,4′-dioxo-3,4-dihydro-2H,3′H-spiro[naphthalene-1,5′-[1,3]oxazolidin]-3′-yl}-N-[(1S)-1-cyclopropyl-2,2,2-trifluoroethyl]-N-(4-fluorobenzyl)acetamide

To a solution of N—((S)-1-cyclopropyl-2,2,2-trifluoroethyl)-N-(4-fluorobenzyl)-2-(6-(3-methylureido)-2′,4′-dioxo-3,4-dihydro-2H-spiro[naphthalene-1,5′-oxazolidin]-3′-yl)acetamide (180 mg, 0.312 mmol) and potassium peroxymonosulfate (576 mg, 0.937 mmol) in nitromethane (1.5 ml) was added KBr (186 mg, 1.561 mmol) at ambient temperature. The mixture was then stirred at 50° C. overnight. Saturated aqueous Na₂SO₃ (30 mL) was added and the reaction mixture was extracted with ethyl acetate. The combined extracts was washed with saturated aqueous brine and dried over anhydrous magnesium sulfate, filtered and concentrated. Silica gel column chromatography afforded the title compound. ¹H NMR (400 MHz, dimethylsulfoxide-d₆) δ ppm 0.23-0.46 (m, 2H), 0.63-0.88 (m, 2H), 1.20-1.31 (m, 1H), 1.82-1.94 (m, 1H), 2.04-2.25 (m, 3H), 2.62-2.78 (m, 4H), 2.90 (dt, J=17.7, 5.3 Hz, 1H), 4.21-4.43 (m, 2H), 4.49-4.99 (m, 3H), 6.70-6.85 (m, 1H), 7.07-7.19 (m, 2H), 7.28 (dd, J=8.7, 1.5 Hz, 1H), 7.33-7.46 (m, 2H), 7.63-7.73 (m, 1H), 7.96 (dd, J=8.8, 1.5 Hz, 1H). MS (ESI⁺) m/z 657 (M+H)⁺.

### Example 597

### 2-(5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

### Example 597A

### 2-(5′-(1-(2-(dimethylamino)-2-oxoethyl)-1H-pyrazol-4-yl)-3′-hydroxy-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide

The title compound was prepared as described in EXAMPLE 570A, substituting 2-(5′-{1-[2-(dimethylamino)-2-oxoethyl]-1H-pyrazol-4-yl}-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide.

### Example 597B

### 2-(5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

The title compound was prepared as described in EXAMPLE 514, substituting 2-(5′-(1-(2-(dimethylamino)-2-oxoethyl)-1H-pyrazol-4-yl)-3′-hydroxy-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide. ¹H NMR (400 MHz, DMSO-d₆) δ 9.05 (d, J=10.2 Hz, 1H), 8.16 (d, J=4.4 Hz, 1H), 7.95 (d, J=4.7 Hz, 1H), 7.81-7.63 (m, 2H), 7.44-7.01 (m, 5H), 6.15 (d, J=57.6 Hz, 1H), 5.43-4.96 (m, 3H), 4.95-3.97 (m, 4H), 3.04 (s, 3H), 2.85 (s, 3H), 2.77 (q, J=7.6 Hz, 1H), 2.35 (dt, J=26.7, 13.5 Hz, 1H), 1.46-1.17 (m, 3H). MS (ESI⁺) m/z 633 (M+H)⁺.

### Example 598

### 2-{5′-[1-(2-amino-2-oxoethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide

### Example 598A

### 2-(5′-(1-(2-amino-2-oxoethyl)-1H-pyrazol-4-yl)-3′-hydroxy-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide

The title compound was prepared as described in EXAMPLE 570A, substituting 2-{5′-[1-(2-amino-2-oxoethyl)-1H-pyrazol-4-yl]-2,3′,5-trioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide.

### Example 598B

### 2-{5′-[1-(2-amino-2-oxoethyl)-1H-pyrazol-4-yl]-3′-fluoro-2,5-dioxo-2′,3′-dihydro-H-spiro[imidazolidine-4,1′-inden]-1-yl}-N-(4-fluorobenzyl)-N-[(2S)-1,1,1-trifluoropropan-2-yl]acetamide

The title compound was prepared as described in EXAMPLE 514, substituting 2-(5′-(1-(2-amino-2-oxoethyl)-1H-pyrazol-4-yl)-3′-hydroxy-2,5-dioxo-2′,3′-dihydrospiro[imidazolidine-4, 1′-inden]-1-yl)-N-(4-fluorobenzyl)-N—((S)-1,1,1-trifluoropropan-2-yl)acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide. ¹H NMR (400 MHz, DMSO-d₆) δ 9.05 (d, J=10.2 Hz, 1H), 8.23 (d, J=4.2 Hz, 1H), 8.03-7.65 (m, 3H), 7.46-7.02 (m, 5H), 6.15 (dt, J=57.5, 5.5 Hz, 1H), 5.50-4.99 (m, 1H), 4.98-3.95 (m, 6H), 2.82-2.59 (m, 1H), 2.34 (ddd, J=26.3, 10.8, 4.6 Hz, 1H), 1.49-1.19 (m, 3H). MS (ESI⁺) m/z 605 (M+H)⁺.

### Example 601

### N-[(1S)-1-cyclopropyl-2,2,2-trifluoroethyl]-N-(4-fluorobenzyl)-2-{4-hydroxy-6-[(methylcarbamoyl)amino]-2′,4′-dioxo-3,4-dihydro-2H,3′H-spiro[naphthalene-1,5′-[1,3]oxazolidin]-3′-yl}acetamide

The title compound was prepared as described in EXAMPLE 570A, substituting N-[(1S)-1-cyclopropyl-2,2,2-trifluoroethyl]-N-(4-fluorobenzyl)-2-{6-[(methylcarbamoyl)amino]-2′,4,4′-trioxo-3,4-dihydro-2H,3′H-spiro[naphthalene-1,5′-[1,3]oxazolidin]-3′-yl}acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide. ¹H NMR (400 MHz, DMSO-d₆) δ 8.87-8.43 (m, 1H), 7.77-6.91 (m, 7H), 6.05 (dq, J=9.2, 4.5 Hz, 1H), 5.57-5.29 (m, 1H), 5.15-4.02 (m, 6H), 2.64 (d, J=4.3 Hz, 3H), 2.40-1.65 (m, 4H), 1.40-0.09 (m, 5H). MS (ESI) m/e 593 (M+H)⁺.

### Example 604

### N-[(1S)-1-cyclopropyl-2,2,2-trifluoroethyl]-N-(4-fluorobenzyl)-2-{4-fluoro-6-[(methylcarbamoyl)amino]-2′,4′-dioxo-3,4-dihydro-2H,3′H-spiro[naphthalene-1,5′-[1,3]oxazolidin]-3′-yl}acetamide

The title compound was prepared as described in EXAMPLE 514, substituting N-[(1S)-1-cyclopropyl-2,2,2-trifluoroethyl]-N-(4-fluorobenzyl)-2-{4-hydroxy-6-[(methylcarbamoyl)amino]-2′,4′-dioxo-3,4-dihydro-2H,3′H-spiro[naphthalene-1,5′-[1,3]oxazolidin]-3′-yl}acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide. ¹H NMR (400 MHz, DMSO-d₆) δ 8.40 (s, 1H), 7.61-7.55 (m, 1H), 7.49-6.95 (m, 6H), 5.88 (s, 2H), 5.02-4.16 (m, 5H), 2.67 (d, J=4.2 Hz, 3H), 2.45-2.05 (m, 4H), 0.99-0.58 (m, 3H), 0.50-0.16 (m, 2H). MS (ESI) m/e 595 (M+H)⁺.

### Example 605

### 2-{5′-[1-(2-amino-2-oxoethyl)-1H-pyrazol-4-yl]-3′-hydroxy-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide

The title compound was prepared in EXAMPLE 598A. ¹H NMR (400 MHz, DMSO-d₆) δ 9.01-8.73 (m, 1H), 8.16 (d, J=5.4 Hz, 1H), 7.90 (d, J=5.6 Hz, 1H), 7.61-7.08 (m, 9H), 5.62-5.46 (m, 1H), 5.46-4.23 (m, 8H), 2.41-2.23 (m, 2H), 1.51-1.26 (m, 3H). MS (ESI⁺) m/z 603 (M+H)⁺.

### Example 606

### N-{[(1R)-3′-(2-{(4-fluorobenzyl) [(2S)-1,1,1-trifluoropropan-2-yl]amino}-2-oxoethyl)-2′,4′-dioxo-2,3-dihydrospiro[indene-1,5′-[1,3]oxazolidin]-5-yl]carbamoyl}-beta-alanine

The title compound was prepared as described in EXAMPLE 302, substituting tert-butyl N-{[(1R)-3′-(2-{(4-fluorobenzyl) [(2S)-1,1,1-trifluoropropan-2-yl]amino}-2-oxoethyl)-2′,4′-dioxo-2,3-dihydrospiro[indene-1,5′-[1,3]oxazolidin]-5-yl]carbamoyl}-beta-alaninate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, dimethylsulfoxide-d₆) δ ppm 1.34 (dd, J=17.7, 6.9 Hz, 3H), 2.32-2.45 (m, 2H), 2.45-2.71 (m, 2H), 2.88-3.19 (m, 2H), 3.22-3.33 (m, 2H), 4.12-4.63 (m, 2H), 4.67-5.51 (m, 3H), 6.30 (t, J=6.0 Hz, 1H), 7.03-7.43 (m, 6H), 7.47-7.61 (m, 1H), 8.66-8.92 (m, 1H), 12.26 (s, 1H). MS (ESI⁺) m/z 595 (M+H)⁺.

### Example 608

### tert-butyl N-{[(1R)-3′-(2-{(4-fluorobenzyl) [(2S)-1,1,1-trifluoropropan-2-yl]amino}-2-oxoethyl)-2′,4′-dioxo-2,3-dihydrospiro[indene-1,5′-[1,3]oxazolidin]-5-yl]carbamoyl}-beta-alaninate

The title compound was prepared as described in EXAMPLE 282, substituting 2-[(1R)-5-amino-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and tert-butyl 3-aminopropanoate for 3,3-difluoroazetidine hydrochloride. ¹H NMR (400 MHz, dimethylsulfoxide-d₆) δ ppm 1.25-1.49 (m, 12H), 2.37 (td, J=6.6, 2.0 Hz, 2H), 2.42-2.56 (m, 1H), 2.55-2.69 (m, 1H), 2.88-3.20 (m, 2H), 3.23-3.29 (m, 2H), 4.13-4.62 (m, 2H), 4.67-5.49 (m, 3H), 6.23 (t, J=5.8 Hz, 1H), 7.05-7.43 (m, 6H), 7.50-7.59 (m, 1H), 8.68-8.86 (m, 1H). MS (ESI⁺) m/z 651 (M+H)⁺.

### Example 609

### N-(3,5-difluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(2S)-1,1,1-trifluoropropan-2-yl]acetamide

To a solution of (R)-2-(5-(3-methylureido)-2′,4′-dioxo-2,3-dihydrospiro[indene-1,5′-oxazolidin]-3′-yl)acetic acid (59.8 mg, 0.179 mmol) in tetrahydrofuran (0.55 ml) at 0° C. was added 1-chloro-N,N-2-trimethylpropenylamine (22.82 μl, 0.172 mmol). The cooling bath was removed and the mixture was stirred at room temperature for 45 minutes. A solution of (S)—N-(3,5-difluorobenzyl)-1,1,1-trifluoropropan-2-amine (33 mg, 0.138 mmol) with pyridine (13.95 μl, 0.172 mmol) in tetrahydrofuran (0.55 ml) was added dropwise. The reaction mixture was stirred at room temperature overnight then concentrated to remove most of the pyridine. The residue was redissolved in 4 ml of 1:1 ethyl acetate/methanol mixture and concentrated onto silica gel. Silica gel column chromatography afforded the title compound. ¹H NMR (400 MHz, dimethylsulfoxide-d₆) δ ppm 1.35 (dd, J=31.2, 6.9 Hz, 3H), 2.40-2.70 (m, 2H), 2.90-3.19 (m, 2H), 4.18-4.64 (m, 2H), 4.72-5.51 (m, 3H), 5.94-6.18 (m, 1H), 6.85-7.35 (m, 5H), 7.47-7.62 (m, 1H), 8.67 (d, J=4.8 Hz, 1H). MS (ESI⁺) m/z 555 (M+H)⁺.

### Example 611

### 2-[(1S)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide

The title compound was prepared in EXAMPLE 287B. ¹H NMR (400 MHz, DMSO-d₆, Temp=90° C.) δ ppm 7.36 (s, 2H), 7.21-6.97 (m, 3H), 6.56-6.39 (m, 2H), 5.12 (s, 2H), 4.67 (s, 2H), 3.03 (dd, J=15.9, 7.7 Hz, 1H), 2.89 (td, J=8.1, 7.5, 3.7 Hz, 1H), 2.69-2.52 (m, 1H), 2.45-2.27 (m, 1H), 1.41-1.09 (m, 5H), 1.07-0.72 (m, 3H), 0.51 (dt, J=8.9, 4.9 Hz, 1H), 0.38-0.10 (m, 3H); MS (ESI(+)) m/e 474 (M+Na)⁺.

### Example 612

### 2-[(1R)-5-amino-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared in EXAMPLE 309A. ¹H NMR (400 MHz, DMSO-d₆) δ ppm 7.39 (dd, J=8.5, 5.4 Hz, 1H), 7.31-7.18 (m, 2H), 7.18-7.01 (m, 2H), 6.59-6.32 (m, 2H), 5.44 (s, 3H), 5.15-4.67 (m, 2H), 4.56 (dd, J=17.0, 8.9 Hz, 1H), 4.22 (d, J=17.0 Hz, 1H), 3.12-2.79 (m, 2H), 2.68-2.30 (m, 2H), 1.36 (dd, J=20.3, 6.9 Hz, 3H). MS (ESI(+)) m/e 502 (M+Na)⁺.

### Example 613

### ethyl ({(3R)-3-[(4-fluorobenzyl){[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]pyrrolidin-1-yl}sulfonyl)carbamate

The title compound was prepared as described in EXAMPLE 295, substituting ethyl ({(3R)-3-[{[(1R)-5-bromo-2′,4′-dioxo-2,3-dihydro-3′H-spiro[indene-1,5′-[1,3]oxazolidin]-3′-yl]acetyl}(4-fluorobenzyl)amino]pyrrolidin-1-yl}sulfonyl)carbamate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.18 (d, J=8.7 Hz, 1H), 7.90 (d, J=7.3 Hz, 1H), 7.60 (s, 1H), 7.53 (t, J=8.2 Hz, 1H), 7.45 (dd, J=14.9, 8.0 Hz, 1H), 7.34 (dd, J=8.5, 5.3 Hz, 1H), 7.20 (ddd, J=33.5, 17.3, 8.6 Hz, 3H), 4.95-4.81 (m, 1H), 4.76 (d, J=18.7 Hz, 2H), 4.60 (m, 1H), 4.40 (q, J=16.8 Hz, 1H), 4.12 (q, J=7.2 Hz, 3H), 3.86 (s, 3H), 3.77-3.41 (m, 2H), 3.30 (dd, J=12.1, 7.6 Hz, 1H), 3.24-3.14 (m, 1H), 3.09 (dd, J=9.2, 4.8 Hz, 1H), 2.68 (dq, J=14.9, 8.1, 7.7 Hz, 1H), 2.57 (ddd, J=21.8, 9.7, 5.5 Hz, 1H), 1.98 (q, J=9.5, 8.7 Hz, 2H), 1.21-1.17 (m, 3H). MS (ESI⁺) m/z 669 (M+H)⁺.

### Example 614

### 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(3R)-piperidin-3-yl]acetamide

### Example 614A

### (R)-benzyl 3-((4-fluorobenzyl)amino)piperidine-1-carboxylate

The title compound was prepared as described in EXAMPLE 283A, substituting (R)-benzyl 3-aminopiperidine-1-carboxylate for (S)-1-cyclopropylethanamine and 4-fluorobenzaldehyde for benzaldehyde.

### Example 614B

### (R)-benzyl 3-(2-((R)-5-bromo-2′,4′-dioxo-2,3-dihydrospiro[indene-1,5′-oxazolidin]-3′-yl)-N-(4-fluorobenzyl)acetamido)piperidine-1-carboxylate

The title compound was prepared as described in EXAMPLE 306, substituting (R)-2-(5-bromo-2′,4′-dioxo-2,3-dihydrospiro[indene-1,5′-oxazolidin]-3′-yl)acetic acid for cyclopropanecarboxylic acid and (R)-benzyl 3-((4-fluorobenzyl)amino)piperidine-1-carboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 614C

### 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(3R)-piperidin-3-yl]acetamide

The title compound was prepared as described in EXAMPLE 302, substituting (R)-benzyl 3-(2-((R)-5-bromo-2′,4′-dioxo-2,3-dihydrospiro[indene-1,5′-oxazolidin]-3′-yl)-N-(4-fluorobenzyl)acetamido)piperidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate and at 50° C. instead of room temperature. ¹H NMR (500 MHz, DMSO-d₆) δ 7.69 (dd, J=4.9, 1.8 Hz, 1H), 7.56 (ddd, J=8.4, 4.1, 1.8 Hz, 1H), 7.44 (dd, J=8.2, 3.5 Hz, 1H), 7.38 (dd, J=8.6, 5.3 Hz, 1H), 7.29-7.22 (m, 2H), 7.15 (t, J=8.8 Hz, 1H), 4.75 (m, 1H), 4.70 -4.47 (m, 3H), 4.37-4.16 (m, 1H), 3.29-3.15 (m, 2H), 3.15-2.95 (m, 3H), 2.70 (dddd, J=26.9, 15.2, 9.0, 5.8 Hz, 2H), 2.62-2.53 (m, 1H), 1.90-1.60 (m, 4H). MS (ESI⁺) m/z 532 (M+H)⁺.

### Example 615

### ethyl ({(3R)-3-[{[(1R)-5-bromo-2′,4′-dioxo-2,3-dihydro-3′H-spiro[indene-1,5′-[1,3]oxazolidin]-3′-yl]acetyl}(4-fluorobenzyl)amino]piperidin-1-yl}sulfonyl)carbamate

The title compound was prepared as described in EXAMPLE 728, substituting 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(3R)-piperidin-3-yl]acetamide for 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(3R)-pyrrolidin-3-yl]acetamide. ¹H NMR (400 MHz, DMSO-d₆) δ 8.70 (s, 1H), 7.56 (dd, J=10.2, 2.0 Hz, 1H), 7.39 (dd, J=8.5, 5.3 Hz, 1H), 7.32 (t, J=9.0 Hz, 1H), 7.29-7.18 (m, 3H), 7.13 (t, J=8.6 Hz, 1H), 6.09 (p, J=4.8 Hz, 1H), 4.80-4.68 (m, 2H), 4.65-4.51 (m, 1H), 4.49-4.30 (m, 2H), 3.58 (t, J=12.7 Hz, 2H), 3.43-3.26 (m, 2H), 3.16-3.06 (m, 1H), 3.01 (ddt, J=11.6, 7.8, 4.0 Hz, 1H), 2.85 (d, J=9.5 Hz, 3H), 2.82-2.67 (m, 2H), 2.64 (dd, J=4.5, 2.7 Hz, 3H), 1.79-1.56 (m, 3H). MS (ESI⁺) m/z 681 (M+H)⁺.

### Example 616

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[1-(methylsulfonyl)piperidin-4-yl]acetamide

The title compound was prepared as described in EXAMPLE 308, substituting N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-(piperidin-4-yl)acetamide for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide hydrochloride. ¹H NMR (400 MHz, DMSO-d₆) δ 8.70 (s, 1H), 7.56 (dd, J=10.2, 2.0 Hz, 1H), 7.39 (dd, J=8.5, 5.3 Hz, 1H), 7.35-7.18 (m, 4H), 7.13 (t, J=8.6 Hz, 1H), 6.09 (p, J=4.8 Hz, 1H), 4.77-4.67 (m, 2H), 4.64-4.52 (m, 1H), 4.38 (dd, J=15.9, 3.5 Hz, 2H), 3.58 (t, J=12.7 Hz, 2H), 3.44-3.27 (m, 2H), 3.16-3.08 (m, 1H), 3.06-2.96 (m, 1H), 2.85 (d, J=9.4 Hz, 3H), 2.73-2.58 (m, 4H), 2.58-2.41 (m, 1H), 1.81-1.56 (m, 4H). MS (ESI⁺) m/z 624 (M+Na)⁺.

### Example 617

### 2-[(4 S)-5′-{1-[2-(dimethylamino)-2-oxoethyl]-1H-pyrazol-4-yl}-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 295, substituting 2-[(4S)-5′-bromo-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N,N-dimethyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.54 (s, 1H), 8.14 (s, 1H), 7.96 (d, J=8.0 Hz, 1H), 7.90 (s, 1H), 7.83 (s, 1H), 7.64 (d, J=8.1 Hz, 1H), 7.38-7.27 (m, 2H), 7.11 (t, J=8.6 Hz, 2H), 5.18 (dq, J=15.7, 7.9, 6.9 Hz, 1H), 5.05 (s, 2H), 4.92-4.23 (m, 4H), 3.19-2.88 (m, 8H), 1.35 (d, J=7.0 Hz, 3H). MS (ESI⁺) m/z 629 (M+H)⁺.

### Example 618

### 2-[(4S)-5′-{1-[2-(dimethylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 295, substituting 2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N,N-dimethyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.35 (s, 1H), 7.96 (s, 1H), 7.76 (s, 1H), 7.52-7.06 (m, 7H), 5.17 (p, J=7.7 Hz, 1H), 5.02 (s, 2H), 4.87-4.08 (m, 4H), 3.06 (t, J=7.2 Hz, 2H), 2.95 (s, 6H), 2.67-2.54 (m, 1H), 2.25 (dt, J=14.1, 7.8 Hz, 1H), 1.34 (d, J=7.0 Hz, 3H). MS (ESI⁺) m/z 615 (M+H)⁺.

### Example 619

### N-(4-fluorobenzyl)-N-[(2S)-1,1,1-trifluoropropan-2-yl]-2-[(4R)-2,3′,5-trioxo-2′,3′-dihydro-1H-spiro[imidazolidine-4,1′-inden]-1-yl]acetamide

The diastereomeric mixture of N-(4-fluorobenzyl)-N-[(2S)-1,1,1-trifluoropropan-2-yl]-2-(2,3′,5-trioxo-2′,3′-dihydro-1H-spiro[imidazolidine-4,1′-inden]-1-yl)acetamide was separated by chiral SFC to afford N-(4-fluorobenzyl)-N-[(2S)-1,1,1-trifluoropropan-2-yl]-2-[(4R)-2,3′,5-trioxo-2′,3′-dihydro-1H-spiro[imidazolidine-4,1′-inden]-1-yl]acetamide (eluted first) and N-(4-fluorobenzyl)-N-[(2S)-1,1,1-trifluoropropan-2-yl]-2-[(4S)-2,3′,5-trioxo-2′,3′-dihydro-1H-spiro[imidazolidine-4,1′-inden]-1-yl]acetamide (eluted second). The absolute configuration was assigned by single crystal x-ray diffraction and by analogy to related analogs. ¹H NMR (400 MHz, DMSO-d₆) δ 8.84 (s, 1H), 7.97-7.53 (m, 4H), 7.47-7.04 (m, 4H), 5.37 (p, J=7.9 Hz, 1H), 4.94-4.02 (m, 4H), 3.20-2.79 (m, 2H), 1.32 (dd, J=25.9, 6.9 Hz, 3H). MS (ESI⁺) m/z 478 (M+H)⁺.

### Example 620

### N-(4-fluorobenzyl)-N-[(2S)-1,1,1-trifluoropropan-2-yl]-2-[(4S)-2,3′,5-trioxo-2′,3′-dihydro-1H-spiro[imidazolidine-4,1′-inden]-1-yl]acetamide

The diastereomeric mixture of N-(4-fluorobenzyl)-N-[(2S)-1,1,1-trifluoropropan-2-yl]-2-(2,3′,5-trioxo-2′,3′-dihydro-1H-spiro[imidazolidine-4,1′-inden]-1-yl)acetamide was separated by chiral SFC to afford N-(4-fluorobenzyl)-N-[(2S)-1,1,1-trifluoropropan-2-yl]-2-[(4R)-2,3′,5-trioxo-2′,3′-dihydro-1H-spiro[imidazolidine-4,1′-inden]-1-yl]acetamide (eluted first) and N-(4-fluorobenzyl)-N-[(2S)-1,1,1-trifluoropropan-2-yl]-2-[(4S)-2,3′,5-trioxo-2′,3′-dihydro-1H-spiro[imidazolidine-4,1′-inden]-1-yl]acetamide (eluted second). The absolute configuration was assigned by single crystal x-ray diffraction and by analogy to related analogs. ¹H NMR (400 MHz, DMSO-d₆) δ 8.95 (s, 1H), 7.97-7.59 (m, 4H), 7.40-7.04 (m, 4H), 5.22 (dp, J=121.9, 7.5 Hz, 1H), 4.97-3.94 (m, 4H), 3.20-2.82 (m, 2H), 1.33 (dd, J=21.7, 6.9 Hz, 3H). MS (ESI⁺) m/z 478 (M+H)⁺.

### Example 621

### N-(4-fluorobenzyl)-2-[5′-(1-methyl-1H-pyrazol-4-yl)-2,3′,5-trioxo-2′,3′-dihydro-1H-spiro[imidazolidine-4,1′-inden]-1-yl]-N-[(2S)-1,1,1-trifluoropropan-2-yl]acetamide

### Example 621A

### 2-(5′-bromo-2,3′,5-trioxo-2′,3′-dihydrospiro[imidazolidine-4,1′-inden]-1-yl)-N-(4-fluorobenzyl)-N-((2S)-1,1,1-trifluoropropan-2-yl)acetamide

The title compound was prepared as described in EXAMPLE 281F, substituting 5′-bromospiro[imidazolidine-4,1′-indene]-2,3′,5(2′H)-trione for (R)-5-bromo-2,3-dihydrospiro[indene-1,5′-oxazolidine]-2′,4′-dione.

### Example 621B

### N-(4-fluorobenzyl)-2-[5′-(1-methyl-1H-pyrazol-4-yl)-2,3′,5-trioxo-2′,3′-dihydro-1H-spiro[imidazolidine-4,1′-inden]-1-yl]-N-[(2 S)-1,1,1-trifluoropropan-2-yl]acetamide

The title compound was prepared as described in EXAMPLE 295, substituting 2-(5′-bromo-2,3′,5-trioxo-2′,3′-dihydrospiro[imidazolidine-4,1′-inden]-1-yl)-N-(4-fluorobenzyl)-N-((2S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.65 (s, 1H), 8.19 (s, 1H), 8.05-7.80 (m, 3H), 7.78-7.57 (m, 1H), 7.32 (d, J=6.6 Hz, 2H), 7.13 (t, J=8.6 Hz, 2H), 5.44-3.98 (m, 5H), 3.87 (s, 3H), 3.08 (dd, J=18.5, 1.7 Hz, 1H), 2.92 (dd, J=18.5, 3.1 Hz, 1H), 1.34 (dd, J=7.1, 2.7 Hz, 3H). MS (ESI⁺) m/z 558 (M+H)⁺.

### Example 622

### N-(4-fluorobenzyl)-2-{6-fluoro-5-[(methylcarbamoyl)amino]-2′,4′-dioxo-2,3-dihydro-3′H-spiro[indene-1,5′-[1,3]oxazolidin]-3′-yl}-N-[(2S)-1,1,1-trifluoropropan-2-yl]acetamide

The title compound was prepared as described in EXAMPLE 282, substituting 2-(5-amino-6-fluoro-2′,4′-dioxo-2,3-dihydro-3′H-spiro[indene-1,5′-[1,3]oxazolidin]-3′-yl)-N-(4-fluorobenzyl)-N-[(2S)-1,1,1-trifluoropropan-2-yl]acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆) δ 8.47 (s, 1H), 8.19 (d, J=7.4 Hz, 1H), 7.38 (dd, J=8.5, 5.5 Hz, 1.4H), 7.26 (dd, J=17.5, 9.5 Hz, 3H), 7.13 (td, J=8.8, 5.1 Hz, 0.6H), 6.57 (d, J=2.9 Hz, 1H), 5.41 (dd, J=15.4, 7.5 Hz, 0.6H), 5.12-4.71 (m, 2.4H), 4.52 (ddd, J=33.6, 22.2, 16.5 Hz, 1.4H), 4.20 (dd, J=17.1, 3.9 Hz, 0.6H), 3.11 (dt, J=14.7, 7.4 Hz, 1H), 3.05-2.93 (m, 1H), 2.74-2.51 (m, 5H), 1.42-1.31 (m, 3H). MS (ESI⁺) m/z 555 (M+H)⁺.

### Example 623

### N-(4-fluorobenzyl)-2-[3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 570A, substituting N-(4-fluorobenzyl)-2-[5′-(1-methyl-1H-pyrazol-4-yl)-2,3′,5-trioxo-2′,3′-dihydro-1H-spiro[imidazolidine-4,1′-inden]-1-yl]-N-[(2S)-1,1,1-trifluoropropan-2-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide. ¹H NMR (400 MHz, DMSO-d₆) δ 8.47 (s, 1H), 8.03 (s, 1H), 7.77 (s, 1H), 7.64-7.40 (m, 2H), 7.43-7.04 (m, 5H), 5.44-4.16 (m, 7H), 3.86 (s, 3H), 2.63-2.22 (m, 2H), 1.35 (d, J=7.0 Hz, 3H). MS (ESI⁺) m/z 560 (M+H)⁺.

### Example 624

### 2-(5-amino-6-fluoro-2′,4′-dioxo-2,3-dihydro-3′H-spiro[indene-1,5′-[1,3]oxazolidin]-3′-yl)-N-(4-fluorobenzyl)-N-[(2S)-1,1,1-trifluoropropan-2-yl]acetamide

The title compound was prepared as described in EXAMPLE 284D-E, substituting 2-(5-bromo-6-fluoro-2′,4′-dioxo-2,3-dihydro-3′H-spiro[indene-1,5′-[1,3]oxazolidin]-3′-yl)-N-(4-fluorobenzyl)-N-[(2S)-1,1,1-trifluoropropan-2-yl]acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide. ¹H NMR (400 MHz, DMSO-d₆) δ 7.44-6.94 (m, 5H), 6.67 (d, J=8.3 Hz, 1H), 5.51 (s, 2H), 5.45-5.31 (m, 0.7H), 5.12-4.37 (m, 3.6H), 4.17 (dd, J=17.1, 2.6 Hz, 0.7H), 2.99 (dt, J=14.7, 7.2 Hz, 1H), 2.92-2.78 (m, 1H), 2.67-2.53 (m, 1H), 2.48-2.37 (m, 1H), 1.41-1.27 (m, 3H). MS (ESI⁺) m/z 453.9 (M-CO2+H)⁺.

### Example 625

### 2-(7′-bromo-1′-methyl-2,5-dioxo-2′,3′-dihydro-1H,1′H-spiro[imidazolidine-4,4′-quinolin]-1-yl)-N-[(1S)-1-cyclopropyl-2,2,2-trifluoroethyl]-N-(4-fluorobenzyl)acetamide

The title compound was prepared as described in EXAMPLE 348, substituting 7-bromo-2,3-dihydroquinolin-4(1H)-one for 2,3-dihydroquinolin-4(1H)-one in EXAMPLE 348A and (S)—N-benzyl-2-bromo-N-(1-cyclopropyl-2,2,2-trifluoroethyl)acetamide for (S)-2-bromo-N-(4-fluorobenzyl)-N-(1,1,1-trifluoropropan-2-yl)acetamide in EXAMPLE 348C. ¹H NMR (500 MHz, DMSO-d₆) δ 8.85 (d, J=23.7 Hz, 1H), 7.44 (dd, J=8.5, 5.5 Hz, 1H), 7.27 (dd, J=8.5, 5.6 Hz, 1H), 7.24-7.15 (m, 1H), 7.15-7.02 (m, 2H), 6.82 (t, J=2.3 Hz, 1H), 6.75 (ddd, J=8.3, 6.2, 1.8 Hz, 1H), 4.72 (s, 1H), 4.62-4.55 (m, 1H), 4.35 (ddd, J=71.1, 16.6, 7.4 Hz, 1H), 4.23-4.06 (m, 1H), 3.49 (ddd, J=11.9, 7.8, 4.1 Hz, 1H), 3.27 (dd, J=10.6, 7.3 Hz, 1H), 2.95-2.84 (m, 4H), 2.73 (s, 1H), 2.20-2.09 (m, 1H), 2.08-1.93 (m, 1H), 1.19 (dd, J=6.5, 3.0 Hz, 1H), 1.10 (d, J=6.8 Hz, 2H), 0.56-0.42 (m, 1H), 0.40-0.06 (m, 3H). MS ESI⁺ 570 (M+H)⁺.

### Example 628

### 2-(5-bromo-6-fluoro-2′,4′-dioxo-2,3-dihydro-3′H-spiro[indene-1,5′-[1,3]oxazolidin]-3′-yl)-N-(4-fluorobenzyl)-N-[(2S)-1,1,1-trifluoropropan-2-yl]acetamide

The title compound was prepared as described in EXAMPLE 281F, substituting 5-bromo-6-fluoro-2,3-dihydrospiro[indene-1,5′-oxazolidine]-2′,4′-dione for (R)-5-bromo-2,3-dihydrospiro[indene-1,5′-oxazolidine]-2′,4′-dione. ¹H NMR (400 MHz, DMSO-d₆) δ 7.79 (d, J=6.5 Hz, 1H), 7.51-7.00 (m, 5H), 5.37 (dt, J=15.3, 7.6 Hz, 0.8H), 5.11-4.01 (m, 5.2H), 3.22-2.94 (m, 2H), 2.68-2.48 (m, 2H), 1.43-1.24 (m, 3H). MS (ESI⁺) m/z 563 (M+H)⁺.

### Example 629

### N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}-N-[(2S)-1,1,1-trifluoropropan-2-yl]acetamide

### Example 629A

### 2-(7′-bromo-1′-methyl-2,5-dioxo-2′,3′-dihydro-1′H-spiro[imidazolidine-4,4′-quinolin]-1-yl)-N-(4-fluorobenzyl)-N—((S)-1,1,1-trifluoropropan-2-yl)acetamide

The title compound was prepared as described in EXAMPLE 281F, substituting 7′-bromo-1′-methyl-2′,3′-dihydro-1′H-spiro[imidazolidine-4,4′-quinoline]-2,5-dione for (R)-5-bromo-2,3-dihydrospiro[indene-1,5′-oxazolidine]-2′,4′-dione.

### Example 629B

### 2-(7′-amino-1′-methyl-2,5-dioxo-2′,3′-dihydro-1′H-spiro[imidazolidine-4,4′-quinolin]-1-yl)-N-(4-fluorobenzyl)-N—((S)-1,1,1-trifluoropropan-2-yl)acetamide

The title compound was prepared as described in EXAMPLE 296A-B, substituting 2-(7′-bromo-1′-methyl-2,5-dioxo-2′,3′-dihydro-1′H-spiro[imidazolidine-4,4′-quinolin]-1-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide.

### Example 629C

### N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}-N-[(2S)-1,1,1-trifluoropropan-2-yl]acetamide

The title compound was prepared as described in EXAMPLE 282, substituting 2-(7′-amino-1′-methyl-2,5-dioxo-2′,3′-dihydro-1′H-spiro[imidazolidine-4,4′-quinolin]-1-yl)-N-(4-fluorobenzyl)-N—((S)-1,1,1-trifluoropropan-2-yl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆) δ 8.76 (d, J=13.1 Hz, 1H), 8.35 (s, 1H), 7.37 (t, J=6.6 Hz, 1H), 7.21 (t, J=8.7 Hz, 2H), 7.09 (q, J=8.2 Hz, 1H), 7.00-6.83 (m, 2H), 6.55 (t, J=9.5 Hz, 1H), 5.91 (d, J=4.9 Hz, 1H), 5.36 (s, 1H), 4.93-4.62 (m, 2H), 4.50-4.22 (m, 2H), 4.12-3.94 (m, 1H), 3.18 (d, J=9.8 Hz, 1H), 2.82 (d, J=2.8 Hz, 3H), 2.60 (d, J=4.5 Hz, 2H), 2.11 (t, J=11.3 Hz, 1H), 1.95 (s, 1H), 1.40-1.20 (m, 3H). MS ESI⁺ 565 (M+H)⁺.

### Example 630

### N-(4-fluorobenzyl)-2-[(3′R,4R)-3′-hydroxy-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 570A, substituting N-(4-fluorobenzyl)-N-[(2S)-1,1,1-trifluoropropan-2-yl]-2-[(4R)-2,3′,5-trioxo-2′,3′-dihydro-1H-spiro[imidazolidine-4,1′-inden]-1-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide. ¹H NMR (400 MHz, DMSO-d₆) δ 8.35 (s, 1H), 7.54-7.22 (m, 6H), 7.11 (t, J=8.6 Hz, 2H), 5.31 (q, J=6.6 Hz, 1H), 5.19 (p, J=7.8 Hz, 1H), 4.93-4.19 (m, 5H), 2.55-2.30 (m, 2H), 1.35 (d, J=7.1 Hz, 3H). MS (ESI⁻) m/z 478 (M−H)⁻.

### Example 635

### N-[(1S)-1-cyclopropylethyl]-N-(4-fluorobenzyl)-2-{4-methoxy-5-[(methylcarbamoyl)amino]-2′,4′-dioxo-2,3-dihydro-3′H-spiro[indene-1,5′-[1,3]oxazolidin]-3′-yl}acetamide

### Example 635A

### 4-hydroxy-2,3-dihydro-1H-inden-1-one

A mixture of aluminium chloride (27 g, 202 mmol), sodium chloride (5.92 g, 101 mmol) and chroman-2-one (5.0 g, 33.7 mmol) was stirred at 210° C. for 2 hours. The mixture was cooled to room temperature and poured slowly into ice water. The precipitate was filtered, triturated with methanol and filtered again to give the title compound.

### Example 635B

### 4-methoxy-2,3-dihydro-1H-inden-1-one

A mixture of 4-hydroxy-2,3-dihydro-1H-inden-1-one (200 mg, 1.35 mmol), K₂CO₃ (560 mg, 4.05 mmol) and iodomethane (958 mg, 6.75 mmol) in N,N-dimethylformamide (2 mL) was stirred at 50° C. for 3 hours. The mixture was diluted with ethyl acetate and washed with water. The organic layer was concentrated and the residue was purified by silica gel column chromatography to afford the title compound.

### Example 635C

### 4-methoxy-5-nitro-2,3-dihydro-1H-inden-1-one

To a solution of 4-methoxy-2,3-dihydro-1H-inden-1-one (81 mg, 0.5 mmol) in acetonitrile (5 ml) cooled to 0° C. in an ice bath was added trifluoroacetic acid (525 mg, 2.5 mmol), followed by KNO₃ (60.6 mg, 0.6 mmol). After approximately 5 minutes, the reaction was allowed to warm to room temperature. The reaction mixture was cooled to 0° C. in an ice bath and quenched with saturated NaHCO₃ (aq), 1N NaOH (aq), and ice until pH=9-10 was achieved. The aqueous mixture was extracted with ethyl acetate and the organic layers were washed with brine, dried over Na₂SO₄, filtered, and concentrated. The residue was purified by silica gel column chromatography to give the title compound.

### Example 635D

### 4-methoxy-5-nitro-1-((trimethylsilyl)oxy)-2,3-dihydro-1H-indene-1-carbonitrile

To a stirring solution of 4-methoxy-5-nitro-2,3-dihydro-1H-inden-1-one (1035 mg, 5 mmol) in dichloromethane (20 mL) was added 4-methylmorpholine-4-oxide (351 mg, 3 mmol) and followed by addition of trimethylsilyl cyanide (1487 mg, 15 mmol) dropwise. The mixture was refluxed overnight. The residue was slurried in 30 mL of petroleum ether for 15 minutes and filtered through a pad of diatomaceous earth. The filtrate was concentrated to obtain the title compound which was used without purification.

### Example 635E

### ethyl 1-hydroxy-4-methoxy-5-nitro-2,3-dihydro-1H-indene-1-carboxylate

A three neck 500 mL flask was fit with hastalloy thermocouple, HCl gas in (diffussor) and HCl out, vented out the back of the hood. 4-Methoxy-5-nitro-1-((trimethylsilyl)oxy)-2,3-dihydro-1H-indene-1-carbonitrile (3.3 g, 10.77 mmol) was taken up in ethanol (10 mL) and cooled to 0° C. HCl (g) (14.53 g, 399 mmol) was bubbled in carefully over 4 hours while the temperature was kept below 25° C. with an ice bath. After stirring for an additional hour, the solvent was concentrated and the residue was purified by silica gel column chromatography to provide the title compound.

### Example 635F

### 4-methoxy-5-nitro-2,3-dihydrospiro[indene-1,5′-oxazolidine]-2′,4′-dione

To the stirring solution of ethyl 1-hydroxy-4-methoxy-5-nitro-2,3-dihydro-1H-indene-1-carboxylate (5 g, 17.78 mmol) in dichloromethane (100 mL) at 0° C. was added trichloroacetyl isocyanate (5.02 g, 26.7 mmol) dropwise. After the addition, the resulting mixture was allowed to stir at room temperature for 30 minutes. The excess trichloroacetyl isocyanate was quenched with methanol (10 mL) and the reaction mixture was concentrated. The residue was dissolved in ethanol (100 mL) and treated with triethylamine (3.6 g, 35.6 mmol). The reaction mixture was then heated to reflux overnight. The solvent was removed and the residue was purified by silica gel column chromatography to afford the title compound.

### Example 635G

### N—((S)-1-cyclopropylethyl)-N-(4-fluorobenzyl)-2-(4-methoxy-5-nitro-2′,4′-dioxo-2,3-dihydrospiro[indene-1,5′-oxazolidin]-3′-yl)acetamide

The title compound was prepared as described in EXAMPLE 281F, substituting 4-methoxy-5-nitro-2,3-dihydrospiro[indene-1,5′-oxazolidine]-2′,4′-dione for (R)-5-bromo-2,3-dihydrospiro[indene-1,5′-oxazolidine]-2′,4′-dione and (S)-2-bromo-N-(1-cyclopropylethyl)-N-(4-fluorobenzyl)acetamide for (S)-2-bromo-N-(4-fluorobenzyl)-N-(1,1,1-trifluoropropan-2-yl)acetamide.

### Example 635H

### 2-(5-amino-4-methoxy-2′,4′-dioxo-2,3-dihydrospiro[indene-1,5′-oxazolidin]-3′-yl)-N—((S)-1-cyclopropylethyl)-N-(4-fluorobenzyl)acetamide

N—((S)-1-Cyclopropylethyl)-N-(4-fluorobenzyl)-2-(4-methoxy-5-nitro-2′,4′-dioxo-2,3-dihydrospiro[indene-1,5′-oxazolidin]-3′-yl)acetamide (0.15 g, 0.293 mmol) and tetrahydrofuran (10 ml) were added to 10% Pd/C, dry (0.034 g, 0.032 mmol) in a 50 ml pressure bottle and stirred for 1 hour at 60 psi and room temperature. The suspension was filtered and concentrated to give the title compound.

### Example 635I

### N-[(1S)-1-cyclopropylethyl]-N-(4-fluorobenzyl)-2-{4-methoxy-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 282, substituting 2-(5-amino-4-methoxy-2′,4′-dioxo-2,3-dihydrospiro[indene-1,5′-oxazolidin]-3′-yl)-N—((S)-1-cyclopropylethyl)-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆, 120° C.) δ ppm 8.04 (d, J=8.4 Hz, 1H), 7.79 (brs, 1H), 7.42-7.31 (m, 2H), 7.15-6.98 (m, 3H), 6.53 (brs, 1H), 4.67 (s, 2H), 4.49-4.29 (m, 2H), 3.82 (s, 3H), 3.68-3.49 (m, 1H), 3.30-3.04 (m, 2H), 2.76-2.60 (m, 4H), 2.54-2.46 (m, 1H), 1.18 (d, J=6.6 Hz, 3H), 1.04-0.89 (m, 1H), 0.58-0.43 (m, 1H), 0.34-0.17 (m, 3H). MS (ESI⁺) m/z 539 (M+H)⁺.

### Example 636

### 2-[5-(acetylamino)-4-methoxy-2′,4′-dioxo-2,3-dihydro-3′H-spiro[indene-1,5′-[1,3]oxazolidin]-3′-yl]-N-[(1S)-1-cyclopropylethyl]-N-(4-fluorobenzyl)acetamide

The title compound was prepared as described in EXAMPLE 346, substituting 2-(5-amino-4-methoxy-2′,4′-dioxo-2,3-dihydrospiro[indene-1,5′-oxazolidin]-3′-yl)-N—((S)-1-cyclopropylethyl)-N-(4-fluorobenzyl)acetamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (400 MHz, DMSO-d₆, 120° C.) δ ppm 8.83 (brs, 1H), 7.86 (d, J=8.3 Hz, 1H), 7.42-7.30 (m, 2H), 7.14-7.02 (m, 3H), 4.67 (s, 2H), 4.49-4.28 (m, 2H), 3.85 (s, 3H), 3.58 (brs, 1H), 3.33-3.07 (m, 2H), 2.74-2.64 (m, 1H), 2.56-2.48 (m, 1H), 2.09 (s, 3H), 1.18 (d, J=6.6 Hz, 3H), 1.04-0.91 (m, 1H), 0.57-0.43 (m, 1H), 0.34-0.16 (m, 3H). MS (ESI⁺) m/z 523.9 (M+H)⁺.

### Example 637

### N-(4-fluorobenzyl)-2-[(3′S,4R)-3′-fluoro-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 514, substituting N-(4-fluorobenzyl)-2-[(3′R,4R)-3′-hydroxy-2,5-dioxo-2′,3′-dihydro-1H-spiro[imidazolidine-4,1′-inden]-1-yl]-N-[(2S)-1,1,1-trifluoropropan-2-yl]acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4, 1′-inden]-1-yl)-N-[(2S)-1,1,1-trifluoropropan-2-yl]acetamide. ¹H NMR (400 MHz, DMSO-d₆) δ 8.55 (s, 1H), 7.61-7.03 (m, 8H), 6.11 (dt, J=57.7, 5.6 Hz, 1H), 5.16 (dt, J=15.4, 8.1 Hz, 1H), 4.85-4.16 (m, 4H), 3.15 (dt, J=14.8, 7.2 Hz, 1H), 2.42-2.17 (m, 1H), 1.33 (d, J=7.0 Hz, 3H). MS (ESI⁺) m/z 482 (M+H)⁺.

### Example 638

### 2-(7′-bromo-2,2′,5-trioxo-2′,3′-dihydro-1H,1′H-spiro[imidazolidine-4,4′-quinolin]-1-yl)-N-(4-fluorobenzyl)-N-[(2S)-1,1,1-trifluoropropan-2-yl]acetamide

A solution of 2-(5′-bromo-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide (50 mg, 0.090 mmol) in sulfuric acid (0.7 mL, 13.13 mmol) was treated portionwise with sodium azide (5.84 mg, 0.090 mmol) and then stirred at room temperature for 35 minutes. The mixture was poured into ice water and extracted with ethyl acetate. The extracts were washed with water, saturated sodium bicarbonate solution and brine, dried over Na₂SO₄, filtered, and concentrated to provide the title compound. ¹H NMR (400 MHz, DMSO-d₆) δ 10.05 (s, 1H), 8.62 (s, 1H), 7.46-7.02 (m, 7H), 5.27-4.03 (m, 7H), 1.33 (dt, J=6.9, 2.3 Hz, 3H). MS (ESI) m/e 571 (M+H)⁺.

### Example 639

### 2-[(1R)-5-({[3-(acetylamino)-2,2-dimethylpropyl]carbamoyl}amino)-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 346, substituting 2-[(1R)-5-{[(3-amino-2,2-dimethylpropyl)carbamoyl]amino}-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (400 MHz, DMSO-d₆) δ 8.43 (s, 1H), 7.51 (s, 1H), 7.42-7.04 (m, 7H), 6.08 (s, 1H), 5.26-5.09 (m, 1H), 4.85 (d, J=17.6 Hz, 1H), 4.73-4.51 (m, 2H), 4.39 (d, J=16.8 Hz, 1H), 3.22-2.45 (m, 8H), 1.87 (s, 3H), 1.39 (d, J=7.0 Hz, 3H), 0.85 (s, 6H). MS (ESI) m/e 650 (M+H)⁺.

### Example 640

### N-(4-fluorobenzyl)-2-(7′-{1-[2-(methylamino)-2-oxoethyl]-1H-pyrazol-4-yl}-2,2′,5-trioxo-2′,3′-dihydro-1H, 1′H-spiro[imidazolidine-4,4′-quinolin]-1-yl)-N-[(2S)-1,1,1-trifluoropropan-2-yl]acetamide

The title compound was prepared as described in EXAMPLE 295, substituting 2-(7′-bromo-2,2′,5-trioxo-2′,3′-dihydro-1H,1′H-spiro[imidazolidine-4,4′-quinolin]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 11.87 (s, 1H), 9.11-8.84 (m, 1H), 8.37-7.76 (m, 4H), 7.52-7.00 (m, 5H), 6.53-6.34 (m, 1H), 5.54-3.94 (m, 9H), 2.64 (d, J=4.4 Hz, 3H), 1.47-1.24 (m, 3H). MS (ESI) m/e 630 (M+H)⁺.

### Example 641

### 2-{7′-[1-(2-amino-2-oxoethyl)-1H-pyrazol-4-yl]-2,2′,5-trioxo-2′,3′-dihydro-1H,1′H-spiro[imidazolidine-4,4′-quinolin]-1-yl}-N-(4-fluorobenzyl)-N-[(2S)-1,1,1-trifluoropropan-2-yl]acetamide

The title compound was prepared as described in EXAMPLE 295, substituting 2-(7′-bromo-2,2′,5-trioxo-2′,3′-dihydro-1H,1′H-spiro[imidazolidine-4,4′-quinolin]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 11.87 (s, 1H), 9.10-8.87 (m, 1H), 8.32-6.99 (m, 10H), 6.52-6.31 (m, 1H), 5.58-3.90 (m, 9H), 1.45-1.23 (m, 3H). MS (ESI) m/e 616 (M+H)⁺.

### Example 643

### N-[(1S)-1-cyclopropyl-2,2,2-trifluoroethyl]-N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}acetamide

### Example 643A

### 2-(7′-amino-1′-methyl-2,5-dioxo-2′,3′-dihydro-1′H-spiro[imidazolidine-4,4′-quinolin]-1-yl)-N—((S)-1-cyclopropyl-2,2,2-trifluoroethyl)-N-(4-fluorobenzyl)acetamide

The title compound was prepared as described in EXAMPLE 296A-B, substituting 2-(7′-bromo-1′-methyl-2,5-dioxo-2′,3′-dihydro-1H,1′H-spiro[imidazolidine-4,4′-quinolin]-1-yl)-N-[(1S)-1-cyclopropyl-2,2,2-trifluoroethyl]-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide.

### Example 643B

### N-[(1S)-1-cyclopropyl-2,2,2-trifluoroethyl]-N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}acetamide

The title compound was prepared as described in EXAMPLE 282, substituting 2-(7′-amino-1′-methyl-2,5-dioxo-2′,3′-dihydro-1′H-spiro[imidazolidine-4,4′-quinolin]-1-yl)-N—((S)-1-cyclopropyl-2,2,2-trifluoroethyl)-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆) δ 8.73 (d, J=2.0 Hz, 1H), 8.35 (s, 1H), 7.43 (t, J=6.6 Hz, 1H), 7.31-7.01 (m, 2H), 6.98-6.40 (m, 3H), 5.92 (q, J=4.5 Hz, 1H), 5.10-4.02 (m, 5H), 3.39 (s, 1H), 3.16 (t, J=10.2 Hz, 1H), 2.82 (d, J=4.4 Hz, 2H), 2.59 (d, J=4.6 Hz, 2H), 2.11 (t, J=10.5 Hz, 2H), 1.22 (s, 2H), 0.91-0.55 (m, 3H), 0.19 (s, 2H). MS ESI⁺ 591 (M+H)⁺.

### Example 644

### 2-(5′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

### Example 644A

### 2-(5′-acetamido-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide

The title compound was prepared as described in EXAMPLE 281F, substituting N-(2,5-dioxo-2′,3′-dihydrospiro[imidazolidine-4,1′-inden]-5′-yl)acetamide for (R)-5-bromo-2,3-dihydrospiro[indene-1,5′-oxazolidine]-2′,4′-dione.

### Example 644B

### 2-(5′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

2-(5′-Acetamido-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide (70 mg, 0.134 mmol) in 0.5 mL tetrahydrofuran and 0.5 mL methanol was added hydrogen chloride (200 μl, 2.435 mmol). The mixture was heated at 60° C. overnight. The concentrated mixture was purified by reverse-phase HPLC to provide the title compound. ¹H NMR (501 MHz, DMSO-d₆) δ 8.81 (d, J=13.7 Hz, 1H), 7.44-6.82 (m, 7H), 5.42-3.98 (m, 5H), 3.00 (t, J=8.0 Hz, 2H), 2.68-2.52 (m, 1H), 2.37-2.13 (m, 1H), 1.32 (dd, J=31.1, 6.8 Hz, 3H). MS (ESI⁺) m/z 479 (M+H)⁺.

### Example 645

### 2,2,2-trifluoro-N-[1-(2-{(4-fluorobenzyl) [(2S)-1,1,1-trifluoropropan-2-yl]amino}-2-oxoethyl)-1′-methyl-2,5-dioxo-2′,3′-dihydro-1′H-spiro[imidazolidine-4,4′-quinolin]-7′-yl]acetamide

The title compound was isolated as a by-product from EXAMPLE 629B. ¹H NMR (400 MHz, DMSO-d₆) δ 11.08 (s, 1H), 8.86 (d, J=13.6 Hz, 1H), 7.39 (t, J=6.7 Hz, 1H), 7.23 (t, J=8.7 Hz, 2H), 7.12 (dt, J=13.6, 8.1 Hz, 2H), 7.01 (d, J=2.2 Hz, 1H), 6.97-6.87 (m, 1H), 5.38 (s, 1H), 4.95-4.63 (m, 2H), 4.47-4.29 (m, 1H), 3.47 (d, J=8.7 Hz, 1H), 3.23 (s, 2H), 2.17 (d, J=11.4 Hz, 1H), 2.01 (d, J=15.3 Hz, 1H), 1.41-1.13 (m, 5H), 0.84 (s, 1H). MS ESI⁺ 604 (M+H)⁺.

### Example 646

### N-[1-(2-{[(1S)-1-cyclopropylethyl](4-fluorobenzyl)amino}-2-oxoethyl)-1′-methyl-2,5-dioxo-2′,3′-dihydro-1′H-spiro[imidazolidine-4,4′-quinolin]-7′-yl]-2,2,2-trifluoroacetamide

The title compound was isolated as a by-product from EXAMPLE 526. ¹H NMR (400 MHz, DMSO-d₆) δ 11.08 (s, 1H), 8.82 (d, J=18.1 Hz, 1H), 7.44 (dd, J=8.6, 5.4 Hz, 1H), 7.28 (dd, J=8.4, 5.6 Hz, 1H), 7.25-7.13 (m, 2H), 7.09 (t, J=8.8 Hz, 1H), 7.02 (t, J=3.0 Hz, 1H), 6.96-6.87 (m, 1H), 4.73 (s, 1H), 4.60 (s, 1H), 4.47-4.22 (m, 1H), 4.22-4.10 (m, 1H), 3.79-3.41 (m, 1H), 3.35 (m, 1H), 3.26 (s, 2H), 2.16 (dd, J=11.7, 7.0 Hz, 1H), 2.01 (d, J=15.0 Hz, 1H), 1.31-1.03 (m, 4H), 1.00-0.79 (m, 2H), 0.50 (m, 1H), 0.41-0.08 (m, 3H). MS ESI⁺ 576 (M+H)⁺.

### Example 647

### N-(4-fluorobenzyl)-2-[(3′S,4S)-3′-hydroxy-2,5-dioxo-2′,3′-dihydro-1H-spiro[imidazolidine-4, 1′-inden]-1-yl]-N-[(2S)-1,1,1-trifluoropropan-2-yl]acetamide

The title compound was prepared as described in EXAMPLE 570A, substituting N-(4-fluorobenzyl)-N-[(2S)-1,1,1-trifluoropropan-2-yl]-2-[(4S)-2,3′,5-trioxo-2′,3′-dihydro-1H-spiro[imidazolidine-4,1′-inden]-1-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide. ¹H NMR (400 MHz, DMSO-d₆) δ 8.34 (s, 1H), 7.49-7.04 (m, 8H), 5.24 (dq, J=58.6, 7.6, 7.0 Hz, 2H), 4.94-4.21 (m, 5H), 2.64 (s, 1H), 2.44-2.26 (m, 1H), 1.35 (d, J=7.1 Hz, 3H). MS (ESI⁻) m/z 478 (M−H)⁻.

### Example 648

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-(pyridin-3-yl)acetamide

### Example 648A

### N-(4-fluorobenzyl)pyridin-3-amine

The title compound was prepared as described in EXAMPLE 283A, substituting pyridin-3-amine for (S)-1-cyclopropylethanamine and 4-fluorobenzaldehyde for benzaldehyde.

### Example 648B

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-(pyridin-3-yl)acetamide

A solution of (R)-2-(5-(3-methylureido)-2′,4′-dioxo-2,3-dihydrospiro[indene-1,5′-oxazolidin]-3′-yl)acetic acid (0.04 g, 0.120 mmol) in N,N-dimethylformamide (0.9 mL) was treated with N-(4-fluorobenzyl)pyridin-3-amine (0.027 g, 0.132 mmol), 2-(3H-[1,2,3]triazolo[4,5-b]pyridin-3-yl)-1,1,3,3-tetramethylisouronium hexafluorophosphate(V) (0.050 g, 0.132 mmol) and lastly, N-ethyl-N-isopropylpropan-2-amine (0.048 ml, 0.276 mmol), and the reaction mixture was stirred at room temperature for 16 hours. LCMS indicated little if any product and so triethylamine (0.033 ml, 0.240 mmol) and propylphosphonic anhydride (0.153 g, 0.240 mmol) (50% by wt soln in ethyl acetate) were added and the reaction mixture was stirred at room temperature for 72 hours. The reaction mixture was partitioned in ethyl acetate and water. The organic layer was washed with brine, dried over anhydrous sodium sulfate, filtered, and concentrated. Silica gel column chromatography provided the title compound. ¹H NMR (400 MHz, DMSO-d₆) δ ppm 2.38-2.49 (m, 1H), 2.56-2.71 (m, 4H), 2.92-3.20 (m, 2H), 3.99-4.20 (m, 2H), 4.70-5.22 (m, 2H), 6.07 (q, J=4.6 Hz, 1H), 7.19 (dt, J=47.0, 7.7 Hz, 6H), 7.46-7.59 (m, 2H), 7.81 (d, J=7.5 Hz, 1H), 8.47-8.64 (m, 2H), 8.70 (s, 1H). MS (ESI⁺) m/z 518 (M+H)⁺.

### Example 650

### N-(4-fluorobenzyl)-2-[3′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 514, substituting N-(4-fluorobenzyl)-2-[3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide. ¹H NMR (400 MHz, DMSO-d₆) δ 8.59 (s, 1H), 8.05 (s, 1H), 7.80 (s, 1H), 7.72-7.54 (m, 2H), 7.38-7.03 (m, 5H), 6.11 (ddd, J=57.8, 6.8, 4.5 Hz, 1H), 5.16 (p, J=7.8 Hz, 1H), 4.89-4.11 (m, 4H), 3.86 (s, 3H), 3.05 (dt, J=20.2, 6.8 Hz, 1H), 2.44-2.19 (m, 1H), 1.34 (d, J=6.8 Hz, 3H). MS (ESI⁺) m/z 562 (M+H)⁺.

### Example 655

### 2-(5′-bromo-3′-hydroxy-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

The title compound was prepared as described in EXAMPLE 570A, substituting 2-(5′-bromo-2,3′,5-trioxo-2′,3′-dihydrospiro[imidazolidine-4,1′-inden]-1-yl)-N-(4-fluorobenzyl)-N-((2S)-1,1,1-trifluoropropan-2-yl)acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide. ¹H NMR (400 MHz, DMSO-d₆) δ 8.39 (s, 1H), 7.56 (s, 1H), 7.51-6.97 (m, 7H), 5.31 (q, J=6.7 Hz, 1H), 5.15 (dt, J=21.2, 7.0 Hz, 2H), 4.87-4.21 (m, 4H), 2.64-2.32 (m, 2H), 1.47-1.26 (m, 3H). MS (ESI⁻) m/z 558 (M−H)⁻.

### Example 656

### 2-(5-{[(2-cyanoethyl)carbamoyl]amino}-6-fluoro-2′,4′-dioxo-2,3-dihydro-3′H-spiro[indene-1,5′-[1,3]oxazolidin]-3′-yl)-N-(4-fluorobenzyl)-N-[(2S)-1,1,1-trifluoropropan-2-yl]acetamide

The title compound was prepared as described in EXAMPLE 282, substituting 2-(5-amino-6-fluoro-2′,4′-dioxo-2,3-dihydro-3′H-spiro[indene-1,5′-[1,3]oxazolidin]-3′-yl)-N-(4-fluorobenzyl)-N-[(2S)-1,1,1-trifluoropropan-2-yl]acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and 3-aminopropanenitrile for 3,3-difluoroazetidine hydrochloride. ¹H NMR (400 MHz, DMSO-d₆) δ 8.65 (s, 1H), 8.18 (d, J=7.4 Hz, 1H), 7.39 (dd, J=8.4, 5.4 Hz, 1.3H), 7.34-7.21 (m, 3H), 7.13 (dt, J=13.6, 6.9 Hz, 0.7H), 7.04 (s, 1H), 5.49-5.34 (m, 0.7H), 5.13-4.70 (m, 2.3H), 4.52 (ddd, J=34.2, 22.9, 16.5 Hz, 1.3H), 4.20 (dd, J=17.1, 5.0 Hz, 0.7H), 3.37 (dd, J=12.2, 6.2 Hz, 2H), 3.20-3.06 (m, 1H), 3.02 (s, 1H), 2.67 (dt, J=15.2, 7.4 Hz, 3H), 2.61-2.52 (m, 1H), 1.47-1.29 (m, 3H). MS (ESI⁺) m/z 611 (M+NH4)⁺.

### Example 657

### N-(4-fluorobenzyl)-2-[(3′R,4S)-3′-fluoro-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 514, substituting N-(4-fluorobenzyl)-2-[(3′S,4S)-3′-hydroxy-2,5-dioxo-2′,3′-dihydro-1H-spiro[imidazolidine-4,1′-inden]-1-yl]-N-[(2S)-1,1,1-trifluoropropan-2-yl]acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide. ¹H NMR (400 MHz, DMSO-d₆) δ 8.61 (s, 1H), 7.55-6.93 (m, 8H), 6.11 (d, J=57.8 Hz, 1H), 5.15 (m, 1H), 4.88-4.12 (m, 4H), 3.04 (td, J=13.4, 6.8 Hz, 1H), 2.43-2.25 (m, 1H), 1.33 (d, J=7.0 Hz, 3H). MS (ESI⁺) m/z 482 (M+H)⁺.

### Example 658

### methyl [(1R)-3′-(2-{(4-fluorobenzyl) [(2S)-1,1,1-trifluoropropan-2-yl]amino}-2-oxoethyl)-2′,4′-dioxo-2,3-dihydrospiro[indene-1,5′-[1,3]oxazolidin]-5-yl]carbamate

The title compound was isolated as a by-product from EXAMPLE 659A. ¹H NMR (400 MHz, dimethylsulfoxide-d₆) δ ppm 1.34 (dd, J=18.9, 6.9 Hz, 3H), 2.41-2.73 (m, 2H), 2.92-3.24 (m, 2H), 3.66 (s, 3H), 4.13-4.64 (m, 2H), 4.66-5.53 (m, 3H), 6.98-7.64 (m, 7H), 9.82 (s, 1H). MS (ESI⁺) m/z 538.1 (M+H)⁺.

### Example 659

### 4-({[(1R)-3′-(2-{(4-fluorobenzyl) [(2S)-1, 1,1-trifluoropropan-2-yl]amino}-2-oxoethyl)-2′,4′-dioxo-2,3-dihydrospiro[indene-1,5′-[1,3]oxazolidin]-5-yl]carbamoyl}amino)butanoic acid

### Example 659A

### tert-butyl 4-(3-((R)-3′-(2-((4-fluorobenzyl)((S)-1,1,1-trifluoropropan-2-yl)amino)-2-oxoethyl)-2′,4′-dioxo-2,3-dihydrospiro[indene-1,5′-oxazolidin]-5-yl)ureido)butanoate

The title compound was prepared as described in EXAMPLE 282, substituting 2-[(1R)-5-amino-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and tert-butyl 4-aminobutanoate for 3,3-difluoroazetidine hydrochloride.

### Example 659B

### 4-({[(1R)-3′-(2-{(4-fluorobenzyl) [(2S)-1,1,1-trifluoropropan-2-yl]amino}-2-oxoethyl)-2′,4′-dioxo-2,3-dihydrospiro[indene-1,5′-[1,3]oxazolidin]-5-yl]carbamoyl}amino)butanoic acid

The title compound was prepared as described in EXAMPLE 302, substituting tert-butyl 4-(3-((R)-3′-(2-((4-fluorobenzyl)((S)-1,1,1-trifluoropropan-2-yl)amino)-2-oxoethyl)-2′,4′-dioxo-2,3-dihydrospiro[indene-1,5′-oxazolidin]-5-yl)ureido)butanoate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, dimethylsulfoxide-d₆) δ ppm 1.34 (dd, J=17.6, 6.9 Hz, 3H), 1.57-1.73 (m, 2H), 2.13-2.30 (m, 2H), 2.40-2.72 (m, 2H), 2.87-3.19 (m, 4H), 4.13-4.67 (m, 2H), 4.67-5.54 (m, 3H), 6.20-6.38 (m, 1H), 7.06-7.42 (m, 6H), 7.49-7.59 (m, 1H), 8.58-8.74 (m, 1H), 12.04 (s, 1H). MS (ESI⁺) m/z 609.2 (M+H)⁺.

### Example 660

### N²-acetyl-N-[(1R)-3′-(2-{(4-fluorobenzyl) [(2S)-1,1,1-trifluoropropan-2-yl]amino}-2-oxoethyl)-2′,4′-dioxo-2,3-dihydrospiro[indene-1,5′-[1,3]oxazolidin]-5-yl]-2-methylalaninamide

The title compound was prepared as described in EXAMPLE 346, substituting N-[(1R)-3′-(2-{(4-fluorobenzyl)[(2S)-1, 1,1-trifluoropropan-2-yl]amino}-2-oxoethyl)-2′,4′-dioxo-2,3-dihydrospiro[indene-1,5′-[1,3]oxazolidin]-5-yl]-2-methylalaninamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (400 MHz, dimethylsulfoxide-d₆) δ ppm 1.27-1.51 (m, 9H), 1.84 (s, 3H), 2.40-2.74 (m, 2H), 2.92-3.23 (m, 2H), 4.15-4.65 (m, 2H), 4.70-5.46 (m, 3H), 7.03-7.55 (m, 6H), 7.64-7.77 (m, 1H), 7.89-8.03 (m, 1H), 9.47 (s, 1H). MS (ESI⁺) m/z 607.2 (M+H)⁺.

### Example 661

### N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]oxazolidin]-5-yl]-2-methylalaninamide

### Example 661A

### tert-butyl (1-(((R)-3′-(2-((4-fluorobenzyl)((S)-1,1,1-trifluoropropan-2-yl)amino)-2-oxoethyl)-2′,4′-dioxo-2,3-dihydrospiro[indene-1,5′-oxazolidin]-5-yl)amino)-2-methyl-1-oxopropan-2-yl)carbamate

The title compound was prepared as described in EXAMPLE 306, substituting 2-((tert-butoxycarbonyl)amino)-2-methylpropanoic acid for cyclopropanecarboxylic acid and 2-[(1R)-5-amino-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 661B

### N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]oxazolidin]-5-yl]-2-methylalaninamide

The title compound was prepared as described in EXAMPLE 302, substituting tert-butyl (1-(((R)-3′-(2-((4-fluorobenzyl)((S)-1,1,1-trifluoropropan-2-yl)amino)-2-oxoethyl)-2′,4′-dioxo-2,3-dihydrospiro[indene-1,5′-oxazolidin]-5-yl)amino)-2-methyl-1-oxopropan-2-yl)carbamate for tert-butyl 3-[(1R)-3′-(2-{benzyl[(1 S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (500 MHz, dimethylsulfoxide-d₆) δ ppm 1.28 (d, J=3.4 Hz, 6H), 1.36 (dd, J=21.7, 7.0 Hz, 3H), 2.46-2.74 (m, 2H), 2.94-3.10 (m, 1H), 3.09-3.23 (m, 1H), 4.14-4.63 (m, 2H), 4.69-5.50 (m, 3H), 7.07-7.56 (m, 7H), 7.79-7.87 (m, 1H). MS (ESI⁺) m/z 565.2 (M+H)⁺.

### Example 667

### N-(4-fluorobenzyl)-2-{5′-[(methylcarbamoyl)amino]-2,3′,5-trioxo-2′,3′-dihydro-1H-spiro[imidazolidine-4,1′-inden]-1-yl}-N-[(2S)-1,1,1-trifluoropropan-2-yl]acetamide

The title compound was prepared as described in EXAMPLE 282, substituting 2-(5′-amino-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆) δ 8.48 (d, J=24.9 Hz, 2H), 7.98-7.22 (m, 6H), 7.10 (t, J=8.6 Hz, 2H), 5.91 (s, 1H), 5.16 (s, 1H), 4.88-4.13 (m, 4H), 3.02-2.80 (m, 2H), 2.67 (s, 4H), 1.34 (d, J=7.0 Hz, 3H). MS (ESI⁺) m/z 550 (M+H)⁺.

### Example 670

### 2-(5-{[(cyanomethyl)carbamoyl]amino}-6-fluoro-2′,4′-dioxo-2,3-dihydro-3′H-spiro[indene-1,5′-[1,3]oxazolidin]-3′-yl)-N-(4-fluorobenzyl)-N-[(2S)-1,1,1-trifluoropropan-2-yl]acetamide

The title compound was prepared as described in EXAMPLE 282, substituting 2-(5-amino-6-fluoro-2′,4′-dioxo-2,3-dihydro-3′H-spiro[indene-1,5′-[1,3]oxazolidin]-3′-yl)-N-(4-fluorobenzyl)-N-[(2S)-1,1,1-trifluoropropan-2-yl]acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and 2-aminoacetonitrile for 3,3-difluoroazetidine hydrochloride. ¹H NMR (400 MHz, DMSO-d₆) δ 8.84 (s, 1H), 8.10 (d, J=7.4 Hz, 1H), 7.45-7.00 (m, 5H), 5.37 (dd, J=15.5, 7.6 Hz, 0.7H), 5.12-4.64 (m, 2.7H), 4.64-4.35 (m, 1.3H), 4.17 (dd, J=13.8, 5.3 Hz, 2.3H), 3.16-2.90 (m, 2H), 2.72-2.49 (m, 2H), 1.39-1.26 (m, 3H). MS (ESI⁺) m/z 580 (M+H)⁺.

### Example 673

### 2-(5′-amino-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

### Example 673A

### 2-(5′-acetamido-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide

2-[5′-(Acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide (1 g, 1.921 mmol) in 30 mL acetone was cooled to −78° C. KMnO₄ (1.822 g, 11.53 mmol) and ferric chloride (0.935 g, 5.76 mmol) were added. The mixture was stirred at −78° C. for 1 hour, allowed to warm to room temperature, and stirred overnight. The resulting suspension was filtered with acetone washes and the filtrate was concentrated to dryness. Silica gel column chromatography provided the title compound.

### Example 673B

### 2-(5′-amino-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

To a solution of 2-(5′-acetamido-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide (200 mg, 0.374 mmol) in 2 mL tetrahydrofuran and 2 mL methanol was added hydrogen chloride (200 μl, 2.435 mmol). The mixture was heated at 60° C. for 6 hours, cooled to room temperature, diluted with 50 mL water and neutralized with solid sodium bicarbonate. The aqueous layer was extracted with ethyl acetate and the organic layers were washed with brine, dried over Na₂SO₄, filtered and concentrated. Silica gel column chromatography gave the title compound. ¹H NMR (400 MHz, DMSO-d₆) δ 8.35 (s, 1H), 7.47-6.74 (m, 7H), 5.40-4.96 (m, 3H), 4.88-4.09 (m, 4H), 3.09-2.61 (m, 2H), 1.43 (dd, J=78.7, 6.9 Hz, 3H). MS (ESI⁺) m/z 493 (M+H)⁺.

### Example 674

### tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]pyrrolidine-1-carboxylate

### Example 674A

### (R)-tert-butyl 3-((4-fluorobenzyl)amino)pyrrolidine-1-carboxylate

The title compound was prepared as described in EXAMPLE 283A, substituting (R)-tert-butyl 3-aminopyrrolidine-1-carboxylate for (S)-1-cyclopropylethanamine and 4-fluorobenzaldehyde for benzaldehyde.

### Example 674B

### tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]pyrrolidine-1-carboxylate

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and (R)-tert-butyl 3-((4-fluorobenzyl)amino)pyrrolidine-1-carboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆) δ 8.69 (d, J=5.4 Hz, 1H), 7.55 (s, 1H), 7.36-7.09 (m, 6H), 6.08 (s, 1H), 4.91-4.22 (m, 6H), 3.55 (t, J=8.6 Hz, 1H), 3.46-3.25 (m, 2H), 3.19-2.91 (m, 5H), 1.95 (d, J=28.3 Hz, 3H), 1.38 (d, J=17.3 Hz, 9H). MS (ESI⁺) m/z 510 (M-Boc+H)⁺.

### Example 675

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide

The title compound was prepared as described in EXAMPLE 302, substituting tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]pyrrolidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, methanol-d₄) δ 7.43 (dd, J=8.9, 5.7 Hz, 3H), 7.36 (s, 2H), 7.18 (t, J=8.7 Hz, 2H), 4.73 (s, 2H), 4.61 (dd, J=22.2, 12.9 Hz, 2H), 4.23 (dd, J=11.9, 8.1 Hz, 1H), 3.70-3.60 (m, 1H), 3.55 (dd, J=12.3, 4.0 Hz, 1H), 3.32 (ddd, J=7.8, 5.4, 4.9 Hz, 2H), 3.24-3.00 (m, 3H), 2.81-2.71 (m, 4H), 2.58-2.50 (m, 1H), 2.23 (dd, J=8.6, 5.1 Hz, 2H). MS (ESI⁺) m/z 510 (M+H)⁺.

### Example 677

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-1-(methylsulfonyl)pyrrolidin-3-yl]acetamide

To a suspension of N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide (40 mg, 0.069 mmol) in anhydrous dichloromethane (2.5 mL) was added triethylamine (0.038 mL, 0.275 mmol) and methanesulfonyl chloride (10.2 mg, 0.089 mmol). The solution was stirred at room temperature overnight. The solution was concentrated and purified directly by reverse-phase HPLC to give the title compound. ¹H NMR (400 MHz, methanol-d₄) δ 7.50 (s, 1H), 7.43-7.33 (m, 2H), 7.23 (t, J=8.3 Hz, 1H), 7.14 (t, J=8.6 Hz, 2H), 7.04 (t, J=7.9 Hz, 1H), 4.73 (dd, J=26.8, 12.8 Hz, 4H), 4.54-4.36 (m, 2H), 3.62-3.40 (m, 2H), 3.25-3.11 (m, 2H), 3.09-3.01 (m, 1H), 2.87 (s, 3H), 2.77-2.70 (m, 4H), 2.59-2.48 (m, 1H), 2.17-2.05 (m, 2H). MS (ESI⁺) m/z 588 (M+H)⁺.

### Example 678

### ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]pyrrolidin-1-yl}sulfonyl)carbamate

To a suspension of N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide (40 mg, 0.069 mmol) in anhydrous dichloromethane (2.5 mL) was added triethylamine (0.038 mL, 0.275 mmol) and ((4-(dimethylamino)pyridin-1-ium-1-yl)sulfonyl)(ethoxycarbonyl)amide (24.4 mg, 0.089 mmol). The solution was stirred at room temperature overnight. The solution was concentrated and purified directly by reverse-phase HPLC to give the title compound. ¹H NMR (500 MHz, methanol-d₄) δ 7.51 (d, J=5.9 Hz, 1H), 7.42-7.33 (m, 2H), 7.23 (d, J=7.8 Hz, 1H), 7.16 (dd, J=16.9, 8.3 Hz, 2H), 7.03 (t, J=8.5 Hz, 1H), 4.85 (s, 1H), 4.76-4.60 (m, 3H), 4.53-4.38 (m, 2H), 4.21-4.11 (m, 2H), 3.82 (t, J=8.8 Hz, 1H), 3.68-3.53 (m, 2H), 3.45-3.26 (m, 2H), 3.24-3.14 (m, 1H), 3.12-3.00 (m, 1H), 2.80-2.70 (m, 4H), 2.54 (ddd, J=14.1, 10.7, 6.6 Hz, 1H), 2.10 (dt, J=15.1, 7.0 Hz, 2H), 1.27 (dt, J=14.2, 7.1 Hz, 3H). MS (ESI⁺) m/z 661 (M+H)⁺.

### Example 679

### N-(4-fluorobenzyl)-2-[6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide

The title compound was prepared as described in EXAMPLE 295, substituting 2-(6-bromo-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆, 120° C.) δ ppm 1.29-1.41 (m, 3H), 3.72-3.89 (m, 4H), 3.96 (dd, J=14.5, 3.9 Hz, 1H), 4.29 (dd, J=16.4, 5.7 Hz, 1H), 4.42-4.71 (m, 2H), 4.82 (dd, J=17.6, 5.0 Hz, 1H), 5.09-5.28 (m, 1H), 7.06-7.18 (m, 2H), 7.26-7.39 (m, 2H), 7.63 (dd, J=8.1, 6.4 Hz, 1H), 7.91-8.00 (m, 3H), 8.22 (s, 1H), 8.92 (brs, 1H). MS (ESI⁺) m/z 594 (M+H)⁺.

### Example 680

### 2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

### Example 680A

### (S)-5′-bromo-2′,3′-dihydrospiro[imidazolidine-4,1′-indene]-2,5-dione and (R)-5′-bromo-2′,3′-dihydrospiro[imidazolidine-4,1′-indene]-2,5-dione

5′-Bromo-2′,3′-dihydrospiro[imidazolidine-4,1′-indene]-2,5-dione was purified by chiral SFC to give (R)-5′-bromo-2′,3′-dihydrospiro[imidazolidine-4,1′-indene]-2,5-dione (eluted second) and (S)-5′-bromo-2′,3′-dihydrospiro[imidazolidine-4,1′-indene]-2,5-dione (eluted first). The absolute configuration was assigned by single crystal x-ray diffraction.

### Example 680B

### 2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 281F, substituting (S)-5′-bromo-2′,3′-dihydrospiro[imidazolidine-4,1′-indene]-2,5-dione for (R)-5-bromo-2,3-dihydrospiro[indene-1,5′-oxazolidine]-2′,4′-dione. ¹H NMR (400 MHz, DMSO-d₆) δ 8.16 (s, 1H), 7.70 (s, 1H), 7.31-6.78 (m, 6H), 4.90 (h, J=7.5 Hz, 1H), 4.69-3.78 (m, 4H), 2.81 (t, J=7.2 Hz, 2H), 2.32 (dt, J=13.3, 6.6 Hz, 1H), 2.09-1.83 (m, 1H), 1.23-0.90 (m, 3H). MS (ESI⁻) m/z 540 (M−H)⁻.

### Example 681

### N-(4-fluorobenzyl)-2-{3′-hydroxy-5′-[1-(2-methylpropyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide

The title compound was prepared as described in EXAMPLE 295, substituting 2-(5′-bromo-3′-hydroxy-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-isobutyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.34 (s, 1H), 8.00 (s, 1H), 7.80-7.01 (m, 8H), 5.31 (q, J=6.6 Hz, 1H), 5.19 (p, J=7.9 Hz, 1H), 4.95-4.22 (m, 5H), 3.91 (dd, J=19.6, 6.9 Hz, 3H), 2.60-2.30 (m, 1H), 2.17 (tt, J=13.1, 6.6 Hz, 1H), 1.35 (d, J=7.0 Hz, 3H), 0.87 (dd, J=17.9, 6.7 Hz, 6H). MS (ESI⁺) m/z 602 (M+H)⁺.

### Example 682

### N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 295, substituting 2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.33 (s, 1H), 7.95 (s, 1H), 7.72 (s, 1H), 7.46-7.01 (m, 7H), 5.17 (p, J=7.7 Hz, 1H), 4.97-4.15 (m, 4H), 3.84 (s, 3H), 3.06 (dd, J=13.7, 6.4 Hz, 2H), 2.59 (dd, J=13.3, 6.7 Hz, 1H), 2.25 (dt, J=13.4, 7.7 Hz, 1H), 1.34 (d, J=7.1 Hz, 3H). MS (ESI⁺) m/z 544 (M+H)⁺.

### Example 683

### N-(4-fluorobenzyl)-2-[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 295, substituting 2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.36 (s, 1H), 8.02 (s, 1H), 7.79 (s, 1H), 7.53-7.02 (m, 8H), 5.17 (p, J=7.8 Hz, 1H), 4.88-4.13 (m, 6H), 3.15-3.00 (m, 2H), 2.65 (d, J=4.5 Hz, 3H), 2.59 (dd, J=13.3, 6.7 Hz, 1H), 2.40-2.11 (m, 1H), 1.34 (d, J=7.0 Hz, 3H). MS (ESI⁺) m/z 601 (M+H)⁺.

### Example 685

### 2-[(4 S)-5′-bromo-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

### Example 685A

### (S)-5′-bromospiro[imidazolidine-4,1′-indene]-2,3′,5(2′H)-trione

A mixture of (S)-5′-bromo-2′,3′-dihydrospiro[imidazolidine-4,1′-indene]-2,5-dione (2 g, 7.11 mmol), sodium 2-iodobenzenesulfonate (0.109 g, 0.356 mmol), tetrabutylammonium hydrogensulfate (0.483 g, 1.423 mmol) and potassium peroxysulfate (13.12 g, 21.34 mmol) in 75 mL acetonitrile was heated at 65° C. for 20 hours. The mixture was cooled and an additional 2 g of potassium peroxysulfate was added. The mixture was heated at 65° C. for an additional 3 hours and then filtered with acetone washes. The filtrate was concentrated to dryness, triturated with acetone, refiltered and the filtrate was taken to dryness. The crude product was suspended in 15 mL ethyl acetate, stirred at room temperature for 2 hours and filtered to give the first crop of title compound. The filtrate was purified by silical gel column chromatography to give additional title compound.

### Example 685B

### 2-[(4S)-5′-bromo-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 281F, substituting (S)-5′-bromospiro[imidazolidine-4,1′-indene]-2,3′,5(2′H)-trione for (R)-5-bromo-2,3-dihydrospiro[indene-1,5′-oxazolidine]-2′,4′-dione. ¹H NMR (400 MHz, DMSO-d₆) δ 8.56 (s, 1H), 7.94 (d, J=7.6 Hz, 1H), 7.84 (s, 1H), 7.62 (d, J=8.2 Hz, 1H), 7.42-6.99 (m, 4H), 5.16 (p, J=7.8 Hz, 1H), 4.88-4.19 (m, 4H), 3.28-2.91 (m, 2H), 1.34 (d, J=7.0 Hz, 3H). MS (ESI⁻) m/z 556 (M−H)⁻.

### Example 686

### 2-(5′-bromo-3′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

The title compound was prepared as described in EXAMPLE 514, substituting 2-(5′-bromo-3′-hydroxy-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide. ¹H NMR (400 MHz, DMSO-d₆) δ 8.65 (s, 1H), 7.80-7.03 (m, 7H), 6.10 (dt, J=57.0, 5.8 Hz, 1H), 5.15 (p, J=7.8 Hz, 1H), 4.87-4.15 (m, 4H), 3.05 (td, J=13.4, 6.9 Hz, 1H), 2.45-2.16 (m, 1H), 1.33 (d, J=6.9 Hz, 3H). MS (ESI⁻) m/z 560 (M−H)⁻.

### Example 687

### 5-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]oxazolidin]-5-yl]carbamoyl}amino)pentanoic acid

The title compound was prepared as described in EXAMPLE 282, substituting 2-[(1R)-5-amino-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and 5-aminopentanoic acid for 3,3-difluoroazetidine hydrochloride. ¹H NMR (400 MHz, dimethylsulfoxide-d₆) δ ppm 1.26-1.62 (m, 7H), 2.21 (td, J=7.2, 1.8 Hz, 2H), 2.39-2.71 (m, 2H), 2.86-3.22 (m, 4H), 4.13-4.62 (m, 2H), 4.66-5.50 (m, 3H), 6.20 (t, J=5.8 Hz, 1H), 7.05-7.60 (m, 7H), 8.46-8.68 (m, 1H), 11.99 (s, 1H). MS (ESI⁺) m/z 623.1 (M+H)⁺.

### Example 688

### N-[(1S)-1-cyclopropyl-2,2,2-trifluoroethyl]-N-(4-fluorobenzyl)-2-[4-fluoro-5-(1-methyl-1H-pyrazol-4-yl)-2′,4′-dioxo-2,3-dihydro-3′H-spiro[indene-1,5′-[1,3]oxazolidin]-3′-yl]acetamide

The title compound was prepared as described in EXAMPLE 295, substituting 2-(5-bromo-4-fluoro-2′,4′-dioxo-2,3-dihydro-3′H-spiro[indene-1,5′-[1,3]oxazolidin]-3′-yl)-N-[(1S)-1-cyclopropyl-2,2,2-trifluoroethyl]-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.03 (s, 1H), 7.80 (s, 1H), 7.67-6.94 (m, 6H), 4.97-4.16 (m, 5H), 3.30-3.00 (m, 2H), 2.79-2.52 (m, 2H), 1.25 (s, 1H), 0.74 (dd, J=22.6, 14.4 Hz, 2H), 0.35 (d, J=38.5 Hz, 2H). MS (ESI⁺) m/z 589 (M+H)⁺.

### Example 689

### N-(4-fluorobenzyl)-2-[6-fluoro-5-(1-methyl-1H-pyrazol-4-yl)-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-(5-bromo-6-fluoro-2′,4′-dioxo-2,3-dihydro-3′H-spiro[indene-1,5′-[1,3]oxazolidin]-3′-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.02 (s, 1H), 7.80 (s, 1H), 7.64 (d, J=6.9 Hz, 1H), 7.37-7.29 (m, 2H), 7.26 (d, J=10.7 Hz, 1H), 7.12 (t, J=8.6 Hz, 2H), 5.18 (s, 1H), 4.83 (d, J=17.4 Hz, 1H), 4.62 (d, J=18.0 Hz, 2H), 4.36 (d, J=16.8 Hz, 1H), 3.87 (s, 3H), 3.23-2.97 (m, 2H), 2.78-2.65 (m, 1H), 2.59-2.49 (m, 1H), 1.37 (d, J=7.1 Hz, 3H). MS (ESI⁺) m/z 563 (M+H)⁺.

### Example 690

### 2-{(4S)-5′-[1-(2-amino-2-oxoethyl)-1H-pyrazol-4-yl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide

The title compound was prepared as described in EXAMPLE 295, substituting 2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.34 (s, 1H), 8.02 (s, 1H), 7.79 (s, 1H), 7.52-7.07 (m, 7H), 6.86 (s, 2H), 5.17 (p, J=7.8 Hz, 1H), 4.89-4.12 (m, 6H), 3.06 (t, J=7.2 Hz, 2H), 2.59 (dd, J=13.4, 6.7 Hz, 1H), 2.37-1.97 (m, 1H), 1.34 (d, J=7.0 Hz, 3H). MS (ESI⁺) m/z 587 (M+H)⁺.

### Example 691

### N-[(1S)-1-cyclopropylethyl]-N-(4-fluorobenzyl)-2-[1′-methyl-7′-(1-methyl-1H-pyrazol-4-yl)-2,5-dioxo-2′,3′-dihydro-1H,1′H-spiro[imidazolidine-4,4′-quinolin]-1-yl]acetamide

The title compound was prepared as described in EXAMPLE 295, substituting 2-(7′-bromo-1′-methyl-2,5-dioxo-2′,3′-dihydro-1H,1′H-spiro[imidazolidine-4,4′-quinolin]-1-yl)-N-[(1S)-1-cyclopropylethyl]-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.78 (d, J=17.7 Hz, 1H), 8.09 (d, J=3.6 Hz, 1H), 7.82 (d, J=4.0 Hz, 1H), 7.43 (dd, J=8.3, 5.5 Hz, 1H), 7.26 (s, 1H), 7.22-7.04 (m, 3H), 6.81 (d, J=18.3 Hz, 2H), 4.71 (s, 1H), 4.59 (s, 1H), 4.45-4.21 (m, 2H), 4.17 (dd, J=13.4, 3.8 Hz, 1H), 3.82 (d, J=2.5 Hz, 3H), 3.70 (q, J=7.4 Hz, 1H), 3.42 (d, J=7.9 Hz, 1H), 3.21 (d, J=8.8 Hz, 1H), 2.93 (d, J=2.7 Hz, 3H), 2.14 (s, 1H), 2.00 (s, 1H), 1.22 (s, 2H), 1.14 (d, J=30.3 Hz, 2H), 0.92 (s, 1H), 0.83 (d, J=7.3 Hz, 2H), 0.47 (s, 1H). MS (ESI⁺) m/z 545 (M+H)⁺.

### Example 692

### N-(4-fluorobenzyl)-2-[7′-(1-methyl-1H-pyrazol-4-yl)-2,2′,5-trioxo-2′,3′-dihydro-1H,1′H-spiro[imidazolidine-4,4′-quinolin]-1-yl]-N-[(2S)-1,1,1-trifluoropropan-2-yl]acetamide

The title compound was prepared as described in EXAMPLE 295, substituting 2-(7′-bromo-2,2′,5-trioxo-2′,3′-dihydro-1H,1′H-spiro[imidazolidine-4,4′-quinolin]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 11.86 (s, 1H), 9.06-8.85 (m, 1H), 8.33-7.08 (m, 8H), 6.43 (d, J=15.0 Hz, 1H), 5.61-3.95 (m, 7H), 3.88 (d, J=8.6 Hz, 3H), 1.35 (dd, J=21.7, 6.6 Hz, 3H). MS (ESI) m/e 573 (M+H)⁺.

### Example 693

### N-(4-fluorobenzyl)-2-[(4S)-5′-(1-methyl-1H-pyrazol-4-yl)-2,3′,5-trioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 295, substituting 2-[(4S)-5′-bromo-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.51 (s, 1H), 8.13 (s, 1H), 7.99-7.55 (m, 4H), 7.43-7.21 (m, 2H), 7.10 (t, J=8.7 Hz, 2H), 5.17 (p, J=7.8 Hz, 1H), 4.95-4.16 (m, 4H), 3.86 (s, 3H), 2.99 (dd, J=63.4, 18.4 Hz, 2H), 1.35 (d, J=7.0 Hz, 3H). MS (ESI⁺) m/z 558.0 (M+H)⁺.

### Example 694

### N-[(3R)-1-acetylpyrrolidin-3-yl]-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 346, substituting N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (501 MHz, methanol-d₄) δ 7.51 (s, 1H), 7.42-7.33 (m, 2H), 7.26-7.11 (m, 3H), 7.08-6.98 (m, 1H), 4.98-4.91 (m, 1H), 4.74 (dt, J=28.0, 15.4 Hz, 3H), 4.55-4.39 (m, 2H), 3.85-3.70 (m, 1H), 3.68-3.55 (m, 1H), 3.50-3.38 (m, 1H), 3.38-3.25 (m, 1H), 3.24-3.14 (m, 1H), 3.12-3.00 (m, 1H), 2.80-2.70 (m, 4H), 2.59-2.48 (m, 1H), 2.23-2.01 (m, 2H), 1.99 (d, J=5.7 Hz, 2H), 1.95 (s, 1H). MS (ESI⁺) m/z 574 (M+Na)⁺.

### Example 695

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-1-(2-sulfamoylethyl)pyrrolidin-3-yl]acetamide

To a solution of N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide (40 mg, 0.069 mmol) in N,N-dimethylformamide (2 mL) was added triethylamine (0.038 mL, 0.275 mmol) and ethenesulfonamide (8.83 mg, 0.082 mmol). This reaction mixture was heated at 70° C. overnight, diluted with water and purified directly by reverse-phase HPLC to afford the title compound. ¹H NMR (501 MHz, methanol-d₄) δ 7.50-7.41 (m, 4H), 7.38-7.30 (m, 1H), 7.18 (t, J=8.7 Hz, 2H), 4.75-4.69 (m, 1H), 4.67-4.58 (m, 2H), 4.25-4.18 (m, 1H), 4.08-3.92 (m, 2H), 3.79-3.71 (m, 1H), 3.68-3.51 (m, 4H), 3.34-3.27 (m, 2H), 3.19 (dt, J=15.5, 7.6 Hz, 1H), 3.10-3.02 (m, 1H), 2.83-2.76 (m, 4H), 2.60-2.48 (m, 1H), 2.41-2.17 (m, 2H). MS (ESI⁺) m/z 617 (M+H)⁺.

### Example 698

### N-(4-fluorobenzyl)-2-[(3′R,4S)-3′-hydroxy-2,5-dioxo-2′,3′-dihydro-1H-spiro[imidazolidine-4,1′-inden]-1-yl]-N-[(2S)-1,1,1-trifluoropropan-2-yl]acetamide

(S)-2-Methyl-CBS-oxazaborolidine (8.71 mg, 0.031 mmol) in 1 mL tetrahydrofuran was cooled to 0° C. Borane dimethylsulfide complex (79 μl, 0.157 mmol) solution was added dropwise and the mixture was stirred at 0° C. for 30 minutes. N-(4-Fluorobenzyl)-N—((S)-1,1,1-trifluoropropan-2-yl)-2-((S)-2,3′,5-trioxo-2′,3′-dihydrospiro[imidazolidine-4,1′-inden]-1-yl)acetamide (50 mg, 0.105 mmol) in 0.5 mL tetrahydrofuran was added dropwise at 0° C. The reaction was stirred at 0° C. for 2 hours. The mixture was concentrated and silica gel column chromatography gave the title compound. ¹H NMR (400 MHz, DMSO-d₆) δ 7.51-6.96 (m, 9H), 5.23 (t, J=6.8 Hz, 1H), 5.15 (p, J=7.7 Hz, 1H), 4.86-3.20 (m, 5H), 2.83 (d, J=6.0 Hz, 1H), 2.24-1.97 (m, 1H), 1.33 (d, J=7.0 Hz, 3H). MS (ESI⁺) m/z 480 (M+H)⁺.

### Example 699

### N-(4-fluorobenzyl)-2-[(3′S,4S)-3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 570A, substituting N-(4-fluorobenzyl)-2-[(4S)-5′-(1-methyl-1H-pyrazol-4-yl)-2,3′,5-trioxo-2′,3′-dihydro-1H-spiro[imidazolidine-4,1′-inden]-1-yl]-N-[(2S)-1,1,1-trifluoropropan-2-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide. ¹H NMR (400 MHz, DMSO-d₆) δ 8.34 (s, 1H), 7.98 (s, 1H), 7.74 (s, 1H), 7.63-7.04 (m, 7H), 5.31 (q, J=6.6 Hz, 1H), 5.19 (p, J=7.9 Hz, 1H), 5.01-4.21 (m, 5H), 3.68 (s, 3H), 2.52 (d, J=6.7 Hz, 1H), 2.39 (dd, J=13.5, 6.1 Hz, 1H), 1.35 (d, J=7.0 Hz, 3H). MS (ESI⁺) m/z 560 (M+H)⁺.

### Example 700

### N-(4-fluorobenzyl)-2-[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 514, substituting N-(4-fluorobenzyl)-2-[(3′S,4S)-3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide. ¹H NMR (400 MHz, DMSO-d₆) δ 8.04 (s, 1H), 7.79 (s, 1H), 7.71-7.50 (m, 2H), 7.32 (dd, J=8.3, 5.4 Hz, 3H), 7.10 (t, J=8.7 Hz, 2H), 6.22-5.99 (m, 1H), 5.15 (p, J=7.7 Hz, 1H), 4.91-3.98 (m, 5H), 3.86 (s, 3H), 3.18-3.09 (m, 1H), 2.48-2.29 (m, 1H), 1.34 (d, J=7.0 Hz, 3H). MS (ESI⁻) m/z 560 (M−H)⁻.

### Example 701

### N-(4-fluorobenzyl)-2-[(4S)-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 295, substituting 2-[(4S)-5′-bromo-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.53 (s, 1H), 8.20 (s, 1H), 8.10-7.87 (m, 2H), 7.83 (s, 1H), 7.64 (d, J=8.1 Hz, 1H), 7.50 (dq, J=13.6, 7.4 Hz, 1H), 7.33 (dd, J=8.4, 5.3 Hz, 2H), 7.10 (t, J=8.7 Hz, 2H), 5.17 (p, J=7.8 Hz, 1H), 4.84-4.12 (m, 6H), 3.08 (d, J=18.4 Hz, 1H), 2.92 (d, J=18.4 Hz, 1H), 2.66 (d, J=4.7 Hz, 3H), 1.35 (d, J=7.0 Hz, 3H). MS (ESI⁺) m/z 615 (M+H)⁺.

### Example 702

### N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]oxazolidin]-5-yl]-3-(methylsulfonyl)tetrahydropyrimidine-1 (2H)-carboxamide

Methanesulfonyl chloride (7.0 μl, 0.090 mmol) was added to a 0° C. solution of N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]oxazolidin]-5-yl]tetrahydropyrimidine-1(2H)-carboxamide (60 mg, 0.085 mmol), triethylamine (36 μl, 0.258 mmol) and dichloromethane (1.9 ml). The reaction mixture was stirred at 0° C. for 10 minutes and at room temperature for 20 hours. Water was added and the separated aqueous layer was extracted with ethyl acetate. The combined extracts were washed with water and brine, dried with MgSO₄, filtered and concentrated. The residue was purified by silica gel column chromatography to afford the title compound. ¹H NMR (501 MHz, DMSO-d₆) δ 8.95 (s, 1H), 7.53 (s, 1H), 7.41-7.20 (m, 4H), 7.12 (t, J=8.7 Hz, 1H), 5.46-5.33 (m, 1H), 4.93 (d, J=17.6 Hz, 3H), 4.73 (d, J=18.7 Hz, 1H), 4.54 (t, J=16.5 Hz, 1H), 4.19 (d, J=17.1 Hz, 1H), 3.62 (s, 2H), 3.45 (t, J=5.7 Hz, 2H), 3.11 (dt, J=14.9, 7.6 Hz, 1H), 3.01 (s, 1H), 2.92 (d, J=2.2 Hz, 2H), 2.63 (dq, J=14.9, 7.8, 7.3 Hz, 1H), 1.67 (s, 2H), 1.34 (dd, J=17.9, 6.9 Hz, 2H). MS ESI⁺ 670 (M+H)⁺.

### Example 703

### N-(4-fluorobenzyl)-2-[(3′S,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 570A, substituting N-(4-fluorobenzyl)-2-[(4S)-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl]-N-[(2S)-1,1,1-trifluoropropan-2-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide. ¹H NMR (400 MHz, DMSO-d₆) δ 8.34 (s, 1H), 8.04 (s, 1H), 7.80 (s, 1H), 7.59-7.38 (m, 3H), 7.38-7.20 (m, 3H), 7.11 (t, J=8.6 Hz, 2H), 5.32 (q, J=6.7 Hz, 1H), 5.17 (q, J=7.7 Hz, 1H), 4.95-4.71 (m, 4H), 4.67-4.42 (m, 2H), 4.28 (d, J=16.6 Hz, 1H), 2.65 (d, J=4.7 Hz, 3H), 2.54 (d, J=7.5 Hz, 1H), 2.39 (d, J=7.5 Hz, 1H), 1.35 (d, J=7.0 Hz, 3H). MS (ESI⁺) m/z 617 (M+H)⁺.

### Example 704

### N-(3,5-difluorobenzyl)-2-[(4S)-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl]-N-[(2S)-1,1,1-trifluoropropan-2-yl]acetamide

### Example 704A

### (S)-2-bromo-N-(3,5-difluorobenzyl)-N-(1,1,1-trifluoropropan-2-yl)acetamide

The title compound was prepared as described in EXAMPLE 281A-B, substituting 1-(bromomethyl)-3,5-difluorobenzene for 1-(bromomethyl)-4-fluorobenzene in EXAMPLE 281A.

### Example 704B

### 2-((S)-5′-bromo-2,3′,5-trioxo-2′,3′-dihydrospiro[imidazolidine-4,1′-inden]-1-yl)-N-(3,5-difluorobenzyl)-N—((S)-1,1,1-trifluoropropan-2-yl)acetamide

The title compound was prepared as described in EXAMPLE 281F, substituting (S)-5′-bromospiro[imidazolidine-4,1′-indene]-2,3′,5(2′H)-trione for (R)-5-bromo-2,3-dihydrospiro[indene-1,5′-oxazolidine]-2′,4′-dione and (S)-2-bromo-N-(3,5-difluorobenzyl)-N-(1,1,1-trifluoropropan-2-yl)acetamide for (S)-2-bromo-N-(4-fluorobenzyl)-N-(1,1,1-trifluoropropan-2-yl)acetamide.

### Example 704C

### N-(3,5-difluorobenzyl)-2-[(4S)-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 295, substituting 2-((S)-5′-bromo-2,3′,5-trioxo-2′,3′-dihydrospiro[imidazolidine-4,1′-inden]-1-yl)-N-(3,5-difluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.54 (s, 1H), 8.19 (s, 1H), 8.01-7.38 (m, 5H), 6.95 (dd, J=13.6, 8.3 Hz, 3H), 5.25-4.23 (m, 7H), 3.00 (dd, J=62.6, 18.4 Hz, 2H), 2.66 (d, J=4.7 Hz, 3H), 1.37 (d, J=7.0 Hz, 3H). MS (ESI) m/e 633 (M+H)⁺.

### Example 705

### N-(3,5-difluorobenzyl)-2-[(3′S,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 570A, substituting N-(3,5-difluorobenzyl)-2-[(4S)-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl]-N-[(2S)-1,1,1-trifluoropropan-2-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide. ¹H NMR (400 MHz, DMSO-d₆) δ 8.36 (s, 1H), 8.04 (s, 1H), 7.80 (s, 1H), 7.61-6.85 (m, 7H), 5.40-4.20 (m, 9H), 2.66 (d, J=4.7 Hz, 3H), 2.58-2.29 (m, 2H), 1.37 (d, J=7.0 Hz, 3H). MS (ESI) m/e 635 (M+H)⁺.

### Example 706

### N-(3,5-difluorobenzyl)-2-[(3′R,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 514, substituting N-(3,5-difluorobenzyl)-2-[(3′S,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2 S)-1,1,1-trifluoropropan-2-yl]acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide ¹H NMR (400 MHz, DMSO-d₆) δ 8.61 (s, 1H), 8.10 (s, 1H), 7.85 (s, 1H), 7.71-6.87 (m, 7H), 6.19 (dd, J=6.8, 4.6 Hz, 1H), 5.24-4.19 (m, 7H), 3.12-2.99 (m, 1H), 2.66 (d, J=4.7 Hz, 3H), 2.47-2.28 (m, 1H), 1.36 (d, J=7.0 Hz, 3H). MS (ESI) m/e 637 (M+H)⁺.

### Example 707

### N²-acetyl-N-[(1R)-3′-(2-{(4-fluorobenzyl)[(2S)-1, 1,1-trifluoropropan-2-yl]amino}-2-oxoethyl)-2′,4′-dioxo-2,3-dihydrospiro[indene-1,5′-[1,3]oxazolidin]-5-yl]glycinamide

### Example 707A

### tert-butyl (2-(((R)-3′-(2-((4-fluorobenzyl)((S)-1,1,1-trifluoropropan-2-yl)amino)-2-oxoethyl)-2′,4′-dioxo-2,3-dihydrospiro[indene-1,5′-oxazolidin]-5-yl)amino)-2-oxoethyl)carbamate

The title compound was prepared as described in EXAMPLE 306, substituting 2-((tert-butoxycarbonyl)amino)acetic acid for cyclopropanecarboxylic acid and 2-[(1R)-5-amino-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 707B

### 2-((R)-5-(2-aminoacetamido)-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide

The title compound was prepared as described in EXAMPLE 302, substituting tert-butyl (2-(((R)-3′-(2-((4-fluorobenzyl)((S)-1,1,1-trifluoropropan-2-yl)amino)-2-oxoethyl)-2′,4′-dioxo-2,3-dihydrospiro[indene-1,5′-oxazolidin]-5-yl)amino)-2-oxoethyl)carbamate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 707C

### N²-acetyl-N-[(1R)-3′-(2-{(4-fluorobenzyl) [(2S)-1,1,1-trifluoropropan-2-yl]amino}-2-oxoethyl)-2′,4′-dioxo-2,3-dihydrospiro[indene-1,5′-[1,3]oxazolidin]-5-yl]glycinamide

The title compound was prepared as described in EXAMPLE 346, substituting 2-((R)-5-(2-aminoacetamido)-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (400 MHz, dimethylsulfoxide-d₆) δ ppm 1.33 (dd, J=17.2, 6.9 Hz, 3H), 1.85 (s, 3H), 2.41-2.71 (m, 2H), 2.93-3.22 (m, 2H), 3.85 (dd, J=5.9, 2.4 Hz, 2H), 4.15-4.59 (m, 2H), 4.66-5.01 (m, 2H), 5.01-5.47 (m, 1H), 7.01-7.48 (m, 6H), 7.63-7.74 (m, 1H), 8.11-8.26 (m, 1H), 10.09 (s, 1H). MS (ESI⁺⁾ m/z 579.2 (M+H)⁺.

### Example 711

### 2-[(3′S,4S)-5′-bromo-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 570A, substituting 2-[(4S)-5′-bromo-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide. ¹H NMR (400 MHz, DMSO-d₆) δ 8.40 (s, 1H), 7.56 (s, 1H), 7.48 (d, J=8.2 Hz, 1H), 7.33 (t, J=6.9 Hz, 2H), 7.23 (d, J=8.2 Hz, 1H), 7.11 (t, J=8.6 Hz, 2H), 5.31 (q, J=6.6 Hz, 1H), 5.16 (dt, J=23.5, 7.3 Hz, 2H), 4.90-4.18 (m, 4H), 2.64-2.50 (m, 1H), 2.37 (dd, J=13.5, 6.5 Hz, 1H), 1.35 (d, J=7.1 Hz, 3H). MS (ESI⁻) m/z 556 (M−H)⁻.

### Example 713

### N-(4-fluorobenzyl)-N-(4-fluorophenyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 648B, substituting 4-fluoro-N-(4-fluorobenzyl)aniline for N-(4-fluorobenzyl)pyridin-3-amine. ¹H NMR (400 MHz, DMSO-d₆, 120° C.) δ ppm 8.27 (brs, 1H), 7.48 (s, 1H), 7.34-7.15 (m, 8H), 7.05 (t, J=8.7 Hz, 2H), 5.84 (brs, 1H), 4.88 (s, 2H), 4.16-4.02 (m, 2H), 3.20-2.91 (m, 2H), 2.73-2.59 (m, 4H), 2.49-2.39 (m, 1H). MS (ESI⁻) m/z 535 (M−H)⁻.

### Example 714

### N-(4-fluorobenzyl)-N-(2-fluorophenyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 648B, substituting 2-fluoro-N-(4-fluorobenzyl)aniline for N-(4-fluorobenzyl)pyridin-3-amine. ¹H NMR (400 MHz, DMSO-d₆, 120° C.) δ ppm 8.26 (brs, 1H), 7.52-7.38 (m, 2H), 7.38-7.14 (m, 7H), 7.14-6.95 (m, 2H), 5.84 (brs, 1H), 4.88 (brs, 2H), 4.27-3.95 (m, 2H), 3.18-2.92 (m, 2H), 2.73-2.57 (m, 4H), 2.48-2.39 (m, 1H). MS (ESI⁻) m/z 535 (M−H)⁻.

### Example 715

### N-(4-fluorobenzyl)-2-[(3′R,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 514, substituting N-(4-fluorobenzyl)-2-[(3′S,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2 S)-1,1,1-trifluoropropan-2-yl]acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide. ¹H NMR (400 MHz, DMSO-d₆) δ 8.42 (s, 1H), 7.93 (s, 1H), 7.68 (s, 1H), 7.61-7.40 (m, 2H), 7.30 (s, 1H), 7.15 (dd, J=12.3, 6.8 Hz, 3H), 6.92 (t, J=8.6 Hz, 2H), 5.95 (dt, J=57.7, 5.7 Hz, 1H), 4.97 (h, J=7.8 Hz, 1H), 4.67-4.00 (m, 6H), 2.88 (td, J=13.4, 6.8 Hz, 1H), 2.48 (d, J=4.4 Hz, 3H), 2.26-2.05 (m, 1H), 1.17 (d, J=7.0 Hz, 3H). MS (ESI⁺) m/z 619 (M+H)⁺.

### Example 716

### 2-[(3′R,4S)-5′-bromo-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 698, substituting 2-[(4S)-5′-bromo-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for N-(4-fluorobenzyl)-N—((S)-1,1,1-trifluoropropan-2-yl)-2-((S)-2,3′,5-trioxo-2′,3′-dihydrospiro[imidazolidine-4,1′-inden]-1-yl)acetamide. ¹H NMR (400 MHz, DMSO-d₆) δ 8.43 (s, 1H), 7.58 (s, 1H), 7.48 (d, J=8.3 Hz, 1H), 7.31 (dd, J=8.3, 5.3 Hz, 2H), 7.26-6.96 (m, 3H), 5.35 (t, J=4.7 Hz, 1H), 5.17 (dq, J=39.0, 7.7, 7.2 Hz, 2H), 4.86-4.00 (m, 4H), 2.85 (d, J=7.1 Hz, 1H), 2.11 (dd, J=13.2, 7.0 Hz, 1H), 1.33 (d, J=7.1 Hz, 3H). MS (ESI⁺) m/z 580 (M+Na)⁺.

### Example 717

### 2-{(4S)-5′-[1-(2-amino-2-oxoethyl)-1H-pyrazol-4-yl]-2,3′,5-trioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide

The title compound was prepared as described in EXAMPLE 295, substituting 2-[(4S)-5′-bromo-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.53 (s, 1H), 8.20 (s, 1H), 8.01-7.90 (m, 2H), 7.83 (s, 1H), 7.33 (t, J=6.7 Hz, 2H), 7.10 (t, J=8.6 Hz, 2H), 6.90 (s, 2H), 5.17 (p, J=8.1 Hz, 1H), 4.90-4.14 (m, 6H), 3.22-2.87 (m, 2H), 1.35 (d, J=7.0 Hz, 4H). MS (ESI⁺) m/z 601 (M+H)⁺.

### Example 719

### 2-[(1R)-5-(3,4-dihydro-2H-pyrrol-5-ylamino)-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

Trifluoromethanesulfonic anhydride (63.4 μl, 0.375 mmol) was slowly added dropwise to a solution of 2-pyrrolidine (25.6 mg, 0.300 mmol) and pyridine (60.7 μl, 0.751 mmol) in dichloromethane (1252 μl) at −40° C. The mixture was allowed to warm to 0° C. over 3 hours. The mixture was cooled back to −40° C. and 2-[(1R)-5-amino-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide (120 mg, 0.250 mmol) was introduced in one portion. The mixture was then allowed to slowly warm to room temperature and was stirred overnight. The solution was concentrated and purified by silica gel column chromatography to afford the title compound. ¹H NMR (400 MHz, dimethylsulfoxide-d₆) δ ppm 1.35 (dd, J=16.9, 6.9 Hz, 3H), 2.03-2.20 (m, 2H), 2.50-2.79 (m, 2H), 2.91-3.02 (m, 2H), 3.02-3.23 (m, 2H), 3.51-3.70 (m, 2H), 4.16-4.67 (m, 2H), 4.69-5.49 (m, 3H), 7.04-7.66 (m, 7H), 10.58 (s, 1H). MS (ESI⁺) m/z 547 (M+H)⁺.

### Example 724

### 2-{(3′S,4S)-5′-[1-(2-amino-2-oxoethyl)-1H-pyrazol-4-yl]-3′-hydroxy-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide

The title compound was prepared as described in EXAMPLE 570A, substituting 2-{(4S)-5′-[1-(2-amino-2-oxoethyl)-1H-pyrazol-4-yl]-2,3′,5-trioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide. ¹H NMR (400 MHz, DMSO-d₆) δ 8.34 (s, 1H), 8.04 (s, 1H), 7.80 (s, 1H), 7.56 (s, 1H), 7.48 (d, J=8.2 Hz, 1H), 7.34 (dd, J=8.5, 5.3 Hz, 2H), 7.25 (d, J=8.0 Hz, 1H), 7.11 (t, J=8.7 Hz, 2H), 6.88 (s, 2H), 5.32 (q, J=6.6 Hz, 1H), 5.17 (h, J=7.7 Hz, 1H), 4.91 (t, J=5.1 Hz, 1H), 4.86-4.21 (m, 6H), 2.66-2.51 (m, 1H), 2.39 (dd, J=13.5, 6.1 Hz, 1H), 1.35 (d, J=7.0 Hz, 3H). MS (ESI⁺) m/z 603 (M+H)⁺.

### Example 725

### 2-{5-[1-(2-amino-2-oxoethyl)-1H-pyrazol-4-yl]-6-fluoro-2′,4′-dioxo-2,3-dihydro-3′H-spiro[indene-1,5′-[1,3]oxazolidin]-3′-yl}-N-(4-fluorobenzyl)-N-[(2S)-1,1,1-trifluoropropan-2-yl]acetamide

The title compound was prepared as described in EXAMPLE 295, substituting 2-(5-bromo-6-fluoro-2′,4′-dioxo-2,3-dihydro-3′H-spiro[indene-1,5′-[1,3]oxazolidin]-3′-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.16 (s, 1H), 7.93 (d, J=22.9 Hz, 1H), 7.77 (d, J=7.1 Hz, 1H), 7.62-7.03 (m, 7H), 5.52-5.31 (m, 0.7H), 5.14-4.34 (m, 5H), 4.20 (dd, J=17.0, 9.4 Hz, 0.7H), 3.14 (dd, J=23.7, 18.5 Hz, 2H), 2.76-2.51 (m, 2H), 1.44-1.26 (m, 3H). MS (ESI⁺) m/z 606 (M+H)⁺.

### Example 726

### tert-butyl 4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]piperidine-1-carboxylate

### Example 726A

### tert-butyl 4-((4-fluorobenzyl)amino)piperidine-1-carboxylate

The title compound was prepared as described in EXAMPLE 283A, substituting tert-butyl 4-((4-fluorobenzyl)amino)piperidine-1-carboxylate for (S)-1-cyclopropylethanamine and 4-fluorobenzaldehyde for benzaldehyde.

### Example 726B

### tert-butyl 4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]piperidine-1-carboxylate

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and tert-butyl 4-((4-fluorobenzyl)amino)piperidine-1-carboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (501 MHz, DMSO-d₆) δ 8.69 (d, J=2.2 Hz, 1H), 7.55 (dd, J=8.3, 1.9 Hz, 1H), 7.38 (dt, J=9.3, 5.1 Hz, 2H), 7.27-7.20 (m, 2H), 7.17-7.06 (m, 2H), 6.08 (q, J=4.8 Hz, 1H), 4.75 (d, J=2.3 Hz, 1H), 4.68 (s, 1H), 4.56 (q, J=16.0 Hz, 1H), 4.47-4.31 (m, 2H), 3.96 (m, 2H), 3.81 (d, J=13.2 Hz, 1H), 3.12 (dt, J=16.3, 8.2 Hz, 1H), 3.00 (dtd, J=16.5, 9.4, 3.5 Hz, 1H), 2.64 (m, 4H), 2.56-2.44 (m, 1H), 1.79 (dd, J=13.2, 3.9 Hz, 1H), 1.62 (d, J=12.5 Hz, 1H), 1.51 (dt, J=9.7, 6.5 Hz, 3H), 1.38 (s, 9H). MS (ESI⁺) m/z 524 (M-99)⁺.

### Example 727

### 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(3R)-pyrrolidin-3-yl]acetamide

### Example 727A

### (R)-tert-butyl 2-(5-bromo-2′,4′-dioxo-2,3-dihydrospiro[indene-1,5′-oxazolidin]-3′-yl)acetate

The title compound was prepared as described in EXAMPLE 281F, substituting tert-butyl 2-bromoacetate for (S)-2-bromo-N-(4-fluorobenzyl)-N-(1,1,1-trifluoropropan-2-yl)acetamide.

### Example 727B

### (R)-2-(5-bromo-2′,4′-dioxo-2,3-dihydrospiro[indene-1,5′-oxazolidin]-3′-yl)acetic acid

The title compound was prepared as described in EXAMPLE 302, substituting (R)-tert-butyl 2-(5-bromo-2′,4′-dioxo-2,3-dihydrospiro[indene-1,5′-oxazolidin]-3′-yl)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 727C

### (R)-tert-butyl 3-(2-((R)-5-bromo-2′,4′-dioxo-2,3-dihydrospiro[indene-1,5′-oxazolidin]-3′-yl)-N-(4-fluorobenzyl)acetamido)pyrrolidine-1-carboxylate

The title compound was prepared as described in EXAMPLE 306, substituting (R)-2-(5-bromo-2′,4′-dioxo-2,3-dihydrospiro[indene-1,5′-oxazolidin]-3′-yl)acetic acid for cyclopropanecarboxylic acid and (R)-tert-butyl 3-((4-fluorobenzyl)amino)pyrrolidine-1-carboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 727D

### 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(3R)-pyrrolidin-3-yl]acetamide

The title compound was prepared as described in EXAMPLE 302, substituting (R)-tert-butyl 3-(2-((R)-5-bromo-2′,4′-dioxo-2,3-dihydrospiro[indene-1,5′-oxazolidin]-3′-yl)-N-(4-fluorobenzyl)acetamido)pyrrolidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 7.61 (s, 1H), 7.49 (d, J=8.1 Hz, 1H), 7.44-7.29 (m, 3H), 7.17 (t, J=8.6 Hz, 2H), 4.70 (m, 2H), 4.64-4.37 (m, 3H), 3.41 (td, J=11.7, 11.3, 7.4 Hz, 2H), 3.14 (dddd, J=36.2, 16.8, 10.1, 5.5 Hz, 4H), 2.71 (ddd, J=14.7, 8.5, 6.1 Hz, 1H), 2.52 (ddd, J=20.2, 9.6, 5.4 Hz, 1H), 2.15 (qd, J=8.1, 3.8 Hz, 1H), 2.02 (dd, J=13.3, 8.3 Hz, 1H); MS (ESI⁺) m/z 689 (M+Na)⁺.

### Example 728

### ethyl ({(3R)-3-[{[(1R)-5-bromo-2′,4′-dioxo-2,3-dihydro-3′H-spiro[indene-1,5′-[1,3]oxazolidin]-3′-yl]acetyl}(4-fluorobenzyl)amino]pyrrolidin-1-yl}sulfonyl)carbamate

To a solution of 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(3R)-pyrrolidin-3-yl]acetamide (300 mg, 0.58 mmol) in dichloromethane (20 ml) was added ((4-(dimethylamino)pyridin-1-ium-1-yl)sulfonyl)(ethoxycarbonyl)amide (175 mg, 0.64 mmol) followed by addition of triethylamine. The mixture was stirred at room temperature for 3 hours. The reaction was concentrated and the residue was purified by silica gel column chromatography to afford the title compound. ¹H NMR (501 MHz, DMSO-d₆) δ 7.67 (t, J=2.0 Hz, 1H), 7.56 (ddd, J=13.1, 8.2, 1.8 Hz, 1H), 7.43 (dd, J=25.5, 8.2 Hz, 1H), 7.33 (dd, J=8.6, 5.4 Hz, 1H), 7.29-7.23 (m, 1H), 7.20 (dd, J=8.6, 5.5 Hz, 1H), 7.15 (t, J=8.8 Hz, 1H), 4.92-4.81 (m, 1H), 4.75 (d, J=23.9 Hz, 2H), 4.59 (m, 1H), 4.48-4.27 (m, 1H), 4.19-3.95 (m, 3H), 3.63 (ddd, J=82.4, 9.7, 7.9 Hz, 1H), 3.51-3.42 (m, 1H), 3.32-3.24 (m, 1H), 3.18 (dq, J=17.0, 8.8, 7.9 Hz, 2H), 3.13-3.03 (m, 1H), 2.75-2.62 (m, 1H), 2.62-2.52 (m, 1H), 1.99-1.91 (m, 1H), 1.21-1.17 (m, 3H). MS (ESI⁺) m/z 524 (M+H)⁺.

### Example 729

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-(piperidin-4-yl)acetamide

The title compound was prepared as described in EXAMPLE 302, substituting tert-butyl 4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]piperidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (501 MHz, DMSO-d₆) δ 7.59-7.54 (m, 2H), 7.39 (dd, J=8.5, 5.3 Hz, 1H), 7.32-7.28 (m, 1H), 7.22 (dt, J=9.6, 2.0 Hz, 2H), 7.16 (q, J=8.4 Hz, 1H), δ 4.87-4.72 (m, 1H), 4.66 (m, 1H), 4.54 (q, J=16.4 Hz, 1H), 4.47-4.35 (m, 2H), 4.20 (q, J=6.8, 5.5 Hz, 2H), 3.12 (dt, J=15.5, 7.8 Hz, 1H), 3.06-2.88 (m, 2H), 2.64 (m, 4H), 2.58-2.42 (m, 1H), 2.30-2.18 (m, 1H), 1.96-1.65 (m, 4H). MS (ESI⁺) m/z 524 (M+H)⁺.

### Example 730

### ethyl ({4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]piperidin-1-yl}sulfonyl)carbamate

The title compound was prepared described as in EXAMPLE 728, substituting N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-(piperidin-4-yl)acetamide for 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(3R)-pyrrolidin-3-yl]acetamide. ¹H NMR (400 MHz, DMSO-d₆) δ 8.70 (d, J=2.3 Hz, 1H), 7.57 (d, J=1.9 Hz, 1H), 7.48 (dd, J=8.5, 5.6 Hz, 1H), 7.38 (dd, J=8.6, 5.5 Hz, 1H), 7.33-7.18 (m, 3H), 7.16-7.07 (m, 1H), 6.11 (t, J=4.9 Hz, 1H), 4.75 (m, 1H), 4.68 (m, 1H), 4.62-4.48 (m, 1H), 4.41-4.27 (m, 1H), 4.02-3.89 (m, 4H), 3.56 (tt, J=8.0, 4.7 Hz, 3H), 2.91-2.66 (m, 2H), 2.64 (m, 4H), 1.99 (dt, J=9.9, 3.8 Hz, 1H), 1.73-1.54 (m, 3H), 1.45 (tt, J=11.6, 6.0 Hz, 1H), 1.21-1.17 (m, 3H). MS (ESI⁺) m/z 673 (M−H)⁺.

### Example 731

### 2-[(3′S,4S)-5′-{1-[2-(dimethylamino)-2-oxoethyl]-1H-pyrazol-4-yl}-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 295, substituting 2-[(3′S,4S)-5′-bromo-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and boronate for 5-(4,4,5,5-tetramethyl-1,3,2-dioxaborolan-2-yl)pyridin-2-ol. ¹H NMR (400 MHz, DMSO-d₆) δ 8.35 (s, 1H), 7.99 (s, 1H), 7.77 (s, 1H), 7.55 (s, 1H), 7.52-7.06 (m, 6H), 5.32 (q, J=6.6 Hz, 1H), 5.19 (p, J=7.8 Hz, 1H), 5.04 (s, 2H), 4.91 (d, J=6.8 Hz, 1H), 4.89-4.18 (m, 4H), 2.96 (s, 6H), 2.52 (dd, J=13.4, 6.8 Hz, 1H), 2.39 (dd, J=13.5, 6.1 Hz, 1H), 1.35 (d, J=7.0 Hz, 3H). MS (ESI⁺) m/z 631 (M+H)⁺.

### Example 735

### 2-[(3′R,4S)-5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 514, substituting 2-[(3′S,4S)-5′-{1-[2-(dimethylamino)-2-oxoethyl]-1H-pyrazol-4-yl}-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide. The reaction gave a mixture of diastereomers with the title compound, 2-[(3′R,4S)-5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide, as the major disatereomer and 2-[(3′S,4S)-5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide as the minor diastereomer. ¹H NMR (400 MHz, DMSO-d₆) δ 8.60 (s, 1H), 8.05 (s, 1H), 7.83 (s, 1H), 7.75-7.56 (m, 2H), 7.32 (t, J=8.1 Hz, 3H), 7.10 (t, J=8.6 Hz, 2H), 6.12 (dt, J=57.7, 5.7 Hz, 1H), 5.17 (dq, J=15.5, 7.5 Hz, 1H), 5.04 (s, 2H), 4.86-4.12 (m, 4H), 3.04 (dt, J=13.4, 6.9 Hz, 1H), 2.96 (s, 6H), 2.39 (ddd, J=25.6, 14.3, 4.5 Hz, 1H), 1.34 (d, J=7.0 Hz, 3H). MS (ESI⁺) m/z 633 (M+H)⁺.

### Example 736

### N-(4-fluorobenzyl)-N-(3-methoxyphenyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 648B, substituting 3-methoxy-N-(4-fluorobenzyl)aniline for N-(4-fluorobenzyl)pyridin-3-amine. ¹H NMR (400 MHz, DMSO-d₆, 120° C.) δ ppm 8.27 (brs, 1H), 7.48 (s, 1H), 7.32 (t, J=8.0 Hz, 1H), 7.28-7.17 (m, 4H), 7.10-6.99 (m, 2H), 6.94 (dd, J=8.3, 2.4 Hz, 1H), 6.84-6.75 (m, 2H), 5.84 (brs, 1H), 4.89 (s, 2H), 4.23-4.07 (m, 2H), 3.74 (s, 3H), 3.20-2.99 (m, 2H), 2.70-2.57 (m, 4H), 2.46-2.38 (m, 1H). MS (ESI⁺) m/z 547 (M+H)⁺.

### Example 738

### N-(4-fluorobenzyl)-2-[(4S)-5′-{1-[2-(methylamino)-2-oxoethyl]-1H-pyrazol-4-yl}-2,5-dioxo-1H-spiro[imidazolidine-4,1′-inden]-1-yl]-N-[(2 S)-1,1,1-trifluoropropan-2-yl]acetamide

A mixture of N-(3,5-difluorobenzyl)-2-[(3′S,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide (10 mg, 0.016 mmol) and 4-methylbenzenesulfonic acid hydrate (0.617 mg, 3.24 μmol) in toluene (0.3 ml) was heated to 105° C. for 18 hours. Concentration and silica gel column chromatography provided the title compound. ¹H NMR (400 MHz, DMSO-d₆) δ 8.66 (d, J=9.8 Hz, 1H), 8.16 (d, J=4.9 Hz, 1H), 8.03 (q, J=4.5 Hz, 1H), 7.91 (d, J=5.3 Hz, 1H), 7.60 (d, J=1.4 Hz, 1H), 7.51-7.00 (m, 7H), 6.35 (dd, J=19.4, 5.5 Hz, 1H), 5.49-4.08 (m, 7H), 2.63 (dd, J=4.5, 1.5 Hz, 3H), 1.40-1.26 (m, 3H). MS (ESI(+)) m/e 599 (M+H)⁺.

### Example 739

### N-(3,4-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

### Example 739A

### (S)-2-bromo-N-(3,4-difluorobenzyl)-N-(1,1,1-trifluoropropan-2-yl)acetamide

The title compound was prepared as described in EXAMPLE 281A-B, substituting 1-(bromomethyl)-3,4-difluorobenzene for 1-(bromomethyl)-4-fluorobenzene in EXAMPLE 281A.

### Example 739B

### (S)-5′-(1-methyl-1H-pyrazol-4-yl)-2′,3′-dihydrospiro[imidazolidine-4,1′-indene]-2,5-dione

The title compound was prepared as described in EXAMPLE 295, substituting (S)-5′-bromo-2′,3′-dihydrospiro[imidazolidine-4,1′-indene]-2,5-dione for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol.

### Example 739C

### N-(3,4-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 281F, substituting (S)-5′-(1-methyl-1H-pyrazol-4-yl)-2′,3′-dihydrospiro[imidazolidine-4,1′-indene]-2,5-dione for (R)-5-bromo-2,3-dihydrospiro[indene-1,5′-oxazolidine]-2′,4′-dione and (S)-2-bromo-N-(3,4-difluorobenzyl)-N-(1,1,1-trifluoropropan-2-yl)acetamide for (S)-2-bromo-N-(4-fluorobenzyl)-N-(1,1,1,1-trifluoropropan-2-yl)acetamide. ¹H NMR (400 MHz, DMSO-d₆) δ 8.34 (s, 1H), 7.95 (s, 1H), 7.72 (s, 1H), 7.44 (s, 1H), 7.41-7.04 (m, 5H), 5.23-3.99 (m, 5H), 3.84 (s, 3H), 3.05 (t, J=7.2 Hz, 2H), 2.59 (dd, J=13.3, 6.7 Hz, 1H), 2.25 (dt, J=13.2, 7.7 Hz, 1H), 1.35 (d, J=6.8 Hz, 3H). MS (ESI(+)) m/e 562 (M+H)⁺.

### Example 740

### N-(4-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

### Example 740A

### (S)-2-bromo-N-(4-chlorobenzyl)-N-(1,1,1-trifluoropropan-2-yl)acetamide

The title compound was prepared as described in EXAMPLE 281A-B, substituting 1-(bromomethyl)-4-chlorobenzene for 1-(bromomethyl)-4-fluorobenzene in EXAMPLE 281A.

### Example 740B

### N-(4-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 281F, substituting (S)-5′-(1-methyl-1H-pyrazol-4-yl)-2′,3′-dihydrospiro[imidazolidine-4,1′-indene]-2,5-dione for (R)-5-bromo-2,3-dihydrospiro[indene-1,5′-oxazolidine]-2′,4′-dione and (S)-2-bromo-N-(4-chlorobenzyl)-N-(1,1,1-trifluoropropan-2-yl)acetamide for (S)-2-bromo-N-(4-fluorobenzyl)-N-(1,1,1,1-trifluoropropan-2-yl)acetamide. ¹H NMR (400 MHz, DMSO-d₆) δ 8.33 (d, J=3.0 Hz, 1H), 7.95 (s, 1H), 7.72 (s, 1H), 7.43 (s, 1H), 7.40-7.15 (m, 6H), 5.26-4.15 (m, 5H), 3.84 (s, 3H), 3.05 (t, J=7.2 Hz, 2H), 2.57 (dt, J=13.4, 6.7 Hz, 1H), 2.24 (dt, J=13.3, 7.7 Hz, 1H), 1.34 (d, J=7.0 Hz, 3H). MS (ESI(+)) m/e 560 (M+H)⁺.

### Example 741

### N-(3,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

### Example 741A

### (S)-2-bromo-N-(3,5-difluorobenzyl)-N-(1,1,1-trifluoropropan-2-yl)acetamide

The title compound was prepared as described in EXAMPLE 281A-B, substituting 1-(bromomethyl)-3,5-difluorobenzene for 1-(bromomethyl)-4-fluorobenzene in EXAMPLE 281A.

### Example 741B

### N-(3,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 281F, substituting (S)-5′-(1-methyl-1H-pyrazol-4-yl)-2′,3′-dihydrospiro[imidazolidine-4,1′-indene]-2,5-dione for (R)-5-bromo-2,3-dihydrospiro[indene-1,5′-oxazolidine]-2′,4′-dione and (S)-2-bromo-N-(3,5-difluorobenzyl)-N-(1,1,1-trifluoropropan-2-yl)acetamide for (S)-2-bromo-N-(4-fluorobenzyl)-N-(1,1,1,1-trifluoropropan-2-yl)acetamide. ¹H NMR (400 MHz, DMSO-d₆) δ 8.39-8.29 (m, 1H), 7.95 (s, 1H), 7.72 (s, 1H), 7.47-6.87 (m, 6H), 5.25-4.11 (m, 5H), 3.84 (s, 3H), 3.18-2.91 (m, 2H), 2.59 (dd, J=13.4, 6.7 Hz, 1H), 2.25 (dt, J=13.3, 7.7 Hz, 1H), 1.36 (d, J=7.1 Hz, 3H). MS (ESI(+)) m/e 562 (M+H)⁺.

### Example 742

### N-(3-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

### Example 742A

### (S)-2-bromo-N-(3-fluorobenzyl)-N-(1,1,1-trifluoropropan-2-yl)acetamide

The title compound was prepared as described in EXAMPLE 281A-B, substituting 1-(bromomethyl)-3-fluorobenzene for 1-(bromomethyl)-4-fluorobenzene in EXAMPLE 281A.

### Example 742B

### N-(3-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 281F, substituting (S)-5′-(1-methyl-1H-pyrazol-4-yl)-2′,3′-dihydrospiro[imidazolidine-4,1′-indene]-2,5-dione for (R)-5-bromo-2,3-dihydrospiro[indene-1,5′-oxazolidine]-2′,4′-dione and (S)-2-bromo-N-(3-fluorobenzyl)-N-(1,1,1-trifluoropropan-2-yl)acetamide for (S)-2-bromo-N-(4-fluorobenzyl)-N-(1,1,1,1-trifluoropropan-2-yl)acetamide. ¹H NMR (400 MHz, DMSO-d₆) δ 8.33 (s, 1H), 7.95 (s, 1H), 7.72 (s, 1H), 7.49-6.93 (m, 7H), 5.27-4.16 (m, 5H), 3.84 (s, 3H), 3.05 (t, J=7.2 Hz, 2H), 2.58 (dd, J=13.4, 6.7 Hz, 1H), 2.24 (dt, J=13.5, 7.7 Hz, 1H), 1.35 (d, J=7.0 Hz, 3H). MS (ESI(+)) m/e 544 (M+H)⁺.

### Example 743

### N-(2,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

### Example 743A

### (S)-2-bromo-N-(2,5-difluorobenzyl)-N-(1,1,1-trifluoropropan-2-yl)acetamide

The title compound was prepared as described in EXAMPLE 281A-B, substituting 1-(bromomethyl)-2,5-difluorobenzene for 1-(bromomethyl)-4-fluorobenzene in EXAMPLE 281A.

### Example 743B

### N-(2,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 281F, substituting (S)-5′-(1-methyl-1H-pyrazol-4-yl)-2′,3′-dihydrospiro[imidazolidine-4,1′-indene]-2,5-dione for (R)-5-bromo-2,3-dihydrospiro[indene-1,5′-oxazolidine]-2′,4′-dione and (S)-2-bromo-N-(2,5-difluorobenzyl)-N-(1,1,1-trifluoropropan-2-yl)acetamide for (S)-2-bromo-N-(4-fluorobenzyl)-N-(1,1,1,1-trifluoropropan-2-yl)acetamide. ¹H NMR (400 MHz, DMSO-d₆) δ 8.36 (s, 1H), 7.95 (s, 1H), 7.72 (s, 1H), 7.48-6.97 (m, 6H), 5.23-4.27 (m, 5H), 3.84 (s, 3H), 3.05 (t, J=7.2 Hz, 2H), 2.58 (dt, J=13.3, 6.7 Hz, 1H), 2.25 (dt, J=13.5, 7.7 Hz, 1H), 1.37 (d, J=6.9 Hz, 3H). MS (ESI(+)) m/e 562 (M+H)⁺.

### Example 744

### N-(3-chlorobenzyl)-2-[(4 S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

### Example 744A

### (S)-2-bromo-N-(3-chlorobenzyl)-N-(1,1,1-trifluoropropan-2-yl)acetamide

The title compound was prepared as described in EXAMPLE 281A-B, substituting 1-(bromomethyl)-3-chlorobenzene for 1-(bromomethyl)-4-fluorobenzene in EXAMPLE 281A.

### Example 744B

### N-(3-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 281F, substituting (S)-5′-(1-methyl-1H-pyrazol-4-yl)-2′,3′-dihydrospiro[imidazolidine-4,1′-indene]-2,5-dione for (R)-5-bromo-2,3-dihydrospiro[indene-1,5′-oxazolidine]-2′,4′-dione and ((S)-2-bromo-N-(3-chlorobenzyl)-N-(1,1,1-trifluoropropan-2-yl)acetamide for (S)-2-bromo-N-(4-fluorobenzyl)-N-(1,1,1,1-trifluoropropan-2-yl)acetamide. ¹H NMR (400 MHz, DMSO-d₆) δ 8.36 (s, 1H), 7.97 (s, 1H), 7.74 (s, 1H), 7.49-7.16 (m, 7H), 5.27-4.14 (m, 5H), 3.86 (s, 3H), 3.07 (t, J=7.2 Hz, 2H), 2.59 (dt, J=13.3, 6.7 Hz, 1H), 2.26 (dt, J=13.4, 7.7 Hz, 1H), 1.36 (d, J=6.9 Hz, 3H). MS (ESI(+)) m/e 560 (M+H)⁺.

### Example 745

### N-[(1S)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide

The title compound was prepared as described in EXAMPLE 281F, substituting (S)-5′-(1-methyl-1H-pyrazol-4-yl)-2′,3′-dihydrospiro[imidazolidine-4,1′-indene]-2,5-dione for (R)-5-bromo-2,3-dihydrospiro[indene-1,5′-oxazolidine]-2′,4′-dione and (S)-2-bromo-N-(1-cyclopropylethyl)-N-(4-fluorobenzyl)acetamide for (S)-2-bromo-N-(4-fluorobenzyl)-N-(1,1,1-trifluoropropan-2-yl)acetamide. ¹H NMR (400 MHz, DMSO-d₆) δ 8.28 (d, J=3.6 Hz, 1H), 7.95 (s, 1H), 7.72 (s, 1H), 7.49-7.01 (m, 7H), 4.64 (s, 2H), 4.25 (d, J=3.3 Hz, 2H), 3.84 (s, 3H), 3.65-3.50 (m, 1H), 3.15-2.87 (m, 2H), 2.58 (dt, J=13.3, 6.7 Hz, 1H), 2.24 (dt, J=13.2, 7.7 Hz, 1H), 1.16 (d, J=6.7 Hz, 3H), 1.02-0.88 (m, 1H), 0.55-0.41 (m, 1H), 0.36-0.11 (m, 3H). MS (ESI(+)) m/e 516 (M+H)⁺.

### Example 746

### N-[(1 S)-1-cyclopropyl-2,2,2-trifluoroethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide

### Example 746A

### (S)-2-bromo-N-(1-cyclopropyl-2,2,2-trifluoroethyl)-N-(4-fluorobenzyl)acetamide

The title compound was prepared as described in EXAMPLE 281A-B, substituting (S)-1-cyclopropyl-2,2,2-trifluoroethanamine hydrochloride for (S)-1,1,1-trifluoropropan-2-amine hydrochloride in EXAMPLE 281A.

### Example 746B

### N-[(1S)-1-cyclopropyl-2,2,2-trifluoroethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide

The title compound was prepared as described in EXAMPLE 281F, substituting (S)-5′-(1-methyl-1H-pyrazol-4-yl)-2′,3′-dihydrospiro[imidazolidine-4,1′-indene]-2,5-dione for (R)-5-bromo-2,3-dihydrospiro[indene-1,5′-oxazolidine]-2′,4′-dione and (S)-2-bromo-N-(1-cyclopropyl-2,2,2-trifluoroethyl)-N-(4-fluorobenzyl)acetamide for (S)-2-bromo-N-(4-fluorobenzyl)-N-(1,1,1,1-trifluoropropan-2-yl)acetamide. ¹H NMR (400 MHz, DMSO-d₆) δ 8.37-8.27 (m, 1H), 7.95 (s, 1H), 7.72 (s, 1H), 7.50-7.01 (m, 7H), 4.96-4.11 (m, 5H), 3.84 (s, 3H), 3.05 (t, J=7.3 Hz, 2H), 2.57 (dt, J=13.3, 6.7 Hz, 1H), 2.24 (dt, J=13.5, 7.7 Hz, 1H), 1.29-1.13 (m, 1H), 0.82-0.62 (m, 2H), 0.45-0.21 (m, 2H). MS (ESI(+)) m/e 570 (M+H)⁺.

### Example 747

### N-[(1R)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide

The title compound was prepared as described in EXAMPLE 281F, substituting (S)-5′-(1-methyl-1H-pyrazol-4-yl)-2′,3′-dihydrospiro[imidazolidine-4,1′-indene]-2,5-dione for (R)-5-bromo-2,3-dihydrospiro[indene-1,5′-oxazolidine]-2′,4′-dione and (R)-2-bromo-N-(1-cyclopropylethyl)-N-(4-fluorobenzyl)acetamide for (S)-2-bromo-N-(4-fluorobenzyl)-N-(1,1,1-trifluoropropan-2-yl)acetamide. ¹H NMR (400 MHz, DMSO-d₆) δ 8.28 (s, 1H), 7.95 (s, 1H), 7.73 (s, 1H), 7.49-6.98 (m, 7H), 4.64 (s, 2H), 4.37-4.11 (m, 2H), 3.85 (s, 3H), 3.67-3.50 (m, 1H), 3.04 (d, J=6.6 Hz, 2H), 2.58 (dt, J=13.3, 6.7 Hz, 1H), 2.24 (dt, J=13.3, 7.7 Hz, 1H), 1.16 (d, J=6.7 Hz, 3H), 1.03-0.86 (m, 1H), 0.58-0.40 (m, 1H), 0.35-0.14 (m, 3H). MS (ESI(+)) m/e 516 (M+H)⁺.

### Example 748

### N-benzyl-N-[(1S)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide

The title compound was prepared as described in EXAMPLE 281F, substituting (S)-5′-(1-methyl-1H-pyrazol-4-yl)-2′,3′-dihydrospiro[imidazolidine-4,1′-indene]-2,5-dione for (R)-5-bromo-2,3-dihydrospiro[indene-1,5′-oxazolidine]-2′,4′-dione and (S)—N-benzyl-2-bromo-N-(1-cyclopropylethyl)acetamide for (S)-2-bromo-N-(4-fluorobenzyl)-N-(1,1,1-trifluoropropan-2-yl)acetamide. ¹H NMR (400 MHz, DMSO-d₆) δ 8.27 (d, J=3.8 Hz, 1H), 7.95 (s, 1H), 7.72 (s, 1H), 7.48-7.15 (m, 8H), 4.66 (s, 2H), 4.33-4.12 (m, 2H), 3.84 (s, 3H), 3.65-3.55 (m, 1H), 3.05 (t, J=7.2 Hz, 2H), 2.58 (dt, J=13.4, 6.7 Hz, 1H), 2.24 (dt, J=13.2, 7.7 Hz, 1H), 1.16 (d, J=6.7 Hz, 3H), 1.02-0.90 (m, 1H), 0.54-0.43 (m, 1H), 0.37-0.17 (m, 3H). MS (ESI(+)) m/e 498 (M+H)⁺.

### Example 749

### N-benzyl-N-[(1R)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide

### Example 749A

### (R)—N-benzyl-2-bromo-N-(1-cyclopropylethyl)acetamide

The title compound was prepared as described in EXAMPLE 283A-B, substituting (R)-1-cyclopropylethanamine for (S)-1-cyclopropylethanamine in EXAMPLE 283A.

### Example 749B

### N-benzyl-N-[(1R)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide

The title compound was prepared as described in EXAMPLE 281F, substituting (S)-5′-(1-methyl-1H-pyrazol-4-yl)-2′,3′-dihydrospiro[imidazolidine-4,1′-indene]-2,5-dione for (R)-5-bromo-2,3-dihydrospiro[indene-1,5′-oxazolidine]-2′,4′-dione and (R)—N-benzyl-2-bromo-N-(1-cyclopropylethyl)acetamide for (S)-2-bromo-N-(4-fluorobenzyl)-N-(1,1,1-trifluoropropan-2-yl)acetamide. ¹H NMR (400 MHz, DMSO-d₆) δ 8.26 (d, J=3.3 Hz, 1H), 7.95 (s, 1H), 7.72 (s, 1H), 7.50-7.13 (m, 8H), 4.66 (s, 2H), 4.33-4.12 (m, 2H), 3.84 (s, 3H), 3.67-3.55 (m, 1H), 3.04 (d, J=6.9 Hz, 2H), 2.59 (dd, J=13.3, 6.7 Hz, 1H), 2.24 (dt, J=14.2, 7.7 Hz, 1H), 1.15 (d, J=6.6 Hz, 3H), 1.03-0.90 (m, 1H), 0.54-0.43 (m, 1H), 0.34-0.18 (m, 3H). MS (ESI(+)) m/e 498 (M+H)⁺.

### Example 750

### 2-(4′-bromo-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

### Example 750A

### 4′-bromo-2′,3′-dihydrospiro[imidazolidine-4,1′-indene]-2,5-dione

The title compound was prepared as described in EXAMPLE 290B, substituting 4-bromo-2,3-dihydro-1H-inden-1-one for 6-bromobenzo[b]thiophen-3 (2H)-one 1,1-dioxide.

### Example 750B

### 2-(4′-bromo-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

The title compound was prepared as described in EXAMPLE 281F, substituting 4′-bromo-2′,3′-dihydrospiro[imidazolidine-4,1′-indene]-2,5-dione for (R)-5-bromo-2,3-dihydrospiro[indene-1,5′-oxazolidine]-2′,4′-dione. ¹H NMR (400 MHz, DMSO-d₆) δ 9.01-8.78 (m, 1H), 7.51 (dd, J=17.1, 8.2 Hz, 1H), 7.41-6.96 (m, 6H), 5.32 (dt, J=15.9, 8.0 Hz, 0.7H), 5.03 (d, J=5.5 Hz, 0.3H), 4.93-4.58 (m, 2H), 4.49-4.25 (m, 1.3H), 4.12-3.92 (m, 0.7H), 3.09-2.84 (m, 2H), 2.66-2.49 (m, 1H), 2.31-2.08 (m, 1H), 1.29 (dd, J=25.9, 6.8 Hz, 3H). MS (ESI⁻) m/z 542 (M−H)⁻.

### Example 751

### N-(4-fluorobenzyl)-2-[4′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 295, substituting 2-(4′-bromo-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.89-8.75 (m, 1H), 8.08 (s, 1H), 7.80 (d, J=12.9 Hz, 1H), 7.57-7.46 (m, 1H), 7.42-7.32 (m, 1H), 7.32-7.02 (m, 4H), 5.45-5.24 (m, 1H), 5.05 (s, 1H), 4.96-4.61 (m, 2H), 4.45-4.29 (m, 1H), 4.14-4.00 (m, 1H), 3.89 (s, 3H), 3.14 (dd, J=10.5, 5.3 Hz, 2H), 2.63-2.49 (m, 1H), 2.24 (dd, J=17.5, 9.9 Hz, 1H), 1.31 (m, 3H). MS (ESI⁺) m/z 544 (M+H)⁺.

### Example 752

### N-(4-fluorobenzyl)-2-(6-fluoro-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl)-N-[(2S)-1, 1,1-trifluoropropan-2-yl]acetamide

The title compound was prepared as described in EXAMPLE 295, substituting 2-(5-amino-6-fluoro-2′,4′-dioxo-2,3-dihydro-3′H-spiro[indene-1,5′-[1,3]oxazolidin]-3′-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.14 (t, J=11.7 Hz, 1H), 8.09-7.90 (m, 2H), 7.74 (t, J=11.9 Hz, 1H), 7.64-7.01 (m, 5H), 5.37 (dd, J=24.9, 17.4 Hz, 0.7H), 5.12-4.35 (m, 5.6H), 4.18 (dd, J=17.0, 9.1 Hz, 0.7H), 3.21-2.92 (m, 2H), 2.78-2.50 (m, 5H), 1.43-1.24 (m, 3H). MS (ESI⁺) m/z 620.2 (M+H)⁺.

### Example 753

### 2-[(3′S,4S)-5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 514, substituting 2-[(3′S,4S)-5′-{1-[2-(dimethylamino)-2-oxoethyl]-1H-pyrazol-4-yl}-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide. The reaction gave a mixture of diastereomers with the title compound, 2-[(3′S,4S)-5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide, as the minor diastereomer and 2-[(3′R,4S)-5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide as the major diastereomer. ¹H NMR (400 MHz, DMSO-d₆) δ 8.32 (s, 1H), 8.05 (s, 1H), 7.83 (s, 1H), 7.75-7.56 (m, 2H), 7.34 (t, J=7.6 Hz, 3H), 7.11 (t, J=8.6 Hz, 2H), 6.28 (t, J=5.1 Hz, 1H), 5.18 (p, J=7.9, 7.4 Hz, 1H), 5.04 (s, 2H), 4.86-4.22 (m, 4H), 2.96 (s, 6H), 2.77-2.60 (m, 2H), 1.35 (d, J=6.9 Hz, 3H). MS (ESI⁺) m/z 633 (M+H)⁺.

### Example 759

### 2-[(3S)-6-amino-1,1-dioxido-2′,5′-dioxo-1′H-spiro[1-benzothiophene-3,4′-imidazolidin]-1′-yl]-N-(4-fluorobenzyl)-N-[(2S)-1,1,1-trifluoropropan-2-yl]acetamide

The diastereomers of 2-(6-amino-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide were separated by chiral SFC (Chiralcel OD-H column) to afford 2-[(3R)-6-amino-1,1-dioxido-2′,5′-dioxo-1′H-spiro[1-benzothiophene-3,4′-imidazolidin]-1′-yl]-N-(4-fluorobenzyl)-N-[(2S)-1,1,1-trifluoropropan-2-yl]acetamide (eluted first) and 2-[(3S)-6-amino-1,1-dioxido-2′,5′-dioxo-1′H-spiro[1-benzothiophene-3,4′-imidazolidin]-1′-yl]-N-(4-fluorobenzyl)-N-[(2S)-1,1,1-trifluoropropan-2-yl]acetamide (eluted second). The assignments of absolute configuration at the spiro center were made based on relative potencies of the two diastereomers and the known SAR of the hydantoin series. ¹H NMR (400 MHz, DMSO-d₆, 120° C.) δ ppm 8.67 (brs, 1H), 7.32 (dd, J=8.5, 5.3 Hz, 2H), 7.23 (d, J=8.5 Hz, 1H), 7.17-7.04 (m, 2H), 6.93 (dd, J=8.5, 2.2 Hz, 1H), 6.82 (d, J=2.2 Hz, 1H), 5.59 (brs, 2H), 5.26-5.04 (m, 1H), 4.80 (d, J=17.5 Hz, 1H), 4.60 (d, J=17.6 Hz, 1H), 4.47 (d, J=16.7 Hz, 1H), 4.26 (d, J=16.7 Hz, 1H), 3.81 (d, J=14.2 Hz, 1H), 3.64 (d, J=14.2 Hz, 1H), 1.34 (d, J=7.1 Hz, 3H). MS (ESI⁺) m/z 529 (M+H)⁺.

### Example 760

### 2-[(3′R,4S)-5′-bromo-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

### Example 760A

### 2-((1′S,3′S)-5′-bromo-3′-hydroxy-2,5-dioxo-2′,3′-dihydrospiro[imidazolidinee-4,1′-inden]-1-yl)-N-(4-fluorobenzyl)-N—((S)-1,1,1-trifluoropropan-2-yl)acetamide

The title compound was prepared as described in EXAMPLE 570A, substituting 2-[(4S)-5′-bromo-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide.

### Example 760B

### 2-[(3′R,4S)-5′-bromo-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 514, substituting 2-((1′S,3′S)-5′-bromo-3′-hydroxy-2,5-dioxo-2′,3′-dihydro spiro[imidazolidine-4,1′-inden]-1-yl)-N-(4-fluorobenzyl)-N—((S)-1,1,1-trifluoropropan-2-yl)acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide. ¹H NMR (400 MHz, DMSO-d₆) δ 8.66 (s, 1H), 7.72 (s, 1H), 7.70-7.55 (m, 1H), 7.31 (dd, J=8.4, 5.0 Hz, 3H), 7.10 (t, J=8.7 Hz, 2H), 6.11 (ddd, J=56.9, 6.7, 4.7 Hz, 1H), 5.26-5.00 (m, 1H), 4.85-4.16 (m, 4H), 3.05 (ddd, J=14.6, 12.4, 6.9 Hz, 1H), 2.39 (ddd, J=25.7, 14.4, 4.6 Hz, 1H), 1.33 (d, J=7.0 Hz, 3H). MS (ESI⁺) m/z 582 (M+Na)⁺.

### Example 761

### 2-(5′-bromo-6′-fluoro-2,3′,5-trioxo-2′,3′-dihydro-H-spiro[imidazolidine-4,1′-inden]-1-yl)-N-(4-fluorobenzyl)-N-[(2S)-1,1,1-trifluoropropan-2-yl]acetamide

### Example 761A

### 5′-bromo-6′-fluoro-2′,3′-dihydrospiro[imidazolidine-4,1′-indene]-2,5-dione

To a solution of 5-bromo-6-fluoro-2,3-dihydro-1H-inden-1-one (1.5 g, 6.55 mmol) in ethanol (10 ml) and water (10.00 ml) was added KCN (0.853 g, 13.10 mmol) and ammonium bicarbonate (2.071 g, 26.2 mmol) in a 100 mL flask and stirred for 16 hours at 100° C. The mixture was diluted with water and extracted with ethyl acetate. The organic layer was washed with brine, dried over Na₂SO₄, filtered and concentrated to afford the title compound.

### Example 761B

### 5′-bromo-6′-fluorospiro[imidazolidine-4,1′-indene]-2,3′,5 (2′H)-trione

To solution of compound 5′-bromo-6′-fluoro-2′,3′-dihydrospiro[imidazolidine-4,1′-indene]-2,5-dione (7 g, 18.69 mmol) in acetone (140 ml) at −78° C. was added KMnO₄ (17.78 g, 112.35 mmol) and iron(III) chloride (9.114 g, 56.14 mmol). The mixture was stirred at −78° C. for 1 hour and was allowed to slowly warm up to room temperature for 16 hours. The mixture was filtered and the solid was rinsed thoroughly with acetone. The filtrate was concentrated and purified by silica gel column chromatography to give the title compound.

### Example 761C

### 2-(5′-bromo-6′-fluoro-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

The title compound was prepared as described in EXAMPLE 281F, substituting 5′-bromo-6′-fluorospiro[imidazolidine-4,1′-indene]-2,3′,5 (2′H)-trione for (R)-5-bromo-2,3-dihydrospiro[indene-1,5′-oxazolidine]-2′,4′-dione. ¹H NMR (400 MHz, DMSO-d₆) δ 8.96 (d, J=7.2 Hz, 1H), 8.12 (t, J=6.5 Hz, 1H), 7.65 (dd, J=16.9, 8.3 Hz, 1H), 7.33 (ddd, J=8.1, 5.2, 2.6 Hz, 1.3H), 7.19 (td, J=8.8, 1.8 Hz, 2H), 7.08 (dd, J=18.3, 9.1 Hz, 0.7H), 5.34 (dt, J=15.6, 7.7 Hz, 0.7H), 5.03 (dd, J=14.0, 7.0 Hz, 0.3H), 4.94-4.60 (m, 2H), 4.53-4.30 (m, 1.3H), 4.08 (dd, J=21.5, 17.0 Hz, 0.7H), 3.20-2.90 (m, 2H), 1.41-1.19 (m, 3H). MS (ESI⁻) m/z 572 (M−H)⁻.

### Example 762

### 2-(5′-bromo-6′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

The title compound was prepared as described in EXAMPLE 281F, substituting 5′-bromo-6′-fluoro-2′,3′-dihydrospiro[imidazolidine-4,1′-indene]-2,5-dione for (R)-5-bromo-2,3-dihydrospiro[indene-1,5′-oxazolidine]-2′,4′-dione. ¹H NMR (400 MHz, DMSO-d₆) δ 8.89 (d, J=11.1 Hz, 1H), 7.69 (d, J=6.4 Hz, 1H), 7.45-7.30 (m, 1H), 7.22 (dt, J=12.8, 6.0 Hz, 2.3H), 7.09 (dd, J=16.3, 8.6 Hz, 0.7H), 5.46-5.21 (m, 0.7H), 5.05 (s, 0.3H), 4.95-4.60 (m, 2H), 4.46-4.27 (m, 1.3H), 4.14-3.96 (m, 0.7H), 3.00 (s, 2H), 2.55 (dt, J=13.5, 6.6 Hz, 1H), 2.24 (tt, J=8.0, 6.4 Hz, 1H), 1.41-1.16 (m, 3H). MS (ESI⁻) m/z 558 (M−H)⁻.

### Example 763

### N-(4-fluorobenzyl)-2-(6′-fluoro-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide

The title compound was prepared as described in EXAMPLE 295, substituting 2-(5′-bromo-6′-fluoro-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.96 (d, J=8.2 Hz, 1H), 8.27 (d, J=24.8 Hz, 1H), 8.09 (m, 2H), 7.64-7.51 (m, 1H), 7.36 (s, 1H), 7.15 (ddd, J=28.3, 14.0, 7.7 Hz, 2H), 5.47-5.27 (m, 1H), 5.07 (s, 0.6H), 4.97-4.64 (m, 3H), 4.55-4.33 (m, 1.4H), 4.25-3.99 (m, 1H), 3.22-3.03 (m, 1H), 3.03-2.88 (m, 1H), 2.61 (d, J=4.4 Hz, 3H), 1.42-1.23 (m, 3H). MS (ESI⁺) m/z 633 (M+H)⁺.

### Example 764

### N-(4-fluorobenzyl)-2-(6′-fluoro-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide

The title compound was prepared as described in EXAMPLE 570A, substituting N-(4-fluorobenzyl)-2-(6′-fluoro-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide. ¹H NMR (400 MHz, DMSO-d₆) δ 8.81 (s, 1H), 8.15 (s, 1H), 8.02 (d, J=3.8 Hz, 1H), 7.91 (s, 1H), 7.67 (d, J=7.1 Hz, 1H), 7.44-7.29 (m, 1H), 7.29-7.02 (m, 3H), 5.58 (m, 1H), 5.39 (m, 0.6H), 5.27 (m, 1H), 5.07 (m, 0.4H), 4.97-4.61 (m, 3H), 4.53-4.30 (m, 1.4H), 4.11 (dd, J=16.9, 12.4 Hz, 0.6H), 2.66-2.47 (m, 4H), 2.33 (td, J=13.4, 8.4 Hz, 1H), 1.40-1.20 (m, 3H). MS (ESI⁺) m/z 635 (M+H)⁺.

### Example 765

### N-(4-fluorobenzyl)-2-[4′-(1-methyl-1,2,3,6-tetrahydropyridin-4-yl)-2,5-dioxo-2′,3′-dihydro-1H-spiro[imidazolidine-4,1′-inden]-1-yl]-N-[(2 S)-1,1,1-trifluoropropan-2-yl]acetamide

The title compound was prepared as described in EXAMPLE 295, substituting 2-(4′-bromo-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1,2,3,6-tetrahydropyridine for 5-(4,4,5,5-tetramethyl-1,3,2-dioxaborolan-2-yl)pyridin-2-ol. ¹H NMR (400 MHz, DMSO-d₆) δ 8.89-8.64 (m, 1H), 7.33 (dt, J=21.2, 9.2 Hz, 2H), 7.26-7.01 (m, 5H), 5.77 (d, J=18.3 Hz, 1H), 5.44-5.19 (m, 0.7H), 5.11-4.60 (m, 2.3H), 4.45-4.28 (m, 1.3H), 4.08 (dd, J=23.5, 13.8 Hz, 0.7H), 3.07-2.90 (m, 4H), 2.62-2.01 (m, 11H), 1.27 (dt, J=24.3, 12.3 Hz, 3H). MS (ESI⁺) m/z 559 (M+H)⁺.

### Example 766

### 2-(3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

The title compound was prepared as described in EXAMPLE 514, substituting N-(4-fluorobenzyl)-2-(6′-fluoro-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2 S)-1,1,1,1-trifluoropropan-2-yl]acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide. ¹H NMR (400 MHz, DMSO-d₆) δ 9.09 (d, J=11.7 Hz, 1H), 8.19 (s, 1H), 7.97 (dd, J=26.5, 7.7 Hz, 2H), 7.39-7.03 (m, 5H), 6.11 (dd, J=57.7, 3.5 Hz, 1H), 5.42-5.24 (m, 0.7H), 5.03 (d, J=7.2 Hz, 0.3H), 4.93-4.61 (m, 4H), 4.47-4.30 (m, 1.3H), 4.06 (dd, J=16.8, 14.2 Hz, 0.7H), 3.05 (ddd, J=18.7, 9.9, 5.0 Hz, 1H), 2.60 (d, J=4.5 Hz, 3H), 2.34 (ddd, J=25.5, 11.2, 7.3 Hz, 1H), 1.36-1.22 (m, 3H). MS (ESI⁺) m/z 637.1 (M+H)⁺.

### Example 767

### N-[(1S)-1-cyclopropyl-2,2,2-trifluoroethyl]-N-(4-fluorobenzyl)-2-[7-(1-methyl-1H-pyrazol-4-yl)-2′,4′-dioxo-1H,3′H-spiro[isochromene-4,5′-[1,3]oxazolidin]-3′-yl]acetamide

### Example 767A

### 4-bromo-2-(bromomethyl)benzonitrile

4-Bromo-2-methylbenzonitrile 1 (80 g, 408.16 mmol), N-bromosuccinimide (72.24 g, 408.16 mmol), benzyl peroxide (197.74 g, 816.32 mmol) and carbon tetrachloride were loaded in a flask and set to reflux for 6 hours. Once cooled, the succinimide precipitate was filtered off and carbon tetrachloride was removed via a dry ice-acetone cooled rotary evaporator. The residue was dissolved in dichloromethane (300 mL), washed with distilled water (200 mL×3) and brine (200 mL×2), dried over sodium sulfate, filtered and concentrated to leave a residue. The residue was purified by silica gel column chromatography to give the title compound.

### Example 767B

### ethyl 2-((5-bromo-2-cyanobenzyl)oxy)acetate

A solution of 4-bromo-2-(bromomethyl)benzonitrile (35 g, 127.32 mmol) in dimethylsulfoxide (300 mL) was added slowly to a solution of ethyl 2-hydroxyacetate (13.25 g, 127.32 mmol) and sodium methoxide (freshly prepared with 4.39 g Na in methanol then concentrated) in DMSO (50 mL). The mixture was maintained at room temperature for 1 hour, then heated at 65° C. for 5 hours and allowed to cool to room temperature again. LCMS showed the reaction was complete. The mixture was diluted with ice water and extracted with ether (150 mL×3) and the combined organic layers were dried with sodium sulfate, filtered, and concentrated. The residue was purified by column chromatography (on silica gel, eluted with ethyl acetate:hexanes, 1:10) to provide the title compound.

### Example 767C

### 4-bromo-2-((carboxymethoxy)methyl)benzoic acid

KOH (18.79 g, 335.45 mmol) was added to a solution of ethyl 2-((5-bromo-2-cyanobenzyl)oxy)acetate (20 g, 67.09 mmol) in methanol (100 mL) and water (100 mL). The mixture was stirred at 90° C. overnight, cooled to room temperature and adjusted to the pH 1 with 1N aqueous HCl. The resulting solution was extracted with dichloromethane (150 mL×3) and the combined organic layers were dried with sodium sulfate, filtered, and concentrated to provide the title compound.

### Example 767D

### 7-bromoisochroman-4-one

4-Bromo-2-((carboxymethoxy)methyl)benzoic acid (14.5 g, 50.2 mmol) and dry potassium acetate (22.15 mg, 226 mmol) were mixed and diluted with anhydrous acetic anhydride (78 mL). The mixture was heated at 138° C. for 2 hours. The reaction mixture was cooled to room temperature and concentrated under reduced pressure and the residue was partitioned between ethyl acetate and water. The phases were separated and the aqueous phase was extracted with ethyl acetate. The combined organic phase was then washed with saturated sodium chloride, dried over sodium sulfate, filtered and concentrated. The residue was purified by silica gel column chromatography to obtain the acetate intermediate (9.3 g). The acetate intermediate (9.3 g, 34.4 mmol) was added to a solution of lithium hydroxide (1.65 g, 68.9 mmol) and methanol/water (30 mL/30 mL). The mixture was stirred at room temperature for 2 hours. The solution was extracted with ethyl acetate and then washed with saturated sodium chloride, dried over sodium sulfate, filtered and concentrated to provide the title compound.

### Example 767E

### 7-bromo-4-((trimethylsilyl)oxy)isochroman-4-carbonitrile

To a stirring solution of 7-bromoisochroman-4-one (7.6 g, 33.48 mmol) in dichloromethane (80 mL) was added 4-methylmorpholine 4-oxide (2.35 g, 20.09 mmol) and trimethylsilylcyanide (9.96 g, 100.44 mmol). The mixture was stirred at room temperature for 16 hours. The residue was slurried in 100 mL of hexanes for 15 minutes and filtered through a pad of diatomaceous earth to remove solids. The filtrate was concentrated to give the title compound which was used without further purification.

### Example 767F

### ethyl 7-bromo-4-hydroxyisochroman-4-carboxylate

A three neck 1 L flask was fitted with a thermocouple, HCl gas in (diffussor) and HCl out, vented out the back of the hood. 7-Bromo-4-((trimethylsilyl)oxy)isochroman-4-carbonitrile (8.8 g, 26.99 mmol) was taken up into ethanol (100 ml) and cooled to 0° C. HCl (g) (36.26 g, 993.4 mmol) was gassed in carefully over 4 hours under nitrogen. The temperature was kept below 25° C. with an ice bath. The mixture was stirred for an additional hour, and the solvent was evaporated in vacuo. The product was purified by silica gel column chromatography to afford the title compound.

### Example 767G

### 7-bromospiro[isochroman-4,5′-oxazolidine]-2′,4′-dione

To a stirring solution of compound ethyl 7-bromo-4-hydroxyisochroman-4-carboxylate (5.8 g, 19.26 mmol) in dichloromethane (50 ml) at 0° C. was added 2,2,2-trichloroacetyl isocyanate (7.26 g, 38.5 mmol) dropwise. After the addition, the resulting mixture was allowed to stir at room temperature for 30 minutes. The excess 2,2,2-trichloroacetyl isocyanate was quenched using methanol (20 mL). The reaction mixture was concentrated in vacuo. The residue was dissolved in ethanol (50.0 ml), followed by the addition of triethylamine (0.416 ml, 2.98 mmol). The reaction mixture was then heated to reflux overnight. The solvent was removed under vacuum. The residue was suspended in hexanes/dichloroomethane (1 mL: 50 mL), stirred for 2 hours, and filtered to afford the title compound.

### Example 767H

### 2-(7-bromo-2′,4′-dioxospiro[isochroman-4,5′-oxazolidin]-3′-yl)-N—((S)-1-cyclopropyl-2,2,2-trifluoroethyl)-N-(4-fluorobenzyl)acetamide

The title compound was prepared as described in EXAMPLE 281F, substituting 7-bromospiro[isochroman-4,5′-oxazolidine]-2′,4′-dione for (R)-5-bromo-2,3-dihydrospiro[indene-1,5′-oxazolidine]-2′,4′-dione and (S)-2-bromo-N-(1-cyclopropyl-2,2,2-trifluoroethyl)-N-(4-fluorobenzyl)acetamide for (S)-2-bromo-N-(4-fluorobenzyl)-N-(1,1,1-trifluoropropan-2-yl)acetamide.

### Example 7671

### N-[(1S)-1-cyclopropyl-2,2,2-trifluoroethyl]-N-(4-fluorobenzyl)-2-[7-(1-methyl-1H-pyrazol-4-yl)-2′,4′-dioxo-1H,3′H-spiro[isochromene-4,5′-[1,3]oxazolidin]-3′-yl]acetamide

The title compound was prepared as described in EXAMPLE 295, substituting 2-(7-bromo-2′,4′-dioxospiro[isochroman-4,5′-oxazolidin]-3′-yl)-N—((S)-1-cyclopropyl-2,2,2-trifluoroethyl)-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, dimethylsulfoxide-d₆) δ ppm 0.11-0.48 (m, 2H), 0.66-0.89 (m, 2H), 1.21-1.30 (m, 1H), 3.77-3.92 (m, 3H), 3.94-5.16 (m, 9H), 7.03-7.61 (m, 7H), 7.84-7.95 (m, 1H), 8.13-8.21 (m, 1H). MS (ESI⁺) m/z 547 (M+H)⁺.

### Example 768

### 2-(7-amino-2′,4′-dioxo-1H,3′H-spiro[isochromene-4,5′-[1,3]oxazolidin]-3′-yl)-N-[(1S)-1-cyclopropyl-2,2,2-trifluoroethyl]-N-(4-fluorobenzyl)acetamide

The title compound was prepared as described in EXAMPLE 284D-E, substituting 2-(7-bromo-2′,4′-dioxospiro[isochroman-4,5′-oxazolidin]-3′-yl)-N—((S)-1-cyclopropyl-2,2,2-trifluoroethyl)-N-(4-fluorobenzyl)acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide. ¹H NMR (400 MHz, dimethylsulfoxide-d₆) δ ppm 1.31 (dd, J=25.6, 6.5 Hz, 3H), 2.13-2.32 (m, 1H), 2.50-2.64 (m, 1H), 2.91-3.13 (m, 2H), 3.96-4.48 (m, 2H), 4.63-5.47 (m, 3H), 7.03-7.64 (m, 6H), 8.70-8.92 (m, 1H). MS (ESI⁺) m/z 544 (M+H)⁺.

### Example 769

### N-[(1S)-1-cyclopropyl-2,2,2-trifluoroethyl]-N-(4-fluorobenzyl)-2-{7-[(methylcarbamoyl)amino]-2′,4′-dioxo-1H,3′H-spiro[isochromene-4,5′-[1,3]oxazolidin]-3′-yl}acetamide

The title compound was prepared as described in EXAMPLE 282, substituting 2-(7-amino-2′,4′-dioxo-1H,3′H-spiro[isochromene-4,5′-[1,3]oxazolidin]-3′-yl)-N-[(1S)-1-cyclopropyl-2,2,2-trifluoroethyl]-N-(4-fluorobenzyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, dimethylsulfoxide-d₆) δ ppm 0.13-0.44 (m, 2H), 0.66-0.85 (m, 2H), 1.19-1.30 (m, 1H), 2.60 (dd, J=4.6, 1.9 Hz, 3H), 3.93-5.12 (m, 9H), 5.99-6.22 (m, 1H), 6.97-7.53 (m, 7H), 8.69 (d, J=1.9 Hz, 1H); MS (ESI⁺) m/z 579 (M+H)⁺.

### Example 770

### N-(4-fluorobenzyl)-2-{7-[(methylcarbamoyl)amino]-2′,4′-dioxo-1H,3′H-spiro[isochromene-4,5′-[1,3]oxazolidin]-3′-yl}-N-[(2S)-1,1,1-trifluoropropan-2-yl]acetamide

### Example 770A

### 2-(7-bromo-2′,4′-dioxospiro[isochroman-4,5′-oxazolidin]-3′-yl)-N-(4-fluorobenzyl)-N—((S)-1,1,1-trifluoropropan-2-yl)acetamide

The title compound was prepared as described in EXAMPLE 281F, substituting 7-bromospiro[isochroman-4,5′-oxazolidine]-2′,4′-dione for (R)-5-bromo-2,3-dihydrospiro[indene-1,5′-oxazolidine]-2′,4′-dione.

### Example 770B

### N-(4-fluorobenzyl)-2-{7-[(methylcarbamoyl)amino]-2′,4′-dioxo-1H,3′H-spiro[isochromene-4,5′-[1,3]oxazolidin]-3′-yl}-N-[(2S)-1,1,1-trifluoropropan-2-yl]acetamide

An oven-dried tube, which was equipped with a magnetic stir bar and fitted with a septa, was charged with 15.4 mg of palladium acetate (five-fold excess) and 100 mg of 2-(di-tert-butylphosphino)-2′,4′,6′-triisopropyl-3,6-dimethoxy-1,1′-biphenyl (five-fold excess). The flask was capped with a septa then evacuated and backfilled with nitrogen three times. Next, tetrahydrofuran (6.9 mL) and degassed water (5.0 mg) were added via syringe. After addition of the water, the solution was heated to 110° C. for 4 minutes. The mixture contained the activated catalyst and was used immediately. A second oven-dried tube, equipped with a magnetic stir bar and septa, was charged with methylurea (0.038 g, 0.512 mmol), cesium carbonate (0.156 g, 0.478 mmol) and 2-(7-bromo-2′,4′-dioxospiro[isochroman-4,5′-oxazolidin]-3′-yl)-N-(4-fluorobenzyl)-N—((S)-1,1,1-trifluoropropan-2-yl)acetamide (0.191 g, 0.341 mmol). The vessel was capped, evacuated, and backfilled with nitrogen three times. Next, 1.4 mL of the catalyst solution prepared above was introduced via syringe. The mixture was evacuated and backfilled with nitrogen two more times then stirred at 50° C. After 3 hours, the mixture was cooled to room temperature and filtered through a bed of diatomaceous earth. The filter cake was washed with ethyl acetate (30 mL) and the filtrate concentrated onto silica gel. Purification by silica gel flash chromatography afforded the title compound as a mixture of diastereomers. ¹H NMR (400 MHz, dimethylsulfoxide-d₆) δ ppm 1.27-1.44 (m, 3H), 2.55-2.71 (m, 3H), 3.90-5.53 (m, 9H), 5.97-6.28 (m, 1H), 6.97-7.53 (m, 7H), 8.56-8.87 (m, 1H). MS (ESI⁺) m/z 553 (M+H)⁺.

### Example 771

### N-(4-fluorobenzyl)-2-[7-(1-methyl-1H-pyrazol-4-yl)-2′,4′-dioxo-1H,3′H-spiro[isochromene-4,5′-[1,3]oxazolidin]-3′-yl]-N-[(2S)-1,1,1-trifluoropropan-2-yl]acetamide

The title compound was prepared as described in EXAMPLE 295, substituting 2-(7-bromo-2′,4′-dioxospiro[isochroman-4,5′-oxazolidin]-3′-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, dimethylsulfoxide-d₆) δ ppm 1.28-1.43 (m, 3H), 3.74-3.93 (m, 3H), 3.95-5.53 (m, 9H), 7.06-7.63 (m, 7H), 7.82-7.95 (m, 1H), 8.12-8.23 (m, 1H). LC/MS (ESI⁺) m/z 561 (M+H)⁺.

### Example 772

### N-(4-fluorobenzyl)-2-(7-{1-[2-(methylamino)-2-oxoethyl]-1H-pyrazol-4-yl}-2′,4′-dioxo-1H,3′H-spiro[isochromene-4,5′-[1,3]oxazolidin]-3′-yl)-N-[(2S)-1,1,1-trifluoropropan-2-yl]acetamide

The title compound was prepared as described in EXAMPLE 295, substituting 2-(7-bromo-2′,4′-dioxospiro[isochroman-4,5′-oxazolidin]-3′-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, dimethylsulfoxide-d₆) δ ppm 1.36 (ddd, J=16.3, 6.9, 4.0 Hz, 3H), 2.53-2.70 (m, 3H), 3.94-5.52 (m, 11H), 7.01-7.74 (m, 7H), 7.89-7.96 (m, 1H), 7.97-8.07 (m, 1H), 8.15-8.27 (m, 1H). MS (ESI⁺) m/z 618 (M+H)⁺.

### Example 773

### N-(4-fluorobenzyl)-2-[(3′R,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 295, substituting 2-[(3′R,4S)-5′-bromo-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.36 (d, J=2.7 Hz, 1H), 8.04 (s, 1H), 7.79 (s, 1H), 7.58 (s, 1H), 7.49 (dd, J=7.9, 1.7 Hz, 2H), 7.32 (dd, J=8.5, 5.4 Hz, 2H), 7.21 (d, J=7.9 Hz, 1H), 7.10 (t, J=8.8 Hz, 2H), 5.20 (dp, J=21.7, 7.6, 7.1 Hz, 3H), 4.89-4.07 (m, 6H), 2.90 (m, 1H), 2.66 (d, J=4.9 Hz, 3H), 2.11 (dd, J=13.2, 6.7 Hz, 1H), 1.33 (d, J=7.0 Hz, 3H). MS (ESI⁺) m/z 617 (M+H)⁺.

### Example 774

### 2-[(3R)-6-amino-1,1-dioxido-2′,5′-dioxo-1′H-spiro[1-benzothiophene-3,4′-imidazolidin]-1′-yl]-N-(4-fluorobenzyl)-N-[(2S)-1,1,1-trifluoropropan-2-yl]acetamide

The diastereomers of 2-(6-amino-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide were separated by chiral prep-SFC (Chiralcel OD-H column) to afford 2-[(3R)-6-amino-1,1-dioxido-2′,5′-dioxo-1′H-spiro[1-benzothiophene-3,4′-imidazolidin]-1′-yl]-N-(4-fluorobenzyl)-N-[(2S)-1,1,1-trifluoropropan-2-yl]acetamide (eluted first) and 2-[(3S)-6-amino-1,1-dioxido-2′,5′-dioxo-1′H-spiro[1-benzothiophene-3,4′-imidazolidin]-1′-yl]-N-(4-fluorobenzyl)-N-[(2S)-1,1,1-trifluoropropan-2-yl]acetamide (eluted second). The assignments of absolute configuration at the spiro center were made based on relative potencies of the two diastereomers and the known SAR of the hydantoin series. ¹H NMR (400 MHz, DMSO-d₆, 120° C.) δ ppm 8.72 (s, 1H), 7.32 (dd, J=8.5, 5.3 Hz, 2H), 7.25 (d, J=8.5 Hz, 1H), 7.11 (t, J=8.8 Hz, 2H), 6.93 (dd, J=8.5, 2.2 Hz, 1H), 6.82 (d, J=2.1 Hz, 1H), 5.60 (brs, 2H), 5.23-5.09 (m, 1H), 4.81 (d, J=17.6 Hz, 1H), 4.59 (d, J=17.6 Hz, 1H), 4.47 (d, J=16.8 Hz, 1H), 4.25 (d, J=16.7 Hz, 1H), 3.82 (d, J=14.2 Hz, 1H), 3.64 (d, J=14.3 Hz, 1H), 1.34 (d, J=7.0 Hz, 3H). MS (ESI⁺) m/z 529 (M+H)⁺.

### Example 775

### N-(4-fluorobenzyl)-N-{1-[2-(methylamino)-2-oxoethyl]-1H-pyrazol-4-yl}-2-{(1R)-5-[(methylcarbamoyl)amino]-2′,4′-dioxo-2,3-dihydro-3′H-spiro[indene-1,5′-[1,3]oxazolidin]-3′-yl}acetamide

### Example 775A

### 2-(4-((4-fluorobenzyl)amino)-1H-pyrazol-1-yl)-N-methylacetamide

The title compound was prepared as described in EXAMPLE 283A, substituting 2-(4-amino-1H-pyrazol-1-yl)-N-methylacetamide for (S)-1-cyclopropylethanamine and 4-fluorobenzaldehyde for benzaldehyde.

### Example 775B

### N-(4-fluorobenzyl)-N-{1-[2-(methylamino)-2-oxoethyl]-1H-pyrazol-4-yl}-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and 2-(4-((4-fluorobenzyl)amino)-1H-pyrazol-1-yl)-N-methylacetamide for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆, 120° C.) δ ppm 8.28 (brs, 1H), 7.78 (s, 1H), 7.53-7.43 (m, 2H), 7.39 (s, 1H), 7.30-7.20 (m, 4H), 7.12-7.02 (m, 2H), 5.89 (brs, 1H), 4.85-4.75 (m, 2H), 4.69 (s, 2H), 4.36-4.17 (m, 2H), 3.20-2.97 (m, 2H), 2.69-2.59 (m, 7H), 2.51-2.41 (m, 1H). MS (ESI⁺) m/z 578 (M+H)⁺.

### Example 776

### N-[1-(cyanomethyl)-1H-pyrazol-4-yl]-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

### Example 776A

### 2-(4-((4-fluorobenzyl)amino)-1H-pyrazol-1-yl)acetonitrile

The title compound was prepared as described in EXAMPLE 283A, substituting 2-(4-amino-1H-pyrazol-1-yl)acetonitrile for (S)-1-cyclopropylethanamine and 4-fluorobenzaldehyde for benzaldehyde.

### Example 776B

### N-(4-fluorobenzyl)-N-{1-[2-(methylamino)-2-oxoethyl]-1H-pyrazol-4-yl}-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and 2-(4-((4-fluorobenzyl)amino)-1H-pyrazol-1-yl)acetonitrile for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆, 120° C.) δ ppm 8.26 (brs, 1H), 7.92 (s, 1H), 7.54 (s, 1H), 7.48 (brs, 1H), 7.28-7.19 (m, 4H), 7.12-7.03 (m, 2H), 5.84 (brs, 1H), 5.33 (s, 2H), 4.83 (brs, 2H), 4.42-4.14 (m, 2H), 3.18-2.93 (m, 2H), 2.71-2.61 (m, 4H), 2.50-2.41 (m, 1H). MS (ESI⁺) m/z 546 (M+H)⁺.

### Example 777

### tert-butyl 3-{1-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]ethyl}azetidine-1-carboxylate

### Example 777A

### tert-butyl 3-(1-((4-fluorobenzyl)amino)ethyl)azetidine-1-carboxylate

The title compound was prepared as described in EXAMPLE 283A, substituting (4-fluorophenyl)methanamine for (S)-1-cyclopropylethanamine and tert-butyl 3-acetylazetidine-1-carboxylate for benzaldehyde.

### Example 777B

### N-(4-fluorobenzyl)-N-{1-[2-(methylamino)-2-oxoethyl]-1H-pyrazol-4-yl}-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and tert-butyl 3-(1-((4-fluorobenzyl)amino)ethyl)azetidine-1-carboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆, 120° C.) δ ppm 8.37-8.22 (m, 1H), 7.49 (s, 1H), 7.40-7.17 (m, 4H), 7.11 (t, J=8.6 Hz, 2H), 5.94-5.81 (m, 1H), 4.65-4.33 (m, 5H), 3.80 (t, J=8.4 Hz, 1H), 3.60-3.36 (m, 4H), 3.20-3.06 (m, 1H), 3.06-2.95 (m, 1H), 2.79-2.61 (m, 4H), 2.53-2.42 (m, 1H), 1.36 (d, J=2.4 Hz, 9H), 1.10 (d, J=6.7 Hz, 3H). MS (ESI⁺) m/z 646 (M+Na)⁺.

### Example 779

### 4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclohexanecarboxylic acid

The title compound was prepared as described in EXAMPLE 302, substituting tert-butyl 4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclohexanecarboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, CD₃OD) δ 7.52 (s, 1H), 7.43-7.32 (m, 2H), 7.26-7.07 (m, 3H), 7.00 (td, J=8.7, 4.5 Hz, 1H), 4.71-4.55 (m, 3H), 4.47-4.25 (m, 2H), 3.91-3.79 (m, 1H), 3.24-3.10 (m, 1H), 3.06 (ddd, J=17.0, 8.9, 4.6 Hz, 1H), 2.82-2.69 (m, 5H), 2.64-2.46 (m, 2H), 2.24-2.14 (m, 1H), 2.09-1.94 (m, 1H), 1.88-1.40 (m, 6H). MS (ESI⁺) m/z 567 (M+H)⁺.

### Example 780

### ethyl ({(3S)-3-[(4-fluorobenzyl) {[(1R)-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]acetyl}amino]pyrrolidin-1-yl}sulfonyl)carbamate

The title compound was prepared as described in EXAMPLE 295, substituting ethyl ({(3R)-3-[{[(1R)-5-bromo-2′,4′-dioxo-2,3-dihydro-3′H-spiro[indene-1,5′-[1,3]oxazolidin]-3′-yl]acetyl}(4-fluorobenzyl)amino]pyrrolidin-1-yl}sulfonyl)carbamate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (500 MHz, DMSO-d₆) δ 8.19 (d, J=10.3 Hz, 1H), 7.94 (d, J=9.0 Hz, 1H), 7.63 (s, 1H), 7.56 (t, J=8.9 Hz, 1H), 7.46 (dd, J=19.5, 8.1 Hz, 1H), 7.34 (dd, J=8.5, 5.1 Hz, 1H), 7.30-7.19 (m, 2H), 7.15 (t, J=8.7 Hz, 1H), 4.95-4.81 (m, 1H), 4.78 (d, J=5.0 Hz, 2H), 4.74 (d, J=6.5 Hz, 2H), 4.60 (s, 1H), 4.47-4.34 (m, 1H), 4.12 (p, J=7.3 Hz, 2H), 4.03 (q, J=7.1 Hz, 1H), 3.78-3.65 (m, 1H), 3.61-3.53 (m, 1H), 3.31 (dt, J=14.9, 7.9 Hz, 1H), 3.24-3.13 (m, 2H), 3.09 (dt, J=10.5, 6.4 Hz, 1H), 2.69 (dd, J=14.9, 7.5 Hz, 1H), 2.63 (t, J=3.9 Hz, 3H), 2.55 (dd, J=8.6, 3.9 Hz, 1H), 1.99 (m, 1H), 1.17 (t, J=7.1 Hz, 3H). MS (ESI⁺) m/z 726 (M+H)⁺.

### Example 781

### ethyl ({(3S)-3-[{[(1R)-5-{1-[2-(dimethylamino)-2-oxoethyl]-1H-pyrazol-4-yl}-2′,4′-dioxo-2,3-dihydro-3′H-spiro[indene-1,5′-[1,3]oxazolidin]-3′-yl]acetyl}(4-fluorobenzyl)amino]pyrrolidin-1-yl}sulfonyl)carbamate

The title compound was prepared as described in EXAMPLE 295, substituting ethyl ({(3R)-3-[{[(1R)-5-bromo-2′,4′-dioxo-2,3-dihydro-3′H-spiro[indene-1,5′-[1,3]oxazolidin]-3′-yl]acetyl}(4-fluorobenzyl)amino]pyrrolidin-1-yl}sulfonyl)carbamate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-N,N-dimethyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.12 (d, J=8.7 Hz, 1H), 7.92 (d, J=7.2 Hz, 1H), 7.62 (s, 1H), 7.55 (t, J=8.2 Hz, 1H), 7.46 (dd, J=15.8, 8.1 Hz, 1H), 7.34 (dd, J=8.6, 5.3 Hz, 1H), 7.29-7.19 (m, 2H), 7.15 (t, J=8.7 Hz, 1H), 5.12 (d, J=4.1 Hz, 2H), 4.95-4.82 (m, 1H), 4.76 (d, J=20.4 Hz, 2H), 4.60 (s, 1H), 4.41 (q, J=16.9 Hz, 1H), 4.12 (p, J=7.0 Hz, 2H), 3.78-3.52 (m, 3H), 3.51-3.25 (m, 2H), 3.15-3.07 (m, 2H), 3.04 (d, J=2.8 Hz, 3H), 2.86 (d, J=2.5 Hz, 3H), 2.76-2.52 (m, 2H), 1.98 (q, J=10.0, 9.0 Hz, 1H), 1.21 (q, J=6.8 Hz, 3H). MS (ESI⁺) m/z 740 (M+H)⁺.

### Example 782

### tert-butyl {4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]piperidin-1-yl}acetate

The title compound was prepared as described in EXAMPLE 832A, substituting N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-(piperidin-4-yl)acetamide for 5′-bromo-2′,3′-dihydrospiro[imidazolidine-4,1′-indene]-2,5-dione. ¹H NMR (501 MHz, DMSO-d₆) δ 7.56 (s, 1H), 7.38 (dd, J=8.4, 5.3 Hz, 1H), 7.35-7.18 (m, 4H), 7.12 (t, J=8.7 Hz, 1H), 4.70 (d, J=11.2 Hz, 2H), 4.64-4.51 (m, 1H), 4.37 (d, J=3.1 Hz, 1H), 3.19-3.06 (m, 4H), 2.86-2.76 (m, 2H), 2.65 (q, J=5.5, 4.3 Hz, 1H), 2.63 (t, J=3.9 Hz, 3H), 2.50 (dq, J=6.2, 4.2, 3.3 Hz, 1H), 2.32 (t, J=11.4 Hz, 1H), 2.24-2.13 (m, 1H), 1.74-1.43 (m, 5H), 1.41 (s, 9H). MS (ESI⁺) m/z 638 (M+H)⁺.

### Example 783

### ethyl ({(3R)-3-[(4-fluorobenzyl) {[(1R)-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]acetyl}amino]piperidin-1-yl}sulfonyl)carbamate

The title compound was prepared as described in EXAMPLE 295, substituting ethyl ({(3R)-3-[{[(1R)-5-bromo-2′,4′-dioxo-2,3-dihydro-3′H-spiro[indene-1,5′-[1,3]oxazolidin]-3′-yl]acetyl}(4-fluorobenzyl)amino]piperidin-1-yl}sulfonyl)carbamate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.20 (d, J=6.1 Hz, 1H), 7.95 (d, J=6.1 Hz, 1H), 7.63 (s, 1H), 7.57 (dd, J=14.9, 8.2 Hz, 1H), 7.48 (dd, J=12.4, 7.9 Hz, 1H), 7.38 (dd, J=8.5, 5.4 Hz, 1H), 7.29-7.19 (m, 2H), 7.14 (t, J=8.7 Hz, 1H), 4.83-4.69 (m, 3H), 4.65-4.47 (m, 2H), 4.33-4.20 (m, 1H), 4.15-3.93 (m, 2H), 3.53 (m, 2H), 3.28-3.13 (m, 1H), 3.08 (ddd, J=17.7, 9.2, 3.5 Hz, 1H), 2.90 (dd, J=21.4, 10.7 Hz, 1H), 2.71 (dt, J=17.0, 9.7 Hz, 1H), 2.63 (t, J=3.3 Hz, 3H), 2.58-2.51 (m, 1H), 1.74 (d, J=11.9 Hz, 2H), 1.64 (m, 3H), 1.31 (q, J=7.6, 7.1 Hz, 1H), 1.19 (t, J=7.0 Hz, 3H). MS (ESI⁺) m/z 740 (M+H)⁺.

### Example 784

### ethyl ({(3R)-3-[(4-fluorobenzyl){[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]piperidin-1-yl}sulfonyl)carbamate

The title compound was prepared as described in EXAMPLE 295, substituting ethyl ({(3R)-3-[{[(1R)-5-bromo-2′,4′-dioxo-2,3-dihydro-3′H-spiro[indene-1,5′-[1,3]oxazolidin]-3′-yl]acetyl}(4-fluorobenzyl)amino]piperidin-1-yl}sulfonyl)carbamate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.18 (d, J=6.8 Hz, 1H), 7.90 (d, J=6.0 Hz, 1H), 7.61 (s, 1H), 7.59-7.53 (m, 1H), 7.47 (dd, J=12.9, 8.0 Hz, 1H), 7.38 (dd, J=8.5, 5.3 Hz, 1H), 7.31-7.19 (m, 2H), 7.14 (t, J=8.9 Hz, 1H), 4.74 (s, 1H), 4.65-4.47 (m, 2H), 4.34-4.21 (m, 1H), 4.12-4.03 (m, 3H), 3.86 (d, J=3.2 Hz, 3H), 3.67-3.48 (m, 2H), 3.27-3.02 (m, 2H), 2.92 (dt, J=22.2, 11.5 Hz, 1H), 2.81-2.63 (m, 2H), 2.62-2.51 (m, 1H), 1.79-1.44 (m, 4H), 1.19 (t, J=7.1 Hz, 3H). MS (ESI⁺) m/z 683 (M+H)⁺.

### Example 785

### {4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]piperidin-1-yl}acetic acid

The title compound was prepared as described in EXAMPLE 302, substituting tert-butyl {4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]piperidin-1-yl}acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 7.57-7.50 (m, 1H), 7.41-7.30 (m, 2H), 7.25 (td, J=9.0, 8.4, 4.3 Hz, 3H), 7.13 (t, J=8.8 Hz, 1H), 4.83 (d, J=19.3 Hz, 2H), 4.61 (m, 2H), 4.38 (d, J=25.3 Hz, 3H), 4.07 (d, J=13.4 Hz, 2H), 3.62 (t, J=12.8 Hz, 2H), 3.20-2.88 (m, 2H), 2.64 (d, J=3.3 Hz, 3H), 2.59 (d, J=8.4 Hz, 1H), 2.49 (d, J=1.9 Hz, 1H), 2.21 (d, J=13.5 Hz, 2H), 1.94-1.64 (m, 2H). MS (ESI⁺) m/z 582 (M+H)⁺.

### Example 786

### 2-{2,5-dioxo-5′-[(2,2,2-trifluoroethyl)amino]-2′,3′-dihydro-1H-spiro[imidazolidine-4,1′-inden]-1-yl}-N-(4-fluorobenzyl)-N-[(2S)-1, 1,1-trifluoropropan-2-yl]acetamide

### Example 786A

### 2-(5′-bromo-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide

The title compound was prepared as described in EXAMPLE 281F, substituting 5′-bromo-2′,3′-dihydrospiro[imidazolidine-4,1′-indene]-2,5-dione for (R)-5-bromo-2,3-dihydrospiro[indene-1,5′-oxazolidine]-2′,4′-dione and (S)-2-bromo-N-(4-fluorobenzyl)-N-(1,1,1-trifluoropropan-2-yl)acetamide for (S)-2-bromo-N-(4-fluorobenzyl)-N-(1,1,1-trifluoropropan-2-yl)acetamide.

### Example 786B

### 2-{2,5-dioxo-5′-[(2,2,2-trifluoroethyl)amino]-2′,3′-dihydro-1H-spiro[imidazolidine-4,1′-inden]-1-yl}-N-(4-fluorobenzyl)-N-[(2S)-1, 1,1-trifluoropropan-2-yl]acetamide

The title compound was prepared as described in EXAMPLE 284D, substituting 2-(5′-bromo-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide and 2,2,2-trifluoroethylamine for diphenylmethanimine. ¹H NMR (400 MHz, dimethylsulfoxide-d₆) δ ppm 1.30 (dd, J=25.8, 6.9 Hz, 3H), 2.02-2.22 (m, 1H), 2.40-2.48 (m, 1H), 2.79-2.99 (m, 2H), 3.77-3.98 (m, 2H), 3.98-4.49 (m, 2H), 4.60-5.51 (m, 3H), 6.30 (t, J=6.9 Hz, 1H), 6.53-6.70 (m, 2H), 6.89-7.49 (m, 5H), 8.59-8.74 (m, 1H). MS (ESI⁺) m/z 561 (M+H)⁺.

### Example 787

### 2-[(4S)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

### Example 787A

### (S)—N-(2,5-dioxo-2′,3′-dihydrospiro[imidazolidine-4,1′-inden]-5′-yl)acetamide

The diastereomeric mixture of N-(2,5-dioxo-2′,3′-dihydrospiro[imidazolidine-4,1′-inden]-5′-yl)acetamide was separated by chiral prep-SFC (Chiralpak AD-H) to afford (R)—N-(2,5-dioxo-2′,3′-dihydrospiro[imidazolidine-4,1′-inden]-5′-yl)acetamide (eluted first) and (S)—N-(2,5-dioxo-2′,3′-dihydrospiro[imidazolidine-4,1′-inden]-5′-yl)acetamide (eluted second).

### Example 787B

### 2-[(4S)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 281F, substituting (S)—N-(2,5-dioxo-2′,3′-dihydrospiro[imidazolidine-4,1′-inden]-5′-yl)acetamide for (R)-5-bromo-2,3-dihydrospiro[indene-1,5′-oxazolidine]-2′,4′-dione. ¹H NMR (400 MHz, DMSO-d₆) δ 9.49 (s, 1H), 8.41-8.09 (m, 1H), 7.54 (d, J=1.8 Hz, 1H), 7.46-7.17 (m, 3H), 7.16-6.90 (m, 3H), 5.16 (p, J=7.9 Hz, 1H), 4.86-4.11 (m, 4H), 3.00 (t, J=7.2 Hz, 2H), 2.55 (dt, J=13.4, 6.7 Hz, 1H), 2.22 (dt, J=13.3, 7.7 Hz, 1H), 1.33 (d, J=7.0 Hz, 3H). MS (ESI⁺) m/z 521 (M+H)⁺.

### Example 788

### 2-[(4R)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 281F, substituting (R)-5′-bromo-2′,3′-dihydrospiro[imidazolidine-4,1′-indene]-2,5-dione for (R)-5-bromo-2,3-dihydrospiro[indene-1,5′-oxazolidine]-2′,4′-dione. ¹H NMR (400 MHz, DMSO-d₆) δ 8.54 (s, 1H), 7.51 (d, J=1.6 Hz, 1H), 7.42 (dd, J=8.1, 2.0 Hz, 1H), 7.32 (s, 2H), 7.20 (d, J=8.2 Hz, 1H), 7.13 (t, J=8.7 Hz, 2H), 5.19 (s, 1H), 4.89-4.11 (m, 4H), 3.05 (t, J=7.3 Hz, 2H), 2.56 (dt, J=13.3, 6.6 Hz, 1H), 2.24 (dt, J=13.4, 7.8 Hz, 1H), 1.33 (d, J=7.1 Hz, 3H). MS (ESI⁺) m/z 542 (M+H)⁺.

### Example 789

### 2-{(3′R,4S)-5′-[1-(2-amino-2-oxoethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide

The title compound was prepared as described in EXAMPLE 295, substituting 2-[(3′R,4S)-5′-bromo-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.60 (d, J=3.4 Hz, 1H), 8.11 (s, 1H), 7.86 (s, 1H), 7.77-7.61 (m, 2H), 7.39-7.25 (m, 3H), 7.10 (t, J=8.9 Hz, 2H), 6.89 (s, 2H), 6.12 (ddd, J=57.7, 6.8, 4.5 Hz, 1H), 5.27-5.02 (m, 1H), 4.89-4.14 (m, 6H), 3.05 (ddd, J=14.4, 12.5, 6.8 Hz, 1H), 2.45-2.17 (m, 1H), 1.34 (d, J=7.0 Hz, 3H). MS (ESI⁺) m/z 603 (M−H)⁺.

### Example 790

### 2-(5′-bromo-6′-fluoro-3′-hydroxy-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

The title compound was prepared as described in EXAMPLE 570A, substituting 2-(5′-bromo-6′-fluoro-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide. ¹H NMR (400 MHz, DMSO-d₆) δ 8.89 (dd, J=20.5, 14.0 Hz, 1H), 7.69 (t, J=7.7 Hz, 1H), 7.43-7.04 (m, 5H), 5.77-5.67 (m, 1H), 5.45-5.33 (m, 0.6H), 5.27 (dd, J=12.6, 6.1 Hz, 1H), 5.07 (d, J=6.3 Hz, 0.4H), 4.99-4.62 (m, 2H), 4.54-4.31 (m, 1.6H), 4.21-3.98 (m, 1.4H), 2.61-2.50 (m, 1H), 2.32 (ddd, J=13.2, 10.2, 6.4 Hz, 1H), 1.33 (dt, J=13.8, 6.6 Hz, 3H). MS (ESI⁻) m/z 574 (M−H)⁻.

### Example 791

### N-(4-fluorobenzyl)-2-(6′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide

The title compound was prepared as described in EXAMPLE 295, substituting 2-(5′-bromo-6′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (501 MHz, DMSO-d₆) δ 8.95-8.80 (m, 1H), 8.14 (s, 1H), 8.04 (s, 1H), 7.94 (s, 1H), 7.69 (d, J=7.1 Hz, 1H), 7.43-7.33 (m, 1.3H), 7.22 (t, J=8.7 Hz, 2H), 7.18-7.04 (m, 1.7H), 5.51-5.27 (m, 0.7H), 5.07 (d, J=6.2 Hz, 0.3H), 4.98-4.63 (m, 4H), 4.39 (td, J=17.2, 7.3 Hz, 1.3H), 4.08 (dd, J=16.9, 9.2 Hz, 0.7H), 3.03 (d, J=6.9 Hz, 2H), 2.62 (d, J=4.5 Hz, 2H), 2.61-2.54 (m, 1H), 2.26 (tt, J=13.0, 7.8 Hz, 1H), 1.44-1.19 (m, 3H). MS (ESI⁺) m/z 619 (M+H)⁺.

### Example 792

### 2-{5′-[1-(2-amino-2-oxoethyl)-1H-pyrazol-4-yl]-6′-fluoro-2,5-dioxo-2′,3′-dihydro-H-spiro[imidazolidine-4,1′-inden]-1-yl}-N-(4-fluorobenzyl)-N-[(2S)-1,1,1-trifluoropropan-2-yl]acetamide

The title compound was prepared as described in EXAMPLE 295, substituting 2-(5′-bromo-6′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (500 MHz, DMSO-d₆) δ 8.87 (d, J=13.6 Hz, 1H), 8.13 (s, 1H), 7.93 (s, 1H), 7.68 (d, J=7.1 Hz, 1H), 7.53 (s, 1H), 7.45-7.34 (m, 1.3H), 7.23 (dd, J=20.2, 11.5 Hz, 3H), 7.16-7.07 (m, 1.7H), 5.48-5.28 (m, 0.7H), 5.07 (d, J=6.8 Hz, 0.3H), 4.95-4.64 (m, 4H), 4.39 (td, J=17.2, 7.4 Hz, 1.3H), 4.08 (dd, J=16.9, 9.6 Hz, 0.7H), 3.03 (d, J=7.1 Hz, 2H), 2.58 (dt, J=13.5, 10.1 Hz, 1H), 2.32-2.17 (m, 1H), 1.40-1.25 (m, 3H). MS (ESI⁺) m/z 605 (M+H)⁺.

### Example 793

### N-(4-fluorobenzyl)-2-{6′-fluoro-5′-[1-(2-hydroxyethyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide

The title compound was prepared as described in EXAMPLE 295, substituting 2-(5′-bromo-6′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)ethanol for 5-(4,4,5,5-tetramethyl-1,3,2-dioxaborolan-2-yl)pyridin-2-ol. ¹H NMR (400 MHz, DMSO-d₆) δ 8.85 (d, J=10.8 Hz, 1H), 8.11 (s, 1H), 7.90 (s, 1H), 7.66 (d, J=7.1 Hz, 1H), 7.44-7.30 (m, 1H), 7.20 (t, J=8.6 Hz, 2H), 7.10 (dd, J=12.7, 7.2 Hz, 2H), 5.42-5.29 (m, 0.7H), 5.06 (s, 0.3H), 4.95-4.61 (m, 2H), 4.45-4.29 (m, 1.3H), 4.17 (t, J=5.1 Hz, 2H), 4.07 (dd, J=17.0, 6.9 Hz, 0.3H), 3.75 (t, J=5.4 Hz, 2H), 3.00 (s, 2H), 2.55 (dd, J=14.1, 6.8 Hz, 1H), 2.25 (t, J=14.6 Hz, 1H), 1.31 (dd, J=25.1, 6.8 Hz, 3H). MS (ESI⁺) m/z 592 (M+H)⁺.

### Example 794

### N-(4-fluorobenzyl)-2-[6′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 295, substituting 2-(5′-bromo-6′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.85 (d, J=10.5 Hz, 1H), 8.11 (s, 1H), 7.88 (s, 1H), 7.64 (d, J=7.1 Hz, 1H), 7.46-7.30 (m, 1.4H), 7.20 (t, J=8.7 Hz, 2H), 7.10 (dt, J=9.6, 4.0 Hz, 1.6H), 5.43-5.29 (m, 0.6H), 5.06 (s, 0.4H), 4.79 (ddd, J=44.7, 29.7, 18.8 Hz, 2H), 4.45-4.30 (m, 1.4H), 4.07 (dd, J=16.9, 6.1 Hz, 0.6H), 3.87 (s, 3H), 3.01 (d, J=6.4 Hz, 2H), 2.56 (dt, J=11.9, 5.9 Hz, 1H), 2.31-2.16 (m, 1H), 1.31 (dd, J=25.5, 6.7 Hz, 3H). MS (ESI⁻) m/z 560 (M−H)⁻.

### Example 795

### 2-(5′-bromo-3′,6′-difluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

The title compound was prepared as described in EXAMPLE 514, substituting 2-(5′-bromo-6′-fluoro-3′-hydroxy-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide. ¹H NMR (400 MHz, DMSO-d₆) δ 9.14 (d, J=11.2 Hz, 1H), 7.98 (d, J=6.1 Hz, 1H), 7.44-7.27 (m, 2H), 7.19 (dd, J=11.6, 5.7 Hz, 1.3H), 7.09 (dd, J=17.9, 8.8 Hz, 0.0.7H), 6.10 (d, J=57.7 Hz, 1H), 5.42-5.26 (m, 0.7H), 5.03 (s, 0.3H), 4.94-4.63 (m, 2H), 4.39 (dt, J=26.4, 13.3 Hz, 1.3H), 4.06 (dd, J=16.9, 14.4 Hz, 0.7H), 3.06 (ddd, J=18.4, 12.1, 6.9 Hz, 1H), 2.46-2.28 (m, 1H), 1.37-1.23 (m, 3H). MS (ESI⁺) m/z 580 (M+H)⁺.

### Example 796

### tert-butyl 4-[1-(2-{(4-fluorobenzyl) [(2S)-1,1,1-trifluoropropan-2-yl]amino}-2-oxoethyl)-2,5-dioxo-2′,3′-dihydrospiro[imidazolidine-4,1′-inden]-4′-yl]-3,6-dihydropyridine-1(2H)-carboxylate

The title compound was prepared as described in EXAMPLE 295, substituting 2-(4′-bromo-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and tert-butyl 4-(4,4,5,5-tetramethyl-1,3,2-dioxaborolan-2-yl)-5,6-dihydropyridine-1(2H)-carboxylate for 5-(4,4,5,5-tetramethyl-1,3,2-dioxaborolan-2-yl)pyridin-2-ol. ¹H NMR (400 MHz, DMSO-d₆) δ 8.78 (d, J=10.5 Hz, 1H), 7.48-6.90 (m, 7H), 5.78 (s, 1H), 5.42-5.22 (m, 0.7H), 5.04 (s, 0.3H), 4.77 (ddd, J=41.4, 38.2, 19.1 Hz, 2H), 4.34 (dd, J=22.4, 11.6 Hz, 1.3H), 4.05 (dd, J=11.1, 5.8 Hz, 0.7H), 3.92 (d, J=26.6 Hz, 2H), 3.65-3.42 (m, 2H), 3.08-2.96 (m, 2H), 2.44-2.25 (m, 3H), 2.22-2.05 (m, 1H), 1.45-1.21 (m, 12H). MS (ESI⁻) m/z 643.2 (M−H)⁻.

### Example 797

### N-(4-fluorobenzyl)-2-{(4S)-7-[(methylcarbamoyl)amino]-2′,4′-dioxo-1H,3′H-spiro[isochromene-4,5′-[1,3]oxazolidin]-3′-yl}-N-[(2S)-1,1,1-trifluoropropan-2-yl]acetamide

The diastereomeric mixture of N-(4-fluorobenzyl)-2-{7-[(methylcarbamoyl)amino]-2′,4′-dioxo-1H,3′H-spiro[isochromene-4,5′-[1,3]oxazolidin]-3′-yl}-N-[(2S)-1,1,1-trifluoropropan-2-yl]acetamide was purified on chiral prep-SFC (Whelk-01 SS column) to provide N-(4-fluorobenzyl)-2-{(4S)-7-[(methylcarbamoyl)amino]-2′,4′-dioxo-1H,3′H-spiro[isochromene-4,5′-[1,3]oxazolidin]-3′-yl}-N-[(2S)-1,1,1-trifluoropropan-2-yl]acetamide (eluted first) and N-(4-fluorobenzyl)-2-{(4R)-7-[(methylcarbamoyl)amino]-2′,4′-dioxo-1H,3′H-spiro[isochromene-4,5′-[1,3]oxazolidin]-3′-yl}-N-[(2S)-1,1,1-trifluoropropan-2-yl]acetamide (eluted second). ¹H NMR (501 MHz, dimethylsulfoxide-d₆) δ ppm 1.36 (dd, J=15.7, 6.9 Hz, 3H), 2.55-2.70 (m, 3H), 3.95-4.59 (m, 4H), 4.64-5.52 (m, 5H), 6.10-6.32 (m, 1H), 7.07-7.47 (m, 7H), 8.70-8.96 (m, 1H). MS (ESI⁺) m/z 553 (M+H)⁺.

### Example 798

### 2-[2,5-dioxo-4′-(1,2,3,6-tetrahydropyridin-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 302, substituting tert-butyl 4-[1-(2-{(4-fluorobenzyl)[(2S)-1,1,1-trifluoropropan-2-yl]amino}-2-oxoethyl)-2,5-dioxo-2′,3′-dihydrospiro[imidazolidine-4,1′-inden]-4′-yl]-3,6-dihydropyridine-1(2H)-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 8.82 (d, J=13.3 Hz, 3H), 7.46-6.99 (m, 7H), 5.81 (s, 1H), 5.34 (s, 0.6H), 5.05 (s, 0.4H), 4.78 (dt, J=37.8, 19.3 Hz, 2H), 4.39 (d, J=16.1 Hz, 1.4H), 4.07 (d, J=17.3 Hz, 0.6H), 3.74 (s, 2H), 3.40-3.30 (m, 2H), 3.05 (s, 2H), 2.71-2.53 (m, 3H), 2.18 (s, 2H), 1.31 (dd, J=25.8, 6.0 Hz, 3H). MS (ESI⁺) m/z 545 (M+H)⁺.

### Example 799

### N-(4-fluorobenzyl)-2-{(4R)-7-[(methylcarbamoyl)amino]-2′,4′-dioxo-1H,3′H-spiro[isochromene-4,5′-[1,3]oxazolidin]-3′-yl}-N-[(2S)-1,1,1-trifluoropropan-2-yl]acetamide

The diastereomeric mixture of N-(4-fluorobenzyl)-2-{7-[(methylcarbamoyl)amino]-2′,4′-dioxo-1H,3′H-spiro[isochromene-4,5′-[1,3]oxazolidin]-3′-yl}-N-[(2S)-1,1,1-trifluoropropan-2-yl]acetamide was purified by chiral prep-SFC (Whelk-01 SS column) to provide N-(4-fluorobenzyl)-2-{(4S)-7-[(methylcarbamoyl)amino]-2′,4′-dioxo-1H,3′H-spiro[isochromene-4,5′-[1,3]oxazolidin]-3′-yl}-N-[(2S)-1,1,1-trifluoropropan-2-yl]acetamide (eluted first) and N-(4-fluorobenzyl)-2-{(4R)-7-[(methylcarbamoyl)amino]-2′,4′-dioxo-1H,3′H-spiro[isochromene-4,5′-[1,3]oxazolidin]-3′-yl}-N-[(2S)-1,1,1-trifluoropropan-2-yl]acetamide (eluted second). ¹H NMR (501 MHz, dimethylsulfoxide-d₆) δ ppm 1.36 (dd, J=28.5, 6.9 Hz, 3H), 2.57-2.71 (m, 3H), 3.97-5.51 (m, 9H), 6.02-6.30 (m, 1H), 7.02-7.50 (m, 7H), 8.67-8.91 (m, 1H).). MS (ESI⁺) m/z 553 (M+H)⁺.

### Example 800

### 2-{5′-[1-(2-amino-2-oxoethyl)-1H-pyrazol-4-yl]-3′,6′-difluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide

The title compound was prepared as described in EXAMPLE 295, substituting 2-(5′-bromo-3′,6′-difluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (500 MHz, DMSO-d₆) δ 9.12 (d, J=14.4 Hz, 1H), 8.21 (q, J=3.2, 2.7 Hz, 1H), 8.02 (s, 1H), 7.96 (d, J=6.9 Hz, 1H), 7.54 (s, 1H), 7.37 (ddd, J=8.6, 5.4, 2.8 Hz, 1.3H), 7.32-7.17 (m, 3H), 7.11 (dt, J=12.7, 8.8 Hz, 0.7H), 6.14 (ddq, J=57.7, 7.5, 4.1, 3.6 Hz, 1H), 5.44-5.31 (m, 0.7H), 5.10-5.03 (m, 0.3H), 4.94-4.65 (m, 4H), 4.48-4.34 (m, 1.3H), 4.09 (t, J=17.6 Hz, 0.7H), 3.08 (tdd, J=14.3, 7.0, 3.7 Hz, 1H), 2.38 (ddt, J=25.4, 14.6, 4.3 Hz, 1H), 1.32 (ddd, J=30.2, 7.0, 3.0 Hz, 3H). MS (ESI⁺) m/z 623 (M+H)⁺.

### Example 801

### 2-{4-[3′,6′-difluoro-1-(2-{(4-fluorobenzyl) [(2S)-1,1,1-trifluoropropan-2-yl]amino}-2-oxoethyl)-2,5-dioxo-2′,3′-dihydro-1H-spiro[imidazolidine-4,1′-inden]-5′-yl]-1H-pyrazol-1-yl}-N,N-dimethylacetamide

The title compound was prepared as described in EXAMPLE 295, substituting 2-(5′-bromo-3′,6′-difluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N,N-dimethyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d6) δ 9.13 (d, J=11.5 Hz, 1H), 8.15 (d, J=2.9 Hz, 1H), 8.05-7.91 (m, 2H), 7.65-7.43 (m, 1.4H), 7.38 (ddd, J=8.1, 5.4, 2.3 Hz, 1H), 7.33-7.17 (m, 2H), 7.12 (q, J=9.2 Hz, 0.6H), 6.28-5.97 (m, 1H), 5.47-5.30 (m, 0.6H), 5.18 (s, 2H), 5.10-5.04 (m, 0.4H), 4.97-4.65 (m, 2H), 4.53-4.33 (m, 1.4H), 4.10 (dd, J=17.0, 14.7 Hz, 0.6H), 3.15-3.06 (m, 1H), 3.05 (s, 3H), 2.86 (s, 3H), 2.47-2.29 (m, 1H), 1.33 (dd, J=26.9, 7.0 Hz, 3H). MS (ESI⁺) m/z 651 (M+H)⁺.

### Example 802

### 2-[3′,6′-difluoro-5′-(1-methyl-1H-pyrazol-4-yl)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 295, substituting 2-(5′-bromo-3′,6′-difluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d6) δ 9.18-9.07 (m, 1H), 8.22 (d, J=2.7 Hz, 1H), 8.03-7.90 (m, 2H), 7.38 (ddd, J=8.5, 5.4, 2.8 Hz, 1.3H), 7.32-7.17 (m, 3H), 7.12 (dt, J=10.7, 8.8 Hz, 0.7H), 6.14 (ddt, J=57.7, 7.0, 3.5 Hz, 1H), 5.38 (p, J=7.8 Hz, 0.7H), 5.10-5.02 9 m, 0.3H), 4.97-4.63 (m, 2H), 4.41 (tdd, J=13.9, 8.1, 4.9 Hz, 1.3H), 4.10 (dd, J=17.0, 13.1 Hz, 0.7H), 3.90 (d, J=1.9 Hz, 3H), 3.09 (dddd, J=17.5, 14.5, 6.6, 2.9 Hz, 1H), 2.47-2.30 (m, 1H), 1.33 (ddd, J=24.9, 7.0, 2.2 Hz, 3H). MS (ESI) m/z 580 (M+H)⁺.

### Example 803

### 2-{3′,6′-difluoro-5′-[1-(2-hydroxyethyl)-H-pyrazol-4-yl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide

The title compound was prepared as described in EXAMPLE 295, substituting 2-(5′-bromo-3′,6′-difluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)ethanol for 5-(4,4,5,5-tetramethyl-1,3,2-dioxaborolan-2-yl)pyridin-2-ol. ¹H NMR (400 MHz, DMSO-d₆) δ 9.12 (d, J=11.5 Hz, 1H), 8.22 (s, 1H), 8.04-7.92 (m, 2H), 7.37 (ddd, J=8.3, 5.4, 2.5 Hz, 1.3H), 7.32-7.17 (m, 3H), 7.12 (q, J=9.3 Hz, 0.7H), 6.14 (dq, J=58.3, 5.6, 4.5 Hz, 1H), 5.37 (p, J=8.0 Hz, 1H), 5.13-5.02 (m, 0.3H), 4.95-4.65 (m, 2H), 4.53-4.34 (m, 1.3H), 4.20 (t, J=5.7 Hz, 2H), 4.09 (dd, J=17.0, 13.8 Hz, 0.7H), 3.77 (td, J=5.6, 1.9 Hz, 2H), 3.08 (dqd, J=14.3, 6.6, 3.0 Hz, 1H), 2.47-2.31 (m, 1H), 1.33 (dd, J=25.1, 5.4 Hz, 3H). MS (ESI⁻) m/z 608 (M−H)⁻.

### Example 804

### N-(4-fluorobenzyl)-2-[(4R)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 295, substituting 2-[(4R)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.49 (s, 1H), 8.01 (s, 1H), 7.76 (s, 1H), 7.47-7.07 (m, 7H), 5.21 (s, 1H), 4.92-4.05 (m, 4H), 3.85 (s, 3H), 3.05 (t, J=7.2 Hz, 2H), 2.57 (dt, J=13.3, 6.6 Hz, 1H), 2.23 (dt, J=13.4, 7.7 Hz, 1H), 1.33 (d, J=7.0 Hz, 3H). MS (ESI⁺) m/z 544 (M+H)⁺.

### Example 805

### tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]piperidine-1-carboxylate

### Example 805A

### (R)-tert-butyl 3-((4-fluorobenzyl)amino)piperidine-1-carboxylate

The title compound was prepared as described in EXAMPLE 283A, substituting (R)-tert-butyl 3-aminopiperidine-1-carboxylate for (S)-1-cyclopropylethanamine and 4-fluorobenzaldehyde for benzaldehyde.

### Example 805B

### tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]piperidine-1-carboxylate

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and (R)-tert-butyl 3-((4-fluorobenzyl)amino)piperidine-1-carboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, methanol-d₄) δ 7.52 (s, 1H), 7.39 (ddd, J=14.5, 8.4, 5.4 Hz, 2H), 7.21 (s, 1H), 7.15 (q, J=8.5, 8.0 Hz, 1H), 7.04 (td, J=9.5, 9.0, 5.1 Hz, 2H), 4.68 (s, 3H), 4.56-4.39 (m, 1H), 4.15-3.89 (m, 2H), 3.81 (dd, J=16.2, 3.1 Hz, 2H), 3.19 (ddd, J=15.5, 9.8, 5.7 Hz, 1H), 3.06 (ddd, J=16.2, 8.9, 4.3 Hz, 1H), 2.83 (d, J=20.1 Hz, 1H), 2.76 (d, J=1.9 Hz, 3H), 2.55 (dddd, J=14.9, 11.9, 8.0, 3.6 Hz, 2H), 2.05-1.86 (m, 2H), 1.84-1.65 (m, 2H), 1.47-1.41 (m, 9H). MS (ESI⁺) m/z 624 (M+H)⁺.

### Example 806

### tert-butyl (3S)-3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]piperidine-1-carboxylate

### Example 806A

### (S)-tert-butyl 3-((4-fluorobenzyl)amino)piperidine-1-carboxylate

The title compound was prepared as described in EXAMPLE 283A, substituting (S)-tert-butyl 3-aminopiperidine-1-carboxylate for (S)-1-cyclopropylethanamine and 4-fluorobenzaldehyde for benzaldehyde.

### Example 806B

### tert-butyl (3S)-3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]piperidine-1-carboxylate

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and (S)-tert-butyl 3-((4-fluorobenzyl)amino)piperidine-1-carboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, methanol-d₄) δ 7.51 (s, 1H), 7.45-7.34 (m, 2H), 7.30-7.07 (m, 3H), 7.02 (t, J=8.6 Hz, 1H), 4.67 (s, 3H), 4.53 (d, J=16.0 Hz, 1H), 4.40 (d, J=16.6 Hz, 1H), 4.15-3.89 (m, 3H), 3.82 (td, J=10.6, 9.8, 5.2 Hz, 1H), 3.18 (qd, J=7.8, 3.2 Hz, 1H), 3.06 (ddt, J=16.3, 8.3, 4.0 Hz, 1H), 2.76 (d, J=3.0 Hz, 3H), 2.66-2.45 (m, 3H), 1.94-1.63 (m, 3H), 1.47-1.37 (m, 9H). MS (ESI⁺) m/z 624 (M+H)⁺.

### Example 807

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-piperidin-3-yl]acetamide

The title compound was prepared as described in EXAMPLE 302, substituting tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]piperidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, methanol-d₄) δ 7.46-7.36 (m, 3H), 7.31 (dd, J=8.4, 1.9 Hz, 1H), 7.16 (t, J=8.7 Hz, 2H), 7.04 (s, 1H), 4.72 (s, 3H), 4.66-4.41 (m, 2H), 4.41-4.25 (m, 1H), 3.28 (d, J=15.8 Hz, 2H), 3.23-3.12 (m, 2H), 3.11-3.01 (m, 1H), 2.85 (t, J=12.8 Hz, 1H), 2.77 (m, 1H), 2.61-2.45 (m, 1H), 1.96 (t, J=12.5 Hz, 3H), 1.90-1.68 (m, 3H). MS (ESI⁺) m/z 524 (M+H)⁺.

### Example 808

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3S)-piperidin-3-yl]acetamide

The title compound was prepared as described in EXAMPLE 302, substituting tert-butyl (3S)-3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]piperidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, methanol-d₄) δ 7.48-7.34 (m, 4H), 7.34-7.23 (m, 1H), 7.16 (t, J=8.7 Hz, 2H), 4.70 (s, 3H), 4.53 (q, J=16.7 Hz, 2H), 4.40-4.23 (m, 1H), 3.26 (d, J=3.7 Hz, 2H), 3.23-3.13 (m, 1H), 3.06 (ddd, J=16.3, 8.6, 3.8 Hz, 1H), 2.86 (td, J=13.0, 3.0 Hz, 1H), 2.77 (d, J=2.6 Hz, 1H), 2.74 (d, J=6.6 Hz, 1H), 2.54 (ddd, J=14.5, 8.2, 3.7 Hz, 1H), 2.07-1.91 (m, 3H), 1.85 (d, J=11.2 Hz, 2H), 1.75 (t, J=14.1 Hz, 1H). MS (ESI⁺) m/z 524 (M+H)⁺.

### Example 809

### N-[4-amino-3-(hydroxymethyl)butan-2-yl]-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 302, substituting tert-butyl 3-{1-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]ethyl}azetidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆, 120° C.) δ ppm 8.31 (brs, 1H), 8.15 (brs, 1H), 7.58-7.47 (m, 2H), 7.43 (brs, 1H), 7.31-7.13 (m, 4H), 5.90 (brs, 1H), 4.30-4.06 (m, 4H), 3.75-3.55 (m, 2H), 3.51-2.80 (m, 5H), 2.70-2.60 (m, 4H), 2.52-2.40 (m, 1H), 2.30-2.18 (m, 1H), 1.32 (d, J=6.6 Hz, 3H). MS (ESI⁺) m/z 542 (M+H)⁺.

### Example 810

### ethyl ({(3S)-3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]piperidin-1-yl}sulfonyl)carbamate

The title compound was prepared as described in EXAMPLE 678, substituting N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3S)-piperidin-3-yl]acetamide for N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide. ¹H NMR (501 MHz, methanol-d₄) δ 7.52 (s, 1H), 7.45-7.34 (m, 3H), 7.28-7.11 (m, 3H), 7.04 (dt, J=23.3, 8.6 Hz, 1H), 4.67 (dt, J=21.9, 5.1 Hz, 3H), 4.57-4.37 (m, 1H), 4.28-4.02 (m, 3H), 3.90-3.57 (m, 2H), 3.26-3.12 (m, 1H), 3.06 (dq, J=12.3, 4.4 Hz, 1H), 2.94 (dt, J=44.8, 11.7 Hz, 1H), 2.83-2.69 (m, 5H), 2.65-2.46 (m, 1H), 1.93-1.51 (m, 4H), 1.24 (tt, J=7.3, 4.0 Hz, 3H). MS (ESI⁺) m/z 675 (M+H)⁺.

### Example 813

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-(pyrrolidin-2-ylmethyl)acetamide

The title compound was prepared as described in EXAMPLE 302, substituting tert-butyl 2-{[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]methyl}pyrrolidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆, 120° C.) δ ppm 8.29 (brs, 1H), 7.51-7.43 (m, 1H), 7.36-7.19 (m, 4H), 7.17-7.05 (m, 2H), 5.93-5.82 (m, 1H), 4.84-4.38 (m, 4H), 3.45-3.22 (m, 3H), 3.12 (dt, J=15.3, 7.4 Hz, 1H), 3.08-2.76 (m, 3H), 2.66 (d, J=4.9 Hz, 4H), 2.50-2.41 (m, 1H), 1.88-1.54 (m, 3H), 1.42-1.28 (m, 1H). MS (ESI⁺) m/z 524 (M+H)⁺.

### Example 814

### tert-butyl {4-[(4-fluorobenzyl)({5-[(methylcarbamoyl)amino]-2′,4′-dioxo-2,3-dihydro-3′H-spiro[indene-1,5′-[1,3]oxazolidin]-3′-yl}acetyl)amino]cyclohexylidene}acetate

### Example 814A

### tert-butyl 2-(4-((4-fluorobenzyl)amino)cyclohexylidene)acetate

The title compound was prepared as described in EXAMPLE 283A, substituting (4-fluorophenyl)methanamine for (S)-1-cyclopropylethanamine and tert-butyl 2-(4-oxocyclohexylidene)acetate for benzaldehyde.

### Example 814B

### tert-butyl {4-[(4-fluorobenzyl)({5-[(methylcarbamoyl)amino]-2′,4′-dioxo-2,3-dihydro-3′H-spiro[indene-1,5′-[1,3]oxazolidin]-3′-yl}acetyl)amino]cyclohexylidene}acetate

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and tert-butyl 2-(4-((4-fluorobenzyl)amino)cyclohexylidene)acetate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, CD3OD) δ 7.52 (s, 1H), 7.46-7.35 (m, 2H), 7.30-7.17 (m, 2H), 7.12 (t, J=8.7 Hz, 1H), 7.00 (t, J=8.8 Hz, 1H), 5.55 (d, J=12.5 Hz, 1H), 4.81-4.71 (m, 1H), 4.68-4.52 (m, 3H), 4.50-4.37 (m, 1H), 3.84 (t, J=14.1 Hz, 1H), 3.26-3.14 (m, 1H), 3.14-2.98 (m, 1H), 2.86-2.69 (m, 4H), 2.62-2.49 (m, 1H), 2.43-2.23 (m, 2H), 2.08-1.80 (m, 3H), 1.70-1.51 (m, 2H), 1.44 (d, J=4.3 Hz, 9H). MS (ESI⁺) m/z 635 (M+H)+.

### Example 816

### {4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclohexylidene}acetic acid

The title compound was prepared as described in EXAMPLE 302, substituting tert-butyl {4-[(4-fluorobenzyl)({5-[(methylcarbamoyl)amino]-2′,4′-dioxo-2,3-dihydro-3′H-spiro[indene-1,5′-[1,3]oxazolidin]-3′-yl}acetyl)amino]cyclohexylidene}acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, CD₃OD) δ 7.52 (s, 1H), 7.45-7.35 (m, 2H), 7.29-7.18 (m, 2H), 7.16-7.09 (m, 1H), 7.05-6.96 (m, 1H), 5.63 (d, J=12.2 Hz, 1H), 4.79-4.69 (m, 1H), 4.67-4.53 (m, 3H), 4.52-4.37 (m, 1H), 3.88 (t, J=13.4 Hz, 1H), 3.27-3.13 (m, 1H), 3.07 (dp, J=15.4, 5.0 Hz, 1H), 2.84-2.69 (m, 4H), 2.61-2.46 (m, 1H), 2.40-2.25 (m, 2H), 2.09-1.80 (m, 3H), 1.74-1.49 (m, 2H). MS (DCI) m/z 579 (M+H)⁺.

### Example 817

### 2-[(4S)-2,5-dioxo-5′-(1H-pyrazol-4-yl)-2′,3′-dihydro-H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 295, substituting 2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and tert-butyl 4-(4,4,5,5-tetramethyl-1,3,2-dioxaborolan-2-yl)-1H-pyrazole-1-carboxylate for 5-(4,4,5,5-tetramethyl-1,3,2-dioxaborolan-2-yl)pyridin-2-ol. ¹H NMR (400 MHz, DMSO-d₆) δ 12.93 (s, 1H), 8.81 (d, J=11.4 Hz, 1H), 8.19 (s, 1H), 7.92 (s, 1H), 7.67-6.85 (m, 7H), 5.45-4.95 (m, 1H), 4.94-4.07 (m, 4H), 3.05 (q, J=6.5 Hz, 2H), 2.57 (ddd, J=13.1, 9.3, 6.5 Hz, 1H), 2.33-2.12 (m, 1H), 1.33 (dd, J=24.0, 6.9 Hz, 3H). MS (ESI⁺) m/z 530 (M+H)⁺.

### Example 818

### N-(4-fluorobenzyl)-2-{5′-[(1-methyl-1H-pyrazol-3-yl)amino]-2,5-dioxo-2′,3′-dihydro-1H-spiro[imidazolidine-4,1′-inden]-1-yl}-N-[(2S)-1,1,1-trifluoropropan-2-yl]acetamide

The title compound was prepared as described in EXAMPLE 284D, substituting 2-(5′-bromo-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide and 1-methyl-1H-pyrazol-4-amine for diphenylmethanimine. ¹H NMR (400 MHz, dimethylsulfoxide-d₆) δ ppm 1.30 (dd, J=25.6, 6.9 Hz, 3H), 2.03-2.22 (m, 1H), 2.40-2.54 (m, 1H), 2.80-3.01 (m, 2H), 3.77 (d, J=2.1 Hz, 3H), 3.99-4.46 (m, 2H), 4.64-5.42 (m, 3H), 6.58-6.70 (m, 2H), 6.90-7.43 (m, 6H), 7.50-7.71 (m, 2H), 8.55-8.73 (m, 1H). MS (ESI⁺) m/z 559 (M+H)⁺.

### Example 819

### 2-[(4S)-5′-amino-2,5-dioxo-2′,3′-dihydro-H-spiro[imidazolidinee-4,1′-inden]-1-yl]-N-(4-fluorobenzyl)-N-[(2S)-1,1,1-trifluoropropan-2-yl]acetamide

To 2-[(4S)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide (200 mg, 0.384 mmol) in 1 mL tetrahydrofuran and 1 mL methanol was added 6M hydrogen chloride (640 μl, 3.84 mmol). The mixture was heated at 60° C. for 4 hours. The solution was cooled to room temperature, diluted with water and neutralized with saturated sodium bicarbonate solution. Extraction with ethyl acetate gave an organic layer that was dried with sodium sulfate, filtered and concentrated to give the title compound. ¹H NMR (400 MHz, DMSO-d₆) δ 8.14 (s, 1H), 7.32 (t, J=6.7 Hz, 2H), 7.10 (t, J=8.7 Hz, 2H), 6.87 (d, J=8.2 Hz, 1H), 6.65-6.27 (m, 2H), 5.16 (p, J=7.9 Hz, 1H), 4.93-4.11 (m, 4H), 2.89 (t, J=7.3 Hz, 2H), 2.50 (m, 1H), 2.15 (dt, J=13.9, 7.6 Hz, 1H), 1.33 (d, J=7.2 Hz, 3H). MS (ESI⁺) m/z 479 (M+H)⁺.

### Example 820

### 2-{4′-[(dimethylamino)methyl]-2,5-dioxo-2′,3′-dihydro-1H-spiro[imidazolidine-4, 1′-inden]-1-yl}-N-(4-fluorobenzyl)-N-[(2S)-1,1,1-trifluoropropan-2-yl]acetamide

A mixture of 2-(4′-bromo-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide (50 mg, 0.092 mmol), potassium ((dimethylamino)methyl)trifluoroborate (18.26 mg, 0.111 mmol), dicyclohexyl(2′,4′,6′-triisopropyl-[1,1′-biphenyl]-2-yl)phosphine (8.79 mg, 0.018 mmol), palladium(II) acetate (2.070 mg, 9.22 μmol) and cesium carbonate (90 mg, 0.3 mmol) in 2.5 ml dioxane and 0.5 ml water was purged with nitrogen and heated in a Biotage Initiator® microwave at 150° C. for 50 minutes. The mixture was concentrated and the residue was purified by reverse-phase HPLC to provide the title compound. ¹H NMR (500 MHz, DMSO-d₆) δ 9.70 (s, 1H), 8.94-8.83 (m, 1H), 7.48 (d, J=7.4 Hz, 1H), 7.39 (dt, J=8.9, 7.0 Hz, 3.3H), 7.27-7.17 (m, 2H), 7.11 (dd, J=18.5, 9.1 Hz, 0.7H), 5.41-5.30 (m, 0.7H), 5.07 (d, J=6.6 Hz, 0.3H), 4.94-4.67 (m, 2H), 4.45-4.25 (m, 3.3H), 4.08 (dd, J=17.0, 6.9 Hz, 0.7H), 3.20-3.05 (m, 2H), 2.79 (t, J=5.5 Hz, 6H), 2.61-2.54 (m, 1H), 2.24 (ddd, J=20.0, 13.2, 7.8 Hz, 1H), 1.39-1.26 (m, 3H). MS (ESI⁺) m/z 521 (M+H)⁺.

### Example 821

### N-(4-fluorobenzyl)-2-[4′-(morpholin-4-ylmethyl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 820, substituting potassium trifluoro(morpholinomethyl)borate for potassium ((dimethylamino)methyl)trifluoroborate. ¹H NMR (500 MHz, DMSO-d₆) δ 10.02 (s, 1H), 8.87 (dd, J=14.1, 3.0 Hz, 1H), 7.51 (d, J=7.2 Hz, 1H), 7.45-7.33 (m, 3.3H), 7.22 (td, J=8.7, 1.8 Hz, 2H), 7.11 (q, J=9.3 Hz, 0.7H), 5.44-5.27 (m, 0.7H), 5.10-5.05 (m, 0.3H), 4.94-4.66 (m, 2H), 4.45-4.30 (m, 3.3H), 4.13-4.05 (m, 0.7H) 3.96 (m, 2H), 3.38-3.07 (m, 6H), 2.57 (ddd, J=13.1, 7.8, 5.3 Hz, 1H), 2.24 (qd, J=13.2, 11.8, 7.6 Hz, 1H), 1.33 (ddd, J=31.8, 7.1, 2.7 Hz, 3H). MS (ESI⁺) 563.2 (M+H)⁺.

### Example 822

### 2-[2,5-dioxo-4′-(pyrrolidin-1-ylmethyl)-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 820, substituting potassium trifluoro(pyrrolidin-1-ylmethyl)borate for potassium ((dimethylamino)methyl)trifluoroborate. ¹H NMR (500 MHz, DMSO-d₆) δ 8.79 (d, J=15.4 Hz, 1H), 7.37 (dt, J=8.8, 4.8 Hz, 1.3H), 7.22 (dq, J=19.6, 7.6 Hz, 3H), 7.16-7.06 (m, 2H), 5.41-5.27 (m, 0.7H), 5.10-5.02 (m, 0.3H), 4.93-4.65 (m, 2H), 4.40 (dd, J=16.8, 5.0 Hz, 1.3H), 4.07 (d, J=17.0 Hz, 0.7H), 3.15-2.96 (m, 4H), 2.71 (dtd, J=20.6, 11.8, 3.1 Hz, 2H), 2.59-2.50 (m, 1H), 2.26-2.12 (m, 1H), 2.07-1.93 (m, 1H), 1.75-1.51 (m, 4H), 1.38-1.25 (m, 3H). MS (ESI⁺) 547 (M+H)⁺.

### Example 823

### 2-(4′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

The title compound was prepared as described in EXAMPLE 284D-E, substituting 2-(4′-bromo-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide. ¹H NMR (400 MHz, DMSO-d₆) δ 8.69 (d, J=9.1 Hz, 1H), 7.35 (d, J=5.6 Hz, 1.3H), 7.20 (t, J=8.8 Hz, 2H), 7.09 (dd, J=15.7, 8.7 Hz, 0.7H), 6.91 (t, J=7.6 Hz, 1H), 6.50 (d, J=7.8 Hz, 1H), 6.42 (dd, J=12.9, 7.2 Hz, 1H), 5.41-5.27 (m, 0.7H), 5.06 (m, 2.3H), 4.75 (ddd, J=31.8, 28.2, 13.4 Hz, 2H), 4.35 (ddd, J=26.0, 12.5, 7.3 Hz, 1.3H), 4.05 (dd, J=11.7, 4.9 Hz, 0.7H), 2.74 (dt, J=15.6, 7.7 Hz, 2H), 2.46-2.41 (m, 1H), 2.19-2.05 (m, 1H), 1.30 (dd, J=26.4, 6.8 Hz, 3H). MS (ESI⁺) m/z 479 (M+H)⁺.

### Example 824

### N-(4-fluorobenzyl)-2-[(1R)-5-(oxetan-3-ylamino)-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 283A, substituting 2-[(1R)-5-amino-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for (S)-1-cyclopropylethanamine and oxetan-3-one for benzaldehyde. ¹H NMR (400 MHz, DMSO-d₆, 120° C.) δ ppm 7.39-7.29 (m, 2H), 7.19-7.01 (m, 3H), 6.49-6.38 (m, 2H), 6.22 (brs, 1H), 5.27-5.09 (m, 1H), 4.89-4.77 (m, 3H), 4.68-4.48 (m, 3H), 4.48-4.30 (m, 3H), 3.13-2.87 (m, 2H), 2.67-2.57 (m, 1H), 2.45-2.37 (m, 1H), 1.37 (d, J=7.1 Hz, 3H). MS (ESI⁺) m/z 536 (M+H)⁺.

### Example 826

### N-[(1-acetylpyrrolidin-2-yl)methyl]-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 346, substituting N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-(pyrrolidin-2-ylmethyl)acetamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (400 MHz, DMSO-d₆, mixture of rotamers) δ ppm 8.71 (s, 1H), 7.60-7.48 (m, 1H), 7.40-7.09 (m, 6H), 6.09 (brs, 1H), 4.85-4.30 (m, 4H), 4.28-4.17 (m, 1H), 3.60-2.91 (m, 6H), 2.74-2.56 (m, 4H), 2.56-2.43 (m, 1H), 2.11-1.66 (m, 7H). MS (ESI⁺) m/z 566 (M+H)⁺.

### Example 827

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-{[1-(methylsulfonyl)pyrrolidin-2-yl]methyl}acetamide

The title compound was prepared as described in EXAMPLE 308, substituting N-(4-fluorobenzyl)-2-{(1R)-5-[(methylcarbamoyl)amino]-2′,4′-dioxo-2,3-dihydro-3′H-spiro[indene-,5′-[1,3]oxazolidin]-3′-yl}-N-(pyrrolidin-2-ylmethyl)acetamide for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide hydrochloride. ¹H NMR (400 MHz, DMSO-d₆, mixture of rotamers) δ ppm 8.69 (brs, 1H), 7.60-7.44 (m, 1H), 7.40-7.03 (m, 6H), 6.08 (brs, 1H), 4.88-4.34 (m, 4H), 4.15-3.90 (m, 1H), 3.71-3.50 (m, 1H), 3.34-2.85 (m, 8H), 2.74-2.57 (m, 4H), 2.54-2.42 (m, 1H), 2.05-1.66 (m, 4H). MS (ESI⁺) m/z 602 (M+H)⁺.

### Example 828

### 2-{[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]methyl}-N-methylpyrrolidine-1-carboxamide

The title compound was prepared as described in EXAMPLE 282, substituting N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-(pyrrolidin-2-ylmethyl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methylamine hydrochloride for 3,3-difluoroazetidine hydrochloride. ¹H NMR (400 MHz, DMSO-d₆, mixture of rotamers) δ ppm 8.77-8.61 (m, 1H), 7.60-7.49 (m, 1H), 7.39-7.09 (m, 6H), 6.09 (brs, 2H), 4.89-4.71 (m, 2H), 4.65-4.35 (m, 2H), 4.18-4.09 (m, 1H), 3.65-3.40 (m, 1H), 3.35-2.93 (m, 5H), 2.72-2.54 (m, 7H), 2.54-2.45 (m, 1H), 2.02-1.66 (m, 4H). MS (ESI⁺) m/z 581 (M+H)⁺.

### Example 829

### N-(3,4-difluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide

### Example 829A

### 2-((R)-5-bromo-2′,4′-dioxo-2,3-dihydrospiro[indene-1,5′-oxazolidin]-3′-yl)-N-(3,4-difluorobenzyl)-N—((S)-1,1,1-trifluoropropan-2-yl)acetamide

The title compound was prepared as described in EXAMPLE 281F, substituting (R)-5-bromo-2,3-dihydrospiro[indene-1,5′-oxazolidine]-2′,4′-dione for (R)-5-bromo-2,3-dihydrospiro[indene-1,5′-oxazolidine]-2′,4′-dione and (S)-2-bromo-N-(3,4-difluorobenzyl)-N-(1,1,1-trifluoropropan-2-yl)acetamide for (S)-2-bromo-N-(4-fluorobenzyl)-N-(1,1,1,1-trifluoropropan-2-yl)acetamide.

### Example 829B

### N-(3,4-difluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-((R)-5-bromo-2′,4′-dioxo-2,3-dihydrospiro[indene-1,5′-oxazolidin]-3′-yl)-N-(3,4-difluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.18 (d, J=5.5 Hz, 1H), 8.05-7.97 (m, 1H), 7.93 (d, J=5.3 Hz, 1H), 7.61 (d, J=1.5 Hz, 1H), 7.58-7.01 (m, 5H), 5.46-3.97 (m, 7H), 3.27-2.94 (m, 2H), 2.76-2.49 (m, 5H), 1.43-1.30 (m, 3H). MS (ESI⁺) m/z 620 (M+H)⁺.

### Example 830

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-{[1-(methylsulfamoyl)pyrrolidin-2-yl]methyl}acetamide

In a 4 mL vial, a solution of N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-(pyrrolidin-2-ylmethyl)acetamide (0.035 g, 0.067 mmol) and N-ethyl-N-isopropylpropan-2-amine (0.058 ml, 0.334 mmol) in N,N-dimethylformamide (0.669 ml) was treated with methylsulfamoyl chloride (0.019 g, 0.147 mmol), and the reaction mixture was stirred at room temperature for 16 hours. The reaction mixture was directly purified by reverse-phase HPLC to give the title compound. ¹H NMR (400 MHz, DMSO-d₆, mixture of rotamers) δ ppm 8.69 (s, 1H), 7.60-7.49 (m, 1H), 7.40-6.95 (m, 7H), 6.07 (brs, 1H), 4.85-4.35 (m, 4H), 4.08-3.90 (m, 1H), 3.65-3.50 (m, 1H), 3.31-2.92 (m, 5H), 2.71-2.57 (m, 4H), 2.57-2.45 (m, 4H), 2.03-1.64 (m, 4H). MS (ESI⁺⁾ m/z 617 (M+H)⁺.

### Example 832

### methyl trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylate

### Example 832A

### tert-butyl 2-(5′-bromo-2,5-dioxo-2′,3′-dihydrospiro[imidazolidine-4,1′-inden]-1-yl)acetate

5′-Bromo-2′,3′-dihydrospiro[imidazolidine-4,1′-indene]-2,5-dione was dissolved in N,N-dimethylformamide (50 ml). Tert-butyl 2-bromoacetate was added, followed by potassium carbonate. The reaction mixture was stirred at room temperature for 2 hours, poured into 150 ml water, and stirred vigorously. A solid precipitated and was collected by filtration with water and ether washes to give the title compound after vacuum drying.

### Example 832B

### 2-(5′-bromo-2,5-dioxo-2′,3′-dihydrospiro[imidazolidine-4,1′-inden]-1-yl)acetic acid

The title compound was prepared as described in EXAMPLE 302, substituting tert-butyl 2-(5′-bromo-2,5-dioxo-2′,3′-dihydrospiro[imidazolidine-4,1′-inden]-1-yl)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 832C

### trans-methyl 4-((4-fluorobenzyl)amino)cyclohexanecarboxylate

The title compound was prepared as described in EXAMPLE 283A, substituting trans-methyl 4-aminocyclohexanecarboxylate hydrochloride for (S)-1-cyclopropylethanamine and 4-fluorobenzaldehyde for benzaldehyde.

### Example 832D

The title compound was prepared as described in EXAMPLE 306, substituting 2-(5′-bromo-2,5-dioxo-2′,3′-dihydrospiro[imidazolidine-4,1′-inden]-1-yl)acetic acid for cyclopropanecarboxylic acid and trans-methyl 4-((4-fluorobenzyl)amino)cyclohexanecarboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (501 MHz, DMSO-d₆) δ 8.83 (d, J=13.4 Hz, 1H), 7.56 (d, J=1.8 Hz, 1H), 7.47 (ddd, J=11.3, 8.1, 1.8 Hz, 1H), 7.36 (dd, J=8.6, 5.5 Hz, 1H), 7.26-7.17 (m, 3H), 7.10 (t, J=8.8 Hz, 1H), 4.63 (s, 1H), 4.50 (s, 2H), 4.17 (s, 1H), 3.88-3.81 (m, 1H), 3.57 (d, J=4.9 Hz, 3H), 3.12-3.01 (m, 2H), 2.60-2.52 (m, 1H), 2.22 (tdd, J=15.3, 8.0, 5.1 Hz, 2H), 1.91-1.85 (m, 2H), 1.67-1.17 (m, 6H). MS (ESI⁺) m/z 586 (M+H)⁺.

### Example 833

### trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylic acid

The title compound was prepared as described in EXAMPLE 903, substituting methyl trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylate for methyl {3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]phenyl}acetate. ¹H NMR (400 MHz, DMSO-d₆) δ 8.82 (d, J=10.4 Hz, 1H), 7.56 (d, J=1.8 Hz, 1H), 7.52-7.42 (m, 1H), 7.36 (dd, J=8.6, 5.4 Hz, 1H), 7.28-7.14 (m, 3H), 7.10 (t, J=8.8 Hz, 1H), 4.50 (d, J=4.1 Hz, 2H), 4.17 (s, 1H), 3.83 (s, 1H), 3.04 (q, J=6.8 Hz, 2H), 2.62-2.50 (m, 2H), 2.27-2.16 (m, 1H), 2.14-2.02 (m, 1H), 1.88 (d, J=11.6 Hz, 2H), 1.67-1.21 (m, 6H). MS (ESI⁺) m/z 572 (M+H)⁺.

### Example 834

### trans-4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]cyclohexanecarboxylic acid

The title compound was prepared as described in EXAMPLE 295, substituting trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylic acid for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.76 (d, J=9.7 Hz, 1H), 8.13 (d, J=4.9 Hz, 1H), 7.86 (d, J=4.5 Hz, 1H), 7.56-7.34 (m, 3H), 7.30-7.16 (m, 3H), 7.10 (t, J=8.8 Hz, 1H), 4.63 (s, 1H), 4.50 (d, J=6.0 Hz, 2H), 4.18 (s, 5H), 3.04 (q, J=6.7 Hz, 2H), 2.63-2.53 (m, 1H), 2.31-2.03 (m, 2H), 1.89 (d, J=11.4 Hz, 2H), 1.67-1.25 (m, 7H). MS (ESI⁺) m/z 574 (M+H)⁺.

### Example 835

### 2-[(4R)-7′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

### Example 835A

### 7′-bromo-2′,3′-dihydrospiro[imidazolidine-4,1′-indene]-2,5-dione

The title compound was prepared as described in EXAMPLE 290B, substituting 7-bromo-2,3-dihydro-1H-inden-1-one for 6-bromobenzo[b]thiophen-3 (2H)-one 1,1-dioxide.

### Example 835B

### 2-[(4R)-7′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 281F, substituting 7′-bromo-2′,3′-dihydrospiro[imidazolidine-4,1′-indene]-2,5-dione for (R)-5-bromo-2,3-dihydrospiro[indene-1,5′-oxazolidine]-2′,4′-dione. Silica gel column chromatography provided both 2-[(4R)-7′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide and 2-[(4S)-7′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide, separately. ¹H NMR (400 MHz, DMSO-d₆, mixture of rotamers) δ ppm 8.86 (d, J=3.1 Hz, 1H), 7.51-7.05 (m, 7H), 5.43-5.02 (m, 1H), 4.97-4.66 (m, 2H), 4.50-4.07 (m, 2H), 3.20-2.95 (m, 2H), 2.62-2.52 (m, 1H), 2.34-2.18 (m, 1H), 1.41-1.23 (m, 3H). MS (ESI⁺) m/z 544 (M+H)⁺.

### Example 836

### trans-4-{(4-fluorobenzyl) [(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}cyclohexanecarboxylic acid

The title compound was prepared as described in EXAMPLE 295, substituting trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylic acid for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (501 MHz, CD₃OD) δ 8.03 (d, J=5.2 Hz, 1H), 7.89 (d, J=4.5 Hz, 1H), 7.53 (s, 1H), 7.48-7.44 (m, 1H), 7.43-7.32 (m, 2H), 7.24 (dd, J=8.6, 5.4 Hz, 1H), 7.11 (t, J=8.7 Hz, 1H), 7.04-6.96 (m, 1H), 4.67-4.53 (m, 3H), 4.38-4.24 (m, 2H), 3.87 (dt, J=12.0, 6.1 Hz, 1H), 3.21-3.06 (m, 2H), 2.80-2.69 (m, 4H), 2.42-2.31 (m, 1H), 2.30-2.11 (m, 1H), 2.07-1.94 (m, 2H), 1.86-1.66 (m, 2H), 1.65-1.40 (m, 5H). MS (ESI⁺) m/z 631 (M+H)⁺.

### Example 837

### 2-[(4S)-7′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 281F, substituting 7′-bromo-2′,3′-dihydrospiro[imidazolidine-4,1′-indene]-2,5-dione for (R)-5-bromo-2,3-dihydrospiro[indene-1,5′-oxazolidine]-2′,4′-dione. Silica gel column chromatography provided both 2-[(4R)-7′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide and 2-[(4S)-7′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide, separately. ¹H NMR (400 MHz, DMSO-d₆, mixture of rotamers) δ ppm 8.91-8.81 (m, 1H), 7.50-7.08 (m, 7H), 5.41-5.03 (m, 1H), 4.97-4.65 (m, 2H), 4.50-4.08 (m, 2H), 3.18-2.97 (m, 2H), 2.64-2.53 (m, 1H), 2.31-2.17 (m, 1H), 1.40-1.25 (m, 3H). MS (ESI⁺) m/z 542 (M+H)⁺.

### Example 838

### ethyl ({4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]piperidin-1-yl}sulfonyl)carbamate

### Example 838A

### tert-butyl 4-(2-(5′-bromo-2,5-dioxo-2′,3′-dihydrospiro[imidazolidine-4,1′-inden]-1-yl)-N-(4-fluorobenzyl)acetamido)piperidine-1-carboxylate

The title compound was prepared as described in EXAMPLE 306, substituting 2-(5′-bromo-2,5-dioxo-2′,3′-dihydrospiro[imidazolidine-4,1′-inden]-1-yl)acetic acid for cyclopropanecarboxylic acid and tert-butyl 4-((4-fluorobenzyl)amino)piperidine-1-carboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 838B

### 2-(5′-bromo-2,5-dioxo-2′,3′-dihydrospiro[imidazolidine-4,1′-inden]-1-yl)-N-(4-fluorobenzyl)-N-(piperidin-4-yl)acetamide

The title compound was prepared as described in EXAMPLE 302, substituting tert-butyl 4-(2-(5′-bromo-2,5-dioxo-2′,3′-dihydrospiro[imidazolidine-4,1′-inden]-1-yl)-N-(4-fluorobenzyl)acetamido)piperidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 838C

### ethyl (4-(2-(5′-bromo-2,5-dioxo-2′,3′-dihydrospiro[imidazolidine-4,1′-inden]-1-yl)-N-(4-fluorobenzyl)acetamido)piperidin-1-yl)sulfonylcarbamate

The title compound was prepared as described in EXAMPLE 678, substituting 2-(5′-bromo-2,5-dioxo-2′,3′-dihydrospiro[imidazolidine-4,1′-inden]-1-yl)-N-(4-fluorobenzyl)-N-(piperidin-4-yl)acetamide for N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide.

### Example 838D

### ethyl ({4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]piperidin-1-yl}sulfonyl)carbamate

The title compound was prepared as described in EXAMPLE 295, substituting ethyl (4-(2-(5′-bromo-2,5-dioxo-2′,3′-dihydrospiro[imidazolidine-4,1′-inden]-1-yl)-N-(4-fluorobenzyl)acetamido)piperidin-1-yl)sulfonylcarbamate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, methanol-d₄) δ 7.94 (d, J=3.3 Hz, 1H), 7.81 (d, J=2.3 Hz, 1H), 7.49 (s, 1H), 7.46-7.32 (m, 3H), 7.23 (dd, J=8.4, 5.3 Hz, 1H), 7.11 (t, J=8.5 Hz, 1H), 6.99 (t, J=8.6 Hz, 1H), 4.68-4.55 (m, 3H), 4.35 (d, J=2.3 Hz, 1H), 4.15 (qd, J=7.1, 3.4 Hz, 2H), 3.97 (s, 1H), 3.90 (d, J=2.0 Hz, 3H), 3.81 (t, J=11.4 Hz, 2H), 3.13 (d, J=7.4 Hz, 2H), 3.06-2.84 (m, 1H), 2.71 (ddd, J=13.2, 7.7, 4.8 Hz, 1H), 2.33 (dd, J=13.8, 7.4 Hz, 1H), 1.73 (d, J=36.9 Hz, 5H), 1.26 (td, J=7.2, 2.8 Hz, 3H). MS (ESI⁺) m/z 682 (M+H)⁺.

### Example 839

### ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]piperidin-1-yl}sulfonyl)carbamate

The title compound was prepared as described in EXAMPLE 678, substituting N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-piperidin-3-yl]acetamide for N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide. ¹H NMR (501 MHz, methanol-d₄) δ 7.52 (s, 1H), 7.46-7.35 (m, 2H), 7.29-7.17 (m, 2H), 7.14 (t, J=8.6 Hz, 1H), 7.07-6.97 (m, 1H), 4.67 (dt, J=21.9, 5.1 Hz, 3H), 4.58-4.41 (m, 1H), 4.27-4.02 (m, 3H), 3.78-3.64 (m, 2H), 3.24-3.13 (m, 1H), 3.06 (ddt, J=16.4, 8.6, 4.2 Hz, 1H), 2.94 (dt, J=44.8, 11.7 Hz, 1H), 2.79 (d, J=14.3 Hz, 1H), 2.76 (d, J=3.1 Hz, 3H), 2.75-2.68 (m, 1H), 2.63-2.48 (m, 1H), 1.83-1.55 (m, 4H), 1.24 (dt, J=7.0, 3.3 Hz, 3H). MS (ESI⁺) m/z 675 (M+H)⁺.

### Example 840

### {4-[(4-fluorobenzyl)({5-[(methylcarbamoyl)amino]-2′,4′-dioxo-2,3-dihydro-3′H-spiro[indene-1,5′-[1,3]oxazolidin]-3′-yl}acetyl)amino]cyclohexyl}acetic acid

The title compound was prepared as described in EXAMPLE 894, substituting {4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclohexylidene}acetic acid for 2-[2,5-dioxo-4′-(1,2,3,6-tetrahydropyridin-4-yl)-2′,3′-dihydro-H-spiro[imidazolidine-4,1′-inden]-1-yl]-N-(4-fluorobenzyl)-N-[(2S)-1,1,1-trifluoropropan-2-yl]acetamide. ¹H NMR (501 MHz, CD₃OD) δ 7.52 (s, 1H), 7.38 (dd, J=13.1, 8.2 Hz, 2H), 7.31-7.19 (m, 2H), 7.21-7.07 (m, 2H), 7.05-6.95 (m, 1H), 4.71-4.64 (m, 2H), 4.49-4.24 (m, 2H), 3.24-3.14 (m, 1H), 2.83-2.70 (m, 4H), 2.60-2.48 (m, 1H), 2.40-2.31 (m, 1H), 2.25-2.13 (m, 2H), 1.88-1.74 (m, 2H), 1.73-1.47 (m, 6H), 1.26-1.07 (m, 1H). MS (ESI⁺) m/z 581 (M+H)⁺.

### Example 841

### 2-[(4S)-5′-{[(2-cyanoethyl)carbamoyl]amino}-2,5-dioxo-2′,3′-dihydro-1H-spiro[imidazolidine-4, 1′-inden]-1-yl]-N-(4-fluorobenzyl)-N-[(2S)-1,1,1-trifluoropropan-2-yl]acetamide

The title compound was prepared as described in EXAMPLE 282, substituting 2-[(4S)-5′-amino-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and 3-aminopropanenitrile for 3,3-difluoroazetidine hydrochloride. ¹H NMR (400 MHz, DMSO-d₆) δ 8.29 (d, J=12.2 Hz, 2H), 7.39 (d, J=1.8 Hz, 1H), 7.33 (dd, J=8.5, 5.4 Hz, 2H), 7.19-7.03 (m, 4H), 6.27 (s, 1H), 5.25 (m, 1H), 4.84-4.06 (m, 4H), 3.36 (q, J=6.1 Hz, 2H), 2.98 (t, J=7.2 Hz, 2H), 2.64 (t, J=6.5 Hz, 2H), 2.54 (dt, J=13.4, 6.7 Hz, 1H), 2.21 (dt, J=13.2, 7.6 Hz, 1H), 1.33 (d, J=7.0 Hz, 3H). MS (ESI⁺) m/z 575 (M+H)⁺.

### Example 842

### N-(4-fluorobenzyl)-2-{(4S)-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide

The title compound was prepared as described in EXAMPLE 282, substituting 2-[(4S)-5′-amino-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methanamine for 3,3-difluoroazetidine hydrochloride. ¹H NMR (400 MHz, DMSO-d₆) δ 8.10 (s, 1H), 7.95 (s, 1H), 7.22 (s, 1H), 7.17 (dd, J=8.4, 5.4 Hz, 2H), 7.03-6.84 (m, 5H), 5.62 (q, J=4.7 Hz, 1H), 5.00 (p, J=7.8 Hz, 1H), 4.75-3.72 (m, 4H), 2.82 (t, J=7.2 Hz, 2H), 2.50 (d, J=4.6 Hz, 3H), 2.38 (dt, J=13.3, 6.6 Hz, 1H), 2.04 (dt, J=13.6, 7.6 Hz, 1H), 1.18 (d, J=7.0 Hz, 3H). MS (ESI⁺) m/z 536 (M+H)⁺.

### Example 843

### N-(4-fluorobenzyl)-2-{(4S)-5′-[(methylcarbamoyl)amino]-2,3′,5-trioxo-2′,3′-dihydro-1H-spiro[imidazolidine-4,1′-inden]-1-yl}-N-[(2S)-1,1,1-trifluoropropan-2-yl]acetamide

The title compound was prepared as described in EXAMPLE 770B, substituting 2-[(4S)-5′-bromo-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(7-bromo-2′,4′-dioxospiro[isochroman-4,5′-oxazolidin]-3′-yl)-N-(4-fluorobenzyl)-N—((S)-1,1,1-trifluoropropan-2-yl)acetamide. ¹H NMR (400 MHz, DMSO-d₆) δ 8.30 (d, J=26.8 Hz, 2H), 7.64 (d, J=2.0 Hz, 1H), 7.48 (dd, J=8.5, 2.1 Hz, 1H), 7.29 (d, J=8.2 Hz, 1H), 7.14 (dd, J=8.5, 5.4 Hz, 2H), 6.92 (t, J=8.7 Hz, 2H), 5.73 (d, J=5.3 Hz, 1H), 4.98 (dd, J=11.6, 4.9 Hz, 1H), 4.70-3.95 (m, 4H), 2.84 (d, J=18.4 Hz, 1H), 2.70 (s, 1H), 2.50 (d, J=4.6 Hz, 3H), 1.16 (d, J=7.0 Hz, 3H). MS (ESI⁺) m/z 550 (M+H)⁺.

### Example 844

### N-(4-fluorobenzyl)-2-{(3′S,4S)-3′-hydroxy-5′-[(methylcarbamoyl)amino]-2,5-dioxo-2′,3′-dihydro-1H-spiro[imidazolidine-4,1′-inden]-1-yl}-N-[(2S)-1, 1,1-trifluoropropan-2-yl]acetamide

The title compound was prepared as described in EXAMPLE 570A, substituting N-(4-fluorobenzyl)-2-{(4S)-5′-[(methylcarbamoyl)amino]-2,3′,5-trioxo-2′,3′-dihydro-1H-spiro[imidazolidine-4,1′-inden]-1-yl}-N-[(2S)-1,1,1-trifluoropropan-2-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide. ¹H NMR (400 MHz, DMSO-d₆) δ 8.21 (d, J=30.9 Hz, 2H), 7.50 (d, J=1.9 Hz, 1H), 7.34 (dd, J=8.4, 5.4 Hz, 2H), 7.24 (dd, J=8.2, 2.0 Hz, 1H), 7.10 (q, J=8.5 Hz, 3H), 5.78 (d, J=5.1 Hz, 1H), 5.21 (dq, J=21.1, 8.5, 7.6 Hz, 2H), 4.88-4.19 (m, 5H), 2.66 (d, J=4.6 Hz, 3H), 2.47-2.21 (m, 2H), 1.34 (d, J=7.0 Hz, 3H). MS (ESI⁺) m/z 550 (M−H)⁺.

### Example 845

### N-(4-fluorobenzyl)-2-{(3′R,4S)-3′-fluoro-5′-[1-(2-hydroxyethyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide

The title compound was prepared as described in EXAMPLE 295, substituting 2-[(3′R,4S)-5′-bromo-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)ethanol for 5-(4,4,5,5-tetramethyl-1,3,2-dioxaborolan-2-yl)pyridin-2-ol. ¹H NMR (400 MHz, DMSO-d₆) δ 8.61 (s, 1H), 8.10 (s, 1H), 7.84 (s, 1H), 7.73-7.56 (m, 2H), 7.34 (t, J=7.1 Hz, 3H), 7.12 (t, J=8.7 Hz, 2H), 6.13 (ddd, J=56.9, 6.7, 4.7 Hz, 1H), 5.33-4.96 (m, 1H), 4.89-4.23 (m, 4H), 4.19 (t, J=5.8 Hz, 2H), 3.83 (t, J=5.9 Hz, 2H), 3.06 (td, J=13.5, 6.9 Hz, 1H), 2.43-2.27 (m, 1H), 1.35 (d, J=7.0 Hz, 3H). MS (ESI⁺) m/z 592 (M+H)⁺.

### Example 846

### ethyl ({3-[(4-fluorobenzyl)({(1S)-5-[(methylcarbamoyl)amino]-2′,4′-dioxo-2,3-dihydro-3′H-spiro[indene-1,5′-[1,3]oxazolidin]-3′-yl}acetyl)amino]-3-methylazetidin-1-yl}sulfonyl)carbamate

### Example 846A

### tert-butyl 3-((4-fluorobenzyl)amino)-3-methylazetidine-1-carboxylate

The title compound was prepared as described in EXAMPLE 283A, substituting tert-butyl 3-amino-3-methylazetidine-1-carboxylate for (S)-1-cyclopropylethanamine and 4-fluorobenzaldehyde for benzaldehyde.

### Example 846B

### tert-butyl 3-(2-bromo-N-(4-fluorobenzyl)acetamido)-3-methylazetidine-1-carboxylate

The title compound was prepared as described in EXAMPLE 281B, substituting tert-butyl 3-((4-fluorobenzyl)amino)-3-methylazetidine-1-carboxylate for (S)-1,1,1-trifluoro-N-(4-fluorobenzyl)propan-2-amine.

### Example 846C

### (R)-tert-butyl 3-(2-(5-bromo-2′,4′-dioxo-2,3-dihydrospiro[indene-1,5′-oxazolidin]-3′-yl)-N-(4-fluorobenzyl)acetamido)-3-methylazetidine-1-carboxylate

The title compound was prepared as described in EXAMPLE 281F, substituting (R)-5-bromo-2,3-dihydrospiro[indene-1,5′-oxazolidine]-2′,4′-dione for (R)-5-bromo-2,3-dihydrospiro[indene-1,5′-oxazolidine]-2′,4′-dione and tert-butyl 3-(2-bromo-N-(4-fluorobenzyl)acetamido)-3-methylazetidine-1-carboxylate for (S)-2-bromo-N-(4-fluorobenzyl)-N-(1,1,1-trifluoropropan-2-yl)acetamide.

### Example 846D

### (R)-tert-butyl 3-(N-(4-fluorobenzyl)-2-(5-(3-methylureido)-2′,4′-dioxo-2,3-dihydrospiro[indene-1,5′-oxazolidin]-3′-yl)acetamido)-3-methylazetidine-1-carboxylate

The title compound was prepared as described in EXAMPLE 770B, substituting (R)-tert-butyl 3-(2-(5-bromo-2′,4′-dioxo-2,3-dihydrospiro[indene-1,5′-oxazolidin]-3′-yl)-N-(4-fluorobenzyl)acetamido)-3-methylazetidine-1-carboxylate for 2-(7-bromo-2′,4′-dioxospiro[isochroman-4,5′-oxazolidin]-3′-yl)-N-(4-fluorobenzyl)-N—((S)-1,1,1-trifluoropropan-2-yl)acetamide.

### Example 846E

### (R)—N-(4-fluorobenzyl)-N-(3-methylazetidin-3-yl)-2-(5-(3-methylureido)-2′,4′-dioxo-2,3-dihydrospiro[indene-1,5′-oxazolidin]-3′-yl)acetamide

The title compound was prepared as described in EXAMPLE 302, substituting (R)-tert-butyl 3-(N-(4-fluorobenzyl)-2-(5-(3-methylureido)-2′,4′-dioxo-2,3-dihydrospiro[indene-1,5′-oxazolidin]-3′-yl)acetamido)-3-methylazetidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 846F

### ethyl ({3-[(4-fluorobenzyl)({(1S)-5-[(methylcarbamoyl)amino]-2′,4′-dioxo-2,3-dihydro-3′H-spiro[indene-1,5′-[1,3]oxazolidin]-3′-yl}acetyl)amino]-3-methylazetidin-1-yl}sulfonyl)carbamate

The title compound was prepared as described in EXAMPLE 678, substituting (R)—N-(4-fluorobenzyl)-N-(3-methylazetidin-3-yl)-2-(5-(3-methylureido)-2′,4′-dioxo-2,3-dihydrospiro[indene-1,5′-oxazolidin]-3′-yl)acetamide for N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide. ¹H NMR (400 MHz, dimethylsulfoxide-d₆) δ ppm 1.16 (t, J=7.1 Hz, 3H), 1.50 (s, 3H), 2.36-2.47 (m, 1H), 2.52-2.67 (m, 4H), 2.88-3.16 (m, 2H), 3.69 (d, J=8.2 Hz, 2H), 3.94-4.30 (m, 6H), 4.62 (s, 2H), 6.08 (s, 1H), 7.14-7.63 (m, 7H), 8.68 (s, 1H), 11.32 (s, 1H). MS (ESI⁺) m/z 661 (M+H)⁺.

### Example 847

### 2-[4′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 346, substituting 2-(4′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (400 MHz, DMSO-d₆) δ 9.43 (s, 1H), 8.82 (d, J=9.9 Hz, 1H), 7.61 (d, J=8.0 Hz, 1H), 7.36 (s, 1.3H), 7.21 (d, J=8.6 Hz, 3H), 7.09 (dd, J=16.0, 8.7 Hz, 0.7H), 7.00 (dd, J=12.0, 7.7 Hz, 1H), 5.41-5.26 (m, 0.7H), 5.05 (s, 0.3H), 4.78 (ddd, J=41.0, 29.4, 18.7 Hz, 2H), 4.35 (dd, J=22.9, 12.0 Hz, 1.3H), 4.10-4.01 (m, 0.7H), 2.96 (d, J=6.2 Hz, 2H), 2.58-2.48 (m, 1H), 2.19 (dd, J=18.5, 7.3 Hz, 1H), 2.06 (s, 3H), 1.30 (dd, J=26.2, 6.9 Hz, 3H). MS (ESI⁺) m/z 521 (M+H)⁺.

### Example 848

### 2-[(4R)-5′-bromo-6′-fluoro-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

### Example 848A

### (R)-5′-bromo-6′-fluorospiro[imidazolidine-4,1′-indene]-2,3′,5(2′H)-trione

The mixture of diastereomers of 5′-bromo-6′-fluorospiro[imidazolidine-4,1′-indene]-2,3′,5(2′H)-trione was separated by chiral prep-SFC (Dacel column) to give (R)-5′-bromo-6′-fluorospiro[imidazolidine-4,1′-indene]-2,3′,5(2′H)-trione (eluted first) and (S)-5′-bromo-6′-fluorospiro[imidazolidine-4,1′-indene]-2,3′,5(2′H)-trione (eluted second).

### Example 848B

### 2-[(4R)-5′-bromo-6′-fluoro-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 281F, substituting (R)-5′-bromo-6′-fluorospiro[imidazolidine-4,1′-indene]-2,3′,5(2′H)-trione for (R)-5-bromo-2,3-dihydrospiro[indene-1,5′-oxazolidine]-2′,4′-dione. ¹H NMR (500 MHz, DMSO-d₆) δ 8.98 (d, J=10.7 Hz, 1H), 8.14 (d, J=6.3 Hz, 1H), 7.69 (dd, J=10.8, 7.9 Hz, 1H), 7.36 (dd, J=8.6, 5.3 Hz, 1.3H), 7.27-7.17 (m, 2H), 7.12 (t, J=8.8 Hz, 0.7H), 5.43-5.32 (m, 0.7H), 5.10-5.05 (m, 0.3H), 4.96-4.66 (m, 2H), 4.54-4.33 (m, 1.3H), 4.09 (d, J=17.1 Hz, 0.7H), 3.16 (dd, J=18.7, 14.0 Hz, 1H), 3.01 (dd, J=20.8, 18.8 Hz, 1H), 1.32 (dd, J=30.1, 7.0 Hz, 3H). MS (ESI⁺) m/z 574 (M+H)⁺.

### Example 850

### N-(4-fluorobenzyl)-2-{(3′R,4S)-3′-fluoro-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide

The title compound was prepared as described in EXAMPLE 514, substituting N-(4-fluorobenzyl)-2-{(3′S,4S)-3′-hydroxy-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide. ¹H NMR (400 MHz, DMSO-d₆) δ 8.49 (s, 1H), 8.33 (s, 1H), 7.64 (s, 1H), 7.47-6.99 (m, 6H), 6.05 (ddd, J=56.9, 6.7, 4.7 Hz, 1H), 5.85 (s, 1H), 5.14 (m, 1H), 4.85-4.11 (m, 4H), 2.99 (ddd, J=14.3, 12.5, 6.8 Hz, 1H), 2.66 (d, J=2.4 Hz, 3H), 2.33 (ddd, J=25.5, 14.3, 4.6 Hz, 1H), 1.32 (d, J=7.1 Hz, 3H). MS (ESI⁺) m/z 554 (M+H)⁺.

### Example 851

### N-(3,4-difluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(2S)-1,1,1-trifluoropropan-2-yl]acetamide

The title compound was prepared as described in EXAMPLE 770B, substituting 2-((R)-5-bromo-2′,4′-dioxo-2,3-dihydrospiro[indene-1,5′-oxazolidin]-3′-yl)-N-(3,4-difluorobenzyl)-N—((S)-1,1,1-trifluoropropan-2-yl)acetamide for 2-(7-bromo-2′,4′-dioxospiro[isochroman-4,5′-oxazolidin]-3′-yl)-N-(4-fluorobenzyl)-N—((S)-1,1,1-trifluoropropan-2-yl)acetamide. ¹H NMR (400 MHz, DMSO-d₆) δ 8.69 (d, J=4.2 Hz, 1H), 7.63-6.99 (m, 6H), 6.07 (d, J=5.4 Hz, 1H), 5.49-4.13 (m, 5H), 3.18-2.88 (m, 2H), 2.71-2.36 (m, 5H), 1.40-1.28 (m, 3H). MS (ESI⁺) m/z 555 (M+H)⁺.

### Example 852

### N-(4-fluorobenzyl)-2-{7′-[(methylcarbamoyl)amino]-2,2′,5-trioxo-2′,3′-dihydro-1H, 1′H-spiro[imidazolidine-4,4′-quinolin]-1-yl}-N-[(2S)-1,1,1-trifluoropropan-2-yl]acetamide

The title compound was prepared as described in EXAMPLE 770B, substituting 2-(7′-bromo-2,2′,5-trioxo-2′,3′-dihydro-1H,1′H-spiro[imidazolidine-4,4′-quinolin]-1-yl)-N-(4-fluorobenzyl)-N-[(2S)-1,1,1-trifluoropropan-2-yl]acetamide for 2-(7-bromo-2′,4′-dioxospiro[isochroman-4,5′-oxazolidin]-3′-yl)-N-(4-fluorobenzyl)-N—((S)-1,1,1-trifluoropropan-2-yl)acetamide. ¹H NMR (400 MHz, DMSO-d₆) δ 11.72 (s, 1H), 8.88 (m, 1H), 7.75-6.01 (m, 9H), 5.42-3.11 (m, 7H), 2.68-2.60 (m, 3H), 1.38-1.24 (m, 3H). MS (ESI⁺) m/z 566 (M+H)⁺.

### Example 853

### tert-butyl (4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetate

The title compound was prepared as described in EXAMPLE 306, substituting 2-(5′-bromo-2,5-dioxo-2′,3′-dihydrospiro[imidazolidine-4,1′-inden]-1-yl)acetic acid for cyclopropanecarboxylic acid and tert-butyl 2-(4-((4-fluorobenzyl)amino)cyclohexylidene)acetate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆) δ 8.80 (d, J=11.4 Hz, 1H), 7.53 (d, J=1.7 Hz, 1H), 7.52-7.40 (m, 1H), 7.33 (dd, J=8.5, 5.4 Hz, 1H), 7.25-7.13 (m, 3H), 7.06 (q, J=8.8, 8.0 Hz, 1H), 5.49 (d, J=7.2 Hz, 1H), 4.65-4.04 (m, 5H), 3.71-3.62 (m, 1H), 3.02 (q, J=6.9 Hz, 2H), 2.60-2.50 (m, 1H), 2.30-2.13 (m, 3H), 1.98-1.42 (m, 5H), 1.36 (d, J=3.0 Hz, 9H). MS (ESI⁺) m/z 640, 642 (M+H)⁺.

### Example 854

### (4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetic acid

The title compound was prepared as described in EXAMPLE 302, substituting tert-butyl (4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, CD₃OD) δ 7.41 (d, J=3.1 Hz, 1H), 7.39-7.17 (m, 4H), 7.06 (t, J=8.6 Hz, 1H), 6.96 (t, J=8.6 Hz, 1H), 4.54 (d, J=5.1 Hz, 2H), 4.33-3.62 (m, 4H), 3.09-2.93 (m, 3H), 2.41-2.26 (m, 1H), 2.25-2.10 (m, 1H), 2.03-1.90 (m, 2H), 1.79-1.65 (m, 2H), 1.59-1.37 (m, 4H). MS (ESI⁺) m/z 584, 586 (M+H)⁺.

### Example 855

### {4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid

The title compound was prepared as described in EXAMPLE 295, substituting (4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetic acid for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, CD₃OD) δ 7.93 (d, J=3.4 Hz, 1H), 7.79 (d, J=2.5 Hz, 1H), 7.48 (s, 1H), 7.47-7.31 (m, 3H), 7.21 (dd, J=8.5, 5.4 Hz, 1H), 7.09 (t, J=8.7 Hz, 1H), 6.96 (t, J=8.7 Hz, 1H), 5.60 (d, J=11.8 Hz, 1H), 4.69-4.48 (m, 3H), 4.40-4.26 (m, 1H), 4.19-4.08 (m, 1H), 3.89 (s, 3H), 3.88-3.78 (m, 1H), 3.19-3.04 (m, 2H), 2.79-2.66 (m, 1H), 2.43-2.23 (m, 3H), 2.06-1.74 (m, 3H), 1.67-1.45 (m, 2H). MS (ESI⁺) m/z 586 (M+H)⁺.

### Example 856

### (4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}cyclohexylidene)acetic acid

The title compound was prepared as described in EXAMPLE 295, substituting (4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetic acid for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, CD₃OD) δ 8.02 (d, J=3.4 Hz, 1H), 7.87 (d, J=2.6 Hz, 1H), 7.51 (s, 1H), 7.49-7.42 (m, 1H), 7.42-7.32 (m, 2H), 7.21 (dd, J=8.5, 5.3 Hz, 1H), 7.09 (t, J=8.7 Hz, 1H), 6.97 (t, J=8.8 Hz, 1H), 5.60 (d, J=11.8 Hz, 1H), 4.69-4.47 (m, 3H), 4.33 (t, J=2.0 Hz, 1H), 4.15 (m, 1H), 3.85 (t, J=13.0 Hz, 1H), 3.22-3.06 (m, 2H), 2.80-2.65 (m, 4H), 2.42-2.25 (m, 3H), 2.01 (s, 3H), 1.70-1.47 (m, 2H). MS (ESI⁺) m/z 643 (M+H)⁺.

### Example 857

### tert-butyl {3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclobutyl}carbamate

### Example 857A

### tert-butyl (3-((4-fluorobenzyl)amino)cyclobutyl)carbamate

The title compound was prepared as described in EXAMPLE 283A, substituting tert-butyl (3-aminocyclobutyl)carbamate for (S)-1-cyclopropylethanamine and 4-fluorobenzaldehyde for benzaldehyde.

### Example 857B

### tert-butyl {3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclobutyl}carbamate

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and tert-butyl (3-((4-fluorobenzyl)amino)cyclobutyl)carbamate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆) δ 8.68 (d, J=2.6 Hz, 1H), 7.55 (d, J=3.8 Hz, 1H), 7.42-7.04 (m, 7H), 6.27-5.90 (m, 1H), 5.20-4.14 (m, 5H), 3.77 (s, 1H), 3.26-2.89 (m, 2H), 2.70-2.60 (m, 4H), 2.49-2.28 (m, 2H), 2.05 (d, J=49.4 Hz, 2H), 1.37 (d, J=5.2 Hz, 9H). LC-MS (APCI): 610 (M+H)⁺.

### Example 858

### N-(3-aminocyclobutyl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 302, substituting tert-butyl {3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclobutyl}carbamate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 9.11-8.76 (m, 1H), 8.46-8.03 (m, 3H), 7.60-7.38 (m, 1H), 7.41-6.93 (m, 6H), 6.24 (d, J=18.7 Hz, 1H), 5.37-4.07 (m, 4H), 3.21-2.77 (m, 2H), 2.60 (d, J=2.6 Hz, 4H), 2.40-2.02 (m, 1H). LC-MS (APCI): 510 (M+H)⁺.

### Example 859

### N-[3-(acetylamino)cyclobutyl]-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 346, substituting N-(3-aminocyclobutyl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (400 MHz, DMSO-d₆) δ 8.66 (d, J=3.1 Hz, 1H), 8.21 (dd, J=35.4, 6.4 Hz, 1H), 7.59-7.43 (m, 1H), 7.38-6.92 (m, 6H), 6.05 (t, J=4.7 Hz, 1H), 5.20-3.76 (m, 5H), 3.18-2.89 (m, 2H), 2.60 (t, J=3.8 Hz, 4H), 2.53-2.28 (m, 2H), 2.17-1.88 (m, 1H), 1.84-1.62 (m, 3H). LC-MS (APCI): 552 (M+H)⁺.

### Example 860

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-{3-[(methylsulfonyl)amino]cyclobutyl}acetamide

The title compound was prepared as described in EXAMPLE 308, substituting N-(3-aminocyclobutyl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide hydrochloride. ¹H NMR (400 MHz, DMSO-d₆) δ 8.66 (d, J=2.7 Hz, 1H), 7.51 (d, J=3.4 Hz, 1H), 7.44 (dd, J=7.1, 2.6 Hz, 1H), 7.34-7.01 (m, 5H), 6.04 (t, J=4.3 Hz, 1H), 5.04-4.08 (m, 5H), 3.71 (s, 1H), 3.16-2.89 (m, 2H), 2.89-2.74 (m, 4H), 2.60 (dd, J=4.7, 2.6 Hz, 3H), 2.53-2.36 (m, J=3.7 Hz, 3H), 2.33-2.05 (m, 2H). LC-MS (ESI): 588 (M+H)⁺.

### Example 861

### N-(4-fluorobenzyl)-2-[(3′R,4S)-3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

The title compound was prepared as described in EXAMPLE 295, substituting 2-[(3′R,4S)-5′-bromo-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.33 (d, J=2.7 Hz, 1H), 7.96 (s, 1H), 7.72 (s, 1H), 7.55 (d, J=1.5 Hz, 1H), 7.46 (dd, J=7.9, 1.6 Hz, 1H), 7.31 (dd, J=8.5, 5.4 Hz, 2H), 7.21-7.02 (m, 3H), 5.18 (dt, J=32.8, 7.5 Hz, 3H), 4.86-4.09 (m, 4H), 3.85 (s, 3H), 2.9 (m, 1H), 2.10 (dd, J=13.2, 6.6 Hz, 1H), 1.32 (d, J=7.1 Hz, 3H). MS (ESI⁺) m/z 560 (M+H)⁺.

### Example 862

### 2-[(4R)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 281F, substituting (R)—N-(2,5-dioxo-2′,3′-dihydrospiro[imidazolidine-4,1′-inden]-5′-yl)acetamide for (R)-5-bromo-2,3-dihydrospiro[indene-1,5′-oxazolidine]-2′,4′-dione. ¹H NMR (400 MHz, DMSO-d₆) δ 9.49 (s, 1H), 8.30 (s, 1H), 7.69-7.47 (m, 1H), 7.40-7.19 (m, 3H), 7.21-6.85 (m, 3H), 5.25 (m, 1H), 4.92-4.07 (m, 4H), 2.99 (t, J=7.2 Hz, 2H),), 2.61-2.49 (m, 1H), 2.31-2.13 (m, 1H), 2.01 (s, 3H), 1.32 (d, J=7.0 Hz, 3H). MS (ESI⁺) m/z 521 (M+H)⁺.

### Example 863

### N-(4-fluorobenzyl)-2-[(3′E,4S)-3′-(hydroxyimino)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide

A mixture of N-(4-fluorobenzyl)-2-[(4S)-5′-(1-methyl-1H-pyrazol-4-yl)-2,3′,5-trioxo-2′,3′-dihydro-1H-spiro[imidazolidine-4,1′-inden]-1-yl]-N-[(2S)-1,1,1-trifluoropropan-2-yl]acetamide (40 mg, 0.072 mmol), hydroxylamine hydrochloride (5.98 mg, 0.086 mmol) and sodium acetate (11.77 mg, 0.143 mmol) in 2 mL methanol was stirred at room temperature overnight. The mixture was diluted with water (15 mL) and extracted with ethyl acetate (2×50 mL). The organic layers were washed with brine, dried over sodium sulfate, filtered and concentrated to give the title compound. ¹H NMR (400 MHz, DMSO-d₆) δ 10.80 (s, 1H), 8.61-8.34 (m, 1H), 8.05 (s, 1H), 7.84-6.99 (m, 7H), 5.18 (m, 1H), 4.88-4.14 (m, 4H), 3.85 (s, 3H), 3.33 (d, J=18.4 Hz, 1H), 2.96 (dd, J=17.6, 7.4 Hz, 1H), 1.33 (d, J=7.1 Hz, 3H). MS (ESI⁺) m/z 573 (M+H)⁺.

### Example 864

### 2,2′-({3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclobutyl}imino)diacetic acid

### Example 864A

### (R)-di-tert-butyl 2,2′-((3-(N-(4-fluorobenzyl)-2-(5-(3-methylureido)-2′,4′-dioxo-2,3-dihydrospiro[indene-1,5′-oxazolidin]-3′-yl)acetamido)cyclobutyl)azanediyl)diacetate

The title compound was prepared as described in EXAMPLE 832A, substituting N-(3-aminocyclobutyl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide for 5′-bromo-2′,3′-dihydrospiro[imidazolidine-4,1′-indene]-2,5-dione.

### Example 864B

### 2,2′-({3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclobutyl}imino)diacetic acid

The title compound was prepared as described in EXAMPLE 302, substituting (R)-di-tert-butyl 2,2′-((3-(N-(4-fluorobenzyl)-2-(5-(3-methylureido)-2′,4′-dioxo-2,3-dihydrospiro[indene-1,5′-oxazolidin]-3′-yl)acetamido)cyclobutyl)azanediyl)diacetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 8.71 (d, J=5.6 Hz, 1H), 7.51 (d, J=14.0 Hz, 1H), 7.36-6.98 (m, 6H), 6.10 (s, 1H), 4.96-4.43 (m, 4H), 4.31 (d, J=25.5 Hz, 1H), 4.05-3.58 (m, 4H), 3.19-2.82 (m, 2H), 2.73-2.53 (m, 4H), 2.56-2.17 (m, 2H). LC-MS (ESI): 626 (M+H)⁺.

### Example 865

### N-{3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclobutyl}glycine

### Example 865A

### (R)-tert-butyl 2-((3-(N-(4-fluorobenzyl)-2-(5-(3-methylureido)-2′,4′-dioxo-2,3-dihydrospiro[indene-1,5′-oxazolidin]-3′-yl)acetamido)cyclobutyl)amino)acetate

The title compound was prepared as described in EXAMPLE 832A, substituting N-(3-aminocyclobutyl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide for 5′-bromo-2′,3′-dihydrospiro[imidazolidine-4,1′-indene]-2,5-dione.

### Example 865B

### N-{3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclobutyl}glycine

The title compound was prepared as described in EXAMPLE 302, substituting (R)-tert-butyl 2-((3-(N-(4-fluorobenzyl)-2-(5-(3-methylureido)-2′,4′-dioxo-2,3-dihydrospiro[indene-1,5′-oxazolidin]-3′-yl)acetamido)cyclobutyl)amino)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 9.27 (s, 1H), 8.79 (d, J=14.7 Hz, 1H), 7.50 (d, J=21.5 Hz, 1H), 7.38-7.05 (m, 6H), 6.22 (s, 1H), 5.03 (q, J=8.3 Hz, 1H), 4.86-4.24 (m, 4H), 3.83 (d, J=14.5 Hz, 2H), 3.63 (d, J=26.7 Hz, 1H), 3.21-2.90 (m, 2H), 2.62 (d, J=2.7 Hz, 4H), 2.57-2.27 (m, 2H). LC-MS (ESI): 568 (M+H)⁺.

### Example 866

### tert-butyl 3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclobutanecarboxylate

The title compound was prepared as described in EXAMPLE 890B. ¹H NMR (400 MHz, DMSO-d₆) δ 8.67 (d, J=2.2 Hz, 1H), 7.53 (d, J=7.1 Hz, 1H), 7.39-7.01 (m, 6H), 6.20-5.91 (m, 1H), 4.84-4.17 (m, 4H), 3.23-2.89 (m, 2H), 2.77-2.55 (m, 4H), 2.57-2.35 (m, 2H), 2.36-2.03 (m, 1H), 1.34 (d, J=1.8 Hz, 9H). LC-MS (ESI): 595 (M+H)⁺.

### Example 867

### methyl {3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclobutyl}carbamate

To a solution of N-(3-aminocyclobutyl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide (60 mg, 0.110 mmol) in 2 mL N,N-dimethylformamide was added N-ethyl-N-isopropylpropan-2-amine (0.058 ml, 0.330 mmol) followed by methyl carbonochloridate (0.012 ml, 0.165 mmol). The reaction mixture was allowed to stir overnight at room temperature, and was diluted with ethyl acetate, and washed with saturated sodium bicarbonate, water, and brine. The combined organic layers were dried with MgSO₄, filtered and concentrated. Silica gel column chromatography provided the title compound. ¹H NMR (400 MHz, DMSO-d₆) δ 8.83-8.53 (m, 1H), 7.52 (d, J=4.8 Hz, 1H), 7.38-6.97 (m, 6H), 6.04 (t, J=4.6 Hz, 1H), 5.09-4.14 (m, 4H), 3.72 (dd, J=48.1, 7.8 Hz, 1H), 3.30 (s, 3H), 3.17-2.89 (m, 2H), 2.61 (dd, J=4.5, 2.9 Hz, 4H), 2.52-2.27 (m, 2H), 2.22-1.92 (m, 3H). LC-MS (ESI): 568 (M+H)⁺.

### Example 868

### ethyl ({3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclobutyl}sulfamoyl)carbamate

The title compound was prepared as described in EXAMPLE 678, substituting N-(3-aminocyclobutyl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide for N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide. ¹H NMR (400 MHz, DMSO-d₆) δ 11.05 (s, 1H), 8.80-8.57 (m, 1H), 8.15 (dd, J=11.3, 7.0 Hz, 1H), 7.51 (d, J=2.5 Hz, 1H), 7.19 (dddd, J=26.4, 20.5, 13.2, 5.0 Hz, 6H), 6.04 (t, J=4.6 Hz, 1H), 4.91-4.17 (m, 4H), 4.14-3.87 (m, 3H), 3.65 (q, J=8.0, 7.2 Hz, 1H), 3.21-2.86 (m, 3H), 2.60 (t, J=4.0 Hz, 4H), 2.53-2.29 (m, 2H), 2.29-2.05 (m, 2H), 1.25-0.95 (m, 3H). LC-MS (ESI): 661 (M+H)⁺.

### Example 869

### 3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclobutanecarboxylic acid

The title compound was prepared as described in EXAMPLE 302, substituting tert-butyl 3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclobutanecarboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 12.20 (s, 1H), 8.67 (d, J=3.0 Hz, 1H), 7.53 (d, J=7.0 Hz, 1H), 7.38-6.97 (m, 6H), 6.05 (d, J=5.9 Hz, 1H), 4.83-4.23 (m, 4H), 3.21-2.88 (m, 2H), 2.78-2.54 (m, 2H), 2.56-2.34 (m, 5H), 2.34-1.98 (m, 2H). LC-MS (ESI): 539 (M+H)⁺.

### Example 870

### 2-[(3′R,4S)-3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

### Example 870A

### (S)-5′-bromo-6′-fluorospiro[imidazolidine-4,1′-indene]-2,3′,5 (2′H)-trione

The mixture of diastereomers of 5′-bromo-6′-fluorospiro[imidazolidine-4,1′-indene]-2,3′,5(2′H)-trione was separated by chiral prep-SFC (Dacel column) to give (R)-5′-bromo-6′-fluorospiro[imidazolidine-4,1′-indene]-2,3′,5(2′H)-trione (eluted first) and (S)-5′-bromo-6′-fluorospiro[imidazolidine-4,1′-indene]-2,3′,5(2′H)-trione (eluted second).

### Example 870B

### 2-((S)-5′-bromo-6′-fluoro-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide

The title compound was prepared as described in EXAMPLE 281F, substituting (S)-5′-bromo-6′-fluorospiro[imidazolidine-4,1′-indene]-2,3′,5(2′H)-trione for (R)-5-bromo-2,3-dihydrospiro[indene-1,5′-oxazolidine]-2′,4′-dione.

### Example 870C

### 2-((1′S,3′S)-5′-bromo-6′-fluoro-3′-hydroxy-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide

The title compound was prepared as described in EXAMPLE 570A, substituting 2-((R)-5′-bromo-6′-fluoro-2,3′,5-trioxo-2′,3′-dihydrospiro[imidazolidine-4,1′-inden]-1-yl)-N-(4-fluorobenzyl)-N—((R)-1,1,1-trifluoropropan-2-yl)acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide.

### Example 870D

### 2-((1′S,3′R)-5′-bromo-3′,6′-difluoro-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide

The title compound was prepared as described in EXAMPLE 514, substituting 2-((1′S,3′S)-5′-bromo-6′-fluoro-3′-hydroxy-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide.

### Example 870E

### 2-[(3′R,4S)-3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 295, substituting 2-((1′S,3′R)-5′-bromo-3′,6′-difluoro-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆, 120° C.) δ 8.66 (s, 1H), 8.12 (s, 1H), 7.89 (s, 1H), 7.83 (d, J=7.0 Hz, 1H), 7.52 (s, 1H), 7.36-7.27 (m, 2H), 7.19 (d, J=10.6 Hz, 1H), 7.09 (t, J=8.7 Hz, 2H), 6.20-5.99 (m, 1H), 5.15 (s, 1H), 4.83-4.75 (m, 3H), 4.58 (d, J=17.7 Hz, 1H), 4.44 (d, J=16.2 Hz, 1H), 4.24 (d, J=16.7 Hz, 1H), 3.07 (td, J=14.2, 6.8 Hz, 1H), 2.65 (d, J=4.7 Hz, 3H), 2.45-2.35 (m, 1H), 1.33 (d, J=7.0 Hz, 3H). MS (ESI⁺) m/z 637 (M+H)⁺.

### Example 871

### 2-{4-[(3′R,4S)-3′,6′-difluoro-1-(2-{(4-fluorobenzyl)[(2S)-1,1,1-trifluoropropan-2-yl]amino}-2-oxoethyl)-2,5-dioxo-2′,3′-dihydro-1H-spiro[imidazolidine-4,1′-inden]-5′-yl]-1H-pyrazol-1-yl}-N,N-dimethylacetamide

The title compound was prepared as described in EXAMPLE 295, substituting 2-((1′S,3′R)-5′-bromo-3′,6′-difluoro-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N,N-dimethyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆, 120° C.) δ 8.67 (s, 1H), 8.07 (s, 1H), 7.87 (s, 1H), 7.82 (d, J=7.1 Hz, 1H), 7.37-7.27 (m, 2H), 7.19 (d, J=10.7 Hz, 1H), 7.09 (t, J=8.7 Hz, 2H), 6.21-5.97 (m, 1H), 5.16 (s, 1H), 5.08 (s, 2H), 4.79 (d, J=17.5 Hz, 1H), 4.58 (d, J=17.9 Hz, 1H), 4.44 (d, J=16.2 Hz, 1H), 4.24 (d, J=16.6 Hz, 1H), 3.07 (td, J=14.2, 6.9 Hz, 1H), 2.95 (s, 6H), 2.45-2.34 (m, 1H), 1.33 (d, J=7.0 Hz, 3H). MS (ESI⁺) m/z 651 (M+H)⁺.

### Example 872

### 2-[(3′S,4R)-3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

### Example 872A

### 2-((R)-5′-bromo-6′-fluoro-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide

The title compound was prepared as described in EXAMPLE 281F, substituting (R)-5′-bromo-6′-fluorospiro[imidazolidine-4,1′-indene]-2,3′,5(2′H)-trione for (R)-5-bromo-2,3-dihydrospiro[indene-1,5′-oxazolidine]-2′,4′-dione.

### Example 872B

### 2-((1′R,3′R)-5′-bromo-6′-fluoro-3′-hydroxy-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide

The title compound was prepared as described in EXAMPLE 570A, substituting 2-((R)-5′-bromo-6′-fluoro-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide.

### Example 872C

### 2-((1′R,3′S)-5′-bromo-3′,6′-difluoro-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide

The title compound was prepared as described in EXAMPLE 514, substituting 2-((1′R,3′R)-5′-bromo-6′-fluoro-3′-hydroxy-2,5-dioxo-2′,3′-dihydrospiro[imidazolidinee-4,1′-inden]-1-yl)-N-(4-fluorobenzyl)-N—((S)-1,1,1-trifluoropropan-2-yl)acetamide for N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide.

### Example 872D

### 2-[(3′S,4R)-3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 295, substituting 2-((1′R,3′S)-5′-bromo-3′,6′-difluoro-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆, 120° C.) δ 8.67 (s, 1H), 8.13 (d, J=1.9 Hz, 1H), 7.89 (s, 1H), 7.83 (d, J=7.0 Hz, 1H), 7.53 (s, 1H), 7.32 (dd, J=8.1, 5.7 Hz, 2H), 7.20 (d, J=10.5 Hz, 1H), 7.10 (t, J=8.8 Hz, 2H), 6.19-5.98 (m, 1H), 5.16 (m, 1H), 4.84-4.69 (m, 3H), 4.58 (d, J=17.4 Hz, 1H), 4.45 (d, J=16.9 Hz, 1H), 4.23 (d, J=17.2 Hz, 1H), 3.07 (td, J=14.1, 6.8 Hz, 1H), 2.65 (d, J=4.7 Hz, 3H), 2.44-2.34 (m, 1H), 1.33 (d, J=7.0 Hz, 3H). MS (ESI⁺) m/z 637 (M+H)⁺.

### Example 873

### 2-{4-[(3′S,4R)-3′,6′-difluoro-1-(2-{(4-fluorobenzyl)[(2S)-1,1,1-trifluoropropan-2-yl]amino}-2-oxoethyl)-2,5-dioxo-2′,3′-dihydro-1H-spiro[imidazolidine-4,1′-inden]-5′-yl]-1H-pyrazol-1-yl}-N,N-dimethylacetamide

The title compound was prepared as described in EXAMPLE 295, substituting 2-((1′R,3′S)-5′-bromo-3′,6′-difluoro-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N,N-dimethyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆, 120° C.) δ 8.65 (s, 1H), 8.07 (d, J=1.8 Hz, 1H), 7.87 (s, 1H), 7.82 (d, J=6.7 Hz, 1H), 7.32 (dd, J=8.4, 5.5 Hz, 2H), 7.19 (d, J=10.5 Hz, 1H), 7.10 (t, J=8.8 Hz, 2H), 6.19-5.98 (m, 1H), 5.16 (s, 1H), 5.07 (s, 2H), 4.80 (d, J=17.6 Hz, 1H), 4.58 (d, J=17.6 Hz, 1H), 4.44 (d, J=16.5 Hz, 1H), 4.23 (d, J=16.8 Hz, 1H), 3.07 (td, J=14.2, 6.8 Hz, 1H), 2.95 (s, 6H), 2.44-2.34 (m, 1H), 1.33 (d, J=7.0 Hz, 3H). MS (ESI⁺) m/z 651 (M+H)⁺.

### Example 874

### ethyl [(4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}piperidin-1-yl)sulfonyl]carbamate

The title compound was prepared as described in EXAMPLE 295, substituting ethyl (4-(2-(5′-bromo-2,5-dioxo-2′,3′-dihydrospiro[imidazolidine-4,1′-inden]-1-yl)-N-(4-fluorobenzyl)acetamido)piperidin-1-yl)sulfonylcarbamate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (501 MHz, methanol-d₄) δ 8.12 (s, 1H), 7.97 (s, 1H), 7.54 (s, 1H), 7.50-7.32 (m, 3H), 7.25 (dd, J=8.4, 5.3 Hz, 1H), 7.13 (t, J=8.6 Hz, 1H), 7.01 (t, J=8.6 Hz, 1H), 4.71-4.56 (m, 3H), 4.46-4.29 (m, 2H), 4.17 (qd, J=7.0, 4.8 Hz, 2H), 3.99 (s, 1H), 3.82 (t, J=13.1 Hz, 2H), 3.17 (td, J=14.9, 14.4, 7.7 Hz, 2H), 3.02 (t, J=10.6 Hz, 1H), 2.91 (s, 1H), 2.80-2.64 (m, 4H), 2.34 (dt, J=13.5, 8.5 Hz, 1H), 1.87-1.62 (m, 5H), 1.28 (td, J=7.2, 4.4 Hz, 3H). MS (ESI⁺) m/z 739 (M+H)⁺.

### Example 875

### ethyl ({4-[(4-fluorobenzyl) {[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]piperidin-1-yl}sulfonyl)carbamate

### Example 875A

### (R)-tert-butyl 4-(2-(5-bromo-2′,4′-dioxo-2,3-dihydrospiro[indene-1,5′-oxazolidin]-3′-yl)-N-(4-fluorobenzyl)acetamido)piperidine-1-carboxylate

The title compound was prepared as described in EXAMPLE 306, substituting (R)-2-(5-bromo-2′,4′-dioxo-2,3-dihydrospiro[indene-1,5′-oxazolidin]-3′-yl)acetic acid for cyclopropanecarboxylic acid and tert-butyl 4-((4-fluorobenzyl)amino)piperidine-1-carboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 875B

### (R)-2-(5-bromo-2′,4′-dioxo-2,3-dihydrospiro[indene-1,5′-oxazolidin]-3′-yl)-N-(4-fluorobenzyl)-N-(piperidin-4-yl)acetamide

The title compound was prepared as described in EXAMPLE 302, substituting (R)-tert-butyl 4-(2-(5-bromo-2′,4′-dioxo-2,3-dihydrospiro[indene-1,5′-oxazolidin]-3′-yl)-N-(4-fluorobenzyl)acetamido)piperidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5-[1,3]oxazolidin]-5-yl]azetidine-1-carboxylate.

### Example 875C

### (R)-ethyl (4-(2-(5-bromo-2′,4′-dioxo-2,3-dihydrospiro[indene-1,5′-oxazolidin]-3′-yl)-N-(4-fluorobenzyl)acetamido)piperidin-1-yl)sulfonylcarbamate

The title compound was prepared as described in EXAMPLE 678, substituting (R)-2-(5-bromo-2′,4′-dioxo-2,3-dihydrospiro[indene-1,5′-oxazolidin]-3′-yl)-N-(4-fluorobenzyl)-N-(piperidin-4-yl)acetamide for N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide.

### Example 875D

### ethyl ({4-[(4-fluorobenzyl) {[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]piperidin-1-yl}sulfonyl)carbamate

The title compound was prepared as described in EXAMPLE 295, substituting (R)-ethyl (4-(2-(5-bromo-2′,4′-dioxo-2,3-dihydrospiro[indene-1,5′-oxazolidin]-3′-yl)-N-(4-fluorobenzyl)acetamido)piperidin-1-yl)sulfonylcarbamate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (500 MHz, methanol-d₄) δ 8.00 (d, J=6.5 Hz, 1H), 7.86 (d, J=5.6 Hz, 1H), 7.58 (s, 1H), 7.51 (dd, J=16.0, 5.5 Hz, 2H), 7.41 (dd, J=8.5, 5.1 Hz, 1H), 7.30-7.23 (m, 1H), 7.14 (t, J=8.6 Hz, 1H), 7.03 (t, J=8.6 Hz, 1H), 4.82-4.71 (m, 1H), 4.66 (d, J=16.2 Hz, 2H), 4.55-4.35 (m, 1H), 4.18 (tt, J=7.1, 3.6 Hz, 2H), 4.14-3.99 (m, 1H), 3.93 (d, J=4.2 Hz, 3H), 3.83 (d, J=12.2 Hz, 2H), 3.29-3.10 (m, 1H), 2.99 (dt, J=49.5, 12.1 Hz, 2H), 2.87-2.74 (m, 1H), 2.66-2.51 (m, 1H), 1.92-1.60 (m, 5H), 1.35-1.20 (m, 3H). MS (ESI⁺) m/z 683 (M+H)+.

### Example 876

### 2-[(1 S,3S)-5-bromo-3-hydroxy-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

### Example 876A

### (S)-5-bromospiro[indene-1,5′-oxazolidine]-2′,3,4′(2H)-trione

(S)-5-Bromo-2,3-dihydrospiro[indene-1,5′-oxazolidine]-2′,4′-dione (2 g, 7.09 mmol) in 60 mL acetone was cooled to −78° C. Ground KMnO₄ (8.96 g, 56.7 mmol) and iron(III) chloride (4.60 g, 28.4 mmol) were added. The mixture was stirred at −78° C. for 1 hour and then 0° C. to room temperature overnight, resulting in a suspension that was filtered. The solid was washed thoroughly with acetone and the filtrate was concentrated and purified by silica gel column chromatography to give the title compound.

### Example 876B

### (1S,3S)-5-bromo-3-hydroxy-2,3-dihydrospiro[indene-1,5′-oxazolidine]-2′,4′-dione

The title compound was prepared as described in EXAMPLE 570A, substituting (S)-5-bromospiro[indene-1,5′-oxazolidine]-2′,3,4′(2H)-trione for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide.

### Example 876C

### 2-[(1 S,3S)-5-bromo-3-hydroxy-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 281F, substituting (1S,3S)-5-bromo-3-hydroxy-2,3-dihydrospiro[indene-1,5′-oxazolidine]-2′,4′-dione for (R)-5-bromo-2,3-dihydrospiro[indene-1,5′-oxazolidine]-2′,4′-dione. ¹H NMR (400 MHz, DMSO-d₆) δ 7.65 (d, J=1.9 Hz, 1H), 7.56 (dd, J=8.2, 2.0 Hz, 1H), 7.41-7.28 (m, 3H), 7.18-7.04 (m, 2H), 5.48 (s, 1H), 5.38 (q, J=6.4 Hz, 1H), 5.17 (s, 1H), 4.87-4.27 (m, 4H), 2.87 (d, J=3.2 Hz, 1H), 2.42 (dd, J=14.5, 6.5 Hz, 1H), 1.37 (d, J=7.0 Hz, 3H). MS (ESI⁺) m/z 559 (M−H)⁺.

### Example 877

### 2-(5-chloro-4-cyano-2′,4′-dioxo-2,3-dihydro-3′H-spiro[indene-1,5′-[1,3]oxazolidin]-3′-yl)-N-(4-fluorobenzyl)-N-[(2S)-1,1,1-trifluoropropan-2-yl]acetamide

### Example 877A

### 3-chloro-2-iodobenzoic acid

To a solution of 2-amino-3-chlorobenzoic acid (24 g, 140 mmol) in dimethylsulfoxide (50 ml) and 30% sulfuric acid (150 ml, 140 mmol) was added sodium nitrite (14.48 g, 210 mmol) in water (50 ml) dropwise at 0° C. The mixture was stirred at 0° C. for 1 hour, at which time a solution of KI (58.0 g, 350 mmol) in water (50 ml) was added dropwise. The ice-water bath was removed and the mixture was stirred for another 2 hours. Ethyl acetate (800 mL) was added and the mixture was washed with 2N aqueous Na₂SO₃ three times. The organic layer was dried over Na₂SO₄, filtered through a Büchner funnel and concentrated to afford the title compound.

### Example 877B

### 3-chloro-2-iodo-N-methoxy-N-methylbenzamide

To a solution of 3-chloro-2-iodobenzoic acid (39.5 g, 140 mmol) in methylene chloride (500 ml) and N,N-dimethylformamide (1.083 ml, 13.98 mmol) was added thionyl chloride (20.41 ml, 280 mmol) dropwise. The mixture was stirred for 1 hour at room temperature and concentrated under reduced pressure to give 3-(3-chloro-2-iodophenyl)propanoyl chloride which was used directly in next step. To a stirred solution of N,N,O-trimethylhydroxylamine hydrochloride (20.47 g, 183 mmol) and 3-chloro-2-iodobenzoyl chloride (46 g, 153 mmol) in dry methylene chloride (500 ml) was added triethylamine (74.6 ml, 535 mmol) dropwise at 0° C. The mixture was stirred for 4 hours at room temperature and then poured into water. The organic layer was separated, washed with water and brine, dried over Na₂SO₄, filtered and concentrated to afford the title compound.

### Example 877C

### 3-chloro-2-iodobenzaldehyde

To a solution of 3-chloro-2-iodo-N-methoxy-N-methylbenzamide (49.8 g, 153 mmol) in tetrahydrofuran (500 ml) was added diisobutylaluminum hydride (500 ml, 500 mmol) dropwise at −78° C. The mixture was stirred at −78° C. for 4 hours and quenched by adding 50 ml of methanol carefully. The mixture was partitioned between ethyl acetate and water. The aqueous phase was extracted with ethyl acetate. The combined organic layers were washed with 1N aqueous HC 1, saturated sodium bicarbonate and brine, dried over Na₂SO₄, filtered and concentrated to give the title compound.

### Example 877D

### 3-(3-chloro-2-iodophenyl)propanoic acid

To a flask charged with triethylamine (16.70 ml, 120 mmol) was added formic acid (11.49 ml, 299 mmol) portionwise, and the mixture was stirred for 15 minutes at room temperature. The mixture was diluted with N,N-dimethylformamide (75 ml). To the solution was added 3-chloro-2-iodobenzaldehyde (26.6 g, 100 mmol) and 2, 2-dimethyl-1,3-dioxane-4, 6-dione (14.39 g, 100 mmol). The resulting mixture was stirred at 100° C. for 48 hours. The mixture was poured into ice/concentrated HCl (800 ml), and extracted with methylene chloride (2×500 ml). The organic layer was washed with 1N aqueous sodium hydroxide (2×200 ml). The combined aqueous layers were acidified to pH 2 with concentrated hydrochloride acid and extracted with ethyl acetate (2×500 ml). The organic layer was dried with sodium sulfate, filtered, and concentrated under reduced pressure to give the title compound.

### Example 877E

### 5-chloro-4-iodo-2,3-dihydro-1H-inden-1-one

To a solution of 3-(3-chloro-2-iodophenyl)propanoic acid (17 g, 54.7 mmol) in methylene chloride (100 ml) with N,N-dimethylformamide ((4.00 g, 54.7 mmol) was added thionyl chloride (4.00 ml, 54.7 mmol) dropwise. The mixture was stirred for 1 hour at room temperature and then concentrated under reduced pressure to dryness. The residue was dissolved in methylene chloride (100 ml) and was added dropwise to a refluxing suspension of aluminum chloride (29.2 g, 219 mmol) in methylene chloride (100 ml). The mixture was refluxed for 90 minutes and poured into ice/concentrated HCl and extracted with methylene chloride (3×100 mL). The organic layers were combined and concentrated. The residue was purified by silica gel column chromatography (on silica gel, eluted with petroleum ether:ethyl acetate, 5:1) to afford the title compound.

### Example 877F

### 5-chloro-4-iodo-1-((trimethylsilyl)oxy)-2,3-dihydro-1H-indene-1-carbonitrile

To a mixture of 5-chloro-4-iodo-2,3-dihydro-1H-inden-1-one (5.6 g, 19.15 mmol) and trimethylsilanecarbonitrile (2.87 ml, 22.97 mmol) was added N-morpholine oxide (2.243 g, 19.15 mmol). The resulting mixture was stirred at room temperature for 16 hours and then quenched with water (5 ml). The mixture was extracted with methylene chloride (2×100 mL). The organic layer was dried over Na₂SO₄, filtered through a Büchner funnel, and concentrated to afford the title compound.

### Example 877G

### ethyl 5-chloro-1-hydroxy-4-iodo-2,3-dihydro-1H-indene-1-carbimidate

A three neck 3 L flask was fitted with hastalloy thermocouple, HCl gas in (diffussor) and HCl out, vented out the back of the hood. 5-Chloro-4-iodo-1-((trimethylsilyl)oxy)-2,3-dihydro-1H-indene-1-carbonitrile (7.5 g, 19.15 mmol) was taken up in ethanol (100 ml) and hydrogen chloride (13.96 g, 383 mmol) was bubbled in carefully (backflow preventer), emptying the lecture bottle over 2 hours. The internal temperature was kept below 25° C. with an ice bath. The mixture was stirred overnight and then concentrated. The crude material was washed with ether three times and placed on high vacuum overnight to afford the title compound.

### Example 877H

### 5-chloro-4-iodo-2,3-dihydrospiro[indene-1,5′-oxazolidine]-2′,4′-dione

To a suspension of ethyl 5-chloro-1-hydroxy-4-iodo-2,3-dihydro-1H-indene-1-carbimidate (5.7 g, 14.18 mmol) in tetrahydrofuran (50 ml) was added triethylamine (5.93 ml, 42.5 mmol) at 0° C. over an ice bath followed by addition of triphosgene (4.21 g, 14.18 mmol). The mixture was warmed to room temperature and stirred for 2 hours. The mixture was diluted with concentrated HCl, stirred overnight and then extracted with ethyl acetate. The aqueous phase was extracted with ethyl acetate (3×100 ml). The combined organic phases were dried with sodium sulfate, filtered and concentrated. The residue was purified by silica gel column chromatography (eluted with petroleum ether:ethyl acetate, 2:1) to afford the title compound.

### Example 8771

### 5-chloro-2′,4′-dioxo-2,3-dihydrospiro[indene-1,5′-oxazolidine]-4-carbonitrile

A solution of 5-chloro-4-iodo-2,3-dihydrospiro[indene-1,5′-oxazolidine]-2′,4′-dione (3.0 g, 8.25 mmol), dicyanozinc (1066 mg, 9.08 mmol) and tetrakis(triphenylphosphine)palladium(0) (954 mg, 0.825 mmol) in N,N-dimethylformamide (15 ml) was degassed with argon for 5 minutes. The suspension was heated in a Biotage Initiator® microwave at 100° C. for 2 hours. The mixture was diluted with ethyl acetate and filtered. The filtrate was partitioned between water and ethyl acetate and the layers were separated. The organic layer was washed with water, dried over Na₂SO₄, filtered and concentrated in vacuo. The crude mixture was purified by silica gel column chromatography to afford the title compound.

### Example 877J

### 2-(5-chloro-4-cyano-2′,4′-dioxo-2,3-dihydro-3′H-spiro[indene-1,5′-[1,3]oxazolidin]-3′-yl)-N-(4-fluorobenzyl)-N-[(2S)-1,1,1-trifluoropropan-2-yl]acetamide

The title compound was prepared as described in EXAMPLE 281F, substituting 5-chloro-2′,4′-dioxo-2,3-dihydrospiro[indene-1,5′-oxazolidine]-4-carbonitrile for (R)-5-bromo-2,3-dihydrospiro[indene-1,5′-oxazolidine]-2′,4′-dione. ¹H NMR (400 MHz, DMSO-d₆) δ 7.86-7.66 (m, 2H), 7.34 (dd, J=8.6, 5.5 Hz, 1.3H), 7.21 (td, J=8.6, 8.1, 2.4 Hz, 2H), 7.09 (td, J=8.7, 5.3 Hz, 0.7H), 5.36 (dq, J=15.5, 7.5 Hz, 0.7H), 5.09-4.67 (m, 2.3H), 4.64-4.36 (m, 1.3H), 4.18 (dd, J=17.1, 1.9 Hz, 0.7H), 3.36 (ddt, J=17.5, 8.6, 6.4 Hz, 1H), 3.16-3.26 (m, 1H), 2.83-2.54 (m, 2H), 1.33 (ddd, J=18.0, 7.0, 3.0 Hz, 3H). MS (ESI⁺) m/z 546 (M+Na)⁺.

### Example 880

### tert-butyl 4-{[(5′-bromo-2, 5-dioxo-2′,3′-dihydro-1H-spiro[imidazolidine-4,1′-inden]-1-yl)acetyl](4-fluorobenzyl)amino}piperidine-1-carboxylate

### Example 880A

### tert-butyl 4-(2-bromo-N-(4-fluorobenzyl)acetamido)piperidine-1-carboxylate

The title compound was prepared as described in EXAMPLE 281B, substituting tert-butyl 4-((4-fluorobenzyl)amino)piperidine-1-carboxylate for (S)-1,1,1-trifluoro-N-(4-fluorobenzyl)propan-2-amine.

### Example 880B

### tert-butyl 4-{[(5′-bromo-2, 5-dioxo-2′,3′-dihydro-1H-spiro[imidazolidine-4,1′-inden]-1-yl)acetyl](4-fluorobenzyl)amino}piperidine-1-carboxylate

The title compound was prepared as described in EXAMPLE 281F, substituting (S)-5′-bromo-2′,3′-dihydrospiro[imidazolidine-4,1′-indene]-2,5-dione for (R)-5-bromo-2,3-dihydrospiro[indene-1,5′-oxazolidine]-2′,4′-dione and tert-butyl 4-(2-bromo-N-(4-fluorobenzyl)acetamido)piperidine-1-carboxylate for (S)-2-bromo-N-(4-fluorobenzyl)-N-(1,1,1-trifluoropropan-2-yl)acetamide. ¹H NMR (400 MHz, methanol-d₄) δ 7.47 (d, J=1.7 Hz, 1H), 7.41-7.33 (m, 2H), 7.28 (dd, J=8.2, 4.9 Hz, 1H), 7.24-7.16 (m, 1H), 7.09 (t, J=8.7 Hz, 1H), 7.02-6.92 (m, 1H), 4.69-4.52 (m, 3H), 4.48-4.27 (m, 2H), 4.16-3.91 (m, 2H), 3.10 (ddd, J=13.4, 7.5, 3.3 Hz, 2H), 2.69 (dddd, J=13.3, 11.4, 8.2, 4.9 Hz, 2H), 2.31 (dtd, J=13.6, 8.6, 7.1 Hz, 1H), 1.78-1.48 (m, 4H), 1.40 (d, J=6.0 Hz, 9H). MS (ESI⁺) m/z 630 (M+H)⁺.

### Example 881

### N-(4-fluorobenzyl)-2-[(1 S,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-((1S,3S)-5-bromo-3-hydroxy-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.09 (s, 1H), 7.83 (s, 1H), 7.63 (d, J=1.6 Hz, 1H), 7.58-7.50 (m, 2H), 7.42-7.23 (m, 3H), 7.13 (t, J=8.8 Hz, 2H), 5.47-5.10 (m, 3H), 4.93-4.29 (m, 6H), 2.86 (m, 1H), 2.65 (d, J=4.7 Hz, 3H), 2.42 (m, 1H), 1.38 (d, J=7.0 Hz, 3H). MS (ESI⁺) m/z 618 (M+H)⁺.

### Example 882

### ethyl ({4-[{[(4S)-5′-amino-2,5-dioxo-2′,3′-dihydro-1H-spiro[imidazolidine-4,1′-inden]-1-yl]acetyl}(4-fluorobenzyl)amino]piperidin-1-yl}sulfonyl)carbamate

### Example 882A

### (S)-tert-butyl 4-(2-(5′-bromo-2,5-dioxo-2′,3′-dihydrospiro[imidazolidine-4,1′-inden]-1-yl)-N-(4-fluorobenzyl)acetamido)piperidine-1-carboxylate

The title compound was prepared as described in EXAMPLE 281F, substituting (S)-5′-bromo-2′,3′-dihydrospiro[imidazolidine-4,1′-indene]-2,5-dione for (R)-5-bromo-2,3-dihydrospiro[indene-1,5′-oxazolidine]-2′,4′-dione and tert-butyl 4-(2-bromo-N-(4-fluorobenzyl)acetamido)piperidine-1-carboxylate for (S)-2-bromo-N-(4-fluorobenzyl)-N-(1,1,1-trifluoropropan-2-yl)acetamide.

### Example 882B

### (S)-tert-butyl 4-(2-(5′-((diphenylmethylene)amino)-2,5-dioxo-2′,3′-dihydrospiro[imidazolidine-4,1′-inden]-1-yl)-N-(4-fluorobenzyl)acetamido)piperidine-1-carboxylate

The title compound was prepared as described in EXAMPLE 284D, substituting (S)-tert-butyl 4-(2-(5′-bromo-2,5-dioxo-2′,3′-dihydrospiro[imidazolidine-4,1′-inden]-1-yl)-N-(4-fluorobenzyl)acetamido)piperidine-1-carboxylate for N-benzyl-2-((S)-5-bromo-2′,4′-dioxo-2,3-dihydrospiro[indene-1,5′-oxazolidin]-3′-yl)-N—((S)-1-cyclopropylethyl)acetamide.

### Example 882C

### (S)-2-(5′-((diphenylmethylene)amino)-2,5-dioxo-2′,3′-dihydrospiro[imidazolidine-4,1′-inden]-1-yl)-N-(4-fluorobenzyl)-N-(piperidin-4-yl)acetamide

The title compound was prepared as described in EXAMPLE 302, substituting (S)-tert-butyl 4-(2-(5′-((diphenylmethylene)amino)-2,5-dioxo-2′,3′-dihydrospiro[imidazolidine-4,1′-inden]-1-yl)-N-(4-fluorobenzyl)acetamido)piperidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 882D

### (S)-ethyl (4-(2-(5′-((diphenylmethylene)amino)-2,5-dioxo-2′,3′-dihydrospiro[imidazolidine-4, 1′-inden]-1-yl)-N-(4-fluorobenzyl)acetamido)piperidin-1-yl)sulfonylcarbamate

The title compound was prepared as described in EXAMPLE 678, substituting (S)-2-(5′-((diphenylmethylene)amino)-2,5-dioxo-2′,3′-dihydrospiro[imidazolidine-4,1′-inden]-1-yl)-N-(4-fluorobenzyl)-N-(piperidin-4-yl)acetamide for N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide.

### Example 882E

### ethyl ({4-[{[(4S)-5′-amino-2,5-dioxo-2′,3′-dihydro-1H-spiro[imidazolidine-4,1′-inden]-1-yl]acetyl}(4-fluorobenzyl)amino]piperidin-1-yl}sulfonyl)carbamate

The title compound was prepared as described in EXAMPLE 284E, substituting (S)-ethyl (4-(2-(5′-((diphenylmethylene)amino)-2,5-dioxo-2′,3′-dihydrospiro[imidazolidine-4,1′-inden]-1-yl)-N-(4-fluorobenzyl)acetamido)piperidin-1-yl)sulfonylcarbamate for N—((S)-1-cyclopropylethyl)-2-((S)-5-((diphenylmethylene)amino)-2′,4′-dioxo-2,3-dihydrospiro[indene-1,5′-oxazolidin]-3′-yl)-N-(4-fluorobenzyl)acetamide. ¹H NMR (400 MHz, methanol-d₄) δ 7.51 (dd, J=8.2, 3.7 Hz, 1H), 7.39 (dd, J=8.5, 5.2 Hz, 1H), 7.28 (d, J=1.9 Hz, 1H), 7.25-7.17 (m, 2H), 7.10 (t, J=8.7 Hz, 1H), 7.04-6.93 (m, 1H), 4.70-4.55 (m, 3H), 4.43-4.25 (m, 2H), 4.14 (qd, J=8.5, 7.8, 2.6 Hz, 2H), 3.88-3.74 (m, 2H), 3.25-3.07 (m, 2H), 3.06-2.95 (m, 1H), 2.89 (tt, J=12.6, 2.7 Hz, 1H), 2.74 (dddd, J=13.2, 10.1, 8.1, 5.0 Hz, 1H), 2.36 (dq, J=13.5, 7.9 Hz, 1H), 1.88-1.60 (m, 4H), 1.32-1.16 (m, 3H). MS (ESI⁺) m/z 617 (M+H)⁺.

### Example 883

### ethyl ({3,3-difluoro-4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]pyrrolidin-1-yl}sulfonyl)carbamate

### Example 883A

### tert-butyl 3,3-difluoro-4-((4-fluorobenzyl)amino)pyrrolidine-1-carboxylate

The title compound was prepared as described in EXAMPLE 283A, substituting tert-butyl 4-amino-3,3-difluoropyrrolidine-1-carboxylate for (S)-1-cyclopropylethanamine and 4-fluorobenzaldehyde for benzaldehyde.

### Example 883B

### tert-butyl 4-(2-bromo-N-(4-fluorobenzyl)acetamido)-3,3-difluoropyrrolidine-1-carboxylate

The title compound was prepared as described in EXAMPLE 281B, substituting tert-butyl 3,3-difluoro-4-((4-fluorobenzyl)amino)pyrrolidine-1-carboxylate for (S)-1,1,1-trifluoro-N-(4-fluorobenzyl)propan-2-amine.

### Example 883C

### tert-butyl 4-(2-((S)-5′-bromo-2,5-dioxo-2′,3′-dihydrospiro[imidazolidine-4,1′-inden]-1-yl)-N-(4-fluorobenzyl)acetamido)-3,3-difluoropyrrolidine-1-carboxylate

The title compound was prepared as described in EXAMPLE 281F, substituting (S)-5′-bromo-2′,3′-dihydrospiro[imidazolidine-4,1′-indene]-2,5-dione for (R)-5-bromo-2,3-dihydrospiro[indene-1,5′-oxazolidine]-2′,4′-dione and tert-butyl 4-(2-((S)-5′-bromo-2,5-dioxo-2′,3′-dihydrospiro[imidazolidine-4,1′-inden]-1-yl)-N-(4-fluorobenzyl)acetamido)-3,3-difluoropyrrolidine-1-carboxylate for (S)-2-bromo-N-(4-fluorobenzyl)-N-(1,1,1-trifluoropropan-2-yl)acetamide.

### Example 883D

### tert-butyl 3,3-difluoro-4-(N-(4-fluorobenzyl)-2-((S)-5′-(1-methyl-1H-pyrazol-4-yl)-2,5-dioxo-2′,3′-dihydrospiro[imidazolidine-4,1′-inden]-1-yl)acetamido)pyrrolidine-1-carboxylate

The title compound was prepared as described in EXAMPLE 295, substituting tert-butyl 4-(2-((S)-5′-bromo-2,5-dioxo-2′,3′-dihydrospiro[imidazolidine-4, 1′-inden]-1-yl)-N-(4-fluorobenzyl)acetamido)-3,3-difluoropyrrolidine-1-carboxylate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol.

### Example 883E

### N-(4,4-difluoropyrrolidin-3-yl)-N-(4-fluorobenzyl)-2-((S)-5′-(1-methyl-1H-pyrazol-4-yl)-2,5-dioxo-2′,3′-dihydrospiro[imidazolidine-4,1′-inden]-1-yl)acetamide

The title compound was prepared as described in EXAMPLE 302, substituting tert-butyl 3,3-difluoro-4-(N-(4-fluorobenzyl)-2-((S)-5′-(1-methyl-1H-pyrazol-4-yl)-2,5-dioxo-2′,3′-dihydrospiro[imidazolidine-4,1′-inden]-1-yl)acetamido)pyrrolidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 883F

### ethyl ({3,3-difluoro-4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]pyrrolidin-1-yl}sulfonyl)carbamate

The title compound was prepared as described in EXAMPLE 678, substituting N-(4,4-difluoropyrrolidin-3-yl)-N-(4-fluorobenzyl)-2-((S)-5′-(1-methyl-1H-pyrazol-4-yl)-2,5-dioxo-2′,3′-dihydrospiro[imidazolidine-4,1′-inden]-1-yl)acetamide for N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide. ¹H NMR (400 MHz, methanol-d₄) δ 7.92 (s, 1H), 7.78 (s, 1H), 7.47 (d, J=1.4 Hz, 1H), 7.46-7.26 (m, 4H), 7.12 (t, J=8.6 Hz, 1H), 7.05-6.92 (m, 1H), 4.81 (m, 4H), 4.50 (dd, J=16.7, 7.1 Hz, 1H), 4.32 (dd, J=16.8, 6.0 Hz, 1H), 4.12 (dtd, J=14.1, 7.8, 7.1, 4.1 Hz, 2H), 3.89 (s, 3H), 3.86-3.79 (m, 2H), 3.70 (td, J=10.6, 8.8, 4.7 Hz, 1H), 3.13 (ddd, J=13.7, 8.3, 6.0 Hz, 2H), 2.69 (ddd, J=13.3, 8.0, 5.2 Hz, 1H), 2.30 (dt, J=14.2, 7.7 Hz, 1H), 1.30-1.14 (m, 3H). MS (ESI⁺) m/z 704 (M+H)⁺.

### Example 884

### ethyl ({3,3-difluoro-4-[(4-fluorobenzyl) {[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]acetyl}amino]pyrrolidin-1-yl}sulfonyl)carbamate

### Example 884A

### tert-butyl 3,3-difluoro-4-(N-(4-fluorobenzyl)-2-((S)-5′-(1-(2-(methylamino)-2-oxoethyl)-1H-pyrazol-4-yl)-2,5-dioxo-2′,3′-dihydrospiro[imidazolidine-4,1′-inden]-1-yl)acetamido)pyrrolidine-1-carboxylate

The title compound was prepared as described in EXAMPLE 295, substituting tert-butyl 4-(2-((S)-5′-bromo-2,5-dioxo-2′,3′-dihydrospiro[imidazolidine-4,1′-inden]-1-yl)-N-(4-fluorobenzyl)acetamido)-3,3-difluoropyrrolidine-1-carboxylate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol.

### Example 884B

### N-(4,4-difluoropyrrolidin-3-yl)-N-(4-fluorobenzyl)-2-((S)-5′-(1-(2-(methylamino)-2-oxoethyl)-1H-pyrazol-4-yl)-2,5-dioxo-2′,3′-dihydrospiro[imidazolidine-4,1′-inden]-1-yl)acetamide

The title compound was prepared as described in EXAMPLE 302, substituting tert-butyl 3,3-difluoro-4-(N-(4-fluorobenzyl)-2-((S)-5′-(1-(2-(methylamino)-2-oxoethyl)-1H-pyrazol-4-yl)-2,5-dioxo-2′,3′-dihydrospiro[imidazolidine-4,1′-inden]-1-yl)acetamido)pyrrolidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 884C

### ethyl ({3,3-difluoro-4-[(4-fluorobenzyl) {[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]acetyl}amino]pyrrolidin-1-yl}sulfonyl)carbamate

The title compound was prepared as described in EXAMPLE 678, substituting N-(4,4-difluoropyrrolidin-3-yl)-N-(4-fluorobenzyl)-2-((S)-5′-(1-(2-(methylamino)-2-oxoethyl)-1H-pyrazol-4-yl)-2,5-dioxo-2′,3′-dihydrospiro[imidazolidine-4,1′-inden]-1-yl)acetamide for N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide. ¹H NMR (400 MHz, methanol-d₄) δ 8.02 (s, 1H), 7.88 (s, 1H), 7.55-7.48 (m, 1H), 7.46 (d, J=8.2 Hz, 1H), 7.41-7.29 (m, 2H), 7.20 (s, 1H), 7.13 (t, J=8.6 Hz, 1H), 7.06-6.92 (m, 1H), 4.91-4.71 (m, 6H), 4.51 (dd, J=16.8, 6.9 Hz, 1H), 4.34 (dd, J=16.7, 6.3 Hz, 1H), 4.12 (dq, J=11.4, 7.1 Hz, 2H), 4.03-3.79 (m, 2H), 3.72 (dt, J=11.0, 7.0 Hz, 1H), 3.21-3.06 (m, 2H), 2.74 (s, 3H), 2.73-2.65 (m, 1H), 2.31 (dt, J=14.3, 7.6 Hz, 1H), 1.33-1.08 (m, 3H). MS (ESI⁺) m/z 761 (M+H)⁺.

### Example 885

### ethyl ({3,3-difluoro-4-[(4-fluorobenzyl){[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]pyrrolidin-1-yl}sulfonyl)carbamate

### Example 885A

### tert-butyl 4-(2-((R)-5-bromo-2′,4′-dioxo-2,3-dihydrospiro[indene-1,5′-oxazolidin]-3′-yl)-N-(4-fluorobenzyl)acetamido)-3,3-difluoropyrrolidine-1-carboxylate

The title compound was prepared as described in EXAMPLE 281F, substituting (R)-5-bromo-2,3-dihydrospiro[indene-1,5′-oxazolidine]-2′,4′-dione for (R)-5-bromo-2,3-dihydrospiro[indene-1,5′-oxazolidine]-2′,4′-dione and tert-butyl 4-(2-bromo-N-(4-fluorobenzyl)acetamido)-3,3-difluoropyrrolidine-1-carboxylate for (S)-2-bromo-N-(4-fluorobenzyl)-N-(1,1,1-trifluoropropan-2-yl)acetamide.

### Example 885B

### tert-butyl 3,3-difluoro-4-(N-(4-fluorobenzyl)-2-((R)-5-(1-methyl-1H-pyrazol-4-yl)-2′,4′-dioxo-2,3-dihydrospiro[indene-1,5′-oxazolidin]-3′-yl)acetamido)pyrrolidine-1-carboxylate

The title compound was prepared as described in EXAMPLE 295, substituting tert-butyl 4-(2-((R)-5-bromo-2′,4′-dioxo-2,3-dihydrospiro[indene-1,5′-oxazolidin]-3′-yl)-N-(4-fluorobenzyl)acetamido)-3,3-difluoropyrrolidine-1-carboxylate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol.

### Example 885C

### N-(4,4-difluoropyrrolidin-3-yl)-N-(4-fluorobenzyl)-2-((R)-5-(1-methyl-1H-pyrazol-4-yl)-2′,4′-dioxo-2,3-dihydrospiro[indene-1,5′-oxazolidin]-3′-yl)acetamide

The title compound was prepared as described in EXAMPLE 302, substituting tert-butyl 3,3-difluoro-4-(N-(4-fluorobenzyl)-2-((R)-5-(1-methyl-1H-pyrazol-4-yl)-2′,4′-dioxo-2,3-dihydrospiro[indene-1,5′-oxazolidin]-3′-yl)acetamido)pyrrolidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 885D

### ethyl ({3,3-difluoro-4-[(4-fluorobenzyl){[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]pyrrolidin-1-yl}sulfonyl)carbamate

The title compound was prepared as described in EXAMPLE 678, substituting N-(4,4-difluoropyrrolidin-3-yl)-N-(4-fluorobenzyl)-2-((R)-5-(1-methyl-1H-pyrazol-4-yl)-2′,4′-dioxo-2,3-dihydrospiro[indene-1,5′-oxazolidin]-3′-yl)acetamide for N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide. ¹H NMR (400 MHz, methanol-d₄) δ 7.97 (s, 1H), 7.83 (s, 1H), 7.60-7.53 (m, 1H), 7.52-7.42 (m, 2H), 7.37 (dd, J=8.1, 5.1 Hz, 2H), 7.21 (s, 1H), 7.18-7.10 (m, 1H), 7.02 (d, J=8.6 Hz, 1H), 4.81 (m, 4H), 4.72-4.54 (m, 1H), 4.45 (t, J=17.0 Hz, 1H), 4.25-4.07 (m, 2H), 3.93 (d, J=22.5 Hz, 5H), 3.81-3.68 (m, 1H), 3.27-3.18 (m, 1H), 3.12 (qt, J=8.6, 3.8 Hz, 1H), 2.84-2.69 (m, 1H), 2.54 (ddd, J=13.6, 7.6, 3.7 Hz, 1H), 1.19 (td, J=7.1, 3.6 Hz, 3H). MS (ESI⁺) m/z 705 (M+H)⁺.

### Example 886

### ethyl ({3,3-difluoro-4-[(4-fluorobenzyl){[(1R)-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]acetyl}amino]pyrrolidin-1-yl}sulfonyl)carbamate

### Example 886A

### tert-butyl 3,3-difluoro-4-(N-(4-fluorobenzyl)-2-((R)-5-(1-(2-(methylamino)-2-oxoethyl)-1H-pyrazol-4-yl)-2′,4′-dioxo-2,3-dihydrospiro[indene-1,5′-oxazolidin]-3′-yl)acetamido)pyrrolidine-1-carboxylate

The title compound was prepared as described in EXAMPLE 295, substituting tert-butyl 3,3-difluoro-4-(N-(4-fluorobenzyl)-2-((R)-5-(1-(2-(methylamino)-2-oxoethyl)-1H-pyrazol-4-yl)-2′,4′-dioxo-2,3-dihydrospiro[indene-1,5′-oxazolidin]-3′-yl)acetamido)pyrrolidine-1-carboxylate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol.

### Example 886B

### N-(4,4-difluoropyrrolidin-3-yl)-N-(4-fluorobenzyl)-2-((R)-5-(1-(2-(methylamino)-2-oxoethyl)-1H-pyrazol-4-yl)-2′,4′-dioxo-2,3-dihydrospiro[indene-1,5′-oxazolidin]-3′-yl)acetamide

The title compound was prepared as described in EXAMPLE 302, substituting tert-butyl 3,3-difluoro-4-(N-(4-fluorobenzyl)-2-((R)-5-(1-(2-(methylamino)-2-oxoethyl)-1H-pyrazol-4-yl)-2′,4′-dioxo-2,3-dihydrospiro[indene-1,5′-oxazolidin]-3′-yl)acetamido)pyrrolidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 886C

### ethyl ({3,3-difluoro-4-[(4-fluorobenzyl){[(1R)-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]acetyl}amino]pyrrolidin-1-yl}sulfonyl)carbamate

The title compound was prepared as described in EXAMPLE 678, substituting N-(4,4-difluoropyrrolidin-3-yl)-N-(4-fluorobenzyl)-2-((R)-5-(1-(2-(methylamino)-2-oxoethyl)-1H-pyrazol-4-yl)-2′,4′-dioxo-2,3-dihydrospiro[indene-1,5′-oxazolidin]-3′-yl)acetamide for N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide. ¹H NMR (400 MHz, methanol-d₄) δ 8.08 (s, 1H), 7.92 (s, 1H), 7.57 (s, 1H), 7.53-7.43 (m, 2H), 7.37 (dd, J=8.4, 5.2 Hz, 2H), 7.25-6.98 (m, 2H), 4.81 (m, 9H), 4.73-4.56 (m, 1H), 4.45 (t, J=17.0 Hz, 1H), 4.24-4.04 (m, 2H), 4.03-3.81 (m, 2H), 3.81-3.65 (m, 1H), 3.26-3.18 (m, 1H), 3.11 (ddd, J=15.6, 8.5, 3.7 Hz, 1H), 2.85-2.76 (m, 1H), 2.55 (td, J=10.9, 9.3, 4.6 Hz, 1H), 1.33-1.05 (m, 3H). MS (ESI⁺) m/z 762 (M+H)⁺.

### Example 887

### N-(4-fluorobenzyl)-2-[(1S)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide

N-(4-Fluorobenzyl)-2-[(1 S,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide (60 mg, 0.097 mmol) in 1.5 mL dichloromethane was cooled to −78° C. Bis(2-methoxyethyl)aminosulfur trifluoride (35.2 μl, 0.194 mmol) was added dropwise via syringe. The mixture was stirred at −78° C. for 2 hours, and 0.5 mL methanol was added. The mixture was allowed to warm from −78° C. to room temperature and was diluted with dichloromethane, washed with saturated sodium bicarbonate and brine, and dried over sodium sulfate. Filtration, concentration and silica gel column chromatography provided the title compound. ¹H NMR (400 MHz, DMSO-d₆) δ 8.15 (s, 1H), 7.89 (s, 1H), 7.81-7.68 (m, 2H), 7.57-7.45 (m, 2H), 7.33 (dd, J=8.5, 5.3 Hz, 2H), 7.12 (tt, J=9.5, 2.2 Hz, 2H), 6.11 (ddd, J=56.2, 6.4, 2.4 Hz, 1H), 5.30-5.09 (m, 1H), 4.93-4.31 (m, 6H), 3.26-2.90 (m, 1H), 2.78-2.54 (m, 4H), 1.37 (d, J=7.0 Hz, 3H). MS (ESI⁺) m/z 620 (M+H)⁺.

### Example 888

### 2-[(1S)-5-bromo-3-fluoro-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

### Example 888A

### (1S,3S)-5-bromo-3-hydroxy-2,3-dihydrospiro[indene-1,5′-oxazolidine]-2′,4′-dione

The title compound was prepared as described in EXAMPLE 570A, substituting (S)-5-bromospiro[indene-1,5′-oxazolidine]-2′,3,4′(2H)-trione for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide.

### Example 888B

### 2-((1 S,3S)-5-bromo-3-hydroxy-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide

The title compound was prepared as described in EXAMPLE 281F, substituting (1S,3S)-5-bromo-3-hydroxy-2,3-dihydrospiro[indene-1,5′-oxazolidine]-2′,4′-dione for (R)-5-bromo-2,3-dihydrospiro[indene-1,5′-oxazolidine]-2′,4′-dione.

### Example 888C

### 2-[(1S)-5-bromo-3-fluoro-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 887, substituting 2-((1S,3S)-5-bromo-3-hydroxy-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for N-(4-fluorobenzyl)-2-[(1 S,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide. ¹H NMR (400 MHz, DMSO-d₆) δ 8.00-7.67 (m, 2H), 7.58-7.40 (m, 1H), 7.32 (dd, J=8.4, 5.4 Hz, 2H), 7.12 (t, J=8.6 Hz, 2H), 6.20 (m, 1H), 5.2 (m, 1H), 6.59-5.76 (m, 1H), 4.98-4.24 (m, 4H), 3.15 (ddd, J=20.7, 15.8, 6.5 Hz, 1H), 2.66 (ddd, J=21.9, 15.8, 2.5 Hz, 1H), 1.37 (d, J=7.1 Hz, 3H). MS (ESI⁺) m/z 559 (M−H)⁺.

### Example 889

### N-(4-fluorobenzyl)-N-(3-methoxycyclobutyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

### Example 889A

### N-(4-fluorobenzyl)-3-methoxycyclobutanamine

The title compound was prepared as described in EXAMPLE 283A, substituting 3-methoxycyclobutanamine hydrochloride for (S)-1-cyclopropylethanamine and 4-fluorobenzaldehyde for benzaldehyde.

### Example 889B

### N-(4-fluorobenzyl)-N-(3-methoxycyclobutyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and N-(4-fluorobenzyl)-3-methoxycyclobutanamine for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆) δ 8.67 (d, J=3.1 Hz, 1H), 7.54 (d, J=2.0 Hz, 1H), 7.39-7.04 (m, 6H), 6.18-5.93 (m, 1H), 4.84-4.52 (m, 3H), 4.46-4.06 (m, 1H), 3.18-2.91 (m, 4H), 2.62 (dd, J=4.6, 2.7 Hz, 4H), 2.57-2.34 (m, 2H), 2.26 (d, J=9.3 Hz, 2H), 2.17-1.75 (m, 1H). LC-MS (ESI): 525 (M+H)⁺.

### Example 890

### 3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]-N-methylcyclobutanecarboxamide

### Example 890A

### tert-butyl 3-((4-fluorobenzyl)amino)cyclobutanecarboxylate

The title compound was prepared as described in EXAMPLE 283A, substituting tert-butyl 3-aminocyclobutanecarboxylate for (S)-1-cyclopropylethanamine and 4-fluorobenzaldehyde for benzaldehyde.

### Example 890B

### tert-butyl 3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclobutanecarboxylate

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and tert-butyl 3-((4-fluorobenzyl)amino)cyclobutanecarboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 890C

### (R)-3-(N-(4-fluorobenzyl)-2-(5-(3-methylureido)-2′,4′-dioxo-2,3-dihydrospiro[indene-1,5′-oxazolidin]-3′-yl)acetamido)cyclobutanecarboxylic acid

The title compound was prepared as described in EXAMPLE 302, tert-butyl 3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclobutanecarboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 890D

### 3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]-N-methylcyclobutanecarboxamide

The title compound was prepared as described in EXAMPLE 306, substituting (R)-3-(N-(4-fluorobenzyl)-2-(5-(3-methylureido)-2′,4′-dioxo-2,3-dihydrospiro[indene-1,5′-oxazolidin]-3′-yl)acetamido)cyclobutanecarboxylic acid for cyclopropanecarboxylic acid and methylamine hydrochloride for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆) δ 8.67 (d, J=4.2 Hz, 1H), 7.78-7.63 (m, 1H), 7.51 (dd, J=11.0, 1.8 Hz, 1H), 7.36-6.92 (m, 5H), 6.14-5.94 (m, 1H), 4.89-4.19 (m, 4H), 3.19-2.89 (m, 2H), 2.60 (dd, J=4.7, 3.0 Hz, 4H), 2.56-2.37 (m, 4H), 2.35-2.03 (m, 2H). LC-MS (ESI): 552 (M+H)⁺.

### Example 891

### 3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclobutanecarboxamide

The title compound was prepared as described in EXAMPLE 306, substituting (R)-3-(N-(4-fluorobenzyl)-2-(5-(3-methylureido)-2′,4′-dioxo-2,3-dihydrospiro[indene-1,5′-oxazolidin]-3′-yl)acetamido)cyclobutanecarboxylic acid for cyclopropanecarboxylic acid and ammonia for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (501 MHz, DMSO-d₆) δ 8.69 (d, J=4.6 Hz, 1H), 7.95 (s, 1H), 7.62-7.47 (m, 1H), 7.36-7.03 (m, 5H), 6.77 (d, J=16.6 Hz, 1H), 6.07 (t, J=4.6 Hz, 1H), 4.85-4.19 (m, 4H), 3.21-2.93 (m, 2H), 2.88 (s, 3H), 2.66-2.56 (m, 2H), 2.55-2.41 (m, 2H), 2.24 (dddd, J=53.6, 34.1, 16.3, 8.5 Hz, 4H). LC-MS (ESI): 538 (M+H)⁺.

### Example 892

### 2-{(3′R,4S)-5′-[1-(difluoromethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide

The title compound was prepared as described in EXAMPLE 295, substituting 2-[(3′R,4S)-5′-bromo-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-(difluoromethyl)-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.60 (d, J=14.4 Hz, 2H), 8.18 (s, 1H), 7.84-7.02 (m, 8H), 6.13 (ddd, J=57.6, 6.9, 4.7 Hz, 1H), 5.25-5.02 (m, 1H), 4.84-4.16 (m, 4H), 3.06 (ddd, J=14.4, 12.6, 6.9 Hz, 1H), 2.42-2.14 (m, 1H), 1.33 (d, J=7.1 Hz, 3H). MS (ESI⁺) m/z 598 (M+H)⁺.

### Example 893

### 2-(5-amino-4-cyano-2′,4′-dioxo-2,3-dihydro-3′H-spiro[indene-1,5′-[1,3]oxazolidin]-3′-yl)-N-(4-fluorobenzyl)-N-[(2S)-1,1,1-trifluoropropan-2-yl]acetamide

The title compound was prepared as described in EXAMPLE 284D-E, substituting 2-(5-chloro-4-cyano-2′,4′-dioxo-2,3-dihydro-3′H-spiro[indene-,5′-[1,3]oxazolidin]-3′-yl)-N-(4-fluorobenzyl)-N-[(2S)-1,1,1-trifluoropropan-2-yl]acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide. ¹H NMR (500 MHz, DMSO-d₆) δ 7.43-7.18 (m, 4.3H), 7.12 (td, J=8.7, 5.8 Hz, 0.7H), 6.70 (dd, J=14.3, 6.7 Hz, 1H), 6.51 (s, 2H), 5.39 (s, 0.7H), 5.15-4.67 (m, 2.3H), 4.63-4.36 (m, 1.3H), 4.18 (dd, J=17.1, 4.0 Hz, 0.7H), 3.23-2.93 (m, 2H), 2.75-2.42 (m, 2H), 1.42-1.28 (m, 3H). MS (ESI⁺) m/z 527 (M+Na)⁺.

### Example 894

### 2-[2,5-dioxo-4′-(piperidin-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide

2-[2,5-Dioxo-4′-(1,2,3,6-tetrahydropyridin-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide (50 mg, 0.092 mmol) and methanol (4 ml) were added to 20% palladium(II) hydroxide on carbon, wet (100 mg, 0.363 mmol) in a 50 ml pressure bottle and stirred for 16 hours at 50 psi hydrogen at room temperature. Filtration and silica gel column chromatography afforded the title compound. ¹H NMR (500 MHz, DMSO-d₆) δ 8.79 (d, J=15.4 Hz, 1H), 7.37 (dt, J=8.8, 4.8 Hz, 1.3H), 7.22 (dq, J=19.6, 7.6 Hz, 3H), 7.16-7.06 (m, 2H), 5.41-5.27 (m, 0.7H), 5.10-5.02 (m, 0.3H), 4.93-4.65 (m, 2H), 4.40 (dd, J=16.8, 5.0 Hz, 1.3H), 4.07 (d, J=17.0 Hz, 0.7H), 3.15-2.96 (m, 4H), 2.71 (dtd, J=20.6, 11.8, 3.1 Hz, 2H), 2.59-2.50 (m, 1H), 2.26-2.12 (m, 1H), 2.07-1.93 (m, 1H), 1.75-1.51 (m, 4H), 1.38-1.25 (m, 3H). MS (ESI⁺) 547 (M+H)⁺.

### Example 896

### 4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]benzoic acid

The title compound was prepared as described in EXAMPLE 302, substituting tert-butyl 4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]benzoate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆, 120° C.) δ ppm 8.34-8.20 (m, 1H), 8.01-7.92 (m, 2H), 7.47 (s, 1H), 7.42-7.33 (m, 2H), 7.29-7.17 (m, 4H), 7.10-6.97 (m, 2H), 5.84 (brs, 1H), 4.94 (s, 2H), 4.24-4.07 (m, 2H), 3.17-2.93 (m, 2H), 2.69-2.57 (m, 4H), 2.45-2.36 (m, 1H). MS (ESI⁺) m/z 559 (M+H)⁺.

### Example 898

### 3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]benzoic acid

The title compound was prepared as described in EXAMPLE 302, substituting tert-butyl 3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]benzoate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ ppm 13.21 (brs, 1H), 8.67 (s, 1H), 7.99-7.85 (m, 1H), 7.82-7.72 (m, 1H), 7.60-7.46 (m, 3H), 7.28-7.15 (m, 4H), 7.10 (t, J=8.7 Hz, 2H), 6.15-5.97 (m, 1H), 5.00-4.81 (m, 2H), 4.14-3.95 (m, 2H), 3.14-3.03 (m, 1H), 3.01-2.89 (m, 1H), 2.68-2.54 (m, 4H), 2.46-2.37 (m, 1H). MS (ESI⁺) m/z 559 (M+H)⁺.

### Example 899

### {4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]phenyl}acetic acid

The title compound was prepared as described in EXAMPLE 302, substituting tert-butyl {4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]phenyl}acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ ppm 12.35 (brs, 1H), 8.66 (s, 1H), 7.56-7.47 (m, 1H), 7.37-7.02 (m, 10H), 6.14-5.94 (m, 1H), 4.96-4.78 (m, 2H), 4.13-3.94 (m, 2H), 3.57 (s, 2H), 3.16-3.02 (m, 1H), 3.00-2.88 (m, 1H), 2.68-2.52 (m, 4H), 2.45-2.35 (m, 1H). MS (ESI⁺) m/z 573 (M+H)⁺.

### Example 900

### (2-{[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]methyl}pyrrolidin-1-yl)acetic acid

### Example 900A

### tert-butyl 2-(2-((N-(4-fluorobenzyl)-2-((R)-5-(3-methylureido)-2′,4′-dioxo-2,3-dihydrospiro[indene-1,5′-oxazolidin]-3′-yl)acetamido)methyl)pyrrolidin-1-yl)acetate

A mixture of N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-(pyrrolidin-2-ylmethyl)acetamide (0.047 g, 0.090 mmol) and triethylamine (0.038 ml, 0.269 mmol) in N,N-dimethylformamide (0.898 ml) was treated with tert-butyl 2-bromoacetate (0.016 ml, 0.108 mmol) and the reaction mixture was stirred at room temperature for 30 hours. The reaction mixture was partitioned between saturated aqueous sodium bicarbonate and ethyl acetate. The organic layer was washed with water and brine, dried over anhydrous sodium sulfate, filtered, and concentrated. The residue was purified by silica gel column chromatography to afford the title compound.

### Example 900B

### (2-{[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]methyl}pyrrolidin-1-yl)acetic acid

The title compound was prepared as described in EXAMPLE 302, substituting tert-butyl 2-(2-((N-(4-fluorobenzyl)-2-((R)-5-(3-methylureido)-2′,4′-dioxo-2,3-dihydrospiro[indene-1,5′-oxazolidin]-3′-yl)acetamido)methyl)pyrrolidin-1-yl)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ ppm 9.80 (brs, 1H), 8.86-8.70 (m, 1H), 7.57-7.42 (m, 1H), 7.39-7.11 (m, 6H), 6.30-6.08 (m, 1H), 4.82-4.42 (m, 4H), 4.35-3.46 (m, 7H), 3.29-2.88 (m, 3H), 2.69-2.54 (m, 3H), 2.48-2.38 (m, 1H), 2.20-1.62 (m, 4H). MS (ESI⁺) m/z 582 (M+H)⁺.

### Example 902

### N-(4-fluorobenzyl)-2-[(4S)-7′-hydroxy-2,5-dioxo-2′,3′-dihydro-1H-spiro[imidazolidine-4,1′-inden]-1-yl]-N-[(2 S)-1,1,1-trifluoropropan-2-yl]acetamide

### Example 902A

### 2-((S)-2,5-dioxo-7′-(4,4,5,5-tetramethyl-1,3,2-dioxaborolan-2-yl)-2′,3′-dihydrospiro[imidazolidine-4,1′-inden]-1-yl)-N-(4-fluorobenzyl)-N—((S)-1,1,1-trifluoropropan-2-yl)acetamide

The title compound was prepared as described in EXAMPLE 353A, substituting 2-[(4S)-7′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-((R)-5-bromo-2′,4′-dioxo-2,3-dihydrospiro[indene-1,5′-oxazolidin]-3′-yl)-N—((S)-1-cyclopropylethyl)-N-(4-fluorobenzyl)acetamide.

### Example 902B

### N-(4-fluorobenzyl)-2-[(4S)-7′-hydroxy-2,5-dioxo-2′,3′-dihydro-1H-spiro[imidazolidine-4,1′-inden]-1-yl]-N-[(2 S)-1,1,1-trifluoropropan-2-yl]acetamide

A solution of 2-((S)-2,5-dioxo-7′-(4,4,5,5-tetramethyl-1,3,2-dioxaborolan-2-yl)-2′,3′-dihydrospiro[imidazolidine-4,1′-inden]-1-yl)-N-(4-fluorobenzyl)-N—((S)-1,1,1-trifluoropropan-2-yl)acetamide (0.02 g, 0.034 mmol) in methanol (0.3 mL) was treated with hydrogen peroxide (8.85 mg, 0.078 mmol) (30% in water) and the reaction mixture was stirred at room temperature for 20 hours. The reaction mixture was directly purified by preparative reverse-phase HPLC to afford the title compound. ¹H NMR (400 MHz, DMSO-d₆, 120° C.) δ ppm 9.00 (brs, 1H), 8.07 (brs, 1H), 7.42-7.26 (m, 2H), 7.18-7.01 (m, 3H), 6.69 (d, J=7.5 Hz, 1H), 6.57 (d, J=8.0 Hz, 1H), 5.26-5.06 (m, 1H), 4.87-4.51 (m, 2H), 4.48-4.15 (m, 2H), 3.13-2.97 (m, 1H), 2.97-2.89 (m, 1H), 2.60-2.48 (m, 1H), 2.22-2.10 (m, 1H), 1.33 (d, J=7.1 Hz, 3H). MS (APCI⁺) m/z 480 (M+H)⁺.

### Example 903

### {3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]phenyl}acetic acid

A solution of methyl {3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]phenyl}acetate (0.05 g, 0.085 mmol) in 1,2-dichloroethane (1.416 ml) was treated with trimethylstannanol (0.123 g, 0.680 mmol) and the reaction mixture was heated at 75° C. for 16 hours. The reaction mixture was cooled to room temperature and concentrated. The residue was taken up in ethyl acetate and washed with aqueous 1N HCl (twice) and brine. The organic layer was dried over anhydrous sodium sulfate, filtered, and concentrated. The concentrate was purified by preparative reverse-phase HPLC to give the title compound. ¹H NMR (400 MHz, DMSO-d₆) δ ppm 12.36 (brs, 1H), 8.69 (s, 1H), 7.59-7.51 (m, 1H), 7.40 (t, J=7.8 Hz, 1H), 7.33-7.15 (m, 6H), 7.16-7.07 (m, 2H), 6.17-6.00 (m, 1H), 5.75 (s, 1H), 4.97-4.84 (m, 2H), 4.15-3.99 (m, 2H), 3.60 (s, 2H), 3.18-3.06 (m, 1H), 3.05-2.92 (m, 1H), 2.68-2.57 (m, 4H), 2.48-2.40 (m, 1H). MS (ESI⁺) m/z 573 (M−H)⁺.

### Example 904

### tert-butyl {4-[{[(1R)-5-bromo-2′,4′-dioxo-2,3-dihydro-3′H-spiro[indene-1,5′-[1,3]oxazolidin]-3′-yl]acetyl}(4-fluorobenzyl)amino]cyclohexylidene}acetate

### Example 904A

### tert-butyl 2-(4-(2-bromo-N-(4-fluorobenzyl)acetamido)cyclohexylidene)acetate

The title compound was prepared as described in EXAMPLE 281B, substituting tert-butyl 2-(4-((4-fluorobenzyl)amino)cyclohexylidene)acetate for (S)-1,1,1-trifluoro-N-(4-fluorobenzyl)propan-2-amine.

### Example 904B

### tert-butyl {4-[{[(1R)-5-bromo-2′,4′-dioxo-2,3-dihydro-3′H-spiro[indene-1,5′-[1,3]oxazolidin]-3′-yl]acetyl}(4-fluorobenzyl)amino]cyclohexylidene}acetate

The title compound was prepared as described in EXAMPLE 281F, substituting (R)-5-bromo-2,3-dihydrospiro[indene-1,5′-oxazolidine]-2′,4′-dione for (R)-5-bromo-2,3-dihydrospiro[indene-1,5′-oxazolidine]-2′,4′-dione and tert-butyl 2-(4-(2-bromo-N-(4-fluorobenzyl)acetamido)cyclohexylidene)acetate for (S)-2-bromo-N-(4-fluorobenzyl)-N-(1,1,1-trifluoropropan-2-yl)acetamide. ¹H NMR (400 MHz, methanol-d₄) δ 7.57 (s, 1H), 7.49-7.41 (m, 2H), 7.36 (dd, J=8.4, 5.2 Hz, 1H), 7.24-7.18 (m, 1H), 7.13-7.07 (m, 1H), 7.02-6.94 (m, 1H), 5.53 (d, J=12.2 Hz, 1H), 4.75 (d, J=5.8 Hz, 1H), 4.68-4.49 (m, 2H), 4.49-4.35 (m, 1H), 3.93-3.71 (m, 1H), 3.27-3.00 (m, 2H), 2.77 (tdd, J=14.7, 8.6, 6.6 Hz, 1H), 2.56 (dddd, J=14.5, 12.5, 8.3, 4.3 Hz, 1H), 2.39-2.14 (m, 3H), 1.91 (td, J=30.4, 27.2, 8.7 Hz, 3H), 1.72-1.47 (m, 2H), 1.42 (d, J=4.1 Hz, 9H). MS (ESI⁺) m/z 642 (M+H)⁺.

### Example 905

### tert-butyl {4-[{[(4S)-5′-bromo-2,5-dioxo-2′,3′-dihydro-1H-spiro[imidazolidine-4,1′-inden]-1-yl]acetyl}(4-fluorobenzyl)amino]cyclohexylidene}acetate

The title compound was prepared as described in EXAMPLE 281F, substituting (S)-5′-bromo-2′,3′-dihydrospiro[imidazolidine-4,1′-indene]-2,5-dione for (R)-5-bromo-2,3-dihydrospiro[indene-1,5′-oxazolidine]-2′,4′-dione and tert-butyl 2-(4-(2-bromo-N-(4-fluorobenzyl)acetamido)cyclohexylidene)acetate for (S)-2-bromo-N-(4-fluorobenzyl)-N-(1,1,1-trifluoropropan-2-yl)acetamide. ¹H NMR (400 MHz, methanol-d₄) δ 7.48 (d, J=1.7 Hz, 1H), 7.43-7.32 (m, 2H), 7.29 (dd, J=8.2, 4.9 Hz, 1H), 7.20 (dd, J=8.5, 5.4 Hz, 1H), 7.13-7.05 (m, 1H), 6.96 (t, J=8.7 Hz, 1H), 5.51 (d, J=12.1 Hz, 1H), 4.67-4.44 (m, 3H), 4.31 (t, J=2.0 Hz, 1H), 4.10 (dq, J=18.2, 7.6, 7.1 Hz, 1H), 3.81 (t, J=13.0 Hz, 1H), 3.21-2.98 (m, 2H), 2.79-2.58 (m, 1H), 2.41-2.17 (m, 3H), 1.99-1.74 (m, 3H), 1.56 (tt, J=16.7, 7.7 Hz, 2H), 1.42 (d, J=4.0 Hz, 9H). MS (ESI⁺) m/z 641 (M+H)⁺.

### Example 906

### tert-butyl {4-[(4-fluorobenzyl) {[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]cyclohexylidene}acetate

The title compound was prepared as described in EXAMPLE 295, substituting tert-butyl {4-[{[(1R)-5-bromo-2′,4′-dioxo-2,3-dihydro-3′H-spiro[indene-1,5′-[1,3]oxazolidin]-3′-yl]acetyl}(4-fluorobenzyl)amino]cyclohexylidene}acetate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, methanol-d₄) δ 7.98 (d, J=5.7 Hz, 1H), 7.84 (d, J=4.7 Hz, 1H), 7.56 (s, 1H), 7.50 (dd, J=11.7, 3.7 Hz, 2H), 7.38 (dd, J=8.5, 5.2 Hz, 1H), 7.23 (dd, J=8.5, 5.3 Hz, 1H), 7.12 (t, J=8.7 Hz, 1H), 7.00 (t, J=8.7 Hz, 1H), 5.54 (d, J=12.2 Hz, 1H), 4.77 (d, J=7.0 Hz, 1H), 4.68-4.51 (m, 2H), 4.51-4.36 (m, 1H), 4.11 (dd, J=13.6, 6.5 Hz, 1H), 3.91 (d, J=3.5 Hz, 3H), 3.84 (t, J=13.9 Hz, 1H), 3.23 (q, J=6.9 Hz, 1H), 3.14 (tt, J=11.2, 4.6 Hz, 1H), 2.89-2.72 (m, 1H), 2.67-2.49 (m, 1H), 2.45-2.20 (m, 2H), 1.89 (dd, J=28.0, 13.0 Hz, 3H), 1.60 (dt, J=20.8, 13.3 Hz, 2H), 1.44 (d, J=4.0 Hz, 9H). MS (ESI⁺) m/z 643 (M+H)⁺.

### Example 907

### tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate

The title compound was prepared as described in EXAMPLE 295, substituting tert-butyl {4-[{[(4S)-5′-bromo-2,5-dioxo-2′,3′-dihydro-1H-spiro[imidazolidine-4,1′-inden]-1-yl]acetyl}(4-fluorobenzyl)amino]cyclohexylidene}acetate and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, Methanol-d₄) δ 7.92 (d, J=3.4 Hz, 1H), 7.78 (d, J=2.5 Hz, 1H), 7.48 (s, 1H), 7.46-7.30 (m, 3H), 7.21 (dd, J=8.4, 5.3 Hz, 1H), 7.09 (t, J=8.7 Hz, 1H), 6.96 (t, J=8.7 Hz, 1H), 5.52 (d, J=12.1 Hz, 1H), 4.70-4.47 (m, 3H), 4.32 (t, J=2.5 Hz, 1H), 4.13 (t, J=12.0 Hz, 1H), 3.89 (d, J=2.0 Hz, 3H), 3.81 (t, J=13.6 Hz, 1H), 3.23-3.00 (m, 2H), 2.72 (dddd, J=13.1, 11.2, 8.0, 5.0 Hz, 1H), 2.41-2.17 (m, 3H), 2.07-1.72 (m, 3H), 1.70-1.46 (m, 2H), 1.42 (d, J=3.8 Hz, 9H). MS (ESI⁺) m/z 642 (M+H)⁺.

### Example 908

### {4-[(4-fluorobenzyl) {[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]cyclohexylidene}acetic acid

The title compound was prepared as described in EXAMPLE 302, substituting tert-butyl {4-[(4-fluorobenzyl) {[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]cyclohexylidene}acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, methanol-d₄) δ 7.98 (d, J=5.8 Hz, 1H), 7.84 (d, J=4.8 Hz, 1H), 7.56 (s, 1H), 7.49 (dd, J=12.3, 4.8 Hz, 2H), 7.38 (dd, J=8.5, 5.2 Hz, 1H), 7.23 (dd, J=8.5, 5.3 Hz, 1H), 7.12 (t, J=8.7 Hz, 1H), 6.99 (t, J=8.7 Hz, 1H), 5.62 (d, J=11.8 Hz, 1H), 4.80-4.73 (m, 1H), 4.68-4.52 (m, 2H), 4.51-4.37 (m, 1H), 4.14 (td, J=11.6, 5.8 Hz, 1H), 3.91 (d, J=3.4 Hz, 3H), 3.86 (d, J=13.6 Hz, 1H), 3.24 (dq, J=13.0, 6.8, 5.9 Hz, 1H), 3.14 (ddd, J=11.6, 9.2, 4.5 Hz, 1H), 2.79 (tdd, J=14.5, 8.6, 6.7 Hz, 1H), 2.66-2.49 (m, 1H), 2.34 (d, J=17.1 Hz, 2H), 2.14-1.76 (m, 3H), 1.61 (qd, J=14.8, 12.7, 6.8 Hz, 2H). MS (ESI⁺) m/z 587 (M+H)⁺.

### Example 909

### {4-[{[(4S)-5′-amino-2,5-dioxo-2′,3′-dihydro-1H-spiro[imidazolidine-4,1′-inden]-1-yl]acetyl}(4-fluorobenzyl)amino]cyclohexylidene}acetic acid

### Example 909A

### tert-butyl 2-(4-(2-((S)-5′-amino-2,5-dioxo-2′,3′-dihydrospiro[imidazolidine-4,1′-inden]-1-yl)-N-(4-fluorobenzyl)acetamido)cyclohexylidene)acetate

The title compound was prepared as described in EXAMPLE 284D-E, substituting tert-butyl {4-[{[(4S)-5′-bromo-2,5-dioxo-2′,3′-dihydro-1H-spiro[imidazolidine-4,1′-inden]-1-yl]acetyl}(4-fluorobenzyl)amino]cyclohexylidene}acetate for N-benzyl-2-((S)-5-bromo-2′,4′-dioxo-2,3-dihydrospiro[indene-1,5′-oxazolidin]-3′-yl)-N—((S)-1-cyclopropylethyl)acetamide.

### Example 909B

### {4-[{[(4S)-5′-amino-2,5-dioxo-2′,3′-dihydro-1H-spiro[imidazolidine-4,1′-inden]-1-yl]acetyl}(4-fluorobenzyl)amino]cyclohexylidene}acetic acid

The title compound was prepared as described in EXAMPLE 302, substituting tert-butyl 2-(4-(2-((S)-5′-amino-2,5-dioxo-2′,3′-dihydrospiro[imidazolidine-4,1′-inden]-1-yl)-N-(4-fluorobenzyl)acetamido)cyclohexylidene)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5-[1,3]oxazolidin]-5-yl]azetidine-1-carboxylate. ¹H NMR (400 MHz, methanol-d₄) δ 7.52-7.42 (m, 1H), 7.38 (dd, J=8.5, 5.1 Hz, 1H), 7.26-7.17 (m, 1H), 7.16-7.07 (m, 1H), 7.05-6.90 (m, 3H), 5.63 (d, J=11.9 Hz, 1H), 4.76 (d, J=7.1 Hz, 1H), 4.72-4.51 (m, 2H), 4.50-4.38 (m, 1H), 4.14 (ddt, J=12.0, 7.8, 3.7 Hz, 1H), 3.87 (t, J=13.1 Hz, 1H), 3.20 (dq, J=14.0, 6.9 Hz, 1H), 3.14-3.00 (m, 1H), 2.78 (tdd, J=14.8, 8.7, 6.8 Hz, 1H), 2.65-2.48 (m, 1H), 2.47-2.22 (m, 2H), 2.11-1.79 (m, 3H), 1.75-1.48 (m, 2H). MS (ESI⁺) m/z 521 (M+H)⁺.

### Example 910

### N-(4-fluorobenzyl)-2-[(1 S,3R)-3-fluoro-5-(1-methyl-1H-pyrazol-4-yl)-2′,4′-dioxo-2,3-dihydro-3′H-spiro[indene-1,5′-[1,3]oxazolidin]-3′-yl]-N-[(2S)-1,1,1-trifluoropropan-2-yl]acetamide

### Example 910A

### 2-((1 S,3R)-5-bromo-3-fluoro-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide

The title compound was prepared as described in EXAMPLE 887, substituting 2-[(1S,3S)-5-bromo-3-hydroxy-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for N-(4-fluorobenzyl)-2-[(1S,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide.

### Example 910B

### N-(4-fluorobenzyl)-2-[(1S,3R)-3-fluoro-5-(1-methyl-1H-pyrazol-4-yl)-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-((1S,3R)-5-bromo-3-fluoro-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.09 (s, 1H), 7.83 (s, 1H), 7.80-7.62 (m, 2H), 7.51 (d, J=8.1 Hz, 1H), 7.33 (dd, J=8.4, 5.4 Hz, 2H), 7.12 (t, J=8.7 Hz, 2H), 6.11 (ddd, J=56.3, 6.4, 2.4 Hz, 1H), 5.2 (m, 1H). 4.91-4.24 (m, 4H), 3.15 (ddd, J=20.7, 15.8, 6.4 Hz, 1H), 2.65 (ddd, J=21.7, 15.8, 2.4 Hz, 1H), 1.37 (d, J=7.1 Hz, 3H). MS (ESI⁺) m/z 563 (M+H)⁺.

### Example 911

### 2-[(4S)-5′-bromo-6′-fluoro-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 281F, substituting (S)-5′-bromo-6′-fluorospiro[imidazolidine-4,1′-indene]-2,3′,5(2′H)-trione for (R)-5-bromo-2,3-dihydrospiro[indene-1,5′-oxazolidine]-2′,4′-dione. ¹H NMR (400 MHz, DMSO-d₆) δ 8.97 (d, J=7.8 Hz, 1H), 8.12 (d, J=6.3 Hz, 1H), 7.68 (t, J=8.3 Hz, 1H), 7.36 (dd, J=8.4, 5.3 Hz, 1.3H), 7.26-7.15 (m, 2H), 7.08 (t, J=8.7 Hz, 0.7H), 5.41-5.28 (m, 0.7H), 5.09-4.98 (m, 0.3H), 4.94-4.65 (m, 2H), 4.48-4.36 (m, 1.3H), 4.13 (d, J=17.0 Hz, 0.7H), 3.20-2.94 (m, 2H), 1.32 (dd, J=25.1, 6.9 Hz, 3H). MS (ESI⁺) m/z 574 (M+H)⁺.

### Example 912

### 2-[(3′R,4R)-5′-bromo-6′-fluoro-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 570A, substituting 2-[(4S)-5′-bromo-6′-fluoro-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide. ¹H NMR (400 MHz, DMSO-d₆, 120° C.) δ 8.44 (s, 1H), 7.66 (d, J=6.5 Hz, 1H), 7.38-7.29 (m, 2H), 7.23 (d, J=8.5 Hz, 1H), 7.11 (t, J=8.8 Hz, 2H), 5.29 (q, J=6.5 Hz, 1H), 5.17 (s, 2H), 4.81 (d, J=17.6 Hz, 1H), 4.59 (d, J=17.2 Hz, 1H), 4.48 (d, J=16.4 Hz, 1H), 4.26 (d, J=16.7 Hz, 1H), 2.54 (dd, J=13.6, 6.8 Hz, 1H), 2.38 (dd, J=13.6, 6.4 Hz, 1H), 1.34 (d, J=7.1 Hz, 3H). MS (ESI⁺) m/z 558 (M−H₂O+H)⁺.

### Example 913

### 2-[(3′S,4S)-5′-bromo-6′-fluoro-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 570A, substituting 2-[(4S)-5′-bromo-6′-fluoro-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide. ¹H NMR (400 MHz, DMSO-d₆, 120° C.) δ 8.45 (s, 1H), 7.65 (d, J=6.6 Hz, 1H), 7.33 (dd, J=8.4, 5.4 Hz, 2H), 7.22 (d, J=8.5 Hz, 1H), 7.10 (t, J=8.7 Hz, 2H), 5.29 (q, J=6.5 Hz, 1H), 5.16 (d, J=7.8 Hz, 2H), 4.80 (d, J=17.5 Hz, 1H), 4.60 (d, J=17.6 Hz, 1H), 4.47 (d, J=16.7 Hz, 1H), 4.27 (d, J=16.6 Hz, 1H), 2.54 (dd, J=13.7, 6.9 Hz, 1H), 2.39 (dd, J=13.6, 6.4 Hz, 1H), 1.34 (d, J=7.0 Hz, 3H). MS (ESI⁻) m/z 574 (M−H)⁻.

### Example 914

### N-(4-fluorobenzyl)-2-[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]-N-(tetrahydro-2H-pyran-4-yl)acetamide

### Example 914A

### (S)-tert-butyl 2-(5′-bromo-2,3′,5-trioxo-2′,3′-dihydrospiro[imidazolidine-4,1′-inden]-1-yl)acetate

The title compound was prepared as described in EXAMPLE 832A, substituting (S)-5′-bromospiro[imidazolidine-4,1′-indene]-2,3′,5 (2′H)-trione for 5′-bromo-2′,3′-dihydrospiro[imidazolidine-4,1′-indene]-2,5-dione.

### Example 914B

### tert-butyl 2-((1′S,3′S)-5′-bromo-3′-hydroxy-2,5-dioxo-2′,3′-dihydrospiro[imidazolidine-4,1′-inden]-1-yl)acetate

The title compound was prepared as described in EXAMPLE 570A, substituting (S)-tert-butyl 2-(5′-bromo-2,3′,5-trioxo-2′,3′-dihydrospiro[imidazolidine-4,1′-inden]-1-yl)acetate for 2-(5′-bromo-2,3′,5-trioxo-2′,3′-dihydrospiro[imidazolidine-4,1′-inden]-1-yl)-N-(4-fluorobenzyl)-N—((S)-1,1,1-trifluoropropan-2-yl)acetamide.

### Example 914C

### tert-butyl 2-((1′S,3′R)-5′-bromo-3′-fluoro-2,5-dioxo-2′,3′-dihydrospiro[imidazolidine-4,1′-inden]-1-yl)acetate

The title compound was prepared as described in EXAMPLE 887, substituting tert-butyl 2-((1′S,3′S)-5′-bromo-3′-hydroxy-2,5-dioxo-2′,3′-dihydrospiro[imidazolidine-4,1′-inden]-1-yl)acetate for N-(4-fluorobenzyl)-2-[(1S,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide.

### Example 914D

### tert-butyl 2-((1′S,3′R)-3′-fluoro-5′-(1-methyl-1H-pyrazol-4-yl)-2,5-dioxo-2′,3′-dihydrospiro[imidazolidine-4,1′-inden]-1-yl)acetate

The title compound was prepared as described in EXAMPLE 295, substituting tert-butyl 2-((1′S,3′R)-5′-bromo-3′-fluoro-2,5-dioxo-2′,3′-dihydrospiro[imidazolidine-4,1′-inden]-1-yl)acetate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol.

### Example 914E

### 2-((1′S,3′R)-3′-fluoro-5′-(1-methyl-1H-pyrazol-4-yl)-2,5-dioxo-2′,3′-dihydrospiro[imidazolidine-4,1′-inden]-1-yl)acetic acid

The title compound was prepared as described in EXAMPLE 302, substituting tert-butyl 2-((1′S,3′R)-3′-fluoro-5′-(1-methyl-1H-pyrazol-4-yl)-2,5-dioxo-2′,3′-dihydrospiro[imidazolidine-4, 1′-inden]-1-yl)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 914F

### N-(4-fluorobenzyl)tetrahydro-2H-pyran-4-amine

The title compound was prepared as described in EXAMPLE 283A, substituting tetrahydro-2H-pyran-4-amine for (S)-1-cyclopropylethanamine and 4-fluorobenzaldehyde for benzaldehyde.

### Example 914G

### N-(4-fluorobenzyl)-2-[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]-N-(tetrahydro-2H-pyran-4-yl)acetamide

The title compound was prepared as described in EXAMPLE 306, substituting 2-((1′S,3′R)-3′-fluoro-5′-(1-methyl-1H-pyrazol-4-yl)-2,5-dioxo-2′,3′-dihydrospiro[imidazolidine-4,1′-inden]-1-yl)acetic acid and N-(4-fluorobenzyl)tetrahydro-2H-pyran-4-amine for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆) δ 9.00 (d, J=8.8 Hz, 2H), 8.21 (d, J=5.9 Hz, 2H), 7.92 (d, J=6.1 Hz, 2H), 7.80-7.66 (m, 4H), 7.48-7.32 (m, 6H), 7.32-7.06 (m, 8H), 6.21 (q, J=5.4 Hz, 1H), 4.64 (s, 2H), 4.54 (d, J=10.9 Hz, 5H), 4.21 (s, 3H), 3.91-3.59 (m, 14H), 3.36 (t, J=11.7 Hz, 4H), 3.25-3.18 (m, 3H), 3.05 (tt, J=12.8, 6.2 Hz, 2H), 2.79 (d, J=63.4 Hz, 4H), 2.35 (dtd, J=25.8, 14.1, 4.3 Hz, 3H), 1.80-1.00 (m, 15H), 0.83 (d, J=6.4 Hz, 1H). MS (ESI⁺) m/z 550 (M+H)⁺.

### Example 915

### 2-(2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide

The title compound was prepared as described in EXAMPLE 281F, substituting 2′,3′-dihydrospiro[imidazolidine-4,1′-indene]-2,5-dione for (R)-5-bromo-2,3-dihydrospiro[indene-1,5′-oxazolidine]-2′,4′-dione. ¹H NMR (400 MHz, DMSO-d₆) δ 8.35 (s, 1H), 7.40-7.03 (m, 6H), 5.16 (p, J=7.9 Hz, 1H), 4.88-3.94 (m, 4H), 3.04 (t, J=7.2 Hz, 2H), 2.56 (ddd, J=14.4, 7.3, 6.2 Hz, 1H), 2.23 (dtd, J=13.3, 7.7, 1.9 Hz, 1H), 1.33 (d, J=7.1 Hz, 3H). MS (ESI⁺) m/z 464 (M+H)⁺.

### Example 916

### N-(4-fluorobenzyl)-2-{6′-fluoro-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide

The title compound was prepared as described in EXAMPLE 770B, substituting 2-(5′-bromo-6′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide for 2-(7-bromo-2′,4′-dioxospiro[isochroman-4,5′-oxazolidin]-3′-yl)-N-(4-fluorobenzyl)-N—((S)-1,1,1-trifluoropropan-2-yl)acetamide. ¹H NMR (400 MHz, DMSO-d₆) δ 8.75 (s, 1H), 8.29 (t, J=2.7 Hz, 1H), 8.03 (d, J=7.4 Hz, 1H), 7.35 (ddd, J=8.1, 5.1, 2.1 Hz, 1.3H), 7.24-7.13 (m, 2H), 7.13-6.94 (m, 1.7H), 6.45 (d, J=5.1 Hz, 1H), 5.34 (m, 0.7H), 5.11-4.97 (m, 0.3H), 4.92-4.62 (m, 2H), 4.43-4.27 (m, 1.3H), 4.10-3.94 (m, 0.7H), 2.99-2.86 (m, 2H), 2.62 (d, J=4.6 Hz, 3H), 2.56-2.48 (m, 1H), 2.25-2.11 (m, 1H), 1.29 (dd, J=24.9, 5.7 Hz, 3H). MS (ESI⁺) m/z 554 (M+H)⁺.

### Example 917

### 2-{(3′R,4S)-3′,6′-difluoro-5′-[(methylcarbamoyl)amino]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide

The title compound was prepared as described in EXAMPLE 770B, substituting 2-((1′S,3′R)-5′-bromo-3′,6′-difluoro-2,5-dioxo-2′,3′-dihydrospiro[imidazolidine-4,1′-inden]-1-yl)-N-(4-fluorobenzyl)-N—((S)-1,1,1-trifluoropropan-2-yl)acetamide for 2-(7-bromo-2′,4′-dioxospiro[isochroman-4,5′-oxazolidin]-3′-yl)-N-(4-fluorobenzyl)-N—((S)-1,1,1-trifluoropropan-2-yl)acetamide. ¹H NMR (400 MHz, DMSO-d₆, 120° C.) δ 8.56 (s, 1H), 8.25 (d, J=7.3 Hz, 1H), 8.09 (s, 1H), 7.31 (dd, J=8.5, 5.4 Hz, 2H), 7.09 (dd, J=9.7, 7.9 Hz, 3H), 6.37 (s, 1H), 6.01 (ddd, J=57.9, 6.8, 4.2 Hz, 1H), 5.28-5.03 (m, 1H), 4.78 (d, J=17.5 Hz, 1H), 4.58 (d, J=17.7 Hz, 1H), 4.43 (d, J=16.6 Hz, 1H), 4.24 (s, 1H), 3.02 (td, J=14.1, 6.8 Hz, 1H), 2.36 (ddd, J=25.2, 14.5, 4.2 Hz, 1H), 1.33 (d, J=7.1 Hz, 3H). MS (ESI⁺) 572 (M+H)⁺.

### Example 918

### N-(azetidin-3-ylmethyl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 894, substituting benzyl 3-{[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]methyl}azetidine-1-carboxylate for 2-[2,5-dioxo-4′-(1,2,3,6-tetrahydropyridin-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide. ¹H NMR (400 MHz, DMSO-d₆, 120° C.) δ ppm 8.28 (brs, 1H), 7.51-7.43 (m, 1H), 7.38-7.19 (m, 4H), 7.11 (t, J=8.7 Hz, 2H), 5.91-5.81 (m, 1H), 4.68-4.41 (m, 4H), 3.64-3.41 (m, 4H), 3.30-2.91 (m, 5H), 2.91-2.83 (m, 1H), 2.73-2.61 (m, 4H), 2.52-2.42 (m, 1H). MS (ESI⁺) m/z 510 (M+H)⁺.

### Example 919

### {4-[{[(1R)-2′,4′-dioxo-2,3-dihydro-3′H-spiro[indene-1,5′-[1,3]oxazolidin]-3′-yl]acetyl}(4-fluorobenzyl)amino]cyclohexylidene}acetic acid

### Example 919A

### tert-butyl 2-(4-(2-((R)-2′,4′-dioxo-2,3-dihydrospiro[indene-1,5′-oxazolidin]-3′-yl)-N-(4-fluorobenzyl)acetamido)cyclohexylidene)acetate

The title compound was prepared as described in EXAMPLE 281F, substituting (R)-2,3-dihydrospiro[indene-1,5′-oxazolidine]-2′,4′-dione for (R)-5-bromo-2,3-dihydrospiro[indene-1,5′-oxazolidine]-2′,4′-dione and tert-butyl 2-(4-(2-bromo-N-(4-fluorobenzyl)acetamido)cyclohexylidene)acetate for (S)-2-bromo-N-(4-fluorobenzyl)-N-(1,1,1-trifluoropropan-2-yl)acetamide.

### Example 919B

### {4-[{[(1R)-2′,4′-dioxo-2,3-dihydro-3′H-spiro[indene-1,5′-[1,3]oxazolidin]-3′-yl]acetyl}(4-fluorobenzyl)amino]cyclohexylidene}acetic acid

The title compound was prepared as described in EXAMPLE 302, substituting tert-butyl 2-(4-(2-((R)-2′,4′-dioxo-2,3-dihydrospiro[indene-1,5′-oxazolidin]-3′-yl)-N-(4-fluorobenzyl)acetamido)cyclohexylidene)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, methanol-d₄) δ 7.51 (dd, J=11.8, 7.7 Hz, 1H), 7.37 (dq, J=11.7, 5.8, 5.3 Hz, 3H), 7.28 (q, J=6.5, 6.1 Hz, 1H), 7.21 (dd, J=8.6, 5.5 Hz, 1H), 7.09 (t, J=8.5 Hz, 1H), 6.96 (q, J=8.9 Hz, 1H), 5.68-5.53 (m, 1H), 4.87-4.70 (m, 2H), 4.67-4.35 (m, 3H), 3.96-3.76 (m, 1H), 3.27-3.02 (m, 2H), 2.75 (ddd, J=14.6, 10.1, 4.5 Hz, 1H), 2.53 (ddd, J=14.8, 8.2, 4.1 Hz, 1H), 2.30 (t, J=11.2 Hz, 2H), 2.07-1.74 (m, 3H), 1.57 (tq, J=16.5, 13.0, 7.6, 3.9 Hz, 2H). MS (ESI⁺) m/z 507 (M+H)⁺.

### Example 920

### methyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate

### Example 920

### methyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate

### Example 920A

### tert-butyl 2-(4-(2-((S)-5′-bromo-2,5-dioxo-2′,3′-dihydrospiro[imidazolidine-4,1′-inden]-1-yl)-N-(4-fluorobenzyl)acetamido)cyclohexylidene)acetate

The title compound was prepared as described in EXAMPLE 281F, substituting (S)-5′-bromo-2′,3′-dihydrospiro[imidazolidine-4,1′-indene]-2,5-dione for (R)-5-bromo-2,3-dihydrospiro[indene-1,5′-oxazolidine]-2′,4′-dione and tert-butyl 2-(4-(2-bromo-N-(4-fluorobenzyl)acetamido)cyclohexylidene)acetate for (S)-2-bromo-N-(4-fluorobenzyl)-N-(1,1,1-trifluoropropan-2-yl)acetamide.

### Example 920B

### tert-butyl 2-(4-(N-(4-fluorobenzyl)-2-((S)-5′-(1-methyl-1H-pyrazol-4-yl)-2,5-dioxo-2′,3′-dihydrospiro[imidazolidine-4,1′-inden]-1-yl)acetamido)cyclohexylidene)acetate

The title compound was prepared as described in EXAMPLE 295, substituting tert-butyl 2-(4-(2-((S)-5′-bromo-2,5-dioxo-2′,3′-dihydrospiro[imidazolidine-4,1′-inden]-1-yl)-N-(4-fluorobenzyl)acetamido)cyclohexylidene)acetate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol.

### Example 920C

### 2-(4-(N-(4-fluorobenzyl)-2-((S)-5′-(1-methyl-1H-pyrazol-4-yl)-2,5-dioxo-2′,3′-dihydrospiro[imidazolidine-4,1′-inden]-1-yl)acetamido)cyclohexylidene)acetic acid

The title compound was prepared as described in EXAMPLE 302, substituting tert-butyl 2-(4-(N-(4-fluorobenzyl)-2-((S)-5′-(1-methyl-1H-pyrazol-4-yl)-2,5-dioxo-2′,3′-dihydrospiro[imidazolidine-4,1′-inden]-1-yl)acetamido)cyclohexylidene)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 920D

### methyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate

To a solution of 2-(4-(N-(4-fluorobenzyl)-2-((S)-5′-(1-methyl-1H-pyrazol-4-yl)-2,5-dioxo-2′,3′-dihydrospiro[imidazolidine-4,1′-inden]-1-yl)acetamido)cyclohexylidene)acetic acid (100 mg, 0.17 mmol) in methanol (5 ml) at 0° C. was added sulfurous dichloride (40.6 mg, 0.34 mmol) dropwise. The mixture was allowed to reach room temperature and was stirred for 3 hours. The volatiles were removed by rotavap and the residue was purified by reverse-phase HPLC (Zorbax RX-C18 column using a gradient of 15% to 100% methanol/0.1% aqueous trifluoroacetic acid) to afford the title compound. ¹H NMR (400 MHz, Methanol-d₄) δ 7.91 (d, J=3.3 Hz, 1H), 7.77 (d, J=2.6 Hz, 1H), 7.51-7.45 (m, 1H), 7.45-7.30 (m, 3H), 7.20 (dd, J=8.5, 5.4 Hz, 1H), 7.08 (t, J=8.7 Hz, 1H), 6.96 (t, J=8.7 Hz, 1H), 5.61 (d, J=11.8 Hz, 1H), 4.70-4.45 (m, 3H), 4.31 (t, J=2.4 Hz, 1H), 4.20-4.02 (m, 1H), 3.88 (d, J=1.8 Hz, 3H), 3.83 (d, J=12.1 Hz, 1H), 3.62 (d, J=4.9 Hz, 3H), 3.13 (tt, J=8.1, 3.6 Hz, 2H), 2.79-2.62 (m, 1H), 2.44-2.18 (m, 3H), 2.07-1.74 (m, 3H), 1.54 (dtd, J=24.9, 12.6, 5.6 Hz, 2H). MS (ESI⁺) m/z 600 (M+H)⁺.

### Example 921

### {4-[(4-fluorobenzyl) {[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid

### Example 921A

### tert-butyl 2-(4-(2-((1′S,3′R)-3′-fluoro-5′-(1-methyl-1H-pyrazol-4-yl)-2,5-dioxo-2′,3′-dihydrospiro[imidazolidine-4,1′-inden]-1-yl)-N-(4-fluorobenzyl)acetamido)cyclohexylidene)acetate

The title compound was prepared as described in EXAMPLE 306, substituting 2-((1′S,3′R)-3′-fluoro-5′-(1-methyl-1H-pyrazol-4-yl)-2,5-dioxo-2′,3′-dihydrospiro[imidazolidine-4,1′-inden]-1-yl)acetic acid for cyclopropanecarboxylic acid and tert-butyl 2-(4-((4-fluorobenzyl)amino)cyclohexylidene)acetate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 921B

### {4-[(4-fluorobenzyl) {[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid

The title compound was prepared as described in EXAMPLE 302, substituting tert-butyl 2-(4-(2-((1′S,3′R)-3′-fluoro-5′-(1-methyl-1H-pyrazol-4-yl)-2,5-dioxo-2′,3′-dihydrospiro[imidazolidine-4,1′-inden]-1-yl)-N-(4-fluorobenzyl)acetamido)cyclohexylidene)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (501 MHz, DMSO-d₆) δ 9.01 (d, J=13.7 Hz, 2H), 8.41 (s, 1H), 8.21 (d, J=8.2 Hz, 2H), 7.93 (dd, J=8.3, 0.8 Hz, 2H), 7.79-7.68 (m, 4H), 7.45-7.04 (m, 1H), 6.21 (td, J=7.1, 4.4 Hz, 1H), 6.09 (td, J=7.0, 4.3 Hz, 1H), 5.55 (d, J=8.5 Hz, 2H), 5.17-4.31 (m, 1H), 4.13 (s, 5H), 3.85 (d, J=4.2 Hz, 6H), 3.73 (t, J=11.2 Hz, 3H), 3.06 (tdd, J=13.0, 8.7, 6.9 Hz, 2H), 2.44-2.15 (m, 6H), 2.03-1.38 (m, 1H). MS (ESI⁺) m/z 604 (M+H)⁺.

### Example 922

### tert-butyl {trans-4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclohexyl}carbamate

### Example 922A

### tert-butyl (trans-4-((4-fluorobenzyl)amino)cyclohexyl)carbamate

The title compound was prepared as described in EXAMPLE 283A, substituting tert-butyl (trans-4-aminocyclohexyl)carbamate for (S)-1-cyclopropylethanamine and 4-fluorobenzaldehyde for benzaldehyde.

### Example 922B

### tert-butyl {trans-4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclohexyl}carbamate

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and tert-butyl (trans-4-((4-fluorobenzyl)amino)cyclohexyl)carbamate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆) δ 8.67 (d, J=3.1 Hz, 1H), 7.54 (s, 1H), 7.42-7.03 (m, 7H), 6.70 (dd, J=32.5, 7.9 Hz, 1H), 6.06 (t, J=4.7 Hz, 1H), 4.77-4.12 (m, 4H), 3.24-2.91 (m, 3H), 2.62 (t, J=4.2 Hz, 3H), 2.56-2.39 (m, 2H), 1.92-1.42 (m, 7H), 1.42-1.13 (m, 9H). MS (ESI): 660 (M+Na)⁺.

### Example 923

### N-(trans-4-aminocyclohexyl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 302, substituting tert-butyl {trans-4-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]cyclohexyl}carbamate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 8.96 (d, J=13.0 Hz, 1H), 8.27-7.86 (m, 2H), 7.70-6.90 (m, 4H), 6.24 (s, 1H), 4.84-4.00 (m, 5H), 3.18-2.79 (m, 3H), 2.60 (d, J=3.2 Hz, 3H), 2.03-1.82 (m, 2H), 1.74-1.25 (m, 6H). MS (ESI): 538 (M+H)⁺.

### Example 924

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-{trans-4-[(methylsulfonyl)amino]cyclohexyl}acetamide

The title compound was prepared as described in EXAMPLE 308, substituting N-(trans-4-aminocyclohexyl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide hydrochloride. ¹H NMR (400 MHz, DMSO-d₆) δ 8.66 (d, J=4.1 Hz, 1H), 7.52 (t, J=2.4 Hz, 1H), 7.39-6.84 (m, 7H), 6.04 (t, J=4.7 Hz, 1H), 4.73-4.09 (m, 4H), 3.18-2.90 (m, 2H), 2.84 (d, J=4.0 Hz, 3H), 2.61 (t, J=4.2 Hz, 3H), 2.47 (p, J=1.8 Hz, 1H), 1.94-1.75 (m, 2H), 1.67-1.46 (m, 4H), 1.45-1.20 (m, 1H). MS (ESI): 616 (M+H)⁺.

### Example 925

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-(trans-4-{[(trifluoromethyl)sulfonyl]amino}cyclohexyl)acetamide

The title compound was prepared as described in EXAMPLE 308, substituting N-(trans-4-aminocyclohexyl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide hydrochloride and trifluoromethanesulfonyl chloride for methanesulfonyl chloride. ¹H NMR (501 MHz, DMSO-d₆) δ 9.38 (s, 1H), 8.67 (d, J=5.5 Hz, 1H), 7.54 (dd, J=7.2, 1.8 Hz, 1H), 7.40-7.01 (m, 6H), 6.16-5.96 (m, 1H), 4.80-4.17 (m, 4H), 3.20-2.91 (m, 2H), 2.62 (t, J=4.8 Hz, 3H), 2.55-2.36 (m, 2H), 1.91-1.74 (m, 2H), 1.74-1.33 (m, 6H). MS (ESI): 670 (M+H)⁺.

### Example 926

### N-(4-fluorobenzyl)-2-[(4R)-7′-hydroxy-2,5-dioxo-2′,3′-dihydro-H-spiro[imidazolidine-4,1′-inden]-1-yl]-N-[(2 S)-1,1,1-trifluoropropan-2-yl]acetamide

### Example 926A

### 2-((R)-2,5-dioxo-7′-(4,4,5,5-tetramethyl-1,3,2-dioxaborolan-2-yl)-2′,3′-dihydrospiro[imidazolidine-4,1′-inden]-1-yl)-N-(4-fluorobenzyl)-N—((S)-1,1,1-trifluoropropan-2-yl)acetamide

The title compound was prepared as described in EXAMPLE 353A, substituting 2-[(4R)-7′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-((R)-5-bromo-2′,4′-dioxo-2,3-dihydrospiro[indene-1,5′-oxazolidin]-3′-yl)-N—((S)-1-cyclopropylethyl)-N-(4-fluorobenzyl)acetamide.

### Example 926B

### N-(4-fluorobenzyl)-2-[(4R)-7′-hydroxy-2,5-dioxo-2′,3′-dihydro-1H-spiro[imidazolidine-4,1′-inden]-1-yl]-N-[(2 S)-1,1,1-trifluoropropan-2-yl]acetamide

A solution of 2-((R)-2,5-dioxo-7′-(4,4,5,5-tetramethyl-1,3,2-dioxaborolan-2-yl)-2′,3′-dihydrospiro[imidazolidine-4,1′-inden]-1-yl)-N-(4-fluorobenzyl)-N—((S)-1,1,1-trifluoropropan-2-yl)acetamide (0.02 g, 0.034 mmol) in methanol (0.3 mL) was treated with hydrogen peroxide (8.85 mg, 0.078 mmol) (30% in water) and the reaction mixture was stirred at room temperature for 20 hours. The reaction mixture was directly purified by preparative reverse-phase HPLC to afford the title compound. ¹H NMR (400 MHz, DMSO-d₆, mixture of rotamers) δ ppm 9.65-9.48 (m, 1H), 8.52 (d, J=2.7 Hz, 1H), 7.36 (dd, J=8.5, 5.3 Hz, 1H), 7.29-6.98 (m, 4H), 6.69 (t, J=6.5 Hz, 1H), 6.54 (dd, J=20.6, 8.0 Hz, 1H), 5.42-5.01 (m, 2H), 4.95-4.60 (m, 2H), 4.41-4.23 (m, 1H), 4.02 (d, J=16.9 Hz, 1H), 3.08-2.80 (m, 2H), 2.45-2.36 (m, 1H), 2.18-2.03 (m, 1H), 1.29 (dd, J=27.0, 6.9 Hz, 3H). MS (ESI⁺) m/z 480 (M+H)⁺.

### Example 927

### tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetate

The title compound was prepared as described in EXAMPLE 894, substituting tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate for 2-[2,5-dioxo-4′-(1,2,3,6-tetrahydropyridin-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide. ¹H NMR (400 MHz, methanol-d₄) δ 7.91 (d, J=3.3 Hz, 1H), 7.78 (d, J=2.6 Hz, 1H), 7.47 (s, 1H), 7.45-7.30 (m, 3H), 7.23 (ddd, J=11.7, 5.9, 2.4 Hz, 1H), 7.09 (td, J=8.8, 2.5 Hz, 1H), 6.97 (ddt, J=8.8, 6.4, 2.9 Hz, 1H), 4.68-4.46 (m, 4H), 4.37-4.15 (m, 1H), 3.88 (d, J=1.9 Hz, 3H), 3.86-3.67 (m, 1H), 3.12 (p, J=7.8 Hz, 2H), 2.80-2.61 (m, 1H), 2.38-2.17 (m, 2H), 2.06 (dd, J=10.7, 7.0 Hz, 1H), 1.76 (t, J=12.6 Hz, 2H), 1.68-1.48 (m, 2H), 1.45-1.36 (m, 1H), 1.35-1.21 (m, 2H), 1.21-0.99 (m, 1H). MS (ESI⁺) m/z 644 (M+H)⁺.

### Example 928

### {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetic acid

The title compound was prepared as described in EXAMPLE 302, substituting tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, methanol-d₄) δ 7.92 (d, J=3.3 Hz, 1H), 7.78 (d, J=2.6 Hz, 1H), 7.48 (s, 1H), 7.45-7.30 (m, 3H), 7.28-7.18 (m, 1H), 7.09 (td, J=8.7, 2.7 Hz, 1H), 6.97 (td, J=8.8, 3.0 Hz, 1H), 4.70-4.46 (m, 3H), 4.41-4.14 (m, 1H), 3.89 (d, J=1.9 Hz, 3H), 3.85-3.72 (m, 1H), 3.12 (qd, J=7.8, 3.7 Hz, 2H), 2.71 (dtt, J=9.9, 7.4, 4.8 Hz, 2H), 2.42-2.23 (m, 2H), 2.13 (dd, J=10.7, 7.0 Hz, 2H), 1.78 (q, J=12.7, 10.9 Hz, 2H), 1.72-1.39 (m, 4H), 1.20-1.00 (m, 1H). MS (ESI⁺) m/z 588 (M+H)⁺.

### Example 929

### tert-butyl 6-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]-2-azaspiro[3.3]heptane-2-carboxylate

### Example 929A

### tert-butyl 6-((4-fluorobenzyl)amino)-2-azaspiro[3.3]heptane-2-carboxylate

The title compound was prepared as described in EXAMPLE 283A, substituting tert-butyl 6-((4-fluorobenzyl)amino)-2-azaspiro[3.3]heptane-2-carboxylate for (S)-1-cyclopropylethanamine and 4-fluorobenzaldehyde for benzaldehyde.

### Example 929B

### tert-butyl 6-[(4-fluorobenzyl)({(1R)-5-[(methylcarbamoyl)amino]-2′,4′-dioxo-2,3-dihydro-3′H-spiro[indene-,5′-[1,3]oxazolidin]-3′-yl}acetyl)amino]-2-azaspiro[3.3]heptane-2-carboxylate

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and tert-butyl 6-((4-fluorobenzyl)amino)-2-azaspiro[3.3]heptane-2-carboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆) δ 8.65 (d, J=3.2 Hz, 1H), 7.57-7.44 (m, 1H), 7.35-7.02 (m, 5H), 6.15-5.94 (m, 1H), 4.80-4.34 (m, 4H), 4.26 (s, 1H), 3.16-2.90 (m, 2H), 2.67-2.54 (m, 4H), 2.47 (p, J=1.9 Hz, 2H), 2.43-2.14 (m, 3H), 1.32 (s, 9H). MS (ESI): 658 (M+Na)⁺.

### Example 930

### N-(2-azaspiro[3.3]hept-6-yl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 302, substituting tert-butyl 6-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]-2-azaspiro[3.3]heptane-2-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (501 MHz, DMSO-d₆) δ 8.76 (d, J=12.6 Hz, 1H), 8.56 (s, 2H), 7.50 (dd, J=22.9, 1.7 Hz, 1H), 7.37-7.01 (m, 6H), 6.17 (s, 1H), 4.93-4.39 (m, 4H), 4.07-3.90 (m, 2H), 3.82 (t, J=6.2 Hz, 2H), 3.22-2.89 (m, 2H), 2.62 (d, J=3.9 Hz, 3H), 2.53-2.41 (m, 3H), 2.41-2.22 (m, 1H). MS (ESI): 536 (M+H)⁺.

### Example 931

### 2-[(1 S,3R)-5-bromo-3-hydroxy-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

### Example 931A

### (1 S,3R)-5-bromo-3-hydroxy-2,3-dihydrospiro[indene-1,5′-oxazolidine]-2′,4′-dione

The title compound was prepared as described in EXAMPLE 698, substituting (S)-5-bromospiro[indene-1,5′-oxazolidine]-2′,3,4′(2H)-trione for N-(4-fluorobenzyl)-N—((S)-1,1,1,1-trifluoropropan-2-yl)-2-((S)-2,3′,5-trioxo-2′,3′-dihydrospiro[imidazolidine-4,1′-inden]-1-yl)acetamide.

### Example 931B

### 2-[(1 S,3R)-5-bromo-3-hydroxy-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 281F, substituting (1S,3R)-5-bromo-3-hydroxy-2,3-dihydrospiro[indene-1,5′-oxazolidine]-2′,4′-dione for (R)-5-bromo-2,3-dihydrospiro[indene-1,5′-oxazolidine]-2′,4′-dione. ¹H NMR (400 MHz, DMSO-d₆) δ 7.94 (s, 1H), 7.66 (d, J=1.9 Hz, 1H), 7.57 (dd, J=8.3, 1.9 Hz, 1H), 7.32 (dd, J=14.8, 8.3 Hz, 2H), 7.11 (t, J=8.8 Hz, 2H), 5.53 (d, J=5.7 Hz, 1H), 5.18 (q, J=6.1 Hz, 1H), 4.70 (dd, J=78.2, 18.5 Hz, 4H), 3.02 (dd, J=14.2, 7.0 Hz, 1H), 2.31 (dd, J=14.2, 5.4 Hz, 1H), 1.36 (d, J=7.0 Hz, 3H). MS (ESI⁺) m/z 560 (M+H)⁺.

### Example 932

### N-[(1-acetylazetidin-3-yl)methyl]-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 346, substituting N-(azetidin-3-ylmethyl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide for 2-(5′-amino-2,5-dioxo-1′,3′-dihydrospiro[imidazolidine-4,2′-inden]-1-yl)-N—((S)-1-cyclopropylethyl)-N-(4-fluorobenzyl)acetamide. ¹H NMR (400 MHz, DMSO-d₆, 120° C.) δ ppm 8.33-8.21 (m, 1H), 7.54-7.45 (m, 1H), 7.44-7.06 (m, 6H), 5.92-5.78 (m, 1H), 4.62 (brs, 2H), 4.55-4.39 (m, 2H), 3.90 (s, 2H), 3.71-3.50 (m, 4H), 3.18-3.05 (m, 1H), 3.05-2.94 (m, 1H), 2.81-2.76 (m, 1H), 2.72-2.59 (m, 4H), 2.47-2.40 (m, 1H), 1.67 (s, 3H). MS (ESI⁺) m/z 552 (M+H)⁺.

### Example 933

### N-(4-fluorobenzyl)-N-[(2S)-1,1,1-trifluoropropan-2-yl]-2-(2′,3,4′-trioxo-1,2,3,4,8,9-hexahydro-3′H-spiro[cyclopenta[f]quinazoline-7,5′-[1,3]oxazolidin]-3′-yl)acetamide

### Example 933A

### 2-(5-amino-4-(aminomethyl)-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide

2-(5-Amino-4-cyano-2′,4′-dioxo-2,3-dihydro-3′H-spiro[indene-1,5′-[1,3]oxazolidin]-3′-yl)-N-(4-fluorobenzyl)-N-[(2S)-1,1,1-trifluoropropan-2-yl]acetamide (175 mg, 0.347 mmol) and acetic acid (10 ml) were added to Raney nickel 2800, water slurry (350 mg, 2.68 mmol) in a 50 ml pressure bottle and the mixture was shaken for 1 hour at 50 psi hydrogen at room temperature. Filtration and concentration provided the title compound.

### Example 933B

### N-(4-fluorobenzyl)-N-[(2S)-1,1,1-trifluoropropan-2-yl]-2-(2′,3,4′-trioxo-1,2,3,4,8,9-hexahydro-3′H-spiro[cyclopenta[f]quinazoline-7,5′-[1,3]oxazolidin]-3′-yl)acetamide

The title compound was prepared as described in EXAMPLE 282, substituting 2-(5-amino-4-(aminomethyl)-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and excluding 3,3-difluoroazetidine hydrochloride. ¹H NMR (400 MHz, DMSO-d₆) δ 9.11 (t, J=2.6 Hz, 1H), 7.35 (dd, J=8.5, 5.3 Hz, 2H), 7.26-7.02 (m, 3H), 6.87 (d, J=2.0 Hz, 1H), 6.66 (m, 1H), 5.43-5.30 (m, 0.6H), 5.08-4.66 (m, 2.4H), 4.59-4.40 (m, 1H), 4.29 (s, 2H), 4.23-4.12 (m, 1H), 2.94 (td, J=18.0, 17.2, 9.2 Hz, 2H), 2.67-2.40 (m, 2H), 1.33 (ddd, J=20.9, 7.2, 3.6 Hz, 3H). MS (ESI⁺) m/z 593 (M+CH₃CN+NH₄)⁺.

### Example 934

### ethyl [(3-{[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]methyl}azetidin-1-yl)sulfonyl]carbamate

The title compound was prepared as described in EXAMPLE 678, substituting N-(azetidin-3-ylmethyl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide for N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide. ¹H NMR (400 MHz, DMSO-d₆, 120° C.) δ ppm 8.34-8.21 (m, 1H), 7.53-7.43 (m, 1H), 7.35-7.20 (m, 3H), 7.19-7.06 (m, 2H), 6.37 (brs, 2H), 5.91-5.77 (m, 1H), 4.61 (brs, 2H), 4.48 (brs, 2H), 3.84-3.74 (m, 1H), 3.72-3.54 (m, 4H), 3.50-3.40 (m, 3H), 3.19-2.94 (m, 2H), 2.79-2.63 (m, 5H), 2.51-2.40 (m, 1H), 1.06 (t, J=7.0 Hz, 3H). MS (ESI⁺) m/z 661 (M+H)⁺.

### Example 935

### ethyl [(2-{[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]methyl}pyrrolidin-1-yl)sulfonyl]carbamate

The title compound was prepared as described in EXAMPLE 678, substituting N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-(pyrrolidin-2-ylmethyl)acetamide for N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide. ¹H NMR (400 MHz, methanol-d₄, mixture of rotamers) δ ppm 7.56-7.47 (m, 1H), 7.42-7.26 (m, 3H), 7.27-7.17 (m, 1H), 7.13 (t, J=8.7 Hz, 1H), 7.08-6.99 (m, 1H), 4.76-4.35 (m, 4H), 4.24-4.10 (m, 2H), 3.77-3.32 (m, 5H), 3.29-2.99 (m, 3H), 2.76 (s, 3H), 2.60-2.46 (m, 1H), 2.10-1.74 (m, 4H), 1.35-1.20 (m, 3H). MS (ESI⁺) m/z 675 (M+H)⁺.

### Example 936

### 2-[(4S)-2,5-dioxo-5′-(1H-pyrazol-5-yl)-2′,3′-dihydro-H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 295, substituting 2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and (1H-pyrazol-5-yl)boronic acid for 5-(4,4,5,5-tetramethyl-1,3,2-dioxaborolan-2-yl)pyridin-2-ol. ¹H NMR (400 MHz, DMSO-d₆) δ 12.56 (s, 1H), 8.37 (d, J=3.5 Hz, 1H), 7.73-7.53 (m, 3H), 7.53-7.02 (m, 5H), 6.59 (d, J=2.1 Hz, 1H), 5.34-4.91 (m, 1H), 4.79 (d, J=17.6 Hz, 1H), 4.65-4.31 (m, 2H), 4.23 (d, J=16.6 Hz, 1H), 3.07 (t, J=7.2 Hz, 2H), 2.59 (dt, J=13.3, 6.6 Hz, 1H), 2.26 (dt, J=13.3, 7.7 Hz, 1H), 1.93-1.23 (m, 6H), 0.84 (q, J=6.7 Hz, 4H). MS (ESI⁺) m/z 530 (M+H)⁺.

### Example 937

### ethyl ({6-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]-2-azaspiro[3.3]hept-2-yl}sulfonyl)carbamate

The title compound was prepared as described in EXAMPLE 678, substituting N-(2-azaspiro[3.3]hept-6-yl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide for N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide. ¹H NMR (400 MHz, DMSO-d₆) δ 11.25 (d, J=7.2 Hz, 1H), 8.67 (d, J=3.3 Hz, 1H), 7.64-7.43 (m, 1H), 7.38-7.01 (m, 6H), 6.06 (s, 1H), 4.78-4.39 (m, 4H), 4.28 (s, 1H), 4.08 (q, J=7.1 Hz, 2H), 3.19-2.91 (m, 2H), 2.62 (d, J=3.2 Hz, 4H), 2.48 (dq, J=5.6, 3.7, 2.8 Hz, 2H), 2.41-2.15 (m, 4H), 1.16 (t, J=7.1 Hz, 3H). MS (ESI): 687 (M+H)⁺.

### Example 938

### 4-[(4-fluorobenzyl){[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexanecarboxylic acid

### Example 938A

### tert-butyl 4-((4-fluorobenzyl)amino)cyclohexanecarboxylate

The title compound was prepared as described in EXAMPLE 283A, substituting tert-butyl 4-aminocyclohexanecarboxylate for (S)-1-cyclopropylethanamine and 4-fluorobenzaldehyde for benzaldehyde.

### Example 938B

### tert-butyl 4-(2-((1′S,3′R)-3′-fluoro-5′-(1-methyl-1H-pyrazol-4-yl)-2,5-dioxo-2′,3′-dihydrospiro[imidazolidine-4,1′-inden]-1-yl)-N-(4-fluorobenzyl)acetamido)cyclohexanecarboxylate

The title compound was prepared as described in EXAMPLE 306, substituting 2-((1′S,3′R)-3′-fluoro-5′-(1-methyl-1H-pyrazol-4-yl)-2,5-dioxo-2′,3′-dihydrospiro[imidazolidine-4,1′-inden]-1-yl)acetic acid for cyclopropanecarboxylic acid and tert-butyl 4-((4-fluorobenzyl)amino)cyclohexanecarboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 938C

### 4-[(4-fluorobenzyl) {[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexanecarboxylic acid

The title compound was prepared as described in EXAMPLE 302, substituting tert-butyl 4-(2-((1′S,3′R)-3′-fluoro-5′-(1-methyl-1H-pyrazol-4-yl)-2,5-dioxo-2′,3′-dihydrospiro[imidazolidine-4,1′-inden]-1-yl)-N-(4-fluorobenzyl)acetamido)cyclohexanecarboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 9.12-8.93 (m, 1H), 8.21 (d, J=5.7 Hz, 1H), 7.92 (d, J=5.2 Hz, 1H), 7.80-7.63 (m, 2H), 7.63-7.04 (m, 5H), 6.21 (q, J=5.4 Hz, 1H), 6.07 (q, J=5.2 Hz, 1H), 4.67-4.37 (m, 4H), 4.17 (s, 2H), 3.85 (d, J=3.3 Hz, 3H), 3.05 (tt, J=13.1, 6.6 Hz, 1H), 2.67 (s, 3H), 2.34 (dtd, J=27.5, 14.0, 4.4 Hz, 1H), 2.17-1.83 (m, 3H), 1.68-1.03 (m, 7H). MS (ESI⁺) m/z 592 (M+H)⁺.

### Example 939

### N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-(3-{[(trifluoromethyl)sulfonyl]amino}cyclobutyl)acetamide

The title compound was prepared as described in EXAMPLE 308, substituting N-(2-azaspiro[3.3]hept-6-yl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide hydrochloride and trifluoromethanesulfonyl chloride for methanesulfonyl chloride. ¹H NMR (500 MHz, DMSO-d₆) δ 9.87 (s, 1H), 8.68 (d, J=3.5 Hz, 1H), 7.54 (d, J=6.7 Hz, 1H), 7.37-7.03 (m, 6H), 6.06 (t, J=4.7 Hz, 1H), 5.15-4.50 (m, 3H), 4.32 (d, J=21.4 Hz, 1H), 4.01-3.86 (m, 1H), 3.19-2.91 (m, 2H), 2.63 (t, J=4.3 Hz, 5H), 2.49 (p, J=1.9 Hz, 2H), 2.38-2.02 (m, 2H). MS (ESI): 642 (M+H)⁺.

### Example 940

### tert-butyl 3-[{[(1R)-5-bromo-2′,4′-dioxo-2,3-dihydro-3′H-spiro[indene-1,5′-[1,3]oxazolidin]-3′-yl]acetyl}(4-fluorobenzyl)amino]benzoate

The title compound was prepared as described in EXAMPLE 306, substituting (R)-2-(5-bromo-2′,4′-dioxo-2,3-dihydrospiro[indene-1,5′-oxazolidin]-3′-yl)acetic acid for cyclopropanecarboxylic acid and tert-butyl 3-((4-fluorobenzyl)amino)benzoate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆, 120° C.) δ ppm 7.86 (dt, J=7.7, 1.5 Hz, 1H), 7.68-7.63 (m, 1H), 7.62-7.58 (m, 1H), 7.56-7.44 (m, 3H), 7.36 (d, J=8.2 Hz, 1H), 7.27-7.18 (m, 2H), 7.09-7.00 (m, 2H), 4.91 (s, 2H), 4.20-4.07 (m, 2H), 3.25-3.02 (m, 2H), 2.74-2.62 (m, 1H), 2.55-2.48 (m, 1H), 1.52 (s, 9H). MS (ESI⁺) m/z 623 (M−H)⁺.

### Example 941

### tert-butyl 3-[(4-fluorobenzyl) {[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]benzoate

The title compound was prepared as described in EXAMPLE 295, substituting tert-butyl 3-[{[(1R)-5-bromo-2′,4′-dioxo-2,3-dihydro-3′H-spiro[indene-1,5′-[1,3]oxazolidin]-3′-yl]acetyl}(4-fluorobenzyl)amino]benzoate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆, 120° C.) δ ppm 8.00 (s, 1H), 7.87 (dt, J=7.6, 1.5 Hz, 1H), 7.80-7.74 (m, 1H), 7.69-7.64 (m, 1H), 7.58-7.43 (m, 4H), 7.38 (d, J=8.1 Hz, 1H), 7.29-7.19 (m, 2H), 7.10-7.00 (m, 2H), 4.92 (s, 2H), 4.22-4.08 (m, 2H), 3.85 (s, 3H), 3.24-3.01 (m, 2H), 2.74-2.63 (m, 1H), 2.55-2.49 (m, 1H), 1.52 (s, 9H). MS (ESI⁺) m/z 625 (M+H)⁺.

### Example 942

### 3-[(4-fluorobenzyl) {[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]benzoic acid

The title compound was prepared as described in EXAMPLE 302, substituting tert-butyl 3-[(4-fluorobenzyl) {[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]benzoate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, methanol-d₄) δ ppm 8.07 (d, J=7.7 Hz, 1H), 8.00 (s, 1H), 7.91-7.81 (m, 2H), 7.63-7.40 (m, 5H), 7.30-7.17 (m, 2H), 7.01 (t, J=8.8 Hz, 2H), 5.07-4.91 (m, 2H), 4.23-4.07 (m, 2H), 3.92 (s, 3H), 3.28-3.17 (m, 1H), 3.17-3.07 (m, 1H), 2.83-2.71 (m, 1H), 2.61-2.50 (m, 1H). MS (ESI⁺) m/z 569 (M+H)⁺.

### Example 943

### 2-[(4S)-5′-{1-[2-(dimethylamino)ethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide

The title compound was prepared as described in EXAMPLE 295, substituting 2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N,N-dimethyl-2-(4-(4,4,5,5-tetramethyl-1,3,2-dioxaborolan-2-yl)-1H-pyrazol-1-yl)ethanamine for 5-(4,4,5,5-tetramethyl-1,3,2-dioxaborolan-2-yl)pyridin-2-ol. ¹H NMR (400 MHz, DMSO-d₆, 120° C.) δ 8.34 (s, 1H), 8.01 (s, 1H), 7.73 (s, 1H), 7.44 (d, J=1.4 Hz, 1H), 7.38 (dd, J=7.9, 1.6 Hz, 1H), 7.32 (dd, J=8.5, 5.5 Hz, 2H), 7.19 (d, J=7.9 Hz, 1H), 7.10 (t, J=8.8 Hz, 2H), 5.16 (m, 1H), 4.79 (d, J=17.5 Hz, 1H), 4.58 (d, J=17.6 Hz, 1H), 4.43 (d, J=16.6 Hz, 1H), 4.27-4.14 (m, 3H), 3.04 (t, J=7.2 Hz, 2H), 2.71 (t, J=6.6 Hz, 2H), 2.57 (dt, J=13.3, 6.7 Hz, 1H), 2.25 (dd, J=13.4, 7.6 Hz, 1H), 2.19 (s, 6H), 1.33 (d, J=7.0 Hz, 3H). MS (ESI⁺) m/z 601 (M+H)⁺.

### Example 944

### tert-butyl 4-({4-[(4S)-1-(2-{(4-fluorobenzyl)[(2S)-1,1,1-trifluoropropan-2-yl]amino}-2-oxoethyl)-2,5-dioxo-2′,3′-dihydrospiro[imidazolidine-4,1′-inden]-5′-yl]-1H-pyrazol-1-yl}methyl)piperidine-1-carboxylate

### Example 944A

### tert-butyl 4-((4-(4,4,5,5-tetramethyl-1,3,2-dioxaborolan-2-yl)-1H-pyrazol-1-yl)methyl)piperidine-1-carboxylate

To a mixture of 4-(4,4,5,5-tetramethyl-1,3,2-dioxaborolan-2-yl)-1H-pyrazole (150 mg, 0.773 mmol), tert-butyl 4-(hydroxymethyl)piperidine-1-carboxylate (166 mg, 0.773 mmol) in 3 ml toluene was added cyanomethylenetributylphosphorane (928 μl, 0.928 mmol) (1.0 M toluene solution). The mixture was stirred at 85° C. for 16 hours. The mixture was concentrated and the residue was diluted with a small amount of heptanes and purified by silica gel column chromatography to afford the title compound.

### Example 944B

### tert-butyl 4-({4-[(4S)-1-(2-{(4-fluorobenzyl)[(2S)-1,1,1-trifluoropropan-2-yl]amino}-2-oxoethyl)-2,5-dioxo-2′,3′-dihydrospiro[imidazolidine-4,1′-inden]-5′-yl]-1H-pyrazol-1-yl}methyl)piperidine-1-carboxylate

The title compound was prepared as described in EXAMPLE 295, substituting 2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and tert-butyl 4-((4-(4,4,5,5-tetramethyl-1,3,2-dioxaborolan-2-yl)-1H-pyrazol-1-yl)methyl)piperidine-1-carboxylate for 5-(4,4,5,5-tetramethyl-1,3,2-dioxaborolan-2-yl)pyridin-2-ol. ¹H NMR (400 MHz, DMSO-d₆, 120° C.) δ 7.98 (s, 1H), 7.75 (s, 1H), 7.44 (s, 1H), 7.38 (dd, J=7.9, 1.6 Hz, 1H), 7.32 (dd, J=8.5, 5.4 Hz, 2H), 7.19 (d, J=7.9 Hz, 1H), 7.09 (t, J=8.8 Hz, 2H), 5.23-5.09 (m, 1H), 4.79 (d, J=17.5 Hz, 1H), 4.58 (d, J=17.7 Hz, 1H), 4.43 (d, J=16.6 Hz, 1H), 4.22 (d, J=16.6 Hz, 1H), 4.01 (d, J=6.9 Hz, 2H), 3.89 (dt, J=13.3, 3.7 Hz, 2H), 3.04 (t, J=7.2 Hz, 2H), 2.72 (td, J=12.7, 11.9, 3.1 Hz, 2H), 2.57 (dt, J=13.3, 6.6 Hz, 1H), 2.24 (dt, J=13.3, 7.7 Hz, 1H), 2.05 (ddd, J=11.3, 7.2, 4.1 Hz, 1H), 1.54 (dd, J=12.9, 3.9 Hz, 2H), 1.38 (s, 9H), 1.33 (d, J=7.1 Hz, 3H). MS (ESI⁻) m/z 725 (M−H)⁻.

### Example 945

### 2-{(4S)-2,5-dioxo-5′-[1-(piperidin-4-ylmethyl)-1H-pyrazol-4-yl]-2′,3′-dihydro-1H-spiro[imidazolidine-4,1′-inden]-1-yl}-N-(4-fluorobenzyl)-N-[(2S)-1,1,1-trifluoropropan-2-yl]acetamide

The title compound was prepared as described in EXAMPLE 302, substituting tert-butyl 4-({4-[(4S)-1-(2-{(4-fluorobenzyl)[(2S)-1,1,1-trifluoropropan-2-yl]amino}-2-oxoethyl)-2,5-dioxo-2′,3′-dihydrospiro[imidazolidine-4,1′-inden]-5′-yl]-1H-pyrazol-1-yl}methyl)piperidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆, 120° C.) δ 8.36-8.32 (m, 1H), 8.32-8.18 (m, 1H), 8.01 (s, 1H), 7.78 (s, 1H), 7.44 (s, 1H), 7.38 (dd, J=8.0, 1.6 Hz, 1H), 7.32 (dd, J=8.5, 5.4 Hz, 2H), 7.13-7.06 (m, 2H), 5.21-5.08 (m, 1H), 4.79 (d, J=17.6 Hz, 1H), 4.58 (d, J=17.6 Hz, 1H), 4.43 (d, J=16.6 Hz, 1H), 4.22 (d, J=16.6 Hz, 1H), 4.07 (d, J=6.7 Hz, 2H), 3.27 (dt, J=12.9, 3.8 Hz, 2H), 3.04 (t, J=7.2 Hz, 2H), 2.85 (d, J=3.2 Hz, 2H), 2.57 (dt, J=13.3, 6.6 Hz, 1H), 2.30-2.12 (m, 2H), 1.76 (dd, J=14.5, 3.8 Hz, 2H), 1.44 (dtd, J=15.0, 11.8, 4.2 Hz, 2H), 1.33 (d, J=7.1 Hz, 3H). MS (ESI⁺) m/z 627 (M+H)⁺.

### Example 946

### N-(4-fluorobenzyl)-2-{6-[(1-methyl-1H-pyrazol-4-yl)amino]-1,1-dioxido-2′,5′-dioxo-1′H-spiro[1-benzothiophene-3,4′-imidazolidin]-1′-yl}-N-[(2S)-1,1,1-trifluoropropan-2-yl]acetamide

The title compound was prepared as described in EXAMPLE 284D, substituting 2-(6-bromo-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide. ¹H NMR (400 MHz, DMSO-d₆) δ ppm 9.22-9.10 (m, 1H), 8.25 (brs, 1H), 7.80-7.70 (m, 1H), 7.42-7.29 (m, 3H), 7.26-7.01 (m, 3H), 6.80 (d, J=2.2 Hz, 1H), 5.42-5.24 (m, 1H), 5.10-4.62 (m, 3H), 4.50-4.32 (m, 1H), 4.07 (dd, J=20.7, 17.0 Hz, 1H), 3.96-3.64 (m, 5H), 1.36-1.18 (m, 3H). MS (ESI⁺) m/z 609 (M+H)⁺.

### Example 947

### N-(4-fluorobenzyl)-2-[(1S)-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide

### Example 947A

### 2-((S)-5-bromo-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide

The title compound was prepared as described in EXAMPLE 281F, substituting (S)-5-bromo-2,3-dihydrospiro[indene-1,5′-oxazolidine]-2′,4′-dione for (R)-5-bromo-2,3-dihydrospiro[indene-1,5′-oxazolidine]-2′,4′-dione.

### Example 947B

### N-(4-fluorobenzyl)-2-[(1S)-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-((S)-5-bromo-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.07 (s, 1H), 7.82 (s, 1H), 7.65-7.44 (m, 2H), 7.41-7.18 (m, 2H), 7.12 (t, J=8.8 Hz, 2H), 5.17 (t, J=8.1 Hz, 1H), 4.97-4.15 (m, 4H), 3.38-2.98 (m, 2H), 2.81-2.49 (m, 4H), 1.37 (d, J=7.0 Hz, 3H). MS (ESI⁺) m/z 602 (M+H)⁺.

### Example 948

### N-(azetidin-3-yl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

### Example 948A

### benzyl 3-((4-fluorobenzyl)amino)azetidine-1-carboxylate

The title compound was prepared as described in EXAMPLE 283A, substituting benzyl 3-aminoazetidine-1-carboxylate for (S)-1-cyclopropylethanamine and 4-fluorobenzaldehyde for benzaldehyde.

### Example 948B

### (R)-benzyl 3-(N-(4-fluorobenzyl)-2-(5-(3-methylureido)-2′,4′-dioxo-2,3-dihydrospiro[indene-1,5′-oxazolidin]-3′-yl)acetamido)azetidine-1-carboxylate

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and benzyl 3-((4-fluorobenzyl)amino)azetidine-1-carboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 948C

### N-(azetidin-3-yl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide

The title compound was prepared as described in EXAMPLE 894, substituting (R)-benzyl 3-(N-(4-fluorobenzyl)-2-(5-(3-methylureido)-2′,4′-dioxo-2,3-dihydrospiro[indene-1,5′-oxazolidin]-3′-yl)acetamido)azetidine-1-carboxylate for 2-[2,5-dioxo-4′-(1,2,3,6-tetrahydropyridin-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide. ¹H NMR (400 MHz, DMSO-d₆) δ 8.71 (d, J=3.9 Hz, 1H), 7.60-7.46 (m, 1H), 7.41-7.00 (m, 6H), 6.08 (d, J=5.1 Hz, 1H), 5.09-4.22 (m, 4H), 3.56 (dd, J=23.7, 9.0 Hz, 1H), 3.15-2.90 (m, 2H), 2.62 (t, J=3.6 Hz, 5H), 2.48 (p, J=1.9 Hz, 2H). MS (ESI): 496 (M+H)⁺.

### Example 949

### ethyl ({3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]azetidin-1-yl}sulfonyl)carbamate

The title compound was prepared as described in EXAMPLE 678, substituting N-(azetidin-3-yl)-N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}acetamide for N-(4-fluorobenzyl)-2-{(1R)-5-[(methylcarbamoyl)amino]-2′,4′-dioxo-2,3-dihydro-3′H-spiro[indene-1,5′-[1,3]oxazolidin]-3′-yl}-N-[(3R)-pyrrolidin-3-yl]acetamide. ¹H NMR (400 MHz, DMSO-d₆) δ 11.42 (d, J=23.0 Hz, 1H), 8.68 (d, J=6.1 Hz, 1H), 7.53 (s, 1H), 7.39-7.05 (m, 6H), 6.06 (d, J=4.5 Hz, 1H), 5.16-4.59 (m, 4H), 4.37 (d, J=2.4 Hz, 1H), 4.30-3.91 (m, 5H), 3.19-2.89 (m, 1H), 2.62 (d, J=3.8 Hz, 4H), 2.48 (p, J=1.9 Hz, 2H), 1.29-1.09 (m, 3H). MS (ESI): 647 (M+H)⁺.

### Example 950

### 2-[(1S)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 947A. ¹H NMR (400 MHz, DMSO-d₆) δ 7.59 (d, J=1.6 Hz, 1H), 7.48 (dd, J=8.3, 1.8 Hz, 1H), 7.39-7.24 (m, 3H), 7.11 (t, J=8.7 Hz, 2H), 5.16 (s, 1H), 4.90-4.28 (m, 4H), 3.31-3.01 (m, 2H), 2.76-2.49 (m, 2H), 1.37 (d, J=7.1 Hz, 3H). MS (ESI⁺⁾ m/z 565 (M+Na)⁺.

### Example 951

### N-(4-fluorobenzyl)-2-(6-methoxy-1,1-dioxido-2′,5′-dioxo-1′H-spiro[1-benzothiophene-3,4′-imidazolidin]-1′-yl)-N-[(2S)-1,1,1-trifluoropropan-2-yl]acetamide

A 4 mL vial was charged with diacetoxypalladium (0.973 mg, 4.33 μmol), 5-(di(1-adamantyl)phosphino)-1′,3′,5′-triphenyl-1′H-[1,4′]bipyrazole (5.74 mg, 8.66 μmol) and cesium carbonate (0.041 g, 0.127 mmol). Toluene (0.173 ml), methanol (0.116 ml) and 2-(6-bromo-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide (0.04 g, 0.058 mmol) were added and the vial was sealed under nitrogen. The reaction mixture was heated at 82° C. for 2.5 hours. The reaction mixture was diluted with ethyl acetate, washed with water and brine, dried over anhydrous sodium sulfate, filtered, and concentrated. The residue was purified by reverse-phase HPLC to give the title compound. ¹H NMR (400 MHz, DMSO-d₆,120° C.) δ ppm 8.87 (brs, 1H), 7.55 (dd, J=8.7, 6.8 Hz, 1H), 7.39-7.22 (m, 4H), 7.18-7.05 (m, 2H), 5.16 (brs, 1H), 4.80 (dd, J=17.6, 5.8 Hz, 1H), 4.71-4.33 (m, 2H), 4.27 (dd, J=16.6, 4.7 Hz, 1H), 4.00-3.84 (m, 4H), 3.77 (dd, J=14.4, 4.0 Hz, 1H), 1.34 (d, J=7.0 Hz, 3H). MS (APCI⁺) m/z 561 (M+NH₄)⁺.

### Example 952

### N-(4-fluorobenzyl)-2-[(3 S)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide

The title compound was prepared as described in EXAMPLE 295, substituting 2-(6-bromo-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol; separation of the resulting diastereomeric mixture by chiral prep-SFC (Chiralcel OD-H) gave N-(4-fluorobenzyl)-2-[(3 S)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide (eluted first) and N-(4-fluorobenzyl)-2-[(3 S)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide (eluted second). ¹H NMR (400 MHz, DMSO-d₆,120° C.) δ ppm 8.21 (s, 1H), 8.00-7.86 (m, 3H), 7.61 (d, J=8.1 Hz, 1H), 7.39-7.26 (m, 2H), 7.18-7.05 (m, 2H), 5.26-5.06 (m, 1H), 4.86-4.75 (m, 1H), 4.68-4.42 (m, 2H), 4.35-4.22 (m, 1H), 3.93 (d, J=14.4 Hz, 1H), 3.86 (s, 3H), 3.77 (d, J=14.4 Hz, 1H), 1.35 (d, J=7.1 Hz, 3H). MS (ESI⁺) m/z 594 (M+H)⁺.

### Example 953

### N-(4-fluorobenzyl)-2-[(3R)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide

The title compound was prepared as described in EXAMPLE 295, substituting 2-(6-bromo-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. Separation of the resulting diastereomeric mixture by chiral prep-SFC (Chiralcel OD-H) gave N-(4-fluorobenzyl)-2-[(3S)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide (eluted first) and N-(4-fluorobenzyl)-2-[(3 S)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide (eluted second). ¹H NMR (400 MHz, DMSO-d₆,120° C.) δ ppm 8.20 (s, 1H), 7.99-7.87 (m, 3H), 7.62 (d, J=8.2 Hz, 1H), 7.38-7.26 (m, 2H), 7.17-7.06 (m, 2H), 5.25-5.10 (m, 1H), 4.81 (d, J=17.6 Hz, 1H), 4.67-4.44 (m, 2H), 4.34-4.22 (m, 1H), 3.94 (d, J=14.4 Hz, 1H), 3.86 (s, 3H), 3.75 (d, J=14.4 Hz, 1H), 1.34 (d, J=7.0 Hz, 3H). MS (ESI⁺) m/z 594 (M+H)⁺.

### Example 954

### N-(4-fluorobenzyl)-2-[(1 S,3R)-3-hydroxy-5-(1-methyl-1H-pyrazol-4-yl)-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-[(1S,3R)-5-bromo-3-hydroxy-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.02 (s, 1H), 7.77 (s, 1H), 7.62 (dd, J=1.7, 0.8 Hz, 1H), 7.55 (dd, J=8.1, 1.7 Hz, 1H), 7.42-7.24 (m, 3H), 7.11 (t, J=8.8 Hz, 2H), 5.38 (d, J=5.8 Hz, 1H), 5.18 (p, J=7.9, 6.9 Hz, 2H), 4.91-4.20 (m, 4H), 3.85 (s, 3H), 3.02 (dd, J=14.2, 7.0 Hz, 1H), 2.32 (dd, J=14.2, 5.1 Hz, 1H), 1.36 (d, J=7.0 Hz, 3H). MS (ESI⁺) m/z 561 (M+H)⁺.

### Example 955

### N-benzyl-N-(1-cyclopropylethyl)-2-(2,5-dioxo-2′,3′-dihydro-1H-spiro[imidazolidine-4, 1′-inden]-1-yl)acetamide

The title compound was prepared using the appropriate starting materials and a procedure similar to those described herein. ¹H NMR (400 MHz, DMSO-d₆,120° C.) δ 7.33-7.19 (m, 8H), 7.18 (d, J=2.7 Hz, 1H), 4.65 (s, 2H), 4.23 (s, 2H), 3.04 (t, J=7.2 Hz, 2H), 2.86-2.79 (m, 1H), 2.57 (dd, J=13.4, 6.6 Hz, 1H), 2.22 (dt, J=13.3, 7.7 Hz, 1H), 1.14 (d, J=6.7 Hz, 3H), 1.05-0.89 (m, 1H), 0.55-0.41 (m, 1H), 0.30-0.16 (m, 3H). MS (ESI⁺) m/z 350 (M+H)⁺.

### Example 956

### N-(1-cyclopropylethyl)-2-(2,5-dioxo-2′,3′-dihydro-1H-spiro[imidazolidine-4,1′-inden]-1-yl)-N-(1H-imidazol-4-ylmethyl)acetamide

The title compound was prepared using the appropriate starting materials and a procedure similar to those described herein. ¹H NMR (400 MHz, DMSO-d₆,120° C.) δ 8.33 (s, 1H), 7.34-7.16 (m, 1H), 4.58 (s, 1H), 4.42-4.23 (m, 1H), 3.05 (d, J=7.3 Hz, 4H), 2.88-2.54 (m, 7H), 2.37-0.99 (m, 2H), 0.99-0.19 (m, 1H). MS (ESI⁺) m/z 408 (M+H)⁺.

### Example 957

### N-[(1S)-3′-(2-{[(1S)-1-cyclopropylethyl](4-fluorobenzyl)amino}-2-oxoethyl)-2′,4′-dioxo-2,3-dihydrospiro[indene-1,5′-[1,3]oxazolidin]-5-yl]-D-valinamide

The title compound was prepared using the appropriate starting materials and a procedure similar to those described herein. ¹H NMR (400 MHz, DMSO-d₆,120° C.) δ 7.64 (d, J=1.6 Hz, 1H), 7.50-7.30 (m, 2H), 7.08 (t, J=8.6 Hz, 1H), 4.66 (s, 1H), 4.40 (s, 1H), 3.79-3.50 (m, 1H), 3.27-2.99 (m, 4H), 2.69 (ddd, J=14.8, 8.6, 6.4 Hz, 3H), 2.21 (dq, J=13.4, 6.7 Hz, 1H), 1.18 (d, J=6.7 Hz, 2H), 1.08-0.90 (m, 4H), 0.50 (ddd, J=10.6, 8.0, 5.5 Hz, 1H), 0.25 (ddt, J=24.7, 10.1, 5.5 Hz, 2H). MS (ESI⁺) m/z 551 (M+H)⁺.

### Example 958

### N-benzyl-N-(1-cyclopropylethyl)-2-(2,2-dimethyl-2′,5′-dioxo-1′H-spiro[1-benzofuran-3,4′-imidazolidin]-1′-yl)acetamide

The title compound was prepared using the appropriate starting materials and a procedure similar to those described herein. ¹H NMR (400 MHz, DMSO-d₆,120° C.) δ 7.38-7.16 (m, 7H), 4.65 (d, J=2.2 Hz, 2H), 4.23 (d, J=5.4 Hz, 2H), 1.59 (d, J=28.8 Hz, 1H), 1.43 (d, J=28.8 Hz, 6H), 1.14 (dd, J=6.8, 2.3 Hz, 3H), 0.24 (ddd, J=17.9, 9.0, 5.0 Hz, 3H). MS (ESI⁺) m/z 380 (M+H)⁺.

### Example 959

### N-benzyl-N-(1-cyclopropylethyl)-2-(1′,3-dimethyl-2,2′,5-trioxo-1′,2′-dihydro-1H-spiro[imidazolidine-4,3′-indol]-1-yl)acetamide

The title compound was prepared using the appropriate starting materials and a procedure similar to those described herein. ¹H NMR (400 MHz, DMSO-d₆,120° C.) δ 7.37-7.09 (m, 8H), 4.65 (s, 2H), 4.32 (d, J=4.8 Hz, 2H), 3.21 (s, 3H), 2.60 (s, 3H), 1.14 (d, J=6.7 Hz, 3H), 0.23 (ddd, J=19.0, 9.4, 5.1 Hz, 3H). MS (ESI⁺) m/z 393 (M+H)⁺.

### Example 960

### N-benzyl-N-(1-cyclopropylethyl)-2-(2,5-dioxo-2′,3′-dihydro-1H-spiro[imidazolidine-4,1′-inden]-1-yl)-2-methylpropanamide

The title compound was prepared using the appropriate starting materials and a procedure similar to those described herein. ¹H NMR (400 MHz, DMSO-d₆,120° C.) δ 8.28 (s, 1H), 7.33-7.14 (m, 8H), 7.05 (d, J=7.6 Hz, 1H), 4.61 (d, J=5.0 Hz, 2H), 3.67-3.41 (m, 3H), 3.20 (s, 1H), 3.14-2.97 (m, 3H), 2.28-2.11 (m, 1H), 1.18-0.80 (m, 7H), 0.46 (s, 1H), 0.31-0.11 (m, 3H). MS (ESI⁺) m/z 446 (M+H)⁺.

### Example 961

### N-benzyl-N-(1-cyclopropylethyl)-2-[2,5-dioxo-3-(prop-2-en-1-yl)-2′,3′-dihydro-1H-spiro[imidazolidine-4,1′-inden]-1-yl]acetamide

The title compound was prepared using the appropriate starting materials and a procedure similar to those described herein. ¹H NMR (400 MHz, DMSO-d₆,120° C.) δ 7.38-7.16 (m, 9H), 5.64 (ddt, J=16.4, 10.3, 5.8 Hz, 1H), 5.09-4.89 (m, 2H), 4.66 (s, 2H), 4.29 (t, J=4.5 Hz, 2H), 3.86 (ddt, J=16.3, 5.5, 1.8 Hz, 1H), 3.67-3.41 (m, 2H), 3.07 (t, J=7.3 Hz, 2H), 2.35 (dt, J=14.2, 7.3 Hz, 1H), 1.15 (d, J=6.7 Hz, 3H), 0.96 (ddt, J=11.5, 7.8, 4.1 Hz, 1H), 0.48 (ddd, J=11.1, 6.3, 2.3 Hz, 1H), 0.24 (ddt, J=18.8, 8.7, 2.6 Hz, 3H). MS (ESI⁺) m/z 390 (M+H)⁺.

### Example 962

### N-(1-cyclopropylethyl)-2-(2,5-dioxo-2′,3′-dihydro-1H-spiro[imidazolidine-4,1′-inden]-1-yl)-N-(2,2,2-trifluoroethyl)acetamide

The title compound was prepared using the appropriate starting materials and a procedure similar to those described herein. ¹H NMR (400 MHz, DMSO-d₆,120° C.) δ 8.32 (s, 1H), 7.34-7.16 (m, 4H), 4.44-4.11 (m, 4H), 3.49-3.33 (m, 1H), 3.04 (t, J=7.2 Hz, 2H), 2.57 (dd, J=13.3, 6.7 Hz, 1H), 2.23 (dt, J=13.3, 7.7 Hz, 1H), 1.26 (d, J=6.7 Hz, 3H), 1.07 (tt, J=8.7, 5.0 Hz, 1H), 0.68-0.22 (m, 4H). MS (ESI⁺) m/z 342 (M+H)⁺.

### Example 963

### N-(1-cyclopropylethyl)-2-(2,5-dioxo-2′,3′-dihydro-1H-spiro[imidazolidine-4,1′-inden]-1-yl)-N-[3-(trifluoromethoxy)benzyl]acetamide

The title compound was prepared using the appropriate starting materials and a procedure similar to those described herein. ¹H NMR (400 MHz, DMSO-d₆,120° C.) δ 7.48-7.10 (m, 8H), 4.79-4.55 (m, 2H), 4.36-4.16 (m, 2H), 3.04 (t, J=7.2 Hz, 2H), 2.56 (dd, J=13.4, 6.7 Hz, 1H), 1.16 (dd, J=6.7, 2.0 Hz, 3H), 0.33-0.12 (m, 3H). MS (ESI⁺) m/z 502 (M+H)⁺.

### Example 964

### N-(but-2-yn-1-yl)-N-(1-cyclopropylethyl)-2-(2,5-dioxo-2′,3′-dihydro-H-spiro[imidazolidine-4,1′-inden]-1-yl)acetamide

The title compound was prepared using the appropriate starting materials and a procedure similar to those described herein. ¹H NMR (400 MHz, DMSO-d₆,120° C.) δ 8.27 (s, 1H), 7.33-7.16 (m, 4H), 4.31 (d, J=1.5 Hz, 2H), 4.13 (dq, J=12.7, 2.5 Hz, 2H), 3.04 (t, J=7.2 Hz, 2H), 2.57 (dd, J=13.4, 6.7 Hz, 1H), 2.22 (dt, J=13.3, 7.6 Hz, 1H), 1.77 (t, J=2.4 Hz, 3H), 1.23 (dd, J=6.8, 1.6 Hz, 3H), 0.36 (ddt, J=42.6, 8.6, 4.4 Hz, 4H). MS (ESI⁺) m/z 312 (M+H)⁺.

### Example 965

### N-(1-cyclopropylethyl)-N-{[6-(difluoromethoxy)naphthalen-2-yl]methyl}-2-(2,5-dioxo-2′,3′-dihydro-1H-spiro[imidazolidine-4,1′-inden]-1-yl)acetamide

The title compound was prepared using the appropriate starting materials and a procedure similar to those described herein. ¹H NMR (400 MHz, DMSO-d₆,120° C.) δ 7.85 (dd, J=11.2, 8.7 Hz, 3H), 7.37-7.11 (m, 5H), 4.81 (s, 2H), 4.39-4.20 (m, 2H), 3.04 (t, J=7.2 Hz, 2H), 2.57 (dd, J=13.3, 6.7 Hz, 1H), 2.22 (dt, J=13.3, 7.6 Hz, 1H), 1.18 (d, J=6.7 Hz, 3H), 1.06-0.91 (m, 1H), 0.46 (dd, J=11.0, 4.8 Hz, 1H), 0.24 (ddd, J=10.0, 4.8, 2.6 Hz, 3H). MS (ESI⁺) m/z 534 (M+H)⁺.

### Example 966

### N-benzyl-N-(1-cyclopropylethyl)-2-[5′-(methylsulfonyl)-2,5-dioxo-2′,3′-dihydro-1H-spiro[imidazolidine-4,1′-inden]-1-yl]acetamide

The title compound was prepared using the appropriate starting materials and a procedure similar to those described herein. ¹H NMR (400 MHz, DMSO-d₆,120° C.) δ 8.44 (s, 1H), 7.89-7.74 (m, 2H), 7.51 (d, J=8.1 Hz, 1H), 7.35-7.15 (m, 4H), 4.65 (s, 2H), 4.25 (s, 2H), 3.59 (s, 1H), 3.14 (d, J=4.4 Hz, 5H), 2.63 (dt, J=13.4, 6.6 Hz, 1H), 2.31 (dt, J=13.5, 7.8 Hz, 1H), 1.14 (dd, J=6.7, 1.6 Hz, 3H), 0.96 (dp, J=13.7, 7.0, 6.1 Hz, 1H), 0.47 (d, J=8.9 Hz, 1H), 0.25 (t, J=11.5 Hz, 3H). MS (ESI⁺) m/z 428 (M+H)⁺.

### Example 967

### N-benzyl-N-(1-cyclobutylethyl)-2-(7′-methoxy-2,5-dioxo-2′,3′-dihydro-1H-spiro[imidazolidine-4,1′-inden]-1-yl)acetamide

The title compound was prepared using the appropriate starting materials and a procedure similar to those described herein. ¹H NMR (400 MHz, DMSO-d₆,120° C.) δ 8.04 (s, 1H), 7.34-7.14 (m, 6H), 6.81 (dd, J=32.4, 7.8 Hz, 2H), 4.60-4.09 (m, 5H), 3.67 (d, J=1.7 Hz, 3H), 3.16-2.90 (m, 2H), 2.62-2.54 (m, 1H), 2.18 (ddd, J=13.2, 8.5, 4.5 Hz, 1H), 1.95 (s, 1H), 1.71 (s, 5H), 0.99 (d, J=6.6 Hz, 3H). MS (ESI⁺) m/z 462 (M+H)⁺.

### Example 968

### N-benzyl-N-(1-cyclopropylethyl)-2-(1-methyl-2′,5′-dioxo-5,6-dihydro-1H,1′H-spiro[cyclopenta[c]pyrazole-4,4′-imidazolidin]-1′-yl)acetamide

The title compound was prepared using the appropriate starting materials and a procedure similar to those described herein. ¹H NMR (400 MHz, DMSO-d₆,120° C.) δ 8.12 (s, 1H), 7.38-7.15 (m, 6H), 4.64 (s, 2H), 4.20 (d, J=3.1 Hz, 2H), 3.69 (s, 3H), 3.51 (s, 1H), 2.97 (ddd, J=13.3, 8.4, 4.8 Hz, 1H), 2.61 (ddd, J=13.4, 8.3, 5.2 Hz, 1H), 1.14 (d, J=6.7 Hz, 3H), 0.94 (ddt, J=13.3, 8.5, 4.1 Hz, 1H), 0.47 (dq, J=7.6, 5.3, 4.8 Hz, 1H), 0.23 (dtt, J=16.6, 10.4, 5.1 Hz, 3H). MS (ESI⁺) m/z 422 (M+H)⁺.

### Example 969

### N-benzyl-N-(1-cyclopropylethyl)-2-[5′-(hydroxymethyl)-3-methyl-2,5-dioxo-2′,3′-dihydro-1H-spiro[imidazolidine-4,1′-inden]-1-yl]acetamide

The title compound was prepared using the appropriate starting materials and a procedure similar to those described herein. ¹H NMR (400 MHz, DMSO-d₆,120° C.) δ 7.38-7.05 (m, 8H), 4.65 (s, 2H), 4.50 (s, 2H), 4.38-4.18 (m, 2H), 3.07 (t, J=7.3 Hz, 2H), 2.61 (s, 3H), 1.14 (dd, J=6.9, 1.1 Hz, 3H), 0.32-0.14 (m, 3H). MS (ESI⁺) m/z 462 (M+H)⁺.

### Example 970

### N-[(1S)-1-cyclobutylethyl]-2-[(4R)-2,5-dioxo-2′,3′-dihydro-1H-spiro[imidazolidine-4,1′-inden]-1-yl]-N-(prop-2-yn-1-yl)acetamide

The title compound was prepared using the appropriate starting materials and a procedure similar to those described herein. ¹H NMR (400 MHz, DMSO-d₆,120° C.) δ 7.35-7.17 (m, 5H), 4.46-4.27 (m, 2H), 4.24-3.90 (m, 3H), 3.04 (t, J=7.2 Hz, 3H), 2.66-2.54 (m, 2H), 2.23 (dt, J=13.3, 7.6 Hz, 1H), 1.96 (dq, J=6.2, 3.4 Hz, 2H), 1.90-1.60 (m, 5H), 1.14-1.01 (m, 4H). MS (ESI⁺) m/z 380 (M+H)⁺.

### Example 971

### N³-[(1S)-1-cyclobutylethyl]-N³-{[(4R)-2,5-dioxo-2′,3′-dihydro-1H-spiro[imidazolidine-4,1′-inden]-1-yl]acetyl}-beta-alaninamide

The title compound was prepared using the appropriate starting materials and a procedure similar to those described herein. ¹H NMR (400 MHz, DMSO-d₆,120° C.) δ 8.28 (s, 1H), 7.33-7.16 (m, 4H), 6.63 (s, 2H), 4.39-4.18 (m, 2H), 4.00 (s, 1H), 3.37 (q, J=8.2 Hz, 2H), 3.04 (t, J=7.2 Hz, 3H), 2.60 (s, 3H), 2.41-2.16 (m, 4H), 2.13-1.46 (m, 7H), 1.06 (d, J=6.6 Hz, 3H). MS (ESI⁺) m/z 413 (M+H)⁺.

### Example 972

### N-(biphenyl-4-ylmethyl)-N-[(1S)-1-cyclobutylethyl]-2-[(4R)-2,5-dioxo-2′,3′-dihydro-1H-spiro[imidazolidine-4,1′-inden]-1-yl]acetamide

The title compound was prepared using the appropriate starting materials and a procedure similar to those described herein. ¹H NMR (400 MHz, DMSO-d₆,120° C.) δ 7.73-7.52 (m, 4H), 7.48-7.15 (m, 8H), 4.64-4.15 (m, 5H), 3.05 (t, J=7.2 Hz, 2H), 2.57 (dt, J=13.4, 6.7 Hz, 1H), 2.23 (dt, J=13.3, 7.7 Hz, 1H), 1.98 (dq, J=8.6, 4.2 Hz, 1H), 1.83-1.59 (m, 5H), 1.03 (d, J=6.7 Hz, 3H). MS (ESI⁺) m/z 508 (M+H)⁺.

### Example 973

### N²-[(1S)-1-cyclobutylethyl]-N²-{[(4R)-2,5-dioxo-2′,3′-dihydro-1H-spiro[imidazolidine-4,1′-inden]-1-yl]acetyl}glycinamide

The title compound was prepared using the appropriate starting materials and a procedure similar to those described herein. ¹H NMR (400 MHz, DMSO-d₆,120° C.) δ 8.26 (s, 1H), 7.33-7.16 (m, 4H), 6.71 (s, 1H), 4.28 (s, 3H), 3.91-3.64 (m, 2H), 3.04 (t, J=7.2 Hz, 3H), 2.22 (dt, J=13.3, 7.6 Hz, 1H), 2.00 (dd, J=7.8, 4.3 Hz, 1H), 1.82 (d, J=61.8 Hz, 5H), 1.30-0.99 (m, 3H). MS (ESI⁺) m/z 382 (M+H)⁺.

### Example 974

### N-[(1S)-1-cyclobutylethyl]-2-[(4R)-2,5-dioxo-2′,3′-dihydro-1H-spiro[imidazolidine-4,1′-inden]-1-yl]-N-[(1-methyl-2-oxo-1,2-dihydropyridin-4-yl)methyl]acetamide

The title compound was prepared using the appropriate starting materials and a procedure similar to those described herein. ¹H NMR (400 MHz, DMSO-d₆,120° C.) δ 8.30 (s, 1H), 7.47 (d, J=7.0 Hz, 1H), 7.33-7.15 (m, 4H), 6.20 (s, 1H), 6.04 (d, J=7.1 Hz, 1H), 4.39-4.08 (m, 5H), 3.36 (s, 3H), 3.04 (t, J=7.2 Hz, 3H), 2.57 (dd, J=13.3, 6.7 Hz, 1H), 2.23 (dt, J=13.3, 7.7 Hz, 1H), 1.99 (s, 1H), 1.89-1.62 (m, 5H), 1.03 (d, J=6.6 Hz, 3H). MS (ESI⁺) m/z 463 (M+H)⁺.

### Example 975

### N-benzyl-N-[(1R)-1-cyclopropylethyl]-2-[(4R)-2,5-dioxo-2′,3′-dihydro-H-spiro[imidazolidine-4,1′-inden]-1-yl]acetamide

The title compound was prepared using the appropriate starting materials and a procedure similar to those described herein. ¹H NMR (400 MHz, DMSO-d₆,120° C.) δ 7.38-7.11 (m, 8H), 4.65 (s, 2H), 4.23 (s, 2H), 3.04 (t, J=7.2 Hz, 2H), 1.14 (d, J=6.7 Hz, 3H), 0.32-0.14 (m, 3H). MS (ESI⁺) m/z 350 (M+H)⁺.

### Example 976

### N-benzyl-N-[(1S)-1-cyclopropylethyl]-2-[(4 S)-2,5-dioxo-2′,3′-dihydro-1H-spiro[imidazolidine-4, 1′-inden]-1-yl]acetamide

The title compound was prepared using the appropriate starting materials and a procedure similar to those described herein. ¹H NMR (400 MHz, DMSO-d₆,120° C.) δ 7.38-7.16 (m, 9H), 4.65 (s, 2H), 4.23 (s, 2H), 3.04 (t, J=7.2 Hz, 2H), 2.64-2.54 (m, 1H), 2.22 (dt, J=13.4, 7.7 Hz, 1H), 1.14 (d, J=6.7 Hz, 3H), 0.32-0.14 (m, 3H). MS (ESI⁺) m/z 418 (M+H)⁺.

### Example 977

### N-benzyl-N-[(1S)-1-cyclopropylethyl]-2-[(4R)-2,5-dioxo-2′,3′-dihydro-1H-spiro[imidazolidine-4,1′-inden]-1-yl]acetamide

The title compound was prepared using the appropriate starting materials and a procedure similar to those described herein. ¹H NMR (400 MHz, DMSO-d₆,120° C.) δ 8.28 (s, 1H), 7.26 (dddd, J=24.2, 14.5, 8.8, 3.7 Hz, 9H), 4.65 (s, 1H), 4.21 (t, J=13.0 Hz, 2H), 3.57 (s, 1H), 3.04 (t, J=7.2 Hz, 2H), 2.59 (s, 1H), 1.14 (dd, J=7.1, 2.1 Hz, 2H), 1.09-0.92 (m, 2H), 0.24 (dt, J=16.6, 6.1 Hz, 2H). MS (ESI⁺) m/z 418 (M+H)⁺.

### Example 978

### N-benzyl-N-[(1R)-1-cyclopropylethyl]-2-[(4S)-2,5-dioxo-2′,3′-dihydro-1H-spiro[imidazolidine-4,1′-inden]-1-yl]acetamide

The title compound was prepared using the appropriate starting materials and a procedure similar to those described herein. ¹H NMR (400 MHz, DMSO-d₆,120° C.) δ 7.43-7.18 (m, 8H), 4.65 (s, 2H), 4.41-4.17 (m, 2H), 3.04 (t, J=7.2 Hz, 2H), 2.59 (s, 1H), 2.22 (dt, J=13.3, 7.7 Hz, 1H), 1.14 (d, J=6.8 Hz, 3H), 1.09-0.92 (m, 1H), 0.24 (ddd, J=17.2, 9.5, 5.2 Hz, 3H). MS (ESI⁺) m/z 350 (M+H)⁺.

### Example 979

### N-[4-(acetylamino)benzyl]-N-[(1S)-1-cyclobutylethyl]-2-[(4R)-2,5-dioxo-2′,3′-dihydro-1H-spiro[imidazolidine-4,1′-inden]-1-yl]acetamide

The title compound was prepared using the appropriate starting materials and a procedure similar to those described herein.

### Example 980

### N-[(1S)-1-cyclobutylethyl]-2-[(4R)-2,5-dioxo-2′,3′-dihydro-1H-spiro[imidazolidine-4,1′-inden]-1-yl]-N-(4-ethynylbenzyl)acetamide

The title compound was prepared using the appropriate starting materials and a procedure similar to those described herein. ¹H NMR (400 MHz, DMSO-d₆,120° C.) δ 8.31 (s, 1H), 4.59-3.96 (m, 6H), 3.78 (d, J=2.4 Hz, 1H), 3.04 (t, J=7.2 Hz, 3H), 2.65-2.55 (m, 1H), 2.23 (dt, J=13.3, 7.6 Hz, 1H), 2.14-1.62 (m, 7H), 0.99 (d, J=6.6 Hz, 4H). MS (ESI⁺) m/z 456 (M+H)⁺.

### Example 981

### N-[(1S)-1-cyclobutylethyl]-2-[(4R)-2,5-dioxo-2′,3′-dihydro-1H-spiro[imidazolidine-4,1′-inden]-1-yl]-N-({1-[2-(methylamino)-2-oxoethyl]-1H-1,2,3-triazol-4-yl}methyl)acetamide

The title compound was prepared using the appropriate starting materials and a procedure similar to those described herein. ¹H NMR (400 MHz, DMSO-d₆,120° C.) δ 8.31 (s, 1H), 7.52 (d, J=30.2 Hz, 3H), 7.35-7.16 (m, 5H), 4.96 (s, 2H), 4.57-4.28 (m, 5H), 4.13 (s, 1H), 3.04 (t, J=7.2 Hz, 3H), 2.59 (dd, J=24.8, 5.7 Hz, 5H), 2.23 (dt, J=13.3, 7.7 Hz, 1H), 2.00 (s, 1H), 1.93-1.54 (m, 6H), 1.01 (d, J=6.6 Hz, 3H). MS (ESI⁺) m/z 494 (M+H)⁺.

### Example 982

### 2-{4-[([(1S)-1-cyclobutylethyl]{[(4R)-2,5-dioxo-2′,3′-dihydro-1H-spiro[imidazolidine-4,1′-inden]-1-yl]acetyl}amino)methyl]-1H-1,2,3-triazol-1-yl}-N,N-dimethylacetamide

The title compound was prepared using the appropriate starting materials and a procedure similar to those described herein. ¹H NMR (400 MHz, DMSO-d₆,120° C.) δ 8.28 (s, 1H), 7.74 (s, 1H), 7.34-7.16 (m, 4H), 5.27 (s, 2H), 4.62-3.97 (m, 6H), 3.04 (t, J=7.2 Hz, 3H), 2.68-2.54 (m, 2H), 2.23 (dt, J=13.3, 7.7 Hz, 1H), 2.13-1.50 (m, 7H), 1.03 (d, J=6.6 Hz, 4H). MS (ESI⁺) m/z 508 (M+H)⁺.

### Example 983

### N³-[(1S)-1-cyclobutylethyl]-N³-{[(4R)-2,5-dioxo-2′,3′-dihydro-1H-spiro[imidazolidine-4,1′-inden]-1-yl]acetyl}-N-methyl-beta-alaninamide

The title compound was prepared using the appropriate starting materials and a procedure similar to those described herein. ¹H NMR (400 MHz, DMSO-d₆,120° C.) δ 8.27 (s, 1H), 7.51-7.16 (m, 5H), 4.39-4.17 (m, 2H), 4.00 (s, 1H), 3.37 (q, J=8.3 Hz, 3H), 3.04 (t, J=7.2 Hz, 4H), 2.65-2.55 (m, 4H), 2.40-2.16 (m, 3H), 2.13-1.59 (m, 7H), 1.05 (d, J=6.7 Hz, 3H). MS (ESI⁺) m/z 427 (M+H)⁺.

### Example 984

### N-benzyl-N-(1-cyclopropylethyl)-2-[5′-(6-hydroxypyridin-3-yl)-2,5-dioxo-2′,3′-dihydro-1H-spiro[imidazolidine-4,1′-inden]-1-yl]acetamide

The title compound was prepared using the appropriate starting materials and a procedure similar to those described herein. ¹H NMR (400 MHz, DMSO-d₆,120° C.) δ 8.34-8.26 (m, 1H), 7.72 (dd, J=9.5, 2.8 Hz, 1H), 7.59 (d, J=2.8 Hz, 1H), 7.46-7.16 (m, 7H), 6.39 (d, J=9.5 Hz, 1H), 4.65 (s, 2H), 4.23 (d, J=3.5 Hz, 2H), 3.59 (s, 1H), 3.06 (t, J=7.2 Hz, 3H), 2.58 (dt, J=13.3, 6.7 Hz, 4H), 2.25 (dt, J=13.3, 7.7 Hz, 1H), 1.15 (d, J=6.6 Hz, 3H), 0.95 (ddt, J=13.2, 8.1, 4.0 Hz, 1H), 0.55-0.41 (m, 1H), 0.32-0.15 (m, 3H). MS (ESI⁺) m/z 511 (M+H)⁺.

### Example 985

### N-benzyl-N-[(1S)-1-cyclopropylethyl]-2-[5′-(formylamino)-2,5-dioxo-2′,3′-dihydro-1H-spiro[imidazolidine-4,1′-inden]-1-yl]acetamide

The title compound was prepared using the appropriate starting materials and a procedure similar to those described herein. ¹H NMR (400 MHz, DMSO-d₆,120° C.) δ 9.70 (s, 1H), 8.31 (d, J=51.7 Hz, 1H), 7.58-7.11 (m, 7H), 4.65 (s, 2H), 4.22 (s, 2H), 3.59 (s, 1H), 3.00 (q, J=6.7 Hz, 3H), 2.64-2.54 (m, 1H), 2.21 (dtd, J=12.3, 7.7, 4.4 Hz, 1H), 2.01 (s, 1H), 1.14 (d, J=6.8 Hz, 3H), 1.04-0.87 (m, 1H), 0.47 (dtd, J=7.1, 5.3, 4.8, 2.1 Hz, 1H), 0.23 (dtd, J=17.7, 6.0, 3.6 Hz, 3H). MS (ESI⁺) m/z 461 (M+H)⁺.

### Example 986

### N-benzyl-N-[(1S)-1-cyclopropylethyl]-2-[2′,4′-dioxo-5-(2H-tetrazol-5-yl)-2,3-dihydro-3′H-spiro[indene-1,5′-[1,3]oxazolidin]-3′-yl]acetamide

The title compound was prepared using the appropriate starting materials and a procedure similar to those described herein. ¹H NMR (400 MHz, DMSO-d₆,120° C.) δ 8.09-7.91 (m, 1H), 7.64 (d, J=8.0 Hz, 1H), 7.38-7.19 (m, 3H), 4.70 (s, 1H), 4.42 (s, 1H), 3.62 (s, 1H), 3.37-3.11 (m, 4H), 2.85-2.54 (m, 9H), 1.18 (d, J=6.7 Hz, 2H), 1.08-0.80 (m, 1H), 0.27 (ddd, J=14.7, 8.2, 4.0 Hz, 2H). MS (ESI⁺) m/z 419 (M+H)⁺.

### Example 987

### 2,2′-({[3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]carbonyl}imino)diacetic acid

The title compound was prepared using the appropriate starting materials and a procedure similar to those described herein.

### Example 988

### N-benzyl-2-[(4R)-5′-bromo-2,5-dioxo-2′,3′-dihydro-1H-spiro[imidazolidine-4,1′-inden]-1-yl]-N-[(1S)-1-cyclopropylethyl]acetamide

The title compound was prepared using the appropriate starting materials and a procedure similar to those described herein. ¹H NMR (400 MHz, DMSO-d₆,120° C.) δ 7.56-7.20 (m, 8H), 7.19 (s, 1H), 4.65 (s, 2H), 4.23 (s, 2H), 3.04 (t, J=7.2 Hz, 2H), 2.59 (d, J=6.7 Hz, 1H), 2.23 (dt, J=13.4, 7.7 Hz, 1H), 1.14 (d, J=6.7 Hz, 3H), 0.23 (ddd, J=19.8, 9.6, 5.4 Hz, 3H). MS (ESI⁺) m/z 498 (M+H)⁺.

### Example 989

### N-(4-fluorobenzyl)-2-[(1 S,3R)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-[(1S,3R)-5-bromo-3-hydroxy-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.08 (s, 1H), 7.83 (s, 1H), 7.74-7.60 (m, 1H), 7.57 (dd, J=8.1, 1.6 Hz, 1H), 7.48 (s, 1H), 7.40-7.26 (m, 3H), 7.11 (t, J=8.8 Hz, 2H), 5.37 (d, J=5.2 Hz, 1H), 5.18 (dq, J=14.0, 7.7, 6.8 Hz, 2H), 4.87-4.26 (m, 6H), 3.02 (dd, J=14.2, 7.0 Hz, 1H), 2.65 (d, J=4.7 Hz, 3H), 2.32 (dd, J=14.2, 5.2 Hz, 1H), 1.36 (d, J=7.0 Hz, 3H). MS (ESI⁺) m/z 618 (M+H)⁺.

### Example 990

### 2-{(1S,3R)-5-[1-(2-amino-2-oxoethyl)-1H-pyrazol-4-yl]-3-hydroxy-2′,4′-dioxo-2,3-dihydro-3′H-spiro[indene-1,5′-[1,3]oxazolidin]-3′-yl}-N-(4-fluorobenzyl)-N-[(2S)-1,1,1-trifluoropropan-2-yl]acetamide

The title compound was prepared as described in EXAMPLE 295, substituting 2-[(1S,3R)-5-bromo-3-hydroxy-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.09 (s, 1H), 7.83 (s, 1H), 7.64 (d, J=1.5 Hz, 1H), 7.57 (dd, J=8.0, 1.7 Hz, 1H), 7.44-7.24 (m, 3H), 7.11 (t, J=8.8 Hz, 2H), 6.89 (s, 2H), 5.39 (s, 1H), 5.19 (q, J=6.0 Hz, 2H), 4.88-4.26 (m, 6H), 3.02 (dd, J=14.2, 7.0 Hz, 1H), 2.31 (dd, J=14.2, 5.1 Hz, 1H), 1.36 (d, J=7.0 Hz, 3H). MS (ESI⁺) m/z 604 (M+H)⁺.

### Example 991

### N-(4-fluorobenzyl)-2-[(1S)-5-(1-methyl-1H-pyrazol-4-yl)-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-[(1S)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.00 (s, 1H), 7.76 (d, J=0.8 Hz, 1H), 7.52 (dd, J=1.7, 0.9 Hz, 1H), 7.45 (dd, J=8.0, 1.6 Hz, 1H), 7.42-7.20 (m, 3H), 7.19-7.00 (m, 2H), 5.24-5.09 (m, 1H), 4.86-4.28 (m, 4H), 3.84 (s, 3H), 3.27-2.95 (m, 2H), 2.77-2.46 (m, 2H), 1.37 (d, J=7.0 Hz, 3H). MS (ESI⁺) m/z 545 (M+H)⁺.

### Example 992

### 2-[1,1-dioxido-2′,5′-dioxo-6-(pyridin-3-yl)-1′H-spiro[1-benzothiophene-3,4′-imidazolidin]-1′-yl]-N-(4-fluorobenzyl)-N-[(2S)-1,1,1-trifluoropropan-2-yl]acetamide

The title compound was prepared as described in EXAMPLE 295, substituting 2-(6-bromo-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and pyridin-3-ylboronic acid for 5-(4,4,5,5-tetramethyl-1,3,2-dioxaborolan-2-yl)pyridin-2-ol. ¹H NMR (400 MHz, DMSO-d₆, 120° C.) δ ppm 9.06-8.92 (m, 2H), 8.63 (dd, J=4.8, 1.6 Hz, 1H), 8.24-8.08 (m, 3H), 7.84-7.74 (m, 1H), 7.50 (dd, J=7.9, 4.8 Hz, 1H), 7.38-7.26 (m, 2H), 7.19-7.03 (m, 2H), 5.28-5.10 (m, 1H), 4.82 (dd, J=17.7, 5.8 Hz, 1H), 4.70-4.45 (m, 2H), 4.30 (dd, J=16.7, 5.3 Hz, 1H), 4.01 (dd, J=14.4, 4.0 Hz, 1H), 3.84 (dd, J=14.4, 4.1 Hz, 1H), 1.35 (d, J=7.1, 2.2 Hz, 3H). MS (ESI⁺) m/z 591 (M+H)⁺.

### Example 993

### 2-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]benzoic acid

### Example 993A

### (R)-methyl 2-(N-(4-fluorobenzyl)-2-(5-(3-methylureido)-2′,4′-dioxo-2,3-dihydrospiro[indene-1,5′-oxazolidin]-3′-yl)acetamido)benzoate

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and methyl 2-((4-fluorobenzyl)amino)benzoate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 993B

### 2-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]benzoic acid

A solution of (R)-methyl 2-(N-(4-fluorobenzyl)-2-(5-(3-methylureido)-2′,4′-dioxo-2,3-dihydrospiro[indene-1,5′-oxazolidin]-3′-yl)acetamido)benzoate (0.025 g, 0.044 mmol) in 1,2-dichloroethane (0.870 ml) was treated with trimethylstannanol (0.071 g, 0.392 mmol) and the reaction mixture was heated at 75° C. for 30 hours. The reaction mixture was cooled to room temperature and concentrated. The residue was taken up in ethyl acetate and washed with aqueous 1N HCl (twice) and brine. The organic layer was dried over anhydrous sodium sulfate, filtered, and concentrated. The concentrate was purified by preparative reverse-phase HPLC to give the title compound. ¹H NMR (400 MHz, DMSO-d₆, mixture of rotamers) δ ppm 13.36 (brs, 1H), 8.70 (d, J=3.8 Hz, 1H), 8.00 (dd, J=7.7, 1.7 Hz, 1H), 7.68-7.59 (m, 1H), 7.59-7.51 (m, 2H), 7.33-7.25 (m, 1H), 7.26-7.15 (m, 3H), 7.15-7.03 (m, 3H), 6.16-6.00 (m, 1H), 5.26 (dd, J=14.8, 11.4 Hz, 1H), 4.30 (dd, J=14.8, 8.7 Hz, 1H), 4.00 (t, J=16.7 Hz, 1H), 3.87 (dd, J=23.8, 16.6 Hz, 1H), 3.14-3.06 (m, 1H), 3.03-2.92 (m, 1H), 2.69-2.56 (m, 4H), 2.47-2.39 (m, 1H). MS (ESI⁺) m/z 561 (M+H)⁺.

### Example 994

### methyl 3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]butanoate

The title compound was prepared as described in EXAMPLE 306, substituting (R)-2-(5-(3-methylureido)-2′,4′-dioxo-2,3-dihydrospiro[indene-1,5′-oxazolidin]-3′-yl)acetic acid for cyclopropanecarboxylic acid and methyl 3-((4-fluorobenzyl)amino)butanoate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide. ¹H NMR (400 MHz, DMSO-d₆, 120° C.) δ ppm 8.24 (brs, 1H), 7.48 (brs, 1H), 7.42-7.17 (m, 4H), 7.14-7.01 (m, 2H), 5.93-5.77 (m, 1H), 4.73-4.35 (m, 5H), 3.55 (s, 3H), 3.19-3.06 (m, 1H), 3.05-2.92 (m, 1H), 2.72-2.59 (m, 4H), 2.54 (dd, J=15.9, 6.6 Hz, 1H), 2.49-2.41 (m, 2H), 1.23-1.13 (m, 3H). MS (APCI⁺) m/z 541 (M+H)⁺.

### Example 995

### 3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]butanoic acid

A solution of methyl 3-[(4-fluorobenzyl)({(1R)-5-[(methylcarbamoyl)amino]-2′,4′-dioxo-2,3-dihydro-3′H-spiro[indene-1,5′-[1,3]oxazolidin]-3′-yl}acetyl)amino]butanoate (0.07 g, 0.130 mmol) in 1,2-dichloroethane (2.158 ml) was treated with trimethylstannanol (0.187 g, 1.036 mmol) and the reaction mixture was heated at 75° C. for 1 hour. The reaction mixture was cooled to room temperature and concentrated. The residue was taken up in ethyl acetate and washed with aqueous 1N HCl (twice) and brine. The organic layer was dried over anhydrous sodium sulfate, filtered, and concentrated. The concentrate was purified by silica gel column chromatography to afford the title compound. ¹H NMR (400 MHz, DMSO-d₆, mixture of rotamers) δ ppm 12.33 (brs, 1H), 8.75-8.64 (m, 1H), 7.55 (s, 1H), 7.44-7.34 (m, 1H), 7.34-7.13 (m, 4H), 7.14-7.02 (m, 1H), 6.12-6.01 (m, 1H), 4.85 (t, J=17.0 Hz, 1H), 4.76-4.24 (m, 5H), 3.19-3.05 (m, 1H), 3.05-2.93 (m, 1H), 2.71-2.54 (m, 4H), 2.53-2.42 (m, 2H), 1.20-1.06 (m, 3H). MS (APCI⁺) m/z 527 (M+H)⁺.

### Example 996

### 2-{(1S)-5-[1-(2-amino-2-oxoethyl)-1H-pyrazol-4-yl]-2′,4′-dioxo-2,3-dihydro-3′H-spiro[indene-1,5′-[1,3]oxazolidin]-3′-yl}-N-(4-fluorobenzyl)-N-[(2S)-1,1,1-trifluoropropan-2-yl]acetamide

The title compound was prepared as described in EXAMPLE 295, substituting 2-[(1S)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.07 (s, 1H), 7.82 (s, 1H), 7.60-7.44 (m, 2H), 7.43-7.25 (m, 3H), 7.12 (t, J=8.8 Hz, 2H), 6.87 (s, 2H), 5.27-5.05 (m, 1H), 4.89-4.27 (m, 6H), 3.39-3.01 (m, 2H), 2.78-2.47 (m, 2H), 1.37 (d, J=7.0 Hz, 3H). MS (ESI⁺) m/z 588 (M+H)⁺.

### Example 997

### N-(4-fluorobenzyl)-2-[(3′S,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide

The title compound was isolated as a byproduct of EXAMPLE 715. ¹H NMR (400 MHz, DMSO-d₆) δ 8.30 (d, J=2.7 Hz, 1H), 8.10 (s, 1H), 7.85 (s, 1H), 7.76-7.38 (m, 3H), 7.38-7.05 (m, 5H), 6.13 (dd, J=6.4, 3.7 Hz, 1H), 5.15 (dt, J=12.0, 6.8 Hz, 1H), 4.88-4.72 (m, 3H), 4.72-4.32 (m, 2H), 4.26 (d, J=16.5 Hz, 1H), 4.04 (q, J=7.1 Hz, 1H), 2.76-2.53 (m, 5H), 1.95 (s, 1H), 1.79 (s, 1H), 1.34 (d, J=7.1 Hz, 3H), 1.29-1.14 (m, 1H). MS (ESI⁺) m/z 619 (M+H)⁺.

### Example 998

### N-(4-fluorobenzyl)-2-[(1R,3R)-3-hydroxy-5-(1-methyl-1H-pyrazol-4-yl)-2′,4′-dioxo-2,3-dihydro-3′H-spiro[indene-1,5′-[1,3]oxazolidin]-3′-yl]-N-[(2S)-1,1,1-trifluoropropan-2-yl]acetamide

### Example 998A

### (R)-5-bromospiro[indene-1,5′-oxazolidine]-2′,3,4′(2H)-trione

The title compound was prepared as described in EXAMPLE 685A, substituting (R)-5′-bromo-2′,3′-dihydrospiro[imidazolidine-4,1′-indene]-2,5-dione for (S)-5′-bromo-2′,3′-dihydrospiro[imidazolidine-4,1′-indene]-2,5-dione.

### Example 998B

### (1R,3R)-5-bromo-3-hydroxy-2,3-dihydrospiro[indene-1,5′-oxazolidine]-2′,4′-dione

(R)-5-Bromospiro[indene-1,5′-oxazolidine]-2′,3,4′(2H)-trione (1.2 g, 2.027 mmol) in 4 mL tetrahydrofuran was cooled to −78° C. and lithium tri-sec-butylborohydride (4.05 ml, 4.05 mmol) was added dropwise. The mixture was warmed from −78° C. to 0° C., added to water and adjusted to pH 3-4 with 2N HCl. The mixture was extracted with ethyl acetate and the organic layer was washed with brine, dried over sodium sulfate, filtered, and concentrated. Silica gel column chromatography afforded the title compound.

### Example 998C

### 2-((1R,3R)-5-bromo-3-hydroxy-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide

The title compound was prepared as described in EXAMPLE 281F, substituting (1R,3R)-5-bromo-3-hydroxy-2,3-dihydrospiro[indene-1,5′-oxazolidine]-2′,4′-dione for (R)-5-bromo-2,3-dihydrospiro[indene-1,5′-oxazolidine]-2′,4′-dione.

### Example 998D

### N-(4-fluorobenzyl)-2-[(1R,3R)-3-hydroxy-5-(1-methyl-1H-pyrazol-4-yl)-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-((1R,3R)-5-bromo-3-hydroxy-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.02 (s, 1H), 7.77 (s, 1H), 7.61 (t, J=1.1 Hz, 1H), 7.53 (dd, J=8.0, 1.6 Hz, 1H), 7.43-7.28 (m, 3H), 7.13 (t, J=8.8 Hz, 2H), 5.44-5.28 (m, 2H), 5.24-5.10 (m, 1H), 4.84 (d, J=17.6 Hz, 1H), 4.63 (d, J=17.8 Hz, 2H), 4.40 (d, J=16.8 Hz, 1H), 3.85 (s, 3H), 2.80 (s, 1H), 2.50 (m, 1H), 1.38 (d, J=7.1 Hz, 3H). MS (ESI⁺) m/z 561 (M+H)⁺.

### Example 999

### N-(4-fluorobenzyl)-2-[(1R,3R)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-((1R,3R)-5-bromo-3-hydroxy-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.09 (s, 1H), 7.83 (s, 1H), 7.74-7.59 (m, 1H), 7.55 (dd, J=8.1, 1.6 Hz, 2H), 7.43-7.27 (m, 3H), 7.17-7.00 (m, 2H), 5.48-5.26 (m, 2H), 5.20 (m, 1H), 4.94-4.30 (m, 6H), 2.81 (s, 1H), 2.65 (d, J=4.7 Hz, 3H), 2.42 (d, J=6.4 Hz, 1H), 1.38 (d, J=7.0 Hz, 3H). MS (ESI⁺) m/z 618 (M+H)⁺.

### Example 1000

### N-(4-fluorobenzyl)-2-[(1 S,3S)-3-hydroxy-5-(1-methyl-1H-pyrazol-4-yl)-2′,4′-dioxo-2,3-dihydro-3′H-spiro[indene-1,5′-[1,3]oxazolidin]-3′-yl]-N-[(2S)-1,1,1-trifluoropropan-2-yl]acetamide

### Example 1000A

### (1S,3S)-5-bromo-3-hydroxy-2,3-dihydrospiro[indene-1,5′-oxazolidine]-2′,4′-dione

The title compound was prepared as described in EXAMPLE 998B, substituting (S)-5-bromospiro[indene-1,5′-oxazolidine]-2′,3,4′(2H)-trione for (R)-5-bromospiro[indene-1,5′-oxazolidine]-2′,3,4′(2H)-trione.

### Example 1000B

### 2-((1 S,3S)-5-bromo-3-hydroxy-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide

The title compound was prepared as described in EXAMPLE 281F, substituting (1S,3S)-5-bromo-3-hydroxy-2,3-dihydrospiro[indene-1,5′-oxazolidine]-2′,4′-dione for (R)-5-bromo-2,3-dihydrospiro[indene-1,5′-oxazolidine]-2′,4′-dione.

### Example 1000C

### N-(4-fluorobenzyl)-2-[(1 S,3S)-3-hydroxy-5-(1-methyl-1H-pyrazol-4-yl)-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-((1S,3S)-5-bromo-3-hydroxy-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.02 (s, 1H), 7.77 (d, J=0.9 Hz, 1H), 7.72-7.57 (m, 1H), 7.59-7.47 (m, 1H), 7.44-7.26 (m, 3H), 7.12 (t, J=8.8 Hz, 2H), 5.43-5.28 (m, 2H), 5.25-5.09 (m, 1H), 4.90-4.22 (m, 4H), 3.85 (s, 3H), 2.97-2.83 (m, 1H), 2.46-2.33 (m, 1H), 1.38 (d, J=7.1 Hz, 3H). MS (ESI⁺) m/z 561 (M+H)⁺.

### Example 1001

### 2-[(1S)-5-{1-[2-(dimethylamino)-2-oxoethyl]-1H-pyrazol-4-yl}-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 295, substituting 2-[(1S)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N,N-dimethyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.02 (s, 1H), 7.77 (s, 1H), 7.64-7.57 (m, 1H), 7.53 (dd, J=8.0, 1.6 Hz, 1H), 7.43-7.25 (m, 3H), 7.12 (t, J=8.8 Hz, 2H), 5.44-5.11 (m, 3H), 4.96-4.26 (m, 4H), 3.85 (s, 3H), 2.80 (m, 1H), 2.46-2.36 (m, 1H), 1.38 (d, J=7.0 Hz, 3H). MS (ESI⁺) m/z 616 (M+H)⁺.

### Example 1002

### 2-[(1S,3R)-5-bromo-3-fluoro-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 887, substituting 2-((1S,3S)-5-bromo-3-hydroxy-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for N-(4-fluorobenzyl)-2-[(1 S,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide. ¹H NMR (400 MHz, DMSO-d₆) δ 7.85 (s, 1H), 7.75 (dt, J=8.3, 1.9 Hz, 1H), 7.50 (dd, J=8.3, 1.3 Hz, 1H), 7.32 (dd, J=8.5, 5.3 Hz, 2H), 7.12 (t, J=8.8 Hz, 2H), 6.11 (ddd, J=55.5, 6.3, 2.5 Hz, 1H), 5.17 (m, 1H), 4.93-4.22 (m, 4H), 3.15 (ddd, J=20.7, 15.8, 6.4 Hz, 1H), 2.66 (ddd, J=21.9, 15.8, 2.5 Hz, 1H), 1.37 (d, J=7.0 Hz, 3H). MS (ESI⁺) m/z 562 (M+H)⁺.

### Example 1003

### 2-[(1R)-2′,4′-dioxo-5-(pyridin-4-yl)-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 295, substituting 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 4-(4,4,5,5-tetramethyl-1,3,2-dioxaborolan-2-yl)pyridine for 5-(4,4,5,5-tetramethyl-1,3,2-dioxaborolan-2-yl)pyridin-2-ol. ¹H NMR (400 MHz, DMSO-d₆, 120° C.) δ ppm 8.71-8.61 (m, 2H), 7.83-7.63 (m, 4H), 7.56 (d, J=8.0 Hz, 1H), 7.40-7.20 (m, 2H), 7.20-7.07 (m, 2H), 5.24-5.11 (m, 1H), 4.84 (d, J=17.6 Hz, 1H), 4.63 (d, J=17.2 Hz, 2H), 4.40 (d, J=16.8 Hz, 1H), 3.35-3.16 (m, 2H), 2.84-2.69 (m, 1H), 2.61-2.53 (m, 1H), 1.37 (d, J=7.0 Hz, 3H). MS (ESI⁺) m/z 542 (M+H)⁺.

### Example 1004

### 2-[(1S)-2′,4′-dioxo-5-(pyridin-4-yl)-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 295, substituting 2-[(1S)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 4-(4,4,5,5-tetramethyl-1,3,2-dioxaborolan-2-yl)pyridine for 5-(4,4,5,5-tetramethyl-1,3,2-dioxaborolan-2-yl)pyridin-2-ol. ¹H NMR (400 MHz, DMSO-d₆, 120° C.) δ ppm 8.72-8.62 (m, 2H), 7.84-7.67 (m, 4H), 7.56 (d, J=8.1 Hz, 1H), 7.33 (dd, J=8.4, 5.3 Hz, 2H), 7.12 (t, J=8.8 Hz, 2H), 5.31-5.11 (m, 1H), 4.83 (d, J=17.6 Hz, 1H), 4.71-4.58 (m, 2H), 4.38 (d, J=16.8 Hz, 1H), 3.27-3.06 (m, 2H), 2.75 (ddd, J=14.7, 8.5, 6.2 Hz, 1H), 2.57 (ddd, J=14.5, 8.4, 4.7 Hz, 1H), 1.37 (d, J=7.0 Hz, 3H). MS (ESI⁺) m/z 542 (M+H)⁺.

### Example 1005

### 2-[(1R)-2′,4′-dioxo-5-(pyridin-3-yl)-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 295, substituting 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 3-(4,4,5,5-tetramethyl-1,3,2-dioxaborolan-2-yl)pyridine for 5-(4,4,5,5-tetramethyl-1,3,2-dioxaborolan-2-yl)pyridin-2-ol. ¹H NMR (400 MHz, DMSO-d₆, 120° C.) δ ppm 8.86 (d, J=2.3 Hz, 1H), 8.57 (dd, J=4.9, 1.5 Hz, 1H), 8.12-8.02 (m, 1H), 7.75-7.43 (m, 4H), 7.33 (dd, J=8.4, 5.3 Hz, 2H), 7.12 (t, J=8.9 Hz, 2H), 5.29-5.13 (m, 1H), 4.84 (d, J=17.6 Hz, 1H), 4.63 (d, J=17.4 Hz, 2H), 4.40 (d, J=16.8 Hz, 1H), 3.22-3.08 (m, 2H), 2.83-2.68 (m, 1H), 2.62-2.54 (m, 1H), 1.37 (d, J=7.0 Hz, 3H). MS (ESI⁺) m/z 542 (M+H)⁺.

### Example 1006

### 2-[(1S)-2′,4′-dioxo-5-(pyridin-3-yl)-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 295, substituting 2-[(1S)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 3-(4,4,5,5-tetramethyl-1,3,2-dioxaborolan-2-yl)pyridine for 5-(4,4,5,5-tetramethyl-1,3,2-dioxaborolan-2-yl)pyridin-2-ol. ¹H NMR (400 MHz, DMSO-d₆, 120° C.) δ ppm 8.93-8.85 (m, 1H), 8.64-8.54 (m, 1H), 8.15-8.04 (m, 1H), 7.70 (s, 1H), 7.67-7.58 (m, 1H), 7.58-7.45 (m, 2H), 7.33 (dd, J=8.5, 5.4 Hz, 2H), 7.12 (t, J=8.8 Hz, 2H), 5.24-5.13 (m, 1H), 4.83 (d, J=17.5 Hz, 1H), 4.76-4.45 (m, 2H), 4.38 (d, J=16.8 Hz, 1H), 3.32-3.09 (m, 2H), 2.84-2.67 (m, 1H), 2.67-2.51 (m, 1H), 1.38 (d, J=7.1 Hz, 3H). MS (ESI⁺) m/z 542 (M+H)⁺.

### Example 1007

### 2-[(1R)-2′,4′-dioxo-5-(6-oxo-1,6-dihydropyridin-3-yl)-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 295, substituting 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 5-(4,4,5,5-tetramethyl-1,3,2-dioxaborolan-2-yl)pyridin-2(1H)-one for 5-(4,4,5,5-tetramethyl-1,3,2-dioxaborolan-2-yl)pyridin-2-ol. ¹H NMR (400 MHz, DMSO-d₆, 120° C.) δ ppm 7.74 (dd, J=9.5, 2.8 Hz, 1H), 7.67-7.62 (m, 1H), 7.51 (s, 1H), 7.48-7.37 (m, 2H), 7.33 (dd, J=8.5, 5.4 Hz, 2H), 7.12 (t, J=8.8 Hz, 2H), 6.41 (d, J=9.5 Hz, 1H), 5.28-5.11 (m, 1H), 4.83 (d, J=17.6 Hz, 1H), 4.62 (d, J=18.4 Hz, 2H), 4.51-4.24 (m, 1H), 3.25-3.05 (m, 2H), 2.76-2.65 (m, 1H), 2.57-2.49 (m, 1H), 1.37 (d, J=7.1 Hz, 3H). MS (APCI⁺) m/z 558 (M+H)⁺.

### Example 1008

### 2-[(1S)-2′,4′-dioxo-5-(6-oxo-1,6-dihydropyridin-3-yl)-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 295, substituting 2-[(1S)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 5-(4,4,5,5-tetramethyl-1,3,2-dioxaborolan-2-yl)pyridin-2(1H)-one for 5-(4,4,5,5-tetramethyl-1,3,2-dioxaborolan-2-yl)pyridin-2-ol. ¹H NMR (400 MHz, DMSO-d₆, 120° C.) δ ppm 7.74 (dd, J=9.5, 2.8 Hz, 1H), 7.71-7.62 (m, 1H), 7.51 (s, 1H), 7.47-7.39 (m, 2H), 7.33 (dd, J=8.4, 5.3 Hz, 2H), 7.12 (t, J=8.8 Hz, 2H), 6.40 (d, J=9.4 Hz, 1H), 5.28-5.06 (m, 1H), 4.82 (d, J=17.6 Hz, 1H), 4.78-4.57 (m, 2H), 4.36 (d, J=16.8 Hz, 1H), 3.28-3.04 (m, 2H), 2.75-2.66 (m, 1H), 2.57-2.48 (m, 1H), 1.37 (d, J=7.0 Hz, 3H). MS (APCI⁺) m/z 558 (M+H)⁺.

### Example 1009

### 2-[(1R)-2′,4′-dioxo-5-phenyl-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 295, substituting 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and phenylboronic acid for 5-(4,4,5,5-tetramethyl-1,3,2-dioxaborolan-2-yl)pyridin-2-ol. ¹H NMR (400 MHz, DMSO-d₆, 120° C.) δ ppm 7.68-7.59 (m, 3H), 7.59-7.52 (m, 1H), 7.52-7.40 (m, 3H), 7.40-7.30 (m, 3H), 7.12 (t, J=8.8 Hz, 2H), 5.24-5.13 (m, 1H), 4.84 (d, J=17.5 Hz, 1H), 4.62 (d, J=17.6 Hz, 2H), 4.40 (d, J=16.8 Hz, 1H), 3.33-3.06 (m, 2H), 2.77-2.68 (m, 1H), 2.59-2.49 (m, 1H), 1.37 (d, J=7.0 Hz, 3H). MS (ESI⁺) m/z 541 (M+H)⁺.

### Example 1010

### 2-[(1S)-2′,4′-dioxo-5-phenyl-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 295, substituting 2-[(1S)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and phenylboronic acid for 5-(4,4,5,5-tetramethyl-1,3,2-dioxaborolan-2-yl)pyridin-2-ol. ¹H NMR (400 MHz, DMSO-d₆, 120° C.) δ ppm 7.69-7.59 (m, 3H), 7.56 (dd, J=8.1, 1.6 Hz, 1H), 7.52-7.40 (m, 3H), 7.40-7.29 (m, 3H), 7.12 (t, J=8.8 Hz, 2H), 5.24-5.11 (m, 1H), 4.83 (d, J=17.6 Hz, 1H), 4.77-4.54 (m, 2H), 4.38 (d, J=16.9 Hz, 1H), 3.33-3.06 (m, 2H), 2.79-2.66 (m, 1H), 2.63-2.50 (m, 1H), 1.37 (d, J=7.0 Hz, 3H). MS (ESI⁺) m/z 541 (M+H)⁺.

### Example 1011

### N-cyclohexyl-N-(4-fluorobenzyl)-2-[(1R)-5-(1-methyl-1H-pyrazol-4-yl)-2′,4′-dioxo-2,3-dihydro-3′H-spiro[indene-1,5′-[1,3]oxazolidin]-3′-yl]acetamide

### Example 1011A

### N-(4-fluorobenzyl)cyclohexanamine

The title compound was prepared as described in EXAMPLE 283A, substituting cyclohexanamine for (S)-1-cyclopropylethanamine and 4-fluorobenzaldehyde for benzaldehyde.

### Example 1011B

### (R)-2-(5-bromo-2′,4′-dioxo-2,3-dihydrospiro[indene-1,5′-oxazolidin]-3′-yl)-N-cyclohexyl-N-(4-fluorobenzyl)acetamide

The title compound was prepared as described in EXAMPLE 306, substituting (R)-2-(5-bromo-2′,4′-dioxo-2,3-dihydrospiro[indene-1,5′-oxazolidin]-3′-yl)acetic acid for cyclopropanecarboxylic acid and N-(4-fluorobenzyl)cyclohexanamine for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide

### Example 1011C

### N-cyclohexyl-N-(4-fluorobenzyl)-2-[(1R)-5-(1-methyl-1H-pyrazol-4-yl)-2′,4′-dioxo-2,3-dihydro-3′H-spiro[indene-1,5′-[1,3]oxazolidin]-3′-yl]acetamide

The title compound was prepared as described in EXAMPLE 295, substituting (R)-2-(5-bromo-2′,4′-dioxo-2,3-dihydrospiro[indene-1,5′-oxazolidin]-3′-yl)-N-cyclohexyl-N-(4-fluorobenzyl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 7.99 (s, 1H), 7.76 (s, 1H), 7.55-7.36 (m, 3H), 7.30 (dd, J=8.4, 5.4 Hz, 2H), 7.08 (t, J=8.7 Hz, 2H), 4.58 (s, 2H), 4.54-4.40 (m, 2H), 3.85 (s, 3H), 3.28-2.96 (m, 2H), 2.70 (ddd, J=14.9, 8.6, 6.4 Hz, 1H), 2.58-2.45 (m, 1H), 1.82-0.74 (m, 10H). LC-MS (APCI): 531 (M+H)⁺.

### Example 1012

### N-(4-fluorobenzyl)-2-[(1R)-5-(1-methyl-1H-pyrazol-4-yl)-2′,4′-dioxo-2,3-dihydro-3′H-spiro[indene-1,5′-[1,3]oxazolidin]-3′-yl]-N-(tetrahydro-2H-pyran-4-yl)acetamide

### Example 1012A

### (R)-2-(5-bromo-2′,4′-dioxo-2,3-dihydrospiro[indene-1,5′-oxazolidin]-3′-yl)-N-(4-fluorobenzyl)-N-(tetrahydro-2H-pyran-4-yl)acetamide

The title compound was prepared as described in EXAMPLE 306, substituting (R)-2-(5-bromo-2′,4′-dioxo-2,3-dihydrospiro[indene-1,5′-oxazolidin]-3′-yl)acetic acid for cyclopropanecarboxylic acid and N-(4-fluorobenzyl)tetrahydro-2H-pyran-4-amine for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 1012B

### N-(4-fluorobenzyl)-2-[(1R)-5-(1-methyl-1H-pyrazol-4-yl)-2′,4′-dioxo-2,3-dihydro-3′H-spiro[indene-1,5′-[1,3]oxazolidin]-3′-yl]-N-(tetrahydro-2H-pyran-4-yl)acetamide

The title compound was prepared as described in EXAMPLE 295, substituting (R)-2-(5-bromo-2′,4′-dioxo-2,3-dihydrospiro[indene-1,5′-oxazolidin]-3′-yl)-N-(4-fluorobenzyl)-N-(tetrahydro-2H-pyran-4-yl)acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (501 MHz, DMSO-d₆) δ 8.16 (d, J=8.8 Hz, 1H), 7.89 (dd, J=9.1, 0.8 Hz, 1H), 7.62-7.32 (m, 5H), 7.29-7.17 (m, 2H), 7.11 (t, J=8.9 Hz, 1H), 4.82-4.29 (m, 4H), 3.89-3.77 (m, 5H), 3.44-3.26 (m, 1H), 3.25-2.98 (m, 2H), 2.77-2.61 (m, 1H), 2.61-2.49 (m, 1H), 1.67 (dq, J=12.0, 6.0 Hz, 2H), 1.50 (ddd, J=49.0, 12.9, 3.8 Hz, 2H), 0.91-0.77 (m, 1H). LC-MS (APCI): 533 (M+H)⁺.

### Example 1013

### 3-[(4-fluorobenzyl){[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]cyclobutanecarboxylic acid

### Example 1013A

### (R)-tert-butyl 3-(2-(5-bromo-2′,4′-dioxo-2,3-dihydrospiro[indene-1,5′-oxazolidin]-3′-yl)-N-(4-fluorobenzyl)acetamido)cyclobutanecarboxylate

The title compound was prepared as described in EXAMPLE 306, substituting (R)-2-(5-bromo-2′,4′-dioxo-2,3-dihydrospiro[indene-1,5′-oxazolidin]-3′-yl)acetic acid for cyclopropanecarboxylic acid and tert-butyl 3-((4-fluorobenzyl)amino)cyclobutanecarboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 1013B

### (R)-tert-butyl 3-(N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamido)cyclobutanecarboxylate

The title compound was prepared as described in EXAMPLE 295, substituting (R)-tert-butyl 3-(2-(5-bromo-2′,4′-dioxo-2,3-dihydrospiro[indene-1,5′-oxazolidin]-3′-yl)-N-(4-fluorobenzyl)acetamido)cyclobutanecarboxylate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol.

### Example 1013C

### 3-[(4-fluorobenzyl) {[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]cyclobutanecarboxylic acid

The title compound was prepared as described in EXAMPLE 302, substituting (R)-tert-butyl 3-(N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamido)cyclobutanecarboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 8.14 (d, J=4.6 Hz, 1H), 7.86 (d, J=5.3 Hz, 1H), 7.61-7.34 (m, 3H), 7.30-7.04 (m, 4H), 4.86-4.21 (m, 4H), 3.82 (d, J=2.6 Hz, 3H), 3.27-2.94 (m, 2H), 2.72 (ddd, J=72.2, 10.7, 5.5 Hz, 2H), 2.57-2.30 (m, 5H), 2.21 (td, J=9.1, 8.6, 4.4 Hz, 1H). LC-MS (APCI): 537 (M+H)⁺.

### Example 1014

### 4-[(4-fluorobenzyl){[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]cyclohexanecarboxylic acid

### Example 1014A

### (R)-tert-butyl 4-(2-(5-bromo-2′,4′-dioxo-2,3-dihydrospiro[indene-1,5′-oxazolidin]-3′-yl)-N-(4-fluorobenzyl)acetamido)cyclohexanecarboxylate

The title compound was prepared as described in EXAMPLE 306, substituting (R)-2-(5-bromo-2′,4′-dioxo-2,3-dihydrospiro[indene-1,5′-oxazolidin]-3′-yl)acetic acid for cyclopropanecarboxylic acid and tert-butyl 4-((4-fluorobenzyl)amino)cyclohexanecarboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 1014B

### (R)-tert-butyl 4-(N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamido)cyclohexanecarboxylate

The title compound was prepared as described in EXAMPLE 295, substituting (R)-tert-butyl 4-(2-(5-bromo-2′,4′-dioxo-2,3-dihydrospiro[indene-1,5′-oxazolidin]-3′-yl)-N-(4-fluorobenzyl)acetamido)cyclohexanecarboxylate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol.

### Example 1014C

### 4-[(4-fluorobenzyl){[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]cyclohexanecarboxylic acid

The title compound was prepared as described in EXAMPLE 302, substituting (R)-tert-butyl 4-(N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamido)cyclohexanecarboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 8.14 (d, J=6.9 Hz, 1H), 7.86 (d, J=6.4 Hz, 1H), 7.61-7.39 (m, 3H), 7.33 (dd, J=9.5, 4.3 Hz, 1H), 7.27-7.02 (m, 3H), 4.81-4.26 (m, 4H), 3.83 (d, J=4.4 Hz, 3H), 3.28-2.99 (m, 2H), 2.80-2.39 (m, 2H), 2.23-1.79 (m, 2H), 1.71-1.39 (m, 5H), 1.40-1.01 (m, 3H). LC-MS (APCI): 575 (M+H)⁺.

### Example 1015

### N-(4-fluorobenzyl)-2-[(1R)-5-(1-methyl-1H-pyrazol-4-yl)-2′,4′-dioxo-2,3-dihydro-3′H-spiro[indene-1,5′-[1,3]oxazolidin]-3′-yl]-N-{trans-4-[(methylsulfonyl)amino]cyclohexyl}acetamide

### Example 1015A

### tert-butyl {trans-4-[{[(1R)-5-bromo-2′,4′-dioxo-2,3-dihydro-3′H-spiro[indene-1,5′-[1,3]oxazolidin]-3′-yl]acetyl}(4-fluorobenzyl)amino]cyclohexyl}carbamate

The title compound was prepared as described in EXAMPLE 306, substituting (R)-2-(5-bromo-2′,4′-dioxo-2,3-dihydrospiro[indene-1,5′-oxazolidin]-3′-yl)acetic acid for cyclopropanecarboxylic acid and tert-butyl (trans-4-((4-fluorobenzyl)amino)cyclohexyl)carbamate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 1015B

### tert-butyl {trans-4-[(4-fluorobenzyl) {[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]cyclohexyl}carbamate

The title compound was prepared as described in EXAMPLE 295, substituting tert-butyl {trans-4-[{[(1R)-5-bromo-2′,4′-dioxo-2,3-dihydro-3′H-spiro[indene-1,5′-[1,3]oxazolidin]-3′-yl]acetyl}(4-fluorobenzyl)amino]cyclohexyl}carbamate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol.

### Example 1015C

### N-(trans-4-aminocyclohexyl)-N-(4-fluorobenzyl)-2-[(1R)-5-(1-methyl-1H-pyrazol-4-yl)-2′,4′-dioxo-2,3-dihydro-3′H-spiro[indene-1,5′-[1,3]oxazolidin]-3′-yl]acetamide

The title compound was prepared as described in EXAMPLE 302, substituting tert-butyl {trans-4-[(4-fluorobenzyl) {[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]cyclohexyl}carbamate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 1015D

### N-(4-fluorobenzyl)-2-[(1R)-5-(1-methyl-1H-pyrazol-4-yl)-2′,4′-dioxo-2,3-dihydro-3′H-spiro[indene-1,5′-[1,3]oxazolidin]-3′-yl]-N-{trans-4-[(methylsulfonyl)amino]cyclohexyl}acetamide

The title compound was prepared as described in EXAMPLE 308, substituting N-(trans-4-aminocyclohexyl)-N-(4-fluorobenzyl)-2-[(1R)-5-(1-methyl-1H-pyrazol-4-yl)-2′,4′-dioxo-2,3-dihydro-3′H-spiro[indene-1,5′-[1,3]oxazolidin]-3′-yl]acetamide for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide hydrochloride. ¹H NMR (400 MHz, DMSO-d₆) δ 8.14 (d, J=7.2 Hz, 1H), 7.87 (dd, J=7.2, 0.8 Hz, 1H), 7.62-7.39 (m, 3H), 7.33 (dd, J=8.5, 5.4 Hz, 1H), 7.26-7.15 (m, 2H), 7.08 (t, J=8.9 Hz, 1H), 6.98 (dd, J=16.9, 7.5 Hz, 1H), 4.80-4.12 (m, 5H), 3.83 (d, J=4.7 Hz, 3H), 3.23-2.95 (m, 3H), 2.84 (d, J=3.7 Hz, 3H), 2.76-2.47 (m, 2H), 1.93-1.80 (m, 2H), 1.68-1.46 (m, 4H), 1.46-1.17 (m, 2H). LC-MS (APCI): 624 (M+H)⁺.

### Example 1016

### N-(4-fluorobenzyl)-2-[(1R)-5-(1-methyl-1H-pyrazol-4-yl)-2′,4′-dioxo-2,3-dihydro-3′H-spiro[indene-1,5′-[1,3]oxazolidin]-3′-yl]-N-(3-{[(trifluoromethyl)sulfonyl]amino}cyclobutyl)acetamide

### Example 1016A

### (R)-tert-butyl (3-(2-(5-bromo-2′,4′-dioxo-2,3-dihydrospiro[indene-1,5′-oxazolidin]-3′-yl)-N-(4-fluorobenzyl)acetamido)cyclobutyl)carbamate

The title compound was prepared as described in EXAMPLE 306, substituting (R)-2-(5-bromo-2′,4′-dioxo-2,3-dihydrospiro[indene-1,5′-oxazolidin]-3′-yl)acetic acid for cyclopropanecarboxylic acid and tert-butyl (3-((4-fluorobenzyl)amino)cyclobutyl)carbamate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 1016B

### (R)-tert-butyl (3-(N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamido)cyclobutyl)carbamate

The title compound was prepared as described in EXAMPLE 295, substituting (R)-tert-butyl (3-(2-(5-bromo-2′,4′-dioxo-2,3-dihydrospiro[indene-1,5′-oxazolidin]-3′-yl)-N-(4-fluorobenzyl)acetamido)cyclobutyl)carbamate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol.

### Example 1016C

### (R)—N-(3-aminocyclobutyl)-N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamide

The title compound was prepared as described in EXAMPLE 302, substituting (R)-tert-butyl (3-(N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamido)cyclobutyl)carbamate for tert-butyl 3-[(1R)-3′-(2-{benzyl[(1S)-1-cyclopropylethyl]amino}-2-oxoethyl)-2′,4′-dioxo-2,3-dihydrospiro[indene-1,5-[1,3]oxazolidin]-5-yl]azetidine-1-carboxylate.

### Example 1016D

### N-(4-fluorobenzyl)-2-[(1R)-5-(1-methyl-1H-pyrazol-4-yl)-2′,4′-dioxo-2,3-dihydro-3′H-spiro[indene-1,5′-[1,3]oxazolidin]-3′-yl]-N-(3-{[(trifluoromethyl)sulfonyl]amino}cyclobutyl)acetamide

The title compound was prepared as described in EXAMPLE 308, substituting (R)—N-(3-aminocyclobutyl)-N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamide for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide hydrochloride and trifluoromethanesulfonyl chloride for methanesulfonyl chloride. ¹H NMR (400 MHz, DMSO-d₆) δ 10.00-9.58 (m, 1H), 8.14 (d, J=6.9 Hz, 1H), 7.87 (d, J=6.3 Hz, 1H), 7.62-7.35 (m, 3H), 7.31-7.06 (m, 4H), 5.13-4.48 (m, 3H), 4.31 (d, J=17.9 Hz, 1H), 3.95-3.83 (m, 5H), 3.27-2.92 (m, 2H), 2.74-2.40 (m, 3H), 2.37-2.00 (m, 2H). LC-MS (APCI): 650 (M+H)⁺.

### Example 1017

### N-(4-fluorobenzyl)-2-[(1R)-5-(1-methyl-1H-pyrazol-4-yl)-2′,4′-dioxo-2,3-dihydro-3′H-spiro[indene-1,5′-[1,3]oxazolidin]-3′-yl]-N-[1-(methylsulfonyl)piperidin-4-yl]acetamide

### Example 1017A

### (R)-tert-butyl 4-(2-(5-bromo-2′,4′-dioxo-2,3-dihydrospiro[indene-1,5′-oxazolidin]-3′-yl)-N-(4-fluorobenzyl)acetamido)piperidine-1-carboxylate

The title compound was prepared as described in EXAMPLE 306, substituting (R)-2-(5-bromo-2′,4′-dioxo-2,3-dihydrospiro[indene-1,5′-oxazolidin]-3′-yl)acetic acid for cyclopropanecarboxylic acid and tert-butyl 4-((4-fluorobenzyl)amino)piperidine-1-carboxylate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 1017B

### (R)-tert-butyl 4-(N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamido)piperidine-1-carboxylate

The title compound was prepared as described in EXAMPLE 295, substituting (R)-tert-butyl 4-(2-(5-bromo-2′,4′-dioxo-2,3-dihydrospiro[indene-1,5′-oxazolidin]-3′-yl)-N-(4-fluorobenzyl)acetamido)piperidine-1-carboxylate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol.

### Example 1017C

### (R)—N-(4-fluorobenzyl)-2-(5-(1-methyl-1H-pyrazol-4-yl)-2′,4′-dioxo-2,3-dihydrospiro[indenene-1,5′-oxazolidin]-3′-yl)-N-(piperidin-4-yl)acetamide

The title compound was prepared as described in EXAMPLE 302, substituting (R)-tert-butyl 4-(N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamido)piperidine-1-carboxylate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 1017D

### N-(4-fluorobenzyl)-2-[(1R)-5-(1-methyl-1H-pyrazol-4-yl)-2′,4′-dioxo-2,3-dihydro-3′H-spiro[indene-1,5′-[1,3]oxazolidin]-3′-yl]-N-[1-(methylsulfonyl)piperidin-4-yl]acetamide

The title compound was prepared as described in EXAMPLE 308, substituting (R)—N-(4-fluorobenzyl)-2-(5-(1-methyl-1H-pyrazol-4-yl)-2′,4′-dioxo-2,3-dihydrospiro[indenene-1,5′-oxazolidin]-3′-yl)-N-(piperidin-4-yl)acetamide for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide hydrochloride. ¹H NMR (400 MHz, DMSO-d₆) δ 8.14 (d, J=7.6 Hz, 1H), 7.87 (dd, J=8.2, 0.8 Hz, 1H), 7.60-7.31 (m, 5H), 7.28-7.16 (m, 2H), 7.09 (t, J=8.9 Hz, 1H), 4.83-4.23 (m, 4H), 3.83 (d, J=3.9 Hz, 3H), 3.64-3.47 (m, 2H), 3.23-2.99 (m, 2H), 2.90-2.48 (m, 6H), 1.78-1.54 (m, 4H). LC-MS (APCI): 610 (M+H)⁺.

### Example 1018

### 2-[(1R,3S)-5-bromo-3-hydroxy-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

### Example 1018A

### (1R,3S)-5-bromo-3-hydroxy-2,3-dihydrospiro[indene-1,5′-oxazolidine]-2′,4′-dione

The title compound was prepared as described in EXAMPLE 698, substituting (R)-5-bromospiro[indene-1,5′-oxazolidine]-2′,3,4′(2H)-trione for N-(4-fluorobenzyl)-N—((S)-1,1,1,1-trifluoropropan-2-yl)-2-((S)-2,3′,5-trioxo-2′,3′-dihydrospiro[imidazolidine-4,1′-inden]-1-yl)acetamide.

### Example 1018B

### 2-[(1R,3S)-5-bromo-3-hydroxy-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 281F, substituting (1R,3S)-5-bromo-3-hydroxy-2,3-dihydrospiro[indene-1,5′-oxazolidine]-2′,4′-dione for (R)-5-bromo-2,3-dihydrospiro[indene-1,5′-oxazolidine]-2′,4′-dione. ¹H NMR (400 MHz, DMSO-d₆) δ 7.66 (d, J=1.8 Hz, 1H), 7.57 (dd, J=8.2, 1.9 Hz, 1H), 7.43-7.22 (m, 3H), 7.19-7.04 (m, 2H), 5.53 (s, 1H), 5.17 (p, J=7.5, 6.7 Hz, 2H), 4.88-4.19 (m, 4H), 3.02 (dd, J=14.2, 7.0 Hz, 1H), 2.30 (dd, J=14.3, 5.5 Hz, 1H), 1.35 (d, J=7.1 Hz, 3H). MS (ESI⁺) m/z 561 (M+H)⁺.

### Example 1019

### N-(4-fluorobenzyl)-2-[(1R,3S)-3-hydroxy-5-(1-methyl-1H-pyrazol-4-yl)-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-[(1R,3S)-5-bromo-3-hydroxy-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol. ¹H NMR (400 MHz, DMSO-d₆) δ 8.01 (s, 1H), 7.77 (s, 1H), 7.69-7.59 (m, 1H), 7.55 (dd, J=8.0, 1.6 Hz, 1H), 7.41-7.24 (m, 3H), 7.12 (t, J=8.7 Hz, 2H), 5.28-5.03 (m, 2H), 4.93-4.19 (m, 4H), 3.85 (d, J=1.0 Hz, 2H), 3.02 (dd, J=14.2, 6.9 Hz, 1H), 2.37-2.24 (m, 1H), 1.36 (d, J=7.1 Hz, 3H). MS (ESI⁺) m/z 561 (M+H)⁺.

### Example 1020

### 2,2′-({3-[(4-fluorobenzyl){[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]cyclobutyl}imino)diacetic acid

### Example 1020A

### (R)-tert-butyl (3-(2-(5-bromo-2′,4′-dioxo-2,3-dihydrospiro[indene-1,5′-oxazolidin]-3′-yl)-N-(4-fluorobenzyl)acetamido)cyclobutyl)carbamate

The title compound was prepared as described in EXAMPLE 306, substituting (R)-2-(5-bromo-2′,4′-dioxo-2,3-dihydrospiro[indene-1,5′-oxazolidin]-3′-yl)acetic acid for cyclopropanecarboxylic acid and tert-butyl (3-((4-fluorobenzyl)amino)cyclobutyl)carbamate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 1020B

### (R)-tert-butyl (3-(N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamido)cyclobutyl)carbamate

The title compound was prepared as described in EXAMPLE 295, substituting (R)-tert-butyl (3-(2-(5-bromo-2′,4′-dioxo-2,3-dihydrospiro[indene-1,5′-oxazolidin]-3′-yl)-N-(4-fluorobenzyl)acetamido)cyclobutyl)carbamate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol.

### Example 1020C

### (R)—N-(3-aminocyclobutyl)-N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamide

The title compound was prepared as described in EXAMPLE 302, substituting (R)-tert-butyl (3-(N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamido)cyclobutyl)carbamate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 1020D

### (R)-di-tert-butyl 2,2′-((3-(N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamido)cyclobutyl)azanediyl)diacetate

The title compound was prepared as described in EXAMPLE 832A, substituting (R)—N-(3-aminocyclobutyl)-N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamide for 5′-bromo-2′,3′-dihydrospiro[imidazolidine-4,1′-indene]-2,5-dione.

### Example 1020E

### 2,2′-({3-[(4-fluorobenzyl) {[(1R)-5-(1-methyl-1H-pyrazol-4-yl)-2′,4′-dioxo-2,3-dihydro-3′H-spiro[indene-1,5′-[1,3]oxazolidin]-3′-yl]acetyl}amino]cyclobutyl}imino)diacetic acid

The title compound was prepared as described in EXAMPLE 302, substituting (R)-di-tert-butyl 2,2′-((3-(N-(4-fluorobenzyl)-2-(5-(1-methyl-1H-pyrazol-4-yl)-2′,4′-dioxo-2,3-dihydrospiro[indene-1,5′-oxazolidin]-3′-yl)acetamido)cyclobutyl)azanediyl)diacetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 8.16 (d, J=6.5 Hz, 1H), 7.88 (d, J=7.1 Hz, 1H), 7.58 (s, 1H), 7.57-7.39 (m, 2H), 7.33-7.05 (m, 4H), 4.91-4.26 (m, 5H), 4.02-3.67 (m, 7H), 3.30-2.95 (m, 2H), 2.81-2.58 (m, 1H), 2.59-2.25 (m, 4H). LC-MS (APCI): 634 (M+H)⁺.

### Example 1021

### N-(4-fluorobenzyl)-2-[(1 S,3R)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-[(1S,3R)-5-bromo-3-fluoro-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆, 120° C.) δ ppm 8.15 (s, 1H), 7.89 (s, 1H), 7.85-7.72 (m, 2H), 7.59-7.42 (m, 2H), 7.39-7.29 (m, 2H), 7.17-7.08 (m, 2H), 6.20-6.01 (m, 1H), 5.26-5.09 (m, 1H), 4.87-4.78 (m, 1H), 4.76 (s, 2H), 4.72-4.55 (m, 2H), 4.45-4.31 (m, 1H), 3.26-3.08 (m, 1H), 2.74-2.59 (m, 4H), 1.37 (d, J=7.0 Hz, 3H). MS (ESI⁺) m/z 620 (M+H)⁺.

### Example 1022

### N-(4-fluorobenzyl)-2-[(1R)-5-(1,2-oxazol-4-yl)-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-[(1R)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 4-(4,4,5,5-tetramethyl-1,3,2-dioxaborolan-2-yl)isoxazole for 5-(4,4,5,5-tetramethyl-1,3,2-dioxaborolan-2-yl)pyridin-2-ol. ¹H NMR (400 MHz, DMSO-d₆, 120° C.) δ ppm 9.26 (d, J=1.7 Hz, 1H), 8.96 (d, J=1.6 Hz, 1H), 7.67 (s, 1H), 7.59 (d, J=8.0 Hz, 1H), 7.47 (d, J=8.0 Hz, 1H), 7.40-7.28 (m, 2H), 7.12 (t, J=8.8 Hz, 2H), 5.26-5.07 (m, 1H), 4.83 (d, J=17.6 Hz, 1H), 4.62 (d, J=17.9 Hz, 2H), 4.38 (d, J=16.8 Hz, 1H), 3.27-3.05 (m, 2H), 2.77-2.65 (m, 1H), 2.60-2.50 (m, 1H), 1.37 (d, J=7.0 Hz, 3H). MS (ESI⁺) m/z 532 (M+H)⁺.

### Example 1023

### N-(4-fluorobenzyl)-2-[(1S)-5-(1,2-oxazol-4-yl)-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-[(1S)-5-bromo-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 4-(4,4,5,5-tetramethyl-1,3,2-dioxaborolan-2-yl)isoxazole for 5-(4,4,5,5-tetramethyl-1,3,2-dioxaborolan-2-yl)pyridin-2-ol. ¹H NMR (400 MHz, DMSO-d₆, 120° C.) δ ppm 8.26 (brs, 1H), 7.92 (s, 1H), 7.54 (s, 1H), 7.48 (brs, 1H), 7.28-7.19 (m, 4H), 7.12-7.03 (m, 2H), 5.84 (brs, 1H), 5.33 (s, 2H), 4.83 (brs, 2H), 4.42-4.14 (m, 2H), 3.18-2.93 (m, 2H), 2.71-2.61 (m, 4H), 2.50-2.41 (m, 1H). MS (ESI⁺) m/z 532 (M+H)⁺.

### Example 1024

### N-(4-fluorobenzyl)-2-[(1R,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-[(1R,3S)-5-bromo-3-hydroxy-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.11 (s, 1H), 7.85 (s, 1H), 7.75-7.51 (m, 3H), 7.43-7.21 (m, 2H), 7.13 (t, J=8.6 Hz, 2H), 5.53 (dd, J=6.3, 2.1 Hz, 1H), 5.17 (q, J=6.0 Hz, 2H), 4.78 (d, J=20.5 Hz, 6H), 3.04 (s, 1H), 2.63 (d, J=4.7 Hz, 3H), 2.30 (dd, J=14.2, 5.2 Hz, 1H), 1.34 (d, J=7.0 Hz, 3H). MS (ESI⁺) m/z 618 (M+H)⁺.

### Example 1025

### N-(4-fluorobenzyl)-2-{(1 S,3R)-3-fluoro-5-[(methylcarbamoyl)amino]-2′,4′-dioxo-2,3-dihydro-3′H-spiro[indene-1,5′-[1,3]oxazolidin]-3′-yl}-N-[(2S)-1,1,1-trifluoropropan-2-yl]acetamide

### Example 1025A

### 2-((1S,3R)-5-amino-3-fluoro-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide

The title compound was prepared as described in EXAMPLE 284D-E, substituting 2-[(1 S,3R)-5-bromo-3-fluoro-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for N-benzyl-2-((S)-5-bromo-2′,4′-dioxo-2,3-dihydrospiro[indene-1,5′-oxazolidin]-3′-yl)-N—((S)-1-cyclopropylethyl)acetamide.

### Example 1025B

### N-(4-fluorobenzyl)-2-{(1 S,3R)-3-fluoro-5-[(methylcarbamoyl)amino]-2′,4′-dioxo-2,3-dihydro-3′H-spiro[indene-1,5′-[1,3]oxazolidin]-3′-yl}-N-[(2S)-1,1,1-trifluoropropan-2-yl]acetamide

The title compound was prepared as described in EXAMPLE 282, substituting 2-((1S,3R)-5-amino-3-fluoro-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for 2-[(1R)-5-amino-2′,4′-dioxo-2,3-dihydro-3′H-spiro[indene-1,5′-[1,3]oxazolidin]-3′-yl]-N-[(1R)-1-cyclopropylethyl]-N-(4-fluorobenzyl)acetamide and methanamine hydrochloride for 3,3-difluoroazetidine hydrochloride. ¹H NMR (400 MHz, DMSO-d₆, 120° C.) δ ppm 8.46 (brs, 1H), 7.75 (brs, 1H), 7.49-7.40 (m, 1H), 7.38-7.24 (m, 3H), 7.11 (t, J=8.7 Hz, 2H), 6.12-5.83 (m, 2H), 5.21-5.08 (m, 1H), 4.81 (d, J=17.6 Hz, 1H), 4.71-4.52 (m, 2H), 4.35 (d, J=16.8 Hz, 1H), 3.18-3.03 (m, 1H), 2.71-2.53 (m, 4H), 1.36 (d, J=7.1 Hz, 3H). MS (APCI⁺) m/z 555 (M+H)⁺.

### Example 1026

### (4-{[(5′-bromo-6′-fluoro-2,5-dioxo-2′,3′-dihydro-1H-spiro[imidazolidine-4,1′-inden]-1-yl)acetyl](4-fluorobenzyl)amino}cyclohexylidene)acetic acid

### Example 1026A

### tert-butyl 2-(5′-bromo-6′-fluoro-2,5-dioxo-2′,3′-dihydrospiro[imidazolidine-4,1′-inden]-1-yl)acetate

The title compound was prepared as described in EXAMPLE 832A, substituting 5′-bromo-6′-fluoro-2′,3′-dihydrospiro[imidazolidine-4,1′-indene]-2,5-dione for 5′-bromo-2′,3′-dihydrospiro[imidazolidine-4,1′-indene]-2,5-dione.

### Example 1026B

### 2-(5′-bromo-6′-fluoro-2,5-dioxo-2′,3′-dihydrospiro[imidazolidine-4,1′-inden]-1-yl)acetic acid

The title compound was prepared as described in EXAMPLE 302, substituting tert-butyl 2-(5′-bromo-6′-fluoro-2,5-dioxo-2′,3′-dihydrospiro[imidazolidine-4,1′-inden]-1-yl)acetate for tert-butyl 3-[(1R)-3′-(2-{benzyl[(1 S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 1026C

### tert-butyl 2-(4-(2-(5′-bromo-6′-fluoro-2,5-dioxo-2′,3′-dihydrospiro[imidazolidine-4,1′-inden]-1-yl)-N-(4-fluorobenzyl)acetamido)cyclohexylidene)acetate

The title compound was prepared as described in EXAMPLE 306, substituting 2-(5′-bromo-6′-fluoro-2,5-dioxo-2′,3′-dihydrospiro[imidazolidine-4,1′-inden]-1-yl)acetic acid for cyclopropanecarboxylic acid and tert-butyl 2-(4-((4-fluorobenzyl)amino)cyclohexylidene)acetate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 1026D

### (4-{[(5′-bromo-6′-fluoro-2,5-dioxo-2′,3′-dihydro-1H-spiro[imidazolidine-4,1′-inden]-1-yl)acetyl](4-fluorobenzyl)amino}cyclohexylidene)acetic acid

The title compound was prepared as described in EXAMPLE 302, substituting tert-butyl 2-(4-(2-(5′-bromo-6′-fluoro-2,5-dioxo-2′,3′-dihydrospiro[imidazolidine-4,1′-inden]-1-yl)-N-(4-fluorobenzyl)acetamido)cyclohexylidene)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (501 MHz, DMSO-d₆) δ 12.01 (s, 1H), 8.88 (d, J=15.5 Hz, 1H), 7.70 (d, J=6.6 Hz, 1H), 7.34 (dd, J=8.6, 5.2 Hz, 1H), 7.29-7.13 (m, 3H), 7.07 (t, J=8.9 Hz, 1H), 5.54 (d, J=10.3 Hz, 1H), 4.63-4.08 (m, 5H), 3.73 (dd, J=13.3, 8.6 Hz, 1H), 3.01 (q, J=7.8, 7.4 Hz, 2H), 2.65-2.52 (m, 1H), 2.25 (dtd, J=23.0, 17.7, 17.1, 9.5 Hz, 3H), 2.01-1.80 (m, 1H), 1.70 (dd, J=47.0, 11.6 Hz, 2H), 1.48 (dqd, J=39.5, 13.4, 13.0, 6.7 Hz, 2H). MS (ESI⁺) m/z 601.9 (M+H)⁺.

### Example 1027

### {4-[(4-fluorobenzyl){[6′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid

### Example 1027A

### tert-butyl 2-(4-(2-(6′-fluoro-5′-(1-methyl-1H-pyrazol-4-yl)-2,5-dioxo-2′,3′-dihydrospiro[imidazolidine-4,1′-inden]-1-yl)-N-(4-fluorobenzyl)acetamido)cyclohexylidene)acetate

The title compound was prepared as described in EXAMPLE 295, substituting tert-butyl 2-(4-(2-(5′-bromo-6′-fluoro-2,5-dioxo-2′,3′-dihydrospiro[imidazolidine-4,1′-inden]-1-yl)-N-(4-fluorobenzyl)acetamido)cyclohexylidene)acetate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol.

### Example 1027B

### {4-[(4-fluorobenzyl) {[6′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid

The title compound was prepared as described in EXAMPLE 302, substituting tert-butyl 2-(4-(2-(6′-fluoro-5′-(1-methyl-1H-pyrazol-4-yl)-2,5-dioxo-2′,3′-dihydrospiro[imidazolidine-4, 1′-inden]-1-yl)-N-(4-fluorobenzyl)acetamido)cyclohexylidene)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (500 MHz, DMSO-d₆) δ 11.99 (s, 1H), 8.84 (d, J=15.1 Hz, 1H), 8.11 (dd, J=7.7, 2.4 Hz, 1H), 7.88 (d, J=5.0 Hz, 1H), 7.65 (d, J=7.0 Hz, 1H), 7.36 (dd, J=8.5, 5.4 Hz, 1H), 7.23-7.04 (m, 4H), 5.55 (d, J=10.0 Hz, 1H), 4.63-4.09 (m, 5H), 3.87 (d, J=4.1 Hz, 3H), 3.78-3.69 (m, 1H), 3.02 (q, J=7.8 Hz, 2H), 2.64-2.52 (m, 1H), 2.36-2.19 (m, 3H), 1.97 (dt, J=15.6, 7.9 Hz, 1H), 1.80-1.61 (m, 2H), 1.61-1.35 (m, 2H). MS (ESI⁺) m/z 604.1 (M+H)⁺.

### Example 1028

### tert-butyl (4-{[(5′-bromo-6′-fluoro-2,5-dioxo-2′,3′-dihydro-1H-spiro[imidazolidine-4,1′-inden]-1-yl)acetyl](4-fluorobenzyl)amino}cyclohexylidene)acetate

The title compound was prepared as described in EXAMPLE 1026C. ¹H NMR (400 MHz, DMSO-d₆) δ 8.87 (d, J=12.4 Hz, 1H), 7.70 (d, J=6.4 Hz, 1H), 7.34 (dd, J=8.4, 5.4 Hz, 1H), 7.30-7.13 (m, 3H), 7.07 (t, J=8.7 Hz, 1H), 5.51 (d, J=7.9 Hz, 1H), 4.63-4.06 (m, 5H), 3.68 (d, J=12.1 Hz, 1H), 3.01 (q, J=7.1 Hz, 2H), 2.58 (dq, J=13.9, 7.2 Hz, 1H), 2.34-2.14 (m, 3H), 2.04-1.80 (m, 1H), 1.79-1.41 (m, 4H), 1.38 (d, J=3.2 Hz, 9H). MS (ESI⁻) m/z 658.2 (M−H)⁻.

### Example 1029

### 2-[(1R,3S)-5-bromo-3-fluoro-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide

The title compound was prepared as described in EXAMPLE 887, substituting 2-((1R,3R)-5-bromo-3-hydroxy-2′,4′-dioxo-2,3-dihydrospiro[indene-1,5′-oxazolidin]-3′-yl)-N-(4-fluorobenzyl)-N—((S)-1,1,1-trifluoropropan-2-yl)acetamide for N-(4-fluorobenzyl)-2-[(1 S,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide. ¹H NMR (400 MHz, DMSO-d₆) δ 7.95 (t, J=1.8 Hz, 1H), 7.91-7.70 (m, 1H), 7.53 (dd, J=14.9, 8.2 Hz, 1H), 7.36 (dd, J=8.5, 5.3 Hz, 1H), 7.22 (t, J=8.1 Hz, 2H), 7.12 (t, J=8.8 Hz, 1H), 6.13 (dt, J=55.6, 6.5 Hz, 1H), 5.38 (p, J=7.8 Hz, 1H), 5.11-4.09 (m, 4H), 3.11 (ddt, J=22.8, 15.7, 7.5 Hz, 1H), 2.86-2.56 (m, 1H), 1.35 (dd, J=15.2, 6.9 Hz, 3H). MS (ESI⁺) m/z 563 (M+H)⁺.

### Example 1030

### N-(4-fluorobenzyl)-2-[(1R,3S)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 295, substituting 2-[(1R,3S)-5-bromo-3-fluoro-2′,4′-dioxo-2,3-dihydro-3′H-spiro[indene-1,5′-[1,3]oxazolidin]-3′-yl]-N-(4-fluorobenzyl)-N-[(2S)-1,1,1-trifluoropropan-2-yl]acetamide for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and N-methyl-2-(4-(4,4,5,5-tetramethyl-1,3,2-dioxaborolan-2-yl)-1H-pyrazol-1-yl)acetamide for 5-(4,4,5,5-tetramethyl-1,3,2-dioxaborolan-2-yl)pyridin-2-ol. ¹H NMR (400 MHz, DMSO-d₆) δ 8.15 (s, 1H), 7.89 (s, 1H), 7.81-7.70 (m, 2H), 7.51 (dd, J=15.2, 7.8 Hz, 2H), 7.32 (ddd, J=9.5, 6.6, 3.3 Hz, 2H), 7.21-7.05 (m, 2H), 6.11 (ddd, J=56.2, 6.4, 2.4 Hz, 1H), 5.26-5.11 (m, 1H), 4.95-4.32 (m, 6H), 3.16 (ddd, J=20.7, 15.9, 6.4 Hz, 1H), 2.77-2.56 (m, 4H), 1.37 (d, J=7.0 Hz, 3H). MS (ESI⁺) m/z 620 (M+H)⁺.

### Example 1031

### {4-[(4-fluorobenzyl){[(1 S,3R)-3-fluoro-5-(1-methyl-1H-pyrazol-4-yl)-2′,4′-dioxo-2,3-dihydro-3′H-spiro[indene-1,5′-[1,3]oxazolidin]-3′-yl]acetyl}amino]cyclohexylidene}acetic acid

### Example 1031A

### tert-butyl 2-((1 S,3S)-5-bromo-3-hydroxy-2′,4′-dioxo-2,3-dihydrospiro[indene-1,5′-oxazolidin]-3′-yl)acetate

The title compound was prepared as described in EXAMPLE 832A, substituting (1S,3S)-5-bromo-3-hydroxy-2,3-dihydrospiro[indene-1,5′-oxazolidine]-2′,4′-dione for 5′-bromo-2′,3′-dihydrospiro[imidazolidine-4,1′-indene]-2,5-dione.

### Example 1031B

### tert-butyl 2-((1S,3R)-5-bromo-3-fluoro-2′,4′-dioxo-2,3-dihydrospiro[indene-1,5′-oxazolidin]-3′-yl)acetate

The title compound was prepared as described in EXAMPLE 887, substituting tert-butyl 2-((1S,3S)-5-bromo-3-hydroxy-2′,4′-dioxo-2,3-dihydrospiro[indene-1,5′-oxazolidin]-3′-yl)acetate for N-(4-fluorobenzyl)-2-[(1 S,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide.

### Example 1031C

### tert-butyl 2-((1 S,3R)-3-fluoro-5-(1-methyl-1H-pyrazol-4-yl)-2′,4′-dioxo-2,3-dihydrospiro[indene-1,5′-oxazolidin]-3′-yl)acetate

The title compound was prepared as described in EXAMPLE 295, substituting tert-butyl 2-((1S,3R)-5-bromo-3-fluoro-2′,4′-dioxo-2,3-dihydrospiro[indene-1,5′-oxazolidin]-3′-yl)acetate for 2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide and 1-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)pyridin-2-ol.

### Example 1031D

### 2-((1 S,3R)-3-fluoro-5-(1-methyl-1H-pyrazol-4-yl)-2′,4′-dioxo-2,3-dihydrospiro[indene-1,5′-oxazolidin]-3′-yl)acetic acid

The title compound was prepared as described in EXAMPLE 302, substituting tert-butyl 2-((1 S,3R)-3-fluoro-5-(1-methyl-1H-pyrazol-4-yl)-2′,4′-dioxo-2,3-dihydrospiro[indene-1,5′-oxazolidin]-3′-yl)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate.

### Example 1031E

### tert-butyl 2-(4-(2-((1S,3R)-3-fluoro-5-(1-methyl-1H-pyrazol-4-yl)-2′,4′-dioxo-2,3-dihydrospiro[indene-1,5′-oxazolidin]-3′-yl)-N-(4-fluorobenzyl)acetamido)cyclohexylidene)acetate

The title compound was prepared as described in EXAMPLE 306, substituting 2-((1S,3R)-3-fluoro-5-(1-methyl-1H-pyrazol-4-yl)-2′,4′-dioxo-2,3-dihydrospiro[indene-1,5′-oxazolidin]-3′-yl)acetic acid for cyclopropanecarboxylic acid and tert-butyl 2-(4-((4-fluorobenzyl)amino)cyclohexylidene)acetate for 2-[(1R)-5-(azetidin-3-yl)-2′,4′-dioxo-2,3-dihydro-3′H-spiro[indene-1,5′-[1,3]oxazolidin]-3′-yl]-N-benzyl-N-[(1S)-1-cyclopropylethyl]acetamide.

### Example 1031F

### {4-[(4-fluorobenzyl) {[(1 S,3R)-3-fluoro-5-(1-methyl-1H-pyrazol-4-yl)-2′,4′-dioxo-2,3-dihydro-3′H-spiro[indene-1,5′-[1,3]oxazolidin]-3′-yl]acetyl}amino]cyclohexylidene}acetic acid

The title compound was prepared as described in EXAMPLE 302, substituting tert-butyl 2-(4-(2-((1 S,3R)-3-fluoro-5-(1-methyl-1H-pyrazol-4-yl)-2′,4′-dioxo-2,3-dihydrospiro[indene-1,5′-oxazolidin]-3′-yl)-N-(4-fluorobenzyl)acetamido)cyclohexylidene)acetate for tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]oxazolidin]-5-yl]azetidine-1-carboxylate. ¹H NMR (400 MHz, DMSO-d₆) δ 8.08 (s, 1H), 7.88-7.65 (m, 3H), 7.55 (d, J=8.0 Hz, 1H), 7.30 (dd, J=8.3, 5.4 Hz, 2H), 7.08 (t, J=8.6 Hz, 2H), 6.10 (ddd, J=56.2, 6.4, 2.4 Hz, 1H), 4.55 (d, J=21.7 Hz, 6H), 3.86 (s, 3H), 3.74-3.65 (m, 1H), 2.66 (ddd, J=21.7, 15.7, 2.3 Hz, 2H), 2.40-2.22 (m, 2H), 2.06-0.68 (m, 9H). MS (ESI⁺) m/z 605 (M+H)⁺.

### Example 1032

### N-(4-fluorobenzyl)-2-[(1R,3R)-3-fluoro-5-(1-methyl-1H-pyrazol-4-yl)-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 887, substituting N-(4-fluorobenzyl)-2-[(1R,3S)-3-hydroxy-5-(1-methyl-1H-pyrazol-4-yl)-2′,4′-dioxo-2,3-dihydro-3′H-spiro[indene-1,5′-[1,3]oxazolidin]-3′-yl]-N-[(2S)-1,1,1-trifluoropropan-2-yl]acetamide for N-(4-fluorobenzyl)-2-[(1 S,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide. ¹H NMR (400 MHz, DMSO-d₆) δ 8.09 (s, 1H), 7.83 (s, 1H), 7.81-7.59 (m, 2H), 7.50 (m, 1H), 7.33 (t, J=7.0 Hz, 2H), 7.13 (t, J=8.8 Hz, 2H), 6.32 (ddd, J=56.2, 6.4, 2.4 Hz, 1H), 5.19 (s, 1H), 4.86-4.35 (m, 4H), 3.86 (s, 3H), 3.30 (t, J=5.6 Hz, 1H), 3.08 (s, 1H), 1.37 (d, J=7.1 Hz, 3H). MS (ESI⁺) m/z 563 (M+H)⁺.

### Example 1033

### N-(4-fluorobenzyl)-2-[(1R,3R)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide

The title compound was prepared as described in EXAMPLE 887, substituting N-(4-fluorobenzyl)-2-[(1R,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide for N-(4-fluorobenzyl)-2-[(1S,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]oxazolidin]-3′-yl]-N-[(2S)-1,1,1-trifluoropropan-2-yl]acetamide. ¹H NMR (400 MHz, DMSO-d₆) δ 8.15 (s, 1H), 7.89 (s, 1H), 7.81-7.62 (m, 2H), 7.51 (t, J=7.4 Hz, 2H), 7.33 (dd, J=8.4, 5.4 Hz, 2H), 7.13 (t, J=8.9 Hz, 2H), 6.32 (ddd, J=56.2, 6.4, 2.4 Hz, 1H),5.18 (s, 1H), 4.92-4.32 (m, 4H), 3.00-3.10 (m, 1H), 2.76-2.69 (m, 1H), 2.65 (d, J=4.7 Hz, 3H), 1.37 (d, J=7.1 Hz, 3H). MS (ESI⁺) m/z 620 (M+H)⁺.

### Testing of Representative Compounds

p300 HAT can acetylate all four core histones (H1A, H1B, H3, and H4), with acetylation occurring predominantly on the N-terminal amino acid residues. A p300 HAT assay in SPA format was designed using a substrate (Biotin-C6-GRGKGGKGLGKGGAK) comprising a synthetic peptide of 15 amino acids derived from the N-terminus of human Histone 4 (GRGKGGKGLGKGGAK (SEQ ID NO: 1)) that is chemically attached to biotin with an amino hexanoic acid linker (C6). The synthetic peptide was re-suspended in nanopure water; adjusted to pH 7.0 with concentrated NaOH; and subjected to amino acid analysis to estimate purity and concentration. Tritiated acetylCoA was diluted with cold acetylCoA (in water) as needed and used in the assay. A truncated variant of the HAT domain of the p300 enzyme was used in the assay. This variant consisted of residues 1287-1666 of p300 and was missing residues 1529-1560 of the autoacetylation loop. In addition, it contained the M1652G mutation as a result of the “expressed protein ligation” method that was used in its preparation, as previously described in the literature (Thompson et. al. 2004, Nature Structural & Molecular Biology, 11, 308-315). The test compounds were dissolved in DMSO to make 10 mM stocks and diluted further to make 4× stocks in 10 mM HEPES, pH 7.8 with 20% DMSO. The final concentration of DMSO in assays was kept at 5%. Compounds were tested at 11 concentrations ranging from 120 μM to 2 nM in 3-fold dilutions.

Assays were performed in a volume of 40 μL in a polypropylene 96 well plate. A typical reaction (40 μL) contained: 100 mM HEPES, pH 7.9, 100 mM KCl, 1 mM DTT, 50 μg/ml BSA, p300 HAT (˜5 nM), 0.01% Triton-X-100, and 5% DMSO. In addition, the substrate concentration was 12.5 μM biotinylated substrate peptide and 0.6 μM acetyl CoA.

Briefly, 20 μl of 2× reaction mixture (buffer ion, salt, substrate peptide, DTT, BSA, detergent, and enzyme) was incubated with 10 μl of 4× compound stock in 20% DMSO for 30 min. The reaction was initiated by the addition of 10 μl of 4× acetyl CoA and quenched with 120 μl of 0.5N HCl at required time intervals. The contents (˜160 μl) were delivered to the SPA plate coated with avidin and scintillant, incubated for 1 h, and light emission was counted in a TOP COUNT Microplate scintillation and luminescence counter (Perkin Elmer). IC₅₀ values were calculated based on percent inhibition calculated from these readings.

### Western Blot

To assess the efficacy of p300 inhibition in vitro, western blot analyses of histone, NF-κB, and p53 acetylation were performed in HeLa and HEK293 cell lines. p300/CBP dependent acetylation of lysines 18 and 27 of H3 histone (see Di Cerbo and Schneider., Briefings in Functional Genomics Advance Access, Jan. 15, 2013, pp 1-13), lysine 310 of the NF-κB p65 subunit, and lysine 382 of p53 (in HEK293 only, HeLa do not express p53) was monitored. The involvement of p300/CBP in acetylation at these residues was confirmed in preliminary experiments utilizing p300/CBP siRNA prior to experiments with inhibitors.

### Cell Treatment

HeLa and HEK 293 were maintained and passaged in EMEM (ATCC, Manassas, Va.) supplemented with 10% heat inactivated FBS, and were kept in a humidified incubator at 37° C. and 5% CO₂. For experiments, cells were seeded into BD BioCoat™ 6-well poly-D-lysine coated plates (3×10⁵ cells per well for HeLa and 6×10⁵ cells for HEK 293) in 2 ml complete media and incubated overnight at 37° C. and 5% CO₂.

For siRNA experiments, transfections were done the day after seeding. Cells were treated with a mixture of DharmaFECT formulation 1 and 25 pg/mL of p300 siRNA alone, both p300 and CBP siRNAs, or nontargeting siRNA (Thermo Scientific, Pittsburgh, Pa.) in complete media overnight. Treatments were aspirated off the following day and replaced with fresh media, and cells were incubated an additional 24 h. Cells were then treated with HDAC inhibitors and TNF-α as described below.

For inhibitor studies, compound dilutions were prepared in complete media from 10 mM stock solutions in DMSO. 2 mL of media containing the appropriate concentration of inhibitor was added to each well. The final DMSO concentration in all wells was kept at 0.3%. Compounds were allowed to incubate in the presence of compound for 1 h.

Cells were then treated with the HDAC inhibitors nicotinamide (3.3 mM) and trichostatin A (1.65 uM) for 1 h, followed by treatment with TNF-α (10 ng/ml) for 1 h prior to lysis. Upon harvest, the media was discarded, and cells were lysed in a lithium dodecyl sulfate buffer containing protease and phosphatase inhibitors and TurboDNase. Lysates were boiled for 5 minutes and then frozen for subsequent protein quantitation, electrophoresis, and blotting.

### Protein Measurement

Protein of the samples is measured using BCA (Bicinchoninic Acid) Protein assay kit (Thermo Scientific, product #23227). Protein level is normalized across samples and prepared with sample buffer for loading into gels for electrophoresis.

### Western Blot

Prepared samples were loaded on to SDS-polyacrylamide gels using Novex® NuPAGE® gels and transferred to nitrocellulose. After 30 min block with PBS containing Tween and 5% nonfat dry milk at room temperature with constant agitation, overnight incubation with primary antibody was done. Primary antibodies included: anti-pan acetyl-lysine, anti-acetyl NF-κB (Ac K310), anti-total NF-κB, anti-acetyl histone 3 (Ac K18), anti-acetyl histone 3 (Ac K27), anti-total Histone 3, and anti-β-actin as load control (Cell Signaling Technology). The anti-p300 was obtained from Abcam, and anti-CBP was obtained from Santa Cruz. Anti-p53 acetyl-Lysine 382 was obtained from Cell Signaling Technology, and anti-total p53 antibody was obtained from Santa Cruz. On the following day, membranes were washed 5× for 5 min each time with PBS-T milk and incubated with secondary antibody for 3 h and washed 5× for 5 min each time. Secondary antibodies linked to horseradish peroxidase, including anti-rabbit IgG and anti-mouse IgG, were obtained from Life Technologies. Detection was done by chemiluminescence and measured with FluorChem imager (Protein Simple).

### CRE Luciferase Gene Reporter Assay

CRE luciferase reporter system has tandem repeats of the CRE transcriptional response element and basic promoter elements to drive the transcription of a downstream reporter gene (luciferase). P300, a transcriptional coactivator, binds CRE and drives the transcription of the downstream genes. Cignal CRE luciferase reporter assay kit (Qiagen (CCS-002L)) was used to establish a cell based assay for p300 function. HEK293 cells were purchased from ATCC and cultured in EMEM with 10% HI FBS (ATCC® 30-2003). To transfect the reporter in HEK 293 cells, Attractene, a transfection reagent from Qiagen (301005) was used.

HEK-293 cells, were grown in EMEM with 10% FBS to 70% confluency. The cells were harvested and plated in a 96 well Poly D-Lys plate (BIOCOAT® Becton and Dickinson) with a cell density of 60000 cells/well in 100 μL of EMEM with 10% HI FBS. In parallel, the transfection cocktail (provided by the kit) was prepared in plain EMEM with no FBS. Positive control (GFP) and negative control from the kit were included. The plate was treated with 25-50 μL of transfection cocktail and incubated at 37° C. for 18-20 h. The next day, the plate was gently tapped over an absorbent pad to remove media. 100 μL aliquots of compounds at various concentrations (in EMEM with 10% HI FBS and 0.4% DMSO) were delivered to the appropriate wells and incubated for 1 h at 37° C. The control wells received media (EMEM with 10% HI FBS and 0.4% DMSO) with no compound. The plate was then treated with Forskolin (final conc: 2 M) in EMEM (with 0.4% DMSO and 10% HI FBS) and incubated for 3 h at 37° C. After 3 h, the plate was gently tapped over an absorbent pad, treated with 50 μL of passive lysis buffer (from Qiagen, Dual Luciferase Reporter Kit), and incubated at RT for 20 min. The plate was subjected to a freeze-thaw cycle, and a 20 μL aliquot was assayed for luciferase activity using DLR Kit with a GloMAX® luminometer. The luminescence from “unstimulated” control wells was subtracted from the rest, and the EC₅₀ was calculated.

### NF-κB Luciferase Gene Reporter Assay

The protocol is identical to CRE luciferase gene reporter assay except Cignal NF-κB reporter assay kit from Qiagen (CCS-013L) was used, and human TNF-α (final conc: 5 ng/mL in EMEM with 0.4% DMSO and 10% FBS) was used for induction.

TABLE 2
Activity Data*

| No. | IC₅₀ | No. | IC₅₀ | No. | IC₅₀ |
|---|---|---|---|---|---|
| 2-1 | +++ | 3-1 | ++ | 4-1 | ++ |
| 5-1 | ++ | 6-1 | ++ | 7-1 | ++ |
| 8-1 | + | 9-1 | ++ | 10-1 | + |
| 11-1 | +++ | 12-1 | ++ | 13-1 | ++ |
| 14-1 | +++ | 15-1 | ++ | 16-1 | ++ |
| 17-1 | +++ | 18-1 | + | 19-1 | + |
| 20-1 | ++ | 21-1 | ++ | 22-1 | + |
| 23-1 | ++ | 24-1 | + | 25-1 | ++ |
| 26-1 | ++ | 27-1 | + | 28-1 | ++ |
| 29-1 | ++ | 30-1 | +++ | 31-1 | ++ |
| 32-1 | +++ | 33-1 | ++ | 34-1 | + |
| 35-1 | + | 36-1 | + | 37-1 | ++ |
| 38-1 | +++ | 39-1 | ++ | 40-1 | ++ |
| 41-1 | + | 42-1 | + | 43-1 | + |
| 44-1 | + | 45-1 | + | 46-1 | ++ |
| 47-1 | ++ | 48-1 | + | 49-1 | + |
| 50-1 | ++ | 51-1 | ++ | 52-1 | ++ |
| 53-1 | ++ | 54-1 | +++ | 55-1 | ++ |
| 56-1 | + | 57-1 | ++ | 58-1 | +++ |
| 59-1 | + | 60-1 | + | 61-1 | ++ |
| 62-1 | ++ | 63-1 | +++ | 64-1 | ++ |
| 65-1 | +++ | 66-1 | ++ | 67-1 | +++ |
| 68-1 | ++ | 69-1 | + | 70-1 | +++ |
| 71-1 | +++ | 72-1 | ++ | 73-1 | +++ |
| 74-1 | +++ | 75-1 | +++ | 76-1 | ++ |
| 77-1 | ++ | 77-2 | ++ | 78-1 | ++ |
| 79-1 | + | 80-1 | + | 81-1 | + |
| 82-1 | + | 83-1 | ++ | 84-1 | ++ |
| 85-1 | + | 86-1 | + | 87-1 | + |
| 88-1 | +++ | 89-1 | ++ | 90-1 | +++ |
| 91-1 | +++ | 92-1 | +++ | 93-1 | ++ |
| 94-1 | +++ | 95-1 | + | 96-1 | +++ |
| 97-1 | +++ | 98-1 | +++ | 99-1 | ++ |
| 100-1 | ++ | 101-1 | ++ | 102-1 | + |
| 103-1 | + | 104-1 | + | 105-1 | +++ |
| 106-1 | +++ | 107-1 | + | 108-1 | +++ |
| 109-1 | +++ | 110-1 | + | 111-1 | + |
| 112-1 | +++ | 113-1 | + | 114-1 | +++ |
| 115-1 | + | 116-1 | +++ | 117-1 | +++ |
| 118-1 | +++ | 119-1 | +++ | 120-1 | + |
| 121-1 | +++ | 122-1 | ++ | 123-1 | +++ |
| 124-1 | +++ | 125-1 | ++ | 126-1 | +++ |
| 127-1 | ++ | 128-1 | + | 129-1 | +++ |
| 130-1 | ++ | 131-1 | + | 132-1 | + |
| 133-1 | + | 134-1 | +++ | 135-1 | +++ |
| 136-1 | ++ | 137-1 | +++ | 138-1 | ++ |
| 139-1 | + | 140-1 | ++ | 141-1 | +++ |
| 142-1 | ++ | 143-1 | ++ | 144-1 | +++ |
| 145-1 | + | 146-1 | ++ | 147-1 | +++ |
| 148-1 | +++ | 149-1 | + | 150-1 | ++ |
| 151-1 | ++ | 152-1 | +++ | 153-1 | + |
| 154-1 | + | 155-1 | +++ | 156-1 | ++ |
| 157-1 | +++ | 158-1 | +++ | 159-1 | ++ |
| 160-1 | ++ | 161-1 | +++ | 162-1 | +++ |
| 163-1 | +++ | 164-1 | +++ | 165-1 | +++ |
| 166-1 | ++ | 167-1 | +++ | 168-1 | +++ |
| 169-1 | +++ | 170-1 | +++ | 171-1 | +++ |
| 172-1 | +++ | 173-1 | + | 174-1 | +++ |
| 175-1 | +++ | 176-1 | +++ | 177-1 | +++ |
| 178-1 | +++ | 179-1 | +++ | 180-1 | +++ |
| 181-1 | +++ | 182-1 | +++ | 183-1 | +++ |
| 184-1 | +++ | 185-1 | +++ | 186-1 | +++ |
| 187-1 | + | 188-1 | +++ | 189-1 | +++ |
| 190-1 | ++ | 191-1 | ++ | 192-1 | + |
| 193-1 | +++ | 194-1 | +++ | 194-2 | +++ |
| 195-1 | +++ | 196-1 | +++ | 197-1 | +++ |
| 198-1 | +++ | 199-1 | +++ | 200-1 | +++ |
| 201-1 | ++ | 202-1 | +++ | 203-1 | +++ |
| 204-1 | ++ | 205-1 | +++ | 206-1 | +++ |
| 207-1 | +++ | 208-1 | +++ | 209-1 | +++ |
| 210-1 | ++ | 211-1 | +++ | 212-1 | +++ |
| 213-1 | +++ | 214-1 | +++ | 215-1 | +++ |
| 216-1 | +++ | 217-1 | +++ | 218-1 | +++ |
| 219-1 | +++ | 220-1 | ++ | 221-1 | ++ |
| 222-1 | +++ | 223-1 | +++ | 224-1 | ++ |
| 225-1 | +++ | 226-1 | +++ | 227-1 | +++ |
| 228-1 | +++ | 229-1 | +++ | 230-1 | +++ |
| 231-1 | +++ | 232-1 | +++ | 233-1 | +++ |
| 234-1 | +++ | 235-1 | +++ | 235-2 | +++ |
| 236-1 | +++ | 237-1 | +++ | 238-1 | +++ |
| 239-1 | +++ | 240-1 | +++ | 241-1 | +++ |
| 242-1 | + | 243-1 | + | 244-1 | + |
| 245-1 | +++ | 246-1 | +++ | 247-1 | ++ |
| 248-1 | +++ | 249-1 | +++ | 250-1 | + |
| 251-1 | ++ | 252-1 | +++ | 253-1 | +++ |
| 254-1 | ++ | 255-1 | + | 256-1 | +++ |
| 257-1 | + | 258-1 | + | 259-1 | +++ |
| 260-1 | +++ | 261-1 | +++ | 261-2 | +++ |
| 262-1 | +++ | 263-1 | +++ | 264-1 | +++ |
| 265-1 | +++ | 265-2 | +++ | 266-1 | +++ |
| 266-2 | +++ | 267-1 | +++ | 267-2 | +++ |
| 268-1 | +++ | 268-2 | +++ | 268-3 | +++ |
| 268-4 | +++ | 269-1 | +++ | 269-2 | +++ |
| 270-1 | +++ | 270-2 | +++ | 271-1 | +++ |
| 271-2 | +++ | 272-1 | +++ | 272-2 | +++ |
| 273-1 | +++ | 273-2 | +++ | 274-1 | +++ |
| 275-1 | +++ | 275-2 | +++ | 276-1 | ++ |
| 277-1 | ++ | 278-1 | +++ | 279-1 | +++ |

+++ indicates IC₅₀ of 1 μM or less
++ indicates IC₅₀ between 1 μM and 10 μM
+ indicates IC₅₀ from 10 μM to 120 μM

### General ELISA Protocol

### Cell Lines and Culture Conditions

All cancer cell lines were maintained and passaged in 75 cm² poly-D-lysine coated tissue culture flasks in a humidified tissue culture incubator kept at 37° C. and 5% CO₂. DU 145 were cultured and assayed in EMEM supplemented with 10% heat inactivated FBS, PC-3 in RPMI-1640 with 10% heat inactivated FBS, Kasumi-1 in RPMI-1640+20% FBS, and SKNO-1 in RPMI-1640+10% FBS+10 ng/mL G-CSF. All cells were assayed in the media in which they were maintained. For experiments with human PBMCs, frozen vials were thawed and plated directly into 48 well assay plates in RPMI-1640+10% FBS for use the following day.

### TNF-α Mediated IL-6 Production in Prostate Cancer Cell Lines

DU 145 and PC-3 prostate cancer cells were seeded into 12-well poly-D-lysine coated plates at 1.0×10⁵ cells per well in 500 μL complete media and incubated overnight at 37° C. and 5% CO₂. The following day, 3-fold serial dilutions of the compounds to be tested (10, 3.33, 1.11, 0.37, 0.12, 0.041 mM) were prepared in neat DMSO from a 10 mM stock, and 1 μL of each corresponding dilution was added to the appropriate wells (20, 6.67, 2.22, 0.74, 0.25, 0.082 μM final concentrations). After a 1 h pretreatment with p300 inhibitor, cells were treated with 10 ng/mL TNF-α (5 μL of a 1 μg/mL stock) for 24 h.

After 24 h of TNF treatment, cell supernatants were collected in 0.5 mL microfuge tubes, centrifuged at 1000 rpm for 10 min, transferred to a fresh tube, and stored at −80° C. Cell monolayers were lysed in 1× lithium dodecyl sulfate buffer, and protein content was measured using the Pierce BCA Protein Assay Kit. The amount of IL-6 in the cell supernatants was quantified using the SABiosciences Human IL-6 Single Analyte ELISArray Kit (Cat. No. SEH00560A) according to the manufacturer's protocol.

### Cobalt Chloride Mediated VEGF Production in DU 145 Cells

DU 145 cells were seeded into 48-well poly-D-lysine coated plates at 37,500 cells per well in 250 μL EMEM+10% FBS and incubated overnight at 37° C. and 5% CO₂. On the following day, three-fold serial dilutions of the compounds to be tested (10, 3.33, 1.11, 0.37, 0.12, 0.041 mM) were prepared in neat DMSO from a 10 mM stock, and then 4 μL of each dilution was added to 28 μL of complete media (1.25, 0.417, 0.139, 0.046, 0.015, 0.005 mM). Finally, 4 μL of each dilution was added to the appropriate wells (20, 6.67, 2.22, 0.74, 0.25, 0.082 μM final concentrations). After a 1 h pretreatment with compound, cells were treated with 250 μM cobalt chloride (5 μL of a 12.5 mM stock in complete media) for 48 h.

After 48 h of CoCl₂ treatment, cell supernatants were collected in 0.5 mL microfuge tubes, centrifuged at 1000 rpm for 10 min, transferred to a fresh tube, and stored at −80° C. Cell monolayers were lysed in 1× lithium dodecyl sulfate buffer, and protein content was measured using the Pierce BCA Protein Assay Kit. VEGF in the cell supernatants was quantitated using the Thermo Fisher Human VEGF ELISA Kit (Cat. No. EH2VEGF2) according to the manufacturer's protocol.

### TNF-α Mediated IL-8 Production in AML1-ETO Positive AML Cell Lines

Kasumi-1 and SKNO-1 AML cells were seeded into 12-well poly-D-lysine coated plates at 1.0×10⁵ cells per well in 500 μL complete media and incubated overnight at 37° C. and 5% CO₂. The following day, 3-fold serial dilutions of the compounds to be tested (10, 3.33, 1.11, 0.37, 0.12, 0.041 mM) were prepared in neat DMSO from a 10 mM stock, and 1 μL of each corresponding dilution is added to the appropriate wells (20, 6.67, 2.22, 0.74, 0.25, 0.082 μM final concentrations). After a 1 h pretreatment with p300 inhibitor, cells were treated with 10 ng/mL TNF-α (5 μL of a 1 μg/mL stock) for 24 h.

After 24 h of TNF treatment, the entire contents of each well were collected in 2.0 mL microfuge tubes, centrifuged at 1000 rpm for 10 min, and 1 mL of supernatant from each was transferred to a fresh tube and stored at −80° C. The remaining supernatant is aspirated from each tube and cell pellets were lysed in 1× lithium dodecyl sulfate buffer. Protein content was measured using the Pierce BCA Protein Assay Kit. IL-8 in the cell supernatants was quantified using the SABiosciences Human IL-8 Single Analyte ELISArray Kit (Cat. No. SEH00568A) according to the manufacturer's protocol.

### α-CD3/α-CD28 Mediated IFN-γ Production in Human PBMCs

PBMCs from human donors were thawed and plated in 48-well plates in 250 uL RPMI-1640+10% FBS and incubated overnight at 37° C. and 5% CO₂. The following day, 3-fold serial dilutions of compounds to be tested (10, 3.33, 1.11, 0.37, 0.12, 0.041 mM) were prepared in neat DMSO from a 10 mM stock, and then 5 μL of each dilution was added to 45 μL of complete media (1.0, 0.33, 0.11, 0.037, 0.012, 0.004 mM). Finally, 5 μL of each dilution was added to the appropriate wells (20, 6.67, 2.22, 0.74, 0.25, 0.082 M final concentrations). After a 1 h pretreatment with compounds, cells were treated with 0.5 μg/mL α-CD3 (5 μL of a 25 μg/mL stock in complete media)+1.0 μg/mL α-CD28 (5 μL of a 50 μg/mL stock in complete media) for 48 h.

After 48 h of α-CD3/α-CD28 treatment, the entire contents of each well was collected in 2.0 mL microfuge tubes, centrifuged at 1000 rpm for 10 min, and 1 mL of supernatant from each was transferred to a fresh tube and stored at −80° C. The remaining supernatant was aspirated from each tube and cell pellets were lysed in 1× lithium dodecyl sulfate buffer. Protein content was measured using the Pierce BCA Protein Assay Kit. IFN-γ in the cell supernatants was quantified using the SABiosciences Human IFN-γ Single Analyte ELISArray Kit (Cat. No. SEH00380A) according to the manufacturer's protocol.

### Standard SABiosciences Single Analyte ELISArray Protocol

Briefly, 50 μL of cell culture supernatant or IL-6/IL-8/IFN-γ standard was added to each well along with 50 μL of assay buffer, and the plate was sealed and incubated at room temperature for 2 h. Wells were then washed 3 times with 350 μL wash buffer. 100 μL detection antibody solution was then added to each well, and the plate was sealed and incubated at room temperature for 1 h. Wells were again washed 3 times with 350 μL wash buffer. 100 μL of avidin-HRP was added to each well, and the plate was sealed and incubated at room temperature for 30 minutes in the dark. Wells were then washed 4 times with 350 μL wash buffer. 100 μL of development solution was added to each well, and the plate was sealed and incubated at room temperature for 15 minutes in the dark, followed by the addition of 100 μL of stop solution to each well. The absorbance in each well was then measured at 450 nM, and the absorbance at 570 nM was subtracted to correct for optical imperfections in the plate. All cytokine and growth factor values were normalized to cellular protein content.

### Thermo Fisher Human VEGF ELISA Protocol

Briefly, 50 μL of cell culture supernatant or VEGF standard were added to each well along with 50 μL of standard diluent, and the plate was sealed and incubated at room temperature for 2 h. Wells were then washed 3 times with 350 μL wash buffer. 100 μL of biotinylated antibody reagent was then added to each well, and the plate was sealed and incubated at room temperature for 1 h. Wells were again washed 3 times with 350 μL wash buffer. 100 μL of streptavidin-HRP is added to each well, and the plate was sealed and incubated at room temperature for 1 h. Wells were then washed 3 times with 350 μL wash buffer. 100 μL of TMB substrate solution was added to each well, and the plate was sealed and incubated at room temperature for 30 minutes in the dark, followed by the addition of 100 μL of stop solution to each well. The absorbance in each well was then measured at 450 nM, and the absorbance at 550 nM was subtracted to correct for optical imperfections in the plate. All VEGF concentrations were normalized to cellular protein content.

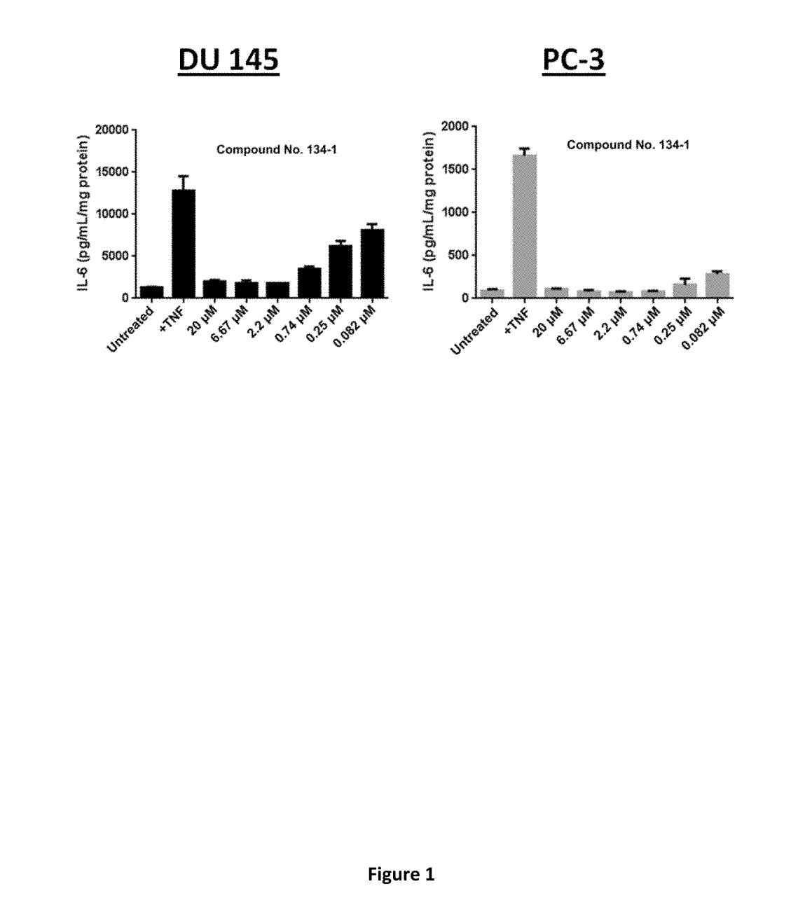

The effect of compound 134-1 on TNF-mediated IL-6 production is shown in FIG. 1. The calculated EC₅₀ value for 134-1 in DU145 and PC-3 cells is 145 nM and 19.7 nM, respectively.

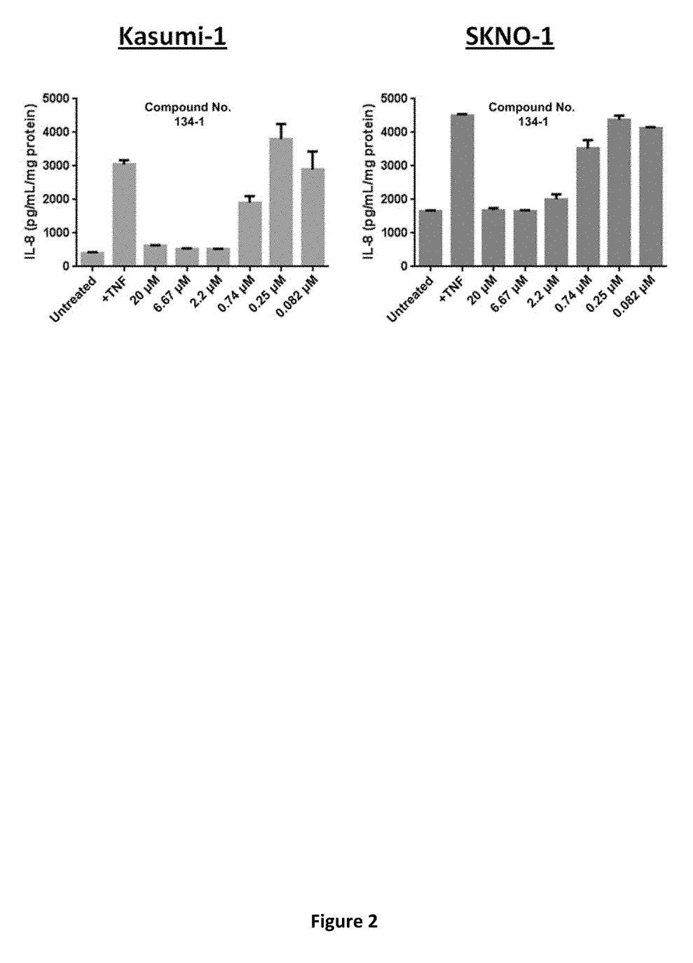

The effect of compound 134-1 on TNF-mediated IL-8 production is shown in FIG. 2. The calculated EC₅₀ value for 134-1 in Kasumi-1 and SKNO-1 cells is 755.8 nM and 974.5 nM, respectively.

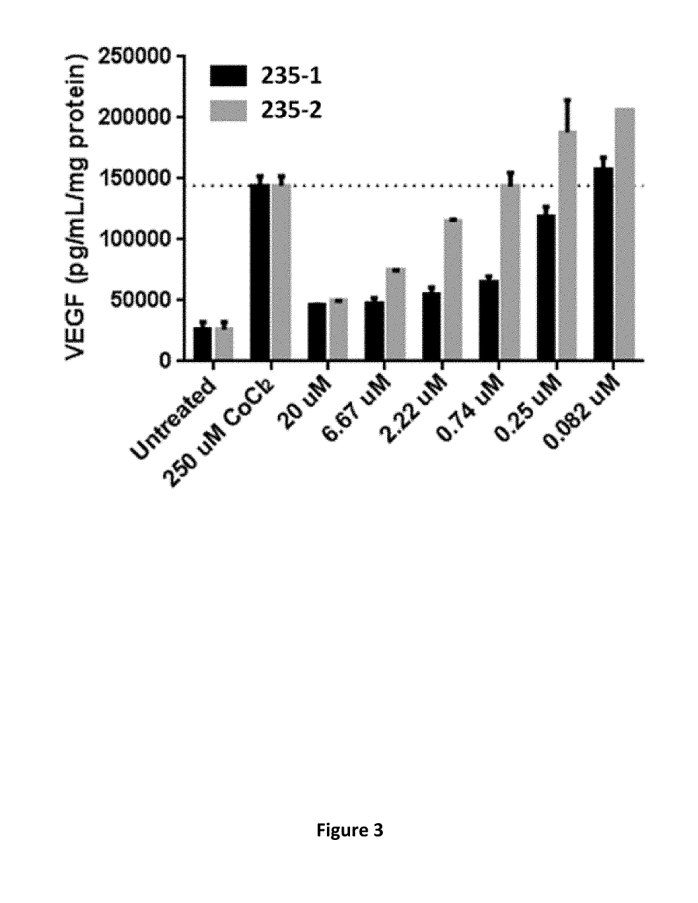

The effect of compounds 235-1 and 235-2 on CoCl2-mediated VEGF production is shown in FIG. 3. The calculated EC₅₀ value for 235-1 and 235-2 are 722 nM and 6,611 nM, respectively.

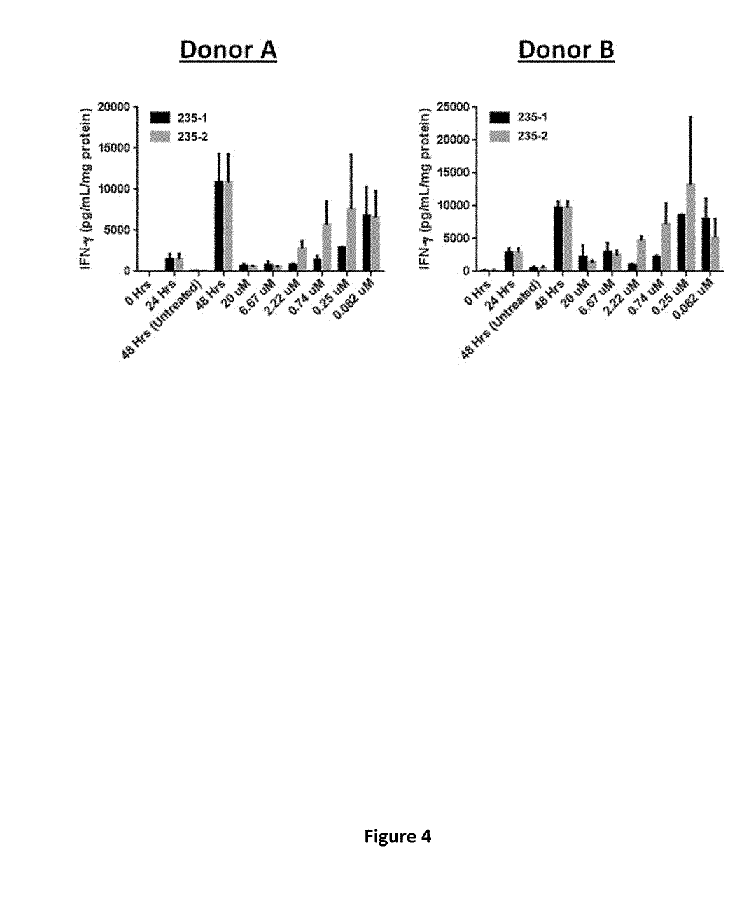

The effect of compounds 235-1 and 235-2 on IFN-γ production in PBMC derived from two different donors is shown in FIG. 4. The calculated EC₅₀ values for 235-1 and 235-2 in cells from donor A were 118.6 nM and 473.8 nM, respectively. The calculated EC₅₀ values for 235-1 and 235-2 in cells from donor B were 538.3 nM and 2,471 nM, respectively.

### Cell Proliferation Assay

### ³H-Thymidine Incorporation Assay to Measure Cell Proliferation

The protocol for the methyl-³H-thymidine incorporation assay to measure and rank the effect of our p300/CBP Histone Acetyl Transferase (HAT) inhibitors on the proliferation of a collection of cancer cell lines was adapted from Griffiths, Mand Sundaram, H. Drug Design and Testing: Profiling ofAntiproliferative Agents for Cancer Therapy Using a Cell-Based Methyl-[3H]-Thymidine Incorporation Assay. In: Cancer Cell Culture: Methods and Protocols, Second Edition, Methods in Molecular Biology, vol. 731, 451-465, 2011. Ian A. Cree (ed.), Springer Science+Business Media, LLC.

### Determination of Optimal Cell Seeding Density

As a first step the optimal number of cells seeded per well for each cancer cell line has to be determined. For scientific and logistical reasons the optimal seeding density was determined for a 72 h assay. Most tumor cell lines have an approximate doubling time of 24-48 h, thus to be able to observe an effect on cell proliferation 72 h seems an appropriate time period because the cells will undergo one or more divisions. Cells were counted using an inverted microscope, hemocytometer and trypan blue. Solid tumor cell lines were first treated with trypsin to prepare a single-cell suspension. For hematopoietic tumor cell lines a suspension containing 4×10⁵ viable cells/mL and for solid tumors a suspension containing 2×10⁵ viable cells/mL were prepared.

Cell suspensions are added to wells in row A of a 96-well sterile polystyrene surface treated culture plate and serial two-fold dilutions are performed across the plate. The plates are incubated at a temperature of 37° C. and an atmosphere of 5% CO₂ in air for 16-20 h after which 100 μL of fresh culture medium was added to each well. The plates were then returned to the incubator for an additional 72 h. During the last 6 h the cells are incubated in the presence of 0.1 mCi/well.

After 6 h the plates were harvested using a PerkinElmerFilterMate cell harvester and the contents of the wells collected on 96-well Unifilter plates. After drying the plates at 37° C. for greater than 16 h the plates, scintillation fluid was added to the wells and the radioactivity in each well determined using a TopCount NXT scintillation counter (PerkinElmer).

The log cell density was plotted vs. the calculated average cpm to determine the optimal cell density (exponential growth phase/rising part of the curve). The results are shown in the Table 3 below.

TABLE 3
Cell lines used and their optimal seeding density (72 h assay)

| Cell line | Type | Seeding Density (cells/well) |
|---|---|---|
| Kasumi-1 | AML (human) | 16000 |
| SKNO-1 | AML (human) | 16000 |
| HL-60 | AML (human) | 16000 |
| KG-1 | AML (human) | 16000 |
| RPMI 8226 | MM (human) | 8000 |
| SU-DHL8 | MM (human) | 5000 |
| EJM | MM (human) | 16000 |
| LP-1 | MM (human) | 40000 |
| K562 | CML (human) | 1250 |
| C1498 | AML (mouse) | 1367 |
| B16/F10 | Melanoma (mouse) | 625 |
| 4T1 | Breast carcinoma | 312 |
| EMT6 | Breast carcinoma | 625 |

### Determination Effect of HAT Inhibitors and Chemotherapeutic Agents on Cell Proliferation

Cells at their predetermined optimal cell density (see Table 3) were added to wells of 96-well sterile polystyrene surface treated culture plates in a volume of 150 L/well. The plates were incubated for 16-20 h in an incubator set to maintain a temperature of 37° C. and an atmosphere of 5% CO₂ in air.

The next day serial dilutions of HAT inhibitors and selected chemotherapeutics (cytarabine, doxorubicin and paclitaxel) were prepared as follows. HAT inhibitors were diluted to 5 mM in DMSO from a 10 mM stock solution. The 5 mM solution was further diluted 166.7-fold in the appropriate culture medium such that each cell line receives a 30 mM solution in culture medium and 0.6% DMSO. This solution was added to wells in row A of a 96-well plate and serial three-fold dilutions were performed across the plate.

Chemotherapeutics dilutions were made in a similar fashion, except that the mM solution is diluted 125-fold in the appropriate culture medium for each cell line to get a 40 mM solution in culture medium+0.8% DMSO. The 40 mM solution was then diluted 2.5-fold in the appropriate culture medium supplemented with 0.6% DMSO to get a 16 mM solution in 0.68% DMSO. This solution was added to wells in row A of a 96-well plate and serial four-fold dilutions were performed across the plate.

HAT inhibitor and chemotherapeutic agent dilutions were then added to corresponding wells of a 96-well plate containing cells seeded the day before, resulting in another 4-fold dilution and a final DMSO concentration in the wells of 0.2%. The final concentrations of the HAT inhibitors and chemotherapeutic agents in the assay are shown in Table 4.

TABLE 4
HAT inhibitor or chemotherapeutic agent concentrations in ³H-Thymidine
assay.

Concentration (mM)

| 96-well plate row | HAT inhibitor | Chemotherapeutic Agent |
|---|---|---|
| A | 7.5 | 4 |
| B | 2.5 | 0.8000 |
| C | 0.8333 | 0.1600 |
| D | 0.2778 | 0.0320 |
| E | 0.0926 | 0.0064 |
| F | 0.0309 | 0.0013 |
| G | 0.0103 | 0.0003 |
| H | 0.0034 | 0.0001 |

The plates were then incubated at 37° C., 5% CO₂ for an additional 72 h. During the last 6 h the cells are incubated in the presence of 0.1 mCi/well (20 mL of a 1:200 dilution of a 1 mCi/mL stock solution) and processed as described above for determination of the optimal cell density.

The mean cpm for each triplicate condition and the percent inhibition were calculated for each HAT inhibitor and chemotherapeutic concentration as follows: mean cpm in treatment group—mean cpm medium control (no cells)/mean cpm cells only-medium cpm medium control×100% (normalized response). The data were entered in GraphPad Prism and the IC₅₀ value for each inhibitor was determined using the log inhibitor concentration vs. normalized response-variable slope function.

### Synergy Determinations

Determination of the Effect Between HAT Inhibitors and Chemotherapeutic Agents and/or Bromodomain Inhibitors

To determine the effect of the combination of a HAT inhibitor with a chemotherapeutic agent and/or a bromodomain inhibitor on the proliferation of cancer cell lines the following approach was taken based on the method described by Chou and Talalay (Chou T C and Talalay P. Trends Pharmacol Sci 4; 450-454, 1983, Adv Enzyme Regul 22: 27-55, 1984, New Adventures in Developmental Cancer Chemotherapy. Harrap K R and Connors T A eds. P 37-64 Academic press, New York, 1987).

First the IC₅₀ of the individual compounds is determined as described in the ³H-Thymidine incorporation assay protocol, then the ratio of the IC₅₀s is calculated. The final concentrations of the HAT inhibitors, chemotherapeutic agents and/or bromodomain inhibitors in the assay are shown in Table 5.

TABLE 5
HAT inhibitor, chemotherapeutic agent and bromodomain inhibitor
concentrations either alone or in combination in the cell
proliferation assay

| | Fold IC₅₀ for each compound | | |
|---|---|---|---|
| | Individual compound | 2 compounds | 3 compounds |
| A | 8x | 8x | 8x |
| B | 4x | 4x | 4x |
| C | 2x | 2x | 2x |
| D | 0.5x | 0.5x | 0.5x |
| E | 0.25x | 0.25x | 0.25x |
| F | 0.125x | 0.125x | 0.125x |
| G | 0.0625x | 0.0625x | 0.0625x |
| H | 0.0313x | 0.0313x | 0.0313x |

The mean cpm for each triplicate condition and the percent inhibition was calculated for each individual compound concentration and each compound combination concentration as follows: mean cpm in treatment group—mean cpm medium control (no cells)/mean cpm cells only-medium cpm medium control×100% (normalized response). The data were entered in GraphPad Prism and the EC₉₀, EC₈₀, EC₇₀, EC₆₀, EC₅₀, EC₄₀, EC₃₀, EC₂₀ and EC₁₀ values for each individual compound and the combination(s) were calculated using the log (agonist) concentration vs. normalized response-find ECanything function.

The data were then entered into CompuSyn (T-C Thou and NMartin, version 1.0, ComboSyn, Inc. 2005) and the combination index (CI) can be calculated for each effect level. The average CI values at EC₅₀, EC₇₅, EC₉₀ and EC₉₅ were also considered as a measure of synergy/antagonism/additivity. See Table 6 for CI values and the associate effect of compound combinations.

TABLE 6
Combination Index value and the associated biological effect

| Range of Combination Index values | Effect |
|---|---|
| <0.1 | very strong synergism |
| 0.1-0.3 | strong synergism |
| 0.3-0.7 | synergism |
| 0.7-0.85 | moderate synergism |
| 0.85-0.90 | slight synergism |
| 0.90-1.10 | nearly additive |
| 1.10-1.20 | slight antagonism |
| 1.20-1.45 | moderate antagonism |
| 1.45-3.3 | antagonism |
| 3.3-10 | strong antagonism |
| >10 | very strong antagonism |

TABLE 7
HAT inhibitor 268-1 in combination with chemotherapeutic agents and/or
HDAC inhibitors and/or Bromodomain inhibitors on SKNO-1 acute
myeloid leukemia cells

| Combination 268-1 and | | |
|---|---|---|
| indicated chemotherapeutic | | |
| agent(s) | Combination Index | Effect |
| Cytarabine (AraC) | 1.19 | slight antagonism |
| Doxorubicin (Dox) | 1.32 | moderate antagonism |
| Paclitaxel (TXL) | 1.12 | slight antagonism |
| Gemcitabine (Gem) | 1.09 | nearly additive |
| Vinblastine (Vin) | 1.42 | moderate antagonism |
| Mitoxantrone (Mito) | 1.31 | moderate antagonism |
| Etoposide (VP16) | 1.34 | moderate antagonism |
| Trichostatin A (TSA) | 1.19 | slight antagonism |
| Apicidin (Api) | 1.39 | moderate antagonism |
| JQ-1 | 0.75 | moderate synergism |
| I-BET 762 | 0.77 | moderate synergism |
| TSA and JQ-1 | 1.11 | slight antagonism |
| TSA and I-BET 762 | 1.07 | nearly additive |
| Api and JQ-1 | 1.15 | slight antagonism |
| Api and I-BET 762 | 1.01 | nearly additive |
| AraC and JQ-1 | 1.52 | antagonism |
| AraC and I-BET 762 | 1.54 | antagonism |
| Dox and JQ-1 | 1.04 | nearly additive |
| Dox and I-BET 762 | 0.93 | nearly additive |

TABLE 8
HAT inhibitor 266-1 in combination with chemotherapeutic agents and/
or HDAC inhibitors and/or Bromodomain inhibitors on RPMI 8226
multiple myeloma cells

| Combination 266-1 and | | |
|---|---|---|
| indicated chemotherapeutic | | |
| agent(s) | Combination Index | Effect |
| Doxorubicin (Dox) | 1.27 | moderate antagonism |
| Gemcitabine (Gem) | 2.00 | antagonism |
| Etoposide (VP16) | 1.25 | moderate antagonism |
| Trichostatin A (TSA) | 1.10 | nearly additive |
| Apicidin (Api) | 2.74 | antagonism |
| JQ-1 | 0.68 | synergism |
| I-BET 762 | 0.48 | synergism |
| TSA and JQ-1 | 1.10 | nearly additive |
| TSA and I-BET 762 | 1.05 | nearly additive |
| Api and JQ-1 | 1.59 | antagonism |
| Api and I-BET 762 | 0.82 | moderate synergism |
| Dox and TSA | 1.20 | slight antagonism |
| Dox and Api | 6.13 | strong antagonism |
| Dox and JQ-1 | 1.13 | slight antagonism |
| Dox and I-BET 762 | 0.56 | synergism |
| Gem and TSA | 1.39 | moderate antagonism |
| Gem and Api | 1.81 | antagonism |
| Gem and JQ-1 | 1.44 | moderate antagonism |
| Gem and I-BET 762 | 1.09 | nearly additive |
| VP-16 and TSA | 1.37 | moderate antagonism |
| VP-16 and Api | 2.28 | antagonism |
| VP-16 and JQ-1 | 0.75 | moderate synergism |
| VP-16 and I-BET 762 | 0.51 | synergism |

TABLE 9
HAT inhibitors 235-1, 265-1 and 268-1 in combination with
chemotherapeutic agents and/or Bromodomain inhibitors on
DU-145 prostate cancer cells

| Combination of HAT inhibitor | | |
|---|---|---|
| and indicated chemotherapeutic | Combination | |
| agent | Index | Effect |
| 235-1 and Cytarabine* | 0.80 | moderate synergism |
| 235-1 and Docetaxel* | 0.71 | moderate synergism |
| 235-1 and Paclitaxel* | 0.86 | slight synergism |
| 235-1 and JQ-1*** | 0.42 | synergism |
| 235-1 and I-BET 762*** | 0.56 | synergism |
| 265-1 and Cytarabine** | 1.33 | moderate antagonism |
| 265-1 and Docetaxel** | 0.86 | slight synergism |
| 265-1 and Paclitaxel** | 0.97 | nearly additive |
| 265-1 and JQ-1*** | 0.60 | synergism |
| 265-1 and I-BET 762*** | 0.70 | moderate synergism |
| 268-1 and Cytarabine* | 1.46 | antagonism |
| 268-1 and Docetaxel* | 1.51 | antagonism |
| 268-1 and Paclitaxel* | 1.18 | slight antagonism |

*n = 3
**n = 2
***n = 1

TABLE 10
HAT inhibitors 235-1, 265-1 and 268-1 in combination with
chemotherapeutic agents and/or Bromodomain inhibitors on
LNCaP prostate cancer cells

| Combination of HAT inhibitor | | |
|---|---|---|
| and indicated chemotherapeutic | Combination | |
| agent | Index | Effect |
| 235-1 and Cytarabine* | 0.94 | nearly additive |
| 235-1 and Docetaxel* | 0.79 | moderate synergism |
| 235-1 and Paclitaxel* | 0.83 | moderate synergism |
| 235-1 and JQ-1*** | 1.13 | slight antagonism |
| 235-1 and I-BET 762*** | 1.31 | moderate antagonism |
| 265-1 and Cytarabine** | 0.68 | synergism |
| 265-1 and Docetaxel** | 0.71 | moderate synergism |
| 265-1 and Paclitaxel** | 0.80 | moderate synergism |
| 265-1 and JQ-1*** | 1.20 | slight antagonism |
| 265-1 and I-BET 762*** | 1.17 | slight antagonism |
| 268-1 and Cytarabine* | 0.62 | synergism |
| 268-1 and Docetaxel* | 0.62 | synergism |
| 268-1 and Paclitaxel* | 0.83 | moderate synergism |

*n = 3
**n = 2
***n = 1

TABLE 11
HAT inhibitors 235-1, 265-1 and 268-1 in combination with
chemotherapeutic agents and/or Bromodomain inhibitors on
PC-3 prostate cancer cells

| Combination of HAT inhibitor | | |
|---|---|---|
| and indicated | | |
| chemotherapeutic agent | Combination Index | Effect |
| 235-1 and Cytarabine* | 0.67 | synergism |
| 235-1 and Docetaxel* | 1.12 | slight antagonism |
| 235-1 and Paclitaxel* | 0.84 | moderate synergism |
| 235-1 and JQ-1*** | 0.46 | synergism |
| 235-1 and I-BET 762*** | 0.58 | synergism |
| 265-1 and Cytarabine** | 0.69 | synergism |
| 265-1 and Docetaxel** | 1.14 | slight antagonism |
| 265-1 and Paclitaxel** | 0.92 | nearly additive |
| 265-1 and JQ-1*** | 0.46 | synergism |
| 265-1 and I-BET 762*** | 0.60 | synergism |
| 268-1 and Cytarabine* | 0.55 | synergism |
| 268-1 and Docetaxel* | 1.19 | slight antagonism |
| 268-1 and Paclitaxel* | 1.10 | slight antagonism |

*n = 3
**n = 2
***n = 1

### Radiometric SPA Histone Acetyltransferase Activity Assay

Reactions were performed in a 40 μL volume using an assay buffer containing 100 mM HEPES, pH 7.9, 80 M EDTA, 40 ug/mL BSA, 100 mM KCl, 1 mM DTT, and 0.01% triton X-100. Each compound of interest was dissolved in DMSO and dispensed at 50 nL by a Labcyte Echo (Labcyte, Sunnyvale, Calif.) into polypropylene V bottom 300 uL reaction plates (Greiner) in 3-fold serial dilutions from 50 μM to 0.00075 μM. Compounds were pre-incubated with 20 μL of p300 enzyme (Abbvie) at 10 nM for 30 minutes at room temperature. The reaction was initiated by the addition 20 μL of a biotinylated synthetic Histone H4 Peptide (Anaspec AS-65097) at 25 μM and a mix of 0.5 μM cold acetyl coenzyme A (Moravek MT896H) and 0.3 uM tritiated acetyl coenzyme A (Sigma A2056). The reaction was terminated with the addition of 140 μL of 0.5N aqueous HCl after one hour incubation at room temperature. The reaction contents were transferred to a 96 well streptavidin and scintillant coated microplate (Perkin Elmer SMP103A), incubated for 1 hr, and counted in a Top Count (Perkin Elmer) microplate scintillation counter at one minute per well. IC50 values were generated by analyzing counts per minute using Assay Explorer software (Accelrys). Results of representative Examples can be found in Table 12.

TABLE 12

| | IC50 |
|---|---|
| Example | (μM) |
| 281 | 36.7 |
| 282 | 1.12 |
| 283 | 9.3 |
| 284 | 1.45 |
| 285 | 2.38 |
| 286 | 2.22 |
| 287 | 1.35 |
| 288 | 0.479 |
| 289 | >50 |
| 290 | 1.5 |
| 291 | 0.826 |
| 292 | 0.878 |
| 293 | 1.35 |
| 294 | >50 |
| 295 | >50 |
| 296 | >50 |
| 297 | 0.439 |
| 298 | 0.361 |
| 299 | 0.479 |
| 300 | 0.18 |
| 301 | >50 |
| 302 | 24.5 |
| 303 | 22.8 |
| 304 | 37.7 |
| 305 | 32 |
| 306 | 13.8 |
| 307 | 35.7 |
| 308 | >50 |
| 309 | 0.441 |
| 310 | 2.37 |
| 311 | 0.219 |
| 312 | 1.1 |
| 313 | 1.06 |
| 314 | 0.508 |
| 315 | 1.39 |
| 316 | 1.68 |
| 317 | 1.25 |
| 318 | 1.88 |
| 319 | 0.829 |
| 320 | 1.26 |
| 321 | 16.8 |
| 322 | 0.0298 |
| 323 | >50 |
| 324 | 2.86 |
| 325 | 1.16 |
| 326 | 11.5 |
| 327 | 0.75 |
| 328 | 0.637 |
| 329 | 0.463 |
| 330 | 0.337 |
| 331 | 0.466 |
| 332 | 0.876 |
| 333 | 0.492 |
| 334 | 0.73 |
| 335 | 2.03 |
| 336 | 0.622 |
| 337 | 2.55 |
| 338 | 4.67 |
| 339 | 2.88 |
| 340 | 0.55 |
| 341 | 0.339 |
| 342 | 1.19 |
| 343 | 0.959 |
| 344 | 0.426 |
| 345 | 2.87 |
| 346 | >50 |
| 347 | 0.0912 |
| 348 | 30.0779 |
| 349 | 1.14 |
| 350 | 7.76 |
| 351 | 2.85 |
| 352 | 1.83 |
| 353 | 0.905 |
| 354 | 1.97 |
| 355 | 2.24 |
| 356 | 23.41666 |
| 357 | 6.63 |
| 358 | 1.53 |
| 359 | >50 |
| 360 | 3.88 |
| 361 | 28.6 |
| 362 | >50 |
| 363 | >50 |
| 364 | 5.3 |
| 365 | >50 |
| 366 | 0.349 |
| 367 | >50 |
| 368 | 2.77 |
| 369 | 37.6 |
| 370 | >50 |
| 371 | >50 |
| 372 | >50 |
| 373 | >50 |
| 374 | >50 |
| 375 | >50 |
| 376 | >50 |
| 377 | 0.18 |
| 378 | 0.172 |
| 379 | 0.481 |
| 380 | >50 |
| 381 | >50 |
| 382 | 0.494 |
| 383 | 0.754 |
| 384 | 34.2 |
| 385 | 1.54 |
| 386 | 1.13 |
| 387 | 3.6 |
| 388 | 0.604 |
| 389 | 3.2 |
| 390 | 4.268032 |
| 391 | 9.39206 |
| 392 | 5.54 |
| 393 | 0.62 |
| 394 | 0.534 |
| 395 | 0.443 |
| 396 | 0.255 |
| 397 | 0.738 |
| 398 | 0.286 |
| 399 | 0.185 |
| 400 | 1.14 |
| 401 | >50 |
| 402 | 1.41 |
| 403 | 3.25 |
| 404 | 1.23 |
| 405 | >50 |
| 406 | >50 |
| 407 | >50 |
| 408 | >50 |
| 611 | 2.166749 |
| 955 | 19.53049 |
| 957 | 1.76 |
| 960 | >50 |
| 961 | >50 |
| 963 | >50 |
| 965 | >50 |
| 966 | >50 |
| 967 | >50 |
| 969 | >50 |
| 972 | >50 |
| 975 | >50 |
| 976 | 6.42 |
| 977 | >50 |
| 978 | 26.8 |
| 979 | >50 |
| 980 | 7.32 |
| 984 | 1.52 |
| 985 | 2.78 |
| 986 | 21.7 |
| 988 | 2.01 |

### TR-FRET Histone Acetyltransferase Activity Assay

Reactions were performed in a 10 μL volume using an assay buffer containing 100 mM HEPES, pH 7.9, 80 μM EDTA, 40 ug/mL BSA, 100 mM KCl, 1 mM DTT, 0.01% triton X-100. Each compound of interest was dissolved in DMSO and dispensed at 50 nL by a Labcyte Echo (Labcyte, Sunnyvale, Calif.) into white 384 well low-volume plates (Perkin Elmer 6008289) in 3-fold serial dilutions from 50 μM to 0.00075 μM. Compounds were pre-incubated with 5 μL of p300 enzyme (Abbvie) at 20 nM for 30 minutes at room temperature. The reaction was initiated by the addition of 5 μL of a biotinylated synthetic Histone H4 peptide (Anaspec AS-65097) at 2 μM and acetyl coenzyme A (Sigma-Aldrich A2056) at 2 μM. Following a 1 hour incubation at room temperature in a humidified chamber the reaction was terminated by the addition of 10 μL of 3 nM LANCE Ultra Europium-anti-acetyl-Histone H4 Lysine antibody (Perkin Elmer TRF0412-D), 900 nM LANCE Ultra ULight-Streptavidin (Perkin Elmer TRF0102-D) and 20 M of the lead compound in LANCE Detection Buffer (PerkinElmer CR97-100). TR-FRET measurements were obtained using a Perkin Elmer Envision with laser excitation at 335 nm and emission at 665 nm and 620 nm. IC50 values were generated by analyzing TR-FRET ratios (665 nm/620 nm) using Assay Explorer software (Accelrys). Results of representative Examples can be found in Table 13.

TABLE 13

| | IC50 |
|---|---|
| Example | (μM) |
| 283 | 4.05401 |
| 302 | 25.3002 |
| 307 | >50 |
| 313 | 0.973622 |
| 321 | 25.99846 |
| 322 | 0.027712 |
| 337 | 3.17 |
| 348 | 26.75388 |
| 364 | 9.21 |
| 377 | 0.133079 |
| 409 | 4.795456 |
| 410 | 45.2 |
| 411 | 1.636215 |
| 412 | >50 |
| 413 | 1.405728 |
| 414 | 0.580134 |
| 415 | 0.249487 |
| 416 | 0.182863 |
| 417 | 0.793127 |
| 418 | 2.757064 |
| 419 | 0.657034 |
| 420 | 2.619523 |
| 421 | 0.640873 |
| 422 | 0.431518 |
| 423 | 1.396209 |
| 424 | 12.24908 |
| 425 | >50 |
| 426 | 35.7 |
| 427 | 1.242739 |
| 428 | 1.16 |
| 429 | 1.22 |
| 430 | 0.374 |
| 431 | 0.242 |
| 432 | 0.349 |
| 433 | 0.826 |
| 434 | 0.311 |
| 435 | >50 |
| 436 | >50 |
| 437 | >50 |
| 438 | >50 |
| 439 | >50 |
| 440 | >50 |
| 441 | >50 |
| 442 | >50 |
| 443 | 28.4 |
| 444 | >50 |
| 445 | >50 |
| 446 | >50 |
| 447 | >50 |
| 448 | >50 |
| 449 | >50 |
| 450 | 5.5 |
| 451 | >50 |
| 452 | >50 |
| 453 | >50 |
| 454 | 1.35 |
| 455 | >50 |
| 456 | 0.551 |
| 457 | >50 |
| 458 | >50 |
| 459 | >50 |
| 460 | 4.28 |
| 461 | 6 |
| 462 | >50 |
| 463 | >50 |
| 464 | >50 |
| 465 | >50 |
| 466 | >50 |
| 467 | >50 |
| 468 | 17.9 |
| 469 | >50 |
| 470 | 28.1 |
| 471 | >50 |
| 472 | >50 |
| 473 | >50 |
| 474 | >50 |
| 475 | >50 |
| 476 | >50 |
| 477 | 12.1 |
| 478 | >50 |
| 479 | >50 |
| 480 | >50 |
| 481 | >50 |
| 482 | >50 |
| 483 | >50 |
| 484 | >50 |
| 485 | >50 |
| 486 | 4.7 |
| 487 | 21.6 |
| 488 | 0.0665 |
| 489 | 0.387 |
| 490 | 0.79 |
| 491 | >50 |
| 492 | 4.03 |
| 493 | >50 |
| 494 | 0.519 |
| 495 | 2.05 |
| 496 | 1.84 |
| 497 | 4.71 |
| 498 | 1.21 |
| 499 | 12.6 |
| 500 | 0.181 |
| 501 | 0.637 |
| 502 | 0.336 |
| 503 | 3.43 |
| 504 | 2.6 |
| 505 | 3.07 |
| 506 | 1.59 |
| 507 | 0.361 |
| 508 | 0.766435 |
| 509 | 0.580034 |
| 510 | 0.081679 |
| 511 | 2.35 |
| 512 | 1.275617 |
| 513 | 1.677021 |
| 514 | 0.052027 |
| 515 | >50 |
| 516 | 13.28157 |
| 517 | 8.324458 |
| 518 | 25.5873 |
| 519 | 1.16 |
| 520 | 2.04 |
| 521 | 1.72 |
| 522 | 24 |
| 523 | >50 |
| 524 | >50 |
| 525 | >50 |
| 526 | 0.524 |
| 527 | >50 |
| 528 | >50 |
| 529 | 4.69 |
| 530 | 18.9 |
| 531 | >50 |
| 532 | >50 |
| 533 | >50 |
| 534 | 14.9 |
| 535 | >50 |
| 536 | >50 |
| 538 | 45.6 |
| 539 | >50 |
| 542 | >50 |
| 543 | >50 |
| 545 | 14.4 |
| 546 | >50 |
| 547 | 43.8 |
| 548 | 0.506 |
| 549 | 7.98 |
| 550 | 14.5 |
| 551 | 6.24 |
| 552 | 10.4 |
| 553 | 5.36 |
| 554 | 2.71 |
| 555 | 3.96 |
| 556 | >50 |
| 557 | >50 |
| 558 | 13.9 |
| 559 | 3.68 |
| 560 | 2.63 |
| 561 | 1.8 |
| 562 | 0.77 |
| 563 | 0.022704 |
| 564 | 0.184 |
| 565 | 0.581 |
| 566 | 0.61 |
| 567 | 0.506 |
| 568 | 1.12 |
| 569 | 0.108 |
| 570 | 0.103 |
| 571 | 0.255 |
| 572 | 1.75 |
| 573 | 25.3 |
| 574 | 50 |
| 575 | 1.81 |
| 576 | 15.9 |
| 577 | 0.883 |
| 578 | 16.1 |
| 579 | 0.259 |
| 580 | 0.502 |
| 581 | >50 |
| 582 | 0.106 |
| 583 | 0.791 |
| 584 | 0.541 |
| 585 | 0.844 |
| 586 | 0.867 |
| 587 | 9.84 |
| 588 | >50 |
| 589 | 0.189 |
| 590 | 0.64 |
| 591 | 1.92 |
| 592 | 0.845 |
| 593 | 0.433 |
| 594 | 1.3 |
| 595 | 14.7 |
| 596 | 6.29 |
| 597 | 0.0566 |
| 598 | 0.177 |
| 599 | 0.27 |
| 600 | 0.322 |
| 601 | 0.443 |
| 602 | 0.458 |
| 603 | 0.774 |
| 604 | 0.877 |
| 605 | 1.05 |
| 606 | 1.52 |
| 607 | 3.78 |
| 608 | 24.4 |
| 609 | 0.659 |
| 610 | 0.571976 |
| 611 | 1.71 |
| 613 | 0.0427 |
| 614 | 6.13 |
| 615 | 0.299 |
| 616 | 0.49 |
| 617 | 0.119 |
| 618 | 0.448 |
| 619 | 31.9 |
| 620 | 0.883 |
| 621 | 0.061 |
| 622 | 0.826995 |
| 623 | 0.574 |
| 624 | 3.471786 |
| 625 | >50 |
| 626 | 1.3 |
| 627 | 1.49 |
| 628 | >50 |
| 629 | 0.433 |
| 630 | >50 |
| 631 | 0.808 |
| 632 | 0.738 |
| 633 | 4.21 |
| 634 | 5.66 |
| 635 | 47.6 |
| 636 | >50 |
| 637 | >50 |
| 638 | 14.4 |
| 639 | 0.387 |
| 640 | 3.96 |
| 641 | 3.52 |
| 642 | 2.71 |
| 643 | 0.341 |
| 644 | 2.91 |
| 645 | 21.4 |
| 646 | 15.1 |
| 647 | 12.3 |
| 648 | 19.3 |
| 649 | 37.4 |
| 650 | 0.0628 |
| 651 | 0.318 |
| 652 | 15.1 |
| 653 | 1.05 |
| 654 | 0.384 |
| 655 | 6.54 |
| 656 | 1.34 |
| 657 | 0.584808 |
| 658 | 8.41 |
| 659 | 0.614 |
| 660 | 14.3 |
| 661 | 0.613 |
| 662 | 7.23 |
| 663 | 0.853 |
| 664 | 0.877 |
| 665 | 3.63 |
| 666 | 1.49 |
| 667 | 0.301 |
| 668 | 0.883826 |
| 669 | 1.154773 |
| 670 | 2.85 |
| 671 | 0.732 |
| 672 | 0.795 |
| 673 | 0.222 |
| 674 | 1.48 |
| 675 | 3.33 |
| 676 | 4.59 |
| 677 | 1.22 |
| 678 | 0.032578 |
| 679 | 0.151 |
| 680 | 2.32 |
| 681 | 1.39 |
| 682 | 0.0653 |
| 683 | 0.0757 |
| 684 | 0.715 |
| 685 | 0.842 |
| 686 | 0.597 |
| 687 | 0.374 |
| 688 | 12.6 |
| 689 | 0.36 |
| 690 | 0.0487 |
| 691 | 7.06 |
| 692 | 2.74 |
| 693 | 0.0209 |
| 694 | 1.68 |
| 695 | 1.58 |
| 696 | 0.453 |
| 697 | 0.402 |
| 698 | 0.739 |
| 699 | 0.554 |
| 700 | 0.0106 |
| 701 | 0.0468 |
| 702 | 0.408 |
| 703 | 0.549 |
| 704 | 0.072 |
| 705 | 1.05 |
| 706 | 0.0388 |
| 707 | 0.528 |
| 708 | 23.9 |
| 709 | 0.574 |
| 710 | 0.445 |
| 711 | 6.31 |
| 712 | 5.44 |
| 713 | 0.0966 |
| 714 | 0.249 |
| 715 | 0.0188 |
| 716 | 0.837 |
| 717 | 0.0316 |
| 718 | 0.777 |
| 719 | 29.6 |
| 720 | 0.43919 |
| 721 | 0.71618 |
| 722 | 0.946099 |
| 723 | 0.663491 |
| 724 | 0.558 |
| 725 | 0.143 |
| 726 | 0.817 |
| 727 | 5.35 |
| 728 | 0.291 |
| 729 | 13.5 |
| 730 | 0.0231 |
| 731 | 1.46 |
| 732 | 1.58 |
| 733 | 1.53 |
| 734 | 0.665 |
| 735 | 0.0255 |
| 736 | 15 |
| 737 | 2.58 |
| 738 | 0.32 |
| 739 | 0.0586 |
| 740 | 0.0666 |
| 741 | 0.0933 |
| 742 | 0.154 |
| 743 | 1.03 |
| 744 | 0.0729 |
| 745 | 0.0408 |
| 746 | 0.0457 |
| 747 | 0.112 |
| 748 | 0.111 |
| 749 | 0.296 |
| 750 | 4.9 |
| 751 | 11.4 |
| 752 | 0.243 |
| 753 | 8.93 |
| 754 | 0.707 |
| 755 | 0.811 |
| 756 | 5.87 |
| 757 | 0.169 |
| 759 | 0.0169 |
| 760 | 0.172 |
| 761 | 8.73 |
| 762 | 4.47 |
| 763 | 0.206 |
| 764 | 1.32 |
| 765 | 30.5 |
| 766 | 0.0326 |
| 767 | >50 |
| 768 | 2.85 |
| 769 | 0.334 |
| 770 | 0.421 |
| 771 | 7.78 |
| 772 | 2.37 |
| 773 | 0.0518 |
| 774 | 3.74 |
| 775 | 22.5 |
| 776 | 36.1 |
| 777 | 5.55 |
| 778 | 1.09 |
| 779 | 0.0374 |
| 780 | 0.124 |
| 781 | 0.125 |
| 782 | 2.68 |
| 783 | 0.168 |
| 784 | 0.196 |
| 785 | 3.92 |
| 786 | 7.9 |
| 787 | 1.91 |
| 788 | 2.4 |
| 789 | 0.0187 |
| 790 | >50 |
| 791 | 0.195 |
| 792 | 0.143 |
| 793 | 0.186 |
| 794 | 0.291 |
| 795 | 1.48 |
| 796 | 2.08 |
| 797 | 0.447 |
| 798 | 39.5 |
| 799 | 3.08 |
| 800 | 0.0275 |
| 801 | 0.0633 |
| 802 | 0.026 |
| 803 | 0.0295 |
| 804 | >50 |
| 805 | 24.4 |
| 806 | 5.58 |
| 807 | 3.19 |
| 808 | 20.4 |
| 809 | >50 |
| 810 | 0.158 |
| 811 | 17.9 |
| 812 | >50 |
| 813 | 39.6 |
| 814 | 0.986 |
| 815 | 1.24 |
| 816 | 0.0121 |
| 817 | 0.221 |
| 818 | 0.373 |
| 819 | 1.04 |
| 820 | 49.2 |
| 821 | 47.1 |
| 822 | >50 |
| 823 | 5.92 |
| 824 | 2.75 |
| 825 | 10.6 |
| 826 | 2.59 |
| 827 | 45.1 |
| 828 | 20.8 |
| 829 | 0.584 |
| 830 | 23 |
| 831 | 0.719 |
| 832 | 12.6 |
| 833 | 0.259 |
| 834 | 0.0273 |
| 835 | >50 |
| 836 | 0.0373 |
| 837 | 5.29 |
| 838 | 0.0541 |
| 839 | 0.0879 |
| 840 | 0.0195 |
| 841 | 0.504 |
| 842 | 0.375 |
| 843 | 0.0598 |
| 844 | 0.974 |
| 845 | 0.0197 |
| 846 | 0.205 |
| 847 | 6.69 |
| 848 | 6.09 |
| 849 | 21.4 |
| 850 | 0.0359 |
| 851 | 0.306 |
| 852 | 3.17 |
| 853 | >50 |
| 854 | 0.141 |
| 855 | 0.0179 |
| 856 | 0.0242 |
| 857 | 2.11 |
| 858 | 10.3 |
| 859 | 0.78 |
| 860 | 0.948 |
| 861 | 0.0169 |
| 862 | >50 |
| 863 | 0.562 |
| 864 | 0.0557 |
| 865 | 0.523 |
| 866 | 21.9 |
| 867 | 0.748 |
| 868 | 0.0753 |
| 869 | 0.162 |
| 870 | 0.0107 |
| 871 | 0.0246 |
| 872 | 6.45 |
| 873 | 8.28 |
| 874 | 0.122 |
| 875 | 0.0179 |
| 876 | 4.47 |
| 877 | >50 |
| 878 | 9.55 |
| 879 | >50 |
| 880 | 8.75 |
| 881 | 0.303 |
| 882 | 0.599 |
| 883 | 0.00607 |
| 884 | 0.00922 |
| 885 | 0.0147 |
| 886 | 0.0149 |
| 887 | 0.107 |
| 888 | >50 |
| 889 | 1.56 |
| 890 | 2.82 |
| 891 | 2.14 |
| 892 | 0.0208 |
| 893 | 0.48 |
| 894 | >50 |
| 895 | >50 |
| 896 | 0.604 |
| 897 | >50 |
| 898 | 0.149 |
| 899 | 1.3 |
| 900 | 0.978 |
| 901 | >50 |
| 902 | >50 |
| 903 | 0.842 |
| 904 | >50 |
| 905 | >50 |
| 906 | >50 |
| 907 | 0.905 |
| 908 | 0.00984 |
| 909 | 0.05 |
| 910 | 0.129 |
| 911 | 8.15 |
| 912 | >50 |
| 913 | 21.3 |
| 914 | 0.0505 |
| 915 | 6.22 |
| 916 | 1.42 |
| 917 | 0.048 |
| 918 | 31.5 |
| 919 | 0.432 |
| 920 | 0.3 |
| 921 | 0.00553 |
| 922 | 0.522 |
| 923 | 6.06 |
| 924 | 0.594 |
| 925 | 0.061 |
| 926 | 23 |
| 927 | 0.814 |
| 928 | 0.0123 |
| 929 | 20.2 |
| 930 | 33.2 |
| 931 | 0.966 |
| 932 | 9.01 |
| 933 | 0.263 |
| 934 | 0.134 |
| 935 | 1.25 |
| 936 | 0.415 |
| 937 | 0.233 |
| 938 | 0.0106 |
| 939 | 0.0533 |
| 940 | >50 |
| 941 | >50 |
| 942 | 0.128 |
| 943 | 0.101 |
| 944 | 1.89 |
| 945 | 0.164 |
| 946 | 0.027 |
| 947 | 0.109 |
| 948 | 8.78 |
| 949 | 0.515 |
| 950 | 16.6 |
| 951 | 0.981 |
| 952 | 0.0371 |
| 953 | 0.426 |
| 954 | 0.0185 |
| 989 | 0.0324 |
| 990 | 0.0251 |
| 991 | 0.179 |
| 992 | 0.228 |
| 993 | >50 |
| 994 | 2.61 |
| 995 | 0.0683 |
| 996 | 0.0949 |
| 997 | 0.604 |
| 998 | 0.0183 |
| 999 | 0.0417 |
| 1000 | 0.493498 |
| 1001 | 0.286967 |
| 1002 | 28.8 |
| 1003 | 8.57 |
| 1004 | 2.67 |
| 1005 | 1.4 |
| 1006 | 0.312 |
| 1007 | 2.08 |
| 1008 | 1.1 |
| 1009 | >50 |
| 1010 | >50 |
| 1011 | 2.65 |
| 1012 | 1.18 |
| 1013 | 0.12 |
| 1014 | 0.0596 |
| 1015 | 1.23 |
| 1016 | 0.0925 |
| 1017 | 0.951 |
| 1018 | 36 |
| 1019 | 4.5 |
| 1020 | 0.108 |
| 1021 | 0.0686 |
| 1022 | >50 |
| 1023 | 4.28 |
| 1024 | 4.28 |
| 1025 | 1.15 |
| 1026 | 0.401 |
| 1027 | 0.0294 |
| 1029 | 27.8 |
| 1030 | 1.24 |
| 1031 | 0.0168 |
| 1032 | 0.0897 |
| 1033 | 0.125 |

### p300 LnCap Proliferation Assay

LnCap-FGC cells were plated into white-walled 96 well plates (Costar 3610) at 5,000 and 1,500 cells per well, respectively in 100 μl of RPMI 1640 supplemented with 10% fetal bovine serum (FBS). After the cells had adhered for 24 hours at 37° C.+5% CO₂, compounds were added in a 7-point half-log dose response for a final volume of 200 μl per well. After 5 days at 37° C., media was removed (Biotek EL406) leaving 50 μl per well. An equal volume of Cell Titer Glo regeant (Promega G8462) was added and it was incubated for 10 minutes at room temperature. Luminescence was then read using Biotek Synergy. IC₅₀ values were calculated using a sigmoidal fit of the concentration/inhibition response curves, and are shown in Table 13A.

TABLE 13A

| | IC50 |
|---|---|
| Example | (μM) |
| 117-1 | 1.172323 |
| 121-1 | 0.284 |
| 147-1 | 1.218975 |
| 172-1 | 0.506 |
| 175-1 | 0.346 |
| 181-1 | 0.353338 |
| 183-1 | 1.38 |
| 184-1 | 1.476889 |
| 202-1 | 0.66 |
| 203-1 | 0.361 |
| 216-1 | 0.277 |
| 219-1 | 0.750804 |
| 227-1 | 0.263 |
| 228-1 | 0.379 |
| 233-1 | 0.243 |
| 235-1 | 0.229 |
| 235-2 | 1.78 |
| 261-2 | 0.532546 |
| 265-1 | 0.234256 |
| 266-1 | 0.394073 |
| 266-2 | 3.72874 |
| 267-1 | 0.282 |
| 268-3 | 0.174805 |
| 269-1 | 0.121417 |
| 270-1 | 0.705521 |
| 271-1 | 0.427873 |
| 272-1 | 0.15302 |
| 273-1 | 0.306 |
| 274-1 | 0.706399 |
| 275-1 | 0.644981 |
| 288 | 0.287 |
| 297 | 0.275 |
| 298 | 0.408 |
| 299 | 2.25 |
| 300 | 7.454514 |
| 309 | 0.624 |
| 311 | 0.212471 |
| 322 | 0.329967 |
| 329 | 1.07 |
| 330 | 0.733 |
| 331 | 0.503 |
| 333 | 0.854 |
| 341 | 0.965 |
| 344 | 0.292536 |
| 347 | 0.95869 |
| 349 | 1.54 |
| 353 | 0.682233 |
| 366 | 1.294527 |
| 368 | 4.182822 |
| 377 | 0.411148 |
| 378 | 0.486062 |
| 379 | 2.965755 |
| 382 | 1.265543 |
| 383 | 0.822296 |
| 388 | 2.714959 |
| 393 | 0.651 |
| 394 | 0.622 |
| 395 | 0.823 |
| 396 | 0.443 |
| 397 | 1.91 |
| 398 | 0.490075 |
| 399 | 0.367388 |
| 414 | 0.437073 |
| 415 | 0.331 |
| 416 | 0.182757 |
| 419 | 1.05 |
| 421 | 0.423791 |
| 422 | 0.639 |
| 430 | 0.29026 |
| 431 | 0.22771 |
| 432 | 0.383279 |
| 433 | 3.56 |
| 434 | 0.677008 |
| 456 | 1.104203 |
| 488 | 0.506062 |
| 489 | 3.882989 |
| 490 | 1.78 |
| 500 | 2.0291 |
| 507 | 0.561 |
| 510 | 0.443161 |
| 514 | 0.171432 |
| 526 | 1.808452 |
| 562 | 0.424476 |
| 563 | 0.119877 |
| 564 | 2.56101 |
| 565 | 0.667 |
| 566 | 1.44 |
| 567 | 0.51 |
| 569 | 0.299204 |
| 570 | 0.175 |
| 571 | 8.884256 |
| 577 | 1.19 |
| 579 | 0.393004 |
| 580 | >10 |
| 582 | 1.104083 |
| 583 | 2.01 |
| 584 | 0.634 |
| 585 | 1.88 |
| 586 | 2.69 |
| 589 | 0.644 |
| 590 | 1.51 |
| 592 | 0.574701 |
| 593 | 1.66 |
| 597 | 0.156783 |
| 598 | 0.173508 |
| 599 | 0.357 |
| 600 | 0.347633 |
| 601 | 1.929508 |
| 602 | 0.779 |
| 603 | 0.746 |
| 604 | 1.65 |
| 609 | 0.952 |
| 610 | 0.77 |
| 613 | 0.76642 |
| 615 | 2.611666 |
| 616 | 4.359197 |
| 617 | 0.286 |
| 618 | 0.566 |
| 620 | 1.29 |
| 621 | 0.269557 |
| 622 | 1.8 |
| 623 | 2.24 |
| 629 | 2.67 |
| 631 | 2.55 |
| 632 | 3.61 |
| 639 | 0.636428 |
| 643 | 0.598 |
| 650 | 0.072542 |
| 651 | 0.196667 |
| 654 | 0.507 |
| 657 | 0.902618 |
| 659 | >10 |
| 661 | 0.2965 |
| 663 | 0.383 |
| 664 | 0.598 |
| 667 | 1.797053 |
| 668 | 0.722613 |
| 671 | 5.146576 |
| 672 | 0.413 |
| 673 | 0.32 |
| 678 | >10 |
| 679 | 0.690352 |
| 682 | 0.215917 |
| 683 | 0.303322 |
| 684 | 0.677385 |
| 685 | 4.960081 |
| 686 | 1.886372 |
| 687 | >10 |
| 689 | 0.60425 |
| 690 | 0.255186 |
| 693 | 0.071517 |
| 696 | 1.890661 |
| 697 | 0.768375 |
| 698 | 1.39 |
| 699 | 0.318 |
| 700 | 0.016733 |
| 701 | 0.120665 |
| 702 | 0.25 |
| 703 | 2.27 |
| 704 | 0.357994 |
| 706 | 0.081035 |
| 707 | 0.649 |
| 709 | 0.761 |
| 710 | 0.819 |
| 713 | 0.607602 |
| 714 | 0.376 |
| 715 | 0.0148 |
| 716 | 1.461267 |
| 717 | 0.129256 |
| 718 | 0.325 |
| 720 | 0.382225 |
| 721 | 0.374492 |
| 722 | 0.515345 |
| 723 | 0.47449 |
| 724 | 4.616362 |
| 725 | 0.294 |
| 726 | 0.709 |
| 728 | 0.754481 |
| 730 | >10 |
| 734 | 0.625 |
| 735 | 0.04694 |
| 738 | 0.333 |
| 739 | 0.248375 |
| 740 | 0.283972 |
| 741 | 0.389245 |
| 742 | 0.2548 |
| 744 | 0.426596 |
| 745 | 0.204118 |
| 746 | 0.146096 |
| 747 | 0.196962 |
| 748 | 0.202583 |
| 749 | 0.408 |
| 752 | 0.334 |
| 753 | 7.195 |
| 754 | 0.523 |
| 755 | 0.222 |
| 757 | 0.140993 |
| 759 | 0.06261 |
| 760 | 1.35 |
| 763 | 0.395 |
| 766 | 0.050677 |
| 769 | 0.256 |
| 770 | 0.637385 |
| 773 | 0.598 |
| 774 | 8.32 |
| 779 | 2.071232 |
| 780 | >10 |
| 781 | >10 |
| 783 | >10 |
| 784 | 1.183119 |
| 789 | 0.019494 |
| 791 | 0.282 |
| 792 | 0.386 |
| 793 | 0.232 |
| 794 | 0.473 |
| 797 | 0.313263 |
| 800 | 0.041897 |
| 801 | 0.107079 |
| 802 | 0.054047 |
| 803 | 0.028114 |
| 814 | 0.711 |
| 816 | >10 |
| 817 | 0.229 |
| 818 | 1.396424 |
| 829 | 0.886121 |
| 831 | 0.433 |
| 833 | >10 |
| 834 | >10 |
| 836 | >10 |
| 838 | >10 |
| 839 | >10 |
| 840 | 8.95 |
| 841 | 2.21 |
| 842 | 0.856 |
| 843 | 0.533 |
| 844 | >10 |
| 845 | <0.01 |
| 846 | >10 |
| 850 | 0.2 |
| 851 | 0.351 |
| 854 | >10 |
| 855 | 3.08 |
| 856 | >10 |
| 857 | 2.6 |
| 859 | 11.6 |
| 860 | 12.6 |
| 861 | 0.0356 |
| 862 | >30 |
| 863 | 0.891 |
| 865 | >30 |
| 866 | 6.01 |
| 867 | 2.91 |
| 868 | >30 |
| 870 | 0.049 |
| 871 | 0.0927 |
| 872 | 12.1 |
| 873 | 7.08 |
| 874 | >30 |
| 875 | 0.459 |
| 876 | 4.48 |
| 877 | 5.16 |
| 878 | 1.930844 |
| 879 | 5.11 |
| 880 | 4.51 |
| 881 | 0.662 |
| 882 | 15.5 |
| 883 | 2.42 |
| 884 | 15.5 |
| 885 | 0.558 |
| 886 | 4.54 |
| 887 | 1.42 |
| 888 | 16.79256 |
| 889 | 1.96 |
| 890 | >30 |
| 891 | >30 |
| 892 | 0.0691 |
| 893 | 0.602 |
| 894 | >30 |
| 895 | 28.7 |
| 897 | 4.57 |
| 901 | >30 |
| 902 | >30 |
| 905 | 23 |
| 906 | 2.17 |
| 907 | 1.43 |
| 908 | 0.217 |
| 909 | 5.3 |
| 910 | 1.58 |
| 911 | 20.9 |
| 912 | 20.2 |
| 913 | 15 |
| 914 | 0.0661 |
| 915 | 9.44 |
| 916 | 3.4 |
| 917 | 0.267 |
| 919 | 1.17 |
| 920 | 1.1 |
| 921 | 0.905 |
| 922 | 5.13 |
| 923 | >30 |
| 927 | 4.54 |
| 929 | 13.1 |
| 933 | 5.22 |
| 936 | 15.5 |
| 938 | 3.5 |
| 939 | 6.65 |
| 942 | 13.1 |
| 943 | 0.955 |
| 944 | 3.13 |
| 945 | 1.84 |
| 946 | 0.313 |
| 947 | 0.994 |
| 948 | 24.8 |
| 950 | 6.43 |
| 951 | 10 |
| 952 | 0.31 |
| 953 | 3.1 |
| 954 | 0.183 |
| 989 | 0.243 |
| 990 | 0.348 |
| 991 | 0.99 |
| 992 | 2.26 |
| 994 | 2.44 |
| 996 | 0.558 |
| 997 | 2.3 |
| 998 | 0.114 |
| 999 | 0.124 |
| 1000 | 1.44 |
| 1001 | 1.25 |

### p300H3K27Ac PD Assay

PC-3 cells were plated into collagen coated black-walled viewplates (Perkin Elmer 6005810) at 5,000 cells per well for 24 hours in 100 μl of RPMI 1640 supplemented with 10% fetal bovine serum (FBS), 1× non-essential amino acids and 1 mM final concentration sodium pyruvate. After the cells had adhered for 24 hours at 37° C.+5% CO₂, they were treated with a 8-point half-log dose response of compounds for 3 hours. Cells were then fixed in 2% (final concentration) formaldehyde (Polysciences, Inc. #04018) at room temperature for 10 minutes, washed in PBS (phosphate buffered saline), and then permeabalized in 0.1% Triton X-100 for 10 minutes. Cells were washed 4 times in PBS and then blocked in 1% BSA (bovine serum albumin) fraction V (Gibco 15260-037) in PBS and shook for 1 hour at room temperature. The buffer was removed and 60 μl per well of primary Histone H3K27 Acetyl (H3K27Ac) antibody (Cell Signaling 8173S) diluted in 0.3% BSA in PBS at (1:500 dilution) was added overnight. Cells were washed four times in PBS and then incubated with a mixture of Alexa Fluor488-conjugated goat anti-rabbit IgG antibodies (Life Technologies, #A-11029) and Hoechst 33342 (Life Technologies, #H3570) for 1.5 hours at room temperature. After washing four times in PBS, plates were scanned within 24 hours of processing on a CellInsight using the target activation algorithm acquiring 15 fields per well. Fluorescence intensities were quantified using the average mean intensity function. Background was calculated using the signal of a rabbit isotype control antibody (Cell Signaling 3900S). IC₅₀ values for H3K27Ac inhibition were calculated using a sigmoidal fit of the concentration/inhibition response curves, and are shown in Table 13B.

TABLE 13B

| | H3K27Ac |
|---|---|
| | IC50 |
| Example | (μM) |
| 117-1 | 0.680728 |
| 121-1 | 0.129 |
| 147-1 | 0.797 |
| 172-1 | 0.0419 |
| 175-1 | 0.0965 |
| 181-1 | 0.024012 |
| 183-1 | 0.190958 |
| 184-1 | 0.287 |
| 202-1 | 0.0391 |
| 203-1 | 0.176 |
| 216-1 | 0.0633 |
| 219-1 | 0.471 |
| 227-1 | 0.0927 |
| 228-1 | 0.13 |
| 233-1 | 0.0652 |
| 235-1 | 0.047098 |
| 235-2 | 0.767872 |
| 261-2 | 0.032303 |
| 265-1 | 0.029026 |
| 266-1 | 0.100741 |
| 266-2 | 0.368519 |
| 267-1 | 0.0407 |
| 268-3 | 0.017541 |
| 269-1 | 0.028404 |
| 270-1 | 0.0669 |
| 271-1 | 0.024884 |
| 272-1 | 0.045482 |
| 273-1 | 0.038827 |
| 274-1 | 0.044945 |
| 275-1 | 0.06 |
| 288 | 0.0959 |
| 297 | 0.263488 |
| 298 | 0.0897 |
| 299 | 0.352 |
| 300 | 1.826684 |
| 309 | 0.124 |
| 311 | 0.036304 |
| 322 | 0.0363 |
| 329 | 0.176 |
| 330 | 0.134 |
| 331 | 0.115 |
| 333 | 0.268 |
| 341 | 0.110955 |
| 344 | 0.0888 |
| 347 | 0.219657 |
| 349 | 0.486 |
| 353 | 0.391046 |
| 366 | 0.408 |
| 368 | 2.67 |
| 377 | 0.0595 |
| 378 | 0.0564 |
| 379 | 0.742 |
| 382 | 0.268 |
| 383 | 0.178 |
| 388 | 0.437 |
| 393 | 0.098387 |
| 394 | 0.141 |
| 395 | 0.187 |
| 396 | 0.0398 |
| 397 | 0.506 |
| 398 | 0.142 |
| 399 | 0.0983 |
| 414 | 0.282128 |
| 415 | 0.0896 |
| 416 | 0.072748 |
| 419 | 0.212 |
| 421 | 0.2 |
| 422 | 0.127 |
| 430 | 0.131 |
| 431 | 0.0819 |
| 432 | 0.17508 |
| 433 | 2.09 |
| 434 | 0.258936 |
| 456 | 0.432944 |
| 488 | 0.349969 |
| 489 | 1.343536 |
| 490 | 1.08 |
| 500 | 0.962327 |
| 507 | 0.046583 |
| 510 | 0.146997 |
| 514 | 0.022869 |
| 526 | 0.538331 |
| 562 | 0.110995 |
| 563 | 0.043621 |
| 564 | 0.496339 |
| 565 | 0.323 |
| 566 | 0.939 |
| 567 | 0.211 |
| 569 | 0.081738 |
| 570 | 0.0878 |
| 571 | 3.449725 |
| 577 | 0.44 |
| 579 | 0.239591 |
| 582 | 0.257787 |
| 583 | 1.38 |
| 584 | 0.219 |
| 585 | 1.1 |
| 586 | 1.37 |
| 589 | 0.188 |
| 590 | 0.52 |
| 592 | 0.363874 |
| 593 | 0.444 |
| 597 | 0.027156 |
| 598 | 0.043652 |
| 599 | 0.256 |
| 600 | 0.125124 |
| 601 | 0.37204 |
| 602 | 0.248 |
| 603 | 0.245 |
| 604 | 0.405 |
| 609 | 0.189 |
| 610 | 0.201 |
| 613 | 0.332632 |
| 615 | 2.171129 |
| 616 | 2.382226 |
| 617 | 0.097 |
| 618 | 0.14 |
| 620 | 0.811 |
| 621 | 0.037458 |
| 622 | 0.228 |
| 623 | 0.41 |
| 629 | 0.403 |
| 631 | 0.448 |
| 632 | 0.577 |
| 639 | 0.08081 |
| 643 | 0.0855 |
| 650 | 0.00623 |
| 651 | 0.047238 |
| 654 | 0.0763 |
| 657 | 0.493672 |
| 661 | 0.019446 |
| 663 | 0.136 |
| 664 | 0.128 |
| 667 | 0.624163 |
| 668 | 0.278948 |
| 671 | 0.474834 |
| 672 | 0.148 |
| 673 | 0.119 |
| 678 | >10 |
| 679 | 0.077314 |
| 682 | 0.037 |
| 683 | 0.045456 |
| 684 | 0.213474 |
| 685 | 1.319091 |
| 686 | 0.911032 |
| 687 | >10 |
| 689 | 0.219834 |
| 690 | 0.051685 |
| 693 | 0.017762 |
| 696 | 0.668099 |
| 697 | 0.153274 |
| 698 | 1.05 |
| 699 | 0.146 |
| 700 | 0.00397 |
| 701 | 0.089219 |
| 702 | 0.0658 |
| 703 | 2.18 |
| 704 | 0.196535 |
| 706 | 0.023195 |
| 707 | 0.164 |
| 709 | 0.505 |
| 710 | 0.575 |
| 713 | 0.355725 |
| 714 | 0.236 |
| 715 | 0.004596 |
| 716 | 1.127972 |
| 717 | 0.107331 |
| 718 | 0.198 |
| 720 | 0.15848 |
| 721 | 0.168603 |
| 722 | 0.161988 |
| 723 | 0.241711 |
| 724 | 3.597068 |
| 725 | 0.124 |
| 726 | 0.662 |
| 728 | 0.719856 |
| 730 | 1.878856 |
| 734 | 0.374 |
| 735 | 0.00776 |
| 738 | 0.121 |
| 739 | 0.031142 |
| 740 | 0.054001 |
| 741 | 0.080985 |
| 742 | 0.053185 |
| 744 | 0.088623 |
| 745 | 0.035553 |
| 746 | 0.023461 |
| 747 | 0.052372 |
| 748 | 0.067415 |
| 749 | 0.135 |
| 752 | 0.0803 |
| 753 | 7.66 |
| 754 | 0.131 |
| 755 | 0.0707 |
| 757 | 0.026775 |
| 759 | 0.013348 |
| 760 | 0.712 |
| 763 | 0.24 |
| 766 | 0.008054 |
| 769 | 0.127 |
| 770 | 0.149923 |
| 773 | 0.153 |
| 774 | 5.51 |
| 779 | 0.226455 |
| 780 | 4.467136 |
| 781 | >10 |
| 783 | >10 |
| 784 | 0.834518 |
| 789 | 0.004174 |
| 791 | 0.101 |
| 792 | 0.12 |
| 793 | 0.0888 |
| 794 | 0.159 |
| 797 | 0.07825 |
| 800 | 0.006804 |
| 801 | 0.014946 |
| 802 | 0.008173 |
| 803 | 0.00508 |
| 810 | >10 |
| 814 | 0.224 |
| 816 | 3.752626 |
| 817 | 0.0886 |
| 818 | 0.496387 |
| 829 | 0.276266 |
| 831 | 0.188 |
| 833 | >10 |
| 834 | 0.783119 |
| 836 | 2.713043 |
| 838 | 8.56787 |
| 839 | >10 |
| 840 | 3.014797 |
| 841 | 1.178474 |
| 842 | 0.353 |
| 843 | 0.233 |
| 844 | 4.36 |
| 845 | 0.00349 |
| 850 | 0.03049 |
| 851 | 0.106 |
| 854 | 8.47 |
| 855 | 0.827496 |
| 856 | >10 |
| 860 | >10 |
| 861 | 0.006587 |
| 863 | 0.339 |
| 867 | 1.654992 |
| 868 | >10 |
| 870 | 0.003855 |
| 871 | 0.007431 |
| 872 | 1.17 |
| 873 | 3.36 |
| 875 | 0.131704 |
| 881 | 0.129 |
| 882 | >10 |
| 883 | 1.645327 |
| 884 | 6.588991 |
| 885 | 0.217433 |
| 886 | 2.959865 |
| 887 | 0.056807 |
| 892 | 0.011597 |
| 893 | 0.12 |
| 896 | >10 |
| 907 | 0.469 |
| 908 | 0.030299 |
| 909 | 0.761725 |
| 910 | 0.047046 |
| 914 | 0.0697 |
| 917 | 0.077771 |
| 919 | 0.924 |
| 920 | 0.335 |
| 921 | 0.510529 |
| 922 | 0.570563 |
| 924 | >10 |
| 925 | 2.502798 |
| 927 | 0.622238 |
| 928 | 0.856957 |
| 931 | 1.934838 |
| 933 | 0.194 |
| 936 | 0.917421 |
| 938 | 0.12506 |
| 939 | 1.579715 |
| 942 | 5.499545 |
| 943 | 0.03034 |
| 945 | 0.22775 |
| 947 | 0.0502 |
| 951 | 2.891072 |
| 952 | 0.01819 |
| 953 | 0.240031 |
| 954 | 0.005343 |
| 989 | 0.004269 |
| 990 | 0.005603 |
| 991 | 0.055 |
| 992 | 0.17 |
| 995 | >10 |
| 996 | 0.0383 |
| 997 | 0.373 |
| 998 | 0.003749 |
| 999 | 0.005894 |
| 1000 | 0.091302 |
| 1001 | 0.146 |
| 1006 | 0.2 |
| 1013 | 3.13 |
| 1014 | 0.0441 |
| 1016 | 0.279 |
| 1017 | 1.89 |
| 1021 | 0.036 |

### In Vivo Tumor Models

The effect of p300 inhibitors on tumor growth was evaluated in subcutaneous Kasumi-1 (acute myeloid leukemia), SuDHL-8 (B-cell lymphoma), 22RV1 (prostate), and NCI-H929 (multiple myeloma) xenograft tumors implanted in SCID female mice (Charles Rivers Labs). Briefly, human cancer cells were inoculated subcutaneously into the right hind flank of female SCID mice on study day 0. Administration of p300 inhibitors was initiated at the time of size match. Tumor volume was measured for the duration of the experiment until the mean tumor volume in each group reached an endpoint of ≥2000 mm³ using the following formula; V=L×W²/2. Results can be found in Table 13C. The inhibition of p300 induced significant tumor growth inhibition in multiple xenograft tumor models.

TABLE 13C

| | Kasumi-1 | | SUDHL-1 | | NCI-H929 | | 22Rv1 | |
|---|---|---|---|---|---|---|---|---|
| Example | mg/kg/day | % TGI* | mg/kg/day | % TGI* | mg/kg/day | % TGI* | mg/kg/day | % TGI* |
| 266-1 | 150 | 51 | 150 | 78 | 125 | 70 | 125 | 49 |
| 298 | | | 125 | 57 | 100 | 29 | | |
| 311 | | | 10 | 46 | 10 | 50 | | |
| 700 | | | 20 | 53 | | | 20 | 0 |
| 715 | | | 7.5 | 62 | | | 7.5 | 48 |
| 759 | | | | | | | 7.5 | 47 |

*% TGI (tumor growth inhibition) = (control − treated)/control × 100; calculated on the last day the controls were measured

All of the U.S. patents, U.S. patent application publications, U.S. patent applications, foreign patents, foreign patent applications and non-patent publications referred to in this specification or Application Data sheet are incorporated herein by reference, in their entirety to the extent not inconsistent with the present description.

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. A compound having Formula (IX), 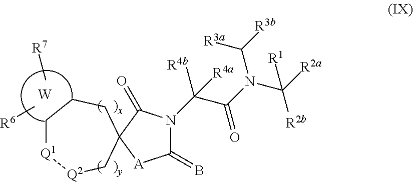
or a stereoisomer, tautomer or pharmaceutically acceptable salt thereof,
wherein [symbol] [symbol]
is —C(R¹⁰)₂—C(R¹⁴)₂—, —O—C(R¹⁴)₂—, —O—C(O)—, —S(O)₂—C(R¹⁴)₂—, S—C(R¹⁴)₂—, —NR⁹—C(O)—, —NR⁹—C(R¹⁴)₂—, —C(R¹⁰)₂—O—, —C(R¹⁰)₂—, or —C(R¹⁰)═C(R¹⁴)—;
A is —NR⁸—, —O—, or —S—;
B is O or NH;
W is arylene or heteroarylene;
R¹ is carbocyclyl or heterocyclyl;
R^{2a} and R^{2b} are each independently H, D, or C₁-C₆ alkyl;
R^{3a} is hydrogen, C(O)NH₂, C₁-C₆ alkyl, aryl, cycloalkyl or heterocyclyl; and
R^{3b} is C₁-C₆ alkyl, aryl, cycloalkyl or heterocyclyl; or
R^{3a} and R^{3b} taken together with the carbon to which they are attached form an arene, cycloalkane, or heterocycle;
R^{4a} and R^{4b} are each independently H, D or C₁-C₆ alkyl;
R⁶ and R⁷ are each independently H, halo, —OH, —CN, —CO₂H, C₁-C₆ alkyl, alkoxy, haloalkoxy, alkoxyalkyl, haloalkoxyalkyl, hydroxylalkyl, hydroxylalkynyl, aryl, cycloalkyl, heterocyclyl, heterocyclylalkyl, heterocyclyloxy, —B(R¹¹)(R¹³), —S(O)ₘR², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, —NHC(═O)NR¹²S(O)₂OR¹², —NHC(═O)NR¹²S(O)₂N(R¹²)₂, —N HC(═S)N(R¹²)₂, —NHC(═N—C≡N)NR¹², —NHC(═N—C≡N)SR¹², or —NHS(O)ₘR¹²;
R⁸ and R⁹ are each independently H or C₁-C₆ alkyl;
R¹⁰, at each occurrence, is independently H, —OH, halo, —CN, —CO₂R¹², —C(═O)NHR¹³, —NHR¹², C₁-C₆ alkyl or alkoxy; or two R¹⁰ taken together form oxo or ═N—OR¹¹;
R¹¹ and R¹³ are each independently H, —OH, or C₁-C₆ alkyl;
R¹², at each occurrence, is independently H, C₁-C₆ alkyl, aryl, cycloalkyl, or heterocyclyl;
R¹⁴, at each occurrence, is independently H or C₁-C₆ alkyl;
m, at each occurrence, is independently 0, 1 or 2; and
x, and y are each independently 0 or 1, wherein x and y are selected such that the sum of x+y is 0 or 1;
with the proviso that R^{3a} and R^{3b} are not cyclopropyl and methyl, respectively, when R¹ and W are each unsubstituted phenyl, A is —NH, x is 0 or 1, y is 0, and [symbol] is —C(R¹⁰)₂—C(R¹⁴)₂—; and
with the proviso that R^{3a} and R^{3b} taken together with the carbon to which they are attached do not form tetrahydrothiophene 1,1-dioxide or tetrahydrothiophene when at least one of R¹ and W is unsubstituted phenyl, and A is —NH.

2. The compound of claim 1, or pharmaceutically acceptable salt thereof, wherein A is —O—;

3. The compound of claim 1, or pharmaceutically acceptable salt thereof, wherein A is —NR⁸; and R⁸ is H.

4. The compound of claim 1, or pharmaceutically acceptable salt thereof, wherein W is arylene.

5. The compound of claim 1, or pharmaceutically acceptable salt thereof, wherein W is phenylene.

6. The compound of claim 5, or pharmaceutically acceptable salt thereof, wherein R^{4a} and R^{4b} are each independently H.

7. The compound of claim 1, or pharmaceutically acceptable salt thereof, wherein the compound has the following structure of Formula (XIIa) or (XIIb):

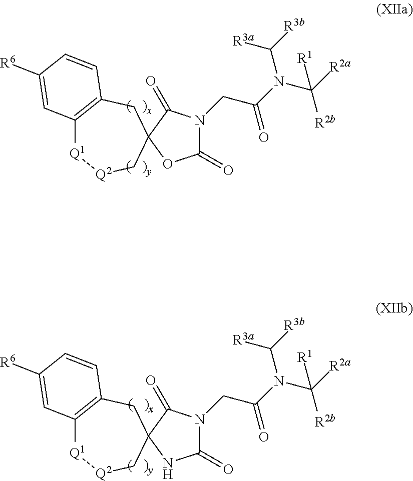

8. The compound of claim 1, or pharmaceutically acceptable salt thereof, wherein the compound has the following structure of Formula (XIIIa) or (XIIIb):

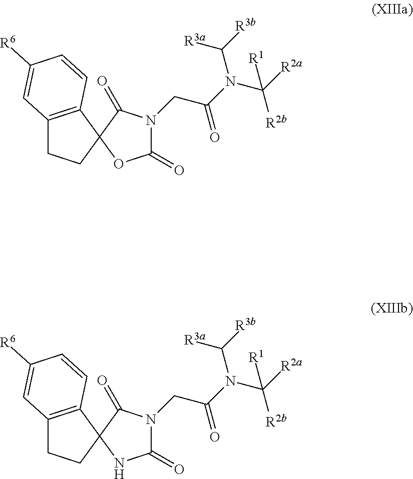

9. The compound of claim 7 or 8, or pharmaceutically acceptable salt thereof, wherein R¹ is carbocyclyl.

10. The compound of claim 7 or 8, or pharmaceutically acceptable salt thereof, wherein R¹⁰ is phenyl, which is unsubstituted.

11. The compound of claim 7 or 8, or pharmaceutically acceptable salt thereof, wherein R¹ is phenyl, which is substituted.

12. The compound of claim 7 or 8, or pharmaceutically acceptable salt thereof, wherein R^{2a} and R^{2b} are each independently H.

13. The compound of claim 7 or 8, or pharmaceutically acceptable salt thereof, wherein R^{2a} is H; and R^{2b} is C₁-C₆ alkyl.

14. The compound of claim 7 or 8, or pharmaceutically acceptable salt thereof, wherein R^{3a} is C₁-C₆ alkyl; and R^{3b} is CF₃.

15. The compound of claim 7 or 8, or pharmaceutically acceptable salt thereof, wherein R^{3a} is C₁-C₆ alkyl; and R^{3b} is cycloalkyl; wherein the R^{3a} C₁-C₆ alkyl is unsubstituted or substituted.

16. The compound of claim 15, or pharmaceutically acceptable salt thereof, wherein R^{3b} is cyclopropyl.

17. The compound of claim 15, or pharmaceutically acceptable salt thereof, wherein R^{3a} is —CH₃.

18. The compound of claim 1 of Formula (IX), or a pharmaceutically acceptable salt or solvate thereof, wherein the compound is selected from the group consisting of
N-benzyl-N-(1-cyclobutylethyl)-2-(2,5-dioxo-2′,3′-dihydrospiro[imidazolidine-4,1′-indene]-1-yl)acetamide;
N-benzyl-N-(1-cyclopropylethyl)-2-(2,5-dioxo-3′,4′-dihydro-2′H-spiro[imidazolidine-4,1′-naphthalene]-1-yl)acetamide;
N-benzyl-N-(1-cyclopropylethyl)-2-(2′,5′-dioxospiro[chroman-4,4′-imidazolidine]-1′-yl)acetamide;
N-benzyl-N-(1-cyclopropylethyl)-2-(2,2′,5-trioxospiro[imidazolidine-4,3′-indoline]-1-yl)acetamide;
N-(4-bromobenzyl)-N-(1-cyclopropylethyl)-2-(2,5-dioxo-2′,3′-dihydrospiro[imidazolidine-4,1′-indene]-1-yl)acetamide;
N-(1-cyclopropylethyl)-2-(2,5-dioxo-2′,3′-dihydrospiro[imidazolidine-4,1′-indene]-1-yl)-N-(furan-2-ylmethyl)acetamide;
N-benzyl-N-(1-cyclopropylethyl)-2-(1′-methyl-2,2′,5-trioxospiro[imidazolidine-4,3′-indoline]-1-yl)acetamide;
N-(1-cyclopropylethyl)-2-(2,5-dioxo-2′,3′-dihydrospiro[imidazolidine-4,1′-indene]-1-yl)-N-(thiophen-2-ylmethyl)acetamide;
N-benzyl-N-(1-cyclopropylethyl)-2-(3-methyl-2,5-dioxo-2′,3′-dihydrospiro[imidazolidine-4,1′-indene]-1-yl)acetamide;
N-(1-cyclopropylethyl)-2-(2,5-dioxo-2′,3′-dihydrospiro[imidazolidine-4,1′-indene]-1-yl)-N-(4-fluorobenzyl)acetamide;
N-(1-cyclopropylethyl)-2-(2,5-dioxo-2′,3′-dihydrospiro[imidazolidine-4,1′-indene]-1-yl)-N-(2-fluorobenzyl)acetamide;
N-(1-cyclopropylethyl)-2-(2,5-dioxo-2′,3′-dihydrospiro[imidazolidine-4,1′-indene]-1-yl)-N-(3-fluorobenzyl)acetamide;
N-benzyl-2-(5′-bromo-2,5-dioxo-2′,3′-dihydrospiro[imidazolidine-4,1′-indene]-1-yl)-N-(1-cyclopropylethyl)acetamide;
2-(5′-bromo-2,5-dioxo-2′,3′-dihydrospiro[imidazolidine-4,1′-indene]-1-yl)-N-(1-cyclopropylethyl)-N-(furan-2-ylmethyl)acetamide;
N-benzyl-N-(1-cyclopropylethyl)-2-(5-fluoro-2,5-dioxo-2′,3′-dihydrospiro[imidazolidine-4,1′-indene]-1-yl)acetamide;
N-benzyl-2-(4′-bromo-2,5-dioxo-2′,3′-dihydrospiro[imidazolidine-4,1′-indene]-1-yl)-N-(1-cyclopropylethyl)acetamide;
N-(1-cyclopropylethyl)-2-(2,5-dioxo-2′,3′-dihydrospiro[imidazolidine-4,1′-indene]-1-yl)-N-(pyridin-3-ylmethyl)acetamide;
N-(1-cyclopropylethyl)-2-(2,5-dioxo-2′,3′-dihydrospiro[imidazolidine-4,1′-indene]-1-yl)-N-(pyridin-2-ylmethyl)acetamide;
N-benzyl-N-(1-cyclopropylethyl)-2-(5-methoxy-2,5-dioxo-2′,3′-dihydrospiro[imidazolidine-4,1′-indene]-1-yl)acetamide;
N-benzyl-N-(1-cyclopropylethyl)-2-(4′-methoxy-2,5-dioxo-2′,3′-dihydrospiro[imidazolidine-4,1′-indene]-1-yl)acetamide;
N-benzyl-N-(1-cyclopropylethyl)-2-(2′,5′-dioxo-6,7-dihydrospiro[cyclopenta[b]pyridine-5,4′-imidazolidine]-1′-yl)acetamide;
N-benzyl-2-(5′-cyano-2,5-dioxo-2′,3′-dihydrospiro[imidazolidine-4,1′-indene]-1-yl)-N-(1-cyclopropylethyl)acetamide;
N-(1-cyclopropylethyl)-2-(2,5-dioxo-2′,3′-dihydrospiro[imidazolidine-4,1′-indene]-1-yl)-N-(pyridin-4-ylmethyl)acetamide;
N-benzyl-2-(4′-chloro-2,5-dioxo-2′,3′-dihydrospiro[imidazolidine-4,1′-indene]-1-yl)-N-(1-cyclopropropylethyl)acetamide;
N-benzyl-2-(2,5-dioxo-2′,3′-dihydrospiro[imidazolidine-4,1′-indene]-1-yl)-N-isopropylacetamide;
N-benzyl-N-sec-butyl-2-(2,5-dioxo-2′,3′-dihydrospiro[imidazolidine-4,1′-indene]-1-yl)acetamide;
N-(1-cyclobutylethyl)-2-(2,5-dioxo-2′,3′-dihydrospiro[imidazolidine-4,1′-indene]-1-yl)-N-(4-fluorobenzyl)acetamide
N-benzyl-2-(4′-bromo-2,5-dioxo-2′,3′-dihydrospiro[imidazolidine-4,1′-indene]-1-yl)-N—((R)-1-cyclobutylethyl)acetamide;
2-(4′-bromo-2,5-dioxo-2′,3′-dihydrospiro[imidazolidine-4,1′-indene]-1-yl)-N-(1-cyclopropylethyl)-N-(4-fluorobenzyl)acetamide;
N-benzyl-2-(5′-bromo-2,5-dioxo-2′,3′-dihydrospiro[imidazolidine-4,1′-indene]-1-yl)-N-(1-cyclobutylethyl)acetamide;
2-(5′-bromo-2,5-dioxo-2′,3′-dihydrospiro[imidazolidine-4,1′-indene]-1-yl)-N-(1-cyclopropylethyl)-N-(4-fluorobenzyl)acetamide;
1-(2-(benzyl(1-cyclopropylethyl)amino)-2-oxoethyl)-2,5-dioxo-2′,3′-dihydrospiro[imidazolidine-4,1′-indene]-5′-carboxamide;
N-benzyl-2-(4′-cyano-2,5-dioxo-2′,3′-dihydrospiro[imidazolidine-4,1′-indene]-1-yl)-N-(1-cyclopropropylethyl)acetamide
1-(2-(benzyl(1-cyclopropylethyl)amino)-2-oxoethyl)-2,5-dioxo-2′,3′-dihydrospiro[imidazolidine-4,1′-indene]-4′-carboxamide;
N-(1-cyclopropylethyl)-2-(2,5-dioxo-2′,3′-dihydrospiro[imidazolidine-4,1′-indene]-1-yl)-N-(naphthalen-2-ylmethyl)acetamide;
N-((5-bromofuran-2-yl)methyl)-N-(1-cyclopropylethyl)-2-(2,5-dioxo-2′,3′-dihydrospiro[imidazolidine-4,1′-indene]-1-yl)acetamide;
N-benzyl-N—((S)-1-cyclobutylethyl)-2-((S)-2,5-dioxo-2′,3′-dihydrospiro[imidazolidine-4,1′-indene]-1-yl)acetamide;
N-benzyl-N—((R)-1-cyclobutylethyl)-2-((S)-2,5-dioxo-2′,3′-dihydrospiro[imidazolidine-4,1′-indene]-1-yl)acetamide;
N-benzyl-N-(1-cyclopropylethyl)-2-(2′,5′-dioxo-5,6-dihydrospiro[cyclopenta[b]thiophene-4,4′-imidazolidine]-1′-yl)acetamide;
N-benzyl-N-(1-cyclopropylethyl)-2-(2′,5′-dioxo-4,5-dihydrospiro[cyclopenta[b]thiophene-6,4′-imidazolidine]-1′-yl)acetamide;
N-((1H-indol-5-yl)methyl)-N-(1-cyclopropylethyl)-2-(2,5-dioxo-2′ 3′-dihydrospiro[imidazolidine-4,1′-indene]-1-yl)acetamide;
N-benzyl-N-(1-cyclopropylethyl)-2-(2′,4′-dioxo-2,3-dihydrospiro[indene-1,5′-ozazolidine]-3′-yl)acetamide;
N-benzyl-2-(2,5-dioxo-2′,3′-dihydrospiro[imidazolidine-4,1′-indene]-1-yl)-N-(pentan-3-yl)acetamide;
N-benzyl-N-(1-cyclopropylethyl)-2-(3′-methoxy-2,5-dioxo-2′,3′-dihydrospiro[imidazolidine-4,1′-indene]-1-yl)acetamide;
N-benzyl-N-(1-cyclopropylethyl)-2-(5′-(methylthio)-2,5-dioxo-2′,3′-dihydrospiro[imidazolidine-4,1′-indene]-1-yl)acetamide;
N-benzyl-2-(7-chloro-2,5-dioxo-2′,3′-dihydrospiro[imidazolidine-4,1′-indene]-1-yl)-N-(1-cyclobutylethyl)acetamide;
N-benzyl-N-(1-cyclopropylethyl)-2-(5′-(hydroxymethyl)-2,5-dioxo-2′,3′-dihydrospiro[imidazolidine-4,1′-indene]-1-yl)acetamide;
N-benzyl-N-(1-cyclopropylethyl)-2-(5′-((difluoromethoxy)methyl)-2,5-dioxo-2′,3′-dihydrospiro[imidazolidine-4,1′-indene]-1-yl)acetamide;
N-benzyl-2-(5′-cyclopropyl-2,5-dioxo-2′,3′-dihydrospiro[imidazolidine-4,1′-indene]-1-yl)-N-(1-cyclopropylethyl)acetamide;
N-benzyl-N-(1-cyclobutylethyl)-2-(3′-methyl-2,5-dioxo-2′,3′-dihydrospiro[imidazolidine-4,1′-indene]-1-yl)acetamide;
N-benzyl-N-(1-cyclobutylethyl)-2-(5′-(difluoromethoxy)-2,5-dioxo-2′,3′-dihydrospiro[imidazolidine-4,1′-indene]1-yl)acetamide;
N-benzyl-N-(1-cyclopropylethyl)-2-(4′-methyl-2,5-dioxo-2′,3′-dihydrospiro[imidazolidine-4,1′-indene]-1-yl)acetamide;
2-(5′-acetamido-2,5-dioxo-2′,3′-dihydrospiro[imidazolidine-4,1′-indene]-1-yl)-N-benzyl-N-(1-cyclopropylethyl)acetamide;
N-benzyl-N-(1-cyclopropylethyl)-2-(5′-methyl-2,5-dioxo-2′,3′-dihydrospiro[imidazolidine-4,1′-indene]-1-yl)acetamide;
1-(2-(benzyl((S)-1-cyclopropylethyl)amino)-2-oxoethyl)-N-(cyanomethyl)-2,5-dioxo-2′,3′-dihydrospiro[imidazolidine-4,1′-indene]-5′-carboxamide;
N-benzyl-N-(1-cyclopropylethyl)-2-(5′-ethynyl-2,5-dioxo-2′,3′-dihydrospiro[imidazolidine-4,1′-indene]-1-yl)acetamide;
N-benzyl-N-(1-cyclopropylethyl)-2-(5′-(3-hydroxyprop-1-ynyl)-2,5-dioxo-2′,3′-dihydrospiro[imidazolidine-4,1′-indene]-1-yl)acetamide;
N-benzyl-N-(1-cyclobutylethyl)-2-(6′-methoxy-2,5-dioxo-2′,3′-dihydrospiro[imidazolidine-4,1′-indene]-1-yl)acetamide;
N-benzyl-N-(1-cyclobutylethyl)-2-(2-imino-5-oxo-2′,3′-dihydrospiro[imidazolidine-4,1′-indene]-1-yl)acetamide;
N-benzyl-N-(dicyclopropylmethyl)-2-(2,5-dioxo-2′,3′-dihydrospiro[imidazolidine-4,1′-indene]-1-yl)acetamide;
N-benzyl-2-(6-chloro-2,5-dioxo-2,3′-dihydrospiro[imidazolidine-4,1′-indene]-1-yl)-N-(1-cyclobutylethyl)acetamide;
N-benzyl-N-(1-cyclopropylethyl)-2-(2,5-dioxo-5′-(1H-pyrazol-5-yl)-2′,3′-dihydrospiro[imidazolidine-4,1′-indene]-1-yl)acetamide;
N-(4-chlorobenzyl)-N—((S)-1-cyclobutylethyl)-2-((S)-2,5-dioxo-2′,3′-dihydrospiro[imidazolidine-4,1′-indene]-1-yl)acetamide;
N-benzyl-N-(1-cyclopropylethyl)-2-(2,5-dioxo-5′-(1H-1,2,3-triazol-4-yl)-2′,3′-dihydrospiro[imidazolidine-4,1′-indene]-1-yl)acetamide;
N-benzyl-N—((S)-1-cyclobutylethyl)-2-((3′R)-3′-hydroxy-2,5-dioxo-2′,3′-dihydrospiro[imidazolidine-4,1′-indene]-1-yl)acetamide;
N-benzyl-N—((S)-1-cyclobutylethyl)-2-((3′S)-3′-hydroxy-2,5-dioxo-2′,3′-dihydrospiro[imidazolidine-4,1′-indene]-1-yl)acetamide;
N-(4-bromobenzyl)-N—((S)-1-cyclobutylethyl)-2-((S)-2,5-dioxo-2′,3′-dihydrospiro[imidazolidine-4,1′-indene]-1-yl)acetamide;
N-benzyl-N-(1-cyclopropylethyl)-2-(2,2′,5′-trioxo-1,2,6,7-tetrahydrospiro[cyclopenta[b]pyridine-5,4′-imidazolidine]-1′-yl)acetamide;
2-(5′-(1H-imidazol-2-yl)-2,5-dioxo-2′,3′-dihydrospiro[imidazolidine-4,1′-indene]-1-yl)-N-benzyl-N-(1-cyclopropylethyl)acetamide;
N-benzyl-2-(5-bromo-2′,4′-dioxo-2,3-dihydrospiro[indene-1,5′-ozazolidine]-3′-yl)-N-(1-cyclopropylethyl)acetamide;
N-benzyl-2-(2′,4′-dioxo-2,3-dihydrospiro[indene-1,5′-ozazolidine]-3′-yl)-N-(pentan-3-yl)acetamide;
N-benzyl-N-(1-cyclobutylethyl)-2-(2′,4′-dioxo-2,3-dihydrospiro[indene-1,5′-ozazolidine]-3′-yl)acetamide;
N-(1-cyclopropylethyl)-2-(2′,4-dioxo-2,3-dihydrospiro[indene-1,5′-ozazolidine]-3′-yl)-N-(4-fluorobenzyl)acetamide;
N-benzyl-N-(1-cyclopropylethyl)-2-(4-methoxy-2′,4′-dioxo-2,3-dihydrospiro[indene-1,5′-ozazolidine]-3′-yl)acetamide;
N—((S)-1-cyclobutylethyl)-2-((S)-2,5-dioxo-2′,3′-dihydrospiro[imidazolidine-4,1′-indene]-1-yl)-N-(4-(methylthio)benzyl)acetamide;
N-benzyl-N-(1-cyclopropylethyl)-2-(2,5-dioxo-5′-(1H-1,2,3-triazol-1-yl)-2′,3′-dihydrospiro[imidazolidine-4,1′-indene]-1-yl)acetamide;
N-benzyl-N-(1-cyclopropylethyl)-2-(2,5-dioxo-5′-(2H-1,2,3-triazol-2-yl)-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(4-fluorobenzyl)acetamide;
N-(4-cyanobenzyl)-N—((S)-1-cyclobutylethyl)-2-((S)-2,5-dioxo-2′,3′-dihydrospiro[imidazolidine-4,1′-indene]-1-yl)acetamide;
4-((N—((S)-1-cyclobutylethyl)-2-((S)-2,5-dioxo-2′,3′-dihydrospiro[imidazolidine-4,1′-indene]-1-yl)acetamido)methyl)benzamide;
N-benzyl-N-(1-cyclopropylethyl)-2-(5′-(methoxymethyl)-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(4-methylbenzyl)acetamide;
N—((S)-1-cyclobutylethyl)-2-((S)-2,5-dioxo-2′,3′-dihydrospiro[imidazolidine-4,1′-indene]-1-yl)-N-(4-methoxybenzyl)acetamide;
N—((S)-1-cyclobutyl ethyl)-2-((S)-2,5-dioxo-2′,3′-dihydrospiro[imidazolidine-4,1′-indene]-1-yl)-N-(3-methylbenzyl)acetamide;
N—((S)-1-cyclobutylethyl)-2-((S)-2,5-dioxo-2,35-dihydrospiro[imidazolidine-4,1′-indene]-1-yl)-N-(2-methylbenzyl)acetamide;
N-((1H-1,2,3-triazol-4-yl)methyl)-N—((S)-1-cyclobutylethyl)-2-((S)-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4, 1-indene]-1-yl)-N-(4-(methylsulfonyl)benzyl)acetamide;
2-(5-bromo-2′,4′-dioxo-2,3-dihydrospiro[indene-1,5-ozazolidine]-3′-yl)-N-(1-cyclopropylethyl)-N-(4-fluorobenzyl)acetamide;
N-benzyl-2-(5-cyano-2′,4′-dioxo-2,3-dihydrospiro[indene-1,5-ozazolidine]-3′-yl)-N-(1-cyclopropropylethyl)acetamide;
N-benzyl-N—((S)-1-cyclopropylethyl)-2-(2,3′,5-trioxo-2′,3′-dihydrospiro[imidazolidine-4,1′-indene]-1-yl)acetamide;
N-benzyl-2-(5′-(2-cyanoacetamido)-2,5-dioxo-2′,3′-dihydrospiro[imidazolidine-4,1′-indene]-1-yl)-N—((S)-1-cyclopropylethyl)acetamide;
methyl 1-(2-(benzyl((S)-1-cyclopropyl ethyl)amino)-2-oxoethyl)-2,5-dioxo-2′,3′-dihydrospiro[imidazolidine-4,1′-indene]-5′-ylcarbamate;
N-benzyl-N—((S)-1-cyclopropylethyl)-2-(7′-fluoro-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(1-phenylethyl)acetamide;
N-benzyl-2-(7′-cyano-2,5-dioxo-2′,3′-dihydrospiro[imidazolidine-4,1′-indene]-1-yl)-N—((S)-1-cyclopropylethyl)acetamide;
(S)-2-(5-acetamido-2′,4′-dioxo-2,3-dihydrospiro[indene-1,5′-ozazolidine]-3′-yl)-N-benzyl-N-(1-cyclopropylethyl)acetamide;
3′-(2-(benzyl(1-cyclopropylethyl)amino)-2-oxoethyl)-2′,4′-dioxo-2,3-dihydrospiro[indene-1,5′-ozazolidine]-5-carboxamide;
N-benzyl-N—((S)-1-cyclopropylethyl)-2-(5′-(3-methylureido)-2,5-dioxo-2′,3′-dihydrospiro[imidazolidine-4,1′-indene]-1-yl)acetamide;
N-benzyl-N—((S)-1-cyclopropylethyl)-2-(5′-(3-methyl-3-(methylcarbamoyl)ureido)-2,5-dioxo-2′,3′-dihydrospiro[imidazolidine-4,1′-indene]-1-yl)acetamide;
N-benzyl-N-(1-cyclopropylethyl)-2-(5-methoxy-2′,4′-dioxo-2,3-dihydrospiro[indene-1,5′-ozazolidine]-3′-yl)acetamide;
N—((S)-1-cyclopropylethyl)-2-((S)-2,5-dioxo-2′,3′-dihydrospiro[imidazolidine-4,1′-indene]-1-yl)-N-(4-ethynylbenzyl)acetamide;
N—((S)-1-cyclobutylethyl)-2-((S)-2,5-dioxo-2′,3′-dihydrospiro[imidazolidine-4,1′-indene]-1-yl)-N-((1-methyl-1H-1,2,3-triazol-4-yl)methyl)acetamide;
N-((1-(cyanomethyl)-1H-1,2,3-triazol-4-yl)methyl)-N—((S)-1-cyclobutylethyl)-2-((S)-2,5-dioxo-2′,3′-dihydrospiro[imidazolidine-4,1′-indene]-1-yl)acetamide;
N—((S)-1-cyclobutylethyl)-2-((S)-2,5-dioxo-2′,3′-dihydrospiro[imidazolidine-4,1′-indene]-1-yl)-N-(4-ureidobenzyl)acetamide;
N-benzyl-N—((S)-1-cyclopropylethyl)-2-(5-(3-hydroxyprop-1-ynyl)-2′,4′-dioxo-2,3-dihydrospiro[indene-1,5′-ozazolidine]-3′-yl)acetamide;
N-benzyl-N—((S)-1-cyclopropylethyl)-2-(5′-(3-hydroxy-3-methylbut-1-ynyl)-2,5-dioxo-2′,3′-dihydrospiro[imidazolidine-4,1′-indene]-1-yl)acetamide;
N-((1-(2-amino-2-oxoethyl)-1H-1,2,3-triazol-4-yl)methyl)-N—((S)-1-cyclobutylethyl)-2-((S)-2,5-dioxo-2′,3′-dihydrospiro[imidazolidine-4,1′-indene]-1-yl)acetamide;
(S)—N-benzyl-N-(1-cyclopropylethyl)-2-(2,5-dioxo-5′-(6-oxo-1,6-dihydropyridin-3-yl)-2′,3′-dihydrospiro[imidazolidine-4,1′-indene]-1-yl)acetamide;
N-benzyl-N—((S)-1-cyclopropylethyl)-2-((S)-2,5-dioxo-5′-(1H-pyrazol-3-yl)-2′,3′-dihydrospiro[imidazolidine-4,1′-indene]-1-yl)acetamide;
N-benzyl-N—((S)-1-cyclopropylethyl)-2-(5-(methylsulfonamido)-2′,4′-dioxo-2′,3′-dihydrospiro[indene-1,5′-ozazolidine]-3′-yl)acetamide;
N-benzyl-N—((S)-1-cyclopropylethyl)-2-(5′-(methylsulfonamido)-2,5-dioxo-2′,3′-dihydrospiro[imidazolidine-4,1′-indene]-1-yl)acetamide;
N-benzyl-2-(5′-(2-cyanoacetamido)-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(2,5-dioxo-5′-(2H-tetrazol-5-yl)-2′,3′-dihydrospiro[imidazolidine-4,1′-indene]-1-yl)acetamide;
(S)—N-benzyl-N-(1-cyclopropylethyl)-2-(3′-(hydroxyimino)-2,5-dioxo-2′,3′-dihydrospiro[imidazolidine-4,1′-indene]-1-yl)acetamide;
(S)—N-benzyl-N-(1-cyclopropylethyl)-2-(3′-(methoxyimino)-2,5-dioxo-2′,3′-dihydrospiro[imidazolidine-4,1′-indene]-1-yl)acetamide;
(S)-3′-(2-(benzyl(1-cyclopropylethyl)amino)-2-oxoethyl)-2′,4′-dioxo-2,3-dihydrospiro[indene-1,5′-ozazolidine]-5-carboxamide;
N-benzyl-N—((S)-1-cyclopropylethyl)-2-((S)-2,5-dioxo-5′-(6-oxo-1,6-dihydropyridin-3-yl)-2′,3′-dihydrospiro[imidazolidine-4,1′-indene]-1-yl)acetamide;
N-benzyl-N—((S)-1-cyclopropylethyl)-2-((S)-5′-(3-hydroxyprop-1-ynyl)-2,5-dioxo-2,3′-dihydrospiro[imidazolidine-4,1′-indene]-1-yl)acetamide;
N-benzyl-N—((S)-1-cyclopropylethyl)-2-((S)-2,5-dioxo-5′-(2H-1,2,3-triazol-4-yl)-2′,3′-dihydrospiro[imidazolidine-4,1′-indene]-1-yl)acetamide;
N-benzyl-N—((S)-1-cyclopropylethyl)-2-(5-(morpholinomethyl)-2′,4′-dioxo-2,3-dihydrospiro[indene-1,5′-ozazolidine]-3′-yl)acetamide;
N-benzyl-N—((S)-1-cyclopropylethyl)-2-(5-(3-methylureido)-2′,4′-dioxo-2, 3-dihydrospiro[indene-1,5′-ozazolidine]-3′-yl)acetamide;
N-benzyl-N—((S)-1-cyclopropylethyl)-2-(5′-(3-hydroxypyrrolidin-1-yl)-2,5-dioxo-2′,3′-dihydrospiro[imidazolidine-4,1′-indene]-1-yl)acetamide;
N-benzyl-N—((S)-1-cyclopropylethyl)-2-((S)-5′-(3-hydroxy-3-methylbut-1-ynyl)-2,5-dioxo-2′,3′-dihydrospiro[imidazolidine-4,1′-indene]-1-yl)acetamide;
(S)—N-(3′-(2-(benzyl(1-cyclopropylethyl)amino)-2-oxoethyl)-2′,4′-dioxo-2,3-dihydrospiro[indene-1,5′-ozazolidine]-5-yl)-3-oxobutanamide;
N-benzyl-N—((S)-1-cyclopropylethyl)-2-(5-morpholino-2′,4′-dioxo-2,3-dihydrospiro[indene-1,5′-ozazolidine]-3′-yl)acetamide;
N-benzyl-2-(5-(2-cyanoacetamido)-2′,4′-dioxo-2,3-dihydrospiro[indene-1,5′-ozazolidine]-3′-yl)-N—((S)-1-cyclopropylethyl)acetamide;
N-benzyl-2-((S)-5′-cyano-2,5-dioxo-2′,3′-dihydrospiro[imidazolidine-4,1′-indene]-1-yl)-N—((S)-1-cyclopropylethyl)acetamide;
1-(2-(benzyl((S)-1-cyclopropylethyl)amino)-2-oxoethyl)-2,5-dioxo-2′,3′-dihydrospiro[imidazolidine-4,1′-indene]-3′-carboxylic acid;
N-benzyl-N—((S)-1-cyclopropylethyl)-2-((S)-5′-(3-methylureido)-2,5-dioxo-2′,3′-dihydrospiro[imidazolidine-4,1′-indene]-1-yl)acetamide;
N-benzyl-N—((S)-1-cyclopropylethyl)-2-(2′,4′-dioxo-5-(2-oxa-6-azaspiro[3.3]heptan-6-yl)-2,3-dihydrospiro[indene-1,5′-ozazolidine]-3′-yl)acetamide;
N-benzyl-N—((S)-1-cyclopropylethyl)-2-(2′,4′-dioxo-5-(piperidin-1-ylmethyl)-2,3-dihydrospiro[indene-1,5′-ozazolidine]-3′-yl)acetamide;
N-benzyl-N—((S)-1-cyclopropylethyl)-2-(2′,4′-dioxo-5-(piperazin-1-yl)methyl)-2,3-dihydrospiro[indene-1,5′-ozazolidine]-3′-yl)acetamide;
N-benzyl-N—((S)-1-cyclopropylethyl)-2-(5-((4-hydroxypiperidin-1-yl)methyl)-2′,4′-dioxo-2,3-dihydrospiro[indene-1,5′-ozazolidine]-3′-yl)acetamide;
(S)-2-amino-N-(3′-(2-(benzyl(1-cyclopropylethyl)amino)-2-oxoethyl)-2′,4′-dioxo-2,3-dihydrospiro[indene-1,5′-ozazolidine]-5-yl)-2-methylpropanamide;
(S)—N-benzyl-N-(1-cyclopropylethyl)-2-(2′,4′-dioxo-5-(piperazin-1-yl)-2,3-dihydrospiro[indene-1,5′-ozazolidine]-3′-yl)acetamide;
N-benzyl-N—((S)-1-cyclopropylethyl)-2-(5′-(3-hydroxyazetidin-1-yl)-2,5-dioxo-2,3′-dihydrospiro[imidazolidine-4,1′-indene]-1-yl)acetamide;
N-benzyl-2-((S)-5′-(2-cyanoacetamido)-2,5-dioxo-2′,3′-dihydrospiro[imidazolidine-4,1′-indene]-1-yl)-N—((S)-1-cyclopropylethyl)acetamide;
1-(2-(benzyl((S)-1-cyclopropylethyl)amino)-2-oxoethyl)-2,5-dioxo-2′,3′-dihydrospiro[imidazolidine-4,1′-indene]-3′-carboxamide;
N-benzyl-2-(3′-cyano-2,5-dioxo-2′,3′-dihydrospiro[imidazolidine-4,1′-indene]-1-yl)-N—((S)-1-cyclopropylethyl)acetamide;
N-benzyl-N—((S)-1-cyclopropylethyl)-2-(5′-(2,4-dioxo-3,4-dihydropyrimidin-1(2H)-yl)-2,5-dioxo-2′,3′-dihydrospiro[imidazolidine-4,1′-indene]-1-yl)acetamide;
N-benzyl-N—((S)-1-cyclopropylethyl)-2-(5-hydroxy-2′,4′-dioxo-2,3-dihydrospiro[indene-1,5′-ozazolidine]-3′-yl)acetamide;
methyl 2-(3′-(2-(benzyl((S)-1-cyclopropylethyl)amino)-2-oxoethyl)-2′,4′-dioxo-2,3-dihydrospiro[indene-1,5′-ozazolidine]-5-yloxy)acetate;
N-benzyl-N—((S)-1-cyclopropylethyl)-2-(2′,4′-dioxo-5-(prop-2-ynyloxy)-2,3-dihydrospiro[indene-1,5′-ozazolidine]-3′-yl)acetamide;
N-(1-(2-(benzyl((S)-1-cyclopropylethyl)amino)-2-oxoethyl)-2,5-dioxo-2′,3′-dihydrospiro[imidazolidine-4,1′-indene]-5′-yl)acrylamide;
N-benzyl-2-(5′-(2-cyano-1-hydroxyallyl)-2,5-dioxo-2′,3′-dihydrospiro[imidazolidine-4,1′-indene]-1-yl)-N—((S)-1-cyclopropylethyl)acetamide;
(S)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)-3-methylbutanamide;
(2S)-2-amino-N-(3′-(2-(benzyl((S)-1-cyclopropylethyl)amino)-2-oxoethyl)-2′,4′-dioxo-2,3 dihydrospiro[indene-1,5′-ozazolidine]-5-yl)propanamide;
N-benzyl-N—((S)-1-cyclopropylethyl)-2-(5′-(2,4-dioxo-1,2,3,4-tetrahydropyrimidin-5-yl)-2,5-dioxo-2′,3′-dihydrospiro[imidazolidine-4,1′-indene]-1-yl)acetamide;
1-(2-(benzyl((S)-1-cyclopropylethyl)amino)-2-oxoethyl)-2,5-dioxo-2′,3′-dihydrospiro[imidazolidine-4,1′-indene]-5′-carboxylic acid;
2-(3′-(2-(benzyl((S)-1-cyclopropylethyl)amino)-2-oxoethyl)-2′,4′-dioxo-2,3-dihydrospiro[indene-1,5′-ozazolidine]-5-ylamino)acetic acid;
N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-cyano-3-oxobutanamide;
(S)—N-(1-(2-(benzyl(1-cyclopropylethyl)amino)-2-oxoethyl)-2,5-dioxo-2′,3′-dihydrospiro[imidazolidine-4,1′-indene]-5′-yl)-3-oxobutanamide;
N-(1-(2-(benzyl((S)-1-cyclopropylethyl)amino)-2-oxoethyl)-2,5-dioxo-2′,3′-dihydrospiro[imidazolidine-4,1′-indene]-5′-yl)-2-cyano-3-oxobutanamide;
2-(3′-(2-(benzyl((S)-1-cyclopropylethyl)amino)-2-oxoethyl)-2′,4′-dioxo-2,3-dihydrospiro[indene-1,5′-ozazolidine]-5-yloxy)acetic acid;
N-benzyl-N—((S)-1-cyclopropylethyl)-2-(5-(oxetan-3-ylamino)-2′,4′-dioxo-2,3-dihydrospiro[indene-1,5′-ozazolidine]-3′-yl)acetamide;
N-benzyl-N—((S)-1-cyclopropylethyl)-2-(2′,4-dioxo-5-(tetrahydro-2H-pyran-4-ylamino)-2,3-dihydrospiro[indene-1,5′-ozazolidine]-3′-yl)acetamide;
N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-hydroxypropanamide;
(E)-N-(1-(2-(benzyl((S)-1-cyclopropylethyl)amino)-2-oxoethyl)-2,5-dioxo-2,3′-dihydrospiro[imidazolidine-4,1′-indene]-5′-yl)-4-(piperidin-1-yl)but-2-enamide;
N-benzyl-2-((S)-2,5-dioxo-dihydrospiro[imidazolidine-4,1′-indene]-1-yl)-N-(1-(oxetan-3-yl)ethyl)acetamide;
1-(3′-(2-(benzyl((S)-1-cyclopropylethyl)amino)-2-oxoethyl)-2′,4′-dioxo-2,3-dihydrospiro[indene-1,5′-ozazolidine]-5-yl)azetidine-3-carboxamide;
2-amino-N-(3′-(2-(benzyl(dicyclopropylethyl)amino)-2-oxoethyl)-2′,4′-dioxo-2,3-dihydrospiro[indene-1,5′-ozazolidine]-5-yl)-2-methylpropanamide;
N-benzyl-N-(dicyclopropylmethyl)-2-(5-(3-methylureido)-2′,4′-dioxo-2,3-dihydrospiro[indene-1,5′-ozazolidine]-3′-yl)acetamide;
N-benzyl-N—((S)-1-cyclopropylethyl)-2-(5-(2-(dimethylamino)ethoxy)-2′,4′-dioxo-2,3-dihydrospiro[indene-1,5′-ozazolidine]-3′-yl)acetamide;
2-(5-((1H-1,2,3-triazol-4-yl)methoxy)-2′,4′-dioxo-2,3-dihydrospiro[indene-1,5′-ozazolidine]-3′-yl)-N-benzyl-N—((S)-1-cyclopropylethyl)acetamide;
N-benzyl-N—((S)-1-cyclopropylethyl)-2-(2′,4′-dioxo-5-(6-oxo-1,6-dihydropyridin-3-yl)-2,3-dihydrospiro[indene-1,5′-ozazolidine]-3′-yl)acetamide;
N-benzyl-N—((S)-1-cyclopropylethyl)-2-(5-(2-hydroxyethoxy)-2′,4′-dioxo-2,3-dihydrospiro[indene-1,5′-ozazolidine]-3′-yl)acetamide;
3′-(2-(benzyl((S)-1-cyclopropylethyl)amino)-2-oxoethyl)-2′,4′-dioxo-2,3-dihydrospiro[indene-1,5′-ozazolidine]-5-ylboronic acid;
N-benzyl-N—((S)-1-cyclopropylethyl)-2-(5-(3-(hydroxymethyl)azetidin-1-yl)-2′,4′-dioxo-2,3-dihydrospiro[indene-1,5′-ozazolidine]-3′-yl)acetamide;
2-amino-N-((1′S)-1-((4S)-3-benzyl-4-cyclopropyl-2-oxopentyl)-2,5-dioxo-2′,3′-dihydrospiro[imidazolidine-4,1′-indene]-5′-yl)-2-methylpropanamide;
N—((S)-1-cyclopropylethyl)-2-(2′,4′-dioxo-5-(6-oxo-1,6-dihydropyridin-3-yl)-2,3-dihydrospiro[indene-1,5′-ozazolidine]-3′-yl)-N-(4-fluorobenzyl)acetamide;
(S)-2-amino-N—((S)-1-(2-(benzyl((S)-1-cyclopropylethyl)amino)-2-oxoethyl)-2,5-dioxo-2′,3′-dihydrospiro[imidazolidine-4,1′-indene]-5′-yl)propanamide;
1-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)cyclobutanecarboxamide;
N-benzyl-N—((S)-1-cyclopropylethyl)-2-(5′-(2-hydroxy-5-oxo-2,5-dihydrofuran-2-yl)-2,5-dioxo-2′,3′-dihydrospiro[imidazolidine-4,1′-indene]-1-yl)acetamide;
2-(5-(2-cyanoacetamido)-2′,4′-dioxo-2,3-dihydrospiro[indene-1,5-ozazolidine]-3′-yl)-N—((S)-1-cyclopropylethyl)-N-(4-fluorobenzyl)acetamide;
N—((S)-1-cyclopropylethyl)-N-(4-fluorobenzyl)-2-(5-(3-methylureido)-2′,4′-dioxo-2,3-dihydrospiro[indene-1,5′-ozazolidine]-3′-yl)acetamide;
5′-(azetidin-3-ylmethylamino)-1-((4S)-3-benzyl-4-cyclopropyl-2-oxopentyl)-2′,3′-dihydrospiro[imidazolidine-4,1′-indene]-2,5-dione;
3′-((4S)-3-benzyl-4-cyclopropyl-2-oxopentyl)-5-(1H-pyrazol-5-yl)-2,3-dihydrospiro[indene-1,5′-ozazolidine]-2′,4′-dione;
2-amino-N-(3-(2-(benzyl((S)-1-cyclopropylethyl)amino)-2-oxoethyl)-4′-oxo-2,3-dihydrospiro[indene-1,5′-ozazolidine]-5-yl)-3-hydroxypropanamide;
(S)—N-(3′-(2-(benzyl((S)-1-cyclopropylethyl)amino)-2-oxoethyl)-2′,4′-dioxo-2,3-dihydrospiro[indene-1,5′-ozazolidine]-5-yl)-2-hydroxy-2-methylpropanamide;
1-amino-N-(3′-(2-(benzyl((S)-1-cyclopropylethyl)amino)-2-oxoethyl)-2′,4′-dioxo-2′,3′-dihydrospiro[indene-1,5′-ozazolidine]-5-yl)cyclopropanecarboxamide;
2-amino-N-(3′-(2-(((S)-1-cyclopropylethyl)(4-fluorobenzyl)amino)-2-oxoethyl)-2′,4′-dioxo-2,3-dihydrospiro[indene-1,5′-ozazolidine]-5-yl)-2-methylpropanamide;
(S)-2-(5-(azetidin-3-ylamino)-2′,4′-dioxo-2,3-dihydrospiro[indene-1,5′-ozazolidine]-3′-yl)-N-benzyl-N-(1-cyclopropylethyl)acetamide;
1-amino-N—((S)-1-(2-(benzyl((S)-1-cyclopropylethyl)amino)-2-oxoethyl)-2,5-dioxo-2′,3′-dihydrospiro[imidazolidine-4,1′-indene]-5′-yl)cyclobutanecarboxamide;
(2R)-2-amino-N-(3′-(2-(benzyl((S)-1-cyclopropylethyl)amino)-2-oxoethyl)-2′,4′-dioxo-2,3-dihydrospiro[indene-1,5′-ozazolidine]-5-yl)propanamide;
N-benzyl-N—((S)-1-cyclopropylethyl)-2-(2,5-dioxo-5′-(thiazol-2-ylamino)-2′,3′-dihydrospiro[imidazolidine-4,1′-indene]-1-yl)acetamide;
(S)-2-(5-(azetidin-3-ylamino)-2,5-dioxo-2′,3′-dihydrospiro[imidazolidine-4,1′-indene]-1-yl)-N-benzyl-N-(1-cyclopropylethyl)acetamide;
(S)-2-(3′-(2-(benzyl(I-cyclopropylethyl)amino)-2-oxoethyl)-2′,4′-dioxo-2,3-dihydrospiro[indene-1,5′-ozazolidine]-5-ylcarboxamido)acetic acid;
2-((S)-5-acetamido-2′,4′-dioxo-2,3-dihydrospiro[indene-1,5′-ozazolidine]-3′-yl)-N-benzyl-N—((S)-1-cyclopropylethyl)acetamide;
2-((R)-5-acetamido-2′,4′-dioxo-2,3-dihydrospiro[indene-1,5′-ozazolidine]-3′-yl)-N-benzyl-N—((S)-1-cyclopropylethyl)acetamide;
N-benzyl-N-(1-cyclopropylethyl)-2-(2,5-dioxo-1′,3′-dihydrospiro[imidazolidine-4, 2′-indene]-1-yl)acetamide;
N-benzyl-N-(1-cyclopropylethyl)-2-(8′-methoxy-2,5-dioxo-3′,4′-dihydro-1′H-spiro[imidazolidine-4,2′-naphthalene]-1-yl)acetamide;
N-benzyl-N-(1-cyclobutylethyl)-2-(3′,3′-dimethyl-2,5-dioxo-2,3′-dihydrospiro[imidazolidine-4,1′-indene]-1-yl)acetamide;
N-benzyl-2-[5′-(5-cyano-6-cyano-6-oxo-1,6-dihydropyridin-3-yl)-2,5-dioxo-2′,3′-dihydrospiro[imidazolidine-4,1′-indene]-1-yl]-N-(1-cyclopropylethyl)acetamide;
N-benzyl-2-{5-[(methylcarbamoyl)amino]-3′,5′-dioxo-2,3-dihydrospiro[indene-1,2′-[1,4]ozazolidine]-4′-yl}-N-(pentan-3-yl)acetamide;
2-amino-N-(4′-{[benzyl(pentat-3-yl)carbamoyl]methyl}-3′,5′-dioxo-2,3-dihydrospiro[indene-1,2′-[1,4]ozazolidine]-5-yl)-2-methylpropanamide;
(2R)-2-amino-N-[(4S)-1-({benzyl[(1S)-1-cyclopropylethyl]carbamoyl}methyl)-2,5-dioxo-2′,3′-dihydrospiro[imidazolidine-4,1′-indene]-5′-yl]propanamide;
N-benzyl-N-[(1S)-1-cyclopropylethyl]-2-{5′-[(1H-imidazol-2-yl)amino]-2,5-dioxo-2′,3′-dihydrospiro[imidazolidine-4,1′-indene]-1-yl}acetamide;
1-amino-N-[(4S)-1-({benzyl[(1S)-1-cyclopropylethyl]carbamoyl}methyl)-2,5-dioxo-2′,3′-dihydrospiro[imidazolidine-4,1′-indene]-5′-yl]cyclopropane-1-carboxamide;
2-{5-[(azetidin-3-ylmethyl)amino]-3′,5′-dioxo-2,3-dihydrospiro[indene-1,2′-[1,4]ozazolidine]-4′-yl}-N-benzyl-N-[(1S)-1-cyclopropylethyl]acetamide;
N-benzyl-N-[(1R)-1-cyclopropyl-2,2,2-trifluoroethyl]-2-[(4S)-2,5-dioxo-2′,3′-dihydrospiro[imidazolidine-4,1′-indene]-1-yl]acetamide;
N-benzyl-N-[(1S)-1-cyclopropylethyl]-2-{5′-[(oxetan-3-ylmethyl)amino]-2,5-dioxo-2′,3′-dihydrospiro[imidazolidine-4,1′-indene]-1-yl}acetamide;
N-benzyl-N-[(1S)-1-cyclopropylethyl]-2-{5-[(1,3-dihydroxypropan-2-yl)oxy]-3′,5′-dioxo-2,3-dihydrospiro[indene-1,2′-[1,4]ozazolidine]-4′-yl}acetamide;
1-amino-N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]cyclobutane-1-carboxamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]azetidine-3-carboxamide;
4-{[4′-({benzyl[(1S)-1-cyclopropylethyl]carbamoyl}methyl)-3′,5′-dioxo-2,3-dihydrospiro[indene-1,2′-[1,4]ozazolidine]-5-ylmethyl]amino}benzoic acid;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-2,3-dihydroxypropanamide;
N-benzyl-2-{5-[(methylcarbamoyl)amino]-3′,5′-dioxo-2,3-dihydrospiro[indene-1,2′-[1,4]ozazolidine]-4′-yl}-N-(oxolan-3-yl)acetamide;
N-benzyl-N-[(1S)-1-cyclopropylethyl]-2-{5-[(1,1-dioxo-1λ⁶-thian-4-yl)amino]-3′,5′-dioxo-2,3-dihydrospiro[indene-1,2′-[1,4]ozazolidine]-4′-yl}acetamide;
N-benzyl-N-[(1S)-1-cyclopropylethyl]-2-{1,1,2′,5′-tetraoxo-2H-spiro[1λ⁶-benzothiophene-3,4′-imidazolidine]-1′-yl}acetamide;
2-{5-[(2R)-2-amino-2-cyclopropylacetamido]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
2-[5′-(azetidin-3-yloxy)-2,5-dioxo-2′,3′-dihydrospiro[imidazolidine-4,1′-indene]-1-yl]-N-benzyl-N-[(1S)-1-cyclopropylethyl]acetamide;
N-(cyclopropylmethyl)-2-{5-[(methylcarbamoyl)amino]-3′,5′-dioxo-2,3-dihydrospiro[indene-1,2′-[1,4]ozazolidine]-4′-yl}-N-[(2-methylphenyl)methyl]acetamide;
N-ethyl-2-{5-[(methylcarbamoyl)amino]-3′,5′-dioxo-2,3-dihydrospiro[indene-1,2′-[1,4]ozazolidine]-4′-yl}-N-[(2-methylphenyl)methyl]acetamide;
N-[(1S)-1-cyclopropylethyl]-2-{3′,5′-dioxo-5-[(pyrrolidin-3-yl)amino]-2,3-dihydrospiro[indene-1,2′-[1,4]ozazolidine]-4′-yl}-N-[(4-fluorophenyl)methyl]acetamide;
2-(5-{[(1-aminocyclopropyl)methyl]amino}-3′,5′-dioxo-2,3-dihydrospiro[indene-1,2′-[1,4]ozazolidine]-4′-yl)-N-[(1S)-1-cyclopropylethyl]-N-[(4-fluorophenyl)methyl]acetamide;
N-benzyl-2-{5-[(methylcarbamoyl)amino]-3′,5′-dioxo-2,3-dihydrospiro[indene-1,2′-[1,4]ozazolidine]-4′-yl}-N-(oxan-4-yl)acetamide;
N-benzyl-N-[(1R)-1-cyclopropyl-2,2,2-trifluoroethyl]-2-{5-[(methylcarbamoyl)amino]-3′,5′-dioxo-2,3-dihydrospiro[indene-1,2′-[1,4]ozazolidine]-4′-yl}acetamide;
N-benzyl-2-{5-[(methylcarbamoyl)amino]-3′,5′-dioxo-2,3-dihydrospiro[indene-1,2′-[1,4]ozazolidine]-4′-yl}-N-(oxan-3-yl)acetamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]ethanediamide;
2-{5-[(azetidin-3-yl)amino]-3,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-benzyl-N-[(1S)-1-cyclopropylethyl]-2-{3′,5′-dioxo-5-[(2-oxopyrrolidin-3-yl)amino]-2,3-dihydrospiro[indene-1,2′-[1,4]ozazolidine]-4′-yl}acetamide;
2-{[4′-({benzyl[(1S)-1-cyclopropylethyl]carbamoyl}methyl)-3′,5′-dioxo-2,3-dihydrospiro[indene-1,2′-[1,4]ozazolidine]-5-yl]carbamoyl}-2,2-dimethylacetic acid;
N-benzyl-N-[(1S)-1-cyclopropylethyl]-2-{5-[(2-hydroxyethyl)amino]-3,5′-dioxo-2,3-dihydrospiro[indene-1,2′-[1,4]ozazolidine]-4′-yl}acetamide;
(2R)-2-amino-N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]propanamide;
{[4′-({benzyl[(1S)-1-cyclopropylethyl]carbamoyl}methyl)-3′,5′-dioxo-2,3-dihydrospiro[indene-1,2′-[1,4]ozazolidine]-5-yl]carbamoyl}formic acid;
(2S)-2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-hydroxypropanamide;
N-[(1S)-1-cyclopropylethyl]-2-{3′,5′-dioxo-5-[(piperidin-3-yl)amino]-2,3-dihydrospiro[indene-1,2′-[1,4]ozazolidine]-4′-yl}-N-[(4-fluorophenyl)methyl]acetamide;
N-benzyl-N-[(1S)-1-cyclopropylethyl]-2-{5-[(methylcarbamoyl)amino]-3′,5′-dioxo-2,3-dihydro(3,3-²H₂)spiro[indene-1,2′-[1,4]ozazolidine]-4′-yl}acetamide;
N-[(4-fluorophenyl)methyl]-2-{5-[(methylcarbamoyl)amino]-3′,5′-dioxo-2,3-dihydrospiro[indene-1,2′-[1,4]ozazolidine]-4′-yl}-N-[(2S)-1,1,1-trifluoropropan-2-yl]acetamide;
2-amino-N-[4′-({benzyl[(1R)-1-cyclopropyl-2,2,2-trifluoroethyl]carbamoyl}methyl)-3′,5′-dioxo-2,3-dihydrospiro[indene-1,2′-[1,4]ozazolidine]-5-yl]-2-methylpropanamide;
2-{5-[(methylcarbamoyl)amino]-3′,5′-dioxo-2,3-dihydrospiro[indene-1,2′-[1,4]ozazolidine]-4′-yl}-N-[(2-methylphenyl)methyl]-N-(2-methylpropyl)acetamide;
2-{5-[(4-aminooxolan-3-yl)amino]-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-[(4-²H)benzyl]-N-[(1S)-1-cyclopropylethyl]-2-{5-[(methylcarbamoyl)amino]-3′,5′-dioxo-2,3-dihydrospiro[indene-1,2′-[1,4]ozazolidine]-4′-yl}acetamide;
N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-2-methyl-2-(methylamino)propanamide;
(2R)-2-amino-N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-benzyl-N-[(1S)-1-cyclopropylethyl]-2-[(1S)-5-[(methylcarbamoyl)amino]-3′,5′-dioxo-2,3-dihydrospiro[indene-1,2′-[1,4]ozazolidine]-4′-yl]acetamide;
N-benzyl-N-[(1S)-1-cyclopropylethyl]-2-[(1R)-5-[(methylcarbamoyl)amino]-3′,5′-dioxo-2,3-dihydrospiro[indene-1,2′-[1,4]ozazolidine]-4′-yl]acetamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-methylazetidine-3-carboxamide;
N-[4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylazetidine-3-carboxamide;
2-{5-[(2R)-2-amino-2-cyclopropylacetamido]-3′,5′-dioxo-2,3-dihydrospiro[indene-1,2′-[1,4]ozazolidine]-4′-yl}-N-[(4-fluorophenyl)methyl]-N-[(2S)-1,1,1-trifluoropropan-2-yl]acetamide;
N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylazetidine-3-carboxamide;
N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-2-(dimethylamino)-2-methylpropanamide;
(2R)-2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3-hydroxypropanamide;
N-[(2-amino-1,3-thiazol-5-yl)methyl]-N-[(1S)-1-cyclopropylethyl]-2-[(4S)-2,5-dioxo-2′,3′-dihydrospiro[imidazolidine-4,1′-indene]-1-yl]acetamide;
N-[(1S)-1-cyclopropylethyl]-N-{[2-(dimethylamino)-1,3-thiazol-5-yl]methyl}-2-[(4S)-2,5-dioxo-2′,3′-dihydrospiro[imidazolidine-4,1′-indene]-1-yl]acetamide;
N-[(1S)-1-cyclopropylethyl]-N-{[6-(dimethylamino)pyri din-3-yl]methyl}-2-[(4S)-2,5-dioxo-2′,3′-dihydrospiro[imidazolidine-4,1′-indene]-1-yl]acetamide;
2-{5-bromo-3′,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
2-{5-[(3-aminocyclohexyl)amino]-3,5′-dioxo-2,3-dihydrospiro[indene-1,2′-[1,4]ozazolidine]-4′-yl}-N-[(1S)-1-cyclopropylethyl]-N-[(4-fluorophenyl)methyl]acetamide;
N-[(2-amino-1,3-thiazol-4-yl)methyl]-N-[(1S)-1-cyclopropylethyl]-2-[(4S)-2,5-dioxo-2′,3′-dihydrospiro[imidazolidine-4,1′-indene]-1-yl]acetamide;
N-[1-(azetidin-3-yl)ethyl]-N-benzyl-2-{5-[(methylcarbamoyl)amino]-3′,5′-dioxo-2,3-dihydrospiro[indene-1,2′-[1,4]ozazolidine]-4′-yl}acetamide;
N-[(1S)-1-cyclopropylethyl]-N-[(4-fluorophenyl)methyl]-2-{1,1,2′,5′-tetraoxo-2H-spiro[1λ⁶-benzothiophene-3,4′-imidazolidine]-1′-yl}acetamide;
N-[(1S)-1-cyclopropylethyl]-2-[(4S)-2,5-dioxo-2,3-dihydrospiro[imidazolidine-4,1′-indene]-1-yl]-N-[(6-hydroxypyridin-3-yl)methyl]acetamide;
N-[(1S)-1-cyclopropylethyl]-2-[(4S)-2,5-dioxo-2′,3′-dihydrospiro[imidazolidine-4,1′-indene]-1-yl]-N-[(6-methoxypyridin-3-yl)methyl]acetamide;
2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-2-methylpropanamide;
N-[(4-fluorophenyl)methyl]-N-[(2S)-1-methoxypropan-2-yl]-2-{5-[(methylcarbamoyl)amino]-3′,5′-dioxo-2,3-dihydrospiro[indene-1,2′-[1,4]ozazolidine]-4′-yl}acetamide;
3-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]oxetane-3-carboxamide;
N-[(1S)-1-cyclopropylethyl]-N-{[2-(dimethylamino)-1,3-thiazol-4-yl]methyl}-2-[(4 S)-2,5-dioxo-2′,3′-dihydrospiro[imidazolidine-4,1′-indene]-1-yl]acetamide;
N-benzyl-N-[(1R)-1-cyclopropyl-2,2,2-trifluoroethyl]-2-{1,1,2′,5′-tetraoxo-2H-spiro[1λ⁶-benzothiophene-3,4′-imidazolidine]-1′-yl}acetamide;
N-[(6-amino-5-methoxypyridin-3-yl)methyl]-N-[(1S)-1-cyclopropylethyl]-2-[(4S)-2,5-dioxo-2′,3′-dihydrospiro[imidazolidine-4,1′-indene]-1-yl]acetamide;
N-[(6-aminopyridin-3-yl)methyl]-N-[(1S)-1-cyclopropylethyl]-2-[(4S)-2,5-dioxo-2,3′-dihydrospiro[imidazolidine-4,1′-indene]-1-yl]acetamide;
N-benzyl-2-{6-bromo-1,1,2′,5′-tetraoxo-2H-spiro[1λ⁶-benzothiophene-3,4′-imidazolidine]-1′-yl}-N-[(1S)-1-cyclopropylethyl]acetamide;
N-benzyl-N-[(1S)-1-cyclopropylethyl]-2-[1,1,2′,5′-tetraoxo-6-(1H-pyrazol-3-yl)-2H-spiro[1λ⁶-benzothiophene-3,4′-imidazolidine]-1′-yl]acetamide;
2-amino-N-[(1S)-4′-({[(4-bromophenyl)methyl][(1S)-1-cyclopropylethyl]carbamoyl}methyl)-3′,5′-dioxo-2,3-dihydrospiro[indene-1,2′-[1,4]ozazolidine]-5-yl]-2-methylpropanamide;
2-amino-N-[(1R)-4′-({[(4-bromophenyl)methyl][(1S)-1-cyclopropylethyl]carbamoyl}methyl)-3′,5′-dioxo-2,3-dihydrospiro[indene-1,2′-[1,4]ozazolidine]-5-yl]-2-methylpropanamide;
2-{5-[(2R)-2-amino-2-(oxetan-3-yl)acetamido]-3′,5′-dioxo-2,3-dihydrospiro[indene-1,2′-[1,4]ozazolidine]-4′-yl}-N-benzyl-N-[(1S)-1-cyclopropylethyl]acetamide;
1-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3-difluorocyclobutane-1-carboxamide;
2-amino-N-[4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1S)-4′-({[(1S)-1-cyclopropylethyl][(4-fluorophenyl)methyl]carbamnoyl}methyl)-3′,5′-dioxo-2,3-dihydrospiro[indene-1,2′-[1,4]ozazolidine]-5-yl]-3-methylbutanamide;
(2R)-2-amino-N-[(1R)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]-N-[(2S)-1,1,1-trifluoropropan-2-yl]acetamide;
N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]-N-[(2S)-1,1,1,1-trifluoropropan-2-yl]acetamide;
N-[(1S)-1-cyclopropylethyl]-N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]acetamide;
N-[(1S)-1-cyclopropylethyl]-N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]acetamide;
(2S)-2-amino-N-[(1S)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1S)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2S)-2-amino-N-[(1R)-4′-({benzyl[(1S)-1-cyclopropylethyl]carbamoyl}methyl)-3′,5′-dioxo-2,3-dihydrospiro[indene-1,2′-[1,4]ozazolidine]-5-yl]-3,3,3-trifluoropropanamide;
(2R)-2-amino-N-[(1R)-4′-({benzyl[(1S)-1-cyclopropylethyl]carbamoyl}methyl)-3′,5′-dioxo-2,3-dihydrospiro[indene-1,24′-[1,4]ozazolidine]-5-yl]-3,3,3-trifluoropropanamide;
N-benzyl-N-[(1R)-1-cyclopropyl-2,2,2-trifluoroethyl]-2-[(1S)-5-[(methylcarbamoyl)amino]-3′,5′-dioxo-2,3-dihydrospiro[indene-1,2′-[1,4]ozazolidine]-4′-yl]acetamide;
N-benzyl-N-[(1R)-1-cyclopropyl-2,2,2-trifluoroethyl]-2-[(1R)-5-[(methylcarbamoyl)amino]-3′,5′-dioxo-2,3-dihydrospiro[indene-1,2′-[1,4]ozazolidine]-4′-yl]acetamide;
(2R)-2-amino-N-[(1S)-4′-({[(4-fluorophenyl)methyl][(2S)-1,1, 1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1R)-4′-({[(4-fluorophenyl)methyl][(2S)-1,1,1-trifluoropropan-2-yl]carbamoyl}ethyl)-3′,5′-dioxo-2,3-dihydrospiro[indene-1,2′-[1,4]ozazolidine]-5-yl]-3-methylbutanamide;
(2R)-2-amino-N-[(1S)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
(2R)-2-amino-N-[(1R)-4′-({[(1R)-1-cyclopropyl-2,2,2-trifluoroethyl][(4-fluorophenyl)methyl]carbamnoyl}methyl)-3′,5′-dioxo-2,3-dihydrospiro[indene-1,2′-[1,4]ozazolidine]-5-yl]-3-methylbutanamide;
N-[(1R)-1-cyclopropyl-2,2,2-trifluoroethyl]-N-[(4-fluorophenyl)methyl]-2-[(1S)-5-[(methylcarbamoyl)amino]-3′,5′-dioxo-2,3-dihydrospiro[indene-1,2′-[1,4]ozazolidine]-4′-yl]acetamide;
N-[(1R)-1-cyclopropyl-2,2,2-trifluoroethyl]-N-[(4-fluorophenyl)methyl]-2-[(1R)-5-[(methylcarbamoyl)amino]-3′,5′-dioxo-2,3-dihydrospiro[indene-1,2′-[1,4]ozazolidine]-4′-yl]acetamide;
N-[(1S)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
N-[(1R)-4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
(2R)-2-amino-N-[4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
N-[(1S)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide
N-[(1R)-4′-({[(1S)-1-cyclopropylethyl][(4-fluorophenyl)methyl]carbamoyl}methyl)-3′,5′-dioxo-2,3-dihydrospiro[indene-1,2′-[1,4]ozazolidine]-5-yl]azetidine-3-carboxamide;
(2S)-2-amino-N-[4′-({[(1R)-1-cyclopropyl-2,2,2-trifluoroethyl][(4-fluorophenyl)methyl]carbamoyl}methyl)-3′,5′-dioxo-2,3-dihydrospiro[indene-1,2′-[1,4]ozazolidine]-5-yl]-3-methylbutanamide;
(2S)-2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
(2R)-2-amino-N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]-3-methylbutanamide;
N-[4′-({[(4-fluorophenyl)methyl][(2S)-1,1,1-trifluoropropan-2-yl]carbamoyl}methyl)-3′,5′-dioxo-2,3-dihydrospiro[indene-1,2′-[1,4]ozazolidine]-5-yl]azetidine-3-carboxamide;
N-benzyl-N-[1-(1-methylazetidin-3-yl)ethyl]-2-{5-[(methylcarbamoyl)amino]-3′,5′-dioxo-2,3-dihydrospiro[indene-1,2′-[1,4]ozazolidine]-4′-yl}acetamide;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1R)-3′-(2-{[(1R)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-benzyl-2-[(1R)-5-bromo-2′,4′-dioxo-2,3-dihydro-3H-spiro[indene-1,5′-[1,3]ozazolidin]-3′-yl]-N-[(1S)-1-cyclopropylethyl]acetamide;
2-[(1S)-5-amino-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-[(1R)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-[(1S)-3′-(2-{[(1R)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-[(1R)-1-cyclopropylethyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-(5′-bromo-2,5-dioxo-1′,3′-dihydro-1H-spiro[imidazolidine-4,2′-inden]-1-yl)-N-[(1S)-1-cyclopropylethyl]-N-(4-fluorobenzyl)acetamide;
2-(6-bromo-1,1-dioxido-2′,5′-dioxo-1′H-spiro[1-benzothiophene-3,4′-imidazolidin]-1′-yl)-N-[(1S)-1-cyclopropylethyl]-N-(4-fluorobenzyl)acetamide;
N-[(1R)-1-cyclopropylethyl]-N-(4-fluorobenzyl)-2-{(1S)-5-[(methylcarbamoyl)amino]-2′,4′-dioxo-2,3-dihydro-3′H-spiro[indene-1,5′-[1,3]ozazolidin]-3′-yl}acetamide;
N-[(1R)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
N-[(1S)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-3,3-difluoroazetidine-1-carboxamide;
tert-butyl 3-[(1R)-3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]azetidine-1-carboxylate;
N-[(1S)-1-cyclopropylethyl]-2-[2,5-dioxo-5′-(6-oxo-1,6-dihydropyridin-3-yl)-1′,3′-dihydro-1H-spiro[imidazolidine-4,2′-inden]-1-yl]-N-(4-fluorobenzyl)acetamide;
2-(5′-amino-2,5-dioxo-1′,3′-dihydro-1H-spiro[imidazolidine-4,2′-inden]-1-yl)-N-[(1S)-1-cyclopropylethyl]-N-(4-fluorobenzyl)acetamide;
2-[(1R)-5-(6-aminopyridin-3-yl)-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
2-{(1R)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-[(1S)-1-cyclopropylethyl]-N-(4-fluorobenzyl)acetamide;
N-[(1S)-1-cyclopropylethyl]-2-[(1R)-2′,4′-dioxo-5-(pyridin-3-yl)-2,3-dihydro-3′H-spiro[indene-1,5′-[1,3]ozazolidin]-3′-yl]-N-(4-fluorobenzyl)acetamide;
N-[(1S)-1-cyclopropylethyl]-2-[1,1-dioxido-2′,5′-dioxo-6-(6-oxo-1,6-dihydropyridin-3-yl)-1′H-spiro[1-benzothiophene-3,4′-imidazolidin]-1′-yl]-N-(4-fluorobenzyl)acetamide;
N-benzyl-2-(7-bromo-2′,4′-dioxo-2,3-dihydro-3′H-spiro[chromene-4,5′-[1,3]ozazolidin]-3′-yl)-N-[(1S)-1-cyclopropylethyl]acetamide;
2-[(1R)-5-(azetidin-3-yl)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
2-[(1R)-5-(1-acetylazetidin-3-yl)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-benzyl-N-[(1S)-1-cyclopropylethyl]-2-[(1R)-5-(1-methylazetidin-3-yl)-2′,4′-dioxo-2,3-dihydro-3′H-spiro[indene-1,5′-[1,3]ozazolidin]-3′-yl]acetamide;
3-[(1R)-3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]-N-methylazetidine-1-carboxamide;
N-benzyl-2-{(1R)-5-[1-(cyclopropylcarbonyl)azetidin-3-yl]-2′,4′-dioxo-2,3-dihydro-3′H-spiro[indene-1,5′-[1,3]ozazolidin]-3′-yl}-N-[(1S)-1-cyclopropylethyl]acetamide;
N-benzyl-N-[(1S)-1-cyclopropylethyl]-2-{(1R)-5-[1-(2-hydroxy-2-methylpropanoyl)azetidin-3-yl]-2′,4′-dioxo-2,3-dihydro-3′H-spiro[indene-1,5′-[1,3]ozazolidin]-3′-yl}acetamide;
N-benzyl-N-[(1S)-1-cyclopropylethyl]-2-{(1R)-5-[1-(methylsulfonyl)azetidin-3-yl]-2′,4′-dioxo-2,3-dihydro-3′H-spiro[indene-1,5′-[1,3]ozazolidin]-3′-yl}acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]cyclobutanecarboxamide;
3-ethoxy-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-5-oxo-D-prolinamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-5-oxo-L-prolinamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropane-2-yl]amino}-2-oxoethyl)-2′,4′-dioxo-2,3-dihydrospiro[indene-1,5′-[1,3]ozazolidin]-5-yl]cyclopropanecarboxamide;
N-(4-fluorobenzyl)-2-{(1R)-5-[(methoxyacetyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(1R)-5-[(ethoxyacetyl)amino]-2′,4′-dioxo-2,3-dihydro-3′H-spiro[indene-1,5-[1,3]ozazolidin]-3′-yl}-N-(4-fluorobenzyl)-N-[(2S)-1,1,1-trifluoropropan-2-yl]acetamide;
3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]propanamide;
N-[(1R)-3′-(2-{(4-fluorobenzyl)[(2S)-1,1,1-trifluoropropane-2-yl]amino}-2-oxoethyl)-2′,4′-dioxo-2,3-dihydrospiro[indene-1,5′-[1,3]ozazolidin]-5-yl]-3-methylbutanamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylpropanamide;
2-[(1R)-5-(acetylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(7-amino-2′,4′-dioxo-2,3-dihydro-3′H-spiro[chromene-4,5′-[1,3]ozazolidin]-3′-yl)-N-benzyl-N-[(1S)-1-cyclopropylethyl]acetamide;
2-(6-amino-1,1-dioxido-2′,5′-dioxo-1′H-spiro[1-benzothiophene-3,4′-imidazolidin]-1′-yl)-N-[(1S)-1-cyclopropylethyl]-N-(4-fluorobenzyl)acetamide;
N-[(1S)-1-cyclopropylethyl]-N-(4-fluorobenzyl)-2-{5′-[(methylcarbamoyl)amino]-2,5-dioxo-1′,3′-dihydro-1H-spiro[imidazolidine-4,2′-inden]-1-yl}acetamide;
N-(4-fluorobenzyl)-2-[(1R)-5-{[(2-methoxyethyl)(methyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[(dimethylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-{[methyl(2-methylpropyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(cyclopropylmethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(cyclobutylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-({[(2R)-1-hydroxy-3-methylbutan-2-yl]carbamoyl}amino)-2′,4′-dioxo-2,3-dihydro-3′H-spiro[indene-1,5′-[1,3]ozazolidin]-3′-yl]-N-[(2S)-1,1,1-trifluoropropane-2-yl]acetamide;
2-[(1R)-2′,4′-dioxo-5-({[(2S)-tetrahydrofuran-2-ylmethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-({[(2R)-tetrahydrofuran-2-ylmethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-({[2-(propan-2-yloxy)ethyl]carbamoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(cyclopropylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-{[ethyl(methyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[(diethylcarbamoyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyrrolidine-1-carboxamide;
2-[(1R)-5-({[2-(dimethylamino)ethyl]methyl)carbamoyl}amino)-2′,4′-dioxo-2′,3′-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N²-{([(1R)-3′-(2-{(4-fluorobenzyl) [(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-L-leucinamide;
N-(4-fluorobenzyl)-2-[(1R)-5-{[(2-hydroxyethyl)(propyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(2-methoxyethyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(3-hydroxypropyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-({[(2S)-1-hydroxy-3-methylbutan-2-yl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[methyl(propan-2-yl)carbamoyl]amino}-2′,4′-dioxo-2,3-dihydro-3′1-spiro[indene-1,5′-[1,3]ozazolidin]-3′-yl]-N-[(2S)-1,1,1-trifluoropropan-2-yl]acetamide;
2-[(1R)-5-{[(2-cyanoethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-{[methyl(propyl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[5′-(acetylamino)-2,5-dioxo-1′,3′-dihydro-1H-spiro[imidazolidine-4,2′-inden]-1-yl]-N-[(1S)-1-cyclopropylethyl]-N-(4-fluorobenzyl)acetamide;
N-[(1S)-1-cyclopropylethyl]-N-(4-fluorobenzyl)-2-{6-[(methylcarbamoyl)amino]-1,1-dioxido-2′,5′-dioxo-1′H-spiro[1-benzothiophene-3,4′-imidazolidin]-1′-yl}acetamide;
N-(4-fluorobenzyl)-2-(1′-methyl-2,5-dioxo-2′,3′-dihydro-1,1′H-spiro[imidazolidine-4,4′-quinolin]-1-yl)-N-[(2S)-1,1,1-trifluoropropan-2-yl]acetamide;
N-benzyl-N-[(1S)-1-cyclopropylethyl]-2-{7-[(methylcarbamoyl)amino]-2′,4′-dioxo-2,3-dihydro-3′H-spiro[chromene-4,5′-[1,3]ozazolidin]-3′-yl}acetamide;
2-[7-(acetylamino)-2′,4′-dioxo-2,3-dihydro-3′H-spiro[chromene-4,5′-[1,3]ozazolidin]-3′-yl]-N-benzyl-N-[(1S)-1-cyclopropylethyl]acetamide;
N-[(1S)-1-cyclopropylethyl]-N-(4-fluorobenzyl)-2-(3′-hydroxy-2,5-dioxo-2′,3′-dihydro-1H-spiro[imidazolidine-4,1′-inden]-1-yl)acetamide;
N-[(1S)-1-cyclopropylethyl]-2-[(1R)-2′,4′-dioxo-5-(pyridin-4-yl)-2,3-dihydro-3′H-spiro[indene-1,5′-[1,3]ozazolidin]-3′-yl]-N-(4-fluorobenzyl)acetamide;
N-[(1S)-1-cyclopropylethyl]-N-(4-fluorobenzyl)-2-[(1R)-5-(6-hydroxypyridazin-3-yl)-2′,4′-dioxo-2,3-dihydro-3′H-spiro[indene-1,5′-[1,3]ozazolidin]-3′-yl]acetamide;
N-[(1S)-1-cyclopropylethyl]-2-[(1R)-2′,4′-dioxo-5-(1H-pyrazol-4-yl)-2,3-dihydro-3′H-spiro[indene-1,5′-[1,3]ozazolidin]-3′-yl]-N-(4-fluorobenzyl)acetamide;
N-(4-fluorobenzyl)-N-[(2S)-1,1,1-trifluoropropan-2-yl]-2-(2,3′,5-trioxo-2′,3′-dihydro-1H-spiro[imidazolidine-4,1′-inden]-1-yl)acetamide;
N-[(1S)-1-cyclopropylethyl]-N-(4-fluorobenzyl)-2-(1-methyl-2,5-dioxo-2′,3′-dihydro-1H,1′H-spiro[imidazolidine-4,4′-quinolin]-1-yl)acetamide
N-(4-fluorobenzyl)-2-[(3′E)-3′-(hydroxyimino)-2,5-dioxo-2′,3′-dihydro-1H-spiro[imidazolidine-4,1′-inden]-1-yl]-N-[(2S)-1,1,1-trifluoropropan-2-yl]acetamide;
N-[(1S)-1-cyclopropylethyl]-N-(4-fluorobenzyl)-2-(2,3′,5-trioxo-2′,3′-dihydro-1H-spiro[imidazolidine-4,1′-inden]-1-yl)acetamide;
N-benzyl-N-[(1S)-1-cyclopropylethyl]-2-[2′,4′-dioxo-7-(6-oxo-1,6-dihydropyridin-3-yl)-2,3-dihydro-3′H-spiro[chromene-4,5′-[1,3]ozazolidin]-3′-yl]acetamide;
N-(4-fluorobenzyl)-2-(3′-hydroxy-2,5-dioxo-2′,3′-dihydro-1H-spiro[imidazolidine-4,1′-inden]-1-yl)-N-[(2S)-1,1,1-trifluoropropan-2-yl]acetamide;
2-{(1R)-2′,4′-dioxo-5-[(tetrahydrofuran-2-ylmethyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[(cyclopentylmethyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[(2,2-dimethylbutyl)amino]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-2′,4′-dioxo-5-[(tetrahydrofuran-3-ylmethyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-{(1R)-5-[(2-methylpropyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-[6-(acetylamino)-1,1-dioxido-2′,5′-dioxo-1′H-spiro[1-benzothiophene-3,4′-imidazolidin]-1′-yl]-N-[(1S)-1-cyclopropylethyl]-N-(4-fluorobenzyl)acetamide;
tert-butyl [(2R)-1-{[3′-(2-{benzyl[(1S)-1-cyclopropylethyl]amino}-2-oxoethyl)-2′,4′-dioxo-2,3-dihydrospiro[chromene-4,5′-[1,3]ozazolidin]-7-yl]amino}-3-methyl-1-oxobutan-2-yl]carbamate;
N-[3′-(2-{benzyl[(1S)-1-cyclopropylethyl]amino}-2-oxoethyl)-2′,4′-dioxo-2,3-dihydrospiro[chromene-4,5′-[1,3]ozazolidin]-7-yl]-D-valinamide;
2-[(1R)-6-amino-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
2-[(1R)-6-(acetylamino)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-benzyl-N-[(1S)-1-cyclopropylethyl]-2-{(1R)-6-[(methylcarbamoyl)amino]-2′,4′-dioxo-2,3-dihydro-3′H-spiro[indene-1,5′-[1,3]ozazolidin]-3′-yl}acetamide;
2-[(1S)-6-amino-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
2-[(1S)-6-(acetylamino)-2′,4′-dioxo-2,3-dihydro-3′H-spiro[indene-1,5′-[1,3]ozazolidin]-3′-yl]-N-benzyl-N-[(1S)-1-cyclopropylethyl]acetamide;
N-benzyl-N-[(1S)-1-cyclopropylethyl]-2-{(1S)-6-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-2-[(1R)-2′,4′-dioxo-5-(pyridin-2-yl)-2,3-dihydro-3′H-spiro[indene-1,5′-[1,3]ozazolidin]-3′-yl]-N-(4-fluorobenzyl)acetamide;
2-(7′-bromo-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
N-[(1S)-1-cyclopropylethyl]-N-(3-fluorobenzyl)-2-{(1R)-5-[(methylcarbamoyl)amino]-2,4-dioxo-2,3-dihydro-3′H-spiro[indene-1,5′-[1,3]ozazolidin]-3′-yl}acetamide;
N-[(1S)-1-cyclopropylethyl]-N-(3,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-(3′-fluoro-2,5-dioxo-2′,3′-dihydro-1H-spiro[imidazolidine-4,1′-inden]-1-yl)-N-[(2S)-1,1,1-trifluoropropan-2-yl]acetamide;
2-(3′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[3′-(methylamino)-2,5-dioxo-2′,3′-dihydro-1H-spiro[imidazolidine-4,1′-inden]-1-yl]-N-[(2S)-1,1,1-trifluoropropan-2-yl]acetamide;
N-[(1S)-1-cyclopropyl-2,2,2-trifluoroethyl]-N-(4-fluorobenzyl)-2-{7′-[(methylcarbamoyl)amino]-2,4-dioxo-2′,3′-dihydro-3H-spiro[1,3-ozazolidine-5,4′-thiochromen]-3-yl}acetamide;
2-[(1R)-5-(carbamoylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(7′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
N-[(1S)-1-cyclopropyl-2,2,2-trifluoroethyl]-N-(4-fluorobenzyl)-2-{7′-[(methylcarbamoyl)amino]-1′,1′-dioxido-2,4-dioxo-2′,3′-dihydro-3H-spiro[1,3-ozazolidine-5,4′-thiochromen]-3-yl}acetamide;
2-(6-bromo-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide;
N-(4-fluorobenzyl)-2-((R)-5-(3-(N-methylsulfamoyl)ureido)-2′,4′-dioxo-2,3-dihydrospiro[indene-1,5′-ozazolidine]-3′-yl)-N—((S)-1,1,1-trifluoropropan-2-yl)acetamide
methyl {[(1R)-3′-(2-{(4-fluorobenzyl)[(2)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}sulfamate;
2-((R)-5-(3-(N-(cyclopropylmethyl)sulfamoyl)ureido)-2′,4′-dioxo-2,3-dihydrospiro[indene-1,5′-ozazolidine]-3′-yl)-N-(4-fluorobenzyl)-N—((S)-1,1,1-trifluoropropan-2-yl)acetamide
N-[(1S)-1-cyclopropylethyl]-N-(4-fluorobenzyl)-2-{7′-[(methylcarbamoyl)amino]-2,5-dioxo-3′,4′-dihydro-1H,1′H-spiro[imidazolidine-4,2′-naphthalene]-1-yl}acetamide;
2-[7′-(acetylamino)-2,5-dioxo-3′,4′-dihydro-1H,1′H-spiro[imidazolidine-4,2′-naphthalen]-1-yl]-N-[(1S)-1-cyclopropylethyl]-N-(4-fluorobenzyl)acetamide;
(1R)-3′-(2-{benzyl[(1S)-1-cyclopropylethyl]amino}-2-oxoethyl)-N-(2-n-methylpropyl)-2′,4′-dioxo-2,3-dihydrospiro[indene-1,5′-[1,3]ozazolidine]-5-carboxamide;
N-[(1S)-1-cyclopropylethyl]-N-(2-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,3-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide
N-(2-chloro-4-fluorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(3-chloro-4-fluorobenzyl)-N-[(1S)-1-cyclopropylethyl]-2-{(1R)-5-[(methylcarbamoyl)amino]2′,4′-dioxo-2,3-dihydro-3′H-spiro[indene-1,5′-[1,3]ozazolidin]-3′-yl}acetamide;
N-(4-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-[4-(trifluoromethyl)benzyl]acetamide
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1-methyl-1H-pyrazole-4-carboxamide
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1-methyl-1H-pyrazole-3-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-4-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-2-carboxamide;
2-(5-bromo-4-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
2-(5-bromo-6-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
(1R)-3′-(2-{benzyl[(1S)-1-cyclopropylethyl]amino}-2-oxoethyl)-N-[(2R)-1-hydroxy-3-methylbutan-2-yl]-2′,4′-dioxo-2,3-dihydrospiro[indene-1,5′-[1,3]ozazolidine]-5-carboxamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-[(5-methyl-1,3,4-thiadiazol-2-yl)methyl]acetamide;
N-(4-fluorobenzyl)-2-(2′-methyl-2,5-dioxo-2′,3′-dihydro-1H-spiro[imidazolidine-4,1′-inden]-1-yl)-N-[(2S)-1,1,1-trifluoropropan-2-yl]acetamide;
2-(5-amino-4-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{4-fluoro-5-[(methylcarbamoyl)amino]-2′,4′-dioxo-2,3-dihydro-3′H-spiro[indene-1,5′-[1,3]ozazolidin]-3′-yl}acetamide;
N-(4-cyanobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3-methylbenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3,5-difluorobenzyl)-2-{(1R)-5-[(methylcarbamoyl)amino]-2′,4′-dioxo-2,3-dihydro-3′H-spiro[indene-1,5′-[1,3]ozazolidin]-3′-1}acetamide;
N-(3-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-chlorobenzyl)-N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(2,4-dichlorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-methoxybenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(3-methoxybenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropylethyl]-N-(4-methylbenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-[(1R)-5-{[(1-methyl-1H-pyrazol-4-yl)carbamoyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-[1-(2-{[(1S)-1-cyclopropylethyl](4-fluorobenzyl)amino}-2-oxoethyl)-2,5-dioxo-3,4′-dihydro-1′H-spiro[imidazolidine-4,2′-naphthalen]-7′-yl]-5-oxo-D-prolinamide;
2-{7-[1-(2-amino-2-oxoethyl)-H-pyrazol-4-yl]-2′,4′-dioxo-2,3-dihydro-3′H-spiro[chromene-4,5′-[1,3]ozazolidin]-3′-yl}-N-benzyl-N-[(1S)-1-cyclopropylethyl]acetamide
2-{7′-[1-(2-amino-2-oxoethyl)-1H-pyrazol-4-yl]-2,5-dioxo-3′,4′-dihydro-1H,1′H-spiro[imidazolidine-4,2′-naphthalen]-1-yl}-N-[(1S)-1-cyclopropylethyl]-N-(4-fluorobenzyl)acetamide;
2-{(1R)-2′,4′-dioxo-5-[(phenylcarbamoyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-2′,4′-dioxo-5-[(pyridin-3-ylcarbamoyl)amino]-2,3-dihydro-3′H-spiro[indene-1,5′-[1,3]ozazolidin]-3′-yl}-N-(4-fluorobenzyl)-N-[(2S)-1,1-trifluoropropan-2-yl]acetamide;
2-(5′-bromo-2,3′,5′-trioxo-2′,3′-dihydro-1-spiro[imidazolidine-4,1′-inden]-1-yl)-N-(4-fluorobenzyl)-N-[(2)-1,1,1-trifluoropropan-2-yl]acetamide;
2-(5-amino-6-fluoro-2′,4′-dioxo-2,3-dihydro-3′H-spiro[indene-1,5′-[1,3]ozazolidin]-3′-yl)-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{6-fluoro-5-[(methylcarbamoyl)amino]-2′,4′-dioxo-2,3-dihydro-3′I-spiro[indene-1,5′-[1,3]ozazolidin]-3′-yl}acetamide;
N-[(1S)-1-cyclopropylethyl]-N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetamide;
N-(4-fluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]N-[(2S)-1,1,1-trifluoropropan-2-yl]acetamide;
2-[(1R)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[1-(2-amino-2-oxoethyl)-1H-pyrazol-4-yl]-2′,4′-dioxo-2,3-dihyd H-spiro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(dimethylamino)pyridin-2-yl]-2′,4′-dioxo-2,3-dihydro-3′H-spiro[indene 1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(4-methylpyrimidin-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[5-(difluoromethyl)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(6-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1R)-5-[1-(2-methylpropyl)-1H-pyrazol-4-yl]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(1R)-5-[4-(difluoromethyl)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(3-fluoropyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(1,3-thiazol-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-N-methylbenzamide;
2-{(1R)-5-[2-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(5-cyanopyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(1-methyl-1H-1,2,4-triazol-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(5-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-(6-cyanopyridin-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(5-fluoro-3-methylpyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(5-methyl-1,3,4-thiadiazol-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(4-methoxypyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(6-methoxypyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[2-(dimethylamino)pyrimidin-5-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(acetylamino)pyridin-3-yl]-2′,4′-diox-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(1-methyl-1H-1,2, 4-triazol-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-cyclopropyl-5-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]furan-2-carboxamide;
N-(4-fluorobenzyl)-2-[(1R)-5-(5-fluoropyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
4-fluoro-3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
5-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene 1-5′-[1,3]ozazolidin]-5-yl]-N-methylpyridine-2-carboxamide;
6-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-3-carboxamide;
N-(4-fluorobenzyl)-2-[(1R)-5-(2-methoxypyrimidin-5-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
6-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-2-carboxamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]thiophene-3-carboxamide
2-{(1R)-5-[4-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(1,3-dimethyl-2,4-dioxo-1,2,3,4-tetrahydropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropropan-2-yl]acetamide;
2-[(1R)-5-(4-cyanopyridin-2-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]pyridine-4-carboxamide;
2-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
4-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
2-{(1R)-5-[3-(acetylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(6-cyano-5-methoxypyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(1-methyl-1H-pyrazol-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-2′,4′-dioxo-5-(1,3,5-trimethyl-1H-pyrazol-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(3,4-difluorophenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2,5-difluoro-4-ethoxyphenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyrimidin-5-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-2′,4′-dioxo-5-[5-(trifluoromethyl)pyridin-3-yl]-2,3-dihydro-3′H-spiro[indene-1, 5′-[1,3]ozazolidin]-3′-yl}-N-(4-fluorobenzyl)-N-[(2S)-1,1,1-trifluoropropan-2-yl]acetamide;
2-[(1R)-2′,4′-dioxo-5-(1,3-thiazol-2-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2-cyanopyridin-4-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyrazin-2-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[4-(carbamoylamino)phenyl]-2′,4′-dioxo-2, 3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(2-cyanopyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(6-cyano-5-fluoropyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]benzamide;
N-(4-fluorobenzyl)-N-[(2S)-1,1,1-trifluoropropan-2-yl]-2-(2,2′,5-trioxo-2′,3′-dihydro-1H,1′H-spiro[imidazolidine-4,4′-quinolin]-1-yl)acetamide;
2-(2,5-dioxo-1H-spiro[imidazolidine-4,1′-inden]-1-yl)-N-(4-fluorobenzyl)-N-[(2S)-1,1,1-trifluoropropan-2-yl]acetamide;
2-{5-[1-(2-amino-2-oxoethyl)-1H-pyrazol-4-yl]-2,3′,5-trioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-N-[(2S)-1,1,1-trifluoropropan-2-yl]-2-[2,3′,5-trioxo-5′-(6-oxo-1,6-dihydropyridin-3-yl)-2′,3′-dihydro-1H-spiro[imidazolidine-4,1′-inden]-1-yl]acetamide;
2-[(1R)-5-{([(4-aminophenyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1S)-1-cyclopropylethyl]-2-[(4R)-2,5-dioxo-2′,3′-dihydro-1H-spiro[imidazolidine-4,1′-inden]-1-yl]-N-(4-fluorobenzyl)acetamide;
N-[(1S)-1-cyclopropylethyl]-2-[(4S)-2,5-dioxo-2′,3′-dihydro-1H-spiro[imidazolidine-4,1′-inden]-1-yl]-N-(4-fluorobenzyl)acetamide;
2-(7′-bromo-1′-methyl-2,5-dioxo-2′,3′-dihydro-1H,1′H-spiro[imidazolidine-4,4′-quinolin]-1-yl)-N-[(1S)-1-cyclopropylethyl]-N-(4-fluorobenzyl)acetamide;
N-(4-fluorobenzyl)-2-{6-[(methylcarbamoyl)amino]-2′,4′-dioxo-3,4-dihydro-2H,3′H-spiro[naphthalene-1,5′-[1,3]ozazolidin]-3′-yl}-N-[(2S)-1,1,1-trifluoropropan-2-yl]acetamide;
2-[5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(pyrimidin-5-ylmethyl)acetamide;
N-[(1S)-1-cyclopropylethyl]-2-{(1R)-5-[(methylcarbamoyl)amino]-2′,4′-dioxo-2′,3′-dihydro-3′H-spiro[indene-1,5′-[1,3]ozazolidin]-3′-yl}-N-(pyrimidin-2-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-thiazol-5-ylmethyl)acetamide;
N-(4-fluorobenzyl)-2-(4′-hydroxy-2,5-dioxo-3′,4′-dihydro-1-H,2′H-spiro[imidazolidine-4,1′-naphtlalen]-1-yl)-N-[(2S)-1,1,1-trifluoropropan-2-yl]acetamide;
2-(6-{1-[2-(dimethylamino)-2-oxoethyl]-1H-pyrazol-4-yl}-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide;
2-[5′-(acetylamino)-2,3′,5-trioxo-2′,3′-dihydro-H-spiro[imidazolidine-4,1′-inden]-1-yl]-N-(4-fluorobenzyl)-N-[(2S)-1,1,1-trifluoropropan-2-yl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]prolinamide;
N²-(4-fluorobenzyl)-N²-({(1R)-5-[(methylcarbamoyl)amino]-2′,4′-dioxo-2,3-dihydro-3′H-spiro[indene-1,5′-[1,3]ozazolidin]-3′-yl}acetyl)-L-alaninamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-1H-pyrazole-5-carboxamide;
2-[5′-(acetylamino)-2,5-dioxo-2′,3′-dihydro-1H-spiro[imidazolidine-4,1′-inden]-1-yl]-N-[(1S)-1-cyclopropylethyl]-N-(4-fluorobenzyl)acetamide;
N-(4-fluorobenzyl)-2-[(1R)-5-{[(methylsulfamoyl)acetyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-{[(methylsulfonyl)acetyl]amino}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1R)-5-{[(dimethylsulfamoyl)acetyl]amino}-2′,4′-dioxo-2,3-dihydro-3′H-spiro[indene-1,5-[1,3]ozazolidin]-3′-yl]-N-(4-fluorobenzyl)-N-[(2S)-1,1,1-trifluoropropan-2-yl]acetamide;
2-{(1R)-2′,4′-dioxo-5-[(sulfamoylacetyl)amino]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-(5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide;
(3R)-3-[(4-fluorobenzyl)({(1R)-5-[(methylcarbonyl)amino]-2′,4′-dioxo-2,3-dihydro-3′H-spiro[indene-1,5′-[1,3]ozazolidin]-3′-yl}acetyl)amino]pyrrolidine-1-carboxamide
N-benzyl-N-ethyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-(3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-(3′-fluoro-5-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
2-(6′-bromo-2,5-dioxo-3′,4′-dihydro-1H, 1′H-spiro[imidazolidine-4,2′-naphthalen]-1-yl)-N-[(1S)-1-cyclopropylethyl]-N-(4-fluorobenzyl)acetamide;
methyl N′-cyano-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamimidothioate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanamide;
2-(6′-amino-2,5-dioxo-3′,4′-dihydro-1H,1′H-spiro[imidazolidine-4,2′-naphthalen]-1-yl)-N-[(1S)-1-cyclopropylethyl]-N-(4-fluorobenzyl)acetamide;
N-(4-fluorobenzyl)-2-{(1R)-5-[(methylcarbamothioyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(tetrahydr-2H-pyran-4-ylmethyl)acetamide;
N-[(1S)-1-cyclopropylethyl]-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-oxazol-4-ylmethyl)acetamide;
2-(7′-amino-1′-methyl-2,5-dioxo-2′,3′-dihydro-1H,1′H-spiro[imidazolidine-4,4′-quinolin]-1-yl)-N-[(1S)-1-cyclopropylethyl]-N-(4-fluorobenzyl)acetamide;
2-[(1R)-5-(5-cyanothiophen-2-yl)-2′,4′-dioxo-2,3-dihydro-3′H-spiro[indene-1,5-[1,3]ozazolidin]-3′-yl]-N-(4-fluorobenzyl)-N-[(2S)-1,1,1-trifluoropropan-2-yl]acetamide;
2-[(1R)-5-(4-cyano-3-fluorophenyl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(5-cyanothiophen-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1S)-1-cyclopropylethyl]-N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}acetamide;
N-[(1S)-1-cyclopropylethyl]-N-(4-fluorobenzyl)-2-{6′-[(methylcarbamoyl)amino]-2,5-dioxo-3′,4′-dihydro-1H,1′H-spiro[imidazolidine-4,2′-naphthalen]-1-yl}acetamide;
2-[6′-(acetylamino)-2,5-dioxo-3′,4′-dihydro-1H,1′H-spiro[imidazolidine-4,2′-naphthalen]-1-yl]-N-[(1S)-1-cyclopropylethyl]-N-(4-fluorobenzyl)acetamide;
2-[(1R)-5-(N″-cyano-N′-methylcarbamimidamido)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(4,6-difluoropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2 yl]acetamide;
2-{(1R)-5-[2-(acetylamino)-5-methylpyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(5-fluoropyridin-3-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(furan-3-yl)-2′,4′-dioxo-2,3-dihydro-3′H-spiro[indene-1,5′-[1,3]ozazolidin]-3′-yl]-N-[1(2S)-1,1,1-trifluoropropan-2-yl]acetamide;
2-[(1R)-2′,4′-dioxo-5-(2-oxo-2,3-dihydro-1H-benzimidazol-5-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-(4-methylpyrimidin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-2′,4′-dioxo-5-[1-(tetrahydro-2H-pyran-2-yl)-1H-pyrazol-3-yl]-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(5-fluoro-6-methylpyridin-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[6-(difluoromethyl)pyridin-3-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[6-(difluoromethyl)pyridin-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1R)-5-(furan-2-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1R)-5-[1-(ethoxymethyl)-1H-imidazol-2-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-[(1R)-5-(5-cyano-1,2-dimethyl-6-oxo-1,6-dihydropyridin-3-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
3-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]thiophene-2-carboxamide;
2-[(1R)-5-(2,6-dioxo-1,2,5,6-tetrahydropyrimidin-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-(2,4-dimethyl-1,3-thiazol-5-yl)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-{(1R)-5-[1-(cyanomethyl)-3,5-dimethyl-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
2-{(1R)-5-[3-(carbamoylamino)phenyl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-[3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)-2,2-dimethylpropyl]propanamide;
(1R)-3′-(2-{benzyl[(1S)-1-cyclopropylethyl]amino}-2-oxoethyl)-N-methyl-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(propan-2-yl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(2-methoxyethyl)-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(tetrahydrofuran-2-ylmethyl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-(tetrahydrofuran-3-ylmethyl)-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-(cyanomethyl)-2′,4′-dioxo-2,3-dihydrospiro[indene-1,5′-[1,3]ozazolidine]-5-carboxamide
(1R)-3′-(2-{benzyl[(1S)-1-cyclopropylethyl]amino}-2-oxoethyl)-N-(3-hydroxypropyl)-2′,4′-dioxo-2,3-dihydrospiro[indene-1,5′-[1,3]ozazolidine]-5-carboxamide;
3′-{2-[benzyl(1-cyclopropylethyl)amino]-2-oxoethyl}-N-(1-hydroxy-3-methylbutan-2-yl)-2′,4′-dioxo-2,3-dihydrospiro[indene-1,5′-[1,3]ozazolidine]-5-carboxamide;
N-(1-amino-4-methyl-1-oxopentan-2-yl)-3′-{2-[benzyl(1-cyclopropylethyl)amino]-2-oxoethyl}-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-[2-(diethylamino)ethyl]-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-N-[3-(1H-imidazol-1-yl)propyl]-2′,4′-dioxo-2,3-dihydrospiro[indene-1,5′-[1,3]ozazolidine]-5-carboxamide;
(1R)-3′-(2-{benzyl[(1S)-1-cyclopropylethyl]amino}-2-oxoethyl)-2′,4′-dioxo-N-[3-(2-oxopyrrolidin-1-yl)propyl]-2,3-dihydrospiro[indene-1,5′-[1,3]ozazolidine]-5-carboxamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]tetrahydropyrimidine-1(2H)-carboxamide;
3-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]tetrahydropyrimidine-1 (2H)-carboxamide;
2-(6-amino-1,1-dioxido-2′,5′-dioxo-1′H-spiro[1-benzothiophene-3,4′-imidazolidin]-1′-yl)-N-(4-fluorobenzyl)-N-[(2S)-1,1,1-trifluoropropan-2-yl]acetamide;
N-(4-fluorobenzyl)-2-(6-{1-[2-(methylamino)-2-oxoethyl]-1H-pyrazol-4-yl}-1,1-dioxido-2′,5′-dioxo-1′H-spiro[1-benzothiophene-3,4′-imidazolidin]-1′-yl)-N-[(2S)-1,1,1-trifluoropropan-2-yl]acetamide;
N-(4-fluorobenzyl)-2-{(1R)-5-[1-(2-hydroxyethyl)-1H-pyrazol-4-yl]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-phenylacetamide;
N-benzyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N,N-dibenzyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl {4-[1-(2-{(4-fluorobenzyl)[(2S)-1,1,1-trifluoropropan-2-yl]amino}-2-oxoethyl)-2,3′,5-trioxo-2′,3′-dihydrospiro[imidazolidine-4,1′-inden]-5′-yl]-1H-pyrazol-1-yl}acetate;
N-(4-fluorobenzyl)-2-{3′-fluoro-5′-[1-(2-methylpropyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide
{4-[1-(2-{(4-fluorobenzyl)[(2S)-1,1,1-trifluoropropan-2-yl]amino}-2-oxoethyl)-2,3′,5-trioxo-2′,3′-dihydrospiro[imidazolidine-4,1′-inden]-5′-yl]-1H-pyrazol-1-yl}acetic acid;
N-benzyl-N-(cyclopropylmethyl)-2 {(1R)-5-[(methylcarbamoyl)amino]-2′,4′-dioxo-2,3-dihydro-3′H-spiro[indene-1,5′-[1,3]ozazolidin]-3′-yl}acetamide;
(2R)-3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-hydroxy-2-methylpropanamide;
(2S)-3,3,3-trifluoro-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-hydroxy-2-methylpropanamide;
2-[(1R)-2′,4′-dioxo-5-({[3-(pyrrolidin-1-ylmethyl)phenyl]carbamnoyl}amino)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R)-5-({[3-(morpholin-4-ylmethyl)phenyl]carbamoyl}amino)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N³-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-N-methyl-beta-alaninamide;
N-[3-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)phenyl]propanamide;
2-(5′-{1-[2-(dimethylamino)-2-oxoethyl]-1H-pyrazol-4-yl}-2,3,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-(5′-{1-[2-(3-hydroxyazetidin-1-yl)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide;
2-(6-bromo-2′,4,4′-trioxo-3,4-dihydro-2H,3′H-spiro[naphthalene-1,5′-[1,3]ozazolidin]-3′-yl)-N-[(1S)-1-cyclopropyl-2,2,2-trifluoroethyl]-N-(4-fluorobenzyl)acetamide;
N-(4-fluorobenzyl)-2-{6-[(methylcarbamoyl)amino]-1,1-dioxido-2′,5′-dioxo-1′H-spiro[1-benzothiophene-3,4′-imidazolidin]-1′-yl}-N-[(2S)-1,1,1-trifluoropropan-2-yl]acetamide;
N-(2-chlorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)acetamide;
N-[(1S)-1-cyclopropyl-2,2,2-trifluoroethyl]-N-(4-fluorobenzyl)-2-{6-[(methylcarbamoyl)amino]-2′,4,4′-trioxo-3,4-dihydro-2H,3′H-spiro[naphthalene-1,5′-[1,3]ozazolidin]-3′-yl}acetamide;
N-(4-fluorobenzyl)-2-{(1S)-6-[(methylcarbamoyl)amino]-2′,4′-dioxo-3,4-dihydro-2H,3′H-spiro[naphthalene-1,5′-[1,3]ozazolidin]-3′-yl}-N-[(2S)-1,1,1-trifluoropropan-2-yl]acetamide;
N-(4-fluorobenzyl)-2-{(1R)-6-[(methylcarbamoyl)amino]-2′,4′-dioxo-3,4-dihydro-2H,3′H-spiro[naphthalene-1,5′-[1,3]ozazolidin]-3′-yl}-N-[(2S)-1,1,1-trifluoropropan-2-yl]acetamide;
2-[(1R)-5-(4,5-dihydro-1H-imidazol-2-ylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-(6-amino-2′,4′-dioxo-3,4-dihydro-2H,3′H-spiro[naphthalene-1,5′-[1,3]ozazolidin]-3′-yl)-N-[(1S)-1-cyclopropyl-2,2,2-trifluoroethyl]-N-(4-fluorobenzyl)acetamide;
2-[(1R)-5-({[3-(acetylamino)propyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-({[2-(acetylamino)ethyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
tert-butyl [3-({[(1R)-3′-(2-{(4-fluorobenzyl)[(2S)-1,1,1-trifluoropropan-2-yl]amino}2-oxoethyl)-2′,4′-dioxo-2,3-dihydrospiro[indene-1,5′-[1,3]ozazolidin]-5-yl]carbamoyl}amino)-2,2-dimethylpropyl]carbamate;
N-[2-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)ethyl]propanamide;
2-[(1R)-5-{[(3-amino-2,2-dimethylpropyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-5-({[3-(acetylamino)phenyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
methyl 2-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]amino}-4,5-dihydro-1H-imidazole-1-carboxylate;
2-{5-bromo-6-[(methylcarbamoyl)amino]-2′,4′-dioxo-3,4-dihydro-2H,3′H-spiro[naphthalene-1,5′-[1,3]ozazolidin]-3′-yl}-N-[(1S)-1-cyclopropyl-2,2,3-trifluoroethyl]-N-(4-fluorobenzyl)acetamide;
2-(5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-{5′-[1-(2-amino-2-oxoethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-cyclohexyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-cyclopentyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropyl-2,2,2-trifluoroethyl]-N-(4-trifluorobenzyl)-2-{4-hydroxy-6-[(methylcarbamoyl)amino]-2′,4′-dioxo-3,4-dihydro-2H,3′H-spiro[naphthalene-1,5-[1,3]ozazolidin]-3′-yl}acetamide;
N-benzyl-N-[1-(furan-2-yl)ethyl]-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[(1S)-1-cyclopropyl-2,2,2-trifluoroethyl]-N-(4-fluorobenzyl)-2-{4-fluoro-6-[(methylcarbamoyl)amino]-2′,4′-dioxo-3,4-dihydro-2H,3′H-spiro[naphthalene-1,5′-[1,3]ozazolidin]-3′-yl}acetamide;
2-{5′-[1-(2-amino-2-oxoethyl)-1H-pyrazol-4-yl]-3′-hydroxy-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-{[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-beta-alanine;
N-cyclopropyl-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl N-{[(1R)-3-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}-beta-alaninate;
N-(3,5-difluorobenzyl)-2-{(1R)-5-[(methylcarbamoyl)amino]-2′,4′-dioxo-2,3-dihydro-3′I-spiro[indene-1,5′-[1,3]ozazolidin]-3′-yl}-N-[(2S)-1,1,1-trifluoropropan-2-yl]acetamide;
N-(4-fluorobenzyl)-N-(3-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(1S)-5-amino-2′,4′-dioxo-2,3-dihydro-3′-spiro[indene-1,5′-[1,3]ozazolidin]-3′-yl]-N-[(1R)-1-cyclopropylethyl]-N-(4-fluorobenzyl)acetamide;
2-[(1R)-5-amino-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
ethyl ({(3R)-3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(3R)-piperidin-3-yl]acetamide;
ethyl ({(3R)-3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]piperidin-1-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(methylsulfonyl)piperidin-4-yl]acetamide;
2-[(4S)-5′-{1-[2-(dimethylamino)-2-oxoethyl]-1H-pyrazol-4-yl}-2,3′,5′-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4S)-5′-{1-[2-(dimethylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-N-[(2S))-1,1,1-trifluoropropan-2-yl]-2-[(4R)-2,3′,5-trioxo-2′,3′-dihydro-1H-spiro[imidazolidine-4,1′-inden]-1-yl]acetamide;
N-(4-fluorobenzyl)-N-[(2S)-1,1,1-trifluoropropan-2-yl]-2-[(4S)-2,3′,5-trioxo-2′,3′-dihydro-1H-spiro[imidazolidine-4,1′-inden]-1-yl]acetamide;
N-(4-fluorobenzyl)-2-[5′-(1-methyl-1H-pyrazol-4-yl)-2,3′,5-trioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-{6-fluoro-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(4-fluorobenzyl)-2-[3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(5-amino-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
2-(7′-bromo-1′-methyl-2,5-dioxo-2′,3′-dihydro-1H,1′H-spiro[imidazolidine-4,4′-quinolin]-1-yl)-N-[(1S)-1-cyclopropyl-2,2,2-trifluoroethyl]-N-(4-fluorobenzyl)acetamide;
N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1-phenylethyl)acetamide;
2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)-N-[2-(trifluoromethyl)benzyl]acetamide;
2-(5-bromo-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}-N-[(2S)-1,1,1-trifluoropropan-2-yl]acetamide;
N-(4-fluorobenzyl)-2-[(3′R,4R)-3′-hydroxy-2,5-dioxo-2′,3′-dihydro-1H-spiro[imidazolidine-4,1′-inden]-1-yl]-N-[(2S)-1,1,1-trifluoropropan-2-yl]acetamide;
N-benzyl-N-(cyanomethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-N-(2-cyanoethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(methylsulfonyl)propan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(1-methyl-1H-pyrazol-4-yl)methyl]acetamide;
N-[(1S)-1-cyclopropylethyl]-N-(4-fluorobenzyl)-2-{4-methoxy-5-[(methylcarbamoyl)amino]-2′,4′-dioxo-2,3-dihydro-3′H-spiro[indene-1,5′-[1,3]ozazolidin]-3′-yl}acetamide;
2-[5-(acetylamino)-4-methoxy-2′,4′-dioxo-2,3-dihydro-3′H-spiro[indene-1,5′-[1,3]ozazolidin]-3′-yl]-N-[(1S)-1-cyclopropylethyl]-N-(4-fluorobenzyl)acetamide;
N-(4-fluorobenzyl)-2-[(3′S,4R)-3′-fluoro-2,5-dioxo-2′,3′-dihydro-1H-spiro[imidazolidine-4,1′-inden]-1-yl]-N-[(2S)-1,1,1-trifluoropropan-2-yl]acetamide;
2-(7′-bromo-2,2′,5-trioxo-2′,3′-dihydro-1H,1′H-spiro[imidazolidine-4,4′-quinolin]-1-yl)-N-(4-fluorobenzyl)-N-[(2S)-1,1,1-trifluoropropan-2-yl]acetamide;
2-[(1R)-5-({[3-(acetylamino)-2,2-dimethylpropyl]carbamoyl}amino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-(7′-{1-[2-(methylamino)-2-oxoethyl]-1H-pyrazol-4-yl}-2,2′,5-trioxo-2′,3′-dihydro-1H,1′H-spiro[imidazolidine-4,4′-quinolin]-1-yl)-N-[(2S)-1,1,1-trifluoropropan-2-yl]acetamide;
2-{7′-[1-(2-amino-2-oxoethyl)-1H-pyrazol-4-yl]-2,2′,5-trioxo-2′,3′-dihydro-1H,1′H-spiro[imidazolidine-4,4′-quinolin]-1-yl}-N-(4-fluorobenzyl)-N-[(2S)-1,1,1-trifluoropropan-2-yl]acetamide;
N-cyclopentyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-[(1S)-1-cyclopropyl-2,2,2-trifluoroethyl]-N-(4-fluorobenzyl)-2-{1′-methyl-7′-[(methylcarbamoyl)amino]-2,5-dioxo-2′,3′-dihydro-1H,1′H-spiro[imidazolidine-4,4′-quinolin]-1-yl}acetamide;
2-(5′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2,2,2-trifluoro-N-[1-(2-{(4-fluorobenzyl)[(2S)-1,1,1-trifluoropropan-2-yl]amino}-2-oxoethyl)-1′-methyl-2,5-dioxo-2′,3′-dihydro-1′H-spiro[imidazolidine-4,4′-quinolin]-7′-yl]acetamide;
N-[1-(2-{[(1S)-1-cyclopropylethyl](4-fluorobenzyl)amino}-2-oxoethyl)-1′-methyl-2,5-dioxo-2′,3′-dihydro-1′H-spiro[imidazolidine-4,4′-quinolin]-7′-yl]-2,2,2-trifluoroacetamide;
N-(4-fluorobenzyl)-2-[(3′S,4S)-3′-hydroxy-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(pyridin-3-yl)acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1-methyl-1H-pyrazol-4-yl)acetamide;
N-(4-fluorobenzyl)-2-[3′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-benzyl-N-(2,2-difluorocyclopentyl)-2-{5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-(2-methylcyclopropyl)acetamide;
N-(2,2-dimethylcyclopentyl)-N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{5-[(methylcarbamoyl)amino]-2′,4′-dioxo-2,3-dihydro-3′H-spiro[indene-1,5′-[1,3]ozazolidin]-3′-yl}-N-(2-methylcyclopentyl)acetamide;
2-(5′-bromo-3′-hydroxy-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-(5-{[(2-cyanoethyl)carbamoyl]amino}-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
N-(4-fluorobenzyl)-2-[(3R′,4S)-3′-fluoro-2,5-dioxo-2′,3′-dihydro-1H-spiro[imidazolidine-4,1′-inden]-1-yl]-N-[(2S)-1,1,1-trifluoropropan-2-yl]acetamide;
methyl [(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamate;
4-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)butanoic acid;
N²-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylalaninamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-2-methylalaninamide;
N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide;
N-cyclobutyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
N-cyclopentyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(1,3-oxazol-4-ylmethyl)acetamide;
N-benzyl-2-{(1R)-5-[(methylcarbamoyl)amino]-2′,4′-dioxo-2,3-dihydro-3′H-spiro[indene-1,5′ [1,3]ozazolidin]-3′-yl}-N-[1-(pyridin-2-yl)ethyl]acetamide;
N-(4-fluorobenzyl)-2-{5′-[((methylcarbamoyl))amino]-2,3′,5-trioxo-2′,3′-dihydro-1H-spiro[imidazolidine-4,1′-inden]-1-yl}-N-[(2S)-1,1,1-trifluoropropan-2-yl]acetamide;
N-(4-chlorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(3,5-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-(5-{[(cyanomethyl)carbamoyl]amino}-6-fluoro-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
2-[(1R)-5-{[(cyanomethyl)carbamoyl]amino}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclohexyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1R)-1-phenylethyl]acetamide;
2-(5′-amino-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-pyrrolidin-3-yl]acetamide;
N-(4-fluorobenzyl)-N-(trans-3-hydroxycyclobutyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-1-(methylsulfonyl)pyrrolidin-3-yl]acetamide;
ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]pyrrolidin-1-yl}sulfonyl)carbamate;
N-(4-fluorobenzyl)-2-[6-(1-methyl-1H-pyrazol-4-yl)-1,1-dioxido-2′,5′-dioxo-1′H1-spiro[1-benzothiophene-3,4′-imidazolidin]-1′-yl]-N-[(2S)-1,1,1-trifluoropropan-2-yl]acetamide;
2-[(4S)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{3′-hydroxy-5′-[1-(2-methylpropyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide
N-(4-fluorobenzyl)-2-[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
N-(1-cyanopropan-2-yl)-N-(4-fluorobenzyl)-2-{(1)-5-[(methylcarbamoyl)amino]-2′,4′-dioxo-2,3-dihydro-3′H-spiro[indene-1,5′-[1,3]ozazolidin]-3′-yl}acetamide;
2-[(4S)-5′-bromo-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-(5′-bromo-3′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
5-({[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]carbamoyl}amino)pentanoic acid;
N-[(1S)-1-cyclopropyl-2,2,2-trifluoroethyl]-N-(4-fluorobenzyl)-2-[4-fluoro-5-(1-methyl-1H-pyrazol-4-yl)-2′,4′-dioxo-2,3-dihydro-3′H-spiro[indene-1,5′-[1,3]ozazolidin]-3′-yl]acetamide;
N-(4-fluorobenzyl)-2-[6-fluoro-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(4S)-5′-[1-(2-amino-2-oxoethyl)-1H-pyrazol-4-yl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-[(1S)-1-cyclopropylethyl]-N-(4-fluorobenzyl)-2-[1′-methyl-7′-(1-methyl-1H-pyrazol-4-yl)-2,5-dioxo-2′,3′-dihydro-1H,1′H-spiro[imidazolidine-4,4′-quinolin]-1-yl]acetamide;
N-(4-fluorobenzyl)-2-[7′-(1-methyl-1H-pyrazol-4-yl)-2,2′,5-trioxo-2′,3′-dihydro-1H,1′H-spiro[imidazolidine-4,4′-quinolin]-1-yl]-N-[(2S)-1,1,1-trifluoropropan-2-yl]acetamide;
N-(4-fluorobenzyl)-2-[(4S)-5′-(1-methyl-1H-pyrazol-4-yl)-2,3′,5-trioxo-2′,3′-dihydro-1H-spiro[imidazolidine-4,1′-inden]-1-yl]-N-[(2S)-1,1,1-trifluoropropan-2-yl]acetamide;
N-[(3R)-1-acetylpyrrolidin-3-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R) 1-(2-sulfamoylethyl)pyrrolidin-3-yl]acetamide;
N-(4-fluorobenzyl)-N-(trans-4-hydroxycyclohexyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
methyl (1R,3S)-3-[(4-fluorobenzyl)({(1R)-5-[(methylcarbamoyl)amino]-2′,4′-dioxo-2,3-dihydro-3′H-spiro[indene-1,5-[1,3]ozazolidin]-3′-yl}acetyl)amino]cyclohexanecarboxylate;
N-(4-fluorobenzyl)-2-[(3′R,4S)-3′-hydroxy 2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(3′S,4S)-3′-hydroxy-5′-(1-methyl-1H-pyrazol-4-yl)-2,5-dioxo-2,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(4S)-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl]-N-[(2S)-1,1,1-trifluoropropan-2-yl]acetamide;
N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]-3-(methylsulfonyl)tetrahydropyrimidine-1(2H)-carboxamide;
N-(4-fluorobenzyl)-2-[(3′S,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(4S)-5′ {1-[2-(methylamino)-2-oxoethyl]-1H-pyrazol-4-yl}-2,3′,5-trioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(3′S,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(3′R,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
N²-acetyl-N-[(1R)-3′-(2-{(4-fluorobenzyl)[(2S)-1,1,1-trifluoropropan-2-yl]amino}-2-oxoethyl)-2′,4′-dioxo-2,3-dihydrospiro[indene-1,5′-[1,3]ozazolidin]-5-yl]glycinamide;
2-{(1R)-5-[(methylcarbamoyl)amino]-2′,4′-dioxo-2,3-dihydro-3′H-spiro[idene-1,5′-[1,3]ozazolidin]-3′-yl}-N-[1-(2-methylphenyl)ethyl]-N-(2-methylpropyl)acetamide;
N-(2-chlorobenzyl)-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(2-methylpropyl)acetamide;
2-[(3′S,4S)-5′-bromo-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-N-(4-methoxyphenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(4-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-(2-fluorophenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-[(3R,4S)-3′-fluoro-5′-{-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(3′R,4S)-5′-bromo-3′-hydroxy-2,5-dioxo-2′,3′-dihydro-H-spiro[imidazolidine-4,1′-inden]-1-yl]-N-(4-fluorobenzyl)-N-[(2S)-1,1,1-trifluoropropan-2-yl]acetamide;
2-{(4S)-5′-[1-(2-amino-2-oxoethyl)-1H-pyrazol-4-yl]-2,3′,5′-trioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[1-(trifluoromethyl)cyclopropropyl]acetamide
2-[(1R)-5-(3,4-dihydro-2H-pyrrol-5-ylamino)-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(3,4-difluorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropropan-2-yl]acetamide;
N-(3-fluorobenzyl)-2-{5-[(methylcarbamoyl))amino]-2′,4′-dioxo-2,3-dihydro-3′H-spiro[idene-1,5′-[1,3]ozazolidin]-3′-yl}-N-[(2S)-1,1,1-trifluoropropan-2-yl]acetamide;
N-(2,5-difluorobenzyl)-2-{5-[(methylcarbamoyl)amino]-2′,4′-dioxo-2′,3′-dihydro-3′H-spiro[indene-1,5′-[1,3]ozazolidin]-3′-yl}-N-[(2S)-1,1,1-trifluoropropan-2-yl]acetamide;
N-(3-chlorobenzyl)-2-{5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
2-{(3′S,4 S)-5′-[1-(2-amino-2-oxoethyl)-1H-pyrazol-4-yl]-3′-hydroxy-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-{5-[1-(2-amino-2-oxoethyl)-1H-pyrazol-4-yl]-6-fluoro-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
2-[(1R)-5-bromo-2′,4′-dioxo-2,3-dihydro-3′H-spiro[indene-1,5′-[1,3]ozazolidin]-3′-yl]-N-(4-fluorobenzyl)-N-[(3R)-pyrrolidin-3-yl]acetamide;
ethyl ({(3R)-3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]pyrrolidin-1-yl}sulfonyl)carbamate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(piperidin-4-yl)acetamide;
ethyl ({4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
2-[(3′S,4S)-5′-{1-[2-(dimethylamino)-2-oxoethyl]-1H-pyrazol-4-yl}-3′-hydroxy-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]azetidine-1-carboxylate;
N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}-N-[2-(trifluoromethyl)benzyl]acetamide;
N-(2-chloro-4-fluorobenzyl)-N-(cyclopropylmethyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(3′R,4S)-5′-{1-[2-(dimethylamino)-2-oxoethyl]-11H-pyrazol-4-yl}-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-N-(3-methoxyphenyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-benzyl-2-{(1R)-5-[(methylcarbamoyl)amino]-2′,4′-dioxo-2,3-dihydro-3′H-spiro[indene-1,5-[1,3]ozazolidin]-3′-yl}-N-(pyridin-4-yl)acetamide
N-(4-fluorobenzyl)-2-[(4S)-5′-{1-[2-(methylamino)-2-oxoethyl]-1H-pyrazol-4-yl}-2,5-dioxo-1H-spiro[imidazolidine-4,1′-inden]-1-yl]-N-[(2S)-1,1,1-trifluoropropan-2-yl]acetamide;
N-(3,4-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1-trifluoropropan-2-yl]acetamide;
N-(4-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(3-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(2,5-difluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide
N-(3-chlorobenzyl)-2-[(4S)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-[(1S)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-[(1S)-1-cyclopropyl-2,2,2-trifluoroethyl]-N-(4-fluorobenzyl)-2-[(4S)-5′-(1-methyl-H-pyrazol-4-yl)-2,5-dioxo-2′,3′-dihydro-1H-spiro[imidazolidine-4,1′-inden]-1-yl]acetamide;
N-[(1R)-1-cyclopropylethyl]-N-(4-fluorobenzyl)-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-benzyl-N-[(1S)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide;
N-benzyl-N-[(1R)-1-cyclopropylethyl]-2-[(4S)-5′-(1-methyl-1H-pyrazol-4-yl)-2,5-dioxo-2′,3′-dihydro-1H-spiro[imidazolidine-4,1′-inden]-1-yl]acetamide;
2-(4′-bromo-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[4′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-(6-fluoro-5-{1-[2-(methylamino)-2-oxoethyl]-1H-pyrazol-4-yl}-2′,4′-dioxo-2,3-dihydro-3′-spiro[indene-1,5′-[1,3]ozazolidin]-3′-yl)-N-[(2S)-1,1,1-trifluoropropan-2-yl]acetamide;
2-[(3′S,4S)-5′-{1-[2-(dimethylamino)-2-oxoethyl]-1H-pyrazol-4-yl}-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
methyl (1S,3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(tetrahydro-2H-pyran-4-yl)acetamide;
N-(1,1-dioxidotetrahydro-2H-thiopyran-4-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(2,2-difluorocyclopentyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-cyclohexyl-2-{(1R)-5-[(methylcarbamoyl)amino]-2′,4′-dioxo-2,3-dihydro-3′H-spiro[indene-1,5′-[1,3]ozazolidin]-3′-yl}-N-[(1S)-1-phenylethyl]acetamide; and
2-[(3S)-6-amino-1,1-dioxido-2′,5′-dioxo-1′H-spiro[1-benzothiophene-3,4′-imidazolidin]-1′-yl]-N-(4-fluorobenzyl)-N-[(2S)-1,1,1-trifluoropropan-2-yl]acetamide;
2-[(3′R,4S)-5′-bromo-3′-fluoro-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-(5′-bromo-6′-fluoro-2,3′,5-trioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
2-(5′-bromo-6′-fluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-(6′-fluoro-5′-{1-[2-(methylamino)-2-oxoethyl]-1H-pyrazol-4-yl}-2,3′,5-trioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-(6′-fluoro-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
N-(4-fluorobenzyl)-2-[4′-(1-methyl-1,2,3,6-tetrahydropyridin-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-[(1S)-1-cyclopropyl-2,2,2-trifluoroethyl]-N-(4-fluorobenzyl)-2-[7-(1-methyl-1H-pyrazol-4-yl)-2′,4′-dioxo-1H,3′H-spiro[isochromene-4,5′-[1,3]ozazolidin]-3′-yl]acetamide;
2-(7-amino-2′,4′-dioxo-1H,3′H-spiro[isochromene-4,5′-[1,3]ozazolidin]-3′-yl)-N-[(1S)-1-cyclopropyl-2,2,2-trifluoroethyl]-N-(4-fluorobenzyl)acetamide;
N-[(1S)-1-cyclopropyl-2,2,2-trifluoroethyl]-N-(4-fluorobenzyl)-2-{7-[(methylcarbamoyl)amino]-2′,4′-dioxo-1H,3′H-spiro[isochromene-4,5′-[1,3]ozazolidin]-3′-yl}acetamide;
N-(4-fluorobenzyl)-2-{7-[(methylcarbamoyl)amino]-2′,4′-dioxo-1H,3′H-spiro[isochromene-4,5′-[1,3]ozazolidin]-3′-yl}-N-[(2S)-1,1,1-trifluoropropan-2-yl]acetamide;
N-(4-fluorobenzyl)-2-[7-(1-methyl-1H-pyrazol-4-yl)-2′,4′-dioxo-1H,3′H-spiro[isochromene-4,5′-[1,3]ozazolidin]-3′-yl]-N-[(2S)-1,1,1-trifluoropropan-2-yl]acetamide;
N-(4-fluorobenzyl)-2-(7-{1-[2-(methylamino)-2-oxoethyl]-1H-pyrazol-4-yl}-2′,4′-dioxo-1H,3′H-spiro[isochromene-4,5′-[1,3]ozazolidin]-3′-yl)-N-[(2S)-1,1,1-trifluoropropan-2-yl]acetamide;
N-(4-fluorobenzyl)-2-[(3′R,4S)-3′-hydroxy-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
2-[(3R)-6-amino-1,1-dioxido-2′,5′-dioxo-1′H-spiro[1-benzothiophene-3,4′-imidazolidin]-1′-yl]-N-(4-fluorobenzyl)-N-[(2S)-1,1,1-trifluoropropan-2-yl]acetamide;
N-(4-fluorobenzyl)-N-{1-[2-(methylamino)-2-oxoethyl]-1H-pyrazol-4-yl}-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[1-(cyanomethyl)-1H-pyrazol-4-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
tert-butyl 3-{1-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]ethyl}azetidine-1-carboxylate;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxylic acid;
ethyl ({(3S)-3-[(4-fluorobenzyl) {[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
ethyl ({(3S)-3-[{[(1R)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}(4-fluorobenzyl)amino]pyrrolidin-1-yl}sulfonyl)carbamate;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}acetate;
ethyl ({(3R)-3-[(4-fluorobenzyl) {[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]piperidin-1-yl}sulfonyl)carbamate;
ethyl ({(3R)-3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]piperidin-1-yl}sulfonyl)carbamate;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}acetic acid;
2-{2,5-dioxo-5′-[(2,2,2-trifluoroethyl)amino]-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-[(4S)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(4R)-5′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{(3′R,4S)-5′-[1-(2-amino-2-oxoethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-(5′-bromo-6′-fluoro-3′-hydroxy-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-(6′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl)-N-[(2S)-1,1,1-trifluoropropan-2-yl]acetamide;
2-{5′-[1-(2-amino-2-oxoethyl)-1H-pyrazol-4-yl]-6′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-{6′-fluoro-5′-[1-(2-hydroxyethyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-[6′-fluoro-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-(5′-bromo-3′,6′-difluoro-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
tert-butyl 4-[1-(2-{(4-fluorobenzyl) [(2S)-1,1,1-trifluoropropan-2-yl]amino}-2-oxoethyl)-2,5-dioxo-2′,3′-dihydrospiro[imidazolidine-4,1′-inden]-4′-yl]-3,6-dihydropyridine-1(2H)-carboxylate;
N-(4-fluorobenzyl)-2-{(4S)-7-[(methylcarbamoyl)amino]-2′,4′-dioxo-1H,3′H-spiro[isochromene-4,5′-[1,3]ozazolidin]-3′-yl}-N-[(2S)-1,1,1-trifluoropropan-2-yl]acetamide;
2-[2,5-dioxo-4′-(1,2,3,6-tetrahydropyridin-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{(4R)-7-[(methylcarbamoyl)amino]-2′,4′-dioxo-1H,3′H-spiro[isochromene-4,5′-[1,3]ozazolidin]-3′-yl}-N-[(2S)-1,1,1-trifluoropropan-2-yl]acetamide;
2-{5′-[1-(2-amino-2-oxoethyl)-1H-pyrazol-4-yl]-3′,6′-difluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-{4-[3′,6′-difluoro-1-(2-{(4-fluorobenzyl) [(2S)-1,1,1-trifluoropropan-2-yl]amino}-2-oxoethyl)-2,5-dioxo-2′,3′-dihydro-1H-spiro[imidazolidine-4,1′-inden]-5′-yl]-1H-pyrazol-1-yl}-N,N-dimethylacetamide;
2-[3′,6′-difluoro-5′-(1-methyl-1H-pyrazol-4-yl)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{3′,6′-difluoro-5′-[1-(2-hydroxyethyl)-1H-pyrazol-4-yl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-[(4R)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
tert-butyl (3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
tert-butyl (3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3R)-piperidin-3-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(3S)-piperidin-3-yl]acetamide;
N-[4-amino-3-(hydroxymethyl)butan-2-yl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({(3S)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzamide;
tert-butyl 2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidine-1-carboxylate;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(pyrrolidin-2-ylmethyl)acetamide;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexylidene}acetate;
N-ethyl-4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexanecarboxamide;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexylidene}acetic acid;
2-[(4S)-2,5-dioxo-5′-(1H-pyrazol-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{5′-[(1-methyl-1H-pyrazol-3-yl)amino]-2,5-dioxo-2′,3′-dihydro-1H-spiro[imidazolidine-4,1′-inden]-1-yl}-N-[(2S)-1,1,1-trifluoropropan-2-yl]acetamide;
2-[(4S)-5′-amino-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{4′-[(dimethylamino)methyl]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-[4′-(morpholin-4-ylmethyl)-2,5-dioxo-2′,3′-dihydro-1H-spiro[imidazolidine-4,1′-inden]-1-yl]-N-[(2S)-1,1,1-trifluoropropan-2-yl]acetamide;
2-[2,5-dioxo-4′-(pyrrolidin-1-ylmethyl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-(4′-amino-2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-[(1R)-5-(oxetan-3-ylamino)-2′,4′-dioxo-2,3-dihydro-3′H-spiro[indene-,5′-[1,3]ozazolidin]-3′-yl]-N-[(2S)-1,1,1-trifluoropropan-2-yl]acetamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzamide;
N-[(1-acetylpyrrolidin-2-yl)methyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{[1-(methylsulfonyl)pyrrolidin-2-yl]methyl}acetamide;
2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}-N-methylpyrrolidine-1-carboxamide;
N-(3,4-difluorobenzyl)-2-[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{[1-(methylsulfamoyl)pyrrolidin-2-yl]methyl}acetamide;
N-(cyclopropylmethyl)-N-(2,3-dihydro-H-inden-1-yl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
methyl trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylate;
trans-4-{[(5′-bromo-2,5-dioxo-2′,3′-dihydro-1H-spiro[imidazolidine-4,1′-inden]-1-yl)acetyl](4-fluorobenzyl)amino}cyclohexanecarboxylic acid;
trans-4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]cyclohexanecarboxylic acid;
2-[(4R)-7′-bromo-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
trans-4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}cyclohexanecarboxylic acid;
2-[(4S)-7′-bromo-2,5-dioxo-2′,3′-dihydro-H-spiro[imidazolidinne-4,1′-inden]-1-yl]-N-(4-fluorobenzyl)-N-[(2S)-1,1,1-trifluoropropan-2-yl]acetamide;
ethyl ({4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]piperidin-1-yl}sulfonyl)carbamate;
ethyl ({(3R)-3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]piperidin-1-yl}sulfonyl)carbamate;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexyl}acetic acid;
2-[(4S)-5′-{[(2-cyanoethyl)carbamoyl]amino}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-{(4S)-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(4S)-5′-[(methylcarbamoyl)amino]-2,3′,5-trioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(3′S,4S)-3′-hydroxy-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
N-(4-fluorobenzyl)-2-{(3′R,4S)-3′-fluoro-5′-[1-(2-hydroxyethyl)-1H-pyrazol-4-yl]-2,5-dioxo-2′,3′-dihydro-1H-spiro[imidazolidine-4,1′-inden]-1-yl}-N-[(2S)-1,1,1-trifluoropropan-2-yl]acetamide;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-3-methylazetidin-1-yl}sulfonyl)carbamate;
2-[4′-(acetylamino)-2,5-dioxo-2′,3′-dihydro-1H-spiro[imidazolidine-4, 1′-inden]-1-yl]-N-(4-fluorobenzyl)-N-[(2S)-1,1,1-trifluoropropan-2-yl]acetamide;
2-[(4R)-5′-bromo-6′-fluoro-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(3-cyanophenyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(3′R,4S)-3′-fluoro-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
N-(3,4-difluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
N-(4-fluorobenzyl)-2-{7′-[(methylcarbamoyl)amino]-2,2′,5-trioxo-2′,3′-dihydro-1H, 1′H-spiro[imidazolidine-4,4′-quinolin]-1-yl}-N-[(2S)-1,1,1-trifluoropropan-2-yl]acetamide;
tert-butyl (4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetate;
(4-{[(5′-bromo-2,5-dioxo-2′,3′-dihydro-1H-spiro[imidazolidine-4,1′-inden]-1-yl)acetyl](4-fluorobenzyl)amino}cyclohexylidene)acetic acid;
{4-[(4-fluorobenzyl) {[5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
(4-{(4-fluorobenzyl) [(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}cyclohexylidene)acetic acid;
tert-butyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}carbamate;
N-(3-aminocyclobutyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-[3-(acetylamino)cyclobutyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{3-[(methylsulfonyl)amino]cyclobutyl}acetamide;
N-(4-fluorobenzyl)-2-[(3′R,4S)-3′-hydroxy-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2-[(4R)-5′-(acetylamino)-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
N-(4-fluorobenzyl)-2-[(3′E,4S)-3′-(hydroxyimino)-5′-(1-methyl-1H-pyrazol-4-yl)-2,5-dioxo-2′,3′-dihydro-1H-spiro[imidazolidine-4,1′-inden]-1-yl]-N-[(2S)-1,1,1-trifluoropropan-2-yl]acetamide;
2,2′-({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}imino)diacetic acid;
N-{3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}glycine;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxylate;
methyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}carbamate;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutyl}sulfamoyl)carbamate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxylic acid;
2-[(3′R,4S)-3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{4-[(3′R,4S)-3′,6′-difluoro-1-[(2-{(4-fluorobenzyl) [(2S)-1,1,1-trifluoropropan-2-yl]amino}-2-oxoethyl)-2,5-dioxo-2′,3′-dihydro-1H-spiro imidazolidine-4,1′-inden]-5′-yl]-1H-pyrazol-1-yl}-N,N-dimethylacetamide;
2-[(3′S,4R)-3′,6′-difluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-{4-[(3′S,4R)-3′,6′-difluoro-1-(2-{(4-fluorobenzyl)[(2S)-1,1,1-trifluoropropan-2-yl]amino}-2-oxoethyl)-2,5-dioxo-2′,3′-dihydro-1H-spiro[imidazolidine-4,1′-inden]-5′-yl]-1H-pyrazol-1-yl}-N,N-dimethylacetamide;
ethyl [(4-{(4-fluorobenzyl)[(5′-{1-[2-(methylamino)-2-oxoethyl]-1H-pyrazol-4-yl}-2,5-dioxo-2′,3′-dihydro-1H-spiro[imidazolidine-4,1′-inden]-1-yl)acetyl]amino}piperidin-1-yl)sulfonyl]carbamate;
ethyl ({4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]piperidin-1-yl}sulfonyl)carbamate;
2-[(1 S,3S)-5-bromo-3-hydroxy-2′,4′-dioxo-2,3-dihydro-3′H-spiro[indene-1,5′-[1,3]ozazolidin]-3′-yl]-N-(4-fluorobenzyl)-N-[(2S)-1,1,1,1-trifluoropropan-2-yl]acetamide;
2-(5-chloro-4-cyano-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
benzyl 3-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}azetidine-1-carboxylate;
tert-butyl 4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoate;
tert-butyl 4-[{[(4S)-5′-bromo-2,5-dioxo-2′,3′-dihydro-1H-spiro[imidazolidine-4,1′-inden]-1-yl]acetyl}(4-fluorobenzyl)amino]piperidine-1-carboxylate;
N-(4-fluorobenzyl)-2-[(1 S,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
ethyl ({4-[{[(4S)-5′-amino-2,5-dioxo-2′,3′-dihydro-1H-spiro[imidazolidine-4,1′-inden]-1-yl]acetyl}(4-fluorobenzyl)amino]piperidin-1-yl}sulfonyl)carbamate;
ethyl ({3,3-difluoro-4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]pyrrolidin-1-yl}sulfonyl)carbamate;
ethyl ({3,3-difluoro-4-[(4-fluorobenzyl) {[(4S)-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]acetyl}amino]pyrrolidin-1-yl}sulfonyl)carbamate;
ethyl ({3,3-difluoro-4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
ethyl ({3,3-difluoro-4-[(4-fluorobenzyl){[(1R)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]acetyl}amino]pyrrolidin-1-yl}sulfonyl)carbamate;
N-(4-fluorobenzyl)-2-[(1S)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1S)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-N-(3-methoxycyclobutyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
3-[(4-fluorobenzyl)({(1R)-5-[(methylcarbamoyl)amino]-2′,4′-dioxo-2,3-dihydro-3′H-spiro[indene-,5′-[1,3]ozazolidin]-3′-yl}acetyl)amino]-N-methylcyclobutanecarboxamide;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclobutanecarboxamide;
2-{(3′R,4S)-5′-[1-(difluoromethyl)-1H-pyrazol-4-yl]-3′-fluoro-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
2-(5-amino-4-cyano-2′,4′-dioxo-2,3-dihydro-3′H-spiro[indene-1,5′-[1,3]ozazolidin]-3′-yl)-N-(4-fluorobenzyl)-N-[(2S)-1,1,1-trifluoropropan-2-yl]acetamide;
2-[2,5-dioxo-4′-(piperidin-4-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
tert-butyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoate;
4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
tert-butyl {4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
{4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetic acid;
(2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidin-1-yl)acetic acid;
methyl {3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetate;
N-(4-fluorobenzyl)-2-[(4S)-7′-hydroxy-2,5-dioxo-2′,3′-dihydro-1H-spiro[imidazolidine-4, 1′-inden]-1-yl]-N-[(2S)-1,1,1-trifluoropropan-2-yl]acetamide;
{3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]phenyl}acetic acid;
tert-butyl {4-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]cyclohexylidene}acetate;
tert-butyl {4-[{[(4S)-5′-bromo-2,5-dioxo-2′,3′-dihydro-1H-spiro[imidazolidine-4,1′-inden]-1-yl]acetyl}(4-fluorobenzyl)amino]cyclohexylidene}acetate;
tert-butyl {4-[(4-fluorobenzyl) {[(1S)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetate;
tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate;
{4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetic acid;
{4-[{[(4S)-5′-amino-2,5-dioxo-2′,3′-dihydro-1H-spiro[imidazolidine-4,1′-inden]-1-yl]acetyl}(4-fluorobenzyl)amino]cyclohexylidene}acetic acid;
N-(4-fluorobenzyl)-2-[(1 S,3R)-3-fluoro-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(4S)-5′-bromo-6′-fluoro-2,3′,5-trioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
2-[(3′R,4R)-5′-bromo-6′-fluoro-3′-hydroxy-2,5-dioxo-2′,3′-dihydro-1H-spiro[imidazolidine-4, 1′-inden]-1-yl]-N-(4-fluorobenzyl)-N-[(2S)-1,1,1-trifluoropropan-2-yl]acetamide;
2-[(3′S,4S)-5′-bromo-6′-fluoro-3′-hydroxy-2,5-dioxo-2′,3′-dihydro-1H-spiro[imidazolidine-4, 1′-inden]-1-yl]-N-(4-fluorobenzyl)-N-[(2S)-1,1,1-trifluoropropan-2-yl]acetamide;
N-(4-fluorobenzyl)-2-[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]-N-(tetrahydro-2H-pyran-4-yl)acetamide;
2-(2,5-dioxo-2′,3′-dihydro-1H-spiro[imidazolidine-4,1′-inden]-1-yl)-N-(4-fluorobenzyl)-N-[(2S)-1,1,1-trifluoropropan-2-yl]acetamide;
N-(4-fluorobenzyl)-2-{6′-fluoro-5′-[(methylcarbamoyl)amino]-2,5-dioxo-2′,3′-dihydro-1H-spiro[imidazolidine-4,1′-inden]-1-yl}-N-[(2S)-1,1,1-trifluoropropan-2-yl]acetamide;
2-{(3′R,4S)-3′,6′-difluoro-5′-[(methylcarbamoyl)amino]-2,5-dioxo-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(azetidin-3-ylmethyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
{4-[{[(1R)-2′,4′-dioxo-2,3-dihydro-3′H-spiro[indene-1,5′-[1,3]ozazolidin]-3′-yl]acetyl}(4-fluorobenzyl)amino]cyclohexylidene}acetic acid;
methyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetate;
{4-[(4-fluorobenzyl) {[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
tert-butyl {trans-4-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]cyclohexyl}carbamate;
N-(trans-4-aminocyclohexyl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-{trans-4-[(methylsulfonyl)amino]cyclohexyl}acetamide;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(trans-4-{[(trifluoromethyl)sulfonyl]amino}cyclohexyl)acetamide;
N-(4-fluorobenzyl)-2-[(4R)-7′-hydroxy-2,5-dioxo-2′,3′-dihydro-H-spiro[imidazdaolidine-4,1′-inden]-1-yl]-N-[(2S)-1,1,1-trifluoropropan-2-yl]acetamide;
tert-butyl {4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetate;
{4-[(4-fluorobenzyl) {[(4S)-5′-(1-methyl-1H-pyrazol-4-yl)-2,5-dioxo-2′,3′-dihydro-1H-spiro[imidazolidine-4,1′-inden]-1-yl]acetyl}amino]cyclohexyl}acetic acid;
tert-butyl 6-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-2-azaspiro[3.3]heptane-2-carboxylate;
N-(2-azaspiro[3.3]hept-6-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
2-[(1 S,3R)-5-bromo-3-hydroxy-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-[(1-acetylazetidin-3-yl)methyl]-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
N-(4-fluorobenzyl)-N-[(2S)-1,1,1-trifluoropropan-2-yl]-2-(2′,3,4′-trioxo-1,2,3,4,8,9-hexahydro-3′H-spiro[cyclopenta[f]quinazoline-7,5′-[1,3]ozazolidin]-3′-yl)acetamide;
ethyl [(3-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}azetidin-1-yl)sulfonyl]carbamate;
ethyl [(2-{[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]methyl}pyrrolidin-1-yl)sulfonyl]carbamate;
2-[(4S)-2,5-dioxo-5′-(1H-pyrazol-5-yl)-2′,3′-dihydro-1H-spiro[imidazolidine-4,1′-inden]-1-yl]-N-(4-fluorobenzyl)-N-[(2S)-1,1,1-trifluoropropan-2-yl]acetamide;
ethyl ({6-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]-2-azaspiro[3.3]hept-2-yl}sulfonyl)carbamate;
4-[(4-fluorobenzyl) {[(3′R,4S)-3′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexanecarboxylic acid;
N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}-N-(3-{[(trifluoromethyl)sulfonyl]amino}cyclobutyl)acetamide;
tert-butyl 3-[{[(1R)-5-bromo-2′,4′-dioxo-2,3-dihydro-3′H-spiro[indene-1,5′-[1,3]ozazolidin]-3′-yl]acetyl}(4-fluorobenzyl)amino]benzoate;
tert-butyl 3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]benzoate;
3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]benzoic acid;
2-[(4S)-5′-{1-[2-(dimethylamino)ethyl]-1H-pyrazol-4-yl}-2,5-dioxo-2′,3′-dihydro-1H-spiro[imidazolidine-4,1′-inden]-1-yl]-N-(4-fluorobenzyl)-N-[(2S)-1,1,1-trifluoropropan-2-yl]acetamide;
tert-butyl 4-({4-[(4S)-1-(2-{(4-fluorobenzyl)[(2S)-1,1,1-trifluoropropan-2-yl]amino}-2-oxoethyl)-2,5-dioxo-2′,3′-dihydrospiro[imidazolidine-4,1′-inden]-5′-yl]-1H-pyrazol-1-yl}methyl)piperidine-1-carboxylate;
2-{(4S)-2,5-dioxo-5′-[1-(piperidin-4-ylmethyl)-1H-pyrazol-4-yl]-2′,3′-dihydro-1H-spiro[imidazolidine-4,1′-inden]-1-yl}-N-(4-fluorobenzyl)-N-[(2S)-1,1,1-trifluoropropan-2-yl]acetamide;
N-(4-fluorobenzyl)-2-{6-[(1-methyl-1H-pyrazol-4-yl)amino]-1,1-dioxido-2′,5′-dioxo-1′H-spiro[1-benzothiophene-3,4′-imidazolidin]-1′-yl}-N-[(2S)-1,1,1-trifluoropropan-2-yl]acetamide;
N-(4-fluorobenzyl)-2-[(1S)-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(azetidin-3-yl)-N-(4-fluorobenzyl)-2-{(1R)-5-[(methylcarbamoyl)amino]-2′,4′-dioxo-2,3-dihydro-3′H-spiro[indene-1,5′-[1,3]ozazolidin]-3′-yl}acetamide;
ethyl ({3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]azetidin-1-yl}sulfonyl)carbamate;
2-[(1S)-5-bromo-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-(6-methoxy-1,1-dioxido-2′,5′-dioxo-1′H-spiro[1-benzothiophene-3,4′-imidazolidin]-1′-yl)-N-[(2S)-1,1,1-trifluoropropan-2-yl]acetamide;
N-(4-fluorobenzyl)-2-[(3 S)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide;
N-(4-fluorobenzyl)-2-[(3R)-6-(1-methyl-1H-pyrazol-4-yl)-1,1-dioxido-2′,5′-dioxo-1′H-spiro[1-benzothiophene-3,4′-imidazolidin]-1′-yl]-N-[(2S)-1,1,1-trifluoropropan-2-yl]acetamide;
N-(4-fluorobenzyl)-2-[(1 S,3R)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-benzyl-N-(1-cyclopropylethyl)-2-(2,5-dioxo-2′,3′-dihydro-1H-spiro[imidazolidine-4,1′-inden]-1-yl)acetamide;
N-(1-cyclopropylethyl)-2-(2,5-dioxo-2′,3′-dihydro-1H-spiro[imidazolidine-4,1′-inden]-1-yl)-N-(1H-imidazol-4-ylmethyl)acetamide;
N-[(1S)-3′-(2-{[(1S)-1-cyclopropylethyl](4-fluorobenzyl)amino}-2-oxoethyl)-2′,4′-dioxo-2,3-dihydrospiro[indene-1,5′-[1,3]ozazolidin]-5-yl]-D-valinamide;
N-benzyl-N-(1-cyclopropylethyl)-2-(2,2-dimethyl-2′,5′-dioxo-1′H-spiro[1-benzofuran-3,4′-imidazolidin]-1′-yl)acetamide;
N-benzyl-N-(1-cyclopropylethyl)-2-(1′,3-dimethyl-2,2′,5-trioxo-1′,2′-dihydro-1H-spiro[imidazolidine-4,3′-indol]-1-yl)acetamide;
N-benzyl-N-(1-cyclopropylethyl)-2-(2,5-dioxo-2′,3′-dihydro-1H-spiro[imidazolidine-4,1′-inden]-1-yl)-2-methylpropanamide;
N-benzyl-N-(1-cyclopropylethyl)-2-[2,5-dioxo-3-(prop-2-en-1-yl)-2′,3′-dihydro-1H-spiro[imidazolidine-4,1′-inden]-1-yl]acetamide;
N-(1-cyclopropylethyl)-2-(2,5-dioxo-2′,3′-dihydro-1H-spiro[imidazolidine-4,1′-inden]-1-yl)-N-(2,2,2-trifluoroethyl)acetamide;
N-(1-cyclopropylethyl)-2-(2,5-dioxo-2′,3′-dihydro-1H-spiro[imidazolidine-4,1′-inden]-1-yl)-N-[3-(trifluoromethoxy)benzyl]acetamide;
N-(but-2-yn-1-yl)-N-(1-cyclopropylethyl)-2-(2,5-dioxo-2′,3′-dihydro-1H-spiro[imidazolidine-4,1′-inden]-1-yl)acetamide;
N-(1-cyclopropylethyl)-N-{[6-(difluoromethoxy)naphthalen-2-yl]methyl}-2-(2,5-dioxo-2′,3′-dihydro-1H-spiro[imidazolidine-4,1′-inden]-1-yl)acetamide;
N-benzyl-N-(1-cyclopropylethyl)-2-[5′-(methylsulfonyl)-2,5-dioxo-2′,3′-dihydro-1H-spiro[imidazolidine-4,1′-inden]-1-yl]acetamide;
N-benzyl-N-(1-cyclobutylethyl)-2-(7′-methoxy-2,5-dioxo-2′,3′-dihydro-1H-spiro[imidazolidine-4,1′-inden]-1-yl)acetamide;
N-benzyl-N-(1-cyclopropylethyl)-2-(1-methyl-2′,5′-dioxo-5,6-dihydro-1H,1′H-spiro[cyclopenta[c]pyrazole-4,4′-imidazolidin]-1′-yl)acetamide;
N-benzyl-N-(1-cyclopropylethyl)-2-[5′-(hydroxymethyl)-3-methyl-2,5-dioxo-2′,3′-dihydro-1H-spiro[imidazolidine-4,1′-inden]-1-yl]acetamide;
N-[(1S)-1-cyclobutylethyl]-2-[(4R)-2,5-dioxo-2′,3′-dihydro-1H-spiro[imidazolidine-4,1′-inden]-1-yl]-N-(prop-2-yn-1-yl)acetamide;
N³-[(1S)-1-cyclobutylethyl]-N³-{[(4R)-2,5-dioxo-2′,3′-dihydro-1H-spiro[imidazolidine-4, 1′-inden]-1-yl]acetyl}-beta-alaninamide;
N-(biphenyl-4-ylmethyl)-N-[(1S)-1-cyclobutylethyl]-2-[(4R)-2,5-dioxo-2′,3′-dihydro-1H-spiro[imidazolidine-4,1′-inden]-1-yl]acetamide;
N²-[(1S)-1-cyclobutylethyl]-N²-{[(4R)-2,5-dioxo-2′,3′-dihydro-1H-spiro[imidazolidine-4, 1′-inden]-1-yl]acetyl}glycinamide;
N-[(1S)-1-cyclobutylethyl]-2-[(4R)-2,5-dioxo-2′,3′-dihydro-1H-spiro[imidazolidine-4,1′-inden]-1-yl]-N-[(1-methyl-2-oxo-1,2-dihydropyridin-4-yl)methyl]acetamide;
N-benzyl-N-[(1R)-1-cyclopropylethyl]-2-[(4R)-2,5-dioxo-2′,3′-dihydro-1H-spiro[imidazolidine-4,1′-inden]-1-yl]acetamide;
N-benzyl-N-[(1S)-1-cyclopropylethyl]-2-[(4S)-2,5-dioxo-2′,3′-dihydro-1H-spiro[imidazolidine-4,1′-inden]-1-yl]acetamide;
N-benzyl-N-[(1S)-1-cyclopropylethyl]-2-[(4R)-2,5-dioxo-2′,3′-dihydro-1H-spiro[imidazolidine-4,1′-inden]-1-yl]acetamide;
N-benzyl-N-[(1R)-1-cyclopropylethyl]-2-[(4S)-2,5-dioxo-2′,3′-dihydro-1H-spiro[imidazolidine-4,1′-inden]-1-yl]acetamide;
N-[4-(acetylamino)benzyl]-N-[(1S)-1-cyclobutylethyl]-2-[(4R)-2,5-dioxo-2′,3′-dihydro-1H-spiro[imidazolidine-4,1′-inden]-1-yl]acetamide;
N-[(1S)-1-cyclobutylethyl]-2-[(4R)-2,5-dioxo-2′,3′-dihydro-1H-spiro[imidazolidine-4,1′-inden]-1-yl]-N-(4-ethynylbenzyl)acetamide;
N-[(1S)-1-cyclobutylethyl]-2-[(4R)-2,5-dioxo-2′,3′-dihydro-1H-spiro[imidazolidine-4,1′-inden]-1-yl]-N-({1-[2-(methylamino)-2-oxoethyl]-1H-1,2,3-triazol-4-yl}methyl)acetamide;
2-{4-[([(1S)-1-cyclobutylethyl]{[(4R)-2,5-dioxo-2′,3′-dihydro-1H-spiro[imidazolidine-4,1′-inden]-1-yl]acetyl}amino)methyl]-1H-1,2,3-triazol-1-yl}-N,N-dimethylacetamide;
N³-[(1S)-1-cyclobutylethyl]-N³-{[(4R)-2,5-dioxo-2′,3′-dihydro-1H-spiro[imidazolidine-4, 1′-inden]-1-yl]acetyl}-N-methyl-beta-alaninamide;
N-benzyl-N-(1-cyclopropylethyl)-2-[5′-(6-hydroxypyridin-3-yl)-2,5-dioxo-2′,3′-dihydro-1H-spiro[imidazolidine-4,1′-inden]-1-yl]acetamide;
N-benzyl-N-[(1S)-1-cyclopropylethyl]-2-[5′-(formylamino)-2,5-dioxo-2′,3′-dihydro-1H-spiro[imidazolidine-4,1′-inden]-1-yl]acetamide;
N-benzyl-N-[(1S)-1-cyclopropylethyl]-2-[2′,4′-dioxo-5-(2H-tetrazol-5-yl)-2,3-dihydro-3′H-spiro[indene-1,5′-[1,3]ozazolidin]-3′-yl]acetamide;
2,2′-({[3′-(2-{benzyl[(1S)-1-cyclopropylethyl]amino}-2-oxoethyl)-2′,4′-dioxo-2,3-dihydrospiro[indene-1,5′-[1,3]ozazolidin]-5-yl]carbonyl}imino)diacetic acid;
N-benzyl-2-[(4R)-5′-bromo-2,5-dioxo-2′,3′-dihydro-1H-spiro[imidazolidine-4,1′-inden]-1-yl]-N-[(1S)-1-cyclopropylethyl]acetamide;
N-(4-fluorobenzyl)-2-[(1 S,3R)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-{(1 S,3R)-5-[1-(2-amino-2-oxoethyl)-1H-pyrazol-4-yl]-3-hydroxy-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(1S)-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[1,1-dioxido-2′,5′-dioxo-6-(pyridin-3-yl)-1′H-spiro[1-benzothiophene-3,4′-imidazolidin]-1′-yl]-N-(4-fluorobenzyl)-N-[(2S)-1,1,1-trifluoropropan-2-yl]acetamide;
2-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]benzoic acid;
methyl 3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanoate;
3-[(4-fluorobenzyl)({(1R)-5-[(methylcarbamoyl)amino]-2′,4′-dioxo-2,3-dihydro-3′H-spiro[indene-1,5′-[1,3]ozazolidin]-3′-yl}acetyl)amino]butanoic acid;
2-{(1S)-5-[1-(2-amino-2-oxoethyl)-1H-pyrazol-4-yl]-2′,4′-dioxo-2,3-dihydro-3′H-spiro[indene-1,5′-[1,3]ozazolidin]-3′-yl}-N-(4-fluorobenzyl)-N-[(2S)-1,1,1-trifluoropropan-2-yl]acetamide;
N-(4-fluorobenzyl)-2-[(3′S,4S)-3′-fluoro-5′-{1-[2-(methylamino)-2-oxoethyl]-1H-pyrazol-4-yl}-2,5-dioxo-2′,3′-dihydro-1H-spiro[imidazolidine-4,1′-inden]-1-yl]-N-[(2S)-1,1,1-trifluoropropan-2-yl]acetamide;
N-(4-fluorobenzyl)-2-[(1R,3R)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R,3R)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1 S,3S)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2-[(1S)-5-{1-[2-(dimethylamino)-2-oxoethyl]-1H-pyrazol-4-yl}-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1 S,3R)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyridin-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(pyridin-4-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(pyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(pyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-(6-oxo-1,6-dihydropyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-(6-oxo-1,6-dihydropyridin-3-yl)-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1R)-2′,4′-dioxo-5-phenyl-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
2-[(1S)-2′,4′-dioxo-5-phenyl-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-cyclohexyl-N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-(tetrahydro-2H-pyran-4-yl)acetamide;
3-[(4-fluorobenzyl){[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclobutanecarboxylic acid;
4-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexanecarboxylic acid;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-{trans-4-[(methylsulfonyl)amino]cyclohexyl}acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-(3-{[(trifluoromethyl)sulfonyl]amino}cyclobutyl)acetamide;
N-(4-fluorobenzyl)-2-[(1R)-5-(1-methyl-1H-pyrazol-4-yl)-2′,4′-dioxo-2,3-dihydro-3′H-spiro[indene-1,5′-[1,3]ozazolidin]-3′-yl]-N-[1-(methylsulfonyl)piperidin-4-yl]acetamide;
2-[(1R,3S)-5-bromo-3-hydroxy-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R,3S)-3-hydroxy-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
2,2′-({3-[(4-fluorobenzyl) {[(1R)-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclobutyl}imino)diacetic acid;
N-(4-fluorobenzyl)-2-[(1 S,3R)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R)-5-(1,2-oxazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1S)-5-(1,2-oxazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-[(1R,3S)-3-hydroxy-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
N-(4-fluorobenzyl)-2-{(1 S,3R)-3-fluoro-5-[(methylcarbamoyl)amino]-2′,4′-dioxo-2,3-dihydro-3′H-spiro[indene-1,5′-[1,3]ozazolidin]-3′-yl}-N-[(2S)-1,1,1-trifluoropropan-2-yl]acetamide;
(4-{[(5′-bromo-6′-fluoro-2,5-dioxo-2′,3′-dihydro-1H-spiro[imidazolidine-4,1′-inden]-1-yl)acetyl](4-fluorobenzyl)amino}cyclohexylidene)acetic acid;
{4-[(4-fluorobenzyl) {[6′-fluoro-5′-(1-methyl-1H-pyrazol-4-yl)-2,5-dioxo-2′,3′-dihydro-1H-spiro[imidazolidine-4,1′-inden]-1-yl]acetyl}amino]cyclohexylidene}acetic acid;
tert-butyl (4-{[(5′-bromo-6′-fluoro-2,5-dioxo-2′,3′-dihydro-1H-spiro[imidazolidine-4, 1′-inden]-1-yl)acetyl](4-fluorobenzyl)amino}cyclohexylidene)acetate;
2-[(1R,3S)-5-bromo-3-fluoro-2′,4′-dioxo-2,3-dihydro-3′H-spiro[indene-1,5′-[1,3]ozazolidin]-3′-yl]-N-(4-fluorobenzyl)-N-[(2S)-1,1,1-trifluoropropan-2-yl]acetamide;
N-(4-fluorobenzyl)-2-[(1R,3S)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide;
{4-[(4-fluorobenzyl) {[(1 S,3R)-3-fluoro-5-(1-methyl-1H-pyrazol-4-yl)-2′,4′-dioxo-2,3-dihydro-3′H-spiro[indene-1,5′-[1,3]ozazolidin]-3′-yl]acetyl}amino]cyclohexylidene}acetic acid;
N-(4-fluorobenzyl)-2-[(1R,3R)-3-fluoro-5-(1-methyl-1H-pyrazol-4-yl)-2′,4′-dioxo-2,3-dihydro-3′H-spiro[indene-1,5′-[1,3]ozazolidin]-3′-yl]-N-[(2S)-1,1,1-trifluoropropan-2-yl]acetamide; and
N-(4-fluorobenzyl)-2-[(1R,3R)-3-fluoro-5-{1-[2-(methylamino)-2-oxoethyl]-1H-pyrazol-4-yl}-2′,4′-dioxo-2,3-dihydro-3′H-spiro[indene-1,5′-[1,3]ozazolidin]-3′-yl]-N-[(2S)-1,1,1-trifluoropropan-2-yl]acetamide.

19. A pharmaceutical composition comprising a pharmaceutically acceptable exicipient and a therapeutically effective amount of a compound of claim 1 or a pharmaceutically acceptable salt thereof.

20. A method of treating cancer in a patient, comprising administering to a patient suffering from a cancer a therapeutically effective amount of a compound of claim 1, or a pharmaceutically acceptable salt thereof.

21. A method of treating metabolic disease, neurogenerative disorders or inflammation in a patient, comprising administering, to a patient suffering from metabolic disease, neurogenerative disorders or inflammation, a therapeutically effective amount of a compound of claim 1, or a pharmaceutically acceptable salt thereof.

22. The method of claim 20, wherein said cancer is selected from the group consisting of acoustic neuroma, acute leukemia, acute lymphoblastic leukemia, acute myelogenous leukemia, acute t-cell leukemia, basal cell carcinoma, bile duct carcinoma, bladder cancer, brain cancer, breast cancer, bronchogenic carcinoma, Burkitt's lymphoma, cervical cancer, chondrosarcoma, chordoma, choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, dysplasias, metaplasias, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, gastric carcinoma, germ cell testicular cancer, gestational trophobalstic disease, glioblastoma, head and neck cancer, heavy chain disease, hemangioblastoma, hepatoma, hepatocellular cancer, hormone insensitive prostate cancer, leiomyosarcoma, liposarcoma, lung cancer, lymphangioendothelio-sarcoma, lymphangiosarcoma, lymphoblastic leukemia, lymphoma, malignancies and hyperproliferative disorders of the bladder, breast, colon, lung, ovaries, pancreas, prostate, skin and uterus, lymphoid malignancies of T-cell or B-cell origin, leukemia, medullary carcinoma, medulloblastoma, melanoma, meningioma, mesothelioma, multiple myeloma, myelogenous leukemia, myeloma, myxosarcoma, neuroblastoma, oligodendroglioma, oral cancer, osteogenic sarcoma, ovarian cancer, pancreatic cancer, papillary adenocarcinomas, papillary carcinoma, peripheral T-cell lymphoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors, stomach cancer, squamous cell carcinoma, synovioma, sweat gland carcinoma, testicular cancer, thyroid cancer, Waldenström's macroglobulinemia, testicular tumors, uterine cancer, and Wilms' tumor.

23. The method of claim 20 or 21, further comprising administering a therapeutically effective amount of at least one additional therapeutic agent.
